(19) United States
(12) Patent Application Publication
(10) Pub. No.: US 2024/0415010 A1
(43) Pub. Date: Dec. 12, 2024

(54) COMPOUND, ORGANIC ELECTROLUMINESCENT ELEMENT MATERIAL, ORGANIC ELECTROLUMINESCENT ELEMENT, AND ELECTRONIC DEVICE

(71) Applicant: IDEMITSU KOSAN CO.,LTD., Tokyo (JP)

(72) Inventors: Shota TANAKA, Chiyoda-ku (JP); Tasuku HAKETA, Chiyoda-ku (JP); Yusuke TAKAHASHI, Chiyoda-ku (JP); Takuto FUKAMI, Chiyoda-ku (JP); Tsukasa SAWATO, Chiyoda-ku (JP)

(73) Assignee: IDEMITSU KOSAN CO.,LTD., Tokyo (JP)

(21) Appl. No.: 18/291,654

(22) PCT Filed: Aug. 26, 2022
(86) PCT No.: PCT/JP2022/032210

§ 371 Date: Jan. 24, 2024

(30) Foreign Application Priority Data
Aug. 27, 2021 (JP) 2021-139196

(51) Int. Cl. H10K 85/60; H10K 50/15

(52) U.S. Cl. CPC H10K 85/615; H10K 50/15; H10K 85/631; H10K 85/633; H10K 85/6572; H10K 85/6574

(57) ABSTRACT

A compound represented by the following formula (1): in which N*, R¹ to R⁹, L¹, L², L³, Ar¹ and Ar² are as defined in the disclosure. An organic electroluminescent device, including a cathode, an anode, and one or more organic layers intervening between the cathode and the anode, where the one or more organic layers includes a light emitting layer, and at least one layer of the one or more organic layers contains the compound. An electronic device including the organic electroluminescent device.

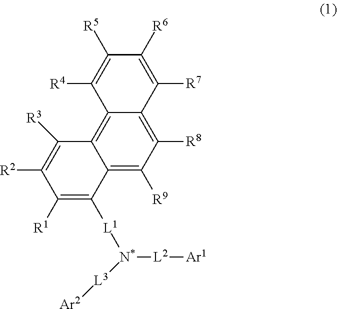

### TECHNICAL FIELD

The present invention relates to a compound, a material for organic electroluminescent devices, an organic electroluminescent device, and an electronic device including the organic electroluminescent device.

### BACKGROUND ART

In general, an organic electroluminescent device (which may be hereinafter referred to as an “organic EL device”) is constituted by an anode, a cathode, and an organic layer intervening between the anode and the cathode. In application of a voltage between both the electrodes, electrons from the cathode side and holes from the anode side are injected into a light emitting region, and the injected electrons and holes are recombined in the light emitting region to generate an excited state, which then returns to the ground state to emit light. Accordingly, development of a material that efficiently transports electrons or holes into the light emitting region, and promotes recombination of the electrons and holes is important for providing a high-performance organic EL device.

PTLs 1 to 4 describe compounds used for a material for organic electroluminescent devices.

### CITATION LIST

### Patent Literature

PTL 1: US 2017/0018710 A
PTL 2: KR 10-2020-0136116 A
PTL 3: KR 10-2020-0062616 A
PTL 4: KR 10-2020-0034584 A

### SUMMARY OF INVENTION

### Technical Problem

Various compounds for organic EL devices have been reported, but a compound that further enhances the capability of an organic EL device has been still demanded.

The present invention has been made to solve the above-mentioned problems, and an object of the present invention is to provide a compound that further improves the capability of an organic EL device, an organic EL device with further improved device capability, and an electronic device including such an organic EL device.

### Solution to Problem

As a result of intensive research on the capability of organic EL devices containing compounds described in PTLs 1 to 4, the present inventors have found that the capability of an organic EL device containing a compound represented by the following formula (1) is further improved.

In one embodiment, the present invention provides a compound represented by the following formula (1):

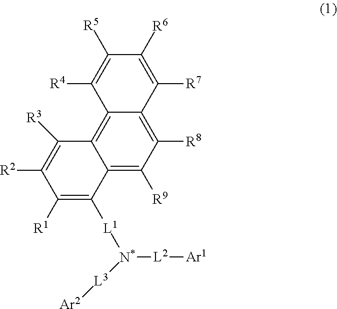

in the formula (1),
N* is a central nitrogen atom;
R¹ to R⁹ are each a hydrogen atom;
L¹ is an unsubstituted arylene group having 6 to 12 ring carbon atoms;
L² is a single bond or an unsubstituted arylene group having 6 to 12 ring carbon atoms;
L³ is a single bond or an unsubstituted arylene group having 6 to 12 ring carbon atoms;
Ar¹ is a group represented by any one of the following formulae (1-a) to (1-e); and
Ar² is a substituted or unsubstituted aryl group composed of only a 6-membered ring having 6 to 30 ring carbon atoms, or a group represented by the following formula (1-f) or (1-g):

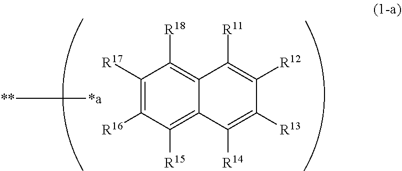

in the formula (1-a),
R¹¹ to R¹⁸ are each independently a hydrogen atom, a substituted or unsubstituted alkyl group having 1 to 6 carbon atoms, or a substituted or unsubstituted aryl group having 6 to 12 ring carbon atoms,
provided that,
one selected from R¹¹ to R¹⁸ is a single bond bonded to *a, and
adjacent two selected from R²¹ to R²⁸, which are not a single bond, are not bonded to each other, and thus do not form a ring structure; and
** represents a bonding site to L²,
provided that, when L² is a single bond, ** represents a bonding site to the central nitrogen atom N*,

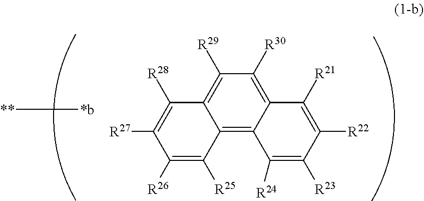

in the formula (1-b),
R²¹ to R³⁰ are each independently a hydrogen atom, a substituted or unsubstituted alkyl group having 1 to 6 carbon atoms, or a substituted or unsubstituted aryl group having 6 to 12 ring carbon atoms,
provided that,
one selected from R²¹ to R³⁰ is a single bond bonded to *b, and
adjacent two selected from R²¹ to R³⁰, which are not a single bond, are not bonded to each other, and thus do not form a ring structure; and
** represents a bonding site to L²,
provided that, when L² is a single bond, ** represents a bonding site to the central nitrogen atom N*,

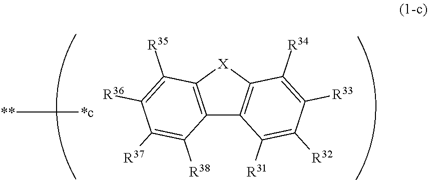

in the formula (1-c),
R³¹ to R³⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
X is an oxygen atom, a sulfur atom, or NR^{a};
R^{a} is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms,
provided that,
one selected from R³¹ to R³⁸ and R^{a} is a single bond bonded to *c, and
adjacent two selected from R³¹ to R³⁸ which are not a single bond may be bonded to each other to form a substituted or unsubstituted benzene ring; and
** represents a bonding site to L²,
provided that, when L² is a single bond, ** represents a bonding site to the central nitrogen atom N*,

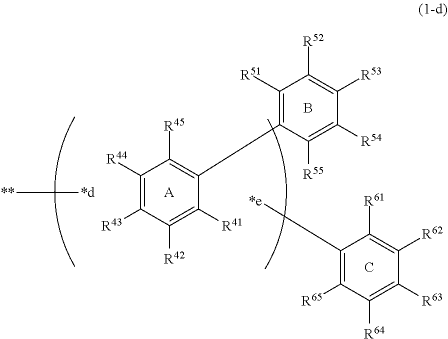

in the formula (1-d),
R⁴¹ to R⁴⁵ are each independently a hydrogen atom, an unsubstituted alkyl group having 1 to 6 carbon atoms, or an unsubstituted phenyl group;
R⁵¹ to R⁵⁵ and R⁶¹ to R⁶⁵ are each independently a hydrogen atom or an unsubstituted alkyl group having 1 to 6 carbon atoms,
provided that,
one selected from R⁴¹ to R⁴⁵ is a single bond bonded to *d,
another one selected from R⁴¹ to R⁴⁵ is a single bond bonded to *e,
adjacent two selected from R⁴¹ to R⁴⁵, which are not a single bond, are not bonded to each other, and thus do not form a ring structure, and
adjacent two selected from R⁵¹ to R⁵⁵ and R⁶¹ to R⁶⁵ may be bonded to each other to form a substituted or unsubstituted benzene ring; and
** represents a bonding site to L²,
provided that, when L² is a single bond, ** represents a bonding site to the central nitrogen atom N*,

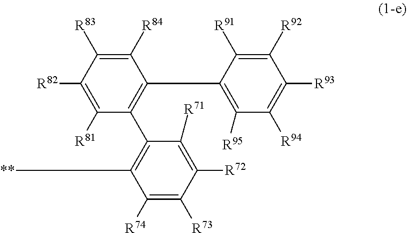

in the formula (1-e),
R⁷¹ to R⁷⁴, R⁸¹ to R⁸⁴, and R⁹¹ to R⁹⁵ are each independently a hydrogen atom, an unsubstituted alkyl group having 1 to 6 carbon atoms, or an unsubstituted phenyl group,
provided that,
adjacent two selected from R⁷¹ to R⁷⁴, R⁸¹ to R⁸⁴, and R⁹¹ to R⁹⁵ are not bonded to each other, and thus do not form a ring structure, and
** represents a bonding site to L²,
provided that, when L² is a single bond, ** represents a bonding site to the central nitrogen atom N*,

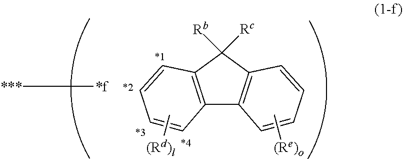

in the formula (1-f),
R^{b} and R^{c} are each independently an unsubstituted alkyl group having 1 to 6 carbon atoms, or an unsubstituted aryl group having 6 to 12 ring carbon atoms,
provided that,
when R^{b} and R^{c} are each an unsubstituted alkyl group having 1 to 6 carbon atoms, R^{b} and R^{c} are not bonded to each other and thus do not form a ring structure, and
when R^{b} and R^{c} are each an unsubstituted aryl group having 6 to 12 ring carbon atoms, R^{b} and R^{c} may be bonded to each other to form a substituted or unsubstituted ring;
R^{d} and R^{e} are each independently an unsubstituted aryl group having 6 to 12 ring carbon atoms;
l and o are each independently an integer of 0 to 2, in a case where 1 is 0, R^{d} is not present, and in a case where o is 0, R^{e} is not present;
*f is bonded to one carbon atom selected from carbon atoms *1 to *4 and a carbon atom contained in an unsubstituted alkyl group having 1 to 6 carbon atoms or an unsubstituted aryl group having 6 to 12 ring carbon atoms represented by R^{b} and R^{c}; and
*** represents a bonding site to L³,
provided that, when L³ is a single bond, ** represents a bonding site to the central nitrogen atom N*,

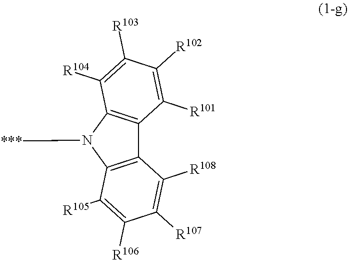

in the formula (1),
R¹⁰¹ to R¹⁰⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms,
provided that,
adjacent two selected from R¹⁰¹ to R¹⁰⁸ may be bonded to each other to form a substituted or unsubstituted benzene ring; and
*** represents a bonding site to L³,
provided that, when L³ is a single bond, *** represents a bonding site to the central nitrogen atom N*.

In another embodiment, the present invention provides a material for an organic EL device containing the compound represented by the formula (1).

In still another embodiment, the present invention provides an organic electroluminescent device including a cathode, an anode, and one or more organic layers intervening between the cathode and the anode, the organic layers including a light emitting layer, at least one layer of the organic layers containing the compound represented by the formula (1).

In a further embodiment, the present invention provides an electronic device including the organic electroluminescent device.

### Advantageous Effects of Invention

An organic EL device containing the compound represented by the formula (1) shows an improved device capability.

### BRIEF DESCRIPTION OF DRAWINGS

FIG. 1 is a schematic view showing an example of a layer configuration of an organic EL device according to an embodiment of the present invention.

FIG. 2 is a schematic view showing another example of a layer configuration of an organic EL device according to an embodiment of the present invention.

FIG. 3 is a schematic view showing still another example of a layer configuration of an organic EL device according to an embodiment of the present invention.

### DESCRIPTION OF EMBODIMENTS

### Definitions

In the description herein, the hydrogen atom encompasses isotopes thereof having different numbers of neutrons, i.e., protium, deuterium, and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocyclic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenylfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly, a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly, a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly, a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly, a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atom in the ZZ group is substituted by a substituent. The expression “substituted” in the expression “BB group substituted by an AA group” similarly means that one or more hydrogen atom in the BB group is substituted by the AA group.

### Substituents in Description

The substituents described in the description herein will be explained.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms of the “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

### Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atom of the “unsubstituted aryl group” by a substituent. Examples of the “substituted aryl group” include groups formed by one or more hydrogen atom of each of the “unsubstituted aryl groups” in the set of specific examples G1A by a substituent, and the examples of the substituted aryl groups in the set of specific examples G1B. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

### Unsubstituted Aryl Group (Set of Specific Examples G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

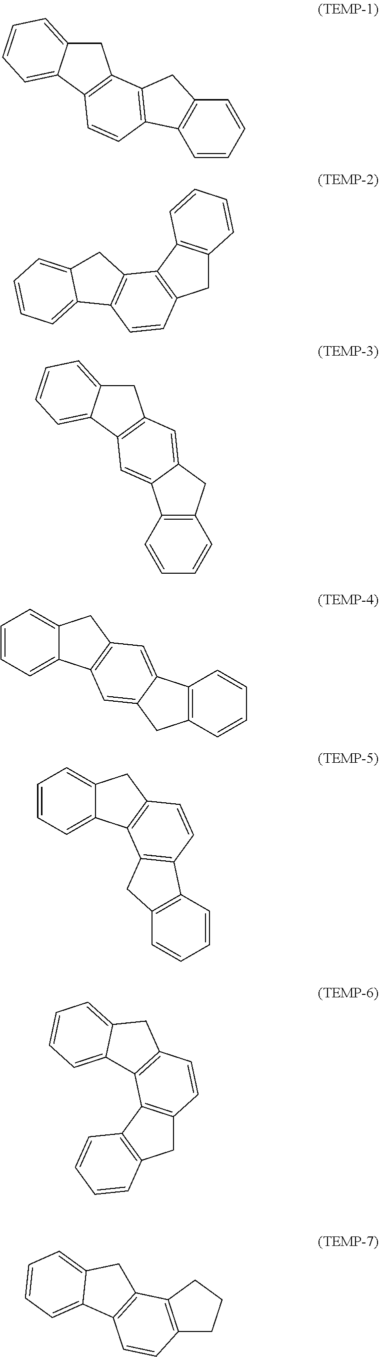

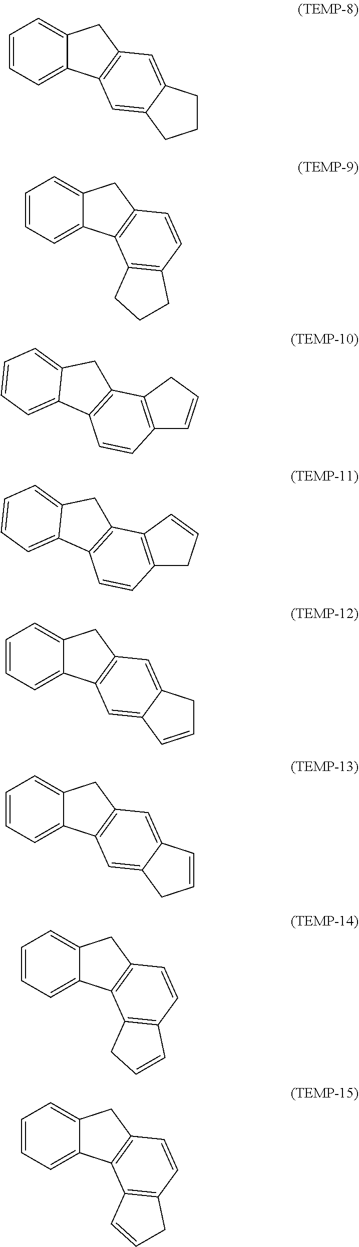

### Substituted Aryl Group (Set of Specific Examples G1B):

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
a o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
groups formed by substituting one or more hydrogen atom of each of monovalent aryl groups derived from the ring structures represented by the general formulae (TEMP-1) to (TEMP-15) by a substituent.

### Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom.

In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below. (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atom of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atom of each of monovalent heterocyclic groups derived from the ring structures represented by the following general formulae (TEMP-16) to (TEMP-33) by a substituent (set of specific examples G2B4).

### Unsubstituted Heterocyclic Group Containing Nitrogen Atom (Set of Specific Examples G2A1):

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.
   Unsubstituted Heterocyclic Group containing Oxygen Atom (Set of Specific Examples G2A2):
a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.
   Unsubstituted Heterocyclic Group containing Sulfur Atom (Set of Specific Examples G2A3):
a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).
   Monovalent Heterocyclic Group derived by removing One Hydrogen Atom from Ring Structures represented by General Formulae (TEMP-16) to (TEMP-33) (Set of Specific Examples G2A4)

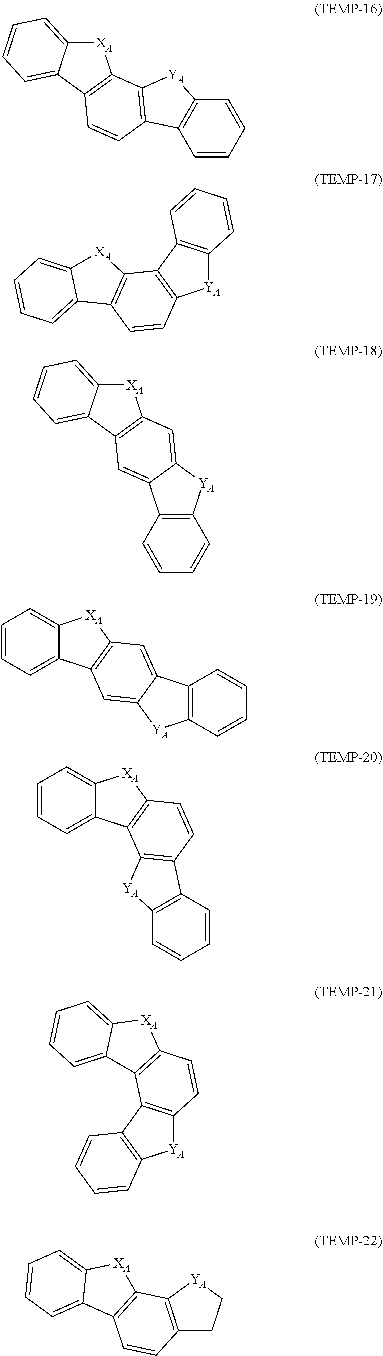

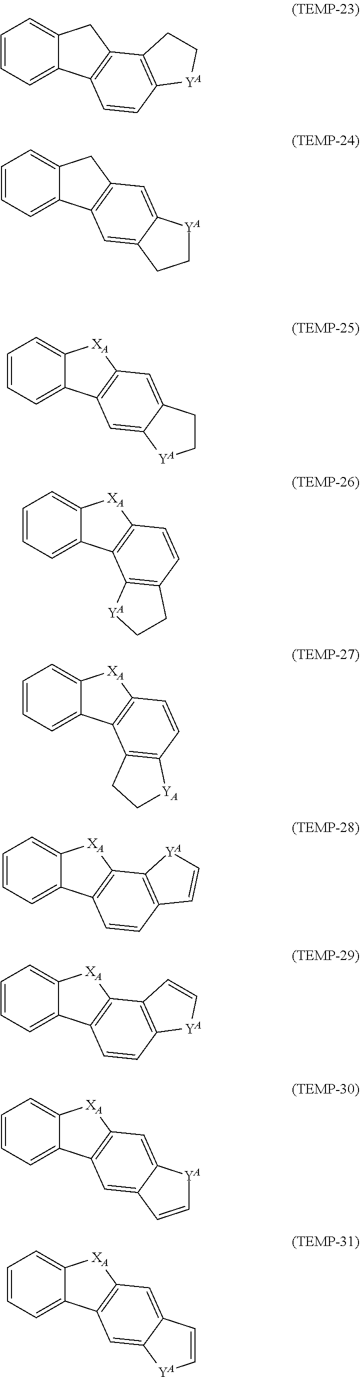

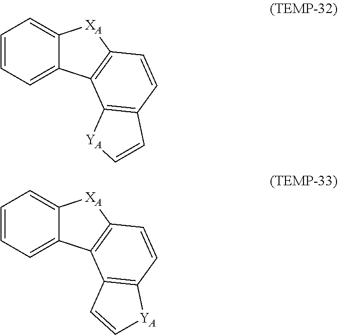

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

Substituted Heterocyclic Group containing Nitrogen Atom (Set of Specific Examples G2B1):
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and
a biphenylquinazolinyl group.
   Substituted Heterocyclic Group containing Oxygen Atom (Set of Specific Examples G2B2):
a phenyldibenzofuranyl group,
a methyldibenzofuranyl group,
a t-butyldibenzofuranyl group, and
a monovalent residual group of spiro[9H-xanthene-9,9′-[9H]fluorene].
   Substituted Heterocyclic Group containing Sulfur Atom (Set of Specific Examples G2B3):
a phenyldibenzothiophenyl group,
a methyldibenzothiophenyl group,
a t-butyldibenzothiophenyl group, and
a monovalent residual group of spiro[9H-thioxanthene-9,9′-[9H]fluorene].

Group formed by substituting one or more Hydrogen Atom of Monovalent Heterocyclic Group derived from Ring Structures represented by General Formulae (TEMP-16) to (TEMP-33) by Substituent (Set of Specific Examples G2B4)

The “one or more hydrogen atom of the monovalent heterocyclic group” means one or more hydrogen atom selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A} represents CH₂.

### Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atom of the “unsubstituted alkyl group” by a substituent. Specific examples of the “substituted alkyl group” include groups formed by substituting one or more hydrogen atom of each of the “unsubstituted alkyl groups” (set of specific examples G3A) by a substituent, and the examples of the substituted alkyl groups (set of specific examples G3B). In the description herein, the alkyl group in the “unsubstituted alkyl group” means a chain-like alkyl group. Accordingly, the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

### Unsubstituted Alkyl Group (Set of Specific Examples G3A):

a methyl group,
an ethyl group,
a n-propyl group,
an isopropyl group,
a n-butyl group,
an isobutyl group,
a s-butyl group, and
a t-butyl group.

### Substituted Alkyl Group (Set of Specific Examples G3B):

a heptafluoropropyl group (including isomers),
a pentafluoroethyl group,
a 2,2,2-trifluoroethyl group, and
a trifluoromethyl group.

### Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and the substituted alkenyl group means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atom of the “unsubstituted alkenyl group” by a substituent. Specific examples of the “substituted alkenyl group” include the “unsubstituted alkenyl groups” (set of specific examples G4A) that each have a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

### Unsubstituted Alkenyl Group (Set of Specific Examples G4A):

a vinyl group,
an allyl group,
a 1-butenyl group,
a 2-butenyl group, and
a 3-butenyl group.

### Substituted Alkenyl Group (Set of Specific Examples G4B):

a 1,3-butanedienyl group,
a 1-methylvinyl group,
a 1-methylallyl group,
a 1,1-dimethylallyl group,
a 2-methylallyl group, and
a 1,2-dimethylallyl group.

### Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”.

The “substituted alkynyl group” means a group formed by substituting one or more hydrogen atom of the “unsubstituted alkynyl group” by a substituent. Specific examples of the “substituted alkenyl group” include groups formed by substituting one or more hydrogen atom of the “unsubstituted alkynyl group” (set of specific examples G5A) by a substituent.

### Unsubstituted Alkynyl Group (Set of Specific Examples G5A):

an ethynyl group.

### Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below. (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atom of the “unsubstituted cycloalkyl group” by a substituent. Specific examples of the “substituted cycloalkyl group” include groups formed by substituting one or more hydrogen atom of each of the “unsubstituted cycloalkyl groups” (set of specific examples G6A) by a substituent, and the example of the substituted cycloalkyl group (set of specific examples G6B). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atom bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

### Unsubstituted Cycloalkyl Group (Set of Specific Examples G6A):

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

### Substituted Cycloalkyl Group (Set of Specific Examples G6B):

a 4-methylcyclohexyl group.
   Group represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —Si(G1)(G1)(G1) are the same as or different from each other.

Plural groups represented by G2 in —Si(G1)(G2)(G2) are the same as or different from each other.

Plural groups represented by G1 in —Si(G1)(G1)(G2) are the same as or different from each other.

Plural groups represented by G2 in —Si(G2)(G2)(G2) are the same as or different from each other.

Plural groups represented by G3 in —Si(G3)(G3)(G3) are the same as or different from each other.

Plural groups represented by G6 in —Si(G6)(G6)(G6) are the same as or different from each other.

### Group Represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

### Group Represented by —S—(R₉₀₅)

In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group Represented by —N(R₉₀₆)(R₉₀₇)

In the description herein, specific examples (set of specific examples G10) of the group represented by —N(R₉₀₆)(R₉₀₇) include:
—N(G1)(G1),
—N(G2)(G2),
—N(G1)(G2),
—N(G3)(G3), and
—N(G6)(G6).
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

### Halogen Atom

In the description herein, specific examples (set of specific examples G11) of the “halogen atom” include a fluorine atom, a chlorine atom, a bromine atom, and an iodine atom.

### Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atom of the “fluoroalkyl group” by a substituent. In the description herein, the “substituted fluoroalkyl group” encompasses a group formed by substituting one or more hydrogen atom bonded to the carbon atom of the alkyl chain in the “substituted fluoroalkyl group” by a substituent, and a group formed by substituting one or more hydrogen atom of the substituent in the “substituted fluoroalkyl group” by a substituent. Specific examples of the “unsubstituted fluoroalkyl group” include examples of groups formed by substituting one or more hydrogen atom in each of the “alkyl group” (set of specific examples G3) by a fluorine atom.

### Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atom of the “haloalkyl group” by a substituent. In the description herein, the “substituted haloalkyl group” encompasses a group formed by substituting one or more hydrogen atom bonded to the carbon atom of the alkyl chain in the “substituted haloalkyl group” by a substituent, and a group formed by substituting one or more hydrogen atom of the substituent in the “substituted haloalkyl group” by a substituent. Specific examples of the “unsubstituted haloalkyl group” include examples of groups formed by substituting one or more hydrogen atom in each of the “alkyl group” (set of specific examples G3) by a halogen atom. A haloalkyl group may be referred to as a halogenated alkyl group in some cases.

### Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

### Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

### Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

### Substituted or Unsubstituted Arylthio Group

In the description herein, specific examples of the “substituted or unsubstituted arylthio group” include a group represented by —S(G1), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

### Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “substituted or unsubstituted trialkylsilyl group” is 1 to 50, preferably 1 to 20, and more preferably 1 to 6, unless otherwise indicated in the description.

### Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly, the “aralkyl group” is a group formed by substituting a hydrogen atom of an “alkyl group” by an “aryl group” as a substituent, and is one embodiment of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

In the description herein, the substituted or unsubstituted aryl group is preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl, group, a 2-carbazolyl, group, a 3-carbazolyl, group, a 4-carbazolyl, group, or a 9-carbazolyl, group), a benzocarbazolyl group, an azacarbazolyl group, a diazacarbazolyl group, a dibenzofuranyl group, a naphthobenzofuranly group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (e.g., a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

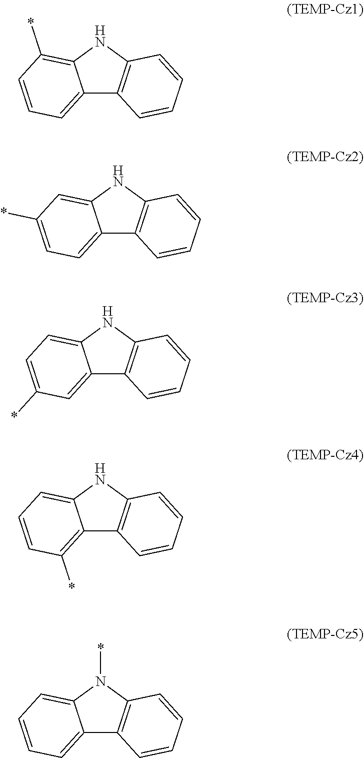 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In the description herein, the (9-phenyl)carbazolyl group is specifically any one of the following groups unless otherwise indicated in the description.

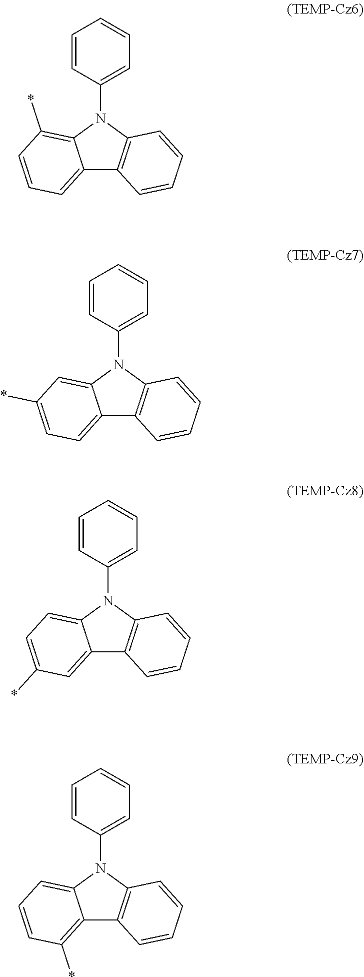 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulae (TEMP-Cz1) to (TEMP-Cz9), * represents a bonding site.

In the description herein, the dibenzofuranyl group and the dibenzothiophenyl group are specifically any one of the following groups unless otherwise indicated in the description.

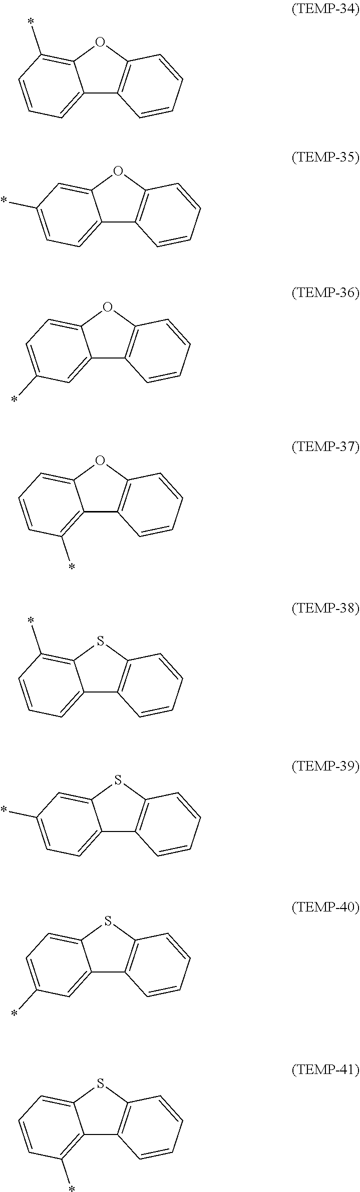

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

### Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

### Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

### Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl chain from the “substituted or unsubstituted alkyl groups” described in the set of specific examples G3.

In the description herein, the substituted or unsubstituted arylene group is preferably any one of the groups represented by the following general formulae (TEMP-42) to (TEMP-68) unless otherwise indicated in the description.

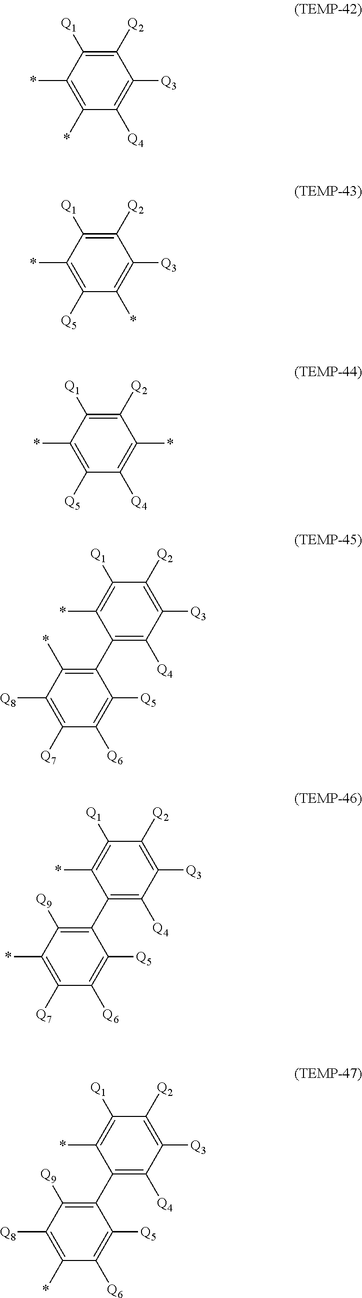

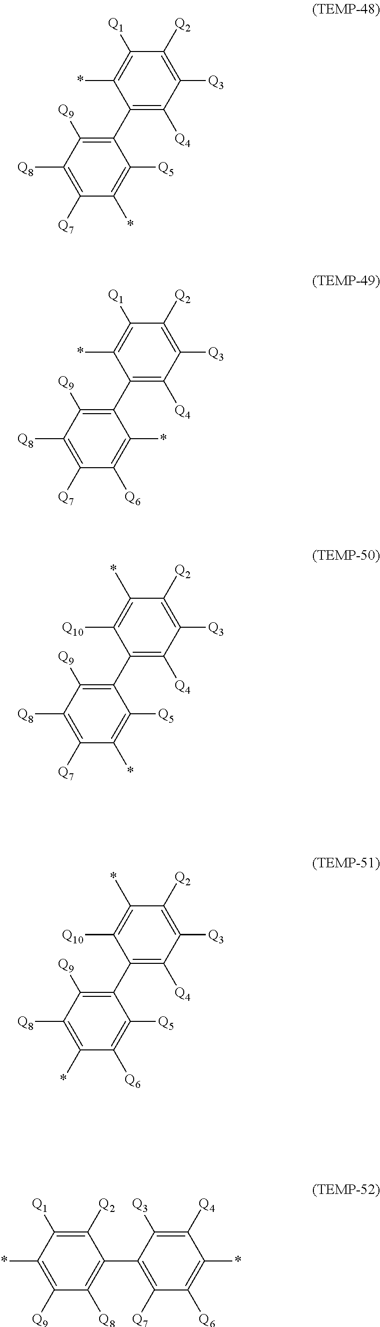

In the general formulae (TEMP-42) to (TEMP-52), Q₁ to Q₁₀ each independently represent a hydrogen atom or a substituent.

In the general formulae (TEMP-42) to (TEMP-52), * represents a bonding site.

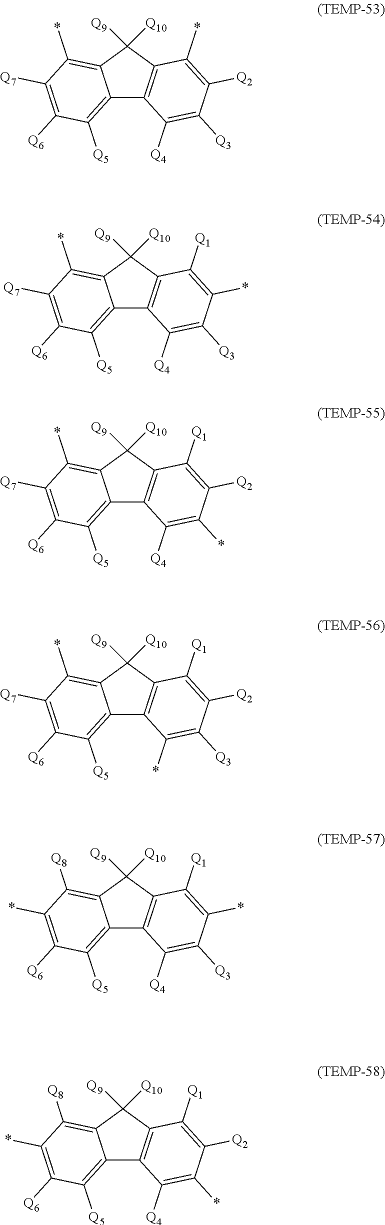

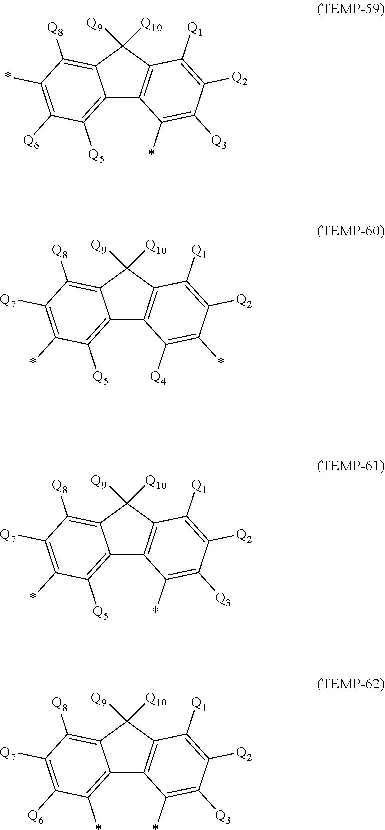

In the general formulae (TEMP-53) to (TEMP-62), Q₁ to Q₁₀ each independently represent a hydrogen atom or a substituent.

The formulae Q₉ and Q₁₀ may be bonded to each other to form a ring via a single bond.

In the general formulae (TEMP-53) to (TEMP-62), * represents a bonding site.

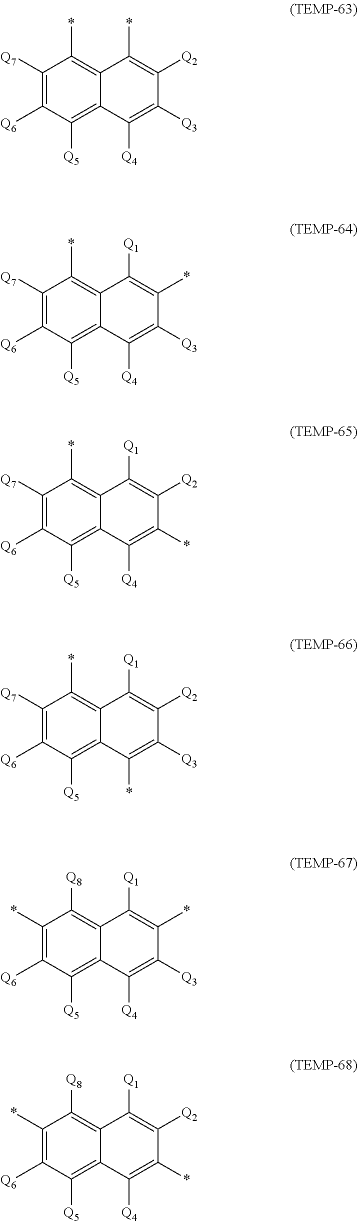

In the general formulae (TEMP-63) to (TEMP-68), Q₁ to Q₈ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

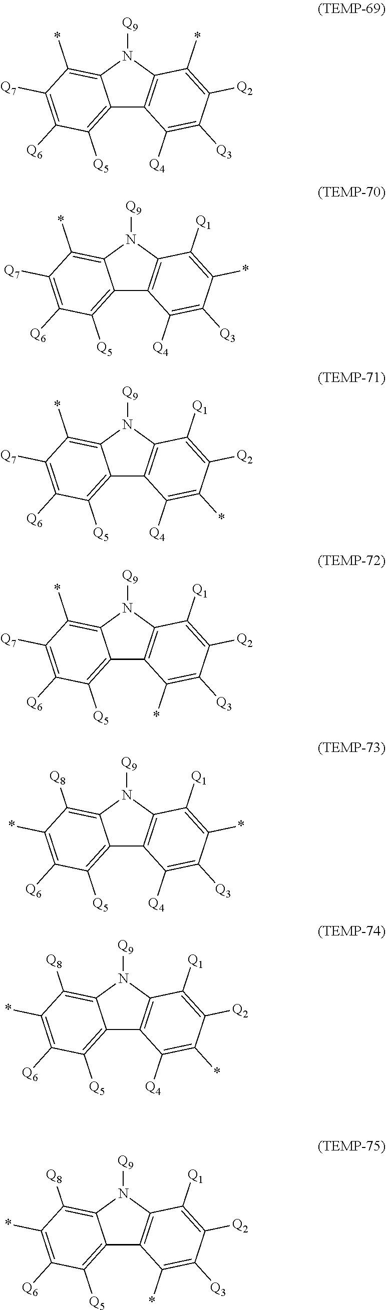

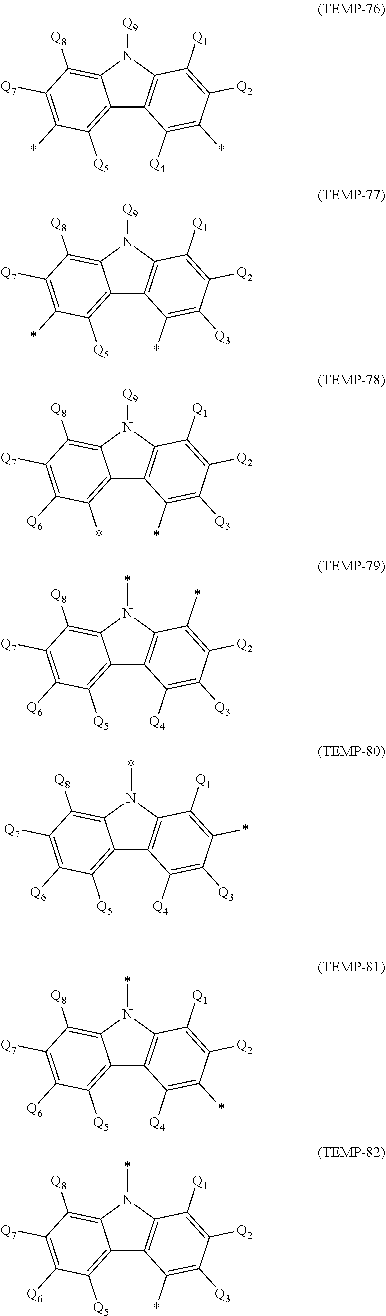

In the general formulae (TEMP-69) to (TEMP-82), Q₁ to Q₉ each independently represent a hydrogen atom or a substituent.

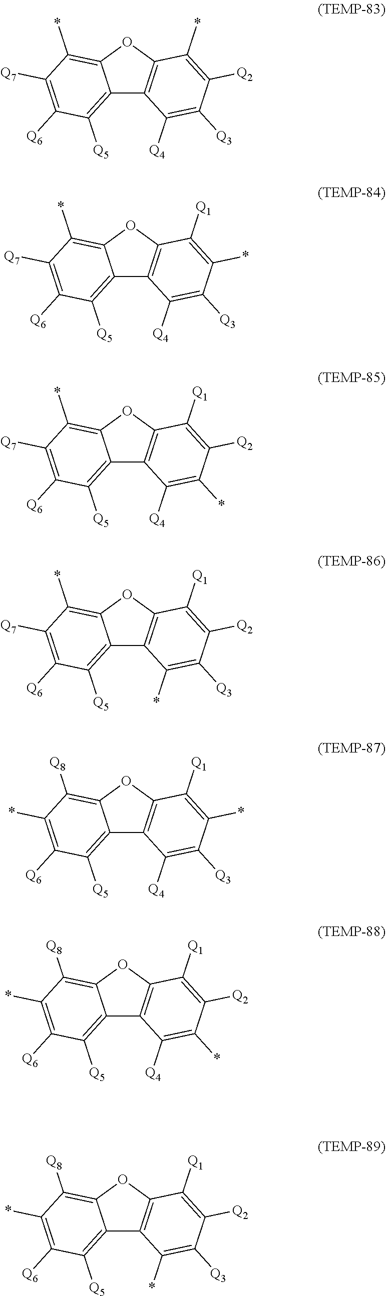

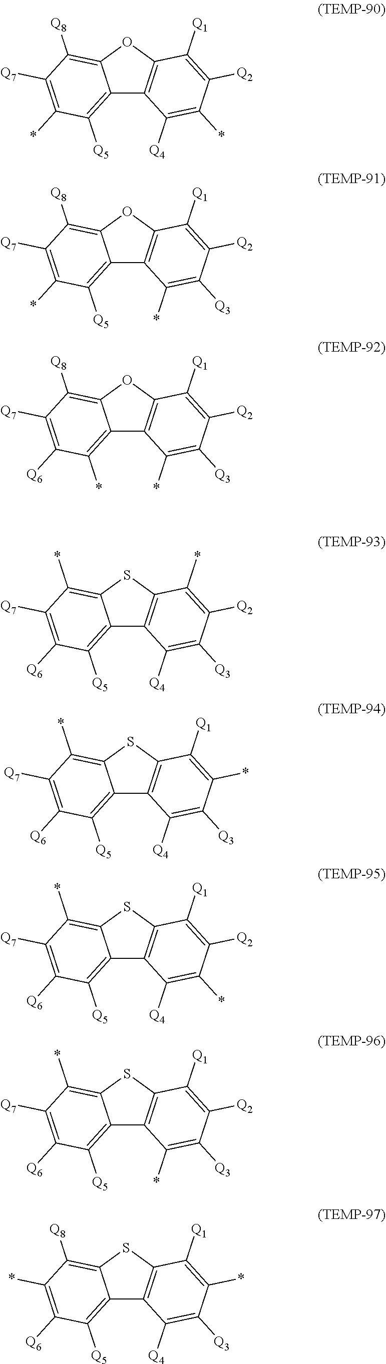

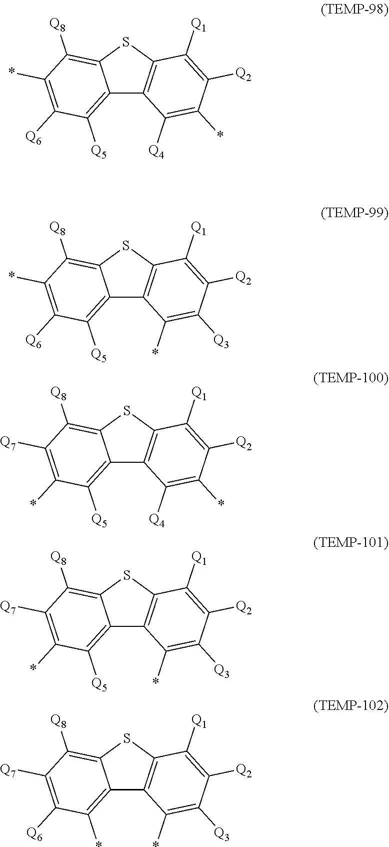

In the general formulae (TEMP-83) to (TEMP-102), Q₁ to Q₈ each independently represent a hydrogen atom or a substituent.

The above are the explanation of the “substituents in the description herein”.

### Case Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which may be hereinafter collectively referred to as a “case forming a ring by bonding”) will be explained below. The cases will be explained for the anthracene compound represented by the following general formula (TEMP-103) having an anthracene core skeleton as an example.

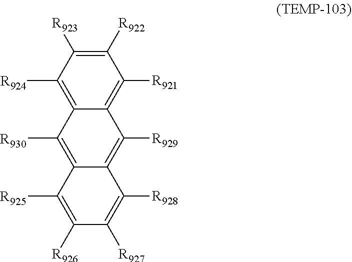

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂₉ and R₉₂₁.

The “one or more combinations” mean that two or more combinations each including adjacent two or more may form rings simultaneously. For example, in the case where R₉₂₁ and R₉₂₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

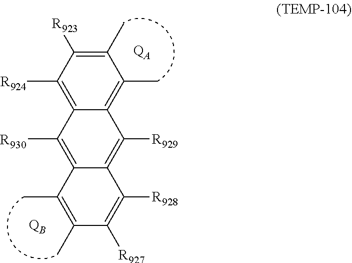

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂₂ and R₉₂₃ are bonded to each other to form a ring Q_{C}, and adjacent three (R₉₂₁, R₉₂₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Q_{C} share R₉₂₂.

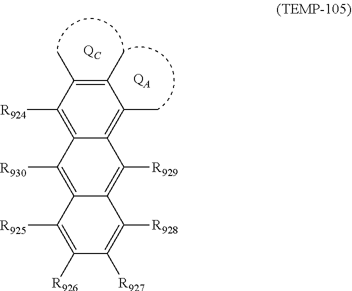

The formed “monocyclic ring” or “condensed ring” may be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Q_{C} formed in the general formula (TEMP-105) each are a “condensed ring”. The ring Q_{A} and the ring Q_{C} in the general formula (TEMP-105) form a condensed ring through condensation of the ring Q_{A} and the ring Q_{C}. In the case where the ring Q_{A} in the general formula (TMEP-104) is a benzene ring, the ring Q_{A} is a monocyclic ring. In the case where the ring Q_{A} in the general formula (TMEP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or anon-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring may be terminated with a hydrogen atom or the like, and may be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of an element selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more” each are “bonded to each other to form a substituted or unsubstituted monocyclic ring”, and the case where “one or more combinations of combinations each including adjacent two or more” each are “bonded to each other to form a substituted or unsubstituted condensed ring” (i.e., the “case forming a ring by bonding”).

### Substituent for “Substituted or Unsubstituted”

In one embodiment in the description herein, the substituent for the case of “substituted or unsubstituted” (which may be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms
a substituted or unsubstituted cycloalkyl group having 3 to 50 ring carbon atoms,
a substituted or unsubstituted aryl group having 6 to 50 ring carbon atoms, or
a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R₉₀₂ are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by R₉₀₅ exist, the two or more groups each represented by R₉₀₅ are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” may be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” may be a group selected from the group consisting of
an alkyl group having 1 to 18 carbon atoms,
an aryl group having 6 to 18 ring carbon atoms, and
a heterocyclic group having 5 to 18 ring atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has may be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

The compound of the present invention will be described below.

The compound of the present invention is represented by the following formula (1). However, hereinafter, the compound of the present invention represented by the formula (1) and formulae included in the formula (1) described later may be simply referred to as “inventive compound”.

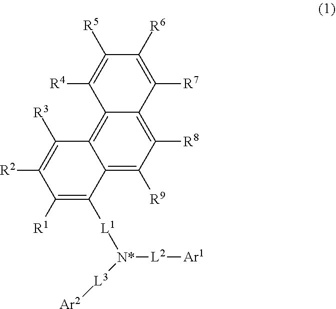

Hereinafter, symbols in the formula (1) and formulae included in the formula (1) described later will be described. The same symbols have the same meanings.

In the formula (1),
N* is a central nitrogen atom;
R¹ to R⁹ are each a hydrogen atom;
L¹ is an unsubstituted arylene group having 6 to 12 ring carbon atoms;
L² is a single bond or an unsubstituted arylene group having 6 to 12 ring carbon atoms; and
L³ is a single bond or an unsubstituted arylene group having 6 to 12 ring carbon atoms.

The unsubstituted arylene group having 6 to 12 ring carbon atoms represented by L¹ is preferably a phenylene group or a biphenylene group, and more preferably a phenylene group.

The inventive compound is preferably a compound represented by the following formula (1A) or (1B), and more preferably a compound represented by the following formula (1A).

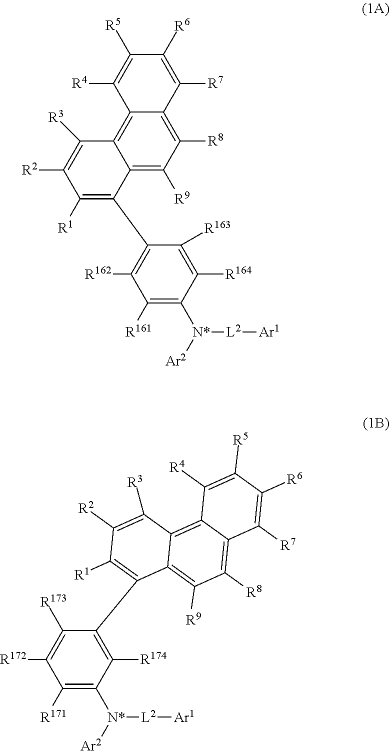

In the formulae (1A) and (1B), R¹⁶¹ to R¹⁶⁴ and R¹⁷¹ to R¹⁷⁴ are each a hydrogen atom; and N*, R¹ to R⁹, L², Ar¹, and Ar² are as defined in the formula (1).

The unsubstituted arylene group having 6 to 12 ring carbon atoms represented by L² is preferably a phenylene group or a biphenylene group, more preferably a phenylene group, still more preferably an o-phenylene group or a p-phenylene group, and even more preferably a p-phenylene group.

L² is preferably an unsubstituted arylene group having 6 to 12 ring carbon atoms, more preferably a phenylene group, still more preferably an o-phenylene group or a p-phenylene group, and even more preferably a p-phenylene group.

The unsubstituted arylene group having 6 to 12 ring carbon atoms represented by L³ is preferably a phenylene group or a biphenylene group, more preferably a phenylene group, still more preferably an o-phenylene group or a p-phenylene group, and even more preferably a p-phenylene group.
L³ is preferably an unsubstituted arylene group having 6 to 12 ring carbon atoms, more preferably a phenylene group, still more preferably an o-phenylene group or a p-phenylene group, and even more preferably a p-phenylene group.
Ar¹ is a group represented by any one of the following formulae (1-a) to (1-e).
Ar² is a substituted or unsubstituted aryl group composed of only a 6-membered ring having 6 to 30 ring carbon atoms, or a group represented by the following formula (1-f) or (1-g).

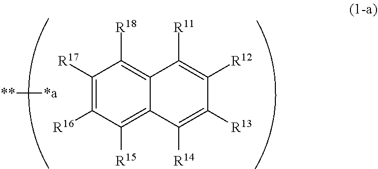

In the formula (1-a),
R¹¹ to R¹⁸ are each independently a hydrogen atom, a substituted or unsubstituted alkyl group having 1 to 6 carbon atoms, or a substituted or unsubstituted aryl group having 6 to 12 ring carbon atoms,
provided that,
one selected from R¹¹ to R¹⁸ is a single bond bonded to *a, and
adjacent two selected from R¹¹ to R¹⁸, which are not a single bond, are not bonded to each other, and thus do not form a ring structure; and
** represents a bonding site to L²,
provided that, when L² is a single bond, ** represents a bonding site to the central nitrogen atom N*.

The substituted or unsubstituted alkyl group having 1 to 6 carbon atoms represented by R¹¹ to R¹⁸ is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

The substituted or unsubstituted aryl group having 6 to 12 ring carbon atoms represented by R¹¹ to R¹⁸ is preferably a phenyl group, a biphenyl group, or a naphthyl group, more preferably a phenyl group or a naphthyl group, and still more preferably a phenyl group.

Each of R¹¹ to R¹⁸, which is not a single bond, is preferably a hydrogen atom.

In one embodiment of the present invention, one selected from R¹¹, R¹⁴, R¹⁵, and R¹⁸ is preferably a single bond bonded to *a.

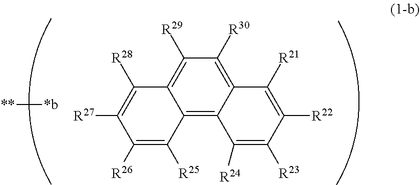

In the formula (1-b),
R²¹ to R³⁰ are each independently a hydrogen atom, a substituted or unsubstituted alkyl group having 1 to 6 carbon atoms, or a substituted or unsubstituted aryl group having 6 to 12 ring carbon atoms,
provided that,
one selected from R²¹ to R³⁰ is a single bond bonded to *b, and
adjacent two selected from R²¹ to R³⁰, which are not a single bond, are not bonded to each other, and thus do not form a ring structure; and
** represents a bonding site to L²,
provided that, when L² is a single bond, ** represents a bonding site to the central nitrogen atom N*.

The substituted or unsubstituted alkyl group having 1 to 6 carbon atoms represented by R²¹ to R³⁰ is as described above for R¹¹ to R¹⁸, and preferred groups and the like are also the same.

The substituted or unsubstituted aryl group having 6 to 12 ring carbon atoms represented by R²¹ to R³⁰ is as described above for R¹¹ to R¹⁸, and preferred groups and the like are also the same.

Each of R²¹ to R³⁰, which is not a single bond, is preferably a hydrogen atom.

In one embodiment of the present invention, one selected from R²¹, and R²⁸ to R³⁰ is preferably a single bond bonded to *b.

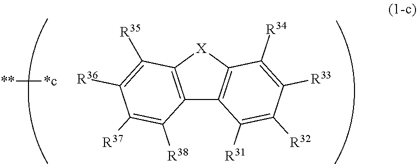

In the formula (1-c),
R³¹ to R³⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
X is an oxygen atom, a sulfur atom, or NR^{a};
R^{a} is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms,
provided that,
one selected from R³¹ to R³⁸ and R^{a} is a single bond bonded to *c, and
adjacent two selected from R³¹ to R³⁸ which are not a single bond may be bonded to each other to form a substituted or unsubstituted benzene ring; and
** represents a bonding site to L²,
provided that, when L² is a single bond, ** represents a bonding site to the central nitrogen atom N*.

The substituted or unsubstituted alkyl group having 1 to 6 carbon atoms represented by R³¹ to R³⁸ is as described above for R¹¹ to R¹⁸, and preferred groups and the like are also the same.

The substituted or unsubstituted aryl group having 6 to 12 ring carbon atoms represented by R³¹ to R³⁸ is as described above for R¹¹ to R¹⁸, and preferred groups and the like are also the same.

The substituted or unsubstituted aromatic heterocyclic group having 5 to 13 ring atoms represented by R³¹ to R³⁸ is preferably a dibenzothiophenyl group, a dibenzofuranyl group, or a carbazolyl group.

Each of R³¹ to R³⁸, which is not a single bond, is preferably a hydrogen atom.
X is preferably an oxygen atom or NR^{a}, and more preferably an oxygen atom.

The substituted or unsubstituted alkyl group having 1 to 6 carbon atoms represented by R^{a} is preferably a methyl group.

The substituted or unsubstituted aryl group having 6 to 12 ring carbon atoms represented by R^{a} is preferably a phenyl group or a naphthyl group, and more preferably a phenyl group.

R^{a} which is not a single bond is preferably a substituted or unsubstituted aryl group having 6 to 12 ring carbon atoms, and more preferably a phenyl group.

In one embodiment of the present invention, preferably one selected from R³¹, R³⁴, R³⁵, R³⁸, and R^{a}, more preferably one selected from R³⁴ and R³⁵, is a single bond bonded to *c.

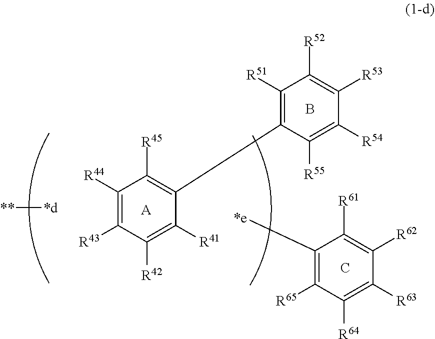

In the formula (1-d),
R⁴¹ to R⁴⁵ are each independently a hydrogen atom, an unsubstituted alkyl group having 1 to 6 carbon atoms, or an unsubstituted phenyl group;
R⁵¹ to R⁵⁵ and R⁶¹ to R⁶⁵ are each independently a hydrogen atom or an unsubstituted alkyl group having 1 to 6 carbon atoms,
provided that,
one selected from R⁴¹ to R⁴⁵ is a single bond bonded to *d,
another one selected from R⁴¹ to R⁴⁵ is a single bond bonded to *e,
adjacent two selected from R⁴¹ to R⁴⁵, which are not a single bond, are not bonded to each other, and thus do not form a ring structure, and
adjacent two selected from R⁵¹ to R⁵⁵ and R⁶¹ to R⁶⁵ may be bonded to each other to form a substituted or unsubstituted benzene ring; and
** represents a bonding site to L²,
provided that, when L² is a single bond, ** represents a bonding site to the central nitrogen atom N*.

The unsubstituted alkyl group having 1 to 6 carbon atoms represented by R⁴¹ to R⁴⁵ is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, and still more preferably a methyl group or a t-butyl group.

Each of R⁴¹ to R⁴⁵, which is not a single bond, is preferably a hydrogen atom.

The unsubstituted alkyl groups having 1 to 6 carbon atoms represented by R⁵¹ to R⁵⁵ and R⁶¹ to R⁶⁵ are as described above for R⁴¹ to R⁴⁵, and preferred groups and the like are also the same.

Each of R⁵¹ to R⁵⁵, which is not a single bond, is preferably a hydrogen atom.

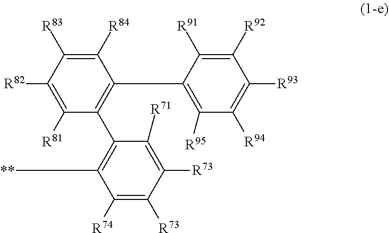

In the formula (1-e),
R⁷¹ to R⁷⁴, R⁸¹ to R⁸⁴, and R⁹¹ to R⁹⁵ are each independently a hydrogen atom, an unsubstituted alkyl group having 1 to 6 carbon atoms, or an unsubstituted phenyl group,
provided that,
adjacent two selected from R⁷¹ to R⁷⁴, R⁸¹ to R⁸⁴, and R⁹¹ to R⁹⁵ are not bonded to each other, and thus do not form a ring structure; and
** represents a bonding site to L²,
provided that, when L² is a single bond, ** represents a bonding site to the central nitrogen atom N*.

The unsubstituted alkyl groups having 1 to 6 carbon atoms represented by R⁷¹ to R⁷⁴, R⁸¹ to R⁸⁴, and R⁹¹ to R⁹⁵ are as described above for R⁴¹ to R⁴⁵, and preferred groups and the like are also the same.
R⁷¹ to R⁷⁴, R⁸¹ to R⁸⁴, and R⁹¹ to R⁹⁵ are each preferably a hydrogen atom.
Ar¹ is preferably a group represented by any one of the above formulae (1-a) to (1-c).

In the substituted or unsubstituted aryl group represented by Ar², which is composed of only a 6-membered ring having 6 to 30 ring carbon atoms, examples of the unsubstituted aryl group include a phenyl group, a biphenylyl group, a terphenylyl group, a naphthyl group, an anthryl group, a phenanthryl group, a phenalenyl group, a picenyl group, a pentaphenyl group, a pyrenyl group, a chrysenyl group, a perylenyl group, and a triphenylenyl group. Among these, a phenyl group, a biphenylyl group, or a naphthyl group is preferable.

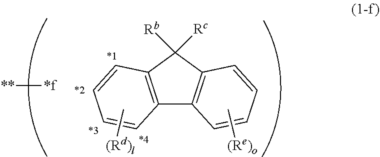

In the formula (1-f),
R^{b} and R^{c} are each independently an unsubstituted alkyl group having 1 to 6 carbon atoms, or an unsubstituted aryl group having 6 to 12 ring carbon atoms,
provided that,
when R^{b} and R^{c} are each an unsubstituted alkyl group having 1 to 6 carbon atoms, R^{b} and R^{c} are not bonded to each other and thus do not form a ring structure, and
when R^{b} and R^{c} are each an unsubstituted aryl group having 6 to 12 ring carbon atoms, R^{b} and R^{c} may be bonded to each other to form a substituted or unsubstituted ring;
R^{d} and R^{e} are each independently an unsubstituted aryl group having 6 to 12 ring carbon atoms;
l and o are each independently an integer of 0 to 2, in a case where 1 is 0, R^{d} is not present, and in a case where o is 0, R^{e} is not present;
*f is bonded to one carbon atom selected from carbon atoms *1 to *4 and a carbon atom contained in an unsubstituted alkyl group having 1 to 6 carbon atoms or an unsubstituted aryl group having 6 to 12 ring carbon atoms represented by R^{b} and R^{c}; and
*** represents a bonding site to L³,
provided that, when L³ is a single bond, *** represents a bonding site to the central nitrogen atom N*.

The unsubstituted alkyl group having 1 to 6 carbon atoms represented by R^{b} and R^{c} is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, still more preferably a methyl group or a t-butyl group, and even more preferably a methyl group.

The unsubstituted aryl group having 6 to 12 ring carbon atoms represented by R^{b} and R^{c} is as described above for R¹¹ to R¹⁸, and preferred groups and the like are also the same.

R^{b} and R^{c} are preferably a methyl group or a phenyl group.

The unsubstituted aryl group having 6 to 12 ring carbon atoms represented by R^{d} and R^{e} is as described above for R¹¹ to R¹⁸, and preferred groups and the like are also the same.

l and o are preferably 0 or 1, and more preferably 0.
* f is preferably bonded to a carbon atom *1, *2, *4, or a carbon atom contained in an unsubstituted alkyl group having 1 to 6 carbon atoms or an unsubstituted aryl group having 6 to 12 ring carbon atoms represented by R^{b} and R^{c}, and more preferably bonded to *2.

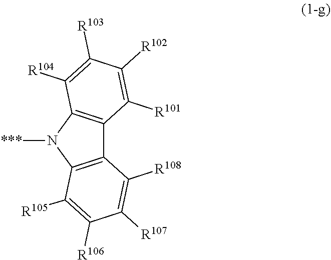

In the formula (1-g),
R¹⁰¹ to R¹⁰⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms,
provided that,
adjacent two selected from R¹⁰¹ to R¹⁰⁸ may be bonded to each other to form a substituted or unsubstituted benzene ring; and
*** represents a bonding site to L³,
provided that, when L³ is a single bond, *** represents a bonding site to the central nitrogen atom N*.

The substituted or unsubstituted alkyl group having 1 to 6 carbon atoms represented by R¹⁰¹ to R¹⁰⁸ is as described above for R¹¹ to R¹⁸, and preferred groups and the like are also the same.

The substituted or unsubstituted aromatic heterocyclic group having 5 to 13 ring atoms represented by R¹⁰¹ to R¹⁰⁸ is preferably a dibenzothiophenyl group, a dibenzofuranyl group, or a carbazolyl group.

Each of R¹⁰¹ to R¹⁰⁸, which is not a single bond, is preferably a hydrogen atom.

Ar² is preferably a group represented by any one of the following formulae (1-h) to (1-j).

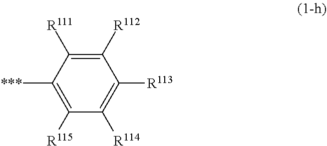

In the formula (1-h),
R¹¹¹ to R¹¹⁵ are each independently a hydrogen atom, a substituted or unsubstituted alkyl group having 1 to 6 carbon atoms, or a substituted or unsubstituted aryl group having 6 to 12 ring carbon atoms,
provided that,
adjacent two selected from R¹¹¹ to R¹¹⁵ are not bonded to each other, and thus do not form a ring structure; and
*** represents a bonding site to the central nitrogen atom N*.

The substituted or unsubstituted alkyl group having 1 to 6 carbon atoms represented by R¹¹¹ to R¹¹⁵ is as described above for R¹¹ to R¹⁸, and preferred groups and the like are also the same.

The unsubstituted aryl group having 6 to 12 ring carbon atoms represented by R¹¹¹ to R¹¹⁵ is as described above for R¹¹ to R¹⁸, and preferred groups and the like are also the same.

R¹¹¹ to R¹¹⁵ are each preferably a hydrogen atom.

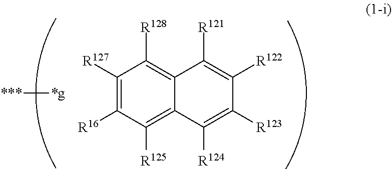

In the formula (1-i),
R¹²¹ to R¹²⁸ are each independently a hydrogen atom, a substituted or unsubstituted alkyl group having 1 to 6 carbon atoms, or a substituted or unsubstituted aryl group having 6 to 12 ring carbon atoms,
provided that,
one selected from R¹²¹ to R¹²⁸ is a single bond bonded to *g, and
adjacent two selected from R¹²¹ to R¹²⁸, which are not a single bond, are not bonded to each other, and thus do not form a ring structure; and
*** represents a bonding site to the central nitrogen atom N*.

The substituted or unsubstituted alkyl group having 1 to 6 carbon atoms represented by R¹²¹ to R¹²⁸ is as described above for R¹¹ to R¹⁸, and preferred groups and the like are also the same.

The unsubstituted aryl group having 6 to 12 ring carbon atoms represented by R¹²¹ to R¹²⁸ is as described above for R¹¹ to R¹⁸, and preferred groups and the like are also the same.

Each of R¹²¹ to R¹²⁸, which is not a single bond, is preferably a hydrogen atom.

In one embodiment of the present invention, one selected from R¹²¹, R¹²⁴, R¹²⁵, and R¹²⁸ is preferably a single bond bonded to *h.

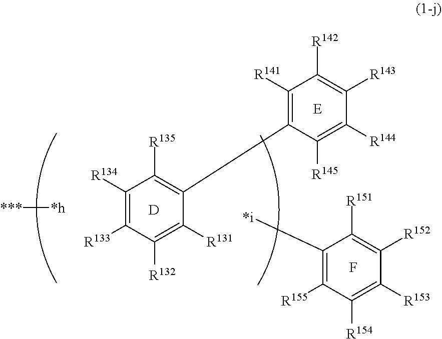

In the formula (1-j),
R¹³¹ to R¹³⁵ are each independently a hydrogen atom, an unsubstituted alkyl group having 1 to 6 carbon atoms, or an unsubstituted phenyl group;
R¹⁴¹ to R¹⁴⁵ and R¹⁵¹ to R¹⁵⁵ are each independently a hydrogen atom or an unsubstituted alkyl group having 1 to 6 carbon atoms,
provided that,
one selected from R¹³¹ to R¹³⁵ is a single bond bonded to *h,
another one selected from R¹³¹ to R¹³⁵ is a single bond bonded to *i,
adjacent two selected from R¹³¹ to R¹³⁵, which are not a single bond, are not bonded to each other, and thus do not form a ring structure, and
adjacent two selected from R¹⁴¹ to R¹⁴⁵ and R¹⁵¹ to R¹⁵⁵ may be bonded to each other to form a substituted or unsubstituted benzene ring; and
*** represents a bonding site to the central nitrogen atom N*.

The unsubstituted alkyl group having 1 to 6 carbon atoms represented by R¹³¹ to R¹³⁵ is as described above for R⁴¹ to R⁴⁵, and preferred groups and the like are also the same.

Each of R¹³¹ to R¹³⁵, which is not a single bond, is preferably a hydrogen atom.

The unsubstituted alkyl groups having 1 to 6 carbon atoms represented by R¹⁴¹ to R¹⁴⁵ and R¹⁵¹ to R¹⁵⁵ are as described above for R⁴¹ to R⁴⁵, and preferred groups and the like are also the same.

Each of R¹⁴¹ to R¹⁴⁵ and R¹⁵¹ to R¹⁵⁵, which is not a single bond, is preferably a hydrogen atom.

It is preferable that Ar² is a group represented by any one of the above formulae (1-h) to (1-j), and it is more preferable that Ar¹ is a group represented by any one of the above formulae (1-a) to (1-c), and Ar² is a group represented by any one of the above formulae (1-h) to (1-j).

In one embodiment of the present invention,
(A-1) all of R¹¹ to R¹⁸ which are not a single bond bonded to *a may be a hydrogen atom;
(A-2) all of R²¹ to R³⁰ which are not a single bond bonded to *b may be a hydrogen atom;
(A-3) all of R³¹ to R³⁸ which are not a single bond bonded to *c may be a hydrogen atom;
(A-4) all of R⁴¹ to R⁴⁵ which are not a single bond bonded to *d and *e may be a hydrogen atom;
(A-5) all of R⁵¹ to R⁵⁵ and R⁶¹ to R⁶⁵ may be a hydrogen atom;
(A-6) all of R⁷¹ to R⁷⁴, R⁸¹ to R⁸⁴, and R⁹¹ to R⁹⁵ may be a hydrogen atom;
(A-7) all of R¹⁰¹ to R¹⁰⁸ may be a hydrogen atom;
(A-8) all of R¹¹¹ to R¹¹⁵ may be a hydrogen atom;
(A-9) all of R¹²¹ to R¹²⁸ which are not a single bond bonded to *g may be a hydrogen atom;
(A-10) all of R¹³¹ to R¹³⁵ which are not a single bond bonded to *h and *i may be a hydrogen atom; and
(A-11) all of R¹⁴¹ to R¹⁴⁵ and R¹⁵¹ to R¹⁵⁵ may be a hydrogen atom.

As described above, the “hydrogen atom” used in the description herein includes a protium atom, a deuterium atom, and a tritium atom. Accordingly, the inventive compound may contain a naturally-derived deuterium atom.

A deuterium atom may be intentionally introduced into the inventive compound A by using a deuterated compound as a part or the whole of the raw material compound. Thus, in one embodiment of the present invention, the inventive compound contains at least one deuterium atom. That is, the inventive compound may be a compound represented by the formula (1) in which at least one hydrogen atom contained in the compound is a deuterium atom.

At least one hydrogen atom selected from the following hydrogen atoms may be a deuterium atom:
a hydrogen atom represented by any one of R¹ to R⁹;
a hydrogen atom contained in an unsubstituted arylene group represented by L¹, L², and L³;
a hydrogen atom contained in an aryl group composed of only a substituted or unsubstituted 6-membered ring represented by Ar²;
a hydrogen atom represented by any one of R¹¹ to R¹⁸ which are not a single bond; a hydrogen atom contained in a substituted or unsubstituted alkyl group or aryl group represented by any one of R¹¹ to R¹⁸ which are not a single bond;
a hydrogen atom represented by any one of R²¹ to R³⁰ which are not a single bond; a hydrogen atom contained in a substituted or unsubstituted alkyl group or aryl group represented by any one of R²¹ to R³⁰ which are not a single bond;
a hydrogen atom represented by any one of R³¹ to R³⁸ which are not a single bond; a hydrogen atom contained in a substituted or unsubstituted alkyl group, aryl group, or aromatic heterocyclic group represented by any one of R³¹ to R³⁸ which are not a single bond;
a hydrogen atom represented by R^{a} which is not a single bond; a hydrogen atom contained in a substituted or unsubstituted alkyl group, aryl group, or aromatic heterocyclic group represented by R^{a};
a hydrogen atom represented by any one of R⁴¹ to R⁴⁵ which are not a single bond; a hydrogen atom contained in an unsubstituted alkyl group or a phenyl group represented by any one of R⁴¹ to R⁴⁵ which are not a single bond;
a hydrogen atom represented by any one of R⁵¹ to R⁵⁵ and R⁶¹ to R⁶⁵; a hydrogen atom contained in an unsubstituted alkyl group represented by any one of R⁵¹ to R⁵⁵ and R⁶¹ to R⁶⁵;
a hydrogen atom represented by any one of R⁷¹ to R⁷⁴, R⁸¹ to R⁸⁴, and R⁹¹ to R⁹⁵; a hydrogen atom contained in an unsubstituted alkyl group or a phenyl group represented by any one of R⁷¹ to R⁷⁴, R⁸¹ to R⁸⁴, and R⁹¹ to R⁹⁵;
a hydrogen atom contained in an unsubstituted alkyl group or aryl group represented by any one of R^{b} and R^{c};
a hydrogen atom represented by any one of R¹⁰¹ to R¹⁰⁸ which are not a single bond;
a hydrogen atom contained in a substituted or unsubstituted alkyl group, aryl group, or aromatic heterocyclic group represented by any one of R¹⁰¹ to R¹⁰⁸ which are not a single bond;
a hydrogen atom represented by any one of R¹¹¹ to R¹¹⁵; a hydrogen atom contained in a substituted or unsubstituted alkyl group or aryl group represented by any one of R¹¹¹ to R¹¹⁵;
a hydrogen atom represented by any one of R¹²¹ to R¹²⁸ which are not a single bond;
a hydrogen atom contained in a substituted or unsubstituted alkyl group or aryl group represented by any one of R¹²¹ to R¹²⁸ which are not a single bond;
a hydrogen atom represented by any one of R¹³¹ to R¹³⁵ which are not a single bond;
a hydrogen atom contained in an unsubstituted alkyl group or a phenyl group represented by any one of R¹³¹ to R¹³⁵ which are not a single bond;
a hydrogen atom represented by any one of R¹⁴¹ to R¹⁴⁵ and R¹⁵¹ to R¹⁵⁵; a hydrogen atom contained in an unsubstituted alkyl group represented by any one of R¹⁴¹ to R¹⁴⁵ and R¹⁵¹ to R¹⁵⁵; and
a hydrogen atom represented by any one of R¹⁶¹ to R¹⁶⁴ and R¹⁷¹ to R¹⁷⁴.

The deuteration rate of the inventive compound depends on the deuteration rate of the raw material compound used. Even when a raw material having a predetermined deuteration rate is used, a naturally-derived protium isotope can be contained in a certain ratio. Accordingly, an embodiment of the deuteration rate of the inventive compound shown below includes the proportion for which a minor amount of a naturally-derived isotope is taken into consideration, relative to the proportion determined by counting the number of the deuterium atoms merely represented by a chemical formula.

The deuteration rate of the inventive compound is preferably 1% or more, more preferably 3% or more, still more preferably 5% or more, even more preferably 10% or more, and even more preferably 50% or more.

The inventive compound may be a mixture of a deuterated compound and a non-deuterated compound, or a mixture of two or more compounds having different deuteration rates from each other. The deuteration rate of the mixture is preferably 1% or more, more preferably 3% or more, still more preferably 5% or more, even more preferably 10% or more, even more preferably 50% or more, and is less than 100%. The proportion of the number of the deuterium atoms to the number of all the hydrogen atoms in the inventive compound is preferably 1% or more, more preferably 3% or more, still more preferably 5% or more, even more preferably 10% or more, and is 100% or less.

The details of the substituent (arbitrary substituent) in the case of “substituted or unsubstituted” included in the definition of each of the above formulae are as described in “Substituent for “Substituted or Unsubstituted””.

The inventive compound can be readily produced by a person skilled in the art with reference to the following synthesis examples and the known synthesis methods.

Specific examples of the inventive compound will be described below, but the inventive compound is not limited to the following example compounds.

In the following specific examples, D represents a deuterium atom.

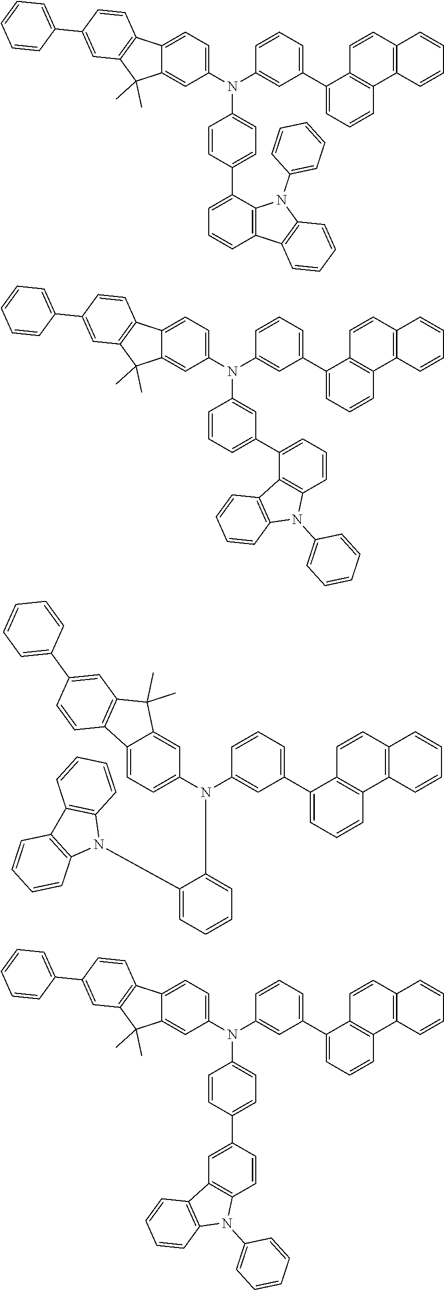 [SMILES: CC1(C)c2cc(-c3ccccc3)ccc2-c2ccc(N(c3ccc(-c4cccc5c6ccccc6n(-c6ccccc6)c45)cc3)c3cccc(-c4cccc5c4ccc4ccccc45)c3)cc21 ; CC1(C)c2cc(-c3ccccc3)ccc2-c2ccc(N(c3cccc(-c4cccc5c4ccc4ccccc45)c3)c3cccc(-c4cccc5c4c4ccccc4n5-c4ccccc4)c3)cc21 ; CC1(C)c2cc(-c3ccccc3)ccc2-c2ccc(N(c3cccc(-c4cccc5c4ccc4ccccc45)c3)c3ccccc3-n3c4ccccc4c4ccccc43)cc21 ; CC1(C)c2cc(-c3ccccc3)ccc2-c2ccc(N(c3ccc(-c4ccc5c(c4)c4ccccc4n5-c4ccccc4)cc3)c3cccc(-c4cccc5c4ccc4ccccc45)c3)cc21]

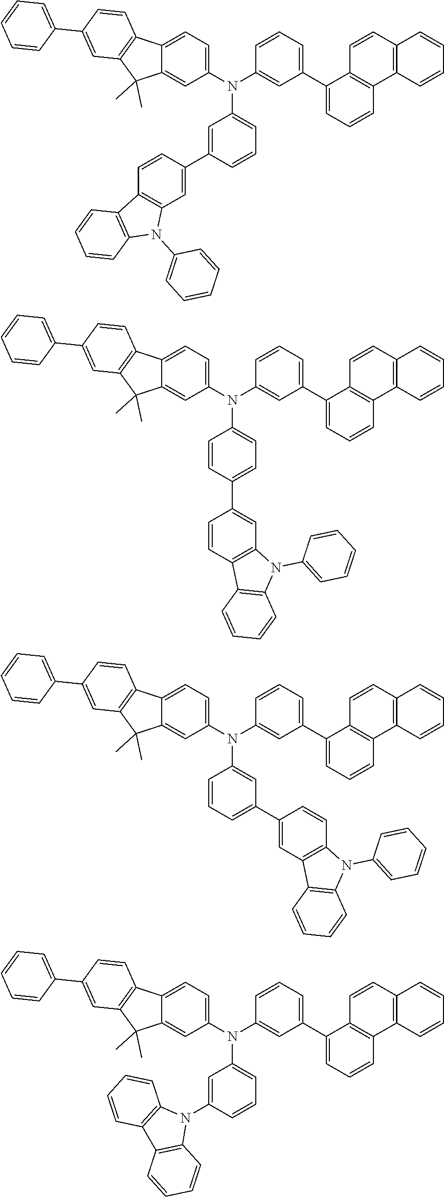 [SMILES: CC1(C)c2cc(-c3ccccc3)ccc2-c2ccc(N(c3cccc(-c4ccc5c6ccccc6n(-c6ccccc6)c5c4)c3)c3cccc(-c4cccc5c4ccc4ccccc45)c3)cc21 ; CC1(C)c2cc(-c3ccccc3)ccc2-c2ccc(N(c3ccc(-c4ccc5c6ccccc6n(-c6ccccc6)c5c4)cc3)c3cccc(-c4cccc5c4ccc4ccccc45)c3)cc21 ; CC1(C)c2cc(-c3ccccc3)ccc2-c2ccc(N(c3cccc(-c4ccc5c(c4)c4ccccc4n5-c4ccccc4)c3)c3cccc(-c4cccc5c4ccc4ccccc45)c3)cc21 ; CC1(C)c2cc(-c3ccccc3)ccc2-c2ccc(N(c3cccc(-c4cccc5c4ccc4ccccc45)c3)c3cccc(-n4c5ccccc5c5ccccc54)c3)cc21]

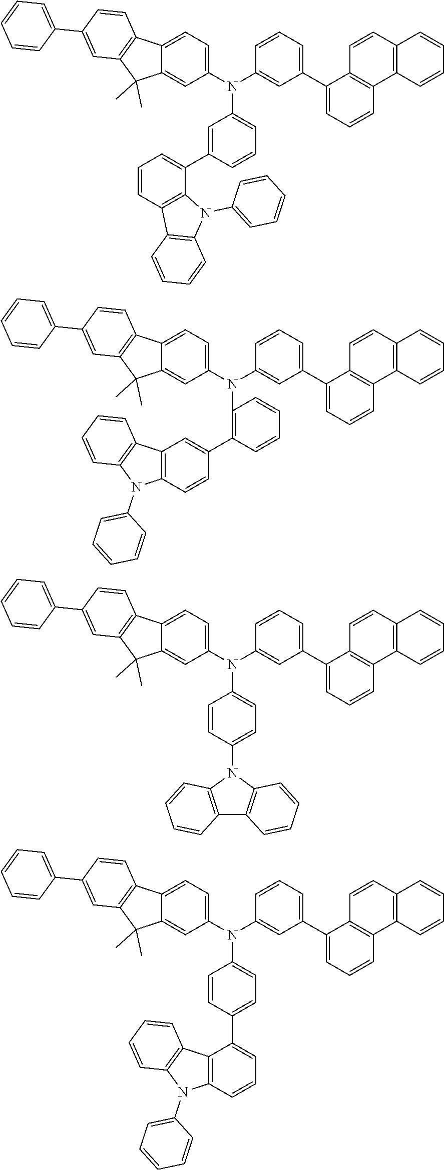

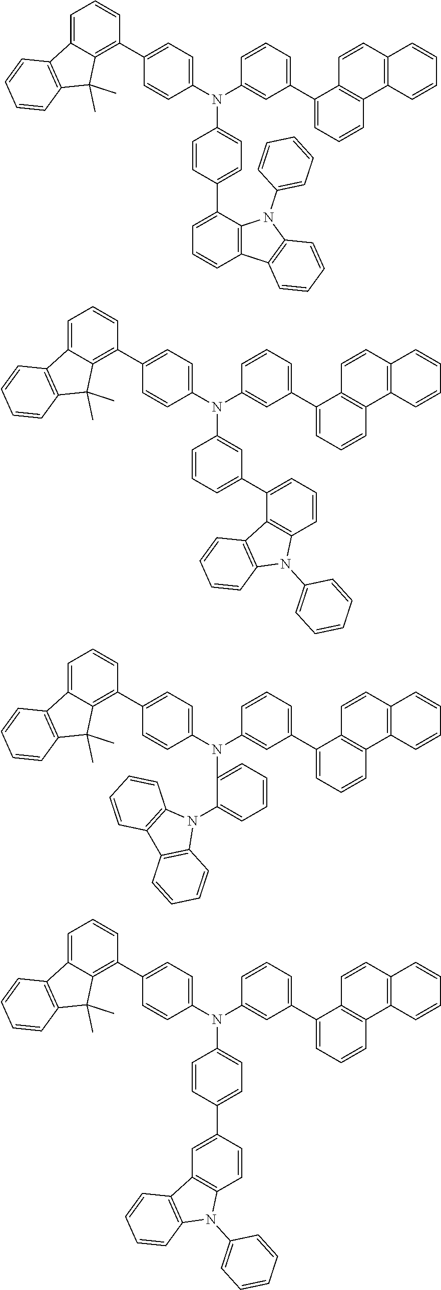 [SMILES: CC1(C)c2ccccc2-c2cccc(-c3ccc(N(c4ccc(-c5cccc6c7ccccc7n(-c7ccccc7)c56)cc4)c4cccc(-c5cccc6c5ccc5ccccc56)c4)cc3)c21 ; CC1(C)c2ccccc2-c2cccc(-c3ccc(N(c4cccc(-c5cccc6c5ccc5ccccc56)c4)c4cccc(-c5cccc6c5c5ccccc5n6-c5ccccc5)c4)cc3)c21 ; CC1(C)c2ccccc2-c2cccc(-c3ccc(N(c4cccc(-c5cccc6c5ccc5ccccc56)c4)c4ccccc4-n4c5ccccc5c5ccccc54)cc3)c21 ; CC1(C)c2ccccc2-c2cccc(-c3ccc(N(c4ccc(-c5ccc6c(c5)c5ccccc5n6-c5ccccc5)cc4)c4cccc(-c5cccc6c5ccc5ccccc56)c4)cc3)c21]

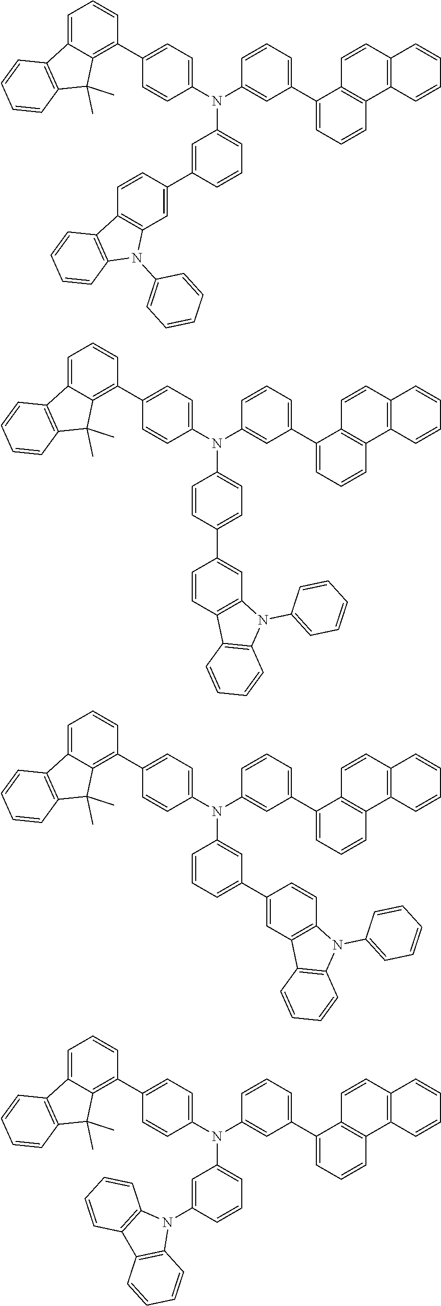 [SMILES: CC1(C)c2ccccc2-c2cccc(-c3ccc(N(c4cccc(-c5ccc6c7ccccc7n(-c7ccccc7)c6c5)c4)c4cccc(-c5cccc6c5ccc5ccccc56)c4)cc3)c21 ; CC1(C)c2ccccc2-c2cccc(-c3ccc(N(c4ccc(-c5ccc6c7ccccc7n(-c7ccccc7)c6c5)cc4)c4cccc(-c5cccc6c5ccc5ccccc56)c4)cc3)c21 ; CC1(C)c2ccccc2-c2cccc(-c3ccc(N(c4cccc(-c5ccc6c(c5)c5ccccc5n6-c5ccccc5)c4)c4cccc(-c5cccc6c5ccc5ccccc56)c4)cc3)c21 ; CC1(C)c2ccccc2-c2cccc(-c3ccc(N(c4cccc(-c5cccc6c5ccc5ccccc56)c4)c4cccc(-n5c6ccccc6c6ccccc65)c4)cc3)c21]

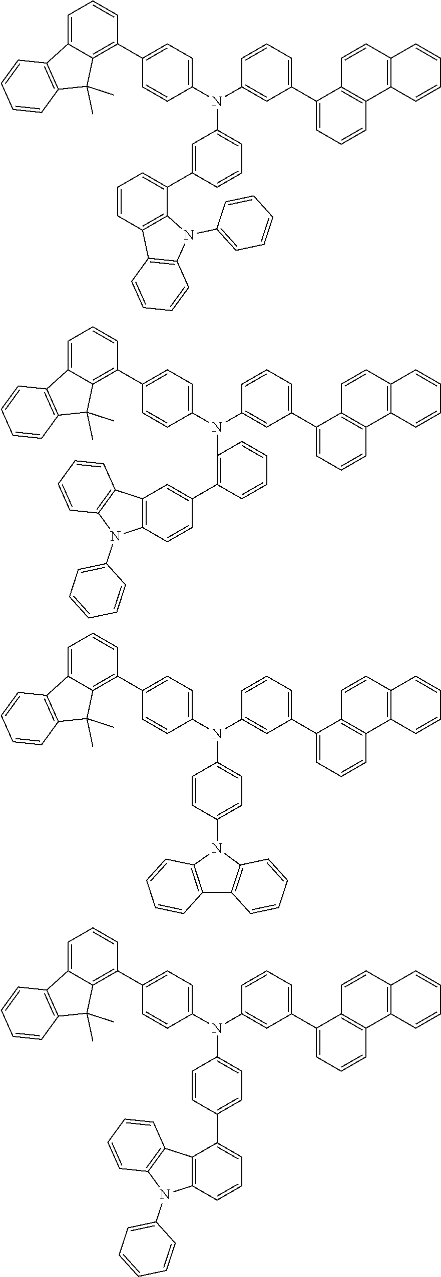

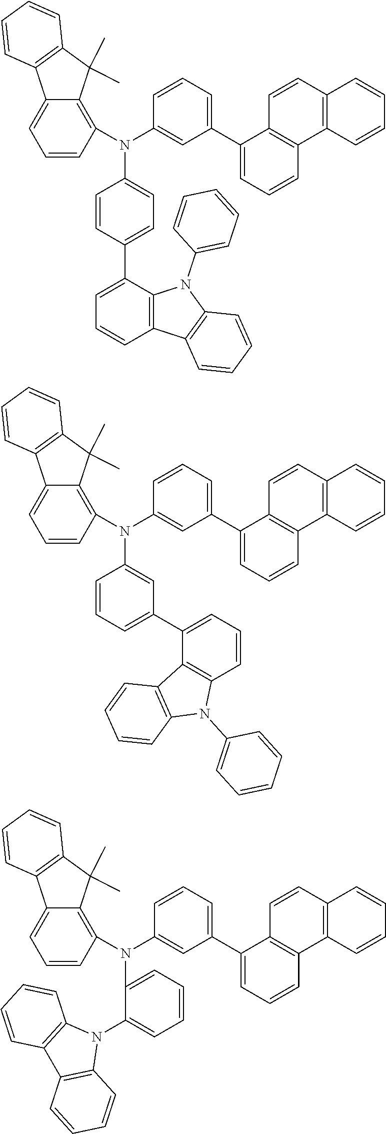 [SMILES: CC1(C)c2ccccc2-c2cccc(N(c3ccc(-c4cccc5c6ccccc6n(-c6ccccc6)c45)cc3)c3cccc(-c4cccc5c4ccc4ccccc45)c3)c21 ; CC1(C)c2ccccc2-c2cccc(N(c3cccc(-c4cccc5c4ccc4ccccc45)c3)c3cccc(-c4cccc5c4c4ccccc4n5-c4ccccc4)c3)c21 ; CC1(C)c2ccccc2-c2cccc(N(c3cccc(-c4cccc5c4ccc4ccccc45)c3)c3ccccc3-n3c4ccccc4c4ccccc43)c21]

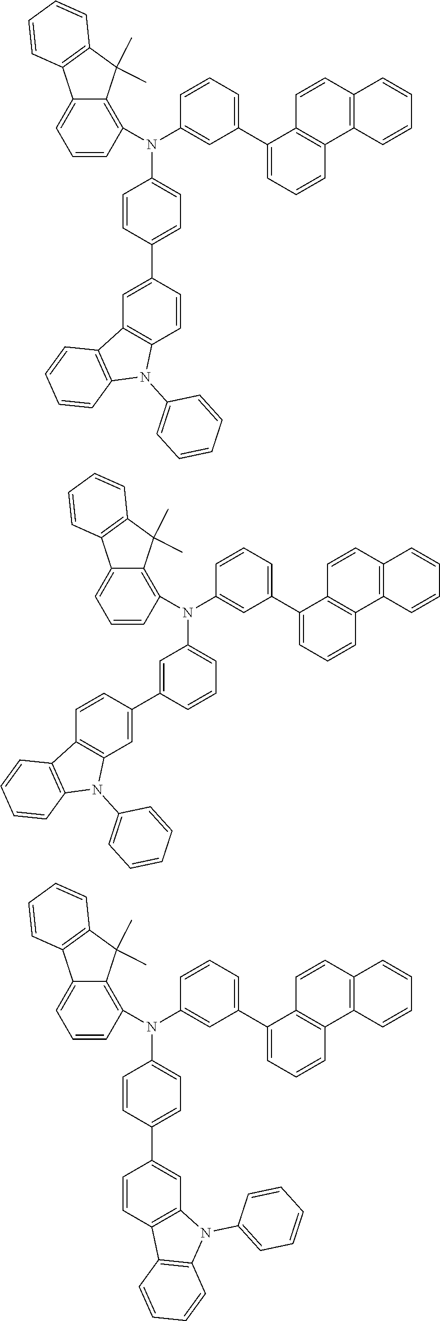 [SMILES: CC1(C)c2ccccc2-c2cccc(N(c3ccc(-c4ccc5c(c4)c4ccccc4n5-c4ccccc4)cc3)c3cccc(-c4cccc5c4ccc4ccccc45)c3)c21 ; CC1(C)c2ccccc2-c2cccc(N(c3cccc(-c4ccc5c6ccccc6n(-c6ccccc6)c5c4)c3)c3cccc(-c4cccc5c4ccc4ccccc45)c3)c21 ; CC1(C)c2ccccc2-c2cccc(N(c3ccc(-c4ccc5c6ccccc6n(-c6ccccc6)c5c4)cc3)c3cccc(-c4cccc5c4ccc4ccccc45)c3)c21]

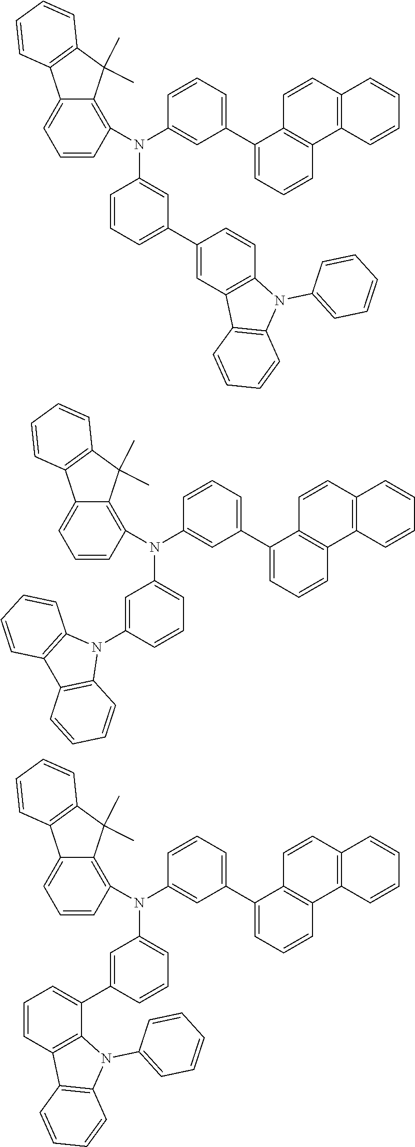

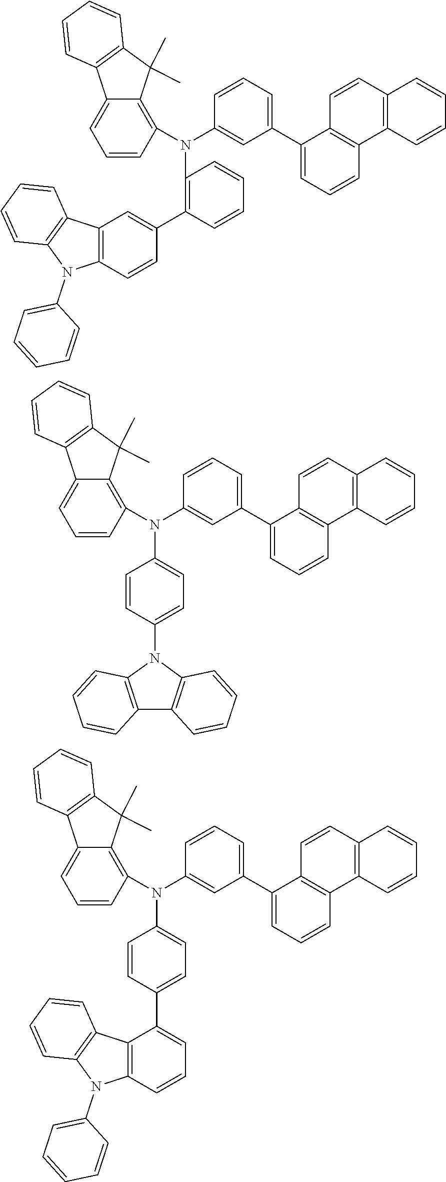

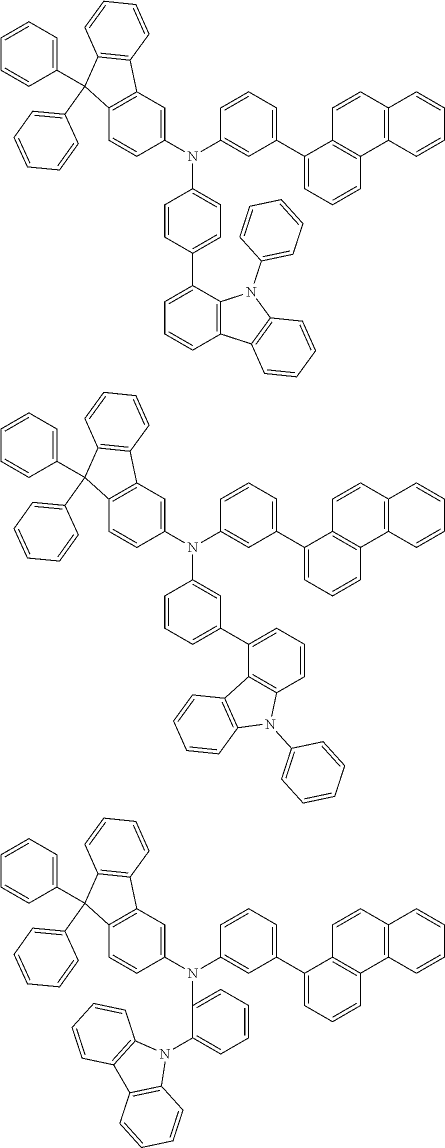 [SMILES: c1ccc(-n2c3ccccc3c3cccc(-c4ccc(N(c5cccc(-c6cccc7c6ccc6ccccc67)c5)c5ccc6c(c5)-c5ccccc5C6(c5ccccc5)c5ccccc5)cc4)c32)cc1 ; c1ccc(-n2c3ccccc3c3c(-c4cccc(N(c5cccc(-c6cccc7c6ccc6ccccc67)c5)c5ccc6c(c5)-c5ccccc5C6(c5ccccc5)c5ccccc5)c4)cccc32)cc1 ; c1ccc(C2(c3ccccc3)c3ccccc3-c3cc(N(c4cccc(-c5cccc6c5ccc5ccccc56)c4)c4ccccc4-n4c5ccccc5c5ccccc54)ccc32)cc1]

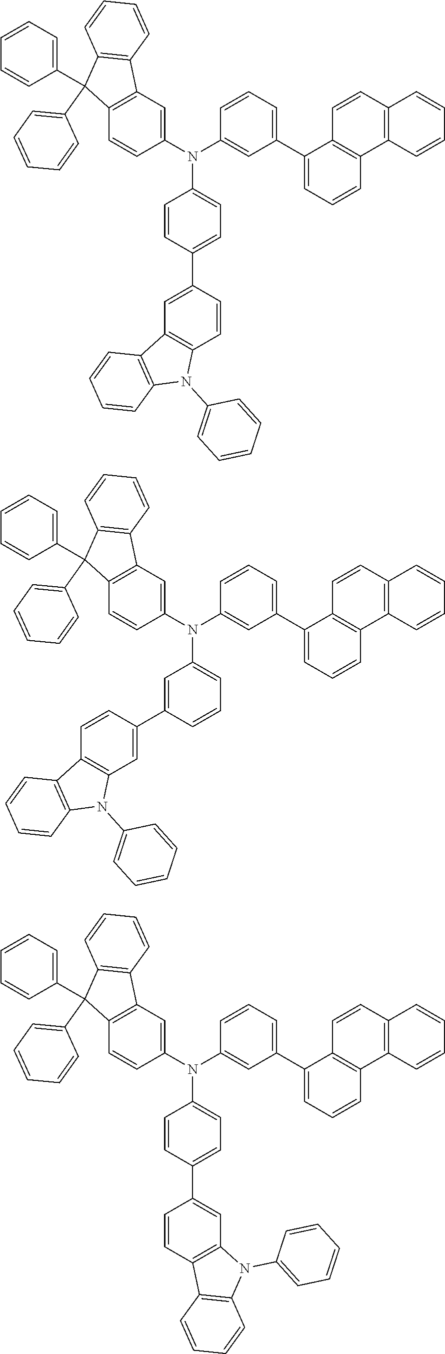

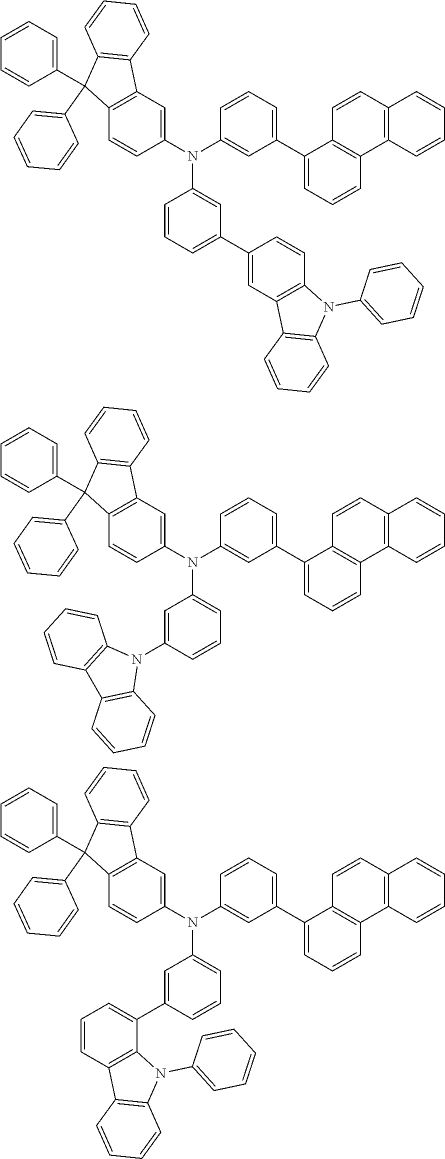

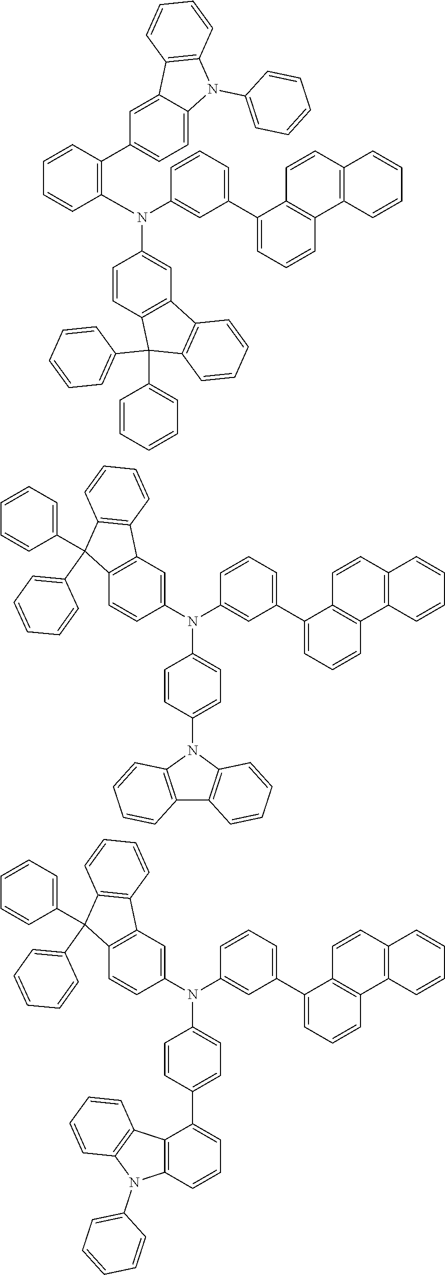

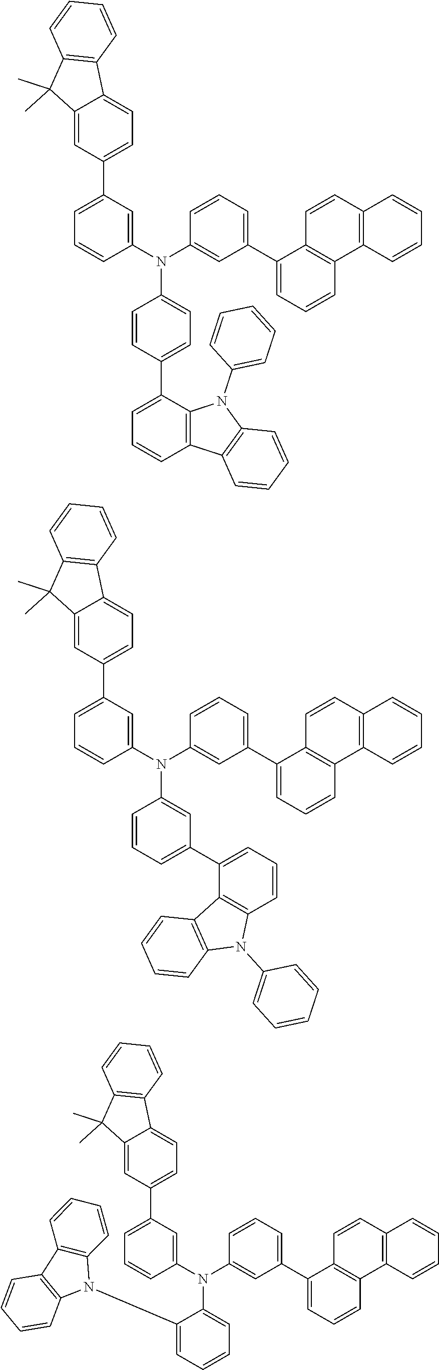 [SMILES: CC1(C)c2ccccc2-c2ccc(-c3cccc(N(c4ccc(-c5cccc6c7ccccc7n(-c7ccccc7)c56)cc4)c4cccc(-c5cccc6c5ccc5ccccc56)c4)c3)cc21 ; CC1(C)c2ccccc2-c2ccc(-c3cccc(N(c4cccc(-c5cccc6c5ccc5ccccc56)c4)c4cccc(-c5cccc6c5c5ccccc5n6-c5ccccc5)c4)c3)cc21 ; CC1(C)c2ccccc2-c2ccc(-c3cccc(N(c4cccc(-c5cccc6c5ccc5ccccc56)c4)c4ccccc4-n4c5ccccc5c5ccccc54)c3)cc21]

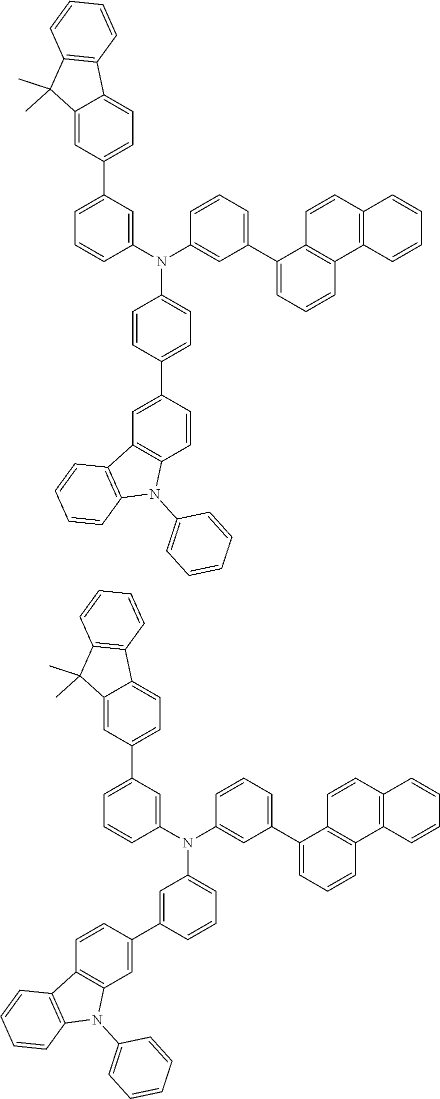 [SMILES: CC1(C)c2ccccc2-c2ccc(-c3cccc(N(c4ccc(-c5ccc6c(c5)c5ccccc5n6-c5ccccc5)cc4)c4cccc(-c5cccc6c5ccc5ccccc56)c4)c3)cc21 ; CC1(C)c2ccccc2-c2ccc(-c3cccc(N(c4cccc(-c5ccc6c7ccccc7n(-c7ccccc7)c6c5)c4)c4cccc(-c5cccc6c5ccc5ccccc56)c4)c3)cc21]

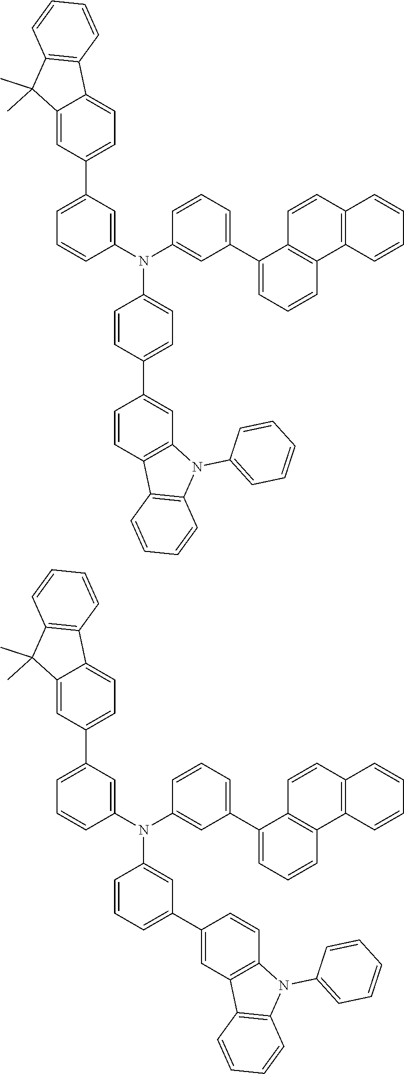 [SMILES: CC1(C)c2ccccc2-c2ccc(-c3cccc(N(c4ccc(-c5ccc6c7ccccc7n(-c7ccccc7)c6c5)cc4)c4cccc(-c5cccc6c5ccc5ccccc56)c4)c3)cc21 ; CC1(C)c2ccccc2-c2ccc(-c3cccc(N(c4cccc(-c5ccc6c(c5)c5ccccc5n6-c5ccccc5)c4)c4cccc(-c5cccc6c5ccc5ccccc56)c4)c3)cc21]

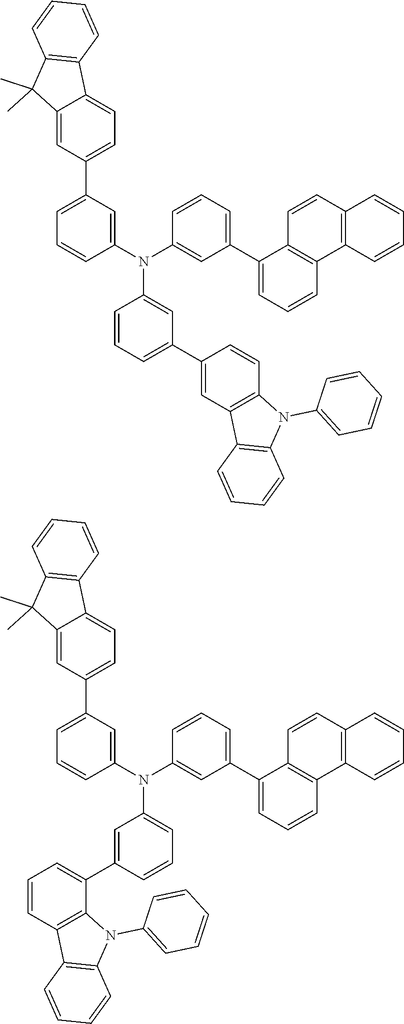 [SMILES: CC1(C)c2ccccc2-c2ccc(-c3cccc(N(c4cccc(-c5ccc6c(c5)c5ccccc5n6-c5ccccc5)c4)c4cccc(-c5cccc6c5ccc5ccccc56)c4)c3)cc21 ; CC1(C)c2ccccc2-c2ccc(-c3cccc(N(c4cccc(-c5cccc6c5ccc5ccccc56)c4)c4cccc(-c5cccc6c7ccccc7n(-c7ccccc7)c56)c4)c3)cc21]

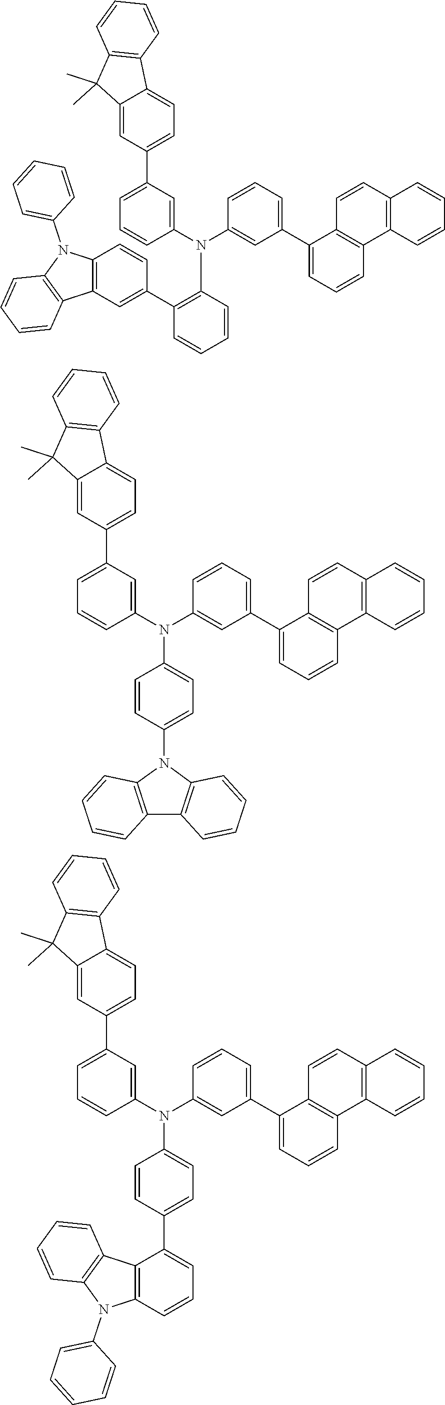

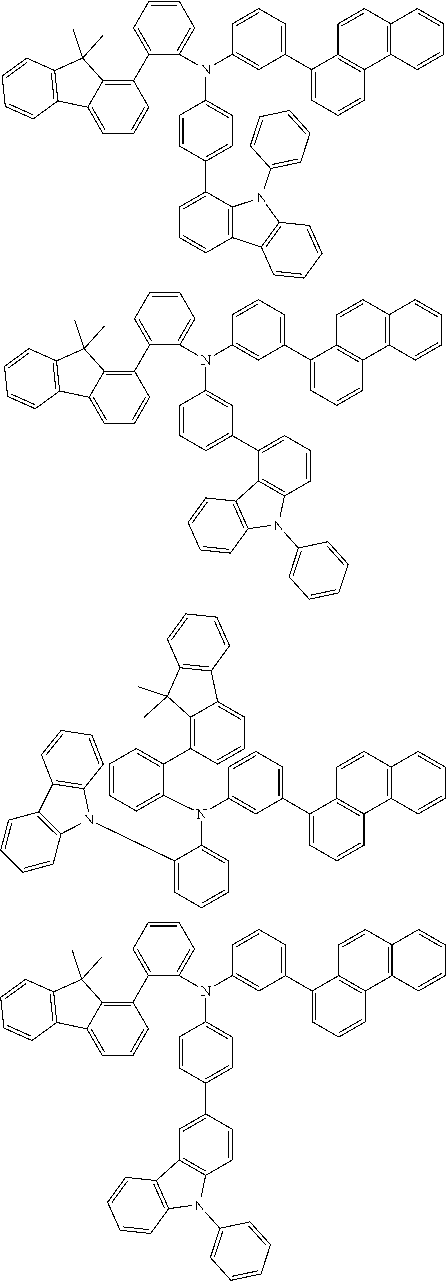

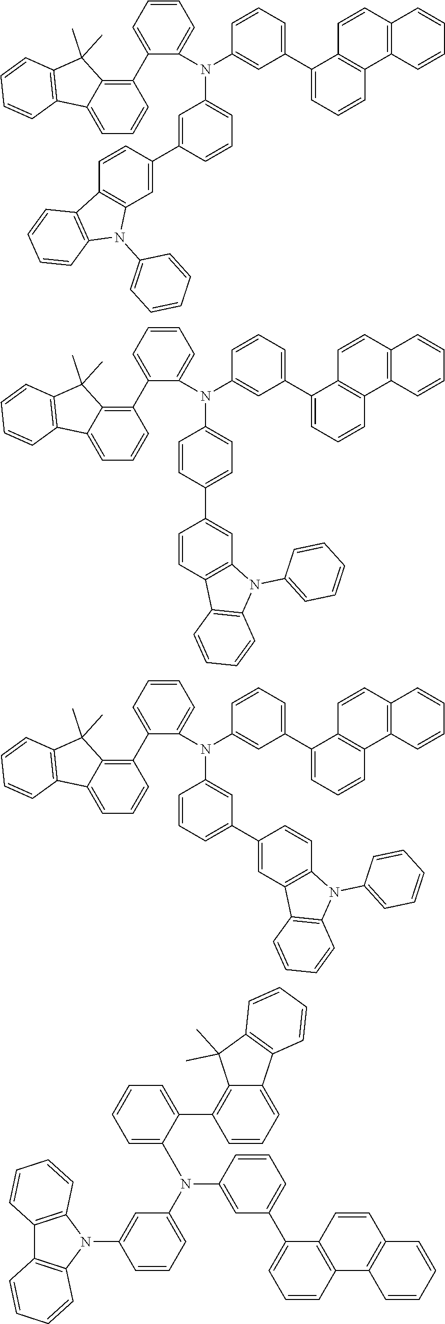

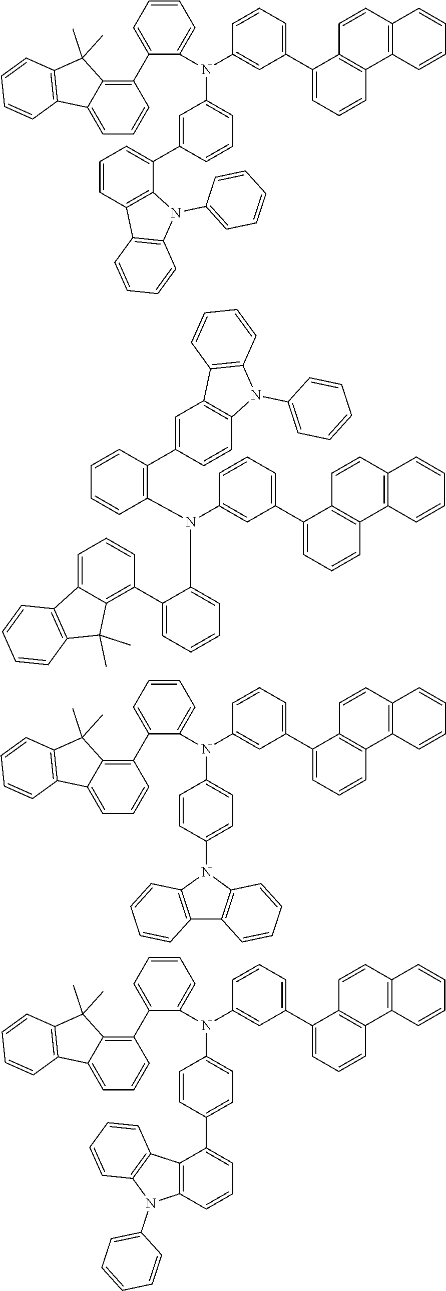

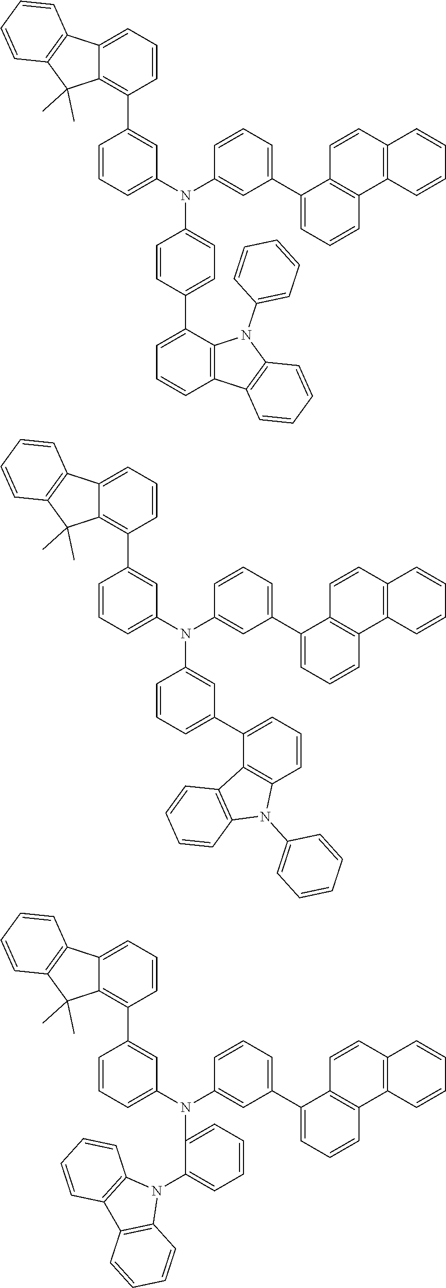 [SMILES: CC1(C)c2ccccc2-c2cccc(-c3cccc(N(c4ccc(-c5cccc6c7ccccc7n(-c7ccccc7)c56)cc4)c4cccc(-c5cccc6c5ccc5ccccc56)c4)c3)c21 ; CC1(C)c2ccccc2-c2cccc(-c3cccc(N(c4cccc(-c5cccc6c5ccc5ccccc56)c4)c4cccc(-c5cccc6c5c5ccccc5n6-c5ccccc5)c4)c3)c21 ; CC1(C)c2ccccc2-c2cccc(-c3cccc(N(c4cccc(-c5cccc6c5ccc5ccccc56)c4)c4ccccc4-n4c5ccccc5c5ccccc54)c3)c21]

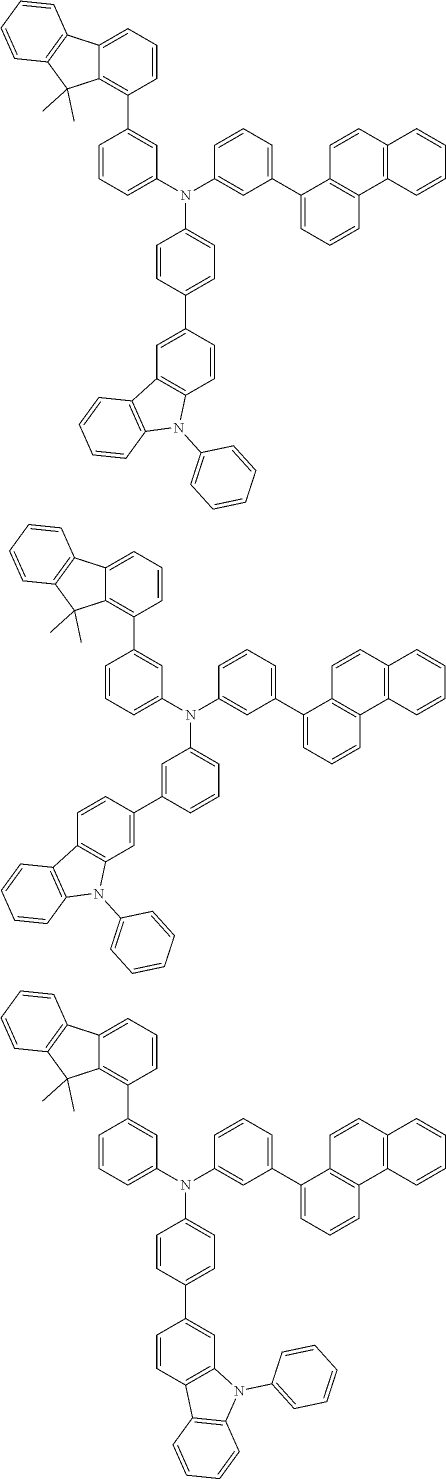

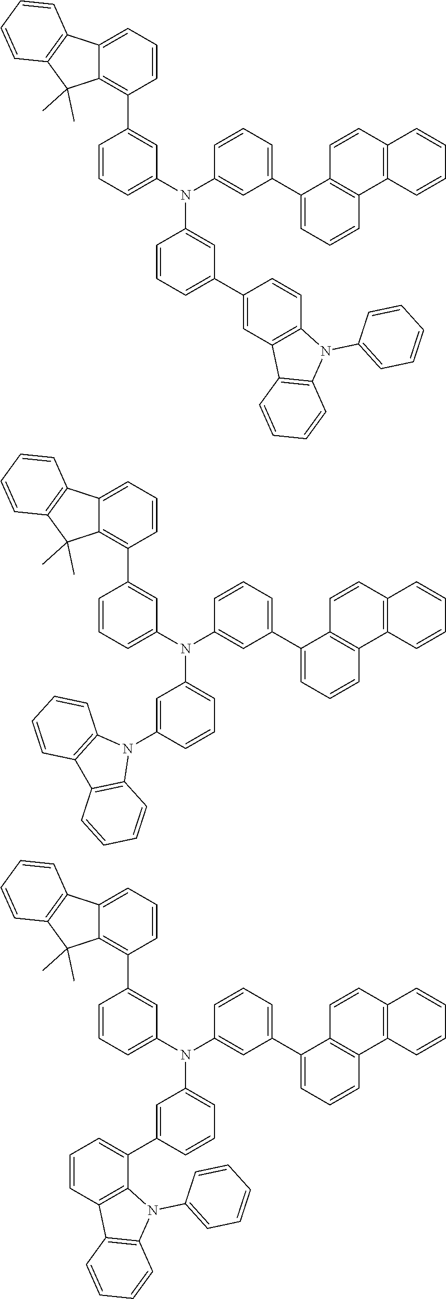 [SMILES: CC1(C)c2ccccc2-c2cccc(-c3cccc(N(c4cccc(-c5ccc6c(c5)c5ccccc5n6-c5ccccc5)c4)c4cccc(-c5cccc6c5ccc5ccccc56)c4)c3)c21 ; CC1(C)c2ccccc2-c2cccc(-c3cccc(N(c4cccc(-c5cccc6c5ccc5ccccc56)c4)c4cccc(-n5c6ccccc6c6ccccc65)c4)c3)c21 ; CC1(C)c2ccccc2-c2cccc(-c3cccc(N(c4cccc(-c5cccc6c5ccc5ccccc56)c4)c4cccc(-c5cccc6c7ccccc7n(-c7ccccc7)c56)c4)c3)c21]

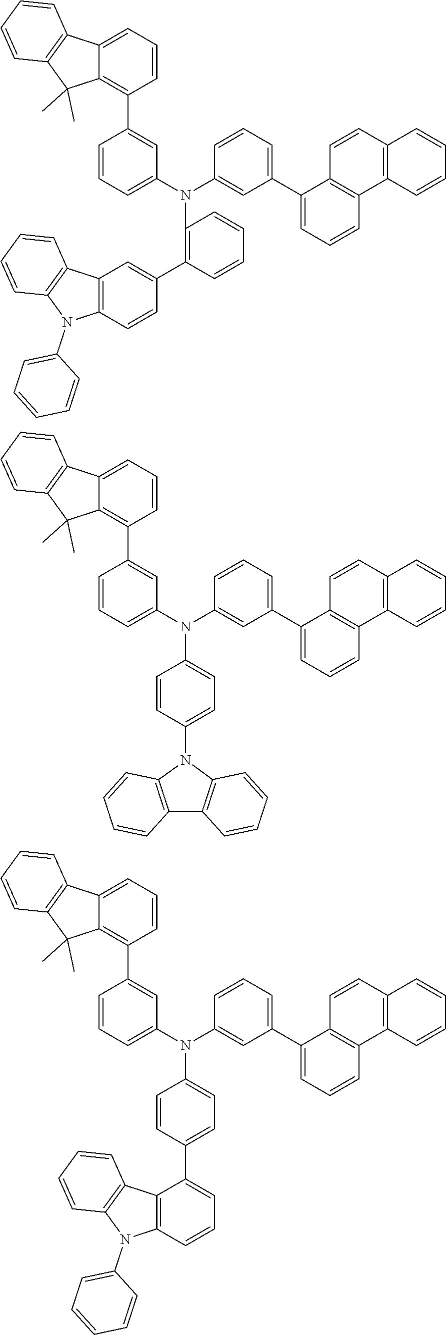

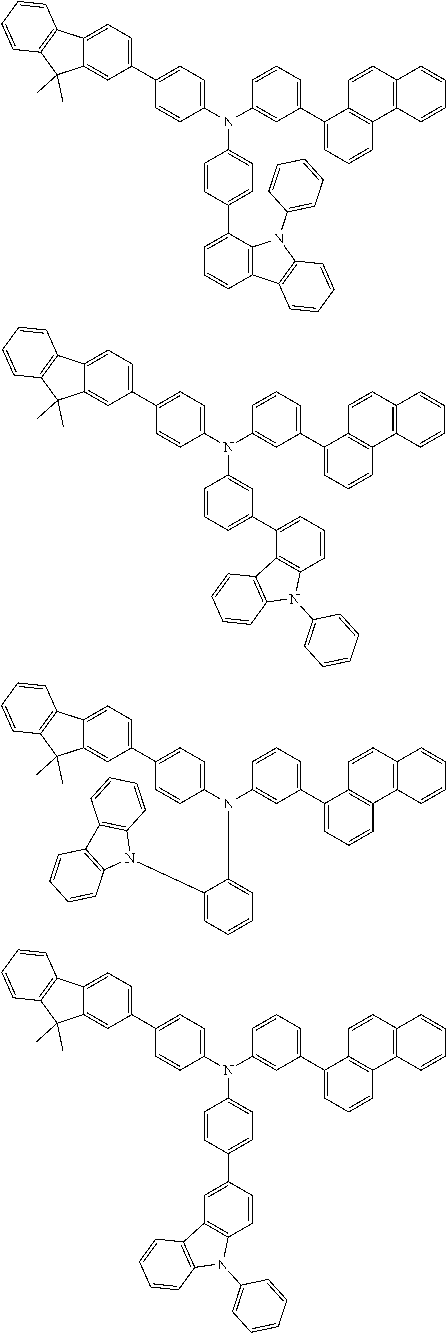 [SMILES: CC1(C)c2ccccc2-c2ccc(-c3ccc(N(c4ccc(-c5cccc6c7ccccc7n(-c7ccccc7)c56)cc4)c4cccc(-c5cccc6c5ccc5ccccc56)c4)cc3)cc21 ; CC1(C)c2ccccc2-c2ccc(-c3ccc(N(c4cccc(-c5cccc6c5ccc5ccccc56)c4)c4cccc(-c5cccc6c5c5ccccc5n6-c5ccccc5)c4)cc3)cc21 ; CC1(C)c2ccccc2-c2ccc(-c3ccc(N(c4cccc(-c5cccc6c5ccc5ccccc56)c4)c4ccccc4-n4c5ccccc5c5ccccc54)cc3)cc21 ; CC1(C)c2ccccc2-c2ccc(-c3ccc(N(c4ccc(-c5ccc6c(c5)c5ccccc5n6-c5ccccc5)cc4)c4cccc(-c5cccc6c5ccc5ccccc56)c4)cc3)cc21]

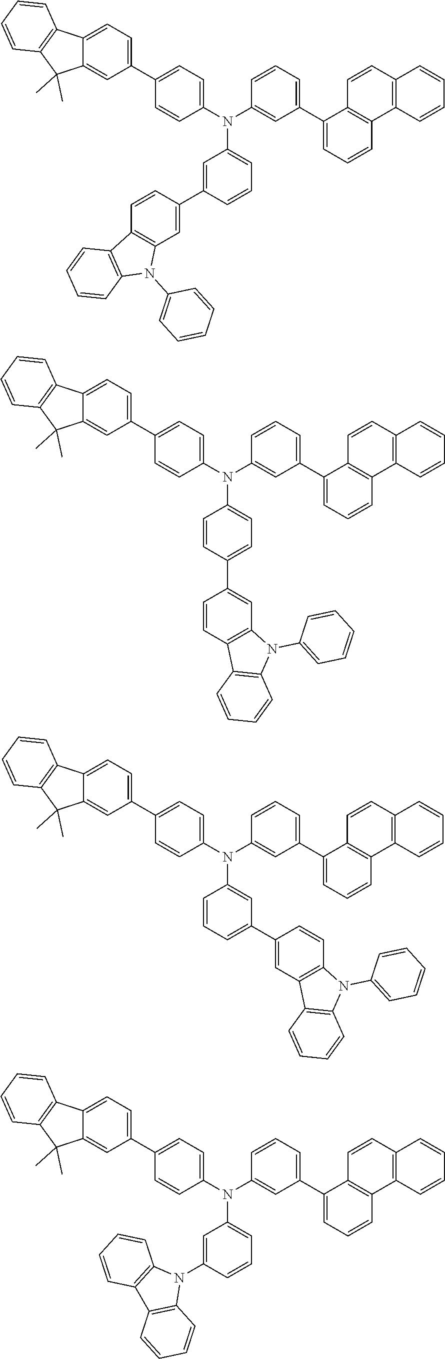 [SMILES: CC1(C)c2ccccc2-c2ccc(-c3ccc(N(c4cccc(-c5ccc6c7ccccc7n(-c7ccccc7)c6c5)c4)c4cccc(-c5cccc6c5ccc5ccccc56)c4)cc3)cc21 ; CC1(C)c2ccccc2-c2ccc(-c3ccc(N(c4ccc(-c5ccc6c7ccccc7n(-c7ccccc7)c6c5)cc4)c4cccc(-c5cccc6c5ccc5ccccc56)c4)cc3)cc21 ; CC1(C)c2ccccc2-c2ccc(-c3ccc(N(c4cccc(-c5ccc6c(c5)c5ccccc5n6-c5ccccc5)c4)c4cccc(-c5cccc6c5ccc5ccccc56)c4)cc3)cc21 ; CC1(C)c2ccccc2-c2ccc(-c3ccc(N(c4cccc(-c5cccc6c5ccc5ccccc56)c4)c4cccc(-n5c6ccccc6c6ccccc65)c4)cc3)cc21]

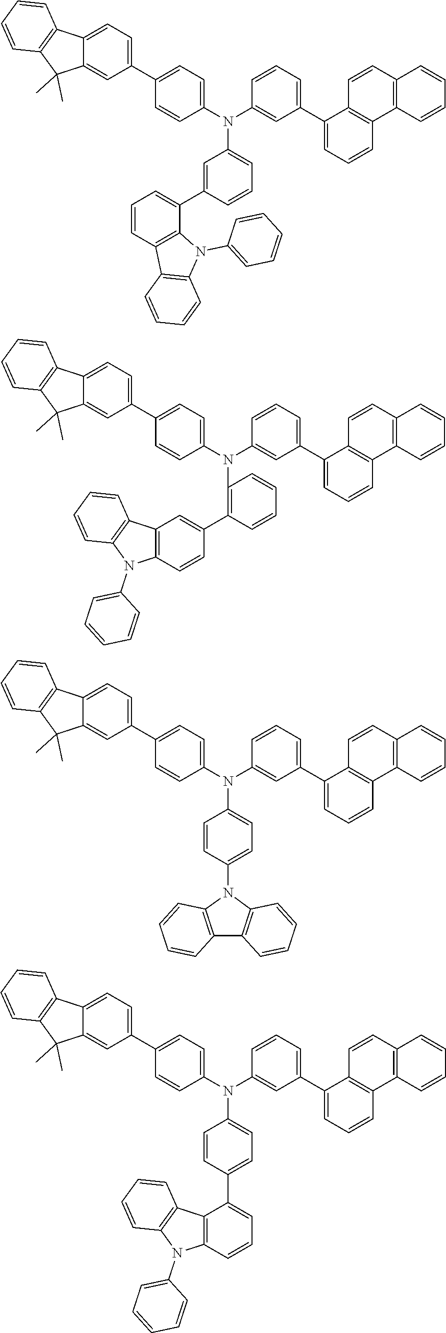

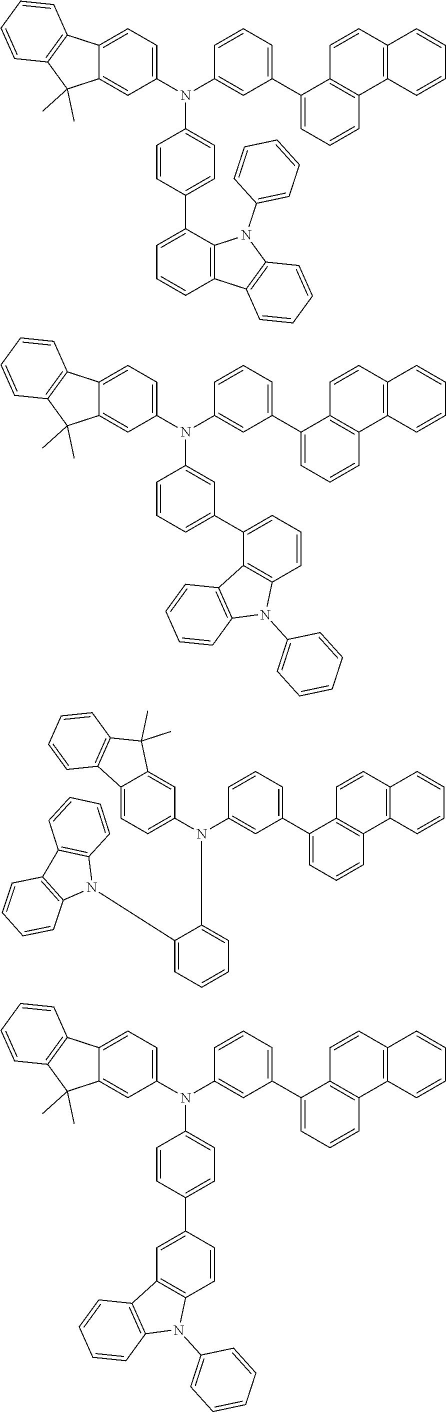 [SMILES: CC1(C)c2ccccc2-c2ccc(N(c3ccc(-c4cccc5c6ccccc6n(-c6ccccc6)c45)cc3)c3cccc(-c4cccc5c4ccc4ccccc45)c3)cc21 ; CC1(C)c2ccccc2-c2ccc(N(c3cccc(-c4cccc5c4ccc4ccccc45)c3)c3cccc(-c4cccc5c4c4ccccc4n5-c4ccccc4)c3)cc21 ; CC1(C)c2ccccc2-c2ccc(N(c3cccc(-c4cccc5c4ccc4ccccc45)c3)c3ccccc3-n3c4ccccc4c4ccccc43)cc21 ; CC1(C)c2ccccc2-c2ccc(N(c3ccc(-c4ccc5c(c4)c4ccccc4n5-c4ccccc4)cc3)c3cccc(-c4cccc5c4ccc4ccccc45)c3)cc21]

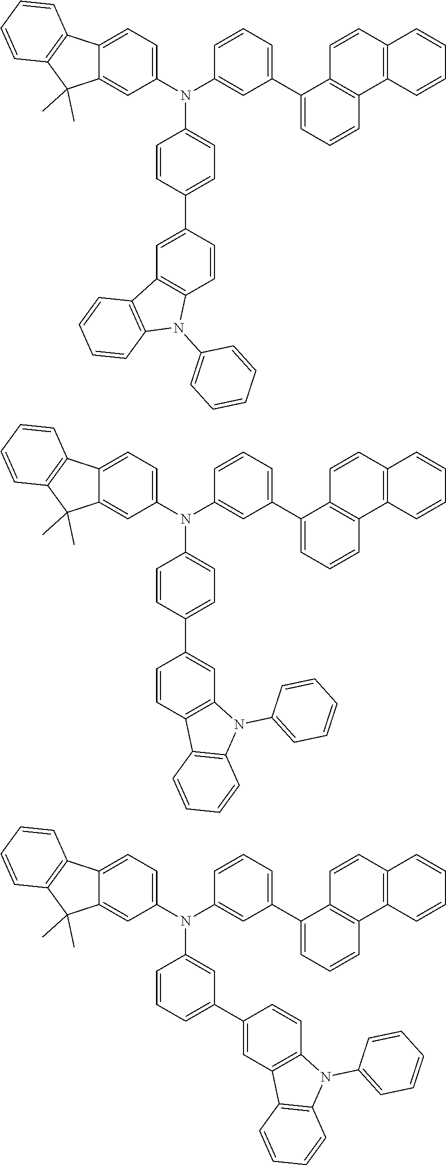 [SMILES: CC1(C)c2ccccc2-c2ccc(N(c3ccc(-c4ccc5c(c4)c4ccccc4n5-c4ccccc4)cc3)c3cccc(-c4cccc5c4ccc4ccccc45)c3)cc21 ; CC1(C)c2ccccc2-c2ccc(N(c3ccc(-c4ccc5c6ccccc6n(-c6ccccc6)c5c4)cc3)c3cccc(-c4cccc5c4ccc4ccccc45)c3)cc21 ; CC1(C)c2ccccc2-c2ccc(N(c3cccc(-c4ccc5c(c4)c4ccccc4n5-c4ccccc4)c3)c3cccc(-c4cccc5c4ccc4ccccc45)c3)cc21]

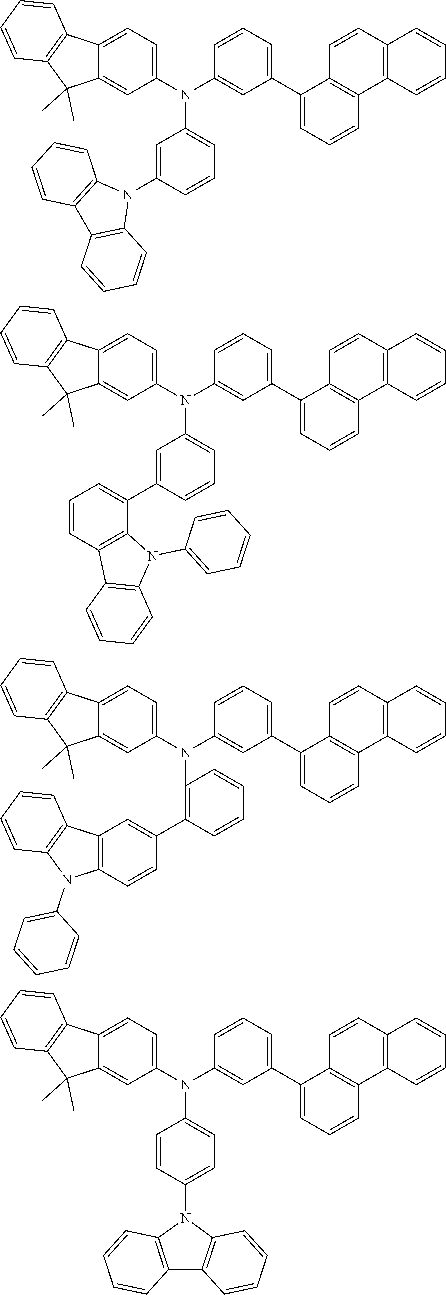

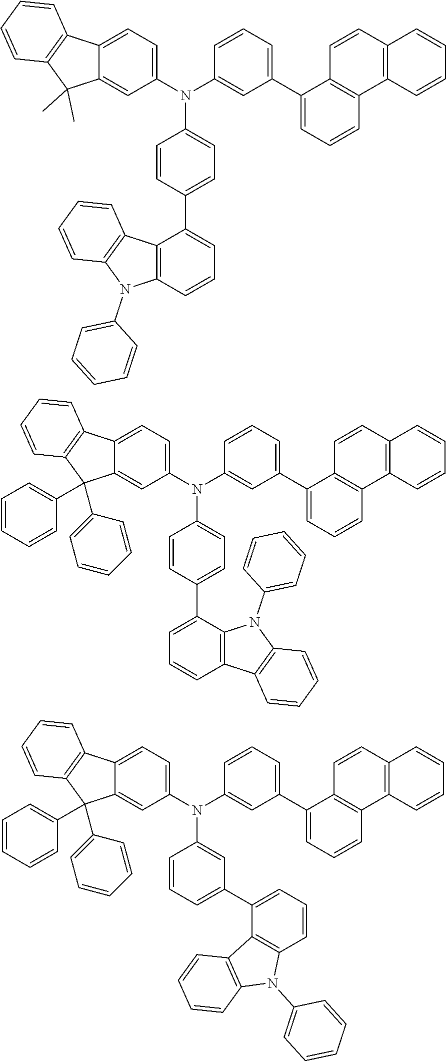

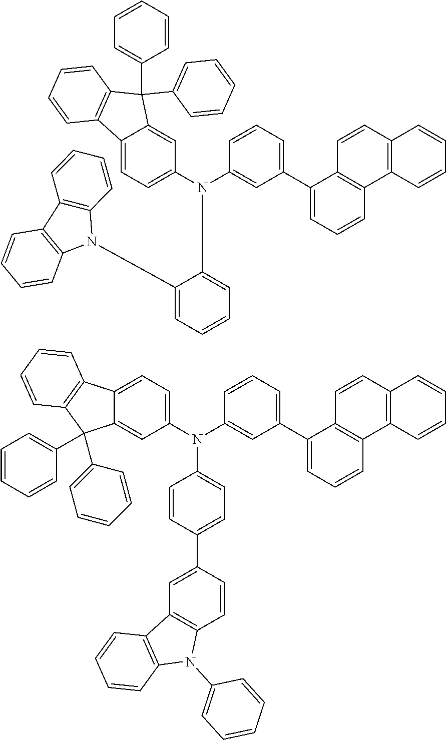 [SMILES: c1ccc(C2(c3ccccc3)c3ccccc3-c3ccc(N(c4cccc(-c5cccc6c5ccc5ccccc56)c4)c4ccccc4-n4c5ccccc5c5ccccc54)cc32)cc1 ; c1ccc(-n2c3ccccc3c3cc(-c4ccc(N(c5cccc(-c6cccc7c6ccc6ccccc67)c5)c5ccc6c(c5)C(c5ccccc5)(c5ccccc5)c5ccccc5-6)cc4)ccc32)cc1]

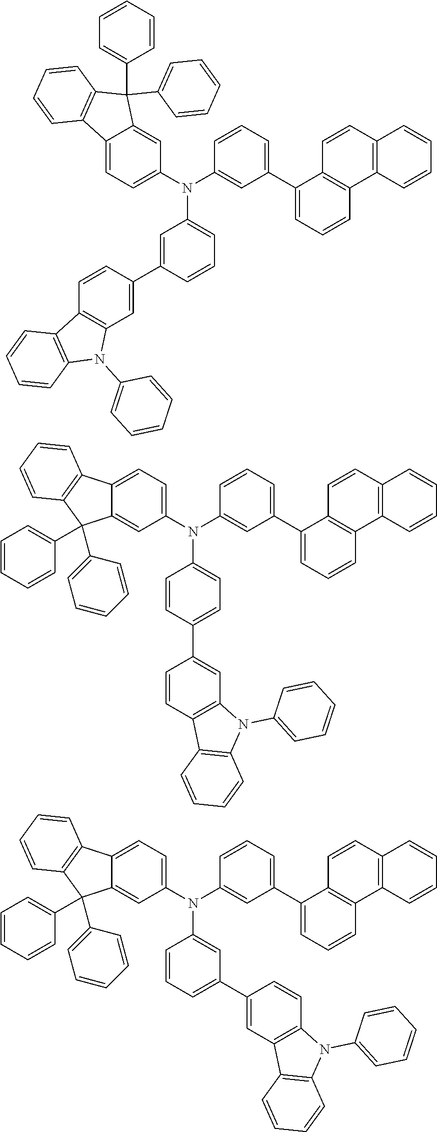

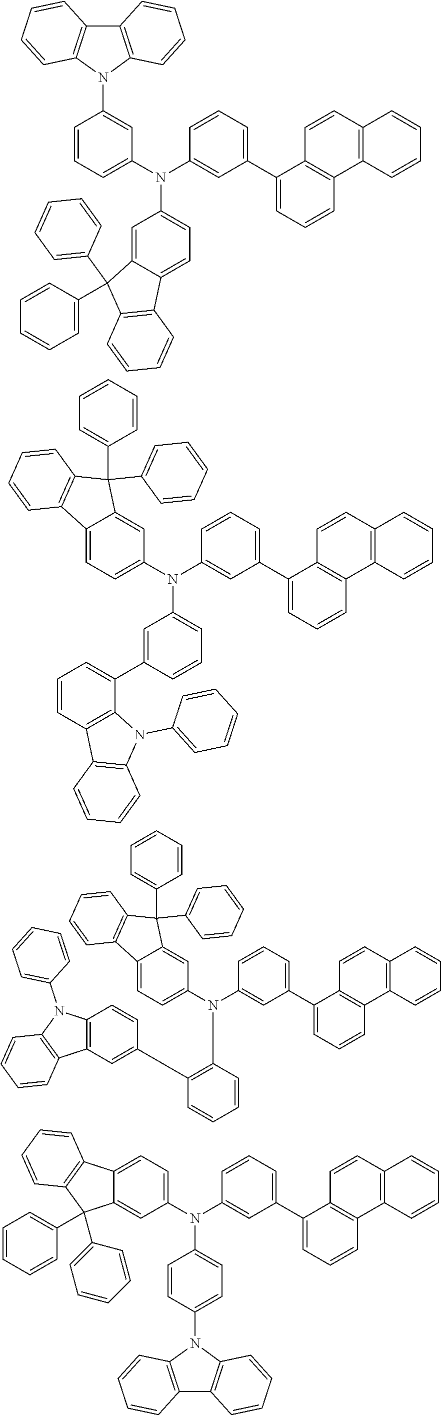

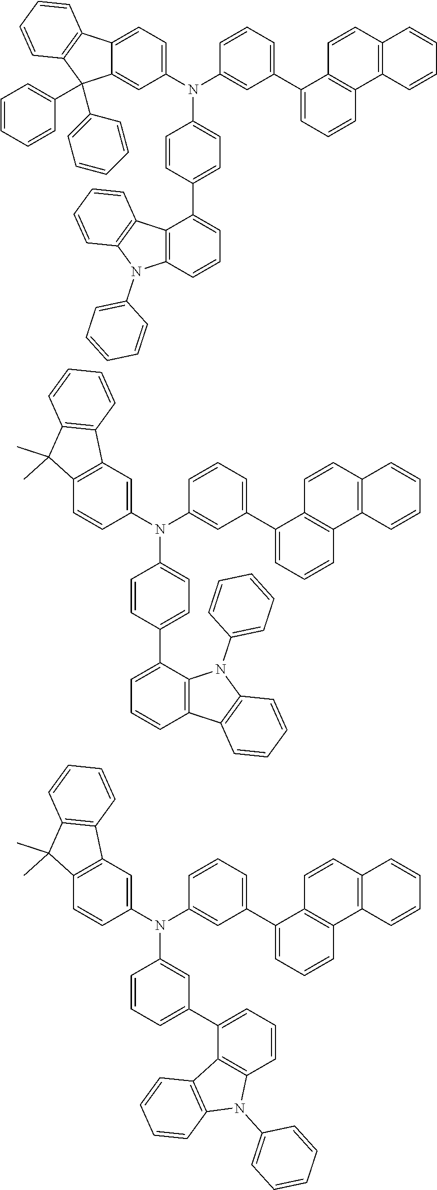

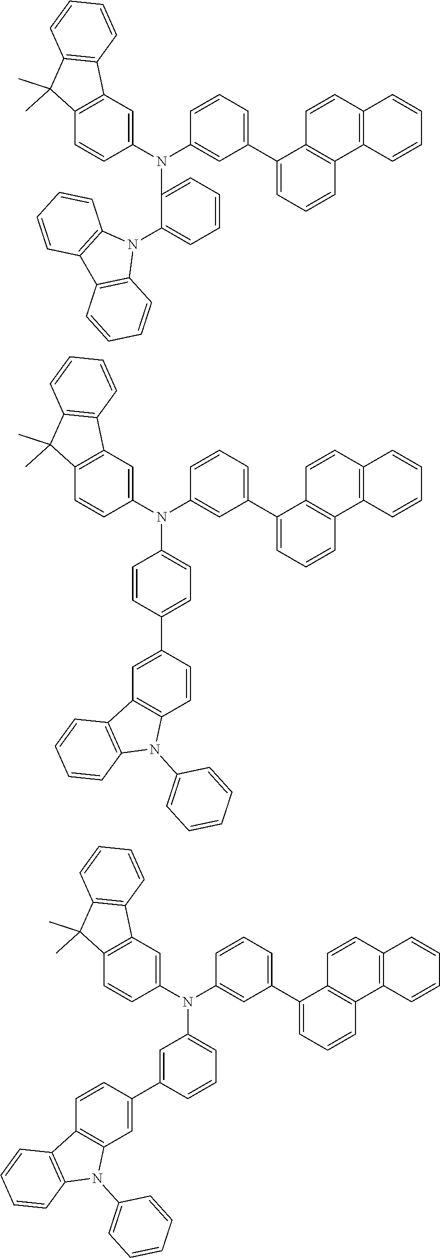

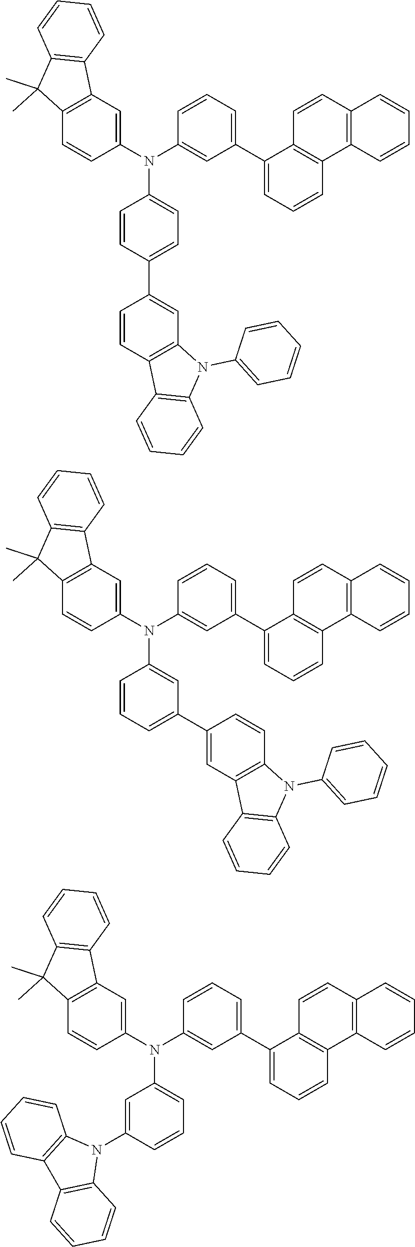 [SMILES: CC1(C)c2ccccc2-c2cc(N(c3ccc(-c4ccc5c6ccccc6n(-c6ccccc6)c5c4)cc3)c3cccc(-c4cccc5c4ccc4ccccc45)c3)ccc21 ; CC1(C)c2ccccc2-c2cc(N(c3cccc(-c4ccc5c(c4)c4ccccc4n5-c4ccccc4)c3)c3cccc(-c4cccc5c4ccc4ccccc45)c3)ccc21 ; CC1(C)c2ccccc2-c2cc(N(c3cccc(-c4cccc5c4ccc4ccccc45)c3)c3cccc(-n4c5ccccc5c5ccccc54)c3)ccc21]

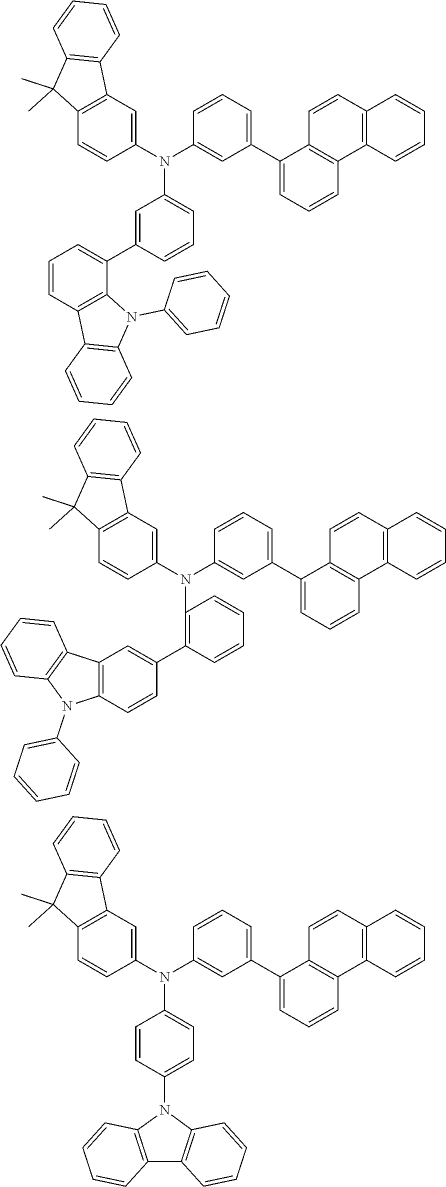

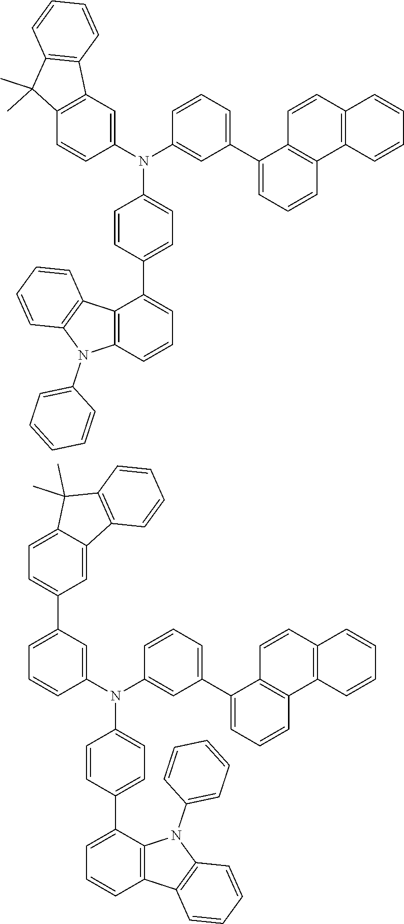

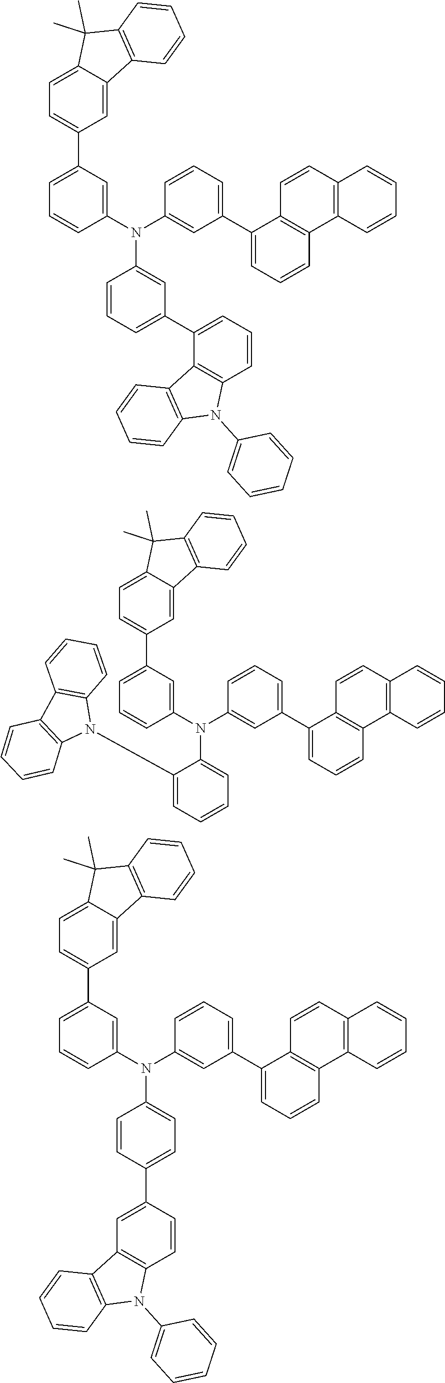

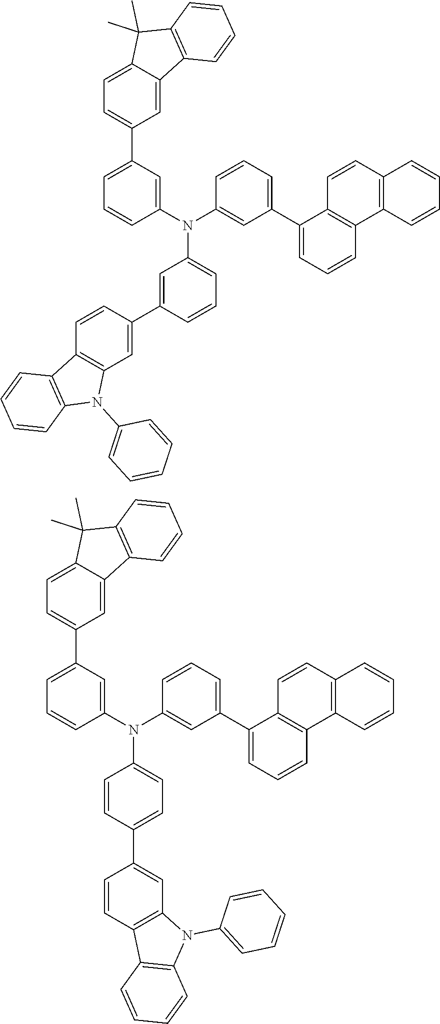

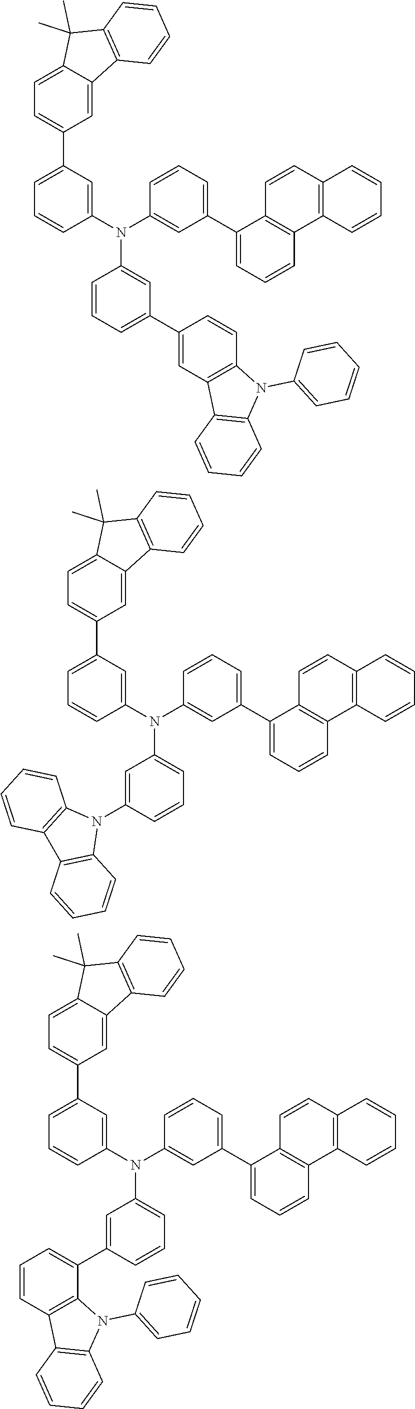

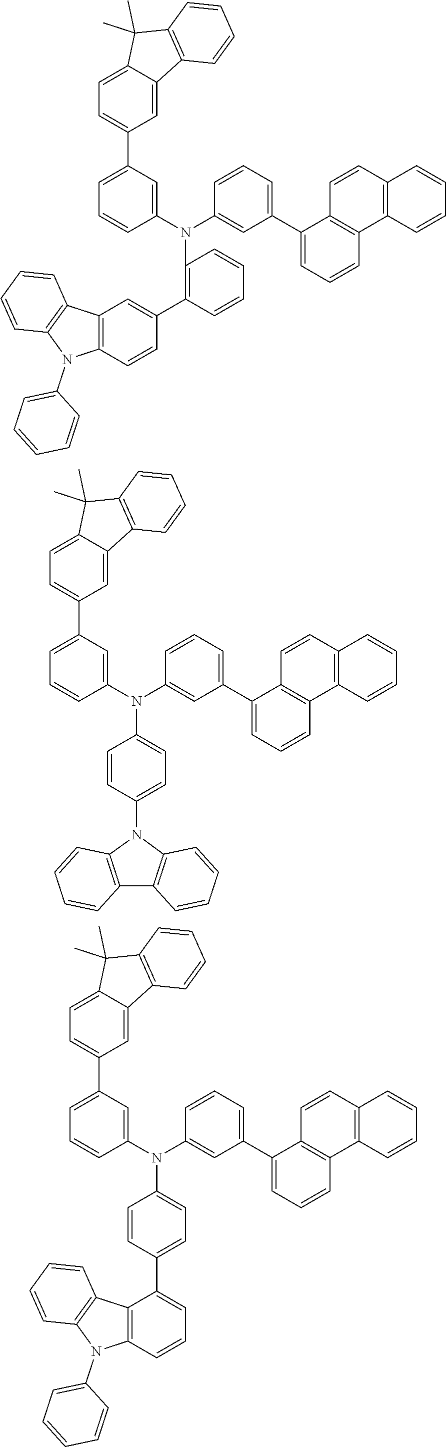

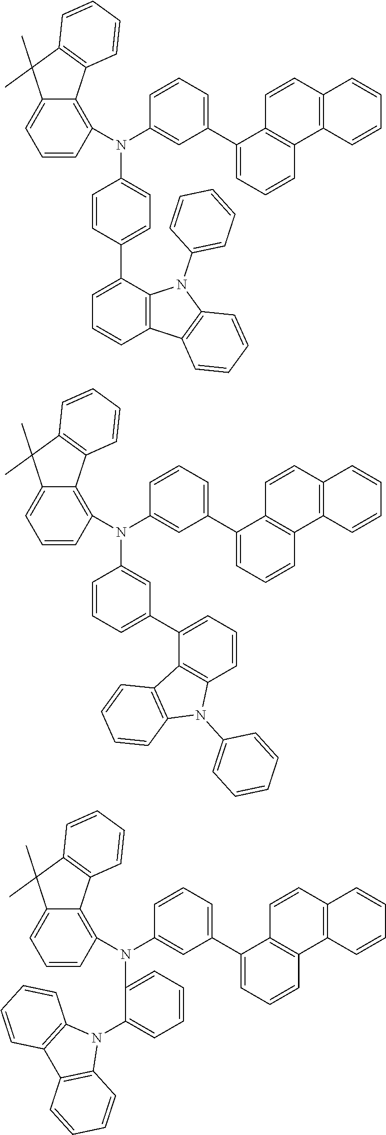 [SMILES: CC1(C)c2ccccc2-c2c(N(c3ccc(-c4cccc5c6ccccc6n(-c6ccccc6)c45)cc3)c3cccc(-c4cccc5c4ccc4ccccc45)c3)cccc21 ; CC1(C)c2ccccc2-c2c(N(c3cccc(-c4cccc5c4ccc4ccccc45)c3)c3cccc(-c4cccc5c4c4ccccc4n5-c4ccccc4)c3)cccc21 ; CC1(C)c2ccccc2-c2c(N(c3cccc(-c4cccc5c4ccc4ccccc45)c3)c3ccccc3-n3c4ccccc4c4ccccc43)cccc21]

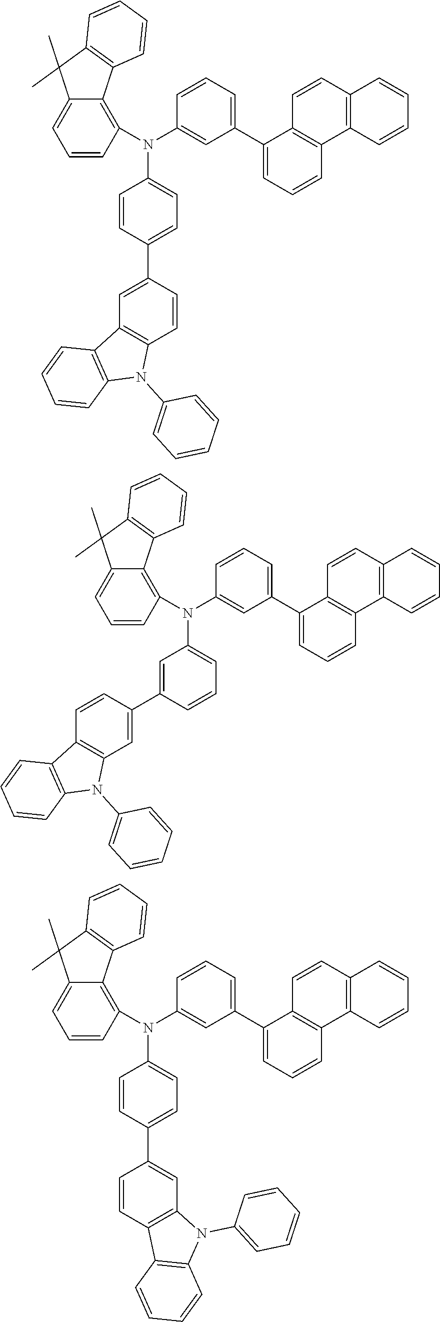

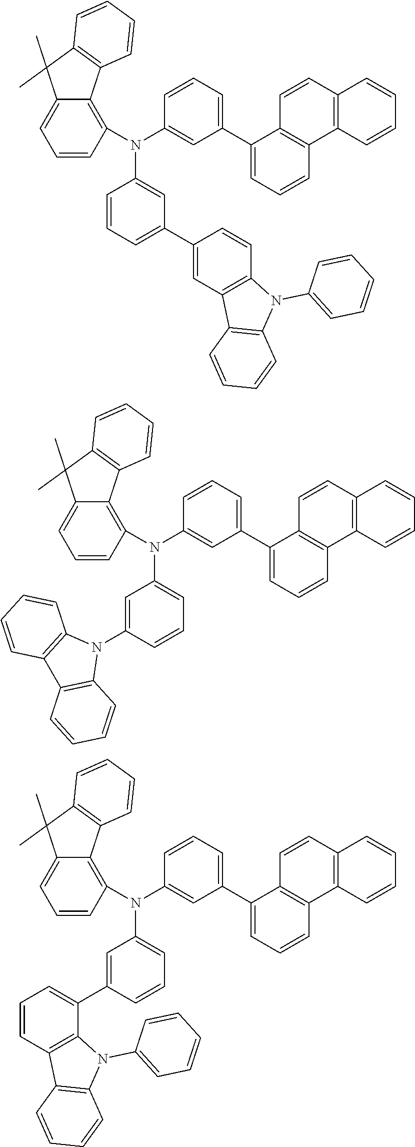

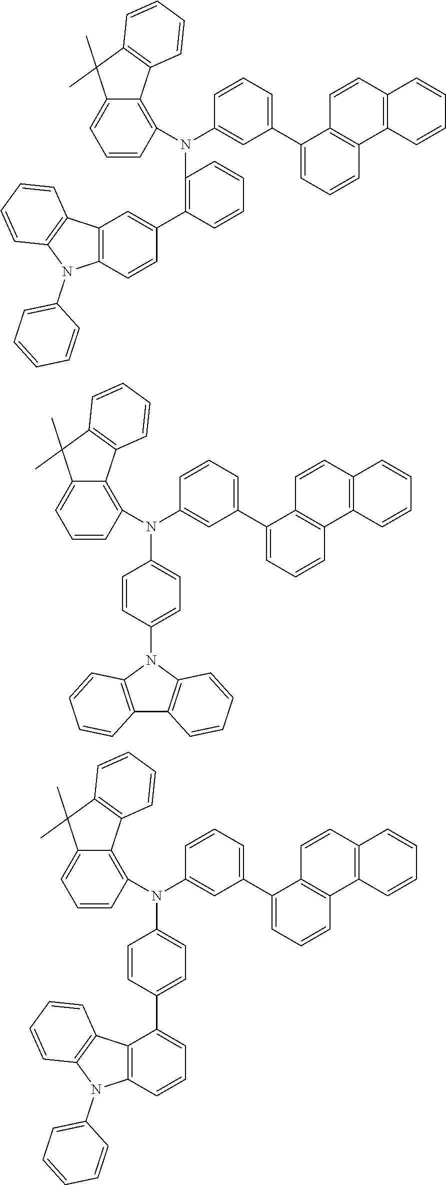

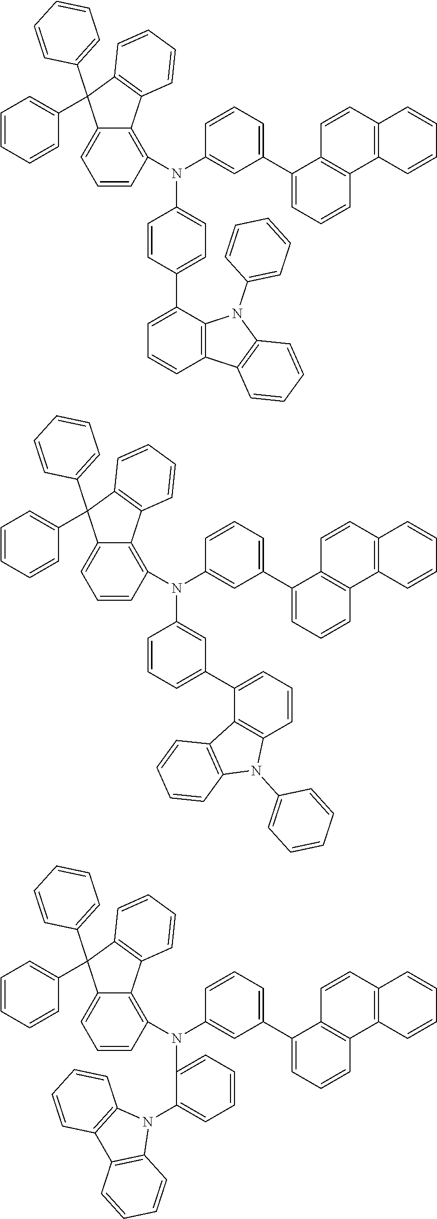 [SMILES: c1ccc(-n2c3ccccc3c3cccc(-c4ccc(N(c5cccc(-c6cccc7c6ccc6ccccc67)c5)c5cccc6c5-c5ccccc5C6(c5ccccc5)c5ccccc5)cc4)c32)cc1 ; c1ccc(-n2c3ccccc3c3c(-c4cccc(N(c5cccc(-c6cccc7c6ccc6ccccc67)c5)c5cccc6c5-c5ccccc5C6(c5ccccc5)c5ccccc5)c4)cccc32)cc1 ; c1ccc(C2(c3ccccc3)c3ccccc3-c3c(N(c4cccc(-c5cccc6c5ccc5ccccc56)c4)c4ccccc4-n4c5ccccc5c5ccccc54)cccc32)cc1]

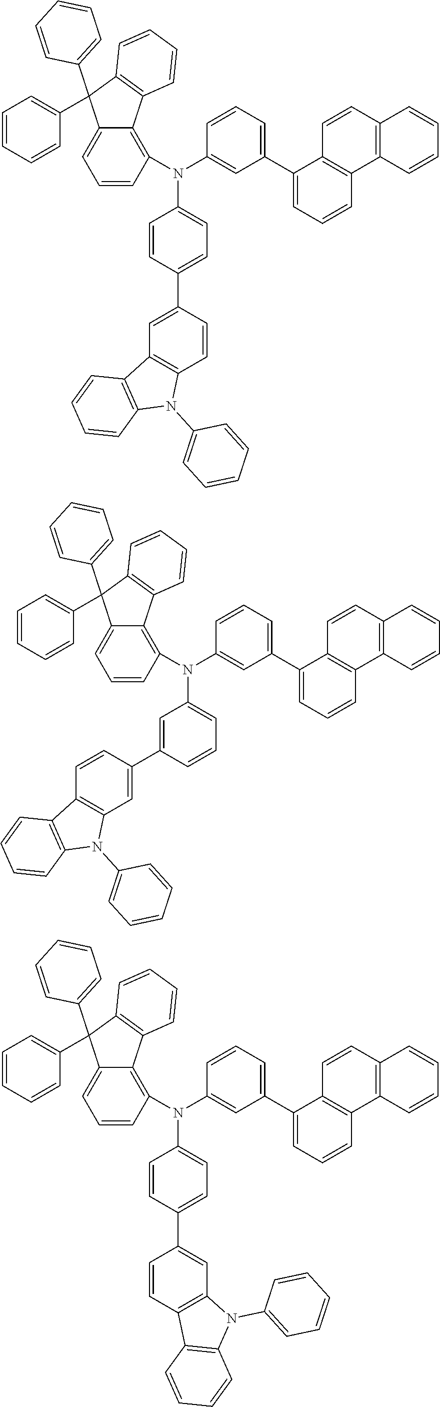

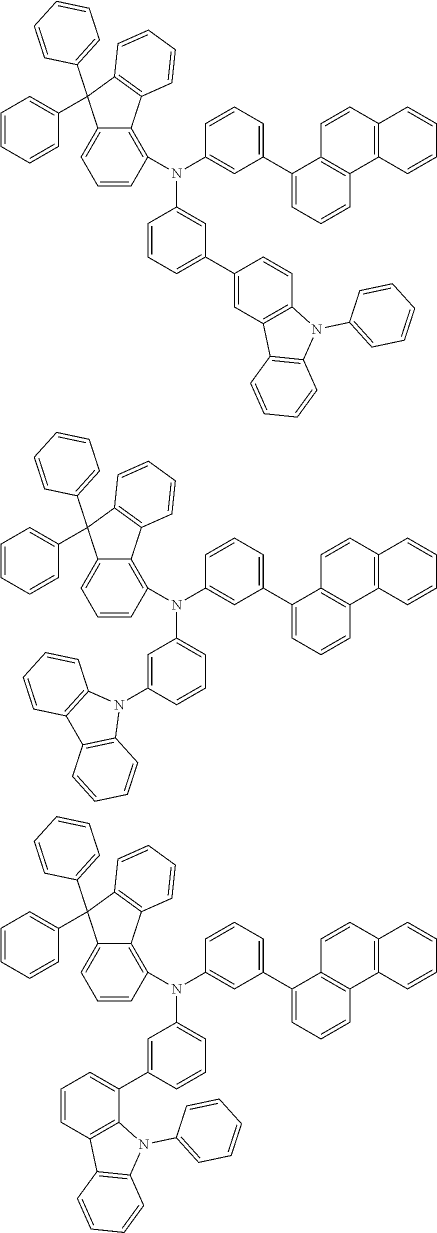

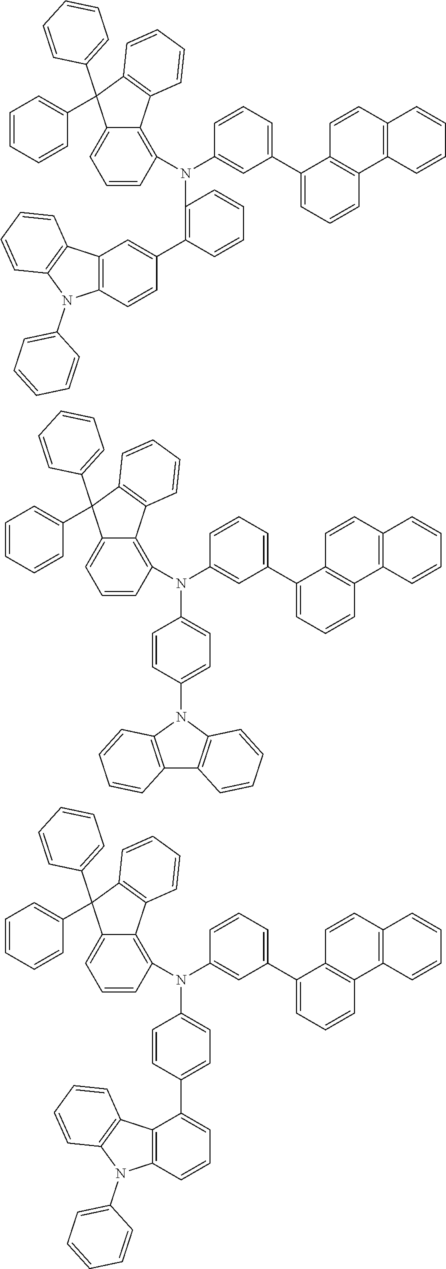

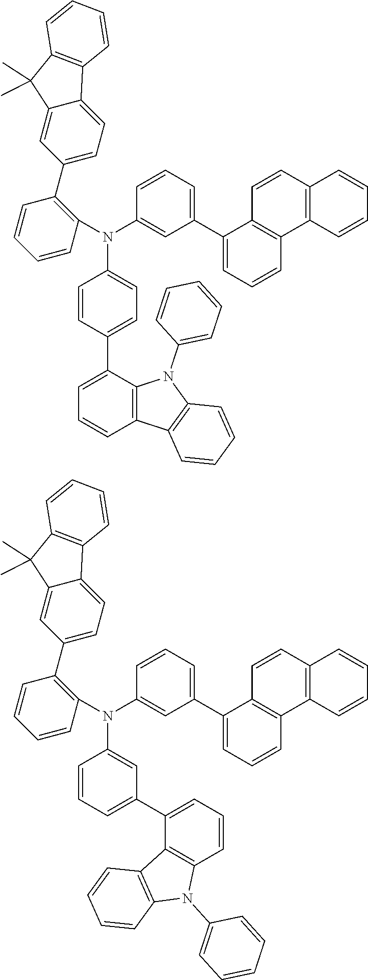 [SMILES: CC1(C)c2ccccc2-c2ccc(-c3ccccc3N(c3ccc(-c4cccc5c6ccccc6n(-c6ccccc6)c45)cc3)c3cccc(-c4cccc5c4ccc4ccccc45)c3)cc21 ; CC1(C)c2ccccc2-c2ccc(-c3ccccc3N(c3cccc(-c4cccc5c4ccc4ccccc45)c3)c3cccc(-c4cccc5c4c4ccccc4n5-c4ccccc4)c3)cc21]

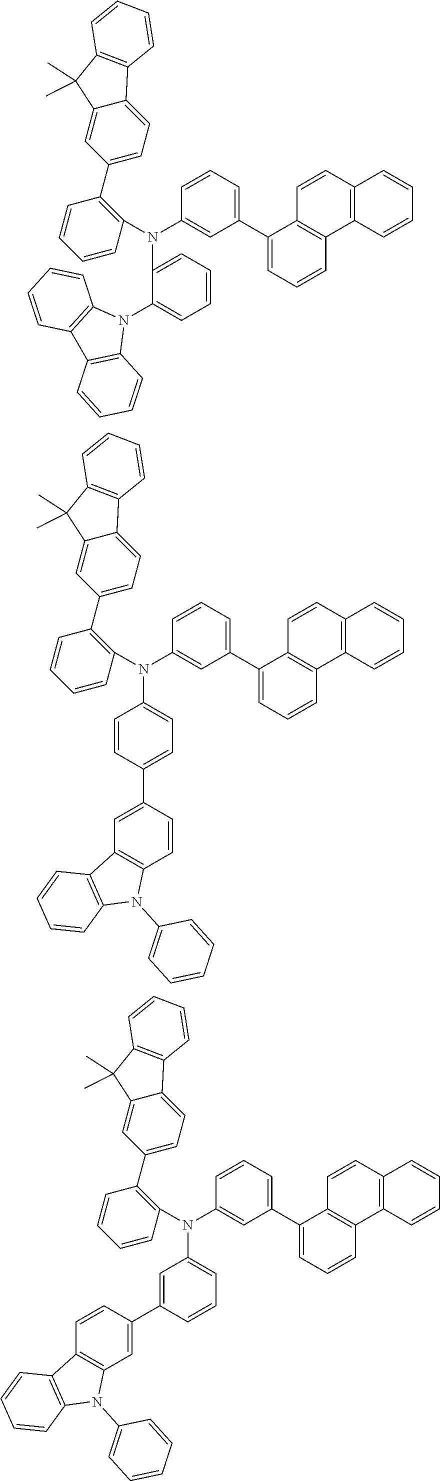

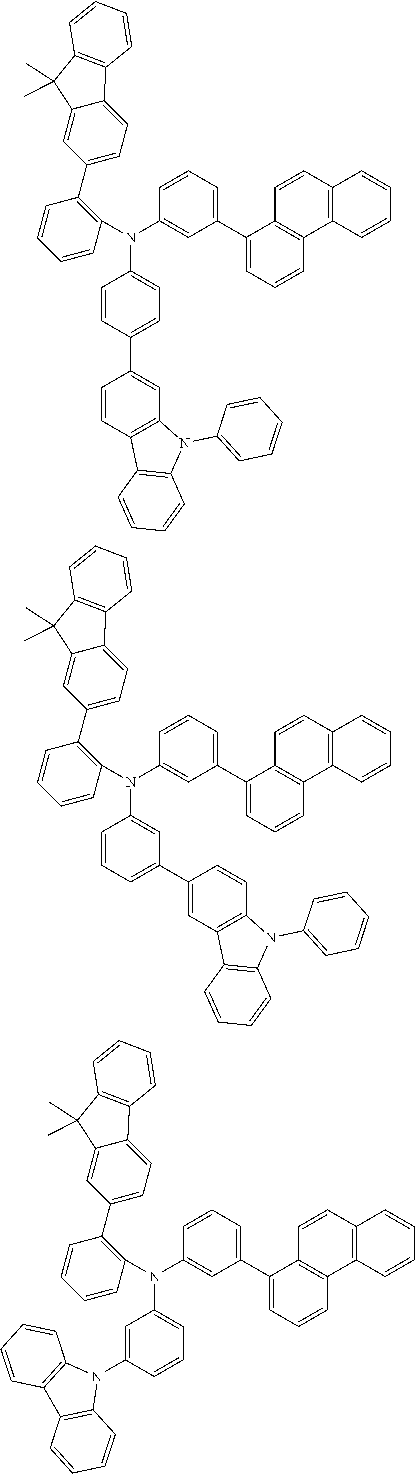

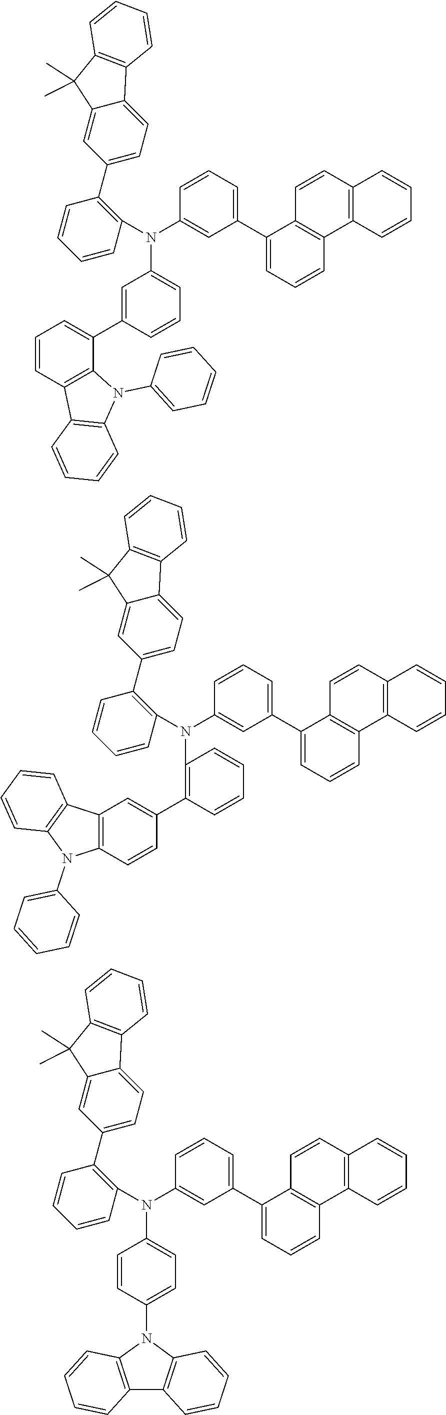

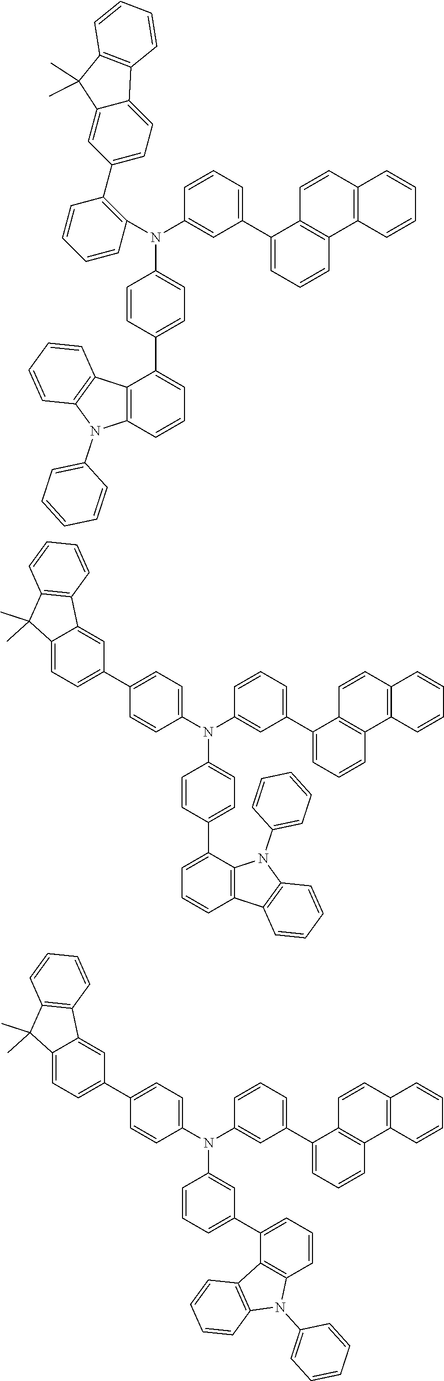

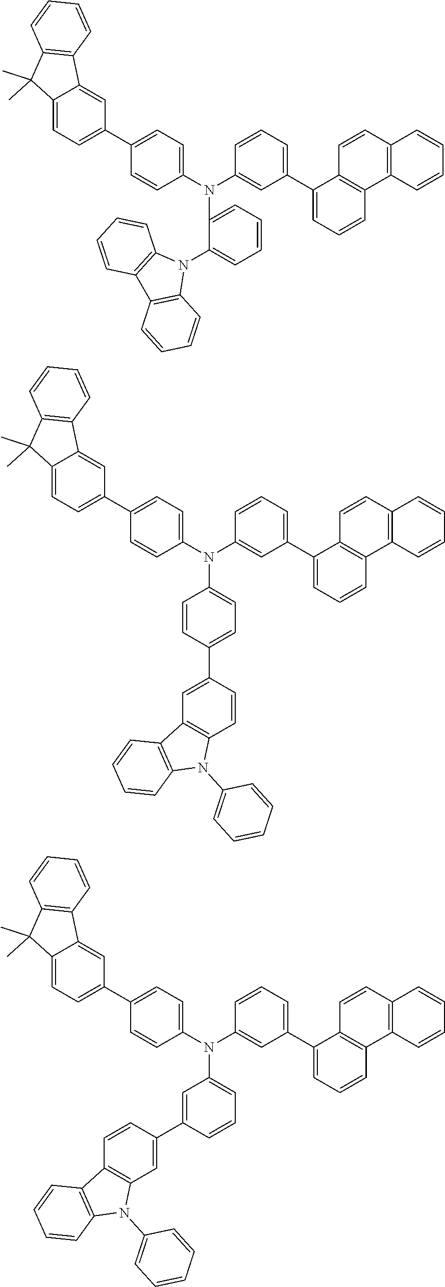 [SMILES: CC1(C)c2ccccc2-c2cc(-c3ccc(N(c4cccc(-c5cccc6c5ccc5ccccc56)c4)c4ccccc4-n4c5ccccc5c5ccccc54)cc3)ccc21 ; CC1(C)c2ccccc2-c2cc(-c3ccc(N(c4ccc(-c5ccc6c(c5)c5ccccc5n6-c5ccccc5)cc4)c4cccc(-c5cccc6c5ccc5ccccc56)c4)cc3)ccc21 ; CC1(C)c2ccccc2-c2cc(-c3ccc(N(c4cccc(-c5ccc6c7ccccc7n(-c7ccccc7)c6c5)c4)c4cccc(-c5cccc6c5ccc5ccccc56)c4)cc3)ccc21]

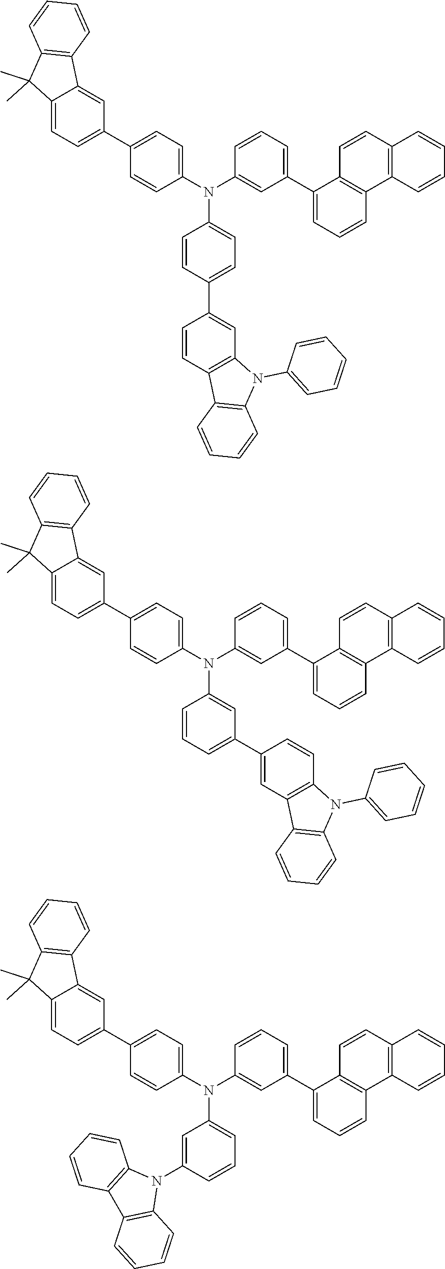 [SMILES: CC1(C)c2ccccc2-c2cc(-c3ccc(N(c4ccc(-c5ccc6c7ccccc7n(-c7ccccc7)c6c5)cc4)c4cccc(-c5cccc6c5ccc5ccccc56)c4)cc3)ccc21 ; CC1(C)c2ccccc2-c2cc(-c3ccc(N(c4cccc(-c5ccc6c(c5)c5ccccc5n6-c5ccccc5)c4)c4cccc(-c5cccc6c5ccc5ccccc56)c4)cc3)ccc21 ; CC1(C)c2ccccc2-c2cc(-c3ccc(N(c4cccc(-c5cccc6c5ccc5ccccc56)c4)c4cccc(-n5c6ccccc6c6ccccc65)c4)cc3)ccc21]

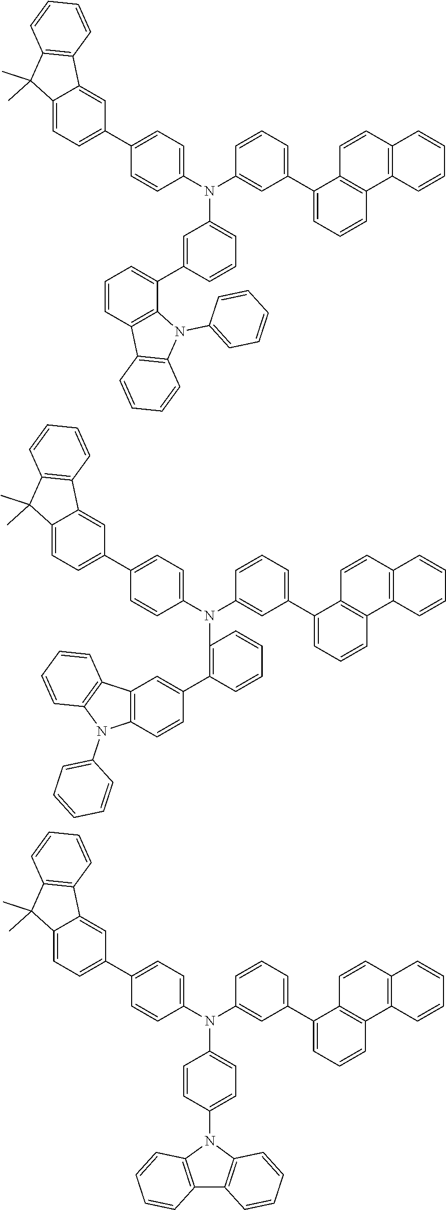

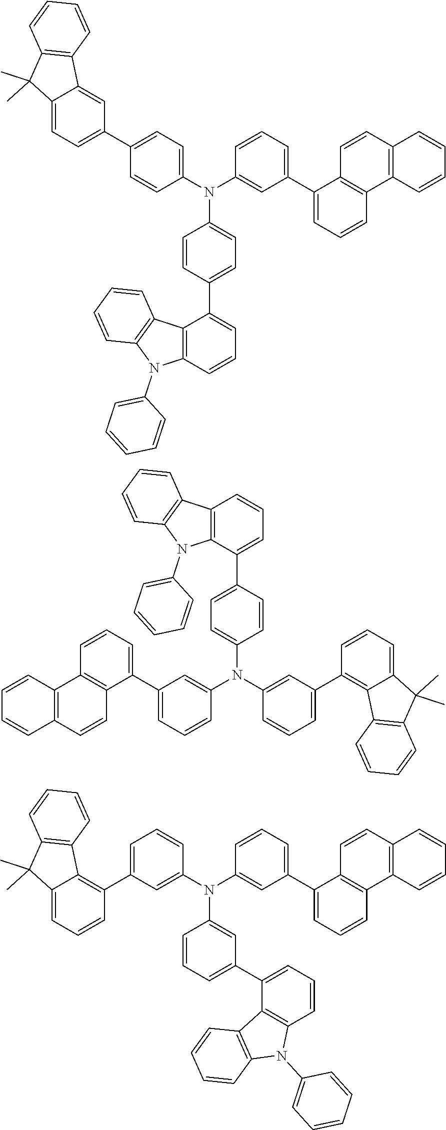

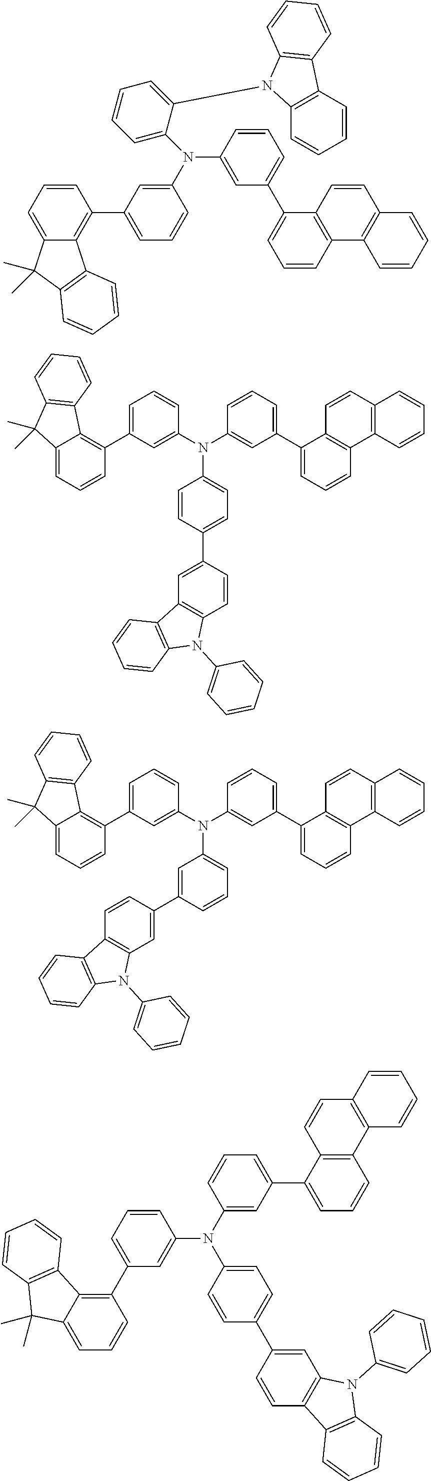

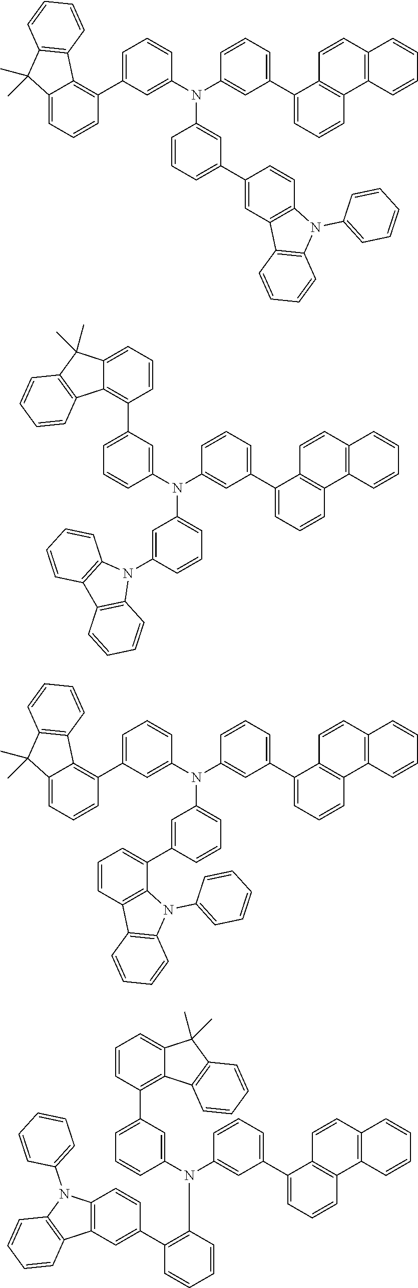 [SMILES: CC1(C)c2ccccc2-c2c(-c3cccc(N(c4cccc(-c5ccc6c(c5)c5ccccc5n6-c5ccccc5)c4)c4cccc(-c5cccc6c5ccc5ccccc56)c4)c3)cccc21 ; CC1(C)c2ccccc2-c2c(-c3cccc(N(c4cccc(-c5cccc6c5ccc5ccccc56)c4)c4cccc(-n5c6ccccc6c6ccccc65)c4)c3)cccc21 ; CC1(C)c2ccccc2-c2c(-c3cccc(N(c4cccc(-c5cccc6c5ccc5ccccc56)c4)c4cccc(-c5cccc6c7ccccc7n(-c7ccccc7)c56)c4)c3)cccc21 ; CC1(C)c2ccccc2-c2c(-c3cccc(N(c4cccc(-c5cccc6c5ccc5ccccc56)c4)c4ccccc4-c4ccc5c(c4)c4ccccc4n5-c4ccccc4)c3)cccc21]

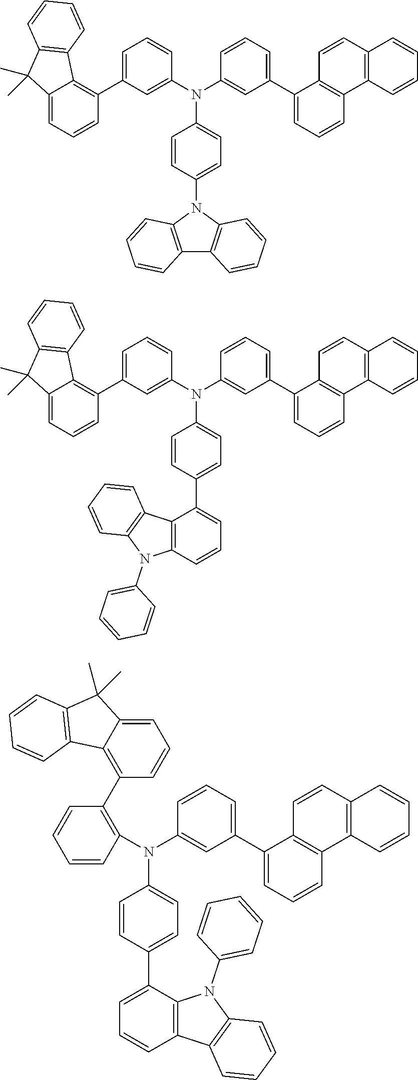

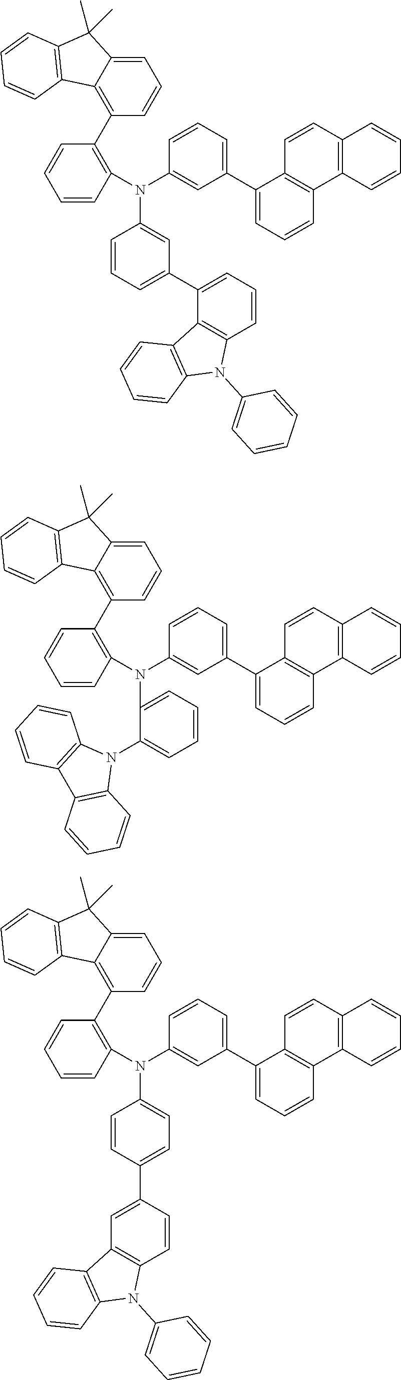

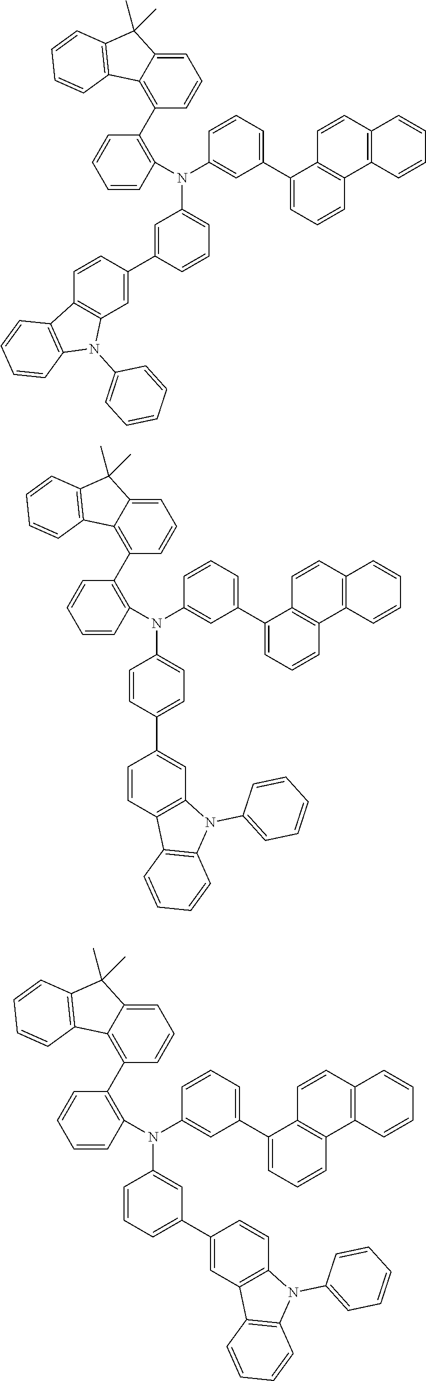

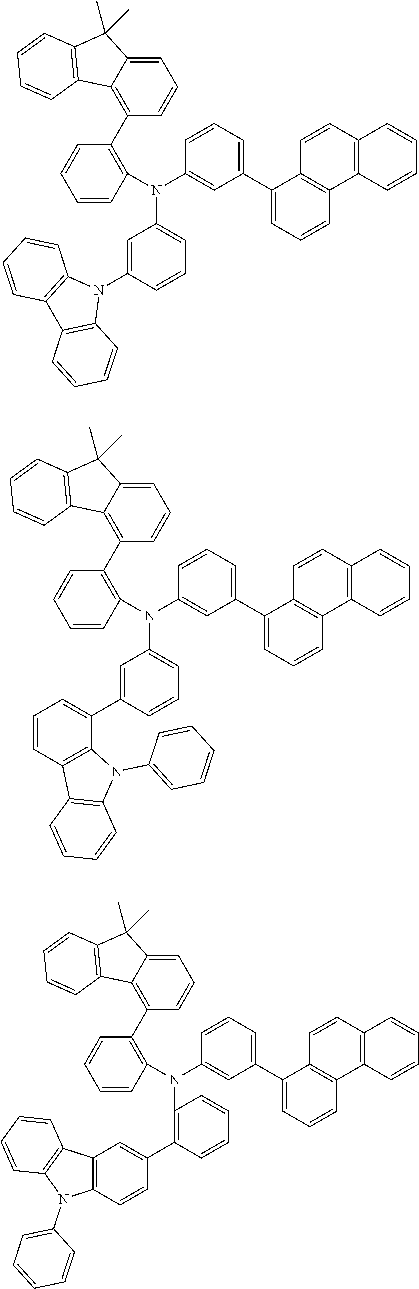

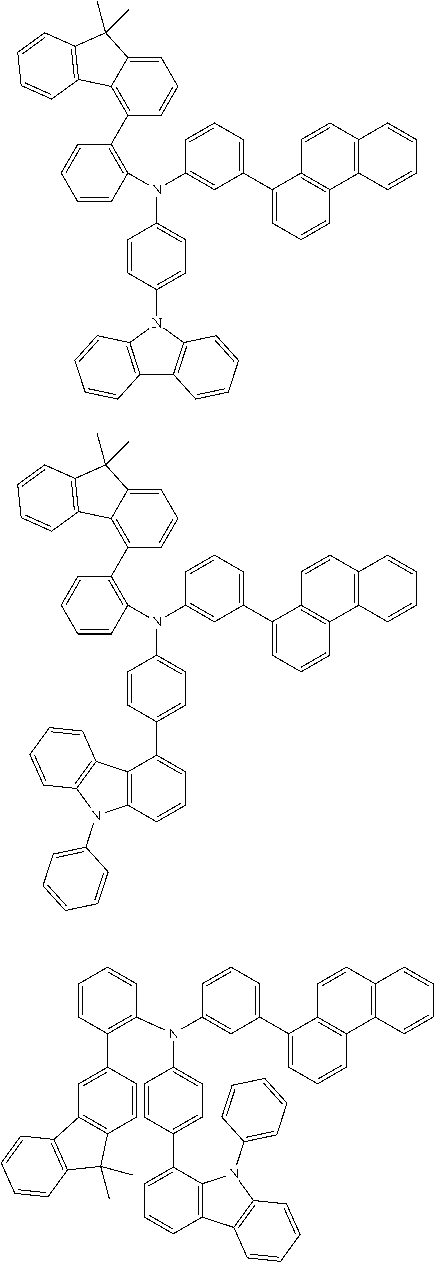

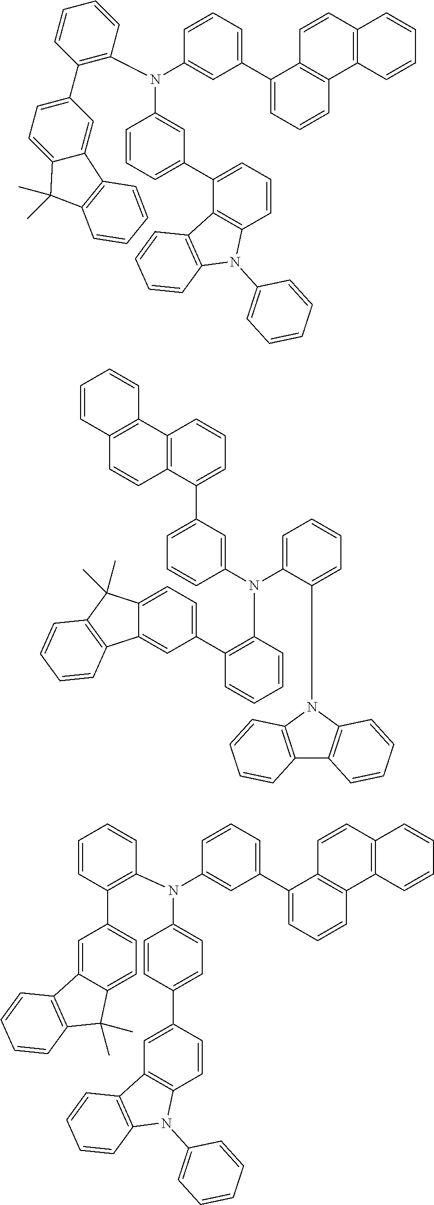

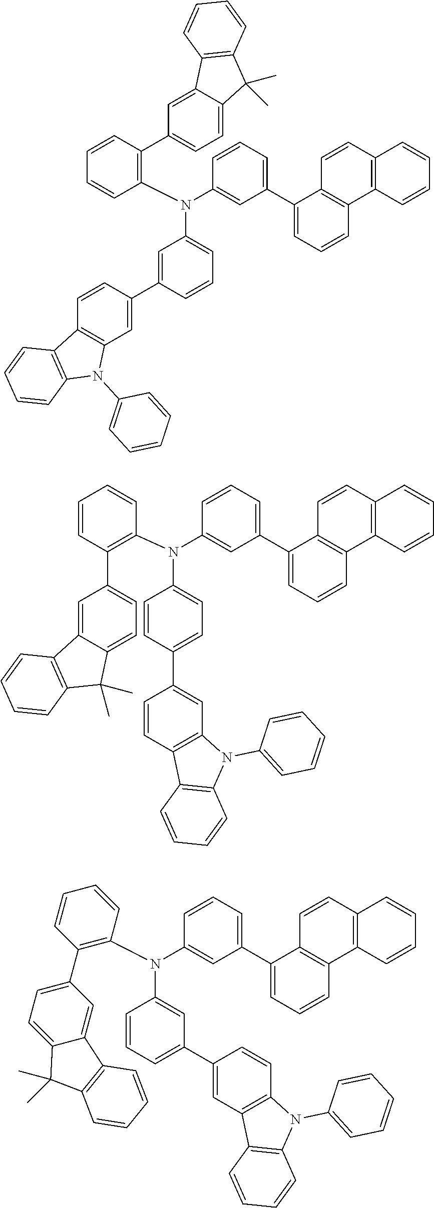

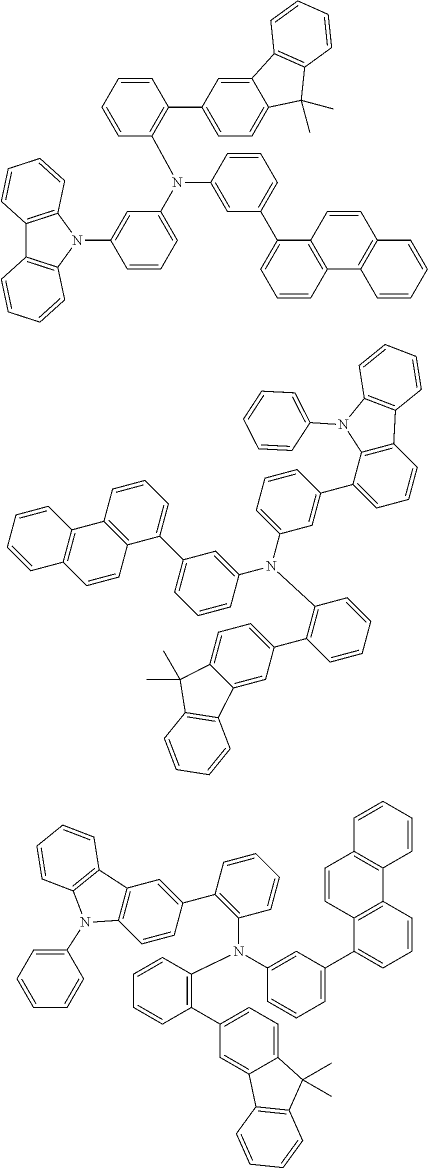 [SMILES: CC1(C)c2ccccc2-c2cc(-c3ccccc3N(c3cccc(-c4cccc5c4ccc4ccccc45)c3)c3cccc(-n4c5ccccc5c5ccccc54)c3)ccc21 ; CC1(C)c2ccccc2-c2cc(-c3ccccc3N(c3cccc(-c4cccc5c4ccc4ccccc45)c3)c3cccc(-c4cccc5c6ccccc6n(-c6ccccc6)c45)c3)ccc21 ; CC1(C)c2ccccc2-c2cc(-c3ccccc3N(c3cccc(-c4cccc5c4ccc4ccccc45)c3)c3ccccc3-c3ccc4c(c3)c3ccccc3n4-c3ccccc3)ccc21]

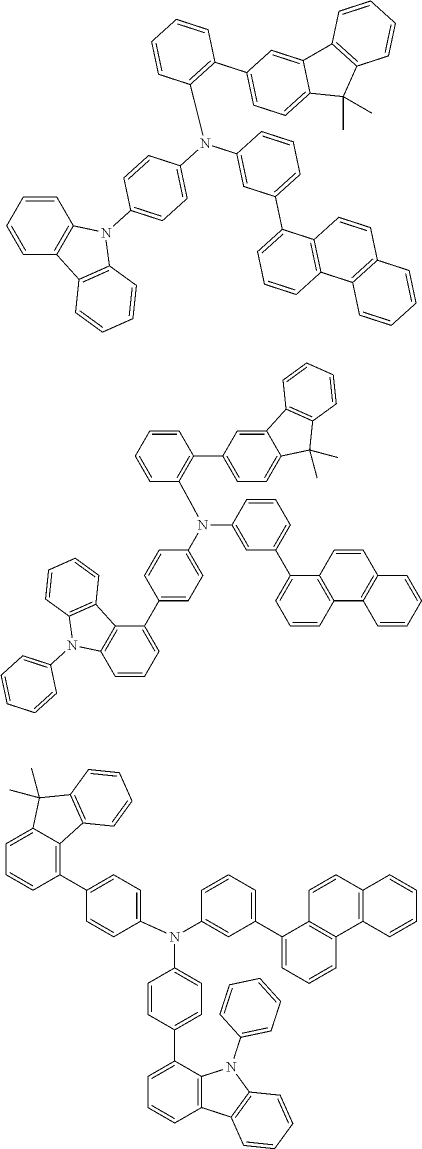 [SMILES: CC1(C)c2ccccc2-c2cc(-c3ccccc3N(c3ccc(-n4c5ccccc5c5ccccc54)cc3)c3cccc(-c4cccc5c4ccc4ccccc45)c3)ccc21 ; CC1(C)c2ccccc2-c2cc(-c3ccccc3N(c3ccc(-c4cccc5c4c4ccccc4n5-c4ccccc4)cc3)c3cccc(-c4cccc5c4ccc4ccccc45)c3)ccc21 ; CC1(C)c2ccccc2-c2c(-c3ccc(N(c4ccc(-c5cccc6c7ccccc7n(-c7ccccc7)c56)cc4)c4cccc(-c5cccc6c5ccc5ccccc56)c4)cc3)cccc21]

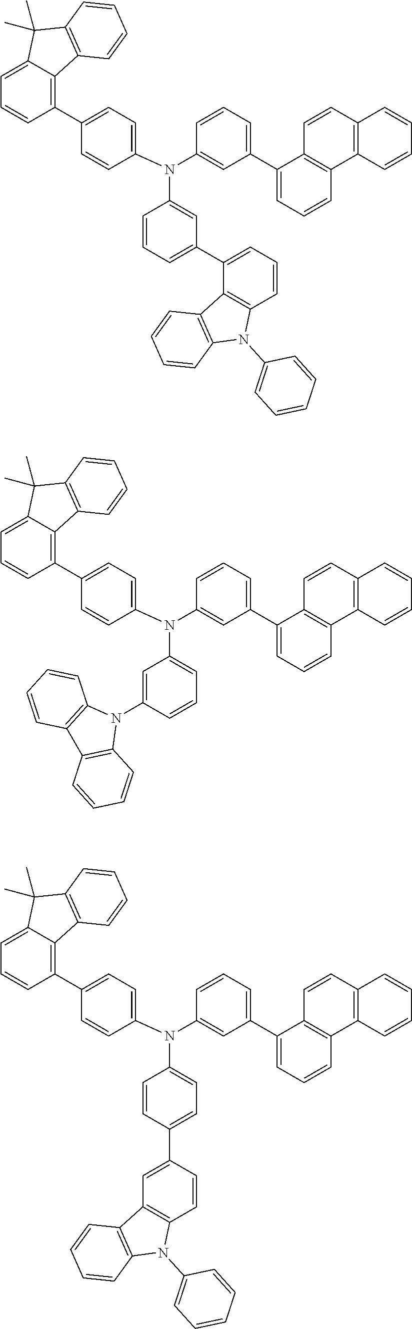 [SMILES: CC1(C)c2ccccc2-c2c(-c3ccc(N(c4cccc(-c5cccc6c5ccc5ccccc56)c4)c4cccc(-c5cccc6c5c5ccccc5n6-c5ccccc5)c4)cc3)cccc21 ; CC1(C)c2ccccc2-c2c(-c3ccc(N(c4cccc(-c5cccc6c5ccc5ccccc56)c4)c4cccc(-n5c6ccccc6c6ccccc65)c4)cc3)cccc21 ; CC1(C)c2ccccc2-c2c(-c3ccc(N(c4ccc(-c5ccc6c(c5)c5ccccc5n6-c5ccccc5)cc4)c4cccc(-c5cccc6c5ccc5ccccc56)c4)cc3)cccc21]

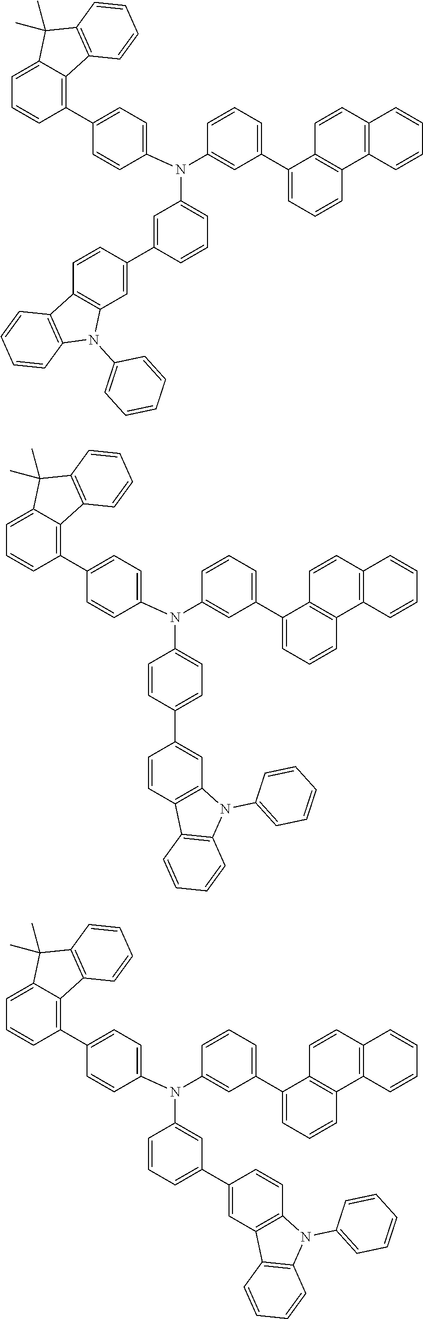

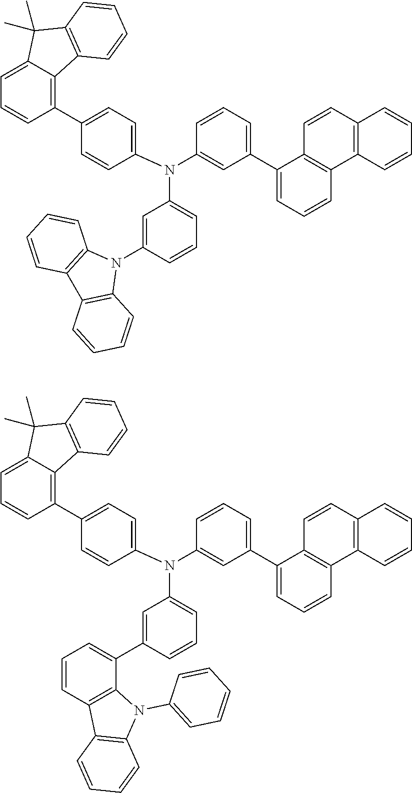 [SMILES: CC1(C)c2ccccc2-c2c(-c3ccc(N(c4cccc(-c5cccc6c5ccc5ccccc56)c4)c4cccc(-n5c6ccccc6c6ccccc65)c4)cc3)cccc21 ; CC1(C)c2ccccc2-c2c(-c3ccc(N(c4cccc(-c5cccc6c5ccc5ccccc56)c4)c4cccc(-c5cccc6c7ccccc7n(-c7ccccc7)c56)c4)cc3)cccc21]

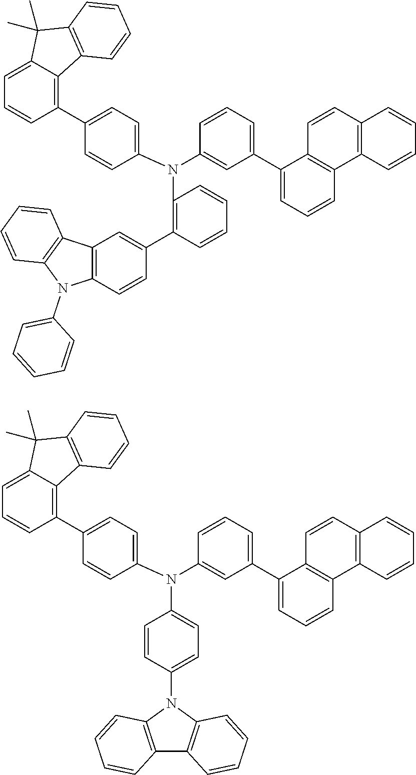

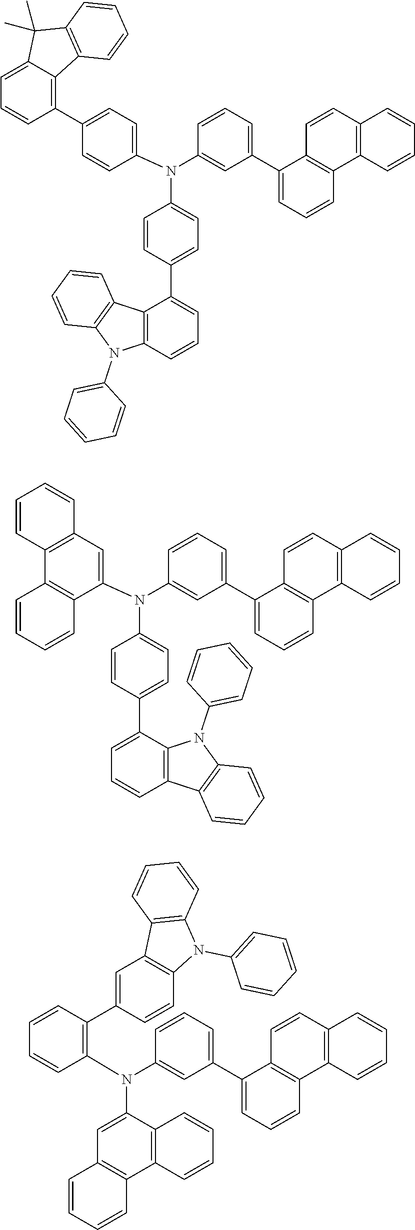 [SMILES: CC1(C)c2ccccc2-c2c(-c3ccc(N(c4ccc(-c5cccc6c5c5ccccc5n6-c5ccccc5)cc4)c4cccc(-c5cccc6c5ccc5ccccc56)c4)cc3)cccc21 ; c1ccc(-n2c3ccccc3c3cccc(-c4ccc(N(c5cccc(-c6cccc7c6ccc6ccccc67)c5)c5cc6ccccc6c6ccccc56)cc4)c32)cc1 ; c1ccc(-n2c3ccccc3c3cc(-c4ccccc4N(c4cccc(-c5cccc6c5ccc5ccccc56)c4)c4cc5ccccc5c5ccccc45)ccc32)cc1]

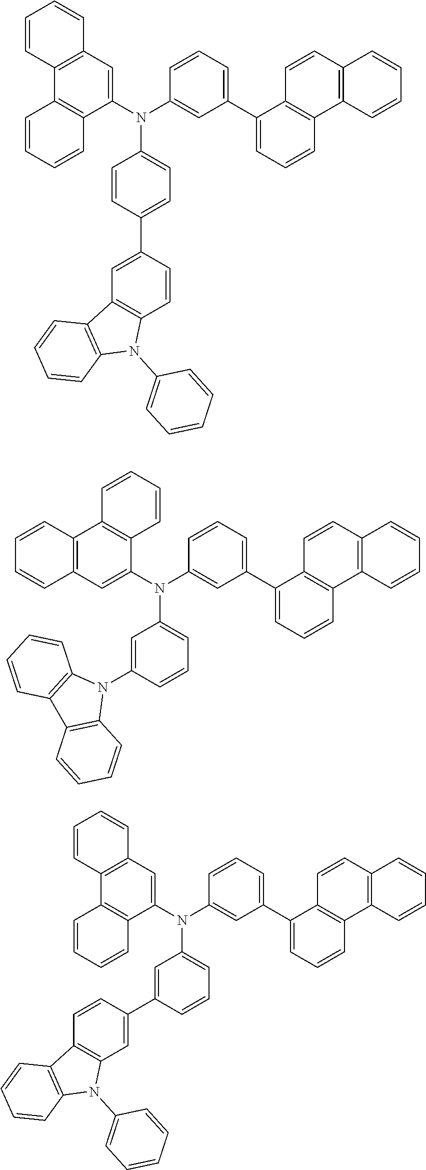

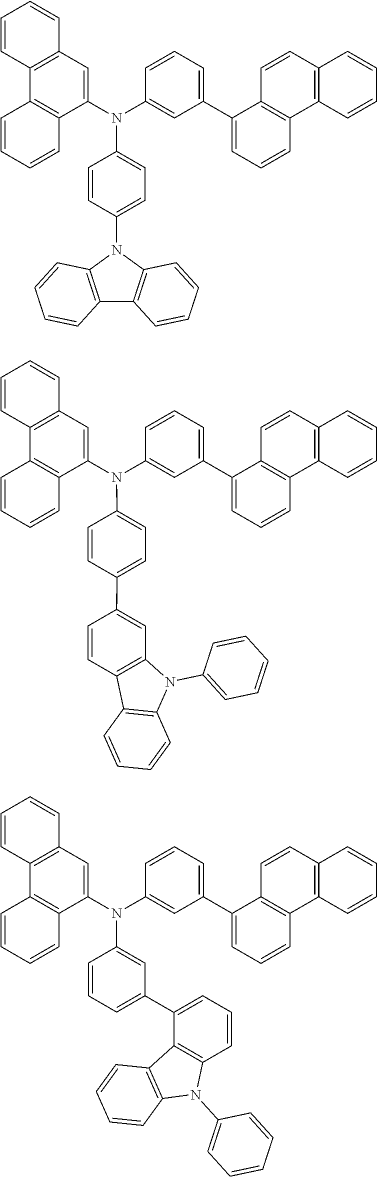

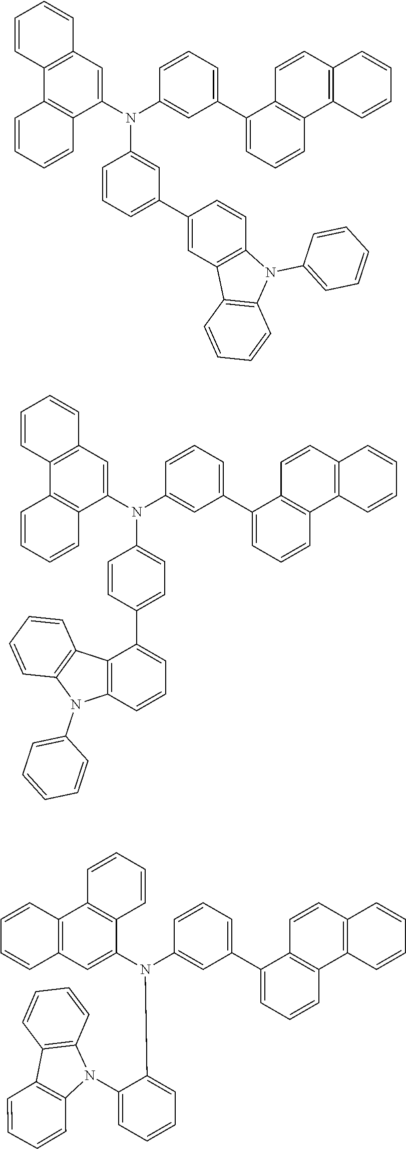 [SMILES: c1ccc(-n2c3ccccc3c3cc(-c4cccc(N(c5cccc(-c6cccc7c6ccc6ccccc67)c5)c5cc6ccccc6c6ccccc56)c4)ccc32)cc1 ; c1ccc(-n2c3ccccc3c3c(-c4ccc(N(c5cccc(-c6cccc7c6ccc6ccccc67)c5)c5cc6ccccc6c6ccccc56)cc4)cccc32)cc1 ; c1cc(-c2cccc3c2ccc2ccccc23)cc(N(c2ccccc2-n2c3ccccc3c3ccccc32)c2cc3ccccc3c3ccccc23)c1]

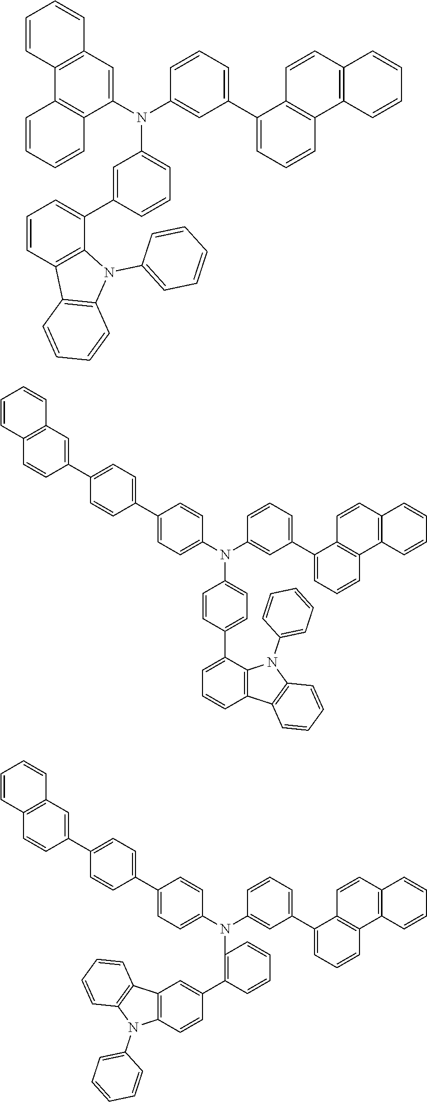 [SMILES: c1ccc(-n2c3ccccc3c3cccc(-c4cccc(N(c5cccc(-c6cccc7c6ccc6ccccc67)c5)c5cc6ccccc6c6ccccc56)c4)c32)cc1 ; c1ccc(-n2c3ccccc3c3cccc(-c4ccc(N(c5ccc(-c6ccc(-c7ccc8ccccc8c7)cc6)cc5)c5cccc(-c6cccc7c6ccc6ccccc67)c5)cc4)c32)cc1 ; c1ccc(-n2c3ccccc3c3cc(-c4ccccc4N(c4ccc(-c5ccc(-c6ccc7ccccc7c6)cc5)cc4)c4cccc(-c5cccc6c5ccc5ccccc56)c4)ccc32)cc1]

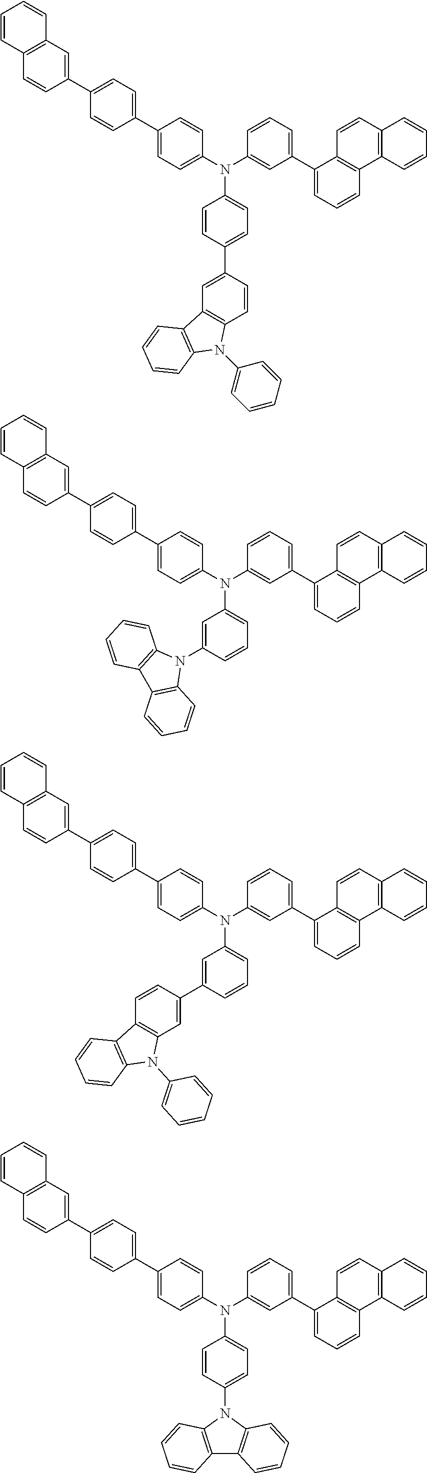 [SMILES: c1ccc(-n2c3ccccc3c3cc(-c4ccc(N(c5ccc(-c6ccc(-c7ccc8ccccc8c7)cc6)cc5)c5cccc(-c6cccc7c6ccc6ccccc67)c5)cc4)ccc32)cc1 ; c1cc(-c2cccc3c2ccc2ccccc23)cc(N(c2ccc(-c3ccc(-c4ccc5ccccc5c4)cc3)cc2)c2cccc(-n3c4ccccc4c4ccccc43)c2)c1 ; c1ccc(-n2c3ccccc3c3ccc(-c4cccc(N(c5ccc(-c6ccc(-c7ccc8ccccc8c7)cc6)cc5)c5cccc(-c6cccc7c6ccc6ccccc67)c5)c4)cc32)cc1 ; c1cc(-c2cccc3c2ccc2ccccc23)cc(N(c2ccc(-c3ccc(-c4ccc5ccccc5c4)cc3)cc2)c2ccc(-n3c4ccccc4c4ccccc43)cc2)c1]

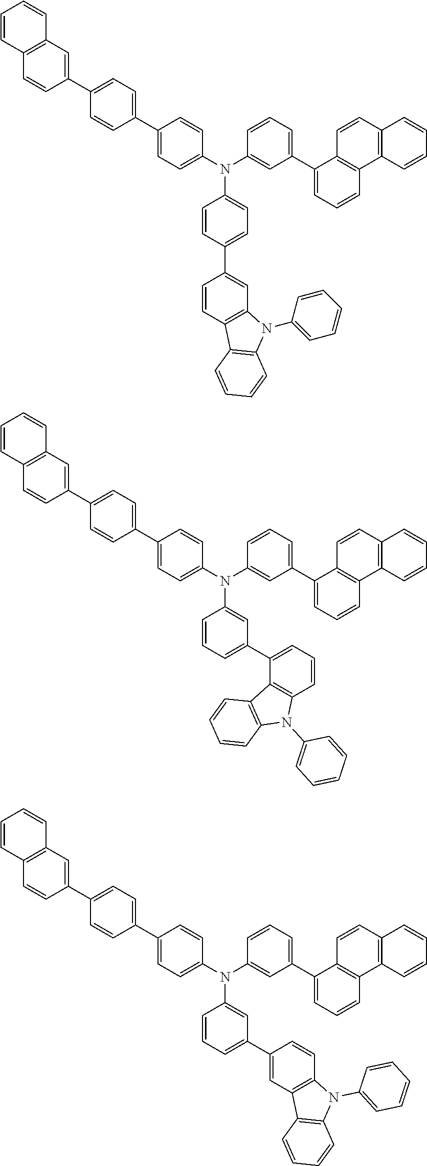 [SMILES: c1ccc(-n2c3ccccc3c3ccc(-c4ccc(N(c5ccc(-c6ccc(-c7ccc8ccccc8c7)cc6)cc5)c5cccc(-c6cccc7c6ccc6ccccc67)c5)cc4)cc32)cc1 ; c1ccc(-n2c3ccccc3c3c(-c4cccc(N(c5ccc(-c6ccc(-c7ccc8ccccc8c7)cc6)cc5)c5cccc(-c6cccc7c6ccc6ccccc67)c5)c4)cccc32)cc1 ; c1ccc(-n2c3ccccc3c3cc(-c4cccc(N(c5ccc(-c6ccc(-c7ccc8ccccc8c7)cc6)cc5)c5cccc(-c6cccc7c6ccc6ccccc67)c5)c4)ccc32)cc1]

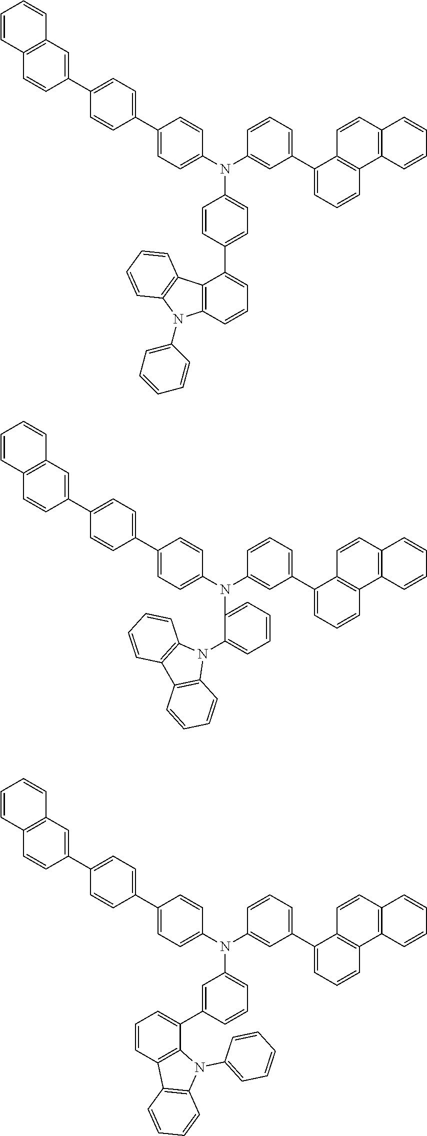 [SMILES: c1ccc(-n2c3ccccc3c3c(-c4ccc(N(c5ccc(-c6ccc(-c7ccc8ccccc8c7)cc6)cc5)c5cccc(-c6cccc7c6ccc6ccccc67)c5)cc4)cccc32)cc1 ; c1cc(-c2cccc3c2ccc2ccccc23)cc(N(c2ccc(-c3ccc(-c4ccc5ccccc5c4)cc3)cc2)c2ccccc2-n2c3ccccc3c3ccccc32)c1 ; c1ccc(-n2c3ccccc3c3cccc(-c4cccc(N(c5ccc(-c6ccc(-c7ccc8ccccc8c7)cc6)cc5)c5cccc(-c6cccc7c6ccc6ccccc67)c5)c4)c32)cc1]

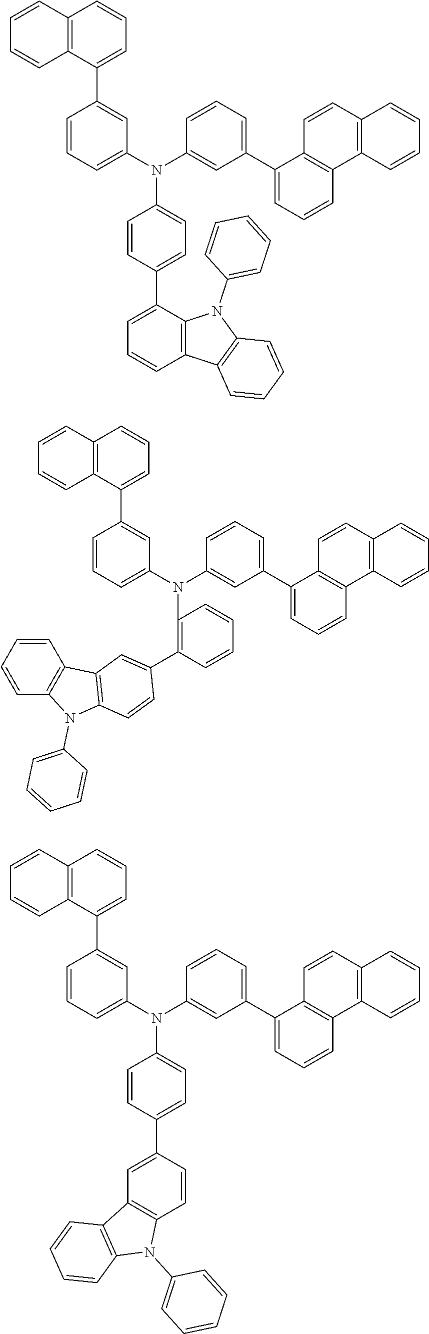

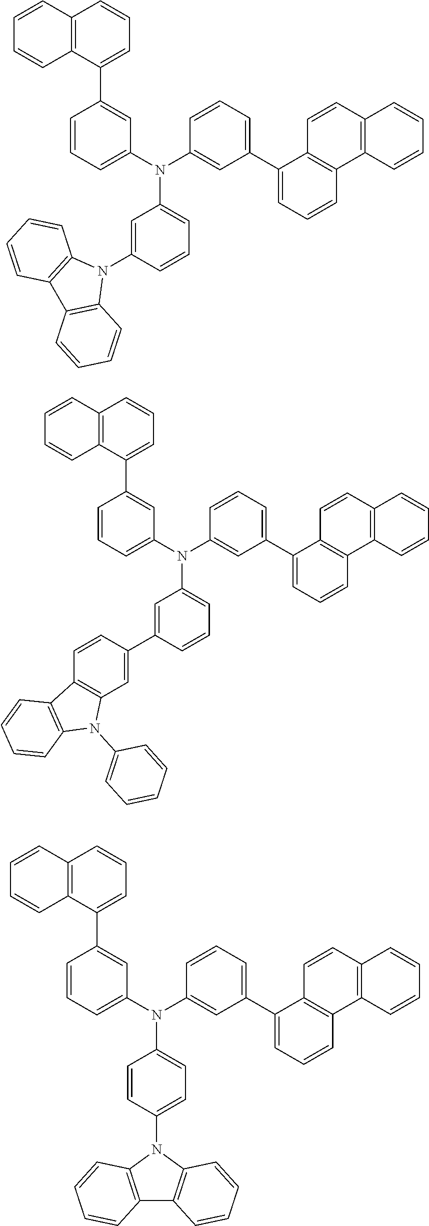

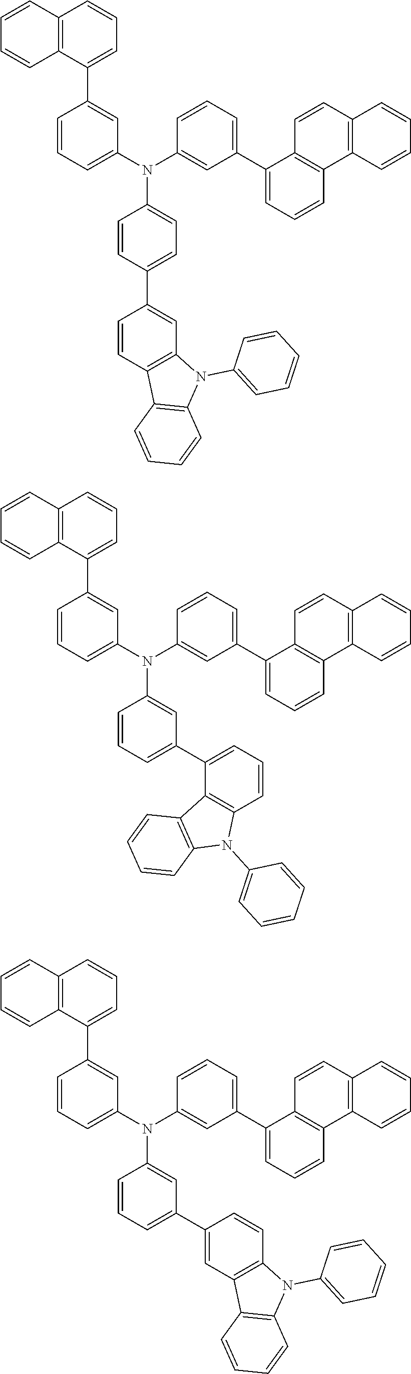 [SMILES: c1ccc(-n2c3ccccc3c3ccc(-c4ccc(N(c5cccc(-c6cccc7ccccc67)c5)c5cccc(-c6cccc7c6ccc6ccccc67)c5)cc4)cc32)cc1 ; c1ccc(-n2c3ccccc3c3c(-c4cccc(N(c5cccc(-c6cccc7ccccc67)c5)c5cccc(-c6cccc7c6ccc6ccccc67)c5)c4)cccc32)cc1 ; c1ccc(-n2c3ccccc3c3cc(-c4cccc(N(c5cccc(-c6cccc7ccccc67)c5)c5cccc(-c6cccc7c6ccc6ccccc67)c5)c4)ccc32)cc1]

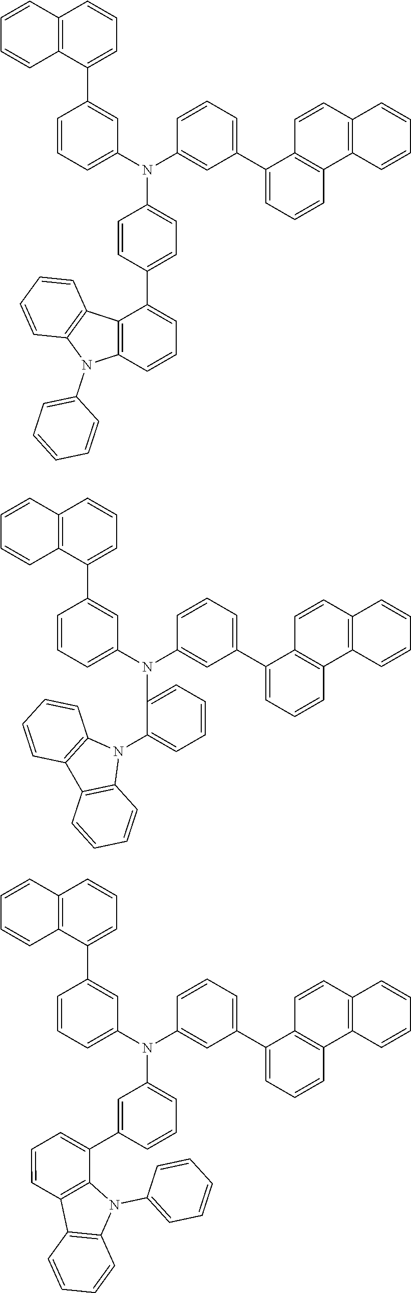

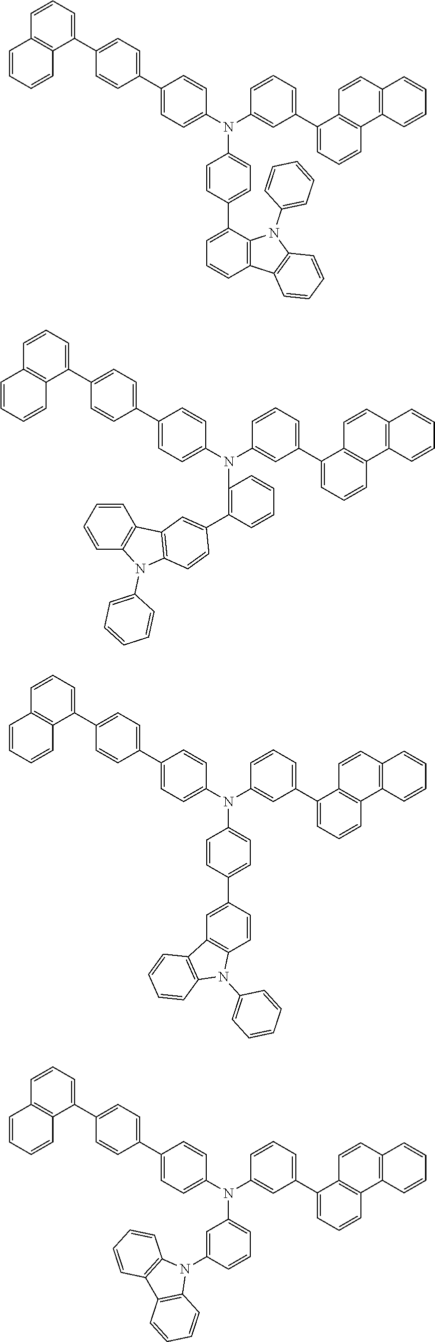 [SMILES: c1ccc(-n2c3ccccc3c3cccc(-c4ccc(N(c5ccc(-c6ccc(-c7cccc8ccccc78)cc6)cc5)c5cccc(-c6cccc7c6ccc6ccccc67)c5)cc4)c32)cc1 ; c1ccc(-n2c3ccccc3c3cc(-c4ccccc4N(c4ccc(-c5ccc(-c6cccc7ccccc67)cc5)cc4)c4cccc(-c5cccc6c5ccc5ccccc56)c4)ccc32)cc1 ; c1ccc(-n2c3ccccc3c3cc(-c4ccc(N(c5ccc(-c6ccc(-c7cccc8ccccc78)cc6)cc5)c5cccc(-c6cccc7c6ccc6ccccc67)c5)cc4)ccc32)cc1 ; c1cc(-c2cccc3c2ccc2ccccc23)cc(N(c2ccc(-c3ccc(-c4cccc5ccccc45)cc3)cc2)c2cccc(-n3c4ccccc4c4ccccc43)c2)c1]

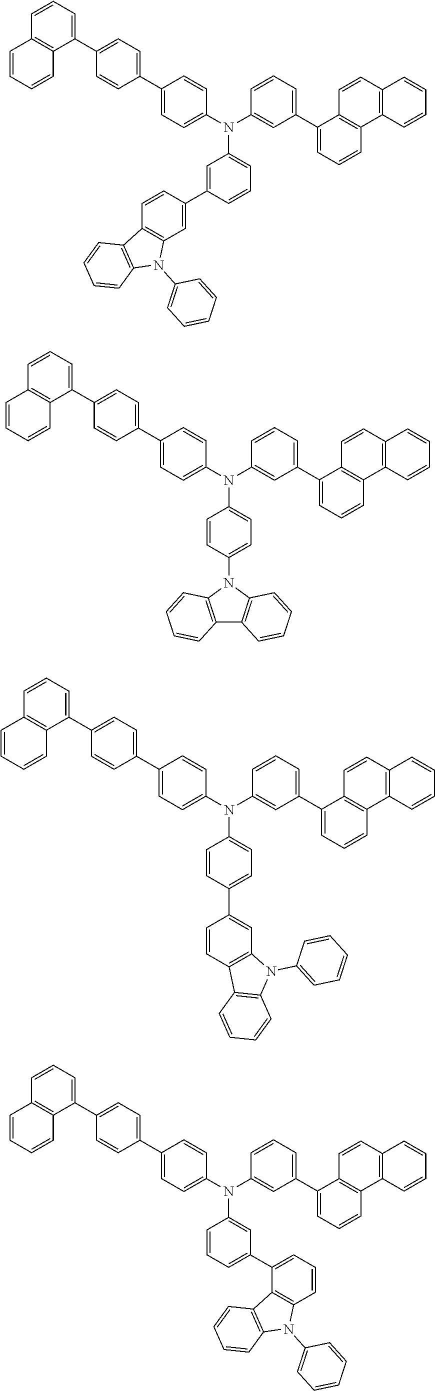 [SMILES: c1ccc(-n2c3ccccc3c3ccc(-c4cccc(N(c5ccc(-c6ccc(-c7cccc8ccccc78)cc6)cc5)c5cccc(-c6cccc7c6ccc6ccccc67)c5)c4)cc32)cc1 ; c1cc(-c2cccc3c2ccc2ccccc23)cc(N(c2ccc(-c3ccc(-c4cccc5ccccc45)cc3)cc2)c2ccc(-n3c4ccccc4c4ccccc43)cc2)c1 ; c1ccc(-n2c3ccccc3c3ccc(-c4ccc(N(c5ccc(-c6ccc(-c7cccc8ccccc78)cc6)cc5)c5cccc(-c6cccc7c6ccc6ccccc67)c5)cc4)cc32)cc1 ; c1ccc(-n2c3ccccc3c3c(-c4cccc(N(c5ccc(-c6ccc(-c7cccc8ccccc78)cc6)cc5)c5cccc(-c6cccc7c6ccc6ccccc67)c5)c4)cccc32)cc1]

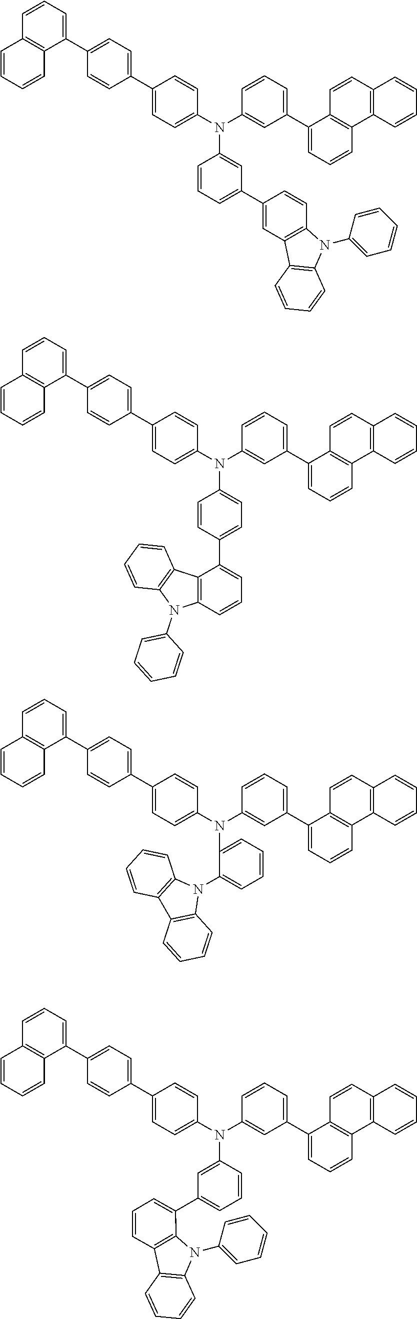 [SMILES: c1ccc(-n2c3ccccc3c3cc(-c4cccc(N(c5ccc(-c6ccc(-c7cccc8ccccc78)cc6)cc5)c5cccc(-c6cccc7c6ccc6ccccc67)c5)c4)ccc32)cc1 ; c1ccc(-n2c3ccccc3c3c(-c4ccc(N(c5ccc(-c6ccc(-c7cccc8ccccc78)cc6)cc5)c5cccc(-c6cccc7c6ccc6ccccc67)c5)cc4)cccc32)cc1 ; c1cc(-c2cccc3c2ccc2ccccc23)cc(N(c2ccc(-c3ccc(-c4cccc5ccccc45)cc3)cc2)c2ccccc2-n2c3ccccc3c3ccccc32)c1 ; c1ccc(-n2c3ccccc3c3cccc(-c4cccc(N(c5ccc(-c6ccc(-c7cccc8ccccc78)cc6)cc5)c5cccc(-c6cccc7c6ccc6ccccc67)c5)c4)c32)cc1]

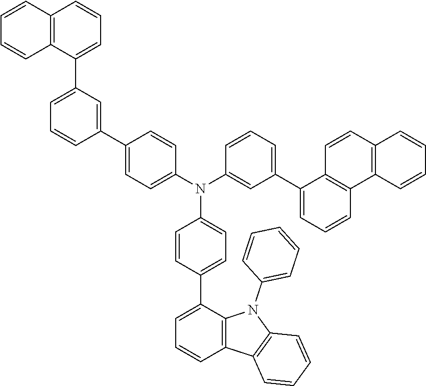 [SMILES: c1ccc(-n2c3ccccc3c3cccc(-c4ccc(N(c5ccc(-c6cccc(-c7cccc8ccccc78)c6)cc5)c5cccc(-c6cccc7c6ccc6ccccc67)c5)cc4)c32)cc1]

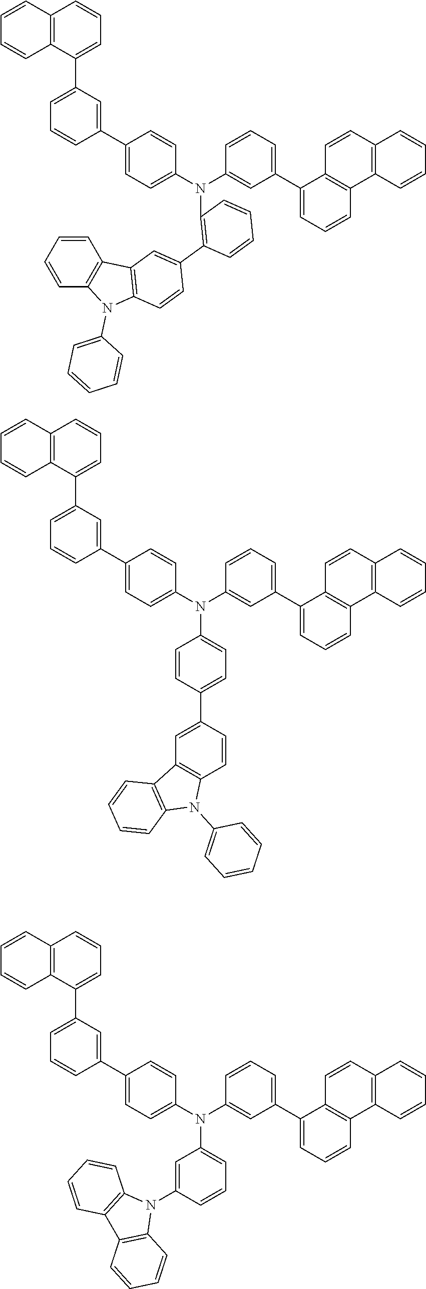

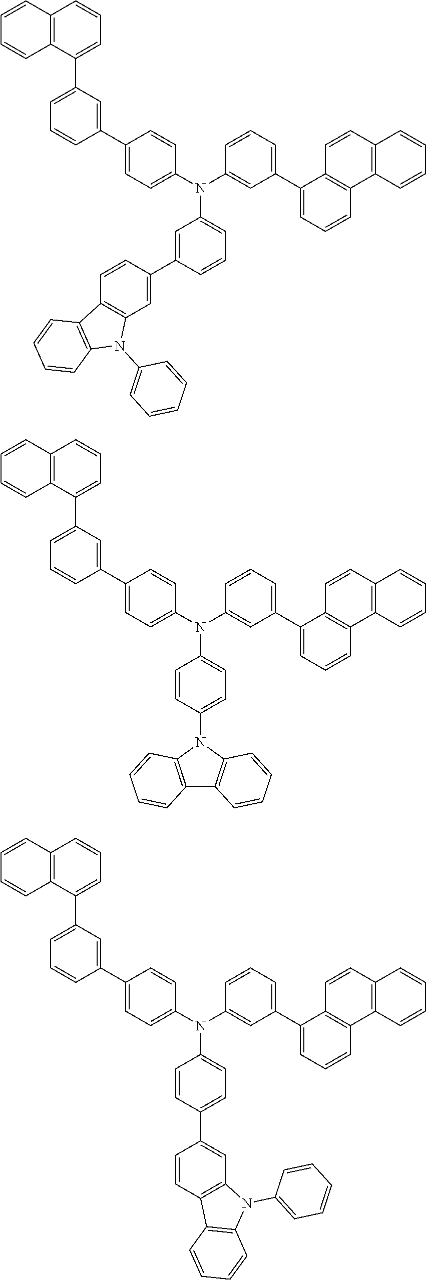 [SMILES: c1ccc(-n2c3ccccc3c3ccc(-c4cccc(N(c5ccc(-c6cccc(-c7cccc8ccccc78)c6)cc5)c5cccc(-c6cccc7c6ccc6ccccc67)c5)c4)cc32)cc1 ; c1cc(-c2ccc(N(c3ccc(-n4c5ccccc5c5ccccc54)cc3)c3cccc(-c4cccc5c4ccc4ccccc45)c3)cc2)cc(-c2cccc3ccccc23)c1 ; c1ccc(-n2c3ccccc3c3ccc(-c4ccc(N(c5ccc(-c6cccc(-c7cccc8ccccc78)c6)cc5)c5cccc(-c6cccc7c6ccc6ccccc67)c5)cc4)cc32)cc1]

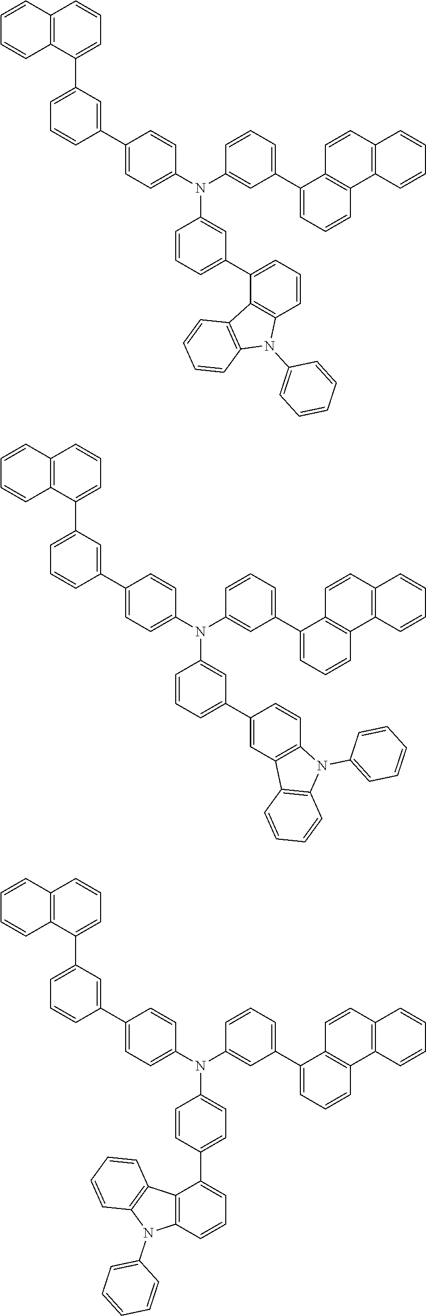 [SMILES: c1ccc(-n2c3ccccc3c3c(-c4cccc(N(c5ccc(-c6cccc(-c7cccc8ccccc78)c6)cc5)c5cccc(-c6cccc7c6ccc6ccccc67)c5)c4)cccc32)cc1 ; c1ccc(-n2c3ccccc3c3cc(-c4cccc(N(c5ccc(-c6cccc(-c7cccc8ccccc78)c6)cc5)c5cccc(-c6cccc7c6ccc6ccccc67)c5)c4)ccc32)cc1 ; c1ccc(-n2c3ccccc3c3c(-c4ccc(N(c5ccc(-c6cccc(-c7cccc8ccccc78)c6)cc5)c5cccc(-c6cccc7c6ccc6ccccc67)c5)cc4)cccc32)cc1]

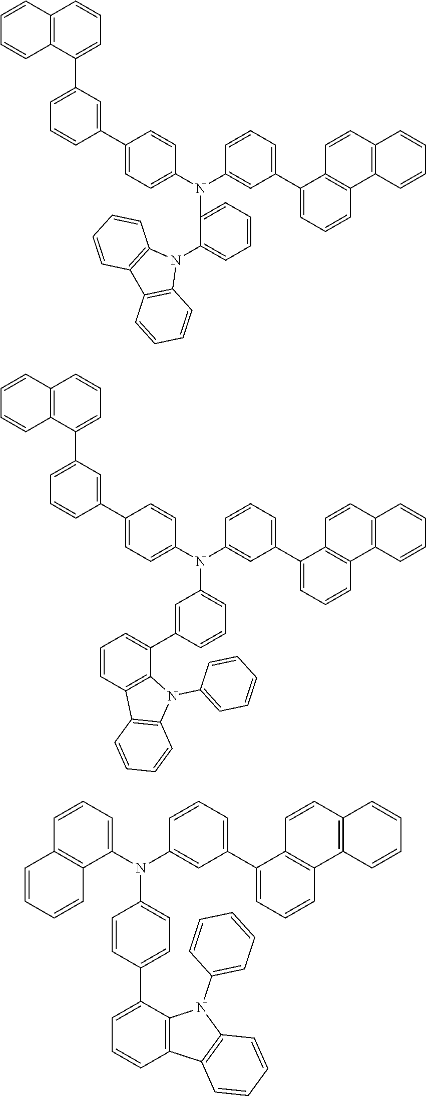

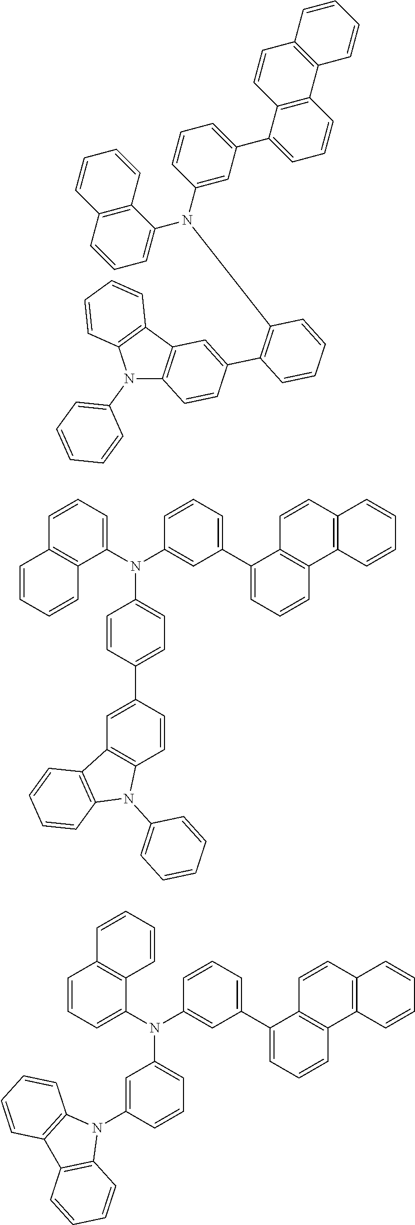

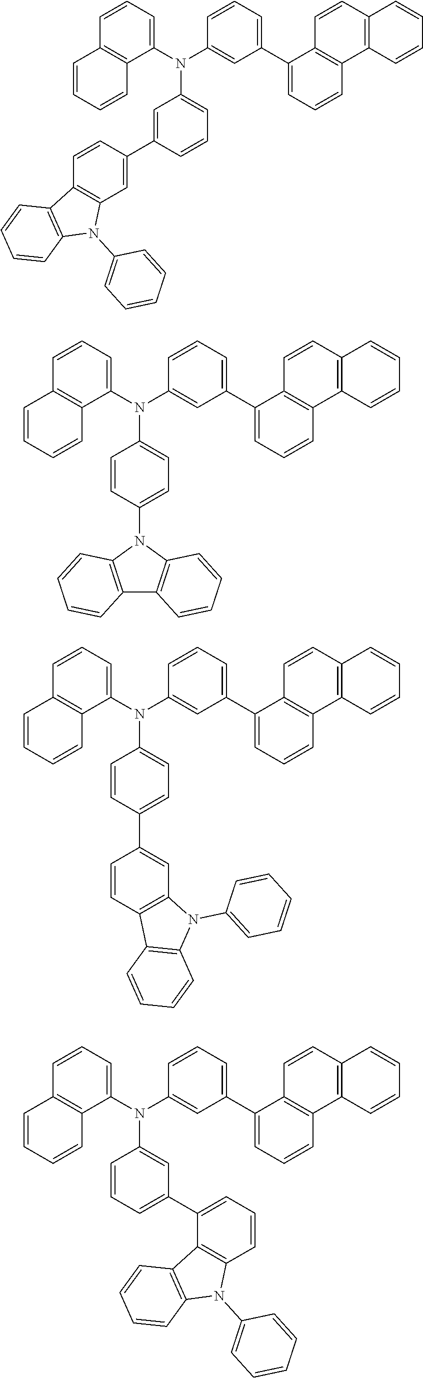

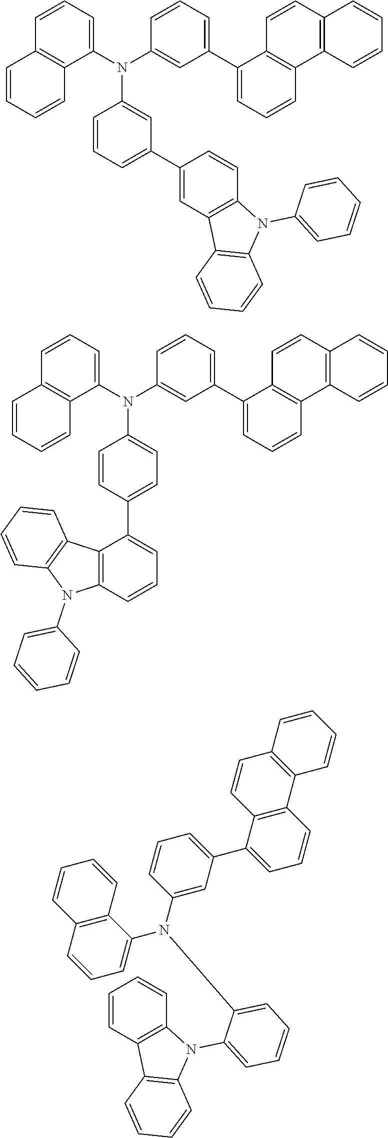

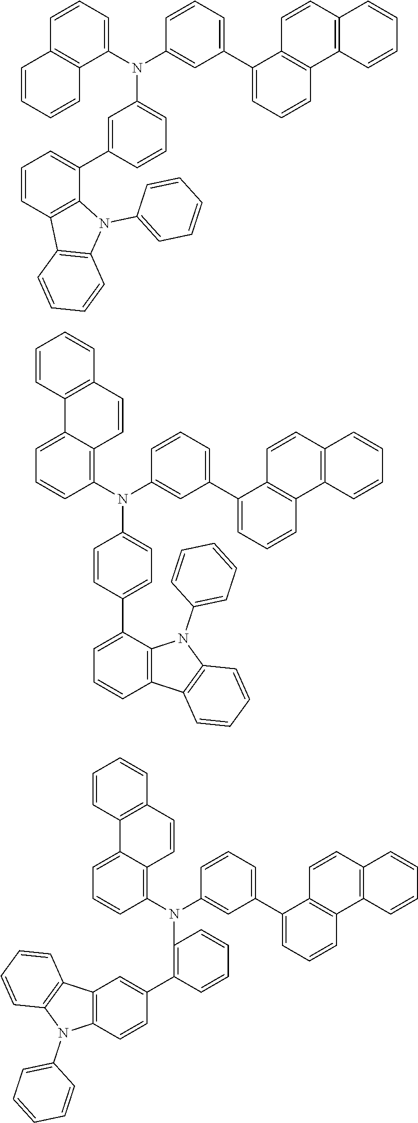

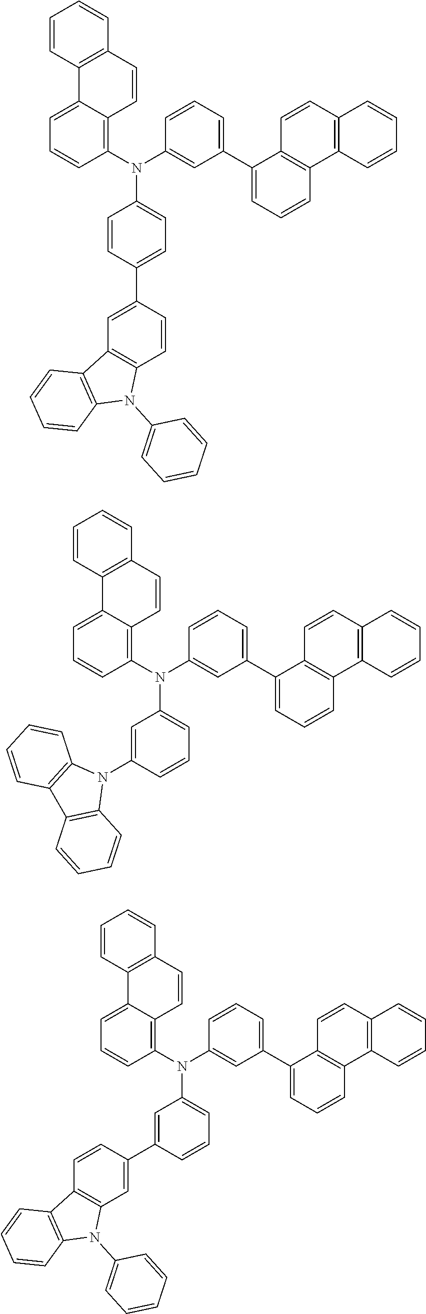

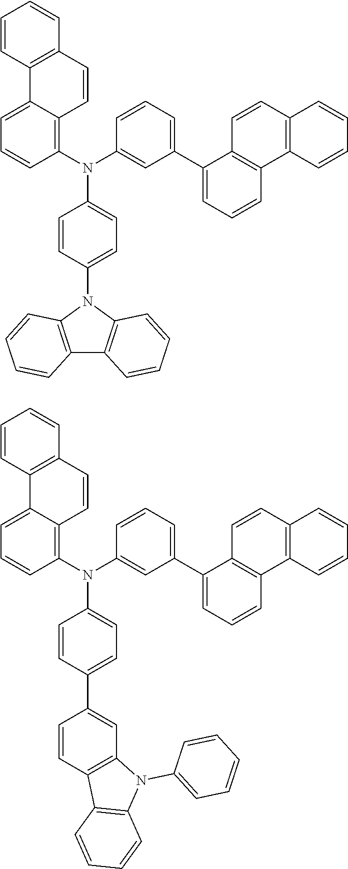

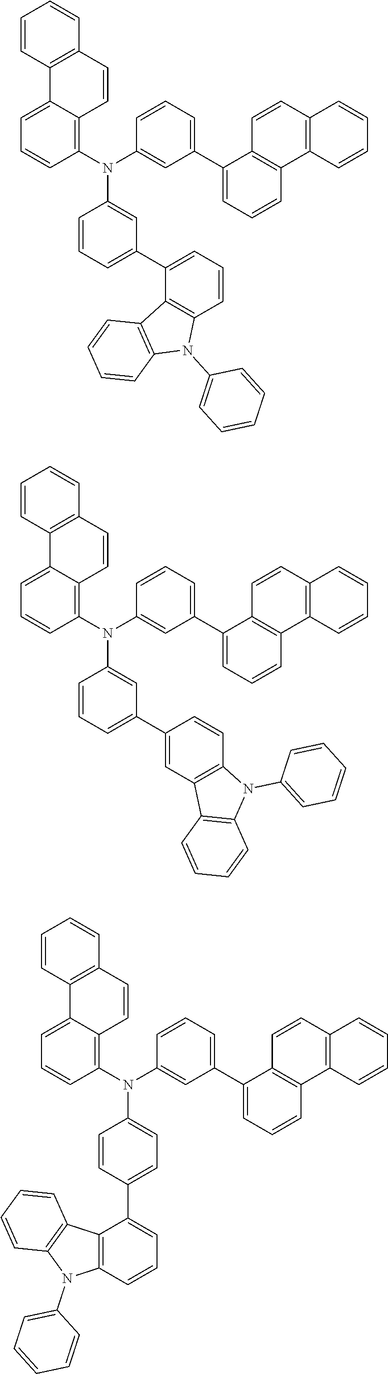 [SMILES: c1ccc(-n2c3ccccc3c3c(-c4cccc(N(c5cccc(-c6cccc7c6ccc6ccccc67)c5)c5cccc6c5ccc5ccccc56)c4)cccc32)cc1 ; c1ccc(-n2c3ccccc3c3cc(-c4cccc(N(c5cccc(-c6cccc7c6ccc6ccccc67)c5)c5cccc6c5ccc5ccccc56)c4)ccc32)cc1 ; c1ccc(-n2c3ccccc3c3c(-c4ccc(N(c5cccc(-c6cccc7c6ccc6ccccc67)c5)c5cccc6c5ccc5ccccc56)cc4)cccc32)cc1]

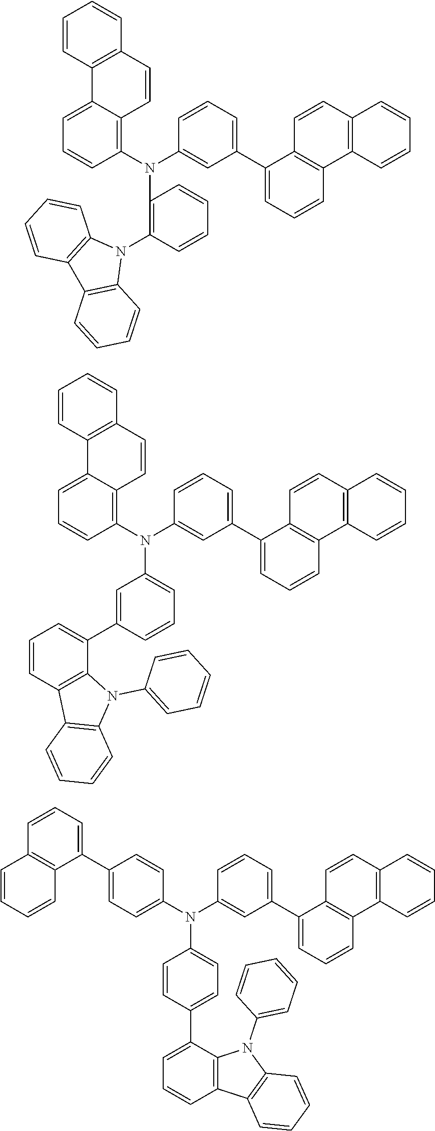

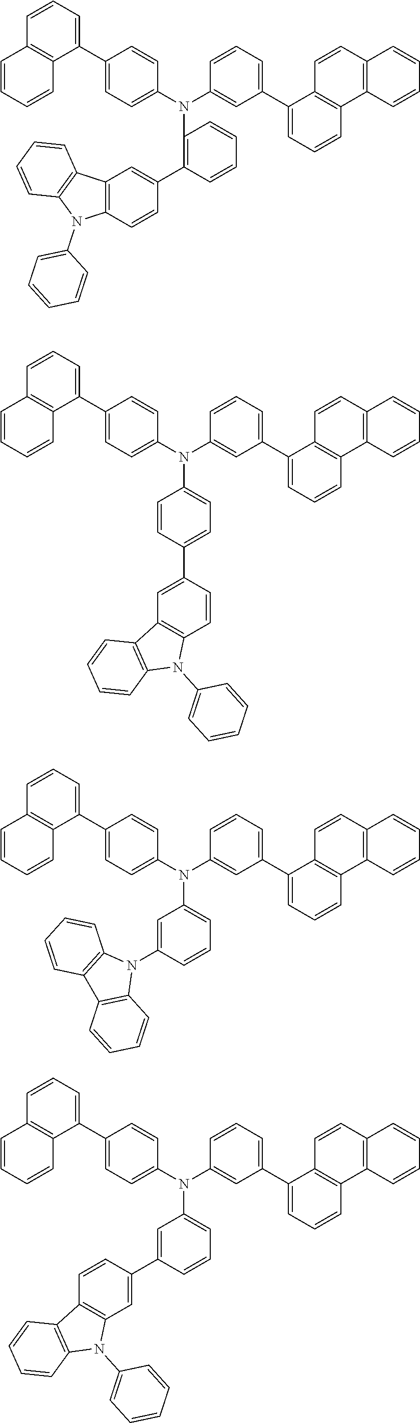 [SMILES: c1ccc(-n2c3ccccc3c3cc(-c4ccccc4N(c4ccc(-c5cccc6ccccc56)cc4)c4cccc(-c5cccc6c5ccc5ccccc56)c4)ccc32)cc1 ; c1ccc(-n2c3ccccc3c3cc(-c4ccc(N(c5ccc(-c6cccc7ccccc67)cc5)c5cccc(-c6cccc7c6ccc6ccccc67)c5)cc4)ccc32)cc1 ; c1cc(-c2cccc3c2ccc2ccccc23)cc(N(c2ccc(-c3cccc4ccccc34)cc2)c2cccc(-n3c4ccccc4c4ccccc43)c2)c1 ; c1ccc(-n2c3ccccc3c3ccc(-c4cccc(N(c5ccc(-c6cccc7ccccc67)cc5)c5cccc(-c6cccc7c6ccc6ccccc67)c5)c4)cc32)cc1]

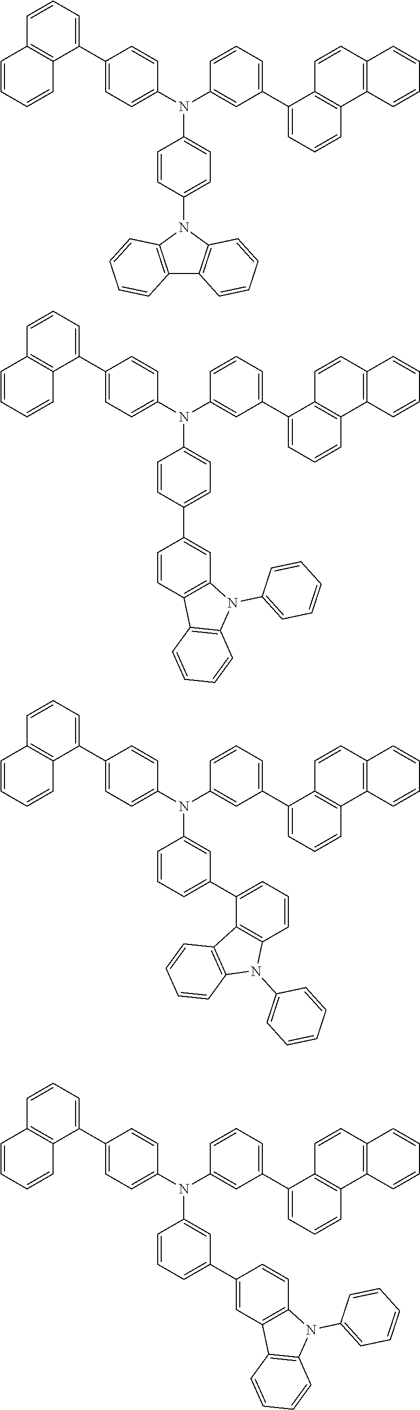 [SMILES: c1cc(-c2cccc3c2ccc2ccccc23)cc(N(c2ccc(-c3cccc4ccccc34)cc2)c2ccc(-n3c4ccccc4c4ccccc43)cc2)c1 ; c1ccc(-n2c3ccccc3c3ccc(-c4ccc(N(c5ccc(-c6cccc7ccccc67)cc5)c5cccc(-c6cccc7c6ccc6ccccc67)c5)cc4)cc32)cc1 ; c1ccc(-n2c3ccccc3c3c(-c4cccc(N(c5ccc(-c6cccc7ccccc67)cc5)c5cccc(-c6cccc7c6ccc6ccccc67)c5)c4)cccc32)cc1 ; c1ccc(-n2c3ccccc3c3cc(-c4cccc(N(c5ccc(-c6cccc7ccccc67)cc5)c5cccc(-c6cccc7c6ccc6ccccc67)c5)c4)ccc32)cc1]

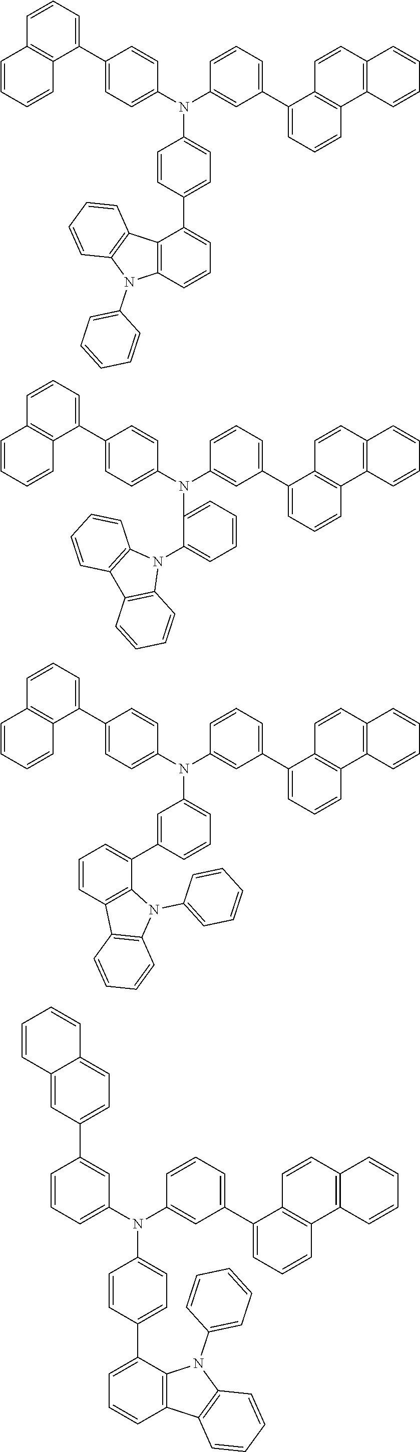

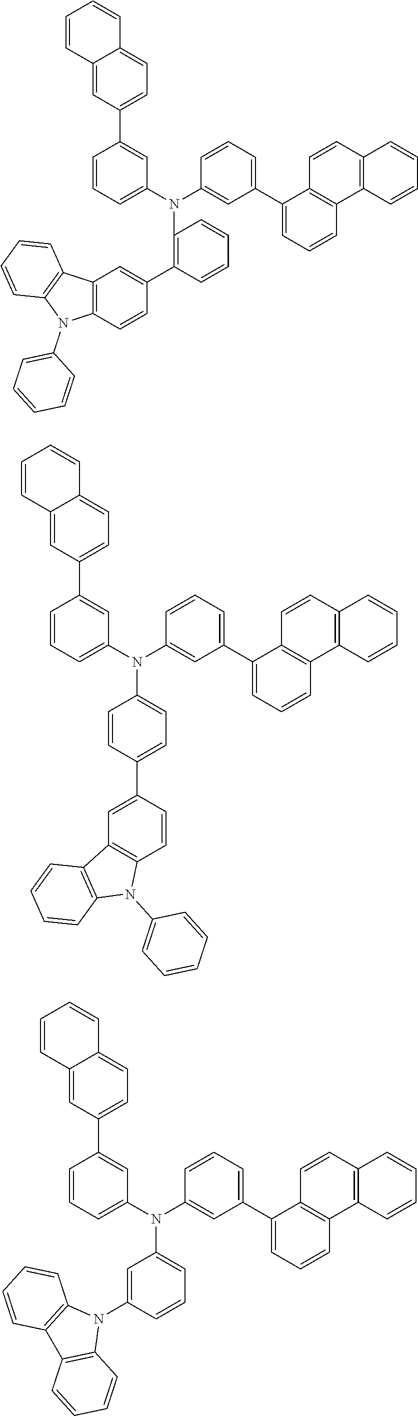

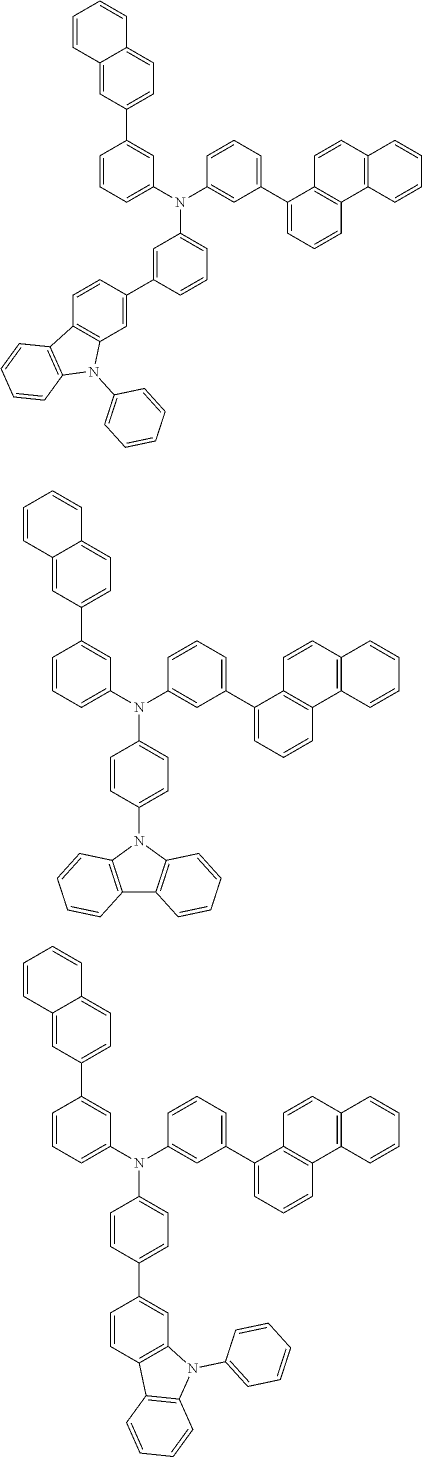

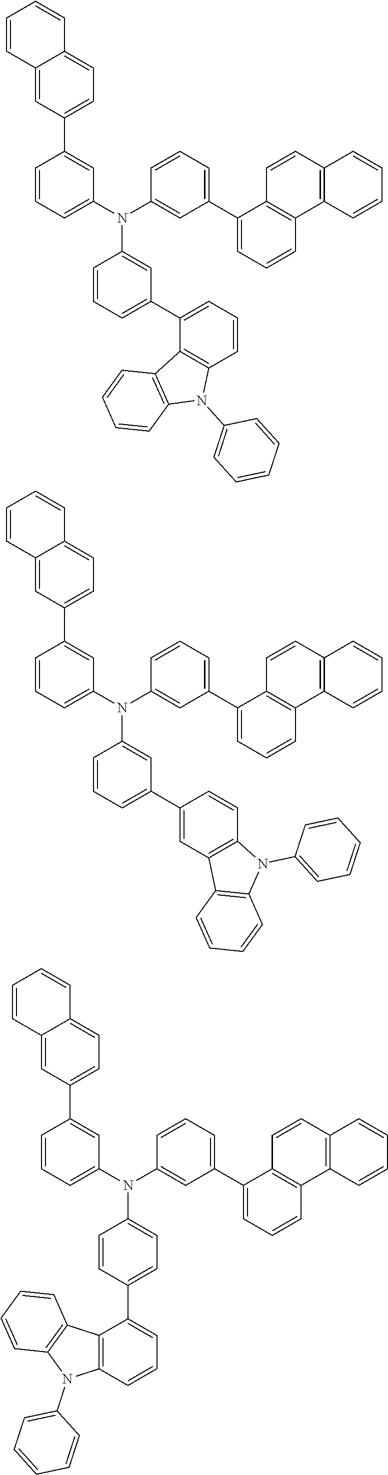 [SMILES: c1ccc(-n2c3ccccc3c3c(-c4cccc(N(c5cccc(-c6ccc7ccccc7c6)c5)c5cccc(-c6cccc7c6ccc6ccccc67)c5)c4)cccc32)cc1 ; c1ccc(-n2c3ccccc3c3cc(-c4cccc(N(c5cccc(-c6ccc7ccccc7c6)c5)c5cccc(-c6cccc7c6ccc6ccccc67)c5)c4)ccc32)cc1 ; c1ccc(-n2c3ccccc3c3c(-c4ccc(N(c5cccc(-c6ccc7ccccc7c6)c5)c5cccc(-c6cccc7c6ccc6ccccc67)c5)cc4)cccc32)cc1]

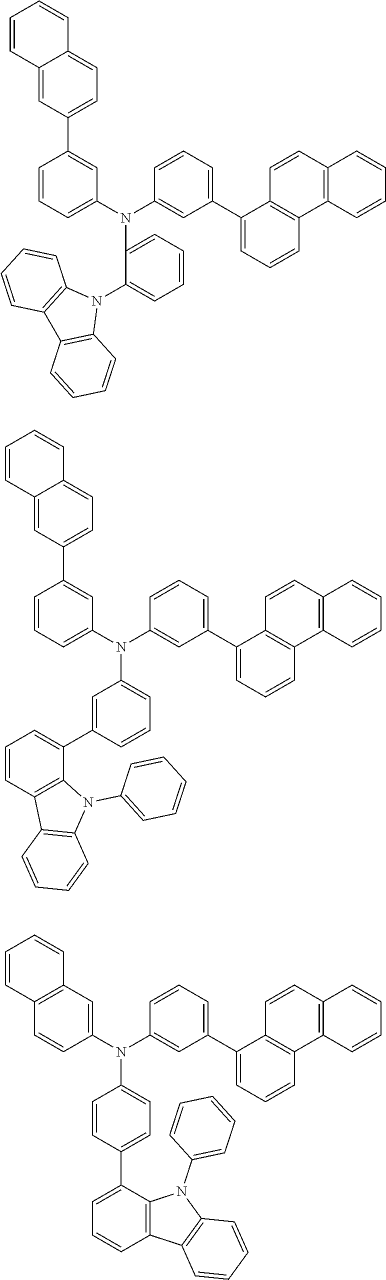 [SMILES: c1cc(-c2ccc3ccccc3c2)cc(N(c2cccc(-c3cccc4c3ccc3ccccc34)c2)c2ccccc2-n2c3ccccc3c3ccccc32)c1 ; c1ccc(-n2c3ccccc3c3cccc(-c4cccc(N(c5cccc(-c6ccc7ccccc7c6)c5)c5cccc(-c6cccc7c6ccc6ccccc67)c5)c4)c32)cc1 ; c1ccc(-n2c3ccccc3c3cccc(-c4ccc(N(c5cccc(-c6cccc7c6ccc6ccccc67)c5)c5ccc6ccccc6c5)cc4)c32)cc1]

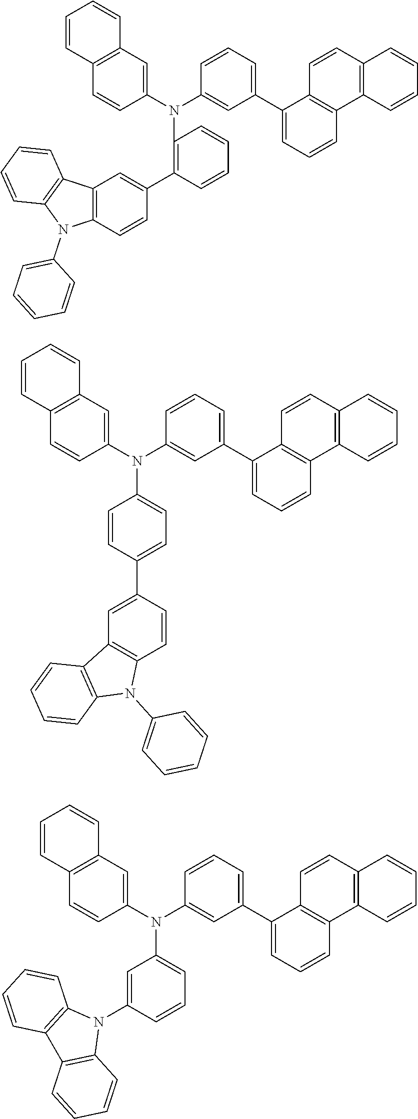

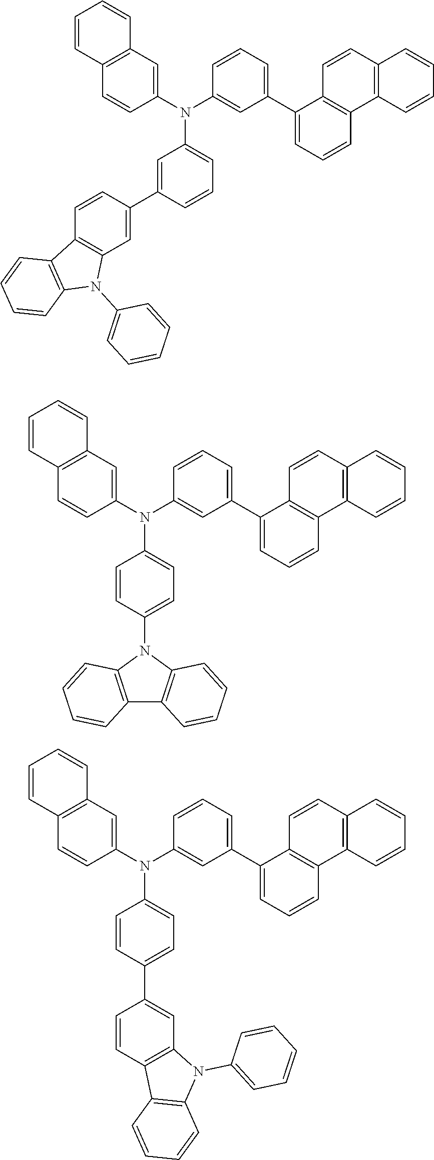

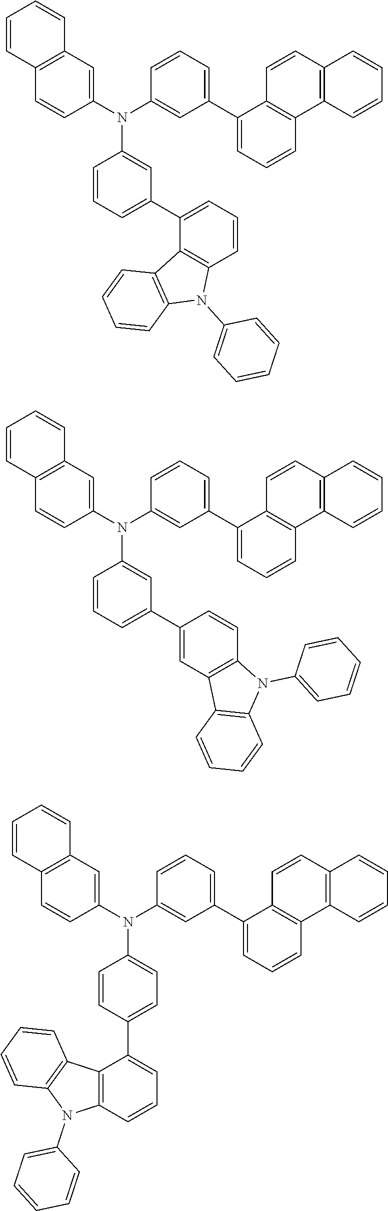 [SMILES: c1ccc(-n2c3ccccc3c3c(-c4cccc(N(c5cccc(-c6cccc7c6ccc6ccccc67)c5)c5ccc6ccccc6c5)c4)cccc32)cc1 ; c1ccc(-n2c3ccccc3c3cc(-c4cccc(N(c5cccc(-c6cccc7c6ccc6ccccc67)c5)c5ccc6ccccc6c5)c4)ccc32)cc1 ; c1ccc(-n2c3ccccc3c3c(-c4ccc(N(c5cccc(-c6cccc7c6ccc6ccccc67)c5)c5ccc6ccccc6c5)cc4)cccc32)cc1]

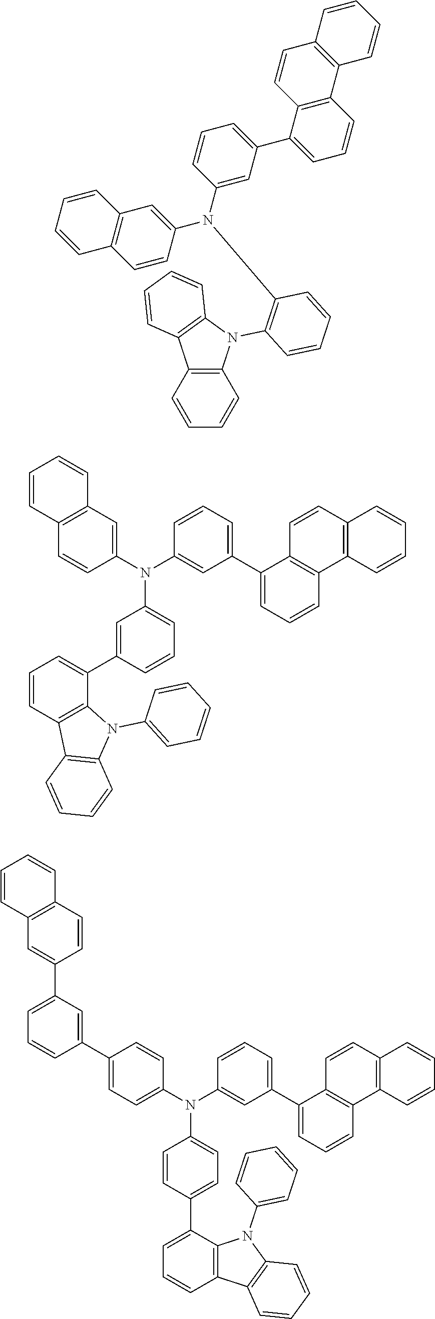 [SMILES: c1cc(-c2cccc3c2ccc2ccccc23)cc(N(c2ccc3ccccc3c2)c2ccccc2-n2c3ccccc3c3ccccc32)c1 ; c1ccc(-n2c3ccccc3c3cccc(-c4cccc(N(c5cccc(-c6cccc7c6ccc6ccccc67)c5)c5ccc6ccccc6c5)c4)c32)cc1 ; c1ccc(-n2c3ccccc3c3cccc(-c4ccc(N(c5ccc(-c6cccc(-c7ccc8ccccc8c7)c6)cc5)c5cccc(-c6cccc7c6ccc6ccccc67)c5)cc4)c32)cc1]

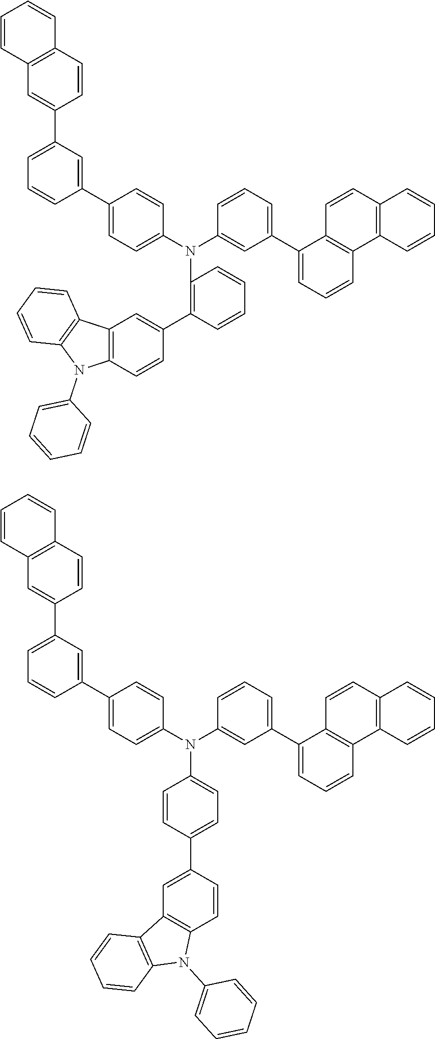

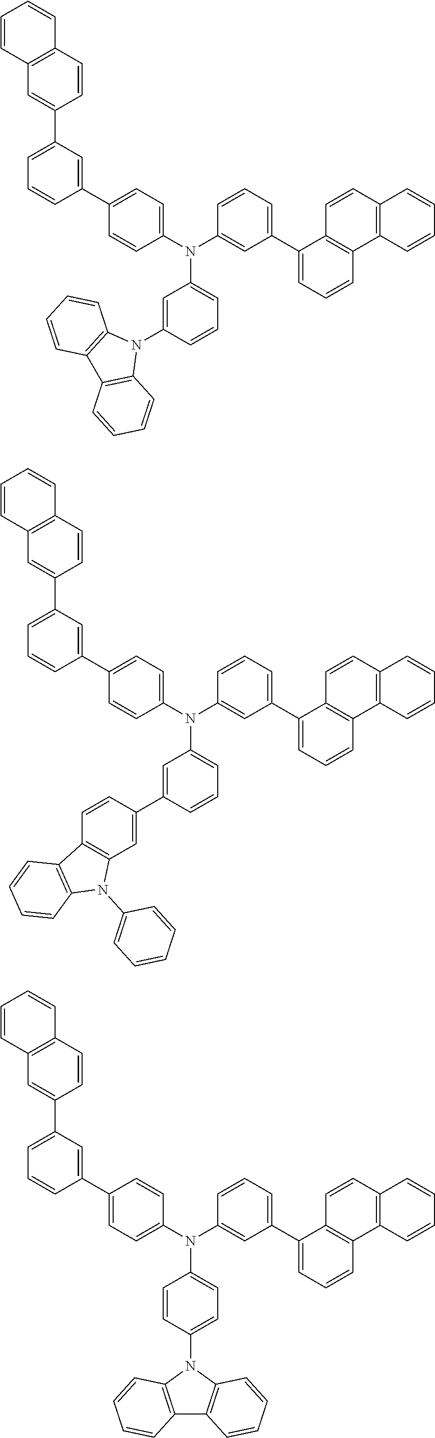 [SMILES: c1cc(-c2ccc(N(c3cccc(-c4cccc5c4ccc4ccccc45)c3)c3cccc(-n4c5ccccc5c5ccccc54)c3)cc2)cc(-c2ccc3ccccc3c2)c1 ; c1ccc(-n2c3ccccc3c3ccc(-c4cccc(N(c5ccc(-c6cccc(-c7ccc8ccccc8c7)c6)cc5)c5cccc(-c6cccc7c6ccc6ccccc67)c5)c4)cc32)cc1 ; c1cc(-c2ccc(N(c3ccc(-n4c5ccccc5c5ccccc54)cc3)c3cccc(-c4cccc5c4ccc4ccccc45)c3)cc2)cc(-c2ccc3ccccc3c2)c1]

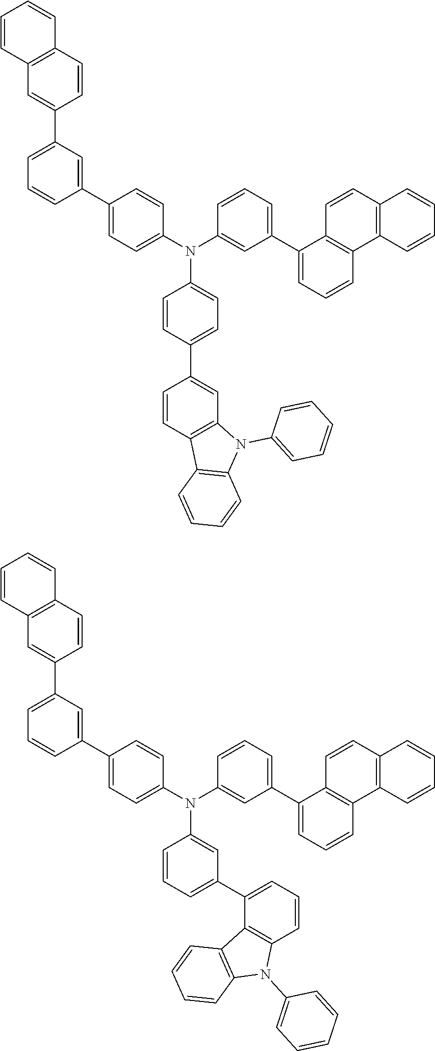 [SMILES: c1ccc(-n2c3ccccc3c3ccc(-c4ccc(N(c5ccc(-c6cccc(-c7ccc8ccccc8c7)c6)cc5)c5cccc(-c6cccc7c6ccc6ccccc67)c5)cc4)cc32)cc1 ; c1ccc(-n2c3ccccc3c3c(-c4cccc(N(c5ccc(-c6cccc(-c7ccc8ccccc8c7)c6)cc5)c5cccc(-c6cccc7c6ccc6ccccc67)c5)c4)cccc32)cc1]

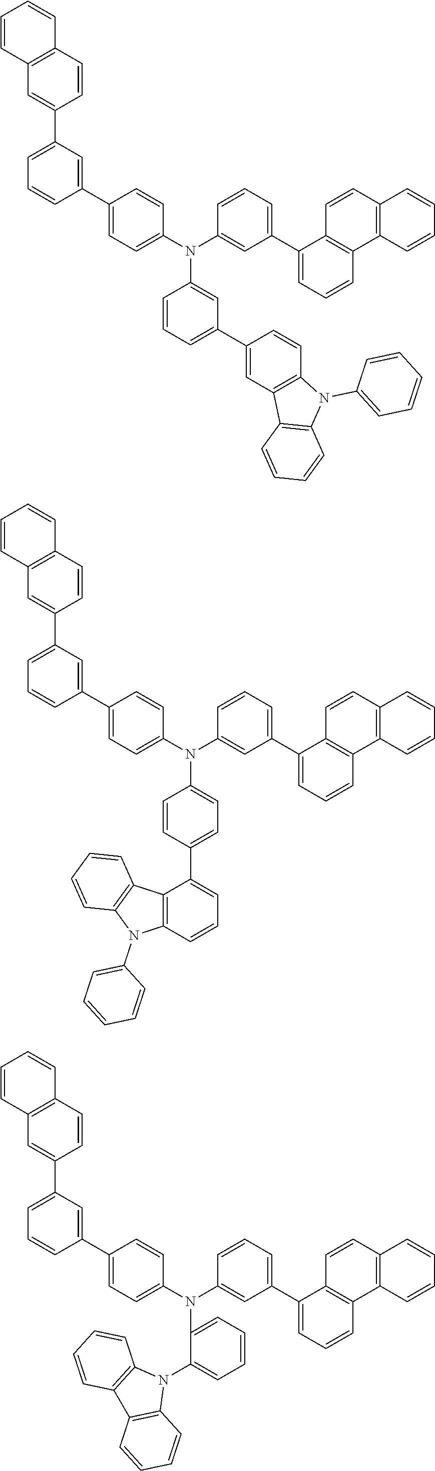 [SMILES: c1ccc(-n2c3ccccc3c3cc(-c4cccc(N(c5ccc(-c6cccc(-c7ccc8ccccc8c7)c6)cc5)c5cccc(-c6cccc7c6ccc6ccccc67)c5)c4)ccc32)cc1 ; c1ccc(-n2c3ccccc3c3c(-c4ccc(N(c5ccc(-c6cccc(-c7ccc8ccccc8c7)c6)cc5)c5cccc(-c6cccc7c6ccc6ccccc67)c5)cc4)cccc32)cc1 ; c1cc(-c2ccc(N(c3cccc(-c4cccc5c4ccc4ccccc45)c3)c3ccccc3-n3c4ccccc4c4ccccc43)cc2)cc(-c2ccc3ccccc3c2)c1]

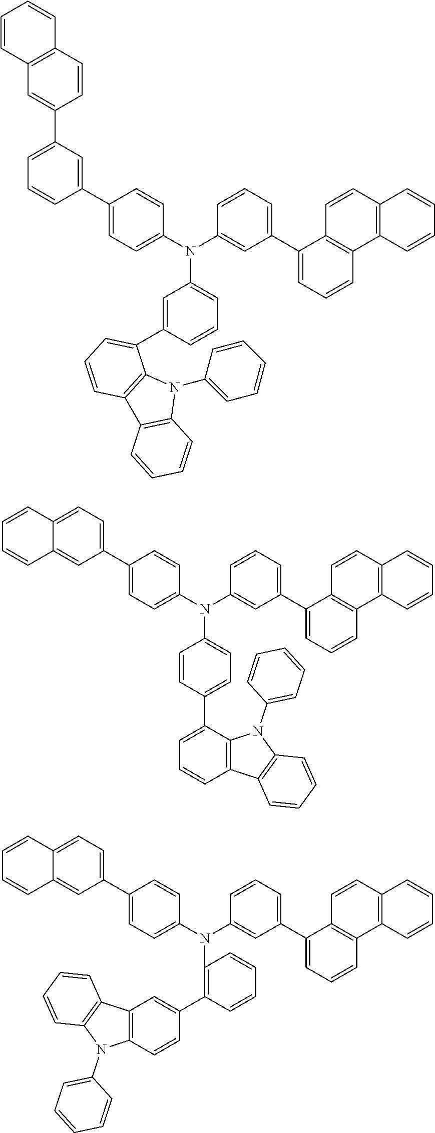 [SMILES: c1ccc(-n2c3ccccc3c3cccc(-c4cccc(N(c5ccc(-c6cccc(-c7ccc8ccccc8c7)c6)cc5)c5cccc(-c6cccc7c6ccc6ccccc67)c5)c4)c32)cc1 ; c1ccc(-n2c3ccccc3c3cccc(-c4ccc(N(c5ccc(-c6ccc7ccccc7c6)cc5)c5cccc(-c6cccc7c6ccc6ccccc67)c5)cc4)c32)cc1 ; c1ccc(-n2c3ccccc3c3cc(-c4ccccc4N(c4ccc(-c5ccc6ccccc6c5)cc4)c4cccc(-c5cccc6c5ccc5ccccc56)c4)ccc32)cc1]

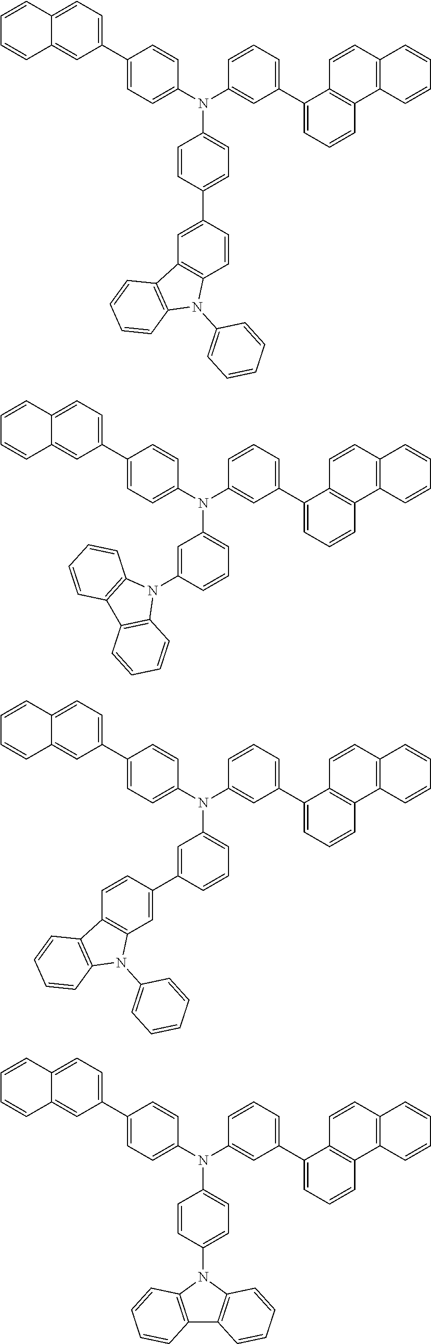 [SMILES: c1ccc(-n2c3ccccc3c3cc(-c4ccc(N(c5ccc(-c6ccc7ccccc7c6)cc5)c5cccc(-c6cccc7c6ccc6ccccc67)c5)cc4)ccc32)cc1 ; c1cc(-c2cccc3c2ccc2ccccc23)cc(N(c2ccc(-c3ccc4ccccc4c3)cc2)c2cccc(-n3c4ccccc4c4ccccc43)c2)c1 ; c1ccc(-n2c3ccccc3c3ccc(-c4cccc(N(c5ccc(-c6ccc7ccccc7c6)cc5)c5cccc(-c6cccc7c6ccc6ccccc67)c5)c4)cc32)cc1 ; c1cc(-c2cccc3c2ccc2ccccc23)cc(N(c2ccc(-c3ccc4ccccc4c3)cc2)c2ccc(-n3c4ccccc4c4ccccc43)cc2)c1]

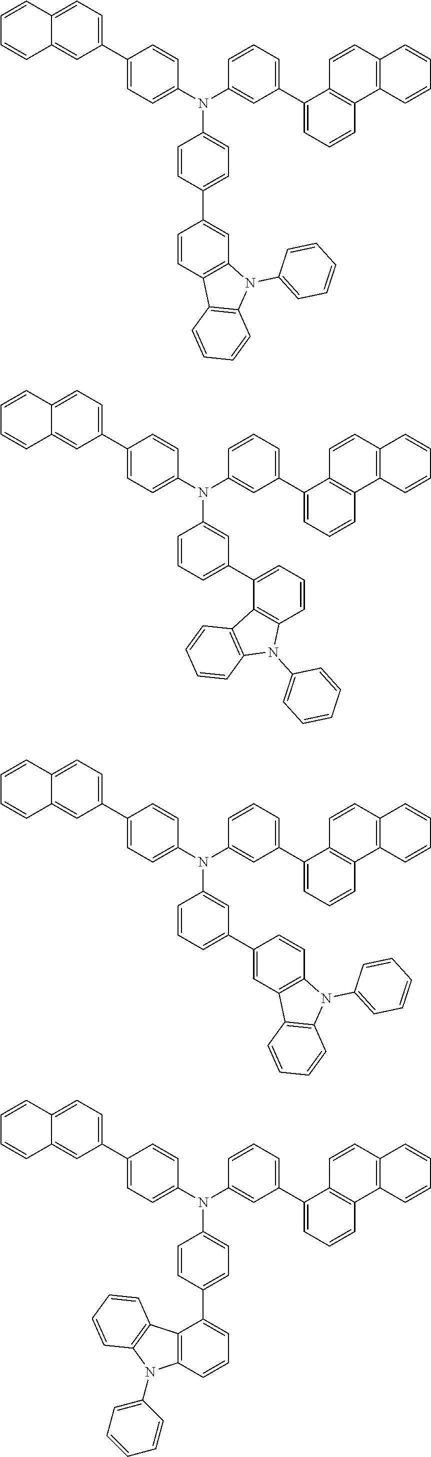 [SMILES: c1ccc(-n2c3ccccc3c3ccc(-c4ccc(N(c5ccc(-c6ccc7ccccc7c6)cc5)c5cccc(-c6cccc7c6ccc6ccccc67)c5)cc4)cc32)cc1 ; c1ccc(-n2c3ccccc3c3c(-c4cccc(N(c5ccc(-c6ccc7ccccc7c6)cc5)c5cccc(-c6cccc7c6ccc6ccccc67)c5)c4)cccc32)cc1 ; c1ccc(-n2c3ccccc3c3cc(-c4cccc(N(c5ccc(-c6ccc7ccccc7c6)cc5)c5cccc(-c6cccc7c6ccc6ccccc67)c5)c4)ccc32)cc1 ; c1ccc(-n2c3ccccc3c3c(-c4ccc(N(c5ccc(-c6ccc7ccccc7c6)cc5)c5cccc(-c6cccc7c6ccc6ccccc67)c5)cc4)cccc32)cc1]

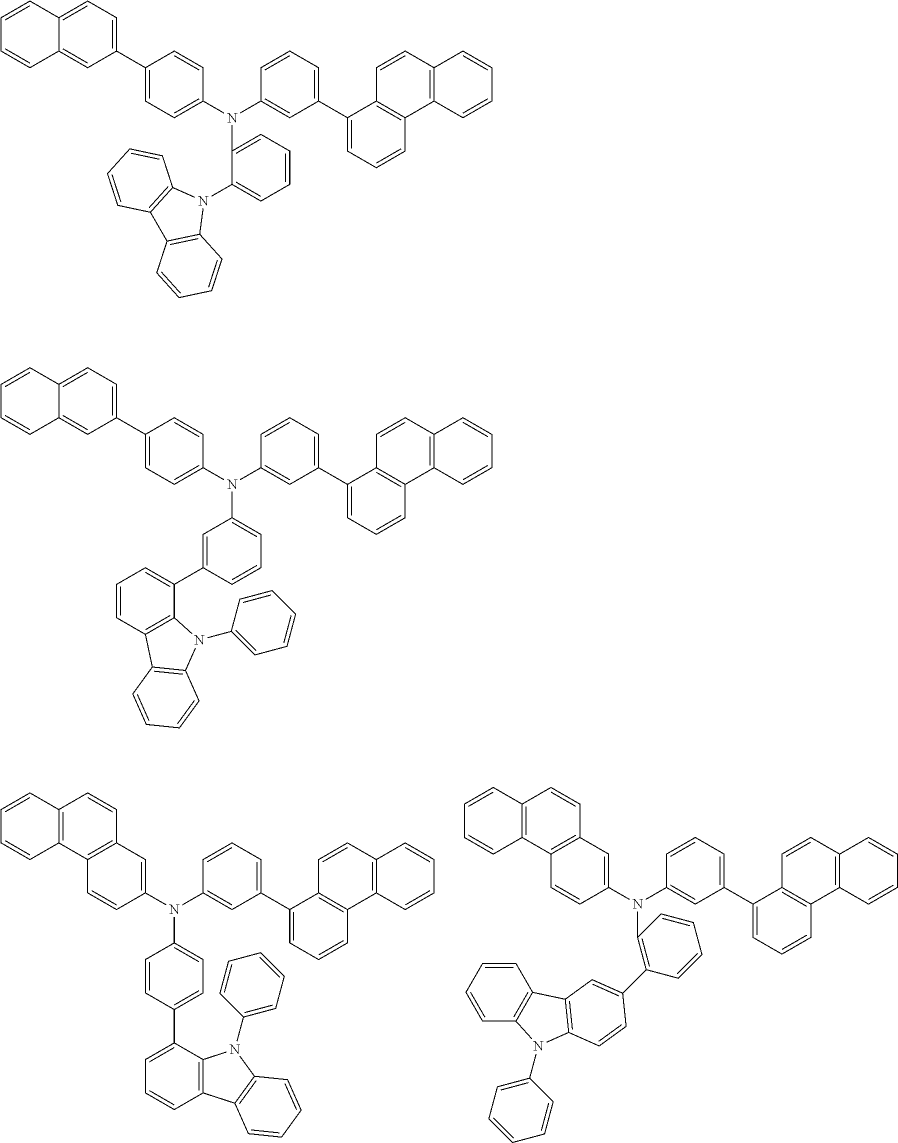

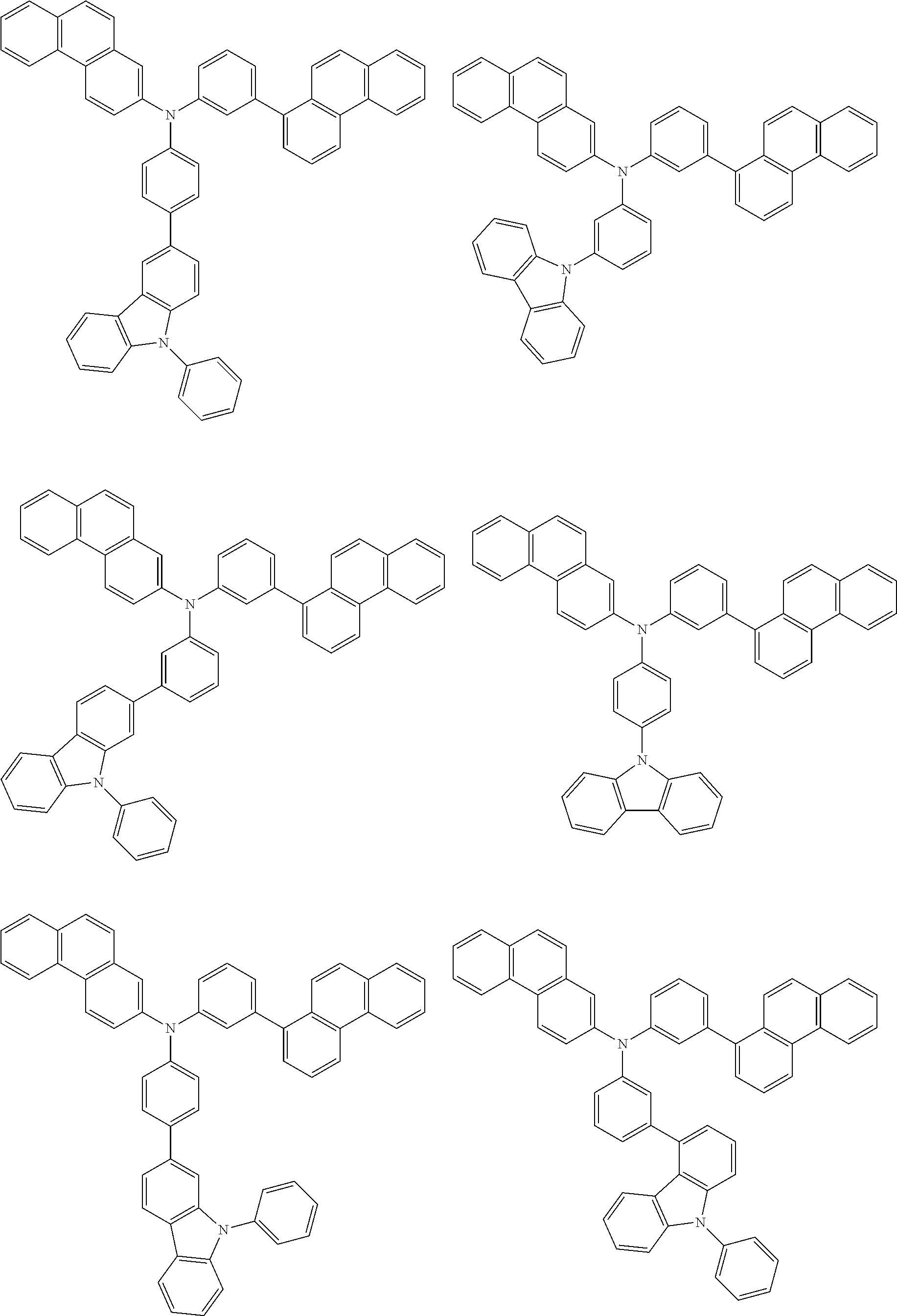

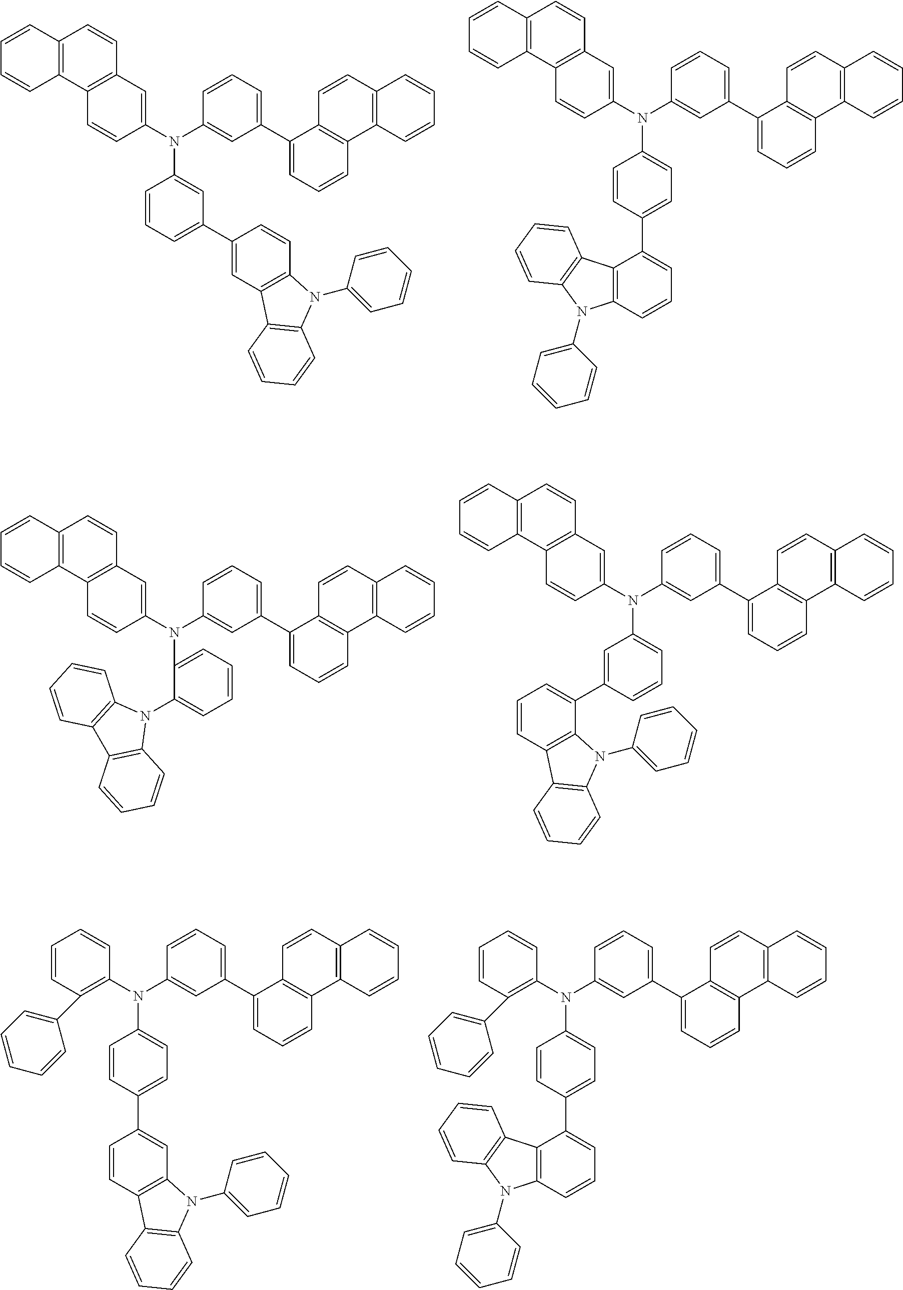

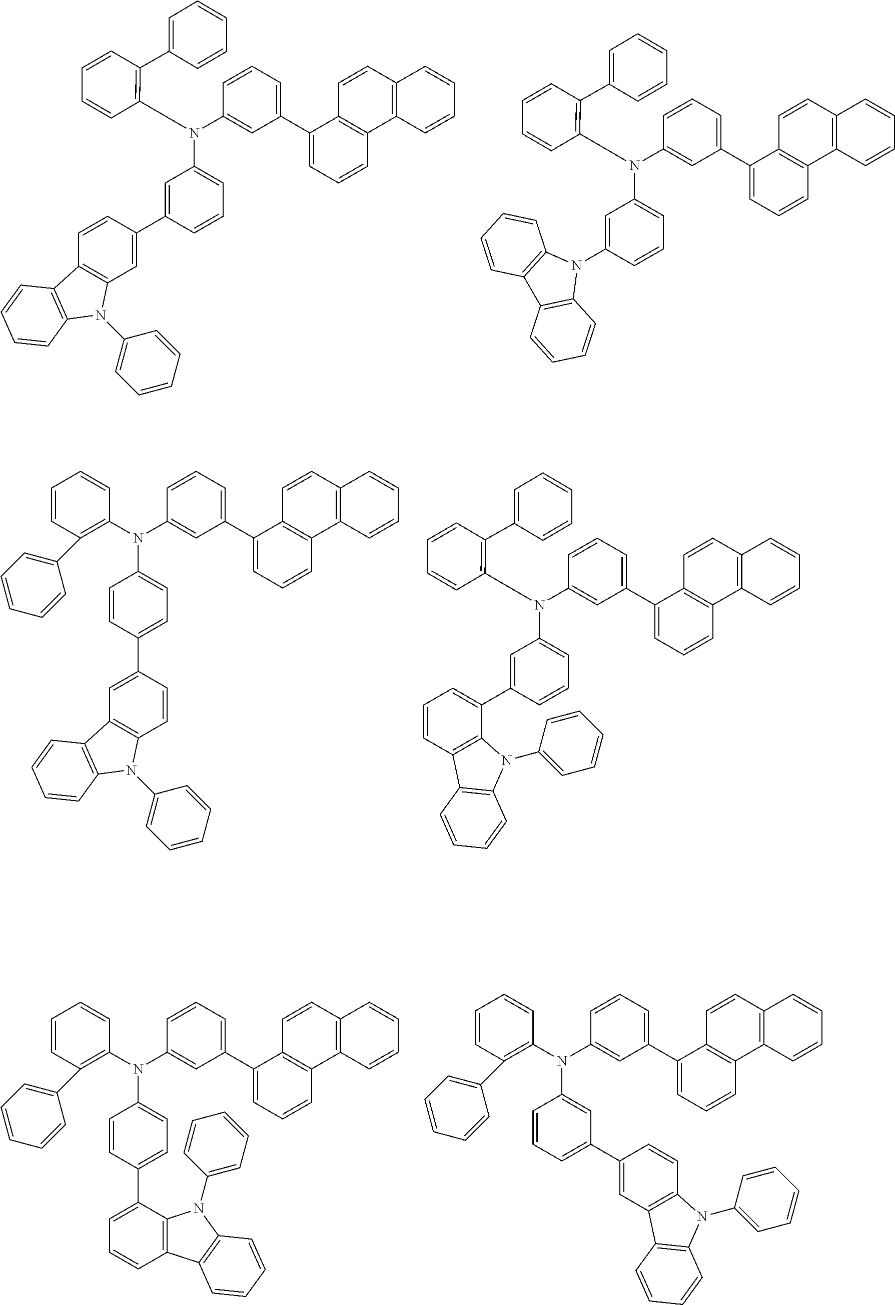

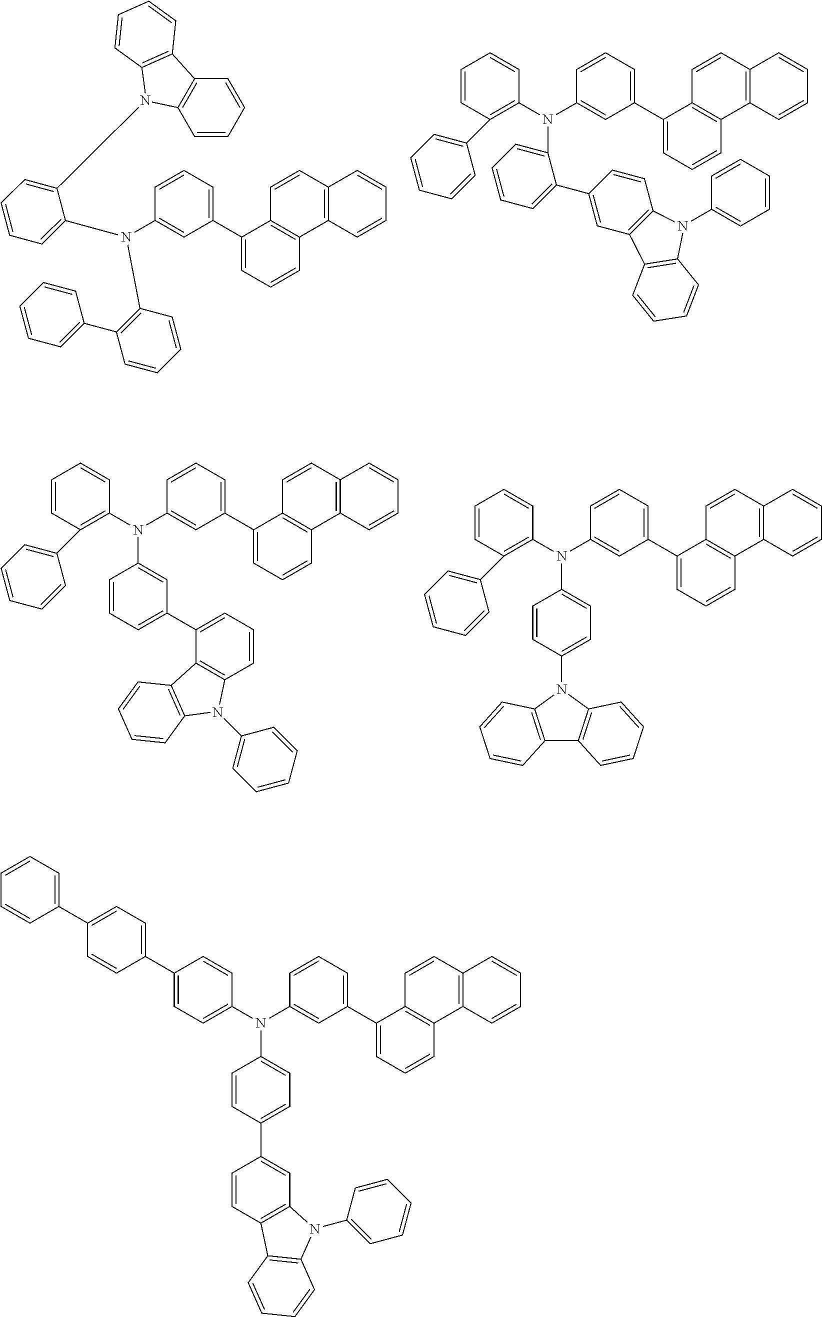

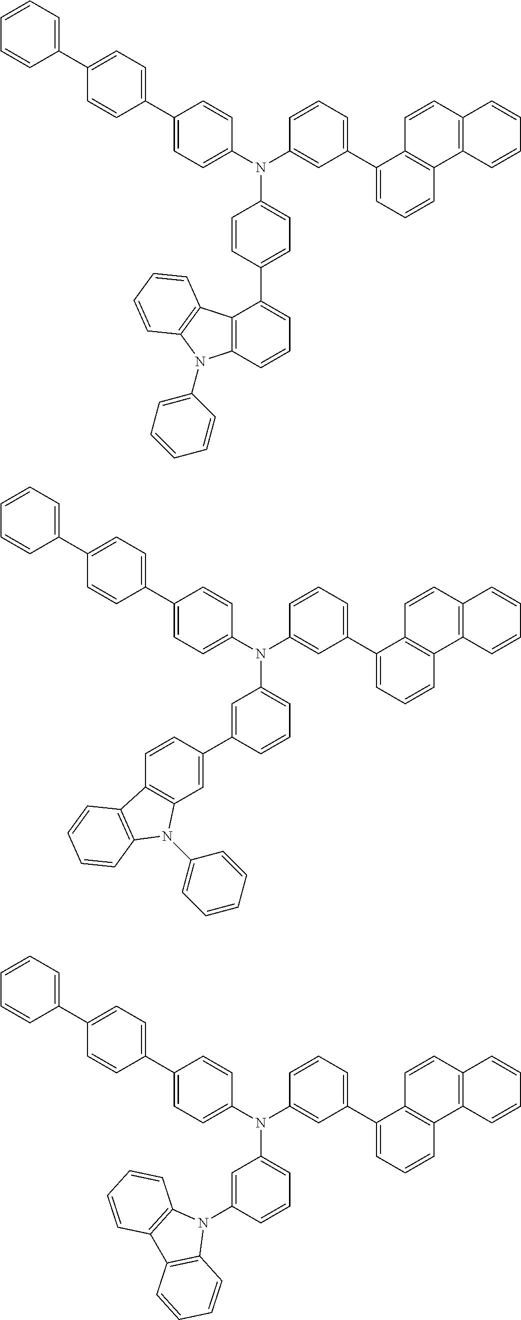 [SMILES: c1ccc(-c2ccc(-c3ccc(N(c4ccc(-c5cccc6c5c5ccccc5n6-c5ccccc5)cc4)c4cccc(-c5cccc6c5ccc5ccccc56)c4)cc3)cc2)cc1 ; c1ccc(-c2ccc(-c3ccc(N(c4cccc(-c5ccc6c7ccccc7n(-c7ccccc7)c6c5)c4)c4cccc(-c5cccc6c5ccc5ccccc56)c4)cc3)cc2)cc1 ; c1ccc(-c2ccc(-c3ccc(N(c4cccc(-c5cccc6c5ccc5ccccc56)c4)c4cccc(-n5c6ccccc6c6ccccc65)c4)cc3)cc2)cc1]

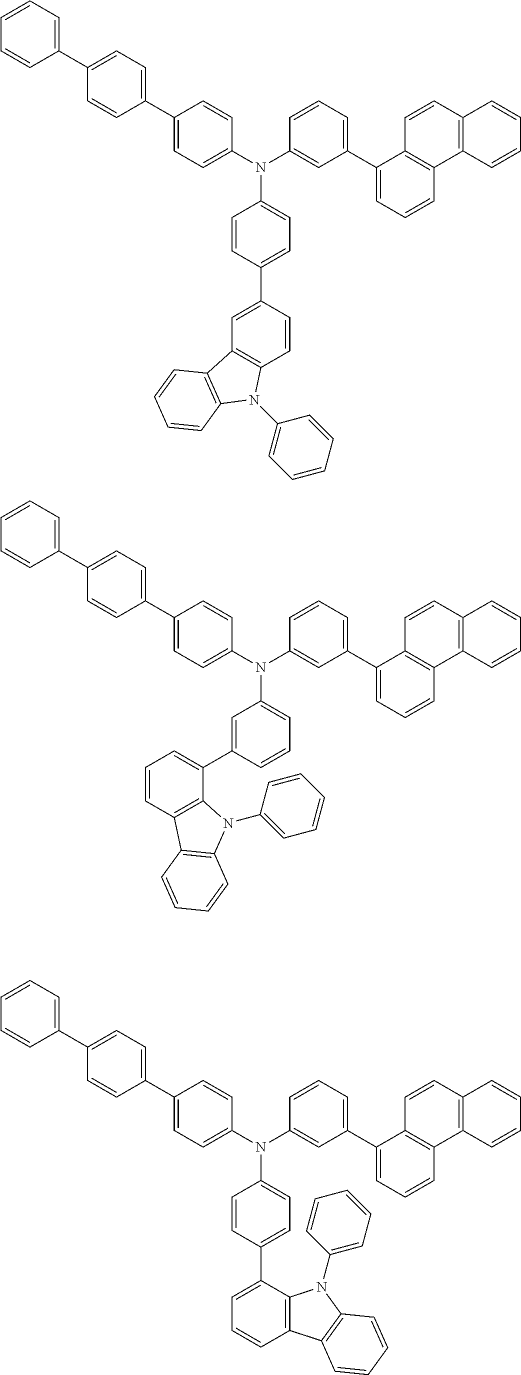 [SMILES: c1ccc(-c2ccc(-c3ccc(N(c4ccc(-c5ccc6c(c5)c5ccccc5n6-c5ccccc5)cc4)c4cccc(-c5cccc6c5ccc5ccccc56)c4)cc3)cc2)cc1 ; c1ccc(-c2ccc(-c3ccc(N(c4cccc(-c5cccc6c5ccc5ccccc56)c4)c4cccc(-c5cccc6c7ccccc7n(-c7ccccc7)c56)c4)cc3)cc2)cc1 ; c1ccc(-c2ccc(-c3ccc(N(c4ccc(-c5cccc6c7ccccc7n(-c7ccccc7)c56)cc4)c4cccc(-c5cccc6c5ccc5ccccc56)c4)cc3)cc2)cc1]

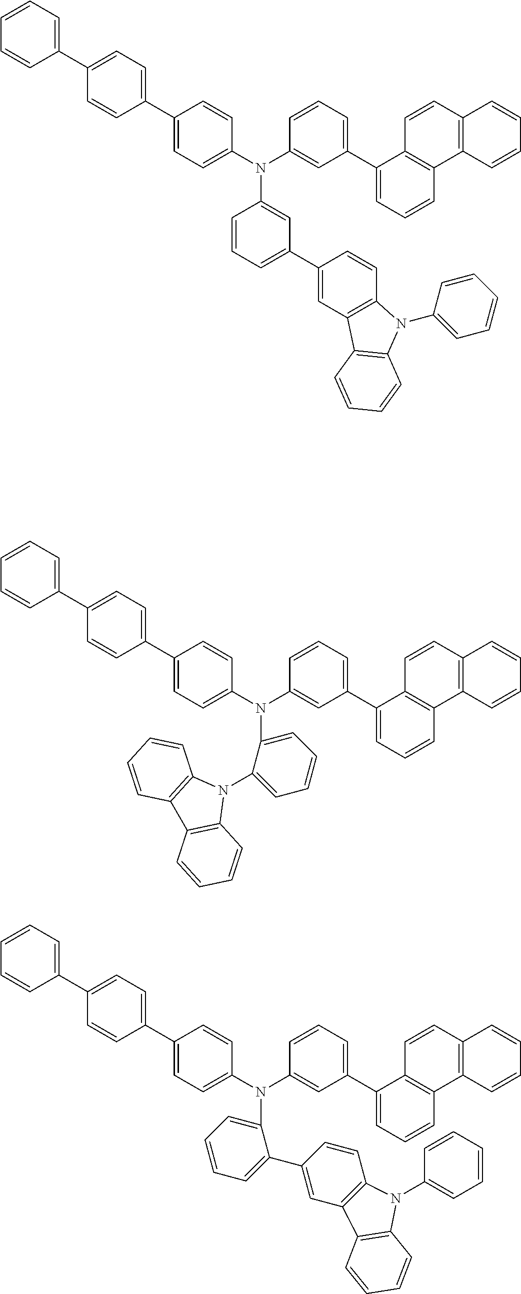 [SMILES: c1ccc(-c2ccc(-c3ccc(N(c4cccc(-c5ccc6c(c5)c5ccccc5n6-c5ccccc5)c4)c4cccc(-c5cccc6c5ccc5ccccc56)c4)cc3)cc2)cc1 ; c1ccc(-c2ccc(-c3ccc(N(c4cccc(-c5cccc6c5ccc5ccccc56)c4)c4ccccc4-n4c5ccccc5c5ccccc54)cc3)cc2)cc1 ; c1ccc(-c2ccc(-c3ccc(N(c4cccc(-c5cccc6c5ccc5ccccc56)c4)c4ccccc4-c4ccc5c(c4)c4ccccc4n5-c4ccccc4)cc3)cc2)cc1]

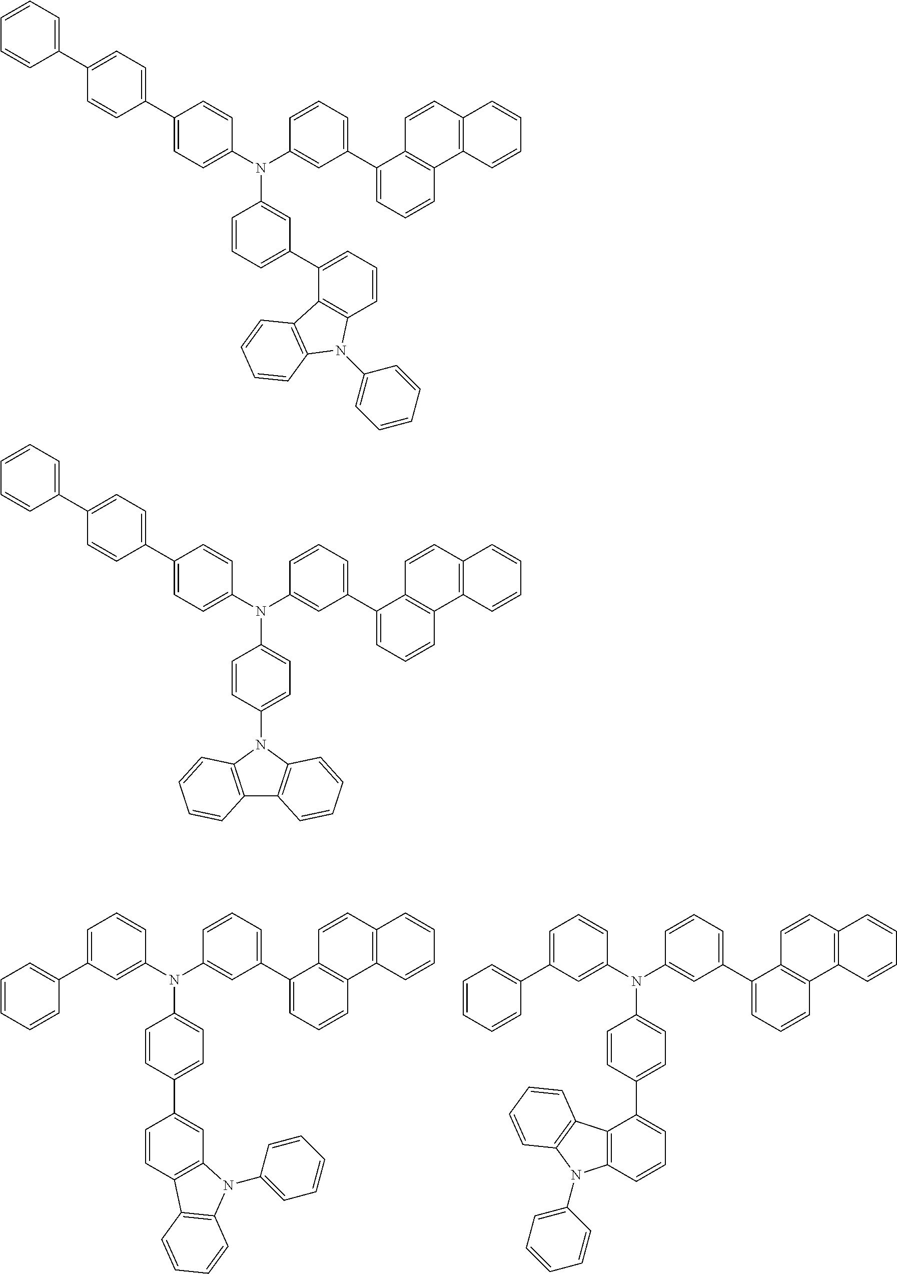

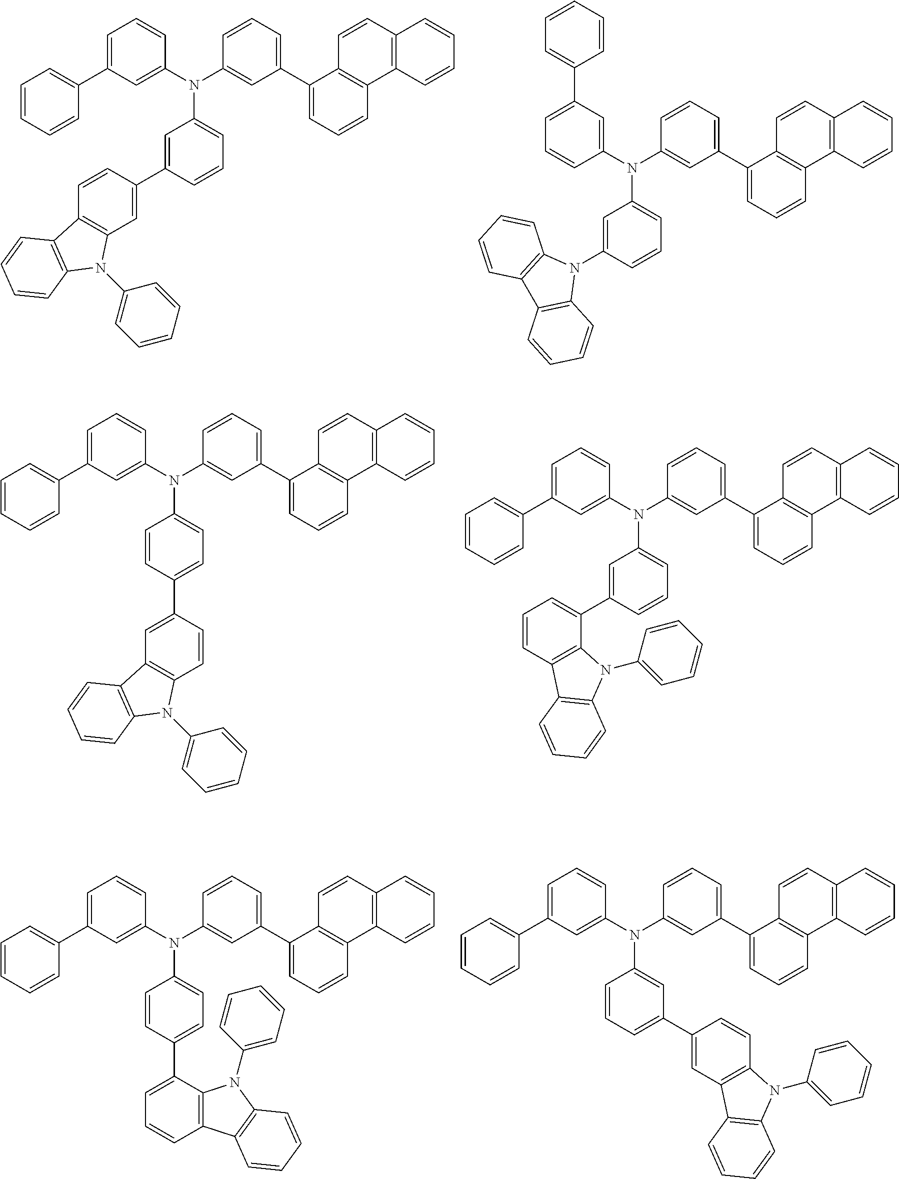

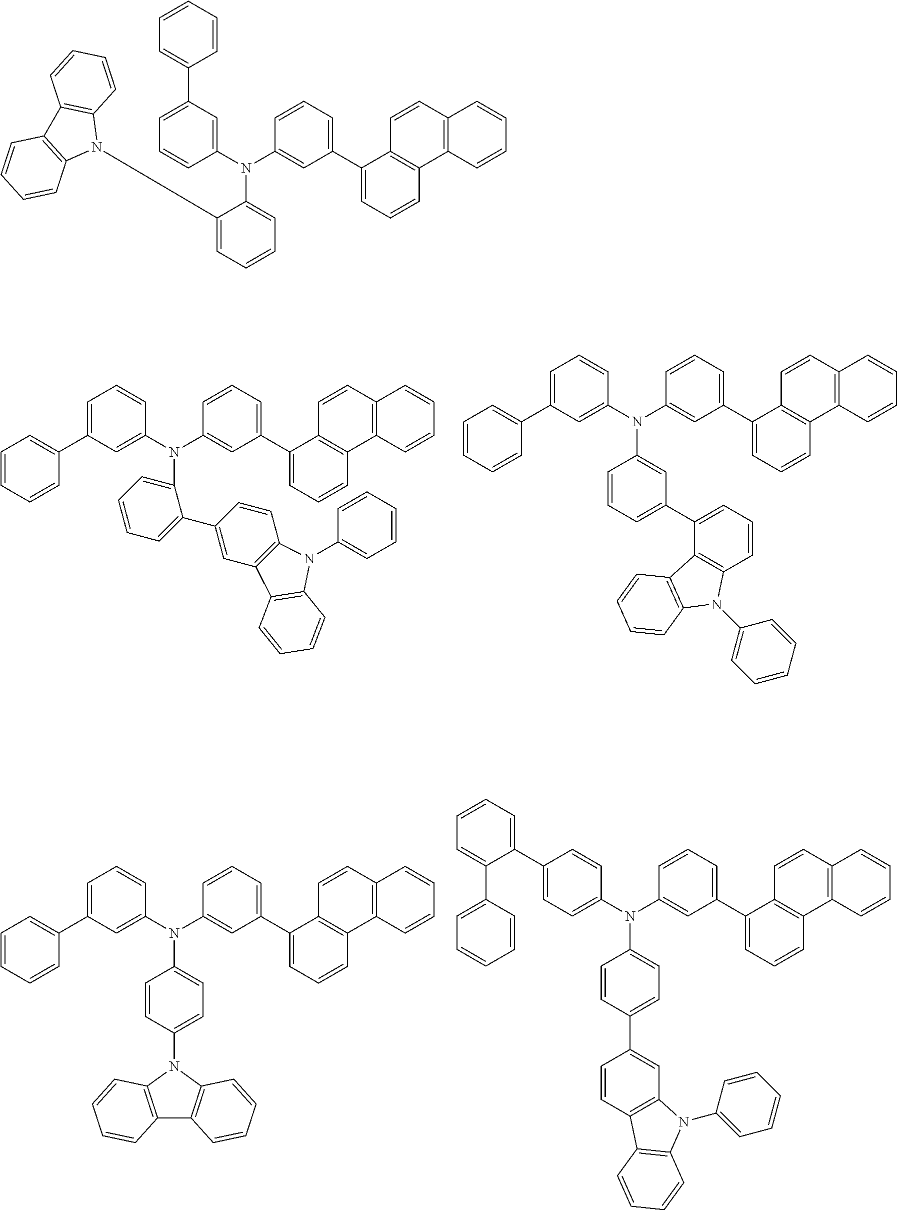

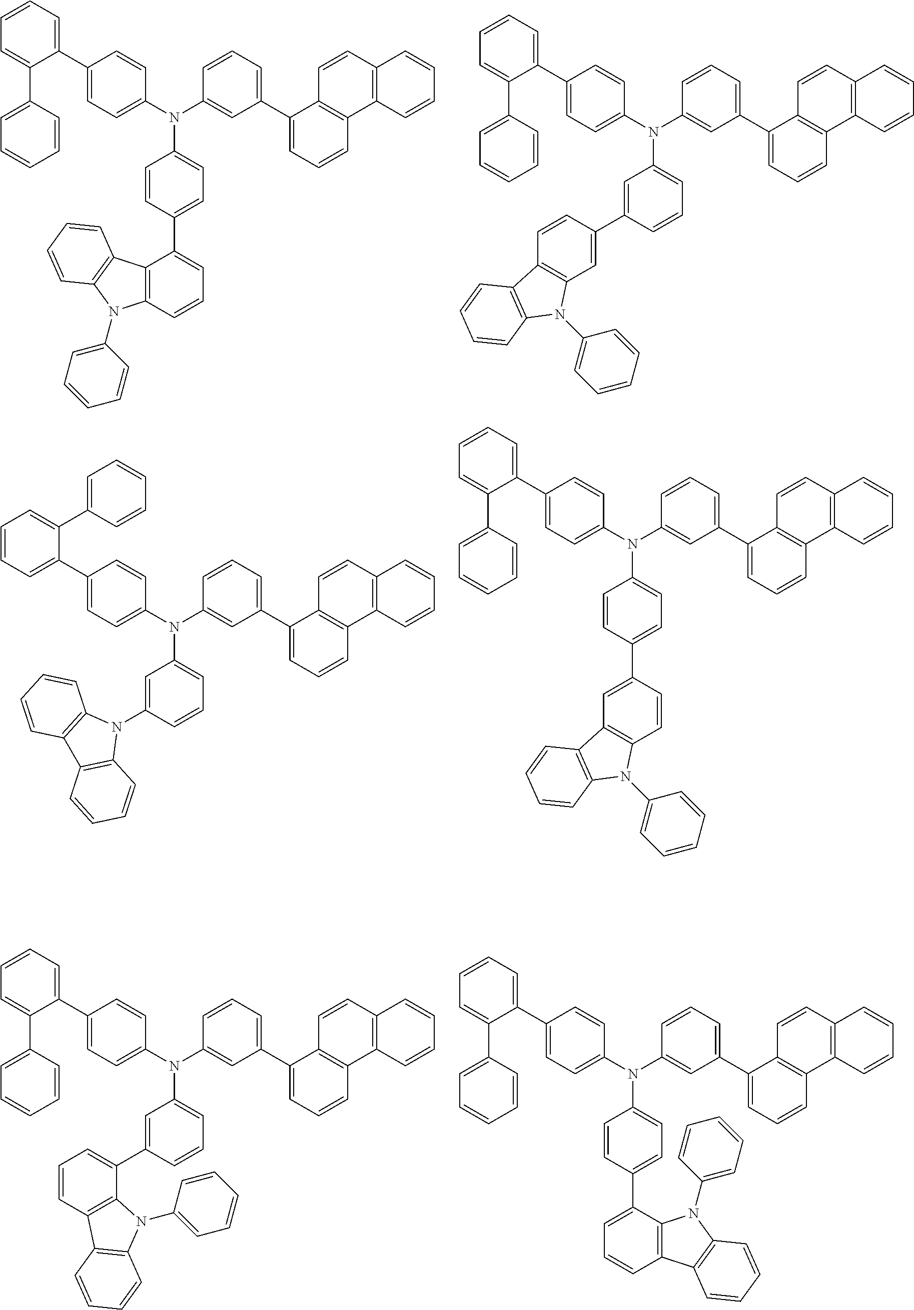

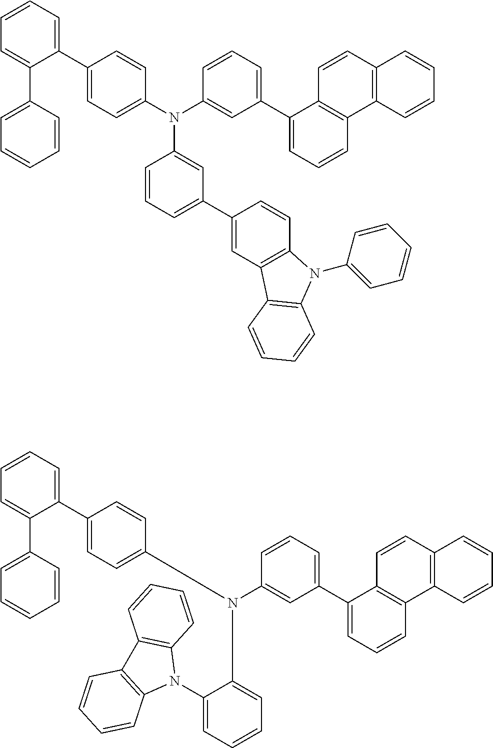 [SMILES: c1ccc(-c2ccccc2-c2ccc(N(c3cccc(-c4ccc5c(c4)c4ccccc4n5-c4ccccc4)c3)c3cccc(-c4cccc5c4ccc4ccccc45)c3)cc2)cc1 ; c1ccc(-c2ccccc2-c2ccc(N(c3cccc(-c4cccc5c4ccc4ccccc45)c3)c3ccccc3-n3c4ccccc4c4ccccc43)cc2)cc1]

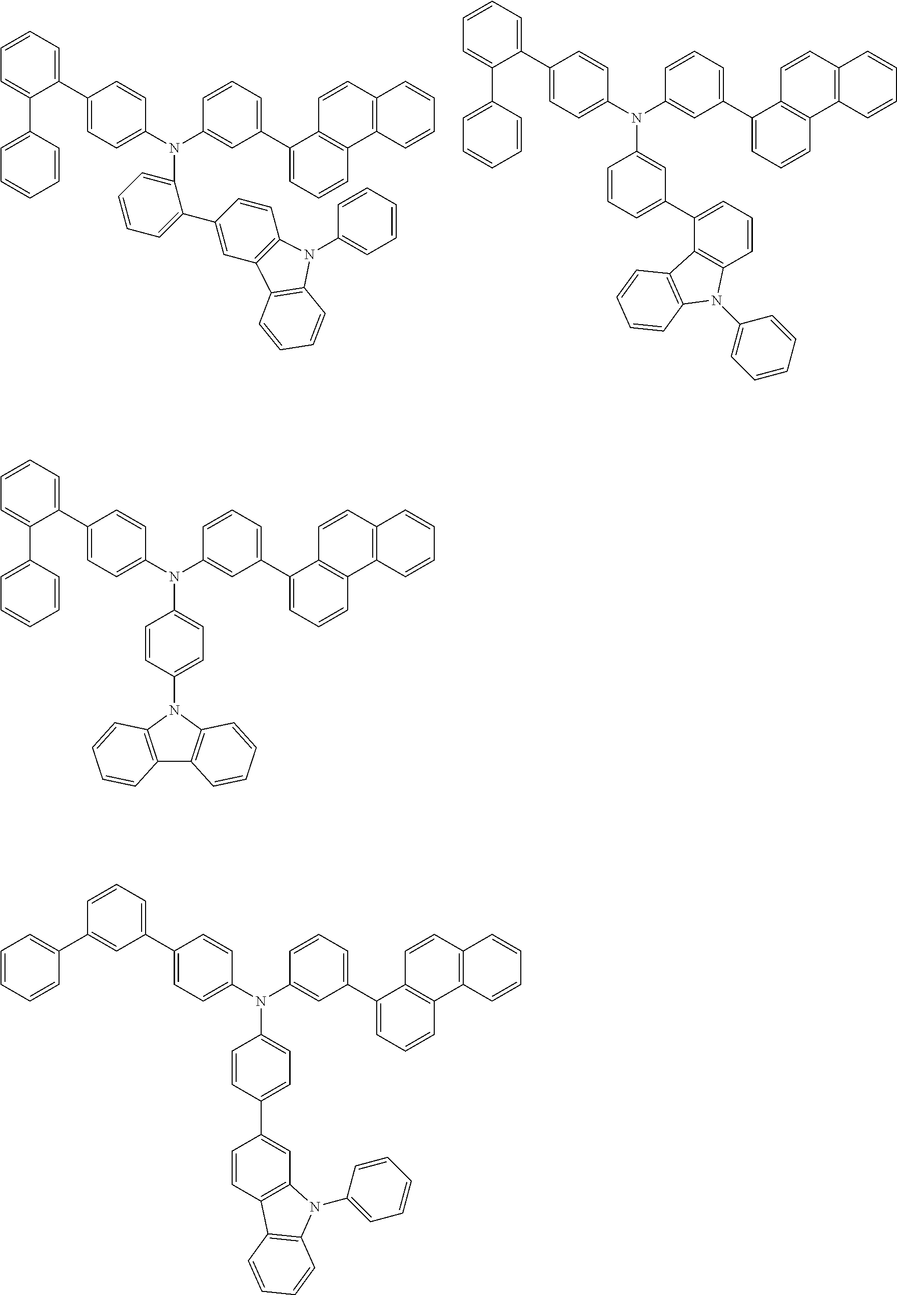

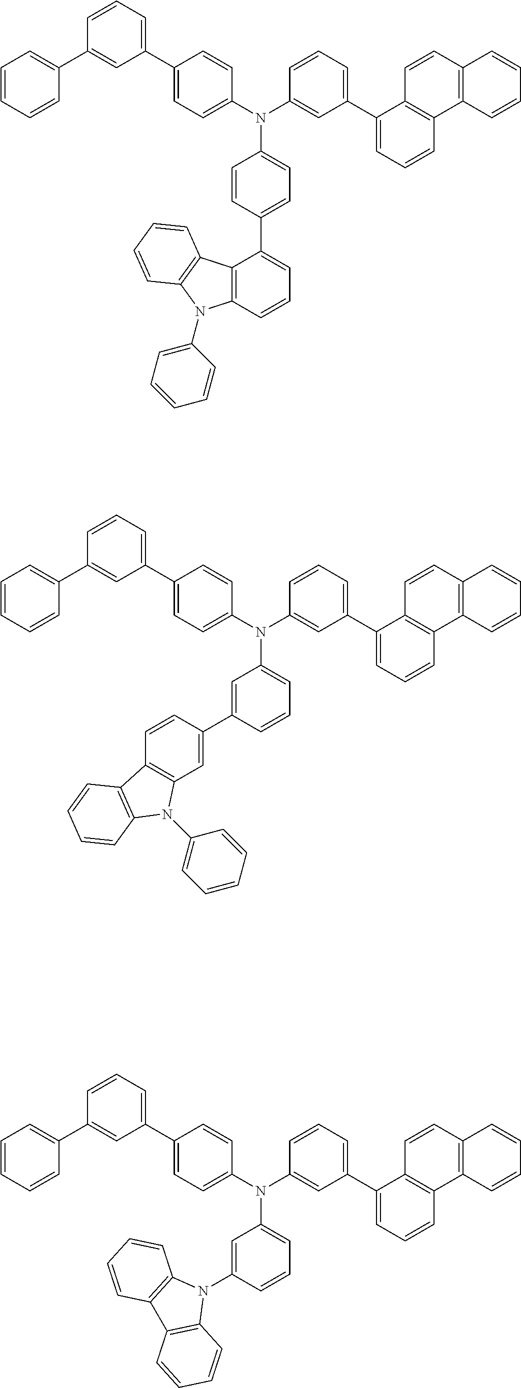 [SMILES: c1ccc(-c2cccc(-c3ccc(N(c4ccc(-c5cccc6c5c5ccccc5n6-c5ccccc5)cc4)c4cccc(-c5cccc6c5ccc5ccccc56)c4)cc3)c2)cc1 ; c1ccc(-c2cccc(-c3ccc(N(c4cccc(-c5ccc6c7ccccc7n(-c7ccccc7)c6c5)c4)c4cccc(-c5cccc6c5ccc5ccccc56)c4)cc3)c2)cc1 ; c1ccc(-c2cccc(-c3ccc(N(c4cccc(-c5cccc6c5ccc5ccccc56)c4)c4cccc(-n5c6ccccc6c6ccccc65)c4)cc3)c2)cc1]

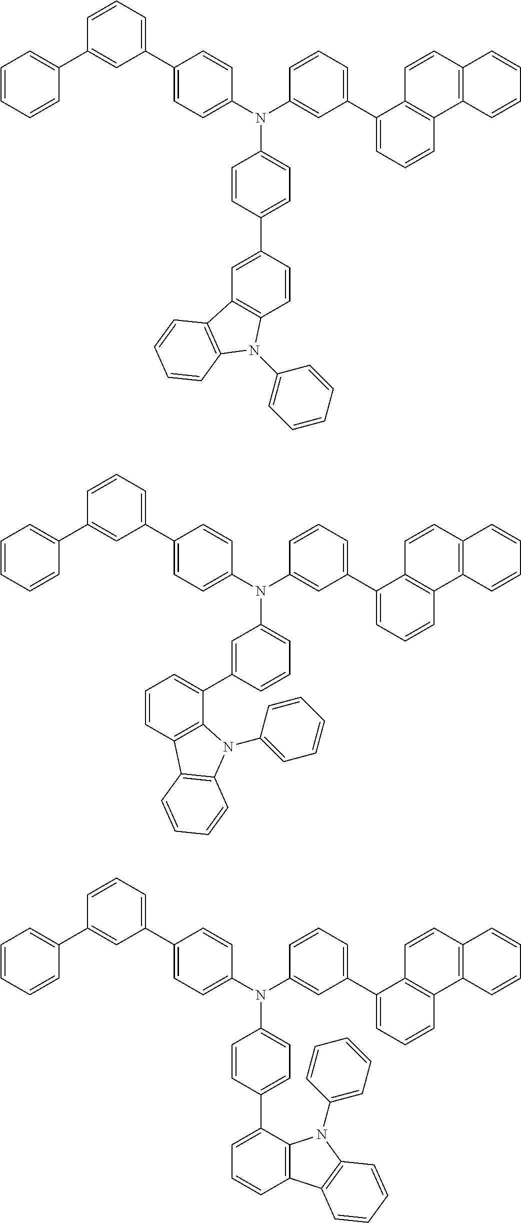 [SMILES: c1ccc(-c2cccc(-c3ccc(N(c4ccc(-c5ccc6c(c5)c5ccccc5n6-c5ccccc5)cc4)c4cccc(-c5cccc6c5ccc5ccccc56)c4)cc3)c2)cc1 ; c1ccc(-c2cccc(-c3ccc(N(c4cccc(-c5cccc6c5ccc5ccccc56)c4)c4cccc(-c5cccc6c7ccccc7n(-c7ccccc7)c56)c4)cc3)c2)cc1 ; c1ccc(-c2cccc(-c3ccc(N(c4ccc(-c5cccc6c7ccccc7n(-c7ccccc7)c56)cc4)c4cccc(-c5cccc6c5ccc5ccccc56)c4)cc3)c2)cc1]

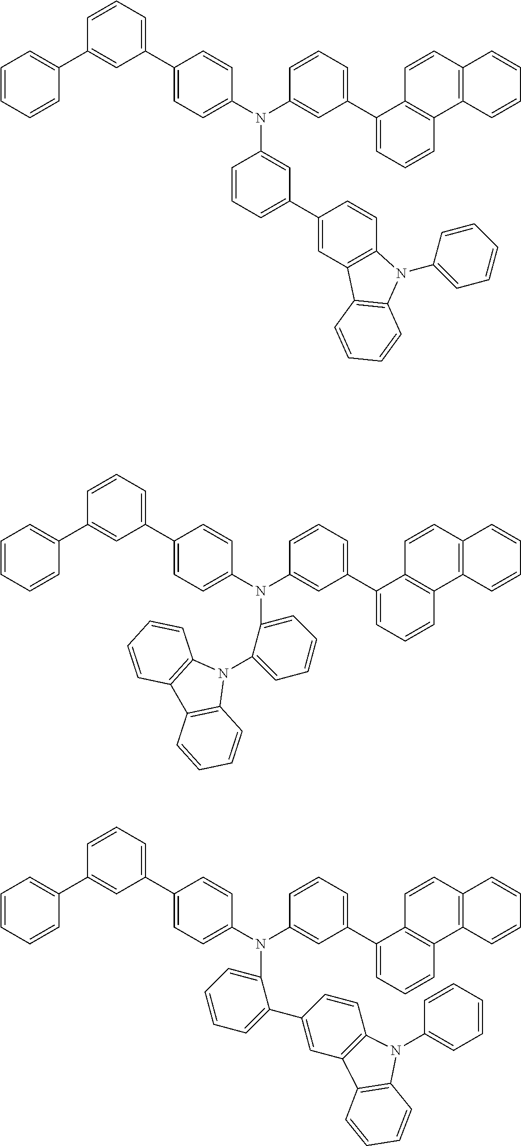 [SMILES: c1ccc(-c2cccc(-c3ccc(N(c4cccc(-c5ccc6c(c5)c5ccccc5n6-c5ccccc5)c4)c4cccc(-c5cccc6c5ccc5ccccc56)c4)cc3)c2)cc1 ; c1ccc(-c2cccc(-c3ccc(N(c4cccc(-c5cccc6c5ccc5ccccc56)c4)c4ccccc4-n4c5ccccc5c5ccccc54)cc3)c2)cc1 ; c1ccc(-c2cccc(-c3ccc(N(c4cccc(-c5cccc6c5ccc5ccccc56)c4)c4ccccc4-c4ccc5c(c4)c4ccccc4n5-c4ccccc4)cc3)c2)cc1]

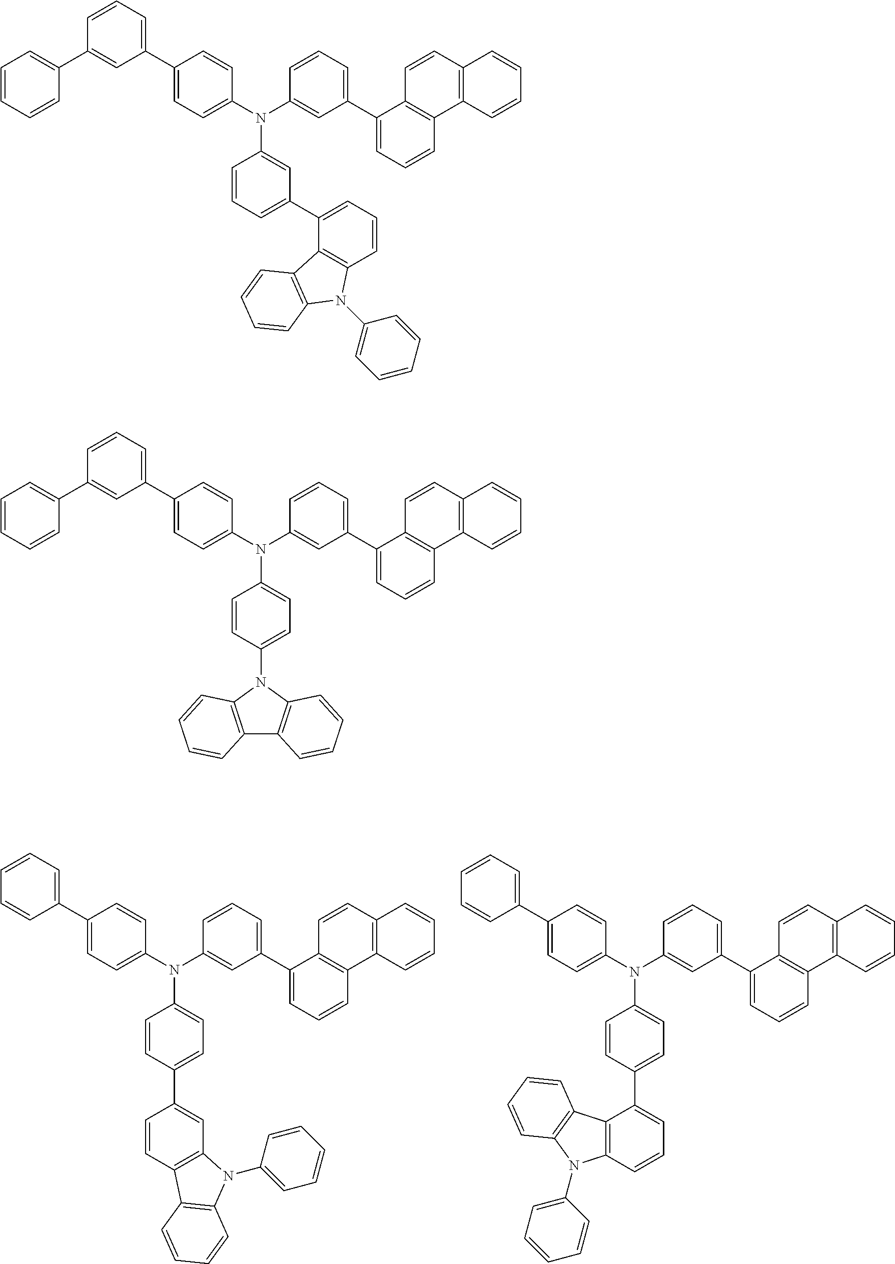

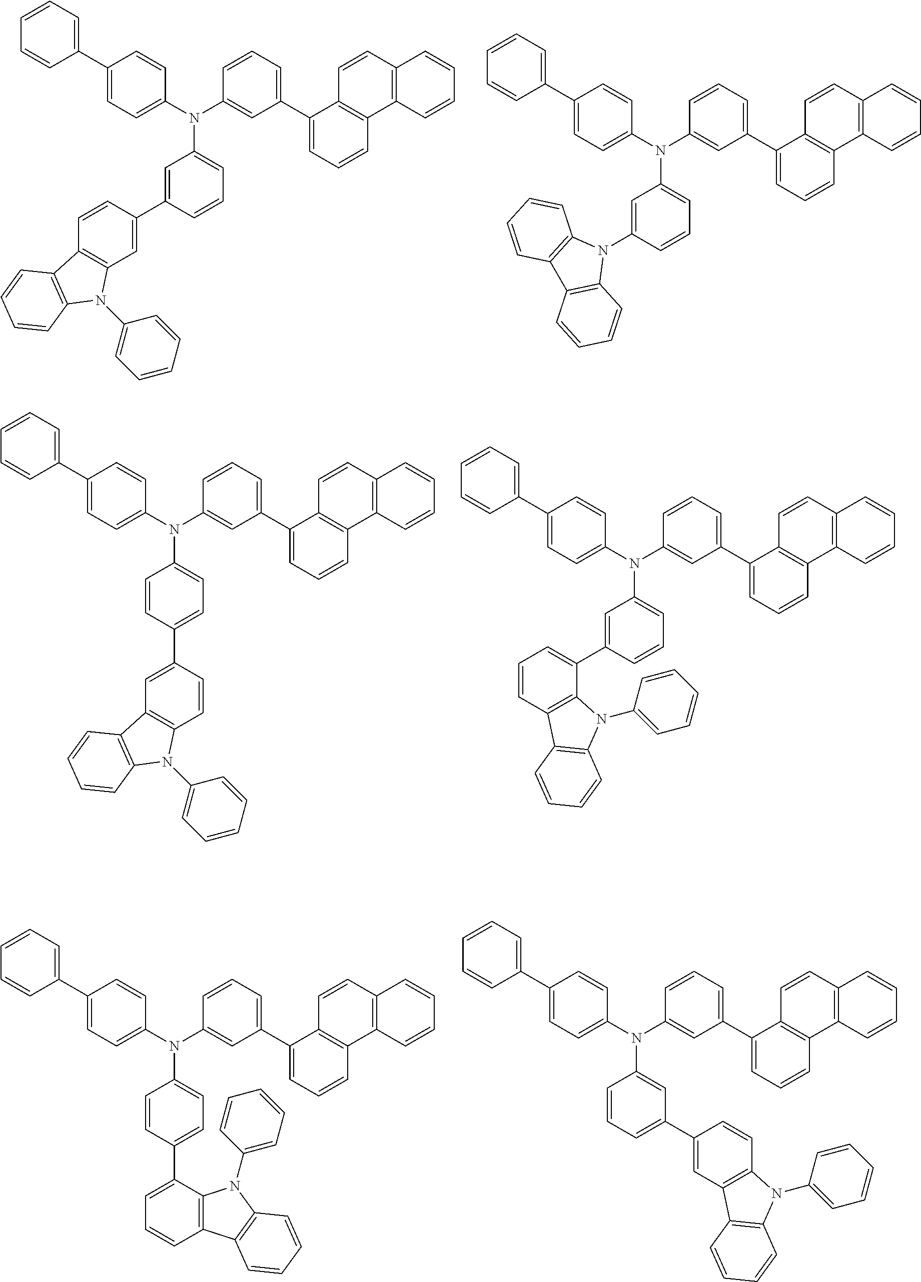

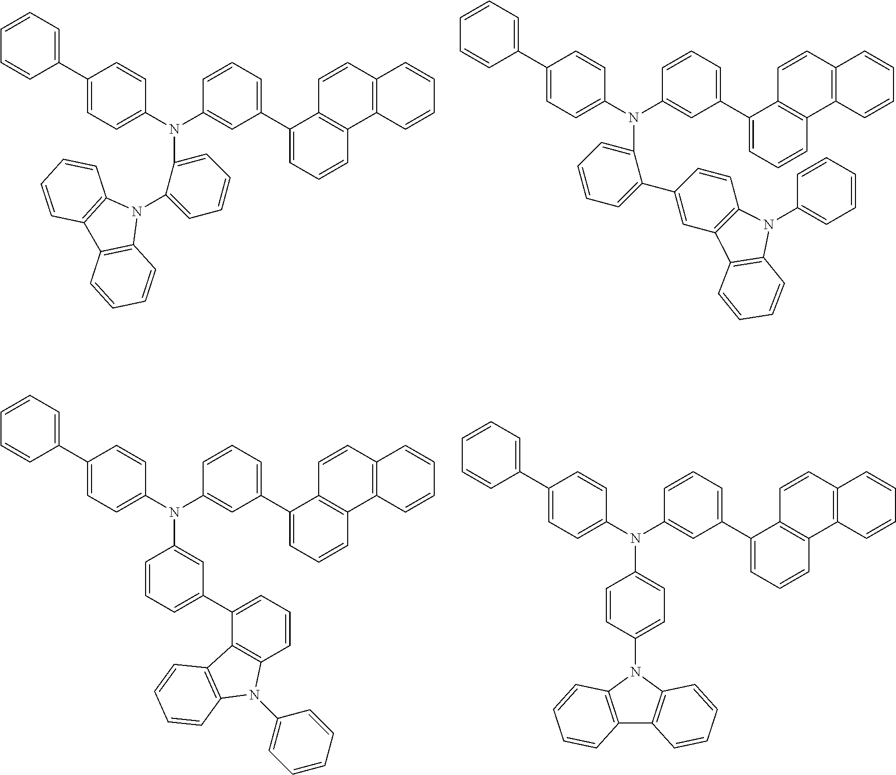

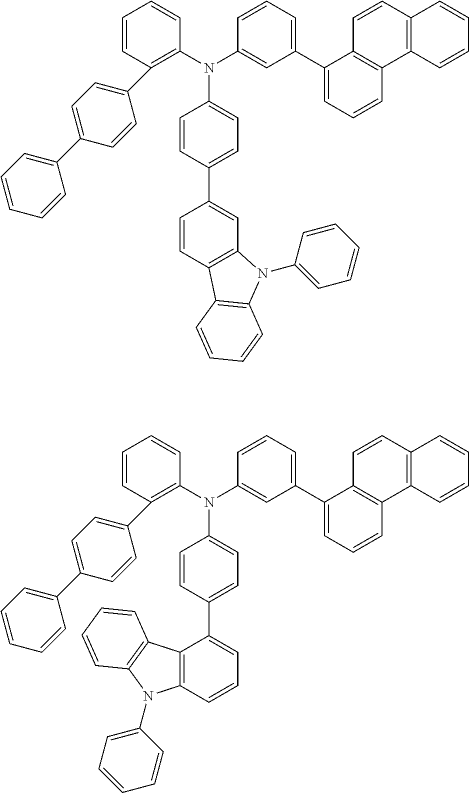 [SMILES: c1ccc(-c2ccc(-c3ccccc3N(c3ccc(-c4ccc5c6ccccc6n(-c6ccccc6)c5c4)cc3)c3cccc(-c4cccc5c4ccc4ccccc45)c3)cc2)cc1 ; c1ccc(-c2ccc(-c3ccccc3N(c3ccc(-c4cccc5c4c4ccccc4n5-c4ccccc4)cc3)c3cccc(-c4cccc5c4ccc4ccccc45)c3)cc2)cc1]

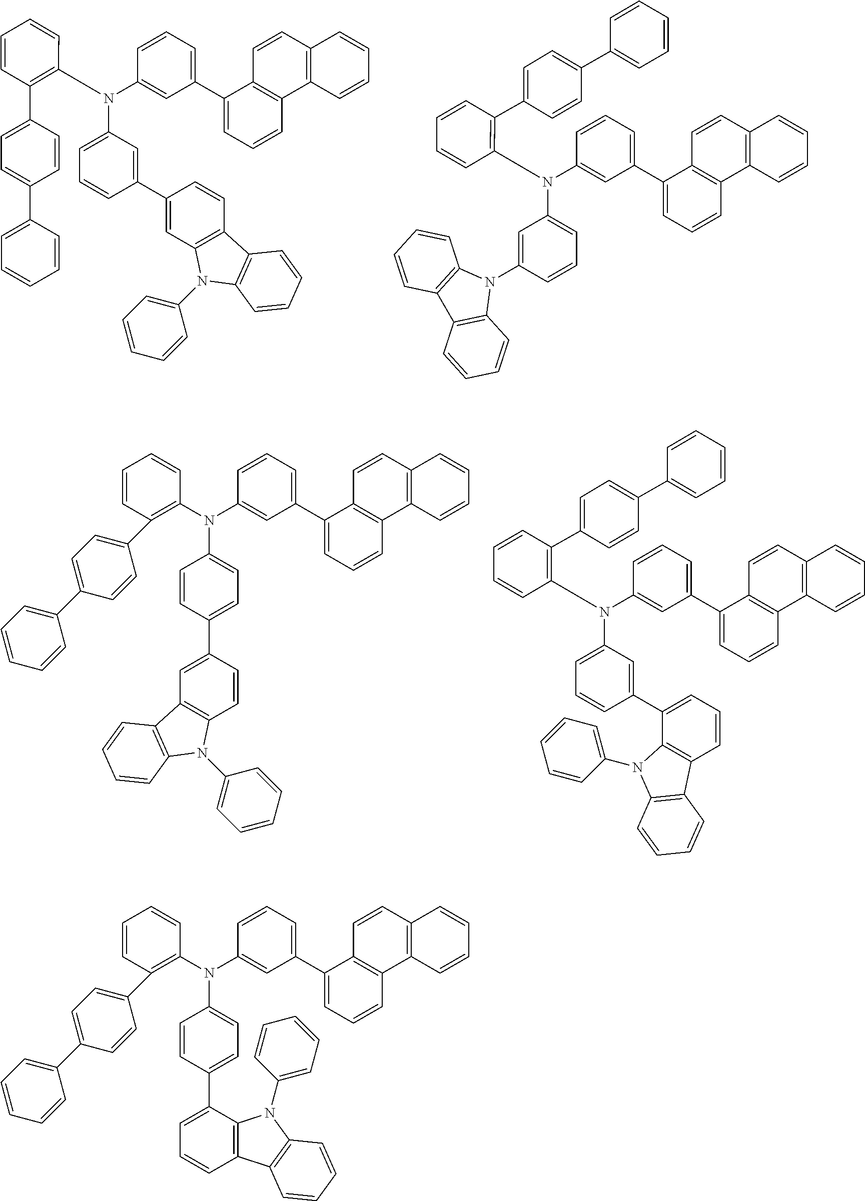

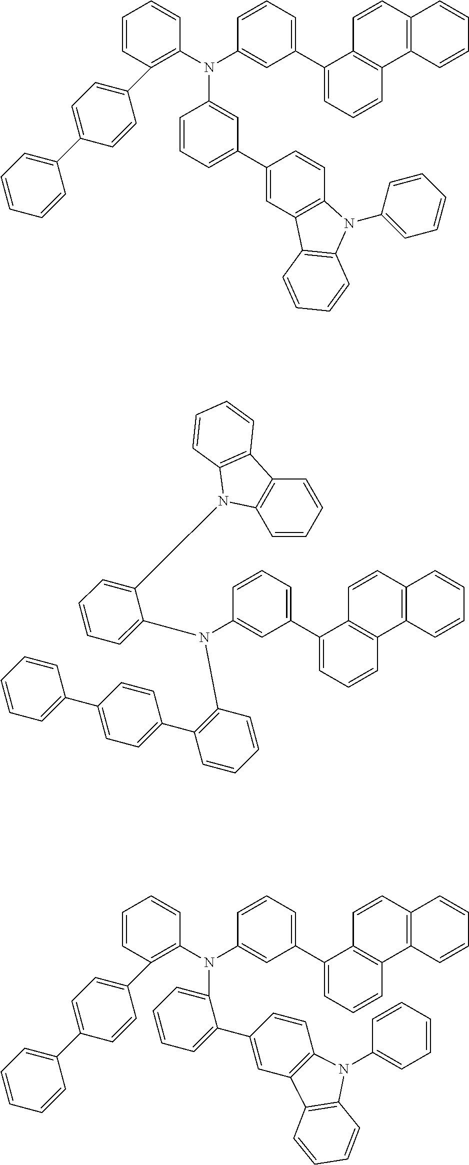 [SMILES: c1ccc(-c2ccc(-c3ccccc3N(c3cccc(-c4ccc5c(c4)c4ccccc4n5-c4ccccc4)c3)c3cccc(-c4cccc5c4ccc4ccccc45)c3)cc2)cc1 ; c1ccc(-c2ccc(-c3ccccc3N(c3cccc(-c4cccc5c4ccc4ccccc45)c3)c3ccccc3-n3c4ccccc4c4ccccc43)cc2)cc1 ; c1ccc(-c2ccc(-c3ccccc3N(c3cccc(-c4cccc5c4ccc4ccccc45)c3)c3ccccc3-c3ccc4c(c3)c3ccccc3n4-c3ccccc3)cc2)cc1]

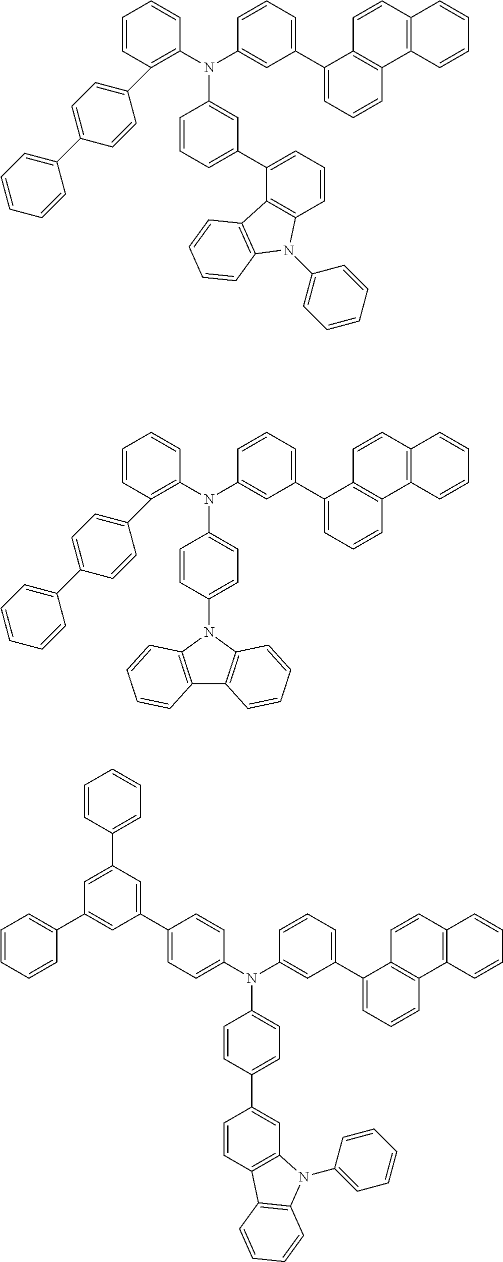 [SMILES: c1ccc(-c2ccc(-c3ccccc3N(c3cccc(-c4cccc5c4ccc4ccccc45)c3)c3cccc(-c4cccc5c4c4ccccc4n5-c4ccccc4)c3)cc2)cc1 ; c1ccc(-c2ccc(-c3ccccc3N(c3ccc(-n4c5ccccc5c5ccccc54)cc3)c3cccc(-c4cccc5c4ccc4ccccc45)c3)cc2)cc1 ; c1ccc(-c2cc(-c3ccccc3)cc(-c3ccc(N(c4ccc(-c5ccc6c7ccccc7n(-c7ccccc7)c6c5)cc4)c4cccc(-c5cccc6c5ccc5ccccc56)c4)cc3)c2)cc1]

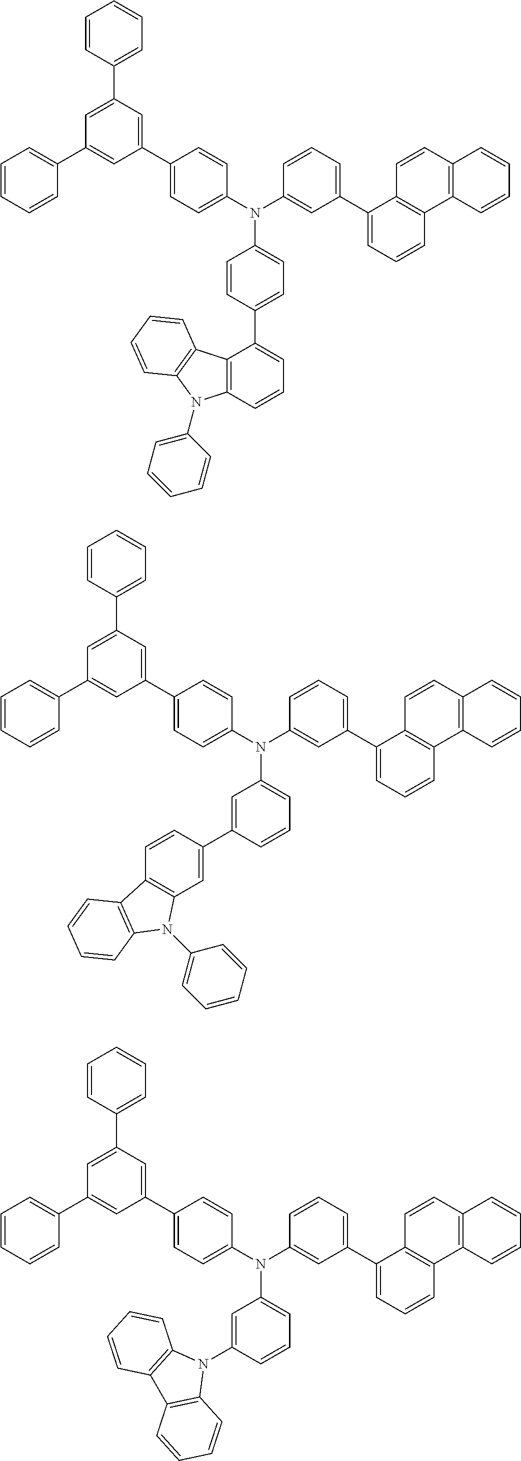 [SMILES: c1ccc(-c2cc(-c3ccccc3)cc(-c3ccc(N(c4ccc(-c5cccc6c5c5ccccc5n6-c5ccccc5)cc4)c4cccc(-c5cccc6c5ccc5ccccc56)c4)cc3)c2)cc1 ; c1ccc(-c2cc(-c3ccccc3)cc(-c3ccc(N(c4cccc(-c5ccc6c7ccccc7n(-c7ccccc7)c6c5)c4)c4cccc(-c5cccc6c5ccc5ccccc56)c4)cc3)c2)cc1 ; c1ccc(-c2cc(-c3ccccc3)cc(-c3ccc(N(c4cccc(-c5cccc6c5ccc5ccccc56)c4)c4cccc(-n5c6ccccc6c6ccccc65)c4)cc3)c2)cc1]

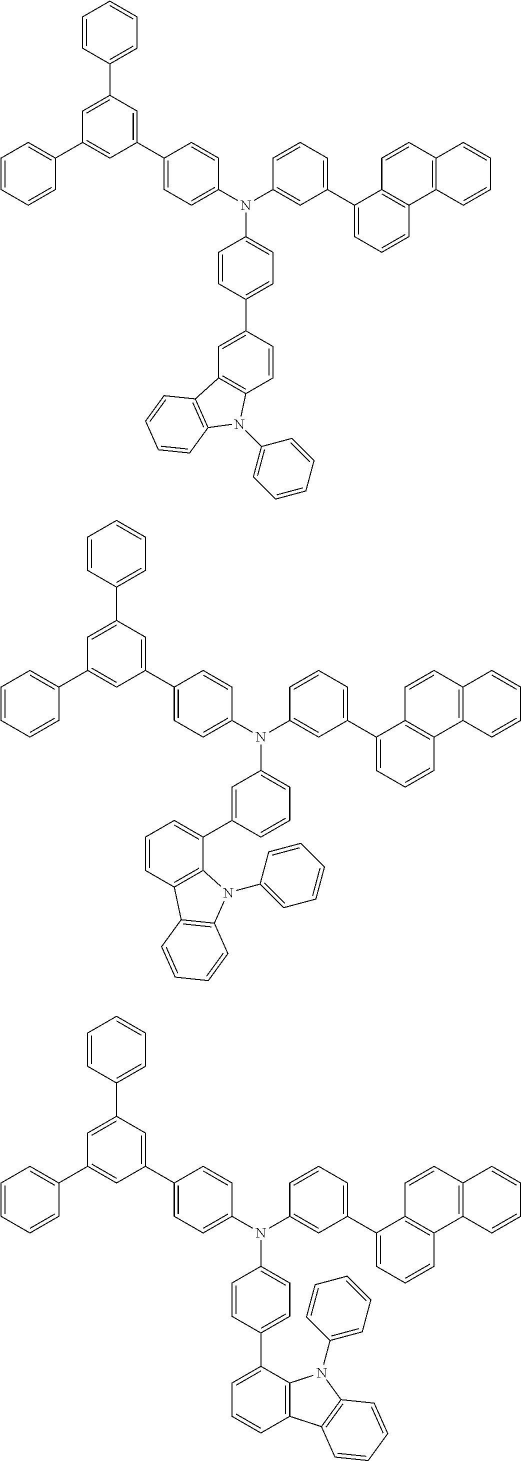 [SMILES: c1ccc(-c2cc(-c3ccccc3)cc(-c3ccc(N(c4ccc(-c5ccc6c(c5)c5ccccc5n6-c5ccccc5)cc4)c4cccc(-c5cccc6c5ccc5ccccc56)c4)cc3)c2)cc1 ; c1ccc(-c2cc(-c3ccccc3)cc(-c3ccc(N(c4cccc(-c5cccc6c5ccc5ccccc56)c4)c4cccc(-c5cccc6c7ccccc7n(-c7ccccc7)c56)c4)cc3)c2)cc1 ; c1ccc(-c2cc(-c3ccccc3)cc(-c3ccc(N(c4ccc(-c5cccc6c7ccccc7n(-c7ccccc7)c56)cc4)c4cccc(-c5cccc6c5ccc5ccccc56)c4)cc3)c2)cc1]

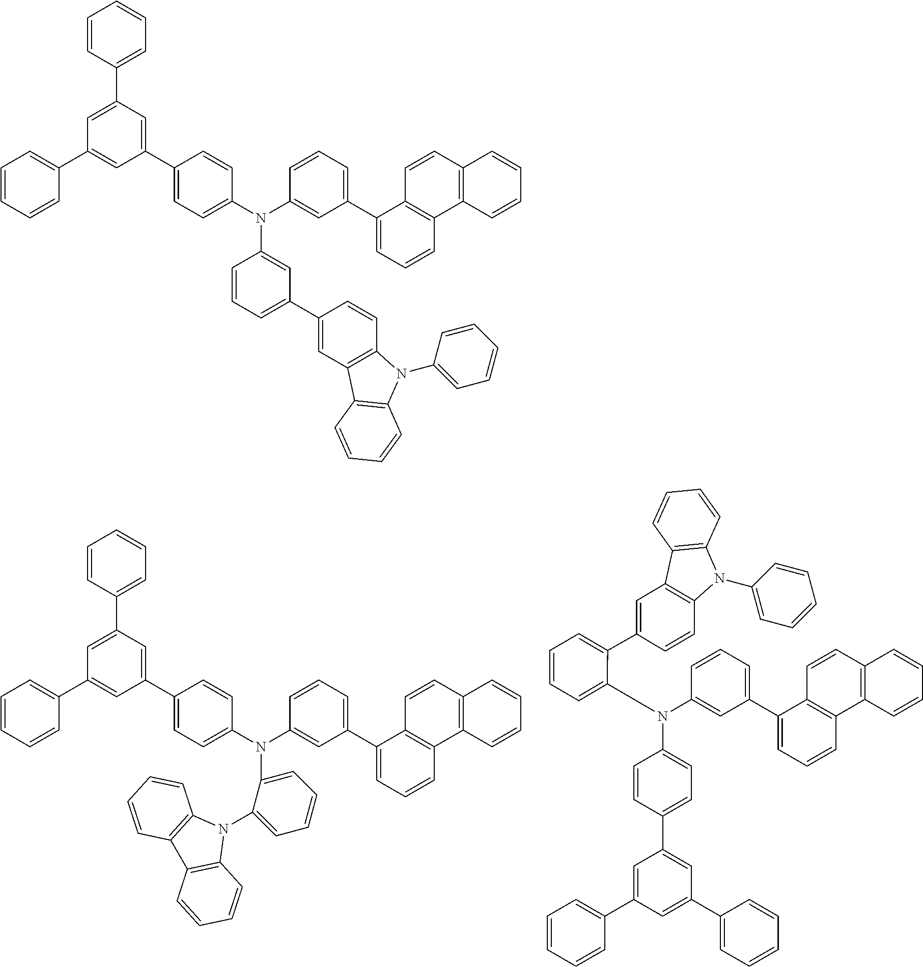

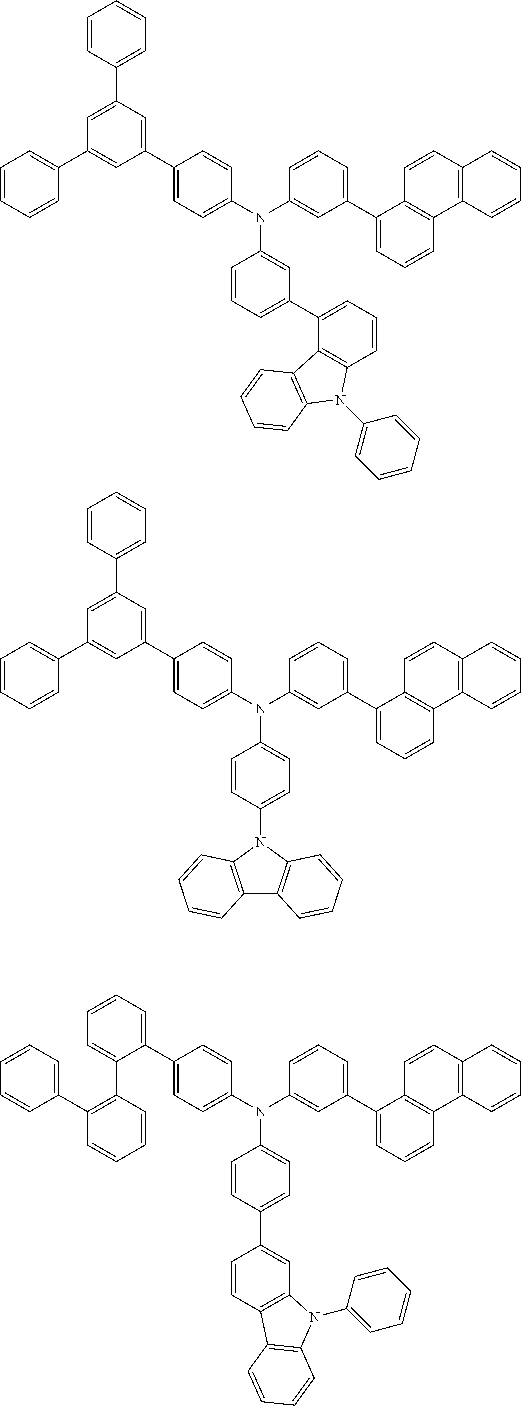 [SMILES: c1ccc(-c2cc(-c3ccccc3)cc(-c3ccc(N(c4cccc(-c5cccc6c5ccc5ccccc56)c4)c4cccc(-c5cccc6c5c5ccccc5n6-c5ccccc5)c4)cc3)c2)cc1 ; c1ccc(-c2cc(-c3ccccc3)cc(-c3ccc(N(c4ccc(-n5c6ccccc6c6ccccc65)cc4)c4cccc(-c5cccc6c5ccc5ccccc56)c4)cc3)c2)cc1 ; c1ccc(-c2ccccc2-c2ccccc2-c2ccc(N(c3ccc(-c4ccc5c6ccccc6n(-c6ccccc6)c5c4)cc3)c3cccc(-c4cccc5c4ccc4ccccc45)c3)cc2)cc1]

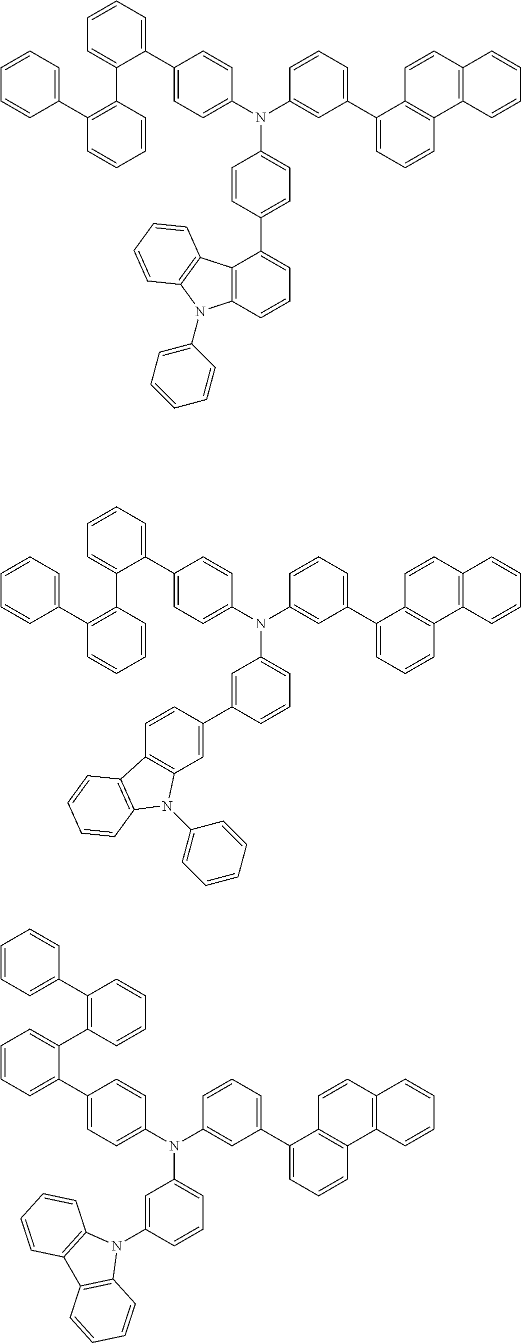 [SMILES: c1ccc(-c2ccccc2-c2ccccc2-c2ccc(N(c3ccc(-c4cccc5c4c4ccccc4n5-c4ccccc4)cc3)c3cccc(-c4cccc5c4ccc4ccccc45)c3)cc2)cc1 ; c1ccc(-c2ccccc2-c2ccccc2-c2ccc(N(c3cccc(-c4ccc5c6ccccc6n(-c6ccccc6)c5c4)c3)c3cccc(-c4cccc5c4ccc4ccccc45)c3)cc2)cc1 ; c1ccc(-c2ccccc2-c2ccccc2-c2ccc(N(c3cccc(-c4cccc5c4ccc4ccccc45)c3)c3cccc(-n4c5ccccc5c5ccccc54)c3)cc2)cc1]

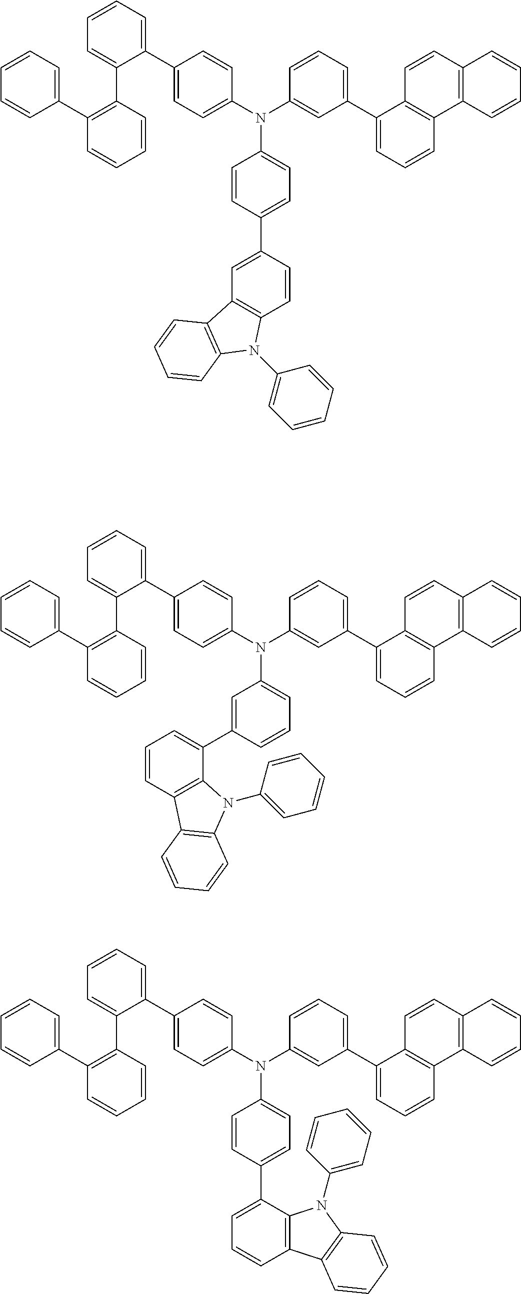 [SMILES: c1ccc(-c2ccccc2-c2ccccc2-c2ccc(N(c3ccc(-c4ccc5c(c4)c4ccccc4n5-c4ccccc4)cc3)c3cccc(-c4cccc5c4ccc4ccccc45)c3)cc2)cc1 ; c1ccc(-c2ccccc2-c2ccccc2-c2ccc(N(c3cccc(-c4cccc5c4ccc4ccccc45)c3)c3cccc(-c4cccc5c6ccccc6n(-c6ccccc6)c45)c3)cc2)cc1 ; c1ccc(-c2ccccc2-c2ccccc2-c2ccc(N(c3ccc(-c4cccc5c6ccccc6n(-c6ccccc6)c45)cc3)c3cccc(-c4cccc5c4ccc4ccccc45)c3)cc2)cc1]

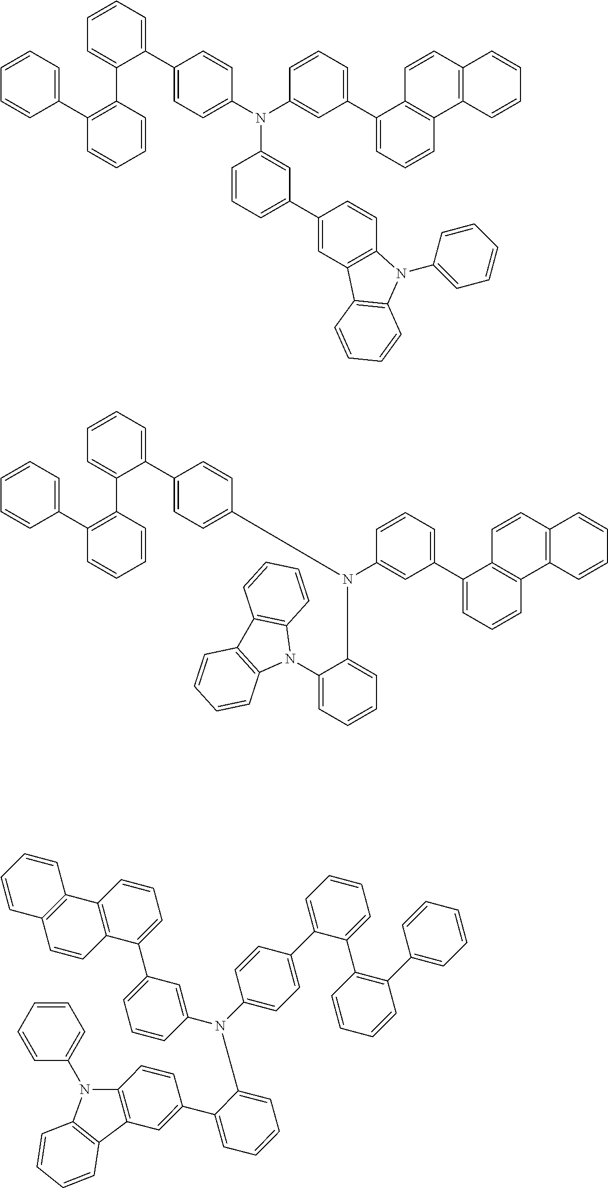 [SMILES: c1ccc(-c2ccccc2-c2ccccc2-c2ccc(N(c3cccc(-c4ccc5c(c4)c4ccccc4n5-c4ccccc4)c3)c3cccc(-c4cccc5c4ccc4ccccc45)c3)cc2)cc1 ; c1ccc(-c2ccccc2-c2ccccc2-c2ccc(N(c3cccc(-c4cccc5c4ccc4ccccc45)c3)c3ccccc3-n3c4ccccc4c4ccccc43)cc2)cc1 ; c1ccc(-c2ccccc2-c2ccccc2-c2ccc(N(c3cccc(-c4cccc5c4ccc4ccccc45)c3)c3ccccc3-c3ccc4c(c3)c3ccccc3n4-c3ccccc3)cc2)cc1]

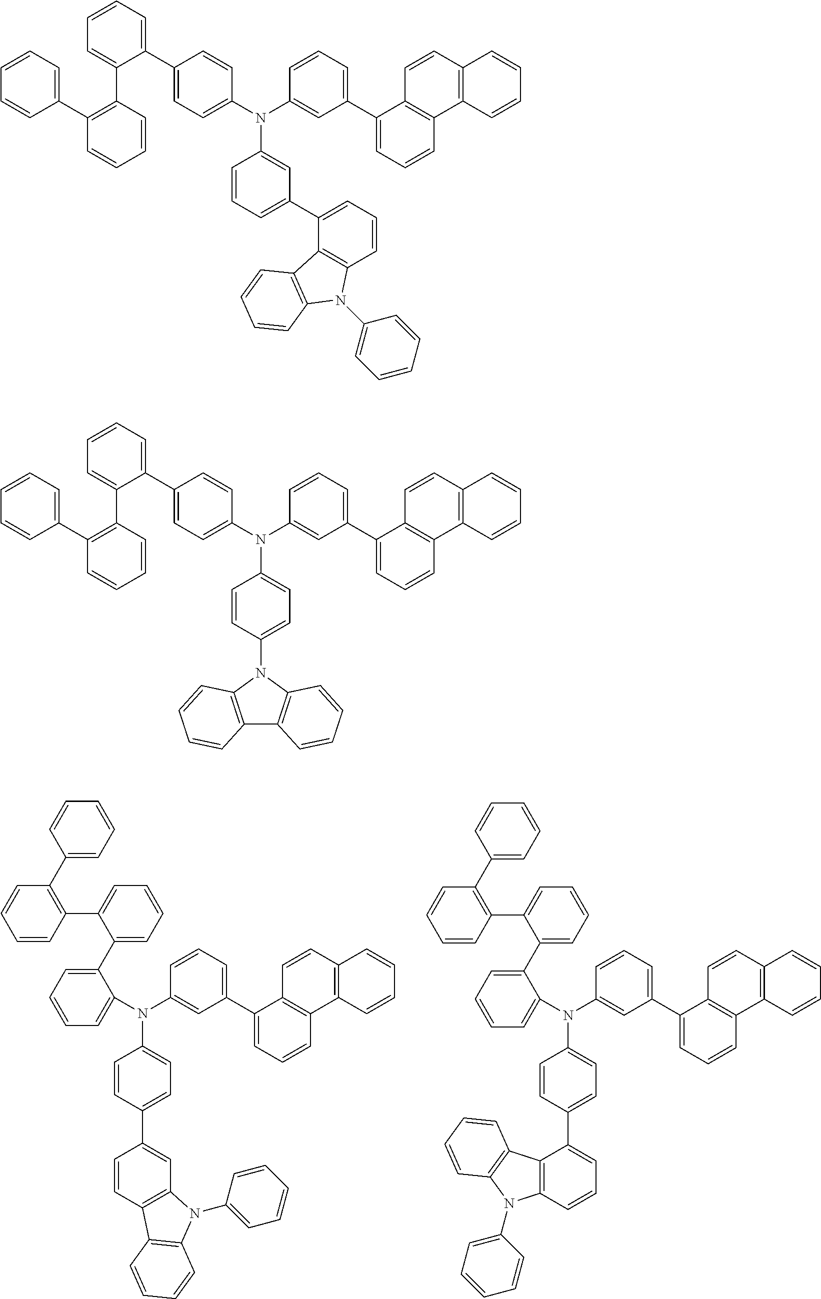 [SMILES: c1ccc(-c2ccccc2-c2ccccc2-c2ccc(N(c3cccc(-c4cccc5c4ccc4ccccc45)c3)c3cccc(-c4cccc5c4c4ccccc4n5-c4ccccc4)c3)cc2)cc1 ; c1ccc(-c2ccccc2-c2ccccc2-c2ccc(N(c3ccc(-n4c5ccccc5c5ccccc54)cc3)c3cccc(-c4cccc5c4ccc4ccccc45)c3)cc2)cc1 ; c1ccc(-c2ccccc2-c2ccccc2-c2ccccc2N(c2ccc(-c3ccc4c5ccccc5n(-c5ccccc5)c4c3)cc2)c2cccc(-c3cccc4c3ccc3ccccc34)c2)cc1 ; c1ccc(-c2ccccc2-c2ccccc2-c2ccccc2N(c2ccc(-c3cccc4c3c3ccccc3n4-c3ccccc3)cc2)c2cccc(-c3cccc4c3ccc3ccccc34)c2)cc1]

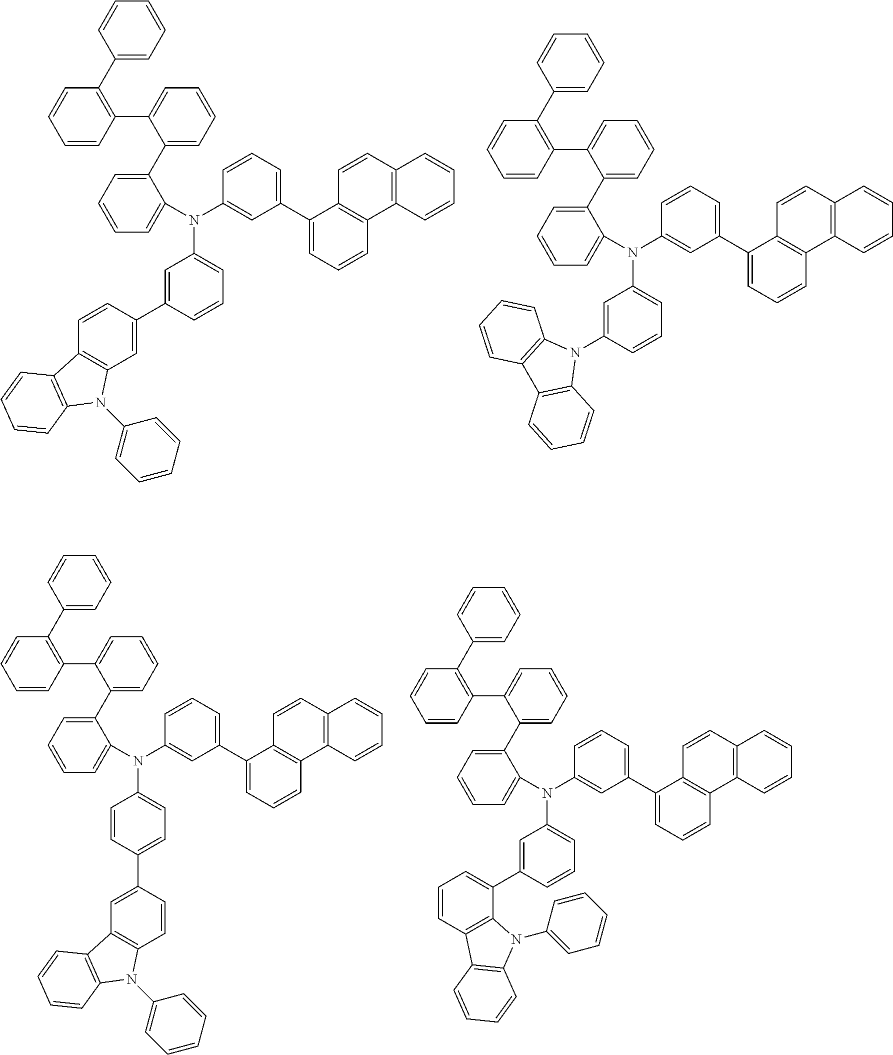

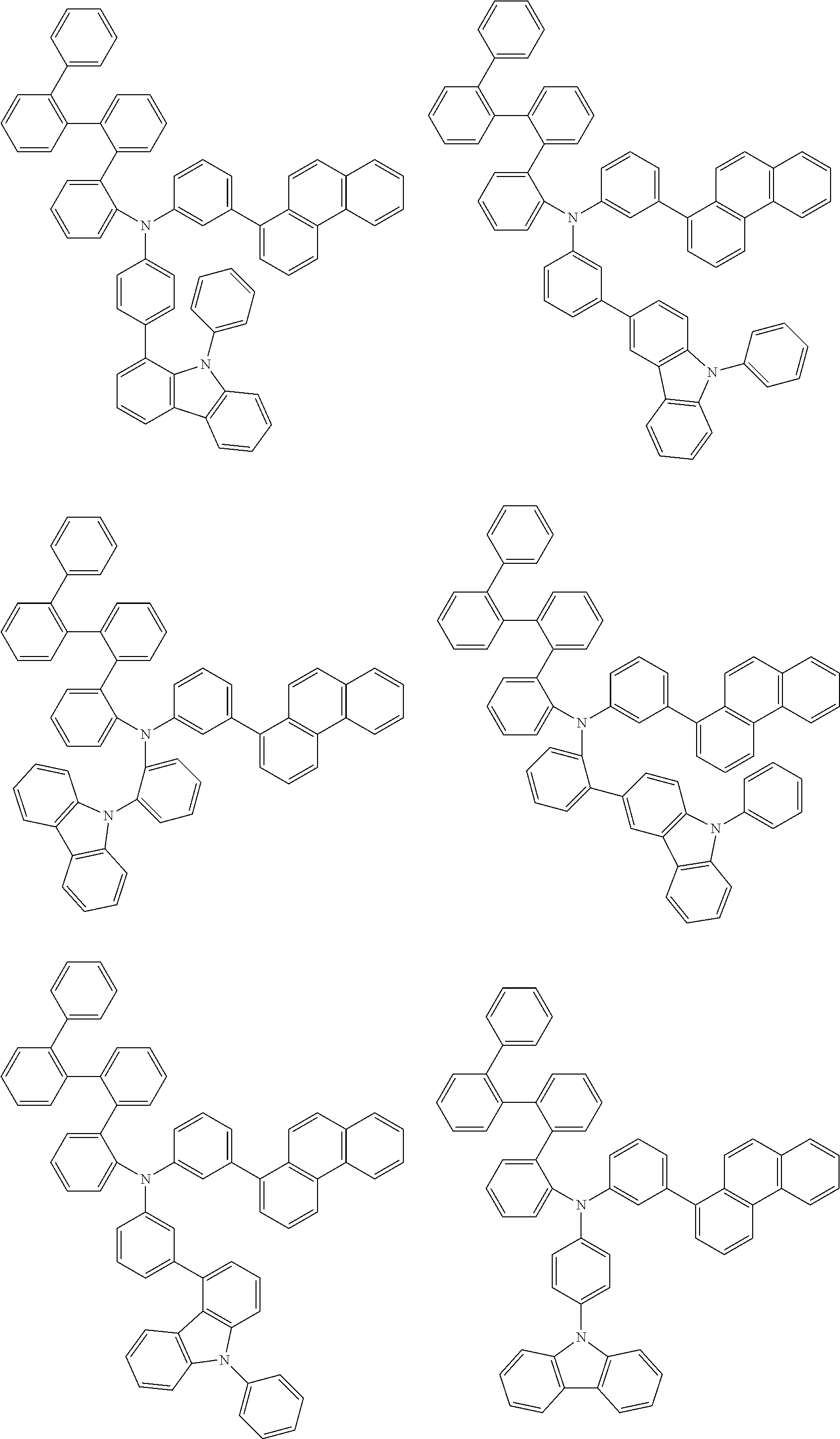

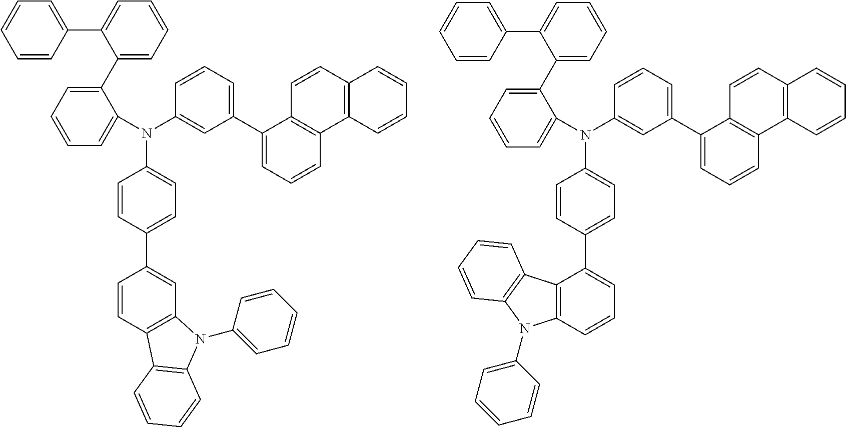 [SMILES: c1ccc(-c2ccccc2-c2ccccc2N(c2ccc(-c3ccc4c5ccccc5n(-c5ccccc5)c4c3)cc2)c2cccc(-c3cccc4c3ccc3ccccc34)c2)cc1 ; c1ccc(-c2ccccc2-c2ccccc2N(c2ccc(-c3cccc4c3c3ccccc3n4-c3ccccc3)cc2)c2cccc(-c3cccc4c3ccc3ccccc34)c2)cc1]

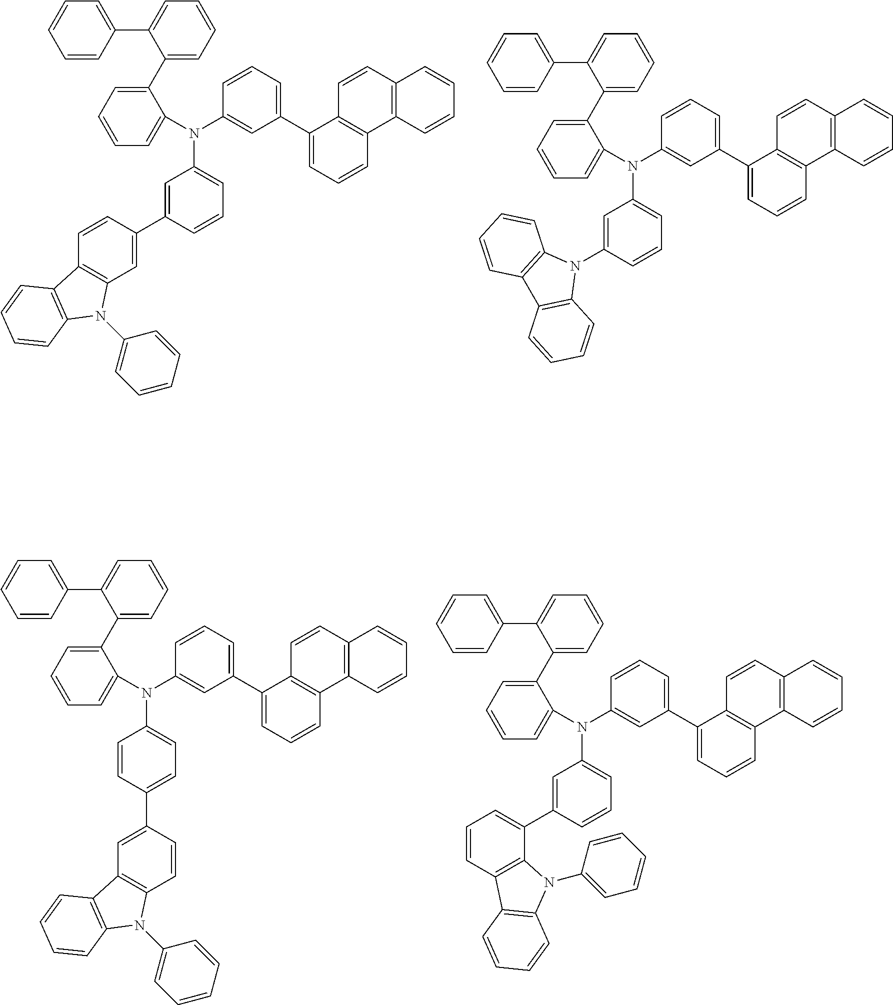

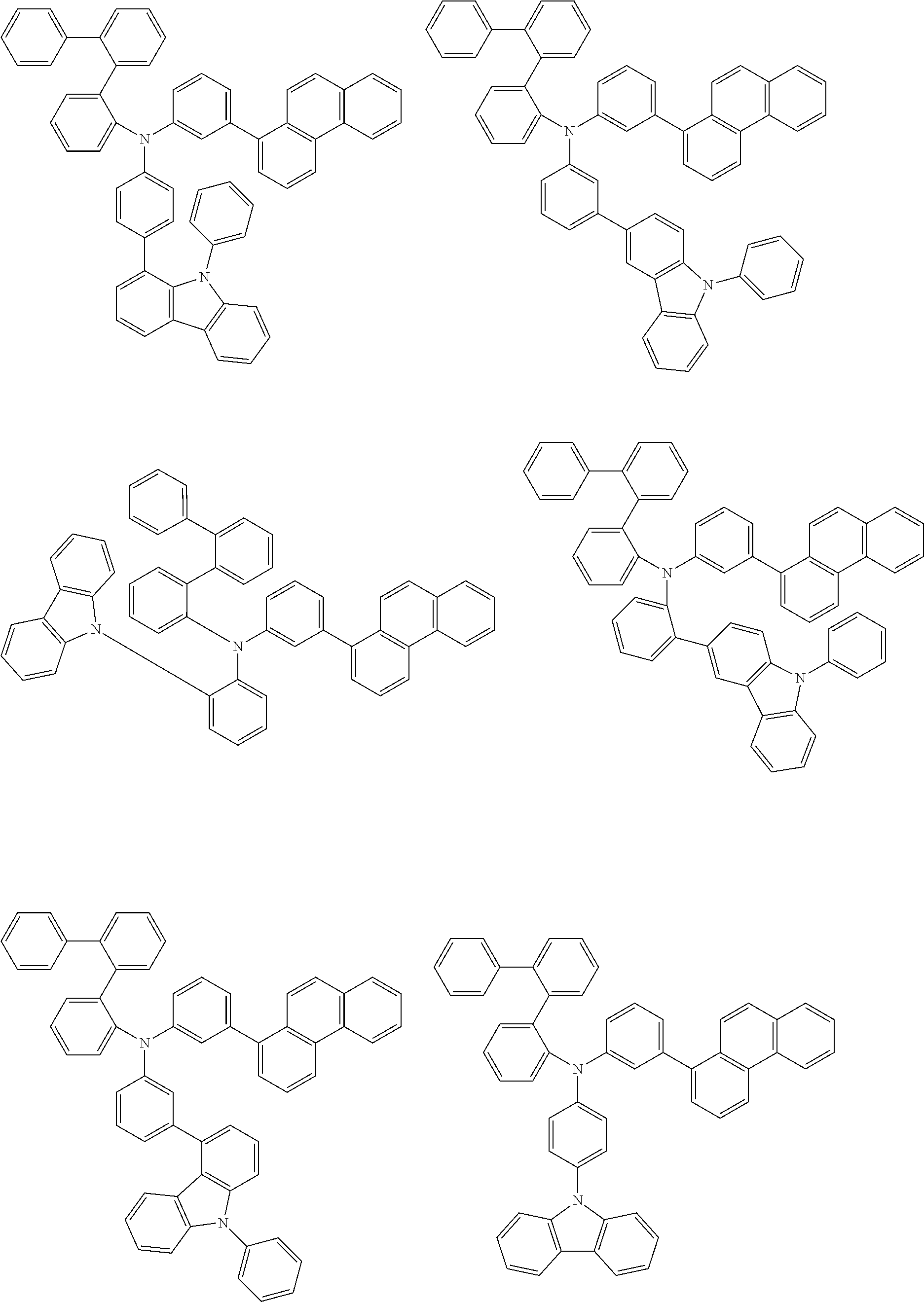

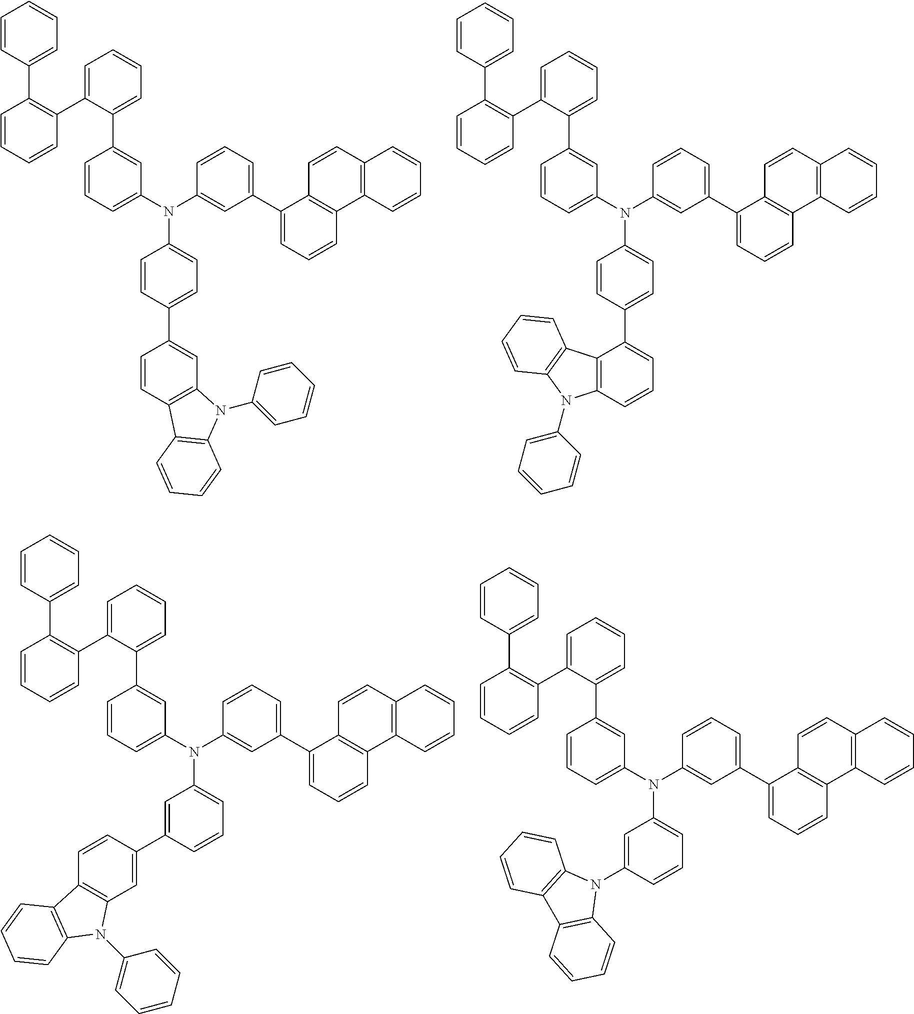

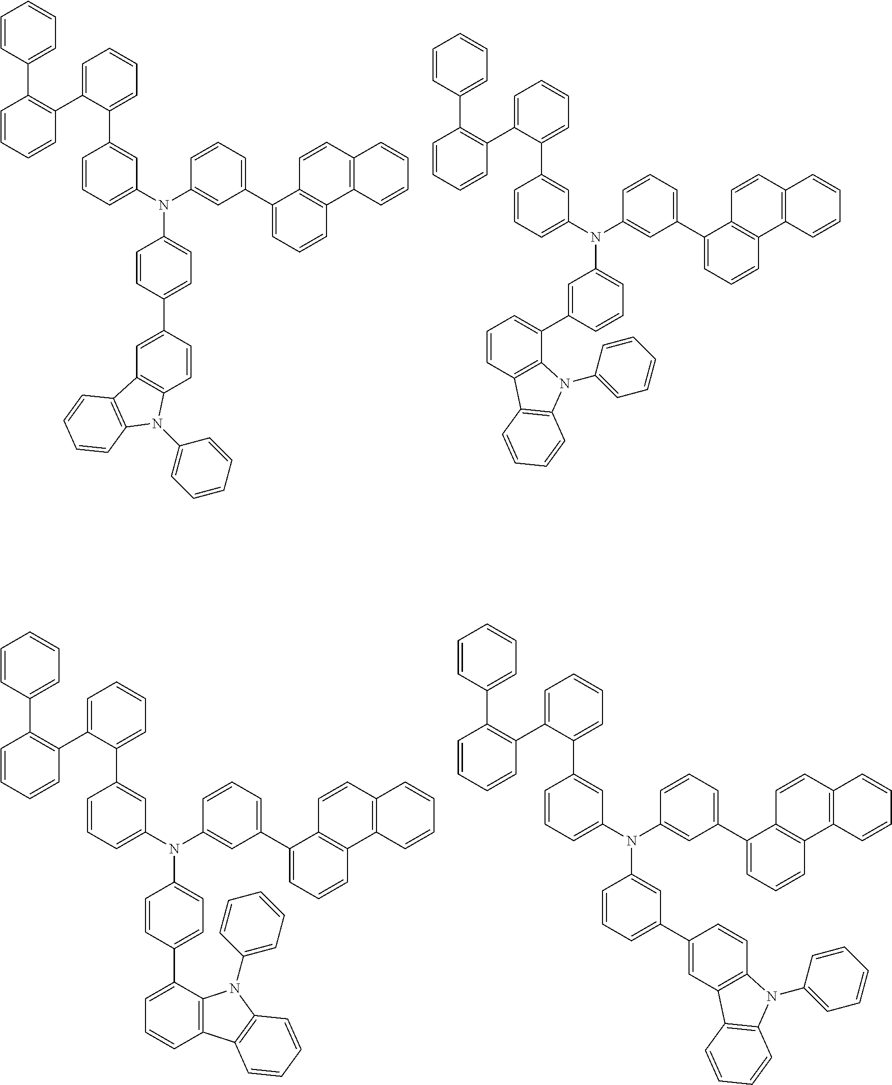

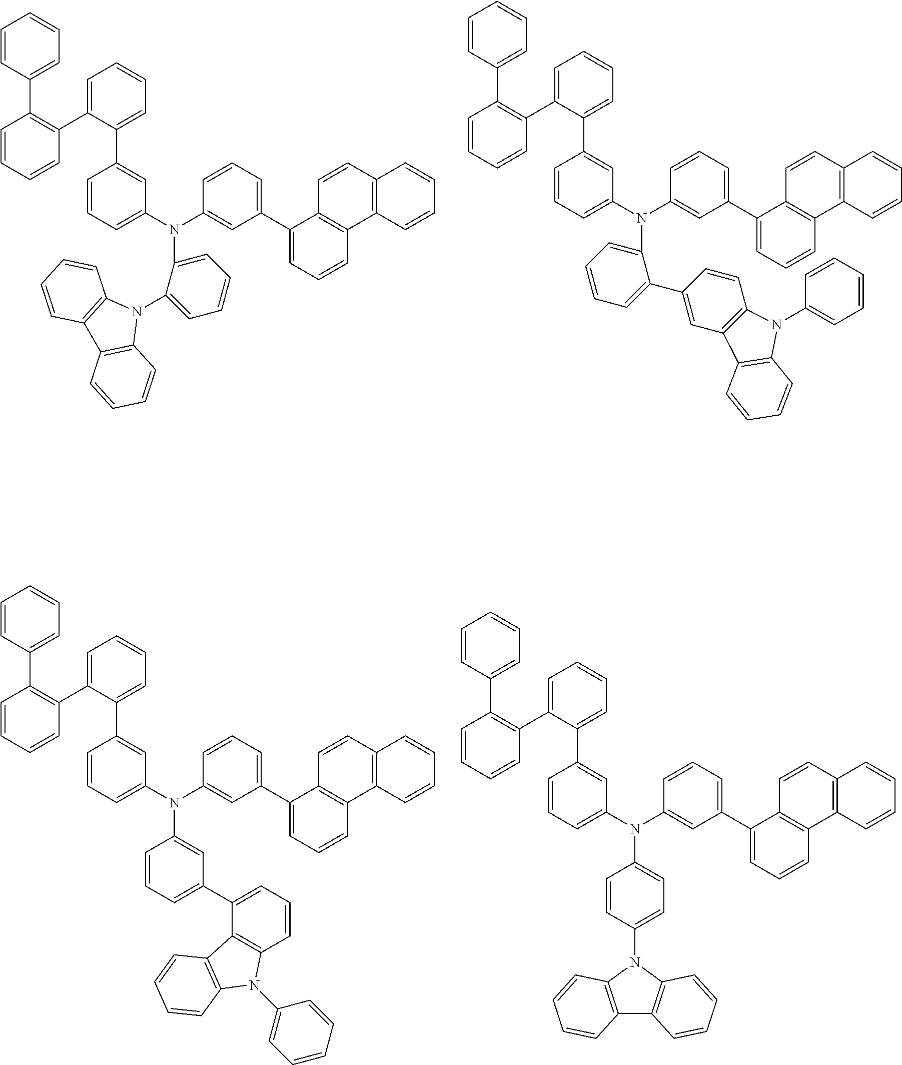

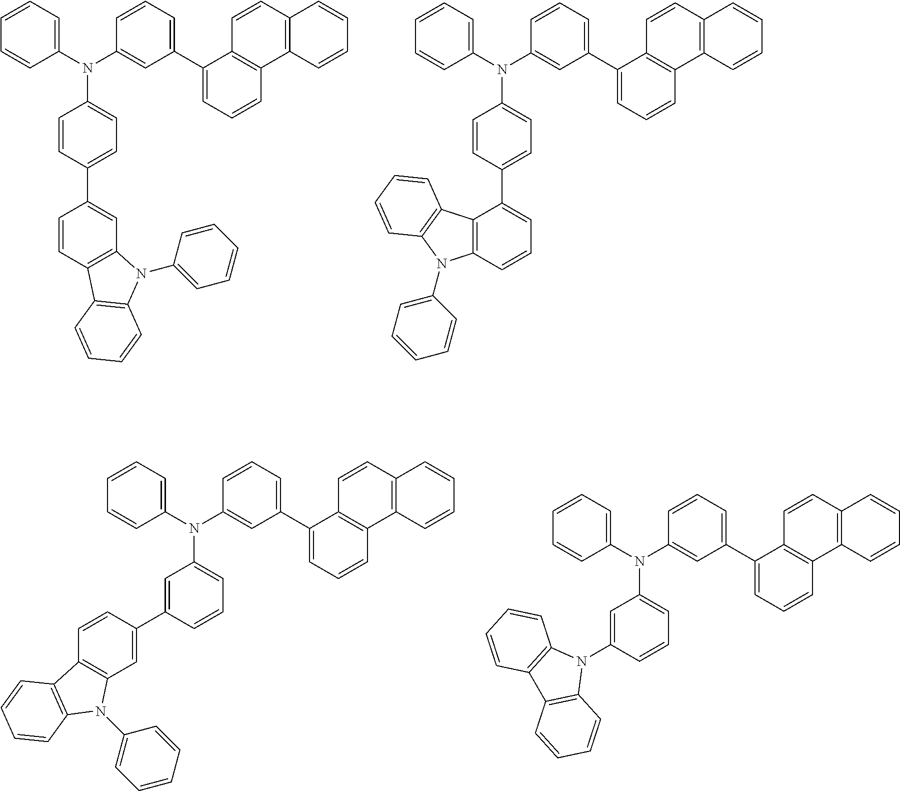 [SMILES: c1ccc(N(c2ccc(-c3ccc4c5ccccc5n(-c5ccccc5)c4c3)cc2)c2cccc(-c3cccc4c3ccc3ccccc34)c2)cc1 ; c1ccc(N(c2ccc(-c3cccc4c3c3ccccc3n4-c3ccccc3)cc2)c2cccc(-c3cccc4c3ccc3ccccc34)c2)cc1 ; c1ccc(N(c2cccc(-c3ccc4c5ccccc5n(-c5ccccc5)c4c3)c2)c2cccc(-c3cccc4c3ccc3ccccc34)c2)cc1 ; c1ccc(N(c2cccc(-c3cccc4c3ccc3ccccc34)c2)c2cccc(-n3c4ccccc4c4ccccc43)c2)cc1]

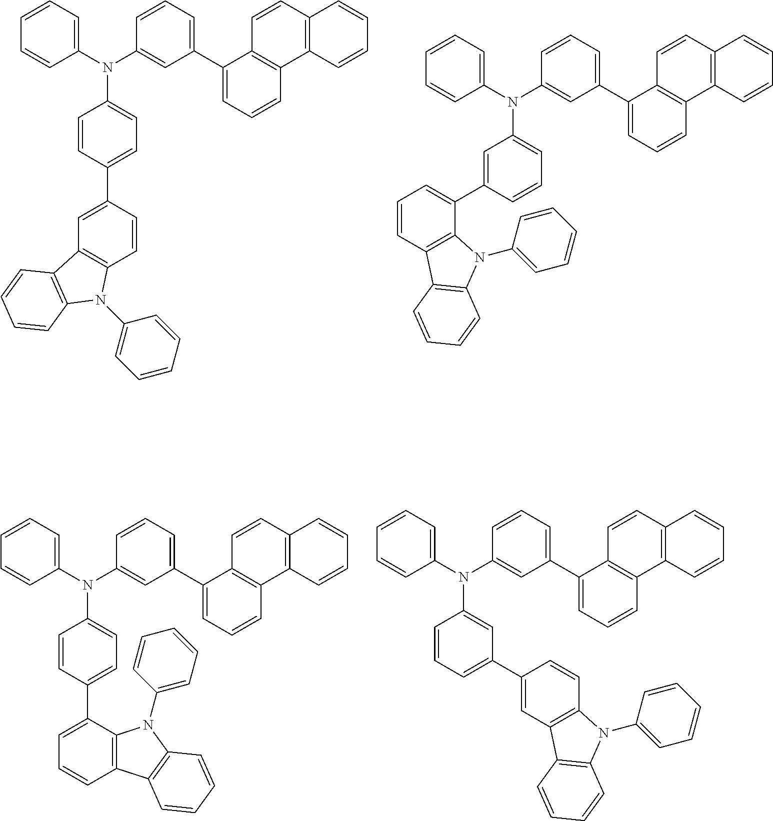

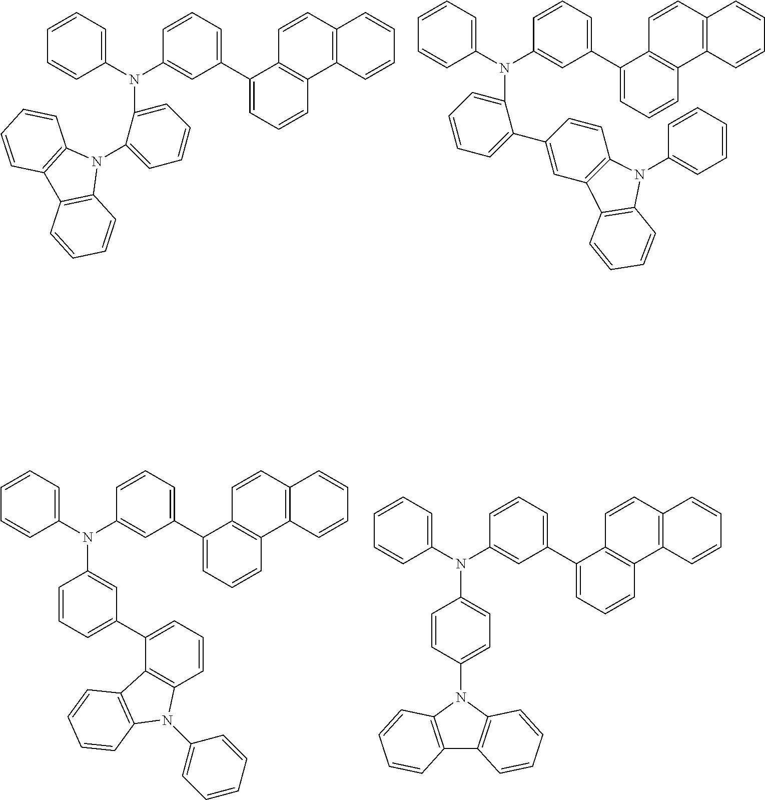

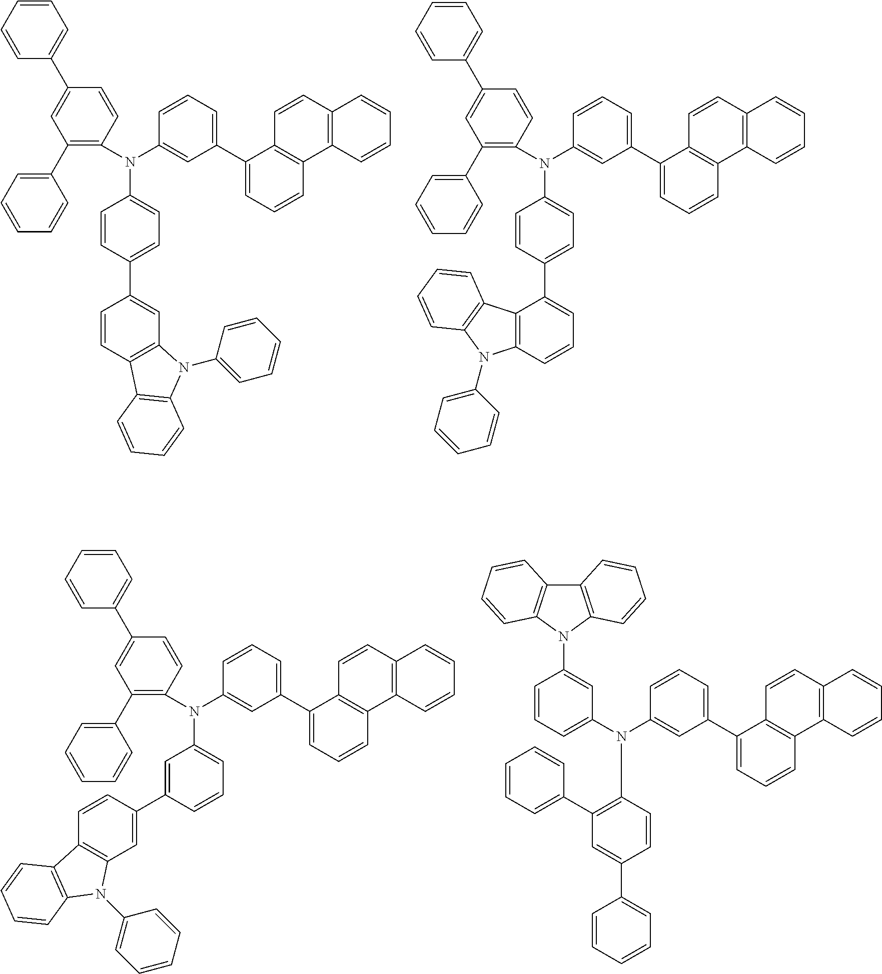 [SMILES: c1ccc(-c2ccc(N(c3ccc(-c4ccc5c6ccccc6n(-c6ccccc6)c5c4)cc3)c3cccc(-c4cccc5c4ccc4ccccc45)c3)c(-c3ccccc3)c2)cc1 ; c1ccc(-c2ccc(N(c3ccc(-c4cccc5c4c4ccccc4n5-c4ccccc4)cc3)c3cccc(-c4cccc5c4ccc4ccccc45)c3)c(-c3ccccc3)c2)cc1 ; c1ccc(-c2ccc(N(c3cccc(-c4ccc5c6ccccc6n(-c6ccccc6)c5c4)c3)c3cccc(-c4cccc5c4ccc4ccccc45)c3)c(-c3ccccc3)c2)cc1 ; c1ccc(-c2ccc(N(c3cccc(-c4cccc5c4ccc4ccccc45)c3)c3cccc(-n4c5ccccc5c5ccccc54)c3)c(-c3ccccc3)c2)cc1]

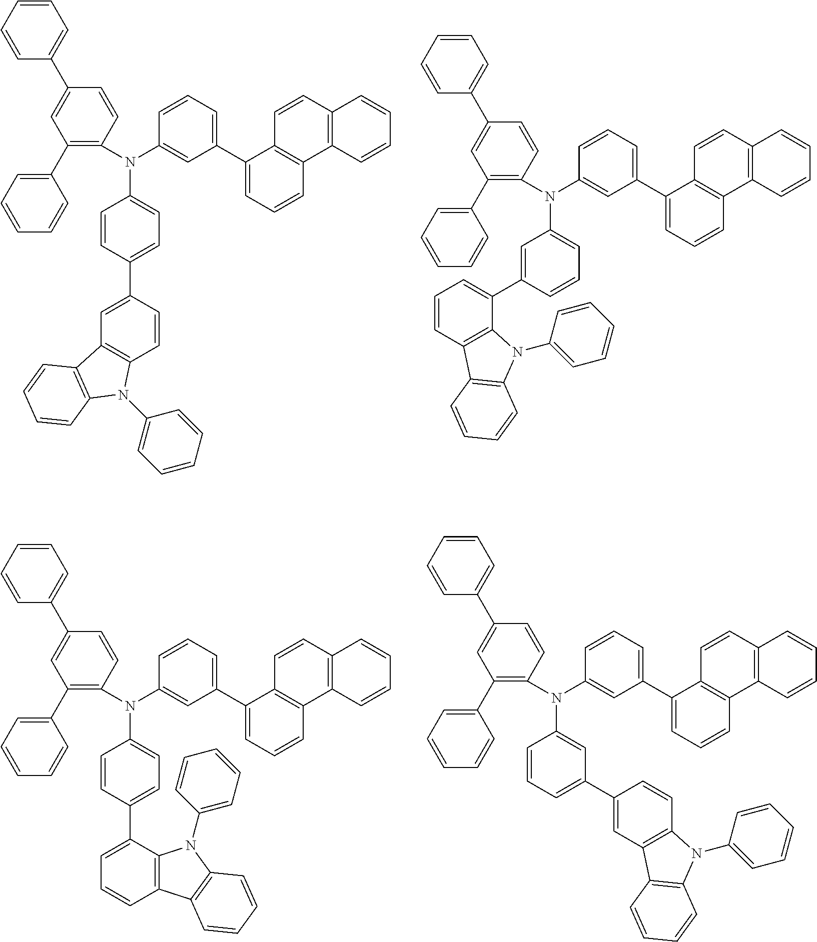 [SMILES: c1ccc(-c2ccc(N(c3ccc(-c4ccc5c(c4)c4ccccc4n5-c4ccccc4)cc3)c3cccc(-c4cccc5c4ccc4ccccc45)c3)c(-c3ccccc3)c2)cc1 ; c1ccc(-c2ccc(N(c3cccc(-c4cccc5c4ccc4ccccc45)c3)c3cccc(-c4cccc5c6ccccc6n(-c6ccccc6)c45)c3)c(-c3ccccc3)c2)cc1 ; c1ccc(-c2ccc(N(c3ccc(-c4cccc5c6ccccc6n(-c6ccccc6)c45)cc3)c3cccc(-c4cccc5c4ccc4ccccc45)c3)c(-c3ccccc3)c2)cc1 ; c1ccc(-c2ccc(N(c3cccc(-c4ccc5c(c4)c4ccccc4n5-c4ccccc4)c3)c3cccc(-c4cccc5c4ccc4ccccc45)c3)c(-c3ccccc3)c2)cc1]

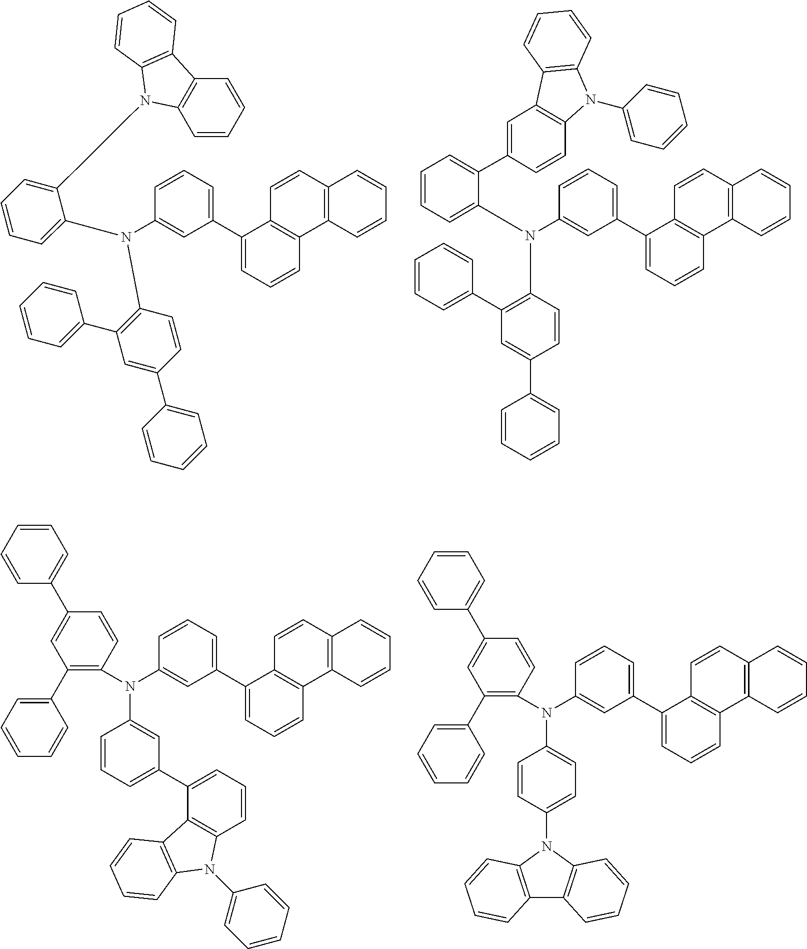

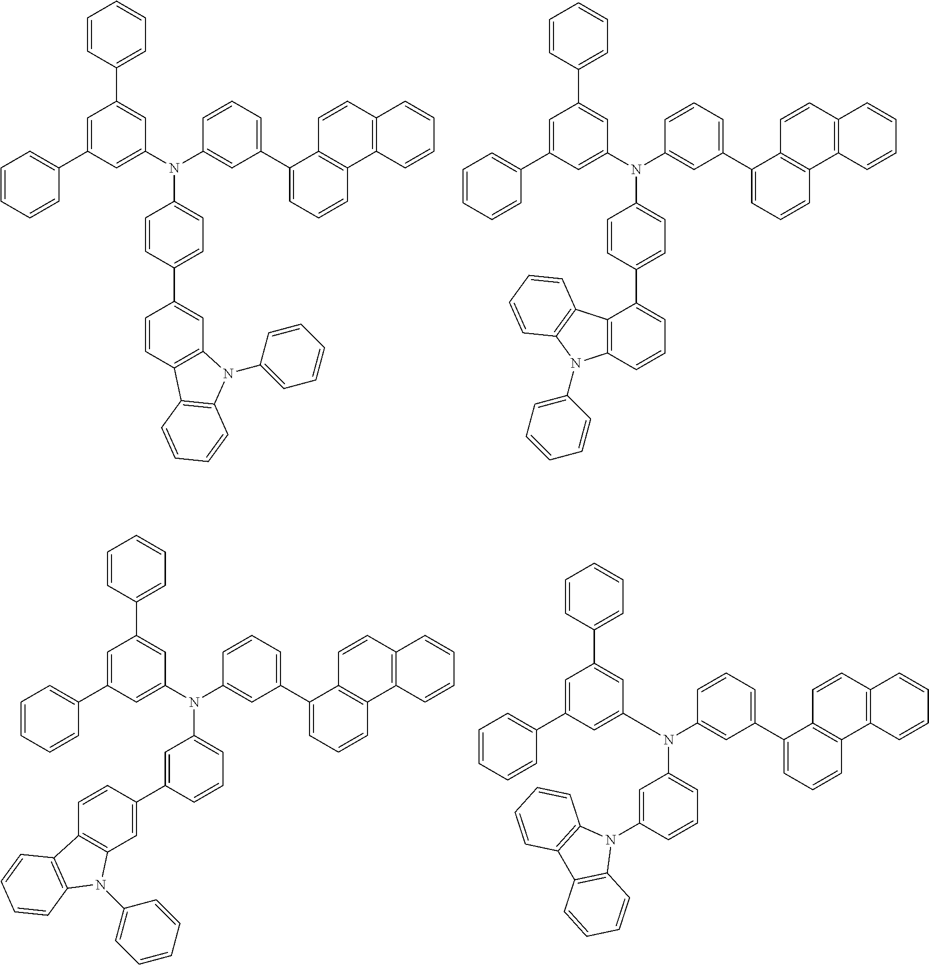

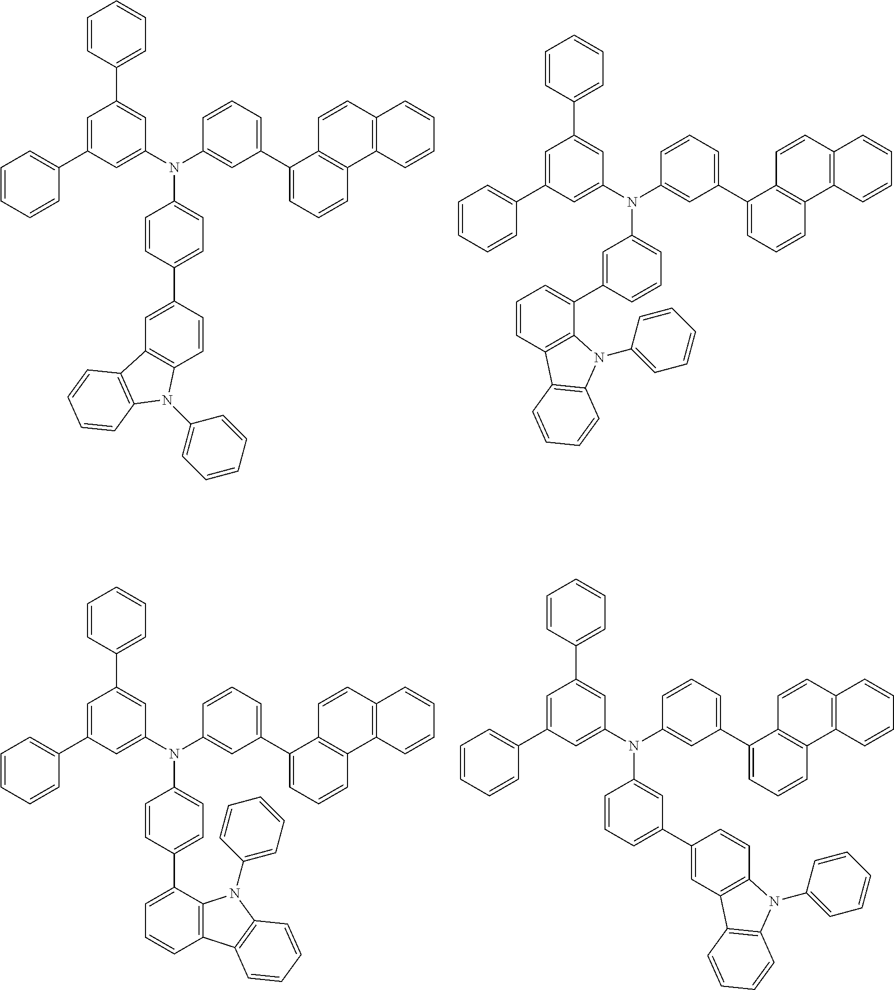

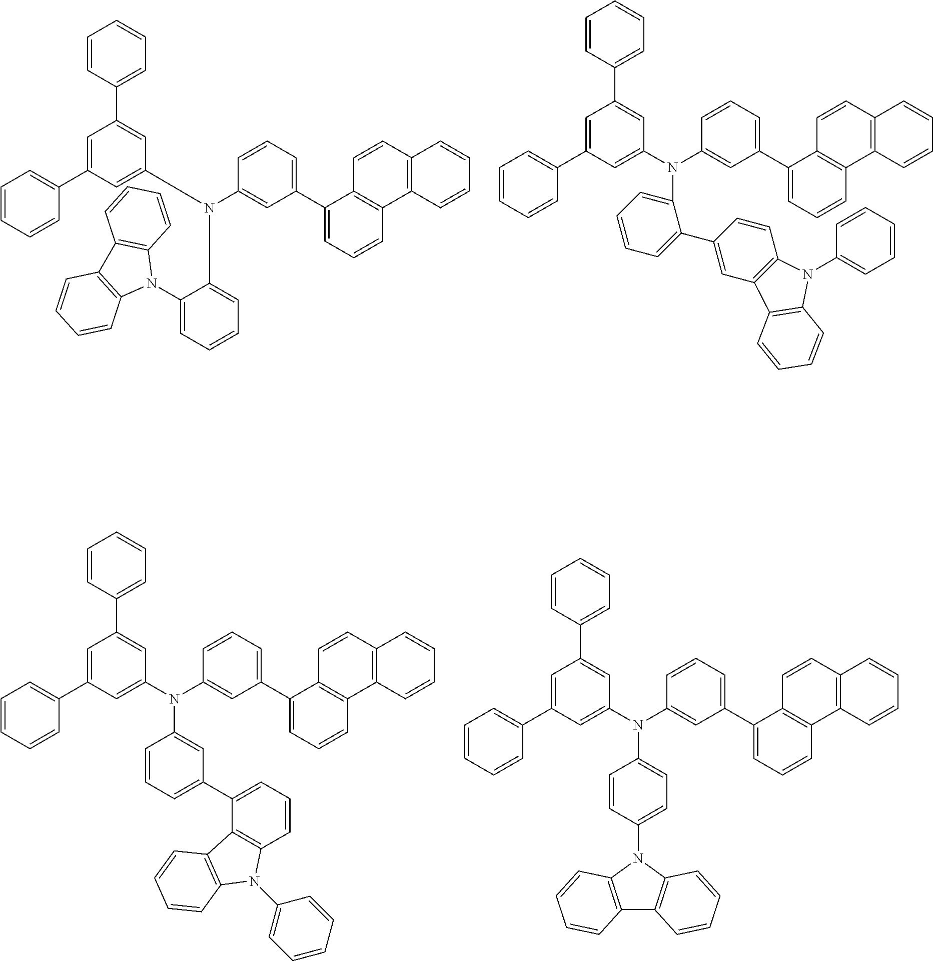

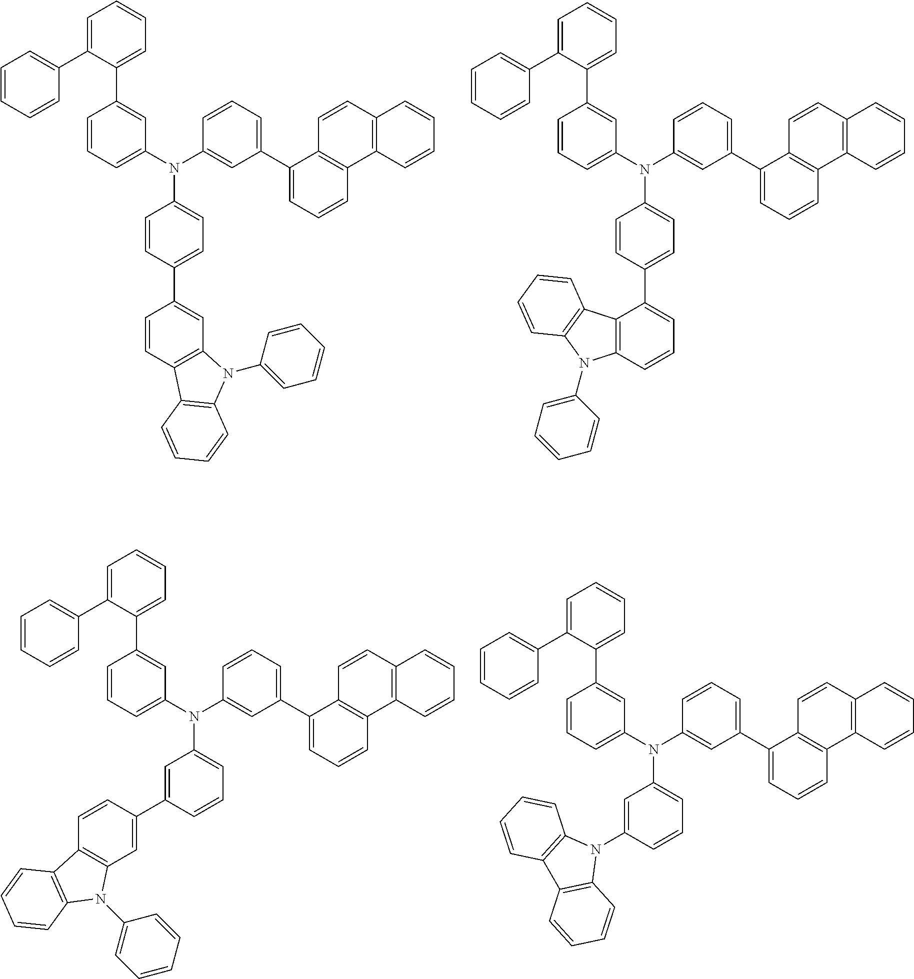

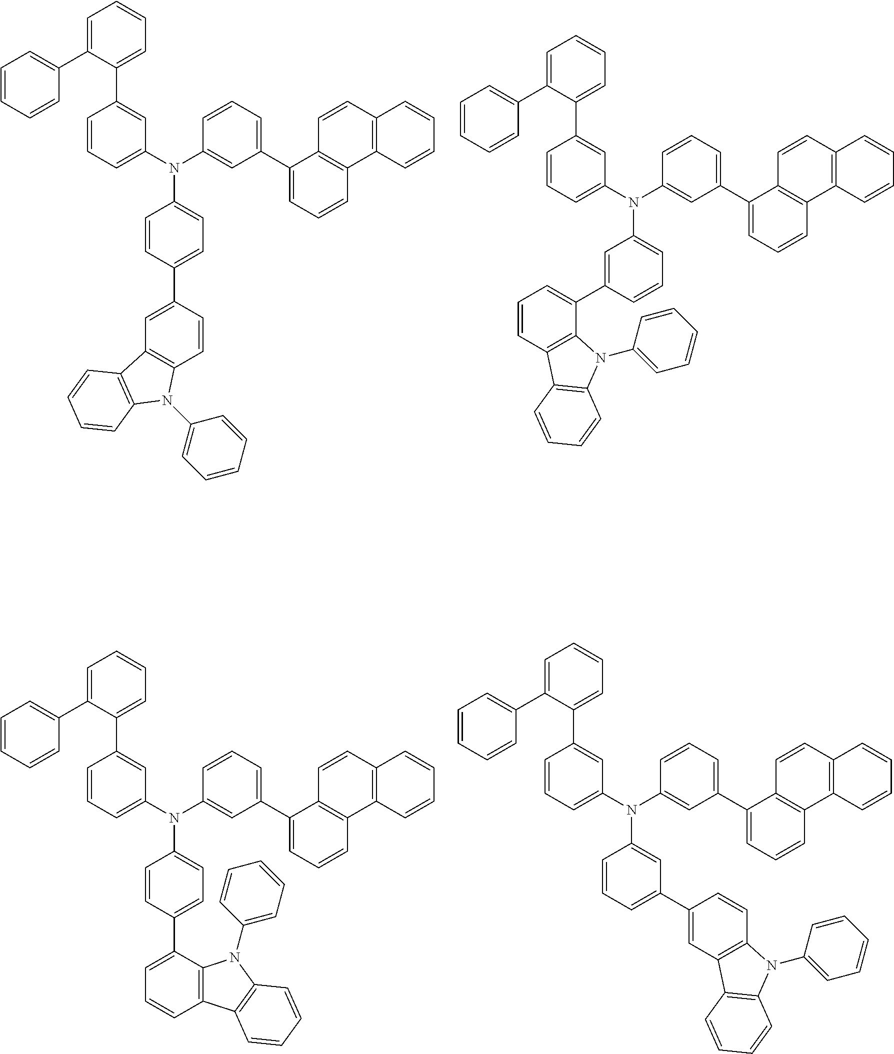

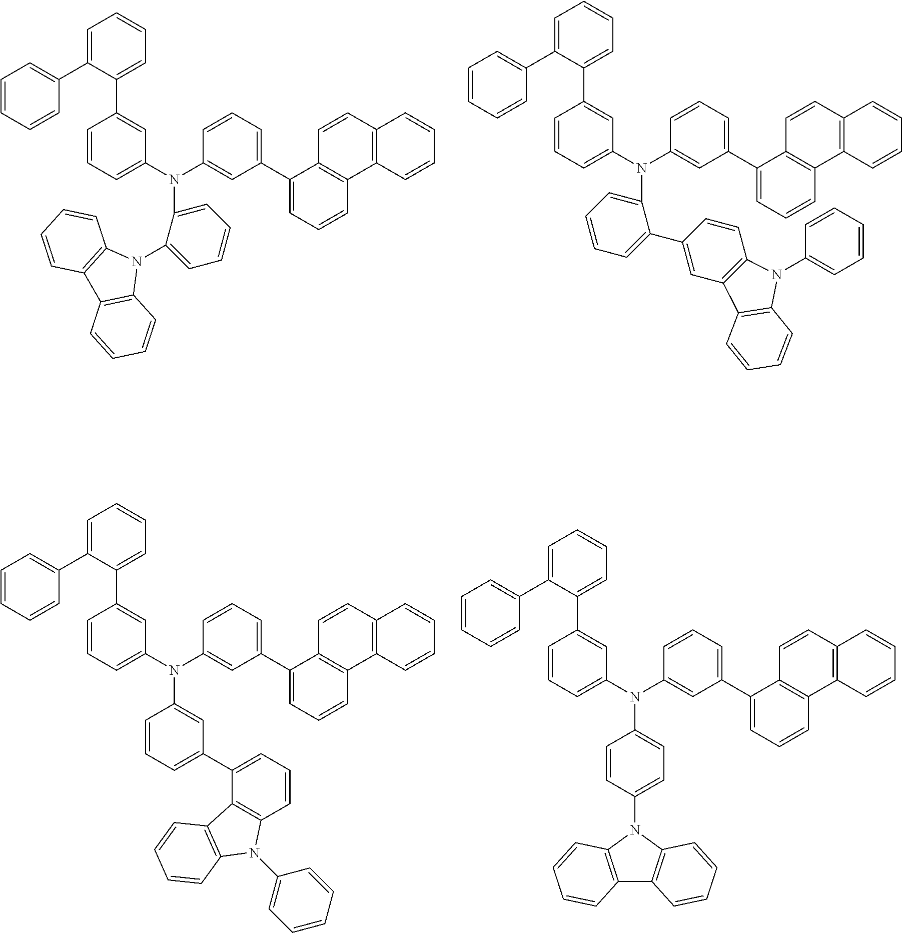

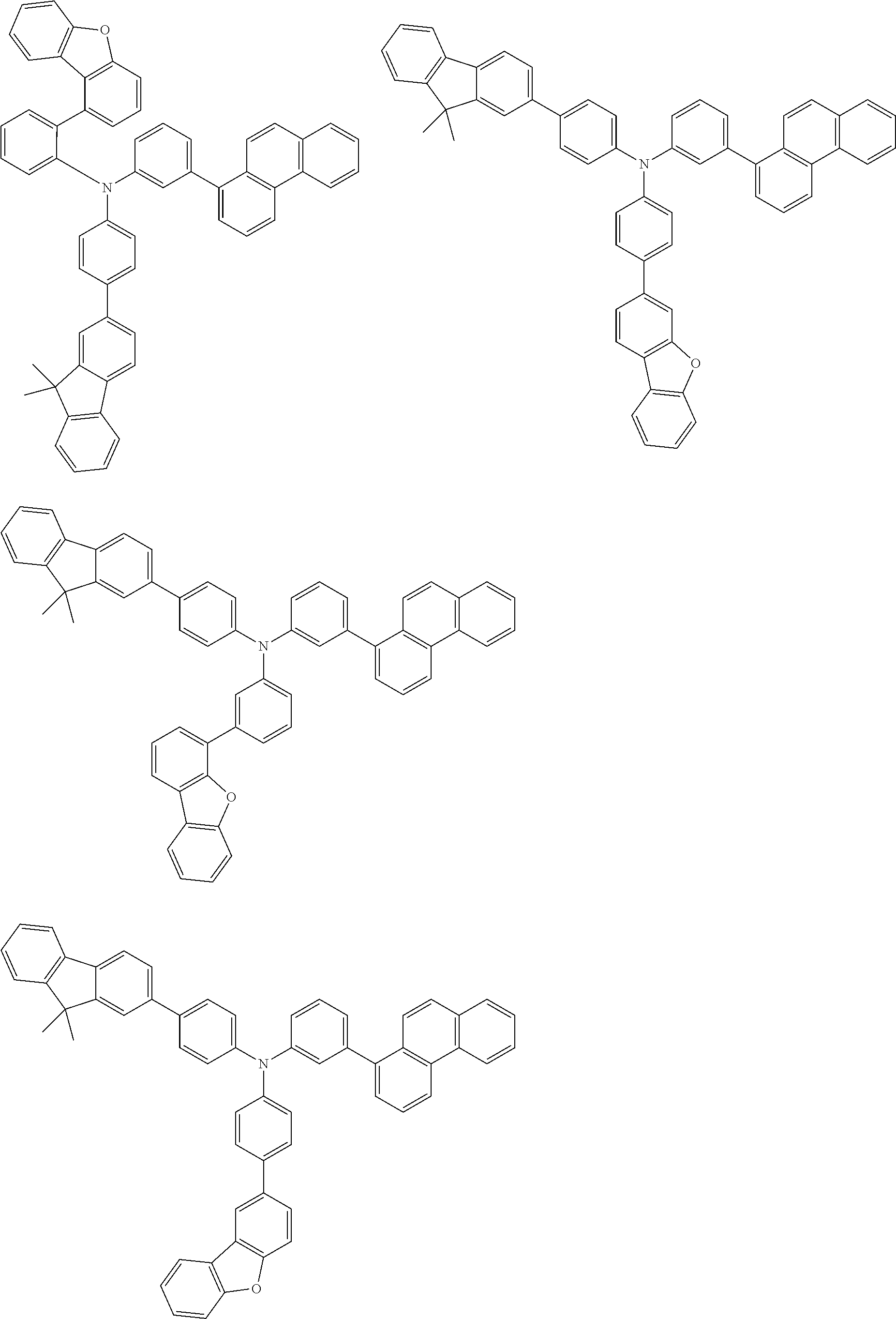

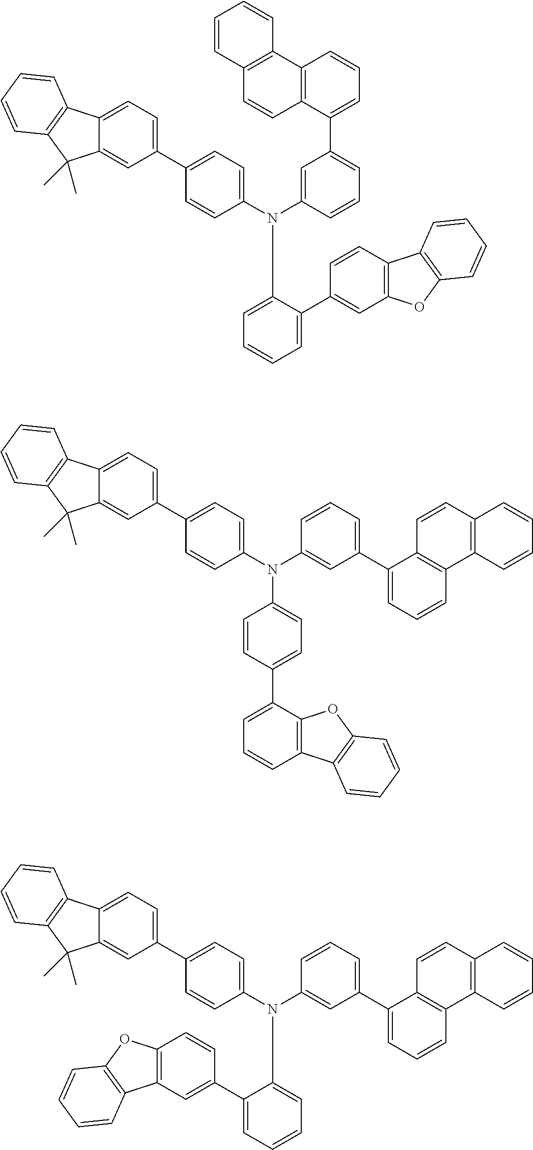 [SMILES: CC1(C)c2ccccc2-c2ccc(-c3ccc(N(c4cccc(-c5cccc6c5ccc5ccccc56)c4)c4ccccc4-c4ccc5c(c4)oc4ccccc45)cc3)cc21 ; CC1(C)c2ccccc2-c2ccc(-c3ccc(N(c4ccc(-c5cccc6c5oc5ccccc56)cc4)c4cccc(-c5cccc6c5ccc5ccccc56)c4)cc3)cc21 ; CC1(C)c2ccccc2-c2ccc(-c3ccc(N(c4cccc(-c5cccc6c5ccc5ccccc56)c4)c4ccccc4-c4ccc5oc6ccccc6c5c4)cc3)cc21]

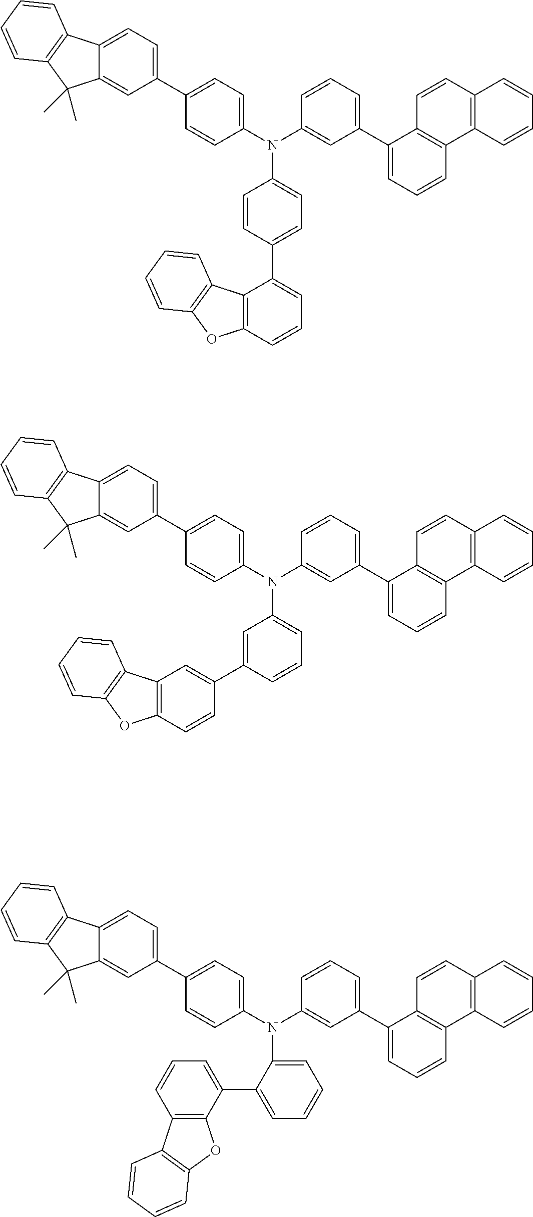 [SMILES: CC1(C)c2ccccc2-c2ccc(-c3ccc(N(c4ccc(-c5cccc6oc7ccccc7c56)cc4)c4cccc(-c5cccc6c5ccc5ccccc56)c4)cc3)cc21 ; CC1(C)c2ccccc2-c2ccc(-c3ccc(N(c4cccc(-c5ccc6oc7ccccc7c6c5)c4)c4cccc(-c5cccc6c5ccc5ccccc56)c4)cc3)cc21 ; CC1(C)c2ccccc2-c2ccc(-c3ccc(N(c4cccc(-c5cccc6c5ccc5ccccc56)c4)c4ccccc4-c4cccc5c4oc4ccccc45)cc3)cc21]

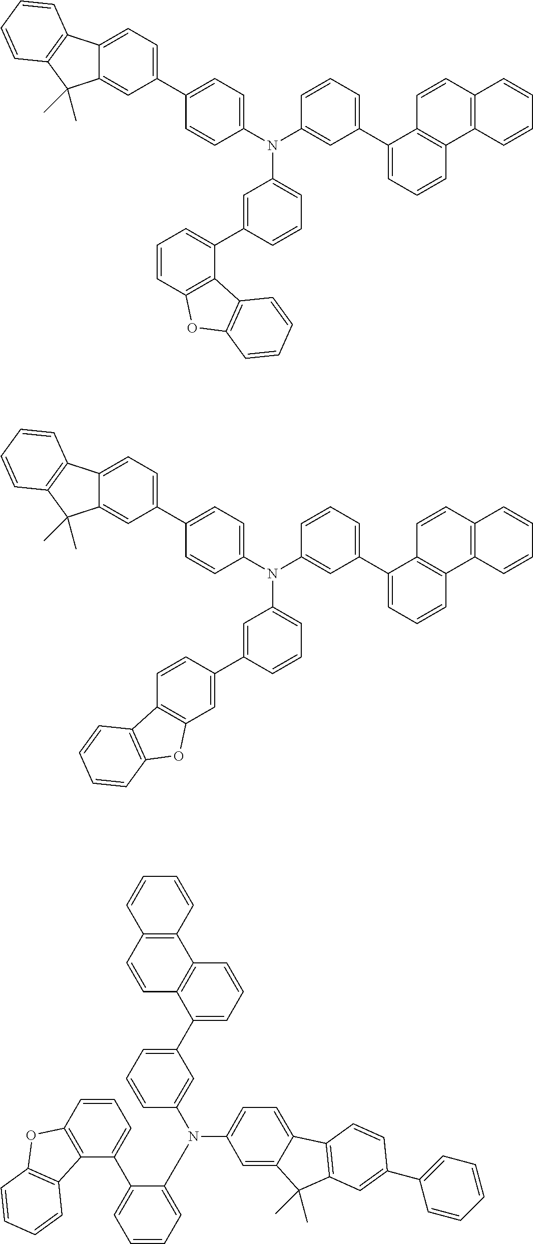 [SMILES: CC1(C)c2ccccc2-c2ccc(-c3ccc(N(c4cccc(-c5cccc6c5ccc5ccccc56)c4)c4cccc(-c5cccc6oc7ccccc7c56)c4)cc3)cc21 ; CC1(C)c2ccccc2-c2ccc(-c3ccc(N(c4cccc(-c5ccc6c(c5)oc5ccccc56)c4)c4cccc(-c5cccc6c5ccc5ccccc56)c4)cc3)cc21 ; CC1(C)c2cc(-c3ccccc3)ccc2-c2ccc(N(c3cccc(-c4cccc5c4ccc4ccccc45)c3)c3ccccc3-c3cccc4oc5ccccc5c34)cc21]

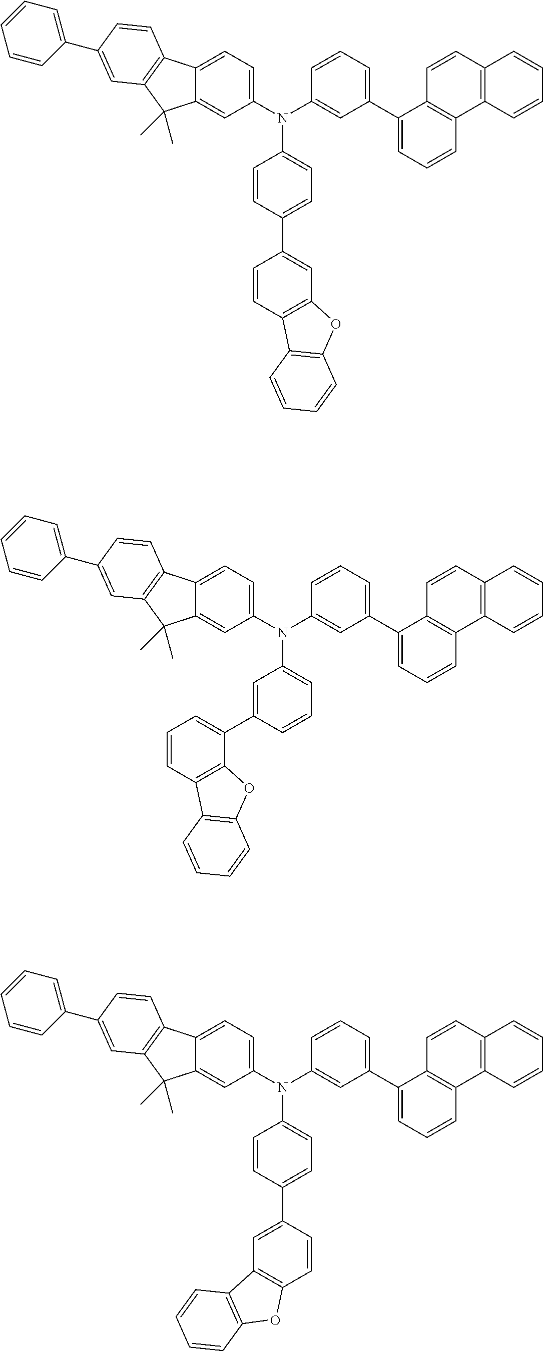 [SMILES: CC1(C)c2cc(-c3ccccc3)ccc2-c2ccc(N(c3ccc(-c4ccc5c(c4)oc4ccccc45)cc3)c3cccc(-c4cccc5c4ccc4ccccc45)c3)cc21 ; CC1(C)c2cc(-c3ccccc3)ccc2-c2ccc(N(c3cccc(-c4cccc5c4ccc4ccccc45)c3)c3cccc(-c4cccc5c4oc4ccccc45)c3)cc21 ; CC1(C)c2cc(-c3ccccc3)ccc2-c2ccc(N(c3ccc(-c4ccc5oc6ccccc6c5c4)cc3)c3cccc(-c4cccc5c4ccc4ccccc45)c3)cc21]

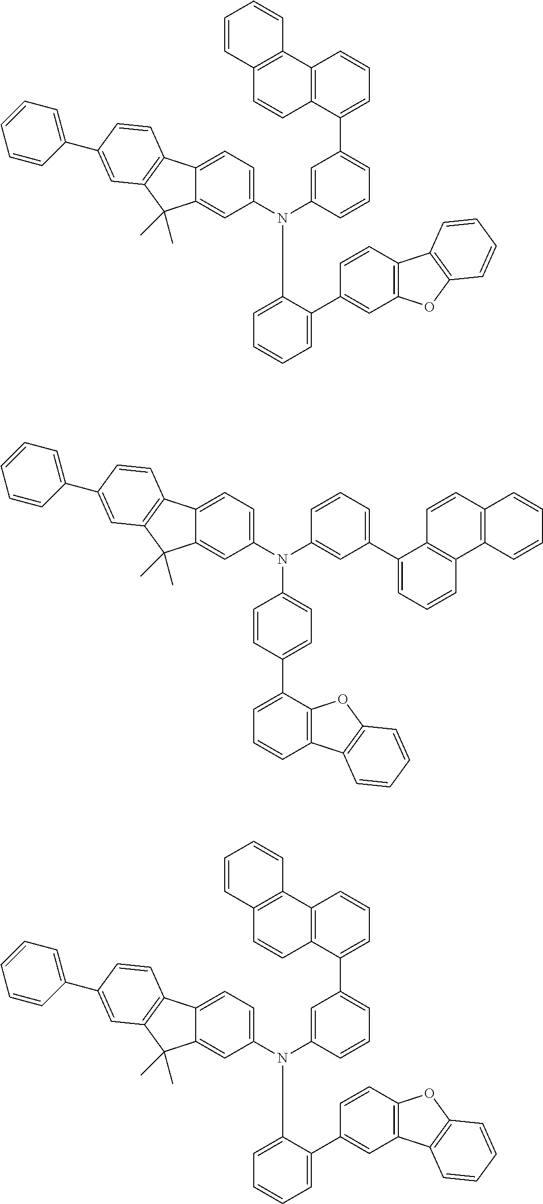 [SMILES: CC1(C)c2cc(-c3ccccc3)ccc2-c2ccc(N(c3cccc(-c4cccc5c4ccc4ccccc45)c3)c3ccccc3-c3ccc4c(c3)oc3ccccc34)cc21 ; CC1(C)c2cc(-c3ccccc3)ccc2-c2ccc(N(c3ccc(-c4cccc5c4oc4ccccc45)cc3)c3cccc(-c4cccc5c4ccc4ccccc45)c3)cc21 ; CC1(C)c2cc(-c3ccccc3)ccc2-c2ccc(N(c3cccc(-c4cccc5c4ccc4ccccc45)c3)c3ccccc3-c3ccc4oc5ccccc5c4c3)cc21]

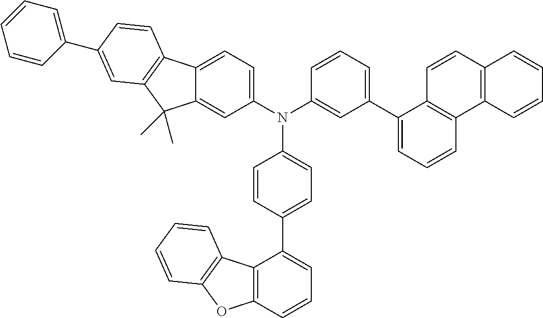 [SMILES: CC1(C)c2cc(-c3ccccc3)ccc2-c2ccc(N(c3ccc(-c4cccc5oc6ccccc6c45)cc3)c3cccc(-c4cccc5c4ccc4ccccc45)c3)cc21]

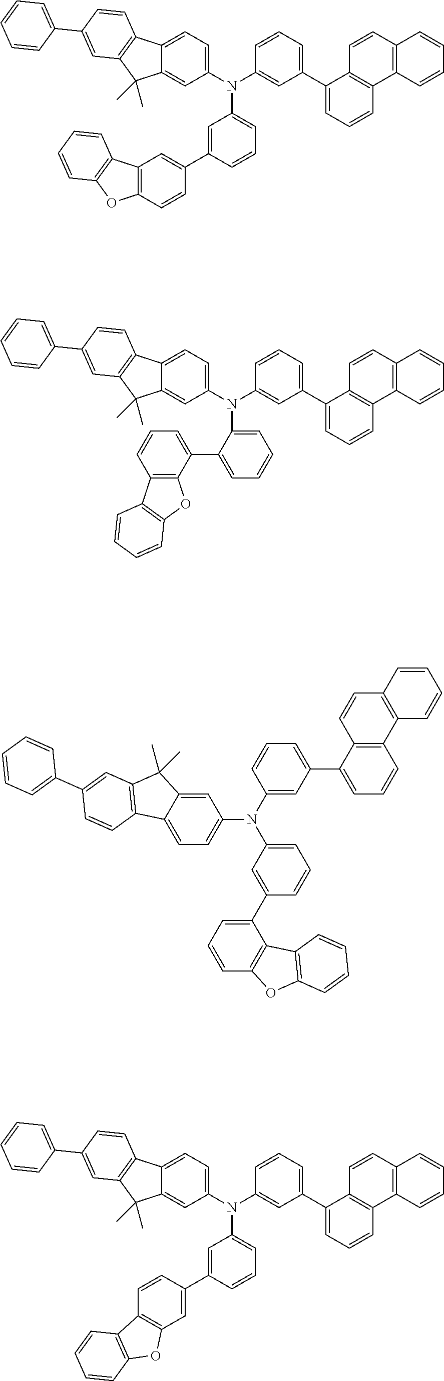 [SMILES: CC1(C)c2cc(-c3ccccc3)ccc2-c2ccc(N(c3cccc(-c4ccc5oc6ccccc6c5c4)c3)c3cccc(-c4cccc5c4ccc4ccccc45)c3)cc21 ; CC1(C)c2cc(-c3ccccc3)ccc2-c2ccc(N(c3cccc(-c4cccc5c4ccc4ccccc45)c3)c3ccccc3-c3cccc4c3oc3ccccc34)cc21 ; CC1(C)c2cc(-c3ccccc3)ccc2-c2ccc(N(c3cccc(-c4cccc5c4ccc4ccccc45)c3)c3cccc(-c4cccc5oc6ccccc6c45)c3)cc21 ; CC1(C)c2cc(-c3ccccc3)ccc2-c2ccc(N(c3cccc(-c4ccc5c(c4)oc4ccccc45)c3)c3cccc(-c4cccc5c4ccc4ccccc45)c3)cc21]

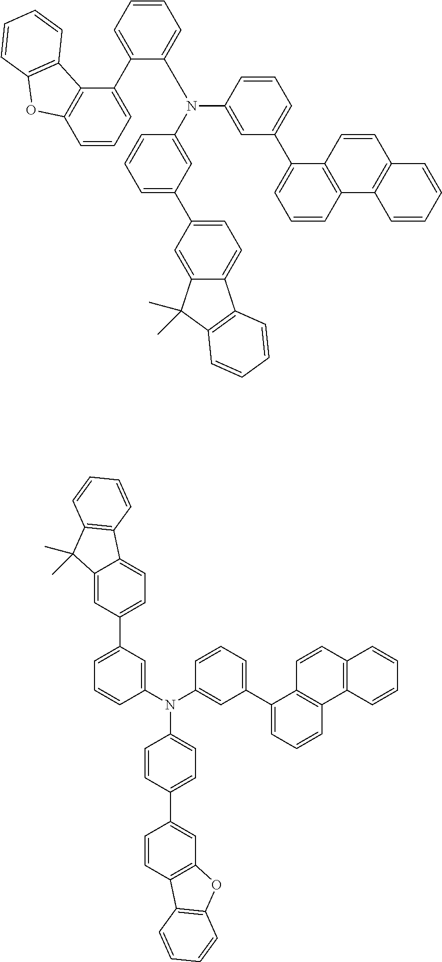 [SMILES: CC1(C)c2ccccc2-c2ccc(-c3cccc(N(c4cccc(-c5cccc6c5ccc5ccccc56)c4)c4ccccc4-c4cccc5oc6ccccc6c45)c3)cc21 ; CC1(C)c2ccccc2-c2ccc(-c3cccc(N(c4ccc(-c5ccc6c(c5)oc5ccccc56)cc4)c4cccc(-c5cccc6c5ccc5ccccc56)c4)c3)cc21]

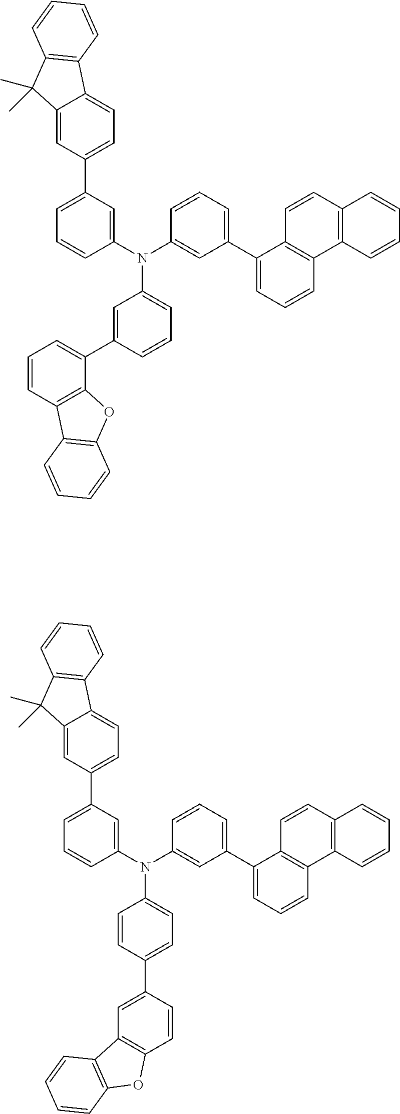 [SMILES: CC1(C)c2ccccc2-c2ccc(-c3cccc(N(c4cccc(-c5cccc6c5ccc5ccccc56)c4)c4cccc(-c5cccc6c5oc5ccccc56)c4)c3)cc21 ; CC1(C)c2ccccc2-c2ccc(-c3cccc(N(c4ccc(-c5ccc6oc7ccccc7c6c5)cc4)c4cccc(-c5cccc6c5ccc5ccccc56)c4)c3)cc21]

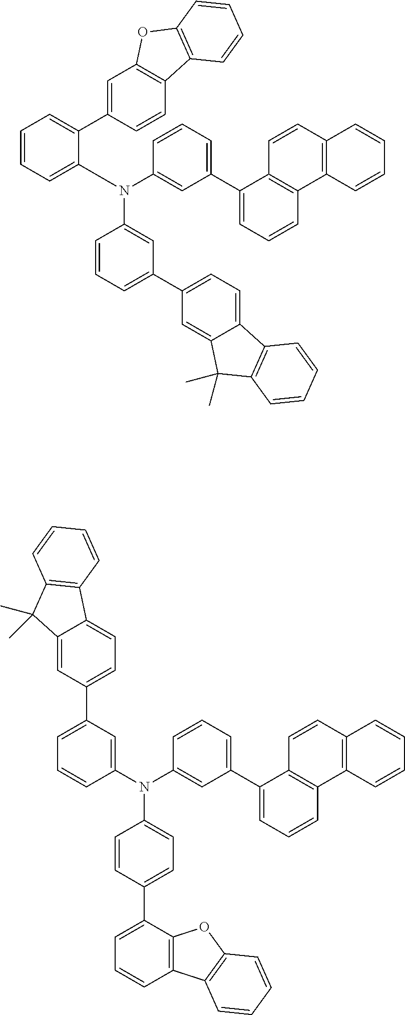 [SMILES: CC1(C)c2ccccc2-c2ccc(-c3cccc(N(c4cccc(-c5cccc6c5ccc5ccccc56)c4)c4ccccc4-c4ccc5c(c4)oc4ccccc45)c3)cc21 ; CC1(C)c2ccccc2-c2ccc(-c3cccc(N(c4ccc(-c5cccc6c5oc5ccccc56)cc4)c4cccc(-c5cccc6c5ccc5ccccc56)c4)c3)cc21]

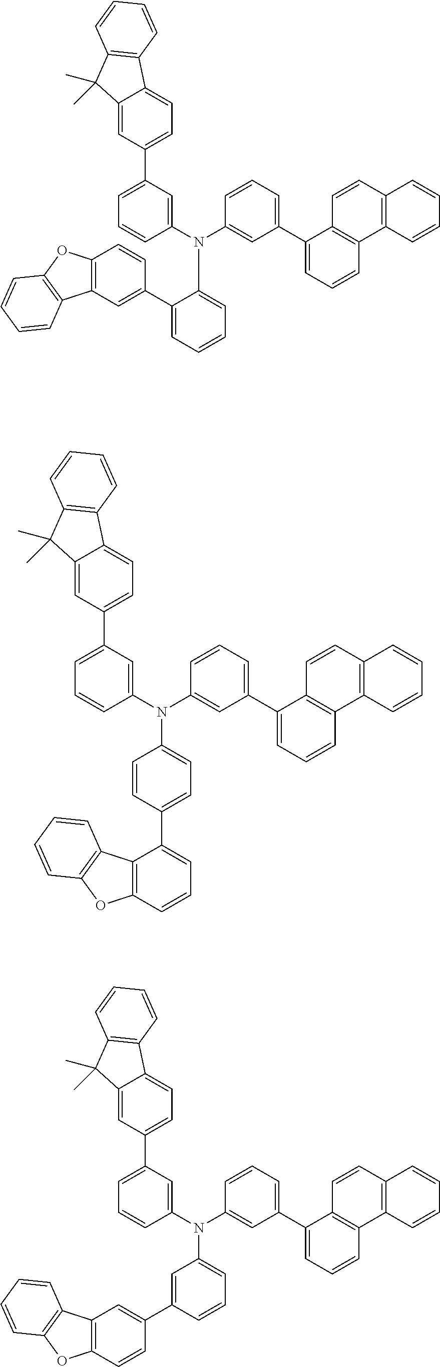 [SMILES: CC1(C)c2ccccc2-c2ccc(-c3cccc(N(c4cccc(-c5cccc6c5ccc5ccccc56)c4)c4ccccc4-c4ccc5oc6ccccc6c5c4)c3)cc21 ; CC1(C)c2ccccc2-c2ccc(-c3cccc(N(c4ccc(-c5cccc6oc7ccccc7c56)cc4)c4cccc(-c5cccc6c5ccc5ccccc56)c4)c3)cc21 ; CC1(C)c2ccccc2-c2ccc(-c3cccc(N(c4cccc(-c5ccc6oc7ccccc7c6c5)c4)c4cccc(-c5cccc6c5ccc5ccccc56)c4)c3)cc21]

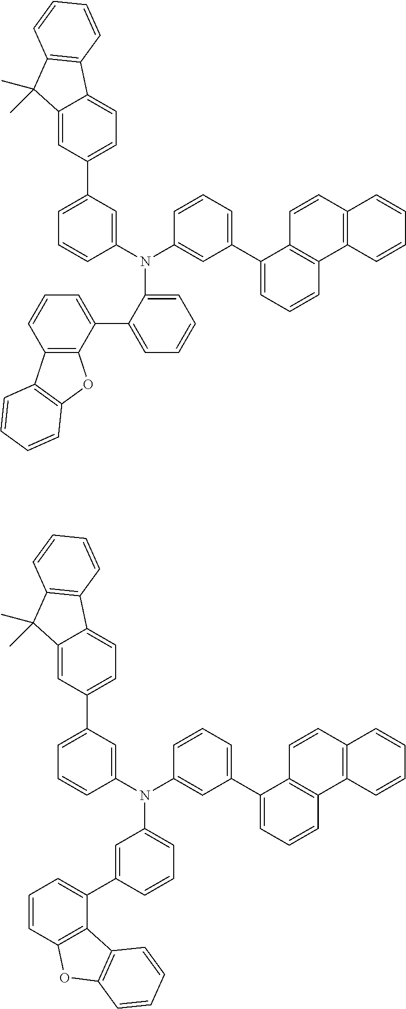 [SMILES: CC1(C)c2ccccc2-c2ccc(-c3cccc(N(c4cccc(-c5cccc6c5ccc5ccccc56)c4)c4ccccc4-c4cccc5c4oc4ccccc45)c3)cc21 ; CC1(C)c2ccccc2-c2ccc(-c3cccc(N(c4cccc(-c5cccc6c5ccc5ccccc56)c4)c4cccc(-c5cccc6oc7ccccc7c56)c4)c3)cc21]

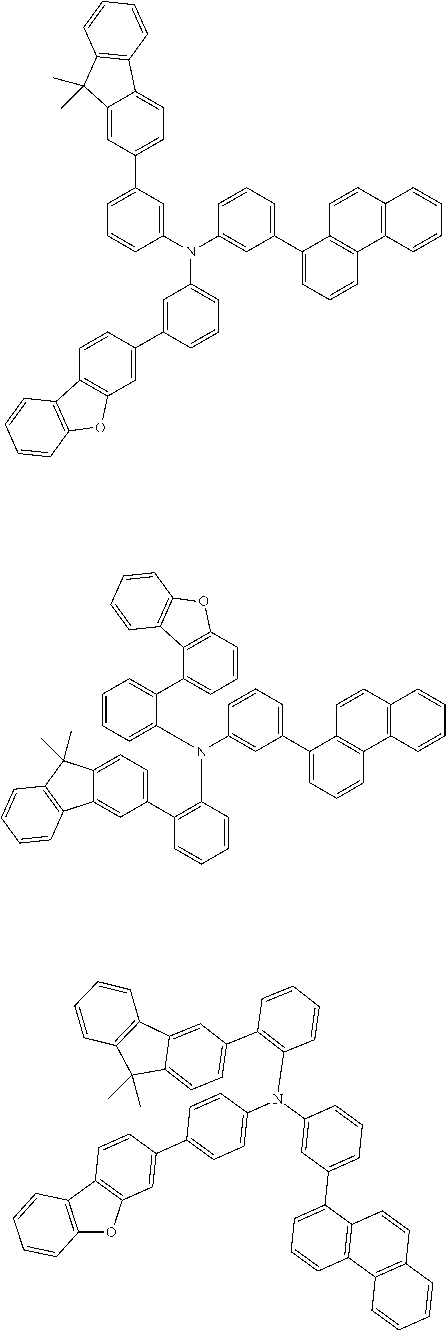 [SMILES: CC1(C)c2ccccc2-c2ccc(-c3cccc(N(c4cccc(-c5ccc6c(c5)oc5ccccc56)c4)c4cccc(-c5cccc6c5ccc5ccccc56)c4)c3)cc21 ; CC1(C)c2ccccc2-c2cc(-c3ccccc3N(c3cccc(-c4cccc5c4ccc4ccccc45)c3)c3ccccc3-c3cccc4oc5ccccc5c34)ccc21 ; CC1(C)c2ccccc2-c2cc(-c3ccccc3N(c3ccc(-c4ccc5c(c4)oc4ccccc45)cc3)c3cccc(-c4cccc5c4ccc4ccccc45)c3)ccc21]

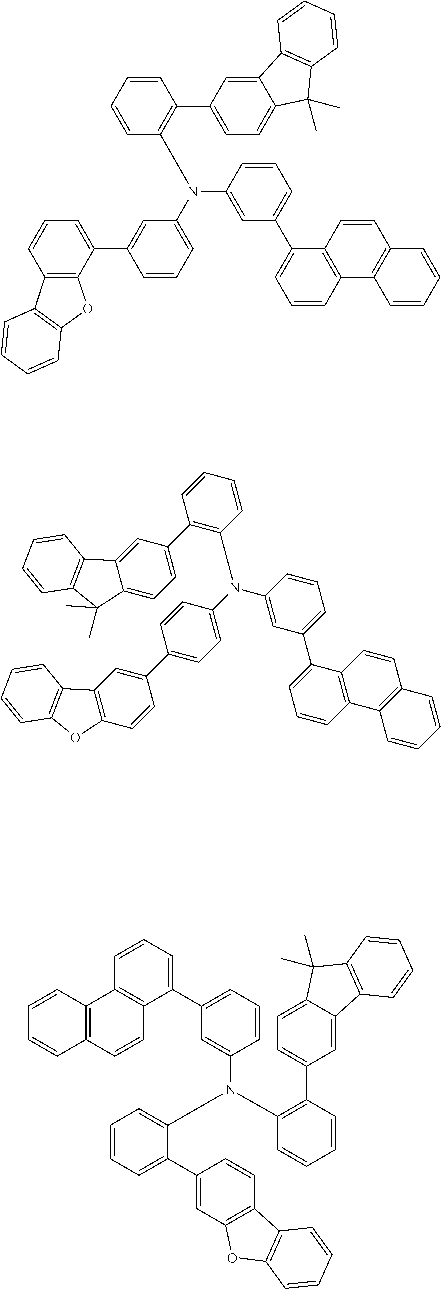 [SMILES: CC1(C)c2ccccc2-c2cc(-c3ccccc3N(c3cccc(-c4cccc5c4ccc4ccccc45)c3)c3cccc(-c4cccc5c4oc4ccccc45)c3)ccc21 ; CC1(C)c2ccccc2-c2cc(-c3ccccc3N(c3ccc(-c4ccc5oc6ccccc6c5c4)cc3)c3cccc(-c4cccc5c4ccc4ccccc45)c3)ccc21 ; CC1(C)c2ccccc2-c2cc(-c3ccccc3N(c3cccc(-c4cccc5c4ccc4ccccc45)c3)c3ccccc3-c3ccc4c(c3)oc3ccccc34)ccc21]

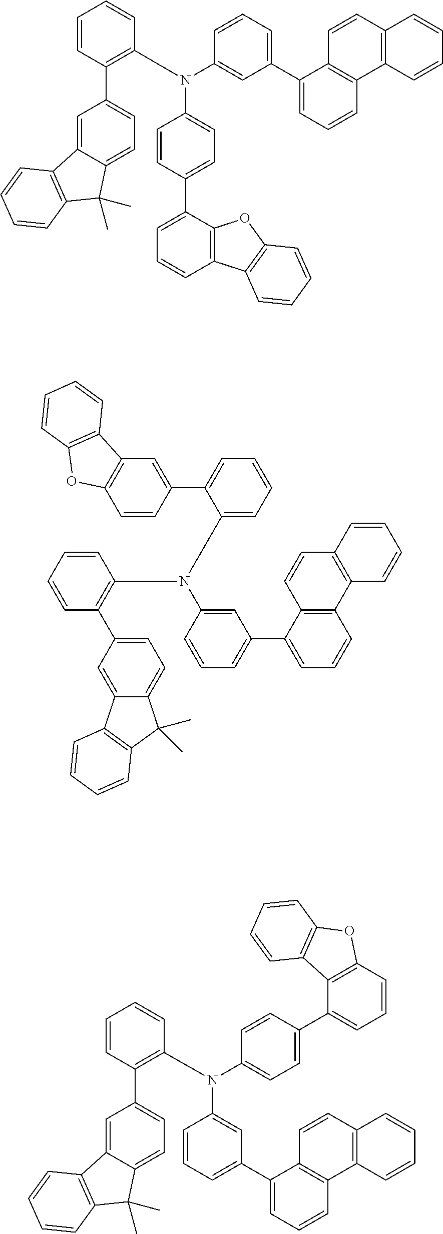 [SMILES: CC1(C)c2ccccc2-c2cc(-c3ccccc3N(c3ccc(-c4cccc5c4oc4ccccc45)cc3)c3cccc(-c4cccc5c4ccc4ccccc45)c3)ccc21 ; CC1(C)c2ccccc2-c2cc(-c3ccccc3N(c3cccc(-c4cccc5c4ccc4ccccc45)c3)c3ccccc3-c3ccc4oc5ccccc5c4c3)ccc21 ; CC1(C)c2ccccc2-c2cc(-c3ccccc3N(c3ccc(-c4cccc5oc6ccccc6c45)cc3)c3cccc(-c4cccc5c4ccc4ccccc45)c3)ccc21]

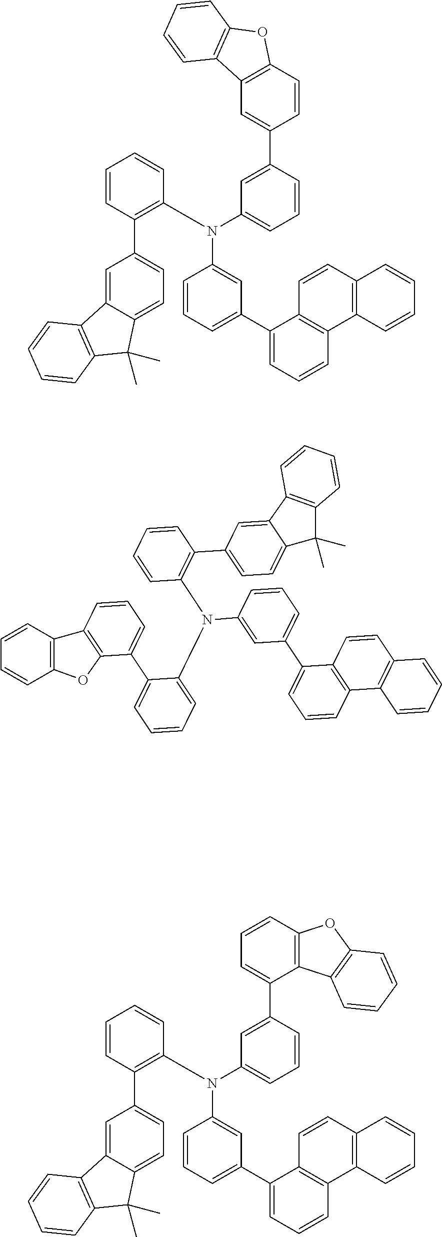 [SMILES: CC1(C)c2ccccc2-c2cc(-c3ccccc3N(c3cccc(-c4ccc5oc6ccccc6c5c4)c3)c3cccc(-c4cccc5c4ccc4ccccc45)c3)ccc21 ; CC1(C)c2ccccc2-c2cc(-c3ccccc3N(c3cccc(-c4cccc5c4ccc4ccccc45)c3)c3ccccc3-c3cccc4c3oc3ccccc34)ccc21 ; CC1(C)c2ccccc2-c2cc(-c3ccccc3N(c3cccc(-c4cccc5c4ccc4ccccc45)c3)c3cccc(-c4cccc5oc6ccccc6c45)c3)ccc21]

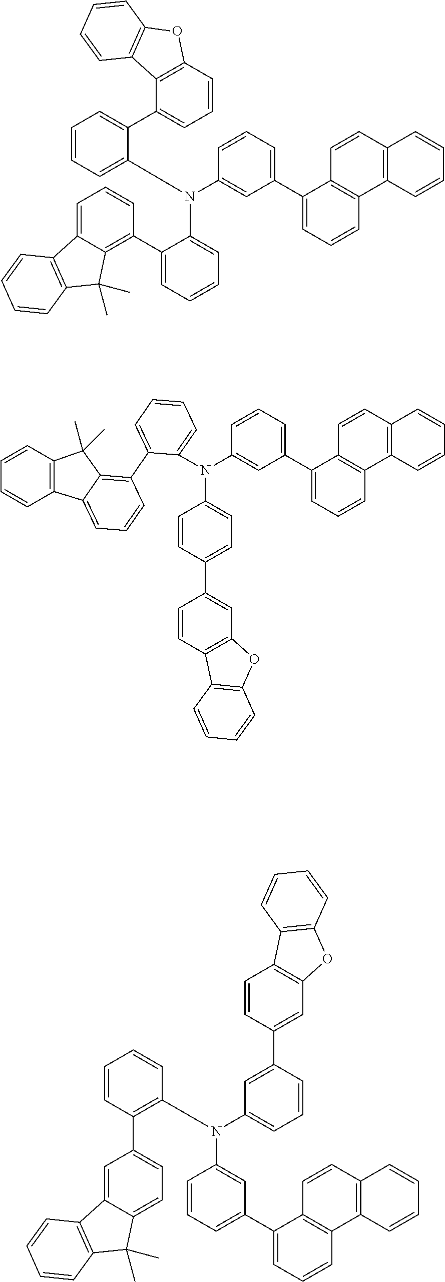 [SMILES: CC1(C)c2ccccc2-c2cccc(-c3ccccc3N(c3cccc(-c4cccc5c4ccc4ccccc45)c3)c3ccccc3-c3cccc4oc5ccccc5c34)c21 ; CC1(C)c2ccccc2-c2cccc(-c3ccccc3N(c3ccc(-c4ccc5c(c4)oc4ccccc45)cc3)c3cccc(-c4cccc5c4ccc4ccccc45)c3)c21 ; CC1(C)c2ccccc2-c2cc(-c3ccccc3N(c3cccc(-c4ccc5c(c4)oc4ccccc45)c3)c3cccc(-c4cccc5c4ccc4ccccc45)c3)ccc21]

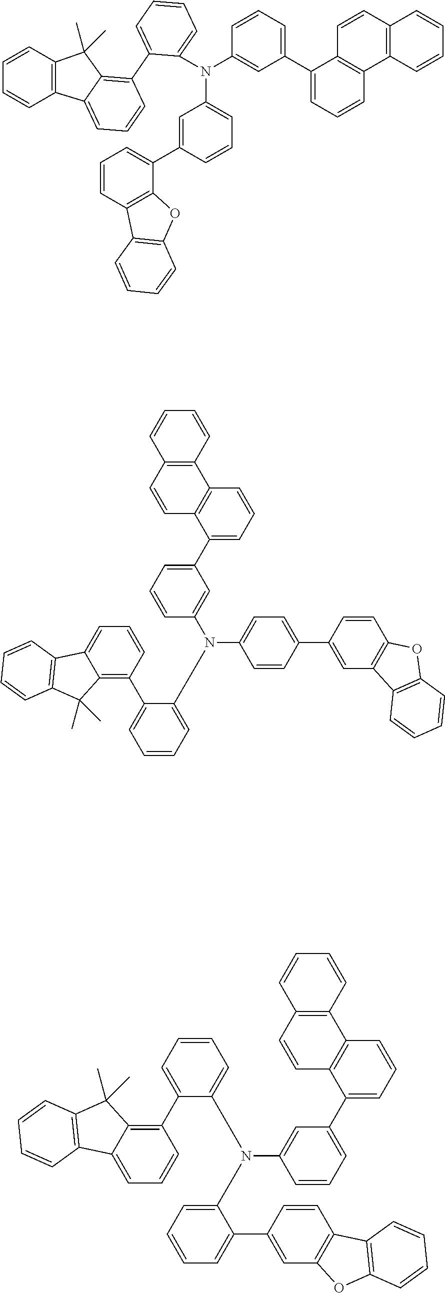 [SMILES: CC1(C)c2ccccc2-c2cccc(-c3ccccc3N(c3cccc(-c4cccc5c4ccc4ccccc45)c3)c3cccc(-c4cccc5c4oc4ccccc45)c3)c21 ; CC1(C)c2ccccc2-c2cccc(-c3ccccc3N(c3ccc(-c4ccc5oc6ccccc6c5c4)cc3)c3cccc(-c4cccc5c4ccc4ccccc45)c3)c21 ; CC1(C)c2ccccc2-c2cccc(-c3ccccc3N(c3cccc(-c4cccc5c4ccc4ccccc45)c3)c3ccccc3-c3ccc4c(c3)oc3ccccc34)c21]

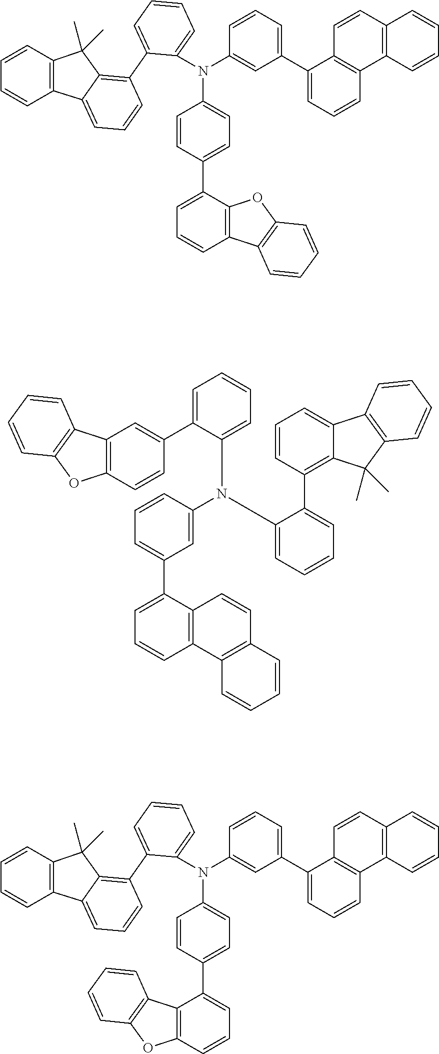 [SMILES: CC1(C)c2ccccc2-c2cccc(-c3ccccc3N(c3ccc(-c4cccc5c4oc4ccccc45)cc3)c3cccc(-c4cccc5c4ccc4ccccc45)c3)c21 ; CC1(C)c2ccccc2-c2cccc(-c3ccccc3N(c3cccc(-c4cccc5c4ccc4ccccc45)c3)c3ccccc3-c3ccc4oc5ccccc5c4c3)c21 ; CC1(C)c2ccccc2-c2cccc(-c3ccccc3N(c3ccc(-c4cccc5oc6ccccc6c45)cc3)c3cccc(-c4cccc5c4ccc4ccccc45)c3)c21]

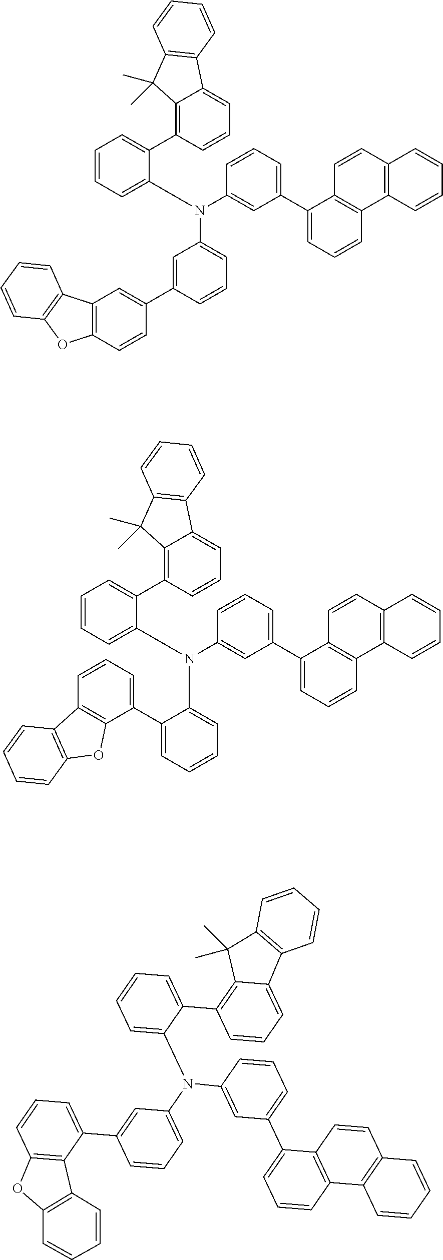 [SMILES: CC1(C)c2ccccc2-c2cccc(-c3ccccc3N(c3cccc(-c4ccc5oc6ccccc6c5c4)c3)c3cccc(-c4cccc5c4ccc4ccccc45)c3)c21 ; CC1(C)c2ccccc2-c2cccc(-c3ccccc3N(c3cccc(-c4cccc5c4ccc4ccccc45)c3)c3ccccc3-c3cccc4c3oc3ccccc34)c21 ; CC1(C)c2ccccc2-c2cccc(-c3ccccc3N(c3cccc(-c4cccc5c4ccc4ccccc45)c3)c3cccc(-c4cccc5oc6ccccc6c45)c3)c21]

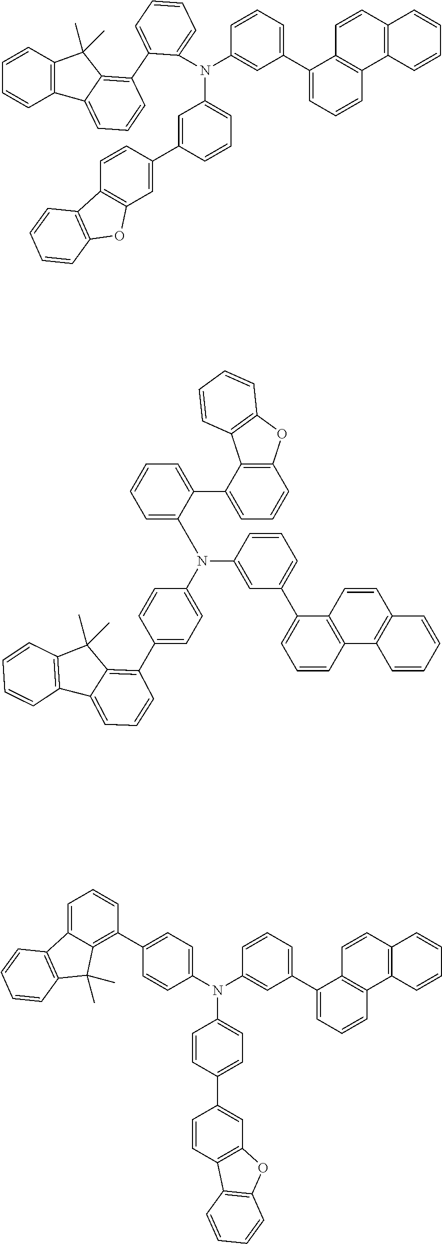 [SMILES: CC1(C)c2ccccc2-c2cccc(-c3ccccc3N(c3cccc(-c4ccc5c(c4)oc4ccccc45)c3)c3cccc(-c4cccc5c4ccc4ccccc45)c3)c21 ; CC1(C)c2ccccc2-c2cccc(-c3ccc(N(c4cccc(-c5cccc6c5ccc5ccccc56)c4)c4ccccc4-c4cccc5oc6ccccc6c45)cc3)c21 ; CC1(C)c2ccccc2-c2cccc(-c3ccc(N(c4ccc(-c5ccc6c(c5)oc5ccccc56)cc4)c4cccc(-c5cccc6c5ccc5ccccc56)c4)cc3)c21]

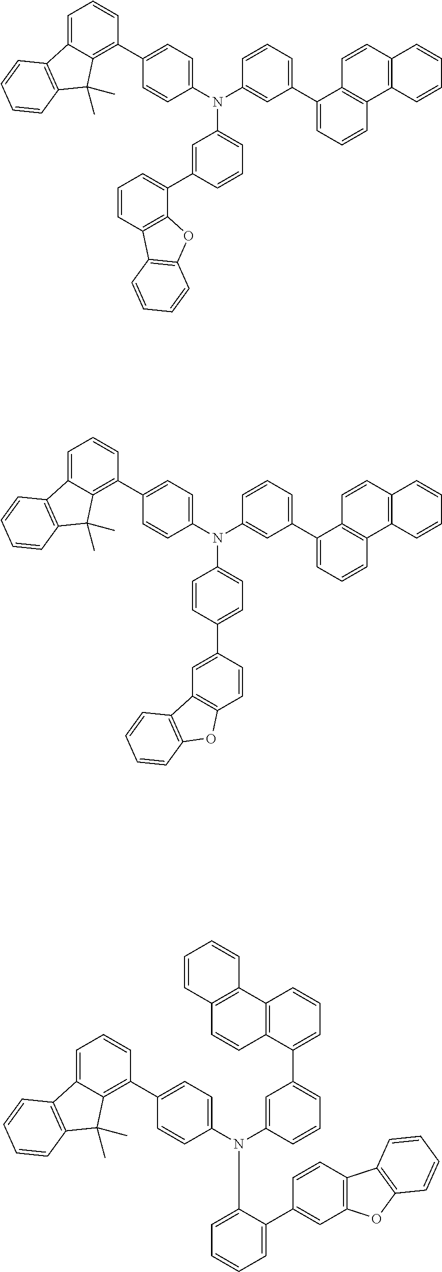 [SMILES: CC1(C)c2ccccc2-c2cccc(-c3ccc(N(c4cccc(-c5cccc6c5ccc5ccccc56)c4)c4cccc(-c5cccc6c5oc5ccccc56)c4)cc3)c21 ; CC1(C)c2ccccc2-c2cccc(-c3ccc(N(c4ccc(-c5ccc6oc7ccccc7c6c5)cc4)c4cccc(-c5cccc6c5ccc5ccccc56)c4)cc3)c21 ; CC1(C)c2ccccc2-c2cccc(-c3ccc(N(c4cccc(-c5cccc6c5ccc5ccccc56)c4)c4ccccc4-c4ccc5c(c4)oc4ccccc45)cc3)c21]

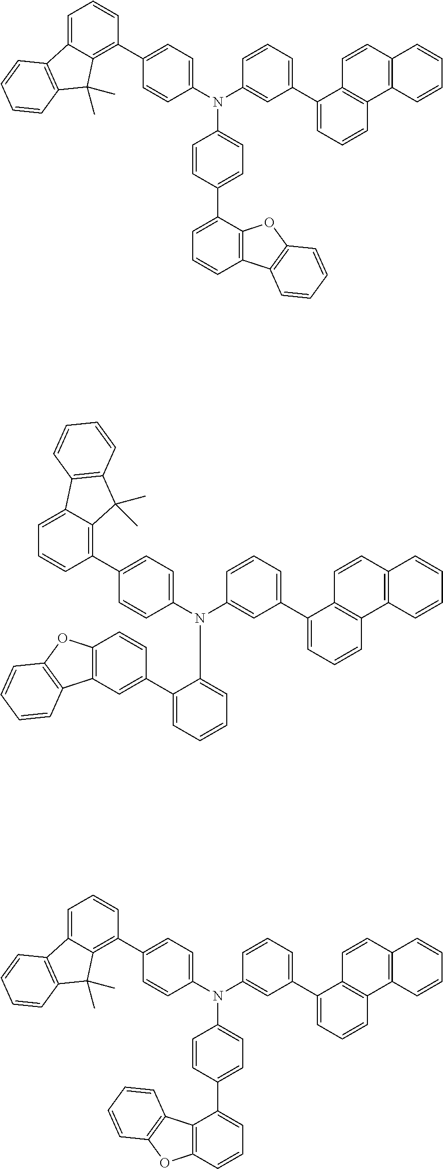 [SMILES: CC1(C)c2ccccc2-c2cccc(-c3ccc(N(c4ccc(-c5cccc6c5oc5ccccc56)cc4)c4cccc(-c5cccc6c5ccc5ccccc56)c4)cc3)c21 ; CC1(C)c2ccccc2-c2cccc(-c3ccc(N(c4cccc(-c5cccc6c5ccc5ccccc56)c4)c4ccccc4-c4ccc5oc6ccccc6c5c4)cc3)c21 ; CC1(C)c2ccccc2-c2cccc(-c3ccc(N(c4ccc(-c5cccc6oc7ccccc7c56)cc4)c4cccc(-c5cccc6c5ccc5ccccc56)c4)cc3)c21]

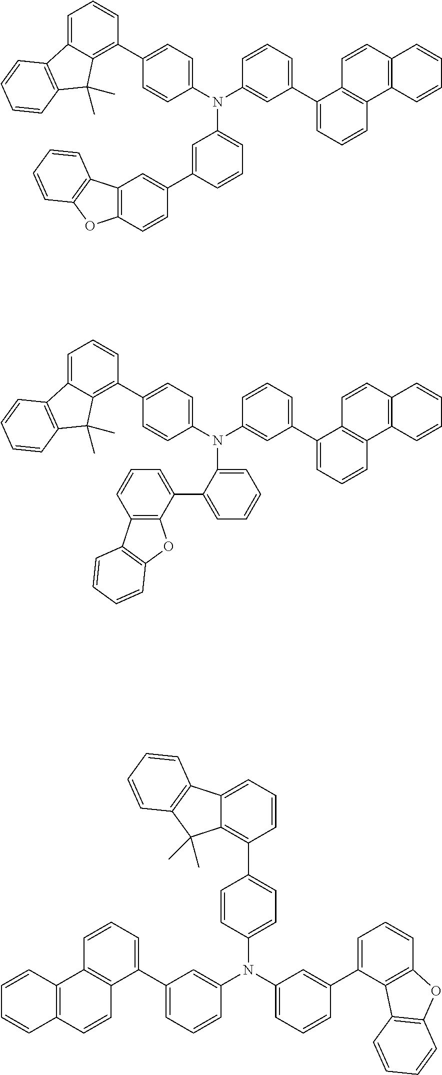 [SMILES: CC1(C)c2ccccc2-c2cccc(-c3ccc(N(c4cccc(-c5ccc6oc7ccccc7c6c5)c4)c4cccc(-c5cccc6c5ccc5ccccc56)c4)cc3)c21 ; CC1(C)c2ccccc2-c2cccc(-c3ccc(N(c4cccc(-c5cccc6c5ccc5ccccc56)c4)c4ccccc4-c4cccc5c4oc4ccccc45)cc3)c21 ; CC1(C)c2ccccc2-c2cccc(-c3ccc(N(c4cccc(-c5cccc6c5ccc5ccccc56)c4)c4cccc(-c5cccc6oc7ccccc7c56)c4)cc3)c21]

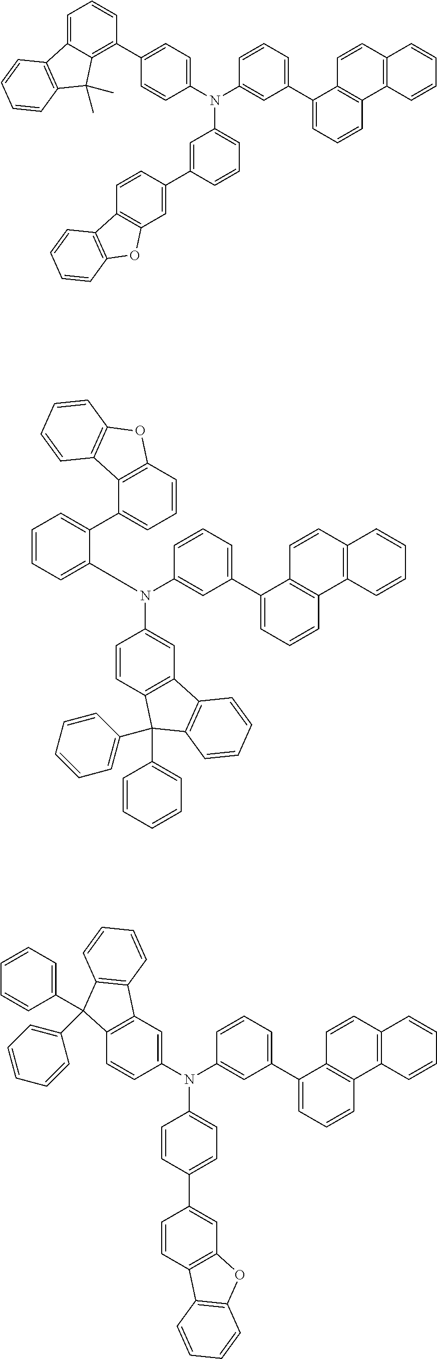 [SMILES: CC1(C)c2ccccc2-c2cccc(-c3ccc(N(c4cccc(-c5ccc6c(c5)oc5ccccc56)c4)c4cccc(-c5cccc6c5ccc5ccccc56)c4)cc3)c21 ; c1ccc(C2(c3ccccc3)c3ccccc3-c3cc(N(c4cccc(-c5cccc6c5ccc5ccccc56)c4)c4ccccc4-c4cccc5oc6ccccc6c45)ccc32)cc1 ; c1ccc(C2(c3ccccc3)c3ccccc3-c3cc(N(c4ccc(-c5ccc6c(c5)oc5ccccc56)cc4)c4cccc(-c5cccc6c5ccc5ccccc56)c4)ccc32)cc1]

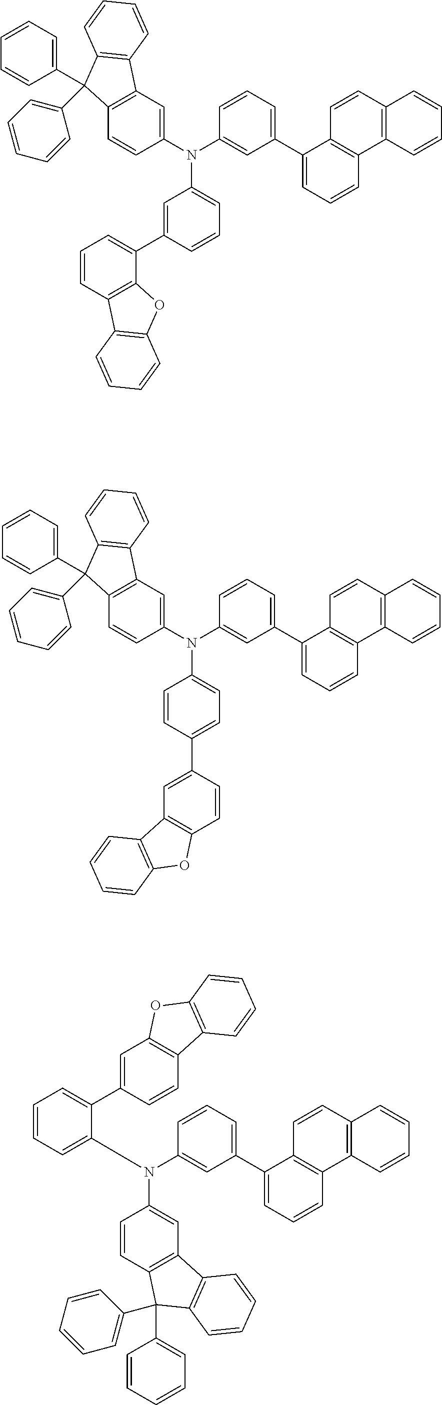 [SMILES: c1ccc(C2(c3ccccc3)c3ccccc3-c3cc(N(c4cccc(-c5cccc6c5ccc5ccccc56)c4)c4cccc(-c5cccc6c5oc5ccccc56)c4)ccc32)cc1 ; c1ccc(C2(c3ccccc3)c3ccccc3-c3cc(N(c4ccc(-c5ccc6oc7ccccc7c6c5)cc4)c4cccc(-c5cccc6c5ccc5ccccc56)c4)ccc32)cc1 ; c1ccc(C2(c3ccccc3)c3ccccc3-c3cc(N(c4cccc(-c5cccc6c5ccc5ccccc56)c4)c4ccccc4-c4ccc5c(c4)oc4ccccc45)ccc32)cc1]

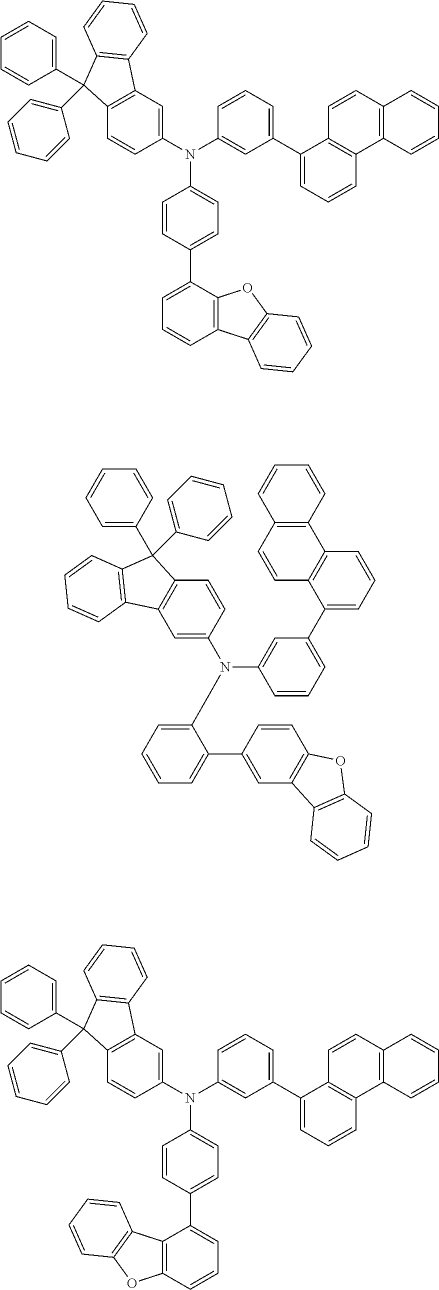 [SMILES: c1ccc(C2(c3ccccc3)c3ccccc3-c3cc(N(c4ccc(-c5cccc6c5oc5ccccc56)cc4)c4cccc(-c5cccc6c5ccc5ccccc56)c4)ccc32)cc1 ; c1ccc(C2(c3ccccc3)c3ccccc3-c3cc(N(c4cccc(-c5cccc6c5ccc5ccccc56)c4)c4ccccc4-c4ccc5oc6ccccc6c5c4)ccc32)cc1 ; c1ccc(C2(c3ccccc3)c3ccccc3-c3cc(N(c4ccc(-c5cccc6oc7ccccc7c56)cc4)c4cccc(-c5cccc6c5ccc5ccccc56)c4)ccc32)cc1]

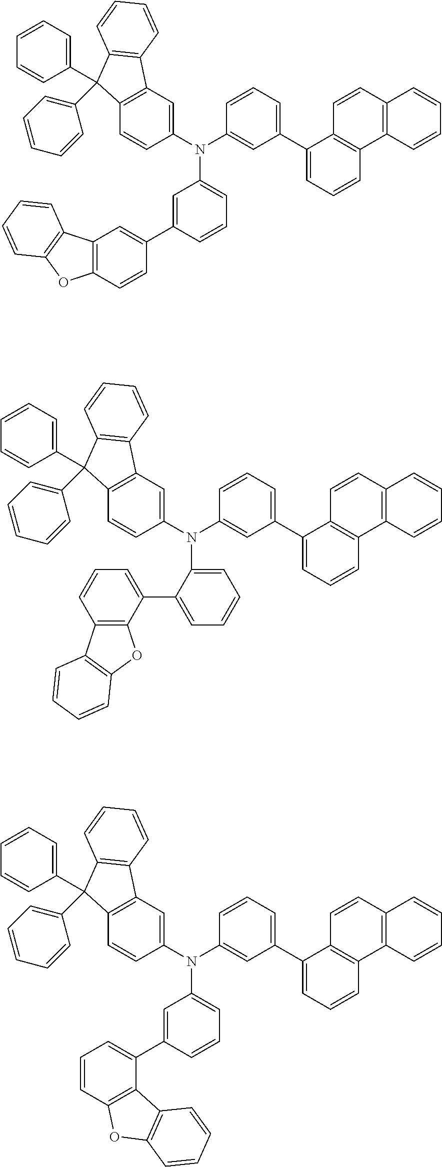 [SMILES: c1ccc(C2(c3ccccc3)c3ccccc3-c3cc(N(c4cccc(-c5ccc6oc7ccccc7c6c5)c4)c4cccc(-c5cccc6c5ccc5ccccc56)c4)ccc32)cc1 ; c1ccc(C2(c3ccccc3)c3ccccc3-c3cc(N(c4cccc(-c5cccc6c5ccc5ccccc56)c4)c4ccccc4-c4cccc5c4oc4ccccc45)ccc32)cc1 ; c1ccc(C2(c3ccccc3)c3ccccc3-c3cc(N(c4cccc(-c5cccc6c5ccc5ccccc56)c4)c4cccc(-c5cccc6oc7ccccc7c56)c4)ccc32)cc1]

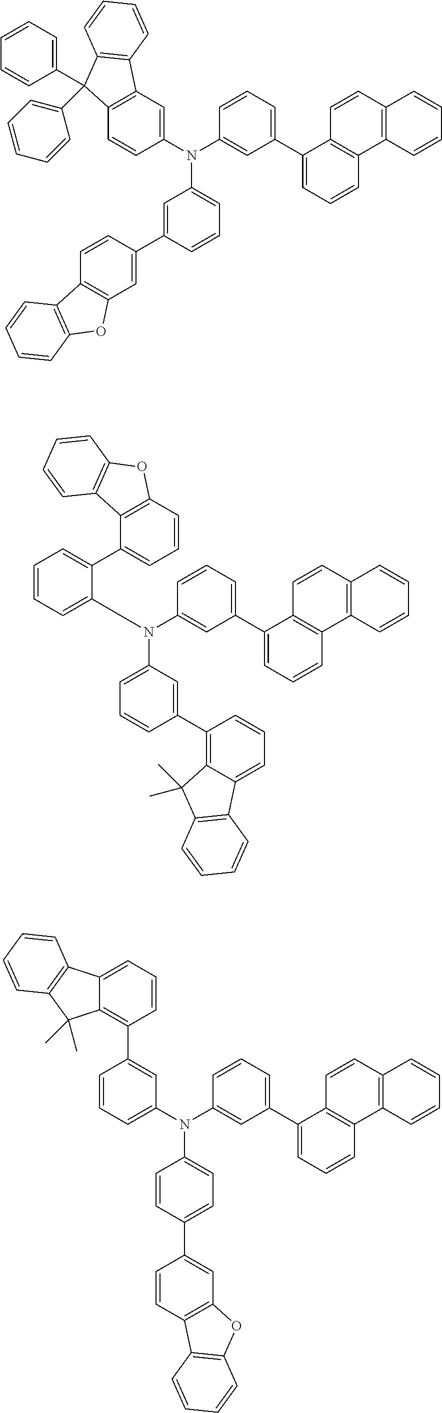 [SMILES: c1ccc(C2(c3ccccc3)c3ccccc3-c3cc(N(c4cccc(-c5ccc6c(c5)oc5ccccc56)c4)c4cccc(-c5cccc6c5ccc5ccccc56)c4)ccc32)cc1 ; CC1(C)c2ccccc2-c2cccc(-c3cccc(N(c4cccc(-c5cccc6c5ccc5ccccc56)c4)c4ccccc4-c4cccc5oc6ccccc6c45)c3)c21 ; CC1(C)c2ccccc2-c2cccc(-c3cccc(N(c4ccc(-c5ccc6c(c5)oc5ccccc56)cc4)c4cccc(-c5cccc6c5ccc5ccccc56)c4)c3)c21]

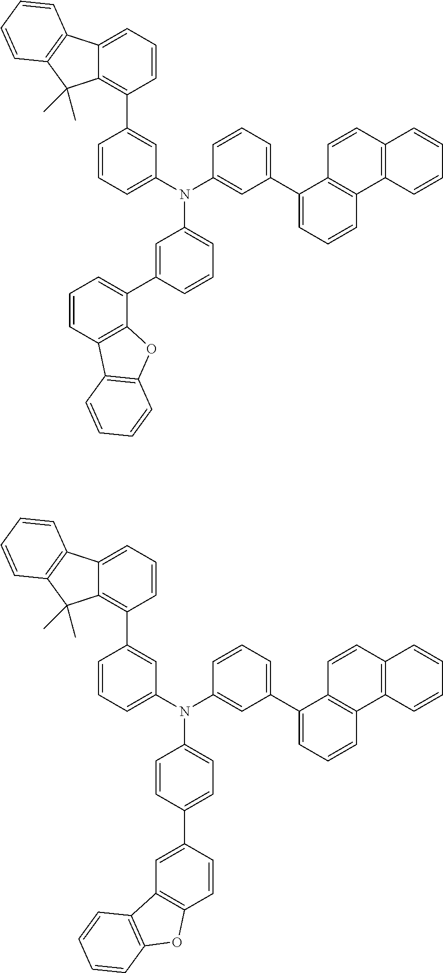 [SMILES: CC1(C)c2ccccc2-c2cccc(-c3cccc(N(c4cccc(-c5cccc6c5ccc5ccccc56)c4)c4cccc(-c5cccc6c5oc5ccccc56)c4)c3)c21 ; CC1(C)c2ccccc2-c2cccc(-c3cccc(N(c4ccc(-c5ccc6oc7ccccc7c6c5)cc4)c4cccc(-c5cccc6c5ccc5ccccc56)c4)c3)c21]

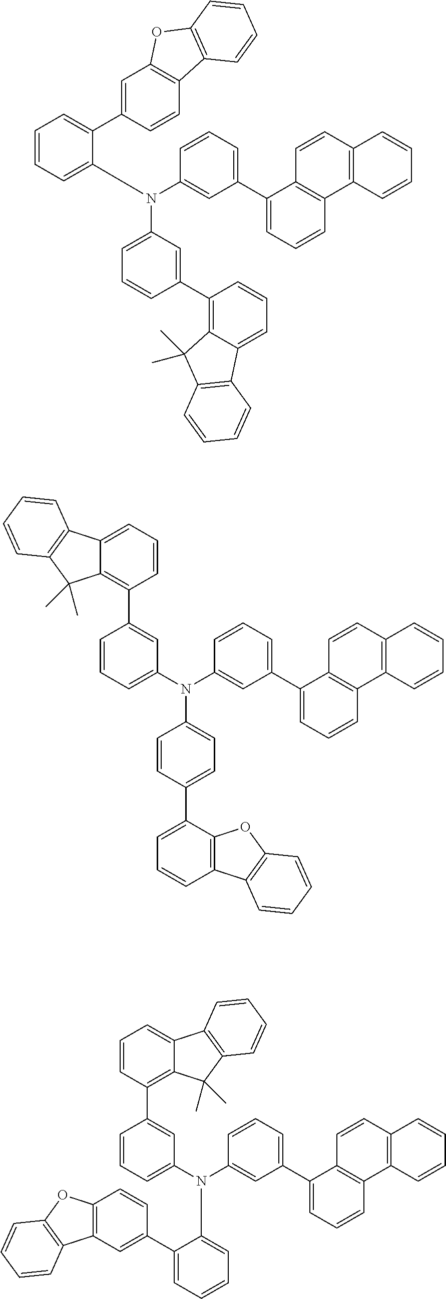 [SMILES: CC1(C)c2ccccc2-c2cccc(-c3cccc(N(c4cccc(-c5cccc6c5ccc5ccccc56)c4)c4ccccc4-c4ccc5c(c4)oc4ccccc45)c3)c21 ; CC1(C)c2ccccc2-c2cccc(-c3cccc(N(c4ccc(-c5cccc6c5oc5ccccc56)cc4)c4cccc(-c5cccc6c5ccc5ccccc56)c4)c3)c21 ; CC1(C)c2ccccc2-c2cccc(-c3cccc(N(c4cccc(-c5cccc6c5ccc5ccccc56)c4)c4ccccc4-c4ccc5oc6ccccc6c5c4)c3)c21]

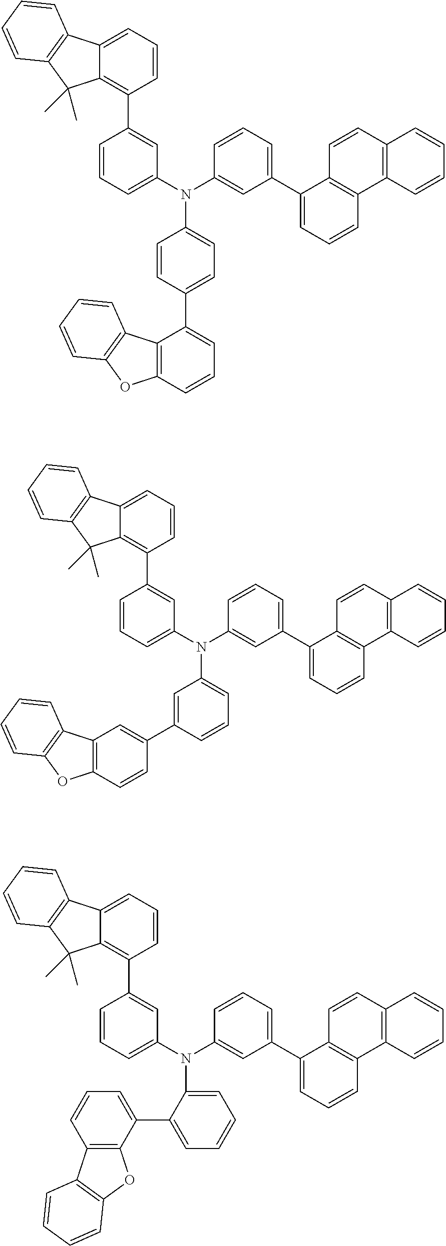 [SMILES: CC1(C)c2ccccc2-c2cccc(-c3cccc(N(c4ccc(-c5cccc6oc7ccccc7c56)cc4)c4cccc(-c5cccc6c5ccc5ccccc56)c4)c3)c21 ; CC1(C)c2ccccc2-c2cccc(-c3cccc(N(c4cccc(-c5ccc6oc7ccccc7c6c5)c4)c4cccc(-c5cccc6c5ccc5ccccc56)c4)c3)c21 ; CC1(C)c2ccccc2-c2cccc(-c3cccc(N(c4cccc(-c5cccc6c5ccc5ccccc56)c4)c4ccccc4-c4cccc5c4oc4ccccc45)c3)c21]

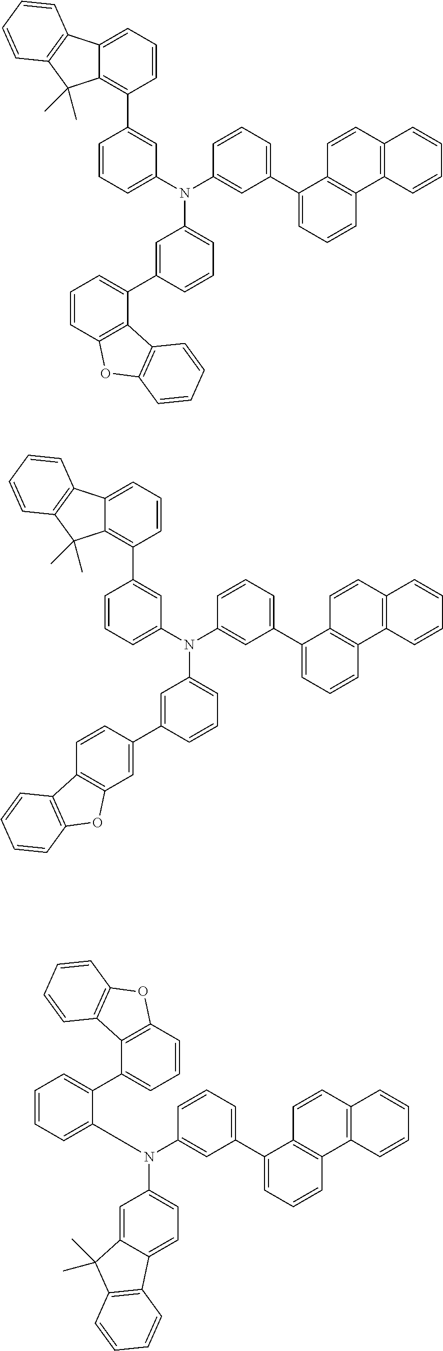 [SMILES: CC1(C)c2ccccc2-c2cccc(-c3cccc(N(c4cccc(-c5cccc6c5ccc5ccccc56)c4)c4cccc(-c5cccc6oc7ccccc7c56)c4)c3)c21 ; CC1(C)c2ccccc2-c2cccc(-c3cccc(N(c4cccc(-c5ccc6c(c5)oc5ccccc56)c4)c4cccc(-c5cccc6c5ccc5ccccc56)c4)c3)c21 ; CC1(C)c2ccccc2-c2ccc(N(c3cccc(-c4cccc5c4ccc4ccccc45)c3)c3ccccc3-c3cccc4oc5ccccc5c34)cc21]

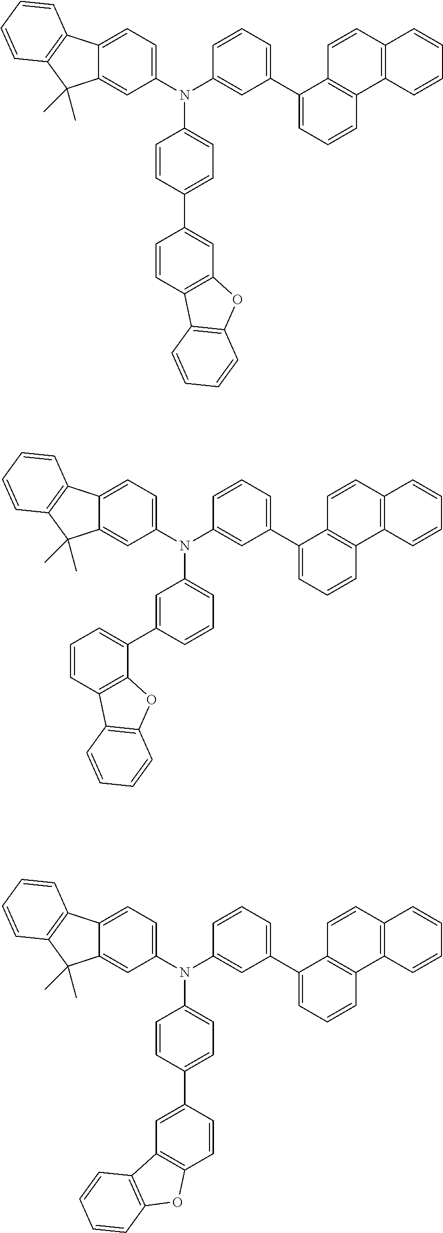 [SMILES: CC1(C)c2ccccc2-c2ccc(N(c3ccc(-c4ccc5c(c4)oc4ccccc45)cc3)c3cccc(-c4cccc5c4ccc4ccccc45)c3)cc21 ; CC1(C)c2ccccc2-c2ccc(N(c3cccc(-c4cccc5c4ccc4ccccc45)c3)c3cccc(-c4cccc5c4oc4ccccc45)c3)cc21 ; CC1(C)c2ccccc2-c2ccc(N(c3ccc(-c4ccc5oc6ccccc6c5c4)cc3)c3cccc(-c4cccc5c4ccc4ccccc45)c3)cc21]

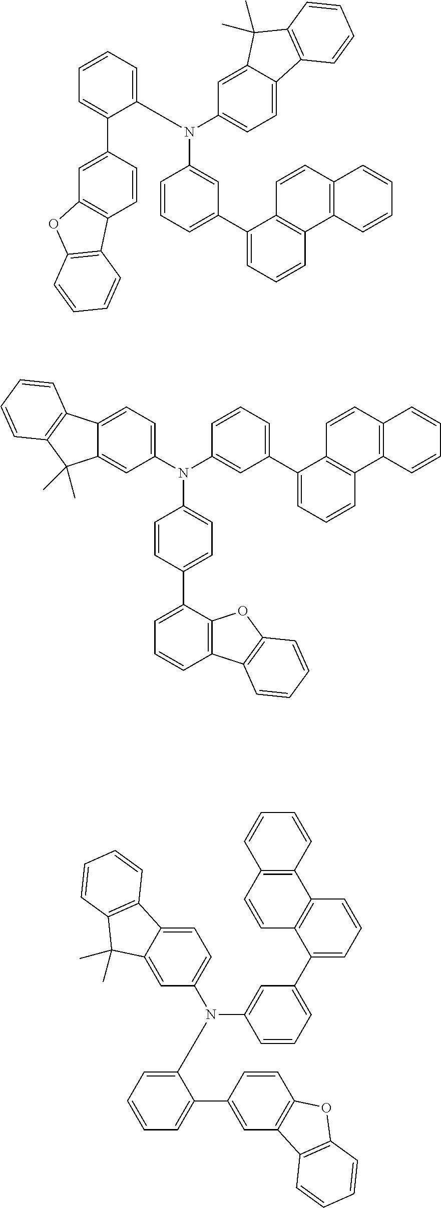 [SMILES: CC1(C)c2ccccc2-c2ccc(N(c3cccc(-c4cccc5c4ccc4ccccc45)c3)c3ccccc3-c3ccc4c(c3)oc3ccccc34)cc21 ; CC1(C)c2ccccc2-c2ccc(N(c3ccc(-c4cccc5c4oc4ccccc45)cc3)c3cccc(-c4cccc5c4ccc4ccccc45)c3)cc21 ; CC1(C)c2ccccc2-c2ccc(N(c3cccc(-c4cccc5c4ccc4ccccc45)c3)c3ccccc3-c3ccc4oc5ccccc5c4c3)cc21]

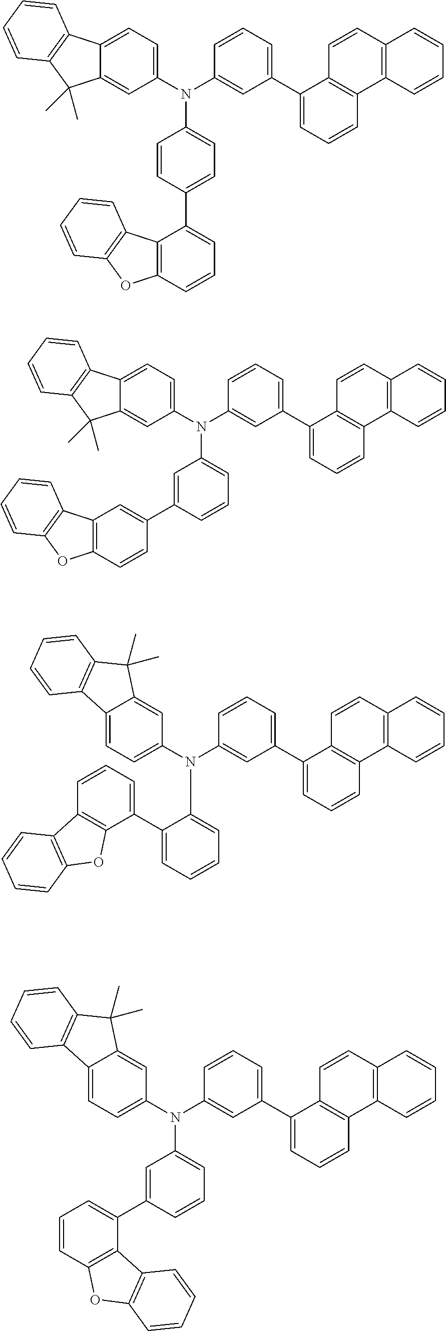 [SMILES: CC1(C)c2ccccc2-c2ccc(N(c3ccc(-c4cccc5oc6ccccc6c45)cc3)c3cccc(-c4cccc5c4ccc4ccccc45)c3)cc21 ; CC1(C)c2ccccc2-c2ccc(N(c3cccc(-c4ccc5oc6ccccc6c5c4)c3)c3cccc(-c4cccc5c4ccc4ccccc45)c3)cc21 ; CC1(C)c2ccccc2-c2ccc(N(c3cccc(-c4cccc5c4ccc4ccccc45)c3)c3ccccc3-c3cccc4c3oc3ccccc34)cc21 ; CC1(C)c2ccccc2-c2ccc(N(c3cccc(-c4cccc5c4ccc4ccccc45)c3)c3cccc(-c4cccc5oc6ccccc6c45)c3)cc21]

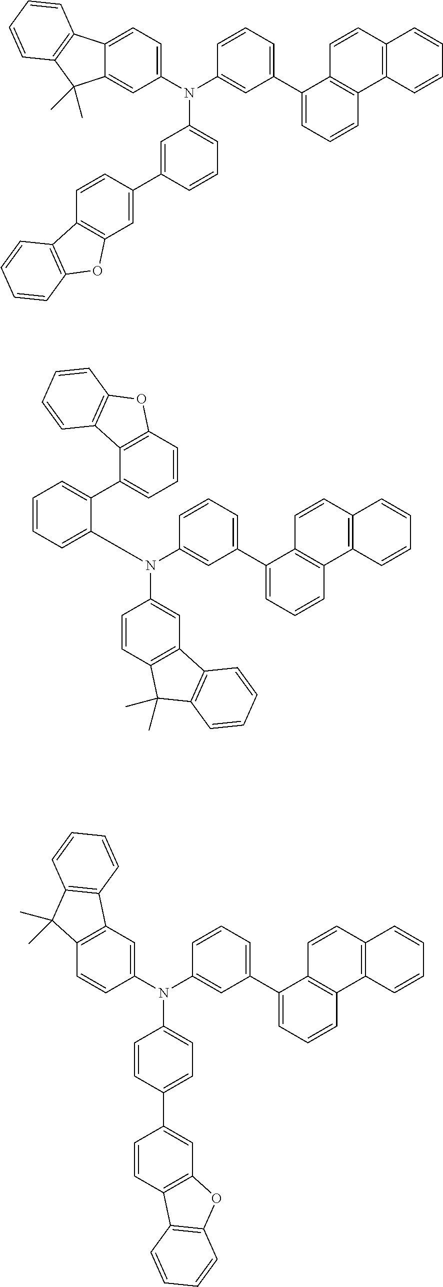 [SMILES: CC1(C)c2ccccc2-c2ccc(N(c3cccc(-c4ccc5c(c4)oc4ccccc45)c3)c3cccc(-c4cccc5c4ccc4ccccc45)c3)cc21 ; CC1(C)c2ccccc2-c2cc(N(c3cccc(-c4cccc5c4ccc4ccccc45)c3)c3ccccc3-c3cccc4oc5ccccc5c34)ccc21 ; CC1(C)c2ccccc2-c2cc(N(c3ccc(-c4ccc5c(c4)oc4ccccc45)cc3)c3cccc(-c4cccc5c4ccc4ccccc45)c3)ccc21]

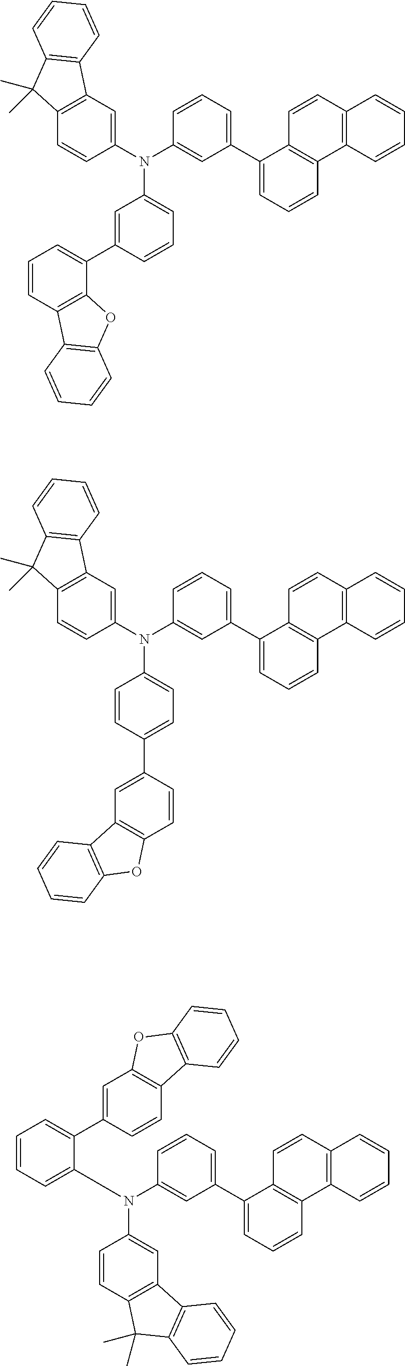 [SMILES: CC1(C)c2ccccc2-c2cc(N(c3cccc(-c4cccc5c4ccc4ccccc45)c3)c3cccc(-c4cccc5c4oc4ccccc45)c3)ccc21 ; CC1(C)c2ccccc2-c2cc(N(c3ccc(-c4ccc5oc6ccccc6c5c4)cc3)c3cccc(-c4cccc5c4ccc4ccccc45)c3)ccc21 ; CC1(C)c2ccccc2-c2cc(N(c3cccc(-c4cccc5c4ccc4ccccc45)c3)c3ccccc3-c3ccc4c(c3)oc3ccccc34)ccc21]

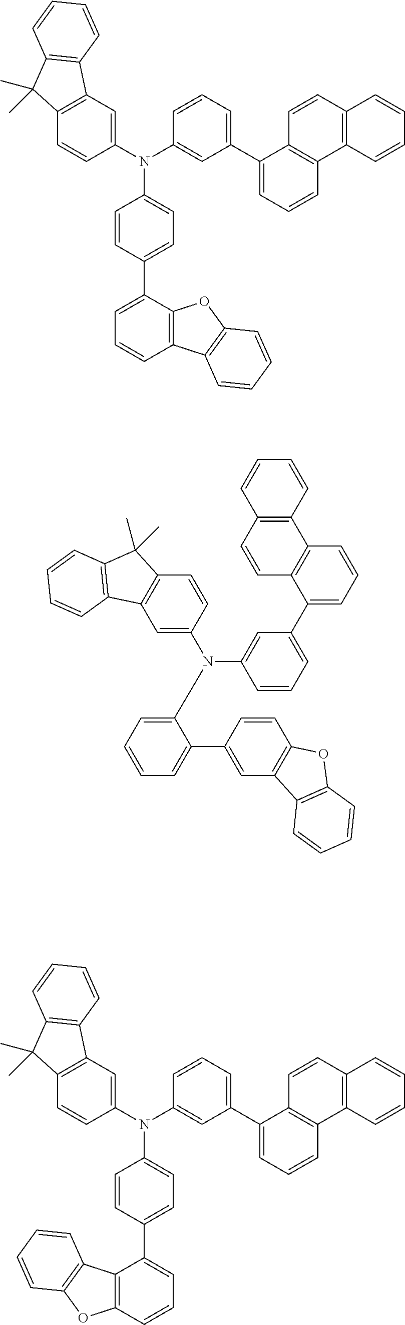 [SMILES: CC1(C)c2ccccc2-c2cc(N(c3ccc(-c4cccc5c4oc4ccccc45)cc3)c3cccc(-c4cccc5c4ccc4ccccc45)c3)ccc21 ; CC1(C)c2ccccc2-c2cc(N(c3cccc(-c4cccc5c4ccc4ccccc45)c3)c3ccccc3-c3ccc4oc5ccccc5c4c3)ccc21 ; CC1(C)c2ccccc2-c2cc(N(c3ccc(-c4cccc5oc6ccccc6c45)cc3)c3cccc(-c4cccc5c4ccc4ccccc45)c3)ccc21]

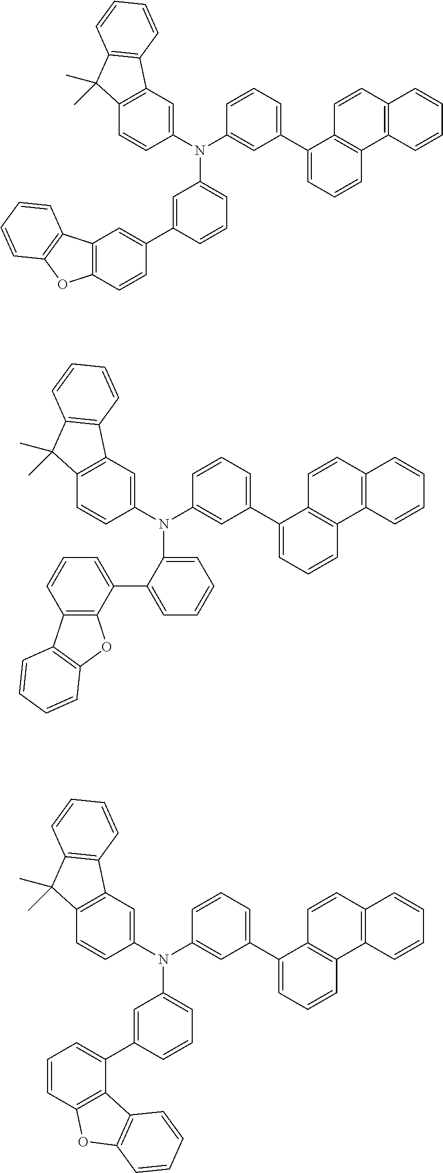 [SMILES: CC1(C)c2ccccc2-c2cc(N(c3cccc(-c4ccc5oc6ccccc6c5c4)c3)c3cccc(-c4cccc5c4ccc4ccccc45)c3)ccc21 ; CC1(C)c2ccccc2-c2cc(N(c3cccc(-c4cccc5c4ccc4ccccc45)c3)c3ccccc3-c3cccc4c3oc3ccccc34)ccc21 ; CC1(C)c2ccccc2-c2cc(N(c3cccc(-c4cccc5c4ccc4ccccc45)c3)c3cccc(-c4cccc5oc6ccccc6c45)c3)ccc21]

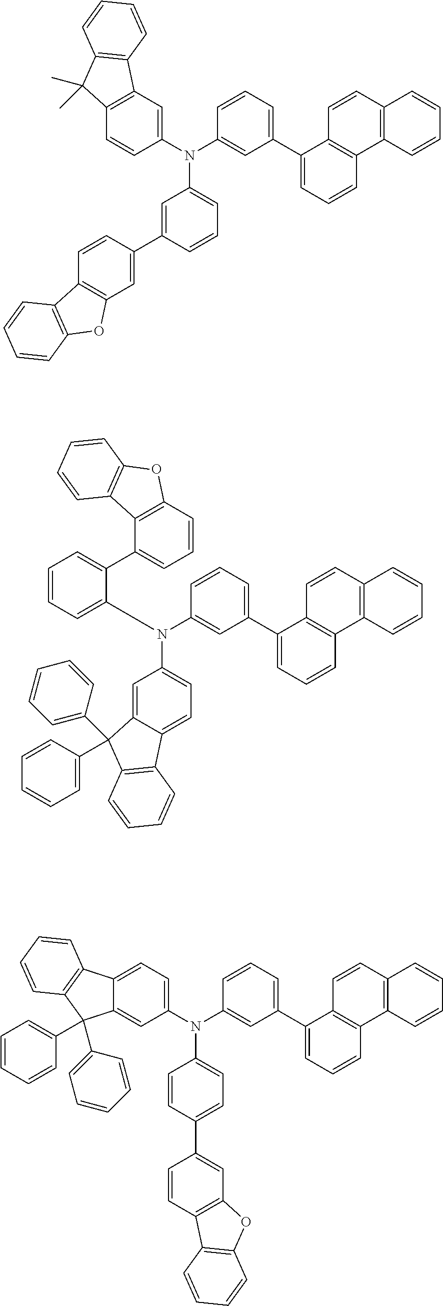 [SMILES: CC1(C)c2ccccc2-c2cc(N(c3cccc(-c4ccc5c(c4)oc4ccccc45)c3)c3cccc(-c4cccc5c4ccc4ccccc45)c3)ccc21 ; c1ccc(C2(c3ccccc3)c3ccccc3-c3ccc(N(c4cccc(-c5cccc6c5ccc5ccccc56)c4)c4ccccc4-c4cccc5oc6ccccc6c45)cc32)cc1 ; c1ccc(C2(c3ccccc3)c3ccccc3-c3ccc(N(c4ccc(-c5ccc6c(c5)oc5ccccc56)cc4)c4cccc(-c5cccc6c5ccc5ccccc56)c4)cc32)cc1]

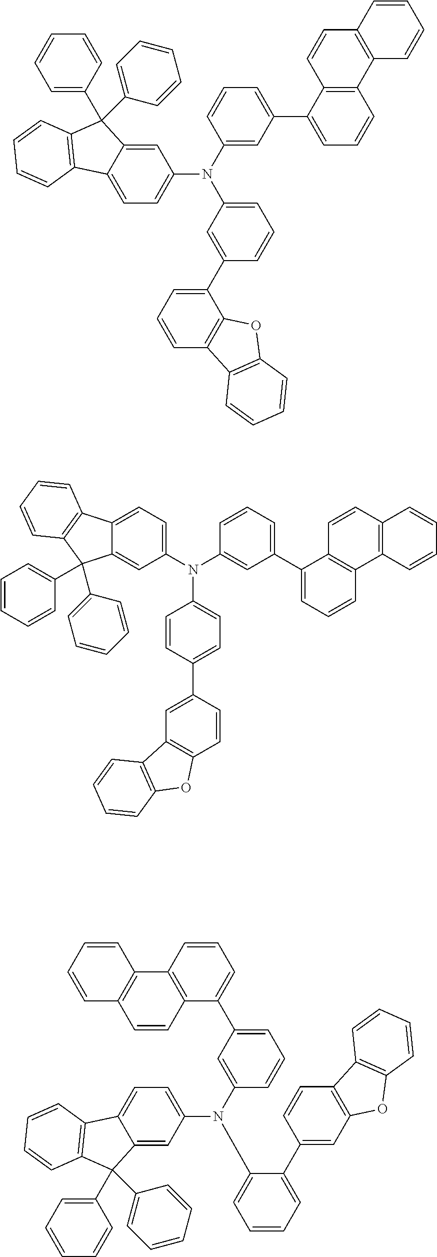 [SMILES: c1ccc(C2(c3ccccc3)c3ccccc3-c3ccc(N(c4cccc(-c5cccc6c5ccc5ccccc56)c4)c4cccc(-c5cccc6c5oc5ccccc56)c4)cc32)cc1 ; c1ccc(C2(c3ccccc3)c3ccccc3-c3ccc(N(c4ccc(-c5ccc6oc7ccccc7c6c5)cc4)c4cccc(-c5cccc6c5ccc5ccccc56)c4)cc32)cc1 ; c1ccc(C2(c3ccccc3)c3ccccc3-c3ccc(N(c4cccc(-c5cccc6c5ccc5ccccc56)c4)c4ccccc4-c4ccc5c(c4)oc4ccccc45)cc32)cc1]

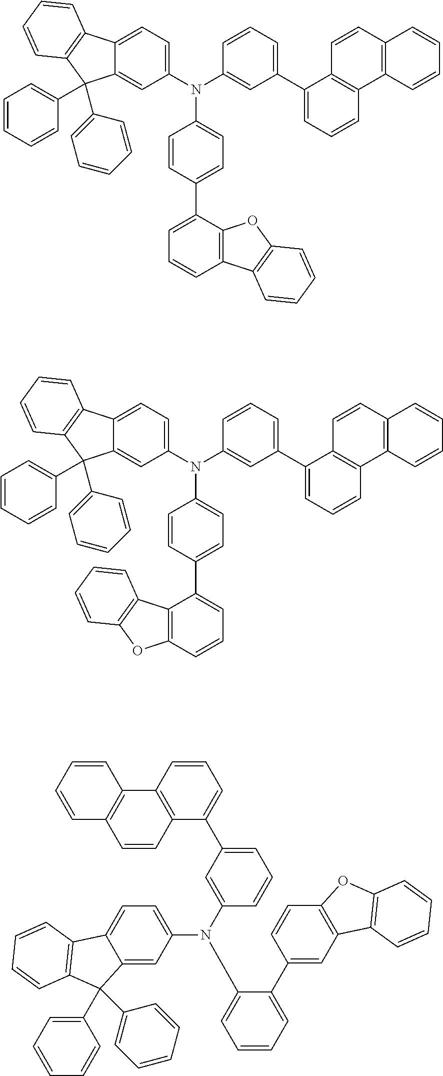 [SMILES: c1ccc(C2(c3ccccc3)c3ccccc3-c3ccc(N(c4ccc(-c5cccc6c5oc5ccccc56)cc4)c4cccc(-c5cccc6c5ccc5ccccc56)c4)cc32)cc1 ; c1ccc(C2(c3ccccc3)c3ccccc3-c3ccc(N(c4ccc(-c5cccc6oc7ccccc7c56)cc4)c4cccc(-c5cccc6c5ccc5ccccc56)c4)cc32)cc1 ; c1ccc(C2(c3ccccc3)c3ccccc3-c3ccc(N(c4cccc(-c5cccc6c5ccc5ccccc56)c4)c4ccccc4-c4ccc5oc6ccccc6c5c4)cc32)cc1]

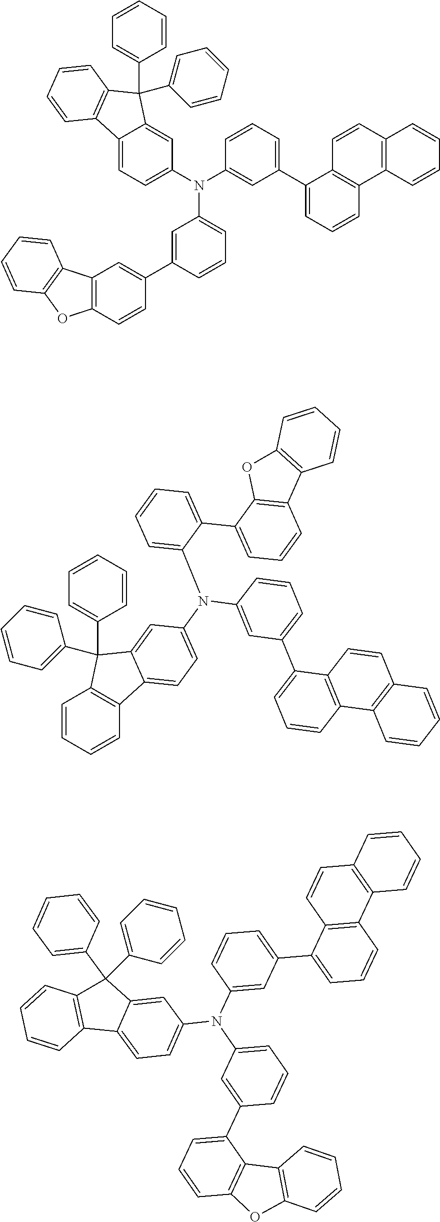 [SMILES: c1ccc(C2(c3ccccc3)c3ccccc3-c3ccc(N(c4cccc(-c5ccc6oc7ccccc7c6c5)c4)c4cccc(-c5cccc6c5ccc5ccccc56)c4)cc32)cc1 ; c1ccc(C2(c3ccccc3)c3ccccc3-c3ccc(N(c4cccc(-c5cccc6c5ccc5ccccc56)c4)c4ccccc4-c4cccc5c4oc4ccccc45)cc32)cc1 ; c1ccc(C2(c3ccccc3)c3ccccc3-c3ccc(N(c4cccc(-c5cccc6c5ccc5ccccc56)c4)c4cccc(-c5cccc6oc7ccccc7c56)c4)cc32)cc1]

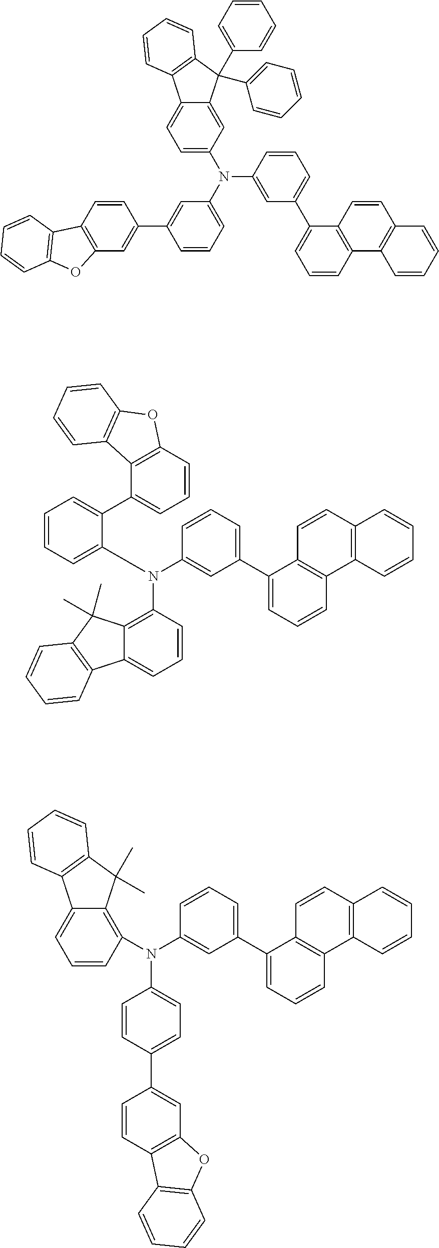 [SMILES: c1ccc(C2(c3ccccc3)c3ccccc3-c3ccc(N(c4cccc(-c5ccc6c(c5)oc5ccccc56)c4)c4cccc(-c5cccc6c5ccc5ccccc56)c4)cc32)cc1 ; CC1(C)c2ccccc2-c2cccc(N(c3cccc(-c4cccc5c4ccc4ccccc45)c3)c3ccccc3-c3cccc4oc5ccccc5c34)c21 ; CC1(C)c2ccccc2-c2cccc(N(c3ccc(-c4ccc5c(c4)oc4ccccc45)cc3)c3cccc(-c4cccc5c4ccc4ccccc45)c3)c21]

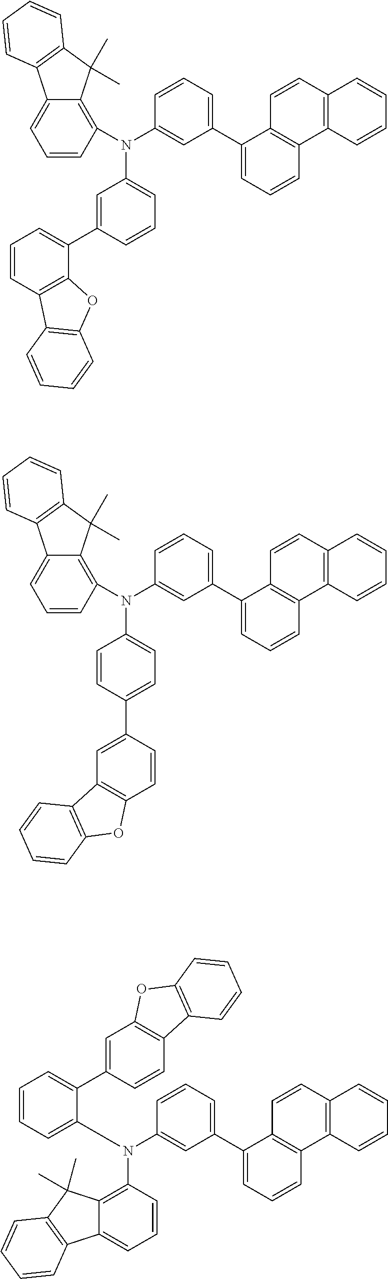 [SMILES: CC1(C)c2ccccc2-c2cccc(N(c3cccc(-c4cccc5c4ccc4ccccc45)c3)c3cccc(-c4cccc5c4oc4ccccc45)c3)c21 ; CC1(C)c2ccccc2-c2cccc(N(c3ccc(-c4ccc5oc6ccccc6c5c4)cc3)c3cccc(-c4cccc5c4ccc4ccccc45)c3)c21 ; CC1(C)c2ccccc2-c2cccc(N(c3cccc(-c4cccc5c4ccc4ccccc45)c3)c3ccccc3-c3ccc4c(c3)oc3ccccc34)c21]

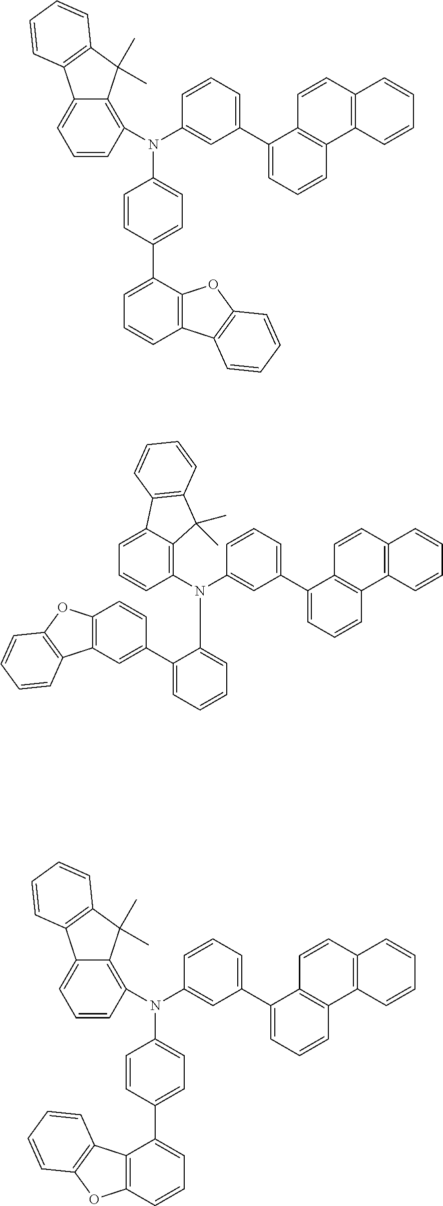 [SMILES: CC1(C)c2ccccc2-c2cccc(N(c3ccc(-c4cccc5c4oc4ccccc45)cc3)c3cccc(-c4cccc5c4ccc4ccccc45)c3)c21 ; CC1(C)c2ccccc2-c2cccc(N(c3cccc(-c4cccc5c4ccc4ccccc45)c3)c3ccccc3-c3ccc4oc5ccccc5c4c3)c21 ; CC1(C)c2ccccc2-c2cccc(N(c3ccc(-c4cccc5oc6ccccc6c45)cc3)c3cccc(-c4cccc5c4ccc4ccccc45)c3)c21]

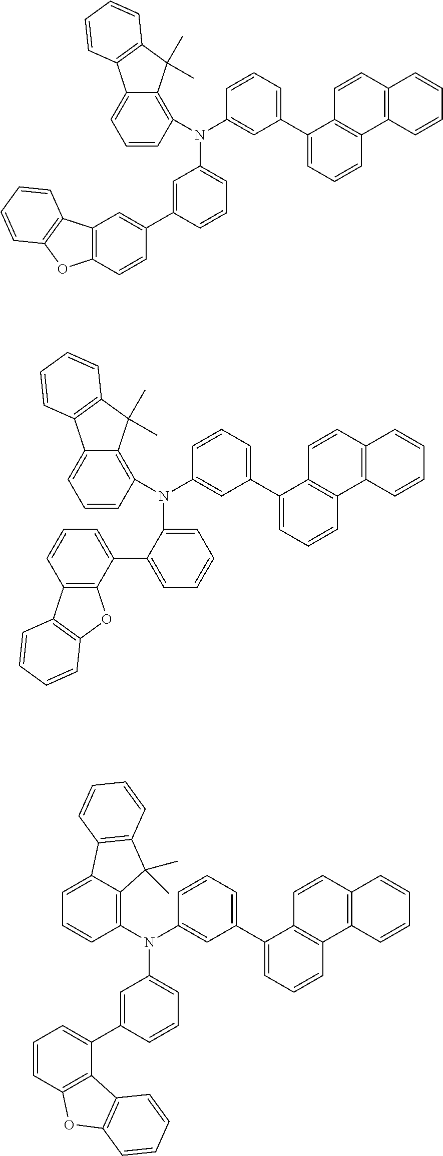 [SMILES: CC1(C)c2ccccc2-c2cccc(N(c3cccc(-c4ccc5oc6ccccc6c5c4)c3)c3cccc(-c4cccc5c4ccc4ccccc45)c3)c21 ; CC1(C)c2ccccc2-c2cccc(N(c3cccc(-c4cccc5c4ccc4ccccc45)c3)c3ccccc3-c3cccc4c3oc3ccccc34)c21 ; CC1(C)c2ccccc2-c2cccc(N(c3cccc(-c4cccc5c4ccc4ccccc45)c3)c3cccc(-c4cccc5oc6ccccc6c45)c3)c21]

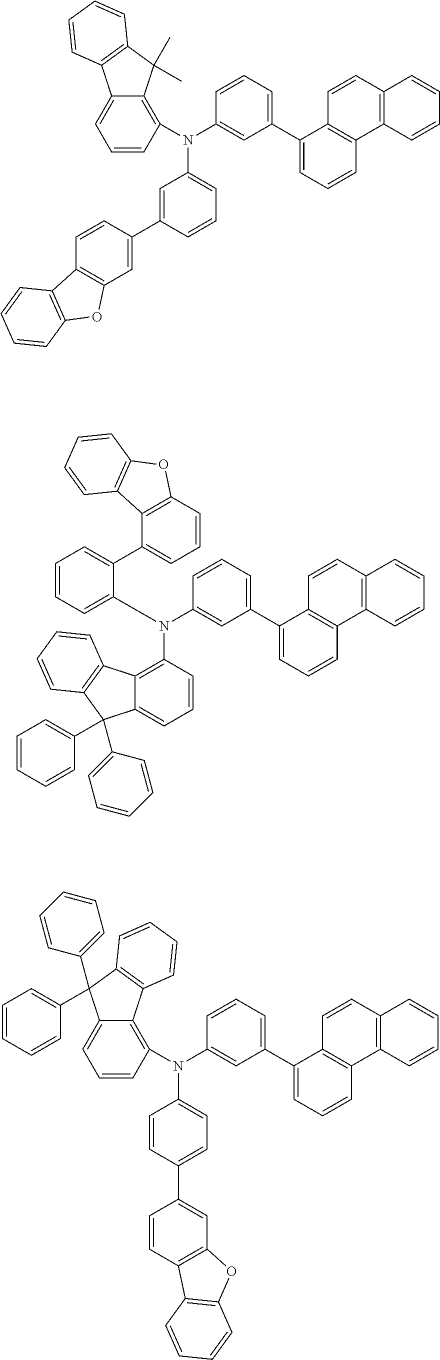 [SMILES: CC1(C)c2ccccc2-c2cccc(N(c3cccc(-c4ccc5c(c4)oc4ccccc45)c3)c3cccc(-c4cccc5c4ccc4ccccc45)c3)c21 ; c1ccc(C2(c3ccccc3)c3ccccc3-c3c(N(c4cccc(-c5cccc6c5ccc5ccccc56)c4)c4ccccc4-c4cccc5oc6ccccc6c45)cccc32)cc1 ; c1ccc(C2(c3ccccc3)c3ccccc3-c3c(N(c4ccc(-c5ccc6c(c5)oc5ccccc56)cc4)c4cccc(-c5cccc6c5ccc5ccccc56)c4)cccc32)cc1]

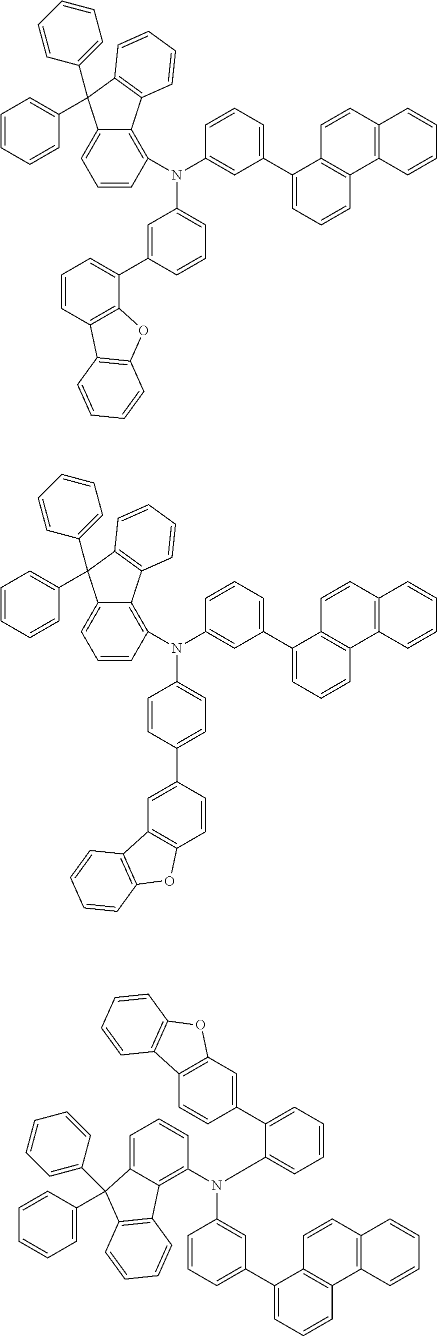 [SMILES: c1ccc(C2(c3ccccc3)c3ccccc3-c3c(N(c4cccc(-c5cccc6c5ccc5ccccc56)c4)c4cccc(-c5cccc6c5oc5ccccc56)c4)cccc32)cc1 ; c1ccc(C2(c3ccccc3)c3ccccc3-c3c(N(c4ccc(-c5ccc6oc7ccccc7c6c5)cc4)c4cccc(-c5cccc6c5ccc5ccccc56)c4)cccc32)cc1 ; c1ccc(C2(c3ccccc3)c3ccccc3-c3c(N(c4cccc(-c5cccc6c5ccc5ccccc56)c4)c4ccccc4-c4ccc5c(c4)oc4ccccc45)cccc32)cc1]

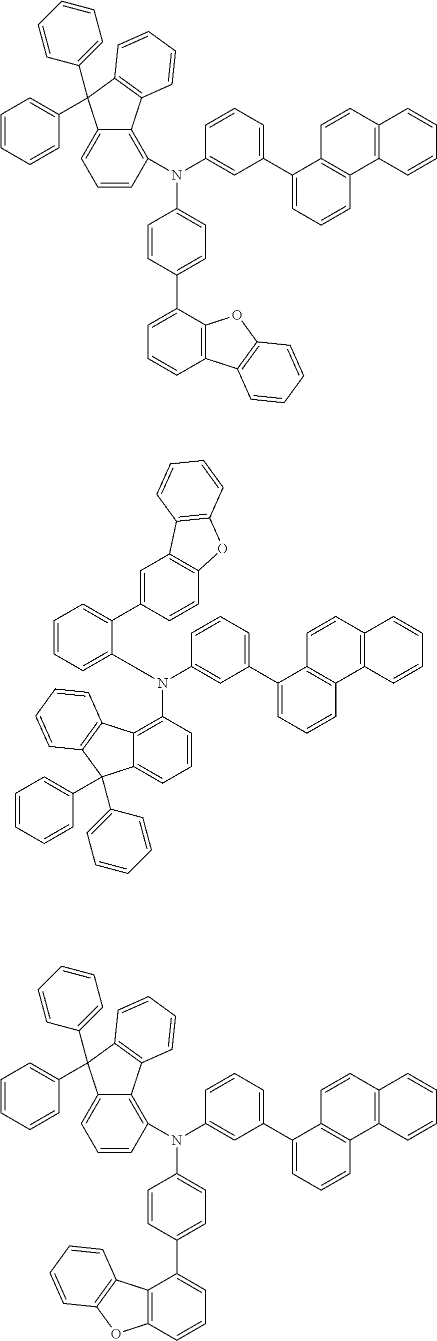 [SMILES: c1ccc(C2(c3ccccc3)c3ccccc3-c3c(N(c4ccc(-c5cccc6c5oc5ccccc56)cc4)c4cccc(-c5cccc6c5ccc5ccccc56)c4)cccc32)cc1 ; c1ccc(C2(c3ccccc3)c3ccccc3-c3c(N(c4cccc(-c5cccc6c5ccc5ccccc56)c4)c4ccccc4-c4ccc5oc6ccccc6c5c4)cccc32)cc1 ; c1ccc(C2(c3ccccc3)c3ccccc3-c3c(N(c4ccc(-c5cccc6oc7ccccc7c56)cc4)c4cccc(-c5cccc6c5ccc5ccccc56)c4)cccc32)cc1]

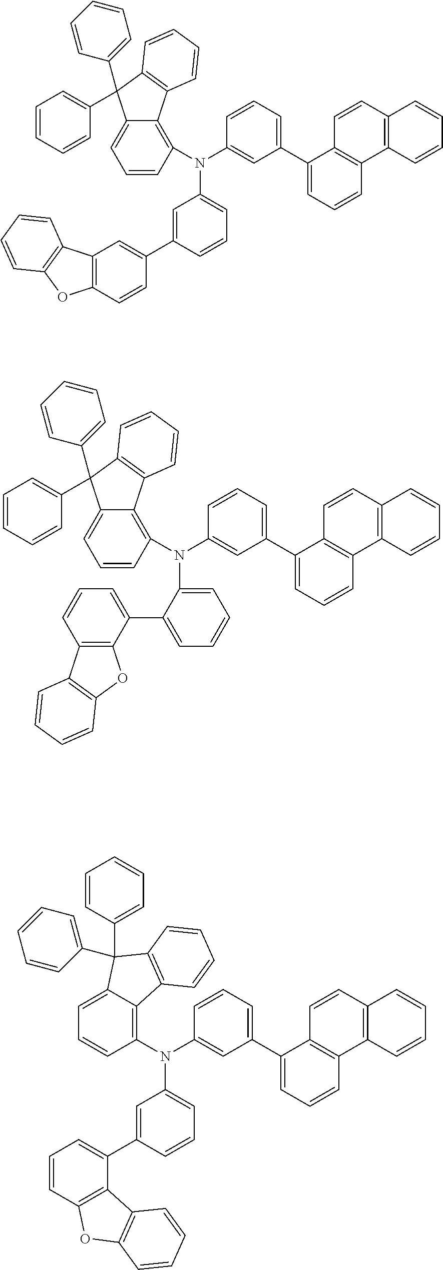 [SMILES: c1ccc(C2(c3ccccc3)c3ccccc3-c3c(N(c4cccc(-c5ccc6oc7ccccc7c6c5)c4)c4cccc(-c5cccc6c5ccc5ccccc56)c4)cccc32)cc1 ; c1ccc(C2(c3ccccc3)c3ccccc3-c3c(N(c4cccc(-c5cccc6c5ccc5ccccc56)c4)c4ccccc4-c4cccc5c4oc4ccccc45)cccc32)cc1 ; c1ccc(C2(c3ccccc3)c3ccccc3-c3c(N(c4cccc(-c5cccc6c5ccc5ccccc56)c4)c4cccc(-c5cccc6oc7ccccc7c56)c4)cccc32)cc1]

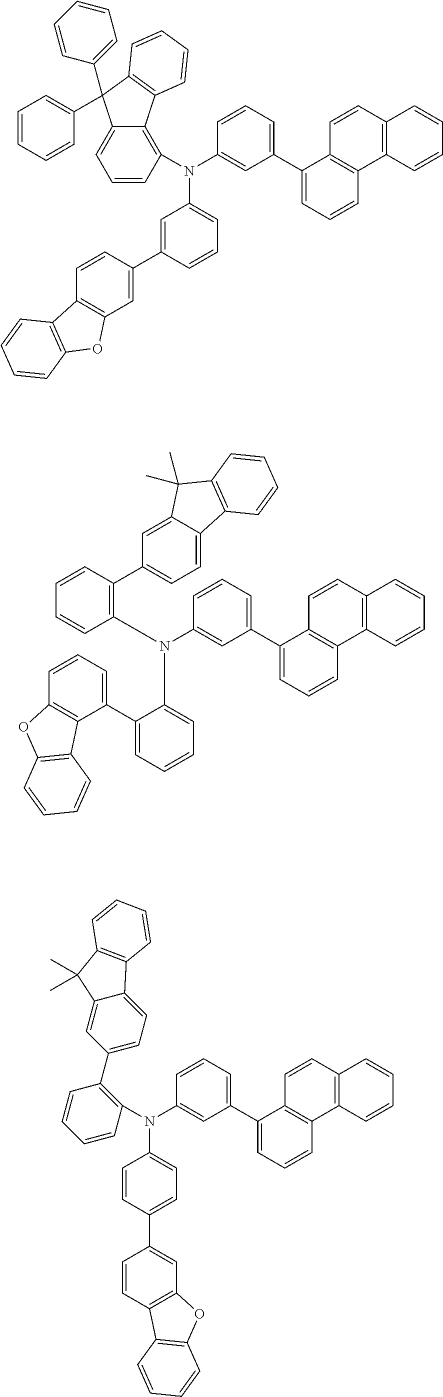 [SMILES: c1ccc(C2(c3ccccc3)c3ccccc3-c3c(N(c4cccc(-c5ccc6c(c5)oc5ccccc56)c4)c4cccc(-c5cccc6c5ccc5ccccc56)c4)cccc32)cc1 ; CC1(C)c2ccccc2-c2ccc(-c3ccccc3N(c3cccc(-c4cccc5c4ccc4ccccc45)c3)c3ccccc3-c3cccc4oc5ccccc5c34)cc21 ; CC1(C)c2ccccc2-c2ccc(-c3ccccc3N(c3ccc(-c4ccc5c(c4)oc4ccccc45)cc3)c3cccc(-c4cccc5c4ccc4ccccc45)c3)cc21]

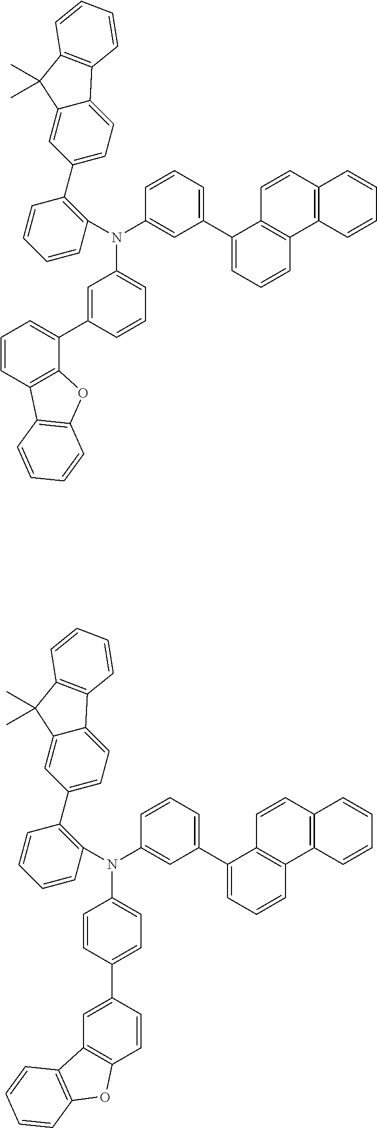 [SMILES: CC1(C)c2ccccc2-c2ccc(-c3ccccc3N(c3cccc(-c4cccc5c4ccc4ccccc45)c3)c3cccc(-c4cccc5c4oc4ccccc45)c3)cc21 ; CC1(C)c2ccccc2-c2ccc(-c3ccccc3N(c3ccc(-c4ccc5oc6ccccc6c5c4)cc3)c3cccc(-c4cccc5c4ccc4ccccc45)c3)cc21]

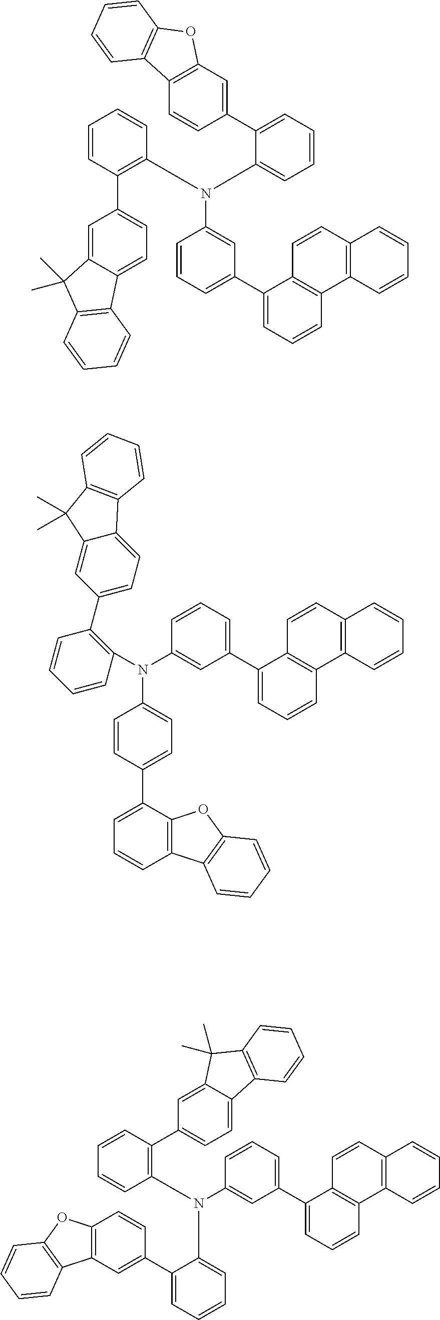 [SMILES: CC1(C)c2ccccc2-c2ccc(-c3ccccc3N(c3cccc(-c4cccc5c4ccc4ccccc45)c3)c3ccccc3-c3ccc4c(c3)oc3ccccc34)cc21 ; CC1(C)c2ccccc2-c2ccc(-c3ccccc3N(c3ccc(-c4cccc5c4oc4ccccc45)cc3)c3cccc(-c4cccc5c4ccc4ccccc45)c3)cc21 ; CC1(C)c2ccccc2-c2ccc(-c3ccccc3N(c3cccc(-c4cccc5c4ccc4ccccc45)c3)c3ccccc3-c3ccc4oc5ccccc5c4c3)cc21]

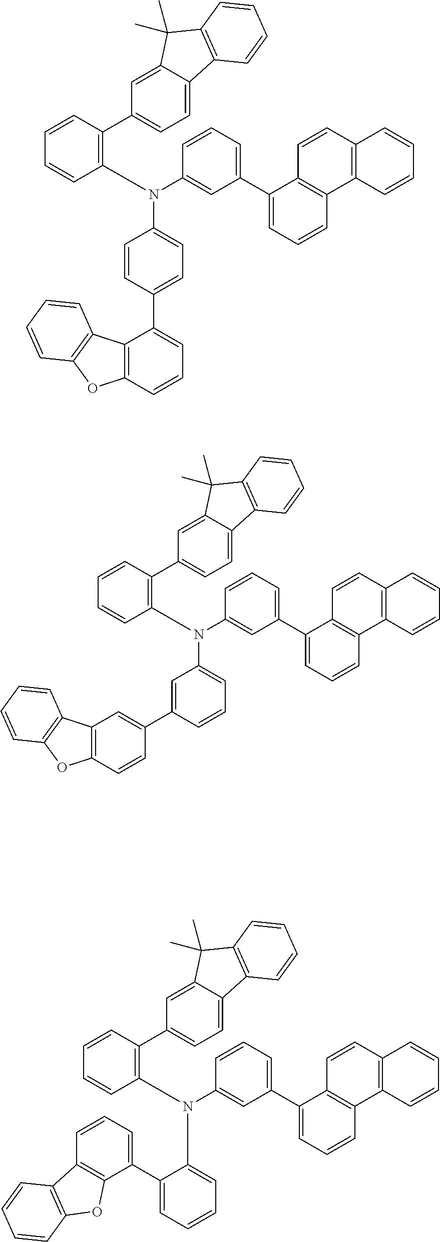 [SMILES: CC1(C)c2ccccc2-c2ccc(-c3ccccc3N(c3ccc(-c4cccc5oc6ccccc6c45)cc3)c3cccc(-c4cccc5c4ccc4ccccc45)c3)cc21 ; CC1(C)c2ccccc2-c2ccc(-c3ccccc3N(c3cccc(-c4ccc5oc6ccccc6c5c4)c3)c3cccc(-c4cccc5c4ccc4ccccc45)c3)cc21 ; CC1(C)c2ccccc2-c2ccc(-c3ccccc3N(c3cccc(-c4cccc5c4ccc4ccccc45)c3)c3ccccc3-c3cccc4c3oc3ccccc34)cc21]

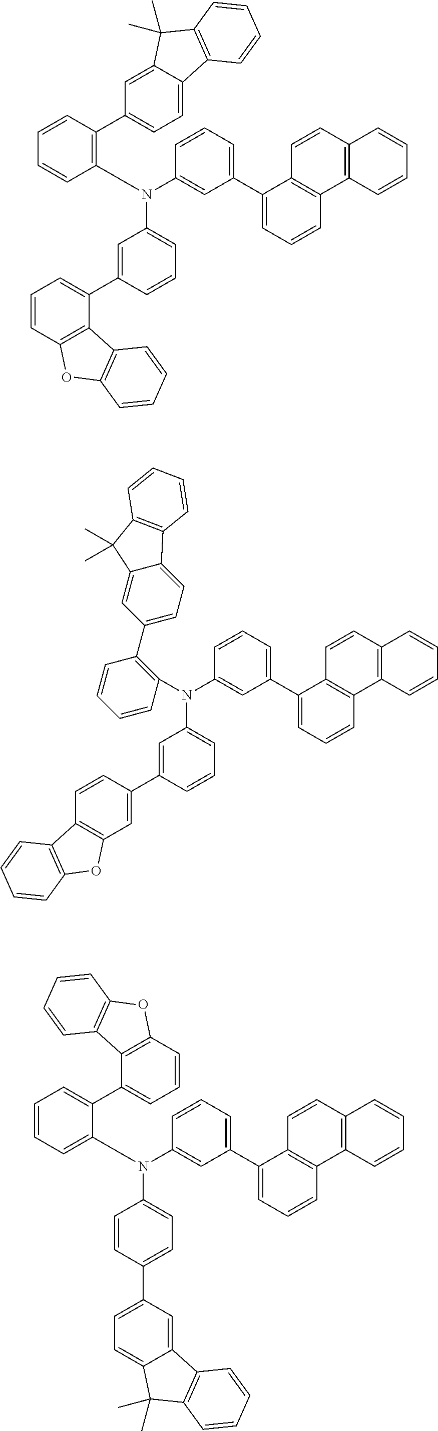 [SMILES: CC1(C)c2ccccc2-c2ccc(-c3ccccc3N(c3cccc(-c4cccc5c4ccc4ccccc45)c3)c3cccc(-c4cccc5oc6ccccc6c45)c3)cc21 ; CC1(C)c2ccccc2-c2ccc(-c3ccccc3N(c3cccc(-c4ccc5c(c4)oc4ccccc45)c3)c3cccc(-c4cccc5c4ccc4ccccc45)c3)cc21 ; CC1(C)c2ccccc2-c2cc(-c3ccc(N(c4cccc(-c5cccc6c5ccc5ccccc56)c4)c4ccccc4-c4cccc5oc6ccccc6c45)cc3)ccc21]

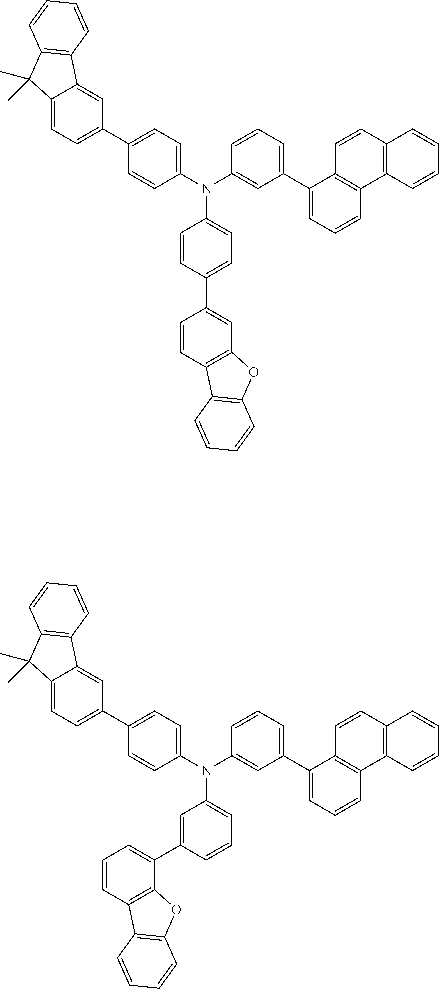 [SMILES: CC1(C)c2ccccc2-c2cc(-c3ccc(N(c4ccc(-c5ccc6c(c5)oc5ccccc56)cc4)c4cccc(-c5cccc6c5ccc5ccccc56)c4)cc3)ccc21 ; CC1(C)c2ccccc2-c2cc(-c3ccc(N(c4cccc(-c5cccc6c5ccc5ccccc56)c4)c4cccc(-c5cccc6c5oc5ccccc56)c4)cc3)ccc21]

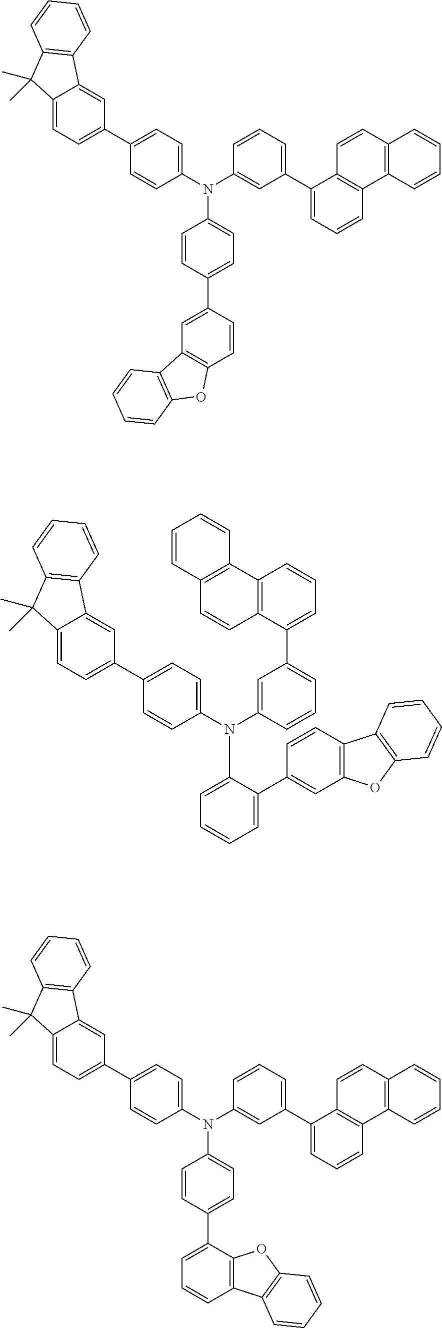 [SMILES: CC1(C)c2ccccc2-c2cc(-c3ccc(N(c4ccc(-c5ccc6oc7ccccc7c6c5)cc4)c4cccc(-c5cccc6c5ccc5ccccc56)c4)cc3)ccc21 ; CC1(C)c2ccccc2-c2cc(-c3ccc(N(c4cccc(-c5cccc6c5ccc5ccccc56)c4)c4ccccc4-c4ccc5c(c4)oc4ccccc45)cc3)ccc21 ; CC1(C)c2ccccc2-c2cc(-c3ccc(N(c4ccc(-c5cccc6c5oc5ccccc56)cc4)c4cccc(-c5cccc6c5ccc5ccccc56)c4)cc3)ccc21]

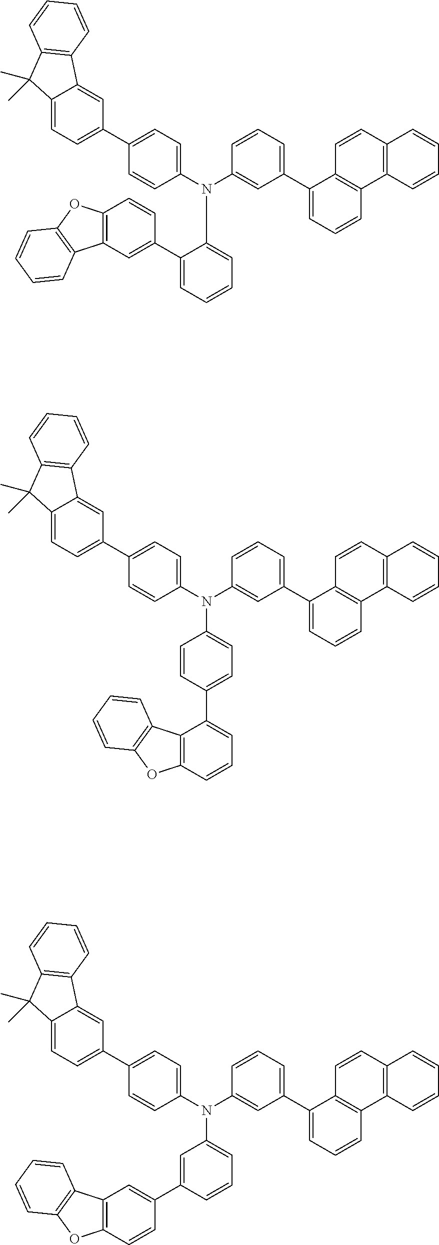 [SMILES: CC1(C)c2ccccc2-c2cc(-c3ccc(N(c4cccc(-c5cccc6c5ccc5ccccc56)c4)c4ccccc4-c4ccc5oc6ccccc6c5c4)cc3)ccc21 ; CC1(C)c2ccccc2-c2cc(-c3ccc(N(c4ccc(-c5cccc6oc7ccccc7c56)cc4)c4cccc(-c5cccc6c5ccc5ccccc56)c4)cc3)ccc21 ; CC1(C)c2ccccc2-c2cc(-c3ccc(N(c4cccc(-c5ccc6oc7ccccc7c6c5)c4)c4cccc(-c5cccc6c5ccc5ccccc56)c4)cc3)ccc21]

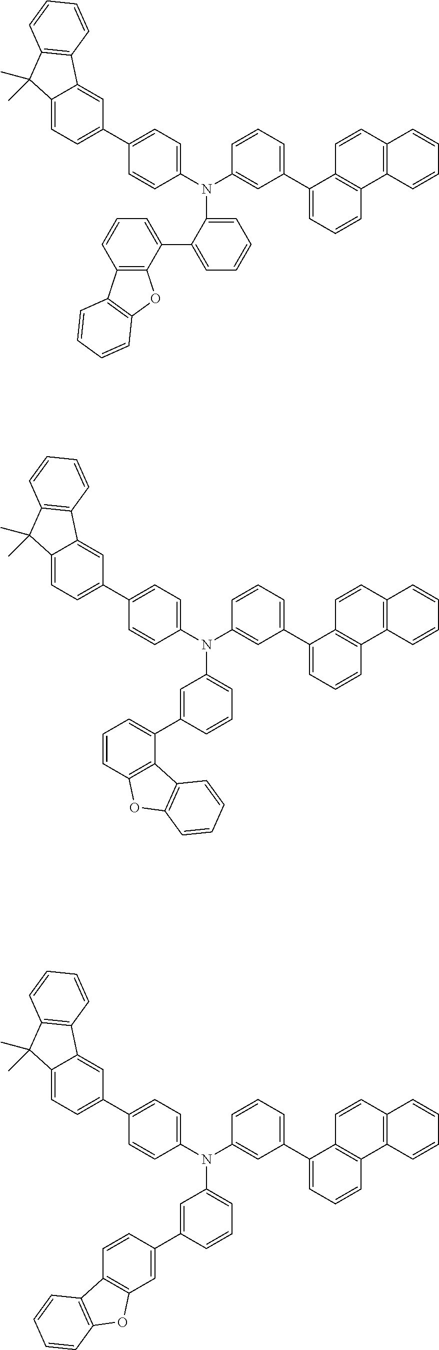 [SMILES: CC1(C)c2ccccc2-c2cc(-c3ccc(N(c4cccc(-c5cccc6c5ccc5ccccc56)c4)c4ccccc4-c4cccc5c4oc4ccccc45)cc3)ccc21 ; CC1(C)c2ccccc2-c2cc(-c3ccc(N(c4cccc(-c5cccc6c5ccc5ccccc56)c4)c4cccc(-c5cccc6oc7ccccc7c56)c4)cc3)ccc21 ; CC1(C)c2ccccc2-c2cc(-c3ccc(N(c4cccc(-c5ccc6c(c5)oc5ccccc56)c4)c4cccc(-c5cccc6c5ccc5ccccc56)c4)cc3)ccc21]

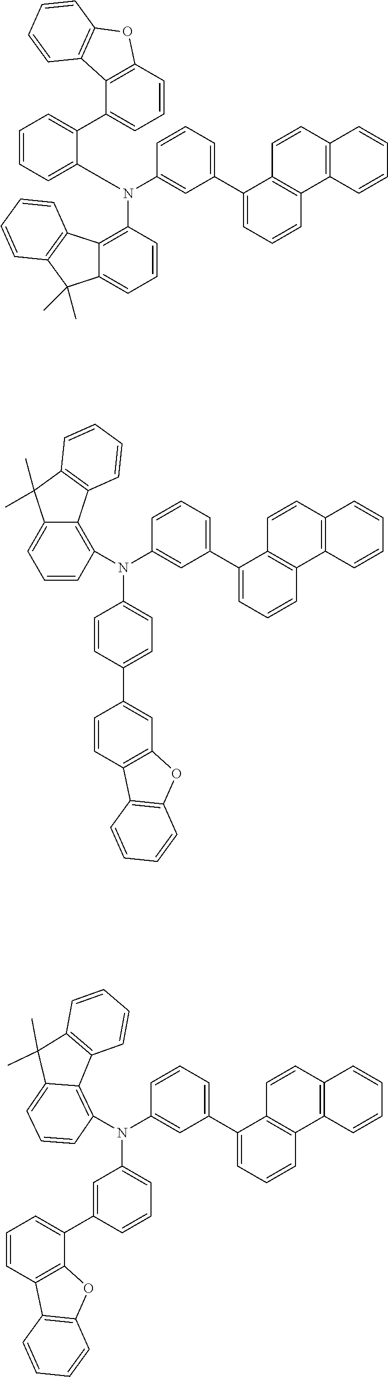 [SMILES: CC1(C)c2ccccc2-c2c(N(c3cccc(-c4cccc5c4ccc4ccccc45)c3)c3ccccc3-c3cccc4oc5ccccc5c34)cccc21 ; CC1(C)c2ccccc2-c2c(N(c3ccc(-c4ccc5c(c4)oc4ccccc45)cc3)c3cccc(-c4cccc5c4ccc4ccccc45)c3)cccc21 ; CC1(C)c2ccccc2-c2c(N(c3cccc(-c4cccc5c4ccc4ccccc45)c3)c3cccc(-c4cccc5c4oc4ccccc45)c3)cccc21]

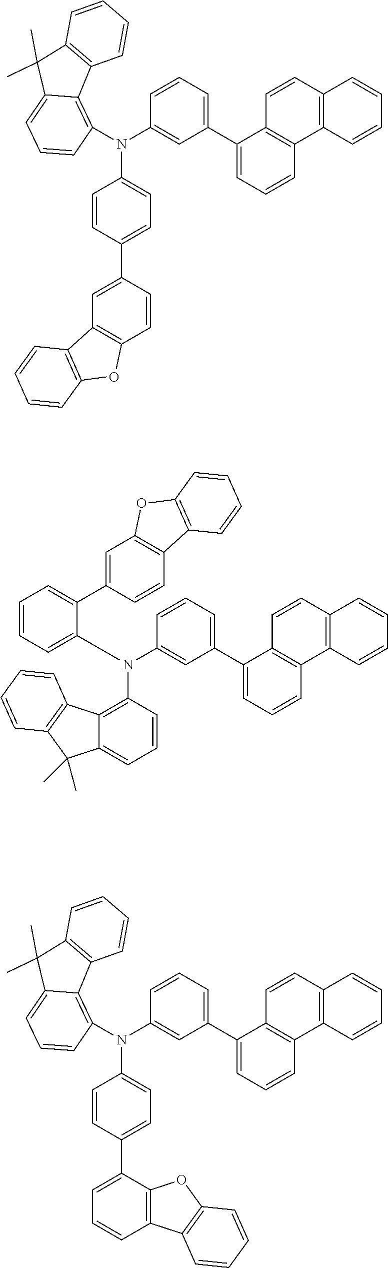 [SMILES: CC1(C)c2ccccc2-c2c(N(c3ccc(-c4ccc5oc6ccccc6c5c4)cc3)c3cccc(-c4cccc5c4ccc4ccccc45)c3)cccc21 ; CC1(C)c2ccccc2-c2c(N(c3cccc(-c4cccc5c4ccc4ccccc45)c3)c3ccccc3-c3ccc4c(c3)oc3ccccc34)cccc21 ; CC1(C)c2ccccc2-c2c(N(c3ccc(-c4cccc5c4oc4ccccc45)cc3)c3cccc(-c4cccc5c4ccc4ccccc45)c3)cccc21]

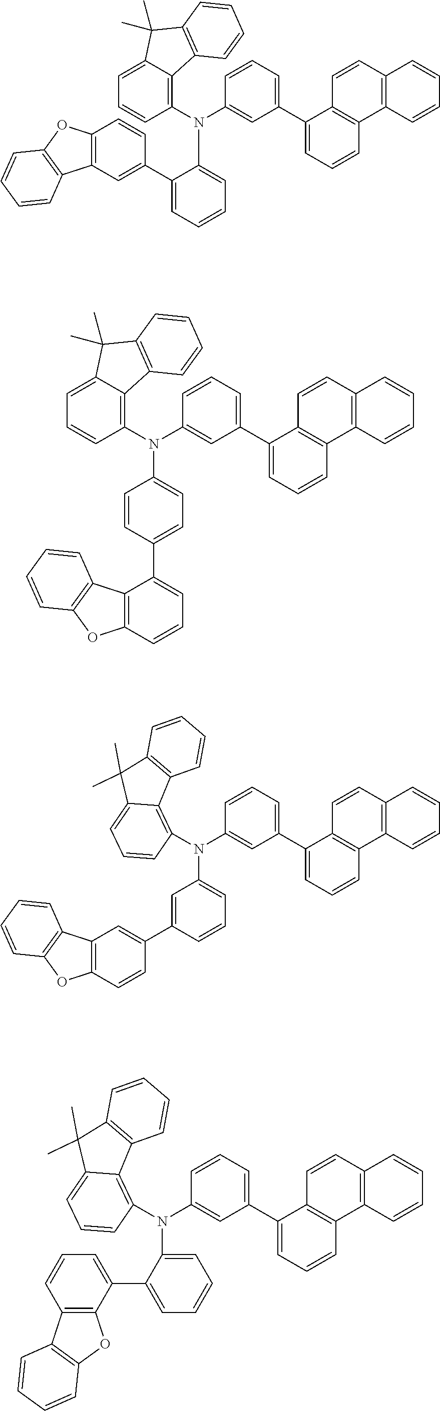 [SMILES: CC1(C)c2ccccc2-c2c(N(c3cccc(-c4cccc5c4ccc4ccccc45)c3)c3ccccc3-c3ccc4oc5ccccc5c4c3)cccc21 ; CC1(C)c2ccccc2-c2c(N(c3ccc(-c4cccc5oc6ccccc6c45)cc3)c3cccc(-c4cccc5c4ccc4ccccc45)c3)cccc21 ; CC1(C)c2ccccc2-c2c(N(c3cccc(-c4ccc5oc6ccccc6c5c4)c3)c3cccc(-c4cccc5c4ccc4ccccc45)c3)cccc21 ; CC1(C)c2ccccc2-c2c(N(c3cccc(-c4cccc5c4ccc4ccccc45)c3)c3ccccc3-c3cccc4c3oc3ccccc34)cccc21]

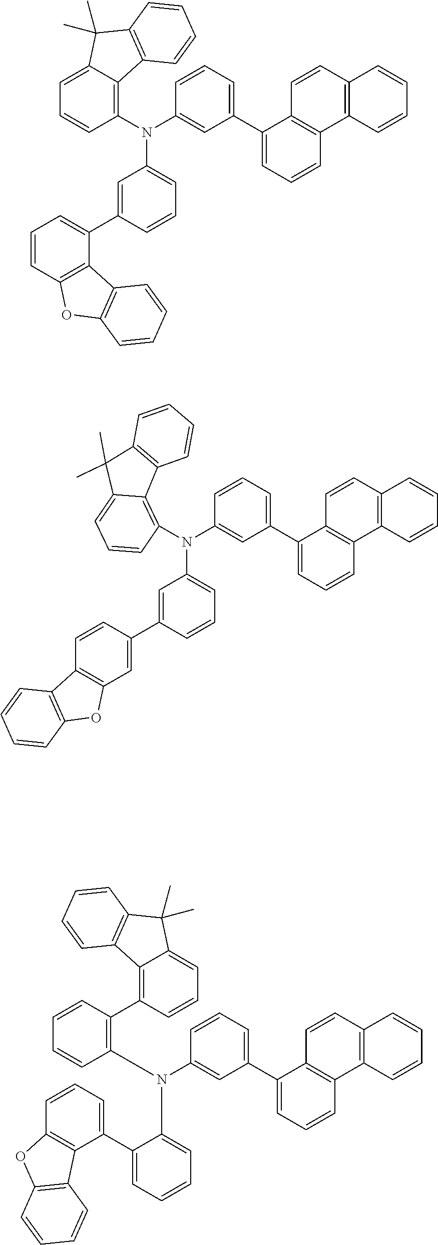 [SMILES: CC1(C)c2ccccc2-c2c(N(c3cccc(-c4cccc5c4ccc4ccccc45)c3)c3cccc(-c4cccc5oc6ccccc6c45)c3)cccc21 ; CC1(C)c2ccccc2-c2c(N(c3cccc(-c4ccc5c(c4)oc4ccccc45)c3)c3cccc(-c4cccc5c4ccc4ccccc45)c3)cccc21 ; CC1(C)c2ccccc2-c2c(-c3ccccc3N(c3cccc(-c4cccc5c4ccc4ccccc45)c3)c3ccccc3-c3cccc4oc5ccccc5c34)cccc21]

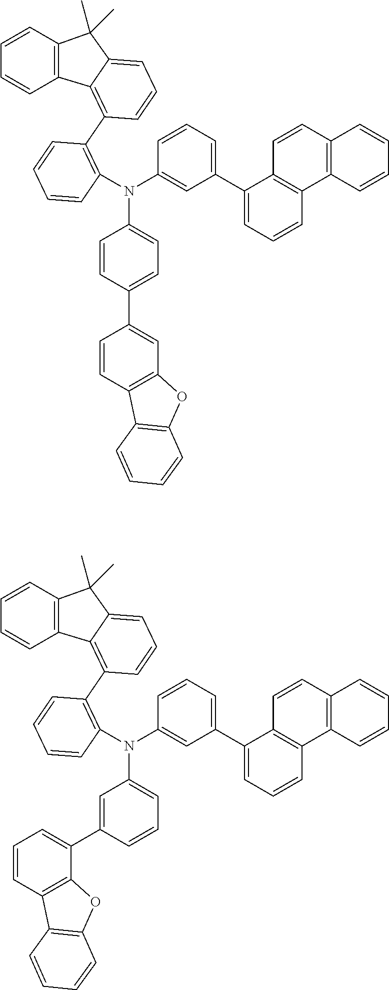 [SMILES: CC1(C)c2ccccc2-c2c(-c3ccccc3N(c3ccc(-c4ccc5c(c4)oc4ccccc45)cc3)c3cccc(-c4cccc5c4ccc4ccccc45)c3)cccc21 ; CC1(C)c2ccccc2-c2c(-c3ccccc3N(c3cccc(-c4cccc5c4ccc4ccccc45)c3)c3cccc(-c4cccc5c4oc4ccccc45)c3)cccc21]

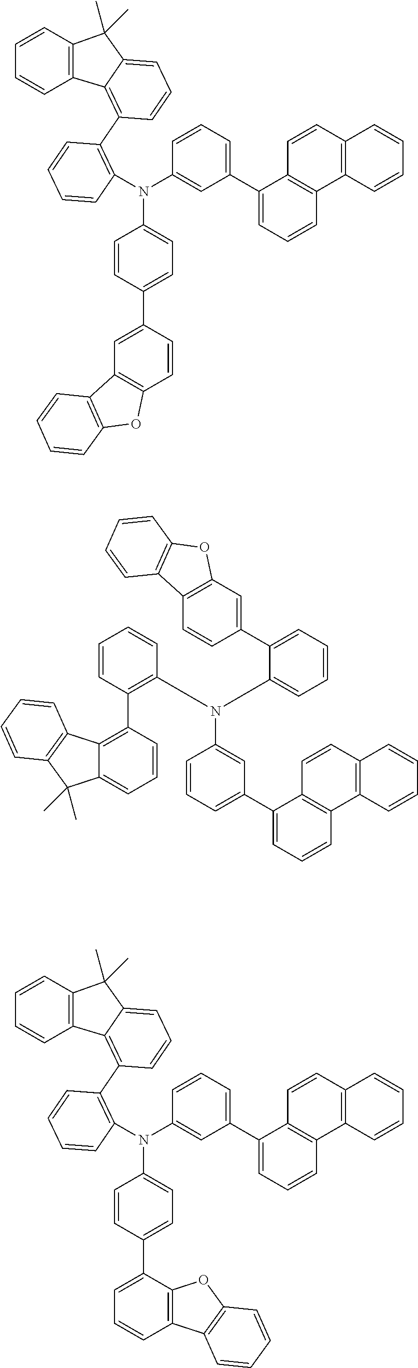 [SMILES: CC1(C)c2ccccc2-c2c(-c3ccccc3N(c3ccc(-c4ccc5oc6ccccc6c5c4)cc3)c3cccc(-c4cccc5c4ccc4ccccc45)c3)cccc21 ; CC1(C)c2ccccc2-c2c(-c3ccccc3N(c3cccc(-c4cccc5c4ccc4ccccc45)c3)c3ccccc3-c3ccc4c(c3)oc3ccccc34)cccc21 ; CC1(C)c2ccccc2-c2c(-c3ccccc3N(c3ccc(-c4cccc5c4oc4ccccc45)cc3)c3cccc(-c4cccc5c4ccc4ccccc45)c3)cccc21]

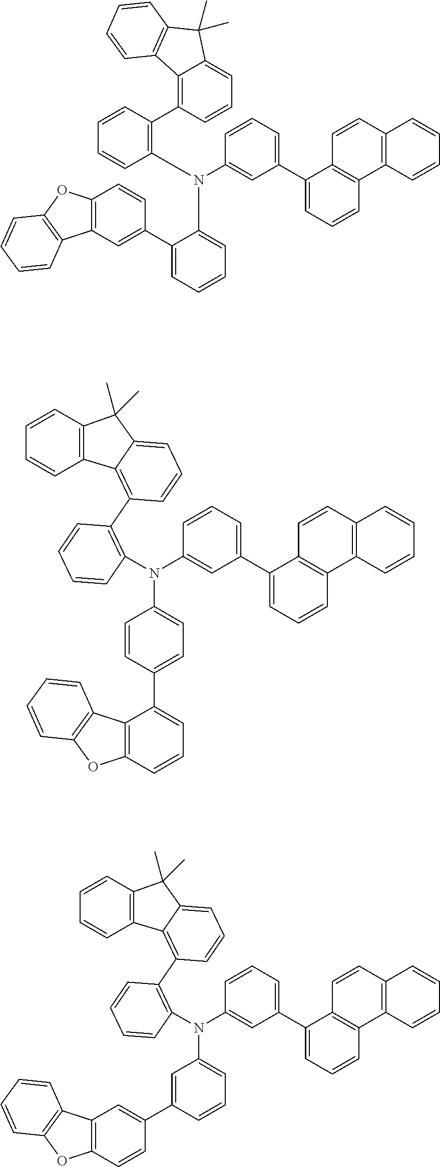 [SMILES: CC1(C)c2ccccc2-c2c(-c3ccccc3N(c3cccc(-c4cccc5c4ccc4ccccc45)c3)c3ccccc3-c3ccc4oc5ccccc5c4c3)cccc21 ; CC1(C)c2ccccc2-c2c(-c3ccccc3N(c3ccc(-c4cccc5oc6ccccc6c45)cc3)c3cccc(-c4cccc5c4ccc4ccccc45)c3)cccc21 ; CC1(C)c2ccccc2-c2c(-c3ccccc3N(c3cccc(-c4ccc5oc6ccccc6c5c4)c3)c3cccc(-c4cccc5c4ccc4ccccc45)c3)cccc21]

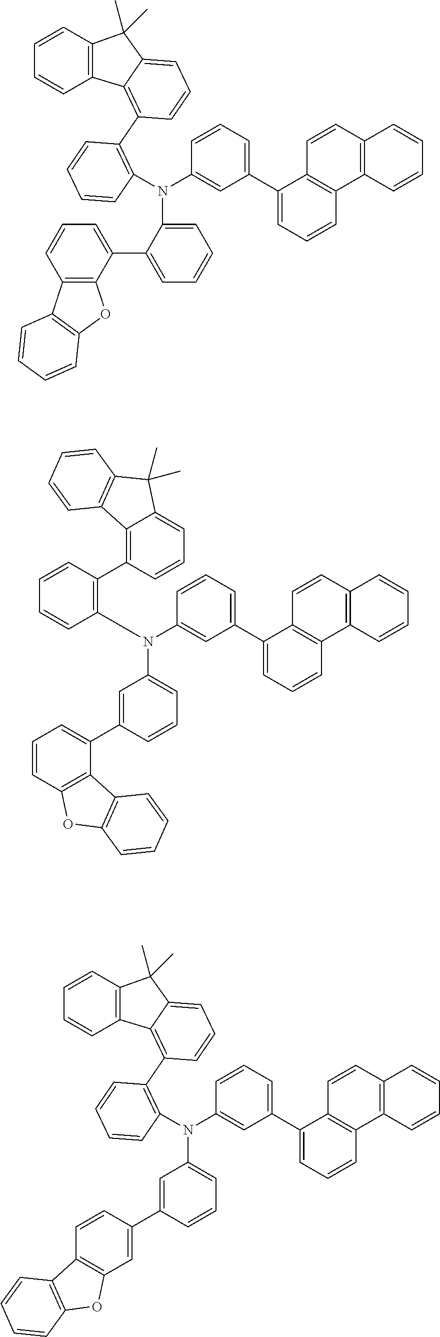 [SMILES: CC1(C)c2ccccc2-c2c(-c3ccccc3N(c3cccc(-c4cccc5c4ccc4ccccc45)c3)c3ccccc3-c3cccc4c3oc3ccccc34)cccc21 ; CC1(C)c2ccccc2-c2c(-c3ccccc3N(c3cccc(-c4cccc5c4ccc4ccccc45)c3)c3cccc(-c4cccc5oc6ccccc6c45)c3)cccc21 ; CC1(C)c2ccccc2-c2c(-c3ccccc3N(c3cccc(-c4ccc5c(c4)oc4ccccc45)c3)c3cccc(-c4cccc5c4ccc4ccccc45)c3)cccc21]

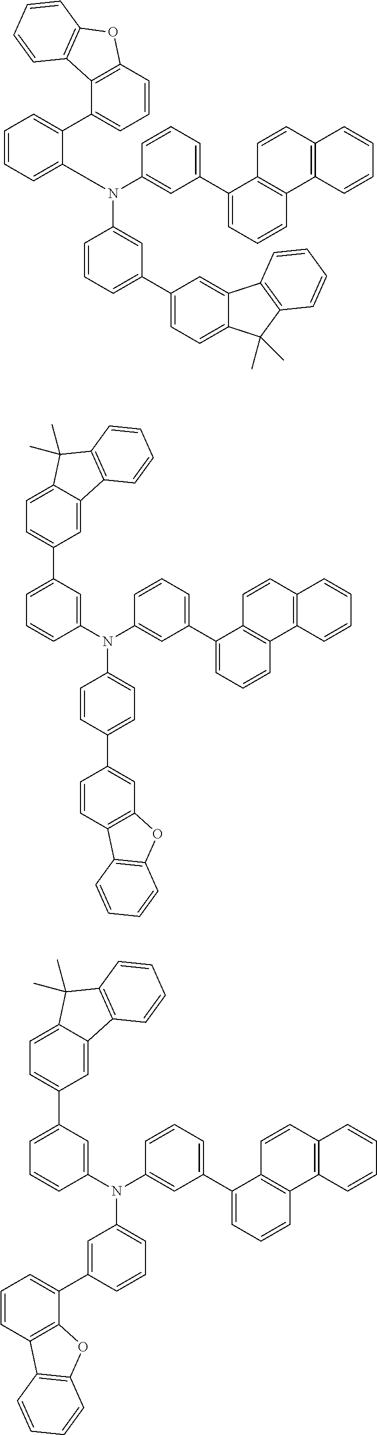 [SMILES: CC1(C)c2ccccc2-c2cc(-c3cccc(N(c4cccc(-c5cccc6c5ccc5ccccc56)c4)c4ccccc4-c4cccc5oc6ccccc6c45)c3)ccc21 ; CC1(C)c2ccccc2-c2cc(-c3cccc(N(c4ccc(-c5ccc6c(c5)oc5ccccc56)cc4)c4cccc(-c5cccc6c5ccc5ccccc56)c4)c3)ccc21 ; CC1(C)c2ccccc2-c2cc(-c3cccc(N(c4cccc(-c5cccc6c5ccc5ccccc56)c4)c4cccc(-c5cccc6c5oc5ccccc56)c4)c3)ccc21]

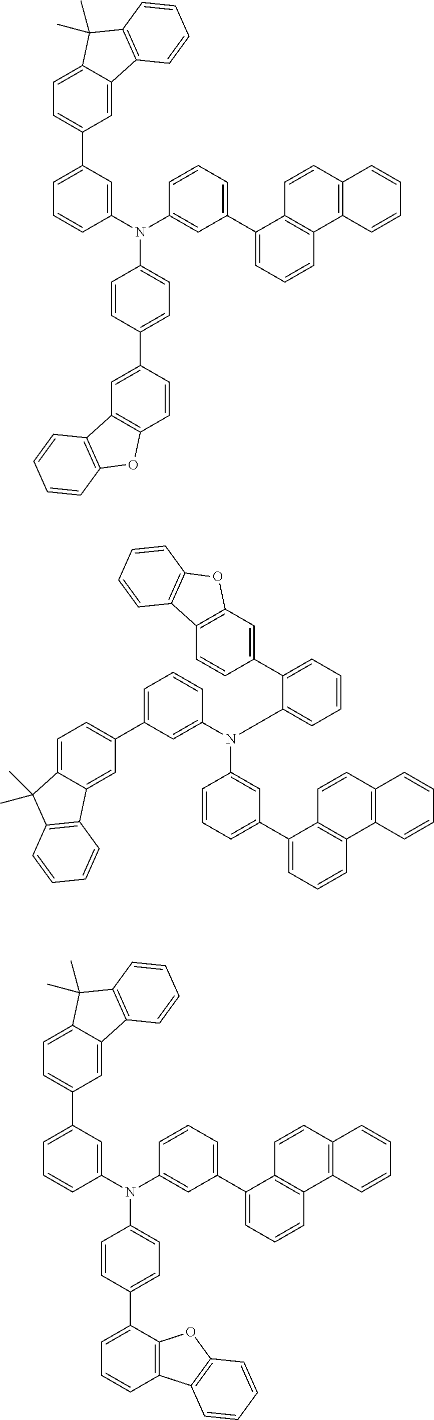 [SMILES: CC1(C)c2ccccc2-c2cc(-c3cccc(N(c4ccc(-c5ccc6oc7ccccc7c6c5)cc4)c4cccc(-c5cccc6c5ccc5ccccc56)c4)c3)ccc21 ; CC1(C)c2ccccc2-c2cc(-c3cccc(N(c4cccc(-c5cccc6c5ccc5ccccc56)c4)c4ccccc4-c4ccc5c(c4)oc4ccccc45)c3)ccc21 ; CC1(C)c2ccccc2-c2cc(-c3cccc(N(c4ccc(-c5cccc6c5oc5ccccc56)cc4)c4cccc(-c5cccc6c5ccc5ccccc56)c4)c3)ccc21]

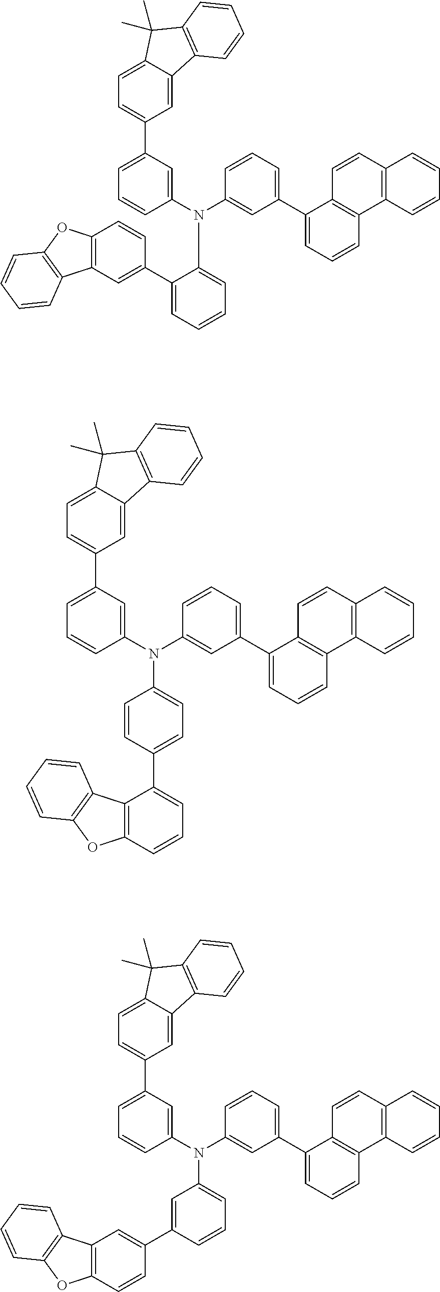 [SMILES: CC1(C)c2ccccc2-c2cc(-c3cccc(N(c4cccc(-c5cccc6c5ccc5ccccc56)c4)c4ccccc4-c4ccc5oc6ccccc6c5c4)c3)ccc21 ; CC1(C)c2ccccc2-c2cc(-c3cccc(N(c4ccc(-c5cccc6oc7ccccc7c56)cc4)c4cccc(-c5cccc6c5ccc5ccccc56)c4)c3)ccc21 ; CC1(C)c2ccccc2-c2cc(-c3cccc(N(c4cccc(-c5ccc6oc7ccccc7c6c5)c4)c4cccc(-c5cccc6c5ccc5ccccc56)c4)c3)ccc21]

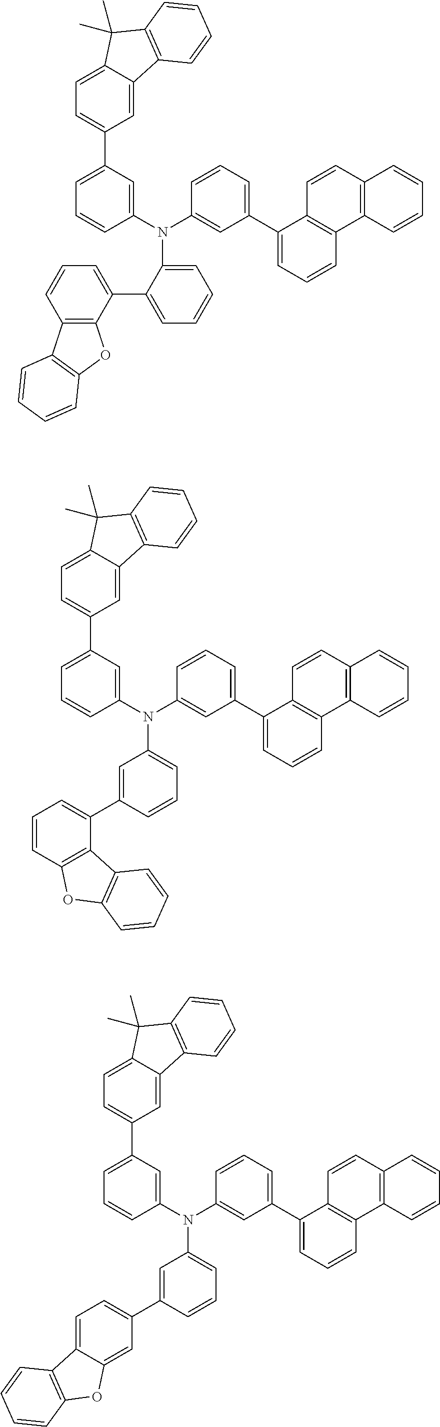 [SMILES: CC1(C)c2ccccc2-c2cc(-c3cccc(N(c4cccc(-c5cccc6c5ccc5ccccc56)c4)c4ccccc4-c4cccc5c4oc4ccccc45)c3)ccc21 ; CC1(C)c2ccccc2-c2cc(-c3cccc(N(c4cccc(-c5cccc6c5ccc5ccccc56)c4)c4cccc(-c5cccc6oc7ccccc7c56)c4)c3)ccc21 ; CC1(C)c2ccccc2-c2cc(-c3cccc(N(c4cccc(-c5ccc6c(c5)oc5ccccc56)c4)c4cccc(-c5cccc6c5ccc5ccccc56)c4)c3)ccc21]

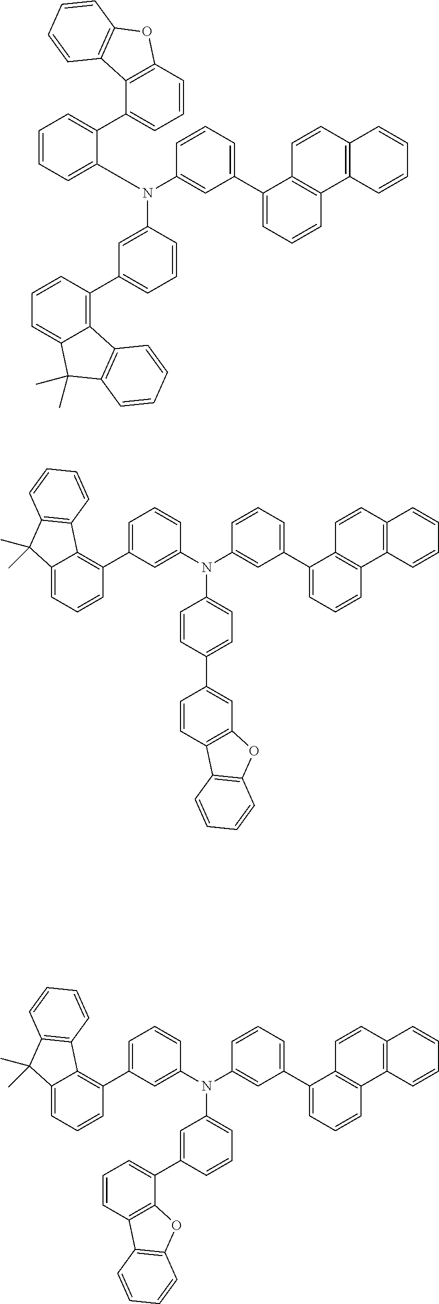 [SMILES: CC1(C)c2ccccc2-c2c(-c3cccc(N(c4cccc(-c5cccc6c5ccc5ccccc56)c4)c4ccccc4-c4cccc5oc6ccccc6c45)c3)cccc21 ; CC1(C)c2ccccc2-c2c(-c3cccc(N(c4ccc(-c5ccc6c(c5)oc5ccccc56)cc4)c4cccc(-c5cccc6c5ccc5ccccc56)c4)c3)cccc21 ; CC1(C)c2ccccc2-c2c(-c3cccc(N(c4cccc(-c5cccc6c5ccc5ccccc56)c4)c4cccc(-c5cccc6c5oc5ccccc56)c4)c3)cccc21]

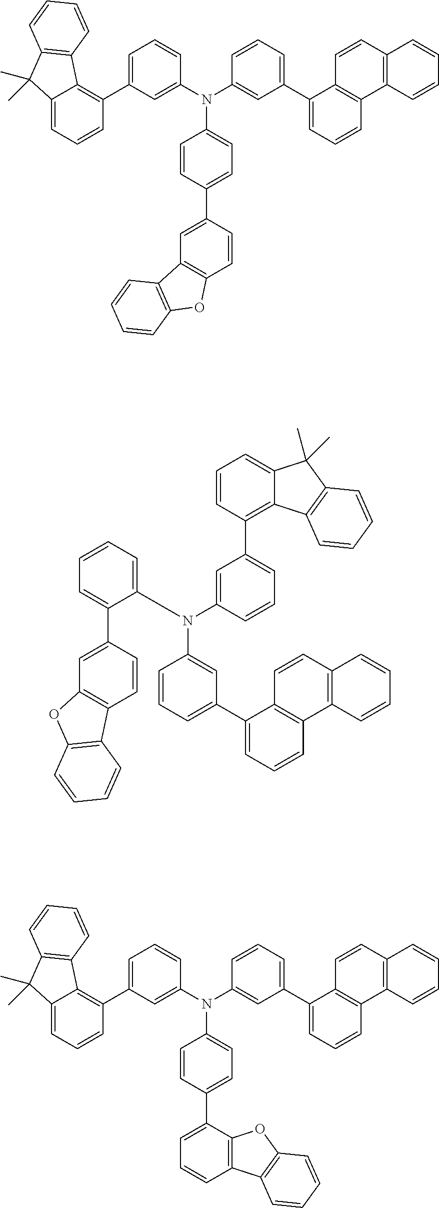 [SMILES: CC1(C)c2ccccc2-c2c(-c3cccc(N(c4ccc(-c5ccc6oc7ccccc7c6c5)cc4)c4cccc(-c5cccc6c5ccc5ccccc56)c4)c3)cccc21 ; CC1(C)c2ccccc2-c2c(-c3cccc(N(c4cccc(-c5cccc6c5ccc5ccccc56)c4)c4ccccc4-c4ccc5c(c4)oc4ccccc45)c3)cccc21 ; CC1(C)c2ccccc2-c2c(-c3cccc(N(c4ccc(-c5cccc6c5oc5ccccc56)cc4)c4cccc(-c5cccc6c5ccc5ccccc56)c4)c3)cccc21]

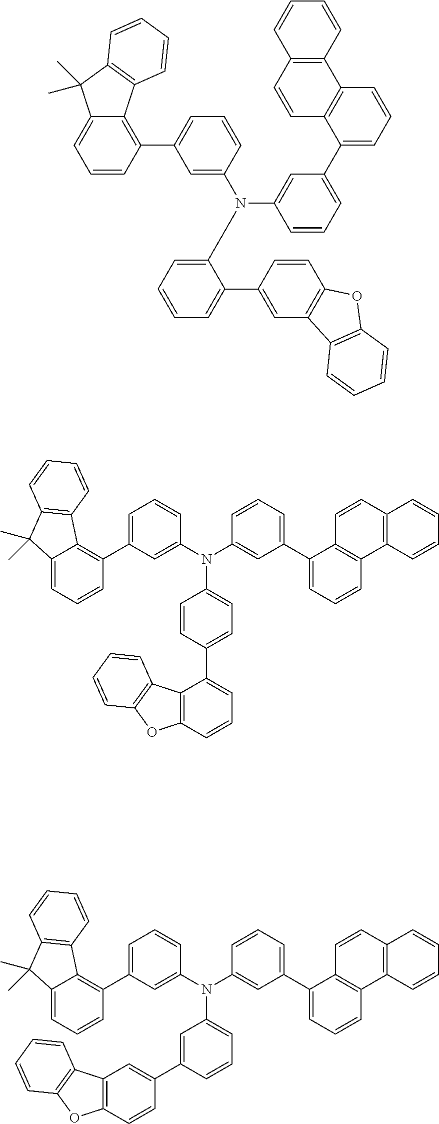 [SMILES: CC1(C)c2ccccc2-c2c(-c3cccc(N(c4cccc(-c5cccc6c5ccc5ccccc56)c4)c4ccccc4-c4ccc5oc6ccccc6c5c4)c3)cccc21 ; CC1(C)c2ccccc2-c2c(-c3cccc(N(c4ccc(-c5cccc6oc7ccccc7c56)cc4)c4cccc(-c5cccc6c5ccc5ccccc56)c4)c3)cccc21 ; CC1(C)c2ccccc2-c2c(-c3cccc(N(c4cccc(-c5ccc6oc7ccccc7c6c5)c4)c4cccc(-c5cccc6c5ccc5ccccc56)c4)c3)cccc21]

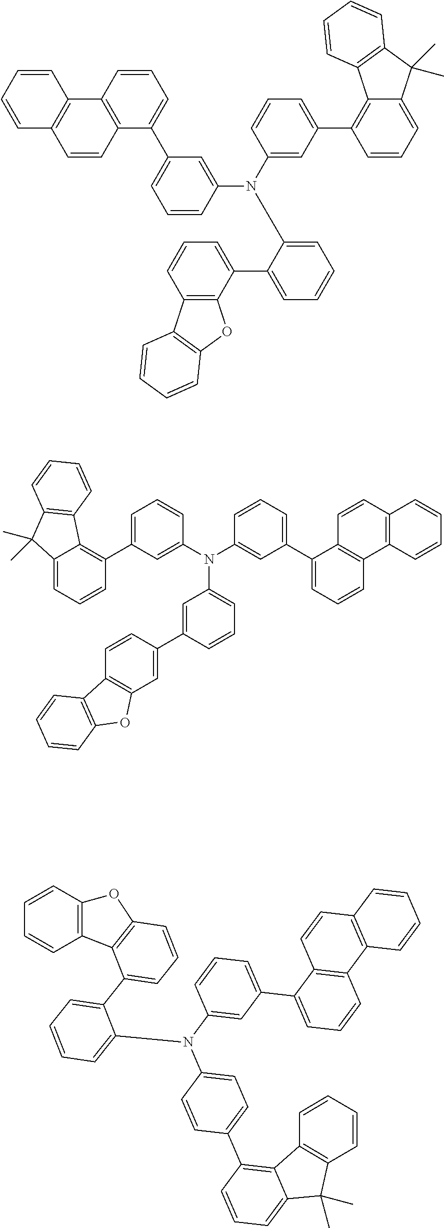

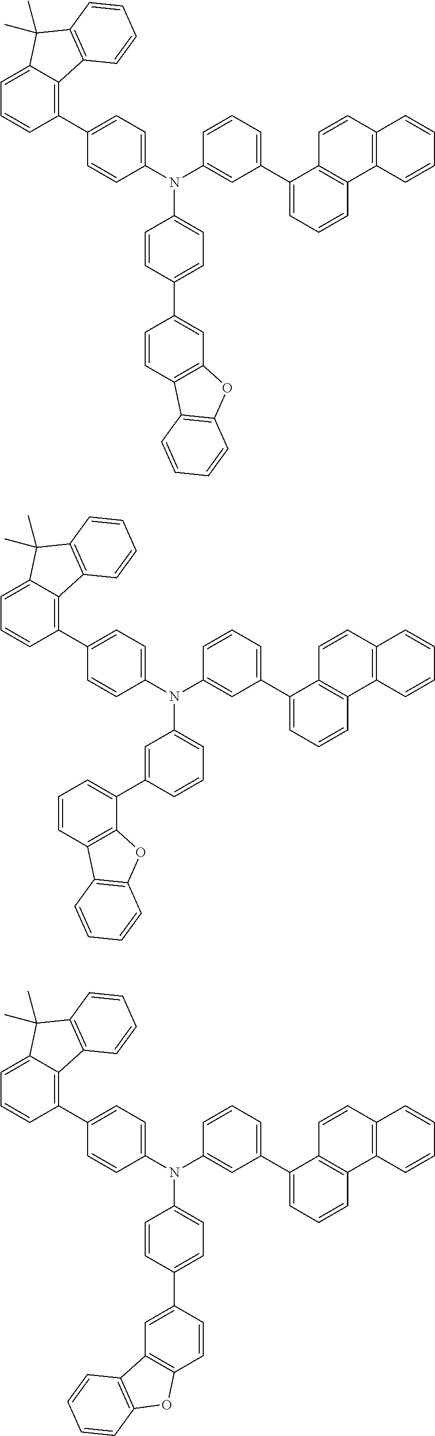 [SMILES: CC1(C)c2ccccc2-c2c(-c3ccc(N(c4ccc(-c5ccc6c(c5)oc5ccccc56)cc4)c4cccc(-c5cccc6c5ccc5ccccc56)c4)cc3)cccc21 ; CC1(C)c2ccccc2-c2c(-c3ccc(N(c4cccc(-c5cccc6c5ccc5ccccc56)c4)c4cccc(-c5cccc6c5oc5ccccc56)c4)cc3)cccc21 ; CC1(C)c2ccccc2-c2c(-c3ccc(N(c4ccc(-c5ccc6oc7ccccc7c6c5)cc4)c4cccc(-c5cccc6c5ccc5ccccc56)c4)cc3)cccc21]

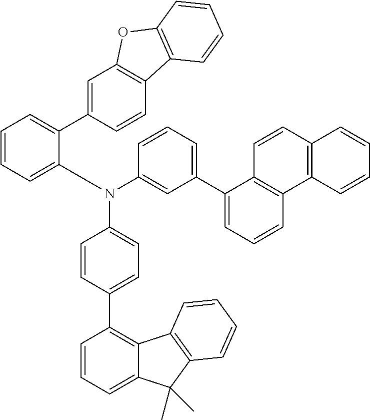 [SMILES: CC1(C)c2ccccc2-c2c(-c3ccc(N(c4cccc(-c5cccc6c5ccc5ccccc56)c4)c4ccccc4-c4ccc5c(c4)oc4ccccc45)cc3)cccc21]

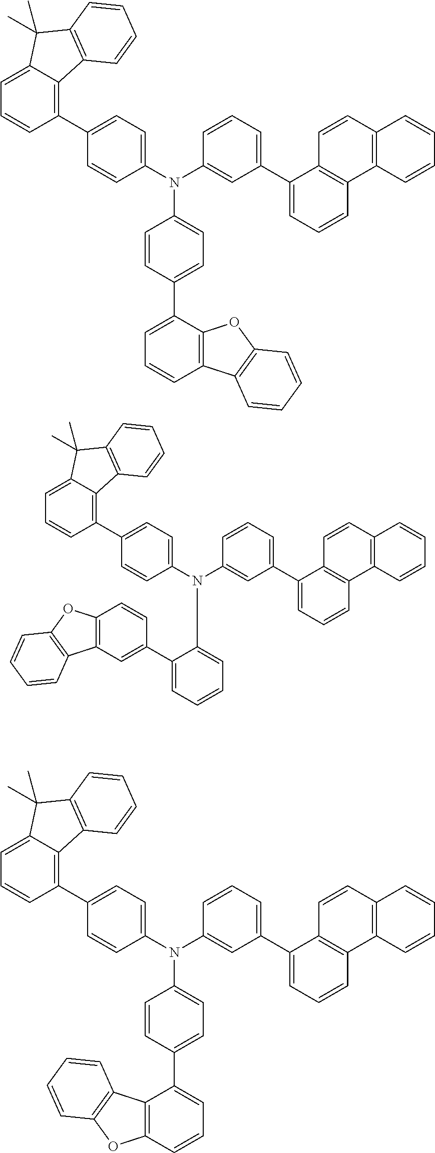 [SMILES: CC1(C)c2ccccc2-c2c(-c3ccc(N(c4ccc(-c5cccc6c5oc5ccccc56)cc4)c4cccc(-c5cccc6c5ccc5ccccc56)c4)cc3)cccc21 ; CC1(C)c2ccccc2-c2c(-c3ccc(N(c4cccc(-c5cccc6c5ccc5ccccc56)c4)c4ccccc4-c4ccc5oc6ccccc6c5c4)cc3)cccc21 ; CC1(C)c2ccccc2-c2c(-c3ccc(N(c4ccc(-c5cccc6oc7ccccc7c56)cc4)c4cccc(-c5cccc6c5ccc5ccccc56)c4)cc3)cccc21]

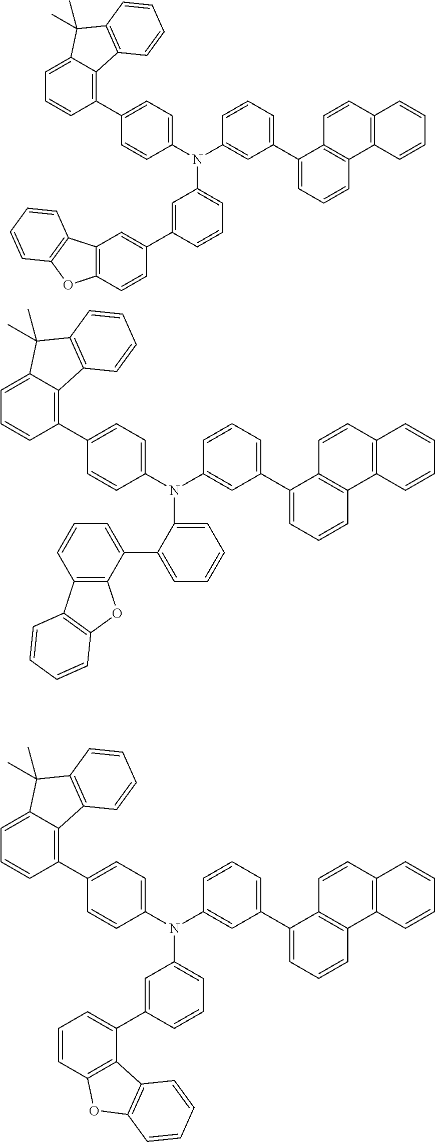

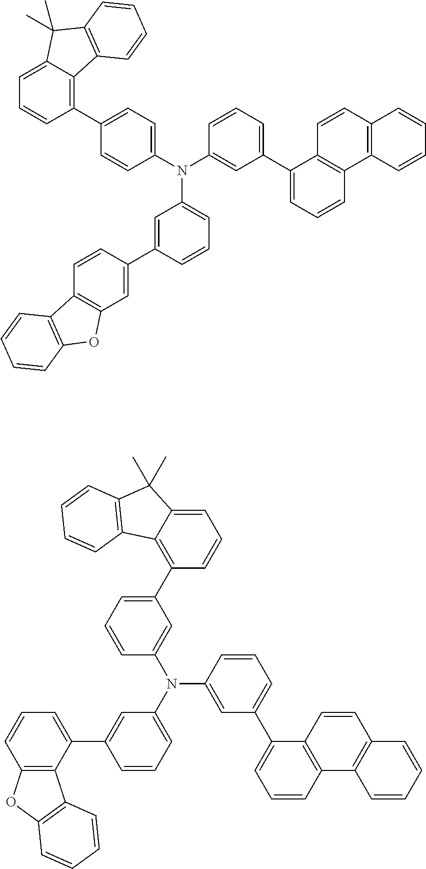 [SMILES: CC1(C)c2ccccc2-c2c(-c3ccc(N(c4cccc(-c5ccc6c(c5)oc5ccccc56)c4)c4cccc(-c5cccc6c5ccc5ccccc56)c4)cc3)cccc21 ; CC1(C)c2ccccc2-c2c(-c3cccc(N(c4cccc(-c5cccc6c5ccc5ccccc56)c4)c4cccc(-c5cccc6oc7ccccc7c56)c4)c3)cccc21]

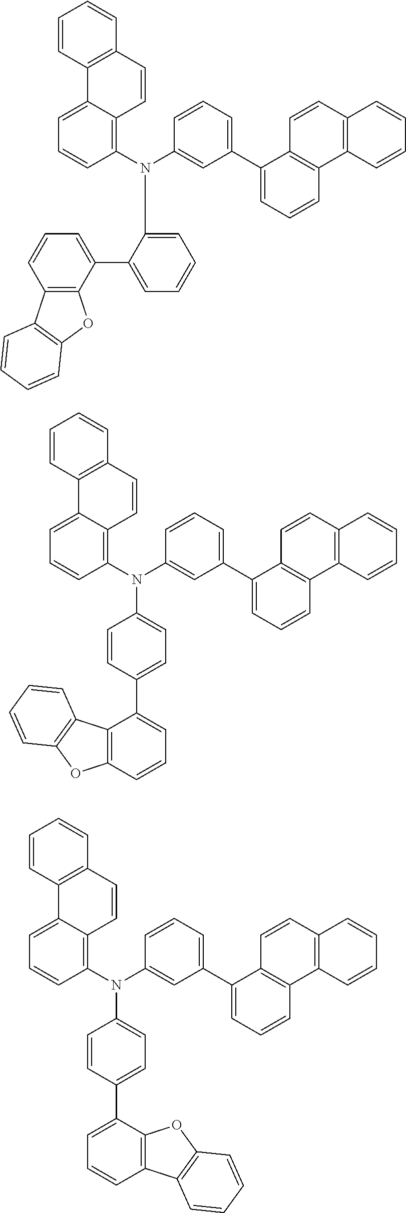

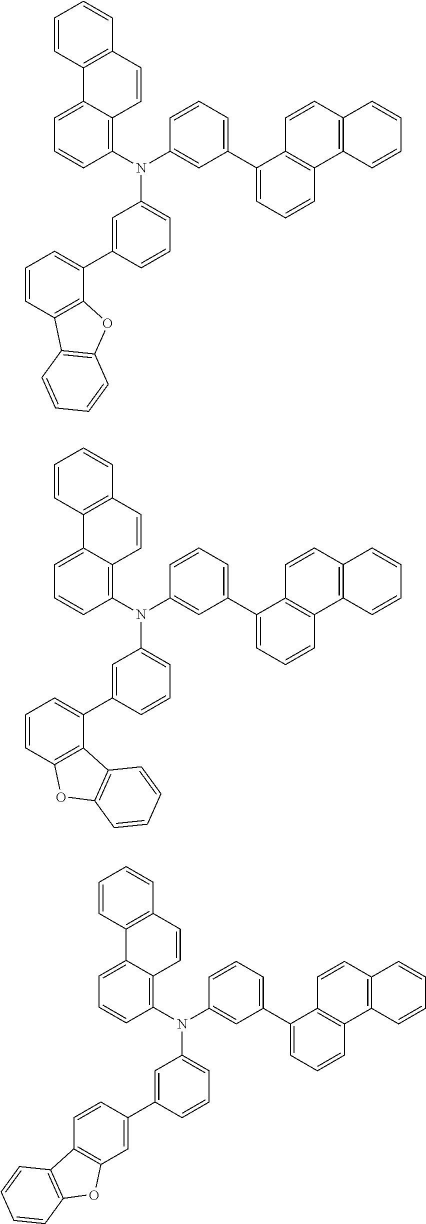

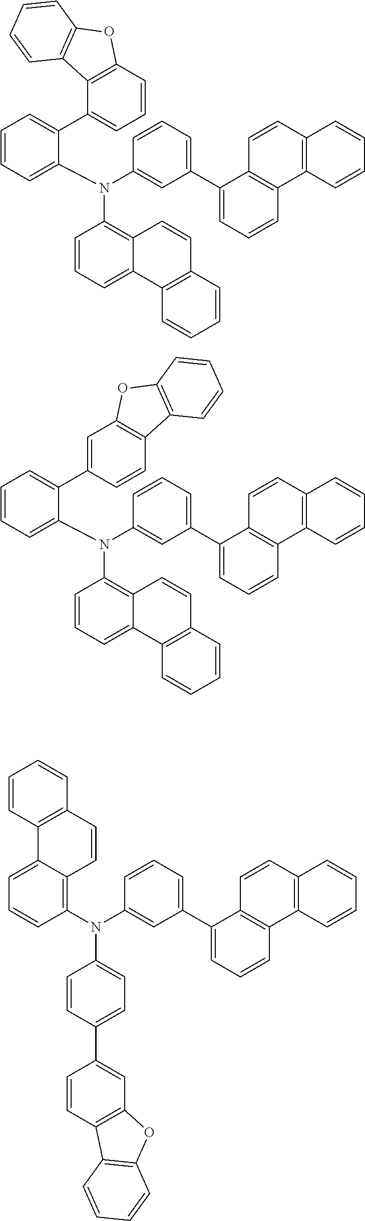

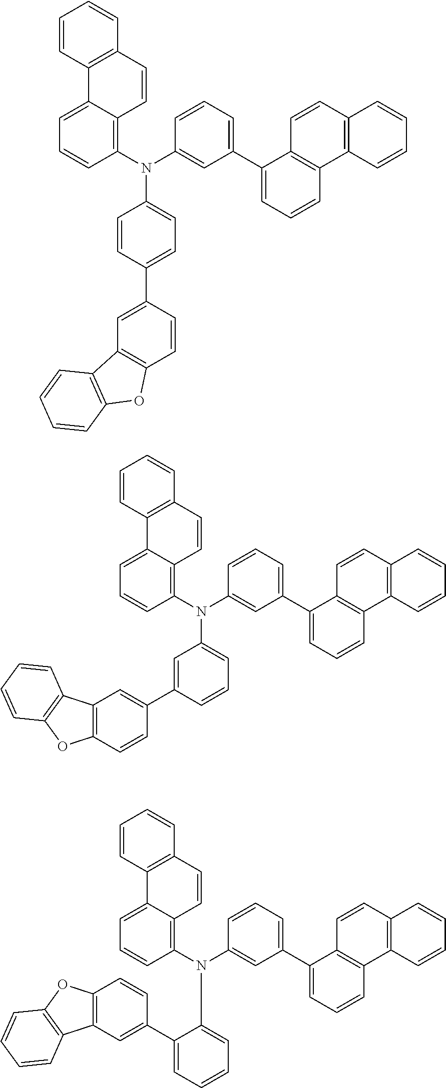 [SMILES: c1cc(-c2cccc3c2ccc2ccccc23)cc(N(c2ccc(-c3ccc4oc5ccccc5c4c3)cc2)c2cccc3c2ccc2ccccc23)c1 ; c1cc(-c2ccc3oc4ccccc4c3c2)cc(N(c2cccc(-c3cccc4c3ccc3ccccc34)c2)c2cccc3c2ccc2ccccc23)c1 ; c1cc(-c2cccc3c2ccc2ccccc23)cc(N(c2ccccc2-c2ccc3oc4ccccc4c3c2)c2cccc3c2ccc2ccccc23)c1]

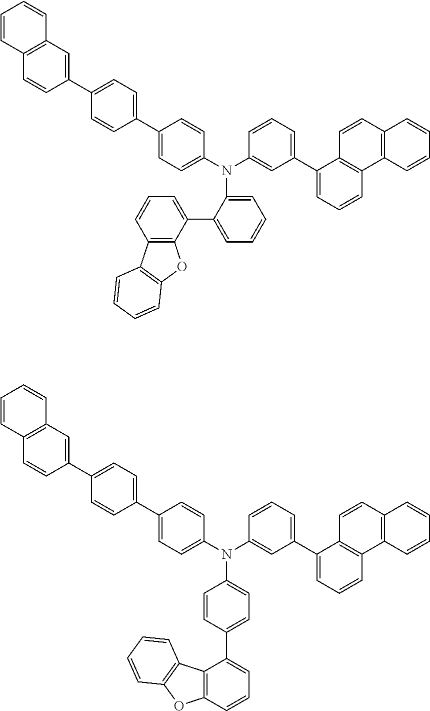 [SMILES: c1cc(-c2cccc3c2ccc2ccccc23)cc(N(c2ccc(-c3ccc(-c4ccc5ccccc5c4)cc3)cc2)c2ccccc2-c2cccc3c2oc2ccccc23)c1 ; c1cc(-c2cccc3c2ccc2ccccc23)cc(N(c2ccc(-c3ccc(-c4ccc5ccccc5c4)cc3)cc2)c2ccc(-c3cccc4oc5ccccc5c34)cc2)c1]

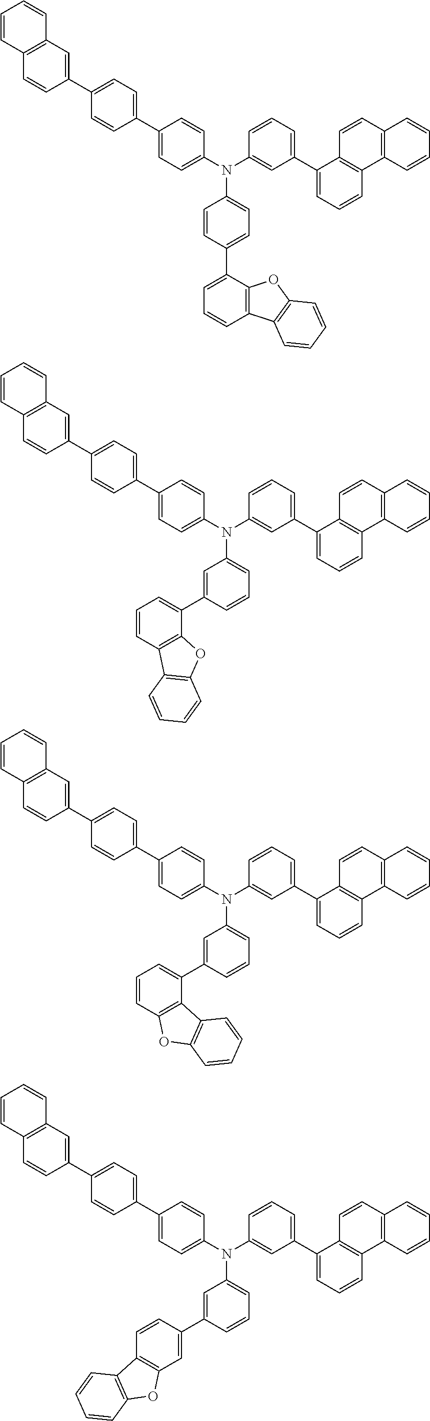 [SMILES: c1cc(-c2cccc3c2ccc2ccccc23)cc(N(c2ccc(-c3ccc(-c4ccc5ccccc5c4)cc3)cc2)c2ccc(-c3cccc4c3oc3ccccc34)cc2)c1 ; c1cc(-c2cccc3c2ccc2ccccc23)cc(N(c2ccc(-c3ccc(-c4ccc5ccccc5c4)cc3)cc2)c2cccc(-c3cccc4c3oc3ccccc34)c2)c1 ; c1cc(-c2cccc3c2ccc2ccccc23)cc(N(c2ccc(-c3ccc(-c4ccc5ccccc5c4)cc3)cc2)c2cccc(-c3cccc4oc5ccccc5c34)c2)c1 ; c1cc(-c2ccc3c(c2)oc2ccccc23)cc(N(c2ccc(-c3ccc(-c4ccc5ccccc5c4)cc3)cc2)c2cccc(-c3cccc4c3ccc3ccccc34)c2)c1]

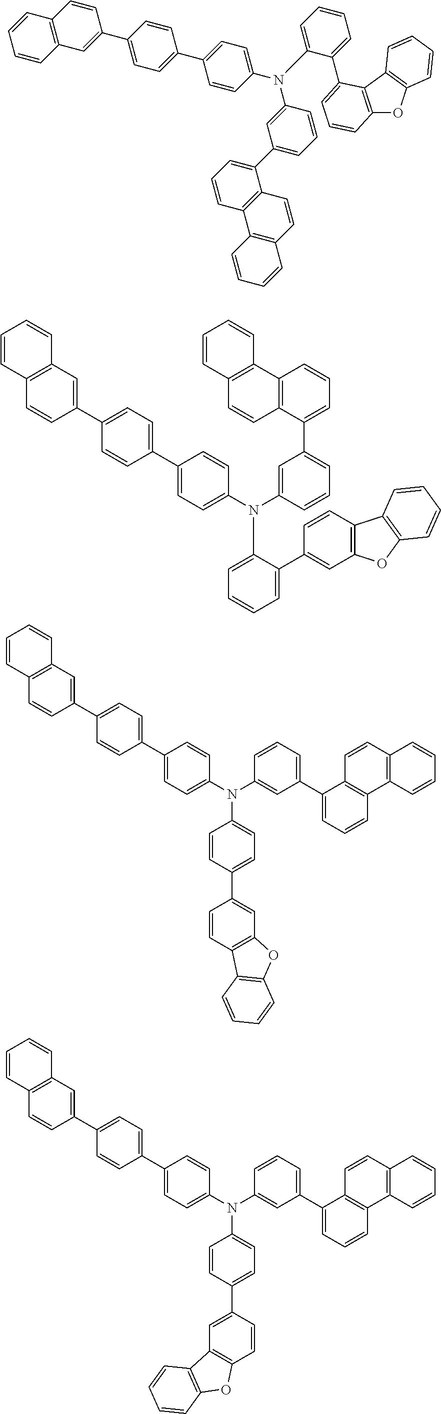 [SMILES: c1cc(-c2cccc3c2ccc2ccccc23)cc(N(c2ccc(-c3ccc(-c4ccc5ccccc5c4)cc3)cc2)c2ccccc2-c2cccc3oc4ccccc4c23)c1 ; c1cc(-c2cccc3c2ccc2ccccc23)cc(N(c2ccc(-c3ccc(-c4ccc5ccccc5c4)cc3)cc2)c2ccccc2-c2ccc3c(c2)oc2ccccc23)c1 ; c1cc(-c2cccc3c2ccc2ccccc23)cc(N(c2ccc(-c3ccc(-c4ccc5ccccc5c4)cc3)cc2)c2ccc(-c3ccc4c(c3)oc3ccccc34)cc2)c1 ; c1cc(-c2cccc3c2ccc2ccccc23)cc(N(c2ccc(-c3ccc(-c4ccc5ccccc5c4)cc3)cc2)c2ccc(-c3ccc4oc5ccccc5c4c3)cc2)c1]

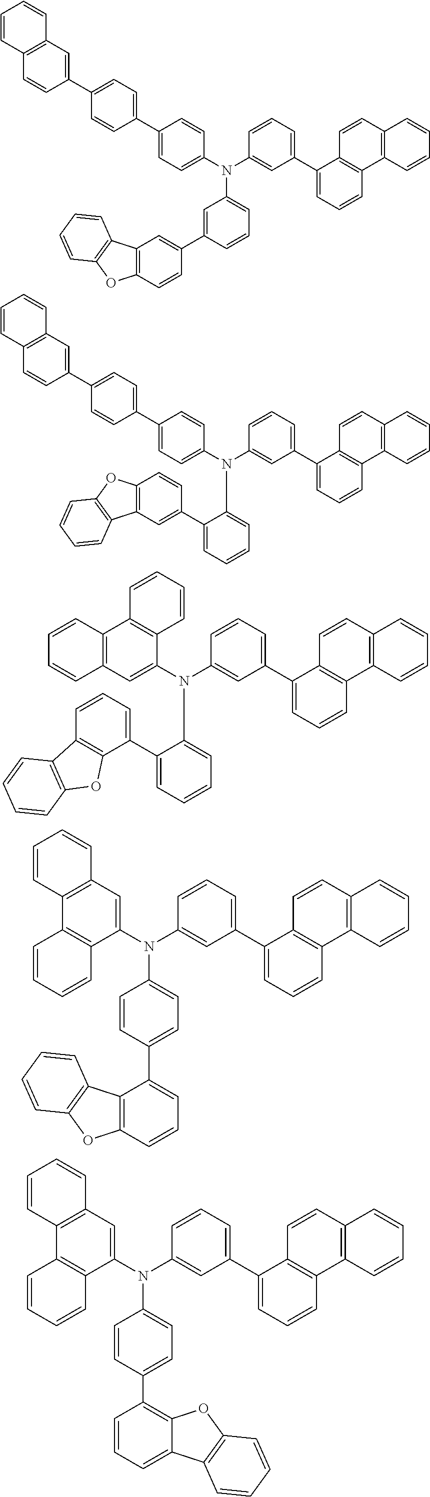

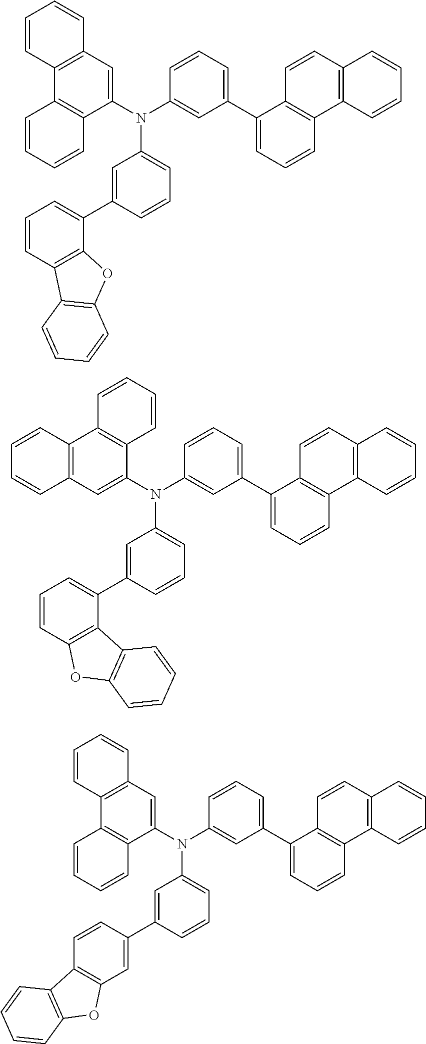

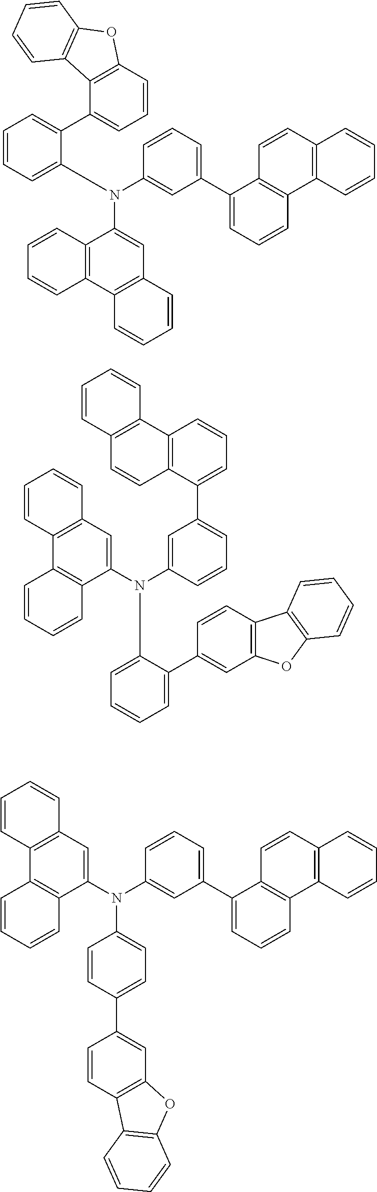

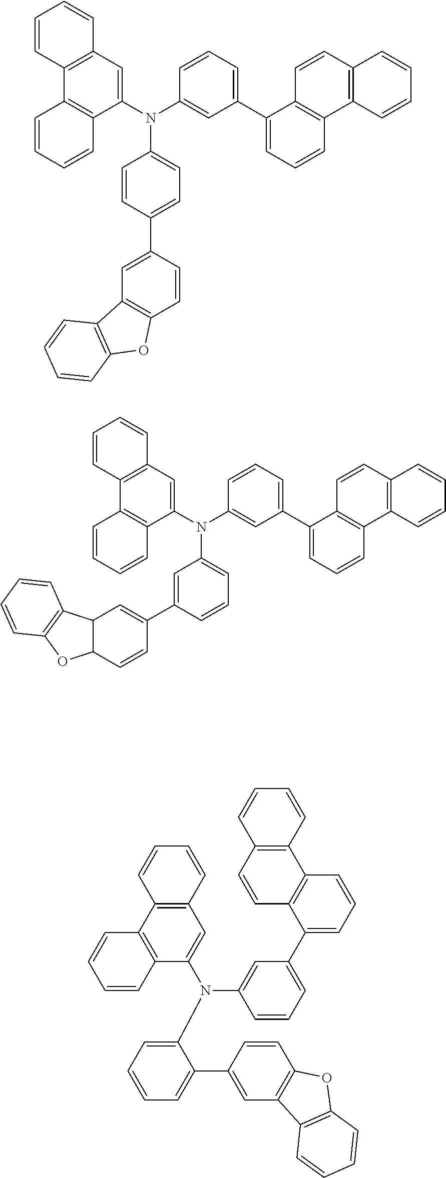

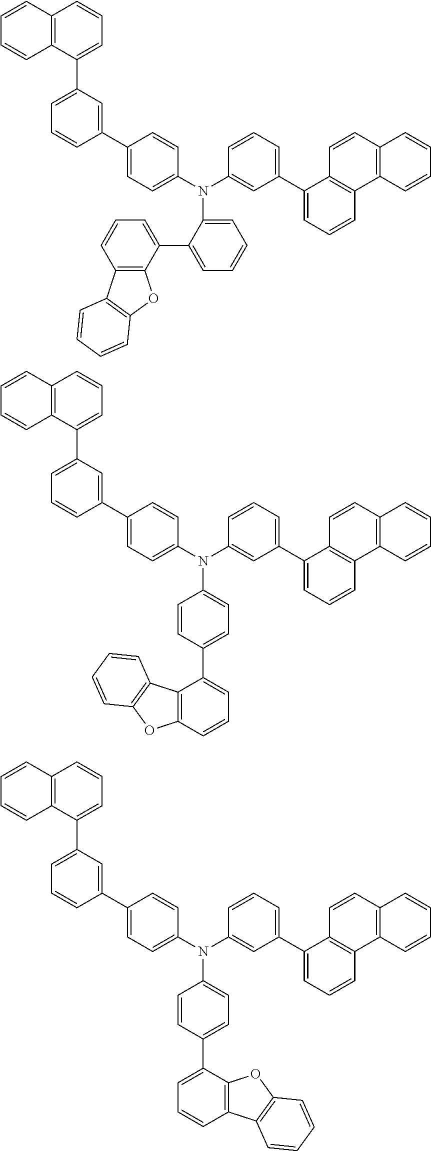

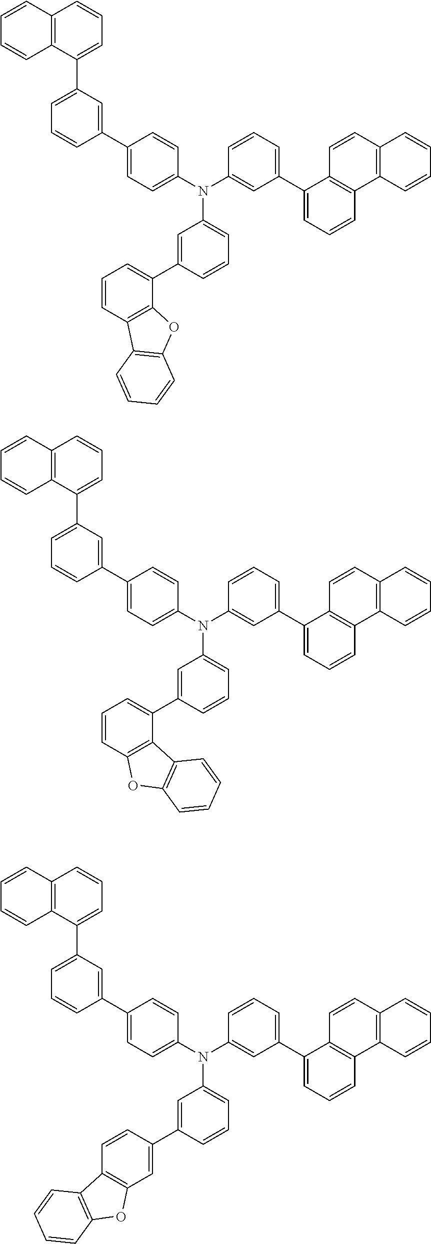 [SMILES: c1cc(-c2ccc(N(c3cccc(-c4cccc5c4ccc4ccccc45)c3)c3cccc(-c4cccc5c4oc4ccccc45)c3)cc2)cc(-c2cccc3ccccc23)c1 ; c1cc(-c2ccc(N(c3cccc(-c4cccc5c4ccc4ccccc45)c3)c3cccc(-c4cccc5oc6ccccc6c45)c3)cc2)cc(-c2cccc3ccccc23)c1 ; c1cc(-c2ccc(N(c3cccc(-c4ccc5c(c4)oc4ccccc45)c3)c3cccc(-c4cccc5c4ccc4ccccc45)c3)cc2)cc(-c2cccc3ccccc23)c1]

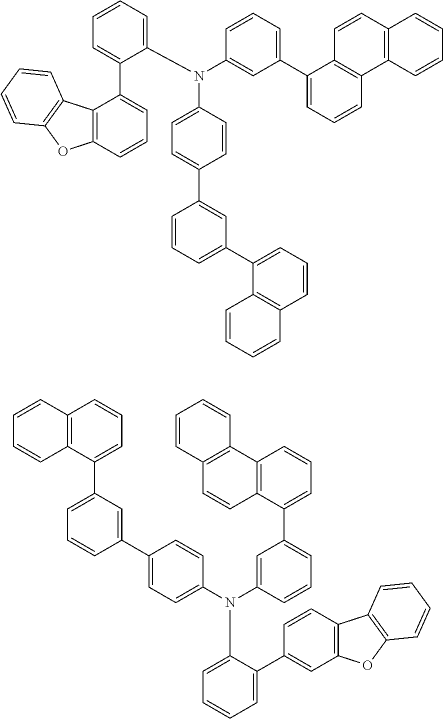 [SMILES: c1cc(-c2ccc(N(c3cccc(-c4cccc5c4ccc4ccccc45)c3)c3ccccc3-c3cccc4oc5ccccc5c34)cc2)cc(-c2cccc3ccccc23)c1 ; c1cc(-c2ccc(N(c3cccc(-c4cccc5c4ccc4ccccc45)c3)c3ccccc3-c3ccc4c(c3)oc3ccccc34)cc2)cc(-c2cccc3ccccc23)c1]

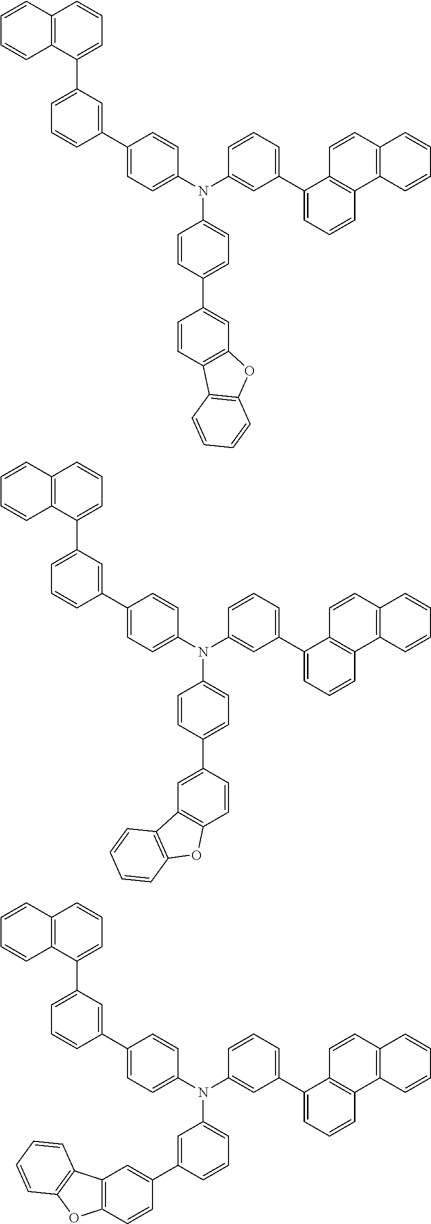 [SMILES: c1cc(-c2ccc(N(c3ccc(-c4ccc5c(c4)oc4ccccc45)cc3)c3cccc(-c4cccc5c4ccc4ccccc45)c3)cc2)cc(-c2cccc3ccccc23)c1 ; c1cc(-c2ccc(N(c3ccc(-c4ccc5oc6ccccc6c5c4)cc3)c3cccc(-c4cccc5c4ccc4ccccc45)c3)cc2)cc(-c2cccc3ccccc23)c1 ; c1cc(-c2ccc(N(c3cccc(-c4ccc5oc6ccccc6c5c4)c3)c3cccc(-c4cccc5c4ccc4ccccc45)c3)cc2)cc(-c2cccc3ccccc23)c1]

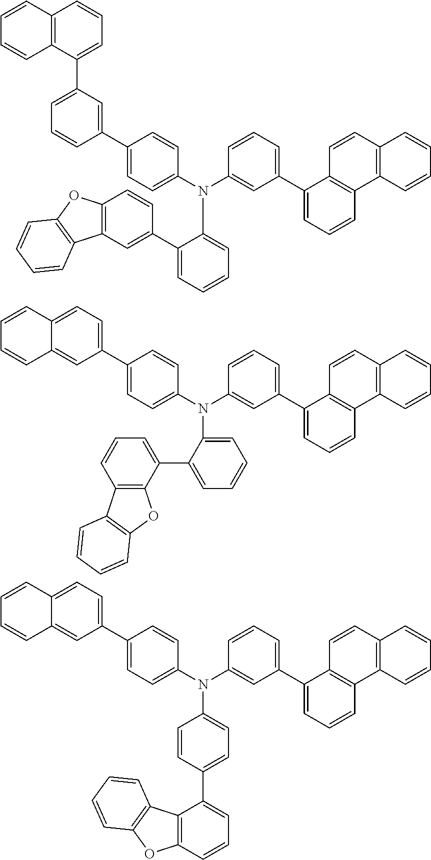

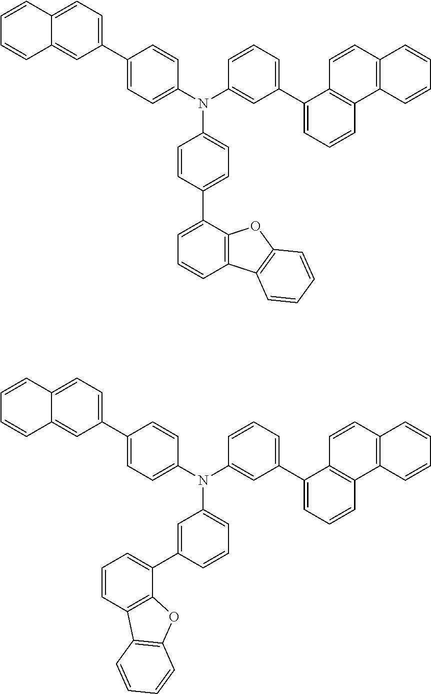 [SMILES: c1cc(-c2cccc3c2ccc2ccccc23)cc(N(c2ccc(-c3ccc4ccccc4c3)cc2)c2ccc(-c3cccc4c3oc3ccccc34)cc2)c1 ; c1cc(-c2cccc3c2ccc2ccccc23)cc(N(c2ccc(-c3ccc4ccccc4c3)cc2)c2cccc(-c3cccc4c3oc3ccccc34)c2)c1]

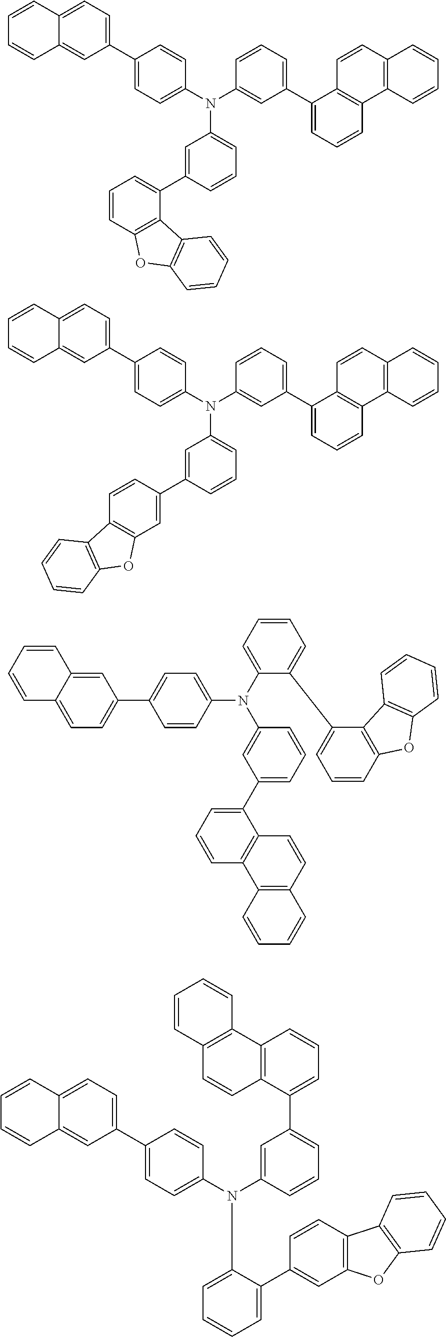 [SMILES: c1cc(-c2cccc3c2ccc2ccccc23)cc(N(c2ccc(-c3ccc4ccccc4c3)cc2)c2cccc(-c3cccc4oc5ccccc5c34)c2)c1 ; c1cc(-c2ccc3c(c2)oc2ccccc23)cc(N(c2ccc(-c3ccc4ccccc4c3)cc2)c2cccc(-c3cccc4c3ccc3ccccc34)c2)c1 ; c1cc(-c2cccc3c2ccc2ccccc23)cc(N(c2ccc(-c3ccc4ccccc4c3)cc2)c2ccccc2-c2cccc3oc4ccccc4c23)c1 ; c1cc(-c2cccc3c2ccc2ccccc23)cc(N(c2ccc(-c3ccc4ccccc4c3)cc2)c2ccccc2-c2ccc3c(c2)oc2ccccc23)c1]

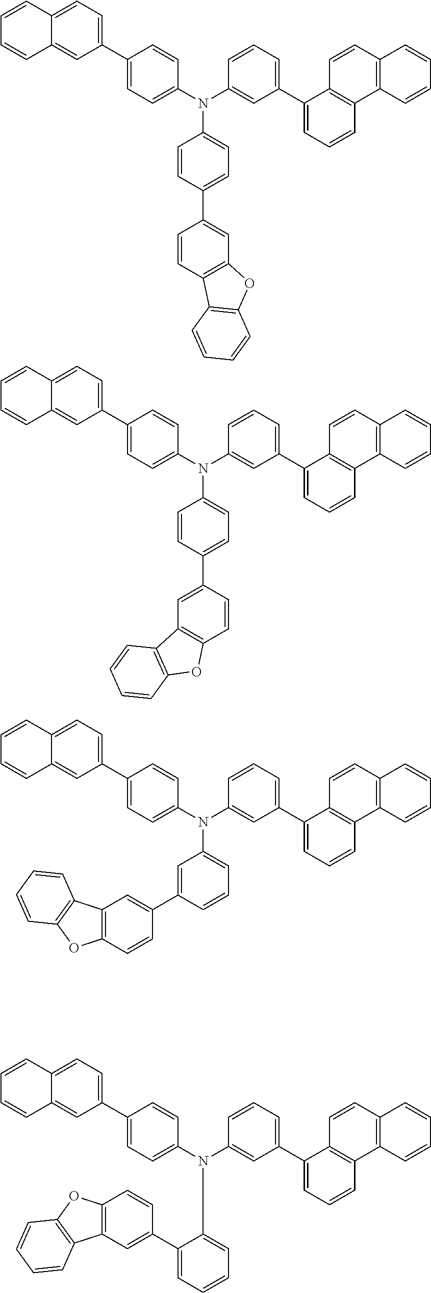 [SMILES: c1cc(-c2cccc3c2ccc2ccccc23)cc(N(c2ccc(-c3ccc4ccccc4c3)cc2)c2ccc(-c3ccc4c(c3)oc3ccccc34)cc2)c1 ; c1cc(-c2cccc3c2ccc2ccccc23)cc(N(c2ccc(-c3ccc4ccccc4c3)cc2)c2ccc(-c3ccc4oc5ccccc5c4c3)cc2)c1 ; c1cc(-c2ccc3oc4ccccc4c3c2)cc(N(c2ccc(-c3ccc4ccccc4c3)cc2)c2cccc(-c3cccc4c3ccc3ccccc34)c2)c1 ; c1cc(-c2cccc3c2ccc2ccccc23)cc(N(c2ccc(-c3ccc4ccccc4c3)cc2)c2ccccc2-c2ccc3oc4ccccc4c3c2)c1]

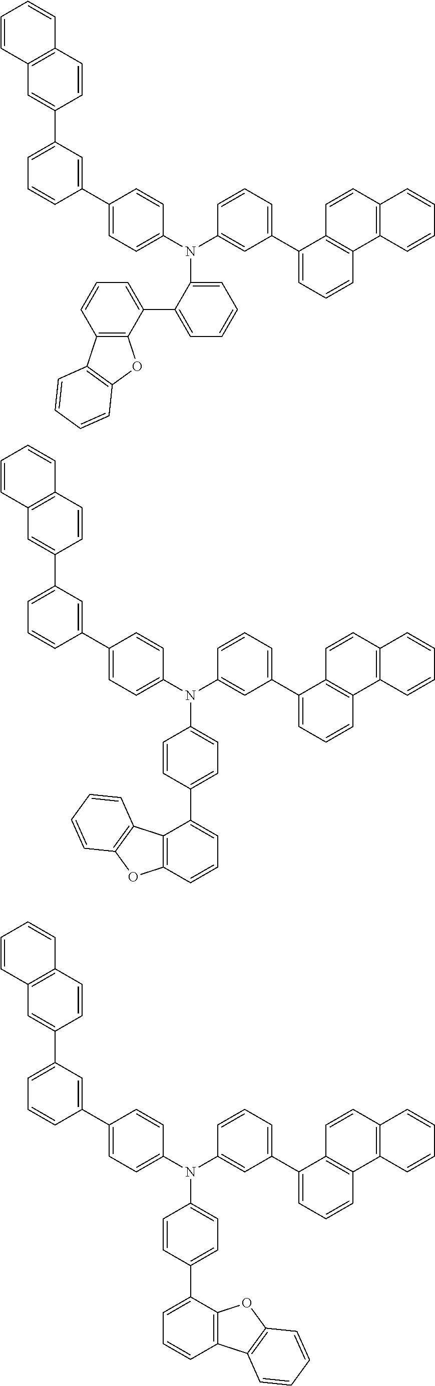

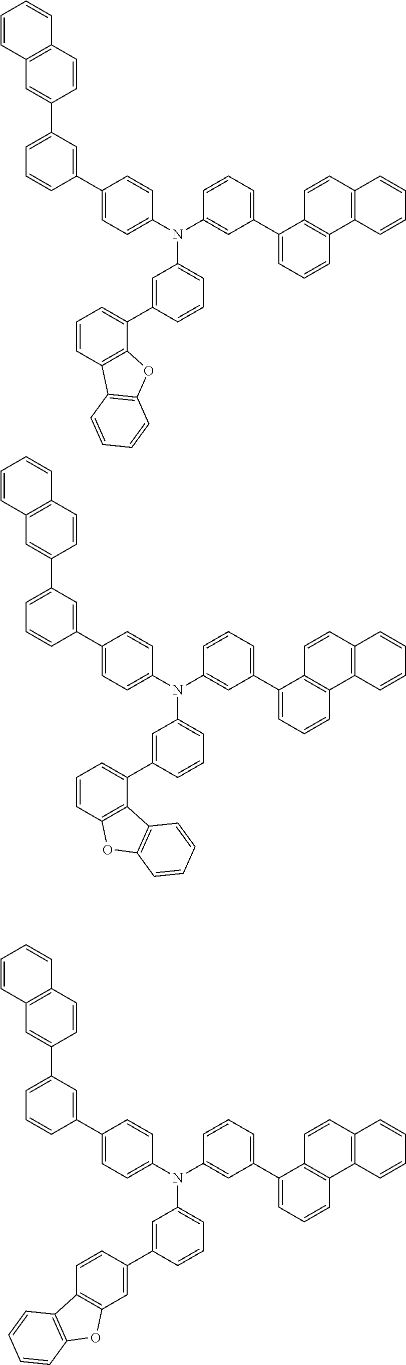 [SMILES: c1cc(-c2ccc(N(c3cccc(-c4cccc5c4ccc4ccccc45)c3)c3cccc(-c4cccc5c4oc4ccccc45)c3)cc2)cc(-c2ccc3ccccc3c2)c1 ; c1cc(-c2ccc(N(c3cccc(-c4cccc5c4ccc4ccccc45)c3)c3cccc(-c4cccc5oc6ccccc6c45)c3)cc2)cc(-c2ccc3ccccc3c2)c1 ; c1cc(-c2ccc(N(c3cccc(-c4ccc5c(c4)oc4ccccc45)c3)c3cccc(-c4cccc5c4ccc4ccccc45)c3)cc2)cc(-c2ccc3ccccc3c2)c1]

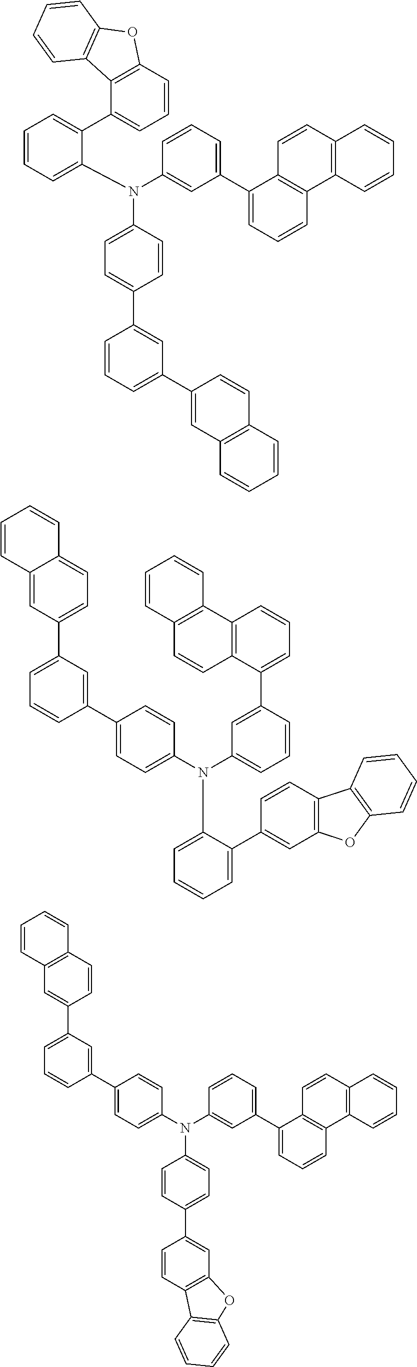 [SMILES: c1cc(-c2ccc(N(c3cccc(-c4cccc5c4ccc4ccccc45)c3)c3ccccc3-c3cccc4oc5ccccc5c34)cc2)cc(-c2ccc3ccccc3c2)c1 ; c1cc(-c2ccc(N(c3cccc(-c4cccc5c4ccc4ccccc45)c3)c3ccccc3-c3ccc4c(c3)oc3ccccc34)cc2)cc(-c2ccc3ccccc3c2)c1 ; c1cc(-c2ccc(N(c3ccc(-c4ccc5c(c4)oc4ccccc45)cc3)c3cccc(-c4cccc5c4ccc4ccccc45)c3)cc2)cc(-c2ccc3ccccc3c2)c1]

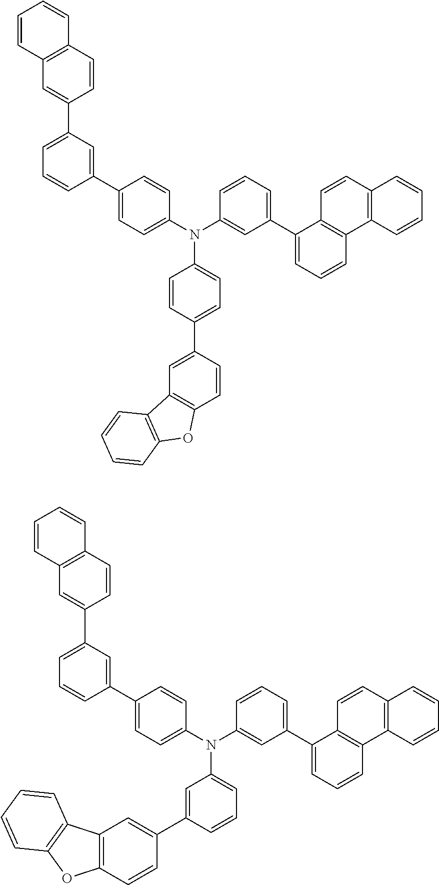 [SMILES: c1cc(-c2ccc(N(c3ccc(-c4ccc5oc6ccccc6c5c4)cc3)c3cccc(-c4cccc5c4ccc4ccccc45)c3)cc2)cc(-c2ccc3ccccc3c2)c1 ; c1cc(-c2ccc(N(c3cccc(-c4ccc5oc6ccccc6c5c4)c3)c3cccc(-c4cccc5c4ccc4ccccc45)c3)cc2)cc(-c2ccc3ccccc3c2)c1]

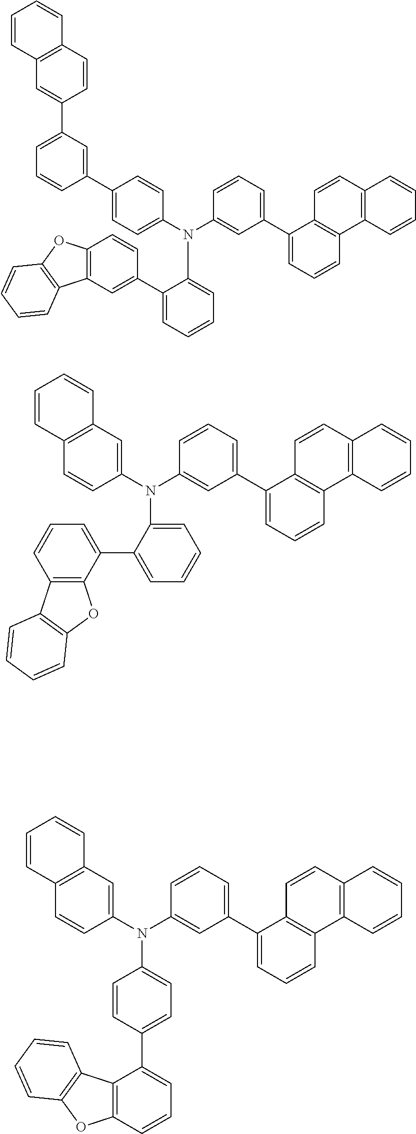 [SMILES: c1cc(-c2ccc(N(c3cccc(-c4cccc5c4ccc4ccccc45)c3)c3ccccc3-c3ccc4oc5ccccc5c4c3)cc2)cc(-c2ccc3ccccc3c2)c1 ; c1cc(-c2cccc3c2ccc2ccccc23)cc(N(c2ccc3ccccc3c2)c2ccccc2-c2cccc3c2oc2ccccc23)c1 ; c1cc(-c2cccc3c2ccc2ccccc23)cc(N(c2ccc(-c3cccc4oc5ccccc5c34)cc2)c2ccc3ccccc3c2)c1]

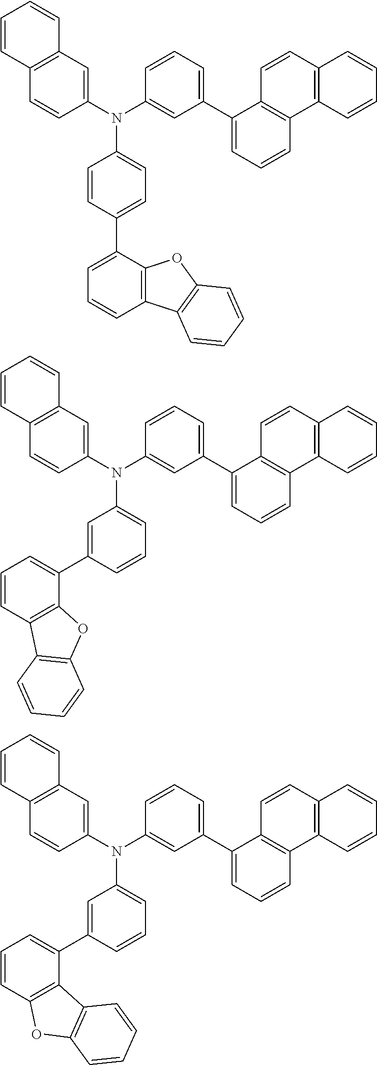

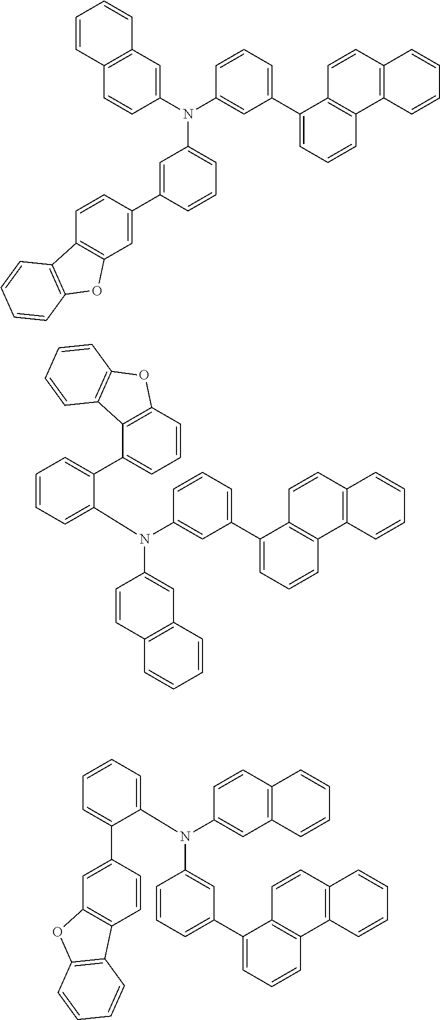

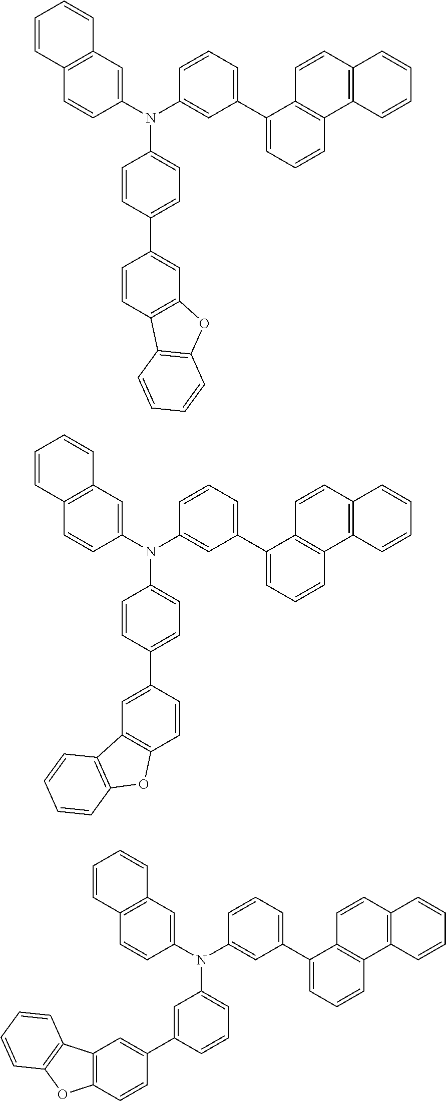

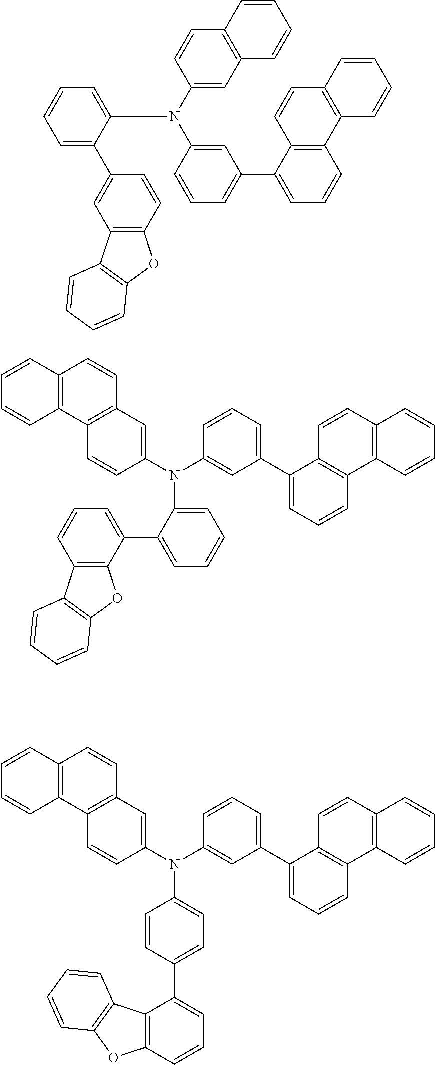

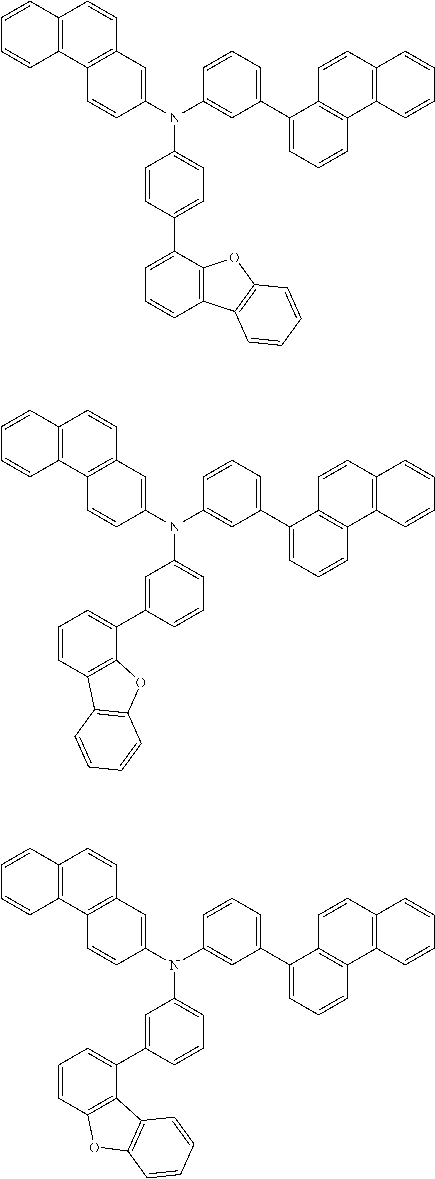 [SMILES: c1cc(-c2cccc3c2ccc2ccccc23)cc(N(c2ccc(-c3cccc4c3oc3ccccc34)cc2)c2ccc3c(ccc4ccccc43)c2)c1 ; c1cc(-c2cccc3c2ccc2ccccc23)cc(N(c2cccc(-c3cccc4c3oc3ccccc34)c2)c2ccc3c(ccc4ccccc43)c2)c1 ; c1cc(-c2cccc3c2ccc2ccccc23)cc(N(c2cccc(-c3cccc4oc5ccccc5c34)c2)c2ccc3c(ccc4ccccc43)c2)c1]

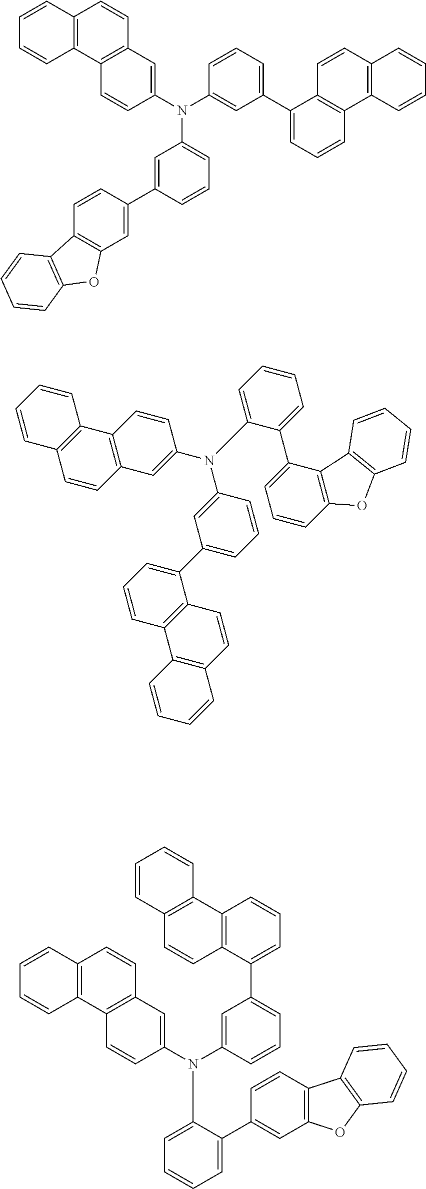 [SMILES: c1cc(-c2ccc3c(c2)oc2ccccc23)cc(N(c2cccc(-c3cccc4c3ccc3ccccc34)c2)c2ccc3c(ccc4ccccc43)c2)c1 ; c1cc(-c2cccc3c2ccc2ccccc23)cc(N(c2ccc3c(ccc4ccccc43)c2)c2ccccc2-c2cccc3oc4ccccc4c23)c1 ; c1cc(-c2cccc3c2ccc2ccccc23)cc(N(c2ccc3c(ccc4ccccc43)c2)c2ccccc2-c2ccc3c(c2)oc2ccccc23)c1]

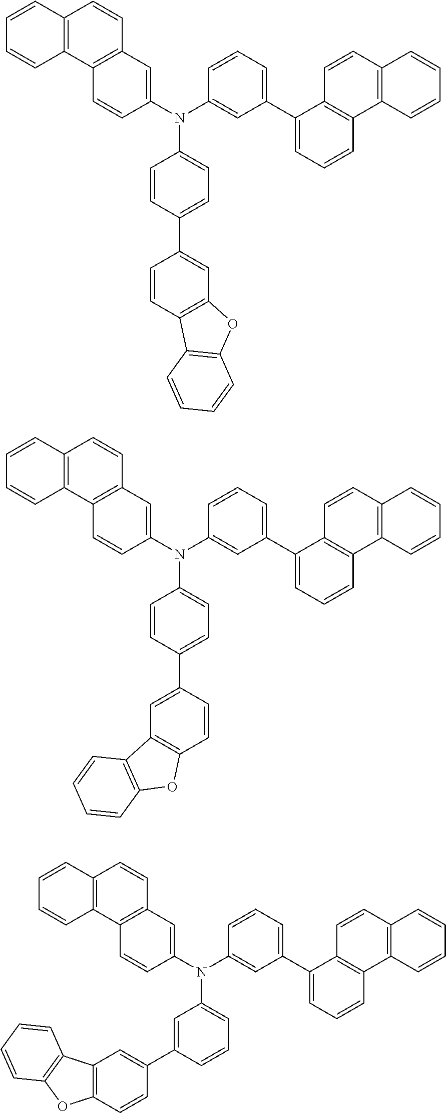

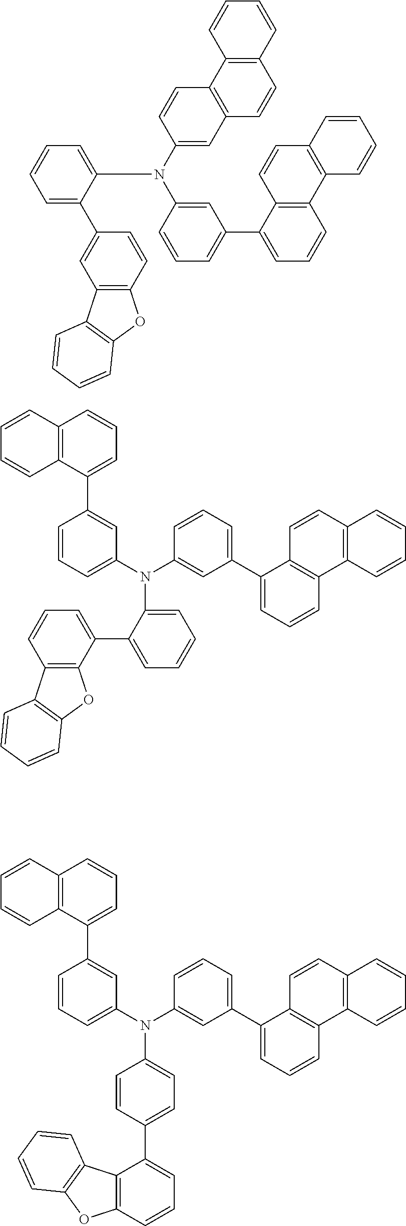

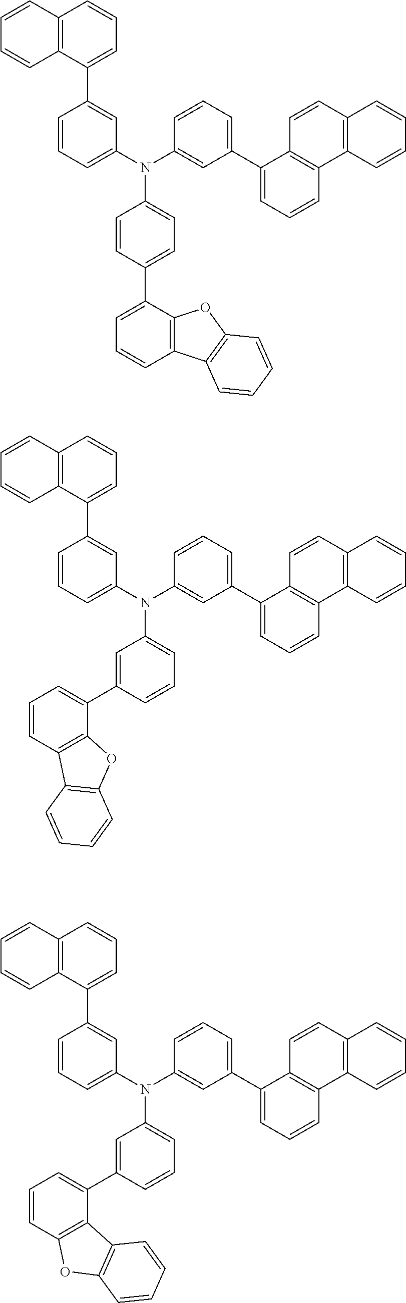 [SMILES: c1cc(-c2cccc3ccccc23)cc(N(c2ccc(-c3cccc4c3oc3ccccc34)cc2)c2cccc(-c3cccc4c3ccc3ccccc34)c2)c1 ; c1cc(-c2cccc3ccccc23)cc(N(c2cccc(-c3cccc4c3ccc3ccccc34)c2)c2cccc(-c3cccc4c3oc3ccccc34)c2)c1 ; c1cc(-c2cccc3ccccc23)cc(N(c2cccc(-c3cccc4c3ccc3ccccc34)c2)c2cccc(-c3cccc4oc5ccccc5c34)c2)c1]

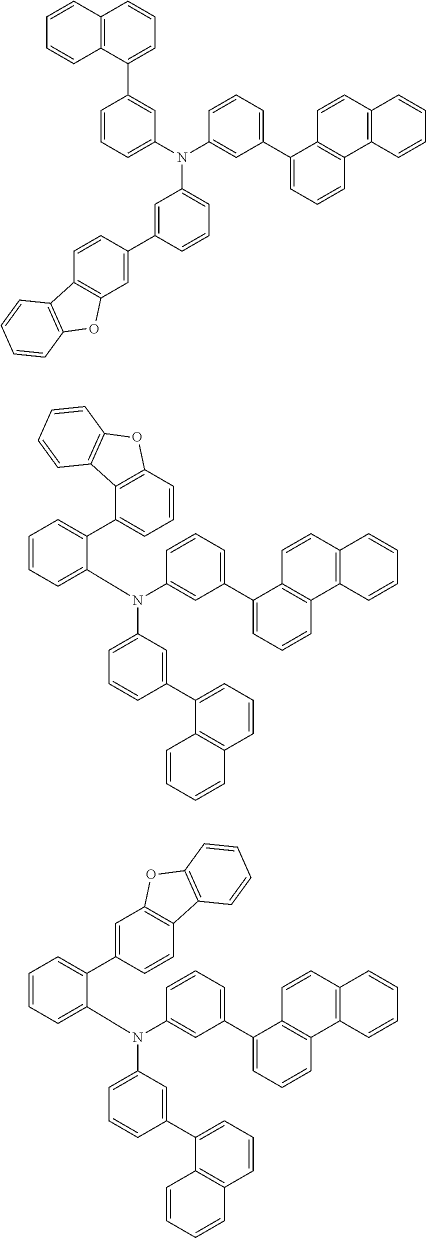

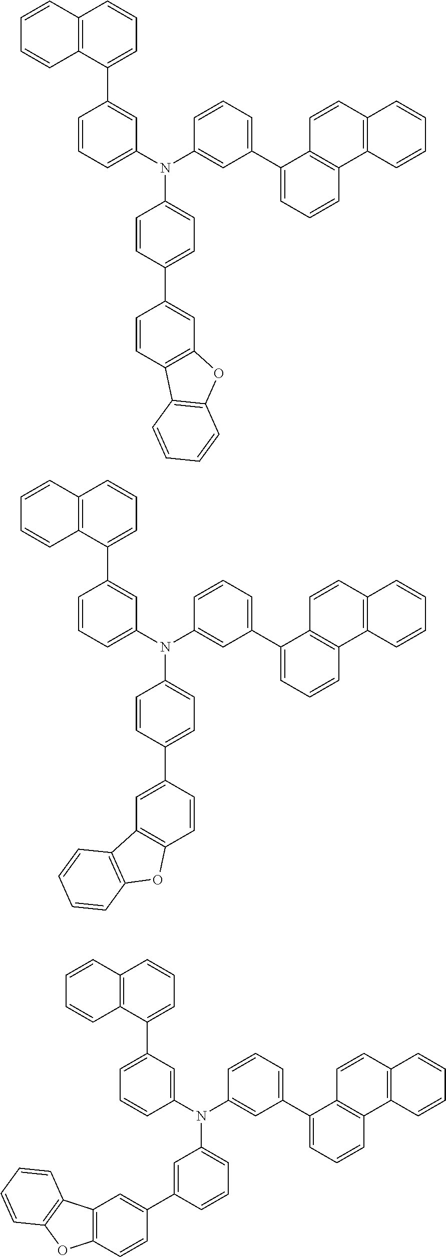 [SMILES: c1cc(-c2cccc3ccccc23)cc(N(c2ccc(-c3ccc4c(c3)oc3ccccc34)cc2)c2cccc(-c3cccc4c3ccc3ccccc34)c2)c1 ; c1cc(-c2cccc3ccccc23)cc(N(c2ccc(-c3ccc4oc5ccccc5c4c3)cc2)c2cccc(-c3cccc4c3ccc3ccccc34)c2)c1 ; c1cc(-c2ccc3oc4ccccc4c3c2)cc(N(c2cccc(-c3cccc4ccccc34)c2)c2cccc(-c3cccc4c3ccc3ccccc34)c2)c1]

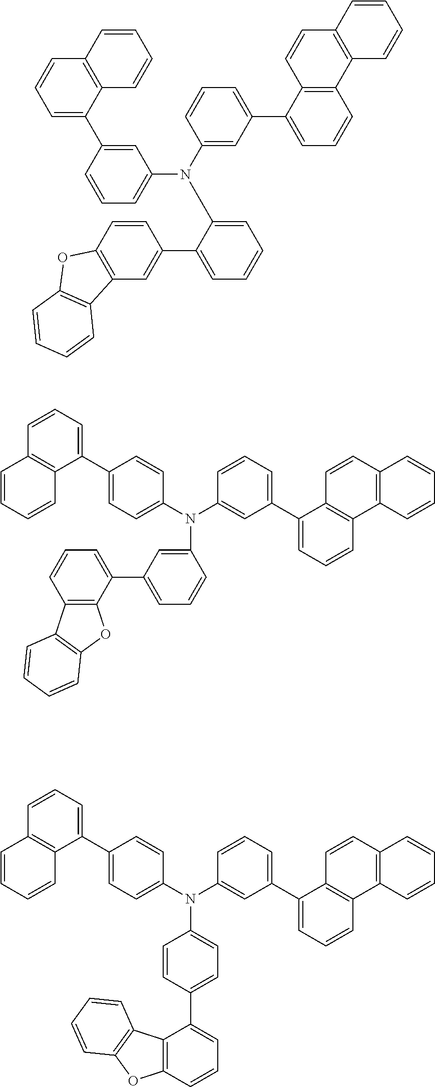 [SMILES: c1cc(-c2cccc3ccccc23)cc(N(c2cccc(-c3cccc4c3ccc3ccccc34)c2)c2ccccc2-c2ccc3oc4ccccc4c3c2)c1 ; c1cc(-c2cccc3c2ccc2ccccc23)cc(N(c2ccc(-c3cccc4ccccc34)cc2)c2cccc(-c3cccc4c3oc3ccccc34)c2)c1 ; c1cc(-c2cccc3c2ccc2ccccc23)cc(N(c2ccc(-c3cccc4ccccc34)cc2)c2ccc(-c3cccc4oc5ccccc5c34)cc2)c1]

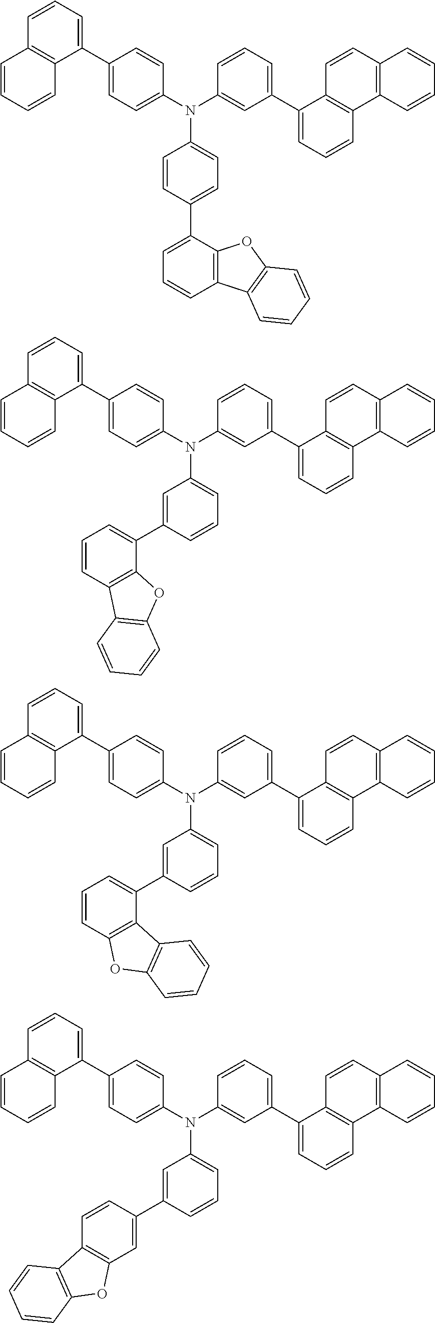

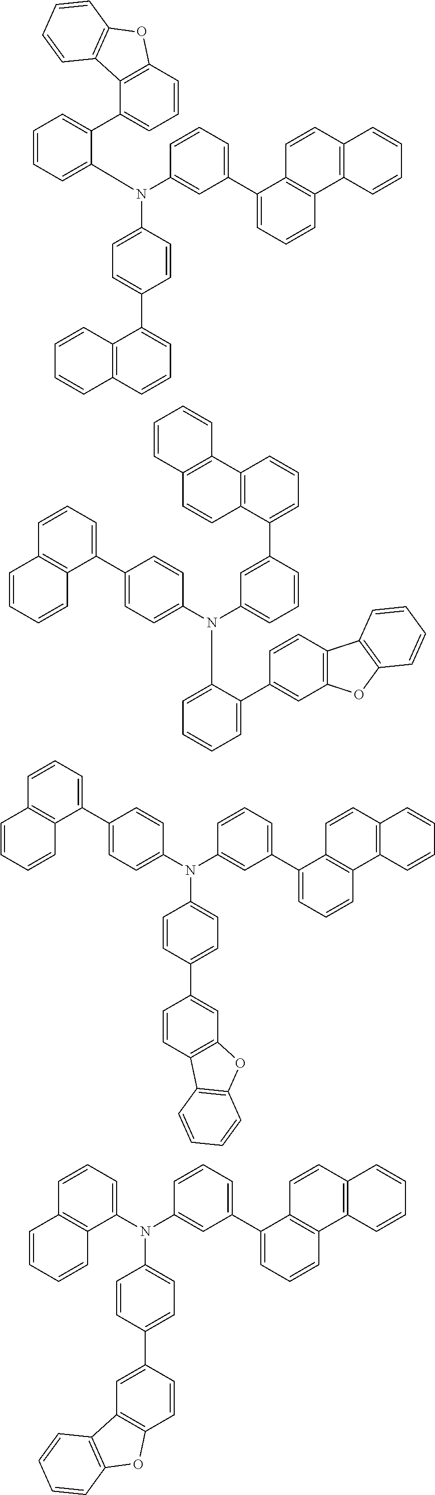

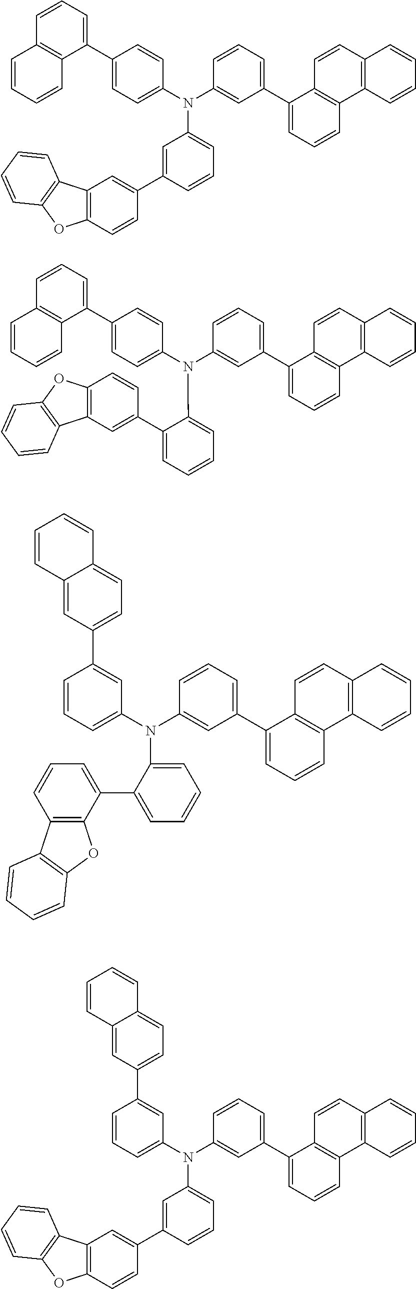

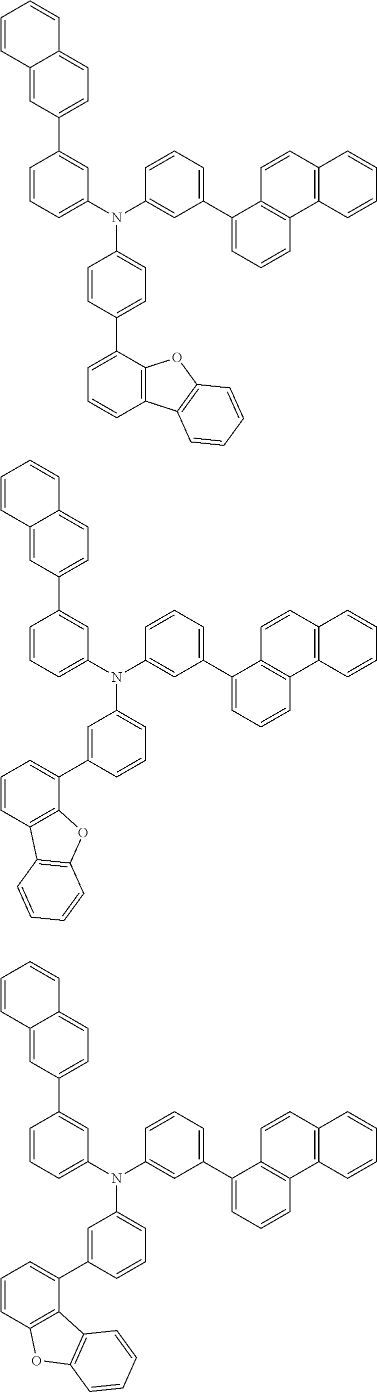

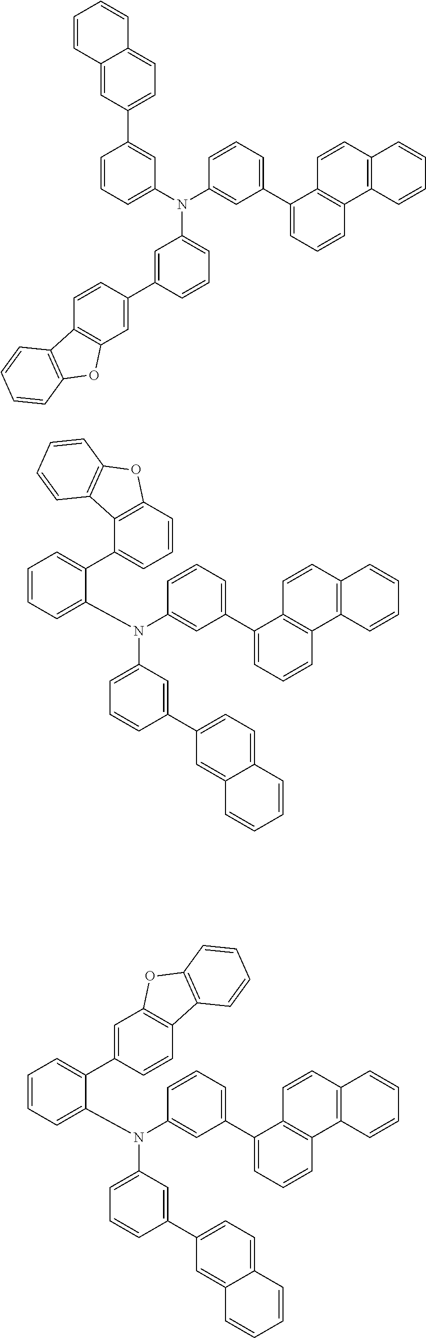

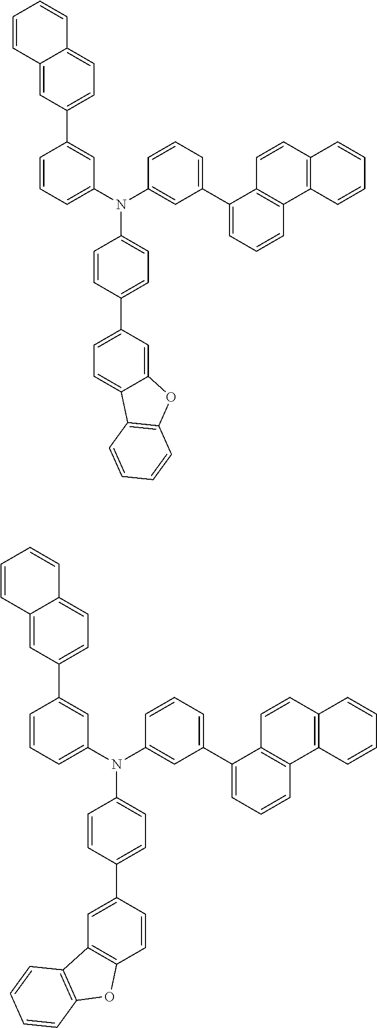 [SMILES: c1cc(-c2ccc3ccccc3c2)cc(N(c2ccc(-c3ccc4c(c3)oc3ccccc34)cc2)c2cccc(-c3cccc4c3ccc3ccccc34)c2)c1 ; c1cc(-c2ccc3ccccc3c2)cc(N(c2ccc(-c3ccc4oc5ccccc5c4c3)cc2)c2cccc(-c3cccc4c3ccc3ccccc34)c2)c1]

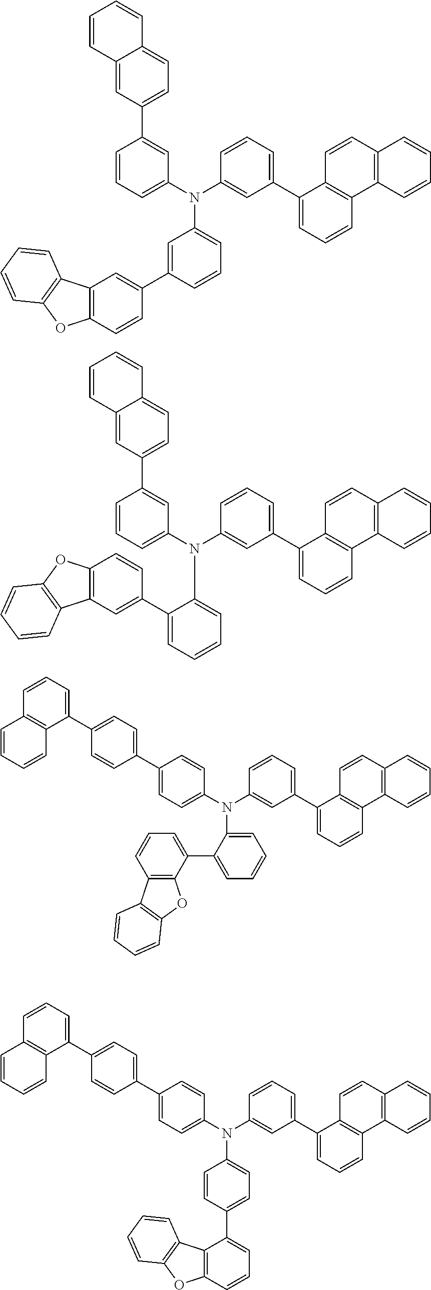

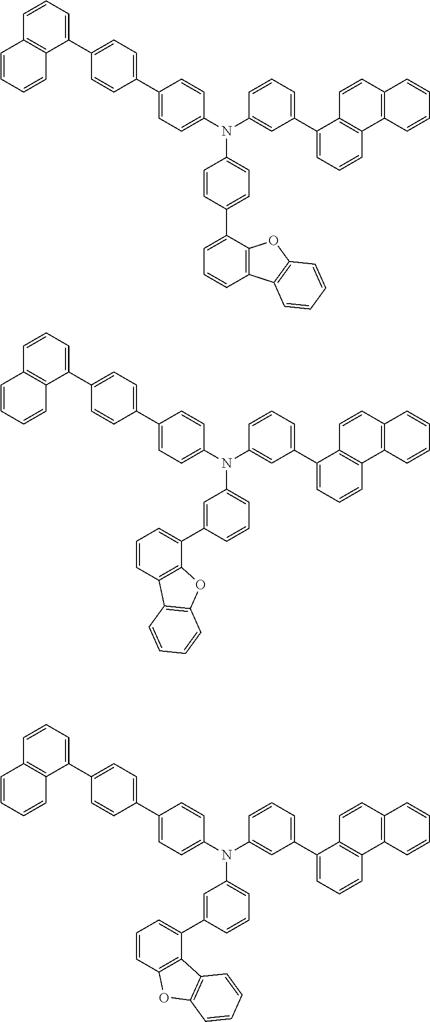 [SMILES: c1cc(-c2cccc3c2ccc2ccccc23)cc(N(c2ccc(-c3ccc(-c4cccc5ccccc45)cc3)cc2)c2ccc(-c3cccc4c3oc3ccccc34)cc2)c1 ; c1cc(-c2cccc3c2ccc2ccccc23)cc(N(c2ccc(-c3ccc(-c4cccc5ccccc45)cc3)cc2)c2cccc(-c3cccc4c3oc3ccccc34)c2)c1 ; c1cc(-c2cccc3c2ccc2ccccc23)cc(N(c2ccc(-c3ccc(-c4cccc5ccccc45)cc3)cc2)c2cccc(-c3cccc4oc5ccccc5c34)c2)c1]

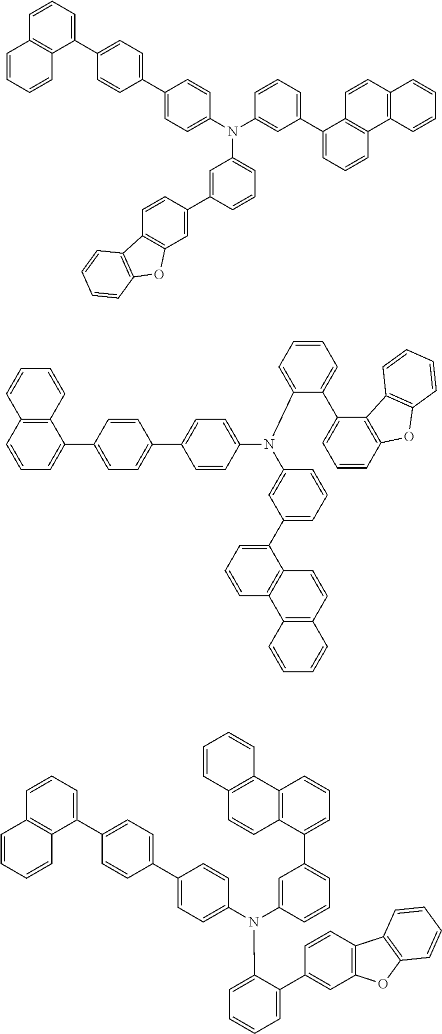 [SMILES: c1cc(-c2ccc3c(c2)oc2ccccc23)cc(N(c2ccc(-c3ccc(-c4cccc5ccccc45)cc3)cc2)c2cccc(-c3cccc4c3ccc3ccccc34)c2)c1 ; c1cc(-c2cccc3c2ccc2ccccc23)cc(N(c2ccc(-c3ccc(-c4cccc5ccccc45)cc3)cc2)c2ccccc2-c2cccc3oc4ccccc4c23)c1 ; c1cc(-c2cccc3c2ccc2ccccc23)cc(N(c2ccc(-c3ccc(-c4cccc5ccccc45)cc3)cc2)c2ccccc2-c2ccc3c(c2)oc2ccccc23)c1]

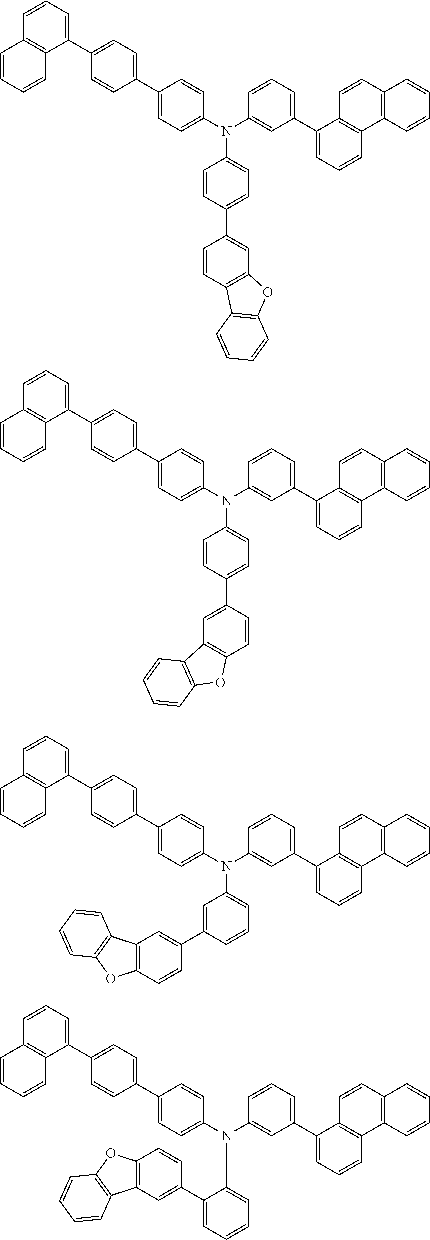 [SMILES: c1cc(-c2cccc3c2ccc2ccccc23)cc(N(c2ccc(-c3ccc(-c4cccc5ccccc45)cc3)cc2)c2ccc(-c3ccc4c(c3)oc3ccccc34)cc2)c1 ; c1cc(-c2cccc3c2ccc2ccccc23)cc(N(c2ccc(-c3ccc(-c4cccc5ccccc45)cc3)cc2)c2ccc(-c3ccc4oc5ccccc5c4c3)cc2)c1 ; c1cc(-c2ccc3oc4ccccc4c3c2)cc(N(c2ccc(-c3ccc(-c4cccc5ccccc45)cc3)cc2)c2cccc(-c3cccc4c3ccc3ccccc34)c2)c1 ; c1cc(-c2cccc3c2ccc2ccccc23)cc(N(c2ccc(-c3ccc(-c4cccc5ccccc45)cc3)cc2)c2ccccc2-c2ccc3oc4ccccc4c3c2)c1]

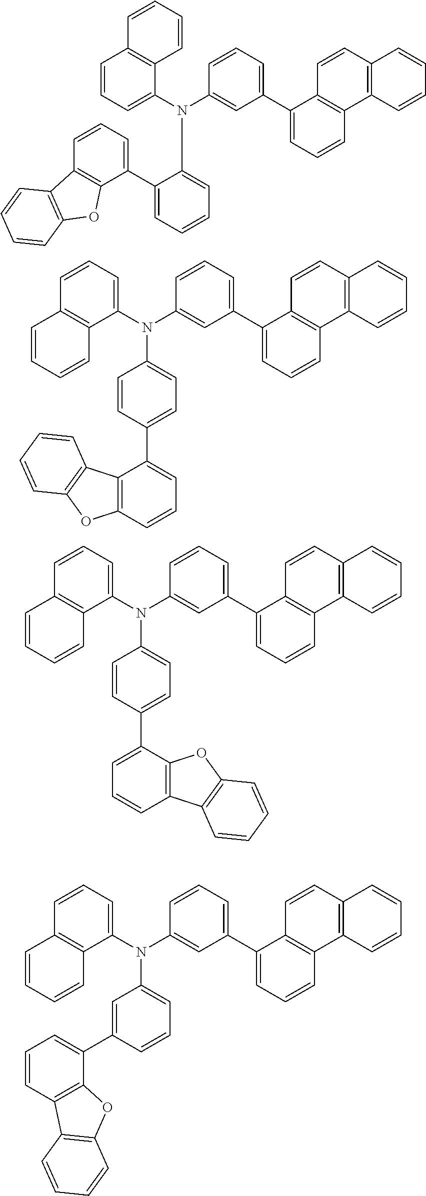

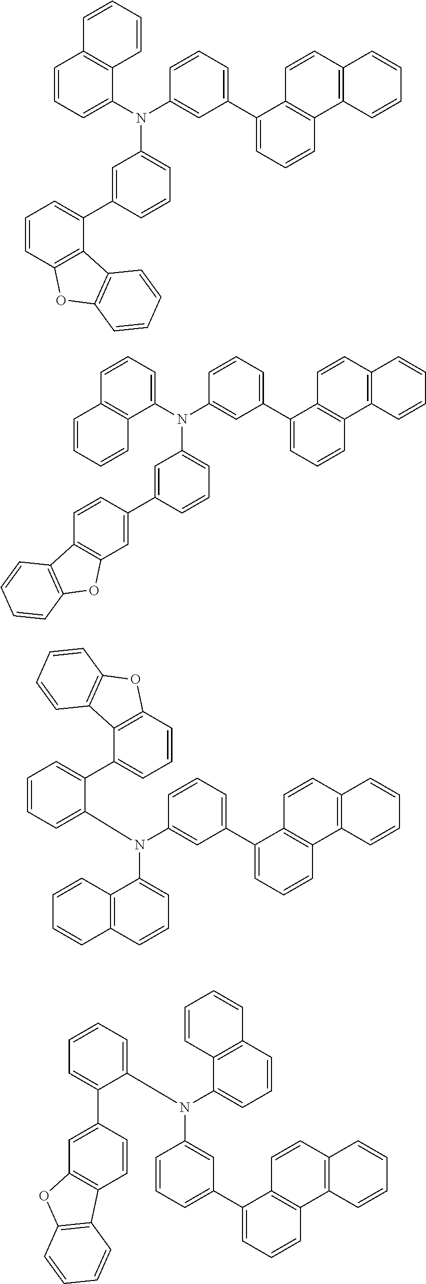

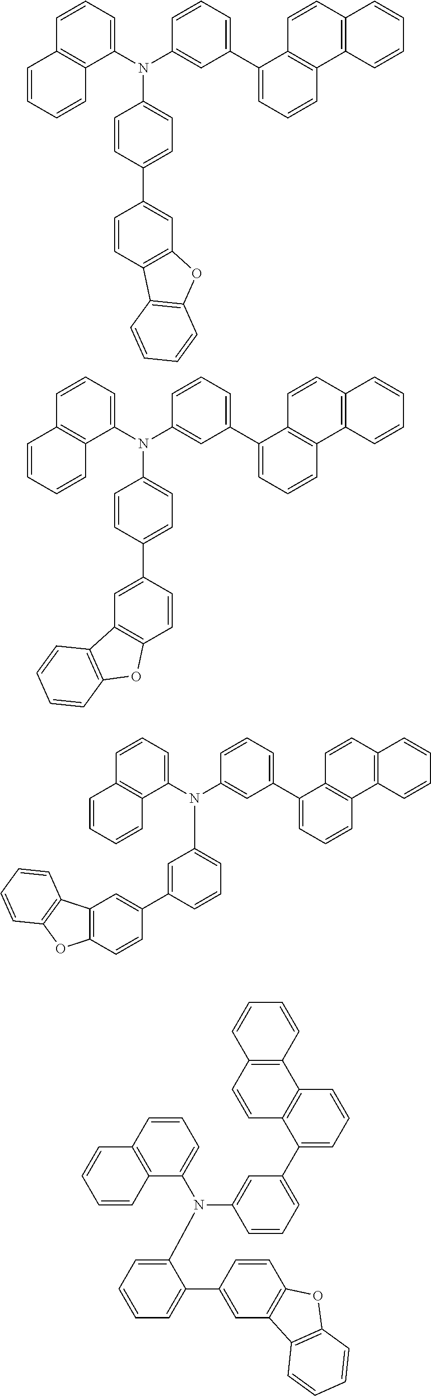

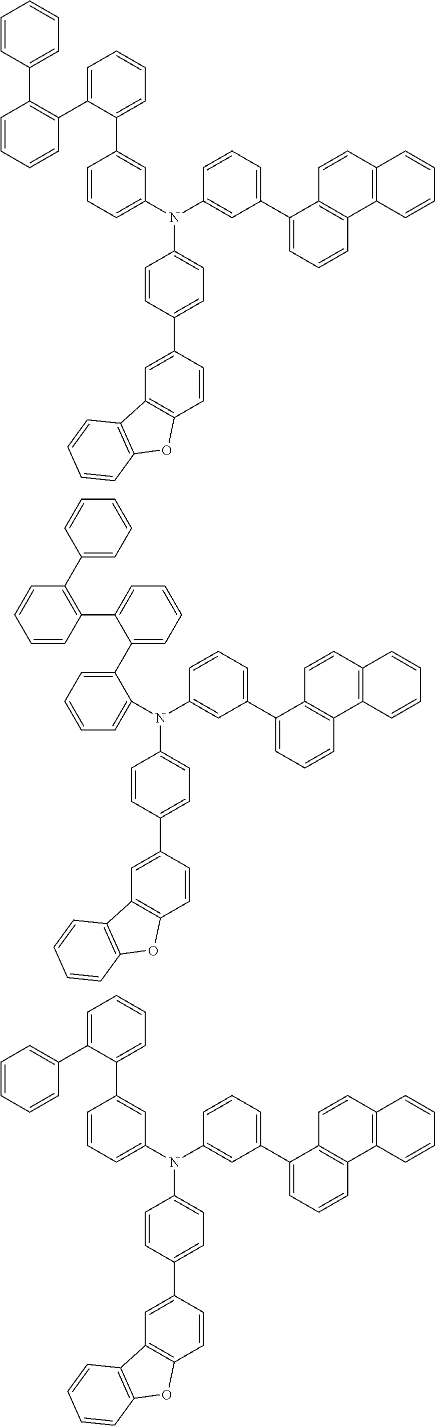

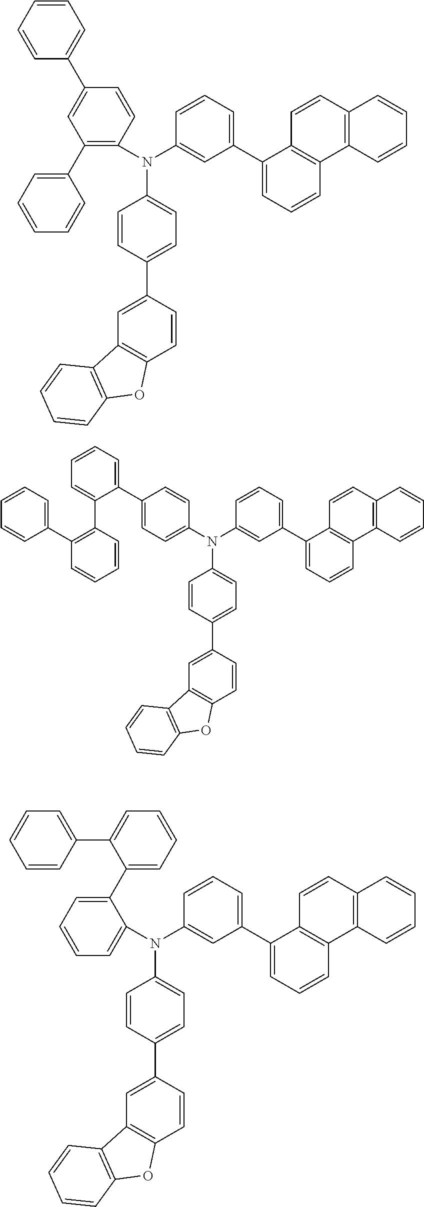

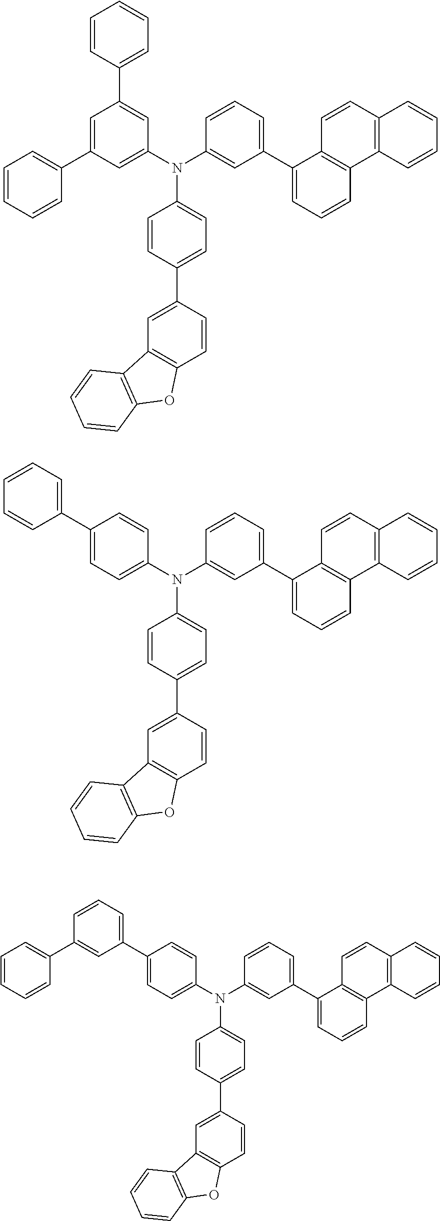 [SMILES: c1ccc(-c2cc(-c3ccccc3)cc(N(c3ccc(-c4ccc5oc6ccccc6c5c4)cc3)c3cccc(-c4cccc5c4ccc4ccccc45)c3)c2)cc1 ; c1ccc(-c2ccc(N(c3ccc(-c4ccc5oc6ccccc6c5c4)cc3)c3cccc(-c4cccc5c4ccc4ccccc45)c3)cc2)cc1 ; c1ccc(-c2cccc(-c3ccc(N(c4ccc(-c5ccc6oc7ccccc7c6c5)cc4)c4cccc(-c5cccc6c5ccc5ccccc56)c4)cc3)c2)cc1]

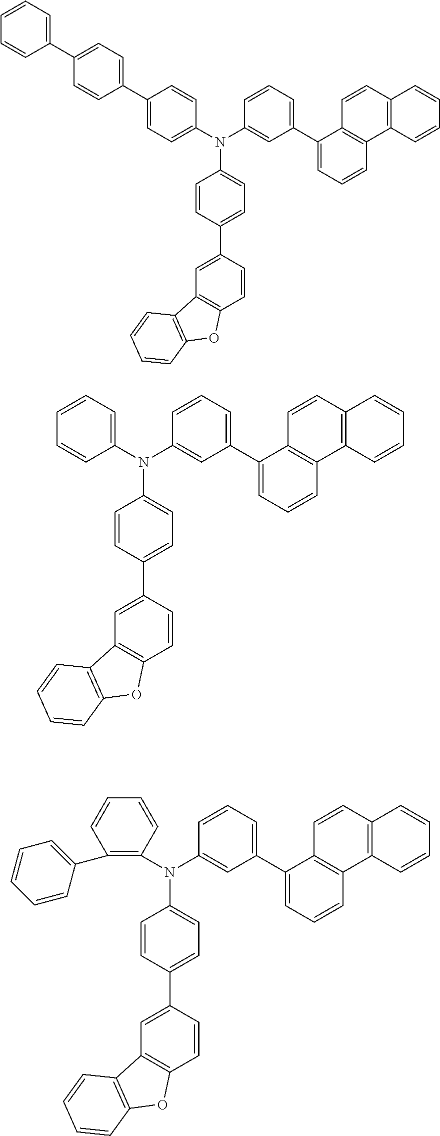

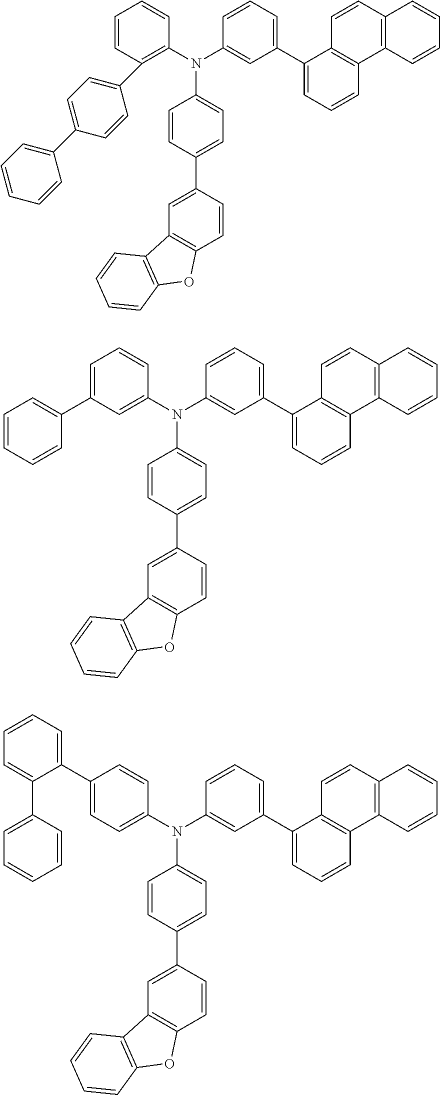

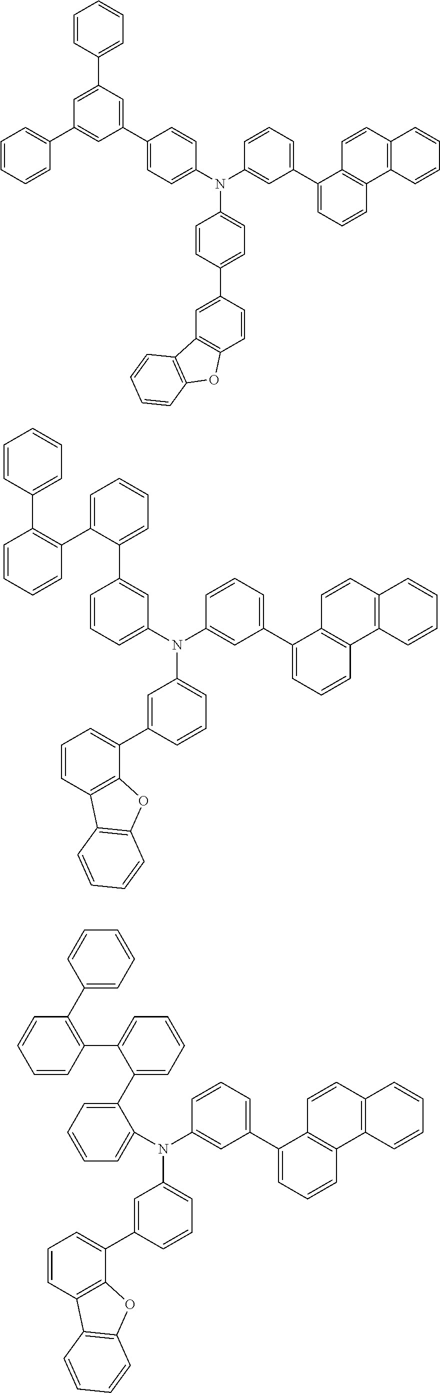

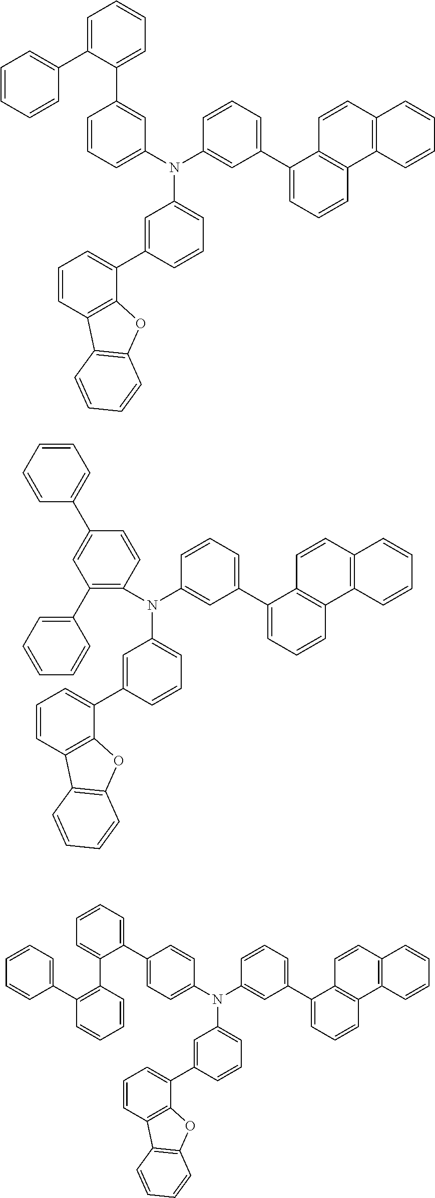

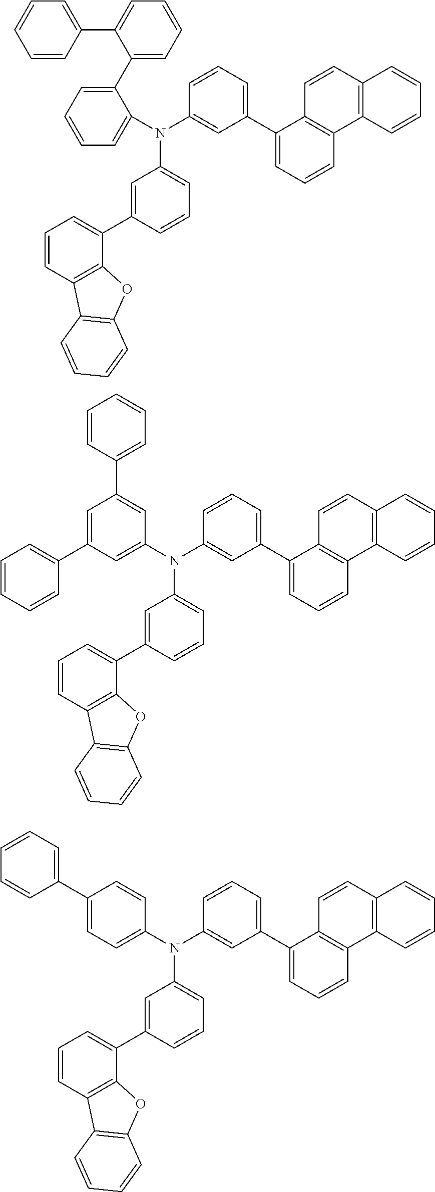

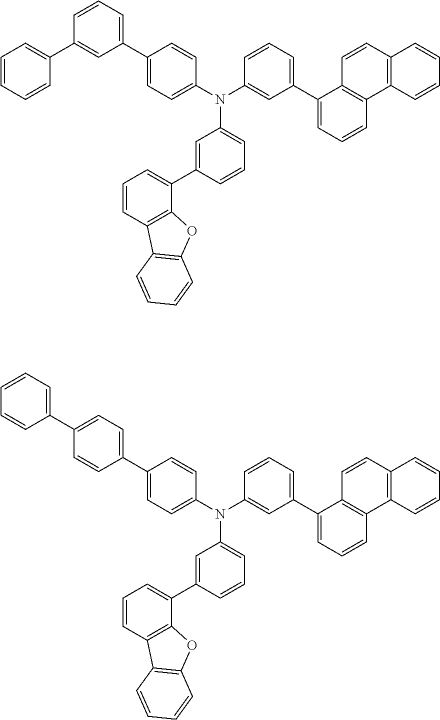 [SMILES: c1ccc(-c2cccc(-c3ccc(N(c4cccc(-c5cccc6c5ccc5ccccc56)c4)c4cccc(-c5cccc6c5oc5ccccc56)c4)cc3)c2)cc1 ; c1ccc(-c2ccc(-c3ccc(N(c4cccc(-c5cccc6c5ccc5ccccc56)c4)c4cccc(-c5cccc6c5oc5ccccc56)c4)cc3)cc2)cc1]

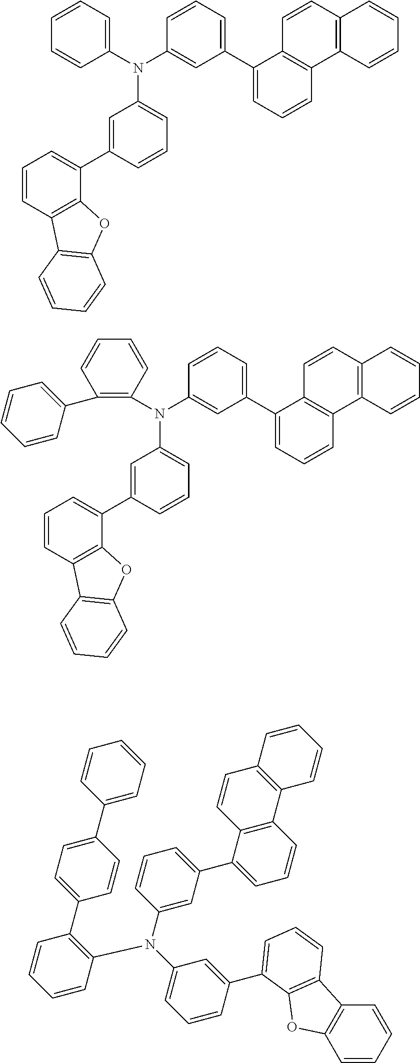

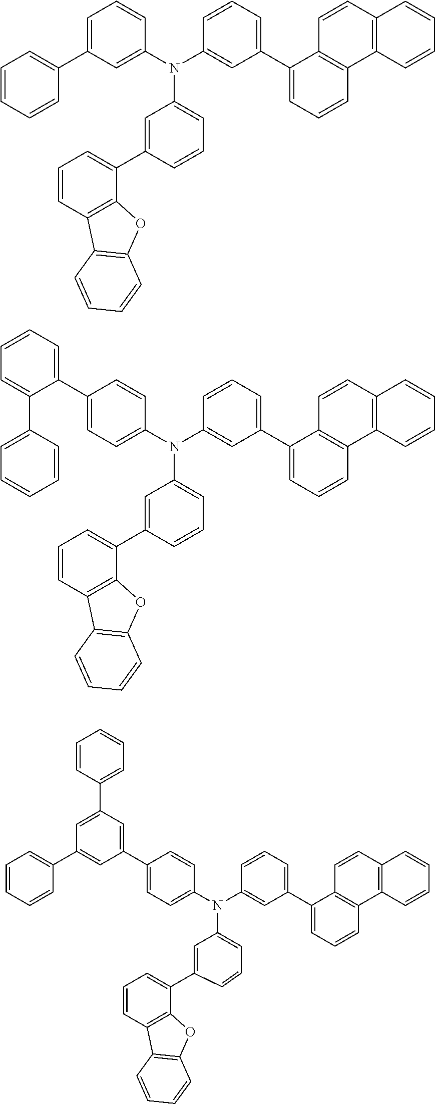

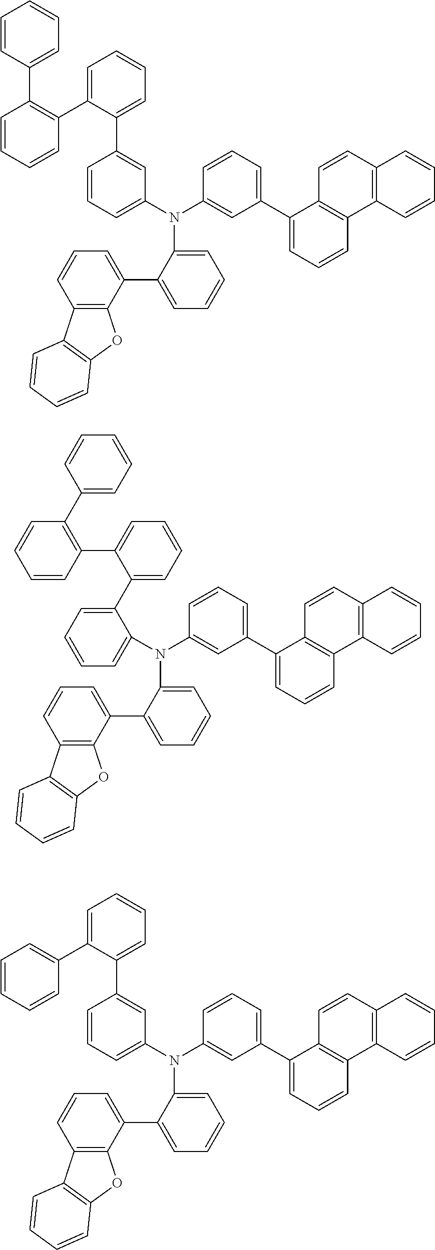

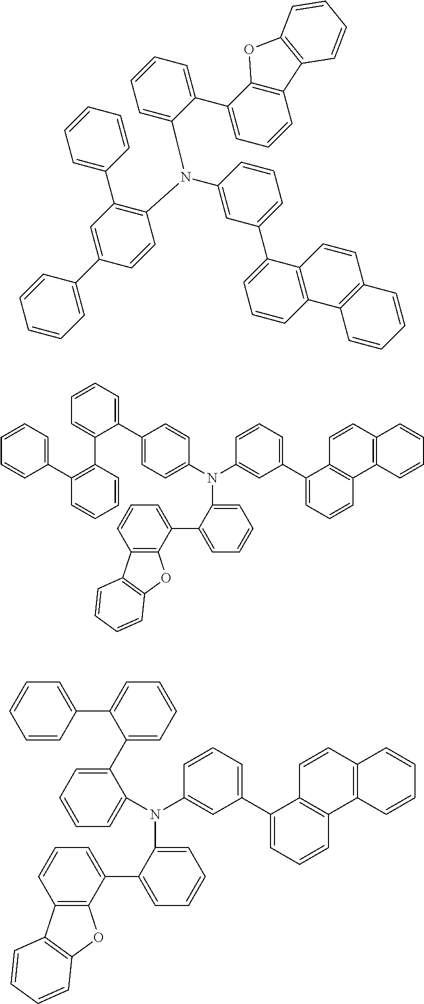 [SMILES: c1ccc(-c2ccc(N(c3cccc(-c4cccc5c4ccc4ccccc45)c3)c3ccccc3-c3cccc4c3oc3ccccc34)c(-c3ccccc3)c2)cc1 ; c1ccc(-c2ccccc2-c2ccccc2-c2ccc(N(c3cccc(-c4cccc5c4ccc4ccccc45)c3)c3ccccc3-c3cccc4c3oc3ccccc34)cc2)cc1 ; c1ccc(-c2ccccc2-c2ccccc2N(c2cccc(-c3cccc4c3ccc3ccccc34)c2)c2ccccc2-c2cccc3c2oc2ccccc23)cc1]

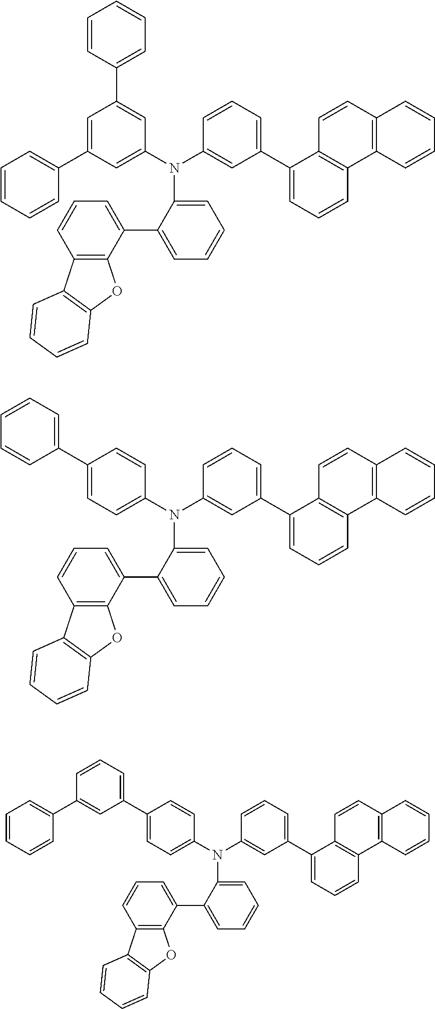 [SMILES: c1ccc(-c2cc(-c3ccccc3)cc(N(c3cccc(-c4cccc5c4ccc4ccccc45)c3)c3ccccc3-c3cccc4c3oc3ccccc34)c2)cc1 ; c1ccc(-c2ccc(N(c3cccc(-c4cccc5c4ccc4ccccc45)c3)c3ccccc3-c3cccc4c3oc3ccccc34)cc2)cc1 ; c1ccc(-c2cccc(-c3ccc(N(c4cccc(-c5cccc6c5ccc5ccccc56)c4)c4ccccc4-c4cccc5c4oc4ccccc45)cc3)c2)cc1]

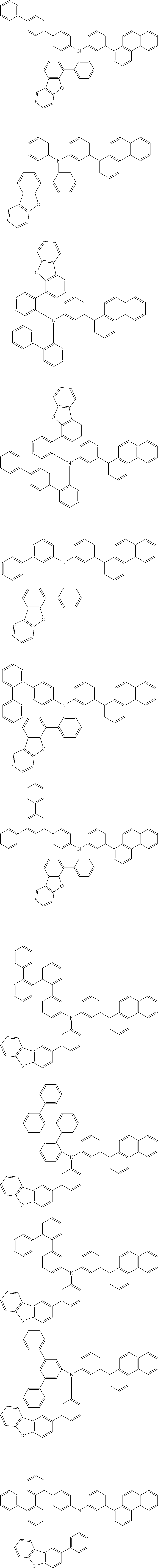

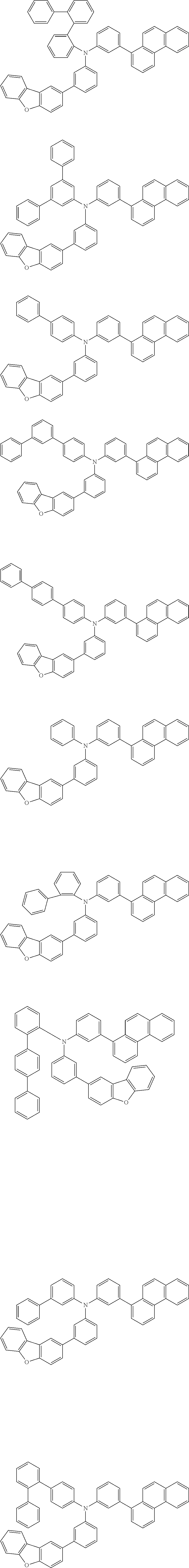

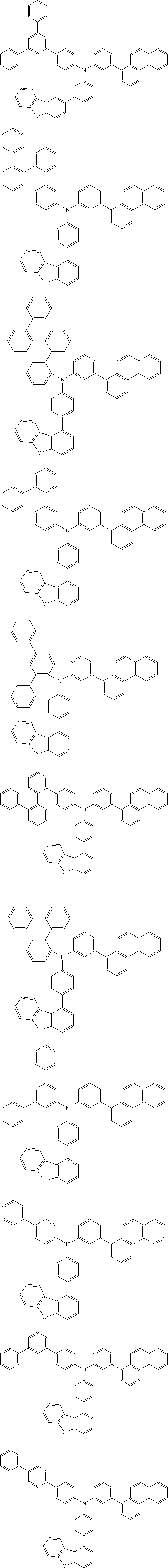

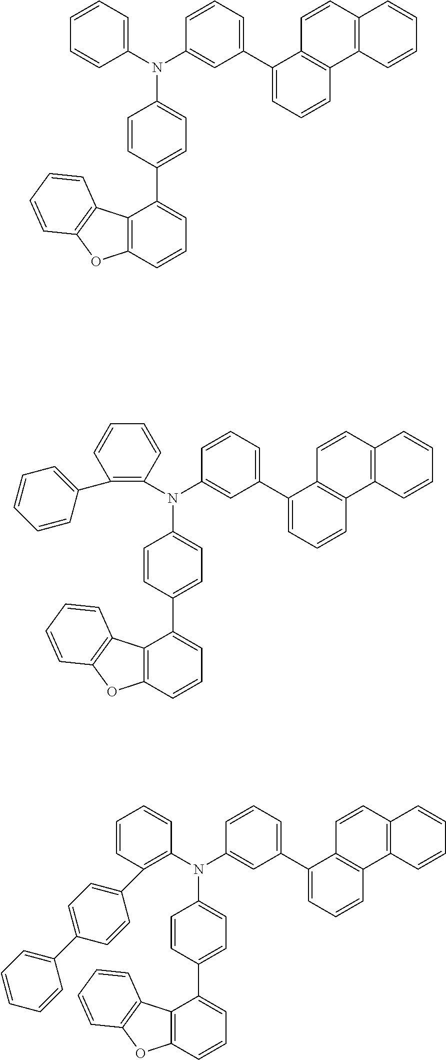 [SMILES: c1ccc(N(c2ccc(-c3cccc4oc5ccccc5c34)cc2)c2cccc(-c3cccc4c3ccc3ccccc34)c2)cc1 ; c1ccc(-c2ccccc2N(c2ccc(-c3cccc4oc5ccccc5c34)cc2)c2cccc(-c3cccc4c3ccc3ccccc34)c2)cc1 ; c1ccc(-c2ccc(-c3ccccc3N(c3ccc(-c4cccc5oc6ccccc6c45)cc3)c3cccc(-c4cccc5c4ccc4ccccc45)c3)cc2)cc1]

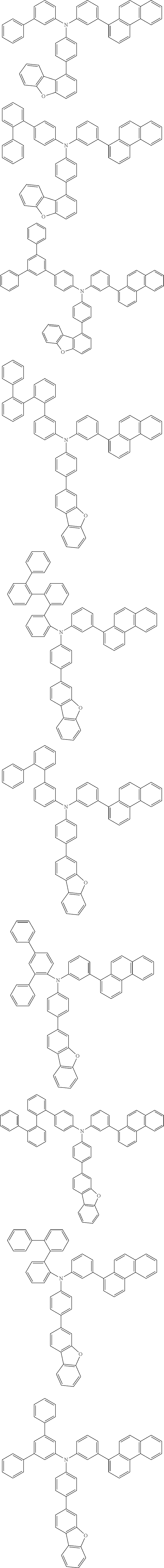

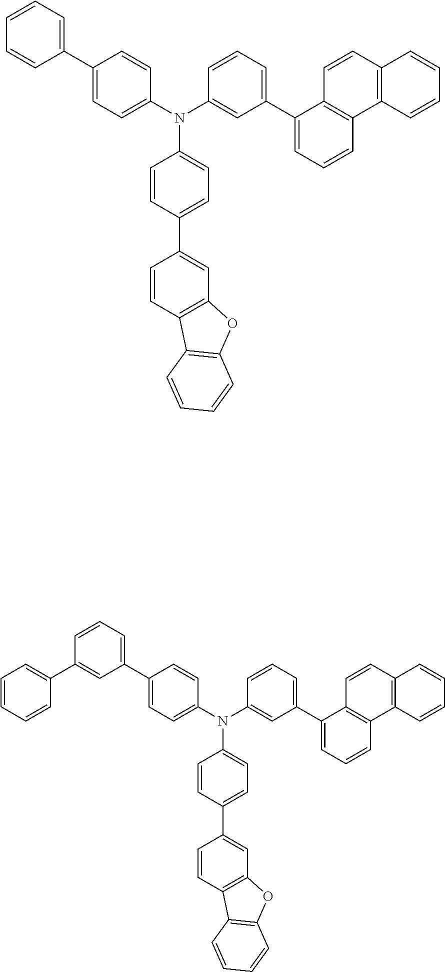 [SMILES: c1ccc(-c2ccc(N(c3ccc(-c4ccc5c(c4)oc4ccccc45)cc3)c3cccc(-c4cccc5c4ccc4ccccc45)c3)cc2)cc1 ; c1ccc(-c2cccc(-c3ccc(N(c4ccc(-c5ccc6c(c5)oc5ccccc56)cc4)c4cccc(-c5cccc6c5ccc5ccccc56)c4)cc3)c2)cc1]

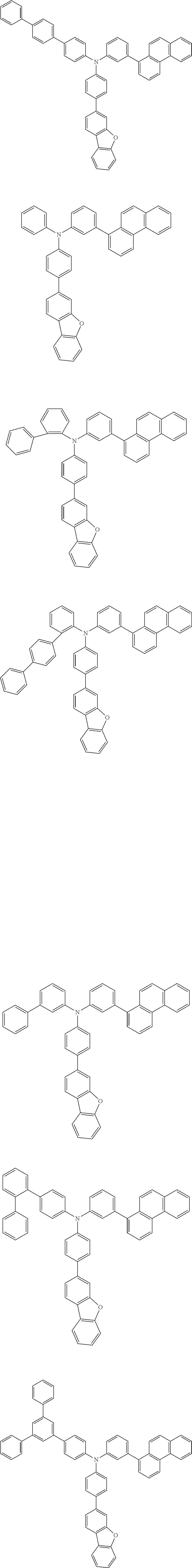

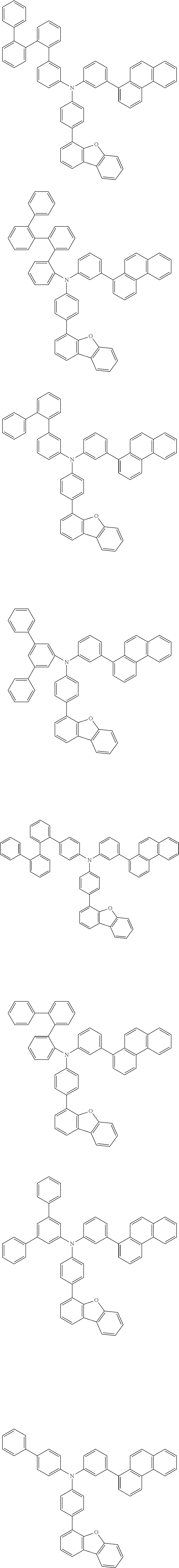

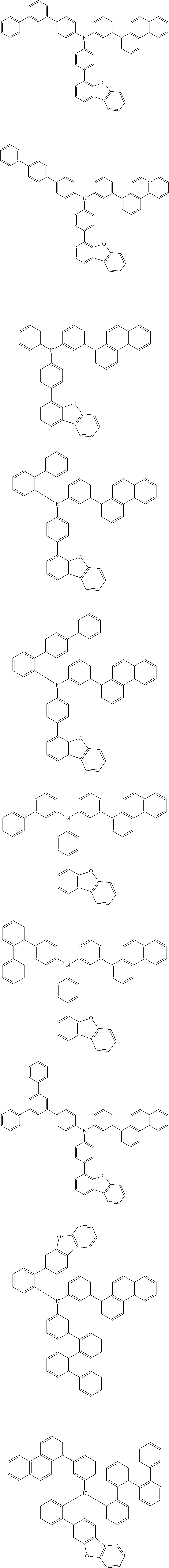

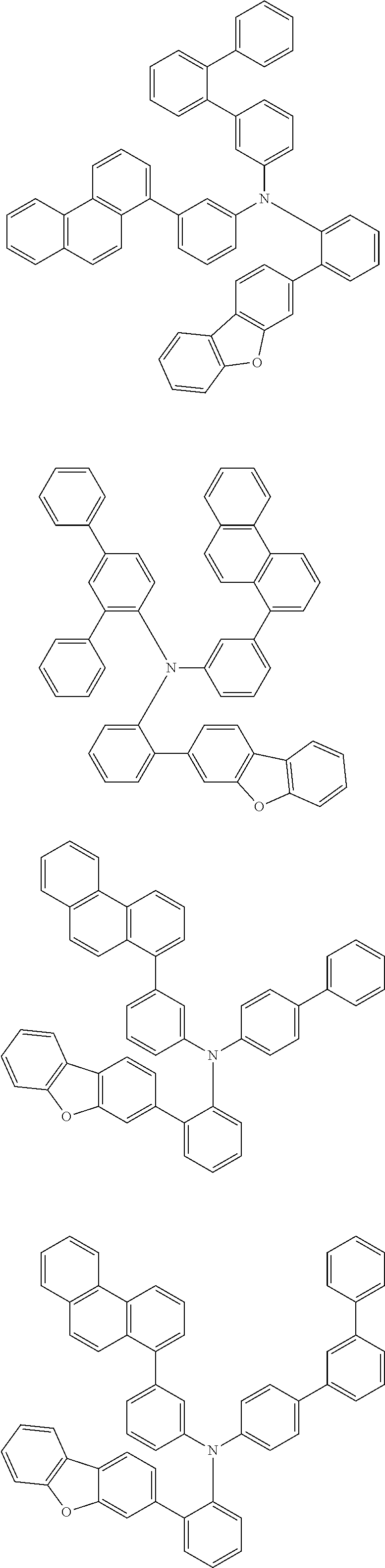 [SMILES: c1ccc(-c2ccccc2-c2cccc(N(c3cccc(-c4cccc5c4ccc4ccccc45)c3)c3ccccc3-c3ccc4c(c3)oc3ccccc34)c2)cc1 ; c1ccc(-c2ccc(N(c3cccc(-c4cccc5c4ccc4ccccc45)c3)c3ccccc3-c3ccc4c(c3)oc3ccccc34)c(-c3ccccc3)c2)cc1 ; c1ccc(-c2ccc(N(c3cccc(-c4cccc5c4ccc4ccccc45)c3)c3ccccc3-c3ccc4c(c3)oc3ccccc34)cc2)cc1 ; c1ccc(-c2cccc(-c3ccc(N(c4cccc(-c5cccc6c5ccc5ccccc56)c4)c4ccccc4-c4ccc5c(c4)oc4ccccc45)cc3)c2)cc1]

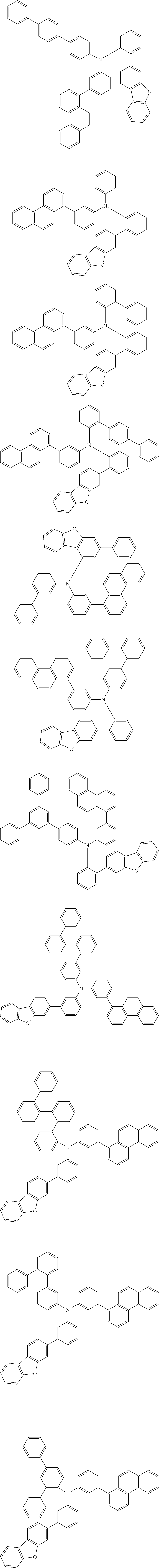

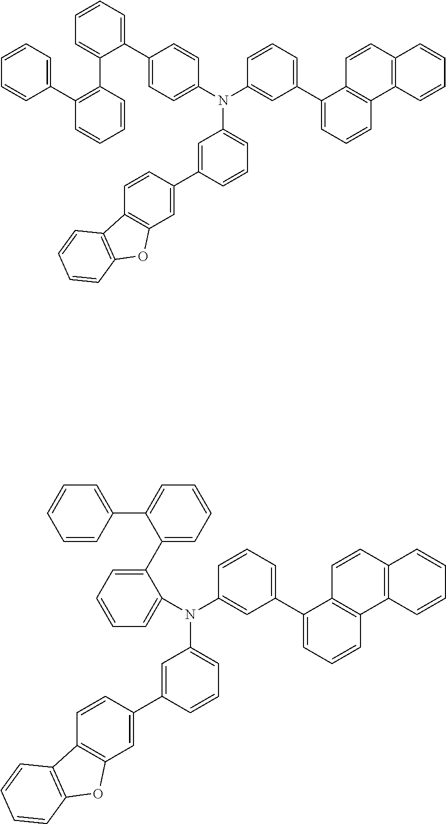 [SMILES: c1ccc(-c2ccccc2-c2ccccc2-c2ccc(N(c3cccc(-c4ccc5c(c4)oc4ccccc45)c3)c3cccc(-c4cccc5c4ccc4ccccc45)c3)cc2)cc1 ; c1ccc(-c2ccccc2-c2ccccc2N(c2cccc(-c3ccc4c(c3)oc3ccccc34)c2)c2cccc(-c3cccc4c3ccc3ccccc34)c2)cc1]

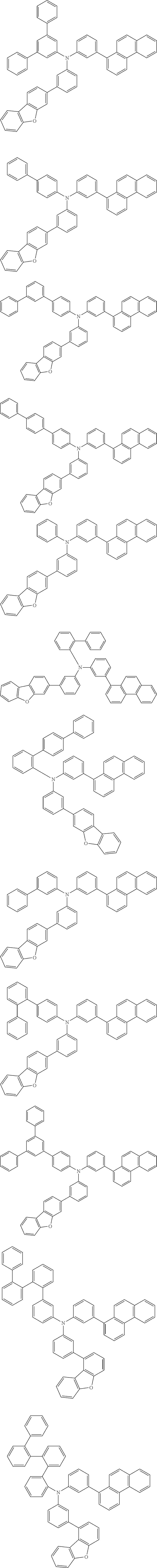

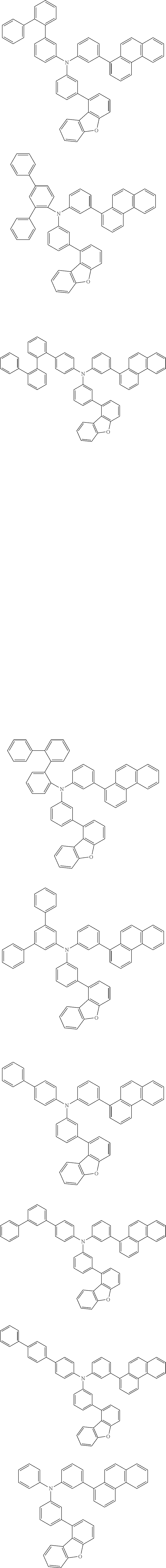

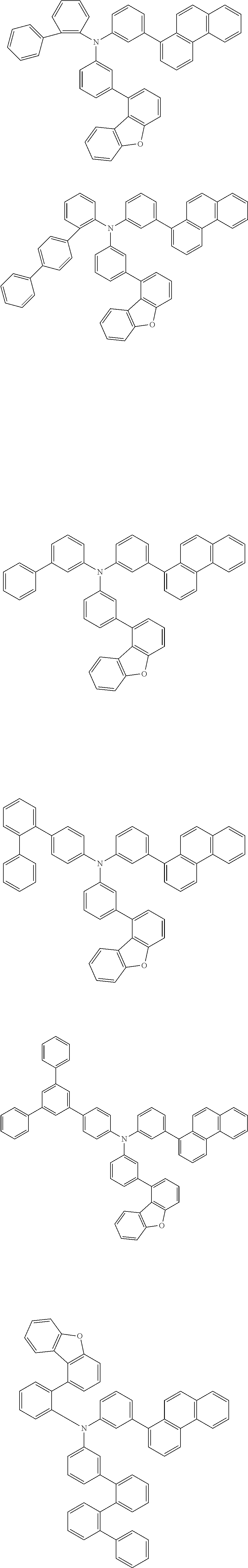

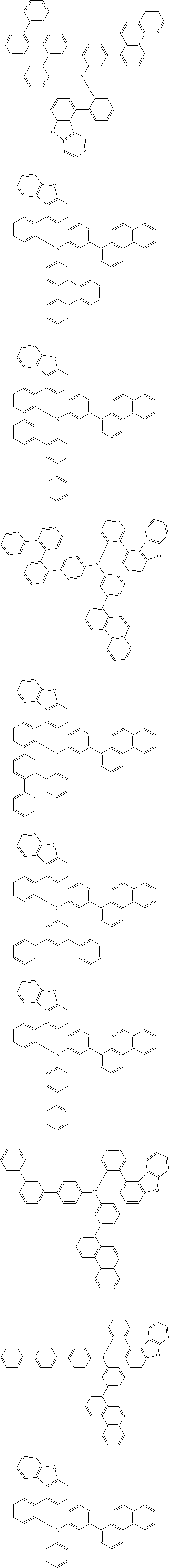

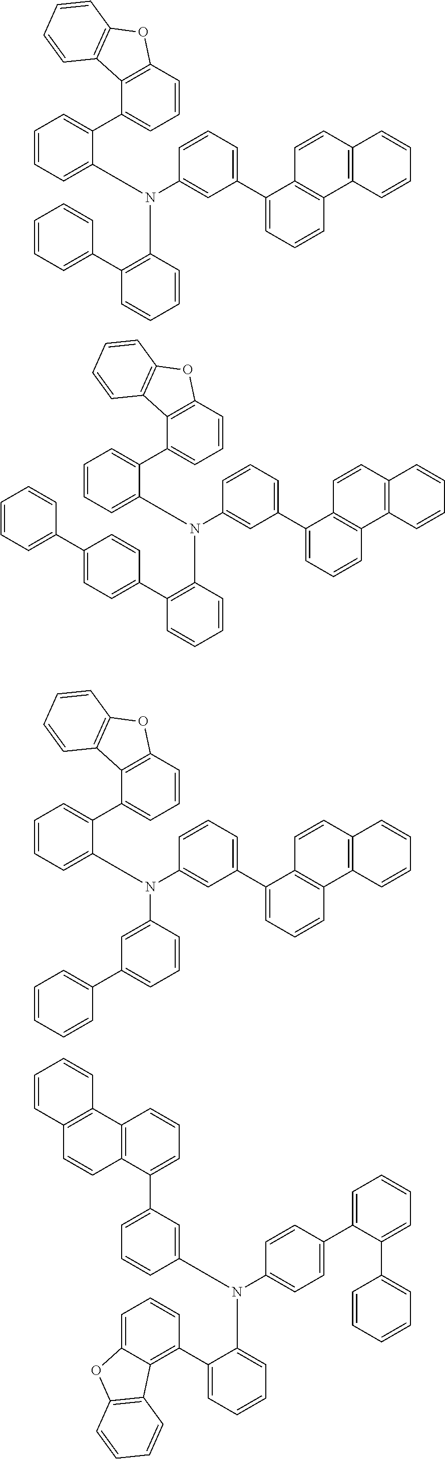

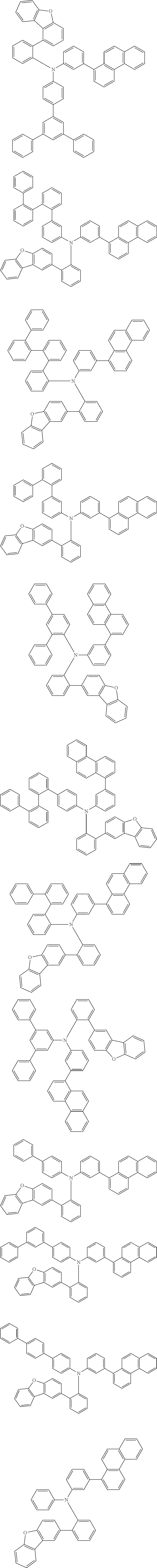

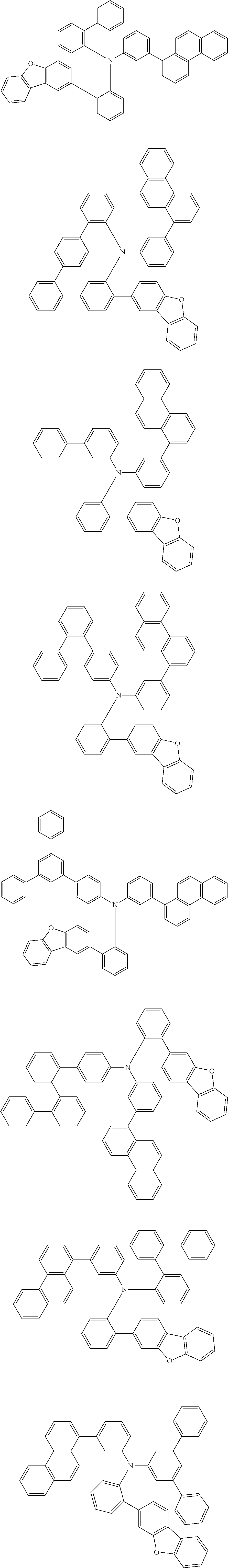

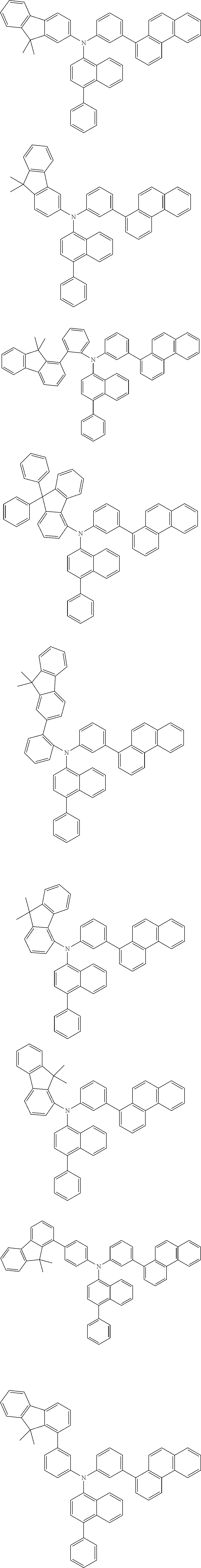

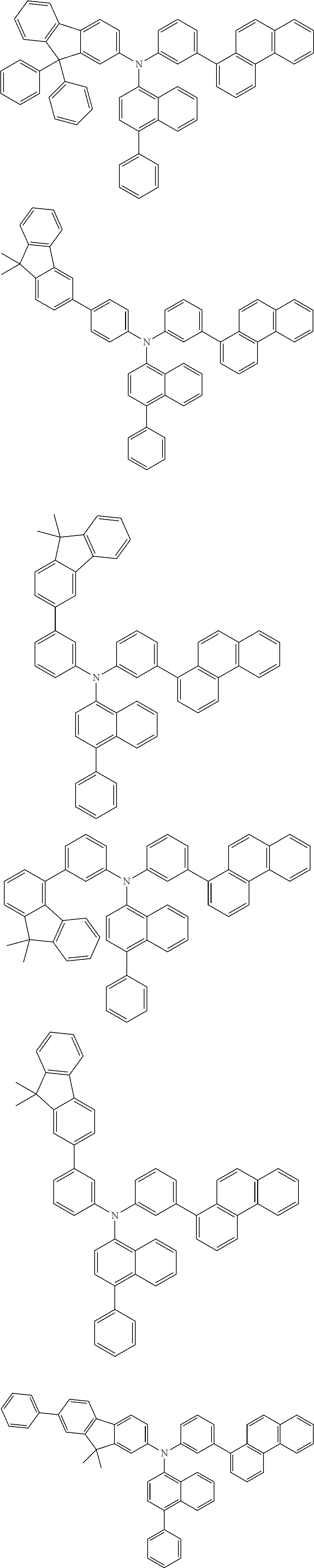

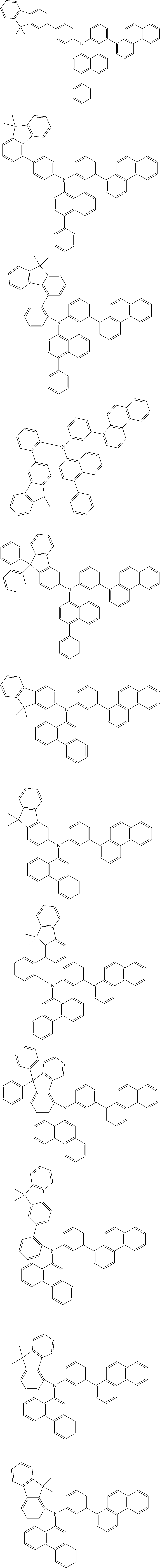

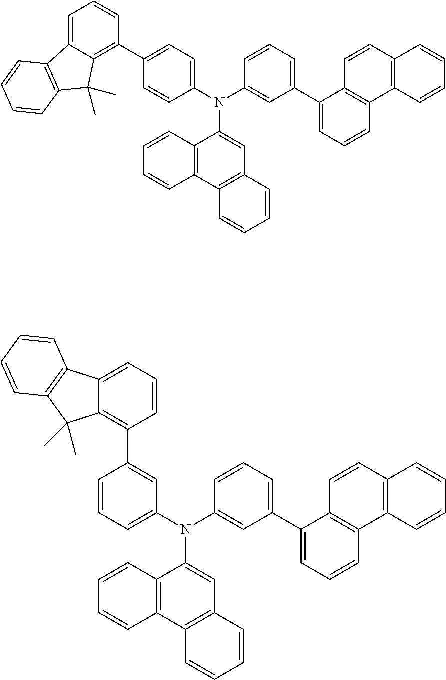 [SMILES: CC1(C)c2ccccc2-c2cccc(-c3ccc(N(c4cccc(-c5cccc6c5ccc5ccccc56)c4)c4cc5ccccc5c5ccccc45)cc3)c21 ; CC1(C)c2ccccc2-c2cccc(-c3cccc(N(c4cccc(-c5cccc6c5ccc5ccccc56)c4)c4cc5ccccc5c5ccccc45)c3)c21]

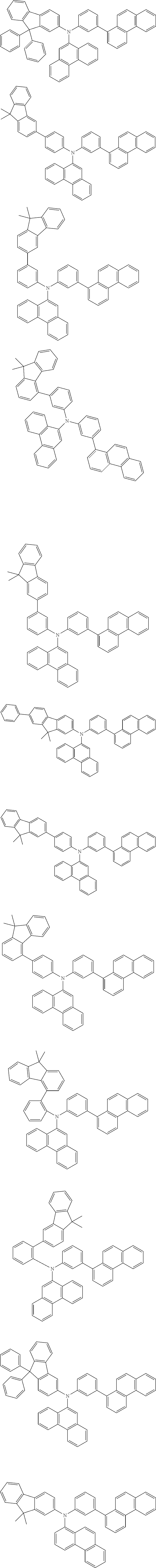

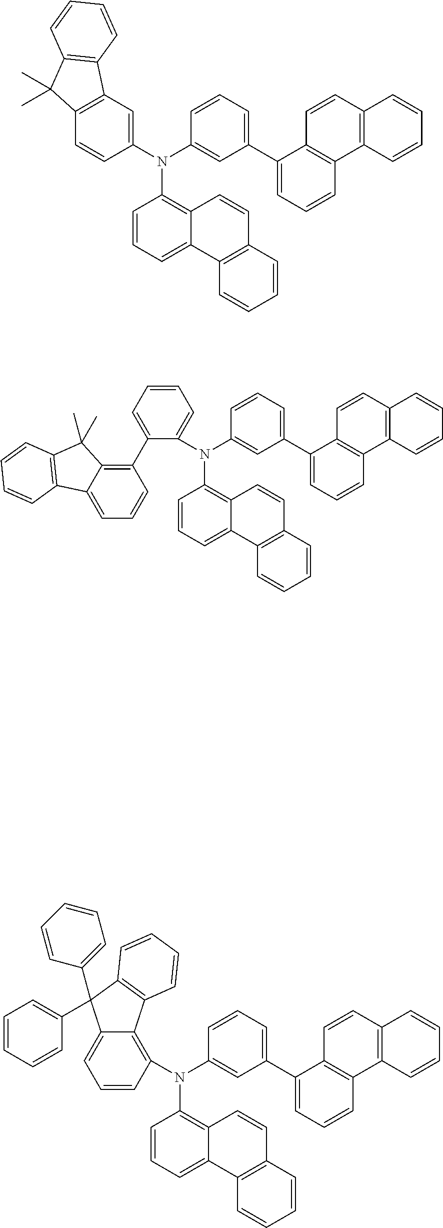 [SMILES: CC1(C)c2ccccc2-c2cc(N(c3cccc(-c4cccc5c4ccc4ccccc45)c3)c3cccc4c3ccc3ccccc34)ccc21 ; CC1(C)c2ccccc2-c2cccc(-c3ccccc3N(c3cccc(-c4cccc5c4ccc4ccccc45)c3)c3cccc4c3ccc3ccccc34)c21 ; c1ccc(C2(c3ccccc3)c3ccccc3-c3c(N(c4cccc(-c5cccc6c5ccc5ccccc56)c4)c4cccc5c4ccc4ccccc45)cccc32)cc1]

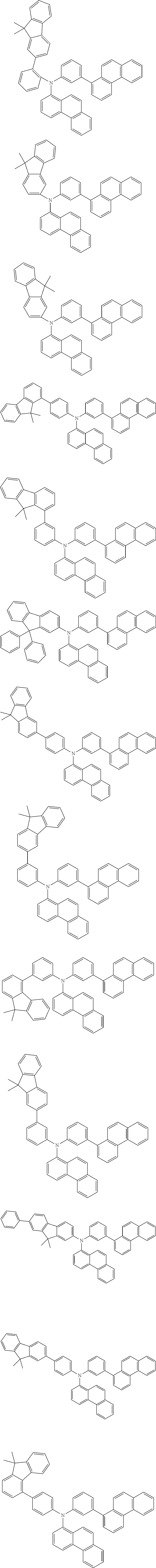

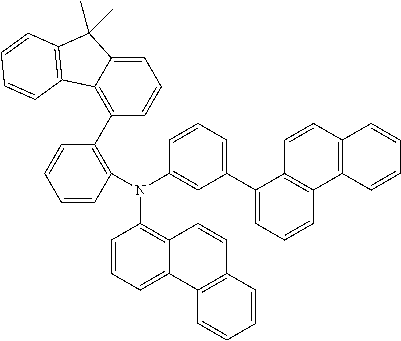 [SMILES: CC1(C)c2ccccc2-c2c(-c3ccccc3N(c3cccc(-c4cccc5c4ccc4ccccc45)c3)c3cccc4c3ccc3ccccc34)cccc21]

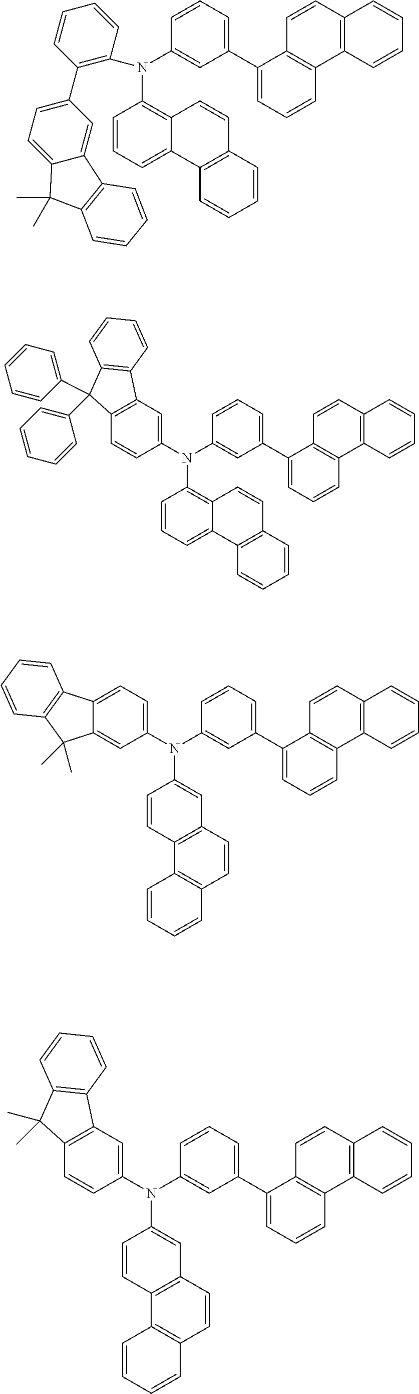 [SMILES: CC1(C)c2ccccc2-c2cc(-c3ccccc3N(c3cccc(-c4cccc5c4ccc4ccccc45)c3)c3cccc4c3ccc3ccccc34)ccc21 ; c1ccc(C2(c3ccccc3)c3ccccc3-c3cc(N(c4cccc(-c5cccc6c5ccc5ccccc56)c4)c4cccc5c4ccc4ccccc45)ccc32)cc1 ; CC1(C)c2ccccc2-c2ccc(N(c3cccc(-c4cccc5c4ccc4ccccc45)c3)c3ccc4c(ccc5ccccc54)c3)cc21 ; CC1(C)c2ccccc2-c2cc(N(c3cccc(-c4cccc5c4ccc4ccccc45)c3)c3ccc4c(ccc5ccccc54)c3)ccc21]

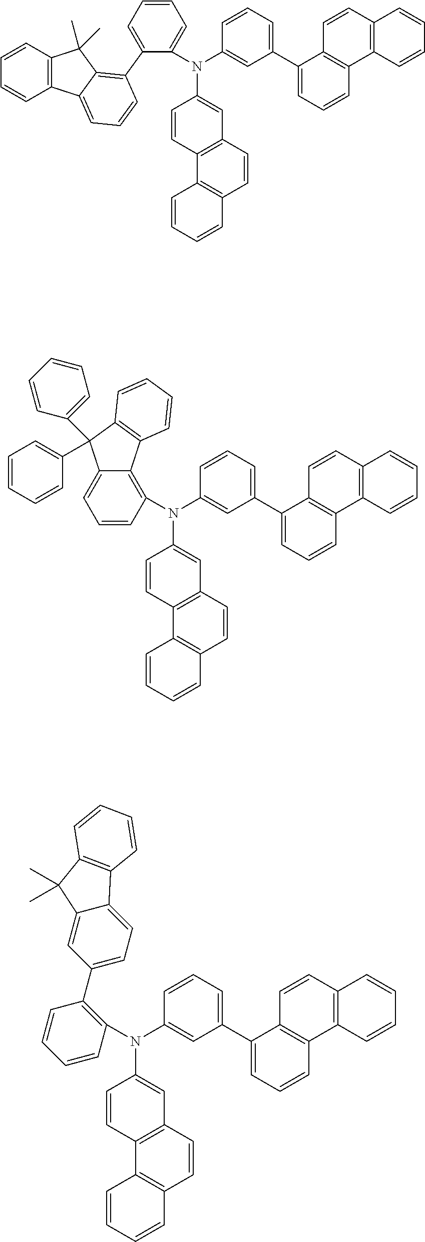 [SMILES: CC1(C)c2ccccc2-c2cccc(-c3ccccc3N(c3cccc(-c4cccc5c4ccc4ccccc45)c3)c3ccc4c(ccc5ccccc54)c3)c21 ; c1ccc(C2(c3ccccc3)c3ccccc3-c3c(N(c4cccc(-c5cccc6c5ccc5ccccc56)c4)c4ccc5c(ccc6ccccc65)c4)cccc32)cc1 ; CC1(C)c2ccccc2-c2ccc(-c3ccccc3N(c3cccc(-c4cccc5c4ccc4ccccc45)c3)c3ccc4c(ccc5ccccc54)c3)cc21]

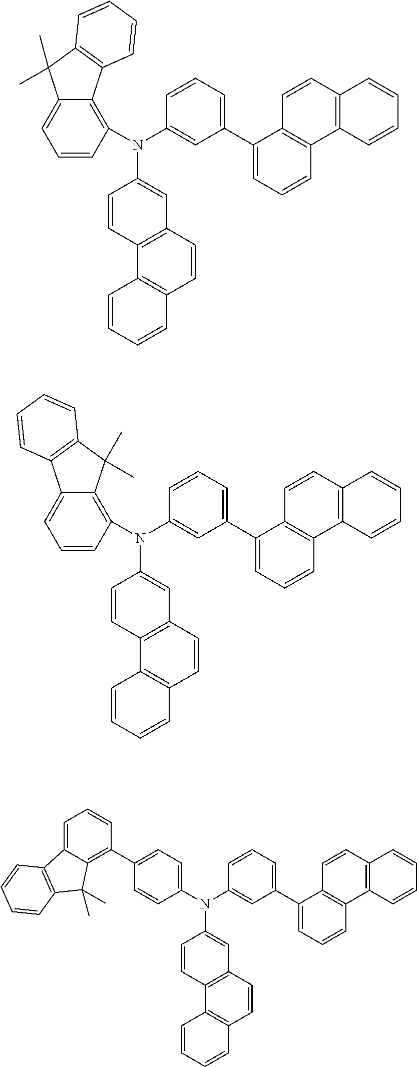 [SMILES: CC1(C)c2ccccc2-c2c(N(c3cccc(-c4cccc5c4ccc4ccccc45)c3)c3ccc4c(ccc5ccccc54)c3)cccc21 ; CC1(C)c2ccccc2-c2cccc(N(c3cccc(-c4cccc5c4ccc4ccccc45)c3)c3ccc4c(ccc5ccccc54)c3)c21 ; CC1(C)c2ccccc2-c2cccc(-c3ccc(N(c4cccc(-c5cccc6c5ccc5ccccc56)c4)c4ccc5c(ccc6ccccc65)c4)cc3)c21]

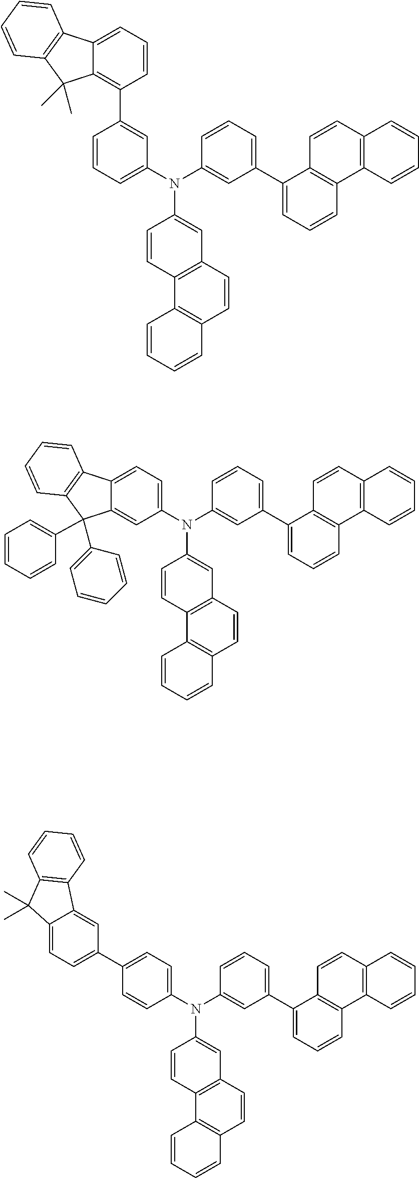 [SMILES: CC1(C)c2ccccc2-c2cccc(-c3cccc(N(c4cccc(-c5cccc6c5ccc5ccccc56)c4)c4ccc5c(ccc6ccccc65)c4)c3)c21 ; c1ccc(C2(c3ccccc3)c3ccccc3-c3ccc(N(c4cccc(-c5cccc6c5ccc5ccccc56)c4)c4ccc5c(ccc6ccccc65)c4)cc32)cc1 ; CC1(C)c2ccccc2-c2cc(-c3ccc(N(c4cccc(-c5cccc6c5ccc5ccccc56)c4)c4ccc5c(ccc6ccccc65)c4)cc3)ccc21]

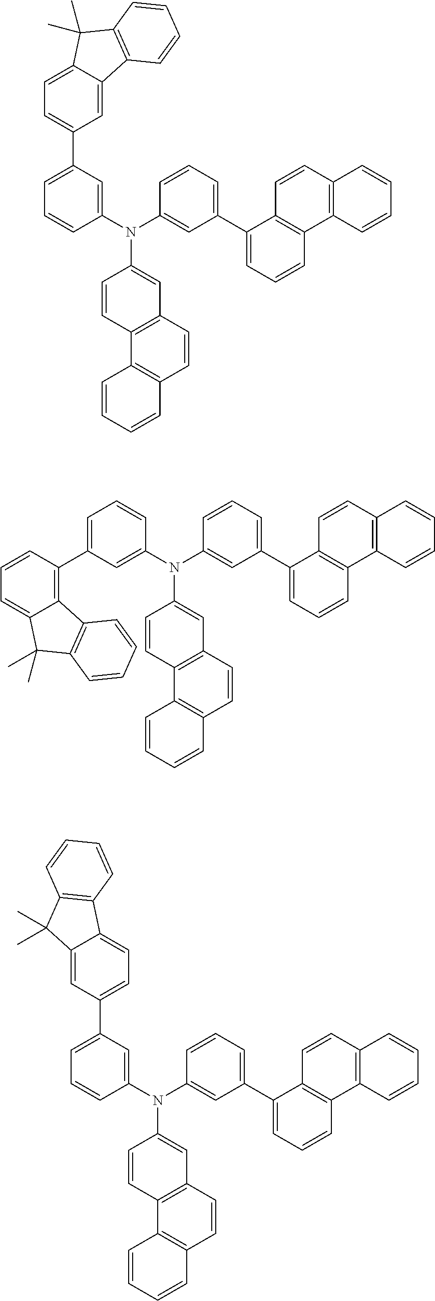 [SMILES: CC1(C)c2ccccc2-c2cc(-c3cccc(N(c4cccc(-c5cccc6c5ccc5ccccc56)c4)c4ccc5c(ccc6ccccc65)c4)c3)ccc21 ; CC1(C)c2ccccc2-c2c(-c3cccc(N(c4cccc(-c5cccc6c5ccc5ccccc56)c4)c4ccc5c(ccc6ccccc65)c4)c3)cccc21 ; CC1(C)c2ccccc2-c2ccc(-c3cccc(N(c4cccc(-c5cccc6c5ccc5ccccc56)c4)c4ccc5c(ccc6ccccc65)c4)c3)cc21]

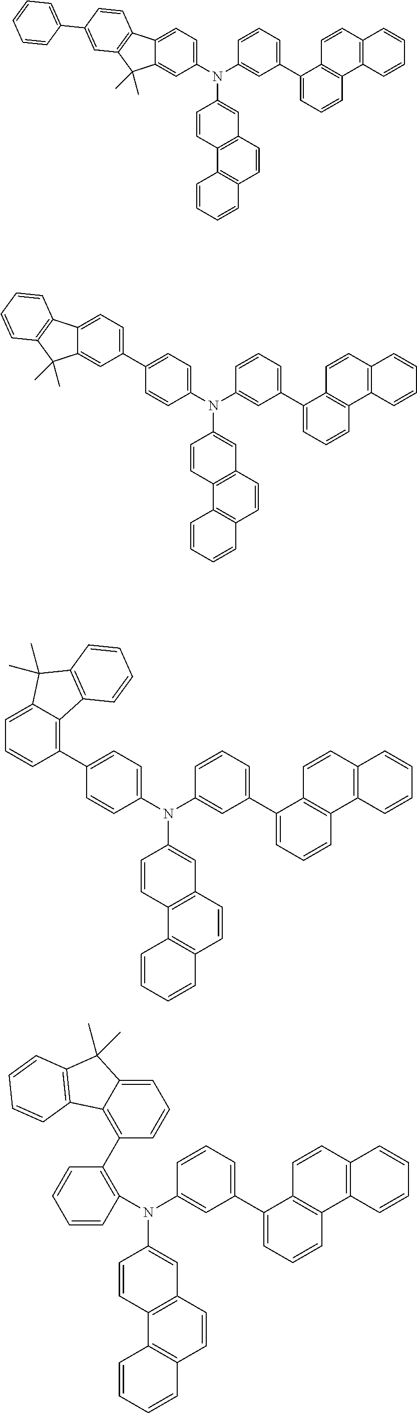 [SMILES: CC1(C)c2cc(-c3ccccc3)ccc2-c2ccc(N(c3cccc(-c4cccc5c4ccc4ccccc45)c3)c3ccc4c(ccc5ccccc54)c3)cc21 ; CC1(C)c2ccccc2-c2ccc(-c3ccc(N(c4cccc(-c5cccc6c5ccc5ccccc56)c4)c4ccc5c(ccc6ccccc65)c4)cc3)cc21 ; CC1(C)c2ccccc2-c2c(-c3ccc(N(c4cccc(-c5cccc6c5ccc5ccccc56)c4)c4ccc5c(ccc6ccccc65)c4)cc3)cccc21 ; CC1(C)c2ccccc2-c2c(-c3ccccc3N(c3cccc(-c4cccc5c4ccc4ccccc45)c3)c3ccc4c(ccc5ccccc54)c3)cccc21]

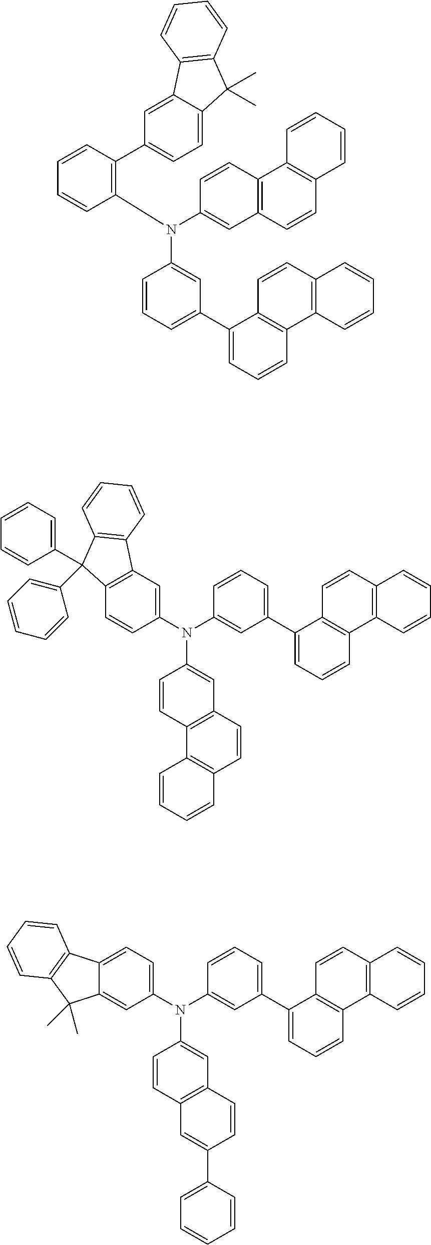 [SMILES: CC1(C)c2ccccc2-c2cc(-c3ccccc3N(c3cccc(-c4cccc5c4ccc4ccccc45)c3)c3ccc4c(ccc5ccccc54)c3)ccc21 ; c1ccc(C2(c3ccccc3)c3ccccc3-c3cc(N(c4cccc(-c5cccc6c5ccc5ccccc56)c4)c4ccc5c(ccc6ccccc65)c4)ccc32)cc1 ; CC1(C)c2ccccc2-c2ccc(N(c3cccc(-c4cccc5c4ccc4ccccc45)c3)c3ccc4cc(-c5ccccc5)ccc4c3)cc21]

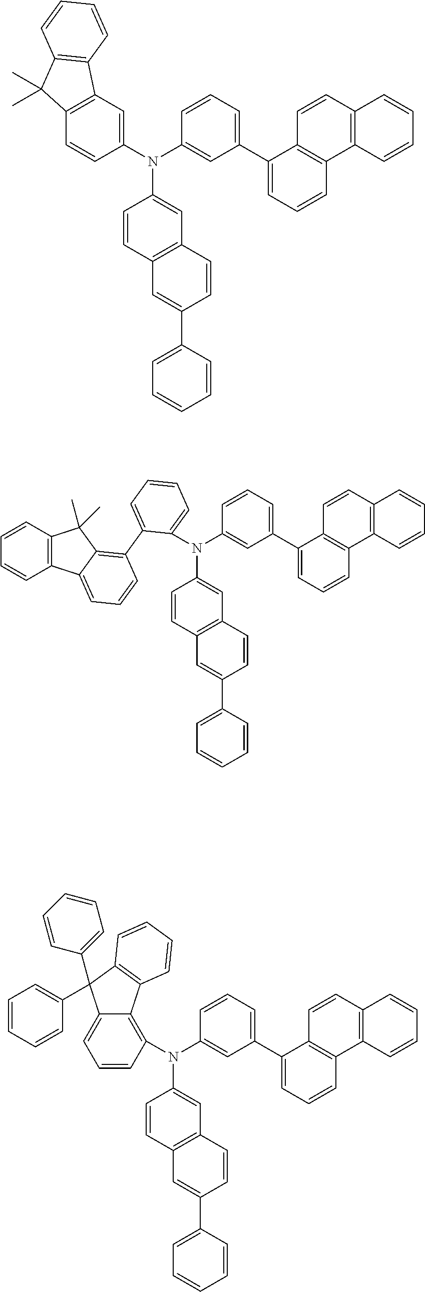 [SMILES: CC1(C)c2ccccc2-c2cc(N(c3cccc(-c4cccc5c4ccc4ccccc45)c3)c3ccc4cc(-c5ccccc5)ccc4c3)ccc21 ; CC1(C)c2ccccc2-c2cccc(-c3ccccc3N(c3cccc(-c4cccc5c4ccc4ccccc45)c3)c3ccc4cc(-c5ccccc5)ccc4c3)c21 ; c1ccc(-c2ccc3cc(N(c4cccc(-c5cccc6c5ccc5ccccc56)c4)c4cccc5c4-c4ccccc4C5(c4ccccc4)c4ccccc4)ccc3c2)cc1]

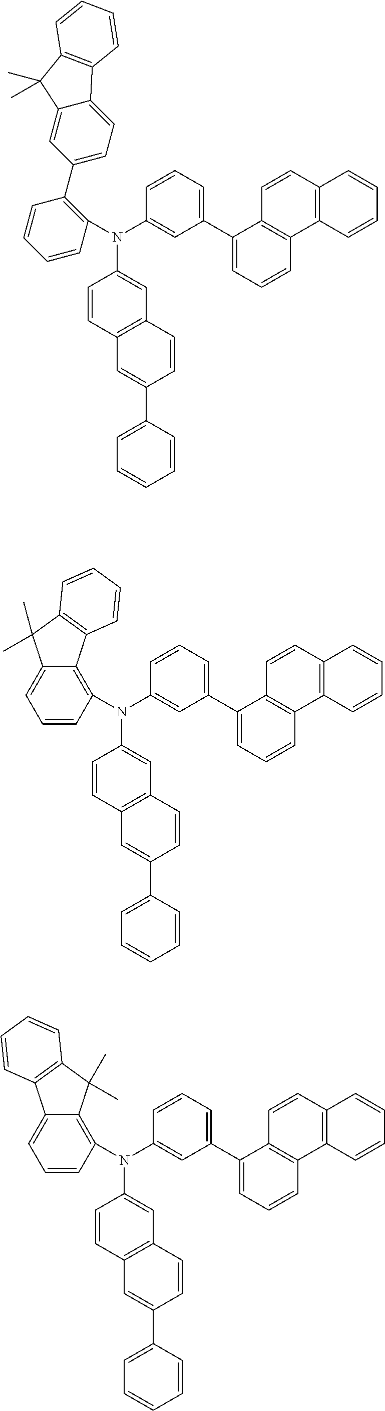 [SMILES: CC1(C)c2ccccc2-c2ccc(-c3ccccc3N(c3cccc(-c4cccc5c4ccc4ccccc45)c3)c3ccc4cc(-c5ccccc5)ccc4c3)cc21 ; CC1(C)c2ccccc2-c2c(N(c3cccc(-c4cccc5c4ccc4ccccc45)c3)c3ccc4cc(-c5ccccc5)ccc4c3)cccc21 ; CC1(C)c2ccccc2-c2cccc(N(c3cccc(-c4cccc5c4ccc4ccccc45)c3)c3ccc4cc(-c5ccccc5)ccc4c3)c21]

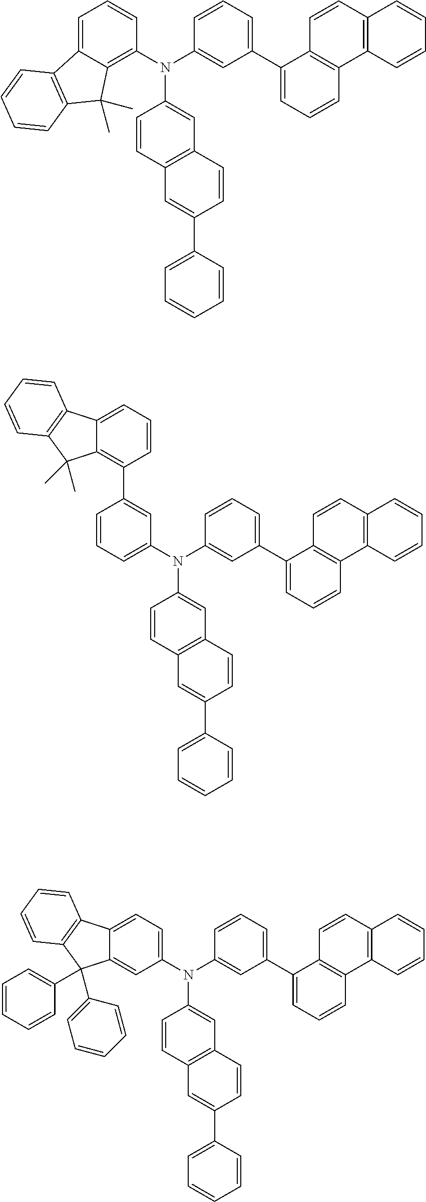 [SMILES: CC1(C)c2ccccc2-c2cccc(N(c3cccc(-c4cccc5c4ccc4ccccc45)c3)c3ccc4cc(-c5ccccc5)ccc4c3)c21 ; CC1(C)c2ccccc2-c2cccc(-c3cccc(N(c4cccc(-c5cccc6c5ccc5ccccc56)c4)c4ccc5cc(-c6ccccc6)ccc5c4)c3)c21 ; c1ccc(-c2ccc3cc(N(c4cccc(-c5cccc6c5ccc5ccccc56)c4)c4ccc5c(c4)C(c4ccccc4)(c4ccccc4)c4ccccc4-5)ccc3c2)cc1]

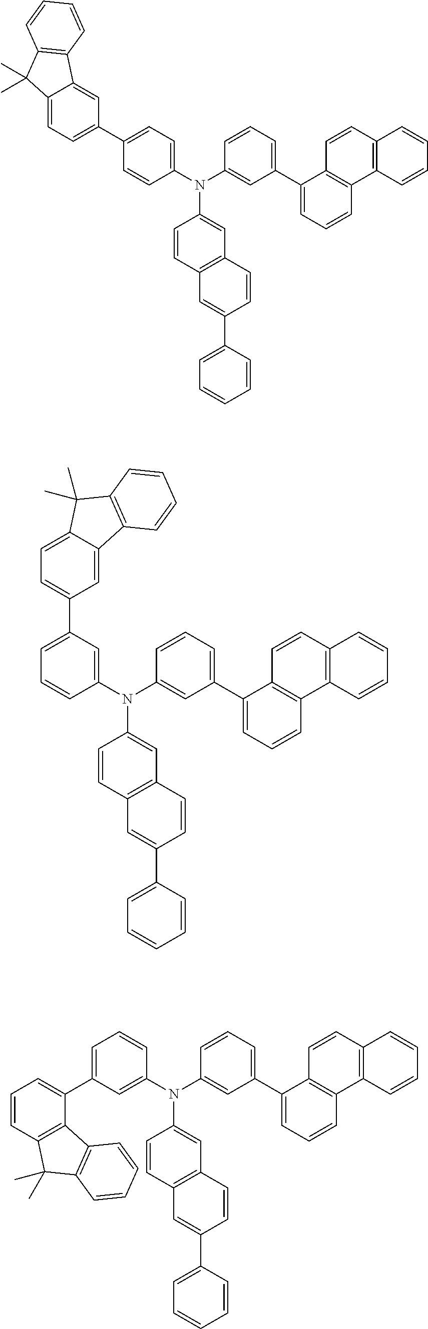 [SMILES: CC1(C)c2ccccc2-c2cc(-c3ccc(N(c4cccc(-c5cccc6c5ccc5ccccc56)c4)c4ccc5cc(-c6ccccc6)ccc5c4)cc3)ccc21 ; CC1(C)c2ccccc2-c2cc(-c3cccc(N(c4cccc(-c5cccc6c5ccc5ccccc56)c4)c4ccc5cc(-c6ccccc6)ccc5c4)c3)ccc21 ; CC1(C)c2ccccc2-c2c(-c3cccc(N(c4cccc(-c5cccc6c5ccc5ccccc56)c4)c4ccc5cc(-c6ccccc6)ccc5c4)c3)cccc21]

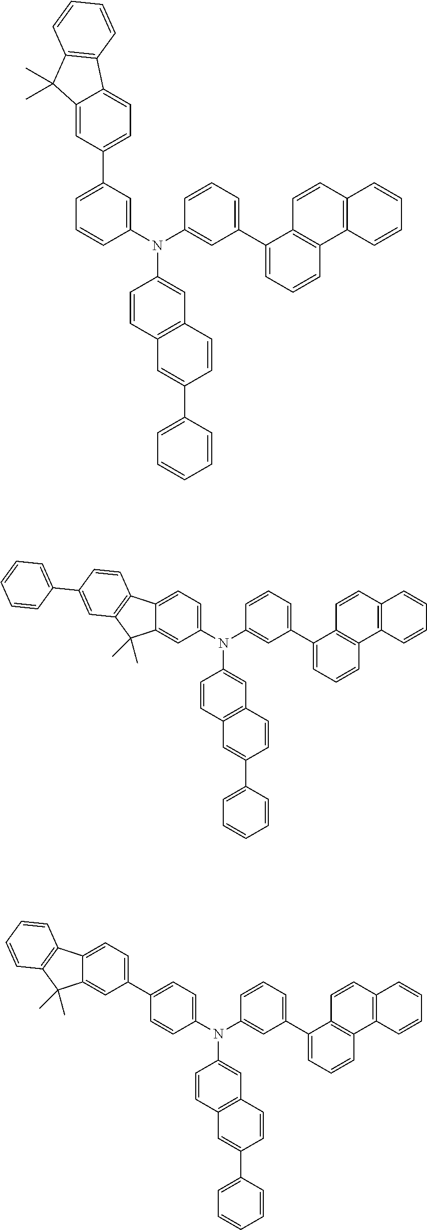 [SMILES: CC1(C)c2ccccc2-c2ccc(-c3cccc(N(c4cccc(-c5cccc6c5ccc5ccccc56)c4)c4ccc5cc(-c6ccccc6)ccc5c4)c3)cc21 ; CC1(C)c2cc(-c3ccccc3)ccc2-c2ccc(N(c3cccc(-c4cccc5c4ccc4ccccc45)c3)c3ccc4cc(-c5ccccc5)ccc4c3)cc21 ; CC1(C)c2ccccc2-c2ccc(-c3ccc(N(c4cccc(-c5cccc6c5ccc5ccccc56)c4)c4ccc5cc(-c6ccccc6)ccc5c4)cc3)cc21]

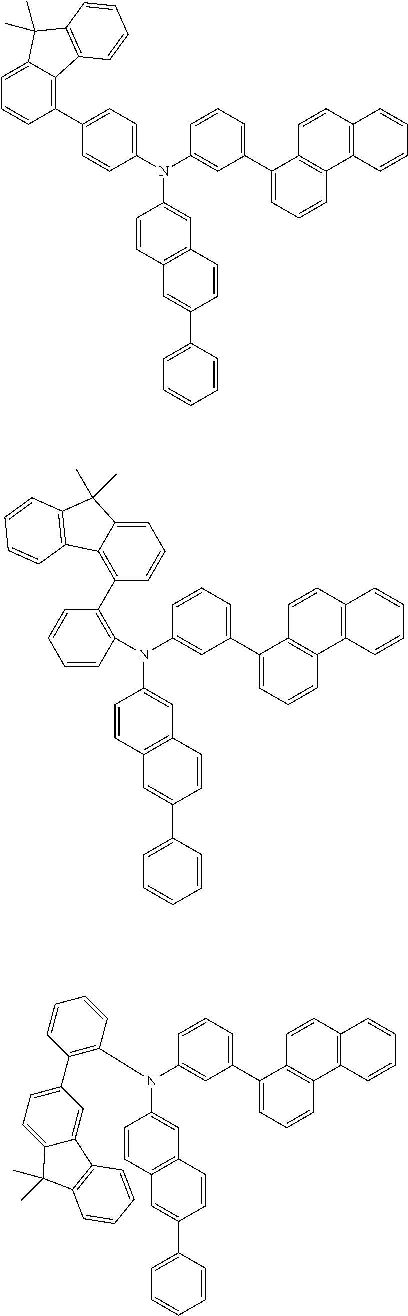 [SMILES: CC1(C)c2ccccc2-c2c(-c3ccc(N(c4cccc(-c5cccc6c5ccc5ccccc56)c4)c4ccc5cc(-c6ccccc6)ccc5c4)cc3)cccc21 ; CC1(C)c2ccccc2-c2c(-c3ccccc3N(c3cccc(-c4cccc5c4ccc4ccccc45)c3)c3ccc4cc(-c5ccccc5)ccc4c3)cccc21 ; CC1(C)c2ccccc2-c2cc(-c3ccccc3N(c3cccc(-c4cccc5c4ccc4ccccc45)c3)c3ccc4cc(-c5ccccc5)ccc4c3)ccc21]

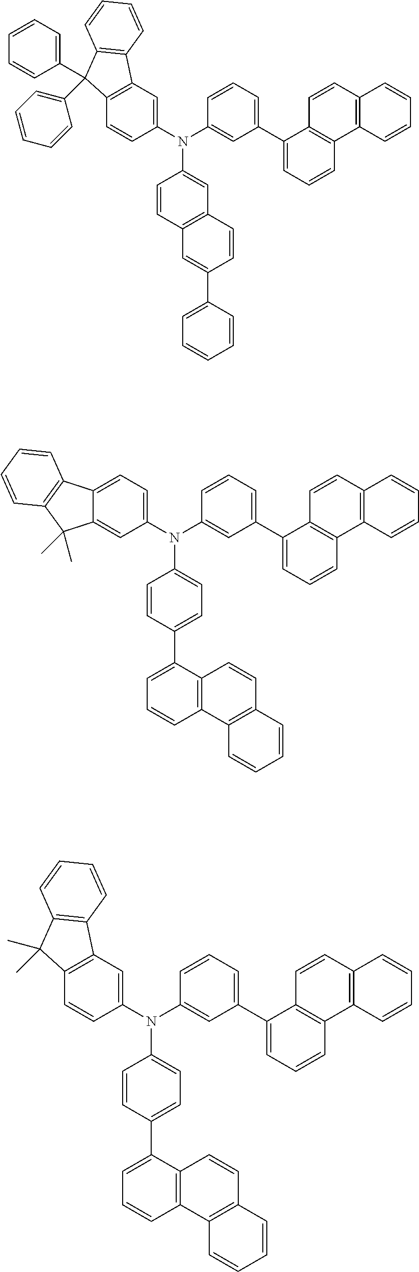 [SMILES: c1ccc(-c2ccc3cc(N(c4cccc(-c5cccc6c5ccc5ccccc56)c4)c4ccc5c(c4)-c4ccccc4C5(c4ccccc4)c4ccccc4)ccc3c2)cc1 ; CC1(C)c2ccccc2-c2ccc(N(c3ccc(-c4cccc5c4ccc4ccccc45)cc3)c3cccc(-c4cccc5c4ccc4ccccc45)c3)cc21 ; CC1(C)c2ccccc2-c2cc(N(c3ccc(-c4cccc5c4ccc4ccccc45)cc3)c3cccc(-c4cccc5c4ccc4ccccc45)c3)ccc21]

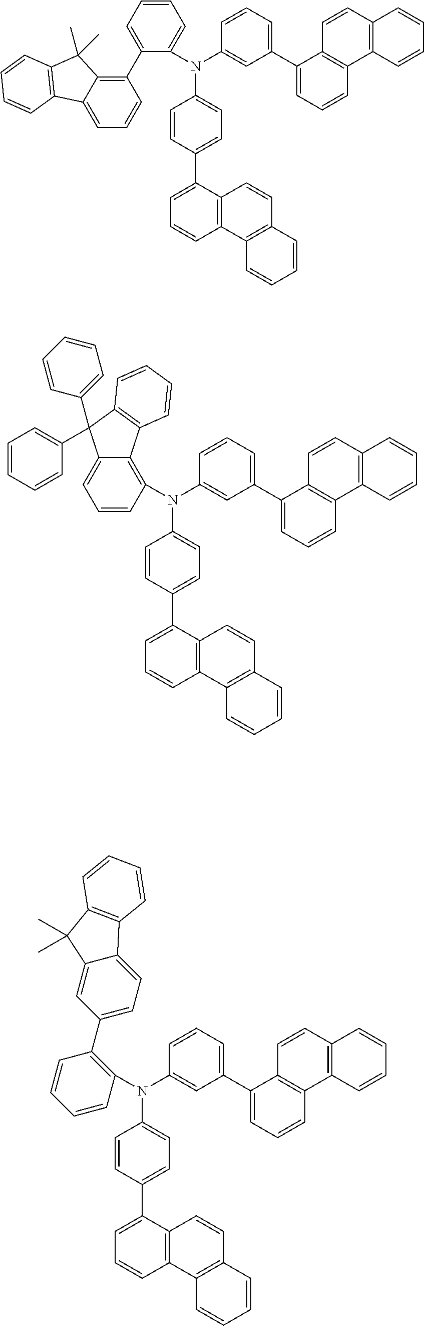 [SMILES: CC1(C)c2ccccc2-c2cccc(-c3ccccc3N(c3ccc(-c4cccc5c4ccc4ccccc45)cc3)c3cccc(-c4cccc5c4ccc4ccccc45)c3)c21 ; c1ccc(C2(c3ccccc3)c3ccccc3-c3c(N(c4ccc(-c5cccc6c5ccc5ccccc56)cc4)c4cccc(-c5cccc6c5ccc5ccccc56)c4)cccc32)cc1 ; CC1(C)c2ccccc2-c2ccc(-c3ccccc3N(c3ccc(-c4cccc5c4ccc4ccccc45)cc3)c3cccc(-c4cccc5c4ccc4ccccc45)c3)cc21]

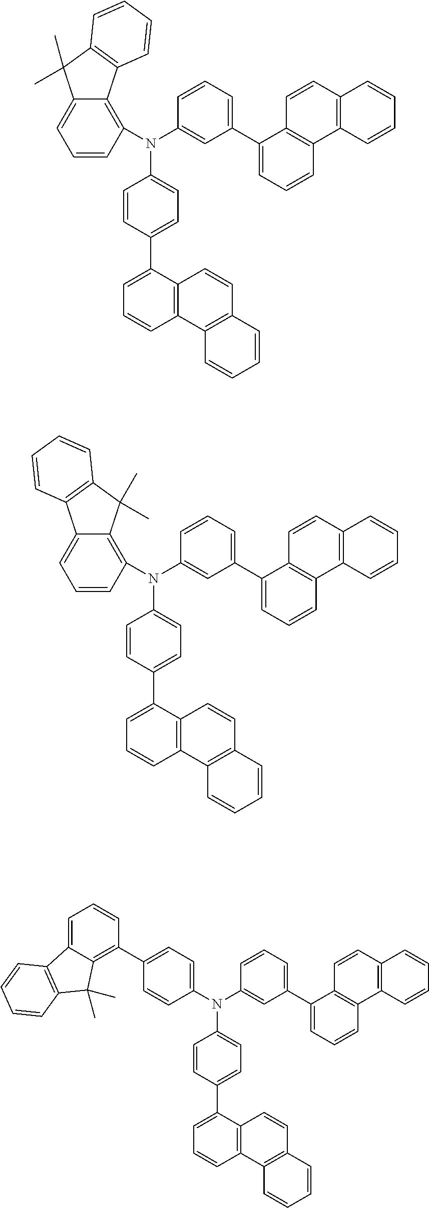 [SMILES: CC1(C)c2ccccc2-c2c(N(c3ccc(-c4cccc5c4ccc4ccccc45)cc3)c3cccc(-c4cccc5c4ccc4ccccc45)c3)cccc21 ; CC1(C)c2ccccc2-c2cccc(N(c3ccc(-c4cccc5c4ccc4ccccc45)cc3)c3cccc(-c4cccc5c4ccc4ccccc45)c3)c21 ; CC1(C)c2ccccc2-c2cccc(-c3ccc(N(c4ccc(-c5cccc6c5ccc5ccccc56)cc4)c4cccc(-c5cccc6c5ccc5ccccc56)c4)cc3)c21]

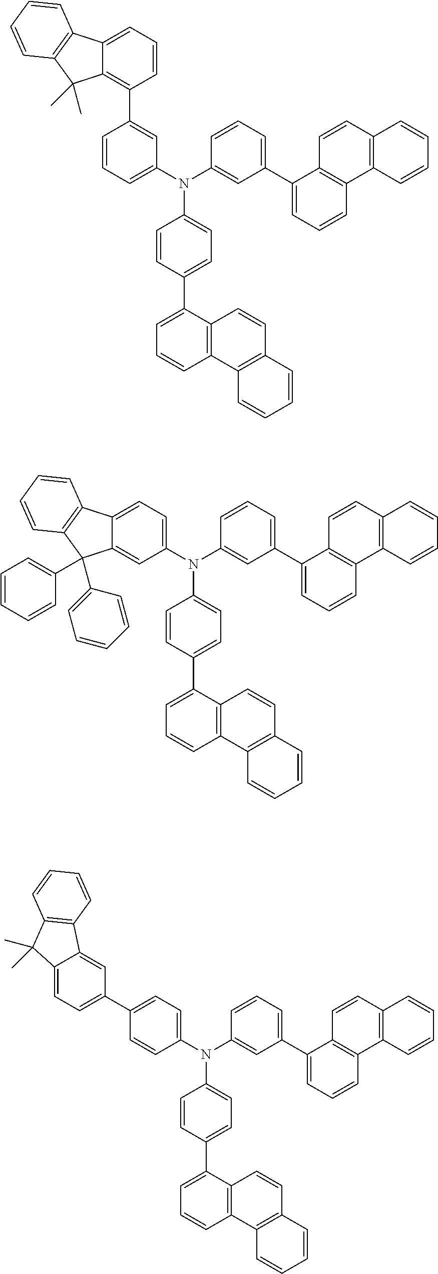 [SMILES: CC1(C)c2ccccc2-c2cccc(-c3cccc(N(c4ccc(-c5cccc6c5ccc5ccccc56)cc4)c4cccc(-c5cccc6c5ccc5ccccc56)c4)c3)c21 ; c1ccc(C2(c3ccccc3)c3ccccc3-c3ccc(N(c4ccc(-c5cccc6c5ccc5ccccc56)cc4)c4cccc(-c5cccc6c5ccc5ccccc56)c4)cc32)cc1 ; CC1(C)c2ccccc2-c2cc(-c3ccc(N(c4ccc(-c5cccc6c5ccc5ccccc56)cc4)c4cccc(-c5cccc6c5ccc5ccccc56)c4)cc3)ccc21]

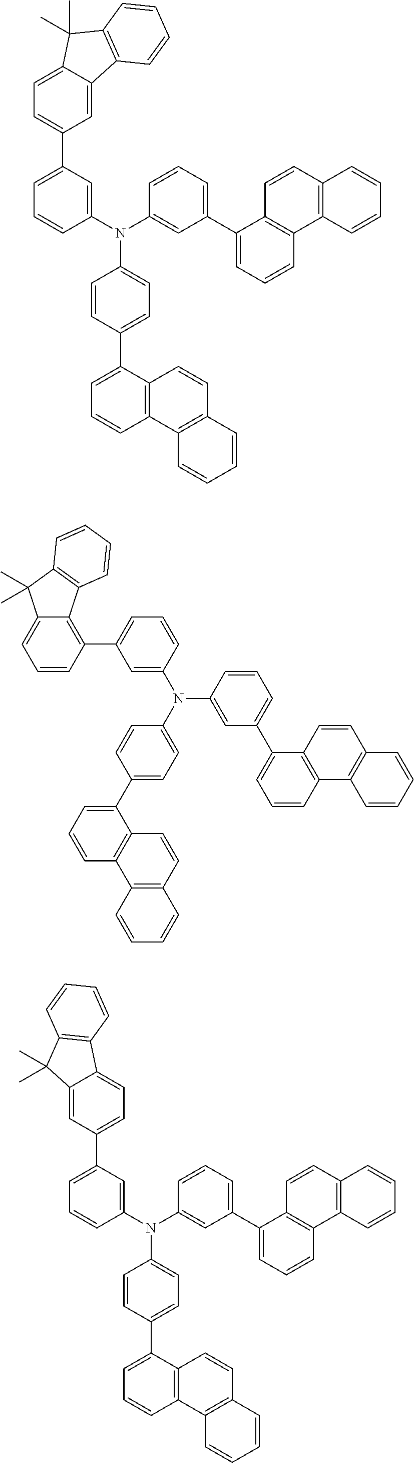 [SMILES: CC1(C)c2ccccc2-c2cc(-c3cccc(N(c4ccc(-c5cccc6c5ccc5ccccc56)cc4)c4cccc(-c5cccc6c5ccc5ccccc56)c4)c3)ccc21 ; CC1(C)c2ccccc2-c2c(-c3cccc(N(c4ccc(-c5cccc6c5ccc5ccccc56)cc4)c4cccc(-c5cccc6c5ccc5ccccc56)c4)c3)cccc21 ; CC1(C)c2ccccc2-c2ccc(-c3cccc(N(c4ccc(-c5cccc6c5ccc5ccccc56)cc4)c4cccc(-c5cccc6c5ccc5ccccc56)c4)c3)cc21]

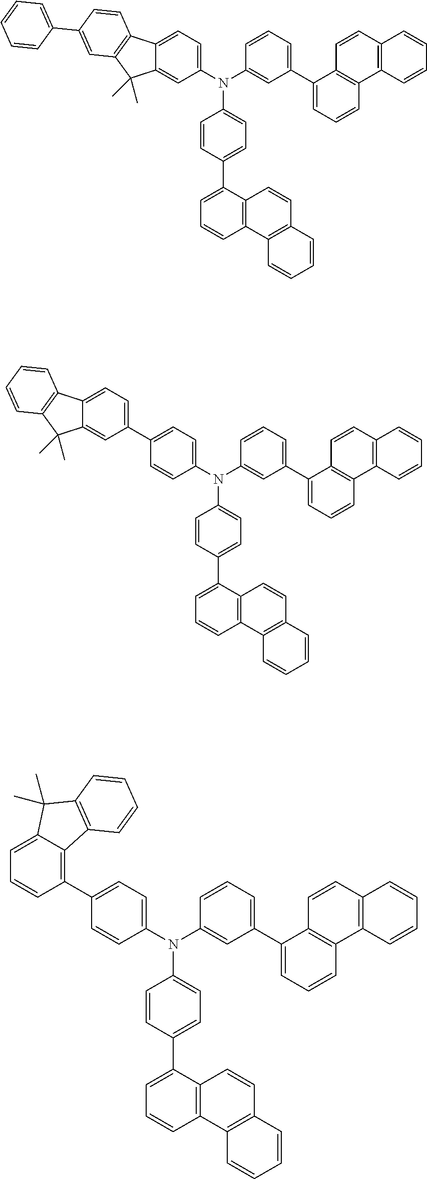 [SMILES: CC1(C)c2cc(-c3ccccc3)ccc2-c2ccc(N(c3ccc(-c4cccc5c4ccc4ccccc45)cc3)c3cccc(-c4cccc5c4ccc4ccccc45)c3)cc21 ; CC1(C)c2ccccc2-c2ccc(-c3ccc(N(c4ccc(-c5cccc6c5ccc5ccccc56)cc4)c4cccc(-c5cccc6c5ccc5ccccc56)c4)cc3)cc21 ; CC1(C)c2ccccc2-c2c(-c3ccc(N(c4ccc(-c5cccc6c5ccc5ccccc56)cc4)c4cccc(-c5cccc6c5ccc5ccccc56)c4)cc3)cccc21]

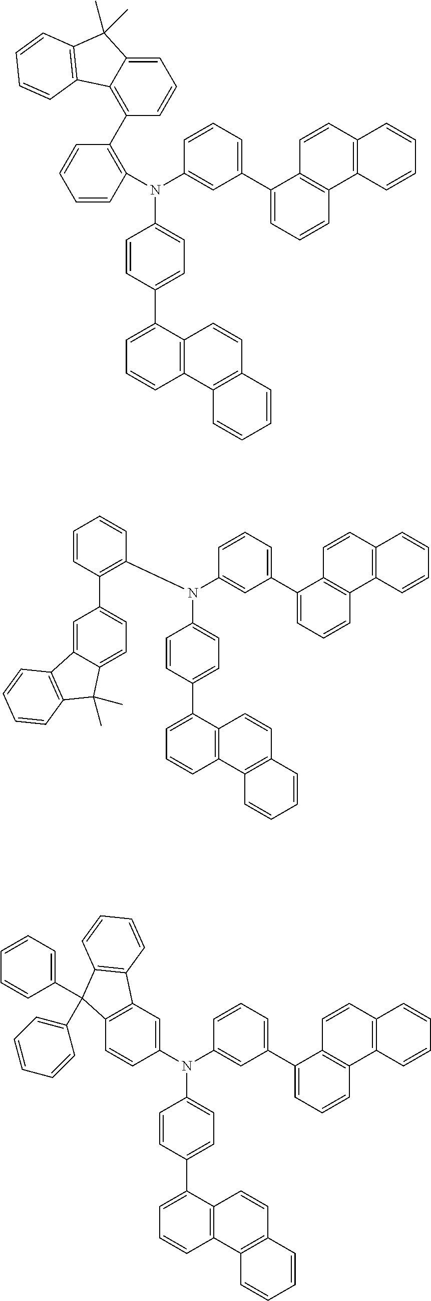 [SMILES: CC1(C)c2ccccc2-c2c(-c3ccccc3N(c3ccc(-c4cccc5c4ccc4ccccc45)cc3)c3cccc(-c4cccc5c4ccc4ccccc45)c3)cccc21 ; CC1(C)c2ccccc2-c2cc(-c3ccccc3N(c3ccc(-c4cccc5c4ccc4ccccc45)cc3)c3cccc(-c4cccc5c4ccc4ccccc45)c3)ccc21 ; c1ccc(C2(c3ccccc3)c3ccccc3-c3cc(N(c4ccc(-c5cccc6c5ccc5ccccc56)cc4)c4cccc(-c5cccc6c5ccc5ccccc56)c4)ccc32)cc1]

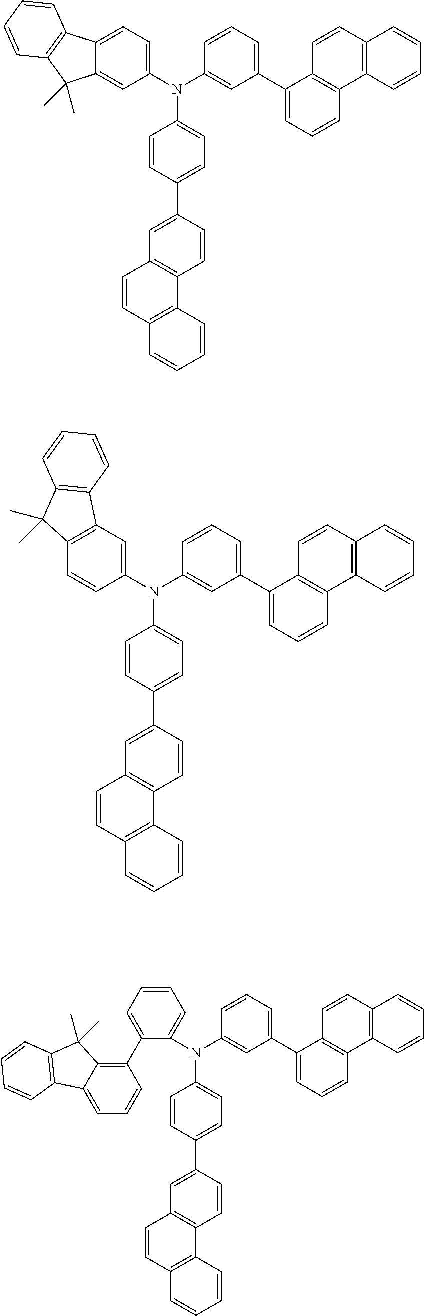 [SMILES: CC1(C)c2ccccc2-c2ccc(N(c3ccc(-c4ccc5c(ccc6ccccc65)c4)cc3)c3cccc(-c4cccc5c4ccc4ccccc45)c3)cc21 ; CC1(C)c2ccccc2-c2cc(N(c3ccc(-c4ccc5c(ccc6ccccc65)c4)cc3)c3cccc(-c4cccc5c4ccc4ccccc45)c3)ccc21 ; CC1(C)c2ccccc2-c2cccc(-c3ccccc3N(c3ccc(-c4ccc5c(ccc6ccccc65)c4)cc3)c3cccc(-c4cccc5c4ccc4ccccc45)c3)c21]

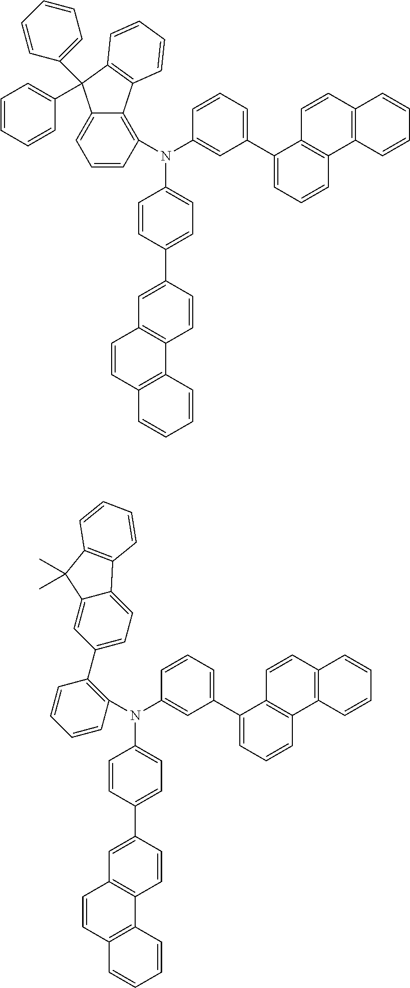 [SMILES: c1ccc(C2(c3ccccc3)c3ccccc3-c3c(N(c4ccc(-c5ccc6c(ccc7ccccc76)c5)cc4)c4cccc(-c5cccc6c5ccc5ccccc56)c4)cccc32)cc1 ; CC1(C)c2ccccc2-c2ccc(-c3ccccc3N(c3ccc(-c4ccc5c(ccc6ccccc65)c4)cc3)c3cccc(-c4cccc5c4ccc4ccccc45)c3)cc21]

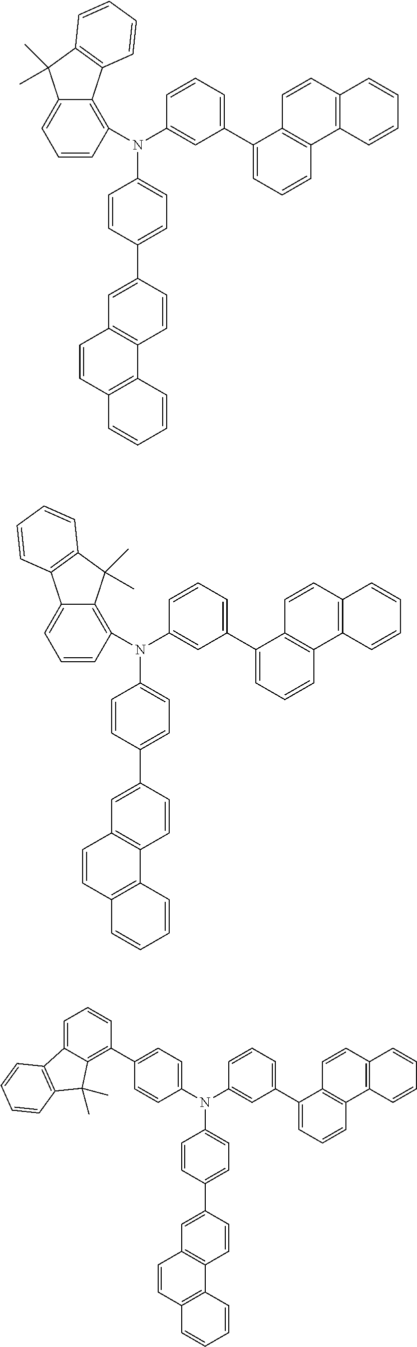 [SMILES: CC1(C)c2ccccc2-c2c(N(c3ccc(-c4ccc5c(ccc6ccccc65)c4)cc3)c3cccc(-c4cccc5c4ccc4ccccc45)c3)cccc21 ; CC1(C)c2ccccc2-c2cccc(N(c3ccc(-c4ccc5c(ccc6ccccc65)c4)cc3)c3cccc(-c4cccc5c4ccc4ccccc45)c3)c21 ; CC1(C)c2ccccc2-c2cccc(-c3ccc(N(c4ccc(-c5ccc6c(ccc7ccccc76)c5)cc4)c4cccc(-c5cccc6c5ccc5ccccc56)c4)cc3)c21]

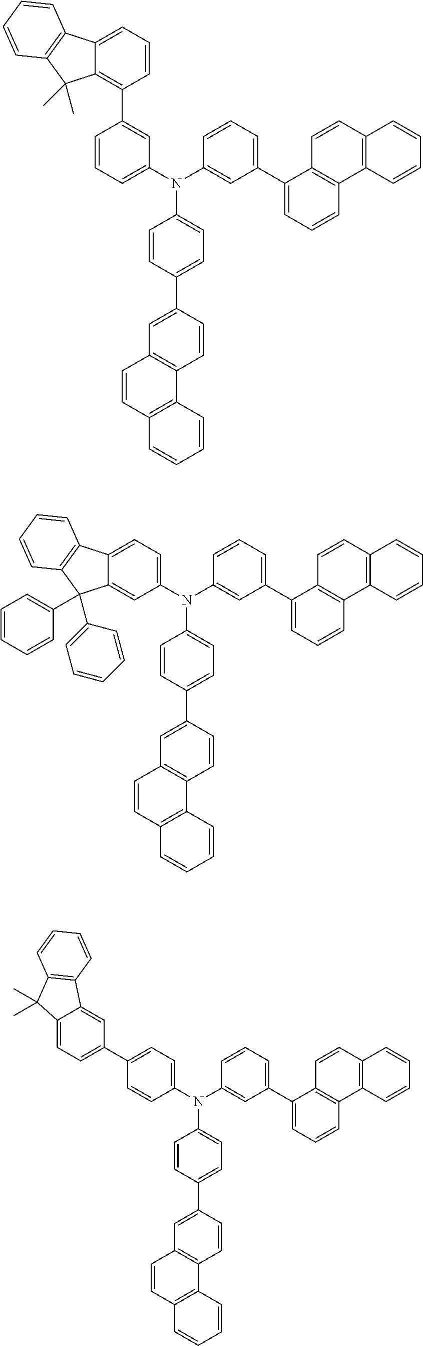 [SMILES: CC1(C)c2ccccc2-c2cccc(-c3cccc(N(c4ccc(-c5ccc6c(ccc7ccccc76)c5)cc4)c4cccc(-c5cccc6c5ccc5ccccc56)c4)c3)c21 ; c1ccc(C2(c3ccccc3)c3ccccc3-c3ccc(N(c4ccc(-c5ccc6c(ccc7ccccc76)c5)cc4)c4cccc(-c5cccc6c5ccc5ccccc56)c4)cc32)cc1 ; CC1(C)c2ccccc2-c2cc(-c3ccc(N(c4ccc(-c5ccc6c(ccc7ccccc76)c5)cc4)c4cccc(-c5cccc6c5ccc5ccccc56)c4)cc3)ccc21]

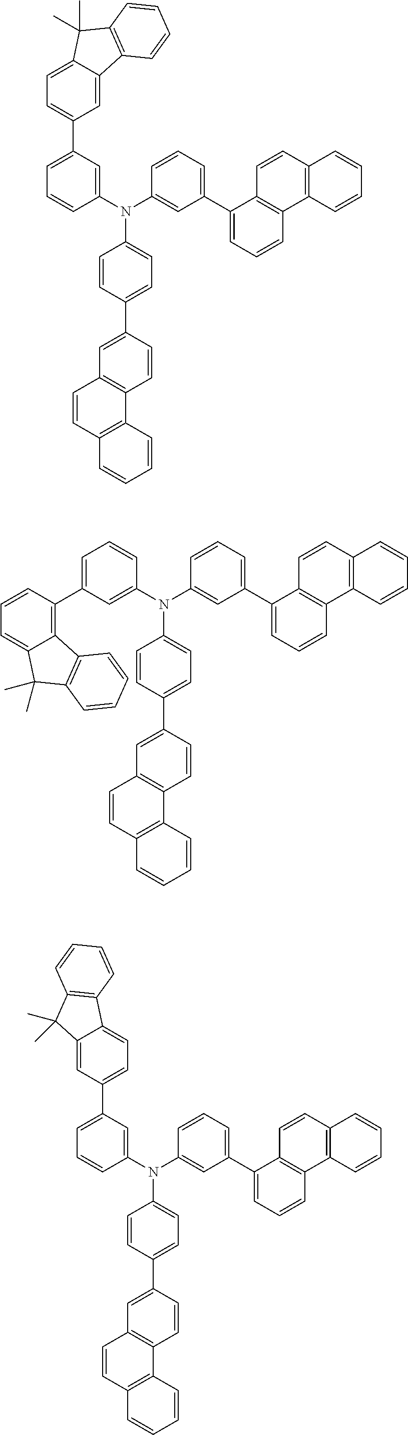 [SMILES: CC1(C)c2ccccc2-c2cc(-c3cccc(N(c4ccc(-c5ccc6c(ccc7ccccc76)c5)cc4)c4cccc(-c5cccc6c5ccc5ccccc56)c4)c3)ccc21 ; CC1(C)c2ccccc2-c2c(-c3cccc(N(c4ccc(-c5ccc6c(ccc7ccccc76)c5)cc4)c4cccc(-c5cccc6c5ccc5ccccc56)c4)c3)cccc21 ; CC1(C)c2ccccc2-c2ccc(-c3cccc(N(c4ccc(-c5ccc6c(ccc7ccccc76)c5)cc4)c4cccc(-c5cccc6c5ccc5ccccc56)c4)c3)cc21]

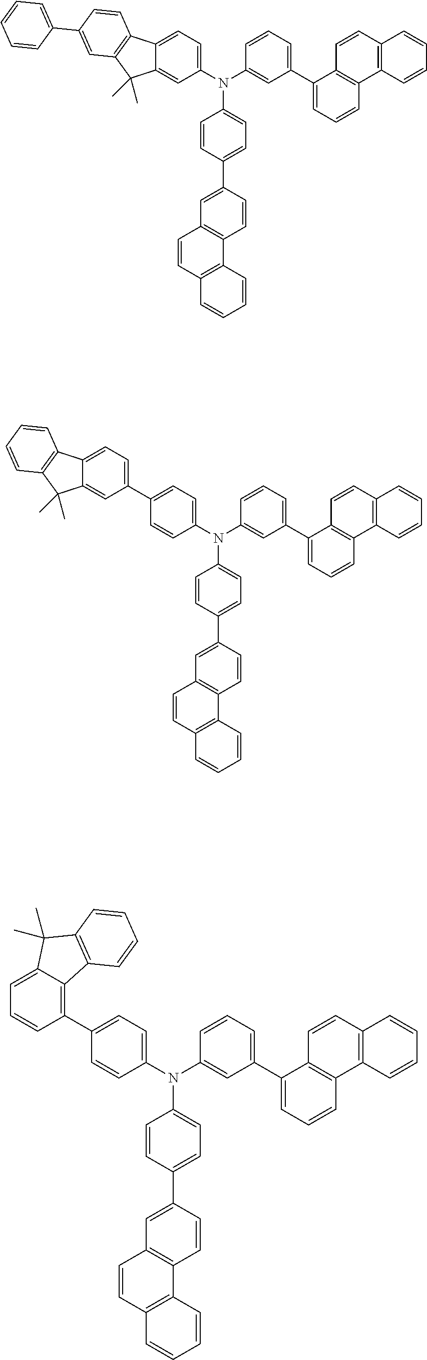 [SMILES: CC1(C)c2cc(-c3ccccc3)ccc2-c2ccc(N(c3ccc(-c4ccc5c(ccc6ccccc65)c4)cc3)c3cccc(-c4cccc5c4ccc4ccccc45)c3)cc21 ; CC1(C)c2ccccc2-c2ccc(-c3ccc(N(c4ccc(-c5ccc6c(ccc7ccccc76)c5)cc4)c4cccc(-c5cccc6c5ccc5ccccc56)c4)cc3)cc21 ; CC1(C)c2ccccc2-c2c(-c3ccc(N(c4ccc(-c5ccc6c(ccc7ccccc76)c5)cc4)c4cccc(-c5cccc6c5ccc5ccccc56)c4)cc3)cccc21]

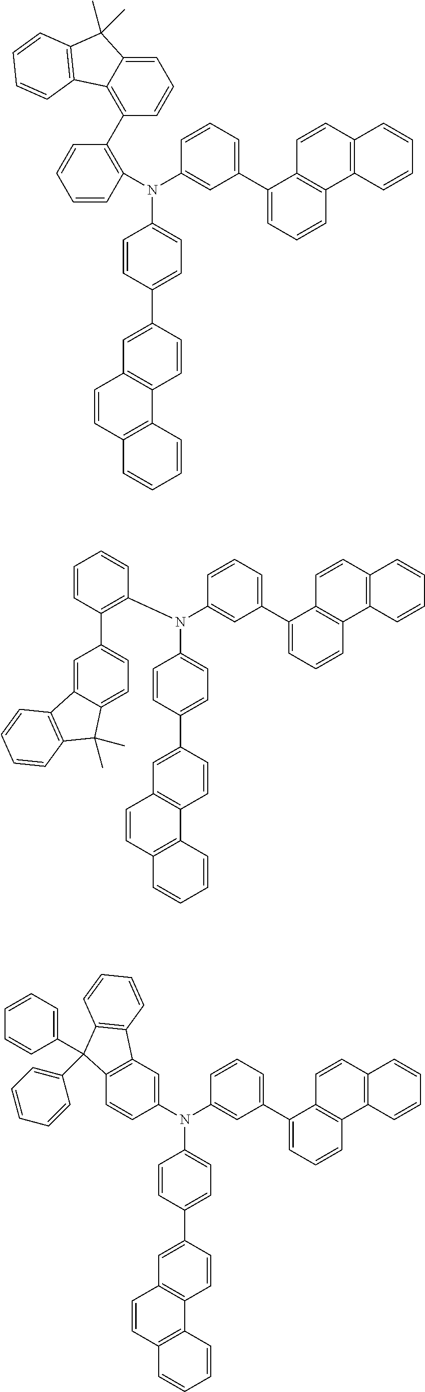 [SMILES: CC1(C)c2ccccc2-c2c(-c3ccccc3N(c3ccc(-c4ccc5c(ccc6ccccc65)c4)cc3)c3cccc(-c4cccc5c4ccc4ccccc45)c3)cccc21 ; CC1(C)c2ccccc2-c2cc(-c3ccccc3N(c3ccc(-c4ccc5c(ccc6ccccc65)c4)cc3)c3cccc(-c4cccc5c4ccc4ccccc45)c3)ccc21 ; c1ccc(C2(c3ccccc3)c3ccccc3-c3cc(N(c4ccc(-c5ccc6c(ccc7ccccc76)c5)cc4)c4cccc(-c5cccc6c5ccc5ccccc56)c4)ccc32)cc1]

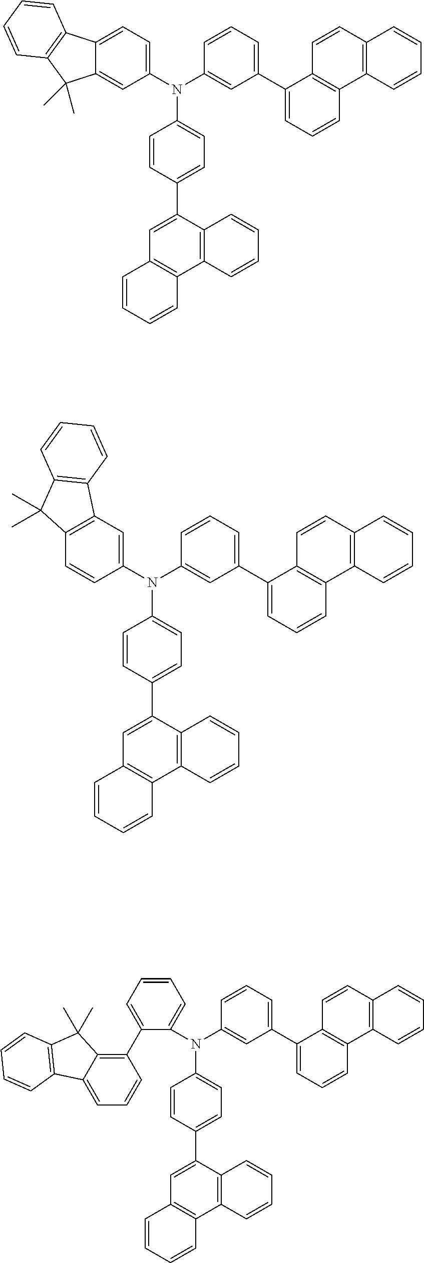 [SMILES: CC1(C)c2ccccc2-c2ccc(N(c3ccc(-c4cc5ccccc5c5ccccc45)cc3)c3cccc(-c4cccc5c4ccc4ccccc45)c3)cc21 ; CC1(C)c2ccccc2-c2cc(N(c3ccc(-c4cc5ccccc5c5ccccc45)cc3)c3cccc(-c4cccc5c4ccc4ccccc45)c3)ccc21 ; CC1(C)c2ccccc2-c2cccc(-c3ccccc3N(c3ccc(-c4cc5ccccc5c5ccccc45)cc3)c3cccc(-c4cccc5c4ccc4ccccc45)c3)c21]

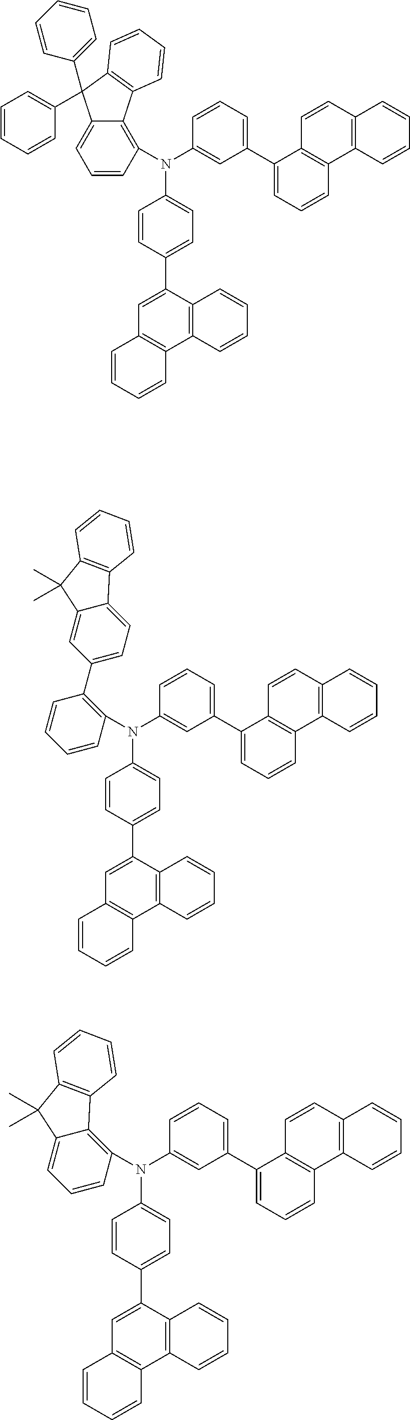 [SMILES: c1ccc(C2(c3ccccc3)c3ccccc3-c3c(N(c4ccc(-c5cc6ccccc6c6ccccc56)cc4)c4cccc(-c5cccc6c5ccc5ccccc56)c4)cccc32)cc1 ; CC1(C)c2ccccc2-c2ccc(-c3ccccc3N(c3ccc(-c4cc5ccccc5c5ccccc45)cc3)c3cccc(-c4cccc5c4ccc4ccccc45)c3)cc21 ; CC1(C)c2ccccc2-c2c(N(c3ccc(-c4cc5ccccc5c5ccccc45)cc3)c3cccc(-c4cccc5c4ccc4ccccc45)c3)cccc21]

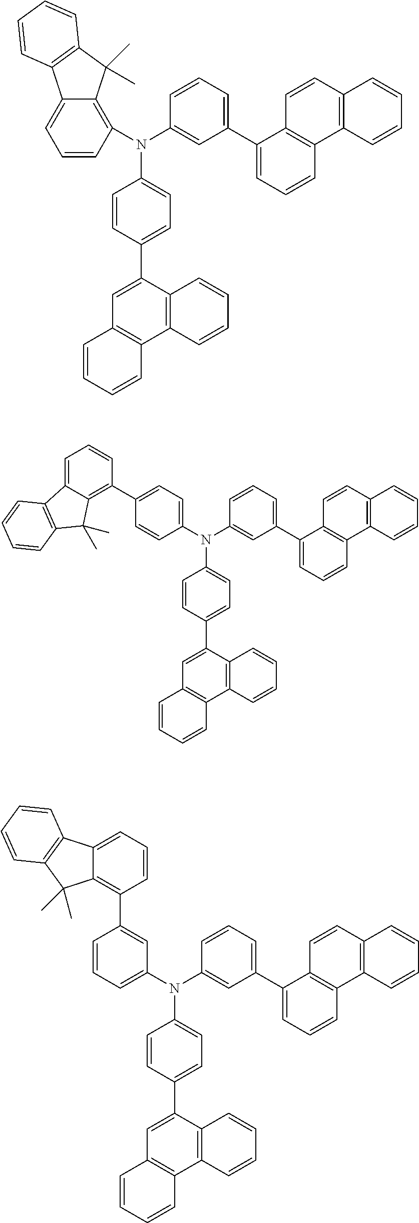 [SMILES: CC1(C)c2ccccc2-c2cccc(N(c3ccc(-c4cc5ccccc5c5ccccc45)cc3)c3cccc(-c4cccc5c4ccc4ccccc45)c3)c21 ; CC1(C)c2ccccc2-c2cccc(-c3ccc(N(c4ccc(-c5cc6ccccc6c6ccccc56)cc4)c4cccc(-c5cccc6c5ccc5ccccc56)c4)cc3)c21 ; CC1(C)c2ccccc2-c2cccc(-c3cccc(N(c4ccc(-c5cc6ccccc6c6ccccc56)cc4)c4cccc(-c5cccc6c5ccc5ccccc56)c4)c3)c21]

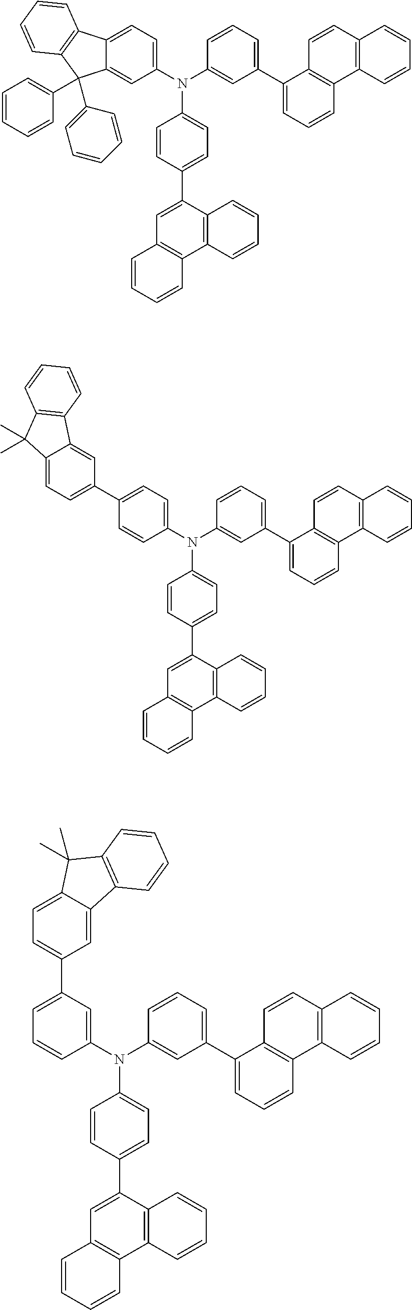 [SMILES: c1ccc(C2(c3ccccc3)c3ccccc3-c3ccc(N(c4ccc(-c5cc6ccccc6c6ccccc56)cc4)c4cccc(-c5cccc6c5ccc5ccccc56)c4)cc32)cc1 ; CC1(C)c2ccccc2-c2cc(-c3ccc(N(c4ccc(-c5cc6ccccc6c6ccccc56)cc4)c4cccc(-c5cccc6c5ccc5ccccc56)c4)cc3)ccc21 ; CC1(C)c2ccccc2-c2cc(-c3cccc(N(c4ccc(-c5cc6ccccc6c6ccccc56)cc4)c4cccc(-c5cccc6c5ccc5ccccc56)c4)c3)ccc21]

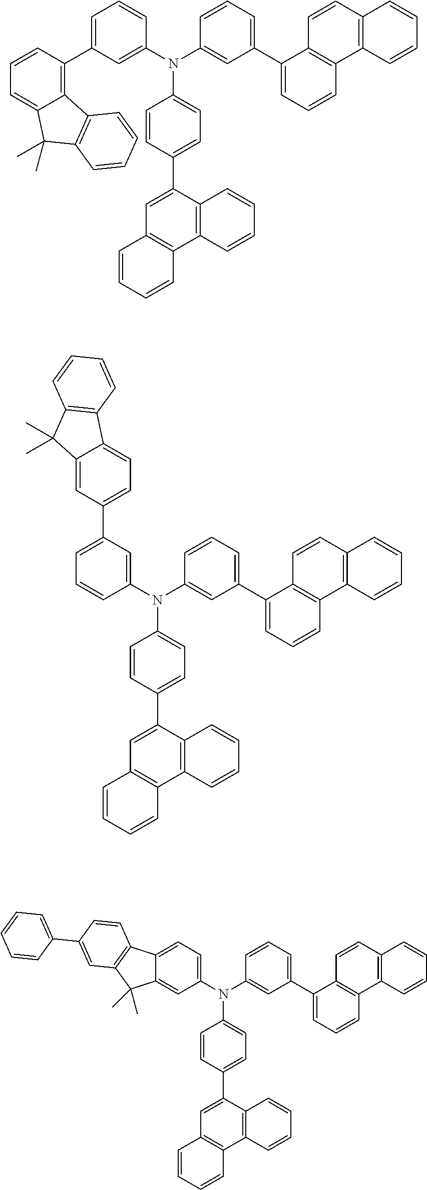 [SMILES: CC1(C)c2ccccc2-c2c(-c3cccc(N(c4ccc(-c5cc6ccccc6c6ccccc56)cc4)c4cccc(-c5cccc6c5ccc5ccccc56)c4)c3)cccc21 ; CC1(C)c2ccccc2-c2ccc(-c3cccc(N(c4ccc(-c5cc6ccccc6c6ccccc56)cc4)c4cccc(-c5cccc6c5ccc5ccccc56)c4)c3)cc21 ; CC1(C)c2cc(-c3ccccc3)ccc2-c2ccc(N(c3ccc(-c4cc5ccccc5c5ccccc45)cc3)c3cccc(-c4cccc5c4ccc4ccccc45)c3)cc21]

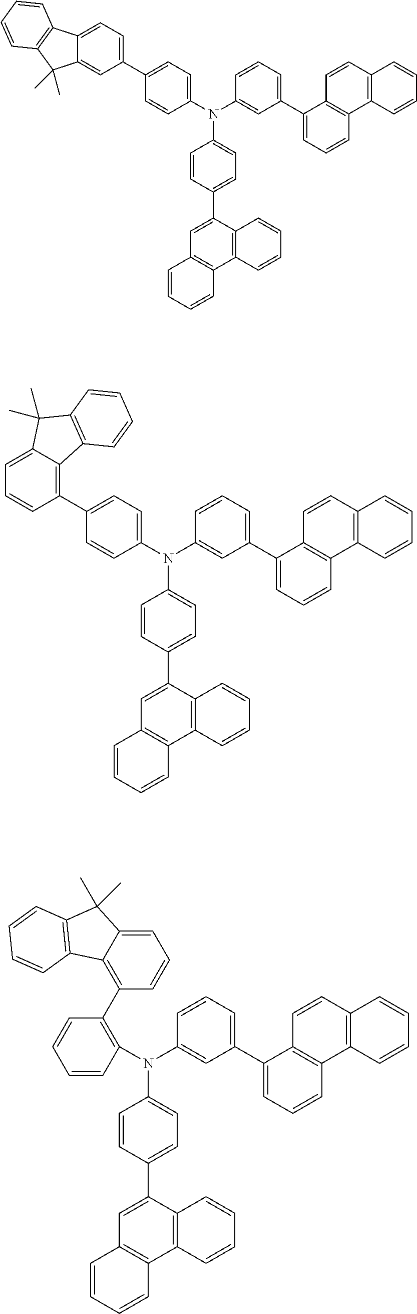 [SMILES: CC1(C)c2ccccc2-c2ccc(-c3ccc(N(c4ccc(-c5cc6ccccc6c6ccccc56)cc4)c4cccc(-c5cccc6c5ccc5ccccc56)c4)cc3)cc21 ; CC1(C)c2ccccc2-c2c(-c3ccc(N(c4ccc(-c5cc6ccccc6c6ccccc56)cc4)c4cccc(-c5cccc6c5ccc5ccccc56)c4)cc3)cccc21 ; CC1(C)c2ccccc2-c2c(-c3ccccc3N(c3ccc(-c4cc5ccccc5c5ccccc45)cc3)c3cccc(-c4cccc5c4ccc4ccccc45)c3)cccc21]

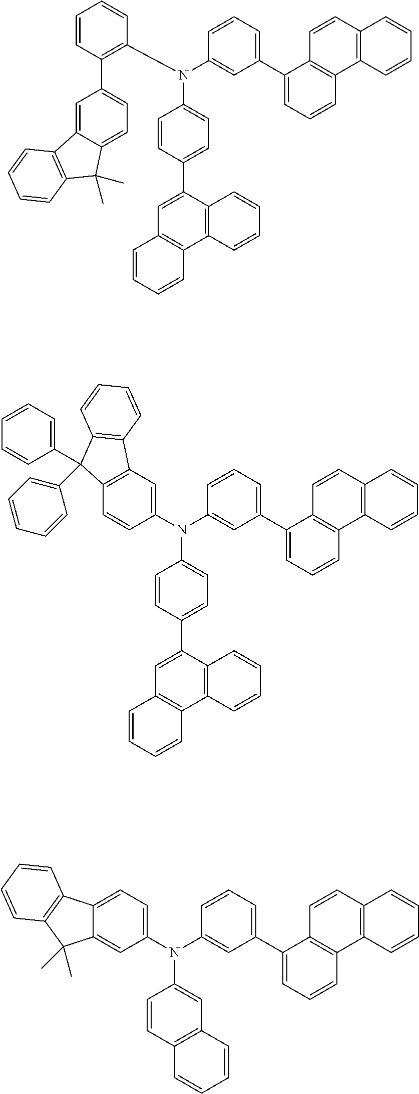 [SMILES: CC1(C)c2ccccc2-c2cc(-c3ccccc3N(c3ccc(-c4cc5ccccc5c5ccccc45)cc3)c3cccc(-c4cccc5c4ccc4ccccc45)c3)ccc21 ; c1ccc(C2(c3ccccc3)c3ccccc3-c3cc(N(c4ccc(-c5cc6ccccc6c6ccccc56)cc4)c4cccc(-c5cccc6c5ccc5ccccc56)c4)ccc32)cc1 ; CC1(C)c2ccccc2-c2ccc(N(c3cccc(-c4cccc5c4ccc4ccccc45)c3)c3ccc4ccccc4c3)cc21]

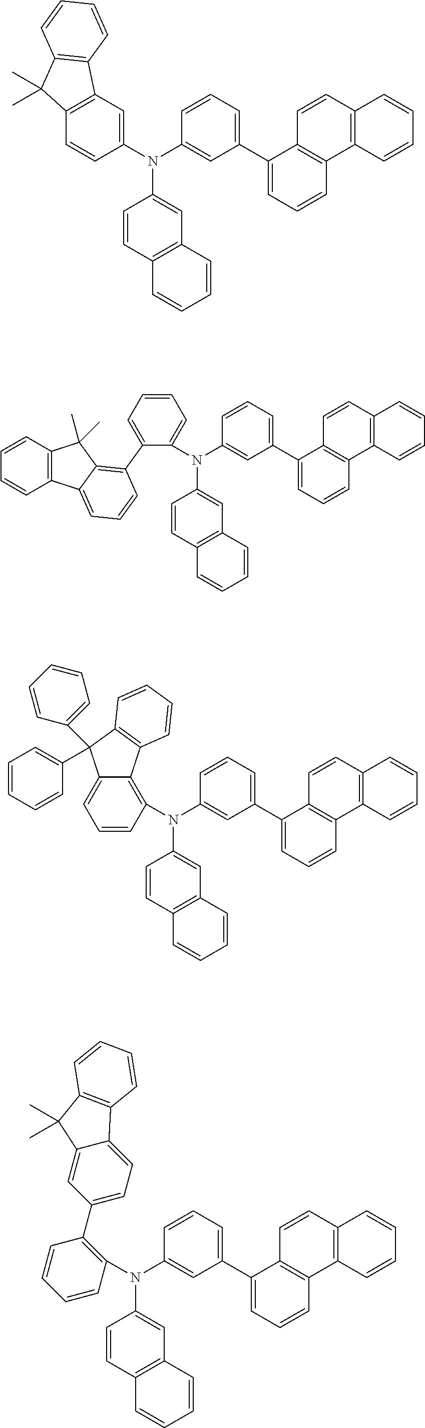 [SMILES: CC1(C)c2ccccc2-c2cc(N(c3cccc(-c4cccc5c4ccc4ccccc45)c3)c3ccc4ccccc4c3)ccc21 ; CC1(C)c2ccccc2-c2cccc(-c3ccccc3N(c3cccc(-c4cccc5c4ccc4ccccc45)c3)c3ccc4ccccc4c3)c21 ; c1ccc(C2(c3ccccc3)c3ccccc3-c3c(N(c4cccc(-c5cccc6c5ccc5ccccc56)c4)c4ccc5ccccc5c4)cccc32)cc1 ; CC1(C)c2ccccc2-c2ccc(-c3ccccc3N(c3cccc(-c4cccc5c4ccc4ccccc45)c3)c3ccc4ccccc4c3)cc21]

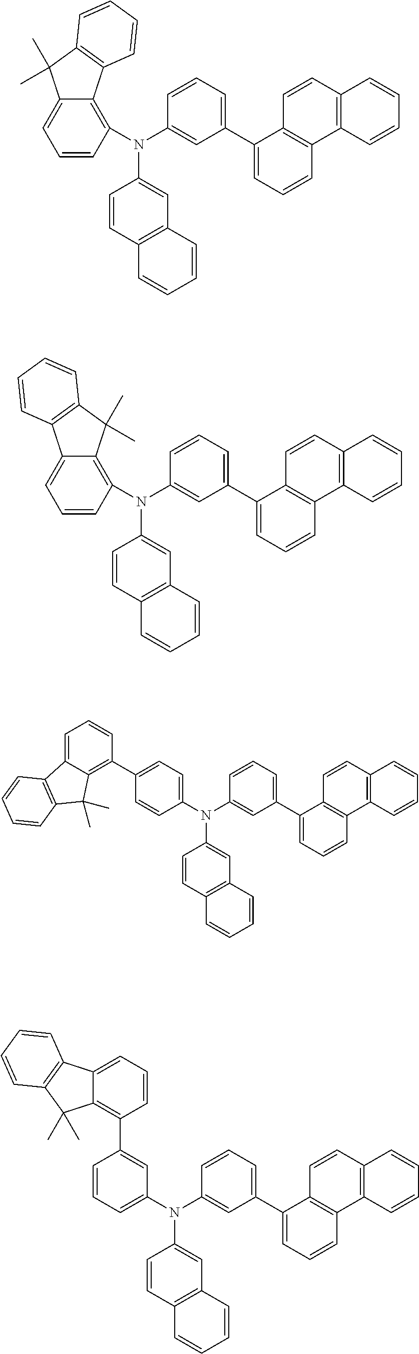 [SMILES: CC1(C)c2ccccc2-c2c(N(c3cccc(-c4cccc5c4ccc4ccccc45)c3)c3ccc4ccccc4c3)cccc21 ; CC1(C)c2ccccc2-c2cccc(N(c3cccc(-c4cccc5c4ccc4ccccc45)c3)c3ccc4ccccc4c3)c21 ; CC1(C)c2ccccc2-c2cccc(-c3ccc(N(c4cccc(-c5cccc6c5ccc5ccccc56)c4)c4ccc5ccccc5c4)cc3)c21 ; CC1(C)c2ccccc2-c2cccc(-c3cccc(N(c4cccc(-c5cccc6c5ccc5ccccc56)c4)c4ccc5ccccc5c4)c3)c21]

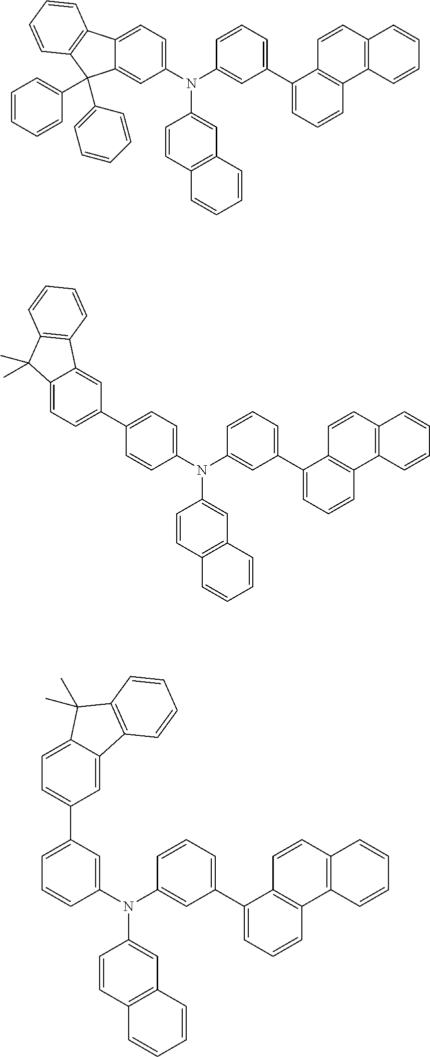 [SMILES: c1ccc(C2(c3ccccc3)c3ccccc3-c3ccc(N(c4cccc(-c5cccc6c5ccc5ccccc56)c4)c4ccc5ccccc5c4)cc32)cc1 ; CC1(C)c2ccccc2-c2cc(-c3ccc(N(c4cccc(-c5cccc6c5ccc5ccccc56)c4)c4ccc5ccccc5c4)cc3)ccc21 ; CC1(C)c2ccccc2-c2cc(-c3cccc(N(c4cccc(-c5cccc6c5ccc5ccccc56)c4)c4ccc5ccccc5c4)c3)ccc21]

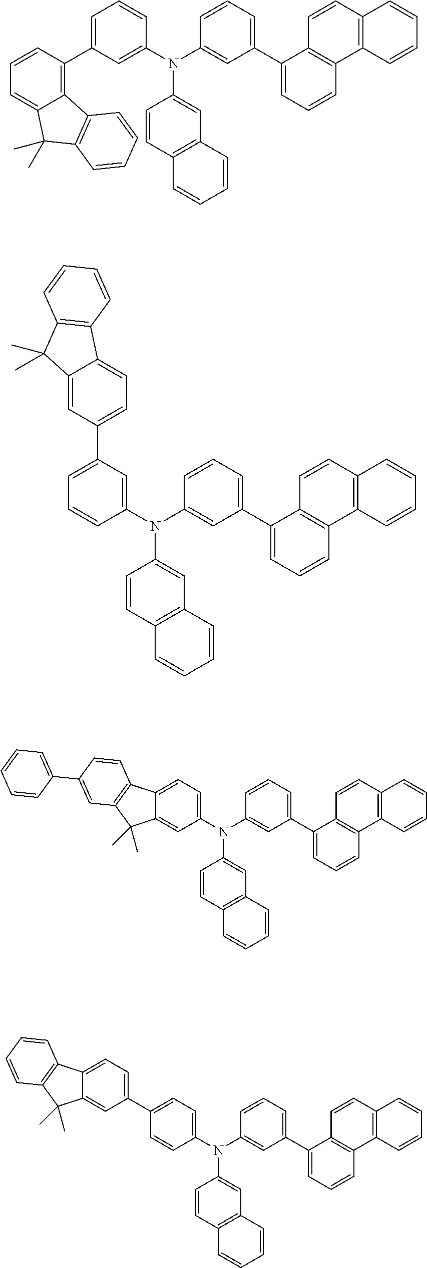 [SMILES: CC1(C)c2ccccc2-c2c(-c3cccc(N(c4cccc(-c5cccc6c5ccc5ccccc56)c4)c4ccc5ccccc5c4)c3)cccc21 ; CC1(C)c2ccccc2-c2ccc(-c3cccc(N(c4cccc(-c5cccc6c5ccc5ccccc56)c4)c4ccc5ccccc5c4)c3)cc21 ; CC1(C)c2cc(-c3ccccc3)ccc2-c2ccc(N(c3cccc(-c4cccc5c4ccc4ccccc45)c3)c3ccc4ccccc4c3)cc21 ; CC1(C)c2ccccc2-c2ccc(-c3ccc(N(c4cccc(-c5cccc6c5ccc5ccccc56)c4)c4ccc5ccccc5c4)cc3)cc21]

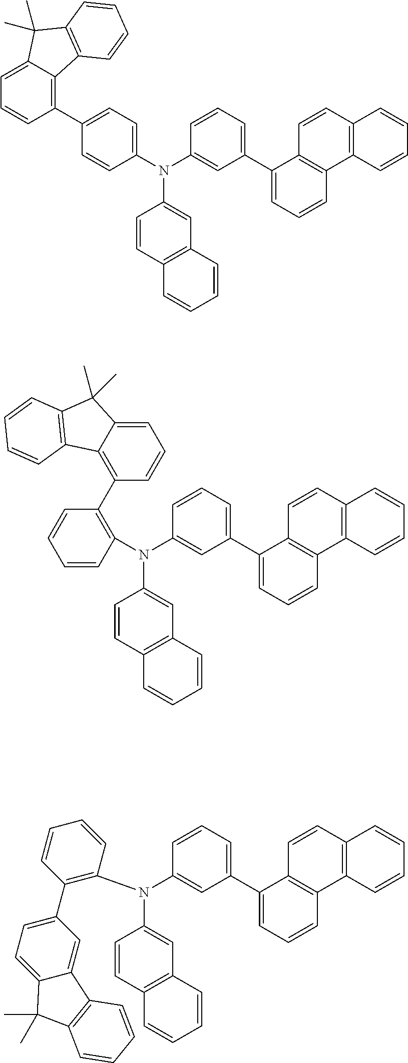 [SMILES: CC1(C)c2ccccc2-c2c(-c3ccc(N(c4cccc(-c5cccc6c5ccc5ccccc56)c4)c4ccc5ccccc5c4)cc3)cccc21 ; CC1(C)c2ccccc2-c2c(-c3ccccc3N(c3cccc(-c4cccc5c4ccc4ccccc45)c3)c3ccc4ccccc4c3)cccc21 ; CC1(C)c2ccccc2-c2cc(-c3ccccc3N(c3cccc(-c4cccc5c4ccc4ccccc45)c3)c3ccc4ccccc4c3)ccc21]

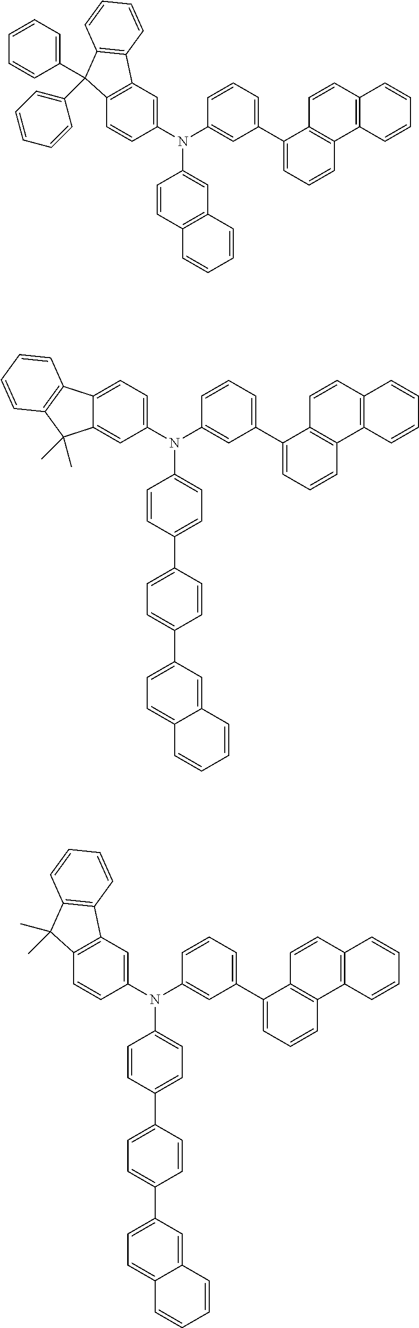 [SMILES: c1ccc(C2(c3ccccc3)c3ccccc3-c3cc(N(c4cccc(-c5cccc6c5ccc5ccccc56)c4)c4ccc5ccccc5c4)ccc32)cc1 ; CC1(C)c2ccccc2-c2ccc(N(c3ccc(-c4ccc(-c5ccc6ccccc6c5)cc4)cc3)c3cccc(-c4cccc5c4ccc4ccccc45)c3)cc21 ; CC1(C)c2ccccc2-c2cc(N(c3ccc(-c4ccc(-c5ccc6ccccc6c5)cc4)cc3)c3cccc(-c4cccc5c4ccc4ccccc45)c3)ccc21]

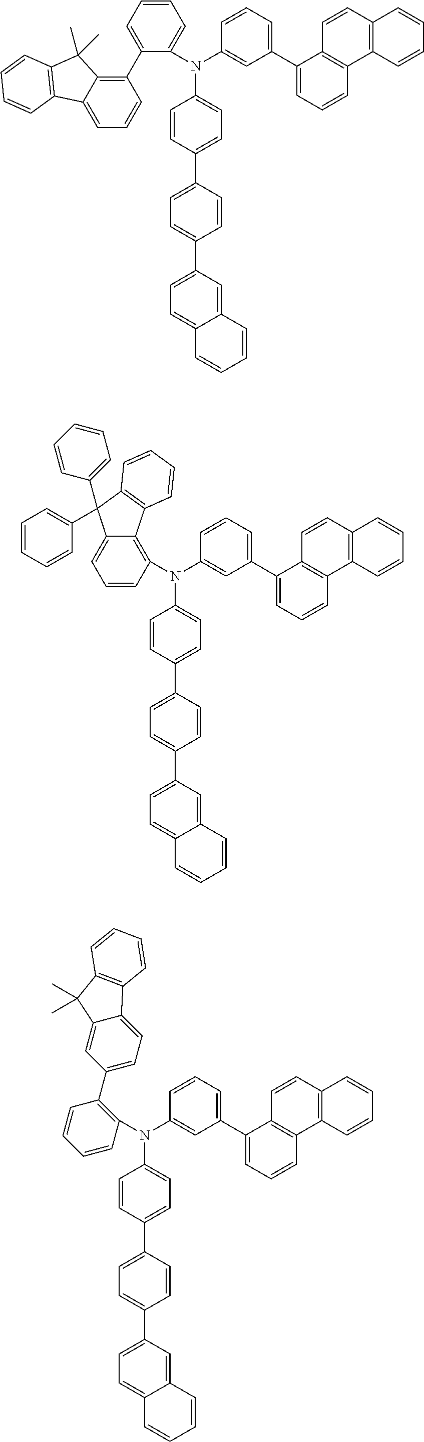 [SMILES: CC1(C)c2ccccc2-c2cccc(-c3ccccc3N(c3ccc(-c4ccc(-c5ccc6ccccc6c5)cc4)cc3)c3cccc(-c4cccc5c4ccc4ccccc45)c3)c21 ; c1ccc(C2(c3ccccc3)c3ccccc3-c3c(N(c4ccc(-c5ccc(-c6ccc7ccccc7c6)cc5)cc4)c4cccc(-c5cccc6c5ccc5ccccc56)c4)cccc32)cc1 ; CC1(C)c2ccccc2-c2ccc(-c3ccccc3N(c3ccc(-c4ccc(-c5ccc6ccccc6c5)cc4)cc3)c3cccc(-c4cccc5c4ccc4ccccc45)c3)cc21]

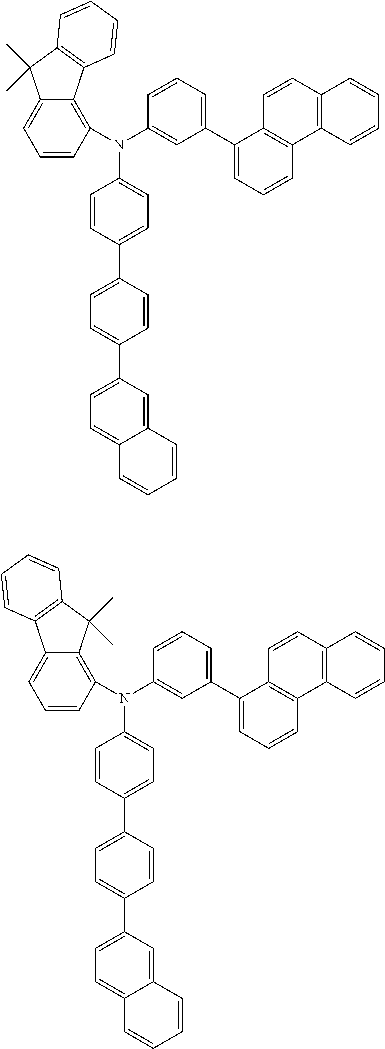 [SMILES: CC1(C)c2ccccc2-c2c(N(c3ccc(-c4ccc(-c5ccc6ccccc6c5)cc4)cc3)c3cccc(-c4cccc5c4ccc4ccccc45)c3)cccc21 ; CC1(C)c2ccccc2-c2cccc(N(c3ccc(-c4ccc(-c5ccc6ccccc6c5)cc4)cc3)c3cccc(-c4cccc5c4ccc4ccccc45)c3)c21]

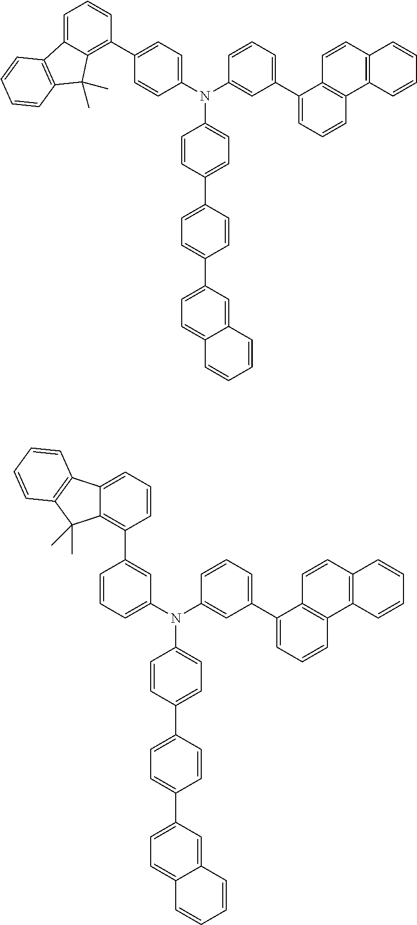 [SMILES: CC1(C)c2ccccc2-c2cccc(-c3ccc(N(c4ccc(-c5ccc(-c6ccc7ccccc7c6)cc5)cc4)c4cccc(-c5cccc6c5ccc5ccccc56)c4)cc3)c21 ; CC1(C)c2ccccc2-c2cccc(-c3cccc(N(c4ccc(-c5ccc(-c6ccc7ccccc7c6)cc5)cc4)c4cccc(-c5cccc6c5ccc5ccccc56)c4)c3)c21]

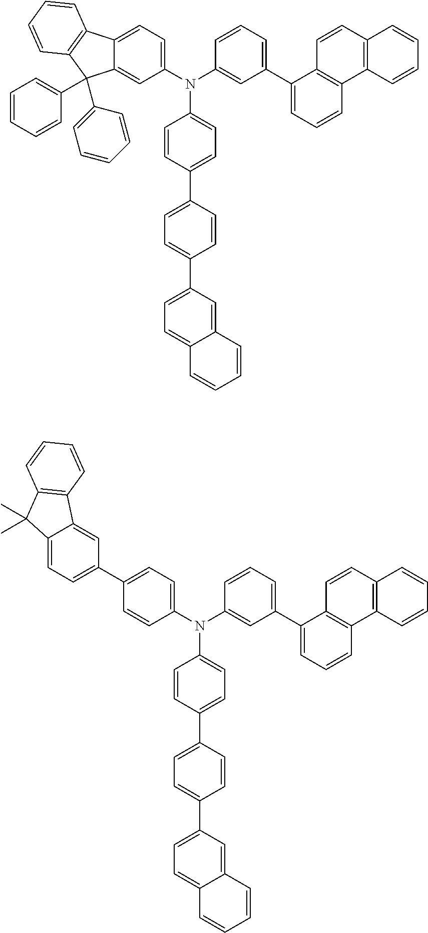 [SMILES: c1ccc(C2(c3ccccc3)c3ccccc3-c3ccc(N(c4ccc(-c5ccc(-c6ccc7ccccc7c6)cc5)cc4)c4cccc(-c5cccc6c5ccc5ccccc56)c4)cc32)cc1 ; CC1(C)c2ccccc2-c2cc(-c3ccc(N(c4ccc(-c5ccc(-c6ccc7ccccc7c6)cc5)cc4)c4cccc(-c5cccc6c5ccc5ccccc56)c4)cc3)ccc21]

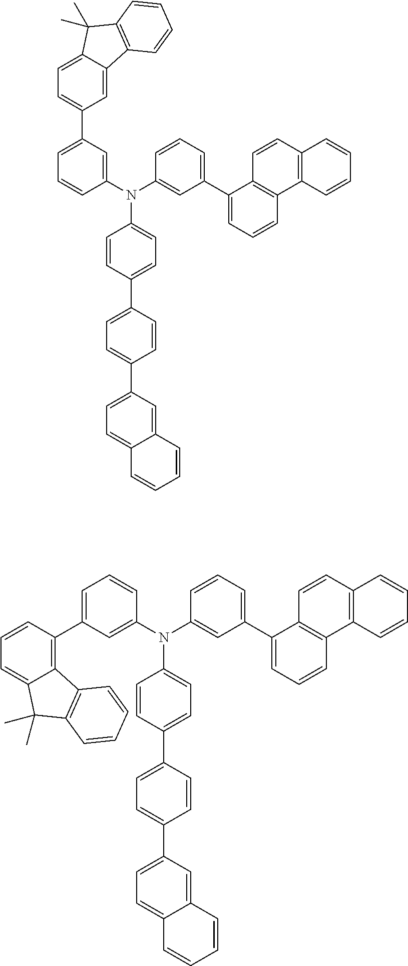 [SMILES: CC1(C)c2ccccc2-c2cc(-c3cccc(N(c4ccc(-c5ccc(-c6ccc7ccccc7c6)cc5)cc4)c4cccc(-c5cccc6c5ccc5ccccc56)c4)c3)ccc21 ; CC1(C)c2ccccc2-c2c(-c3cccc(N(c4ccc(-c5ccc(-c6ccc7ccccc7c6)cc5)cc4)c4cccc(-c5cccc6c5ccc5ccccc56)c4)c3)cccc21]

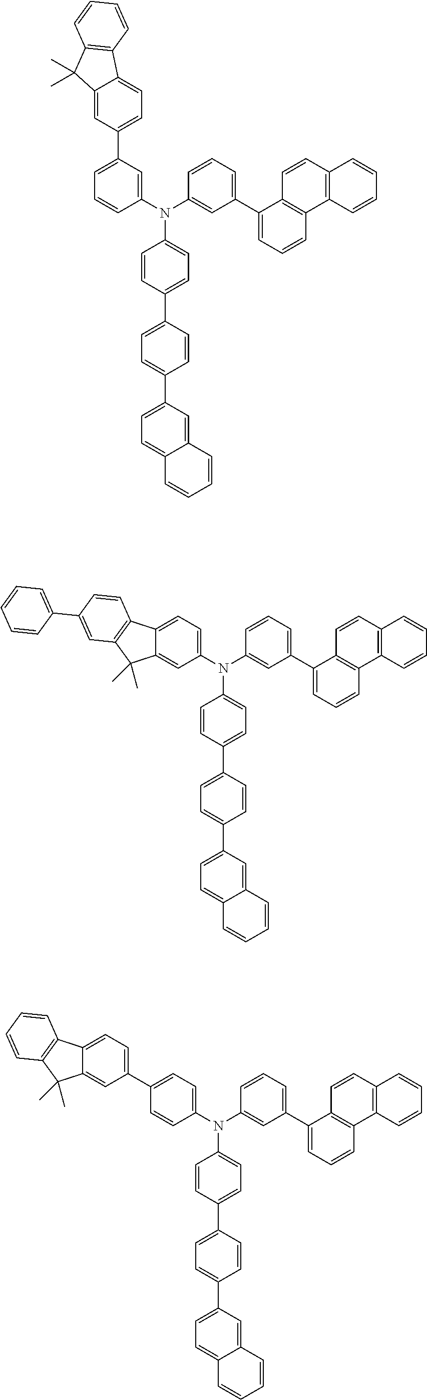 [SMILES: CC1(C)c2ccccc2-c2ccc(-c3cccc(N(c4ccc(-c5ccc(-c6ccc7ccccc7c6)cc5)cc4)c4cccc(-c5cccc6c5ccc5ccccc56)c4)c3)cc21 ; CC1(C)c2cc(-c3ccccc3)ccc2-c2ccc(N(c3ccc(-c4ccc(-c5ccc6ccccc6c5)cc4)cc3)c3cccc(-c4cccc5c4ccc4ccccc45)c3)cc21 ; CC1(C)c2ccccc2-c2ccc(-c3ccc(N(c4ccc(-c5ccc(-c6ccc7ccccc7c6)cc5)cc4)c4cccc(-c5cccc6c5ccc5ccccc56)c4)cc3)cc21]

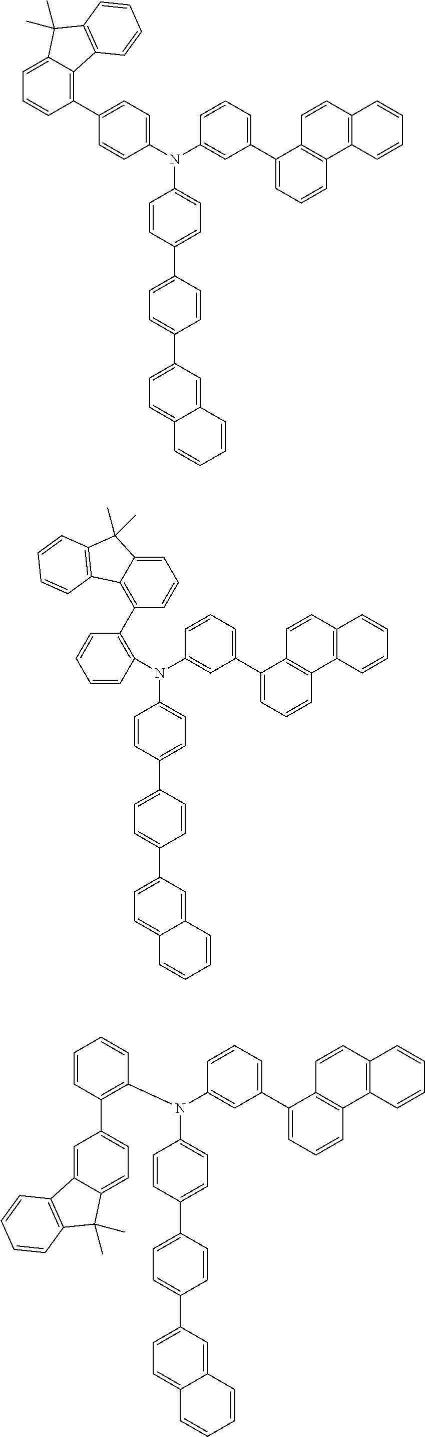 [SMILES: CC1(C)c2ccccc2-c2c(-c3ccc(N(c4ccc(-c5ccc(-c6ccc7ccccc7c6)cc5)cc4)c4cccc(-c5cccc6c5ccc5ccccc56)c4)cc3)cccc21 ; CC1(C)c2ccccc2-c2c(-c3ccccc3N(c3ccc(-c4ccc(-c5ccc6ccccc6c5)cc4)cc3)c3cccc(-c4cccc5c4ccc4ccccc45)c3)cccc21 ; CC1(C)c2ccccc2-c2cc(-c3ccccc3N(c3ccc(-c4ccc(-c5ccc6ccccc6c5)cc4)cc3)c3cccc(-c4cccc5c4ccc4ccccc45)c3)ccc21]

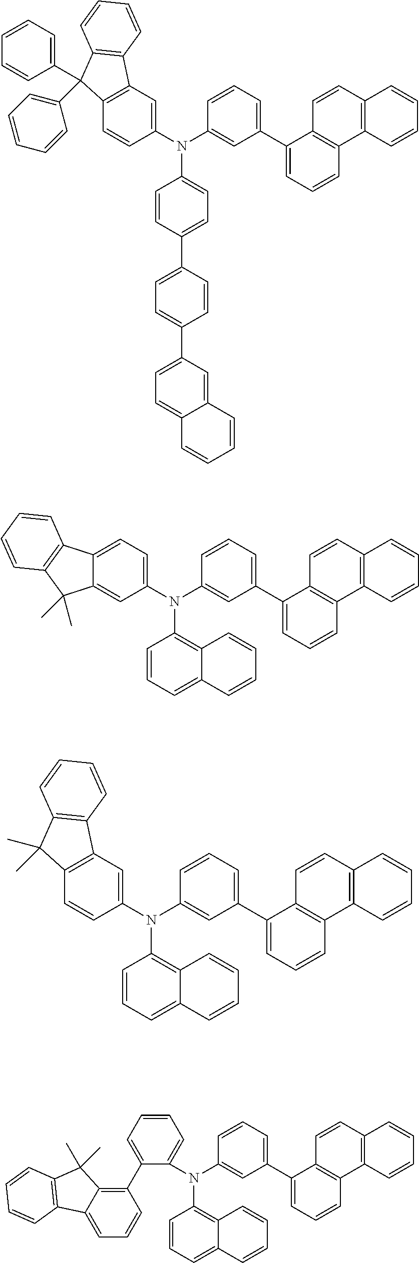 [SMILES: c1ccc(C2(c3ccccc3)c3ccccc3-c3cc(N(c4ccc(-c5ccc(-c6ccc7ccccc7c6)cc5)cc4)c4cccc(-c5cccc6c5ccc5ccccc56)c4)ccc32)cc1 ; CC1(C)c2ccccc2-c2ccc(N(c3cccc(-c4cccc5c4ccc4ccccc45)c3)c3cccc4ccccc34)cc21 ; CC1(C)c2ccccc2-c2cc(N(c3cccc(-c4cccc5c4ccc4ccccc45)c3)c3cccc4ccccc34)ccc21 ; CC1(C)c2ccccc2-c2cccc(-c3ccccc3N(c3cccc(-c4cccc5c4ccc4ccccc45)c3)c3cccc4ccccc34)c21]

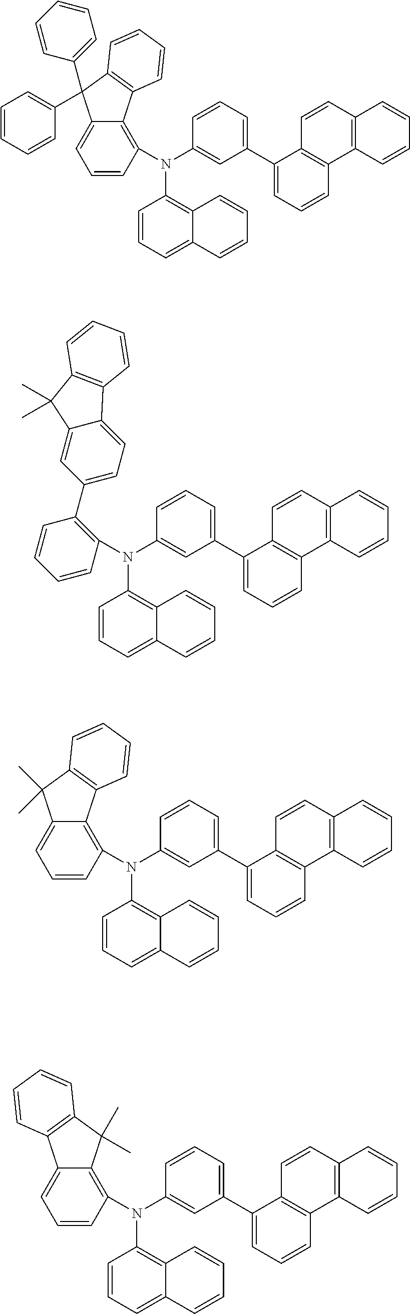 [SMILES: c1ccc(C2(c3ccccc3)c3ccccc3-c3c(N(c4cccc(-c5cccc6c5ccc5ccccc56)c4)c4cccc5ccccc45)cccc32)cc1 ; CC1(C)c2ccccc2-c2ccc(-c3ccccc3N(c3cccc(-c4cccc5c4ccc4ccccc45)c3)c3cccc4ccccc34)cc21 ; CC1(C)c2ccccc2-c2c(N(c3cccc(-c4cccc5c4ccc4ccccc45)c3)c3cccc4ccccc34)cccc21 ; CC1(C)c2ccccc2-c2cccc(N(c3cccc(-c4cccc5c4ccc4ccccc45)c3)c3cccc4ccccc34)c21]

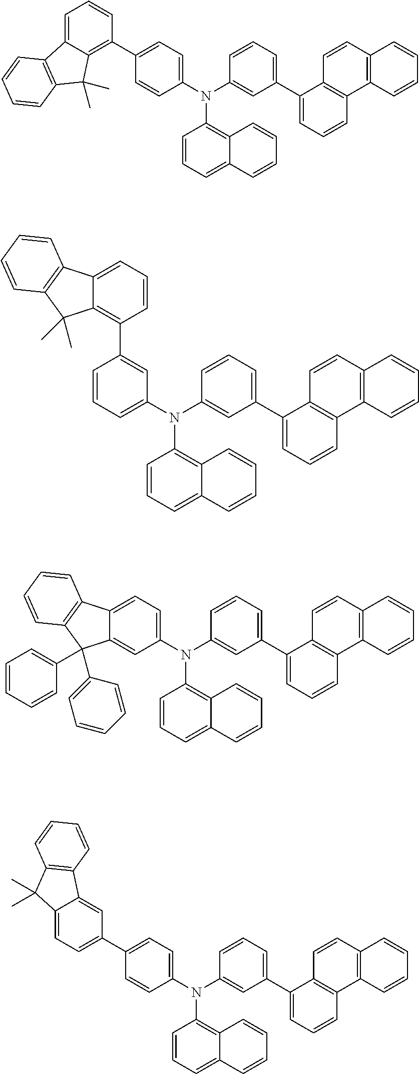 [SMILES: CC1(C)c2ccccc2-c2cccc(-c3ccc(N(c4cccc(-c5cccc6c5ccc5ccccc56)c4)c4cccc5ccccc45)cc3)c21 ; CC1(C)c2ccccc2-c2cccc(-c3cccc(N(c4cccc(-c5cccc6c5ccc5ccccc56)c4)c4cccc5ccccc45)c3)c21 ; c1ccc(C2(c3ccccc3)c3ccccc3-c3ccc(N(c4cccc(-c5cccc6c5ccc5ccccc56)c4)c4cccc5ccccc45)cc32)cc1 ; CC1(C)c2ccccc2-c2cc(-c3ccc(N(c4cccc(-c5cccc6c5ccc5ccccc56)c4)c4cccc5ccccc45)cc3)ccc21]

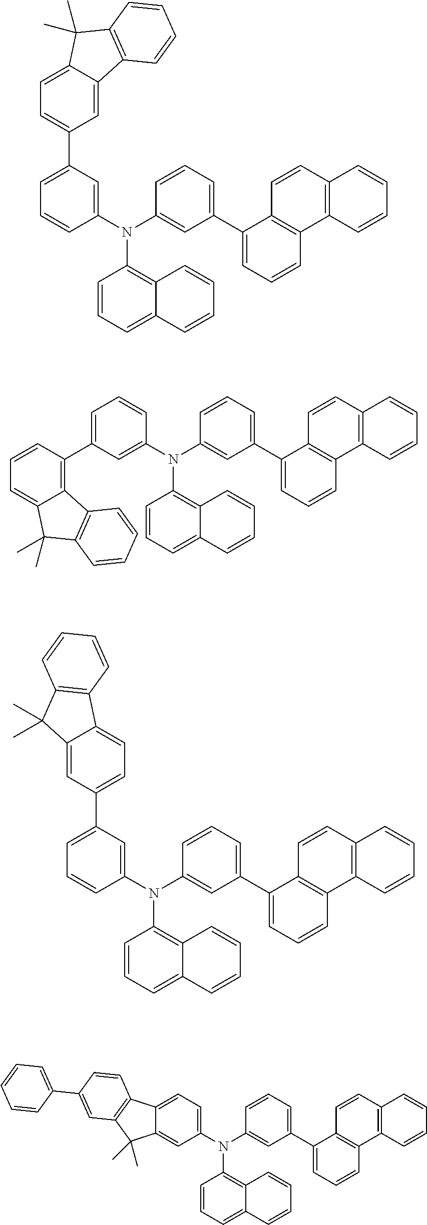 [SMILES: CC1(C)c2ccccc2-c2cc(-c3cccc(N(c4cccc(-c5cccc6c5ccc5ccccc56)c4)c4cccc5ccccc45)c3)ccc21 ; CC1(C)c2ccccc2-c2c(-c3cccc(N(c4cccc(-c5cccc6c5ccc5ccccc56)c4)c4cccc5ccccc45)c3)cccc21 ; CC1(C)c2ccccc2-c2ccc(-c3cccc(N(c4cccc(-c5cccc6c5ccc5ccccc56)c4)c4cccc5ccccc45)c3)cc21 ; CC1(C)c2cc(-c3ccccc3)ccc2-c2ccc(N(c3cccc(-c4cccc5c4ccc4ccccc45)c3)c3cccc4ccccc34)cc21]

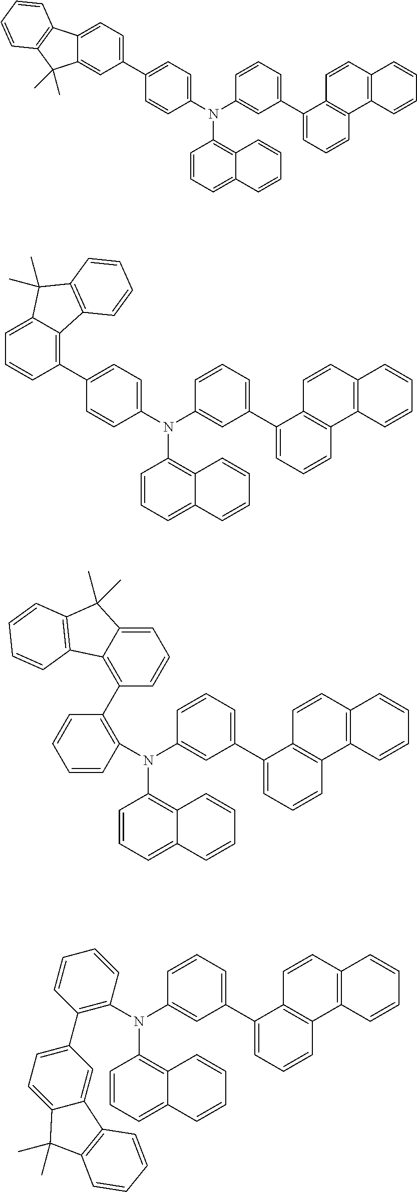 [SMILES: CC1(C)c2ccccc2-c2ccc(-c3ccc(N(c4cccc(-c5cccc6c5ccc5ccccc56)c4)c4cccc5ccccc45)cc3)cc21 ; CC1(C)c2ccccc2-c2c(-c3ccc(N(c4cccc(-c5cccc6c5ccc5ccccc56)c4)c4cccc5ccccc45)cc3)cccc21 ; CC1(C)c2ccccc2-c2c(-c3ccccc3N(c3cccc(-c4cccc5c4ccc4ccccc45)c3)c3cccc4ccccc34)cccc21 ; CC1(C)c2ccccc2-c2cc(-c3ccccc3N(c3cccc(-c4cccc5c4ccc4ccccc45)c3)c3cccc4ccccc34)ccc21]

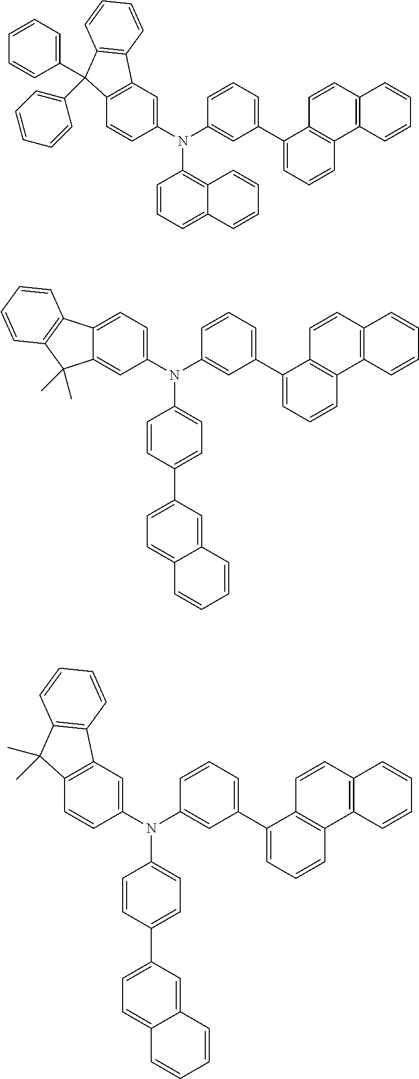 [SMILES: c1ccc(C2(c3ccccc3)c3ccccc3-c3cc(N(c4cccc(-c5cccc6c5ccc5ccccc56)c4)c4cccc5ccccc45)ccc32)cc1 ; CC1(C)c2ccccc2-c2ccc(N(c3ccc(-c4ccc5ccccc5c4)cc3)c3cccc(-c4cccc5c4ccc4ccccc45)c3)cc21 ; CC1(C)c2ccccc2-c2cc(N(c3ccc(-c4ccc5ccccc5c4)cc3)c3cccc(-c4cccc5c4ccc4ccccc45)c3)ccc21]

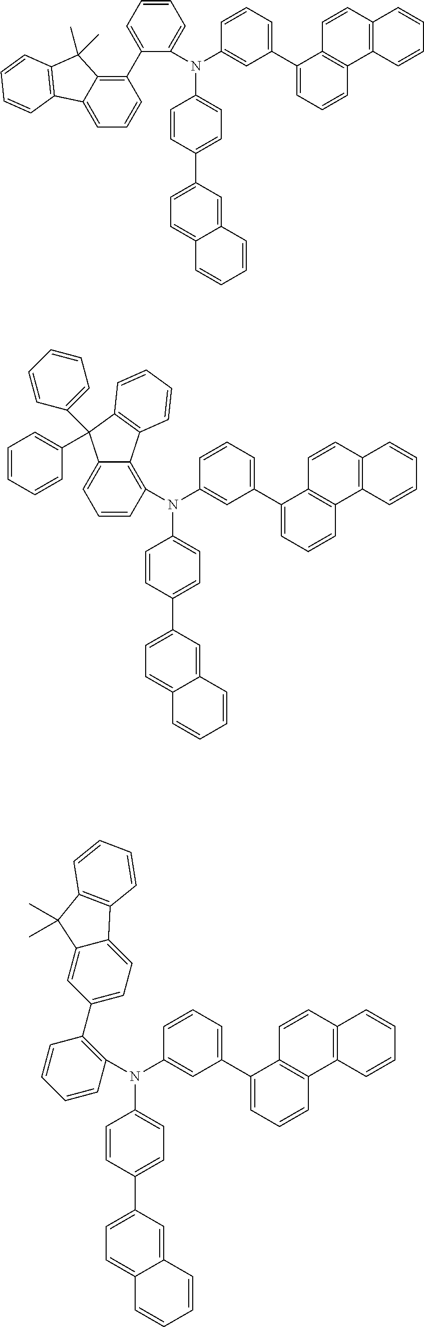 [SMILES: CC1(C)c2ccccc2-c2cccc(-c3ccccc3N(c3ccc(-c4ccc5ccccc5c4)cc3)c3cccc(-c4cccc5c4ccc4ccccc45)c3)c21 ; c1ccc(C2(c3ccccc3)c3ccccc3-c3c(N(c4ccc(-c5ccc6ccccc6c5)cc4)c4cccc(-c5cccc6c5ccc5ccccc56)c4)cccc32)cc1 ; CC1(C)c2ccccc2-c2ccc(-c3ccccc3N(c3ccc(-c4ccc5ccccc5c4)cc3)c3cccc(-c4cccc5c4ccc4ccccc45)c3)cc21]

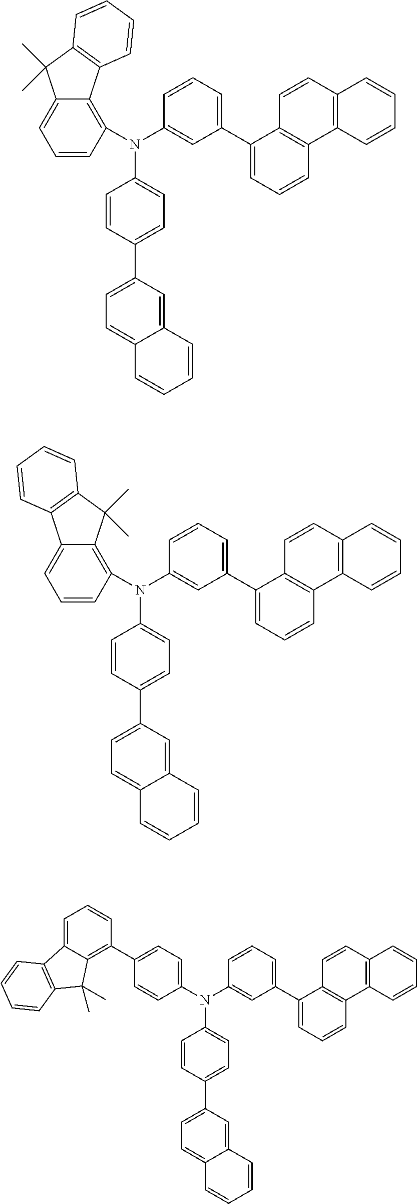 [SMILES: CC1(C)c2ccccc2-c2c(N(c3ccc(-c4ccc5ccccc5c4)cc3)c3cccc(-c4cccc5c4ccc4ccccc45)c3)cccc21 ; CC1(C)c2ccccc2-c2cccc(N(c3ccc(-c4ccc5ccccc5c4)cc3)c3cccc(-c4cccc5c4ccc4ccccc45)c3)c21 ; CC1(C)c2ccccc2-c2cccc(-c3ccc(N(c4ccc(-c5ccc6ccccc6c5)cc4)c4cccc(-c5cccc6c5ccc5ccccc56)c4)cc3)c21]

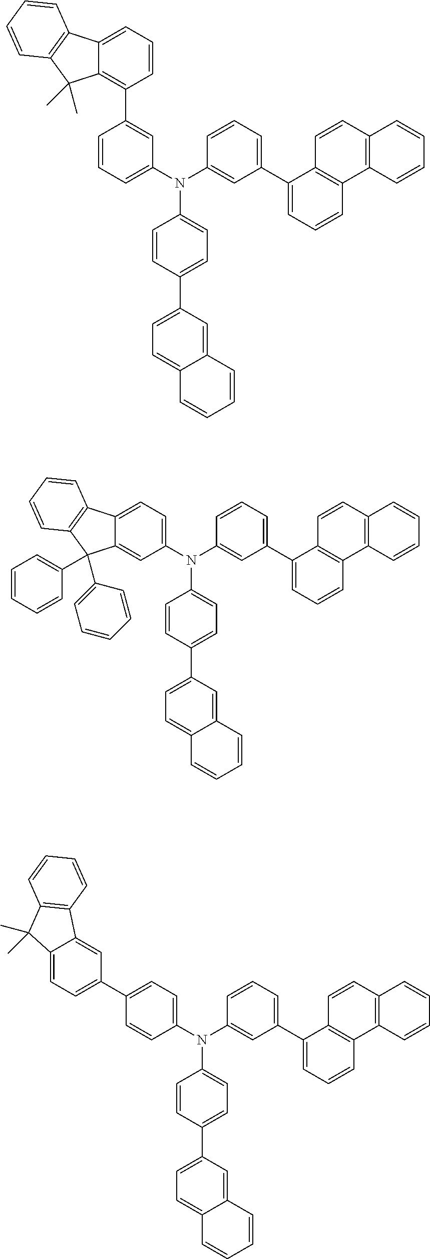 [SMILES: CC1(C)c2ccccc2-c2cccc(-c3cccc(N(c4ccc(-c5ccc6ccccc6c5)cc4)c4cccc(-c5cccc6c5ccc5ccccc56)c4)c3)c21 ; c1ccc(C2(c3ccccc3)c3ccccc3-c3ccc(N(c4ccc(-c5ccc6ccccc6c5)cc4)c4cccc(-c5cccc6c5ccc5ccccc56)c4)cc32)cc1 ; CC1(C)c2ccccc2-c2cc(-c3ccc(N(c4ccc(-c5ccc6ccccc6c5)cc4)c4cccc(-c5cccc6c5ccc5ccccc56)c4)cc3)ccc21]

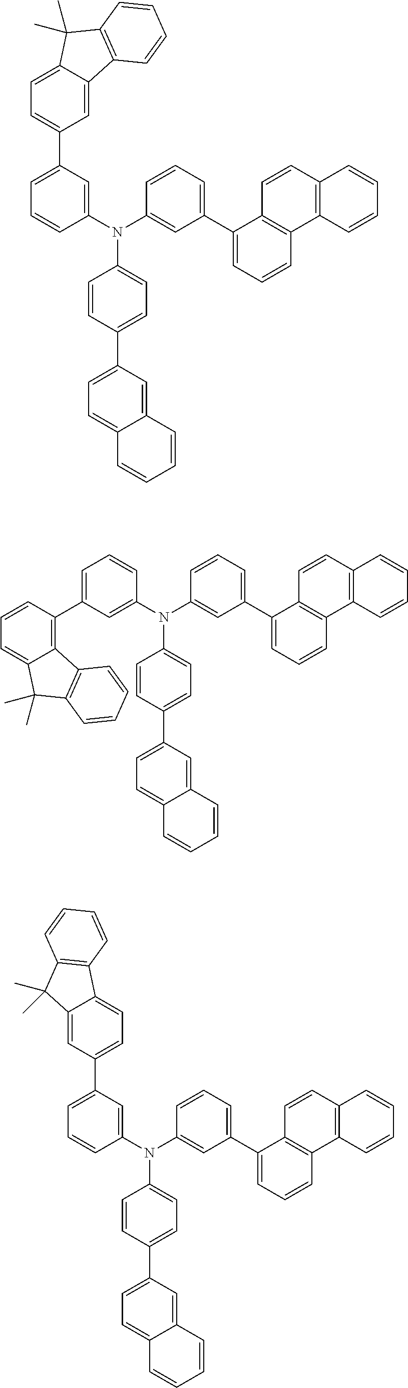 [SMILES: CC1(C)c2ccccc2-c2cc(-c3cccc(N(c4ccc(-c5ccc6ccccc6c5)cc4)c4cccc(-c5cccc6c5ccc5ccccc56)c4)c3)ccc21 ; CC1(C)c2ccccc2-c2c(-c3cccc(N(c4ccc(-c5ccc6ccccc6c5)cc4)c4cccc(-c5cccc6c5ccc5ccccc56)c4)c3)cccc21 ; CC1(C)c2ccccc2-c2ccc(-c3cccc(N(c4ccc(-c5ccc6ccccc6c5)cc4)c4cccc(-c5cccc6c5ccc5ccccc56)c4)c3)cc21]

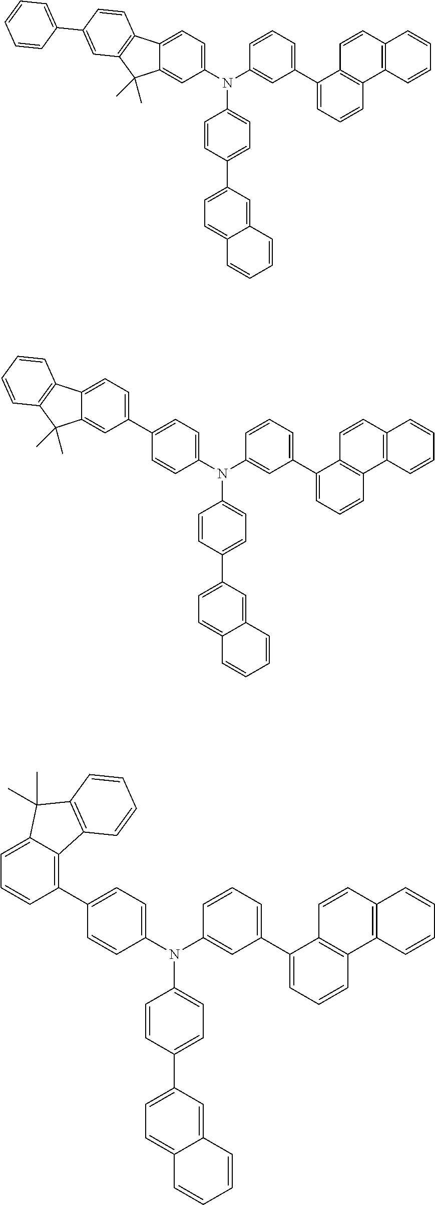 [SMILES: CC1(C)c2cc(-c3ccccc3)ccc2-c2ccc(N(c3ccc(-c4ccc5ccccc5c4)cc3)c3cccc(-c4cccc5c4ccc4ccccc45)c3)cc21 ; CC1(C)c2ccccc2-c2ccc(-c3ccc(N(c4ccc(-c5ccc6ccccc6c5)cc4)c4cccc(-c5cccc6c5ccc5ccccc56)c4)cc3)cc21 ; CC1(C)c2ccccc2-c2c(-c3ccc(N(c4ccc(-c5ccc6ccccc6c5)cc4)c4cccc(-c5cccc6c5ccc5ccccc56)c4)cc3)cccc21]

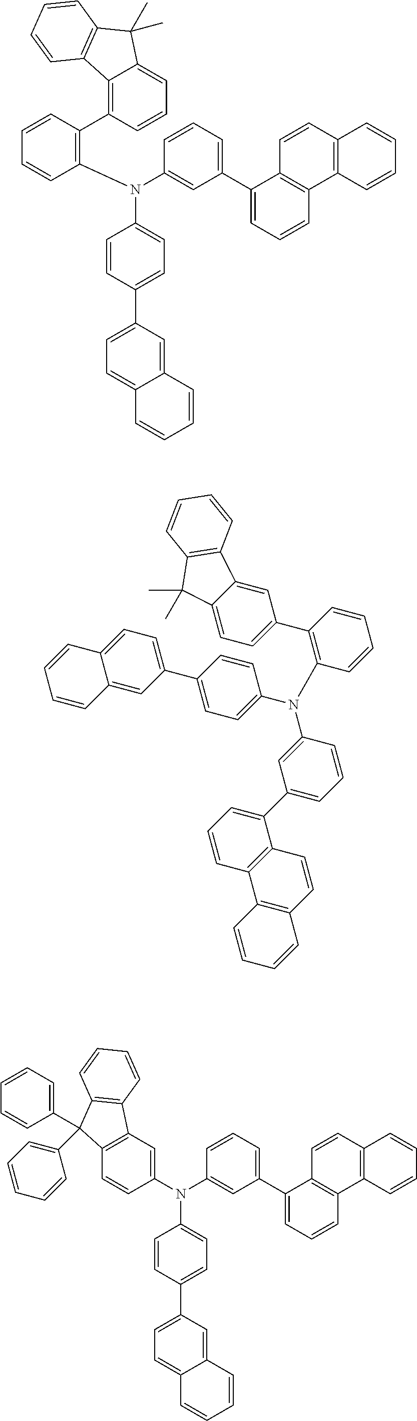 [SMILES: CC1(C)c2ccccc2-c2c(-c3ccccc3N(c3ccc(-c4ccc5ccccc5c4)cc3)c3cccc(-c4cccc5c4ccc4ccccc45)c3)cccc21 ; CC1(C)c2ccccc2-c2cc(-c3ccccc3N(c3ccc(-c4ccc5ccccc5c4)cc3)c3cccc(-c4cccc5c4ccc4ccccc45)c3)ccc21 ; c1ccc(C2(c3ccccc3)c3ccccc3-c3cc(N(c4ccc(-c5ccc6ccccc6c5)cc4)c4cccc(-c5cccc6c5ccc5ccccc56)c4)ccc32)cc1]

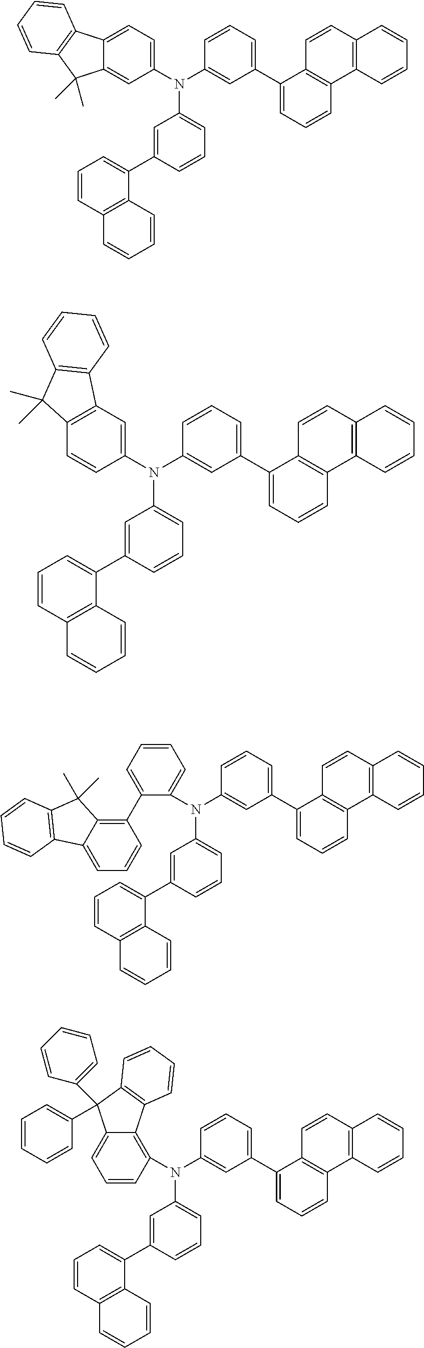 [SMILES: CC1(C)c2ccccc2-c2ccc(N(c3cccc(-c4cccc5ccccc45)c3)c3cccc(-c4cccc5c4ccc4ccccc45)c3)cc21 ; CC1(C)c2ccccc2-c2cc(N(c3cccc(-c4cccc5ccccc45)c3)c3cccc(-c4cccc5c4ccc4ccccc45)c3)ccc21 ; CC1(C)c2ccccc2-c2cccc(-c3ccccc3N(c3cccc(-c4cccc5ccccc45)c3)c3cccc(-c4cccc5c4ccc4ccccc45)c3)c21 ; c1ccc(C2(c3ccccc3)c3ccccc3-c3c(N(c4cccc(-c5cccc6ccccc56)c4)c4cccc(-c5cccc6c5ccc5ccccc56)c4)cccc32)cc1]

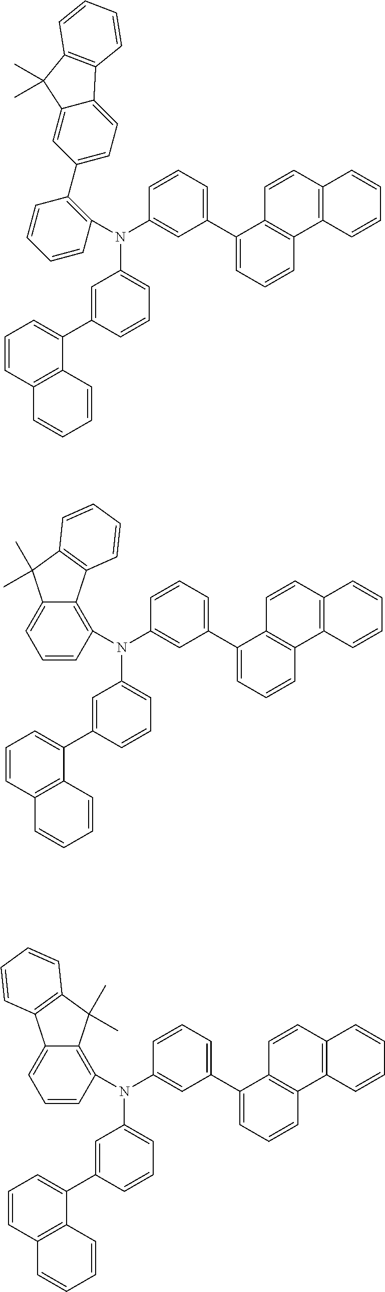 [SMILES: CC1(C)c2ccccc2-c2ccc(-c3ccccc3N(c3cccc(-c4cccc5ccccc45)c3)c3cccc(-c4cccc5c4ccc4ccccc45)c3)cc21 ; CC1(C)c2ccccc2-c2c(N(c3cccc(-c4cccc5ccccc45)c3)c3cccc(-c4cccc5c4ccc4ccccc45)c3)cccc21 ; CC1(C)c2ccccc2-c2cccc(N(c3cccc(-c4cccc5ccccc45)c3)c3cccc(-c4cccc5c4ccc4ccccc45)c3)c21]

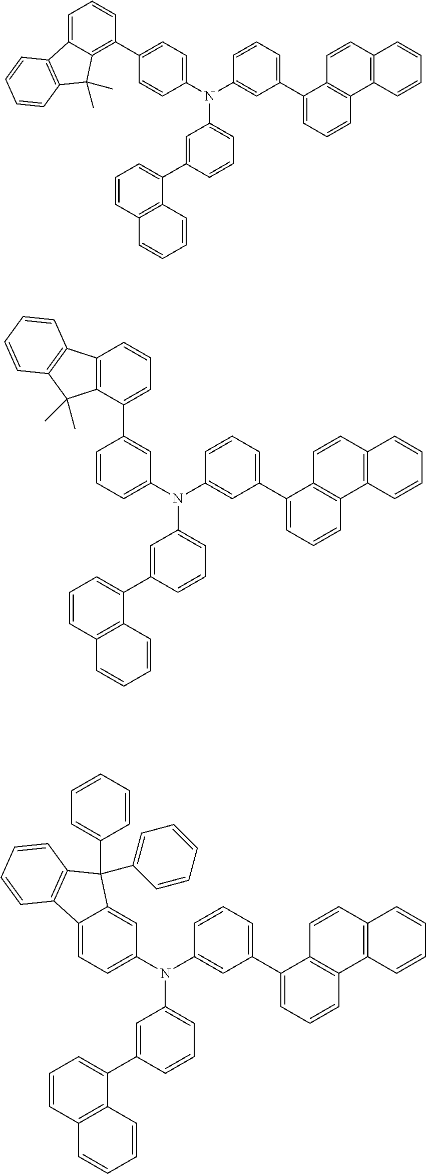 [SMILES: CC1(C)c2ccccc2-c2cccc(-c3ccc(N(c4cccc(-c5cccc6ccccc56)c4)c4cccc(-c5cccc6c5ccc5ccccc56)c4)cc3)c21 ; CC1(C)c2ccccc2-c2cccc(-c3cccc(N(c4cccc(-c5cccc6ccccc56)c4)c4cccc(-c5cccc6c5ccc5ccccc56)c4)c3)c21 ; c1ccc(C2(c3ccccc3)c3ccccc3-c3ccc(N(c4cccc(-c5cccc6ccccc56)c4)c4cccc(-c5cccc6c5ccc5ccccc56)c4)cc32)cc1]

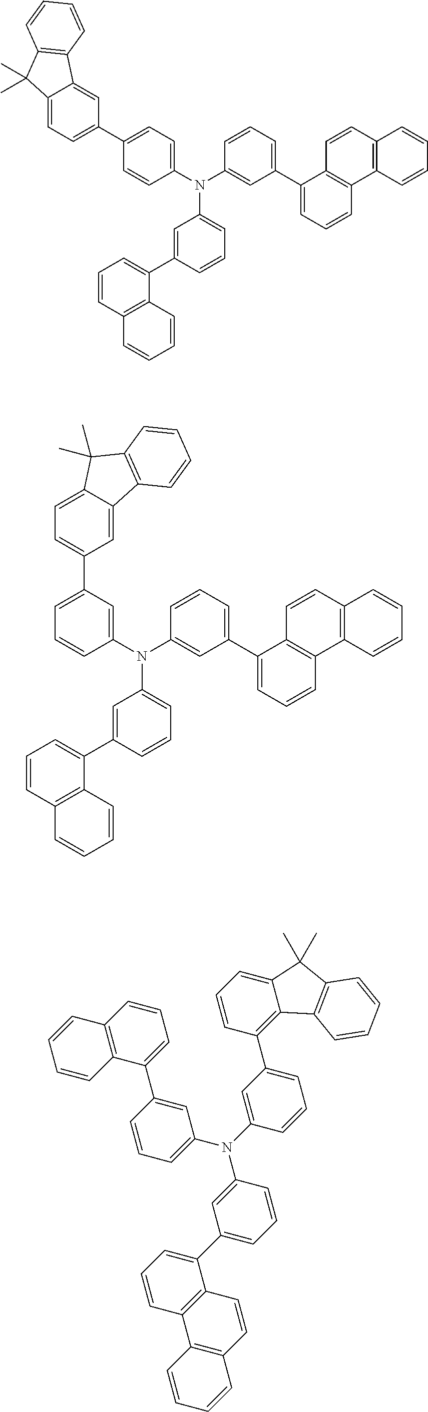 [SMILES: CC1(C)c2ccccc2-c2cc(-c3ccc(N(c4cccc(-c5cccc6ccccc56)c4)c4cccc(-c5cccc6c5ccc5ccccc56)c4)cc3)ccc21 ; CC1(C)c2ccccc2-c2cc(-c3cccc(N(c4cccc(-c5cccc6ccccc56)c4)c4cccc(-c5cccc6c5ccc5ccccc56)c4)c3)ccc21 ; CC1(C)c2ccccc2-c2c(-c3cccc(N(c4cccc(-c5cccc6ccccc56)c4)c4cccc(-c5cccc6c5ccc5ccccc56)c4)c3)cccc21]

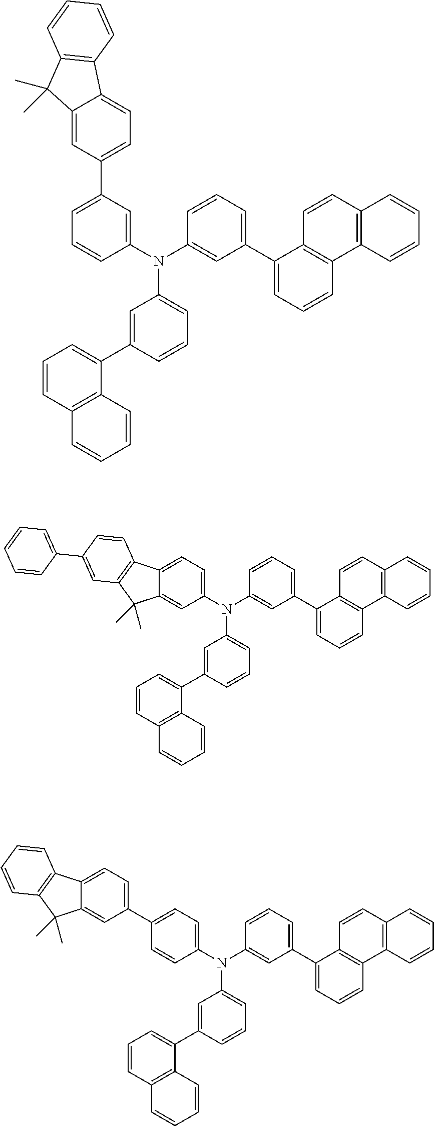 [SMILES: CC1(C)c2ccccc2-c2ccc(-c3cccc(N(c4cccc(-c5cccc6ccccc56)c4)c4cccc(-c5cccc6c5ccc5ccccc56)c4)c3)cc21 ; CC1(C)c2cc(-c3ccccc3)ccc2-c2ccc(N(c3cccc(-c4cccc5ccccc45)c3)c3cccc(-c4cccc5c4ccc4ccccc45)c3)cc21 ; CC1(C)c2ccccc2-c2ccc(-c3ccc(N(c4cccc(-c5cccc6ccccc56)c4)c4cccc(-c5cccc6c5ccc5ccccc56)c4)cc3)cc21]

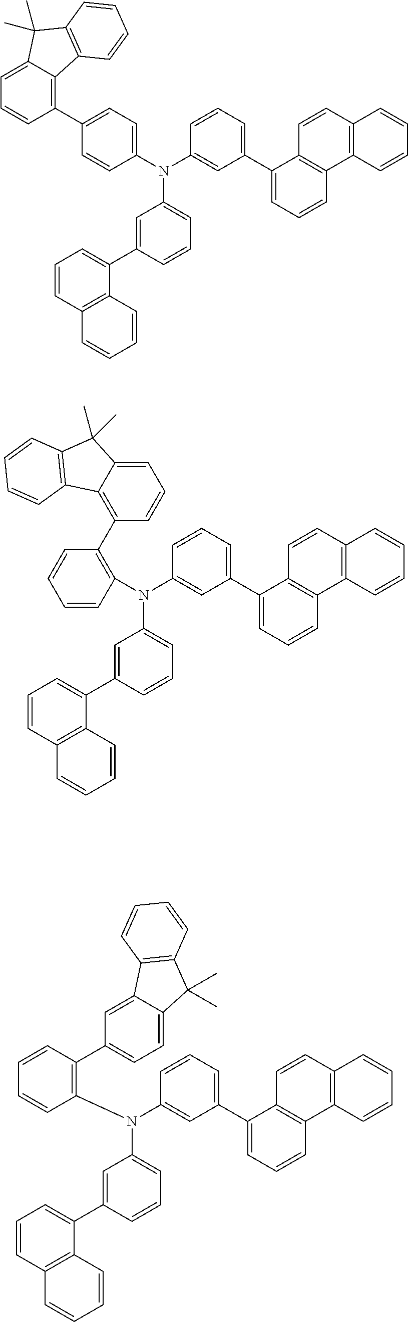 [SMILES: CC1(C)c2ccccc2-c2c(-c3ccc(N(c4cccc(-c5cccc6ccccc56)c4)c4cccc(-c5cccc6c5ccc5ccccc56)c4)cc3)cccc21 ; CC1(C)c2ccccc2-c2c(-c3ccccc3N(c3cccc(-c4cccc5ccccc45)c3)c3cccc(-c4cccc5c4ccc4ccccc45)c3)cccc21 ; CC1(C)c2ccccc2-c2cc(-c3ccccc3N(c3cccc(-c4cccc5ccccc45)c3)c3cccc(-c4cccc5c4ccc4ccccc45)c3)ccc21]

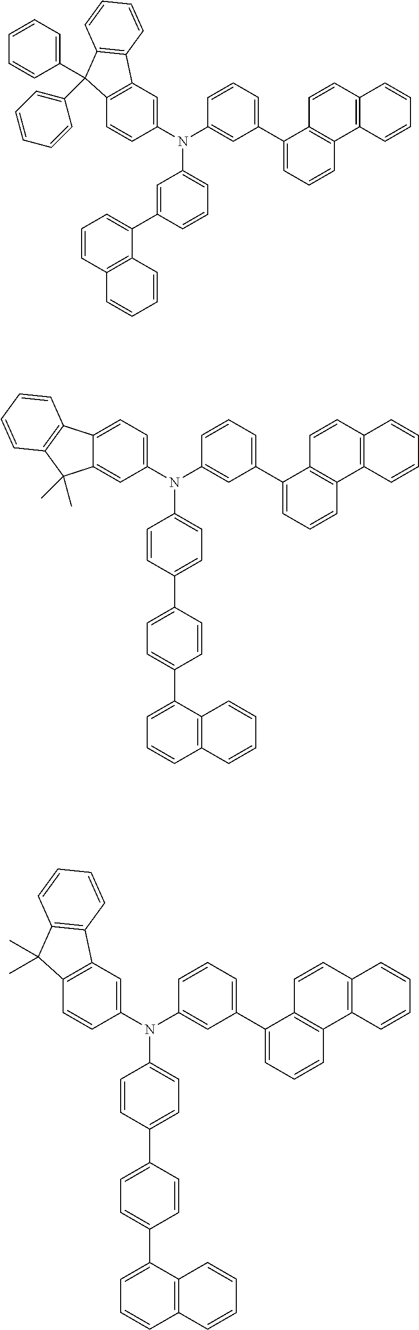 [SMILES: c1ccc(C2(c3ccccc3)c3ccccc3-c3cc(N(c4cccc(-c5cccc6ccccc56)c4)c4cccc(-c5cccc6c5ccc5ccccc56)c4)ccc32)cc1 ; CC1(C)c2ccccc2-c2ccc(N(c3ccc(-c4ccc(-c5cccc6ccccc56)cc4)cc3)c3cccc(-c4cccc5c4ccc4ccccc45)c3)cc21 ; CC1(C)c2ccccc2-c2cc(N(c3ccc(-c4ccc(-c5cccc6ccccc56)cc4)cc3)c3cccc(-c4cccc5c4ccc4ccccc45)c3)ccc21]

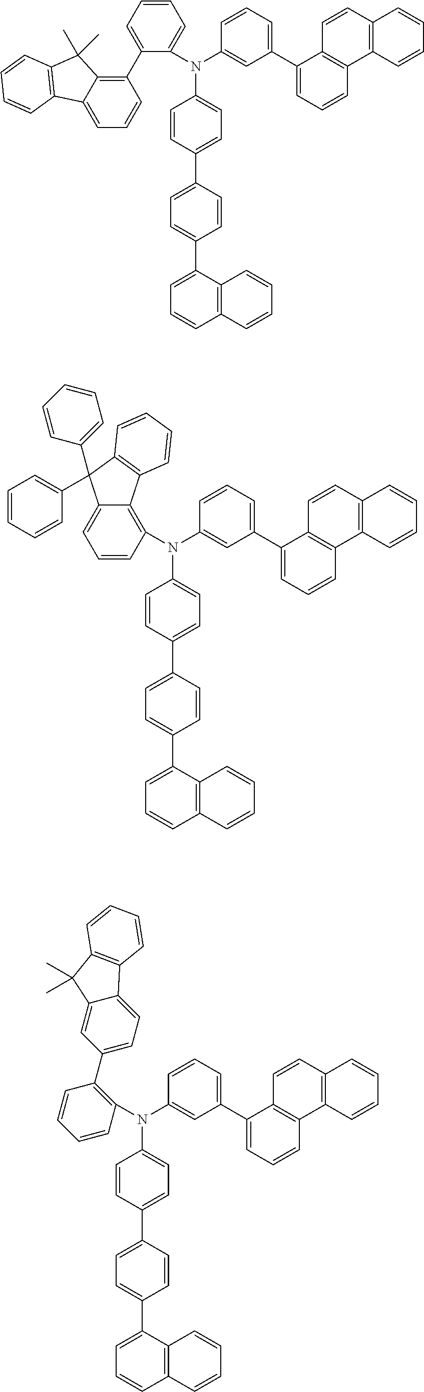 [SMILES: CC1(C)c2ccccc2-c2cccc(-c3ccccc3N(c3ccc(-c4ccc(-c5cccc6ccccc56)cc4)cc3)c3cccc(-c4cccc5c4ccc4ccccc45)c3)c21 ; c1ccc(C2(c3ccccc3)c3ccccc3-c3c(N(c4ccc(-c5ccc(-c6cccc7ccccc67)cc5)cc4)c4cccc(-c5cccc6c5ccc5ccccc56)c4)cccc32)cc1 ; CC1(C)c2ccccc2-c2ccc(-c3ccccc3N(c3ccc(-c4ccc(-c5cccc6ccccc56)cc4)cc3)c3cccc(-c4cccc5c4ccc4ccccc45)c3)cc21]

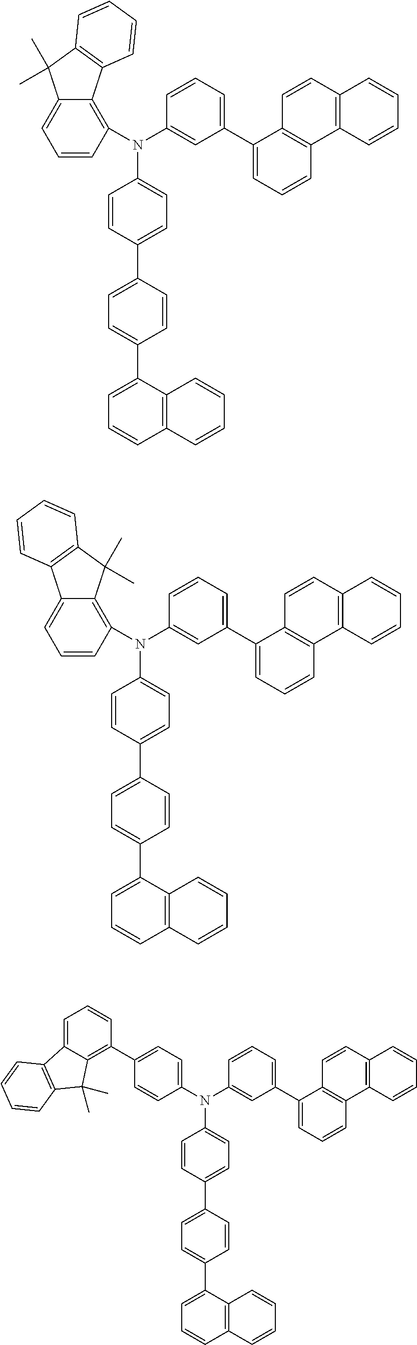 [SMILES: CC1(C)c2ccccc2-c2c(N(c3ccc(-c4ccc(-c5cccc6ccccc56)cc4)cc3)c3cccc(-c4cccc5c4ccc4ccccc45)c3)cccc21 ; CC1(C)c2ccccc2-c2cccc(N(c3ccc(-c4ccc(-c5cccc6ccccc56)cc4)cc3)c3cccc(-c4cccc5c4ccc4ccccc45)c3)c21 ; CC1(C)c2ccccc2-c2cccc(-c3ccc(N(c4ccc(-c5ccc(-c6cccc7ccccc67)cc5)cc4)c4cccc(-c5cccc6c5ccc5ccccc56)c4)cc3)c21]

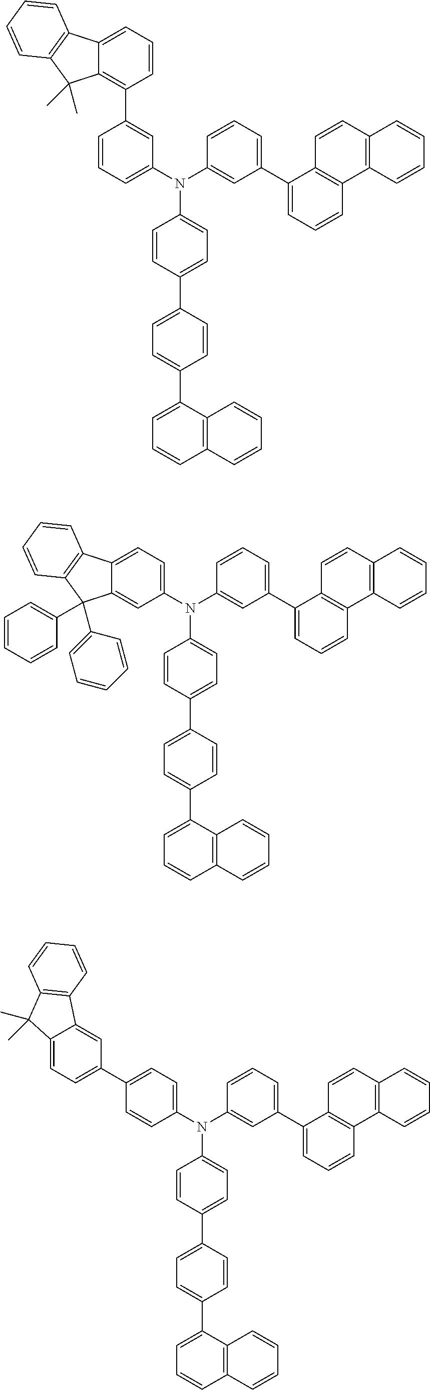 [SMILES: CC1(C)c2ccccc2-c2cccc(-c3cccc(N(c4ccc(-c5ccc(-c6cccc7ccccc67)cc5)cc4)c4cccc(-c5cccc6c5ccc5ccccc56)c4)c3)c21 ; c1ccc(C2(c3ccccc3)c3ccccc3-c3ccc(N(c4ccc(-c5ccc(-c6cccc7ccccc67)cc5)cc4)c4cccc(-c5cccc6c5ccc5ccccc56)c4)cc32)cc1 ; CC1(C)c2ccccc2-c2cc(-c3ccc(N(c4ccc(-c5ccc(-c6cccc7ccccc67)cc5)cc4)c4cccc(-c5cccc6c5ccc5ccccc56)c4)cc3)ccc21]

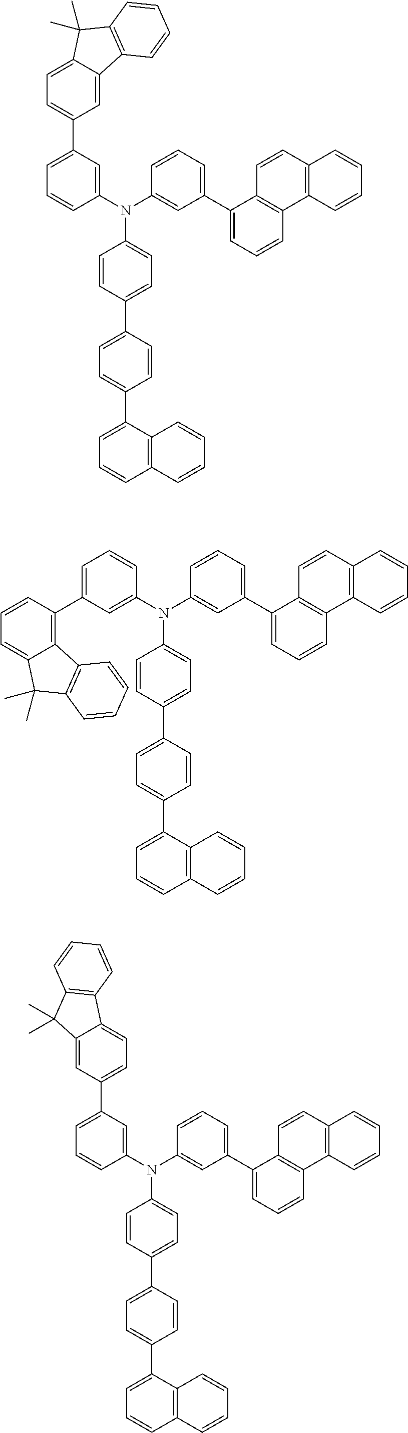 [SMILES: CC1(C)c2ccccc2-c2cc(-c3cccc(N(c4ccc(-c5ccc(-c6cccc7ccccc67)cc5)cc4)c4cccc(-c5cccc6c5ccc5ccccc56)c4)c3)ccc21 ; CC1(C)c2ccccc2-c2c(-c3cccc(N(c4ccc(-c5ccc(-c6cccc7ccccc67)cc5)cc4)c4cccc(-c5cccc6c5ccc5ccccc56)c4)c3)cccc21 ; CC1(C)c2ccccc2-c2ccc(-c3cccc(N(c4ccc(-c5ccc(-c6cccc7ccccc67)cc5)cc4)c4cccc(-c5cccc6c5ccc5ccccc56)c4)c3)cc21]

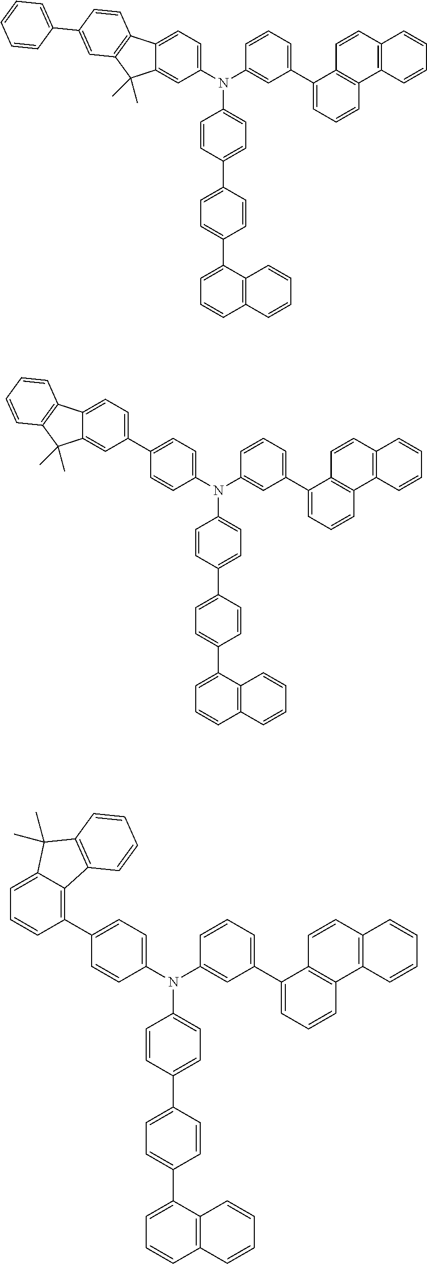 [SMILES: CC1(C)c2cc(-c3ccccc3)ccc2-c2ccc(N(c3ccc(-c4ccc(-c5cccc6ccccc56)cc4)cc3)c3cccc(-c4cccc5c4ccc4ccccc45)c3)cc21 ; CC1(C)c2ccccc2-c2ccc(-c3ccc(N(c4ccc(-c5ccc(-c6cccc7ccccc67)cc5)cc4)c4cccc(-c5cccc6c5ccc5ccccc56)c4)cc3)cc21 ; CC1(C)c2ccccc2-c2c(-c3ccc(N(c4ccc(-c5ccc(-c6cccc7ccccc67)cc5)cc4)c4cccc(-c5cccc6c5ccc5ccccc56)c4)cc3)cccc21]

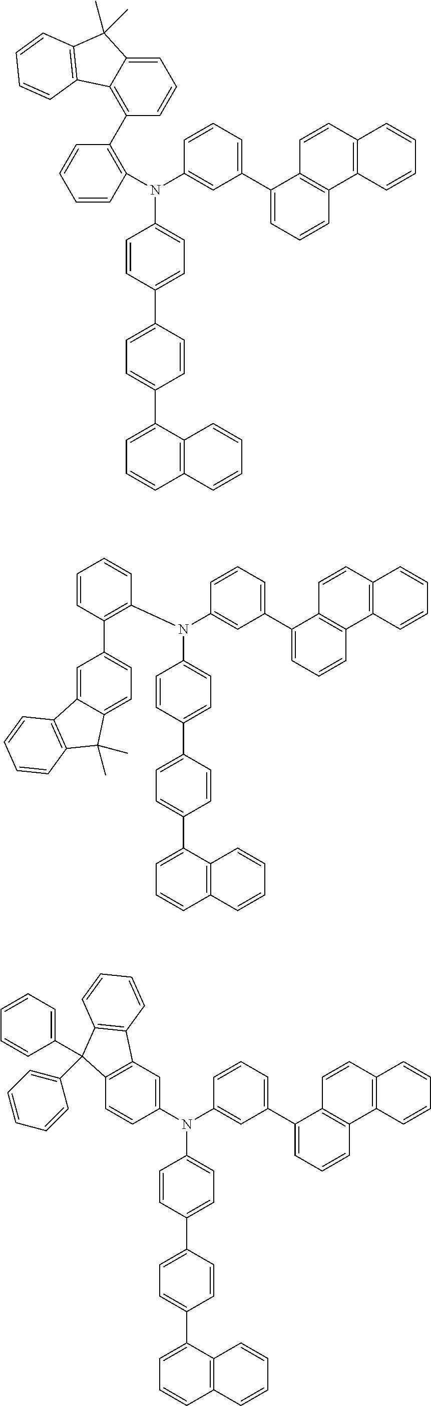 [SMILES: CC1(C)c2ccccc2-c2c(-c3ccccc3N(c3ccc(-c4ccc(-c5cccc6ccccc56)cc4)cc3)c3cccc(-c4cccc5c4ccc4ccccc45)c3)cccc21 ; CC1(C)c2ccccc2-c2cc(-c3ccccc3N(c3ccc(-c4ccc(-c5cccc6ccccc56)cc4)cc3)c3cccc(-c4cccc5c4ccc4ccccc45)c3)ccc21 ; c1ccc(C2(c3ccccc3)c3ccccc3-c3cc(N(c4ccc(-c5ccc(-c6cccc7ccccc67)cc5)cc4)c4cccc(-c5cccc6c5ccc5ccccc56)c4)ccc32)cc1]

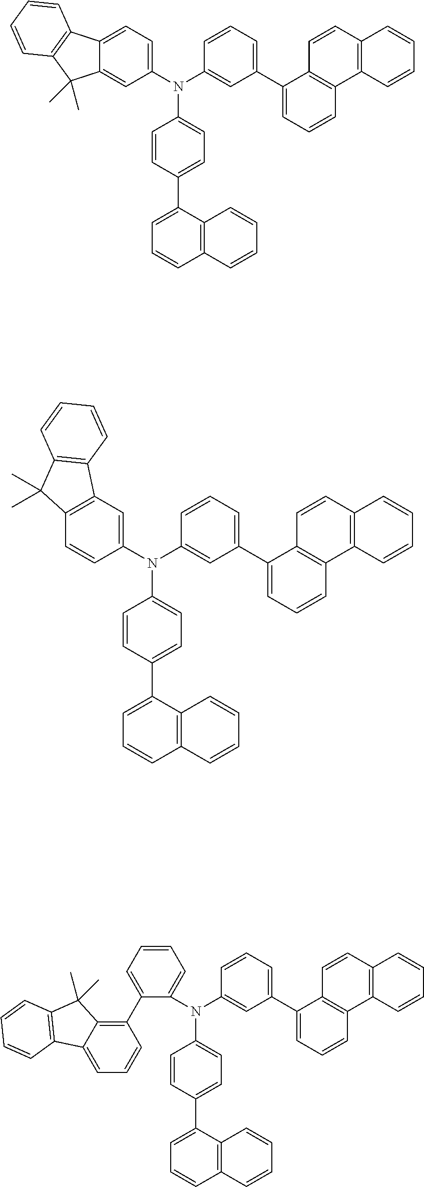 [SMILES: CC1(C)c2ccccc2-c2ccc(N(c3ccc(-c4cccc5ccccc45)cc3)c3cccc(-c4cccc5c4ccc4ccccc45)c3)cc21 ; CC1(C)c2ccccc2-c2cc(N(c3ccc(-c4cccc5ccccc45)cc3)c3cccc(-c4cccc5c4ccc4ccccc45)c3)ccc21 ; CC1(C)c2ccccc2-c2cccc(-c3ccccc3N(c3ccc(-c4cccc5ccccc45)cc3)c3cccc(-c4cccc5c4ccc4ccccc45)c3)c21]

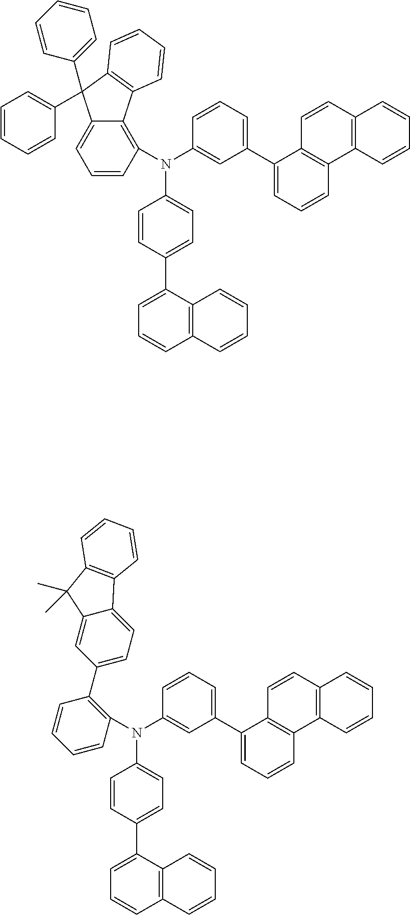 [SMILES: c1ccc(C2(c3ccccc3)c3ccccc3-c3c(N(c4ccc(-c5cccc6ccccc56)cc4)c4cccc(-c5cccc6c5ccc5ccccc56)c4)cccc32)cc1 ; CC1(C)c2ccccc2-c2ccc(-c3ccccc3N(c3ccc(-c4cccc5ccccc45)cc3)c3cccc(-c4cccc5c4ccc4ccccc45)c3)cc21]

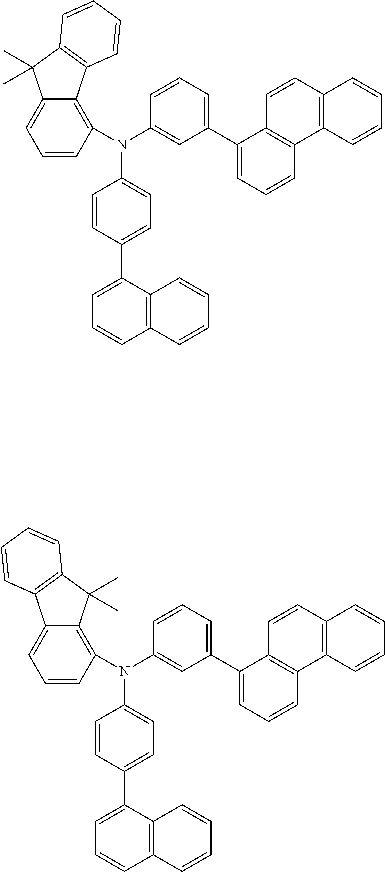 [SMILES: CC1(C)c2ccccc2-c2c(N(c3ccc(-c4cccc5ccccc45)cc3)c3cccc(-c4cccc5c4ccc4ccccc45)c3)cccc21 ; CC1(C)c2ccccc2-c2cccc(N(c3ccc(-c4cccc5ccccc45)cc3)c3cccc(-c4cccc5c4ccc4ccccc45)c3)c21]

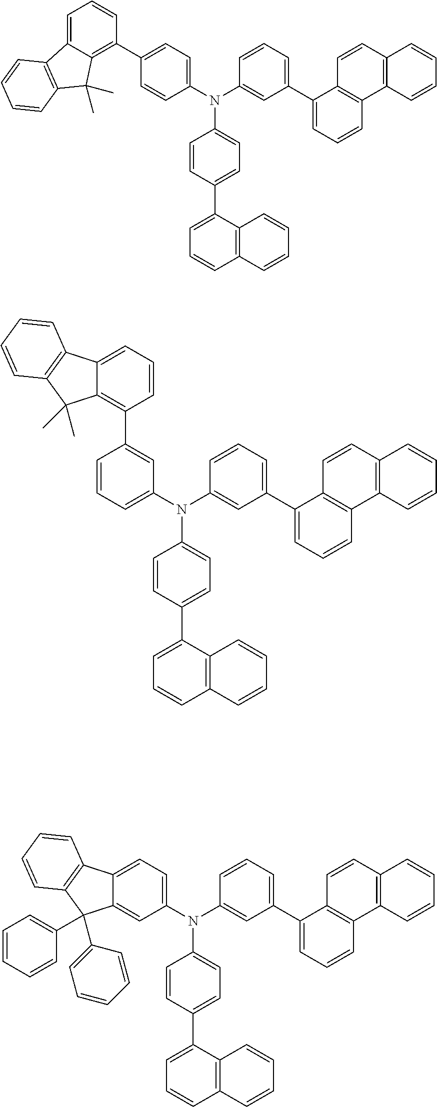 [SMILES: CC1(C)c2ccccc2-c2cccc(-c3ccc(N(c4ccc(-c5cccc6ccccc56)cc4)c4cccc(-c5cccc6c5ccc5ccccc56)c4)cc3)c21 ; CC1(C)c2ccccc2-c2cccc(-c3cccc(N(c4ccc(-c5cccc6ccccc56)cc4)c4cccc(-c5cccc6c5ccc5ccccc56)c4)c3)c21 ; c1ccc(C2(c3ccccc3)c3ccccc3-c3ccc(N(c4ccc(-c5cccc6ccccc56)cc4)c4cccc(-c5cccc6c5ccc5ccccc56)c4)cc32)cc1]

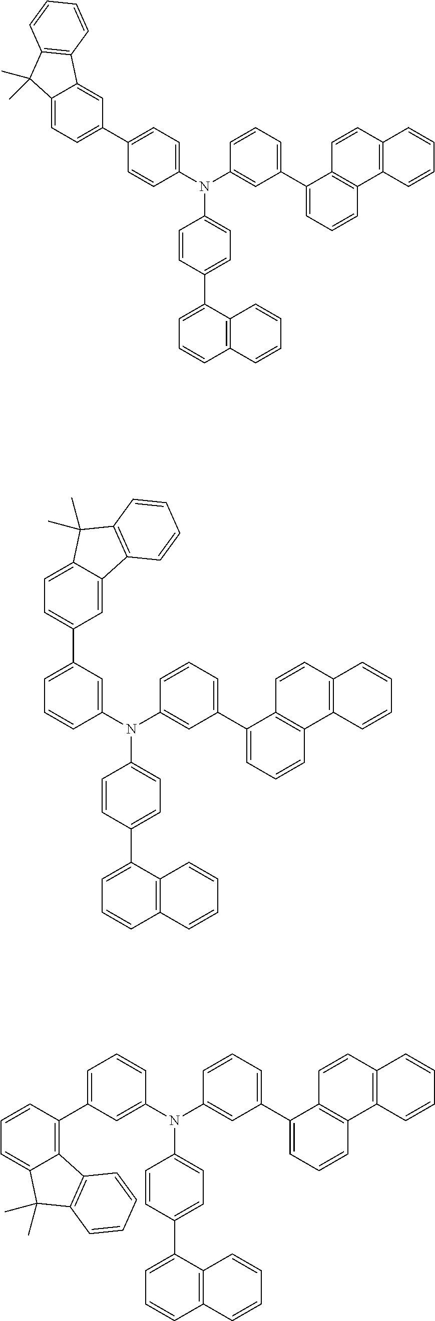 [SMILES: CC1(C)c2ccccc2-c2cc(-c3ccc(N(c4ccc(-c5cccc6ccccc56)cc4)c4cccc(-c5cccc6c5ccc5ccccc56)c4)cc3)ccc21 ; CC1(C)c2ccccc2-c2cc(-c3cccc(N(c4ccc(-c5cccc6ccccc56)cc4)c4cccc(-c5cccc6c5ccc5ccccc56)c4)c3)ccc21 ; CC1(C)c2ccccc2-c2c(-c3cccc(N(c4ccc(-c5cccc6ccccc56)cc4)c4cccc(-c5cccc6c5ccc5ccccc56)c4)c3)cccc21]

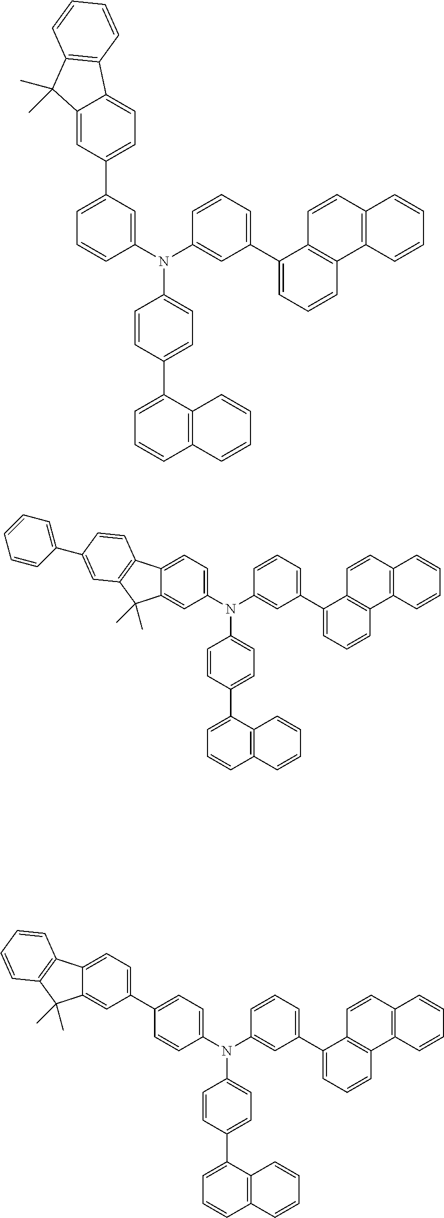 [SMILES: CC1(C)c2ccccc2-c2ccc(-c3cccc(N(c4ccc(-c5cccc6ccccc56)cc4)c4cccc(-c5cccc6c5ccc5ccccc56)c4)c3)cc21 ; CC1(C)c2cc(-c3ccccc3)ccc2-c2ccc(N(c3ccc(-c4cccc5ccccc45)cc3)c3cccc(-c4cccc5c4ccc4ccccc45)c3)cc21 ; CC1(C)c2ccccc2-c2ccc(-c3ccc(N(c4ccc(-c5cccc6ccccc56)cc4)c4cccc(-c5cccc6c5ccc5ccccc56)c4)cc3)cc21]

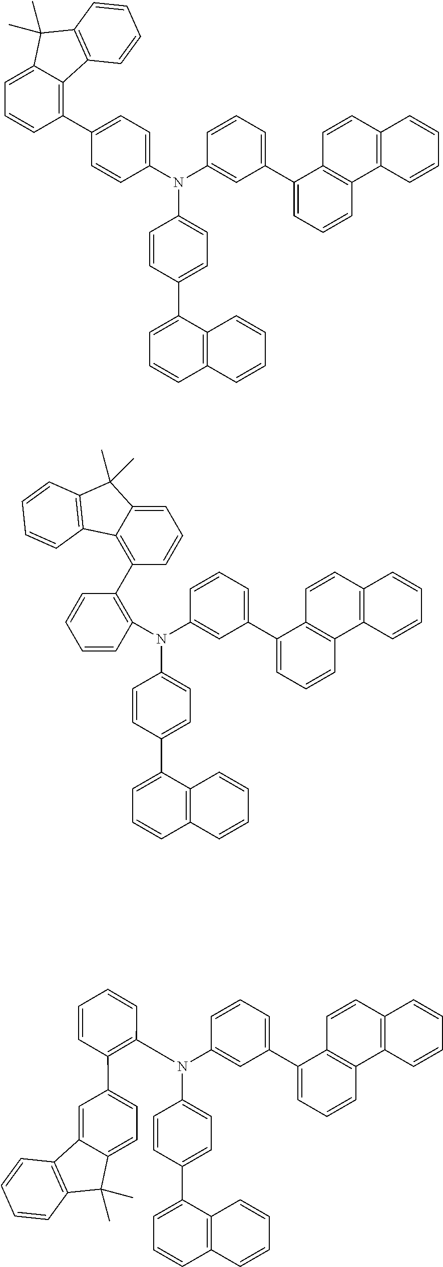 [SMILES: CC1(C)c2ccccc2-c2c(-c3ccc(N(c4ccc(-c5cccc6ccccc56)cc4)c4cccc(-c5cccc6c5ccc5ccccc56)c4)cc3)cccc21 ; CC1(C)c2ccccc2-c2c(-c3ccccc3N(c3ccc(-c4cccc5ccccc45)cc3)c3cccc(-c4cccc5c4ccc4ccccc45)c3)cccc21 ; CC1(C)c2ccccc2-c2cc(-c3ccccc3N(c3ccc(-c4cccc5ccccc45)cc3)c3cccc(-c4cccc5c4ccc4ccccc45)c3)ccc21]

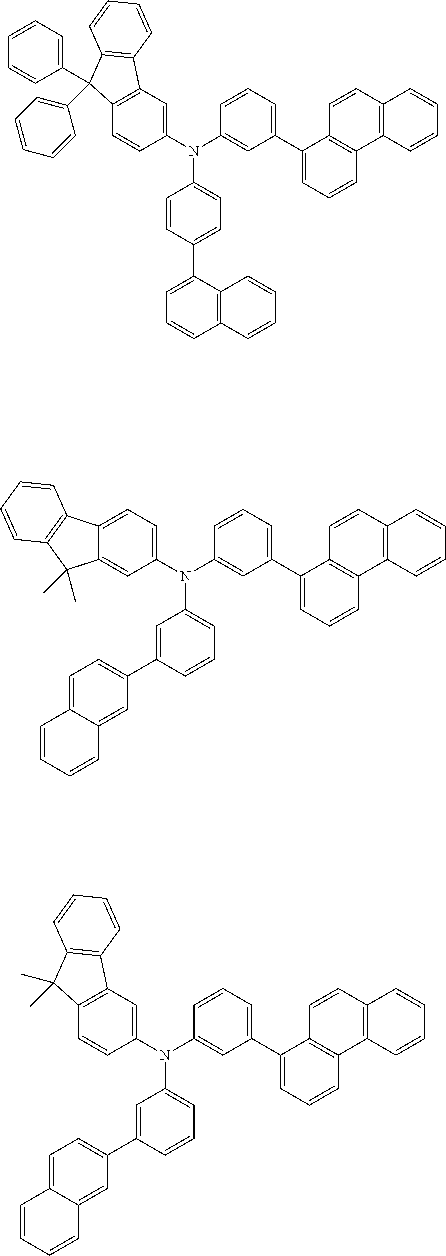 [SMILES: c1ccc(C2(c3ccccc3)c3ccccc3-c3cc(N(c4ccc(-c5cccc6ccccc56)cc4)c4cccc(-c5cccc6c5ccc5ccccc56)c4)ccc32)cc1 ; CC1(C)c2ccccc2-c2ccc(N(c3cccc(-c4ccc5ccccc5c4)c3)c3cccc(-c4cccc5c4ccc4ccccc45)c3)cc21 ; CC1(C)c2ccccc2-c2cc(N(c3cccc(-c4ccc5ccccc5c4)c3)c3cccc(-c4cccc5c4ccc4ccccc45)c3)ccc21]

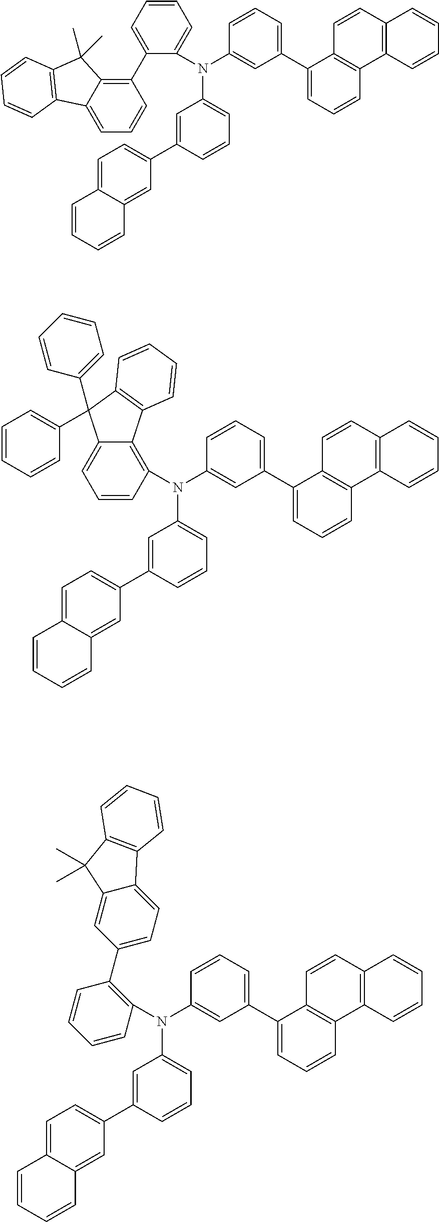 [SMILES: CC1(C)c2ccccc2-c2cccc(-c3ccccc3N(c3cccc(-c4ccc5ccccc5c4)c3)c3cccc(-c4cccc5c4ccc4ccccc45)c3)c21 ; c1ccc(C2(c3ccccc3)c3ccccc3-c3c(N(c4cccc(-c5ccc6ccccc6c5)c4)c4cccc(-c5cccc6c5ccc5ccccc56)c4)cccc32)cc1 ; CC1(C)c2ccccc2-c2ccc(-c3ccccc3N(c3cccc(-c4ccc5ccccc5c4)c3)c3cccc(-c4cccc5c4ccc4ccccc45)c3)cc21]

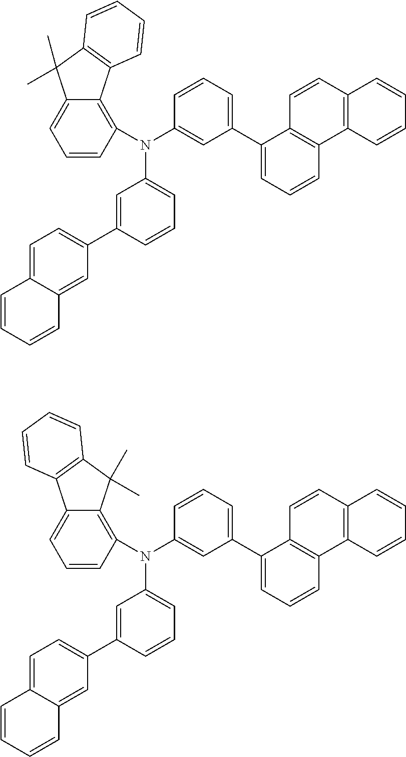 [SMILES: CC1(C)c2ccccc2-c2c(N(c3cccc(-c4ccc5ccccc5c4)c3)c3cccc(-c4cccc5c4ccc4ccccc45)c3)cccc21 ; CC1(C)c2ccccc2-c2cccc(N(c3cccc(-c4ccc5ccccc5c4)c3)c3cccc(-c4cccc5c4ccc4ccccc45)c3)c21]

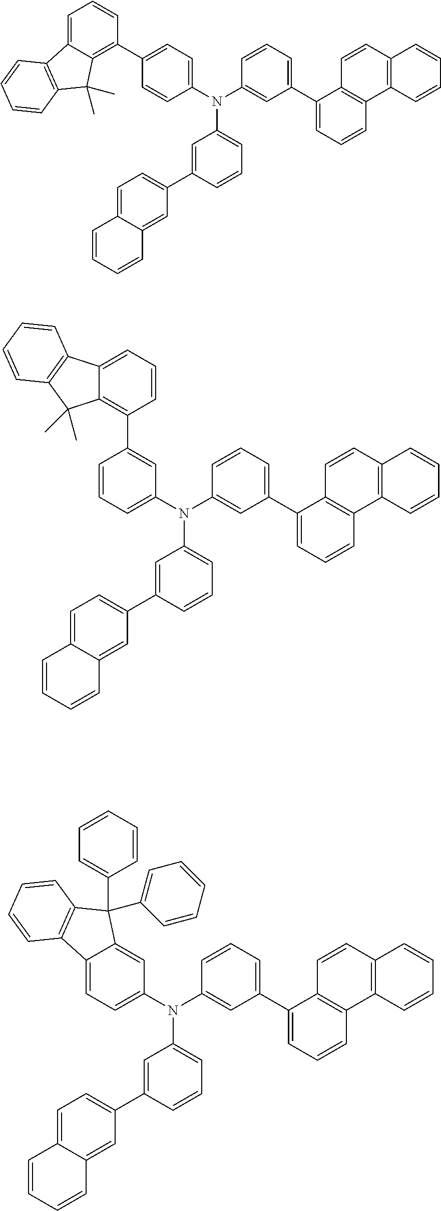 [SMILES: CC1(C)c2ccccc2-c2cccc(-c3ccc(N(c4cccc(-c5ccc6ccccc6c5)c4)c4cccc(-c5cccc6c5ccc5ccccc56)c4)cc3)c21 ; CC1(C)c2ccccc2-c2cccc(-c3cccc(N(c4cccc(-c5ccc6ccccc6c5)c4)c4cccc(-c5cccc6c5ccc5ccccc56)c4)c3)c21 ; c1ccc(C2(c3ccccc3)c3ccccc3-c3ccc(N(c4cccc(-c5ccc6ccccc6c5)c4)c4cccc(-c5cccc6c5ccc5ccccc56)c4)cc32)cc1]

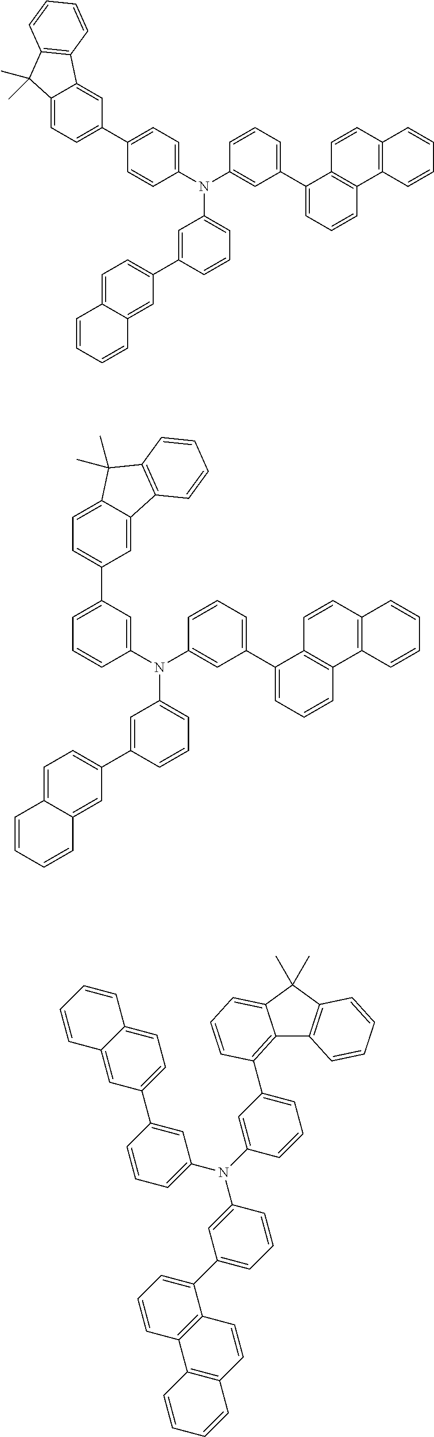 [SMILES: CC1(C)c2ccccc2-c2cc(-c3ccc(N(c4cccc(-c5ccc6ccccc6c5)c4)c4cccc(-c5cccc6c5ccc5ccccc56)c4)cc3)ccc21 ; CC1(C)c2ccccc2-c2cc(-c3cccc(N(c4cccc(-c5ccc6ccccc6c5)c4)c4cccc(-c5cccc6c5ccc5ccccc56)c4)c3)ccc21 ; CC1(C)c2ccccc2-c2c(-c3cccc(N(c4cccc(-c5ccc6ccccc6c5)c4)c4cccc(-c5cccc6c5ccc5ccccc56)c4)c3)cccc21]

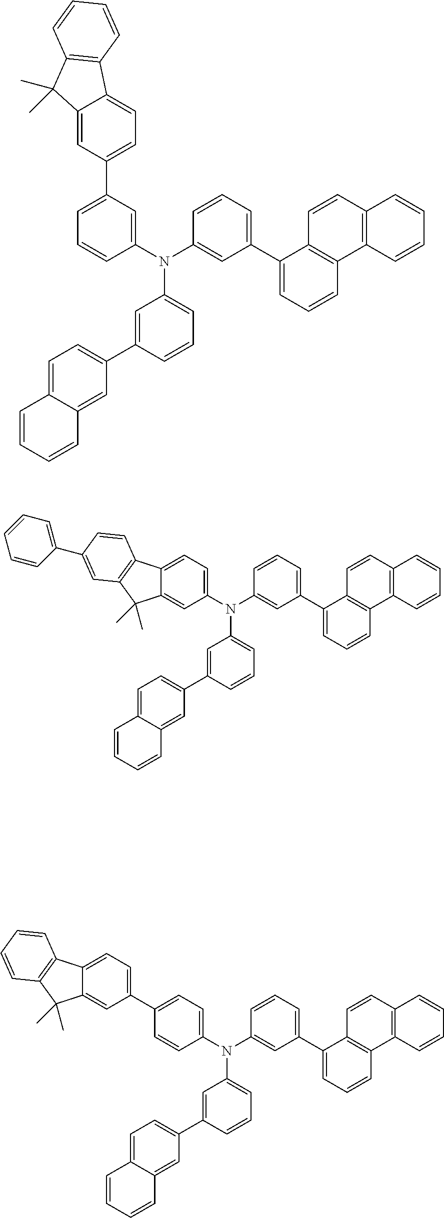 [SMILES: CC1(C)c2ccccc2-c2ccc(-c3cccc(N(c4cccc(-c5ccc6ccccc6c5)c4)c4cccc(-c5cccc6c5ccc5ccccc56)c4)c3)cc21 ; CC1(C)c2cc(-c3ccccc3)ccc2-c2ccc(N(c3cccc(-c4ccc5ccccc5c4)c3)c3cccc(-c4cccc5c4ccc4ccccc45)c3)cc21 ; CC1(C)c2ccccc2-c2ccc(-c3ccc(N(c4cccc(-c5ccc6ccccc6c5)c4)c4cccc(-c5cccc6c5ccc5ccccc56)c4)cc3)cc21]

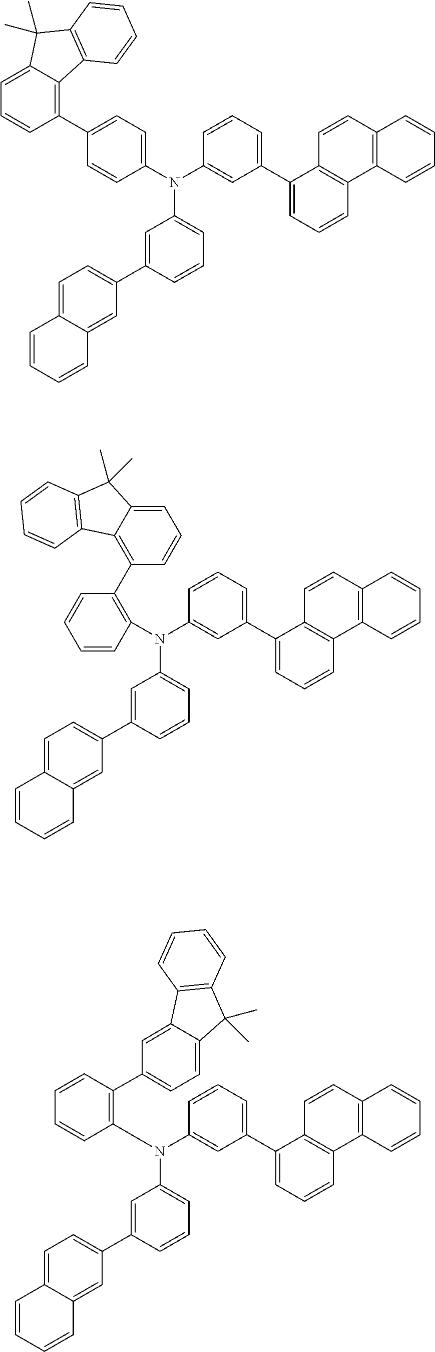 [SMILES: CC1(C)c2ccccc2-c2c(-c3ccc(N(c4cccc(-c5ccc6ccccc6c5)c4)c4cccc(-c5cccc6c5ccc5ccccc56)c4)cc3)cccc21 ; CC1(C)c2ccccc2-c2c(-c3ccccc3N(c3cccc(-c4ccc5ccccc5c4)c3)c3cccc(-c4cccc5c4ccc4ccccc45)c3)cccc21 ; CC1(C)c2ccccc2-c2cc(-c3ccccc3N(c3cccc(-c4ccc5ccccc5c4)c3)c3cccc(-c4cccc5c4ccc4ccccc45)c3)ccc21]

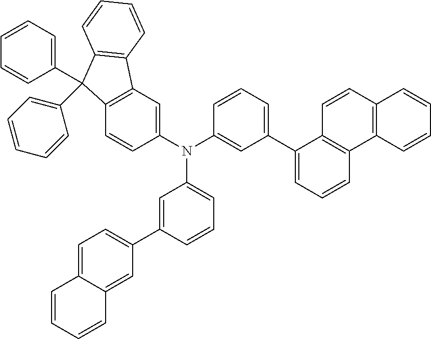 [SMILES: c1ccc(C2(c3ccccc3)c3ccccc3-c3cc(N(c4cccc(-c5ccc6ccccc6c5)c4)c4cccc(-c5cccc6c5ccc5ccccc56)c4)ccc32)cc1]

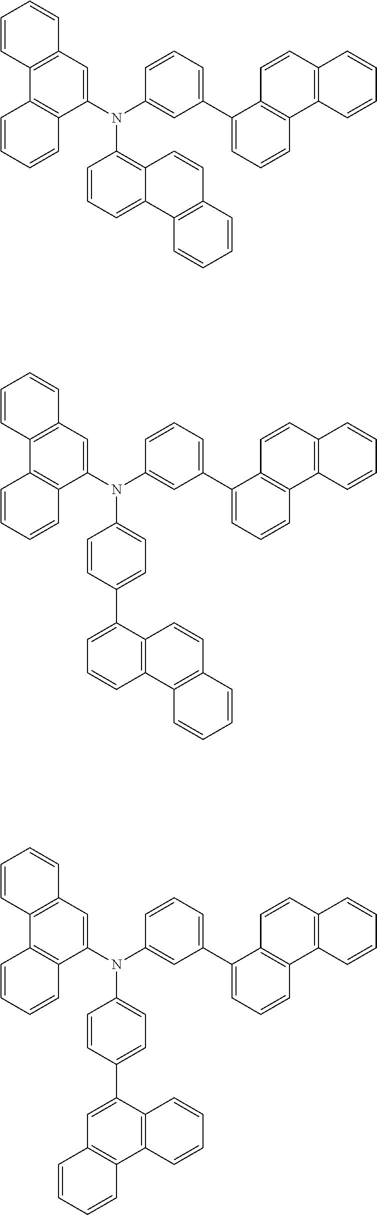 [SMILES: c1cc(-c2cccc3c2ccc2ccccc23)cc(N(c2cccc3c2ccc2ccccc23)c2cc3ccccc3c3ccccc23)c1 ; c1cc(-c2cccc3c2ccc2ccccc23)cc(N(c2ccc(-c3cccc4c3ccc3ccccc34)cc2)c2cc3ccccc3c3ccccc23)c1 ; c1cc(-c2cccc3c2ccc2ccccc23)cc(N(c2ccc(-c3cc4ccccc4c4ccccc34)cc2)c2cc3ccccc3c3ccccc23)c1]

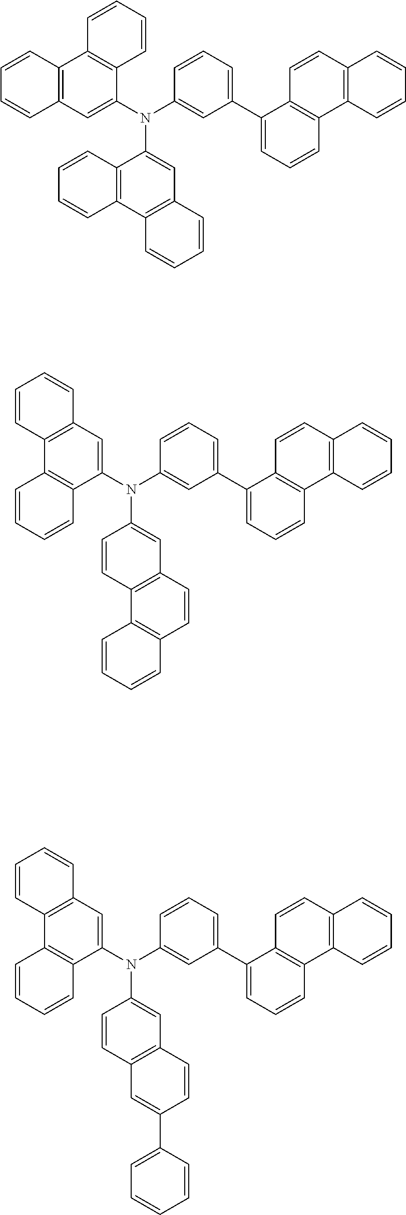 [SMILES: c1cc(-c2cccc3c2ccc2ccccc23)cc(N(c2cc3ccccc3c3ccccc23)c2cc3ccccc3c3ccccc23)c1 ; c1cc(-c2cccc3c2ccc2ccccc23)cc(N(c2ccc3c(ccc4ccccc43)c2)c2cc3ccccc3c3ccccc23)c1 ; c1ccc(-c2ccc3cc(N(c4cccc(-c5cccc6c5ccc5ccccc56)c4)c4cc5ccccc5c5ccccc45)ccc3c2)cc1]

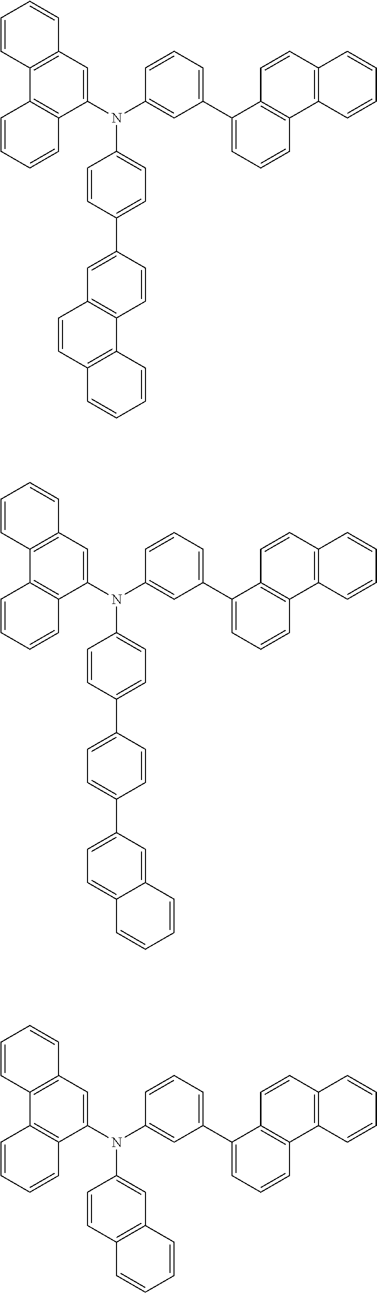 [SMILES: c1cc(-c2cccc3c2ccc2ccccc23)cc(N(c2ccc(-c3ccc4c(ccc5ccccc54)c3)cc2)c2cc3ccccc3c3ccccc23)c1 ; c1cc(-c2cccc3c2ccc2ccccc23)cc(N(c2ccc(-c3ccc(-c4ccc5ccccc5c4)cc3)cc2)c2cc3ccccc3c3ccccc23)c1 ; c1cc(-c2cccc3c2ccc2ccccc23)cc(N(c2ccc3ccccc3c2)c2cc3ccccc3c3ccccc23)c1]

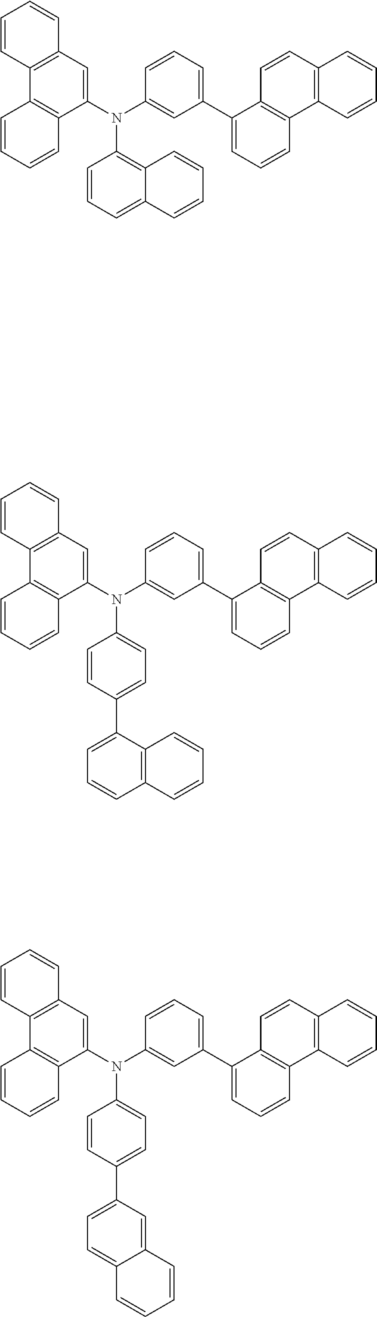 [SMILES: c1cc(-c2cccc3c2ccc2ccccc23)cc(N(c2cccc3ccccc23)c2cc3ccccc3c3ccccc23)c1 ; c1cc(-c2cccc3c2ccc2ccccc23)cc(N(c2ccc(-c3cccc4ccccc34)cc2)c2cc3ccccc3c3ccccc23)c1 ; c1cc(-c2cccc3c2ccc2ccccc23)cc(N(c2ccc(-c3ccc4ccccc4c3)cc2)c2cc3ccccc3c3ccccc23)c1]

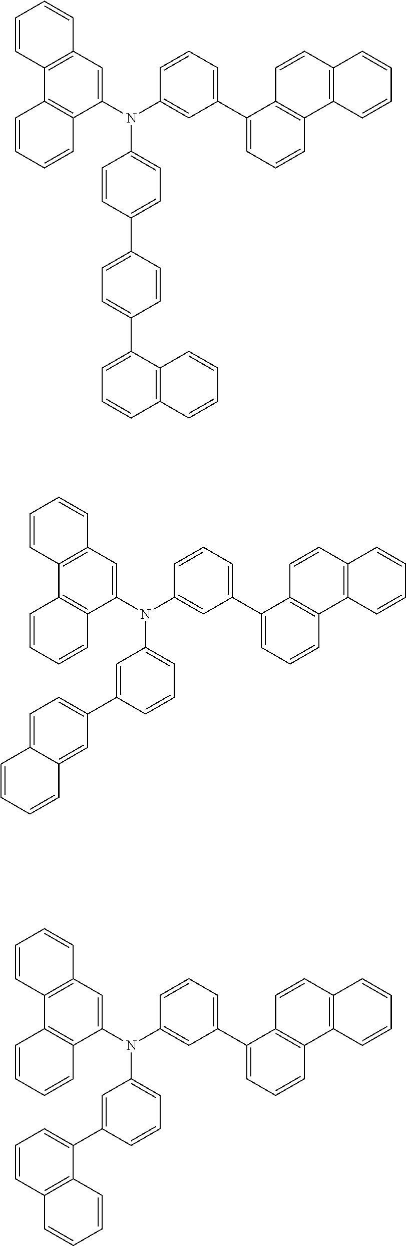 [SMILES: c1cc(-c2cccc3c2ccc2ccccc23)cc(N(c2ccc(-c3ccc(-c4cccc5ccccc45)cc3)cc2)c2cc3ccccc3c3ccccc23)c1 ; c1cc(-c2ccc3ccccc3c2)cc(N(c2cccc(-c3cccc4c3ccc3ccccc34)c2)c2cc3ccccc3c3ccccc23)c1 ; c1cc(-c2cccc3ccccc23)cc(N(c2cccc(-c3cccc4c3ccc3ccccc34)c2)c2cc3ccccc3c3ccccc23)c1]

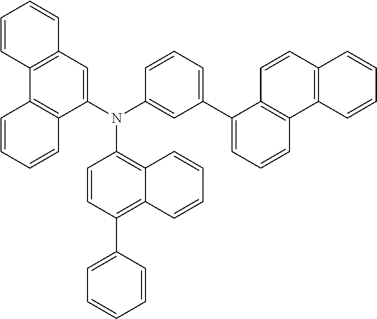 [SMILES: c1ccc(-c2ccc(N(c3cccc(-c4cccc5c4ccc4ccccc45)c3)c3cc4ccccc4c4ccccc34)c3ccccc23)cc1]

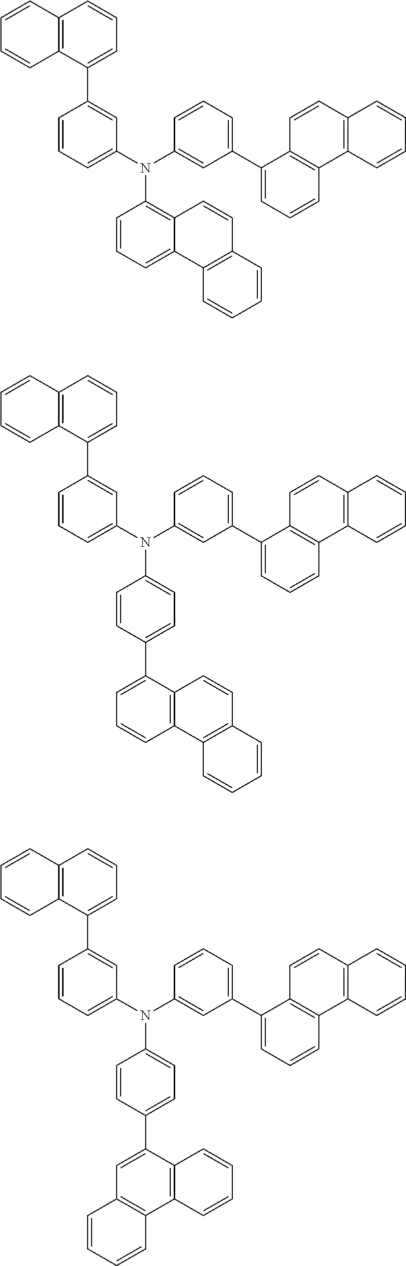 [SMILES: c1cc(-c2cccc3ccccc23)cc(N(c2cccc(-c3cccc4c3ccc3ccccc34)c2)c2cccc3c2ccc2ccccc23)c1 ; c1cc(-c2cccc3ccccc23)cc(N(c2ccc(-c3cccc4c3ccc3ccccc34)cc2)c2cccc(-c3cccc4c3ccc3ccccc34)c2)c1 ; c1cc(-c2cccc3ccccc23)cc(N(c2ccc(-c3cc4ccccc4c4ccccc34)cc2)c2cccc(-c3cccc4c3ccc3ccccc34)c2)c1]

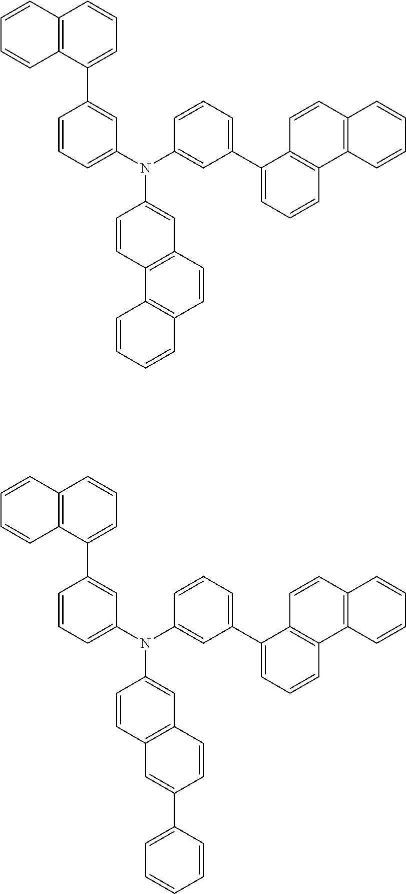 [SMILES: c1cc(-c2cccc3ccccc23)cc(N(c2cccc(-c3cccc4c3ccc3ccccc34)c2)c2ccc3c(ccc4ccccc43)c2)c1 ; c1ccc(-c2ccc3cc(N(c4cccc(-c5cccc6ccccc56)c4)c4cccc(-c5cccc6c5ccc5ccccc56)c4)ccc3c2)cc1]

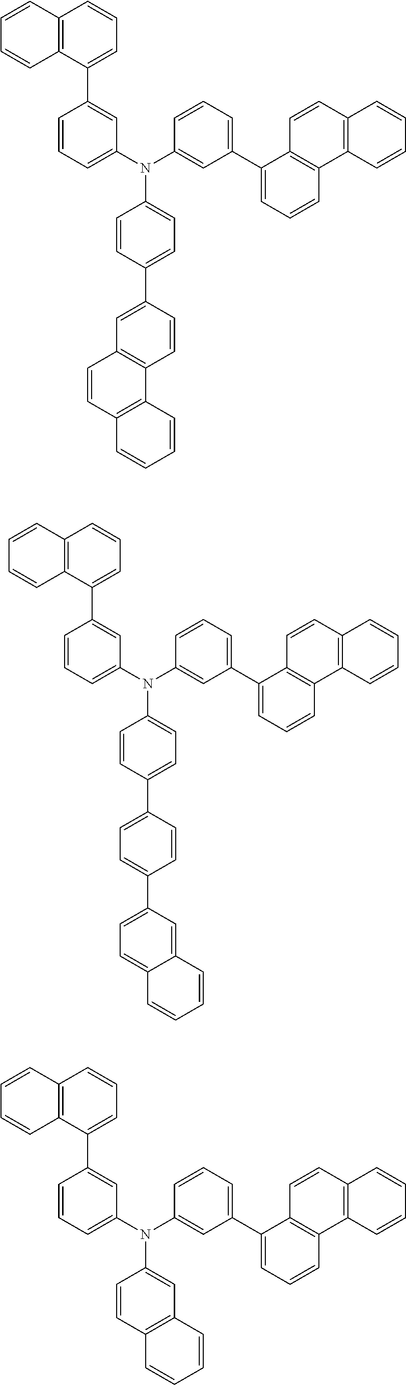 [SMILES: c1cc(-c2cccc3ccccc23)cc(N(c2ccc(-c3ccc4c(ccc5ccccc54)c3)cc2)c2cccc(-c3cccc4c3ccc3ccccc34)c2)c1 ; c1cc(-c2cccc3ccccc23)cc(N(c2ccc(-c3ccc(-c4ccc5ccccc5c4)cc3)cc2)c2cccc(-c3cccc4c3ccc3ccccc34)c2)c1 ; c1cc(-c2cccc3ccccc23)cc(N(c2cccc(-c3cccc4c3ccc3ccccc34)c2)c2ccc3ccccc3c2)c1]

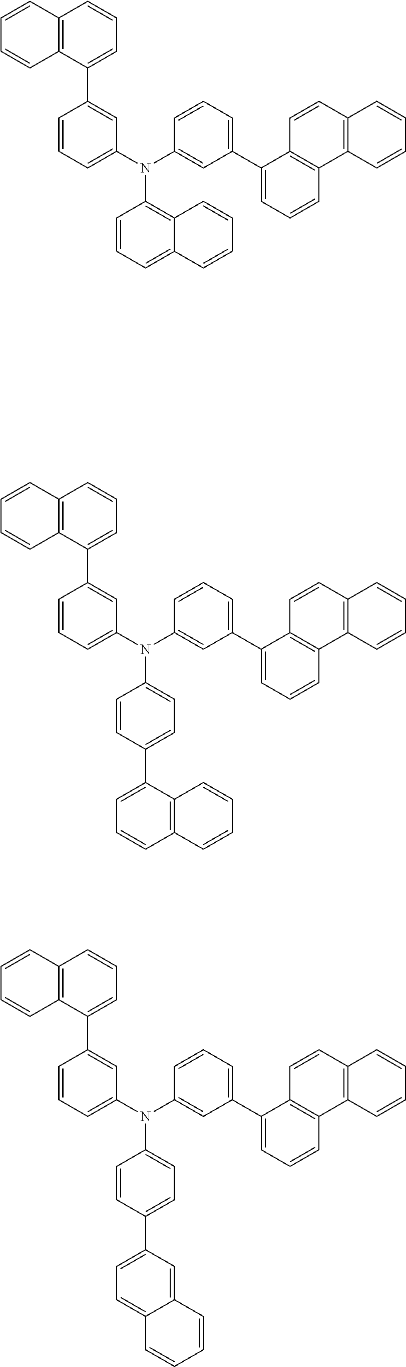 [SMILES: c1cc(-c2cccc3ccccc23)cc(N(c2cccc(-c3cccc4c3ccc3ccccc34)c2)c2cccc3ccccc23)c1 ; c1cc(-c2cccc3ccccc23)cc(N(c2ccc(-c3cccc4ccccc34)cc2)c2cccc(-c3cccc4c3ccc3ccccc34)c2)c1 ; c1cc(-c2cccc3ccccc23)cc(N(c2ccc(-c3ccc4ccccc4c3)cc2)c2cccc(-c3cccc4c3ccc3ccccc34)c2)c1]

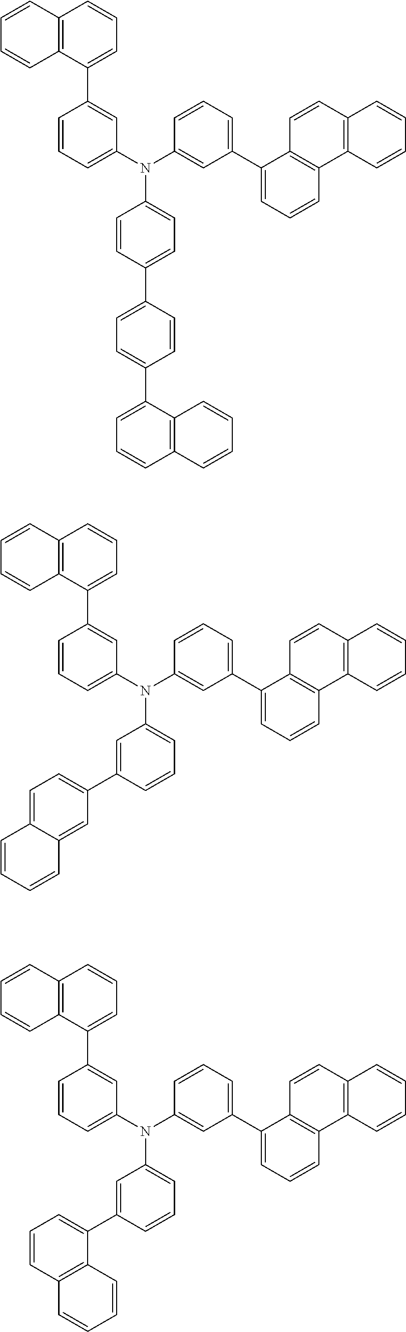 [SMILES: c1cc(-c2cccc3ccccc23)cc(N(c2ccc(-c3ccc(-c4cccc5ccccc45)cc3)cc2)c2cccc(-c3cccc4c3ccc3ccccc34)c2)c1 ; c1cc(-c2ccc3ccccc3c2)cc(N(c2cccc(-c3cccc4ccccc34)c2)c2cccc(-c3cccc4c3ccc3ccccc34)c2)c1 ; c1cc(-c2cccc3ccccc23)cc(N(c2cccc(-c3cccc4ccccc34)c2)c2cccc(-c3cccc4c3ccc3ccccc34)c2)c1]

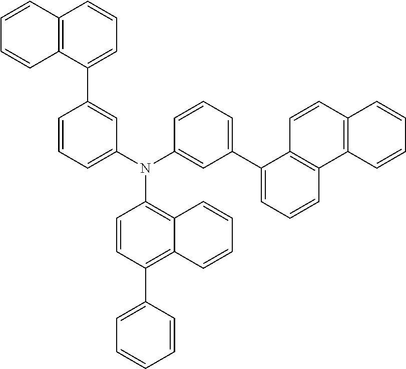 [SMILES: c1ccc(-c2ccc(N(c3cccc(-c4cccc5ccccc45)c3)c3cccc(-c4cccc5c4ccc4ccccc45)c3)c3ccccc23)cc1]

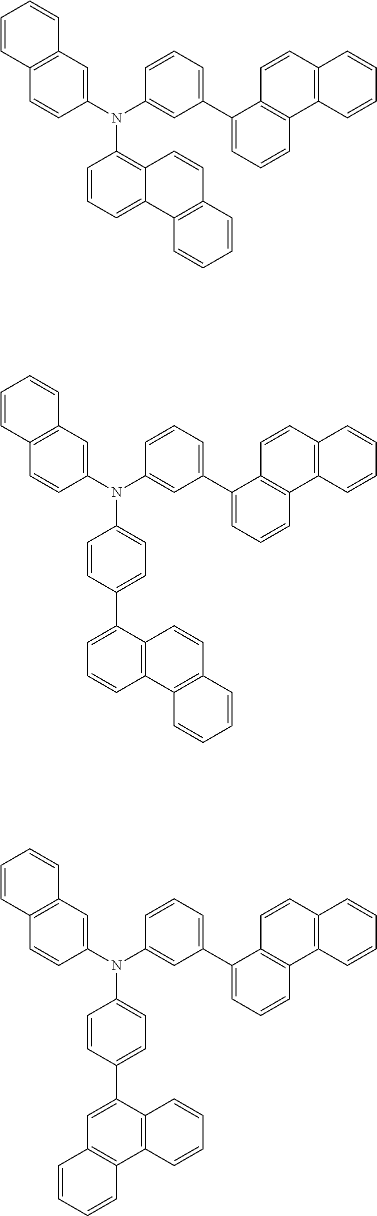 [SMILES: c1cc(-c2cccc3c2ccc2ccccc23)cc(N(c2ccc3ccccc3c2)c2cccc3c2ccc2ccccc23)c1 ; c1cc(-c2cccc3c2ccc2ccccc23)cc(N(c2ccc(-c3cccc4c3ccc3ccccc34)cc2)c2ccc3ccccc3c2)c1 ; c1cc(-c2cccc3c2ccc2ccccc23)cc(N(c2ccc(-c3cc4ccccc4c4ccccc34)cc2)c2ccc3ccccc3c2)c1]

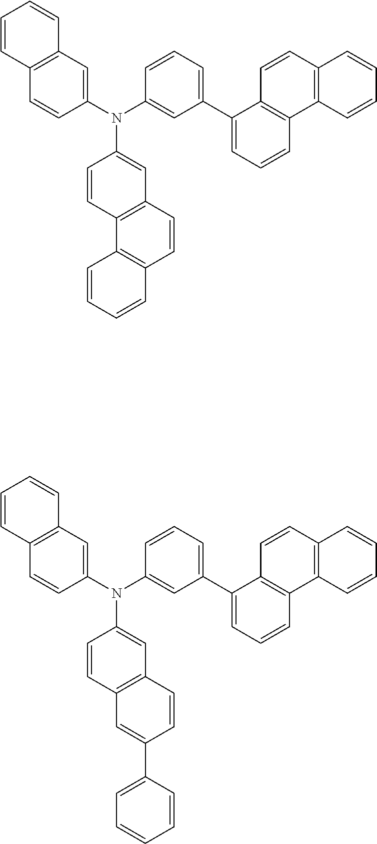 [SMILES: c1cc(-c2cccc3c2ccc2ccccc23)cc(N(c2ccc3ccccc3c2)c2ccc3c(ccc4ccccc43)c2)c1 ; c1ccc(-c2ccc3cc(N(c4cccc(-c5cccc6c5ccc5ccccc56)c4)c4ccc5ccccc5c4)ccc3c2)cc1]

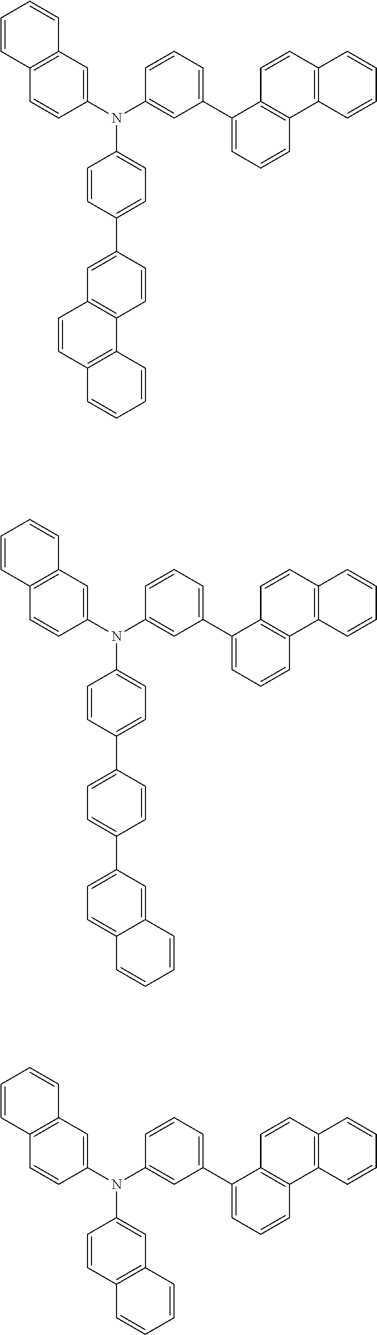 [SMILES: c1cc(-c2cccc3c2ccc2ccccc23)cc(N(c2ccc(-c3ccc4c(ccc5ccccc54)c3)cc2)c2ccc3ccccc3c2)c1 ; c1cc(-c2cccc3c2ccc2ccccc23)cc(N(c2ccc(-c3ccc(-c4ccc5ccccc5c4)cc3)cc2)c2ccc3ccccc3c2)c1 ; c1cc(-c2cccc3c2ccc2ccccc23)cc(N(c2ccc3ccccc3c2)c2ccc3ccccc3c2)c1]

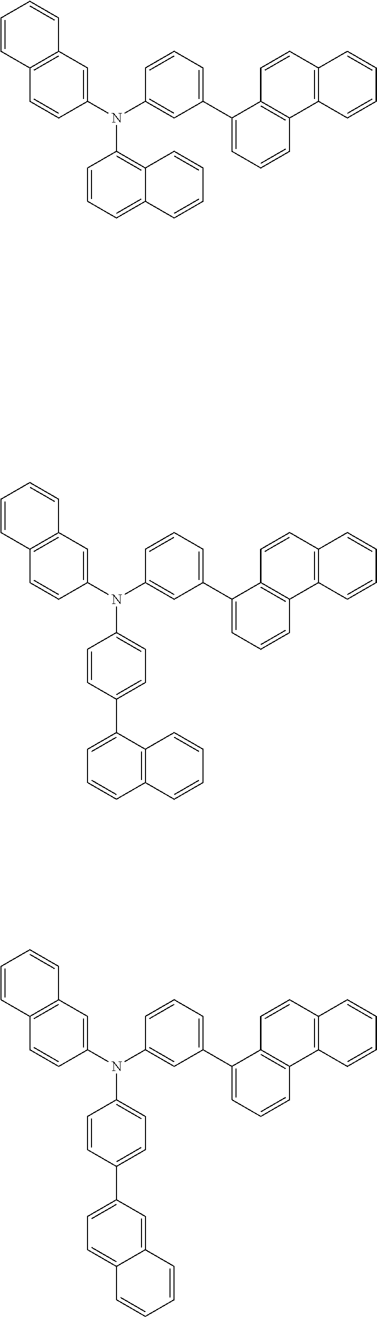 [SMILES: c1cc(-c2cccc3c2ccc2ccccc23)cc(N(c2ccc3ccccc3c2)c2cccc3ccccc23)c1 ; c1cc(-c2cccc3c2ccc2ccccc23)cc(N(c2ccc(-c3cccc4ccccc34)cc2)c2ccc3ccccc3c2)c1 ; c1cc(-c2cccc3c2ccc2ccccc23)cc(N(c2ccc(-c3ccc4ccccc4c3)cc2)c2ccc3ccccc3c2)c1]

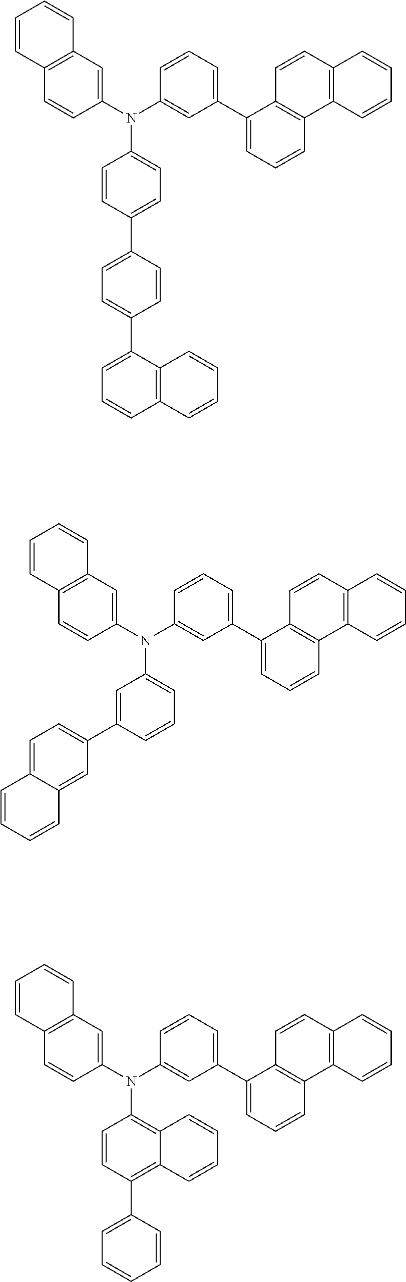 [SMILES: c1cc(-c2cccc3c2ccc2ccccc23)cc(N(c2ccc(-c3ccc(-c4cccc5ccccc45)cc3)cc2)c2ccc3ccccc3c2)c1 ; c1cc(-c2ccc3ccccc3c2)cc(N(c2cccc(-c3cccc4c3ccc3ccccc34)c2)c2ccc3ccccc3c2)c1 ; c1ccc(-c2ccc(N(c3cccc(-c4cccc5c4ccc4ccccc45)c3)c3ccc4ccccc4c3)c3ccccc23)cc1]

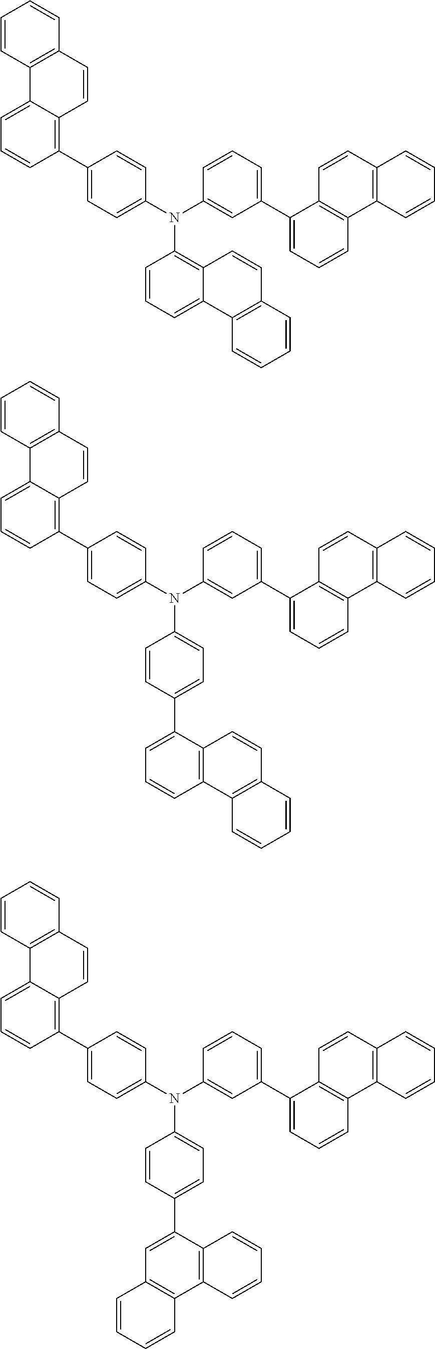 [SMILES: c1cc(-c2cccc3c2ccc2ccccc23)cc(N(c2ccc(-c3cccc4c3ccc3ccccc34)cc2)c2cccc3c2ccc2ccccc23)c1 ; c1cc(-c2cccc3c2ccc2ccccc23)cc(N(c2ccc(-c3cccc4c3ccc3ccccc34)cc2)c2ccc(-c3cccc4c3ccc3ccccc34)cc2)c1 ; c1cc(-c2cccc3c2ccc2ccccc23)cc(N(c2ccc(-c3cccc4c3ccc3ccccc34)cc2)c2ccc(-c3cc4ccccc4c4ccccc34)cc2)c1]

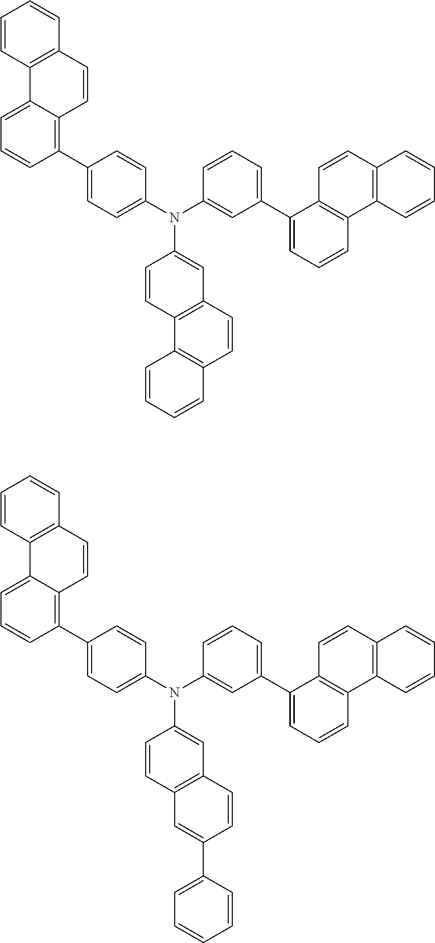 [SMILES: c1cc(-c2cccc3c2ccc2ccccc23)cc(N(c2ccc(-c3cccc4c3ccc3ccccc34)cc2)c2ccc3c(ccc4ccccc43)c2)c1 ; c1ccc(-c2ccc3cc(N(c4ccc(-c5cccc6c5ccc5ccccc56)cc4)c4cccc(-c5cccc6c5ccc5ccccc56)c4)ccc3c2)cc1]

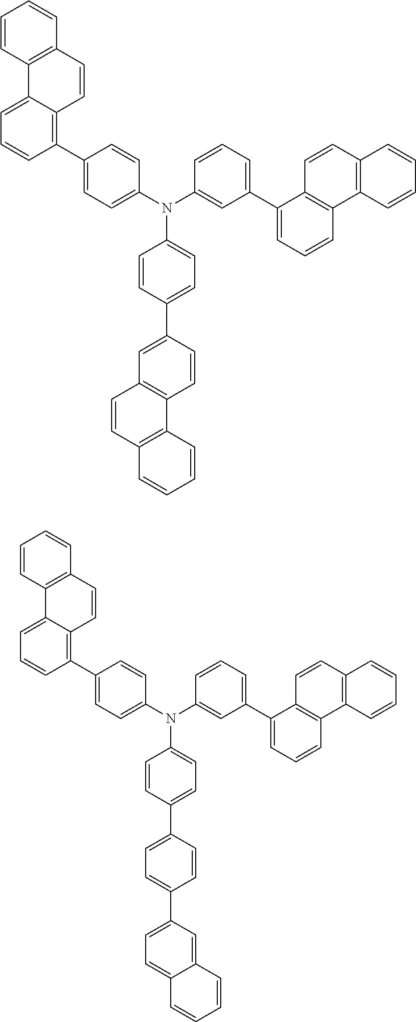 [SMILES: c1cc(-c2cccc3c2ccc2ccccc23)cc(N(c2ccc(-c3ccc4c(ccc5ccccc54)c3)cc2)c2ccc(-c3cccc4c3ccc3ccccc34)cc2)c1 ; c1cc(-c2cccc3c2ccc2ccccc23)cc(N(c2ccc(-c3ccc(-c4ccc5ccccc5c4)cc3)cc2)c2ccc(-c3cccc4c3ccc3ccccc34)cc2)c1]

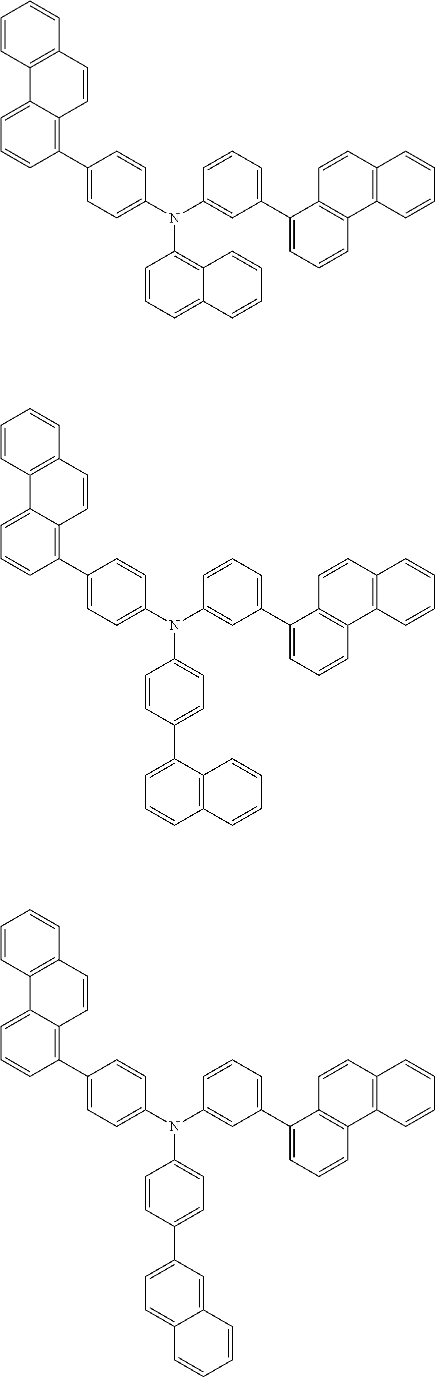 [SMILES: c1cc(-c2cccc3c2ccc2ccccc23)cc(N(c2ccc(-c3cccc4c3ccc3ccccc34)cc2)c2cccc3ccccc23)c1 ; c1cc(-c2cccc3c2ccc2ccccc23)cc(N(c2ccc(-c3cccc4ccccc34)cc2)c2ccc(-c3cccc4c3ccc3ccccc34)cc2)c1 ; c1cc(-c2cccc3c2ccc2ccccc23)cc(N(c2ccc(-c3ccc4ccccc4c3)cc2)c2ccc(-c3cccc4c3ccc3ccccc34)cc2)c1]

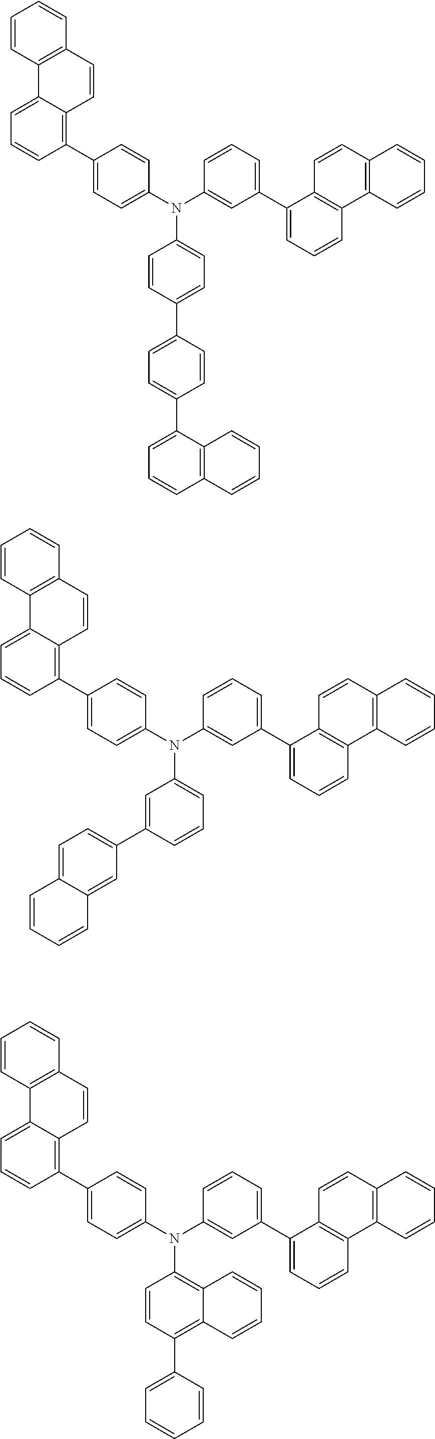 [SMILES: c1cc(-c2cccc3c2ccc2ccccc23)cc(N(c2ccc(-c3ccc(-c4cccc5ccccc45)cc3)cc2)c2ccc(-c3cccc4c3ccc3ccccc34)cc2)c1 ; c1cc(-c2ccc3ccccc3c2)cc(N(c2ccc(-c3cccc4c3ccc3ccccc34)cc2)c2cccc(-c3cccc4c3ccc3ccccc34)c2)c1 ; c1ccc(-c2ccc(N(c3ccc(-c4cccc5c4ccc4ccccc45)cc3)c3cccc(-c4cccc5c4ccc4ccccc45)c3)c3ccccc23)cc1]

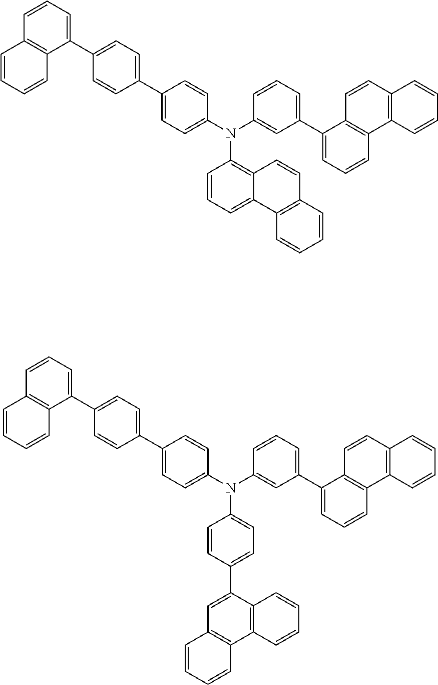 [SMILES: c1cc(-c2cccc3c2ccc2ccccc23)cc(N(c2ccc(-c3ccc(-c4cccc5ccccc45)cc3)cc2)c2cccc3c2ccc2ccccc23)c1 ; c1cc(-c2cccc3c2ccc2ccccc23)cc(N(c2ccc(-c3ccc(-c4cccc5ccccc45)cc3)cc2)c2ccc(-c3cc4ccccc4c4ccccc34)cc2)c1]

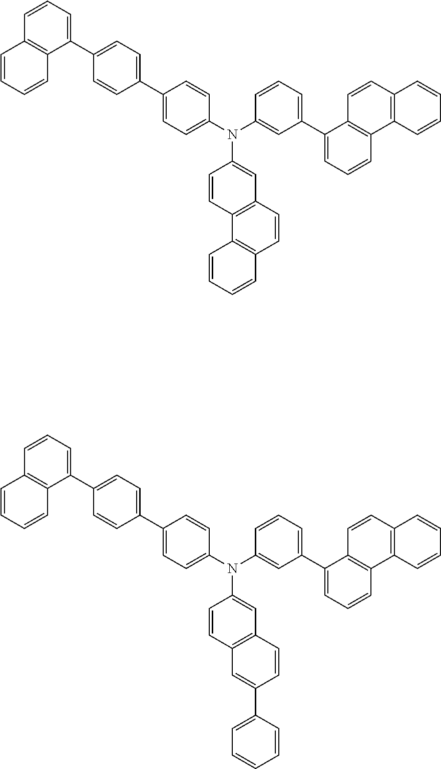 [SMILES: c1cc(-c2cccc3c2ccc2ccccc23)cc(N(c2ccc(-c3ccc(-c4cccc5ccccc45)cc3)cc2)c2ccc3c(ccc4ccccc43)c2)c1 ; c1ccc(-c2ccc3cc(N(c4ccc(-c5ccc(-c6cccc7ccccc67)cc5)cc4)c4cccc(-c5cccc6c5ccc5ccccc56)c4)ccc3c2)cc1]

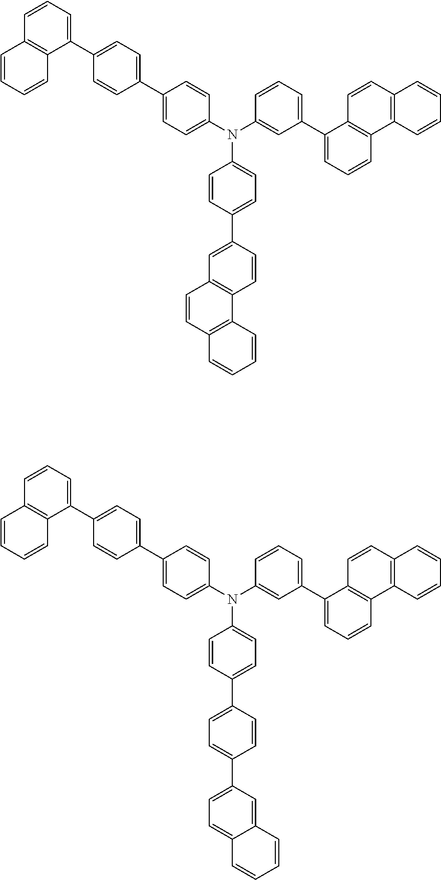 [SMILES: c1cc(-c2cccc3c2ccc2ccccc23)cc(N(c2ccc(-c3ccc(-c4cccc5ccccc45)cc3)cc2)c2ccc(-c3ccc4c(ccc5ccccc54)c3)cc2)c1 ; c1cc(-c2cccc3c2ccc2ccccc23)cc(N(c2ccc(-c3ccc(-c4ccc5ccccc5c4)cc3)cc2)c2ccc(-c3ccc(-c4cccc5ccccc45)cc3)cc2)c1]

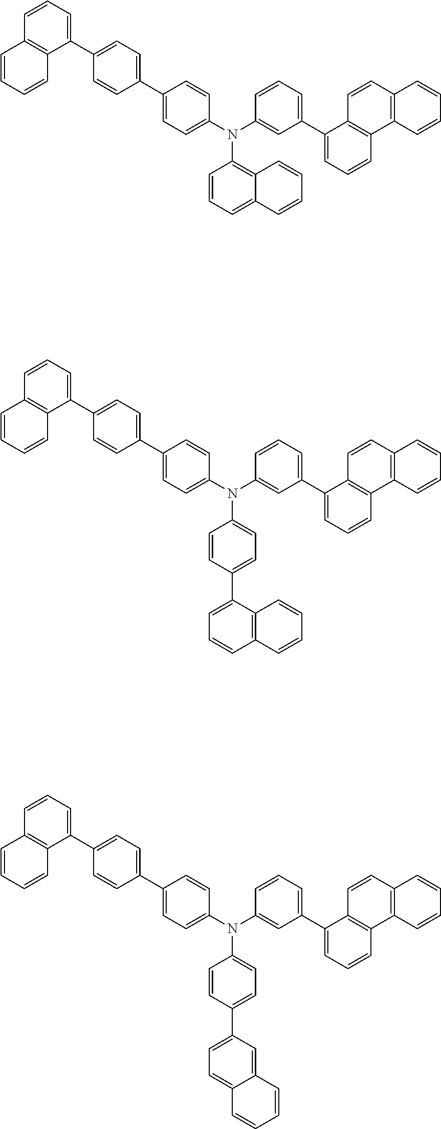 [SMILES: c1cc(-c2cccc3c2ccc2ccccc23)cc(N(c2ccc(-c3ccc(-c4cccc5ccccc45)cc3)cc2)c2cccc3ccccc23)c1 ; c1cc(-c2cccc3c2ccc2ccccc23)cc(N(c2ccc(-c3ccc(-c4cccc5ccccc45)cc3)cc2)c2ccc(-c3cccc4ccccc34)cc2)c1 ; c1cc(-c2cccc3c2ccc2ccccc23)cc(N(c2ccc(-c3ccc(-c4cccc5ccccc45)cc3)cc2)c2ccc(-c3ccc4ccccc4c3)cc2)c1]

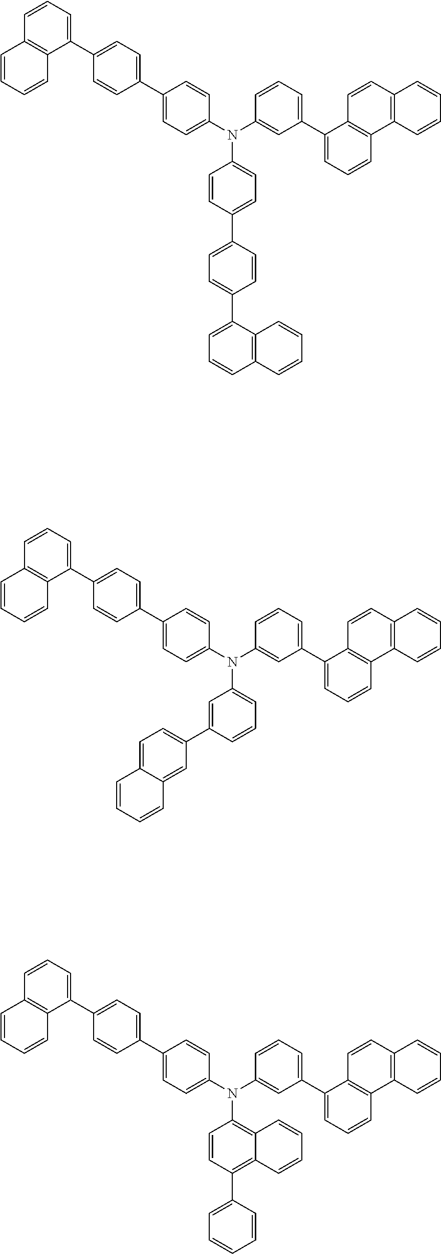 [SMILES: c1cc(-c2cccc3c2ccc2ccccc23)cc(N(c2ccc(-c3ccc(-c4cccc5ccccc45)cc3)cc2)c2ccc(-c3ccc(-c4cccc5ccccc45)cc3)cc2)c1 ; c1cc(-c2ccc3ccccc3c2)cc(N(c2ccc(-c3ccc(-c4cccc5ccccc45)cc3)cc2)c2cccc(-c3cccc4c3ccc3ccccc34)c2)c1 ; c1ccc(-c2ccc(N(c3ccc(-c4ccc(-c5cccc6ccccc56)cc4)cc3)c3cccc(-c4cccc5c4ccc4ccccc45)c3)c3ccccc23)cc1]

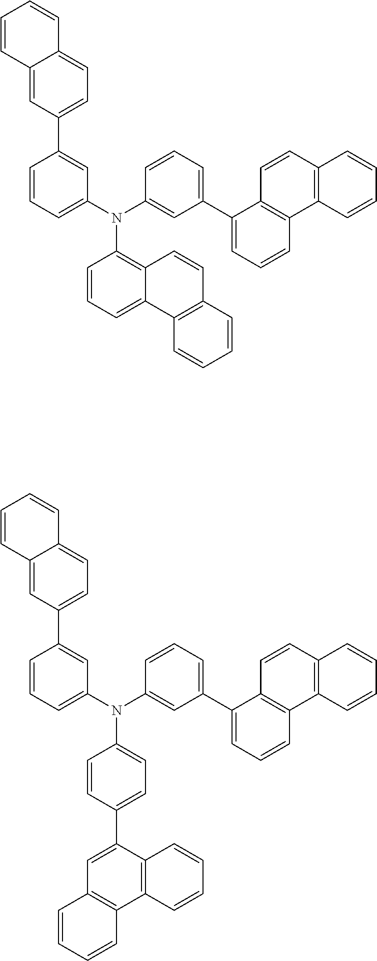 [SMILES: c1cc(-c2ccc3ccccc3c2)cc(N(c2cccc(-c3cccc4c3ccc3ccccc34)c2)c2cccc3c2ccc2ccccc23)c1 ; c1cc(-c2ccc3ccccc3c2)cc(N(c2ccc(-c3cc4ccccc4c4ccccc34)cc2)c2cccc(-c3cccc4c3ccc3ccccc34)c2)c1]

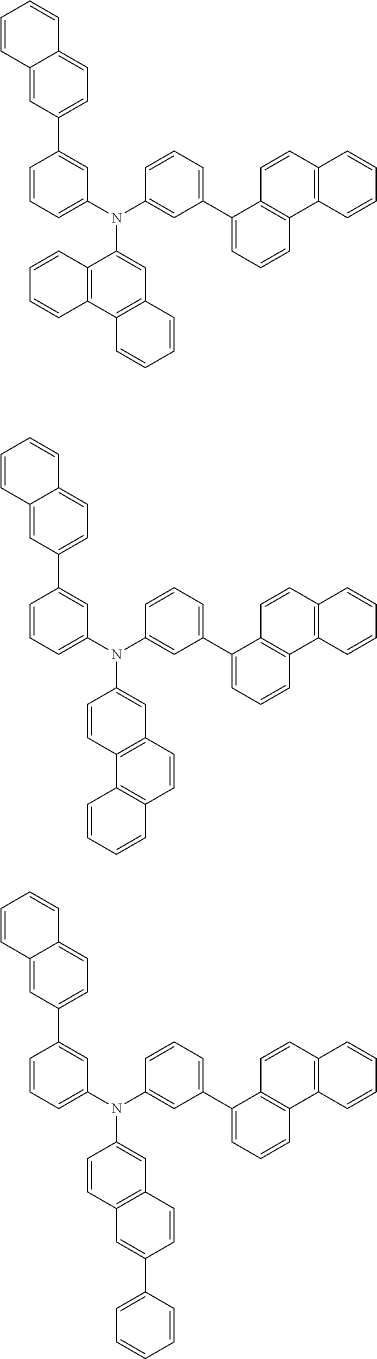 [SMILES: c1cc(-c2ccc3ccccc3c2)cc(N(c2cccc(-c3cccc4c3ccc3ccccc34)c2)c2cc3ccccc3c3ccccc23)c1 ; c1cc(-c2ccc3ccccc3c2)cc(N(c2cccc(-c3cccc4c3ccc3ccccc34)c2)c2ccc3c(ccc4ccccc43)c2)c1 ; c1ccc(-c2ccc3cc(N(c4cccc(-c5ccc6ccccc6c5)c4)c4cccc(-c5cccc6c5ccc5ccccc56)c4)ccc3c2)cc1]

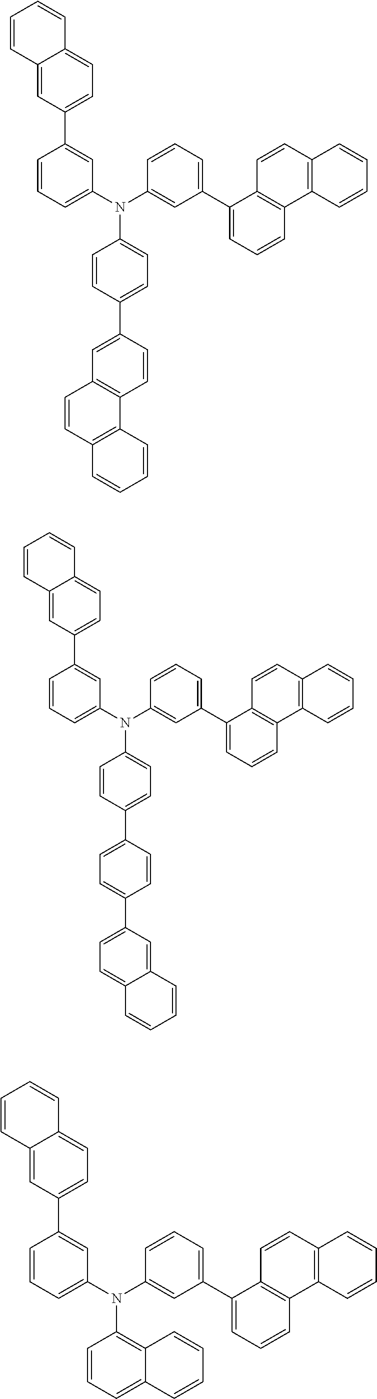 [SMILES: c1cc(-c2ccc3ccccc3c2)cc(N(c2ccc(-c3ccc4c(ccc5ccccc54)c3)cc2)c2cccc(-c3cccc4c3ccc3ccccc34)c2)c1 ; c1cc(-c2ccc3ccccc3c2)cc(N(c2ccc(-c3ccc(-c4ccc5ccccc5c4)cc3)cc2)c2cccc(-c3cccc4c3ccc3ccccc34)c2)c1 ; c1cc(-c2ccc3ccccc3c2)cc(N(c2cccc(-c3cccc4c3ccc3ccccc34)c2)c2cccc3ccccc23)c1]

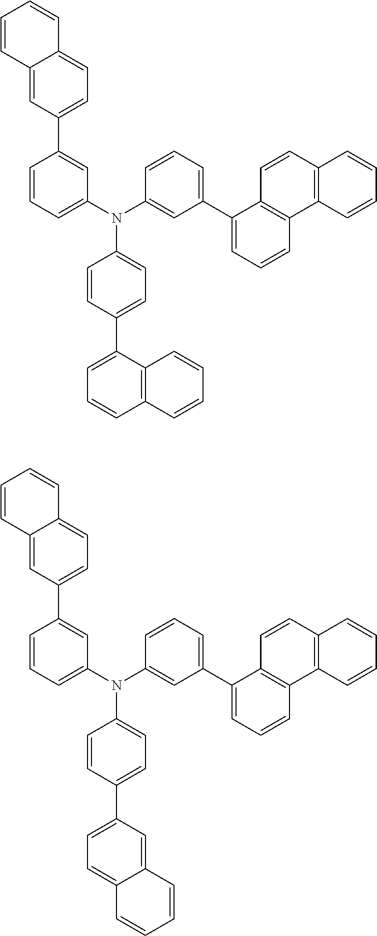 [SMILES: c1cc(-c2ccc3ccccc3c2)cc(N(c2ccc(-c3cccc4ccccc34)cc2)c2cccc(-c3cccc4c3ccc3ccccc34)c2)c1 ; c1cc(-c2ccc3ccccc3c2)cc(N(c2ccc(-c3ccc4ccccc4c3)cc2)c2cccc(-c3cccc4c3ccc3ccccc34)c2)c1]

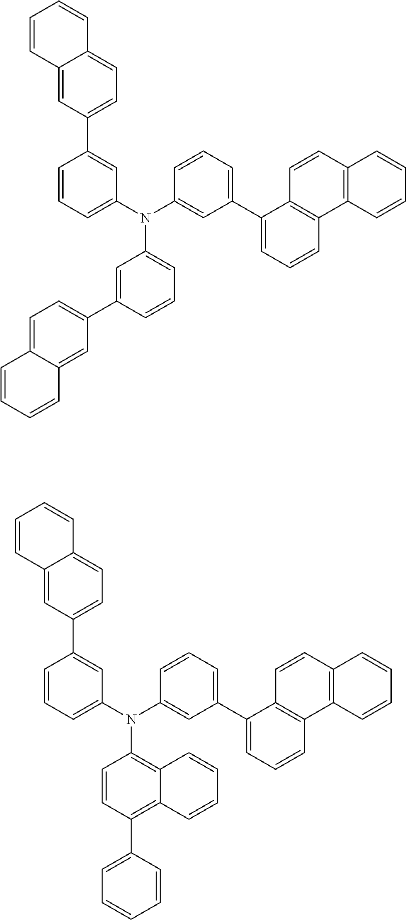 [SMILES: c1cc(-c2ccc3ccccc3c2)cc(N(c2cccc(-c3ccc4ccccc4c3)c2)c2cccc(-c3cccc4c3ccc3ccccc34)c2)c1 ; c1ccc(-c2ccc(N(c3cccc(-c4ccc5ccccc5c4)c3)c3cccc(-c4cccc5c4ccc4ccccc45)c3)c3ccccc23)cc1]

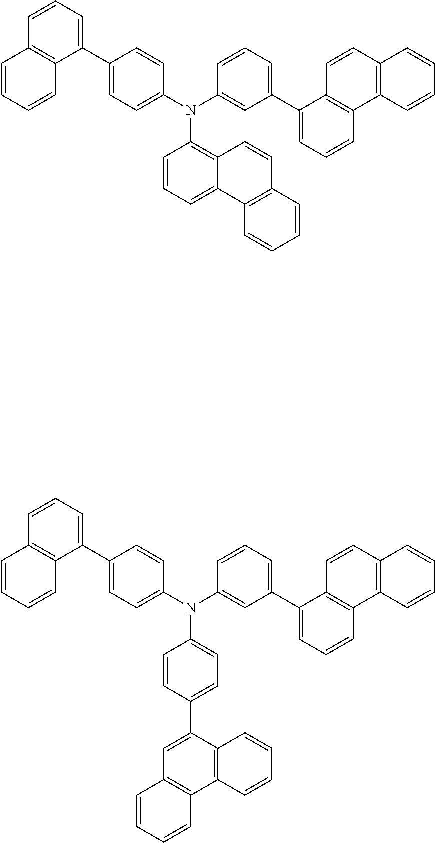 [SMILES: c1cc(-c2cccc3c2ccc2ccccc23)cc(N(c2ccc(-c3cccc4ccccc34)cc2)c2cccc3c2ccc2ccccc23)c1 ; c1cc(-c2cccc3c2ccc2ccccc23)cc(N(c2ccc(-c3cccc4ccccc34)cc2)c2ccc(-c3cc4ccccc4c4ccccc34)cc2)c1]

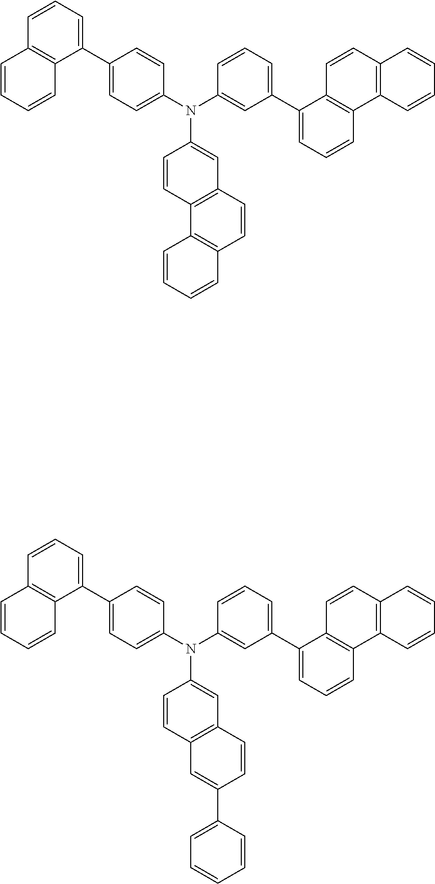 [SMILES: c1cc(-c2cccc3c2ccc2ccccc23)cc(N(c2ccc(-c3cccc4ccccc34)cc2)c2ccc3c(ccc4ccccc43)c2)c1 ; c1ccc(-c2ccc3cc(N(c4ccc(-c5cccc6ccccc56)cc4)c4cccc(-c5cccc6c5ccc5ccccc56)c4)ccc3c2)cc1]

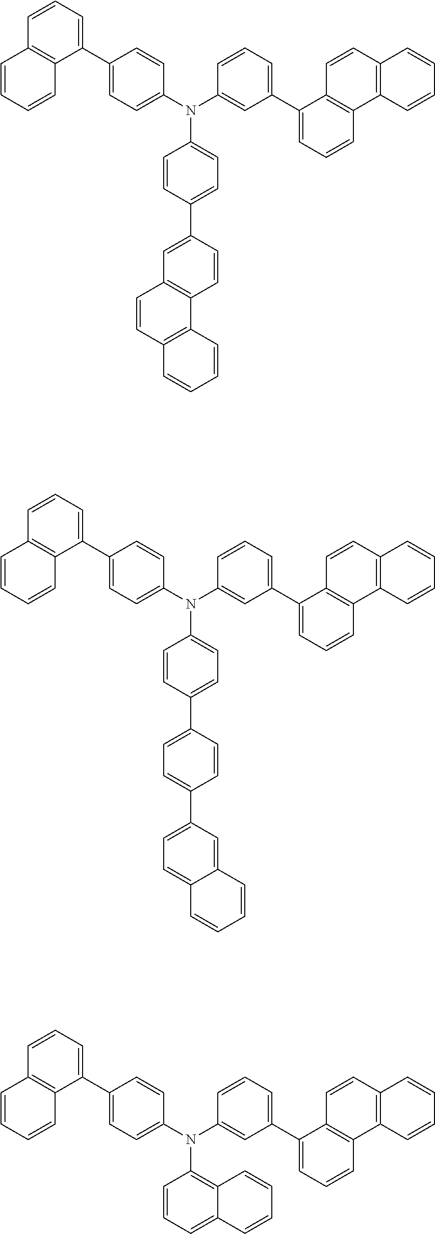 [SMILES: c1cc(-c2cccc3c2ccc2ccccc23)cc(N(c2ccc(-c3ccc4c(ccc5ccccc54)c3)cc2)c2ccc(-c3cccc4ccccc34)cc2)c1 ; c1cc(-c2cccc3c2ccc2ccccc23)cc(N(c2ccc(-c3ccc(-c4ccc5ccccc5c4)cc3)cc2)c2ccc(-c3cccc4ccccc34)cc2)c1 ; c1cc(-c2cccc3c2ccc2ccccc23)cc(N(c2ccc(-c3cccc4ccccc34)cc2)c2cccc3ccccc23)c1]

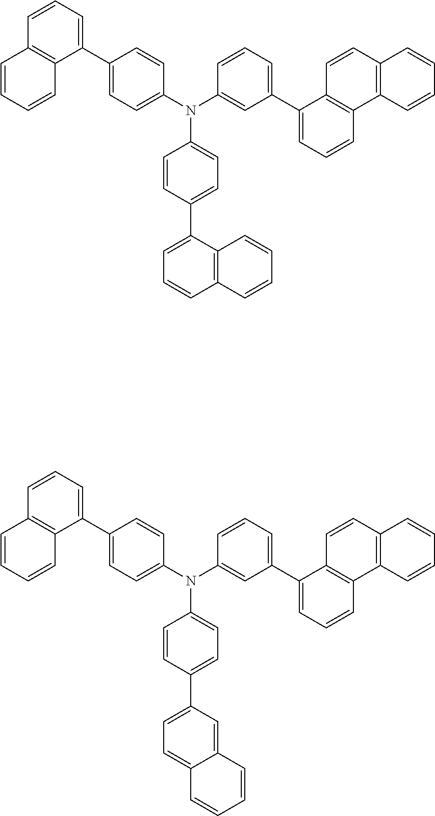 [SMILES: c1cc(-c2cccc3c2ccc2ccccc23)cc(N(c2ccc(-c3cccc4ccccc34)cc2)c2ccc(-c3cccc4ccccc34)cc2)c1 ; c1cc(-c2cccc3c2ccc2ccccc23)cc(N(c2ccc(-c3ccc4ccccc4c3)cc2)c2ccc(-c3cccc4ccccc34)cc2)c1]

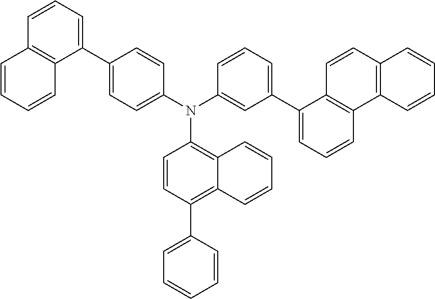 [SMILES: c1ccc(-c2ccc(N(c3ccc(-c4cccc5ccccc45)cc3)c3cccc(-c4cccc5c4ccc4ccccc45)c3)c3ccccc23)cc1]

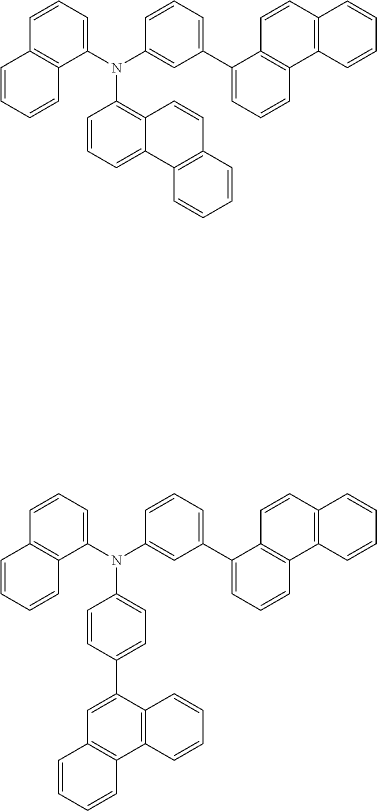 [SMILES: c1cc(-c2cccc3c2ccc2ccccc23)cc(N(c2cccc3ccccc23)c2cccc3c2ccc2ccccc23)c1 ; c1cc(-c2cccc3c2ccc2ccccc23)cc(N(c2ccc(-c3cc4ccccc4c4ccccc34)cc2)c2cccc3ccccc23)c1]

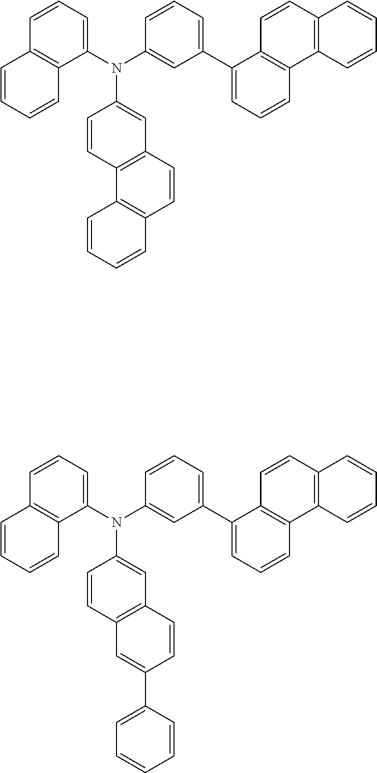 [SMILES: c1cc(-c2cccc3c2ccc2ccccc23)cc(N(c2ccc3c(ccc4ccccc43)c2)c2cccc3ccccc23)c1 ; c1ccc(-c2ccc3cc(N(c4cccc(-c5cccc6c5ccc5ccccc56)c4)c4cccc5ccccc45)ccc3c2)cc1]

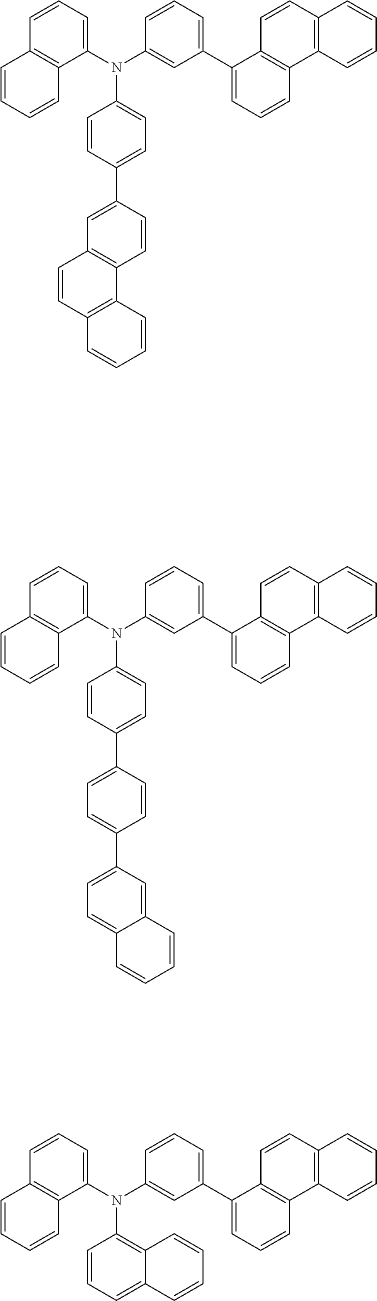 [SMILES: c1cc(-c2cccc3c2ccc2ccccc23)cc(N(c2ccc(-c3ccc4c(ccc5ccccc54)c3)cc2)c2cccc3ccccc23)c1 ; c1cc(-c2cccc3c2ccc2ccccc23)cc(N(c2ccc(-c3ccc(-c4ccc5ccccc5c4)cc3)cc2)c2cccc3ccccc23)c1 ; c1cc(-c2cccc3c2ccc2ccccc23)cc(N(c2cccc3ccccc23)c2cccc3ccccc23)c1]

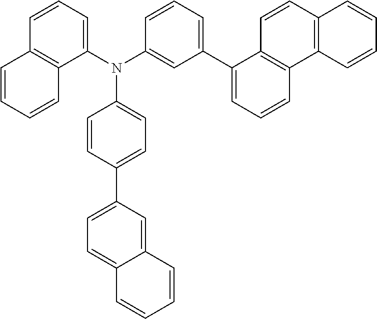 [SMILES: c1cc(-c2cccc3c2ccc2ccccc23)cc(N(c2ccc(-c3ccc4ccccc4c3)cc2)c2cccc3ccccc23)c1]

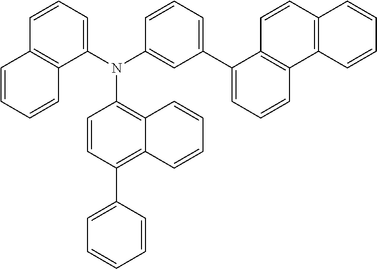 [SMILES: c1ccc(-c2ccc(N(c3cccc(-c4cccc5c4ccc4ccccc45)c3)c3cccc4ccccc34)c3ccccc23)cc1]

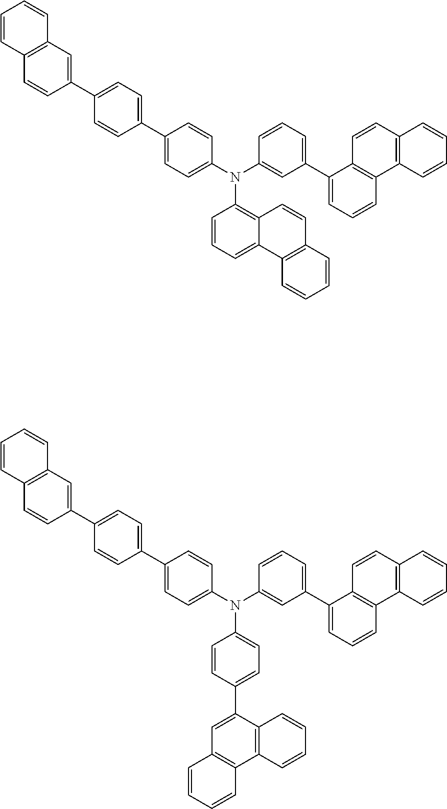 [SMILES: c1cc(-c2cccc3c2ccc2ccccc23)cc(N(c2ccc(-c3ccc(-c4ccc5ccccc5c4)cc3)cc2)c2cccc3c2ccc2ccccc23)c1 ; c1cc(-c2cccc3c2ccc2ccccc23)cc(N(c2ccc(-c3ccc(-c4ccc5ccccc5c4)cc3)cc2)c2ccc(-c3cc4ccccc4c4ccccc34)cc2)c1]

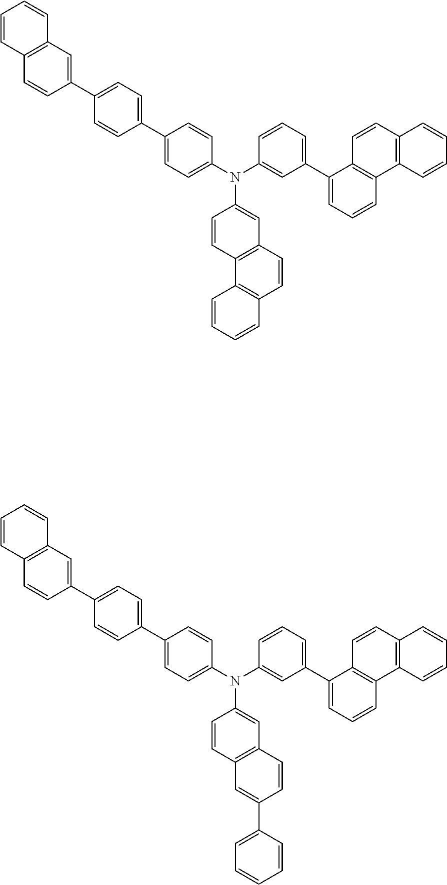 [SMILES: c1cc(-c2cccc3c2ccc2ccccc23)cc(N(c2ccc(-c3ccc(-c4ccc5ccccc5c4)cc3)cc2)c2ccc3c(ccc4ccccc43)c2)c1 ; c1ccc(-c2ccc3cc(N(c4ccc(-c5ccc(-c6ccc7ccccc7c6)cc5)cc4)c4cccc(-c5cccc6c5ccc5ccccc56)c4)ccc3c2)cc1]

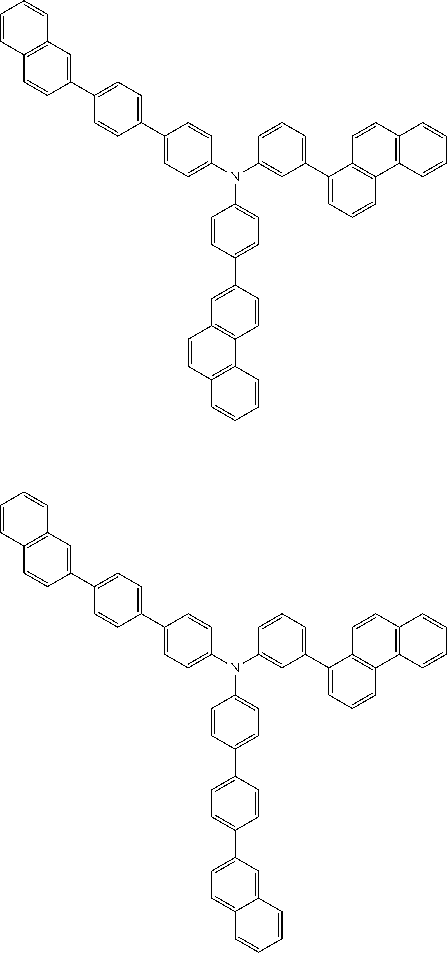 [SMILES: c1cc(-c2cccc3c2ccc2ccccc23)cc(N(c2ccc(-c3ccc(-c4ccc5ccccc5c4)cc3)cc2)c2ccc(-c3ccc4c(ccc5ccccc54)c3)cc2)c1 ; c1cc(-c2cccc3c2ccc2ccccc23)cc(N(c2ccc(-c3ccc(-c4ccc5ccccc5c4)cc3)cc2)c2ccc(-c3ccc(-c4ccc5ccccc5c4)cc3)cc2)c1]

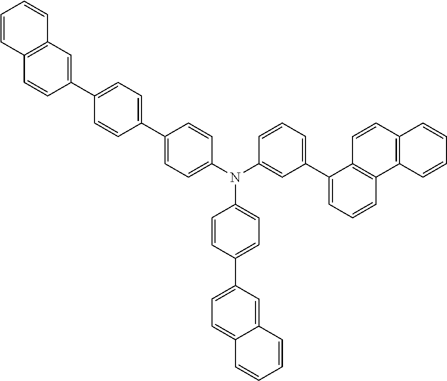 [SMILES: c1cc(-c2cccc3c2ccc2ccccc23)cc(N(c2ccc(-c3ccc(-c4ccc5ccccc5c4)cc3)cc2)c2ccc(-c3ccc4ccccc4c3)cc2)c1]

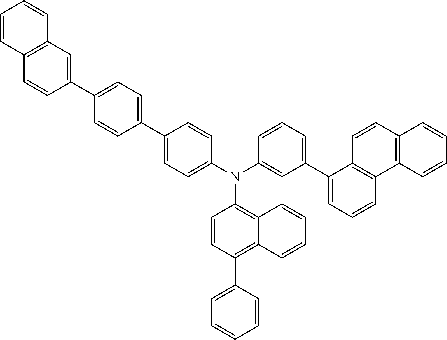 [SMILES: c1ccc(-c2ccc(N(c3ccc(-c4ccc(-c5ccc6ccccc6c5)cc4)cc3)c3cccc(-c4cccc5c4ccc4ccccc45)c3)c3ccccc23)cc1]

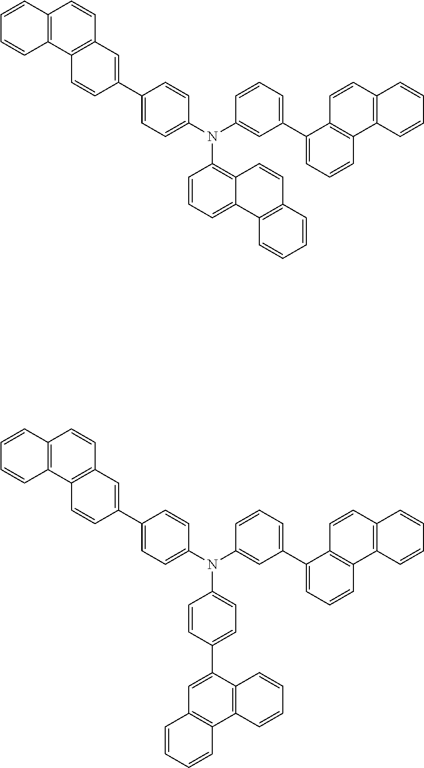 [SMILES: c1cc(-c2cccc3c2ccc2ccccc23)cc(N(c2ccc(-c3ccc4c(ccc5ccccc54)c3)cc2)c2cccc3c2ccc2ccccc23)c1 ; c1cc(-c2cccc3c2ccc2ccccc23)cc(N(c2ccc(-c3ccc4c(ccc5ccccc54)c3)cc2)c2ccc(-c3cc4ccccc4c4ccccc34)cc2)c1]

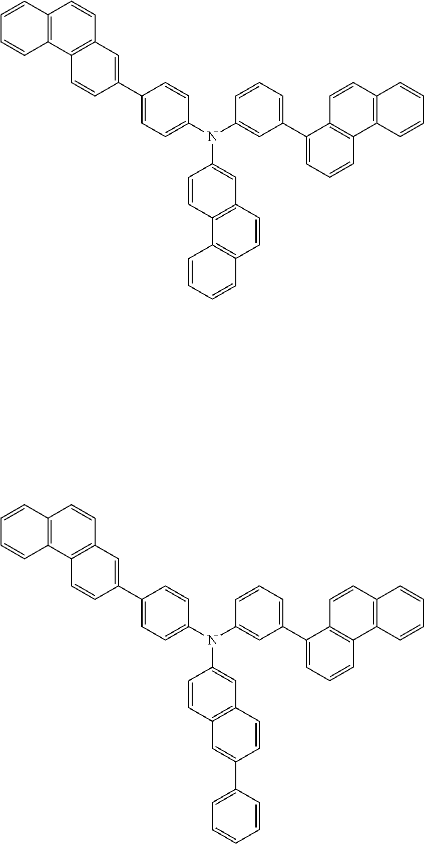 [SMILES: c1cc(-c2cccc3c2ccc2ccccc23)cc(N(c2ccc(-c3ccc4c(ccc5ccccc54)c3)cc2)c2ccc3c(ccc4ccccc43)c2)c1 ; c1ccc(-c2ccc3cc(N(c4ccc(-c5ccc6c(ccc7ccccc76)c5)cc4)c4cccc(-c5cccc6c5ccc5ccccc56)c4)ccc3c2)cc1]

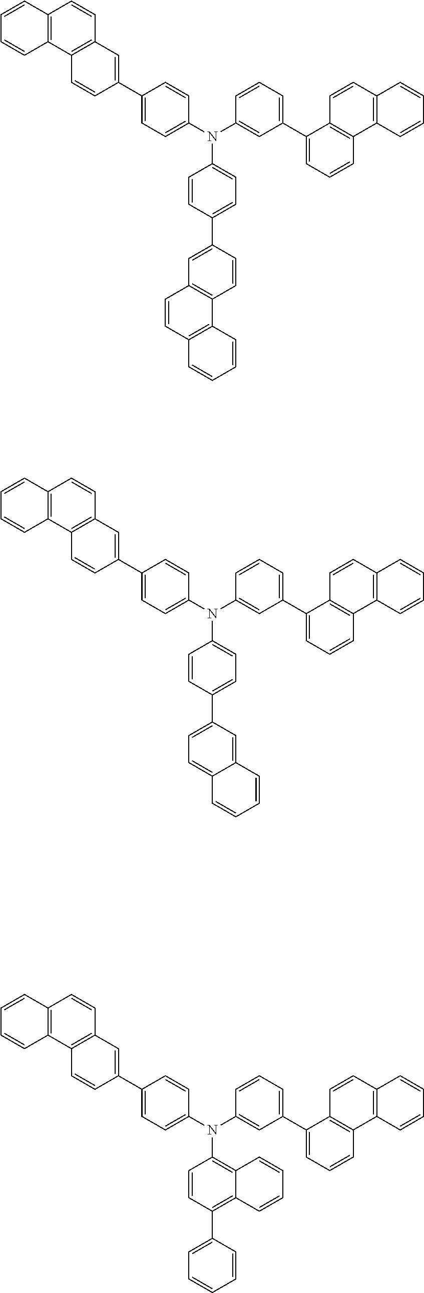 [SMILES: c1cc(-c2cccc3c2ccc2ccccc23)cc(N(c2ccc(-c3ccc4c(ccc5ccccc54)c3)cc2)c2ccc(-c3ccc4c(ccc5ccccc54)c3)cc2)c1 ; c1cc(-c2cccc3c2ccc2ccccc23)cc(N(c2ccc(-c3ccc4ccccc4c3)cc2)c2ccc(-c3ccc4c(ccc5ccccc54)c3)cc2)c1 ; c1ccc(-c2ccc(N(c3ccc(-c4ccc5c(ccc6ccccc65)c4)cc3)c3cccc(-c4cccc5c4ccc4ccccc45)c3)c3ccccc23)cc1]

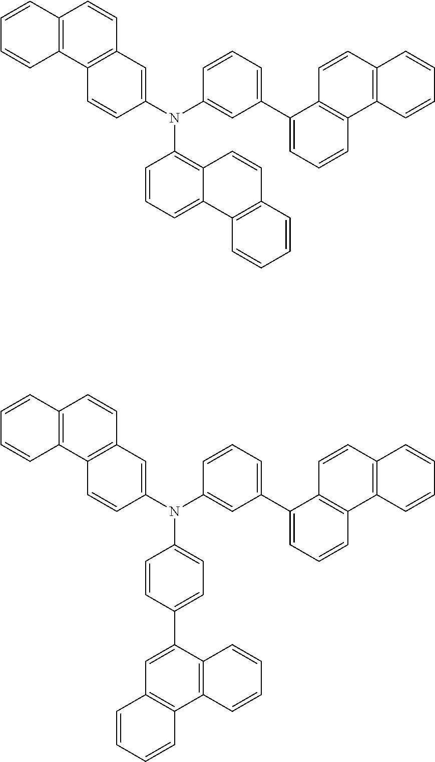 [SMILES: c1cc(-c2cccc3c2ccc2ccccc23)cc(N(c2ccc3c(ccc4ccccc43)c2)c2cccc3c2ccc2ccccc23)c1 ; c1cc(-c2cccc3c2ccc2ccccc23)cc(N(c2ccc(-c3cc4ccccc4c4ccccc34)cc2)c2ccc3c(ccc4ccccc43)c2)c1]

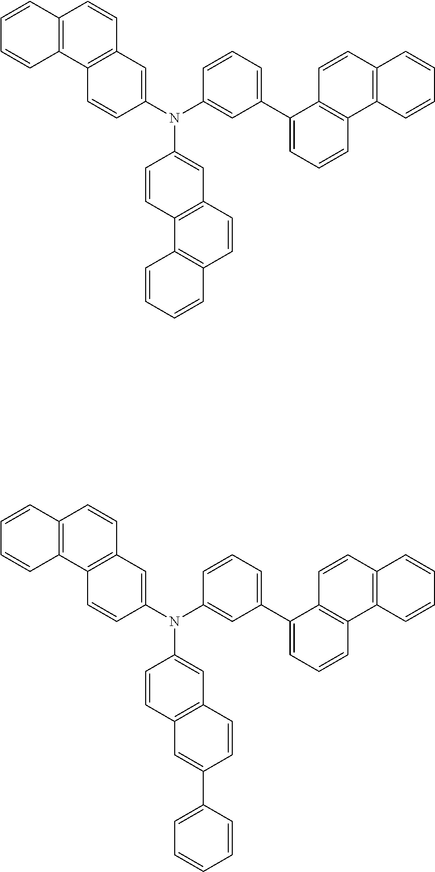 [SMILES: c1cc(-c2cccc3c2ccc2ccccc23)cc(N(c2ccc3c(ccc4ccccc43)c2)c2ccc3c(ccc4ccccc43)c2)c1 ; c1ccc(-c2ccc3cc(N(c4cccc(-c5cccc6c5ccc5ccccc56)c4)c4ccc5c(ccc6ccccc65)c4)ccc3c2)cc1]

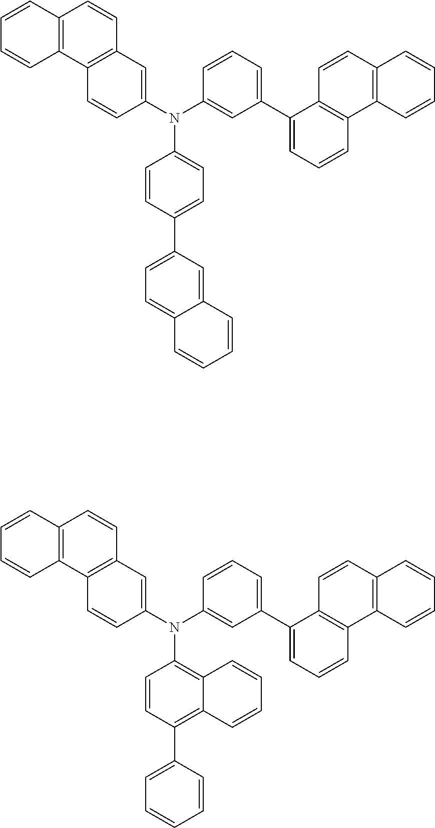 [SMILES: c1cc(-c2cccc3c2ccc2ccccc23)cc(N(c2ccc(-c3ccc4ccccc4c3)cc2)c2ccc3c(ccc4ccccc43)c2)c1 ; c1ccc(-c2ccc(N(c3cccc(-c4cccc5c4ccc4ccccc45)c3)c3ccc4c(ccc5ccccc54)c3)c3ccccc23)cc1]

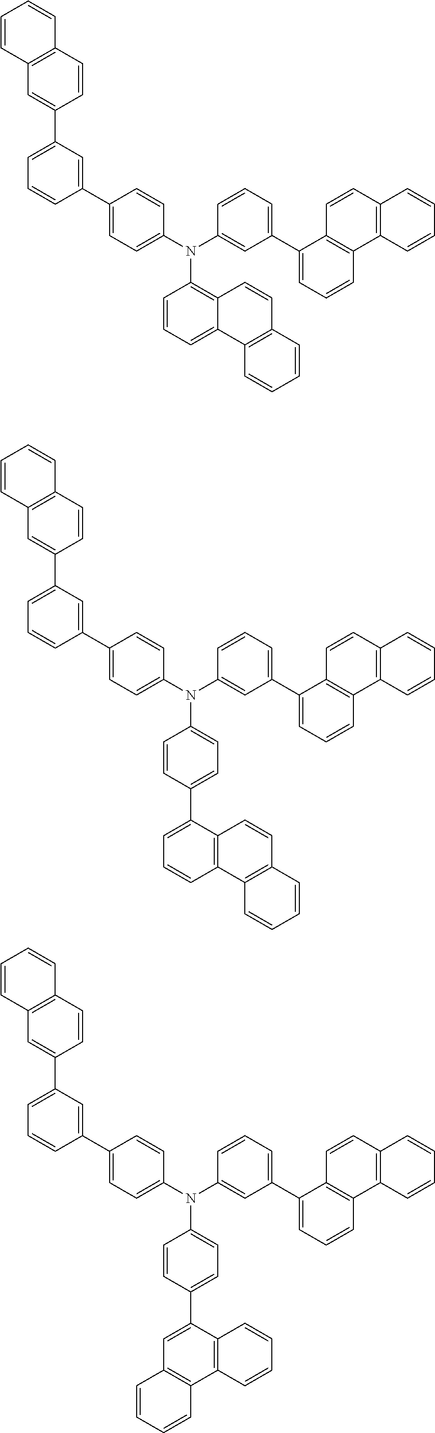 [SMILES: c1cc(-c2ccc(N(c3cccc(-c4cccc5c4ccc4ccccc45)c3)c3cccc4c3ccc3ccccc34)cc2)cc(-c2ccc3ccccc3c2)c1 ; c1cc(-c2ccc(N(c3ccc(-c4cccc5c4ccc4ccccc45)cc3)c3cccc(-c4cccc5c4ccc4ccccc45)c3)cc2)cc(-c2ccc3ccccc3c2)c1 ; c1cc(-c2ccc(N(c3ccc(-c4cc5ccccc5c5ccccc45)cc3)c3cccc(-c4cccc5c4ccc4ccccc45)c3)cc2)cc(-c2ccc3ccccc3c2)c1]

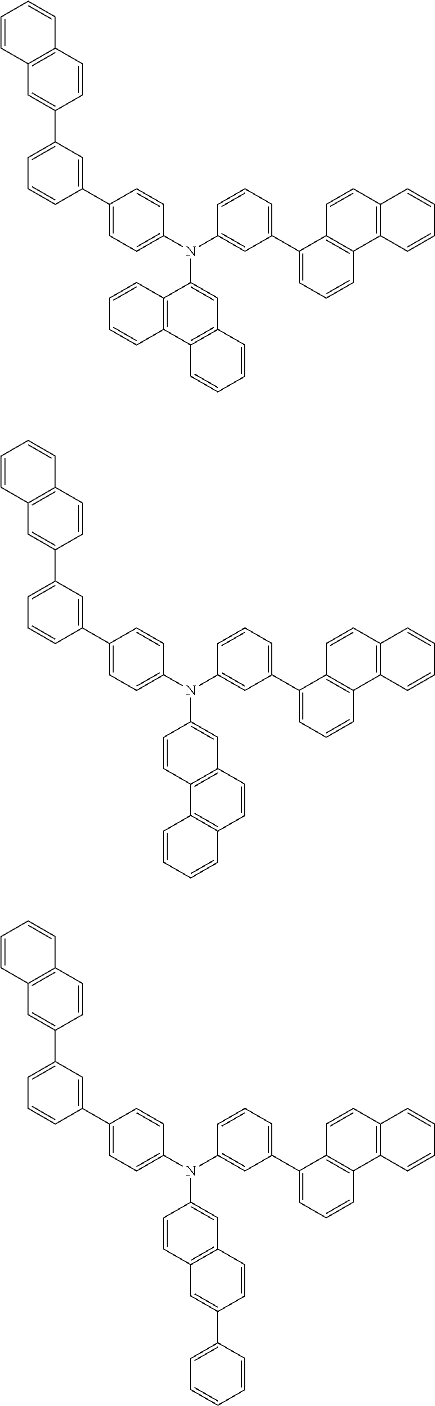 [SMILES: c1cc(-c2ccc(N(c3cccc(-c4cccc5c4ccc4ccccc45)c3)c3cc4ccccc4c4ccccc34)cc2)cc(-c2ccc3ccccc3c2)c1 ; c1cc(-c2ccc(N(c3cccc(-c4cccc5c4ccc4ccccc45)c3)c3ccc4c(ccc5ccccc54)c3)cc2)cc(-c2ccc3ccccc3c2)c1 ; c1ccc(-c2ccc3cc(N(c4ccc(-c5cccc(-c6ccc7ccccc7c6)c5)cc4)c4cccc(-c5cccc6c5ccc5ccccc56)c4)ccc3c2)cc1]

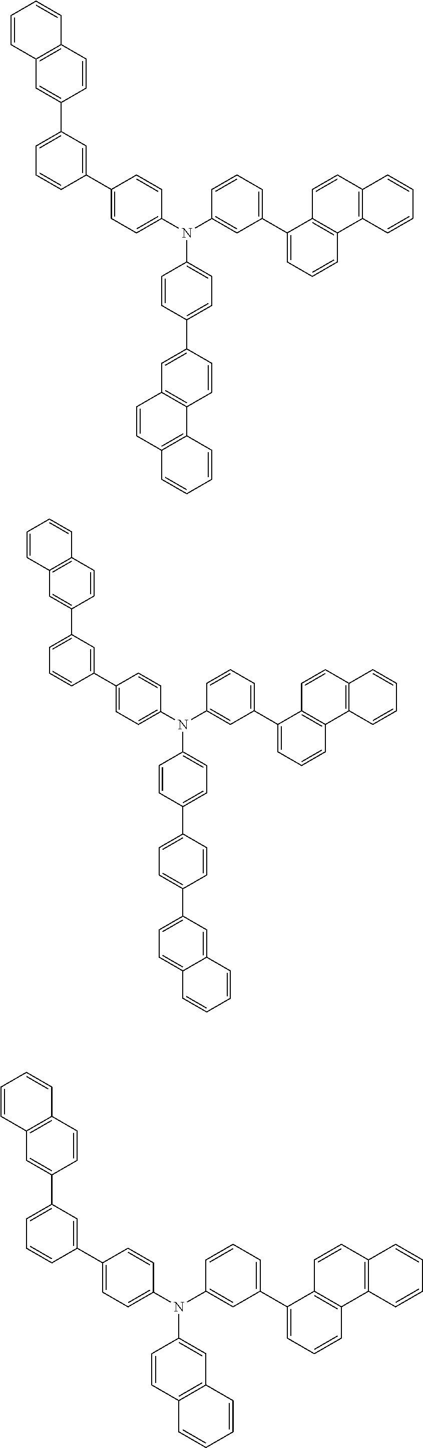 [SMILES: c1cc(-c2ccc(N(c3ccc(-c4ccc5c(ccc6ccccc65)c4)cc3)c3cccc(-c4cccc5c4ccc4ccccc45)c3)cc2)cc(-c2ccc3ccccc3c2)c1 ; c1cc(-c2ccc(N(c3ccc(-c4ccc(-c5ccc6ccccc6c5)cc4)cc3)c3cccc(-c4cccc5c4ccc4ccccc45)c3)cc2)cc(-c2ccc3ccccc3c2)c1 ; c1cc(-c2ccc(N(c3cccc(-c4cccc5c4ccc4ccccc45)c3)c3ccc4ccccc4c3)cc2)cc(-c2ccc3ccccc3c2)c1]

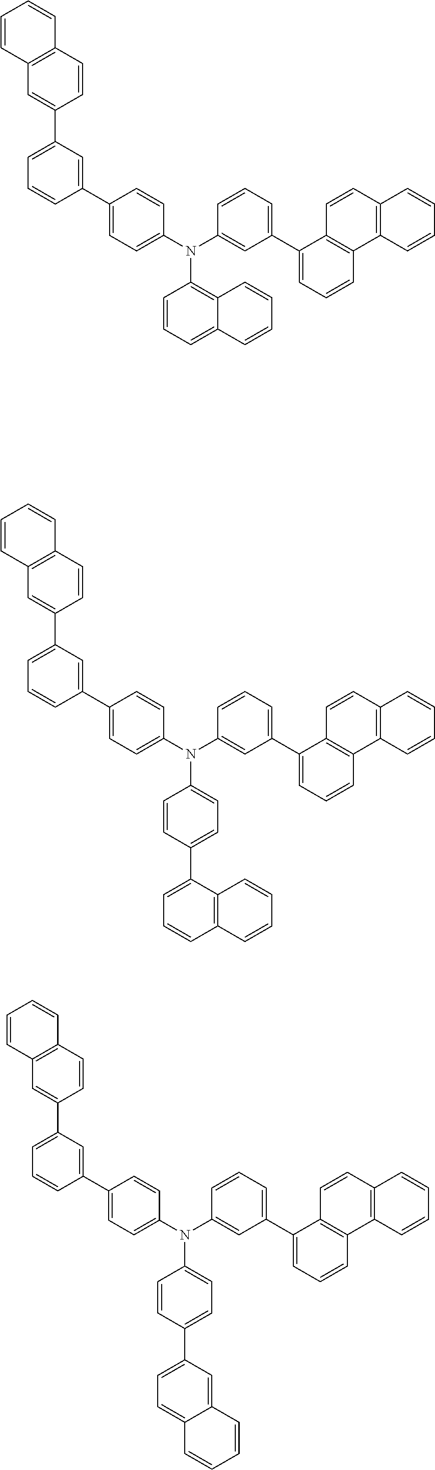 [SMILES: c1cc(-c2ccc(N(c3cccc(-c4cccc5c4ccc4ccccc45)c3)c3cccc4ccccc34)cc2)cc(-c2ccc3ccccc3c2)c1 ; c1cc(-c2ccc(N(c3ccc(-c4cccc5ccccc45)cc3)c3cccc(-c4cccc5c4ccc4ccccc45)c3)cc2)cc(-c2ccc3ccccc3c2)c1 ; c1cc(-c2ccc(N(c3ccc(-c4ccc5ccccc5c4)cc3)c3cccc(-c4cccc5c4ccc4ccccc45)c3)cc2)cc(-c2ccc3ccccc3c2)c1]

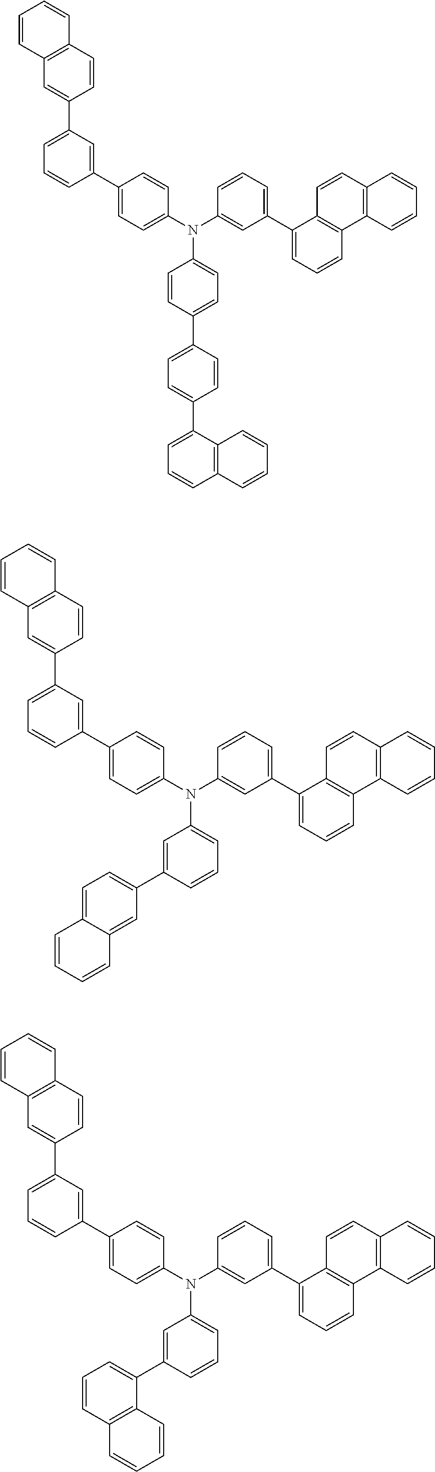 [SMILES: c1cc(-c2ccc(N(c3ccc(-c4ccc(-c5cccc6ccccc56)cc4)cc3)c3cccc(-c4cccc5c4ccc4ccccc45)c3)cc2)cc(-c2ccc3ccccc3c2)c1 ; c1cc(-c2ccc(N(c3cccc(-c4ccc5ccccc5c4)c3)c3cccc(-c4cccc5c4ccc4ccccc45)c3)cc2)cc(-c2ccc3ccccc3c2)c1 ; c1cc(-c2ccc(N(c3cccc(-c4cccc5ccccc45)c3)c3cccc(-c4cccc5c4ccc4ccccc45)c3)cc2)cc(-c2ccc3ccccc3c2)c1]

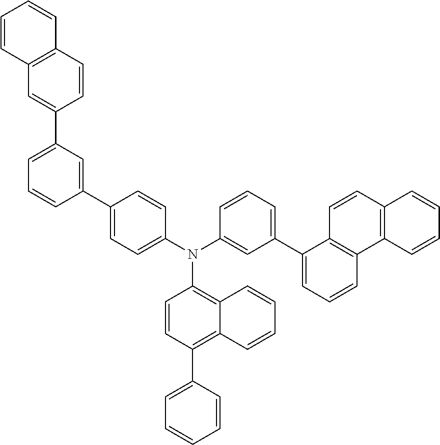 [SMILES: c1ccc(-c2ccc(N(c3ccc(-c4cccc(-c5ccc6ccccc6c5)c4)cc3)c3cccc(-c4cccc5c4ccc4ccccc45)c3)c3ccccc23)cc1]

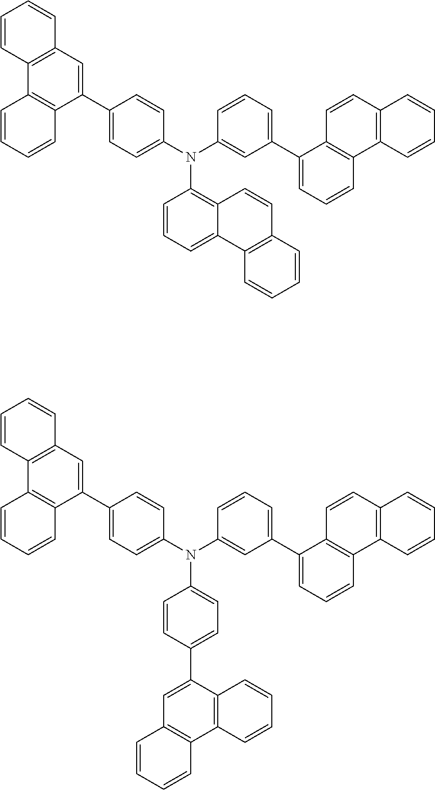 [SMILES: c1cc(-c2cccc3c2ccc2ccccc23)cc(N(c2ccc(-c3cc4ccccc4c4ccccc34)cc2)c2cccc3c2ccc2ccccc23)c1 ; c1cc(-c2cccc3c2ccc2ccccc23)cc(N(c2ccc(-c3cc4ccccc4c4ccccc34)cc2)c2ccc(-c3cc4ccccc4c4ccccc34)cc2)c1]

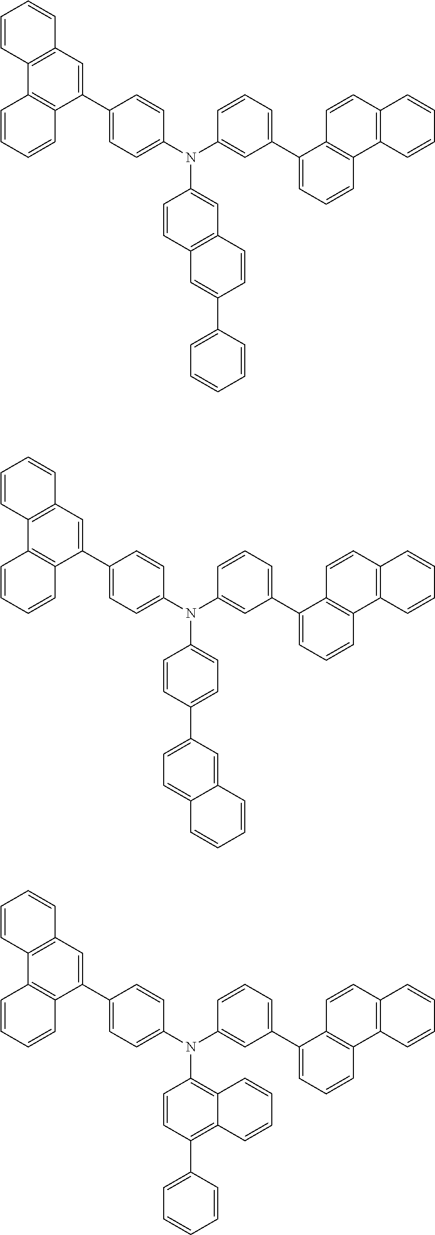 [SMILES: c1ccc(-c2ccc3cc(N(c4ccc(-c5cc6ccccc6c6ccccc56)cc4)c4cccc(-c5cccc6c5ccc5ccccc56)c4)ccc3c2)cc1 ; c1cc(-c2cccc3c2ccc2ccccc23)cc(N(c2ccc(-c3ccc4ccccc4c3)cc2)c2ccc(-c3cc4ccccc4c4ccccc34)cc2)c1 ; c1ccc(-c2ccc(N(c3ccc(-c4cc5ccccc5c5ccccc45)cc3)c3cccc(-c4cccc5c4ccc4ccccc45)c3)c3ccccc23)cc1]

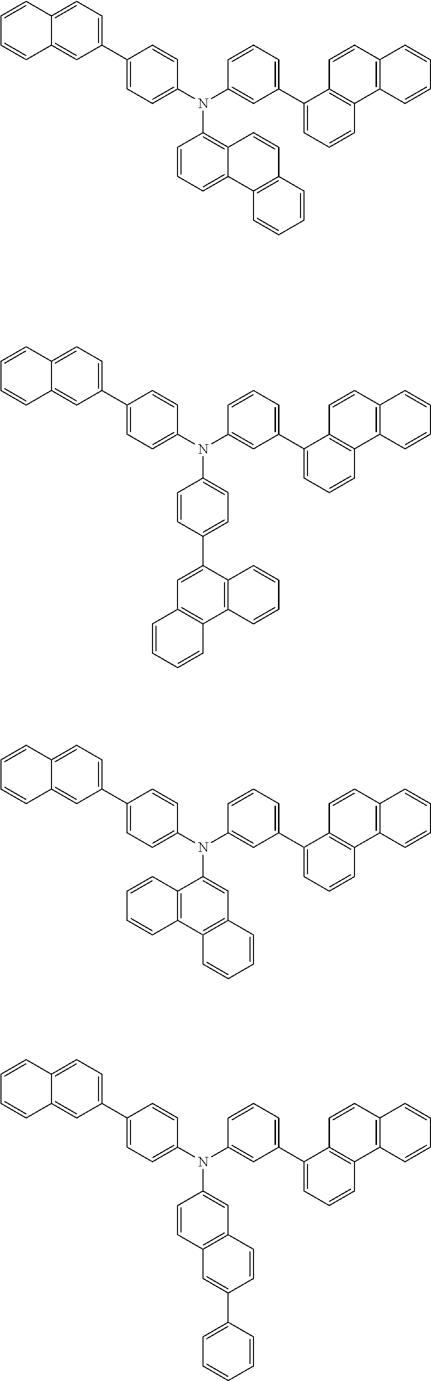 [SMILES: c1cc(-c2cccc3c2ccc2ccccc23)cc(N(c2ccc(-c3ccc4ccccc4c3)cc2)c2cccc3c2ccc2ccccc23)c1 ; c1cc(-c2cccc3c2ccc2ccccc23)cc(N(c2ccc(-c3ccc4ccccc4c3)cc2)c2ccc(-c3cc4ccccc4c4ccccc34)cc2)c1 ; c1cc(-c2cccc3c2ccc2ccccc23)cc(N(c2ccc(-c3ccc4ccccc4c3)cc2)c2cc3ccccc3c3ccccc23)c1 ; c1ccc(-c2ccc3cc(N(c4ccc(-c5ccc6ccccc6c5)cc4)c4cccc(-c5cccc6c5ccc5ccccc56)c4)ccc3c2)cc1]

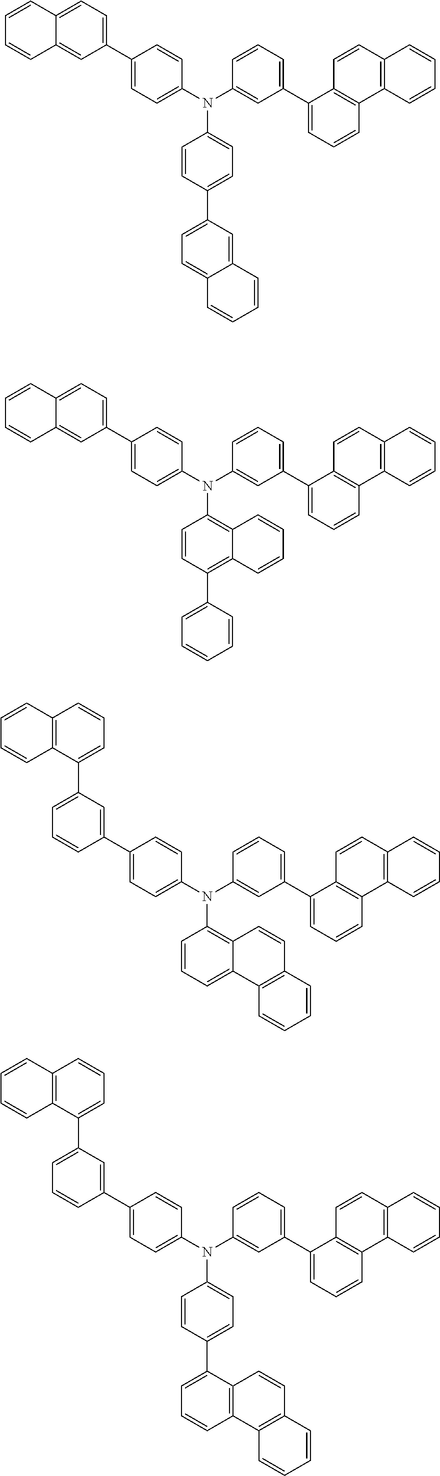 [SMILES: c1cc(-c2cccc3c2ccc2ccccc23)cc(N(c2ccc(-c3ccc4ccccc4c3)cc2)c2ccc(-c3ccc4ccccc4c3)cc2)c1 ; c1ccc(-c2ccc(N(c3ccc(-c4ccc5ccccc5c4)cc3)c3cccc(-c4cccc5c4ccc4ccccc45)c3)c3ccccc23)cc1 ; c1cc(-c2ccc(N(c3cccc(-c4cccc5c4ccc4ccccc45)c3)c3cccc4c3ccc3ccccc34)cc2)cc(-c2cccc3ccccc23)c1 ; c1cc(-c2ccc(N(c3ccc(-c4cccc5c4ccc4ccccc45)cc3)c3cccc(-c4cccc5c4ccc4ccccc45)c3)cc2)cc(-c2cccc3ccccc23)c1]

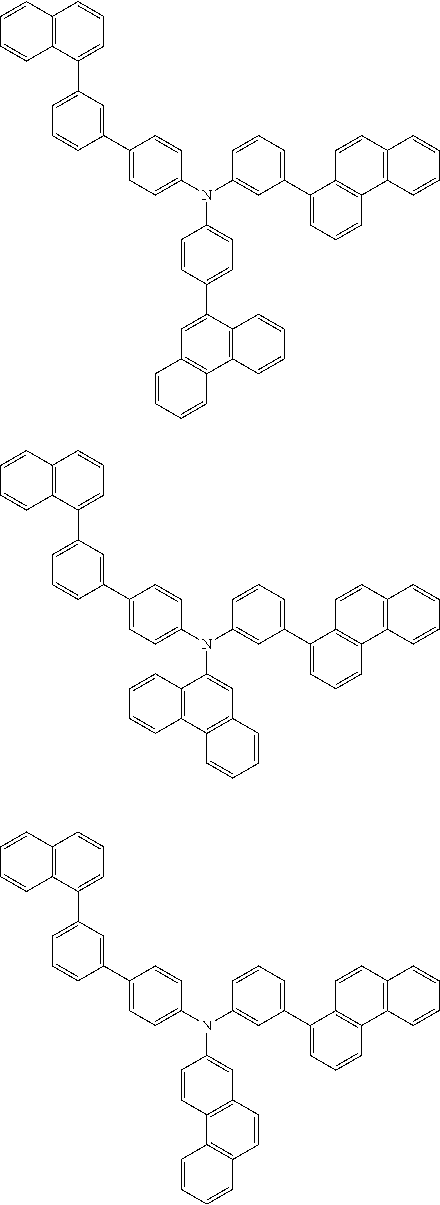 [SMILES: c1cc(-c2ccc(N(c3ccc(-c4cc5ccccc5c5ccccc45)cc3)c3cccc(-c4cccc5c4ccc4ccccc45)c3)cc2)cc(-c2cccc3ccccc23)c1 ; c1cc(-c2ccc(N(c3cccc(-c4cccc5c4ccc4ccccc45)c3)c3cc4ccccc4c4ccccc34)cc2)cc(-c2cccc3ccccc23)c1 ; c1cc(-c2ccc(N(c3cccc(-c4cccc5c4ccc4ccccc45)c3)c3ccc4c(ccc5ccccc54)c3)cc2)cc(-c2cccc3ccccc23)c1]

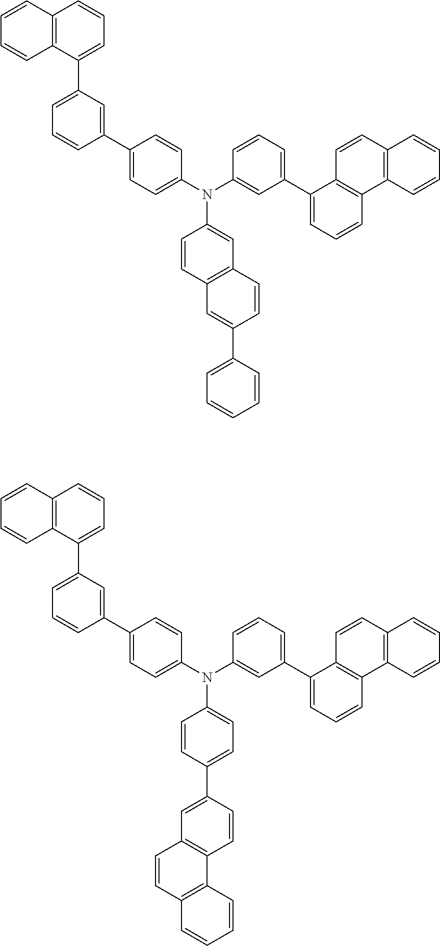 [SMILES: c1ccc(-c2ccc3cc(N(c4ccc(-c5cccc(-c6cccc7ccccc67)c5)cc4)c4cccc(-c5cccc6c5ccc5ccccc56)c4)ccc3c2)cc1 ; c1cc(-c2ccc(N(c3ccc(-c4ccc5c(ccc6ccccc65)c4)cc3)c3cccc(-c4cccc5c4ccc4ccccc45)c3)cc2)cc(-c2cccc3ccccc23)c1]

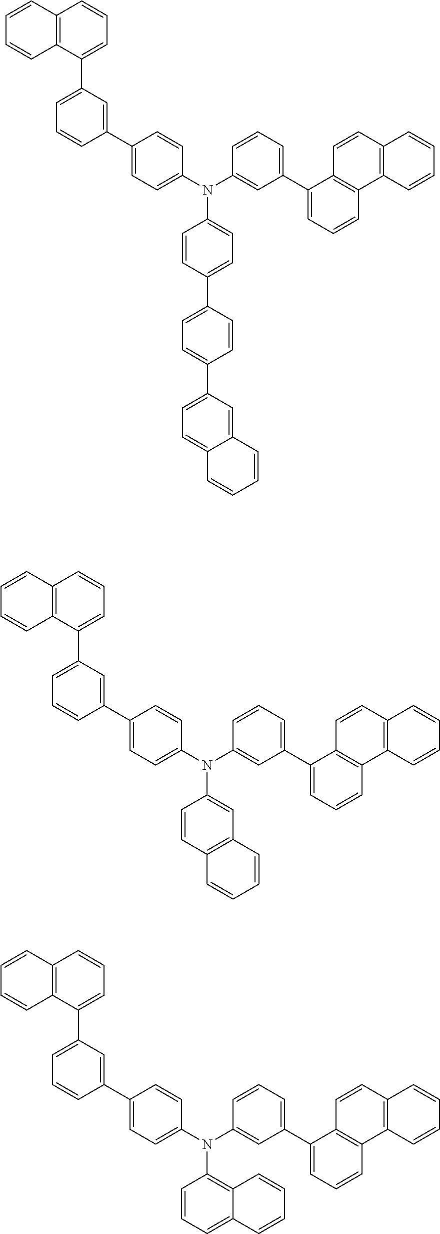 [SMILES: c1cc(-c2ccc(N(c3ccc(-c4ccc(-c5ccc6ccccc6c5)cc4)cc3)c3cccc(-c4cccc5c4ccc4ccccc45)c3)cc2)cc(-c2cccc3ccccc23)c1 ; c1cc(-c2ccc(N(c3cccc(-c4cccc5c4ccc4ccccc45)c3)c3ccc4ccccc4c3)cc2)cc(-c2cccc3ccccc23)c1 ; c1cc(-c2ccc(N(c3cccc(-c4cccc5c4ccc4ccccc45)c3)c3cccc4ccccc34)cc2)cc(-c2cccc3ccccc23)c1]

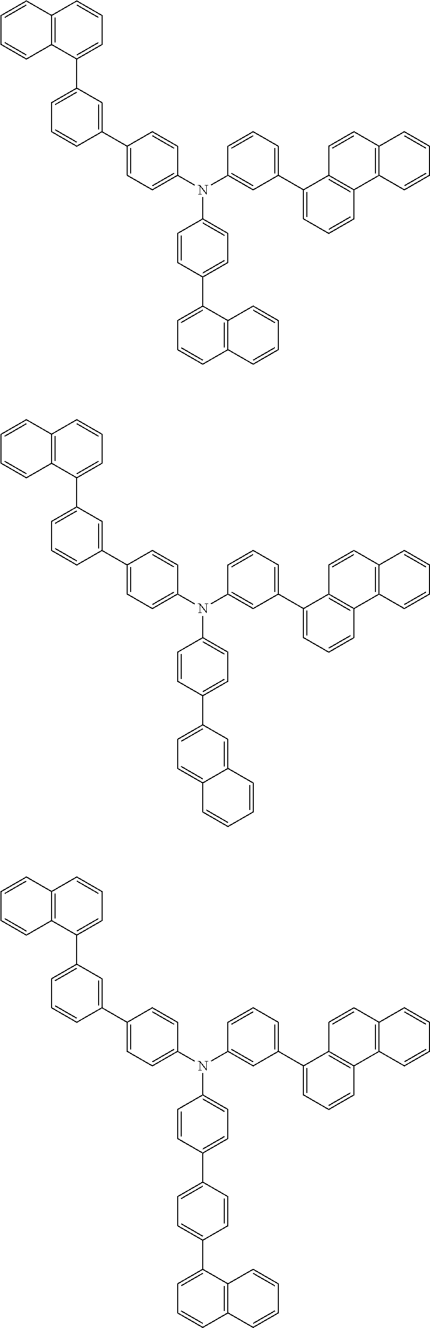 [SMILES: c1cc(-c2ccc(N(c3ccc(-c4cccc5ccccc45)cc3)c3cccc(-c4cccc5c4ccc4ccccc45)c3)cc2)cc(-c2cccc3ccccc23)c1 ; c1cc(-c2ccc(N(c3ccc(-c4ccc5ccccc5c4)cc3)c3cccc(-c4cccc5c4ccc4ccccc45)c3)cc2)cc(-c2cccc3ccccc23)c1 ; c1cc(-c2ccc(N(c3ccc(-c4ccc(-c5cccc6ccccc56)cc4)cc3)c3cccc(-c4cccc5c4ccc4ccccc45)c3)cc2)cc(-c2cccc3ccccc23)c1]

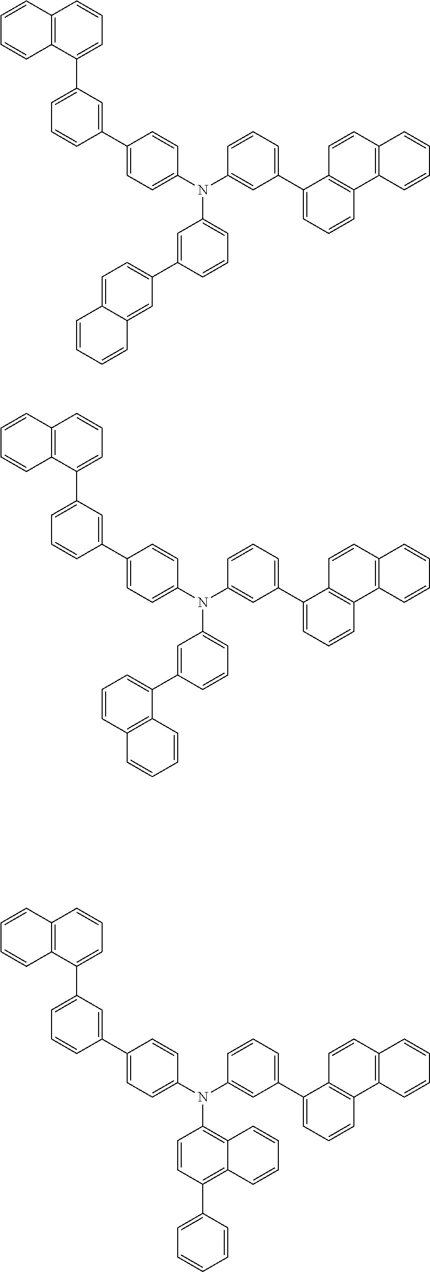 [SMILES: c1cc(-c2ccc(N(c3cccc(-c4ccc5ccccc5c4)c3)c3cccc(-c4cccc5c4ccc4ccccc45)c3)cc2)cc(-c2cccc3ccccc23)c1 ; c1cc(-c2ccc(N(c3cccc(-c4cccc5ccccc45)c3)c3cccc(-c4cccc5c4ccc4ccccc45)c3)cc2)cc(-c2cccc3ccccc23)c1 ; c1ccc(-c2ccc(N(c3ccc(-c4cccc(-c5cccc6ccccc56)c4)cc3)c3cccc(-c4cccc5c4ccc4ccccc45)c3)c3ccccc23)cc1]

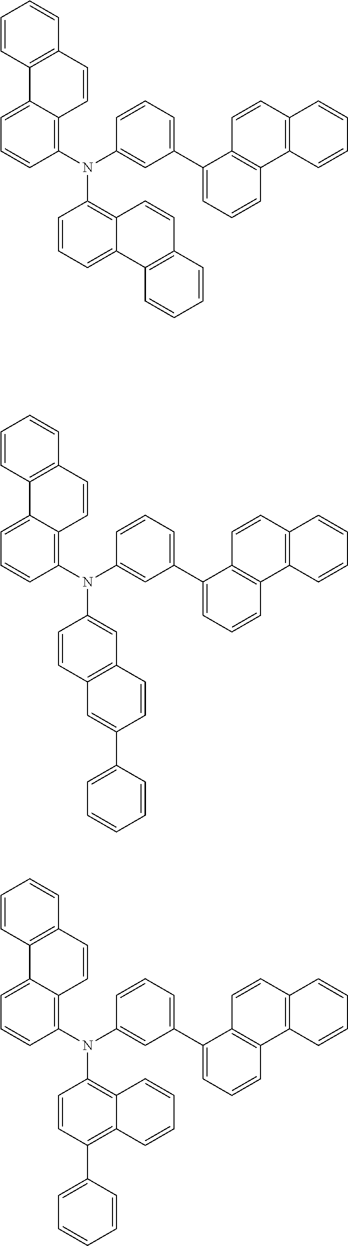 [SMILES: c1cc(-c2cccc3c2ccc2ccccc23)cc(N(c2cccc3c2ccc2ccccc23)c2cccc3c2ccc2ccccc23)c1 ; c1ccc(-c2ccc3cc(N(c4cccc(-c5cccc6c5ccc5ccccc56)c4)c4cccc5c4ccc4ccccc45)ccc3c2)cc1 ; c1ccc(-c2ccc(N(c3cccc(-c4cccc5c4ccc4ccccc45)c3)c3cccc4c3ccc3ccccc34)c3ccccc23)cc1]

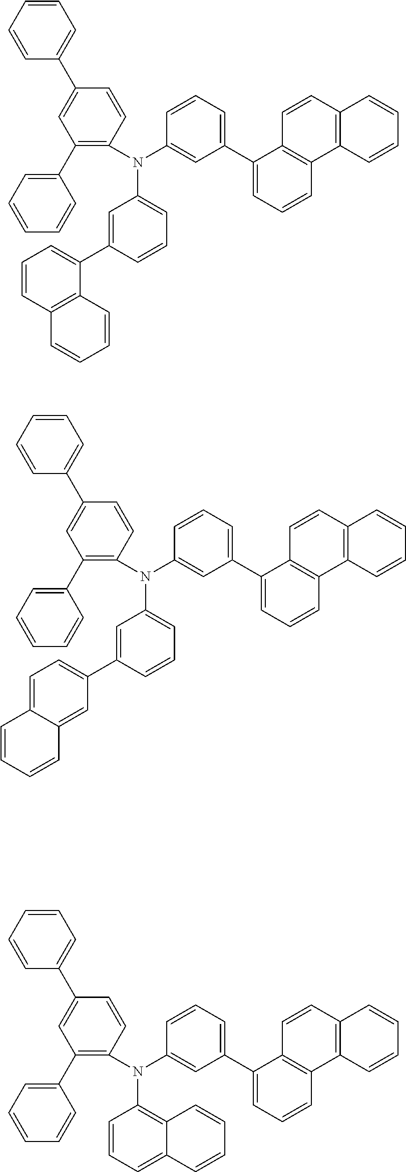 [SMILES: c1ccc(-c2ccc(N(c3cccc(-c4cccc5ccccc45)c3)c3cccc(-c4cccc5c4ccc4ccccc45)c3)c(-c3ccccc3)c2)cc1 ; c1ccc(-c2ccc(N(c3cccc(-c4ccc5ccccc5c4)c3)c3cccc(-c4cccc5c4ccc4ccccc45)c3)c(-c3ccccc3)c2)cc1 ; c1ccc(-c2ccc(N(c3cccc(-c4cccc5c4ccc4ccccc45)c3)c3cccc4ccccc34)c(-c3ccccc3)c2)cc1]

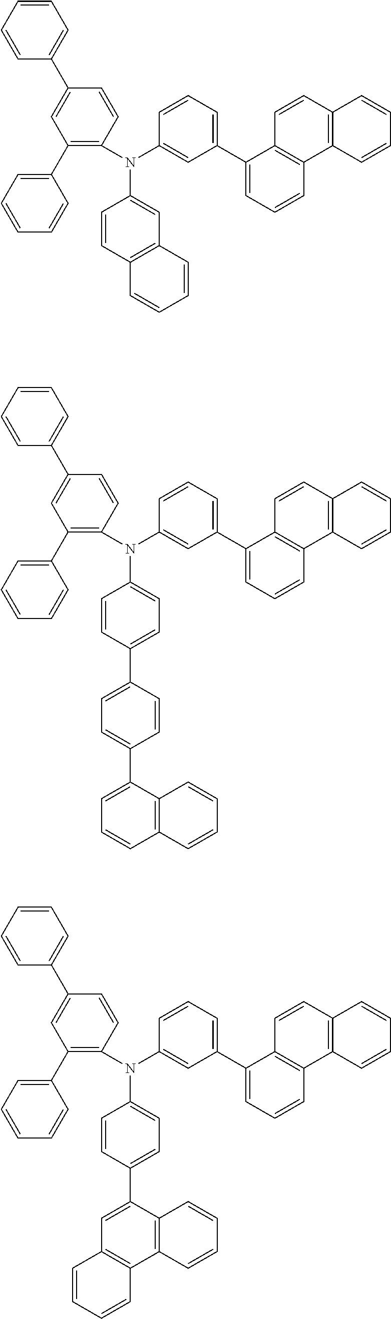 [SMILES: c1ccc(-c2ccc(N(c3cccc(-c4cccc5c4ccc4ccccc45)c3)c3ccc4ccccc4c3)c(-c3ccccc3)c2)cc1 ; c1ccc(-c2ccc(N(c3ccc(-c4ccc(-c5cccc6ccccc56)cc4)cc3)c3cccc(-c4cccc5c4ccc4ccccc45)c3)c(-c3ccccc3)c2)cc1 ; c1ccc(-c2ccc(N(c3ccc(-c4cc5ccccc5c5ccccc45)cc3)c3cccc(-c4cccc5c4ccc4ccccc45)c3)c(-c3ccccc3)c2)cc1]

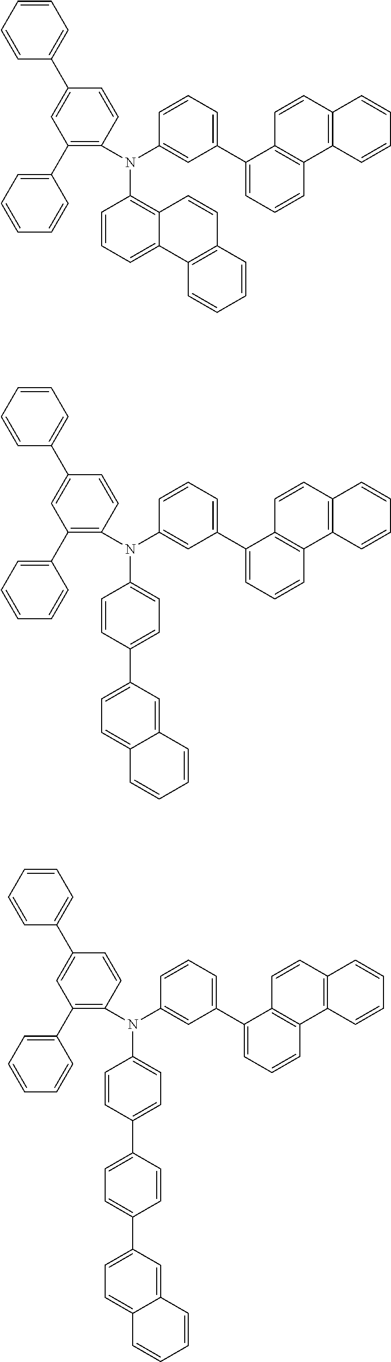 [SMILES: c1ccc(-c2ccc(N(c3cccc(-c4cccc5c4ccc4ccccc45)c3)c3cccc4c3ccc3ccccc34)c(-c3ccccc3)c2)cc1 ; c1ccc(-c2ccc(N(c3ccc(-c4ccc5ccccc5c4)cc3)c3cccc(-c4cccc5c4ccc4ccccc45)c3)c(-c3ccccc3)c2)cc1 ; c1ccc(-c2ccc(N(c3ccc(-c4ccc(-c5ccc6ccccc6c5)cc4)cc3)c3cccc(-c4cccc5c4ccc4ccccc45)c3)c(-c3ccccc3)c2)cc1]

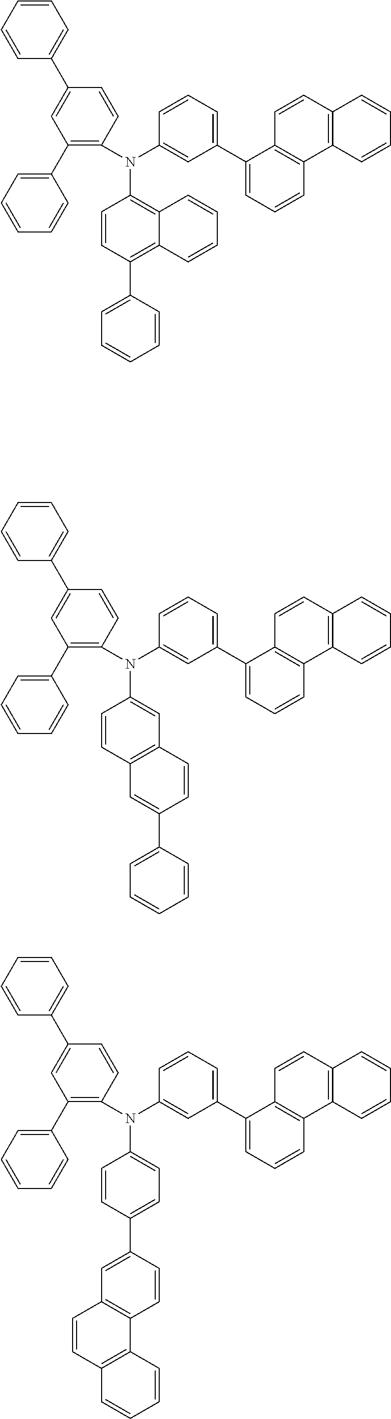 [SMILES: c1ccc(-c2ccc(N(c3cccc(-c4cccc5c4ccc4ccccc45)c3)c3ccc(-c4ccccc4)c4ccccc34)c(-c3ccccc3)c2)cc1 ; c1ccc(-c2ccc(N(c3cccc(-c4cccc5c4ccc4ccccc45)c3)c3ccc4cc(-c5ccccc5)ccc4c3)c(-c3ccccc3)c2)cc1 ; c1ccc(-c2ccc(N(c3ccc(-c4ccc5c(ccc6ccccc65)c4)cc3)c3cccc(-c4cccc5c4ccc4ccccc45)c3)c(-c3ccccc3)c2)cc1]

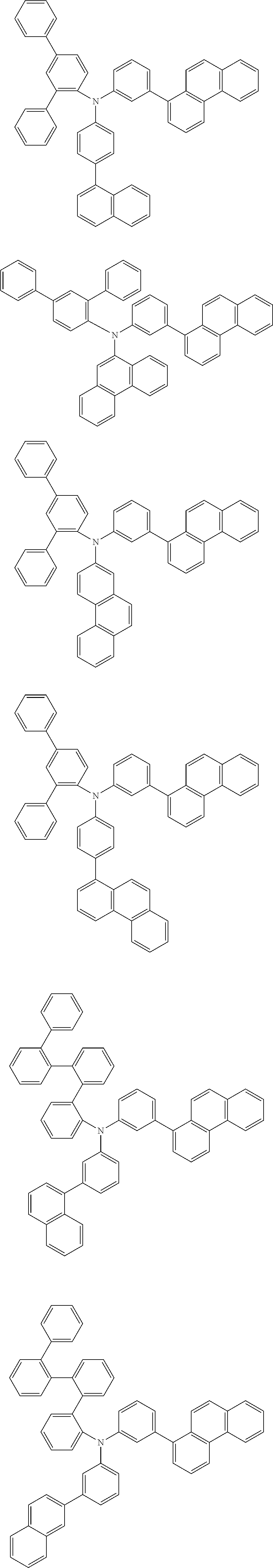

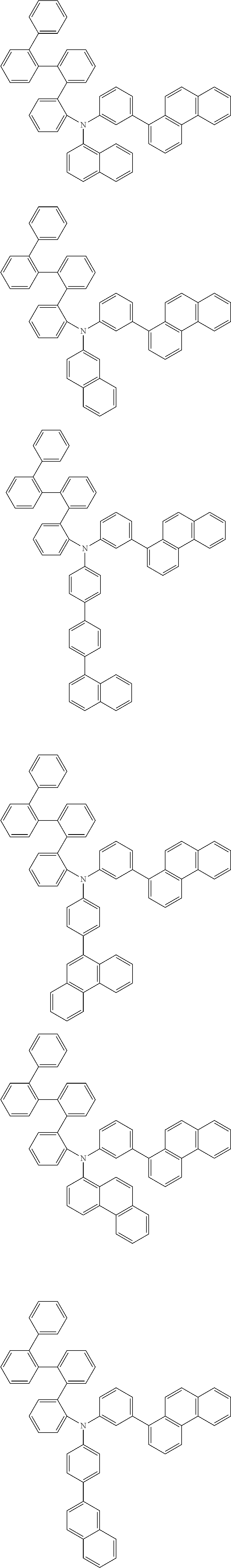

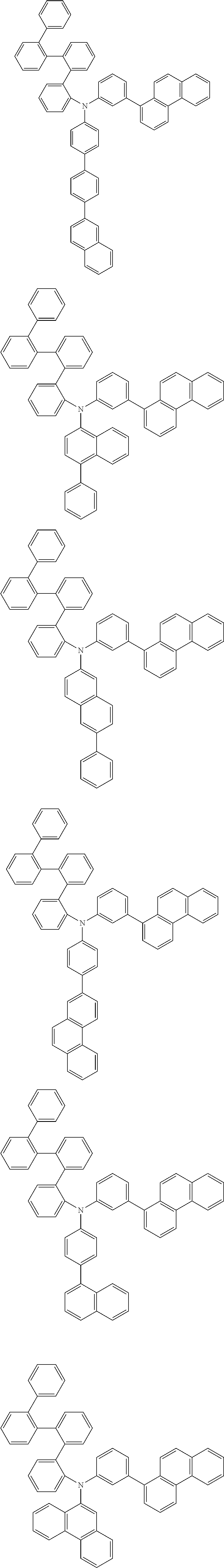

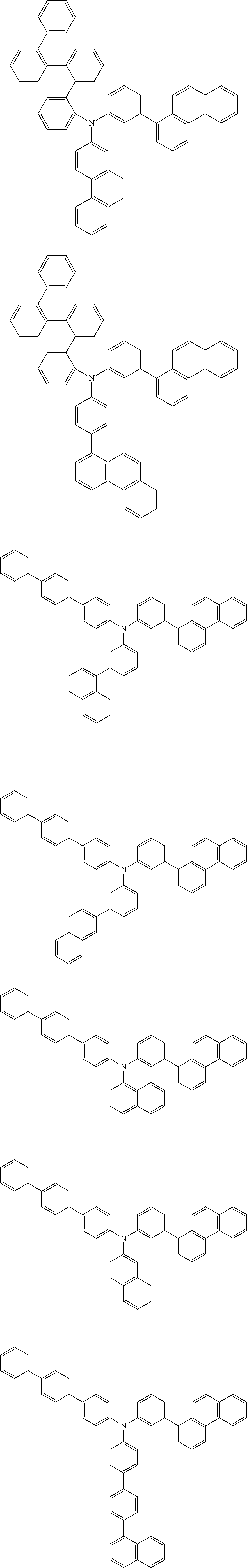

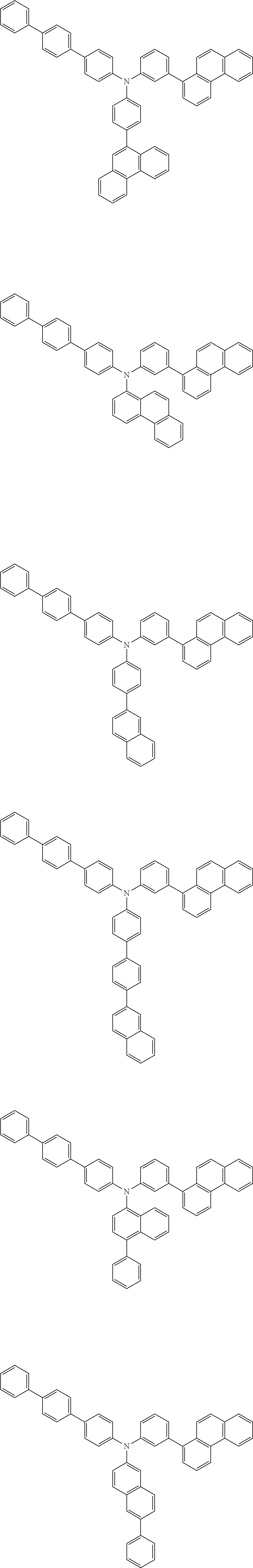

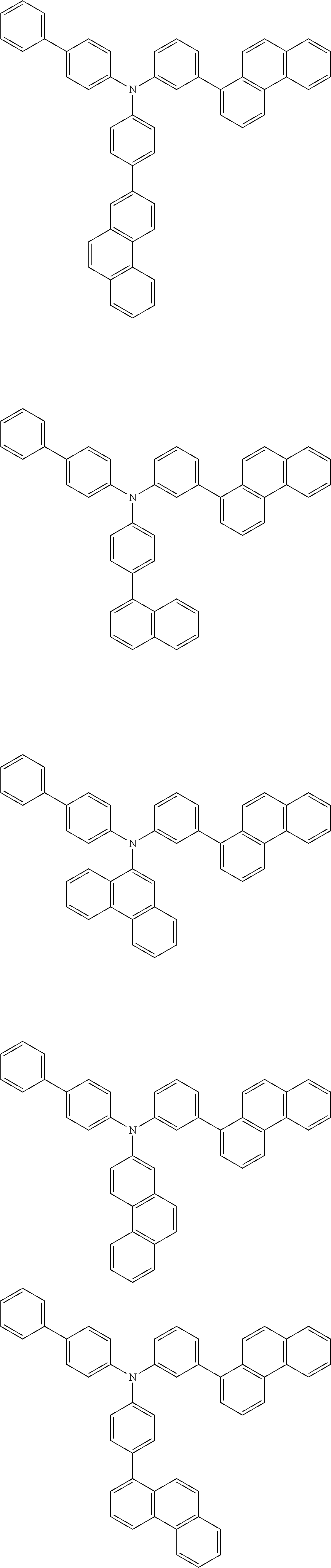 [SMILES: c1ccc(-c2ccc(N(c3ccc(-c4ccc5c(ccc6ccccc65)c4)cc3)c3cccc(-c4cccc5c4ccc4ccccc45)c3)cc2)cc1 ; c1ccc(-c2ccc(N(c3ccc(-c4cccc5ccccc45)cc3)c3cccc(-c4cccc5c4ccc4ccccc45)c3)cc2)cc1 ; c1ccc(-c2ccc(N(c3cccc(-c4cccc5c4ccc4ccccc45)c3)c3cc4ccccc4c4ccccc34)cc2)cc1 ; c1ccc(-c2ccc(N(c3cccc(-c4cccc5c4ccc4ccccc45)c3)c3ccc4c(ccc5ccccc54)c3)cc2)cc1 ; c1ccc(-c2ccc(N(c3ccc(-c4cccc5c4ccc4ccccc45)cc3)c3cccc(-c4cccc5c4ccc4ccccc45)c3)cc2)cc1]

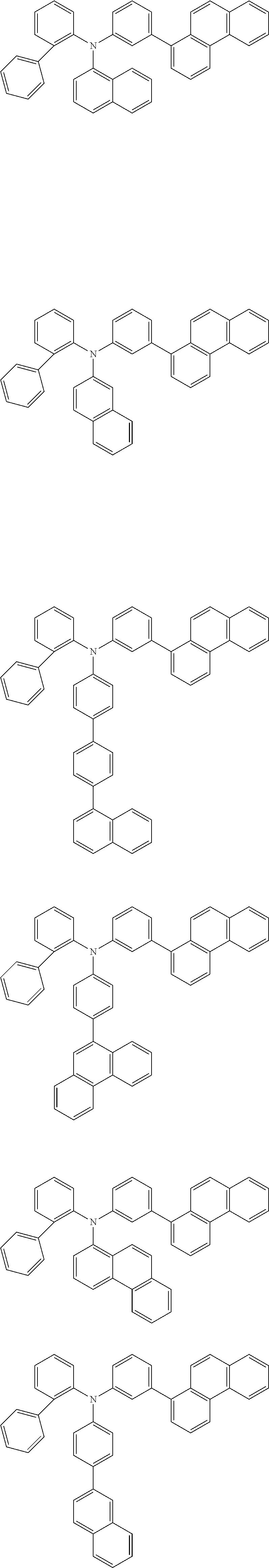 [SMILES: c1ccc(-c2ccccc2N(c2cccc(-c3cccc4c3ccc3ccccc34)c2)c2cccc3ccccc23)cc1 ; c1ccc(-c2ccccc2N(c2cccc(-c3cccc4c3ccc3ccccc34)c2)c2ccc3ccccc3c2)cc1 ; c1ccc(-c2ccccc2N(c2ccc(-c3ccc(-c4cccc5ccccc45)cc3)cc2)c2cccc(-c3cccc4c3ccc3ccccc34)c2)cc1 ; c1ccc(-c2ccccc2N(c2ccc(-c3cc4ccccc4c4ccccc34)cc2)c2cccc(-c3cccc4c3ccc3ccccc34)c2)cc1 ; c1ccc(-c2ccccc2N(c2cccc(-c3cccc4c3ccc3ccccc34)c2)c2cccc3c2ccc2ccccc23)cc1 ; c1ccc(-c2ccccc2N(c2ccc(-c3ccc4ccccc4c3)cc2)c2cccc(-c3cccc4c3ccc3ccccc34)c2)cc1]

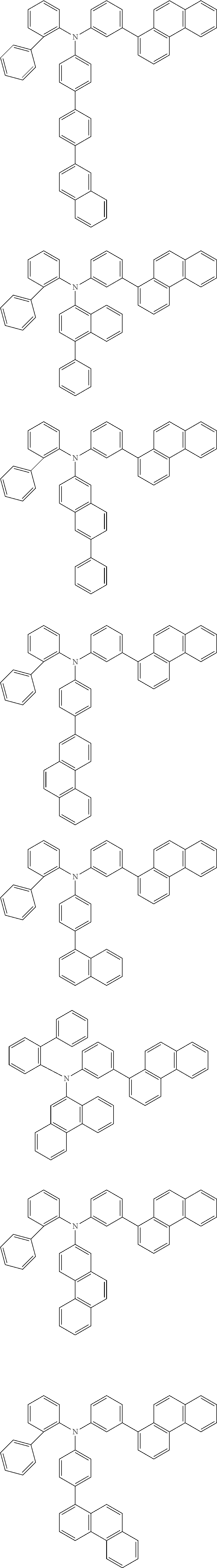

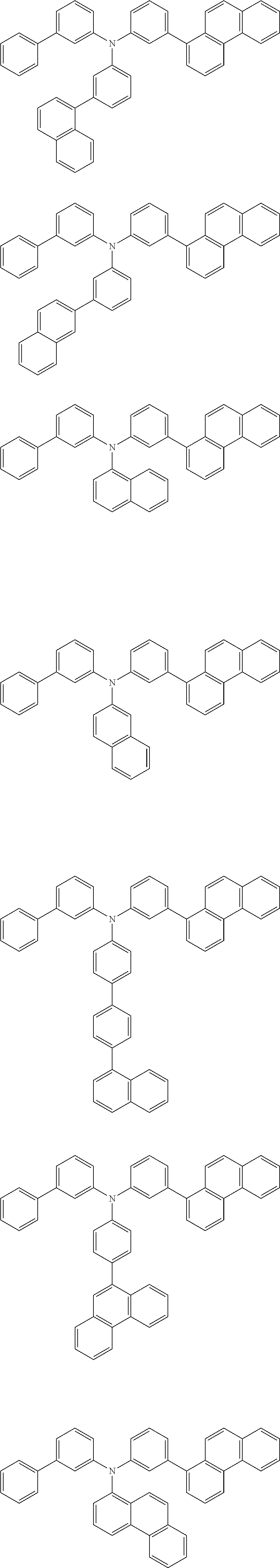

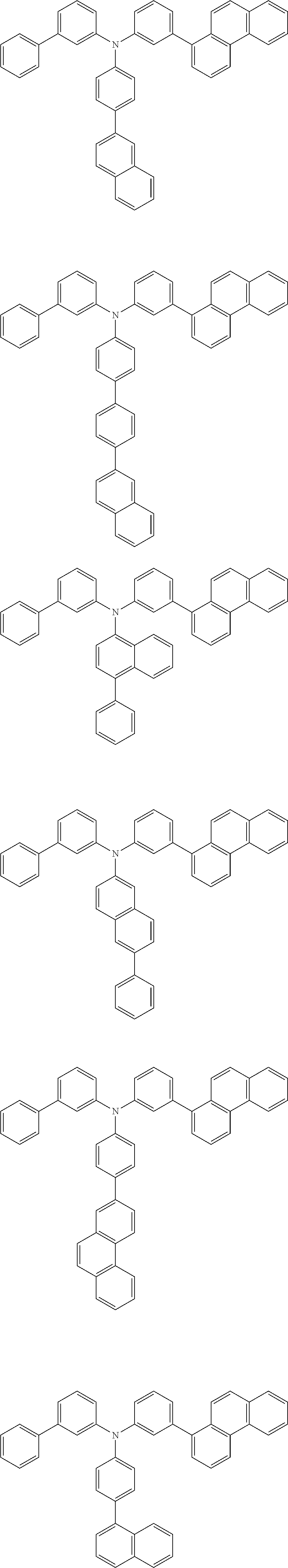

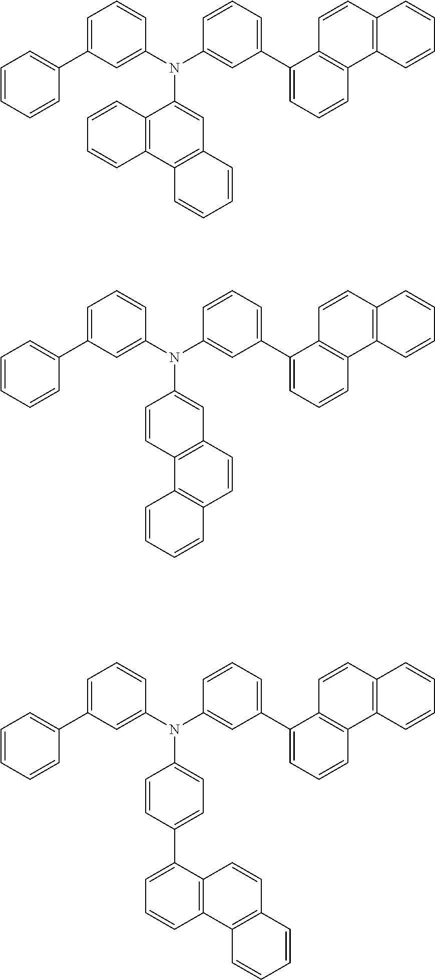 [SMILES: c1ccc(-c2cccc(N(c3cccc(-c4cccc5c4ccc4ccccc45)c3)c3cc4ccccc4c4ccccc34)c2)cc1 ; c1ccc(-c2cccc(N(c3cccc(-c4cccc5c4ccc4ccccc45)c3)c3ccc4c(ccc5ccccc54)c3)c2)cc1 ; c1ccc(-c2cccc(N(c3ccc(-c4cccc5c4ccc4ccccc45)cc3)c3cccc(-c4cccc5c4ccc4ccccc45)c3)c2)cc1]

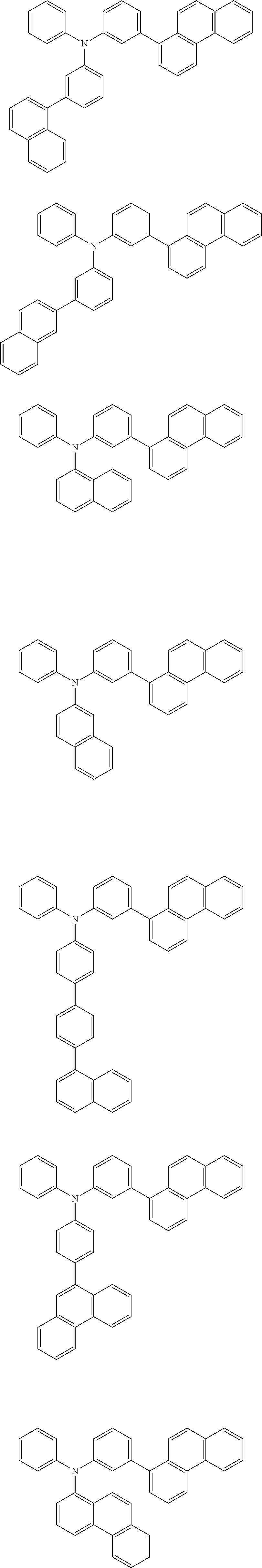

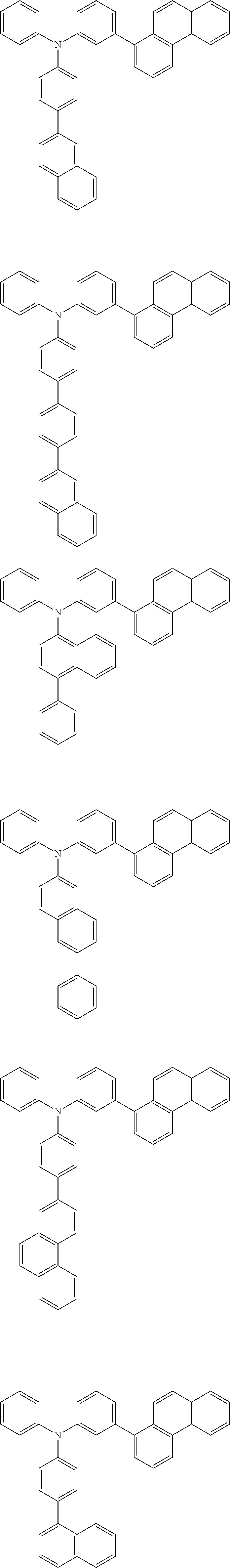

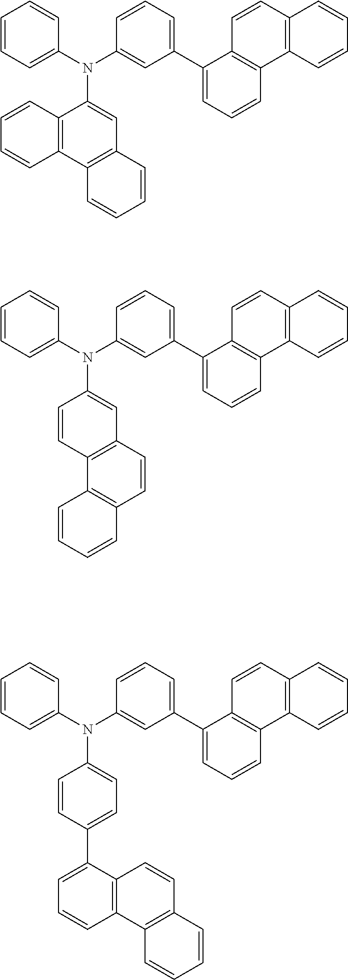 [SMILES: c1ccc(N(c2cccc(-c3cccc4c3ccc3ccccc34)c2)c2cc3ccccc3c3ccccc23)cc1 ; c1ccc(N(c2cccc(-c3cccc4c3ccc3ccccc34)c2)c2ccc3c(ccc4ccccc43)c2)cc1 ; c1ccc(N(c2ccc(-c3cccc4c3ccc3ccccc34)cc2)c2cccc(-c3cccc4c3ccc3ccccc34)c2)cc1]

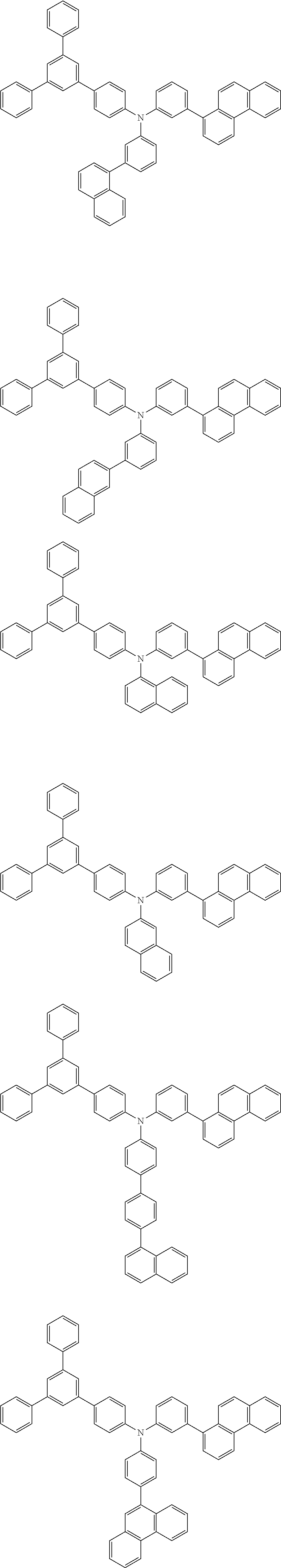

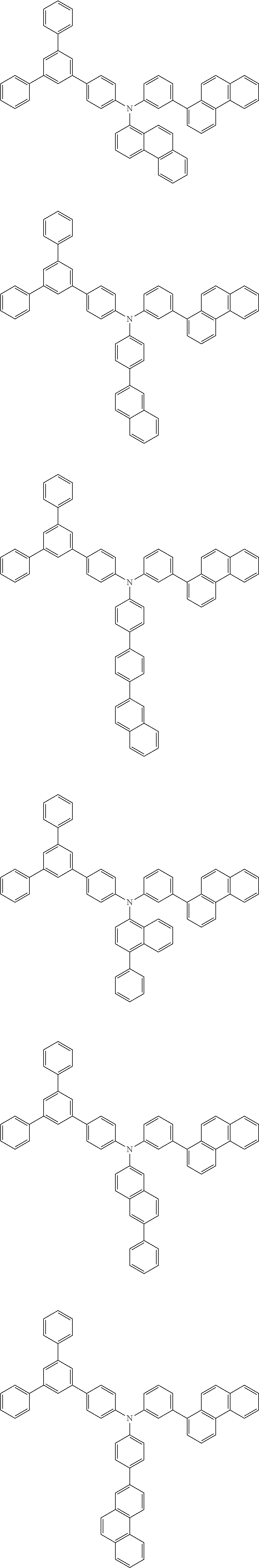

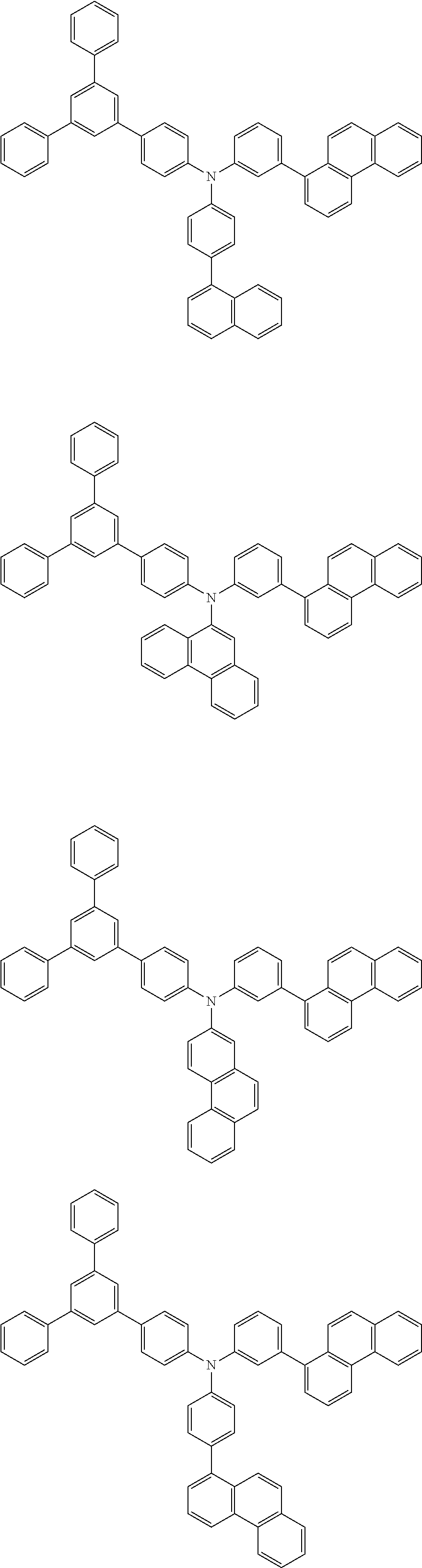 [SMILES: c1ccc(-c2cc(-c3ccccc3)cc(-c3ccc(N(c4ccc(-c5cccc6ccccc56)cc4)c4cccc(-c5cccc6c5ccc5ccccc56)c4)cc3)c2)cc1 ; c1ccc(-c2cc(-c3ccccc3)cc(-c3ccc(N(c4cccc(-c5cccc6c5ccc5ccccc56)c4)c4cc5ccccc5c5ccccc45)cc3)c2)cc1 ; c1ccc(-c2cc(-c3ccccc3)cc(-c3ccc(N(c4cccc(-c5cccc6c5ccc5ccccc56)c4)c4ccc5c(ccc6ccccc65)c4)cc3)c2)cc1 ; c1ccc(-c2cc(-c3ccccc3)cc(-c3ccc(N(c4ccc(-c5cccc6c5ccc5ccccc56)cc4)c4cccc(-c5cccc6c5ccc5ccccc56)c4)cc3)c2)cc1]

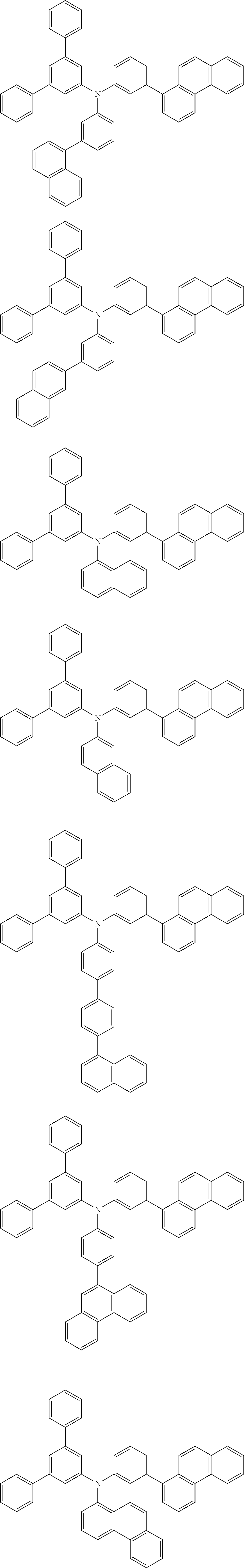

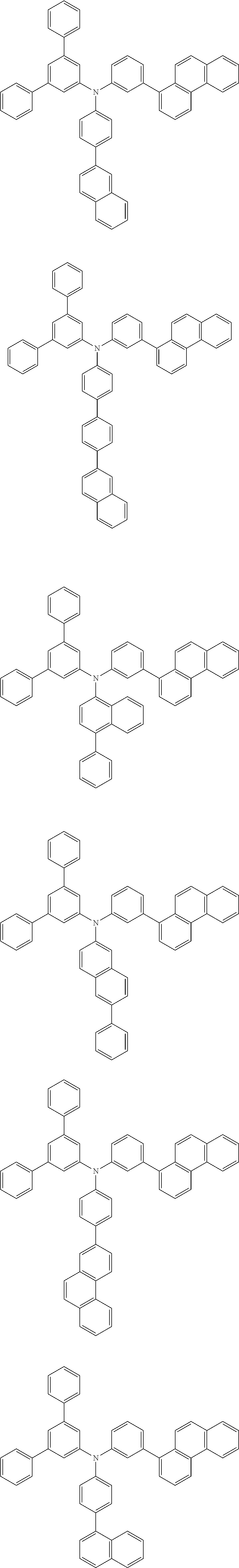

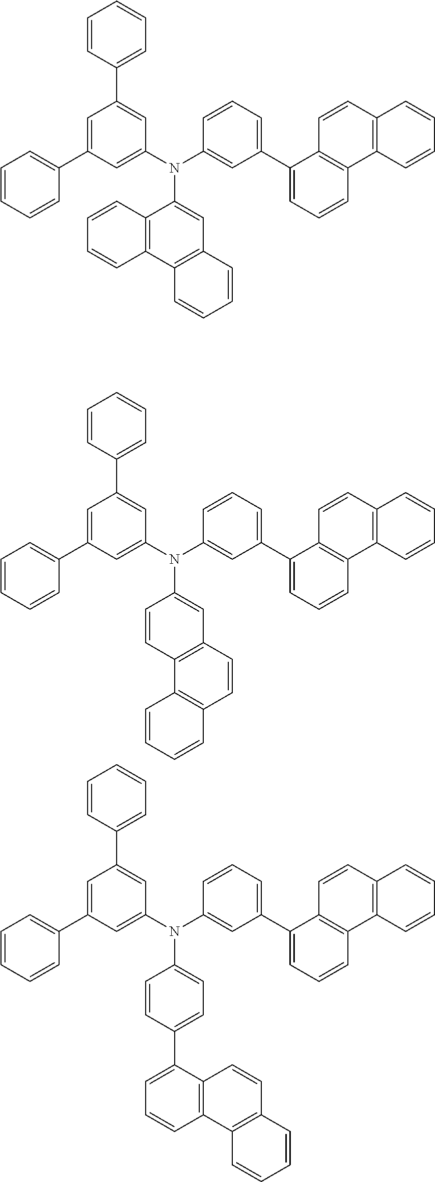

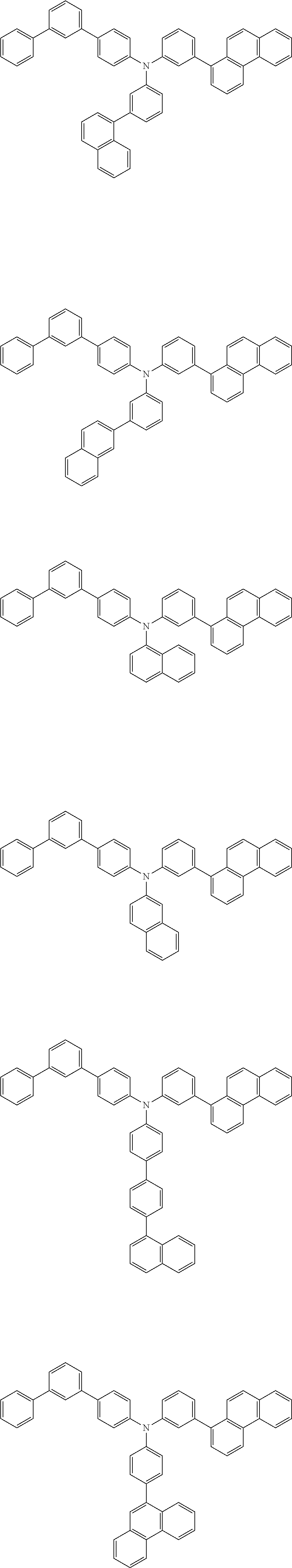

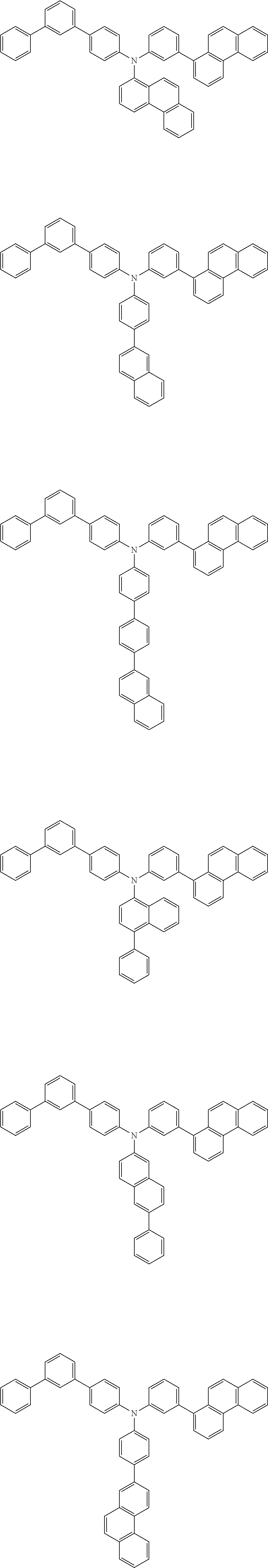

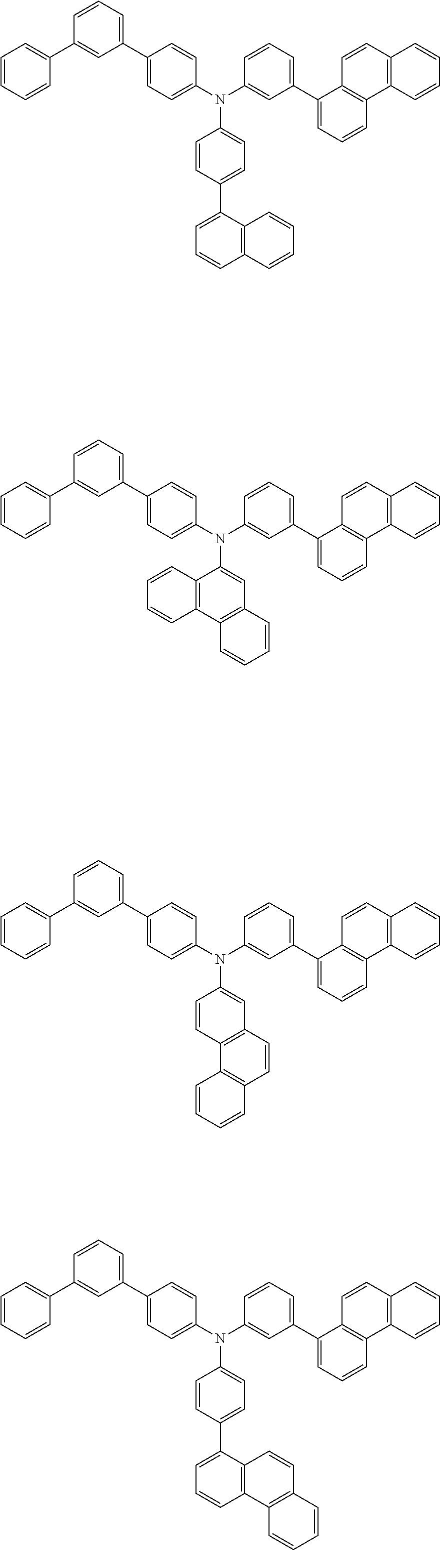 [SMILES: c1ccc(-c2cccc(-c3ccc(N(c4ccc(-c5cccc6ccccc56)cc4)c4cccc(-c5cccc6c5ccc5ccccc56)c4)cc3)c2)cc1 ; c1ccc(-c2cccc(-c3ccc(N(c4cccc(-c5cccc6c5ccc5ccccc56)c4)c4cc5ccccc5c5ccccc45)cc3)c2)cc1 ; c1ccc(-c2cccc(-c3ccc(N(c4cccc(-c5cccc6c5ccc5ccccc56)c4)c4ccc5c(ccc6ccccc65)c4)cc3)c2)cc1 ; c1ccc(-c2cccc(-c3ccc(N(c4ccc(-c5cccc6c5ccc5ccccc56)cc4)c4cccc(-c5cccc6c5ccc5ccccc56)c4)cc3)c2)cc1]

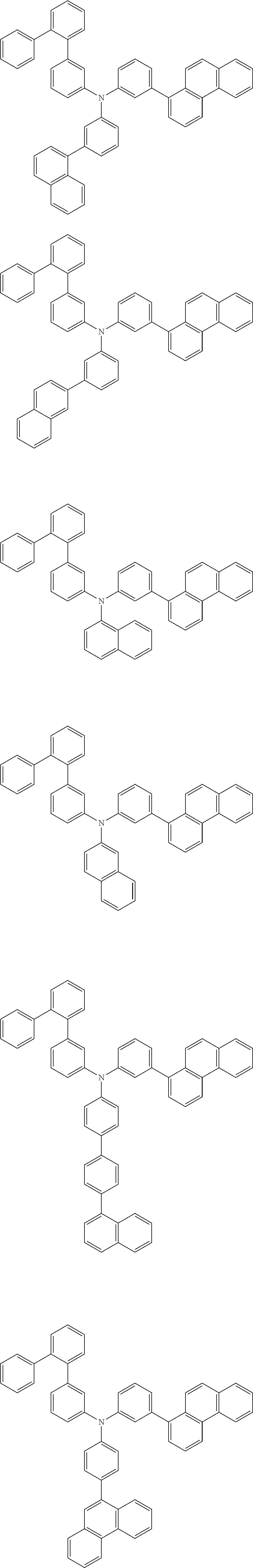

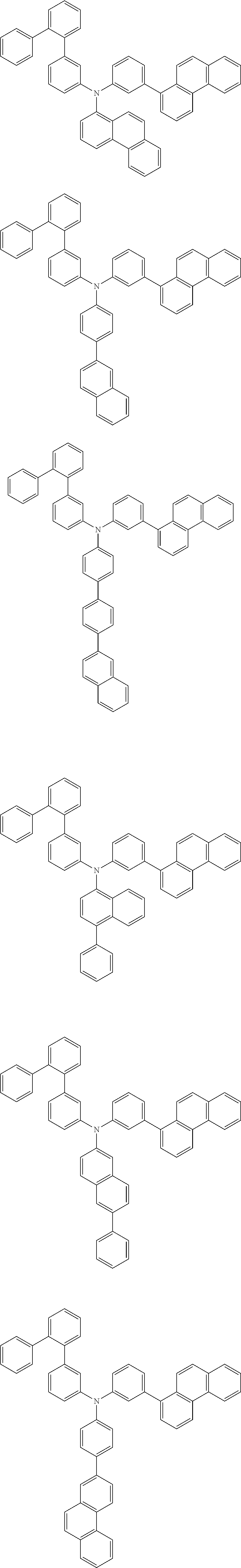

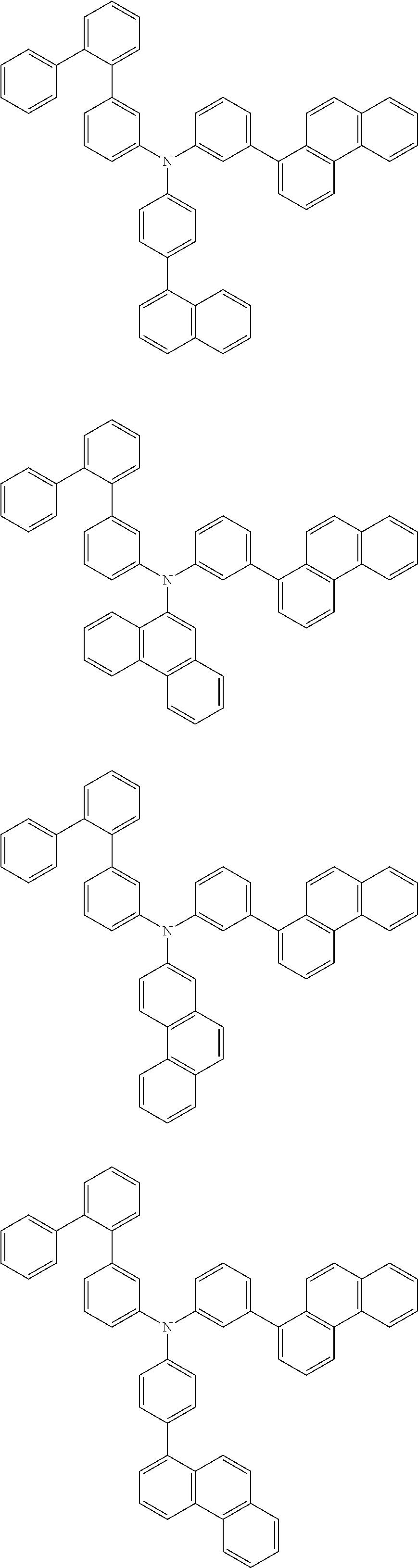 [SMILES: c1ccc(-c2ccccc2-c2cccc(N(c3ccc(-c4cccc5ccccc45)cc3)c3cccc(-c4cccc5c4ccc4ccccc45)c3)c2)cc1 ; c1ccc(-c2ccccc2-c2cccc(N(c3cccc(-c4cccc5c4ccc4ccccc45)c3)c3cc4ccccc4c4ccccc34)c2)cc1 ; c1ccc(-c2ccccc2-c2cccc(N(c3cccc(-c4cccc5c4ccc4ccccc45)c3)c3ccc4c(ccc5ccccc54)c3)c2)cc1 ; c1ccc(-c2ccccc2-c2cccc(N(c3ccc(-c4cccc5c4ccc4ccccc45)cc3)c3cccc(-c4cccc5c4ccc4ccccc45)c3)c2)cc1]

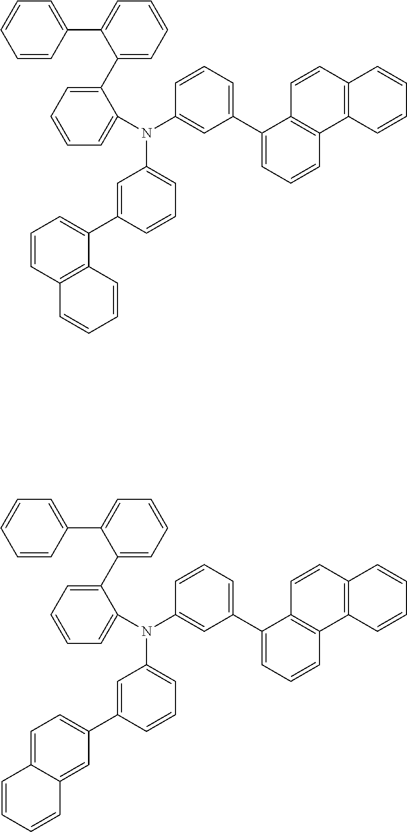 [SMILES: c1ccc(-c2ccccc2-c2ccccc2N(c2cccc(-c3cccc4ccccc34)c2)c2cccc(-c3cccc4c3ccc3ccccc34)c2)cc1 ; c1ccc(-c2ccccc2-c2ccccc2N(c2cccc(-c3ccc4ccccc4c3)c2)c2cccc(-c3cccc4c3ccc3ccccc34)c2)cc1]

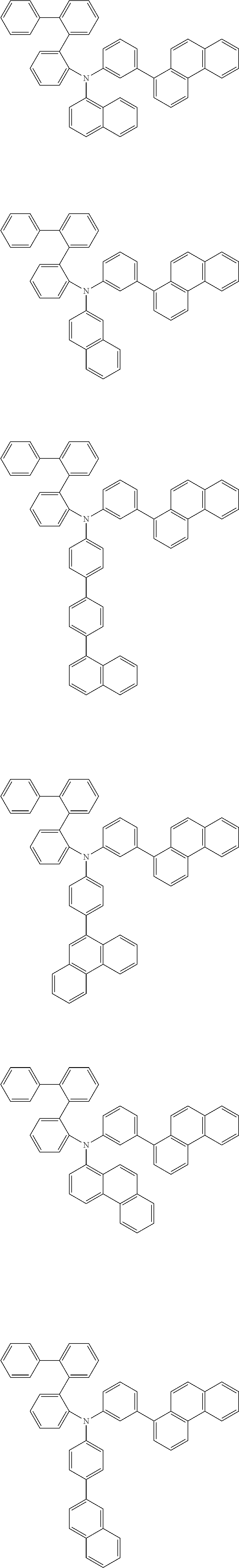

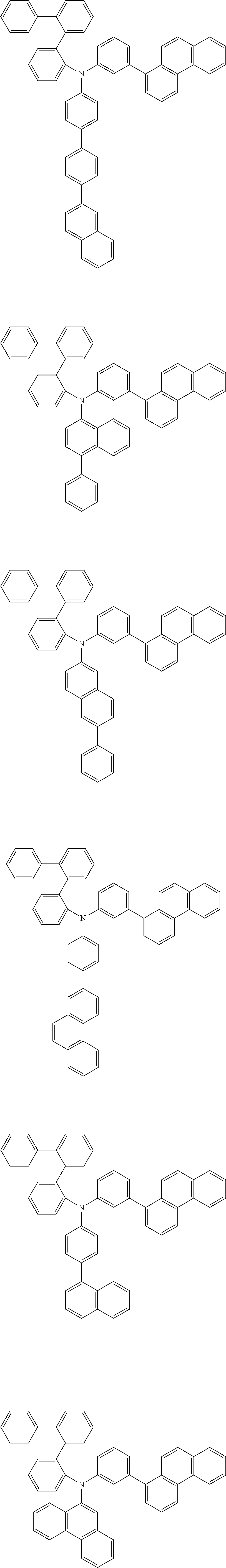

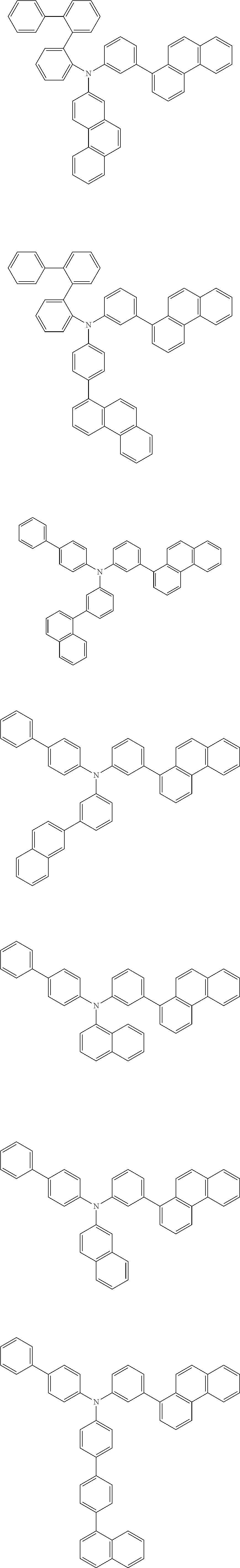

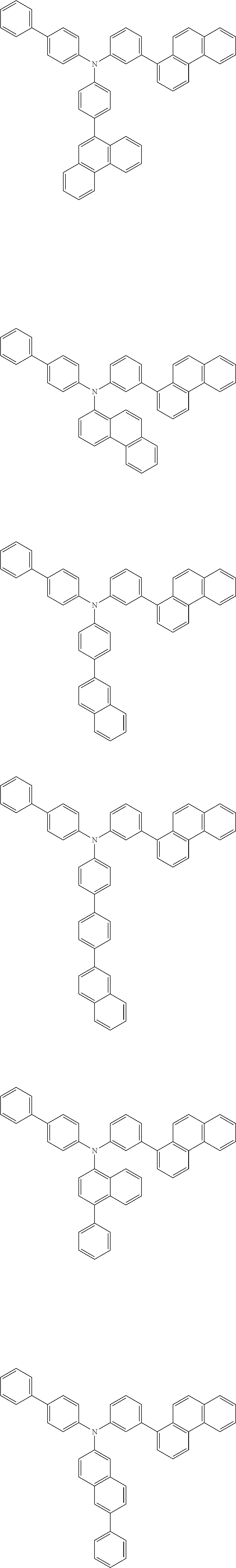

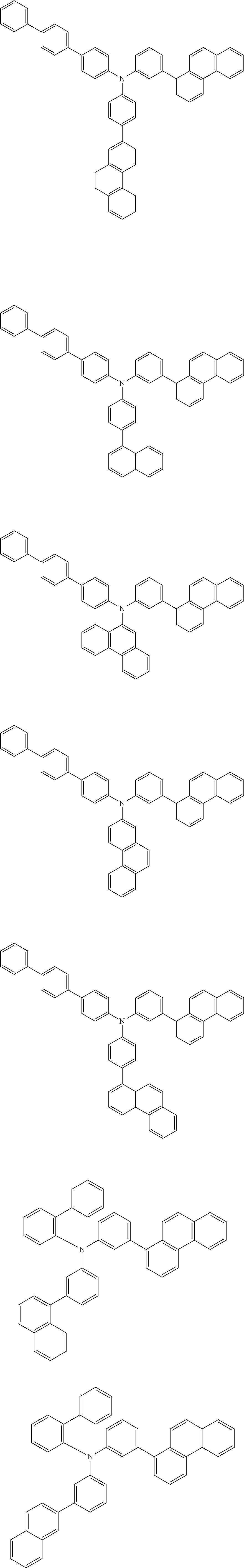

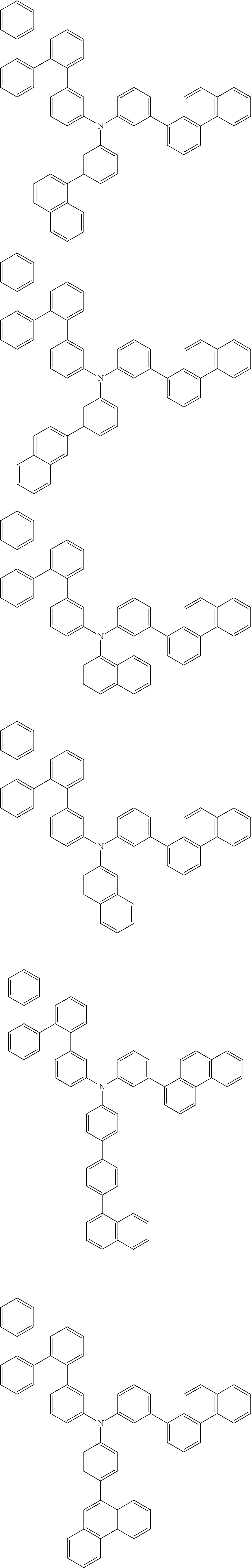

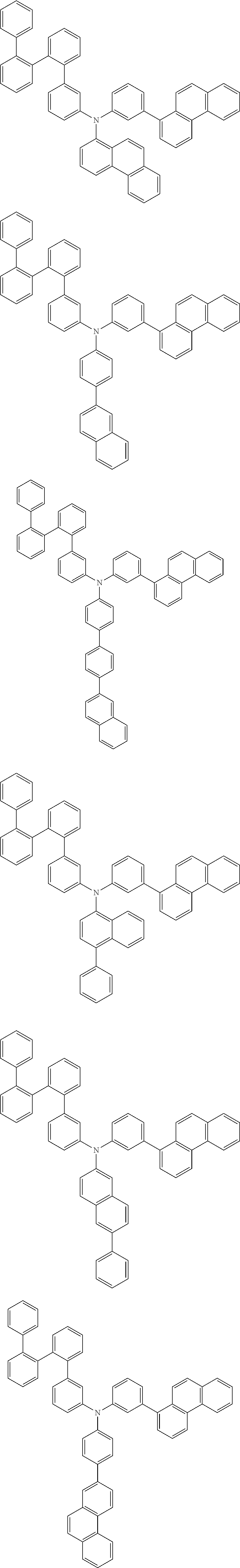

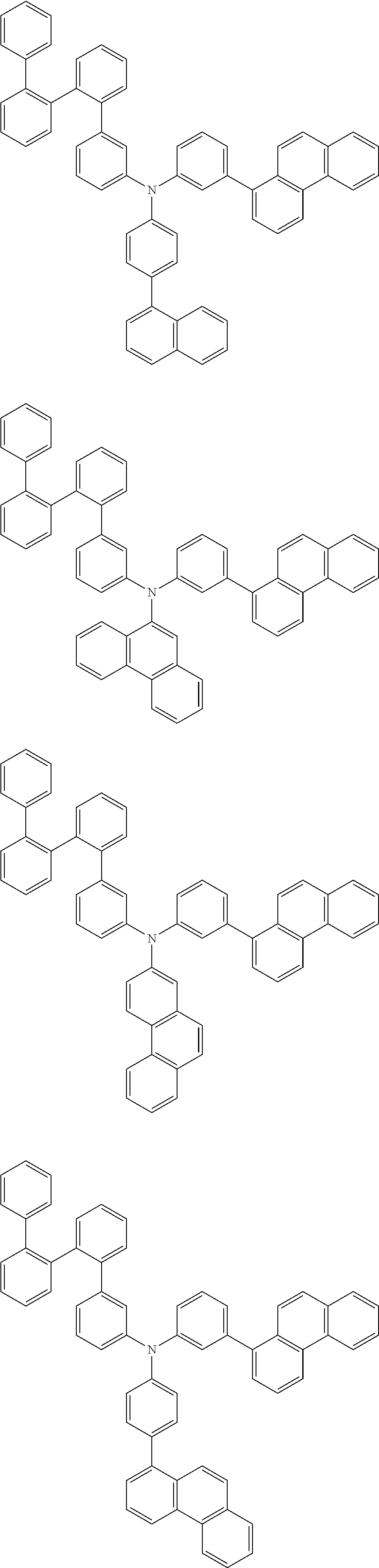 [SMILES: c1ccc(-c2ccccc2-c2ccccc2-c2cccc(N(c3ccc(-c4cccc5ccccc45)cc3)c3cccc(-c4cccc5c4ccc4ccccc45)c3)c2)cc1 ; c1ccc(-c2ccccc2-c2ccccc2-c2cccc(N(c3cccc(-c4cccc5c4ccc4ccccc45)c3)c3cc4ccccc4c4ccccc34)c2)cc1 ; c1ccc(-c2ccccc2-c2ccccc2-c2cccc(N(c3cccc(-c4cccc5c4ccc4ccccc45)c3)c3ccc4c(ccc5ccccc54)c3)c2)cc1 ; c1ccc(-c2ccccc2-c2ccccc2-c2cccc(N(c3ccc(-c4cccc5c4ccc4ccccc45)cc3)c3cccc(-c4cccc5c4ccc4ccccc45)c3)c2)cc1]

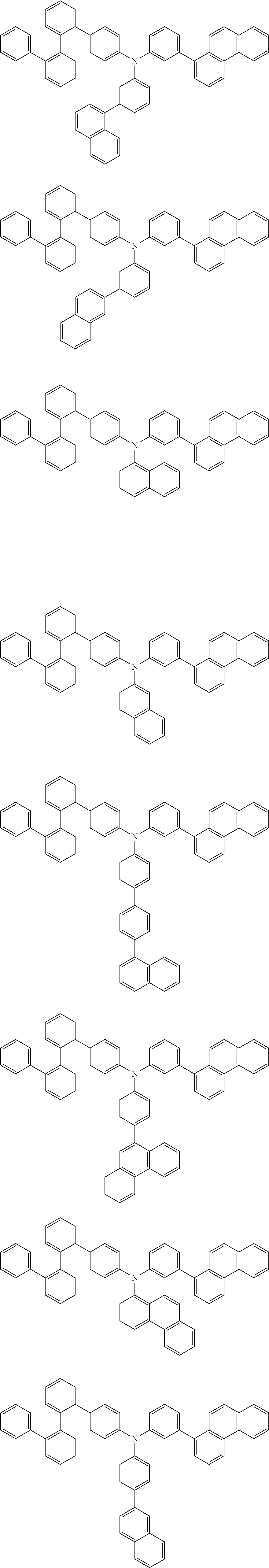

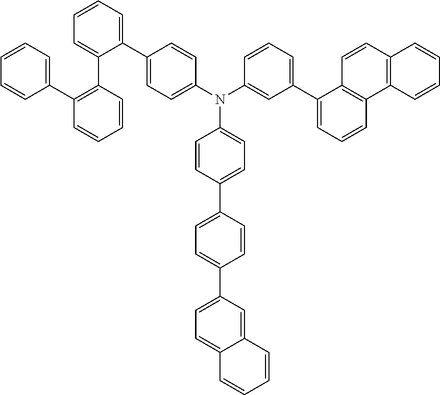 [SMILES: c1ccc(-c2ccccc2-c2ccccc2-c2ccc(N(c3ccc(-c4ccc(-c5ccc6ccccc6c5)cc4)cc3)c3cccc(-c4cccc5c4ccc4ccccc45)c3)cc2)cc1]

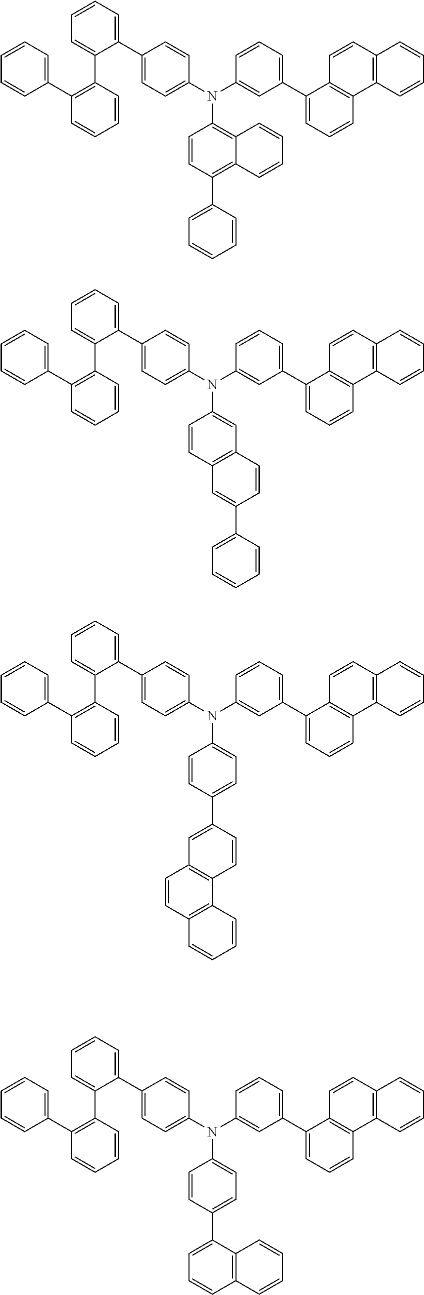 [SMILES: c1ccc(-c2ccccc2-c2ccccc2-c2ccc(N(c3cccc(-c4cccc5c4ccc4ccccc45)c3)c3ccc(-c4ccccc4)c4ccccc34)cc2)cc1 ; c1ccc(-c2ccc3cc(N(c4ccc(-c5ccccc5-c5ccccc5-c5ccccc5)cc4)c4cccc(-c5cccc6c5ccc5ccccc56)c4)ccc3c2)cc1 ; c1ccc(-c2ccccc2-c2ccccc2-c2ccc(N(c3ccc(-c4ccc5c(ccc6ccccc65)c4)cc3)c3cccc(-c4cccc5c4ccc4ccccc45)c3)cc2)cc1 ; c1ccc(-c2ccccc2-c2ccccc2-c2ccc(N(c3ccc(-c4cccc5ccccc45)cc3)c3cccc(-c4cccc5c4ccc4ccccc45)c3)cc2)cc1]

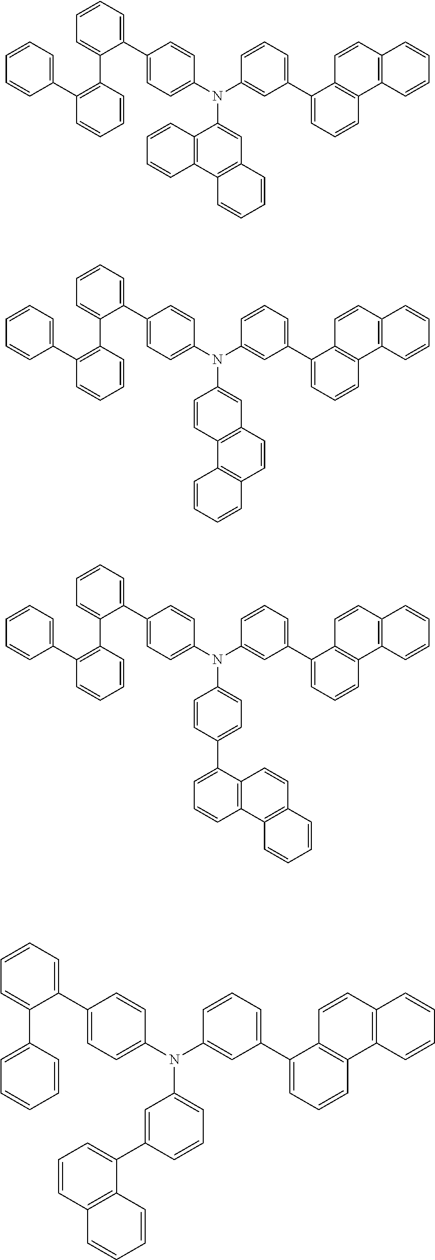 [SMILES: c1ccc(-c2ccccc2-c2ccccc2-c2ccc(N(c3cccc(-c4cccc5c4ccc4ccccc45)c3)c3cc4ccccc4c4ccccc34)cc2)cc1 ; c1ccc(-c2ccccc2-c2ccccc2-c2ccc(N(c3cccc(-c4cccc5c4ccc4ccccc45)c3)c3ccc4c(ccc5ccccc54)c3)cc2)cc1 ; c1ccc(-c2ccccc2-c2ccccc2-c2ccc(N(c3ccc(-c4cccc5c4ccc4ccccc45)cc3)c3cccc(-c4cccc5c4ccc4ccccc45)c3)cc2)cc1 ; c1ccc(-c2ccccc2-c2ccc(N(c3cccc(-c4cccc5ccccc45)c3)c3cccc(-c4cccc5c4ccc4ccccc45)c3)cc2)cc1]

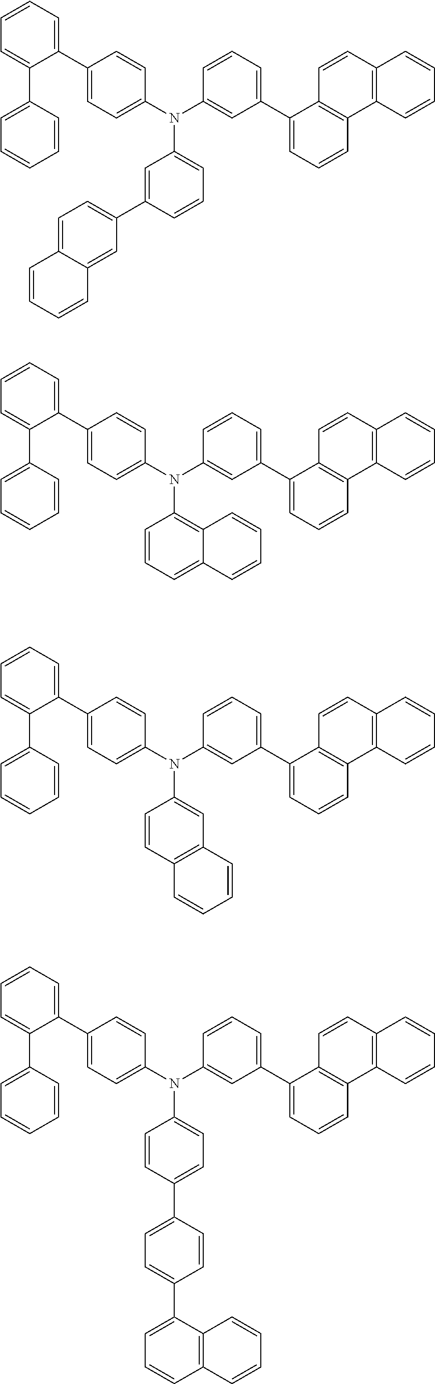 [SMILES: c1ccc(-c2ccccc2-c2ccc(N(c3cccc(-c4ccc5ccccc5c4)c3)c3cccc(-c4cccc5c4ccc4ccccc45)c3)cc2)cc1 ; c1ccc(-c2ccccc2-c2ccc(N(c3cccc(-c4cccc5c4ccc4ccccc45)c3)c3cccc4ccccc34)cc2)cc1 ; c1ccc(-c2ccccc2-c2ccc(N(c3cccc(-c4cccc5c4ccc4ccccc45)c3)c3ccc4ccccc4c3)cc2)cc1 ; c1ccc(-c2ccccc2-c2ccc(N(c3ccc(-c4ccc(-c5cccc6ccccc56)cc4)cc3)c3cccc(-c4cccc5c4ccc4ccccc45)c3)cc2)cc1]

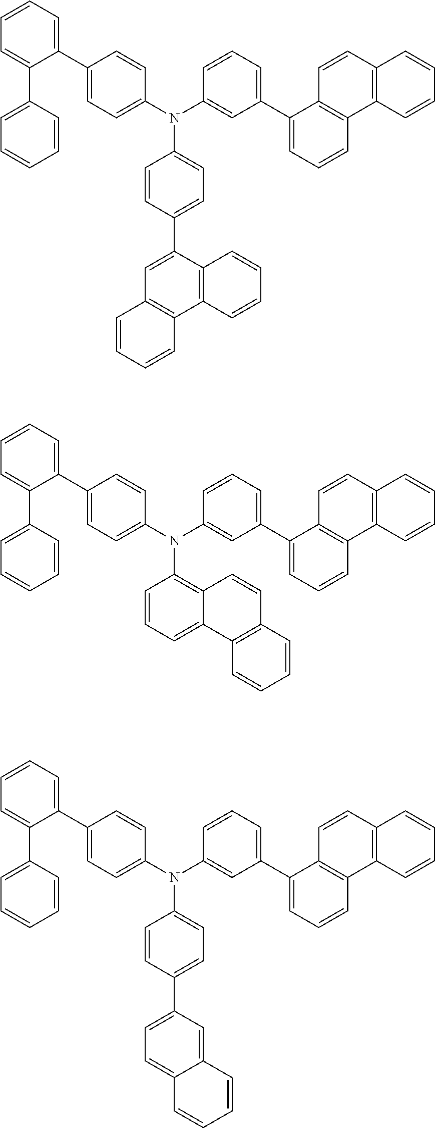 [SMILES: c1ccc(-c2ccccc2-c2ccc(N(c3ccc(-c4cc5ccccc5c5ccccc45)cc3)c3cccc(-c4cccc5c4ccc4ccccc45)c3)cc2)cc1 ; c1ccc(-c2ccccc2-c2ccc(N(c3cccc(-c4cccc5c4ccc4ccccc45)c3)c3cccc4c3ccc3ccccc34)cc2)cc1 ; c1ccc(-c2ccccc2-c2ccc(N(c3ccc(-c4ccc5ccccc5c4)cc3)c3cccc(-c4cccc5c4ccc4ccccc45)c3)cc2)cc1]

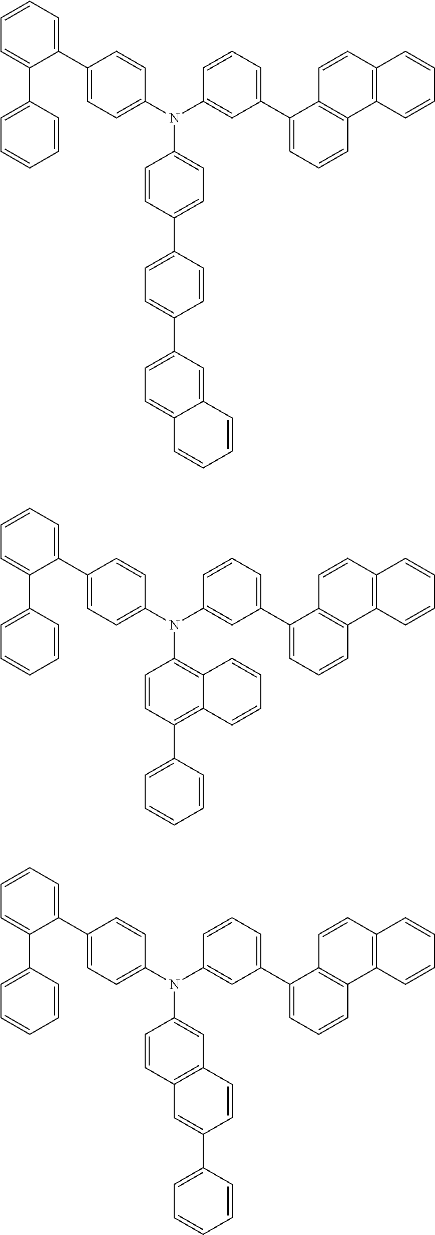 [SMILES: c1ccc(-c2ccccc2-c2ccc(N(c3ccc(-c4ccc(-c5ccc6ccccc6c5)cc4)cc3)c3cccc(-c4cccc5c4ccc4ccccc45)c3)cc2)cc1 ; c1ccc(-c2ccccc2-c2ccc(N(c3cccc(-c4cccc5c4ccc4ccccc45)c3)c3ccc(-c4ccccc4)c4ccccc34)cc2)cc1 ; c1ccc(-c2ccc3cc(N(c4ccc(-c5ccccc5-c5ccccc5)cc4)c4cccc(-c5cccc6c5ccc5ccccc56)c4)ccc3c2)cc1]

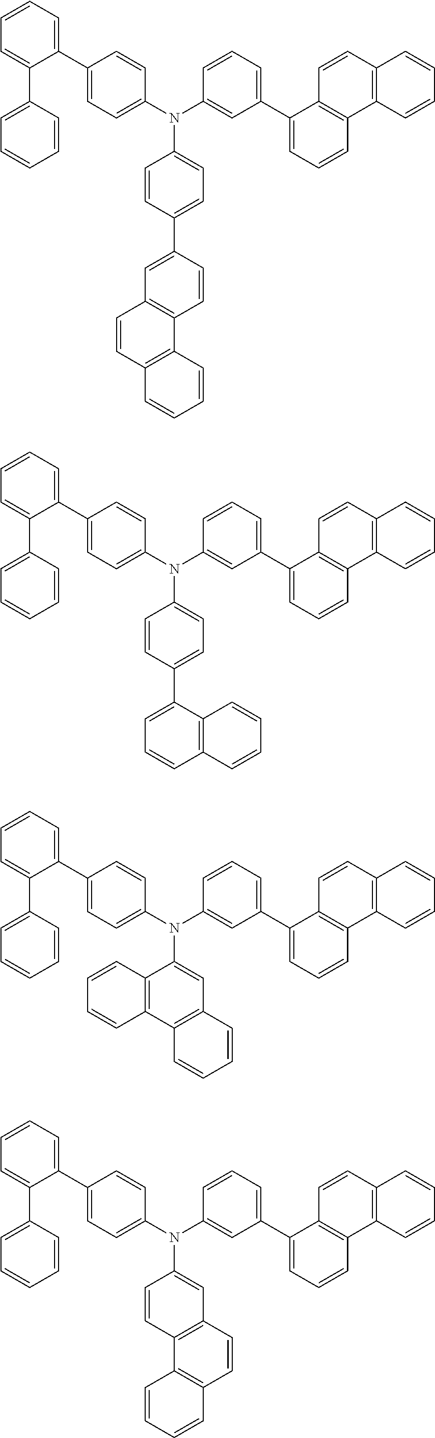 [SMILES: c1ccc(-c2ccccc2-c2ccc(N(c3ccc(-c4ccc5c(ccc6ccccc65)c4)cc3)c3cccc(-c4cccc5c4ccc4ccccc45)c3)cc2)cc1 ; c1ccc(-c2ccccc2-c2ccc(N(c3ccc(-c4cccc5ccccc45)cc3)c3cccc(-c4cccc5c4ccc4ccccc45)c3)cc2)cc1 ; c1ccc(-c2ccccc2-c2ccc(N(c3cccc(-c4cccc5c4ccc4ccccc45)c3)c3cc4ccccc4c4ccccc34)cc2)cc1 ; c1ccc(-c2ccccc2-c2ccc(N(c3cccc(-c4cccc5c4ccc4ccccc45)c3)c3ccc4c(ccc5ccccc54)c3)cc2)cc1]

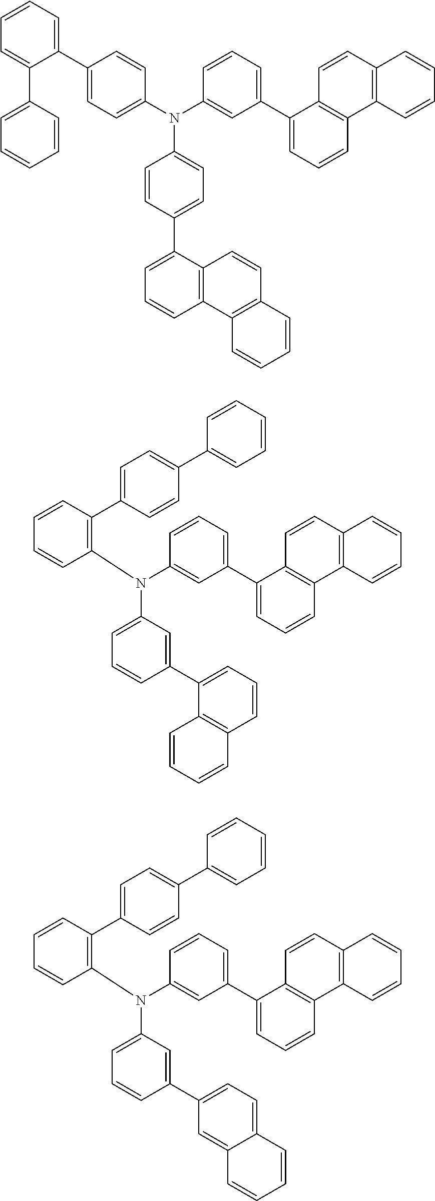

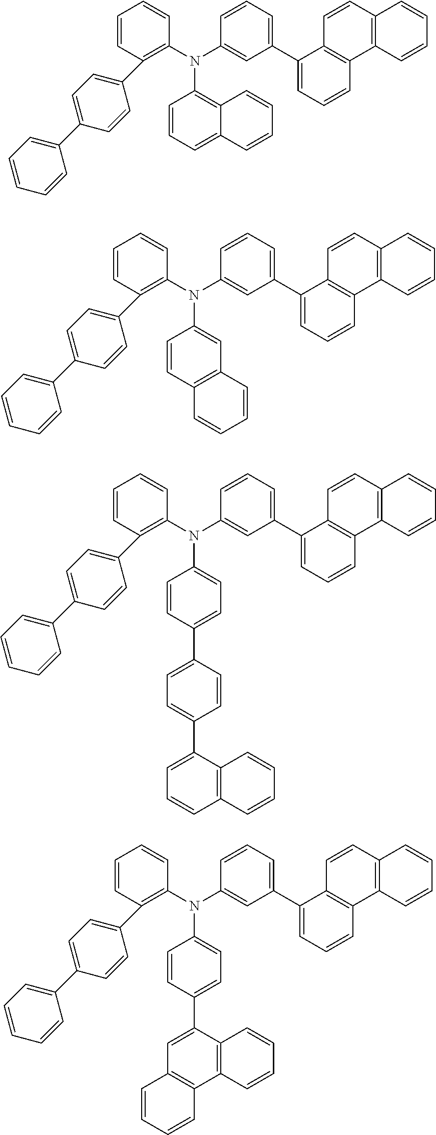

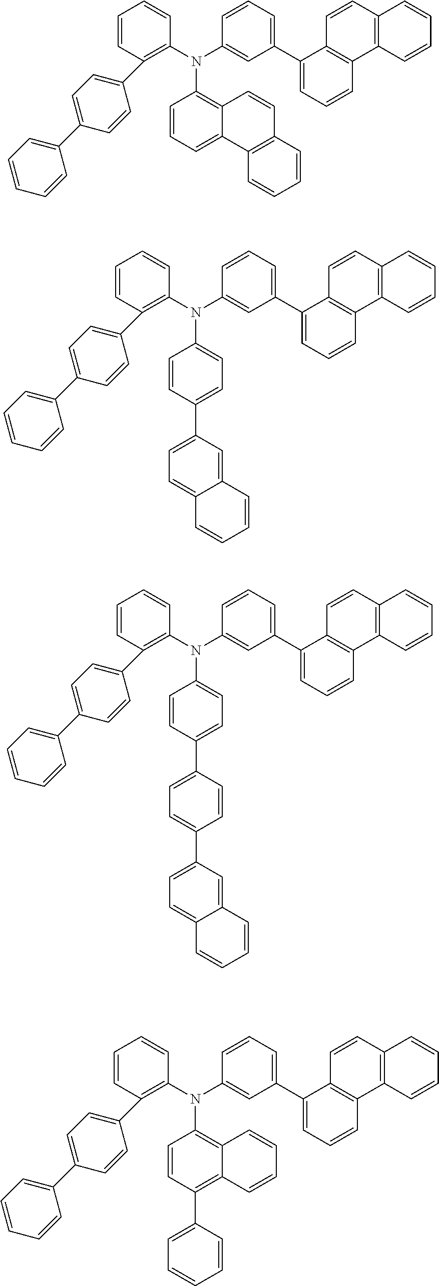 [SMILES: c1ccc(-c2ccc(-c3ccccc3N(c3cccc(-c4cccc5c4ccc4ccccc45)c3)c3cccc4c3ccc3ccccc34)cc2)cc1 ; c1ccc(-c2ccc(-c3ccccc3N(c3ccc(-c4ccc5ccccc5c4)cc3)c3cccc(-c4cccc5c4ccc4ccccc45)c3)cc2)cc1 ; c1ccc(-c2ccc(-c3ccccc3N(c3ccc(-c4ccc(-c5ccc6ccccc6c5)cc4)cc3)c3cccc(-c4cccc5c4ccc4ccccc45)c3)cc2)cc1 ; c1ccc(-c2ccc(-c3ccccc3N(c3cccc(-c4cccc5c4ccc4ccccc45)c3)c3ccc(-c4ccccc4)c4ccccc34)cc2)cc1]

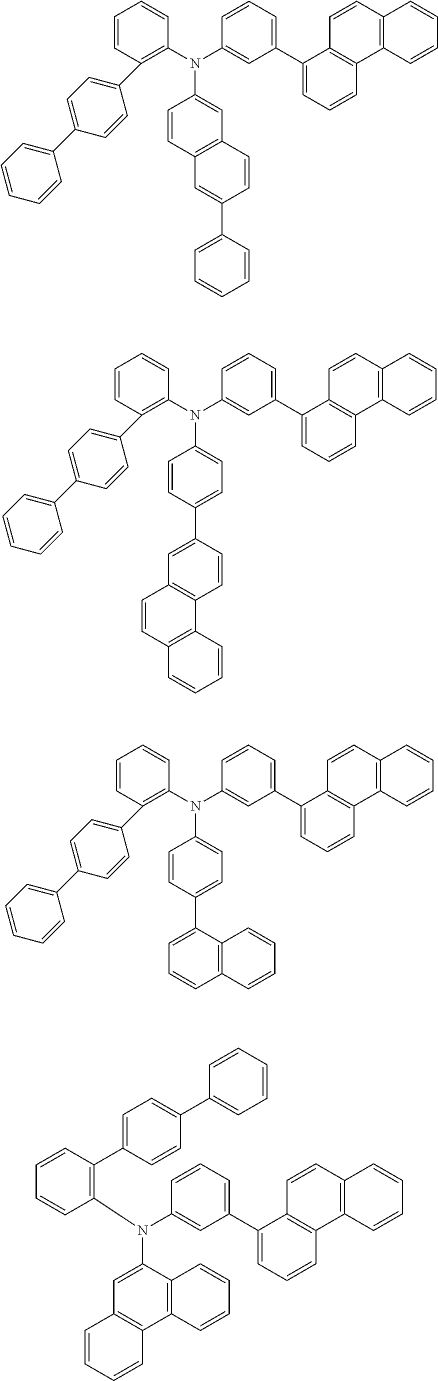 [SMILES: c1ccc(-c2ccc(-c3ccccc3N(c3cccc(-c4cccc5c4ccc4ccccc45)c3)c3ccc4cc(-c5ccccc5)ccc4c3)cc2)cc1 ; c1ccc(-c2ccc(-c3ccccc3N(c3ccc(-c4ccc5c(ccc6ccccc65)c4)cc3)c3cccc(-c4cccc5c4ccc4ccccc45)c3)cc2)cc1 ; c1ccc(-c2ccc(-c3ccccc3N(c3ccc(-c4cccc5ccccc45)cc3)c3cccc(-c4cccc5c4ccc4ccccc45)c3)cc2)cc1 ; c1ccc(-c2ccc(-c3ccccc3N(c3cccc(-c4cccc5c4ccc4ccccc45)c3)c3cc4ccccc4c4ccccc34)cc2)cc1]

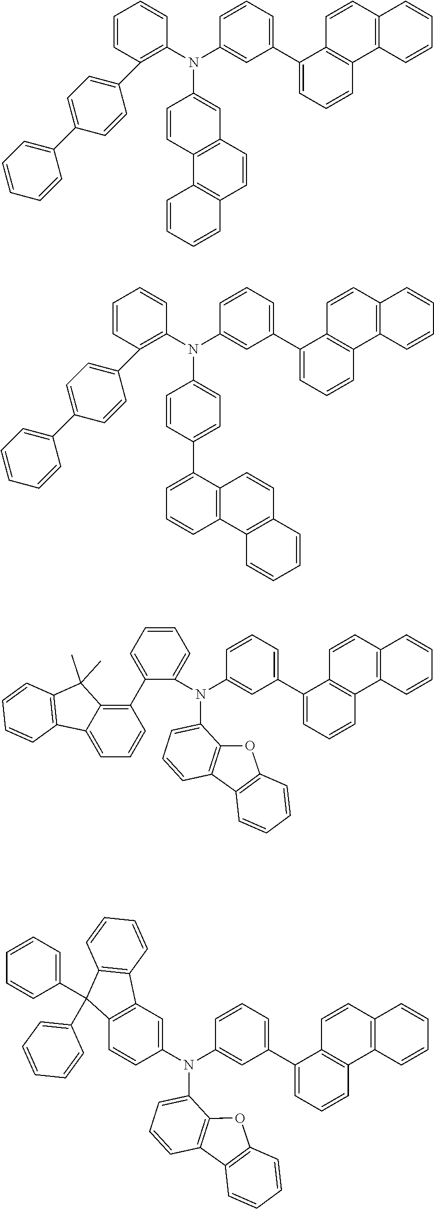 [SMILES: c1ccc(-c2ccc(-c3ccccc3N(c3cccc(-c4cccc5c4ccc4ccccc45)c3)c3ccc4c(ccc5ccccc54)c3)cc2)cc1 ; c1ccc(-c2ccc(-c3ccccc3N(c3ccc(-c4cccc5c4ccc4ccccc45)cc3)c3cccc(-c4cccc5c4ccc4ccccc45)c3)cc2)cc1 ; CC1(C)c2ccccc2-c2cccc(-c3ccccc3N(c3cccc(-c4cccc5c4ccc4ccccc45)c3)c3cccc4c3oc3ccccc34)c21 ; c1ccc(C2(c3ccccc3)c3ccccc3-c3cc(N(c4cccc(-c5cccc6c5ccc5ccccc56)c4)c4cccc5c4oc4ccccc45)ccc32)cc1]

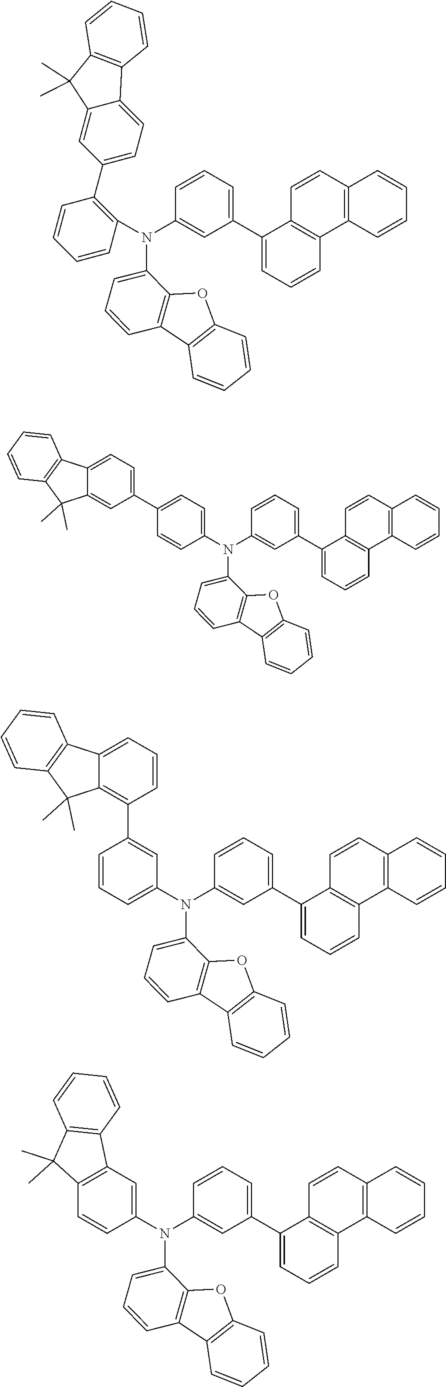 [SMILES: CC1(C)c2ccccc2-c2ccc(-c3ccccc3N(c3cccc(-c4cccc5c4ccc4ccccc45)c3)c3cccc4c3oc3ccccc34)cc21 ; CC1(C)c2ccccc2-c2ccc(-c3ccc(N(c4cccc(-c5cccc6c5ccc5ccccc56)c4)c4cccc5c4oc4ccccc45)cc3)cc21 ; CC1(C)c2ccccc2-c2cccc(-c3cccc(N(c4cccc(-c5cccc6c5ccc5ccccc56)c4)c4cccc5c4oc4ccccc45)c3)c21 ; CC1(C)c2ccccc2-c2cc(N(c3cccc(-c4cccc5c4ccc4ccccc45)c3)c3cccc4c3oc3ccccc34)ccc21]

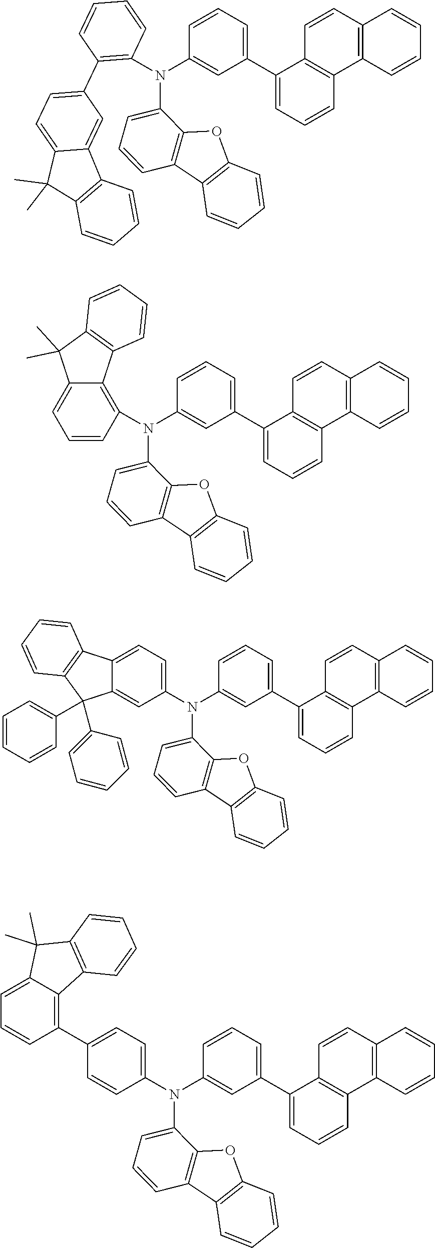

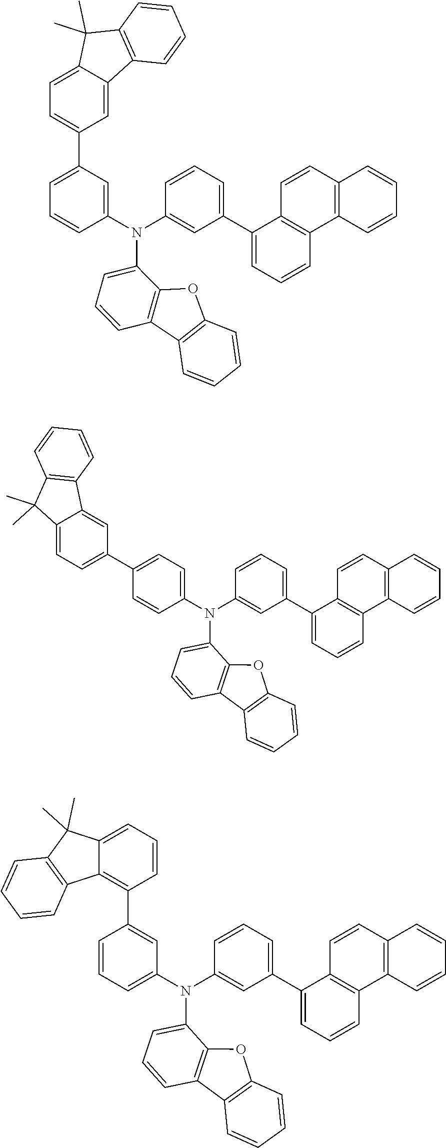 [SMILES: CC1(C)c2ccccc2-c2cc(-c3cccc(N(c4cccc(-c5cccc6c5ccc5ccccc56)c4)c4cccc5c4oc4ccccc45)c3)ccc21 ; CC1(C)c2ccccc2-c2cc(-c3ccc(N(c4cccc(-c5cccc6c5ccc5ccccc56)c4)c4cccc5c4oc4ccccc45)cc3)ccc21 ; CC1(C)c2ccccc2-c2c(-c3cccc(N(c4cccc(-c5cccc6c5ccc5ccccc56)c4)c4cccc5c4oc4ccccc45)c3)cccc21]

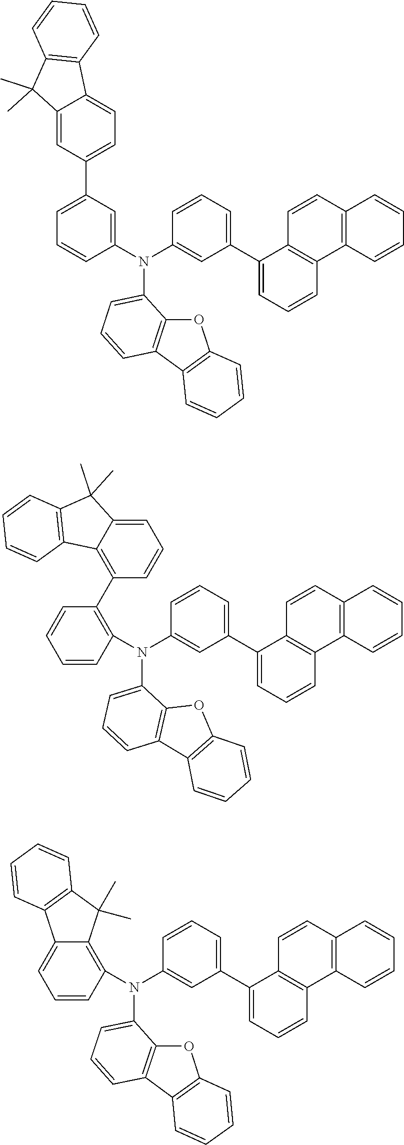 [SMILES: CC1(C)c2ccccc2-c2ccc(-c3cccc(N(c4cccc(-c5cccc6c5ccc5ccccc56)c4)c4cccc5c4oc4ccccc45)c3)cc21 ; CC1(C)c2ccccc2-c2c(-c3ccccc3N(c3cccc(-c4cccc5c4ccc4ccccc45)c3)c3cccc4c3oc3ccccc34)cccc21 ; CC1(C)c2ccccc2-c2cccc(N(c3cccc(-c4cccc5c4ccc4ccccc45)c3)c3cccc4c3oc3ccccc34)c21]

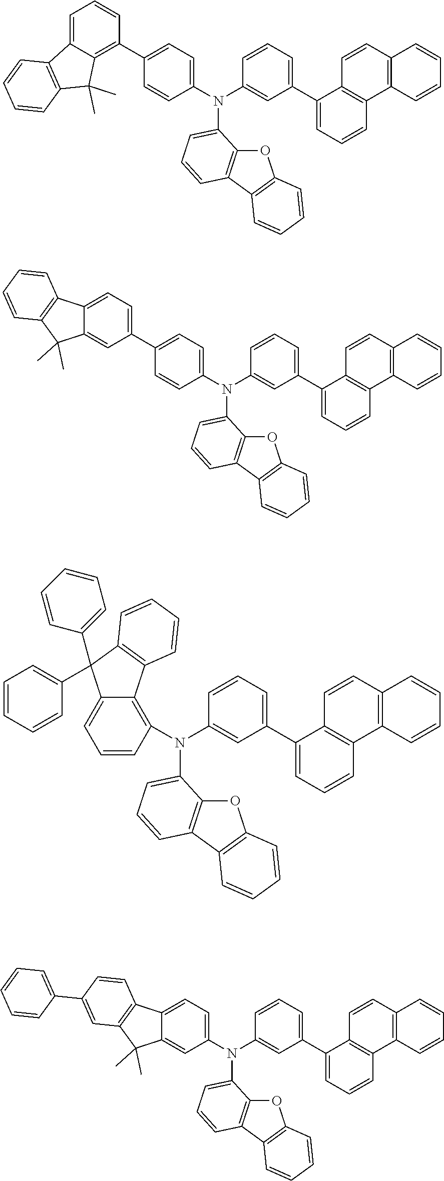 [SMILES: CC1(C)c2ccccc2-c2cccc(-c3ccc(N(c4cccc(-c5cccc6c5ccc5ccccc56)c4)c4cccc5c4oc4ccccc45)cc3)c21 ; CC1(C)c2ccccc2-c2ccc(-c3ccc(N(c4cccc(-c5cccc6c5ccc5ccccc56)c4)c4cccc5c4oc4ccccc45)cc3)cc21 ; c1ccc(C2(c3ccccc3)c3ccccc3-c3c(N(c4cccc(-c5cccc6c5ccc5ccccc56)c4)c4cccc5c4oc4ccccc45)cccc32)cc1 ; CC1(C)c2cc(-c3ccccc3)ccc2-c2ccc(N(c3cccc(-c4cccc5c4ccc4ccccc45)c3)c3cccc4c3oc3ccccc34)cc21]

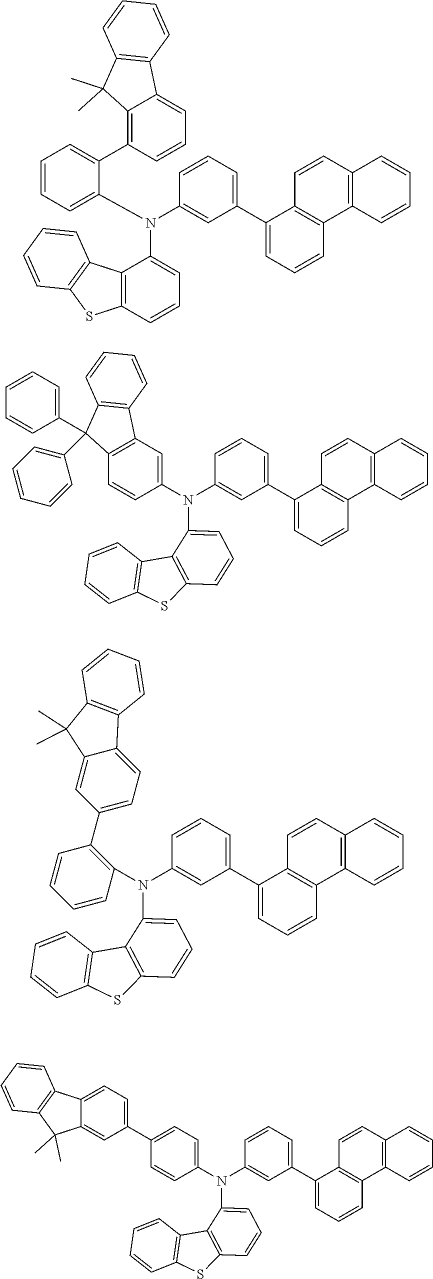

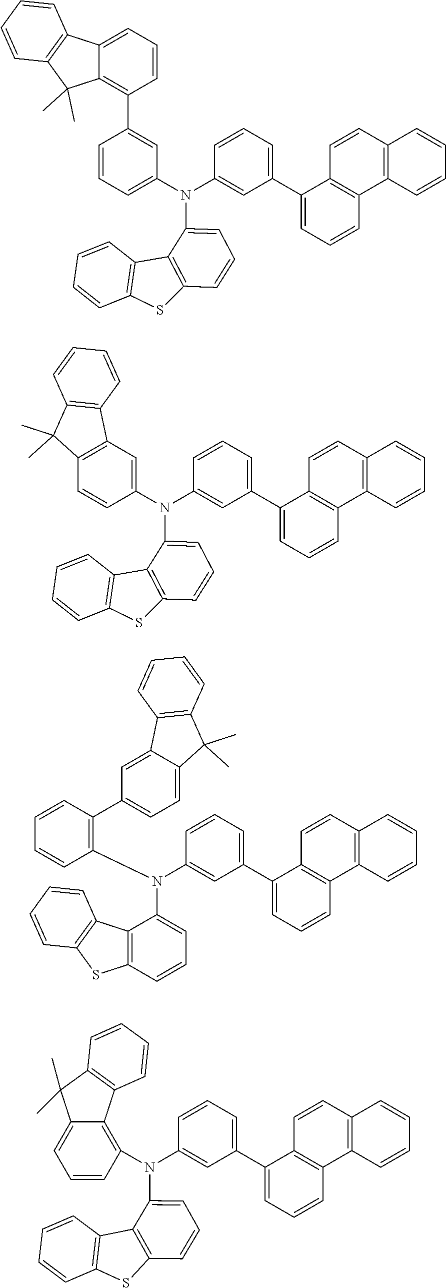

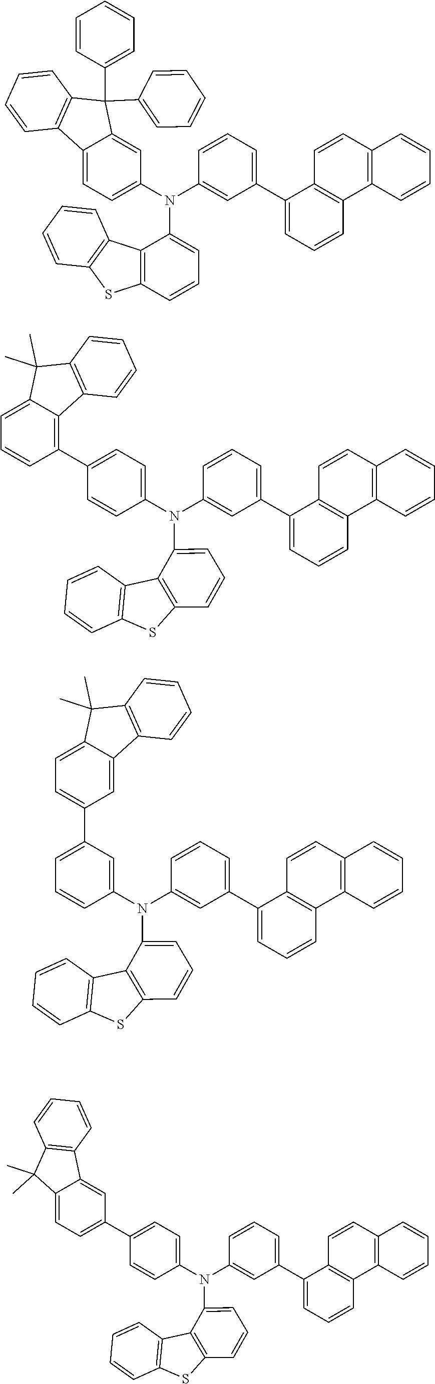

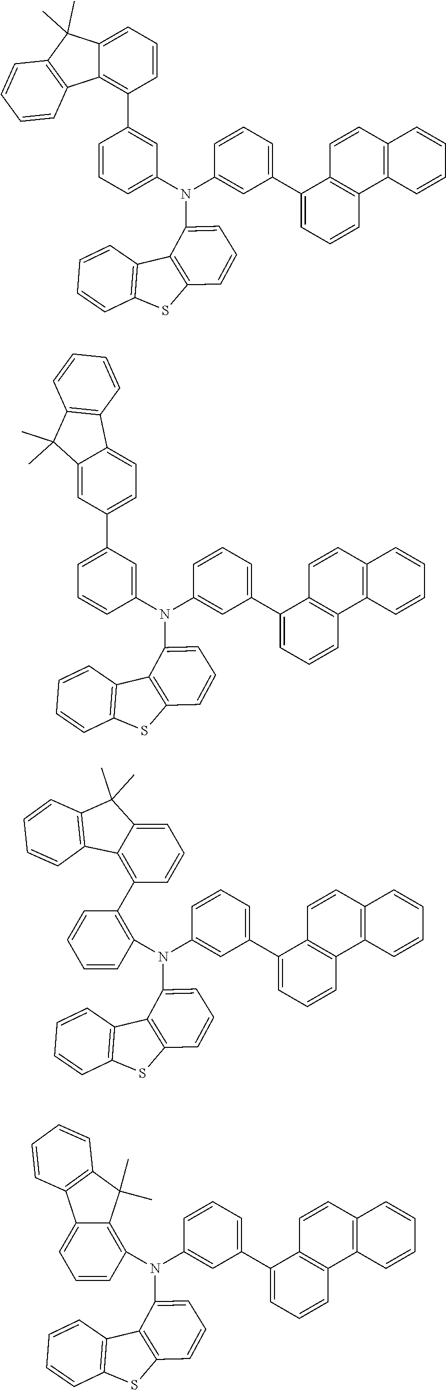

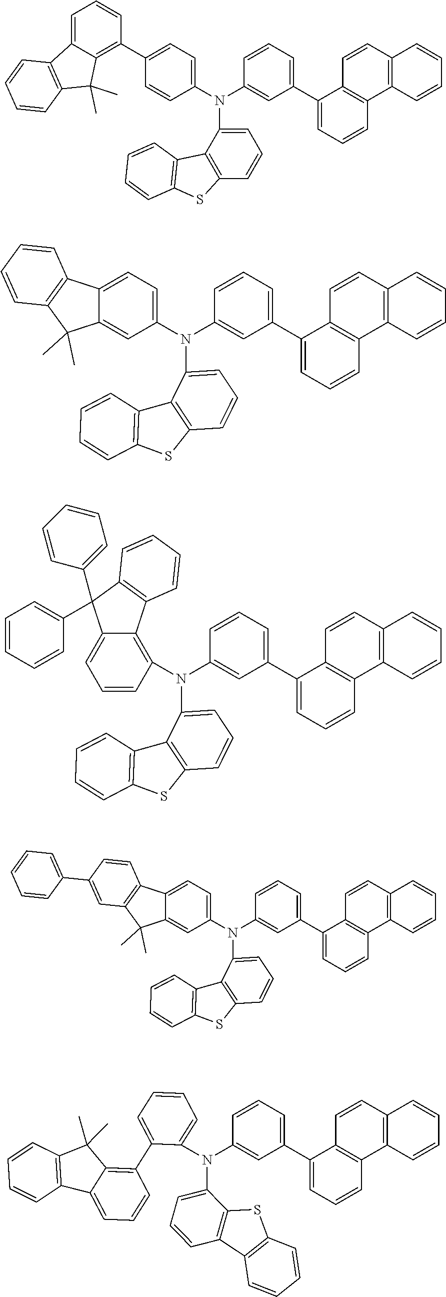 [SMILES: CC1(C)c2ccccc2-c2cccc(-c3ccc(N(c4cccc(-c5cccc6c5ccc5ccccc56)c4)c4cccc5sc6ccccc6c45)cc3)c21 ; CC1(C)c2ccccc2-c2ccc(N(c3cccc(-c4cccc5c4ccc4ccccc45)c3)c3cccc4sc5ccccc5c34)cc21 ; c1ccc(C2(c3ccccc3)c3ccccc3-c3c(N(c4cccc(-c5cccc6c5ccc5ccccc56)c4)c4cccc5sc6ccccc6c45)cccc32)cc1 ; CC1(C)c2cc(-c3ccccc3)ccc2-c2ccc(N(c3cccc(-c4cccc5c4ccc4ccccc45)c3)c3cccc4sc5ccccc5c34)cc21 ; CC1(C)c2ccccc2-c2cccc(-c3ccccc3N(c3cccc(-c4cccc5c4ccc4ccccc45)c3)c3cccc4c3sc3ccccc34)c21]

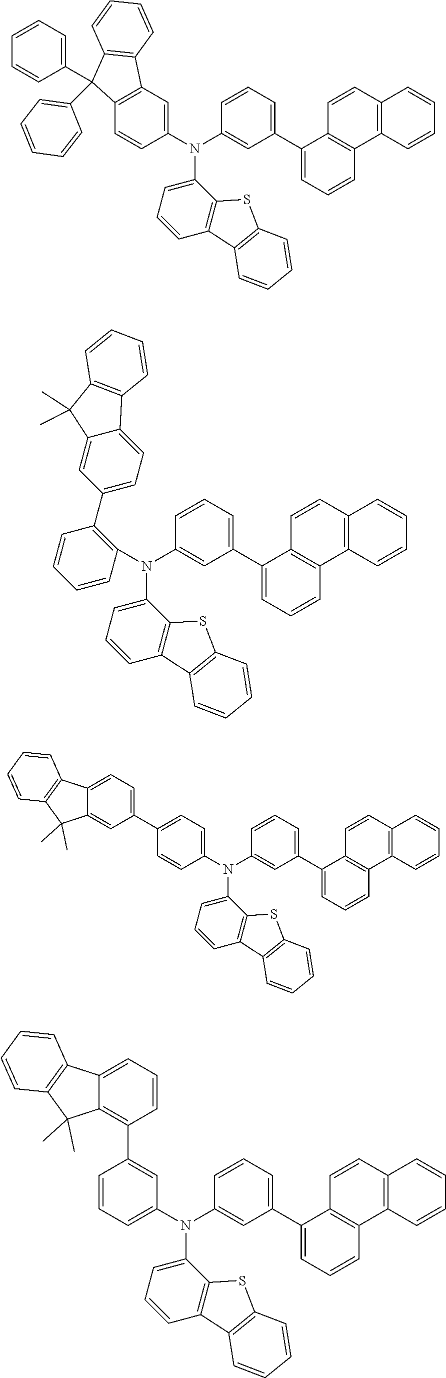 [SMILES: c1ccc(C2(c3ccccc3)c3ccccc3-c3cc(N(c4cccc(-c5cccc6c5ccc5ccccc56)c4)c4cccc5c4sc4ccccc45)ccc32)cc1 ; CC1(C)c2ccccc2-c2ccc(-c3ccccc3N(c3cccc(-c4cccc5c4ccc4ccccc45)c3)c3cccc4c3sc3ccccc34)cc21 ; CC1(C)c2ccccc2-c2ccc(-c3ccc(N(c4cccc(-c5cccc6c5ccc5ccccc56)c4)c4cccc5c4sc4ccccc45)cc3)cc21 ; CC1(C)c2ccccc2-c2cccc(-c3cccc(N(c4cccc(-c5cccc6c5ccc5ccccc56)c4)c4cccc5c4sc4ccccc45)c3)c21]

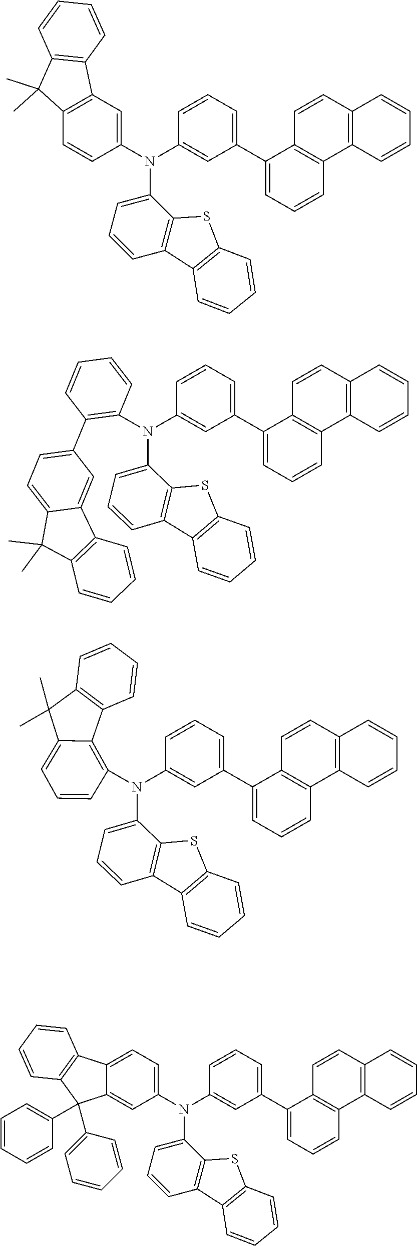

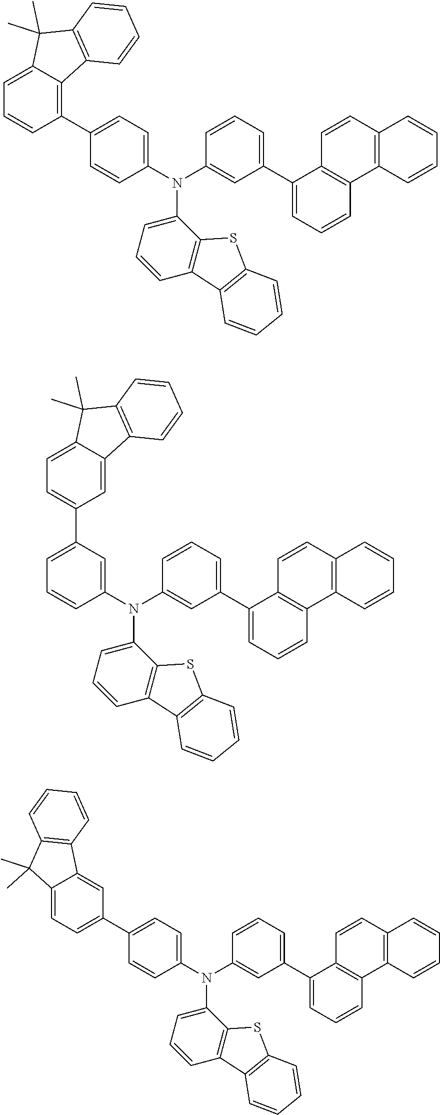 [SMILES: CC1(C)c2ccccc2-c2c(-c3ccc(N(c4cccc(-c5cccc6c5ccc5ccccc56)c4)c4cccc5c4sc4ccccc45)cc3)cccc21 ; CC1(C)c2ccccc2-c2cc(-c3cccc(N(c4cccc(-c5cccc6c5ccc5ccccc56)c4)c4cccc5c4sc4ccccc45)c3)ccc21 ; CC1(C)c2ccccc2-c2cc(-c3ccc(N(c4cccc(-c5cccc6c5ccc5ccccc56)c4)c4cccc5c4sc4ccccc45)cc3)ccc21]

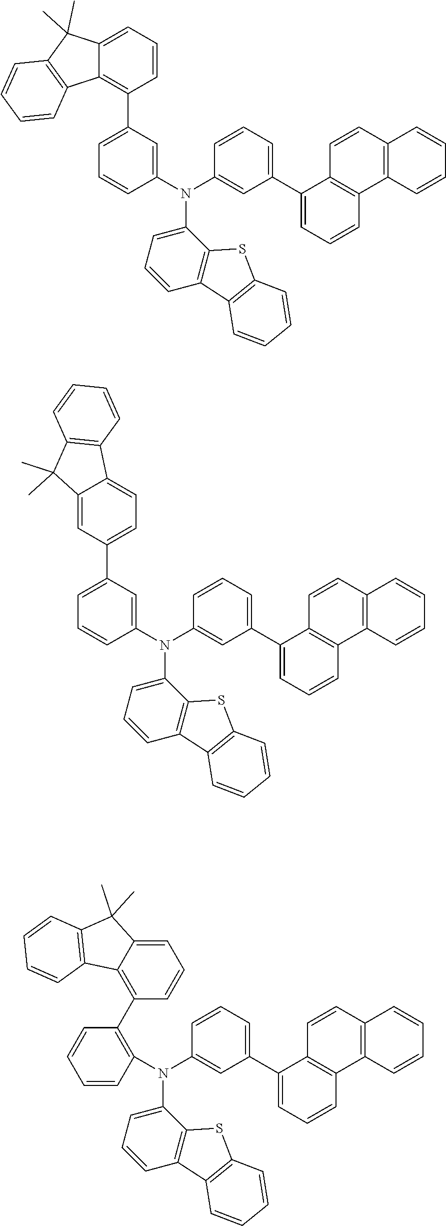 [SMILES: CC1(C)c2ccccc2-c2c(-c3cccc(N(c4cccc(-c5cccc6c5ccc5ccccc56)c4)c4cccc5c4sc4ccccc45)c3)cccc21 ; CC1(C)c2ccccc2-c2ccc(-c3cccc(N(c4cccc(-c5cccc6c5ccc5ccccc56)c4)c4cccc5c4sc4ccccc45)c3)cc21 ; CC1(C)c2ccccc2-c2c(-c3ccccc3N(c3cccc(-c4cccc5c4ccc4ccccc45)c3)c3cccc4c3sc3ccccc34)cccc21]

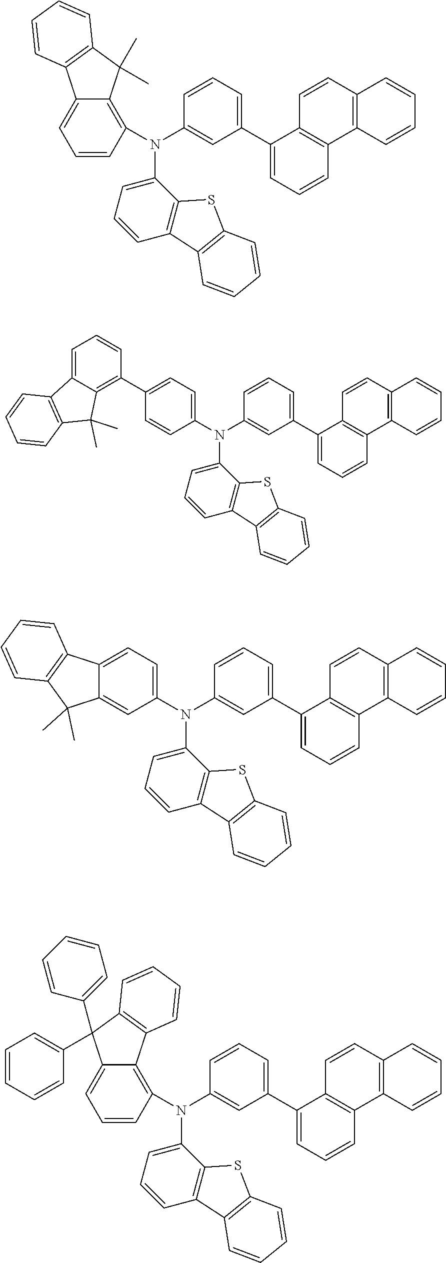 [SMILES: CC1(C)c2ccccc2-c2cccc(N(c3cccc(-c4cccc5c4ccc4ccccc45)c3)c3cccc4c3sc3ccccc34)c21 ; CC1(C)c2ccccc2-c2cccc(-c3ccc(N(c4cccc(-c5cccc6c5ccc5ccccc56)c4)c4cccc5c4sc4ccccc45)cc3)c21 ; CC1(C)c2ccccc2-c2ccc(N(c3cccc(-c4cccc5c4ccc4ccccc45)c3)c3cccc4c3sc3ccccc34)cc21 ; c1ccc(C2(c3ccccc3)c3ccccc3-c3c(N(c4cccc(-c5cccc6c5ccc5ccccc56)c4)c4cccc5c4sc4ccccc45)cccc32)cc1]

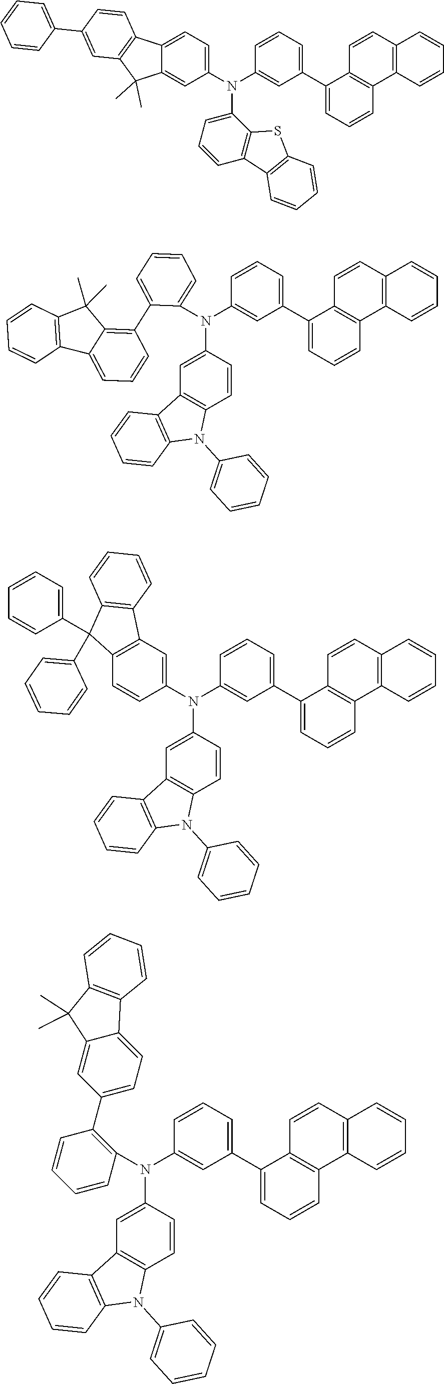 [SMILES: CC1(C)c2cc(-c3ccccc3)ccc2-c2ccc(N(c3cccc(-c4cccc5c4ccc4ccccc45)c3)c3cccc4c3sc3ccccc34)cc21 ; CC1(C)c2ccccc2-c2cccc(-c3ccccc3N(c3cccc(-c4cccc5c4ccc4ccccc45)c3)c3ccc4c(c3)c3ccccc3n4-c3ccccc3)c21 ; c1ccc(-n2c3ccccc3c3cc(N(c4cccc(-c5cccc6c5ccc5ccccc56)c4)c4ccc5c(c4)-c4ccccc4C5(c4ccccc4)c4ccccc4)ccc32)cc1 ; CC1(C)c2ccccc2-c2ccc(-c3ccccc3N(c3cccc(-c4cccc5c4ccc4ccccc45)c3)c3ccc4c(c3)c3ccccc3n4-c3ccccc3)cc21]

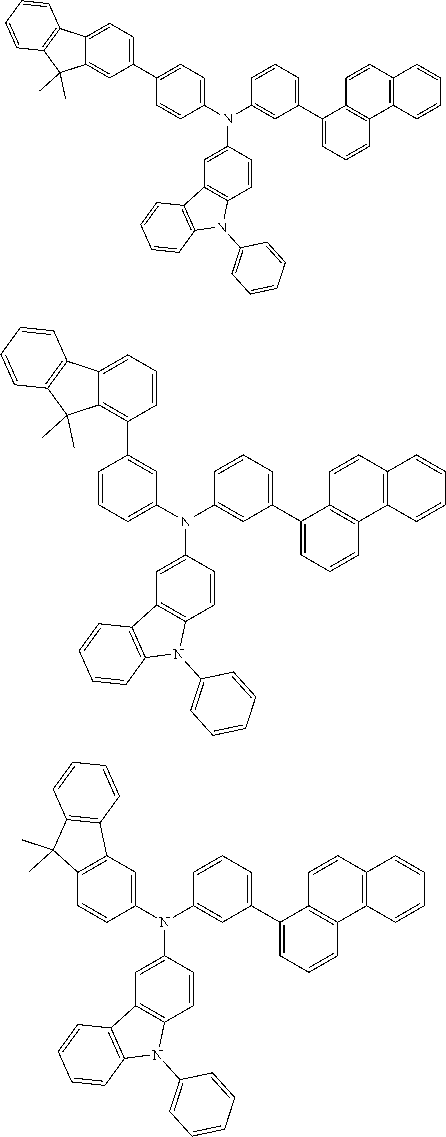 [SMILES: CC1(C)c2ccccc2-c2ccc(-c3ccc(N(c4cccc(-c5cccc6c5ccc5ccccc56)c4)c4ccc5c(c4)c4ccccc4n5-c4ccccc4)cc3)cc21 ; CC1(C)c2ccccc2-c2cccc(-c3cccc(N(c4cccc(-c5cccc6c5ccc5ccccc56)c4)c4ccc5c(c4)c4ccccc4n5-c4ccccc4)c3)c21 ; CC1(C)c2ccccc2-c2cc(N(c3cccc(-c4cccc5c4ccc4ccccc45)c3)c3ccc4c(c3)c3ccccc3n4-c3ccccc3)ccc21]

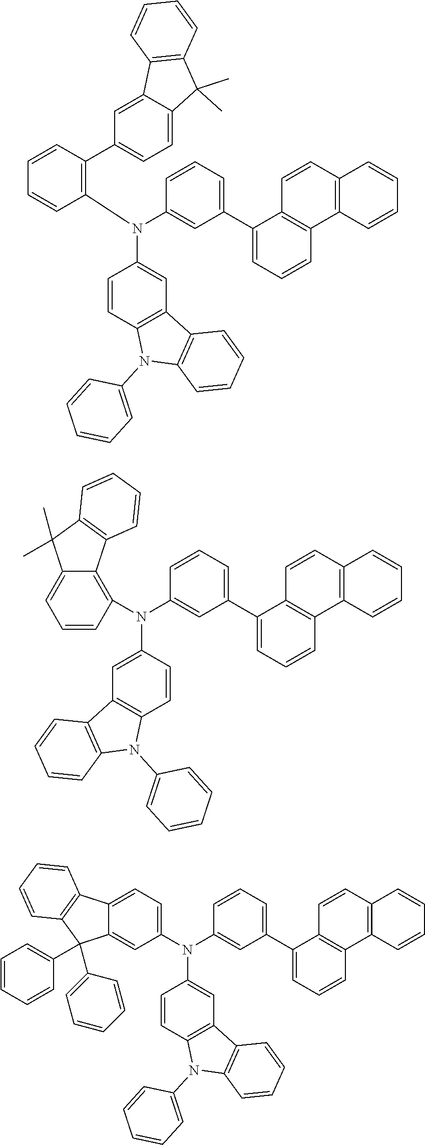

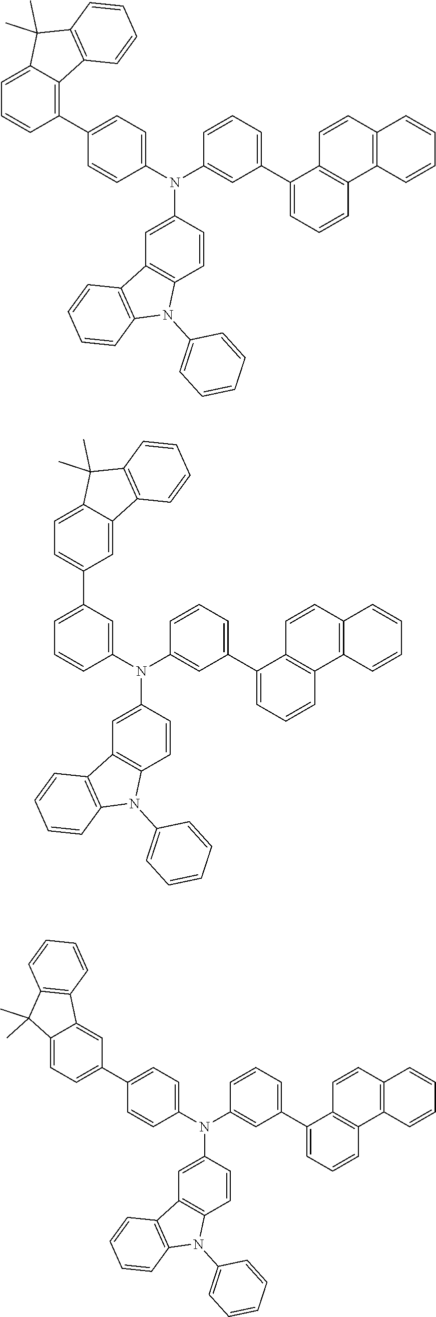 [SMILES: CC1(C)c2ccccc2-c2c(-c3ccc(N(c4cccc(-c5cccc6c5ccc5ccccc56)c4)c4ccc5c(c4)c4ccccc4n5-c4ccccc4)cc3)cccc21 ; CC1(C)c2ccccc2-c2cc(-c3cccc(N(c4cccc(-c5cccc6c5ccc5ccccc56)c4)c4ccc5c(c4)c4ccccc4n5-c4ccccc4)c3)ccc21 ; CC1(C)c2ccccc2-c2cc(-c3ccc(N(c4cccc(-c5cccc6c5ccc5ccccc56)c4)c4ccc5c(c4)c4ccccc4n5-c4ccccc4)cc3)ccc21]

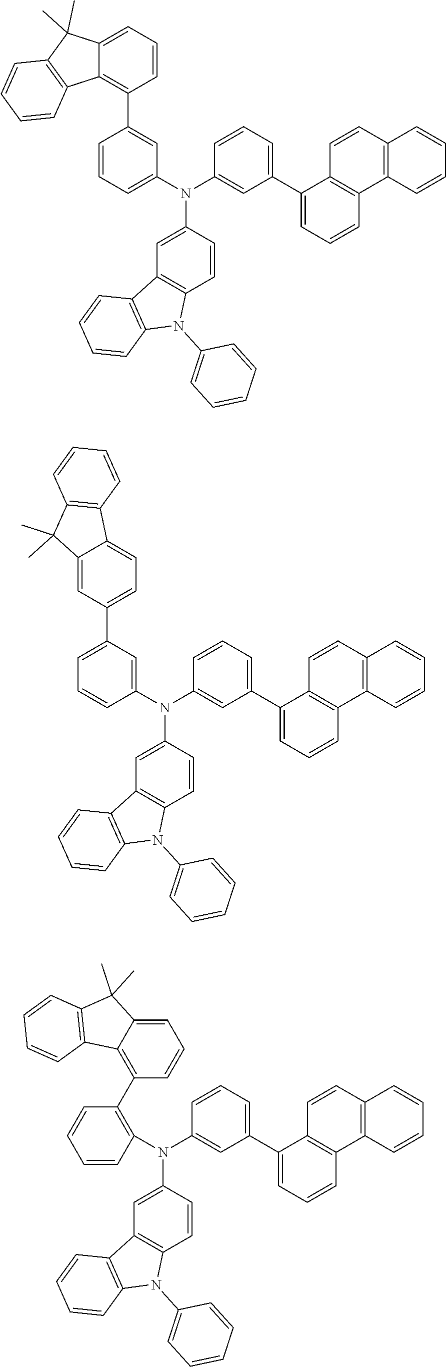 [SMILES: CC1(C)c2ccccc2-c2c(-c3cccc(N(c4cccc(-c5cccc6c5ccc5ccccc56)c4)c4ccc5c(c4)c4ccccc4n5-c4ccccc4)c3)cccc21 ; CC1(C)c2ccccc2-c2ccc(-c3cccc(N(c4cccc(-c5cccc6c5ccc5ccccc56)c4)c4ccc5c(c4)c4ccccc4n5-c4ccccc4)c3)cc21 ; CC1(C)c2ccccc2-c2c(-c3ccccc3N(c3cccc(-c4cccc5c4ccc4ccccc45)c3)c3ccc4c(c3)c3ccccc3n4-c3ccccc3)cccc21]

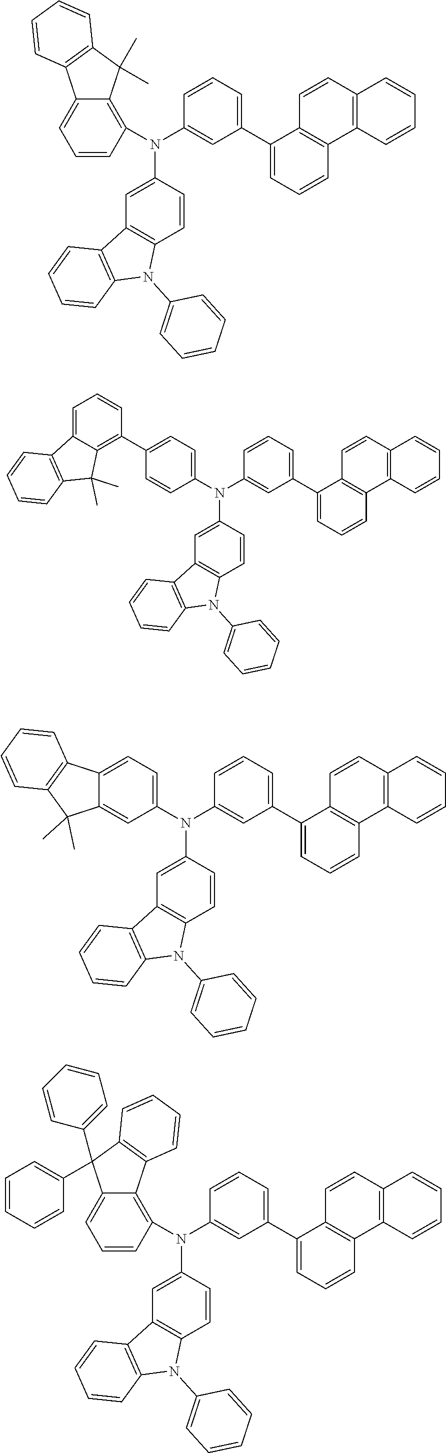 [SMILES: CC1(C)c2ccccc2-c2cccc(N(c3cccc(-c4cccc5c4ccc4ccccc45)c3)c3ccc4c(c3)c3ccccc3n4-c3ccccc3)c21 ; CC1(C)c2ccccc2-c2cccc(-c3ccc(N(c4cccc(-c5cccc6c5ccc5ccccc56)c4)c4ccc5c(c4)c4ccccc4n5-c4ccccc4)cc3)c21 ; CC1(C)c2ccccc2-c2ccc(N(c3cccc(-c4cccc5c4ccc4ccccc45)c3)c3ccc4c(c3)c3ccccc3n4-c3ccccc3)cc21 ; c1ccc(-n2c3ccccc3c3cc(N(c4cccc(-c5cccc6c5ccc5ccccc56)c4)c4cccc5c4-c4ccccc4C5(c4ccccc4)c4ccccc4)ccc32)cc1]

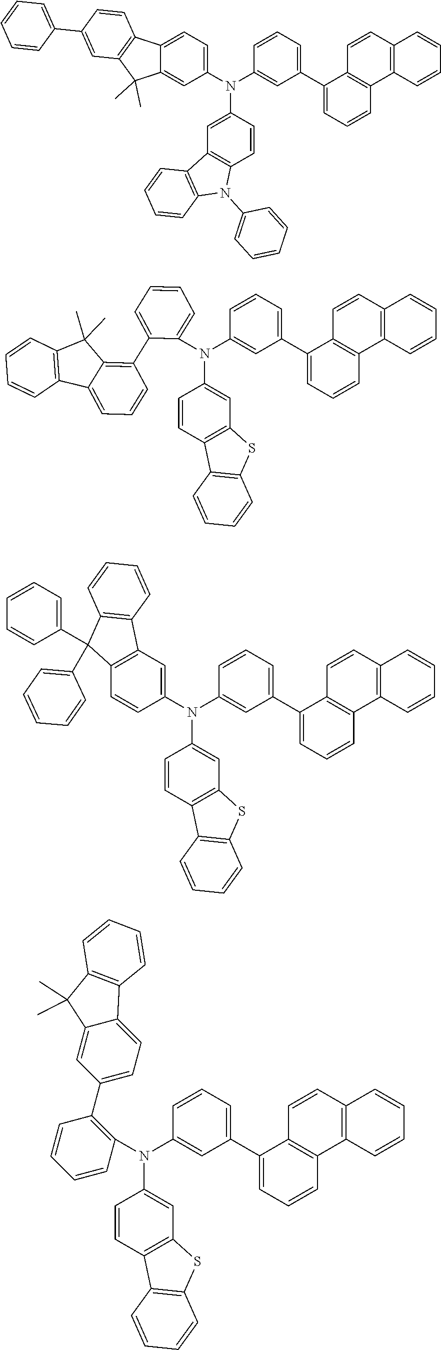

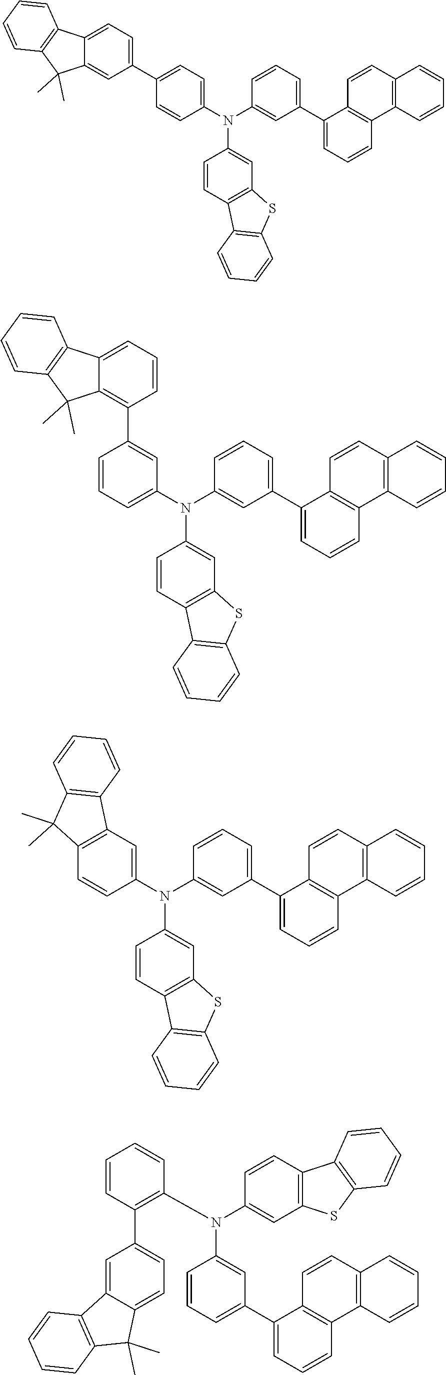 [SMILES: CC1(C)c2ccccc2-c2ccc(-c3ccc(N(c4cccc(-c5cccc6c5ccc5ccccc56)c4)c4ccc5c(c4)sc4ccccc45)cc3)cc21 ; CC1(C)c2ccccc2-c2cccc(-c3cccc(N(c4cccc(-c5cccc6c5ccc5ccccc56)c4)c4ccc5c(c4)sc4ccccc45)c3)c21 ; CC1(C)c2ccccc2-c2cc(N(c3cccc(-c4cccc5c4ccc4ccccc45)c3)c3ccc4c(c3)sc3ccccc34)ccc21 ; CC1(C)c2ccccc2-c2cc(-c3ccccc3N(c3cccc(-c4cccc5c4ccc4ccccc45)c3)c3ccc4c(c3)sc3ccccc34)ccc21]

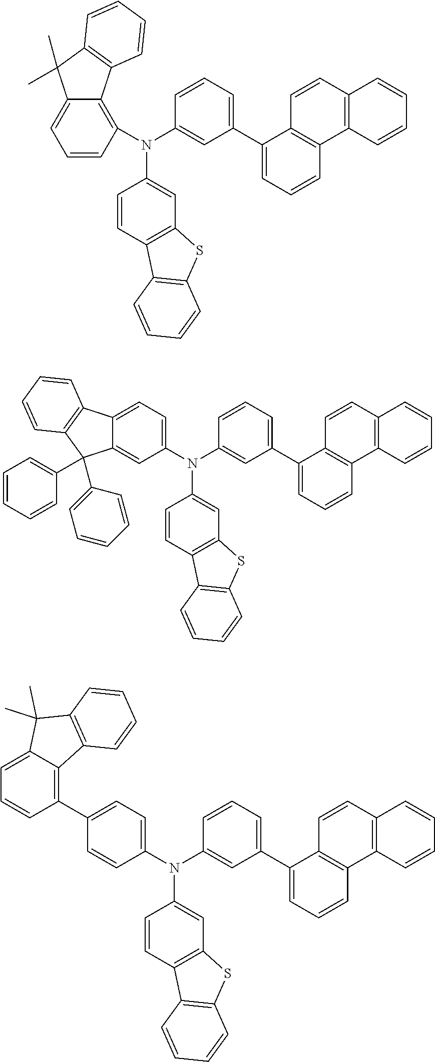 [SMILES: CC1(C)c2ccccc2-c2c(N(c3cccc(-c4cccc5c4ccc4ccccc45)c3)c3ccc4c(c3)sc3ccccc34)cccc21 ; c1ccc(C2(c3ccccc3)c3ccccc3-c3ccc(N(c4cccc(-c5cccc6c5ccc5ccccc56)c4)c4ccc5c(c4)sc4ccccc45)cc32)cc1 ; CC1(C)c2ccccc2-c2c(-c3ccc(N(c4cccc(-c5cccc6c5ccc5ccccc56)c4)c4ccc5c(c4)sc4ccccc45)cc3)cccc21]

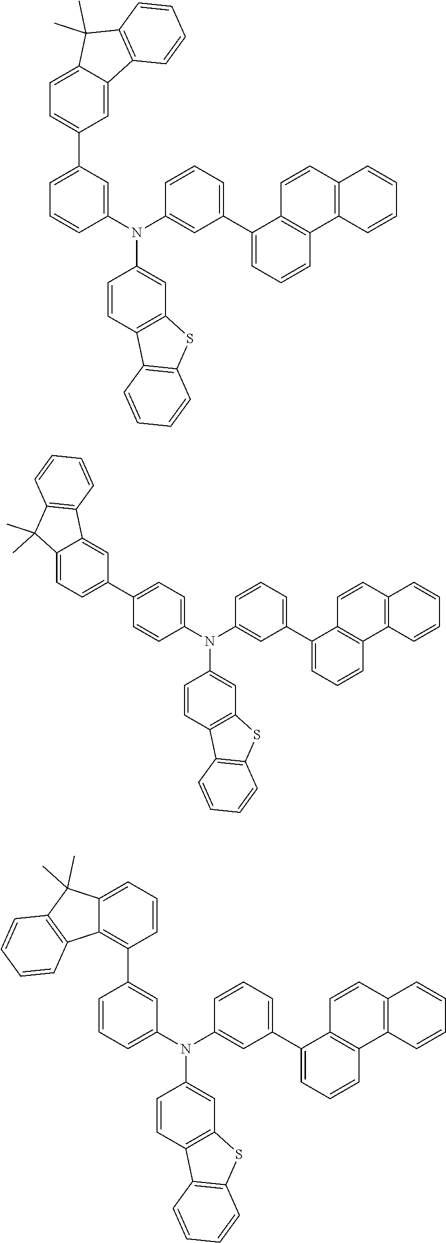 [SMILES: CC1(C)c2ccccc2-c2cc(-c3cccc(N(c4cccc(-c5cccc6c5ccc5ccccc56)c4)c4ccc5c(c4)sc4ccccc45)c3)ccc21 ; CC1(C)c2ccccc2-c2cc(-c3ccc(N(c4cccc(-c5cccc6c5ccc5ccccc56)c4)c4ccc5c(c4)sc4ccccc45)cc3)ccc21 ; CC1(C)c2ccccc2-c2c(-c3cccc(N(c4cccc(-c5cccc6c5ccc5ccccc56)c4)c4ccc5c(c4)sc4ccccc45)c3)cccc21]

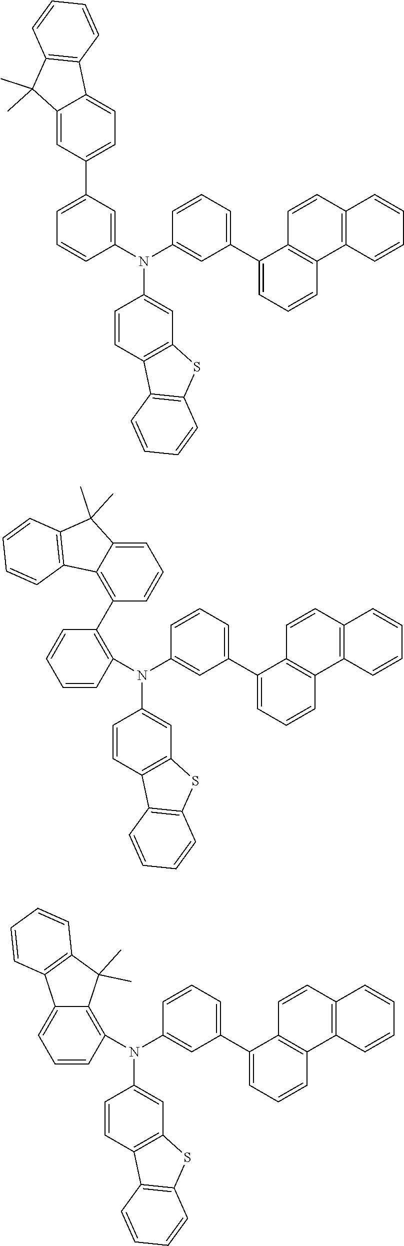 [SMILES: CC1(C)c2ccccc2-c2ccc(-c3cccc(N(c4cccc(-c5cccc6c5ccc5ccccc56)c4)c4ccc5c(c4)sc4ccccc45)c3)cc21 ; CC1(C)c2ccccc2-c2c(-c3ccccc3N(c3cccc(-c4cccc5c4ccc4ccccc45)c3)c3ccc4c(c3)sc3ccccc34)cccc21 ; CC1(C)c2ccccc2-c2cccc(N(c3cccc(-c4cccc5c4ccc4ccccc45)c3)c3ccc4c(c3)sc3ccccc34)c21]

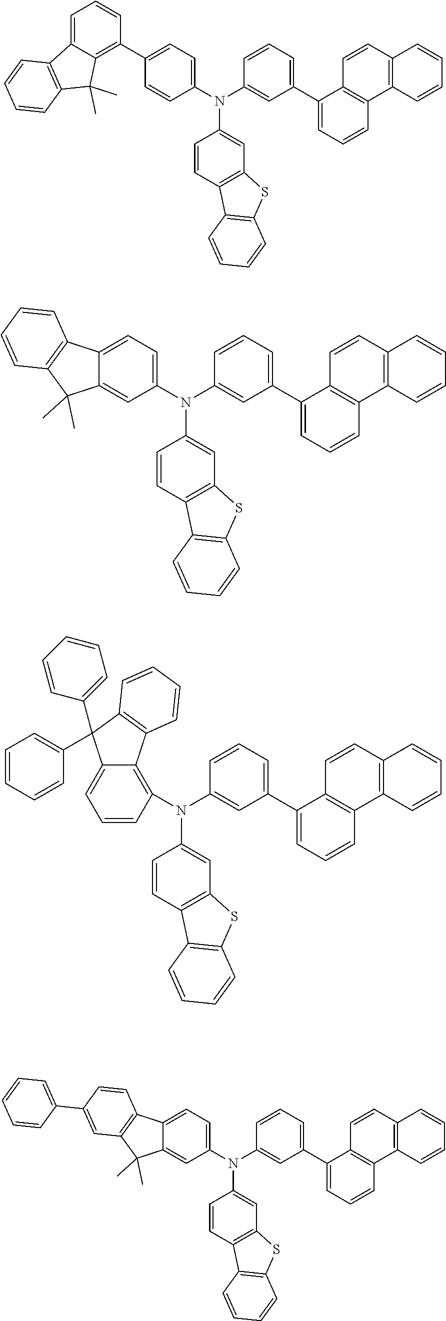 [SMILES: CC1(C)c2ccccc2-c2cccc(-c3ccc(N(c4cccc(-c5cccc6c5ccc5ccccc56)c4)c4ccc5c(c4)sc4ccccc45)cc3)c21 ; CC1(C)c2ccccc2-c2ccc(N(c3cccc(-c4cccc5c4ccc4ccccc45)c3)c3ccc4c(c3)sc3ccccc34)cc21 ; c1ccc(C2(c3ccccc3)c3ccccc3-c3c(N(c4cccc(-c5cccc6c5ccc5ccccc56)c4)c4ccc5c(c4)sc4ccccc45)cccc32)cc1 ; CC1(C)c2cc(-c3ccccc3)ccc2-c2ccc(N(c3cccc(-c4cccc5c4ccc4ccccc45)c3)c3ccc4c(c3)sc3ccccc34)cc21]

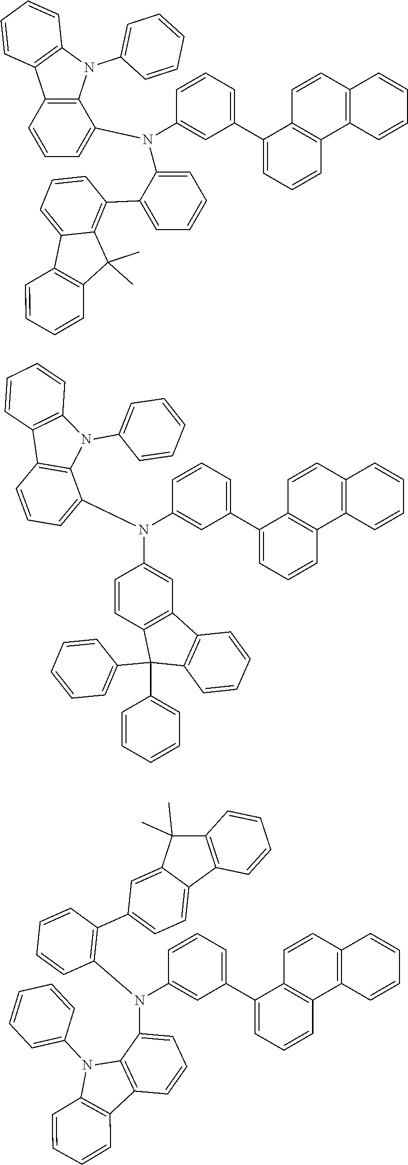

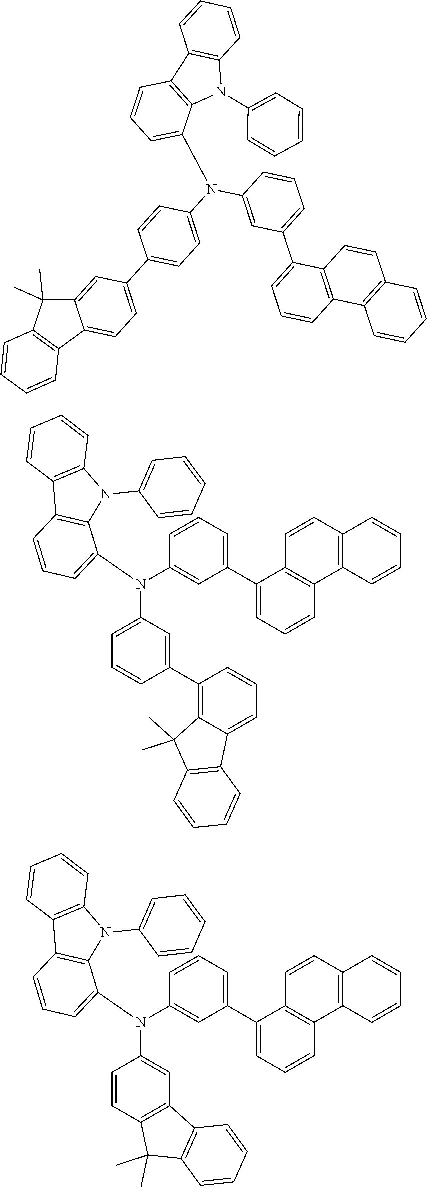

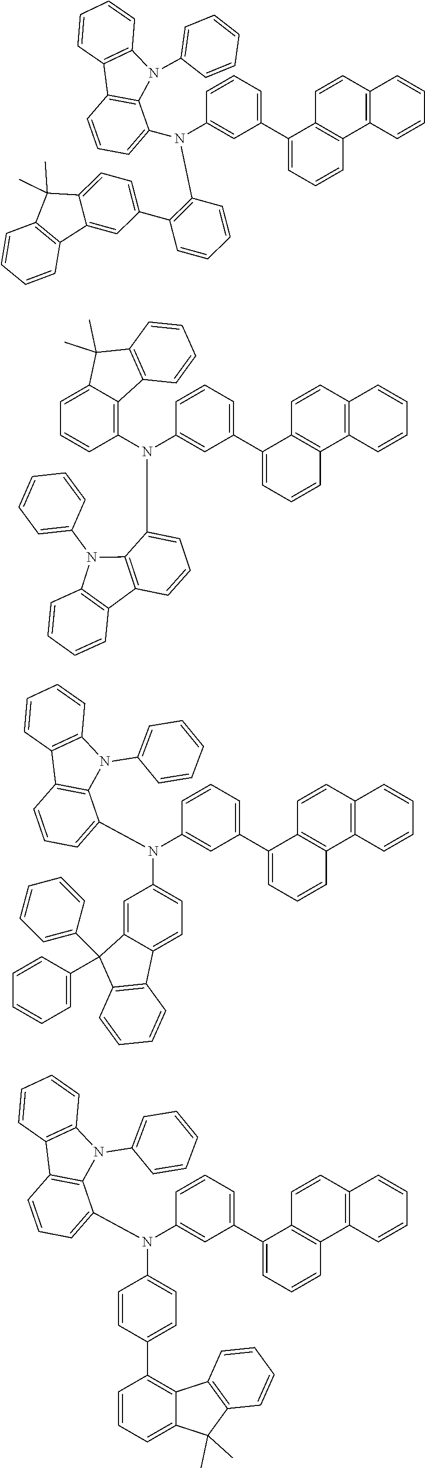

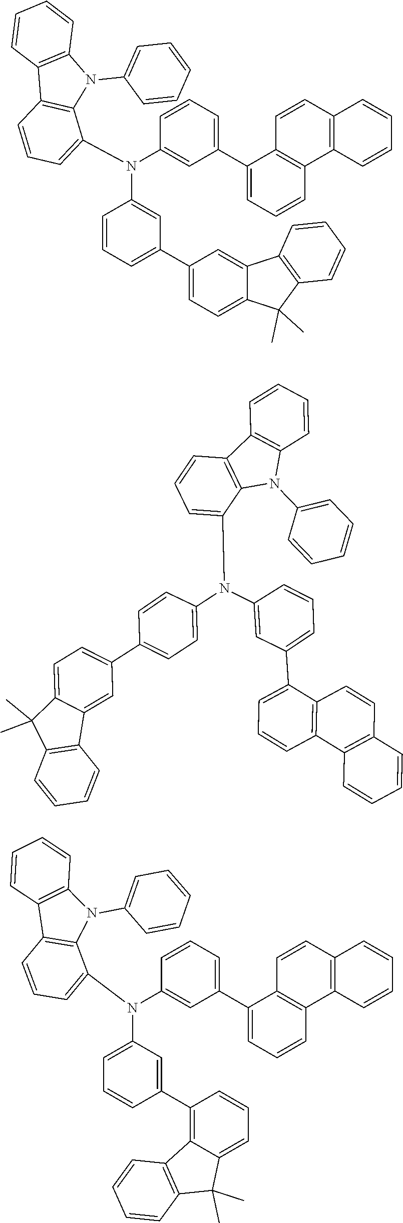

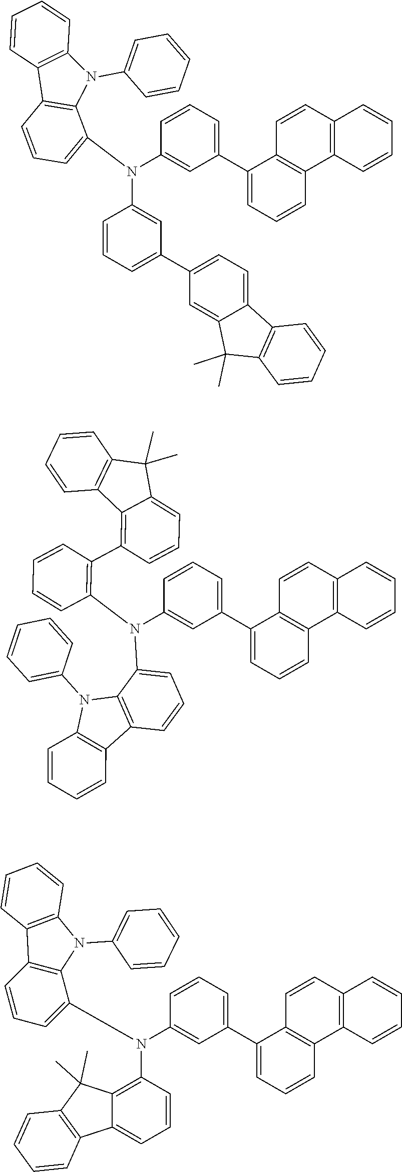 [SMILES: CC1(C)c2ccccc2-c2ccc(-c3cccc(N(c4cccc(-c5cccc6c5ccc5ccccc56)c4)c4cccc5c6ccccc6n(-c6ccccc6)c45)c3)cc21 ; CC1(C)c2ccccc2-c2c(-c3ccccc3N(c3cccc(-c4cccc5c4ccc4ccccc45)c3)c3cccc4c5ccccc5n(-c5ccccc5)c34)cccc21 ; CC1(C)c2ccccc2-c2cccc(N(c3cccc(-c4cccc5c4ccc4ccccc45)c3)c3cccc4c5ccccc5n(-c5ccccc5)c34)c21]

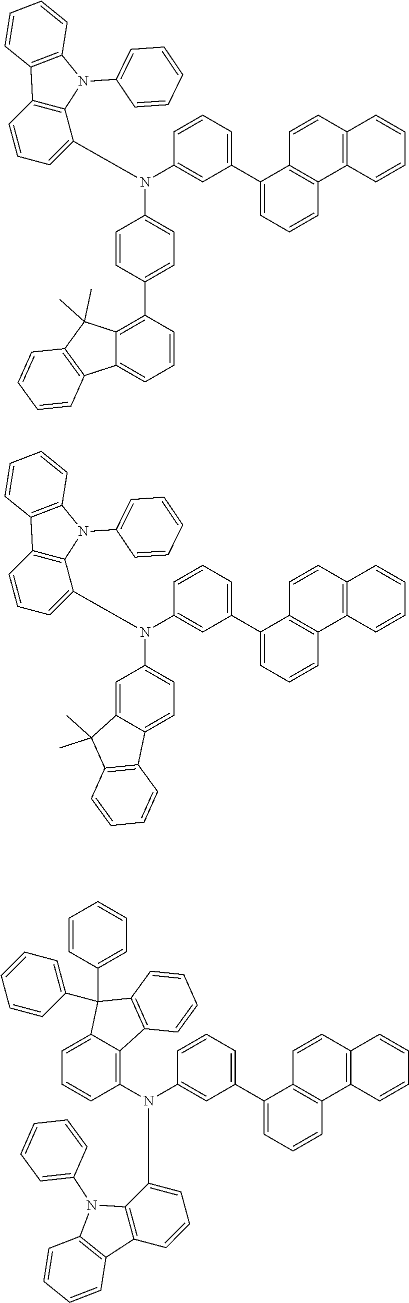

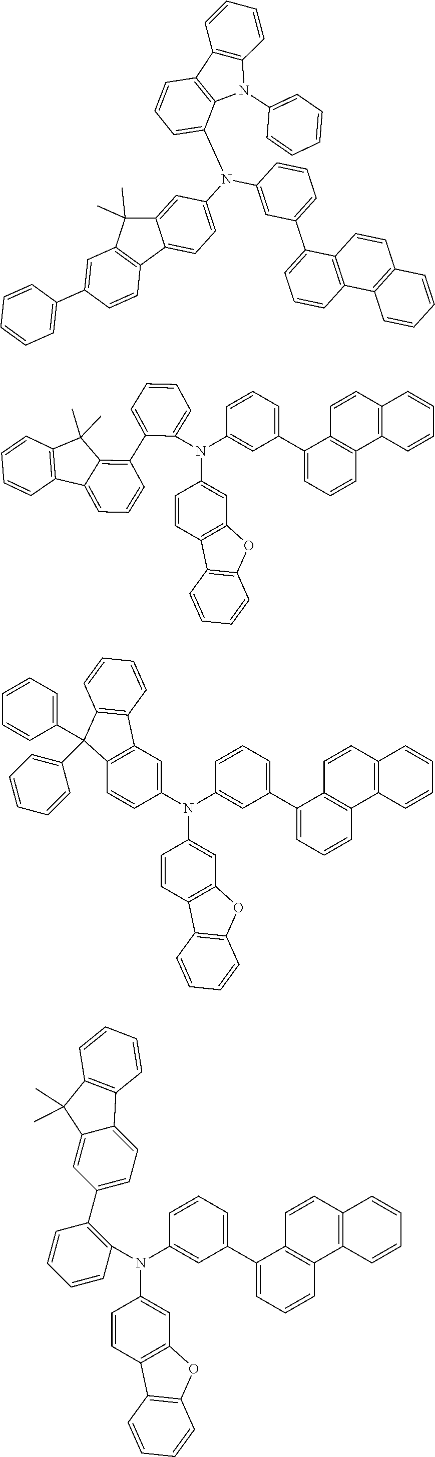 [SMILES: CC1(C)c2cc(-c3ccccc3)ccc2-c2ccc(N(c3cccc(-c4cccc5c4ccc4ccccc45)c3)c3cccc4c5ccccc5n(-c5ccccc5)c34)cc21 ; CC1(C)c2ccccc2-c2cccc(-c3ccccc3N(c3cccc(-c4cccc5c4ccc4ccccc45)c3)c3ccc4c(c3)oc3ccccc34)c21 ; c1ccc(C2(c3ccccc3)c3ccccc3-c3cc(N(c4cccc(-c5cccc6c5ccc5ccccc56)c4)c4ccc5c(c4)oc4ccccc45)ccc32)cc1 ; CC1(C)c2ccccc2-c2ccc(-c3ccccc3N(c3cccc(-c4cccc5c4ccc4ccccc45)c3)c3ccc4c(c3)oc3ccccc34)cc21]

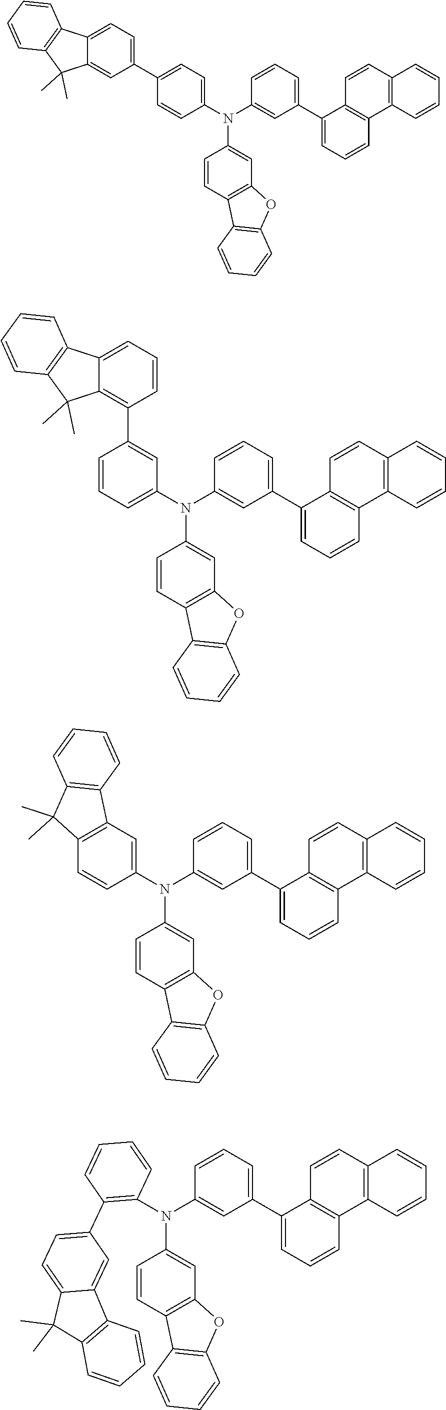 [SMILES: CC1(C)c2ccccc2-c2ccc(-c3ccc(N(c4cccc(-c5cccc6c5ccc5ccccc56)c4)c4ccc5c(c4)oc4ccccc45)cc3)cc21 ; CC1(C)c2ccccc2-c2cccc(-c3cccc(N(c4cccc(-c5cccc6c5ccc5ccccc56)c4)c4ccc5c(c4)oc4ccccc45)c3)c21 ; CC1(C)c2ccccc2-c2cc(N(c3cccc(-c4cccc5c4ccc4ccccc45)c3)c3ccc4c(c3)oc3ccccc34)ccc21 ; CC1(C)c2ccccc2-c2cc(-c3ccccc3N(c3cccc(-c4cccc5c4ccc4ccccc45)c3)c3ccc4c(c3)oc3ccccc34)ccc21]

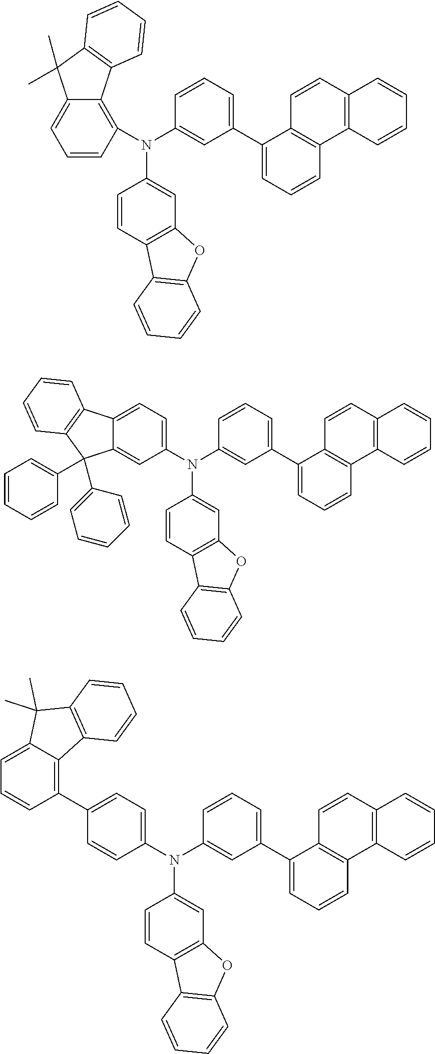 [SMILES: CC1(C)c2ccccc2-c2c(N(c3cccc(-c4cccc5c4ccc4ccccc45)c3)c3ccc4c(c3)oc3ccccc34)cccc21 ; c1ccc(C2(c3ccccc3)c3ccccc3-c3ccc(N(c4cccc(-c5cccc6c5ccc5ccccc56)c4)c4ccc5c(c4)oc4ccccc45)cc32)cc1 ; CC1(C)c2ccccc2-c2c(-c3ccc(N(c4cccc(-c5cccc6c5ccc5ccccc56)c4)c4ccc5c(c4)oc4ccccc45)cc3)cccc21]

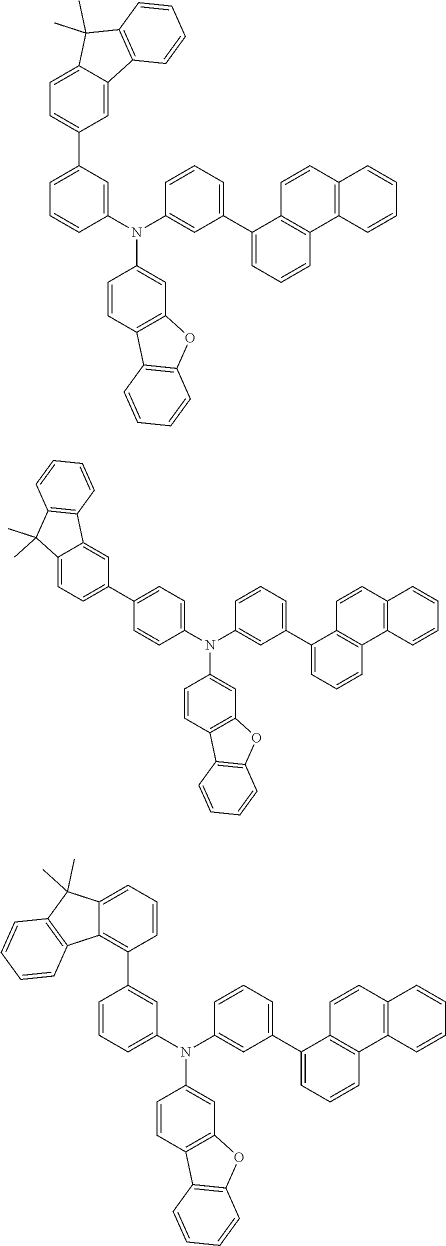 [SMILES: CC1(C)c2ccccc2-c2cc(-c3cccc(N(c4cccc(-c5cccc6c5ccc5ccccc56)c4)c4ccc5c(c4)oc4ccccc45)c3)ccc21 ; CC1(C)c2ccccc2-c2cc(-c3ccc(N(c4cccc(-c5cccc6c5ccc5ccccc56)c4)c4ccc5c(c4)oc4ccccc45)cc3)ccc21 ; CC1(C)c2ccccc2-c2c(-c3cccc(N(c4cccc(-c5cccc6c5ccc5ccccc56)c4)c4ccc5c(c4)oc4ccccc45)c3)cccc21]

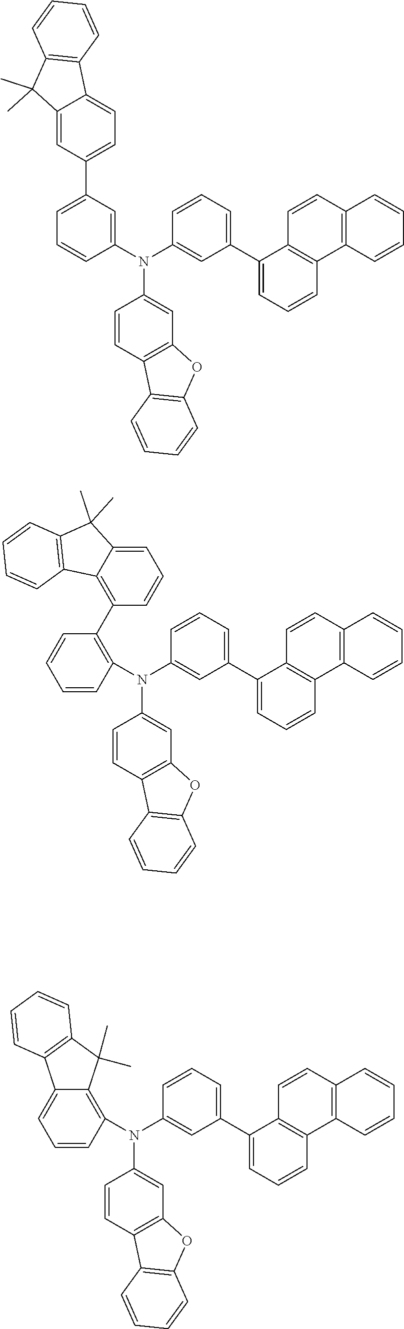 [SMILES: CC1(C)c2ccccc2-c2ccc(-c3cccc(N(c4cccc(-c5cccc6c5ccc5ccccc56)c4)c4ccc5c(c4)oc4ccccc45)c3)cc21 ; CC1(C)c2ccccc2-c2c(-c3ccccc3N(c3cccc(-c4cccc5c4ccc4ccccc45)c3)c3ccc4c(c3)oc3ccccc34)cccc21 ; CC1(C)c2ccccc2-c2cccc(N(c3cccc(-c4cccc5c4ccc4ccccc45)c3)c3ccc4c(c3)oc3ccccc34)c21]

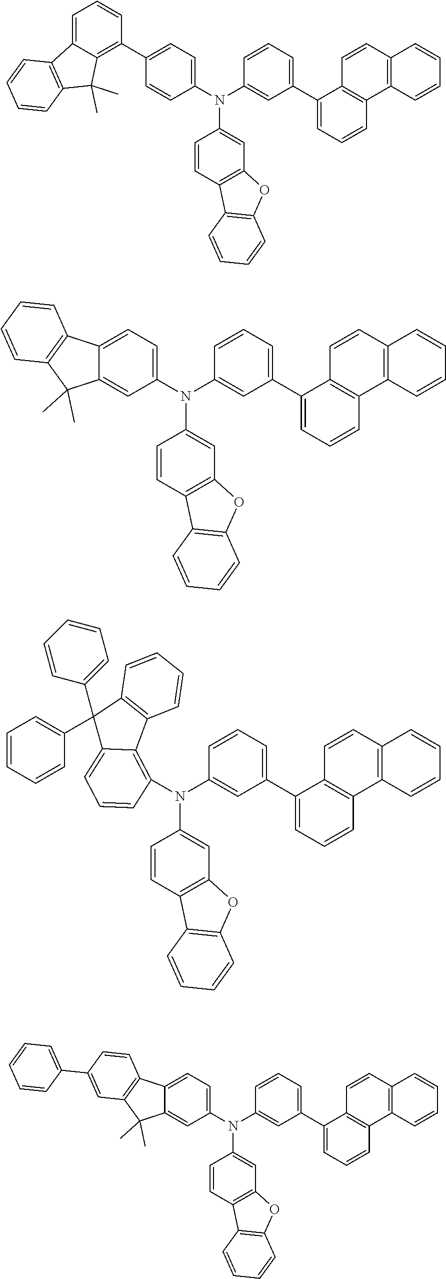 [SMILES: CC1(C)c2ccccc2-c2cccc(-c3ccc(N(c4cccc(-c5cccc6c5ccc5ccccc56)c4)c4ccc5c(c4)oc4ccccc45)cc3)c21 ; CC1(C)c2ccccc2-c2ccc(N(c3cccc(-c4cccc5c4ccc4ccccc45)c3)c3ccc4c(c3)oc3ccccc34)cc21 ; c1ccc(C2(c3ccccc3)c3ccccc3-c3c(N(c4cccc(-c5cccc6c5ccc5ccccc56)c4)c4ccc5c(c4)oc4ccccc45)cccc32)cc1 ; CC1(C)c2cc(-c3ccccc3)ccc2-c2ccc(N(c3cccc(-c4cccc5c4ccc4ccccc45)c3)c3ccc4c(c3)oc3ccccc34)cc21]

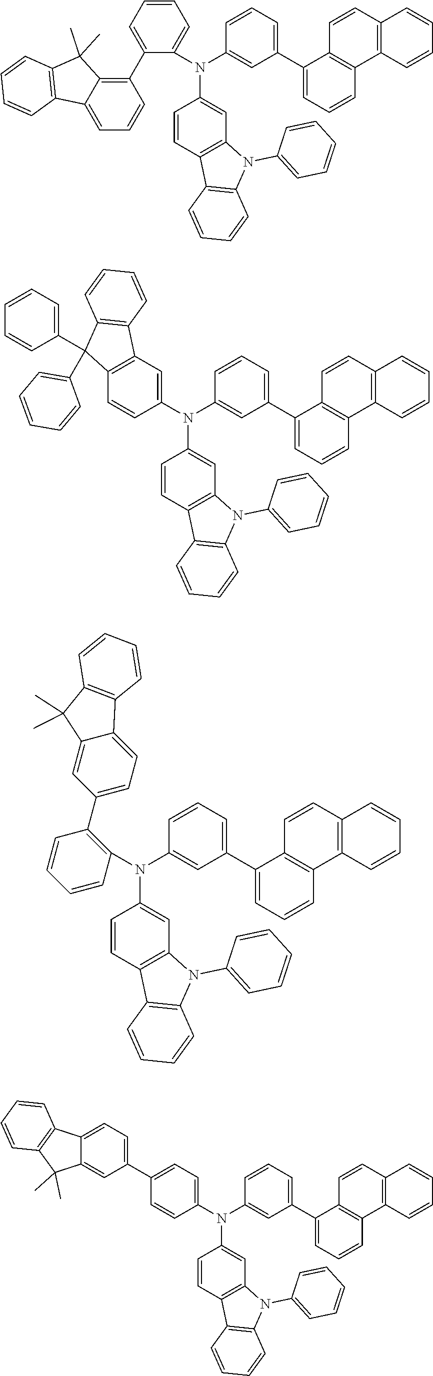 [SMILES: CC1(C)c2ccccc2-c2cccc(-c3ccccc3N(c3cccc(-c4cccc5c4ccc4ccccc45)c3)c3ccc4c5ccccc5n(-c5ccccc5)c4c3)c21 ; c1ccc(-n2c3ccccc3c3ccc(N(c4cccc(-c5cccc6c5ccc5ccccc56)c4)c4ccc5c(c4)-c4ccccc4C5(c4ccccc4)c4ccccc4)cc32)cc1 ; CC1(C)c2ccccc2-c2ccc(-c3ccccc3N(c3cccc(-c4cccc5c4ccc4ccccc45)c3)c3ccc4c5ccccc5n(-c5ccccc5)c4c3)cc21 ; CC1(C)c2ccccc2-c2ccc(-c3ccc(N(c4cccc(-c5cccc6c5ccc5ccccc56)c4)c4ccc5c6ccccc6n(-c6ccccc6)c5c4)cc3)cc21]

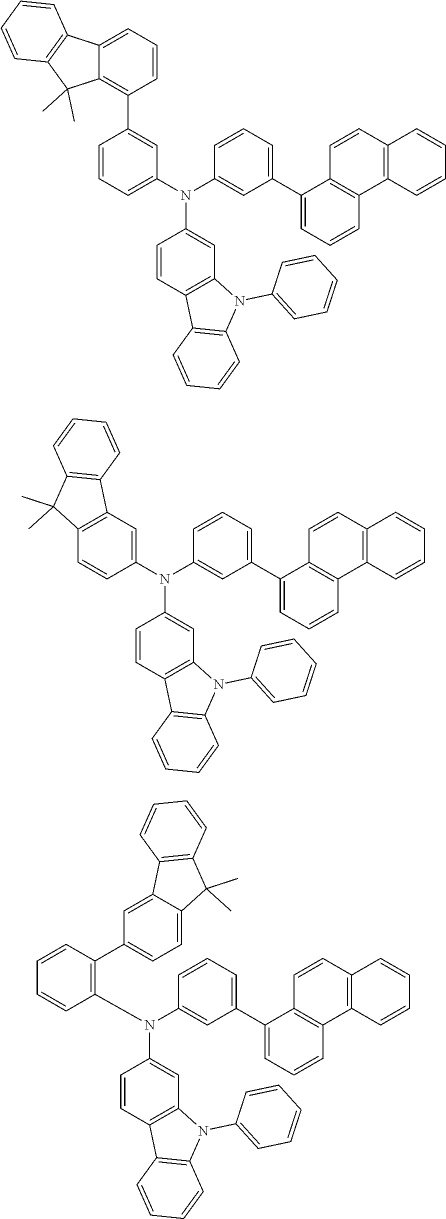

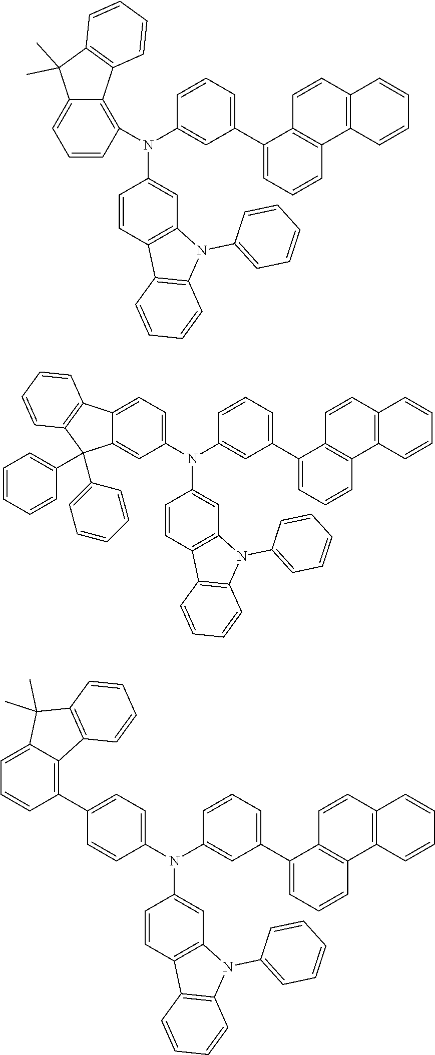 [SMILES: CC1(C)c2ccccc2-c2c(N(c3cccc(-c4cccc5c4ccc4ccccc45)c3)c3ccc4c5ccccc5n(-c5ccccc5)c4c3)cccc21 ; c1ccc(-n2c3ccccc3c3ccc(N(c4cccc(-c5cccc6c5ccc5ccccc56)c4)c4ccc5c(c4)C(c4ccccc4)(c4ccccc4)c4ccccc4-5)cc32)cc1 ; CC1(C)c2ccccc2-c2c(-c3ccc(N(c4cccc(-c5cccc6c5ccc5ccccc56)c4)c4ccc5c6ccccc6n(-c6ccccc6)c5c4)cc3)cccc21]

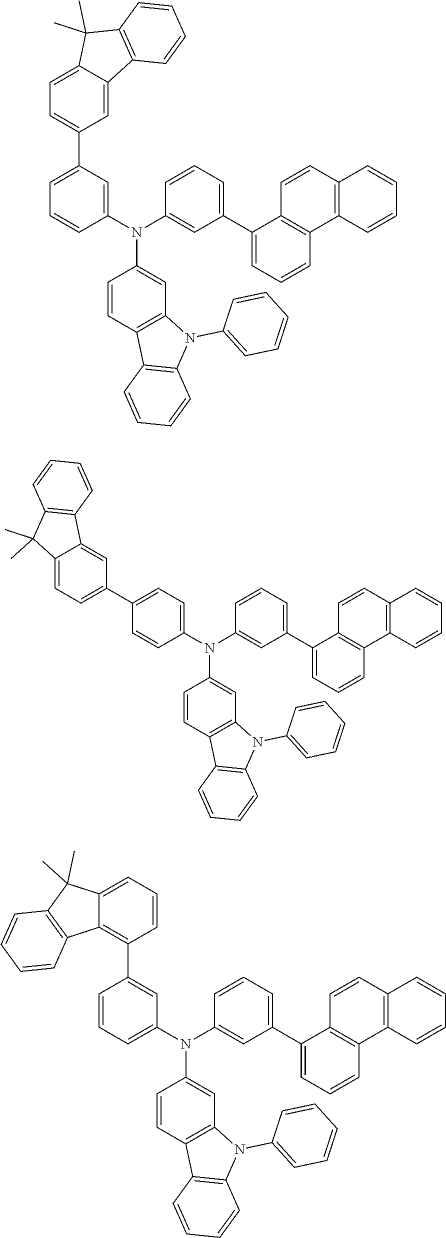 [SMILES: CC1(C)c2ccccc2-c2cc(-c3cccc(N(c4cccc(-c5cccc6c5ccc5ccccc56)c4)c4ccc5c6ccccc6n(-c6ccccc6)c5c4)c3)ccc21 ; CC1(C)c2ccccc2-c2cc(-c3ccc(N(c4cccc(-c5cccc6c5ccc5ccccc56)c4)c4ccc5c6ccccc6n(-c6ccccc6)c5c4)cc3)ccc21 ; CC1(C)c2ccccc2-c2c(-c3cccc(N(c4cccc(-c5cccc6c5ccc5ccccc56)c4)c4ccc5c6ccccc6n(-c6ccccc6)c5c4)c3)cccc21]

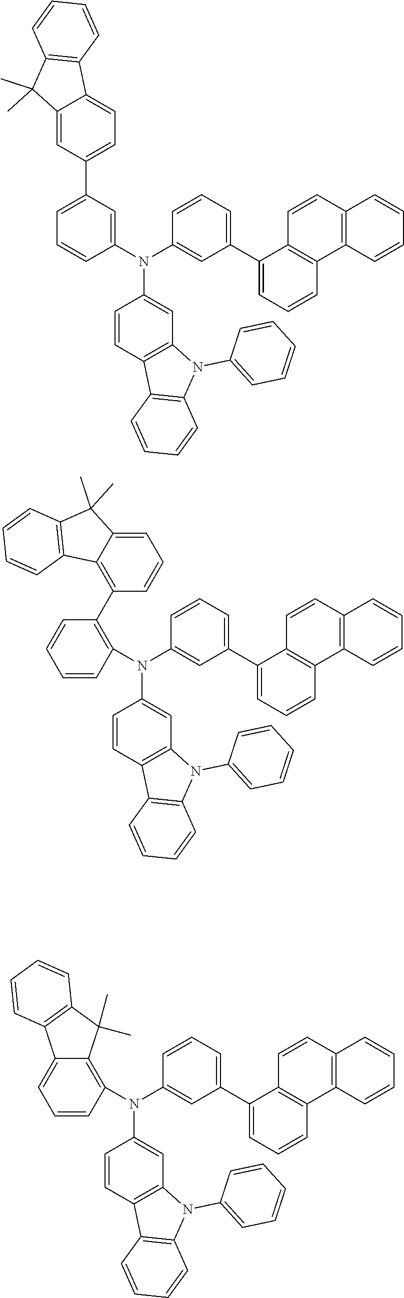

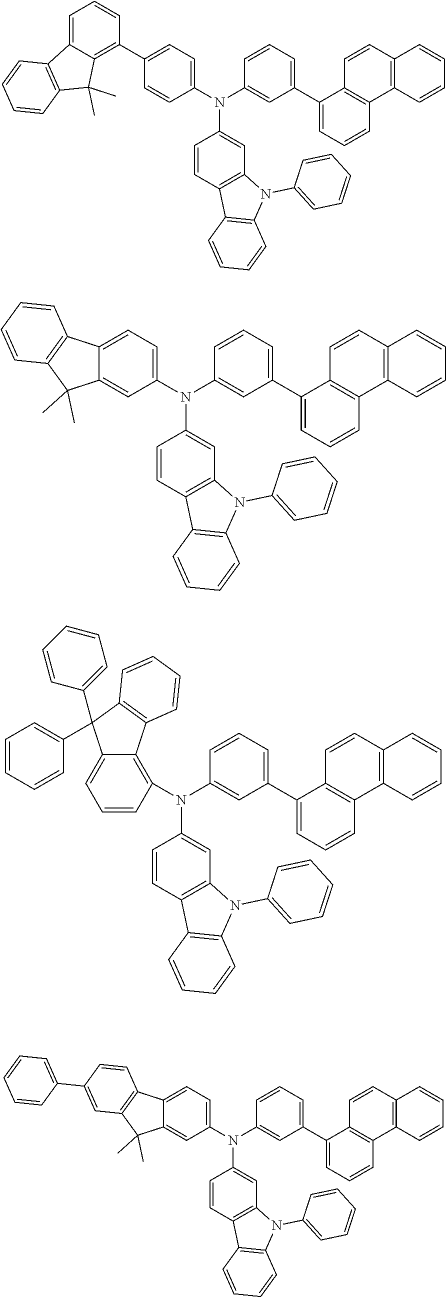 [SMILES: CC1(C)c2ccccc2-c2cccc(-c3ccc(N(c4cccc(-c5cccc6c5ccc5ccccc56)c4)c4ccc5c6ccccc6n(-c6ccccc6)c5c4)cc3)c21 ; CC1(C)c2ccccc2-c2ccc(N(c3cccc(-c4cccc5c4ccc4ccccc45)c3)c3ccc4c5ccccc5n(-c5ccccc5)c4c3)cc21 ; c1ccc(-n2c3ccccc3c3ccc(N(c4cccc(-c5cccc6c5ccc5ccccc56)c4)c4cccc5c4-c4ccccc4C5(c4ccccc4)c4ccccc4)cc32)cc1 ; CC1(C)c2cc(-c3ccccc3)ccc2-c2ccc(N(c3cccc(-c4cccc5c4ccc4ccccc45)c3)c3ccc4c5ccccc5n(-c5ccccc5)c4c3)cc21]

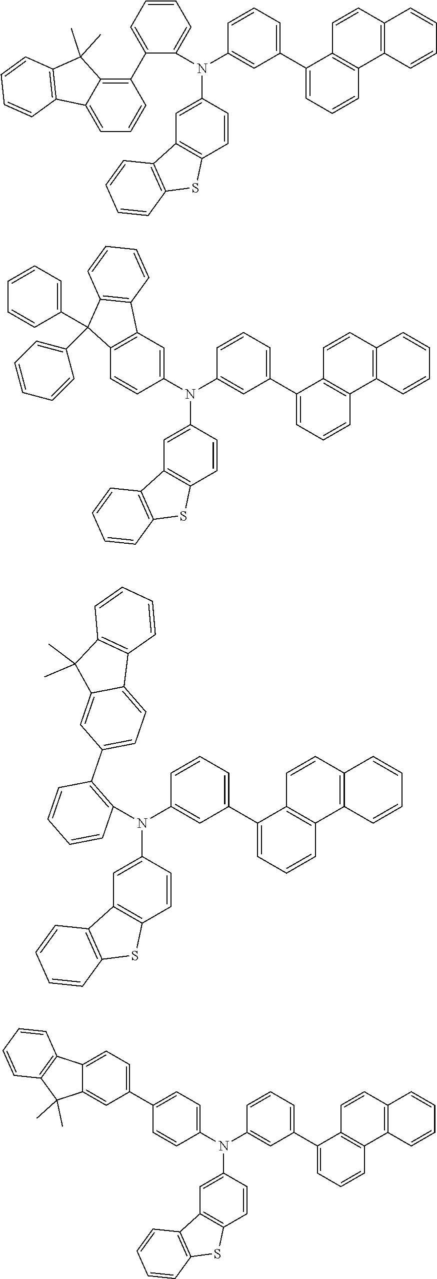

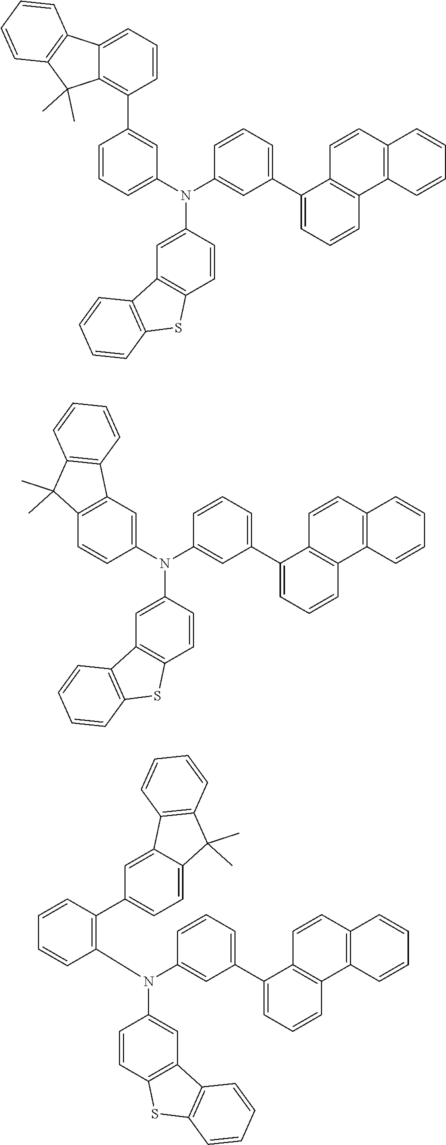 [SMILES: CC1(C)c2ccccc2-c2cccc(-c3cccc(N(c4cccc(-c5cccc6c5ccc5ccccc56)c4)c4ccc5sc6ccccc6c5c4)c3)c21 ; CC1(C)c2ccccc2-c2cc(N(c3cccc(-c4cccc5c4ccc4ccccc45)c3)c3ccc4sc5ccccc5c4c3)ccc21 ; CC1(C)c2ccccc2-c2cc(-c3ccccc3N(c3cccc(-c4cccc5c4ccc4ccccc45)c3)c3ccc4sc5ccccc5c4c3)ccc21]

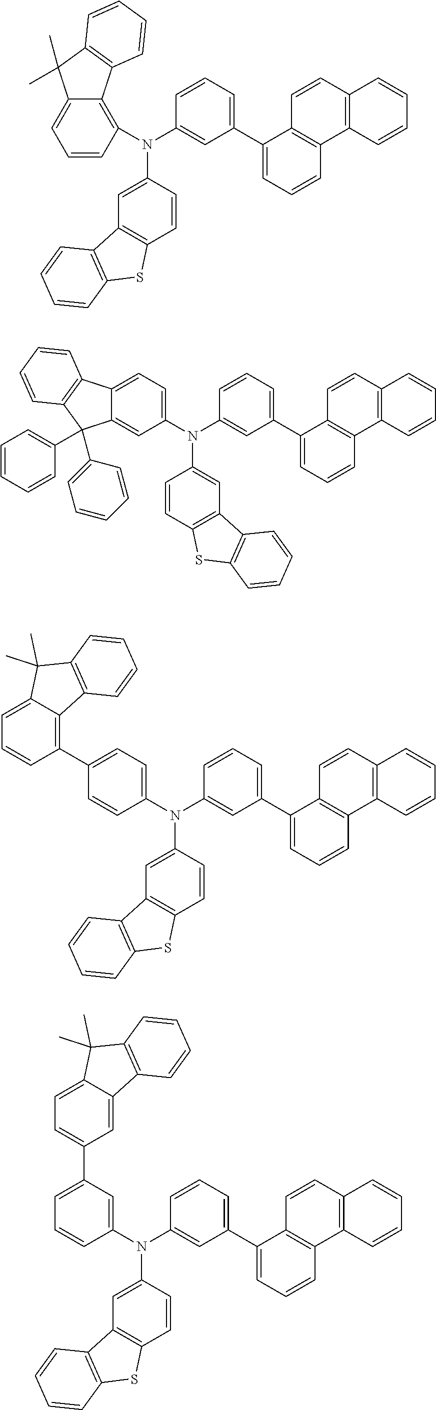

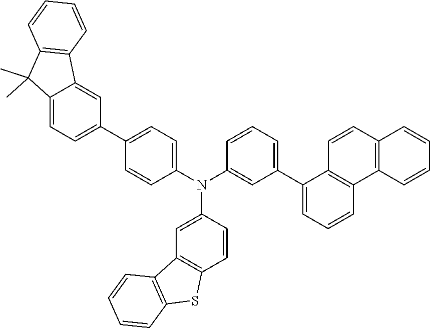 [SMILES: CC1(C)c2ccccc2-c2cc(-c3ccc(N(c4cccc(-c5cccc6c5ccc5ccccc56)c4)c4ccc5sc6ccccc6c5c4)cc3)ccc21]

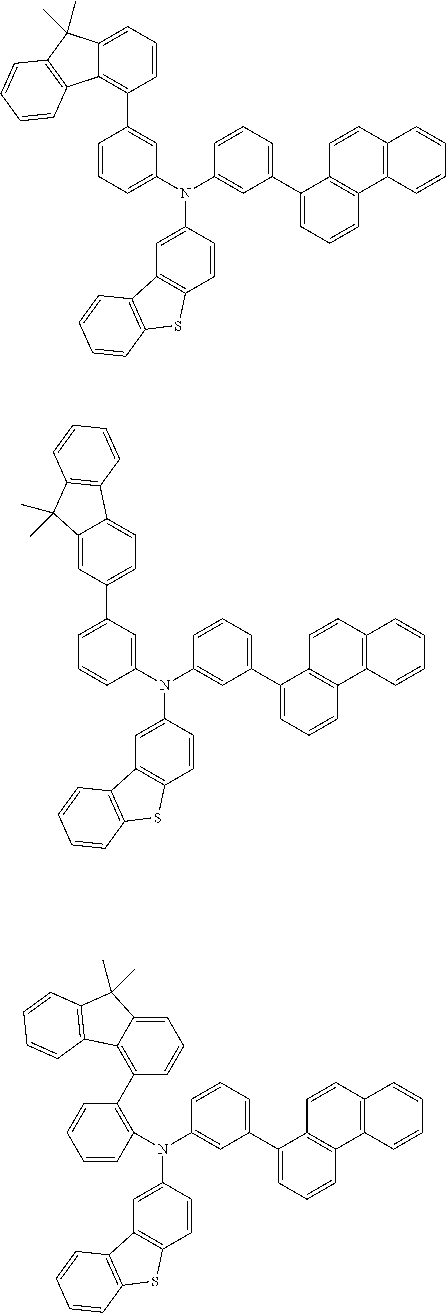 [SMILES: CC1(C)c2ccccc2-c2c(-c3cccc(N(c4cccc(-c5cccc6c5ccc5ccccc56)c4)c4ccc5sc6ccccc6c5c4)c3)cccc21 ; CC1(C)c2ccccc2-c2ccc(-c3cccc(N(c4cccc(-c5cccc6c5ccc5ccccc56)c4)c4ccc5sc6ccccc6c5c4)c3)cc21 ; CC1(C)c2ccccc2-c2c(-c3ccccc3N(c3cccc(-c4cccc5c4ccc4ccccc45)c3)c3ccc4sc5ccccc5c4c3)cccc21]

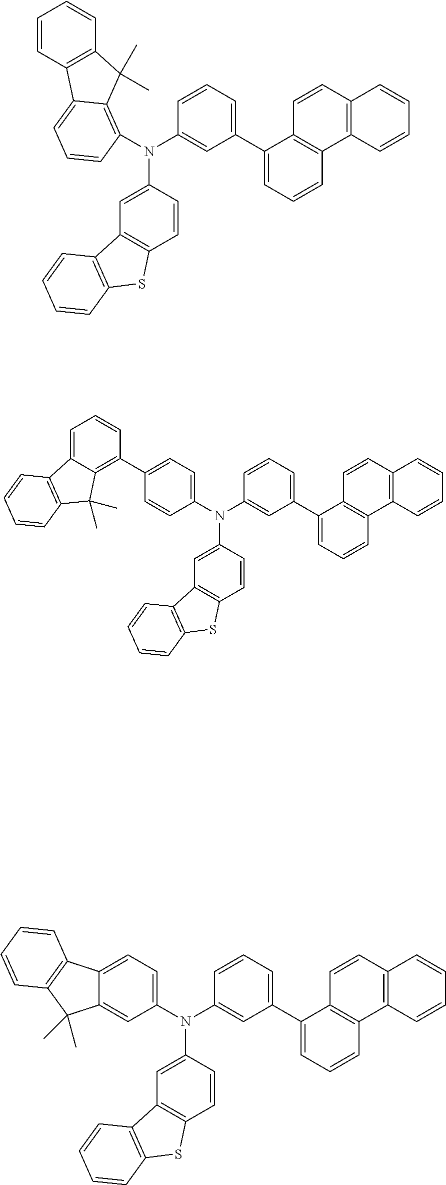 [SMILES: CC1(C)c2ccccc2-c2cccc(N(c3cccc(-c4cccc5c4ccc4ccccc45)c3)c3ccc4sc5ccccc5c4c3)c21 ; CC1(C)c2ccccc2-c2cccc(-c3ccc(N(c4cccc(-c5cccc6c5ccc5ccccc56)c4)c4ccc5sc6ccccc6c5c4)cc3)c21 ; CC1(C)c2ccccc2-c2ccc(N(c3cccc(-c4cccc5c4ccc4ccccc45)c3)c3ccc4sc5ccccc5c4c3)cc21]

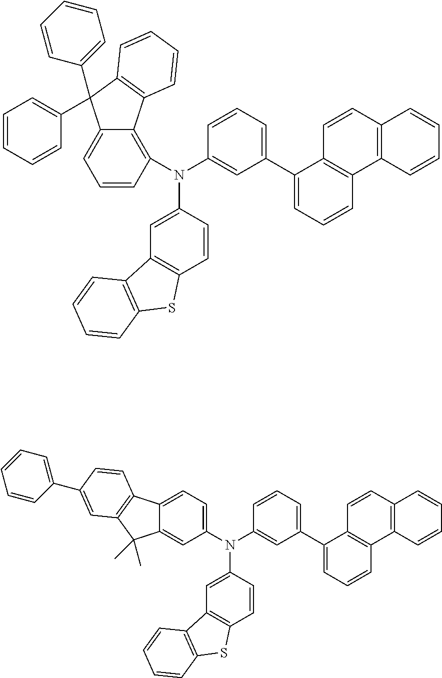 [SMILES: c1ccc(C2(c3ccccc3)c3ccccc3-c3c(N(c4cccc(-c5cccc6c5ccc5ccccc56)c4)c4ccc5sc6ccccc6c5c4)cccc32)cc1 ; CC1(C)c2cc(-c3ccccc3)ccc2-c2ccc(N(c3cccc(-c4cccc5c4ccc4ccccc45)c3)c3ccc4sc5ccccc5c4c3)cc21]

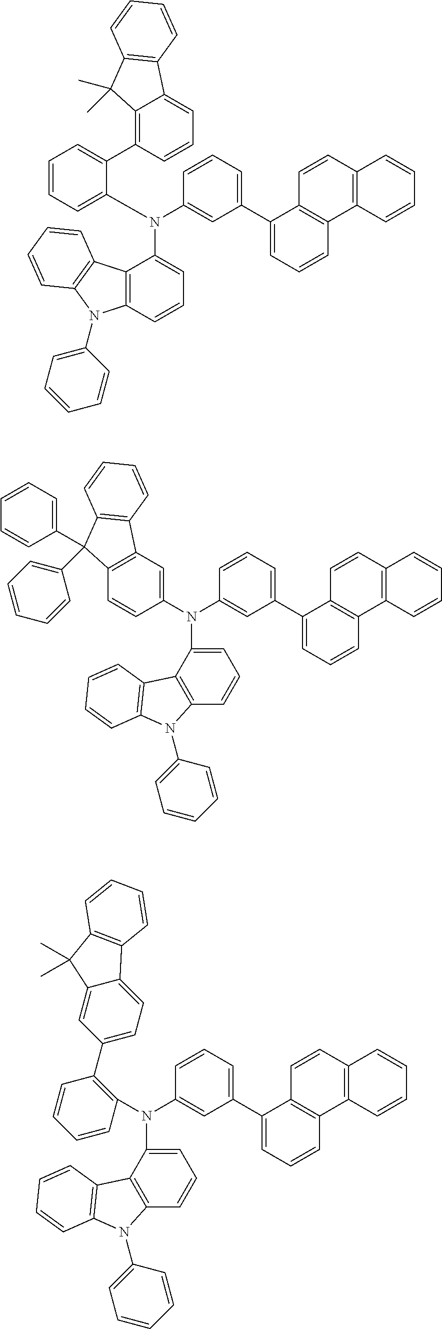 [SMILES: CC1(C)c2ccccc2-c2cccc(-c3ccccc3N(c3cccc(-c4cccc5c4ccc4ccccc45)c3)c3cccc4c3c3ccccc3n4-c3ccccc3)c21 ; c1ccc(-n2c3ccccc3c3c(N(c4cccc(-c5cccc6c5ccc5ccccc56)c4)c4ccc5c(c4)-c4ccccc4C5(c4ccccc4)c4ccccc4)cccc32)cc1 ; CC1(C)c2ccccc2-c2ccc(-c3ccccc3N(c3cccc(-c4cccc5c4ccc4ccccc45)c3)c3cccc4c3c3ccccc3n4-c3ccccc3)cc21]

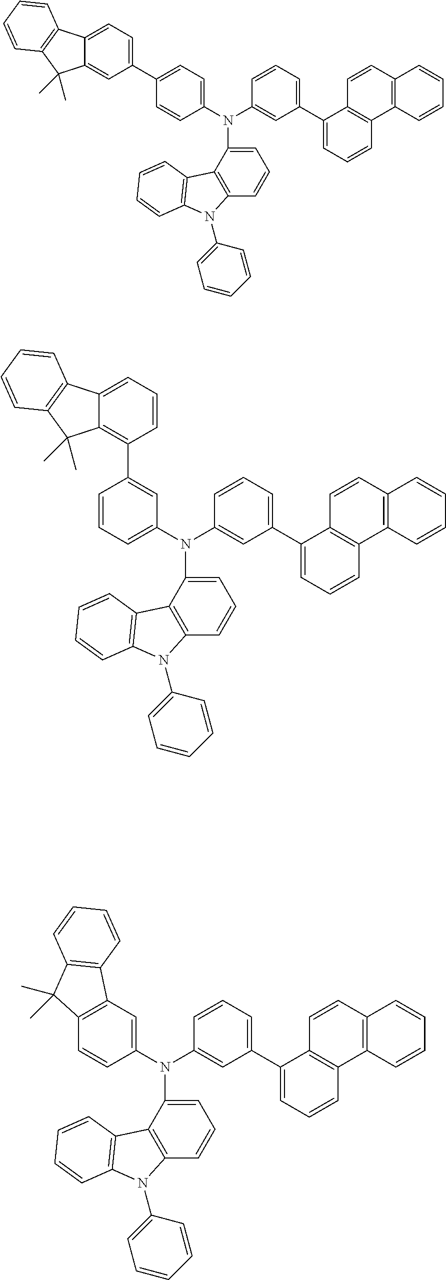 [SMILES: CC1(C)c2ccccc2-c2ccc(-c3ccc(N(c4cccc(-c5cccc6c5ccc5ccccc56)c4)c4cccc5c4c4ccccc4n5-c4ccccc4)cc3)cc21 ; CC1(C)c2ccccc2-c2cccc(-c3cccc(N(c4cccc(-c5cccc6c5ccc5ccccc56)c4)c4cccc5c4c4ccccc4n5-c4ccccc4)c3)c21 ; CC1(C)c2ccccc2-c2cc(N(c3cccc(-c4cccc5c4ccc4ccccc45)c3)c3cccc4c3c3ccccc3n4-c3ccccc3)ccc21]

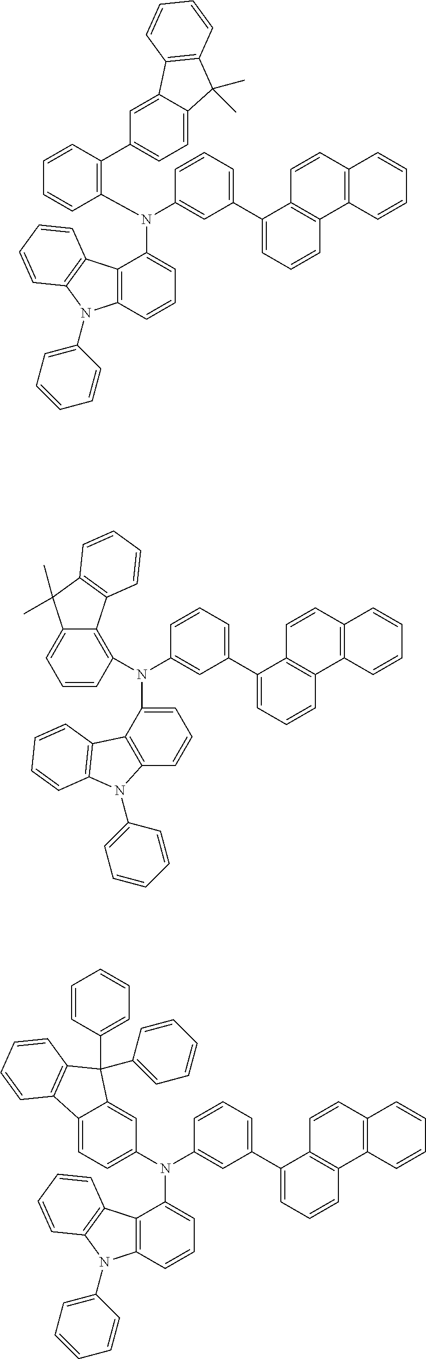 [SMILES: CC1(C)c2ccccc2-c2cc(-c3ccccc3N(c3cccc(-c4cccc5c4ccc4ccccc45)c3)c3cccc4c3c3ccccc3n4-c3ccccc3)ccc21 ; CC1(C)c2ccccc2-c2c(N(c3cccc(-c4cccc5c4ccc4ccccc45)c3)c3cccc4c3c3ccccc3n4-c3ccccc3)cccc21 ; c1ccc(-n2c3ccccc3c3c(N(c4cccc(-c5cccc6c5ccc5ccccc56)c4)c4ccc5c(c4)C(c4ccccc4)(c4ccccc4)c4ccccc4-5)cccc32)cc1]

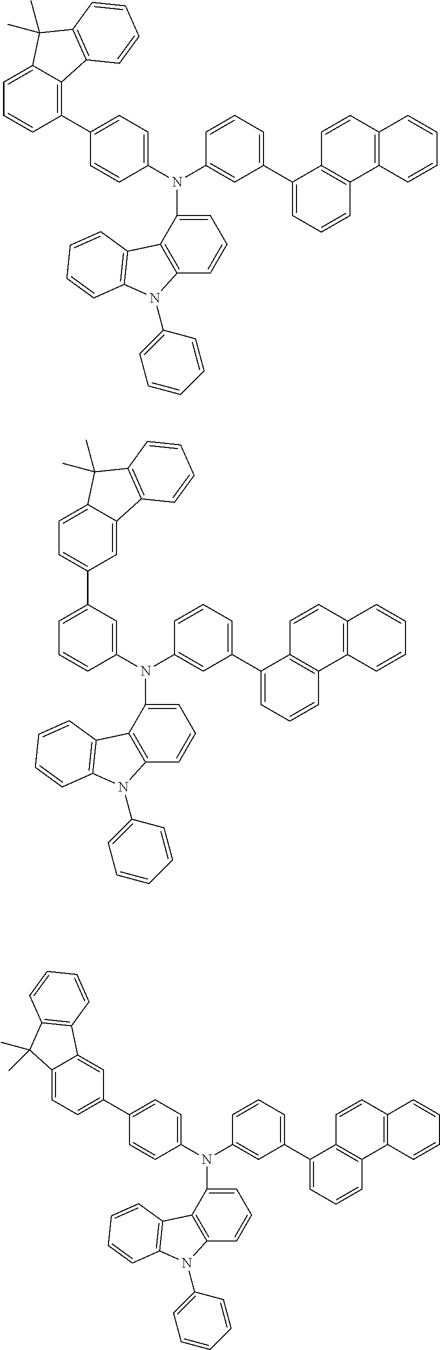 [SMILES: CC1(C)c2ccccc2-c2c(-c3ccc(N(c4cccc(-c5cccc6c5ccc5ccccc56)c4)c4cccc5c4c4ccccc4n5-c4ccccc4)cc3)cccc21 ; CC1(C)c2ccccc2-c2cc(-c3cccc(N(c4cccc(-c5cccc6c5ccc5ccccc56)c4)c4cccc5c4c4ccccc4n5-c4ccccc4)c3)ccc21 ; CC1(C)c2ccccc2-c2cc(-c3ccc(N(c4cccc(-c5cccc6c5ccc5ccccc56)c4)c4cccc5c4c4ccccc4n5-c4ccccc4)cc3)ccc21]

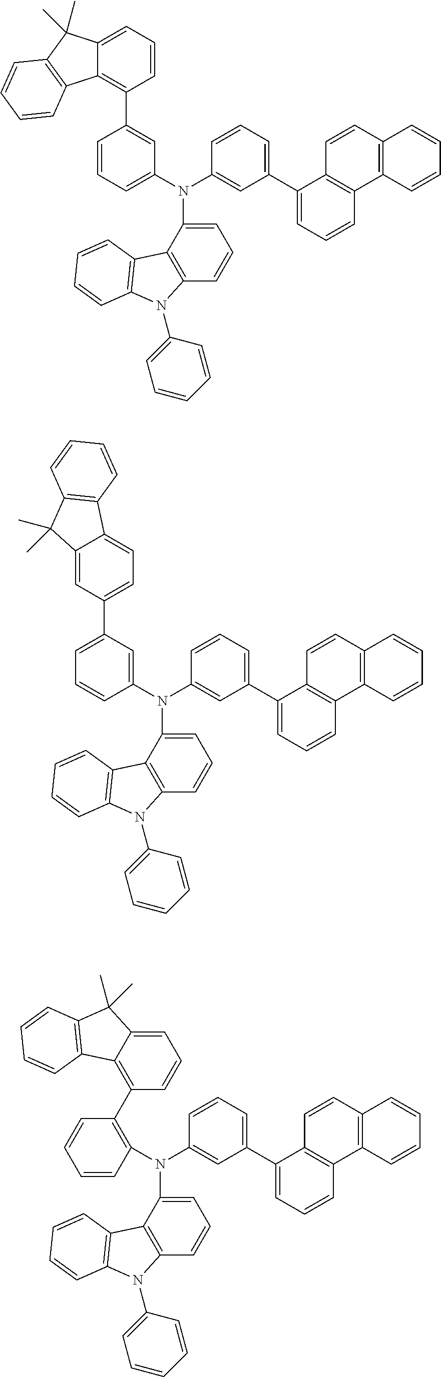 [SMILES: CC1(C)c2ccccc2-c2c(-c3cccc(N(c4cccc(-c5cccc6c5ccc5ccccc56)c4)c4cccc5c4c4ccccc4n5-c4ccccc4)c3)cccc21 ; CC1(C)c2ccccc2-c2ccc(-c3cccc(N(c4cccc(-c5cccc6c5ccc5ccccc56)c4)c4cccc5c4c4ccccc4n5-c4ccccc4)c3)cc21 ; CC1(C)c2ccccc2-c2c(-c3ccccc3N(c3cccc(-c4cccc5c4ccc4ccccc45)c3)c3cccc4c3c3ccccc3n4-c3ccccc3)cccc21]

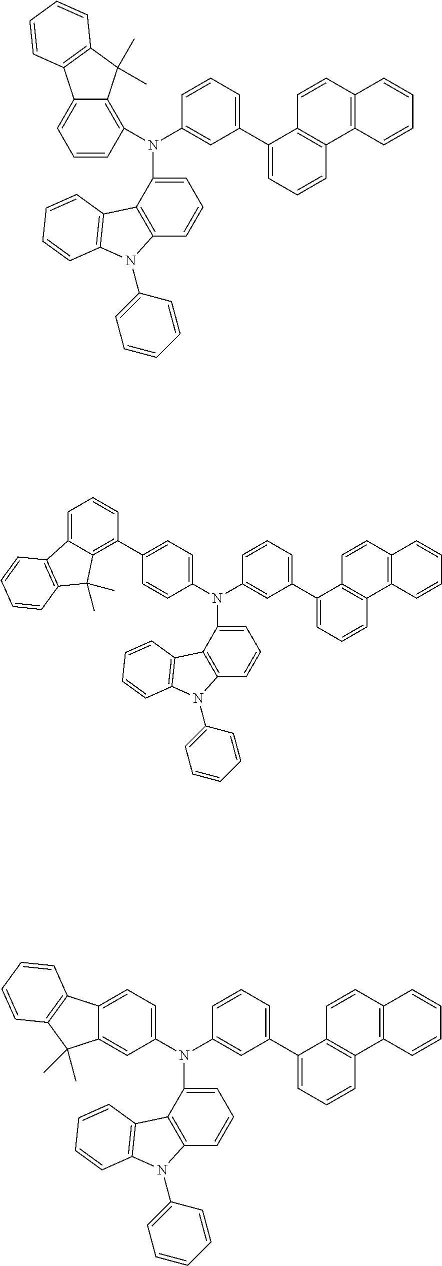 [SMILES: CC1(C)c2ccccc2-c2cccc(N(c3cccc(-c4cccc5c4ccc4ccccc45)c3)c3cccc4c3c3ccccc3n4-c3ccccc3)c21 ; CC1(C)c2ccccc2-c2cccc(-c3ccc(N(c4cccc(-c5cccc6c5ccc5ccccc56)c4)c4cccc5c4c4ccccc4n5-c4ccccc4)cc3)c21 ; CC1(C)c2ccccc2-c2ccc(N(c3cccc(-c4cccc5c4ccc4ccccc45)c3)c3cccc4c3c3ccccc3n4-c3ccccc3)cc21]

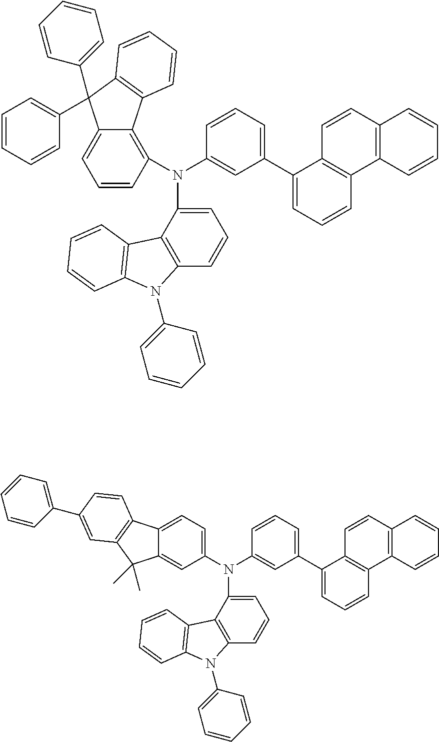 [SMILES: c1ccc(-n2c3ccccc3c3c(N(c4cccc(-c5cccc6c5ccc5ccccc56)c4)c4cccc5c4-c4ccccc4C5(c4ccccc4)c4ccccc4)cccc32)cc1 ; CC1(C)c2cc(-c3ccccc3)ccc2-c2ccc(N(c3cccc(-c4cccc5c4ccc4ccccc45)c3)c3cccc4c3c3ccccc3n4-c3ccccc3)cc21]

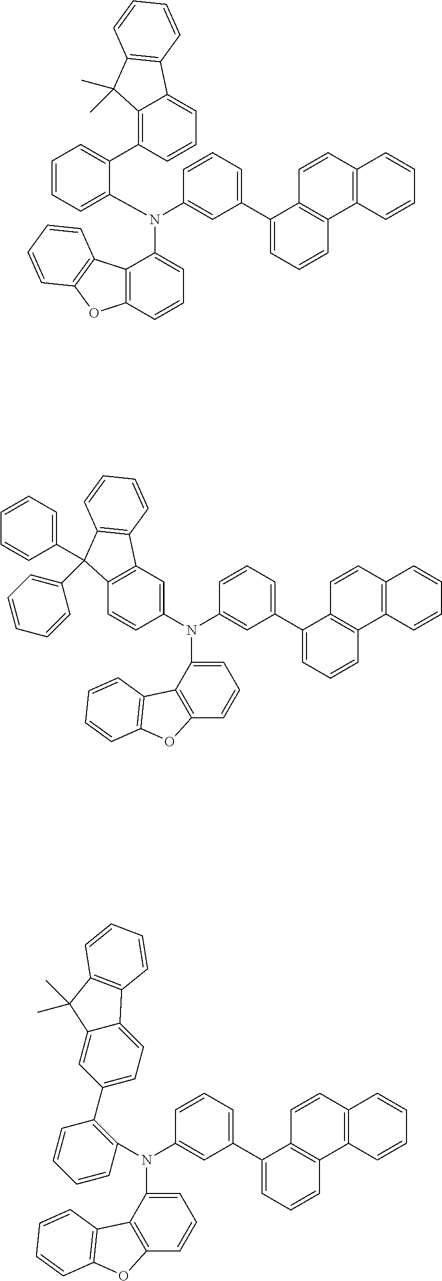 [SMILES: CC1(C)c2ccccc2-c2cccc(-c3ccccc3N(c3cccc(-c4cccc5c4ccc4ccccc45)c3)c3cccc4oc5ccccc5c34)c21 ; c1ccc(C2(c3ccccc3)c3ccccc3-c3cc(N(c4cccc(-c5cccc6c5ccc5ccccc56)c4)c4cccc5oc6ccccc6c45)ccc32)cc1 ; CC1(C)c2ccccc2-c2ccc(-c3ccccc3N(c3cccc(-c4cccc5c4ccc4ccccc45)c3)c3cccc4oc5ccccc5c34)cc21]

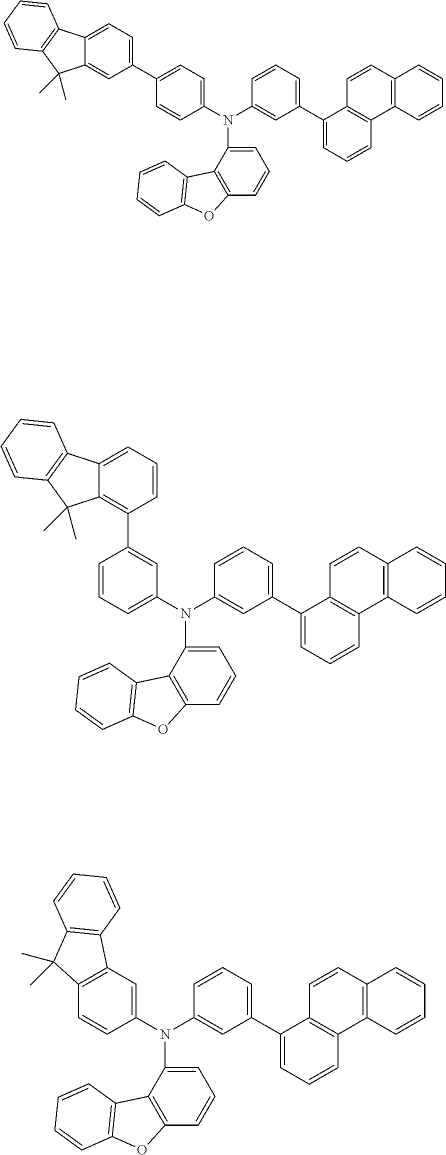 [SMILES: CC1(C)c2ccccc2-c2ccc(-c3ccc(N(c4cccc(-c5cccc6c5ccc5ccccc56)c4)c4cccc5oc6ccccc6c45)cc3)cc21 ; CC1(C)c2ccccc2-c2cccc(-c3cccc(N(c4cccc(-c5cccc6c5ccc5ccccc56)c4)c4cccc5oc6ccccc6c45)c3)c21 ; CC1(C)c2ccccc2-c2cc(N(c3cccc(-c4cccc5c4ccc4ccccc45)c3)c3cccc4oc5ccccc5c34)ccc21]

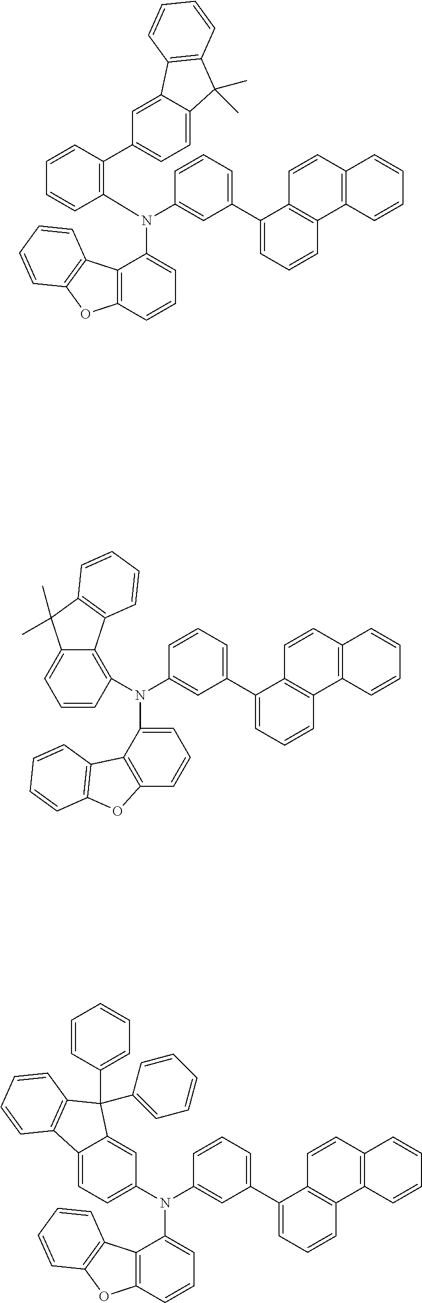 [SMILES: CC1(C)c2ccccc2-c2cc(-c3ccccc3N(c3cccc(-c4cccc5c4ccc4ccccc45)c3)c3cccc4oc5ccccc5c34)ccc21 ; CC1(C)c2ccccc2-c2c(N(c3cccc(-c4cccc5c4ccc4ccccc45)c3)c3cccc4oc5ccccc5c34)cccc21 ; c1ccc(C2(c3ccccc3)c3ccccc3-c3ccc(N(c4cccc(-c5cccc6c5ccc5ccccc56)c4)c4cccc5oc6ccccc6c45)cc32)cc1]

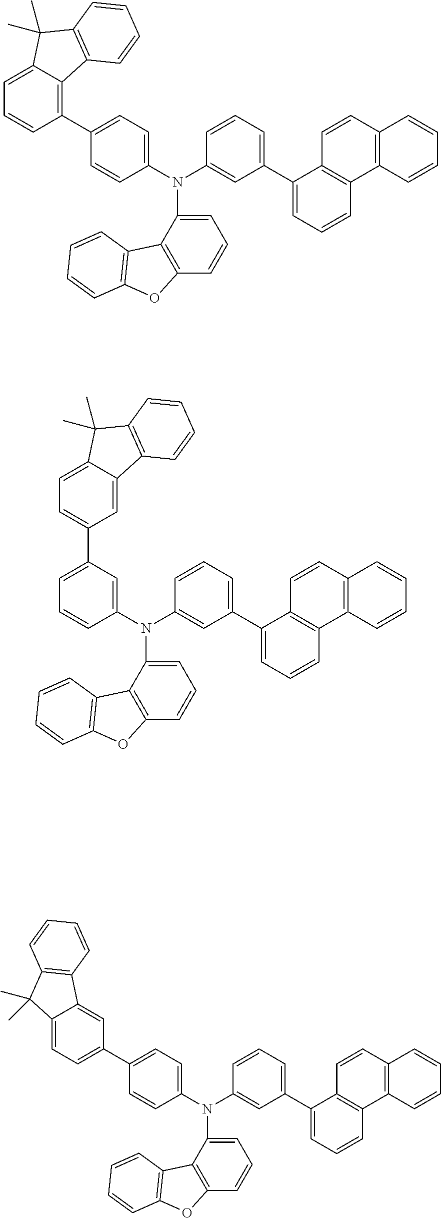 [SMILES: CC1(C)c2ccccc2-c2c(-c3ccc(N(c4cccc(-c5cccc6c5ccc5ccccc56)c4)c4cccc5oc6ccccc6c45)cc3)cccc21 ; CC1(C)c2ccccc2-c2cc(-c3cccc(N(c4cccc(-c5cccc6c5ccc5ccccc56)c4)c4cccc5oc6ccccc6c45)c3)ccc21 ; CC1(C)c2ccccc2-c2cc(-c3ccc(N(c4cccc(-c5cccc6c5ccc5ccccc56)c4)c4cccc5oc6ccccc6c45)cc3)ccc21]

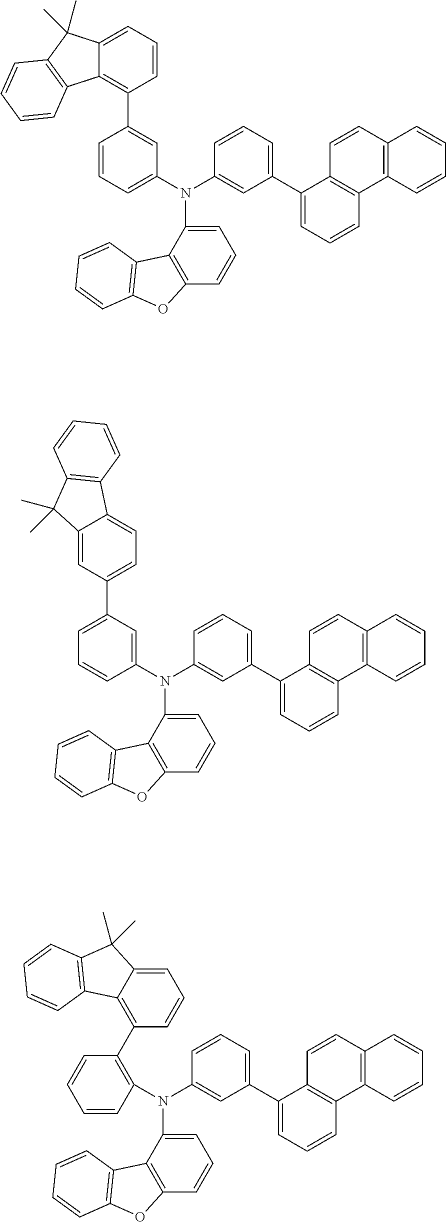 [SMILES: CC1(C)c2ccccc2-c2c(-c3cccc(N(c4cccc(-c5cccc6c5ccc5ccccc56)c4)c4cccc5oc6ccccc6c45)c3)cccc21 ; CC1(C)c2ccccc2-c2ccc(-c3cccc(N(c4cccc(-c5cccc6c5ccc5ccccc56)c4)c4cccc5oc6ccccc6c45)c3)cc21 ; CC1(C)c2ccccc2-c2c(-c3ccccc3N(c3cccc(-c4cccc5c4ccc4ccccc45)c3)c3cccc4oc5ccccc5c34)cccc21]

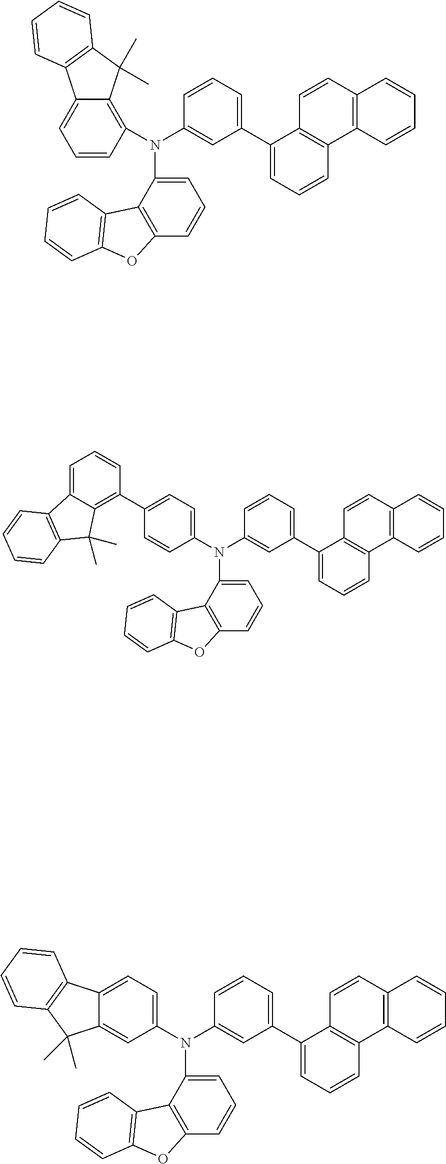 [SMILES: CC1(C)c2ccccc2-c2cccc(N(c3cccc(-c4cccc5c4ccc4ccccc45)c3)c3cccc4oc5ccccc5c34)c21 ; CC1(C)c2ccccc2-c2cccc(-c3ccc(N(c4cccc(-c5cccc6c5ccc5ccccc56)c4)c4cccc5oc6ccccc6c45)cc3)c21 ; CC1(C)c2ccccc2-c2ccc(N(c3cccc(-c4cccc5c4ccc4ccccc45)c3)c3cccc4oc5ccccc5c34)cc21]

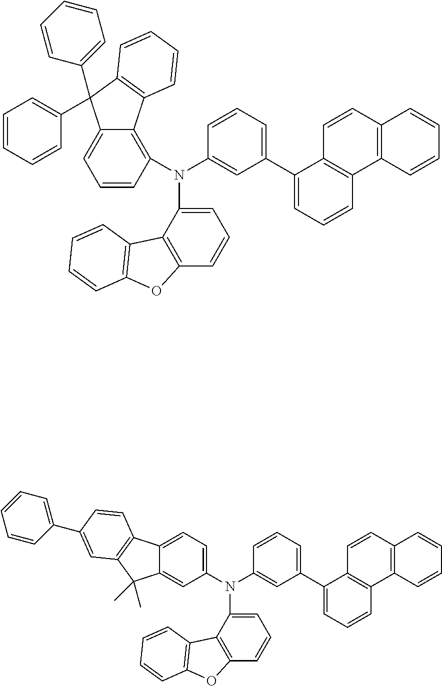 [SMILES: c1ccc(C2(c3ccccc3)c3ccccc3-c3c(N(c4cccc(-c5cccc6c5ccc5ccccc56)c4)c4cccc5oc6ccccc6c45)cccc32)cc1 ; CC1(C)c2cc(-c3ccccc3)ccc2-c2ccc(N(c3cccc(-c4cccc5c4ccc4ccccc45)c3)c3cccc4oc5ccccc5c34)cc21]

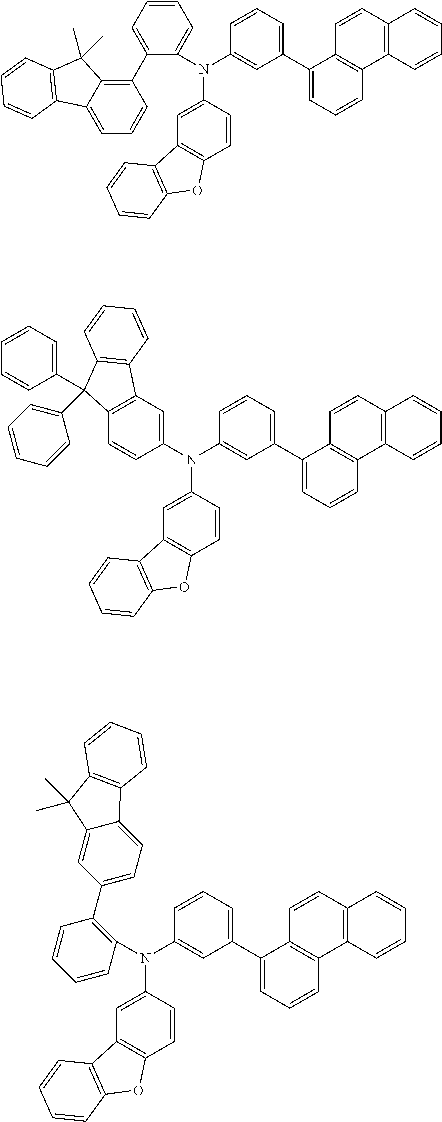 [SMILES: CC1(C)c2ccccc2-c2cccc(-c3ccccc3N(c3cccc(-c4cccc5c4ccc4ccccc45)c3)c3ccc4oc5ccccc5c4c3)c21 ; c1ccc(C2(c3ccccc3)c3ccccc3-c3cc(N(c4cccc(-c5cccc6c5ccc5ccccc56)c4)c4ccc5oc6ccccc6c5c4)ccc32)cc1 ; CC1(C)c2ccccc2-c2ccc(-c3ccccc3N(c3cccc(-c4cccc5c4ccc4ccccc45)c3)c3ccc4oc5ccccc5c4c3)cc21]

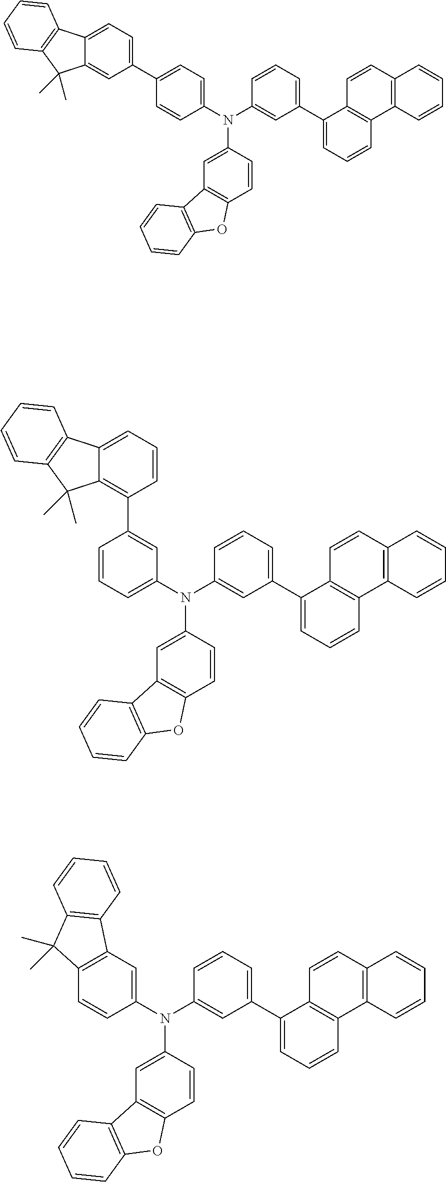 [SMILES: CC1(C)c2ccccc2-c2ccc(-c3ccc(N(c4cccc(-c5cccc6c5ccc5ccccc56)c4)c4ccc5oc6ccccc6c5c4)cc3)cc21 ; CC1(C)c2ccccc2-c2cccc(-c3cccc(N(c4cccc(-c5cccc6c5ccc5ccccc56)c4)c4ccc5oc6ccccc6c5c4)c3)c21 ; CC1(C)c2ccccc2-c2cc(N(c3cccc(-c4cccc5c4ccc4ccccc45)c3)c3ccc4oc5ccccc5c4c3)ccc21]

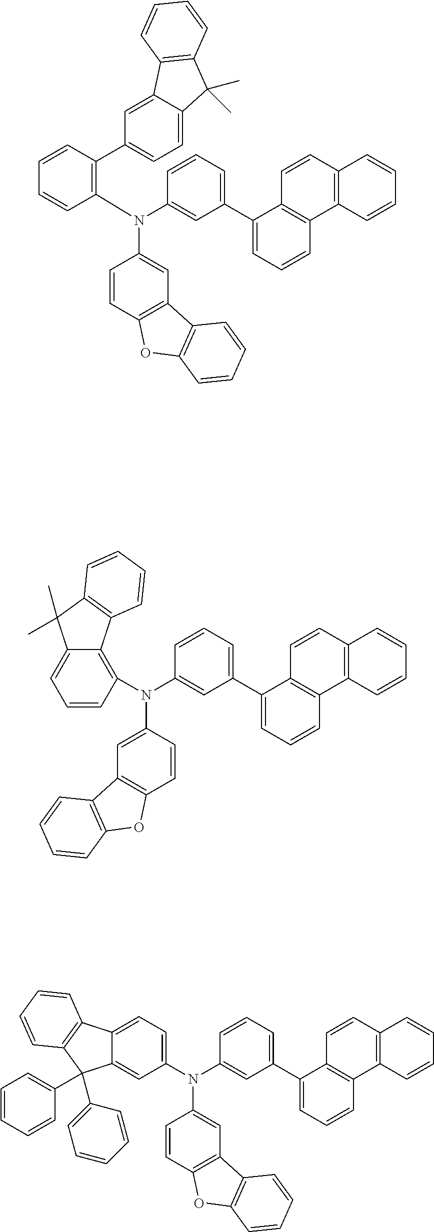 [SMILES: CC1(C)c2ccccc2-c2cc(-c3ccccc3N(c3cccc(-c4cccc5c4ccc4ccccc45)c3)c3ccc4oc5ccccc5c4c3)ccc21 ; CC1(C)c2ccccc2-c2c(N(c3cccc(-c4cccc5c4ccc4ccccc45)c3)c3ccc4oc5ccccc5c4c3)cccc21 ; c1ccc(C2(c3ccccc3)c3ccccc3-c3ccc(N(c4cccc(-c5cccc6c5ccc5ccccc56)c4)c4ccc5oc6ccccc6c5c4)cc32)cc1]

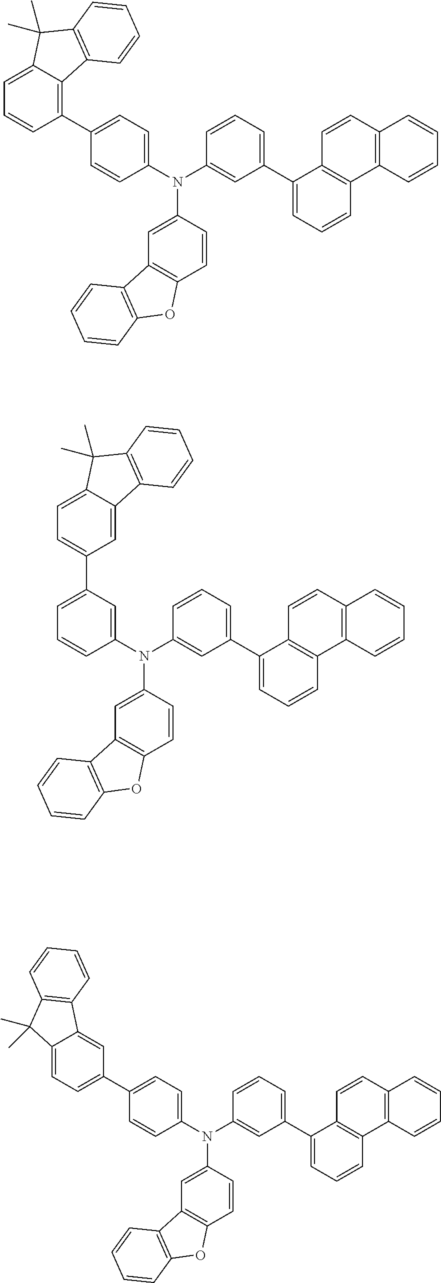 [SMILES: CC1(C)c2ccccc2-c2c(-c3ccc(N(c4cccc(-c5cccc6c5ccc5ccccc56)c4)c4ccc5oc6ccccc6c5c4)cc3)cccc21 ; CC1(C)c2ccccc2-c2cc(-c3cccc(N(c4cccc(-c5cccc6c5ccc5ccccc56)c4)c4ccc5oc6ccccc6c5c4)c3)ccc21 ; CC1(C)c2ccccc2-c2cc(-c3ccc(N(c4cccc(-c5cccc6c5ccc5ccccc56)c4)c4ccc5oc6ccccc6c5c4)cc3)ccc21]

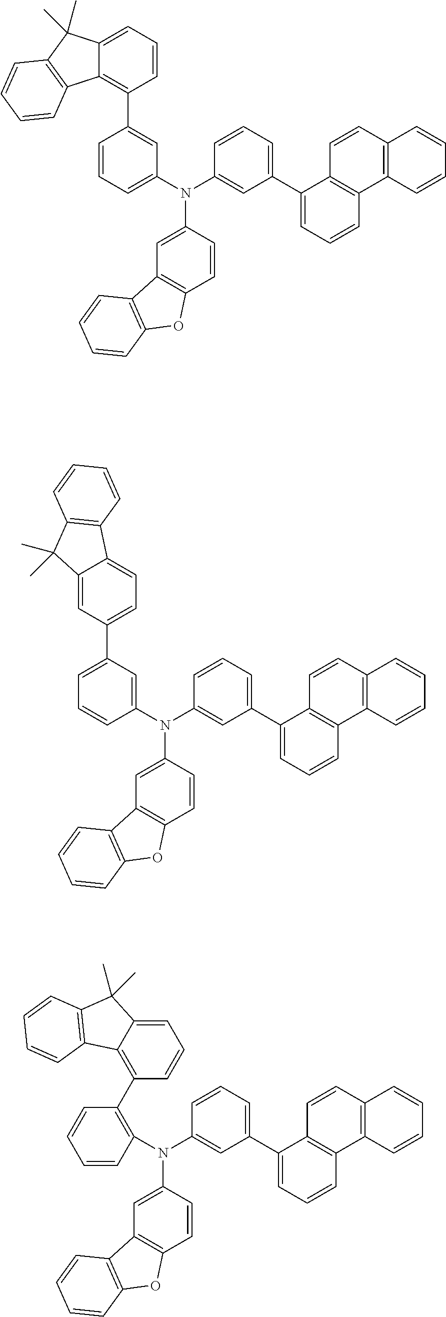 [SMILES: CC1(C)c2ccccc2-c2c(-c3cccc(N(c4cccc(-c5cccc6c5ccc5ccccc56)c4)c4ccc5oc6ccccc6c5c4)c3)cccc21 ; CC1(C)c2ccccc2-c2ccc(-c3cccc(N(c4cccc(-c5cccc6c5ccc5ccccc56)c4)c4ccc5oc6ccccc6c5c4)c3)cc21 ; CC1(C)c2ccccc2-c2c(-c3ccccc3N(c3cccc(-c4cccc5c4ccc4ccccc45)c3)c3ccc4oc5ccccc5c4c3)cccc21]

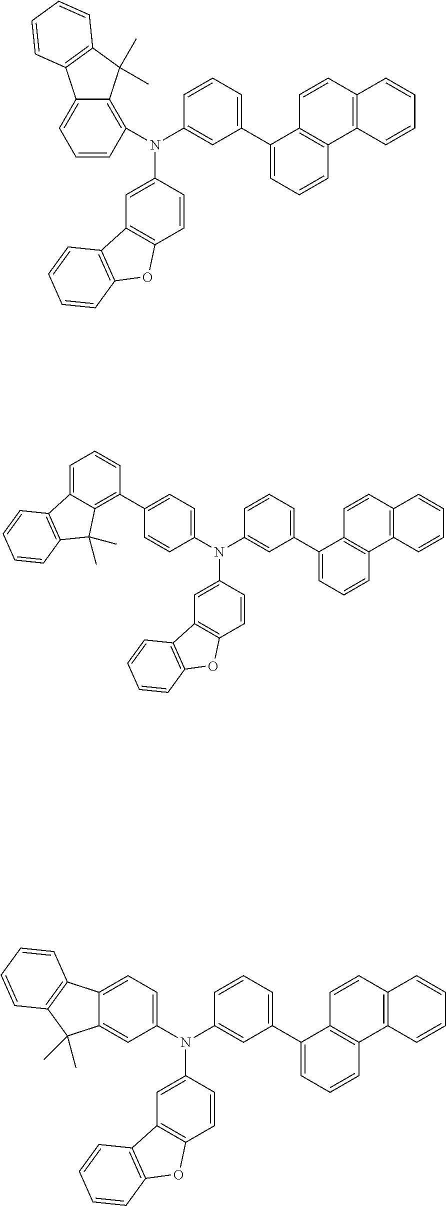 [SMILES: CC1(C)c2ccccc2-c2cccc(N(c3cccc(-c4cccc5c4ccc4ccccc45)c3)c3ccc4oc5ccccc5c4c3)c21 ; CC1(C)c2ccccc2-c2cccc(-c3ccc(N(c4cccc(-c5cccc6c5ccc5ccccc56)c4)c4ccc5oc6ccccc6c5c4)cc3)c21 ; CC1(C)c2ccccc2-c2ccc(N(c3cccc(-c4cccc5c4ccc4ccccc45)c3)c3ccc4oc5ccccc5c4c3)cc21]

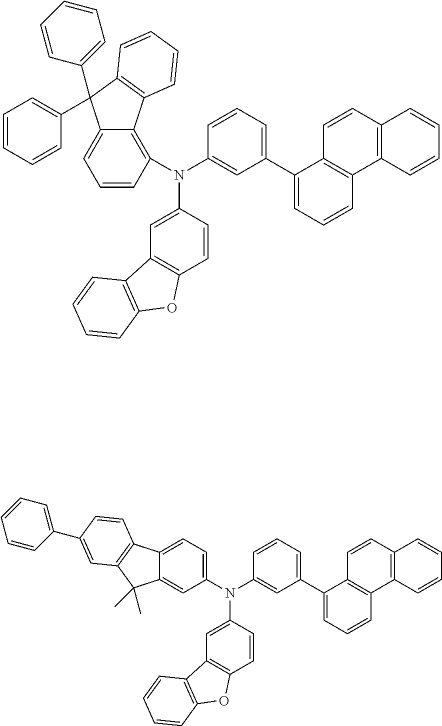 [SMILES: c1ccc(C2(c3ccccc3)c3ccccc3-c3c(N(c4cccc(-c5cccc6c5ccc5ccccc56)c4)c4ccc5oc6ccccc6c5c4)cccc32)cc1 ; CC1(C)c2cc(-c3ccccc3)ccc2-c2ccc(N(c3cccc(-c4cccc5c4ccc4ccccc45)c3)c3ccc4oc5ccccc5c4c3)cc21]

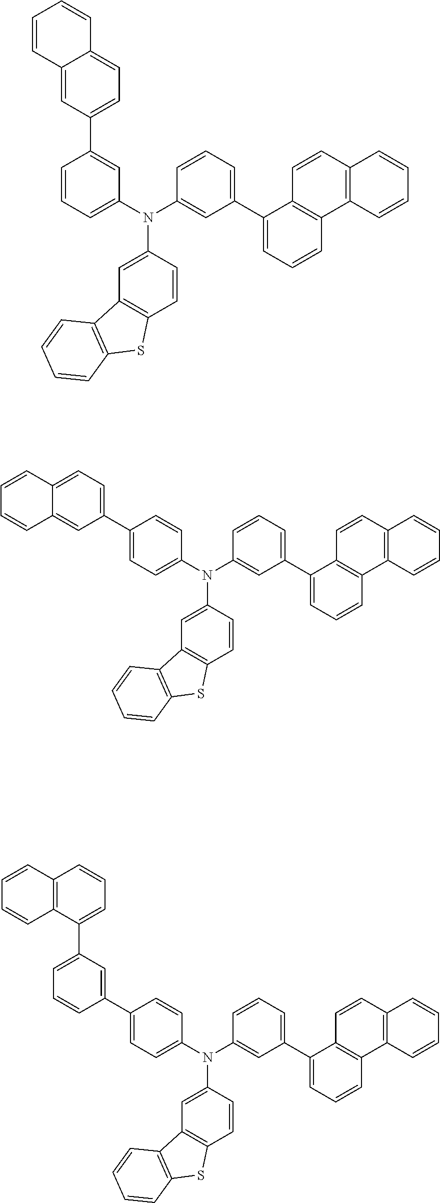 [SMILES: c1cc(-c2ccc3ccccc3c2)cc(N(c2cccc(-c3cccc4c3ccc3ccccc34)c2)c2ccc3sc4ccccc4c3c2)c1 ; c1cc(-c2cccc3c2ccc2ccccc23)cc(N(c2ccc(-c3ccc4ccccc4c3)cc2)c2ccc3sc4ccccc4c3c2)c1 ; c1cc(-c2ccc(N(c3cccc(-c4cccc5c4ccc4ccccc45)c3)c3ccc4sc5ccccc5c4c3)cc2)cc(-c2cccc3ccccc23)c1]

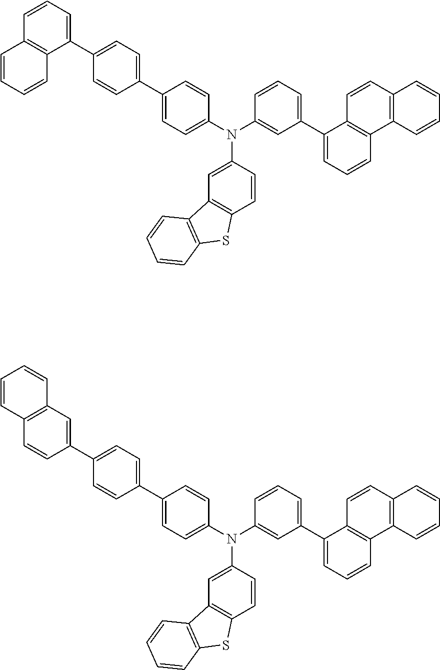 [SMILES: c1cc(-c2cccc3c2ccc2ccccc23)cc(N(c2ccc(-c3ccc(-c4cccc5ccccc45)cc3)cc2)c2ccc3sc4ccccc4c3c2)c1 ; c1cc(-c2cccc3c2ccc2ccccc23)cc(N(c2ccc(-c3ccc(-c4ccc5ccccc5c4)cc3)cc2)c2ccc3sc4ccccc4c3c2)c1]

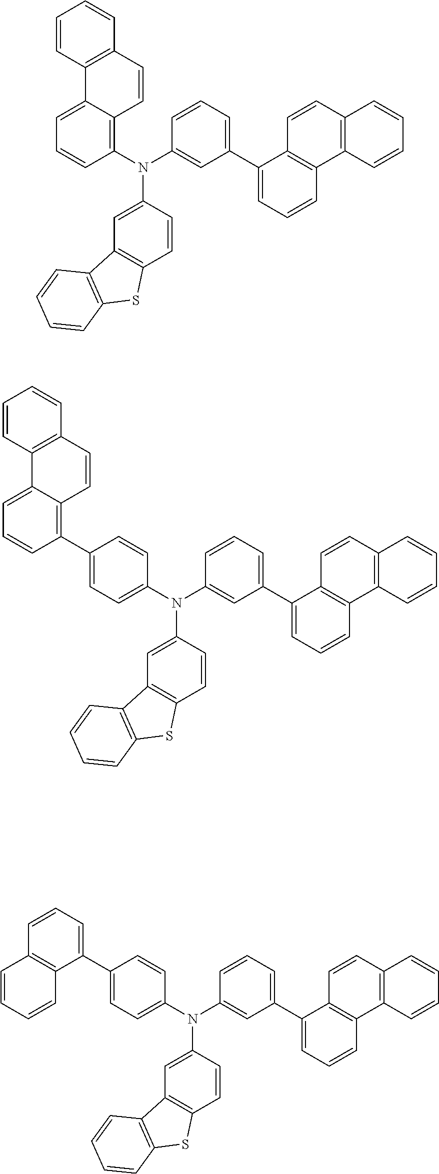 [SMILES: c1cc(-c2cccc3c2ccc2ccccc23)cc(N(c2ccc3sc4ccccc4c3c2)c2cccc3c2ccc2ccccc23)c1 ; c1cc(-c2cccc3c2ccc2ccccc23)cc(N(c2ccc(-c3cccc4c3ccc3ccccc34)cc2)c2ccc3sc4ccccc4c3c2)c1 ; c1cc(-c2cccc3c2ccc2ccccc23)cc(N(c2ccc(-c3cccc4ccccc34)cc2)c2ccc3sc4ccccc4c3c2)c1]

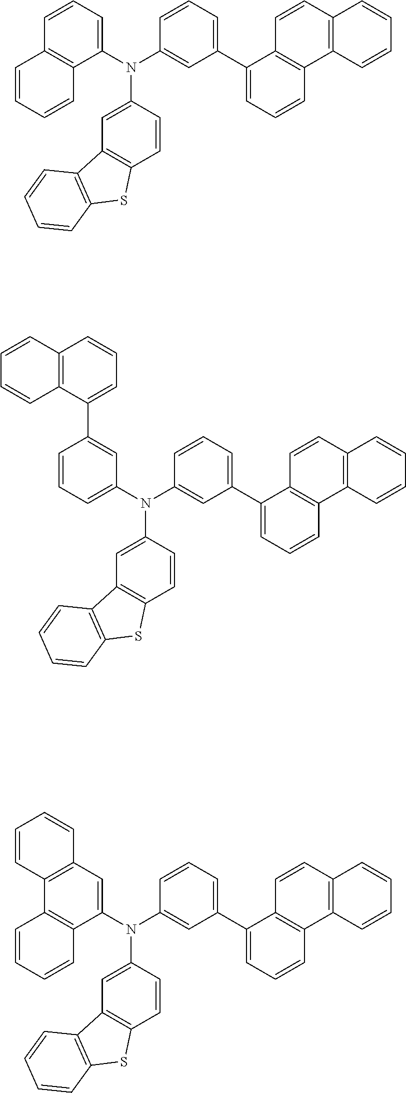 [SMILES: c1cc(-c2cccc3c2ccc2ccccc23)cc(N(c2ccc3sc4ccccc4c3c2)c2cccc3ccccc23)c1 ; c1cc(-c2cccc3ccccc23)cc(N(c2cccc(-c3cccc4c3ccc3ccccc34)c2)c2ccc3sc4ccccc4c3c2)c1 ; c1cc(-c2cccc3c2ccc2ccccc23)cc(N(c2ccc3sc4ccccc4c3c2)c2cc3ccccc3c3ccccc23)c1]

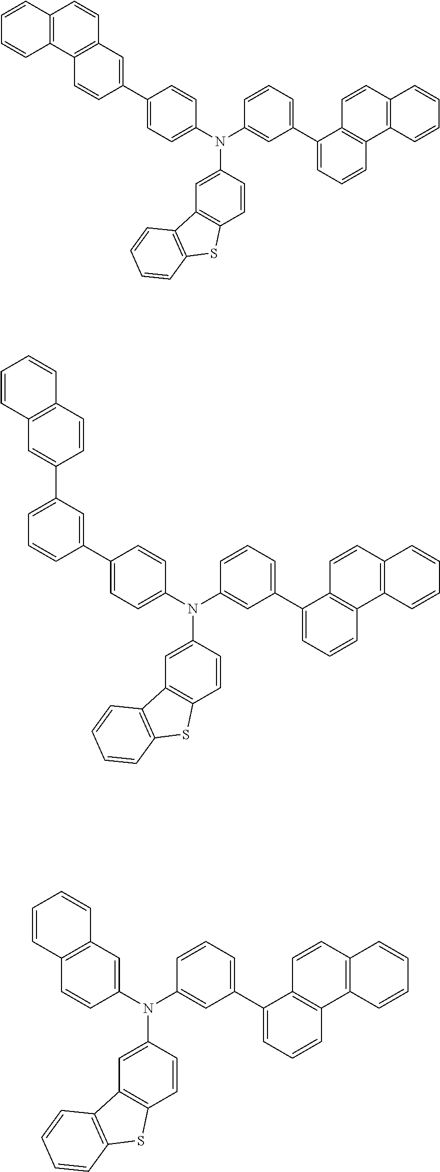 [SMILES: c1cc(-c2cccc3c2ccc2ccccc23)cc(N(c2ccc(-c3ccc4c(ccc5ccccc54)c3)cc2)c2ccc3sc4ccccc4c3c2)c1 ; c1cc(-c2ccc(N(c3cccc(-c4cccc5c4ccc4ccccc45)c3)c3ccc4sc5ccccc5c4c3)cc2)cc(-c2ccc3ccccc3c2)c1 ; c1cc(-c2cccc3c2ccc2ccccc23)cc(N(c2ccc3ccccc3c2)c2ccc3sc4ccccc4c3c2)c1]

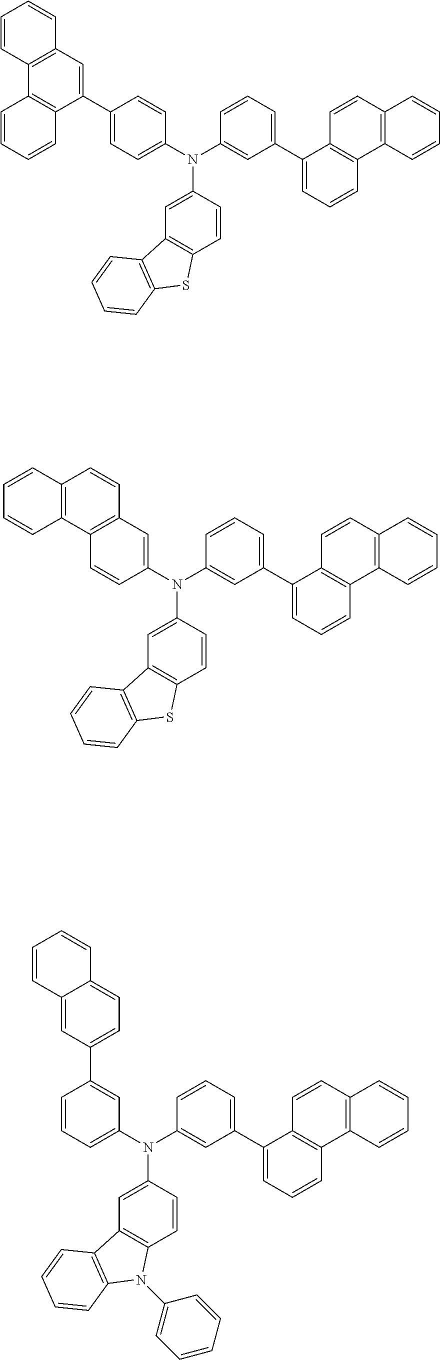 [SMILES: c1cc(-c2cccc3c2ccc2ccccc23)cc(N(c2ccc(-c3cc4ccccc4c4ccccc34)cc2)c2ccc3sc4ccccc4c3c2)c1 ; c1cc(-c2cccc3c2ccc2ccccc23)cc(N(c2ccc3c(ccc4ccccc43)c2)c2ccc3sc4ccccc4c3c2)c1 ; c1ccc(-n2c3ccccc3c3cc(N(c4cccc(-c5ccc6ccccc6c5)c4)c4cccc(-c5cccc6c5ccc5ccccc56)c4)ccc32)cc1]

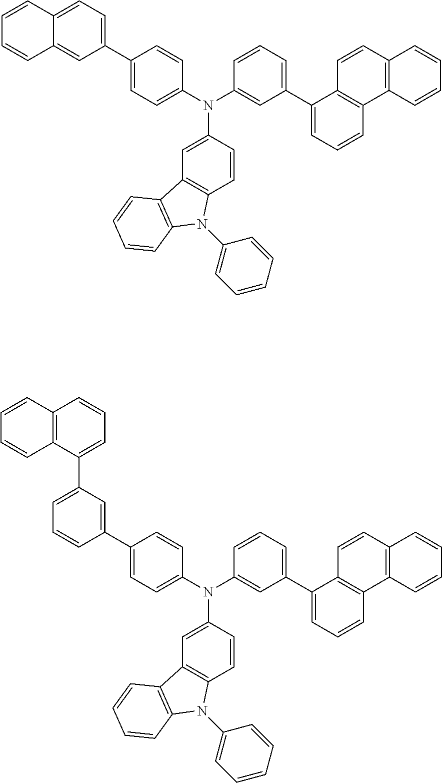 [SMILES: c1ccc(-n2c3ccccc3c3cc(N(c4ccc(-c5ccc6ccccc6c5)cc4)c4cccc(-c5cccc6c5ccc5ccccc56)c4)ccc32)cc1 ; c1ccc(-n2c3ccccc3c3cc(N(c4ccc(-c5cccc(-c6cccc7ccccc67)c5)cc4)c4cccc(-c5cccc6c5ccc5ccccc56)c4)ccc32)cc1]

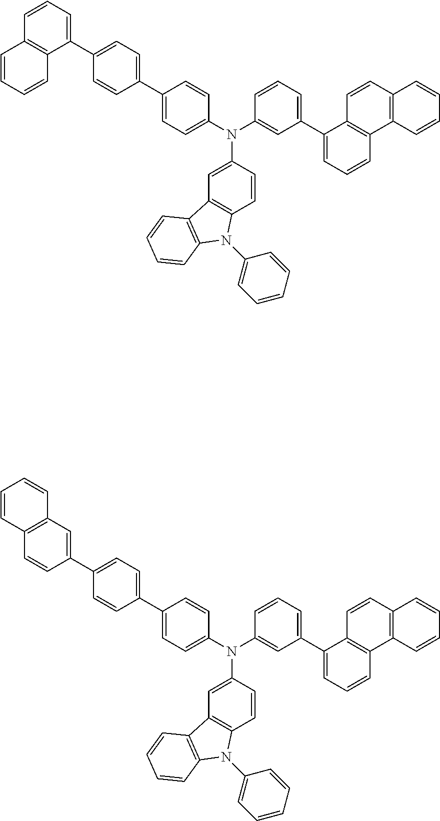 [SMILES: c1ccc(-n2c3ccccc3c3cc(N(c4ccc(-c5ccc(-c6cccc7ccccc67)cc5)cc4)c4cccc(-c5cccc6c5ccc5ccccc56)c4)ccc32)cc1 ; c1ccc(-n2c3ccccc3c3cc(N(c4ccc(-c5ccc(-c6ccc7ccccc7c6)cc5)cc4)c4cccc(-c5cccc6c5ccc5ccccc56)c4)ccc32)cc1]

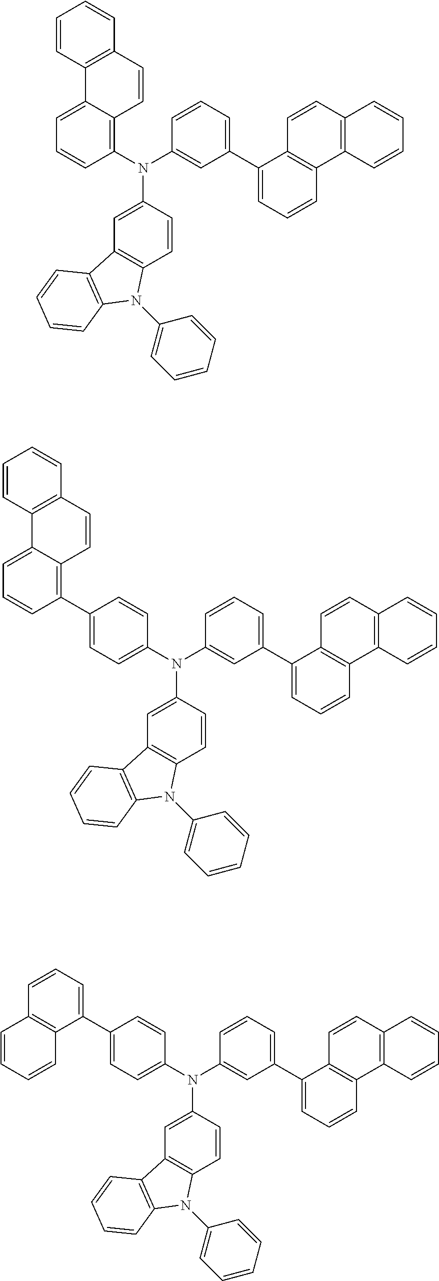 [SMILES: c1ccc(-n2c3ccccc3c3cc(N(c4cccc(-c5cccc6c5ccc5ccccc56)c4)c4cccc5c4ccc4ccccc45)ccc32)cc1 ; c1ccc(-n2c3ccccc3c3cc(N(c4ccc(-c5cccc6c5ccc5ccccc56)cc4)c4cccc(-c5cccc6c5ccc5ccccc56)c4)ccc32)cc1 ; c1ccc(-n2c3ccccc3c3cc(N(c4ccc(-c5cccc6ccccc56)cc4)c4cccc(-c5cccc6c5ccc5ccccc56)c4)ccc32)cc1]

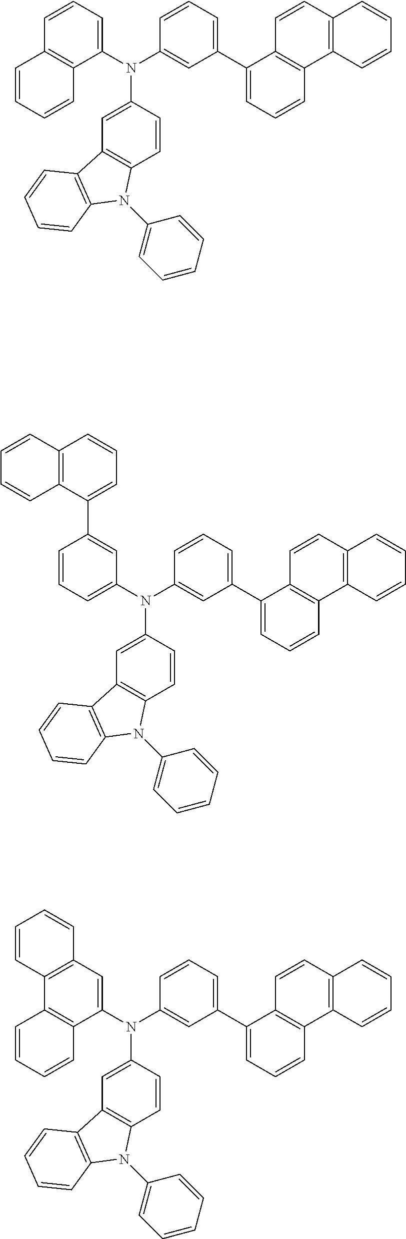 [SMILES: c1ccc(-n2c3ccccc3c3cc(N(c4cccc(-c5cccc6c5ccc5ccccc56)c4)c4cccc5ccccc45)ccc32)cc1 ; c1ccc(-n2c3ccccc3c3cc(N(c4cccc(-c5cccc6ccccc56)c4)c4cccc(-c5cccc6c5ccc5ccccc56)c4)ccc32)cc1 ; c1ccc(-n2c3ccccc3c3cc(N(c4cccc(-c5cccc6c5ccc5ccccc56)c4)c4cc5ccccc5c5ccccc45)ccc32)cc1]

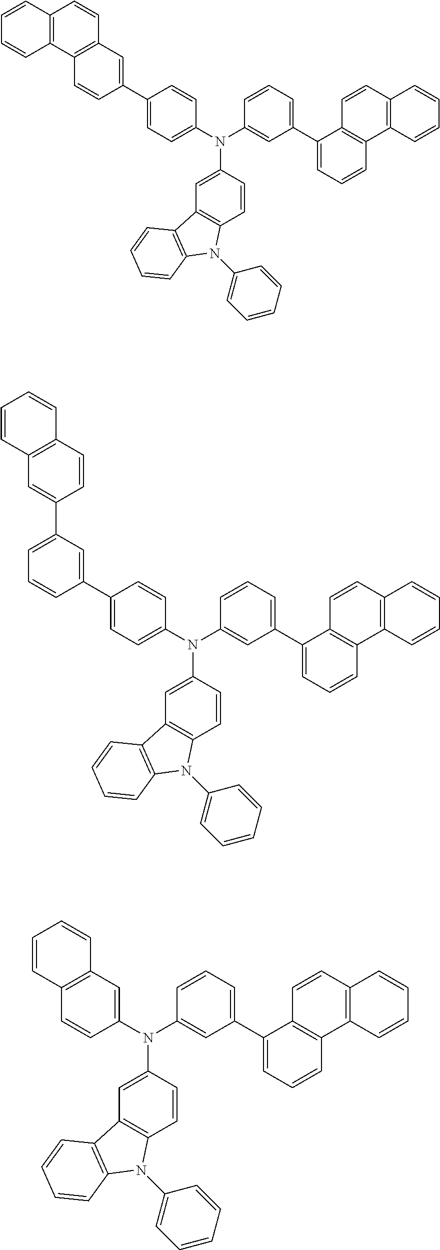 [SMILES: c1ccc(-n2c3ccccc3c3cc(N(c4ccc(-c5ccc6c(ccc7ccccc76)c5)cc4)c4cccc(-c5cccc6c5ccc5ccccc56)c4)ccc32)cc1 ; c1ccc(-n2c3ccccc3c3cc(N(c4ccc(-c5cccc(-c6ccc7ccccc7c6)c5)cc4)c4cccc(-c5cccc6c5ccc5ccccc56)c4)ccc32)cc1 ; c1ccc(-n2c3ccccc3c3cc(N(c4cccc(-c5cccc6c5ccc5ccccc56)c4)c4ccc5ccccc5c4)ccc32)cc1]

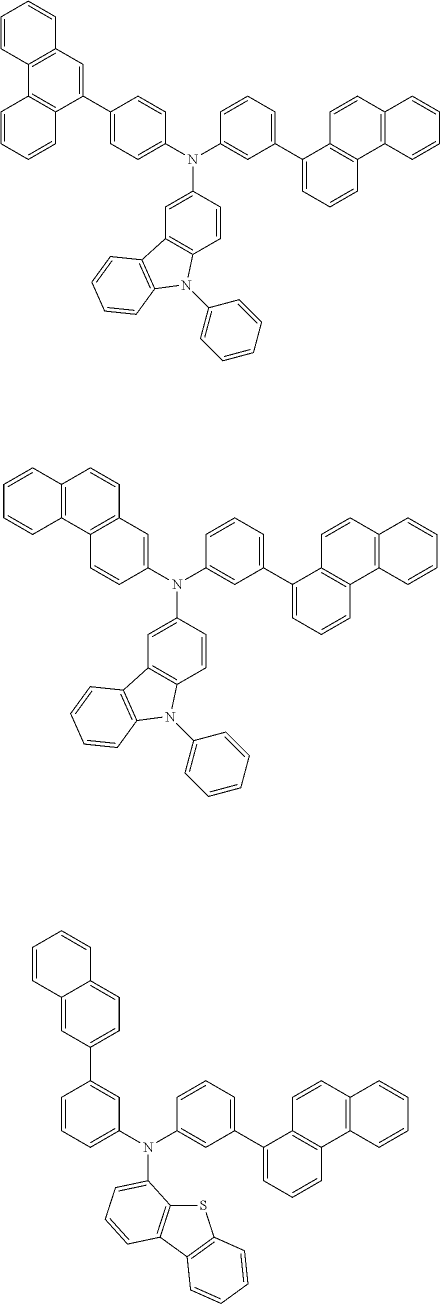 [SMILES: c1ccc(-n2c3ccccc3c3cc(N(c4ccc(-c5cc6ccccc6c6ccccc56)cc4)c4cccc(-c5cccc6c5ccc5ccccc56)c4)ccc32)cc1 ; c1ccc(-n2c3ccccc3c3cc(N(c4cccc(-c5cccc6c5ccc5ccccc56)c4)c4ccc5c(ccc6ccccc65)c4)ccc32)cc1 ; c1cc(-c2ccc3ccccc3c2)cc(N(c2cccc(-c3cccc4c3ccc3ccccc34)c2)c2cccc3c2sc2ccccc23)c1]

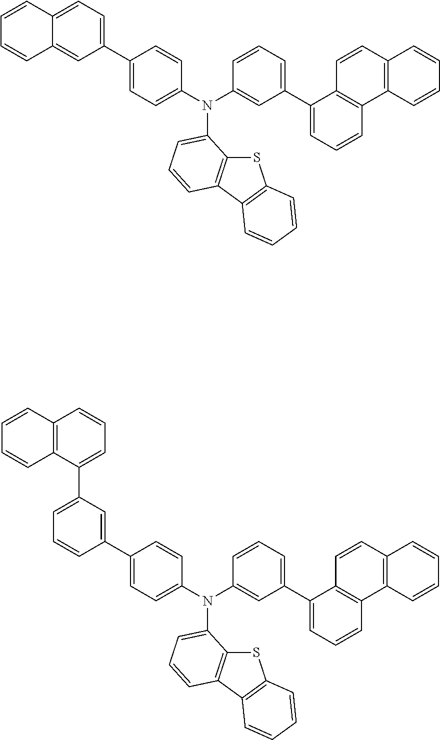 [SMILES: c1cc(-c2cccc3c2ccc2ccccc23)cc(N(c2ccc(-c3ccc4ccccc4c3)cc2)c2cccc3c2sc2ccccc23)c1 ; c1cc(-c2ccc(N(c3cccc(-c4cccc5c4ccc4ccccc45)c3)c3cccc4c3sc3ccccc34)cc2)cc(-c2cccc3ccccc23)c1]

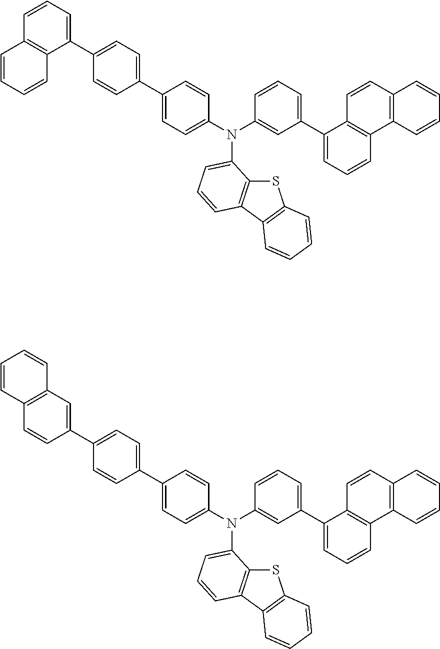 [SMILES: c1cc(-c2cccc3c2ccc2ccccc23)cc(N(c2ccc(-c3ccc(-c4cccc5ccccc45)cc3)cc2)c2cccc3c2sc2ccccc23)c1 ; c1cc(-c2cccc3c2ccc2ccccc23)cc(N(c2ccc(-c3ccc(-c4ccc5ccccc5c4)cc3)cc2)c2cccc3c2sc2ccccc23)c1]

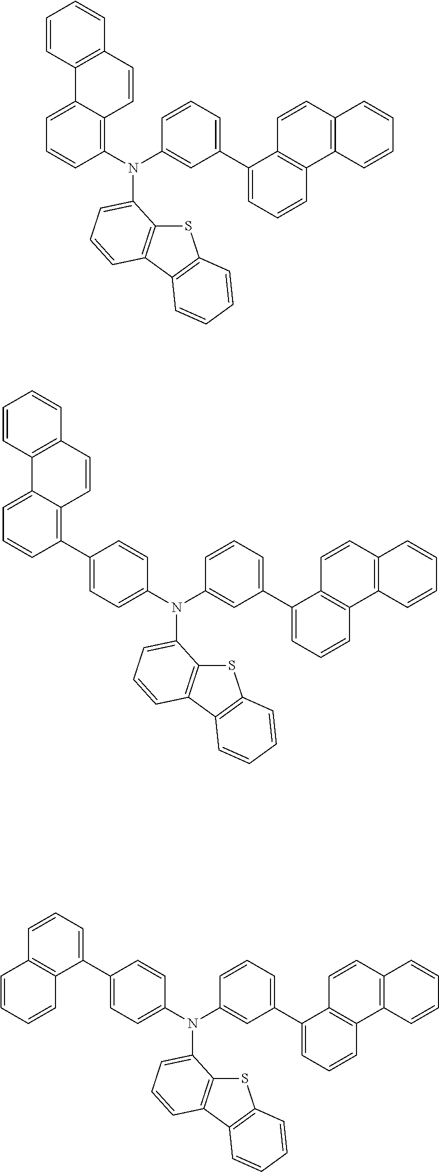 [SMILES: c1cc(-c2cccc3c2ccc2ccccc23)cc(N(c2cccc3c2ccc2ccccc23)c2cccc3c2sc2ccccc23)c1 ; c1cc(-c2cccc3c2ccc2ccccc23)cc(N(c2ccc(-c3cccc4c3ccc3ccccc34)cc2)c2cccc3c2sc2ccccc23)c1 ; c1cc(-c2cccc3c2ccc2ccccc23)cc(N(c2ccc(-c3cccc4ccccc34)cc2)c2cccc3c2sc2ccccc23)c1]

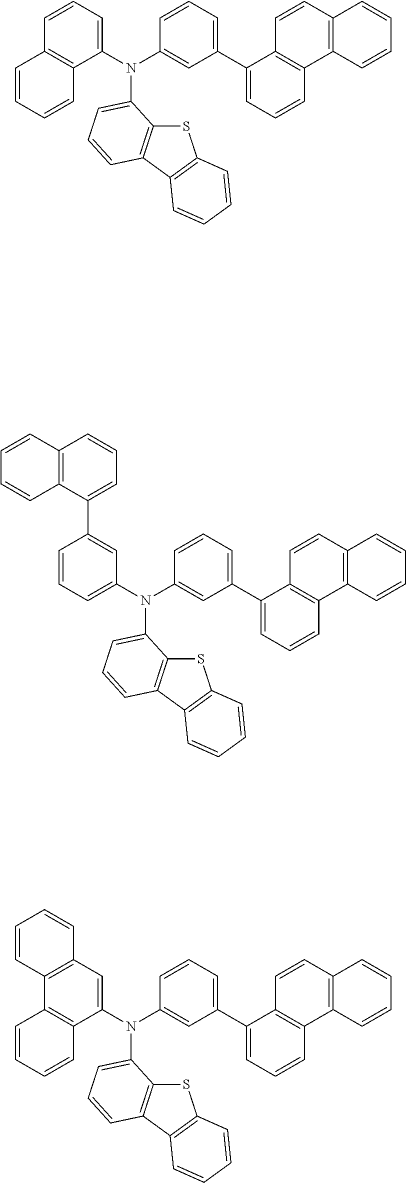 [SMILES: c1cc(-c2cccc3c2ccc2ccccc23)cc(N(c2cccc3ccccc23)c2cccc3c2sc2ccccc23)c1 ; c1cc(-c2cccc3ccccc23)cc(N(c2cccc(-c3cccc4c3ccc3ccccc34)c2)c2cccc3c2sc2ccccc23)c1 ; c1cc(-c2cccc3c2ccc2ccccc23)cc(N(c2cc3ccccc3c3ccccc23)c2cccc3c2sc2ccccc23)c1]

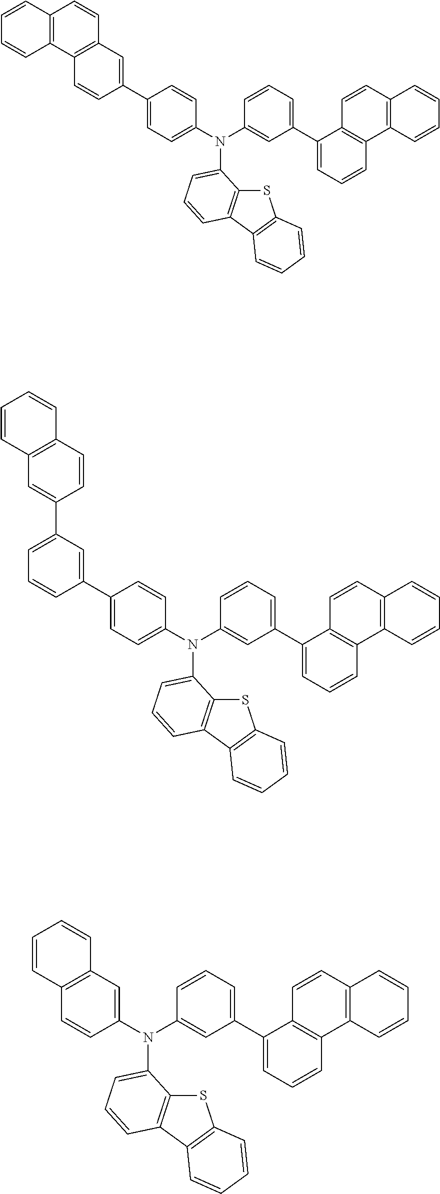 [SMILES: c1cc(-c2cccc3c2ccc2ccccc23)cc(N(c2ccc(-c3ccc4c(ccc5ccccc54)c3)cc2)c2cccc3c2sc2ccccc23)c1 ; c1cc(-c2ccc(N(c3cccc(-c4cccc5c4ccc4ccccc45)c3)c3cccc4c3sc3ccccc34)cc2)cc(-c2ccc3ccccc3c2)c1 ; c1cc(-c2cccc3c2ccc2ccccc23)cc(N(c2ccc3ccccc3c2)c2cccc3c2sc2ccccc23)c1]

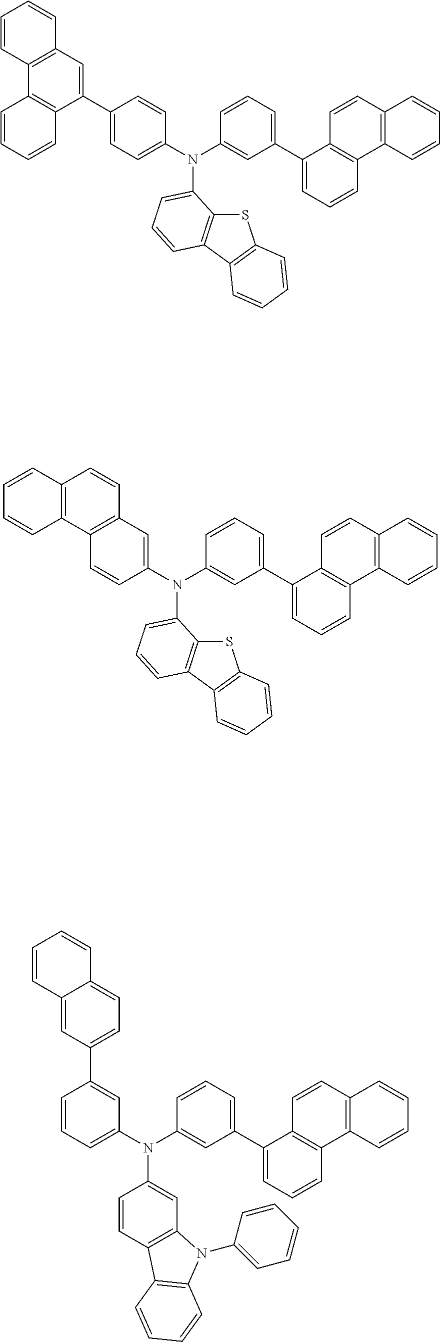 [SMILES: c1cc(-c2cccc3c2ccc2ccccc23)cc(N(c2ccc(-c3cc4ccccc4c4ccccc34)cc2)c2cccc3c2sc2ccccc23)c1 ; c1cc(-c2cccc3c2ccc2ccccc23)cc(N(c2ccc3c(ccc4ccccc43)c2)c2cccc3c2sc2ccccc23)c1 ; c1ccc(-n2c3ccccc3c3ccc(N(c4cccc(-c5ccc6ccccc6c5)c4)c4cccc(-c5cccc6c5ccc5ccccc56)c4)cc32)cc1]

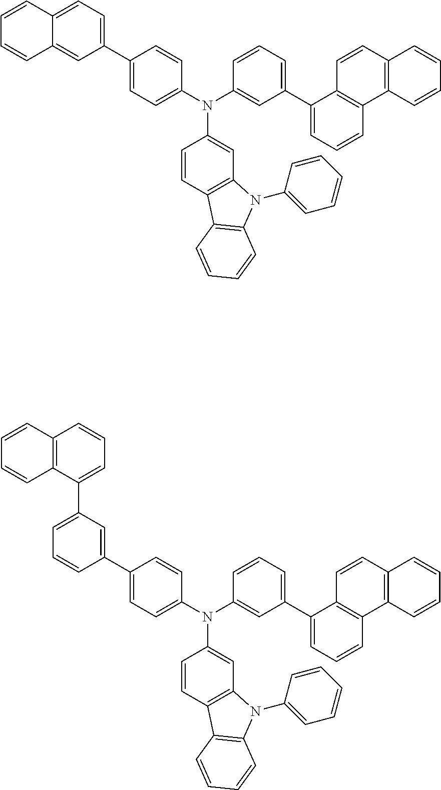 [SMILES: c1ccc(-n2c3ccccc3c3ccc(N(c4ccc(-c5ccc6ccccc6c5)cc4)c4cccc(-c5cccc6c5ccc5ccccc56)c4)cc32)cc1 ; c1ccc(-n2c3ccccc3c3ccc(N(c4ccc(-c5cccc(-c6cccc7ccccc67)c5)cc4)c4cccc(-c5cccc6c5ccc5ccccc56)c4)cc32)cc1]

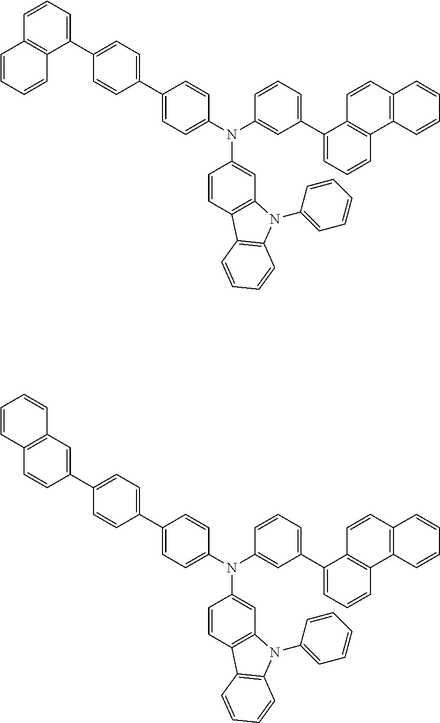 [SMILES: c1ccc(-n2c3ccccc3c3ccc(N(c4ccc(-c5ccc(-c6cccc7ccccc67)cc5)cc4)c4cccc(-c5cccc6c5ccc5ccccc56)c4)cc32)cc1 ; c1ccc(-n2c3ccccc3c3ccc(N(c4ccc(-c5ccc(-c6ccc7ccccc7c6)cc5)cc4)c4cccc(-c5cccc6c5ccc5ccccc56)c4)cc32)cc1]

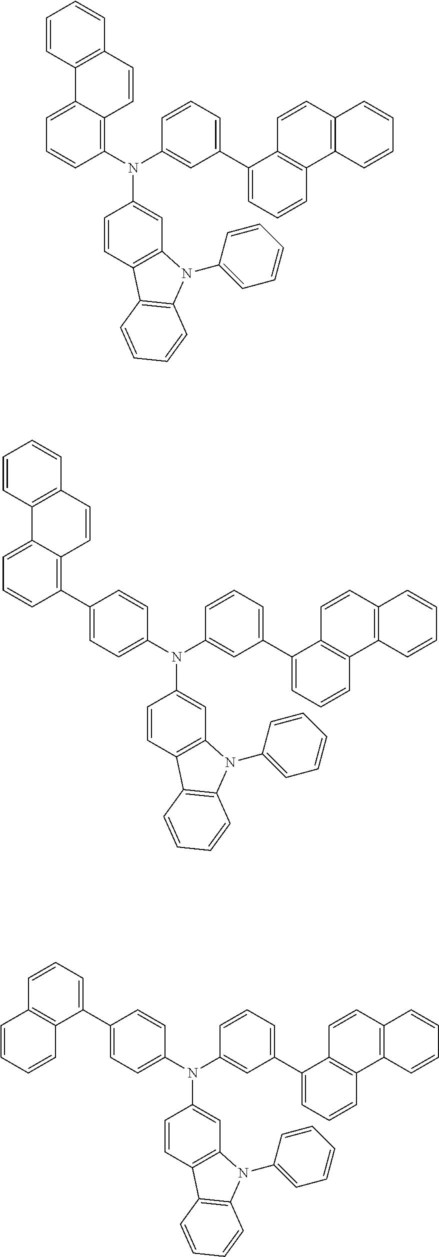 [SMILES: c1ccc(-n2c3ccccc3c3ccc(N(c4cccc(-c5cccc6c5ccc5ccccc56)c4)c4cccc5c4ccc4ccccc45)cc32)cc1 ; c1ccc(-n2c3ccccc3c3ccc(N(c4ccc(-c5cccc6c5ccc5ccccc56)cc4)c4cccc(-c5cccc6c5ccc5ccccc56)c4)cc32)cc1 ; c1ccc(-n2c3ccccc3c3ccc(N(c4ccc(-c5cccc6ccccc56)cc4)c4cccc(-c5cccc6c5ccc5ccccc56)c4)cc32)cc1]

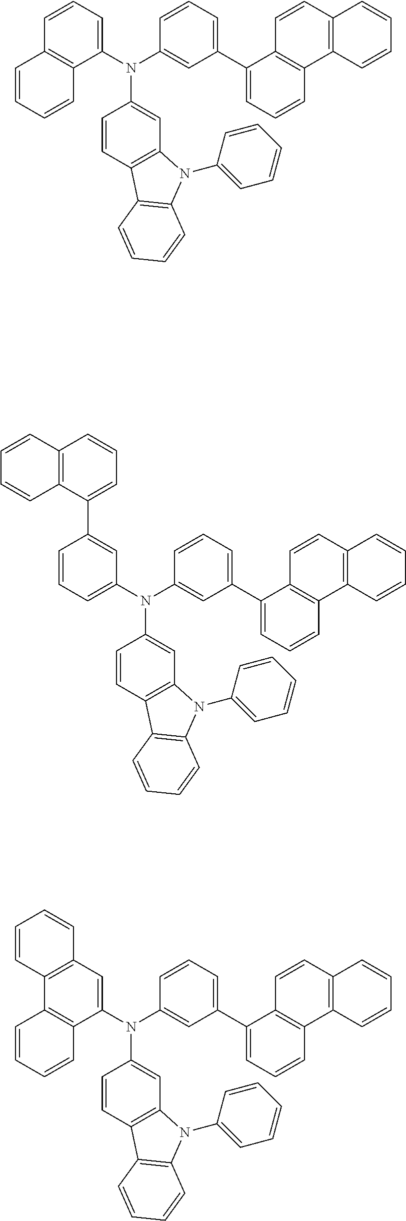 [SMILES: c1ccc(-n2c3ccccc3c3ccc(N(c4cccc(-c5cccc6c5ccc5ccccc56)c4)c4cccc5ccccc45)cc32)cc1 ; c1ccc(-n2c3ccccc3c3ccc(N(c4cccc(-c5cccc6ccccc56)c4)c4cccc(-c5cccc6c5ccc5ccccc56)c4)cc32)cc1 ; c1ccc(-n2c3ccccc3c3ccc(N(c4cccc(-c5cccc6c5ccc5ccccc56)c4)c4cc5ccccc5c5ccccc45)cc32)cc1]

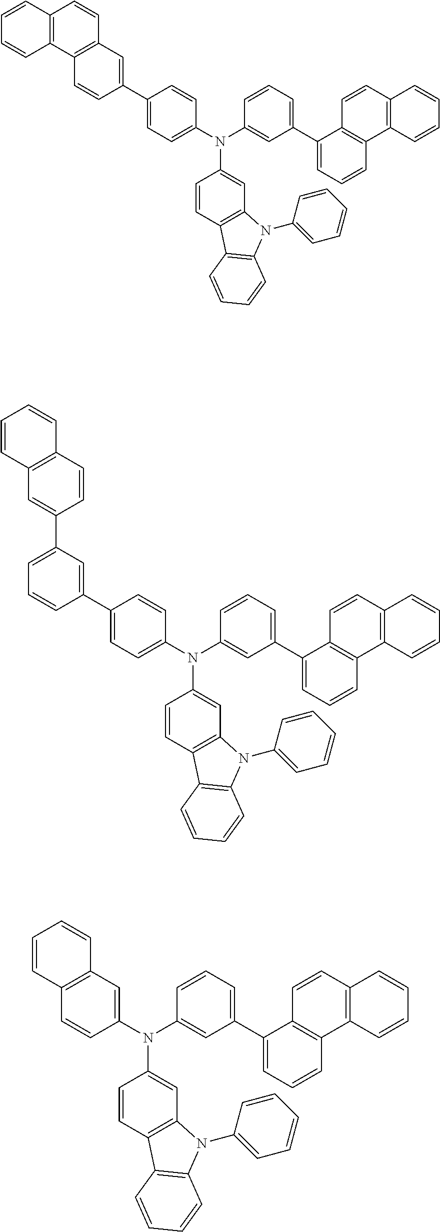 [SMILES: c1ccc(-n2c3ccccc3c3ccc(N(c4ccc(-c5ccc6c(ccc7ccccc76)c5)cc4)c4cccc(-c5cccc6c5ccc5ccccc56)c4)cc32)cc1 ; c1ccc(-n2c3ccccc3c3ccc(N(c4ccc(-c5cccc(-c6ccc7ccccc7c6)c5)cc4)c4cccc(-c5cccc6c5ccc5ccccc56)c4)cc32)cc1 ; c1ccc(-n2c3ccccc3c3ccc(N(c4cccc(-c5cccc6c5ccc5ccccc56)c4)c4ccc5ccccc5c4)cc32)cc1]

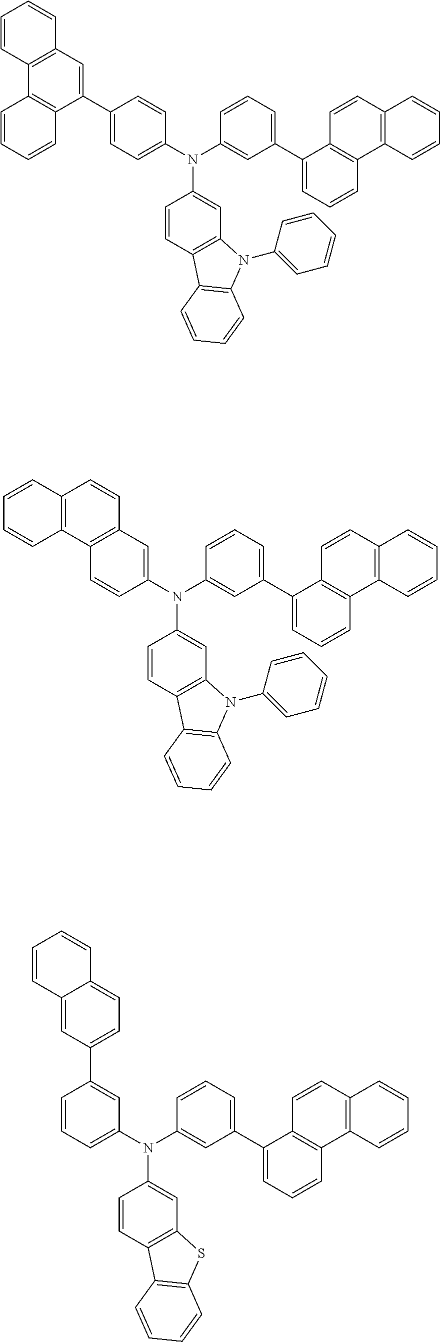 [SMILES: c1ccc(-n2c3ccccc3c3ccc(N(c4ccc(-c5cc6ccccc6c6ccccc56)cc4)c4cccc(-c5cccc6c5ccc5ccccc56)c4)cc32)cc1 ; c1ccc(-n2c3ccccc3c3ccc(N(c4cccc(-c5cccc6c5ccc5ccccc56)c4)c4ccc5c(ccc6ccccc65)c4)cc32)cc1 ; c1cc(-c2ccc3ccccc3c2)cc(N(c2cccc(-c3cccc4c3ccc3ccccc34)c2)c2ccc3c(c2)sc2ccccc23)c1]

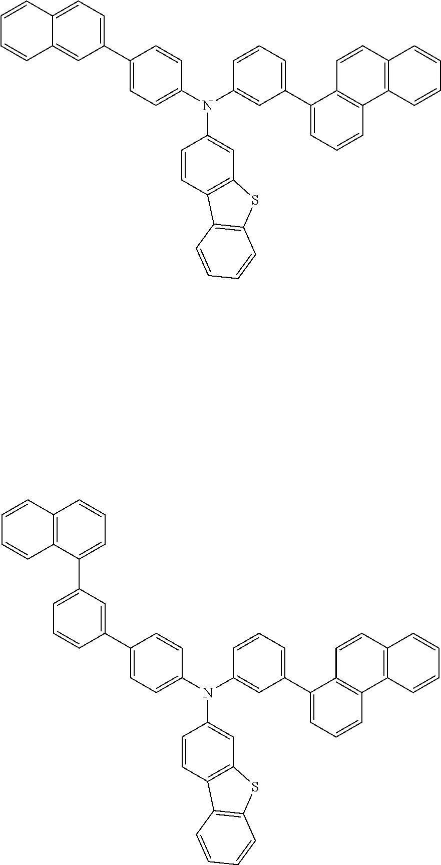 [SMILES: c1cc(-c2cccc3c2ccc2ccccc23)cc(N(c2ccc(-c3ccc4ccccc4c3)cc2)c2ccc3c(c2)sc2ccccc23)c1 ; c1cc(-c2ccc(N(c3cccc(-c4cccc5c4ccc4ccccc45)c3)c3ccc4c(c3)sc3ccccc34)cc2)cc(-c2cccc3ccccc23)c1]

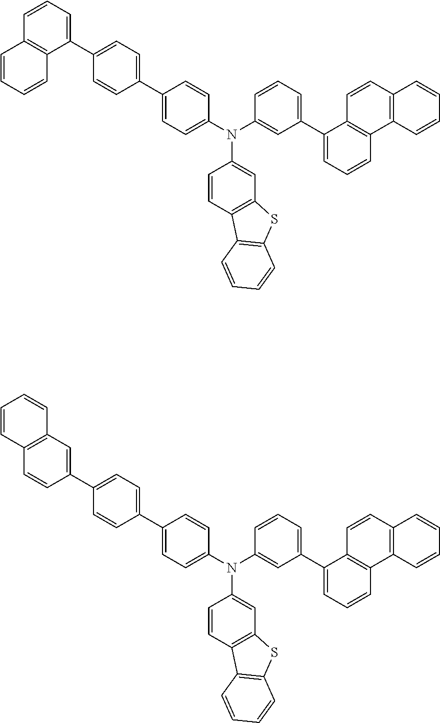 [SMILES: c1cc(-c2cccc3c2ccc2ccccc23)cc(N(c2ccc(-c3ccc(-c4cccc5ccccc45)cc3)cc2)c2ccc3c(c2)sc2ccccc23)c1 ; c1cc(-c2cccc3c2ccc2ccccc23)cc(N(c2ccc(-c3ccc(-c4ccc5ccccc5c4)cc3)cc2)c2ccc3c(c2)sc2ccccc23)c1]

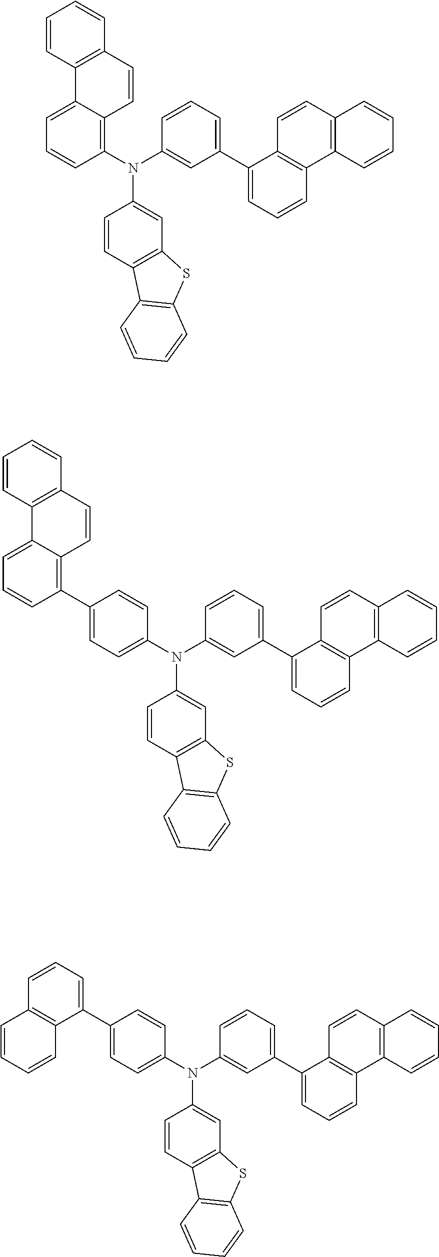 [SMILES: c1cc(-c2cccc3c2ccc2ccccc23)cc(N(c2ccc3c(c2)sc2ccccc23)c2cccc3c2ccc2ccccc23)c1 ; c1cc(-c2cccc3c2ccc2ccccc23)cc(N(c2ccc(-c3cccc4c3ccc3ccccc34)cc2)c2ccc3c(c2)sc2ccccc23)c1 ; c1cc(-c2cccc3c2ccc2ccccc23)cc(N(c2ccc(-c3cccc4ccccc34)cc2)c2ccc3c(c2)sc2ccccc23)c1]

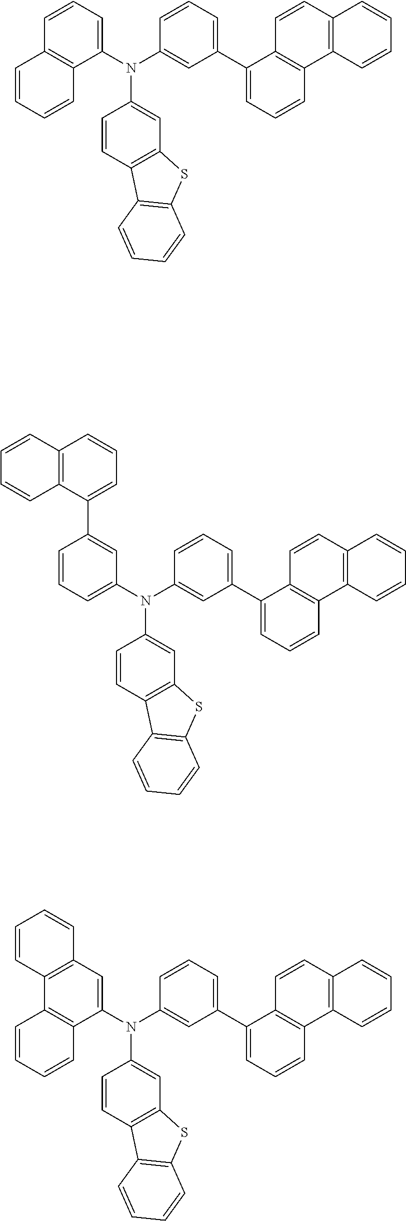 [SMILES: c1cc(-c2cccc3c2ccc2ccccc23)cc(N(c2ccc3c(c2)sc2ccccc23)c2cccc3ccccc23)c1 ; c1cc(-c2cccc3ccccc23)cc(N(c2cccc(-c3cccc4c3ccc3ccccc34)c2)c2ccc3c(c2)sc2ccccc23)c1 ; c1cc(-c2cccc3c2ccc2ccccc23)cc(N(c2ccc3c(c2)sc2ccccc23)c2cc3ccccc3c3ccccc23)c1]

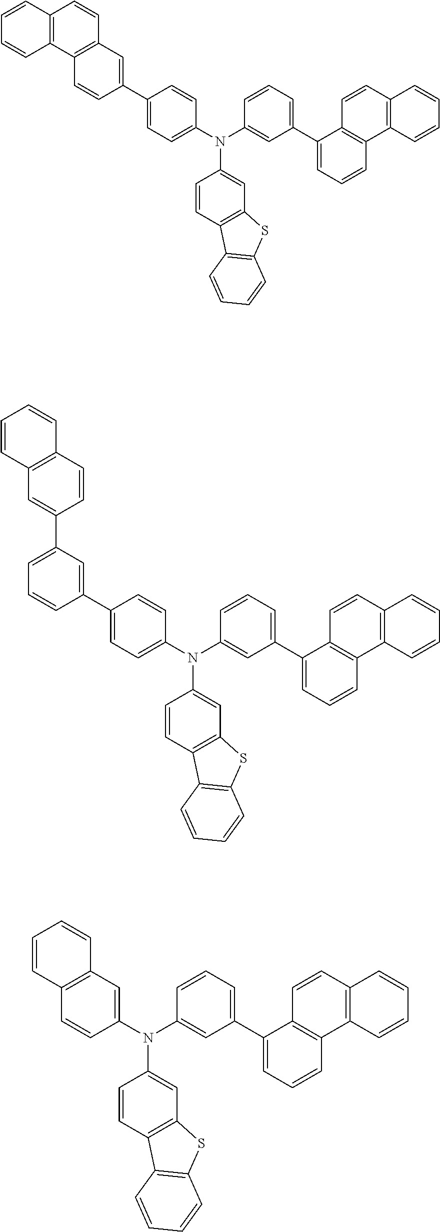 [SMILES: c1cc(-c2cccc3c2ccc2ccccc23)cc(N(c2ccc(-c3ccc4c(ccc5ccccc54)c3)cc2)c2ccc3c(c2)sc2ccccc23)c1 ; c1cc(-c2ccc(N(c3cccc(-c4cccc5c4ccc4ccccc45)c3)c3ccc4c(c3)sc3ccccc34)cc2)cc(-c2ccc3ccccc3c2)c1 ; c1cc(-c2cccc3c2ccc2ccccc23)cc(N(c2ccc3ccccc3c2)c2ccc3c(c2)sc2ccccc23)c1]

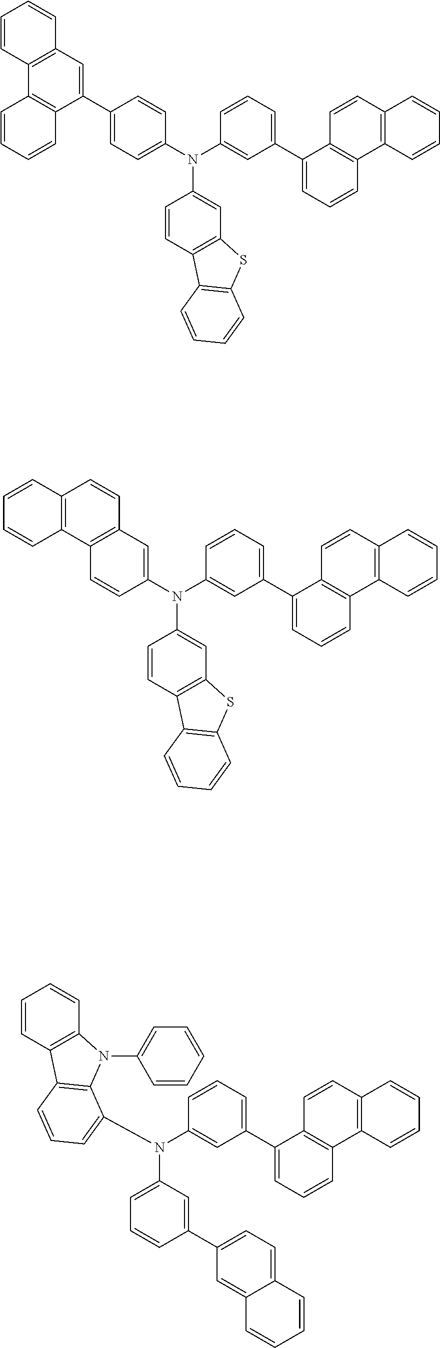 [SMILES: c1cc(-c2cccc3c2ccc2ccccc23)cc(N(c2ccc(-c3cc4ccccc4c4ccccc34)cc2)c2ccc3c(c2)sc2ccccc23)c1 ; c1cc(-c2cccc3c2ccc2ccccc23)cc(N(c2ccc3c(ccc4ccccc43)c2)c2ccc3c(c2)sc2ccccc23)c1 ; c1ccc(-n2c3ccccc3c3cccc(N(c4cccc(-c5ccc6ccccc6c5)c4)c4cccc(-c5cccc6c5ccc5ccccc56)c4)c32)cc1]

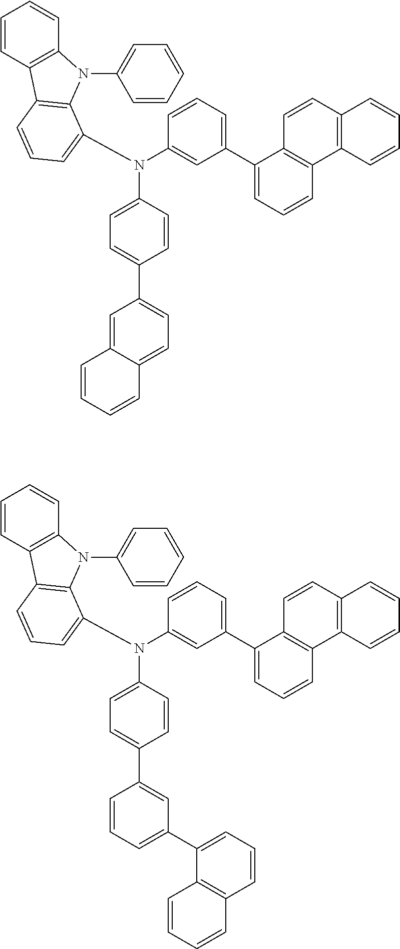 [SMILES: c1ccc(-n2c3ccccc3c3cccc(N(c4ccc(-c5ccc6ccccc6c5)cc4)c4cccc(-c5cccc6c5ccc5ccccc56)c4)c32)cc1 ; c1ccc(-n2c3ccccc3c3cccc(N(c4ccc(-c5cccc(-c6cccc7ccccc67)c5)cc4)c4cccc(-c5cccc6c5ccc5ccccc56)c4)c32)cc1]

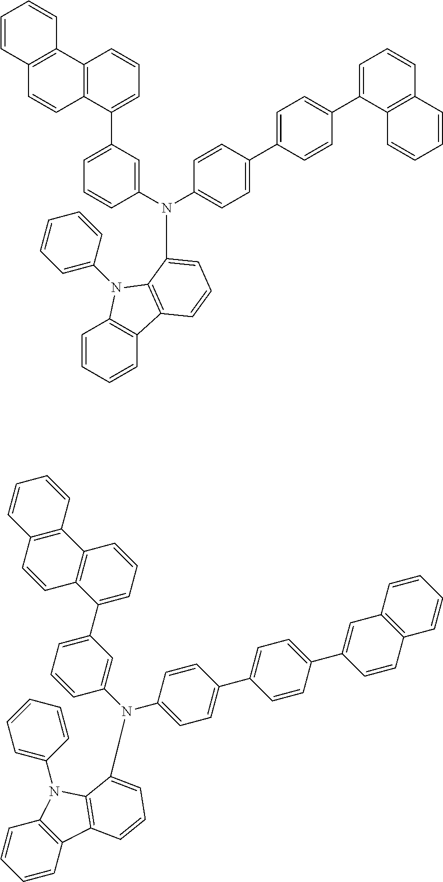 [SMILES: c1ccc(-n2c3ccccc3c3cccc(N(c4ccc(-c5ccc(-c6cccc7ccccc67)cc5)cc4)c4cccc(-c5cccc6c5ccc5ccccc56)c4)c32)cc1 ; c1ccc(-n2c3ccccc3c3cccc(N(c4ccc(-c5ccc(-c6ccc7ccccc7c6)cc5)cc4)c4cccc(-c5cccc6c5ccc5ccccc56)c4)c32)cc1]

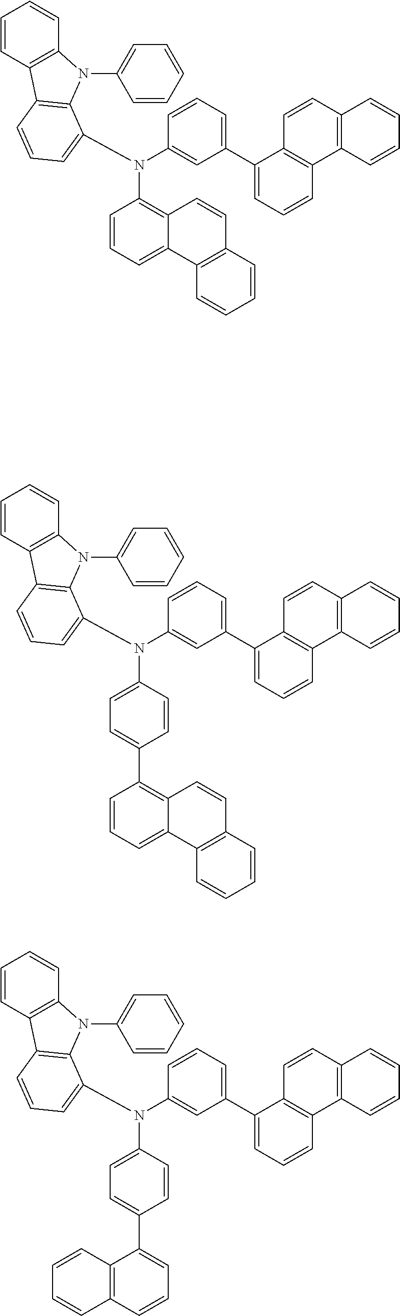 [SMILES: c1ccc(-n2c3ccccc3c3cccc(N(c4cccc(-c5cccc6c5ccc5ccccc56)c4)c4cccc5c4ccc4ccccc45)c32)cc1 ; c1ccc(-n2c3ccccc3c3cccc(N(c4ccc(-c5cccc6c5ccc5ccccc56)cc4)c4cccc(-c5cccc6c5ccc5ccccc56)c4)c32)cc1 ; c1ccc(-n2c3ccccc3c3cccc(N(c4ccc(-c5cccc6ccccc56)cc4)c4cccc(-c5cccc6c5ccc5ccccc56)c4)c32)cc1]

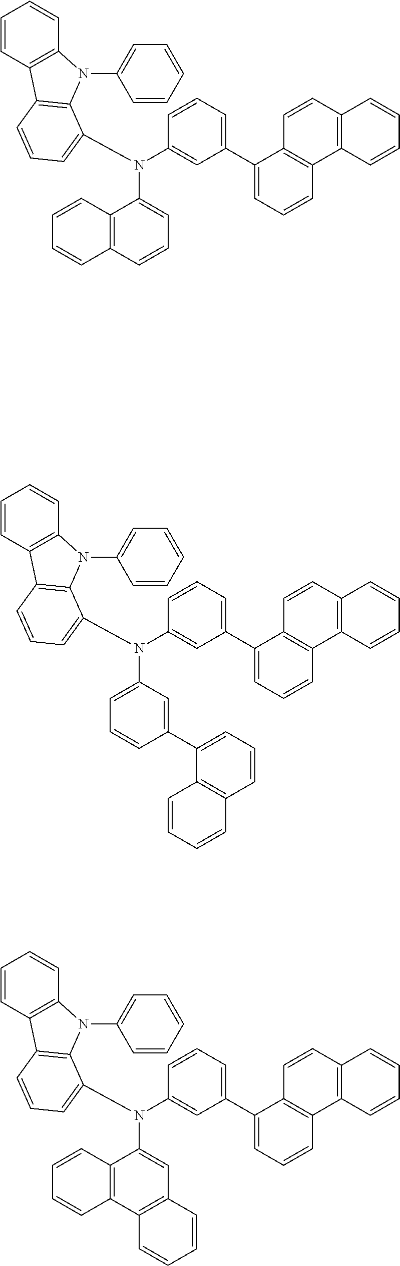 [SMILES: c1ccc(-n2c3ccccc3c3cccc(N(c4cccc(-c5cccc6c5ccc5ccccc56)c4)c4cccc5ccccc45)c32)cc1 ; c1ccc(-n2c3ccccc3c3cccc(N(c4cccc(-c5cccc6ccccc56)c4)c4cccc(-c5cccc6c5ccc5ccccc56)c4)c32)cc1 ; c1ccc(-n2c3ccccc3c3cccc(N(c4cccc(-c5cccc6c5ccc5ccccc56)c4)c4cc5ccccc5c5ccccc45)c32)cc1]

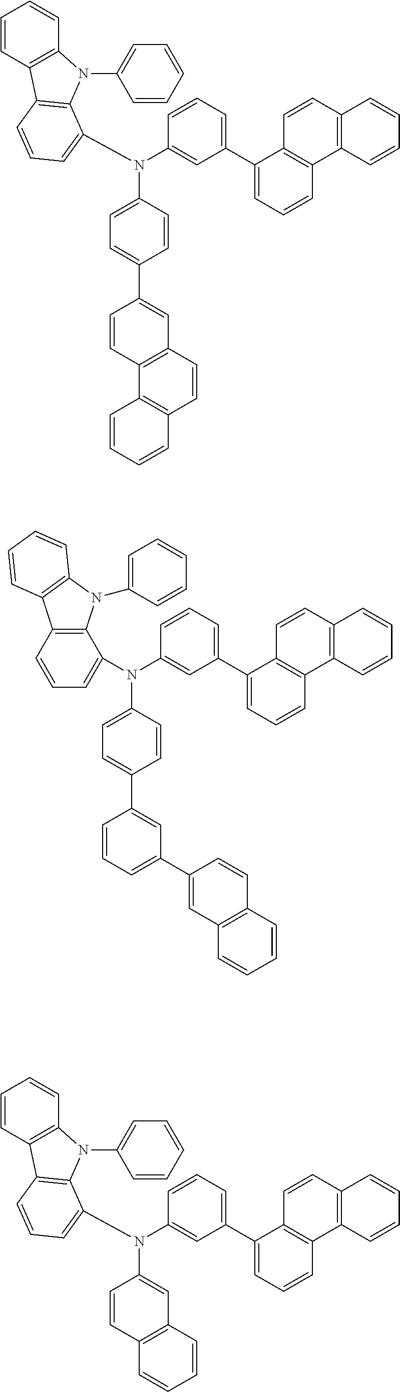 [SMILES: c1ccc(-n2c3ccccc3c3cccc(N(c4ccc(-c5ccc6c(ccc7ccccc76)c5)cc4)c4cccc(-c5cccc6c5ccc5ccccc56)c4)c32)cc1 ; c1ccc(-n2c3ccccc3c3cccc(N(c4ccc(-c5cccc(-c6ccc7ccccc7c6)c5)cc4)c4cccc(-c5cccc6c5ccc5ccccc56)c4)c32)cc1 ; c1ccc(-n2c3ccccc3c3cccc(N(c4cccc(-c5cccc6c5ccc5ccccc56)c4)c4ccc5ccccc5c4)c32)cc1]

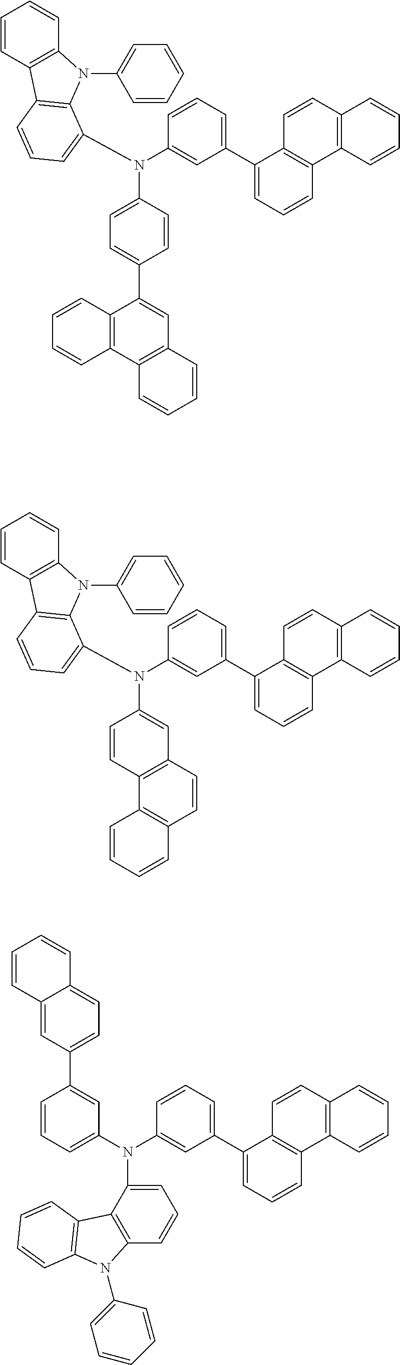 [SMILES: c1ccc(-n2c3ccccc3c3cccc(N(c4ccc(-c5cc6ccccc6c6ccccc56)cc4)c4cccc(-c5cccc6c5ccc5ccccc56)c4)c32)cc1 ; c1ccc(-n2c3ccccc3c3cccc(N(c4cccc(-c5cccc6c5ccc5ccccc56)c4)c4ccc5c(ccc6ccccc65)c4)c32)cc1 ; c1ccc(-n2c3ccccc3c3c(N(c4cccc(-c5ccc6ccccc6c5)c4)c4cccc(-c5cccc6c5ccc5ccccc56)c4)cccc32)cc1]

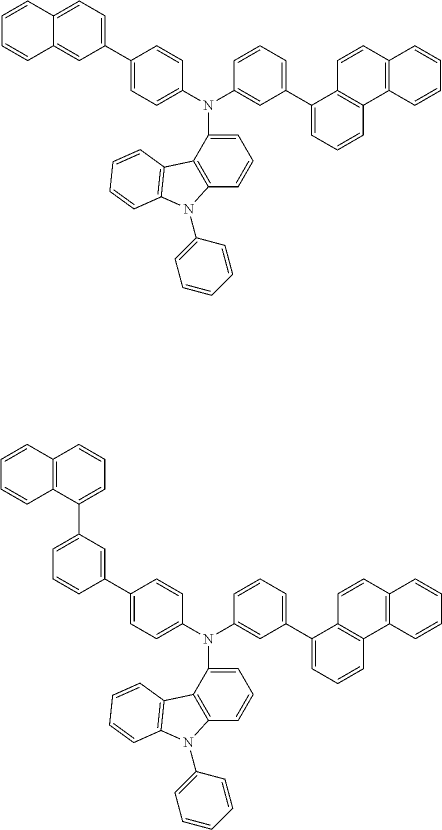 [SMILES: c1ccc(-n2c3ccccc3c3c(N(c4ccc(-c5ccc6ccccc6c5)cc4)c4cccc(-c5cccc6c5ccc5ccccc56)c4)cccc32)cc1 ; c1ccc(-n2c3ccccc3c3c(N(c4ccc(-c5cccc(-c6cccc7ccccc67)c5)cc4)c4cccc(-c5cccc6c5ccc5ccccc56)c4)cccc32)cc1]

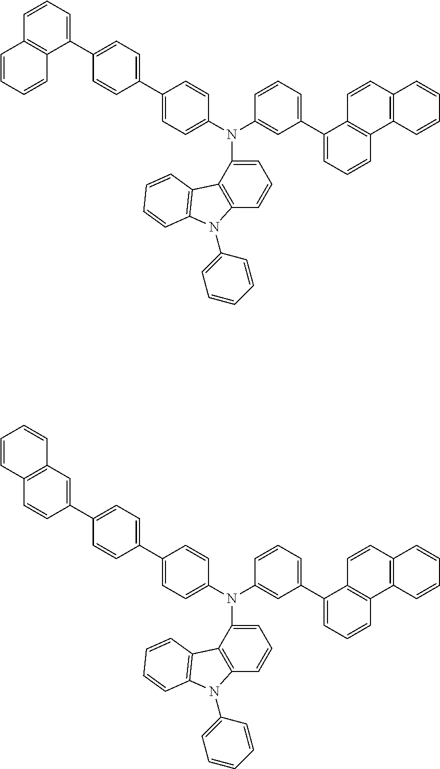 [SMILES: c1ccc(-n2c3ccccc3c3c(N(c4ccc(-c5ccc(-c6cccc7ccccc67)cc5)cc4)c4cccc(-c5cccc6c5ccc5ccccc56)c4)cccc32)cc1 ; c1ccc(-n2c3ccccc3c3c(N(c4ccc(-c5ccc(-c6ccc7ccccc7c6)cc5)cc4)c4cccc(-c5cccc6c5ccc5ccccc56)c4)cccc32)cc1]

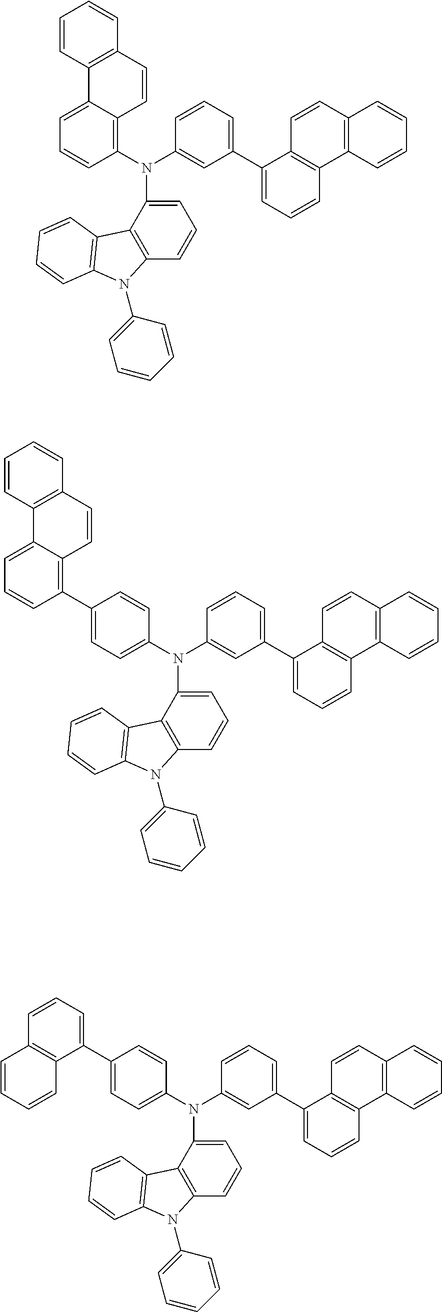 [SMILES: c1ccc(-n2c3ccccc3c3c(N(c4cccc(-c5cccc6c5ccc5ccccc56)c4)c4cccc5c4ccc4ccccc45)cccc32)cc1 ; c1ccc(-n2c3ccccc3c3c(N(c4ccc(-c5cccc6c5ccc5ccccc56)cc4)c4cccc(-c5cccc6c5ccc5ccccc56)c4)cccc32)cc1 ; c1ccc(-n2c3ccccc3c3c(N(c4ccc(-c5cccc6ccccc56)cc4)c4cccc(-c5cccc6c5ccc5ccccc56)c4)cccc32)cc1]

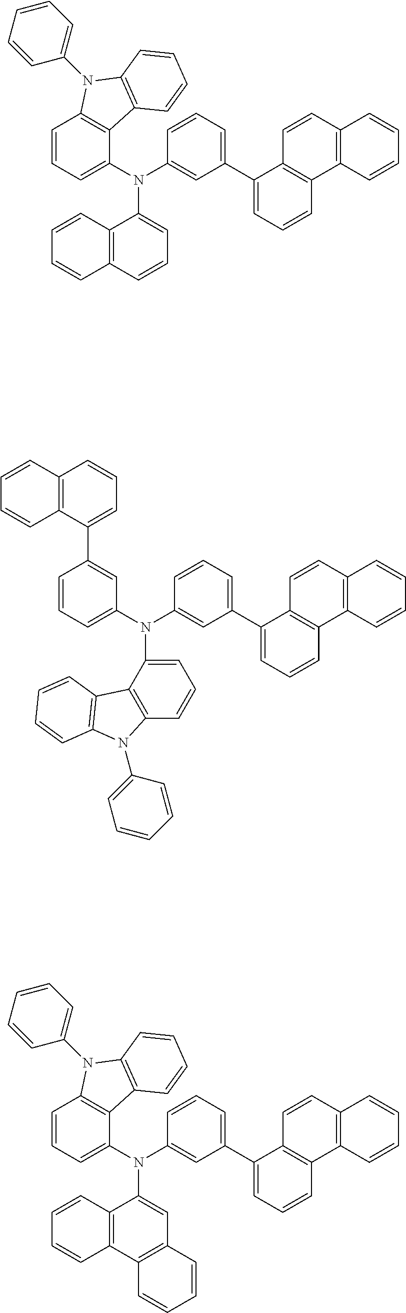 [SMILES: c1ccc(-n2c3ccccc3c3c(N(c4cccc(-c5cccc6c5ccc5ccccc56)c4)c4cccc5ccccc45)cccc32)cc1 ; c1ccc(-n2c3ccccc3c3c(N(c4cccc(-c5cccc6ccccc56)c4)c4cccc(-c5cccc6c5ccc5ccccc56)c4)cccc32)cc1 ; c1ccc(-n2c3ccccc3c3c(N(c4cccc(-c5cccc6c5ccc5ccccc56)c4)c4cc5ccccc5c5ccccc45)cccc32)cc1]

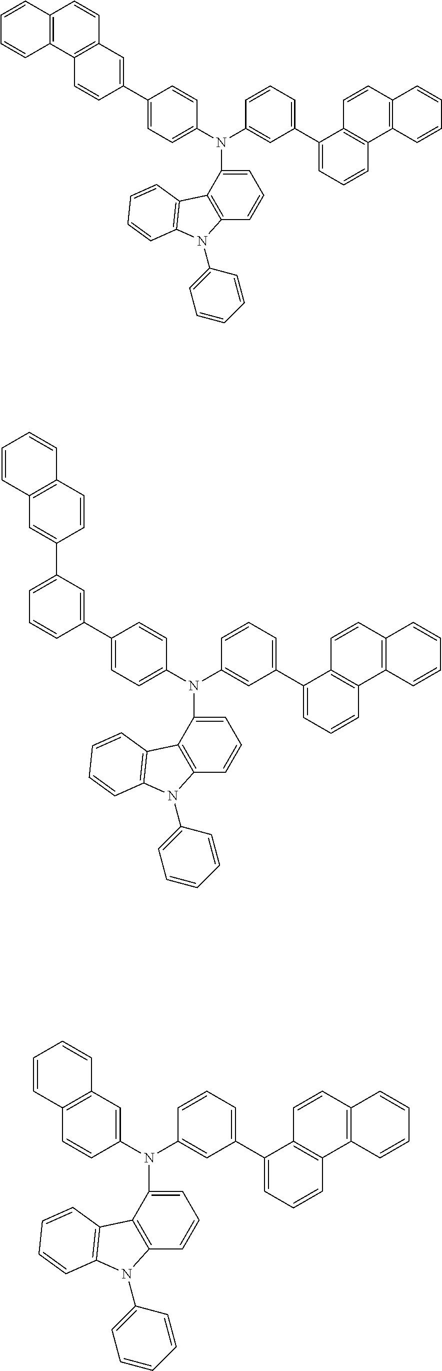 [SMILES: c1ccc(-n2c3ccccc3c3c(N(c4ccc(-c5ccc6c(ccc7ccccc76)c5)cc4)c4cccc(-c5cccc6c5ccc5ccccc56)c4)cccc32)cc1 ; c1ccc(-n2c3ccccc3c3c(N(c4ccc(-c5cccc(-c6ccc7ccccc7c6)c5)cc4)c4cccc(-c5cccc6c5ccc5ccccc56)c4)cccc32)cc1 ; c1ccc(-n2c3ccccc3c3c(N(c4cccc(-c5cccc6c5ccc5ccccc56)c4)c4ccc5ccccc5c4)cccc32)cc1]

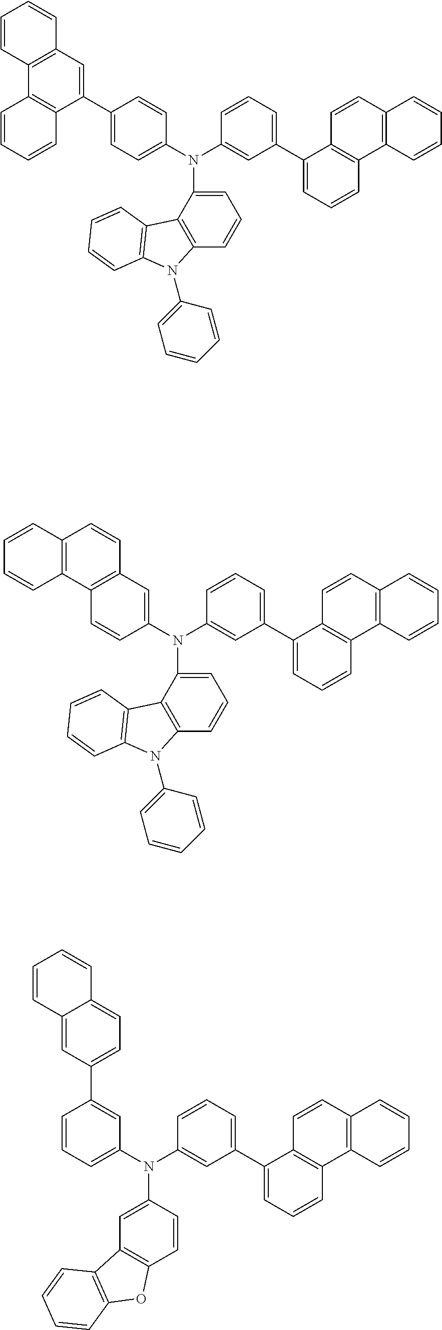 [SMILES: c1ccc(-n2c3ccccc3c3c(N(c4ccc(-c5cc6ccccc6c6ccccc56)cc4)c4cccc(-c5cccc6c5ccc5ccccc56)c4)cccc32)cc1 ; c1ccc(-n2c3ccccc3c3c(N(c4cccc(-c5cccc6c5ccc5ccccc56)c4)c4ccc5c(ccc6ccccc65)c4)cccc32)cc1 ; c1cc(-c2ccc3ccccc3c2)cc(N(c2cccc(-c3cccc4c3ccc3ccccc34)c2)c2ccc3oc4ccccc4c3c2)c1]

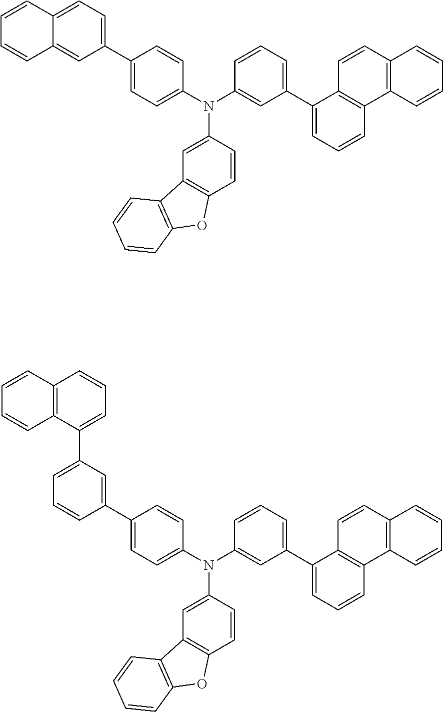 [SMILES: c1cc(-c2cccc3c2ccc2ccccc23)cc(N(c2ccc(-c3ccc4ccccc4c3)cc2)c2ccc3oc4ccccc4c3c2)c1 ; c1cc(-c2ccc(N(c3cccc(-c4cccc5c4ccc4ccccc45)c3)c3ccc4oc5ccccc5c4c3)cc2)cc(-c2cccc3ccccc23)c1]

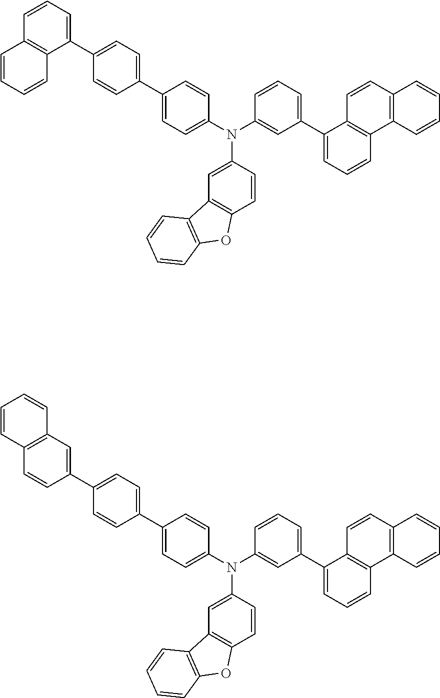 [SMILES: c1cc(-c2cccc3c2ccc2ccccc23)cc(N(c2ccc(-c3ccc(-c4cccc5ccccc45)cc3)cc2)c2ccc3oc4ccccc4c3c2)c1 ; c1cc(-c2cccc3c2ccc2ccccc23)cc(N(c2ccc(-c3ccc(-c4ccc5ccccc5c4)cc3)cc2)c2ccc3oc4ccccc4c3c2)c1]

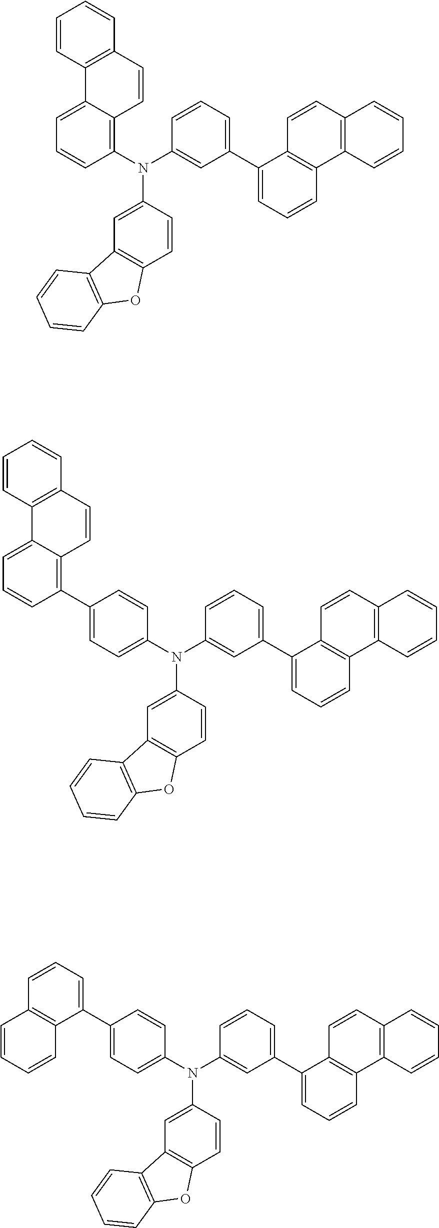 [SMILES: c1cc(-c2cccc3c2ccc2ccccc23)cc(N(c2ccc3oc4ccccc4c3c2)c2cccc3c2ccc2ccccc23)c1 ; c1cc(-c2cccc3c2ccc2ccccc23)cc(N(c2ccc(-c3cccc4c3ccc3ccccc34)cc2)c2ccc3oc4ccccc4c3c2)c1 ; c1cc(-c2cccc3c2ccc2ccccc23)cc(N(c2ccc(-c3cccc4ccccc34)cc2)c2ccc3oc4ccccc4c3c2)c1]

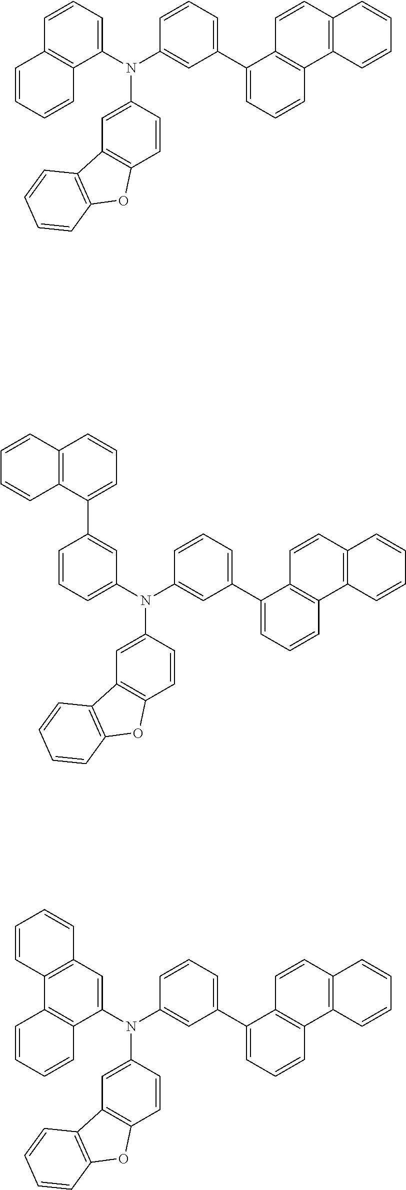 [SMILES: c1cc(-c2cccc3c2ccc2ccccc23)cc(N(c2ccc3oc4ccccc4c3c2)c2cccc3ccccc23)c1 ; c1cc(-c2cccc3ccccc23)cc(N(c2cccc(-c3cccc4c3ccc3ccccc34)c2)c2ccc3oc4ccccc4c3c2)c1 ; c1cc(-c2cccc3c2ccc2ccccc23)cc(N(c2ccc3oc4ccccc4c3c2)c2cc3ccccc3c3ccccc23)c1]

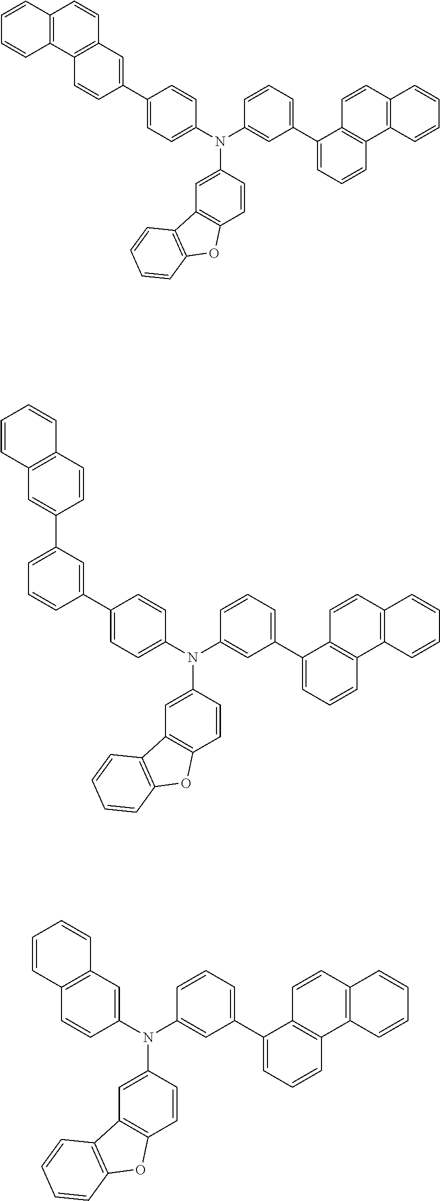 [SMILES: c1cc(-c2cccc3c2ccc2ccccc23)cc(N(c2ccc(-c3ccc4c(ccc5ccccc54)c3)cc2)c2ccc3oc4ccccc4c3c2)c1 ; c1cc(-c2ccc(N(c3cccc(-c4cccc5c4ccc4ccccc45)c3)c3ccc4oc5ccccc5c4c3)cc2)cc(-c2ccc3ccccc3c2)c1 ; c1cc(-c2cccc3c2ccc2ccccc23)cc(N(c2ccc3ccccc3c2)c2ccc3oc4ccccc4c3c2)c1]

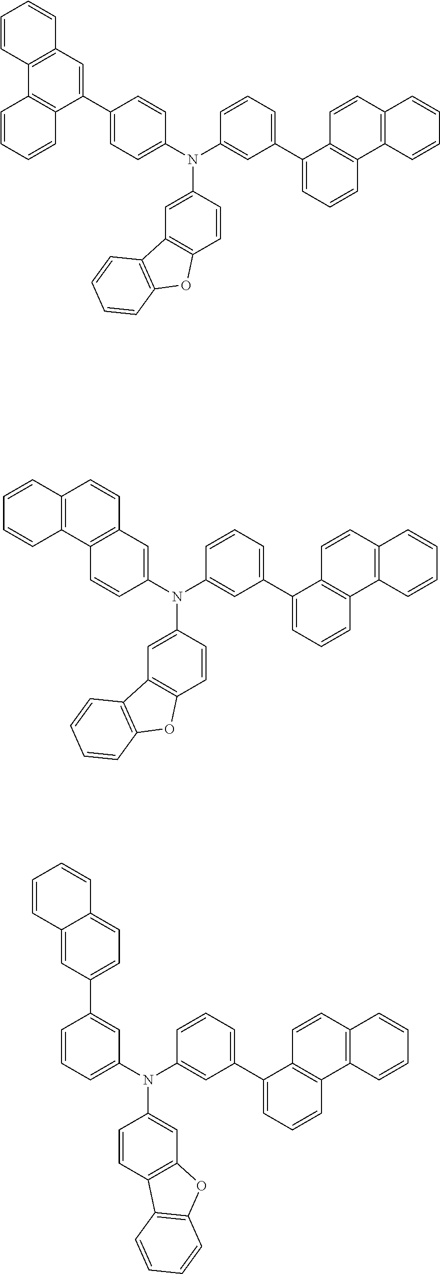 [SMILES: c1cc(-c2cccc3c2ccc2ccccc23)cc(N(c2ccc(-c3cc4ccccc4c4ccccc34)cc2)c2ccc3oc4ccccc4c3c2)c1 ; c1cc(-c2cccc3c2ccc2ccccc23)cc(N(c2ccc3c(ccc4ccccc43)c2)c2ccc3oc4ccccc4c3c2)c1 ; c1cc(-c2ccc3ccccc3c2)cc(N(c2cccc(-c3cccc4c3ccc3ccccc34)c2)c2ccc3c(c2)oc2ccccc23)c1]

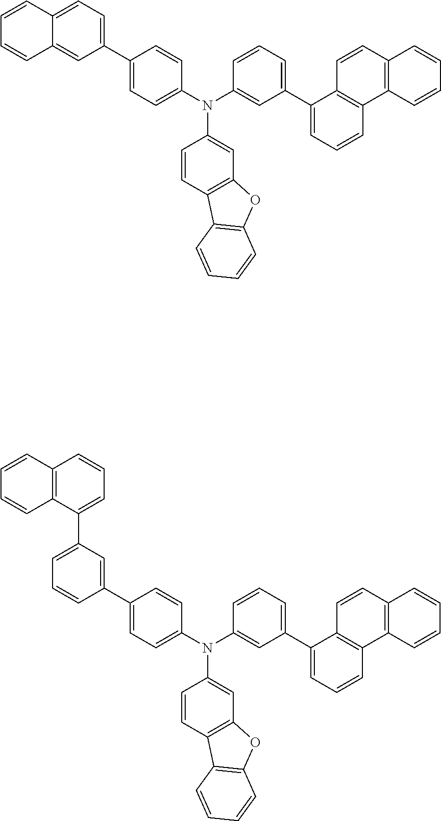 [SMILES: c1cc(-c2cccc3c2ccc2ccccc23)cc(N(c2ccc(-c3ccc4ccccc4c3)cc2)c2ccc3c(c2)oc2ccccc23)c1 ; c1cc(-c2ccc(N(c3cccc(-c4cccc5c4ccc4ccccc45)c3)c3ccc4c(c3)oc3ccccc34)cc2)cc(-c2cccc3ccccc23)c1]

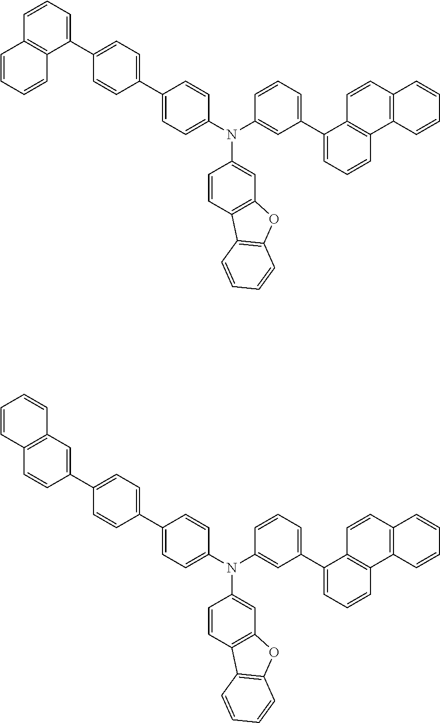 [SMILES: c1cc(-c2cccc3c2ccc2ccccc23)cc(N(c2ccc(-c3ccc(-c4cccc5ccccc45)cc3)cc2)c2ccc3c(c2)oc2ccccc23)c1 ; c1cc(-c2cccc3c2ccc2ccccc23)cc(N(c2ccc(-c3ccc(-c4ccc5ccccc5c4)cc3)cc2)c2ccc3c(c2)oc2ccccc23)c1]

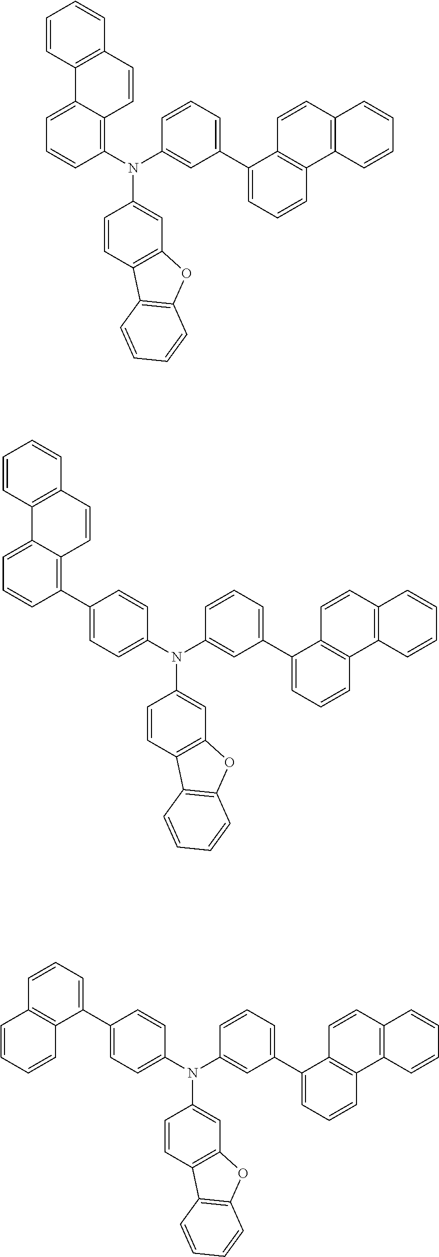 [SMILES: c1cc(-c2cccc3c2ccc2ccccc23)cc(N(c2ccc3c(c2)oc2ccccc23)c2cccc3c2ccc2ccccc23)c1 ; c1cc(-c2cccc3c2ccc2ccccc23)cc(N(c2ccc(-c3cccc4c3ccc3ccccc34)cc2)c2ccc3c(c2)oc2ccccc23)c1 ; c1cc(-c2cccc3c2ccc2ccccc23)cc(N(c2ccc(-c3cccc4ccccc34)cc2)c2ccc3c(c2)oc2ccccc23)c1]

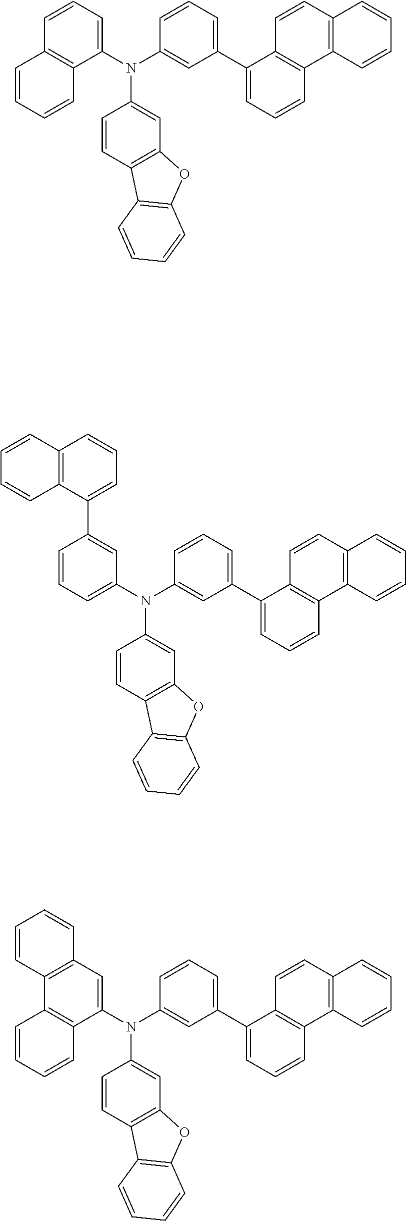 [SMILES: c1cc(-c2cccc3c2ccc2ccccc23)cc(N(c2ccc3c(c2)oc2ccccc23)c2cccc3ccccc23)c1 ; c1cc(-c2cccc3ccccc23)cc(N(c2cccc(-c3cccc4c3ccc3ccccc34)c2)c2ccc3c(c2)oc2ccccc23)c1 ; c1cc(-c2cccc3c2ccc2ccccc23)cc(N(c2ccc3c(c2)oc2ccccc23)c2cc3ccccc3c3ccccc23)c1]

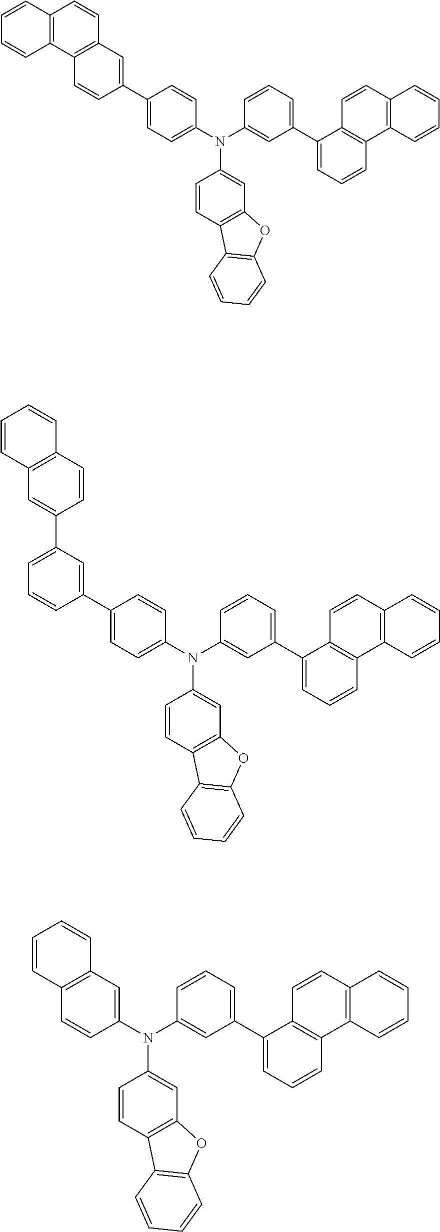 [SMILES: c1cc(-c2cccc3c2ccc2ccccc23)cc(N(c2ccc(-c3ccc4c(ccc5ccccc54)c3)cc2)c2ccc3c(c2)oc2ccccc23)c1 ; c1cc(-c2ccc(N(c3cccc(-c4cccc5c4ccc4ccccc45)c3)c3ccc4c(c3)oc3ccccc34)cc2)cc(-c2ccc3ccccc3c2)c1 ; c1cc(-c2cccc3c2ccc2ccccc23)cc(N(c2ccc3ccccc3c2)c2ccc3c(c2)oc2ccccc23)c1]

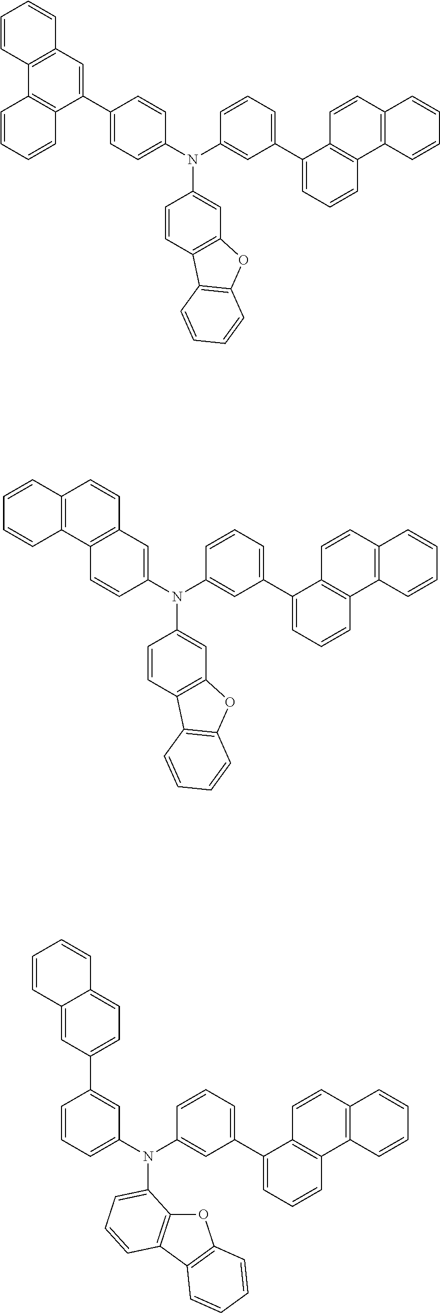 [SMILES: c1cc(-c2cccc3c2ccc2ccccc23)cc(N(c2ccc(-c3cc4ccccc4c4ccccc34)cc2)c2ccc3c(c2)oc2ccccc23)c1 ; c1cc(-c2cccc3c2ccc2ccccc23)cc(N(c2ccc3c(ccc4ccccc43)c2)c2ccc3c(c2)oc2ccccc23)c1 ; c1cc(-c2ccc3ccccc3c2)cc(N(c2cccc(-c3cccc4c3ccc3ccccc34)c2)c2cccc3c2oc2ccccc23)c1]

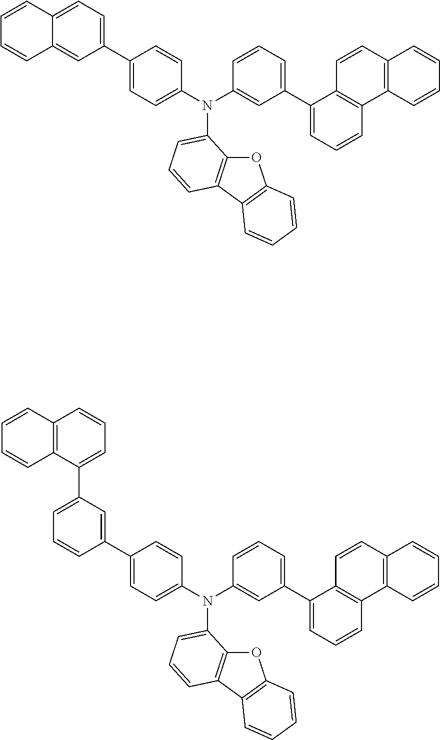 [SMILES: c1cc(-c2cccc3c2ccc2ccccc23)cc(N(c2ccc(-c3ccc4ccccc4c3)cc2)c2cccc3c2oc2ccccc23)c1 ; c1cc(-c2ccc(N(c3cccc(-c4cccc5c4ccc4ccccc45)c3)c3cccc4c3oc3ccccc34)cc2)cc(-c2cccc3ccccc23)c1]

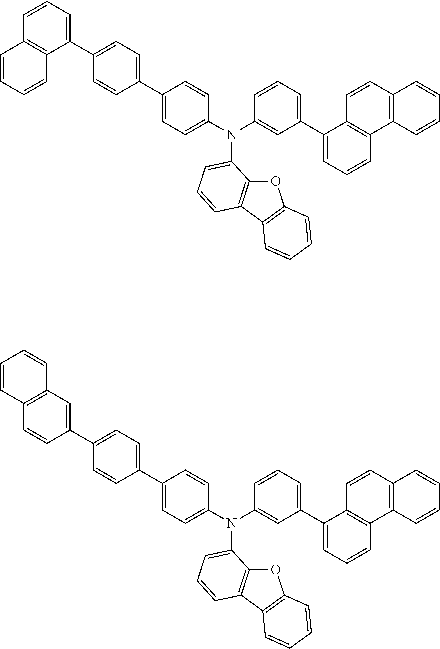 [SMILES: c1cc(-c2cccc3c2ccc2ccccc23)cc(N(c2ccc(-c3ccc(-c4cccc5ccccc45)cc3)cc2)c2cccc3c2oc2ccccc23)c1 ; c1cc(-c2cccc3c2ccc2ccccc23)cc(N(c2ccc(-c3ccc(-c4ccc5ccccc5c4)cc3)cc2)c2cccc3c2oc2ccccc23)c1]

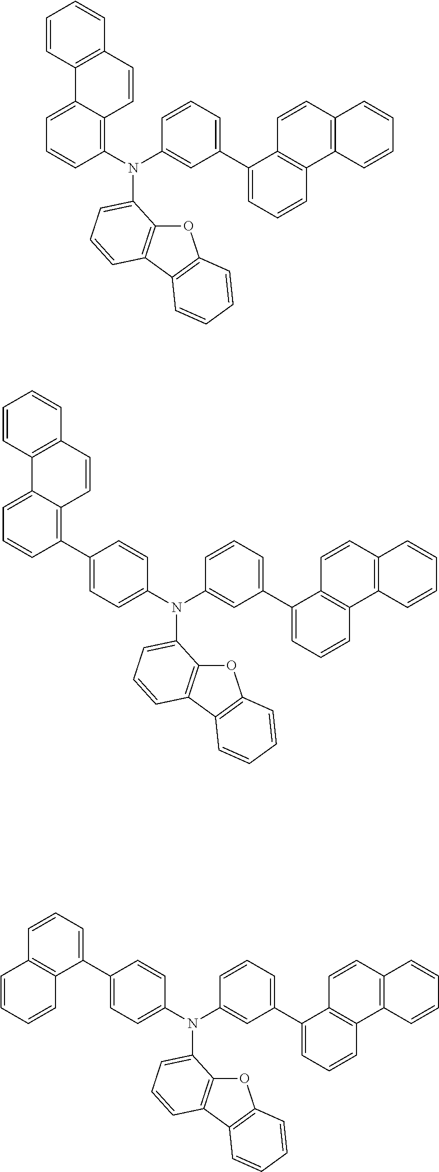 [SMILES: c1cc(-c2cccc3c2ccc2ccccc23)cc(N(c2cccc3c2ccc2ccccc23)c2cccc3c2oc2ccccc23)c1 ; c1cc(-c2cccc3c2ccc2ccccc23)cc(N(c2ccc(-c3cccc4c3ccc3ccccc34)cc2)c2cccc3c2oc2ccccc23)c1 ; c1cc(-c2cccc3c2ccc2ccccc23)cc(N(c2ccc(-c3cccc4ccccc34)cc2)c2cccc3c2oc2ccccc23)c1]

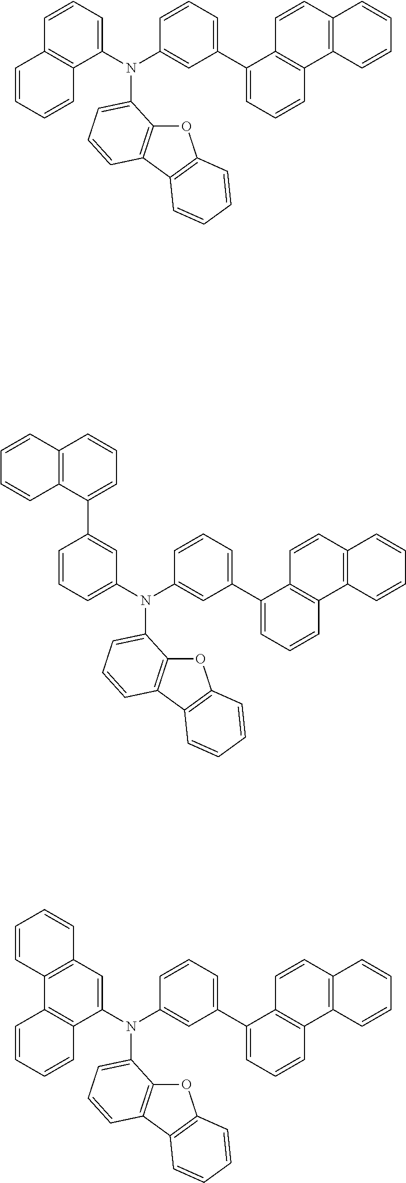 [SMILES: c1cc(-c2cccc3c2ccc2ccccc23)cc(N(c2cccc3ccccc23)c2cccc3c2oc2ccccc23)c1 ; c1cc(-c2cccc3ccccc23)cc(N(c2cccc(-c3cccc4c3ccc3ccccc34)c2)c2cccc3c2oc2ccccc23)c1 ; c1cc(-c2cccc3c2ccc2ccccc23)cc(N(c2cc3ccccc3c3ccccc23)c2cccc3c2oc2ccccc23)c1]

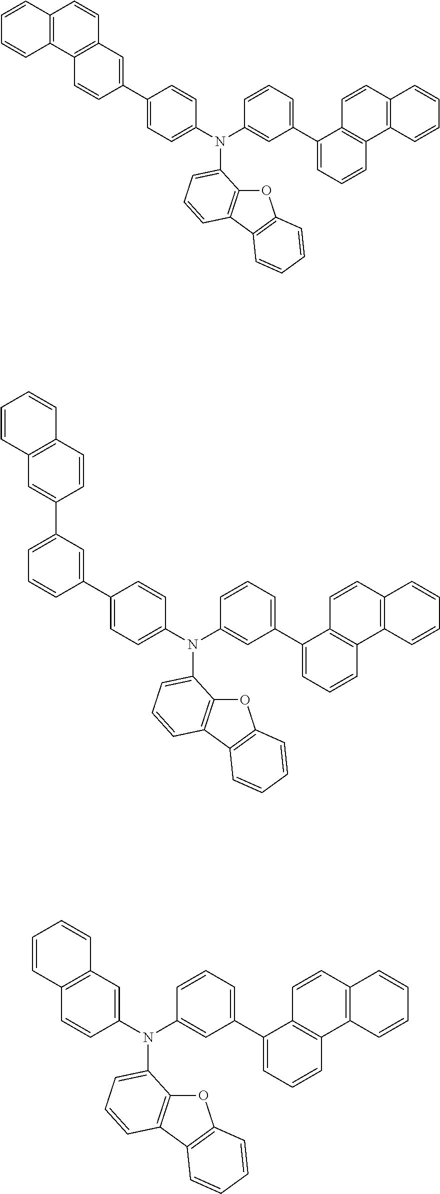 [SMILES: c1cc(-c2cccc3c2ccc2ccccc23)cc(N(c2ccc(-c3ccc4c(ccc5ccccc54)c3)cc2)c2cccc3c2oc2ccccc23)c1 ; c1cc(-c2ccc(N(c3cccc(-c4cccc5c4ccc4ccccc45)c3)c3cccc4c3oc3ccccc34)cc2)cc(-c2ccc3ccccc3c2)c1 ; c1cc(-c2cccc3c2ccc2ccccc23)cc(N(c2ccc3ccccc3c2)c2cccc3c2oc2ccccc23)c1]

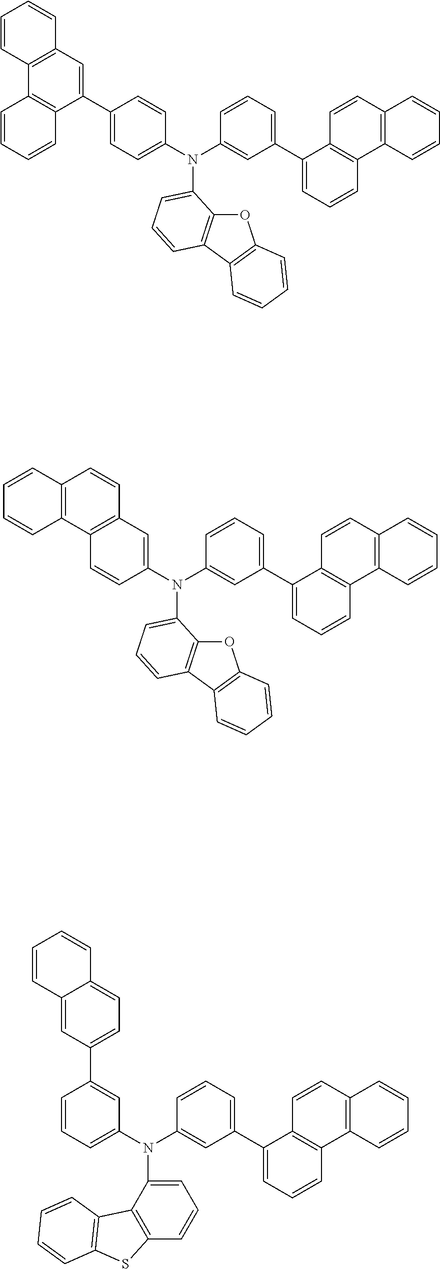 [SMILES: c1cc(-c2cccc3c2ccc2ccccc23)cc(N(c2ccc(-c3cc4ccccc4c4ccccc34)cc2)c2cccc3c2oc2ccccc23)c1 ; c1cc(-c2cccc3c2ccc2ccccc23)cc(N(c2ccc3c(ccc4ccccc43)c2)c2cccc3c2oc2ccccc23)c1 ; c1cc(-c2ccc3ccccc3c2)cc(N(c2cccc(-c3cccc4c3ccc3ccccc34)c2)c2cccc3sc4ccccc4c23)c1]

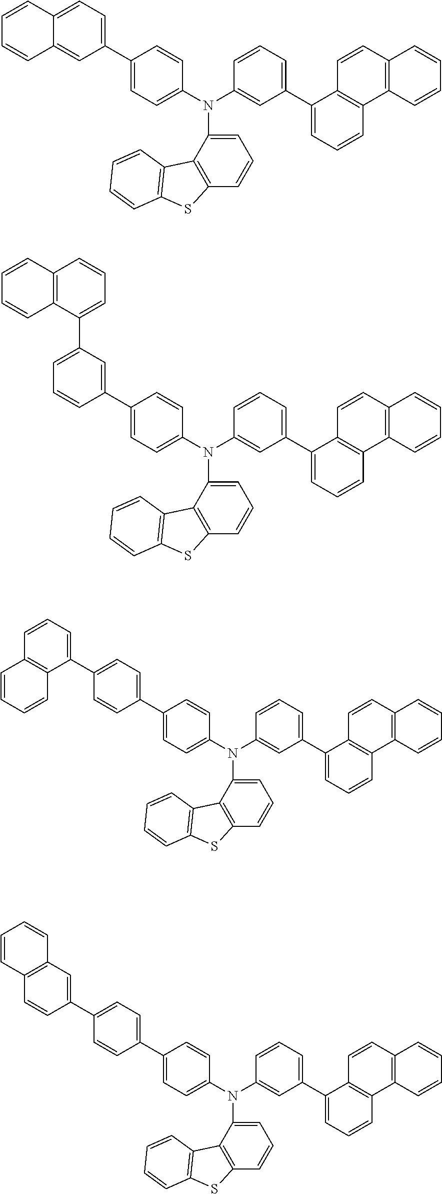 [SMILES: c1cc(-c2cccc3c2ccc2ccccc23)cc(N(c2ccc(-c3ccc4ccccc4c3)cc2)c2cccc3sc4ccccc4c23)c1 ; c1cc(-c2ccc(N(c3cccc(-c4cccc5c4ccc4ccccc45)c3)c3cccc4sc5ccccc5c34)cc2)cc(-c2cccc3ccccc23)c1 ; c1cc(-c2cccc3c2ccc2ccccc23)cc(N(c2ccc(-c3ccc(-c4cccc5ccccc45)cc3)cc2)c2cccc3sc4ccccc4c23)c1 ; c1cc(-c2cccc3c2ccc2ccccc23)cc(N(c2ccc(-c3ccc(-c4ccc5ccccc5c4)cc3)cc2)c2cccc3sc4ccccc4c23)c1]

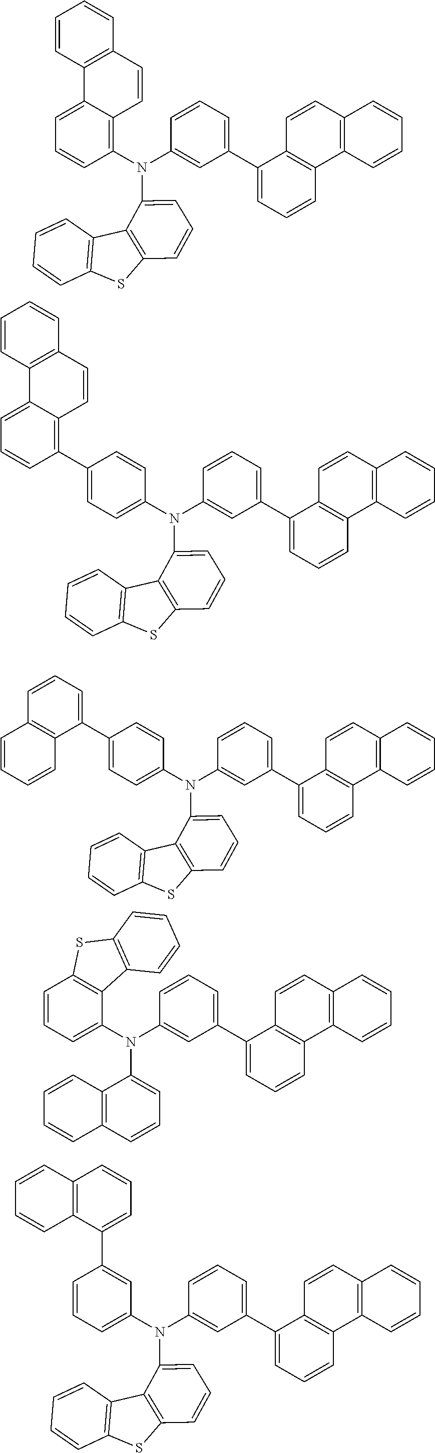

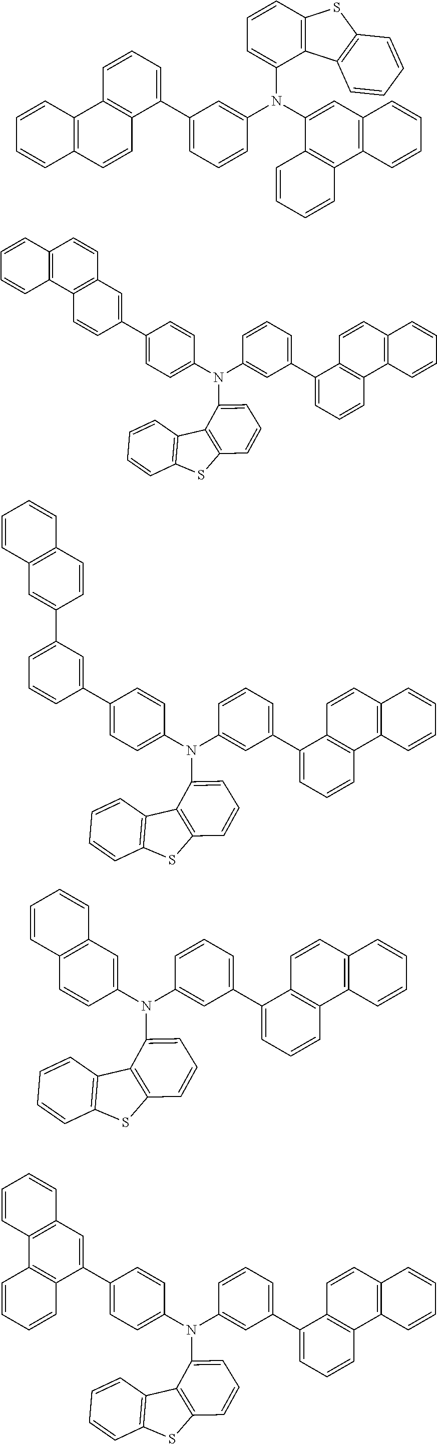 [SMILES: c1cc(-c2cccc3c2ccc2ccccc23)cc(N(c2cc3ccccc3c3ccccc23)c2cccc3sc4ccccc4c23)c1 ; c1cc(-c2cccc3c2ccc2ccccc23)cc(N(c2ccc(-c3ccc4c(ccc5ccccc54)c3)cc2)c2cccc3sc4ccccc4c23)c1 ; c1cc(-c2ccc(N(c3cccc(-c4cccc5c4ccc4ccccc45)c3)c3cccc4sc5ccccc5c34)cc2)cc(-c2ccc3ccccc3c2)c1 ; c1cc(-c2cccc3c2ccc2ccccc23)cc(N(c2ccc3ccccc3c2)c2cccc3sc4ccccc4c23)c1 ; c1cc(-c2cccc3c2ccc2ccccc23)cc(N(c2ccc(-c3cc4ccccc4c4ccccc34)cc2)c2cccc3sc4ccccc4c23)c1]

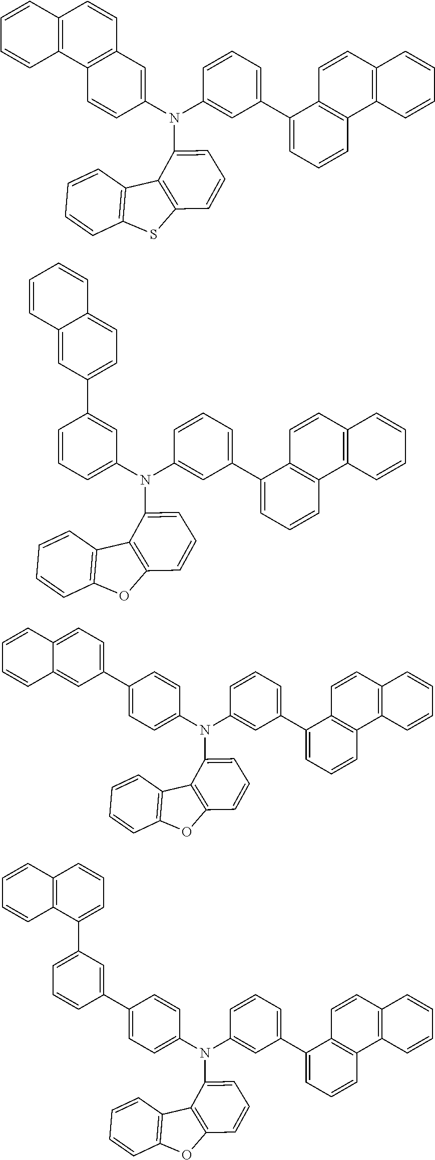

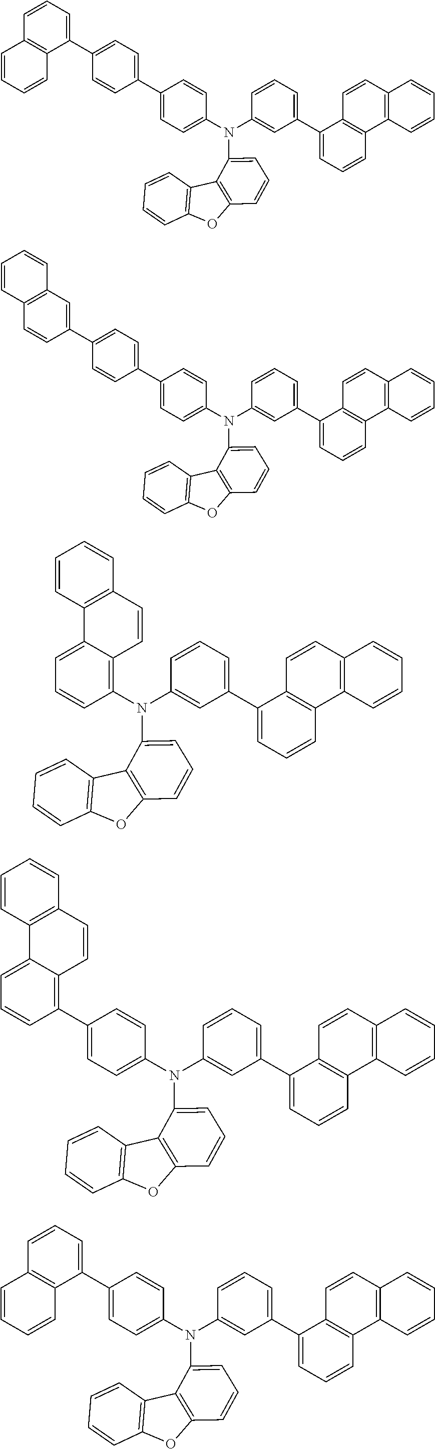 [SMILES: c1cc(-c2cccc3c2ccc2ccccc23)cc(N(c2ccc(-c3ccc(-c4cccc5ccccc45)cc3)cc2)c2cccc3oc4ccccc4c23)c1 ; c1cc(-c2cccc3c2ccc2ccccc23)cc(N(c2ccc(-c3ccc(-c4ccc5ccccc5c4)cc3)cc2)c2cccc3oc4ccccc4c23)c1 ; c1cc(-c2cccc3c2ccc2ccccc23)cc(N(c2cccc3c2ccc2ccccc23)c2cccc3oc4ccccc4c23)c1 ; c1cc(-c2cccc3c2ccc2ccccc23)cc(N(c2ccc(-c3cccc4c3ccc3ccccc34)cc2)c2cccc3oc4ccccc4c23)c1 ; c1cc(-c2cccc3c2ccc2ccccc23)cc(N(c2ccc(-c3cccc4ccccc34)cc2)c2cccc3oc4ccccc4c23)c1]

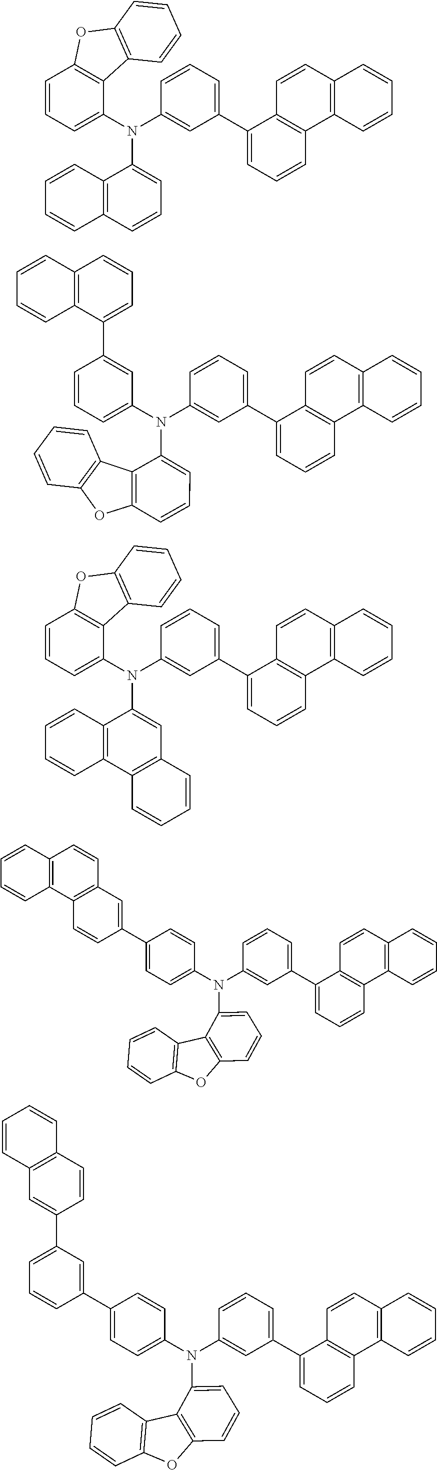

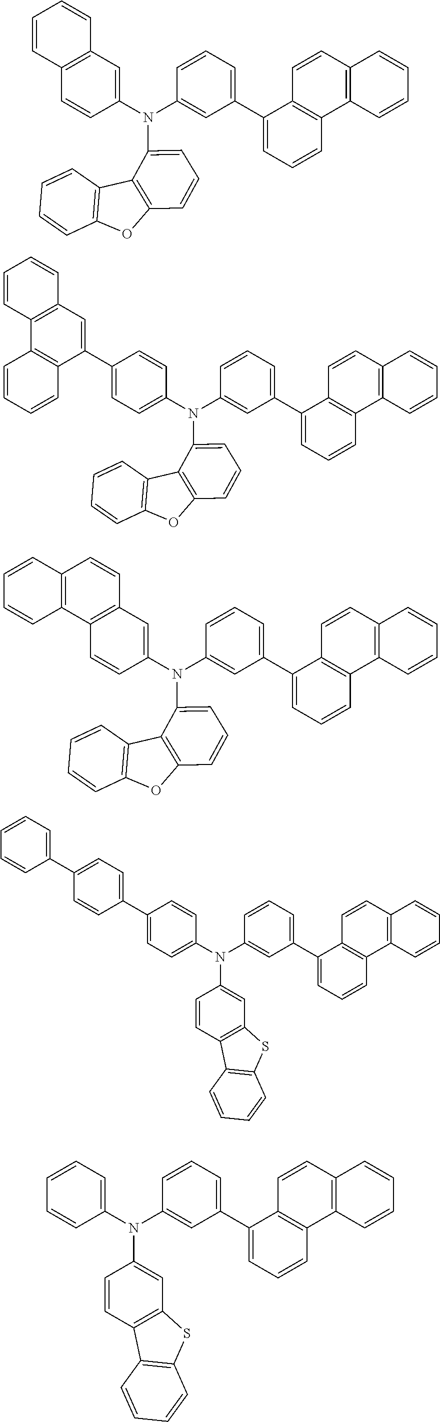

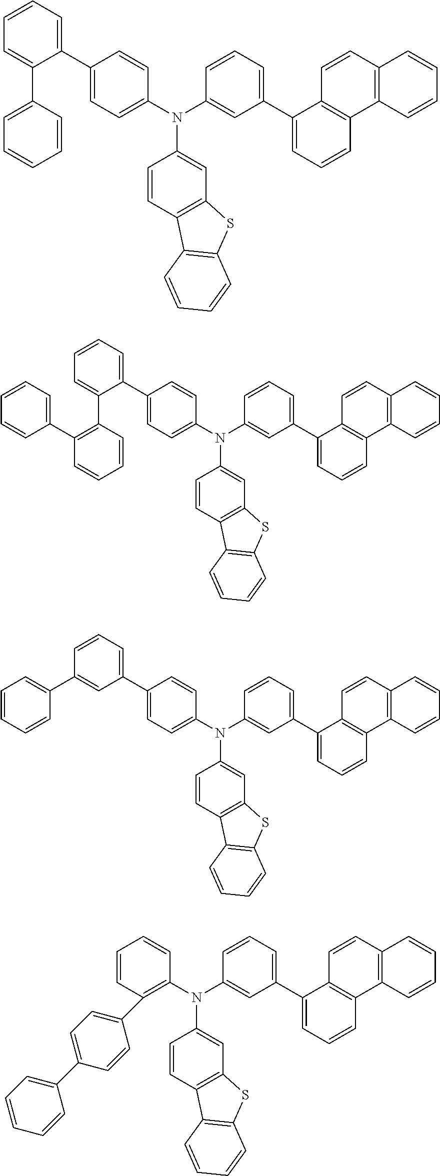 [SMILES: c1ccc(-c2ccccc2-c2ccc(N(c3cccc(-c4cccc5c4ccc4ccccc45)c3)c3ccc4c(c3)sc3ccccc34)cc2)cc1 ; c1ccc(-c2ccccc2-c2ccccc2-c2ccc(N(c3cccc(-c4cccc5c4ccc4ccccc45)c3)c3ccc4c(c3)sc3ccccc34)cc2)cc1 ; c1ccc(-c2cccc(-c3ccc(N(c4cccc(-c5cccc6c5ccc5ccccc56)c4)c4ccc5c(c4)sc4ccccc45)cc3)c2)cc1 ; c1ccc(-c2ccc(-c3ccccc3N(c3cccc(-c4cccc5c4ccc4ccccc45)c3)c3ccc4c(c3)sc3ccccc34)cc2)cc1]

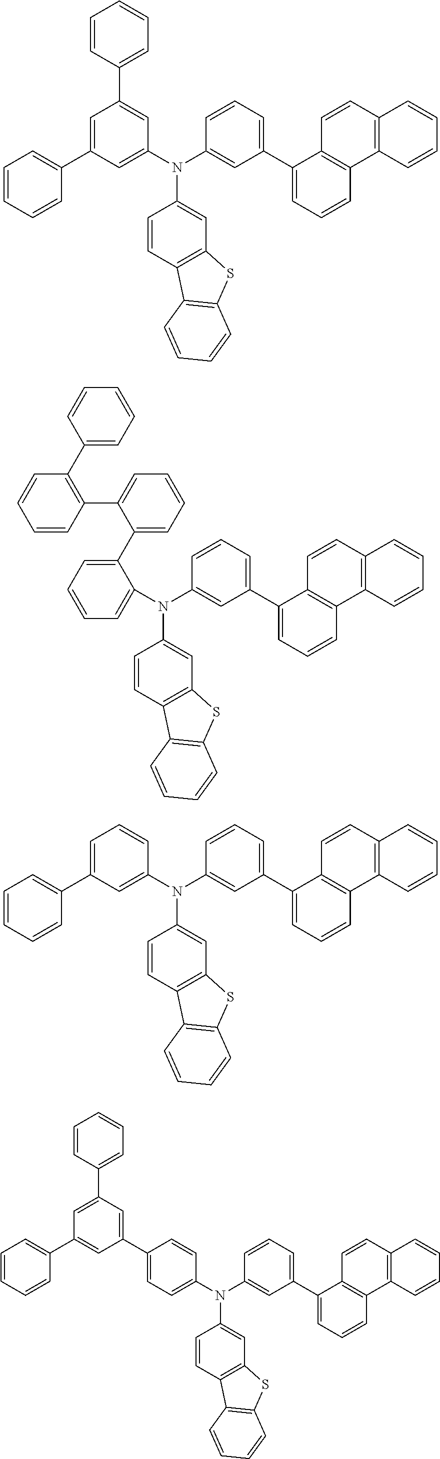

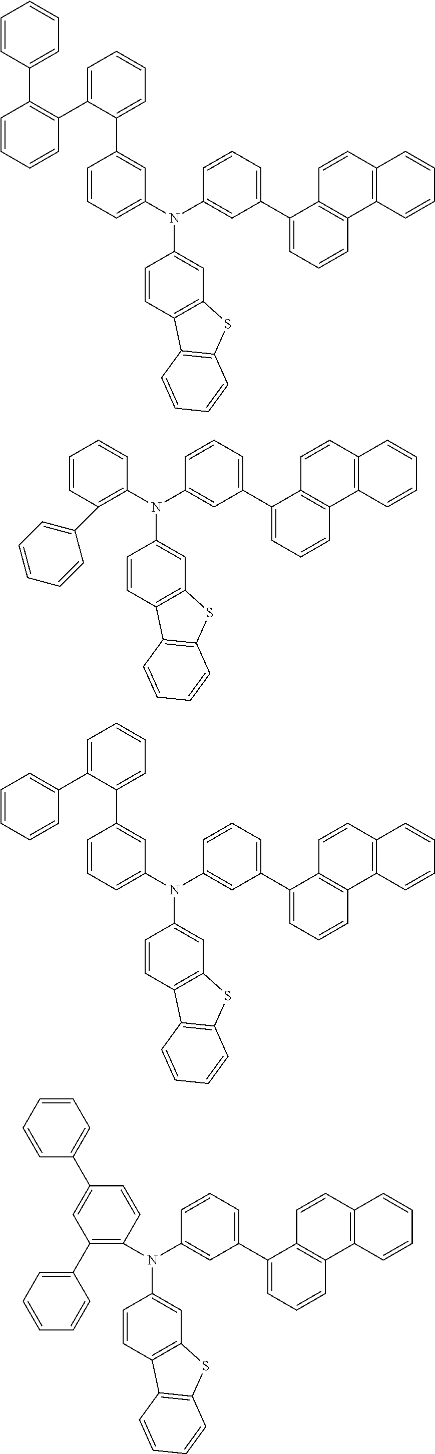

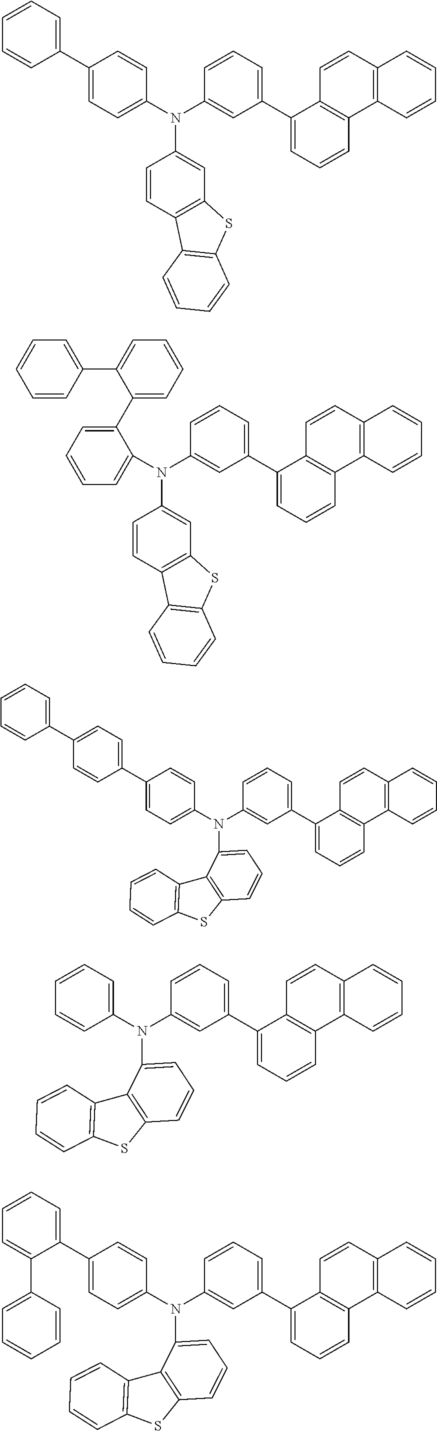

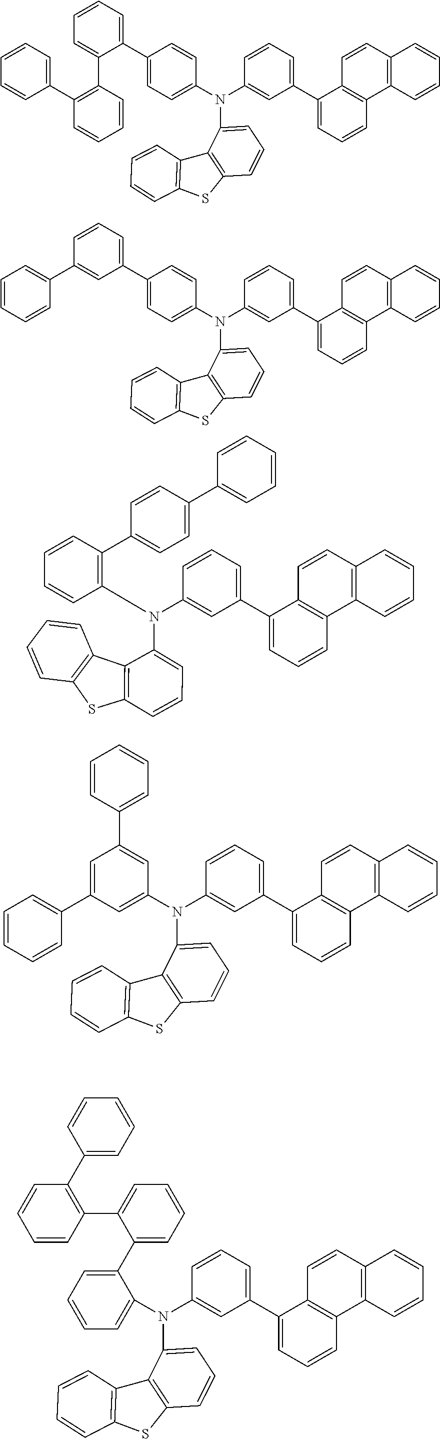

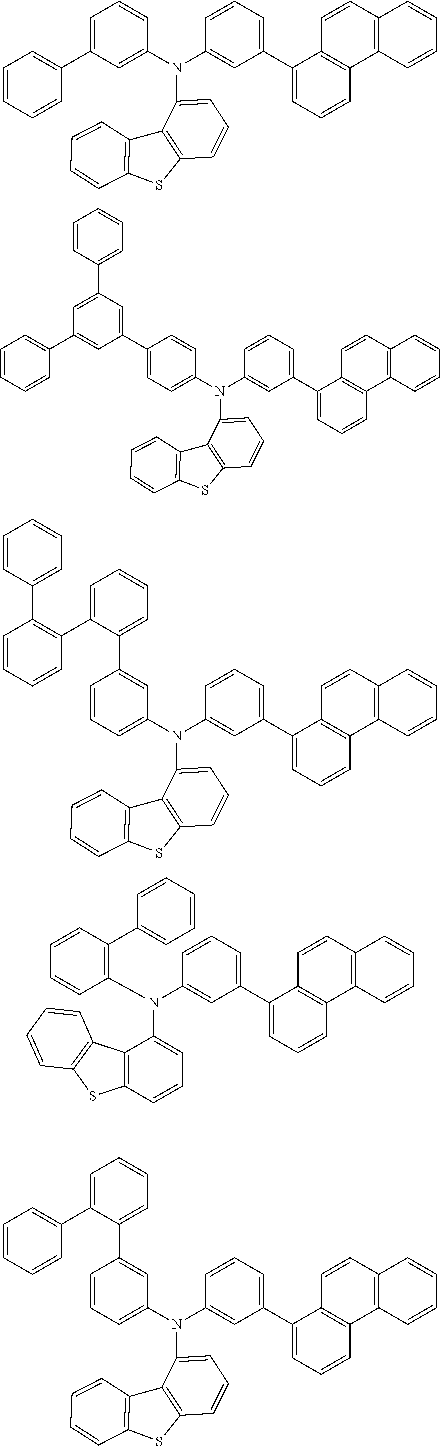

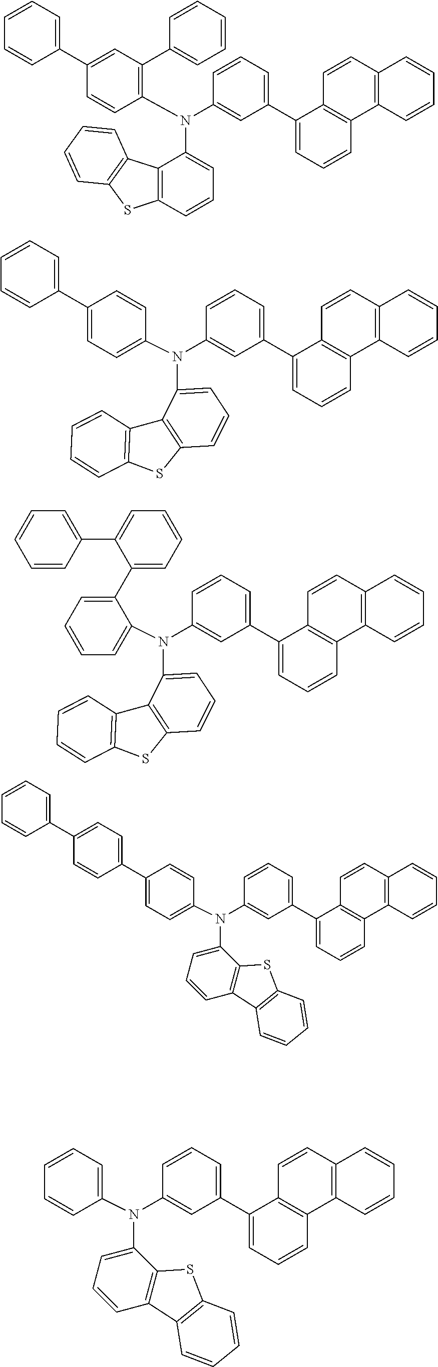

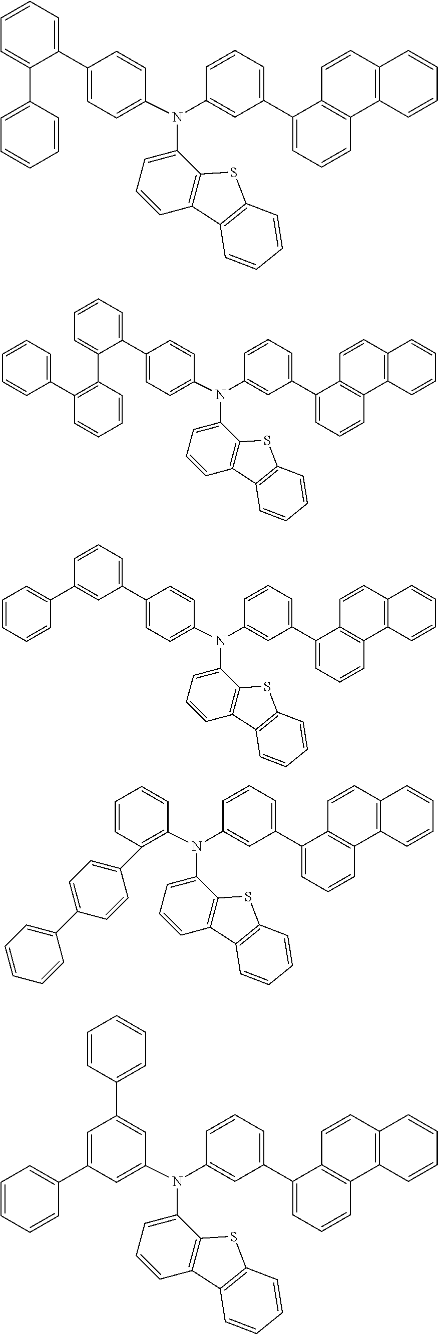

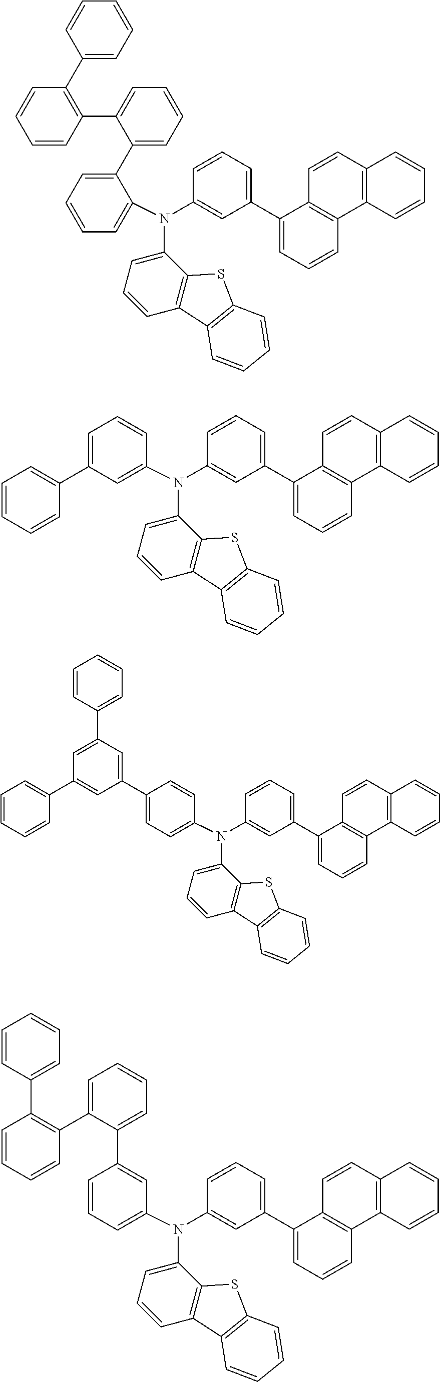

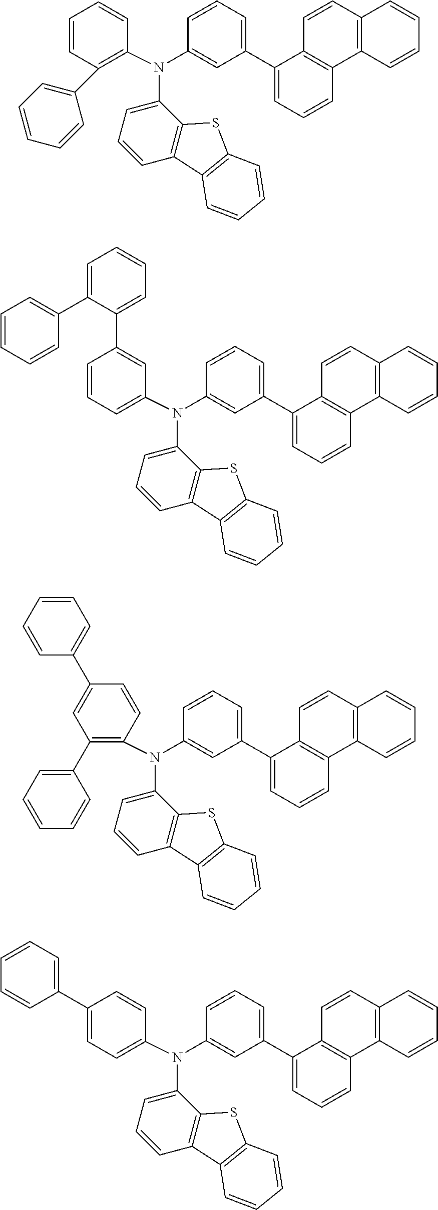 [SMILES: c1ccc(-c2ccccc2N(c2cccc(-c3cccc4c3ccc3ccccc34)c2)c2cccc3c2sc2ccccc23)cc1 ; c1ccc(-c2ccccc2-c2cccc(N(c3cccc(-c4cccc5c4ccc4ccccc45)c3)c3cccc4c3sc3ccccc34)c2)cc1 ; c1ccc(-c2ccc(N(c3cccc(-c4cccc5c4ccc4ccccc45)c3)c3cccc4c3sc3ccccc34)c(-c3ccccc3)c2)cc1 ; c1ccc(-c2ccc(N(c3cccc(-c4cccc5c4ccc4ccccc45)c3)c3cccc4c3sc3ccccc34)cc2)cc1]

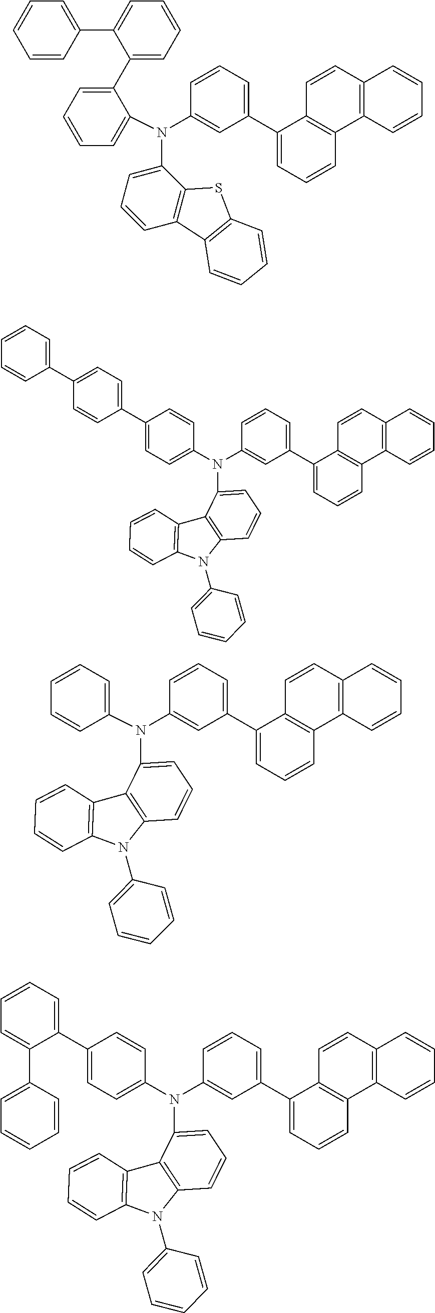

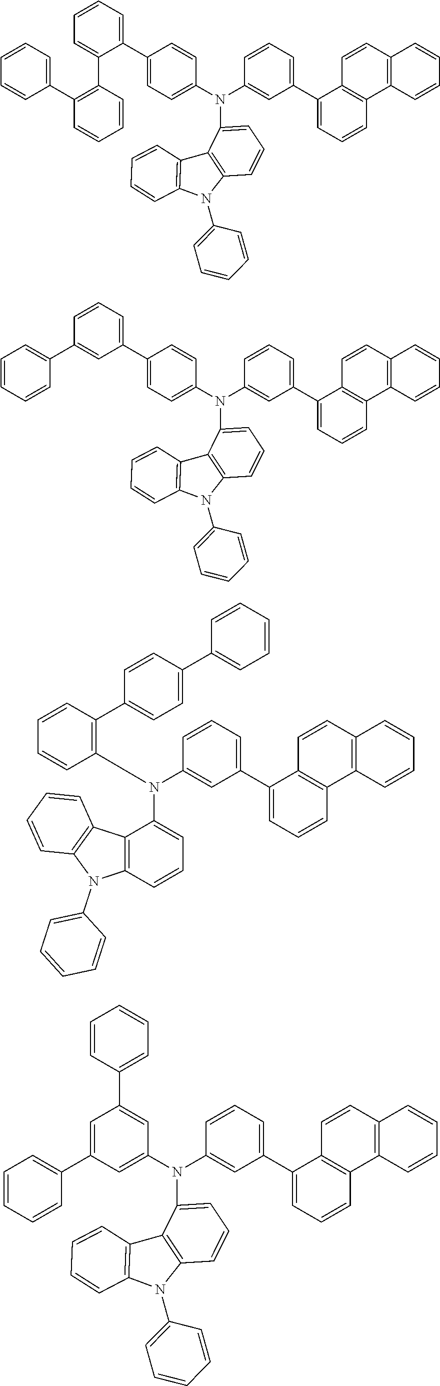

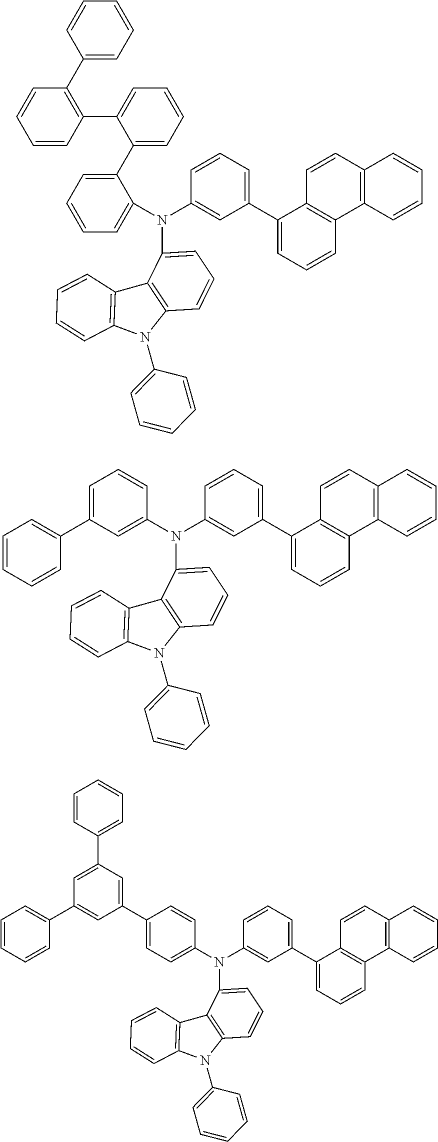 [SMILES: c1ccc(-c2ccccc2-c2ccccc2-c2ccccc2N(c2cccc(-c3cccc4c3ccc3ccccc34)c2)c2cccc3c2c2ccccc2n3-c2ccccc2)cc1 ; c1ccc(-c2cccc(N(c3cccc(-c4cccc5c4ccc4ccccc45)c3)c3cccc4c3c3ccccc3n4-c3ccccc3)c2)cc1 ; c1ccc(-c2cc(-c3ccccc3)cc(-c3ccc(N(c4cccc(-c5cccc6c5ccc5ccccc56)c4)c4cccc5c4c4ccccc4n5-c4ccccc4)cc3)c2)cc1]

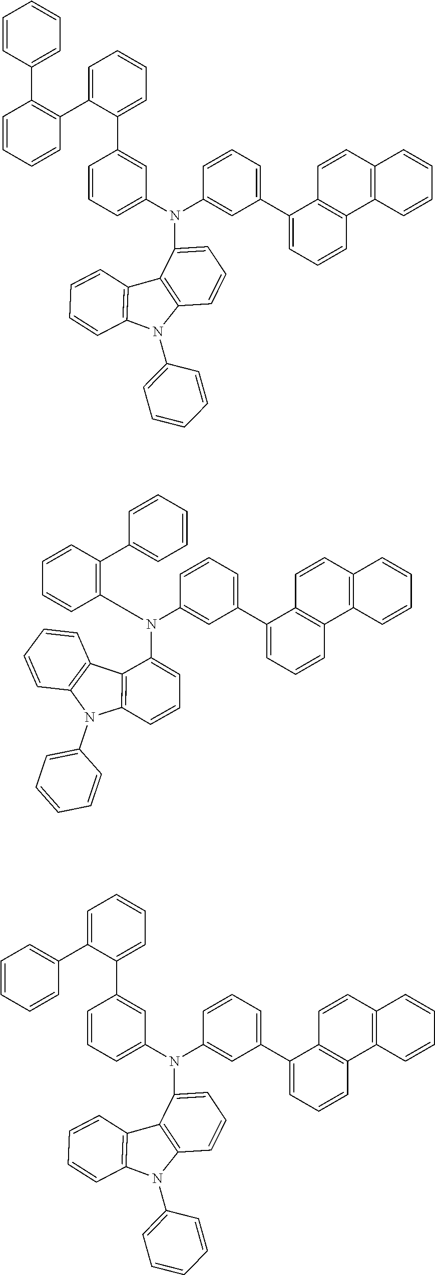 [SMILES: c1ccc(-c2ccccc2-c2ccccc2-c2cccc(N(c3cccc(-c4cccc5c4ccc4ccccc45)c3)c3cccc4c3c3ccccc3n4-c3ccccc3)c2)cc1 ; c1ccc(-c2ccccc2N(c2cccc(-c3cccc4c3ccc3ccccc34)c2)c2cccc3c2c2ccccc2n3-c2ccccc2)cc1 ; c1ccc(-c2ccccc2-c2cccc(N(c3cccc(-c4cccc5c4ccc4ccccc45)c3)c3cccc4c3c3ccccc3n4-c3ccccc3)c2)cc1]

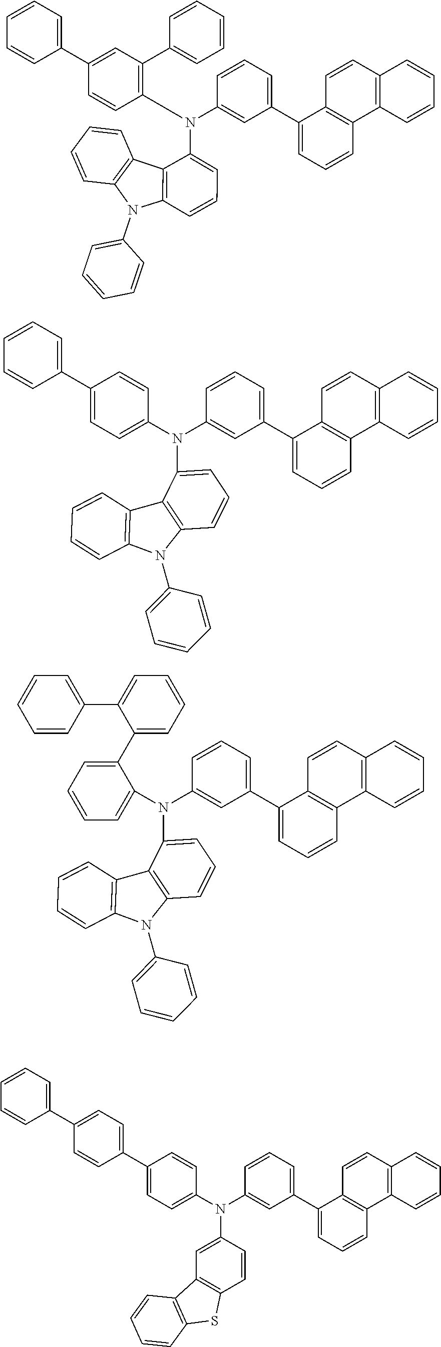

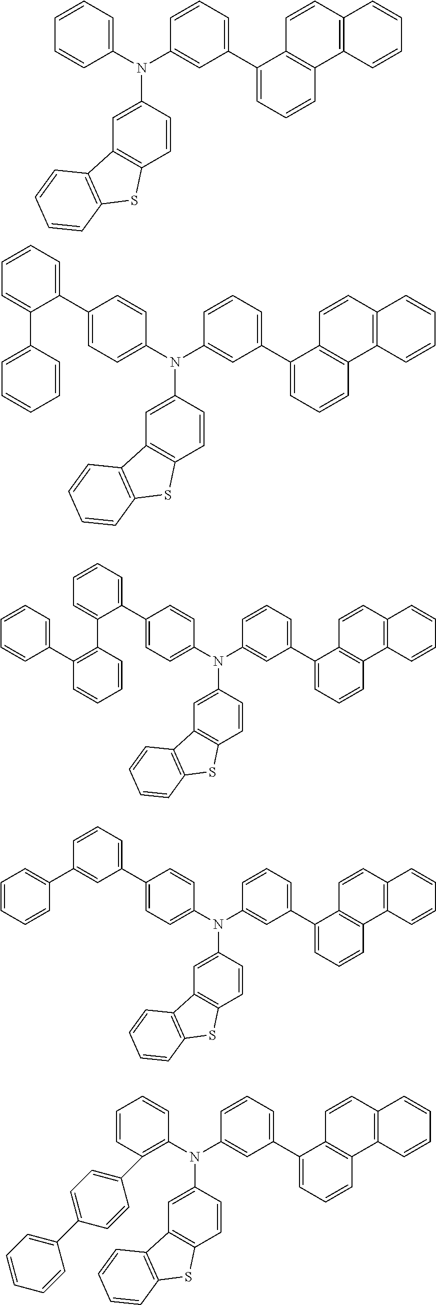

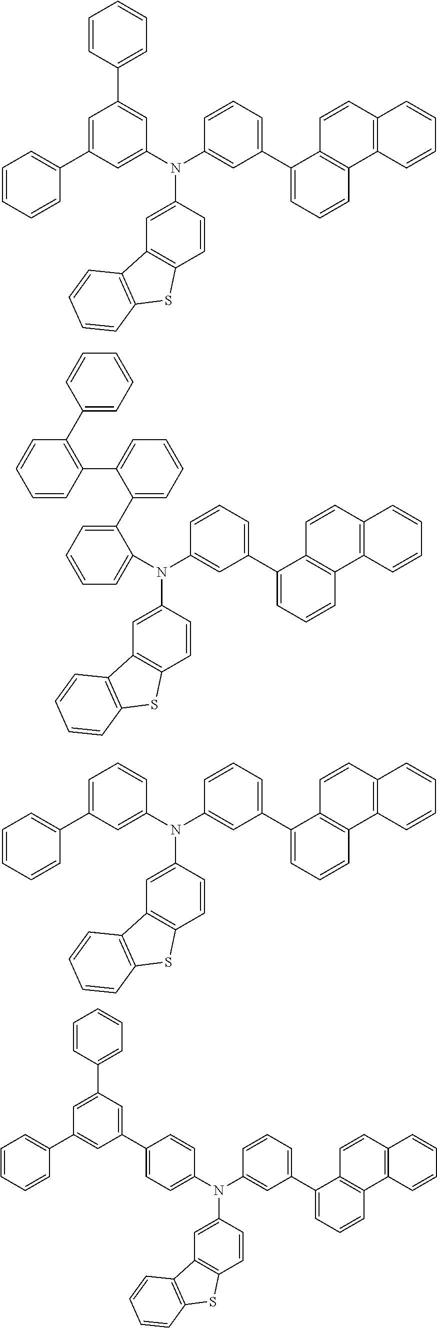

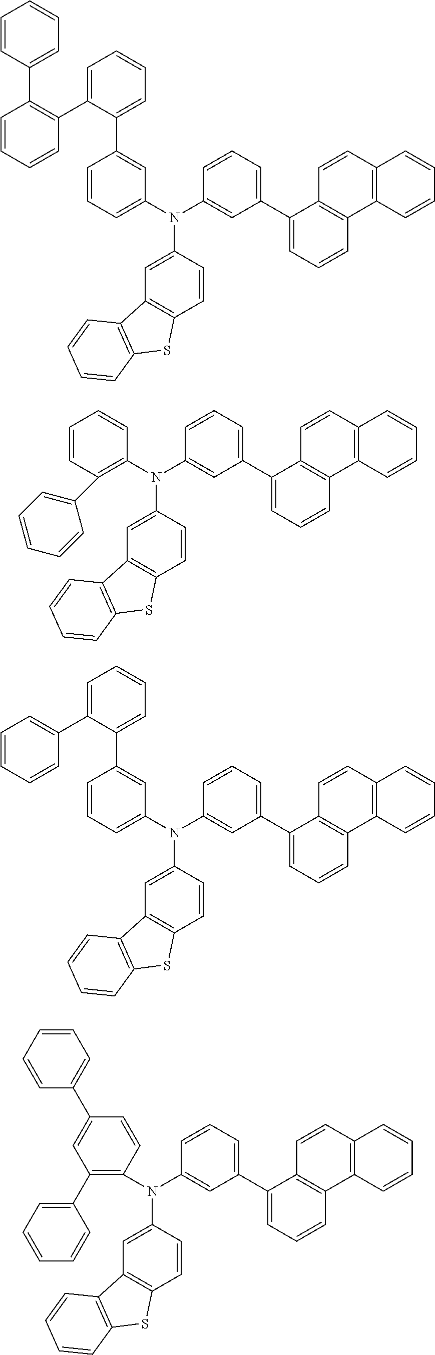

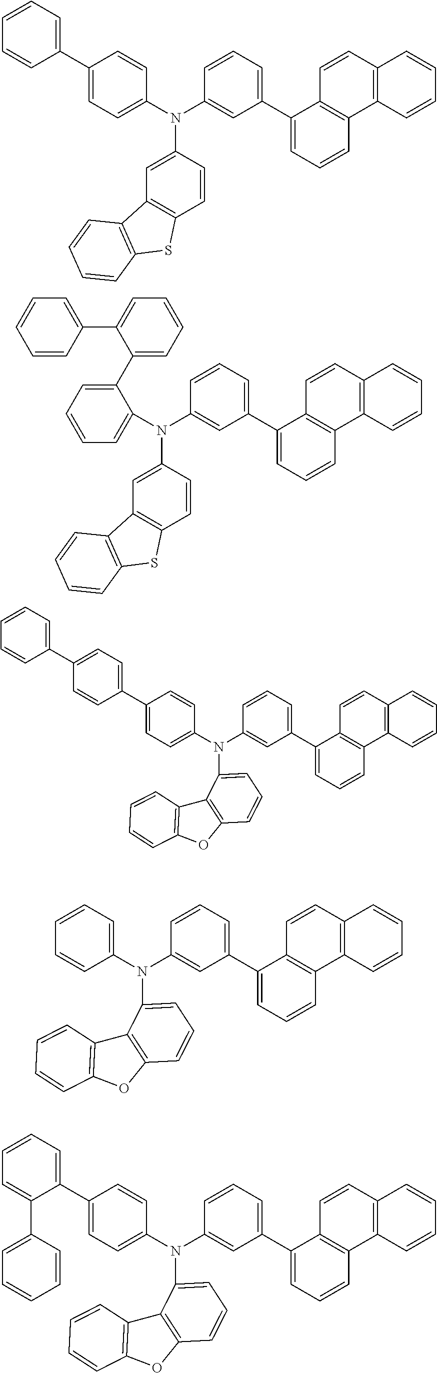

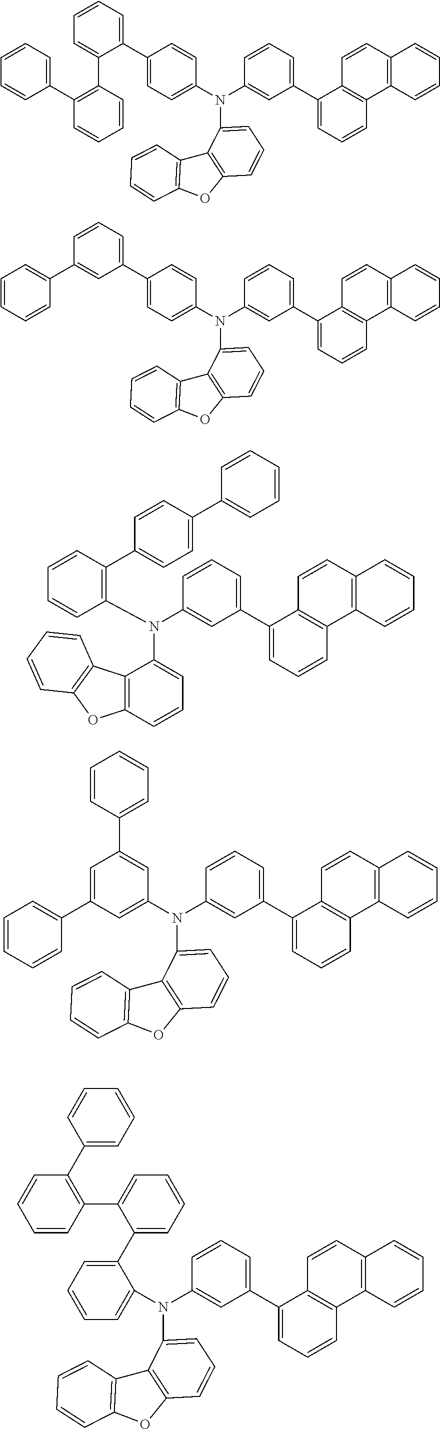

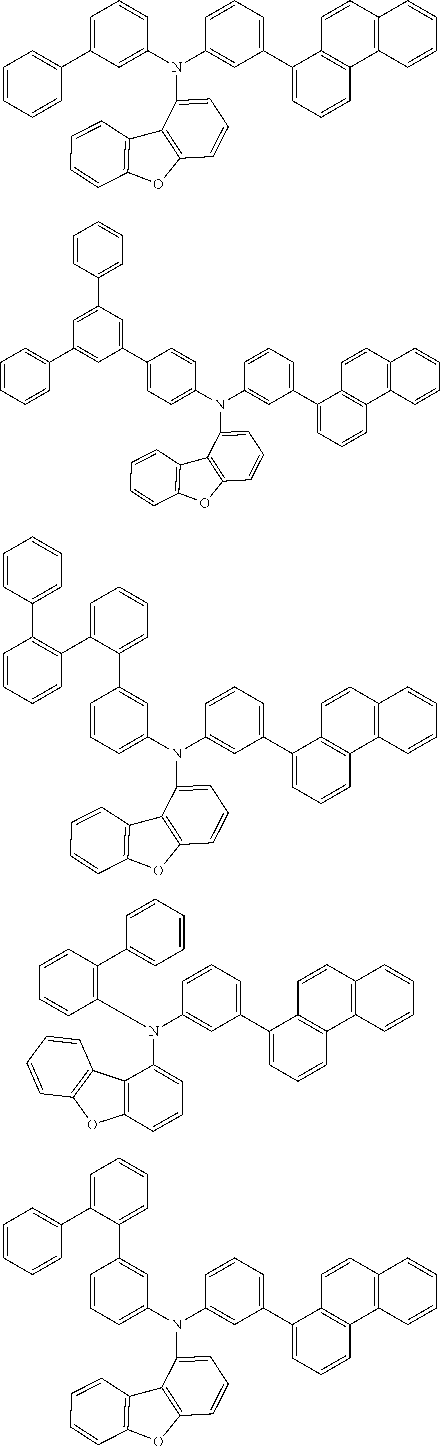

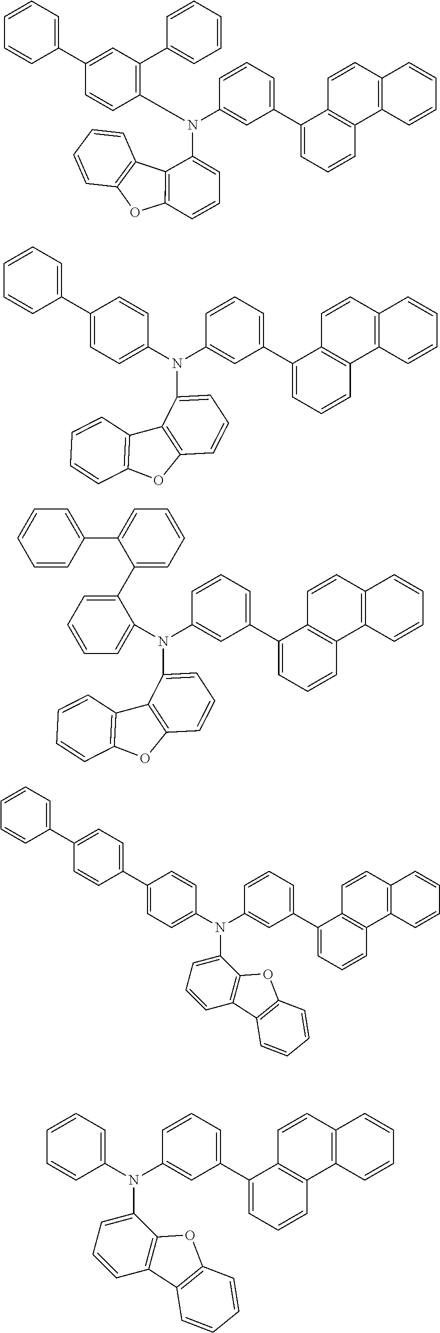

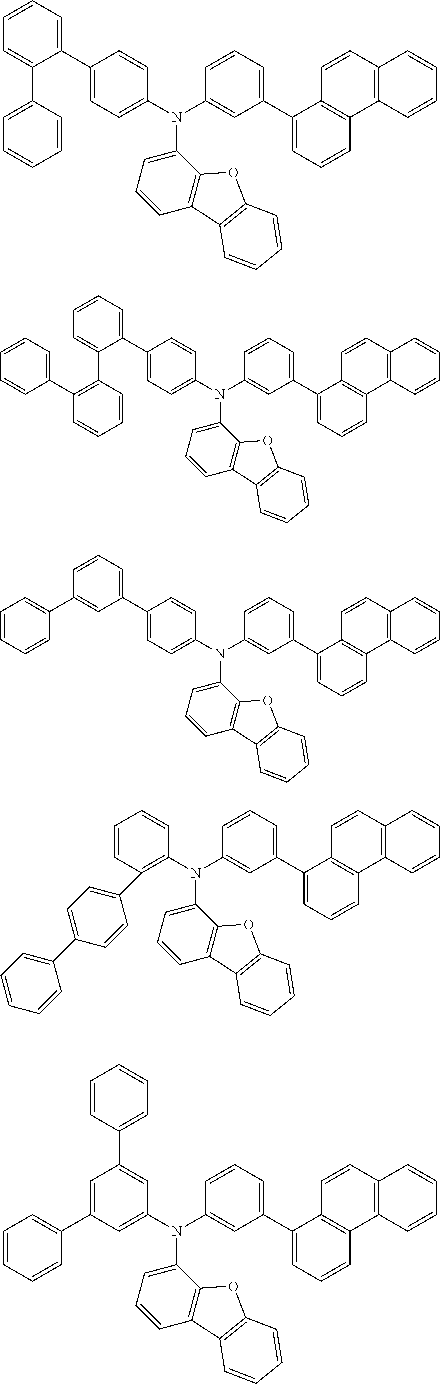

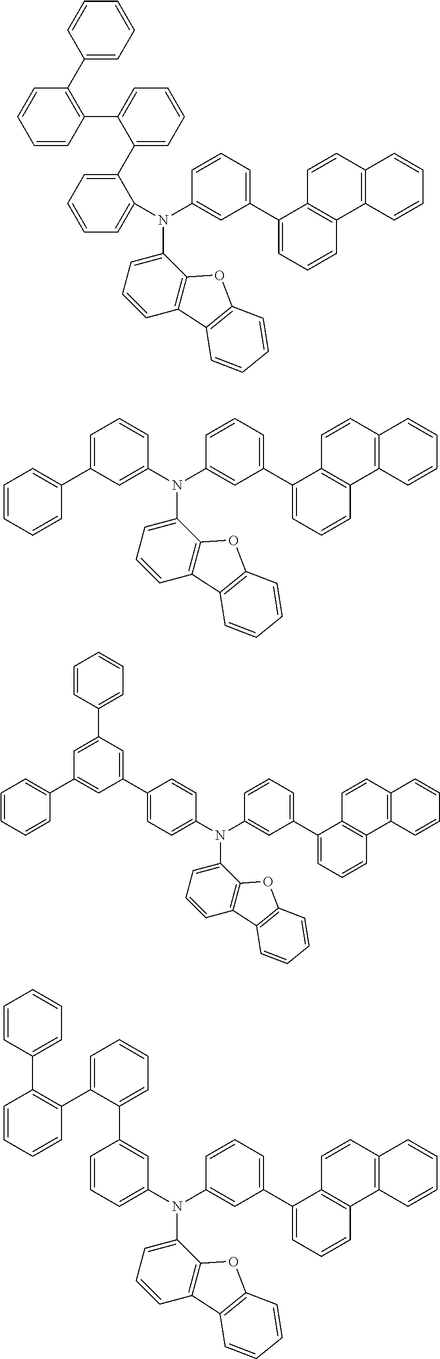 [SMILES: c1ccc(-c2ccccc2-c2ccccc2-c2ccccc2N(c2cccc(-c3cccc4c3ccc3ccccc34)c2)c2cccc3c2oc2ccccc23)cc1 ; c1ccc(-c2cccc(N(c3cccc(-c4cccc5c4ccc4ccccc45)c3)c3cccc4c3oc3ccccc34)c2)cc1 ; c1ccc(-c2cc(-c3ccccc3)cc(-c3ccc(N(c4cccc(-c5cccc6c5ccc5ccccc56)c4)c4cccc5c4oc4ccccc45)cc3)c2)cc1 ; c1ccc(-c2ccccc2-c2ccccc2-c2cccc(N(c3cccc(-c4cccc5c4ccc4ccccc45)c3)c3cccc4c3oc3ccccc34)c2)cc1]

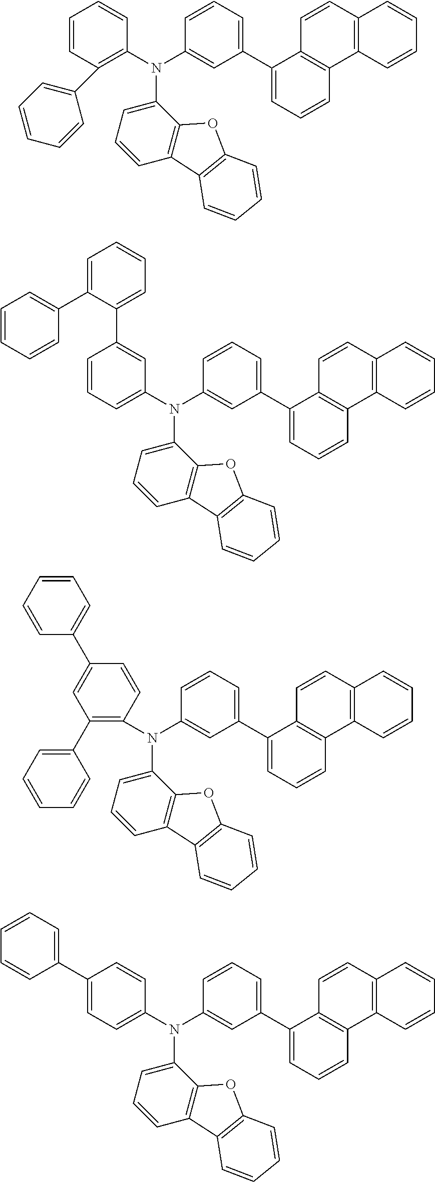 [SMILES: c1ccc(-c2ccccc2N(c2cccc(-c3cccc4c3ccc3ccccc34)c2)c2cccc3c2oc2ccccc23)cc1 ; c1ccc(-c2ccccc2-c2cccc(N(c3cccc(-c4cccc5c4ccc4ccccc45)c3)c3cccc4c3oc3ccccc34)c2)cc1 ; c1ccc(-c2ccc(N(c3cccc(-c4cccc5c4ccc4ccccc45)c3)c3cccc4c3oc3ccccc34)c(-c3ccccc3)c2)cc1 ; c1ccc(-c2ccc(N(c3cccc(-c4cccc5c4ccc4ccccc45)c3)c3cccc4c3oc3ccccc34)cc2)cc1]

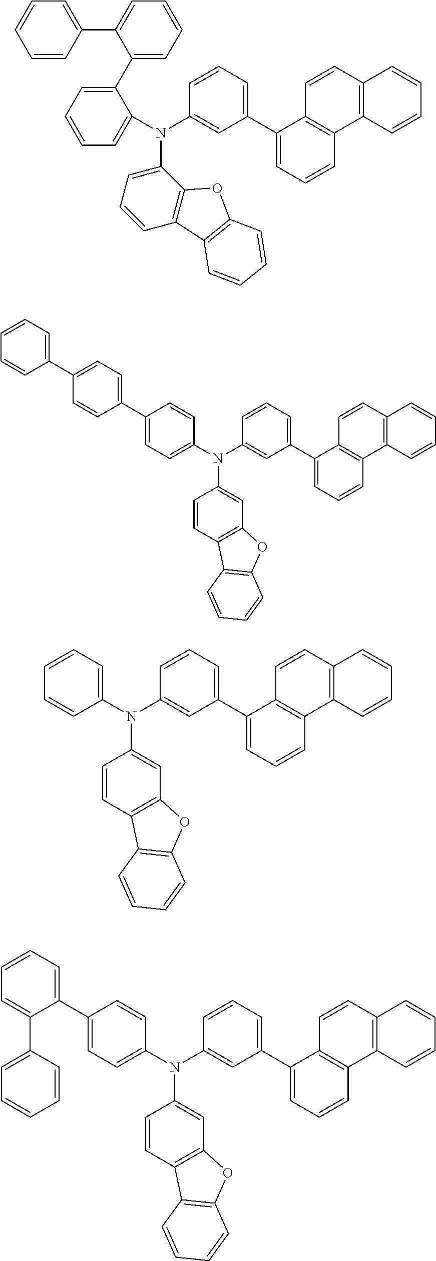

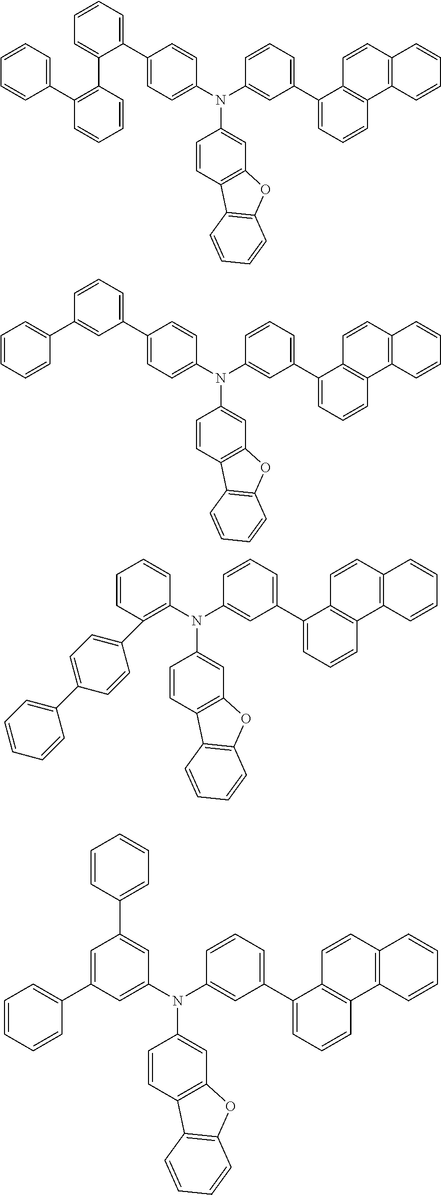

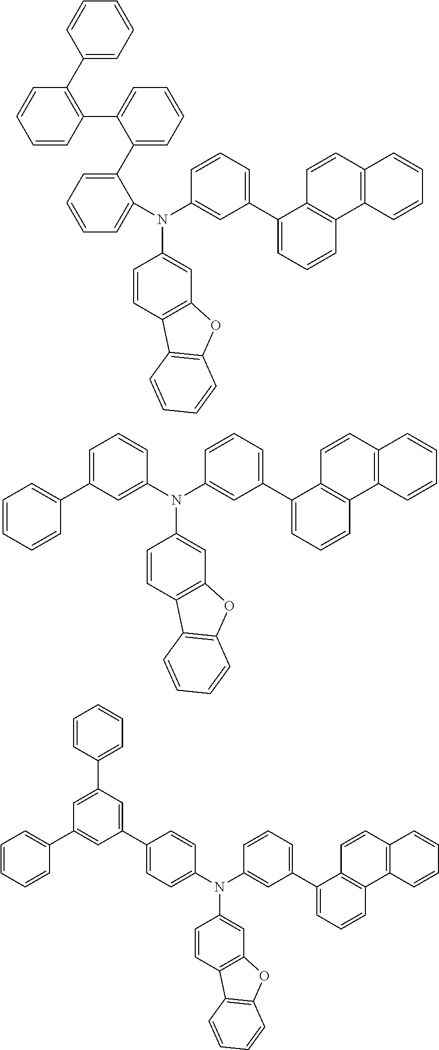

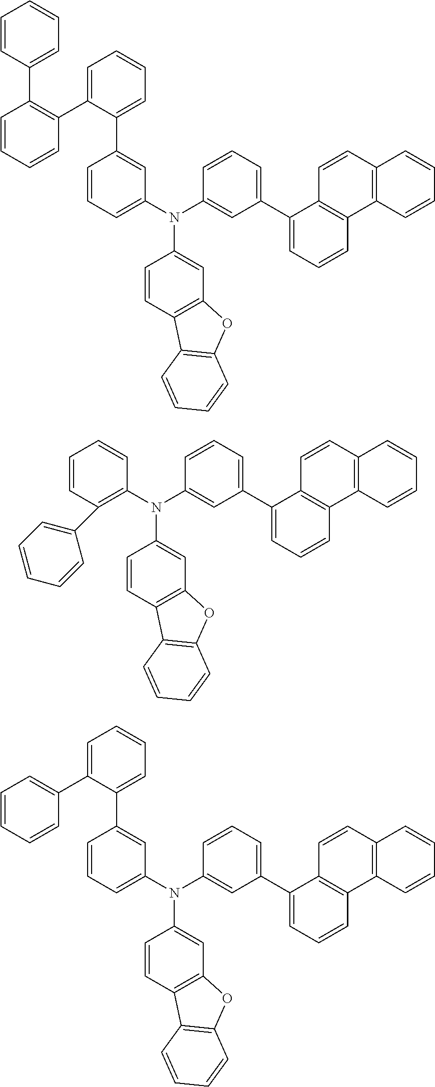

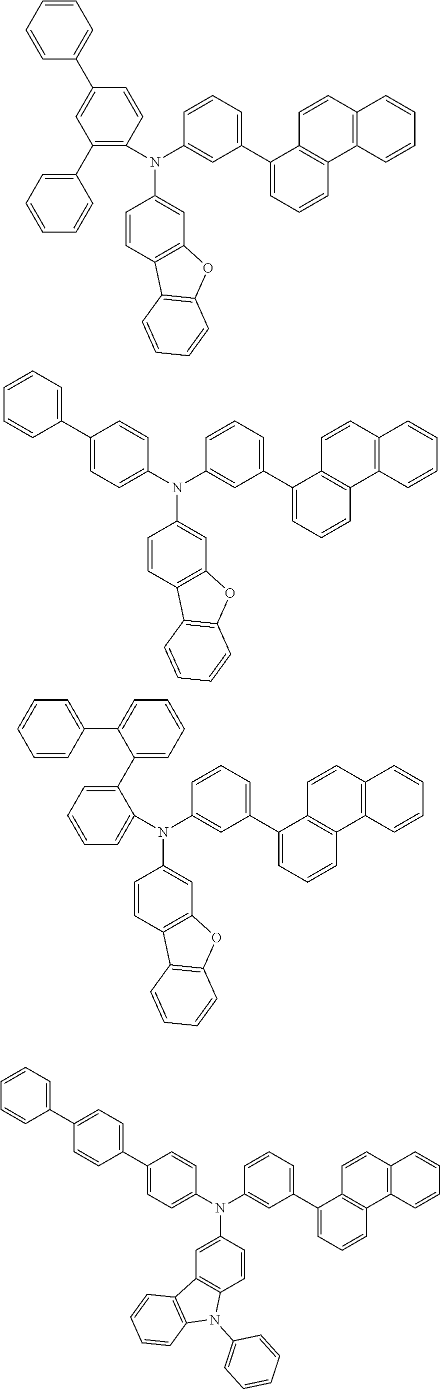

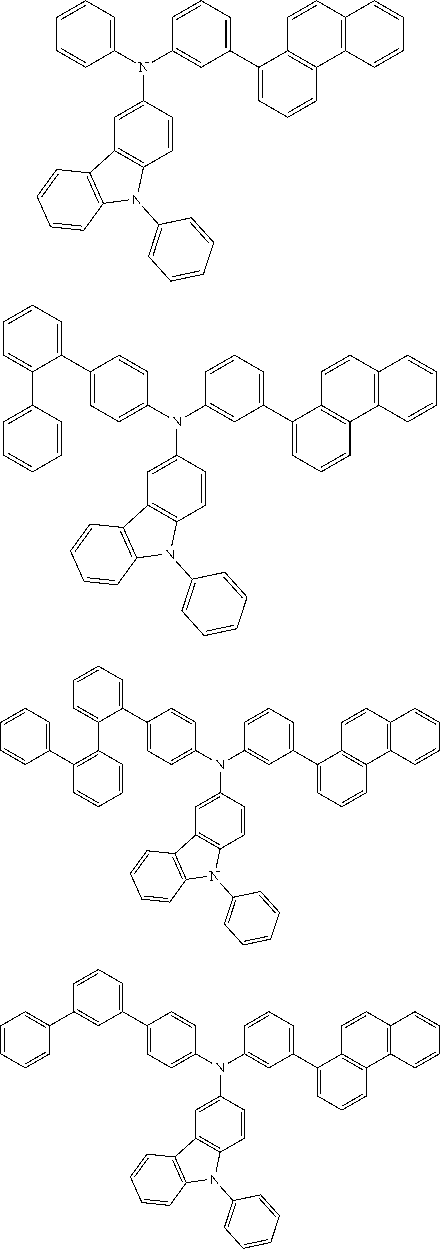 [SMILES: c1ccc(N(c2cccc(-c3cccc4c3ccc3ccccc34)c2)c2ccc3c(c2)c2ccccc2n3-c2ccccc2)cc1 ; c1ccc(-c2ccccc2-c2ccc(N(c3cccc(-c4cccc5c4ccc4ccccc45)c3)c3ccc4c(c3)c3ccccc3n4-c3ccccc3)cc2)cc1 ; c1ccc(-c2ccccc2-c2ccccc2-c2ccc(N(c3cccc(-c4cccc5c4ccc4ccccc45)c3)c3ccc4c(c3)c3ccccc3n4-c3ccccc3)cc2)cc1 ; c1ccc(-c2cccc(-c3ccc(N(c4cccc(-c5cccc6c5ccc5ccccc56)c4)c4ccc5c(c4)c4ccccc4n5-c4ccccc4)cc3)c2)cc1]

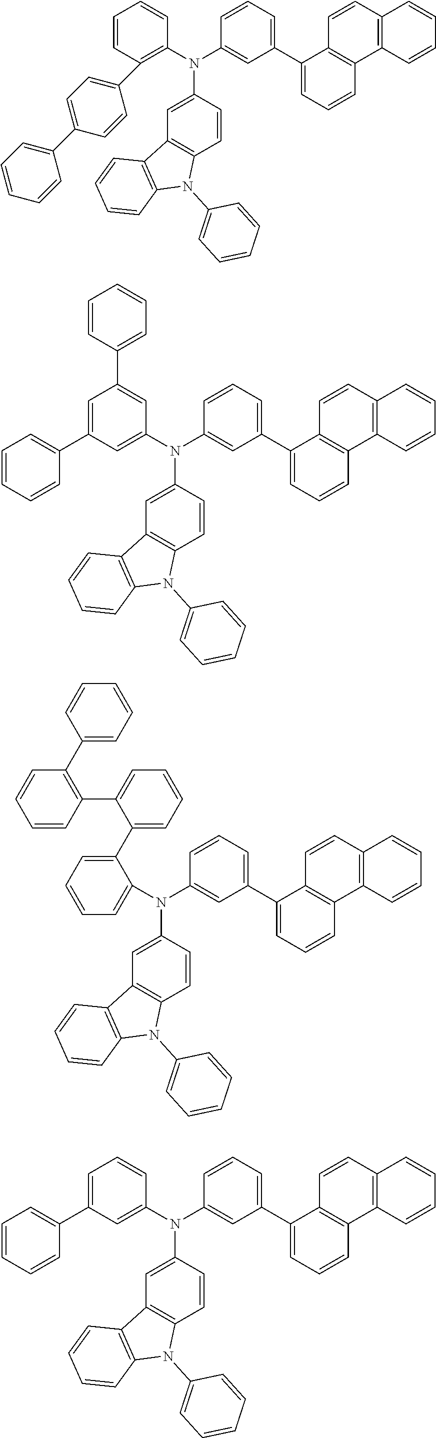 [SMILES: c1ccc(-c2ccc(-c3ccccc3N(c3cccc(-c4cccc5c4ccc4ccccc45)c3)c3ccc4c(c3)c3ccccc3n4-c3ccccc3)cc2)cc1 ; c1ccc(-c2cc(-c3ccccc3)cc(N(c3cccc(-c4cccc5c4ccc4ccccc45)c3)c3ccc4c(c3)c3ccccc3n4-c3ccccc3)c2)cc1 ; c1ccc(-c2ccccc2-c2ccccc2-c2ccccc2N(c2cccc(-c3cccc4c3ccc3ccccc34)c2)c2ccc3c(c2)c2ccccc2n3-c2ccccc2)cc1 ; c1ccc(-c2cccc(N(c3cccc(-c4cccc5c4ccc4ccccc45)c3)c3ccc4c(c3)c3ccccc3n4-c3ccccc3)c2)cc1]

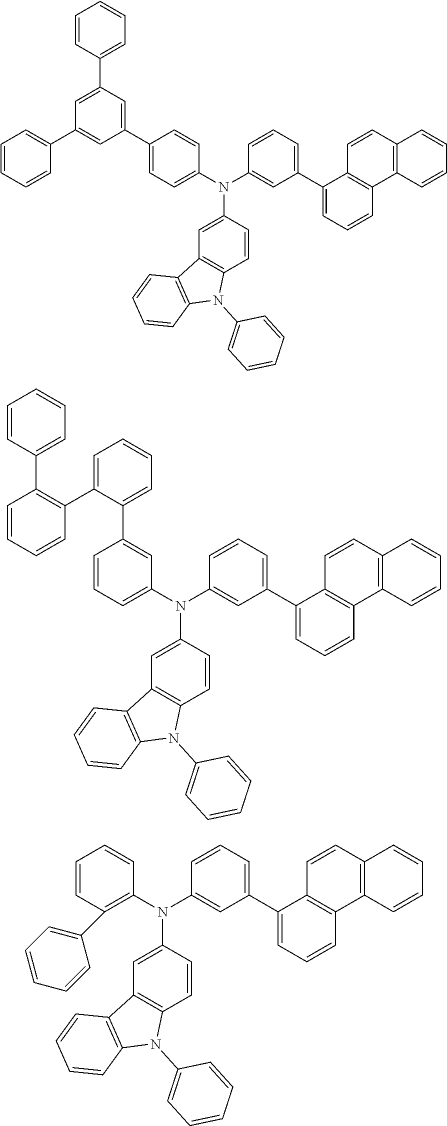

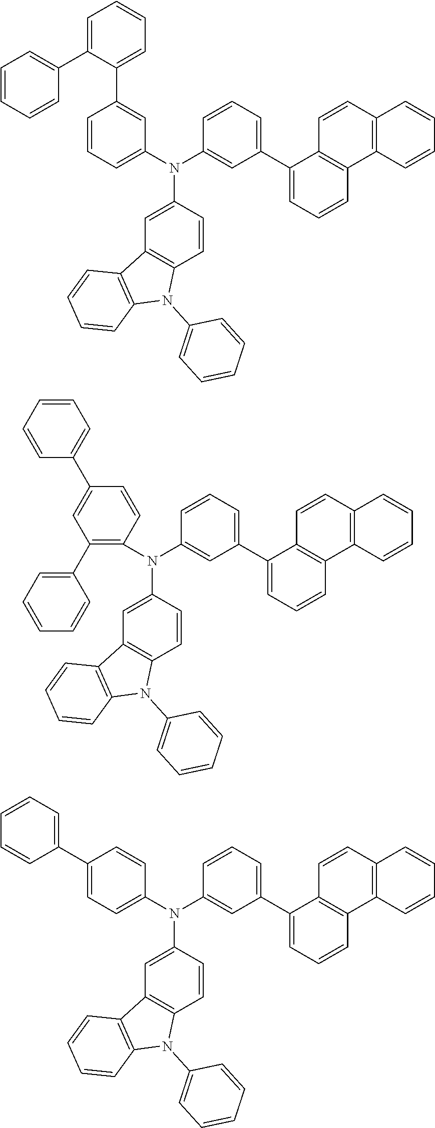 [SMILES: c1ccc(-c2ccccc2-c2cccc(N(c3cccc(-c4cccc5c4ccc4ccccc45)c3)c3ccc4c(c3)c3ccccc3n4-c3ccccc3)c2)cc1 ; c1ccc(-c2ccc(N(c3cccc(-c4cccc5c4ccc4ccccc45)c3)c3ccc4c(c3)c3ccccc3n4-c3ccccc3)c(-c3ccccc3)c2)cc1 ; c1ccc(-c2ccc(N(c3cccc(-c4cccc5c4ccc4ccccc45)c3)c3ccc4c(c3)c3ccccc3n4-c3ccccc3)cc2)cc1]

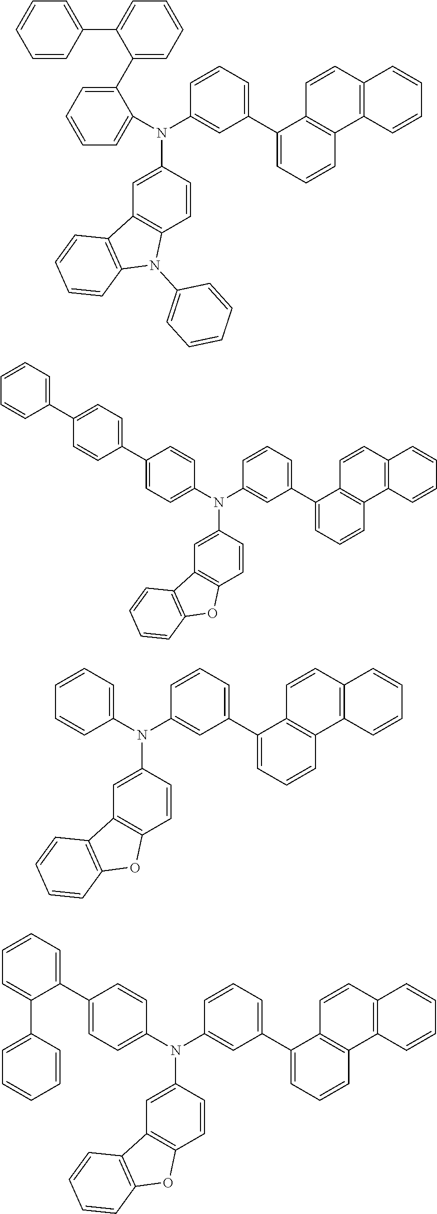

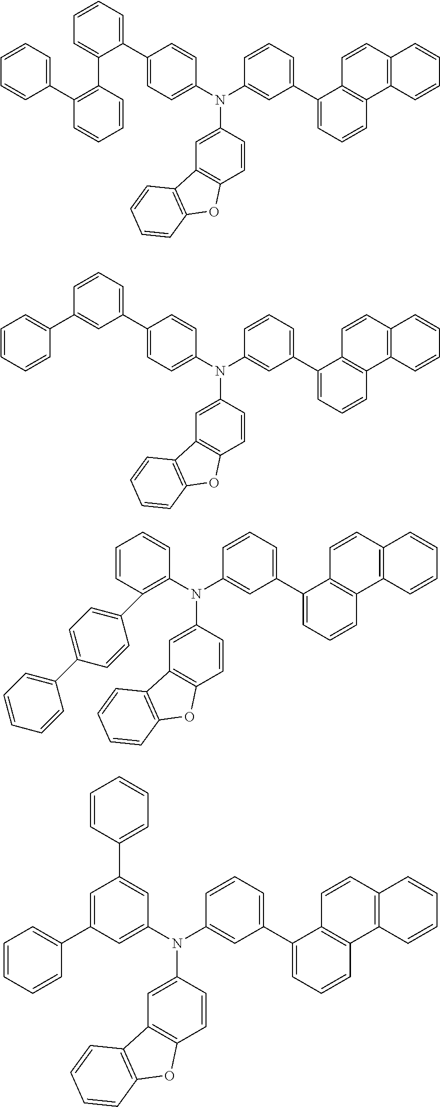

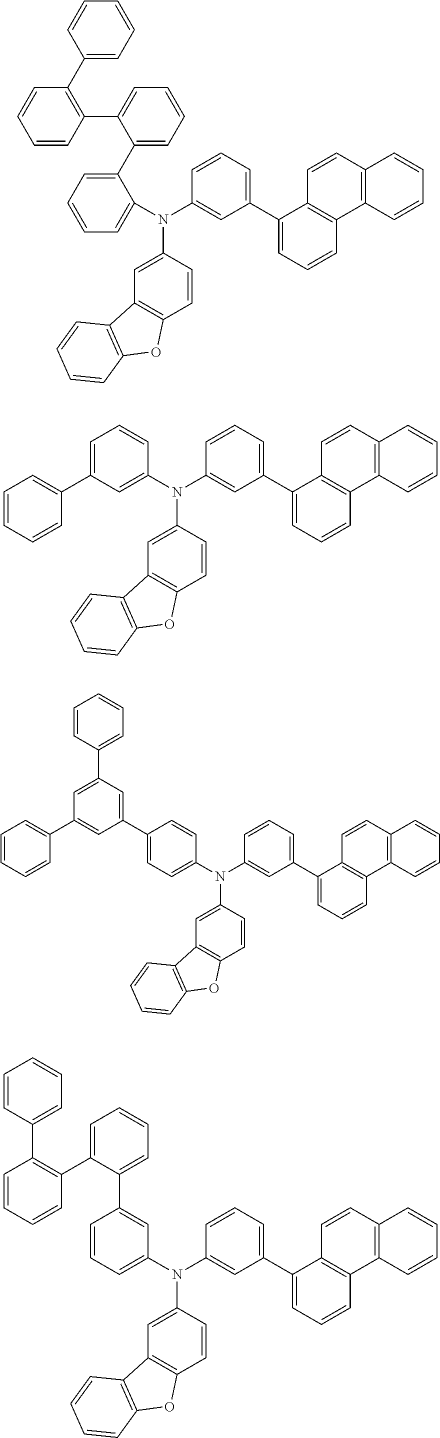

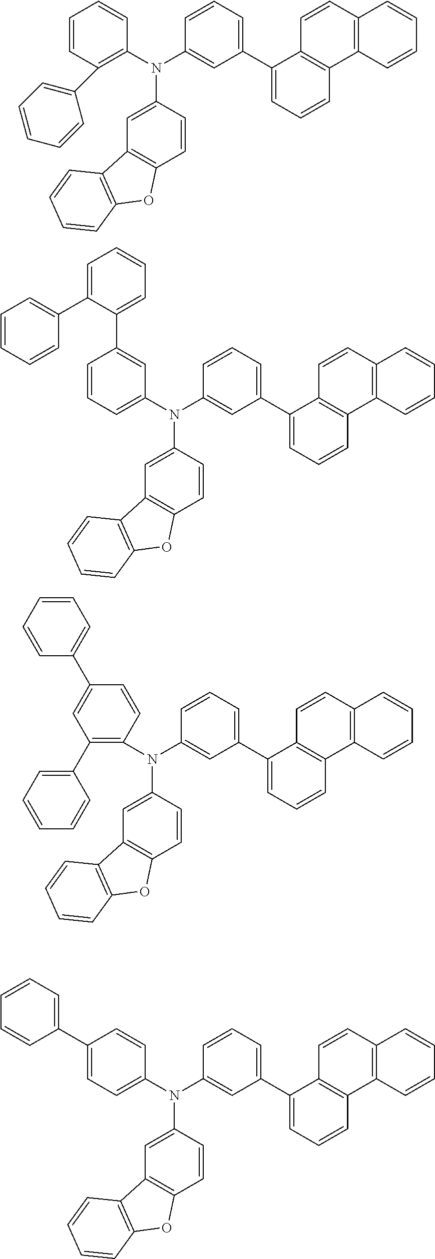

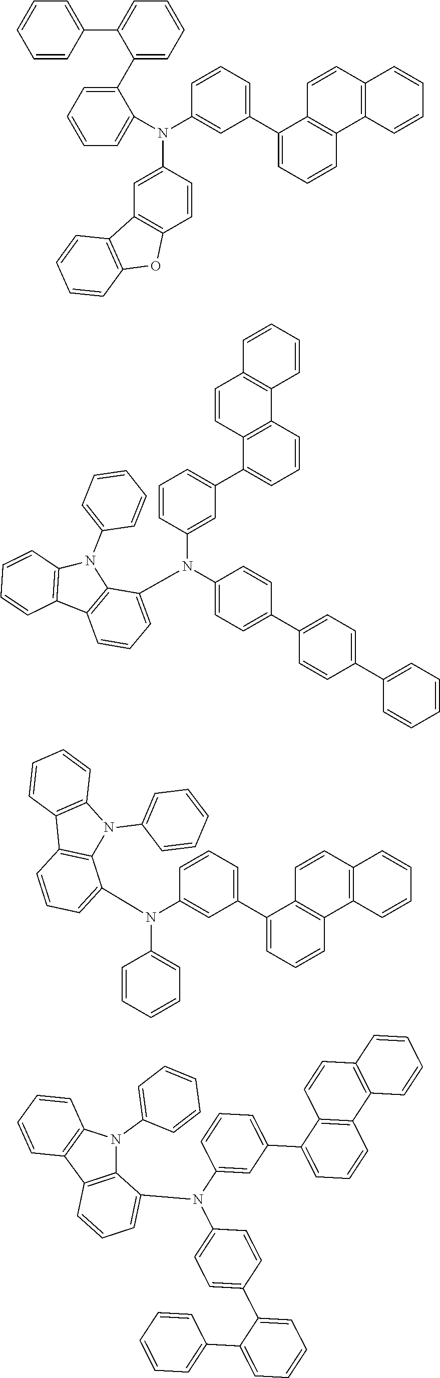 [SMILES: c1ccc(-c2ccccc2-c2ccccc2N(c2cccc(-c3cccc4c3ccc3ccccc34)c2)c2ccc3oc4ccccc4c3c2)cc1 ; c1ccc(-c2ccc(-c3ccc(N(c4cccc(-c5cccc6c5ccc5ccccc56)c4)c4cccc5c6ccccc6n(-c6ccccc6)c45)cc3)cc2)cc1 ; c1ccc(N(c2cccc(-c3cccc4c3ccc3ccccc34)c2)c2cccc3c4ccccc4n(-c4ccccc4)c23)cc1 ; c1ccc(-c2ccccc2-c2ccc(N(c3cccc(-c4cccc5c4ccc4ccccc45)c3)c3cccc4c5ccccc5n(-c5ccccc5)c34)cc2)cc1]

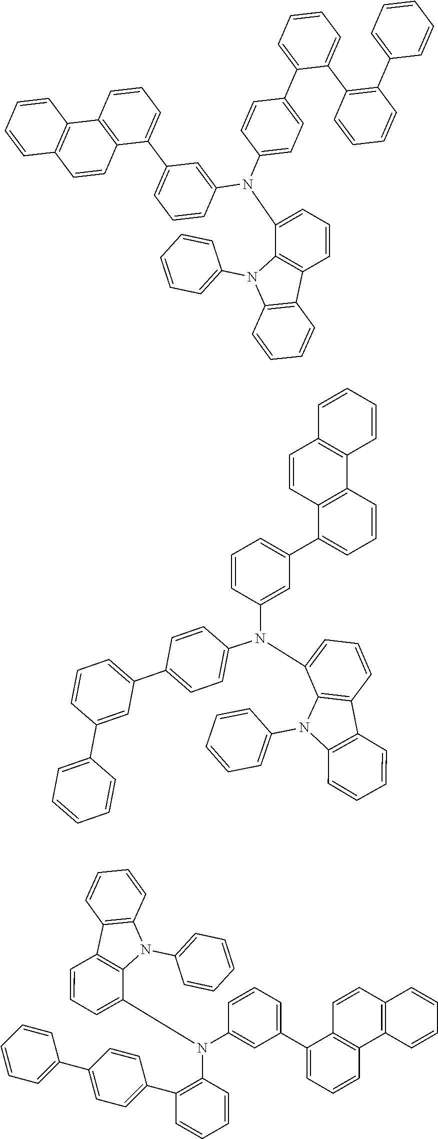 [SMILES: c1ccc(-c2ccccc2-c2ccccc2-c2ccc(N(c3cccc(-c4cccc5c4ccc4ccccc45)c3)c3cccc4c5ccccc5n(-c5ccccc5)c34)cc2)cc1 ; c1ccc(-c2cccc(-c3ccc(N(c4cccc(-c5cccc6c5ccc5ccccc56)c4)c4cccc5c6ccccc6n(-c6ccccc6)c45)cc3)c2)cc1 ; c1ccc(-c2ccc(-c3ccccc3N(c3cccc(-c4cccc5c4ccc4ccccc45)c3)c3cccc4c5ccccc5n(-c5ccccc5)c34)cc2)cc1]

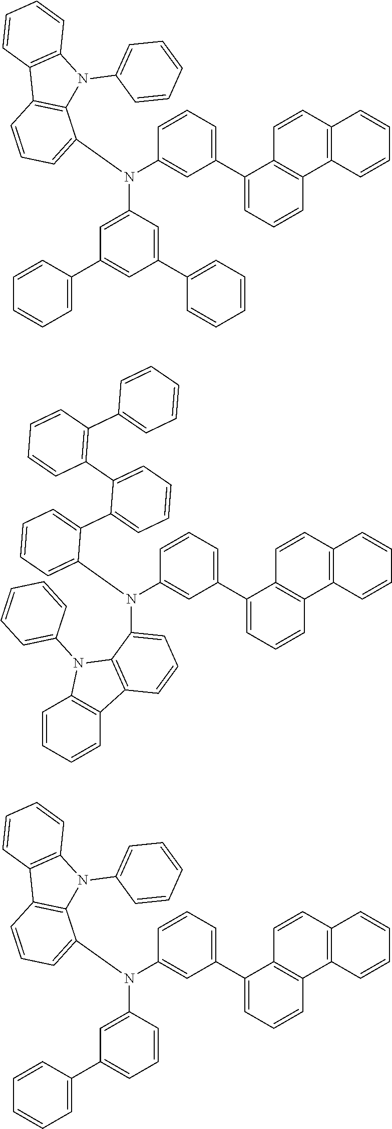 [SMILES: c1ccc(-c2cc(-c3ccccc3)cc(N(c3cccc(-c4cccc5c4ccc4ccccc45)c3)c3cccc4c5ccccc5n(-c5ccccc5)c34)c2)cc1 ; c1ccc(-c2ccccc2-c2ccccc2-c2ccccc2N(c2cccc(-c3cccc4c3ccc3ccccc34)c2)c2cccc3c4ccccc4n(-c4ccccc4)c23)cc1 ; c1ccc(-c2cccc(N(c3cccc(-c4cccc5c4ccc4ccccc45)c3)c3cccc4c5ccccc5n(-c5ccccc5)c34)c2)cc1]

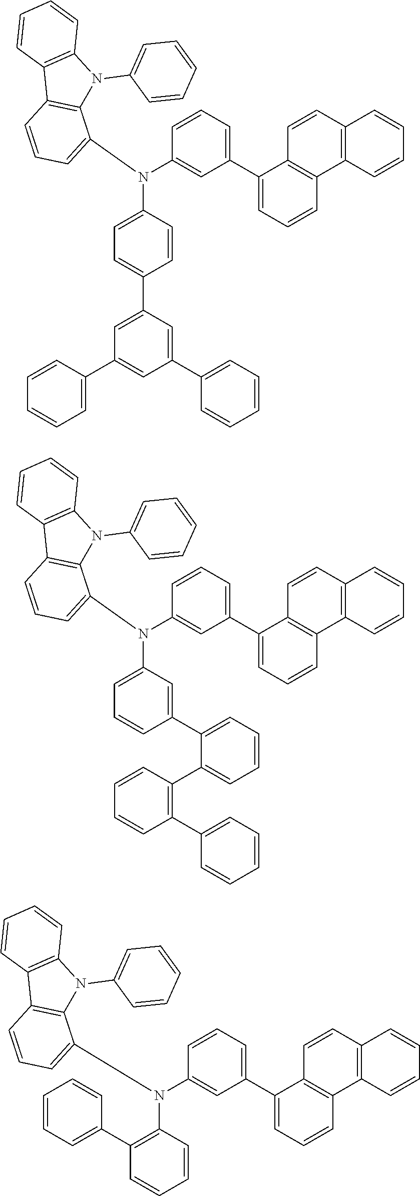

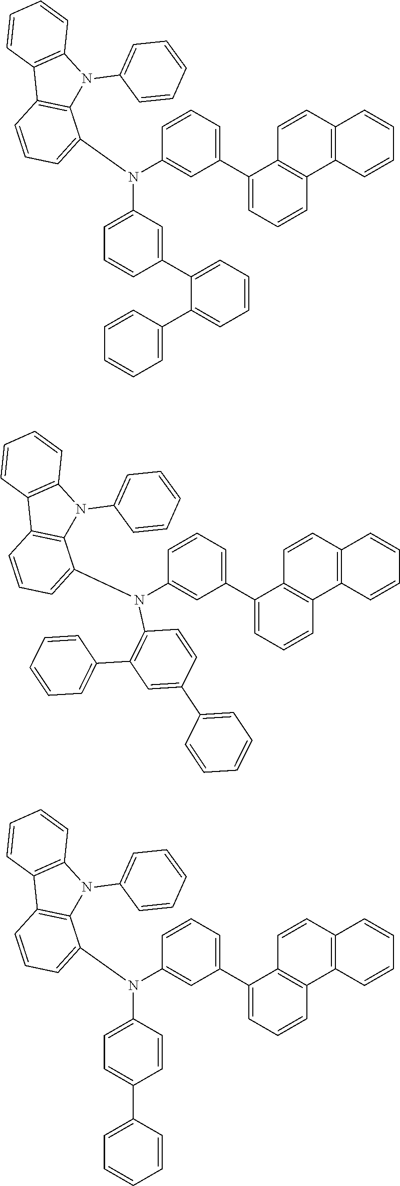 [SMILES: c1ccc(-c2ccccc2-c2cccc(N(c3cccc(-c4cccc5c4ccc4ccccc45)c3)c3cccc4c5ccccc5n(-c5ccccc5)c34)c2)cc1 ; c1ccc(-c2ccc(N(c3cccc(-c4cccc5c4ccc4ccccc45)c3)c3cccc4c5ccccc5n(-c5ccccc5)c34)c(-c3ccccc3)c2)cc1 ; c1ccc(-c2ccc(N(c3cccc(-c4cccc5c4ccc4ccccc45)c3)c3cccc4c5ccccc5n(-c5ccccc5)c34)cc2)cc1]

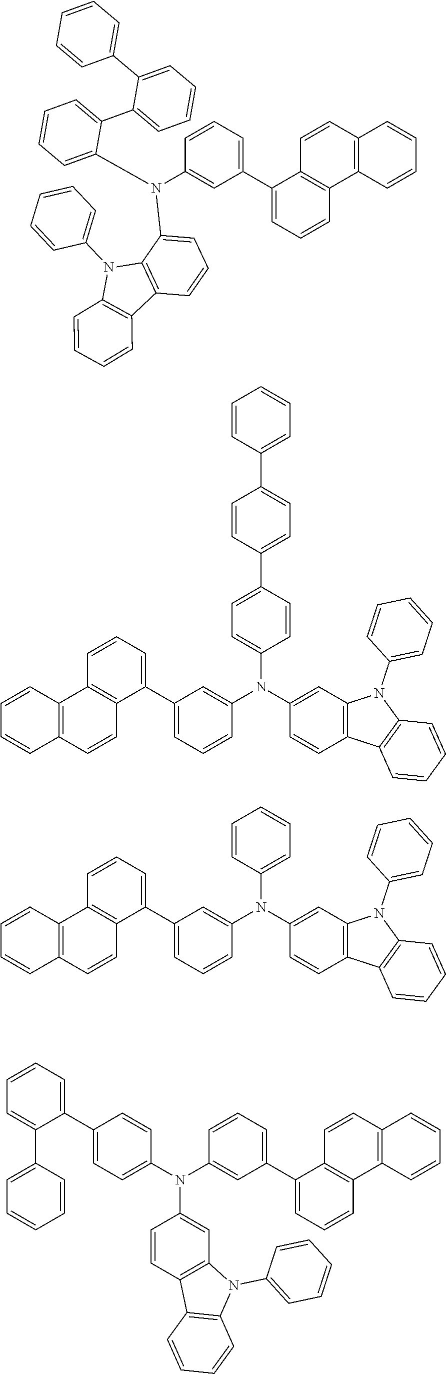

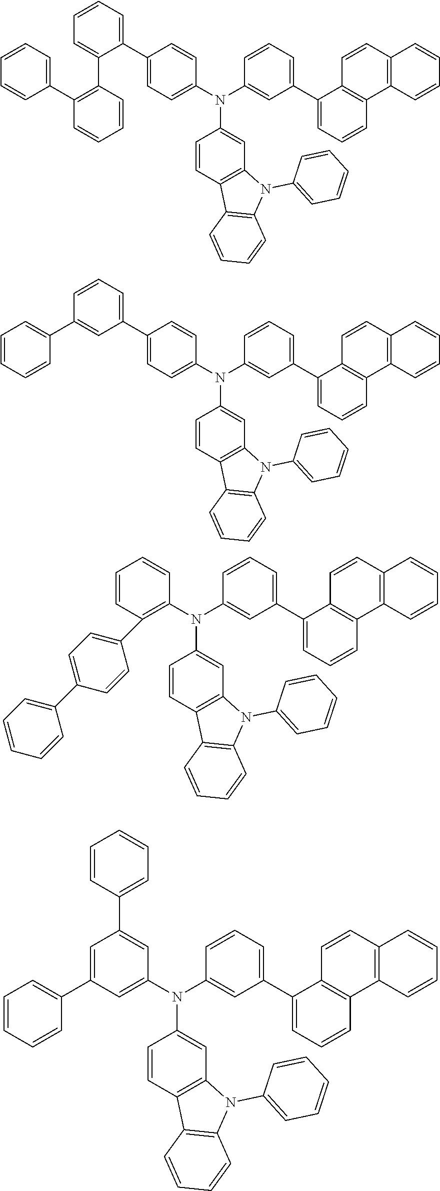

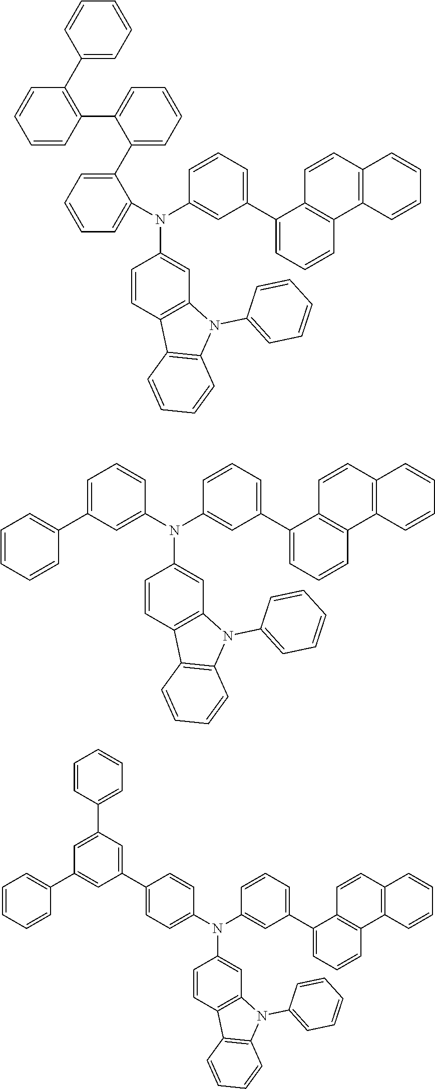 [SMILES: c1ccc(-c2ccccc2-c2ccccc2-c2ccccc2N(c2cccc(-c3cccc4c3ccc3ccccc34)c2)c2ccc3c4ccccc4n(-c4ccccc4)c3c2)cc1 ; c1ccc(-c2cccc(N(c3cccc(-c4cccc5c4ccc4ccccc45)c3)c3ccc4c5ccccc5n(-c5ccccc5)c4c3)c2)cc1 ; c1ccc(-c2cc(-c3ccccc3)cc(-c3ccc(N(c4cccc(-c5cccc6c5ccc5ccccc56)c4)c4ccc5c6ccccc6n(-c6ccccc6)c5c4)cc3)c2)cc1]

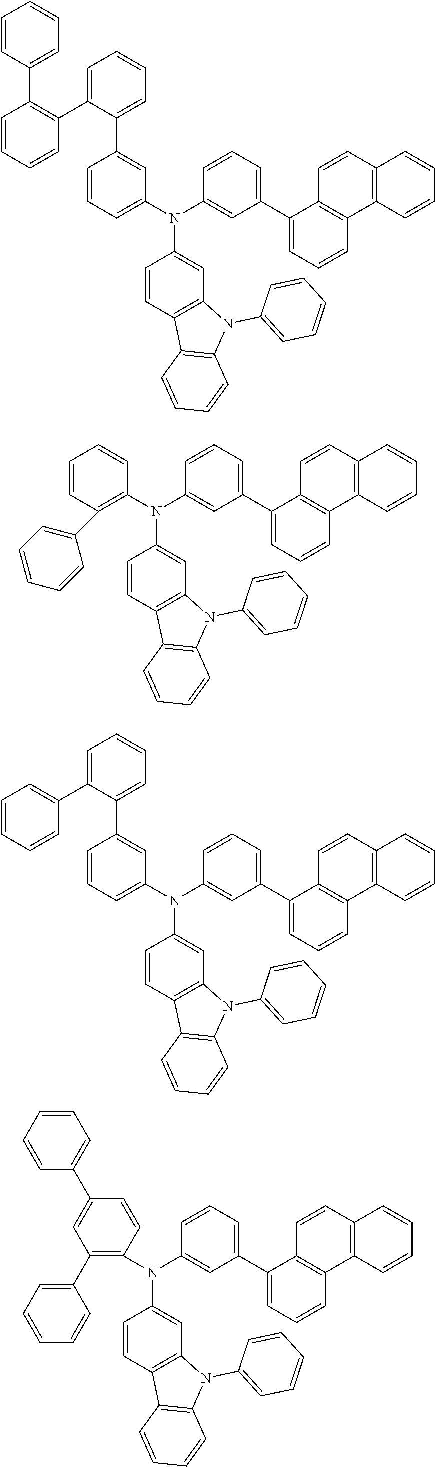

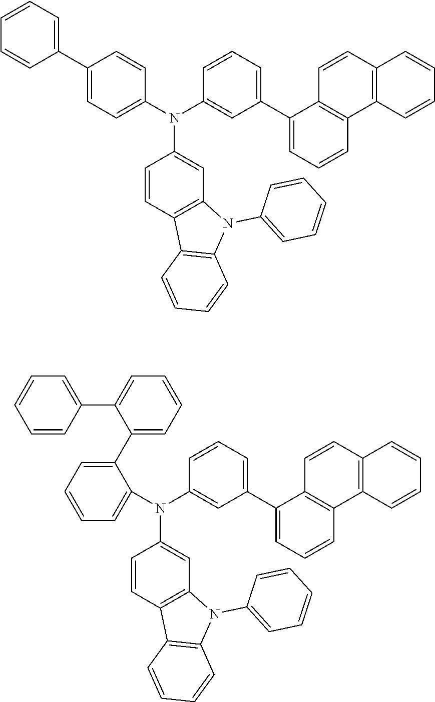 [SMILES: c1ccc(-c2ccc(N(c3cccc(-c4cccc5c4ccc4ccccc45)c3)c3ccc4c5ccccc5n(-c5ccccc5)c4c3)cc2)cc1 ; c1ccc(-c2ccccc2-c2ccccc2N(c2cccc(-c3cccc4c3ccc3ccccc34)c2)c2ccc3c4ccccc4n(-c4ccccc4)c3c2)cc1]

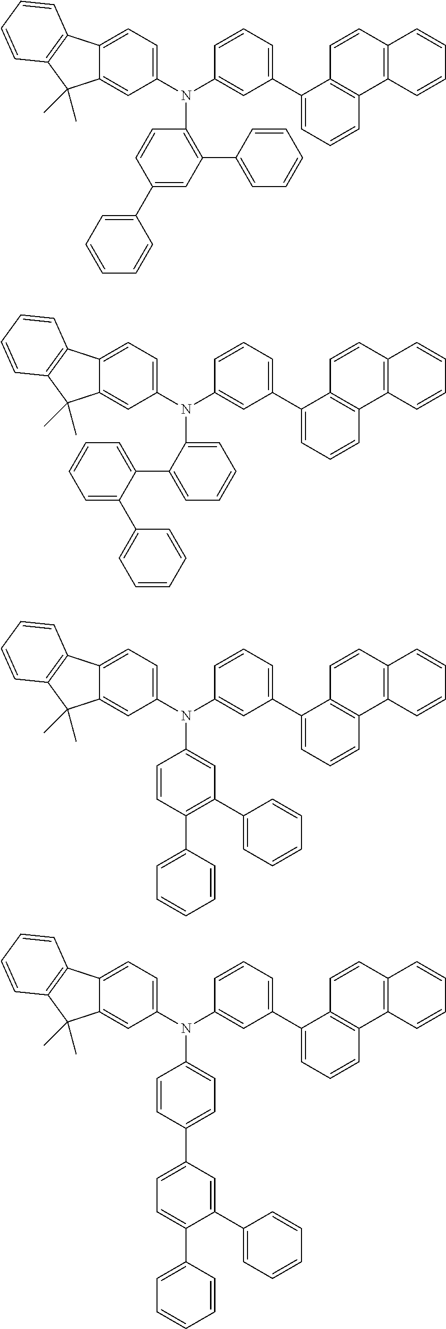 [SMILES: CC1(C)c2ccccc2-c2ccc(N(c3cccc(-c4cccc5c4ccc4ccccc45)c3)c3ccc(-c4ccccc4)cc3-c3ccccc3)cc21 ; CC1(C)c2ccccc2-c2ccc(N(c3cccc(-c4cccc5c4ccc4ccccc45)c3)c3ccccc3-c3ccccc3-c3ccccc3)cc21 ; CC1(C)c2ccccc2-c2ccc(N(c3cccc(-c4cccc5c4ccc4ccccc45)c3)c3ccc(-c4ccccc4)c(-c4ccccc4)c3)cc21 ; CC1(C)c2ccccc2-c2ccc(N(c3ccc(-c4ccc(-c5ccccc5)c(-c5ccccc5)c4)cc3)c3cccc(-c4cccc5c4ccc4ccccc45)c3)cc21]

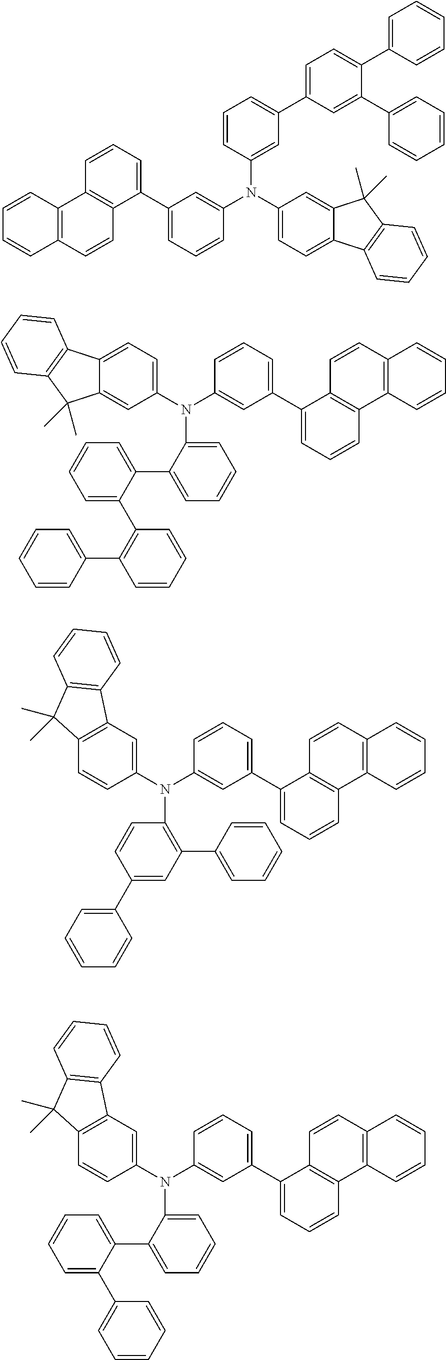

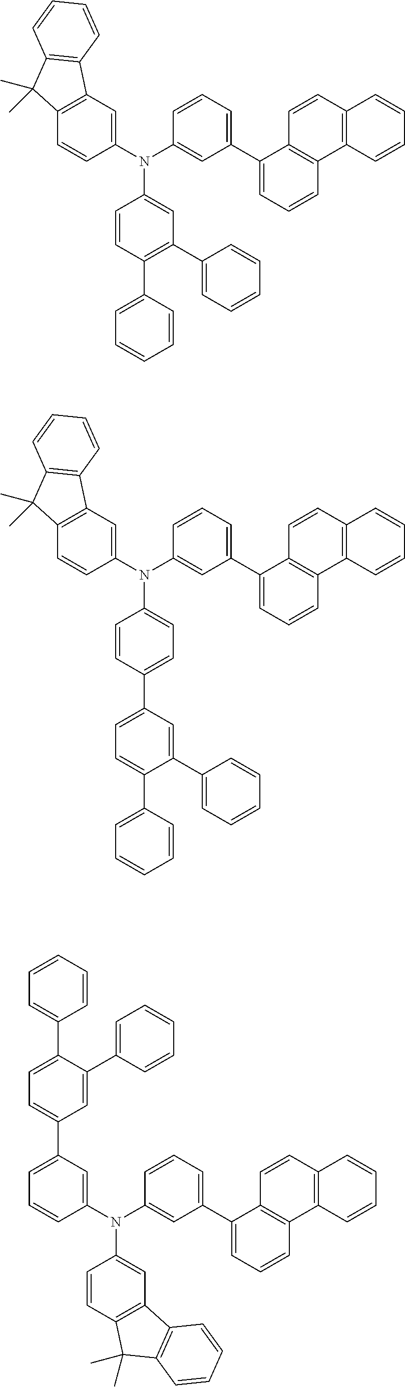 [SMILES: CC1(C)c2ccccc2-c2cc(N(c3cccc(-c4cccc5c4ccc4ccccc45)c3)c3ccc(-c4ccccc4)c(-c4ccccc4)c3)ccc21 ; CC1(C)c2ccccc2-c2cc(N(c3ccc(-c4ccc(-c5ccccc5)c(-c5ccccc5)c4)cc3)c3cccc(-c4cccc5c4ccc4ccccc45)c3)ccc21 ; CC1(C)c2ccccc2-c2cc(N(c3cccc(-c4ccc(-c5ccccc5)c(-c5ccccc5)c4)c3)c3cccc(-c4cccc5c4ccc4ccccc45)c3)ccc21]

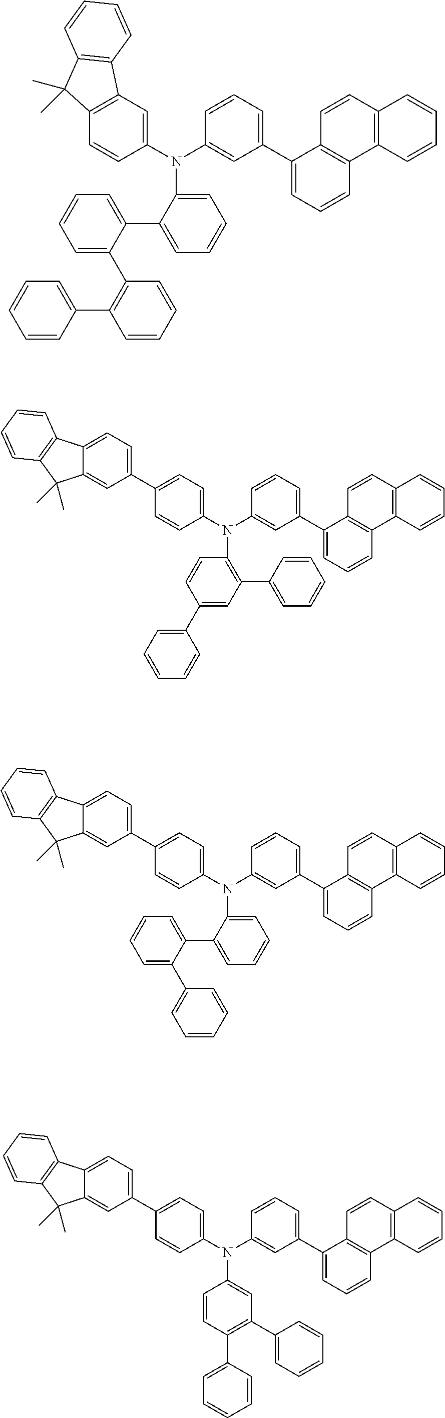 [SMILES: CC1(C)c2ccccc2-c2cc(N(c3cccc(-c4cccc5c4ccc4ccccc45)c3)c3ccccc3-c3ccccc3-c3ccccc3-c3ccccc3)ccc21 ; CC1(C)c2ccccc2-c2ccc(-c3ccc(N(c4cccc(-c5cccc6c5ccc5ccccc56)c4)c4ccc(-c5ccccc5)cc4-c4ccccc4)cc3)cc21 ; CC1(C)c2ccccc2-c2ccc(-c3ccc(N(c4cccc(-c5cccc6c5ccc5ccccc56)c4)c4ccccc4-c4ccccc4-c4ccccc4)cc3)cc21 ; CC1(C)c2ccccc2-c2ccc(-c3ccc(N(c4cccc(-c5cccc6c5ccc5ccccc56)c4)c4ccc(-c5ccccc5)c(-c5ccccc5)c4)cc3)cc21]

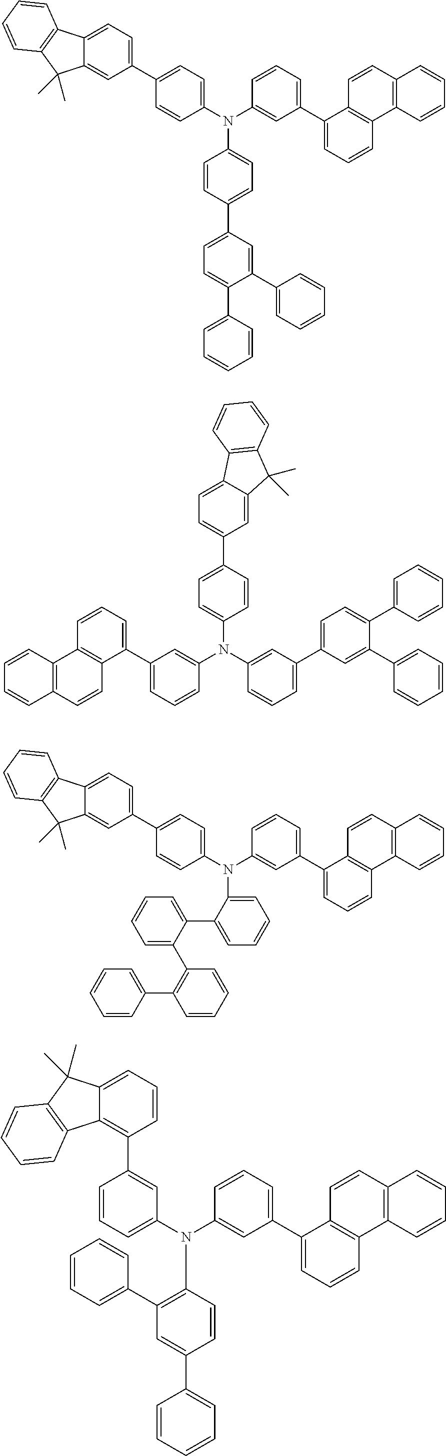

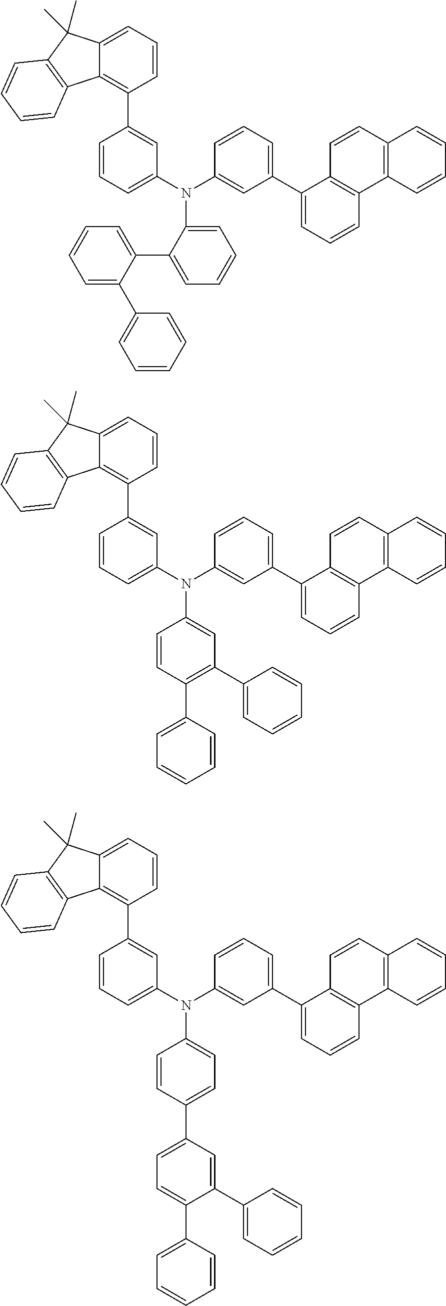 [SMILES: CC1(C)c2ccccc2-c2c(-c3cccc(N(c4cccc(-c5cccc6c5ccc5ccccc56)c4)c4ccccc4-c4ccccc4-c4ccccc4)c3)cccc21 ; CC1(C)c2ccccc2-c2c(-c3cccc(N(c4cccc(-c5cccc6c5ccc5ccccc56)c4)c4ccc(-c5ccccc5)c(-c5ccccc5)c4)c3)cccc21 ; CC1(C)c2ccccc2-c2c(-c3cccc(N(c4ccc(-c5ccc(-c6ccccc6)c(-c6ccccc6)c5)cc4)c4cccc(-c5cccc6c5ccc5ccccc56)c4)c3)cccc21]

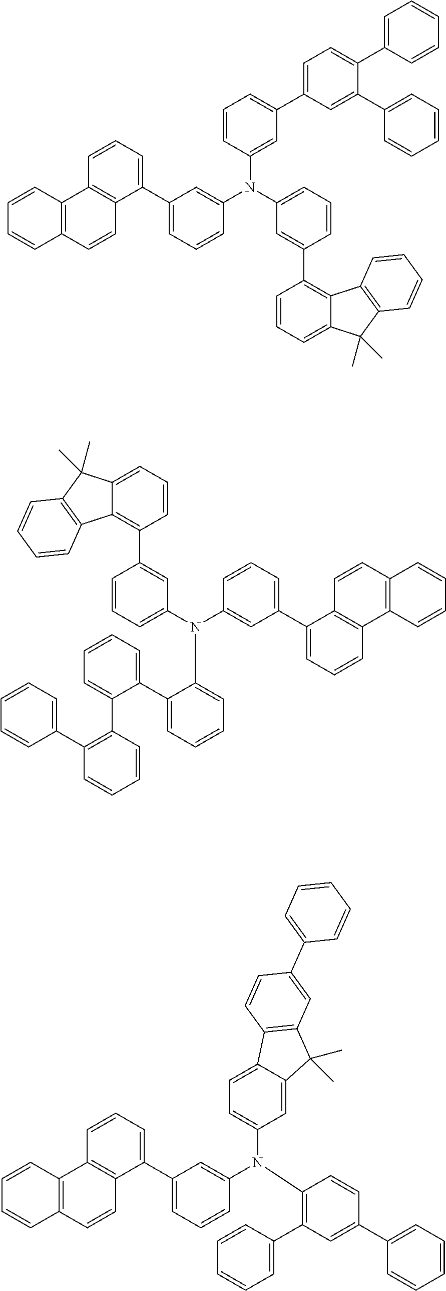 [SMILES: CC1(C)c2ccccc2-c2c(-c3cccc(N(c4cccc(-c5ccc(-c6ccccc6)c(-c6ccccc6)c5)c4)c4cccc(-c5cccc6c5ccc5ccccc56)c4)c3)cccc21 ; CC1(C)c2ccccc2-c2c(-c3cccc(N(c4cccc(-c5cccc6c5ccc5ccccc56)c4)c4ccccc4-c4ccccc4-c4ccccc4-c4ccccc4)c3)cccc21 ; CC1(C)c2cc(-c3ccccc3)ccc2-c2ccc(N(c3cccc(-c4cccc5c4ccc4ccccc45)c3)c3ccc(-c4ccccc4)cc3-c3ccccc3)cc21]

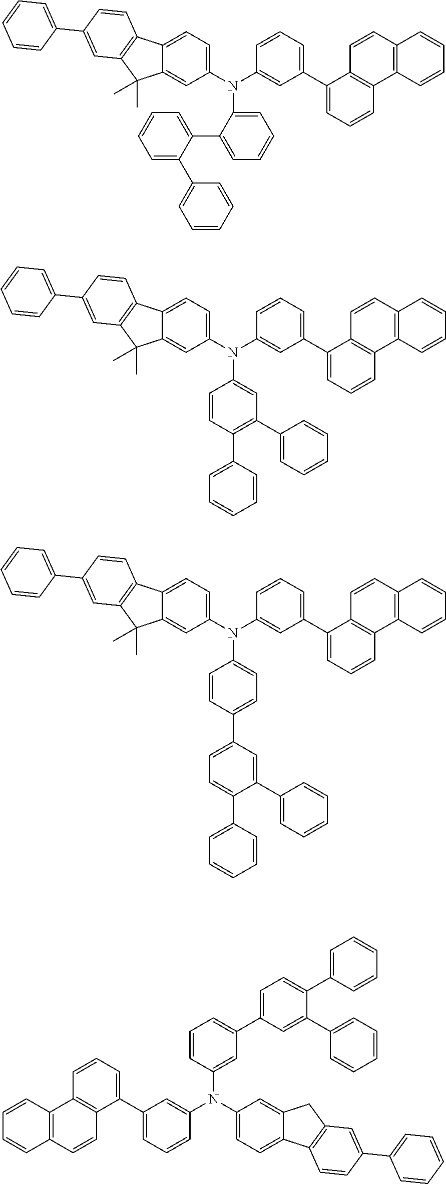 [SMILES: CC1(C)c2cc(-c3ccccc3)ccc2-c2ccc(N(c3cccc(-c4cccc5c4ccc4ccccc45)c3)c3ccccc3-c3ccccc3-c3ccccc3)cc21 ; CC1(C)c2cc(-c3ccccc3)ccc2-c2ccc(N(c3cccc(-c4cccc5c4ccc4ccccc45)c3)c3ccc(-c4ccccc4)c(-c4ccccc4)c3)cc21 ; CC1(C)c2cc(-c3ccccc3)ccc2-c2ccc(N(c3ccc(-c4ccc(-c5ccccc5)c(-c5ccccc5)c4)cc3)c3cccc(-c4cccc5c4ccc4ccccc45)c3)cc21 ; c1ccc(-c2ccc3c(c2)Cc2cc(N(c4cccc(-c5ccc(-c6ccccc6)c(-c6ccccc6)c5)c4)c4cccc(-c5cccc6c5ccc5ccccc56)c4)ccc2-3)cc1]

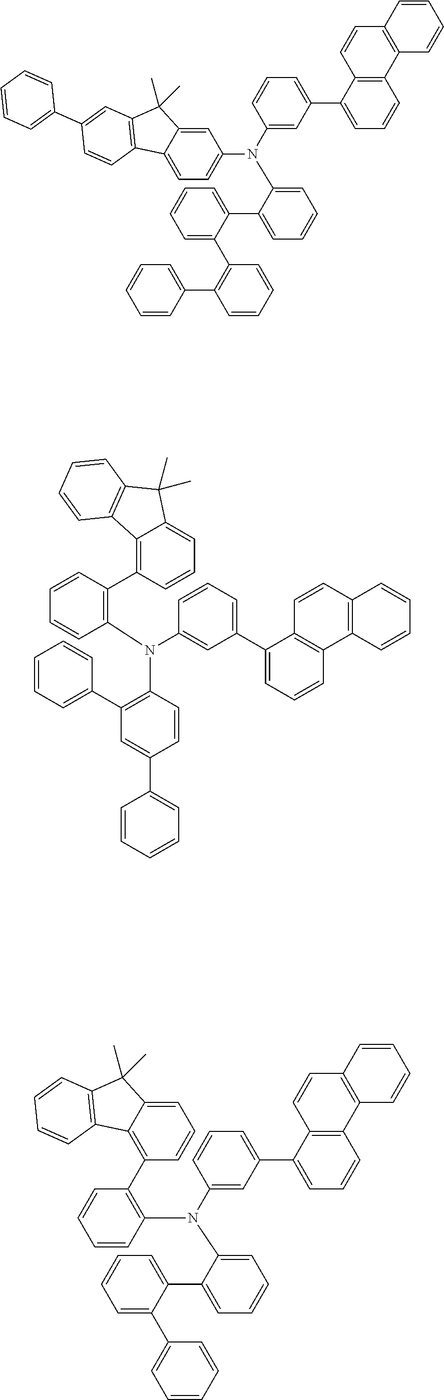 [SMILES: CC1(C)c2cc(-c3ccccc3)ccc2-c2ccc(N(c3cccc(-c4cccc5c4ccc4ccccc45)c3)c3ccccc3-c3ccccc3-c3ccccc3-c3ccccc3)cc21 ; CC1(C)c2ccccc2-c2c(-c3ccccc3N(c3cccc(-c4cccc5c4ccc4ccccc45)c3)c3ccc(-c4ccccc4)cc3-c3ccccc3)cccc21 ; CC1(C)c2ccccc2-c2c(-c3ccccc3N(c3cccc(-c4cccc5c4ccc4ccccc45)c3)c3ccccc3-c3ccccc3-c3ccccc3)cccc21]

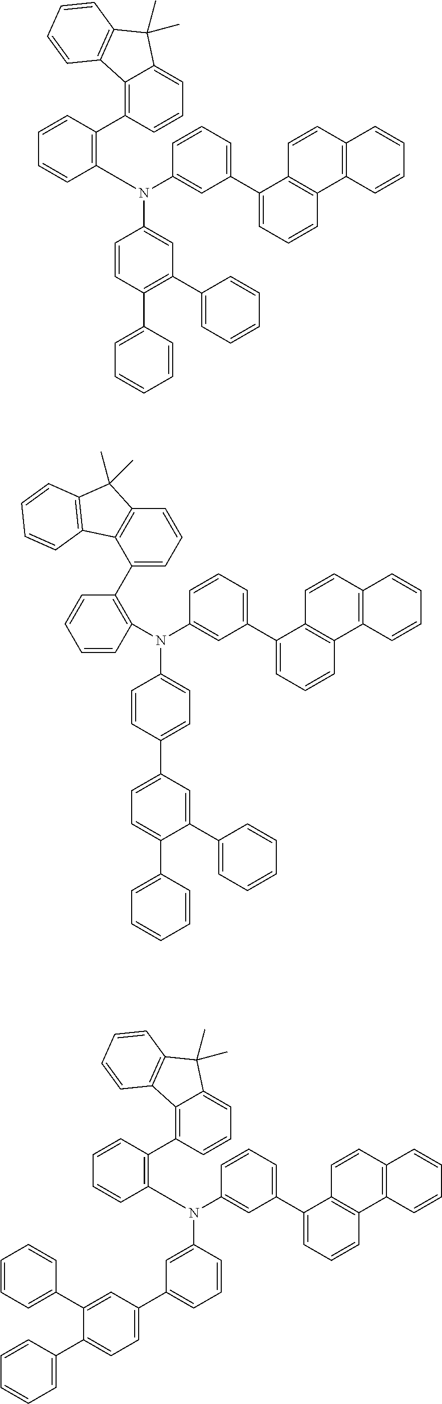 [SMILES: CC1(C)c2ccccc2-c2c(-c3ccccc3N(c3cccc(-c4cccc5c4ccc4ccccc45)c3)c3ccc(-c4ccccc4)c(-c4ccccc4)c3)cccc21 ; CC1(C)c2ccccc2-c2c(-c3ccccc3N(c3ccc(-c4ccc(-c5ccccc5)c(-c5ccccc5)c4)cc3)c3cccc(-c4cccc5c4ccc4ccccc45)c3)cccc21 ; CC1(C)c2ccccc2-c2c(-c3ccccc3N(c3cccc(-c4ccc(-c5ccccc5)c(-c5ccccc5)c4)c3)c3cccc(-c4cccc5c4ccc4ccccc45)c3)cccc21]

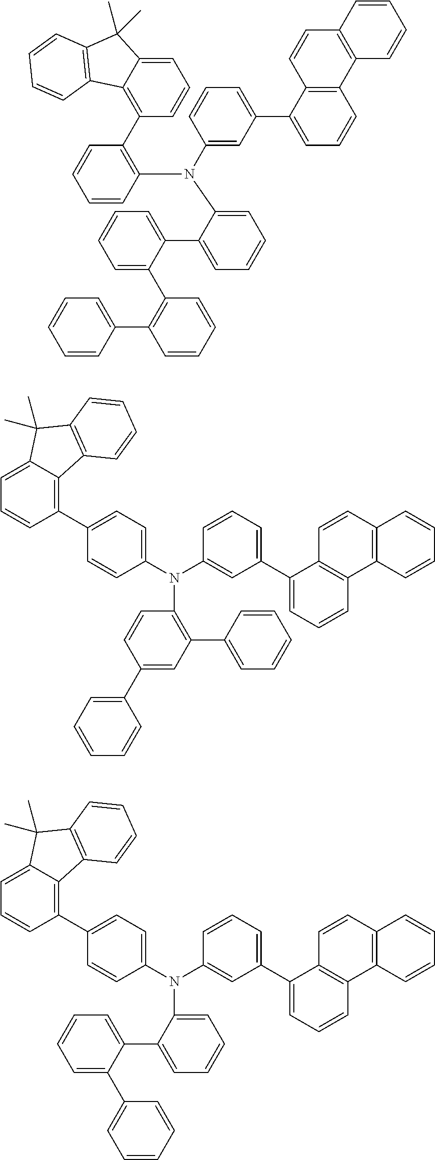

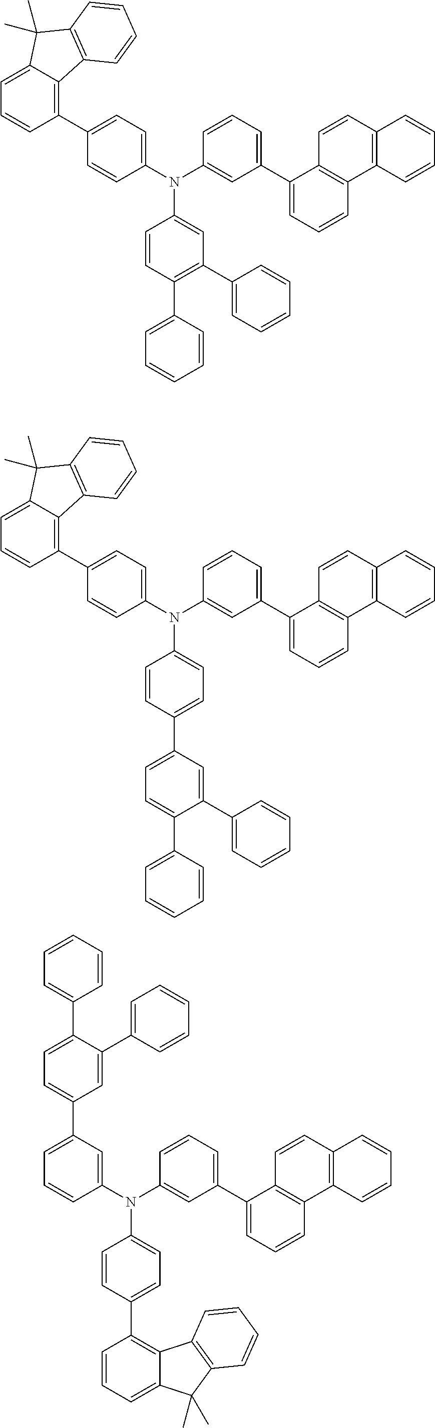 [SMILES: CC1(C)c2ccccc2-c2c(-c3ccc(N(c4cccc(-c5cccc6c5ccc5ccccc56)c4)c4ccc(-c5ccccc5)c(-c5ccccc5)c4)cc3)cccc21 ; CC1(C)c2ccccc2-c2c(-c3ccc(N(c4ccc(-c5ccc(-c6ccccc6)c(-c6ccccc6)c5)cc4)c4cccc(-c5cccc6c5ccc5ccccc56)c4)cc3)cccc21 ; CC1(C)c2ccccc2-c2c(-c3ccc(N(c4cccc(-c5ccc(-c6ccccc6)c(-c6ccccc6)c5)c4)c4cccc(-c5cccc6c5ccc5ccccc56)c4)cc3)cccc21]

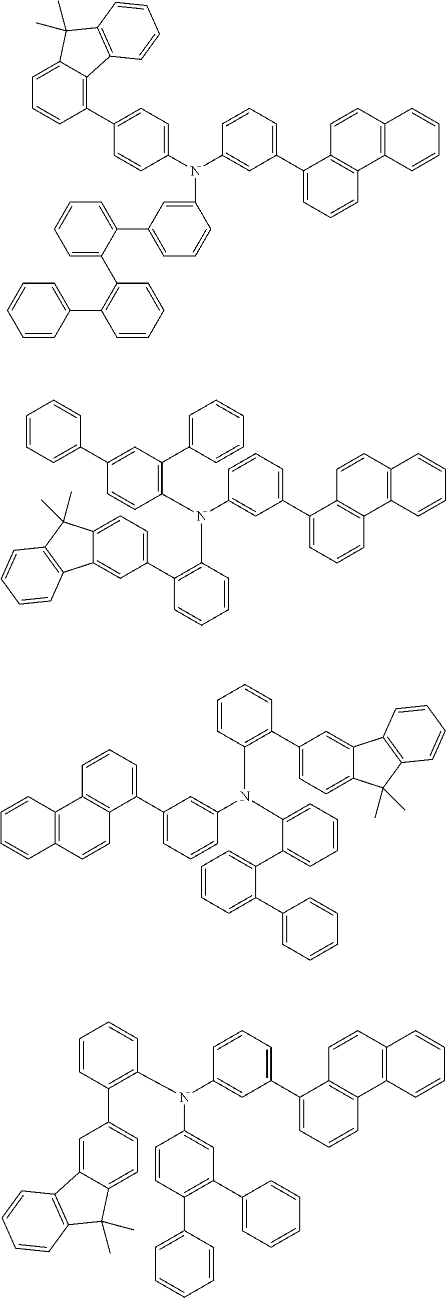 [SMILES: CC1(C)c2ccccc2-c2c(-c3ccc(N(c4cccc(-c5ccccc5-c5ccccc5-c5ccccc5)c4)c4cccc(-c5cccc6c5ccc5ccccc56)c4)cc3)cccc21 ; CC1(C)c2ccccc2-c2cc(-c3ccccc3N(c3cccc(-c4cccc5c4ccc4ccccc45)c3)c3ccc(-c4ccccc4)cc3-c3ccccc3)ccc21 ; CC1(C)c2ccccc2-c2cc(-c3ccccc3N(c3cccc(-c4cccc5c4ccc4ccccc45)c3)c3ccccc3-c3ccccc3-c3ccccc3)ccc21 ; CC1(C)c2ccccc2-c2cc(-c3ccccc3N(c3cccc(-c4cccc5c4ccc4ccccc45)c3)c3ccc(-c4ccccc4)c(-c4ccccc4)c3)ccc21]

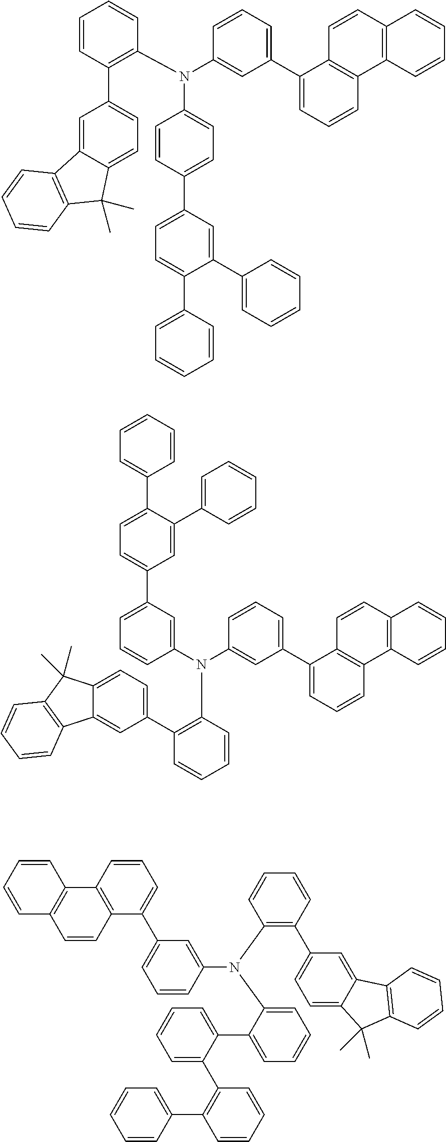 [SMILES: CC1(C)c2ccccc2-c2cc(-c3ccccc3N(c3ccc(-c4ccc(-c5ccccc5)c(-c5ccccc5)c4)cc3)c3cccc(-c4cccc5c4ccc4ccccc45)c3)ccc21 ; CC1(C)c2ccccc2-c2cc(-c3ccccc3N(c3cccc(-c4ccc(-c5ccccc5)c(-c5ccccc5)c4)c3)c3cccc(-c4cccc5c4ccc4ccccc45)c3)ccc21 ; CC1(C)c2ccccc2-c2cc(-c3ccccc3N(c3cccc(-c4cccc5c4ccc4ccccc45)c3)c3ccccc3-c3ccccc3-c3ccccc3-c3ccccc3)ccc21]

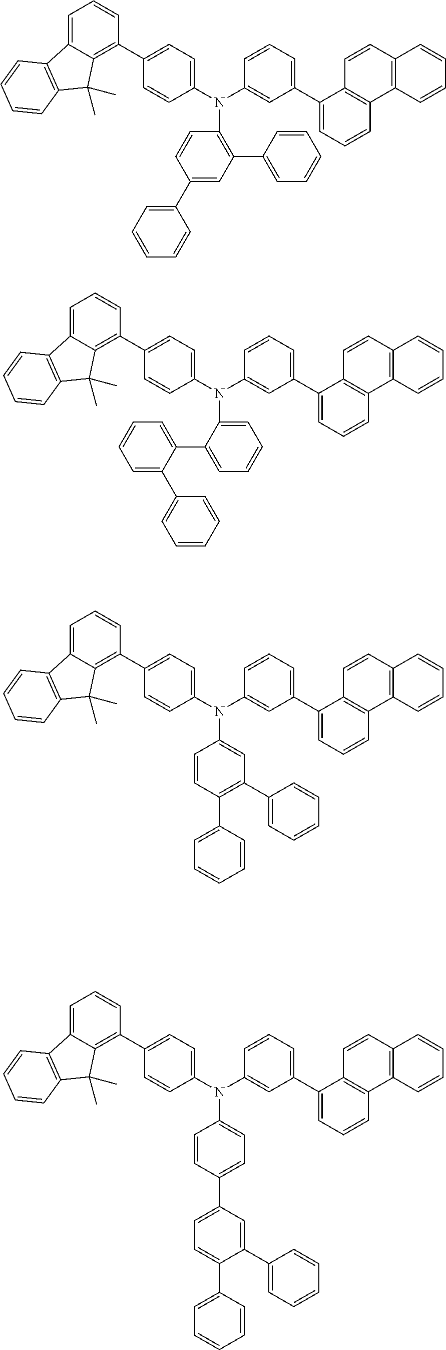 [SMILES: CC1(C)c2ccccc2-c2cccc(-c3ccc(N(c4cccc(-c5cccc6c5ccc5ccccc56)c4)c4ccc(-c5ccccc5)cc4-c4ccccc4)cc3)c21 ; CC1(C)c2ccccc2-c2cccc(-c3ccc(N(c4cccc(-c5cccc6c5ccc5ccccc56)c4)c4ccccc4-c4ccccc4-c4ccccc4)cc3)c21 ; CC1(C)c2ccccc2-c2cccc(-c3ccc(N(c4cccc(-c5cccc6c5ccc5ccccc56)c4)c4ccc(-c5ccccc5)c(-c5ccccc5)c4)cc3)c21 ; CC1(C)c2ccccc2-c2cccc(-c3ccc(N(c4ccc(-c5ccc(-c6ccccc6)c(-c6ccccc6)c5)cc4)c4cccc(-c5cccc6c5ccc5ccccc56)c4)cc3)c21]

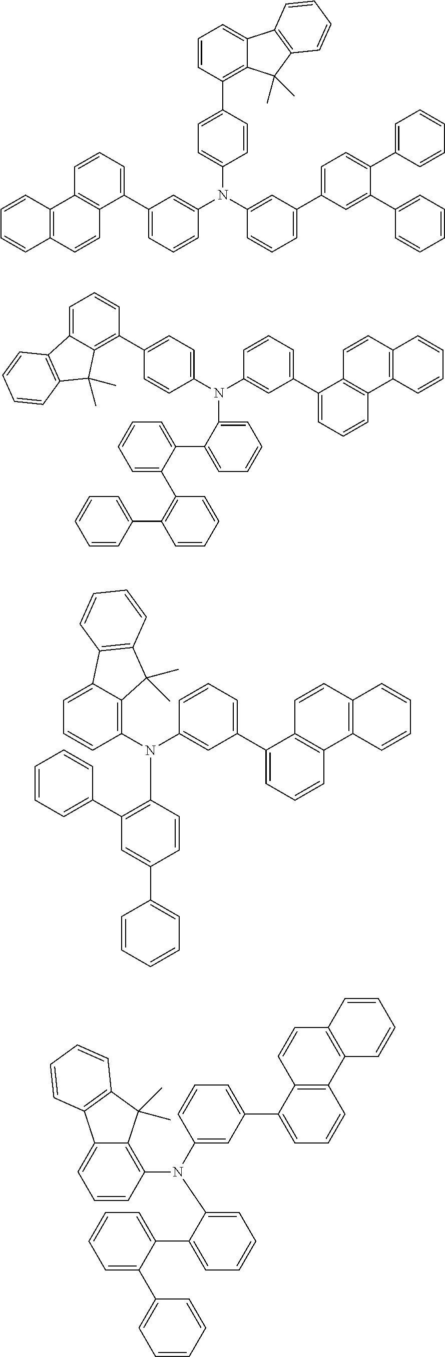 [SMILES: CC1(C)c2ccccc2-c2cccc(-c3ccc(N(c4cccc(-c5ccc(-c6ccccc6)c(-c6ccccc6)c5)c4)c4cccc(-c5cccc6c5ccc5ccccc56)c4)cc3)c21 ; CC1(C)c2ccccc2-c2cccc(-c3ccc(N(c4cccc(-c5cccc6c5ccc5ccccc56)c4)c4ccccc4-c4ccccc4-c4ccccc4-c4ccccc4)cc3)c21 ; CC1(C)c2ccccc2-c2cccc(N(c3cccc(-c4cccc5c4ccc4ccccc45)c3)c3ccc(-c4ccccc4)cc3-c3ccccc3)c21 ; CC1(C)c2ccccc2-c2cccc(N(c3cccc(-c4cccc5c4ccc4ccccc45)c3)c3ccccc3-c3ccccc3-c3ccccc3)c21]

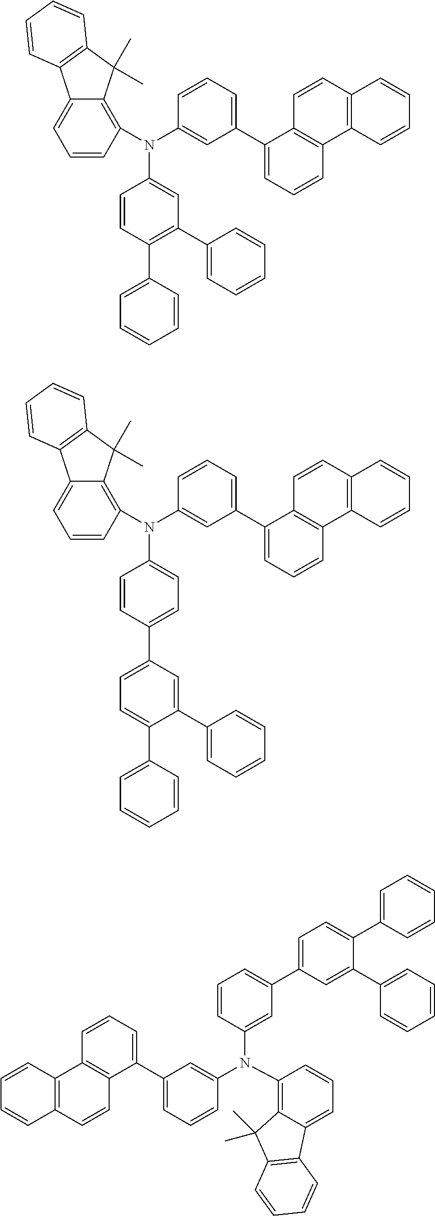 [SMILES: CC1(C)c2ccccc2-c2cccc(N(c3cccc(-c4cccc5c4ccc4ccccc45)c3)c3ccc(-c4ccccc4)c(-c4ccccc4)c3)c21 ; CC1(C)c2ccccc2-c2cccc(N(c3ccc(-c4ccc(-c5ccccc5)c(-c5ccccc5)c4)cc3)c3cccc(-c4cccc5c4ccc4ccccc45)c3)c21 ; CC1(C)c2ccccc2-c2cccc(N(c3cccc(-c4ccc(-c5ccccc5)c(-c5ccccc5)c4)c3)c3cccc(-c4cccc5c4ccc4ccccc45)c3)c21]

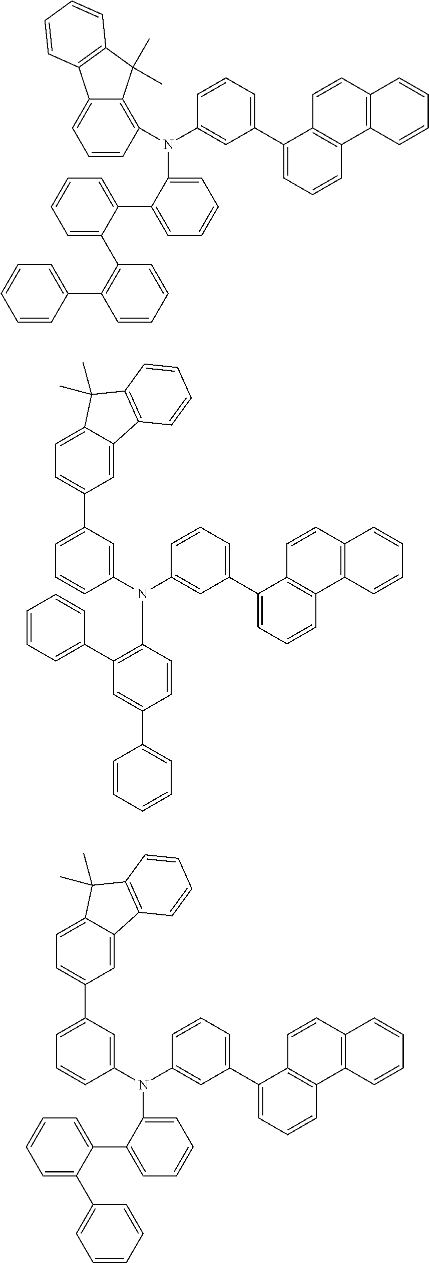

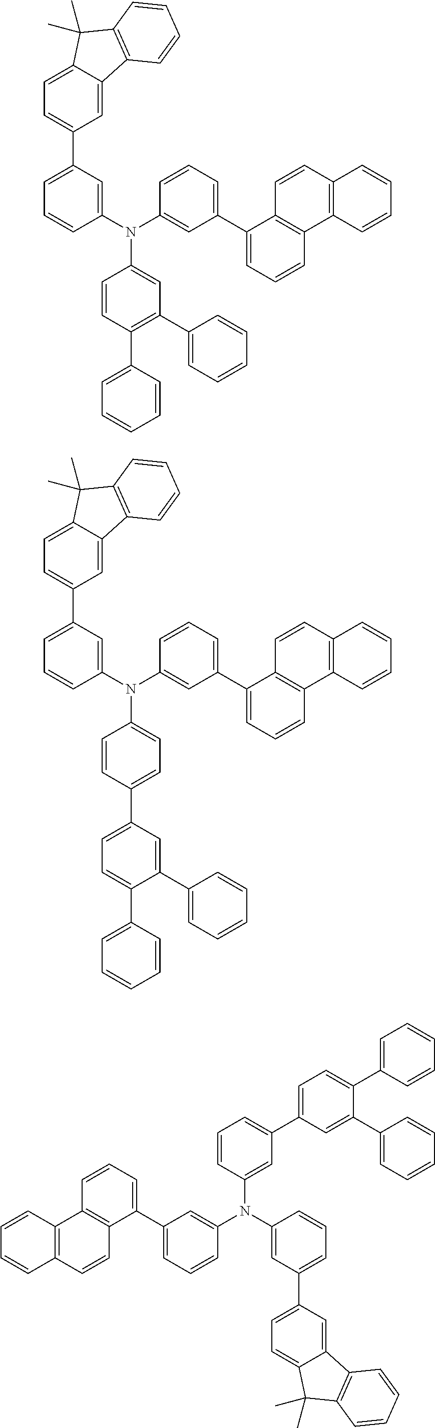

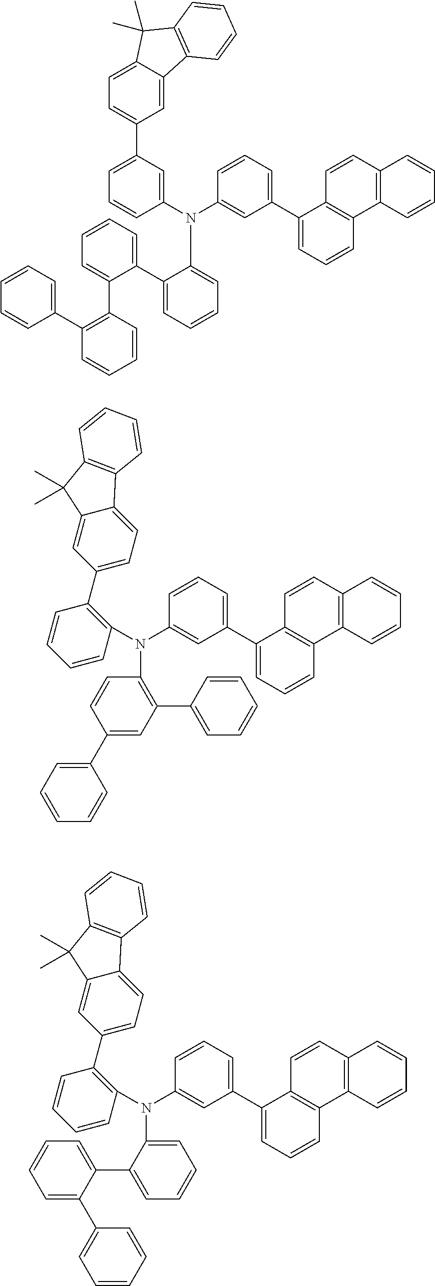

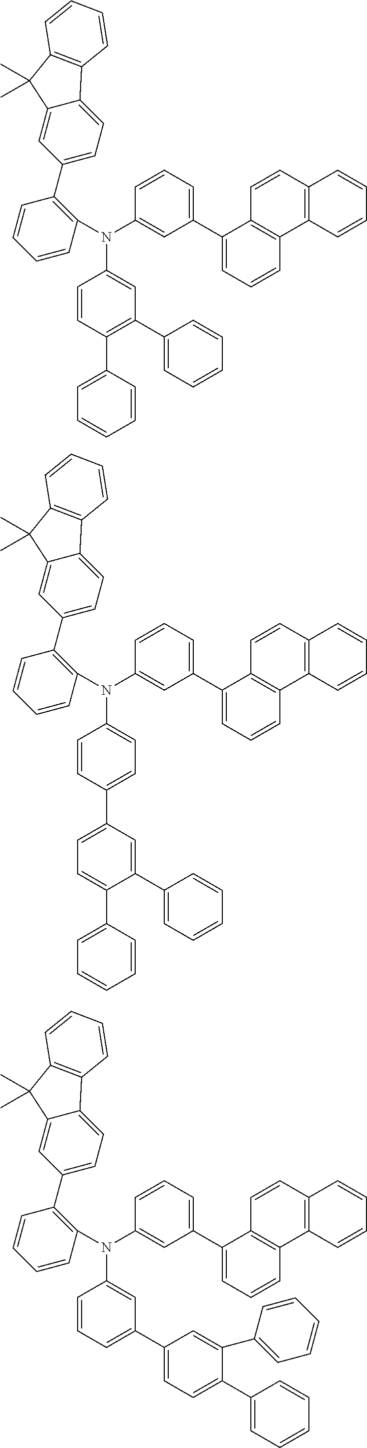

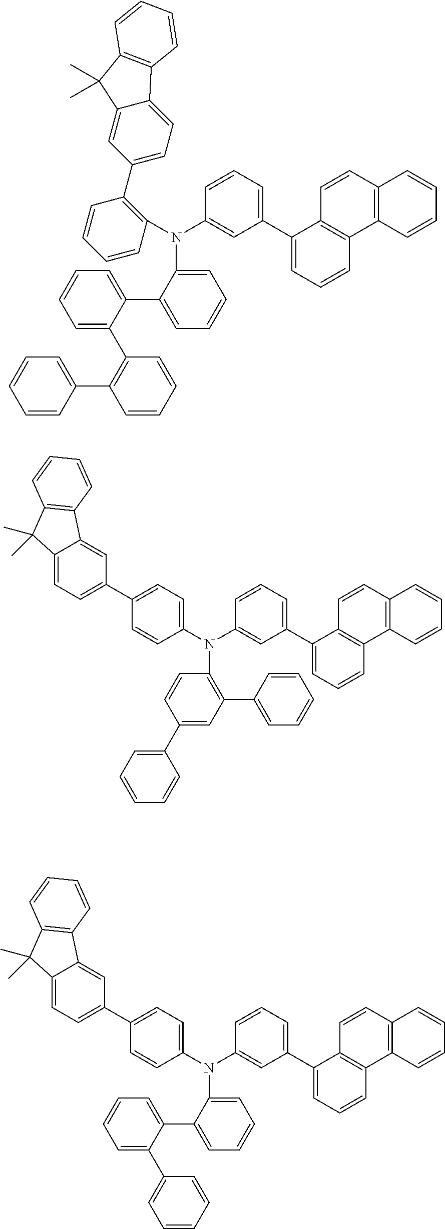 [SMILES: CC1(C)c2ccccc2-c2ccc(-c3ccccc3N(c3cccc(-c4cccc5c4ccc4ccccc45)c3)c3ccccc3-c3ccccc3-c3ccccc3-c3ccccc3)cc21 ; CC1(C)c2ccccc2-c2cc(-c3ccc(N(c4cccc(-c5cccc6c5ccc5ccccc56)c4)c4ccc(-c5ccccc5)cc4-c4ccccc4)cc3)ccc21 ; CC1(C)c2ccccc2-c2cc(-c3ccc(N(c4cccc(-c5cccc6c5ccc5ccccc56)c4)c4ccccc4-c4ccccc4-c4ccccc4)cc3)ccc21]

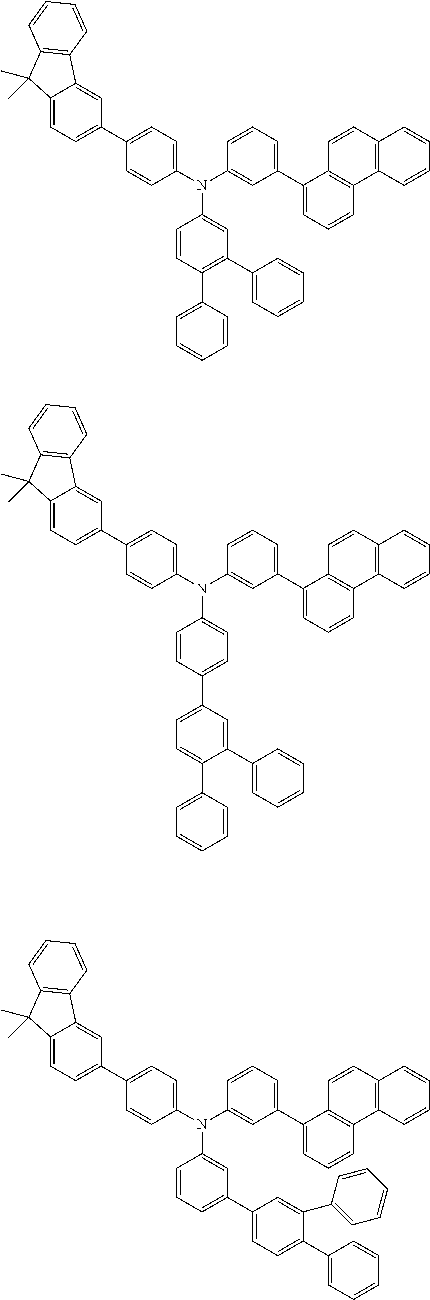 [SMILES: CC1(C)c2ccccc2-c2cc(-c3ccc(N(c4cccc(-c5cccc6c5ccc5ccccc56)c4)c4ccc(-c5ccccc5)c(-c5ccccc5)c4)cc3)ccc21 ; CC1(C)c2ccccc2-c2cc(-c3ccc(N(c4ccc(-c5ccc(-c6ccccc6)c(-c6ccccc6)c5)cc4)c4cccc(-c5cccc6c5ccc5ccccc56)c4)cc3)ccc21 ; CC1(C)c2ccccc2-c2cc(-c3ccc(N(c4cccc(-c5ccc(-c6ccccc6)c(-c6ccccc6)c5)c4)c4cccc(-c5cccc6c5ccc5ccccc56)c4)cc3)ccc21]

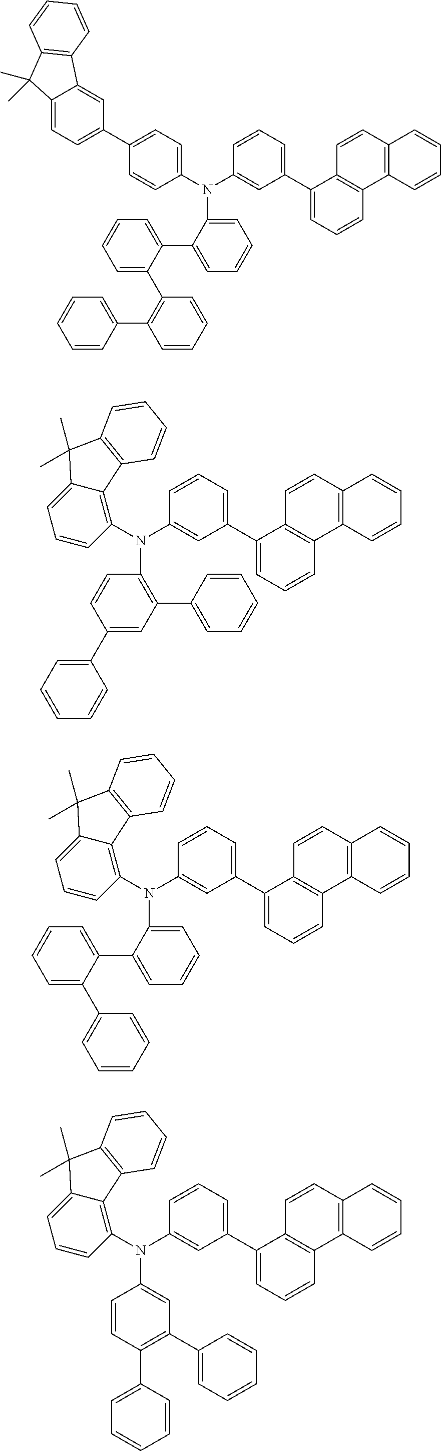

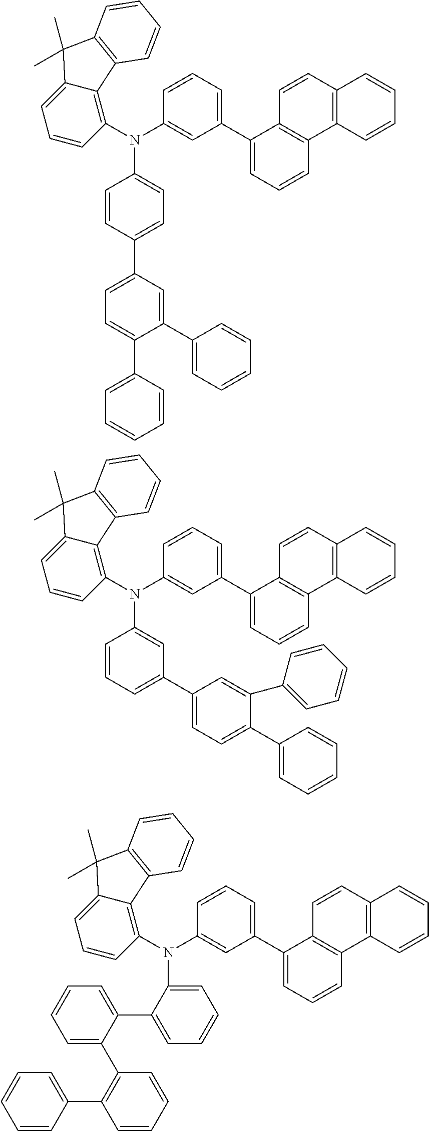

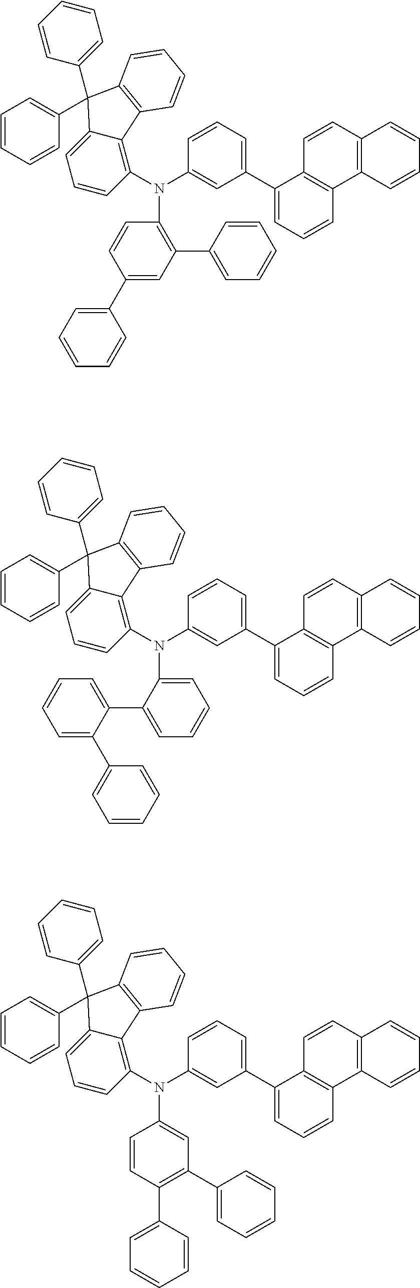 [SMILES: c1ccc(-c2ccc(N(c3cccc(-c4cccc5c4ccc4ccccc45)c3)c3cccc4c3-c3ccccc3C4(c3ccccc3)c3ccccc3)c(-c3ccccc3)c2)cc1 ; c1ccc(-c2ccccc2-c2ccccc2N(c2cccc(-c3cccc4c3ccc3ccccc34)c2)c2cccc3c2-c2ccccc2C3(c2ccccc2)c2ccccc2)cc1 ; c1ccc(-c2ccc(N(c3cccc(-c4cccc5c4ccc4ccccc45)c3)c3cccc4c3-c3ccccc3C4(c3ccccc3)c3ccccc3)cc2-c2ccccc2)cc1]

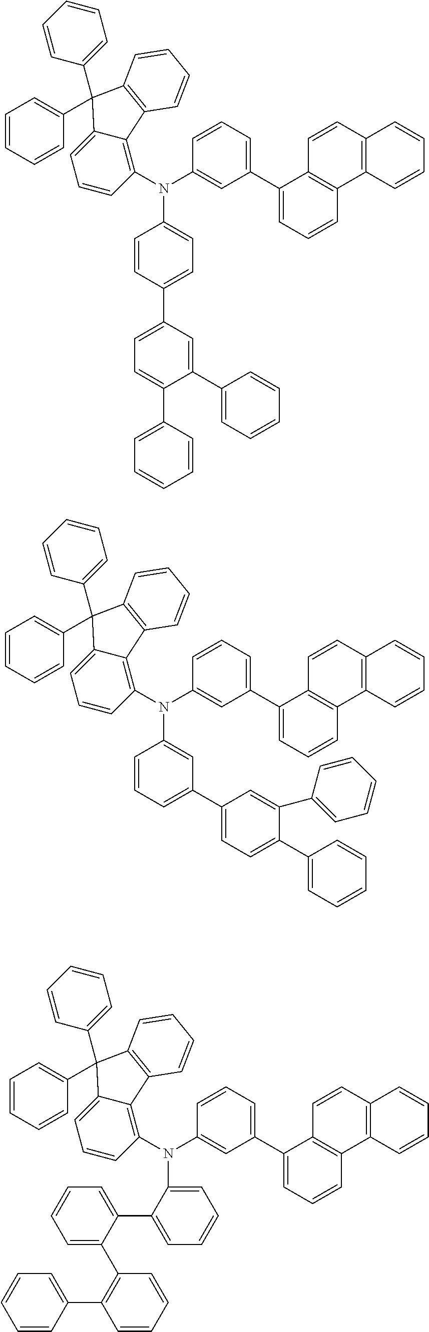 [SMILES: c1ccc(-c2ccc(-c3ccc(N(c4cccc(-c5cccc6c5ccc5ccccc56)c4)c4cccc5c4-c4ccccc4C5(c4ccccc4)c4ccccc4)cc3)cc2-c2ccccc2)cc1 ; c1ccc(-c2ccc(-c3cccc(N(c4cccc(-c5cccc6c5ccc5ccccc56)c4)c4cccc5c4-c4ccccc4C5(c4ccccc4)c4ccccc4)c3)cc2-c2ccccc2)cc1 ; c1ccc(-c2ccccc2-c2ccccc2-c2ccccc2N(c2cccc(-c3cccc4c3ccc3ccccc34)c2)c2cccc3c2-c2ccccc2C3(c2ccccc2)c2ccccc2)cc1]

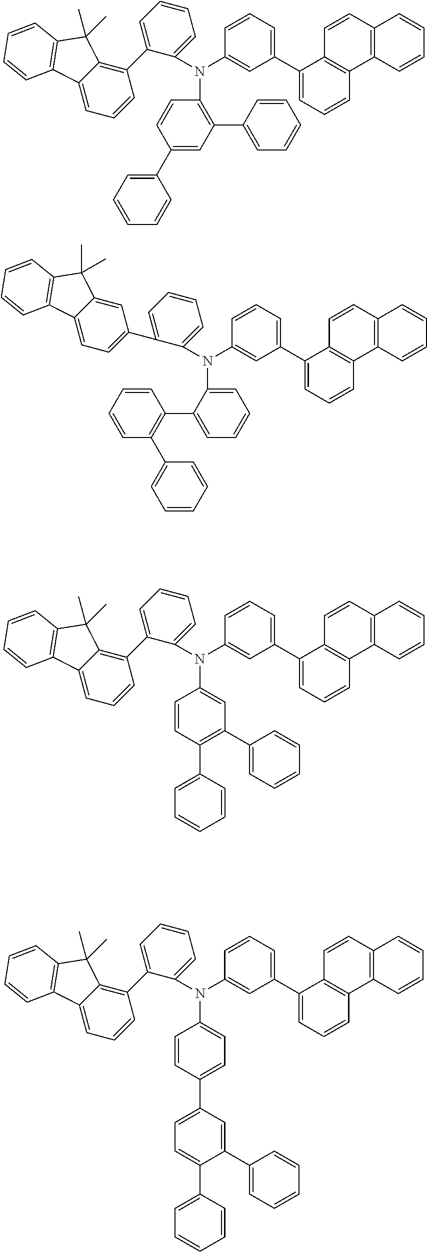 [SMILES: CC1(C)c2ccccc2-c2cccc(-c3ccccc3N(c3cccc(-c4cccc5c4ccc4ccccc45)c3)c3ccc(-c4ccccc4)cc3-c3ccccc3)c21 ; CC1(C)c2ccccc2-c2ccc(-c3ccccc3N(c3cccc(-c4cccc5c4ccc4ccccc45)c3)c3ccccc3-c3ccccc3-c3ccccc3)cc21 ; CC1(C)c2ccccc2-c2cccc(-c3ccccc3N(c3cccc(-c4cccc5c4ccc4ccccc45)c3)c3ccc(-c4ccccc4)c(-c4ccccc4)c3)c21 ; CC1(C)c2ccccc2-c2cccc(-c3ccccc3N(c3ccc(-c4ccc(-c5ccccc5)c(-c5ccccc5)c4)cc3)c3cccc(-c4cccc5c4ccc4ccccc45)c3)c21]

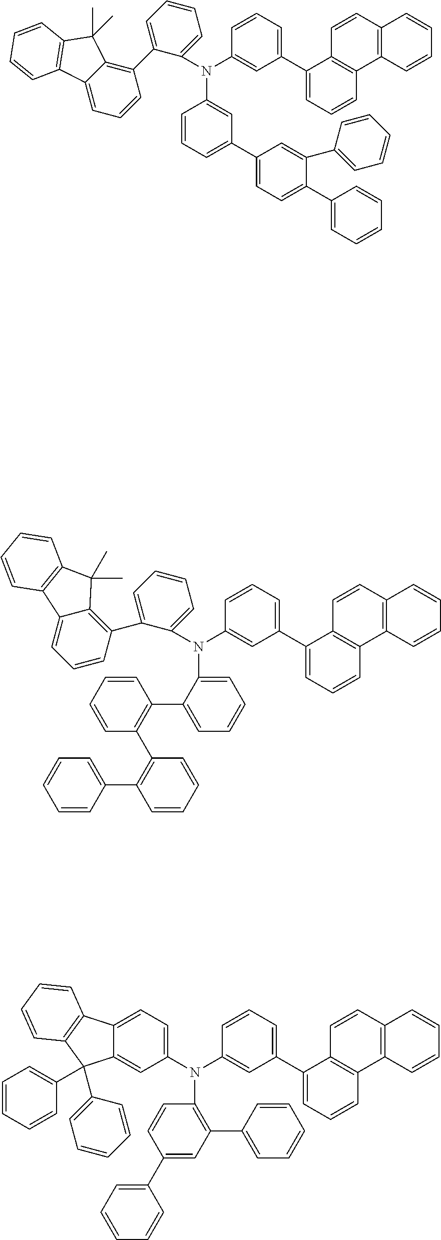 [SMILES: CC1(C)c2ccccc2-c2cccc(-c3ccccc3N(c3cccc(-c4ccc(-c5ccccc5)c(-c5ccccc5)c4)c3)c3cccc(-c4cccc5c4ccc4ccccc45)c3)c21 ; CC1(C)c2ccccc2-c2cccc(-c3ccccc3N(c3cccc(-c4cccc5c4ccc4ccccc45)c3)c3ccccc3-c3ccccc3-c3ccccc3-c3ccccc3)c21 ; c1ccc(-c2ccc(N(c3cccc(-c4cccc5c4ccc4ccccc45)c3)c3ccc4c(c3)C(c3ccccc3)(c3ccccc3)c3ccccc3-4)c(-c3ccccc3)c2)cc1]

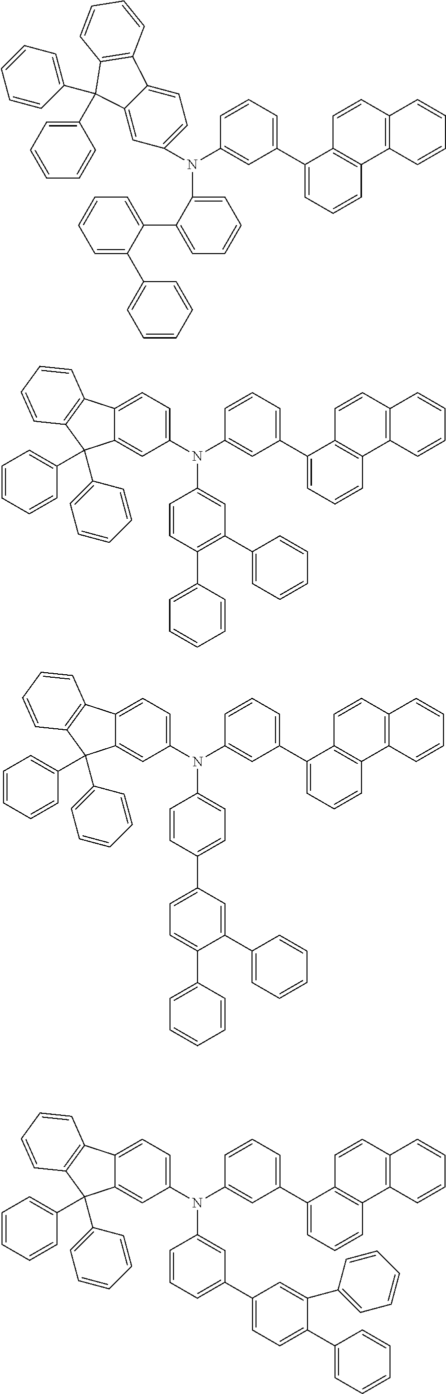 [SMILES: c1ccc(-c2ccccc2-c2ccccc2N(c2cccc(-c3cccc4c3ccc3ccccc34)c2)c2ccc3c(c2)C(c2ccccc2)(c2ccccc2)c2ccccc2-3)cc1 ; c1ccc(-c2ccc(N(c3cccc(-c4cccc5c4ccc4ccccc45)c3)c3ccc4c(c3)C(c3ccccc3)(c3ccccc3)c3ccccc3-4)cc2-c2ccccc2)cc1 ; c1ccc(-c2ccc(-c3ccc(N(c4cccc(-c5cccc6c5ccc5ccccc56)c4)c4ccc5c(c4)C(c4ccccc4)(c4ccccc4)c4ccccc4-5)cc3)cc2-c2ccccc2)cc1 ; c1ccc(-c2ccc(-c3cccc(N(c4cccc(-c5cccc6c5ccc5ccccc56)c4)c4ccc5c(c4)C(c4ccccc4)(c4ccccc4)c4ccccc4-5)c3)cc2-c2ccccc2)cc1]

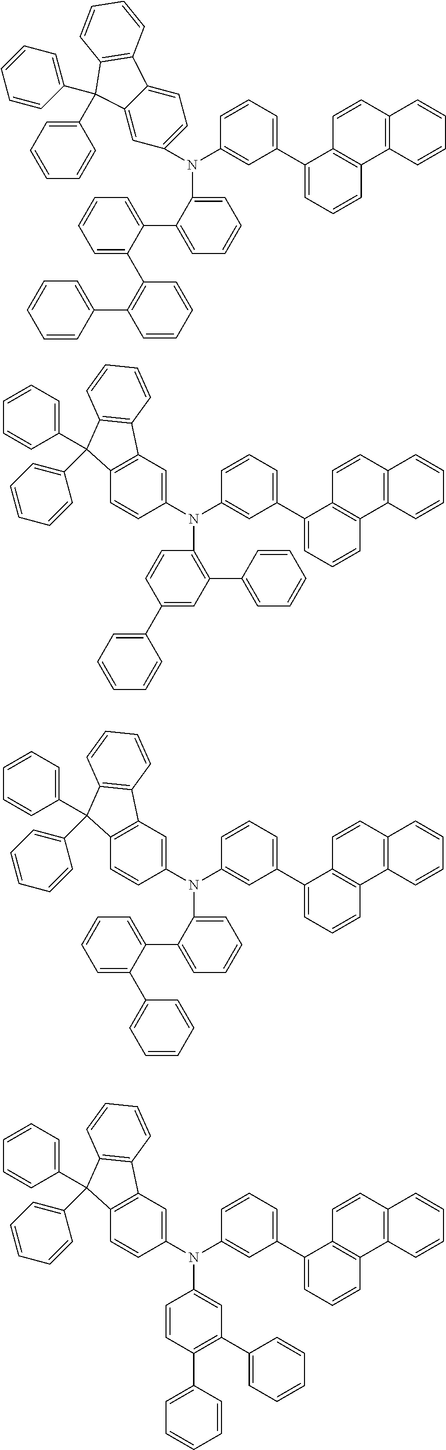

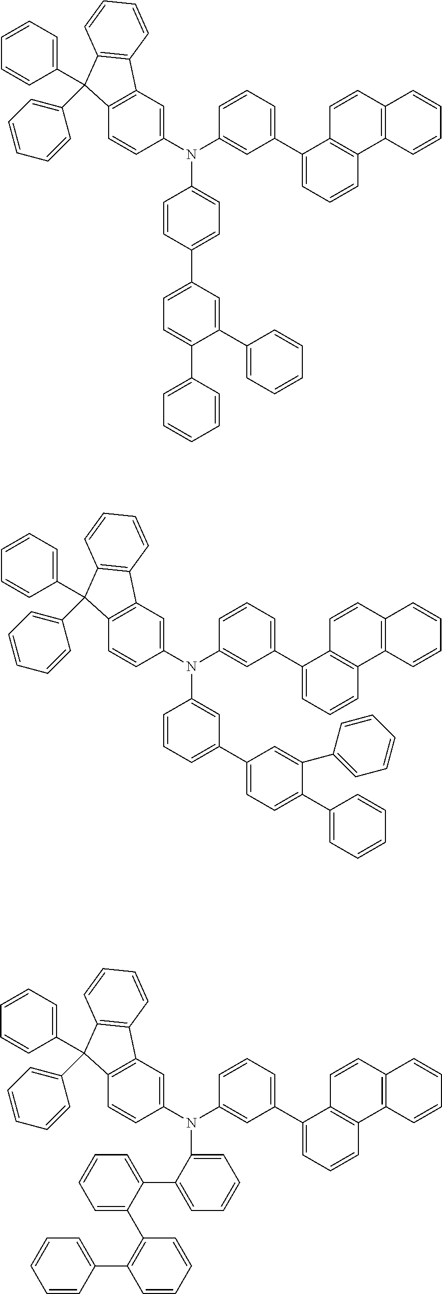 [SMILES: c1ccc(-c2ccc(-c3ccc(N(c4cccc(-c5cccc6c5ccc5ccccc56)c4)c4ccc5c(c4)-c4ccccc4C5(c4ccccc4)c4ccccc4)cc3)cc2-c2ccccc2)cc1 ; c1ccc(-c2ccc(-c3cccc(N(c4cccc(-c5cccc6c5ccc5ccccc56)c4)c4ccc5c(c4)-c4ccccc4C5(c4ccccc4)c4ccccc4)c3)cc2-c2ccccc2)cc1 ; c1ccc(-c2ccccc2-c2ccccc2-c2ccccc2N(c2cccc(-c3cccc4c3ccc3ccccc34)c2)c2ccc3c(c2)-c2ccccc2C3(c2ccccc2)c2ccccc2)cc1]

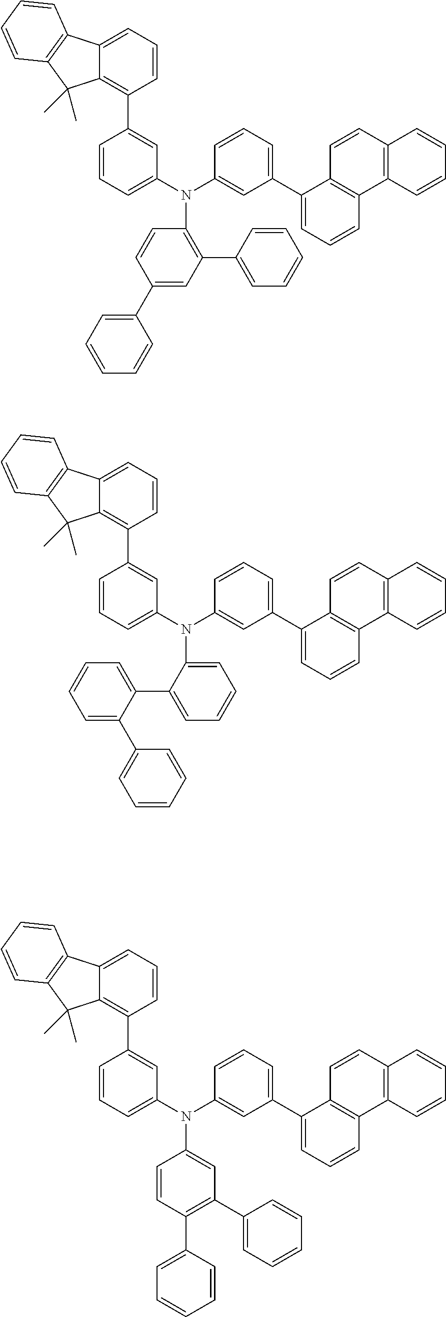 [SMILES: CC1(C)c2ccccc2-c2cccc(-c3cccc(N(c4cccc(-c5cccc6c5ccc5ccccc56)c4)c4ccc(-c5ccccc5)cc4-c4ccccc4)c3)c21 ; CC1(C)c2ccccc2-c2cccc(-c3cccc(N(c4cccc(-c5cccc6c5ccc5ccccc56)c4)c4ccccc4-c4ccccc4-c4ccccc4)c3)c21 ; CC1(C)c2ccccc2-c2cccc(-c3cccc(N(c4cccc(-c5cccc6c5ccc5ccccc56)c4)c4ccc(-c5ccccc5)c(-c5ccccc5)c4)c3)c21]

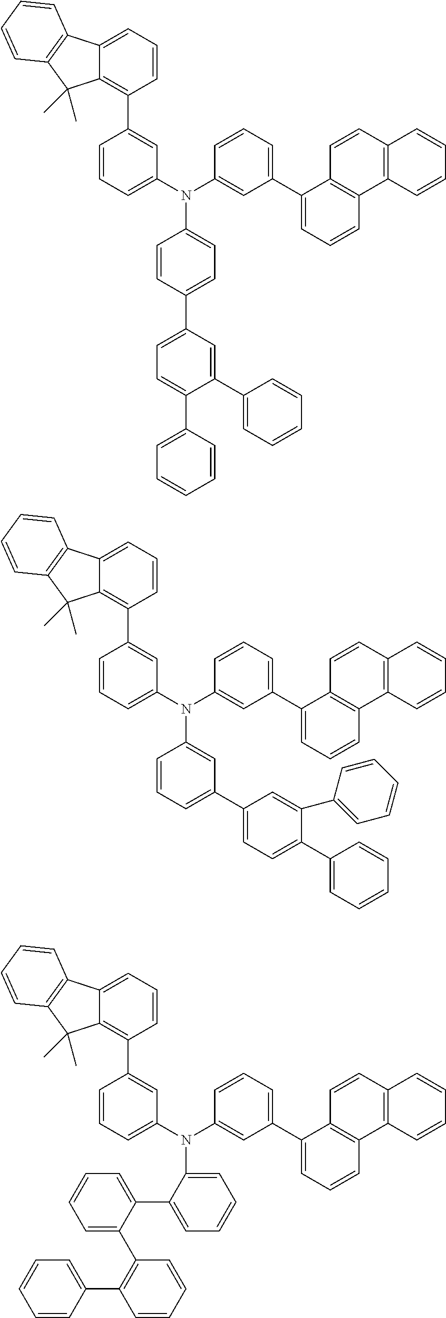 [SMILES: CC1(C)c2ccccc2-c2cccc(-c3cccc(N(c4ccc(-c5ccc(-c6ccccc6)c(-c6ccccc6)c5)cc4)c4cccc(-c5cccc6c5ccc5ccccc56)c4)c3)c21 ; CC1(C)c2ccccc2-c2cccc(-c3cccc(N(c4cccc(-c5ccc(-c6ccccc6)c(-c6ccccc6)c5)c4)c4cccc(-c5cccc6c5ccc5ccccc56)c4)c3)c21 ; CC1(C)c2ccccc2-c2cccc(-c3cccc(N(c4cccc(-c5cccc6c5ccc5ccccc56)c4)c4ccccc4-c4ccccc4-c4ccccc4-c4ccccc4)c3)c21]

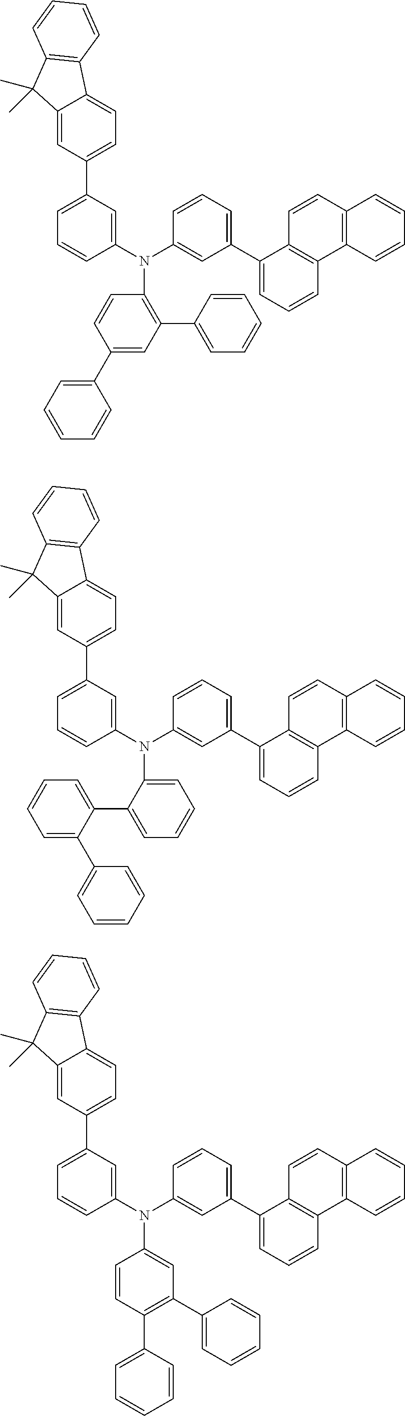

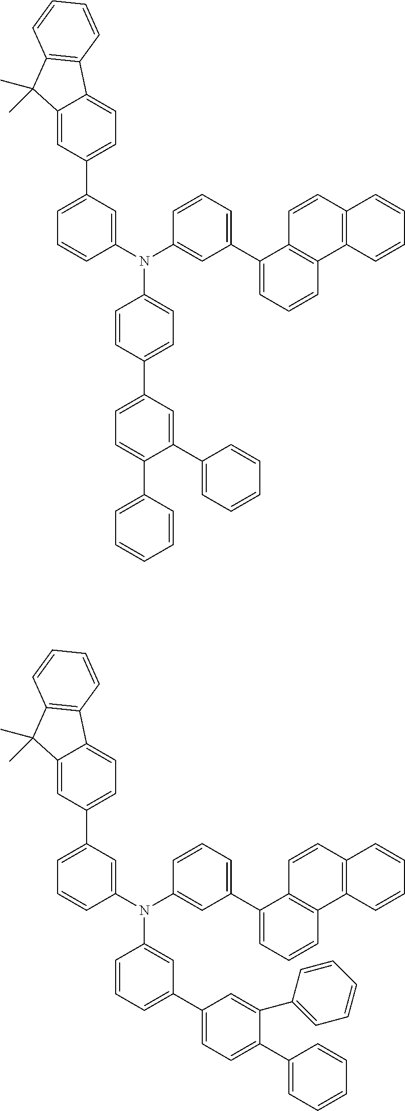 [SMILES: CC1(C)c2ccccc2-c2ccc(-c3cccc(N(c4ccc(-c5ccc(-c6ccccc6)c(-c6ccccc6)c5)cc4)c4cccc(-c5cccc6c5ccc5ccccc56)c4)c3)cc21 ; CC1(C)c2ccccc2-c2ccc(-c3cccc(N(c4cccc(-c5ccc(-c6ccccc6)c(-c6ccccc6)c5)c4)c4cccc(-c5cccc6c5ccc5ccccc56)c4)c3)cc21]

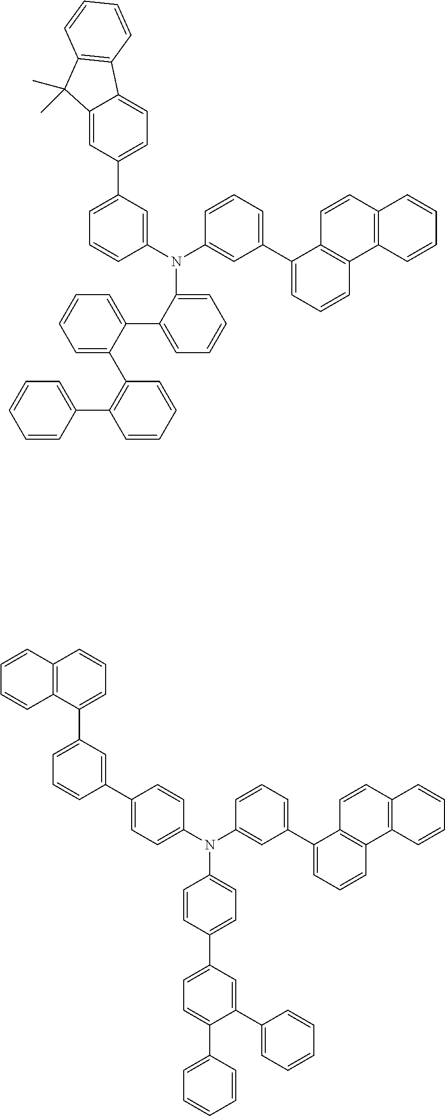 [SMILES: CC1(C)c2ccccc2-c2ccc(-c3cccc(N(c4cccc(-c5cccc6c5ccc5ccccc56)c4)c4ccccc4-c4ccccc4-c4ccccc4-c4ccccc4)c3)cc21 ; c1ccc(-c2ccc(-c3ccc(N(c4ccc(-c5cccc(-c6cccc7ccccc67)c5)cc4)c4cccc(-c5cccc6c5ccc5ccccc56)c4)cc3)cc2-c2ccccc2)cc1]

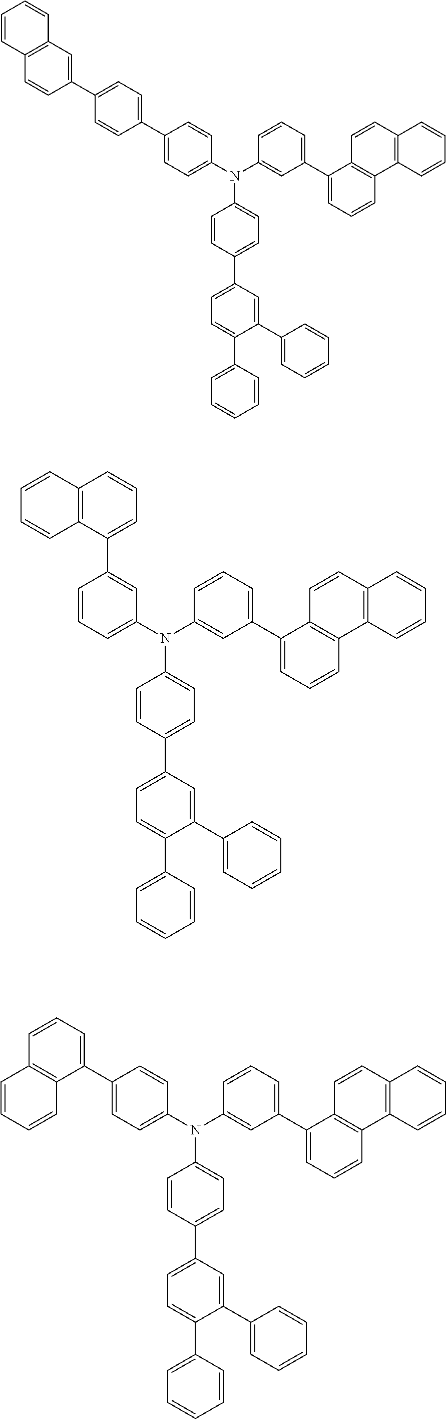 [SMILES: c1ccc(-c2ccc(-c3ccc(N(c4ccc(-c5ccc(-c6ccc7ccccc7c6)cc5)cc4)c4cccc(-c5cccc6c5ccc5ccccc56)c4)cc3)cc2-c2ccccc2)cc1 ; c1ccc(-c2ccc(-c3ccc(N(c4cccc(-c5cccc6ccccc56)c4)c4cccc(-c5cccc6c5ccc5ccccc56)c4)cc3)cc2-c2ccccc2)cc1 ; c1ccc(-c2ccc(-c3ccc(N(c4ccc(-c5cccc6ccccc56)cc4)c4cccc(-c5cccc6c5ccc5ccccc56)c4)cc3)cc2-c2ccccc2)cc1]

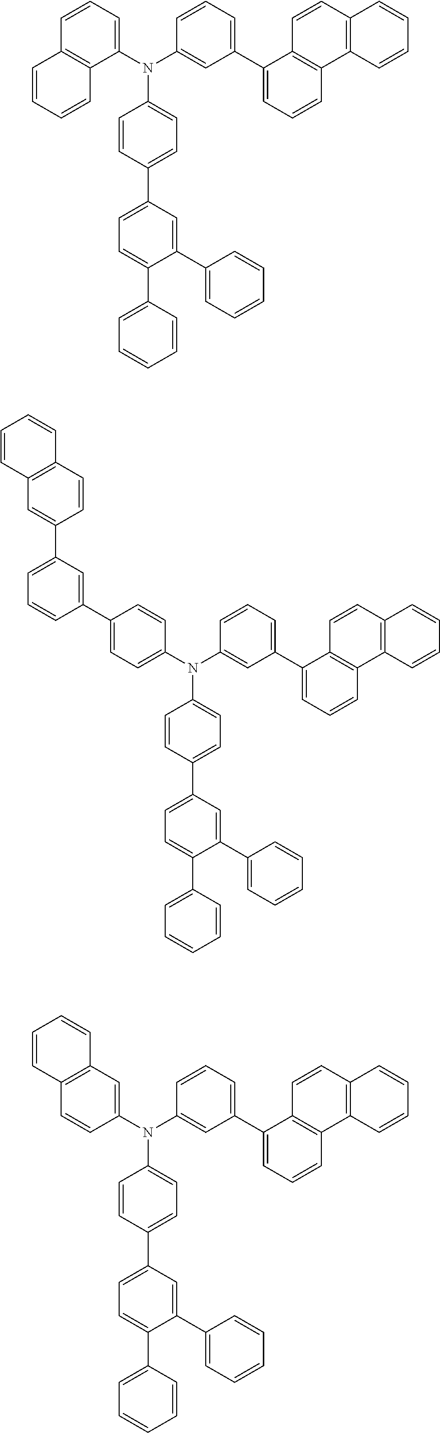 [SMILES: c1ccc(-c2ccc(-c3ccc(N(c4cccc(-c5cccc6c5ccc5ccccc56)c4)c4cccc5ccccc45)cc3)cc2-c2ccccc2)cc1 ; c1ccc(-c2ccc(-c3ccc(N(c4ccc(-c5cccc(-c6ccc7ccccc7c6)c5)cc4)c4cccc(-c5cccc6c5ccc5ccccc56)c4)cc3)cc2-c2ccccc2)cc1 ; c1ccc(-c2ccc(-c3ccc(N(c4cccc(-c5cccc6c5ccc5ccccc56)c4)c4ccc5ccccc5c4)cc3)cc2-c2ccccc2)cc1]

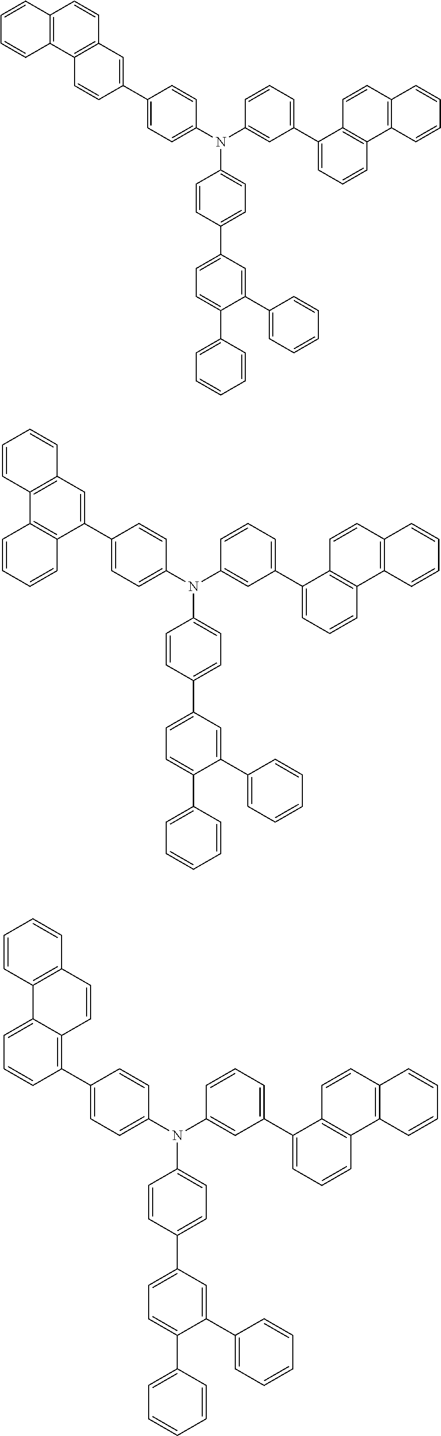 [SMILES: c1ccc(-c2ccc(-c3ccc(N(c4ccc(-c5ccc6c(ccc7ccccc76)c5)cc4)c4cccc(-c5cccc6c5ccc5ccccc56)c4)cc3)cc2-c2ccccc2)cc1 ; c1ccc(-c2ccc(-c3ccc(N(c4ccc(-c5cc6ccccc6c6ccccc56)cc4)c4cccc(-c5cccc6c5ccc5ccccc56)c4)cc3)cc2-c2ccccc2)cc1 ; c1ccc(-c2ccc(-c3ccc(N(c4ccc(-c5cccc6c5ccc5ccccc56)cc4)c4cccc(-c5cccc6c5ccc5ccccc56)c4)cc3)cc2-c2ccccc2)cc1]

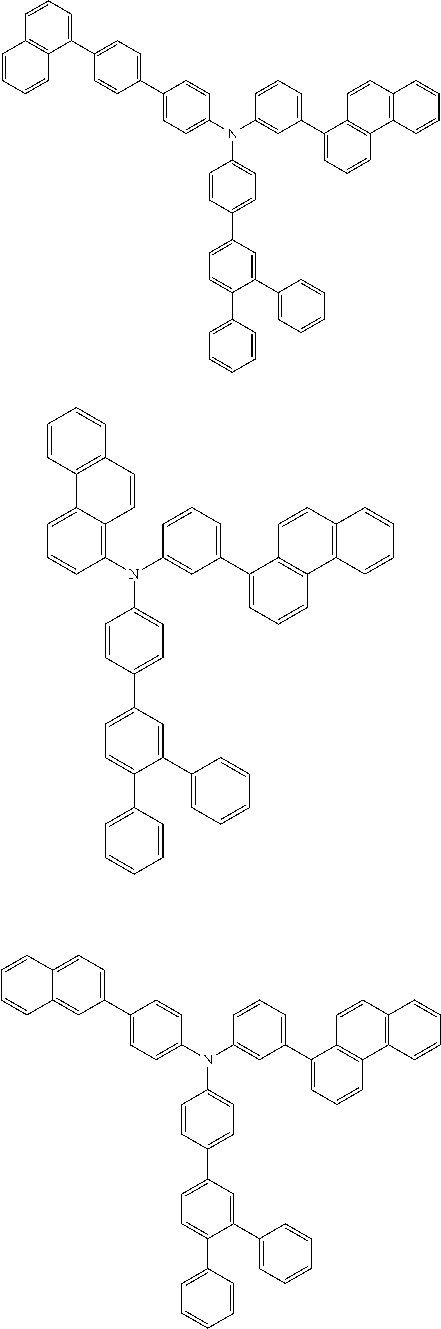 [SMILES: c1ccc(-c2ccc(-c3ccc(N(c4ccc(-c5ccc(-c6cccc7ccccc67)cc5)cc4)c4cccc(-c5cccc6c5ccc5ccccc56)c4)cc3)cc2-c2ccccc2)cc1 ; c1ccc(-c2ccc(-c3ccc(N(c4cccc(-c5cccc6c5ccc5ccccc56)c4)c4cccc5c4ccc4ccccc45)cc3)cc2-c2ccccc2)cc1 ; c1ccc(-c2ccc(-c3ccc(N(c4ccc(-c5ccc6ccccc6c5)cc4)c4cccc(-c5cccc6c5ccc5ccccc56)c4)cc3)cc2-c2ccccc2)cc1]

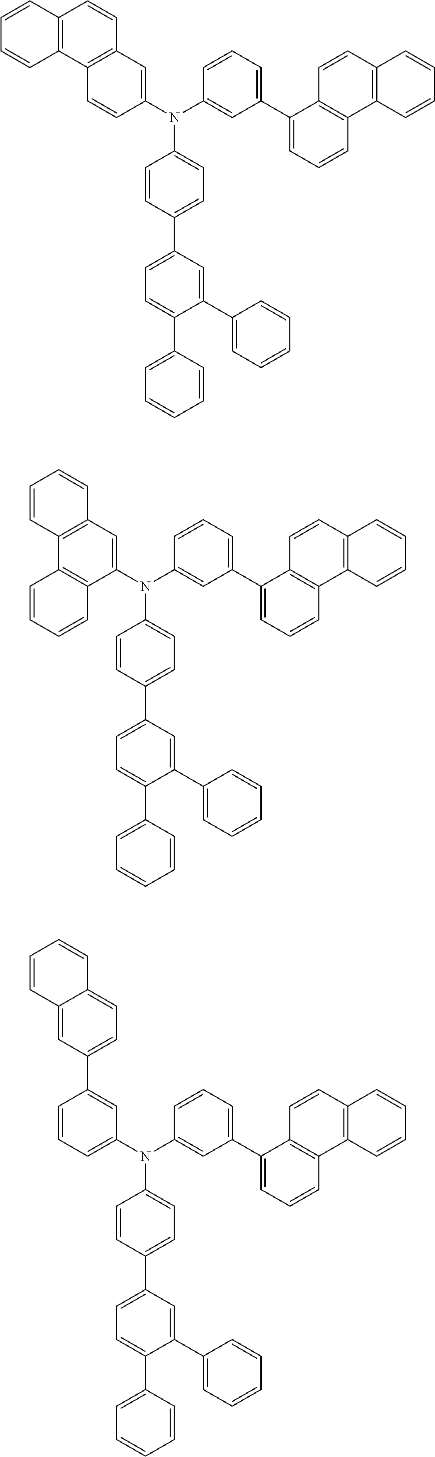 [SMILES: c1ccc(-c2ccc(-c3ccc(N(c4cccc(-c5cccc6c5ccc5ccccc56)c4)c4ccc5c(ccc6ccccc65)c4)cc3)cc2-c2ccccc2)cc1 ; c1ccc(-c2ccc(-c3ccc(N(c4cccc(-c5cccc6c5ccc5ccccc56)c4)c4cc5ccccc5c5ccccc45)cc3)cc2-c2ccccc2)cc1 ; c1ccc(-c2ccc(-c3ccc(N(c4cccc(-c5ccc6ccccc6c5)c4)c4cccc(-c5cccc6c5ccc5ccccc56)c4)cc3)cc2-c2ccccc2)cc1]

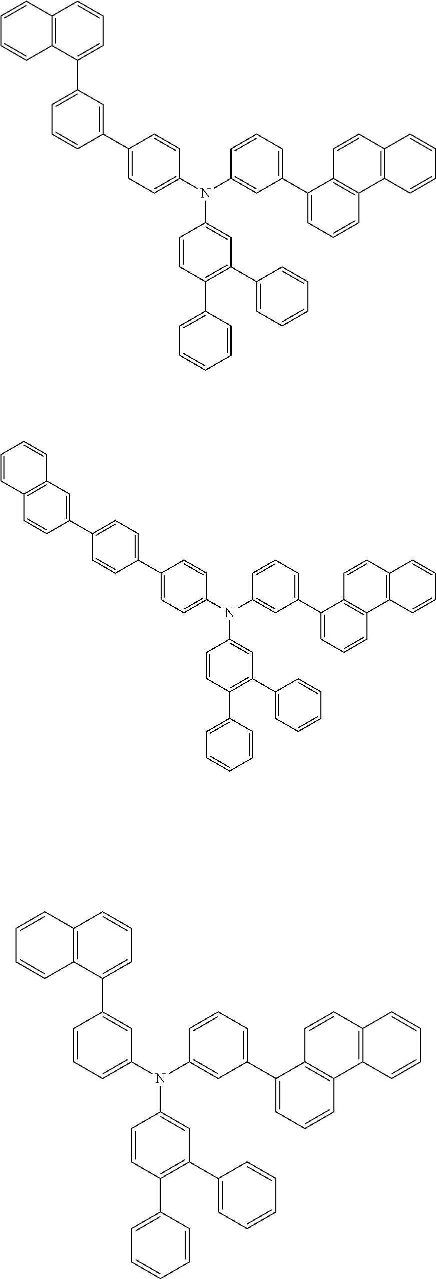 [SMILES: c1ccc(-c2ccc(N(c3ccc(-c4cccc(-c5cccc6ccccc56)c4)cc3)c3cccc(-c4cccc5c4ccc4ccccc45)c3)cc2-c2ccccc2)cc1 ; c1ccc(-c2ccc(N(c3ccc(-c4ccc(-c5ccc6ccccc6c5)cc4)cc3)c3cccc(-c4cccc5c4ccc4ccccc45)c3)cc2-c2ccccc2)cc1 ; c1ccc(-c2ccc(N(c3cccc(-c4cccc5ccccc45)c3)c3cccc(-c4cccc5c4ccc4ccccc45)c3)cc2-c2ccccc2)cc1]

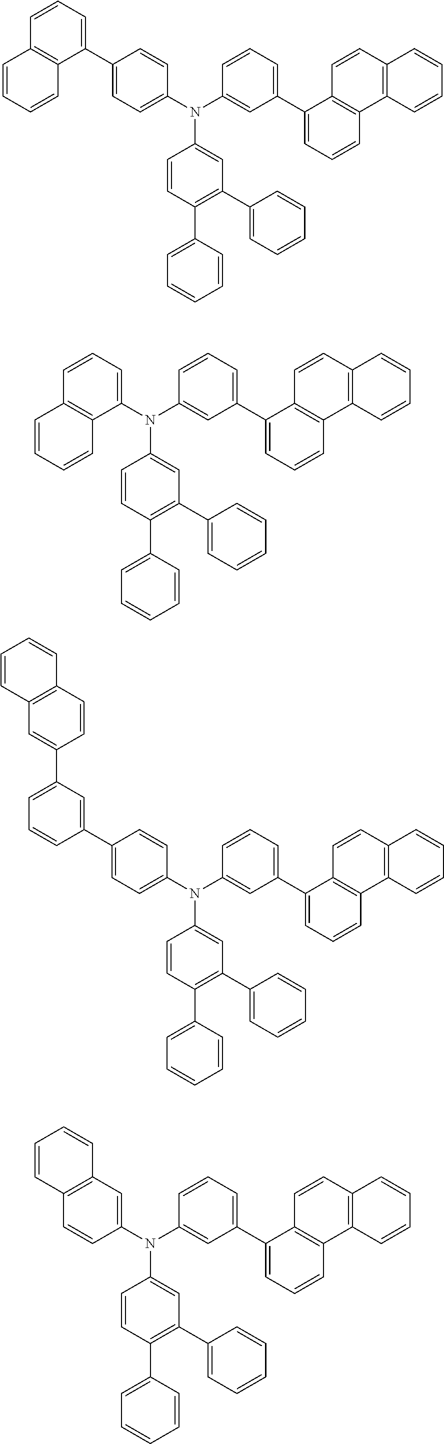 [SMILES: c1ccc(-c2ccc(N(c3ccc(-c4cccc5ccccc45)cc3)c3cccc(-c4cccc5c4ccc4ccccc45)c3)cc2-c2ccccc2)cc1 ; c1ccc(-c2ccc(N(c3cccc(-c4cccc5c4ccc4ccccc45)c3)c3cccc4ccccc34)cc2-c2ccccc2)cc1 ; c1ccc(-c2ccc(N(c3ccc(-c4cccc(-c5ccc6ccccc6c5)c4)cc3)c3cccc(-c4cccc5c4ccc4ccccc45)c3)cc2-c2ccccc2)cc1 ; c1ccc(-c2ccc(N(c3cccc(-c4cccc5c4ccc4ccccc45)c3)c3ccc4ccccc4c3)cc2-c2ccccc2)cc1]

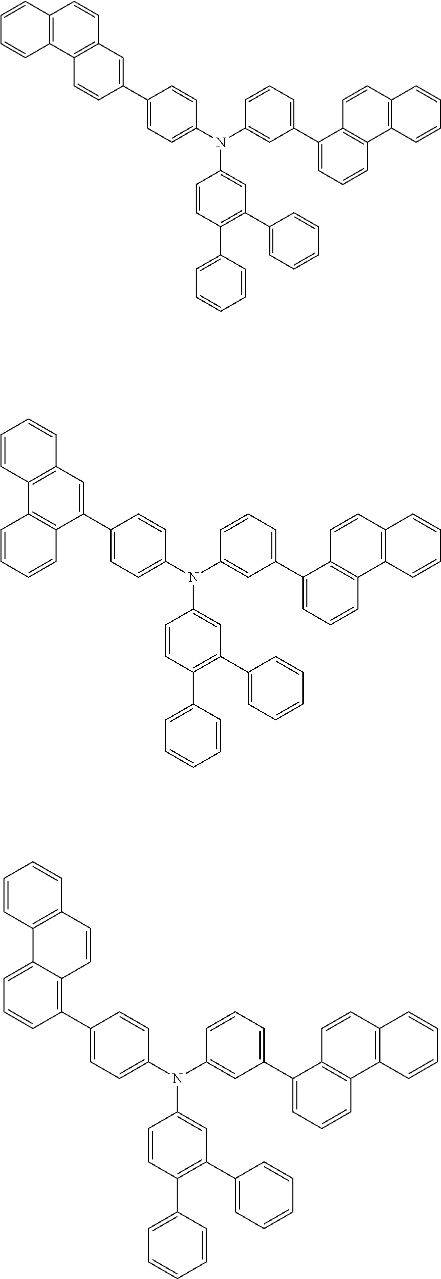 [SMILES: c1ccc(-c2ccc(N(c3ccc(-c4ccc5c(ccc6ccccc65)c4)cc3)c3cccc(-c4cccc5c4ccc4ccccc45)c3)cc2-c2ccccc2)cc1 ; c1ccc(-c2ccc(N(c3ccc(-c4cc5ccccc5c5ccccc45)cc3)c3cccc(-c4cccc5c4ccc4ccccc45)c3)cc2-c2ccccc2)cc1 ; c1ccc(-c2ccc(N(c3ccc(-c4cccc5c4ccc4ccccc45)cc3)c3cccc(-c4cccc5c4ccc4ccccc45)c3)cc2-c2ccccc2)cc1]

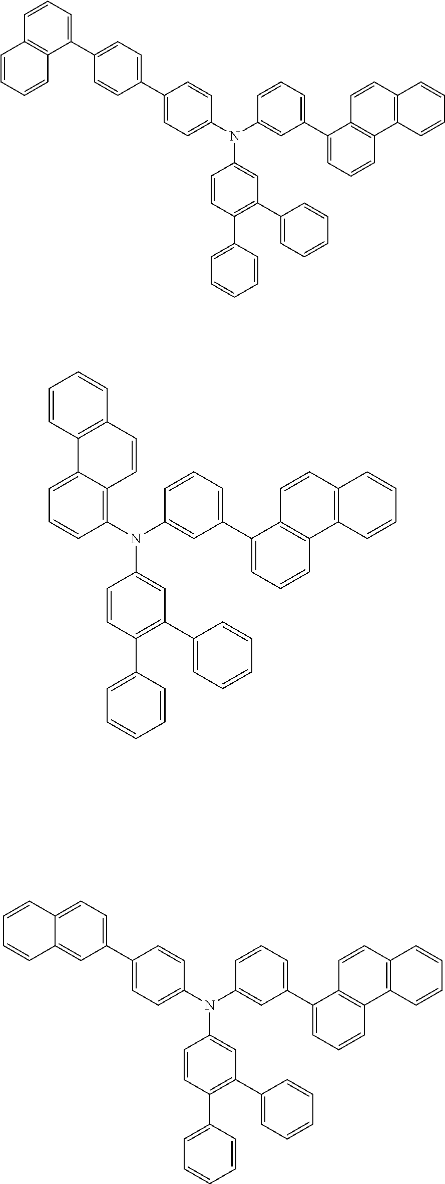 [SMILES: c1ccc(-c2ccc(N(c3ccc(-c4ccc(-c5cccc6ccccc56)cc4)cc3)c3cccc(-c4cccc5c4ccc4ccccc45)c3)cc2-c2ccccc2)cc1 ; c1ccc(-c2ccc(N(c3cccc(-c4cccc5c4ccc4ccccc45)c3)c3cccc4c3ccc3ccccc34)cc2-c2ccccc2)cc1 ; c1ccc(-c2ccc(N(c3ccc(-c4ccc5ccccc5c4)cc3)c3cccc(-c4cccc5c4ccc4ccccc45)c3)cc2-c2ccccc2)cc1]

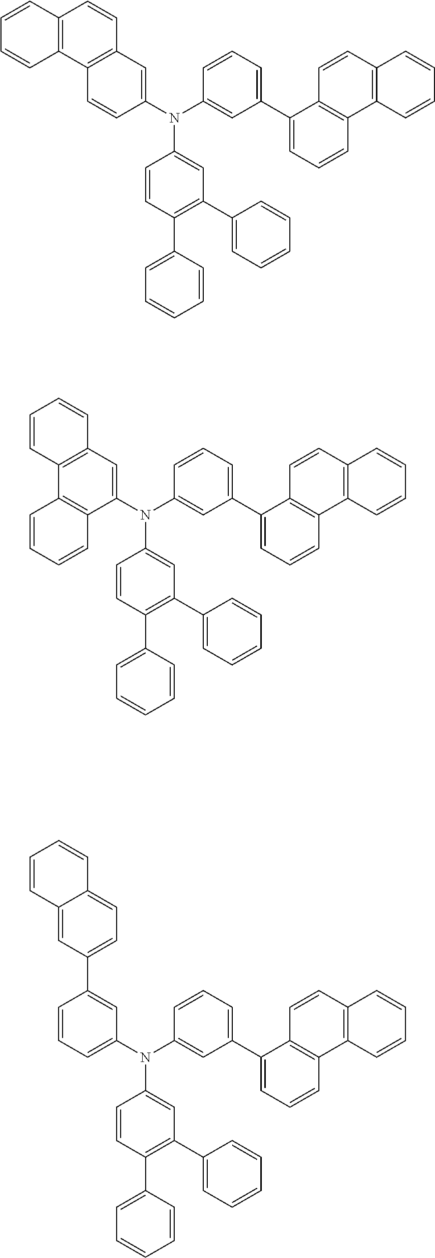 [SMILES: c1ccc(-c2ccc(N(c3cccc(-c4cccc5c4ccc4ccccc45)c3)c3ccc4c(ccc5ccccc54)c3)cc2-c2ccccc2)cc1 ; c1ccc(-c2ccc(N(c3cccc(-c4cccc5c4ccc4ccccc45)c3)c3cc4ccccc4c4ccccc34)cc2-c2ccccc2)cc1 ; c1ccc(-c2ccc(N(c3cccc(-c4ccc5ccccc5c4)c3)c3cccc(-c4cccc5c4ccc4ccccc45)c3)cc2-c2ccccc2)cc1]

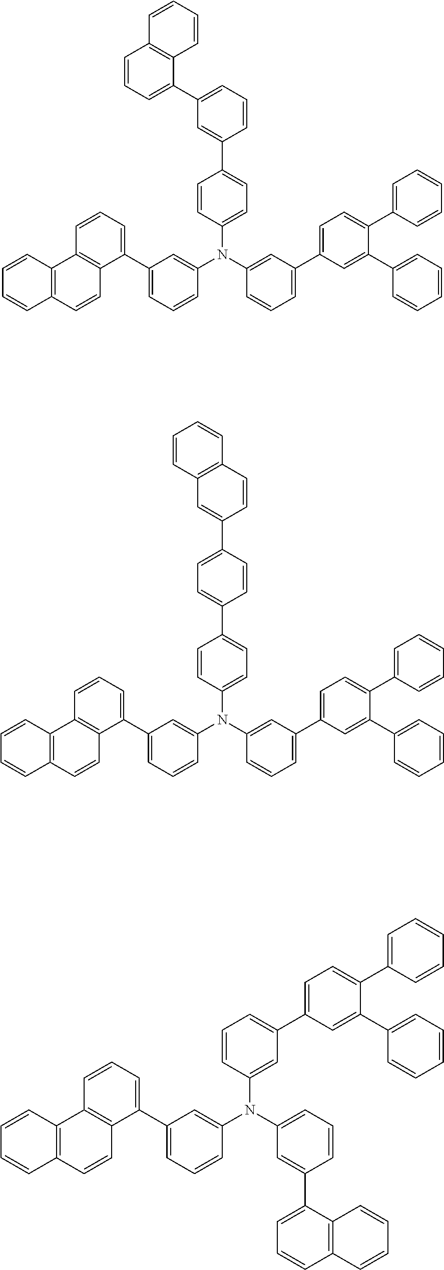 [SMILES: c1ccc(-c2ccc(-c3cccc(N(c4ccc(-c5cccc(-c6cccc7ccccc67)c5)cc4)c4cccc(-c5cccc6c5ccc5ccccc56)c4)c3)cc2-c2ccccc2)cc1 ; c1ccc(-c2ccc(-c3cccc(N(c4ccc(-c5ccc(-c6ccc7ccccc7c6)cc5)cc4)c4cccc(-c5cccc6c5ccc5ccccc56)c4)c3)cc2-c2ccccc2)cc1 ; c1ccc(-c2ccc(-c3cccc(N(c4cccc(-c5cccc6ccccc56)c4)c4cccc(-c5cccc6c5ccc5ccccc56)c4)c3)cc2-c2ccccc2)cc1]

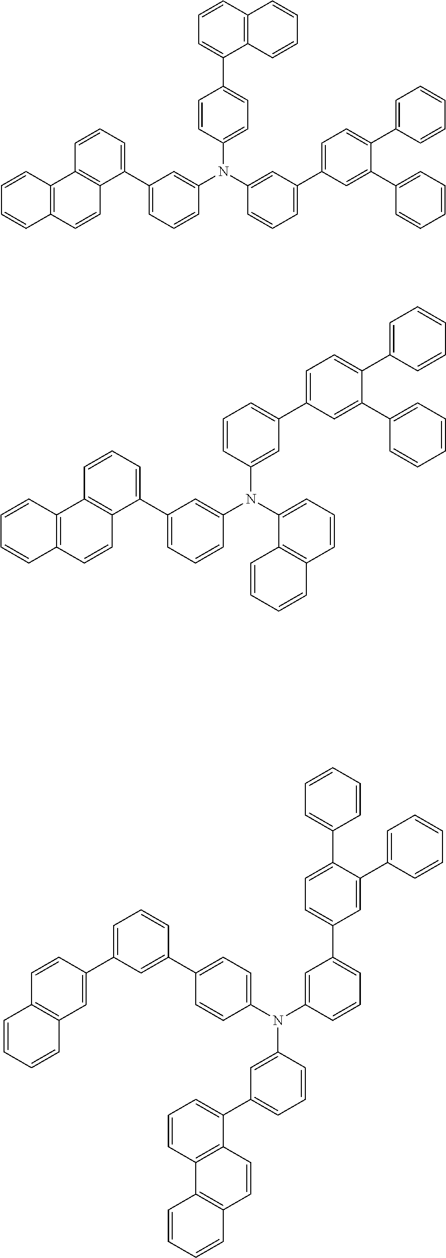 [SMILES: c1ccc(-c2ccc(-c3cccc(N(c4ccc(-c5cccc6ccccc56)cc4)c4cccc(-c5cccc6c5ccc5ccccc56)c4)c3)cc2-c2ccccc2)cc1 ; c1ccc(-c2ccc(-c3cccc(N(c4cccc(-c5cccc6c5ccc5ccccc56)c4)c4cccc5ccccc45)c3)cc2-c2ccccc2)cc1 ; c1ccc(-c2ccc(-c3cccc(N(c4ccc(-c5cccc(-c6ccc7ccccc7c6)c5)cc4)c4cccc(-c5cccc6c5ccc5ccccc56)c4)c3)cc2-c2ccccc2)cc1]

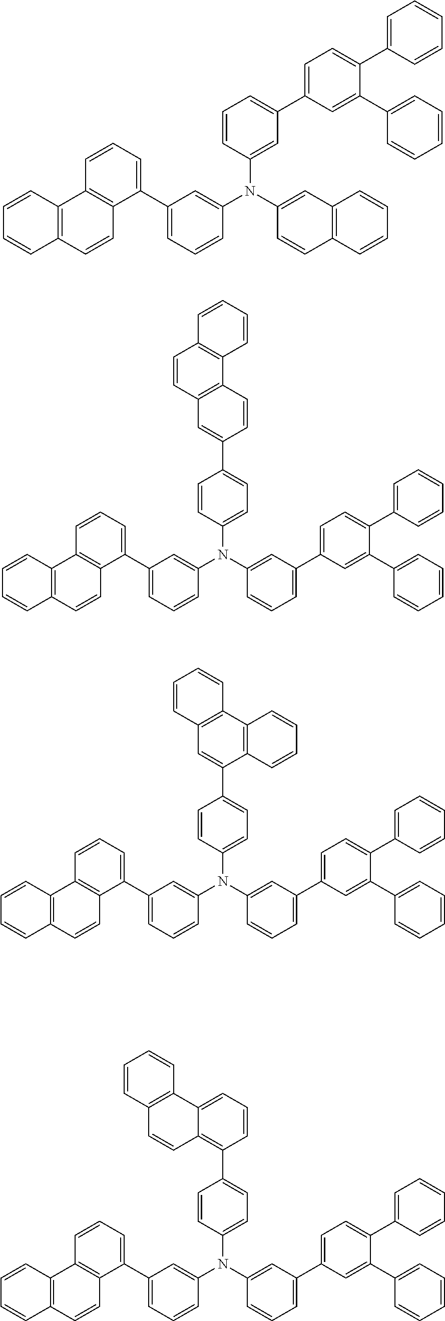 [SMILES: c1ccc(-c2ccc(-c3cccc(N(c4cccc(-c5cccc6c5ccc5ccccc56)c4)c4ccc5ccccc5c4)c3)cc2-c2ccccc2)cc1 ; c1ccc(-c2ccc(-c3cccc(N(c4ccc(-c5ccc6c(ccc7ccccc76)c5)cc4)c4cccc(-c5cccc6c5ccc5ccccc56)c4)c3)cc2-c2ccccc2)cc1 ; c1ccc(-c2ccc(-c3cccc(N(c4ccc(-c5cc6ccccc6c6ccccc56)cc4)c4cccc(-c5cccc6c5ccc5ccccc56)c4)c3)cc2-c2ccccc2)cc1 ; c1ccc(-c2ccc(-c3cccc(N(c4ccc(-c5cccc6c5ccc5ccccc56)cc4)c4cccc(-c5cccc6c5ccc5ccccc56)c4)c3)cc2-c2ccccc2)cc1]

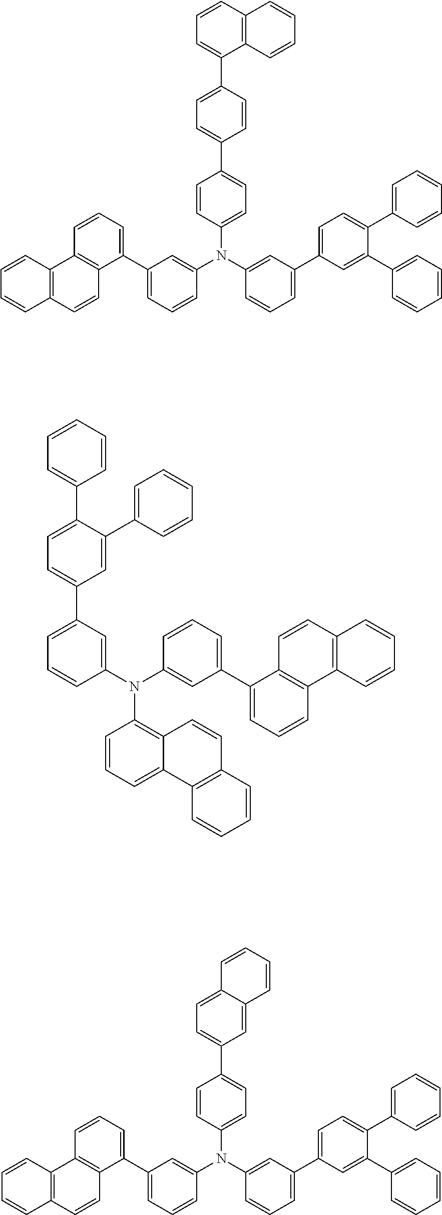 [SMILES: c1ccc(-c2ccc(-c3cccc(N(c4ccc(-c5ccc(-c6cccc7ccccc67)cc5)cc4)c4cccc(-c5cccc6c5ccc5ccccc56)c4)c3)cc2-c2ccccc2)cc1 ; c1ccc(-c2ccc(-c3cccc(N(c4cccc(-c5cccc6c5ccc5ccccc56)c4)c4cccc5c4ccc4ccccc45)c3)cc2-c2ccccc2)cc1 ; c1ccc(-c2ccc(-c3cccc(N(c4ccc(-c5ccc6ccccc6c5)cc4)c4cccc(-c5cccc6c5ccc5ccccc56)c4)c3)cc2-c2ccccc2)cc1]

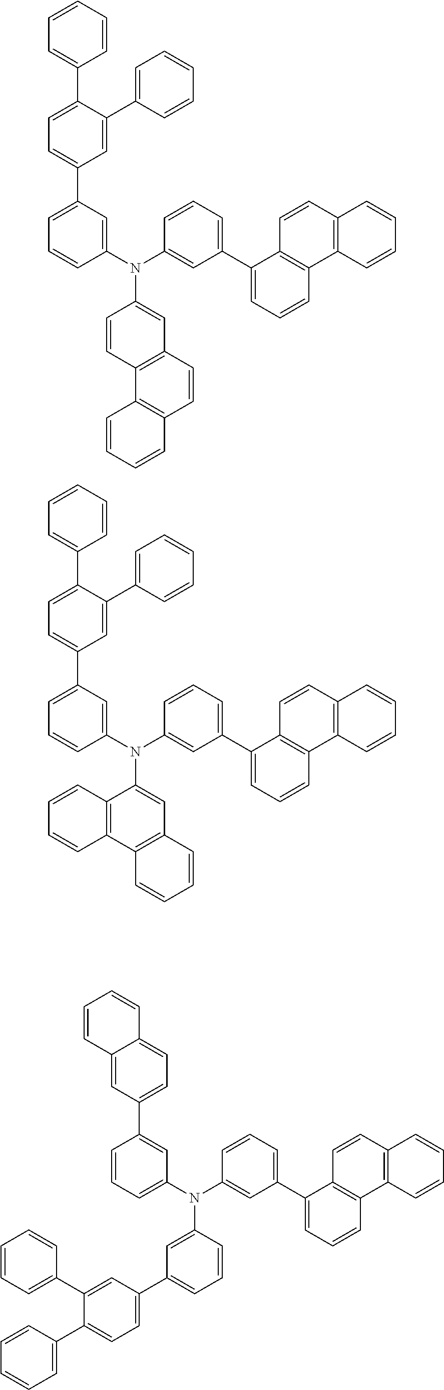

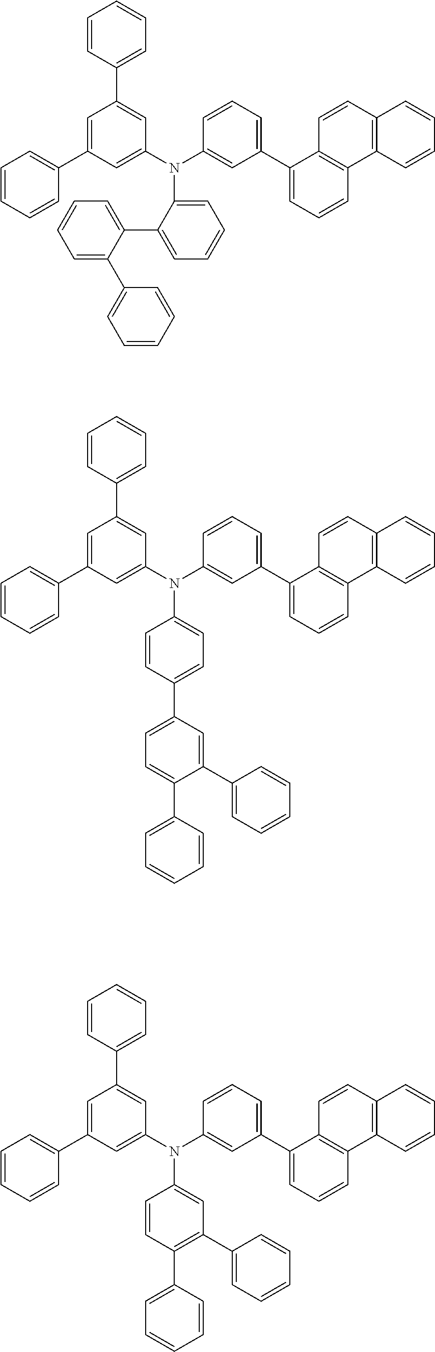 [SMILES: c1ccc(-c2cc(-c3ccccc3)cc(N(c3cccc(-c4cccc5c4ccc4ccccc45)c3)c3ccccc3-c3ccccc3-c3ccccc3)c2)cc1 ; c1ccc(-c2cc(-c3ccccc3)cc(N(c3ccc(-c4ccc(-c5ccccc5)c(-c5ccccc5)c4)cc3)c3cccc(-c4cccc5c4ccc4ccccc45)c3)c2)cc1 ; c1ccc(-c2cc(-c3ccccc3)cc(N(c3cccc(-c4cccc5c4ccc4ccccc45)c3)c3ccc(-c4ccccc4)c(-c4ccccc4)c3)c2)cc1]

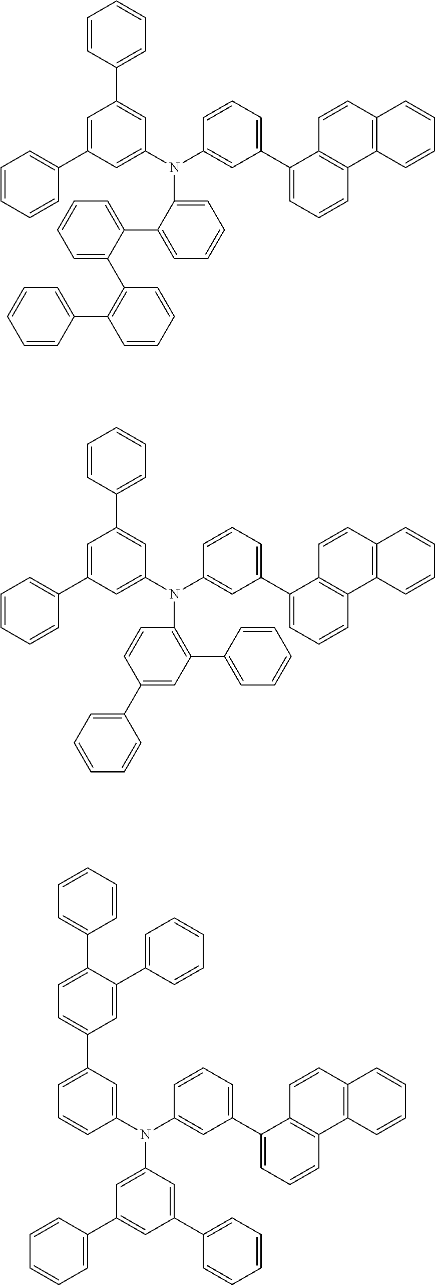 [SMILES: c1ccc(-c2cc(-c3ccccc3)cc(N(c3cccc(-c4cccc5c4ccc4ccccc45)c3)c3ccccc3-c3ccccc3-c3ccccc3-c3ccccc3)c2)cc1 ; c1ccc(-c2cc(-c3ccccc3)cc(N(c3cccc(-c4cccc5c4ccc4ccccc45)c3)c3ccc(-c4ccccc4)cc3-c3ccccc3)c2)cc1 ; c1ccc(-c2cc(-c3ccccc3)cc(N(c3cccc(-c4ccc(-c5ccccc5)c(-c5ccccc5)c4)c3)c3cccc(-c4cccc5c4ccc4ccccc45)c3)c2)cc1]

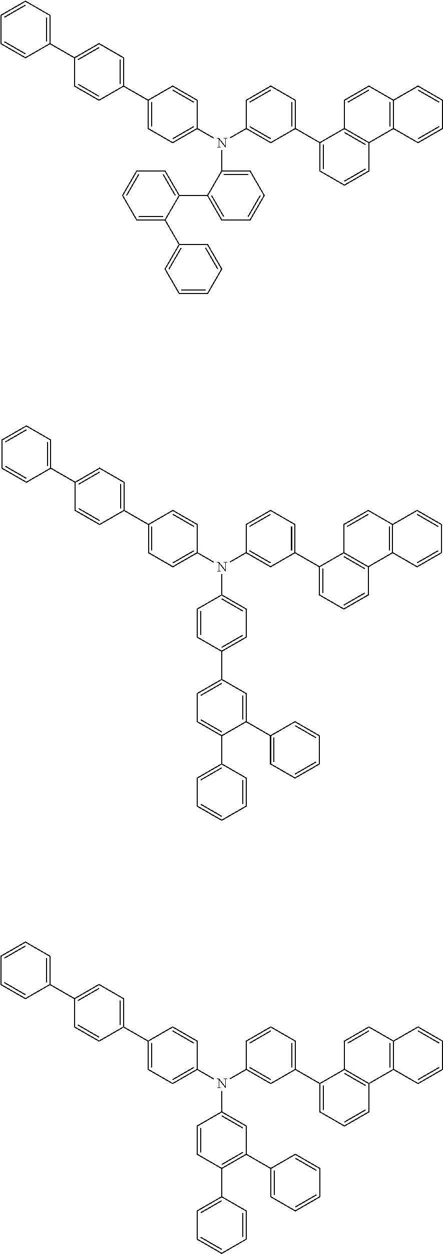 [SMILES: c1ccc(-c2ccc(-c3ccc(N(c4cccc(-c5cccc6c5ccc5ccccc56)c4)c4ccccc4-c4ccccc4-c4ccccc4)cc3)cc2)cc1 ; c1ccc(-c2ccc(-c3ccc(N(c4ccc(-c5ccc(-c6ccccc6)c(-c6ccccc6)c5)cc4)c4cccc(-c5cccc6c5ccc5ccccc56)c4)cc3)cc2)cc1 ; c1ccc(-c2ccc(-c3ccc(N(c4cccc(-c5cccc6c5ccc5ccccc56)c4)c4ccc(-c5ccccc5)c(-c5ccccc5)c4)cc3)cc2)cc1]

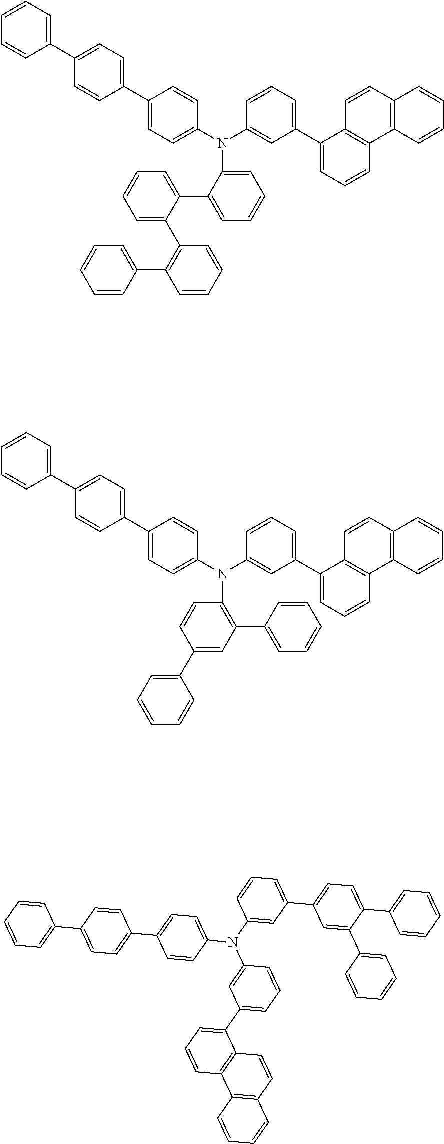 [SMILES: c1ccc(-c2ccc(-c3ccc(N(c4cccc(-c5cccc6c5ccc5ccccc56)c4)c4ccccc4-c4ccccc4-c4ccccc4-c4ccccc4)cc3)cc2)cc1 ; c1ccc(-c2ccc(-c3ccc(N(c4cccc(-c5cccc6c5ccc5ccccc56)c4)c4ccc(-c5ccccc5)cc4-c4ccccc4)cc3)cc2)cc1 ; c1ccc(-c2ccc(-c3ccc(N(c4cccc(-c5ccc(-c6ccccc6)c(-c6ccccc6)c5)c4)c4cccc(-c5cccc6c5ccc5ccccc56)c4)cc3)cc2)cc1]

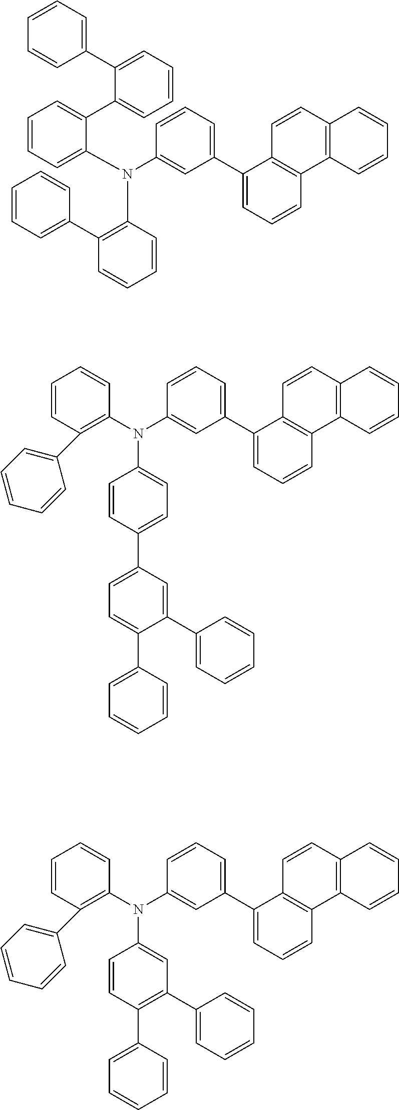 [SMILES: c1ccc(-c2ccccc2-c2ccccc2N(c2cccc(-c3cccc4c3ccc3ccccc34)c2)c2ccccc2-c2ccccc2)cc1 ; c1ccc(-c2ccc(-c3ccc(N(c4cccc(-c5cccc6c5ccc5ccccc56)c4)c4ccccc4-c4ccccc4)cc3)cc2-c2ccccc2)cc1 ; c1ccc(-c2ccc(N(c3cccc(-c4cccc5c4ccc4ccccc45)c3)c3ccccc3-c3ccccc3)cc2-c2ccccc2)cc1]

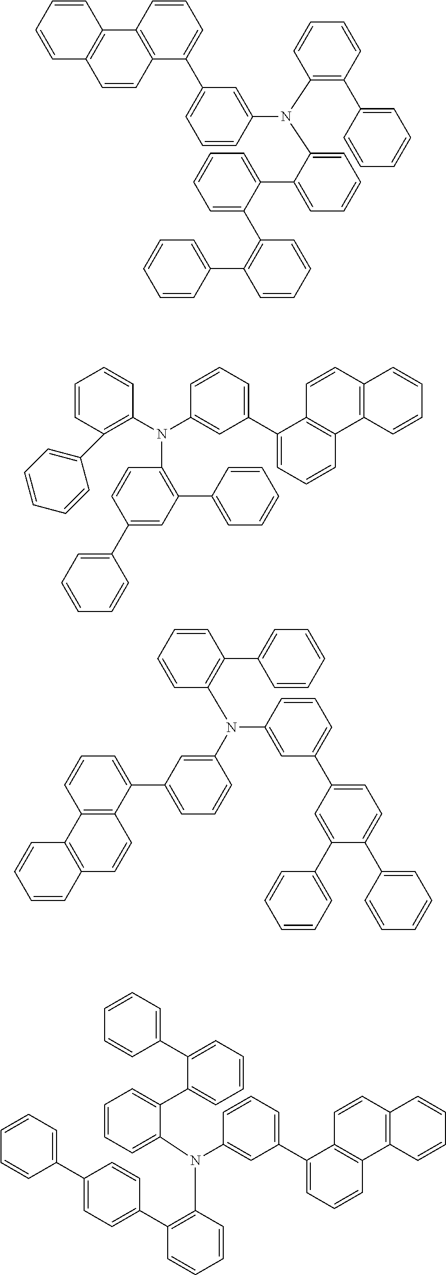 [SMILES: c1ccc(-c2ccccc2-c2ccccc2-c2ccccc2N(c2cccc(-c3cccc4c3ccc3ccccc34)c2)c2ccccc2-c2ccccc2)cc1 ; c1ccc(-c2ccc(N(c3cccc(-c4cccc5c4ccc4ccccc45)c3)c3ccccc3-c3ccccc3)c(-c3ccccc3)c2)cc1 ; c1ccc(-c2ccc(-c3cccc(N(c4cccc(-c5cccc6c5ccc5ccccc56)c4)c4ccccc4-c4ccccc4)c3)cc2-c2ccccc2)cc1 ; c1ccc(-c2ccc(-c3ccccc3N(c3cccc(-c4cccc5c4ccc4ccccc45)c3)c3ccccc3-c3ccccc3-c3ccccc3)cc2)cc1]

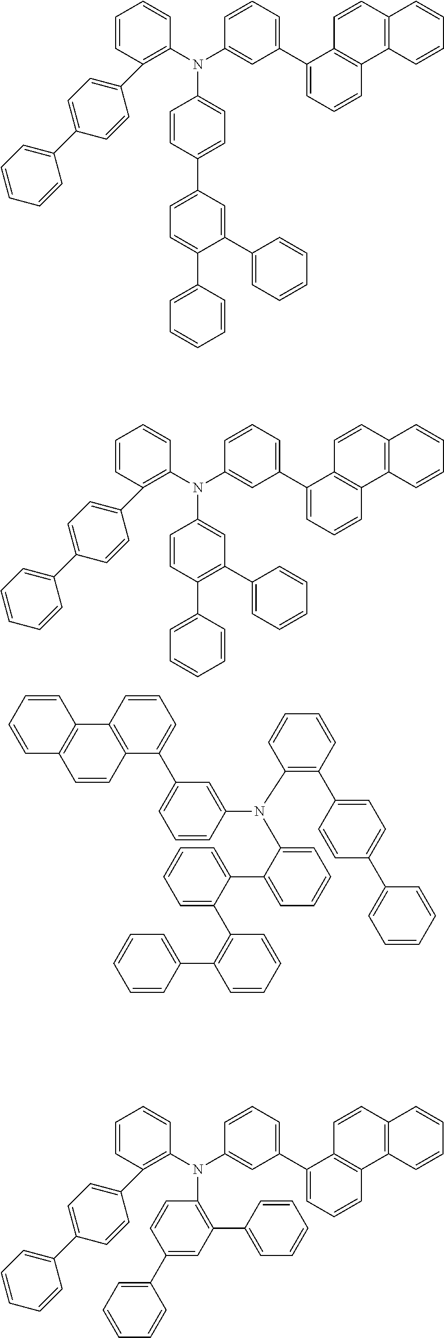

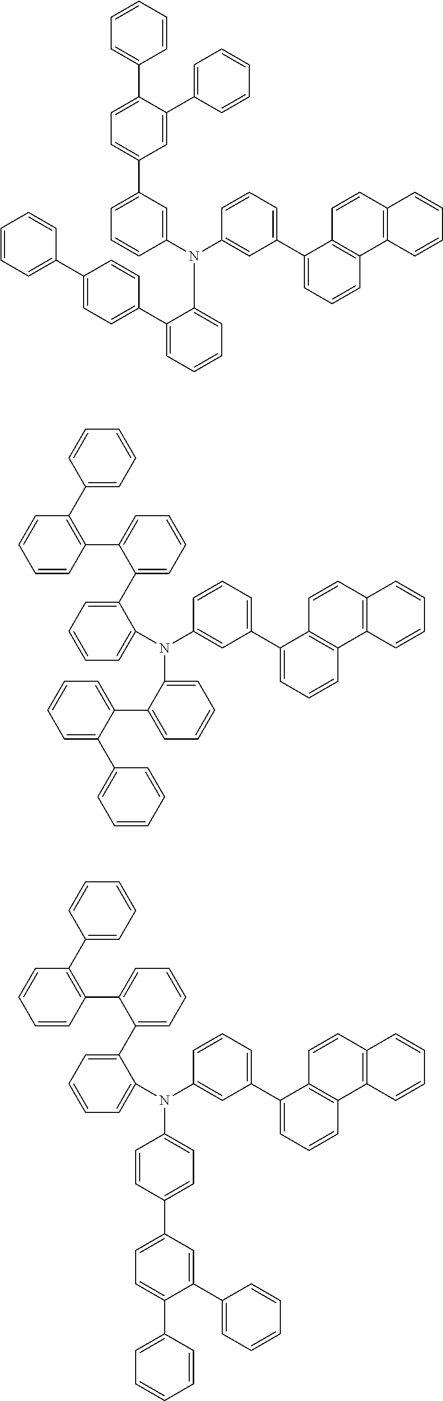 [SMILES: c1ccc(-c2ccc(-c3ccccc3N(c3cccc(-c4ccc(-c5ccccc5)c(-c5ccccc5)c4)c3)c3cccc(-c4cccc5c4ccc4ccccc45)c3)cc2)cc1 ; c1ccc(-c2ccccc2-c2ccccc2-c2ccccc2N(c2cccc(-c3cccc4c3ccc3ccccc34)c2)c2ccccc2-c2ccccc2-c2ccccc2)cc1 ; c1ccc(-c2ccc(-c3ccc(N(c4cccc(-c5cccc6c5ccc5ccccc56)c4)c4ccccc4-c4ccccc4-c4ccccc4-c4ccccc4)cc3)cc2-c2ccccc2)cc1]

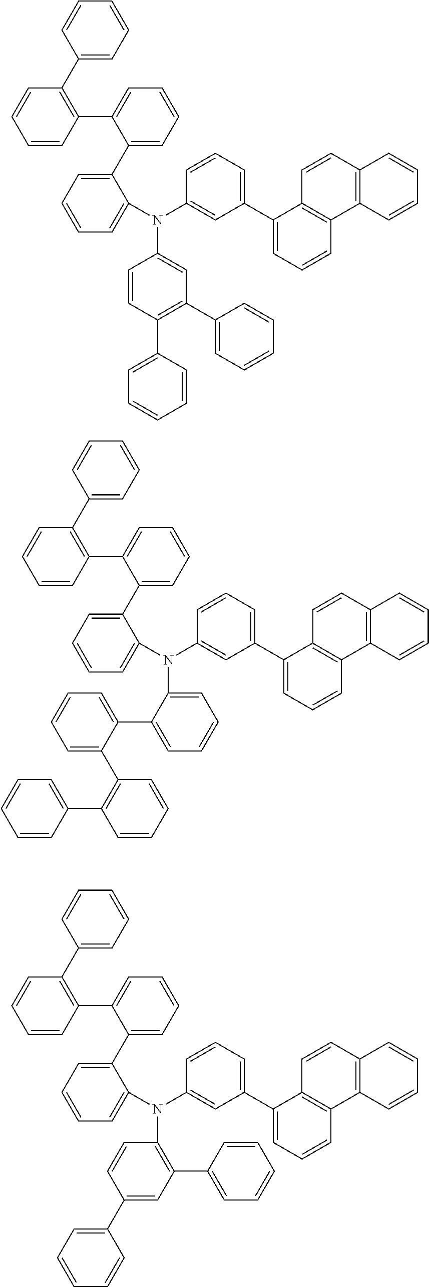

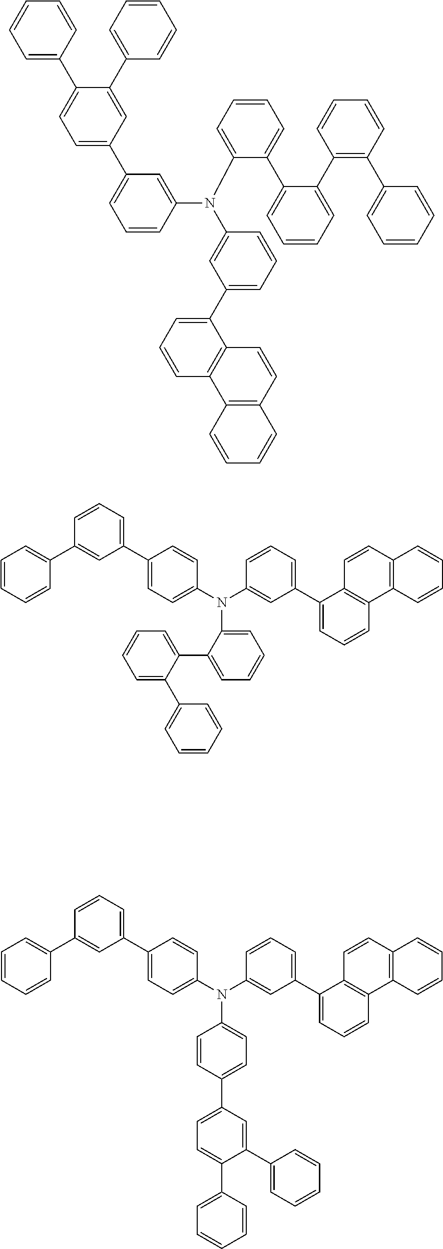 [SMILES: c1ccc(-c2ccc(-c3cccc(N(c4cccc(-c5cccc6c5ccc5ccccc56)c4)c4ccccc4-c4ccccc4-c4ccccc4-c4ccccc4)c3)cc2-c2ccccc2)cc1 ; c1ccc(-c2cccc(-c3ccc(N(c4cccc(-c5cccc6c5ccc5ccccc56)c4)c4ccccc4-c4ccccc4-c4ccccc4)cc3)c2)cc1 ; c1ccc(-c2cccc(-c3ccc(N(c4ccc(-c5ccc(-c6ccccc6)c(-c6ccccc6)c5)cc4)c4cccc(-c5cccc6c5ccc5ccccc56)c4)cc3)c2)cc1]

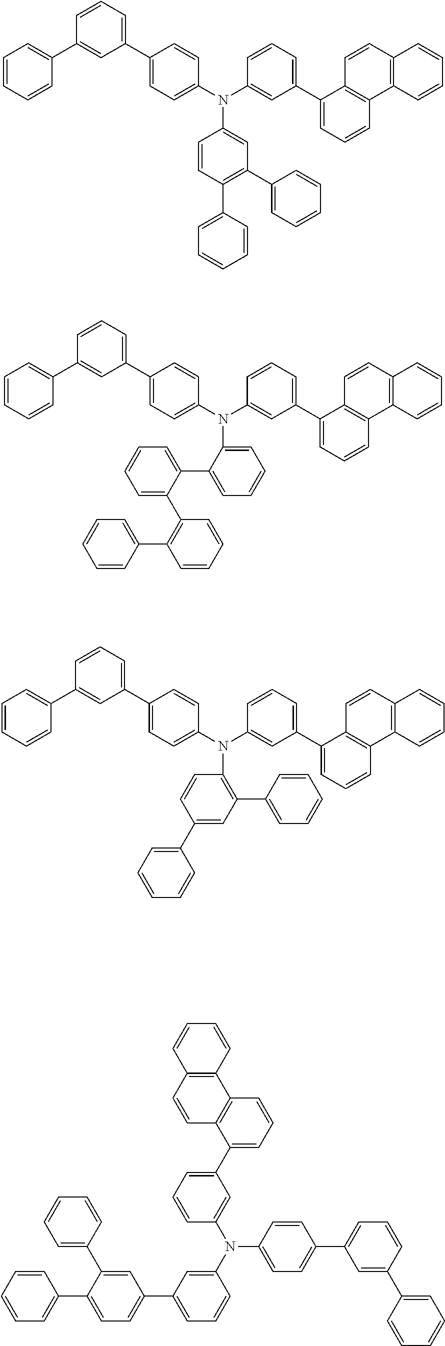 [SMILES: c1ccc(-c2cccc(-c3ccc(N(c4cccc(-c5cccc6c5ccc5ccccc56)c4)c4ccc(-c5ccccc5)c(-c5ccccc5)c4)cc3)c2)cc1 ; c1ccc(-c2cccc(-c3ccc(N(c4cccc(-c5cccc6c5ccc5ccccc56)c4)c4ccccc4-c4ccccc4-c4ccccc4-c4ccccc4)cc3)c2)cc1 ; c1ccc(-c2cccc(-c3ccc(N(c4cccc(-c5cccc6c5ccc5ccccc56)c4)c4ccc(-c5ccccc5)cc4-c4ccccc4)cc3)c2)cc1 ; c1ccc(-c2cccc(-c3ccc(N(c4cccc(-c5ccc(-c6ccccc6)c(-c6ccccc6)c5)c4)c4cccc(-c5cccc6c5ccc5ccccc56)c4)cc3)c2)cc1]

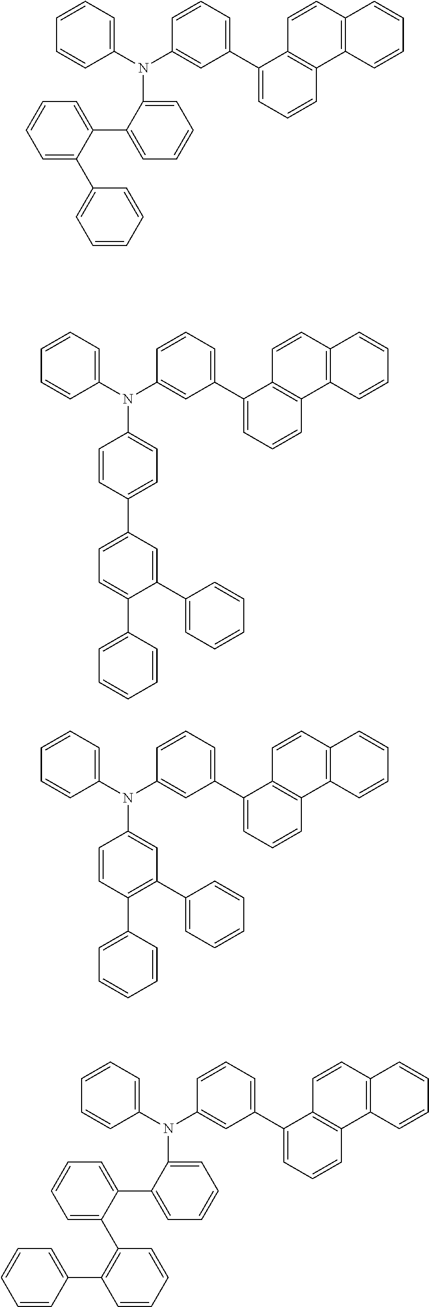 [SMILES: c1ccc(-c2ccccc2-c2ccccc2N(c2ccccc2)c2cccc(-c3cccc4c3ccc3ccccc34)c2)cc1 ; c1ccc(-c2ccc(-c3ccc(N(c4ccccc4)c4cccc(-c5cccc6c5ccc5ccccc56)c4)cc3)cc2-c2ccccc2)cc1 ; c1ccc(-c2ccc(N(c3ccccc3)c3cccc(-c4cccc5c4ccc4ccccc45)c3)cc2-c2ccccc2)cc1 ; c1ccc(-c2ccccc2-c2ccccc2-c2ccccc2N(c2ccccc2)c2cccc(-c3cccc4c3ccc3ccccc34)c2)cc1]

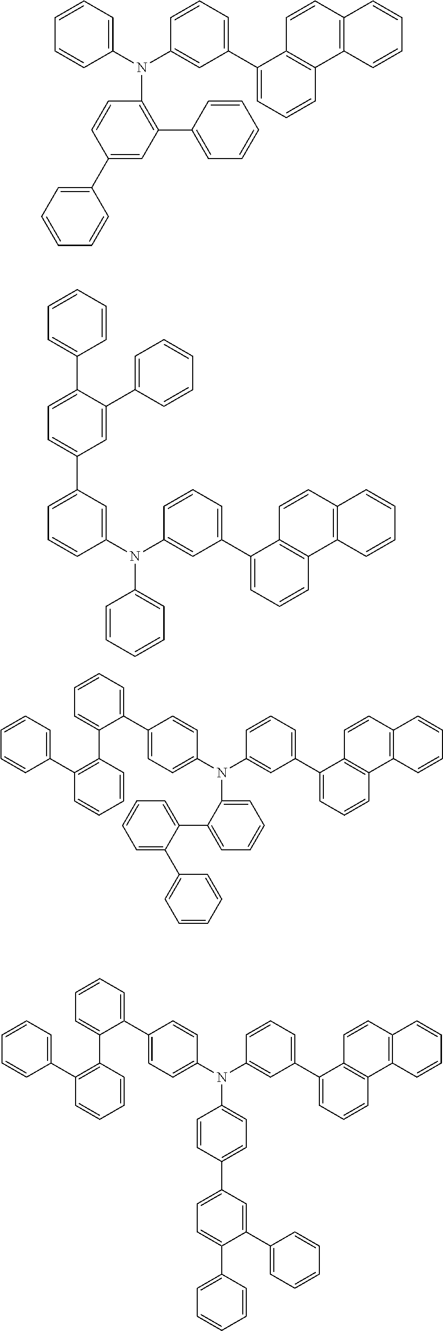

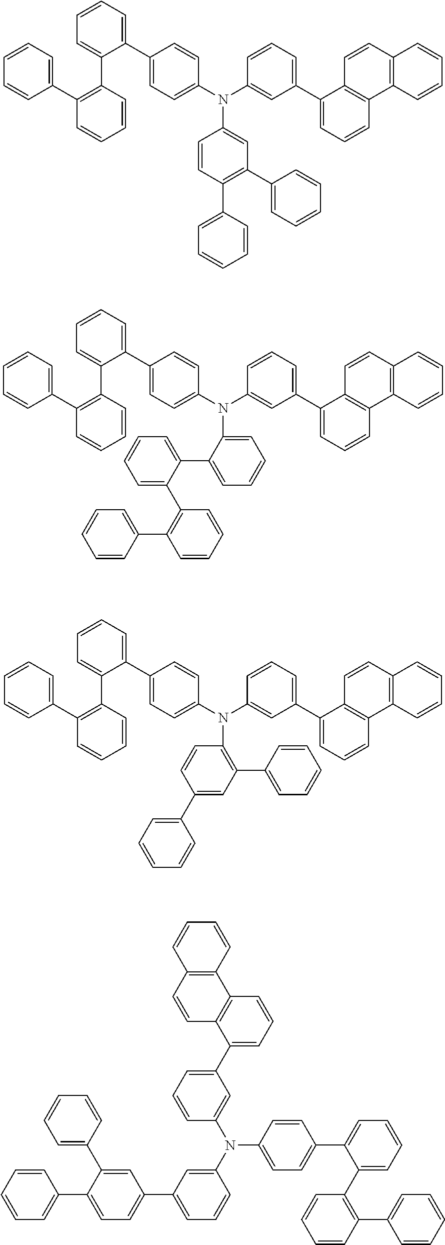 [SMILES: c1ccc(-c2ccc(N(c3ccc(-c4ccccc4-c4ccccc4-c4ccccc4)cc3)c3cccc(-c4cccc5c4ccc4ccccc45)c3)cc2-c2ccccc2)cc1 ; c1ccc(-c2ccccc2-c2ccccc2-c2ccc(N(c3cccc(-c4cccc5c4ccc4ccccc45)c3)c3ccccc3-c3ccccc3-c3ccccc3-c3ccccc3)cc2)cc1 ; c1ccc(-c2ccc(N(c3ccc(-c4ccccc4-c4ccccc4-c4ccccc4)cc3)c3cccc(-c4cccc5c4ccc4ccccc45)c3)c(-c3ccccc3)c2)cc1 ; c1ccc(-c2ccc(-c3cccc(N(c4ccc(-c5ccccc5-c5ccccc5-c5ccccc5)cc4)c4cccc(-c5cccc6c5ccc5ccccc56)c4)c3)cc2-c2ccccc2)cc1]

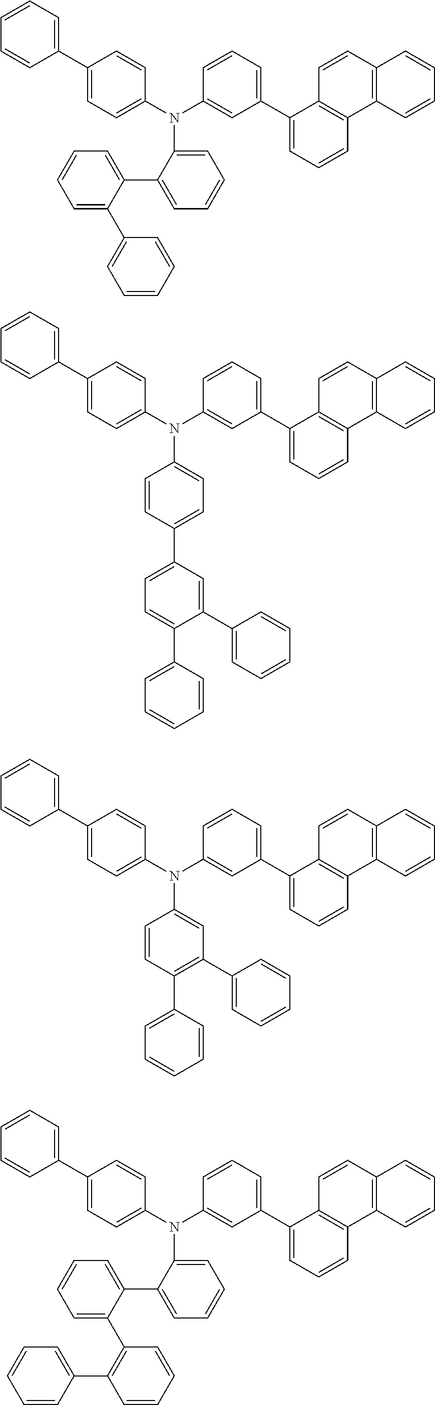 [SMILES: c1ccc(-c2ccc(N(c3cccc(-c4cccc5c4ccc4ccccc45)c3)c3ccccc3-c3ccccc3-c3ccccc3)cc2)cc1 ; c1ccc(-c2ccc(N(c3ccc(-c4ccc(-c5ccccc5)c(-c5ccccc5)c4)cc3)c3cccc(-c4cccc5c4ccc4ccccc45)c3)cc2)cc1 ; c1ccc(-c2ccc(N(c3cccc(-c4cccc5c4ccc4ccccc45)c3)c3ccc(-c4ccccc4)c(-c4ccccc4)c3)cc2)cc1 ; c1ccc(-c2ccc(N(c3cccc(-c4cccc5c4ccc4ccccc45)c3)c3ccccc3-c3ccccc3-c3ccccc3-c3ccccc3)cc2)cc1]

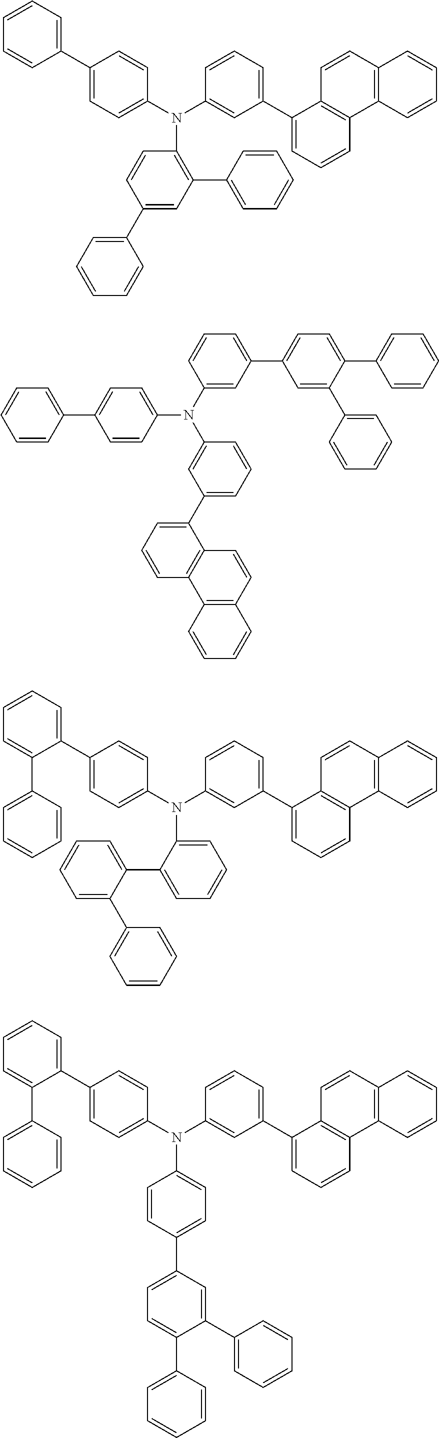 [SMILES: c1ccc(-c2ccc(N(c3cccc(-c4cccc5c4ccc4ccccc45)c3)c3ccc(-c4ccccc4)cc3-c3ccccc3)cc2)cc1 ; c1ccc(-c2ccc(N(c3cccc(-c4ccc(-c5ccccc5)c(-c5ccccc5)c4)c3)c3cccc(-c4cccc5c4ccc4ccccc45)c3)cc2)cc1 ; c1ccc(-c2ccccc2-c2ccc(N(c3cccc(-c4cccc5c4ccc4ccccc45)c3)c3ccccc3-c3ccccc3-c3ccccc3)cc2)cc1 ; c1ccc(-c2ccccc2-c2ccc(N(c3ccc(-c4ccc(-c5ccccc5)c(-c5ccccc5)c4)cc3)c3cccc(-c4cccc5c4ccc4ccccc45)c3)cc2)cc1]

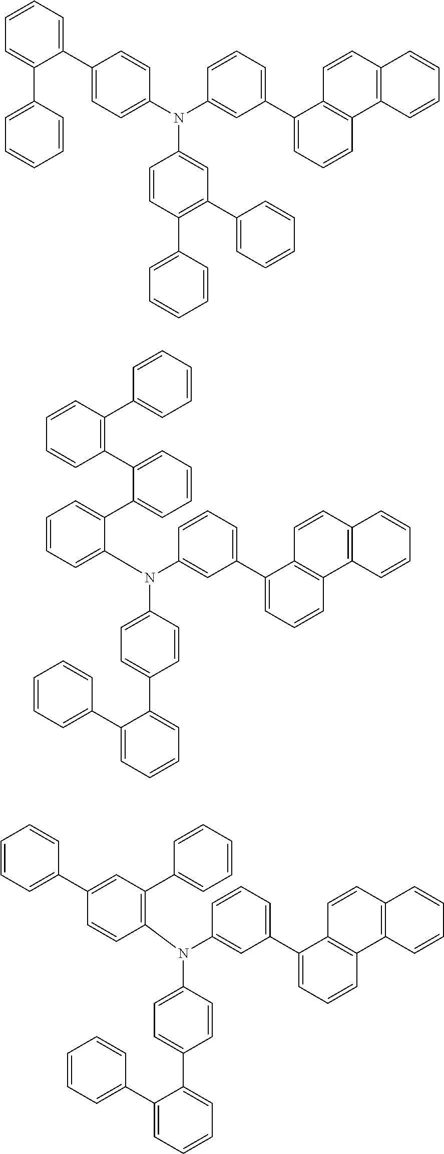

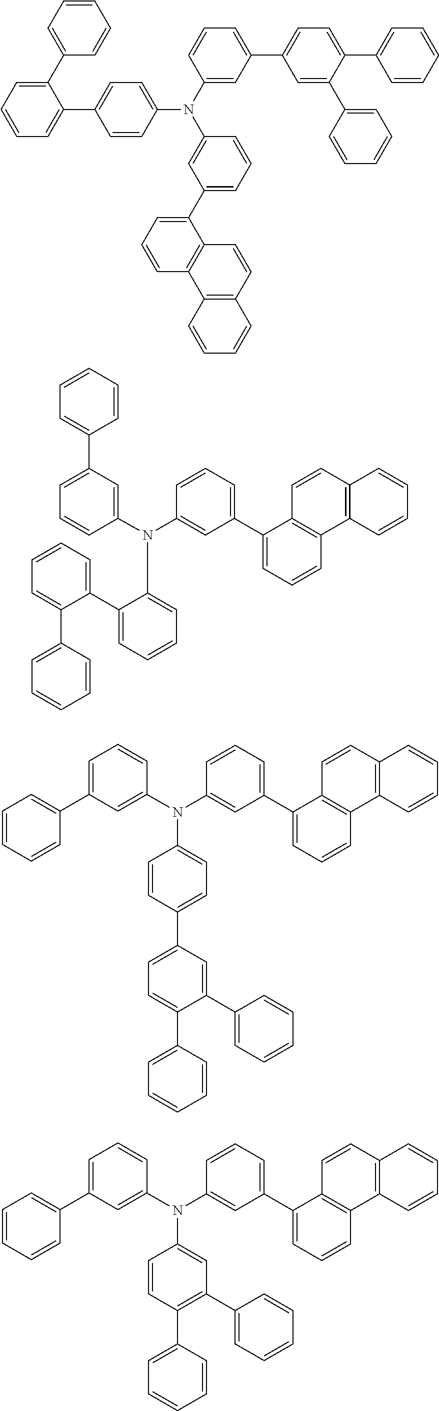 [SMILES: c1ccc(-c2ccccc2-c2ccc(N(c3cccc(-c4ccc(-c5ccccc5)c(-c5ccccc5)c4)c3)c3cccc(-c4cccc5c4ccc4ccccc45)c3)cc2)cc1 ; c1ccc(-c2cccc(N(c3cccc(-c4cccc5c4ccc4ccccc45)c3)c3ccccc3-c3ccccc3-c3ccccc3)c2)cc1 ; c1ccc(-c2cccc(N(c3ccc(-c4ccc(-c5ccccc5)c(-c5ccccc5)c4)cc3)c3cccc(-c4cccc5c4ccc4ccccc45)c3)c2)cc1 ; c1ccc(-c2cccc(N(c3cccc(-c4cccc5c4ccc4ccccc45)c3)c3ccc(-c4ccccc4)c(-c4ccccc4)c3)c2)cc1]

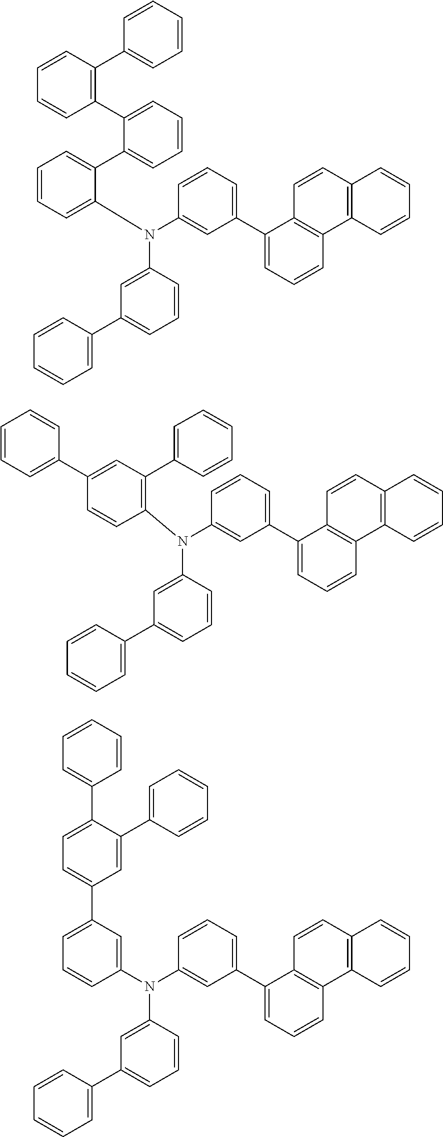

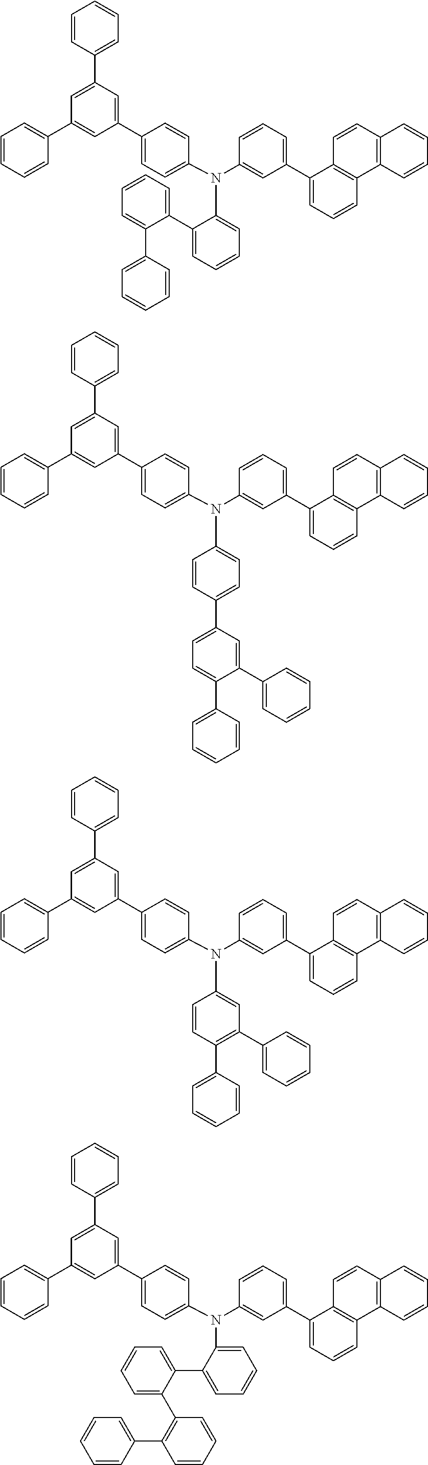 [SMILES: c1ccc(-c2cc(-c3ccccc3)cc(-c3ccc(N(c4cccc(-c5cccc6c5ccc5ccccc56)c4)c4ccccc4-c4ccccc4-c4ccccc4)cc3)c2)cc1 ; c1ccc(-c2cc(-c3ccccc3)cc(-c3ccc(N(c4ccc(-c5ccc(-c6ccccc6)c(-c6ccccc6)c5)cc4)c4cccc(-c5cccc6c5ccc5ccccc56)c4)cc3)c2)cc1 ; c1ccc(-c2cc(-c3ccccc3)cc(-c3ccc(N(c4cccc(-c5cccc6c5ccc5ccccc56)c4)c4ccc(-c5ccccc5)c(-c5ccccc5)c4)cc3)c2)cc1 ; c1ccc(-c2cc(-c3ccccc3)cc(-c3ccc(N(c4cccc(-c5cccc6c5ccc5ccccc56)c4)c4ccccc4-c4ccccc4-c4ccccc4-c4ccccc4)cc3)c2)cc1]

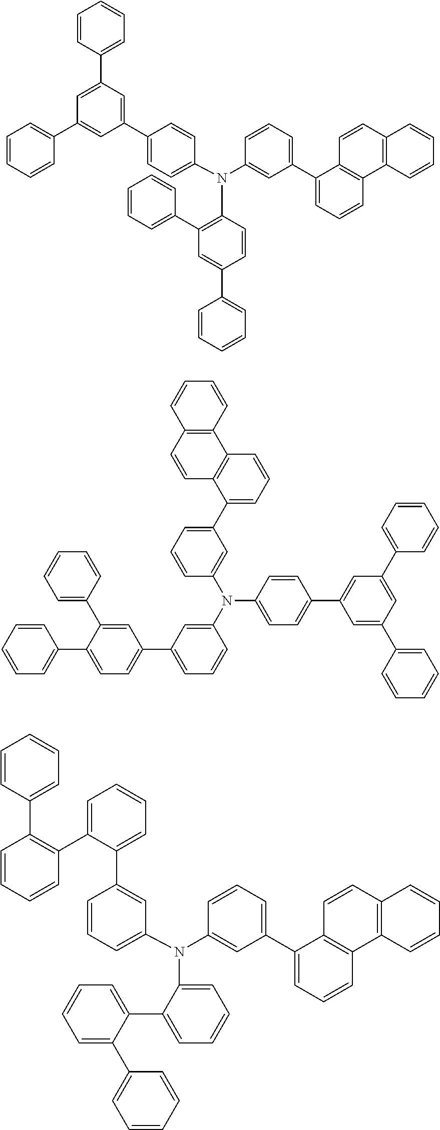

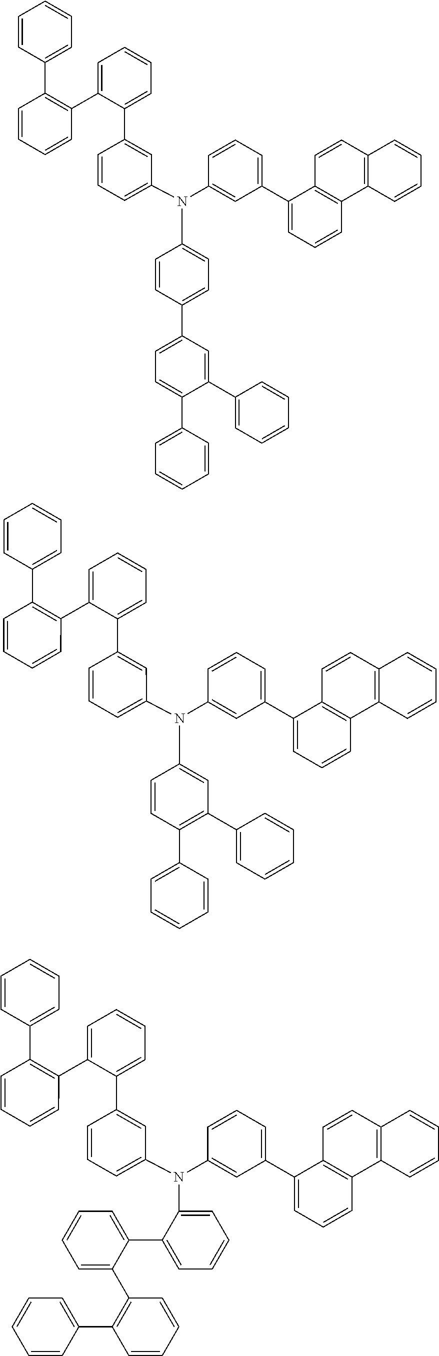 [SMILES: c1ccc(-c2ccc(-c3ccc(N(c4cccc(-c5ccccc5-c5ccccc5-c5ccccc5)c4)c4cccc(-c5cccc6c5ccc5ccccc56)c4)cc3)cc2-c2ccccc2)cc1 ; c1ccc(-c2ccc(N(c3cccc(-c4ccccc4-c4ccccc4-c4ccccc4)c3)c3cccc(-c4cccc5c4ccc4ccccc45)c3)cc2-c2ccccc2)cc1 ; c1ccc(-c2ccccc2-c2ccccc2-c2cccc(N(c3cccc(-c4cccc5c4ccc4ccccc45)c3)c3ccccc3-c3ccccc3-c3ccccc3-c3ccccc3)c2)cc1]

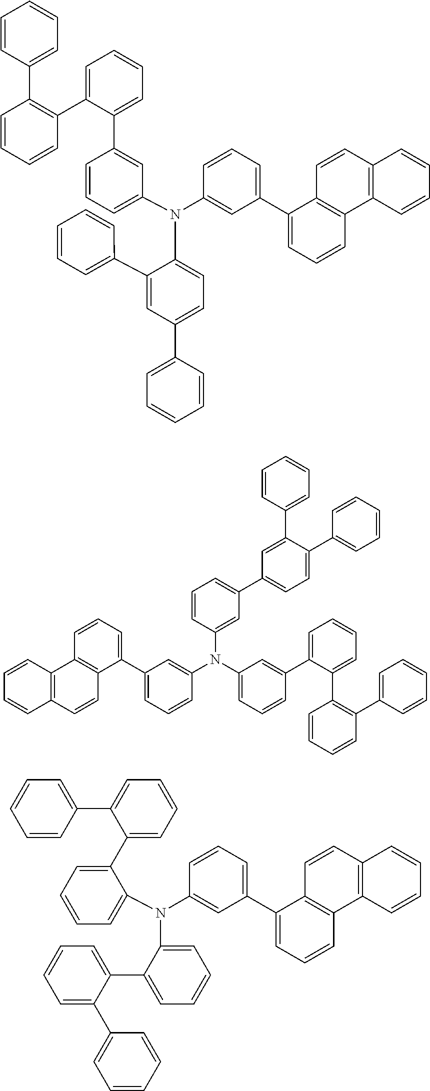 [SMILES: c1ccc(-c2ccc(N(c3cccc(-c4ccccc4-c4ccccc4-c4ccccc4)c3)c3cccc(-c4cccc5c4ccc4ccccc45)c3)c(-c3ccccc3)c2)cc1 ; c1ccc(-c2ccc(-c3cccc(N(c4cccc(-c5ccccc5-c5ccccc5-c5ccccc5)c4)c4cccc(-c5cccc6c5ccc5ccccc56)c4)c3)cc2-c2ccccc2)cc1 ; c1ccc(-c2ccccc2-c2ccccc2N(c2cccc(-c3cccc4c3ccc3ccccc34)c2)c2ccccc2-c2ccccc2-c2ccccc2)cc1]

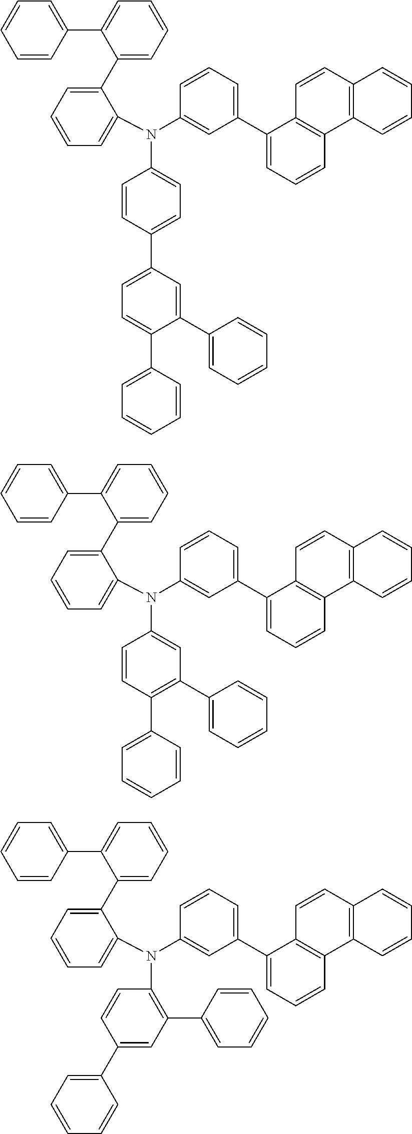

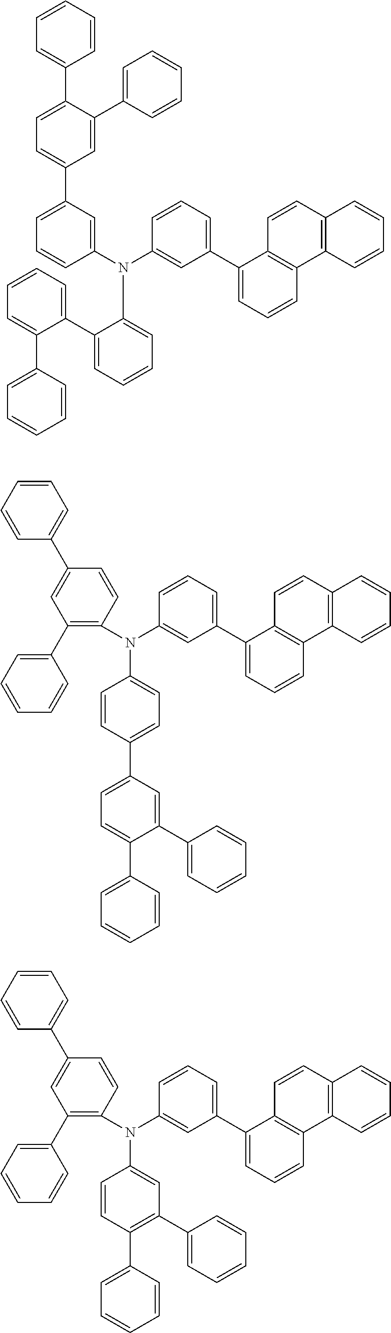 [SMILES: c1ccc(-c2ccc(-c3cccc(N(c4cccc(-c5cccc6c5ccc5ccccc56)c4)c4ccccc4-c4ccccc4-c4ccccc4)c3)cc2-c2ccccc2)cc1 ; c1ccc(-c2ccc(N(c3ccc(-c4ccc(-c5ccccc5)c(-c5ccccc5)c4)cc3)c3cccc(-c4cccc5c4ccc4ccccc45)c3)c(-c3ccccc3)c2)cc1 ; c1ccc(-c2ccc(N(c3cccc(-c4cccc5c4ccc4ccccc45)c3)c3ccc(-c4ccccc4)c(-c4ccccc4)c3)c(-c3ccccc3)c2)cc1]

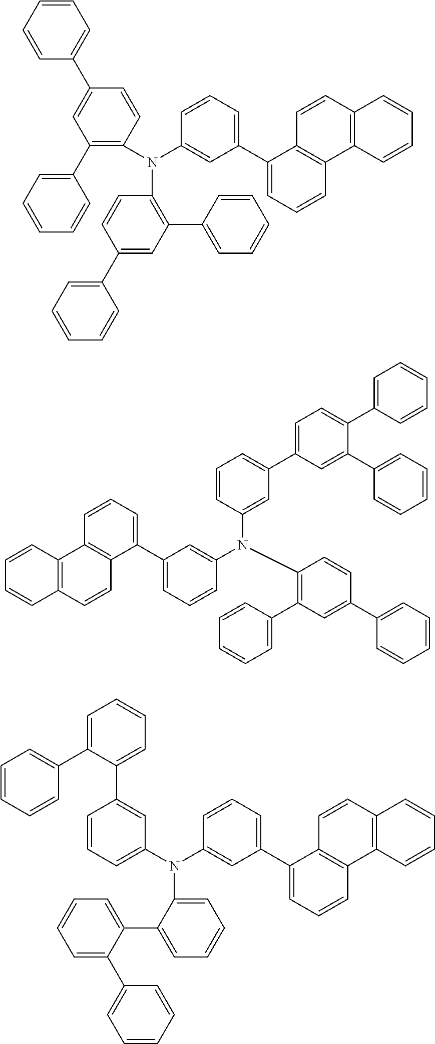 [SMILES: c1ccc(-c2ccc(N(c3cccc(-c4cccc5c4ccc4ccccc45)c3)c3ccc(-c4ccccc4)cc3-c3ccccc3)c(-c3ccccc3)c2)cc1 ; c1ccc(-c2ccc(N(c3cccc(-c4ccc(-c5ccccc5)c(-c5ccccc5)c4)c3)c3cccc(-c4cccc5c4ccc4ccccc45)c3)c(-c3ccccc3)c2)cc1 ; c1ccc(-c2ccccc2-c2cccc(N(c3cccc(-c4cccc5c4ccc4ccccc45)c3)c3ccccc3-c3ccccc3-c3ccccc3)c2)cc1]

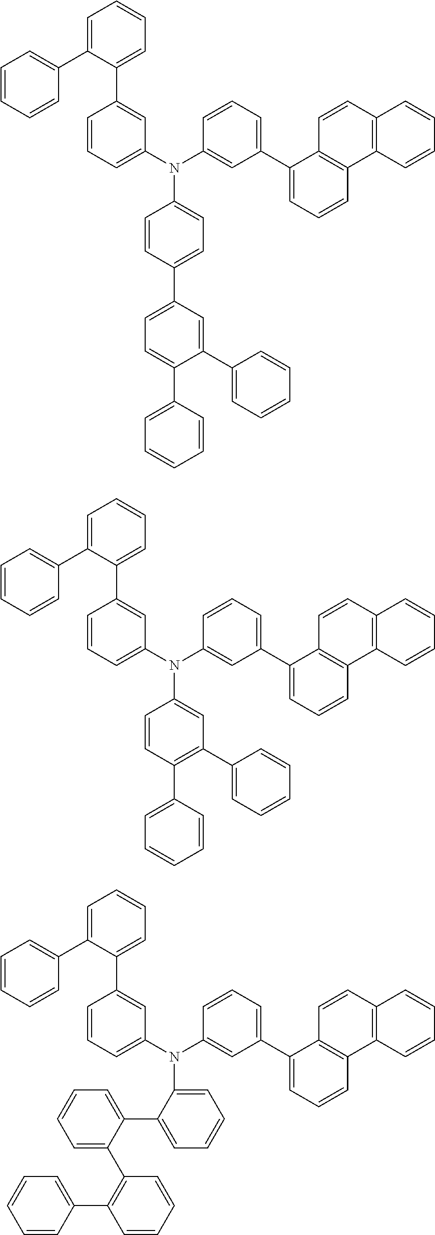 [SMILES: c1ccc(-c2ccccc2-c2cccc(N(c3ccc(-c4ccc(-c5ccccc5)c(-c5ccccc5)c4)cc3)c3cccc(-c4cccc5c4ccc4ccccc45)c3)c2)cc1 ; c1ccc(-c2ccccc2-c2cccc(N(c3cccc(-c4cccc5c4ccc4ccccc45)c3)c3ccc(-c4ccccc4)c(-c4ccccc4)c3)c2)cc1 ; c1ccc(-c2ccccc2-c2cccc(N(c3cccc(-c4cccc5c4ccc4ccccc45)c3)c3ccccc3-c3ccccc3-c3ccccc3-c3ccccc3)c2)cc1]

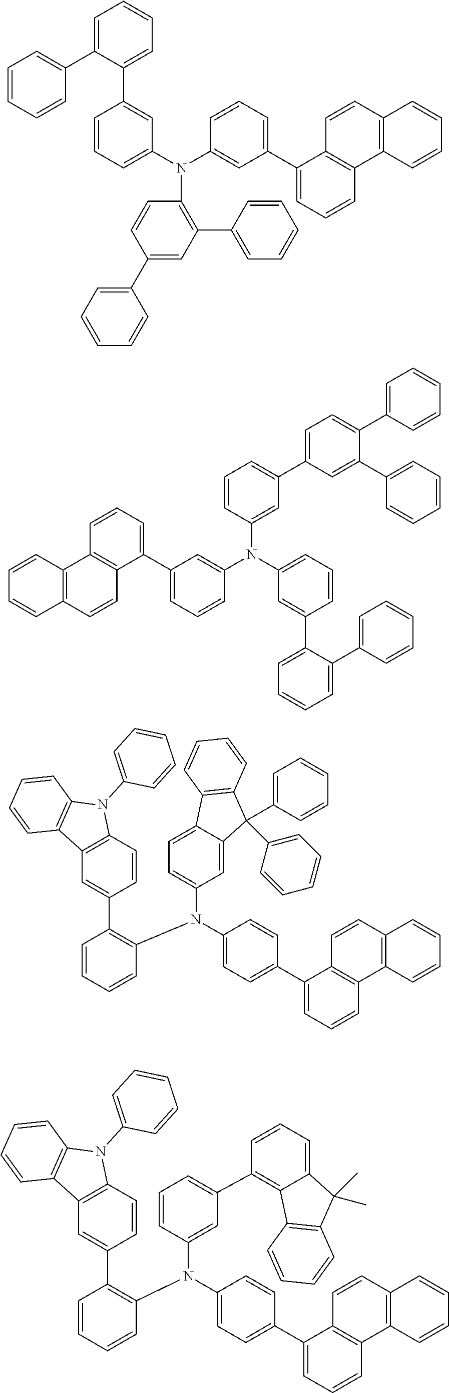 [SMILES: c1ccc(-c2ccc(N(c3cccc(-c4ccccc4-c4ccccc4)c3)c3cccc(-c4cccc5c4ccc4ccccc45)c3)c(-c3ccccc3)c2)cc1 ; c1ccc(-c2ccccc2-c2cccc(N(c3cccc(-c4ccc(-c5ccccc5)c(-c5ccccc5)c4)c3)c3cccc(-c4cccc5c4ccc4ccccc45)c3)c2)cc1 ; c1ccc(-n2c3ccccc3c3cc(-c4ccccc4N(c4ccc(-c5cccc6c5ccc5ccccc56)cc4)c4ccc5c(c4)C(c4ccccc4)(c4ccccc4)c4ccccc4-5)ccc32)cc1 ; CC1(C)c2ccccc2-c2c(-c3cccc(N(c4ccc(-c5cccc6c5ccc5ccccc56)cc4)c4ccccc4-c4ccc5c(c4)c4ccccc4n5-c4ccccc4)c3)cccc21]

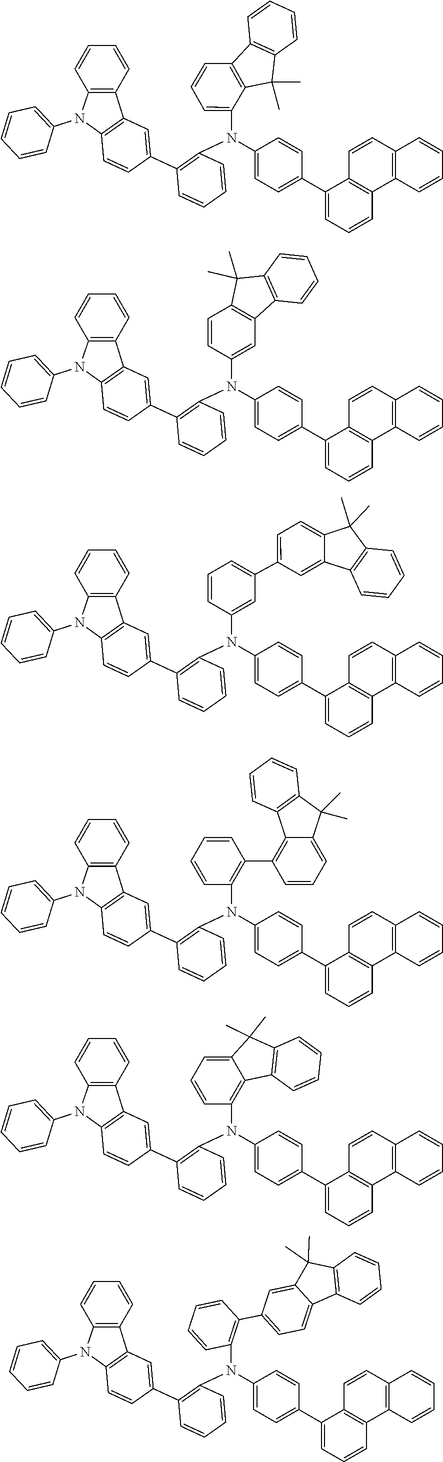

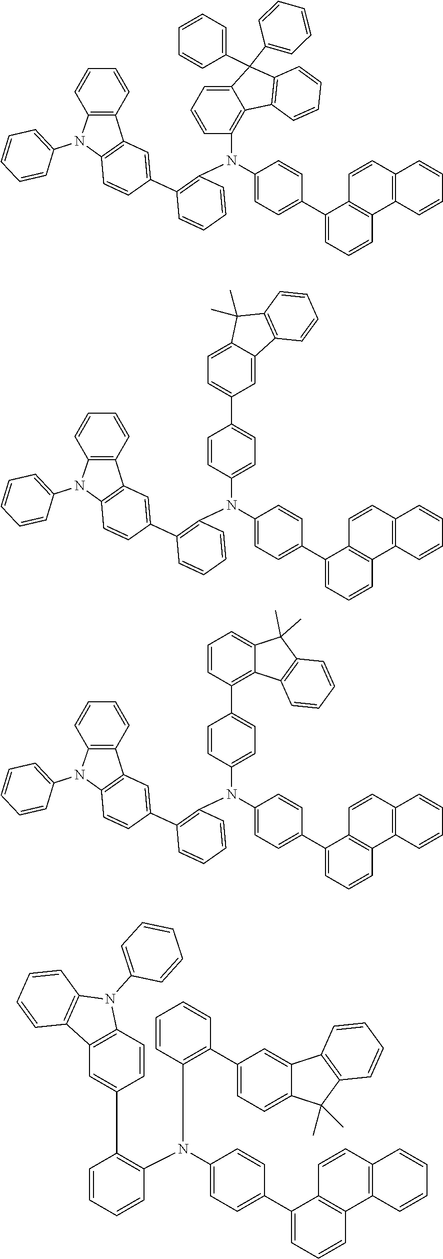 [SMILES: c1ccc(-n2c3ccccc3c3cc(-c4ccccc4N(c4ccc(-c5cccc6c5ccc5ccccc56)cc4)c4cccc5c4-c4ccccc4C5(c4ccccc4)c4ccccc4)ccc32)cc1 ; CC1(C)c2ccccc2-c2cc(-c3ccc(N(c4ccc(-c5cccc6c5ccc5ccccc56)cc4)c4ccccc4-c4ccc5c(c4)c4ccccc4n5-c4ccccc4)cc3)ccc21 ; CC1(C)c2ccccc2-c2c(-c3ccc(N(c4ccc(-c5cccc6c5ccc5ccccc56)cc4)c4ccccc4-c4ccc5c(c4)c4ccccc4n5-c4ccccc4)cc3)cccc21 ; CC1(C)c2ccccc2-c2cc(-c3ccccc3N(c3ccc(-c4cccc5c4ccc4ccccc45)cc3)c3ccccc3-c3ccc4c(c3)c3ccccc3n4-c3ccccc3)ccc21]

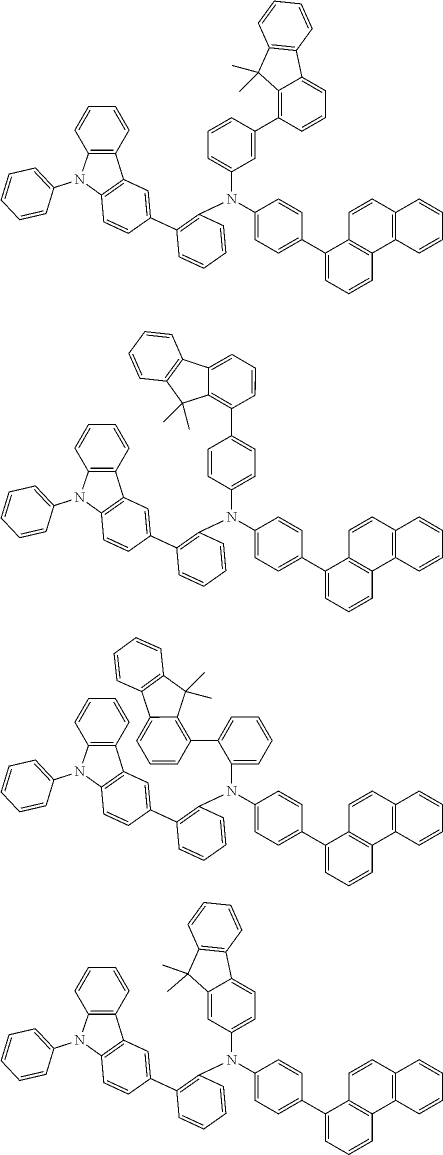 [SMILES: CC1(C)c2ccccc2-c2cccc(-c3cccc(N(c4ccc(-c5cccc6c5ccc5ccccc56)cc4)c4ccccc4-c4ccc5c(c4)c4ccccc4n5-c4ccccc4)c3)c21 ; CC1(C)c2ccccc2-c2cccc(-c3ccc(N(c4ccc(-c5cccc6c5ccc5ccccc56)cc4)c4ccccc4-c4ccc5c(c4)c4ccccc4n5-c4ccccc4)cc3)c21 ; CC1(C)c2ccccc2-c2cccc(-c3ccccc3N(c3ccc(-c4cccc5c4ccc4ccccc45)cc3)c3ccccc3-c3ccc4c(c3)c3ccccc3n4-c3ccccc3)c21 ; CC1(C)c2ccccc2-c2ccc(N(c3ccc(-c4cccc5c4ccc4ccccc45)cc3)c3ccccc3-c3ccc4c(c3)c3ccccc3n4-c3ccccc3)cc21]

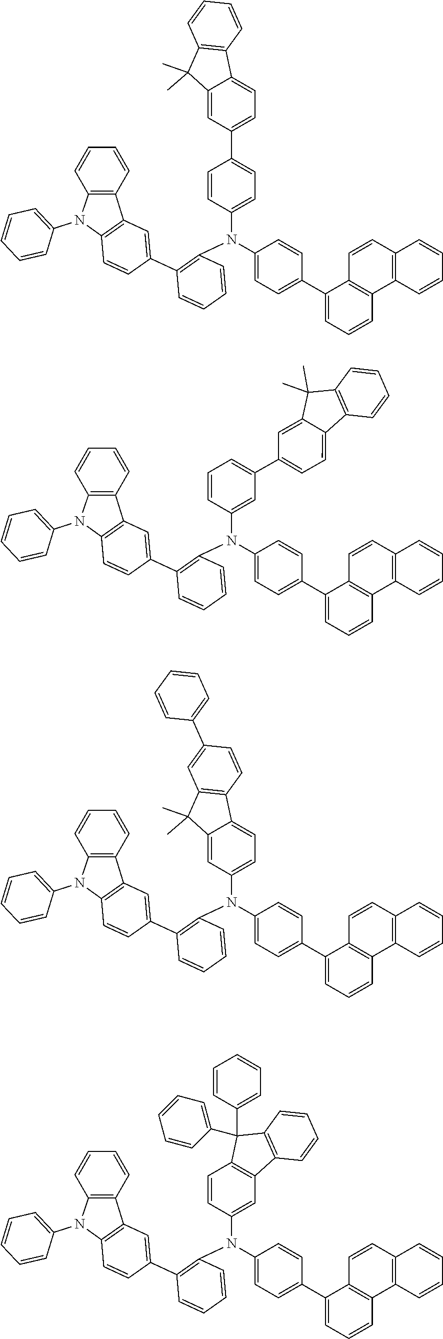

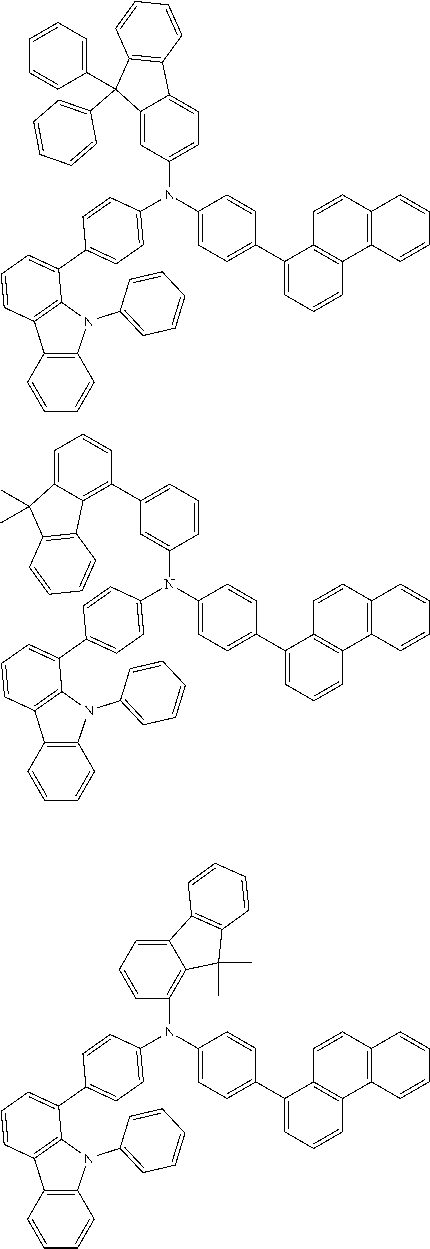

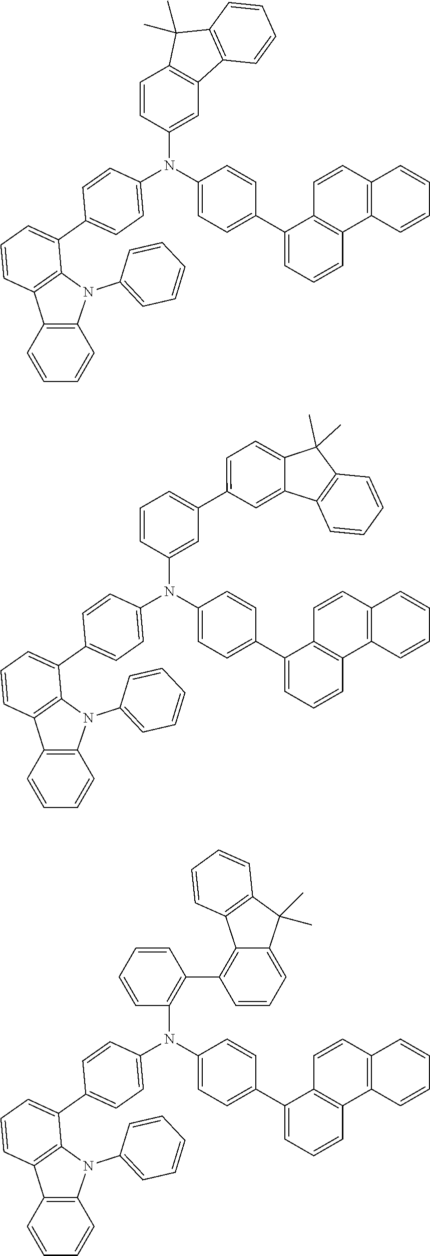 [SMILES: CC1(C)c2ccccc2-c2cc(N(c3ccc(-c4cccc5c4ccc4ccccc45)cc3)c3ccc(-c4cccc5c6ccccc6n(-c6ccccc6)c45)cc3)ccc21 ; CC1(C)c2ccccc2-c2cc(-c3cccc(N(c4ccc(-c5cccc6c5ccc5ccccc56)cc4)c4ccc(-c5cccc6c7ccccc7n(-c7ccccc7)c56)cc4)c3)ccc21 ; CC1(C)c2ccccc2-c2c(-c3ccccc3N(c3ccc(-c4cccc5c4ccc4ccccc45)cc3)c3ccc(-c4cccc5c6ccccc6n(-c6ccccc6)c45)cc3)cccc21]

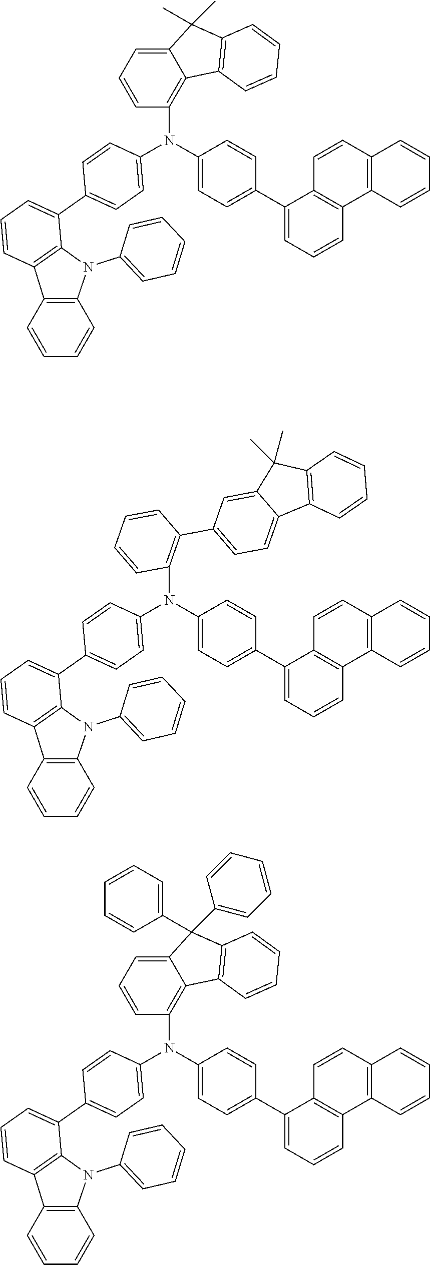 [SMILES: CC1(C)c2ccccc2-c2c(N(c3ccc(-c4cccc5c4ccc4ccccc45)cc3)c3ccc(-c4cccc5c6ccccc6n(-c6ccccc6)c45)cc3)cccc21 ; CC1(C)c2ccccc2-c2ccc(-c3ccccc3N(c3ccc(-c4cccc5c4ccc4ccccc45)cc3)c3ccc(-c4cccc5c6ccccc6n(-c6ccccc6)c45)cc3)cc21 ; c1ccc(-n2c3ccccc3c3cccc(-c4ccc(N(c5ccc(-c6cccc7c6ccc6ccccc67)cc5)c5cccc6c5-c5ccccc5C6(c5ccccc5)c5ccccc5)cc4)c32)cc1]

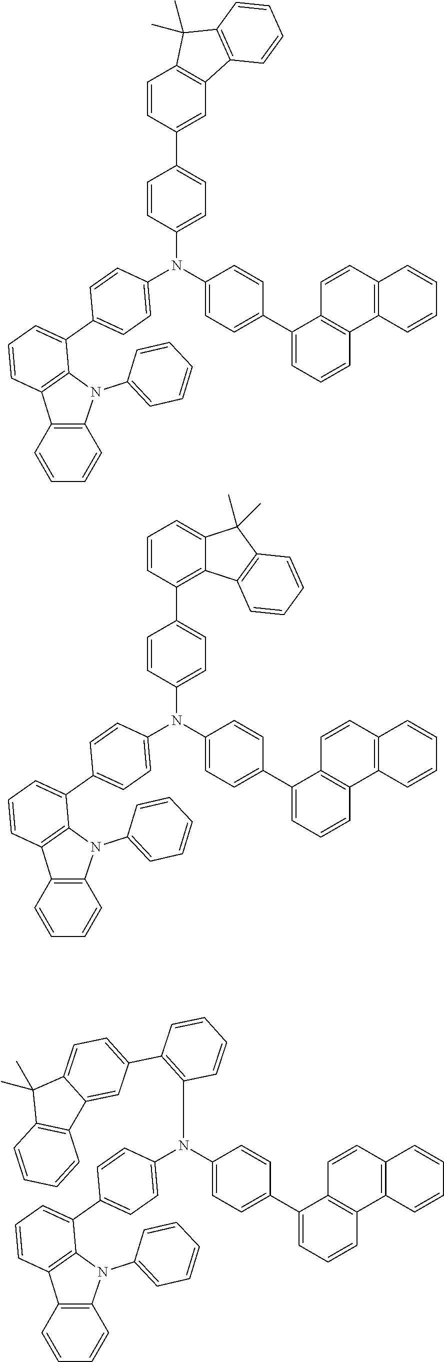 [SMILES: CC1(C)c2ccccc2-c2cc(-c3ccc(N(c4ccc(-c5cccc6c5ccc5ccccc56)cc4)c4ccc(-c5cccc6c7ccccc7n(-c7ccccc7)c56)cc4)cc3)ccc21 ; CC1(C)c2ccccc2-c2c(-c3ccc(N(c4ccc(-c5cccc6c5ccc5ccccc56)cc4)c4ccc(-c5cccc6c7ccccc7n(-c7ccccc7)c56)cc4)cc3)cccc21 ; CC1(C)c2ccccc2-c2cc(-c3ccccc3N(c3ccc(-c4cccc5c4ccc4ccccc45)cc3)c3ccc(-c4cccc5c6ccccc6n(-c6ccccc6)c45)cc3)ccc21]

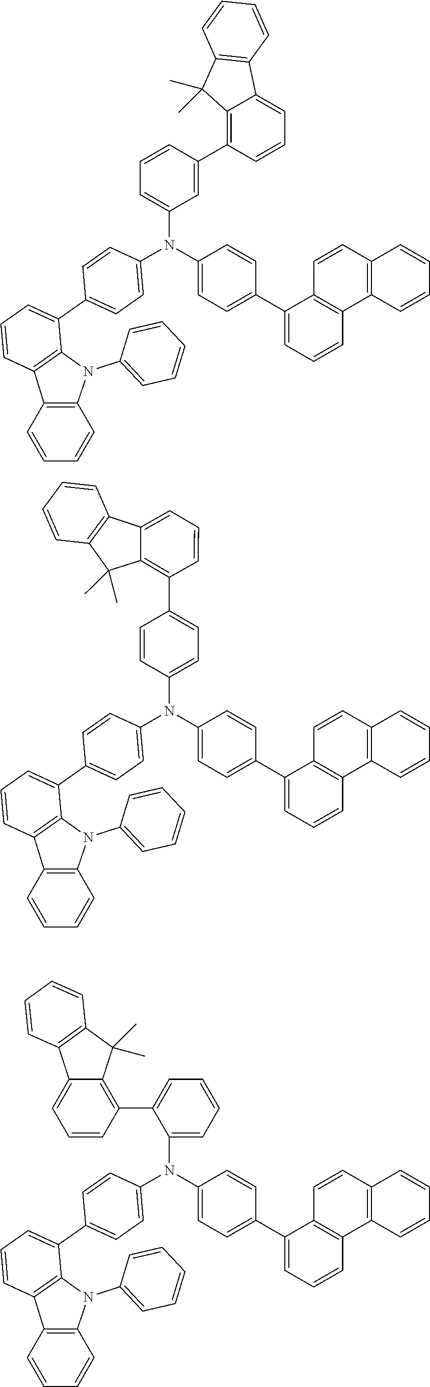

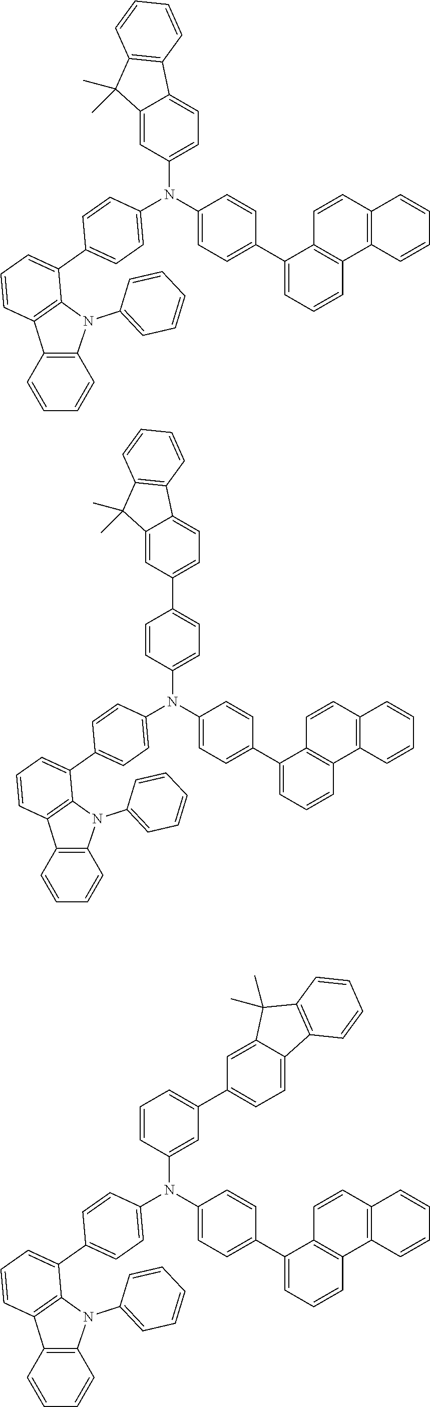 [SMILES: CC1(C)c2ccccc2-c2ccc(N(c3ccc(-c4cccc5c4ccc4ccccc45)cc3)c3ccc(-c4cccc5c6ccccc6n(-c6ccccc6)c45)cc3)cc21 ; CC1(C)c2ccccc2-c2ccc(-c3ccc(N(c4ccc(-c5cccc6c5ccc5ccccc56)cc4)c4ccc(-c5cccc6c7ccccc7n(-c7ccccc7)c56)cc4)cc3)cc21 ; CC1(C)c2ccccc2-c2ccc(-c3cccc(N(c4ccc(-c5cccc6c5ccc5ccccc56)cc4)c4ccc(-c5cccc6c7ccccc7n(-c7ccccc7)c56)cc4)c3)cc21]

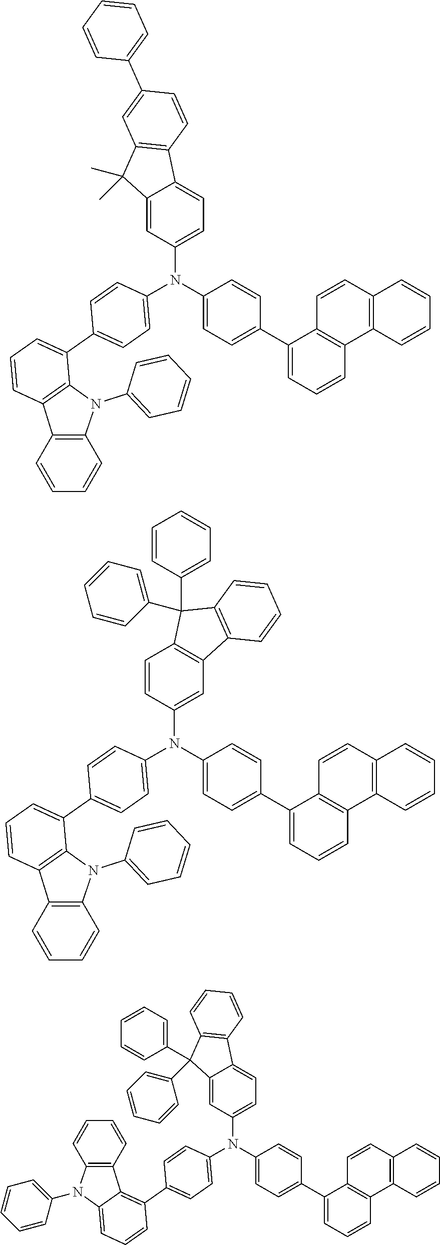 [SMILES: CC1(C)c2cc(-c3ccccc3)ccc2-c2ccc(N(c3ccc(-c4cccc5c4ccc4ccccc45)cc3)c3ccc(-c4cccc5c6ccccc6n(-c6ccccc6)c45)cc3)cc21 ; c1ccc(-n2c3ccccc3c3cccc(-c4ccc(N(c5ccc(-c6cccc7c6ccc6ccccc67)cc5)c5ccc6c(c5)-c5ccccc5C6(c5ccccc5)c5ccccc5)cc4)c32)cc1 ; c1ccc(-n2c3ccccc3c3c(-c4ccc(N(c5ccc(-c6cccc7c6ccc6ccccc67)cc5)c5ccc6c(c5)C(c5ccccc5)(c5ccccc5)c5ccccc5-6)cc4)cccc32)cc1]

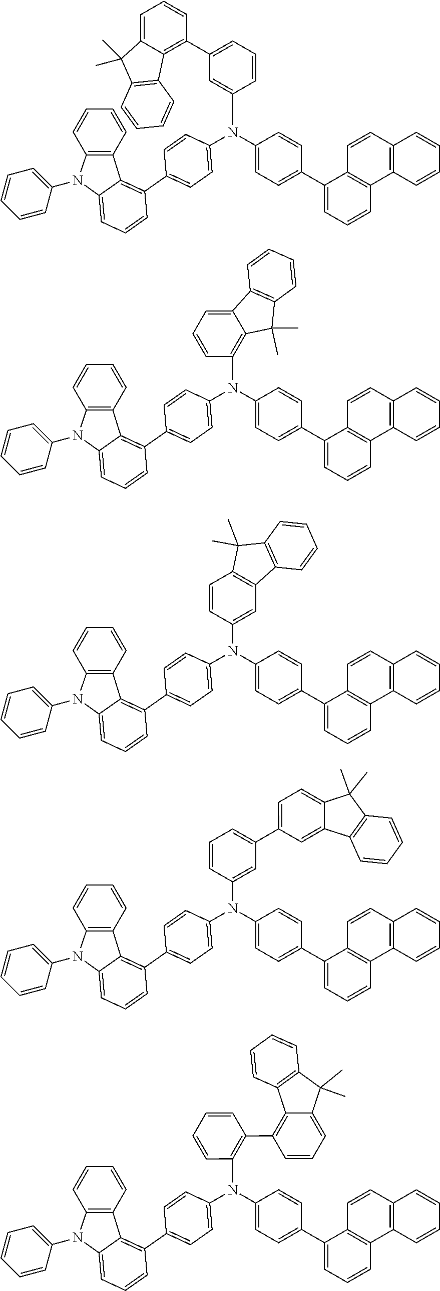

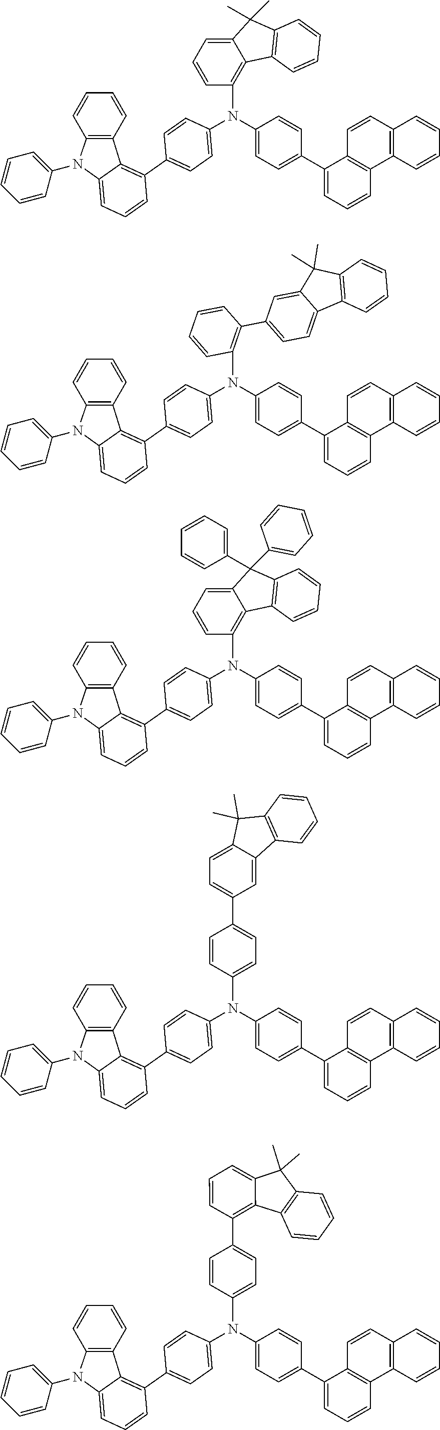

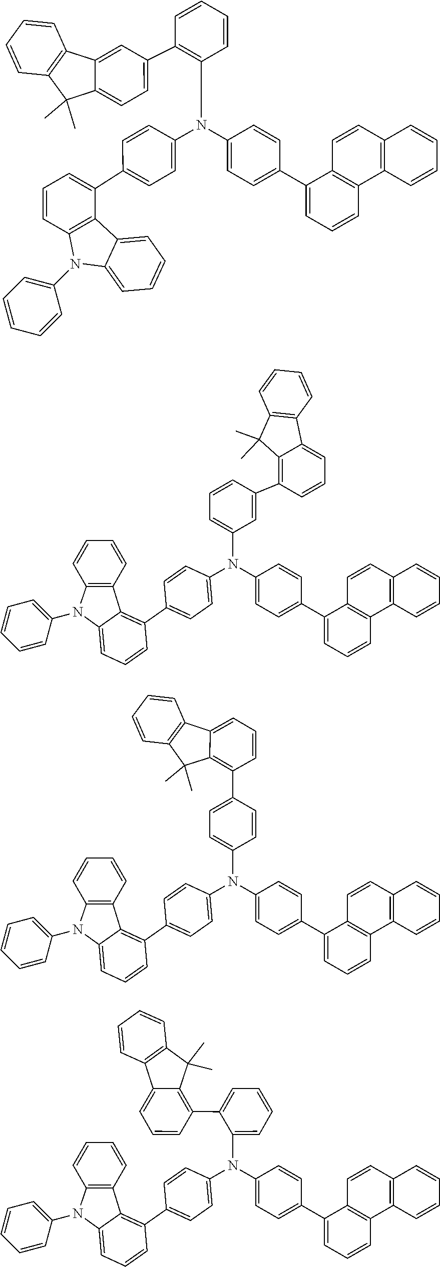

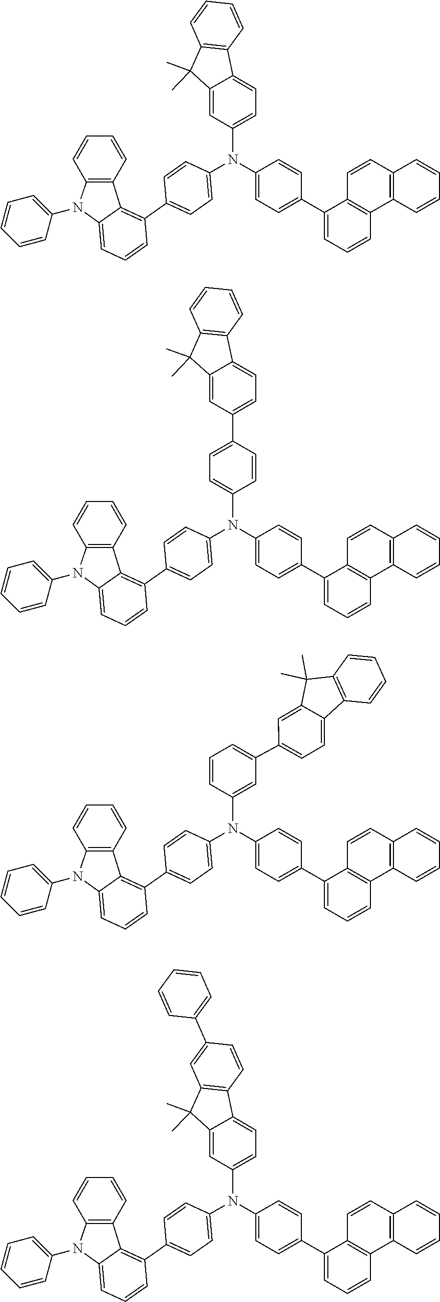 [SMILES: CC1(C)c2ccccc2-c2ccc(N(c3ccc(-c4cccc5c4ccc4ccccc45)cc3)c3ccc(-c4cccc5c4c4ccccc4n5-c4ccccc4)cc3)cc21 ; CC1(C)c2ccccc2-c2ccc(-c3ccc(N(c4ccc(-c5cccc6c5ccc5ccccc56)cc4)c4ccc(-c5cccc6c5c5ccccc5n6-c5ccccc5)cc4)cc3)cc21 ; CC1(C)c2ccccc2-c2ccc(-c3cccc(N(c4ccc(-c5cccc6c5ccc5ccccc56)cc4)c4ccc(-c5cccc6c5c5ccccc5n6-c5ccccc5)cc4)c3)cc21 ; CC1(C)c2cc(-c3ccccc3)ccc2-c2ccc(N(c3ccc(-c4cccc5c4ccc4ccccc45)cc3)c3ccc(-c4cccc5c4c4ccccc4n5-c4ccccc4)cc3)cc21]

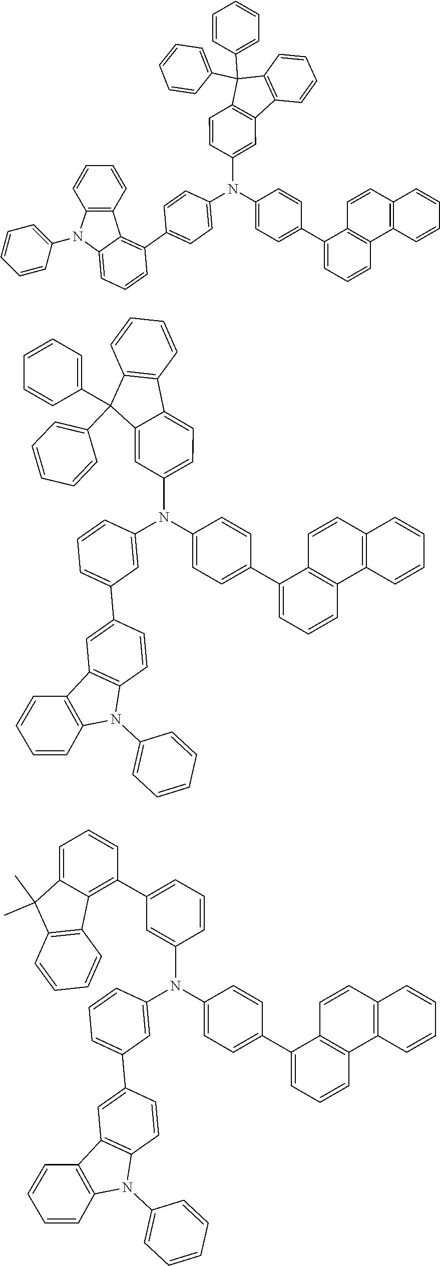

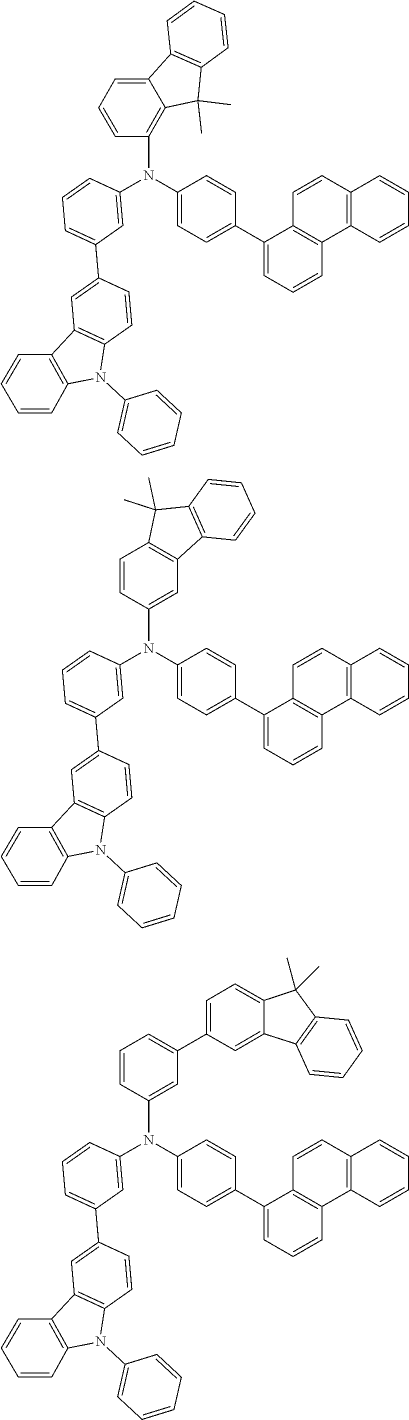

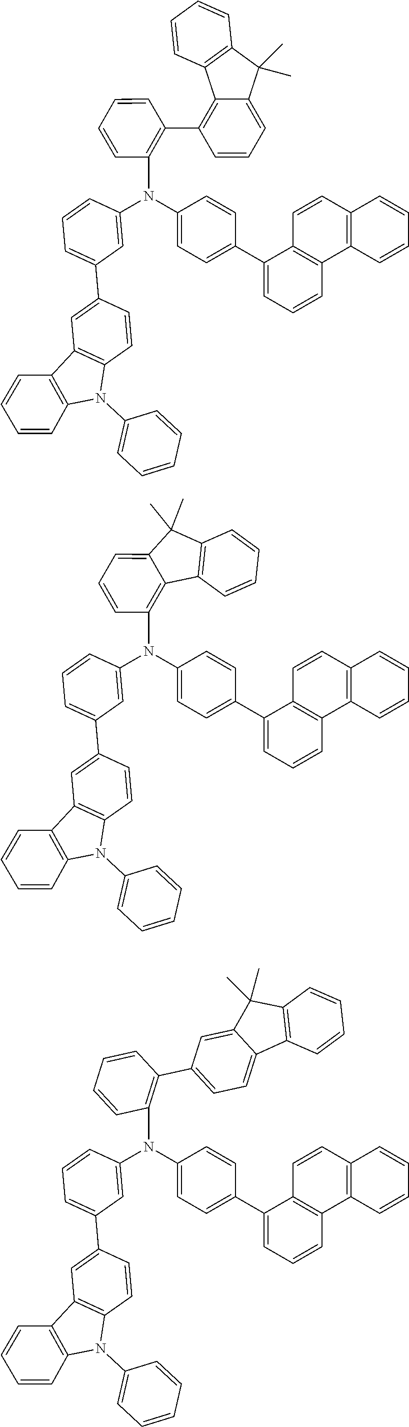

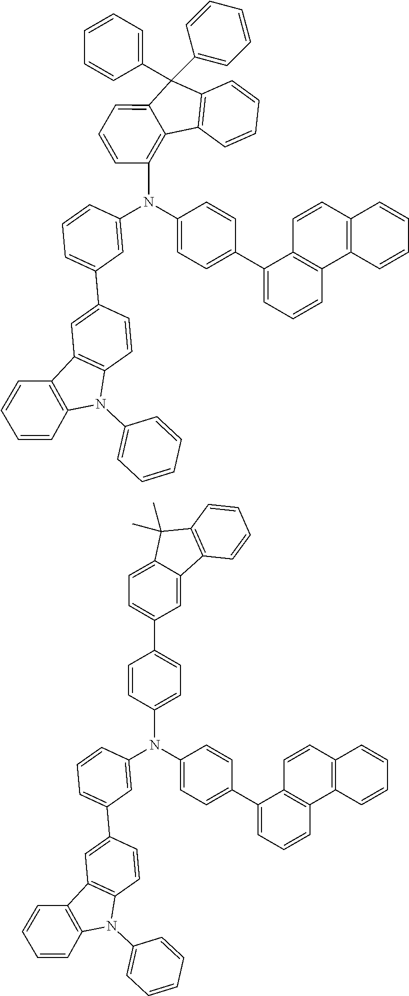

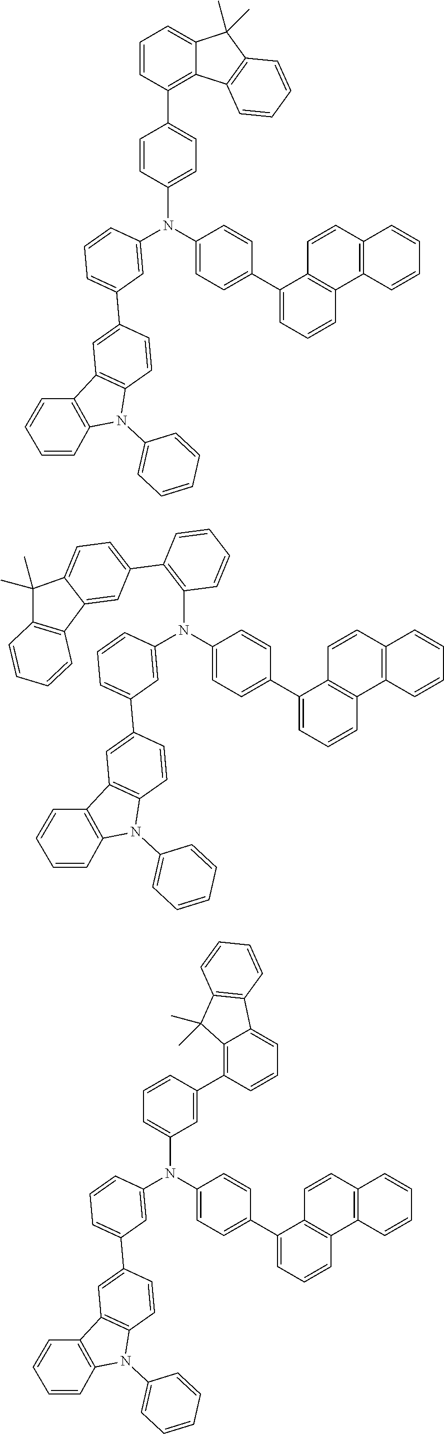

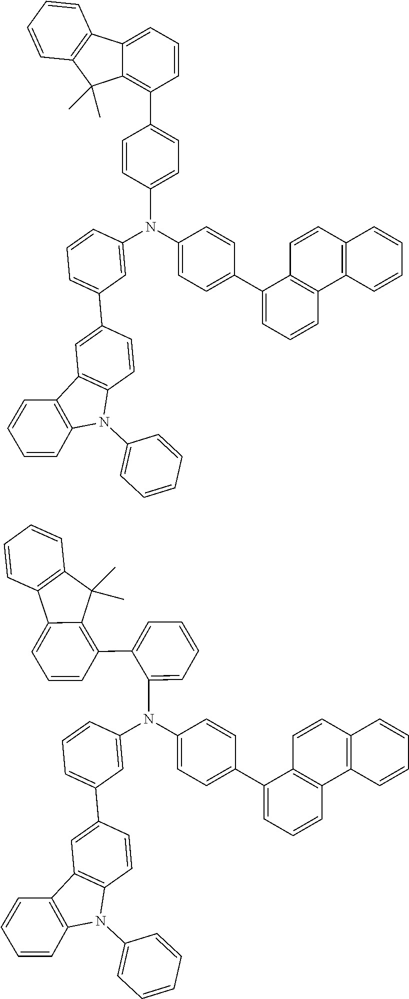 [SMILES: CC1(C)c2ccccc2-c2cccc(-c3ccc(N(c4ccc(-c5cccc6c5ccc5ccccc56)cc4)c4cccc(-c5ccc6c(c5)c5ccccc5n6-c5ccccc5)c4)cc3)c21 ; CC1(C)c2ccccc2-c2cccc(-c3ccccc3N(c3ccc(-c4cccc5c4ccc4ccccc45)cc3)c3cccc(-c4ccc5c(c4)c4ccccc4n5-c4ccccc4)c3)c21]

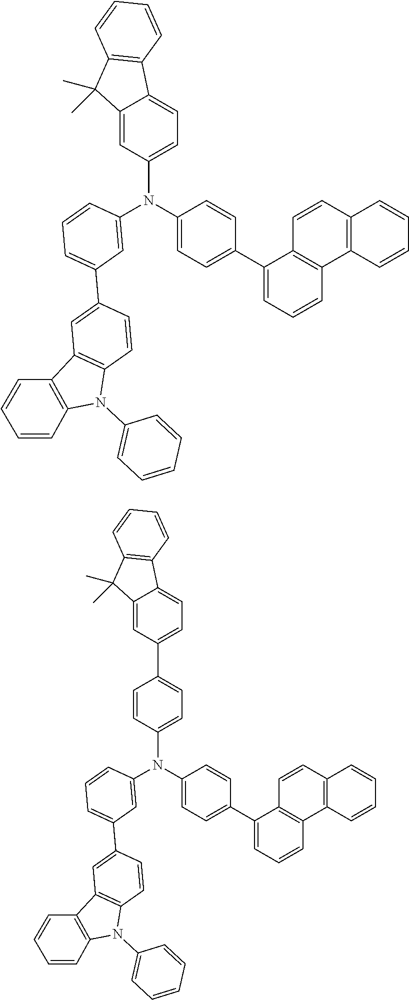

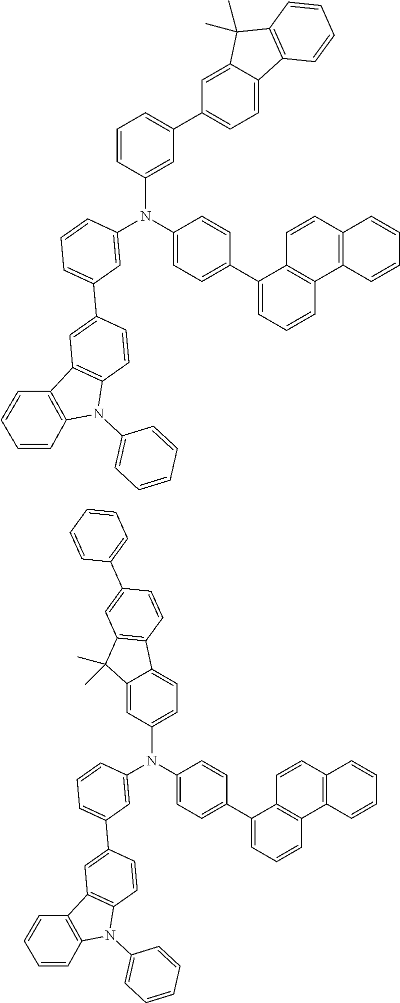

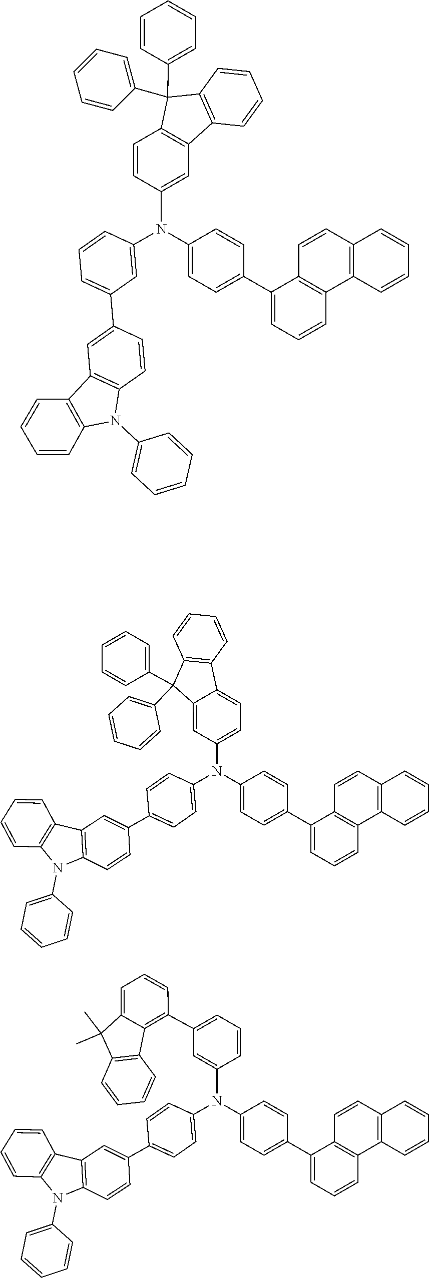 [SMILES: c1ccc(-n2c3ccccc3c3cc(-c4cccc(N(c5ccc(-c6cccc7c6ccc6ccccc67)cc5)c5ccc6c(c5)-c5ccccc5C6(c5ccccc5)c5ccccc5)c4)ccc32)cc1 ; c1ccc(-n2c3ccccc3c3cc(-c4ccc(N(c5ccc(-c6cccc7c6ccc6ccccc67)cc5)c5ccc6c(c5)C(c5ccccc5)(c5ccccc5)c5ccccc5-6)cc4)ccc32)cc1 ; CC1(C)c2ccccc2-c2c(-c3cccc(N(c4ccc(-c5ccc6c(c5)c5ccccc5n6-c5ccccc5)cc4)c4ccc(-c5cccc6c5ccc5ccccc56)cc4)c3)cccc21]

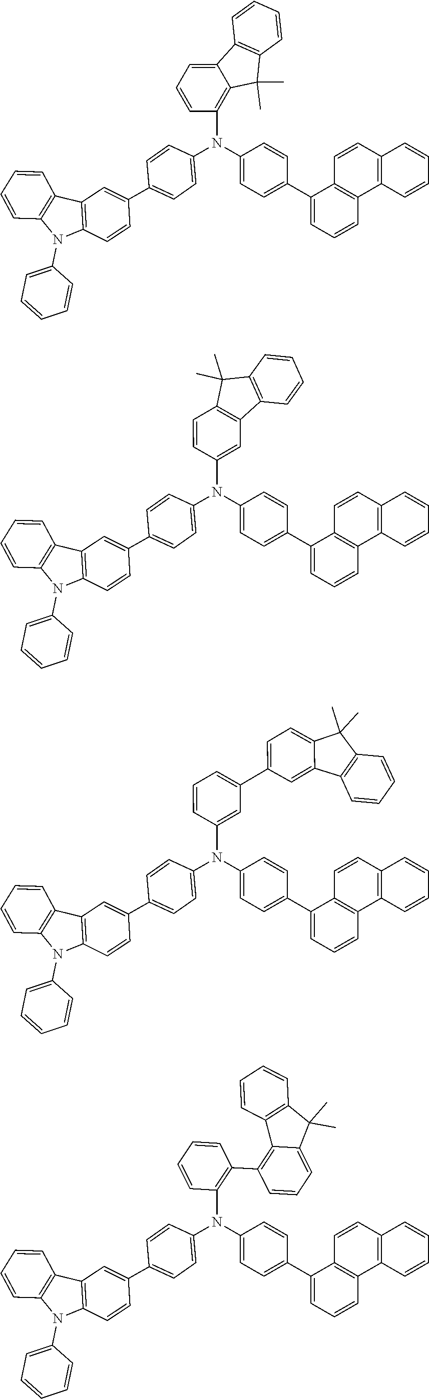 [SMILES: CC1(C)c2ccccc2-c2cccc(N(c3ccc(-c4ccc5c(c4)c4ccccc4n5-c4ccccc4)cc3)c3ccc(-c4cccc5c4ccc4ccccc45)cc3)c21 ; CC1(C)c2ccccc2-c2cc(N(c3ccc(-c4ccc5c(c4)c4ccccc4n5-c4ccccc4)cc3)c3ccc(-c4cccc5c4ccc4ccccc45)cc3)ccc21 ; CC1(C)c2ccccc2-c2cc(-c3cccc(N(c4ccc(-c5ccc6c(c5)c5ccccc5n6-c5ccccc5)cc4)c4ccc(-c5cccc6c5ccc5ccccc56)cc4)c3)ccc21 ; CC1(C)c2ccccc2-c2c(-c3ccccc3N(c3ccc(-c4ccc5c(c4)c4ccccc4n5-c4ccccc4)cc3)c3ccc(-c4cccc5c4ccc4ccccc45)cc3)cccc21]

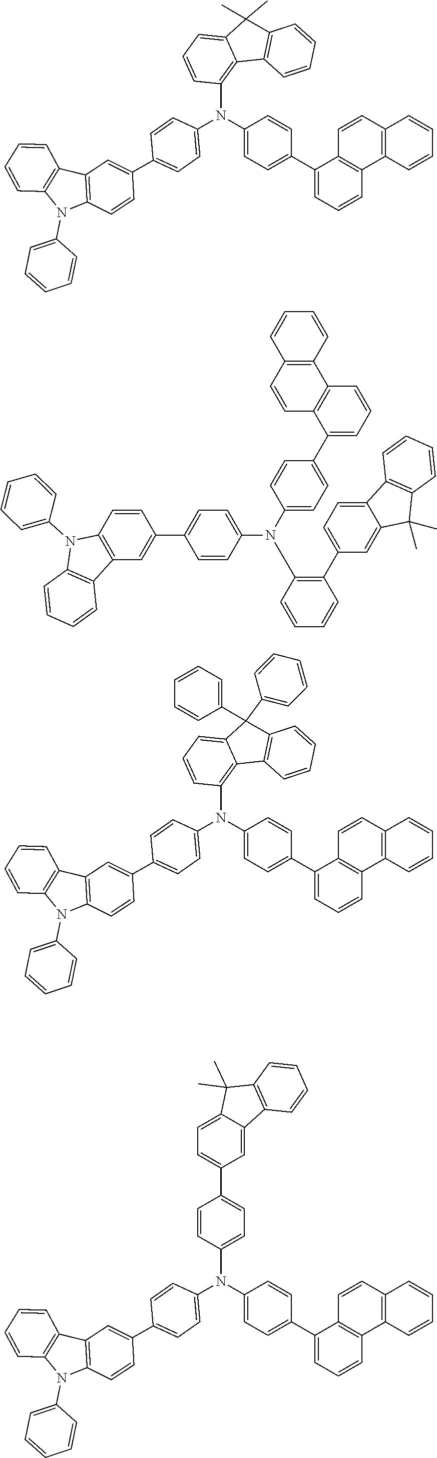

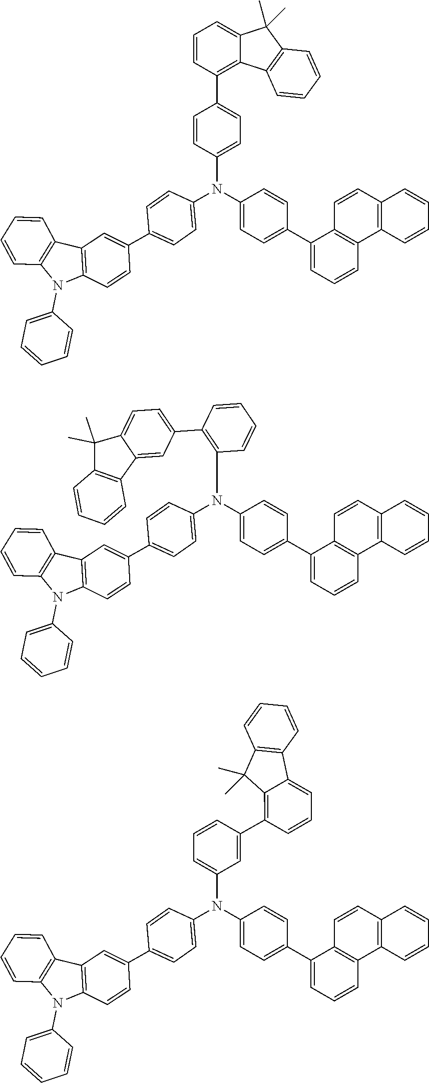 [SMILES: CC1(C)c2ccccc2-c2c(-c3ccc(N(c4ccc(-c5ccc6c(c5)c5ccccc5n6-c5ccccc5)cc4)c4ccc(-c5cccc6c5ccc5ccccc56)cc4)cc3)cccc21 ; CC1(C)c2ccccc2-c2cc(-c3ccccc3N(c3ccc(-c4ccc5c(c4)c4ccccc4n5-c4ccccc4)cc3)c3ccc(-c4cccc5c4ccc4ccccc45)cc3)ccc21 ; CC1(C)c2ccccc2-c2cccc(-c3cccc(N(c4ccc(-c5ccc6c(c5)c5ccccc5n6-c5ccccc5)cc4)c4ccc(-c5cccc6c5ccc5ccccc56)cc4)c3)c21]

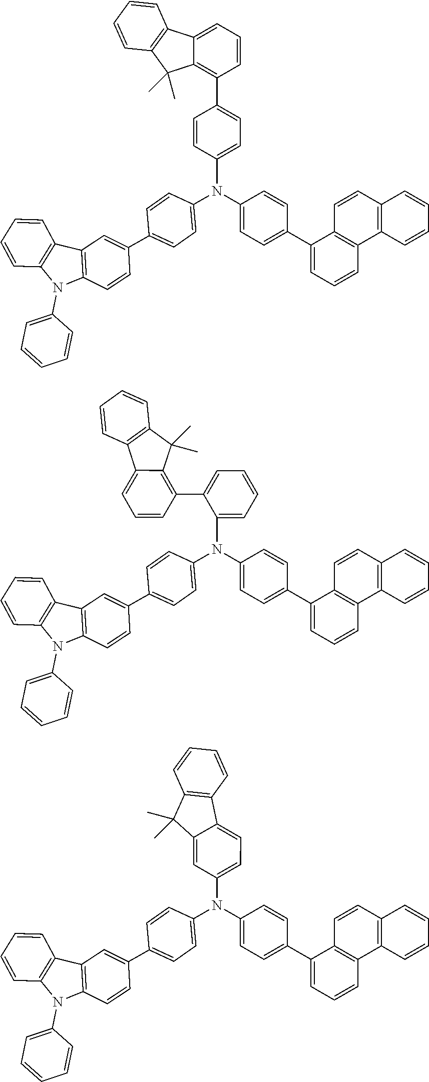 [SMILES: CC1(C)c2ccccc2-c2cccc(-c3ccc(N(c4ccc(-c5ccc6c(c5)c5ccccc5n6-c5ccccc5)cc4)c4ccc(-c5cccc6c5ccc5ccccc56)cc4)cc3)c21 ; CC1(C)c2ccccc2-c2cccc(-c3ccccc3N(c3ccc(-c4ccc5c(c4)c4ccccc4n5-c4ccccc4)cc3)c3ccc(-c4cccc5c4ccc4ccccc45)cc3)c21 ; CC1(C)c2ccccc2-c2ccc(N(c3ccc(-c4ccc5c(c4)c4ccccc4n5-c4ccccc4)cc3)c3ccc(-c4cccc5c4ccc4ccccc45)cc3)cc21]

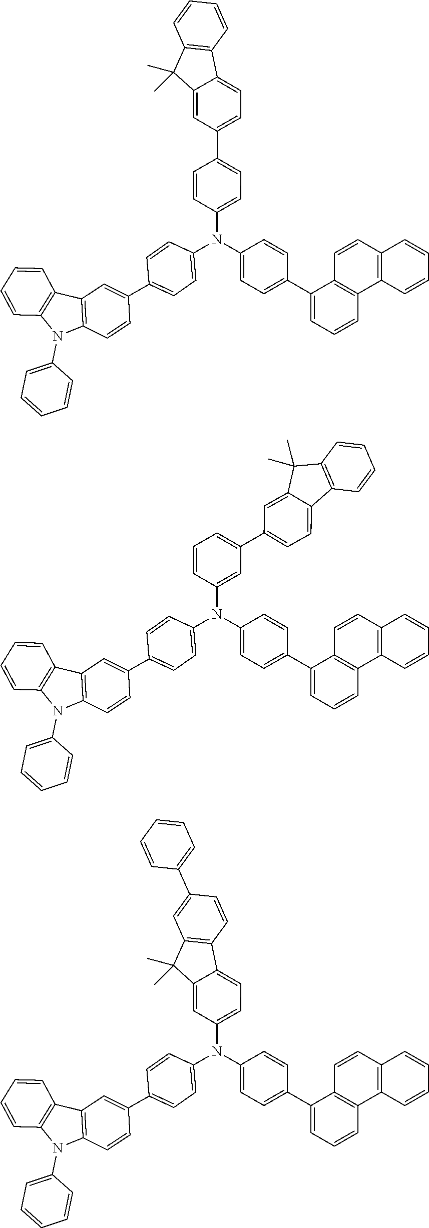 [SMILES: CC1(C)c2ccccc2-c2ccc(-c3ccc(N(c4ccc(-c5ccc6c(c5)c5ccccc5n6-c5ccccc5)cc4)c4ccc(-c5cccc6c5ccc5ccccc56)cc4)cc3)cc21 ; CC1(C)c2ccccc2-c2ccc(-c3cccc(N(c4ccc(-c5ccc6c(c5)c5ccccc5n6-c5ccccc5)cc4)c4ccc(-c5cccc6c5ccc5ccccc56)cc4)c3)cc21 ; CC1(C)c2cc(-c3ccccc3)ccc2-c2ccc(N(c3ccc(-c4ccc5c(c4)c4ccccc4n5-c4ccccc4)cc3)c3ccc(-c4cccc5c4ccc4ccccc45)cc3)cc21]

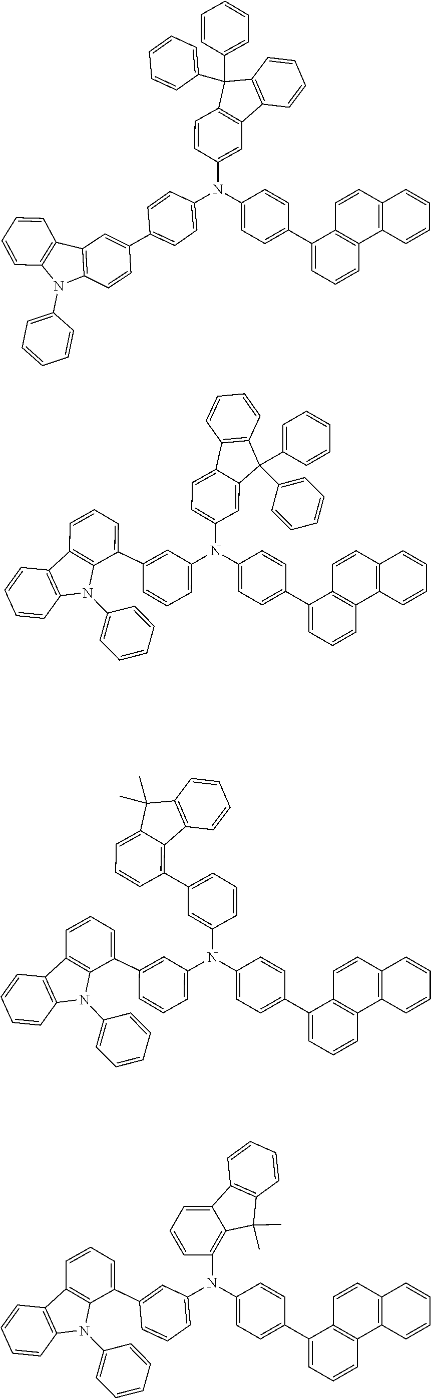 [SMILES: c1ccc(-n2c3ccccc3c3cc(-c4ccc(N(c5ccc(-c6cccc7c6ccc6ccccc67)cc5)c5ccc6c(c5)-c5ccccc5C6(c5ccccc5)c5ccccc5)cc4)ccc32)cc1 ; c1ccc(-n2c3ccccc3c3cccc(-c4cccc(N(c5ccc(-c6cccc7c6ccc6ccccc67)cc5)c5ccc6c(c5)C(c5ccccc5)(c5ccccc5)c5ccccc5-6)c4)c32)cc1 ; CC1(C)c2ccccc2-c2c(-c3cccc(N(c4ccc(-c5cccc6c5ccc5ccccc56)cc4)c4cccc(-c5cccc6c7ccccc7n(-c7ccccc7)c56)c4)c3)cccc21 ; CC1(C)c2ccccc2-c2cccc(N(c3ccc(-c4cccc5c4ccc4ccccc45)cc3)c3cccc(-c4cccc5c6ccccc6n(-c6ccccc6)c45)c3)c21]

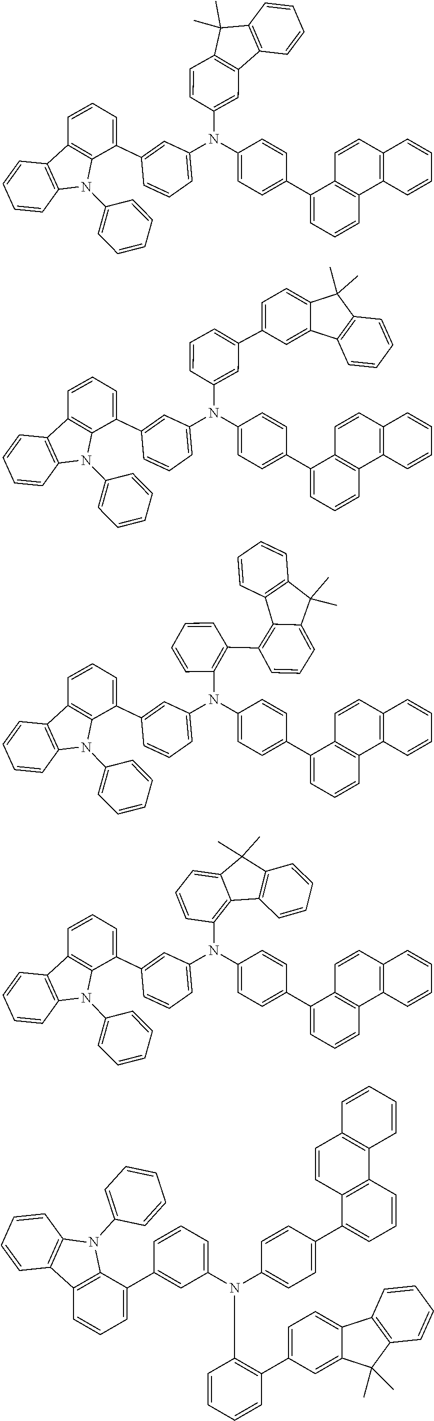

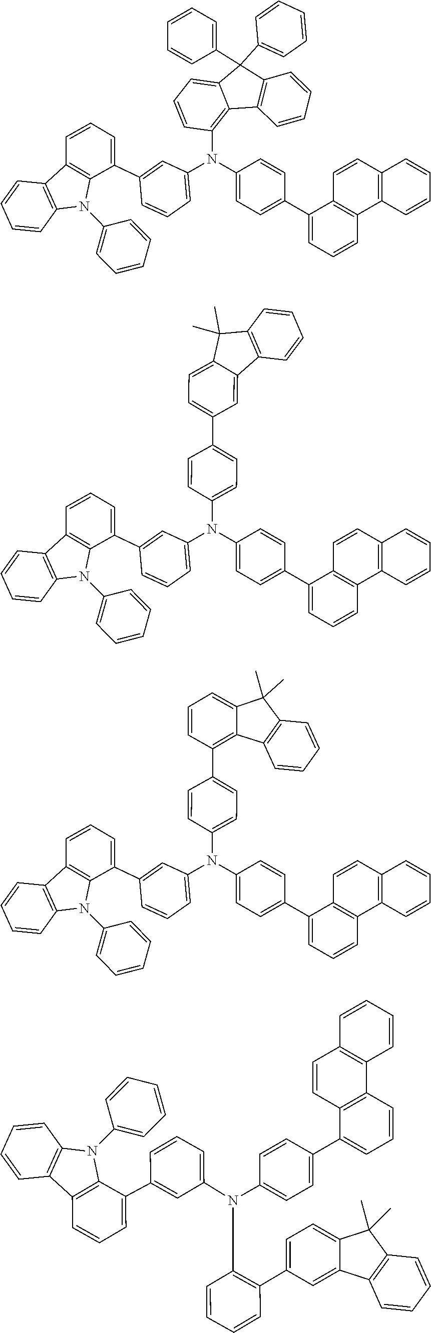 [SMILES: c1ccc(-n2c3ccccc3c3cccc(-c4cccc(N(c5ccc(-c6cccc7c6ccc6ccccc67)cc5)c5cccc6c5-c5ccccc5C6(c5ccccc5)c5ccccc5)c4)c32)cc1 ; CC1(C)c2ccccc2-c2cc(-c3ccc(N(c4ccc(-c5cccc6c5ccc5ccccc56)cc4)c4cccc(-c5cccc6c7ccccc7n(-c7ccccc7)c56)c4)cc3)ccc21 ; CC1(C)c2ccccc2-c2c(-c3ccc(N(c4ccc(-c5cccc6c5ccc5ccccc56)cc4)c4cccc(-c5cccc6c7ccccc7n(-c7ccccc7)c56)c4)cc3)cccc21 ; CC1(C)c2ccccc2-c2cc(-c3ccccc3N(c3ccc(-c4cccc5c4ccc4ccccc45)cc3)c3cccc(-c4cccc5c6ccccc6n(-c6ccccc6)c45)c3)ccc21]

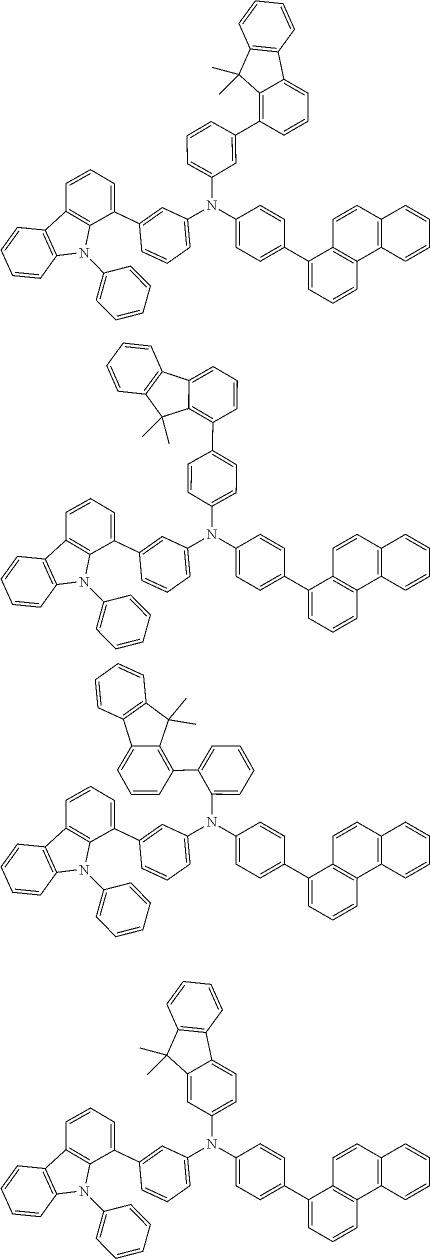

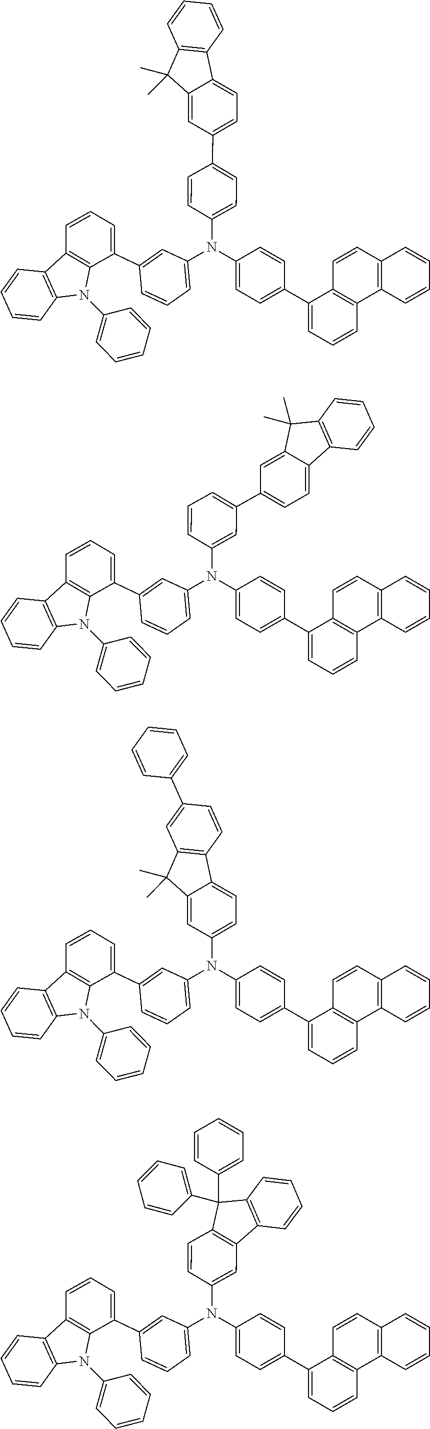 [SMILES: CC1(C)c2ccccc2-c2ccc(-c3ccc(N(c4ccc(-c5cccc6c5ccc5ccccc56)cc4)c4cccc(-c5cccc6c7ccccc7n(-c7ccccc7)c56)c4)cc3)cc21 ; CC1(C)c2ccccc2-c2ccc(-c3cccc(N(c4ccc(-c5cccc6c5ccc5ccccc56)cc4)c4cccc(-c5cccc6c7ccccc7n(-c7ccccc7)c56)c4)c3)cc21 ; CC1(C)c2cc(-c3ccccc3)ccc2-c2ccc(N(c3ccc(-c4cccc5c4ccc4ccccc45)cc3)c3cccc(-c4cccc5c6ccccc6n(-c6ccccc6)c45)c3)cc21 ; c1ccc(-n2c3ccccc3c3cccc(-c4cccc(N(c5ccc(-c6cccc7c6ccc6ccccc67)cc5)c5ccc6c(c5)-c5ccccc5C6(c5ccccc5)c5ccccc5)c4)c32)cc1]

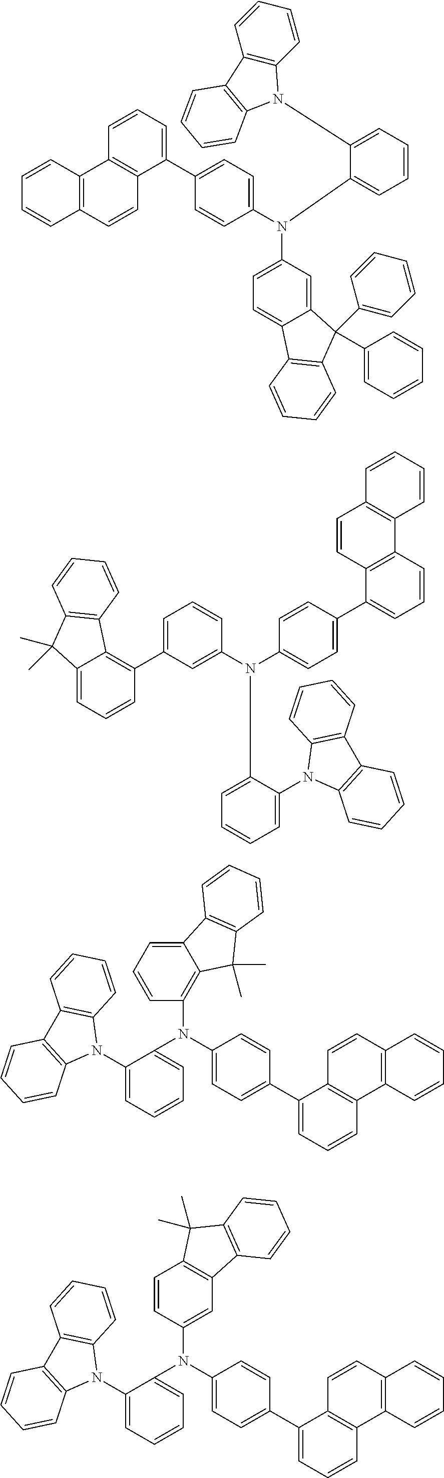

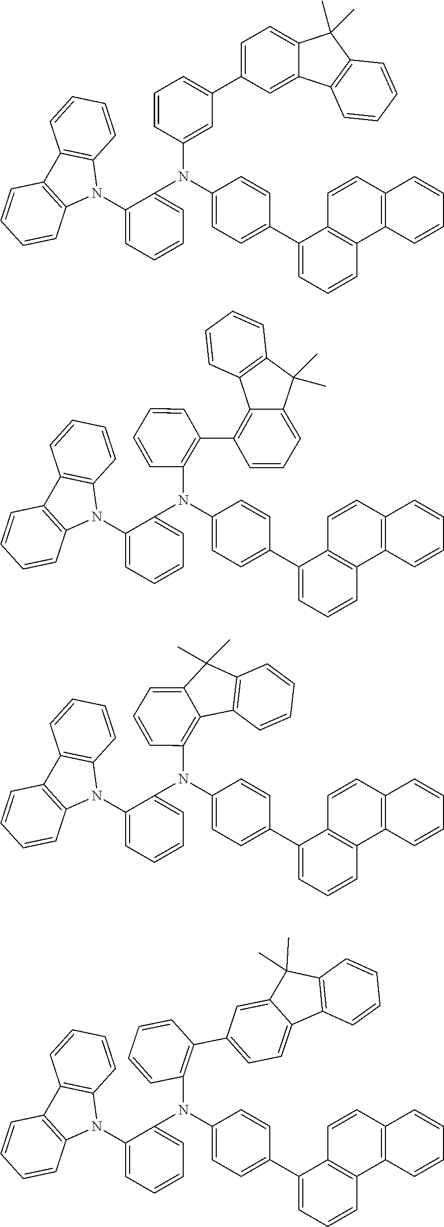 [SMILES: CC1(C)c2ccccc2-c2cc(-c3cccc(N(c4ccc(-c5cccc6c5ccc5ccccc56)cc4)c4ccccc4-n4c5ccccc5c5ccccc54)c3)ccc21 ; CC1(C)c2ccccc2-c2c(-c3ccccc3N(c3ccc(-c4cccc5c4ccc4ccccc45)cc3)c3ccccc3-n3c4ccccc4c4ccccc43)cccc21 ; CC1(C)c2ccccc2-c2c(N(c3ccc(-c4cccc5c4ccc4ccccc45)cc3)c3ccccc3-n3c4ccccc4c4ccccc43)cccc21 ; CC1(C)c2ccccc2-c2ccc(-c3ccccc3N(c3ccc(-c4cccc5c4ccc4ccccc45)cc3)c3ccccc3-n3c4ccccc4c4ccccc43)cc21]

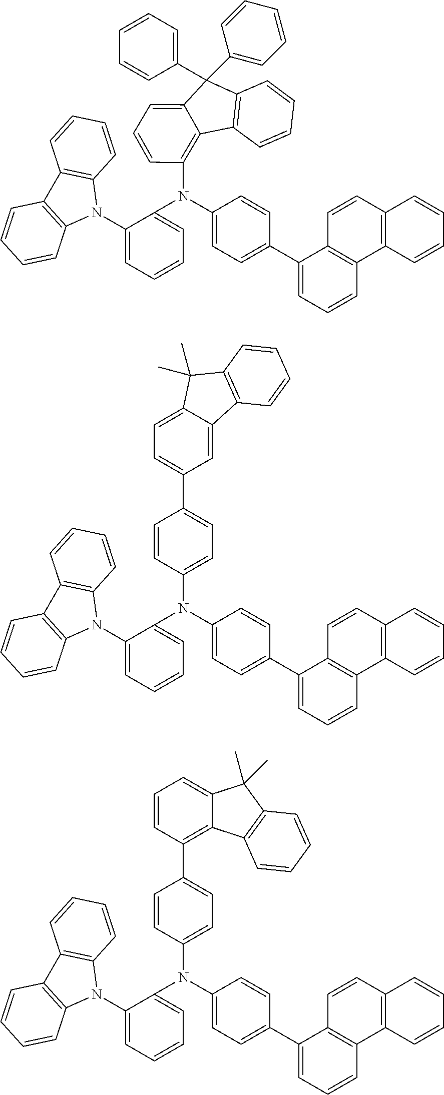 [SMILES: c1ccc(C2(c3ccccc3)c3ccccc3-c3c(N(c4ccc(-c5cccc6c5ccc5ccccc56)cc4)c4ccccc4-n4c5ccccc5c5ccccc54)cccc32)cc1 ; CC1(C)c2ccccc2-c2cc(-c3ccc(N(c4ccc(-c5cccc6c5ccc5ccccc56)cc4)c4ccccc4-n4c5ccccc5c5ccccc54)cc3)ccc21 ; CC1(C)c2ccccc2-c2c(-c3ccc(N(c4ccc(-c5cccc6c5ccc5ccccc56)cc4)c4ccccc4-n4c5ccccc5c5ccccc54)cc3)cccc21]

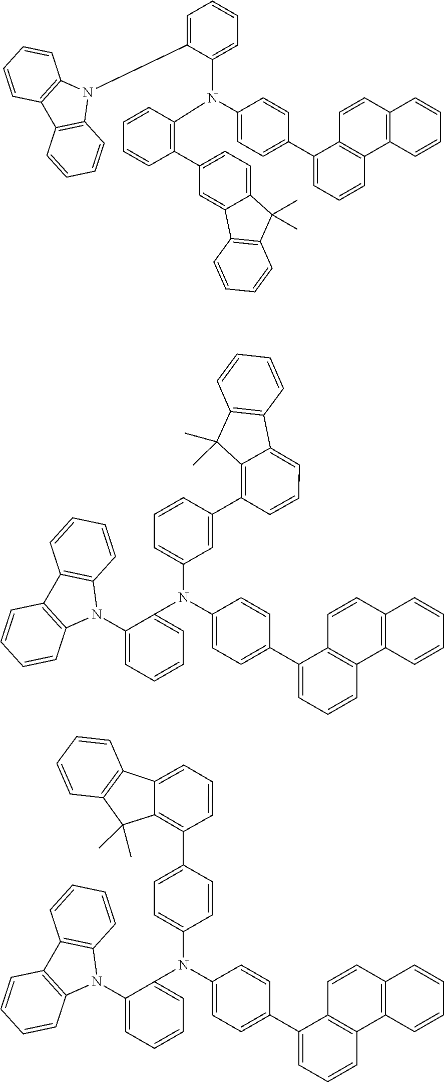 [SMILES: CC1(C)c2ccccc2-c2cc(-c3ccccc3N(c3ccc(-c4cccc5c4ccc4ccccc45)cc3)c3ccccc3-n3c4ccccc4c4ccccc43)ccc21 ; CC1(C)c2ccccc2-c2cccc(-c3cccc(N(c4ccc(-c5cccc6c5ccc5ccccc56)cc4)c4ccccc4-n4c5ccccc5c5ccccc54)c3)c21 ; CC1(C)c2ccccc2-c2cccc(-c3ccc(N(c4ccc(-c5cccc6c5ccc5ccccc56)cc4)c4ccccc4-n4c5ccccc5c5ccccc54)cc3)c21]

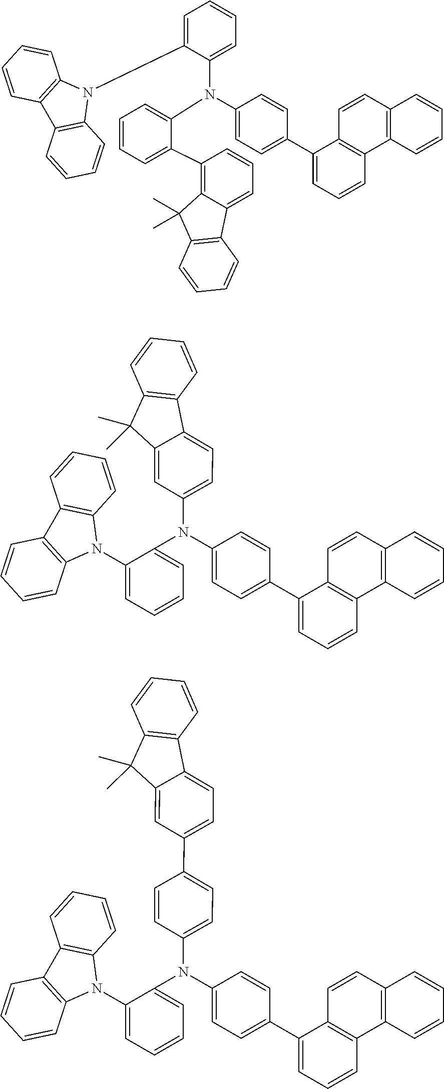 [SMILES: CC1(C)c2ccccc2-c2cccc(-c3ccccc3N(c3ccc(-c4cccc5c4ccc4ccccc45)cc3)c3ccccc3-n3c4ccccc4c4ccccc43)c21 ; CC1(C)c2ccccc2-c2ccc(N(c3ccc(-c4cccc5c4ccc4ccccc45)cc3)c3ccccc3-n3c4ccccc4c4ccccc43)cc21 ; CC1(C)c2ccccc2-c2ccc(-c3ccc(N(c4ccc(-c5cccc6c5ccc5ccccc56)cc4)c4ccccc4-n4c5ccccc5c5ccccc54)cc3)cc21]

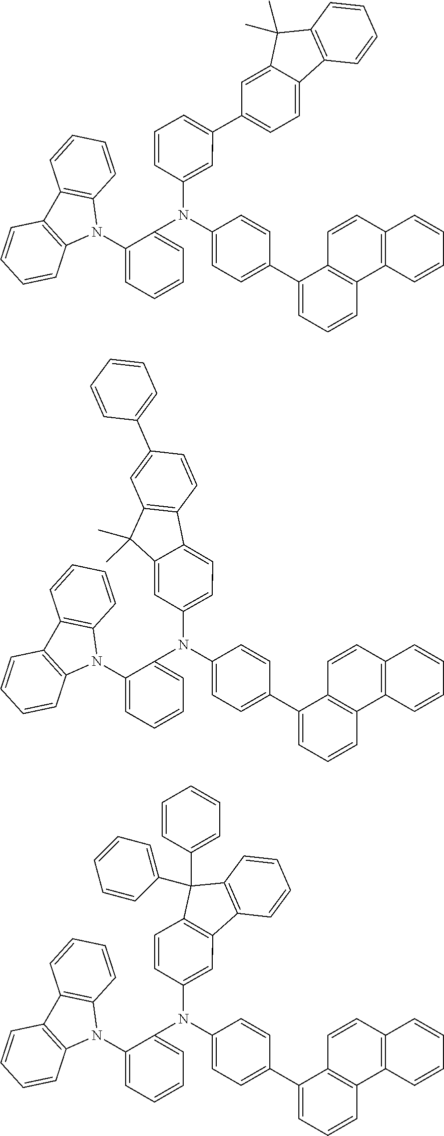 [SMILES: CC1(C)c2ccccc2-c2ccc(-c3cccc(N(c4ccc(-c5cccc6c5ccc5ccccc56)cc4)c4ccccc4-n4c5ccccc5c5ccccc54)c3)cc21 ; CC1(C)c2cc(-c3ccccc3)ccc2-c2ccc(N(c3ccc(-c4cccc5c4ccc4ccccc45)cc3)c3ccccc3-n3c4ccccc4c4ccccc43)cc21 ; c1ccc(C2(c3ccccc3)c3ccccc3-c3cc(N(c4ccc(-c5cccc6c5ccc5ccccc56)cc4)c4ccccc4-n4c5ccccc5c5ccccc54)ccc32)cc1]

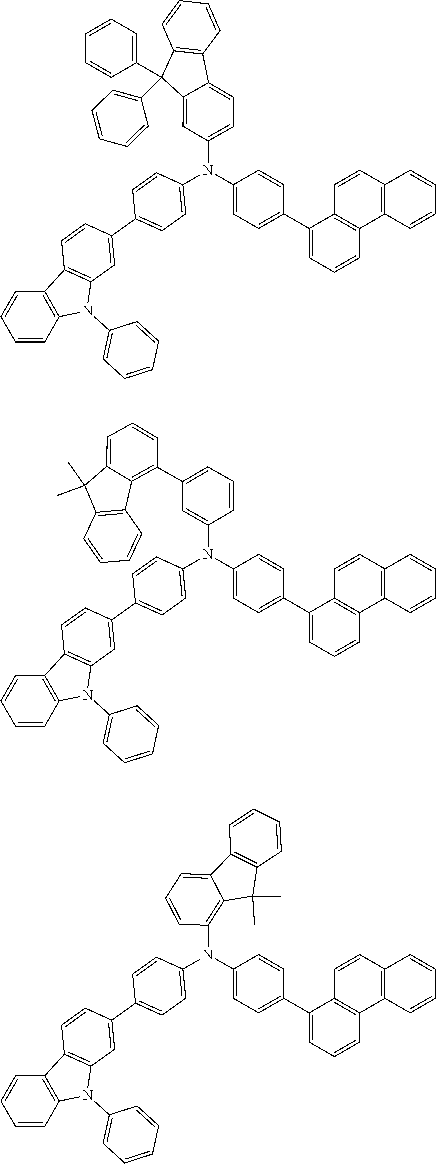 [SMILES: c1ccc(-n2c3ccccc3c3ccc(-c4ccc(N(c5ccc(-c6cccc7c6ccc6ccccc67)cc5)c5ccc6c(c5)C(c5ccccc5)(c5ccccc5)c5ccccc5-6)cc4)cc32)cc1 ; CC1(C)c2ccccc2-c2c(-c3cccc(N(c4ccc(-c5ccc6c7ccccc7n(-c7ccccc7)c6c5)cc4)c4ccc(-c5cccc6c5ccc5ccccc56)cc4)c3)cccc21 ; CC1(C)c2ccccc2-c2cccc(N(c3ccc(-c4ccc5c6ccccc6n(-c6ccccc6)c5c4)cc3)c3ccc(-c4cccc5c4ccc4ccccc45)cc3)c21]

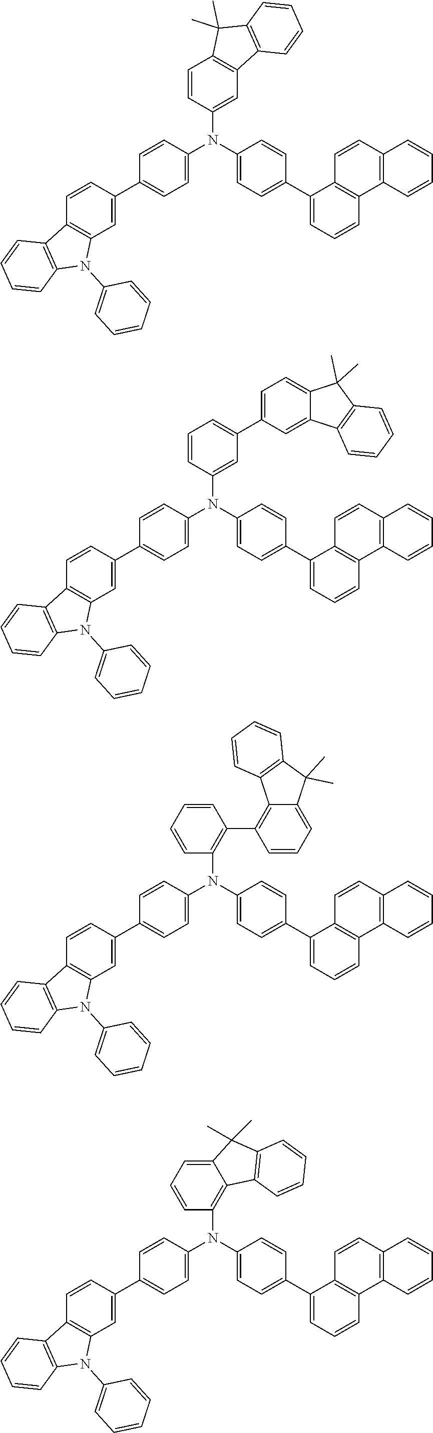 [SMILES: CC1(C)c2ccccc2-c2cc(N(c3ccc(-c4ccc5c6ccccc6n(-c6ccccc6)c5c4)cc3)c3ccc(-c4cccc5c4ccc4ccccc45)cc3)ccc21 ; CC1(C)c2ccccc2-c2cc(-c3cccc(N(c4ccc(-c5ccc6c7ccccc7n(-c7ccccc7)c6c5)cc4)c4ccc(-c5cccc6c5ccc5ccccc56)cc4)c3)ccc21 ; CC1(C)c2ccccc2-c2c(-c3ccccc3N(c3ccc(-c4ccc5c6ccccc6n(-c6ccccc6)c5c4)cc3)c3ccc(-c4cccc5c4ccc4ccccc45)cc3)cccc21 ; CC1(C)c2ccccc2-c2c(N(c3ccc(-c4ccc5c6ccccc6n(-c6ccccc6)c5c4)cc3)c3ccc(-c4cccc5c4ccc4ccccc45)cc3)cccc21]

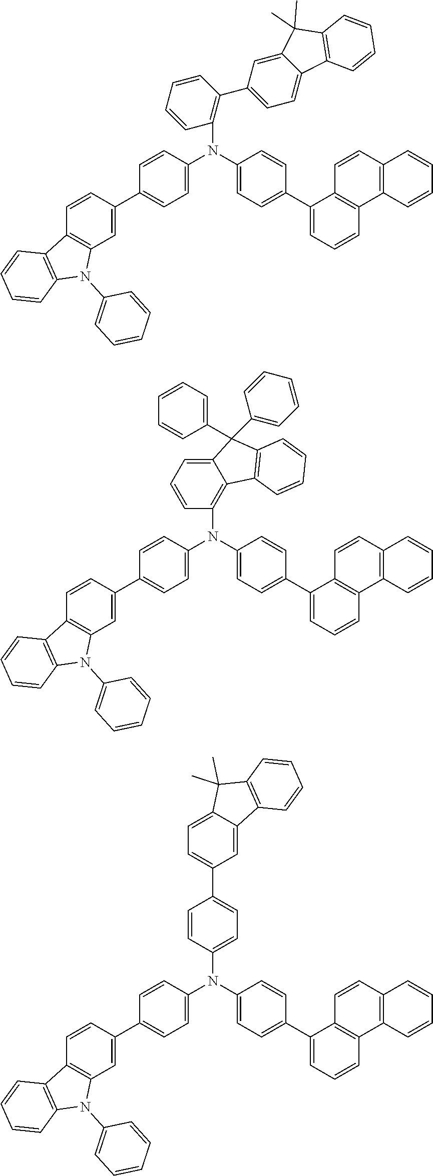 [SMILES: CC1(C)c2ccccc2-c2ccc(-c3ccccc3N(c3ccc(-c4ccc5c6ccccc6n(-c6ccccc6)c5c4)cc3)c3ccc(-c4cccc5c4ccc4ccccc45)cc3)cc21 ; c1ccc(-n2c3ccccc3c3ccc(-c4ccc(N(c5ccc(-c6cccc7c6ccc6ccccc67)cc5)c5cccc6c5-c5ccccc5C6(c5ccccc5)c5ccccc5)cc4)cc32)cc1 ; CC1(C)c2ccccc2-c2cc(-c3ccc(N(c4ccc(-c5ccc6c7ccccc7n(-c7ccccc7)c6c5)cc4)c4ccc(-c5cccc6c5ccc5ccccc56)cc4)cc3)ccc21]

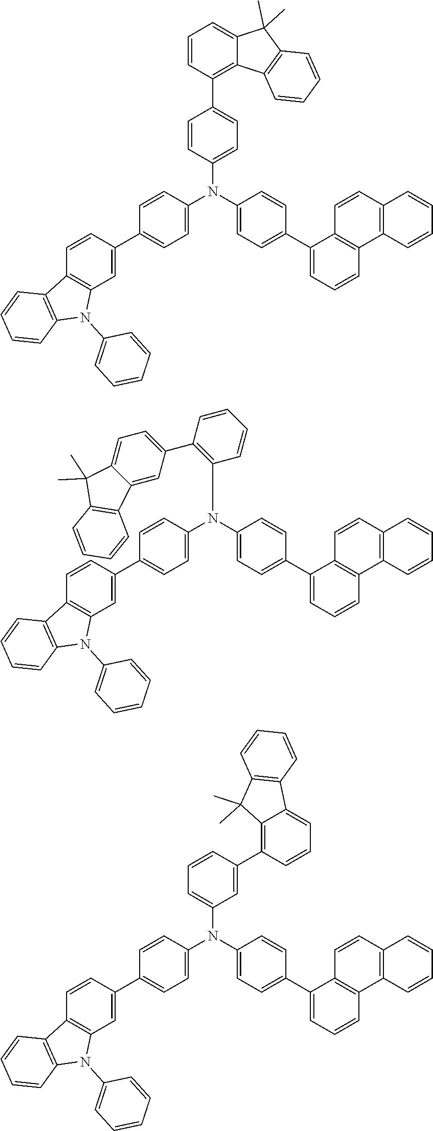 [SMILES: CC1(C)c2ccccc2-c2c(-c3ccc(N(c4ccc(-c5ccc6c7ccccc7n(-c7ccccc7)c6c5)cc4)c4ccc(-c5cccc6c5ccc5ccccc56)cc4)cc3)cccc21 ; CC1(C)c2ccccc2-c2cc(-c3ccccc3N(c3ccc(-c4ccc5c6ccccc6n(-c6ccccc6)c5c4)cc3)c3ccc(-c4cccc5c4ccc4ccccc45)cc3)ccc21 ; CC1(C)c2ccccc2-c2cccc(-c3cccc(N(c4ccc(-c5ccc6c7ccccc7n(-c7ccccc7)c6c5)cc4)c4ccc(-c5cccc6c5ccc5ccccc56)cc4)c3)c21]

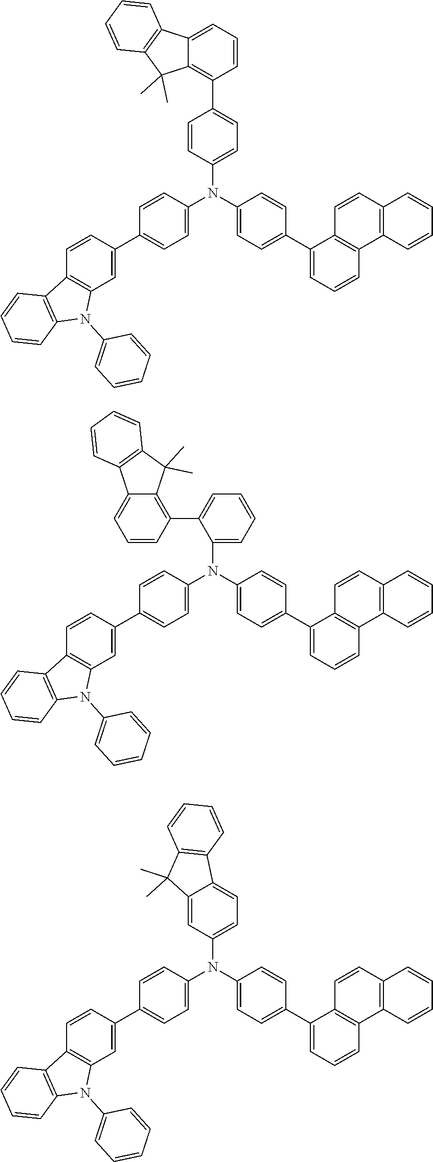

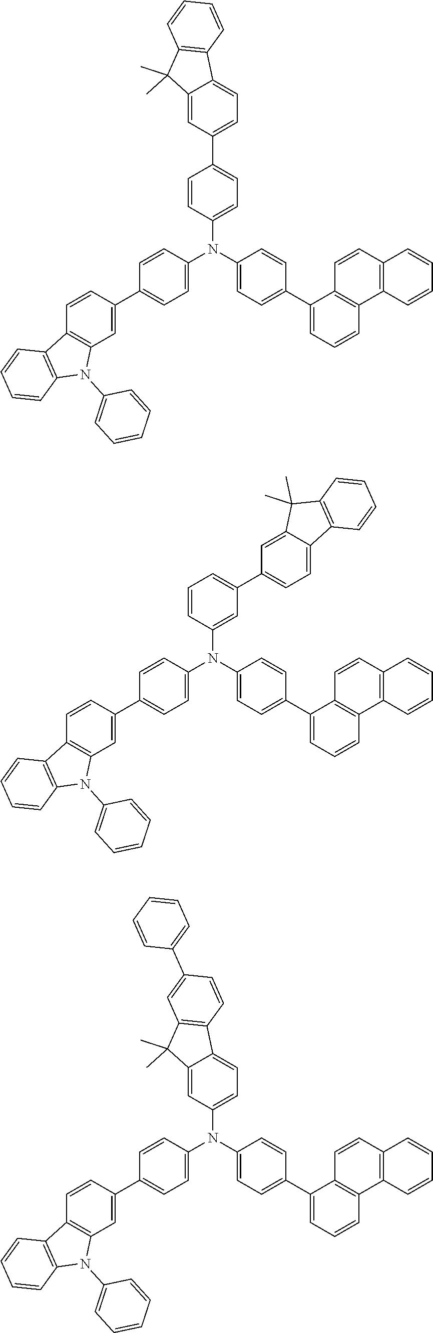 [SMILES: CC1(C)c2ccccc2-c2ccc(-c3ccc(N(c4ccc(-c5ccc6c7ccccc7n(-c7ccccc7)c6c5)cc4)c4ccc(-c5cccc6c5ccc5ccccc56)cc4)cc3)cc21 ; CC1(C)c2ccccc2-c2ccc(-c3cccc(N(c4ccc(-c5ccc6c7ccccc7n(-c7ccccc7)c6c5)cc4)c4ccc(-c5cccc6c5ccc5ccccc56)cc4)c3)cc21 ; CC1(C)c2cc(-c3ccccc3)ccc2-c2ccc(N(c3ccc(-c4ccc5c6ccccc6n(-c6ccccc6)c5c4)cc3)c3ccc(-c4cccc5c4ccc4ccccc45)cc3)cc21]

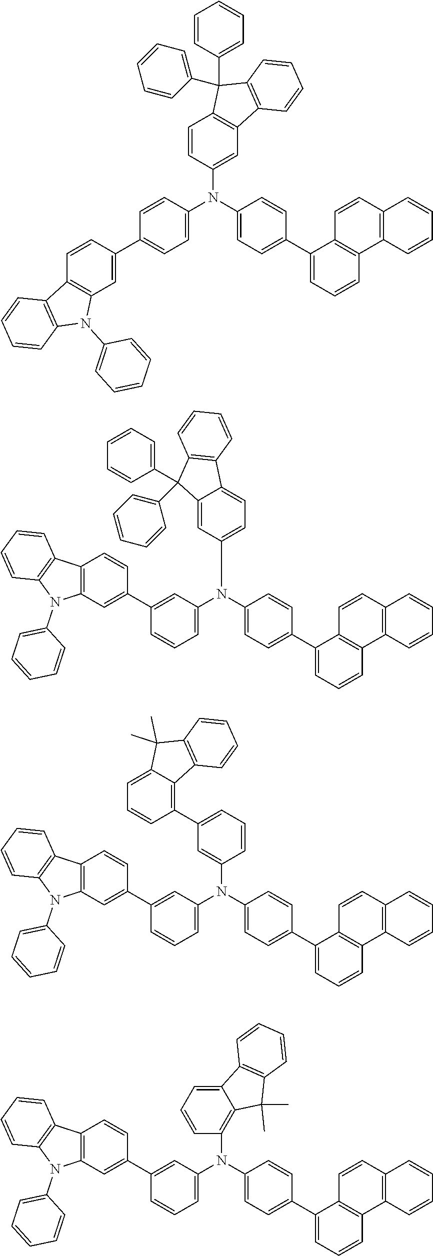 [SMILES: c1ccc(-n2c3ccccc3c3ccc(-c4ccc(N(c5ccc(-c6cccc7c6ccc6ccccc67)cc5)c5ccc6c(c5)-c5ccccc5C6(c5ccccc5)c5ccccc5)cc4)cc32)cc1 ; c1ccc(-n2c3ccccc3c3ccc(-c4cccc(N(c5ccc(-c6cccc7c6ccc6ccccc67)cc5)c5ccc6c(c5)C(c5ccccc5)(c5ccccc5)c5ccccc5-6)c4)cc32)cc1 ; CC1(C)c2ccccc2-c2c(-c3cccc(N(c4ccc(-c5cccc6c5ccc5ccccc56)cc4)c4cccc(-c5ccc6c7ccccc7n(-c7ccccc7)c6c5)c4)c3)cccc21 ; CC1(C)c2ccccc2-c2cccc(N(c3ccc(-c4cccc5c4ccc4ccccc45)cc3)c3cccc(-c4ccc5c6ccccc6n(-c6ccccc6)c5c4)c3)c21]

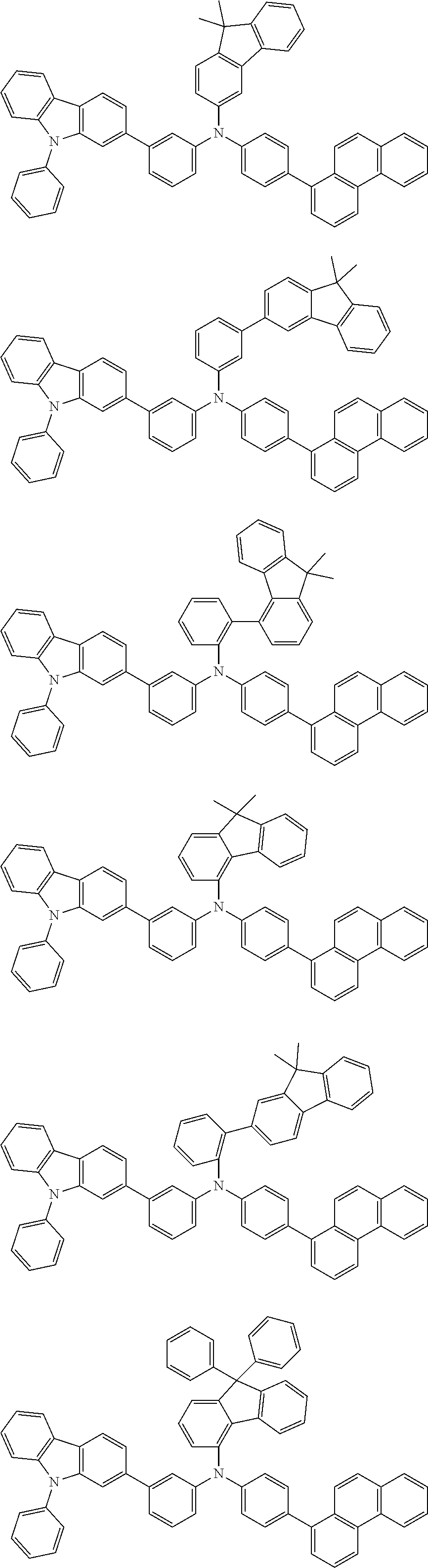

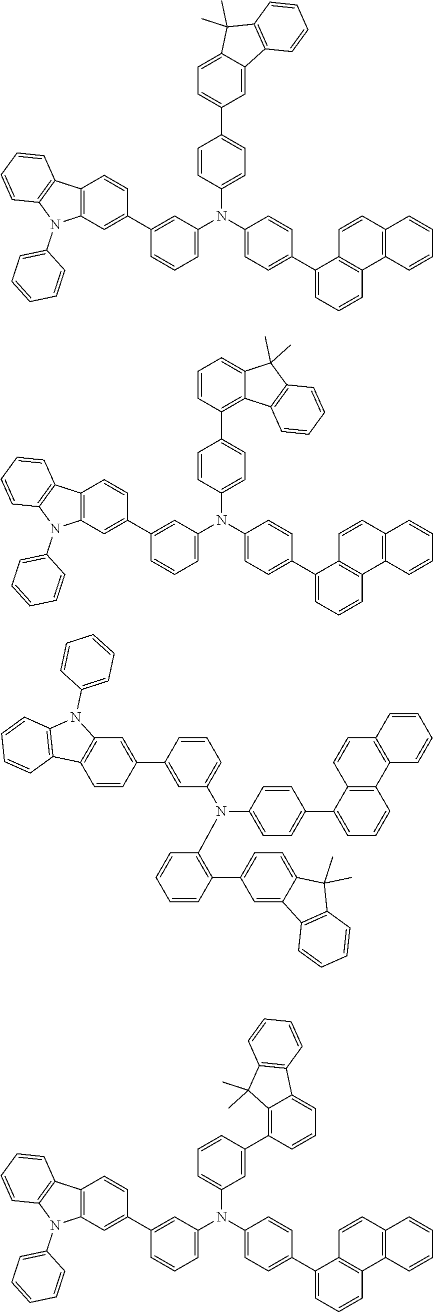 [SMILES: CC1(C)c2ccccc2-c2cc(-c3ccc(N(c4ccc(-c5cccc6c5ccc5ccccc56)cc4)c4cccc(-c5ccc6c7ccccc7n(-c7ccccc7)c6c5)c4)cc3)ccc21 ; CC1(C)c2ccccc2-c2c(-c3ccc(N(c4ccc(-c5cccc6c5ccc5ccccc56)cc4)c4cccc(-c5ccc6c7ccccc7n(-c7ccccc7)c6c5)c4)cc3)cccc21 ; CC1(C)c2ccccc2-c2cc(-c3ccccc3N(c3ccc(-c4cccc5c4ccc4ccccc45)cc3)c3cccc(-c4ccc5c6ccccc6n(-c6ccccc6)c5c4)c3)ccc21 ; CC1(C)c2ccccc2-c2cccc(-c3cccc(N(c4ccc(-c5cccc6c5ccc5ccccc56)cc4)c4cccc(-c5ccc6c7ccccc7n(-c7ccccc7)c6c5)c4)c3)c21]

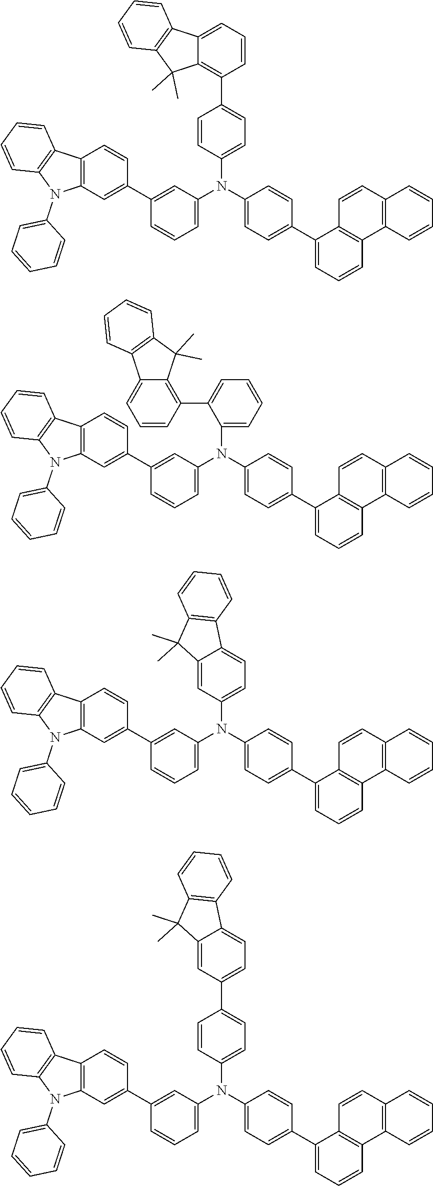 [SMILES: CC1(C)c2ccccc2-c2cccc(-c3ccc(N(c4ccc(-c5cccc6c5ccc5ccccc56)cc4)c4cccc(-c5ccc6c7ccccc7n(-c7ccccc7)c6c5)c4)cc3)c21 ; CC1(C)c2ccccc2-c2cccc(-c3ccccc3N(c3ccc(-c4cccc5c4ccc4ccccc45)cc3)c3cccc(-c4ccc5c6ccccc6n(-c6ccccc6)c5c4)c3)c21 ; CC1(C)c2ccccc2-c2ccc(N(c3ccc(-c4cccc5c4ccc4ccccc45)cc3)c3cccc(-c4ccc5c6ccccc6n(-c6ccccc6)c5c4)c3)cc21 ; CC1(C)c2ccccc2-c2ccc(-c3ccc(N(c4ccc(-c5cccc6c5ccc5ccccc56)cc4)c4cccc(-c5ccc6c7ccccc7n(-c7ccccc7)c6c5)c4)cc3)cc21]

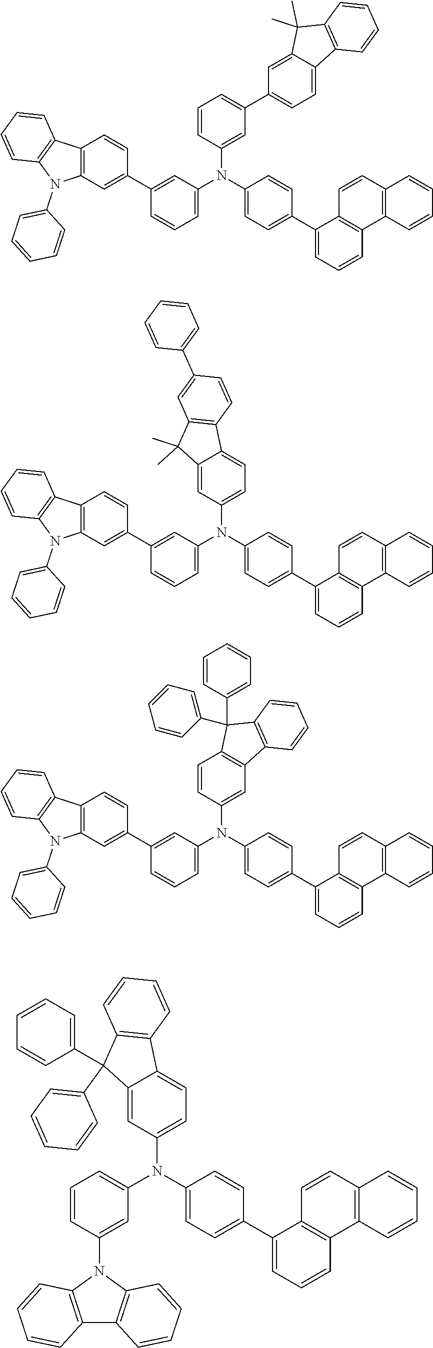 [SMILES: CC1(C)c2ccccc2-c2ccc(-c3cccc(N(c4ccc(-c5cccc6c5ccc5ccccc56)cc4)c4cccc(-c5ccc6c7ccccc7n(-c7ccccc7)c6c5)c4)c3)cc21 ; CC1(C)c2cc(-c3ccccc3)ccc2-c2ccc(N(c3ccc(-c4cccc5c4ccc4ccccc45)cc3)c3cccc(-c4ccc5c6ccccc6n(-c6ccccc6)c5c4)c3)cc21 ; c1ccc(-n2c3ccccc3c3ccc(-c4cccc(N(c5ccc(-c6cccc7c6ccc6ccccc67)cc5)c5ccc6c(c5)-c5ccccc5C6(c5ccccc5)c5ccccc5)c4)cc32)cc1 ; c1ccc(C2(c3ccccc3)c3ccccc3-c3ccc(N(c4ccc(-c5cccc6c5ccc5ccccc56)cc4)c4cccc(-n5c6ccccc6c6ccccc65)c4)cc32)cc1]

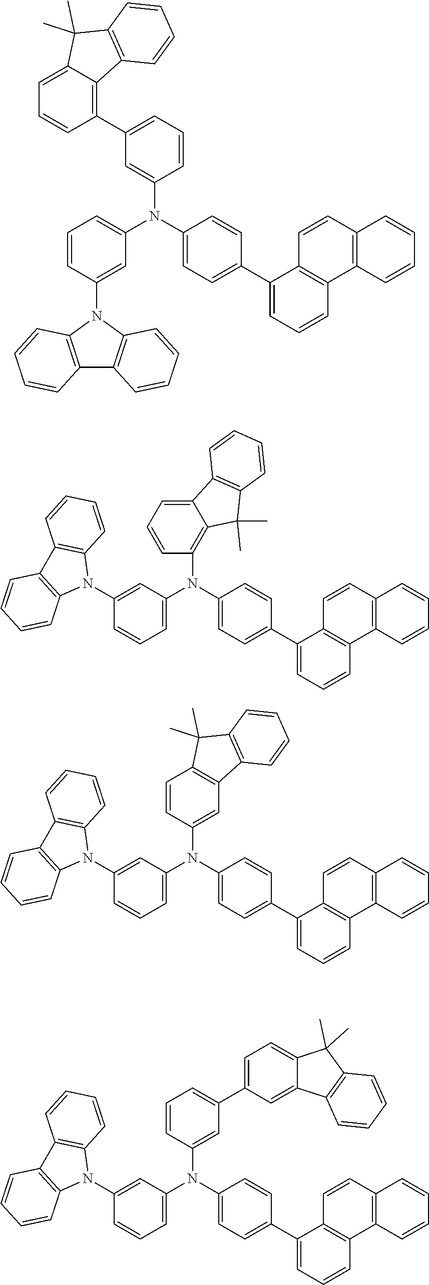 [SMILES: CC1(C)c2ccccc2-c2c(-c3cccc(N(c4ccc(-c5cccc6c5ccc5ccccc56)cc4)c4cccc(-n5c6ccccc6c6ccccc65)c4)c3)cccc21 ; CC1(C)c2ccccc2-c2cccc(N(c3ccc(-c4cccc5c4ccc4ccccc45)cc3)c3cccc(-n4c5ccccc5c5ccccc54)c3)c21 ; CC1(C)c2ccccc2-c2cc(N(c3ccc(-c4cccc5c4ccc4ccccc45)cc3)c3cccc(-n4c5ccccc5c5ccccc54)c3)ccc21 ; CC1(C)c2ccccc2-c2cc(-c3cccc(N(c4ccc(-c5cccc6c5ccc5ccccc56)cc4)c4cccc(-n5c6ccccc6c6ccccc65)c4)c3)ccc21]

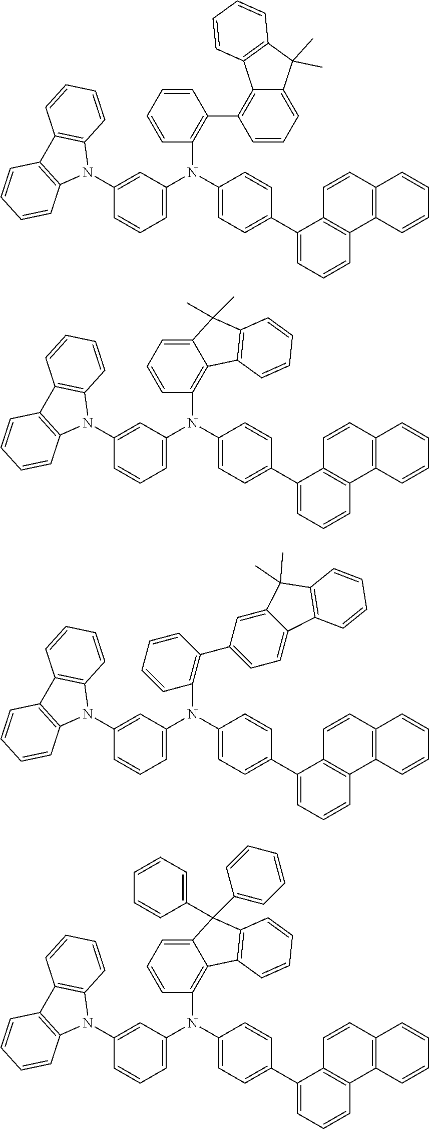

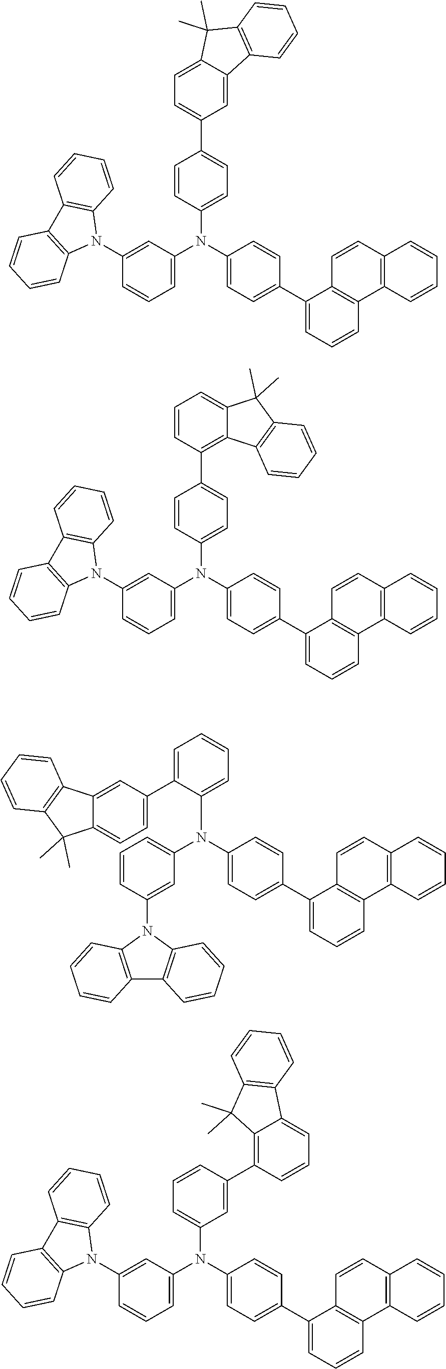 [SMILES: CC1(C)c2ccccc2-c2cc(-c3ccc(N(c4ccc(-c5cccc6c5ccc5ccccc56)cc4)c4cccc(-n5c6ccccc6c6ccccc65)c4)cc3)ccc21 ; CC1(C)c2ccccc2-c2c(-c3ccc(N(c4ccc(-c5cccc6c5ccc5ccccc56)cc4)c4cccc(-n5c6ccccc6c6ccccc65)c4)cc3)cccc21 ; CC1(C)c2ccccc2-c2cc(-c3ccccc3N(c3ccc(-c4cccc5c4ccc4ccccc45)cc3)c3cccc(-n4c5ccccc5c5ccccc54)c3)ccc21 ; CC1(C)c2ccccc2-c2cccc(-c3cccc(N(c4ccc(-c5cccc6c5ccc5ccccc56)cc4)c4cccc(-n5c6ccccc6c6ccccc65)c4)c3)c21]

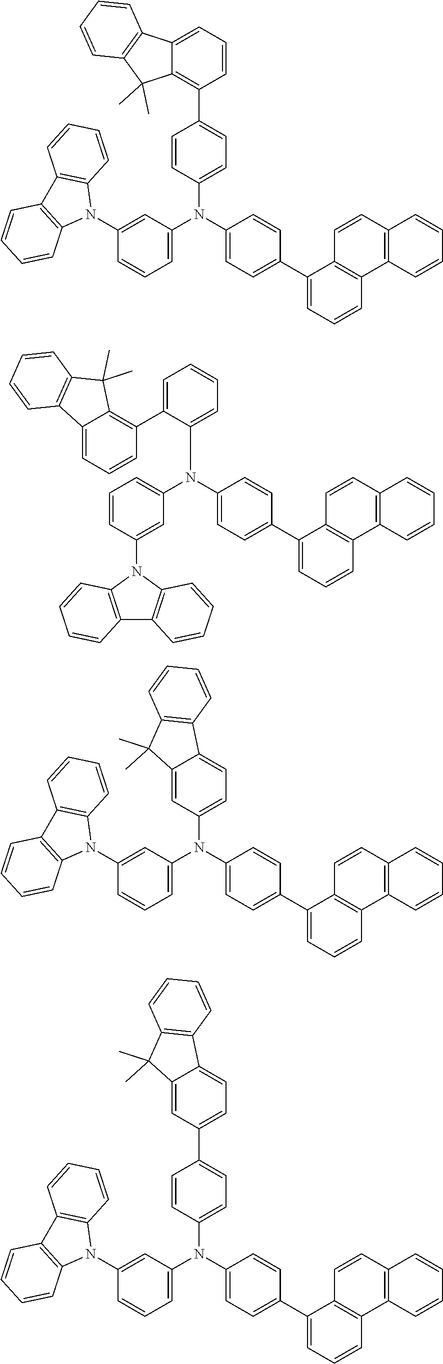

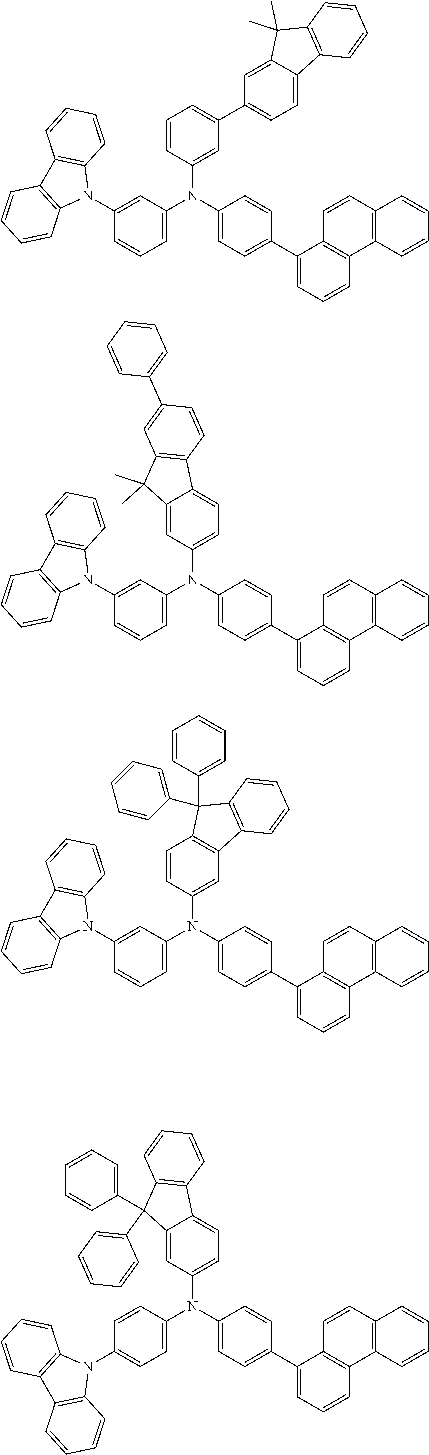 [SMILES: CC1(C)c2ccccc2-c2ccc(-c3cccc(N(c4ccc(-c5cccc6c5ccc5ccccc56)cc4)c4cccc(-n5c6ccccc6c6ccccc65)c4)c3)cc21 ; CC1(C)c2cc(-c3ccccc3)ccc2-c2ccc(N(c3ccc(-c4cccc5c4ccc4ccccc45)cc3)c3cccc(-n4c5ccccc5c5ccccc54)c3)cc21 ; c1ccc(C2(c3ccccc3)c3ccccc3-c3cc(N(c4ccc(-c5cccc6c5ccc5ccccc56)cc4)c4cccc(-n5c6ccccc6c6ccccc65)c4)ccc32)cc1 ; c1ccc(C2(c3ccccc3)c3ccccc3-c3ccc(N(c4ccc(-c5cccc6c5ccc5ccccc56)cc4)c4ccc(-n5c6ccccc6c6ccccc65)cc4)cc32)cc1]

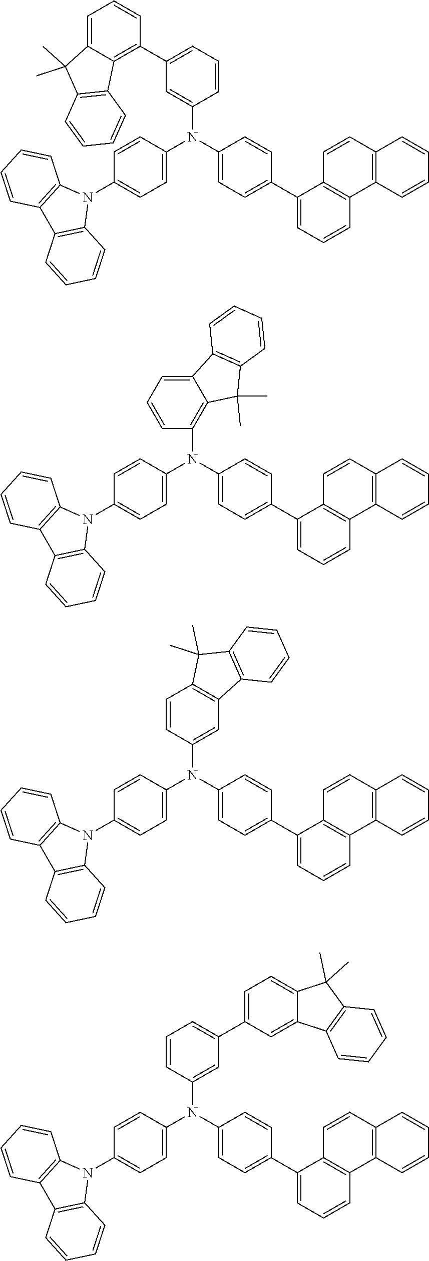 [SMILES: CC1(C)c2ccccc2-c2c(-c3cccc(N(c4ccc(-c5cccc6c5ccc5ccccc56)cc4)c4ccc(-n5c6ccccc6c6ccccc65)cc4)c3)cccc21 ; CC1(C)c2ccccc2-c2cccc(N(c3ccc(-c4cccc5c4ccc4ccccc45)cc3)c3ccc(-n4c5ccccc5c5ccccc54)cc3)c21 ; CC1(C)c2ccccc2-c2cc(N(c3ccc(-c4cccc5c4ccc4ccccc45)cc3)c3ccc(-n4c5ccccc5c5ccccc54)cc3)ccc21 ; CC1(C)c2ccccc2-c2cc(-c3cccc(N(c4ccc(-c5cccc6c5ccc5ccccc56)cc4)c4ccc(-n5c6ccccc6c6ccccc65)cc4)c3)ccc21]

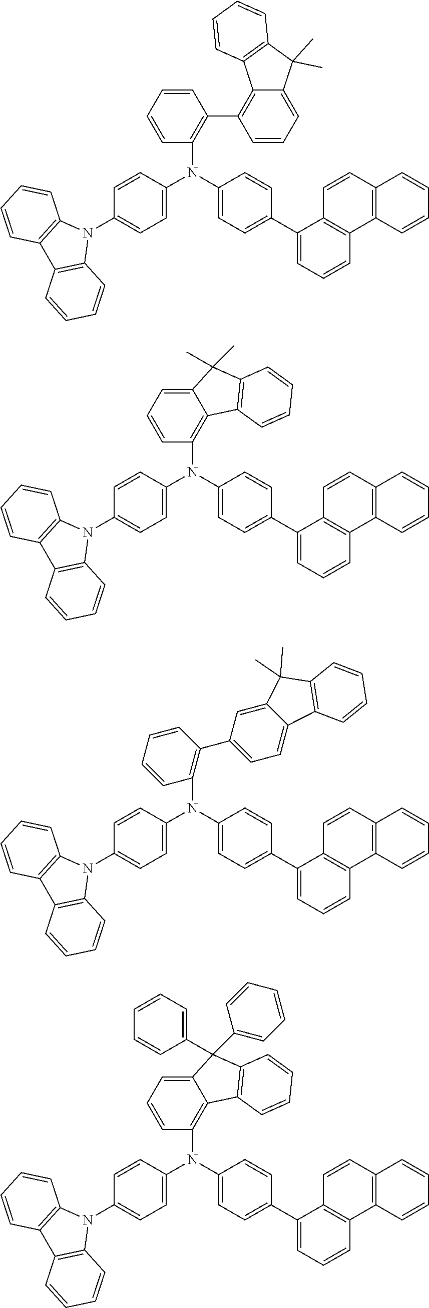 [SMILES: CC1(C)c2ccccc2-c2c(-c3ccccc3N(c3ccc(-c4cccc5c4ccc4ccccc45)cc3)c3ccc(-n4c5ccccc5c5ccccc54)cc3)cccc21 ; CC1(C)c2ccccc2-c2c(N(c3ccc(-c4cccc5c4ccc4ccccc45)cc3)c3ccc(-n4c5ccccc5c5ccccc54)cc3)cccc21 ; CC1(C)c2ccccc2-c2ccc(-c3ccccc3N(c3ccc(-c4cccc5c4ccc4ccccc45)cc3)c3ccc(-n4c5ccccc5c5ccccc54)cc3)cc21 ; c1ccc(C2(c3ccccc3)c3ccccc3-c3c(N(c4ccc(-c5cccc6c5ccc5ccccc56)cc4)c4ccc(-n5c6ccccc6c6ccccc65)cc4)cccc32)cc1]

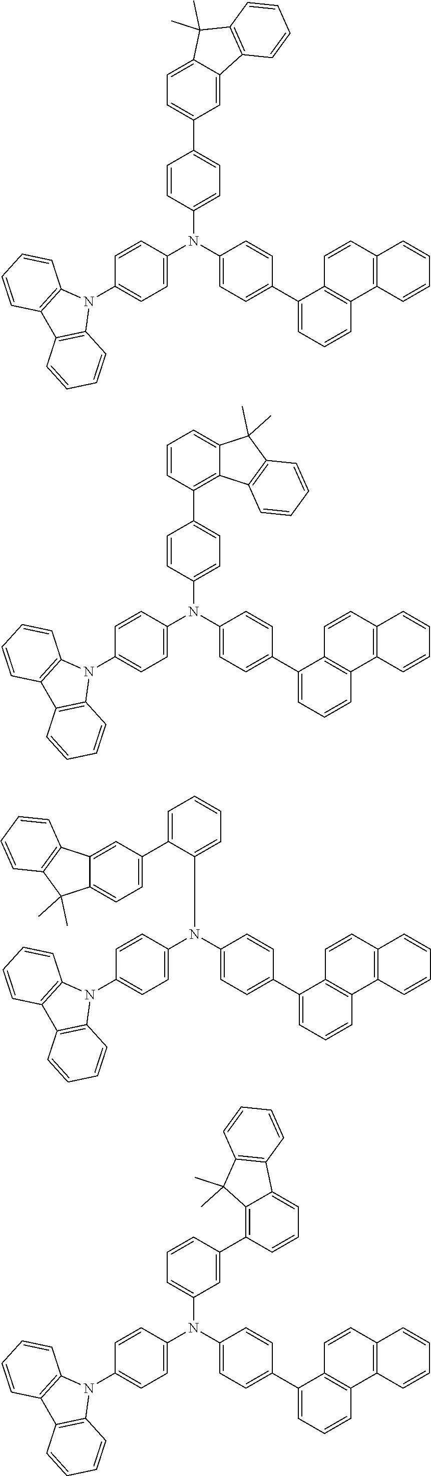 [SMILES: CC1(C)c2ccccc2-c2cc(-c3ccc(N(c4ccc(-c5cccc6c5ccc5ccccc56)cc4)c4ccc(-n5c6ccccc6c6ccccc65)cc4)cc3)ccc21 ; CC1(C)c2ccccc2-c2c(-c3ccc(N(c4ccc(-c5cccc6c5ccc5ccccc56)cc4)c4ccc(-n5c6ccccc6c6ccccc65)cc4)cc3)cccc21 ; CC1(C)c2ccccc2-c2cc(-c3ccccc3N(c3ccc(-c4cccc5c4ccc4ccccc45)cc3)c3ccc(-n4c5ccccc5c5ccccc54)cc3)ccc21 ; CC1(C)c2ccccc2-c2cccc(-c3cccc(N(c4ccc(-c5cccc6c5ccc5ccccc56)cc4)c4ccc(-n5c6ccccc6c6ccccc65)cc4)c3)c21]

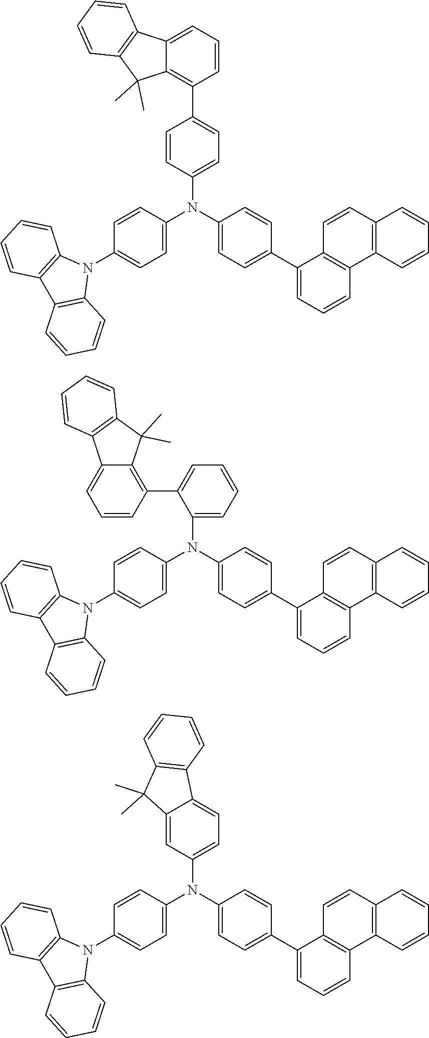

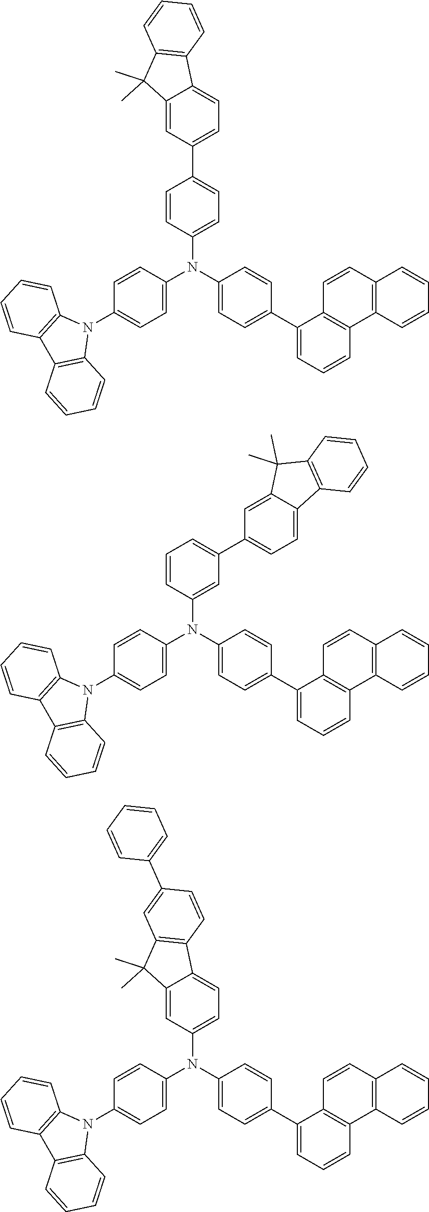 [SMILES: CC1(C)c2ccccc2-c2ccc(-c3ccc(N(c4ccc(-c5cccc6c5ccc5ccccc56)cc4)c4ccc(-n5c6ccccc6c6ccccc65)cc4)cc3)cc21 ; CC1(C)c2ccccc2-c2ccc(-c3cccc(N(c4ccc(-c5cccc6c5ccc5ccccc56)cc4)c4ccc(-n5c6ccccc6c6ccccc65)cc4)c3)cc21 ; CC1(C)c2cc(-c3ccccc3)ccc2-c2ccc(N(c3ccc(-c4cccc5c4ccc4ccccc45)cc3)c3ccc(-n4c5ccccc5c5ccccc54)cc3)cc21]

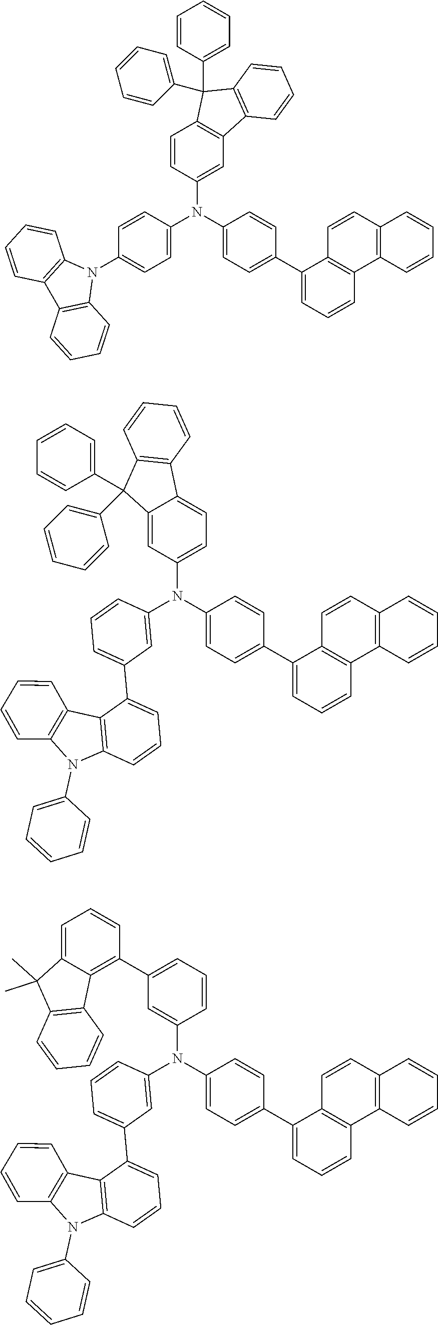 [SMILES: c1ccc(C2(c3ccccc3)c3ccccc3-c3cc(N(c4ccc(-c5cccc6c5ccc5ccccc56)cc4)c4ccc(-n5c6ccccc6c6ccccc65)cc4)ccc32)cc1 ; c1ccc(-n2c3ccccc3c3c(-c4cccc(N(c5ccc(-c6cccc7c6ccc6ccccc67)cc5)c5ccc6c(c5)C(c5ccccc5)(c5ccccc5)c5ccccc5-6)c4)cccc32)cc1 ; CC1(C)c2ccccc2-c2c(-c3cccc(N(c4ccc(-c5cccc6c5ccc5ccccc56)cc4)c4cccc(-c5cccc6c5c5ccccc5n6-c5ccccc5)c4)c3)cccc21]

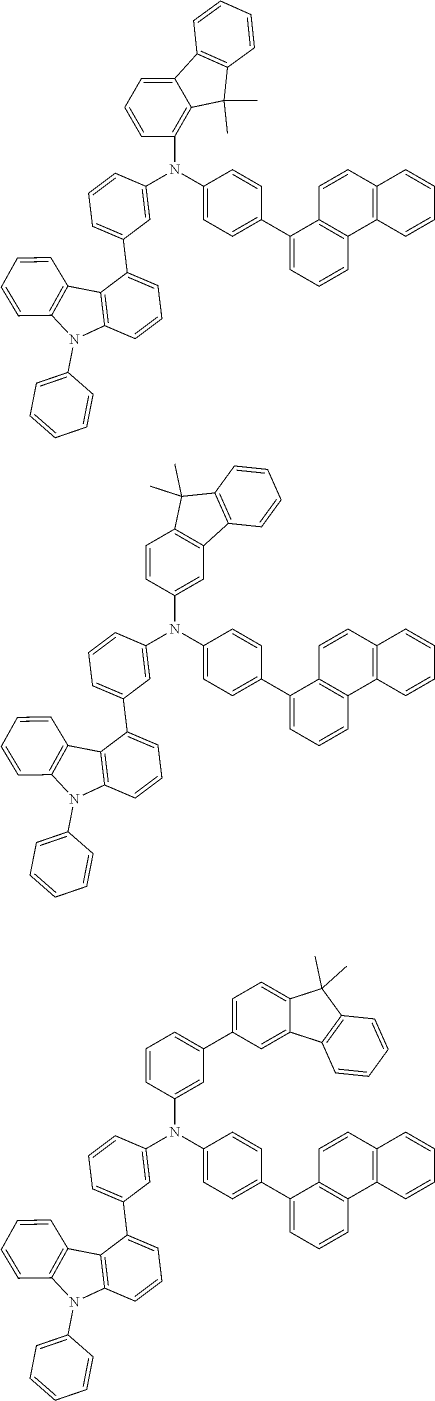 [SMILES: CC1(C)c2ccccc2-c2cccc(N(c3ccc(-c4cccc5c4ccc4ccccc45)cc3)c3cccc(-c4cccc5c4c4ccccc4n5-c4ccccc4)c3)c21 ; CC1(C)c2ccccc2-c2cc(N(c3ccc(-c4cccc5c4ccc4ccccc45)cc3)c3cccc(-c4cccc5c4c4ccccc4n5-c4ccccc4)c3)ccc21 ; CC1(C)c2ccccc2-c2cc(-c3cccc(N(c4ccc(-c5cccc6c5ccc5ccccc56)cc4)c4cccc(-c5cccc6c5c5ccccc5n6-c5ccccc5)c4)c3)ccc21]

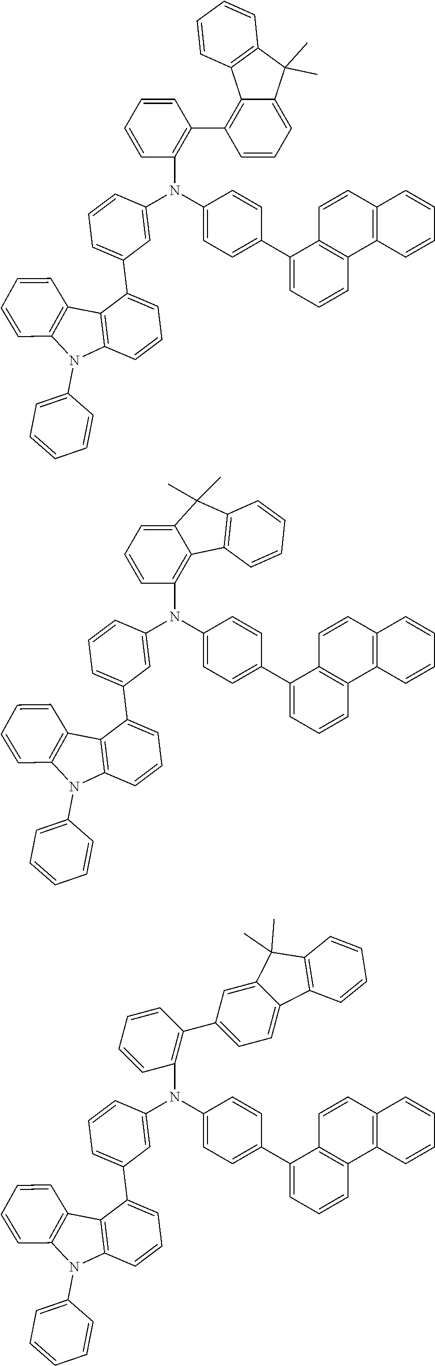 [SMILES: CC1(C)c2ccccc2-c2c(-c3ccccc3N(c3ccc(-c4cccc5c4ccc4ccccc45)cc3)c3cccc(-c4cccc5c4c4ccccc4n5-c4ccccc4)c3)cccc21 ; CC1(C)c2ccccc2-c2c(N(c3ccc(-c4cccc5c4ccc4ccccc45)cc3)c3cccc(-c4cccc5c4c4ccccc4n5-c4ccccc4)c3)cccc21 ; CC1(C)c2ccccc2-c2ccc(-c3ccccc3N(c3ccc(-c4cccc5c4ccc4ccccc45)cc3)c3cccc(-c4cccc5c4c4ccccc4n5-c4ccccc4)c3)cc21]

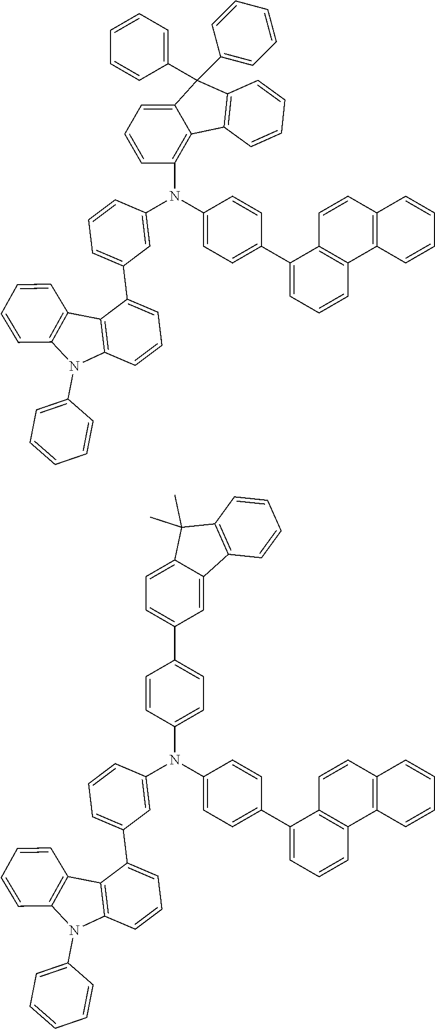 [SMILES: c1ccc(-n2c3ccccc3c3c(-c4cccc(N(c5ccc(-c6cccc7c6ccc6ccccc67)cc5)c5cccc6c5-c5ccccc5C6(c5ccccc5)c5ccccc5)c4)cccc32)cc1 ; CC1(C)c2ccccc2-c2cc(-c3ccc(N(c4ccc(-c5cccc6c5ccc5ccccc56)cc4)c4cccc(-c5cccc6c5c5ccccc5n6-c5ccccc5)c4)cc3)ccc21]

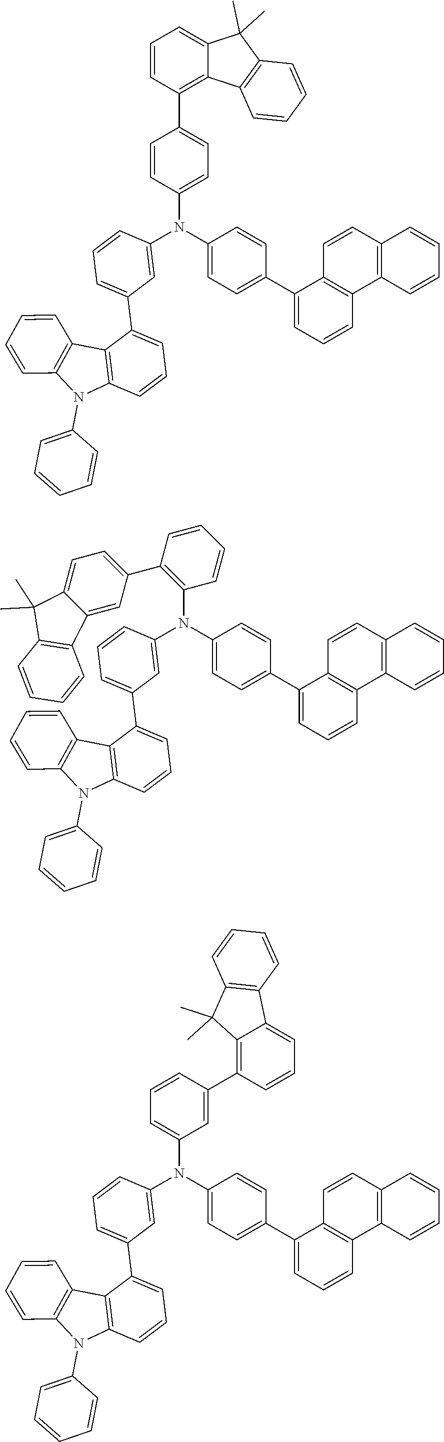 [SMILES: CC1(C)c2ccccc2-c2c(-c3ccc(N(c4ccc(-c5cccc6c5ccc5ccccc56)cc4)c4cccc(-c5cccc6c5c5ccccc5n6-c5ccccc5)c4)cc3)cccc21 ; CC1(C)c2ccccc2-c2cc(-c3ccccc3N(c3ccc(-c4cccc5c4ccc4ccccc45)cc3)c3cccc(-c4cccc5c4c4ccccc4n5-c4ccccc4)c3)ccc21 ; CC1(C)c2ccccc2-c2cccc(-c3cccc(N(c4ccc(-c5cccc6c5ccc5ccccc56)cc4)c4cccc(-c5cccc6c5c5ccccc5n6-c5ccccc5)c4)c3)c21]

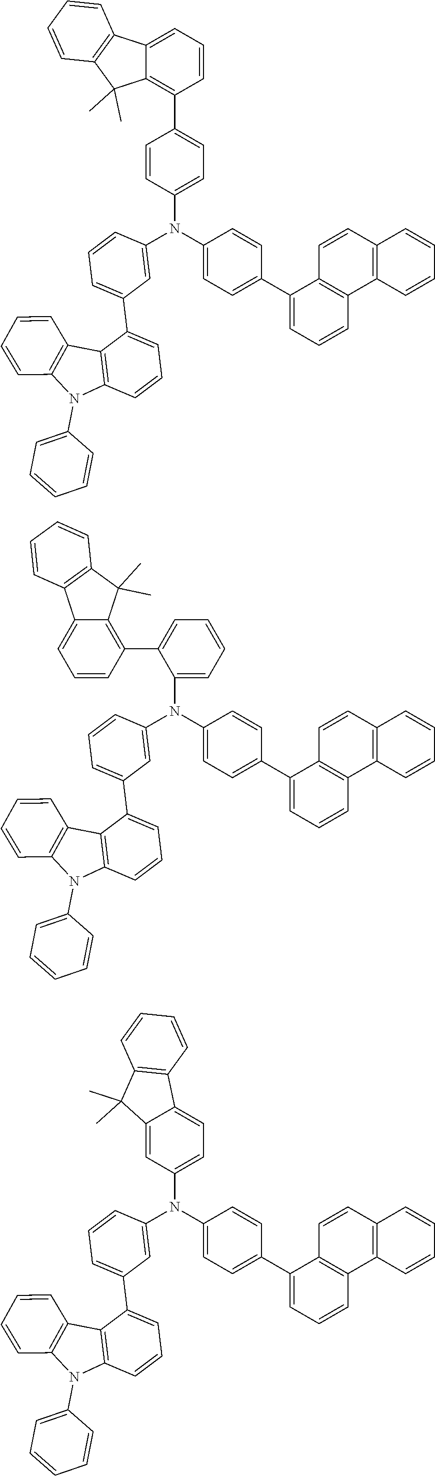

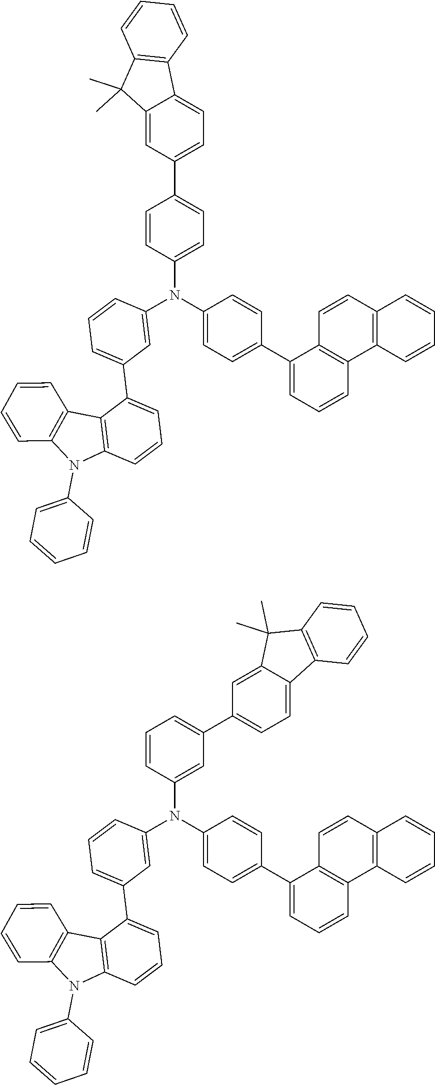 [SMILES: CC1(C)c2ccccc2-c2ccc(-c3ccc(N(c4ccc(-c5cccc6c5ccc5ccccc56)cc4)c4cccc(-c5cccc6c5c5ccccc5n6-c5ccccc5)c4)cc3)cc21 ; CC1(C)c2ccccc2-c2ccc(-c3cccc(N(c4ccc(-c5cccc6c5ccc5ccccc56)cc4)c4cccc(-c5cccc6c5c5ccccc5n6-c5ccccc5)c4)c3)cc21]

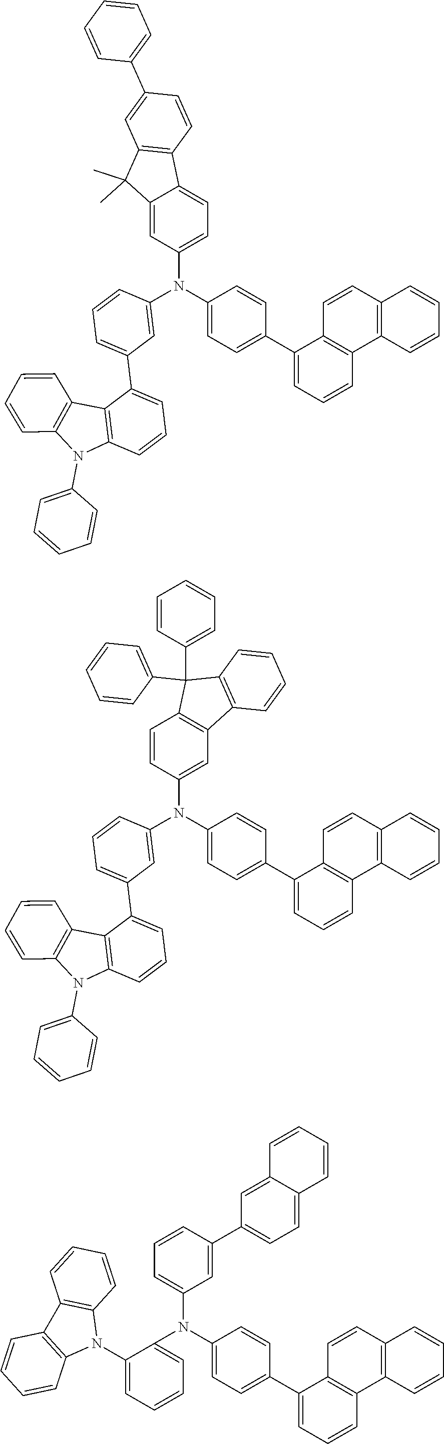 [SMILES: CC1(C)c2cc(-c3ccccc3)ccc2-c2ccc(N(c3ccc(-c4cccc5c4ccc4ccccc45)cc3)c3cccc(-c4cccc5c4c4ccccc4n5-c4ccccc4)c3)cc21 ; c1ccc(-n2c3ccccc3c3c(-c4cccc(N(c5ccc(-c6cccc7c6ccc6ccccc67)cc5)c5ccc6c(c5)-c5ccccc5C6(c5ccccc5)c5ccccc5)c4)cccc32)cc1 ; c1cc(-c2ccc3ccccc3c2)cc(N(c2ccc(-c3cccc4c3ccc3ccccc34)cc2)c2ccccc2-n2c3ccccc3c3ccccc32)c1]

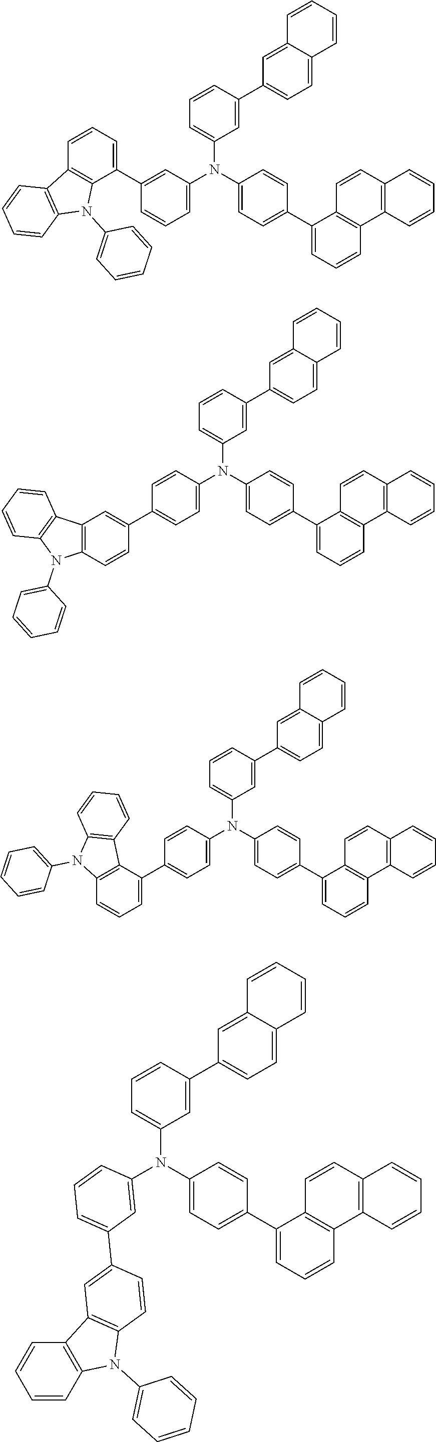 [SMILES: c1ccc(-n2c3ccccc3c3cccc(-c4cccc(N(c5ccc(-c6cccc7c6ccc6ccccc67)cc5)c5cccc(-c6ccc7ccccc7c6)c5)c4)c32)cc1 ; c1ccc(-n2c3ccccc3c3cc(-c4ccc(N(c5ccc(-c6cccc7c6ccc6ccccc67)cc5)c5cccc(-c6ccc7ccccc7c6)c5)cc4)ccc32)cc1 ; c1ccc(-n2c3ccccc3c3c(-c4ccc(N(c5ccc(-c6cccc7c6ccc6ccccc67)cc5)c5cccc(-c6ccc7ccccc7c6)c5)cc4)cccc32)cc1 ; c1ccc(-n2c3ccccc3c3cc(-c4cccc(N(c5ccc(-c6cccc7c6ccc6ccccc67)cc5)c5cccc(-c6ccc7ccccc7c6)c5)c4)ccc32)cc1]

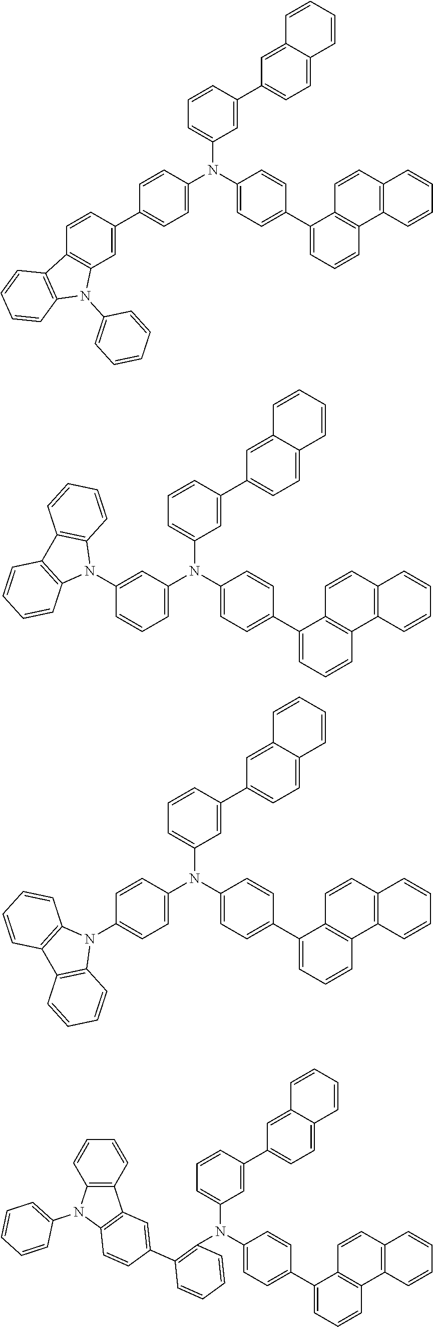

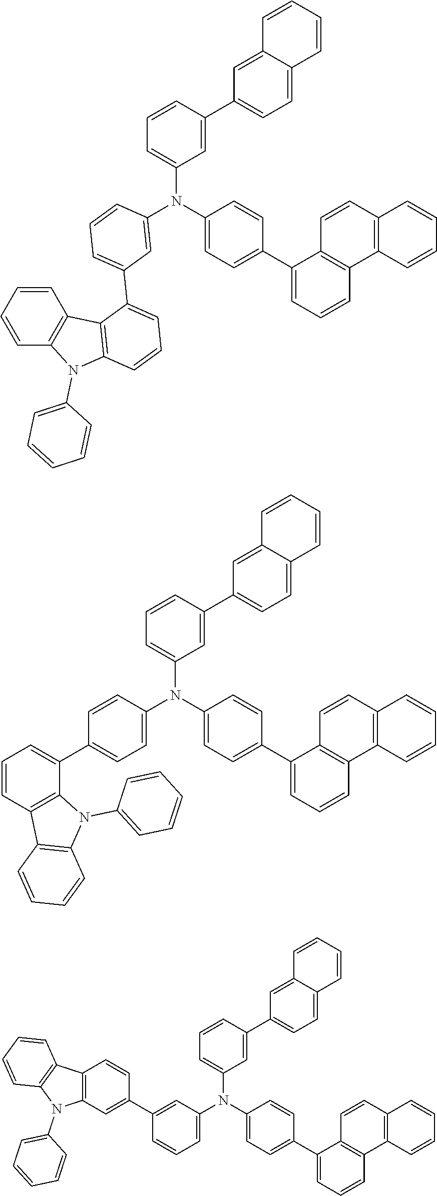 [SMILES: c1ccc(-n2c3ccccc3c3c(-c4cccc(N(c5ccc(-c6cccc7c6ccc6ccccc67)cc5)c5cccc(-c6ccc7ccccc7c6)c5)c4)cccc32)cc1 ; c1ccc(-n2c3ccccc3c3cccc(-c4ccc(N(c5ccc(-c6cccc7c6ccc6ccccc67)cc5)c5cccc(-c6ccc7ccccc7c6)c5)cc4)c32)cc1 ; c1ccc(-n2c3ccccc3c3ccc(-c4cccc(N(c5ccc(-c6cccc7c6ccc6ccccc67)cc5)c5cccc(-c6ccc7ccccc7c6)c5)c4)cc32)cc1]

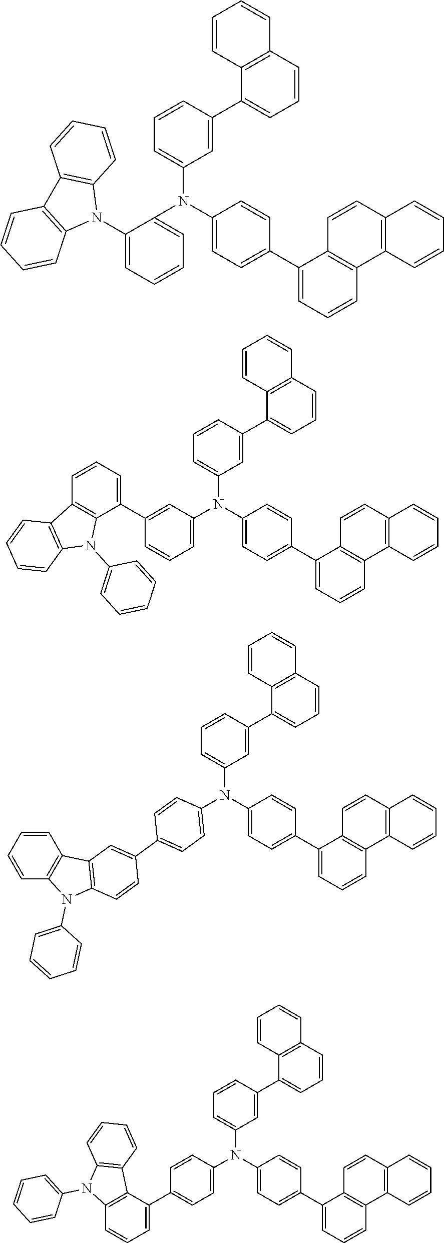 [SMILES: c1cc(-c2cccc3ccccc23)cc(N(c2ccc(-c3cccc4c3ccc3ccccc34)cc2)c2ccccc2-n2c3ccccc3c3ccccc32)c1 ; c1ccc(-n2c3ccccc3c3cccc(-c4cccc(N(c5ccc(-c6cccc7c6ccc6ccccc67)cc5)c5cccc(-c6cccc7ccccc67)c5)c4)c32)cc1 ; c1ccc(-n2c3ccccc3c3cc(-c4ccc(N(c5ccc(-c6cccc7c6ccc6ccccc67)cc5)c5cccc(-c6cccc7ccccc67)c5)cc4)ccc32)cc1 ; c1ccc(-n2c3ccccc3c3c(-c4ccc(N(c5ccc(-c6cccc7c6ccc6ccccc67)cc5)c5cccc(-c6cccc7ccccc67)c5)cc4)cccc32)cc1]

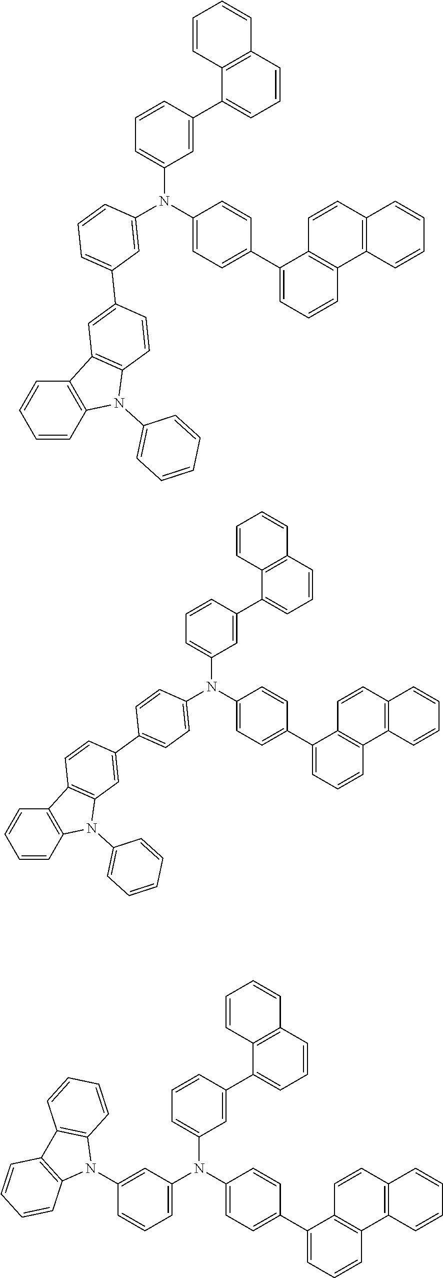 [SMILES: c1ccc(-n2c3ccccc3c3cc(-c4cccc(N(c5ccc(-c6cccc7c6ccc6ccccc67)cc5)c5cccc(-c6cccc7ccccc67)c5)c4)ccc32)cc1 ; c1ccc(-n2c3ccccc3c3ccc(-c4ccc(N(c5ccc(-c6cccc7c6ccc6ccccc67)cc5)c5cccc(-c6cccc7ccccc67)c5)cc4)cc32)cc1 ; c1cc(-c2cccc3ccccc23)cc(N(c2ccc(-c3cccc4c3ccc3ccccc34)cc2)c2cccc(-n3c4ccccc4c4ccccc43)c2)c1]

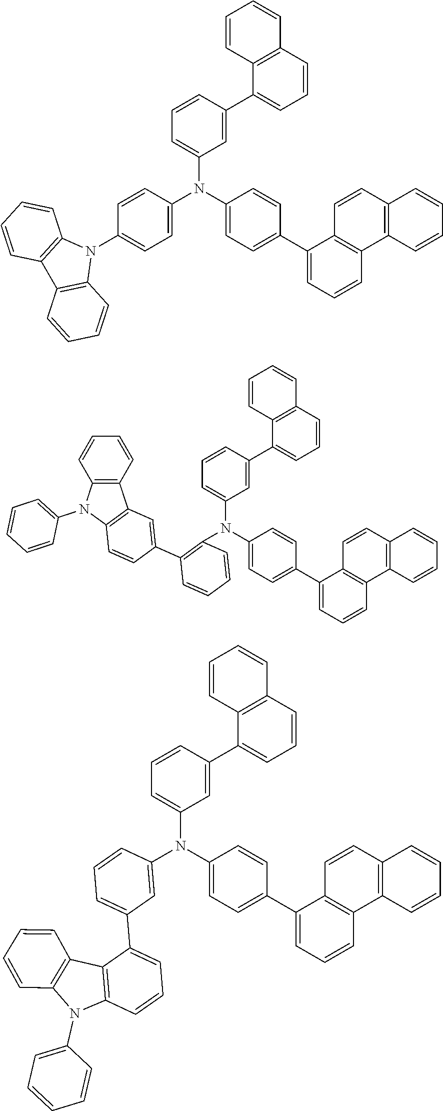 [SMILES: c1cc(-c2cccc3ccccc23)cc(N(c2ccc(-c3cccc4c3ccc3ccccc34)cc2)c2ccc(-n3c4ccccc4c4ccccc43)cc2)c1 ; c1ccc(-n2c3ccccc3c3cc(-c4ccccc4N(c4ccc(-c5cccc6c5ccc5ccccc56)cc4)c4cccc(-c5cccc6ccccc56)c4)ccc32)cc1 ; c1ccc(-n2c3ccccc3c3c(-c4cccc(N(c5ccc(-c6cccc7c6ccc6ccccc67)cc5)c5cccc(-c6cccc7ccccc67)c5)c4)cccc32)cc1]

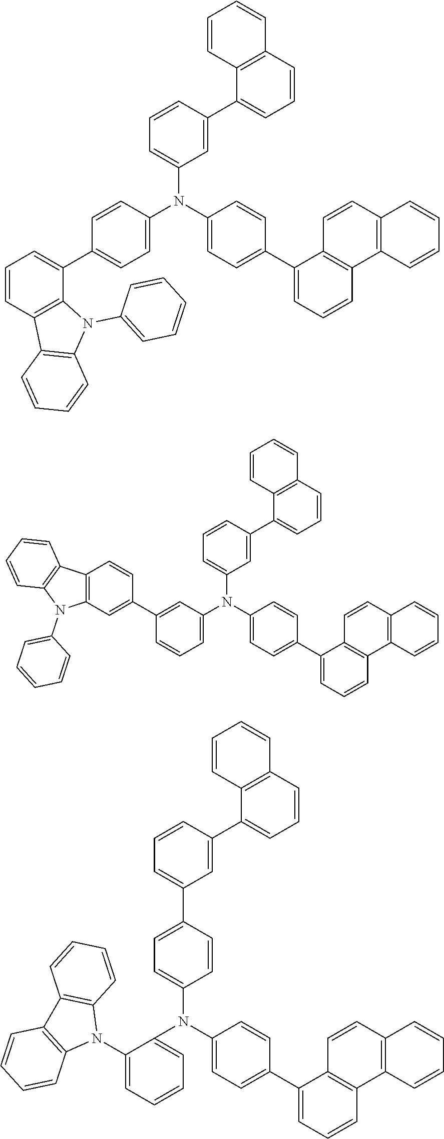 [SMILES: c1ccc(-n2c3ccccc3c3cccc(-c4ccc(N(c5ccc(-c6cccc7c6ccc6ccccc67)cc5)c5cccc(-c6cccc7ccccc67)c5)cc4)c32)cc1 ; c1ccc(-n2c3ccccc3c3ccc(-c4cccc(N(c5ccc(-c6cccc7c6ccc6ccccc67)cc5)c5cccc(-c6cccc7ccccc67)c5)c4)cc32)cc1 ; c1cc(-c2ccc(N(c3ccc(-c4cccc5c4ccc4ccccc45)cc3)c3ccccc3-n3c4ccccc4c4ccccc43)cc2)cc(-c2cccc3ccccc23)c1]

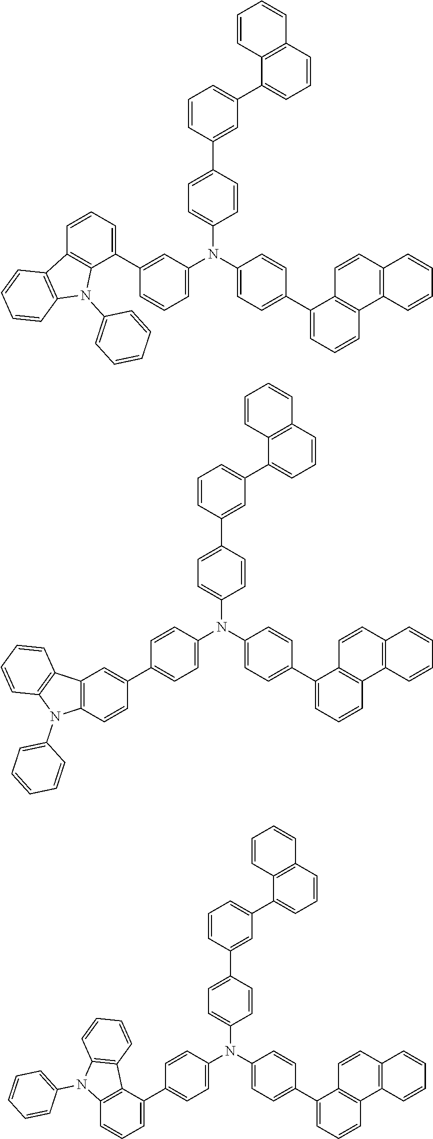 [SMILES: c1ccc(-n2c3ccccc3c3cccc(-c4cccc(N(c5ccc(-c6cccc(-c7cccc8ccccc78)c6)cc5)c5ccc(-c6cccc7c6ccc6ccccc67)cc5)c4)c32)cc1 ; c1ccc(-n2c3ccccc3c3cc(-c4ccc(N(c5ccc(-c6cccc(-c7cccc8ccccc78)c6)cc5)c5ccc(-c6cccc7c6ccc6ccccc67)cc5)cc4)ccc32)cc1 ; c1ccc(-n2c3ccccc3c3c(-c4ccc(N(c5ccc(-c6cccc(-c7cccc8ccccc78)c6)cc5)c5ccc(-c6cccc7c6ccc6ccccc67)cc5)cc4)cccc32)cc1]

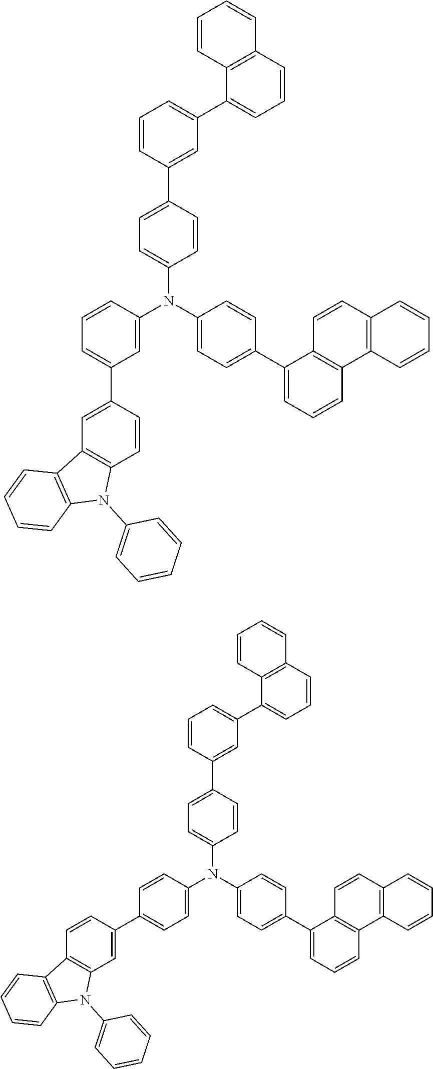 [SMILES: c1ccc(-n2c3ccccc3c3cc(-c4cccc(N(c5ccc(-c6cccc(-c7cccc8ccccc78)c6)cc5)c5ccc(-c6cccc7c6ccc6ccccc67)cc5)c4)ccc32)cc1 ; c1ccc(-n2c3ccccc3c3ccc(-c4ccc(N(c5ccc(-c6cccc(-c7cccc8ccccc78)c6)cc5)c5ccc(-c6cccc7c6ccc6ccccc67)cc5)cc4)cc32)cc1]

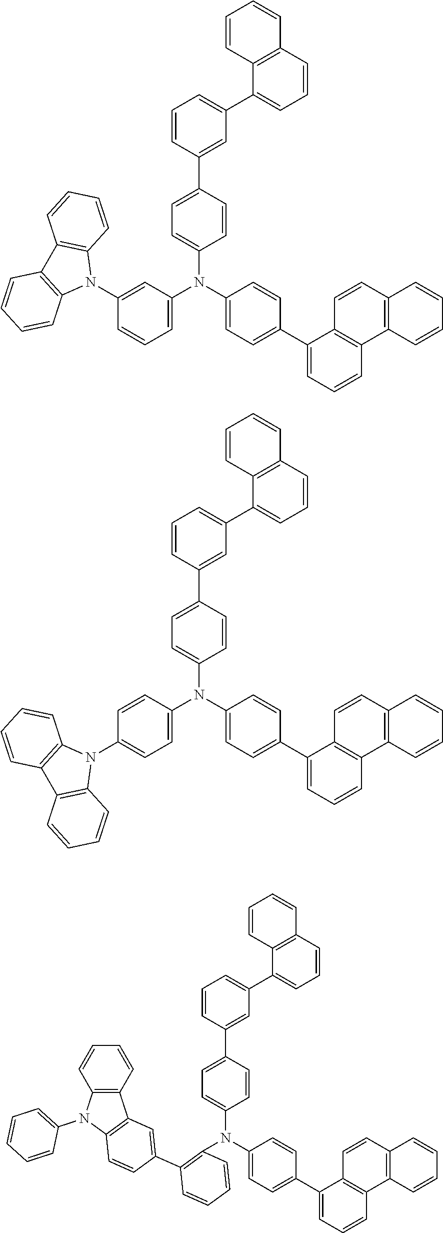 [SMILES: c1cc(-c2ccc(N(c3ccc(-c4cccc5c4ccc4ccccc45)cc3)c3cccc(-n4c5ccccc5c5ccccc54)c3)cc2)cc(-c2cccc3ccccc23)c1 ; c1cc(-c2ccc(N(c3ccc(-c4cccc5c4ccc4ccccc45)cc3)c3ccc(-n4c5ccccc5c5ccccc54)cc3)cc2)cc(-c2cccc3ccccc23)c1 ; c1ccc(-n2c3ccccc3c3cc(-c4ccccc4N(c4ccc(-c5cccc(-c6cccc7ccccc67)c5)cc4)c4ccc(-c5cccc6c5ccc5ccccc56)cc4)ccc32)cc1]

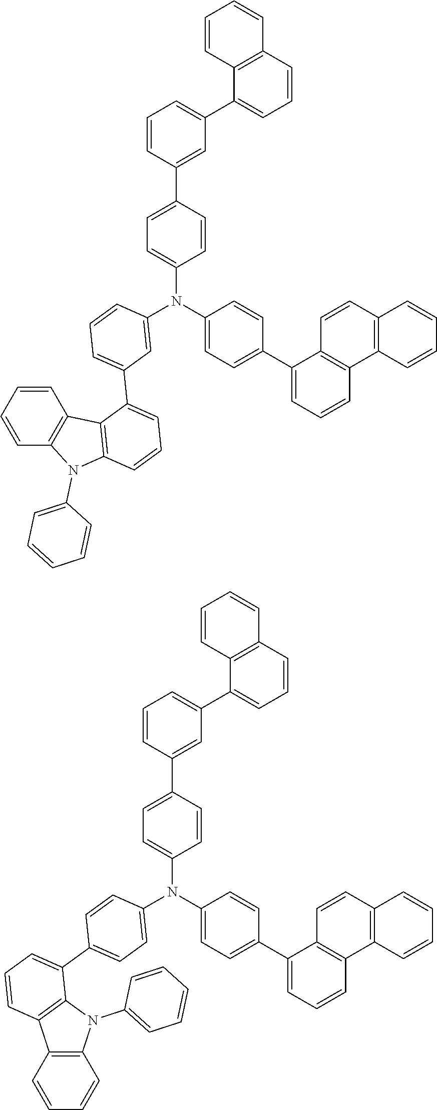 [SMILES: c1ccc(-n2c3ccccc3c3c(-c4cccc(N(c5ccc(-c6cccc(-c7cccc8ccccc78)c6)cc5)c5ccc(-c6cccc7c6ccc6ccccc67)cc5)c4)cccc32)cc1 ; c1ccc(-n2c3ccccc3c3cccc(-c4ccc(N(c5ccc(-c6cccc(-c7cccc8ccccc78)c6)cc5)c5ccc(-c6cccc7c6ccc6ccccc67)cc5)cc4)c32)cc1]

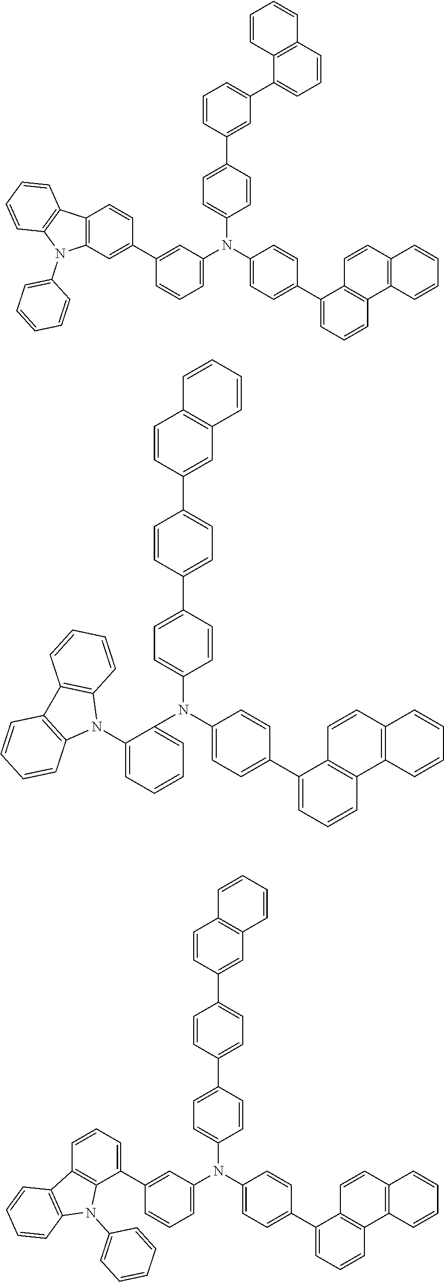 [SMILES: c1ccc(-n2c3ccccc3c3ccc(-c4cccc(N(c5ccc(-c6cccc(-c7cccc8ccccc78)c6)cc5)c5ccc(-c6cccc7c6ccc6ccccc67)cc5)c4)cc32)cc1 ; c1ccc(-n2c3ccccc3c3ccccc32)c(N(c2ccc(-c3ccc(-c4ccc5ccccc5c4)cc3)cc2)c2ccc(-c3cccc4c3ccc3ccccc34)cc2)c1 ; c1ccc(-n2c3ccccc3c3cccc(-c4cccc(N(c5ccc(-c6ccc(-c7ccc8ccccc8c7)cc6)cc5)c5ccc(-c6cccc7c6ccc6ccccc67)cc5)c4)c32)cc1]

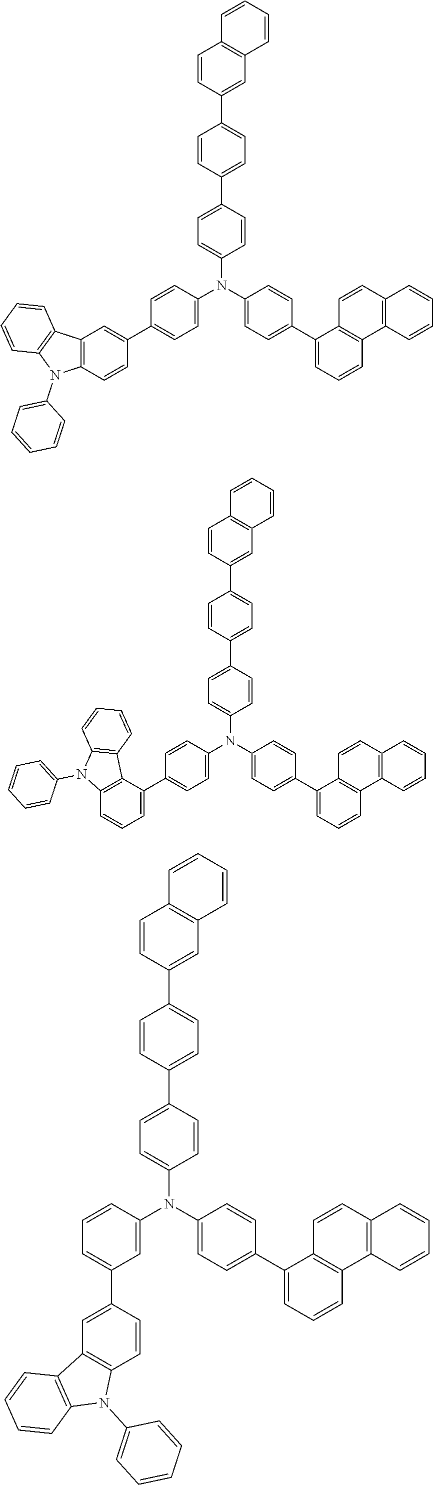 [SMILES: c1ccc(-n2c3ccccc3c3cc(-c4ccc(N(c5ccc(-c6ccc(-c7ccc8ccccc8c7)cc6)cc5)c5ccc(-c6cccc7c6ccc6ccccc67)cc5)cc4)ccc32)cc1 ; c1ccc(-n2c3ccccc3c3c(-c4ccc(N(c5ccc(-c6ccc(-c7ccc8ccccc8c7)cc6)cc5)c5ccc(-c6cccc7c6ccc6ccccc67)cc5)cc4)cccc32)cc1 ; c1ccc(-n2c3ccccc3c3cc(-c4cccc(N(c5ccc(-c6ccc(-c7ccc8ccccc8c7)cc6)cc5)c5ccc(-c6cccc7c6ccc6ccccc67)cc5)c4)ccc32)cc1]

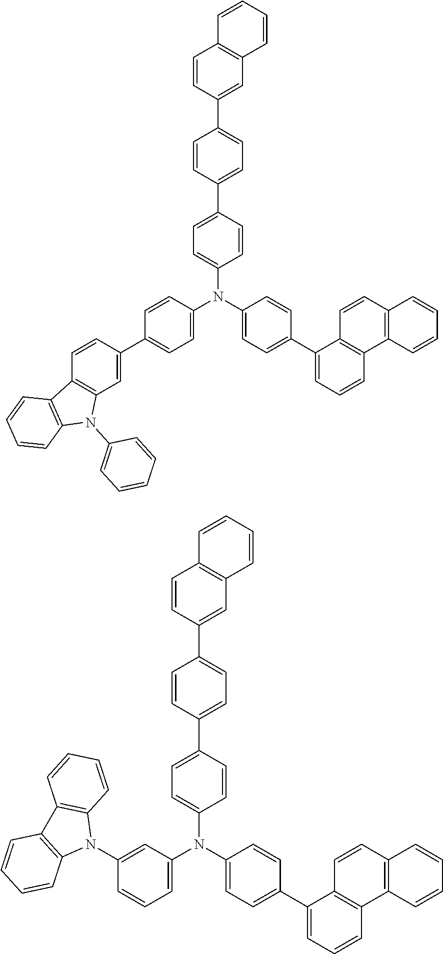 [SMILES: c1ccc(-n2c3ccccc3c3ccc(-c4ccc(N(c5ccc(-c6ccc(-c7ccc8ccccc8c7)cc6)cc5)c5ccc(-c6cccc7c6ccc6ccccc67)cc5)cc4)cc32)cc1 ; c1cc(N(c2ccc(-c3ccc(-c4ccc5ccccc5c4)cc3)cc2)c2ccc(-c3cccc4c3ccc3ccccc34)cc2)cc(-n2c3ccccc3c3ccccc32)c1]

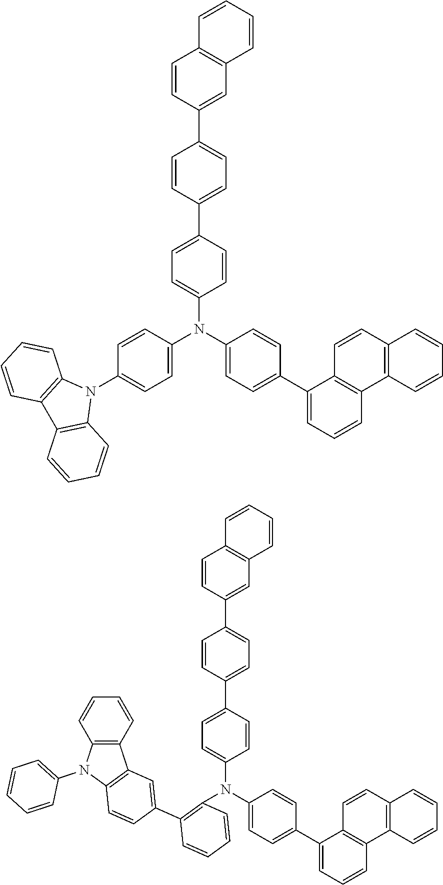 [SMILES: c1ccc2cc(-c3ccc(-c4ccc(N(c5ccc(-c6cccc7c6ccc6ccccc67)cc5)c5ccc(-n6c7ccccc7c7ccccc76)cc5)cc4)cc3)ccc2c1 ; c1ccc(-n2c3ccccc3c3cc(-c4ccccc4N(c4ccc(-c5ccc(-c6ccc7ccccc7c6)cc5)cc4)c4ccc(-c5cccc6c5ccc5ccccc56)cc4)ccc32)cc1]

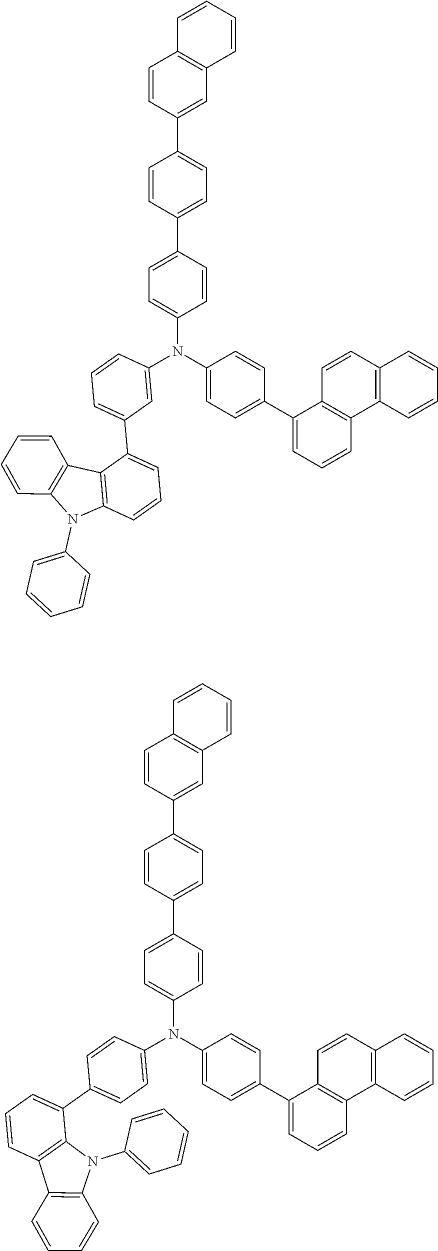 [SMILES: c1ccc(-n2c3ccccc3c3c(-c4cccc(N(c5ccc(-c6ccc(-c7ccc8ccccc8c7)cc6)cc5)c5ccc(-c6cccc7c6ccc6ccccc67)cc5)c4)cccc32)cc1 ; c1ccc(-n2c3ccccc3c3cccc(-c4ccc(N(c5ccc(-c6ccc(-c7ccc8ccccc8c7)cc6)cc5)c5ccc(-c6cccc7c6ccc6ccccc67)cc5)cc4)c32)cc1]

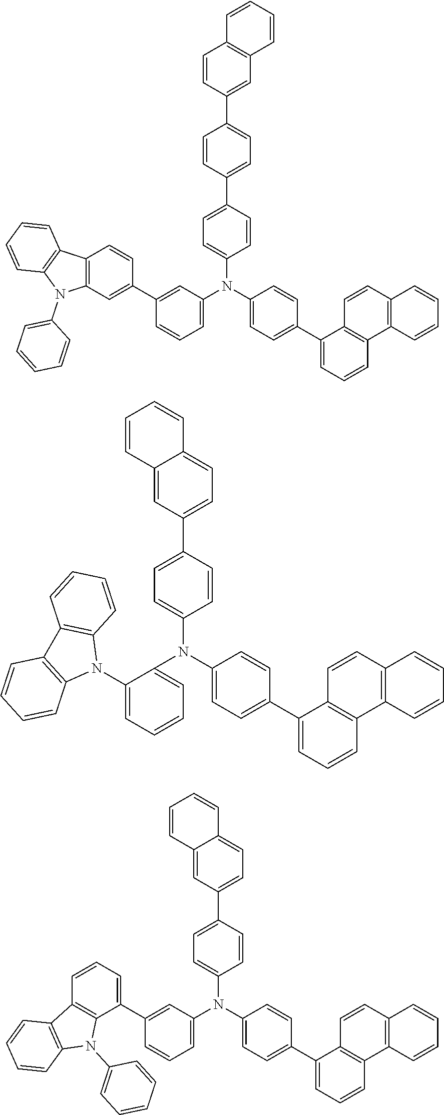 [SMILES: c1ccc(-n2c3ccccc3c3ccc(-c4cccc(N(c5ccc(-c6ccc(-c7ccc8ccccc8c7)cc6)cc5)c5ccc(-c6cccc7c6ccc6ccccc67)cc5)c4)cc32)cc1 ; c1ccc(-n2c3ccccc3c3ccccc32)c(N(c2ccc(-c3ccc4ccccc4c3)cc2)c2ccc(-c3cccc4c3ccc3ccccc34)cc2)c1 ; c1ccc(-n2c3ccccc3c3cccc(-c4cccc(N(c5ccc(-c6ccc7ccccc7c6)cc5)c5ccc(-c6cccc7c6ccc6ccccc67)cc5)c4)c32)cc1]

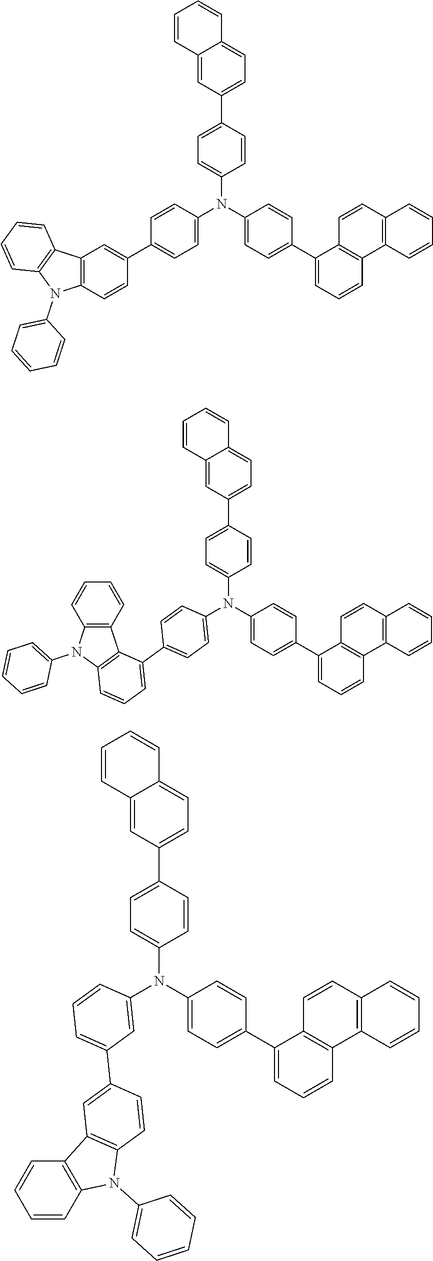

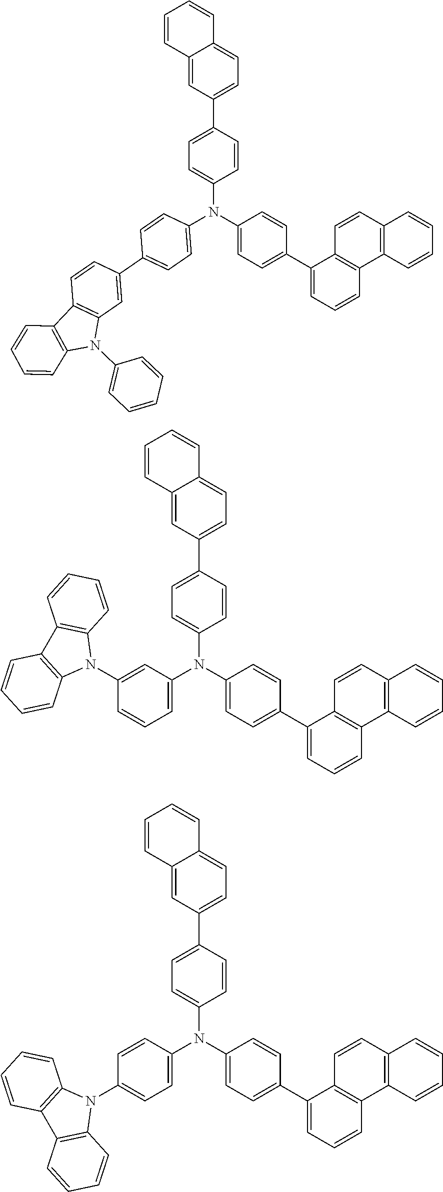

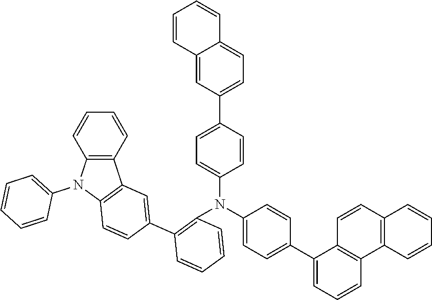 [SMILES: c1ccc(-n2c3ccccc3c3cc(-c4ccccc4N(c4ccc(-c5ccc6ccccc6c5)cc4)c4ccc(-c5cccc6c5ccc5ccccc56)cc4)ccc32)cc1]

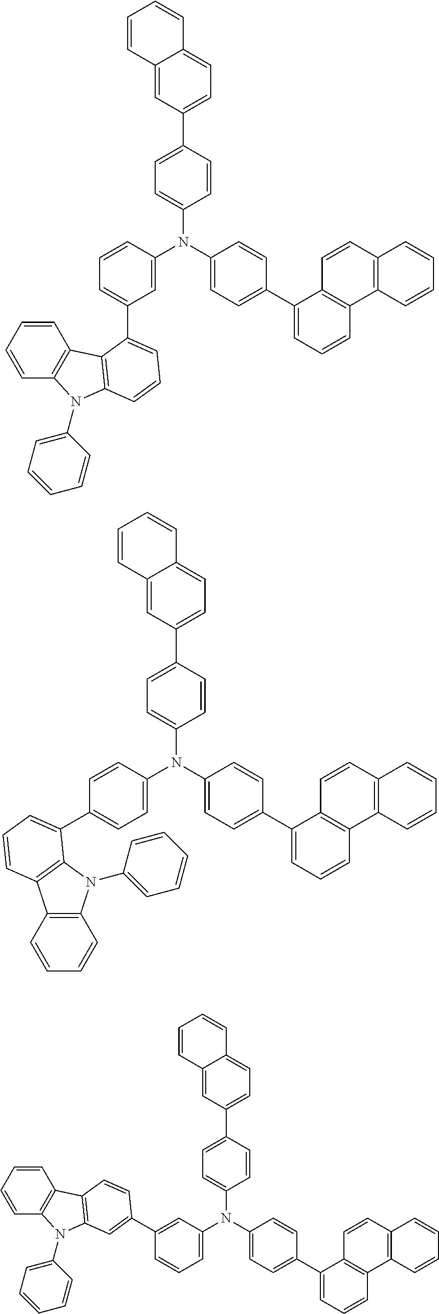 [SMILES: c1ccc(-n2c3ccccc3c3c(-c4cccc(N(c5ccc(-c6ccc7ccccc7c6)cc5)c5ccc(-c6cccc7c6ccc6ccccc67)cc5)c4)cccc32)cc1 ; c1ccc(-n2c3ccccc3c3cccc(-c4ccc(N(c5ccc(-c6ccc7ccccc7c6)cc5)c5ccc(-c6cccc7c6ccc6ccccc67)cc5)cc4)c32)cc1 ; c1ccc(-n2c3ccccc3c3ccc(-c4cccc(N(c5ccc(-c6ccc7ccccc7c6)cc5)c5ccc(-c6cccc7c6ccc6ccccc67)cc5)c4)cc32)cc1]

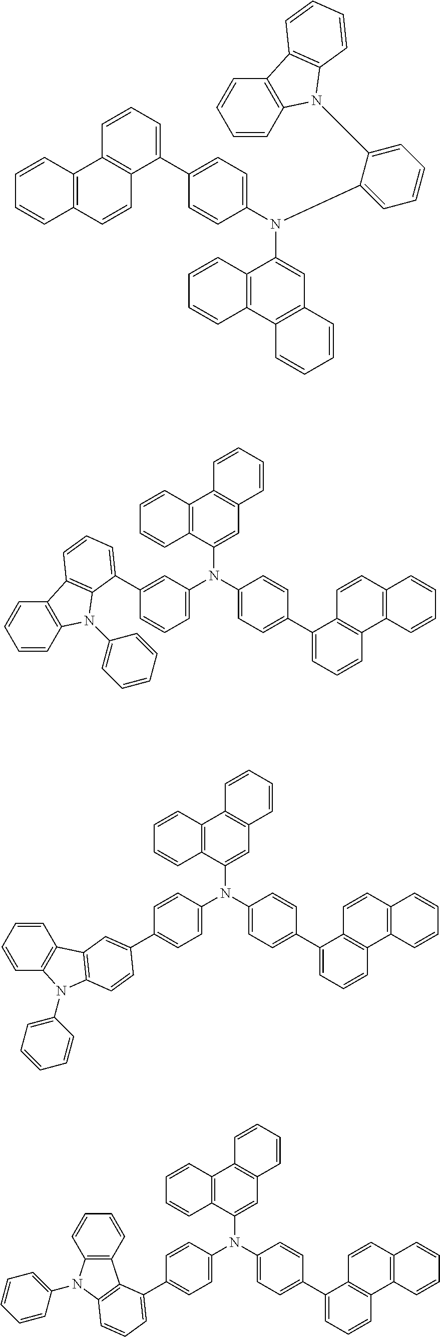 [SMILES: c1ccc(-n2c3ccccc3c3ccccc32)c(N(c2ccc(-c3cccc4c3ccc3ccccc34)cc2)c2cc3ccccc3c3ccccc23)c1 ; c1ccc(-n2c3ccccc3c3cccc(-c4cccc(N(c5ccc(-c6cccc7c6ccc6ccccc67)cc5)c5cc6ccccc6c6ccccc56)c4)c32)cc1 ; c1ccc(-n2c3ccccc3c3cc(-c4ccc(N(c5ccc(-c6cccc7c6ccc6ccccc67)cc5)c5cc6ccccc6c6ccccc56)cc4)ccc32)cc1 ; c1ccc(-n2c3ccccc3c3c(-c4ccc(N(c5ccc(-c6cccc7c6ccc6ccccc67)cc5)c5cc6ccccc6c6ccccc56)cc4)cccc32)cc1]

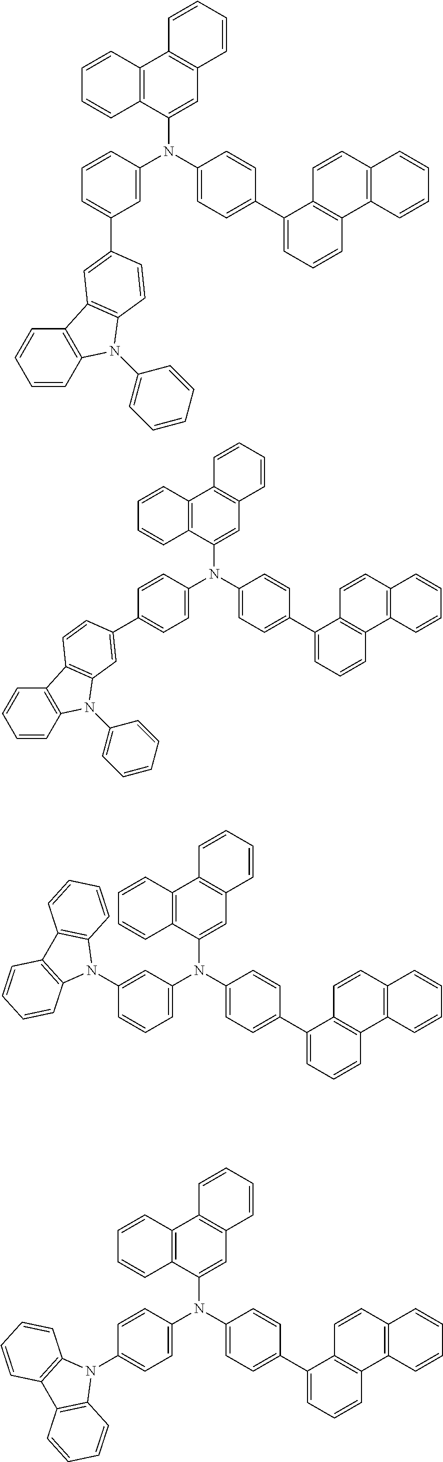 [SMILES: c1ccc(-n2c3ccccc3c3cc(-c4cccc(N(c5ccc(-c6cccc7c6ccc6ccccc67)cc5)c5cc6ccccc6c6ccccc56)c4)ccc32)cc1 ; c1ccc(-n2c3ccccc3c3ccc(-c4ccc(N(c5ccc(-c6cccc7c6ccc6ccccc67)cc5)c5cc6ccccc6c6ccccc56)cc4)cc32)cc1 ; c1cc(N(c2ccc(-c3cccc4c3ccc3ccccc34)cc2)c2cc3ccccc3c3ccccc23)cc(-n2c3ccccc3c3ccccc32)c1 ; c1ccc2c(c1)ccc1c(-c3ccc(N(c4ccc(-n5c6ccccc6c6ccccc65)cc4)c4cc5ccccc5c5ccccc45)cc3)cccc12]

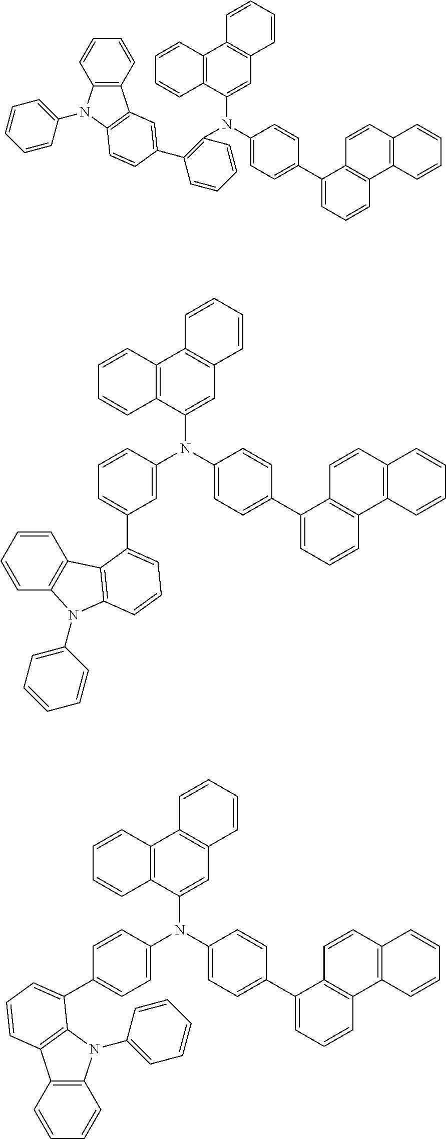 [SMILES: c1ccc(-n2c3ccccc3c3cc(-c4ccccc4N(c4ccc(-c5cccc6c5ccc5ccccc56)cc4)c4cc5ccccc5c5ccccc45)ccc32)cc1 ; c1ccc(-n2c3ccccc3c3c(-c4cccc(N(c5ccc(-c6cccc7c6ccc6ccccc67)cc5)c5cc6ccccc6c6ccccc56)c4)cccc32)cc1 ; c1ccc(-n2c3ccccc3c3cccc(-c4ccc(N(c5ccc(-c6cccc7c6ccc6ccccc67)cc5)c5cc6ccccc6c6ccccc56)cc4)c32)cc1]

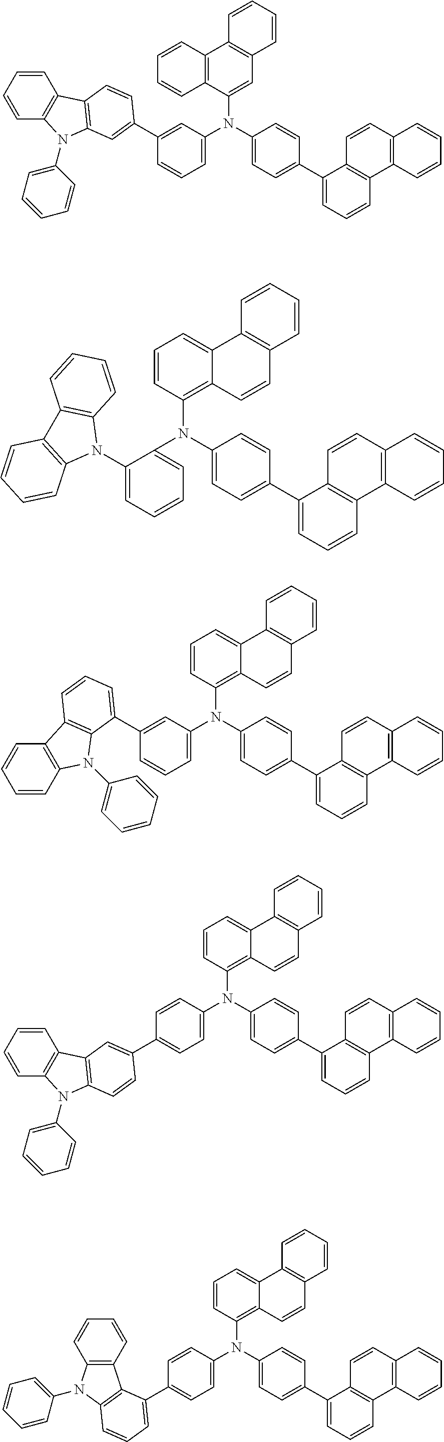 [SMILES: c1ccc(-n2c3ccccc3c3ccc(-c4cccc(N(c5ccc(-c6cccc7c6ccc6ccccc67)cc5)c5cc6ccccc6c6ccccc56)c4)cc32)cc1 ; c1ccc(-n2c3ccccc3c3ccccc32)c(N(c2ccc(-c3cccc4c3ccc3ccccc34)cc2)c2cccc3c2ccc2ccccc23)c1 ; c1ccc(-n2c3ccccc3c3cccc(-c4cccc(N(c5ccc(-c6cccc7c6ccc6ccccc67)cc5)c5cccc6c5ccc5ccccc56)c4)c32)cc1 ; c1ccc(-n2c3ccccc3c3cc(-c4ccc(N(c5ccc(-c6cccc7c6ccc6ccccc67)cc5)c5cccc6c5ccc5ccccc56)cc4)ccc32)cc1 ; c1ccc(-n2c3ccccc3c3c(-c4ccc(N(c5ccc(-c6cccc7c6ccc6ccccc67)cc5)c5cccc6c5ccc5ccccc56)cc4)cccc32)cc1]

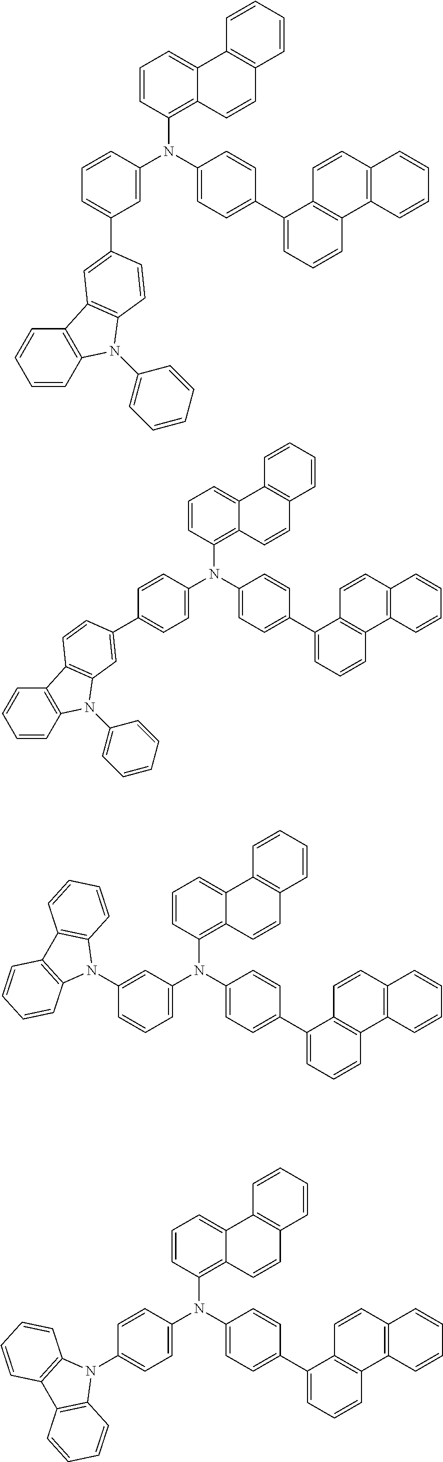 [SMILES: c1ccc(-n2c3ccccc3c3cc(-c4cccc(N(c5ccc(-c6cccc7c6ccc6ccccc67)cc5)c5cccc6c5ccc5ccccc56)c4)ccc32)cc1 ; c1ccc(-n2c3ccccc3c3ccc(-c4ccc(N(c5ccc(-c6cccc7c6ccc6ccccc67)cc5)c5cccc6c5ccc5ccccc56)cc4)cc32)cc1 ; c1cc(N(c2ccc(-c3cccc4c3ccc3ccccc34)cc2)c2cccc3c2ccc2ccccc23)cc(-n2c3ccccc3c3ccccc32)c1 ; c1ccc2c(c1)ccc1c(-c3ccc(N(c4ccc(-n5c6ccccc6c6ccccc65)cc4)c4cccc5c4ccc4ccccc45)cc3)cccc12]

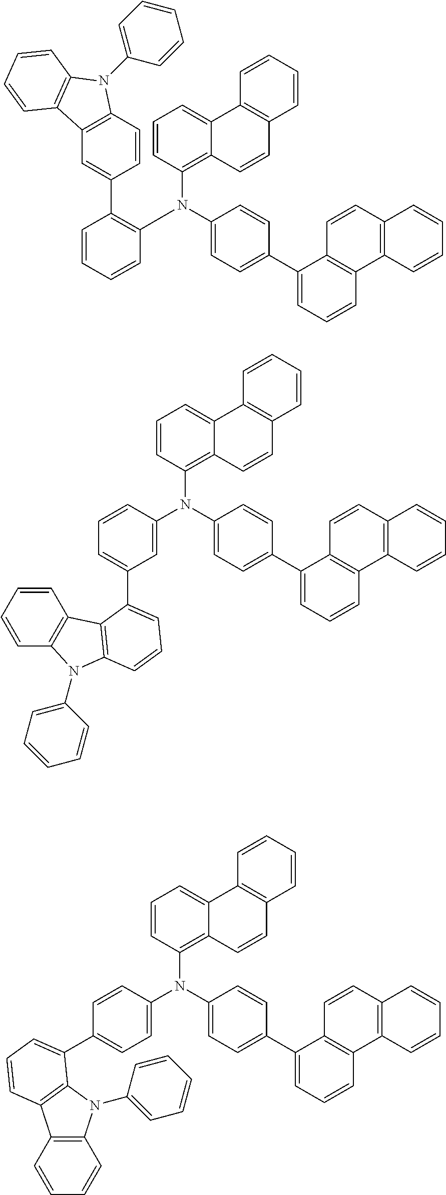 [SMILES: c1ccc(-n2c3ccccc3c3cc(-c4ccccc4N(c4ccc(-c5cccc6c5ccc5ccccc56)cc4)c4cccc5c4ccc4ccccc45)ccc32)cc1 ; c1ccc(-n2c3ccccc3c3c(-c4cccc(N(c5ccc(-c6cccc7c6ccc6ccccc67)cc5)c5cccc6c5ccc5ccccc56)c4)cccc32)cc1 ; c1ccc(-n2c3ccccc3c3cccc(-c4ccc(N(c5ccc(-c6cccc7c6ccc6ccccc67)cc5)c5cccc6c5ccc5ccccc56)cc4)c32)cc1]

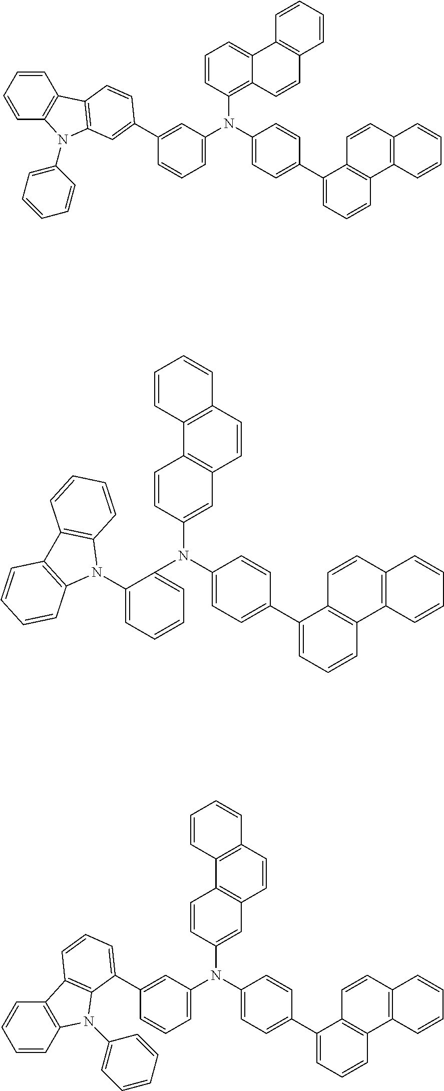 [SMILES: c1ccc(-n2c3ccccc3c3ccc(-c4cccc(N(c5ccc(-c6cccc7c6ccc6ccccc67)cc5)c5cccc6c5ccc5ccccc56)c4)cc32)cc1 ; c1ccc(-n2c3ccccc3c3ccccc32)c(N(c2ccc(-c3cccc4c3ccc3ccccc34)cc2)c2ccc3c(ccc4ccccc43)c2)c1 ; c1ccc(-n2c3ccccc3c3cccc(-c4cccc(N(c5ccc(-c6cccc7c6ccc6ccccc67)cc5)c5ccc6c(ccc7ccccc76)c5)c4)c32)cc1]

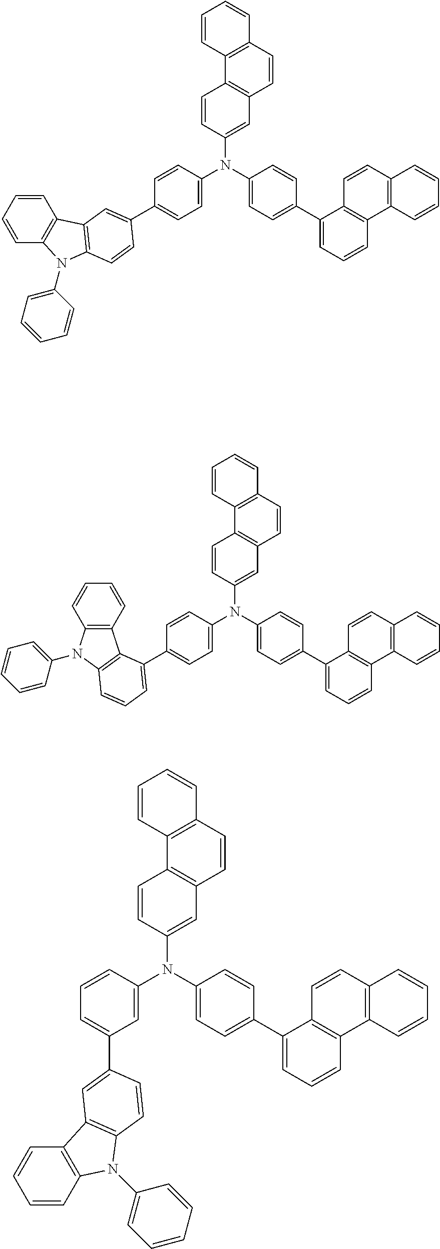 [SMILES: c1ccc(-n2c3ccccc3c3cc(-c4ccc(N(c5ccc(-c6cccc7c6ccc6ccccc67)cc5)c5ccc6c(ccc7ccccc76)c5)cc4)ccc32)cc1 ; c1ccc(-n2c3ccccc3c3c(-c4ccc(N(c5ccc(-c6cccc7c6ccc6ccccc67)cc5)c5ccc6c(ccc7ccccc76)c5)cc4)cccc32)cc1 ; c1ccc(-n2c3ccccc3c3cc(-c4cccc(N(c5ccc(-c6cccc7c6ccc6ccccc67)cc5)c5ccc6c(ccc7ccccc76)c5)c4)ccc32)cc1]

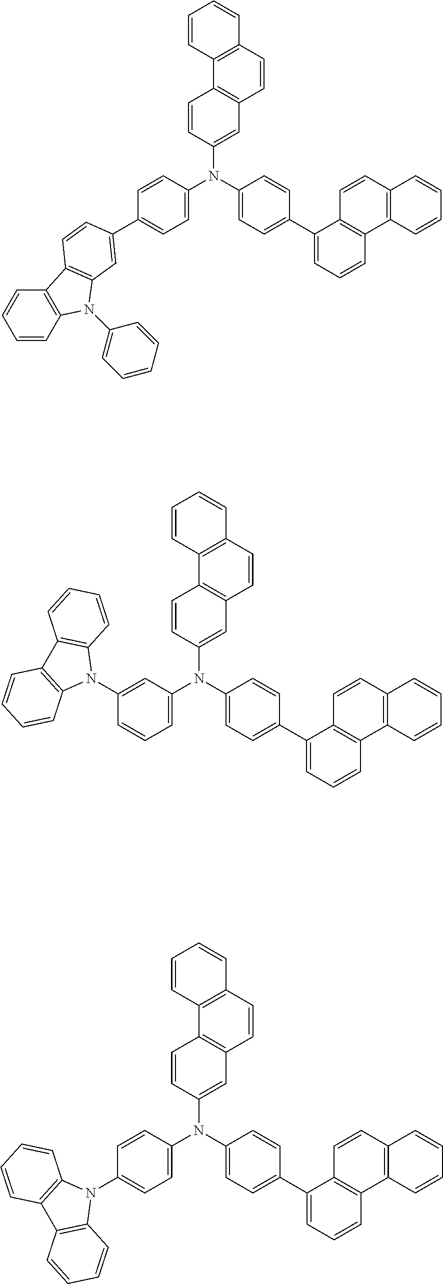 [SMILES: c1ccc(-n2c3ccccc3c3ccc(-c4ccc(N(c5ccc(-c6cccc7c6ccc6ccccc67)cc5)c5ccc6c(ccc7ccccc76)c5)cc4)cc32)cc1 ; c1cc(N(c2ccc(-c3cccc4c3ccc3ccccc34)cc2)c2ccc3c(ccc4ccccc43)c2)cc(-n2c3ccccc3c3ccccc32)c1 ; c1ccc2c(c1)ccc1cc(N(c3ccc(-c4cccc5c4ccc4ccccc45)cc3)c3ccc(-n4c5ccccc5c5ccccc54)cc3)ccc12]

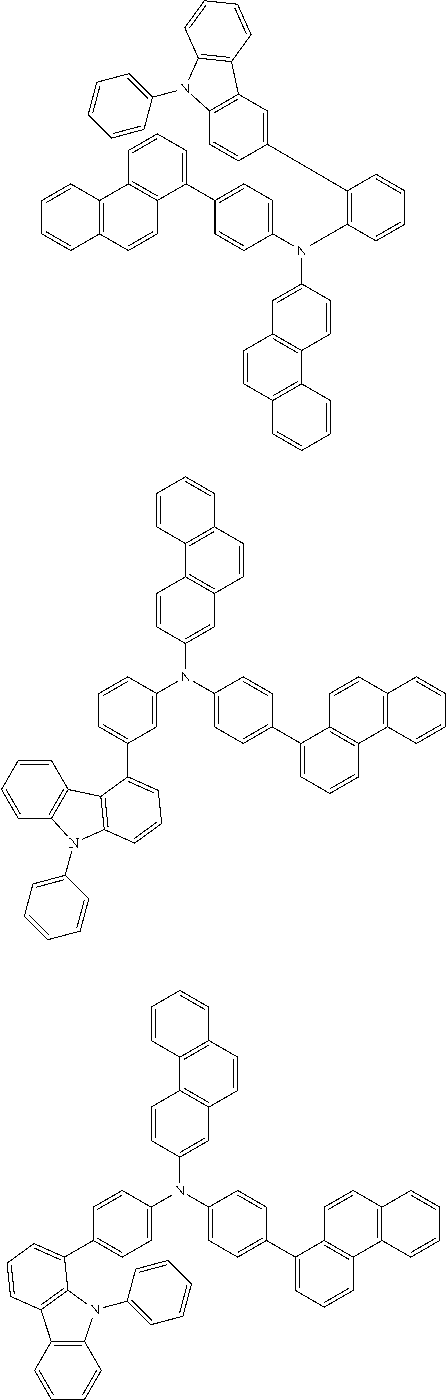 [SMILES: c1ccc(-n2c3ccccc3c3cc(-c4ccccc4N(c4ccc(-c5cccc6c5ccc5ccccc56)cc4)c4ccc5c(ccc6ccccc65)c4)ccc32)cc1 ; c1ccc(-n2c3ccccc3c3c(-c4cccc(N(c5ccc(-c6cccc7c6ccc6ccccc67)cc5)c5ccc6c(ccc7ccccc76)c5)c4)cccc32)cc1 ; c1ccc(-n2c3ccccc3c3cccc(-c4ccc(N(c5ccc(-c6cccc7c6ccc6ccccc67)cc5)c5ccc6c(ccc7ccccc76)c5)cc4)c32)cc1]

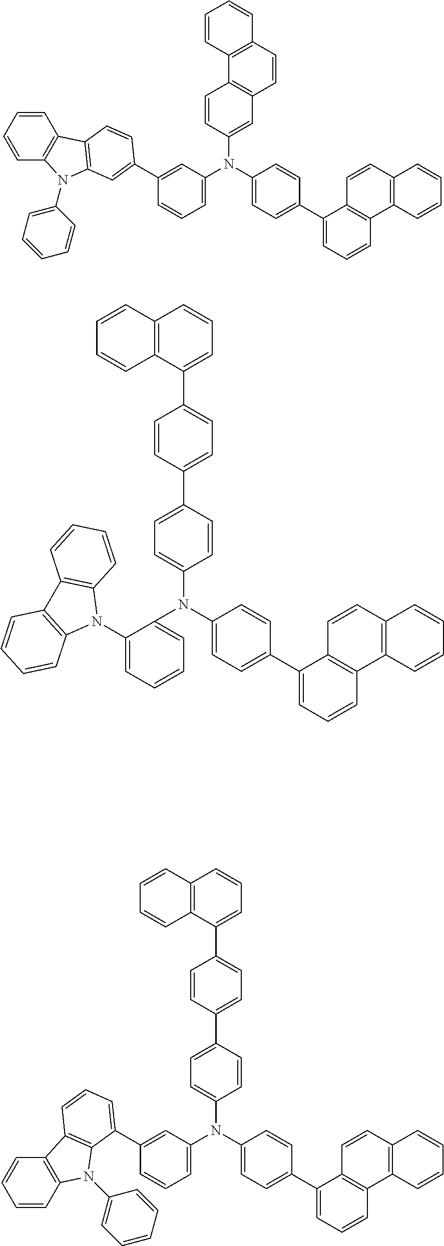 [SMILES: c1ccc(-n2c3ccccc3c3ccc(-c4cccc(N(c5ccc(-c6cccc7c6ccc6ccccc67)cc5)c5ccc6c(ccc7ccccc76)c5)c4)cc32)cc1 ; c1ccc(-n2c3ccccc3c3ccccc32)c(N(c2ccc(-c3ccc(-c4cccc5ccccc45)cc3)cc2)c2ccc(-c3cccc4c3ccc3ccccc34)cc2)c1 ; c1ccc(-n2c3ccccc3c3cccc(-c4cccc(N(c5ccc(-c6ccc(-c7cccc8ccccc78)cc6)cc5)c5ccc(-c6cccc7c6ccc6ccccc67)cc5)c4)c32)cc1]

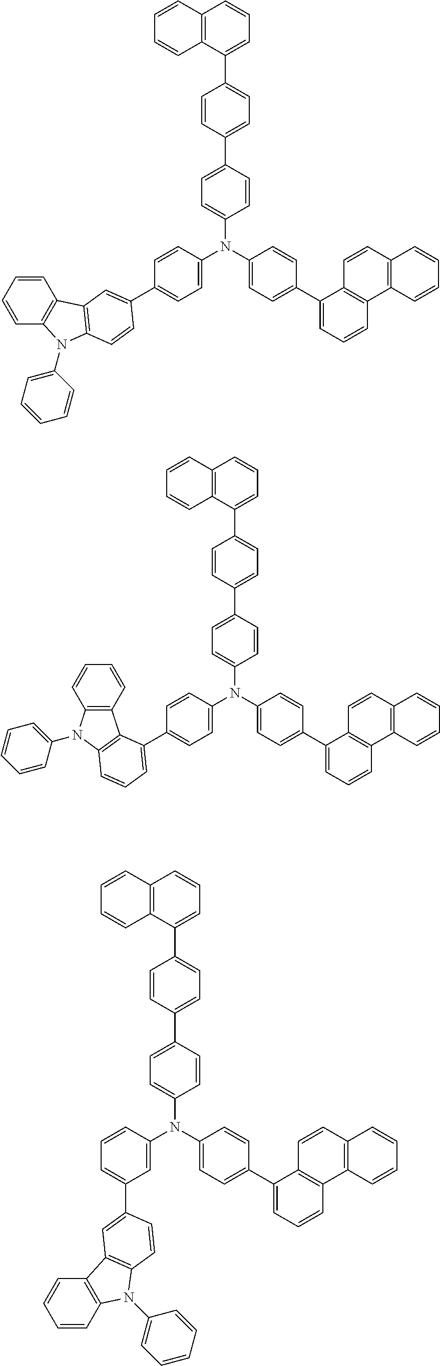 [SMILES: c1ccc(-n2c3ccccc3c3cc(-c4ccc(N(c5ccc(-c6ccc(-c7cccc8ccccc78)cc6)cc5)c5ccc(-c6cccc7c6ccc6ccccc67)cc5)cc4)ccc32)cc1 ; c1ccc(-n2c3ccccc3c3c(-c4ccc(N(c5ccc(-c6ccc(-c7cccc8ccccc78)cc6)cc5)c5ccc(-c6cccc7c6ccc6ccccc67)cc5)cc4)cccc32)cc1 ; c1ccc(-n2c3ccccc3c3cc(-c4cccc(N(c5ccc(-c6ccc(-c7cccc8ccccc78)cc6)cc5)c5ccc(-c6cccc7c6ccc6ccccc67)cc5)c4)ccc32)cc1]

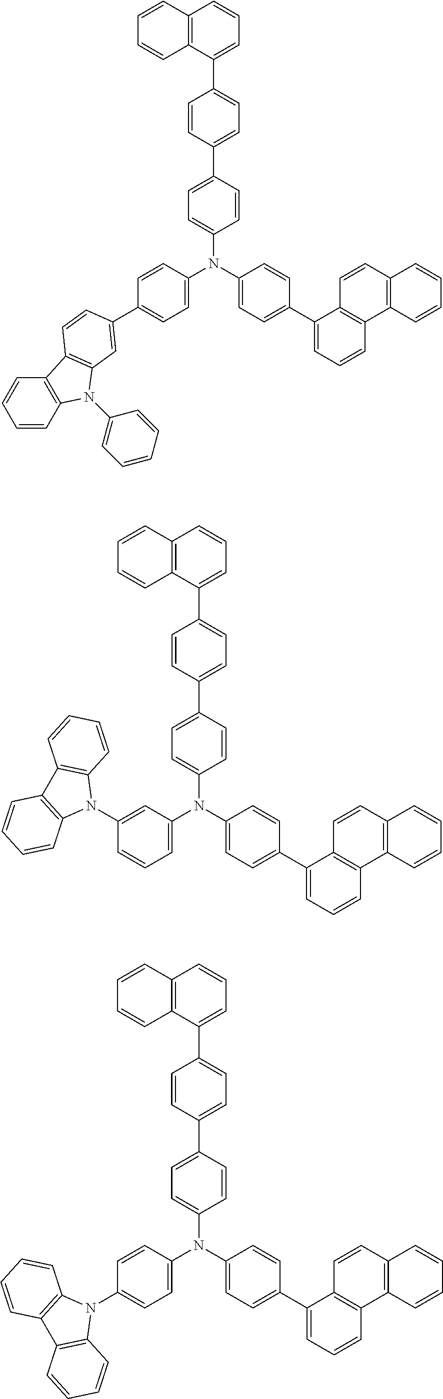 [SMILES: c1ccc(-n2c3ccccc3c3ccc(-c4ccc(N(c5ccc(-c6ccc(-c7cccc8ccccc78)cc6)cc5)c5ccc(-c6cccc7c6ccc6ccccc67)cc5)cc4)cc32)cc1 ; c1cc(N(c2ccc(-c3ccc(-c4cccc5ccccc45)cc3)cc2)c2ccc(-c3cccc4c3ccc3ccccc34)cc2)cc(-n2c3ccccc3c3ccccc32)c1 ; c1ccc2c(-c3ccc(-c4ccc(N(c5ccc(-c6cccc7c6ccc6ccccc67)cc5)c5ccc(-n6c7ccccc7c7ccccc76)cc5)cc4)cc3)cccc2c1]

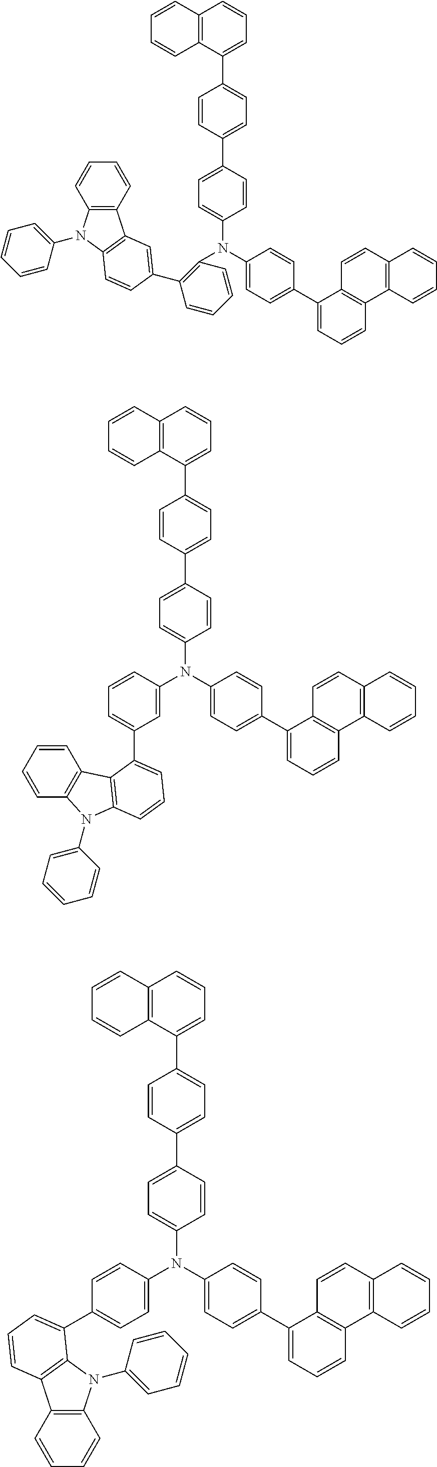 [SMILES: c1ccc(-n2c3ccccc3c3cc(-c4ccccc4N(c4ccc(-c5ccc(-c6cccc7ccccc67)cc5)cc4)c4ccc(-c5cccc6c5ccc5ccccc56)cc4)ccc32)cc1 ; c1ccc(-n2c3ccccc3c3c(-c4cccc(N(c5ccc(-c6ccc(-c7cccc8ccccc78)cc6)cc5)c5ccc(-c6cccc7c6ccc6ccccc67)cc5)c4)cccc32)cc1 ; c1ccc(-n2c3ccccc3c3cccc(-c4ccc(N(c5ccc(-c6ccc(-c7cccc8ccccc78)cc6)cc5)c5ccc(-c6cccc7c6ccc6ccccc67)cc5)cc4)c32)cc1]

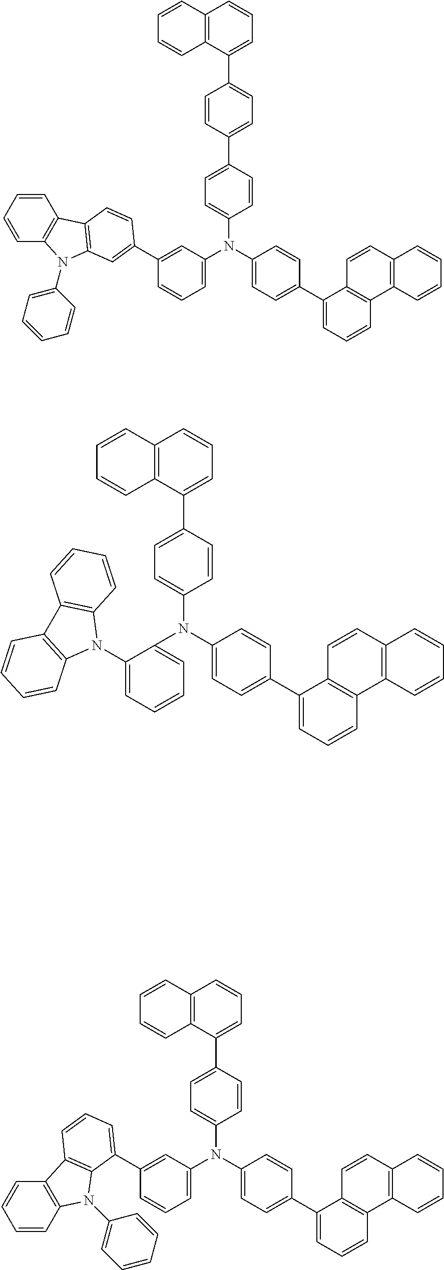 [SMILES: c1ccc(-n2c3ccccc3c3ccc(-c4cccc(N(c5ccc(-c6ccc(-c7cccc8ccccc78)cc6)cc5)c5ccc(-c6cccc7c6ccc6ccccc67)cc5)c4)cc32)cc1 ; c1ccc(-n2c3ccccc3c3ccccc32)c(N(c2ccc(-c3cccc4ccccc34)cc2)c2ccc(-c3cccc4c3ccc3ccccc34)cc2)c1 ; c1ccc(-n2c3ccccc3c3cccc(-c4cccc(N(c5ccc(-c6cccc7ccccc67)cc5)c5ccc(-c6cccc7c6ccc6ccccc67)cc5)c4)c32)cc1]

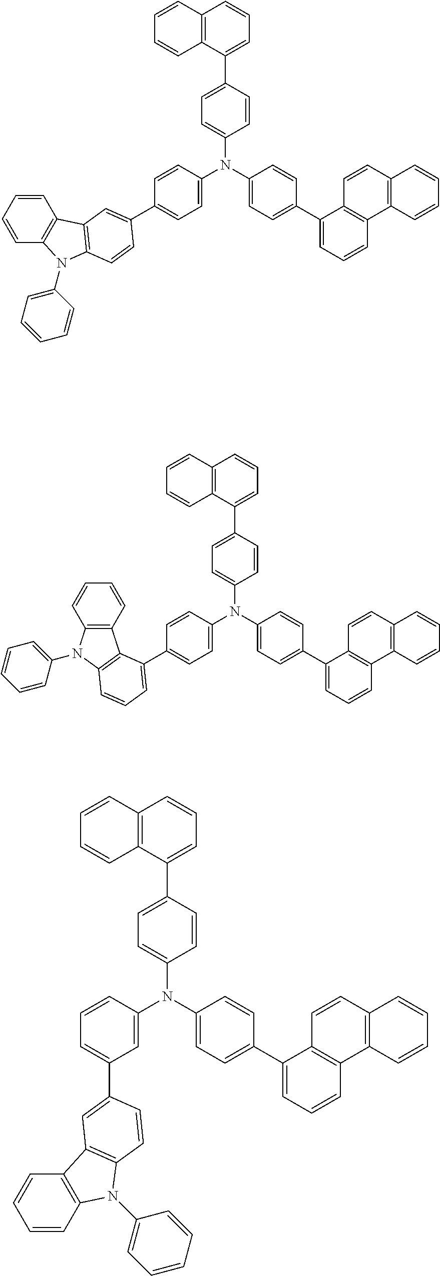 [SMILES: c1ccc(-n2c3ccccc3c3cc(-c4ccc(N(c5ccc(-c6cccc7ccccc67)cc5)c5ccc(-c6cccc7c6ccc6ccccc67)cc5)cc4)ccc32)cc1 ; c1ccc(-n2c3ccccc3c3c(-c4ccc(N(c5ccc(-c6cccc7ccccc67)cc5)c5ccc(-c6cccc7c6ccc6ccccc67)cc5)cc4)cccc32)cc1 ; c1ccc(-n2c3ccccc3c3cc(-c4cccc(N(c5ccc(-c6cccc7ccccc67)cc5)c5ccc(-c6cccc7c6ccc6ccccc67)cc5)c4)ccc32)cc1]

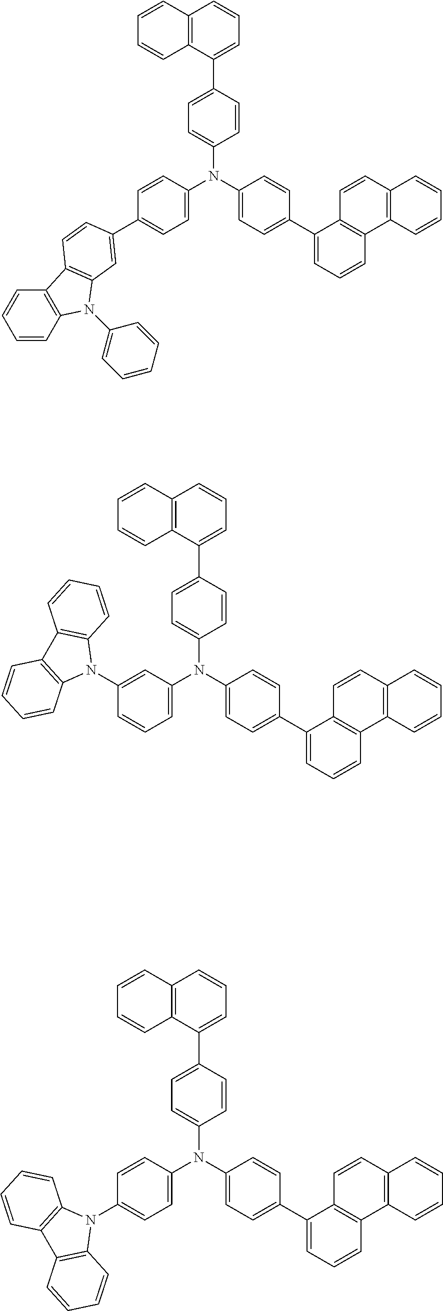 [SMILES: c1ccc(-n2c3ccccc3c3ccc(-c4ccc(N(c5ccc(-c6cccc7ccccc67)cc5)c5ccc(-c6cccc7c6ccc6ccccc67)cc5)cc4)cc32)cc1 ; c1cc(N(c2ccc(-c3cccc4ccccc34)cc2)c2ccc(-c3cccc4c3ccc3ccccc34)cc2)cc(-n2c3ccccc3c3ccccc32)c1 ; c1ccc2c(-c3ccc(N(c4ccc(-c5cccc6c5ccc5ccccc56)cc4)c4ccc(-n5c6ccccc6c6ccccc65)cc4)cc3)cccc2c1]

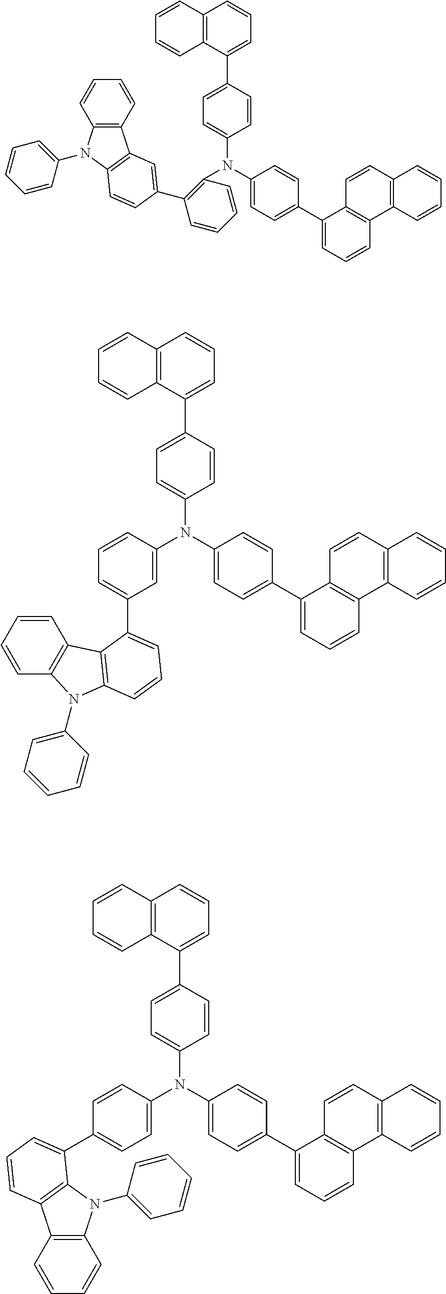 [SMILES: c1ccc(-n2c3ccccc3c3cc(-c4ccccc4N(c4ccc(-c5cccc6ccccc56)cc4)c4ccc(-c5cccc6c5ccc5ccccc56)cc4)ccc32)cc1 ; c1ccc(-n2c3ccccc3c3c(-c4cccc(N(c5ccc(-c6cccc7ccccc67)cc5)c5ccc(-c6cccc7c6ccc6ccccc67)cc5)c4)cccc32)cc1 ; c1ccc(-n2c3ccccc3c3cccc(-c4ccc(N(c5ccc(-c6cccc7ccccc67)cc5)c5ccc(-c6cccc7c6ccc6ccccc67)cc5)cc4)c32)cc1]

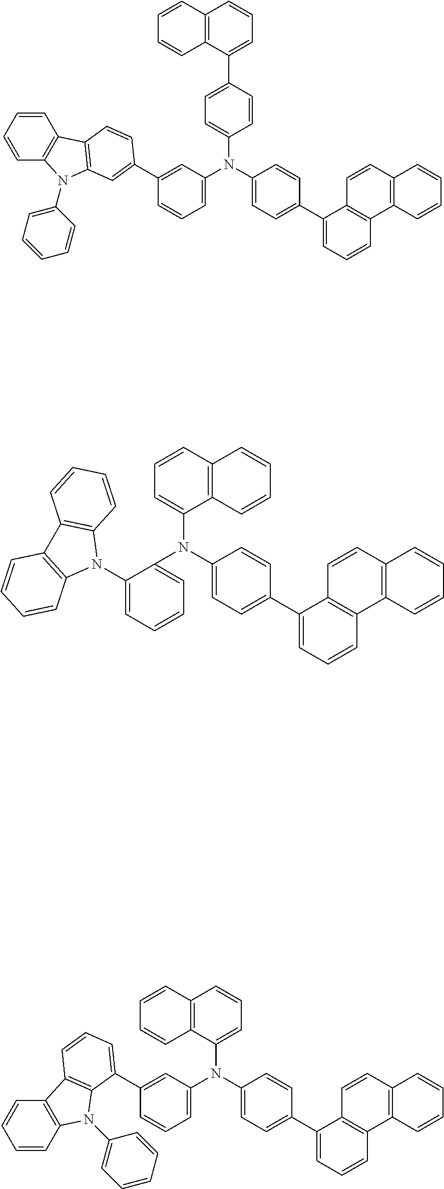 [SMILES: c1ccc(-n2c3ccccc3c3ccc(-c4cccc(N(c5ccc(-c6cccc7ccccc67)cc5)c5ccc(-c6cccc7c6ccc6ccccc67)cc5)c4)cc32)cc1 ; c1ccc(-n2c3ccccc3c3ccccc32)c(N(c2ccc(-c3cccc4c3ccc3ccccc34)cc2)c2cccc3ccccc23)c1 ; c1ccc(-n2c3ccccc3c3cccc(-c4cccc(N(c5ccc(-c6cccc7c6ccc6ccccc67)cc5)c5cccc6ccccc56)c4)c32)cc1]

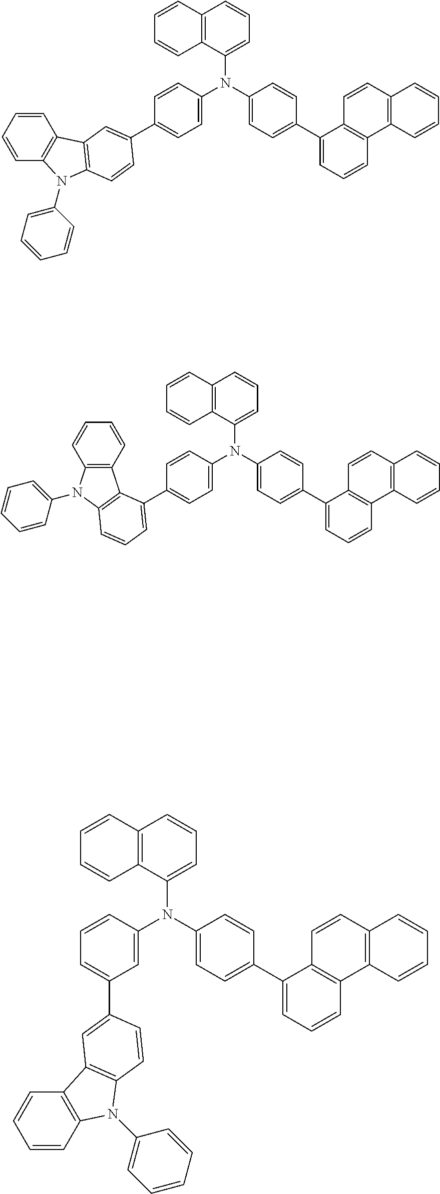 [SMILES: c1ccc(-n2c3ccccc3c3cc(-c4ccc(N(c5ccc(-c6cccc7c6ccc6ccccc67)cc5)c5cccc6ccccc56)cc4)ccc32)cc1 ; c1ccc(-n2c3ccccc3c3c(-c4ccc(N(c5ccc(-c6cccc7c6ccc6ccccc67)cc5)c5cccc6ccccc56)cc4)cccc32)cc1 ; c1ccc(-n2c3ccccc3c3cc(-c4cccc(N(c5ccc(-c6cccc7c6ccc6ccccc67)cc5)c5cccc6ccccc56)c4)ccc32)cc1]

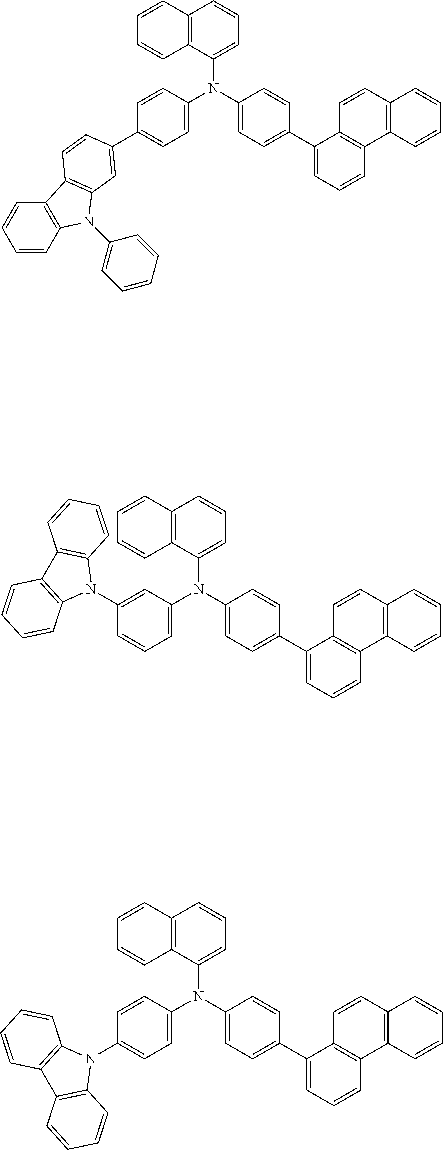 [SMILES: c1ccc(-n2c3ccccc3c3ccc(-c4ccc(N(c5ccc(-c6cccc7c6ccc6ccccc67)cc5)c5cccc6ccccc56)cc4)cc32)cc1 ; c1cc(N(c2ccc(-c3cccc4c3ccc3ccccc34)cc2)c2cccc3ccccc23)cc(-n2c3ccccc3c3ccccc32)c1 ; c1ccc2c(N(c3ccc(-c4cccc5c4ccc4ccccc45)cc3)c3ccc(-n4c5ccccc5c5ccccc54)cc3)cccc2c1]

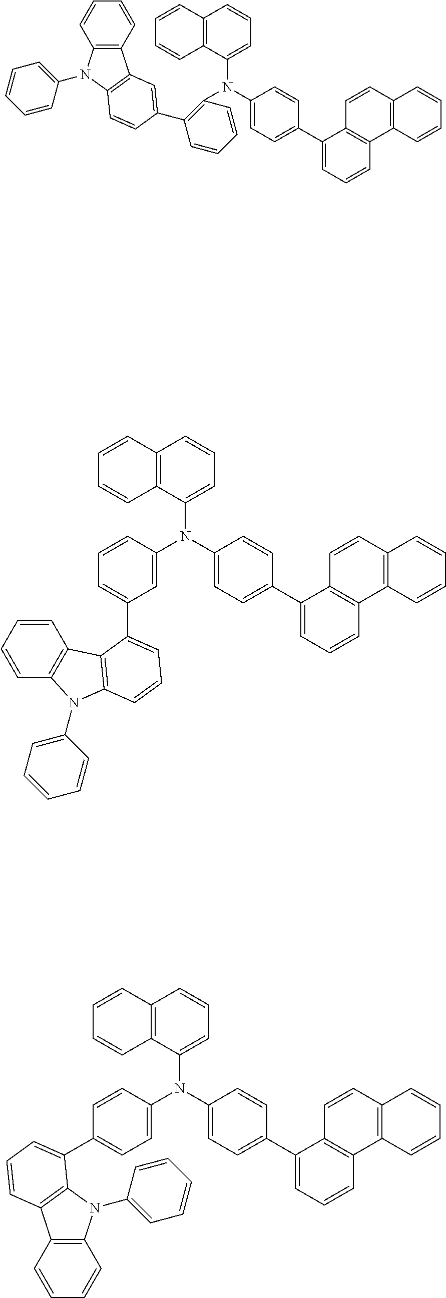 [SMILES: c1ccc(-n2c3ccccc3c3cc(-c4ccccc4N(c4ccc(-c5cccc6c5ccc5ccccc56)cc4)c4cccc5ccccc45)ccc32)cc1 ; c1ccc(-n2c3ccccc3c3c(-c4cccc(N(c5ccc(-c6cccc7c6ccc6ccccc67)cc5)c5cccc6ccccc56)c4)cccc32)cc1 ; c1ccc(-n2c3ccccc3c3cccc(-c4ccc(N(c5ccc(-c6cccc7c6ccc6ccccc67)cc5)c5cccc6ccccc56)cc4)c32)cc1]

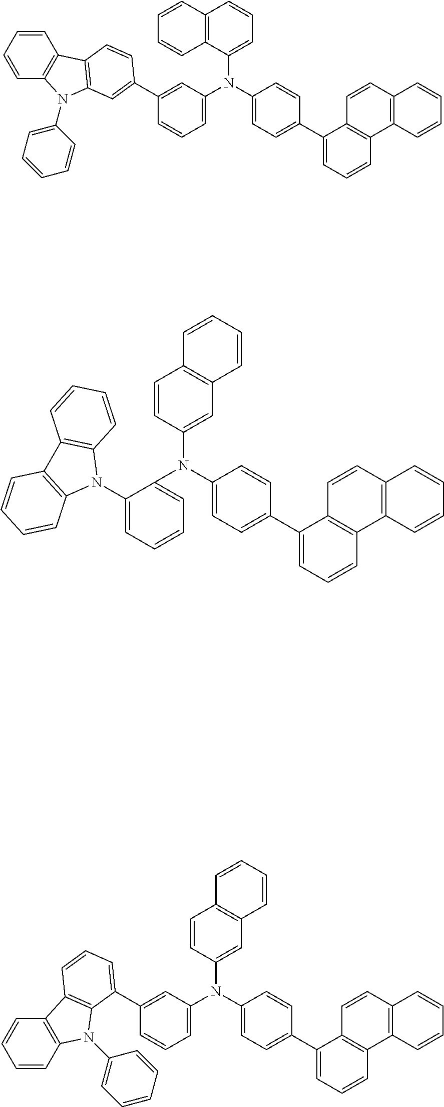 [SMILES: c1ccc(-n2c3ccccc3c3ccc(-c4cccc(N(c5ccc(-c6cccc7c6ccc6ccccc67)cc5)c5cccc6ccccc56)c4)cc32)cc1 ; c1ccc(-n2c3ccccc3c3ccccc32)c(N(c2ccc(-c3cccc4c3ccc3ccccc34)cc2)c2ccc3ccccc3c2)c1 ; c1ccc(-n2c3ccccc3c3cccc(-c4cccc(N(c5ccc(-c6cccc7c6ccc6ccccc67)cc5)c5ccc6ccccc6c5)c4)c32)cc1]

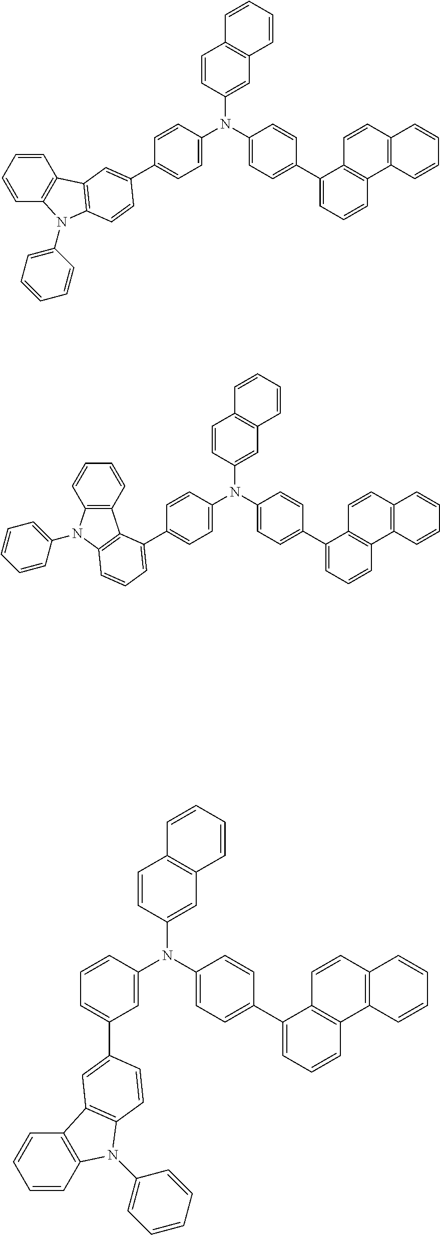 [SMILES: c1ccc(-n2c3ccccc3c3cc(-c4ccc(N(c5ccc(-c6cccc7c6ccc6ccccc67)cc5)c5ccc6ccccc6c5)cc4)ccc32)cc1 ; c1ccc(-n2c3ccccc3c3c(-c4ccc(N(c5ccc(-c6cccc7c6ccc6ccccc67)cc5)c5ccc6ccccc6c5)cc4)cccc32)cc1 ; c1ccc(-n2c3ccccc3c3cc(-c4cccc(N(c5ccc(-c6cccc7c6ccc6ccccc67)cc5)c5ccc6ccccc6c5)c4)ccc32)cc1]

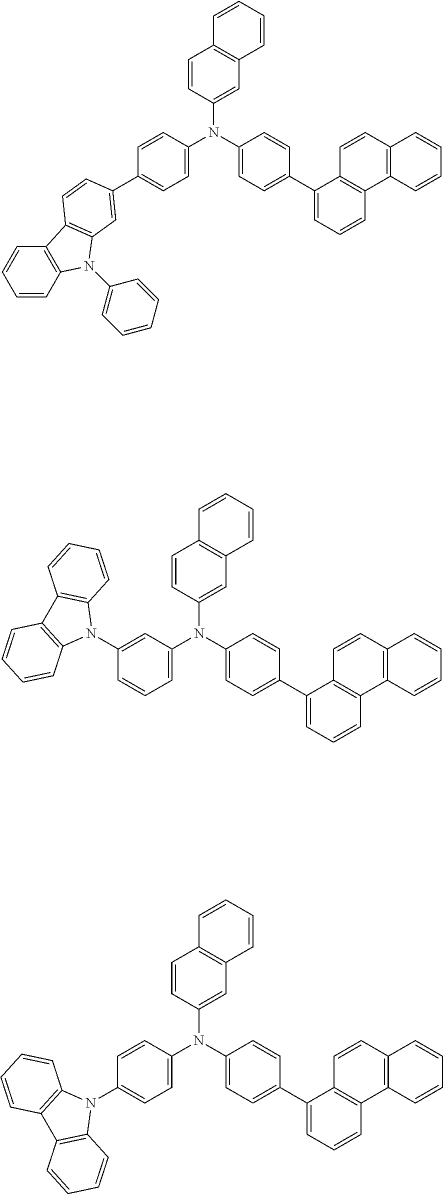 [SMILES: c1ccc(-n2c3ccccc3c3ccc(-c4ccc(N(c5ccc(-c6cccc7c6ccc6ccccc67)cc5)c5ccc6ccccc6c5)cc4)cc32)cc1 ; c1cc(N(c2ccc(-c3cccc4c3ccc3ccccc34)cc2)c2ccc3ccccc3c2)cc(-n2c3ccccc3c3ccccc32)c1 ; c1ccc2cc(N(c3ccc(-c4cccc5c4ccc4ccccc45)cc3)c3ccc(-n4c5ccccc5c5ccccc54)cc3)ccc2c1]

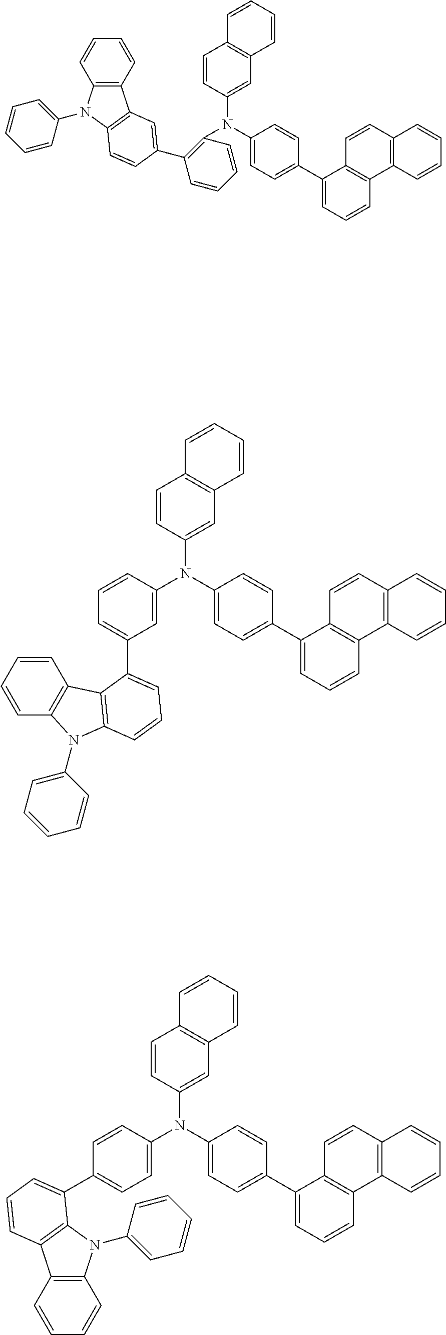 [SMILES: c1ccc(-n2c3ccccc3c3cc(-c4ccccc4N(c4ccc(-c5cccc6c5ccc5ccccc56)cc4)c4ccc5ccccc5c4)ccc32)cc1 ; c1ccc(-n2c3ccccc3c3c(-c4cccc(N(c5ccc(-c6cccc7c6ccc6ccccc67)cc5)c5ccc6ccccc6c5)c4)cccc32)cc1 ; c1ccc(-n2c3ccccc3c3cccc(-c4ccc(N(c5ccc(-c6cccc7c6ccc6ccccc67)cc5)c5ccc6ccccc6c5)cc4)c32)cc1]

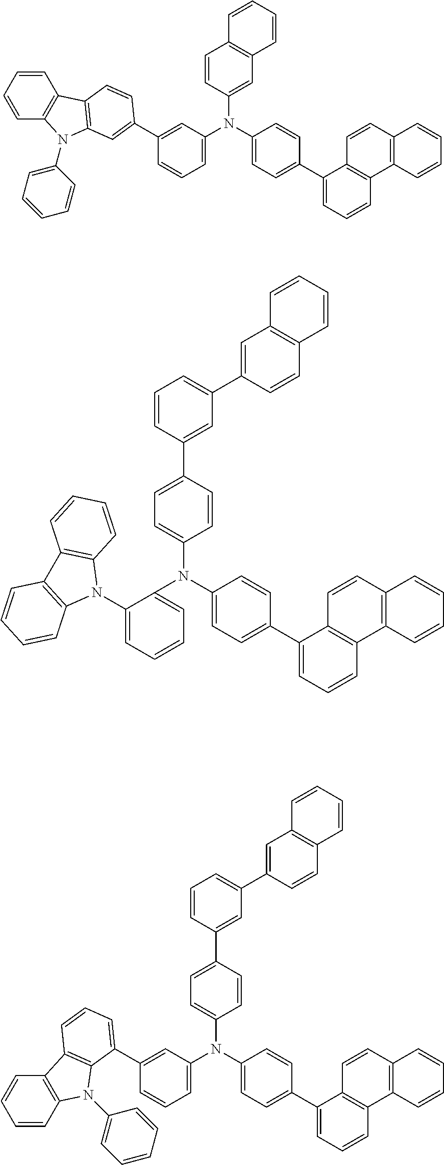 [SMILES: c1ccc(-n2c3ccccc3c3ccc(-c4cccc(N(c5ccc(-c6cccc7c6ccc6ccccc67)cc5)c5ccc6ccccc6c5)c4)cc32)cc1 ; c1cc(-c2ccc(N(c3ccc(-c4cccc5c4ccc4ccccc45)cc3)c3ccccc3-n3c4ccccc4c4ccccc43)cc2)cc(-c2ccc3ccccc3c2)c1 ; c1ccc(-n2c3ccccc3c3cccc(-c4cccc(N(c5ccc(-c6cccc(-c7ccc8ccccc8c7)c6)cc5)c5ccc(-c6cccc7c6ccc6ccccc67)cc5)c4)c32)cc1]

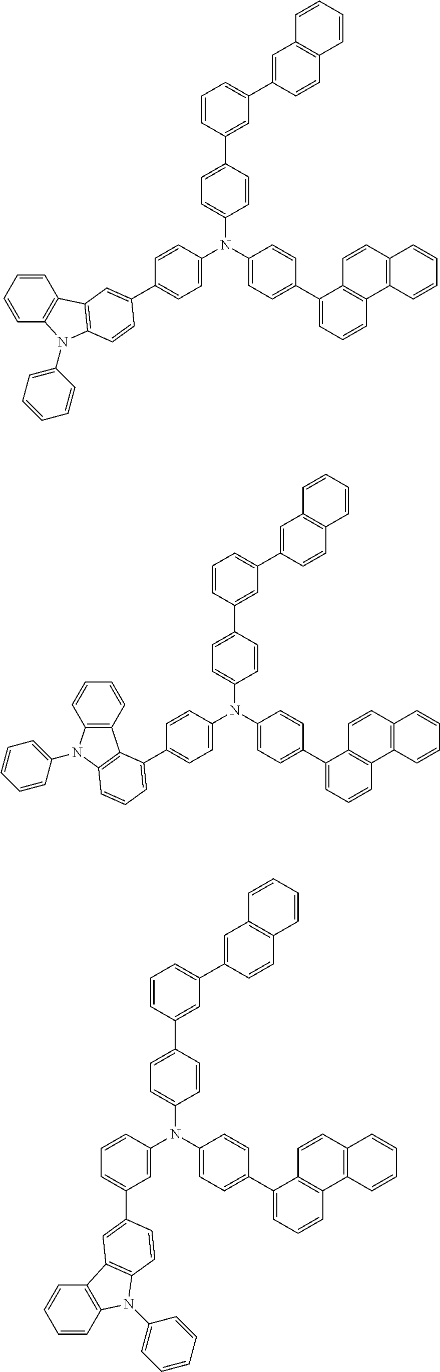 [SMILES: c1ccc(-n2c3ccccc3c3cc(-c4ccc(N(c5ccc(-c6cccc(-c7ccc8ccccc8c7)c6)cc5)c5ccc(-c6cccc7c6ccc6ccccc67)cc5)cc4)ccc32)cc1 ; c1ccc(-n2c3ccccc3c3c(-c4ccc(N(c5ccc(-c6cccc(-c7ccc8ccccc8c7)c6)cc5)c5ccc(-c6cccc7c6ccc6ccccc67)cc5)cc4)cccc32)cc1 ; c1ccc(-n2c3ccccc3c3cc(-c4cccc(N(c5ccc(-c6cccc(-c7ccc8ccccc8c7)c6)cc5)c5ccc(-c6cccc7c6ccc6ccccc67)cc5)c4)ccc32)cc1]

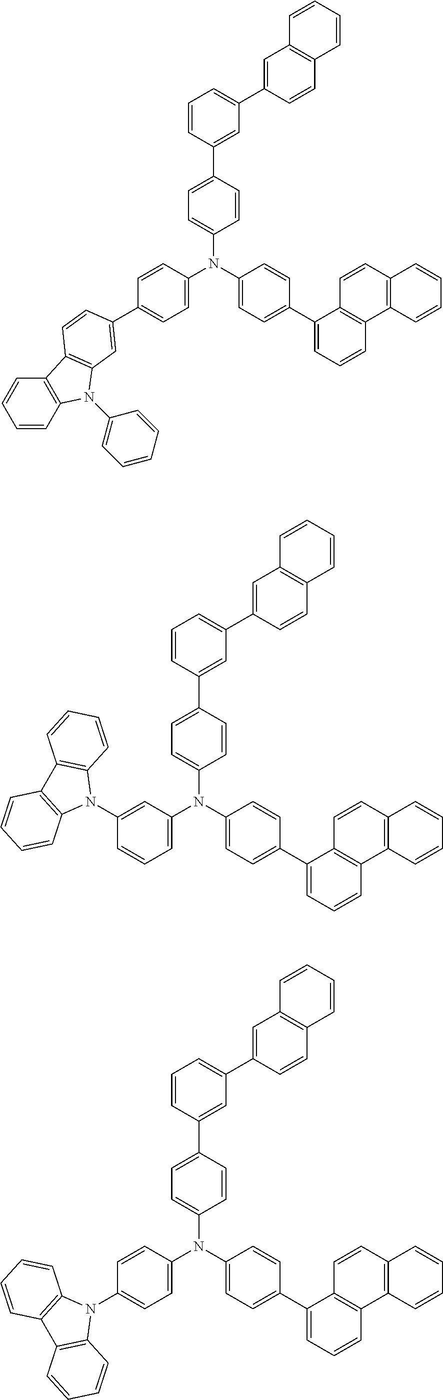 [SMILES: c1ccc(-n2c3ccccc3c3ccc(-c4ccc(N(c5ccc(-c6cccc(-c7ccc8ccccc8c7)c6)cc5)c5ccc(-c6cccc7c6ccc6ccccc67)cc5)cc4)cc32)cc1 ; c1cc(-c2ccc(N(c3ccc(-c4cccc5c4ccc4ccccc45)cc3)c3cccc(-n4c5ccccc5c5ccccc54)c3)cc2)cc(-c2ccc3ccccc3c2)c1 ; c1cc(-c2ccc(N(c3ccc(-c4cccc5c4ccc4ccccc45)cc3)c3ccc(-n4c5ccccc5c5ccccc54)cc3)cc2)cc(-c2ccc3ccccc3c2)c1]

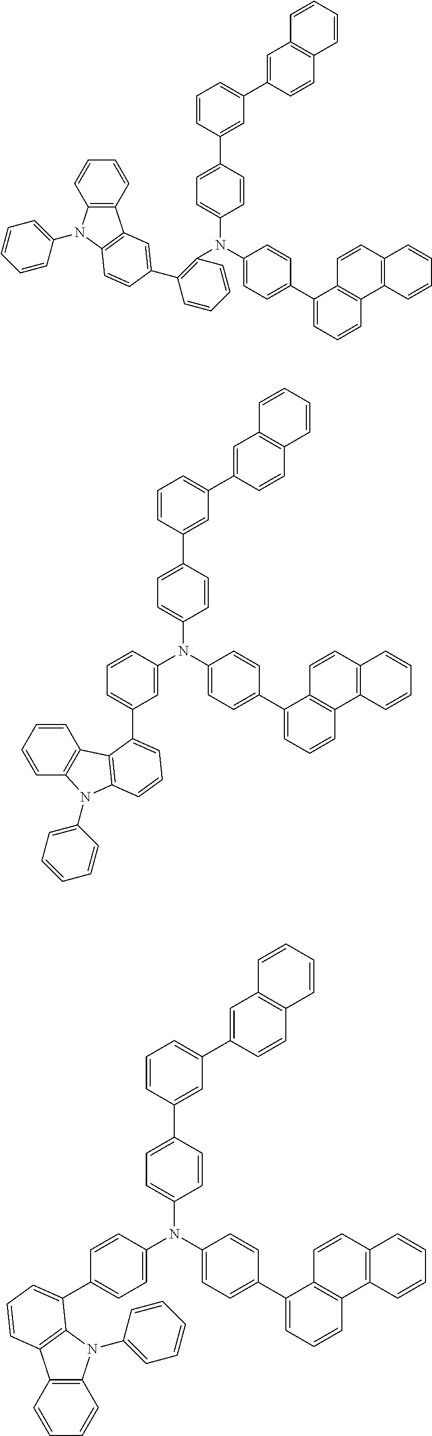 [SMILES: c1ccc(-n2c3ccccc3c3cc(-c4ccccc4N(c4ccc(-c5cccc(-c6ccc7ccccc7c6)c5)cc4)c4ccc(-c5cccc6c5ccc5ccccc56)cc4)ccc32)cc1 ; c1ccc(-n2c3ccccc3c3c(-c4cccc(N(c5ccc(-c6cccc(-c7ccc8ccccc8c7)c6)cc5)c5ccc(-c6cccc7c6ccc6ccccc67)cc5)c4)cccc32)cc1 ; c1ccc(-n2c3ccccc3c3cccc(-c4ccc(N(c5ccc(-c6cccc(-c7ccc8ccccc8c7)c6)cc5)c5ccc(-c6cccc7c6ccc6ccccc67)cc5)cc4)c32)cc1]

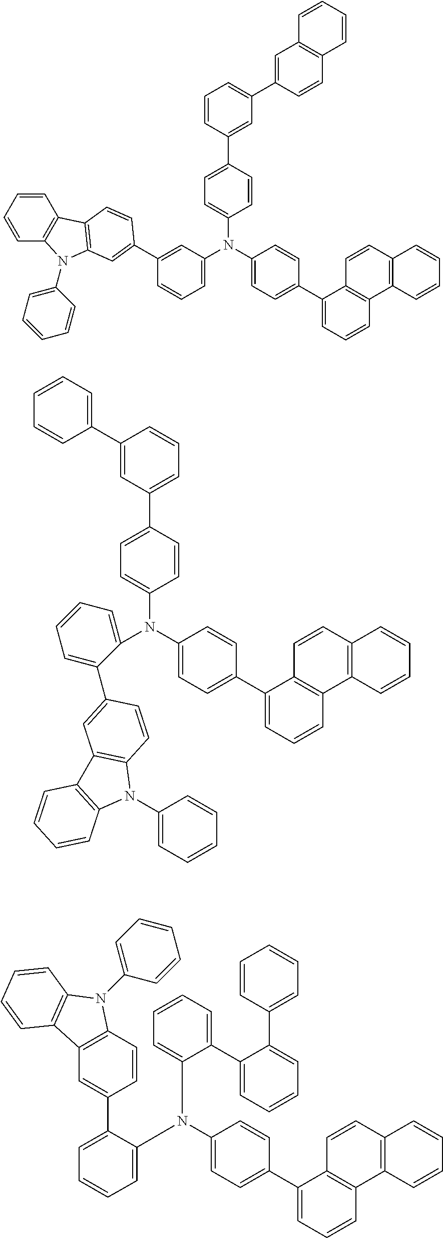

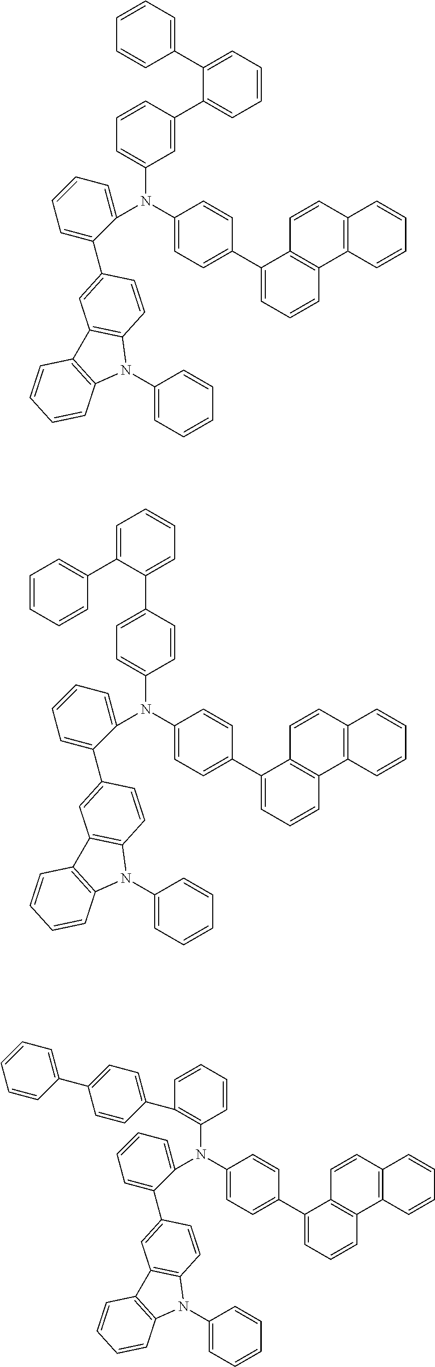 [SMILES: c1ccc(-c2ccccc2-c2cccc(N(c3ccc(-c4cccc5c4ccc4ccccc45)cc3)c3ccccc3-c3ccc4c(c3)c3ccccc3n4-c3ccccc3)c2)cc1 ; c1ccc(-c2ccccc2-c2ccc(N(c3ccc(-c4cccc5c4ccc4ccccc45)cc3)c3ccccc3-c3ccc4c(c3)c3ccccc3n4-c3ccccc3)cc2)cc1 ; c1ccc(-c2ccc(-c3ccccc3N(c3ccc(-c4cccc5c4ccc4ccccc45)cc3)c3ccccc3-c3ccc4c(c3)c3ccccc3n4-c3ccccc3)cc2)cc1]

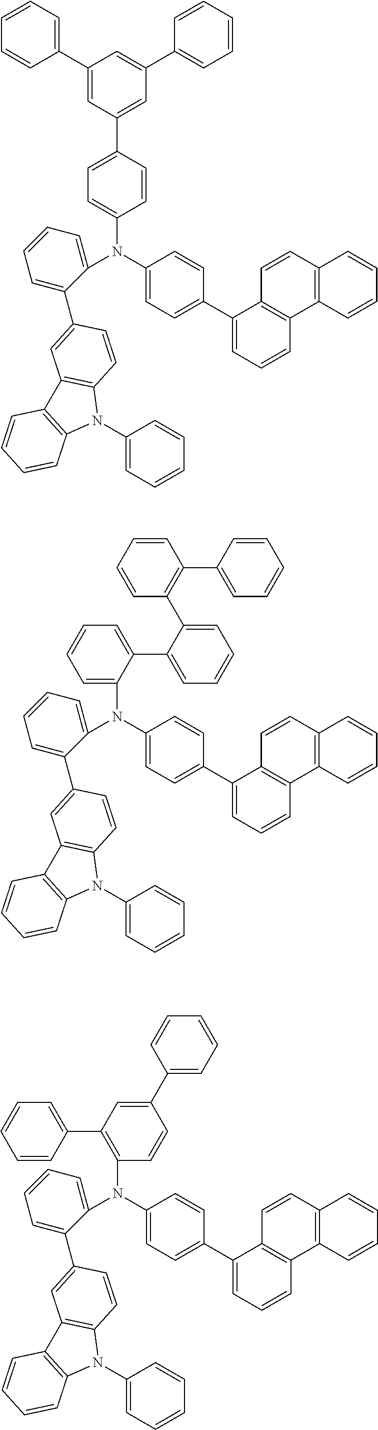 [SMILES: c1ccc(-c2cc(-c3ccccc3)cc(-c3ccc(N(c4ccc(-c5cccc6c5ccc5ccccc56)cc4)c4ccccc4-c4ccc5c(c4)c4ccccc4n5-c4ccccc4)cc3)c2)cc1 ; c1ccc(-c2ccccc2-c2ccccc2-c2ccccc2N(c2ccc(-c3cccc4c3ccc3ccccc34)cc2)c2ccccc2-c2ccc3c(c2)c2ccccc2n3-c2ccccc2)cc1 ; c1ccc(-c2ccc(N(c3ccc(-c4cccc5c4ccc4ccccc45)cc3)c3ccccc3-c3ccc4c(c3)c3ccccc3n4-c3ccccc3)c(-c3ccccc3)c2)cc1]

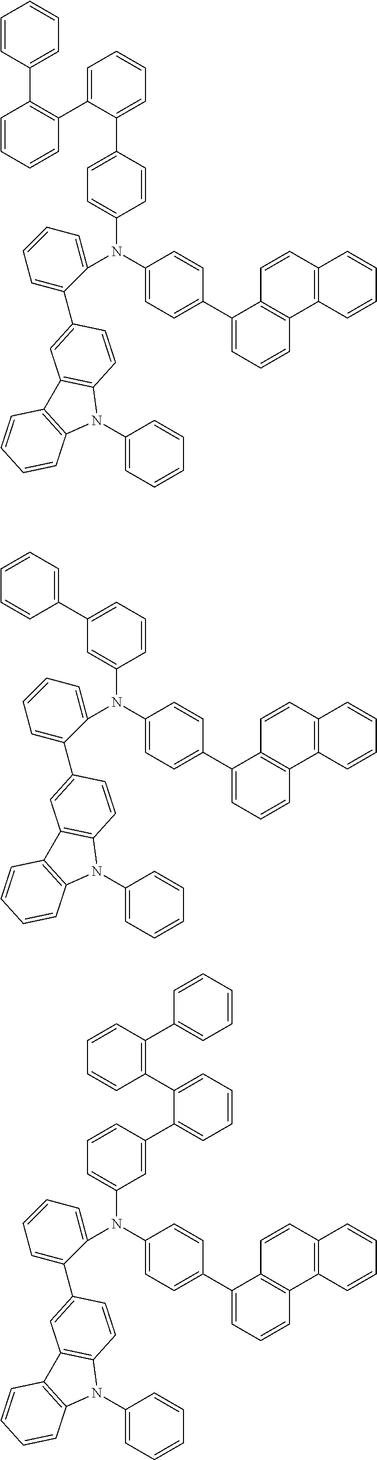 [SMILES: c1ccc(-c2ccccc2-c2ccccc2-c2ccc(N(c3ccc(-c4cccc5c4ccc4ccccc45)cc3)c3ccccc3-c3ccc4c(c3)c3ccccc3n4-c3ccccc3)cc2)cc1 ; c1ccc(-c2cccc(N(c3ccc(-c4cccc5c4ccc4ccccc45)cc3)c3ccccc3-c3ccc4c(c3)c3ccccc3n4-c3ccccc3)c2)cc1 ; c1ccc(-c2ccccc2-c2ccccc2-c2cccc(N(c3ccc(-c4cccc5c4ccc4ccccc45)cc3)c3ccccc3-c3ccc4c(c3)c3ccccc3n4-c3ccccc3)c2)cc1]

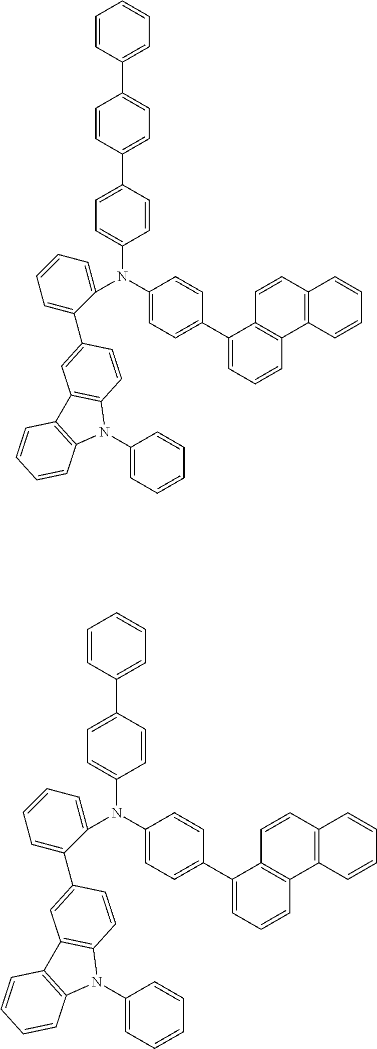 [SMILES: c1ccc(-c2ccc(-c3ccc(N(c4ccc(-c5cccc6c5ccc5ccccc56)cc4)c4ccccc4-c4ccc5c(c4)c4ccccc4n5-c4ccccc4)cc3)cc2)cc1 ; c1ccc(-c2ccc(N(c3ccc(-c4cccc5c4ccc4ccccc45)cc3)c3ccccc3-c3ccc4c(c3)c3ccccc3n4-c3ccccc3)cc2)cc1]

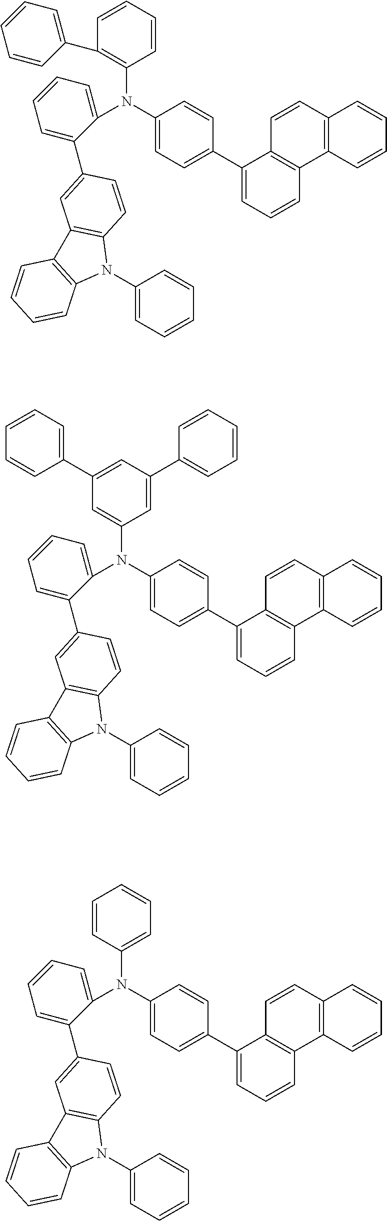 [SMILES: c1ccc(-c2ccccc2N(c2ccc(-c3cccc4c3ccc3ccccc34)cc2)c2ccccc2-c2ccc3c(c2)c2ccccc2n3-c2ccccc2)cc1 ; c1ccc(-c2cc(-c3ccccc3)cc(N(c3ccc(-c4cccc5c4ccc4ccccc45)cc3)c3ccccc3-c3ccc4c(c3)c3ccccc3n4-c3ccccc3)c2)cc1 ; c1ccc(N(c2ccc(-c3cccc4c3ccc3ccccc34)cc2)c2ccccc2-c2ccc3c(c2)c2ccccc2n3-c2ccccc2)cc1]

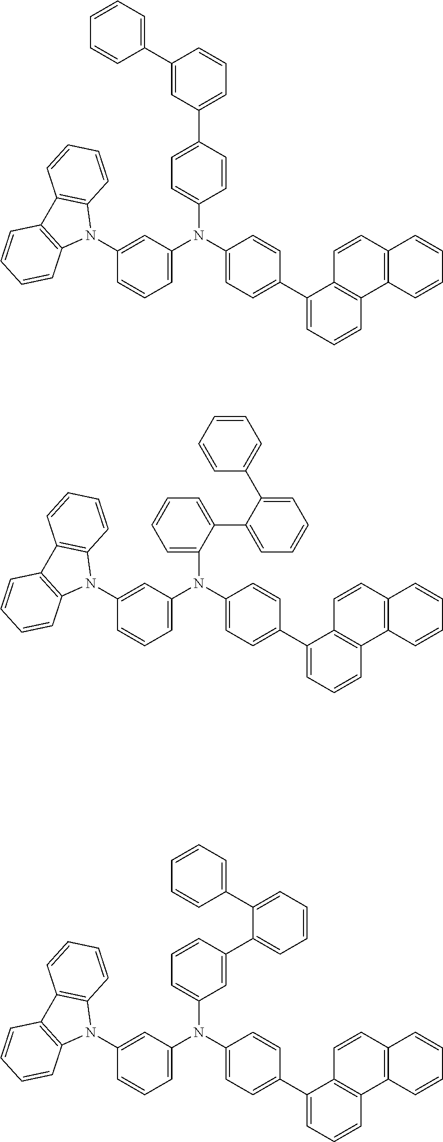 [SMILES: c1ccc(-c2cccc(-c3ccc(N(c4ccc(-c5cccc6c5ccc5ccccc56)cc4)c4cccc(-n5c6ccccc6c6ccccc65)c4)cc3)c2)cc1 ; c1ccc(-c2ccccc2-c2ccccc2N(c2ccc(-c3cccc4c3ccc3ccccc34)cc2)c2cccc(-n3c4ccccc4c4ccccc43)c2)cc1 ; c1ccc(-c2ccccc2-c2cccc(N(c3ccc(-c4cccc5c4ccc4ccccc45)cc3)c3cccc(-n4c5ccccc5c5ccccc54)c3)c2)cc1]

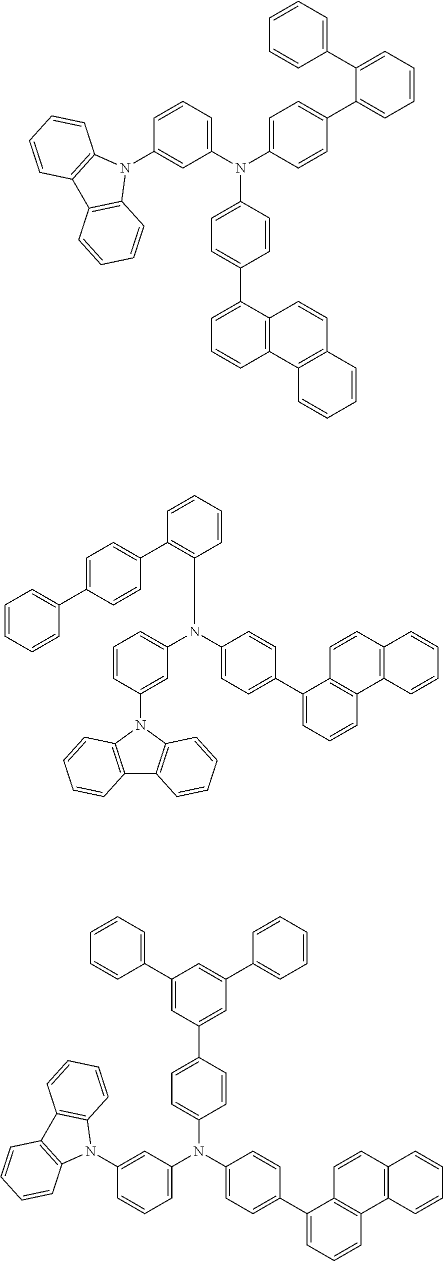 [SMILES: c1ccc(-c2ccccc2-c2ccc(N(c3ccc(-c4cccc5c4ccc4ccccc45)cc3)c3cccc(-n4c5ccccc5c5ccccc54)c3)cc2)cc1 ; c1ccc(-c2ccc(-c3ccccc3N(c3ccc(-c4cccc5c4ccc4ccccc45)cc3)c3cccc(-n4c5ccccc5c5ccccc54)c3)cc2)cc1 ; c1ccc(-c2cc(-c3ccccc3)cc(-c3ccc(N(c4ccc(-c5cccc6c5ccc5ccccc56)cc4)c4cccc(-n5c6ccccc6c6ccccc65)c4)cc3)c2)cc1]

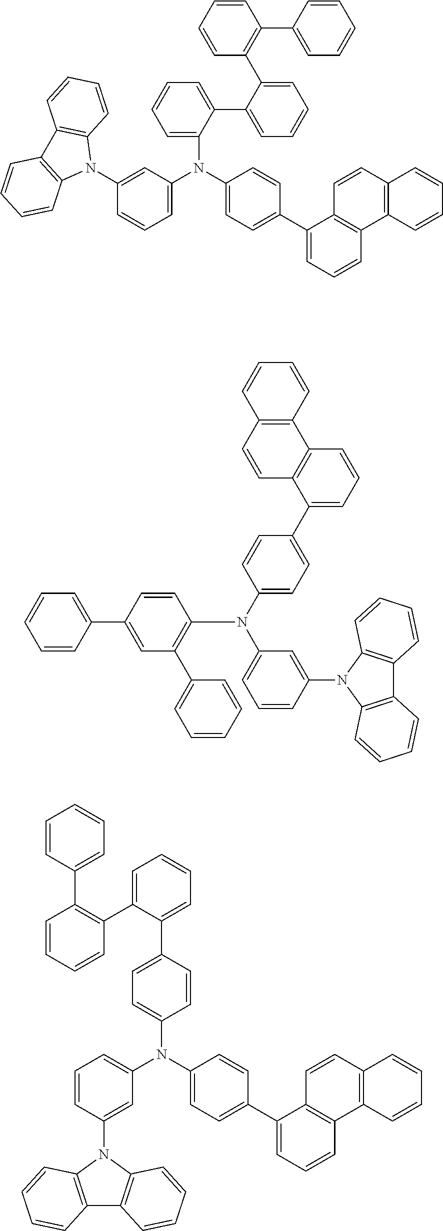 [SMILES: c1ccc(-c2ccccc2-c2ccccc2-c2ccccc2N(c2ccc(-c3cccc4c3ccc3ccccc34)cc2)c2cccc(-n3c4ccccc4c4ccccc43)c2)cc1 ; c1ccc(-c2ccc(N(c3ccc(-c4cccc5c4ccc4ccccc45)cc3)c3cccc(-n4c5ccccc5c5ccccc54)c3)c(-c3ccccc3)c2)cc1 ; c1ccc(-c2ccccc2-c2ccccc2-c2ccc(N(c3ccc(-c4cccc5c4ccc4ccccc45)cc3)c3cccc(-n4c5ccccc5c5ccccc54)c3)cc2)cc1]

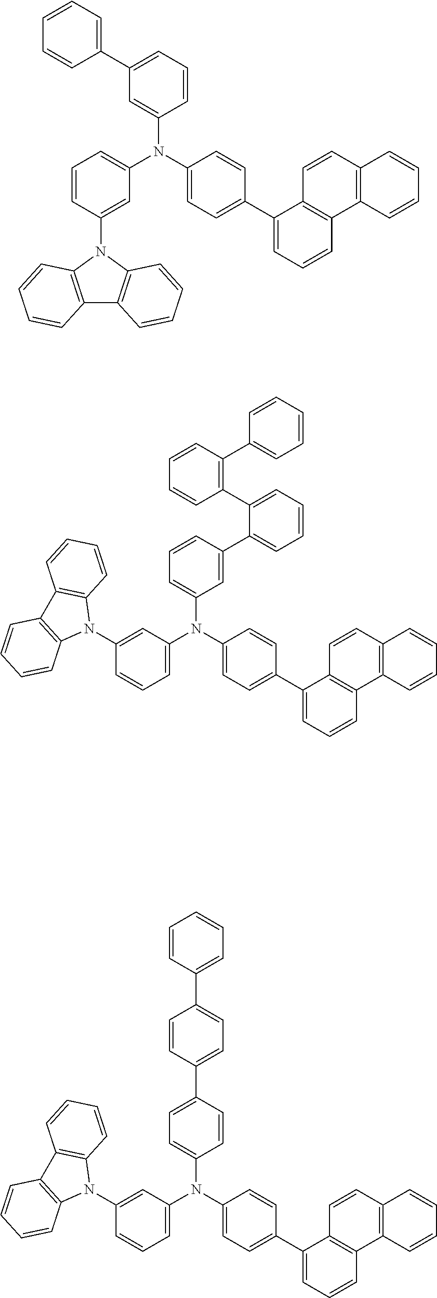 [SMILES: c1ccc(-c2cccc(N(c3ccc(-c4cccc5c4ccc4ccccc45)cc3)c3cccc(-n4c5ccccc5c5ccccc54)c3)c2)cc1 ; c1ccc(-c2ccccc2-c2ccccc2-c2cccc(N(c3ccc(-c4cccc5c4ccc4ccccc45)cc3)c3cccc(-n4c5ccccc5c5ccccc54)c3)c2)cc1 ; c1ccc(-c2ccc(-c3ccc(N(c4ccc(-c5cccc6c5ccc5ccccc56)cc4)c4cccc(-n5c6ccccc6c6ccccc65)c4)cc3)cc2)cc1]

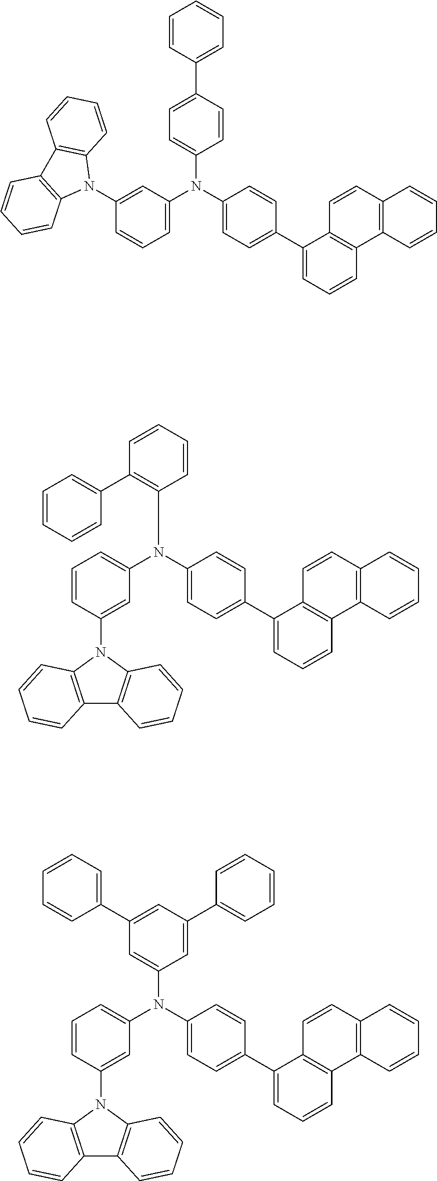 [SMILES: c1ccc(-c2ccc(N(c3ccc(-c4cccc5c4ccc4ccccc45)cc3)c3cccc(-n4c5ccccc5c5ccccc54)c3)cc2)cc1 ; c1ccc(-c2ccccc2N(c2ccc(-c3cccc4c3ccc3ccccc34)cc2)c2cccc(-n3c4ccccc4c4ccccc43)c2)cc1 ; c1ccc(-c2cc(-c3ccccc3)cc(N(c3ccc(-c4cccc5c4ccc4ccccc45)cc3)c3cccc(-n4c5ccccc5c5ccccc54)c3)c2)cc1]

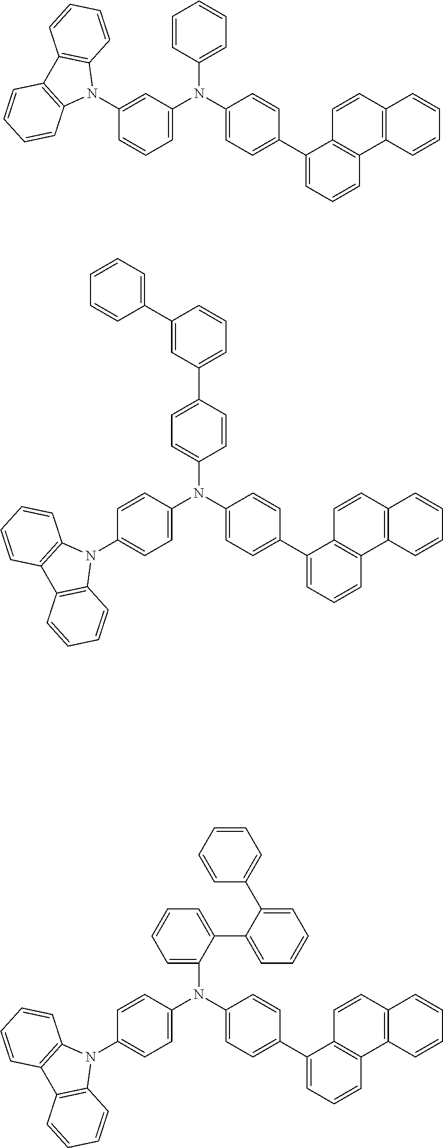 [SMILES: c1ccc(N(c2ccc(-c3cccc4c3ccc3ccccc34)cc2)c2cccc(-n3c4ccccc4c4ccccc43)c2)cc1 ; c1ccc(-c2cccc(-c3ccc(N(c4ccc(-c5cccc6c5ccc5ccccc56)cc4)c4ccc(-n5c6ccccc6c6ccccc65)cc4)cc3)c2)cc1 ; c1ccc(-c2ccccc2-c2ccccc2N(c2ccc(-c3cccc4c3ccc3ccccc34)cc2)c2ccc(-n3c4ccccc4c4ccccc43)cc2)cc1]

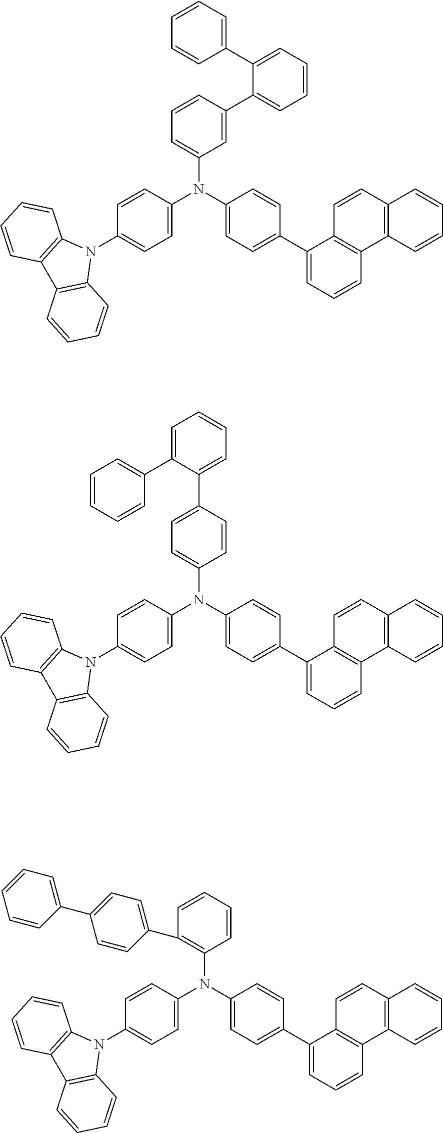 [SMILES: c1ccc(-c2ccccc2-c2cccc(N(c3ccc(-c4cccc5c4ccc4ccccc45)cc3)c3ccc(-n4c5ccccc5c5ccccc54)cc3)c2)cc1 ; c1ccc(-c2ccccc2-c2ccc(N(c3ccc(-c4cccc5c4ccc4ccccc45)cc3)c3ccc(-n4c5ccccc5c5ccccc54)cc3)cc2)cc1 ; c1ccc(-c2ccc(-c3ccccc3N(c3ccc(-c4cccc5c4ccc4ccccc45)cc3)c3ccc(-n4c5ccccc5c5ccccc54)cc3)cc2)cc1]

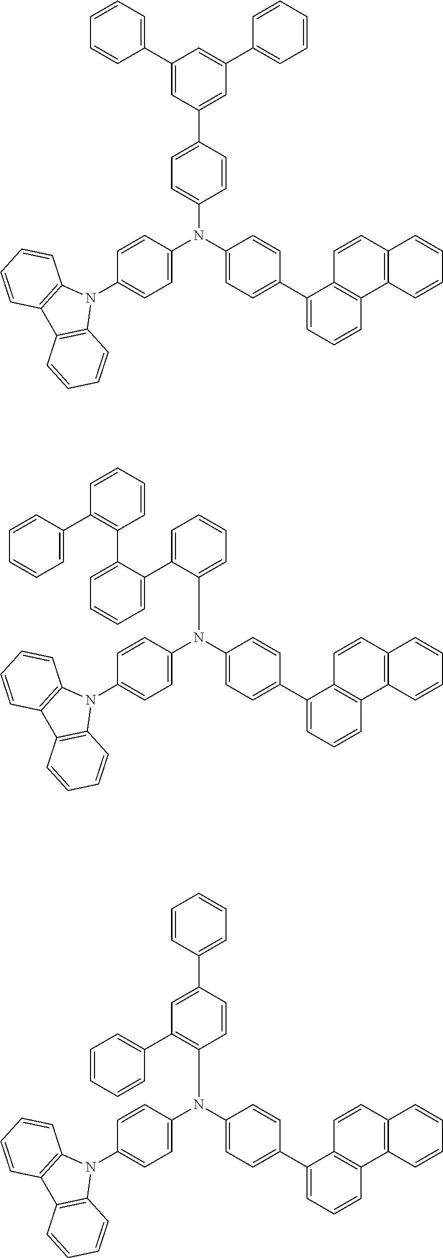 [SMILES: c1ccc(-c2cc(-c3ccccc3)cc(-c3ccc(N(c4ccc(-c5cccc6c5ccc5ccccc56)cc4)c4ccc(-n5c6ccccc6c6ccccc65)cc4)cc3)c2)cc1 ; c1ccc(-c2ccccc2-c2ccccc2-c2ccccc2N(c2ccc(-c3cccc4c3ccc3ccccc34)cc2)c2ccc(-n3c4ccccc4c4ccccc43)cc2)cc1 ; c1ccc(-c2ccc(N(c3ccc(-c4cccc5c4ccc4ccccc45)cc3)c3ccc(-n4c5ccccc5c5ccccc54)cc3)c(-c3ccccc3)c2)cc1]

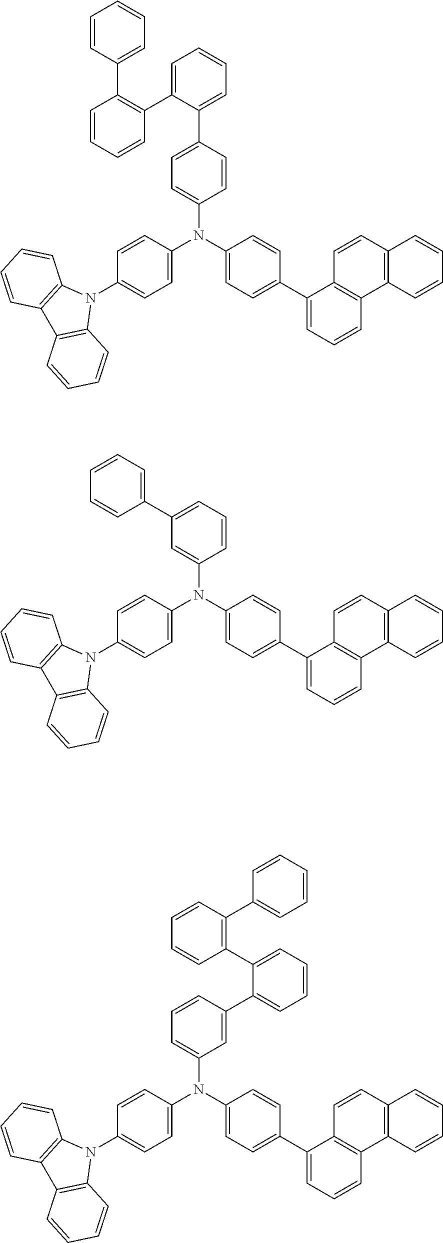 [SMILES: c1ccc(-c2ccccc2-c2ccccc2-c2ccc(N(c3ccc(-c4cccc5c4ccc4ccccc45)cc3)c3ccc(-n4c5ccccc5c5ccccc54)cc3)cc2)cc1 ; c1ccc(-c2cccc(N(c3ccc(-c4cccc5c4ccc4ccccc45)cc3)c3ccc(-n4c5ccccc5c5ccccc54)cc3)c2)cc1 ; c1ccc(-c2ccccc2-c2ccccc2-c2cccc(N(c3ccc(-c4cccc5c4ccc4ccccc45)cc3)c3ccc(-n4c5ccccc5c5ccccc54)cc3)c2)cc1]

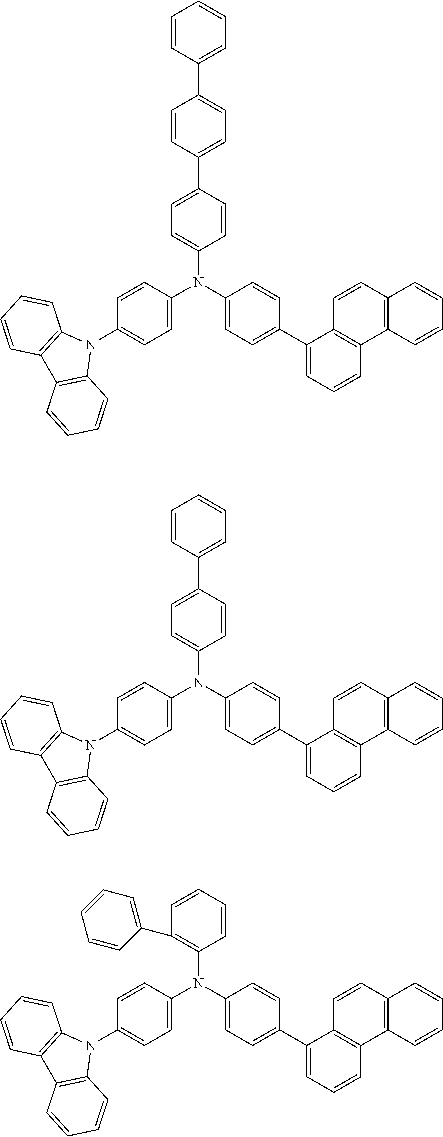 [SMILES: c1ccc(-c2ccc(-c3ccc(N(c4ccc(-c5cccc6c5ccc5ccccc56)cc4)c4ccc(-n5c6ccccc6c6ccccc65)cc4)cc3)cc2)cc1 ; c1ccc(-c2ccc(N(c3ccc(-c4cccc5c4ccc4ccccc45)cc3)c3ccc(-n4c5ccccc5c5ccccc54)cc3)cc2)cc1 ; c1ccc(-c2ccccc2N(c2ccc(-c3cccc4c3ccc3ccccc34)cc2)c2ccc(-n3c4ccccc4c4ccccc43)cc2)cc1]

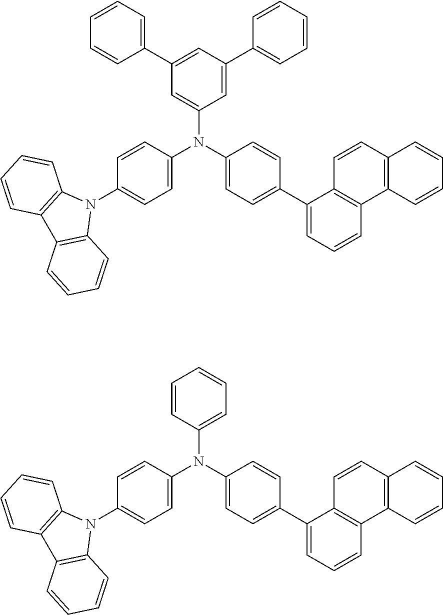 [SMILES: c1ccc(-c2cc(-c3ccccc3)cc(N(c3ccc(-c4cccc5c4ccc4ccccc45)cc3)c3ccc(-n4c5ccccc5c5ccccc54)cc3)c2)cc1 ; c1ccc(N(c2ccc(-c3cccc4c3ccc3ccccc34)cc2)c2ccc(-n3c4ccccc4c4ccccc43)cc2)cc1]

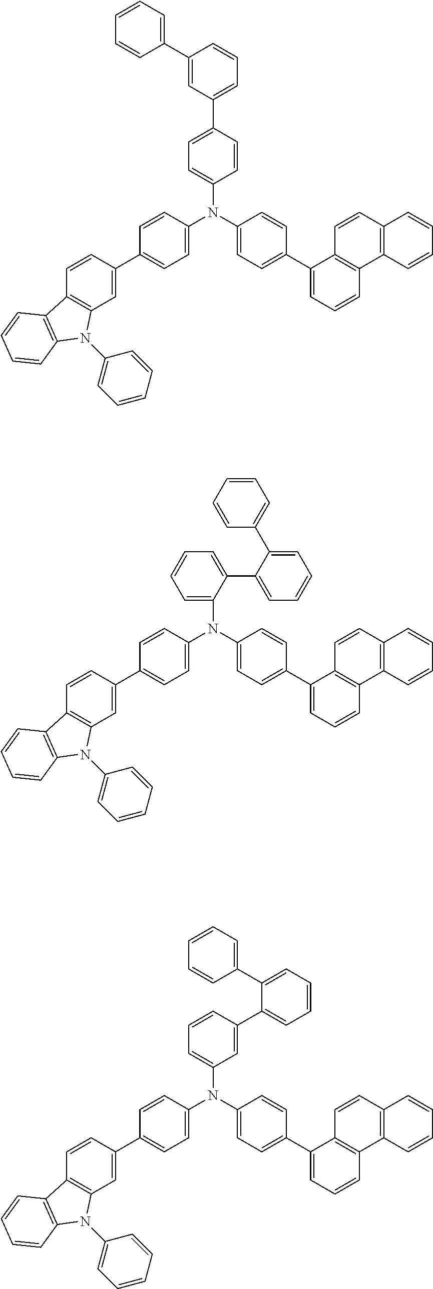 [SMILES: c1ccc(-c2cccc(-c3ccc(N(c4ccc(-c5ccc6c7ccccc7n(-c7ccccc7)c6c5)cc4)c4ccc(-c5cccc6c5ccc5ccccc56)cc4)cc3)c2)cc1 ; c1ccc(-c2ccccc2-c2ccccc2N(c2ccc(-c3ccc4c5ccccc5n(-c5ccccc5)c4c3)cc2)c2ccc(-c3cccc4c3ccc3ccccc34)cc2)cc1 ; c1ccc(-c2ccccc2-c2cccc(N(c3ccc(-c4ccc5c6ccccc6n(-c6ccccc6)c5c4)cc3)c3ccc(-c4cccc5c4ccc4ccccc45)cc3)c2)cc1]

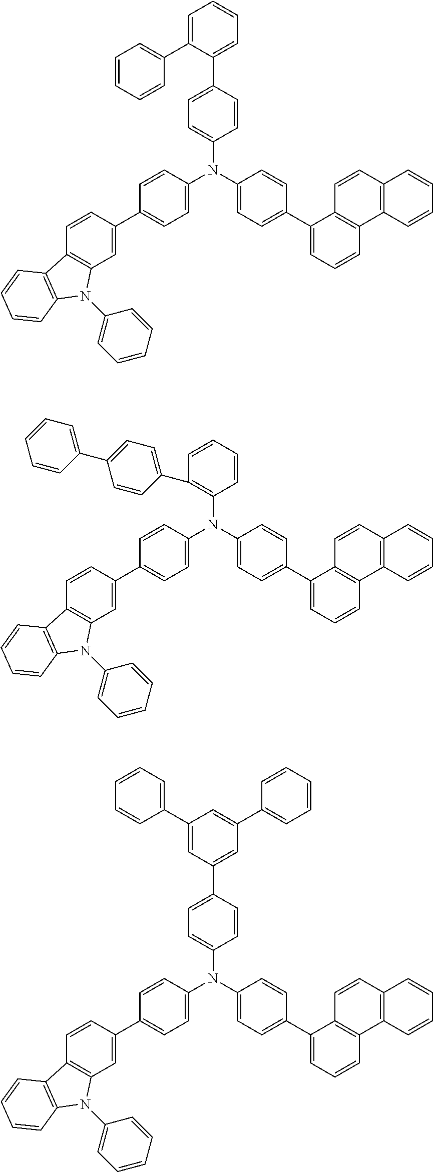 [SMILES: c1ccc(-c2ccccc2-c2ccc(N(c3ccc(-c4ccc5c6ccccc6n(-c6ccccc6)c5c4)cc3)c3ccc(-c4cccc5c4ccc4ccccc45)cc3)cc2)cc1 ; c1ccc(-c2ccc(-c3ccccc3N(c3ccc(-c4ccc5c6ccccc6n(-c6ccccc6)c5c4)cc3)c3ccc(-c4cccc5c4ccc4ccccc45)cc3)cc2)cc1 ; c1ccc(-c2cc(-c3ccccc3)cc(-c3ccc(N(c4ccc(-c5ccc6c7ccccc7n(-c7ccccc7)c6c5)cc4)c4ccc(-c5cccc6c5ccc5ccccc56)cc4)cc3)c2)cc1]

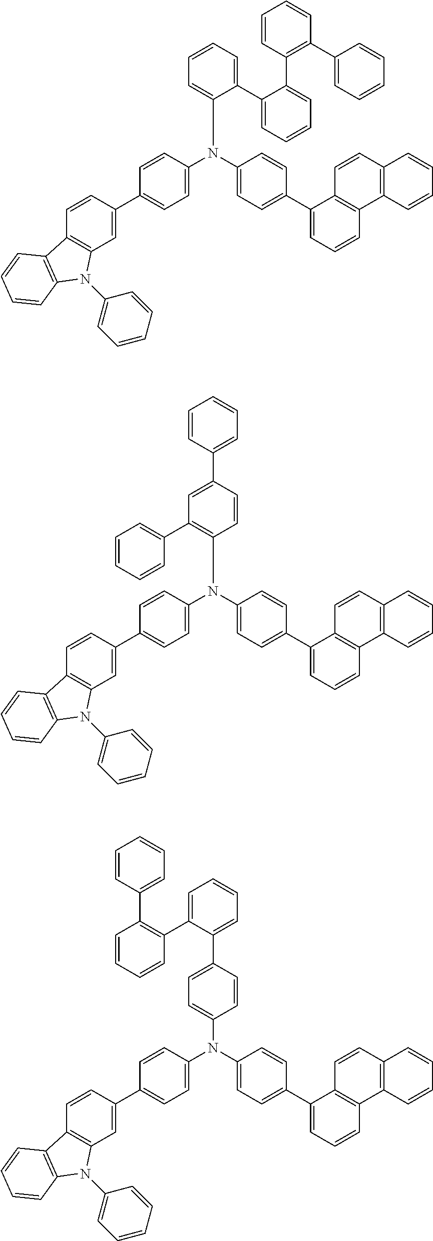 [SMILES: c1ccc(-c2ccccc2-c2ccccc2-c2ccccc2N(c2ccc(-c3ccc4c5ccccc5n(-c5ccccc5)c4c3)cc2)c2ccc(-c3cccc4c3ccc3ccccc34)cc2)cc1 ; c1ccc(-c2ccc(N(c3ccc(-c4ccc5c6ccccc6n(-c6ccccc6)c5c4)cc3)c3ccc(-c4cccc5c4ccc4ccccc45)cc3)c(-c3ccccc3)c2)cc1 ; c1ccc(-c2ccccc2-c2ccccc2-c2ccc(N(c3ccc(-c4ccc5c6ccccc6n(-c6ccccc6)c5c4)cc3)c3ccc(-c4cccc5c4ccc4ccccc45)cc3)cc2)cc1]

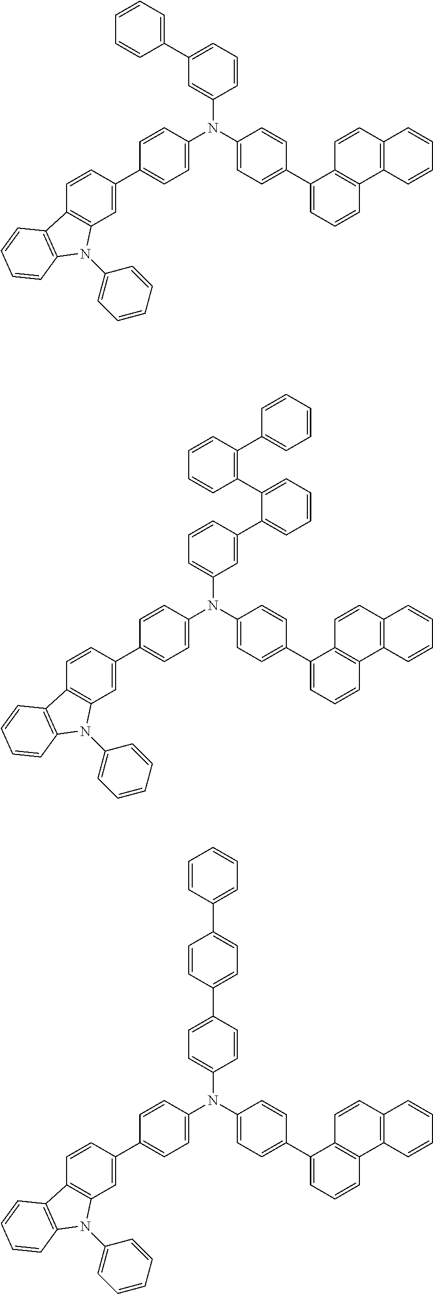 [SMILES: c1ccc(-c2cccc(N(c3ccc(-c4ccc5c6ccccc6n(-c6ccccc6)c5c4)cc3)c3ccc(-c4cccc5c4ccc4ccccc45)cc3)c2)cc1 ; c1ccc(-c2ccccc2-c2ccccc2-c2cccc(N(c3ccc(-c4ccc5c6ccccc6n(-c6ccccc6)c5c4)cc3)c3ccc(-c4cccc5c4ccc4ccccc45)cc3)c2)cc1 ; c1ccc(-c2ccc(-c3ccc(N(c4ccc(-c5ccc6c7ccccc7n(-c7ccccc7)c6c5)cc4)c4ccc(-c5cccc6c5ccc5ccccc56)cc4)cc3)cc2)cc1]

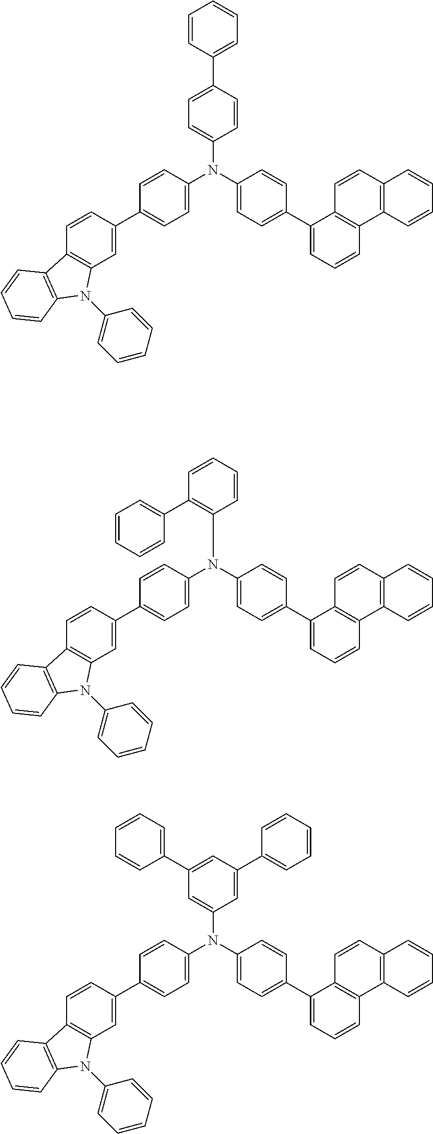 [SMILES: c1ccc(-c2ccc(N(c3ccc(-c4ccc5c6ccccc6n(-c6ccccc6)c5c4)cc3)c3ccc(-c4cccc5c4ccc4ccccc45)cc3)cc2)cc1 ; c1ccc(-c2ccccc2N(c2ccc(-c3ccc4c5ccccc5n(-c5ccccc5)c4c3)cc2)c2ccc(-c3cccc4c3ccc3ccccc34)cc2)cc1 ; c1ccc(-c2cc(-c3ccccc3)cc(N(c3ccc(-c4ccc5c6ccccc6n(-c6ccccc6)c5c4)cc3)c3ccc(-c4cccc5c4ccc4ccccc45)cc3)c2)cc1]

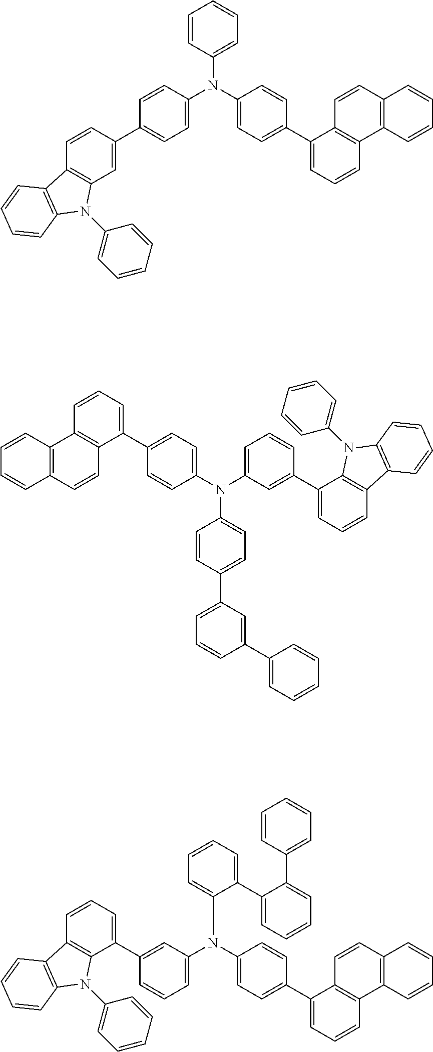 [SMILES: c1ccc(N(c2ccc(-c3ccc4c5ccccc5n(-c5ccccc5)c4c3)cc2)c2ccc(-c3cccc4c3ccc3ccccc34)cc2)cc1 ; c1ccc(-c2cccc(-c3ccc(N(c4ccc(-c5cccc6c5ccc5ccccc56)cc4)c4cccc(-c5cccc6c7ccccc7n(-c7ccccc7)c56)c4)cc3)c2)cc1 ; c1ccc(-c2ccccc2-c2ccccc2N(c2ccc(-c3cccc4c3ccc3ccccc34)cc2)c2cccc(-c3cccc4c5ccccc5n(-c5ccccc5)c34)c2)cc1]

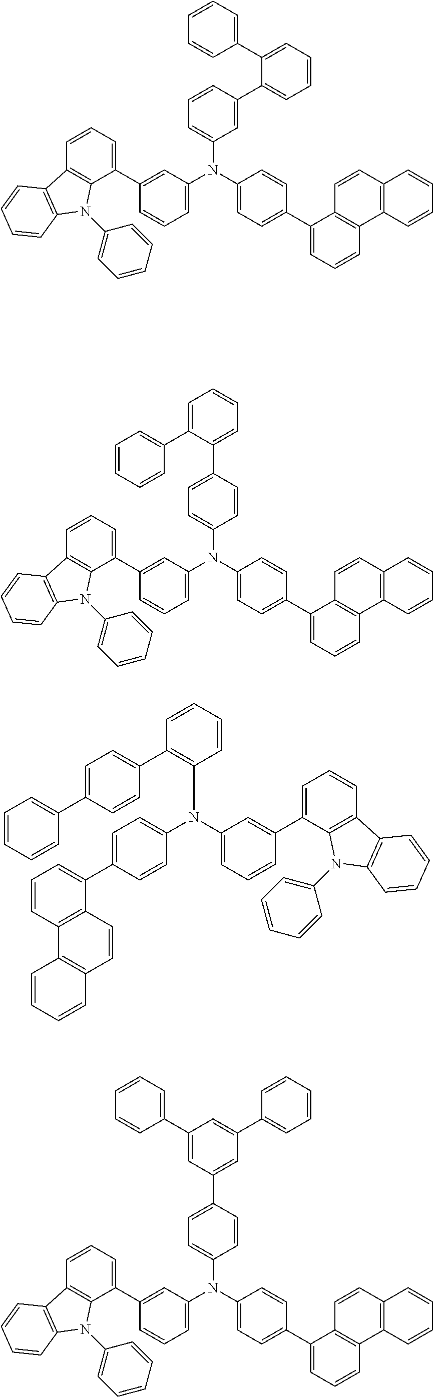 [SMILES: c1ccc(-c2ccccc2-c2cccc(N(c3ccc(-c4cccc5c4ccc4ccccc45)cc3)c3cccc(-c4cccc5c6ccccc6n(-c6ccccc6)c45)c3)c2)cc1 ; c1ccc(-c2ccccc2-c2ccc(N(c3ccc(-c4cccc5c4ccc4ccccc45)cc3)c3cccc(-c4cccc5c6ccccc6n(-c6ccccc6)c45)c3)cc2)cc1 ; c1ccc(-c2ccc(-c3ccccc3N(c3ccc(-c4cccc5c4ccc4ccccc45)cc3)c3cccc(-c4cccc5c6ccccc6n(-c6ccccc6)c45)c3)cc2)cc1 ; c1ccc(-c2cc(-c3ccccc3)cc(-c3ccc(N(c4ccc(-c5cccc6c5ccc5ccccc56)cc4)c4cccc(-c5cccc6c7ccccc7n(-c7ccccc7)c56)c4)cc3)c2)cc1]

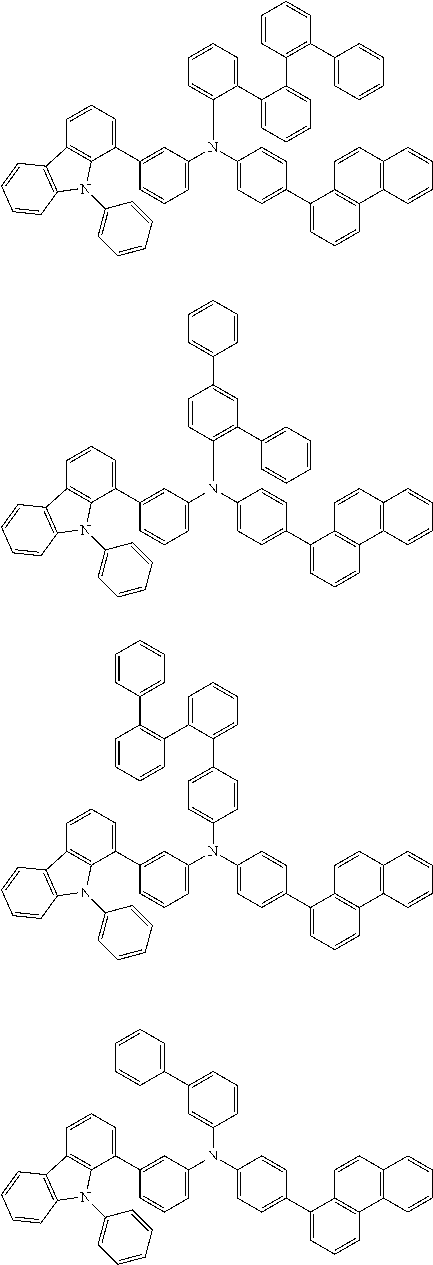 [SMILES: c1ccc(-c2ccccc2-c2ccccc2-c2ccccc2N(c2ccc(-c3cccc4c3ccc3ccccc34)cc2)c2cccc(-c3cccc4c5ccccc5n(-c5ccccc5)c34)c2)cc1 ; c1ccc(-c2ccc(N(c3ccc(-c4cccc5c4ccc4ccccc45)cc3)c3cccc(-c4cccc5c6ccccc6n(-c6ccccc6)c45)c3)c(-c3ccccc3)c2)cc1 ; c1ccc(-c2ccccc2-c2ccccc2-c2ccc(N(c3ccc(-c4cccc5c4ccc4ccccc45)cc3)c3cccc(-c4cccc5c6ccccc6n(-c6ccccc6)c45)c3)cc2)cc1 ; c1ccc(-c2cccc(N(c3ccc(-c4cccc5c4ccc4ccccc45)cc3)c3cccc(-c4cccc5c6ccccc6n(-c6ccccc6)c45)c3)c2)cc1]

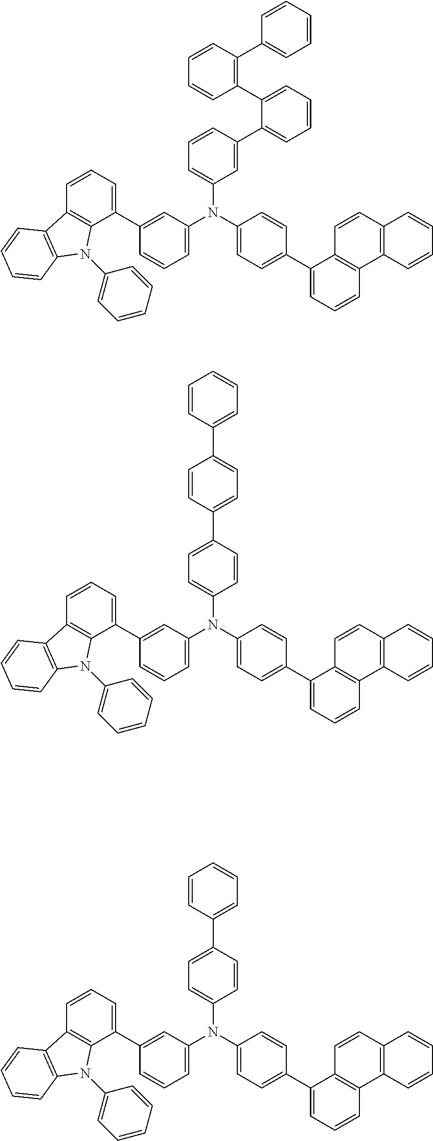 [SMILES: c1ccc(-c2ccccc2-c2ccccc2-c2cccc(N(c3ccc(-c4cccc5c4ccc4ccccc45)cc3)c3cccc(-c4cccc5c6ccccc6n(-c6ccccc6)c45)c3)c2)cc1 ; c1ccc(-c2ccc(-c3ccc(N(c4ccc(-c5cccc6c5ccc5ccccc56)cc4)c4cccc(-c5cccc6c7ccccc7n(-c7ccccc7)c56)c4)cc3)cc2)cc1 ; c1ccc(-c2ccc(N(c3ccc(-c4cccc5c4ccc4ccccc45)cc3)c3cccc(-c4cccc5c6ccccc6n(-c6ccccc6)c45)c3)cc2)cc1]

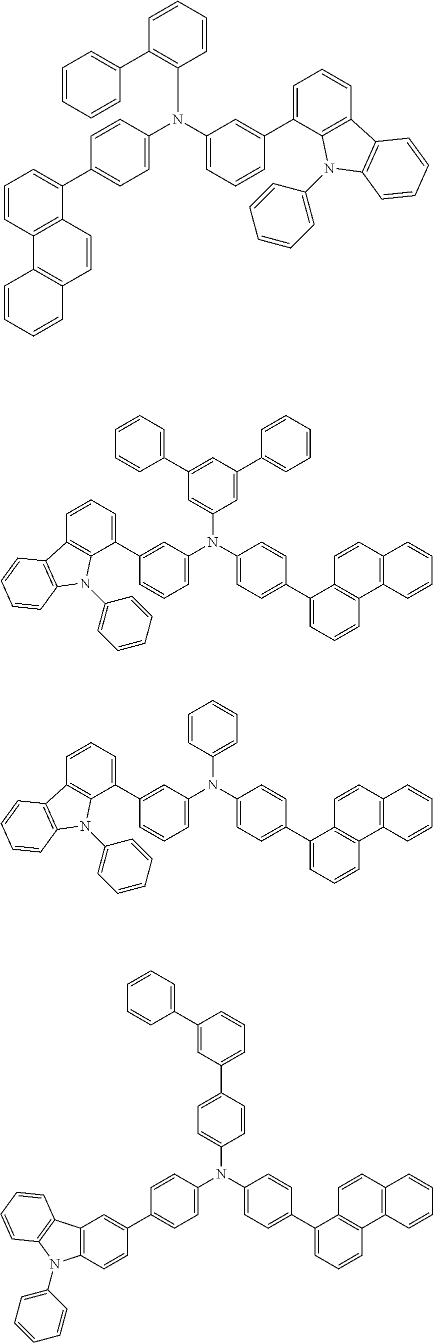 [SMILES: c1ccc(-c2ccccc2N(c2ccc(-c3cccc4c3ccc3ccccc34)cc2)c2cccc(-c3cccc4c5ccccc5n(-c5ccccc5)c34)c2)cc1 ; c1ccc(-c2cc(-c3ccccc3)cc(N(c3ccc(-c4cccc5c4ccc4ccccc45)cc3)c3cccc(-c4cccc5c6ccccc6n(-c6ccccc6)c45)c3)c2)cc1 ; c1ccc(N(c2ccc(-c3cccc4c3ccc3ccccc34)cc2)c2cccc(-c3cccc4c5ccccc5n(-c5ccccc5)c34)c2)cc1 ; c1ccc(-c2cccc(-c3ccc(N(c4ccc(-c5ccc6c(c5)c5ccccc5n6-c5ccccc5)cc4)c4ccc(-c5cccc6c5ccc5ccccc56)cc4)cc3)c2)cc1]

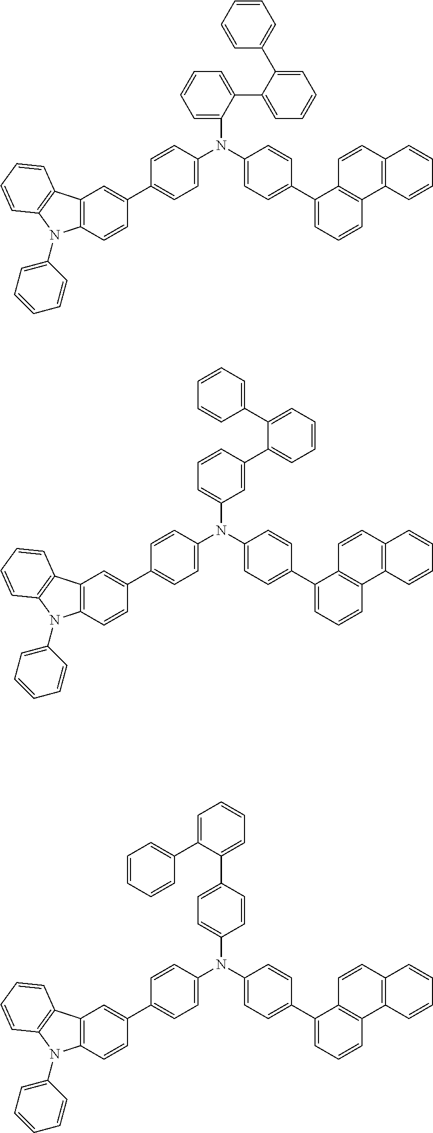 [SMILES: c1ccc(-c2ccccc2-c2ccccc2N(c2ccc(-c3ccc4c(c3)c3ccccc3n4-c3ccccc3)cc2)c2ccc(-c3cccc4c3ccc3ccccc34)cc2)cc1 ; c1ccc(-c2ccccc2-c2cccc(N(c3ccc(-c4ccc5c(c4)c4ccccc4n5-c4ccccc4)cc3)c3ccc(-c4cccc5c4ccc4ccccc45)cc3)c2)cc1 ; c1ccc(-c2ccccc2-c2ccc(N(c3ccc(-c4ccc5c(c4)c4ccccc4n5-c4ccccc4)cc3)c3ccc(-c4cccc5c4ccc4ccccc45)cc3)cc2)cc1]

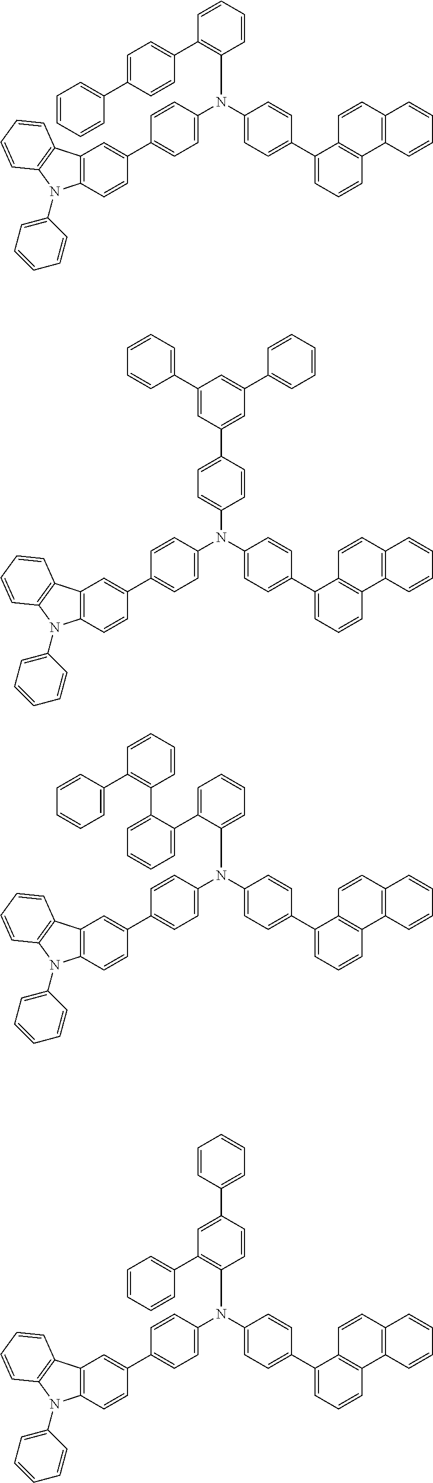

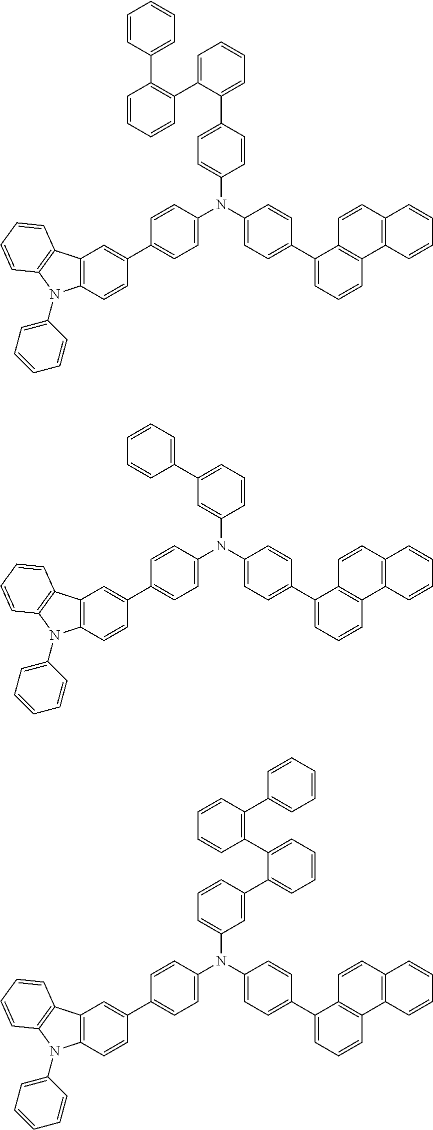 [SMILES: c1ccc(-c2ccccc2-c2ccccc2-c2ccc(N(c3ccc(-c4ccc5c(c4)c4ccccc4n5-c4ccccc4)cc3)c3ccc(-c4cccc5c4ccc4ccccc45)cc3)cc2)cc1 ; c1ccc(-c2cccc(N(c3ccc(-c4ccc5c(c4)c4ccccc4n5-c4ccccc4)cc3)c3ccc(-c4cccc5c4ccc4ccccc45)cc3)c2)cc1 ; c1ccc(-c2ccccc2-c2ccccc2-c2cccc(N(c3ccc(-c4ccc5c(c4)c4ccccc4n5-c4ccccc4)cc3)c3ccc(-c4cccc5c4ccc4ccccc45)cc3)c2)cc1]

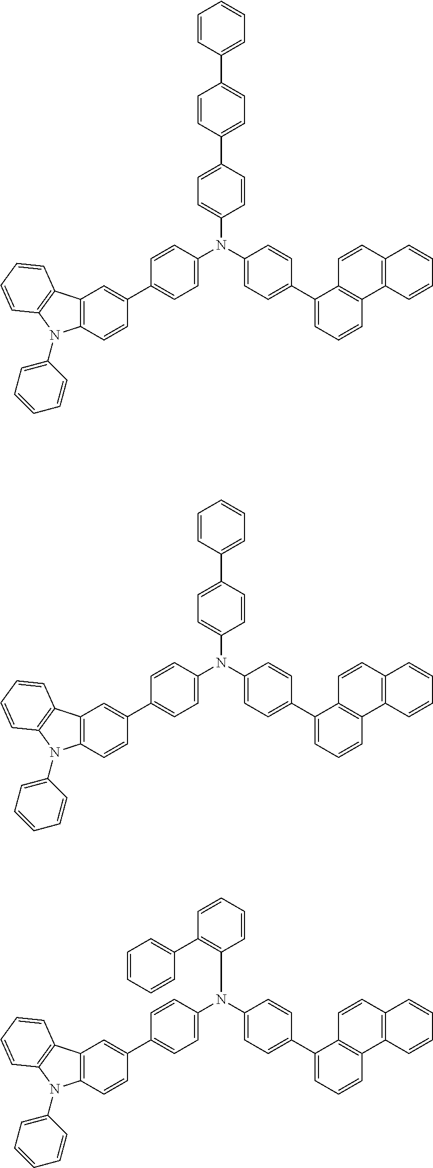 [SMILES: c1ccc(-c2ccc(-c3ccc(N(c4ccc(-c5ccc6c(c5)c5ccccc5n6-c5ccccc5)cc4)c4ccc(-c5cccc6c5ccc5ccccc56)cc4)cc3)cc2)cc1 ; c1ccc(-c2ccc(N(c3ccc(-c4ccc5c(c4)c4ccccc4n5-c4ccccc4)cc3)c3ccc(-c4cccc5c4ccc4ccccc45)cc3)cc2)cc1 ; c1ccc(-c2ccccc2N(c2ccc(-c3ccc4c(c3)c3ccccc3n4-c3ccccc3)cc2)c2ccc(-c3cccc4c3ccc3ccccc34)cc2)cc1]

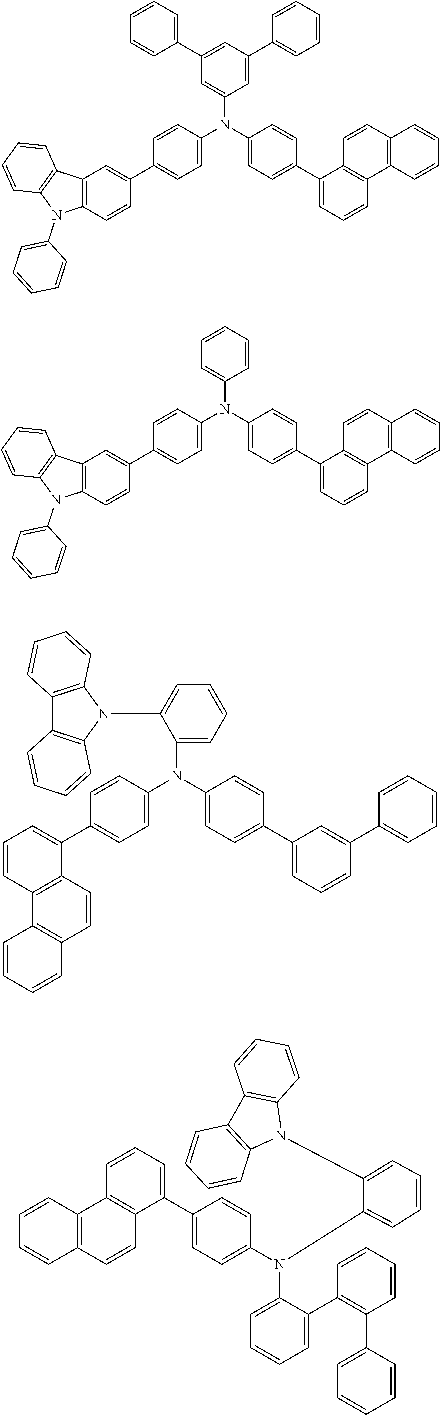 [SMILES: c1ccc(-c2cc(-c3ccccc3)cc(N(c3ccc(-c4ccc5c(c4)c4ccccc4n5-c4ccccc4)cc3)c3ccc(-c4cccc5c4ccc4ccccc45)cc3)c2)cc1 ; c1ccc(N(c2ccc(-c3ccc4c(c3)c3ccccc3n4-c3ccccc3)cc2)c2ccc(-c3cccc4c3ccc3ccccc34)cc2)cc1 ; c1ccc(-c2cccc(-c3ccc(N(c4ccc(-c5cccc6c5ccc5ccccc56)cc4)c4ccccc4-n4c5ccccc5c5ccccc54)cc3)c2)cc1 ; c1ccc(-c2ccccc2-c2ccccc2N(c2ccc(-c3cccc4c3ccc3ccccc34)cc2)c2ccccc2-n2c3ccccc3c3ccccc32)cc1]

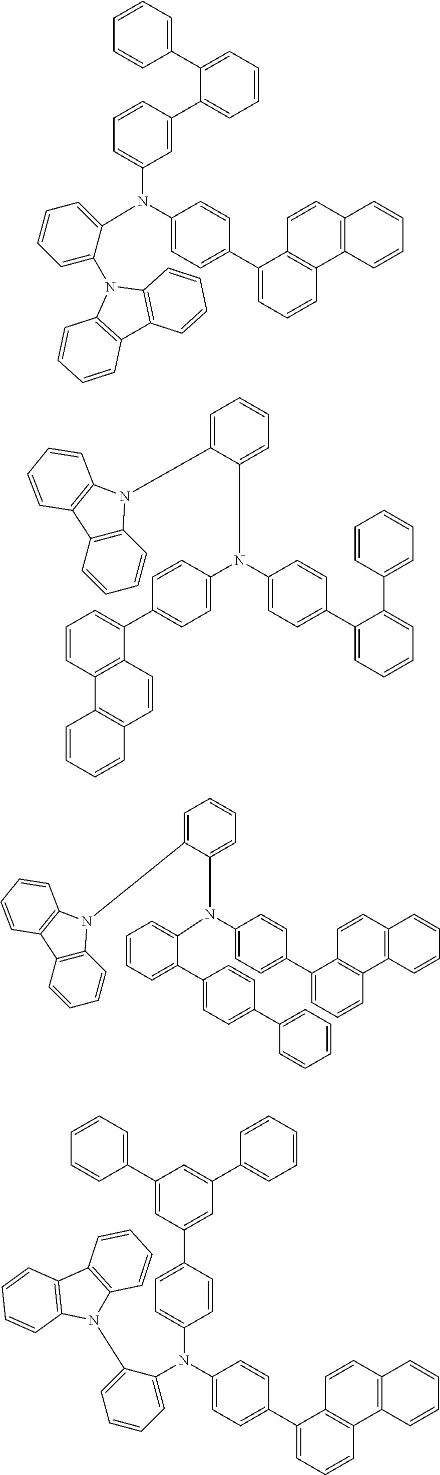 [SMILES: c1ccc(-c2ccccc2-c2cccc(N(c3ccc(-c4cccc5c4ccc4ccccc45)cc3)c3ccccc3-n3c4ccccc4c4ccccc43)c2)cc1 ; c1ccc(-c2ccccc2-c2ccc(N(c3ccc(-c4cccc5c4ccc4ccccc45)cc3)c3ccccc3-n3c4ccccc4c4ccccc43)cc2)cc1 ; c1ccc(-c2ccc(-c3ccccc3N(c3ccc(-c4cccc5c4ccc4ccccc45)cc3)c3ccccc3-n3c4ccccc4c4ccccc43)cc2)cc1 ; c1ccc(-c2cc(-c3ccccc3)cc(-c3ccc(N(c4ccc(-c5cccc6c5ccc5ccccc56)cc4)c4ccccc4-n4c5ccccc5c5ccccc54)cc3)c2)cc1]

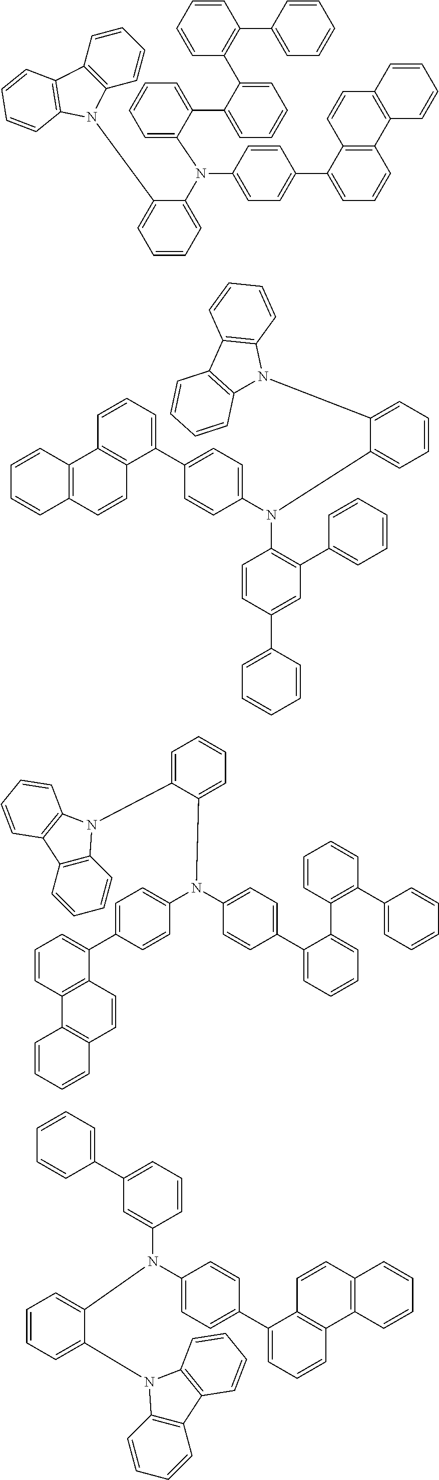

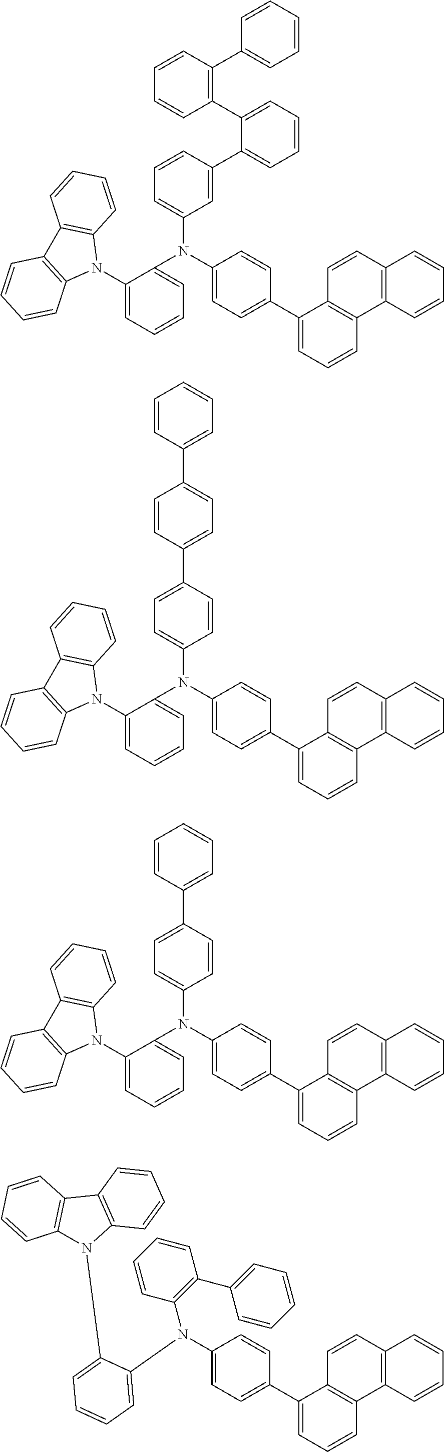 [SMILES: c1ccc(-c2ccccc2-c2ccccc2-c2cccc(N(c3ccc(-c4cccc5c4ccc4ccccc45)cc3)c3ccccc3-n3c4ccccc4c4ccccc43)c2)cc1 ; c1ccc(-c2ccc(-c3ccc(N(c4ccc(-c5cccc6c5ccc5ccccc56)cc4)c4ccccc4-n4c5ccccc5c5ccccc54)cc3)cc2)cc1 ; c1ccc(-c2ccc(N(c3ccc(-c4cccc5c4ccc4ccccc45)cc3)c3ccccc3-n3c4ccccc4c4ccccc43)cc2)cc1 ; c1ccc(-c2ccccc2N(c2ccc(-c3cccc4c3ccc3ccccc34)cc2)c2ccccc2-n2c3ccccc3c3ccccc32)cc1]

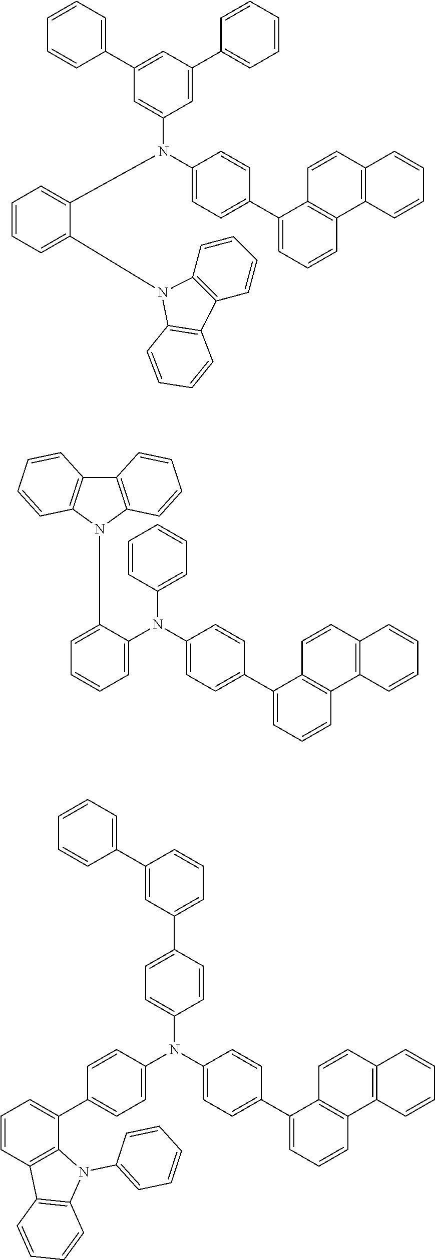 [SMILES: c1ccc(-c2cc(-c3ccccc3)cc(N(c3ccc(-c4cccc5c4ccc4ccccc45)cc3)c3ccccc3-n3c4ccccc4c4ccccc43)c2)cc1 ; c1ccc(N(c2ccc(-c3cccc4c3ccc3ccccc34)cc2)c2ccccc2-n2c3ccccc3c3ccccc32)cc1 ; c1ccc(-c2cccc(-c3ccc(N(c4ccc(-c5cccc6c5ccc5ccccc56)cc4)c4ccc(-c5cccc6c7ccccc7n(-c7ccccc7)c56)cc4)cc3)c2)cc1]

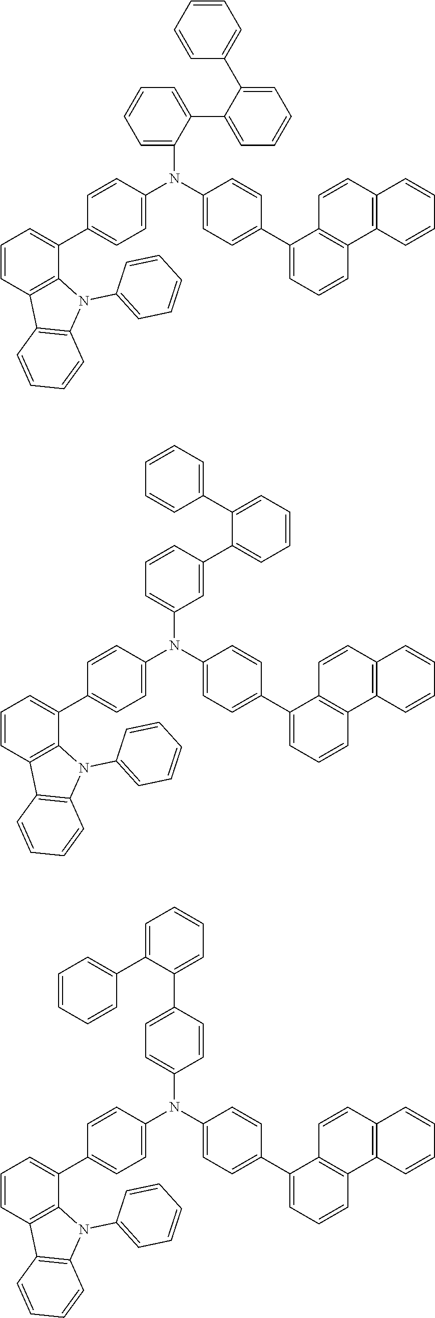 [SMILES: c1ccc(-c2ccccc2-c2ccccc2N(c2ccc(-c3cccc4c3ccc3ccccc34)cc2)c2ccc(-c3cccc4c5ccccc5n(-c5ccccc5)c34)cc2)cc1 ; c1ccc(-c2ccccc2-c2cccc(N(c3ccc(-c4cccc5c4ccc4ccccc45)cc3)c3ccc(-c4cccc5c6ccccc6n(-c6ccccc6)c45)cc3)c2)cc1 ; c1ccc(-c2ccccc2-c2ccc(N(c3ccc(-c4cccc5c4ccc4ccccc45)cc3)c3ccc(-c4cccc5c6ccccc6n(-c6ccccc6)c45)cc3)cc2)cc1]

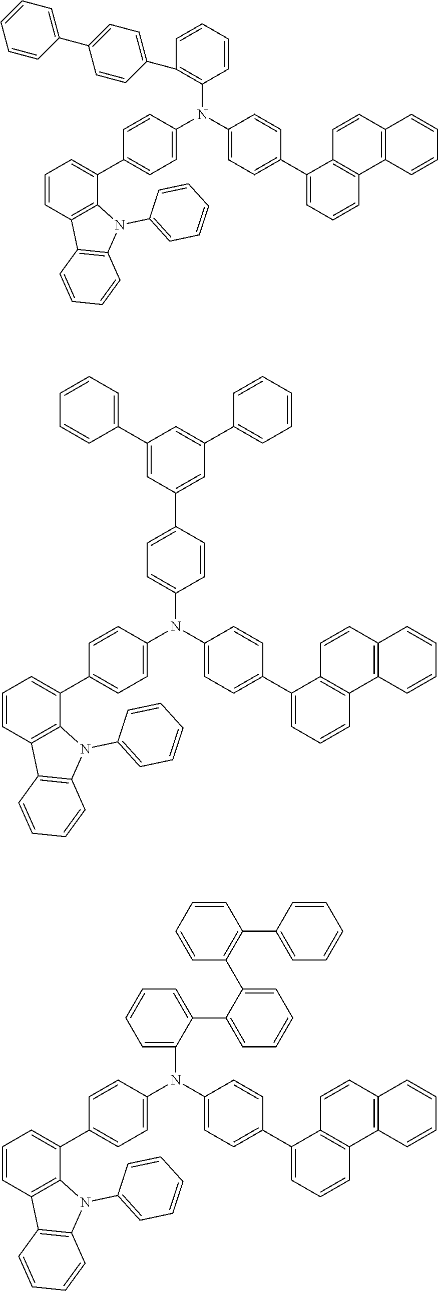 [SMILES: c1ccc(-c2ccc(-c3ccccc3N(c3ccc(-c4cccc5c4ccc4ccccc45)cc3)c3ccc(-c4cccc5c6ccccc6n(-c6ccccc6)c45)cc3)cc2)cc1 ; c1ccc(-c2cc(-c3ccccc3)cc(-c3ccc(N(c4ccc(-c5cccc6c5ccc5ccccc56)cc4)c4ccc(-c5cccc6c7ccccc7n(-c7ccccc7)c56)cc4)cc3)c2)cc1 ; c1ccc(-c2ccccc2-c2ccccc2-c2ccccc2N(c2ccc(-c3cccc4c3ccc3ccccc34)cc2)c2ccc(-c3cccc4c5ccccc5n(-c5ccccc5)c34)cc2)cc1]

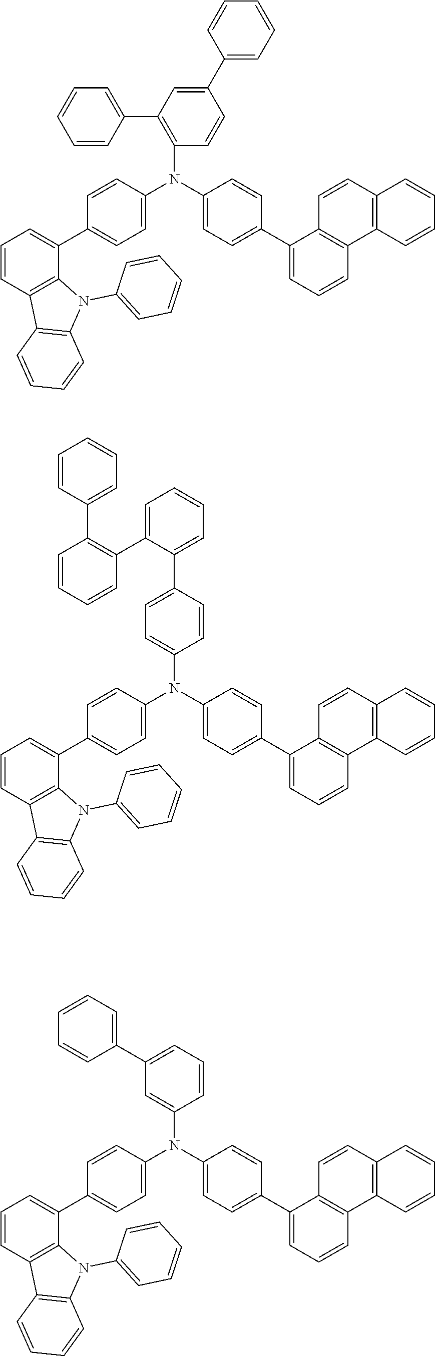 [SMILES: c1ccc(-c2ccc(N(c3ccc(-c4cccc5c4ccc4ccccc45)cc3)c3ccc(-c4cccc5c6ccccc6n(-c6ccccc6)c45)cc3)c(-c3ccccc3)c2)cc1 ; c1ccc(-c2ccccc2-c2ccccc2-c2ccc(N(c3ccc(-c4cccc5c4ccc4ccccc45)cc3)c3ccc(-c4cccc5c6ccccc6n(-c6ccccc6)c45)cc3)cc2)cc1 ; c1ccc(-c2cccc(N(c3ccc(-c4cccc5c4ccc4ccccc45)cc3)c3ccc(-c4cccc5c6ccccc6n(-c6ccccc6)c45)cc3)c2)cc1]

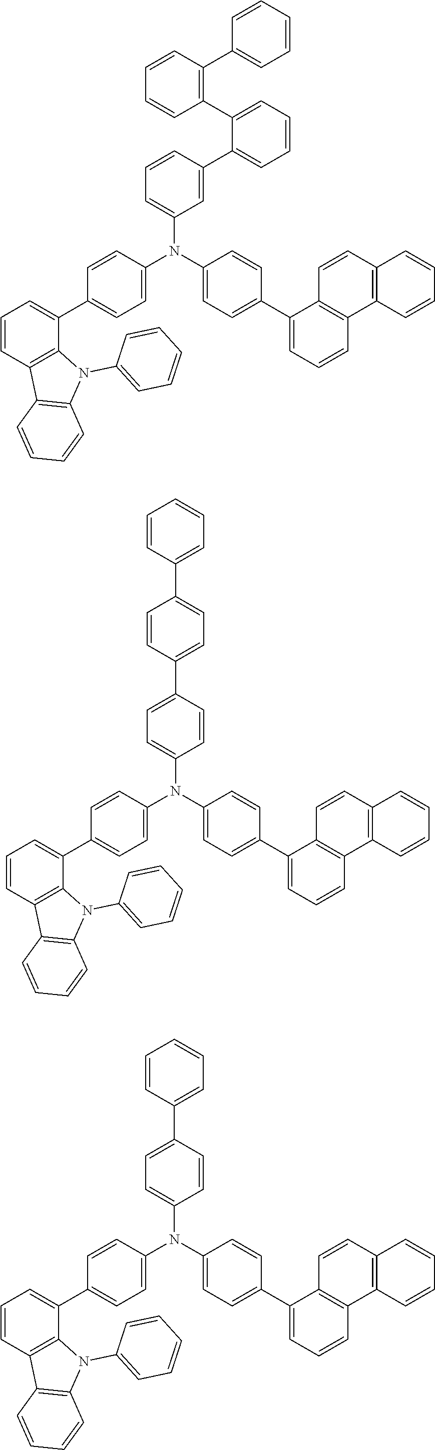 [SMILES: c1ccc(-c2ccccc2-c2ccccc2-c2cccc(N(c3ccc(-c4cccc5c4ccc4ccccc45)cc3)c3ccc(-c4cccc5c6ccccc6n(-c6ccccc6)c45)cc3)c2)cc1 ; c1ccc(-c2ccc(-c3ccc(N(c4ccc(-c5cccc6c5ccc5ccccc56)cc4)c4ccc(-c5cccc6c7ccccc7n(-c7ccccc7)c56)cc4)cc3)cc2)cc1 ; c1ccc(-c2ccc(N(c3ccc(-c4cccc5c4ccc4ccccc45)cc3)c3ccc(-c4cccc5c6ccccc6n(-c6ccccc6)c45)cc3)cc2)cc1]

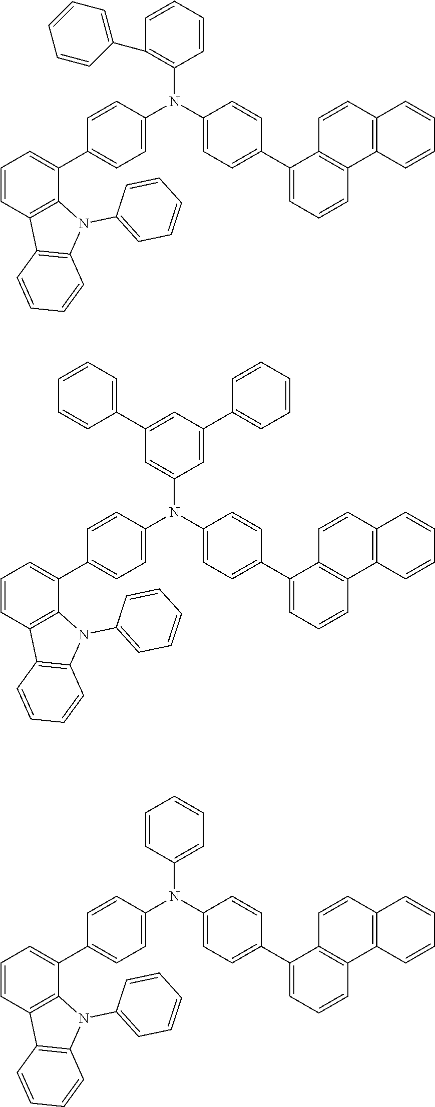 [SMILES: c1ccc(-c2ccccc2N(c2ccc(-c3cccc4c3ccc3ccccc34)cc2)c2ccc(-c3cccc4c5ccccc5n(-c5ccccc5)c34)cc2)cc1 ; c1ccc(-c2cc(-c3ccccc3)cc(N(c3ccc(-c4cccc5c4ccc4ccccc45)cc3)c3ccc(-c4cccc5c6ccccc6n(-c6ccccc6)c45)cc3)c2)cc1 ; c1ccc(N(c2ccc(-c3cccc4c3ccc3ccccc34)cc2)c2ccc(-c3cccc4c5ccccc5n(-c5ccccc5)c34)cc2)cc1]

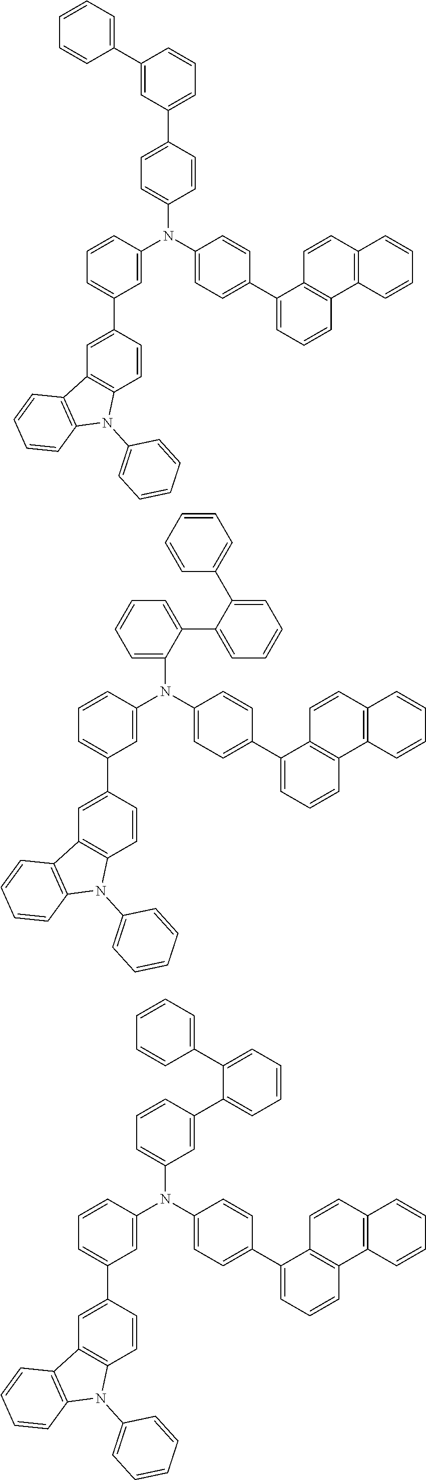

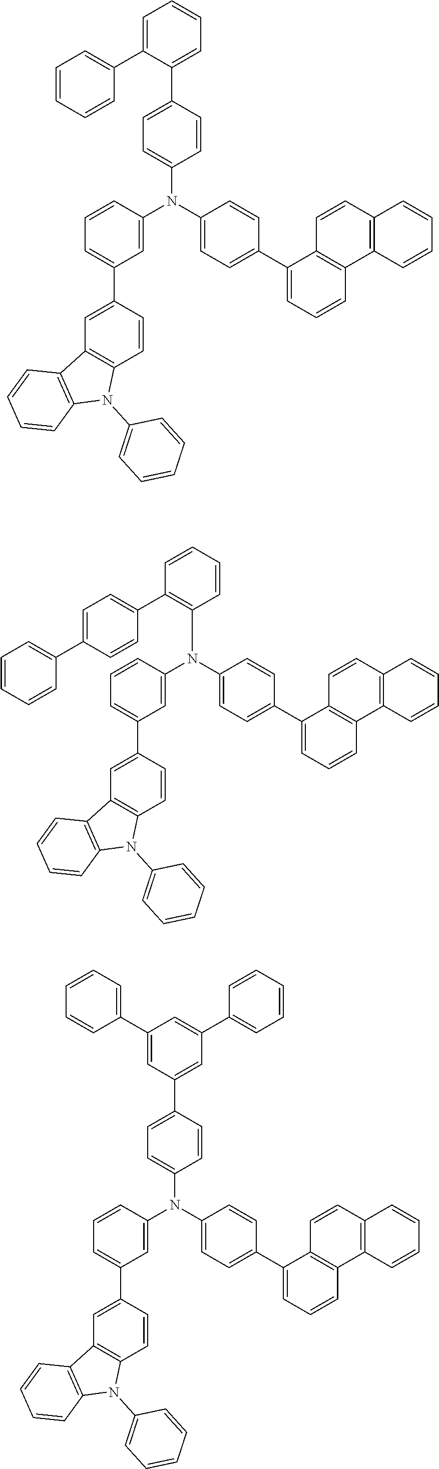 [SMILES: c1ccc(-c2ccccc2-c2ccc(N(c3ccc(-c4cccc5c4ccc4ccccc45)cc3)c3cccc(-c4ccc5c(c4)c4ccccc4n5-c4ccccc4)c3)cc2)cc1 ; c1ccc(-c2ccc(-c3ccccc3N(c3ccc(-c4cccc5c4ccc4ccccc45)cc3)c3cccc(-c4ccc5c(c4)c4ccccc4n5-c4ccccc4)c3)cc2)cc1 ; c1ccc(-c2cc(-c3ccccc3)cc(-c3ccc(N(c4ccc(-c5cccc6c5ccc5ccccc56)cc4)c4cccc(-c5ccc6c(c5)c5ccccc5n6-c5ccccc5)c4)cc3)c2)cc1]

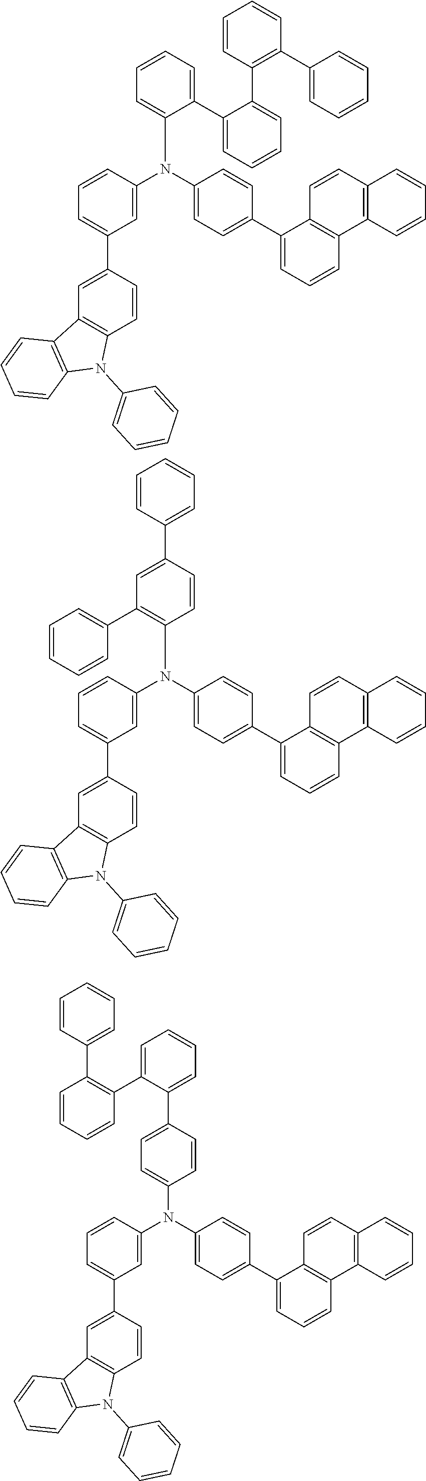

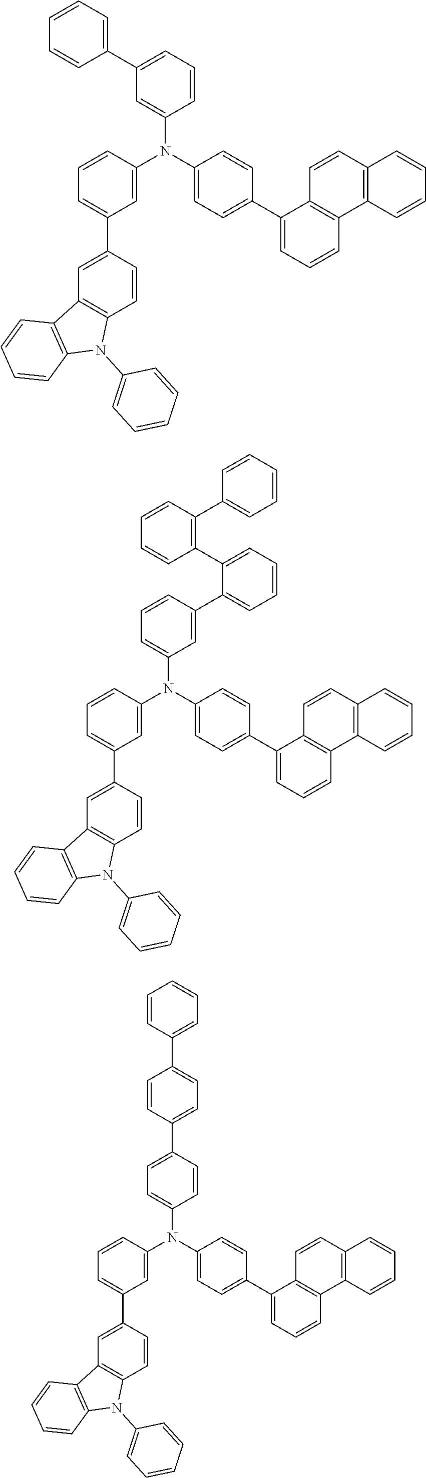

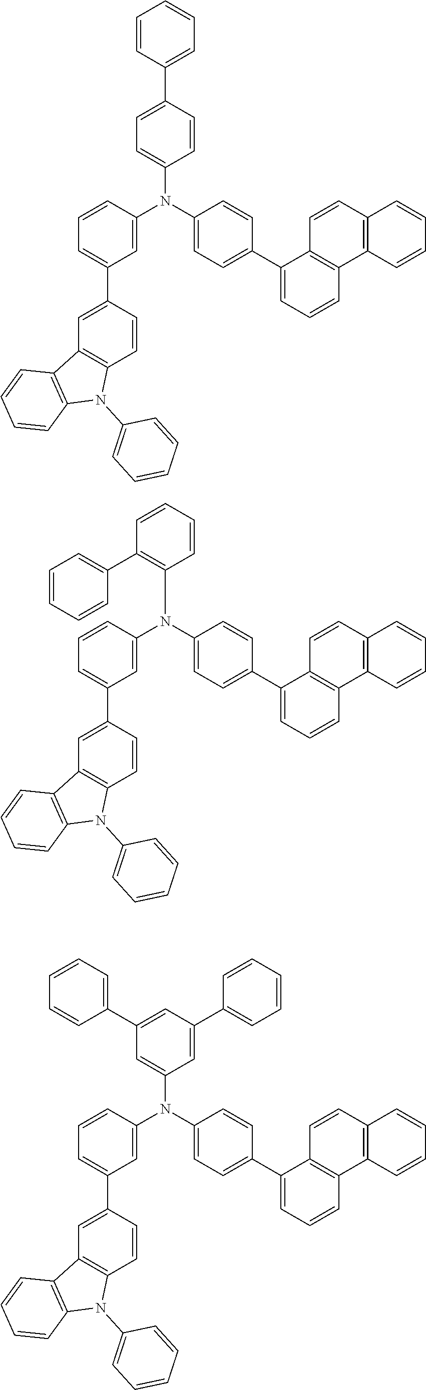

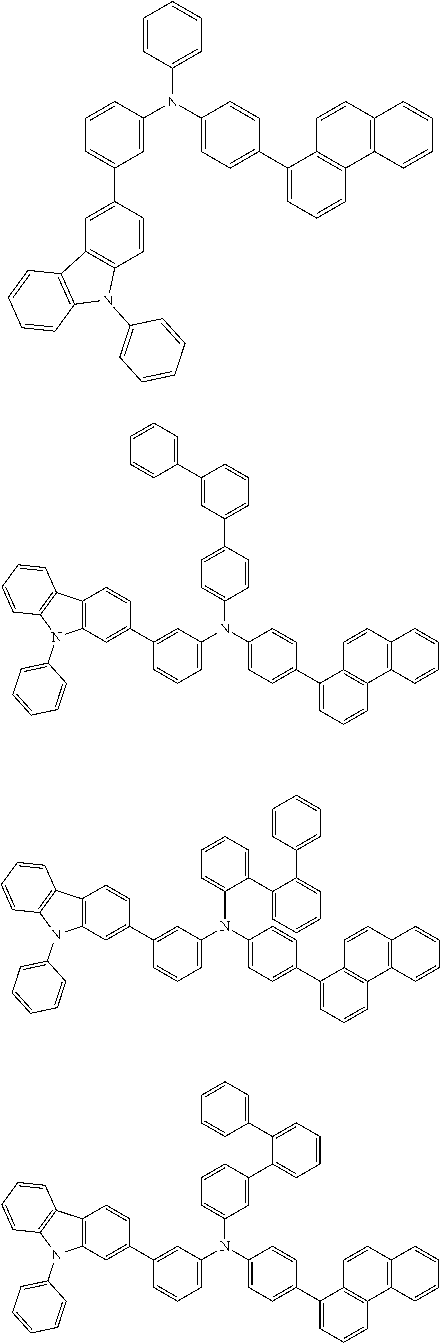 [SMILES: c1ccc(N(c2ccc(-c3cccc4c3ccc3ccccc34)cc2)c2cccc(-c3ccc4c(c3)c3ccccc3n4-c3ccccc3)c2)cc1 ; c1ccc(-c2cccc(-c3ccc(N(c4ccc(-c5cccc6c5ccc5ccccc56)cc4)c4cccc(-c5ccc6c7ccccc7n(-c7ccccc7)c6c5)c4)cc3)c2)cc1 ; c1ccc(-c2ccccc2-c2ccccc2N(c2ccc(-c3cccc4c3ccc3ccccc34)cc2)c2cccc(-c3ccc4c5ccccc5n(-c5ccccc5)c4c3)c2)cc1 ; c1ccc(-c2ccccc2-c2cccc(N(c3ccc(-c4cccc5c4ccc4ccccc45)cc3)c3cccc(-c4ccc5c6ccccc6n(-c6ccccc6)c5c4)c3)c2)cc1]

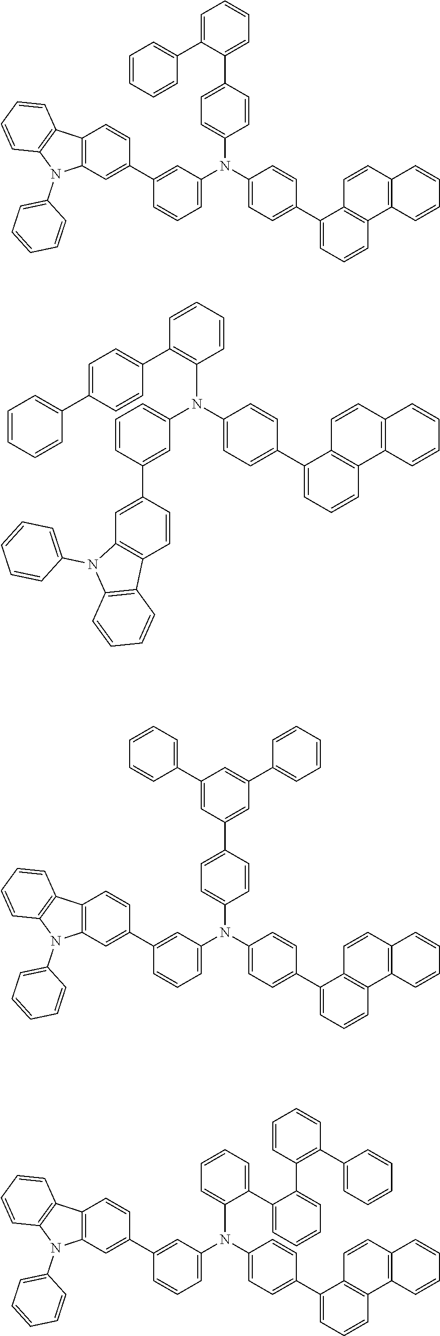 [SMILES: c1ccc(-c2ccccc2-c2ccc(N(c3ccc(-c4cccc5c4ccc4ccccc45)cc3)c3cccc(-c4ccc5c6ccccc6n(-c6ccccc6)c5c4)c3)cc2)cc1 ; c1ccc(-c2ccc(-c3ccccc3N(c3ccc(-c4cccc5c4ccc4ccccc45)cc3)c3cccc(-c4ccc5c6ccccc6n(-c6ccccc6)c5c4)c3)cc2)cc1 ; c1ccc(-c2cc(-c3ccccc3)cc(-c3ccc(N(c4ccc(-c5cccc6c5ccc5ccccc56)cc4)c4cccc(-c5ccc6c7ccccc7n(-c7ccccc7)c6c5)c4)cc3)c2)cc1 ; c1ccc(-c2ccccc2-c2ccccc2-c2ccccc2N(c2ccc(-c3cccc4c3ccc3ccccc34)cc2)c2cccc(-c3ccc4c5ccccc5n(-c5ccccc5)c4c3)c2)cc1]

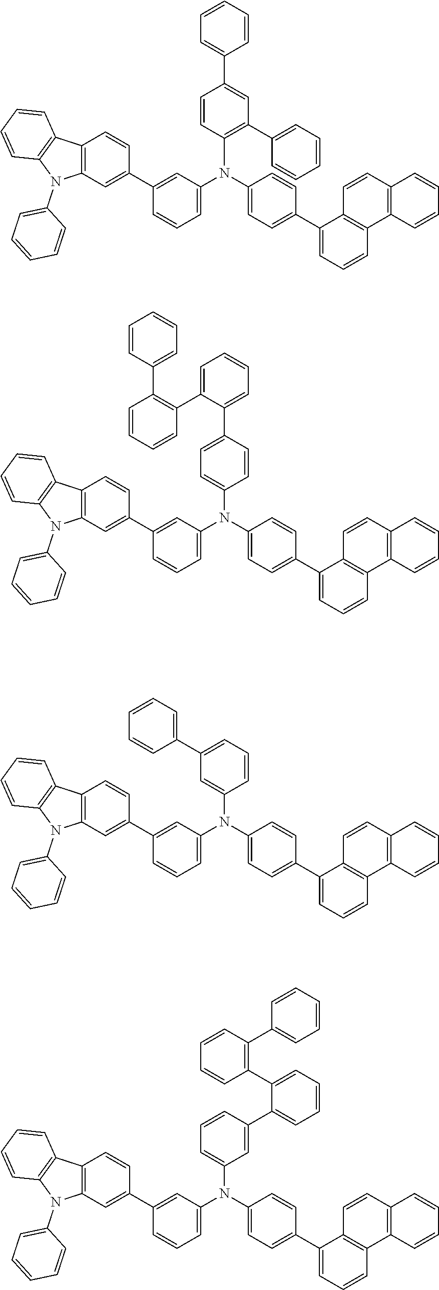 [SMILES: c1ccc(-c2ccc(N(c3ccc(-c4cccc5c4ccc4ccccc45)cc3)c3cccc(-c4ccc5c6ccccc6n(-c6ccccc6)c5c4)c3)c(-c3ccccc3)c2)cc1 ; c1ccc(-c2ccccc2-c2ccccc2-c2ccc(N(c3ccc(-c4cccc5c4ccc4ccccc45)cc3)c3cccc(-c4ccc5c6ccccc6n(-c6ccccc6)c5c4)c3)cc2)cc1 ; c1ccc(-c2cccc(N(c3ccc(-c4cccc5c4ccc4ccccc45)cc3)c3cccc(-c4ccc5c6ccccc6n(-c6ccccc6)c5c4)c3)c2)cc1 ; c1ccc(-c2ccccc2-c2ccccc2-c2cccc(N(c3ccc(-c4cccc5c4ccc4ccccc45)cc3)c3cccc(-c4ccc5c6ccccc6n(-c6ccccc6)c5c4)c3)c2)cc1]

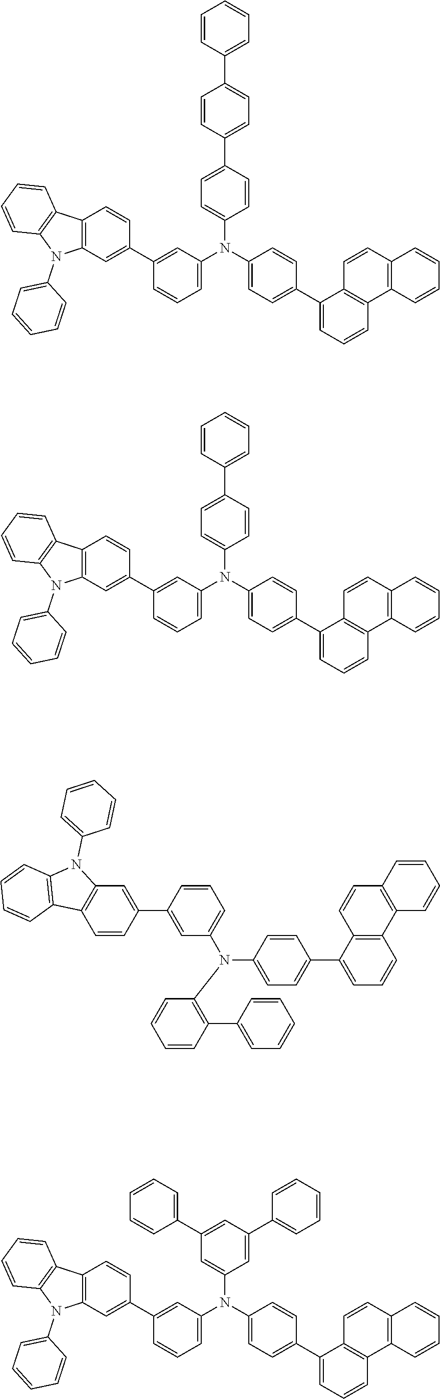 [SMILES: c1ccc(-c2ccc(-c3ccc(N(c4ccc(-c5cccc6c5ccc5ccccc56)cc4)c4cccc(-c5ccc6c7ccccc7n(-c7ccccc7)c6c5)c4)cc3)cc2)cc1 ; c1ccc(-c2ccc(N(c3ccc(-c4cccc5c4ccc4ccccc45)cc3)c3cccc(-c4ccc5c6ccccc6n(-c6ccccc6)c5c4)c3)cc2)cc1 ; c1ccc(-c2ccccc2N(c2ccc(-c3cccc4c3ccc3ccccc34)cc2)c2cccc(-c3ccc4c5ccccc5n(-c5ccccc5)c4c3)c2)cc1 ; c1ccc(-c2cc(-c3ccccc3)cc(N(c3ccc(-c4cccc5c4ccc4ccccc45)cc3)c3cccc(-c4ccc5c6ccccc6n(-c6ccccc6)c5c4)c3)c2)cc1]

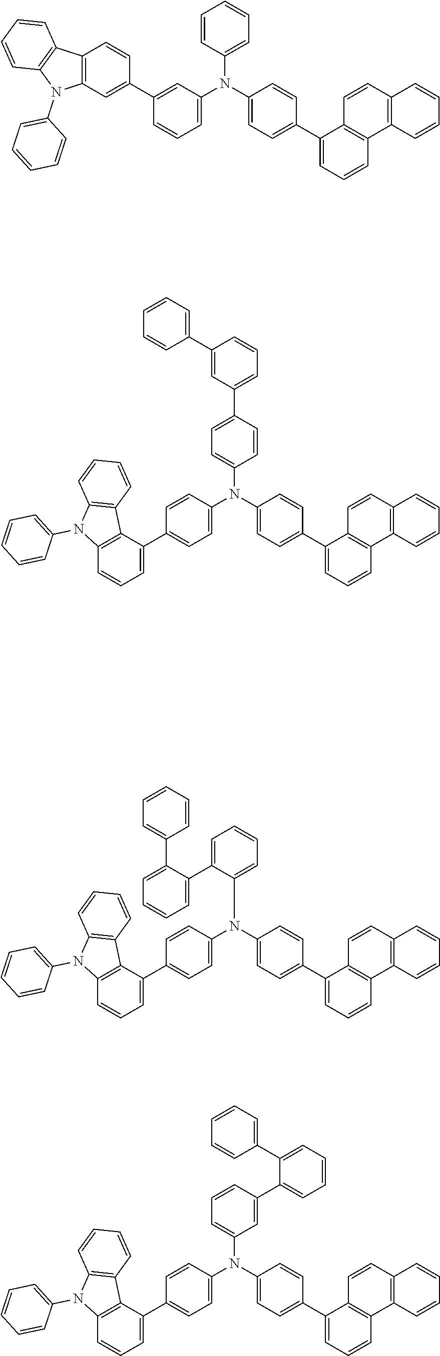 [SMILES: c1ccc(N(c2ccc(-c3cccc4c3ccc3ccccc34)cc2)c2cccc(-c3ccc4c5ccccc5n(-c5ccccc5)c4c3)c2)cc1 ; c1ccc(-c2cccc(-c3ccc(N(c4ccc(-c5cccc6c5ccc5ccccc56)cc4)c4ccc(-c5cccc6c5c5ccccc5n6-c5ccccc5)cc4)cc3)c2)cc1 ; c1ccc(-c2ccccc2-c2ccccc2N(c2ccc(-c3cccc4c3ccc3ccccc34)cc2)c2ccc(-c3cccc4c3c3ccccc3n4-c3ccccc3)cc2)cc1 ; c1ccc(-c2ccccc2-c2cccc(N(c3ccc(-c4cccc5c4ccc4ccccc45)cc3)c3ccc(-c4cccc5c4c4ccccc4n5-c4ccccc4)cc3)c2)cc1]

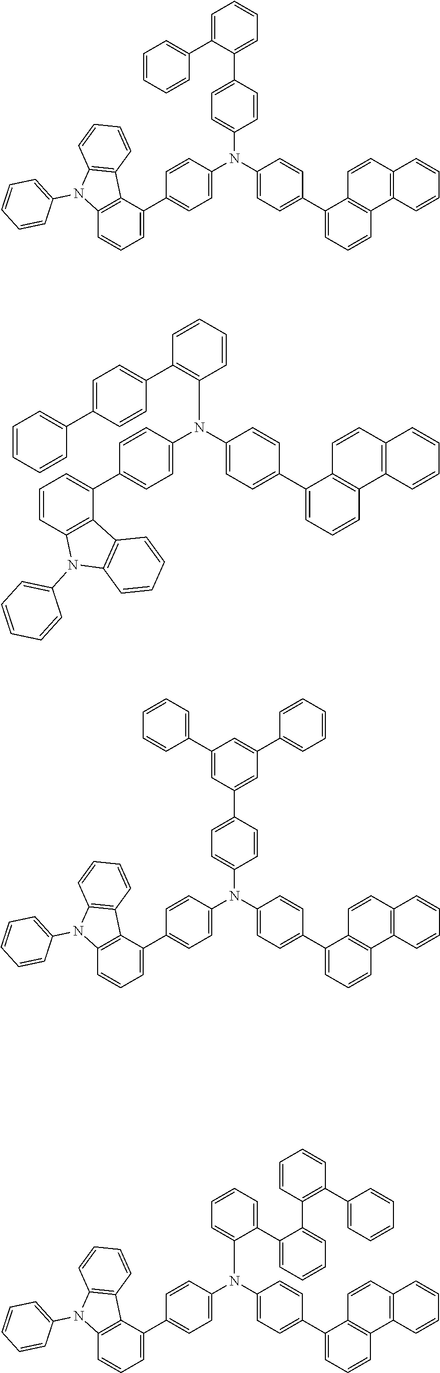 [SMILES: c1ccc(-c2ccccc2-c2ccc(N(c3ccc(-c4cccc5c4ccc4ccccc45)cc3)c3ccc(-c4cccc5c4c4ccccc4n5-c4ccccc4)cc3)cc2)cc1 ; c1ccc(-c2ccc(-c3ccccc3N(c3ccc(-c4cccc5c4ccc4ccccc45)cc3)c3ccc(-c4cccc5c4c4ccccc4n5-c4ccccc4)cc3)cc2)cc1 ; c1ccc(-c2cc(-c3ccccc3)cc(-c3ccc(N(c4ccc(-c5cccc6c5ccc5ccccc56)cc4)c4ccc(-c5cccc6c5c5ccccc5n6-c5ccccc5)cc4)cc3)c2)cc1 ; c1ccc(-c2ccccc2-c2ccccc2-c2ccccc2N(c2ccc(-c3cccc4c3ccc3ccccc34)cc2)c2ccc(-c3cccc4c3c3ccccc3n4-c3ccccc3)cc2)cc1]

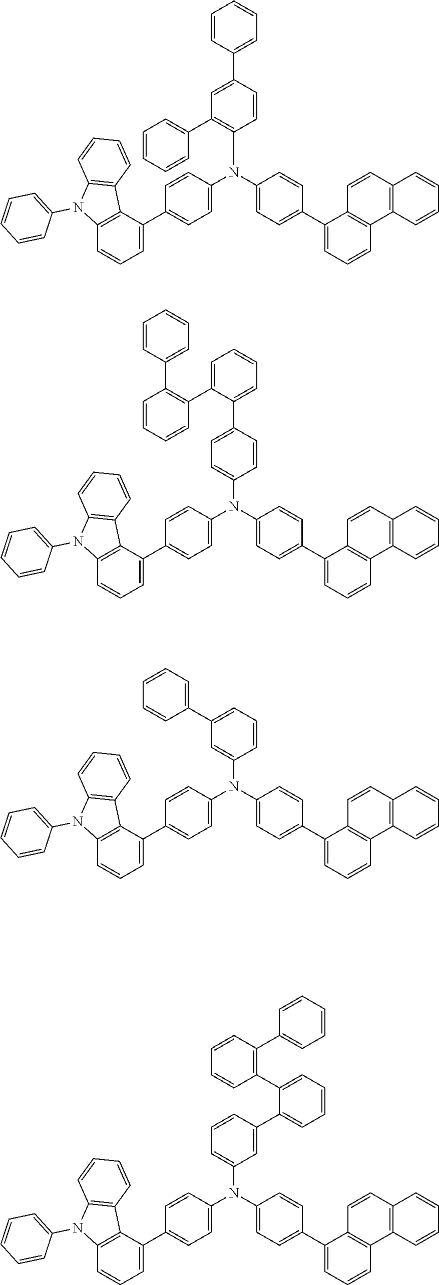 [SMILES: c1ccc(-c2ccc(N(c3ccc(-c4cccc5c4ccc4ccccc45)cc3)c3ccc(-c4cccc5c4c4ccccc4n5-c4ccccc4)cc3)c(-c3ccccc3)c2)cc1 ; c1ccc(-c2ccccc2-c2ccccc2-c2ccc(N(c3ccc(-c4cccc5c4ccc4ccccc45)cc3)c3ccc(-c4cccc5c4c4ccccc4n5-c4ccccc4)cc3)cc2)cc1 ; c1ccc(-c2cccc(N(c3ccc(-c4cccc5c4ccc4ccccc45)cc3)c3ccc(-c4cccc5c4c4ccccc4n5-c4ccccc4)cc3)c2)cc1 ; c1ccc(-c2ccccc2-c2ccccc2-c2cccc(N(c3ccc(-c4cccc5c4ccc4ccccc45)cc3)c3ccc(-c4cccc5c4c4ccccc4n5-c4ccccc4)cc3)c2)cc1]

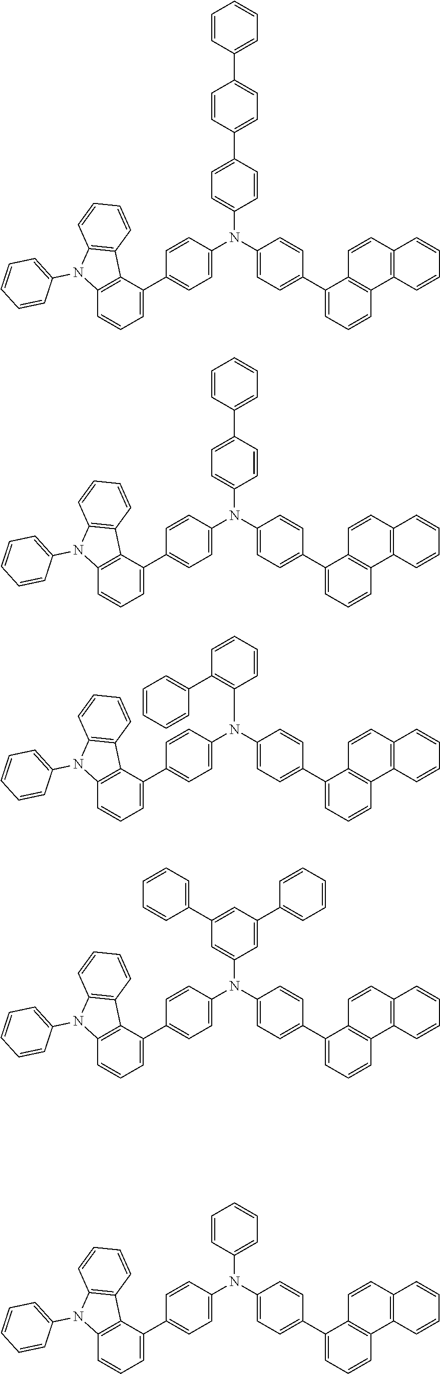 [SMILES: c1ccc(-c2ccc(-c3ccc(N(c4ccc(-c5cccc6c5ccc5ccccc56)cc4)c4ccc(-c5cccc6c5c5ccccc5n6-c5ccccc5)cc4)cc3)cc2)cc1 ; c1ccc(-c2ccc(N(c3ccc(-c4cccc5c4ccc4ccccc45)cc3)c3ccc(-c4cccc5c4c4ccccc4n5-c4ccccc4)cc3)cc2)cc1 ; c1ccc(-c2ccccc2N(c2ccc(-c3cccc4c3ccc3ccccc34)cc2)c2ccc(-c3cccc4c3c3ccccc3n4-c3ccccc3)cc2)cc1 ; c1ccc(-c2cc(-c3ccccc3)cc(N(c3ccc(-c4cccc5c4ccc4ccccc45)cc3)c3ccc(-c4cccc5c4c4ccccc4n5-c4ccccc4)cc3)c2)cc1 ; c1ccc(N(c2ccc(-c3cccc4c3ccc3ccccc34)cc2)c2ccc(-c3cccc4c3c3ccccc3n4-c3ccccc3)cc2)cc1]

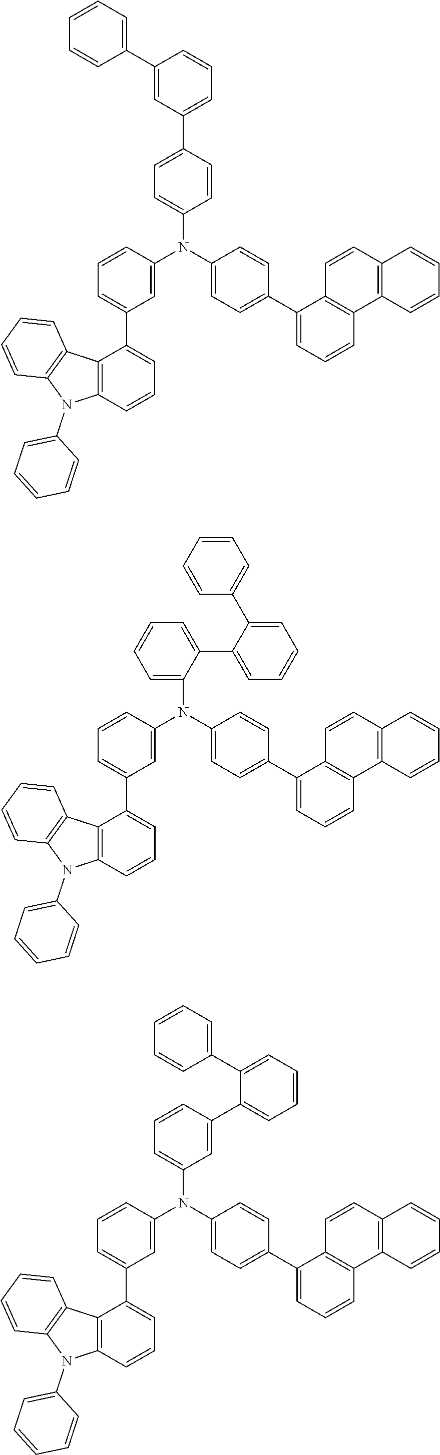 [SMILES: c1ccc(-c2cccc(-c3ccc(N(c4ccc(-c5cccc6c5ccc5ccccc56)cc4)c4cccc(-c5cccc6c5c5ccccc5n6-c5ccccc5)c4)cc3)c2)cc1 ; c1ccc(-c2ccccc2-c2ccccc2N(c2ccc(-c3cccc4c3ccc3ccccc34)cc2)c2cccc(-c3cccc4c3c3ccccc3n4-c3ccccc3)c2)cc1 ; c1ccc(-c2ccccc2-c2cccc(N(c3ccc(-c4cccc5c4ccc4ccccc45)cc3)c3cccc(-c4cccc5c4c4ccccc4n5-c4ccccc4)c3)c2)cc1]

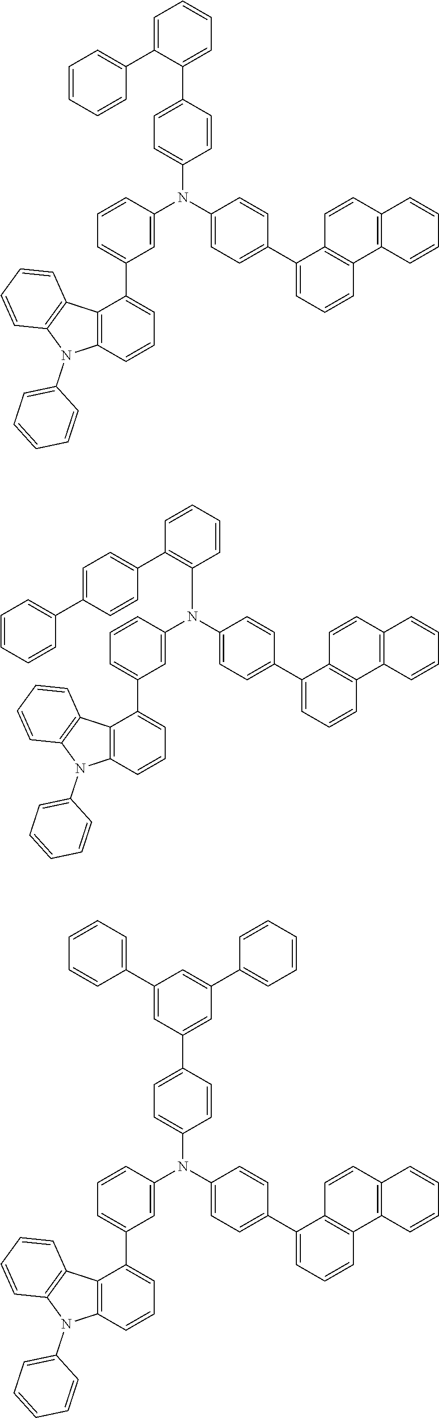 [SMILES: c1ccc(-c2ccccc2-c2ccc(N(c3ccc(-c4cccc5c4ccc4ccccc45)cc3)c3cccc(-c4cccc5c4c4ccccc4n5-c4ccccc4)c3)cc2)cc1 ; c1ccc(-c2ccc(-c3ccccc3N(c3ccc(-c4cccc5c4ccc4ccccc45)cc3)c3cccc(-c4cccc5c4c4ccccc4n5-c4ccccc4)c3)cc2)cc1 ; c1ccc(-c2cc(-c3ccccc3)cc(-c3ccc(N(c4ccc(-c5cccc6c5ccc5ccccc56)cc4)c4cccc(-c5cccc6c5c5ccccc5n6-c5ccccc5)c4)cc3)c2)cc1]

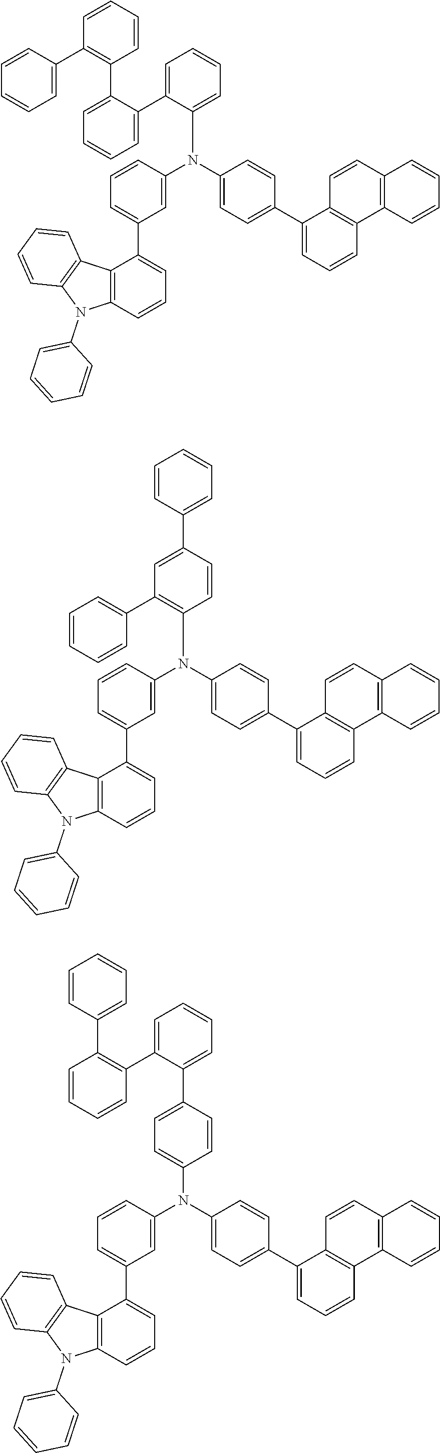 [SMILES: c1ccc(-c2ccccc2-c2ccccc2-c2ccccc2N(c2ccc(-c3cccc4c3ccc3ccccc34)cc2)c2cccc(-c3cccc4c3c3ccccc3n4-c3ccccc3)c2)cc1 ; c1ccc(-c2ccc(N(c3ccc(-c4cccc5c4ccc4ccccc45)cc3)c3cccc(-c4cccc5c4c4ccccc4n5-c4ccccc4)c3)c(-c3ccccc3)c2)cc1 ; c1ccc(-c2ccccc2-c2ccccc2-c2ccc(N(c3ccc(-c4cccc5c4ccc4ccccc45)cc3)c3cccc(-c4cccc5c4c4ccccc4n5-c4ccccc4)c3)cc2)cc1]

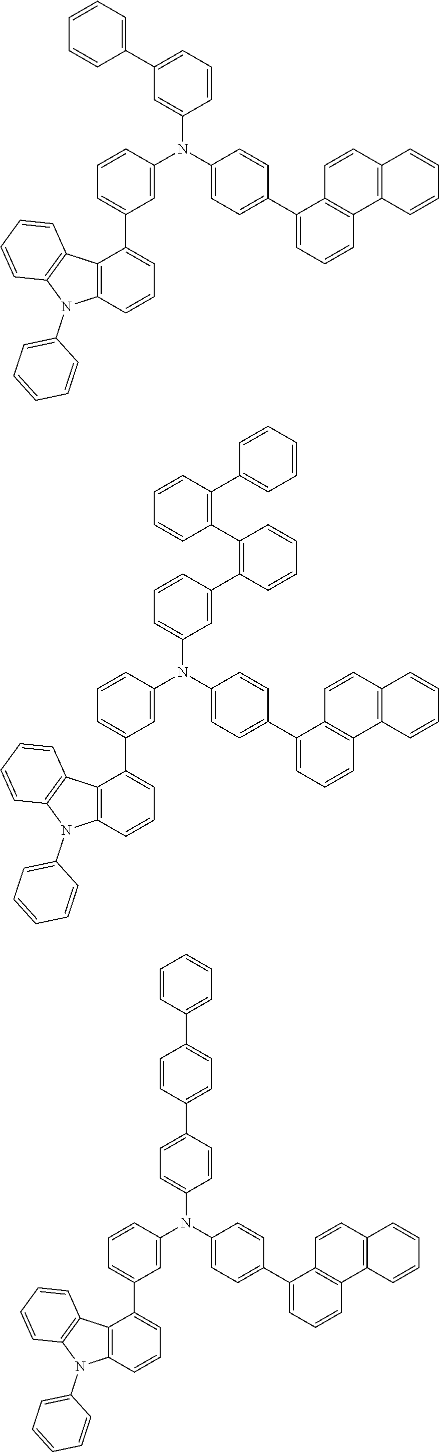 [SMILES: c1ccc(-c2cccc(N(c3ccc(-c4cccc5c4ccc4ccccc45)cc3)c3cccc(-c4cccc5c4c4ccccc4n5-c4ccccc4)c3)c2)cc1 ; c1ccc(-c2ccccc2-c2ccccc2-c2cccc(N(c3ccc(-c4cccc5c4ccc4ccccc45)cc3)c3cccc(-c4cccc5c4c4ccccc4n5-c4ccccc4)c3)c2)cc1 ; c1ccc(-c2ccc(-c3ccc(N(c4ccc(-c5cccc6c5ccc5ccccc56)cc4)c4cccc(-c5cccc6c5c5ccccc5n6-c5ccccc5)c4)cc3)cc2)cc1]

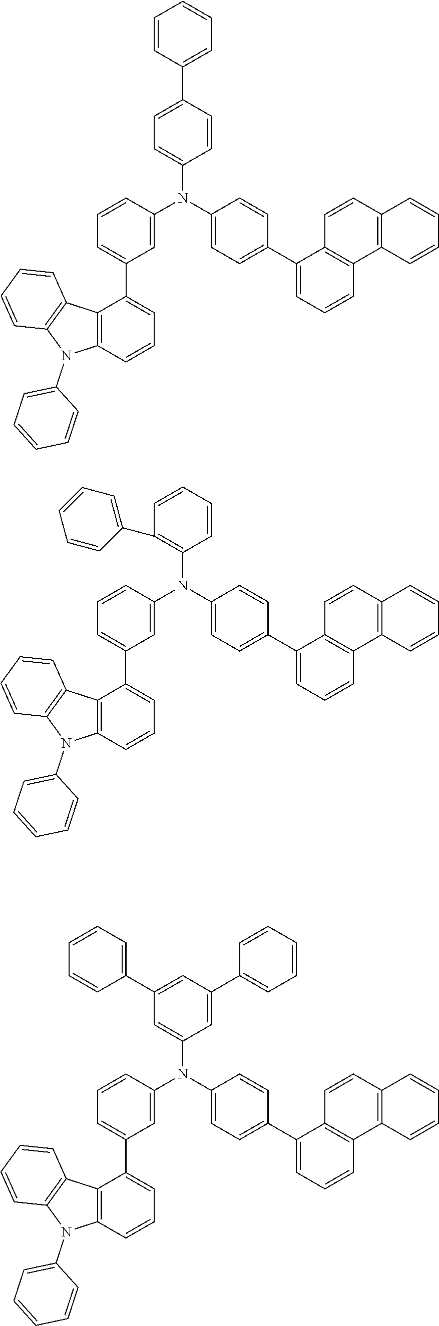 [SMILES: c1ccc(-c2ccc(N(c3ccc(-c4cccc5c4ccc4ccccc45)cc3)c3cccc(-c4cccc5c4c4ccccc4n5-c4ccccc4)c3)cc2)cc1 ; c1ccc(-c2ccccc2N(c2ccc(-c3cccc4c3ccc3ccccc34)cc2)c2cccc(-c3cccc4c3c3ccccc3n4-c3ccccc3)c2)cc1 ; c1ccc(-c2cc(-c3ccccc3)cc(N(c3ccc(-c4cccc5c4ccc4ccccc45)cc3)c3cccc(-c4cccc5c4c4ccccc4n5-c4ccccc4)c3)c2)cc1]

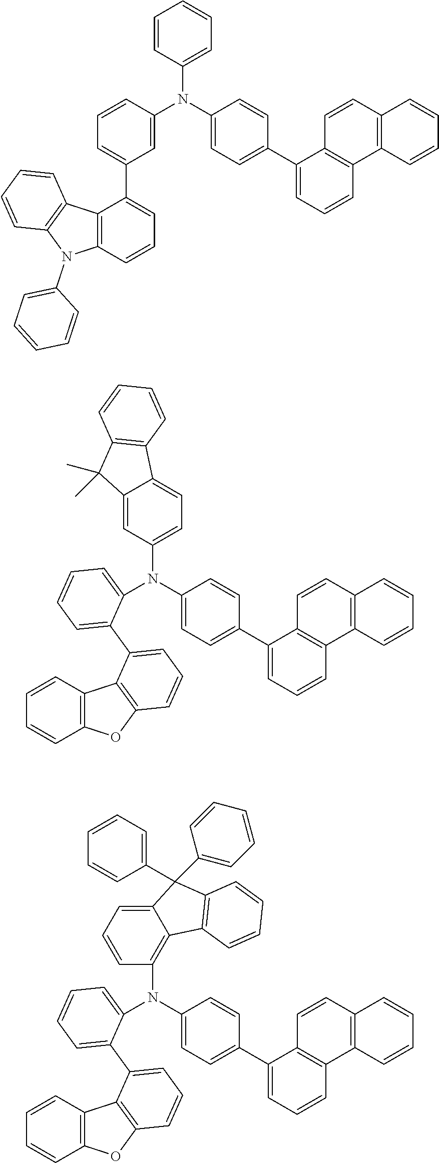 [SMILES: c1ccc(N(c2ccc(-c3cccc4c3ccc3ccccc34)cc2)c2cccc(-c3cccc4c3c3ccccc3n4-c3ccccc3)c2)cc1 ; CC1(C)c2ccccc2-c2ccc(N(c3ccc(-c4cccc5c4ccc4ccccc45)cc3)c3ccccc3-c3cccc4oc5ccccc5c34)cc21 ; c1ccc(C2(c3ccccc3)c3ccccc3-c3c(N(c4ccc(-c5cccc6c5ccc5ccccc56)cc4)c4ccccc4-c4cccc5oc6ccccc6c45)cccc32)cc1]

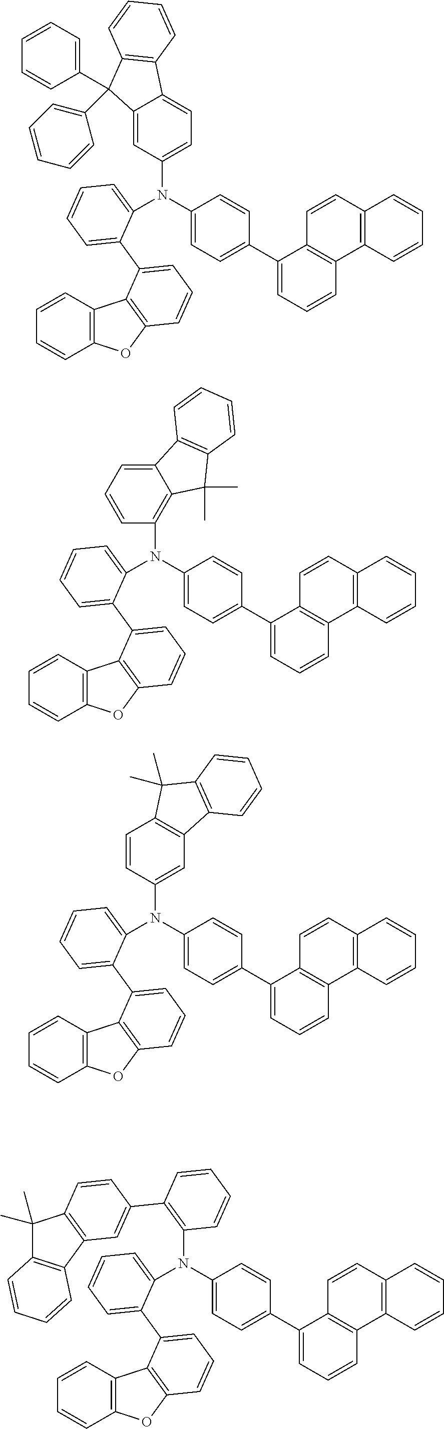 [SMILES: c1ccc(C2(c3ccccc3)c3ccccc3-c3ccc(N(c4ccc(-c5cccc6c5ccc5ccccc56)cc4)c4ccccc4-c4cccc5oc6ccccc6c45)cc32)cc1 ; CC1(C)c2ccccc2-c2cccc(N(c3ccc(-c4cccc5c4ccc4ccccc45)cc3)c3ccccc3-c3cccc4oc5ccccc5c34)c21 ; CC1(C)c2ccccc2-c2cc(N(c3ccc(-c4cccc5c4ccc4ccccc45)cc3)c3ccccc3-c3cccc4oc5ccccc5c34)ccc21 ; CC1(C)c2ccccc2-c2cc(-c3ccccc3N(c3ccc(-c4cccc5c4ccc4ccccc45)cc3)c3ccccc3-c3cccc4oc5ccccc5c34)ccc21]

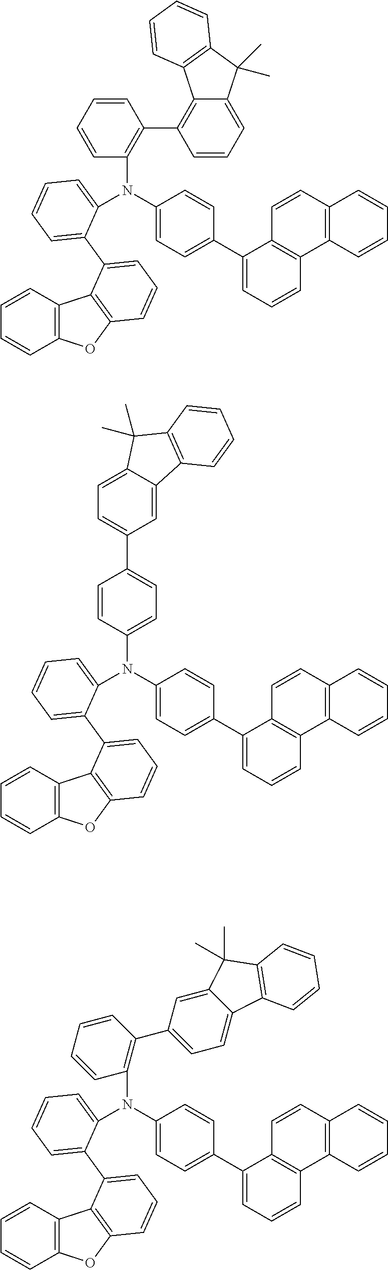 [SMILES: CC1(C)c2ccccc2-c2c(-c3ccccc3N(c3ccc(-c4cccc5c4ccc4ccccc45)cc3)c3ccccc3-c3cccc4oc5ccccc5c34)cccc21 ; CC1(C)c2ccccc2-c2cc(-c3ccc(N(c4ccc(-c5cccc6c5ccc5ccccc56)cc4)c4ccccc4-c4cccc5oc6ccccc6c45)cc3)ccc21 ; CC1(C)c2ccccc2-c2ccc(-c3ccccc3N(c3ccc(-c4cccc5c4ccc4ccccc45)cc3)c3ccccc3-c3cccc4oc5ccccc5c34)cc21]

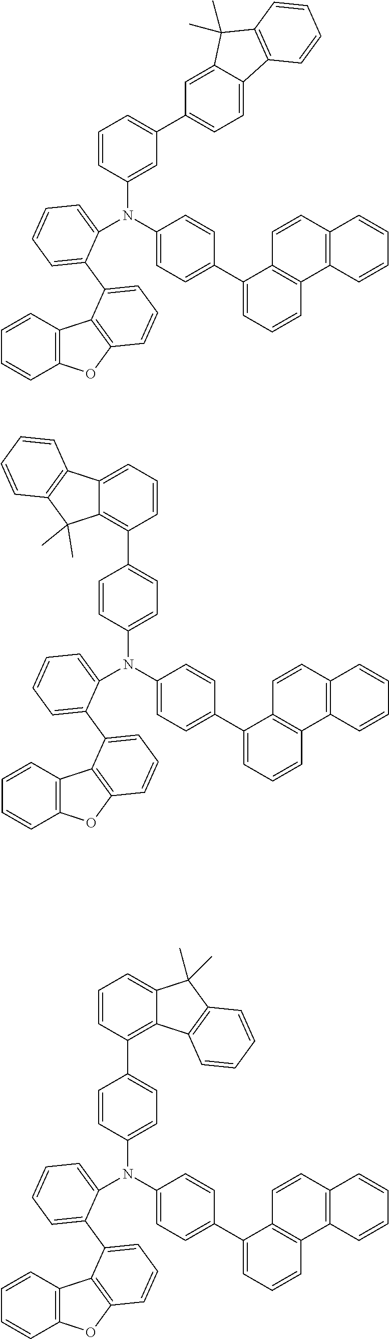 [SMILES: CC1(C)c2ccccc2-c2ccc(-c3cccc(N(c4ccc(-c5cccc6c5ccc5ccccc56)cc4)c4ccccc4-c4cccc5oc6ccccc6c45)c3)cc21 ; CC1(C)c2ccccc2-c2cccc(-c3ccc(N(c4ccc(-c5cccc6c5ccc5ccccc56)cc4)c4ccccc4-c4cccc5oc6ccccc6c45)cc3)c21 ; CC1(C)c2ccccc2-c2c(-c3ccc(N(c4ccc(-c5cccc6c5ccc5ccccc56)cc4)c4ccccc4-c4cccc5oc6ccccc6c45)cc3)cccc21]

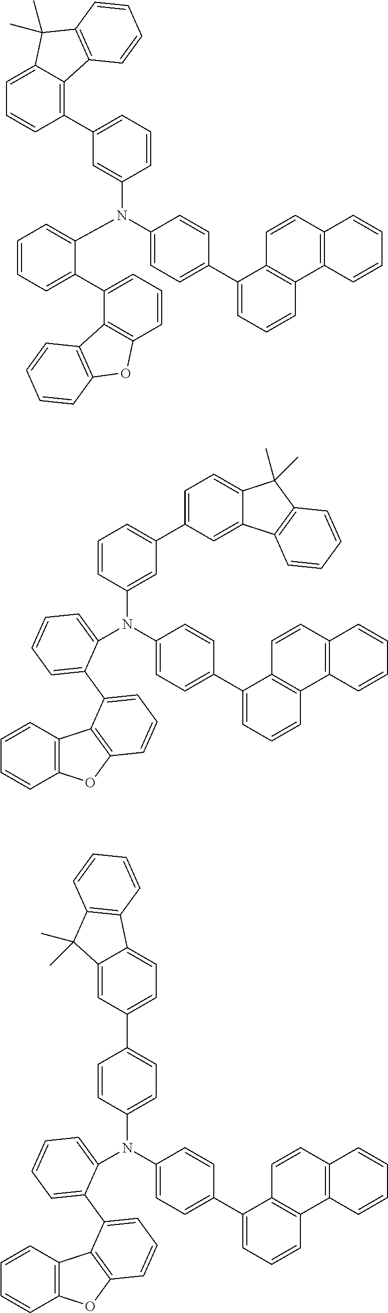 [SMILES: CC1(C)c2ccccc2-c2c(-c3cccc(N(c4ccc(-c5cccc6c5ccc5ccccc56)cc4)c4ccccc4-c4cccc5oc6ccccc6c45)c3)cccc21 ; CC1(C)c2ccccc2-c2cc(-c3cccc(N(c4ccc(-c5cccc6c5ccc5ccccc56)cc4)c4ccccc4-c4cccc5oc6ccccc6c45)c3)ccc21 ; CC1(C)c2ccccc2-c2ccc(-c3ccc(N(c4ccc(-c5cccc6c5ccc5ccccc56)cc4)c4ccccc4-c4cccc5oc6ccccc6c45)cc3)cc21]

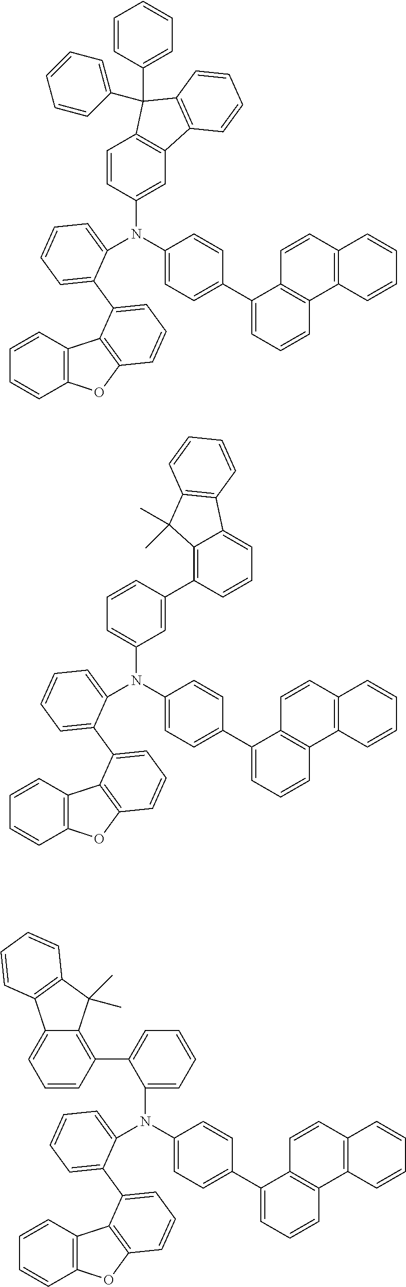 [SMILES: c1ccc(C2(c3ccccc3)c3ccccc3-c3cc(N(c4ccc(-c5cccc6c5ccc5ccccc56)cc4)c4ccccc4-c4cccc5oc6ccccc6c45)ccc32)cc1 ; CC1(C)c2ccccc2-c2cccc(-c3cccc(N(c4ccc(-c5cccc6c5ccc5ccccc56)cc4)c4ccccc4-c4cccc5oc6ccccc6c45)c3)c21 ; CC1(C)c2ccccc2-c2cccc(-c3ccccc3N(c3ccc(-c4cccc5c4ccc4ccccc45)cc3)c3ccccc3-c3cccc4oc5ccccc5c34)c21]

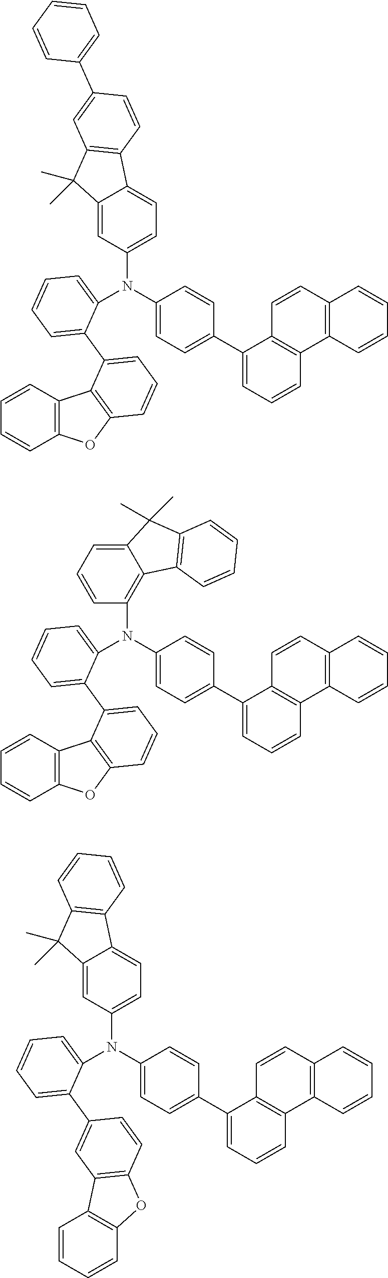 [SMILES: CC1(C)c2cc(-c3ccccc3)ccc2-c2ccc(N(c3ccc(-c4cccc5c4ccc4ccccc45)cc3)c3ccccc3-c3cccc4oc5ccccc5c34)cc21 ; CC1(C)c2ccccc2-c2c(N(c3ccc(-c4cccc5c4ccc4ccccc45)cc3)c3ccccc3-c3cccc4oc5ccccc5c34)cccc21 ; CC1(C)c2ccccc2-c2ccc(N(c3ccc(-c4cccc5c4ccc4ccccc45)cc3)c3ccccc3-c3ccc4oc5ccccc5c4c3)cc21]

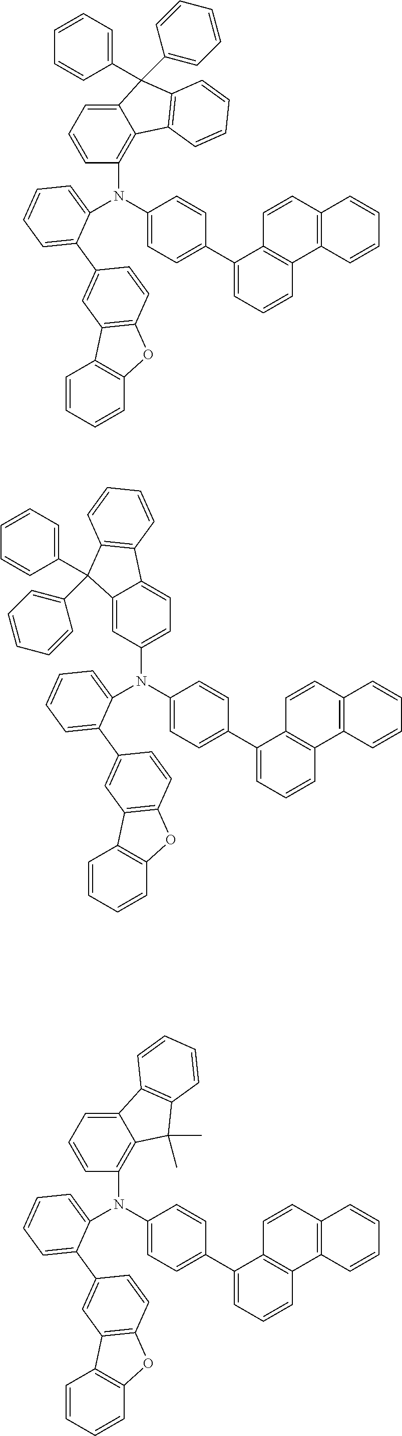 [SMILES: c1ccc(C2(c3ccccc3)c3ccccc3-c3c(N(c4ccc(-c5cccc6c5ccc5ccccc56)cc4)c4ccccc4-c4ccc5oc6ccccc6c5c4)cccc32)cc1 ; c1ccc(C2(c3ccccc3)c3ccccc3-c3ccc(N(c4ccc(-c5cccc6c5ccc5ccccc56)cc4)c4ccccc4-c4ccc5oc6ccccc6c5c4)cc32)cc1 ; CC1(C)c2ccccc2-c2cccc(N(c3ccc(-c4cccc5c4ccc4ccccc45)cc3)c3ccccc3-c3ccc4oc5ccccc5c4c3)c21]

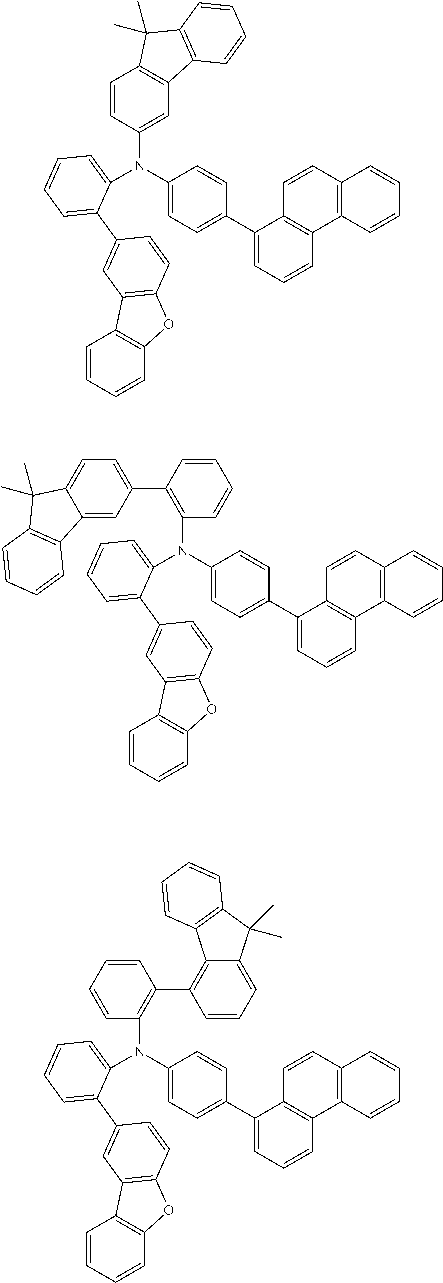 [SMILES: CC1(C)c2ccccc2-c2cc(N(c3ccc(-c4cccc5c4ccc4ccccc45)cc3)c3ccccc3-c3ccc4oc5ccccc5c4c3)ccc21 ; CC1(C)c2ccccc2-c2cc(-c3ccccc3N(c3ccc(-c4cccc5c4ccc4ccccc45)cc3)c3ccccc3-c3ccc4oc5ccccc5c4c3)ccc21 ; CC1(C)c2ccccc2-c2c(-c3ccccc3N(c3ccc(-c4cccc5c4ccc4ccccc45)cc3)c3ccccc3-c3ccc4oc5ccccc5c4c3)cccc21]

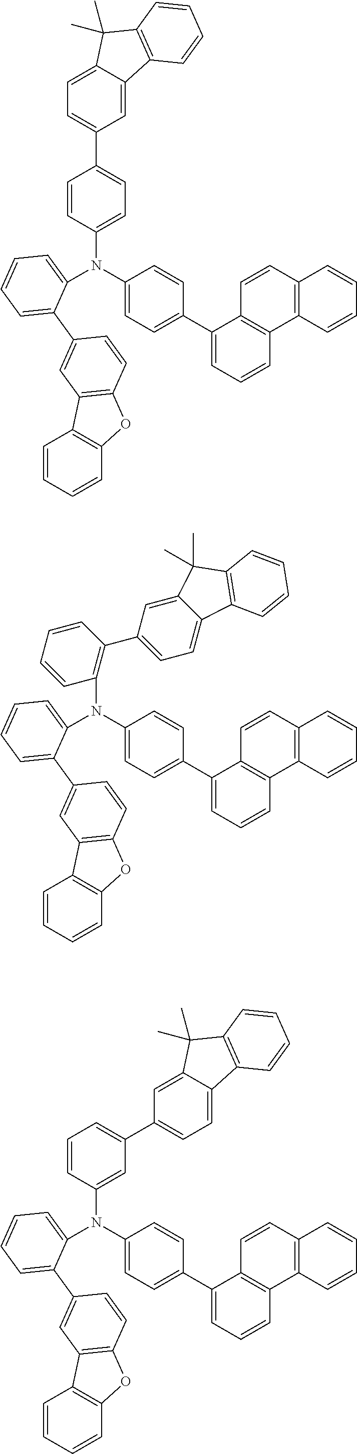 [SMILES: CC1(C)c2ccccc2-c2cc(-c3ccc(N(c4ccc(-c5cccc6c5ccc5ccccc56)cc4)c4ccccc4-c4ccc5oc6ccccc6c5c4)cc3)ccc21 ; CC1(C)c2ccccc2-c2ccc(-c3ccccc3N(c3ccc(-c4cccc5c4ccc4ccccc45)cc3)c3ccccc3-c3ccc4oc5ccccc5c4c3)cc21 ; CC1(C)c2ccccc2-c2ccc(-c3cccc(N(c4ccc(-c5cccc6c5ccc5ccccc56)cc4)c4ccccc4-c4ccc5oc6ccccc6c5c4)c3)cc21]

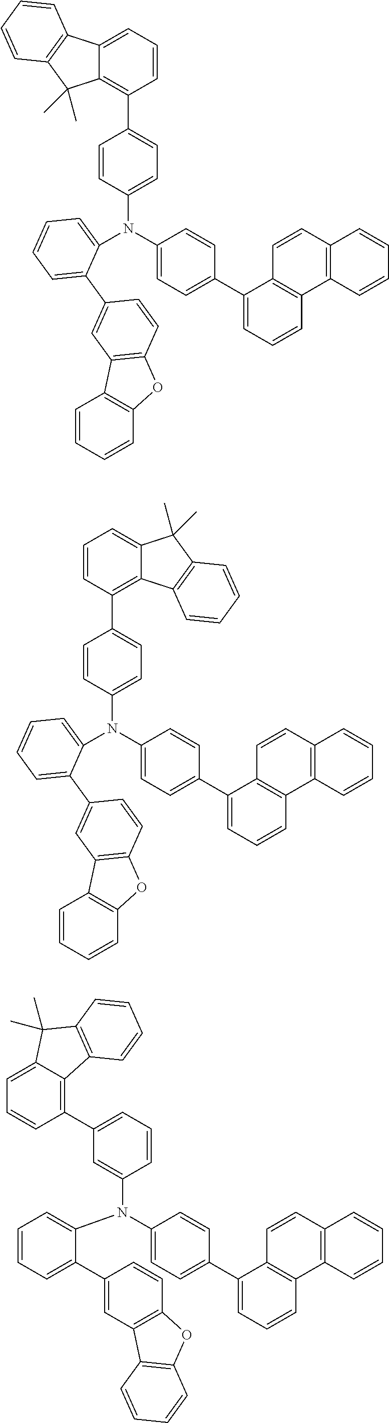

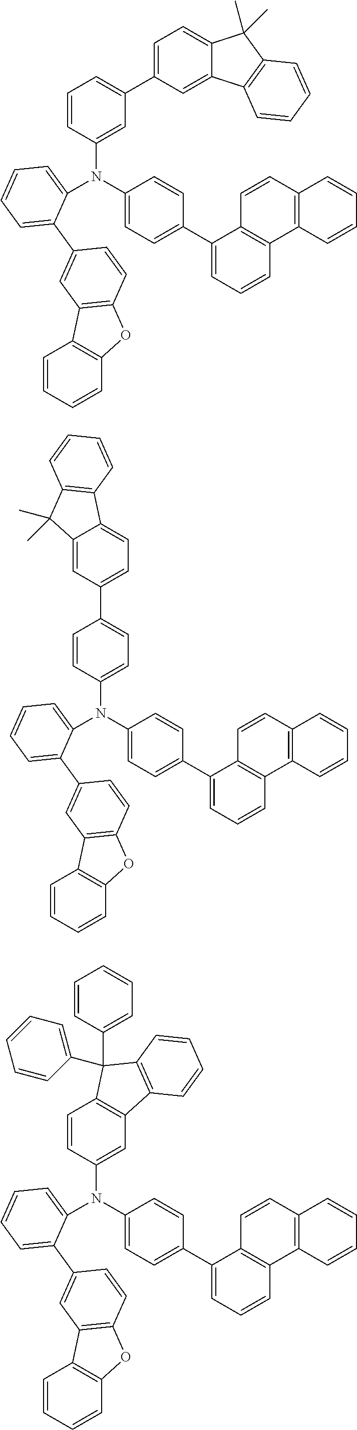

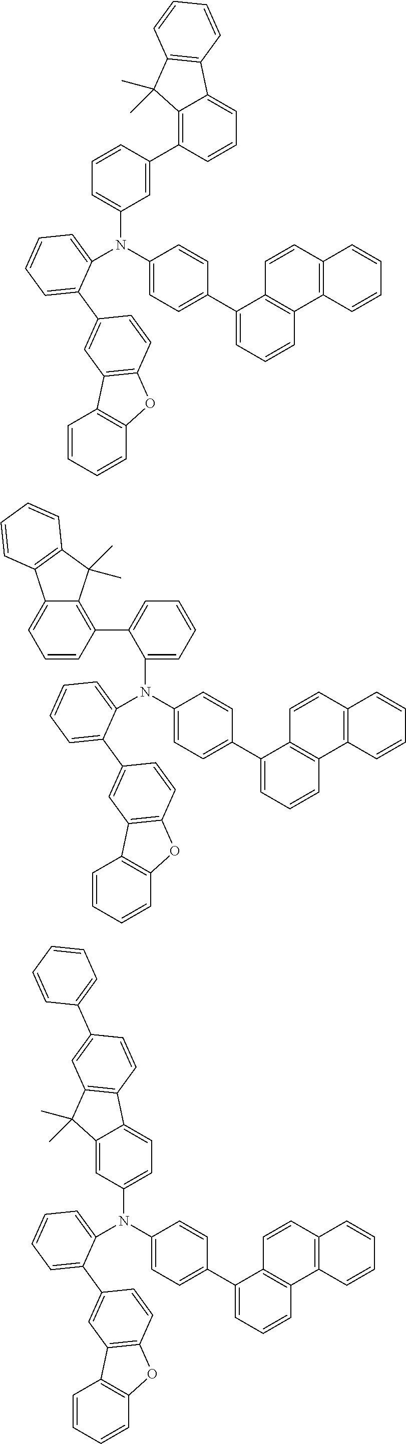

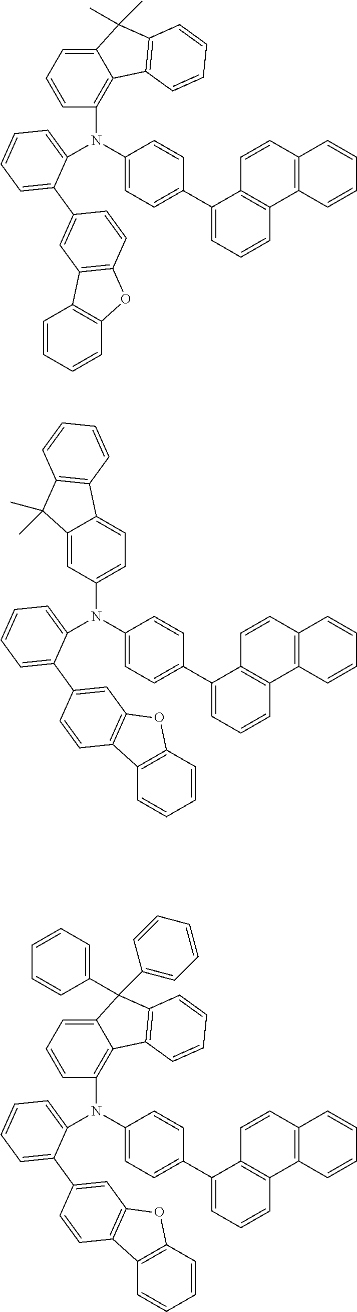 [SMILES: CC1(C)c2ccccc2-c2c(N(c3ccc(-c4cccc5c4ccc4ccccc45)cc3)c3ccccc3-c3ccc4oc5ccccc5c4c3)cccc21 ; CC1(C)c2ccccc2-c2ccc(N(c3ccc(-c4cccc5c4ccc4ccccc45)cc3)c3ccccc3-c3ccc4c(c3)oc3ccccc34)cc21 ; c1ccc(C2(c3ccccc3)c3ccccc3-c3c(N(c4ccc(-c5cccc6c5ccc5ccccc56)cc4)c4ccccc4-c4ccc5c(c4)oc4ccccc45)cccc32)cc1]

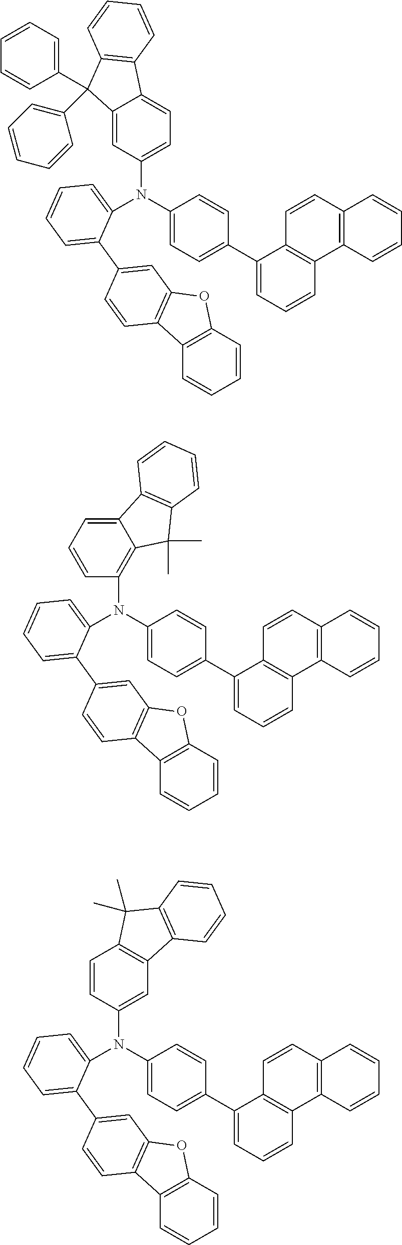 [SMILES: c1ccc(C2(c3ccccc3)c3ccccc3-c3ccc(N(c4ccc(-c5cccc6c5ccc5ccccc56)cc4)c4ccccc4-c4ccc5c(c4)oc4ccccc45)cc32)cc1 ; CC1(C)c2ccccc2-c2cccc(N(c3ccc(-c4cccc5c4ccc4ccccc45)cc3)c3ccccc3-c3ccc4c(c3)oc3ccccc34)c21 ; CC1(C)c2ccccc2-c2cc(N(c3ccc(-c4cccc5c4ccc4ccccc45)cc3)c3ccccc3-c3ccc4c(c3)oc3ccccc34)ccc21]

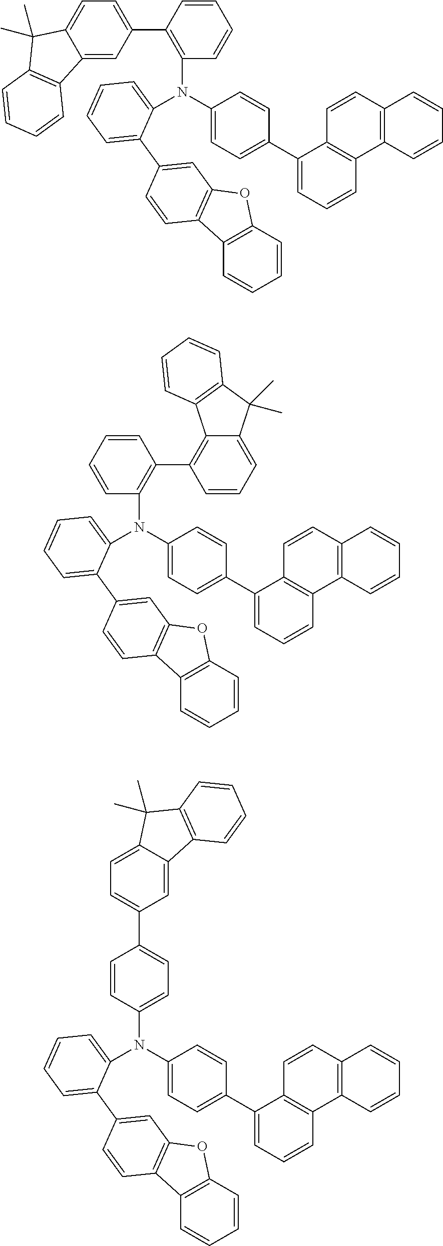 [SMILES: CC1(C)c2ccccc2-c2cc(-c3ccccc3N(c3ccc(-c4cccc5c4ccc4ccccc45)cc3)c3ccccc3-c3ccc4c(c3)oc3ccccc34)ccc21 ; CC1(C)c2ccccc2-c2c(-c3ccccc3N(c3ccc(-c4cccc5c4ccc4ccccc45)cc3)c3ccccc3-c3ccc4c(c3)oc3ccccc34)cccc21 ; CC1(C)c2ccccc2-c2cc(-c3ccc(N(c4ccc(-c5cccc6c5ccc5ccccc56)cc4)c4ccccc4-c4ccc5c(c4)oc4ccccc45)cc3)ccc21]

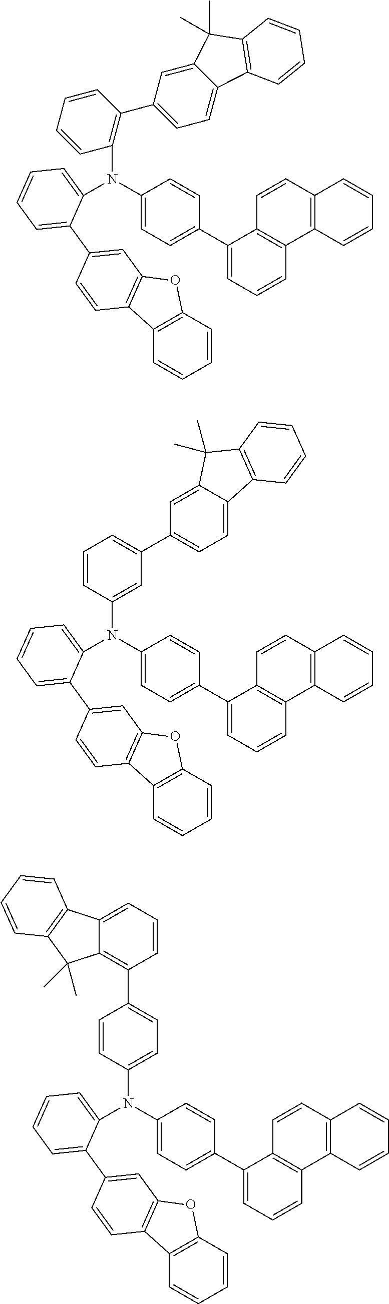

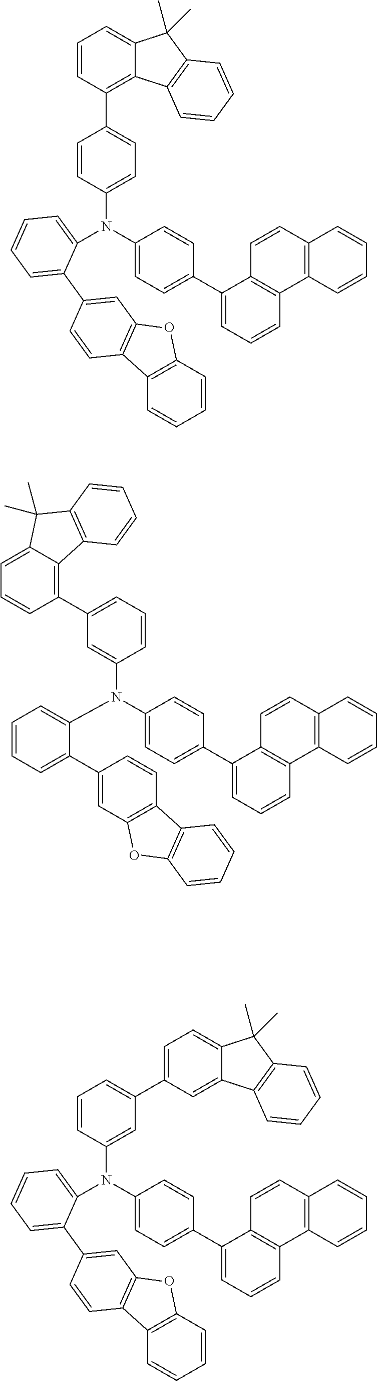 [SMILES: CC1(C)c2ccccc2-c2c(-c3ccc(N(c4ccc(-c5cccc6c5ccc5ccccc56)cc4)c4ccccc4-c4ccc5c(c4)oc4ccccc45)cc3)cccc21 ; CC1(C)c2ccccc2-c2c(-c3cccc(N(c4ccc(-c5cccc6c5ccc5ccccc56)cc4)c4ccccc4-c4ccc5c(c4)oc4ccccc45)c3)cccc21 ; CC1(C)c2ccccc2-c2cc(-c3cccc(N(c4ccc(-c5cccc6c5ccc5ccccc56)cc4)c4ccccc4-c4ccc5c(c4)oc4ccccc45)c3)ccc21]

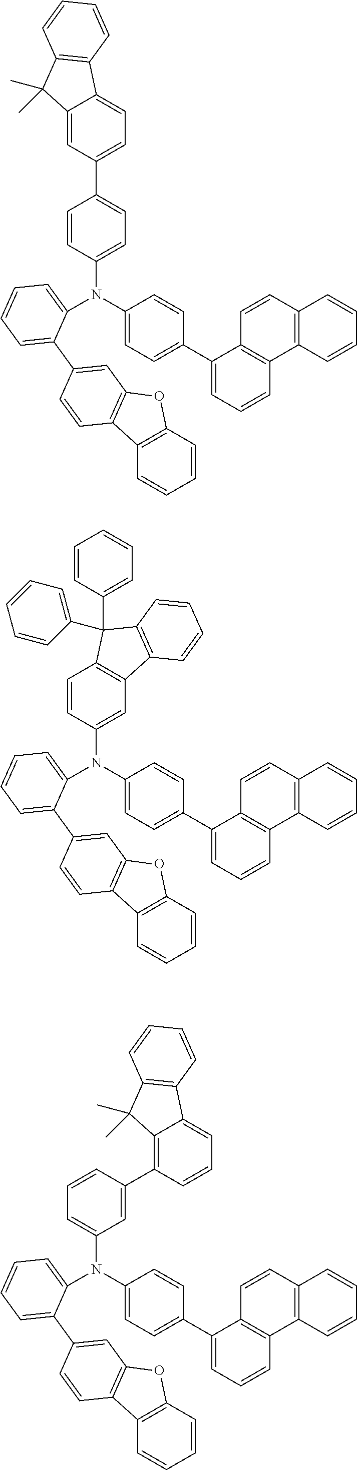 [SMILES: CC1(C)c2ccccc2-c2ccc(-c3ccc(N(c4ccc(-c5cccc6c5ccc5ccccc56)cc4)c4ccccc4-c4ccc5c(c4)oc4ccccc45)cc3)cc21 ; c1ccc(C2(c3ccccc3)c3ccccc3-c3cc(N(c4ccc(-c5cccc6c5ccc5ccccc56)cc4)c4ccccc4-c4ccc5c(c4)oc4ccccc45)ccc32)cc1 ; CC1(C)c2ccccc2-c2cccc(-c3cccc(N(c4ccc(-c5cccc6c5ccc5ccccc56)cc4)c4ccccc4-c4ccc5c(c4)oc4ccccc45)c3)c21]

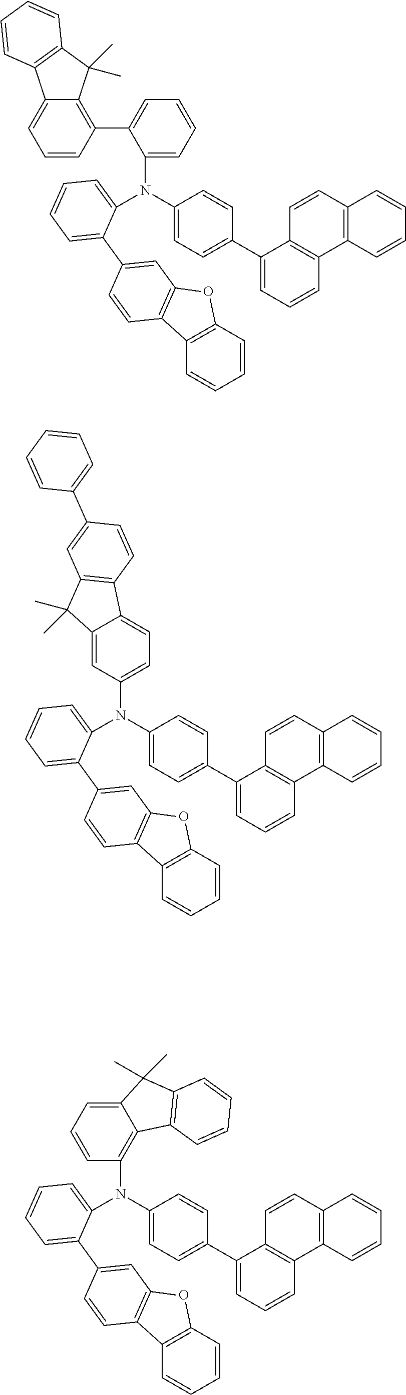 [SMILES: CC1(C)c2ccccc2-c2cccc(-c3ccccc3N(c3ccc(-c4cccc5c4ccc4ccccc45)cc3)c3ccccc3-c3ccc4c(c3)oc3ccccc34)c21 ; CC1(C)c2cc(-c3ccccc3)ccc2-c2ccc(N(c3ccc(-c4cccc5c4ccc4ccccc45)cc3)c3ccccc3-c3ccc4c(c3)oc3ccccc34)cc21 ; CC1(C)c2ccccc2-c2c(N(c3ccc(-c4cccc5c4ccc4ccccc45)cc3)c3ccccc3-c3ccc4c(c3)oc3ccccc34)cccc21]

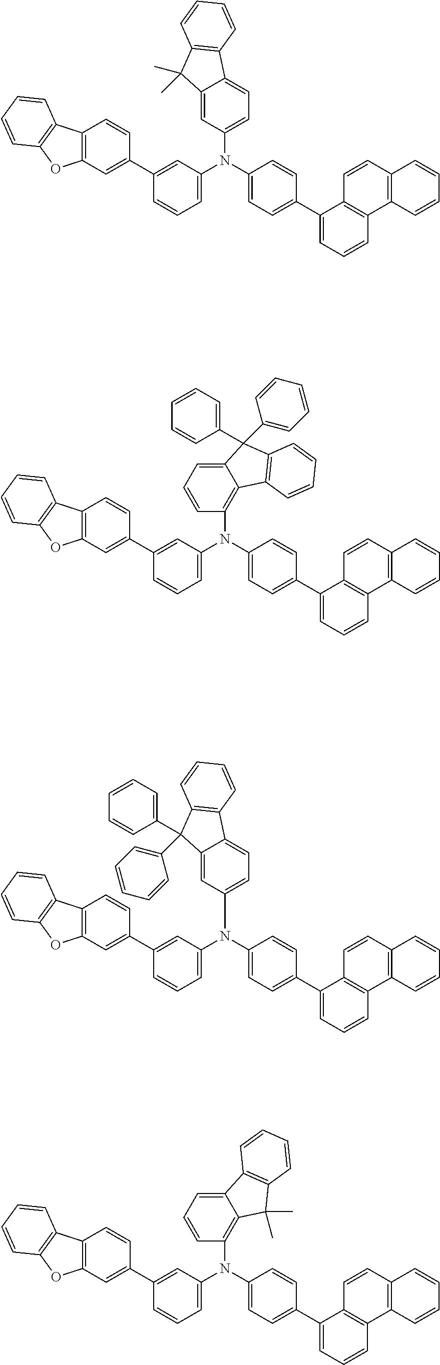 [SMILES: CC1(C)c2ccccc2-c2ccc(N(c3ccc(-c4cccc5c4ccc4ccccc45)cc3)c3cccc(-c4ccc5c(c4)oc4ccccc45)c3)cc21 ; c1ccc(C2(c3ccccc3)c3ccccc3-c3c(N(c4ccc(-c5cccc6c5ccc5ccccc56)cc4)c4cccc(-c5ccc6c(c5)oc5ccccc56)c4)cccc32)cc1 ; c1ccc(C2(c3ccccc3)c3ccccc3-c3ccc(N(c4ccc(-c5cccc6c5ccc5ccccc56)cc4)c4cccc(-c5ccc6c(c5)oc5ccccc56)c4)cc32)cc1 ; CC1(C)c2ccccc2-c2cccc(N(c3ccc(-c4cccc5c4ccc4ccccc45)cc3)c3cccc(-c4ccc5c(c4)oc4ccccc45)c3)c21]

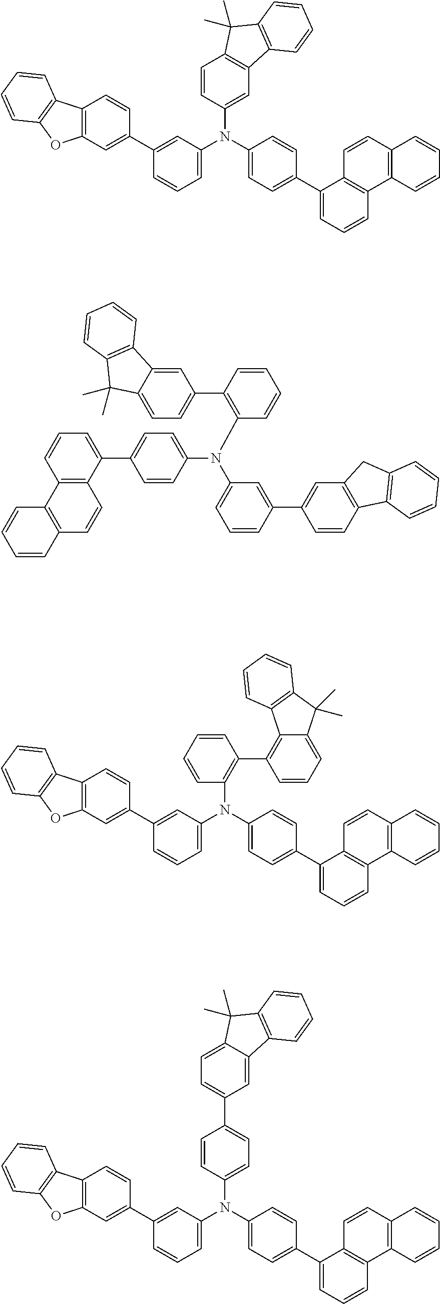 [SMILES: CC1(C)c2ccccc2-c2cc(N(c3ccc(-c4cccc5c4ccc4ccccc45)cc3)c3cccc(-c4ccc5c(c4)oc4ccccc45)c3)ccc21 ; CC1(C)c2ccccc2-c2cc(-c3ccccc3N(c3ccc(-c4cccc5c4ccc4ccccc45)cc3)c3cccc(-c4ccc5c(c4)Cc4ccccc4-5)c3)ccc21 ; CC1(C)c2ccccc2-c2c(-c3ccccc3N(c3ccc(-c4cccc5c4ccc4ccccc45)cc3)c3cccc(-c4ccc5c(c4)oc4ccccc45)c3)cccc21 ; CC1(C)c2ccccc2-c2cc(-c3ccc(N(c4ccc(-c5cccc6c5ccc5ccccc56)cc4)c4cccc(-c5ccc6c(c5)oc5ccccc56)c4)cc3)ccc21]

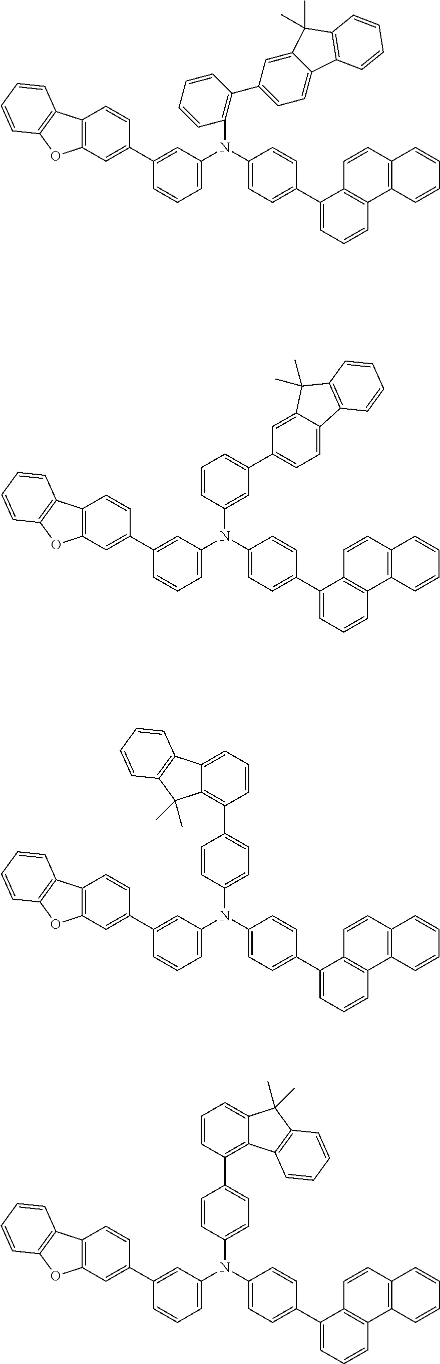 [SMILES: CC1(C)c2ccccc2-c2ccc(-c3ccccc3N(c3ccc(-c4cccc5c4ccc4ccccc45)cc3)c3cccc(-c4ccc5c(c4)oc4ccccc45)c3)cc21 ; CC1(C)c2ccccc2-c2ccc(-c3cccc(N(c4ccc(-c5cccc6c5ccc5ccccc56)cc4)c4cccc(-c5ccc6c(c5)oc5ccccc56)c4)c3)cc21 ; CC1(C)c2ccccc2-c2cccc(-c3ccc(N(c4ccc(-c5cccc6c5ccc5ccccc56)cc4)c4cccc(-c5ccc6c(c5)oc5ccccc56)c4)cc3)c21 ; CC1(C)c2ccccc2-c2c(-c3ccc(N(c4ccc(-c5cccc6c5ccc5ccccc56)cc4)c4cccc(-c5ccc6c(c5)oc5ccccc56)c4)cc3)cccc21]

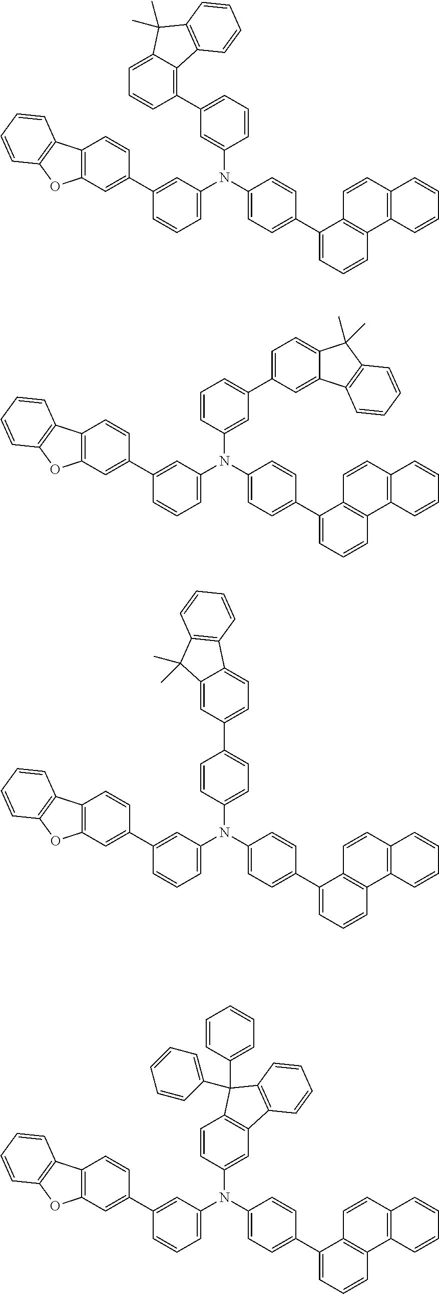 [SMILES: CC1(C)c2ccccc2-c2c(-c3cccc(N(c4ccc(-c5cccc6c5ccc5ccccc56)cc4)c4cccc(-c5ccc6c(c5)oc5ccccc56)c4)c3)cccc21 ; CC1(C)c2ccccc2-c2cc(-c3cccc(N(c4ccc(-c5cccc6c5ccc5ccccc56)cc4)c4cccc(-c5ccc6c(c5)oc5ccccc56)c4)c3)ccc21 ; CC1(C)c2ccccc2-c2ccc(-c3ccc(N(c4ccc(-c5cccc6c5ccc5ccccc56)cc4)c4cccc(-c5ccc6c(c5)oc5ccccc56)c4)cc3)cc21 ; c1ccc(C2(c3ccccc3)c3ccccc3-c3cc(N(c4ccc(-c5cccc6c5ccc5ccccc56)cc4)c4cccc(-c5ccc6c(c5)oc5ccccc56)c4)ccc32)cc1]

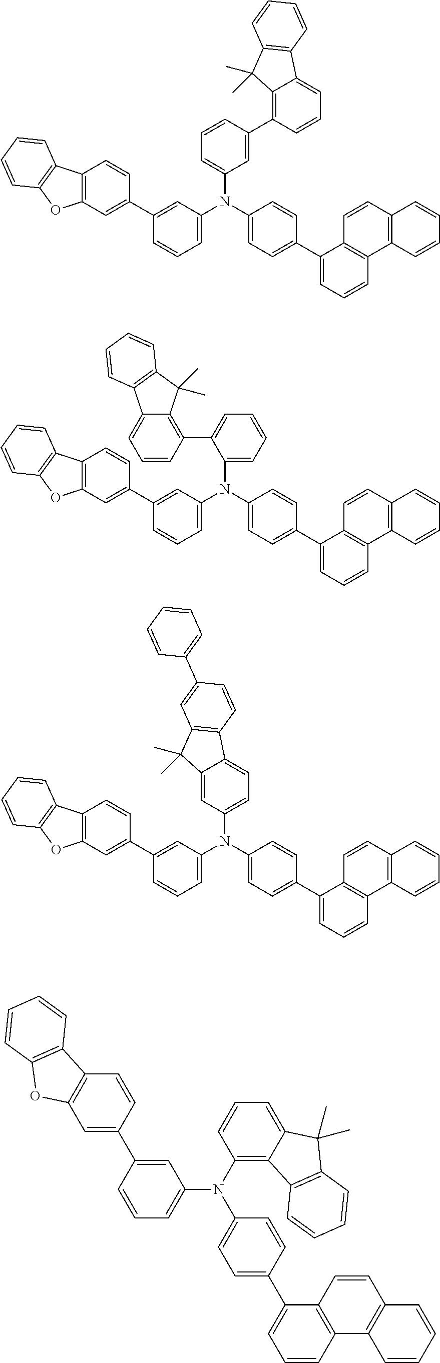 [SMILES: CC1(C)c2ccccc2-c2cccc(-c3cccc(N(c4ccc(-c5cccc6c5ccc5ccccc56)cc4)c4cccc(-c5ccc6c(c5)oc5ccccc56)c4)c3)c21 ; CC1(C)c2ccccc2-c2cccc(-c3ccccc3N(c3ccc(-c4cccc5c4ccc4ccccc45)cc3)c3cccc(-c4ccc5c(c4)oc4ccccc45)c3)c21 ; CC1(C)c2cc(-c3ccccc3)ccc2-c2ccc(N(c3ccc(-c4cccc5c4ccc4ccccc45)cc3)c3cccc(-c4ccc5c(c4)oc4ccccc45)c3)cc21 ; CC1(C)c2ccccc2-c2c(N(c3ccc(-c4cccc5c4ccc4ccccc45)cc3)c3cccc(-c4ccc5c(c4)oc4ccccc45)c3)cccc21]

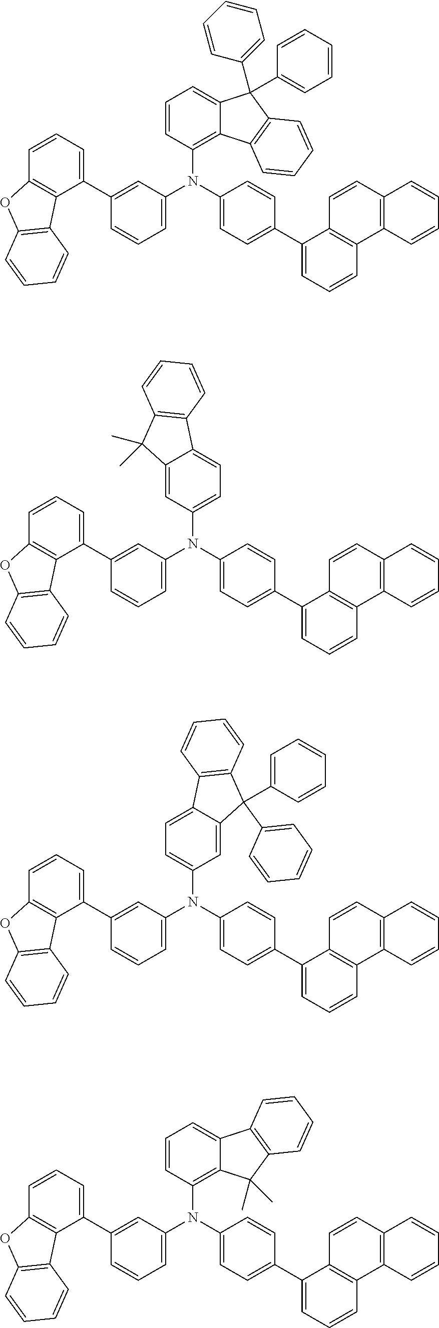 [SMILES: c1ccc(C2(c3ccccc3)c3ccccc3-c3c(N(c4ccc(-c5cccc6c5ccc5ccccc56)cc4)c4cccc(-c5cccc6oc7ccccc7c56)c4)cccc32)cc1 ; CC1(C)c2ccccc2-c2ccc(N(c3ccc(-c4cccc5c4ccc4ccccc45)cc3)c3cccc(-c4cccc5oc6ccccc6c45)c3)cc21 ; c1ccc(C2(c3ccccc3)c3ccccc3-c3ccc(N(c4ccc(-c5cccc6c5ccc5ccccc56)cc4)c4cccc(-c5cccc6oc7ccccc7c56)c4)cc32)cc1 ; CC1(C)c2ccccc2-c2cccc(N(c3ccc(-c4cccc5c4ccc4ccccc45)cc3)c3cccc(-c4cccc5oc6ccccc6c45)c3)c21]

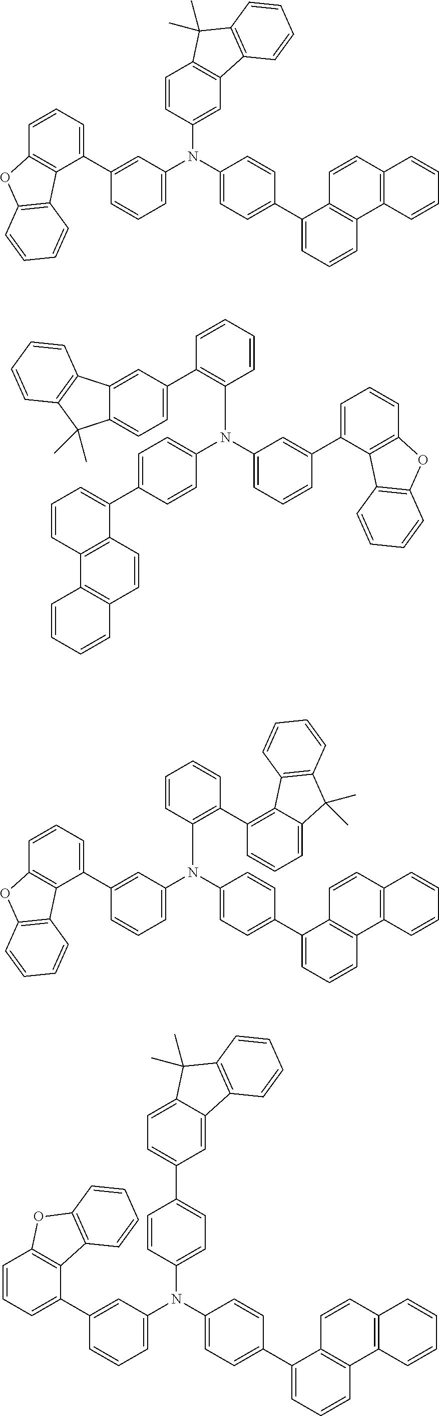 [SMILES: CC1(C)c2ccccc2-c2cc(N(c3ccc(-c4cccc5c4ccc4ccccc45)cc3)c3cccc(-c4cccc5oc6ccccc6c45)c3)ccc21 ; CC1(C)c2ccccc2-c2cc(-c3ccccc3N(c3ccc(-c4cccc5c4ccc4ccccc45)cc3)c3cccc(-c4cccc5oc6ccccc6c45)c3)ccc21 ; CC1(C)c2ccccc2-c2c(-c3ccccc3N(c3ccc(-c4cccc5c4ccc4ccccc45)cc3)c3cccc(-c4cccc5oc6ccccc6c45)c3)cccc21 ; CC1(C)c2ccccc2-c2cc(-c3ccc(N(c4ccc(-c5cccc6c5ccc5ccccc56)cc4)c4cccc(-c5cccc6oc7ccccc7c56)c4)cc3)ccc21]

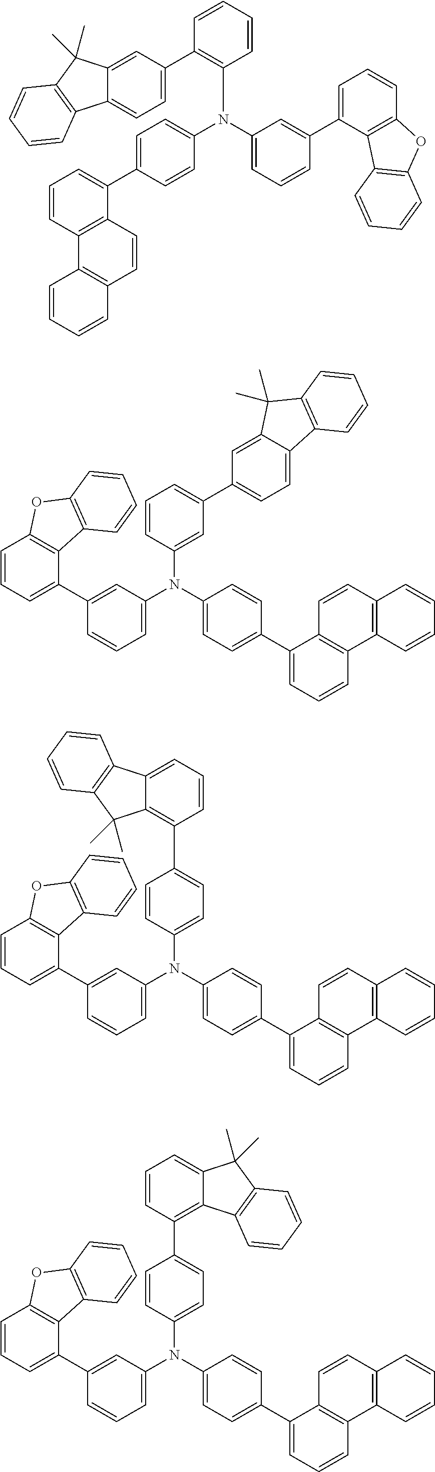 [SMILES: CC1(C)c2ccccc2-c2ccc(-c3ccccc3N(c3ccc(-c4cccc5c4ccc4ccccc45)cc3)c3cccc(-c4cccc5oc6ccccc6c45)c3)cc21 ; CC1(C)c2ccccc2-c2ccc(-c3cccc(N(c4ccc(-c5cccc6c5ccc5ccccc56)cc4)c4cccc(-c5cccc6oc7ccccc7c56)c4)c3)cc21 ; CC1(C)c2ccccc2-c2cccc(-c3ccc(N(c4ccc(-c5cccc6c5ccc5ccccc56)cc4)c4cccc(-c5cccc6oc7ccccc7c56)c4)cc3)c21 ; CC1(C)c2ccccc2-c2c(-c3ccc(N(c4ccc(-c5cccc6c5ccc5ccccc56)cc4)c4cccc(-c5cccc6oc7ccccc7c56)c4)cc3)cccc21]

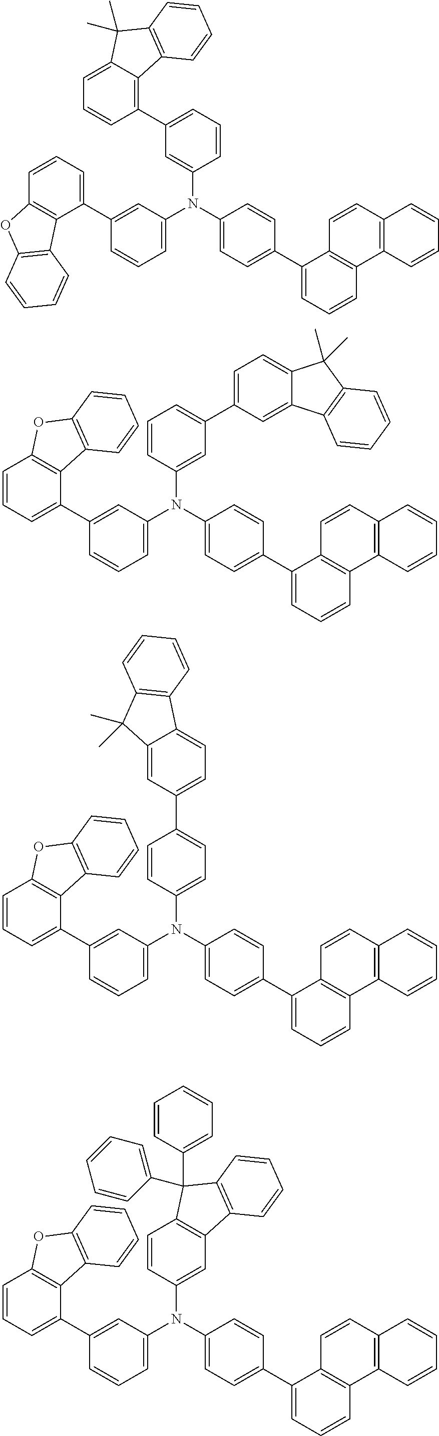

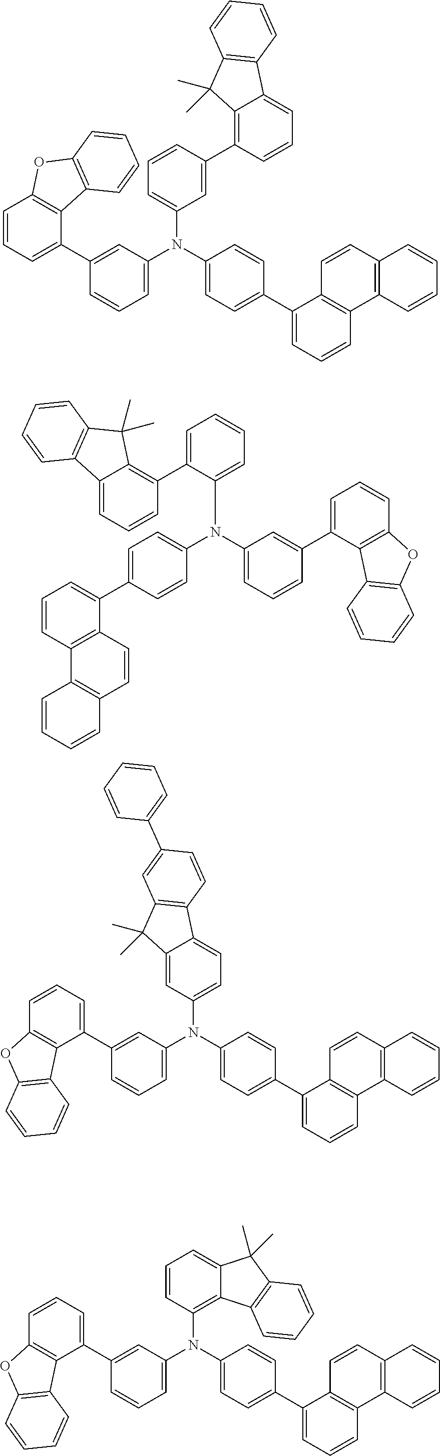

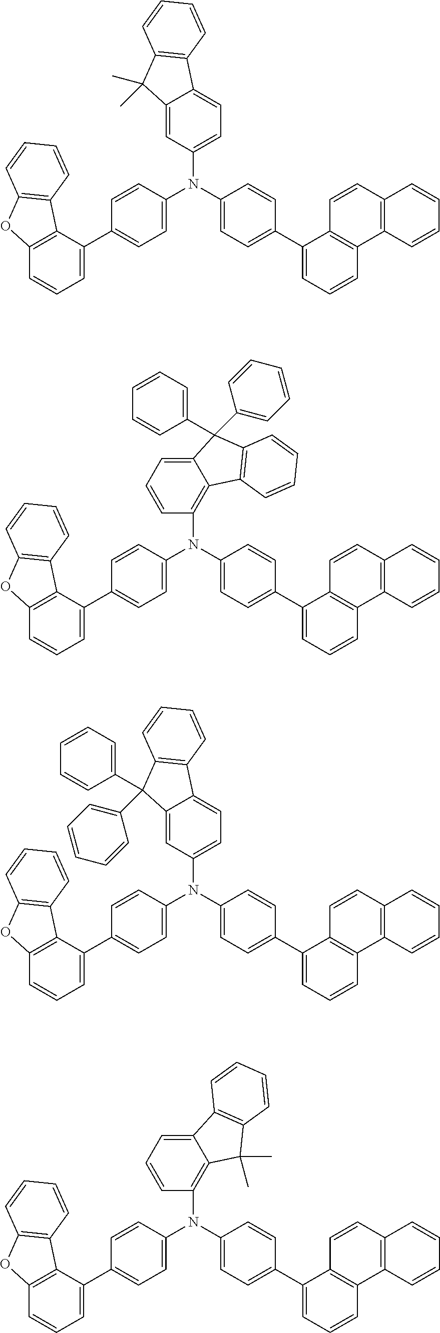 [SMILES: CC1(C)c2ccccc2-c2ccc(N(c3ccc(-c4cccc5c4ccc4ccccc45)cc3)c3ccc(-c4cccc5oc6ccccc6c45)cc3)cc21 ; c1ccc(C2(c3ccccc3)c3ccccc3-c3c(N(c4ccc(-c5cccc6c5ccc5ccccc56)cc4)c4ccc(-c5cccc6oc7ccccc7c56)cc4)cccc32)cc1 ; c1ccc(C2(c3ccccc3)c3ccccc3-c3ccc(N(c4ccc(-c5cccc6c5ccc5ccccc56)cc4)c4ccc(-c5cccc6oc7ccccc7c56)cc4)cc32)cc1 ; CC1(C)c2ccccc2-c2cccc(N(c3ccc(-c4cccc5c4ccc4ccccc45)cc3)c3ccc(-c4cccc5oc6ccccc6c45)cc3)c21]

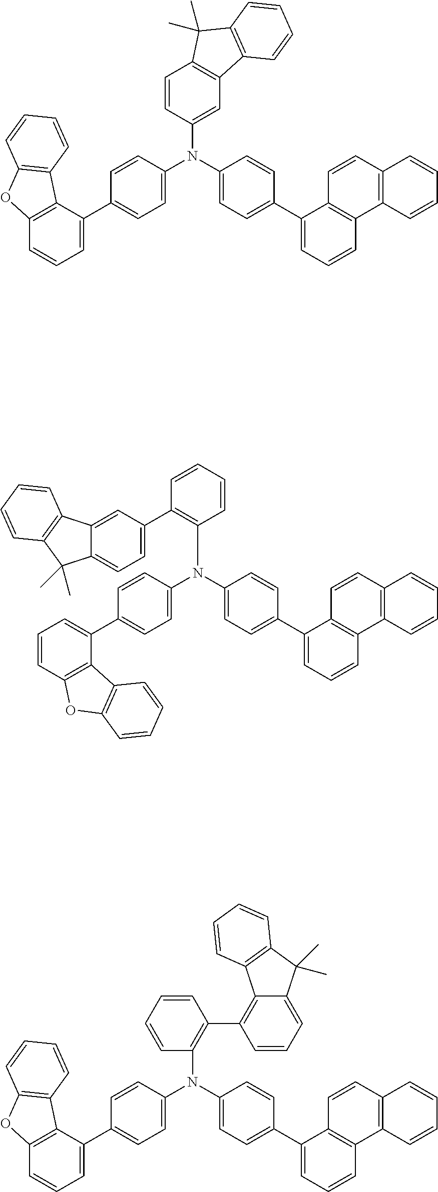 [SMILES: CC1(C)c2ccccc2-c2cc(N(c3ccc(-c4cccc5c4ccc4ccccc45)cc3)c3ccc(-c4cccc5oc6ccccc6c45)cc3)ccc21 ; CC1(C)c2ccccc2-c2cc(-c3ccccc3N(c3ccc(-c4cccc5c4ccc4ccccc45)cc3)c3ccc(-c4cccc5oc6ccccc6c45)cc3)ccc21 ; CC1(C)c2ccccc2-c2c(-c3ccccc3N(c3ccc(-c4cccc5c4ccc4ccccc45)cc3)c3ccc(-c4cccc5oc6ccccc6c45)cc3)cccc21]

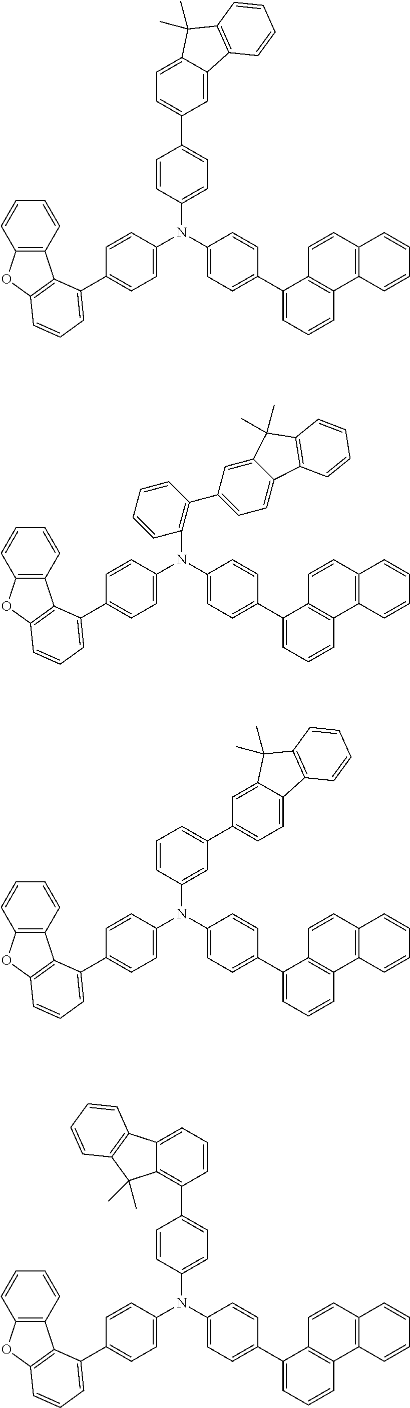 [SMILES: CC1(C)c2ccccc2-c2cc(-c3ccc(N(c4ccc(-c5cccc6c5ccc5ccccc56)cc4)c4ccc(-c5cccc6oc7ccccc7c56)cc4)cc3)ccc21 ; CC1(C)c2ccccc2-c2ccc(-c3ccccc3N(c3ccc(-c4cccc5c4ccc4ccccc45)cc3)c3ccc(-c4cccc5oc6ccccc6c45)cc3)cc21 ; CC1(C)c2ccccc2-c2ccc(-c3cccc(N(c4ccc(-c5cccc6c5ccc5ccccc56)cc4)c4ccc(-c5cccc6oc7ccccc7c56)cc4)c3)cc21 ; CC1(C)c2ccccc2-c2cccc(-c3ccc(N(c4ccc(-c5cccc6c5ccc5ccccc56)cc4)c4ccc(-c5cccc6oc7ccccc7c56)cc4)cc3)c21]

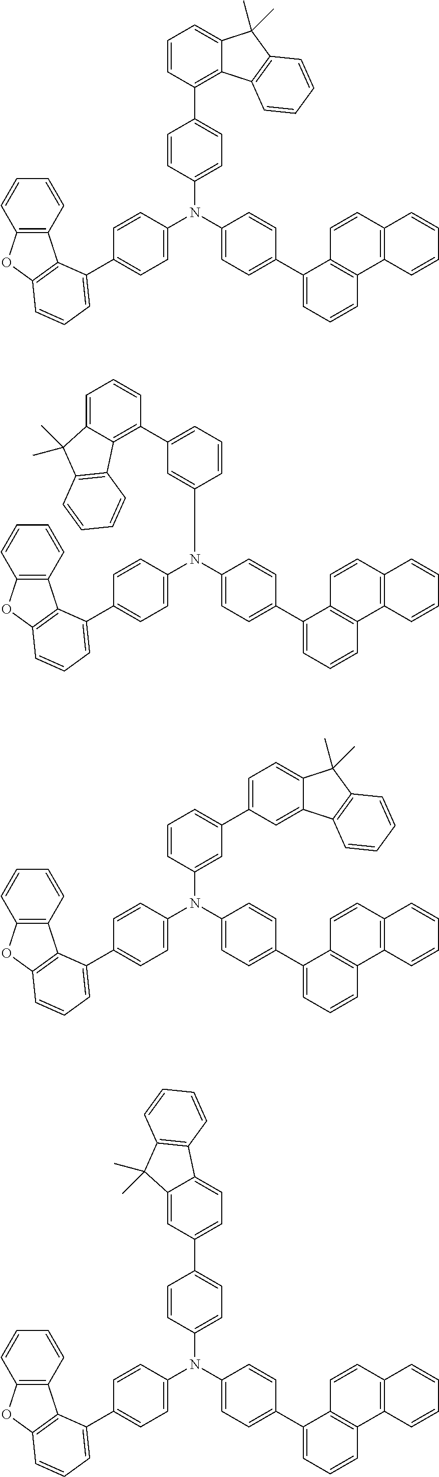 [SMILES: CC1(C)c2ccccc2-c2c(-c3ccc(N(c4ccc(-c5cccc6c5ccc5ccccc56)cc4)c4ccc(-c5cccc6oc7ccccc7c56)cc4)cc3)cccc21 ; CC1(C)c2ccccc2-c2c(-c3cccc(N(c4ccc(-c5cccc6c5ccc5ccccc56)cc4)c4ccc(-c5cccc6oc7ccccc7c56)cc4)c3)cccc21 ; CC1(C)c2ccccc2-c2cc(-c3cccc(N(c4ccc(-c5cccc6c5ccc5ccccc56)cc4)c4ccc(-c5cccc6oc7ccccc7c56)cc4)c3)ccc21 ; CC1(C)c2ccccc2-c2ccc(-c3ccc(N(c4ccc(-c5cccc6c5ccc5ccccc56)cc4)c4ccc(-c5cccc6oc7ccccc7c56)cc4)cc3)cc21]

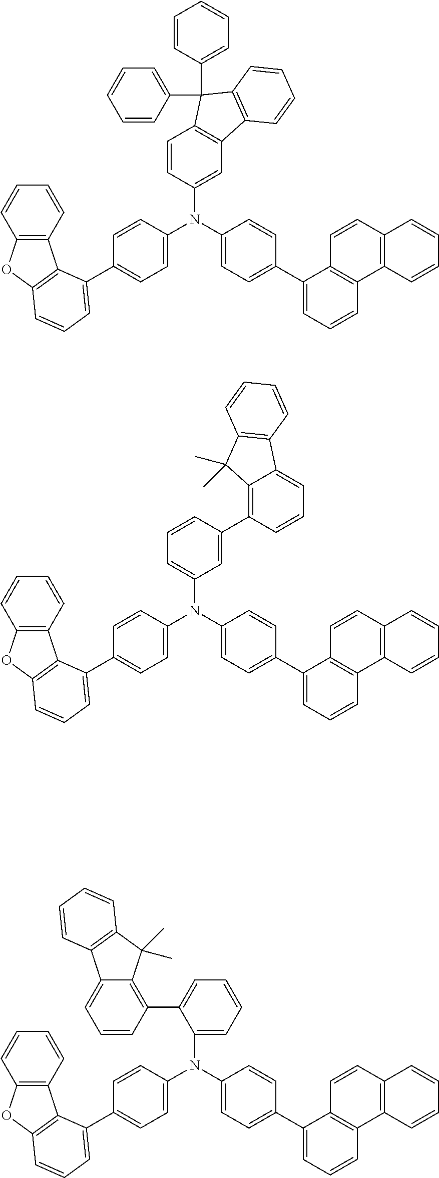 [SMILES: c1ccc(C2(c3ccccc3)c3ccccc3-c3cc(N(c4ccc(-c5cccc6c5ccc5ccccc56)cc4)c4ccc(-c5cccc6oc7ccccc7c56)cc4)ccc32)cc1 ; CC1(C)c2ccccc2-c2cccc(-c3cccc(N(c4ccc(-c5cccc6c5ccc5ccccc56)cc4)c4ccc(-c5cccc6oc7ccccc7c56)cc4)c3)c21 ; CC1(C)c2ccccc2-c2cccc(-c3ccccc3N(c3ccc(-c4cccc5c4ccc4ccccc45)cc3)c3ccc(-c4cccc5oc6ccccc6c45)cc3)c21]

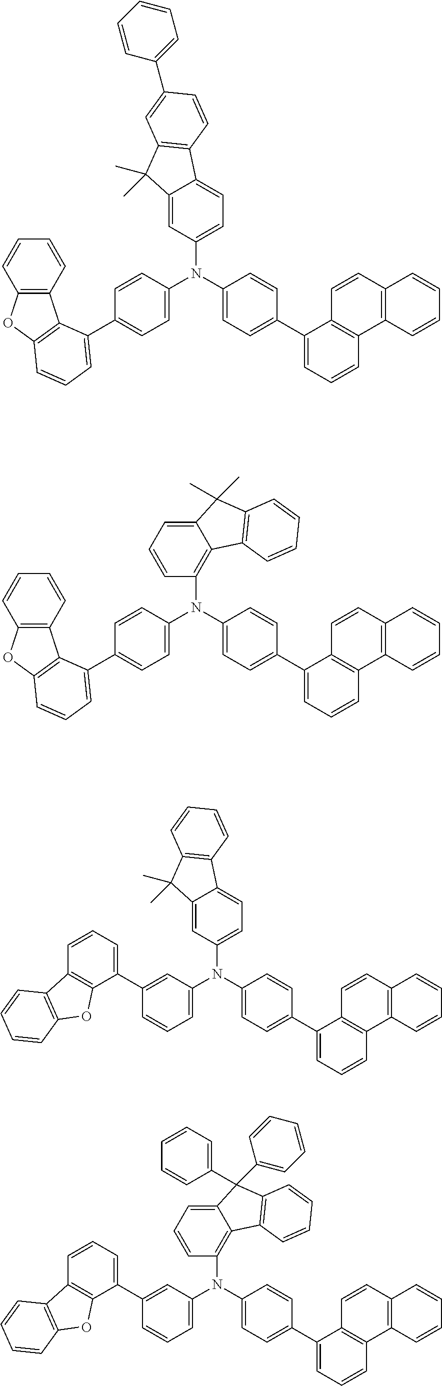 [SMILES: CC1(C)c2cc(-c3ccccc3)ccc2-c2ccc(N(c3ccc(-c4cccc5c4ccc4ccccc45)cc3)c3ccc(-c4cccc5oc6ccccc6c45)cc3)cc21 ; CC1(C)c2ccccc2-c2c(N(c3ccc(-c4cccc5c4ccc4ccccc45)cc3)c3ccc(-c4cccc5oc6ccccc6c45)cc3)cccc21 ; CC1(C)c2ccccc2-c2ccc(N(c3ccc(-c4cccc5c4ccc4ccccc45)cc3)c3cccc(-c4cccc5c4oc4ccccc45)c3)cc21 ; c1ccc(C2(c3ccccc3)c3ccccc3-c3c(N(c4ccc(-c5cccc6c5ccc5ccccc56)cc4)c4cccc(-c5cccc6c5oc5ccccc56)c4)cccc32)cc1]

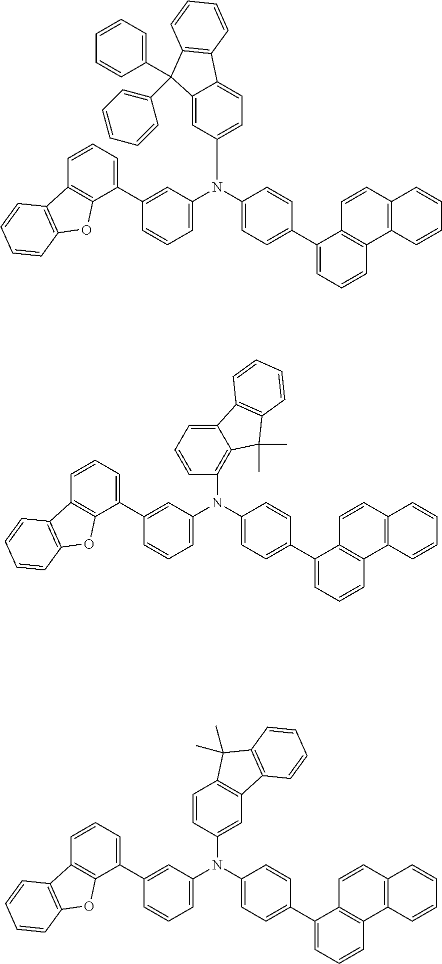 [SMILES: c1ccc(C2(c3ccccc3)c3ccccc3-c3ccc(N(c4ccc(-c5cccc6c5ccc5ccccc56)cc4)c4cccc(-c5cccc6c5oc5ccccc56)c4)cc32)cc1 ; CC1(C)c2ccccc2-c2cccc(N(c3ccc(-c4cccc5c4ccc4ccccc45)cc3)c3cccc(-c4cccc5c4oc4ccccc45)c3)c21 ; CC1(C)c2ccccc2-c2cc(N(c3ccc(-c4cccc5c4ccc4ccccc45)cc3)c3cccc(-c4cccc5c4oc4ccccc45)c3)ccc21]

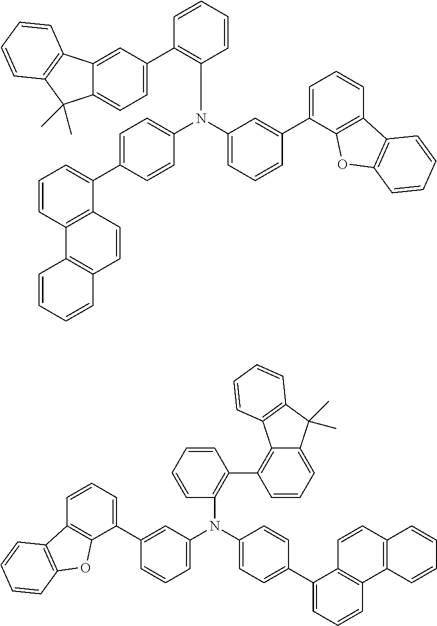 [SMILES: CC1(C)c2ccccc2-c2cc(-c3ccccc3N(c3ccc(-c4cccc5c4ccc4ccccc45)cc3)c3cccc(-c4cccc5c4oc4ccccc45)c3)ccc21 ; CC1(C)c2ccccc2-c2c(-c3ccccc3N(c3ccc(-c4cccc5c4ccc4ccccc45)cc3)c3cccc(-c4cccc5c4oc4ccccc45)c3)cccc21]

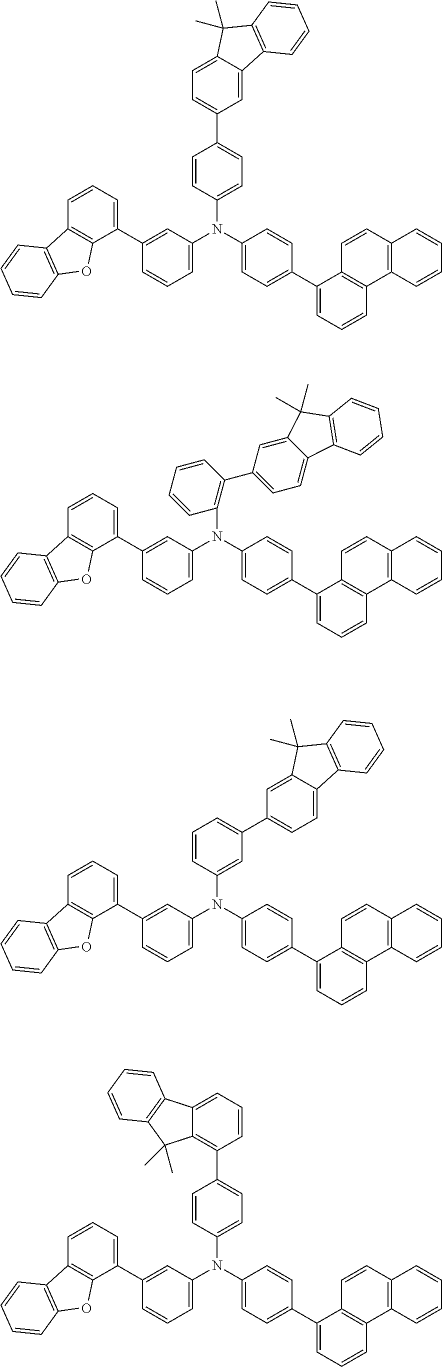 [SMILES: CC1(C)c2ccccc2-c2cc(-c3ccc(N(c4ccc(-c5cccc6c5ccc5ccccc56)cc4)c4cccc(-c5cccc6c5oc5ccccc56)c4)cc3)ccc21 ; CC1(C)c2ccccc2-c2ccc(-c3ccccc3N(c3ccc(-c4cccc5c4ccc4ccccc45)cc3)c3cccc(-c4cccc5c4oc4ccccc45)c3)cc21 ; CC1(C)c2ccccc2-c2ccc(-c3cccc(N(c4ccc(-c5cccc6c5ccc5ccccc56)cc4)c4cccc(-c5cccc6c5oc5ccccc56)c4)c3)cc21 ; CC1(C)c2ccccc2-c2cccc(-c3ccc(N(c4ccc(-c5cccc6c5ccc5ccccc56)cc4)c4cccc(-c5cccc6c5oc5ccccc56)c4)cc3)c21]

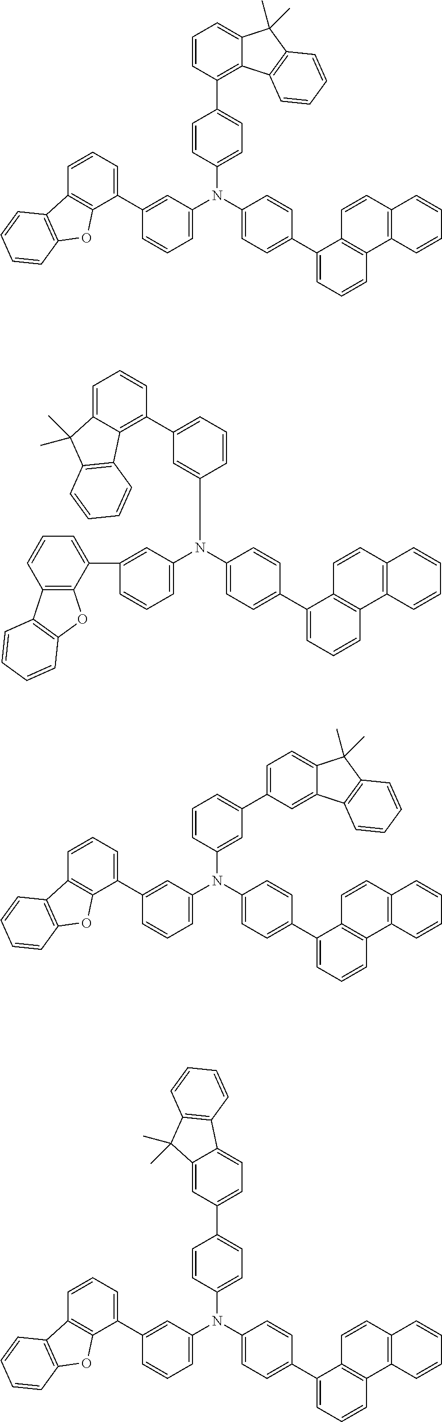 [SMILES: CC1(C)c2ccccc2-c2c(-c3ccc(N(c4ccc(-c5cccc6c5ccc5ccccc56)cc4)c4cccc(-c5cccc6c5oc5ccccc56)c4)cc3)cccc21 ; CC1(C)c2ccccc2-c2c(-c3cccc(N(c4ccc(-c5cccc6c5ccc5ccccc56)cc4)c4cccc(-c5cccc6c5oc5ccccc56)c4)c3)cccc21 ; CC1(C)c2ccccc2-c2cc(-c3cccc(N(c4ccc(-c5cccc6c5ccc5ccccc56)cc4)c4cccc(-c5cccc6c5oc5ccccc56)c4)c3)ccc21 ; CC1(C)c2ccccc2-c2ccc(-c3ccc(N(c4ccc(-c5cccc6c5ccc5ccccc56)cc4)c4cccc(-c5cccc6c5oc5ccccc56)c4)cc3)cc21]

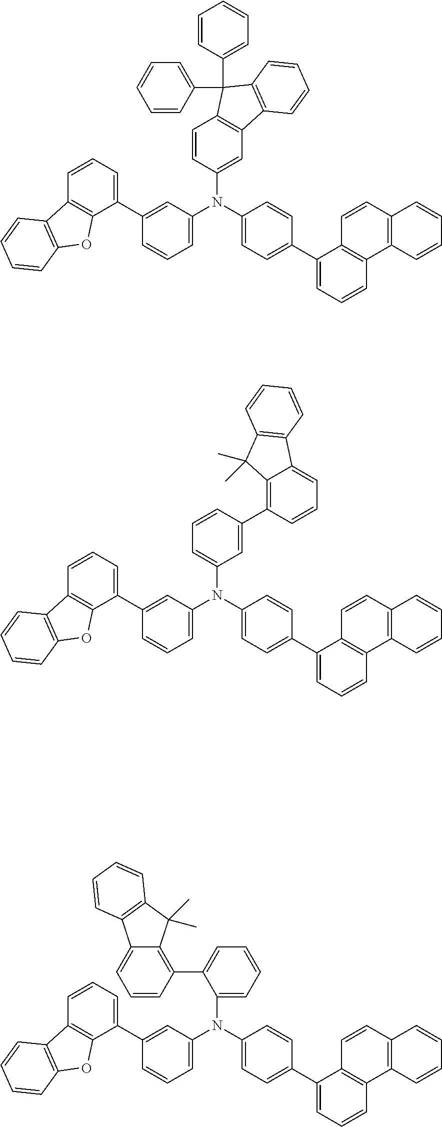 [SMILES: c1ccc(C2(c3ccccc3)c3ccccc3-c3cc(N(c4ccc(-c5cccc6c5ccc5ccccc56)cc4)c4cccc(-c5cccc6c5oc5ccccc56)c4)ccc32)cc1 ; CC1(C)c2ccccc2-c2cccc(-c3cccc(N(c4ccc(-c5cccc6c5ccc5ccccc56)cc4)c4cccc(-c5cccc6c5oc5ccccc56)c4)c3)c21 ; CC1(C)c2ccccc2-c2cccc(-c3ccccc3N(c3ccc(-c4cccc5c4ccc4ccccc45)cc3)c3cccc(-c4cccc5c4oc4ccccc45)c3)c21]

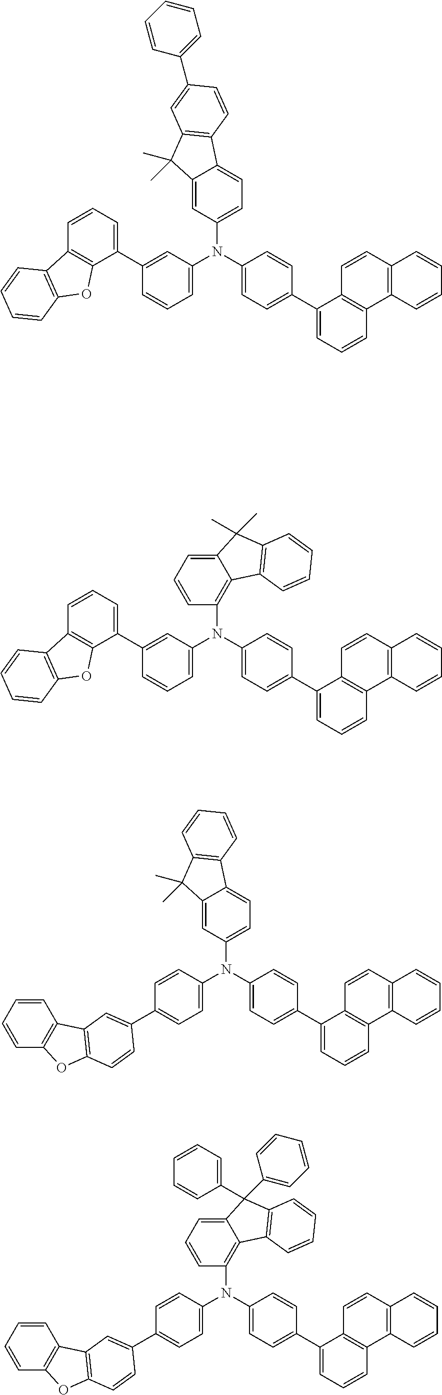 [SMILES: CC1(C)c2cc(-c3ccccc3)ccc2-c2ccc(N(c3ccc(-c4cccc5c4ccc4ccccc45)cc3)c3cccc(-c4cccc5c4oc4ccccc45)c3)cc21 ; CC1(C)c2ccccc2-c2c(N(c3ccc(-c4cccc5c4ccc4ccccc45)cc3)c3cccc(-c4cccc5c4oc4ccccc45)c3)cccc21 ; CC1(C)c2ccccc2-c2ccc(N(c3ccc(-c4ccc5oc6ccccc6c5c4)cc3)c3ccc(-c4cccc5c4ccc4ccccc45)cc3)cc21 ; c1ccc(C2(c3ccccc3)c3ccccc3-c3c(N(c4ccc(-c5ccc6oc7ccccc7c6c5)cc4)c4ccc(-c5cccc6c5ccc5ccccc56)cc4)cccc32)cc1]

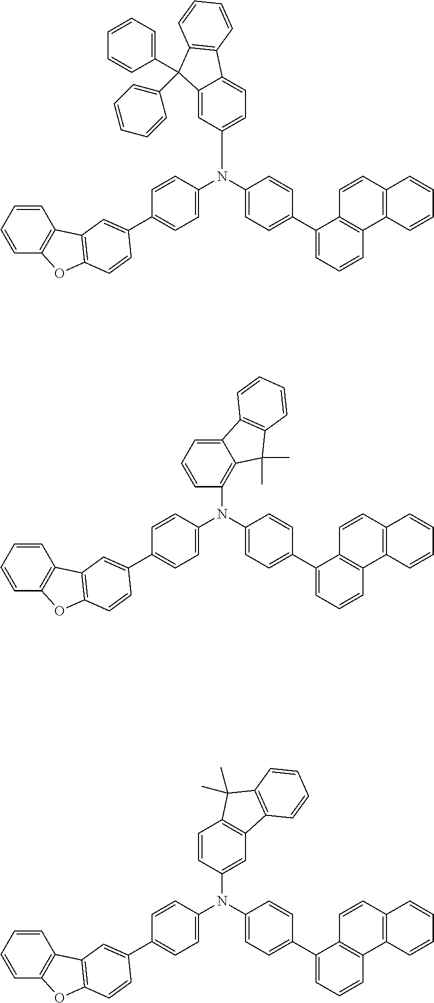 [SMILES: c1ccc(C2(c3ccccc3)c3ccccc3-c3ccc(N(c4ccc(-c5ccc6oc7ccccc7c6c5)cc4)c4ccc(-c5cccc6c5ccc5ccccc56)cc4)cc32)cc1 ; CC1(C)c2ccccc2-c2cccc(N(c3ccc(-c4ccc5oc6ccccc6c5c4)cc3)c3ccc(-c4cccc5c4ccc4ccccc45)cc3)c21 ; CC1(C)c2ccccc2-c2cc(N(c3ccc(-c4ccc5oc6ccccc6c5c4)cc3)c3ccc(-c4cccc5c4ccc4ccccc45)cc3)ccc21]

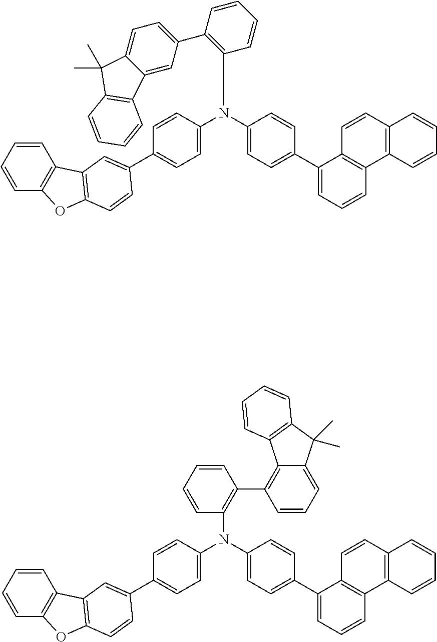 [SMILES: CC1(C)c2ccccc2-c2cc(-c3ccccc3N(c3ccc(-c4ccc5oc6ccccc6c5c4)cc3)c3ccc(-c4cccc5c4ccc4ccccc45)cc3)ccc21 ; CC1(C)c2ccccc2-c2c(-c3ccccc3N(c3ccc(-c4ccc5oc6ccccc6c5c4)cc3)c3ccc(-c4cccc5c4ccc4ccccc45)cc3)cccc21]

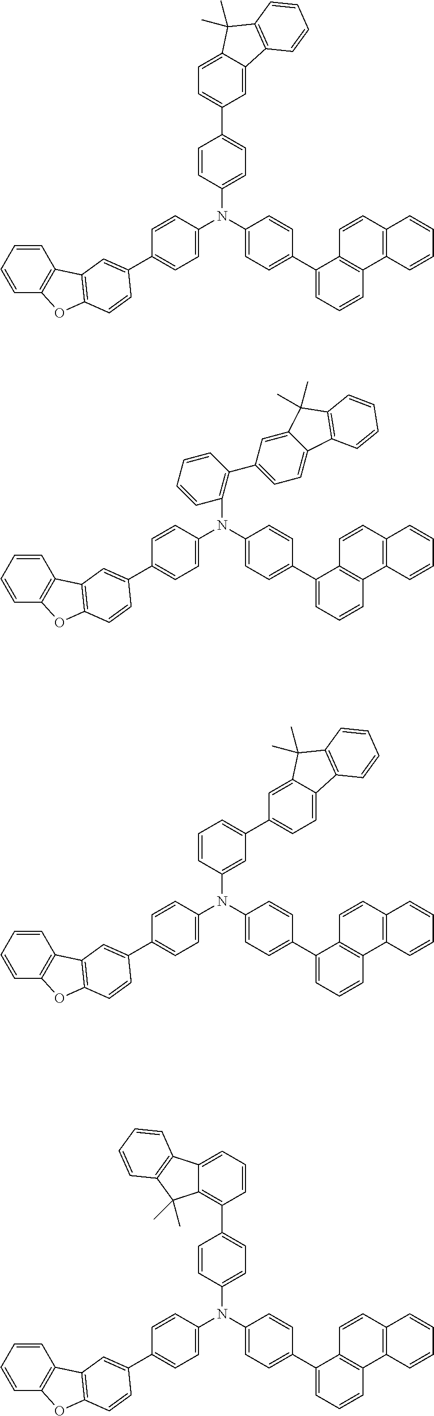 [SMILES: CC1(C)c2ccccc2-c2cc(-c3ccc(N(c4ccc(-c5ccc6oc7ccccc7c6c5)cc4)c4ccc(-c5cccc6c5ccc5ccccc56)cc4)cc3)ccc21 ; CC1(C)c2ccccc2-c2ccc(-c3ccccc3N(c3ccc(-c4ccc5oc6ccccc6c5c4)cc3)c3ccc(-c4cccc5c4ccc4ccccc45)cc3)cc21 ; CC1(C)c2ccccc2-c2ccc(-c3cccc(N(c4ccc(-c5ccc6oc7ccccc7c6c5)cc4)c4ccc(-c5cccc6c5ccc5ccccc56)cc4)c3)cc21 ; CC1(C)c2ccccc2-c2cccc(-c3ccc(N(c4ccc(-c5ccc6oc7ccccc7c6c5)cc4)c4ccc(-c5cccc6c5ccc5ccccc56)cc4)cc3)c21]

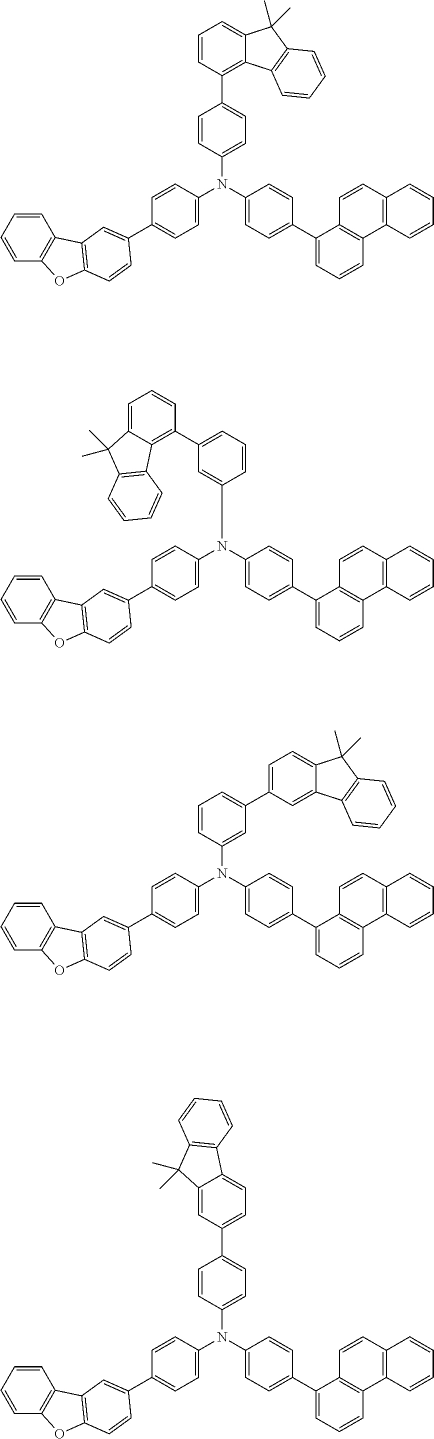 [SMILES: CC1(C)c2ccccc2-c2c(-c3ccc(N(c4ccc(-c5ccc6oc7ccccc7c6c5)cc4)c4ccc(-c5cccc6c5ccc5ccccc56)cc4)cc3)cccc21 ; CC1(C)c2ccccc2-c2c(-c3cccc(N(c4ccc(-c5ccc6oc7ccccc7c6c5)cc4)c4ccc(-c5cccc6c5ccc5ccccc56)cc4)c3)cccc21 ; CC1(C)c2ccccc2-c2cc(-c3cccc(N(c4ccc(-c5ccc6oc7ccccc7c6c5)cc4)c4ccc(-c5cccc6c5ccc5ccccc56)cc4)c3)ccc21 ; CC1(C)c2ccccc2-c2ccc(-c3ccc(N(c4ccc(-c5ccc6oc7ccccc7c6c5)cc4)c4ccc(-c5cccc6c5ccc5ccccc56)cc4)cc3)cc21]

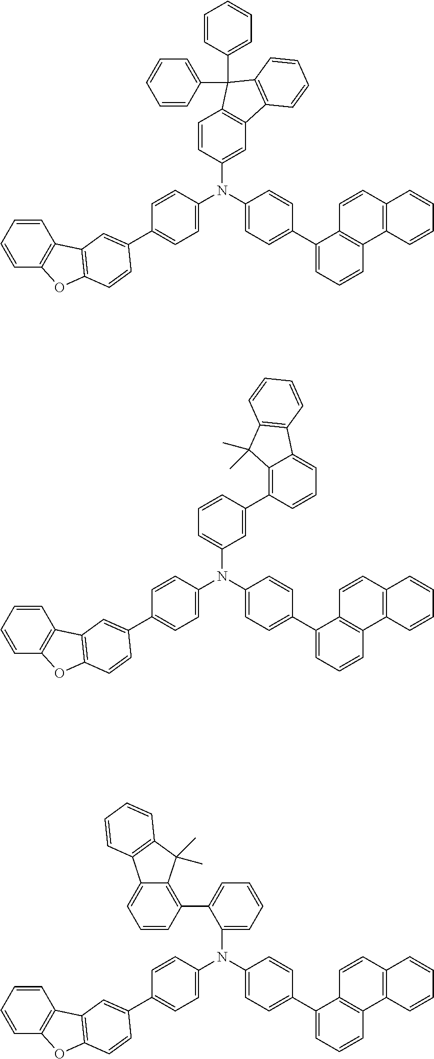 [SMILES: c1ccc(C2(c3ccccc3)c3ccccc3-c3cc(N(c4ccc(-c5ccc6oc7ccccc7c6c5)cc4)c4ccc(-c5cccc6c5ccc5ccccc56)cc4)ccc32)cc1 ; CC1(C)c2ccccc2-c2cccc(-c3cccc(N(c4ccc(-c5ccc6oc7ccccc7c6c5)cc4)c4ccc(-c5cccc6c5ccc5ccccc56)cc4)c3)c21 ; CC1(C)c2ccccc2-c2cccc(-c3ccccc3N(c3ccc(-c4ccc5oc6ccccc6c5c4)cc3)c3ccc(-c4cccc5c4ccc4ccccc45)cc3)c21]

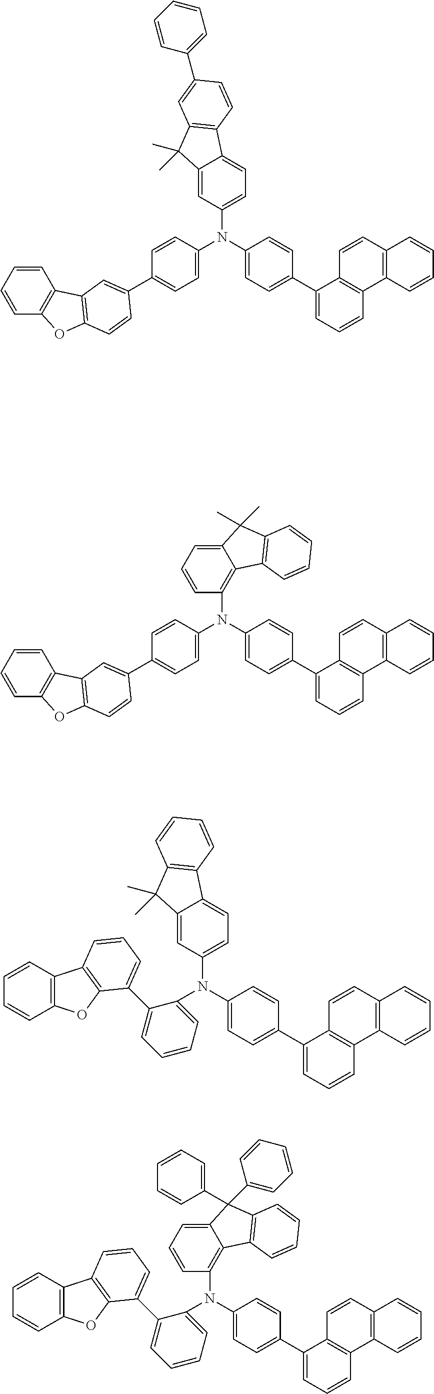 [SMILES: CC1(C)c2cc(-c3ccccc3)ccc2-c2ccc(N(c3ccc(-c4ccc5oc6ccccc6c5c4)cc3)c3ccc(-c4cccc5c4ccc4ccccc45)cc3)cc21 ; CC1(C)c2ccccc2-c2c(N(c3ccc(-c4ccc5oc6ccccc6c5c4)cc3)c3ccc(-c4cccc5c4ccc4ccccc45)cc3)cccc21 ; CC1(C)c2ccccc2-c2ccc(N(c3ccc(-c4cccc5c4ccc4ccccc45)cc3)c3ccccc3-c3cccc4c3oc3ccccc34)cc21 ; c1ccc(C2(c3ccccc3)c3ccccc3-c3c(N(c4ccc(-c5cccc6c5ccc5ccccc56)cc4)c4ccccc4-c4cccc5c4oc4ccccc45)cccc32)cc1]

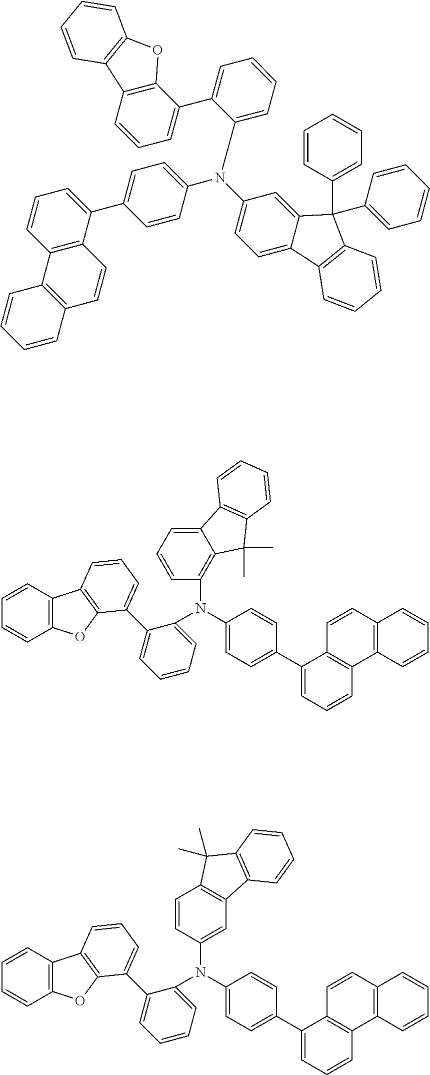 [SMILES: c1ccc(C2(c3ccccc3)c3ccccc3-c3ccc(N(c4ccc(-c5cccc6c5ccc5ccccc56)cc4)c4ccccc4-c4cccc5c4oc4ccccc45)cc32)cc1 ; CC1(C)c2ccccc2-c2cccc(N(c3ccc(-c4cccc5c4ccc4ccccc45)cc3)c3ccccc3-c3cccc4c3oc3ccccc34)c21 ; CC1(C)c2ccccc2-c2cc(N(c3ccc(-c4cccc5c4ccc4ccccc45)cc3)c3ccccc3-c3cccc4c3oc3ccccc34)ccc21]

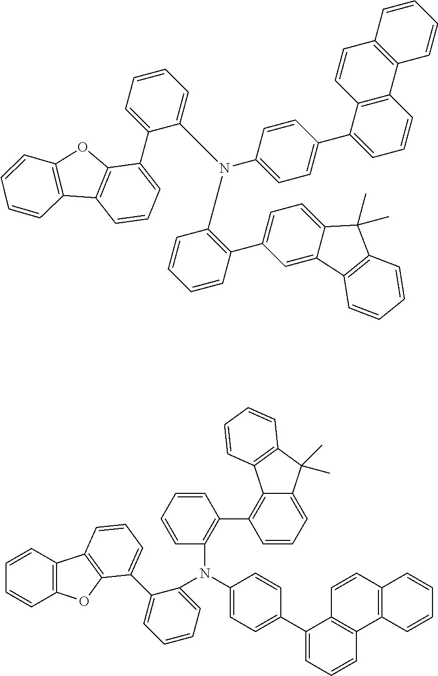 [SMILES: CC1(C)c2ccccc2-c2cc(-c3ccccc3N(c3ccc(-c4cccc5c4ccc4ccccc45)cc3)c3ccccc3-c3cccc4c3oc3ccccc34)ccc21 ; CC1(C)c2ccccc2-c2c(-c3ccccc3N(c3ccc(-c4cccc5c4ccc4ccccc45)cc3)c3ccccc3-c3cccc4c3oc3ccccc34)cccc21]

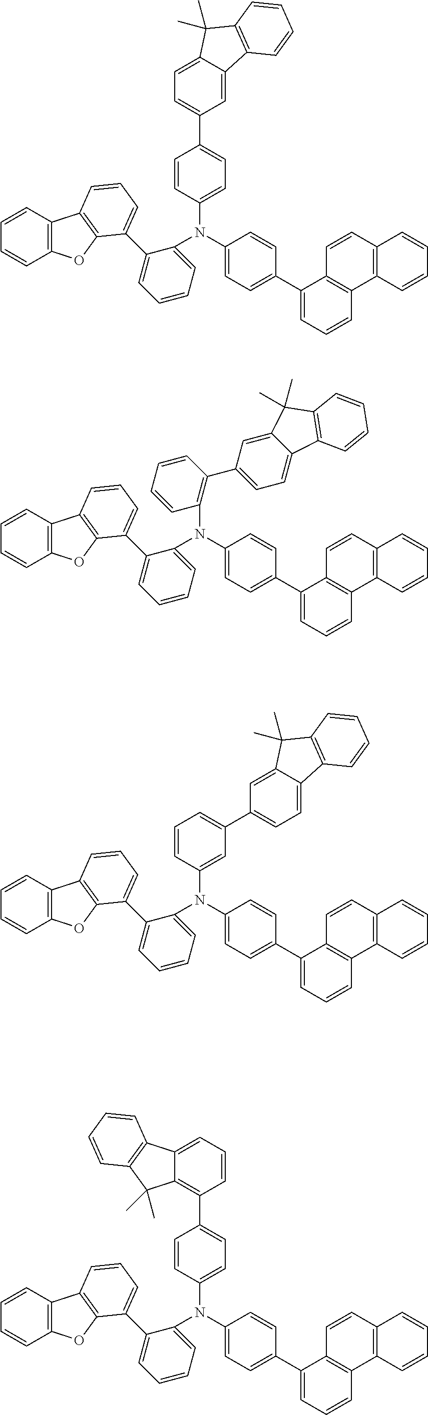 [SMILES: CC1(C)c2ccccc2-c2cc(-c3ccc(N(c4ccc(-c5cccc6c5ccc5ccccc56)cc4)c4ccccc4-c4cccc5c4oc4ccccc45)cc3)ccc21 ; CC1(C)c2ccccc2-c2ccc(-c3ccccc3N(c3ccc(-c4cccc5c4ccc4ccccc45)cc3)c3ccccc3-c3cccc4c3oc3ccccc34)cc21 ; CC1(C)c2ccccc2-c2ccc(-c3cccc(N(c4ccc(-c5cccc6c5ccc5ccccc56)cc4)c4ccccc4-c4cccc5c4oc4ccccc45)c3)cc21 ; CC1(C)c2ccccc2-c2cccc(-c3ccc(N(c4ccc(-c5cccc6c5ccc5ccccc56)cc4)c4ccccc4-c4cccc5c4oc4ccccc45)cc3)c21]

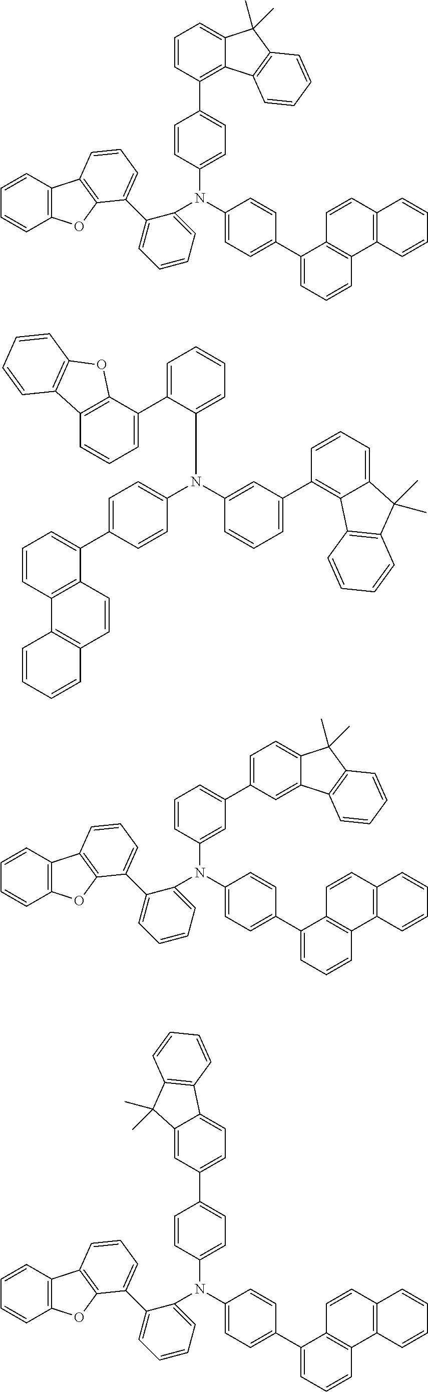 [SMILES: CC1(C)c2ccccc2-c2c(-c3ccc(N(c4ccc(-c5cccc6c5ccc5ccccc56)cc4)c4ccccc4-c4cccc5c4oc4ccccc45)cc3)cccc21 ; CC1(C)c2ccccc2-c2c(-c3cccc(N(c4ccc(-c5cccc6c5ccc5ccccc56)cc4)c4ccccc4-c4cccc5c4oc4ccccc45)c3)cccc21 ; CC1(C)c2ccccc2-c2cc(-c3cccc(N(c4ccc(-c5cccc6c5ccc5ccccc56)cc4)c4ccccc4-c4cccc5c4oc4ccccc45)c3)ccc21 ; CC1(C)c2ccccc2-c2ccc(-c3ccc(N(c4ccc(-c5cccc6c5ccc5ccccc56)cc4)c4ccccc4-c4cccc5c4oc4ccccc45)cc3)cc21]

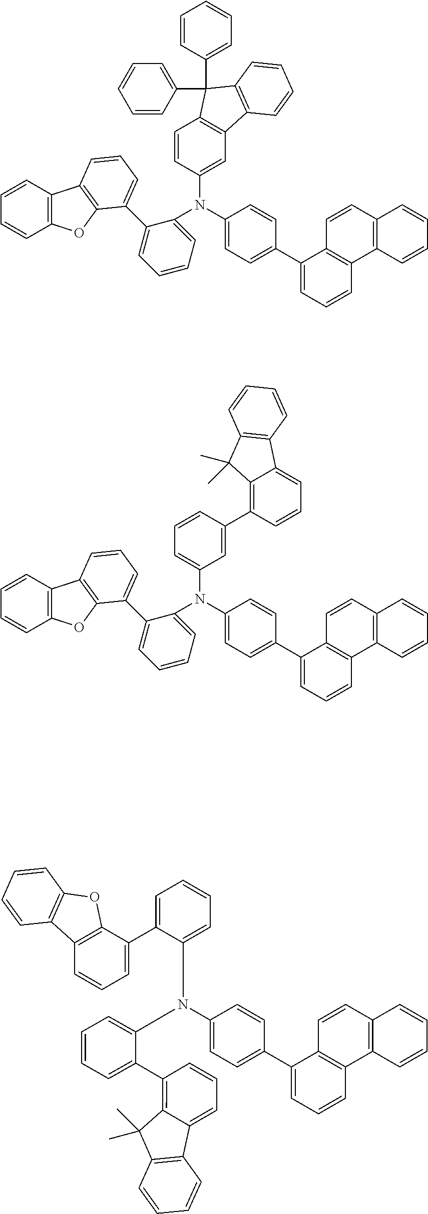 [SMILES: c1ccc(C2(c3ccccc3)c3ccccc3-c3cc(N(c4ccc(-c5cccc6c5ccc5ccccc56)cc4)c4ccccc4-c4cccc5c4oc4ccccc45)ccc32)cc1 ; CC1(C)c2ccccc2-c2cccc(-c3cccc(N(c4ccc(-c5cccc6c5ccc5ccccc56)cc4)c4ccccc4-c4cccc5c4oc4ccccc45)c3)c21 ; CC1(C)c2ccccc2-c2cccc(-c3ccccc3N(c3ccc(-c4cccc5c4ccc4ccccc45)cc3)c3ccccc3-c3cccc4c3oc3ccccc34)c21]

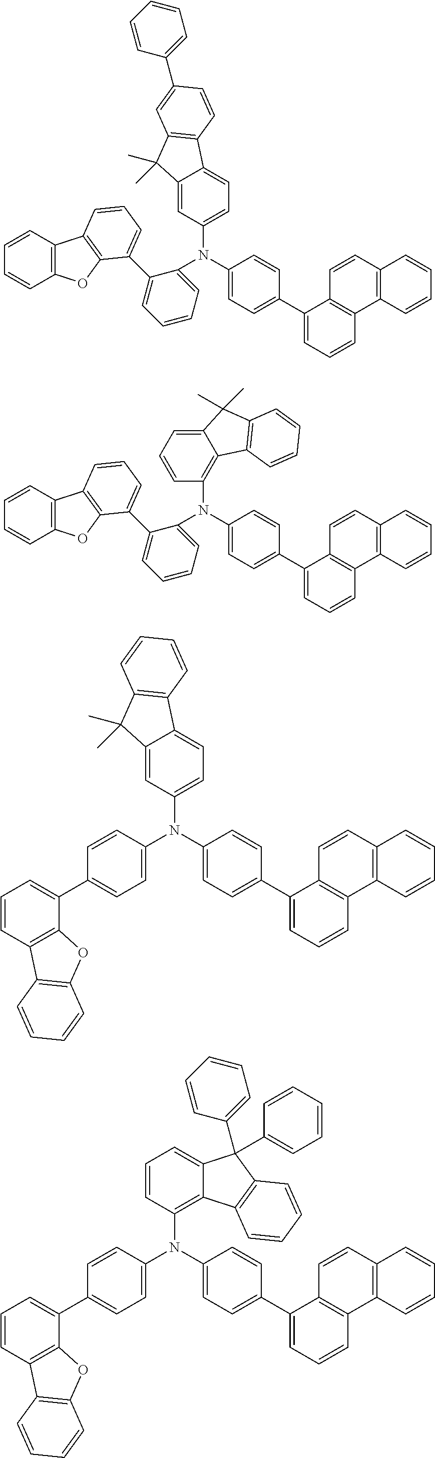 [SMILES: CC1(C)c2cc(-c3ccccc3)ccc2-c2ccc(N(c3ccc(-c4cccc5c4ccc4ccccc45)cc3)c3ccccc3-c3cccc4c3oc3ccccc34)cc21 ; CC1(C)c2ccccc2-c2c(N(c3ccc(-c4cccc5c4ccc4ccccc45)cc3)c3ccccc3-c3cccc4c3oc3ccccc34)cccc21 ; CC1(C)c2ccccc2-c2ccc(N(c3ccc(-c4cccc5c4ccc4ccccc45)cc3)c3ccc(-c4cccc5c4oc4ccccc45)cc3)cc21 ; c1ccc(C2(c3ccccc3)c3ccccc3-c3c(N(c4ccc(-c5cccc6c5ccc5ccccc56)cc4)c4ccc(-c5cccc6c5oc5ccccc56)cc4)cccc32)cc1]

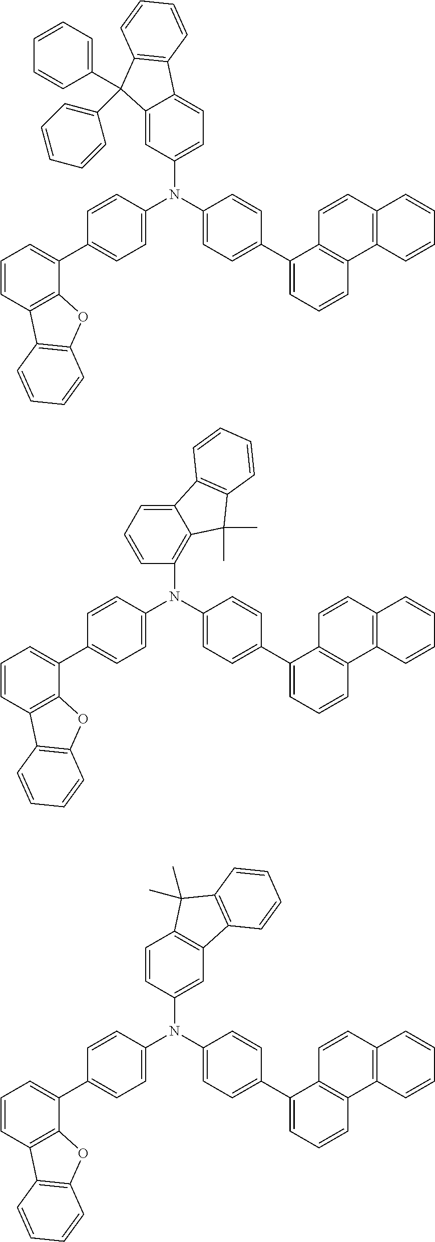 [SMILES: c1ccc(C2(c3ccccc3)c3ccccc3-c3ccc(N(c4ccc(-c5cccc6c5ccc5ccccc56)cc4)c4ccc(-c5cccc6c5oc5ccccc56)cc4)cc32)cc1 ; CC1(C)c2ccccc2-c2cccc(N(c3ccc(-c4cccc5c4ccc4ccccc45)cc3)c3ccc(-c4cccc5c4oc4ccccc45)cc3)c21 ; CC1(C)c2ccccc2-c2cc(N(c3ccc(-c4cccc5c4ccc4ccccc45)cc3)c3ccc(-c4cccc5c4oc4ccccc45)cc3)ccc21]

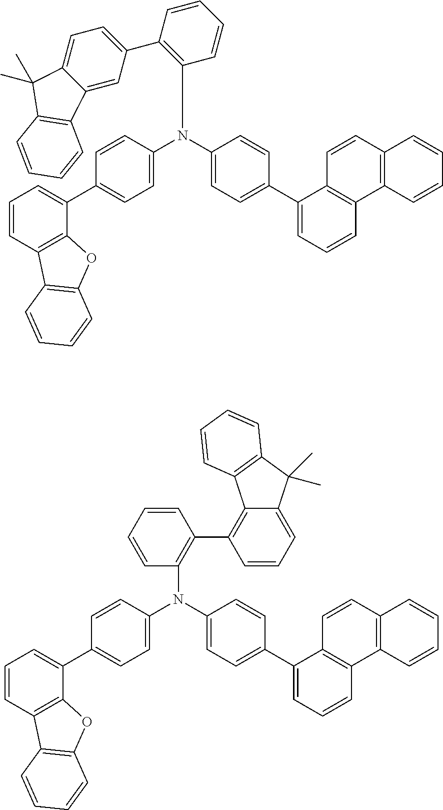 [SMILES: CC1(C)c2ccccc2-c2cc(-c3ccccc3N(c3ccc(-c4cccc5c4ccc4ccccc45)cc3)c3ccc(-c4cccc5c4oc4ccccc45)cc3)ccc21 ; CC1(C)c2ccccc2-c2c(-c3ccccc3N(c3ccc(-c4cccc5c4ccc4ccccc45)cc3)c3ccc(-c4cccc5c4oc4ccccc45)cc3)cccc21]

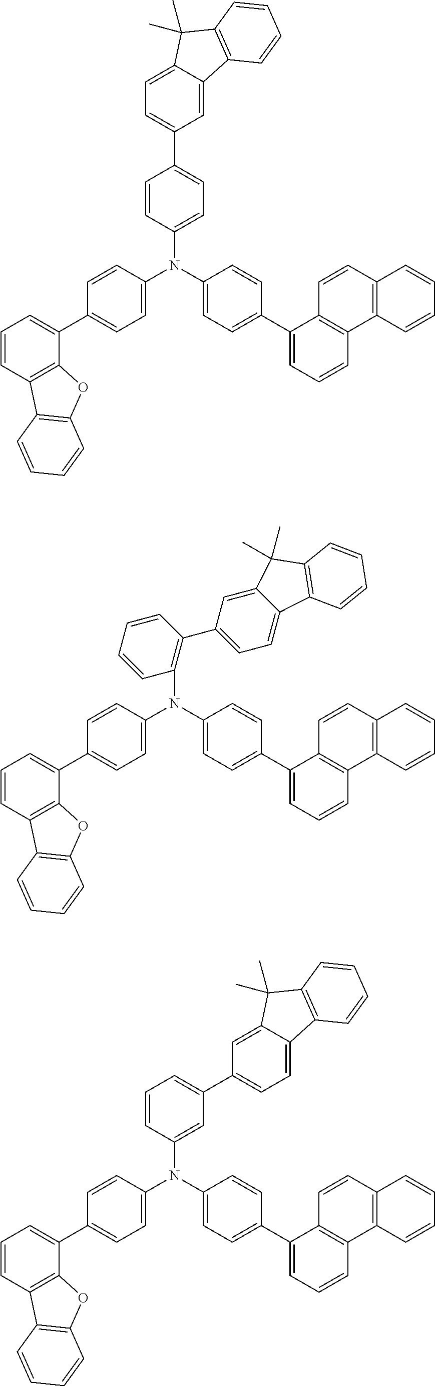 [SMILES: CC1(C)c2ccccc2-c2cc(-c3ccc(N(c4ccc(-c5cccc6c5ccc5ccccc56)cc4)c4ccc(-c5cccc6c5oc5ccccc56)cc4)cc3)ccc21 ; CC1(C)c2ccccc2-c2ccc(-c3ccccc3N(c3ccc(-c4cccc5c4ccc4ccccc45)cc3)c3ccc(-c4cccc5c4oc4ccccc45)cc3)cc21 ; CC1(C)c2ccccc2-c2ccc(-c3cccc(N(c4ccc(-c5cccc6c5ccc5ccccc56)cc4)c4ccc(-c5cccc6c5oc5ccccc56)cc4)c3)cc21]

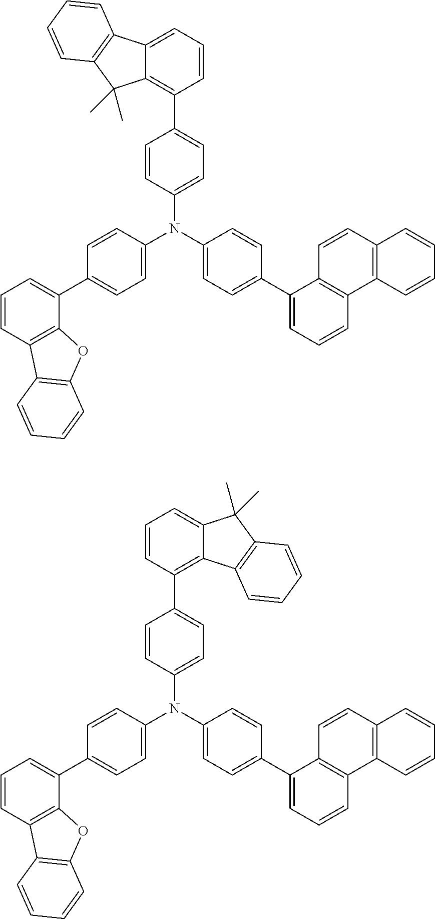 [SMILES: CC1(C)c2ccccc2-c2cccc(-c3ccc(N(c4ccc(-c5cccc6c5ccc5ccccc56)cc4)c4ccc(-c5cccc6c5oc5ccccc56)cc4)cc3)c21 ; CC1(C)c2ccccc2-c2c(-c3ccc(N(c4ccc(-c5cccc6c5ccc5ccccc56)cc4)c4ccc(-c5cccc6c5oc5ccccc56)cc4)cc3)cccc21]

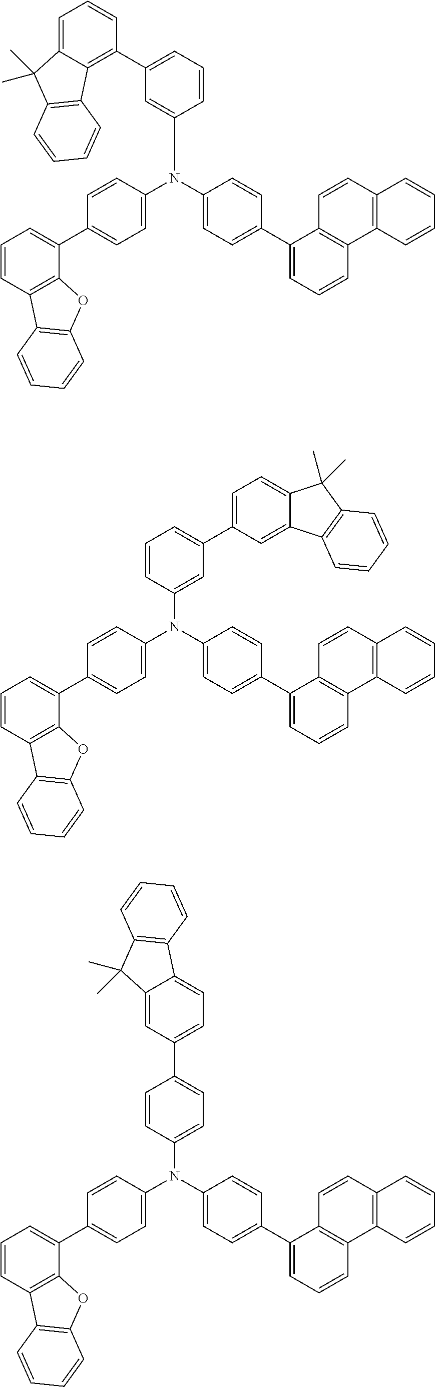 [SMILES: CC1(C)c2ccccc2-c2c(-c3cccc(N(c4ccc(-c5cccc6c5ccc5ccccc56)cc4)c4ccc(-c5cccc6c5oc5ccccc56)cc4)c3)cccc21 ; CC1(C)c2ccccc2-c2cc(-c3cccc(N(c4ccc(-c5cccc6c5ccc5ccccc56)cc4)c4ccc(-c5cccc6c5oc5ccccc56)cc4)c3)ccc21 ; CC1(C)c2ccccc2-c2ccc(-c3ccc(N(c4ccc(-c5cccc6c5ccc5ccccc56)cc4)c4ccc(-c5cccc6c5oc5ccccc56)cc4)cc3)cc21]

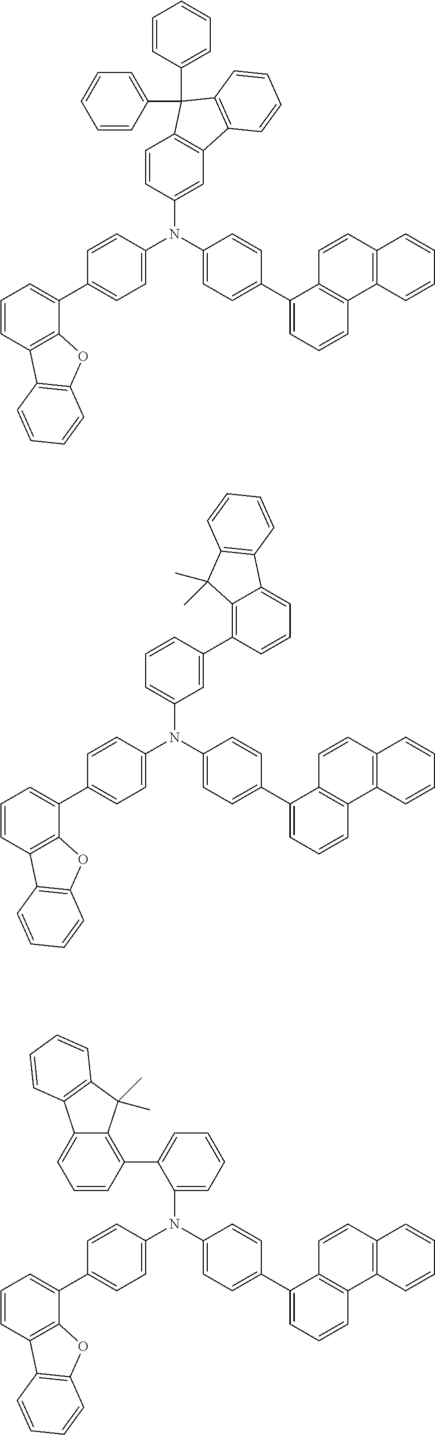 [SMILES: c1ccc(C2(c3ccccc3)c3ccccc3-c3cc(N(c4ccc(-c5cccc6c5ccc5ccccc56)cc4)c4ccc(-c5cccc6c5oc5ccccc56)cc4)ccc32)cc1 ; CC1(C)c2ccccc2-c2cccc(-c3cccc(N(c4ccc(-c5cccc6c5ccc5ccccc56)cc4)c4ccc(-c5cccc6c5oc5ccccc56)cc4)c3)c21 ; CC1(C)c2ccccc2-c2cccc(-c3ccccc3N(c3ccc(-c4cccc5c4ccc4ccccc45)cc3)c3ccc(-c4cccc5c4oc4ccccc45)cc3)c21]

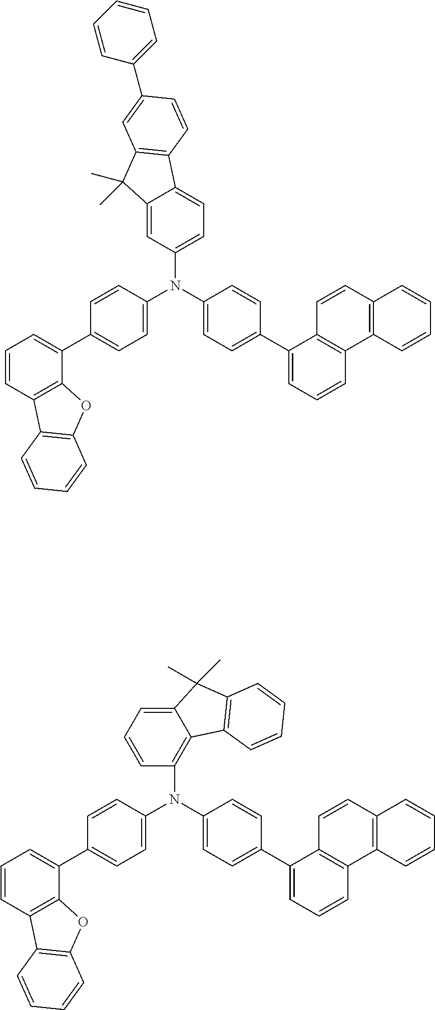 [SMILES: CC1(C)c2cc(-c3ccccc3)ccc2-c2ccc(N(c3ccc(-c4cccc5c4ccc4ccccc45)cc3)c3ccc(-c4cccc5c4oc4ccccc45)cc3)cc21 ; CC1(C)c2ccccc2-c2c(N(c3ccc(-c4cccc5c4ccc4ccccc45)cc3)c3ccc(-c4cccc5c4oc4ccccc45)cc3)cccc21]

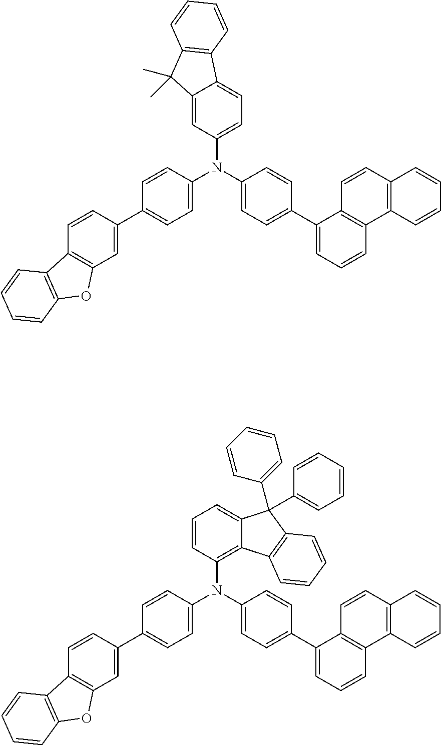 [SMILES: CC1(C)c2ccccc2-c2ccc(N(c3ccc(-c4ccc5c(c4)oc4ccccc45)cc3)c3ccc(-c4cccc5c4ccc4ccccc45)cc3)cc21 ; c1ccc(C2(c3ccccc3)c3ccccc3-c3c(N(c4ccc(-c5ccc6c(c5)oc5ccccc56)cc4)c4ccc(-c5cccc6c5ccc5ccccc56)cc4)cccc32)cc1]

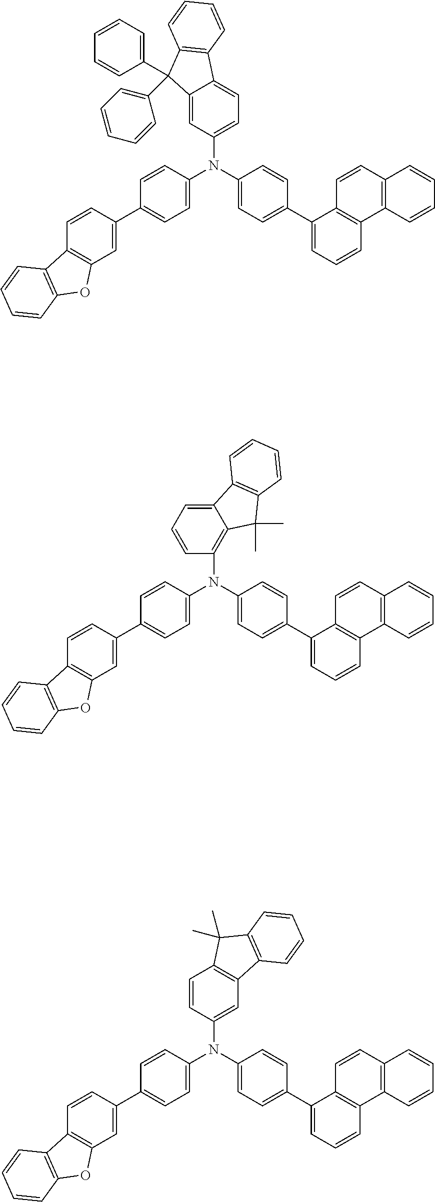 [SMILES: c1ccc(C2(c3ccccc3)c3ccccc3-c3ccc(N(c4ccc(-c5ccc6c(c5)oc5ccccc56)cc4)c4ccc(-c5cccc6c5ccc5ccccc56)cc4)cc32)cc1 ; CC1(C)c2ccccc2-c2cccc(N(c3ccc(-c4ccc5c(c4)oc4ccccc45)cc3)c3ccc(-c4cccc5c4ccc4ccccc45)cc3)c21 ; CC1(C)c2ccccc2-c2cc(N(c3ccc(-c4ccc5c(c4)oc4ccccc45)cc3)c3ccc(-c4cccc5c4ccc4ccccc45)cc3)ccc21]

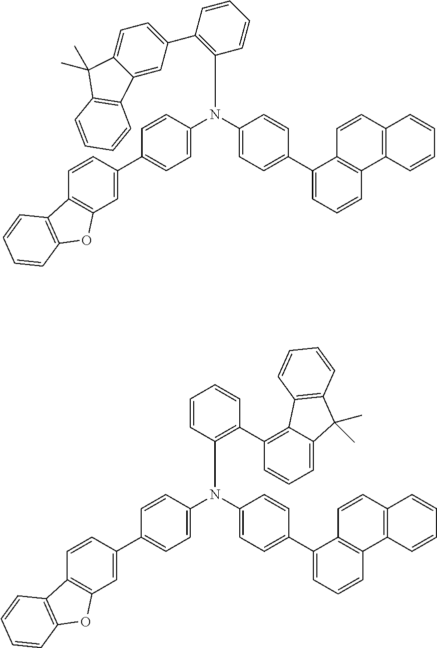 [SMILES: CC1(C)c2ccccc2-c2cc(-c3ccccc3N(c3ccc(-c4ccc5c(c4)oc4ccccc45)cc3)c3ccc(-c4cccc5c4ccc4ccccc45)cc3)ccc21 ; CC1(C)c2ccccc2-c2c(-c3ccccc3N(c3ccc(-c4ccc5c(c4)oc4ccccc45)cc3)c3ccc(-c4cccc5c4ccc4ccccc45)cc3)cccc21]

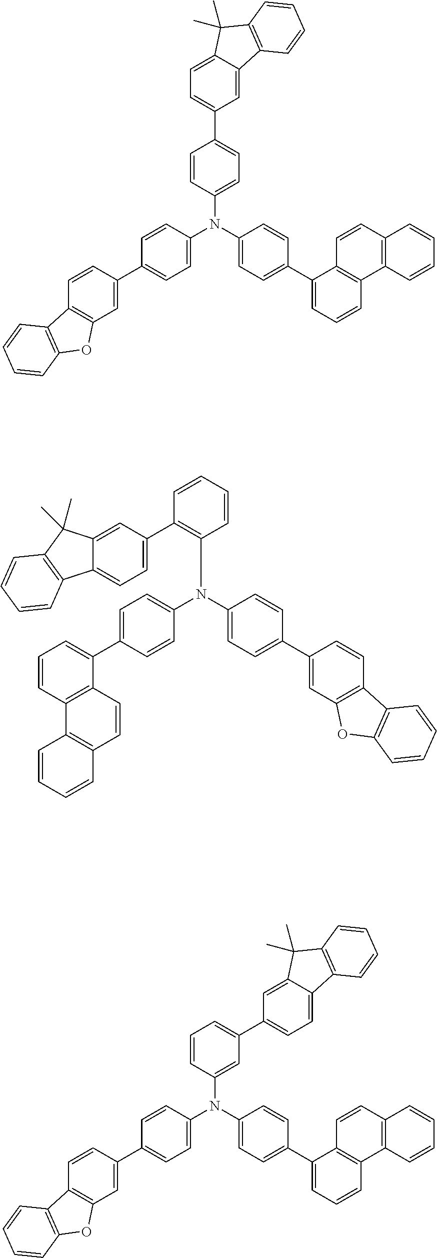 [SMILES: CC1(C)c2ccccc2-c2cc(-c3ccc(N(c4ccc(-c5ccc6c(c5)oc5ccccc56)cc4)c4ccc(-c5cccc6c5ccc5ccccc56)cc4)cc3)ccc21 ; CC1(C)c2ccccc2-c2ccc(-c3ccccc3N(c3ccc(-c4ccc5c(c4)oc4ccccc45)cc3)c3ccc(-c4cccc5c4ccc4ccccc45)cc3)cc21 ; CC1(C)c2ccccc2-c2ccc(-c3cccc(N(c4ccc(-c5ccc6c(c5)oc5ccccc56)cc4)c4ccc(-c5cccc6c5ccc5ccccc56)cc4)c3)cc21]

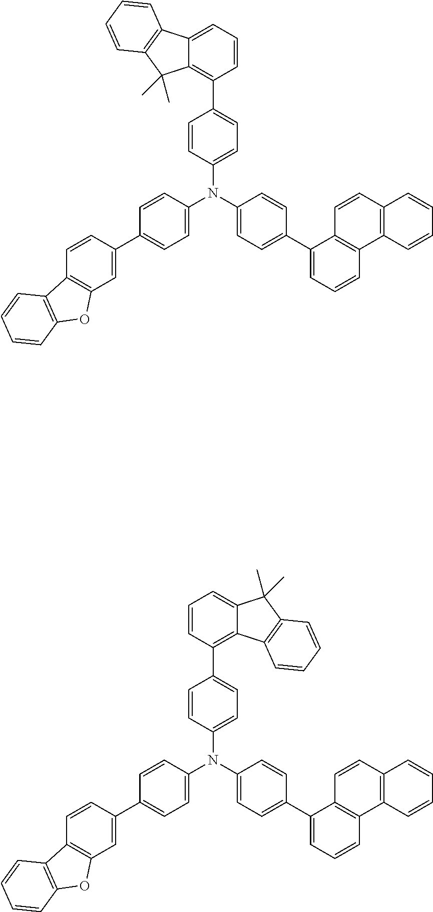 [SMILES: CC1(C)c2ccccc2-c2cccc(-c3ccc(N(c4ccc(-c5ccc6c(c5)oc5ccccc56)cc4)c4ccc(-c5cccc6c5ccc5ccccc56)cc4)cc3)c21 ; CC1(C)c2ccccc2-c2c(-c3ccc(N(c4ccc(-c5ccc6c(c5)oc5ccccc56)cc4)c4ccc(-c5cccc6c5ccc5ccccc56)cc4)cc3)cccc21]

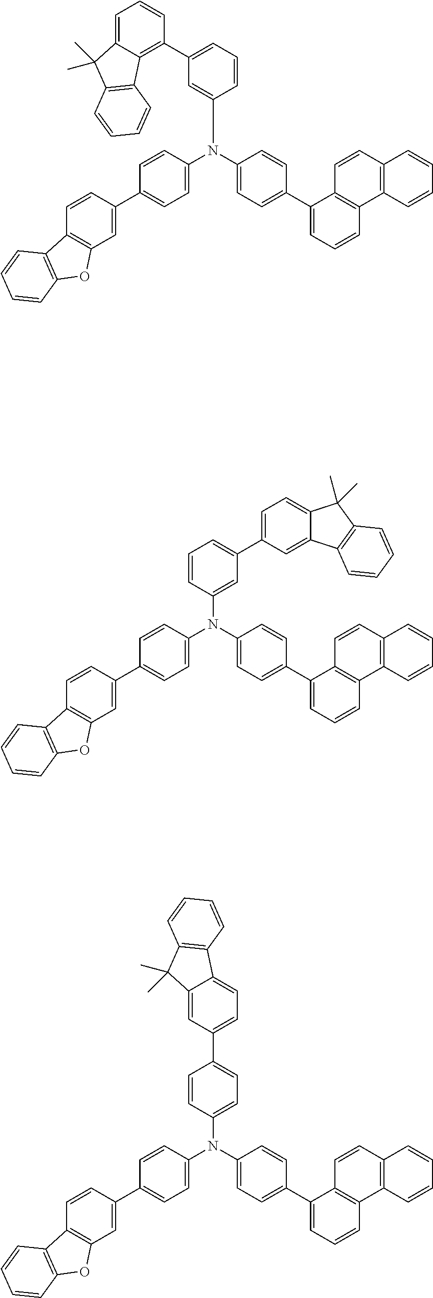 [SMILES: CC1(C)c2ccccc2-c2c(-c3cccc(N(c4ccc(-c5ccc6c(c5)oc5ccccc56)cc4)c4ccc(-c5cccc6c5ccc5ccccc56)cc4)c3)cccc21 ; CC1(C)c2ccccc2-c2cc(-c3cccc(N(c4ccc(-c5ccc6c(c5)oc5ccccc56)cc4)c4ccc(-c5cccc6c5ccc5ccccc56)cc4)c3)ccc21 ; CC1(C)c2ccccc2-c2ccc(-c3ccc(N(c4ccc(-c5ccc6c(c5)oc5ccccc56)cc4)c4ccc(-c5cccc6c5ccc5ccccc56)cc4)cc3)cc21]

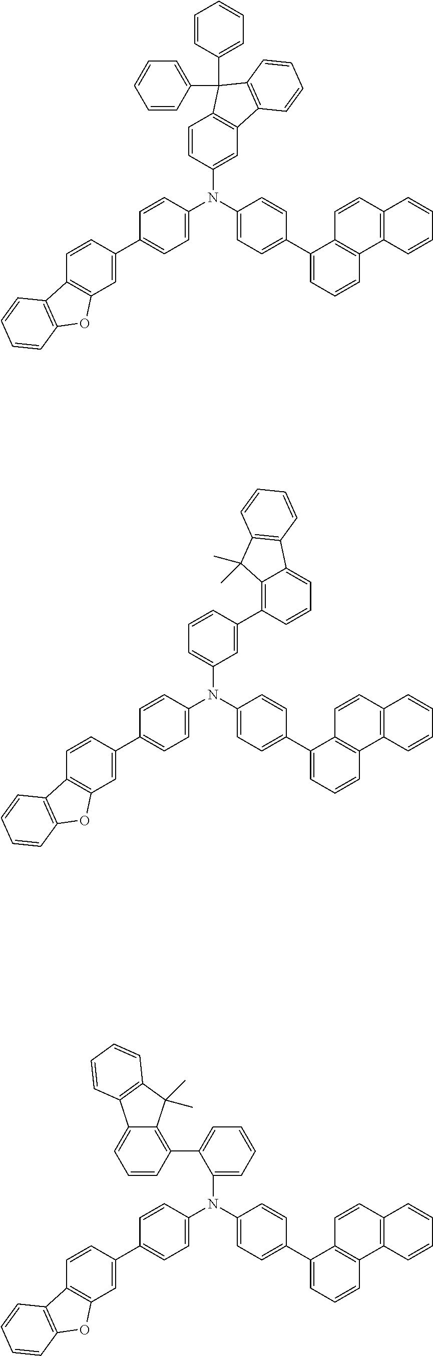 [SMILES: c1ccc(C2(c3ccccc3)c3ccccc3-c3cc(N(c4ccc(-c5ccc6c(c5)oc5ccccc56)cc4)c4ccc(-c5cccc6c5ccc5ccccc56)cc4)ccc32)cc1 ; CC1(C)c2ccccc2-c2cccc(-c3cccc(N(c4ccc(-c5ccc6c(c5)oc5ccccc56)cc4)c4ccc(-c5cccc6c5ccc5ccccc56)cc4)c3)c21 ; CC1(C)c2ccccc2-c2cccc(-c3ccccc3N(c3ccc(-c4ccc5c(c4)oc4ccccc45)cc3)c3ccc(-c4cccc5c4ccc4ccccc45)cc3)c21]

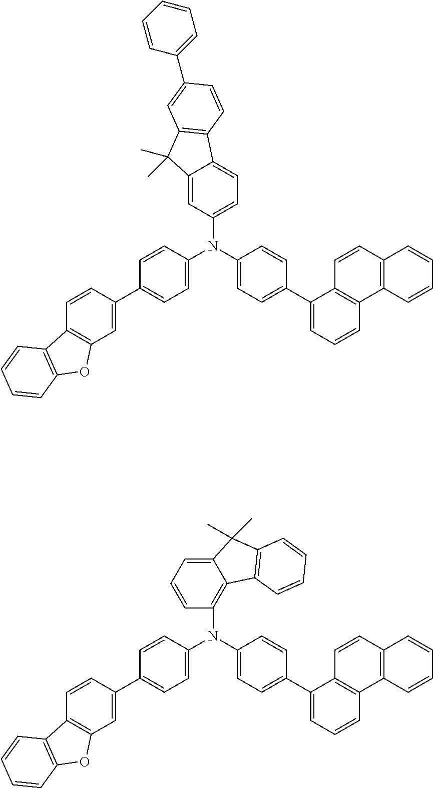 [SMILES: CC1(C)c2cc(-c3ccccc3)ccc2-c2ccc(N(c3ccc(-c4ccc5c(c4)oc4ccccc45)cc3)c3ccc(-c4cccc5c4ccc4ccccc45)cc3)cc21 ; CC1(C)c2ccccc2-c2c(N(c3ccc(-c4ccc5c(c4)oc4ccccc45)cc3)c3ccc(-c4cccc5c4ccc4ccccc45)cc3)cccc21]

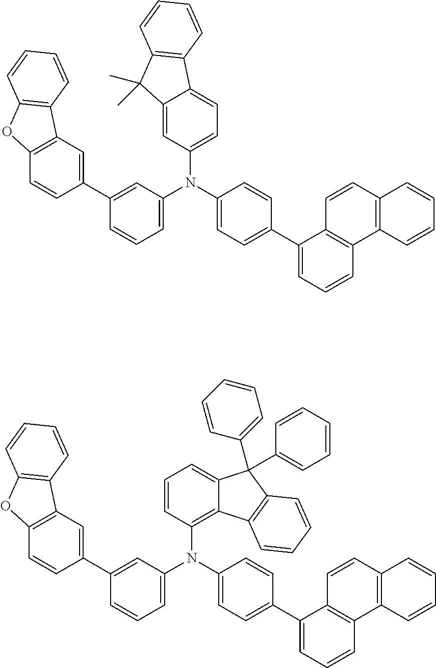 [SMILES: CC1(C)c2ccccc2-c2ccc(N(c3ccc(-c4cccc5c4ccc4ccccc45)cc3)c3cccc(-c4ccc5oc6ccccc6c5c4)c3)cc21 ; c1ccc(C2(c3ccccc3)c3ccccc3-c3c(N(c4ccc(-c5cccc6c5ccc5ccccc56)cc4)c4cccc(-c5ccc6oc7ccccc7c6c5)c4)cccc32)cc1]

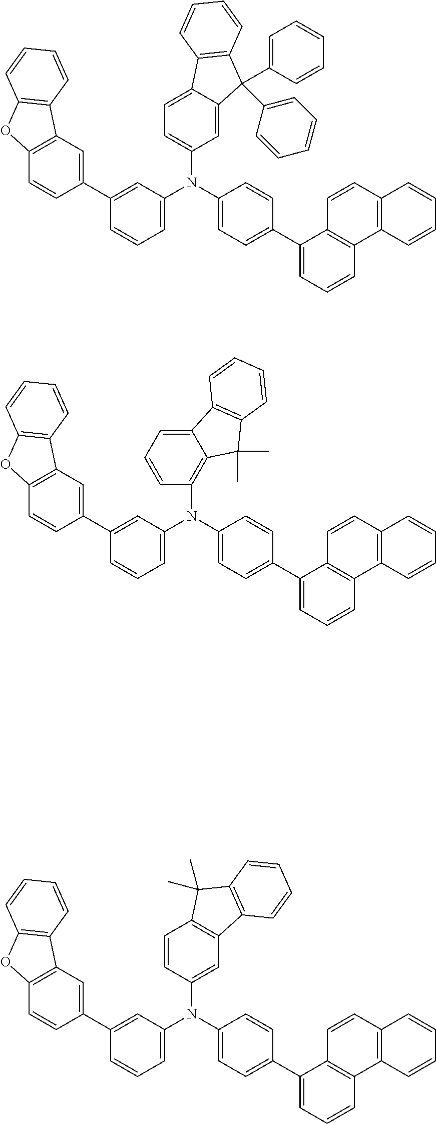 [SMILES: c1ccc(C2(c3ccccc3)c3ccccc3-c3ccc(N(c4ccc(-c5cccc6c5ccc5ccccc56)cc4)c4cccc(-c5ccc6oc7ccccc7c6c5)c4)cc32)cc1 ; CC1(C)c2ccccc2-c2cccc(N(c3ccc(-c4cccc5c4ccc4ccccc45)cc3)c3cccc(-c4ccc5oc6ccccc6c5c4)c3)c21 ; CC1(C)c2ccccc2-c2cc(N(c3ccc(-c4cccc5c4ccc4ccccc45)cc3)c3cccc(-c4ccc5oc6ccccc6c5c4)c3)ccc21]

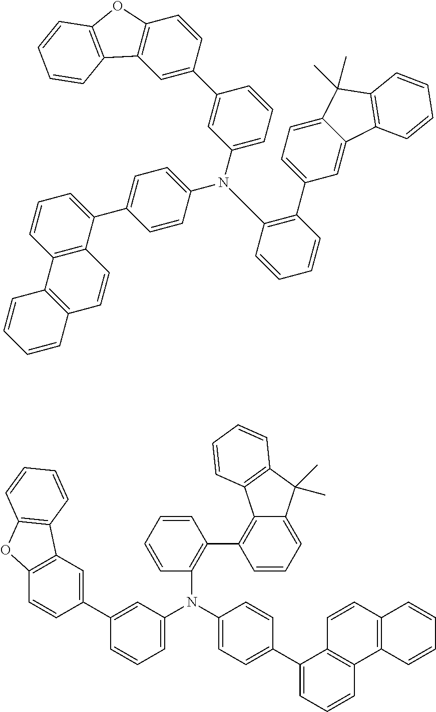 [SMILES: CC1(C)c2ccccc2-c2cc(-c3ccccc3N(c3ccc(-c4cccc5c4ccc4ccccc45)cc3)c3cccc(-c4ccc5oc6ccccc6c5c4)c3)ccc21 ; CC1(C)c2ccccc2-c2c(-c3ccccc3N(c3ccc(-c4cccc5c4ccc4ccccc45)cc3)c3cccc(-c4ccc5oc6ccccc6c5c4)c3)cccc21]

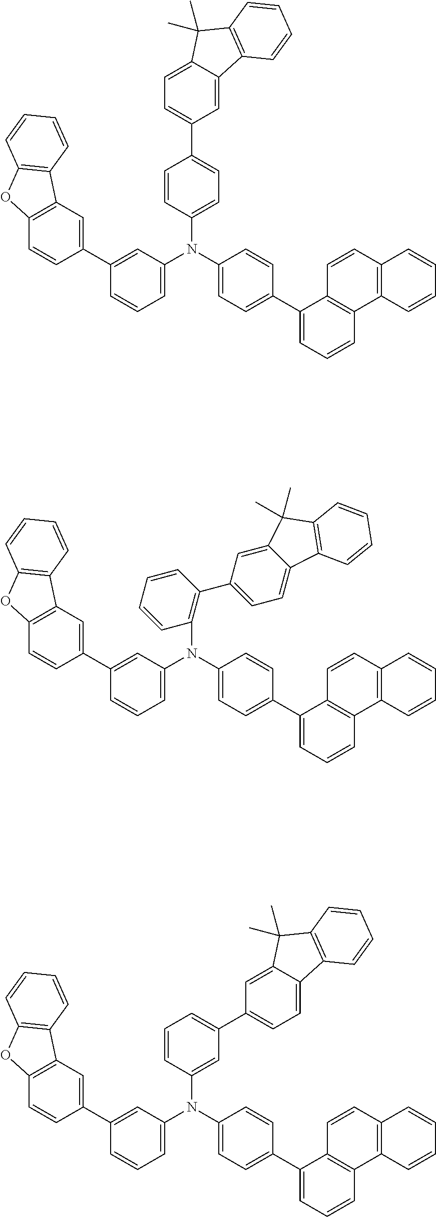 [SMILES: CC1(C)c2ccccc2-c2cc(-c3ccc(N(c4ccc(-c5cccc6c5ccc5ccccc56)cc4)c4cccc(-c5ccc6oc7ccccc7c6c5)c4)cc3)ccc21 ; CC1(C)c2ccccc2-c2ccc(-c3ccccc3N(c3ccc(-c4cccc5c4ccc4ccccc45)cc3)c3cccc(-c4ccc5oc6ccccc6c5c4)c3)cc21 ; CC1(C)c2ccccc2-c2ccc(-c3cccc(N(c4ccc(-c5cccc6c5ccc5ccccc56)cc4)c4cccc(-c5ccc6oc7ccccc7c6c5)c4)c3)cc21]

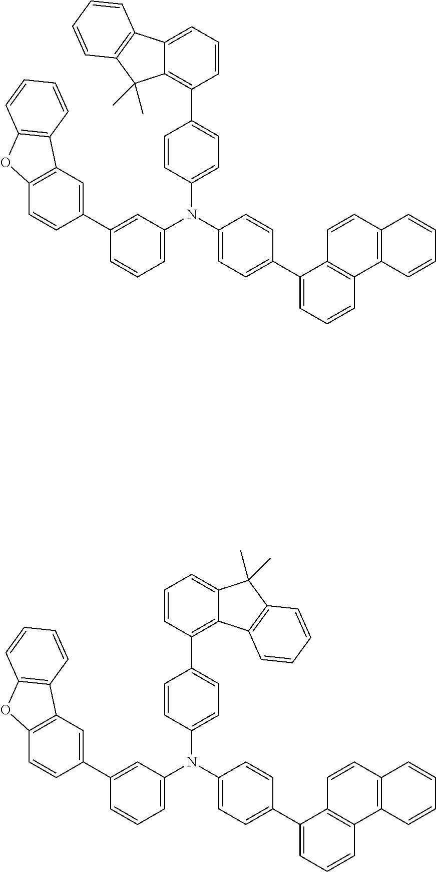 [SMILES: CC1(C)c2ccccc2-c2cccc(-c3ccc(N(c4ccc(-c5cccc6c5ccc5ccccc56)cc4)c4cccc(-c5ccc6oc7ccccc7c6c5)c4)cc3)c21 ; CC1(C)c2ccccc2-c2c(-c3ccc(N(c4ccc(-c5cccc6c5ccc5ccccc56)cc4)c4cccc(-c5ccc6oc7ccccc7c6c5)c4)cc3)cccc21]

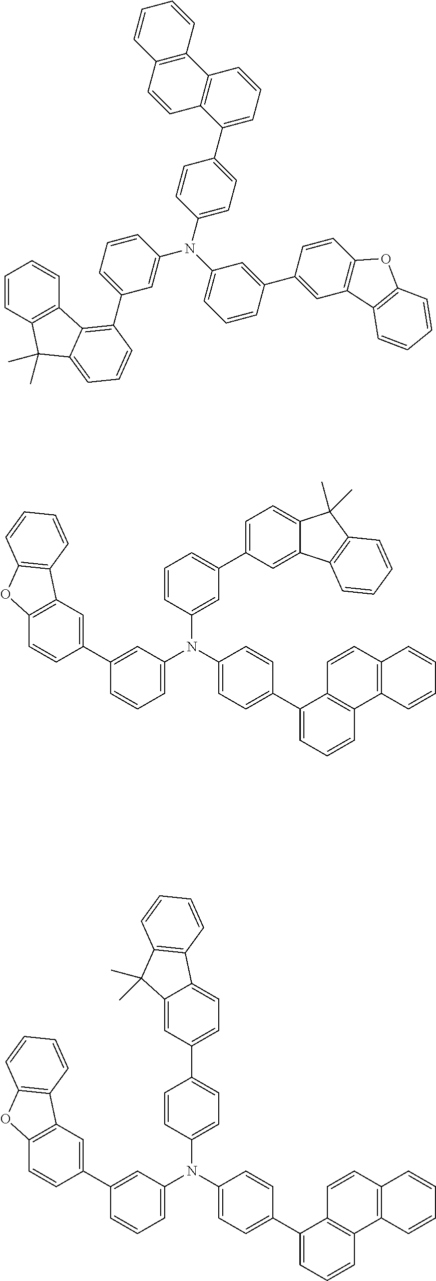 [SMILES: CC1(C)c2ccccc2-c2c(-c3cccc(N(c4ccc(-c5cccc6c5ccc5ccccc56)cc4)c4cccc(-c5ccc6oc7ccccc7c6c5)c4)c3)cccc21 ; CC1(C)c2ccccc2-c2cc(-c3cccc(N(c4ccc(-c5cccc6c5ccc5ccccc56)cc4)c4cccc(-c5ccc6oc7ccccc7c6c5)c4)c3)ccc21 ; CC1(C)c2ccccc2-c2ccc(-c3ccc(N(c4ccc(-c5cccc6c5ccc5ccccc56)cc4)c4cccc(-c5ccc6oc7ccccc7c6c5)c4)cc3)cc21]

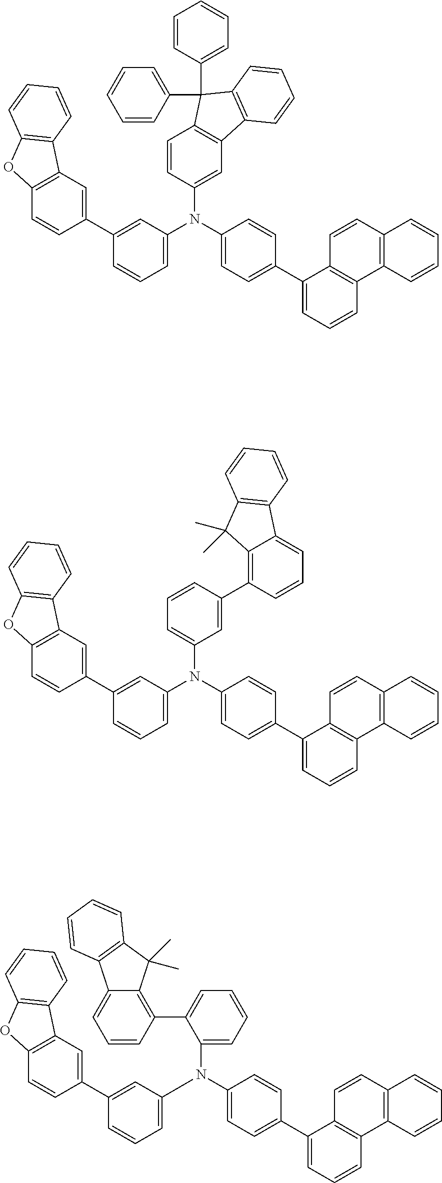 [SMILES: c1ccc(C2(c3ccccc3)c3ccccc3-c3cc(N(c4ccc(-c5cccc6c5ccc5ccccc56)cc4)c4cccc(-c5ccc6oc7ccccc7c6c5)c4)ccc32)cc1 ; CC1(C)c2ccccc2-c2cccc(-c3cccc(N(c4ccc(-c5cccc6c5ccc5ccccc56)cc4)c4cccc(-c5ccc6oc7ccccc7c6c5)c4)c3)c21 ; CC1(C)c2ccccc2-c2cccc(-c3ccccc3N(c3ccc(-c4cccc5c4ccc4ccccc45)cc3)c3cccc(-c4ccc5oc6ccccc6c5c4)c3)c21]

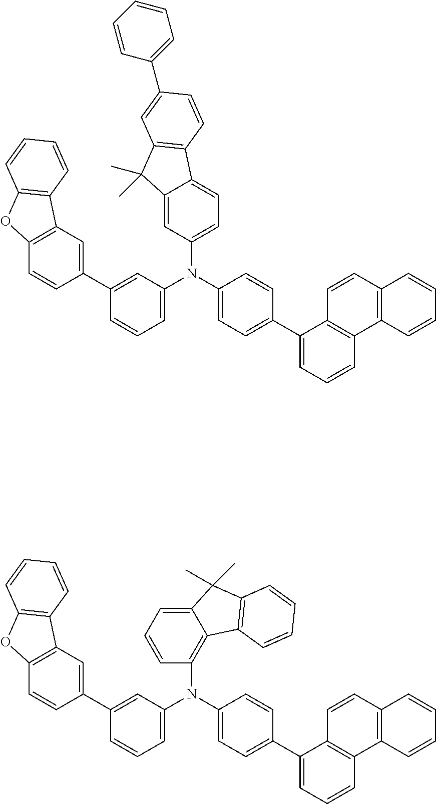 [SMILES: CC1(C)c2cc(-c3ccccc3)ccc2-c2ccc(N(c3ccc(-c4cccc5c4ccc4ccccc45)cc3)c3cccc(-c4ccc5oc6ccccc6c5c4)c3)cc21 ; CC1(C)c2ccccc2-c2c(N(c3ccc(-c4cccc5c4ccc4ccccc45)cc3)c3cccc(-c4ccc5oc6ccccc6c5c4)c3)cccc21]

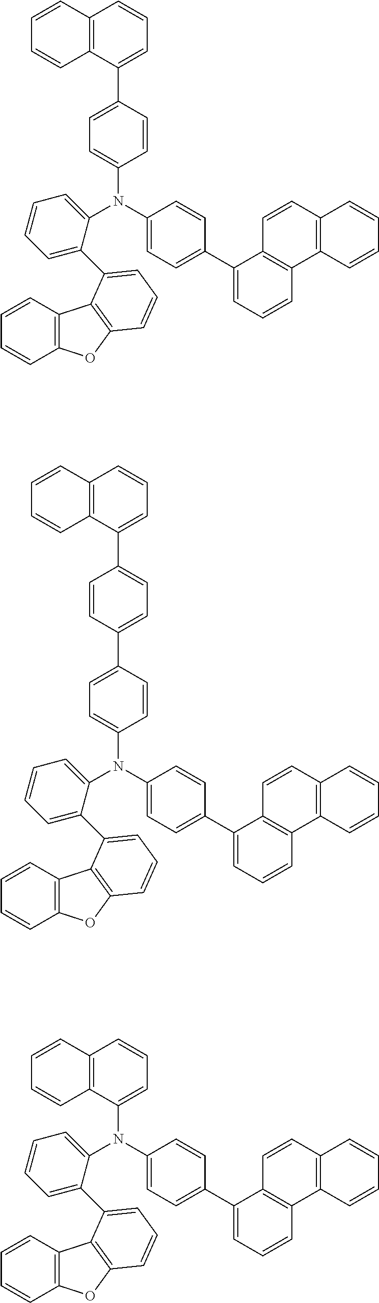 [SMILES: c1ccc(N(c2ccc(-c3cccc4ccccc34)cc2)c2ccc(-c3cccc4c3ccc3ccccc34)cc2)c(-c2cccc3oc4ccccc4c23)c1 ; c1ccc(N(c2ccc(-c3ccc(-c4cccc5ccccc45)cc3)cc2)c2ccc(-c3cccc4c3ccc3ccccc34)cc2)c(-c2cccc3oc4ccccc4c23)c1 ; c1ccc(N(c2ccc(-c3cccc4c3ccc3ccccc34)cc2)c2cccc3ccccc23)c(-c2cccc3oc4ccccc4c23)c1]

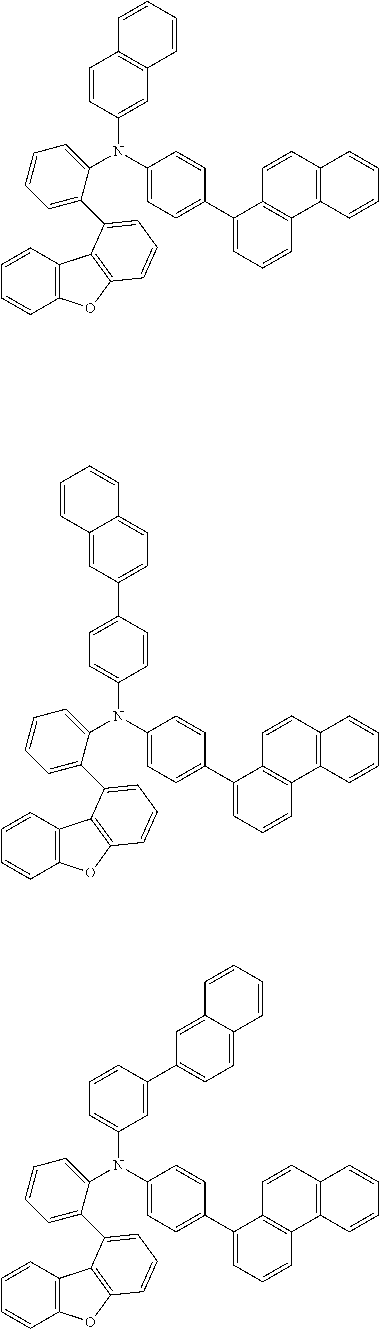 [SMILES: c1ccc(N(c2ccc(-c3cccc4c3ccc3ccccc34)cc2)c2ccc3ccccc3c2)c(-c2cccc3oc4ccccc4c23)c1 ; c1ccc(N(c2ccc(-c3ccc4ccccc4c3)cc2)c2ccc(-c3cccc4c3ccc3ccccc34)cc2)c(-c2cccc3oc4ccccc4c23)c1 ; c1cc(-c2ccc3ccccc3c2)cc(N(c2ccc(-c3cccc4c3ccc3ccccc34)cc2)c2ccccc2-c2cccc3oc4ccccc4c23)c1]

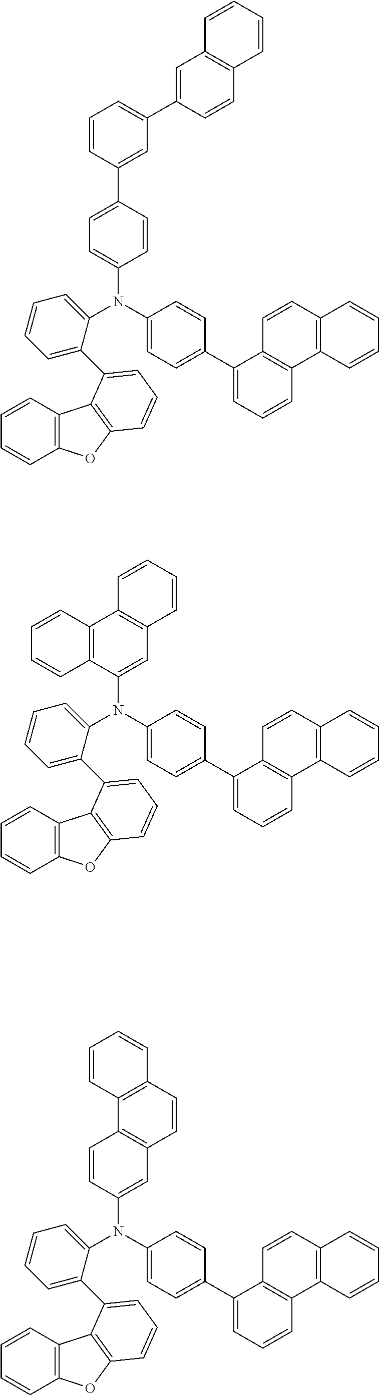 [SMILES: c1cc(-c2ccc(N(c3ccc(-c4cccc5c4ccc4ccccc45)cc3)c3ccccc3-c3cccc4oc5ccccc5c34)cc2)cc(-c2ccc3ccccc3c2)c1 ; c1ccc(N(c2ccc(-c3cccc4c3ccc3ccccc34)cc2)c2cc3ccccc3c3ccccc23)c(-c2cccc3oc4ccccc4c23)c1 ; c1ccc(N(c2ccc(-c3cccc4c3ccc3ccccc34)cc2)c2ccc3c(ccc4ccccc43)c2)c(-c2cccc3oc4ccccc4c23)c1]

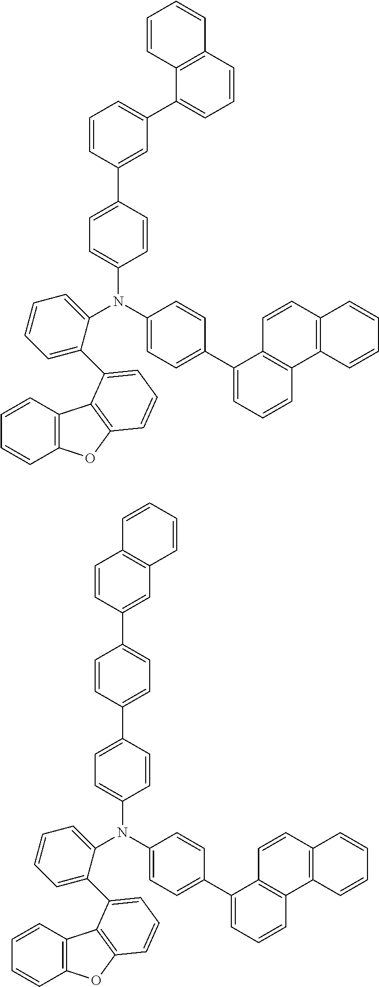 [SMILES: c1cc(-c2ccc(N(c3ccc(-c4cccc5c4ccc4ccccc45)cc3)c3ccccc3-c3cccc4oc5ccccc5c34)cc2)cc(-c2cccc3ccccc23)c1 ; c1ccc(N(c2ccc(-c3ccc(-c4ccc5ccccc5c4)cc3)cc2)c2ccc(-c3cccc4c3ccc3ccccc34)cc2)c(-c2cccc3oc4ccccc4c23)c1]

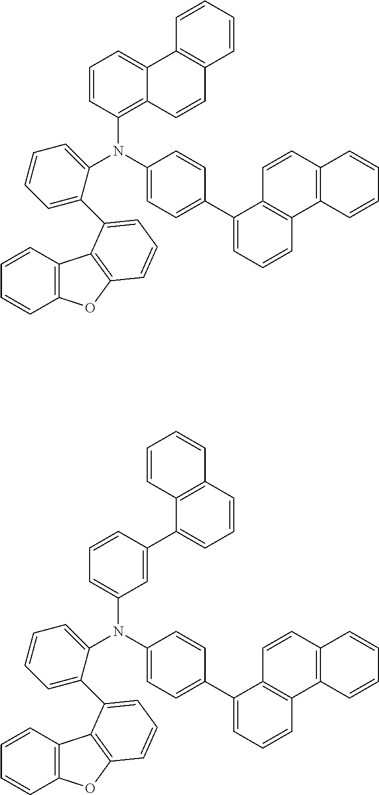 [SMILES: c1ccc(N(c2ccc(-c3cccc4c3ccc3ccccc34)cc2)c2cccc3c2ccc2ccccc23)c(-c2cccc3oc4ccccc4c23)c1 ; c1cc(-c2cccc3ccccc23)cc(N(c2ccc(-c3cccc4c3ccc3ccccc34)cc2)c2ccccc2-c2cccc3oc4ccccc4c23)c1]

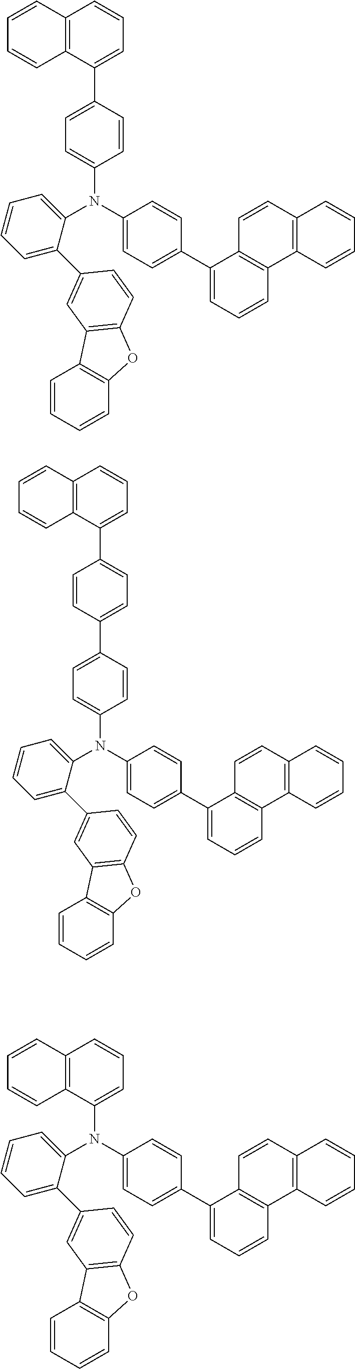

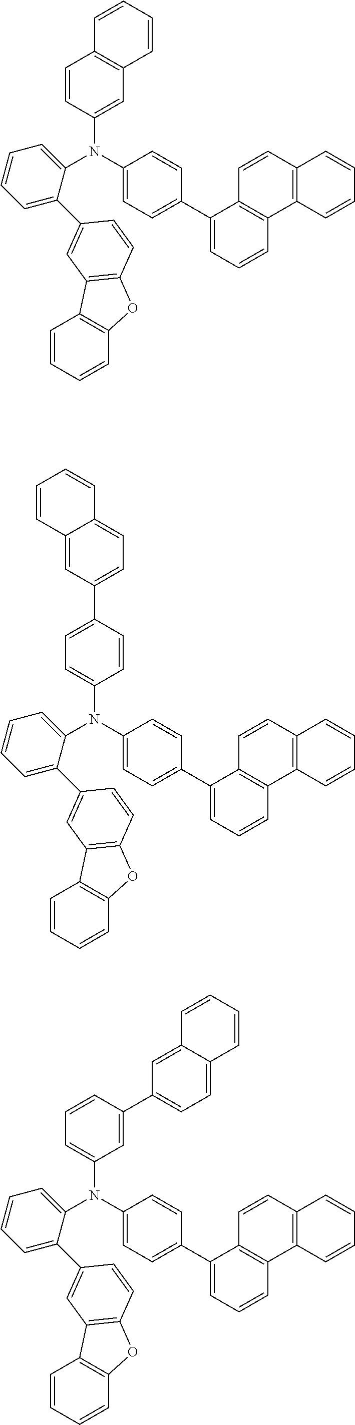 [SMILES: c1ccc(N(c2ccc(-c3cccc4c3ccc3ccccc34)cc2)c2ccc3ccccc3c2)c(-c2ccc3oc4ccccc4c3c2)c1 ; c1ccc(N(c2ccc(-c3ccc4ccccc4c3)cc2)c2ccc(-c3cccc4c3ccc3ccccc34)cc2)c(-c2ccc3oc4ccccc4c3c2)c1 ; c1cc(-c2ccc3ccccc3c2)cc(N(c2ccc(-c3cccc4c3ccc3ccccc34)cc2)c2ccccc2-c2ccc3oc4ccccc4c3c2)c1]

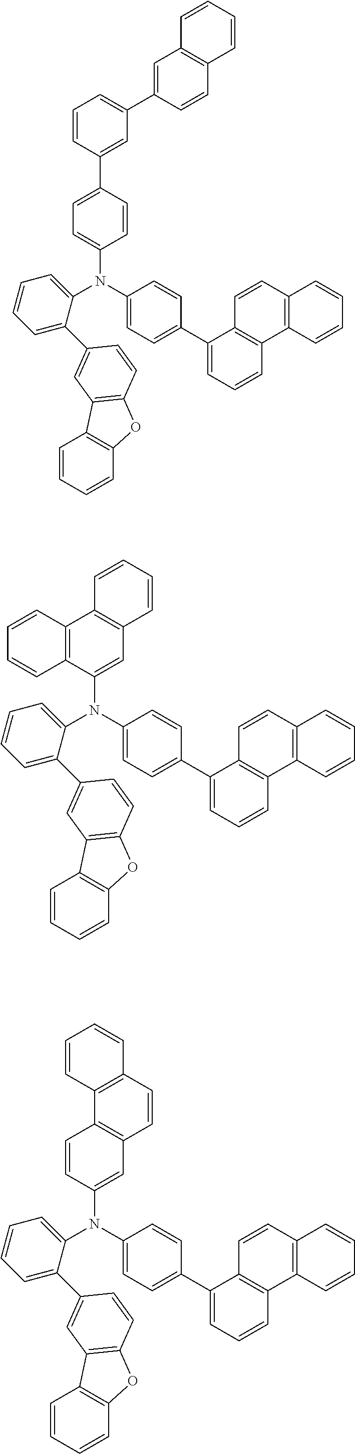 [SMILES: c1cc(-c2ccc(N(c3ccc(-c4cccc5c4ccc4ccccc45)cc3)c3ccccc3-c3ccc4oc5ccccc5c4c3)cc2)cc(-c2ccc3ccccc3c2)c1 ; c1ccc(N(c2ccc(-c3cccc4c3ccc3ccccc34)cc2)c2cc3ccccc3c3ccccc23)c(-c2ccc3oc4ccccc4c3c2)c1 ; c1ccc(N(c2ccc(-c3cccc4c3ccc3ccccc34)cc2)c2ccc3c(ccc4ccccc43)c2)c(-c2ccc3oc4ccccc4c3c2)c1]

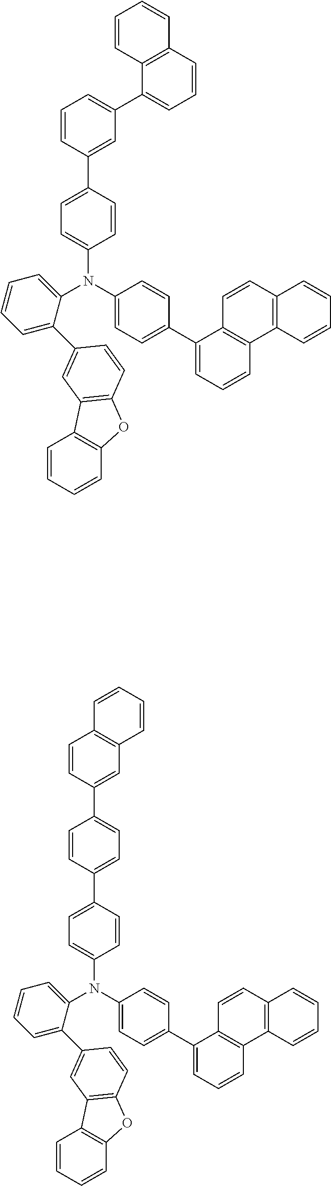 [SMILES: c1cc(-c2ccc(N(c3ccc(-c4cccc5c4ccc4ccccc45)cc3)c3ccccc3-c3ccc4oc5ccccc5c4c3)cc2)cc(-c2cccc3ccccc23)c1 ; c1ccc(N(c2ccc(-c3ccc(-c4ccc5ccccc5c4)cc3)cc2)c2ccc(-c3cccc4c3ccc3ccccc34)cc2)c(-c2ccc3oc4ccccc4c3c2)c1]

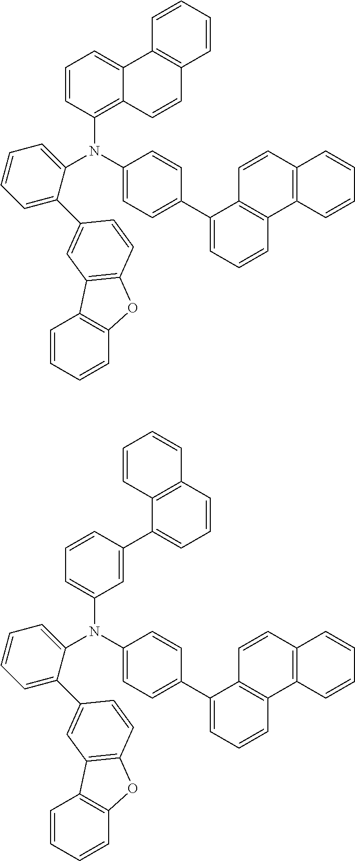 [SMILES: c1ccc(N(c2ccc(-c3cccc4c3ccc3ccccc34)cc2)c2cccc3c2ccc2ccccc23)c(-c2ccc3oc4ccccc4c3c2)c1 ; c1cc(-c2cccc3ccccc23)cc(N(c2ccc(-c3cccc4c3ccc3ccccc34)cc2)c2ccccc2-c2ccc3oc4ccccc4c3c2)c1]

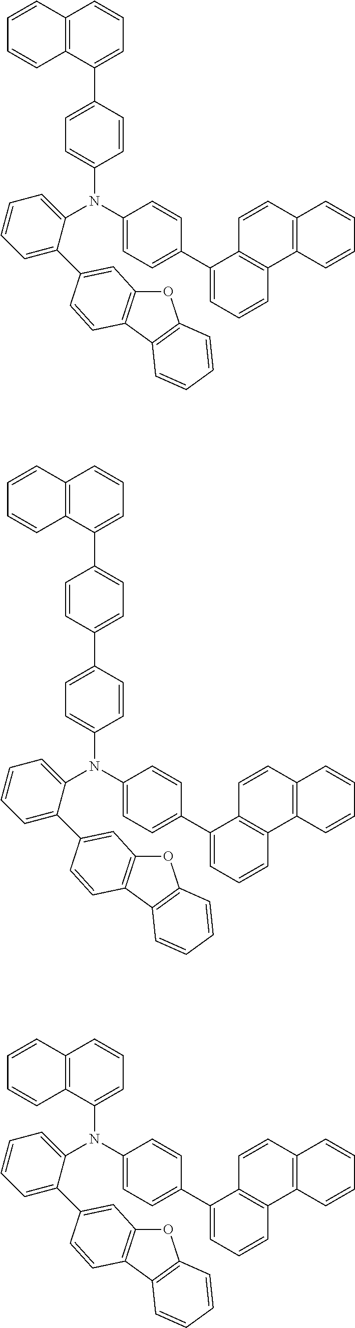 [SMILES: c1ccc(N(c2ccc(-c3cccc4ccccc34)cc2)c2ccc(-c3cccc4c3ccc3ccccc34)cc2)c(-c2ccc3c(c2)oc2ccccc23)c1 ; c1ccc(N(c2ccc(-c3ccc(-c4cccc5ccccc45)cc3)cc2)c2ccc(-c3cccc4c3ccc3ccccc34)cc2)c(-c2ccc3c(c2)oc2ccccc23)c1 ; c1ccc(N(c2ccc(-c3cccc4c3ccc3ccccc34)cc2)c2cccc3ccccc23)c(-c2ccc3c(c2)oc2ccccc23)c1]

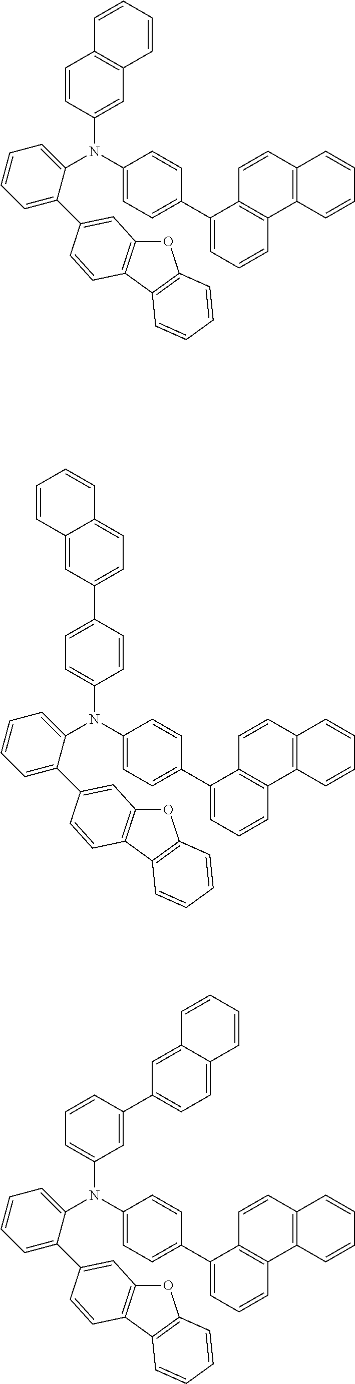 [SMILES: c1ccc(N(c2ccc(-c3cccc4c3ccc3ccccc34)cc2)c2ccc3ccccc3c2)c(-c2ccc3c(c2)oc2ccccc23)c1 ; c1ccc(N(c2ccc(-c3ccc4ccccc4c3)cc2)c2ccc(-c3cccc4c3ccc3ccccc34)cc2)c(-c2ccc3c(c2)oc2ccccc23)c1 ; c1cc(-c2ccc3ccccc3c2)cc(N(c2ccc(-c3cccc4c3ccc3ccccc34)cc2)c2ccccc2-c2ccc3c(c2)oc2ccccc23)c1]

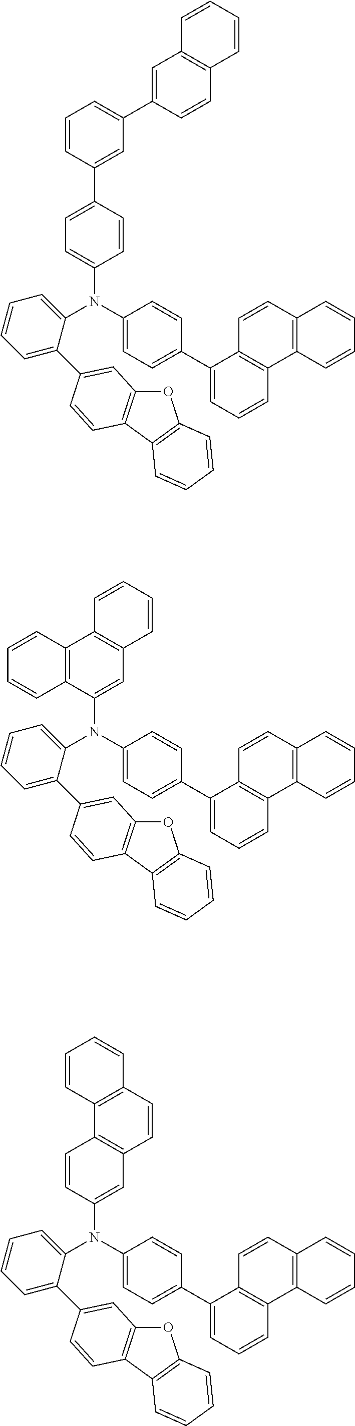 [SMILES: c1cc(-c2ccc(N(c3ccc(-c4cccc5c4ccc4ccccc45)cc3)c3ccccc3-c3ccc4c(c3)oc3ccccc34)cc2)cc(-c2ccc3ccccc3c2)c1 ; c1ccc(N(c2ccc(-c3cccc4c3ccc3ccccc34)cc2)c2cc3ccccc3c3ccccc23)c(-c2ccc3c(c2)oc2ccccc23)c1 ; c1ccc(N(c2ccc(-c3cccc4c3ccc3ccccc34)cc2)c2ccc3c(ccc4ccccc43)c2)c(-c2ccc3c(c2)oc2ccccc23)c1]

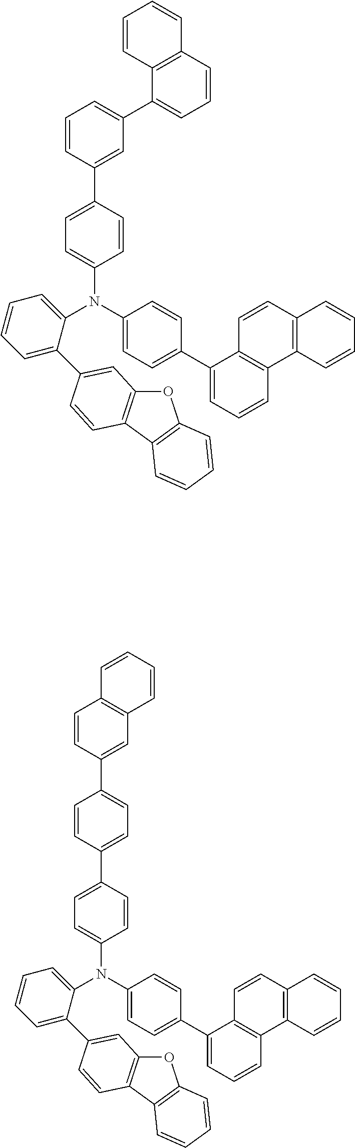 [SMILES: c1cc(-c2ccc(N(c3ccc(-c4cccc5c4ccc4ccccc45)cc3)c3ccccc3-c3ccc4c(c3)oc3ccccc34)cc2)cc(-c2cccc3ccccc23)c1 ; c1ccc(N(c2ccc(-c3ccc(-c4ccc5ccccc5c4)cc3)cc2)c2ccc(-c3cccc4c3ccc3ccccc34)cc2)c(-c2ccc3c(c2)oc2ccccc23)c1]

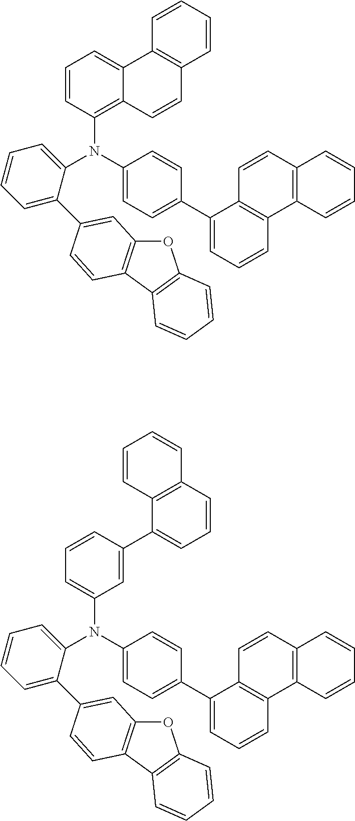 [SMILES: c1ccc(N(c2ccc(-c3cccc4c3ccc3ccccc34)cc2)c2cccc3c2ccc2ccccc23)c(-c2ccc3c(c2)oc2ccccc23)c1 ; c1cc(-c2cccc3ccccc23)cc(N(c2ccc(-c3cccc4c3ccc3ccccc34)cc2)c2ccccc2-c2ccc3c(c2)oc2ccccc23)c1]

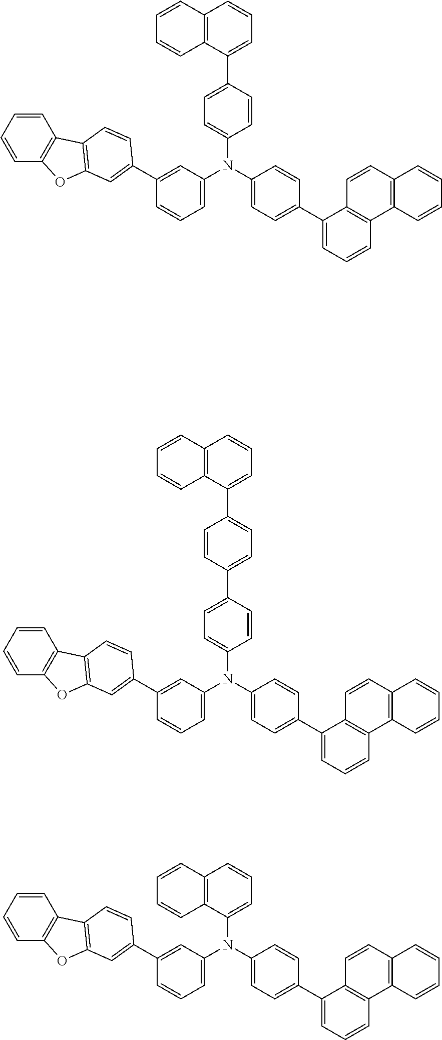 [SMILES: c1cc(-c2ccc3c(c2)oc2ccccc23)cc(N(c2ccc(-c3cccc4ccccc34)cc2)c2ccc(-c3cccc4c3ccc3ccccc34)cc2)c1 ; c1cc(-c2ccc3c(c2)oc2ccccc23)cc(N(c2ccc(-c3ccc(-c4cccc5ccccc45)cc3)cc2)c2ccc(-c3cccc4c3ccc3ccccc34)cc2)c1 ; c1cc(-c2ccc3c(c2)oc2ccccc23)cc(N(c2ccc(-c3cccc4c3ccc3ccccc34)cc2)c2cccc3ccccc23)c1]

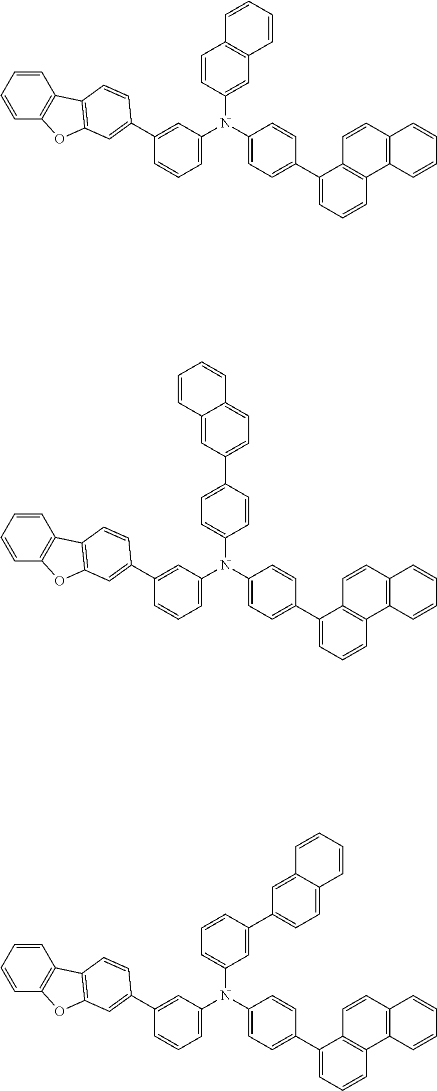 [SMILES: c1cc(-c2ccc3c(c2)oc2ccccc23)cc(N(c2ccc(-c3cccc4c3ccc3ccccc34)cc2)c2ccc3ccccc3c2)c1 ; c1cc(-c2ccc3c(c2)oc2ccccc23)cc(N(c2ccc(-c3ccc4ccccc4c3)cc2)c2ccc(-c3cccc4c3ccc3ccccc34)cc2)c1 ; c1cc(-c2ccc3ccccc3c2)cc(N(c2ccc(-c3cccc4c3ccc3ccccc34)cc2)c2cccc(-c3ccc4c(c3)oc3ccccc34)c2)c1]

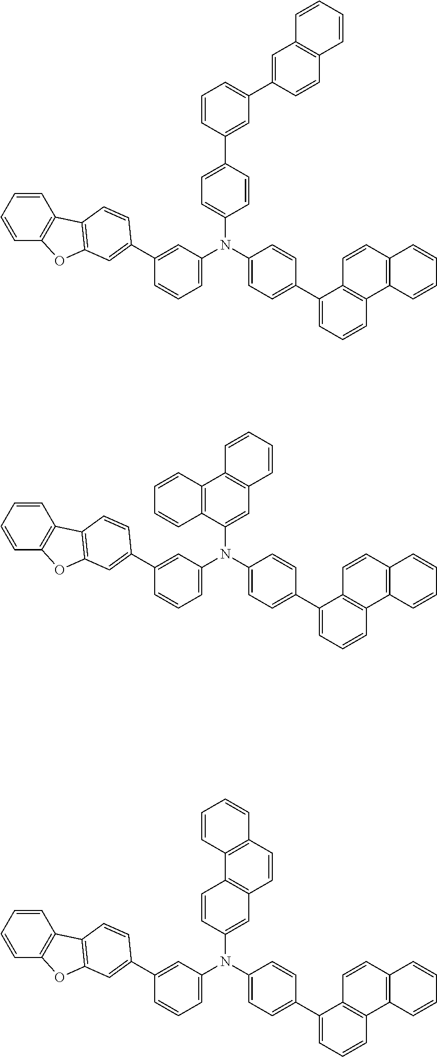 [SMILES: c1cc(-c2ccc(N(c3ccc(-c4cccc5c4ccc4ccccc45)cc3)c3cccc(-c4ccc5c(c4)oc4ccccc45)c3)cc2)cc(-c2ccc3ccccc3c2)c1 ; c1cc(-c2ccc3c(c2)oc2ccccc23)cc(N(c2ccc(-c3cccc4c3ccc3ccccc34)cc2)c2cc3ccccc3c3ccccc23)c1 ; c1cc(-c2ccc3c(c2)oc2ccccc23)cc(N(c2ccc(-c3cccc4c3ccc3ccccc34)cc2)c2ccc3c(ccc4ccccc43)c2)c1]

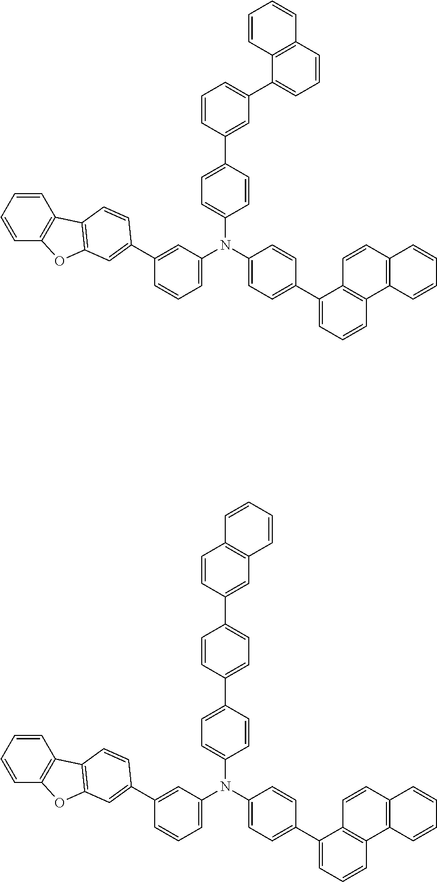 [SMILES: c1cc(-c2ccc(N(c3ccc(-c4cccc5c4ccc4ccccc45)cc3)c3cccc(-c4ccc5c(c4)oc4ccccc45)c3)cc2)cc(-c2cccc3ccccc23)c1 ; c1cc(-c2ccc3c(c2)oc2ccccc23)cc(N(c2ccc(-c3ccc(-c4ccc5ccccc5c4)cc3)cc2)c2ccc(-c3cccc4c3ccc3ccccc34)cc2)c1]

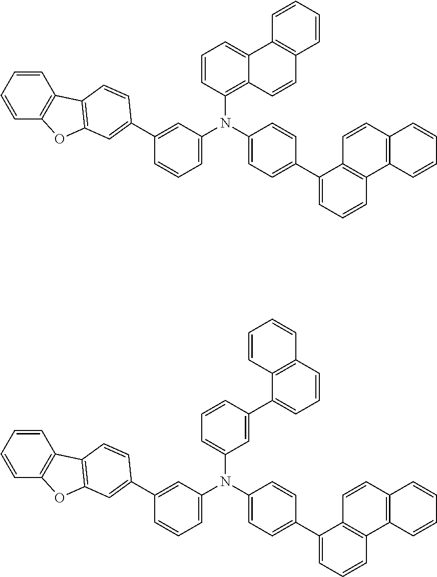 [SMILES: c1cc(-c2ccc3c(c2)oc2ccccc23)cc(N(c2ccc(-c3cccc4c3ccc3ccccc34)cc2)c2cccc3c2ccc2ccccc23)c1 ; c1cc(-c2ccc3c(c2)oc2ccccc23)cc(N(c2ccc(-c3cccc4c3ccc3ccccc34)cc2)c2cccc(-c3cccc4ccccc34)c2)c1]

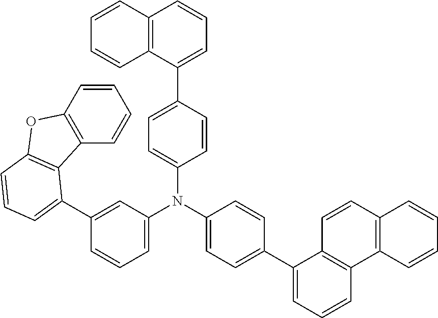 [SMILES: c1cc(-c2cccc3oc4ccccc4c23)cc(N(c2ccc(-c3cccc4ccccc34)cc2)c2ccc(-c3cccc4c3ccc3ccccc34)cc2)c1]

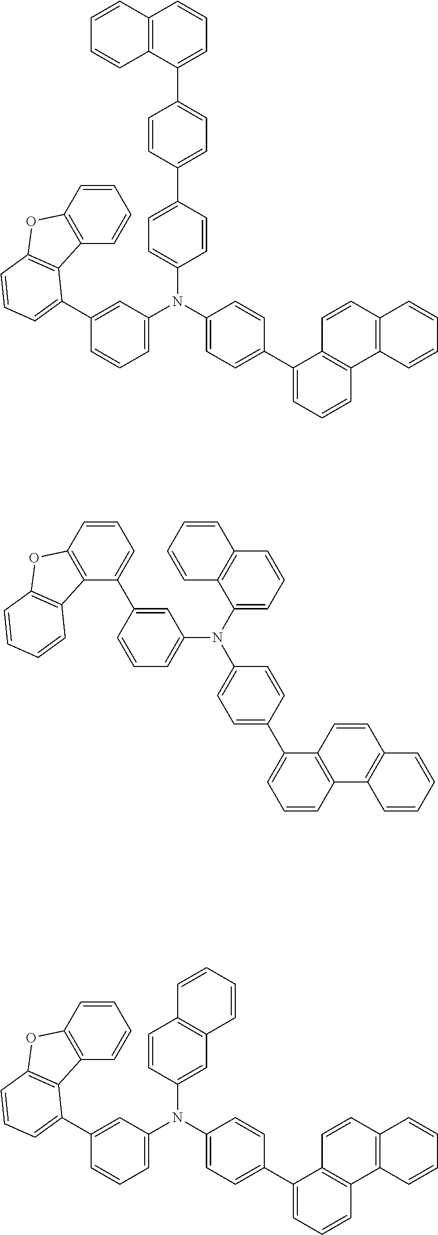 [SMILES: c1cc(-c2cccc3oc4ccccc4c23)cc(N(c2ccc(-c3ccc(-c4cccc5ccccc45)cc3)cc2)c2ccc(-c3cccc4c3ccc3ccccc34)cc2)c1 ; c1cc(-c2cccc3oc4ccccc4c23)cc(N(c2ccc(-c3cccc4c3ccc3ccccc34)cc2)c2cccc3ccccc23)c1 ; c1cc(-c2cccc3oc4ccccc4c23)cc(N(c2ccc(-c3cccc4c3ccc3ccccc34)cc2)c2ccc3ccccc3c2)c1]

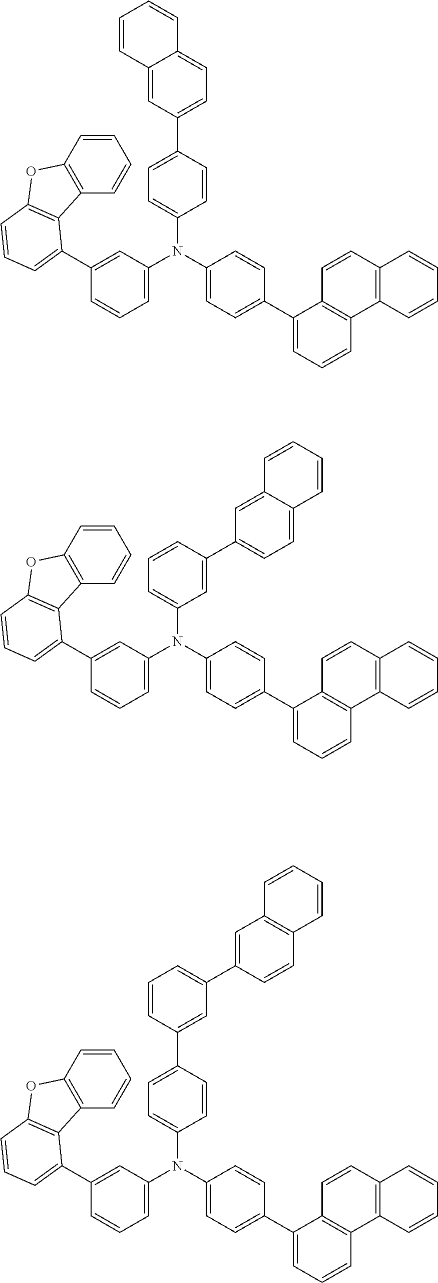 [SMILES: c1cc(-c2cccc3oc4ccccc4c23)cc(N(c2ccc(-c3ccc4ccccc4c3)cc2)c2ccc(-c3cccc4c3ccc3ccccc34)cc2)c1 ; c1cc(-c2ccc3ccccc3c2)cc(N(c2ccc(-c3cccc4c3ccc3ccccc34)cc2)c2cccc(-c3cccc4oc5ccccc5c34)c2)c1 ; c1cc(-c2ccc(N(c3ccc(-c4cccc5c4ccc4ccccc45)cc3)c3cccc(-c4cccc5oc6ccccc6c45)c3)cc2)cc(-c2ccc3ccccc3c2)c1]

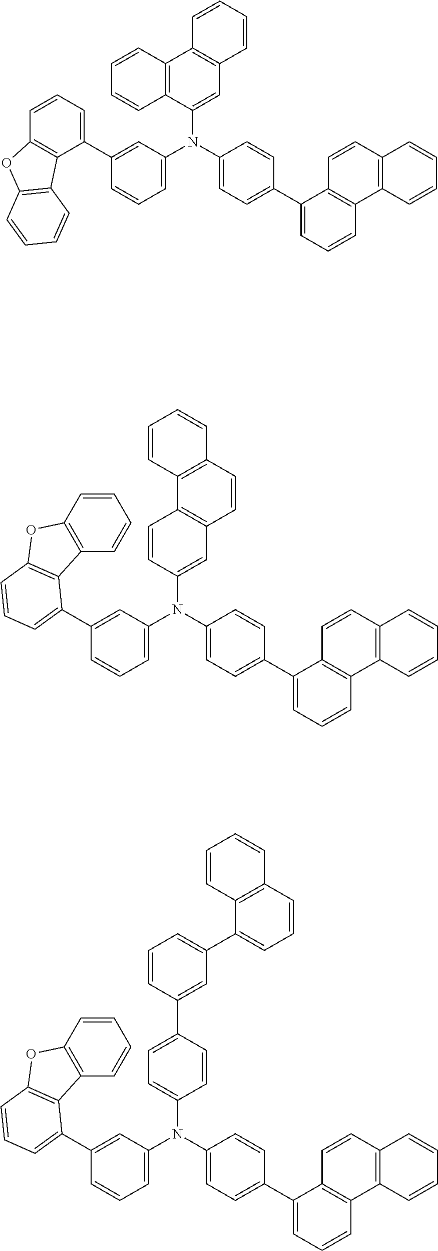 [SMILES: c1cc(-c2cccc3oc4ccccc4c23)cc(N(c2ccc(-c3cccc4c3ccc3ccccc34)cc2)c2cc3ccccc3c3ccccc23)c1 ; c1cc(-c2cccc3oc4ccccc4c23)cc(N(c2ccc(-c3cccc4c3ccc3ccccc34)cc2)c2ccc3c(ccc4ccccc43)c2)c1 ; c1cc(-c2ccc(N(c3ccc(-c4cccc5c4ccc4ccccc45)cc3)c3cccc(-c4cccc5oc6ccccc6c45)c3)cc2)cc(-c2cccc3ccccc23)c1]

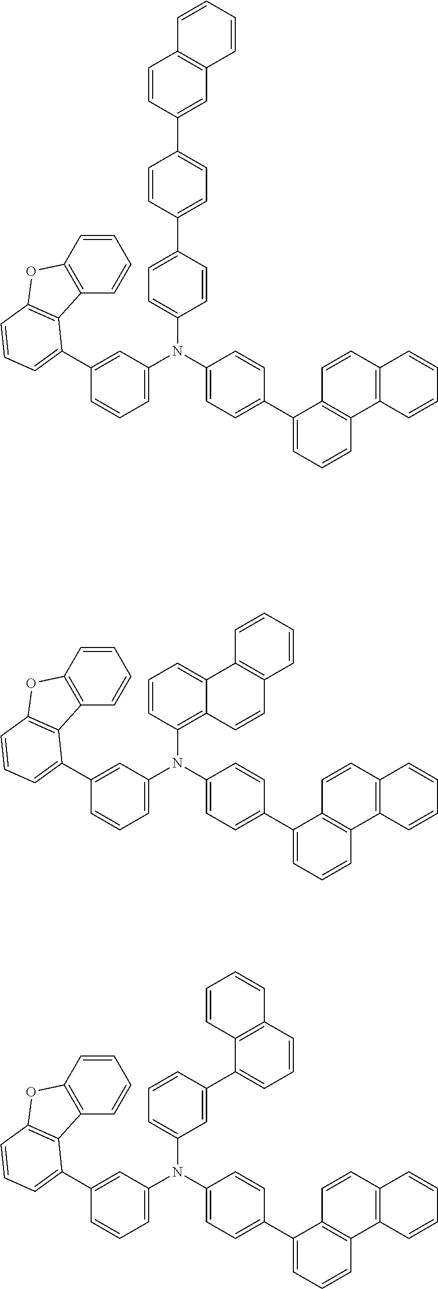 [SMILES: c1cc(-c2cccc3oc4ccccc4c23)cc(N(c2ccc(-c3ccc(-c4ccc5ccccc5c4)cc3)cc2)c2ccc(-c3cccc4c3ccc3ccccc34)cc2)c1 ; c1cc(-c2cccc3oc4ccccc4c23)cc(N(c2ccc(-c3cccc4c3ccc3ccccc34)cc2)c2cccc3c2ccc2ccccc23)c1 ; c1cc(-c2cccc3ccccc23)cc(N(c2ccc(-c3cccc4c3ccc3ccccc34)cc2)c2cccc(-c3cccc4oc5ccccc5c34)c2)c1]

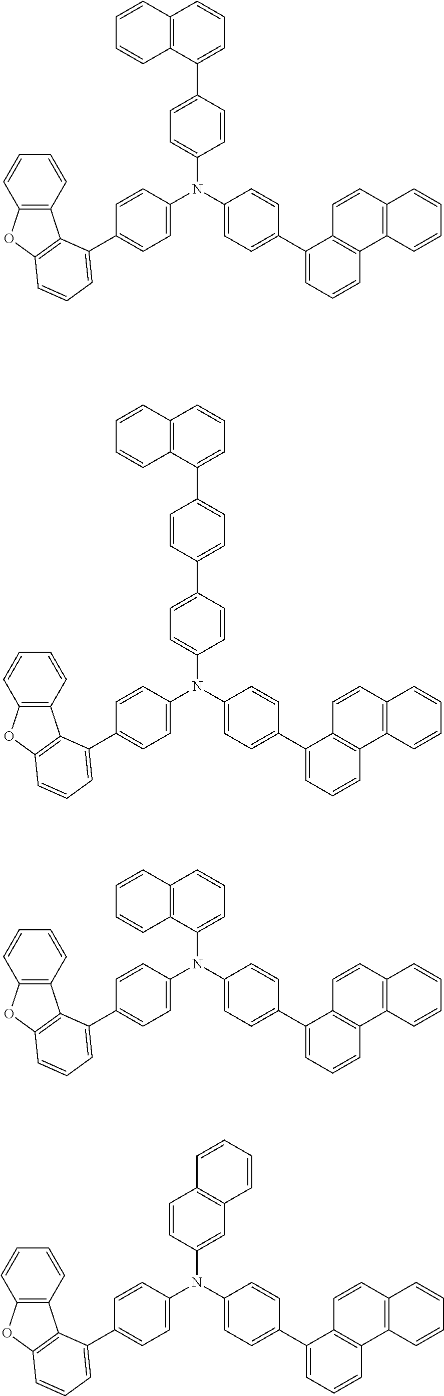 [SMILES: c1ccc2c(-c3ccc(N(c4ccc(-c5cccc6c5ccc5ccccc56)cc4)c4ccc(-c5cccc6oc7ccccc7c56)cc4)cc3)cccc2c1 ; c1ccc2c(-c3ccc(-c4ccc(N(c5ccc(-c6cccc7c6ccc6ccccc67)cc5)c5ccc(-c6cccc7oc8ccccc8c67)cc5)cc4)cc3)cccc2c1 ; c1ccc2c(N(c3ccc(-c4cccc5c4ccc4ccccc45)cc3)c3ccc(-c4cccc5oc6ccccc6c45)cc3)cccc2c1 ; c1ccc2cc(N(c3ccc(-c4cccc5c4ccc4ccccc45)cc3)c3ccc(-c4cccc5oc6ccccc6c45)cc3)ccc2c1]

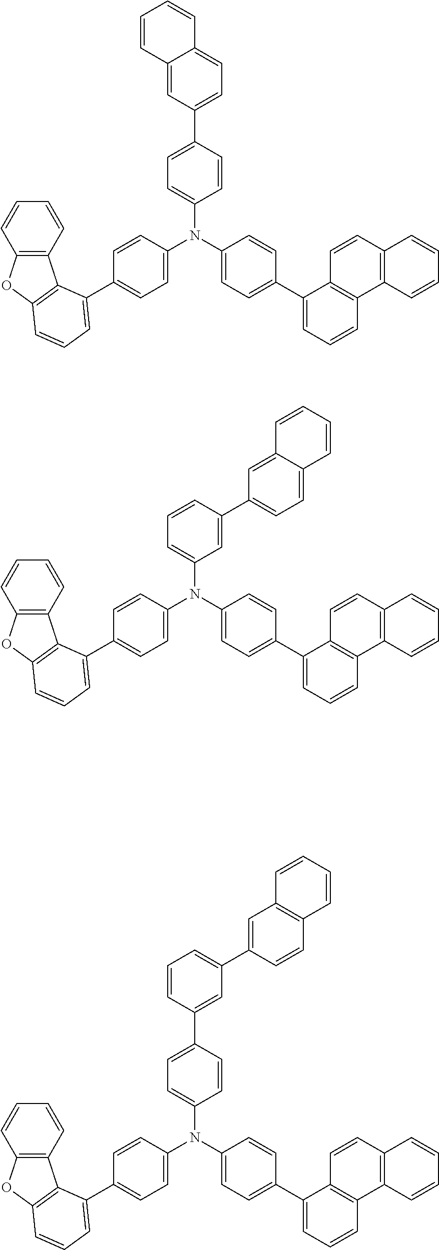 [SMILES: c1ccc2cc(-c3ccc(N(c4ccc(-c5cccc6c5ccc5ccccc56)cc4)c4ccc(-c5cccc6oc7ccccc7c56)cc4)cc3)ccc2c1 ; c1cc(-c2ccc3ccccc3c2)cc(N(c2ccc(-c3cccc4c3ccc3ccccc34)cc2)c2ccc(-c3cccc4oc5ccccc5c34)cc2)c1 ; c1cc(-c2ccc(N(c3ccc(-c4cccc5c4ccc4ccccc45)cc3)c3ccc(-c4cccc5oc6ccccc6c45)cc3)cc2)cc(-c2ccc3ccccc3c2)c1]

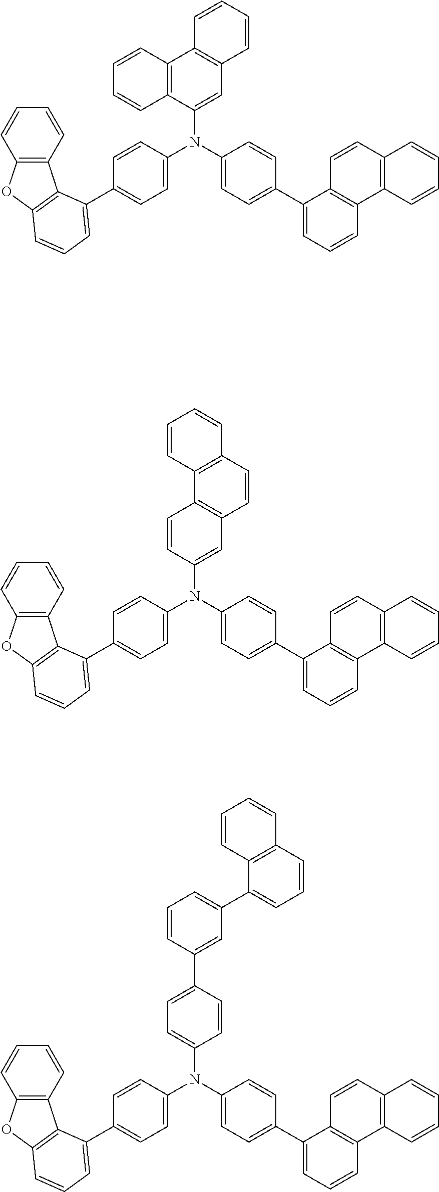 [SMILES: c1ccc2c(c1)ccc1c(-c3ccc(N(c4ccc(-c5cccc6oc7ccccc7c56)cc4)c4cc5ccccc5c5ccccc45)cc3)cccc12 ; c1ccc2c(c1)ccc1cc(N(c3ccc(-c4cccc5c4ccc4ccccc45)cc3)c3ccc(-c4cccc5oc6ccccc6c45)cc3)ccc12 ; c1cc(-c2ccc(N(c3ccc(-c4cccc5c4ccc4ccccc45)cc3)c3ccc(-c4cccc5oc6ccccc6c45)cc3)cc2)cc(-c2cccc3ccccc23)c1]

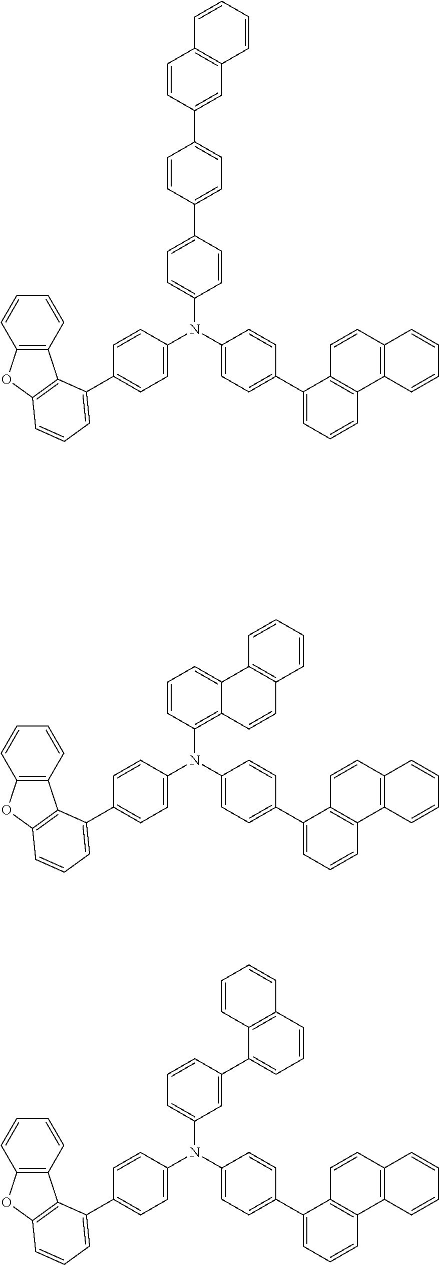 [SMILES: c1ccc2cc(-c3ccc(-c4ccc(N(c5ccc(-c6cccc7c6ccc6ccccc67)cc5)c5ccc(-c6cccc7oc8ccccc8c67)cc5)cc4)cc3)ccc2c1 ; c1ccc2c(c1)ccc1c(-c3ccc(N(c4ccc(-c5cccc6oc7ccccc7c56)cc4)c4cccc5c4ccc4ccccc45)cc3)cccc12 ; c1cc(-c2cccc3ccccc23)cc(N(c2ccc(-c3cccc4c3ccc3ccccc34)cc2)c2ccc(-c3cccc4oc5ccccc5c34)cc2)c1]

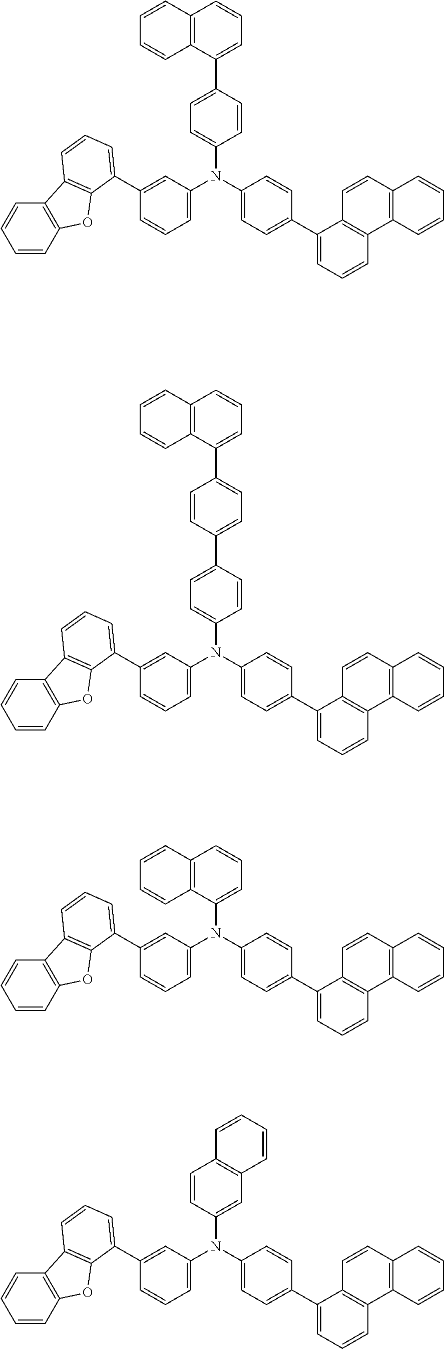 [SMILES: c1cc(-c2cccc3c2oc2ccccc23)cc(N(c2ccc(-c3cccc4ccccc34)cc2)c2ccc(-c3cccc4c3ccc3ccccc34)cc2)c1 ; c1cc(-c2cccc3c2oc2ccccc23)cc(N(c2ccc(-c3ccc(-c4cccc5ccccc45)cc3)cc2)c2ccc(-c3cccc4c3ccc3ccccc34)cc2)c1 ; c1cc(-c2cccc3c2oc2ccccc23)cc(N(c2ccc(-c3cccc4c3ccc3ccccc34)cc2)c2cccc3ccccc23)c1 ; c1cc(-c2cccc3c2oc2ccccc23)cc(N(c2ccc(-c3cccc4c3ccc3ccccc34)cc2)c2ccc3ccccc3c2)c1]

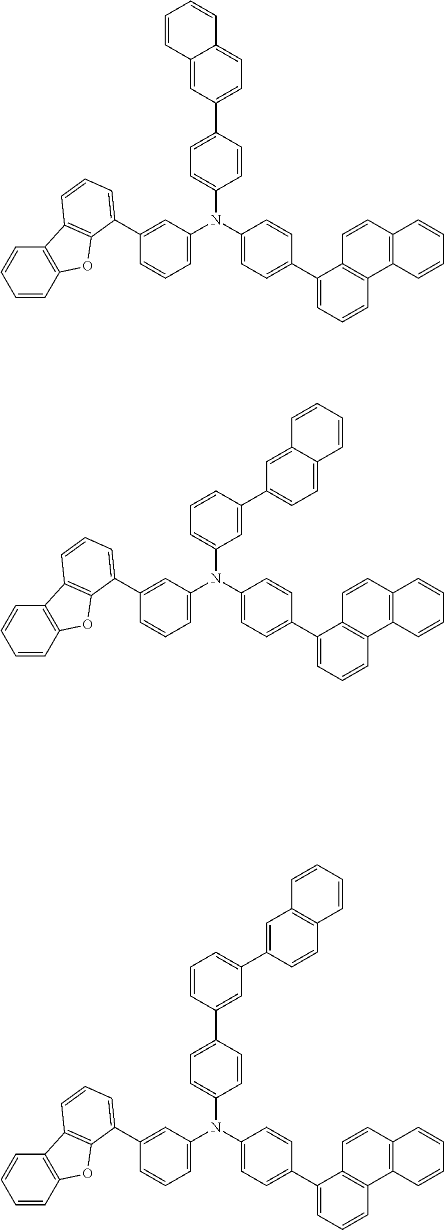 [SMILES: c1cc(-c2cccc3c2oc2ccccc23)cc(N(c2ccc(-c3ccc4ccccc4c3)cc2)c2ccc(-c3cccc4c3ccc3ccccc34)cc2)c1 ; c1cc(-c2ccc3ccccc3c2)cc(N(c2ccc(-c3cccc4c3ccc3ccccc34)cc2)c2cccc(-c3cccc4c3oc3ccccc34)c2)c1 ; c1cc(-c2ccc(N(c3ccc(-c4cccc5c4ccc4ccccc45)cc3)c3cccc(-c4cccc5c4oc4ccccc45)c3)cc2)cc(-c2ccc3ccccc3c2)c1]

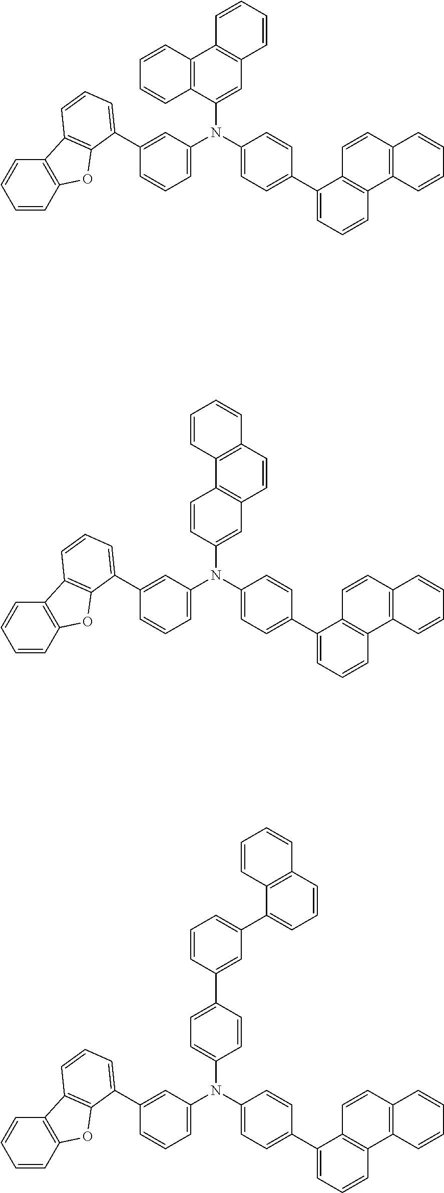 [SMILES: c1cc(-c2cccc3c2oc2ccccc23)cc(N(c2ccc(-c3cccc4c3ccc3ccccc34)cc2)c2cc3ccccc3c3ccccc23)c1 ; c1cc(-c2cccc3c2oc2ccccc23)cc(N(c2ccc(-c3cccc4c3ccc3ccccc34)cc2)c2ccc3c(ccc4ccccc43)c2)c1 ; c1cc(-c2ccc(N(c3ccc(-c4cccc5c4ccc4ccccc45)cc3)c3cccc(-c4cccc5c4oc4ccccc45)c3)cc2)cc(-c2cccc3ccccc23)c1]

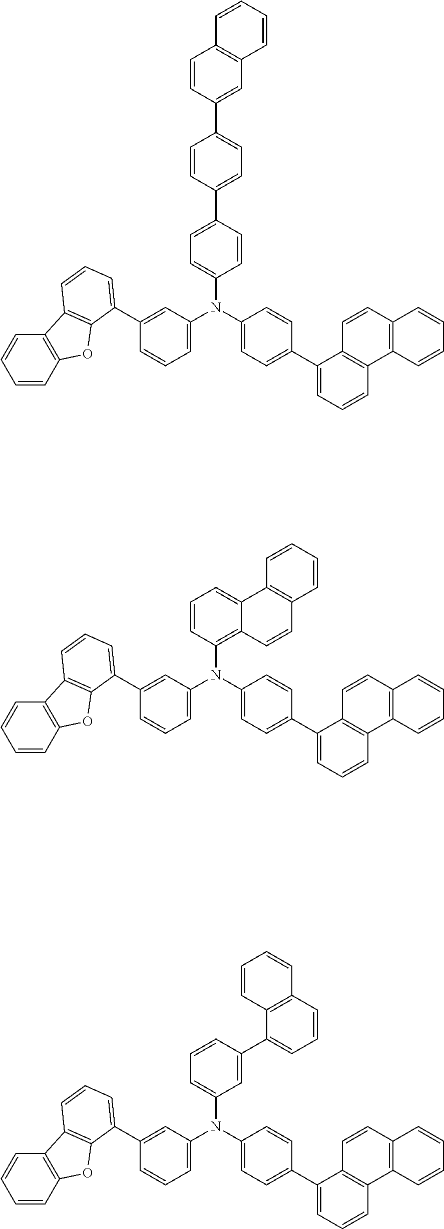 [SMILES: c1cc(-c2cccc3c2oc2ccccc23)cc(N(c2ccc(-c3ccc(-c4ccc5ccccc5c4)cc3)cc2)c2ccc(-c3cccc4c3ccc3ccccc34)cc2)c1 ; c1cc(-c2cccc3c2oc2ccccc23)cc(N(c2ccc(-c3cccc4c3ccc3ccccc34)cc2)c2cccc3c2ccc2ccccc23)c1 ; c1cc(-c2cccc3ccccc23)cc(N(c2ccc(-c3cccc4c3ccc3ccccc34)cc2)c2cccc(-c3cccc4c3oc3ccccc34)c2)c1]

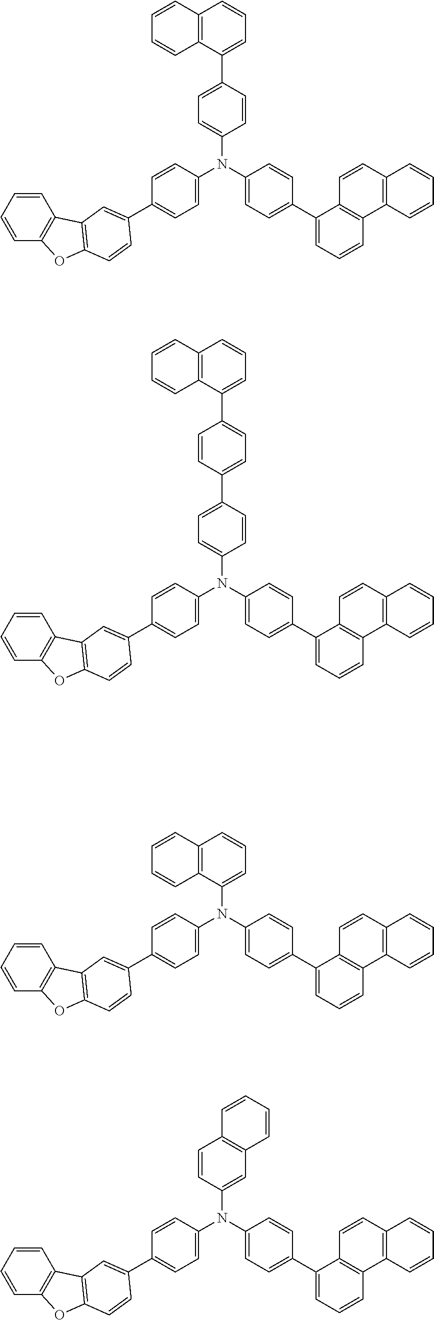 [SMILES: c1ccc2c(-c3ccc(N(c4ccc(-c5ccc6oc7ccccc7c6c5)cc4)c4ccc(-c5cccc6c5ccc5ccccc56)cc4)cc3)cccc2c1 ; c1ccc2c(-c3ccc(-c4ccc(N(c5ccc(-c6ccc7oc8ccccc8c7c6)cc5)c5ccc(-c6cccc7c6ccc6ccccc67)cc5)cc4)cc3)cccc2c1 ; c1ccc2c(N(c3ccc(-c4ccc5oc6ccccc6c5c4)cc3)c3ccc(-c4cccc5c4ccc4ccccc45)cc3)cccc2c1 ; c1ccc2cc(N(c3ccc(-c4ccc5oc6ccccc6c5c4)cc3)c3ccc(-c4cccc5c4ccc4ccccc45)cc3)ccc2c1]

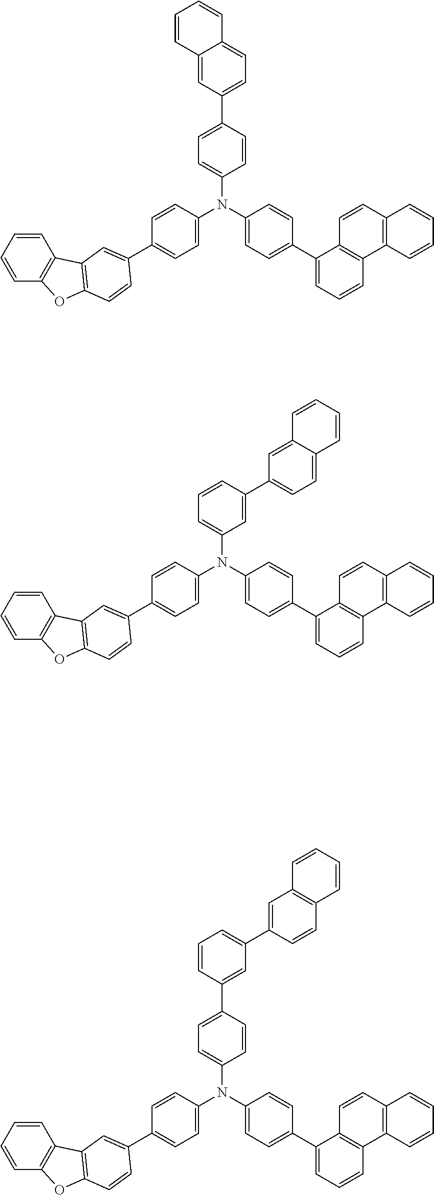 [SMILES: c1ccc2cc(-c3ccc(N(c4ccc(-c5ccc6oc7ccccc7c6c5)cc4)c4ccc(-c5cccc6c5ccc5ccccc56)cc4)cc3)ccc2c1 ; c1cc(-c2ccc3ccccc3c2)cc(N(c2ccc(-c3ccc4oc5ccccc5c4c3)cc2)c2ccc(-c3cccc4c3ccc3ccccc34)cc2)c1 ; c1cc(-c2ccc(N(c3ccc(-c4ccc5oc6ccccc6c5c4)cc3)c3ccc(-c4cccc5c4ccc4ccccc45)cc3)cc2)cc(-c2ccc3ccccc3c2)c1]

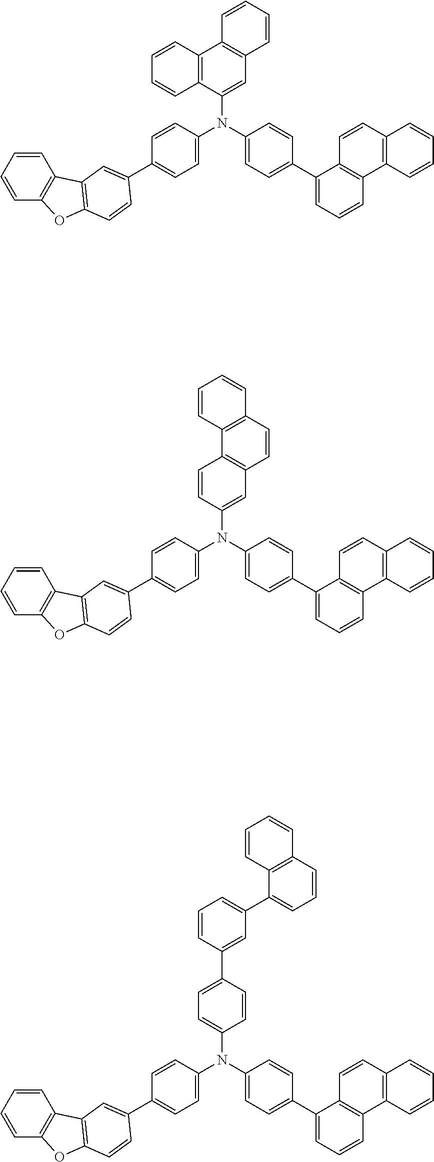 [SMILES: c1ccc2c(c1)ccc1c(-c3ccc(N(c4ccc(-c5ccc6oc7ccccc7c6c5)cc4)c4cc5ccccc5c5ccccc45)cc3)cccc12 ; c1ccc2c(c1)ccc1cc(N(c3ccc(-c4ccc5oc6ccccc6c5c4)cc3)c3ccc(-c4cccc5c4ccc4ccccc45)cc3)ccc12 ; c1cc(-c2ccc(N(c3ccc(-c4ccc5oc6ccccc6c5c4)cc3)c3ccc(-c4cccc5c4ccc4ccccc45)cc3)cc2)cc(-c2cccc3ccccc23)c1]

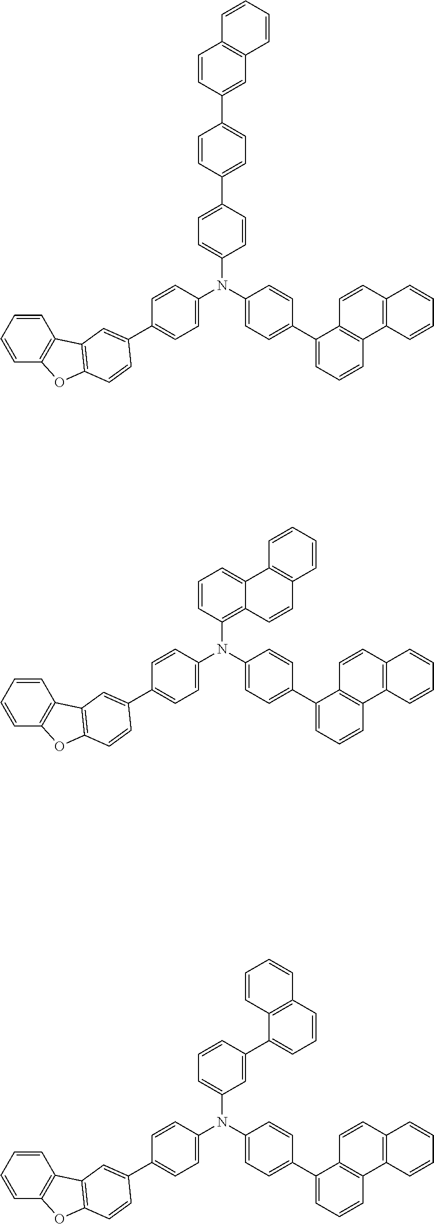 [SMILES: c1ccc2cc(-c3ccc(-c4ccc(N(c5ccc(-c6ccc7oc8ccccc8c7c6)cc5)c5ccc(-c6cccc7c6ccc6ccccc67)cc5)cc4)cc3)ccc2c1 ; c1ccc2c(c1)ccc1c(-c3ccc(N(c4ccc(-c5ccc6oc7ccccc7c6c5)cc4)c4cccc5c4ccc4ccccc45)cc3)cccc12 ; c1cc(-c2cccc3ccccc23)cc(N(c2ccc(-c3ccc4oc5ccccc5c4c3)cc2)c2ccc(-c3cccc4c3ccc3ccccc34)cc2)c1]

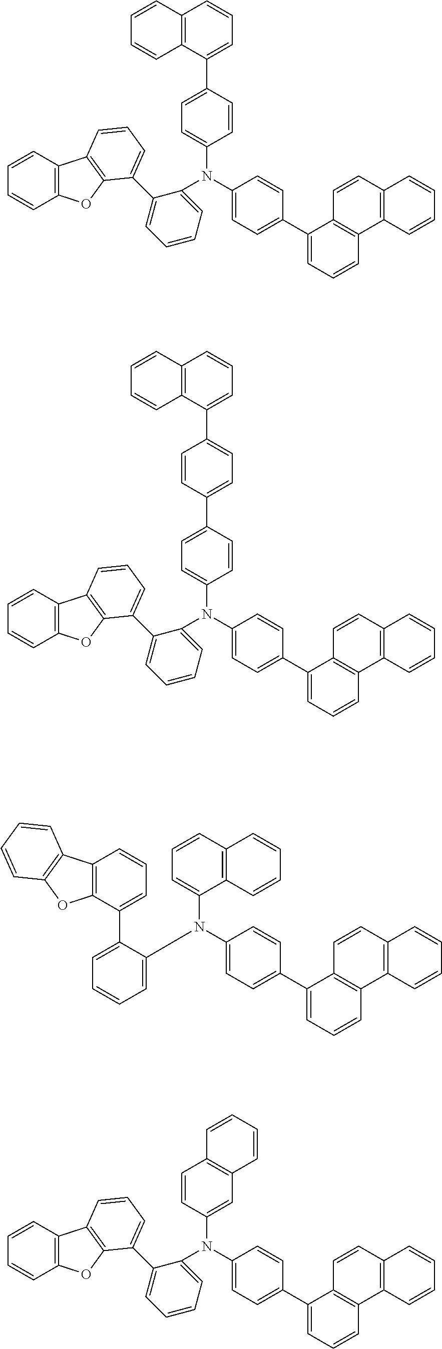 [SMILES: c1ccc(N(c2ccc(-c3cccc4ccccc34)cc2)c2ccc(-c3cccc4c3ccc3ccccc34)cc2)c(-c2cccc3c2oc2ccccc23)c1 ; c1ccc(N(c2ccc(-c3ccc(-c4cccc5ccccc45)cc3)cc2)c2ccc(-c3cccc4c3ccc3ccccc34)cc2)c(-c2cccc3c2oc2ccccc23)c1 ; c1ccc(N(c2ccc(-c3cccc4c3ccc3ccccc34)cc2)c2cccc3ccccc23)c(-c2cccc3c2oc2ccccc23)c1 ; c1ccc(N(c2ccc(-c3cccc4c3ccc3ccccc34)cc2)c2ccc3ccccc3c2)c(-c2cccc3c2oc2ccccc23)c1]

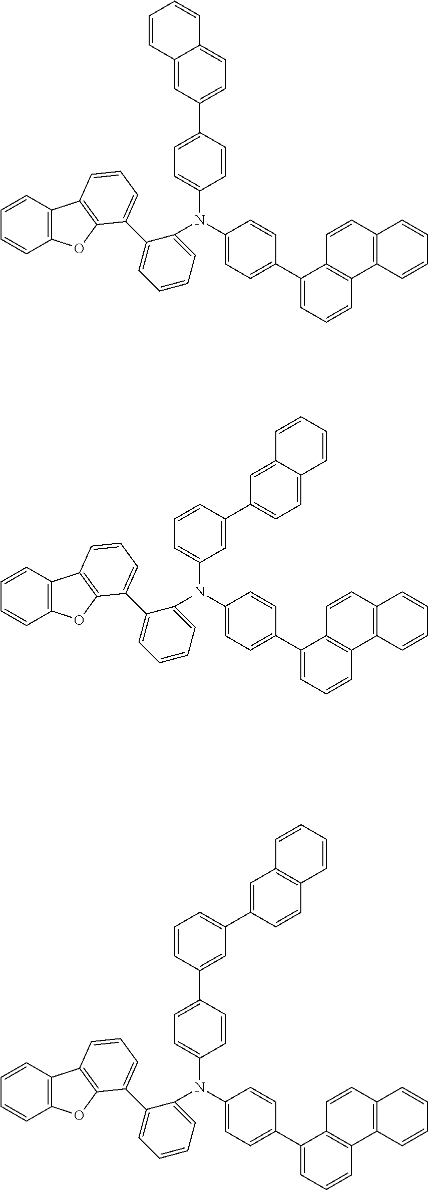 [SMILES: c1ccc(N(c2ccc(-c3ccc4ccccc4c3)cc2)c2ccc(-c3cccc4c3ccc3ccccc34)cc2)c(-c2cccc3c2oc2ccccc23)c1 ; c1cc(-c2ccc3ccccc3c2)cc(N(c2ccc(-c3cccc4c3ccc3ccccc34)cc2)c2ccccc2-c2cccc3c2oc2ccccc23)c1 ; c1cc(-c2ccc(N(c3ccc(-c4cccc5c4ccc4ccccc45)cc3)c3ccccc3-c3cccc4c3oc3ccccc34)cc2)cc(-c2ccc3ccccc3c2)c1]

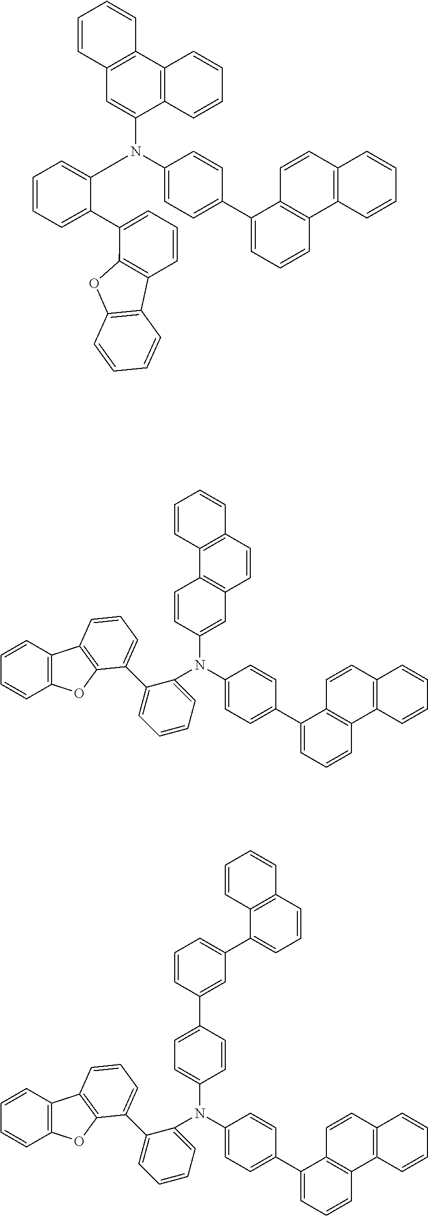 [SMILES: c1ccc(N(c2ccc(-c3cccc4c3ccc3ccccc34)cc2)c2cc3ccccc3c3ccccc23)c(-c2cccc3c2oc2ccccc23)c1 ; c1ccc(N(c2ccc(-c3cccc4c3ccc3ccccc34)cc2)c2ccc3c(ccc4ccccc43)c2)c(-c2cccc3c2oc2ccccc23)c1 ; c1cc(-c2ccc(N(c3ccc(-c4cccc5c4ccc4ccccc45)cc3)c3ccccc3-c3cccc4c3oc3ccccc34)cc2)cc(-c2cccc3ccccc23)c1]

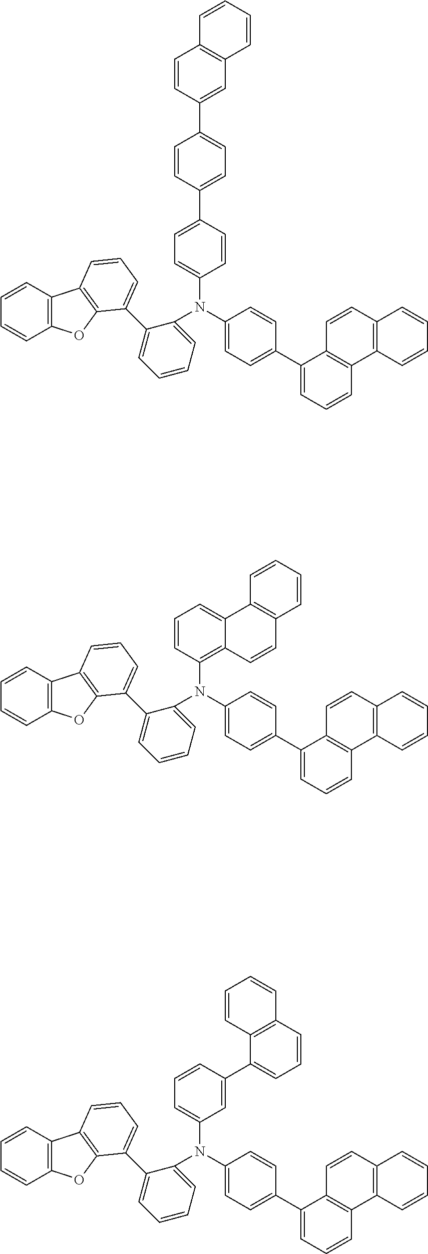 [SMILES: c1ccc(N(c2ccc(-c3ccc(-c4ccc5ccccc5c4)cc3)cc2)c2ccc(-c3cccc4c3ccc3ccccc34)cc2)c(-c2cccc3c2oc2ccccc23)c1 ; c1ccc(N(c2ccc(-c3cccc4c3ccc3ccccc34)cc2)c2cccc3c2ccc2ccccc23)c(-c2cccc3c2oc2ccccc23)c1 ; c1cc(-c2cccc3ccccc23)cc(N(c2ccc(-c3cccc4c3ccc3ccccc34)cc2)c2ccccc2-c2cccc3c2oc2ccccc23)c1]

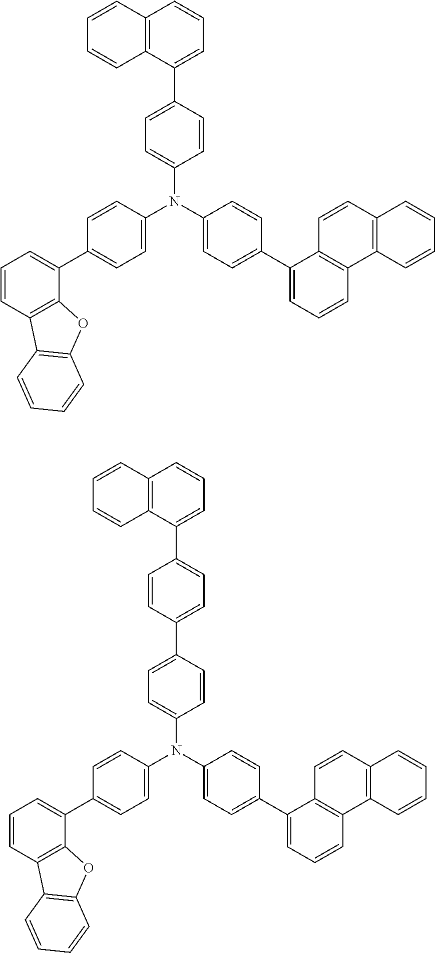 [SMILES: c1ccc2c(-c3ccc(N(c4ccc(-c5cccc6c5ccc5ccccc56)cc4)c4ccc(-c5cccc6c5oc5ccccc56)cc4)cc3)cccc2c1 ; c1ccc2c(-c3ccc(-c4ccc(N(c5ccc(-c6cccc7c6ccc6ccccc67)cc5)c5ccc(-c6cccc7c6oc6ccccc67)cc5)cc4)cc3)cccc2c1]

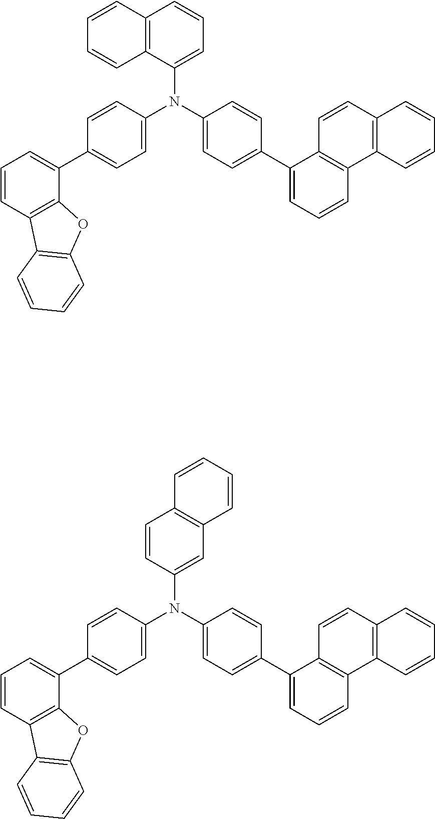 [SMILES: c1ccc2c(N(c3ccc(-c4cccc5c4ccc4ccccc45)cc3)c3ccc(-c4cccc5c4oc4ccccc45)cc3)cccc2c1 ; c1ccc2cc(N(c3ccc(-c4cccc5c4ccc4ccccc45)cc3)c3ccc(-c4cccc5c4oc4ccccc45)cc3)ccc2c1]

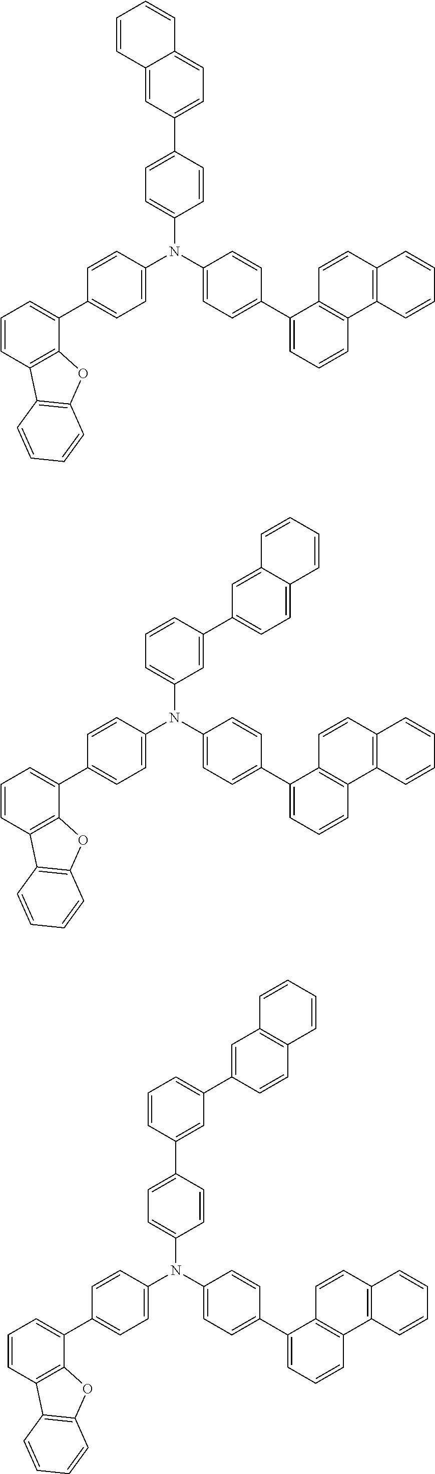 [SMILES: c1ccc2cc(-c3ccc(N(c4ccc(-c5cccc6c5ccc5ccccc56)cc4)c4ccc(-c5cccc6c5oc5ccccc56)cc4)cc3)ccc2c1 ; c1cc(-c2ccc3ccccc3c2)cc(N(c2ccc(-c3cccc4c3ccc3ccccc34)cc2)c2ccc(-c3cccc4c3oc3ccccc34)cc2)c1 ; c1cc(-c2ccc(N(c3ccc(-c4cccc5c4ccc4ccccc45)cc3)c3ccc(-c4cccc5c4oc4ccccc45)cc3)cc2)cc(-c2ccc3ccccc3c2)c1]

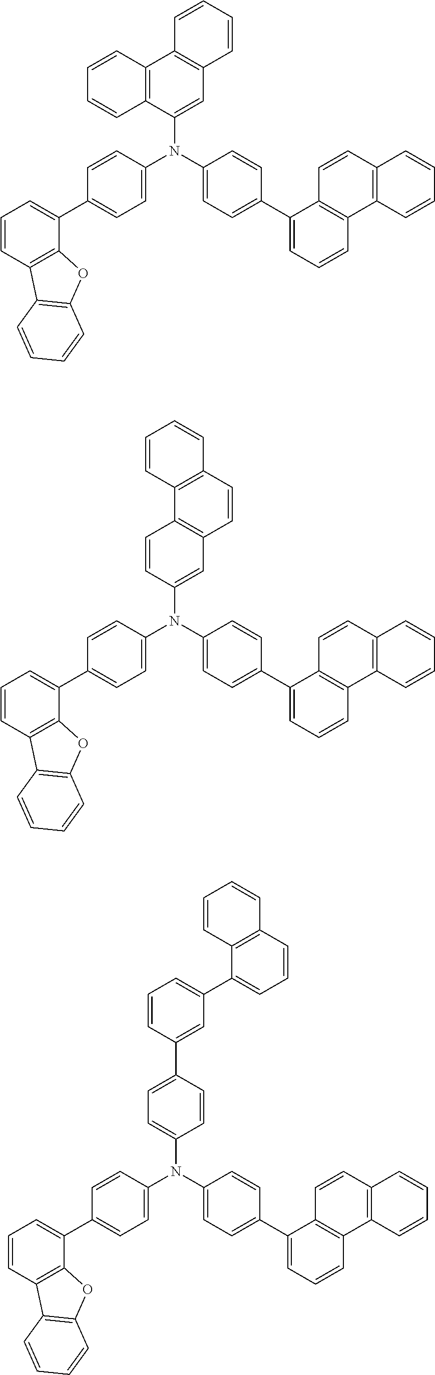 [SMILES: c1ccc2c(c1)ccc1c(-c3ccc(N(c4ccc(-c5cccc6c5oc5ccccc56)cc4)c4cc5ccccc5c5ccccc45)cc3)cccc12 ; c1ccc2c(c1)ccc1cc(N(c3ccc(-c4cccc5c4ccc4ccccc45)cc3)c3ccc(-c4cccc5c4oc4ccccc45)cc3)ccc12 ; c1cc(-c2ccc(N(c3ccc(-c4cccc5c4ccc4ccccc45)cc3)c3ccc(-c4cccc5c4oc4ccccc45)cc3)cc2)cc(-c2cccc3ccccc23)c1]

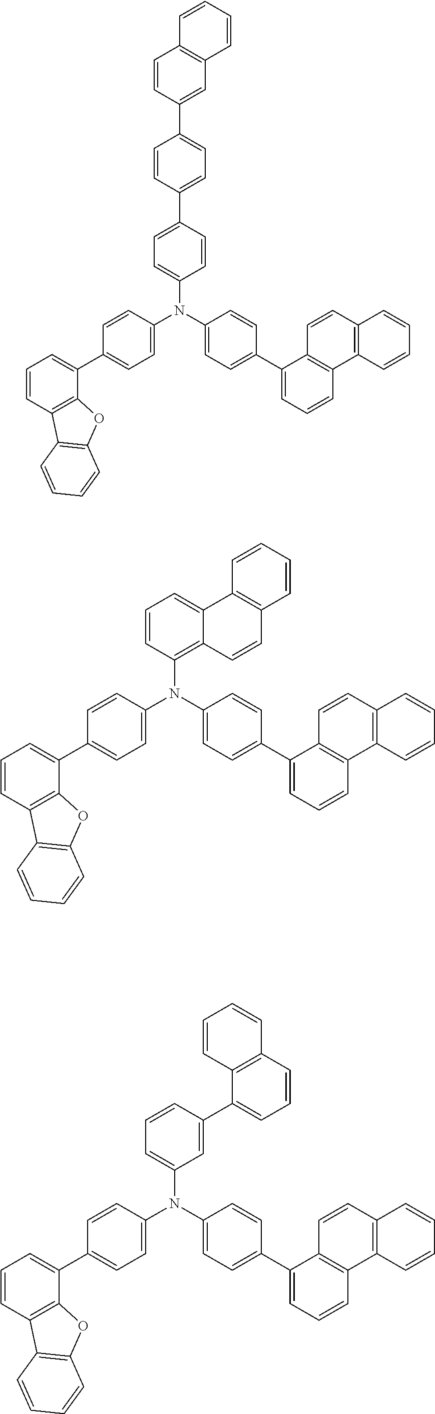 [SMILES: c1ccc2cc(-c3ccc(-c4ccc(N(c5ccc(-c6cccc7c6ccc6ccccc67)cc5)c5ccc(-c6cccc7c6oc6ccccc67)cc5)cc4)cc3)ccc2c1 ; c1ccc2c(c1)ccc1c(-c3ccc(N(c4ccc(-c5cccc6c5oc5ccccc56)cc4)c4cccc5c4ccc4ccccc45)cc3)cccc12 ; c1cc(-c2cccc3ccccc23)cc(N(c2ccc(-c3cccc4c3ccc3ccccc34)cc2)c2ccc(-c3cccc4c3oc3ccccc34)cc2)c1]

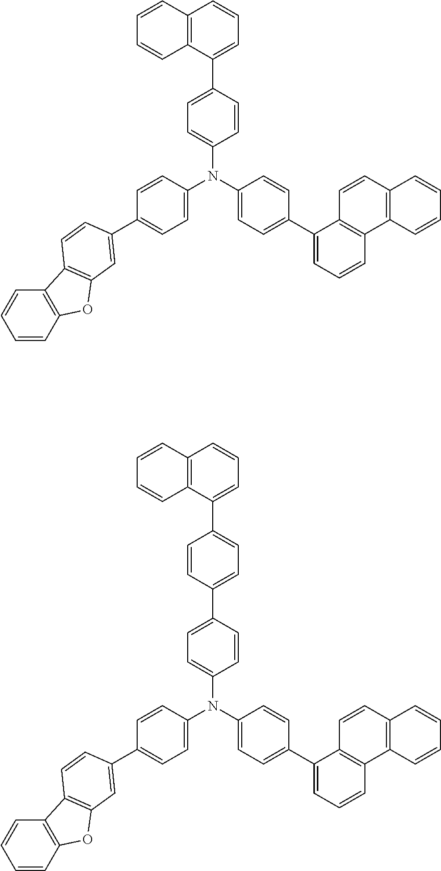 [SMILES: c1ccc2c(-c3ccc(N(c4ccc(-c5ccc6c(c5)oc5ccccc56)cc4)c4ccc(-c5cccc6c5ccc5ccccc56)cc4)cc3)cccc2c1 ; c1ccc2c(-c3ccc(-c4ccc(N(c5ccc(-c6ccc7c(c6)oc6ccccc67)cc5)c5ccc(-c6cccc7c6ccc6ccccc67)cc5)cc4)cc3)cccc2c1]

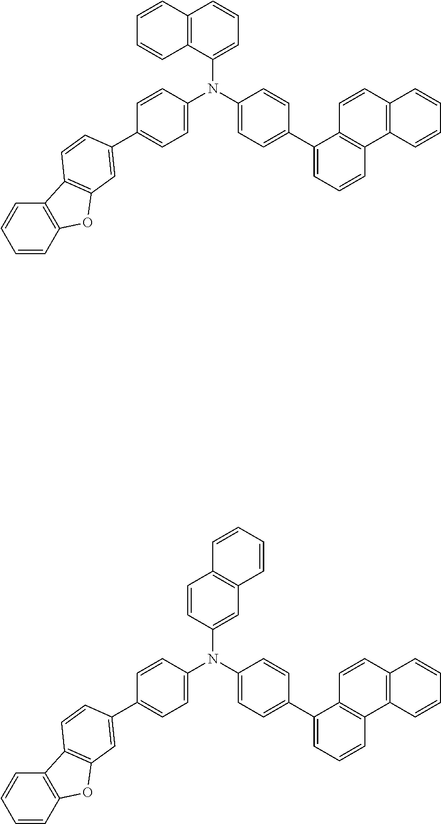 [SMILES: c1ccc2c(N(c3ccc(-c4ccc5c(c4)oc4ccccc45)cc3)c3ccc(-c4cccc5c4ccc4ccccc45)cc3)cccc2c1 ; c1ccc2cc(N(c3ccc(-c4ccc5c(c4)oc4ccccc45)cc3)c3ccc(-c4cccc5c4ccc4ccccc45)cc3)ccc2c1]

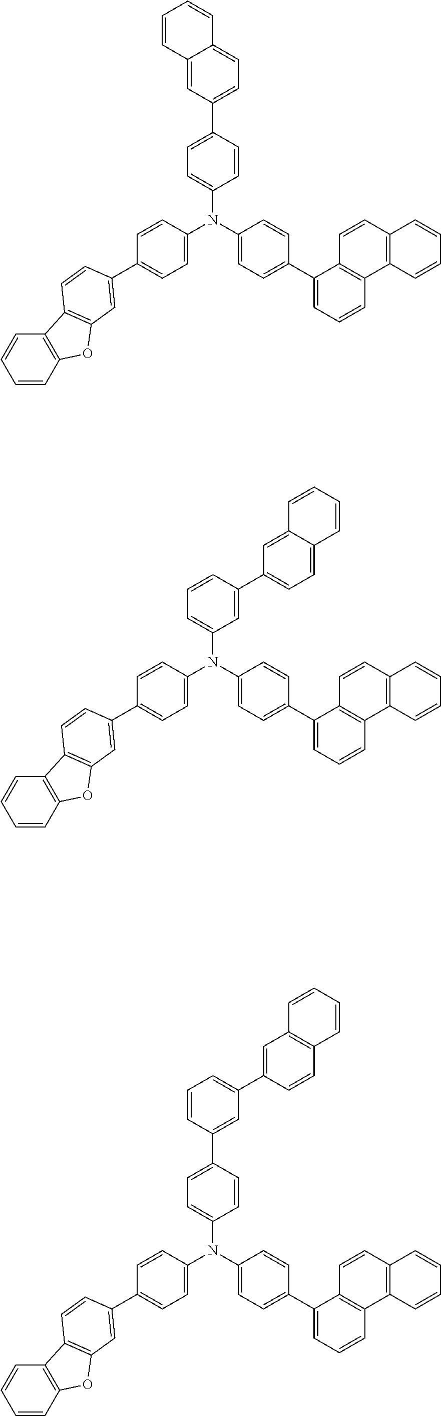 [SMILES: c1ccc2cc(-c3ccc(N(c4ccc(-c5ccc6c(c5)oc5ccccc56)cc4)c4ccc(-c5cccc6c5ccc5ccccc56)cc4)cc3)ccc2c1 ; c1cc(-c2ccc3ccccc3c2)cc(N(c2ccc(-c3ccc4c(c3)oc3ccccc34)cc2)c2ccc(-c3cccc4c3ccc3ccccc34)cc2)c1 ; c1cc(-c2ccc(N(c3ccc(-c4ccc5c(c4)oc4ccccc45)cc3)c3ccc(-c4cccc5c4ccc4ccccc45)cc3)cc2)cc(-c2ccc3ccccc3c2)c1]

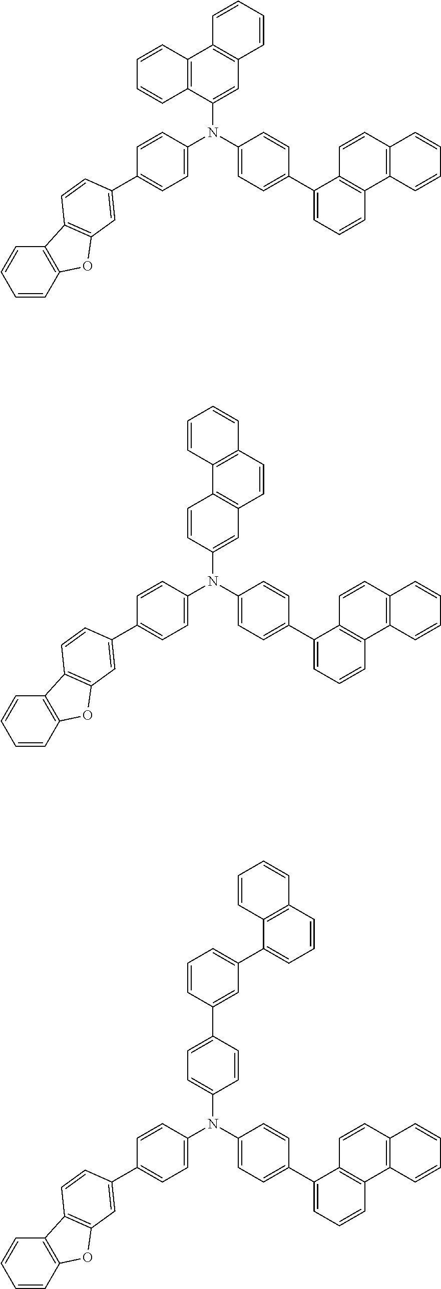 [SMILES: c1ccc2c(c1)ccc1c(-c3ccc(N(c4ccc(-c5ccc6c(c5)oc5ccccc56)cc4)c4cc5ccccc5c5ccccc45)cc3)cccc12 ; c1ccc2c(c1)ccc1cc(N(c3ccc(-c4ccc5c(c4)oc4ccccc45)cc3)c3ccc(-c4cccc5c4ccc4ccccc45)cc3)ccc12 ; c1cc(-c2ccc(N(c3ccc(-c4ccc5c(c4)oc4ccccc45)cc3)c3ccc(-c4cccc5c4ccc4ccccc45)cc3)cc2)cc(-c2cccc3ccccc23)c1]

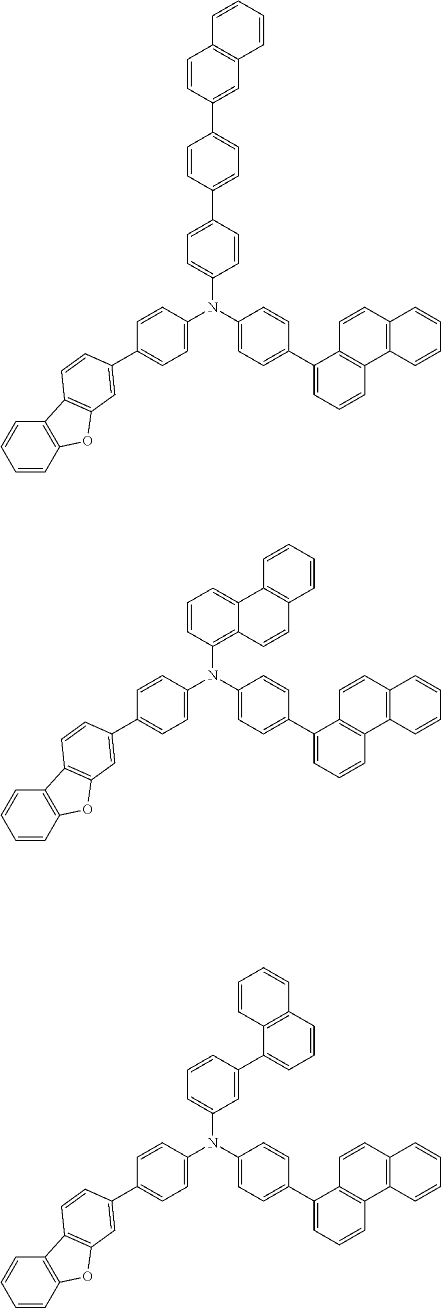 [SMILES: c1ccc2cc(-c3ccc(-c4ccc(N(c5ccc(-c6ccc7c(c6)oc6ccccc67)cc5)c5ccc(-c6cccc7c6ccc6ccccc67)cc5)cc4)cc3)ccc2c1 ; c1ccc2c(c1)ccc1c(-c3ccc(N(c4ccc(-c5ccc6c(c5)oc5ccccc56)cc4)c4cccc5c4ccc4ccccc45)cc3)cccc12 ; c1cc(-c2cccc3ccccc23)cc(N(c2ccc(-c3ccc4c(c3)oc3ccccc34)cc2)c2ccc(-c3cccc4c3ccc3ccccc34)cc2)c1]

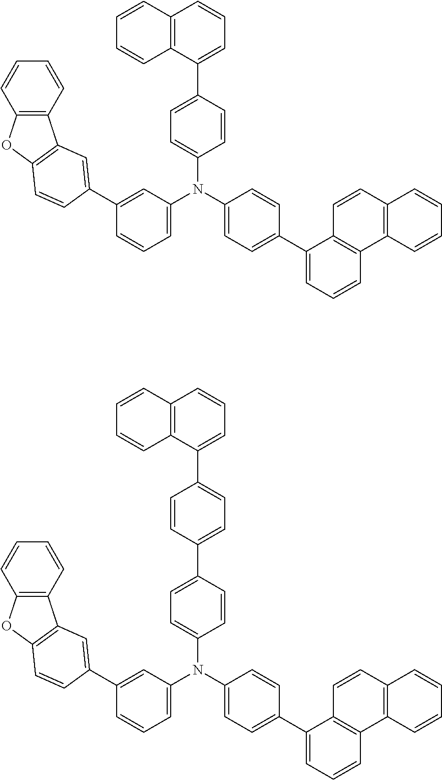

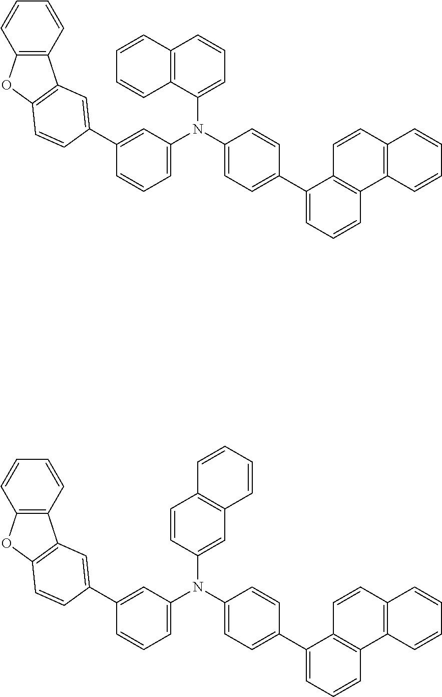

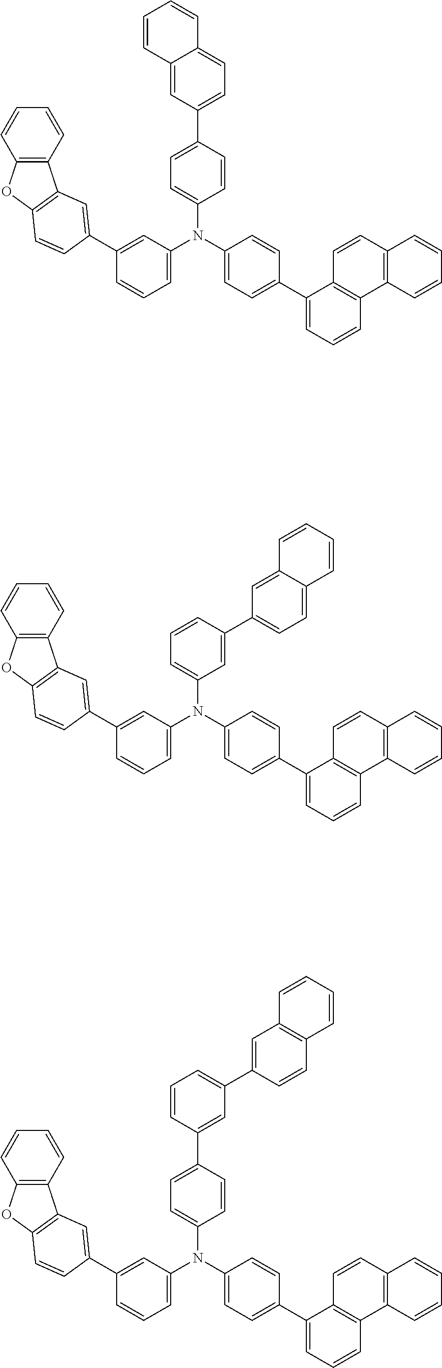

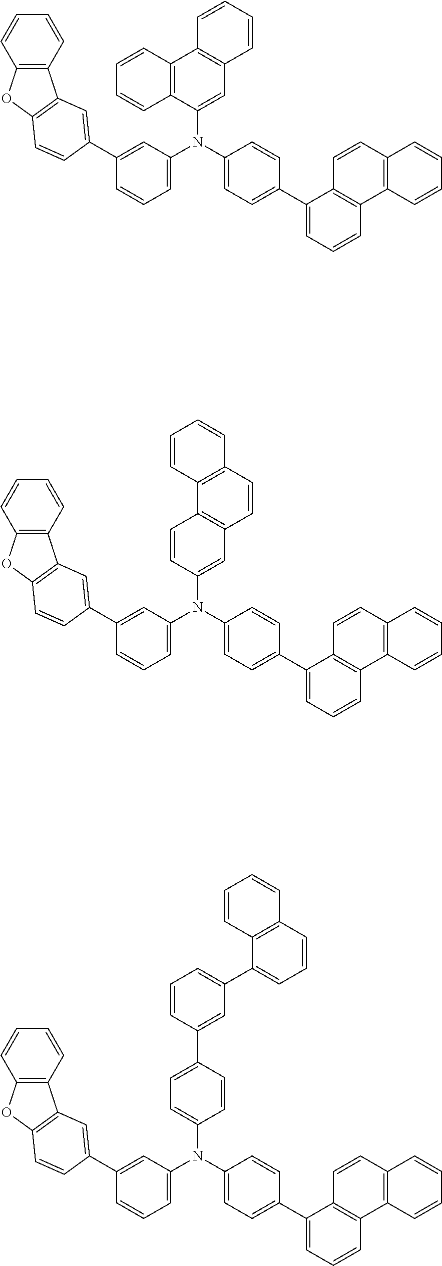

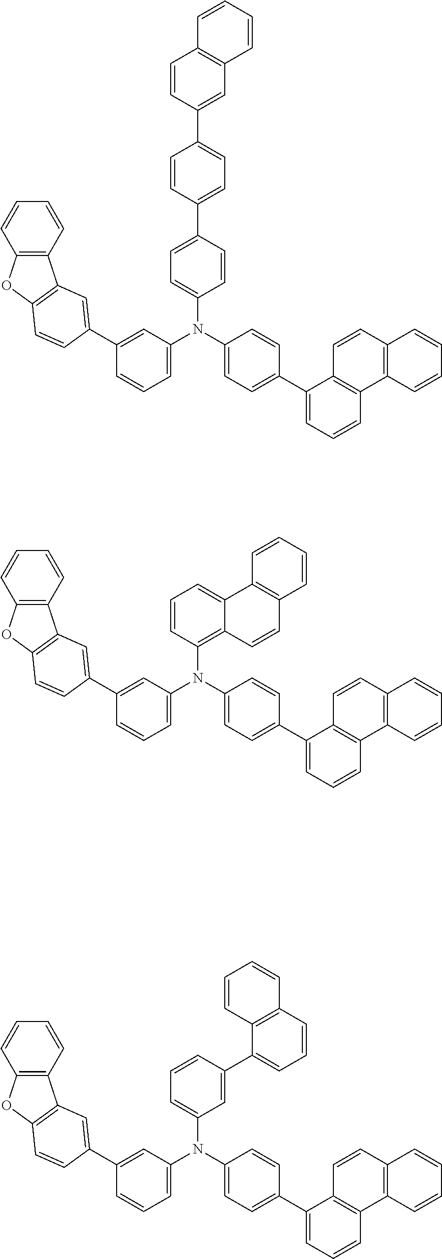

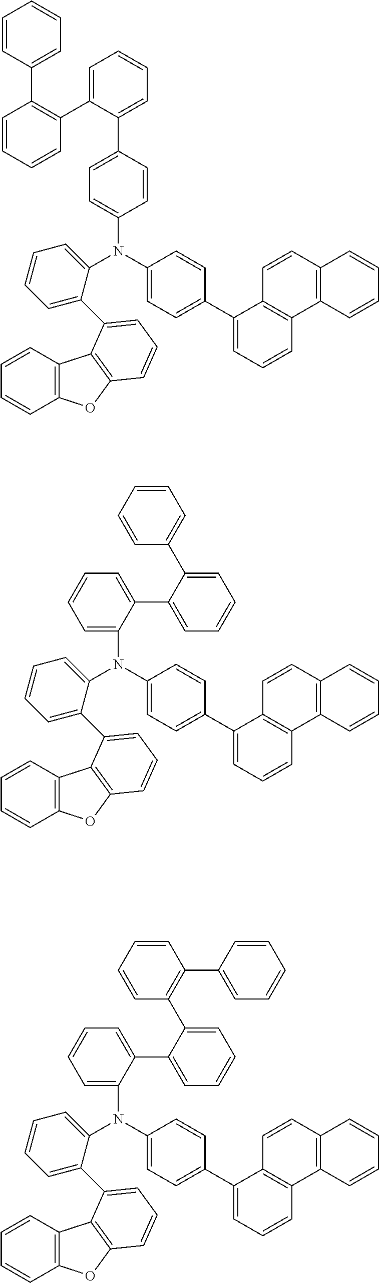 [SMILES: c1ccc(-c2ccccc2-c2ccccc2-c2ccc(N(c3ccc(-c4cccc5c4ccc4ccccc45)cc3)c3ccccc3-c3cccc4oc5ccccc5c34)cc2)cc1 ; c1ccc(-c2ccccc2-c2ccccc2N(c2ccc(-c3cccc4c3ccc3ccccc34)cc2)c2ccccc2-c2cccc3oc4ccccc4c23)cc1 ; c1ccc(-c2ccccc2-c2ccccc2-c2ccccc2N(c2ccc(-c3cccc4c3ccc3ccccc34)cc2)c2ccccc2-c2cccc3oc4ccccc4c23)cc1]

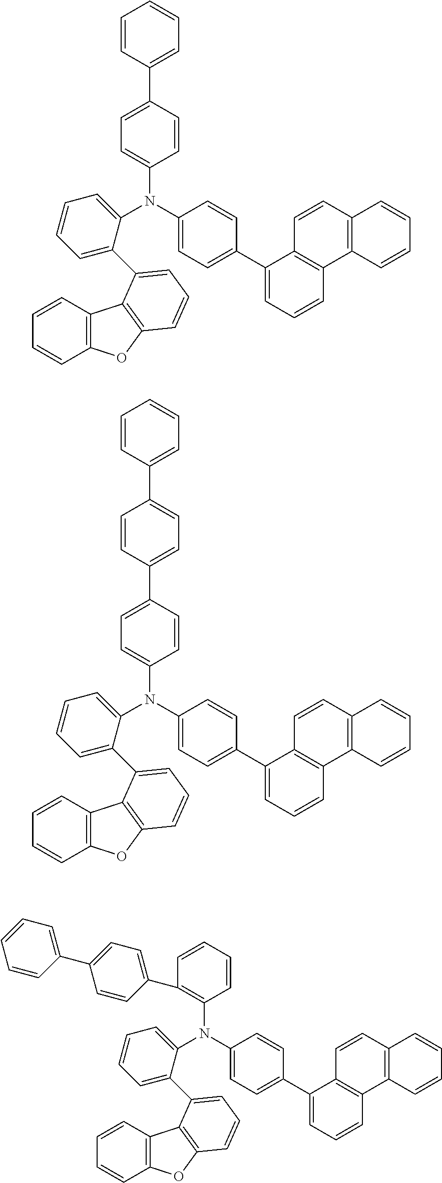 [SMILES: c1ccc(-c2ccc(N(c3ccc(-c4cccc5c4ccc4ccccc45)cc3)c3ccccc3-c3cccc4oc5ccccc5c34)cc2)cc1 ; c1ccc(-c2ccc(-c3ccc(N(c4ccc(-c5cccc6c5ccc5ccccc56)cc4)c4ccccc4-c4cccc5oc6ccccc6c45)cc3)cc2)cc1 ; c1ccc(-c2ccc(-c3ccccc3N(c3ccc(-c4cccc5c4ccc4ccccc45)cc3)c3ccccc3-c3cccc4oc5ccccc5c34)cc2)cc1]

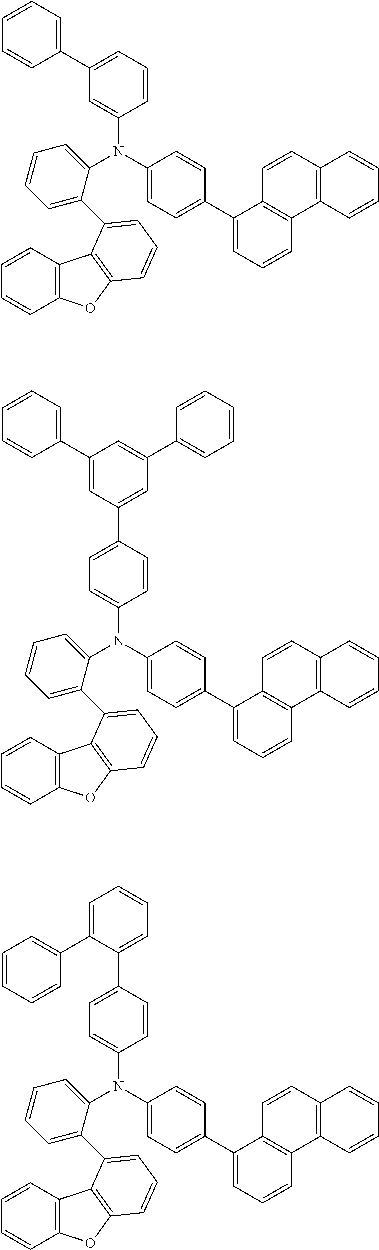 [SMILES: c1ccc(-c2cccc(N(c3ccc(-c4cccc5c4ccc4ccccc45)cc3)c3ccccc3-c3cccc4oc5ccccc5c34)c2)cc1 ; c1ccc(-c2cc(-c3ccccc3)cc(-c3ccc(N(c4ccc(-c5cccc6c5ccc5ccccc56)cc4)c4ccccc4-c4cccc5oc6ccccc6c45)cc3)c2)cc1 ; c1ccc(-c2ccccc2-c2ccc(N(c3ccc(-c4cccc5c4ccc4ccccc45)cc3)c3ccccc3-c3cccc4oc5ccccc5c34)cc2)cc1]

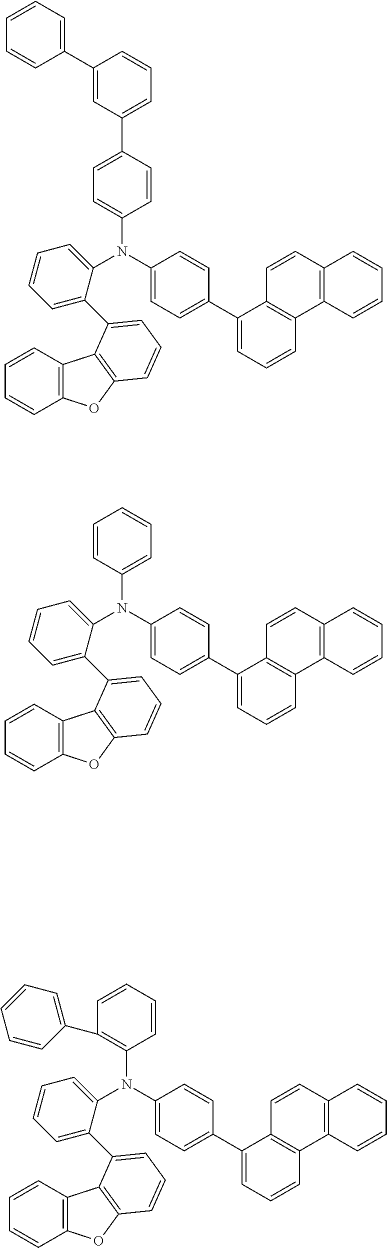 [SMILES: c1ccc(-c2cccc(-c3ccc(N(c4ccc(-c5cccc6c5ccc5ccccc56)cc4)c4ccccc4-c4cccc5oc6ccccc6c45)cc3)c2)cc1 ; c1ccc(N(c2ccc(-c3cccc4c3ccc3ccccc34)cc2)c2ccccc2-c2cccc3oc4ccccc4c23)cc1 ; c1ccc(-c2ccccc2N(c2ccc(-c3cccc4c3ccc3ccccc34)cc2)c2ccccc2-c2cccc3oc4ccccc4c23)cc1]

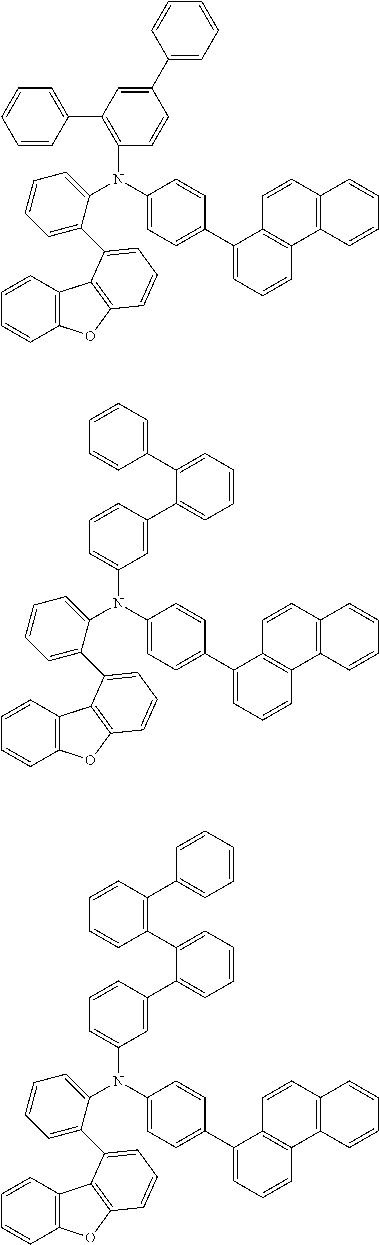 [SMILES: c1ccc(-c2ccc(N(c3ccc(-c4cccc5c4ccc4ccccc45)cc3)c3ccccc3-c3cccc4oc5ccccc5c34)c(-c3ccccc3)c2)cc1 ; c1ccc(-c2ccccc2-c2cccc(N(c3ccc(-c4cccc5c4ccc4ccccc45)cc3)c3ccccc3-c3cccc4oc5ccccc5c34)c2)cc1 ; c1ccc(-c2ccccc2-c2ccccc2-c2cccc(N(c3ccc(-c4cccc5c4ccc4ccccc45)cc3)c3ccccc3-c3cccc4oc5ccccc5c34)c2)cc1]

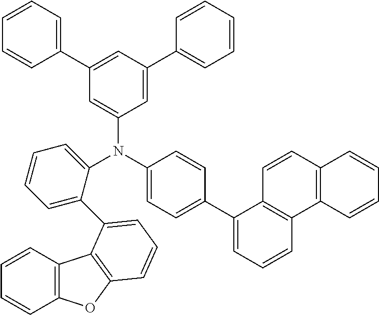 [SMILES: c1ccc(-c2cc(-c3ccccc3)cc(N(c3ccc(-c4cccc5c4ccc4ccccc45)cc3)c3ccccc3-c3cccc4oc5ccccc5c34)c2)cc1]

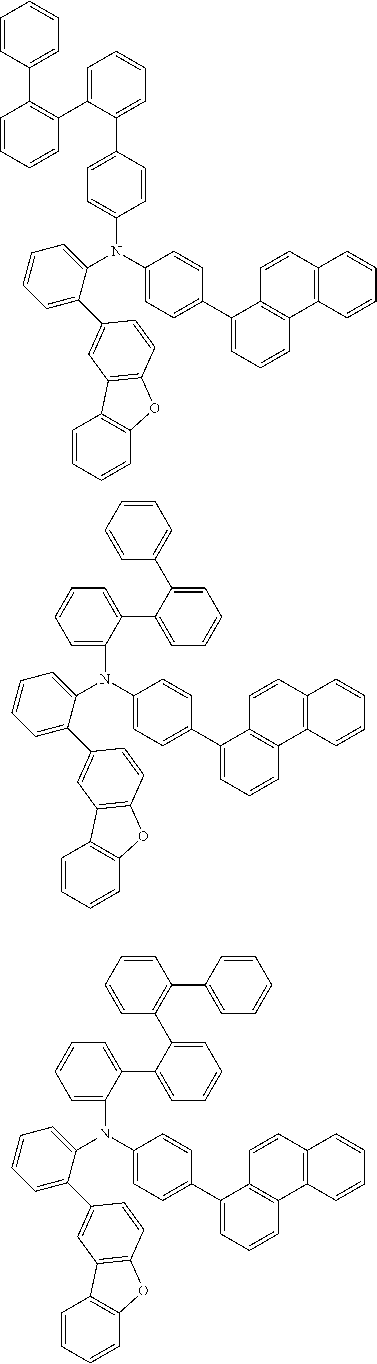

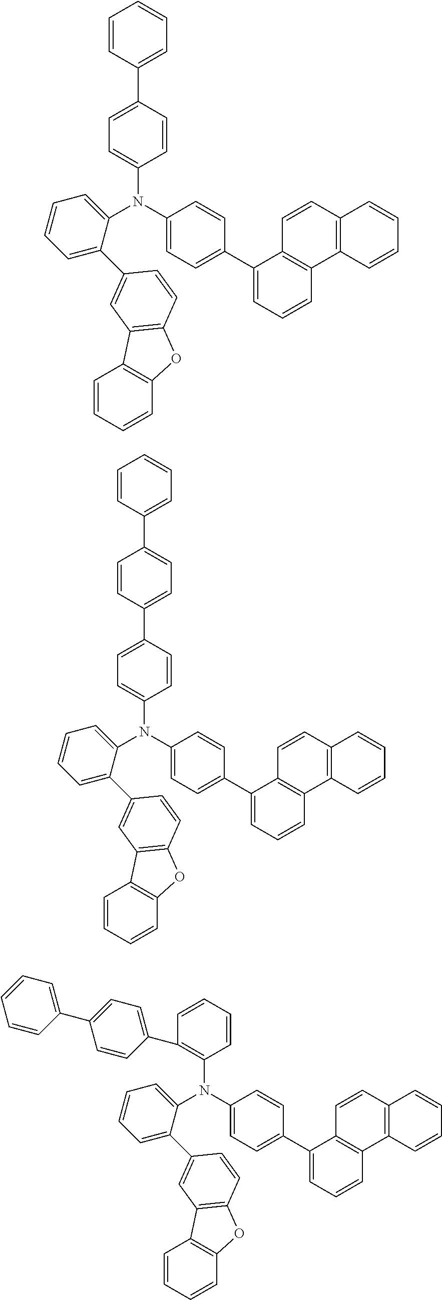

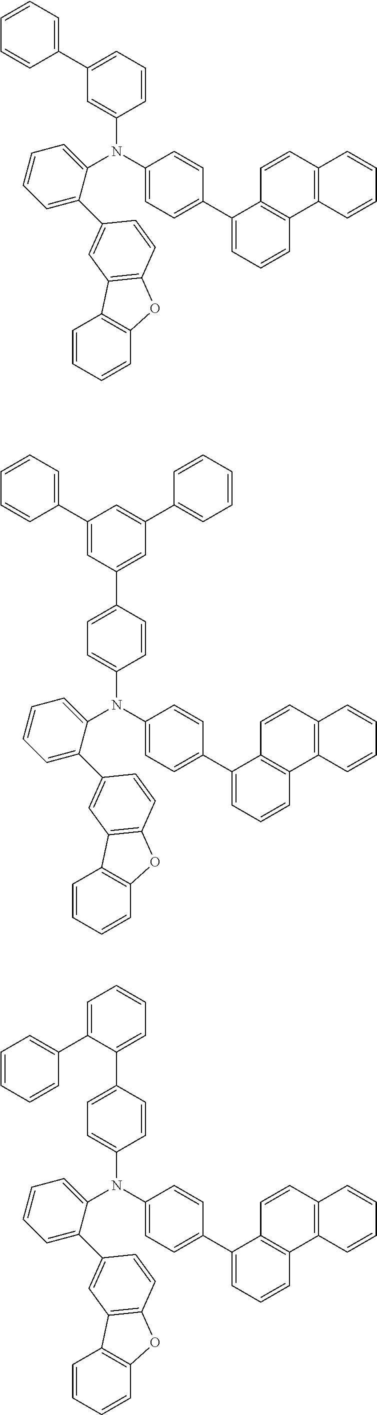 [SMILES: c1ccc(-c2cccc(N(c3ccc(-c4cccc5c4ccc4ccccc45)cc3)c3ccccc3-c3ccc4oc5ccccc5c4c3)c2)cc1 ; c1ccc(-c2cc(-c3ccccc3)cc(-c3ccc(N(c4ccc(-c5cccc6c5ccc5ccccc56)cc4)c4ccccc4-c4ccc5oc6ccccc6c5c4)cc3)c2)cc1 ; c1ccc(-c2ccccc2-c2ccc(N(c3ccc(-c4cccc5c4ccc4ccccc45)cc3)c3ccccc3-c3ccc4oc5ccccc5c4c3)cc2)cc1]

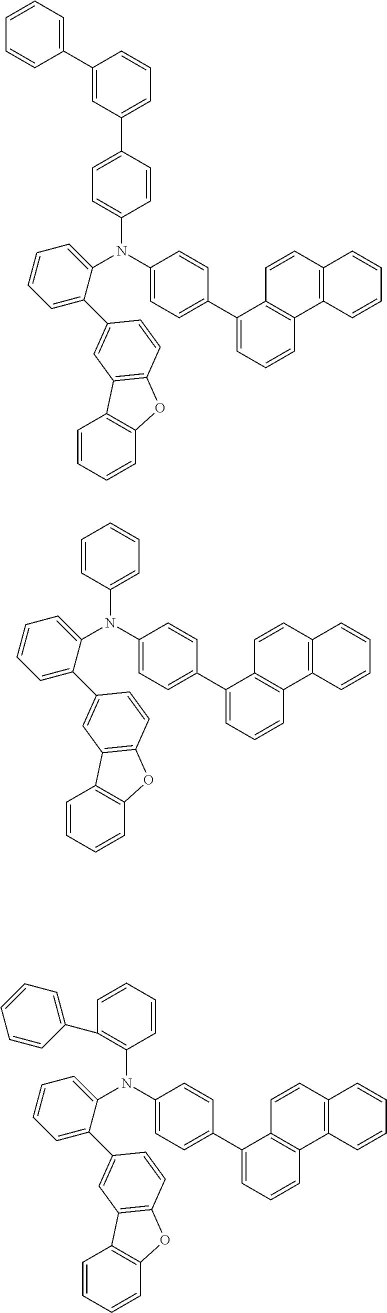

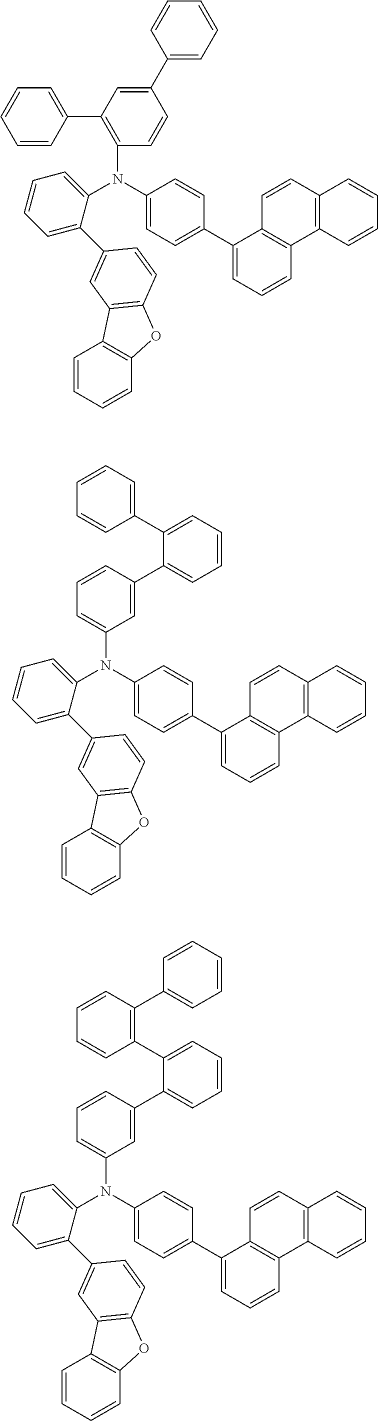 [SMILES: c1ccc(-c2ccc(N(c3ccc(-c4cccc5c4ccc4ccccc45)cc3)c3ccccc3-c3ccc4oc5ccccc5c4c3)c(-c3ccccc3)c2)cc1 ; c1ccc(-c2ccccc2-c2cccc(N(c3ccc(-c4cccc5c4ccc4ccccc45)cc3)c3ccccc3-c3ccc4oc5ccccc5c4c3)c2)cc1 ; c1ccc(-c2ccccc2-c2ccccc2-c2cccc(N(c3ccc(-c4cccc5c4ccc4ccccc45)cc3)c3ccccc3-c3ccc4oc5ccccc5c4c3)c2)cc1]

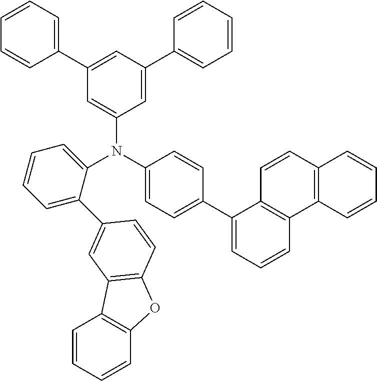 [SMILES: c1ccc(-c2cc(-c3ccccc3)cc(N(c3ccc(-c4cccc5c4ccc4ccccc45)cc3)c3ccccc3-c3ccc4oc5ccccc5c4c3)c2)cc1]

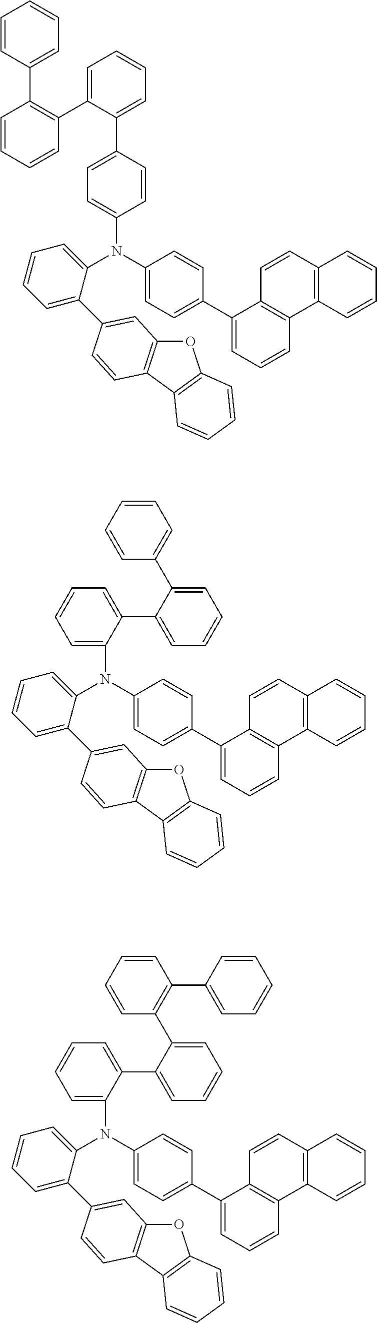 [SMILES: c1ccc(-c2ccccc2-c2ccccc2-c2ccc(N(c3ccc(-c4cccc5c4ccc4ccccc45)cc3)c3ccccc3-c3ccc4c(c3)oc3ccccc34)cc2)cc1 ; c1ccc(-c2ccccc2-c2ccccc2N(c2ccc(-c3cccc4c3ccc3ccccc34)cc2)c2ccccc2-c2ccc3c(c2)oc2ccccc23)cc1 ; c1ccc(-c2ccccc2-c2ccccc2-c2ccccc2N(c2ccc(-c3cccc4c3ccc3ccccc34)cc2)c2ccccc2-c2ccc3c(c2)oc2ccccc23)cc1]

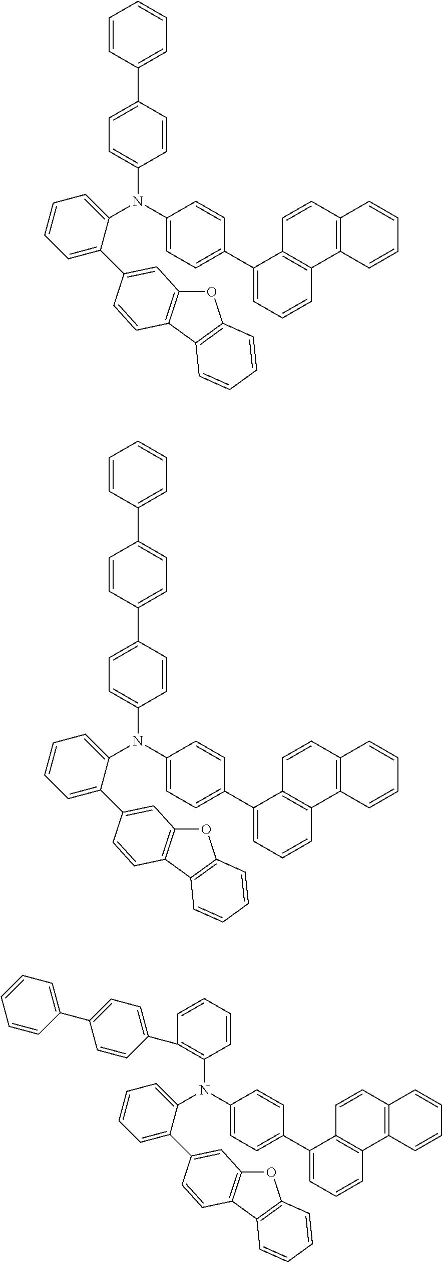 [SMILES: c1ccc(-c2ccc(N(c3ccc(-c4cccc5c4ccc4ccccc45)cc3)c3ccccc3-c3ccc4c(c3)oc3ccccc34)cc2)cc1 ; c1ccc(-c2ccc(-c3ccc(N(c4ccc(-c5cccc6c5ccc5ccccc56)cc4)c4ccccc4-c4ccc5c(c4)oc4ccccc45)cc3)cc2)cc1 ; c1ccc(-c2ccc(-c3ccccc3N(c3ccc(-c4cccc5c4ccc4ccccc45)cc3)c3ccccc3-c3ccc4c(c3)oc3ccccc34)cc2)cc1]

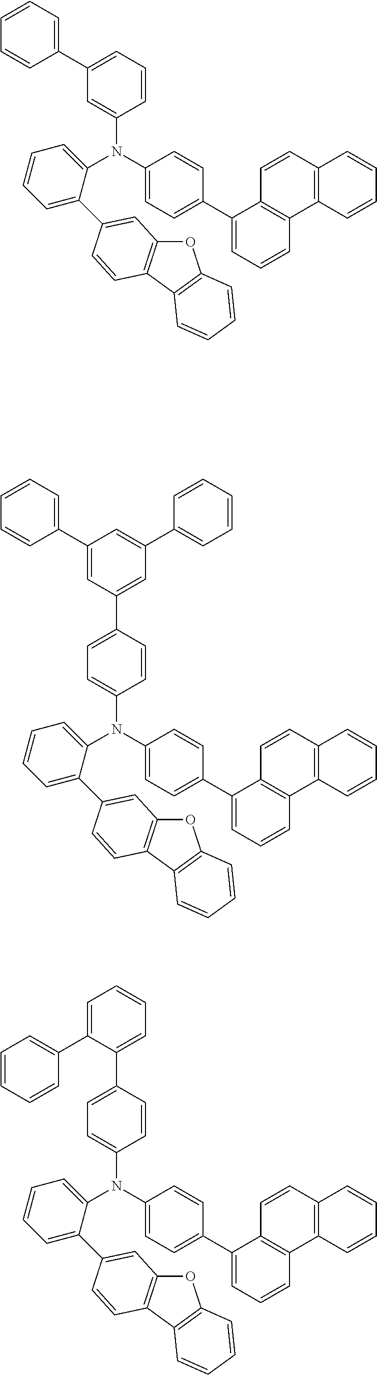 [SMILES: c1ccc(-c2cccc(N(c3ccc(-c4cccc5c4ccc4ccccc45)cc3)c3ccccc3-c3ccc4c(c3)oc3ccccc34)c2)cc1 ; c1ccc(-c2cc(-c3ccccc3)cc(-c3ccc(N(c4ccc(-c5cccc6c5ccc5ccccc56)cc4)c4ccccc4-c4ccc5c(c4)oc4ccccc45)cc3)c2)cc1 ; c1ccc(-c2ccccc2-c2ccc(N(c3ccc(-c4cccc5c4ccc4ccccc45)cc3)c3ccccc3-c3ccc4c(c3)oc3ccccc34)cc2)cc1]

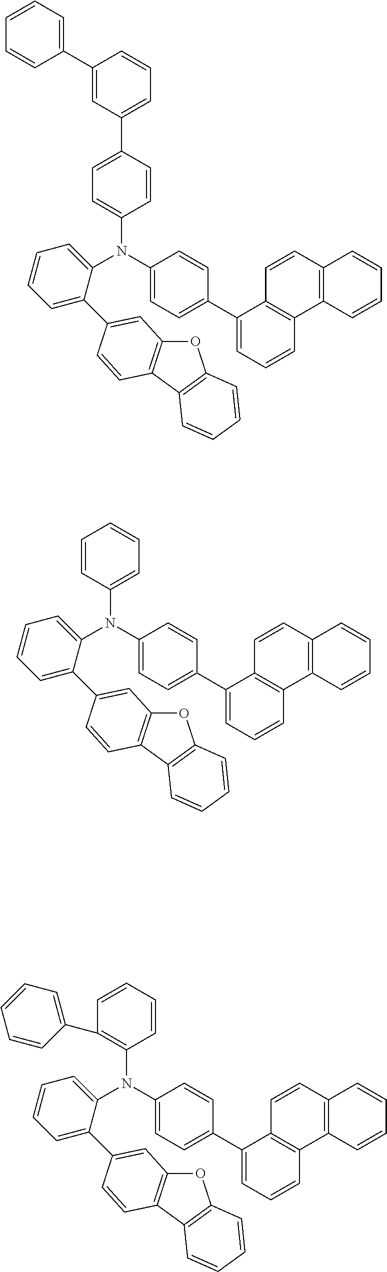 [SMILES: c1ccc(-c2cccc(-c3ccc(N(c4ccc(-c5cccc6c5ccc5ccccc56)cc4)c4ccccc4-c4ccc5c(c4)oc4ccccc45)cc3)c2)cc1 ; c1ccc(N(c2ccc(-c3cccc4c3ccc3ccccc34)cc2)c2ccccc2-c2ccc3c(c2)oc2ccccc23)cc1 ; c1ccc(-c2ccccc2N(c2ccc(-c3cccc4c3ccc3ccccc34)cc2)c2ccccc2-c2ccc3c(c2)oc2ccccc23)cc1]

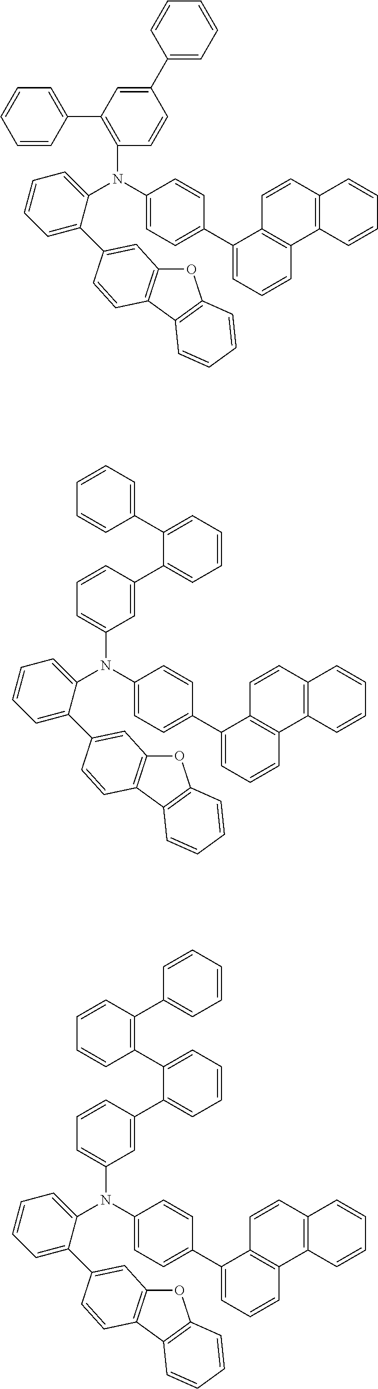 [SMILES: c1ccc(-c2ccc(N(c3ccc(-c4cccc5c4ccc4ccccc45)cc3)c3ccccc3-c3ccc4c(c3)oc3ccccc34)c(-c3ccccc3)c2)cc1 ; c1ccc(-c2ccccc2-c2cccc(N(c3ccc(-c4cccc5c4ccc4ccccc45)cc3)c3ccccc3-c3ccc4c(c3)oc3ccccc34)c2)cc1 ; c1ccc(-c2ccccc2-c2ccccc2-c2cccc(N(c3ccc(-c4cccc5c4ccc4ccccc45)cc3)c3ccccc3-c3ccc4c(c3)oc3ccccc34)c2)cc1]

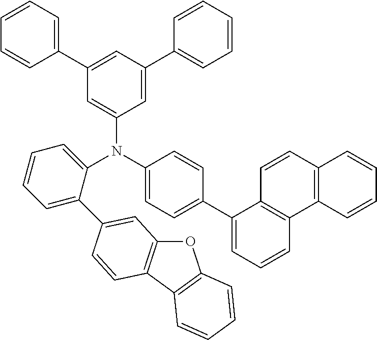 [SMILES: c1ccc(-c2cc(-c3ccccc3)cc(N(c3ccc(-c4cccc5c4ccc4ccccc45)cc3)c3ccccc3-c3ccc4c(c3)oc3ccccc34)c2)cc1]

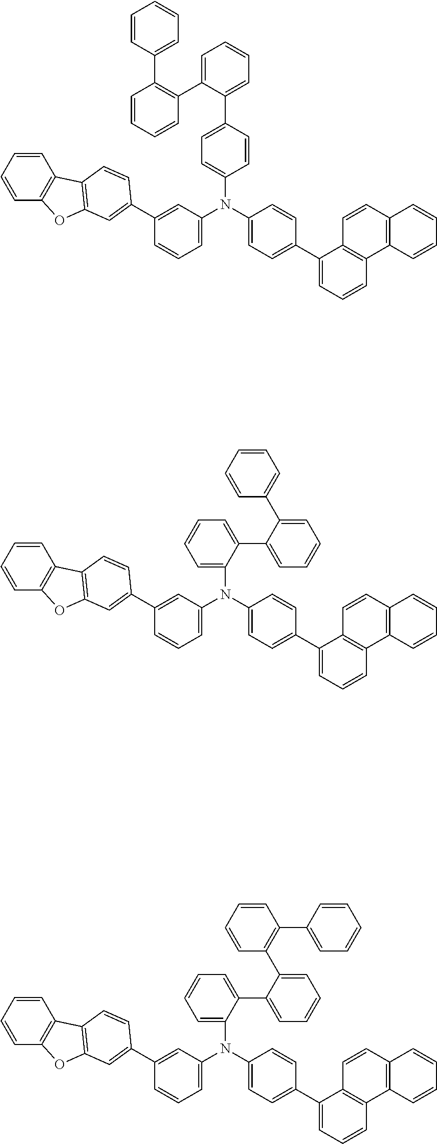 [SMILES: c1ccc(-c2ccccc2-c2ccccc2-c2ccc(N(c3ccc(-c4cccc5c4ccc4ccccc45)cc3)c3cccc(-c4ccc5c(c4)oc4ccccc45)c3)cc2)cc1 ; c1ccc(-c2ccccc2-c2ccccc2N(c2ccc(-c3cccc4c3ccc3ccccc34)cc2)c2cccc(-c3ccc4c(c3)oc3ccccc34)c2)cc1 ; c1ccc(-c2ccccc2-c2ccccc2-c2ccccc2N(c2ccc(-c3cccc4c3ccc3ccccc34)cc2)c2cccc(-c3ccc4c(c3)oc3ccccc34)c2)cc1]

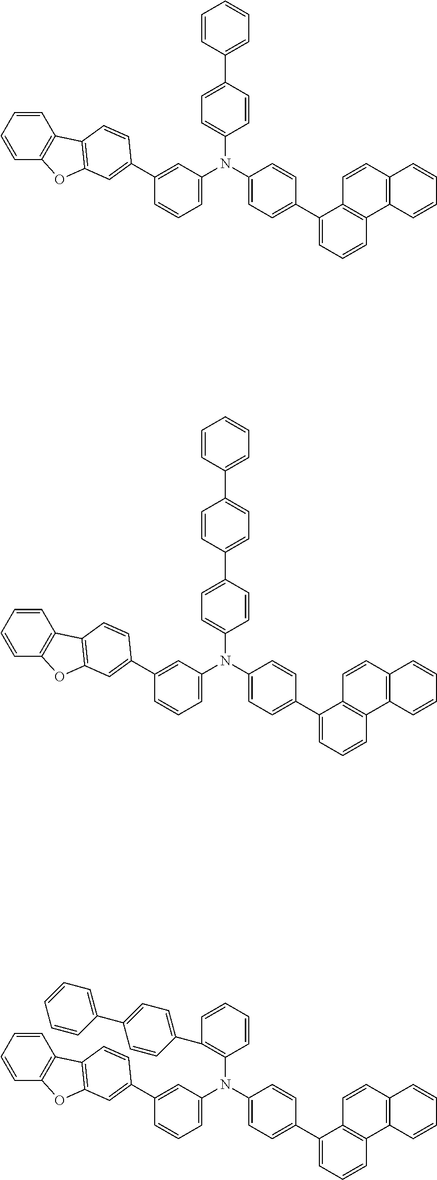 [SMILES: c1ccc(-c2ccc(N(c3ccc(-c4cccc5c4ccc4ccccc45)cc3)c3cccc(-c4ccc5c(c4)oc4ccccc45)c3)cc2)cc1 ; c1ccc(-c2ccc(-c3ccc(N(c4ccc(-c5cccc6c5ccc5ccccc56)cc4)c4cccc(-c5ccc6c(c5)oc5ccccc56)c4)cc3)cc2)cc1 ; c1ccc(-c2ccc(-c3ccccc3N(c3ccc(-c4cccc5c4ccc4ccccc45)cc3)c3cccc(-c4ccc5c(c4)oc4ccccc45)c3)cc2)cc1]

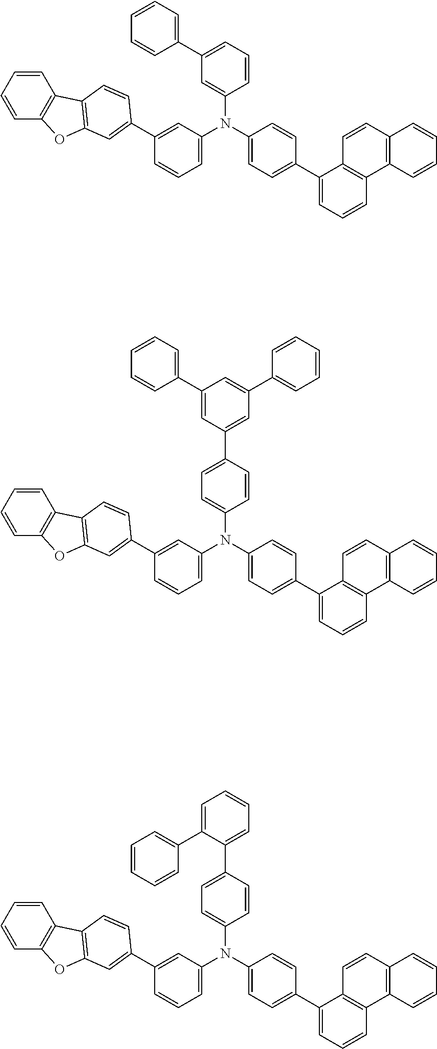 [SMILES: c1ccc(-c2cccc(N(c3ccc(-c4cccc5c4ccc4ccccc45)cc3)c3cccc(-c4ccc5c(c4)oc4ccccc45)c3)c2)cc1 ; c1ccc(-c2cc(-c3ccccc3)cc(-c3ccc(N(c4ccc(-c5cccc6c5ccc5ccccc56)cc4)c4cccc(-c5ccc6c(c5)oc5ccccc56)c4)cc3)c2)cc1 ; c1ccc(-c2ccccc2-c2ccc(N(c3ccc(-c4cccc5c4ccc4ccccc45)cc3)c3cccc(-c4ccc5c(c4)oc4ccccc45)c3)cc2)cc1]

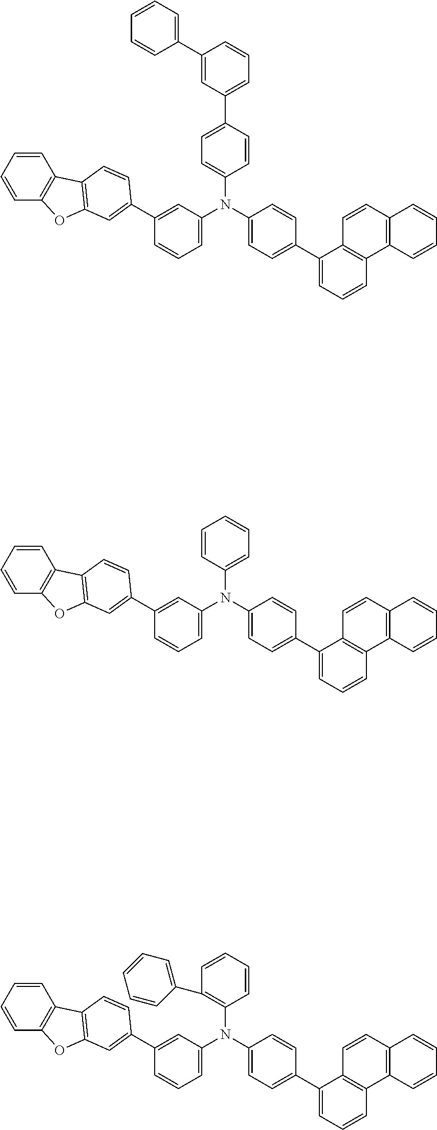 [SMILES: c1ccc(-c2cccc(-c3ccc(N(c4ccc(-c5cccc6c5ccc5ccccc56)cc4)c4cccc(-c5ccc6c(c5)oc5ccccc56)c4)cc3)c2)cc1 ; c1ccc(N(c2ccc(-c3cccc4c3ccc3ccccc34)cc2)c2cccc(-c3ccc4c(c3)oc3ccccc34)c2)cc1 ; c1ccc(-c2ccccc2N(c2ccc(-c3cccc4c3ccc3ccccc34)cc2)c2cccc(-c3ccc4c(c3)oc3ccccc34)c2)cc1]

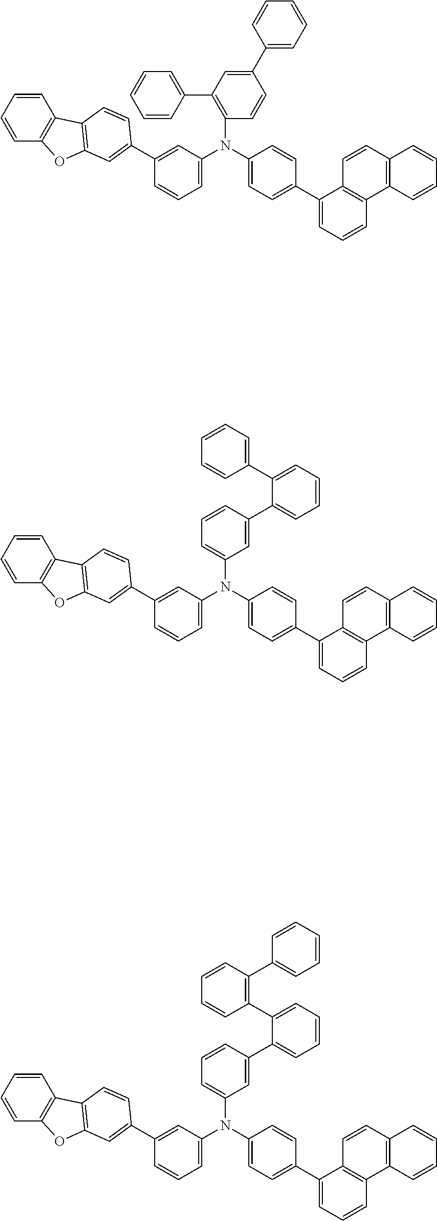 [SMILES: c1ccc(-c2ccc(N(c3ccc(-c4cccc5c4ccc4ccccc45)cc3)c3cccc(-c4ccc5c(c4)oc4ccccc45)c3)c(-c3ccccc3)c2)cc1 ; c1ccc(-c2ccccc2-c2cccc(N(c3ccc(-c4cccc5c4ccc4ccccc45)cc3)c3cccc(-c4ccc5c(c4)oc4ccccc45)c3)c2)cc1 ; c1ccc(-c2ccccc2-c2ccccc2-c2cccc(N(c3ccc(-c4cccc5c4ccc4ccccc45)cc3)c3cccc(-c4ccc5c(c4)oc4ccccc45)c3)c2)cc1]

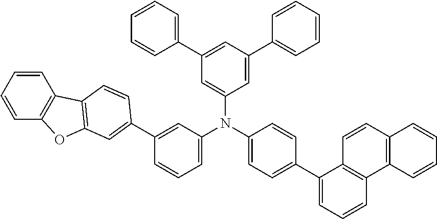 [SMILES: c1ccc(-c2cc(-c3ccccc3)cc(N(c3ccc(-c4cccc5c4ccc4ccccc45)cc3)c3cccc(-c4ccc5c(c4)oc4ccccc45)c3)c2)cc1]

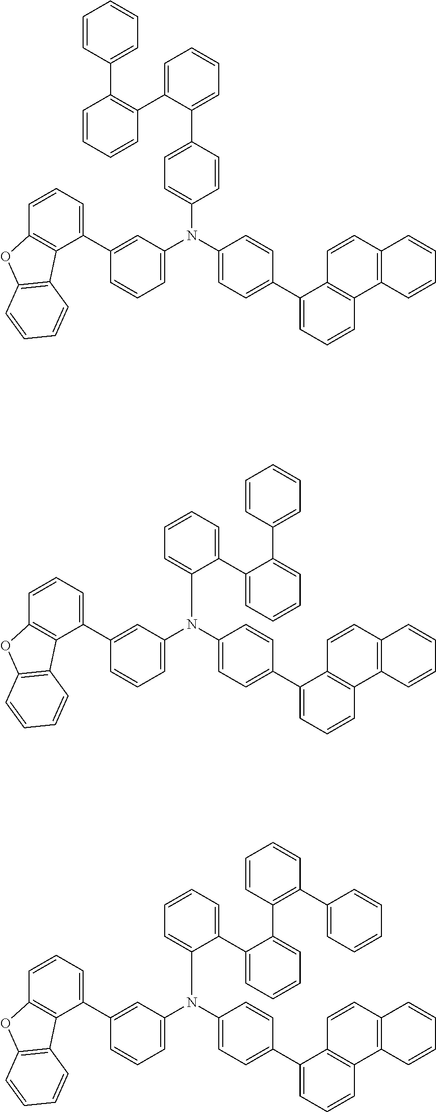 [SMILES: c1ccc(-c2ccccc2-c2ccccc2-c2ccc(N(c3ccc(-c4cccc5c4ccc4ccccc45)cc3)c3cccc(-c4cccc5oc6ccccc6c45)c3)cc2)cc1 ; c1ccc(-c2ccccc2-c2ccccc2N(c2ccc(-c3cccc4c3ccc3ccccc34)cc2)c2cccc(-c3cccc4oc5ccccc5c34)c2)cc1 ; c1ccc(-c2ccccc2-c2ccccc2-c2ccccc2N(c2ccc(-c3cccc4c3ccc3ccccc34)cc2)c2cccc(-c3cccc4oc5ccccc5c34)c2)cc1]

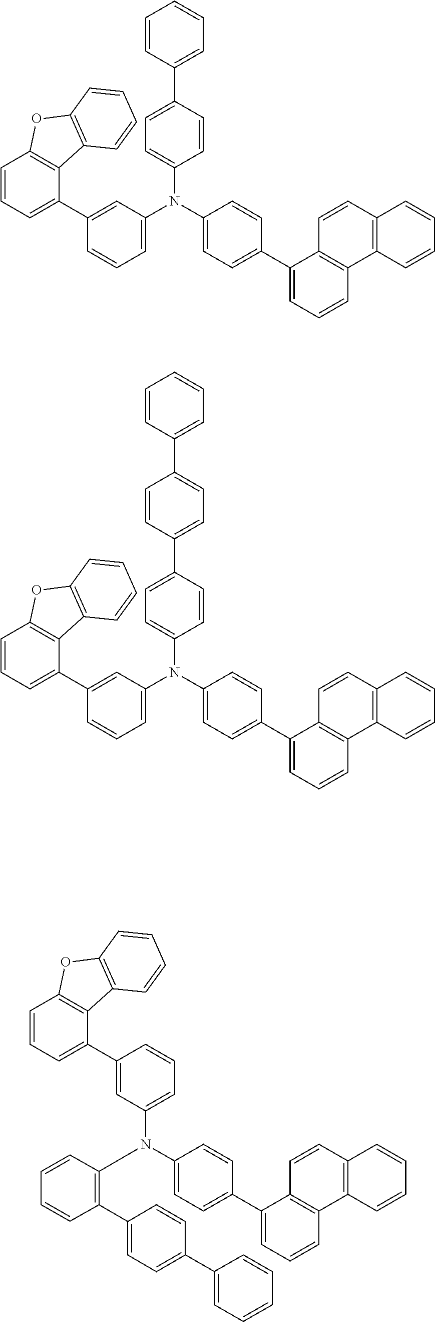 [SMILES: c1ccc(-c2ccc(N(c3ccc(-c4cccc5c4ccc4ccccc45)cc3)c3cccc(-c4cccc5oc6ccccc6c45)c3)cc2)cc1 ; c1ccc(-c2ccc(-c3ccc(N(c4ccc(-c5cccc6c5ccc5ccccc56)cc4)c4cccc(-c5cccc6oc7ccccc7c56)c4)cc3)cc2)cc1 ; c1ccc(-c2ccc(-c3ccccc3N(c3ccc(-c4cccc5c4ccc4ccccc45)cc3)c3cccc(-c4cccc5oc6ccccc6c45)c3)cc2)cc1]

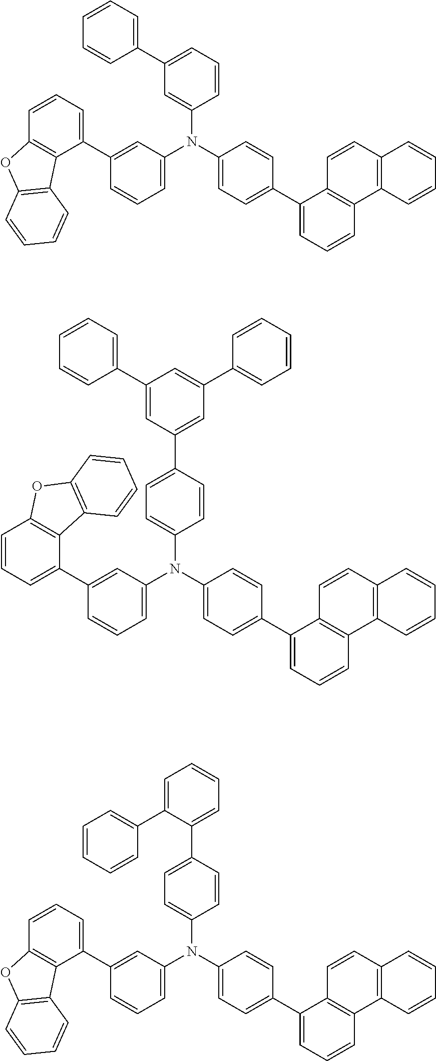 [SMILES: c1ccc(-c2cccc(N(c3ccc(-c4cccc5c4ccc4ccccc45)cc3)c3cccc(-c4cccc5oc6ccccc6c45)c3)c2)cc1 ; c1ccc(-c2cc(-c3ccccc3)cc(-c3ccc(N(c4ccc(-c5cccc6c5ccc5ccccc56)cc4)c4cccc(-c5cccc6oc7ccccc7c56)c4)cc3)c2)cc1 ; c1ccc(-c2ccccc2-c2ccc(N(c3ccc(-c4cccc5c4ccc4ccccc45)cc3)c3cccc(-c4cccc5oc6ccccc6c45)c3)cc2)cc1]

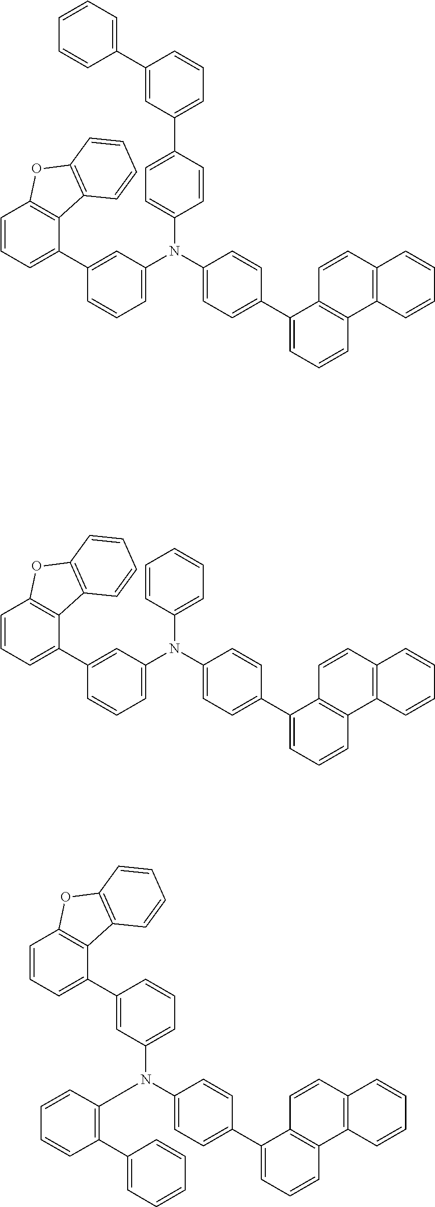 [SMILES: c1ccc(-c2cccc(-c3ccc(N(c4ccc(-c5cccc6c5ccc5ccccc56)cc4)c4cccc(-c5cccc6oc7ccccc7c56)c4)cc3)c2)cc1 ; c1ccc(N(c2ccc(-c3cccc4c3ccc3ccccc34)cc2)c2cccc(-c3cccc4oc5ccccc5c34)c2)cc1 ; c1ccc(-c2ccccc2N(c2ccc(-c3cccc4c3ccc3ccccc34)cc2)c2cccc(-c3cccc4oc5ccccc5c34)c2)cc1]

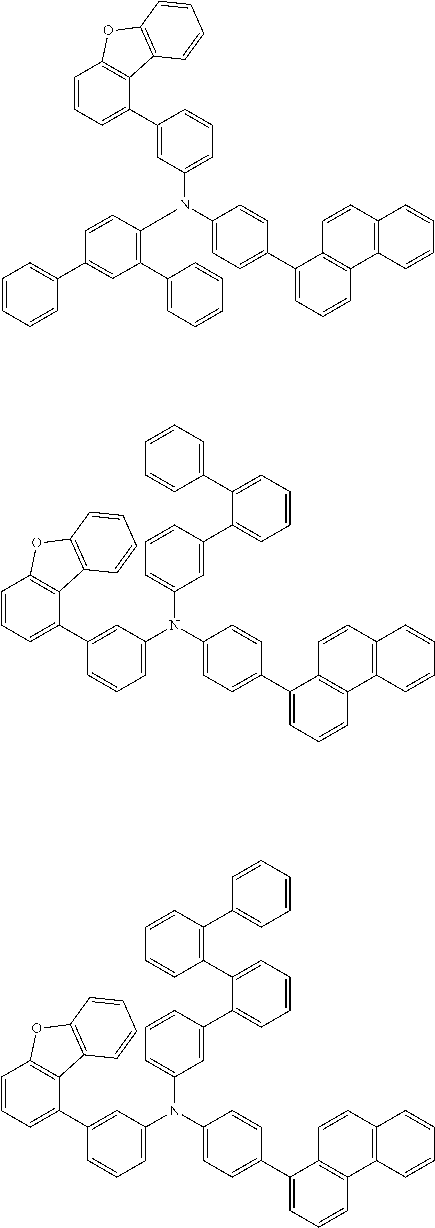 [SMILES: c1ccc(-c2ccc(N(c3ccc(-c4cccc5c4ccc4ccccc45)cc3)c3cccc(-c4cccc5oc6ccccc6c45)c3)c(-c3ccccc3)c2)cc1 ; c1ccc(-c2ccccc2-c2cccc(N(c3ccc(-c4cccc5c4ccc4ccccc45)cc3)c3cccc(-c4cccc5oc6ccccc6c45)c3)c2)cc1 ; c1ccc(-c2ccccc2-c2ccccc2-c2cccc(N(c3ccc(-c4cccc5c4ccc4ccccc45)cc3)c3cccc(-c4cccc5oc6ccccc6c45)c3)c2)cc1]

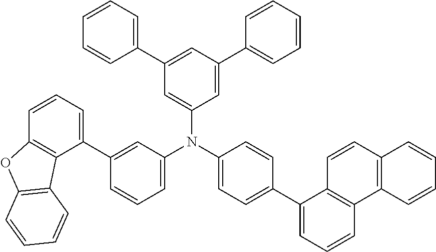 [SMILES: c1ccc(-c2cc(-c3ccccc3)cc(N(c3ccc(-c4cccc5c4ccc4ccccc45)cc3)c3cccc(-c4cccc5oc6ccccc6c45)c3)c2)cc1]

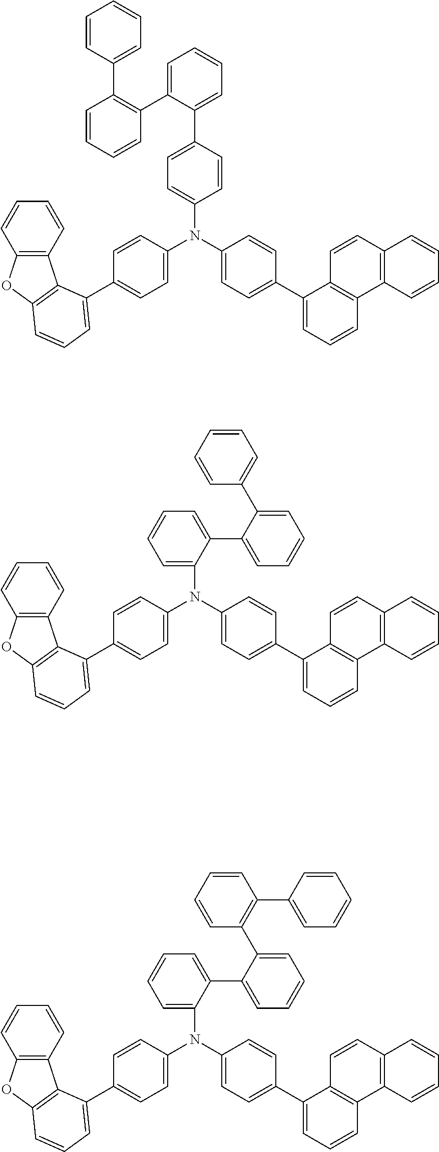 [SMILES: c1ccc(-c2ccccc2-c2ccccc2-c2ccc(N(c3ccc(-c4cccc5c4ccc4ccccc45)cc3)c3ccc(-c4cccc5oc6ccccc6c45)cc3)cc2)cc1 ; c1ccc(-c2ccccc2-c2ccccc2N(c2ccc(-c3cccc4c3ccc3ccccc34)cc2)c2ccc(-c3cccc4oc5ccccc5c34)cc2)cc1 ; c1ccc(-c2ccccc2-c2ccccc2-c2ccccc2N(c2ccc(-c3cccc4c3ccc3ccccc34)cc2)c2ccc(-c3cccc4oc5ccccc5c34)cc2)cc1]

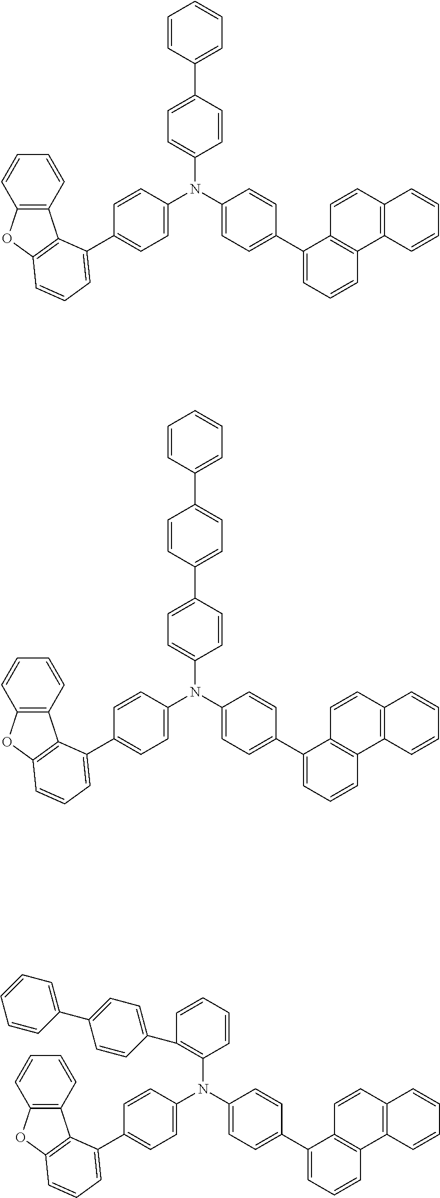 [SMILES: c1ccc(-c2ccc(N(c3ccc(-c4cccc5c4ccc4ccccc45)cc3)c3ccc(-c4cccc5oc6ccccc6c45)cc3)cc2)cc1 ; c1ccc(-c2ccc(-c3ccc(N(c4ccc(-c5cccc6c5ccc5ccccc56)cc4)c4ccc(-c5cccc6oc7ccccc7c56)cc4)cc3)cc2)cc1 ; c1ccc(-c2ccc(-c3ccccc3N(c3ccc(-c4cccc5c4ccc4ccccc45)cc3)c3ccc(-c4cccc5oc6ccccc6c45)cc3)cc2)cc1]

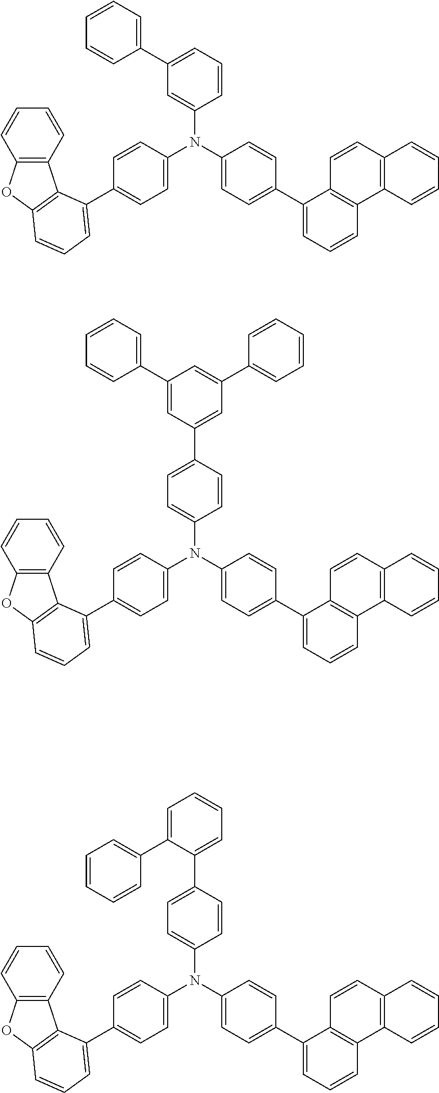 [SMILES: c1ccc(-c2cccc(N(c3ccc(-c4cccc5c4ccc4ccccc45)cc3)c3ccc(-c4cccc5oc6ccccc6c45)cc3)c2)cc1 ; c1ccc(-c2cc(-c3ccccc3)cc(-c3ccc(N(c4ccc(-c5cccc6c5ccc5ccccc56)cc4)c4ccc(-c5cccc6oc7ccccc7c56)cc4)cc3)c2)cc1 ; c1ccc(-c2ccccc2-c2ccc(N(c3ccc(-c4cccc5c4ccc4ccccc45)cc3)c3ccc(-c4cccc5oc6ccccc6c45)cc3)cc2)cc1]

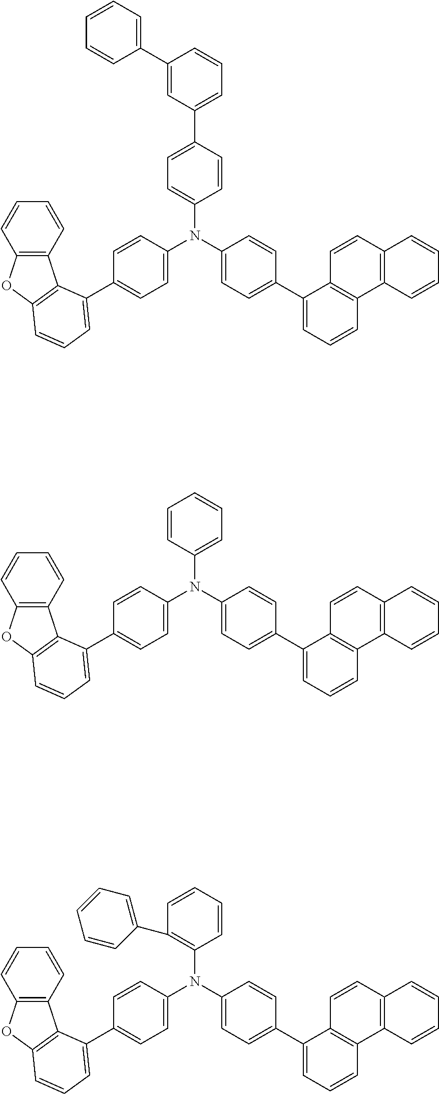 [SMILES: c1ccc(-c2cccc(-c3ccc(N(c4ccc(-c5cccc6c5ccc5ccccc56)cc4)c4ccc(-c5cccc6oc7ccccc7c56)cc4)cc3)c2)cc1 ; c1ccc(N(c2ccc(-c3cccc4c3ccc3ccccc34)cc2)c2ccc(-c3cccc4oc5ccccc5c34)cc2)cc1 ; c1ccc(-c2ccccc2N(c2ccc(-c3cccc4c3ccc3ccccc34)cc2)c2ccc(-c3cccc4oc5ccccc5c34)cc2)cc1]

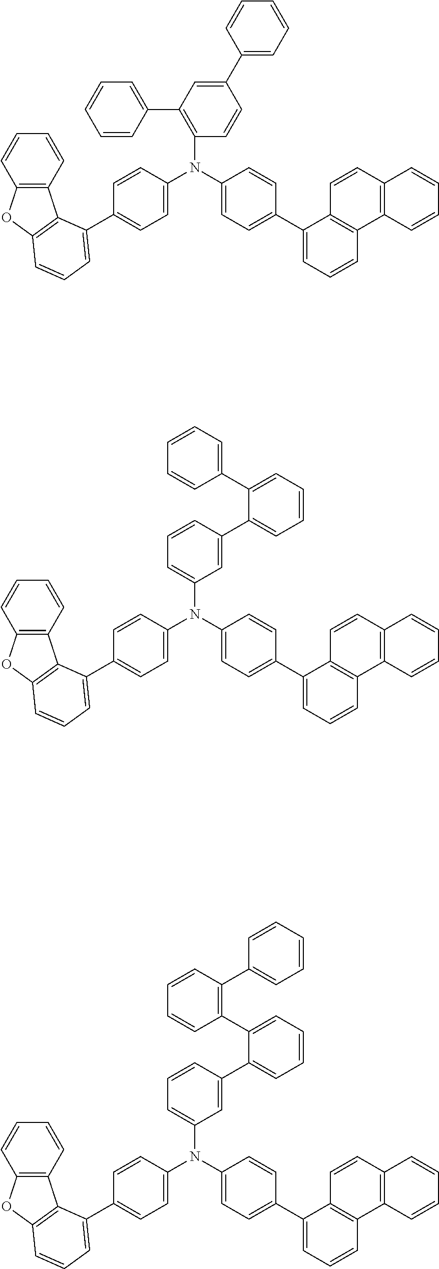 [SMILES: c1ccc(-c2ccc(N(c3ccc(-c4cccc5c4ccc4ccccc45)cc3)c3ccc(-c4cccc5oc6ccccc6c45)cc3)c(-c3ccccc3)c2)cc1 ; c1ccc(-c2ccccc2-c2cccc(N(c3ccc(-c4cccc5c4ccc4ccccc45)cc3)c3ccc(-c4cccc5oc6ccccc6c45)cc3)c2)cc1 ; c1ccc(-c2ccccc2-c2ccccc2-c2cccc(N(c3ccc(-c4cccc5c4ccc4ccccc45)cc3)c3ccc(-c4cccc5oc6ccccc6c45)cc3)c2)cc1]

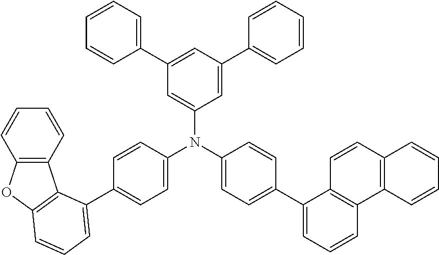 [SMILES: c1ccc(-c2cc(-c3ccccc3)cc(N(c3ccc(-c4cccc5c4ccc4ccccc45)cc3)c3ccc(-c4cccc5oc6ccccc6c45)cc3)c2)cc1]

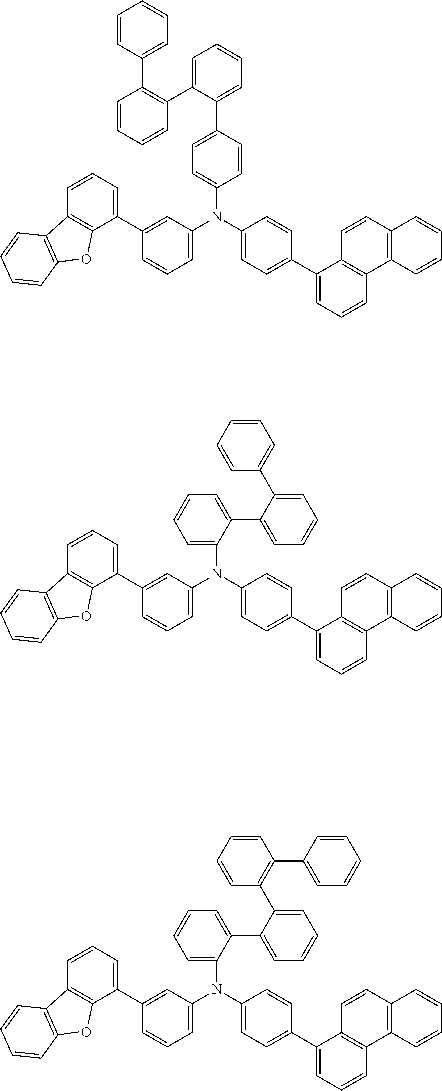 [SMILES: c1ccc(-c2ccccc2-c2ccccc2-c2ccc(N(c3ccc(-c4cccc5c4ccc4ccccc45)cc3)c3cccc(-c4cccc5c4oc4ccccc45)c3)cc2)cc1 ; c1ccc(-c2ccccc2-c2ccccc2N(c2ccc(-c3cccc4c3ccc3ccccc34)cc2)c2cccc(-c3cccc4c3oc3ccccc34)c2)cc1 ; c1ccc(-c2ccccc2-c2ccccc2-c2ccccc2N(c2ccc(-c3cccc4c3ccc3ccccc34)cc2)c2cccc(-c3cccc4c3oc3ccccc34)c2)cc1]

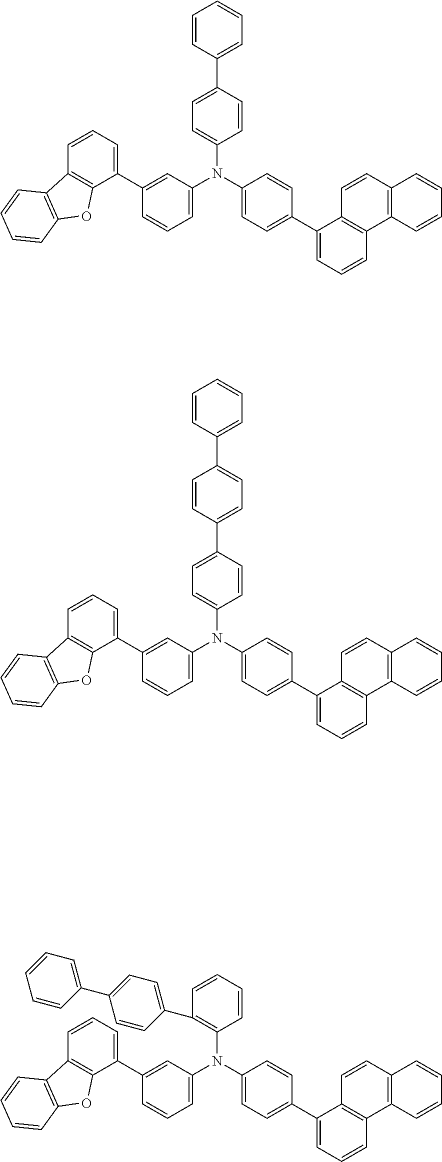 [SMILES: c1ccc(-c2ccc(N(c3ccc(-c4cccc5c4ccc4ccccc45)cc3)c3cccc(-c4cccc5c4oc4ccccc45)c3)cc2)cc1 ; c1ccc(-c2ccc(-c3ccc(N(c4ccc(-c5cccc6c5ccc5ccccc56)cc4)c4cccc(-c5cccc6c5oc5ccccc56)c4)cc3)cc2)cc1 ; c1ccc(-c2ccc(-c3ccccc3N(c3ccc(-c4cccc5c4ccc4ccccc45)cc3)c3cccc(-c4cccc5c4oc4ccccc45)c3)cc2)cc1]

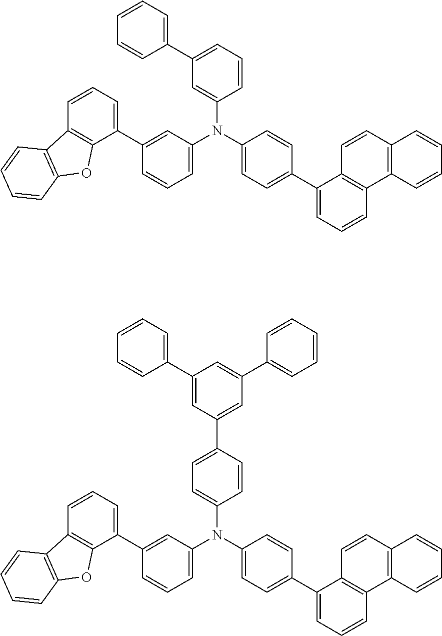 [SMILES: c1ccc(-c2cccc(N(c3ccc(-c4cccc5c4ccc4ccccc45)cc3)c3cccc(-c4cccc5c4oc4ccccc45)c3)c2)cc1 ; c1ccc(-c2cc(-c3ccccc3)cc(-c3ccc(N(c4ccc(-c5cccc6c5ccc5ccccc56)cc4)c4cccc(-c5cccc6c5oc5ccccc56)c4)cc3)c2)cc1]

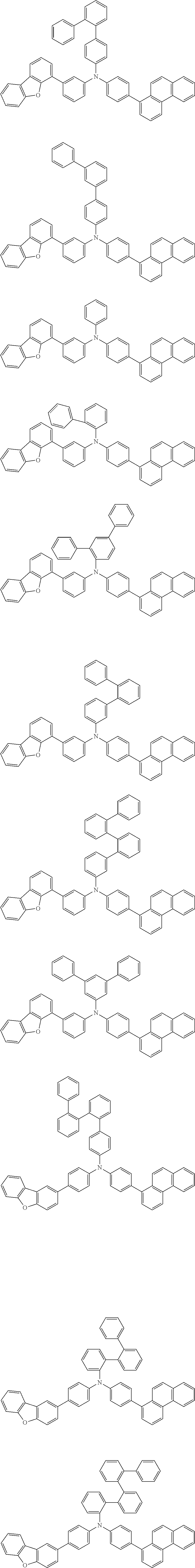

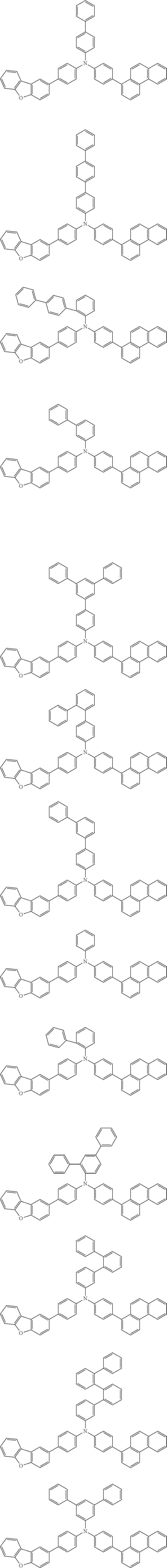

[chemical structure]

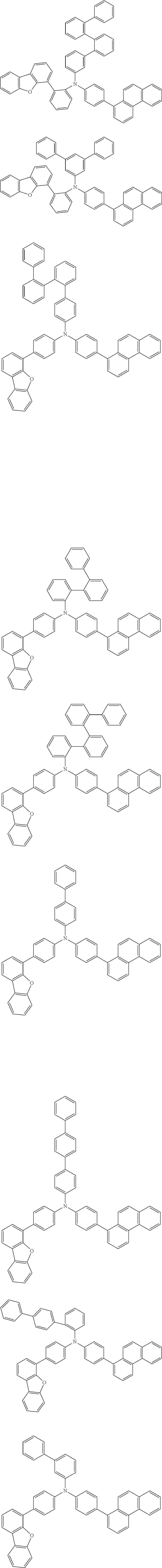

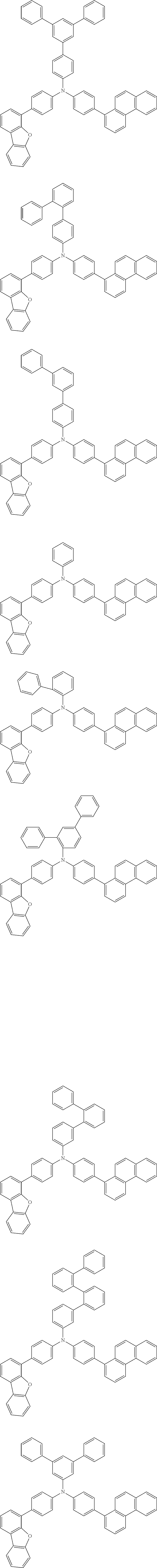

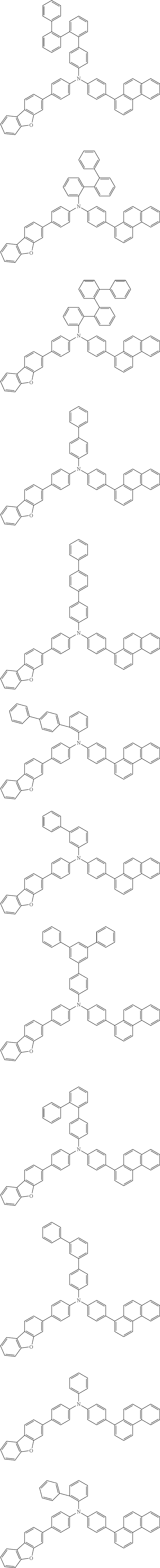

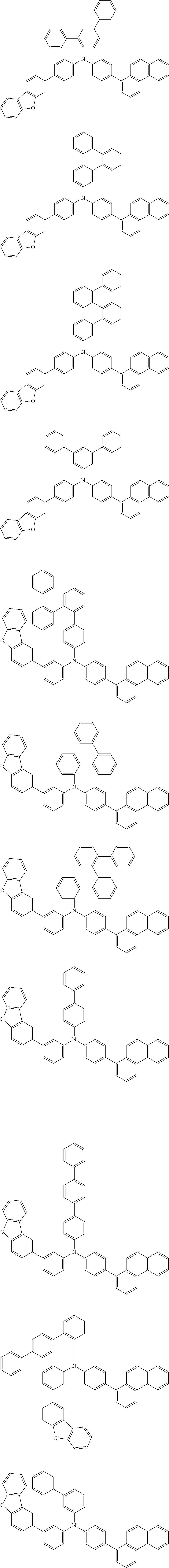

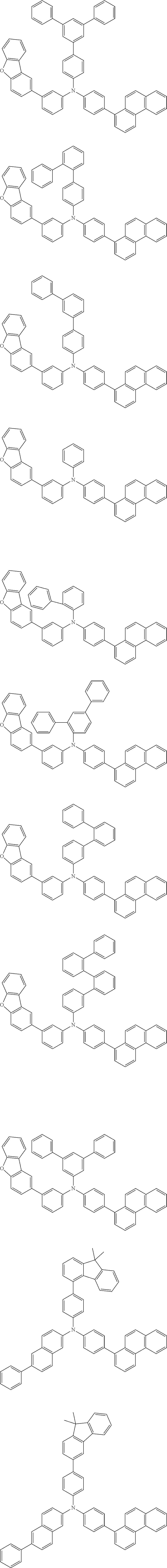

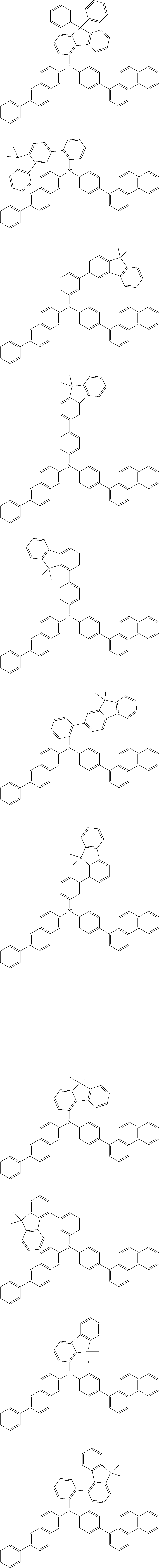

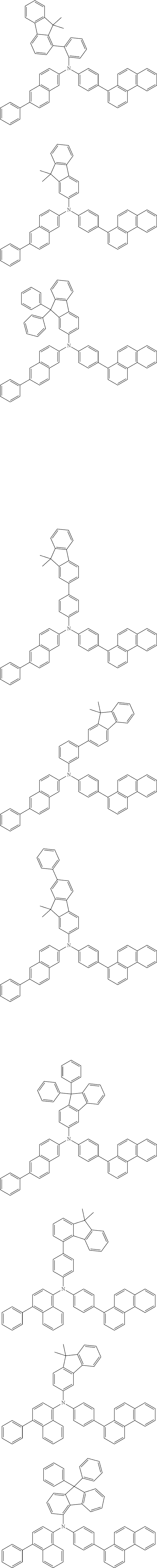

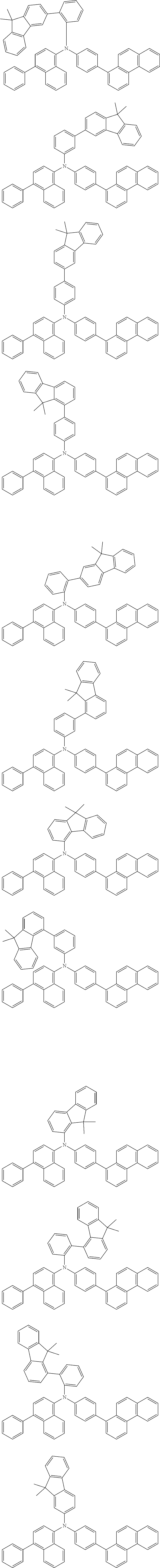

[chemical structure]

[chemical structure]

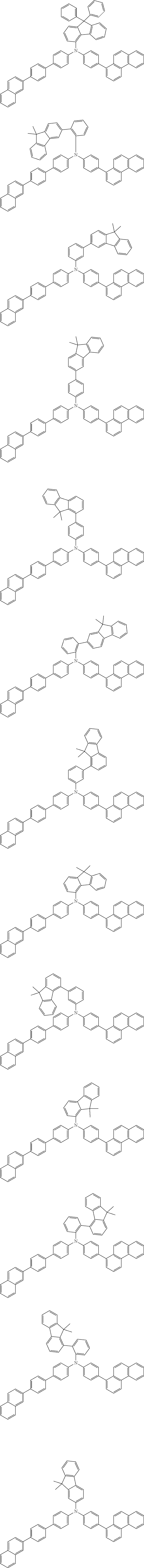

[chemical structure]

[chemical structure]

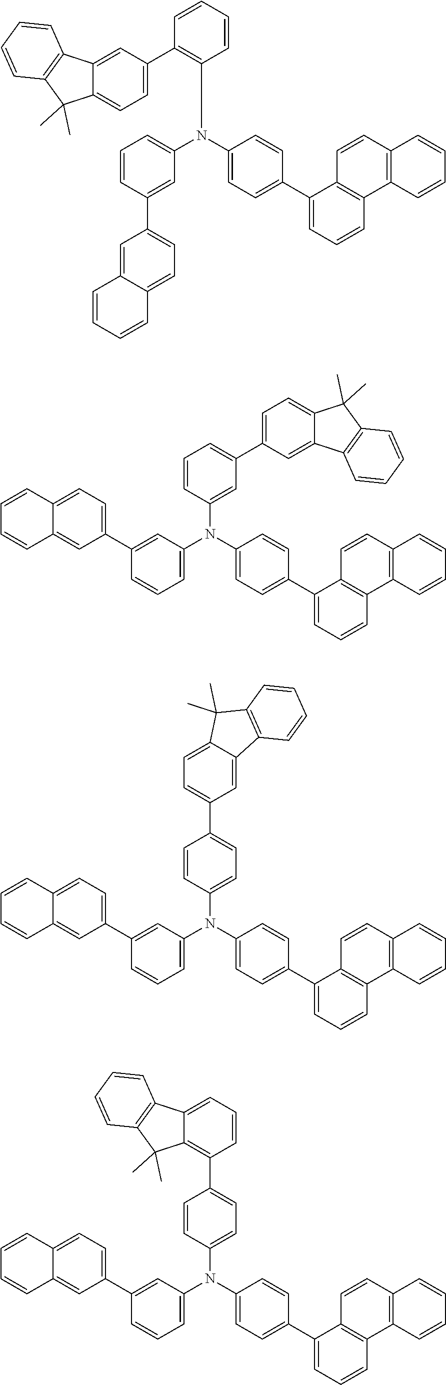 [SMILES: CC1(C)c2ccccc2-c2cc(-c3ccccc3N(c3ccc(-c4cccc5c4ccc4ccccc45)cc3)c3cccc(-c4ccc5ccccc5c4)c3)ccc21 ; CC1(C)c2ccccc2-c2cc(-c3cccc(N(c4ccc(-c5cccc6c5ccc5ccccc56)cc4)c4cccc(-c5ccc6ccccc6c5)c4)c3)ccc21 ; CC1(C)c2ccccc2-c2cc(-c3ccc(N(c4ccc(-c5cccc6c5ccc5ccccc56)cc4)c4cccc(-c5ccc6ccccc6c5)c4)cc3)ccc21 ; CC1(C)c2ccccc2-c2cccc(-c3ccc(N(c4ccc(-c5cccc6c5ccc5ccccc56)cc4)c4cccc(-c5ccc6ccccc6c5)c4)cc3)c21]

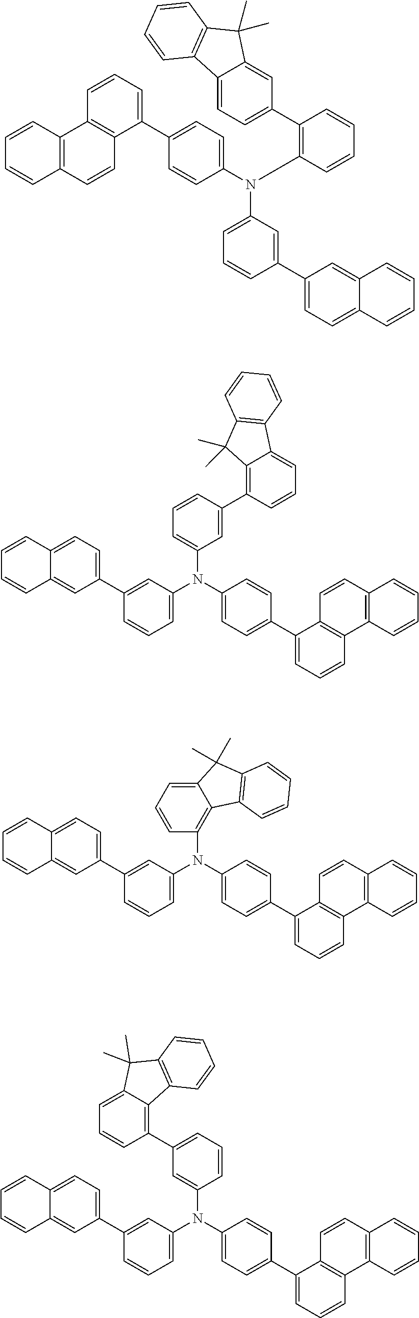 [SMILES: CC1(C)c2ccccc2-c2ccc(-c3ccccc3N(c3ccc(-c4cccc5c4ccc4ccccc45)cc3)c3cccc(-c4ccc5ccccc5c4)c3)cc21 ; CC1(C)c2ccccc2-c2cccc(-c3cccc(N(c4ccc(-c5cccc6c5ccc5ccccc56)cc4)c4cccc(-c5ccc6ccccc6c5)c4)c3)c21 ; CC1(C)c2ccccc2-c2c(N(c3ccc(-c4cccc5c4ccc4ccccc45)cc3)c3cccc(-c4ccc5ccccc5c4)c3)cccc21 ; CC1(C)c2ccccc2-c2c(-c3cccc(N(c4ccc(-c5cccc6c5ccc5ccccc56)cc4)c4cccc(-c5ccc6ccccc6c5)c4)c3)cccc21]

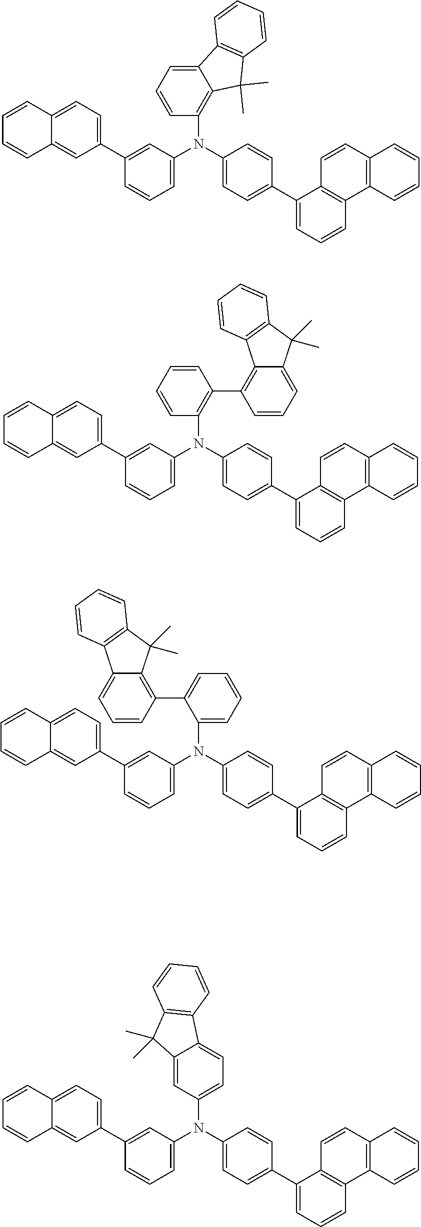 [SMILES: CC1(C)c2ccccc2-c2cccc(N(c3ccc(-c4cccc5c4ccc4ccccc45)cc3)c3cccc(-c4ccc5ccccc5c4)c3)c21 ; CC1(C)c2ccccc2-c2c(-c3ccccc3N(c3ccc(-c4cccc5c4ccc4ccccc45)cc3)c3cccc(-c4ccc5ccccc5c4)c3)cccc21 ; CC1(C)c2ccccc2-c2cccc(-c3ccccc3N(c3ccc(-c4cccc5c4ccc4ccccc45)cc3)c3cccc(-c4ccc5ccccc5c4)c3)c21 ; CC1(C)c2ccccc2-c2ccc(N(c3ccc(-c4cccc5c4ccc4ccccc45)cc3)c3cccc(-c4ccc5ccccc5c4)c3)cc21]

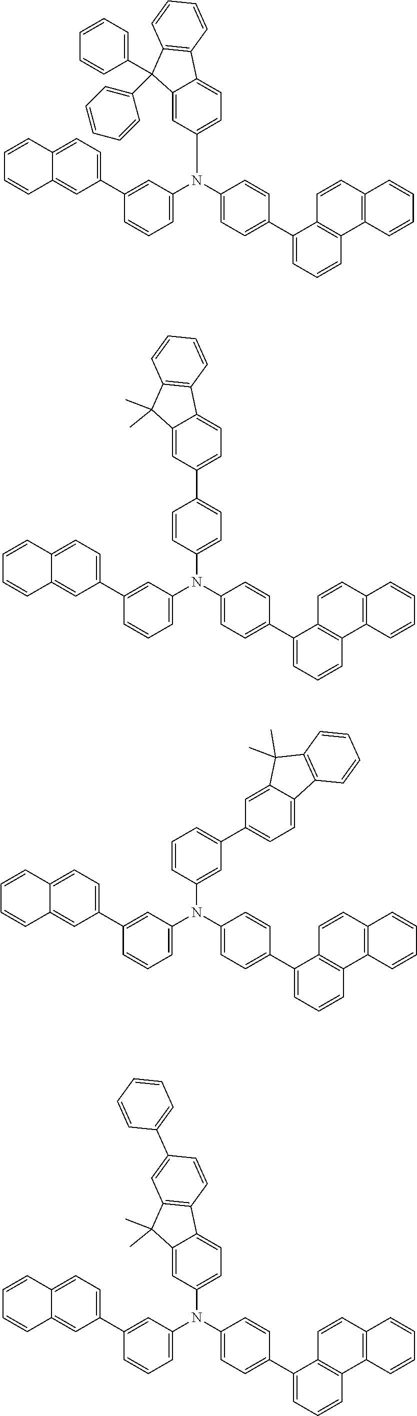 [SMILES: c1ccc(C2(c3ccccc3)c3ccccc3-c3ccc(N(c4ccc(-c5cccc6c5ccc5ccccc56)cc4)c4cccc(-c5ccc6ccccc6c5)c4)cc32)cc1 ; CC1(C)c2ccccc2-c2ccc(-c3ccc(N(c4ccc(-c5cccc6c5ccc5ccccc56)cc4)c4cccc(-c5ccc6ccccc6c5)c4)cc3)cc21 ; CC1(C)c2ccccc2-c2ccc(-c3cccc(N(c4ccc(-c5cccc6c5ccc5ccccc56)cc4)c4cccc(-c5ccc6ccccc6c5)c4)c3)cc21 ; CC1(C)c2cc(-c3ccccc3)ccc2-c2ccc(N(c3ccc(-c4cccc5c4ccc4ccccc45)cc3)c3cccc(-c4ccc5ccccc5c4)c3)cc21]

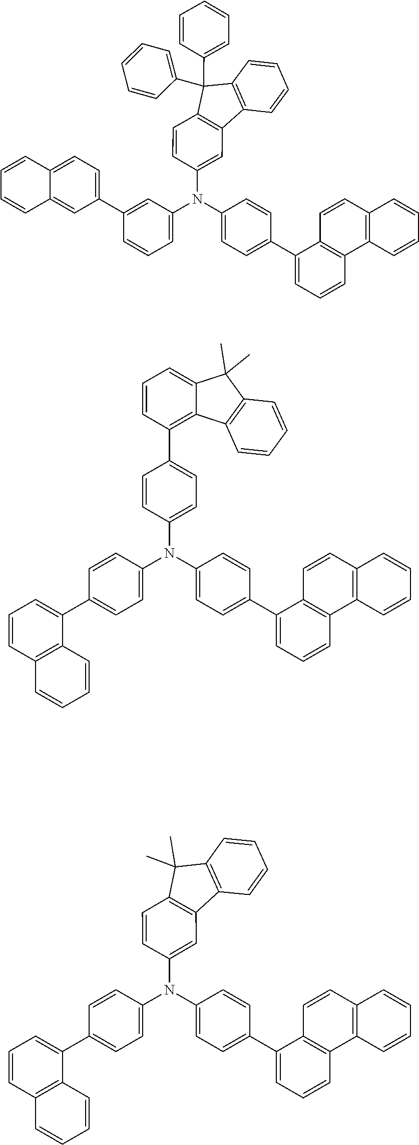 [SMILES: c1ccc(C2(c3ccccc3)c3ccccc3-c3cc(N(c4ccc(-c5cccc6c5ccc5ccccc56)cc4)c4cccc(-c5ccc6ccccc6c5)c4)ccc32)cc1 ; CC1(C)c2ccccc2-c2c(-c3ccc(N(c4ccc(-c5cccc6ccccc56)cc4)c4ccc(-c5cccc6c5ccc5ccccc56)cc4)cc3)cccc21 ; CC1(C)c2ccccc2-c2cc(N(c3ccc(-c4cccc5ccccc45)cc3)c3ccc(-c4cccc5c4ccc4ccccc45)cc3)ccc21]

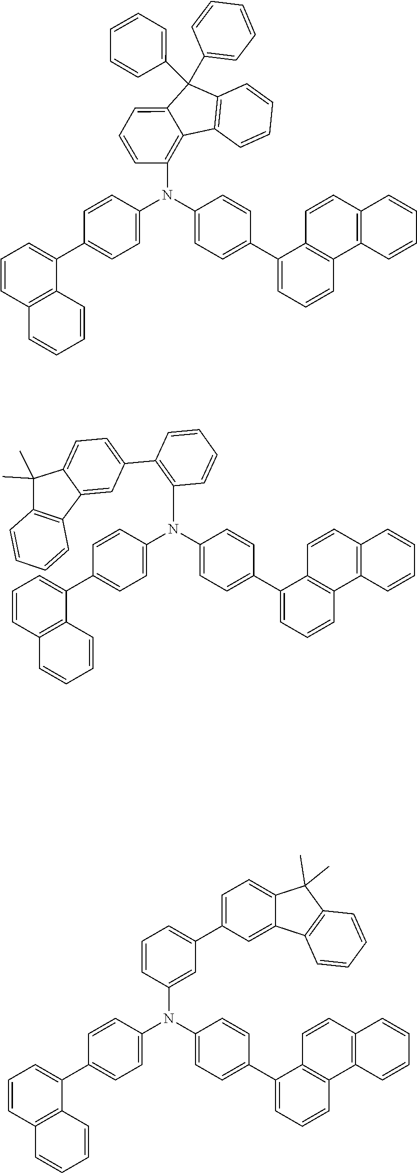 [SMILES: c1ccc(C2(c3ccccc3)c3ccccc3-c3c(N(c4ccc(-c5cccc6ccccc56)cc4)c4ccc(-c5cccc6c5ccc5ccccc56)cc4)cccc32)cc1 ; CC1(C)c2ccccc2-c2cc(-c3ccccc3N(c3ccc(-c4cccc5ccccc45)cc3)c3ccc(-c4cccc5c4ccc4ccccc45)cc3)ccc21 ; CC1(C)c2ccccc2-c2cc(-c3cccc(N(c4ccc(-c5cccc6ccccc56)cc4)c4ccc(-c5cccc6c5ccc5ccccc56)cc4)c3)ccc21]

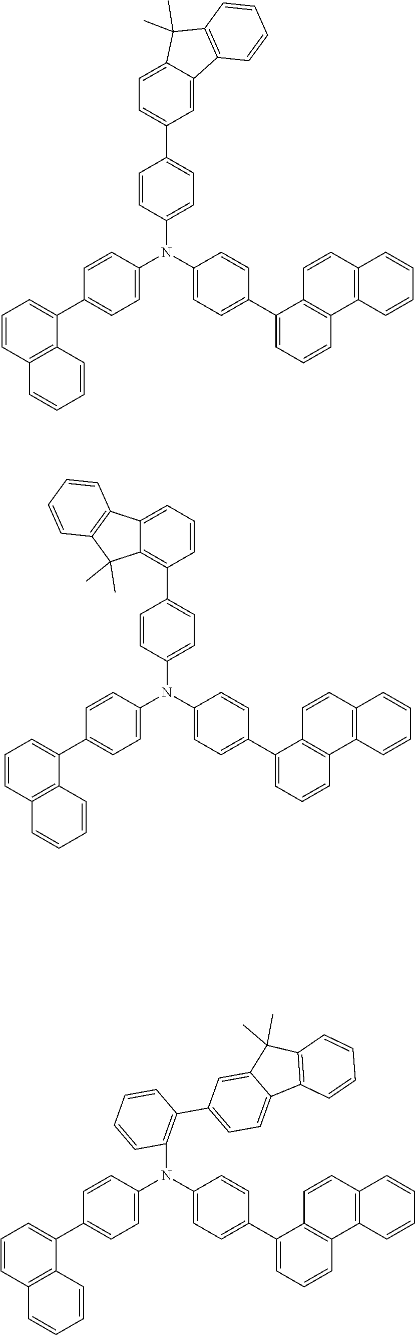 [SMILES: CC1(C)c2ccccc2-c2cc(-c3ccc(N(c4ccc(-c5cccc6ccccc56)cc4)c4ccc(-c5cccc6c5ccc5ccccc56)cc4)cc3)ccc21 ; CC1(C)c2ccccc2-c2cccc(-c3ccc(N(c4ccc(-c5cccc6ccccc56)cc4)c4ccc(-c5cccc6c5ccc5ccccc56)cc4)cc3)c21 ; CC1(C)c2ccccc2-c2ccc(-c3ccccc3N(c3ccc(-c4cccc5ccccc45)cc3)c3ccc(-c4cccc5c4ccc4ccccc45)cc3)cc21]

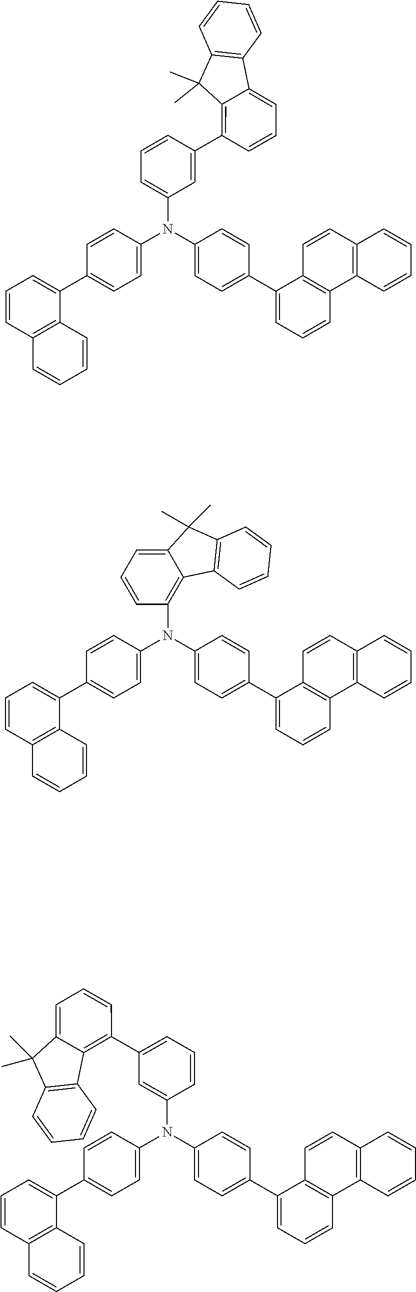 [SMILES: CC1(C)c2ccccc2-c2cccc(-c3cccc(N(c4ccc(-c5cccc6ccccc56)cc4)c4ccc(-c5cccc6c5ccc5ccccc56)cc4)c3)c21 ; CC1(C)c2ccccc2-c2c(N(c3ccc(-c4cccc5ccccc45)cc3)c3ccc(-c4cccc5c4ccc4ccccc45)cc3)cccc21 ; CC1(C)c2ccccc2-c2c(-c3cccc(N(c4ccc(-c5cccc6ccccc56)cc4)c4ccc(-c5cccc6c5ccc5ccccc56)cc4)c3)cccc21]

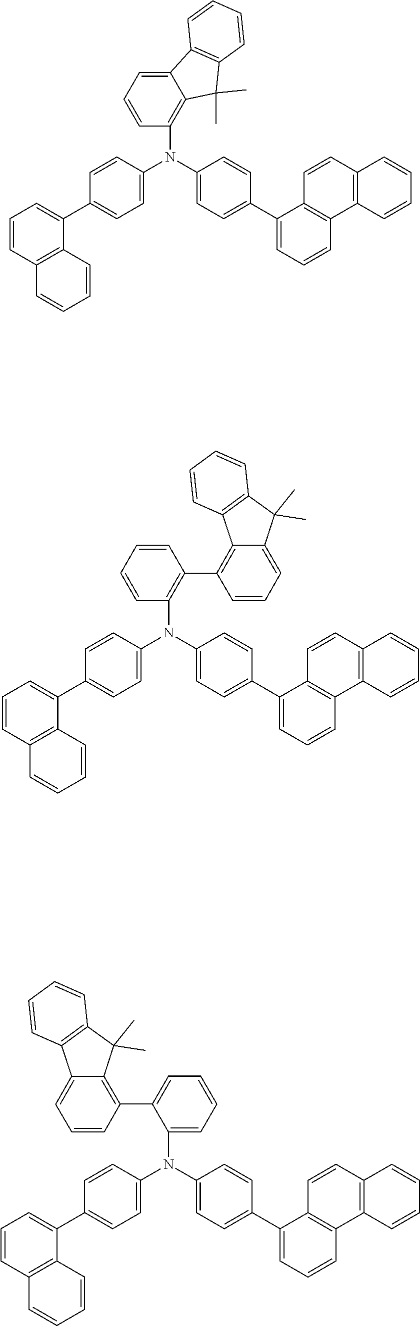 [SMILES: CC1(C)c2ccccc2-c2cccc(N(c3ccc(-c4cccc5ccccc45)cc3)c3ccc(-c4cccc5c4ccc4ccccc45)cc3)c21 ; CC1(C)c2ccccc2-c2c(-c3ccccc3N(c3ccc(-c4cccc5ccccc45)cc3)c3ccc(-c4cccc5c4ccc4ccccc45)cc3)cccc21 ; CC1(C)c2ccccc2-c2cccc(-c3ccccc3N(c3ccc(-c4cccc5ccccc45)cc3)c3ccc(-c4cccc5c4ccc4ccccc45)cc3)c21]

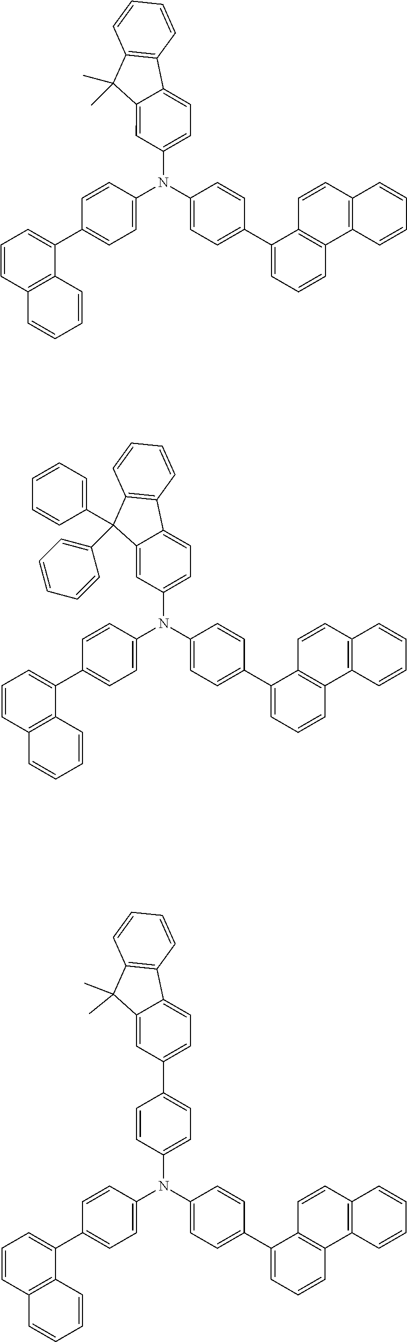 [SMILES: CC1(C)c2ccccc2-c2ccc(N(c3ccc(-c4cccc5ccccc45)cc3)c3ccc(-c4cccc5c4ccc4ccccc45)cc3)cc21 ; c1ccc(C2(c3ccccc3)c3ccccc3-c3ccc(N(c4ccc(-c5cccc6ccccc56)cc4)c4ccc(-c5cccc6c5ccc5ccccc56)cc4)cc32)cc1 ; CC1(C)c2ccccc2-c2ccc(-c3ccc(N(c4ccc(-c5cccc6ccccc56)cc4)c4ccc(-c5cccc6c5ccc5ccccc56)cc4)cc3)cc21]

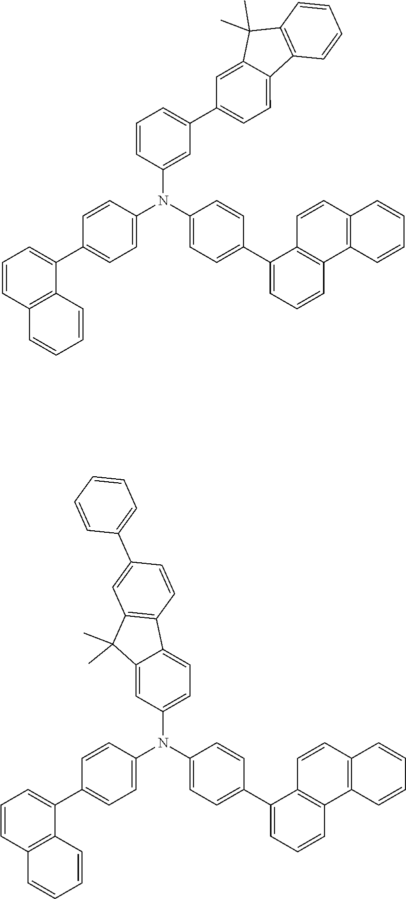 [SMILES: CC1(C)c2ccccc2-c2ccc(-c3cccc(N(c4ccc(-c5cccc6ccccc56)cc4)c4ccc(-c5cccc6c5ccc5ccccc56)cc4)c3)cc21 ; CC1(C)c2cc(-c3ccccc3)ccc2-c2ccc(N(c3ccc(-c4cccc5ccccc45)cc3)c3ccc(-c4cccc5c4ccc4ccccc45)cc3)cc21]

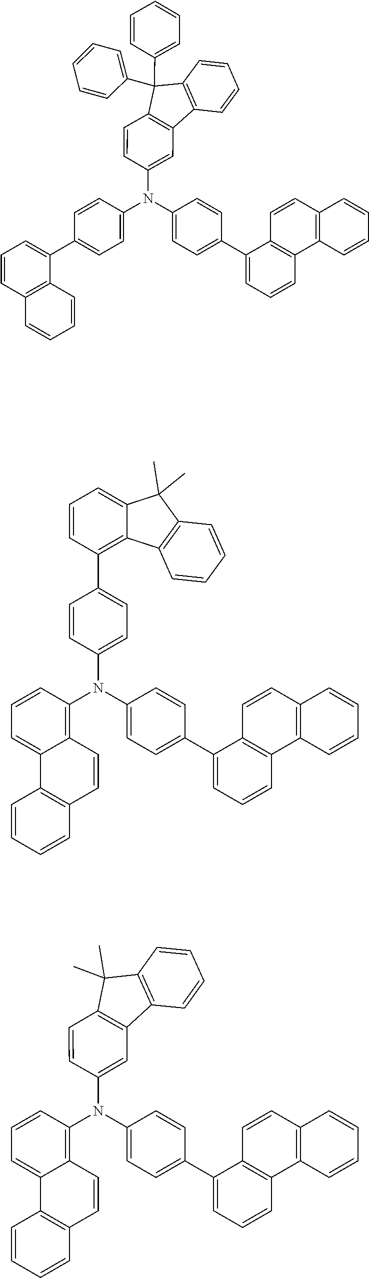 [SMILES: c1ccc(C2(c3ccccc3)c3ccccc3-c3cc(N(c4ccc(-c5cccc6ccccc56)cc4)c4ccc(-c5cccc6c5ccc5ccccc56)cc4)ccc32)cc1 ; CC1(C)c2ccccc2-c2c(-c3ccc(N(c4ccc(-c5cccc6c5ccc5ccccc56)cc4)c4cccc5c4ccc4ccccc45)cc3)cccc21 ; CC1(C)c2ccccc2-c2cc(N(c3ccc(-c4cccc5c4ccc4ccccc45)cc3)c3cccc4c3ccc3ccccc34)ccc21]

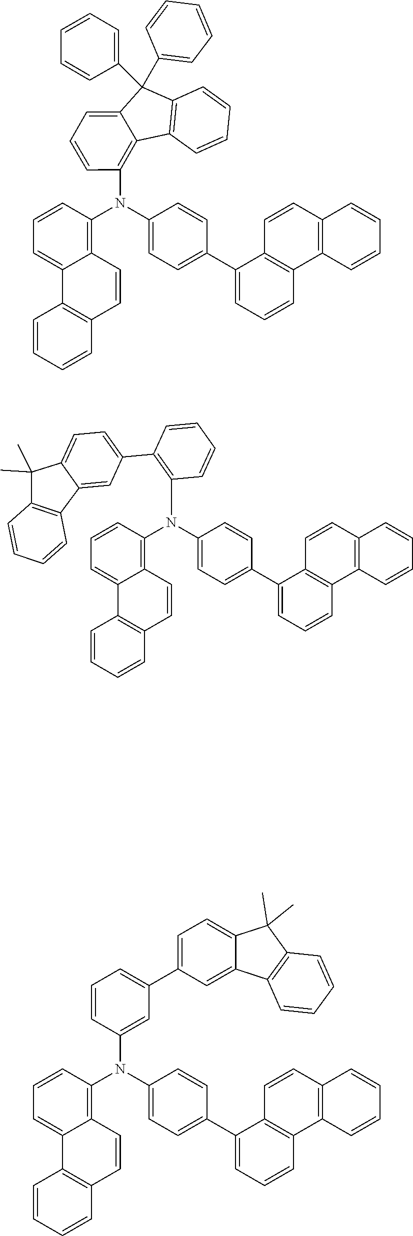 [SMILES: c1ccc(C2(c3ccccc3)c3ccccc3-c3c(N(c4ccc(-c5cccc6c5ccc5ccccc56)cc4)c4cccc5c4ccc4ccccc45)cccc32)cc1 ; CC1(C)c2ccccc2-c2cc(-c3ccccc3N(c3ccc(-c4cccc5c4ccc4ccccc45)cc3)c3cccc4c3ccc3ccccc34)ccc21 ; CC1(C)c2ccccc2-c2cc(-c3cccc(N(c4ccc(-c5cccc6c5ccc5ccccc56)cc4)c4cccc5c4ccc4ccccc45)c3)ccc21]

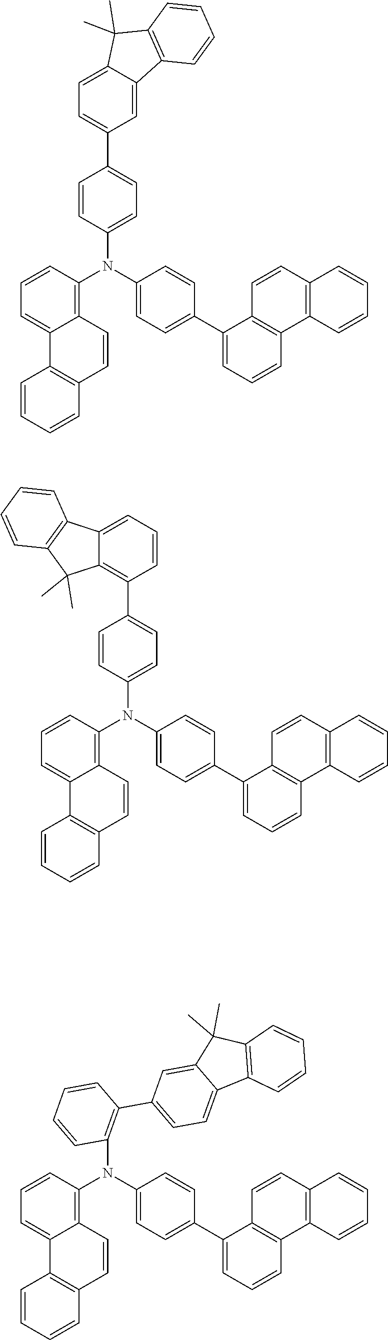 [SMILES: CC1(C)c2ccccc2-c2cc(-c3ccc(N(c4ccc(-c5cccc6c5ccc5ccccc56)cc4)c4cccc5c4ccc4ccccc45)cc3)ccc21 ; CC1(C)c2ccccc2-c2cccc(-c3ccc(N(c4ccc(-c5cccc6c5ccc5ccccc56)cc4)c4cccc5c4ccc4ccccc45)cc3)c21 ; CC1(C)c2ccccc2-c2ccc(-c3ccccc3N(c3ccc(-c4cccc5c4ccc4ccccc45)cc3)c3cccc4c3ccc3ccccc34)cc21]

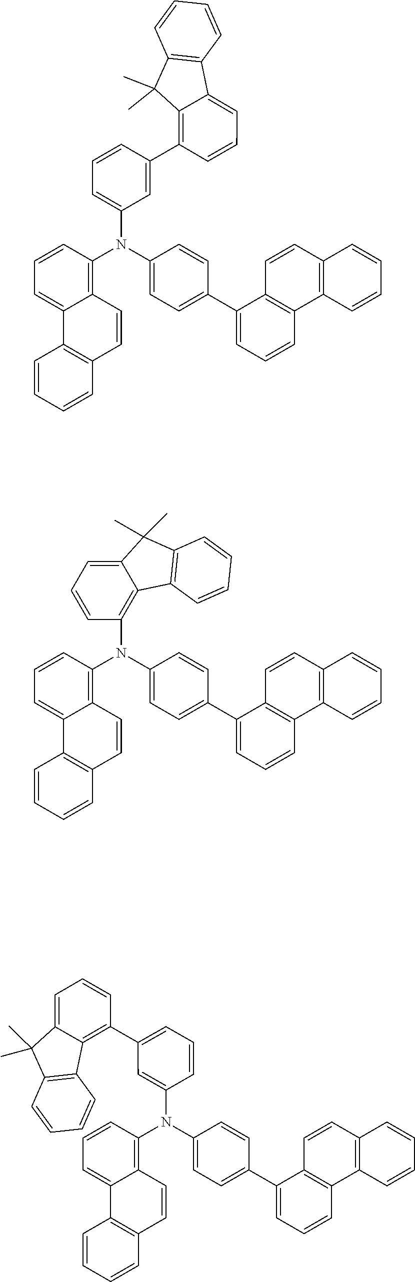 [SMILES: CC1(C)c2ccccc2-c2cccc(-c3cccc(N(c4ccc(-c5cccc6c5ccc5ccccc56)cc4)c4cccc5c4ccc4ccccc45)c3)c21 ; CC1(C)c2ccccc2-c2c(N(c3ccc(-c4cccc5c4ccc4ccccc45)cc3)c3cccc4c3ccc3ccccc34)cccc21 ; CC1(C)c2ccccc2-c2c(-c3cccc(N(c4ccc(-c5cccc6c5ccc5ccccc56)cc4)c4cccc5c4ccc4ccccc45)c3)cccc21]

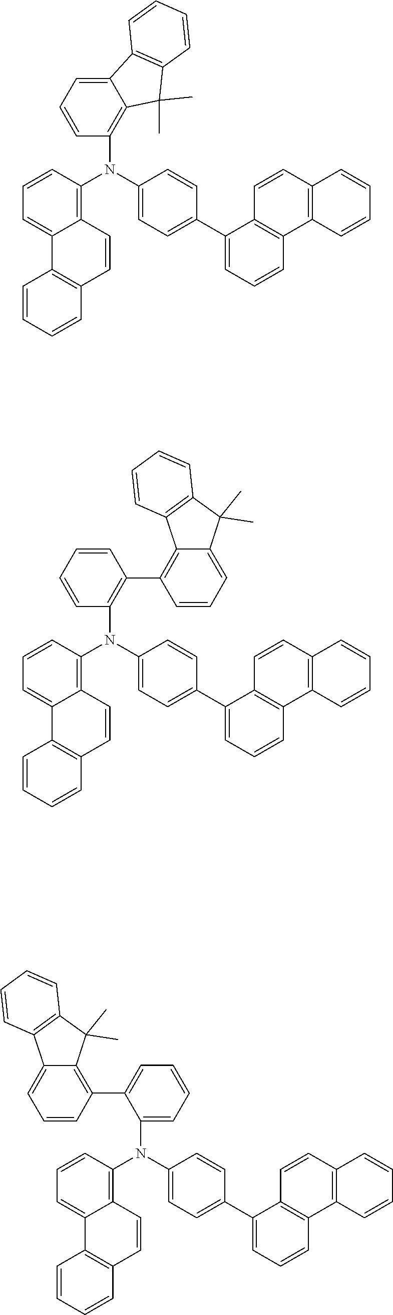 [SMILES: CC1(C)c2ccccc2-c2cccc(N(c3ccc(-c4cccc5c4ccc4ccccc45)cc3)c3cccc4c3ccc3ccccc34)c21 ; CC1(C)c2ccccc2-c2c(-c3ccccc3N(c3ccc(-c4cccc5c4ccc4ccccc45)cc3)c3cccc4c3ccc3ccccc34)cccc21 ; CC1(C)c2ccccc2-c2cccc(-c3ccccc3N(c3ccc(-c4cccc5c4ccc4ccccc45)cc3)c3cccc4c3ccc3ccccc34)c21]

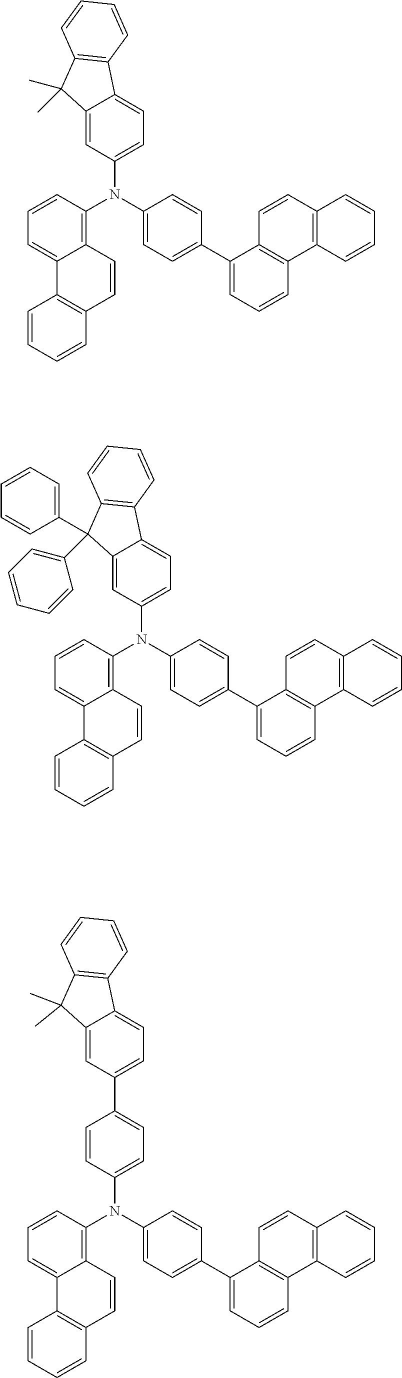 [SMILES: CC1(C)c2ccccc2-c2ccc(N(c3ccc(-c4cccc5c4ccc4ccccc45)cc3)c3cccc4c3ccc3ccccc34)cc21 ; c1ccc(C2(c3ccccc3)c3ccccc3-c3ccc(N(c4ccc(-c5cccc6c5ccc5ccccc56)cc4)c4cccc5c4ccc4ccccc45)cc32)cc1 ; CC1(C)c2ccccc2-c2ccc(-c3ccc(N(c4ccc(-c5cccc6c5ccc5ccccc56)cc4)c4cccc5c4ccc4ccccc45)cc3)cc21]

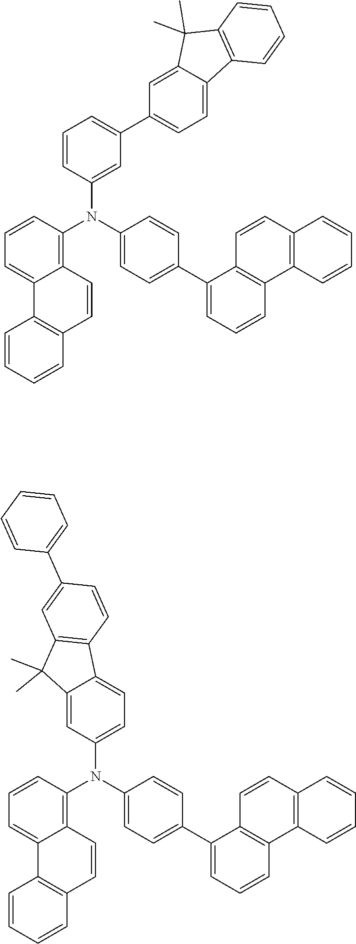 [SMILES: CC1(C)c2ccccc2-c2ccc(-c3cccc(N(c4ccc(-c5cccc6c5ccc5ccccc56)cc4)c4cccc5c4ccc4ccccc45)c3)cc21 ; CC1(C)c2cc(-c3ccccc3)ccc2-c2ccc(N(c3ccc(-c4cccc5c4ccc4ccccc45)cc3)c3cccc4c3ccc3ccccc34)cc21]

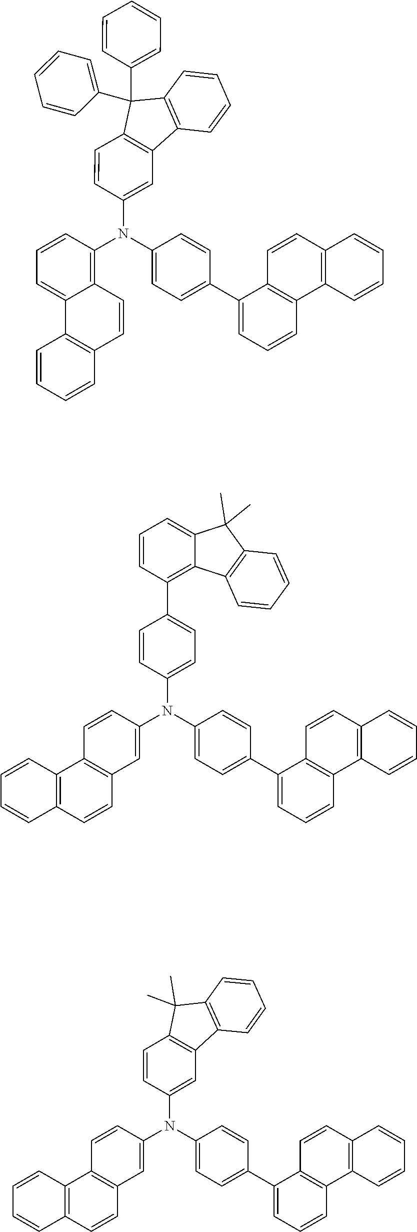 [SMILES: c1ccc(C2(c3ccccc3)c3ccccc3-c3cc(N(c4ccc(-c5cccc6c5ccc5ccccc56)cc4)c4cccc5c4ccc4ccccc45)ccc32)cc1 ; CC1(C)c2ccccc2-c2c(-c3ccc(N(c4ccc(-c5cccc6c5ccc5ccccc56)cc4)c4ccc5c(ccc6ccccc65)c4)cc3)cccc21 ; CC1(C)c2ccccc2-c2cc(N(c3ccc(-c4cccc5c4ccc4ccccc45)cc3)c3ccc4c(ccc5ccccc54)c3)ccc21]

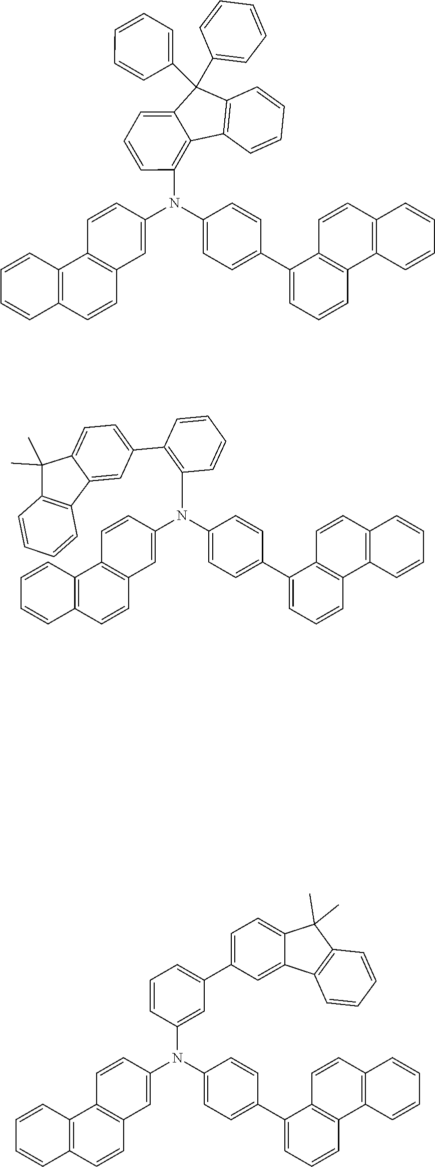 [SMILES: c1ccc(C2(c3ccccc3)c3ccccc3-c3c(N(c4ccc(-c5cccc6c5ccc5ccccc56)cc4)c4ccc5c(ccc6ccccc65)c4)cccc32)cc1 ; CC1(C)c2ccccc2-c2cc(-c3ccccc3N(c3ccc(-c4cccc5c4ccc4ccccc45)cc3)c3ccc4c(ccc5ccccc54)c3)ccc21 ; CC1(C)c2ccccc2-c2cc(-c3cccc(N(c4ccc(-c5cccc6c5ccc5ccccc56)cc4)c4ccc5c(ccc6ccccc65)c4)c3)ccc21]

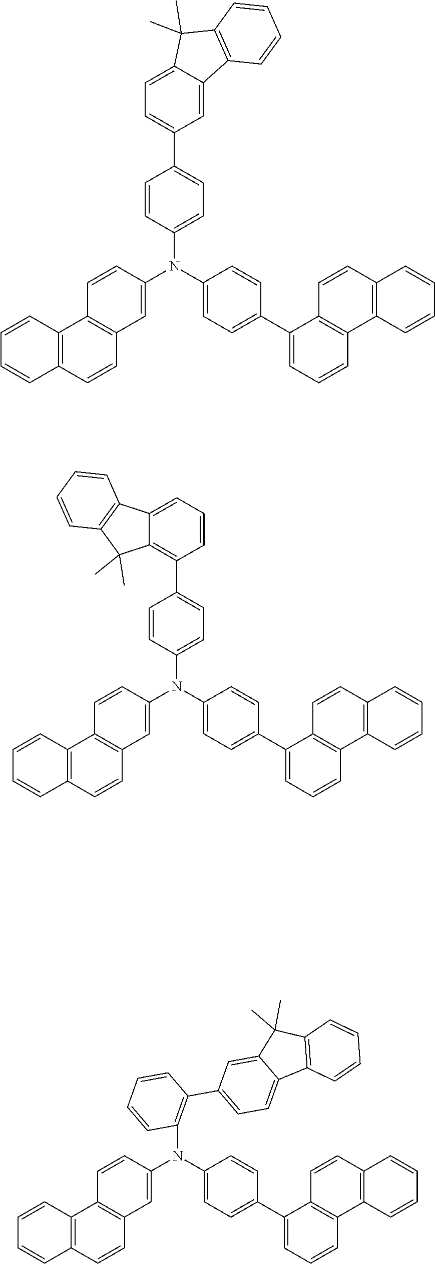 [SMILES: CC1(C)c2ccccc2-c2cc(-c3ccc(N(c4ccc(-c5cccc6c5ccc5ccccc56)cc4)c4ccc5c(ccc6ccccc65)c4)cc3)ccc21 ; CC1(C)c2ccccc2-c2cccc(-c3ccc(N(c4ccc(-c5cccc6c5ccc5ccccc56)cc4)c4ccc5c(ccc6ccccc65)c4)cc3)c21 ; CC1(C)c2ccccc2-c2ccc(-c3ccccc3N(c3ccc(-c4cccc5c4ccc4ccccc45)cc3)c3ccc4c(ccc5ccccc54)c3)cc21]

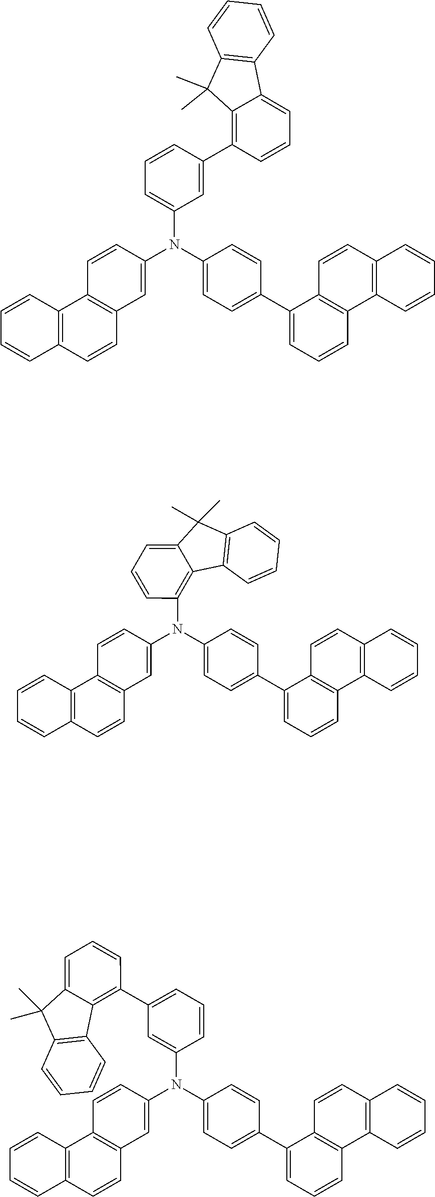 [SMILES: CC1(C)c2ccccc2-c2cccc(-c3cccc(N(c4ccc(-c5cccc6c5ccc5ccccc56)cc4)c4ccc5c(ccc6ccccc65)c4)c3)c21 ; CC1(C)c2ccccc2-c2c(N(c3ccc(-c4cccc5c4ccc4ccccc45)cc3)c3ccc4c(ccc5ccccc54)c3)cccc21 ; CC1(C)c2ccccc2-c2c(-c3cccc(N(c4ccc(-c5cccc6c5ccc5ccccc56)cc4)c4ccc5c(ccc6ccccc65)c4)c3)cccc21]

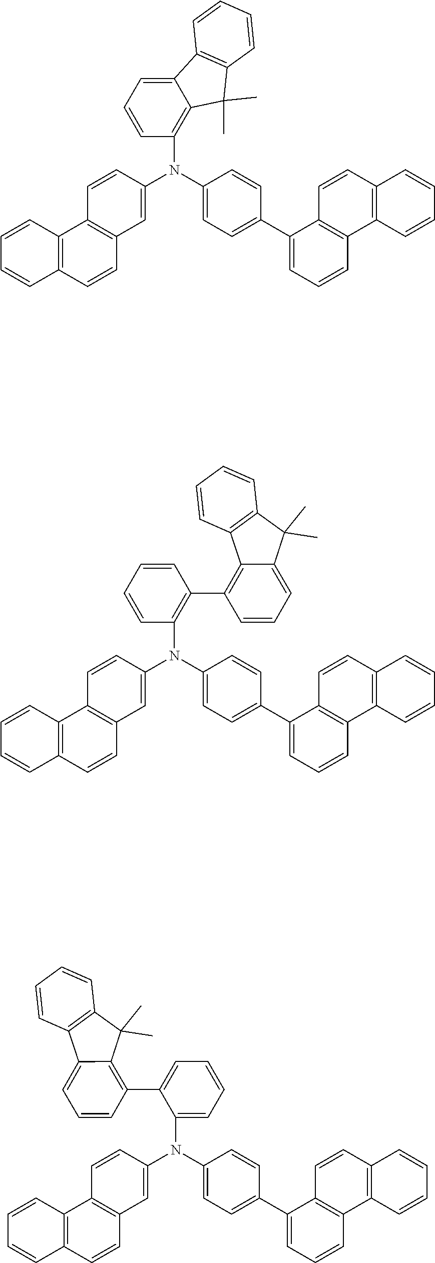 [SMILES: CC1(C)c2ccccc2-c2cccc(N(c3ccc(-c4cccc5c4ccc4ccccc45)cc3)c3ccc4c(ccc5ccccc54)c3)c21 ; CC1(C)c2ccccc2-c2c(-c3ccccc3N(c3ccc(-c4cccc5c4ccc4ccccc45)cc3)c3ccc4c(ccc5ccccc54)c3)cccc21 ; CC1(C)c2ccccc2-c2cccc(-c3ccccc3N(c3ccc(-c4cccc5c4ccc4ccccc45)cc3)c3ccc4c(ccc5ccccc54)c3)c21]

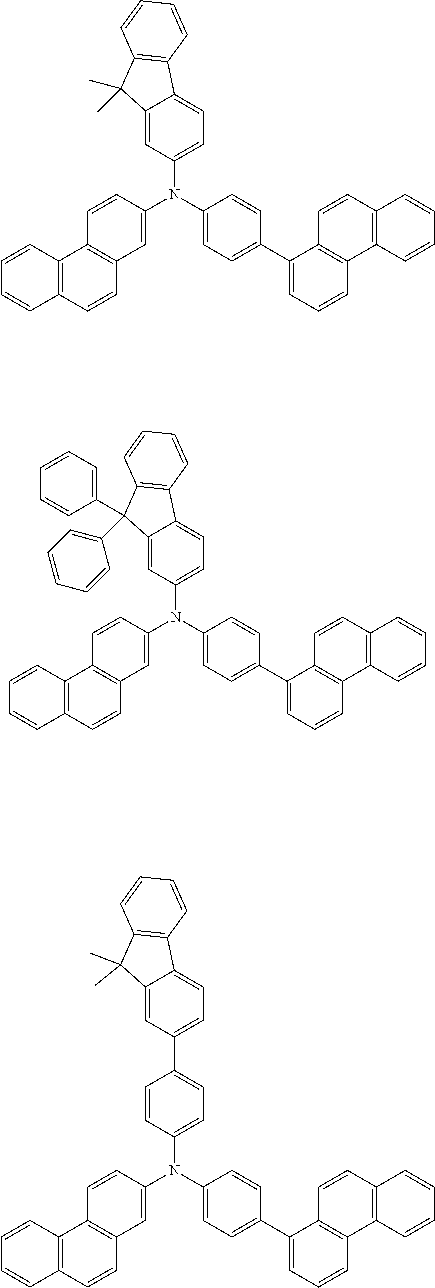 [SMILES: CC1(C)c2ccccc2-c2ccc(N(c3ccc(-c4cccc5c4ccc4ccccc45)cc3)c3ccc4c(ccc5ccccc54)c3)cc21 ; c1ccc(C2(c3ccccc3)c3ccccc3-c3ccc(N(c4ccc(-c5cccc6c5ccc5ccccc56)cc4)c4ccc5c(ccc6ccccc65)c4)cc32)cc1 ; CC1(C)c2ccccc2-c2ccc(-c3ccc(N(c4ccc(-c5cccc6c5ccc5ccccc56)cc4)c4ccc5c(ccc6ccccc65)c4)cc3)cc21]

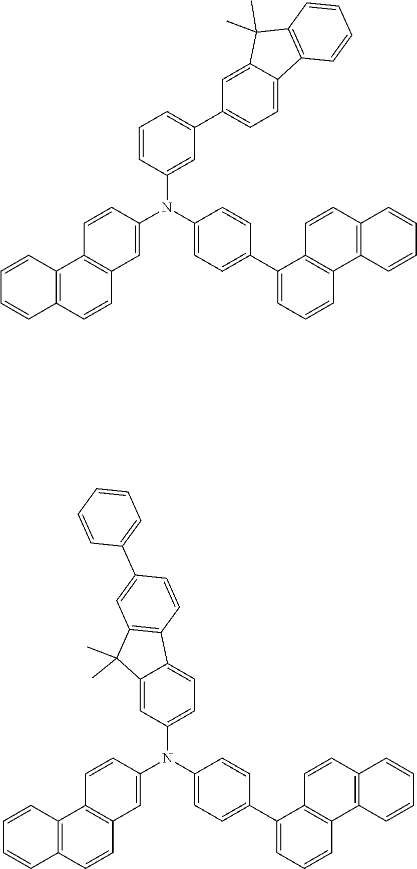 [SMILES: CC1(C)c2ccccc2-c2ccc(-c3cccc(N(c4ccc(-c5cccc6c5ccc5ccccc56)cc4)c4ccc5c(ccc6ccccc65)c4)c3)cc21 ; CC1(C)c2cc(-c3ccccc3)ccc2-c2ccc(N(c3ccc(-c4cccc5c4ccc4ccccc45)cc3)c3ccc4c(ccc5ccccc54)c3)cc21]

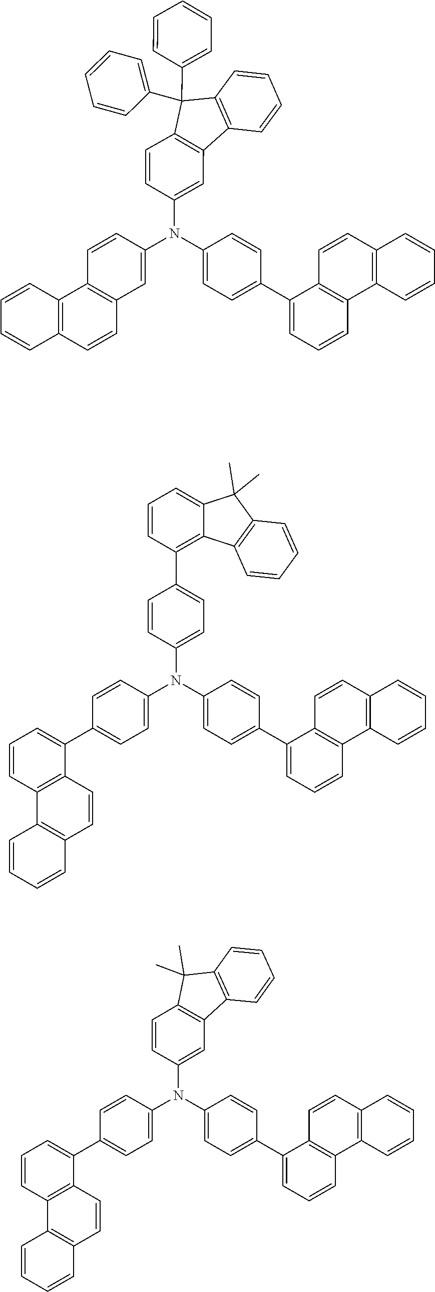 [SMILES: c1ccc(C2(c3ccccc3)c3ccccc3-c3cc(N(c4ccc(-c5cccc6c5ccc5ccccc56)cc4)c4ccc5c(ccc6ccccc65)c4)ccc32)cc1 ; CC1(C)c2ccccc2-c2c(-c3ccc(N(c4ccc(-c5cccc6c5ccc5ccccc56)cc4)c4ccc(-c5cccc6c5ccc5ccccc56)cc4)cc3)cccc21 ; CC1(C)c2ccccc2-c2cc(N(c3ccc(-c4cccc5c4ccc4ccccc45)cc3)c3ccc(-c4cccc5c4ccc4ccccc45)cc3)ccc21]

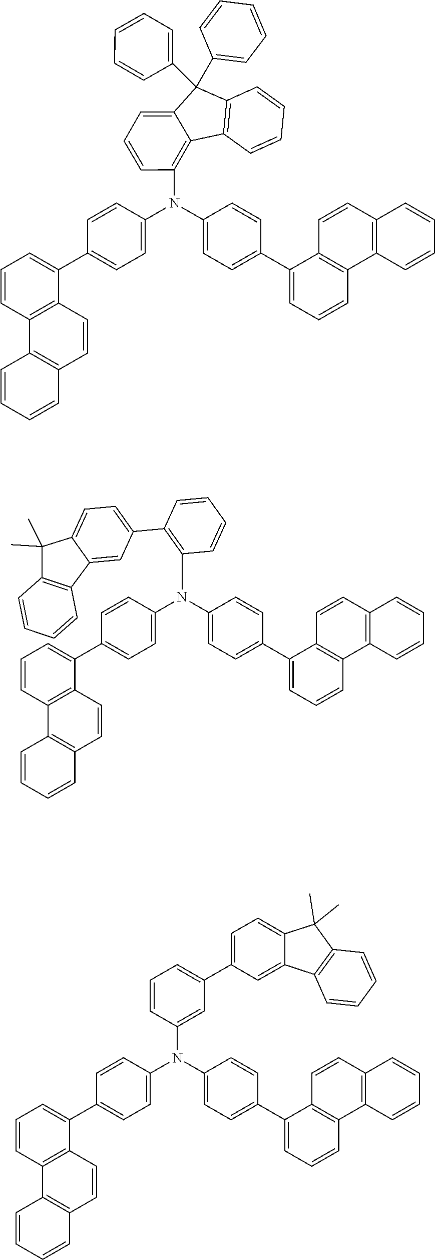 [SMILES: c1ccc(C2(c3ccccc3)c3ccccc3-c3c(N(c4ccc(-c5cccc6c5ccc5ccccc56)cc4)c4ccc(-c5cccc6c5ccc5ccccc56)cc4)cccc32)cc1 ; CC1(C)c2ccccc2-c2cc(-c3ccccc3N(c3ccc(-c4cccc5c4ccc4ccccc45)cc3)c3ccc(-c4cccc5c4ccc4ccccc45)cc3)ccc21 ; CC1(C)c2ccccc2-c2cc(-c3cccc(N(c4ccc(-c5cccc6c5ccc5ccccc56)cc4)c4ccc(-c5cccc6c5ccc5ccccc56)cc4)c3)ccc21]

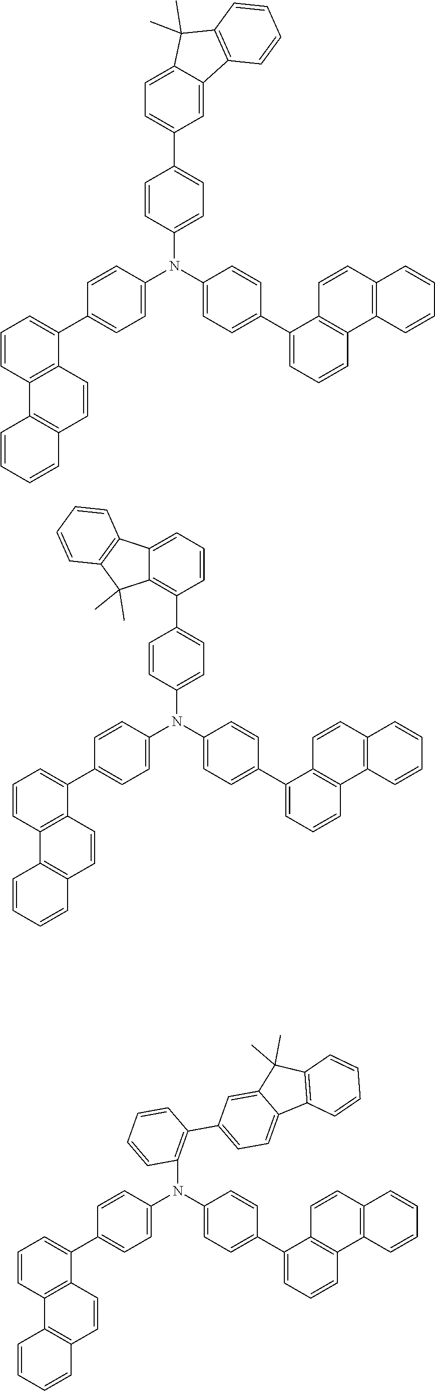

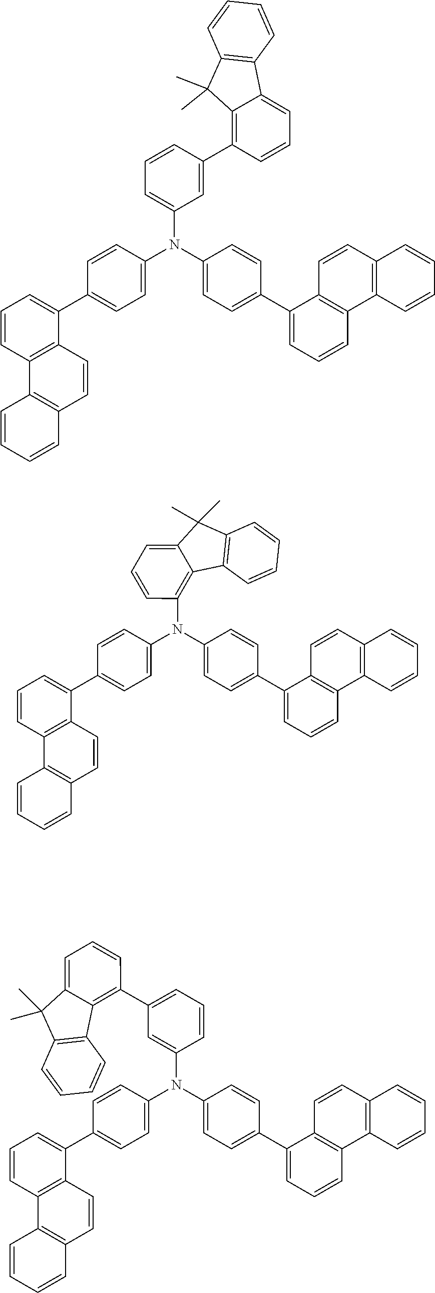 [SMILES: CC1(C)c2ccccc2-c2cccc(-c3cccc(N(c4ccc(-c5cccc6c5ccc5ccccc56)cc4)c4ccc(-c5cccc6c5ccc5ccccc56)cc4)c3)c21 ; CC1(C)c2ccccc2-c2c(N(c3ccc(-c4cccc5c4ccc4ccccc45)cc3)c3ccc(-c4cccc5c4ccc4ccccc45)cc3)cccc21 ; CC1(C)c2ccccc2-c2c(-c3cccc(N(c4ccc(-c5cccc6c5ccc5ccccc56)cc4)c4ccc(-c5cccc6c5ccc5ccccc56)cc4)c3)cccc21]

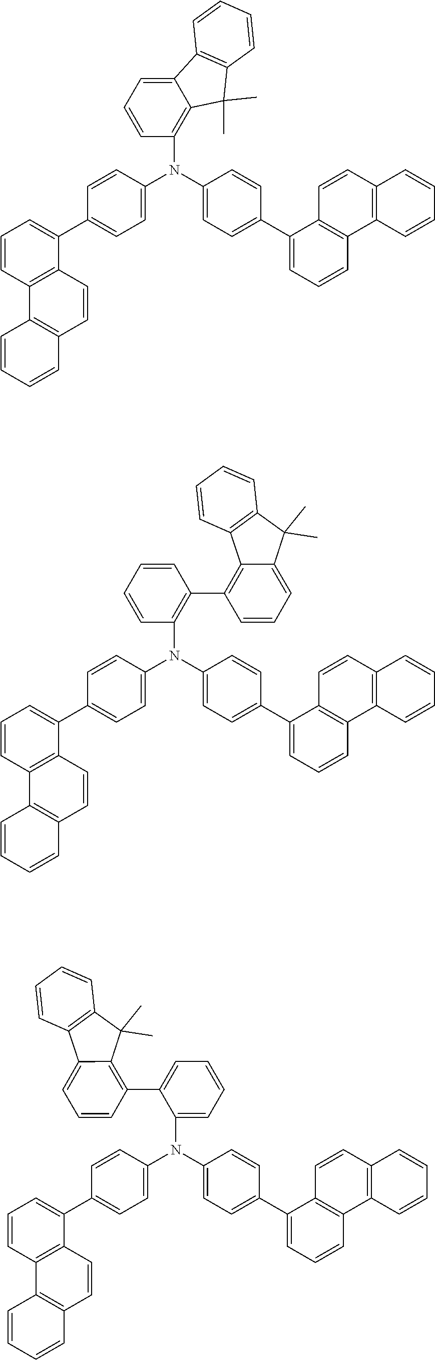 [SMILES: CC1(C)c2ccccc2-c2cccc(N(c3ccc(-c4cccc5c4ccc4ccccc45)cc3)c3ccc(-c4cccc5c4ccc4ccccc45)cc3)c21 ; CC1(C)c2ccccc2-c2c(-c3ccccc3N(c3ccc(-c4cccc5c4ccc4ccccc45)cc3)c3ccc(-c4cccc5c4ccc4ccccc45)cc3)cccc21 ; CC1(C)c2ccccc2-c2cccc(-c3ccccc3N(c3ccc(-c4cccc5c4ccc4ccccc45)cc3)c3ccc(-c4cccc5c4ccc4ccccc45)cc3)c21]

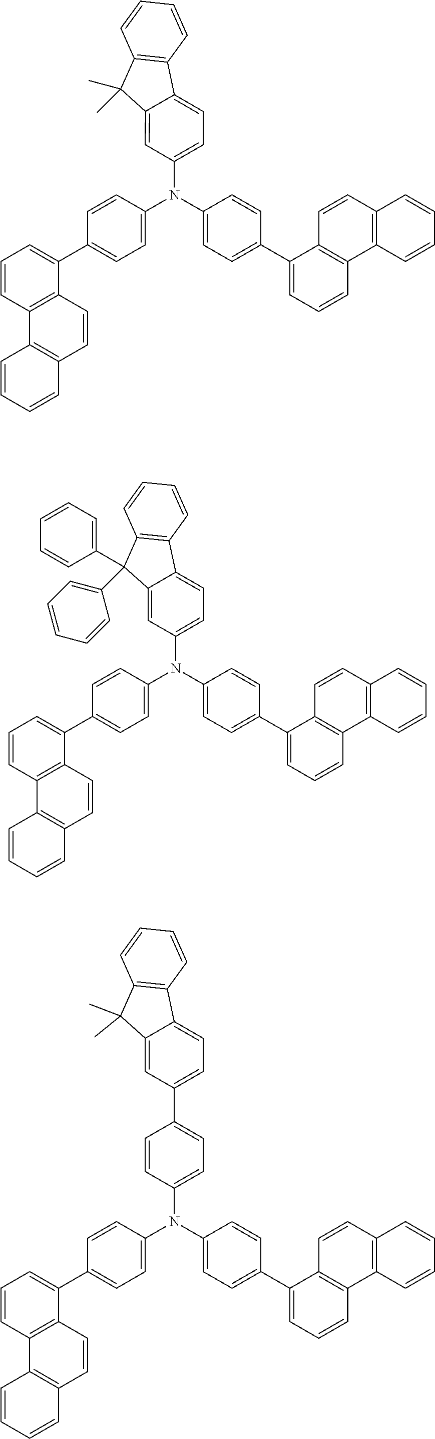 [SMILES: CC1(C)c2ccccc2-c2ccc(N(c3ccc(-c4cccc5c4ccc4ccccc45)cc3)c3ccc(-c4cccc5c4ccc4ccccc45)cc3)cc21 ; c1ccc(C2(c3ccccc3)c3ccccc3-c3ccc(N(c4ccc(-c5cccc6c5ccc5ccccc56)cc4)c4ccc(-c5cccc6c5ccc5ccccc56)cc4)cc32)cc1 ; CC1(C)c2ccccc2-c2ccc(-c3ccc(N(c4ccc(-c5cccc6c5ccc5ccccc56)cc4)c4ccc(-c5cccc6c5ccc5ccccc56)cc4)cc3)cc21]

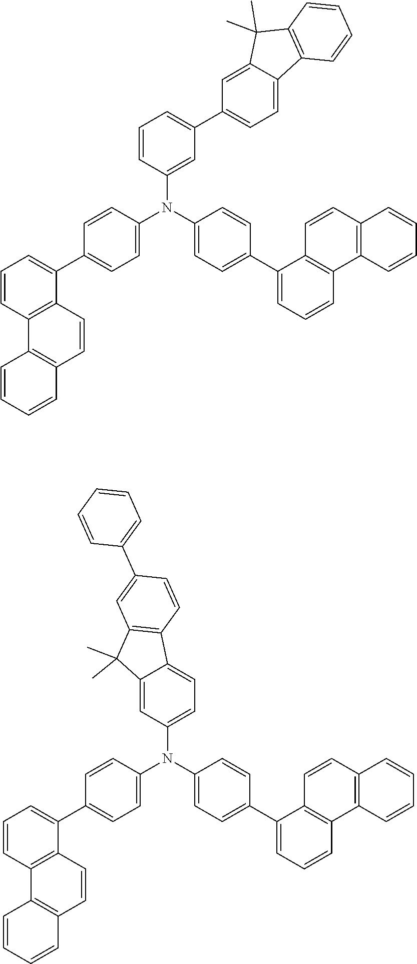 [SMILES: CC1(C)c2ccccc2-c2ccc(-c3cccc(N(c4ccc(-c5cccc6c5ccc5ccccc56)cc4)c4ccc(-c5cccc6c5ccc5ccccc56)cc4)c3)cc21 ; CC1(C)c2cc(-c3ccccc3)ccc2-c2ccc(N(c3ccc(-c4cccc5c4ccc4ccccc45)cc3)c3ccc(-c4cccc5c4ccc4ccccc45)cc3)cc21]

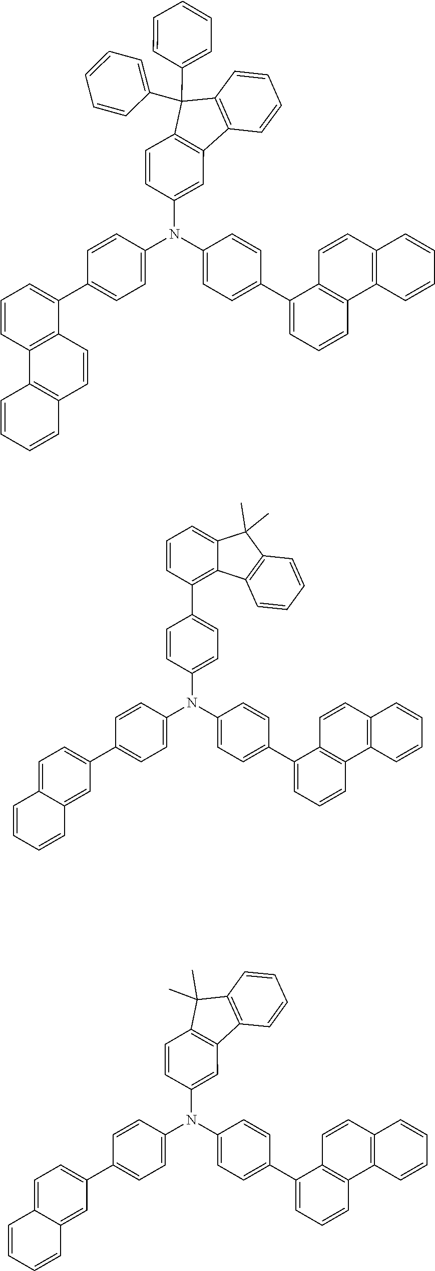 [SMILES: c1ccc(C2(c3ccccc3)c3ccccc3-c3cc(N(c4ccc(-c5cccc6c5ccc5ccccc56)cc4)c4ccc(-c5cccc6c5ccc5ccccc56)cc4)ccc32)cc1 ; CC1(C)c2ccccc2-c2c(-c3ccc(N(c4ccc(-c5ccc6ccccc6c5)cc4)c4ccc(-c5cccc6c5ccc5ccccc56)cc4)cc3)cccc21 ; CC1(C)c2ccccc2-c2cc(N(c3ccc(-c4ccc5ccccc5c4)cc3)c3ccc(-c4cccc5c4ccc4ccccc45)cc3)ccc21]

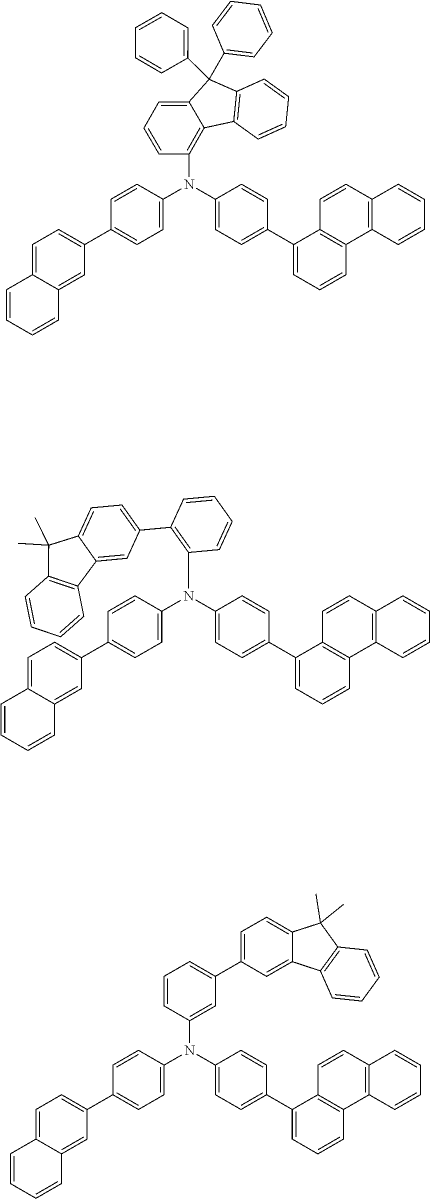 [SMILES: c1ccc(C2(c3ccccc3)c3ccccc3-c3c(N(c4ccc(-c5ccc6ccccc6c5)cc4)c4ccc(-c5cccc6c5ccc5ccccc56)cc4)cccc32)cc1 ; CC1(C)c2ccccc2-c2cc(-c3ccccc3N(c3ccc(-c4ccc5ccccc5c4)cc3)c3ccc(-c4cccc5c4ccc4ccccc45)cc3)ccc21 ; CC1(C)c2ccccc2-c2cc(-c3cccc(N(c4ccc(-c5ccc6ccccc6c5)cc4)c4ccc(-c5cccc6c5ccc5ccccc56)cc4)c3)ccc21]

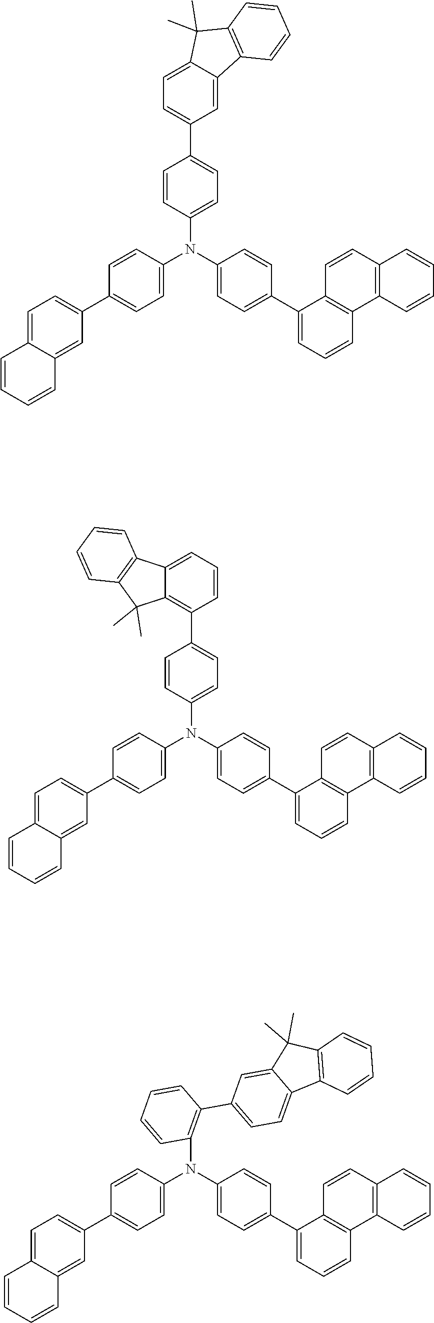 [SMILES: CC1(C)c2ccccc2-c2cc(-c3ccc(N(c4ccc(-c5ccc6ccccc6c5)cc4)c4ccc(-c5cccc6c5ccc5ccccc56)cc4)cc3)ccc21 ; CC1(C)c2ccccc2-c2cccc(-c3ccc(N(c4ccc(-c5ccc6ccccc6c5)cc4)c4ccc(-c5cccc6c5ccc5ccccc56)cc4)cc3)c21 ; CC1(C)c2ccccc2-c2ccc(-c3ccccc3N(c3ccc(-c4ccc5ccccc5c4)cc3)c3ccc(-c4cccc5c4ccc4ccccc45)cc3)cc21]

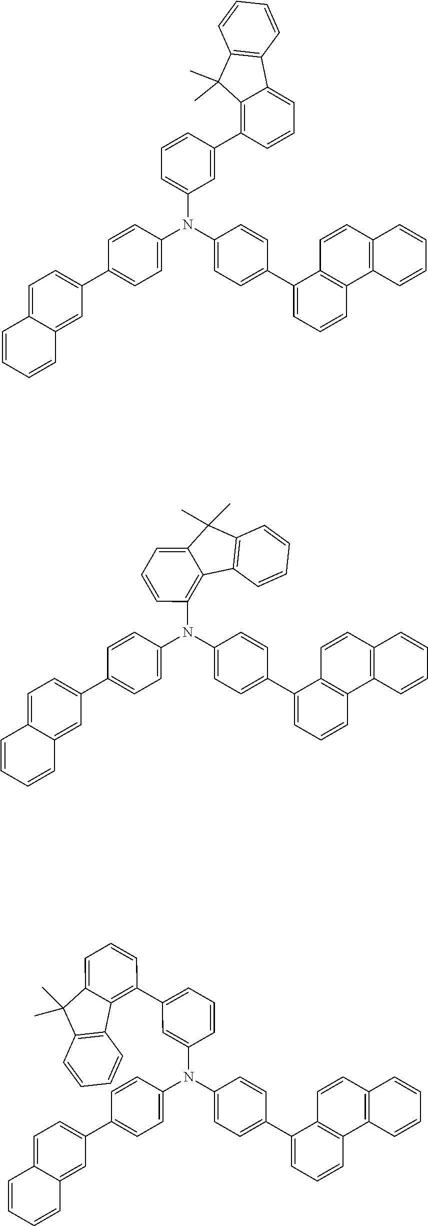 [SMILES: CC1(C)c2ccccc2-c2cccc(-c3cccc(N(c4ccc(-c5ccc6ccccc6c5)cc4)c4ccc(-c5cccc6c5ccc5ccccc56)cc4)c3)c21 ; CC1(C)c2ccccc2-c2c(N(c3ccc(-c4ccc5ccccc5c4)cc3)c3ccc(-c4cccc5c4ccc4ccccc45)cc3)cccc21 ; CC1(C)c2ccccc2-c2c(-c3cccc(N(c4ccc(-c5ccc6ccccc6c5)cc4)c4ccc(-c5cccc6c5ccc5ccccc56)cc4)c3)cccc21]

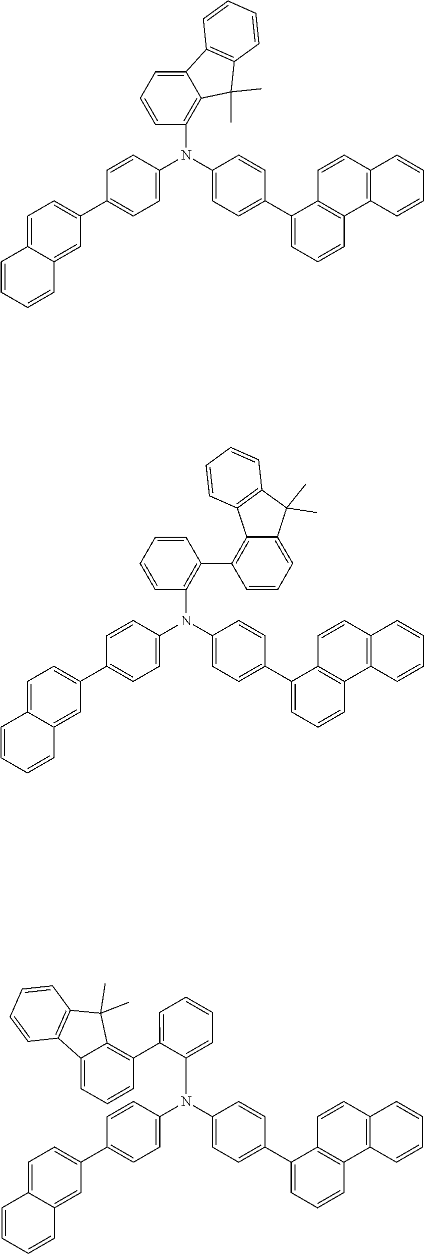 [SMILES: CC1(C)c2ccccc2-c2cccc(N(c3ccc(-c4ccc5ccccc5c4)cc3)c3ccc(-c4cccc5c4ccc4ccccc45)cc3)c21 ; CC1(C)c2ccccc2-c2c(-c3ccccc3N(c3ccc(-c4ccc5ccccc5c4)cc3)c3ccc(-c4cccc5c4ccc4ccccc45)cc3)cccc21 ; CC1(C)c2ccccc2-c2cccc(-c3ccccc3N(c3ccc(-c4ccc5ccccc5c4)cc3)c3ccc(-c4cccc5c4ccc4ccccc45)cc3)c21]

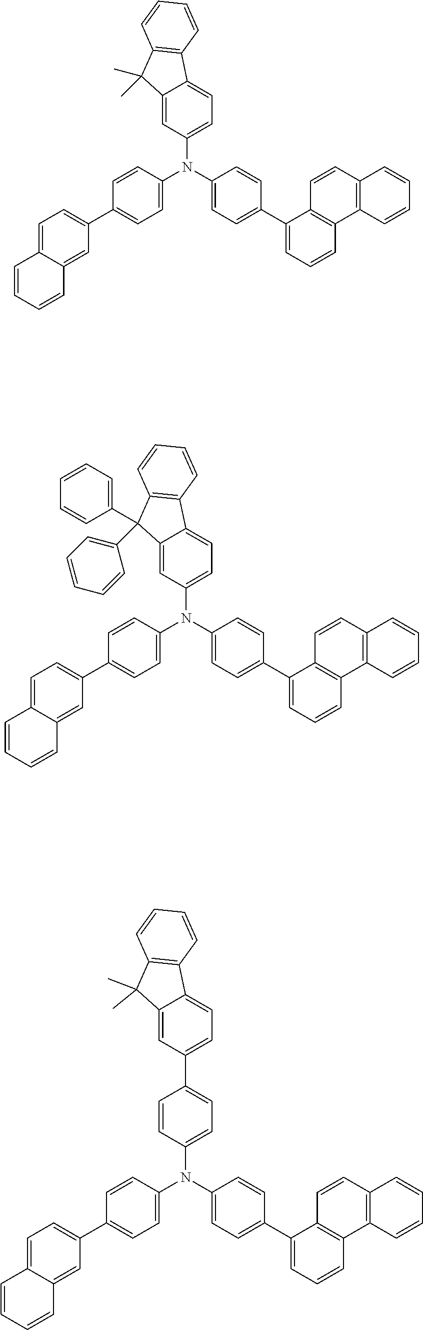 [SMILES: CC1(C)c2ccccc2-c2ccc(N(c3ccc(-c4ccc5ccccc5c4)cc3)c3ccc(-c4cccc5c4ccc4ccccc45)cc3)cc21 ; c1ccc(C2(c3ccccc3)c3ccccc3-c3ccc(N(c4ccc(-c5ccc6ccccc6c5)cc4)c4ccc(-c5cccc6c5ccc5ccccc56)cc4)cc32)cc1 ; CC1(C)c2ccccc2-c2ccc(-c3ccc(N(c4ccc(-c5ccc6ccccc6c5)cc4)c4ccc(-c5cccc6c5ccc5ccccc56)cc4)cc3)cc21]

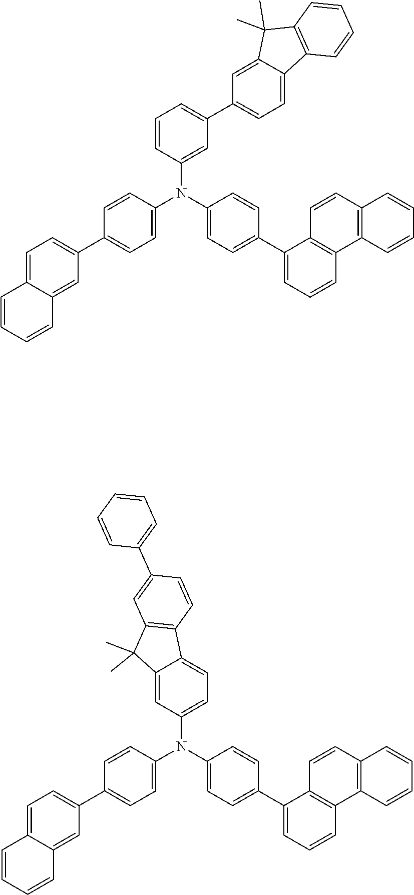 [SMILES: CC1(C)c2ccccc2-c2ccc(-c3cccc(N(c4ccc(-c5ccc6ccccc6c5)cc4)c4ccc(-c5cccc6c5ccc5ccccc56)cc4)c3)cc21 ; CC1(C)c2cc(-c3ccccc3)ccc2-c2ccc(N(c3ccc(-c4ccc5ccccc5c4)cc3)c3ccc(-c4cccc5c4ccc4ccccc45)cc3)cc21]

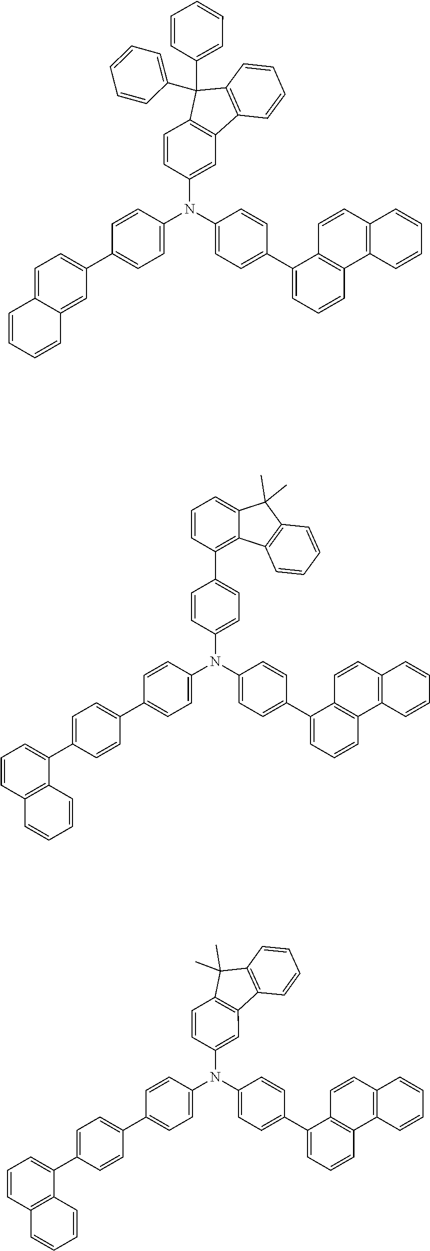 [SMILES: c1ccc(C2(c3ccccc3)c3ccccc3-c3cc(N(c4ccc(-c5ccc6ccccc6c5)cc4)c4ccc(-c5cccc6c5ccc5ccccc56)cc4)ccc32)cc1 ; CC1(C)c2ccccc2-c2c(-c3ccc(N(c4ccc(-c5ccc(-c6cccc7ccccc67)cc5)cc4)c4ccc(-c5cccc6c5ccc5ccccc56)cc4)cc3)cccc21 ; CC1(C)c2ccccc2-c2cc(N(c3ccc(-c4ccc(-c5cccc6ccccc56)cc4)cc3)c3ccc(-c4cccc5c4ccc4ccccc45)cc3)ccc21]

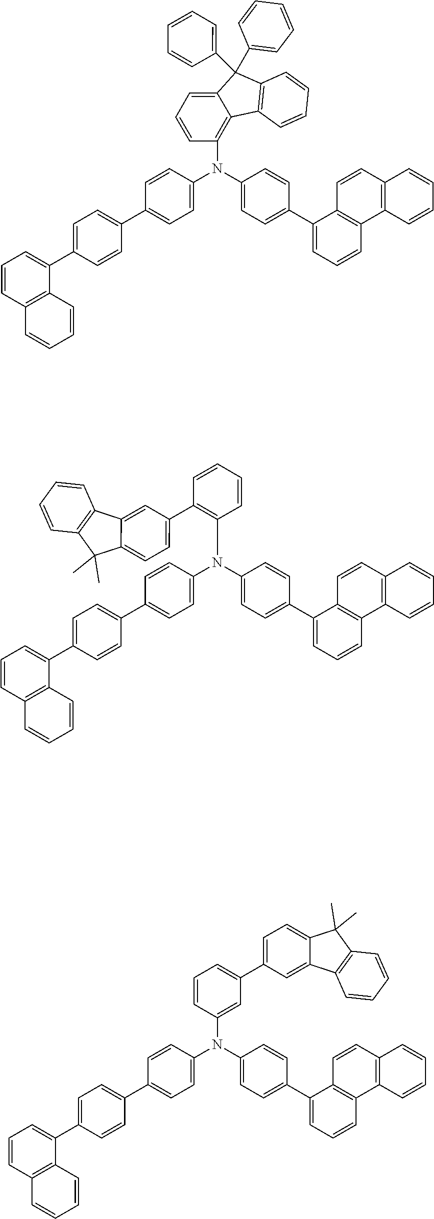 [SMILES: c1ccc(C2(c3ccccc3)c3ccccc3-c3c(N(c4ccc(-c5ccc(-c6cccc7ccccc67)cc5)cc4)c4ccc(-c5cccc6c5ccc5ccccc56)cc4)cccc32)cc1 ; CC1(C)c2ccccc2-c2cc(-c3ccccc3N(c3ccc(-c4ccc(-c5cccc6ccccc56)cc4)cc3)c3ccc(-c4cccc5c4ccc4ccccc45)cc3)ccc21 ; CC1(C)c2ccccc2-c2cc(-c3cccc(N(c4ccc(-c5ccc(-c6cccc7ccccc67)cc5)cc4)c4ccc(-c5cccc6c5ccc5ccccc56)cc4)c3)ccc21]

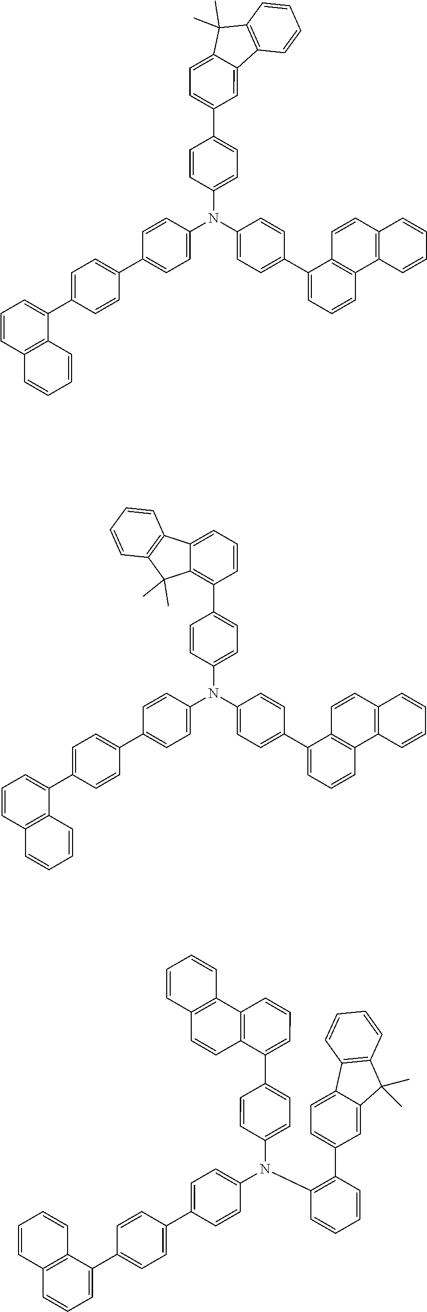 [SMILES: CC1(C)c2ccccc2-c2cc(-c3ccc(N(c4ccc(-c5ccc(-c6cccc7ccccc67)cc5)cc4)c4ccc(-c5cccc6c5ccc5ccccc56)cc4)cc3)ccc21 ; CC1(C)c2ccccc2-c2cccc(-c3ccc(N(c4ccc(-c5ccc(-c6cccc7ccccc67)cc5)cc4)c4ccc(-c5cccc6c5ccc5ccccc56)cc4)cc3)c21 ; CC1(C)c2ccccc2-c2ccc(-c3ccccc3N(c3ccc(-c4ccc(-c5cccc6ccccc56)cc4)cc3)c3ccc(-c4cccc5c4ccc4ccccc45)cc3)cc21]

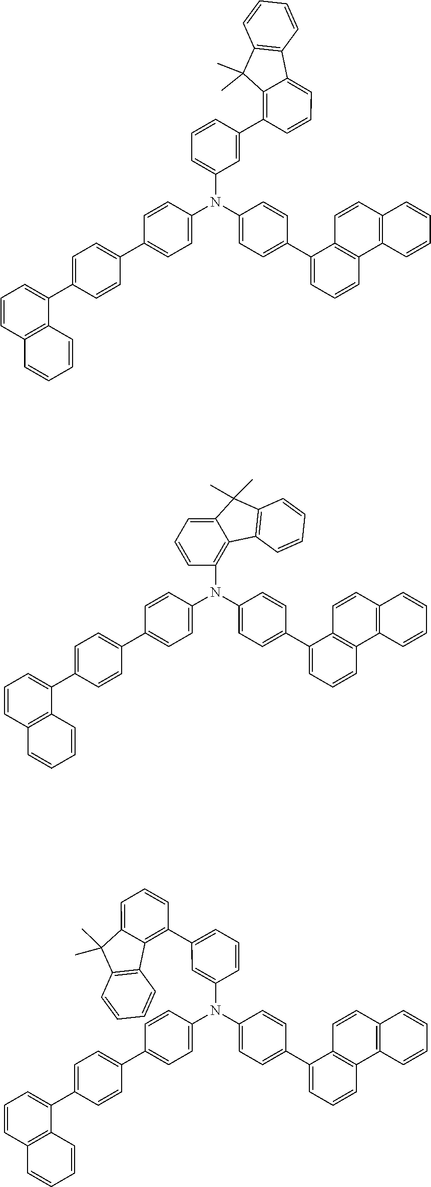 [SMILES: CC1(C)c2ccccc2-c2cccc(-c3cccc(N(c4ccc(-c5ccc(-c6cccc7ccccc67)cc5)cc4)c4ccc(-c5cccc6c5ccc5ccccc56)cc4)c3)c21 ; CC1(C)c2ccccc2-c2c(N(c3ccc(-c4ccc(-c5cccc6ccccc56)cc4)cc3)c3ccc(-c4cccc5c4ccc4ccccc45)cc3)cccc21 ; CC1(C)c2ccccc2-c2c(-c3cccc(N(c4ccc(-c5ccc(-c6cccc7ccccc67)cc5)cc4)c4ccc(-c5cccc6c5ccc5ccccc56)cc4)c3)cccc21]

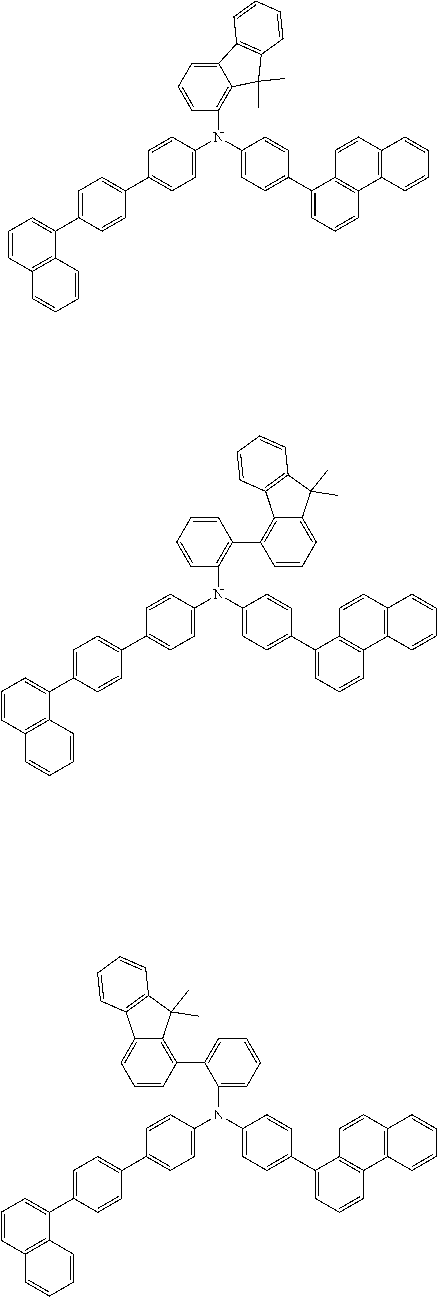 [SMILES: CC1(C)c2ccccc2-c2cccc(N(c3ccc(-c4ccc(-c5cccc6ccccc56)cc4)cc3)c3ccc(-c4cccc5c4ccc4ccccc45)cc3)c21 ; CC1(C)c2ccccc2-c2c(-c3ccccc3N(c3ccc(-c4ccc(-c5cccc6ccccc56)cc4)cc3)c3ccc(-c4cccc5c4ccc4ccccc45)cc3)cccc21 ; CC1(C)c2ccccc2-c2cccc(-c3ccccc3N(c3ccc(-c4ccc(-c5cccc6ccccc56)cc4)cc3)c3ccc(-c4cccc5c4ccc4ccccc45)cc3)c21]

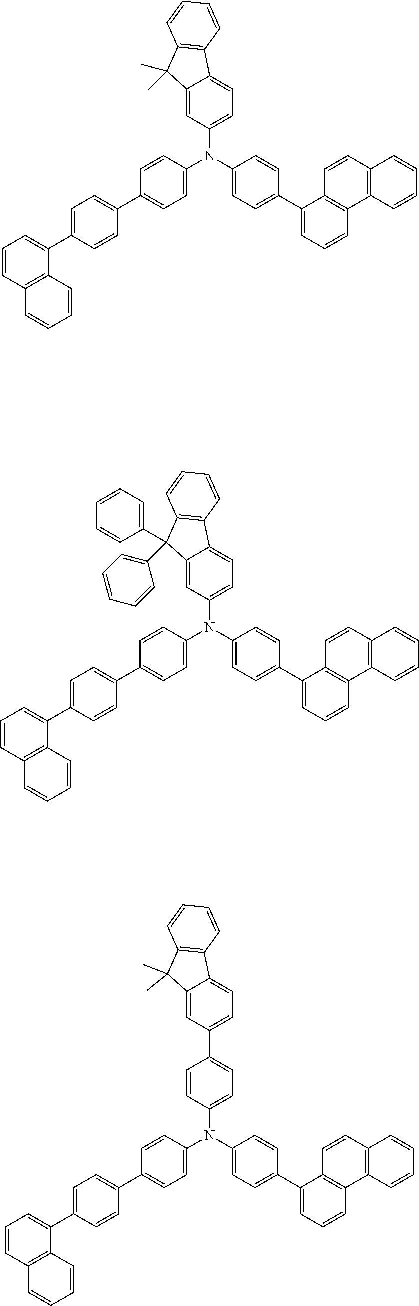 [SMILES: CC1(C)c2ccccc2-c2ccc(N(c3ccc(-c4ccc(-c5cccc6ccccc56)cc4)cc3)c3ccc(-c4cccc5c4ccc4ccccc45)cc3)cc21 ; c1ccc(C2(c3ccccc3)c3ccccc3-c3ccc(N(c4ccc(-c5ccc(-c6cccc7ccccc67)cc5)cc4)c4ccc(-c5cccc6c5ccc5ccccc56)cc4)cc32)cc1 ; CC1(C)c2ccccc2-c2ccc(-c3ccc(N(c4ccc(-c5ccc(-c6cccc7ccccc67)cc5)cc4)c4ccc(-c5cccc6c5ccc5ccccc56)cc4)cc3)cc21]

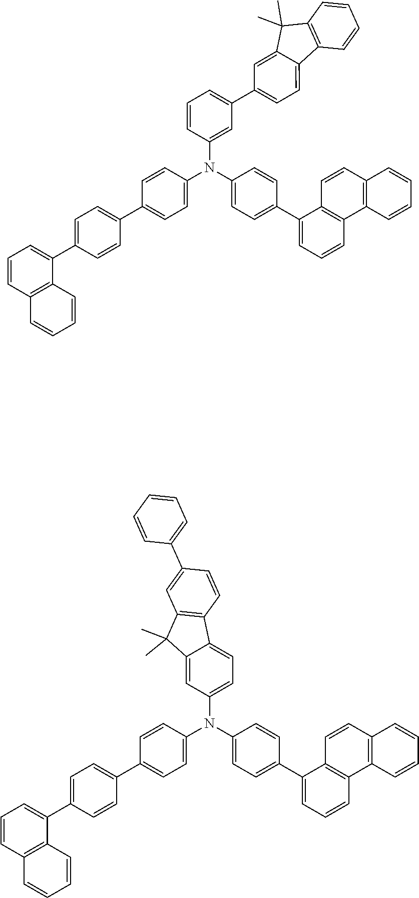 [SMILES: CC1(C)c2ccccc2-c2ccc(-c3cccc(N(c4ccc(-c5ccc(-c6cccc7ccccc67)cc5)cc4)c4ccc(-c5cccc6c5ccc5ccccc56)cc4)c3)cc21 ; CC1(C)c2cc(-c3ccccc3)ccc2-c2ccc(N(c3ccc(-c4ccc(-c5cccc6ccccc56)cc4)cc3)c3ccc(-c4cccc5c4ccc4ccccc45)cc3)cc21]

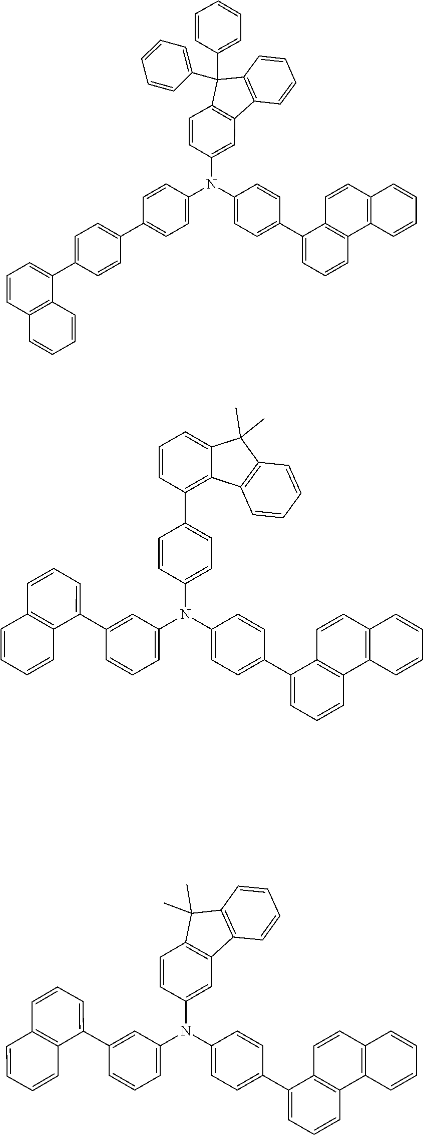 [SMILES: c1ccc(C2(c3ccccc3)c3ccccc3-c3cc(N(c4ccc(-c5ccc(-c6cccc7ccccc67)cc5)cc4)c4ccc(-c5cccc6c5ccc5ccccc56)cc4)ccc32)cc1 ; CC1(C)c2ccccc2-c2c(-c3ccc(N(c4ccc(-c5cccc6c5ccc5ccccc56)cc4)c4cccc(-c5cccc6ccccc56)c4)cc3)cccc21 ; CC1(C)c2ccccc2-c2cc(N(c3ccc(-c4cccc5c4ccc4ccccc45)cc3)c3cccc(-c4cccc5ccccc45)c3)ccc21]

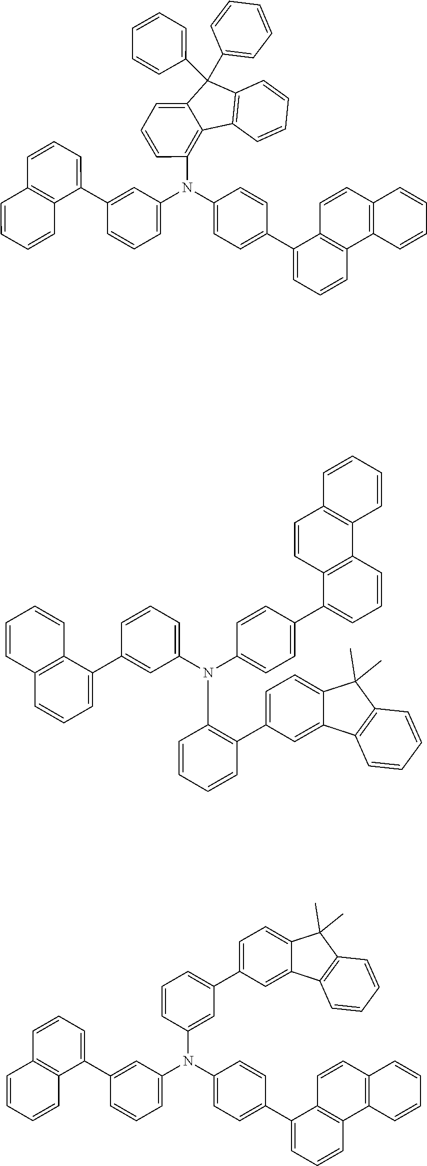 [SMILES: c1ccc(C2(c3ccccc3)c3ccccc3-c3c(N(c4ccc(-c5cccc6c5ccc5ccccc56)cc4)c4cccc(-c5cccc6ccccc56)c4)cccc32)cc1 ; CC1(C)c2ccccc2-c2cc(-c3ccccc3N(c3ccc(-c4cccc5c4ccc4ccccc45)cc3)c3cccc(-c4cccc5ccccc45)c3)ccc21 ; CC1(C)c2ccccc2-c2cc(-c3cccc(N(c4ccc(-c5cccc6c5ccc5ccccc56)cc4)c4cccc(-c5cccc6ccccc56)c4)c3)ccc21]

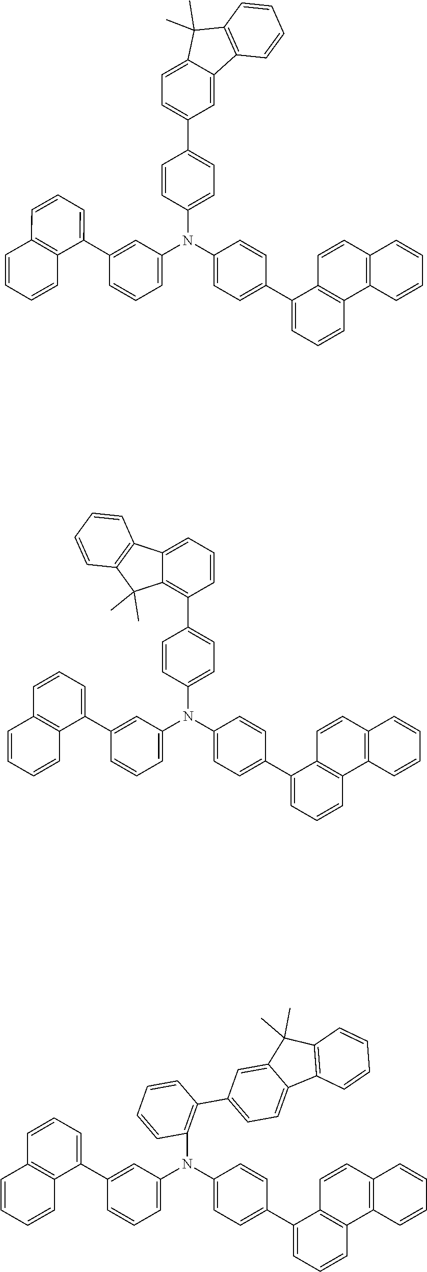 [SMILES: CC1(C)c2ccccc2-c2cc(-c3ccc(N(c4ccc(-c5cccc6c5ccc5ccccc56)cc4)c4cccc(-c5cccc6ccccc56)c4)cc3)ccc21 ; CC1(C)c2ccccc2-c2cccc(-c3ccc(N(c4ccc(-c5cccc6c5ccc5ccccc56)cc4)c4cccc(-c5cccc6ccccc56)c4)cc3)c21 ; CC1(C)c2ccccc2-c2ccc(-c3ccccc3N(c3ccc(-c4cccc5c4ccc4ccccc45)cc3)c3cccc(-c4cccc5ccccc45)c3)cc21]

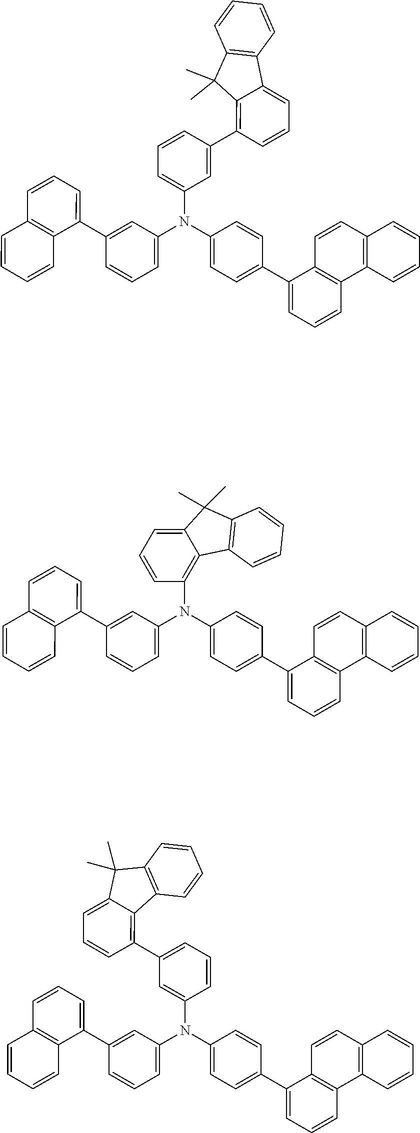 [SMILES: CC1(C)c2ccccc2-c2cccc(-c3cccc(N(c4ccc(-c5cccc6c5ccc5ccccc56)cc4)c4cccc(-c5cccc6ccccc56)c4)c3)c21 ; CC1(C)c2ccccc2-c2c(N(c3ccc(-c4cccc5c4ccc4ccccc45)cc3)c3cccc(-c4cccc5ccccc45)c3)cccc21 ; CC1(C)c2ccccc2-c2c(-c3cccc(N(c4ccc(-c5cccc6c5ccc5ccccc56)cc4)c4cccc(-c5cccc6ccccc56)c4)c3)cccc21]

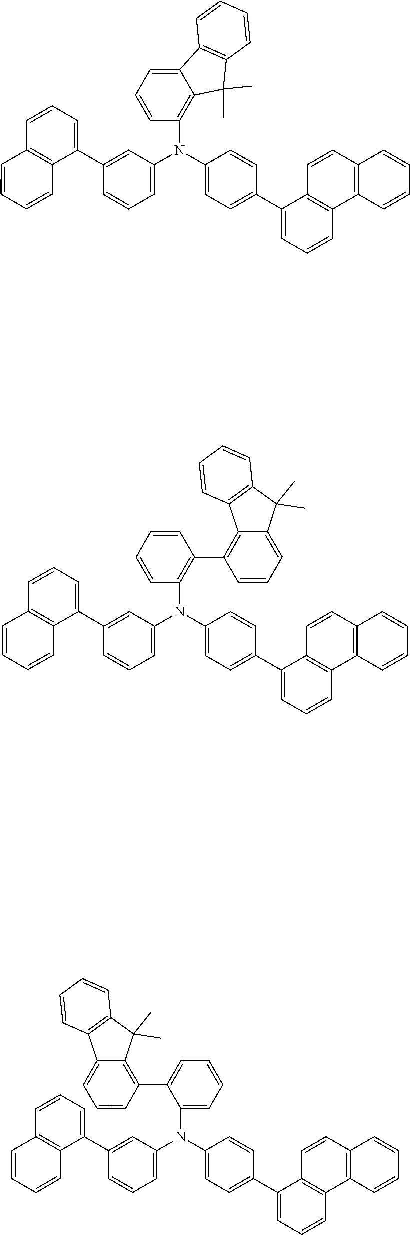 [SMILES: CC1(C)c2ccccc2-c2cccc(N(c3ccc(-c4cccc5c4ccc4ccccc45)cc3)c3cccc(-c4cccc5ccccc45)c3)c21 ; CC1(C)c2ccccc2-c2c(-c3ccccc3N(c3ccc(-c4cccc5c4ccc4ccccc45)cc3)c3cccc(-c4cccc5ccccc45)c3)cccc21 ; CC1(C)c2ccccc2-c2cccc(-c3ccccc3N(c3ccc(-c4cccc5c4ccc4ccccc45)cc3)c3cccc(-c4cccc5ccccc45)c3)c21]

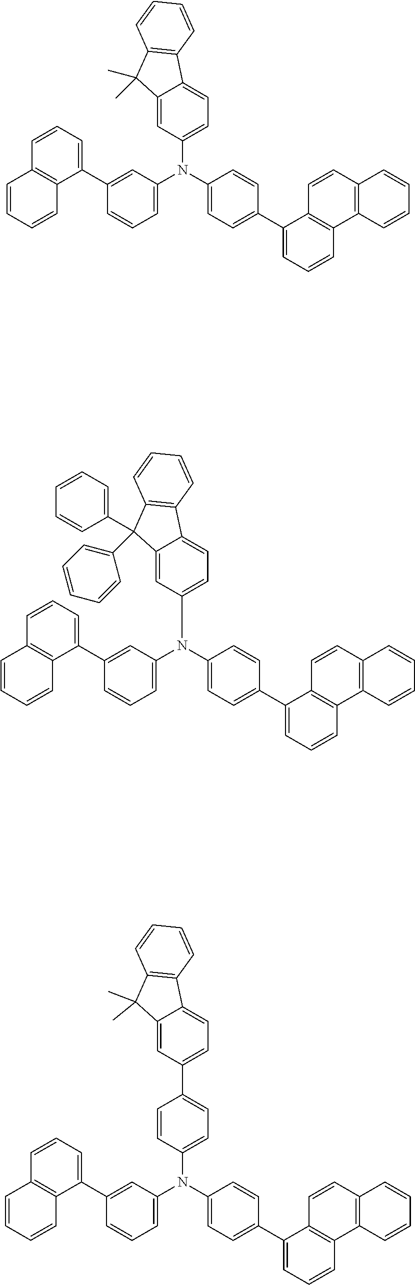 [SMILES: CC1(C)c2ccccc2-c2ccc(N(c3ccc(-c4cccc5c4ccc4ccccc45)cc3)c3cccc(-c4cccc5ccccc45)c3)cc21 ; c1ccc(C2(c3ccccc3)c3ccccc3-c3ccc(N(c4ccc(-c5cccc6c5ccc5ccccc56)cc4)c4cccc(-c5cccc6ccccc56)c4)cc32)cc1 ; CC1(C)c2ccccc2-c2ccc(-c3ccc(N(c4ccc(-c5cccc6c5ccc5ccccc56)cc4)c4cccc(-c5cccc6ccccc56)c4)cc3)cc21]

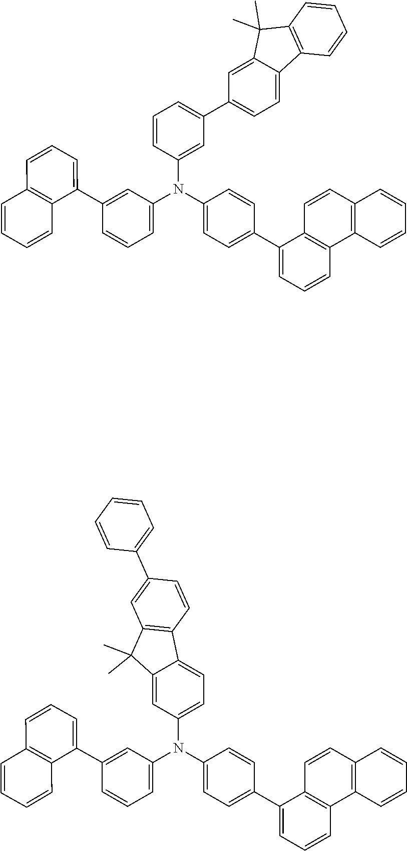 [SMILES: CC1(C)c2ccccc2-c2ccc(-c3cccc(N(c4ccc(-c5cccc6c5ccc5ccccc56)cc4)c4cccc(-c5cccc6ccccc56)c4)c3)cc21 ; CC1(C)c2cc(-c3ccccc3)ccc2-c2ccc(N(c3ccc(-c4cccc5c4ccc4ccccc45)cc3)c3cccc(-c4cccc5ccccc45)c3)cc21]

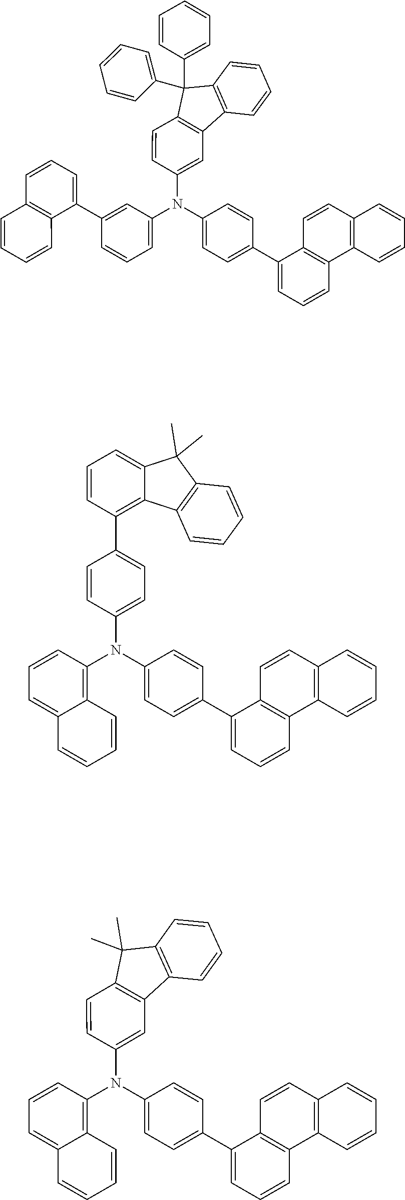 [SMILES: c1ccc(C2(c3ccccc3)c3ccccc3-c3cc(N(c4ccc(-c5cccc6c5ccc5ccccc56)cc4)c4cccc(-c5cccc6ccccc56)c4)ccc32)cc1 ; CC1(C)c2ccccc2-c2c(-c3ccc(N(c4ccc(-c5cccc6c5ccc5ccccc56)cc4)c4cccc5ccccc45)cc3)cccc21 ; CC1(C)c2ccccc2-c2cc(N(c3ccc(-c4cccc5c4ccc4ccccc45)cc3)c3cccc4ccccc34)ccc21]

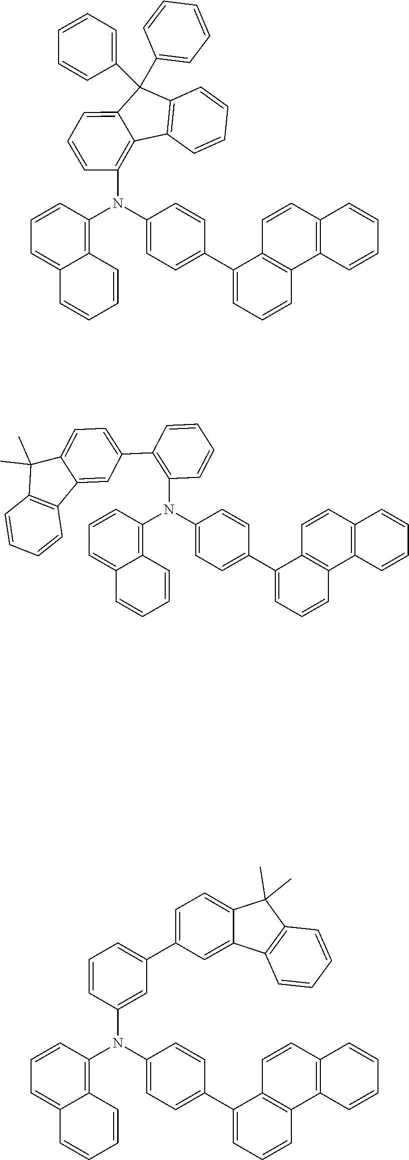 [SMILES: c1ccc(C2(c3ccccc3)c3ccccc3-c3c(N(c4ccc(-c5cccc6c5ccc5ccccc56)cc4)c4cccc5ccccc45)cccc32)cc1 ; CC1(C)c2ccccc2-c2cc(-c3ccccc3N(c3ccc(-c4cccc5c4ccc4ccccc45)cc3)c3cccc4ccccc34)ccc21 ; CC1(C)c2ccccc2-c2cc(-c3cccc(N(c4ccc(-c5cccc6c5ccc5ccccc56)cc4)c4cccc5ccccc45)c3)ccc21]

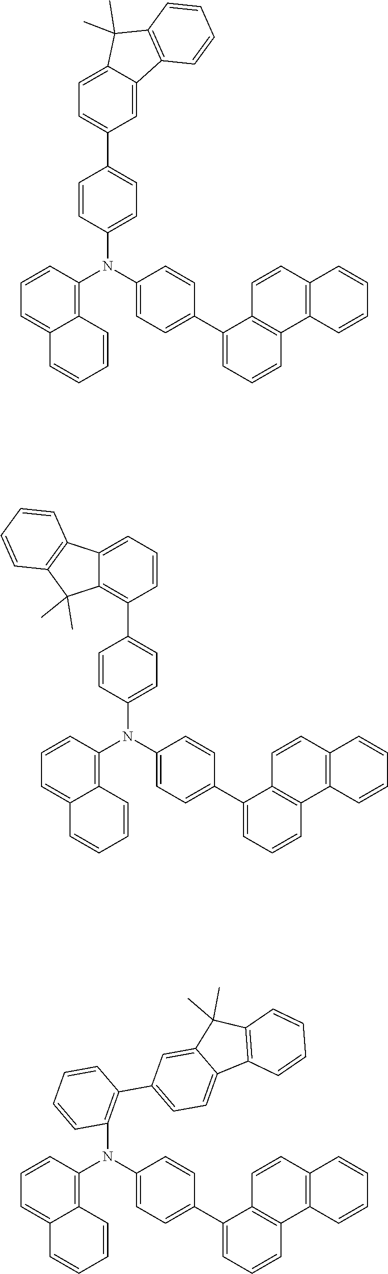 [SMILES: CC1(C)c2ccccc2-c2cc(-c3ccc(N(c4ccc(-c5cccc6c5ccc5ccccc56)cc4)c4cccc5ccccc45)cc3)ccc21 ; CC1(C)c2ccccc2-c2cccc(-c3ccc(N(c4ccc(-c5cccc6c5ccc5ccccc56)cc4)c4cccc5ccccc45)cc3)c21 ; CC1(C)c2ccccc2-c2ccc(-c3ccccc3N(c3ccc(-c4cccc5c4ccc4ccccc45)cc3)c3cccc4ccccc34)cc21]

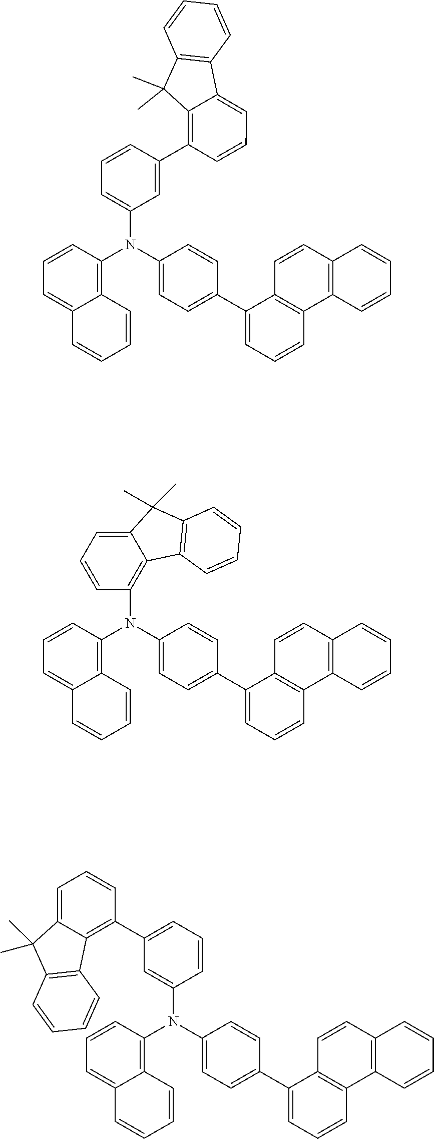 [SMILES: CC1(C)c2ccccc2-c2cccc(-c3cccc(N(c4ccc(-c5cccc6c5ccc5ccccc56)cc4)c4cccc5ccccc45)c3)c21 ; CC1(C)c2ccccc2-c2c(N(c3ccc(-c4cccc5c4ccc4ccccc45)cc3)c3cccc4ccccc34)cccc21 ; CC1(C)c2ccccc2-c2c(-c3cccc(N(c4ccc(-c5cccc6c5ccc5ccccc56)cc4)c4cccc5ccccc45)c3)cccc21]

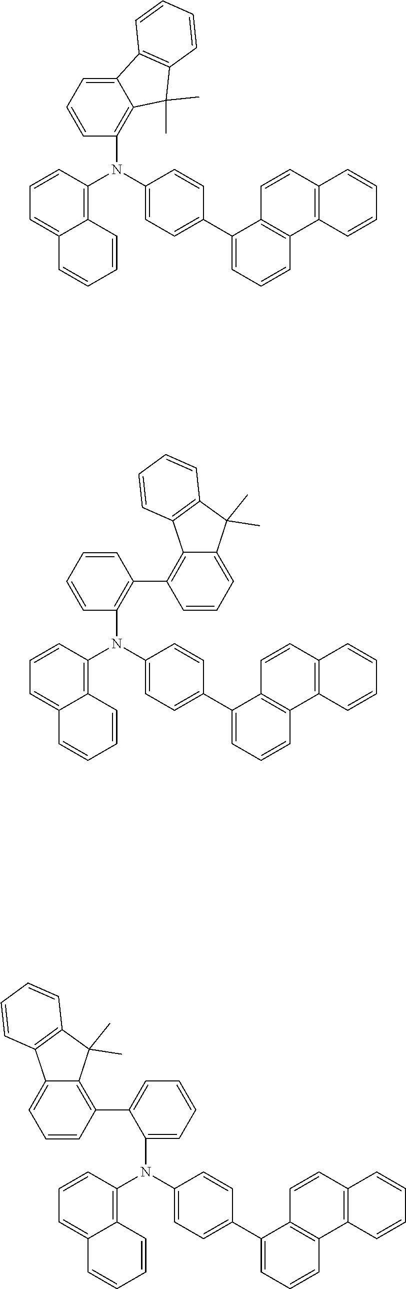 [SMILES: CC1(C)c2ccccc2-c2cccc(N(c3ccc(-c4cccc5c4ccc4ccccc45)cc3)c3cccc4ccccc34)c21 ; CC1(C)c2ccccc2-c2c(-c3ccccc3N(c3ccc(-c4cccc5c4ccc4ccccc45)cc3)c3cccc4ccccc34)cccc21 ; CC1(C)c2ccccc2-c2cccc(-c3ccccc3N(c3ccc(-c4cccc5c4ccc4ccccc45)cc3)c3cccc4ccccc34)c21]

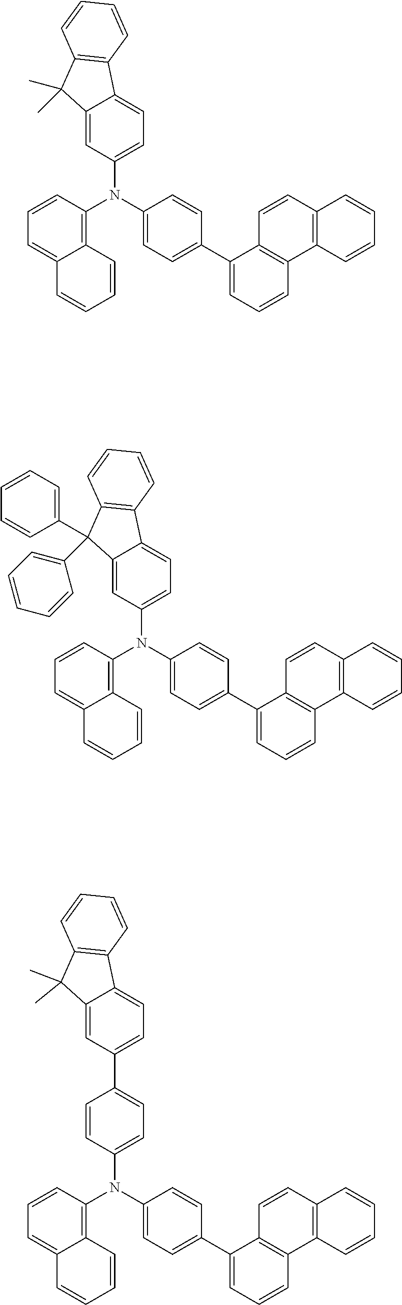 [SMILES: CC1(C)c2ccccc2-c2ccc(N(c3ccc(-c4cccc5c4ccc4ccccc45)cc3)c3cccc4ccccc34)cc21 ; c1ccc(C2(c3ccccc3)c3ccccc3-c3ccc(N(c4ccc(-c5cccc6c5ccc5ccccc56)cc4)c4cccc5ccccc45)cc32)cc1 ; CC1(C)c2ccccc2-c2ccc(-c3ccc(N(c4ccc(-c5cccc6c5ccc5ccccc56)cc4)c4cccc5ccccc45)cc3)cc21]

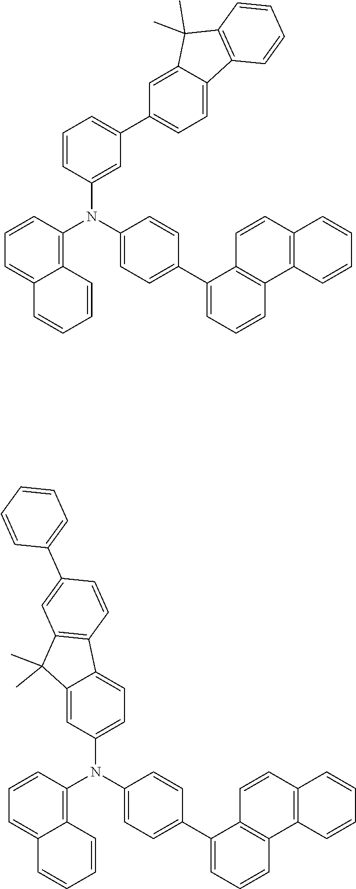 [SMILES: CC1(C)c2ccccc2-c2ccc(-c3cccc(N(c4ccc(-c5cccc6c5ccc5ccccc56)cc4)c4cccc5ccccc45)c3)cc21 ; CC1(C)c2cc(-c3ccccc3)ccc2-c2ccc(N(c3ccc(-c4cccc5c4ccc4ccccc45)cc3)c3cccc4ccccc34)cc21]

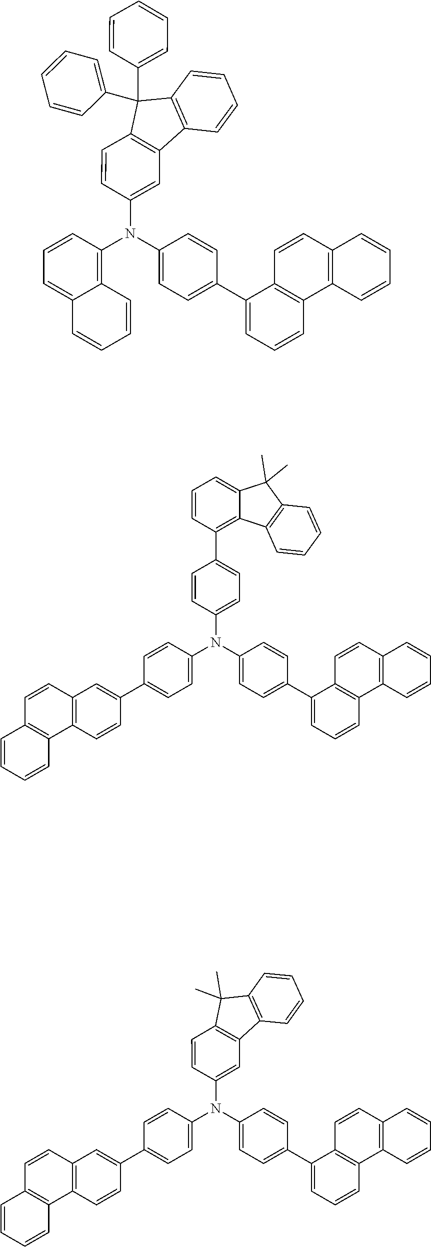 [SMILES: c1ccc(C2(c3ccccc3)c3ccccc3-c3cc(N(c4ccc(-c5cccc6c5ccc5ccccc56)cc4)c4cccc5ccccc45)ccc32)cc1 ; CC1(C)c2ccccc2-c2c(-c3ccc(N(c4ccc(-c5ccc6c(ccc7ccccc76)c5)cc4)c4ccc(-c5cccc6c5ccc5ccccc56)cc4)cc3)cccc21 ; CC1(C)c2ccccc2-c2cc(N(c3ccc(-c4ccc5c(ccc6ccccc65)c4)cc3)c3ccc(-c4cccc5c4ccc4ccccc45)cc3)ccc21]

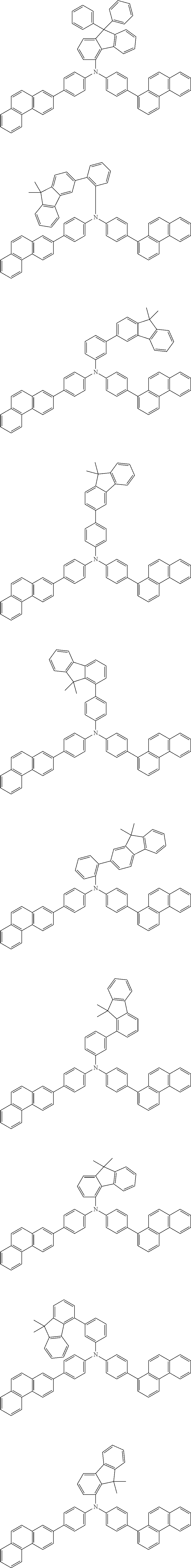

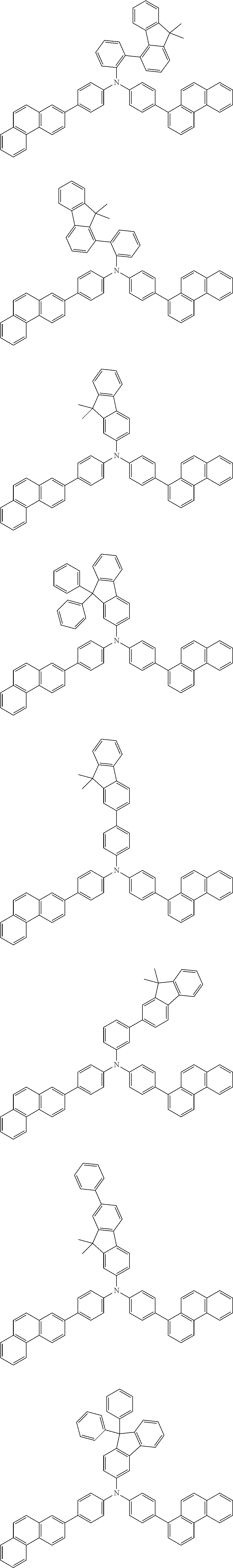

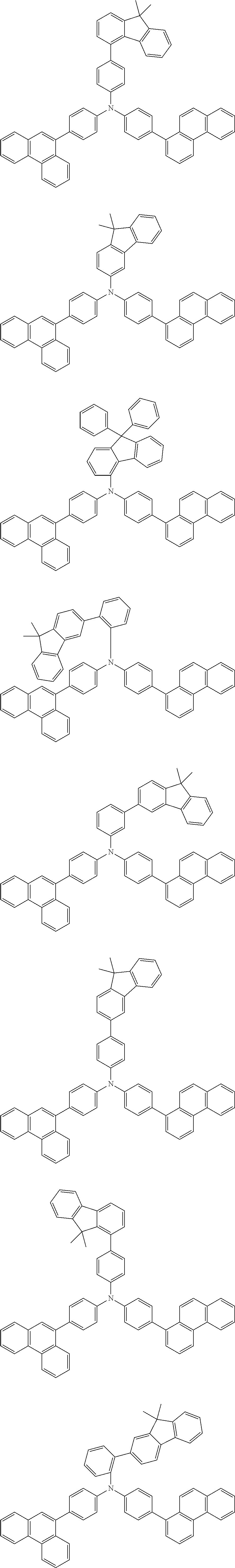

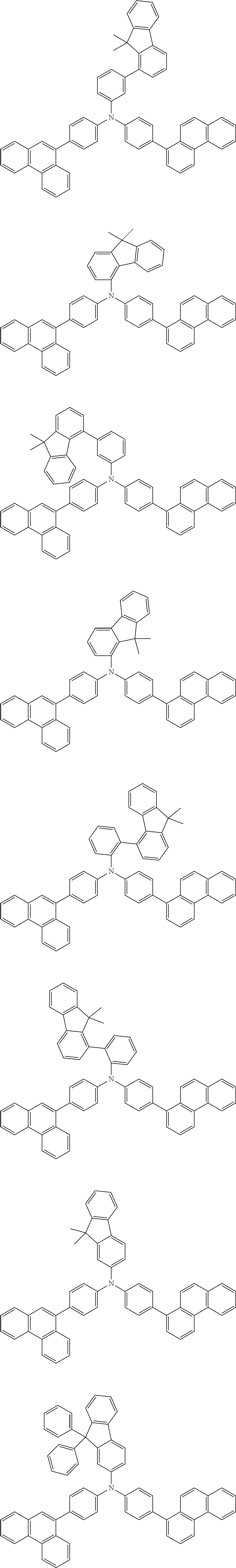

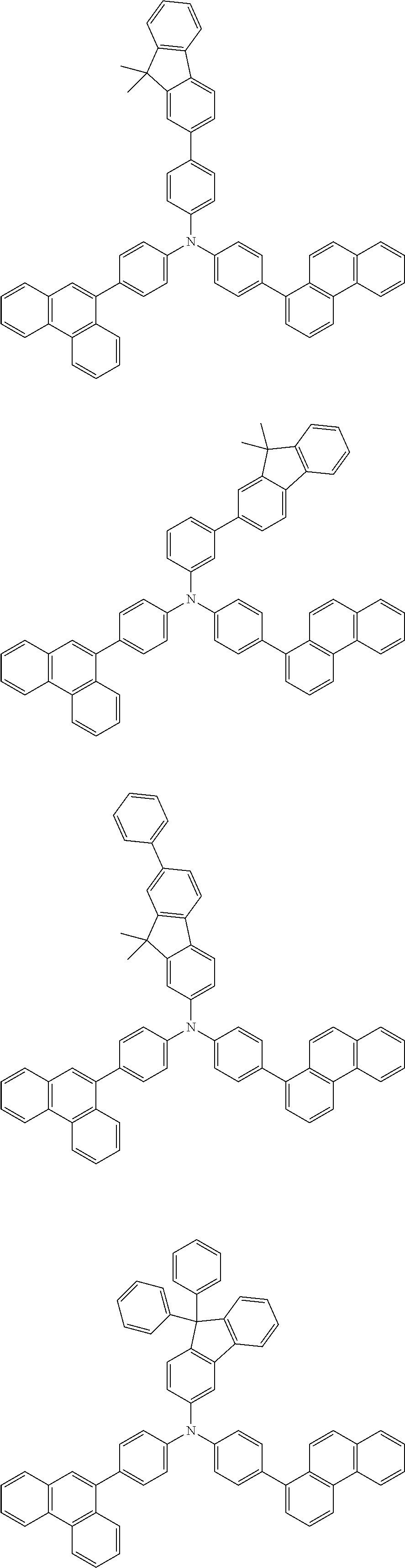 [SMILES: CC1(C)c2ccccc2-c2ccc(-c3ccc(N(c4ccc(-c5cccc6c5ccc5ccccc56)cc4)c4ccc(-c5cc6ccccc6c6ccccc56)cc4)cc3)cc21 ; CC1(C)c2ccccc2-c2ccc(-c3cccc(N(c4ccc(-c5cccc6c5ccc5ccccc56)cc4)c4ccc(-c5cc6ccccc6c6ccccc56)cc4)c3)cc21 ; CC1(C)c2cc(-c3ccccc3)ccc2-c2ccc(N(c3ccc(-c4cccc5c4ccc4ccccc45)cc3)c3ccc(-c4cc5ccccc5c5ccccc45)cc3)cc21 ; c1ccc(C2(c3ccccc3)c3ccccc3-c3cc(N(c4ccc(-c5cccc6c5ccc5ccccc56)cc4)c4ccc(-c5cc6ccccc6c6ccccc56)cc4)ccc32)cc1]

[chemical structure]

[chemical structure]

[chemical structure]

[chemical structure]

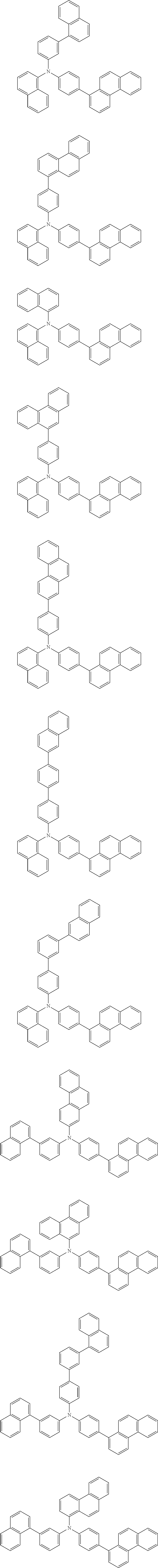

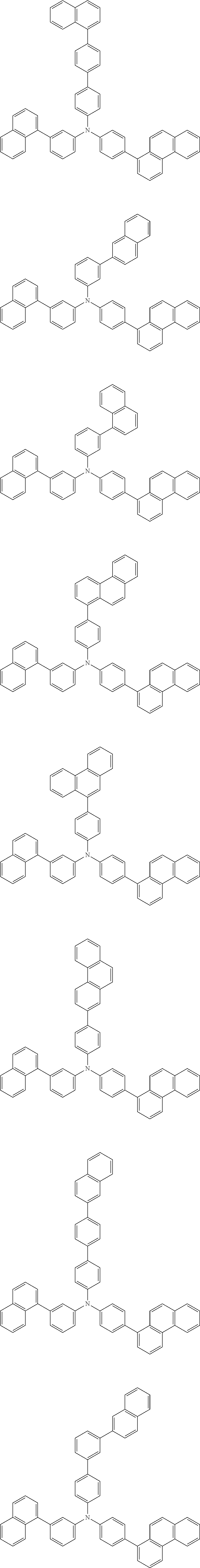

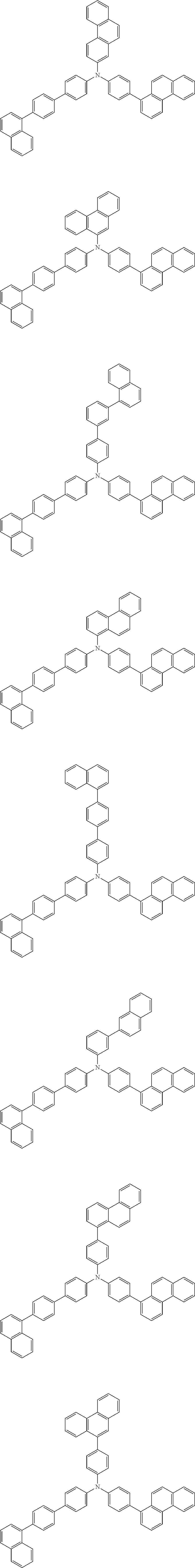

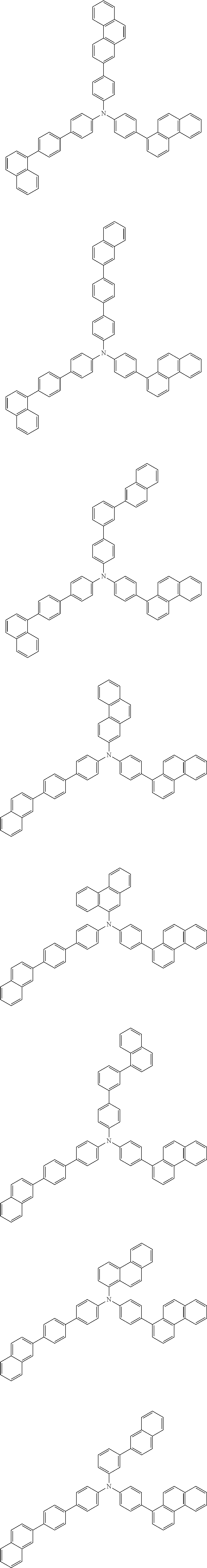

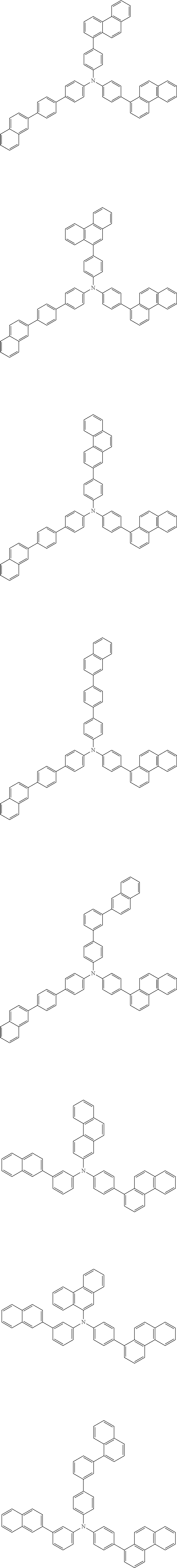

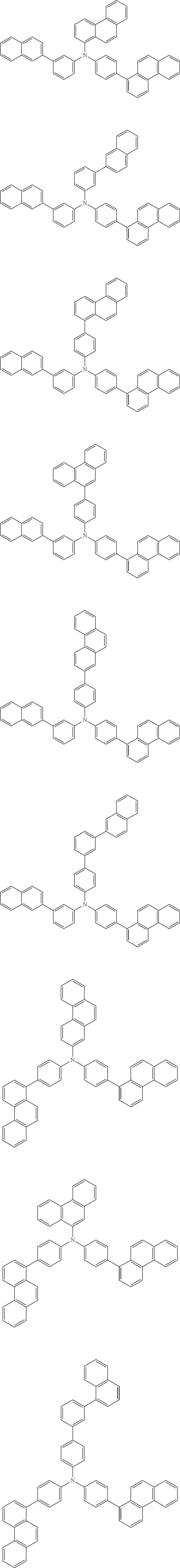

[chemical structure]

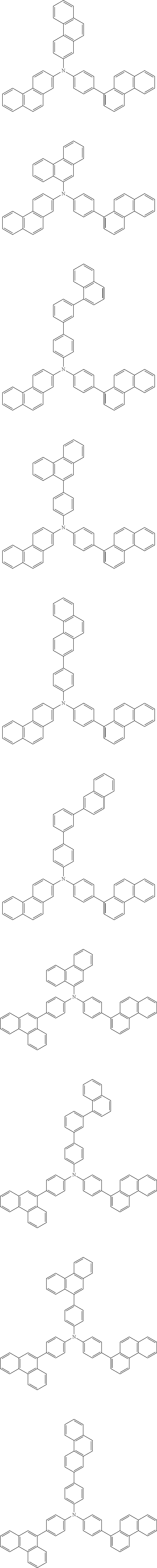

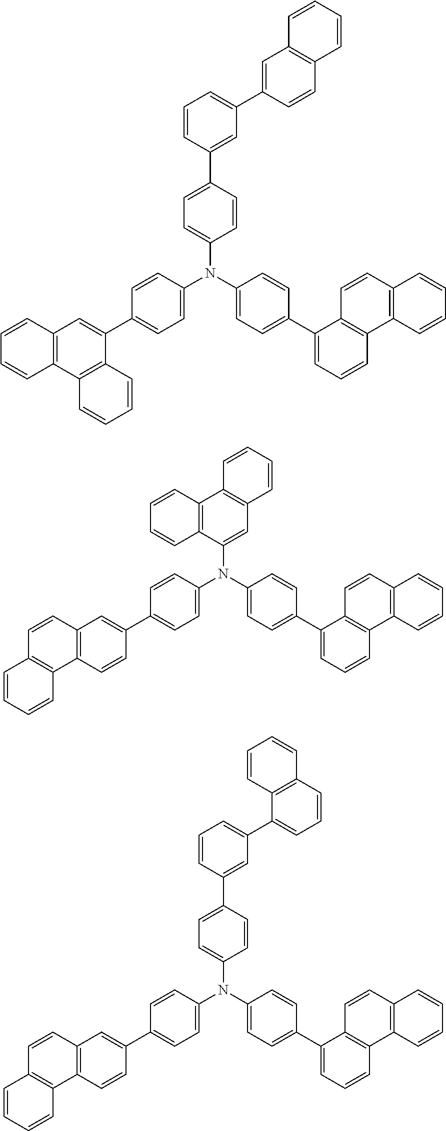 [SMILES: c1cc(-c2ccc(N(c3ccc(-c4cccc5c4ccc4ccccc45)cc3)c3ccc(-c4cc5ccccc5c5ccccc45)cc3)cc2)cc(-c2ccc3ccccc3c2)c1 ; c1ccc2c(c1)ccc1cc(-c3ccc(N(c4ccc(-c5cccc6c5ccc5ccccc56)cc4)c4cc5ccccc5c5ccccc45)cc3)ccc12 ; c1cc(-c2ccc(N(c3ccc(-c4ccc5c(ccc6ccccc65)c4)cc3)c3ccc(-c4cccc5c4ccc4ccccc45)cc3)cc2)cc(-c2cccc3ccccc23)c1]

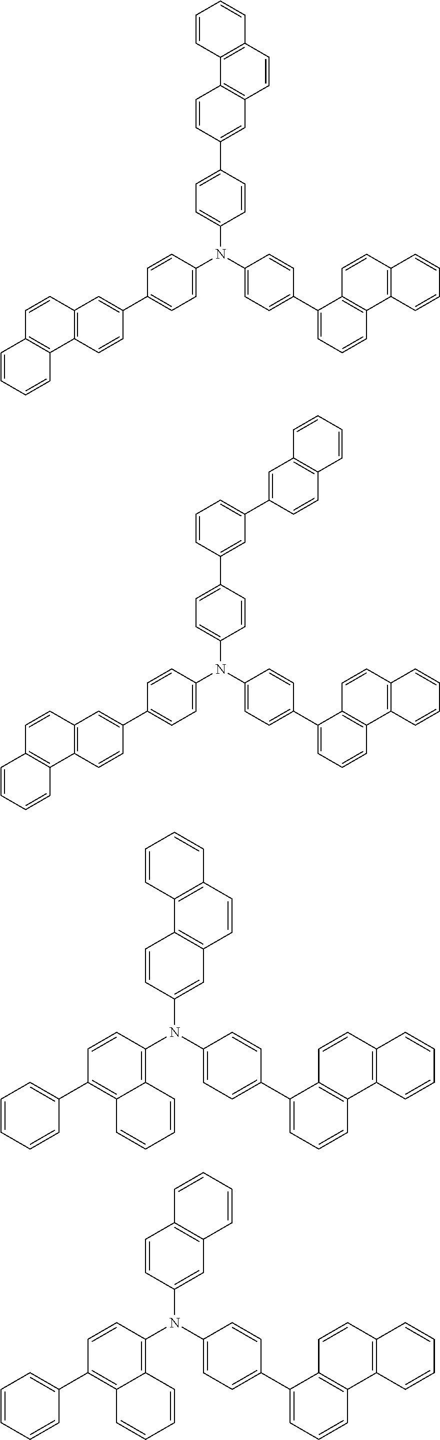 [SMILES: c1ccc2c(c1)ccc1cc(-c3ccc(N(c4ccc(-c5ccc6c(ccc7ccccc76)c5)cc4)c4ccc(-c5cccc6c5ccc5ccccc56)cc4)cc3)ccc12 ; c1cc(-c2ccc(N(c3ccc(-c4ccc5c(ccc6ccccc65)c4)cc3)c3ccc(-c4cccc5c4ccc4ccccc45)cc3)cc2)cc(-c2ccc3ccccc3c2)c1 ; c1ccc(-c2ccc(N(c3ccc(-c4cccc5c4ccc4ccccc45)cc3)c3ccc4c(ccc5ccccc54)c3)c3ccccc23)cc1 ; c1ccc(-c2ccc(N(c3ccc(-c4cccc5c4ccc4ccccc45)cc3)c3ccc4ccccc4c3)c3ccccc23)cc1]

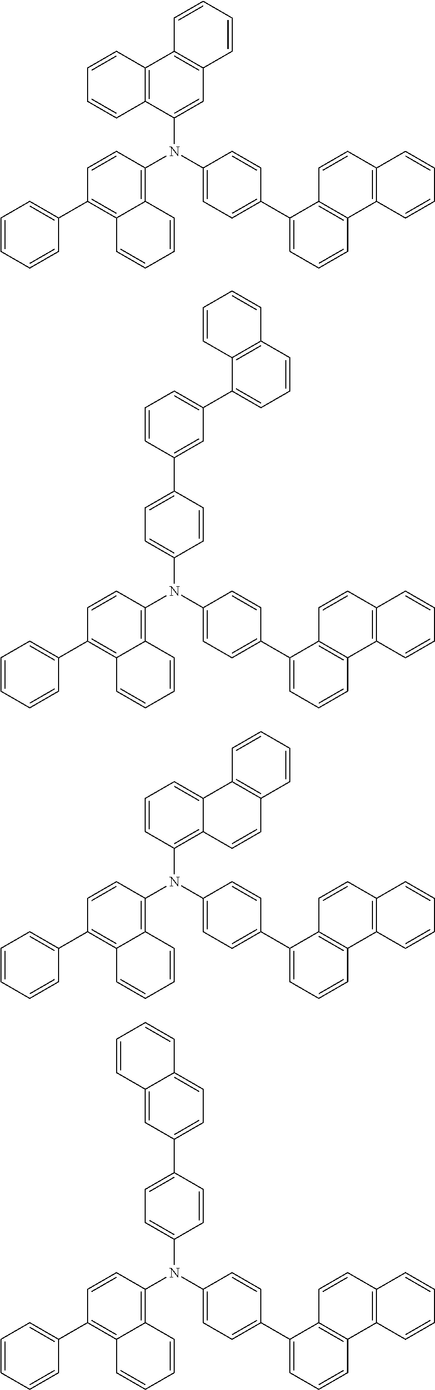

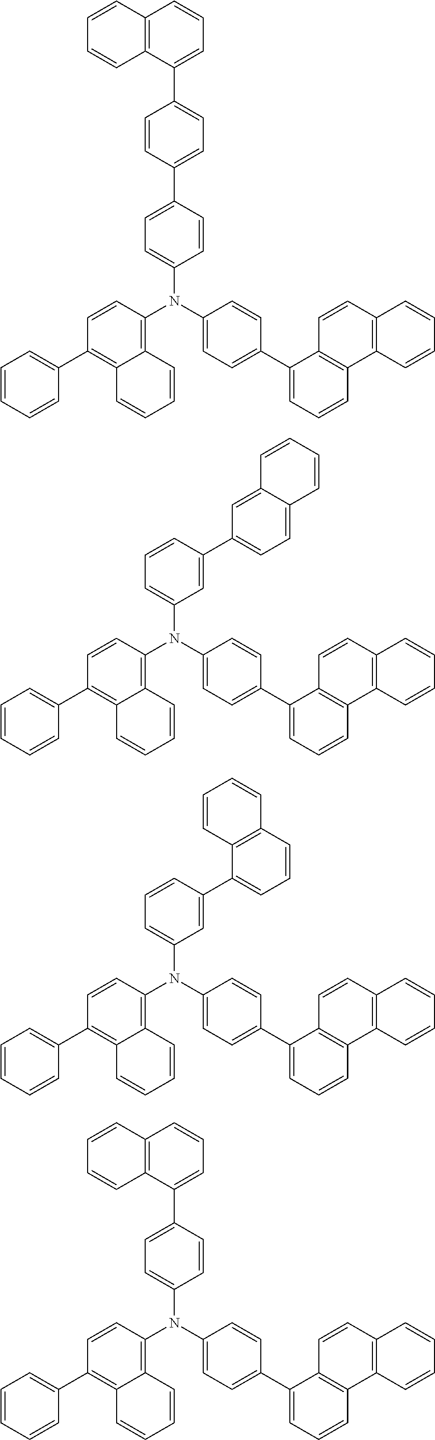

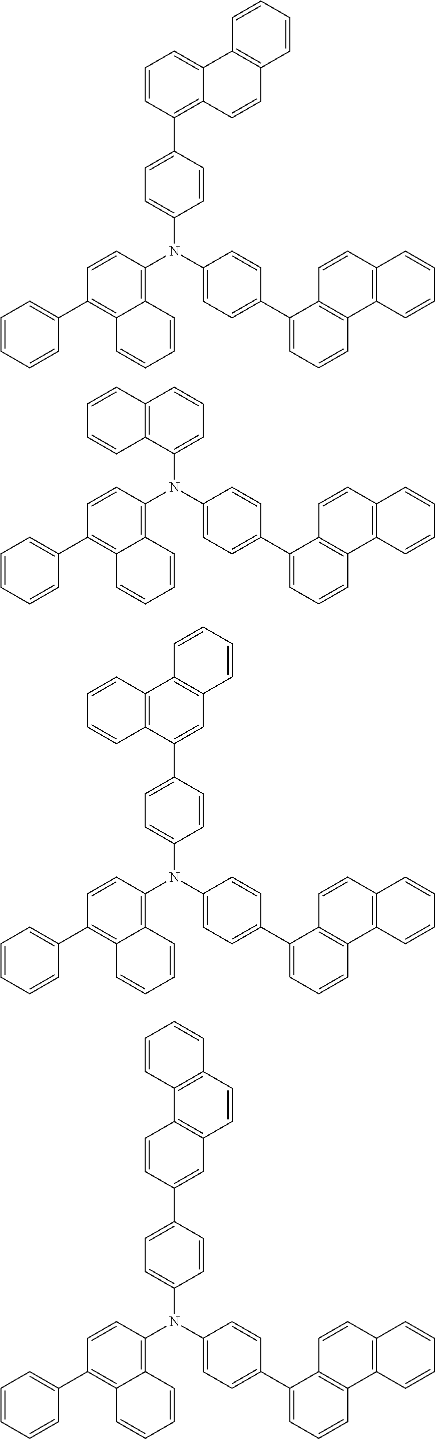

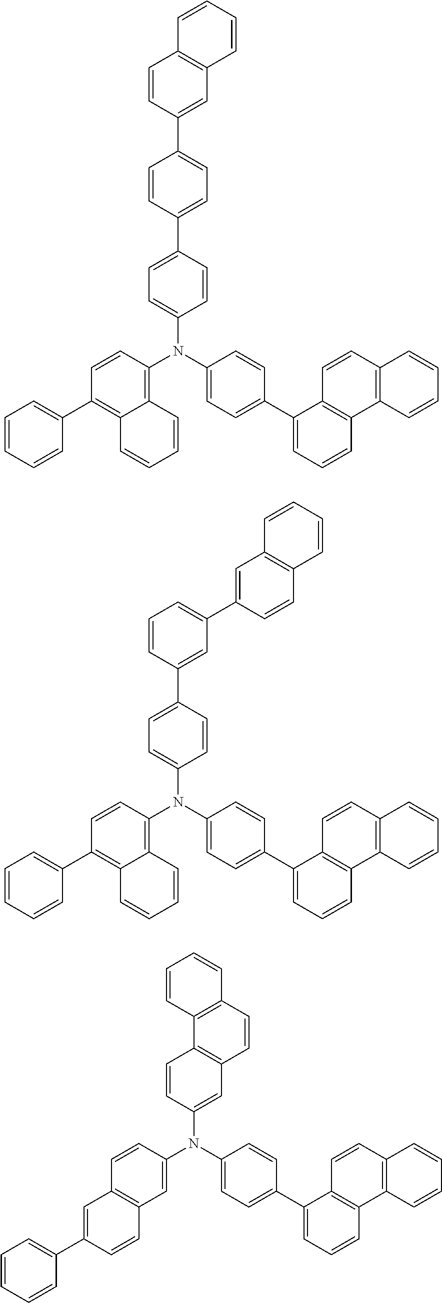

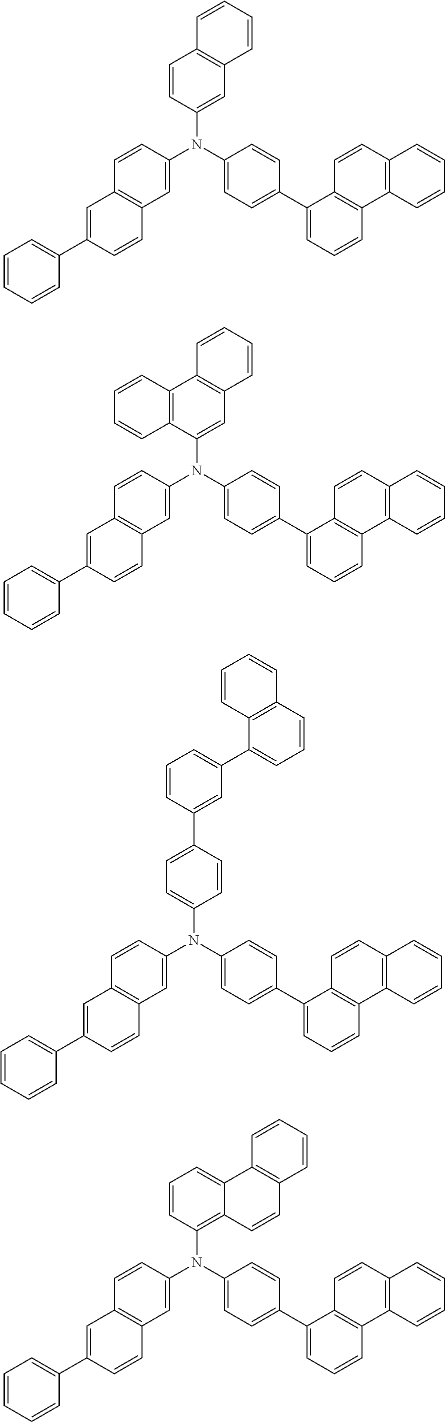 [SMILES: c1ccc(-c2ccc3cc(N(c4ccc(-c5cccc6c5ccc5ccccc56)cc4)c4ccc5ccccc5c4)ccc3c2)cc1 ; c1ccc(-c2ccc3cc(N(c4ccc(-c5cccc6c5ccc5ccccc56)cc4)c4cc5ccccc5c5ccccc45)ccc3c2)cc1 ; c1ccc(-c2ccc3cc(N(c4ccc(-c5cccc(-c6cccc7ccccc67)c5)cc4)c4ccc(-c5cccc6c5ccc5ccccc56)cc4)ccc3c2)cc1 ; c1ccc(-c2ccc3cc(N(c4ccc(-c5cccc6c5ccc5ccccc56)cc4)c4cccc5c4ccc4ccccc45)ccc3c2)cc1]

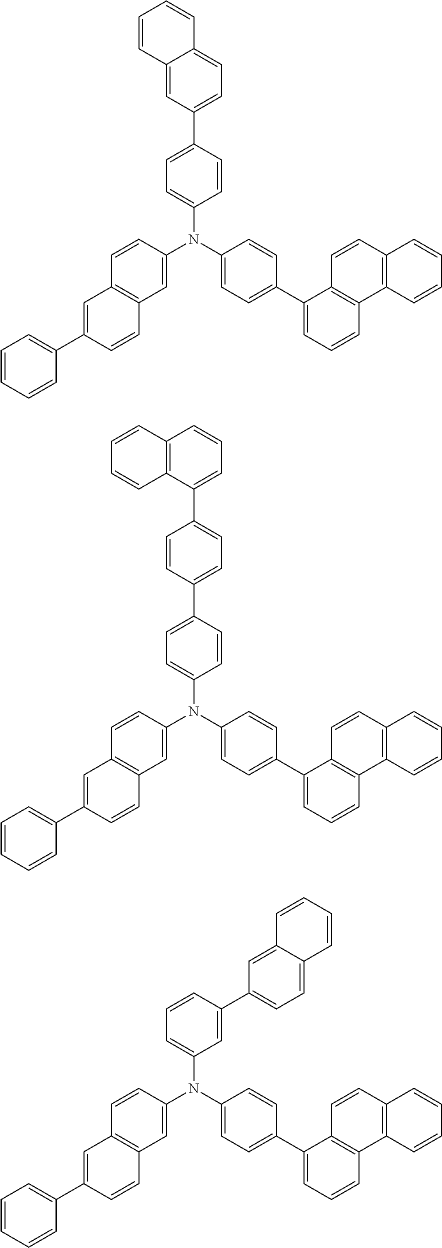 [SMILES: c1ccc(-c2ccc3cc(N(c4ccc(-c5ccc6ccccc6c5)cc4)c4ccc(-c5cccc6c5ccc5ccccc56)cc4)ccc3c2)cc1 ; c1ccc(-c2ccc3cc(N(c4ccc(-c5ccc(-c6cccc7ccccc67)cc5)cc4)c4ccc(-c5cccc6c5ccc5ccccc56)cc4)ccc3c2)cc1 ; c1ccc(-c2ccc3cc(N(c4ccc(-c5cccc6c5ccc5ccccc56)cc4)c4cccc(-c5ccc6ccccc6c5)c4)ccc3c2)cc1]

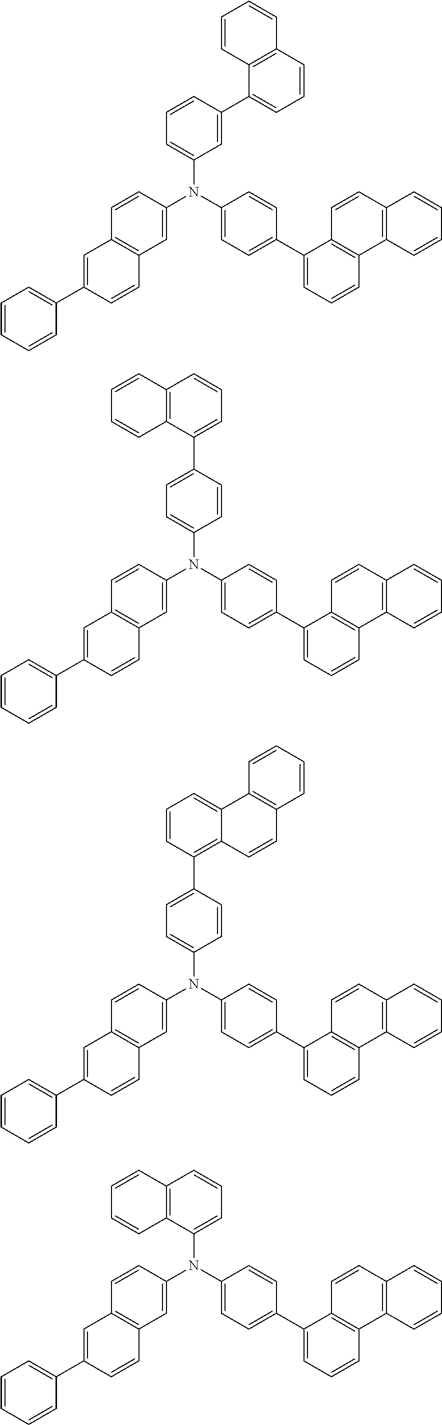 [SMILES: c1ccc(-c2ccc3cc(N(c4ccc(-c5cccc6c5ccc5ccccc56)cc4)c4cccc(-c5cccc6ccccc56)c4)ccc3c2)cc1 ; c1ccc(-c2ccc3cc(N(c4ccc(-c5cccc6ccccc56)cc4)c4ccc(-c5cccc6c5ccc5ccccc56)cc4)ccc3c2)cc1 ; c1ccc(-c2ccc3cc(N(c4ccc(-c5cccc6c5ccc5ccccc56)cc4)c4ccc(-c5cccc6c5ccc5ccccc56)cc4)ccc3c2)cc1 ; c1ccc(-c2ccc3cc(N(c4ccc(-c5cccc6c5ccc5ccccc56)cc4)c4cccc5ccccc45)ccc3c2)cc1]

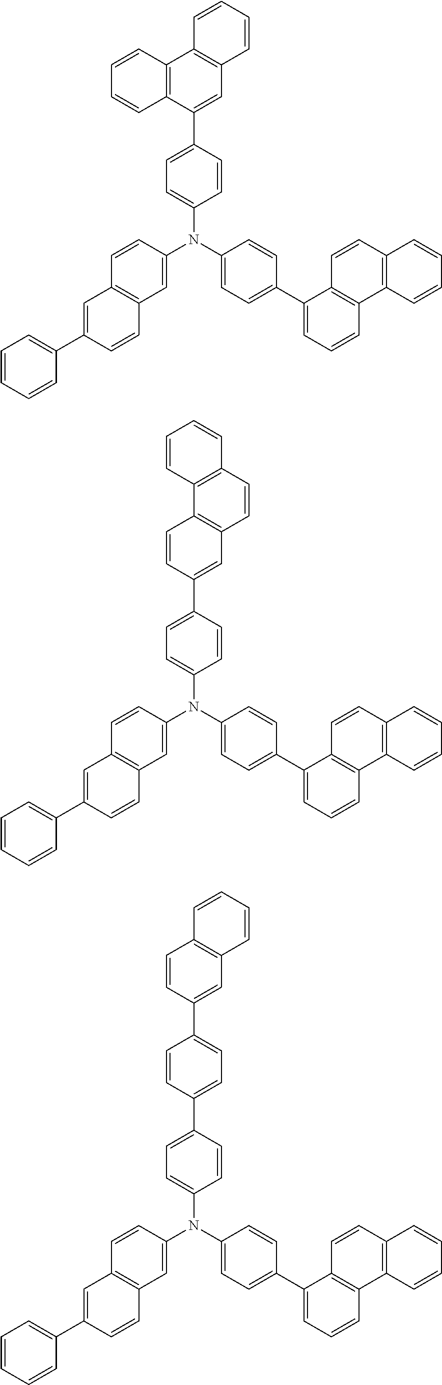 [SMILES: c1ccc(-c2ccc3cc(N(c4ccc(-c5cccc6c5ccc5ccccc56)cc4)c4ccc(-c5cc6ccccc6c6ccccc56)cc4)ccc3c2)cc1 ; c1ccc(-c2ccc3cc(N(c4ccc(-c5ccc6c(ccc7ccccc76)c5)cc4)c4ccc(-c5cccc6c5ccc5ccccc56)cc4)ccc3c2)cc1 ; c1ccc(-c2ccc3cc(N(c4ccc(-c5ccc(-c6ccc7ccccc7c6)cc5)cc4)c4ccc(-c5cccc6c5ccc5ccccc56)cc4)ccc3c2)cc1]

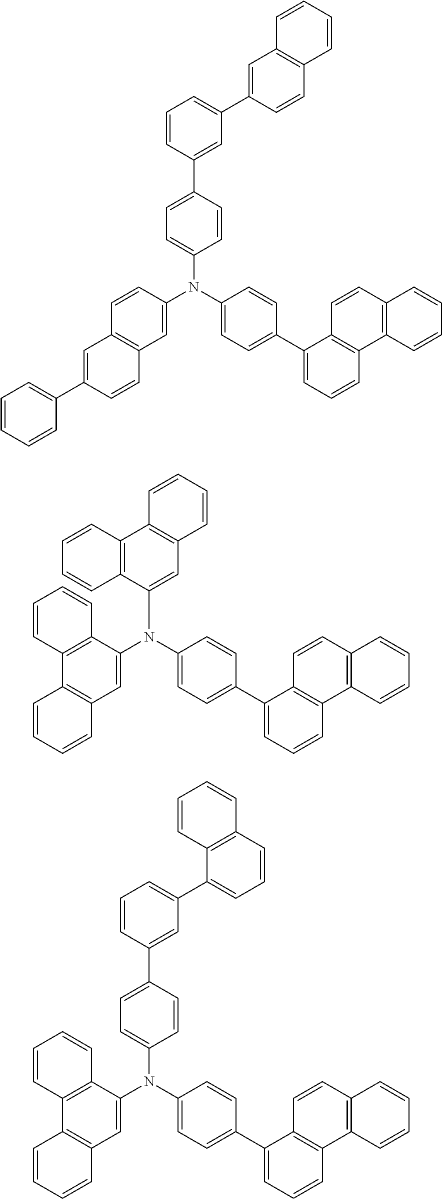 [SMILES: c1ccc(-c2ccc3cc(N(c4ccc(-c5cccc(-c6ccc7ccccc7c6)c5)cc4)c4ccc(-c5cccc6c5ccc5ccccc56)cc4)ccc3c2)cc1 ; c1ccc2c(c1)ccc1c(-c3ccc(N(c4cc5ccccc5c5ccccc45)c4cc5ccccc5c5ccccc45)cc3)cccc12 ; c1cc(-c2ccc(N(c3ccc(-c4cccc5c4ccc4ccccc45)cc3)c3cc4ccccc4c4ccccc34)cc2)cc(-c2cccc3ccccc23)c1]

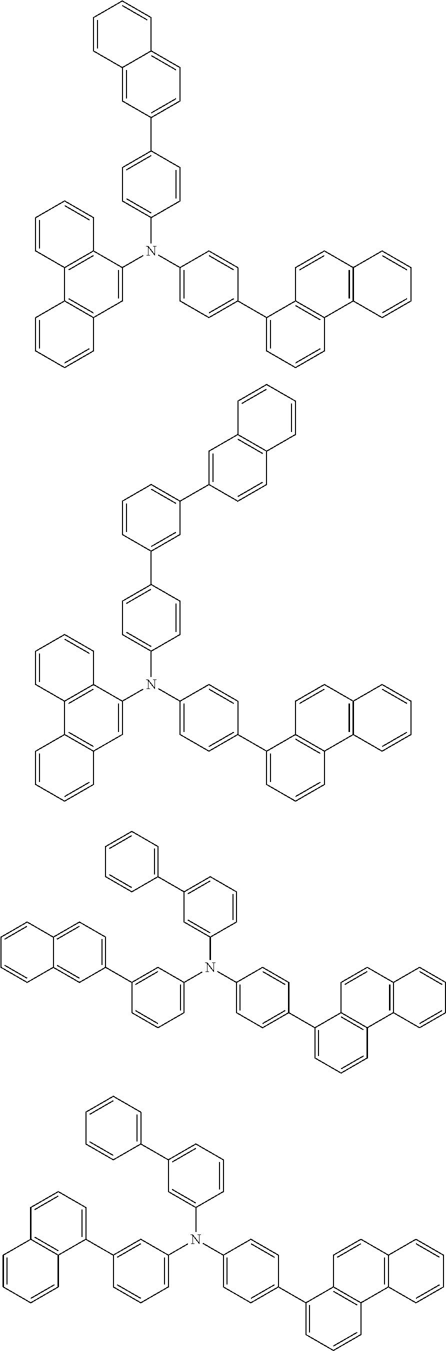

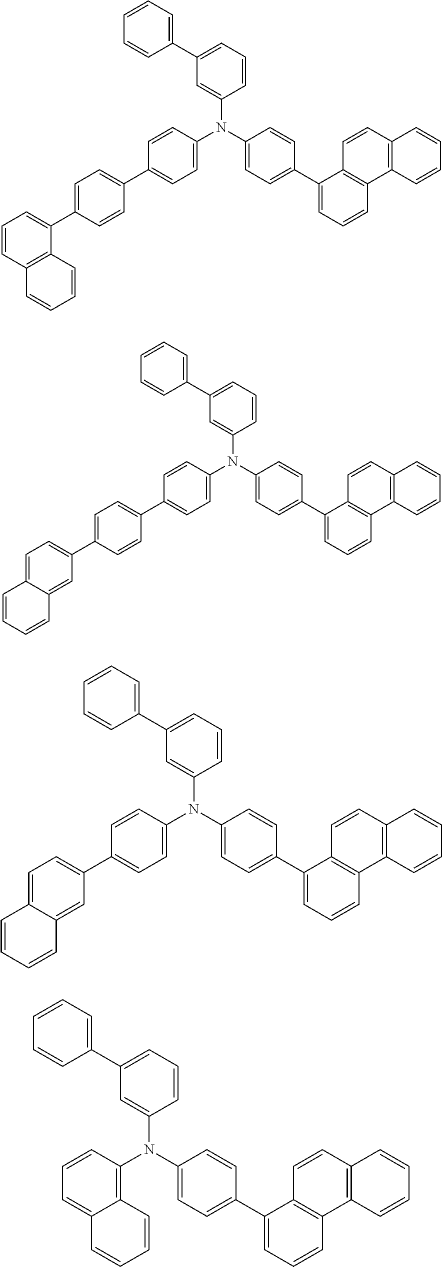 [SMILES: c1ccc(-c2cccc(N(c3ccc(-c4ccc(-c5cccc6ccccc56)cc4)cc3)c3ccc(-c4cccc5c4ccc4ccccc45)cc3)c2)cc1 ; c1ccc(-c2cccc(N(c3ccc(-c4ccc(-c5ccc6ccccc6c5)cc4)cc3)c3ccc(-c4cccc5c4ccc4ccccc45)cc3)c2)cc1 ; c1ccc(-c2cccc(N(c3ccc(-c4ccc5ccccc5c4)cc3)c3ccc(-c4cccc5c4ccc4ccccc45)cc3)c2)cc1 ; c1ccc(-c2cccc(N(c3ccc(-c4cccc5c4ccc4ccccc45)cc3)c3cccc4ccccc34)c2)cc1]

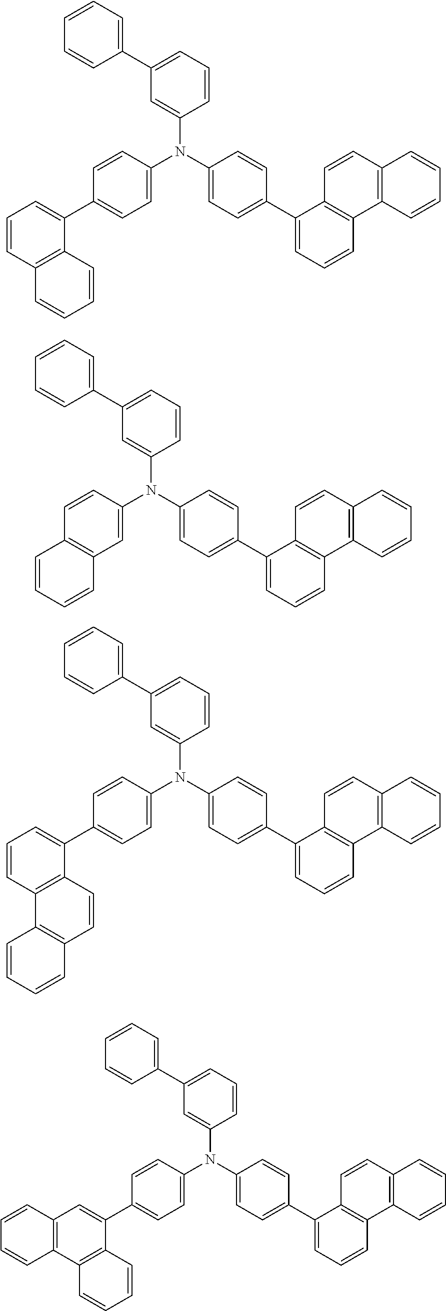

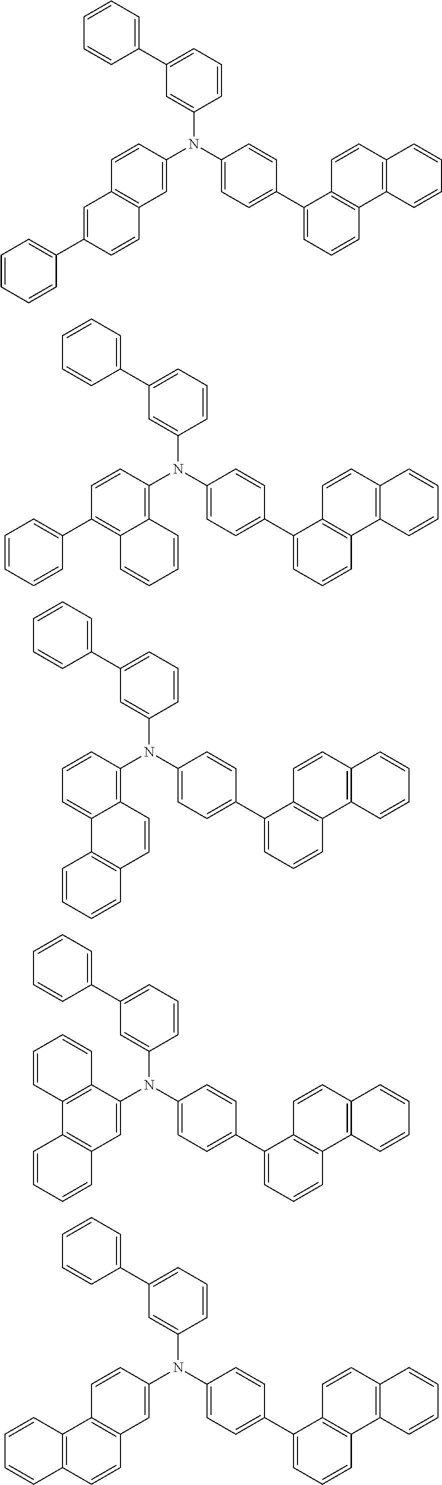

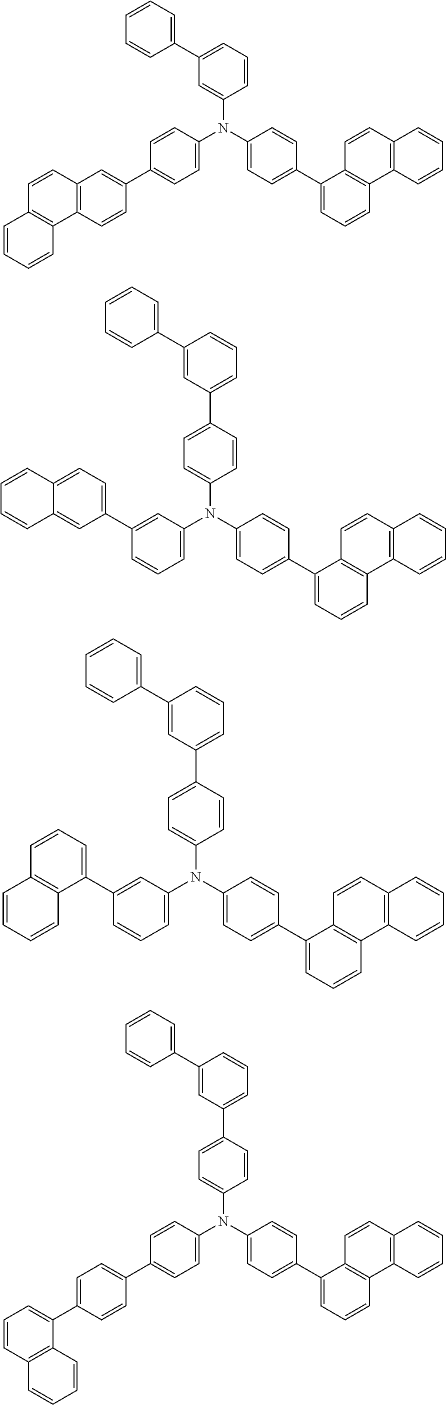 [SMILES: c1ccc(-c2cccc(N(c3ccc(-c4ccc5c(ccc6ccccc65)c4)cc3)c3ccc(-c4cccc5c4ccc4ccccc45)cc3)c2)cc1 ; c1ccc(-c2cccc(-c3ccc(N(c4ccc(-c5cccc6c5ccc5ccccc56)cc4)c4cccc(-c5ccc6ccccc6c5)c4)cc3)c2)cc1 ; c1ccc(-c2cccc(-c3ccc(N(c4ccc(-c5cccc6c5ccc5ccccc56)cc4)c4cccc(-c5cccc6ccccc56)c4)cc3)c2)cc1 ; c1ccc(-c2cccc(-c3ccc(N(c4ccc(-c5ccc(-c6cccc7ccccc67)cc5)cc4)c4ccc(-c5cccc6c5ccc5ccccc56)cc4)cc3)c2)cc1]

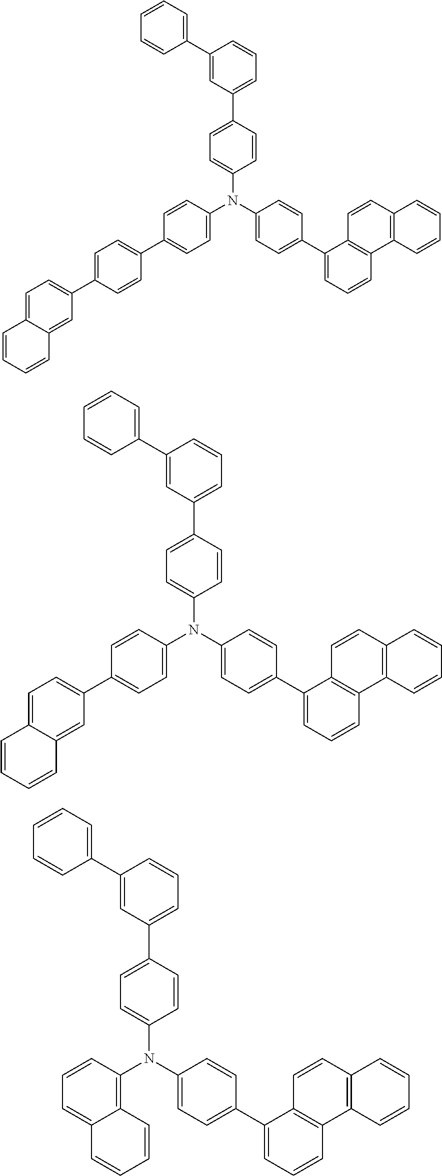

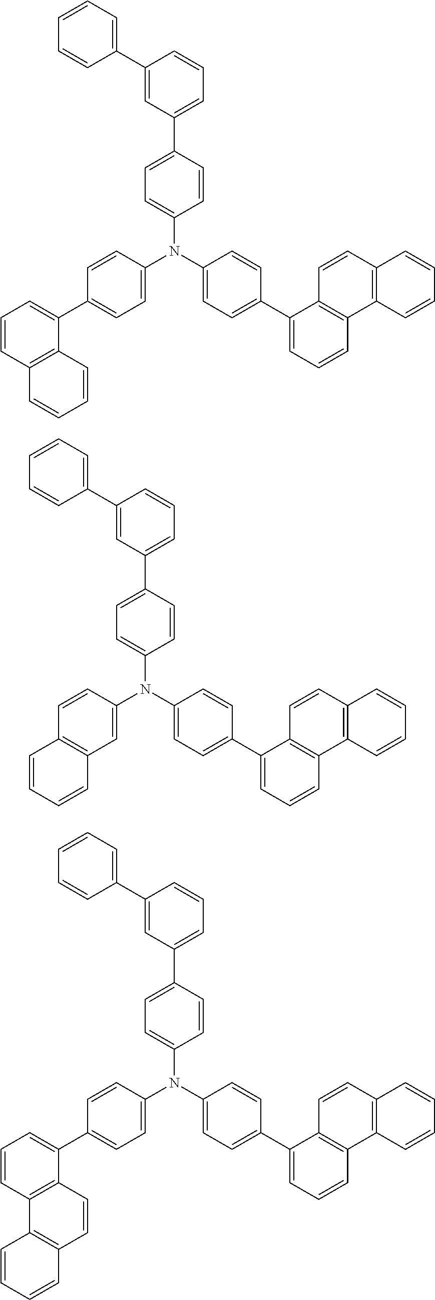

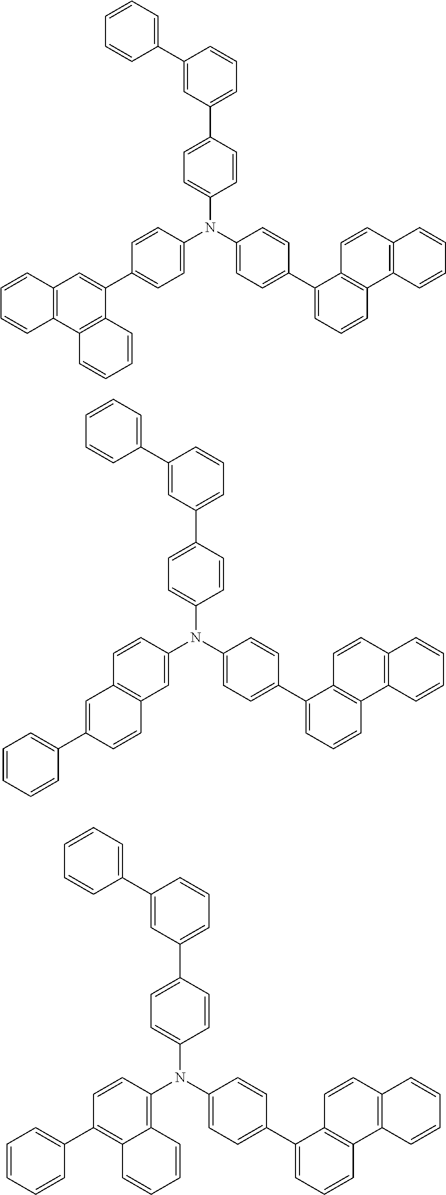

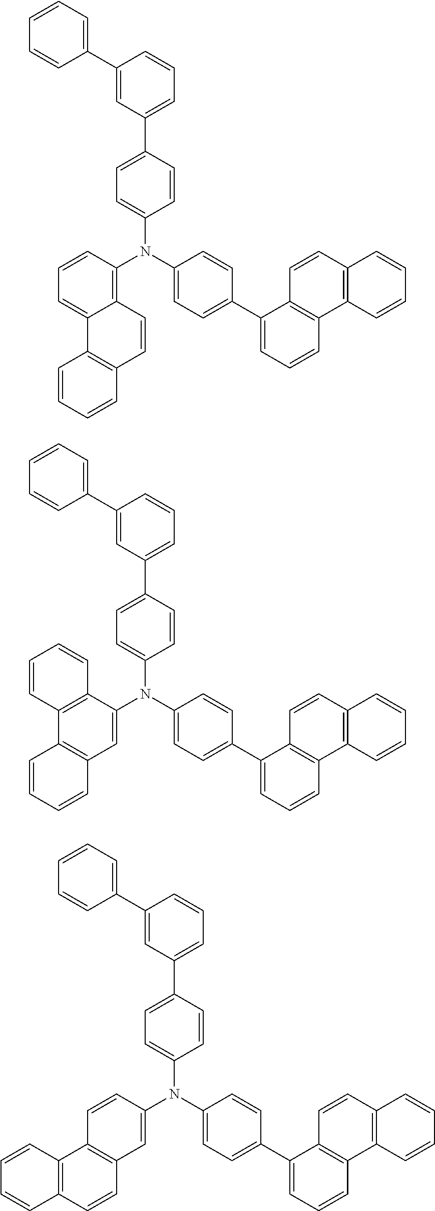

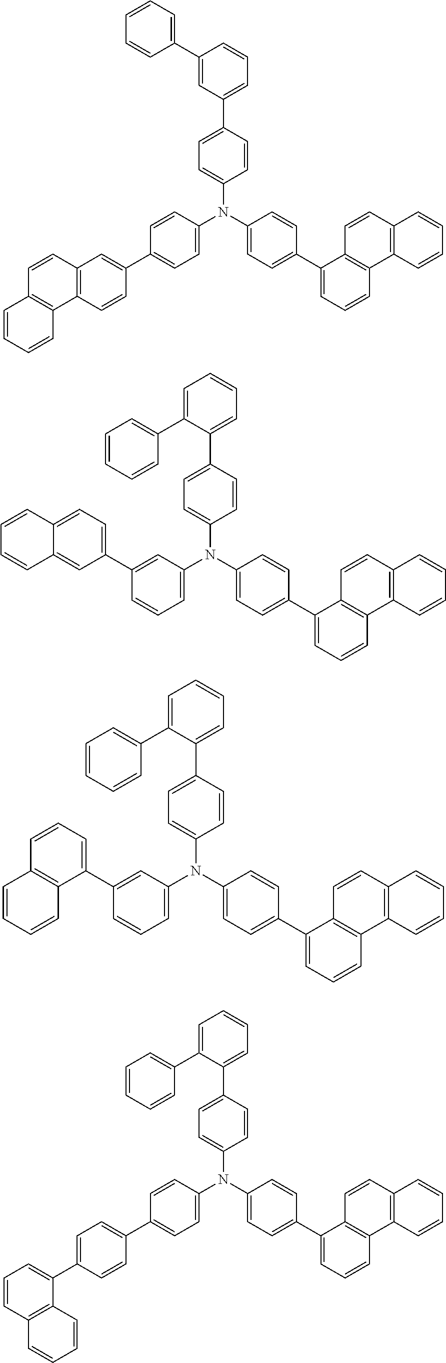 [SMILES: c1ccc(-c2cccc(-c3ccc(N(c4ccc(-c5ccc6c(ccc7ccccc76)c5)cc4)c4ccc(-c5cccc6c5ccc5ccccc56)cc4)cc3)c2)cc1 ; c1ccc(-c2ccccc2-c2ccc(N(c3ccc(-c4cccc5c4ccc4ccccc45)cc3)c3cccc(-c4ccc5ccccc5c4)c3)cc2)cc1 ; c1ccc(-c2ccccc2-c2ccc(N(c3ccc(-c4cccc5c4ccc4ccccc45)cc3)c3cccc(-c4cccc5ccccc45)c3)cc2)cc1 ; c1ccc(-c2ccccc2-c2ccc(N(c3ccc(-c4ccc(-c5cccc6ccccc56)cc4)cc3)c3ccc(-c4cccc5c4ccc4ccccc45)cc3)cc2)cc1]

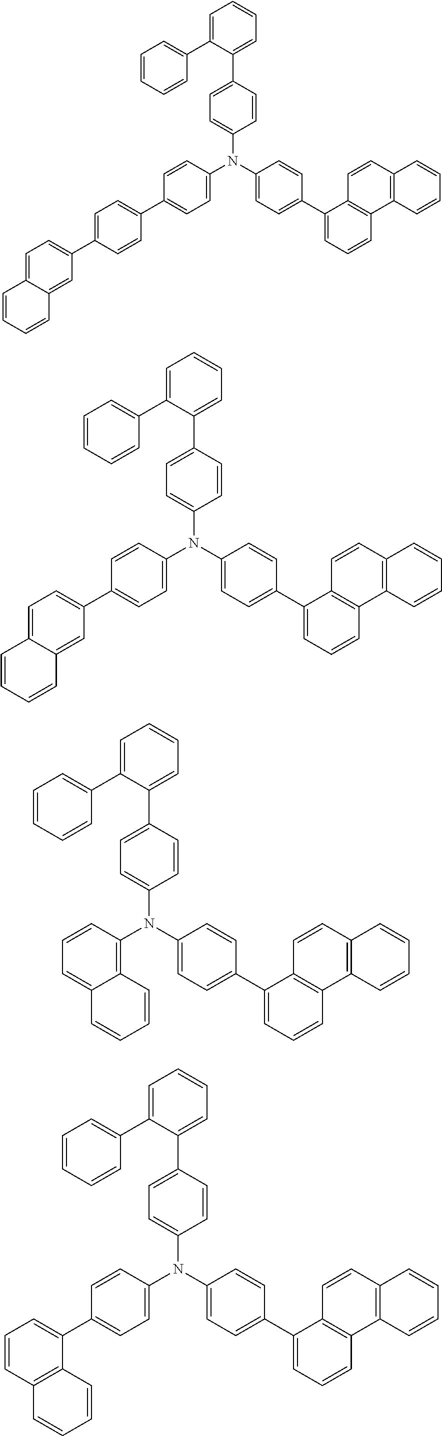 [SMILES: c1ccc(-c2ccccc2-c2ccc(N(c3ccc(-c4ccc(-c5ccc6ccccc6c5)cc4)cc3)c3ccc(-c4cccc5c4ccc4ccccc45)cc3)cc2)cc1 ; c1ccc(-c2ccccc2-c2ccc(N(c3ccc(-c4ccc5ccccc5c4)cc3)c3ccc(-c4cccc5c4ccc4ccccc45)cc3)cc2)cc1 ; c1ccc(-c2ccccc2-c2ccc(N(c3ccc(-c4cccc5c4ccc4ccccc45)cc3)c3cccc4ccccc34)cc2)cc1 ; c1ccc(-c2ccccc2-c2ccc(N(c3ccc(-c4cccc5ccccc45)cc3)c3ccc(-c4cccc5c4ccc4ccccc45)cc3)cc2)cc1]

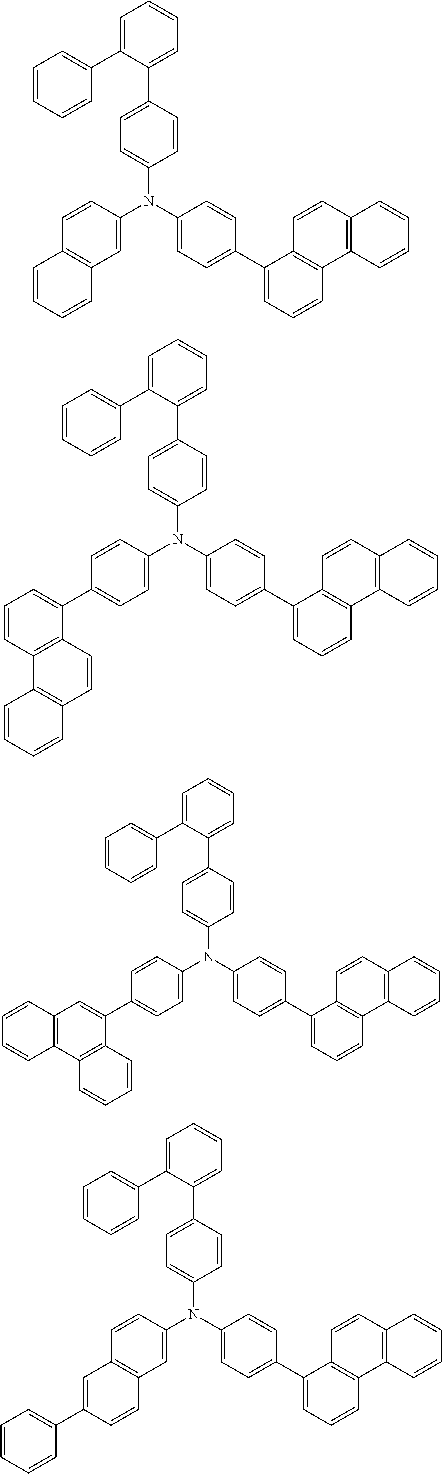 [SMILES: c1ccc(-c2ccccc2-c2ccc(N(c3ccc(-c4cccc5c4ccc4ccccc45)cc3)c3ccc4ccccc4c3)cc2)cc1 ; c1ccc(-c2ccccc2-c2ccc(N(c3ccc(-c4cccc5c4ccc4ccccc45)cc3)c3ccc(-c4cccc5c4ccc4ccccc45)cc3)cc2)cc1 ; c1ccc(-c2ccccc2-c2ccc(N(c3ccc(-c4cccc5c4ccc4ccccc45)cc3)c3ccc(-c4cc5ccccc5c5ccccc45)cc3)cc2)cc1 ; c1ccc(-c2ccc3cc(N(c4ccc(-c5ccccc5-c5ccccc5)cc4)c4ccc(-c5cccc6c5ccc5ccccc56)cc4)ccc3c2)cc1]

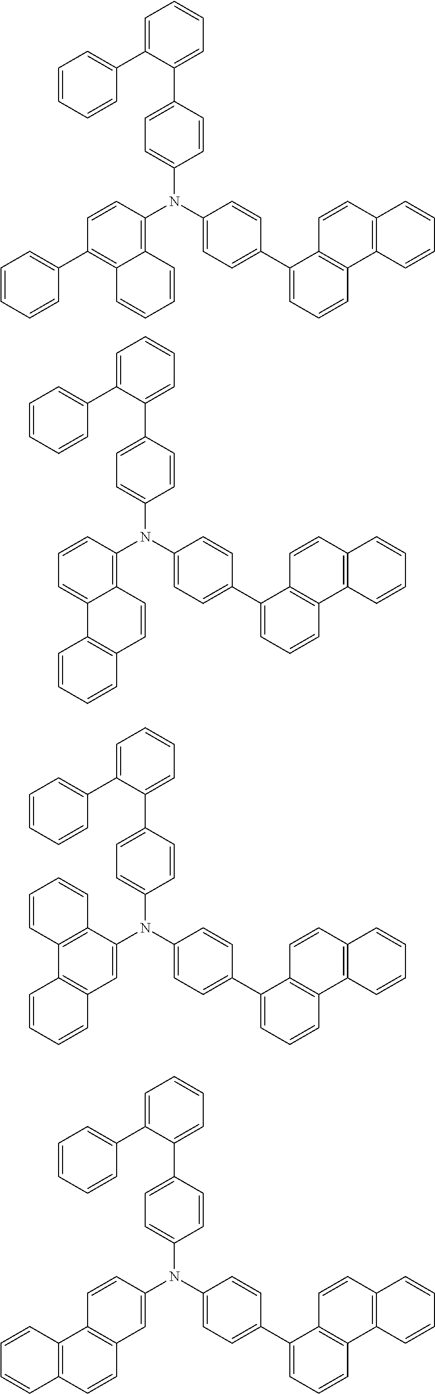

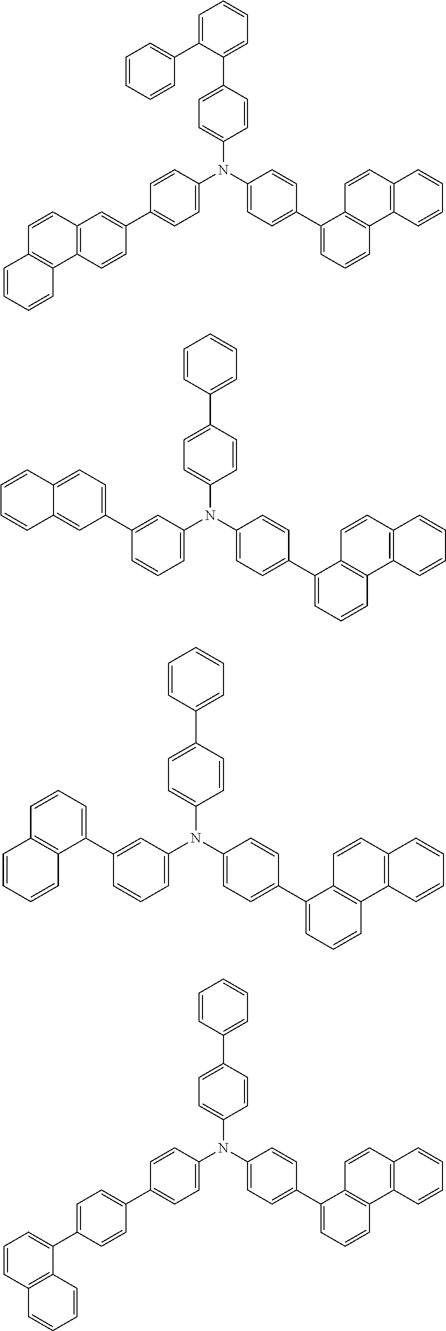 [SMILES: c1ccc(-c2ccccc2-c2ccc(N(c3ccc(-c4ccc5c(ccc6ccccc65)c4)cc3)c3ccc(-c4cccc5c4ccc4ccccc45)cc3)cc2)cc1 ; c1ccc(-c2ccc(N(c3ccc(-c4cccc5c4ccc4ccccc45)cc3)c3cccc(-c4ccc5ccccc5c4)c3)cc2)cc1 ; c1ccc(-c2ccc(N(c3ccc(-c4cccc5c4ccc4ccccc45)cc3)c3cccc(-c4cccc5ccccc45)c3)cc2)cc1 ; c1ccc(-c2ccc(N(c3ccc(-c4ccc(-c5cccc6ccccc56)cc4)cc3)c3ccc(-c4cccc5c4ccc4ccccc45)cc3)cc2)cc1]

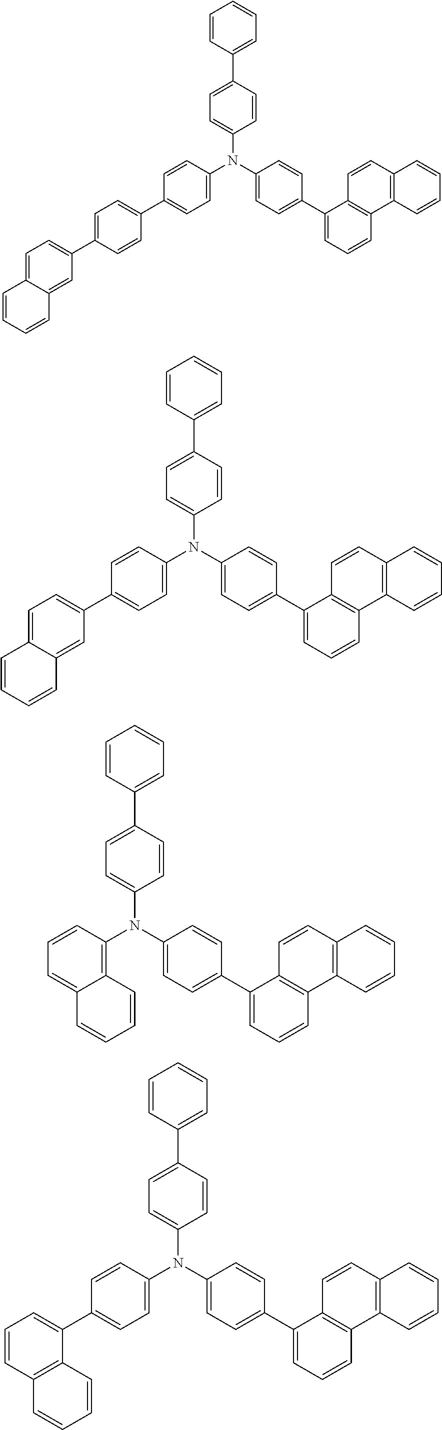 [SMILES: c1ccc(-c2ccc(N(c3ccc(-c4ccc(-c5ccc6ccccc6c5)cc4)cc3)c3ccc(-c4cccc5c4ccc4ccccc45)cc3)cc2)cc1 ; c1ccc(-c2ccc(N(c3ccc(-c4ccc5ccccc5c4)cc3)c3ccc(-c4cccc5c4ccc4ccccc45)cc3)cc2)cc1 ; c1ccc(-c2ccc(N(c3ccc(-c4cccc5c4ccc4ccccc45)cc3)c3cccc4ccccc34)cc2)cc1 ; c1ccc(-c2ccc(N(c3ccc(-c4cccc5ccccc45)cc3)c3ccc(-c4cccc5c4ccc4ccccc45)cc3)cc2)cc1]

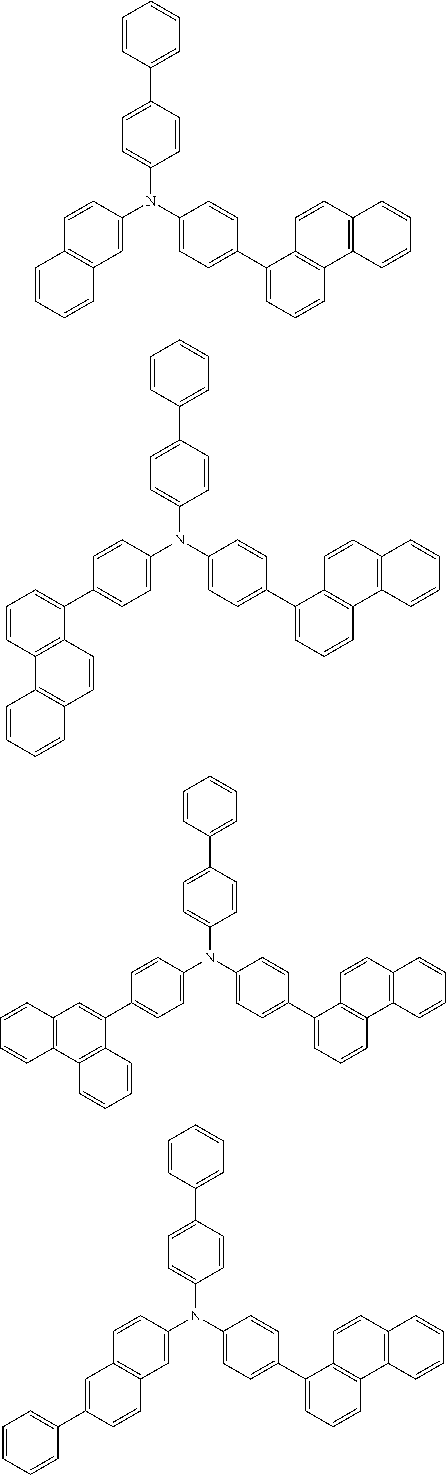 [SMILES: c1ccc(-c2ccc(N(c3ccc(-c4cccc5c4ccc4ccccc45)cc3)c3ccc4ccccc4c3)cc2)cc1 ; c1ccc(-c2ccc(N(c3ccc(-c4cccc5c4ccc4ccccc45)cc3)c3ccc(-c4cccc5c4ccc4ccccc45)cc3)cc2)cc1 ; c1ccc(-c2ccc(N(c3ccc(-c4cccc5c4ccc4ccccc45)cc3)c3ccc(-c4cc5ccccc5c5ccccc45)cc3)cc2)cc1 ; c1ccc(-c2ccc(N(c3ccc(-c4cccc5c4ccc4ccccc45)cc3)c3ccc4cc(-c5ccccc5)ccc4c3)cc2)cc1]

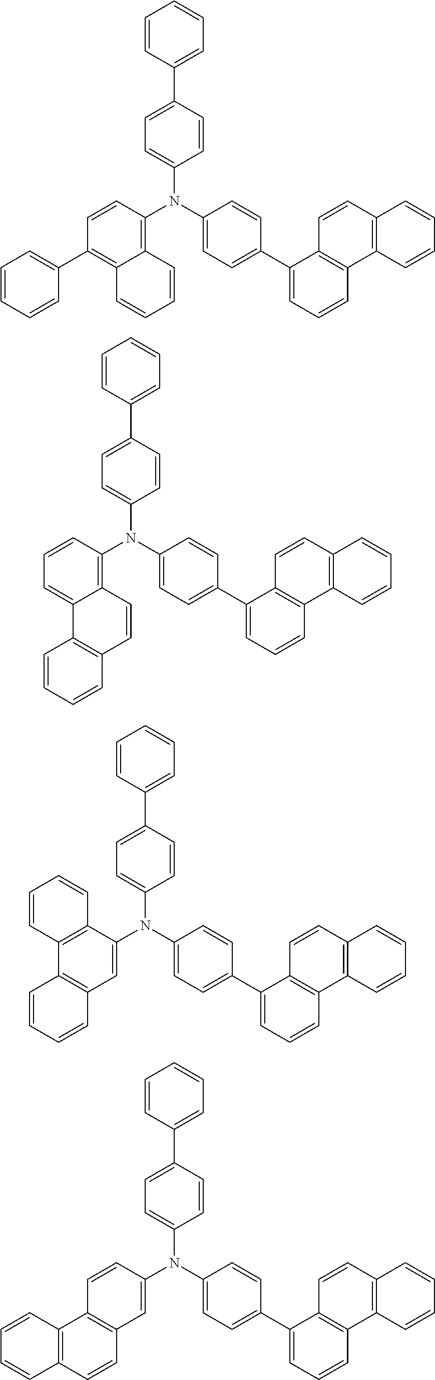

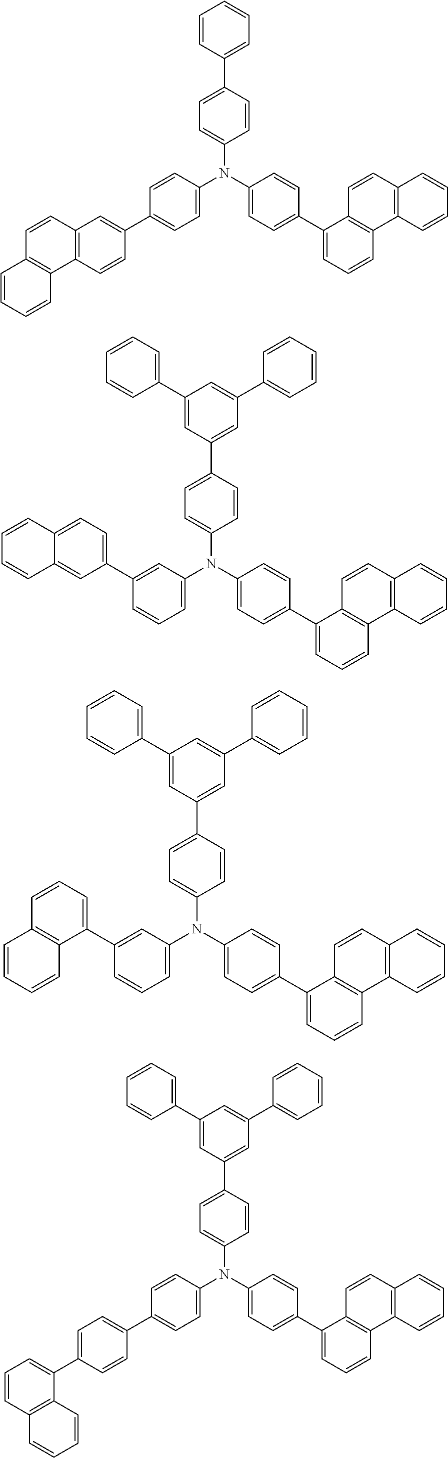

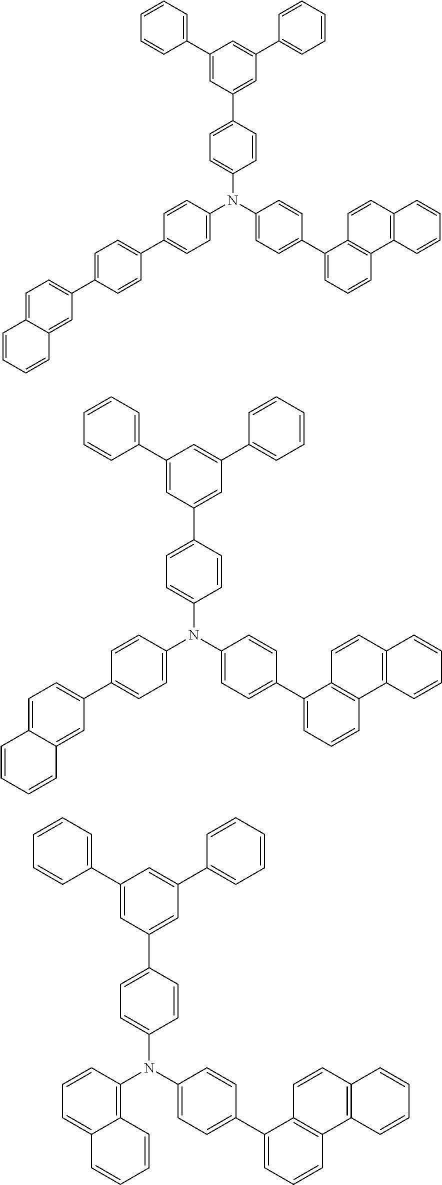

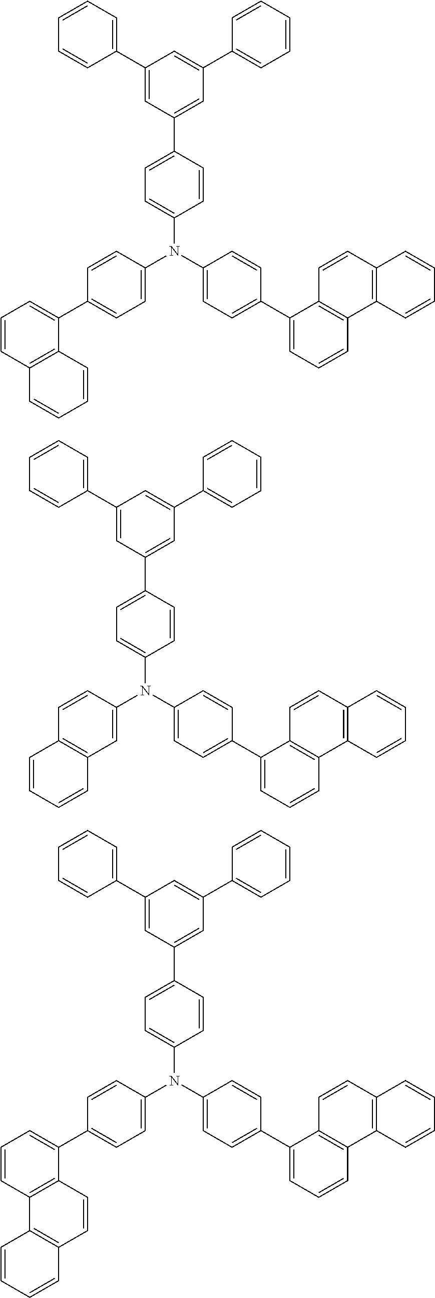

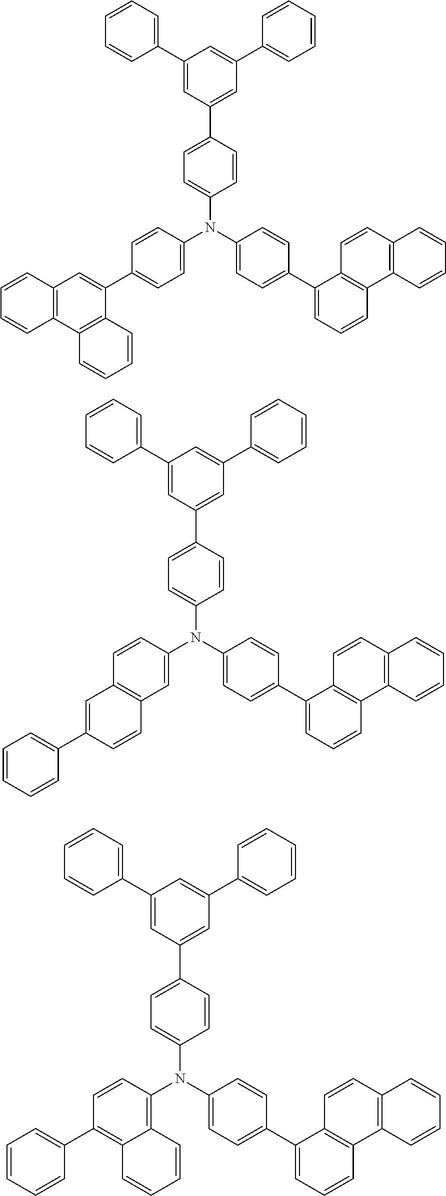

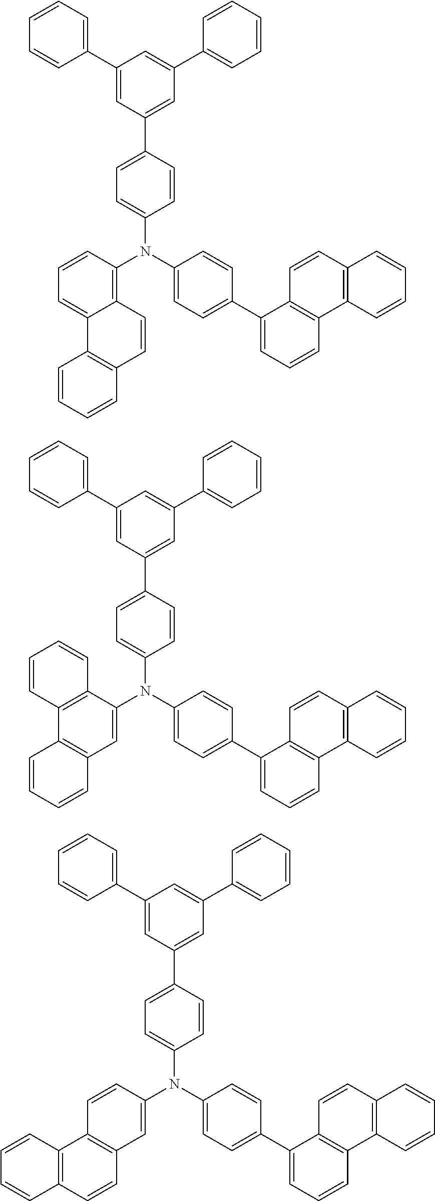

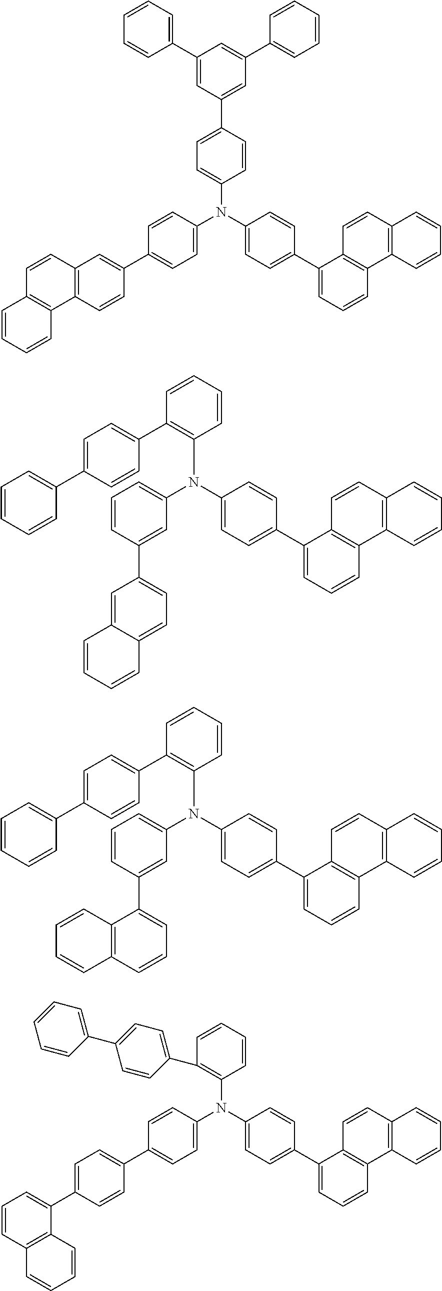

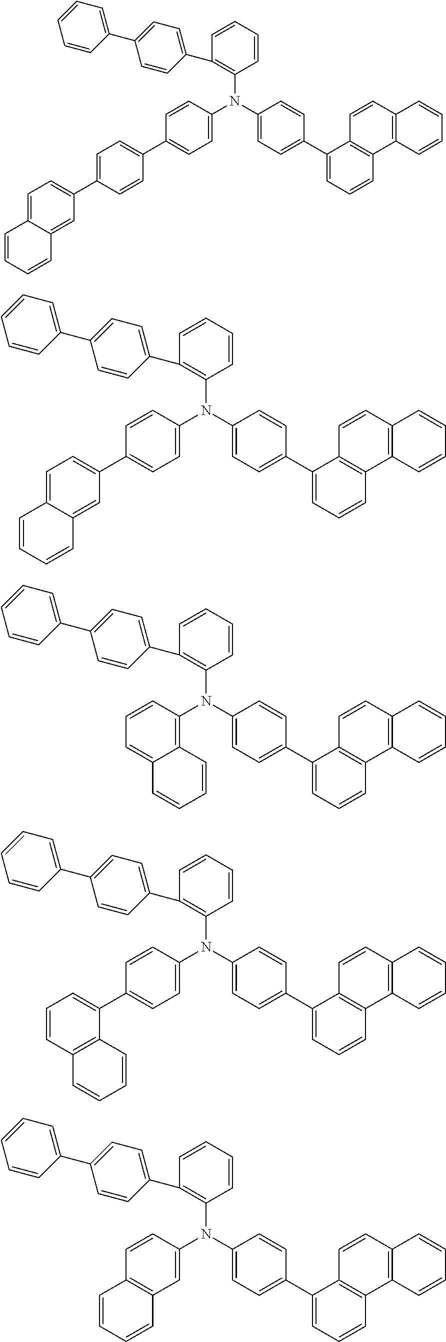

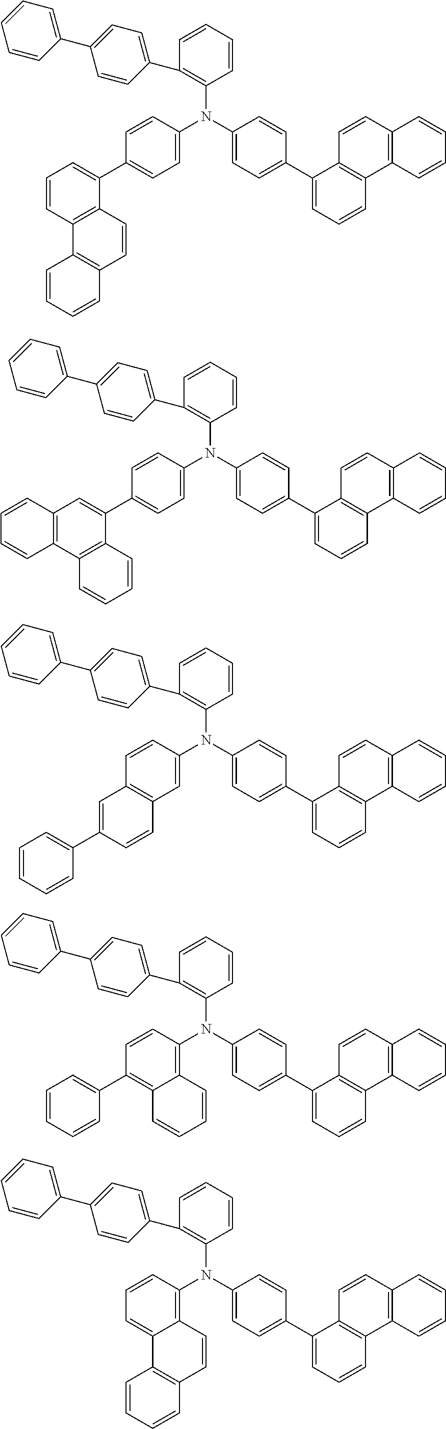

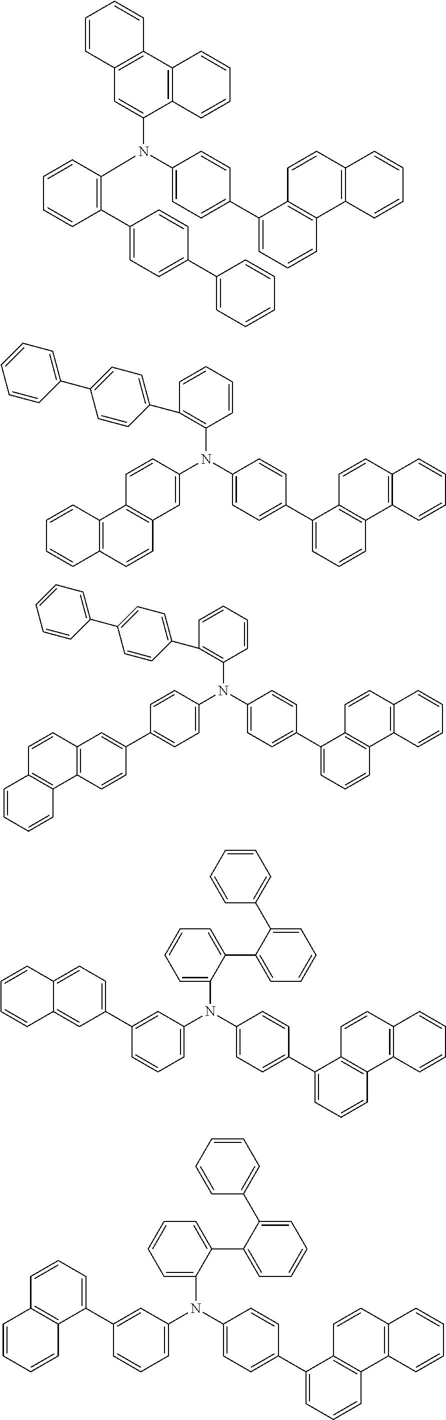

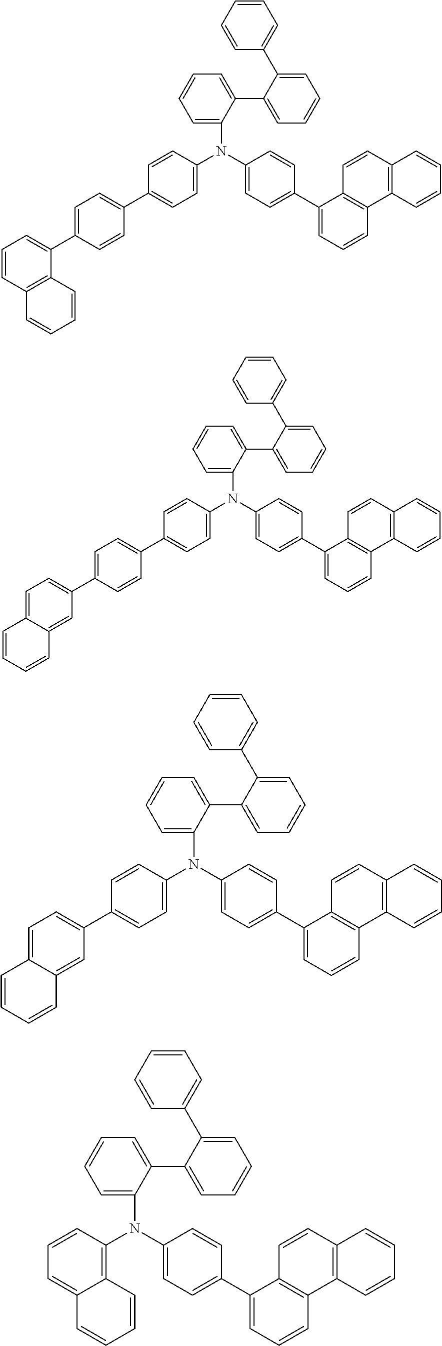 [SMILES: c1ccc(-c2ccccc2-c2ccccc2N(c2ccc(-c3ccc(-c4cccc5ccccc45)cc3)cc2)c2ccc(-c3cccc4c3ccc3ccccc34)cc2)cc1 ; c1ccc(-c2ccccc2-c2ccccc2N(c2ccc(-c3ccc(-c4ccc5ccccc5c4)cc3)cc2)c2ccc(-c3cccc4c3ccc3ccccc34)cc2)cc1 ; c1ccc(-c2ccccc2-c2ccccc2N(c2ccc(-c3ccc4ccccc4c3)cc2)c2ccc(-c3cccc4c3ccc3ccccc34)cc2)cc1 ; c1ccc(-c2ccccc2-c2ccccc2N(c2ccc(-c3cccc4c3ccc3ccccc34)cc2)c2cccc3ccccc23)cc1]

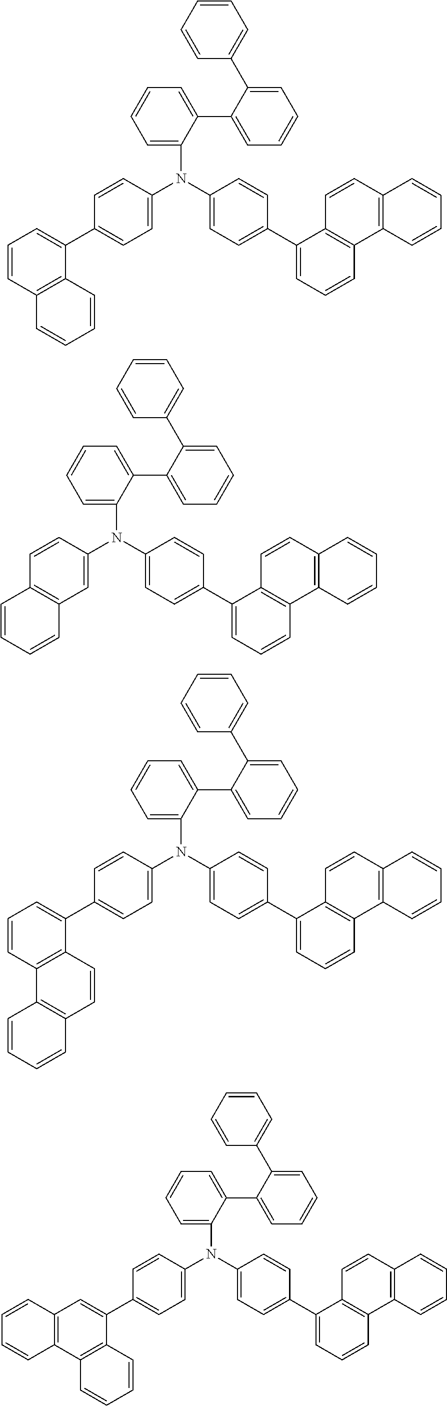

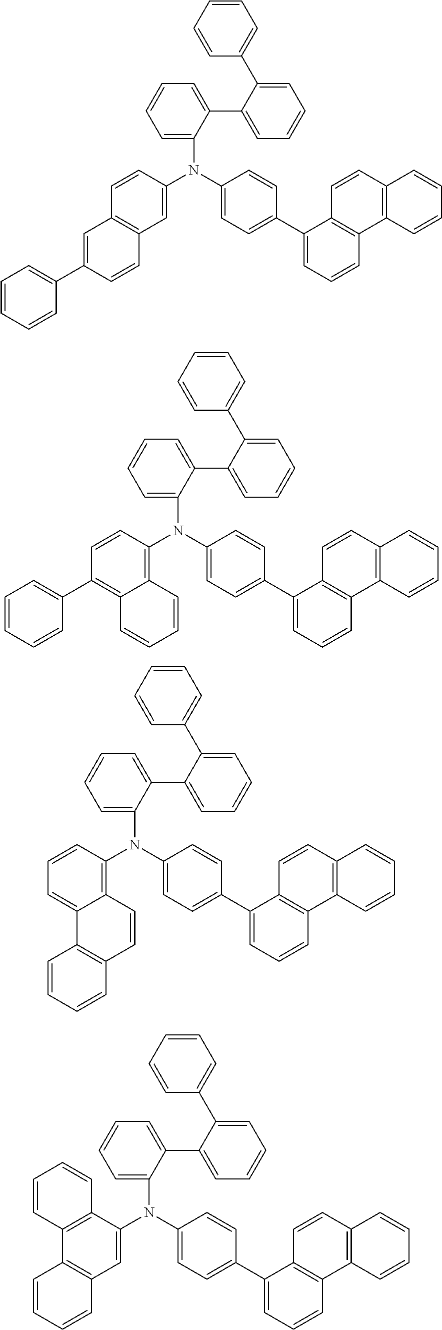

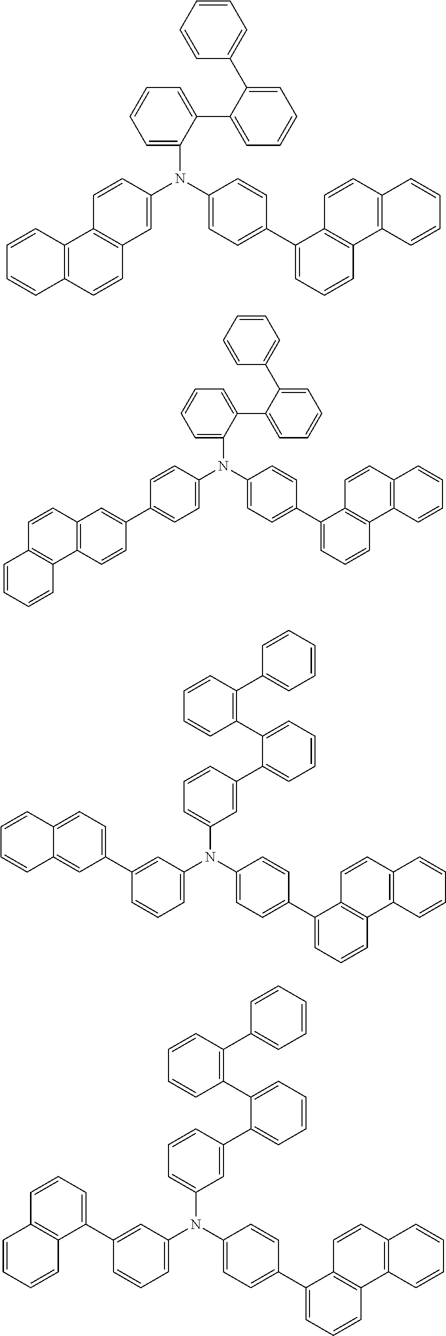 [SMILES: c1ccc(-c2ccccc2-c2ccccc2N(c2ccc(-c3cccc4c3ccc3ccccc34)cc2)c2ccc3c(ccc4ccccc43)c2)cc1 ; c1ccc(-c2ccccc2-c2ccccc2N(c2ccc(-c3ccc4c(ccc5ccccc54)c3)cc2)c2ccc(-c3cccc4c3ccc3ccccc34)cc2)cc1 ; c1ccc(-c2ccccc2-c2ccccc2-c2cccc(N(c3ccc(-c4cccc5c4ccc4ccccc45)cc3)c3cccc(-c4ccc5ccccc5c4)c3)c2)cc1 ; c1ccc(-c2ccccc2-c2ccccc2-c2cccc(N(c3ccc(-c4cccc5c4ccc4ccccc45)cc3)c3cccc(-c4cccc5ccccc45)c3)c2)cc1]

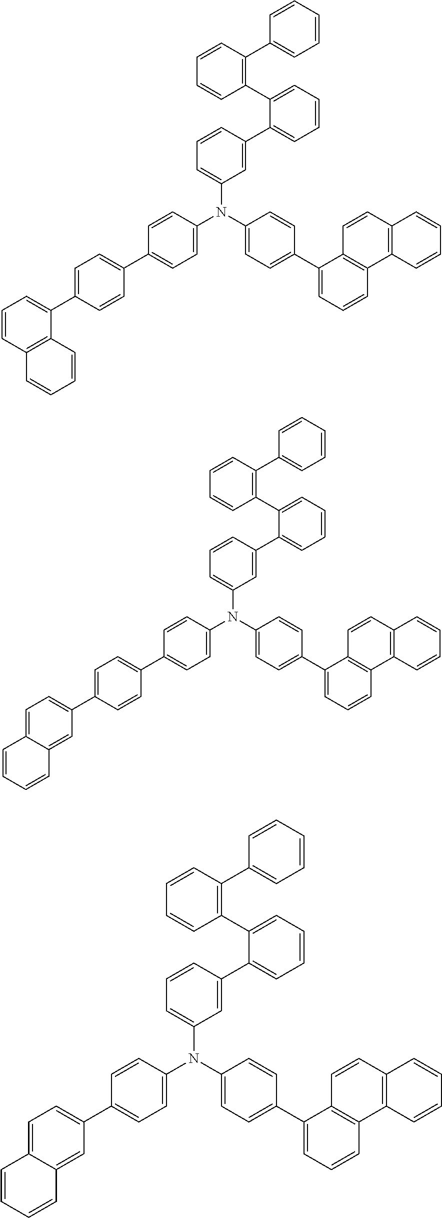 [SMILES: c1ccc(-c2ccccc2-c2ccccc2-c2cccc(N(c3ccc(-c4ccc(-c5cccc6ccccc56)cc4)cc3)c3ccc(-c4cccc5c4ccc4ccccc45)cc3)c2)cc1 ; c1ccc(-c2ccccc2-c2ccccc2-c2cccc(N(c3ccc(-c4ccc(-c5ccc6ccccc6c5)cc4)cc3)c3ccc(-c4cccc5c4ccc4ccccc45)cc3)c2)cc1 ; c1ccc(-c2ccccc2-c2ccccc2-c2cccc(N(c3ccc(-c4ccc5ccccc5c4)cc3)c3ccc(-c4cccc5c4ccc4ccccc45)cc3)c2)cc1]

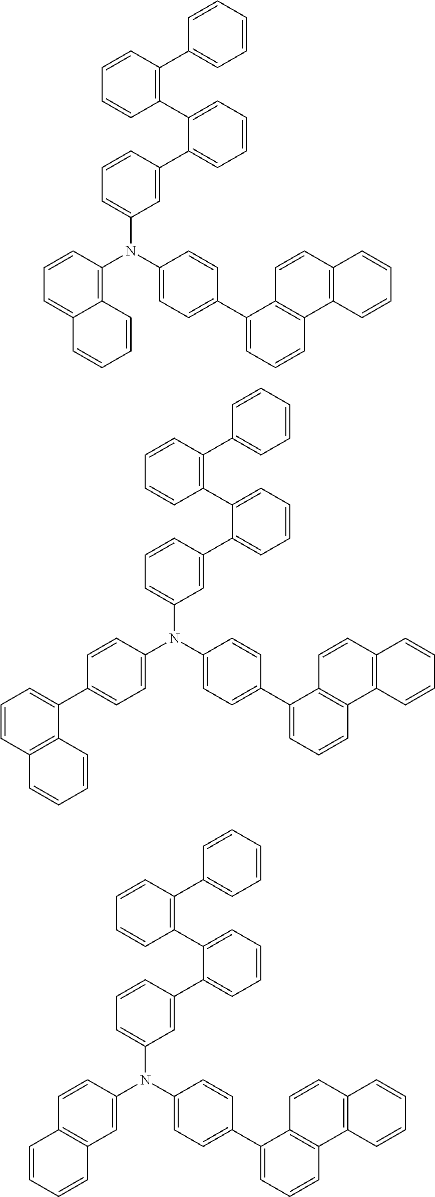

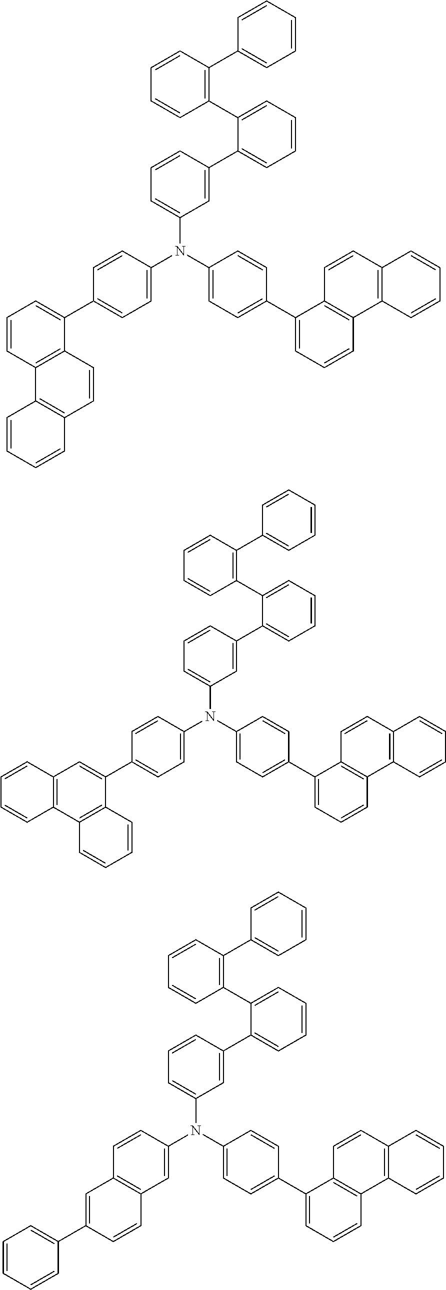 [SMILES: c1ccc(-c2ccccc2-c2ccccc2-c2cccc(N(c3ccc(-c4cccc5c4ccc4ccccc45)cc3)c3ccc(-c4cccc5c4ccc4ccccc45)cc3)c2)cc1 ; c1ccc(-c2ccccc2-c2ccccc2-c2cccc(N(c3ccc(-c4cccc5c4ccc4ccccc45)cc3)c3ccc(-c4cc5ccccc5c5ccccc45)cc3)c2)cc1 ; c1ccc(-c2ccc3cc(N(c4ccc(-c5cccc6c5ccc5ccccc56)cc4)c4cccc(-c5ccccc5-c5ccccc5-c5ccccc5)c4)ccc3c2)cc1]

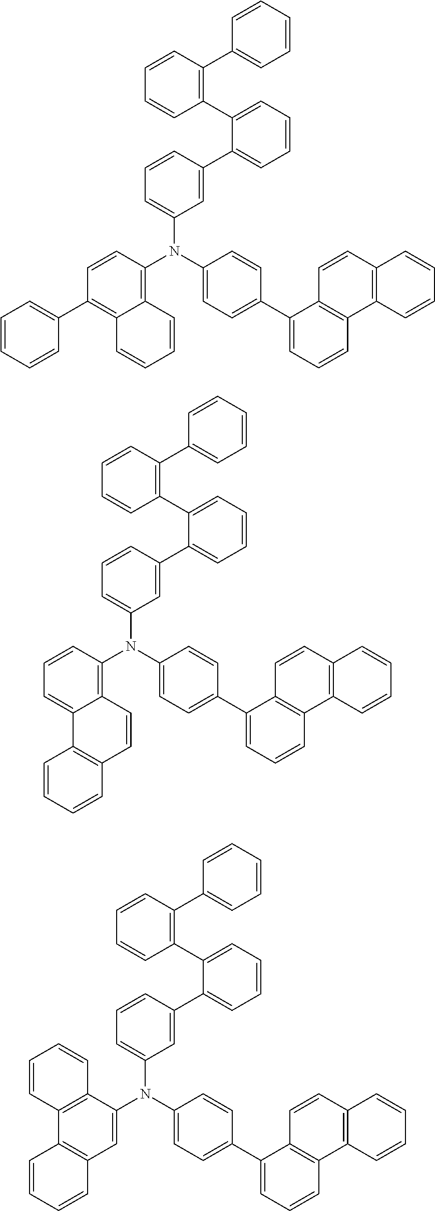 [SMILES: c1ccc(-c2ccccc2-c2ccccc2-c2cccc(N(c3ccc(-c4cccc5c4ccc4ccccc45)cc3)c3ccc(-c4ccccc4)c4ccccc34)c2)cc1 ; c1ccc(-c2ccccc2-c2ccccc2-c2cccc(N(c3ccc(-c4cccc5c4ccc4ccccc45)cc3)c3cccc4c3ccc3ccccc34)c2)cc1 ; c1ccc(-c2ccccc2-c2ccccc2-c2cccc(N(c3ccc(-c4cccc5c4ccc4ccccc45)cc3)c3cc4ccccc4c4ccccc34)c2)cc1]

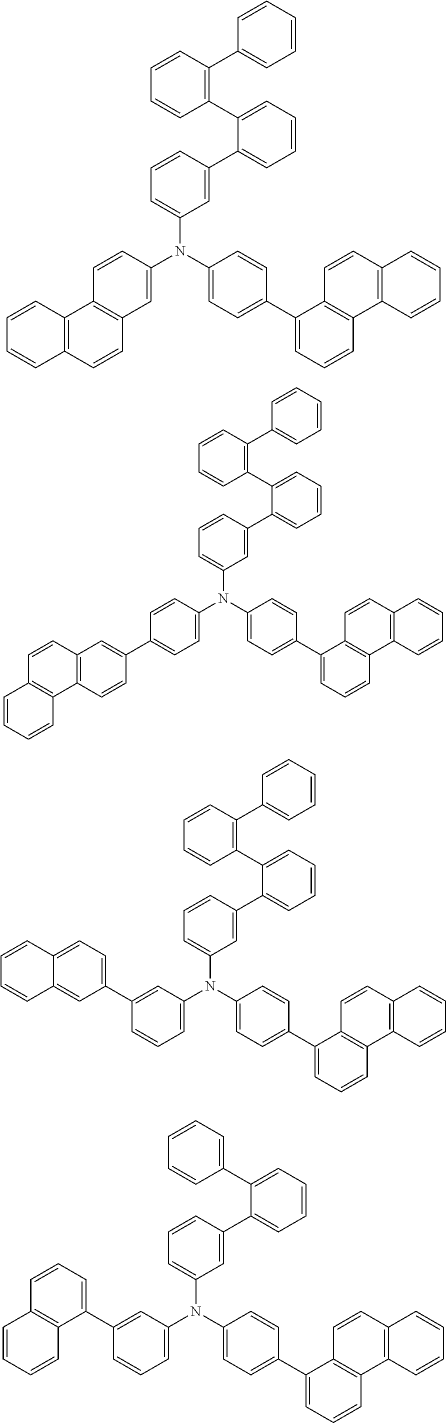

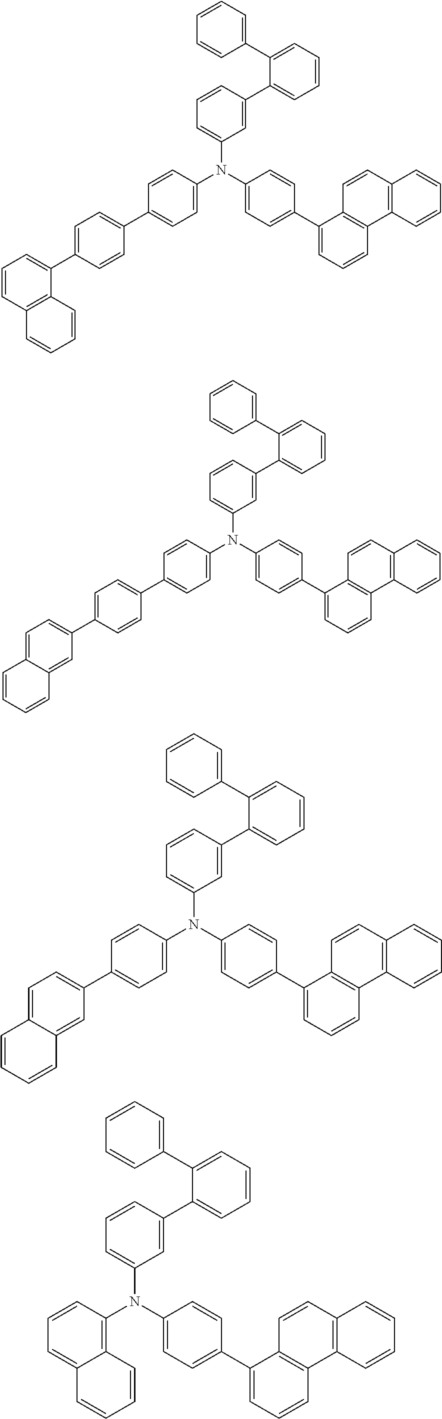 [SMILES: c1ccc(-c2ccccc2-c2cccc(N(c3ccc(-c4ccc(-c5cccc6ccccc56)cc4)cc3)c3ccc(-c4cccc5c4ccc4ccccc45)cc3)c2)cc1 ; c1ccc(-c2ccccc2-c2cccc(N(c3ccc(-c4ccc(-c5ccc6ccccc6c5)cc4)cc3)c3ccc(-c4cccc5c4ccc4ccccc45)cc3)c2)cc1 ; c1ccc(-c2ccccc2-c2cccc(N(c3ccc(-c4ccc5ccccc5c4)cc3)c3ccc(-c4cccc5c4ccc4ccccc45)cc3)c2)cc1 ; c1ccc(-c2ccccc2-c2cccc(N(c3ccc(-c4cccc5c4ccc4ccccc45)cc3)c3cccc4ccccc34)c2)cc1]

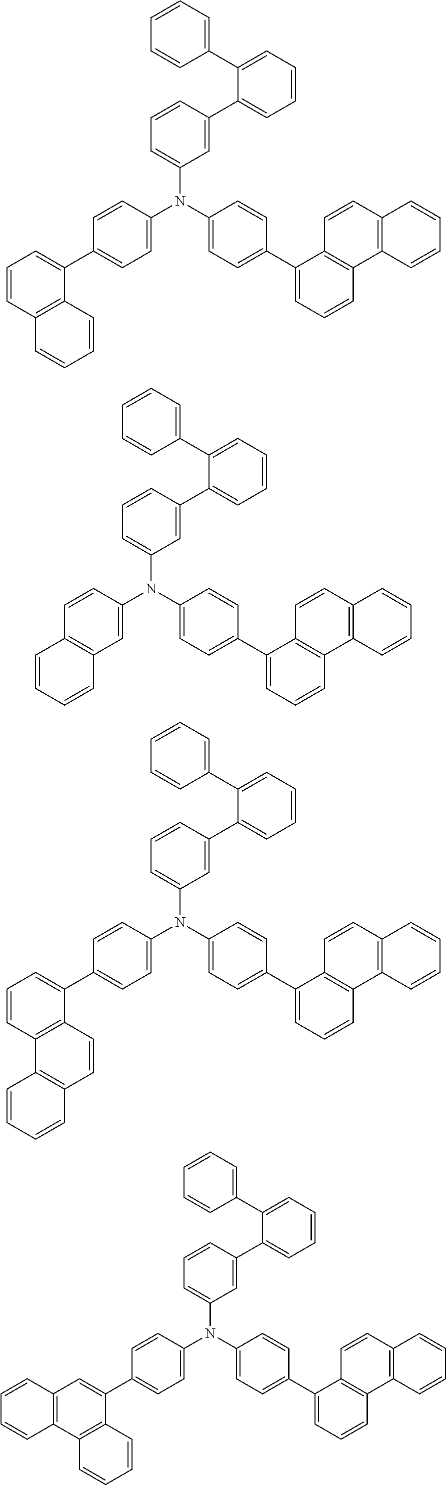 [SMILES: c1ccc(-c2ccccc2-c2cccc(N(c3ccc(-c4cccc5ccccc45)cc3)c3ccc(-c4cccc5c4ccc4ccccc45)cc3)c2)cc1 ; c1ccc(-c2ccccc2-c2cccc(N(c3ccc(-c4cccc5c4ccc4ccccc45)cc3)c3ccc4ccccc4c3)c2)cc1 ; c1ccc(-c2ccccc2-c2cccc(N(c3ccc(-c4cccc5c4ccc4ccccc45)cc3)c3ccc(-c4cccc5c4ccc4ccccc45)cc3)c2)cc1 ; c1ccc(-c2ccccc2-c2cccc(N(c3ccc(-c4cccc5c4ccc4ccccc45)cc3)c3ccc(-c4cc5ccccc5c5ccccc45)cc3)c2)cc1]

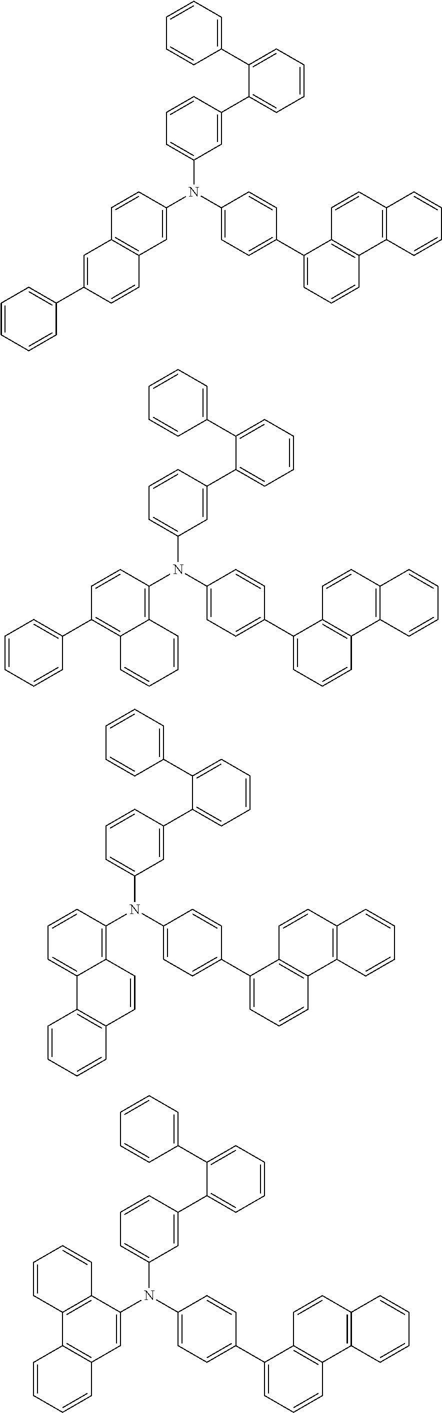

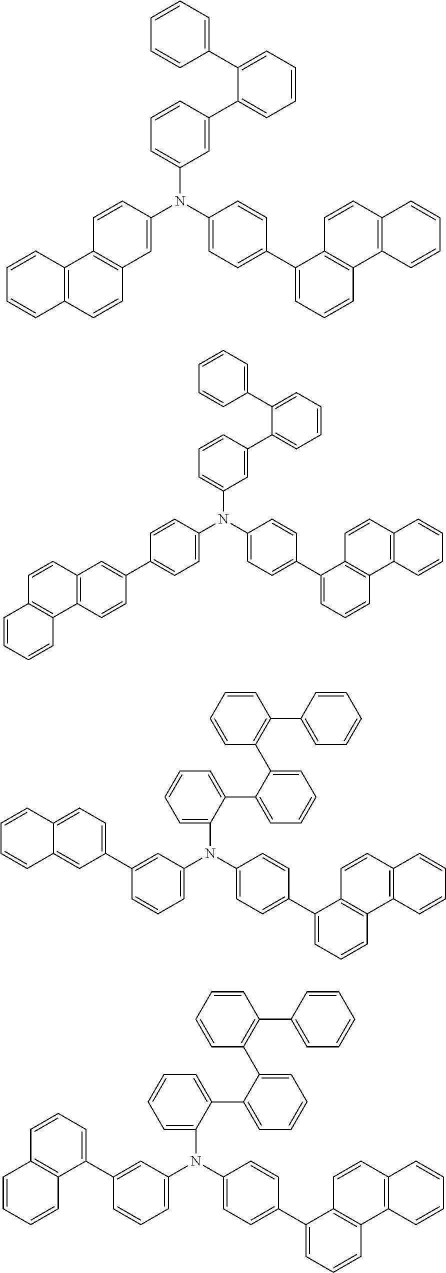

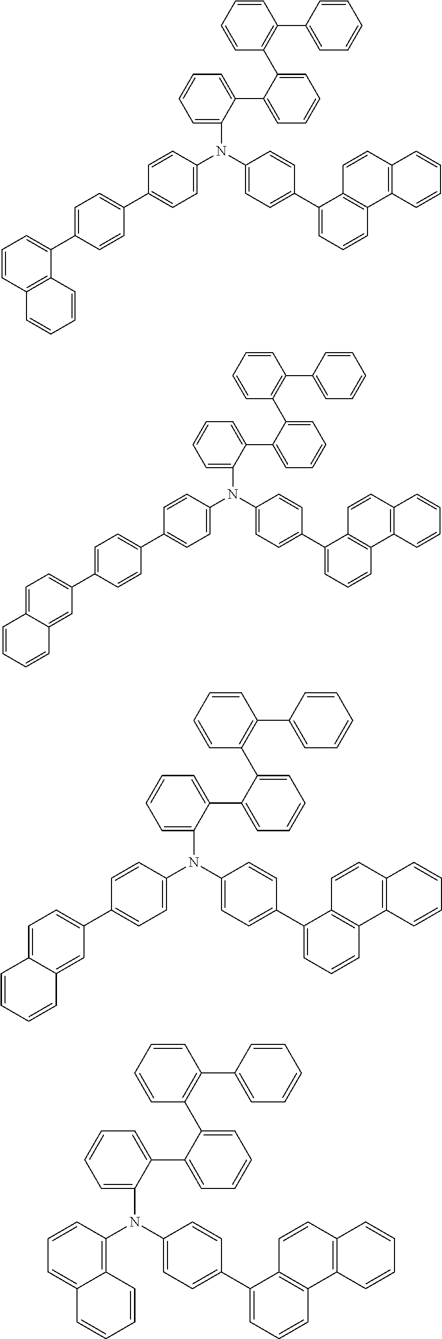 [SMILES: c1ccc(-c2ccccc2-c2ccccc2-c2ccccc2N(c2ccc(-c3ccc(-c4cccc5ccccc45)cc3)cc2)c2ccc(-c3cccc4c3ccc3ccccc34)cc2)cc1 ; c1ccc(-c2ccccc2-c2ccccc2-c2ccccc2N(c2ccc(-c3ccc(-c4ccc5ccccc5c4)cc3)cc2)c2ccc(-c3cccc4c3ccc3ccccc34)cc2)cc1 ; c1ccc(-c2ccccc2-c2ccccc2-c2ccccc2N(c2ccc(-c3ccc4ccccc4c3)cc2)c2ccc(-c3cccc4c3ccc3ccccc34)cc2)cc1 ; c1ccc(-c2ccccc2-c2ccccc2-c2ccccc2N(c2ccc(-c3cccc4c3ccc3ccccc34)cc2)c2cccc3ccccc23)cc1]

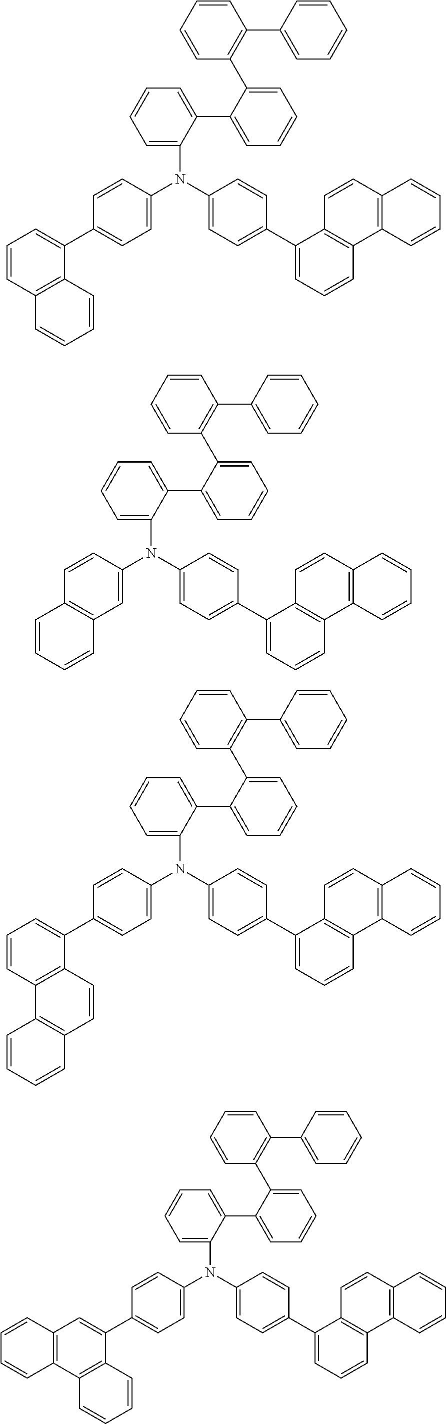

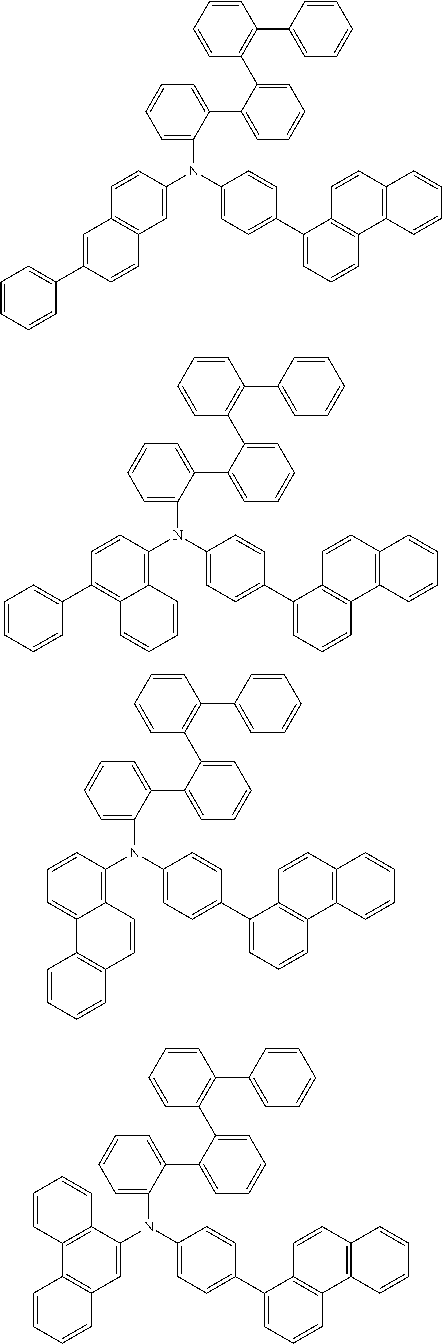

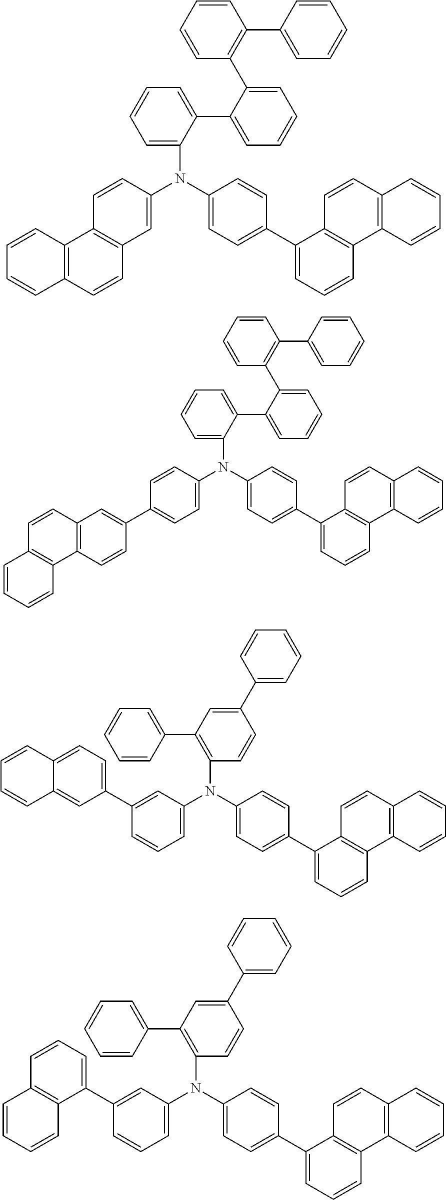

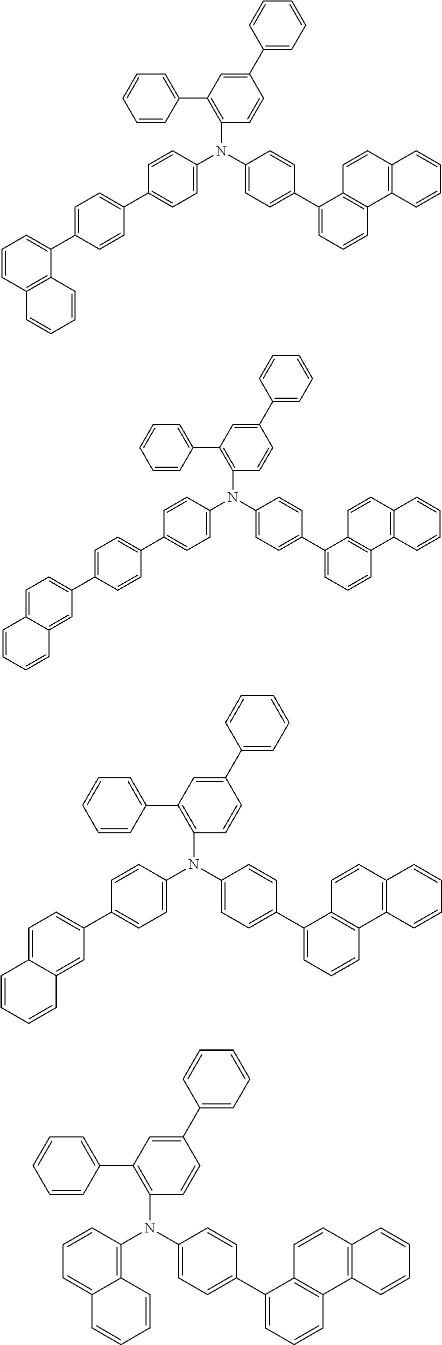 [SMILES: c1ccc(-c2ccc(N(c3ccc(-c4ccc(-c5cccc6ccccc56)cc4)cc3)c3ccc(-c4cccc5c4ccc4ccccc45)cc3)c(-c3ccccc3)c2)cc1 ; c1ccc(-c2ccc(N(c3ccc(-c4ccc(-c5ccc6ccccc6c5)cc4)cc3)c3ccc(-c4cccc5c4ccc4ccccc45)cc3)c(-c3ccccc3)c2)cc1 ; c1ccc(-c2ccc(N(c3ccc(-c4ccc5ccccc5c4)cc3)c3ccc(-c4cccc5c4ccc4ccccc45)cc3)c(-c3ccccc3)c2)cc1 ; c1ccc(-c2ccc(N(c3ccc(-c4cccc5c4ccc4ccccc45)cc3)c3cccc4ccccc34)c(-c3ccccc3)c2)cc1]

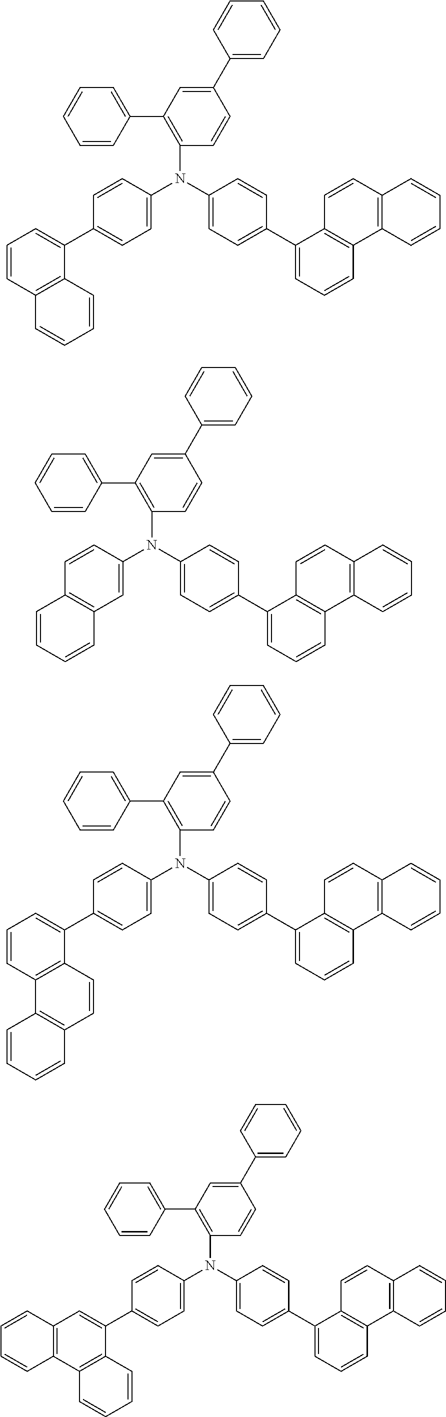

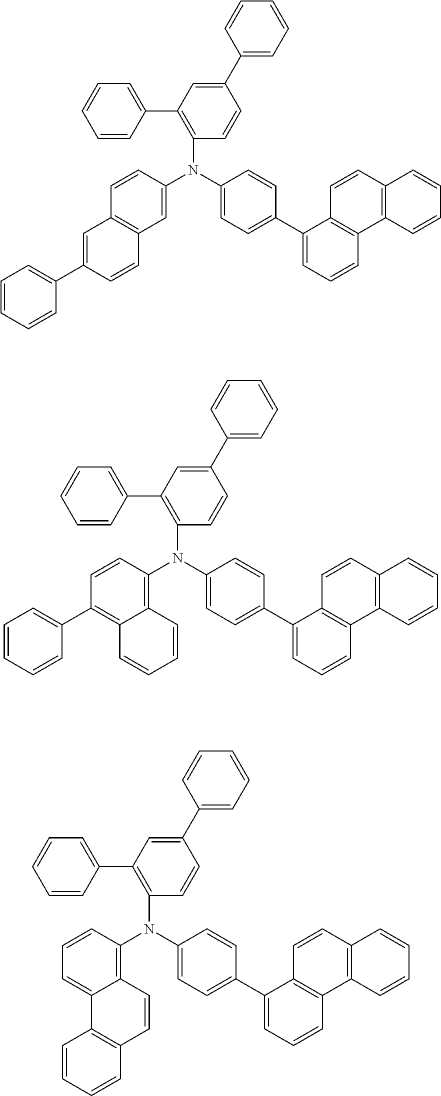 [SMILES: c1ccc(-c2ccc(N(c3ccc(-c4cccc5c4ccc4ccccc45)cc3)c3ccc4cc(-c5ccccc5)ccc4c3)c(-c3ccccc3)c2)cc1 ; c1ccc(-c2ccc(N(c3ccc(-c4cccc5c4ccc4ccccc45)cc3)c3ccc(-c4ccccc4)c4ccccc34)c(-c3ccccc3)c2)cc1 ; c1ccc(-c2ccc(N(c3ccc(-c4cccc5c4ccc4ccccc45)cc3)c3cccc4c3ccc3ccccc34)c(-c3ccccc3)c2)cc1]

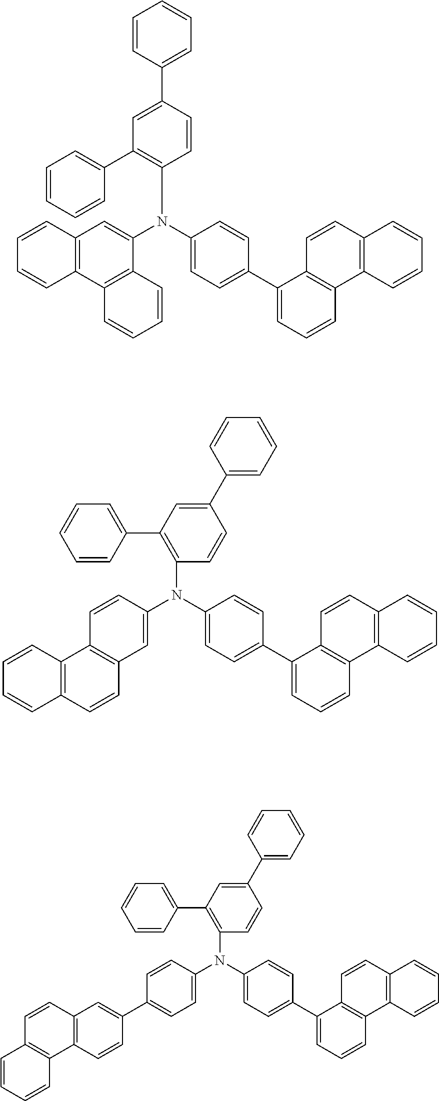 [SMILES: c1ccc(-c2ccc(N(c3ccc(-c4cccc5c4ccc4ccccc45)cc3)c3cc4ccccc4c4ccccc34)c(-c3ccccc3)c2)cc1 ; c1ccc(-c2ccc(N(c3ccc(-c4cccc5c4ccc4ccccc45)cc3)c3ccc4c(ccc5ccccc54)c3)c(-c3ccccc3)c2)cc1 ; c1ccc(-c2ccc(N(c3ccc(-c4ccc5c(ccc6ccccc65)c4)cc3)c3ccc(-c4cccc5c4ccc4ccccc45)cc3)c(-c3ccccc3)c2)cc1]

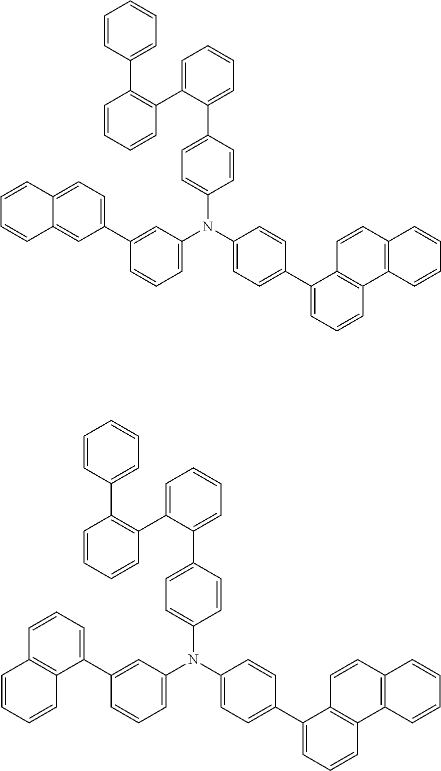 [SMILES: c1ccc(-c2ccccc2-c2ccccc2-c2ccc(N(c3ccc(-c4cccc5c4ccc4ccccc45)cc3)c3cccc(-c4ccc5ccccc5c4)c3)cc2)cc1 ; c1ccc(-c2ccccc2-c2ccccc2-c2ccc(N(c3ccc(-c4cccc5c4ccc4ccccc45)cc3)c3cccc(-c4cccc5ccccc45)c3)cc2)cc1]

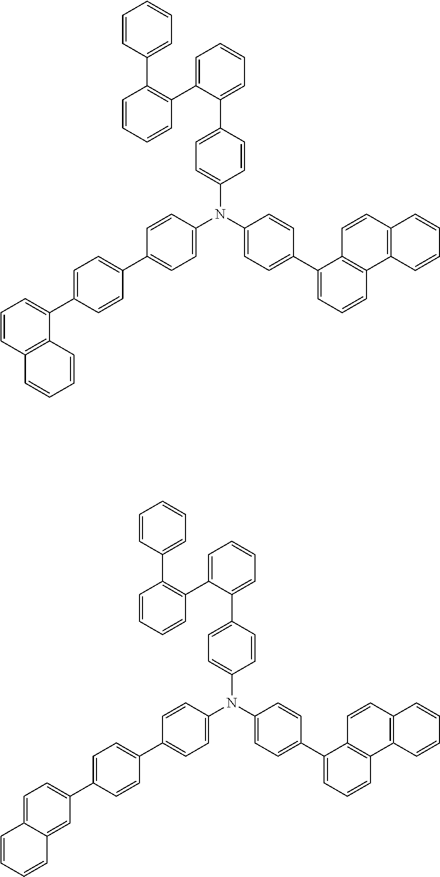 [SMILES: c1ccc(-c2ccccc2-c2ccccc2-c2ccc(N(c3ccc(-c4ccc(-c5cccc6ccccc56)cc4)cc3)c3ccc(-c4cccc5c4ccc4ccccc45)cc3)cc2)cc1 ; c1ccc(-c2ccccc2-c2ccccc2-c2ccc(N(c3ccc(-c4ccc(-c5ccc6ccccc6c5)cc4)cc3)c3ccc(-c4cccc5c4ccc4ccccc45)cc3)cc2)cc1]

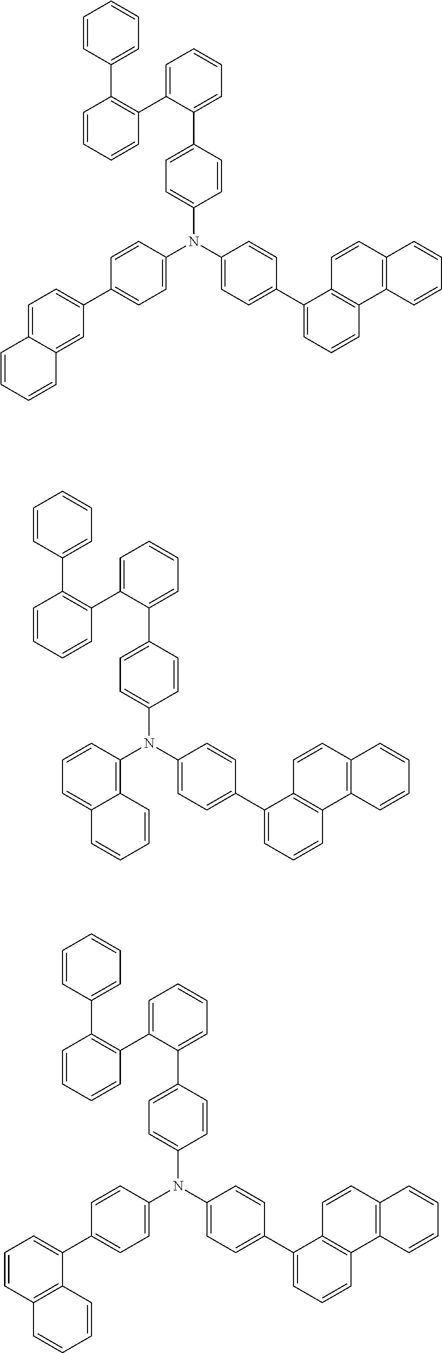 [SMILES: c1ccc(-c2ccccc2-c2ccccc2-c2ccc(N(c3ccc(-c4ccc5ccccc5c4)cc3)c3ccc(-c4cccc5c4ccc4ccccc45)cc3)cc2)cc1 ; c1ccc(-c2ccccc2-c2ccccc2-c2ccc(N(c3ccc(-c4cccc5c4ccc4ccccc45)cc3)c3cccc4ccccc34)cc2)cc1 ; c1ccc(-c2ccccc2-c2ccccc2-c2ccc(N(c3ccc(-c4cccc5ccccc45)cc3)c3ccc(-c4cccc5c4ccc4ccccc45)cc3)cc2)cc1]

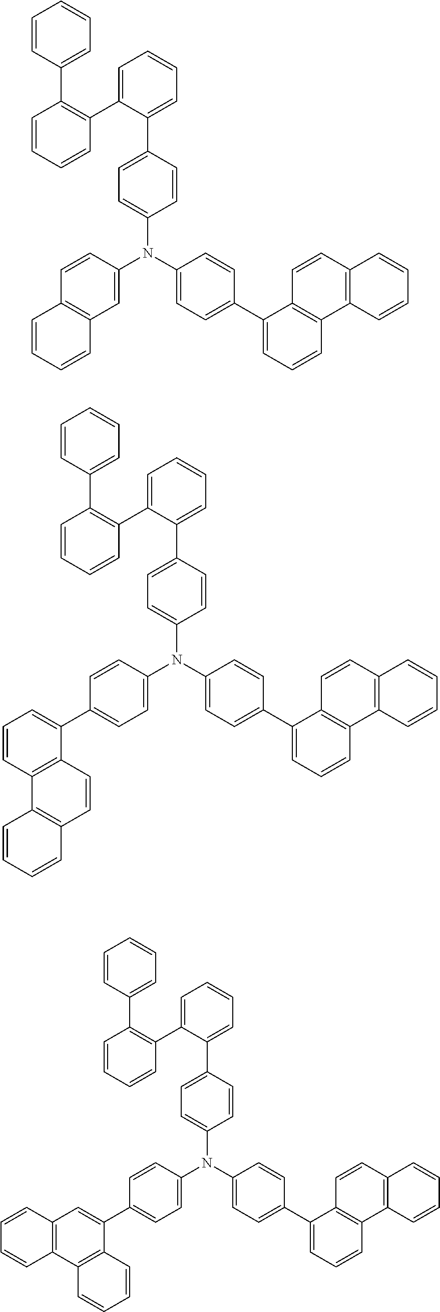 [SMILES: c1ccc(-c2ccccc2-c2ccccc2-c2ccc(N(c3ccc(-c4cccc5c4ccc4ccccc45)cc3)c3ccc4ccccc4c3)cc2)cc1 ; c1ccc(-c2ccccc2-c2ccccc2-c2ccc(N(c3ccc(-c4cccc5c4ccc4ccccc45)cc3)c3ccc(-c4cccc5c4ccc4ccccc45)cc3)cc2)cc1 ; c1ccc(-c2ccccc2-c2ccccc2-c2ccc(N(c3ccc(-c4cccc5c4ccc4ccccc45)cc3)c3ccc(-c4cc5ccccc5c5ccccc45)cc3)cc2)cc1]

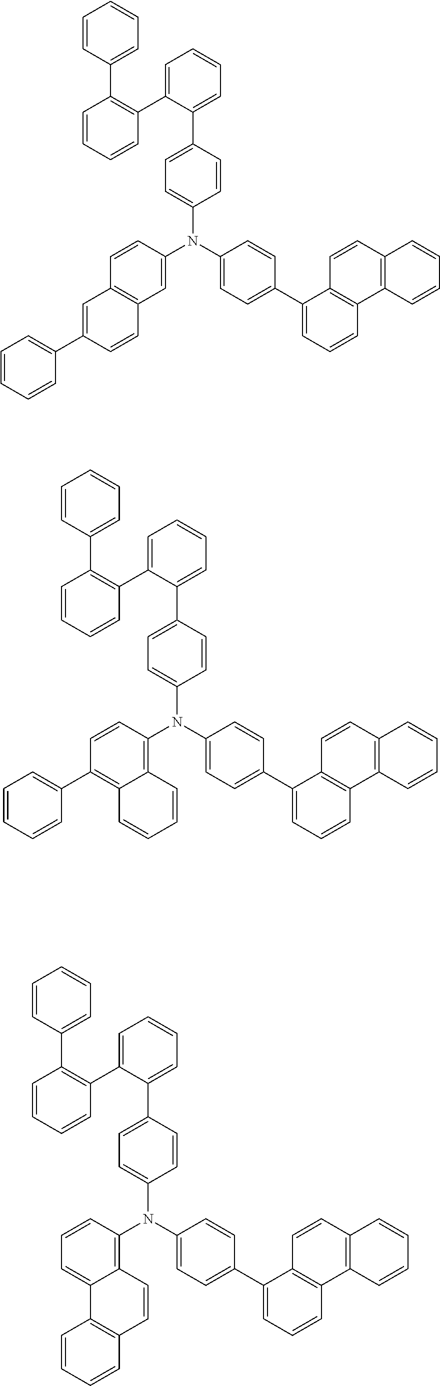 [SMILES: c1ccc(-c2ccc3cc(N(c4ccc(-c5ccccc5-c5ccccc5-c5ccccc5)cc4)c4ccc(-c5cccc6c5ccc5ccccc56)cc4)ccc3c2)cc1 ; c1ccc(-c2ccccc2-c2ccccc2-c2ccc(N(c3ccc(-c4cccc5c4ccc4ccccc45)cc3)c3ccc(-c4ccccc4)c4ccccc34)cc2)cc1 ; c1ccc(-c2ccccc2-c2ccccc2-c2ccc(N(c3ccc(-c4cccc5c4ccc4ccccc45)cc3)c3cccc4c3ccc3ccccc34)cc2)cc1]

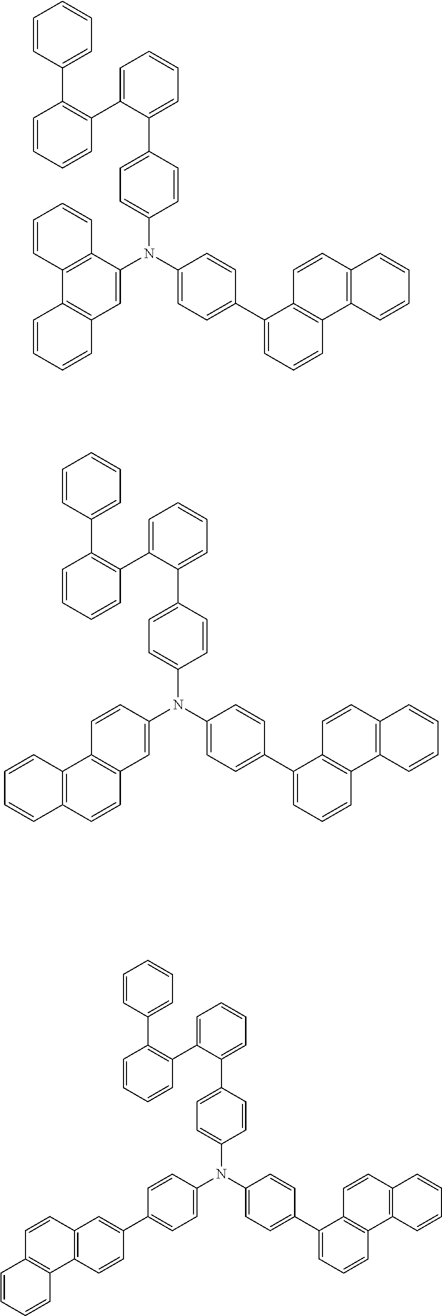 [SMILES: c1ccc(-c2ccccc2-c2ccccc2-c2ccc(N(c3ccc(-c4cccc5c4ccc4ccccc45)cc3)c3cc4ccccc4c4ccccc34)cc2)cc1 ; c1ccc(-c2ccccc2-c2ccccc2-c2ccc(N(c3ccc(-c4cccc5c4ccc4ccccc45)cc3)c3ccc4c(ccc5ccccc54)c3)cc2)cc1 ; c1ccc(-c2ccccc2-c2ccccc2-c2ccc(N(c3ccc(-c4ccc5c(ccc6ccccc65)c4)cc3)c3ccc(-c4cccc5c4ccc4ccccc45)cc3)cc2)cc1]

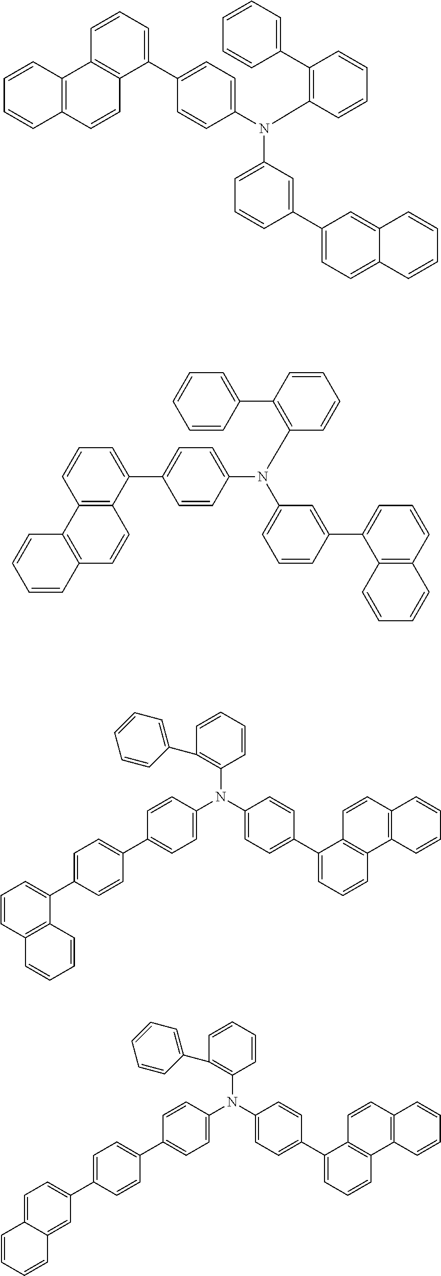 [SMILES: c1ccc(-c2ccccc2N(c2ccc(-c3cccc4c3ccc3ccccc34)cc2)c2cccc(-c3ccc4ccccc4c3)c2)cc1 ; c1ccc(-c2ccccc2N(c2ccc(-c3cccc4c3ccc3ccccc34)cc2)c2cccc(-c3cccc4ccccc34)c2)cc1 ; c1ccc(-c2ccccc2N(c2ccc(-c3ccc(-c4cccc5ccccc45)cc3)cc2)c2ccc(-c3cccc4c3ccc3ccccc34)cc2)cc1 ; c1ccc(-c2ccccc2N(c2ccc(-c3ccc(-c4ccc5ccccc5c4)cc3)cc2)c2ccc(-c3cccc4c3ccc3ccccc34)cc2)cc1]

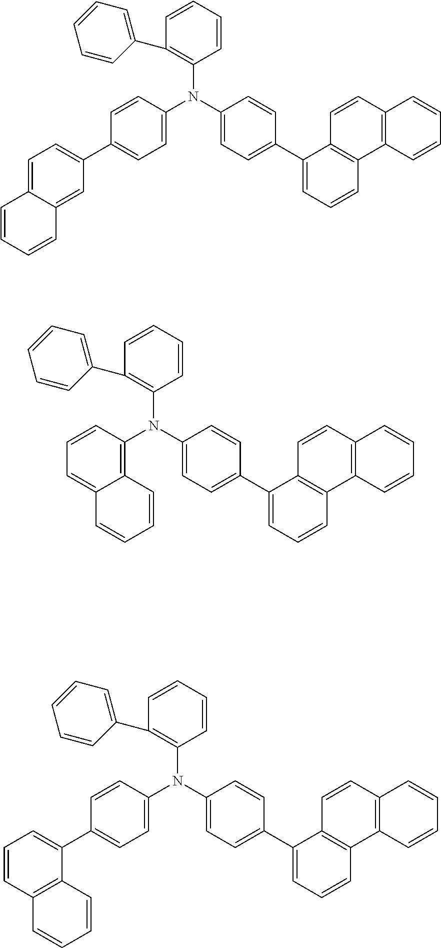 [SMILES: c1ccc(-c2ccccc2N(c2ccc(-c3ccc4ccccc4c3)cc2)c2ccc(-c3cccc4c3ccc3ccccc34)cc2)cc1 ; c1ccc(-c2ccccc2N(c2ccc(-c3cccc4c3ccc3ccccc34)cc2)c2cccc3ccccc23)cc1 ; c1ccc(-c2ccccc2N(c2ccc(-c3cccc4ccccc34)cc2)c2ccc(-c3cccc4c3ccc3ccccc34)cc2)cc1]

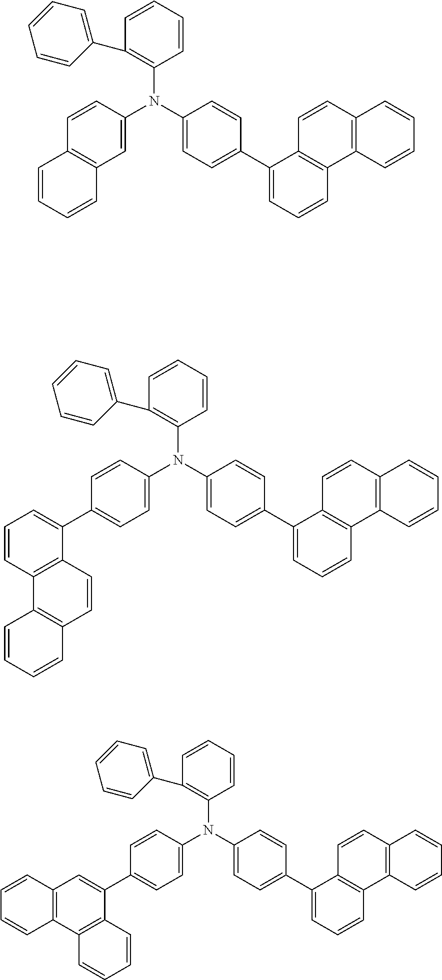 [SMILES: c1ccc(-c2ccccc2N(c2ccc(-c3cccc4c3ccc3ccccc34)cc2)c2ccc3ccccc3c2)cc1 ; c1ccc(-c2ccccc2N(c2ccc(-c3cccc4c3ccc3ccccc34)cc2)c2ccc(-c3cccc4c3ccc3ccccc34)cc2)cc1 ; c1ccc(-c2ccccc2N(c2ccc(-c3cccc4c3ccc3ccccc34)cc2)c2ccc(-c3cc4ccccc4c4ccccc34)cc2)cc1]

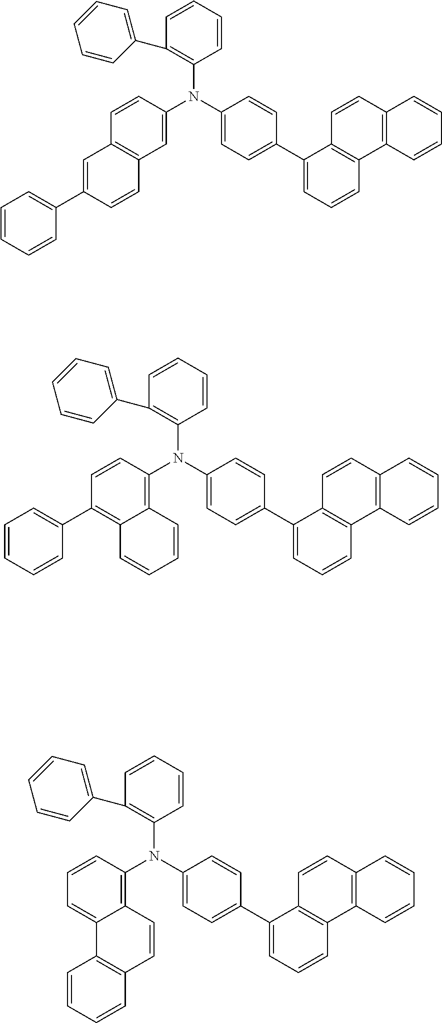 [SMILES: c1ccc(-c2ccc3cc(N(c4ccc(-c5cccc6c5ccc5ccccc56)cc4)c4ccccc4-c4ccccc4)ccc3c2)cc1 ; c1ccc(-c2ccccc2N(c2ccc(-c3cccc4c3ccc3ccccc34)cc2)c2ccc(-c3ccccc3)c3ccccc23)cc1 ; c1ccc(-c2ccccc2N(c2ccc(-c3cccc4c3ccc3ccccc34)cc2)c2cccc3c2ccc2ccccc23)cc1]

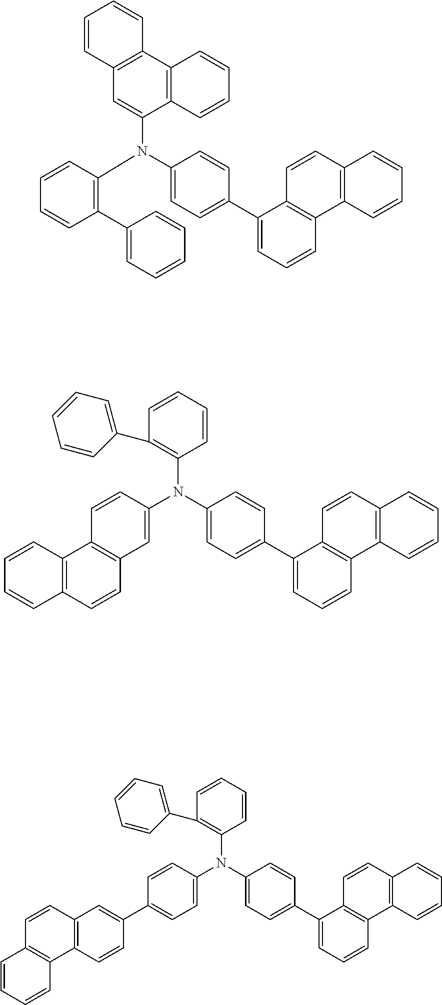 [SMILES: c1ccc(-c2ccccc2N(c2ccc(-c3cccc4c3ccc3ccccc34)cc2)c2cc3ccccc3c3ccccc23)cc1 ; c1ccc(-c2ccccc2N(c2ccc(-c3cccc4c3ccc3ccccc34)cc2)c2ccc3c(ccc4ccccc43)c2)cc1 ; c1ccc(-c2ccccc2N(c2ccc(-c3ccc4c(ccc5ccccc54)c3)cc2)c2ccc(-c3cccc4c3ccc3ccccc34)cc2)cc1]

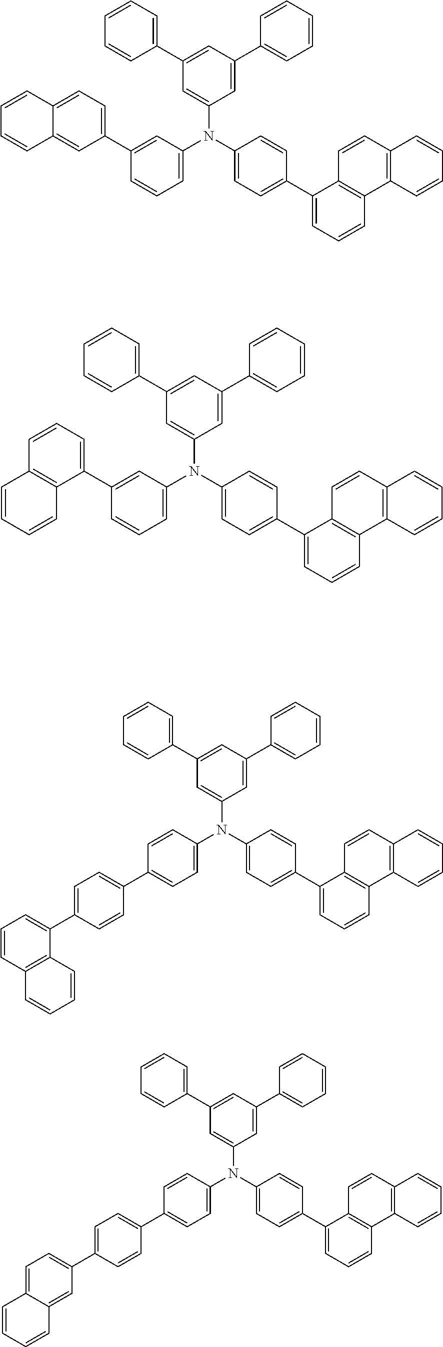 [SMILES: c1ccc(-c2cc(-c3ccccc3)cc(N(c3ccc(-c4cccc5c4ccc4ccccc45)cc3)c3cccc(-c4ccc5ccccc5c4)c3)c2)cc1 ; c1ccc(-c2cc(-c3ccccc3)cc(N(c3ccc(-c4cccc5c4ccc4ccccc45)cc3)c3cccc(-c4cccc5ccccc45)c3)c2)cc1 ; c1ccc(-c2cc(-c3ccccc3)cc(N(c3ccc(-c4ccc(-c5cccc6ccccc56)cc4)cc3)c3ccc(-c4cccc5c4ccc4ccccc45)cc3)c2)cc1 ; c1ccc(-c2cc(-c3ccccc3)cc(N(c3ccc(-c4ccc(-c5ccc6ccccc6c5)cc4)cc3)c3ccc(-c4cccc5c4ccc4ccccc45)cc3)c2)cc1]

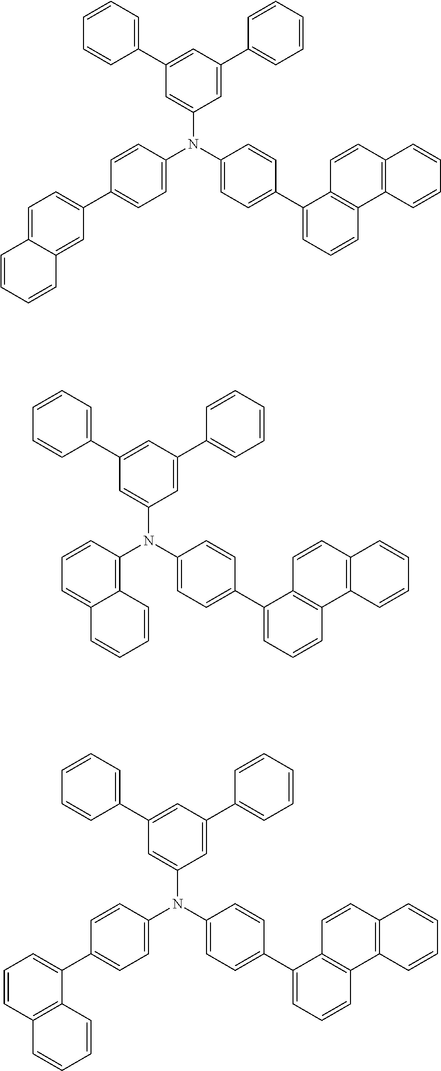 [SMILES: c1ccc(-c2cc(-c3ccccc3)cc(N(c3ccc(-c4ccc5ccccc5c4)cc3)c3ccc(-c4cccc5c4ccc4ccccc45)cc3)c2)cc1 ; c1ccc(-c2cc(-c3ccccc3)cc(N(c3ccc(-c4cccc5c4ccc4ccccc45)cc3)c3cccc4ccccc34)c2)cc1 ; c1ccc(-c2cc(-c3ccccc3)cc(N(c3ccc(-c4cccc5ccccc45)cc3)c3ccc(-c4cccc5c4ccc4ccccc45)cc3)c2)cc1]

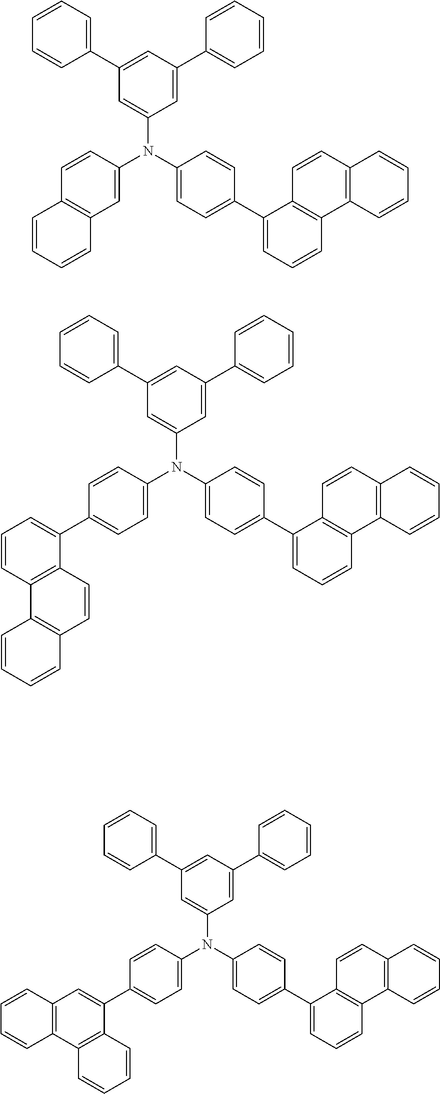 [SMILES: c1ccc(-c2cc(-c3ccccc3)cc(N(c3ccc(-c4cccc5c4ccc4ccccc45)cc3)c3ccc4ccccc4c3)c2)cc1 ; c1ccc(-c2cc(-c3ccccc3)cc(N(c3ccc(-c4cccc5c4ccc4ccccc45)cc3)c3ccc(-c4cccc5c4ccc4ccccc45)cc3)c2)cc1 ; c1ccc(-c2cc(-c3ccccc3)cc(N(c3ccc(-c4cccc5c4ccc4ccccc45)cc3)c3ccc(-c4cc5ccccc5c5ccccc45)cc3)c2)cc1]

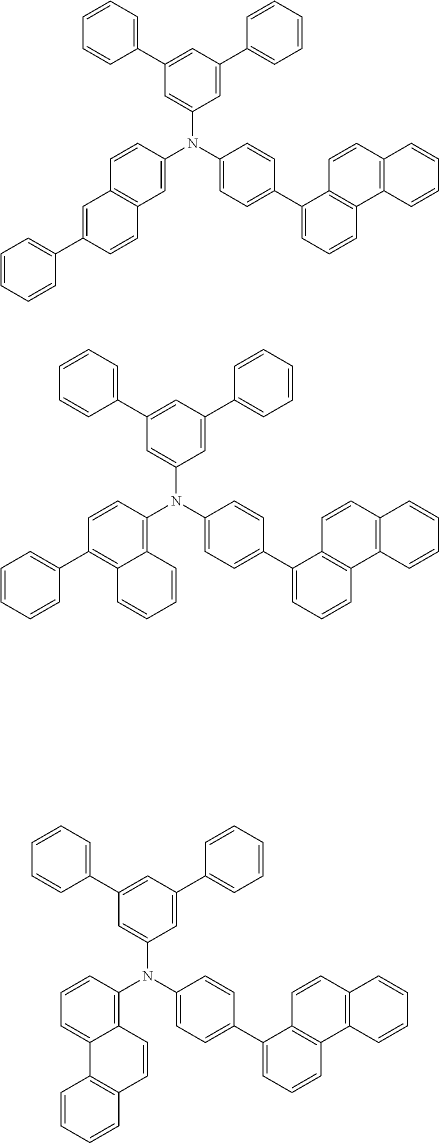 [SMILES: c1ccc(-c2cc(-c3ccccc3)cc(N(c3ccc(-c4cccc5c4ccc4ccccc45)cc3)c3ccc4cc(-c5ccccc5)ccc4c3)c2)cc1 ; c1ccc(-c2cc(-c3ccccc3)cc(N(c3ccc(-c4cccc5c4ccc4ccccc45)cc3)c3ccc(-c4ccccc4)c4ccccc34)c2)cc1 ; c1ccc(-c2cc(-c3ccccc3)cc(N(c3ccc(-c4cccc5c4ccc4ccccc45)cc3)c3cccc4c3ccc3ccccc34)c2)cc1]

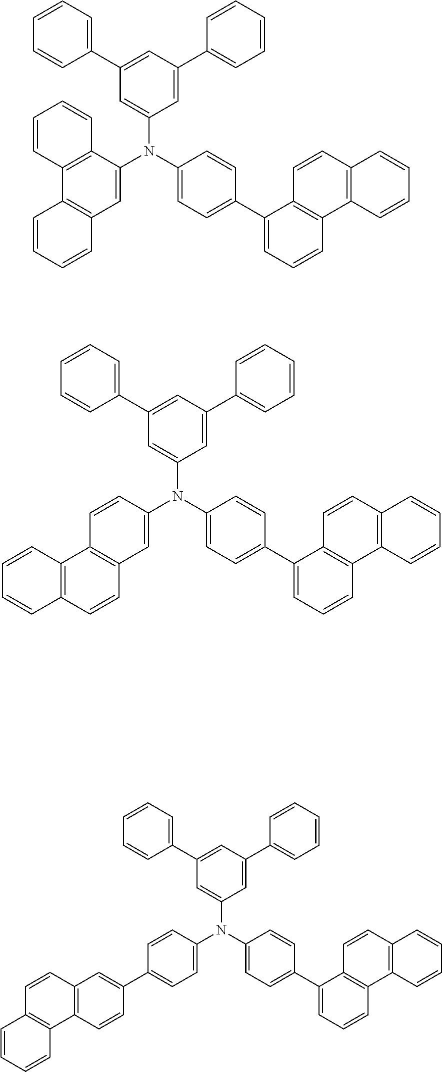 [SMILES: c1ccc(-c2cc(-c3ccccc3)cc(N(c3ccc(-c4cccc5c4ccc4ccccc45)cc3)c3cc4ccccc4c4ccccc34)c2)cc1 ; c1ccc(-c2cc(-c3ccccc3)cc(N(c3ccc(-c4cccc5c4ccc4ccccc45)cc3)c3ccc4c(ccc5ccccc54)c3)c2)cc1 ; c1ccc(-c2cc(-c3ccccc3)cc(N(c3ccc(-c4ccc5c(ccc6ccccc65)c4)cc3)c3ccc(-c4cccc5c4ccc4ccccc45)cc3)c2)cc1]

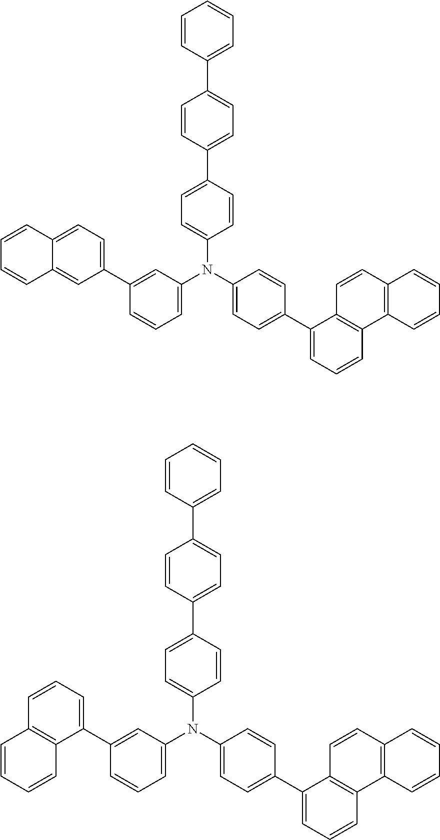 [SMILES: c1ccc(-c2ccc(-c3ccc(N(c4ccc(-c5cccc6c5ccc5ccccc56)cc4)c4cccc(-c5ccc6ccccc6c5)c4)cc3)cc2)cc1 ; c1ccc(-c2ccc(-c3ccc(N(c4ccc(-c5cccc6c5ccc5ccccc56)cc4)c4cccc(-c5cccc6ccccc56)c4)cc3)cc2)cc1]

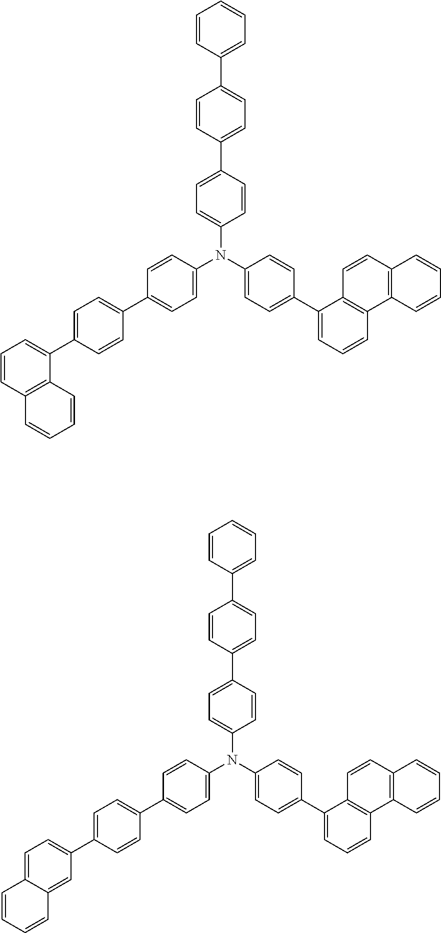 [SMILES: c1ccc(-c2ccc(-c3ccc(N(c4ccc(-c5ccc(-c6cccc7ccccc67)cc5)cc4)c4ccc(-c5cccc6c5ccc5ccccc56)cc4)cc3)cc2)cc1 ; c1ccc(-c2ccc(-c3ccc(N(c4ccc(-c5ccc(-c6ccc7ccccc7c6)cc5)cc4)c4ccc(-c5cccc6c5ccc5ccccc56)cc4)cc3)cc2)cc1]

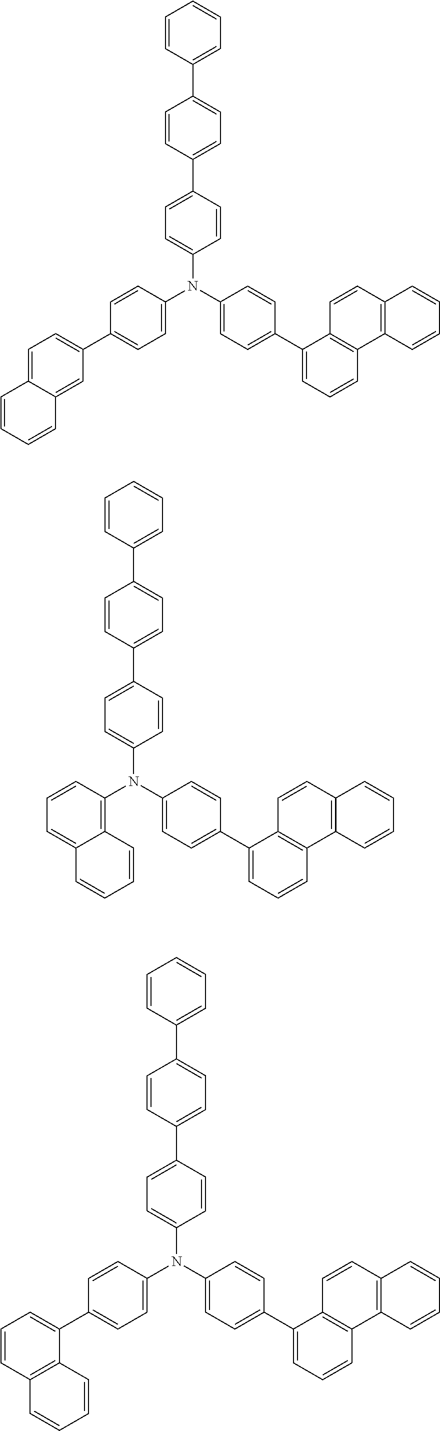 [SMILES: c1ccc(-c2ccc(-c3ccc(N(c4ccc(-c5ccc6ccccc6c5)cc4)c4ccc(-c5cccc6c5ccc5ccccc56)cc4)cc3)cc2)cc1 ; c1ccc(-c2ccc(-c3ccc(N(c4ccc(-c5cccc6c5ccc5ccccc56)cc4)c4cccc5ccccc45)cc3)cc2)cc1 ; c1ccc(-c2ccc(-c3ccc(N(c4ccc(-c5cccc6ccccc56)cc4)c4ccc(-c5cccc6c5ccc5ccccc56)cc4)cc3)cc2)cc1]

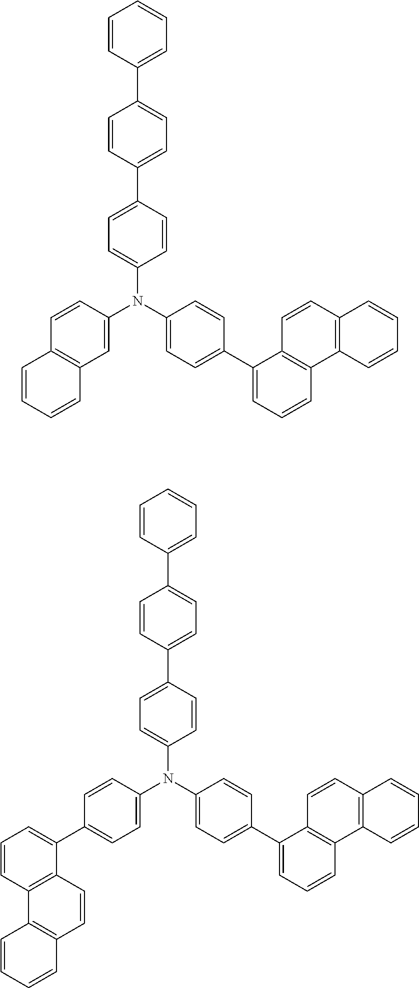 [SMILES: c1ccc(-c2ccc(-c3ccc(N(c4ccc(-c5cccc6c5ccc5ccccc56)cc4)c4ccc5ccccc5c4)cc3)cc2)cc1 ; c1ccc(-c2ccc(-c3ccc(N(c4ccc(-c5cccc6c5ccc5ccccc56)cc4)c4ccc(-c5cccc6c5ccc5ccccc56)cc4)cc3)cc2)cc1]

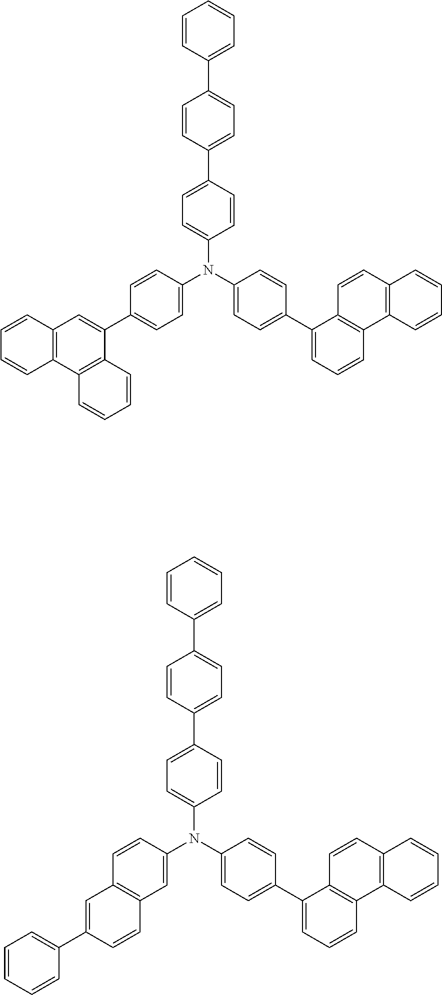 [SMILES: c1ccc(-c2ccc(-c3ccc(N(c4ccc(-c5cccc6c5ccc5ccccc56)cc4)c4ccc(-c5cc6ccccc6c6ccccc56)cc4)cc3)cc2)cc1 ; c1ccc(-c2ccc(-c3ccc(N(c4ccc(-c5cccc6c5ccc5ccccc56)cc4)c4ccc5cc(-c6ccccc6)ccc5c4)cc3)cc2)cc1]

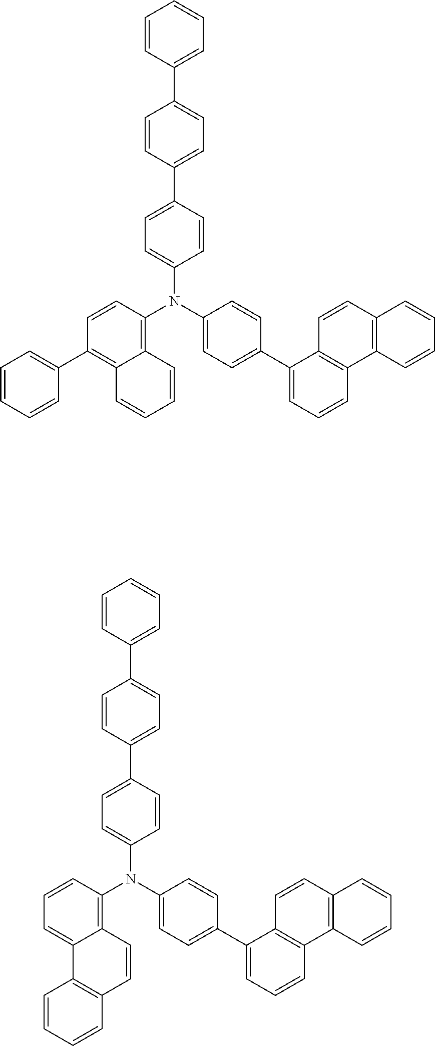 [SMILES: c1ccc(-c2ccc(-c3ccc(N(c4ccc(-c5cccc6c5ccc5ccccc56)cc4)c4ccc(-c5ccccc5)c5ccccc45)cc3)cc2)cc1 ; c1ccc(-c2ccc(-c3ccc(N(c4ccc(-c5cccc6c5ccc5ccccc56)cc4)c4cccc5c4ccc4ccccc45)cc3)cc2)cc1]

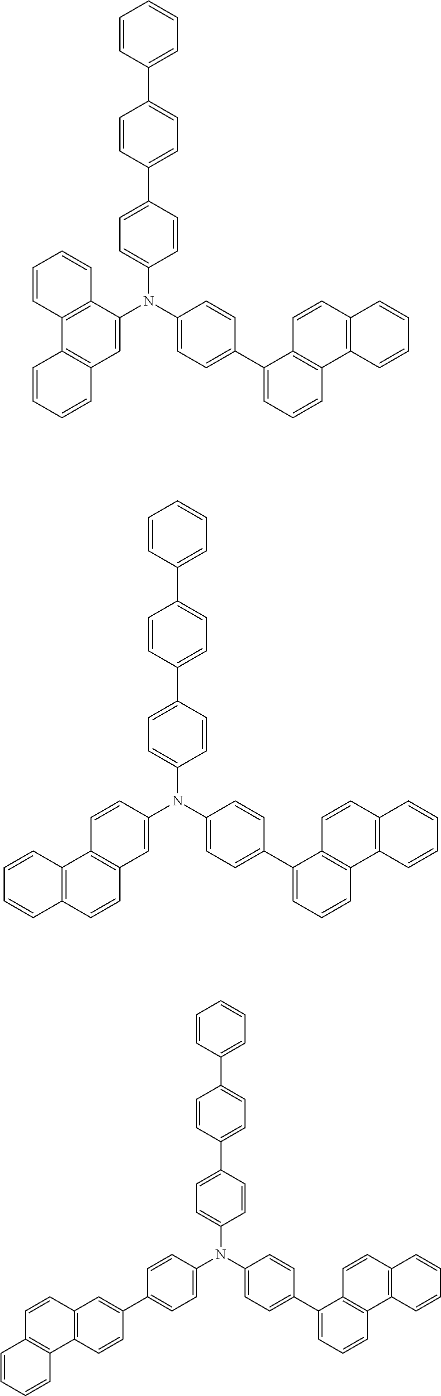 [SMILES: c1ccc(-c2ccc(-c3ccc(N(c4ccc(-c5cccc6c5ccc5ccccc56)cc4)c4cc5ccccc5c5ccccc45)cc3)cc2)cc1 ; c1ccc(-c2ccc(-c3ccc(N(c4ccc(-c5cccc6c5ccc5ccccc56)cc4)c4ccc5c(ccc6ccccc65)c4)cc3)cc2)cc1 ; c1ccc(-c2ccc(-c3ccc(N(c4ccc(-c5ccc6c(ccc7ccccc76)c5)cc4)c4ccc(-c5cccc6c5ccc5ccccc56)cc4)cc3)cc2)cc1]

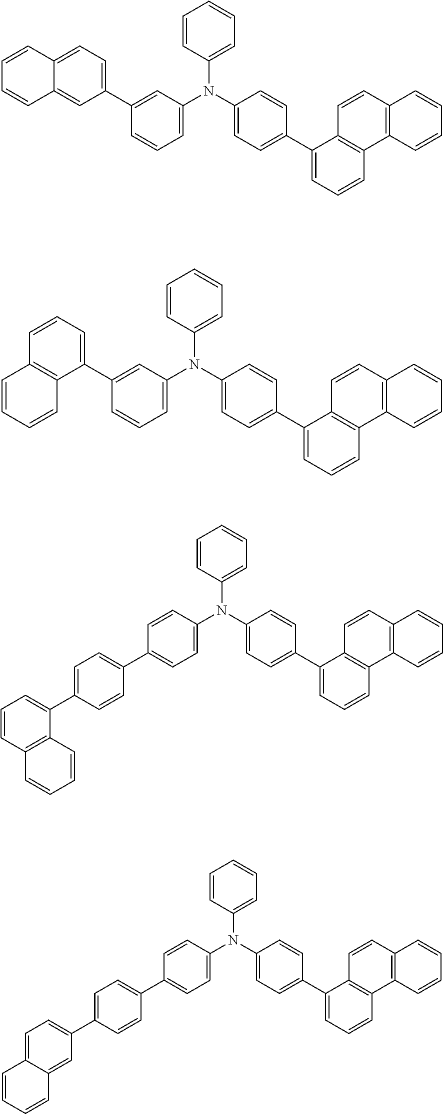 [SMILES: c1ccc(N(c2ccc(-c3cccc4c3ccc3ccccc34)cc2)c2cccc(-c3ccc4ccccc4c3)c2)cc1 ; c1ccc(N(c2ccc(-c3cccc4c3ccc3ccccc34)cc2)c2cccc(-c3cccc4ccccc34)c2)cc1 ; c1ccc(N(c2ccc(-c3ccc(-c4cccc5ccccc45)cc3)cc2)c2ccc(-c3cccc4c3ccc3ccccc34)cc2)cc1 ; c1ccc(N(c2ccc(-c3ccc(-c4ccc5ccccc5c4)cc3)cc2)c2ccc(-c3cccc4c3ccc3ccccc34)cc2)cc1]

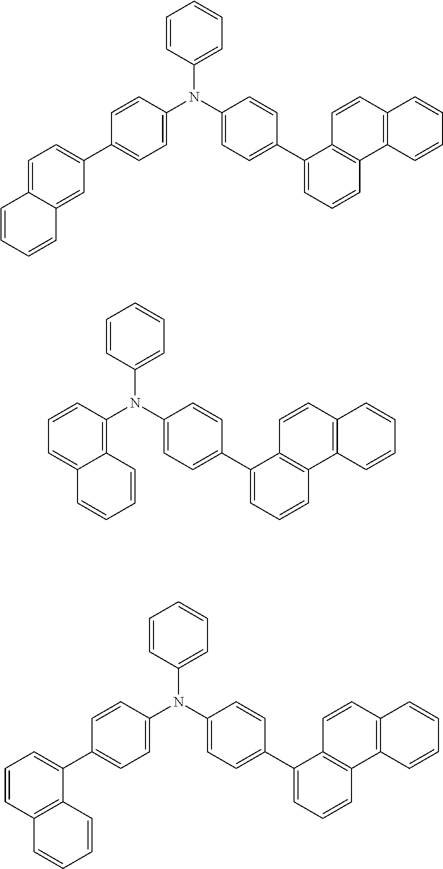 [SMILES: c1ccc(N(c2ccc(-c3ccc4ccccc4c3)cc2)c2ccc(-c3cccc4c3ccc3ccccc34)cc2)cc1 ; c1ccc(N(c2ccc(-c3cccc4c3ccc3ccccc34)cc2)c2cccc3ccccc23)cc1 ; c1ccc(N(c2ccc(-c3cccc4ccccc34)cc2)c2ccc(-c3cccc4c3ccc3ccccc34)cc2)cc1]

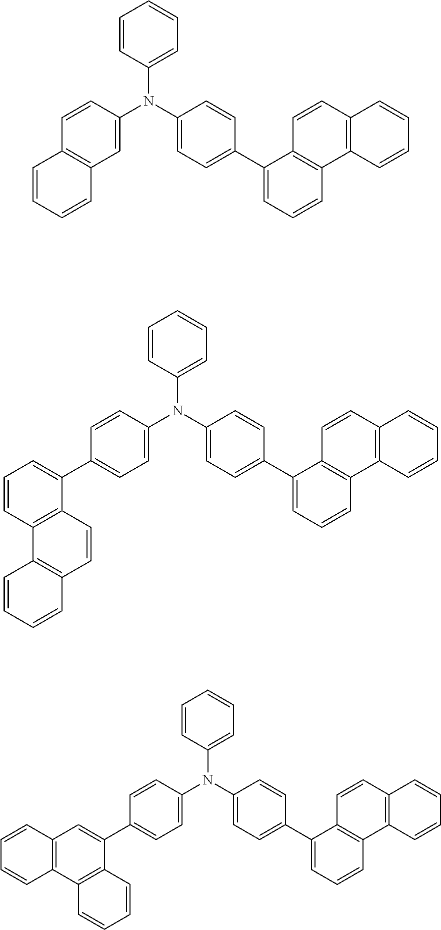 [SMILES: c1ccc(N(c2ccc(-c3cccc4c3ccc3ccccc34)cc2)c2ccc3ccccc3c2)cc1 ; c1ccc(N(c2ccc(-c3cccc4c3ccc3ccccc34)cc2)c2ccc(-c3cccc4c3ccc3ccccc34)cc2)cc1 ; c1ccc(N(c2ccc(-c3cccc4c3ccc3ccccc34)cc2)c2ccc(-c3cc4ccccc4c4ccccc34)cc2)cc1]

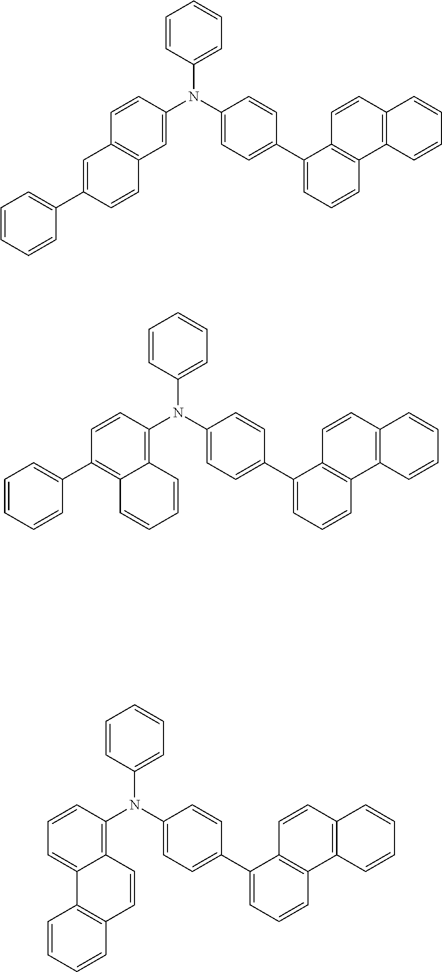 [SMILES: c1ccc(-c2ccc3cc(N(c4ccccc4)c4ccc(-c5cccc6c5ccc5ccccc56)cc4)ccc3c2)cc1 ; c1ccc(-c2ccc(N(c3ccccc3)c3ccc(-c4cccc5c4ccc4ccccc45)cc3)c3ccccc23)cc1 ; c1ccc(N(c2ccc(-c3cccc4c3ccc3ccccc34)cc2)c2cccc3c2ccc2ccccc23)cc1]

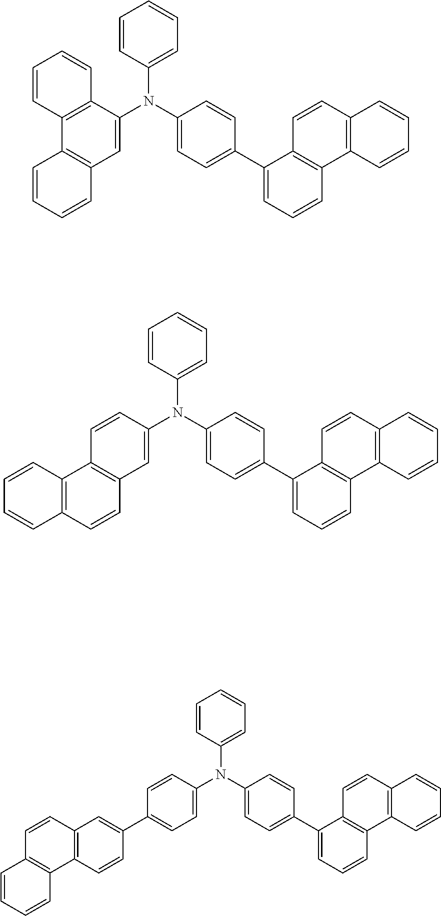 [SMILES: c1ccc(N(c2ccc(-c3cccc4c3ccc3ccccc34)cc2)c2cc3ccccc3c3ccccc23)cc1 ; c1ccc(N(c2ccc(-c3cccc4c3ccc3ccccc34)cc2)c2ccc3c(ccc4ccccc43)c2)cc1 ; c1ccc(N(c2ccc(-c3ccc4c(ccc5ccccc54)c3)cc2)c2ccc(-c3cccc4c3ccc3ccccc34)cc2)cc1]

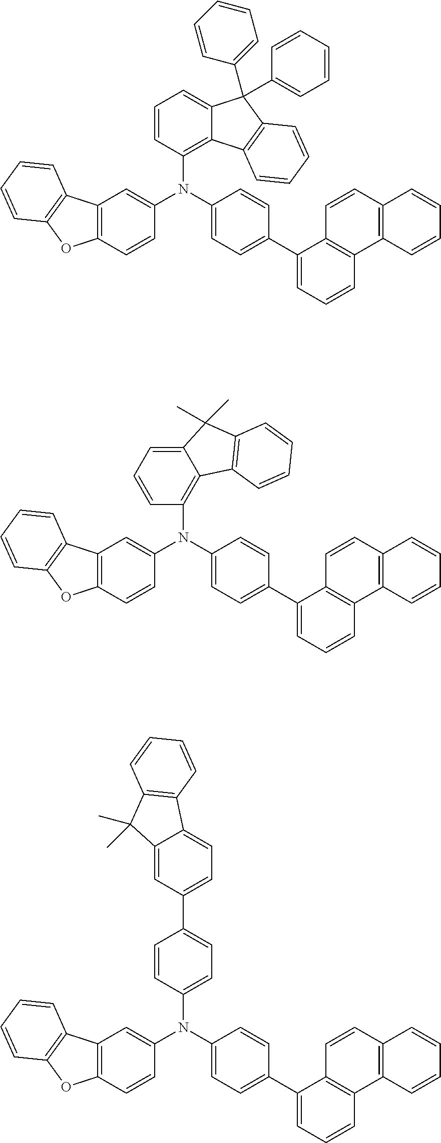 [SMILES: c1ccc(C2(c3ccccc3)c3ccccc3-c3c(N(c4ccc(-c5cccc6c5ccc5ccccc56)cc4)c4ccc5oc6ccccc6c5c4)cccc32)cc1 ; CC1(C)c2ccccc2-c2c(N(c3ccc(-c4cccc5c4ccc4ccccc45)cc3)c3ccc4oc5ccccc5c4c3)cccc21 ; CC1(C)c2ccccc2-c2ccc(-c3ccc(N(c4ccc(-c5cccc6c5ccc5ccccc56)cc4)c4ccc5oc6ccccc6c5c4)cc3)cc21]

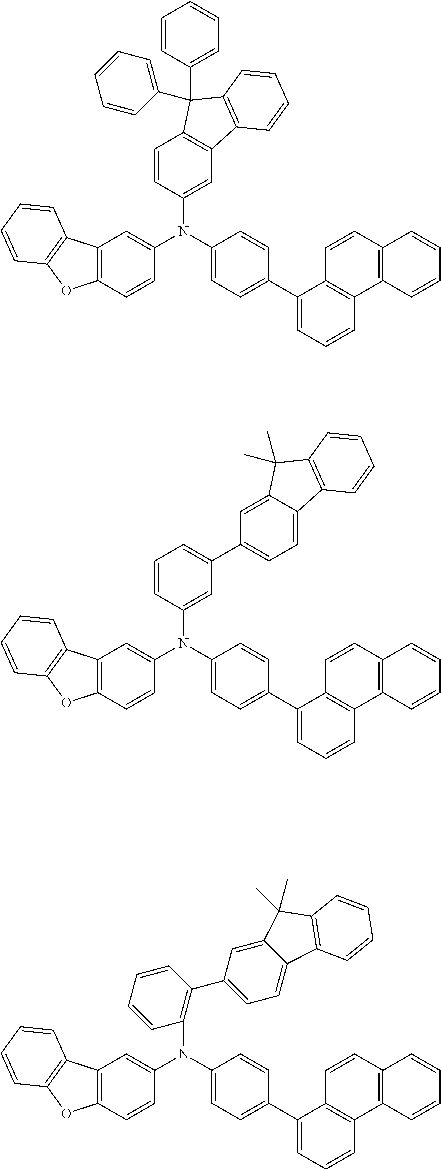 [SMILES: c1ccc(C2(c3ccccc3)c3ccccc3-c3cc(N(c4ccc(-c5cccc6c5ccc5ccccc56)cc4)c4ccc5oc6ccccc6c5c4)ccc32)cc1 ; CC1(C)c2ccccc2-c2ccc(-c3cccc(N(c4ccc(-c5cccc6c5ccc5ccccc56)cc4)c4ccc5oc6ccccc6c5c4)c3)cc21 ; CC1(C)c2ccccc2-c2ccc(-c3ccccc3N(c3ccc(-c4cccc5c4ccc4ccccc45)cc3)c3ccc4oc5ccccc5c4c3)cc21]

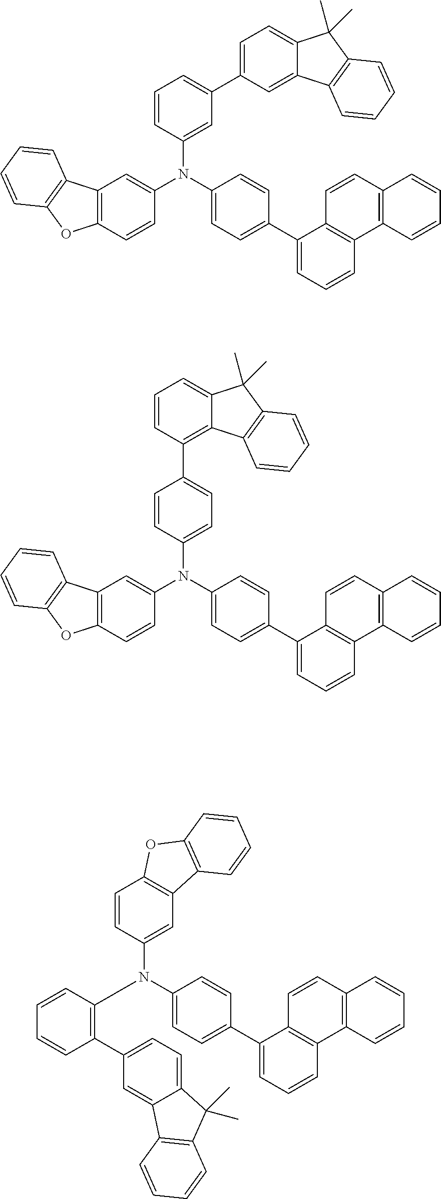 [SMILES: CC1(C)c2ccccc2-c2cc(-c3cccc(N(c4ccc(-c5cccc6c5ccc5ccccc56)cc4)c4ccc5oc6ccccc6c5c4)c3)ccc21 ; CC1(C)c2ccccc2-c2c(-c3ccc(N(c4ccc(-c5cccc6c5ccc5ccccc56)cc4)c4ccc5oc6ccccc6c5c4)cc3)cccc21 ; CC1(C)c2ccccc2-c2cc(-c3ccccc3N(c3ccc(-c4cccc5c4ccc4ccccc45)cc3)c3ccc4oc5ccccc5c4c3)ccc21]

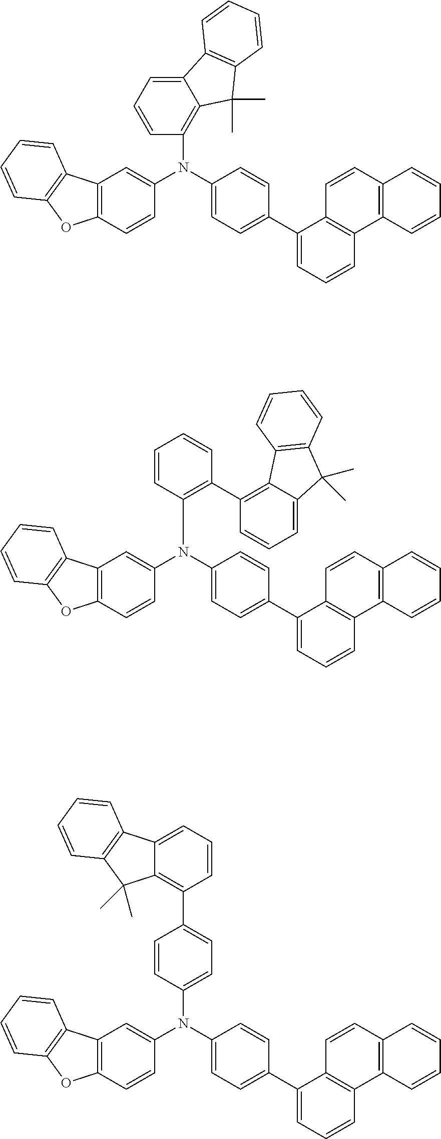 [SMILES: CC1(C)c2ccccc2-c2cccc(N(c3ccc(-c4cccc5c4ccc4ccccc45)cc3)c3ccc4oc5ccccc5c4c3)c21 ; CC1(C)c2ccccc2-c2c(-c3ccccc3N(c3ccc(-c4cccc5c4ccc4ccccc45)cc3)c3ccc4oc5ccccc5c4c3)cccc21 ; CC1(C)c2ccccc2-c2cccc(-c3ccc(N(c4ccc(-c5cccc6c5ccc5ccccc56)cc4)c4ccc5oc6ccccc6c5c4)cc3)c21]

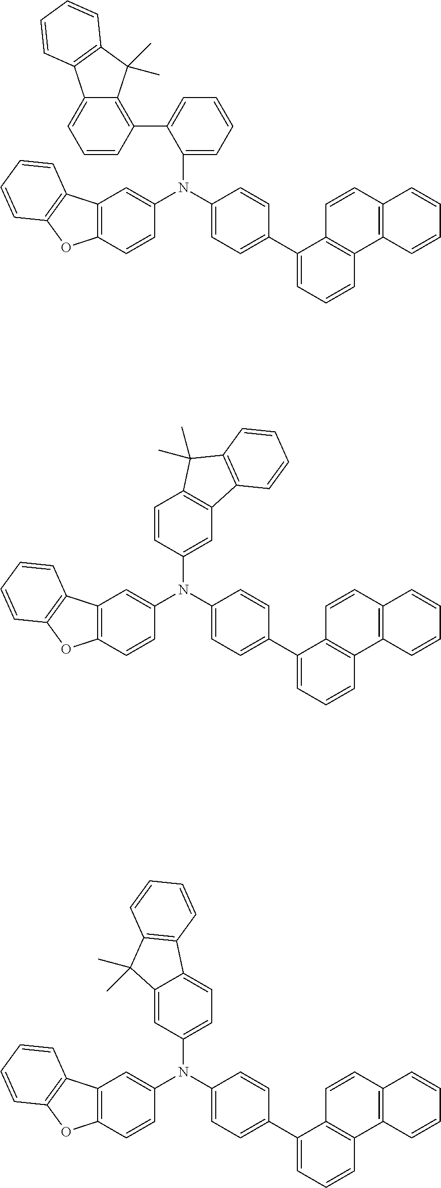 [SMILES: CC1(C)c2ccccc2-c2cccc(-c3ccccc3N(c3ccc(-c4cccc5c4ccc4ccccc45)cc3)c3ccc4oc5ccccc5c4c3)c21 ; CC1(C)c2ccccc2-c2cc(N(c3ccc(-c4cccc5c4ccc4ccccc45)cc3)c3ccc4oc5ccccc5c4c3)ccc21 ; CC1(C)c2ccccc2-c2ccc(N(c3ccc(-c4cccc5c4ccc4ccccc45)cc3)c3ccc4oc5ccccc5c4c3)cc21]

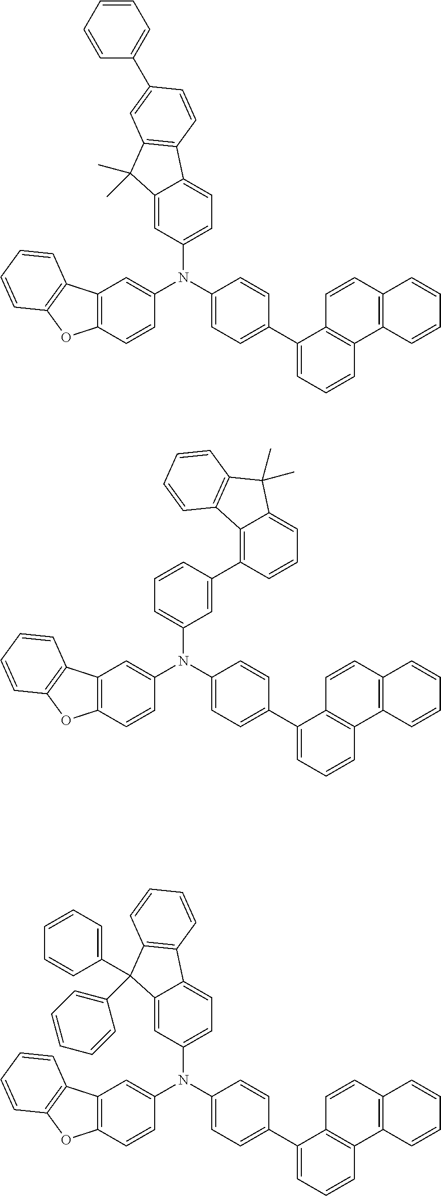 [SMILES: CC1(C)c2cc(-c3ccccc3)ccc2-c2ccc(N(c3ccc(-c4cccc5c4ccc4ccccc45)cc3)c3ccc4oc5ccccc5c4c3)cc21 ; CC1(C)c2ccccc2-c2c(-c3cccc(N(c4ccc(-c5cccc6c5ccc5ccccc56)cc4)c4ccc5oc6ccccc6c5c4)c3)cccc21 ; c1ccc(C2(c3ccccc3)c3ccccc3-c3ccc(N(c4ccc(-c5cccc6c5ccc5ccccc56)cc4)c4ccc5oc6ccccc6c5c4)cc32)cc1]

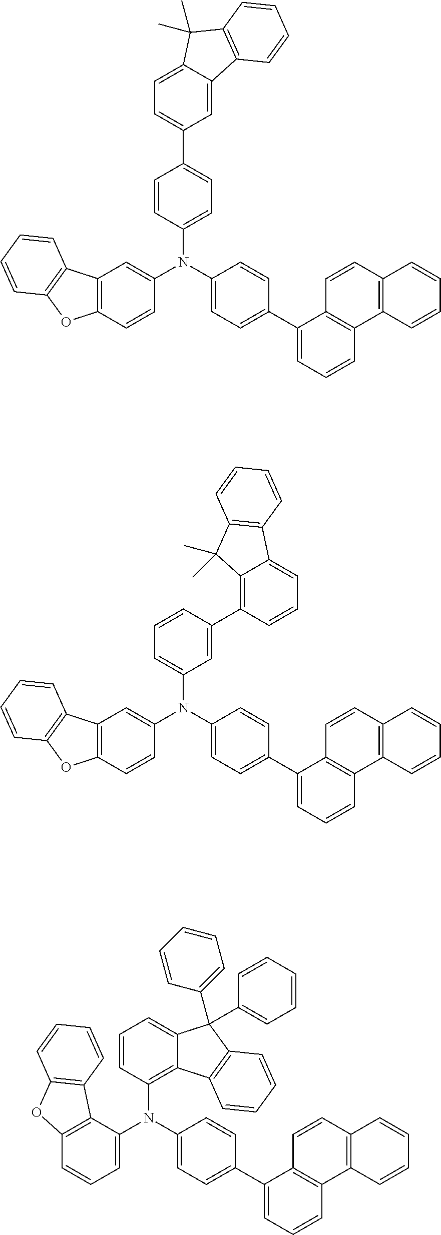 [SMILES: CC1(C)c2ccccc2-c2cc(-c3ccc(N(c4ccc(-c5cccc6c5ccc5ccccc56)cc4)c4ccc5oc6ccccc6c5c4)cc3)ccc21 ; CC1(C)c2ccccc2-c2cccc(-c3cccc(N(c4ccc(-c5cccc6c5ccc5ccccc56)cc4)c4ccc5oc6ccccc6c5c4)c3)c21 ; c1ccc(C2(c3ccccc3)c3ccccc3-c3c(N(c4ccc(-c5cccc6c5ccc5ccccc56)cc4)c4cccc5oc6ccccc6c45)cccc32)cc1]

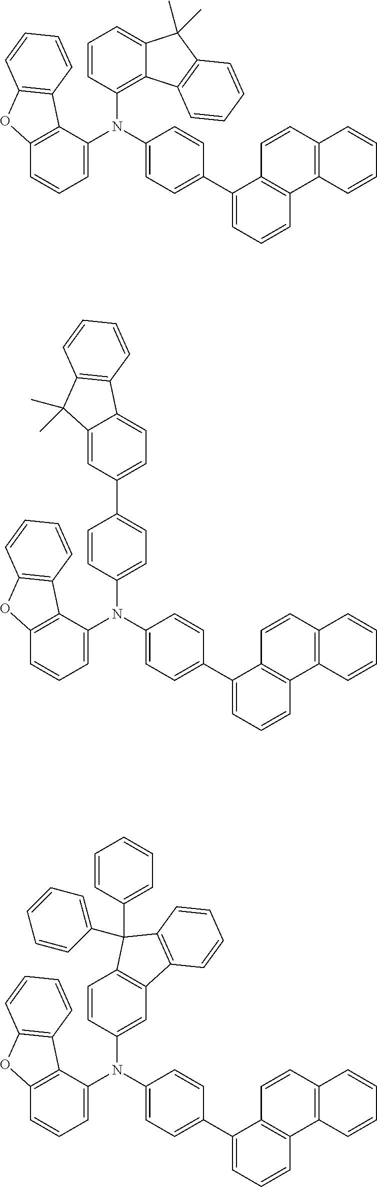 [SMILES: CC1(C)c2ccccc2-c2c(N(c3ccc(-c4cccc5c4ccc4ccccc45)cc3)c3cccc4oc5ccccc5c34)cccc21 ; CC1(C)c2ccccc2-c2ccc(-c3ccc(N(c4ccc(-c5cccc6c5ccc5ccccc56)cc4)c4cccc5oc6ccccc6c45)cc3)cc21 ; c1ccc(C2(c3ccccc3)c3ccccc3-c3cc(N(c4ccc(-c5cccc6c5ccc5ccccc56)cc4)c4cccc5oc6ccccc6c45)ccc32)cc1]

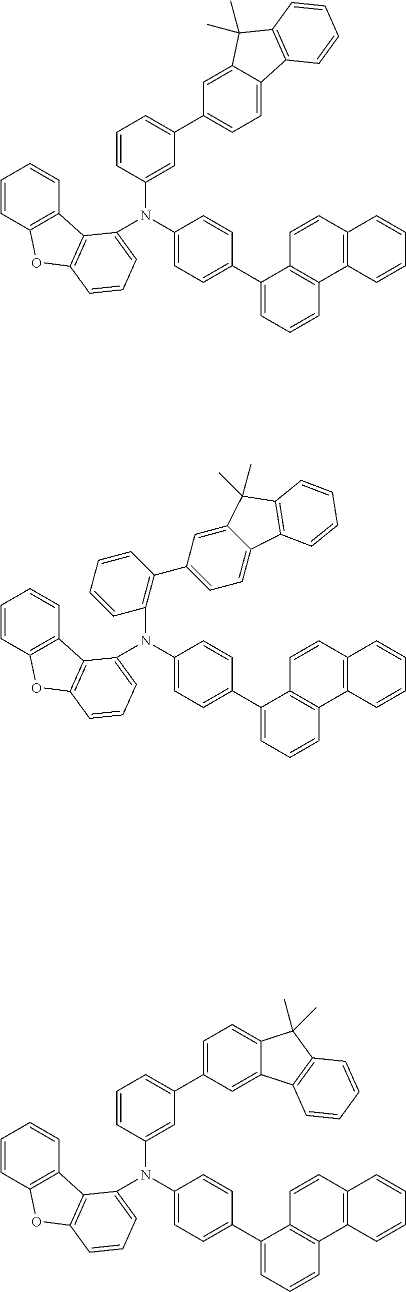 [SMILES: CC1(C)c2ccccc2-c2ccc(-c3cccc(N(c4ccc(-c5cccc6c5ccc5ccccc56)cc4)c4cccc5oc6ccccc6c45)c3)cc21 ; CC1(C)c2ccccc2-c2ccc(-c3ccccc3N(c3ccc(-c4cccc5c4ccc4ccccc45)cc3)c3cccc4oc5ccccc5c34)cc21 ; CC1(C)c2ccccc2-c2cc(-c3cccc(N(c4ccc(-c5cccc6c5ccc5ccccc56)cc4)c4cccc5oc6ccccc6c45)c3)ccc21]

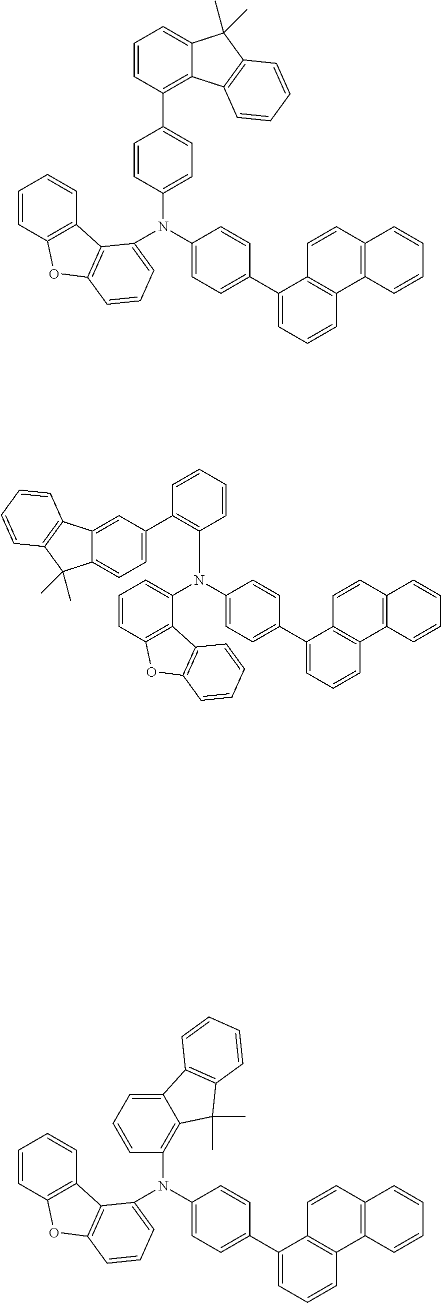 [SMILES: CC1(C)c2ccccc2-c2c(-c3ccc(N(c4ccc(-c5cccc6c5ccc5ccccc56)cc4)c4cccc5oc6ccccc6c45)cc3)cccc21 ; CC1(C)c2ccccc2-c2cc(-c3ccccc3N(c3ccc(-c4cccc5c4ccc4ccccc45)cc3)c3cccc4oc5ccccc5c34)ccc21 ; CC1(C)c2ccccc2-c2cccc(N(c3ccc(-c4cccc5c4ccc4ccccc45)cc3)c3cccc4oc5ccccc5c34)c21]

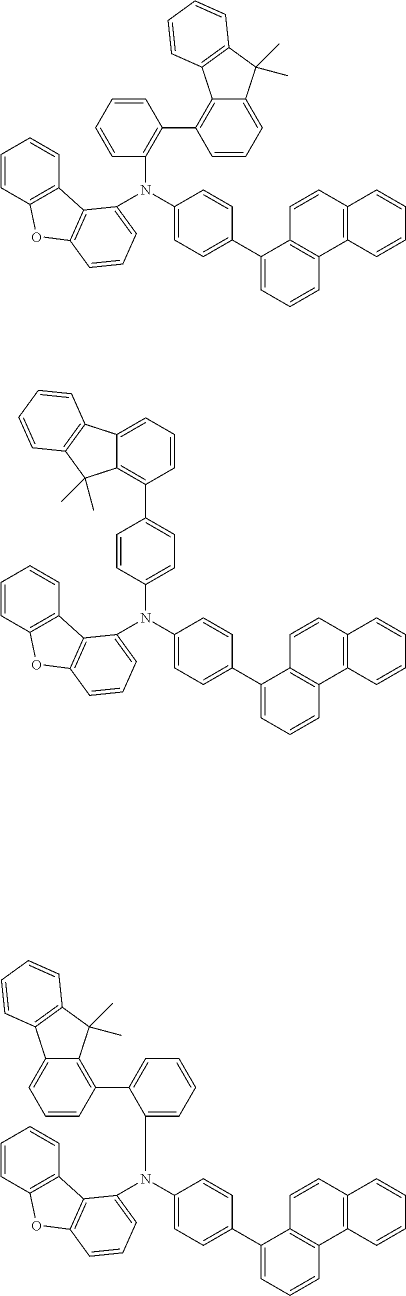 [SMILES: CC1(C)c2ccccc2-c2c(-c3ccccc3N(c3ccc(-c4cccc5c4ccc4ccccc45)cc3)c3cccc4oc5ccccc5c34)cccc21 ; CC1(C)c2ccccc2-c2cccc(-c3ccc(N(c4ccc(-c5cccc6c5ccc5ccccc56)cc4)c4cccc5oc6ccccc6c45)cc3)c21 ; CC1(C)c2ccccc2-c2cccc(-c3ccccc3N(c3ccc(-c4cccc5c4ccc4ccccc45)cc3)c3cccc4oc5ccccc5c34)c21]

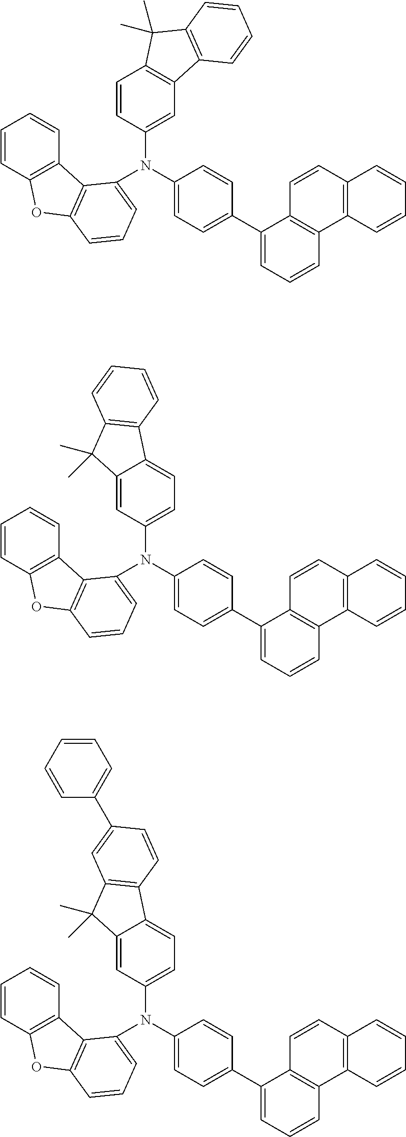 [SMILES: CC1(C)c2ccccc2-c2cc(N(c3ccc(-c4cccc5c4ccc4ccccc45)cc3)c3cccc4oc5ccccc5c34)ccc21 ; CC1(C)c2ccccc2-c2ccc(N(c3ccc(-c4cccc5c4ccc4ccccc45)cc3)c3cccc4oc5ccccc5c34)cc21 ; CC1(C)c2cc(-c3ccccc3)ccc2-c2ccc(N(c3ccc(-c4cccc5c4ccc4ccccc45)cc3)c3cccc4oc5ccccc5c34)cc21]

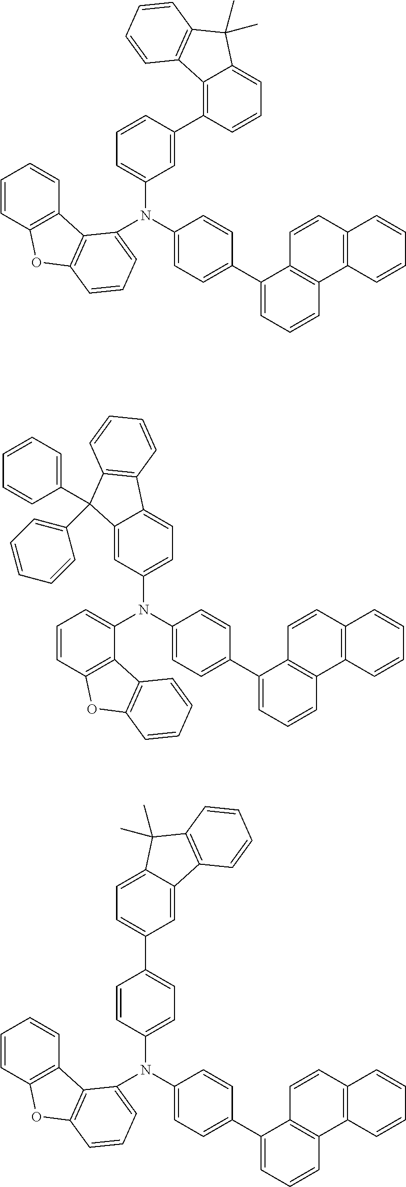 [SMILES: CC1(C)c2ccccc2-c2c(-c3cccc(N(c4ccc(-c5cccc6c5ccc5ccccc56)cc4)c4cccc5oc6ccccc6c45)c3)cccc21 ; c1ccc(C2(c3ccccc3)c3ccccc3-c3ccc(N(c4ccc(-c5cccc6c5ccc5ccccc56)cc4)c4cccc5oc6ccccc6c45)cc32)cc1 ; CC1(C)c2ccccc2-c2cc(-c3ccc(N(c4ccc(-c5cccc6c5ccc5ccccc56)cc4)c4cccc5oc6ccccc6c45)cc3)ccc21]

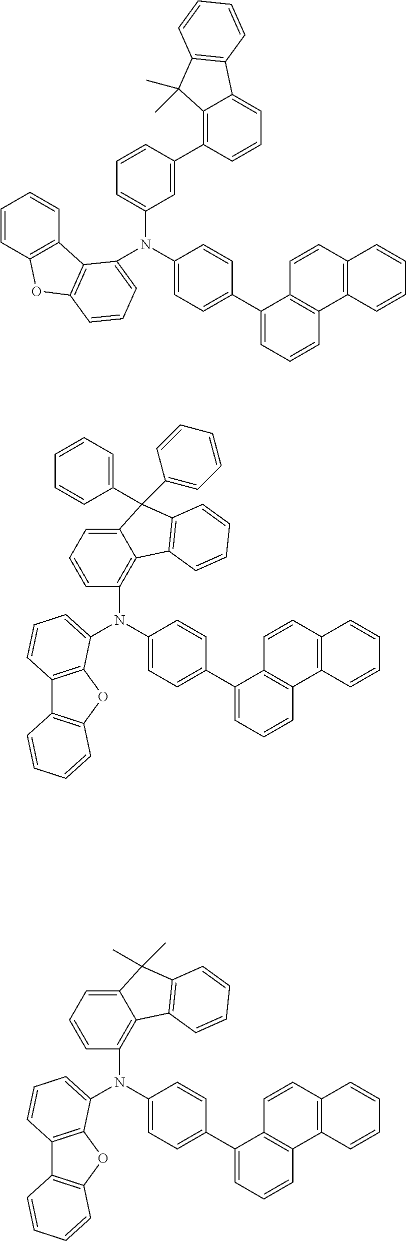 [SMILES: CC1(C)c2ccccc2-c2cccc(-c3cccc(N(c4ccc(-c5cccc6c5ccc5ccccc56)cc4)c4cccc5oc6ccccc6c45)c3)c21 ; c1ccc(C2(c3ccccc3)c3ccccc3-c3c(N(c4ccc(-c5cccc6c5ccc5ccccc56)cc4)c4cccc5c4oc4ccccc45)cccc32)cc1 ; CC1(C)c2ccccc2-c2c(N(c3ccc(-c4cccc5c4ccc4ccccc45)cc3)c3cccc4c3oc3ccccc34)cccc21]

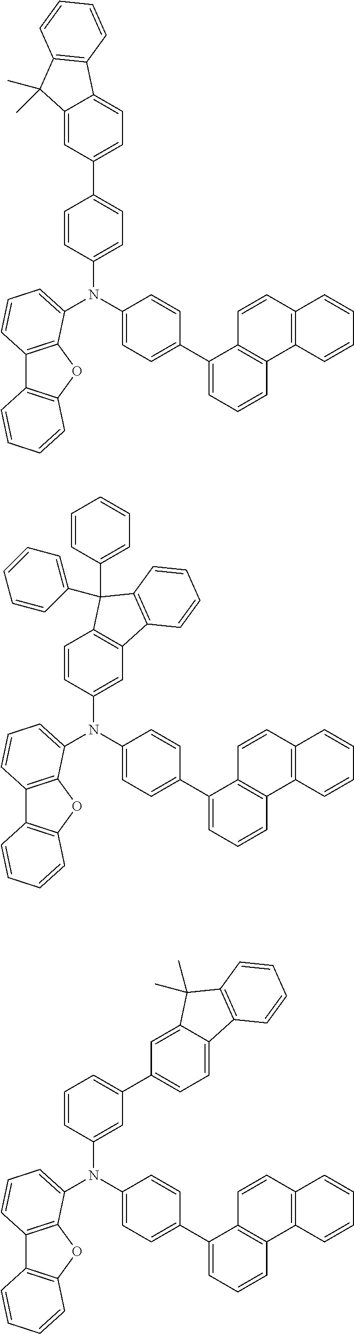 [SMILES: CC1(C)c2ccccc2-c2ccc(-c3ccc(N(c4ccc(-c5cccc6c5ccc5ccccc56)cc4)c4cccc5c4oc4ccccc45)cc3)cc21 ; c1ccc(C2(c3ccccc3)c3ccccc3-c3cc(N(c4ccc(-c5cccc6c5ccc5ccccc56)cc4)c4cccc5c4oc4ccccc45)ccc32)cc1 ; CC1(C)c2ccccc2-c2ccc(-c3cccc(N(c4ccc(-c5cccc6c5ccc5ccccc56)cc4)c4cccc5c4oc4ccccc45)c3)cc21]

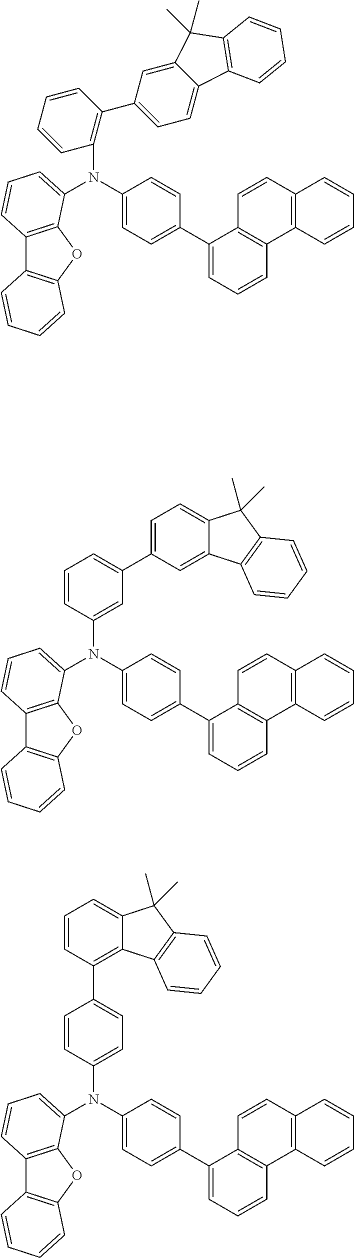 [SMILES: CC1(C)c2ccccc2-c2ccc(-c3ccccc3N(c3ccc(-c4cccc5c4ccc4ccccc45)cc3)c3cccc4c3oc3ccccc34)cc21 ; CC1(C)c2ccccc2-c2cc(-c3cccc(N(c4ccc(-c5cccc6c5ccc5ccccc56)cc4)c4cccc5c4oc4ccccc45)c3)ccc21 ; CC1(C)c2ccccc2-c2c(-c3ccc(N(c4ccc(-c5cccc6c5ccc5ccccc56)cc4)c4cccc5c4oc4ccccc45)cc3)cccc21]

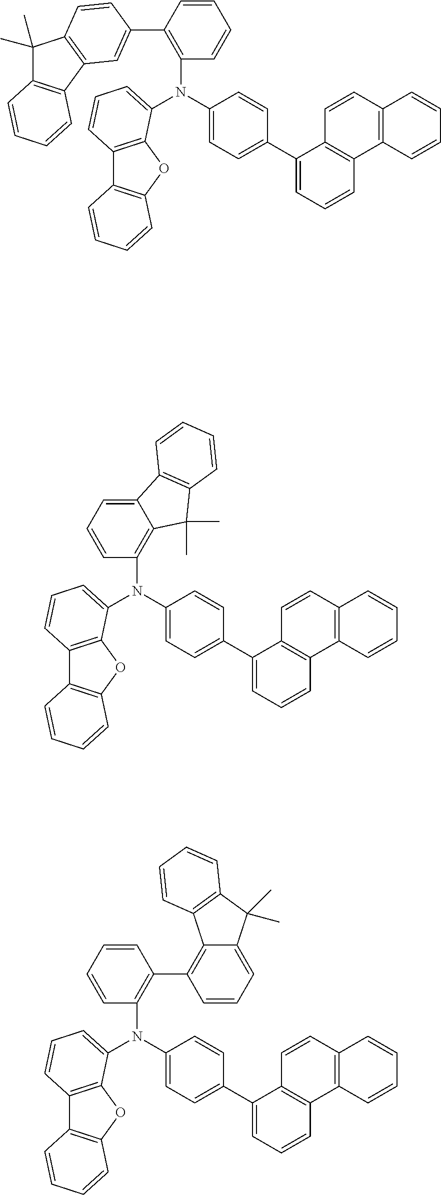 [SMILES: CC1(C)c2ccccc2-c2cc(-c3ccccc3N(c3ccc(-c4cccc5c4ccc4ccccc45)cc3)c3cccc4c3oc3ccccc34)ccc21 ; CC1(C)c2ccccc2-c2cccc(N(c3ccc(-c4cccc5c4ccc4ccccc45)cc3)c3cccc4c3oc3ccccc34)c21 ; CC1(C)c2ccccc2-c2c(-c3ccccc3N(c3ccc(-c4cccc5c4ccc4ccccc45)cc3)c3cccc4c3oc3ccccc34)cccc21]

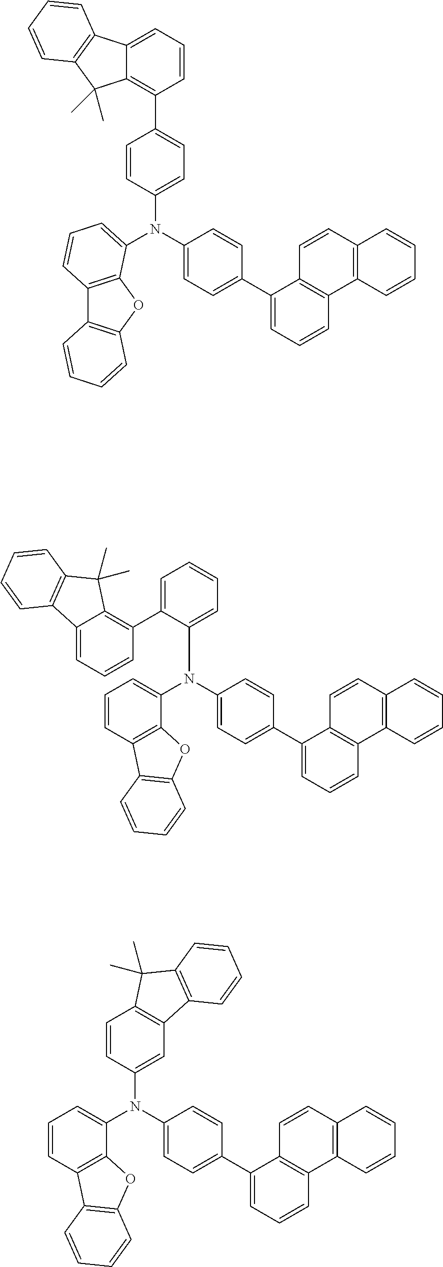 [SMILES: CC1(C)c2ccccc2-c2cccc(-c3ccc(N(c4ccc(-c5cccc6c5ccc5ccccc56)cc4)c4cccc5c4oc4ccccc45)cc3)c21 ; CC1(C)c2ccccc2-c2cccc(-c3ccccc3N(c3ccc(-c4cccc5c4ccc4ccccc45)cc3)c3cccc4c3oc3ccccc34)c21 ; CC1(C)c2ccccc2-c2cc(N(c3ccc(-c4cccc5c4ccc4ccccc45)cc3)c3cccc4c3oc3ccccc34)ccc21]

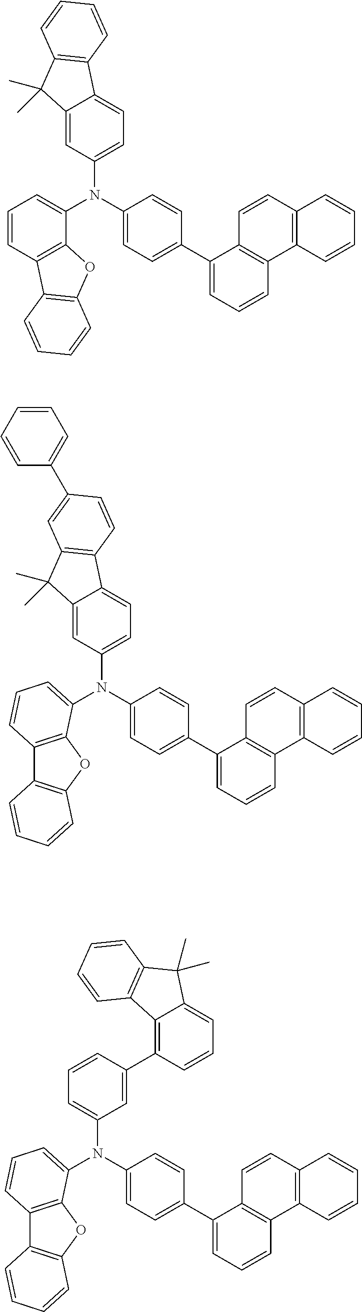 [SMILES: CC1(C)c2ccccc2-c2ccc(N(c3ccc(-c4cccc5c4ccc4ccccc45)cc3)c3cccc4c3oc3ccccc34)cc21 ; CC1(C)c2cc(-c3ccccc3)ccc2-c2ccc(N(c3ccc(-c4cccc5c4ccc4ccccc45)cc3)c3cccc4c3oc3ccccc34)cc21 ; CC1(C)c2ccccc2-c2c(-c3cccc(N(c4ccc(-c5cccc6c5ccc5ccccc56)cc4)c4cccc5c4oc4ccccc45)c3)cccc21]

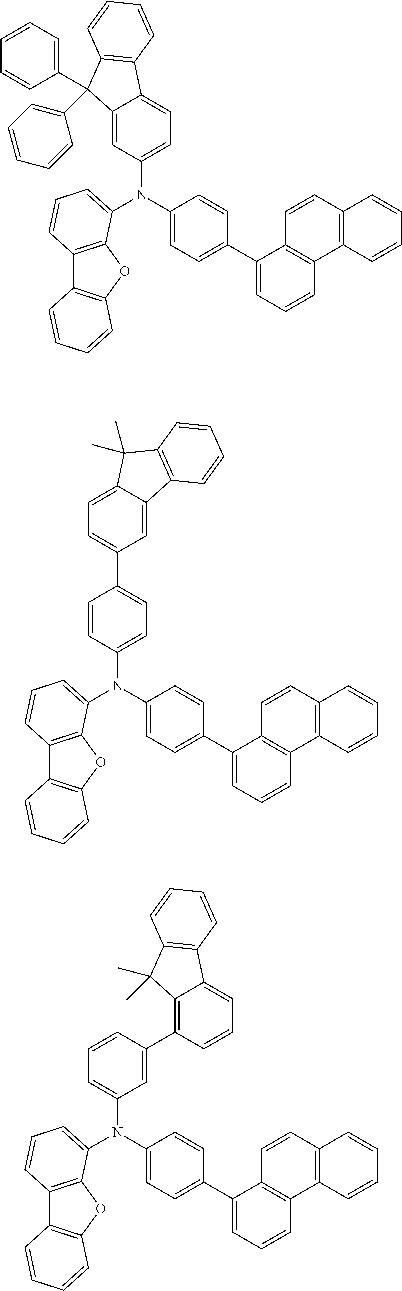 [SMILES: c1ccc(C2(c3ccccc3)c3ccccc3-c3ccc(N(c4ccc(-c5cccc6c5ccc5ccccc56)cc4)c4cccc5c4oc4ccccc45)cc32)cc1 ; CC1(C)c2ccccc2-c2cc(-c3ccc(N(c4ccc(-c5cccc6c5ccc5ccccc56)cc4)c4cccc5c4oc4ccccc45)cc3)ccc21 ; CC1(C)c2ccccc2-c2cccc(-c3cccc(N(c4ccc(-c5cccc6c5ccc5ccccc56)cc4)c4cccc5c4oc4ccccc45)c3)c21]

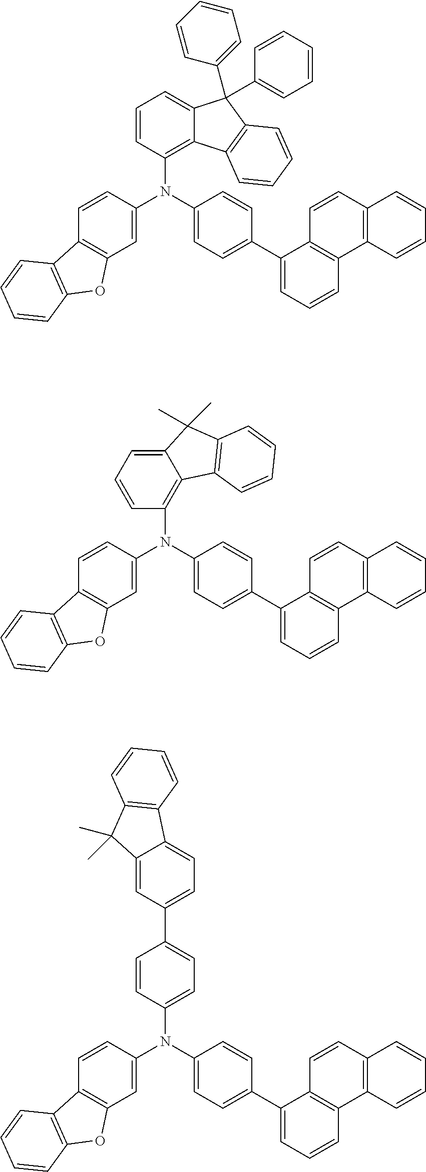 [SMILES: c1ccc(C2(c3ccccc3)c3ccccc3-c3c(N(c4ccc(-c5cccc6c5ccc5ccccc56)cc4)c4ccc5c(c4)oc4ccccc45)cccc32)cc1 ; CC1(C)c2ccccc2-c2c(N(c3ccc(-c4cccc5c4ccc4ccccc45)cc3)c3ccc4c(c3)oc3ccccc34)cccc21 ; CC1(C)c2ccccc2-c2ccc(-c3ccc(N(c4ccc(-c5cccc6c5ccc5ccccc56)cc4)c4ccc5c(c4)oc4ccccc45)cc3)cc21]

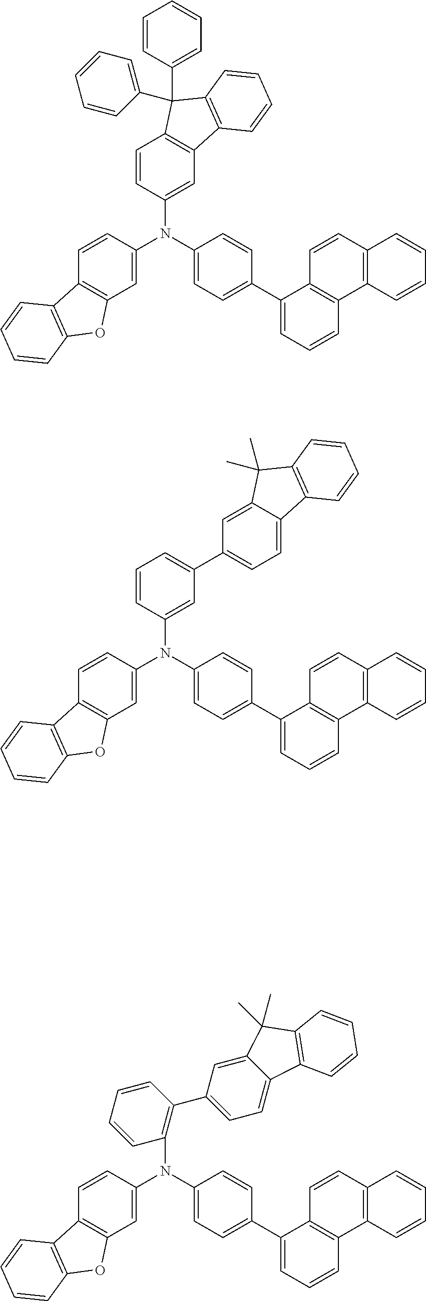 [SMILES: c1ccc(C2(c3ccccc3)c3ccccc3-c3cc(N(c4ccc(-c5cccc6c5ccc5ccccc56)cc4)c4ccc5c(c4)oc4ccccc45)ccc32)cc1 ; CC1(C)c2ccccc2-c2ccc(-c3cccc(N(c4ccc(-c5cccc6c5ccc5ccccc56)cc4)c4ccc5c(c4)oc4ccccc45)c3)cc21 ; CC1(C)c2ccccc2-c2ccc(-c3ccccc3N(c3ccc(-c4cccc5c4ccc4ccccc45)cc3)c3ccc4c(c3)oc3ccccc34)cc21]

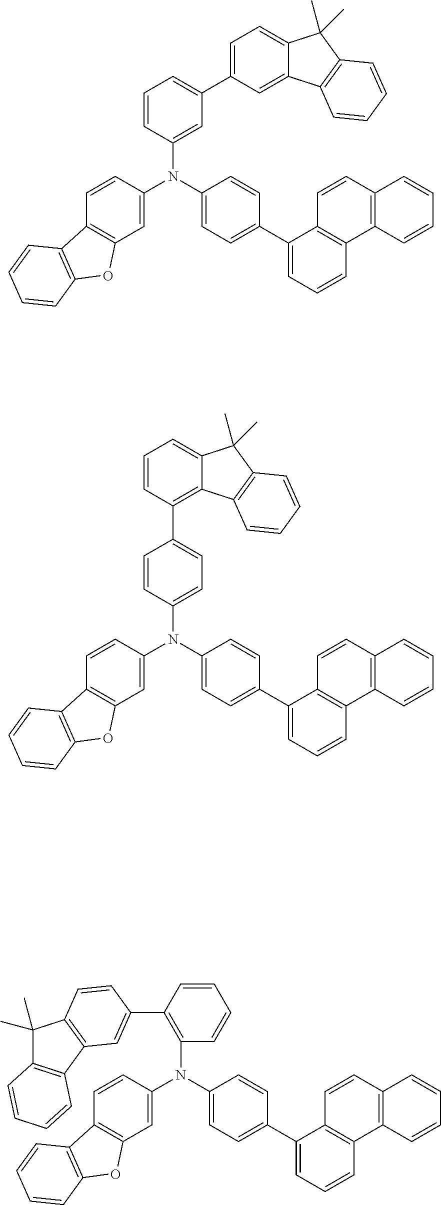 [SMILES: CC1(C)c2ccccc2-c2cc(-c3cccc(N(c4ccc(-c5cccc6c5ccc5ccccc56)cc4)c4ccc5c(c4)oc4ccccc45)c3)ccc21 ; CC1(C)c2ccccc2-c2c(-c3ccc(N(c4ccc(-c5cccc6c5ccc5ccccc56)cc4)c4ccc5c(c4)oc4ccccc45)cc3)cccc21 ; CC1(C)c2ccccc2-c2cc(-c3ccccc3N(c3ccc(-c4cccc5c4ccc4ccccc45)cc3)c3ccc4c(c3)oc3ccccc34)ccc21]

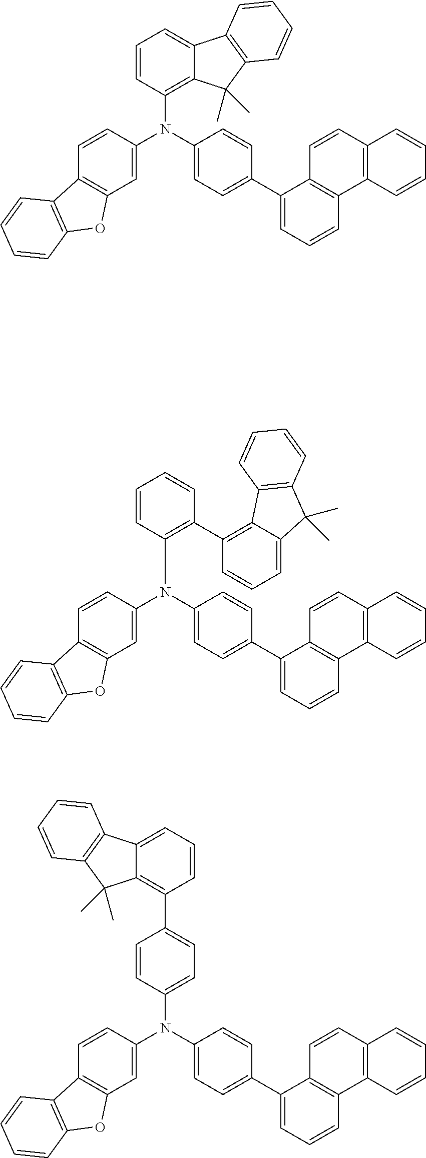 [SMILES: CC1(C)c2ccccc2-c2cccc(N(c3ccc(-c4cccc5c4ccc4ccccc45)cc3)c3ccc4c(c3)oc3ccccc34)c21 ; CC1(C)c2ccccc2-c2c(-c3ccccc3N(c3ccc(-c4cccc5c4ccc4ccccc45)cc3)c3ccc4c(c3)oc3ccccc34)cccc21 ; CC1(C)c2ccccc2-c2cccc(-c3ccc(N(c4ccc(-c5cccc6c5ccc5ccccc56)cc4)c4ccc5c(c4)oc4ccccc45)cc3)c21]

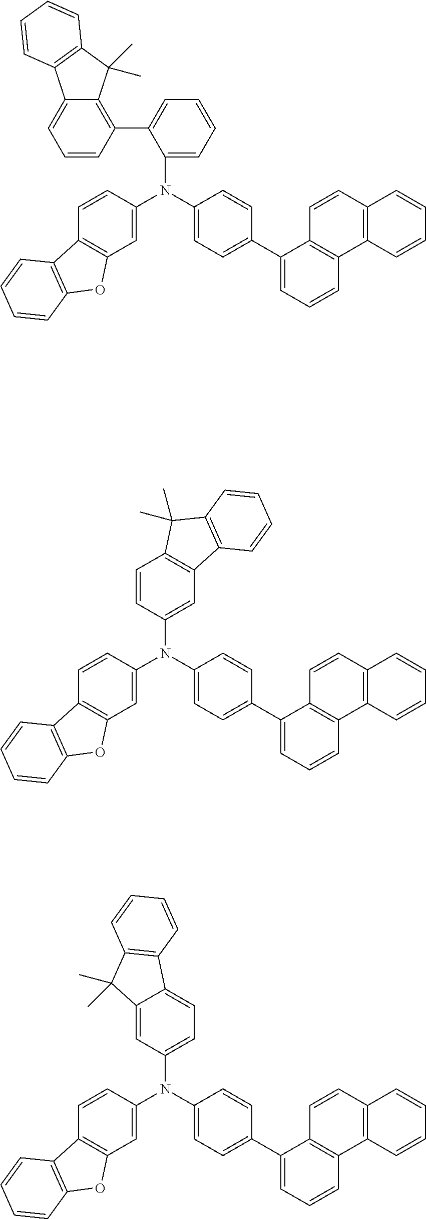 [SMILES: CC1(C)c2ccccc2-c2cccc(-c3ccccc3N(c3ccc(-c4cccc5c4ccc4ccccc45)cc3)c3ccc4c(c3)oc3ccccc34)c21 ; CC1(C)c2ccccc2-c2cc(N(c3ccc(-c4cccc5c4ccc4ccccc45)cc3)c3ccc4c(c3)oc3ccccc34)ccc21 ; CC1(C)c2ccccc2-c2ccc(N(c3ccc(-c4cccc5c4ccc4ccccc45)cc3)c3ccc4c(c3)oc3ccccc34)cc21]

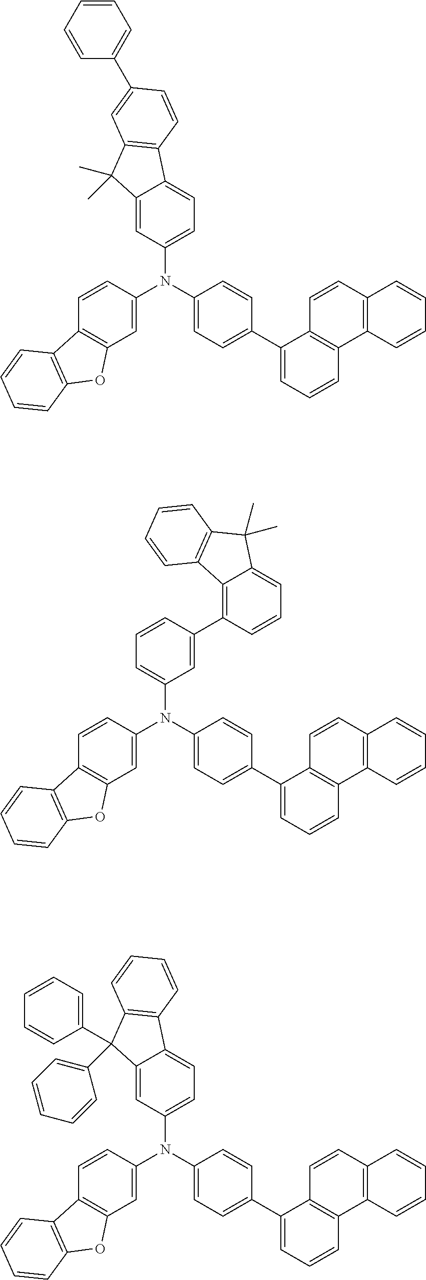 [SMILES: CC1(C)c2cc(-c3ccccc3)ccc2-c2ccc(N(c3ccc(-c4cccc5c4ccc4ccccc45)cc3)c3ccc4c(c3)oc3ccccc34)cc21 ; CC1(C)c2ccccc2-c2c(-c3cccc(N(c4ccc(-c5cccc6c5ccc5ccccc56)cc4)c4ccc5c(c4)oc4ccccc45)c3)cccc21 ; c1ccc(C2(c3ccccc3)c3ccccc3-c3ccc(N(c4ccc(-c5cccc6c5ccc5ccccc56)cc4)c4ccc5c(c4)oc4ccccc45)cc32)cc1]

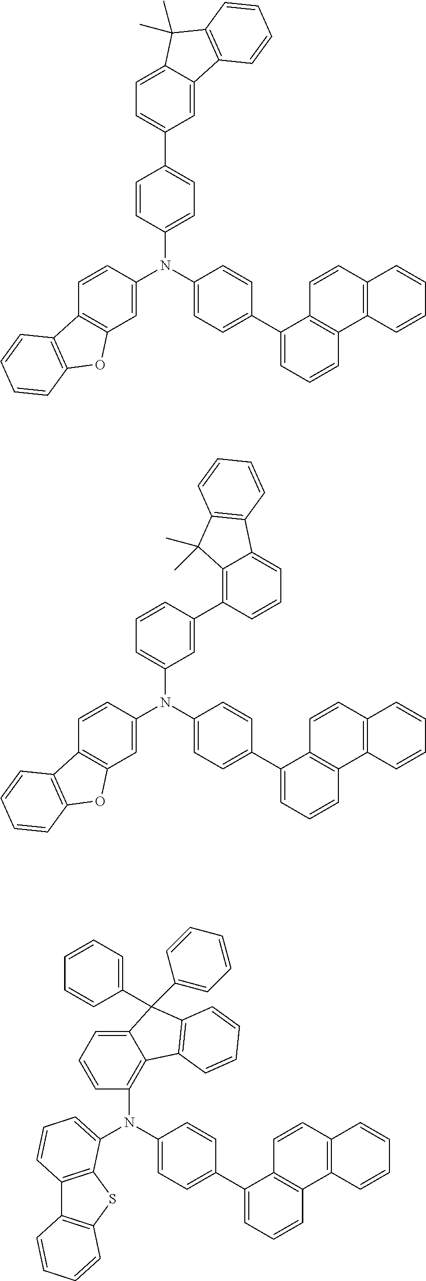 [SMILES: CC1(C)c2ccccc2-c2cc(-c3ccc(N(c4ccc(-c5cccc6c5ccc5ccccc56)cc4)c4ccc5c(c4)oc4ccccc45)cc3)ccc21 ; CC1(C)c2ccccc2-c2cccc(-c3cccc(N(c4ccc(-c5cccc6c5ccc5ccccc56)cc4)c4ccc5c(c4)oc4ccccc45)c3)c21 ; c1ccc(C2(c3ccccc3)c3ccccc3-c3c(N(c4ccc(-c5cccc6c5ccc5ccccc56)cc4)c4cccc5c4sc4ccccc45)cccc32)cc1]

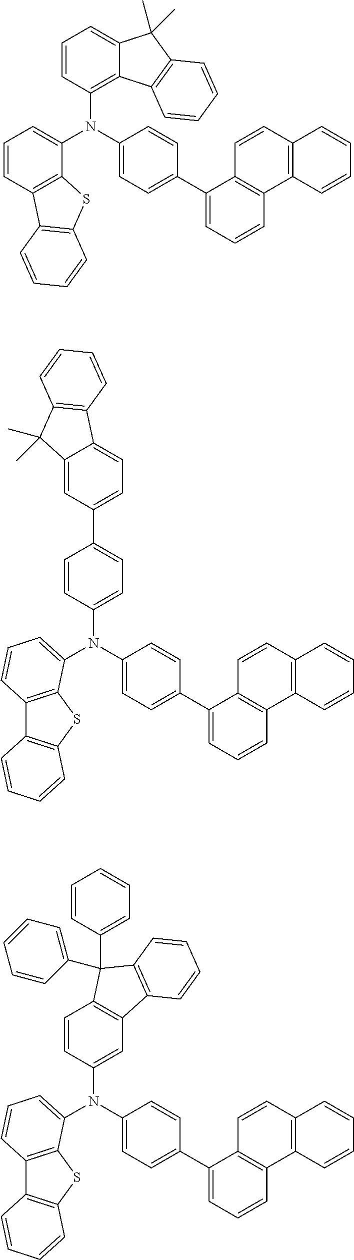 [SMILES: CC1(C)c2ccccc2-c2c(N(c3ccc(-c4cccc5c4ccc4ccccc45)cc3)c3cccc4c3sc3ccccc34)cccc21 ; CC1(C)c2ccccc2-c2ccc(-c3ccc(N(c4ccc(-c5cccc6c5ccc5ccccc56)cc4)c4cccc5c4sc4ccccc45)cc3)cc21 ; c1ccc(C2(c3ccccc3)c3ccccc3-c3cc(N(c4ccc(-c5cccc6c5ccc5ccccc56)cc4)c4cccc5c4sc4ccccc45)ccc32)cc1]

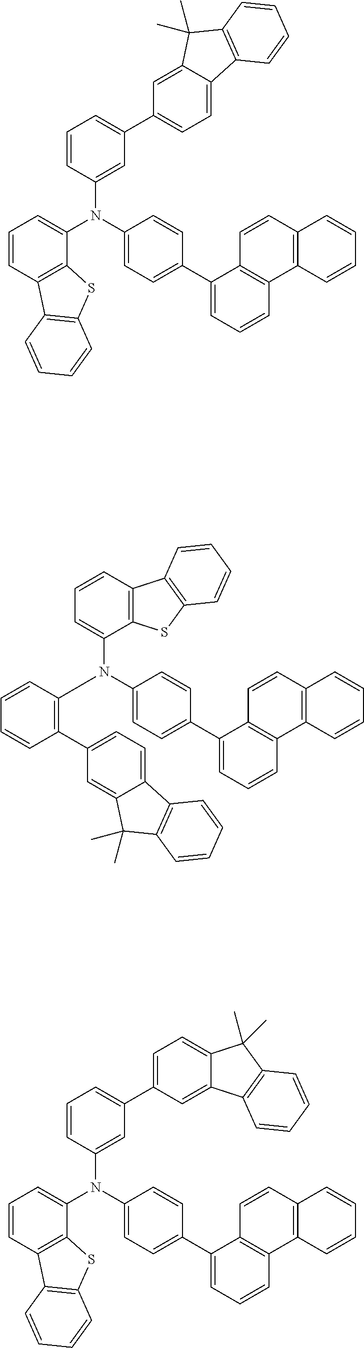 [SMILES: CC1(C)c2ccccc2-c2ccc(-c3cccc(N(c4ccc(-c5cccc6c5ccc5ccccc56)cc4)c4cccc5c4sc4ccccc45)c3)cc21 ; CC1(C)c2ccccc2-c2ccc(-c3ccccc3N(c3ccc(-c4cccc5c4ccc4ccccc45)cc3)c3cccc4c3sc3ccccc34)cc21 ; CC1(C)c2ccccc2-c2cc(-c3cccc(N(c4ccc(-c5cccc6c5ccc5ccccc56)cc4)c4cccc5c4sc4ccccc45)c3)ccc21]

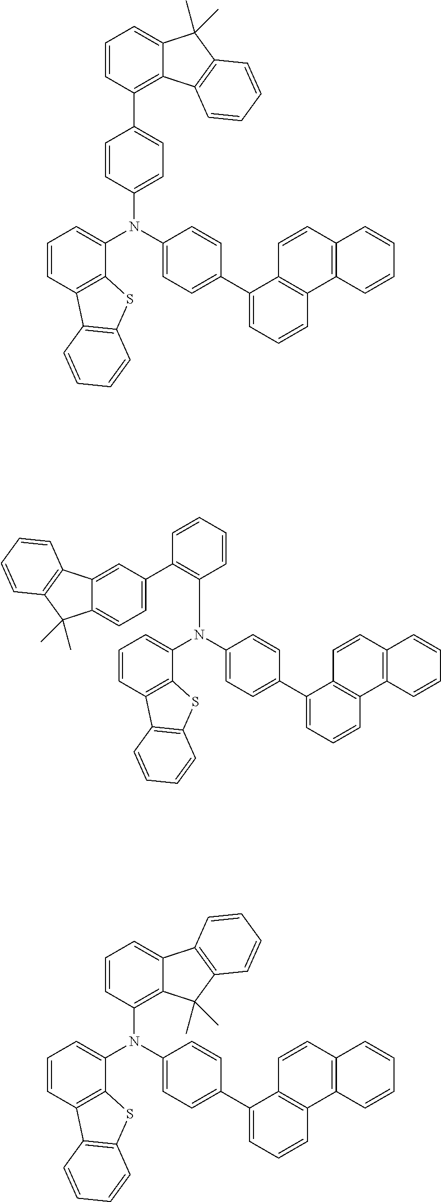 [SMILES: CC1(C)c2ccccc2-c2c(-c3ccc(N(c4ccc(-c5cccc6c5ccc5ccccc56)cc4)c4cccc5c4sc4ccccc45)cc3)cccc21 ; CC1(C)c2ccccc2-c2cc(-c3ccccc3N(c3ccc(-c4cccc5c4ccc4ccccc45)cc3)c3cccc4c3sc3ccccc34)ccc21 ; CC1(C)c2ccccc2-c2cccc(N(c3ccc(-c4cccc5c4ccc4ccccc45)cc3)c3cccc4c3sc3ccccc34)c21]

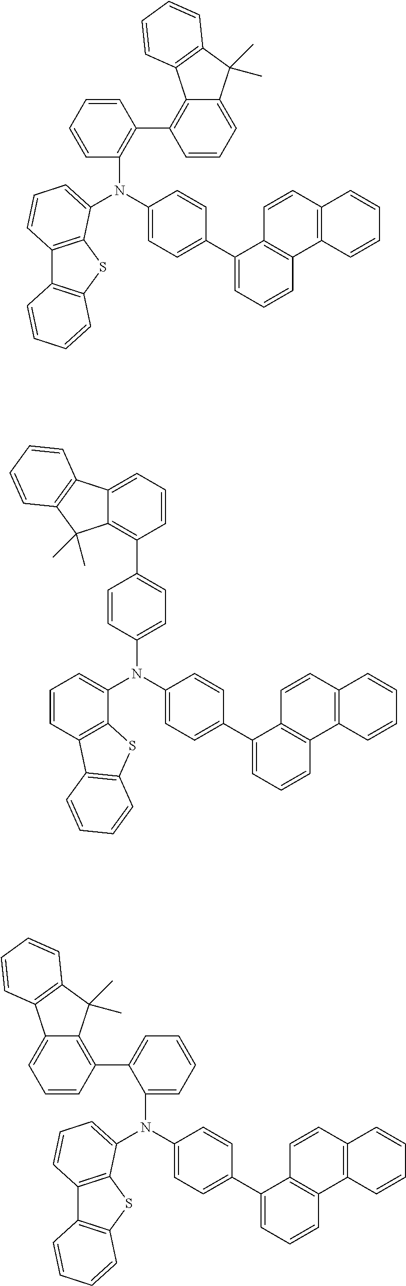 [SMILES: CC1(C)c2ccccc2-c2c(-c3ccccc3N(c3ccc(-c4cccc5c4ccc4ccccc45)cc3)c3cccc4c3sc3ccccc34)cccc21 ; CC1(C)c2ccccc2-c2cccc(-c3ccc(N(c4ccc(-c5cccc6c5ccc5ccccc56)cc4)c4cccc5c4sc4ccccc45)cc3)c21 ; CC1(C)c2ccccc2-c2cccc(-c3ccccc3N(c3ccc(-c4cccc5c4ccc4ccccc45)cc3)c3cccc4c3sc3ccccc34)c21]

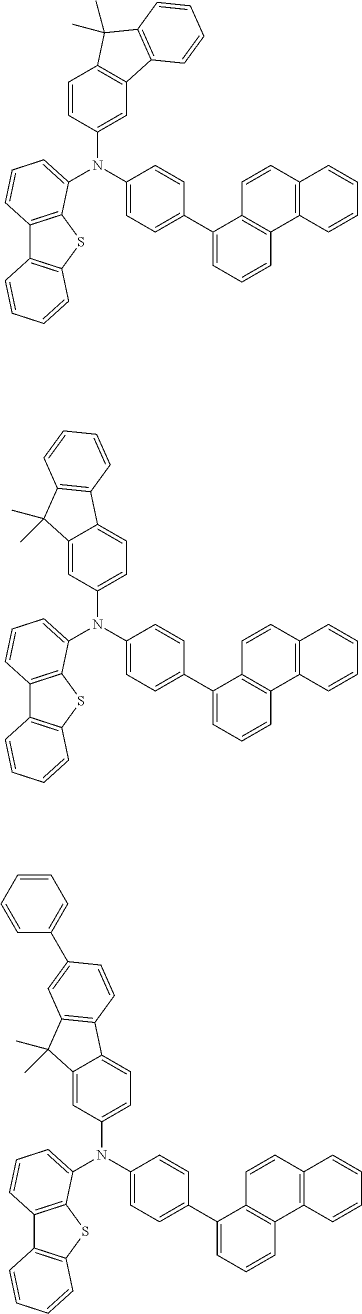 [SMILES: CC1(C)c2ccccc2-c2cc(N(c3ccc(-c4cccc5c4ccc4ccccc45)cc3)c3cccc4c3sc3ccccc34)ccc21 ; CC1(C)c2ccccc2-c2ccc(N(c3ccc(-c4cccc5c4ccc4ccccc45)cc3)c3cccc4c3sc3ccccc34)cc21 ; CC1(C)c2cc(-c3ccccc3)ccc2-c2ccc(N(c3ccc(-c4cccc5c4ccc4ccccc45)cc3)c3cccc4c3sc3ccccc34)cc21]

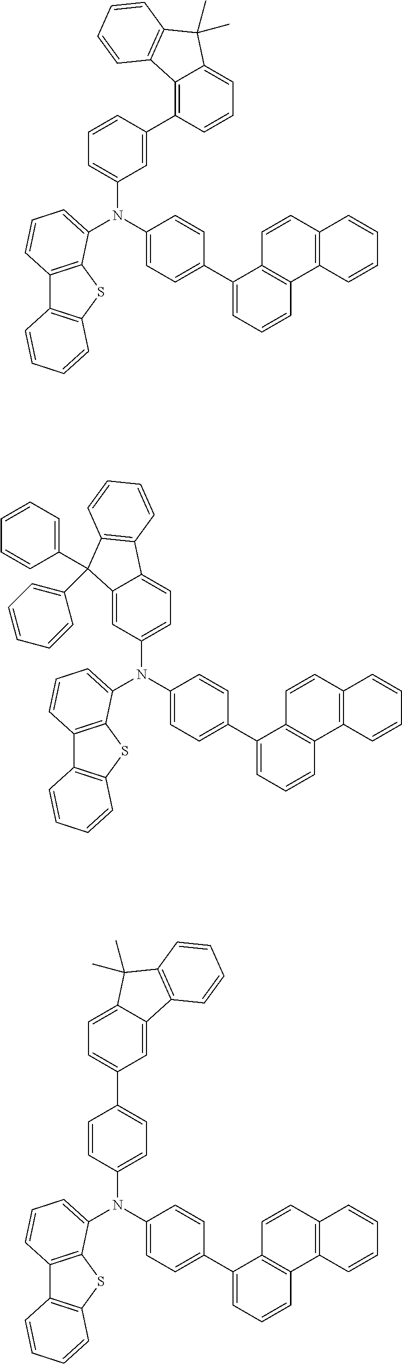 [SMILES: CC1(C)c2ccccc2-c2c(-c3cccc(N(c4ccc(-c5cccc6c5ccc5ccccc56)cc4)c4cccc5c4sc4ccccc45)c3)cccc21 ; c1ccc(C2(c3ccccc3)c3ccccc3-c3ccc(N(c4ccc(-c5cccc6c5ccc5ccccc56)cc4)c4cccc5c4sc4ccccc45)cc32)cc1 ; CC1(C)c2ccccc2-c2cc(-c3ccc(N(c4ccc(-c5cccc6c5ccc5ccccc56)cc4)c4cccc5c4sc4ccccc45)cc3)ccc21]

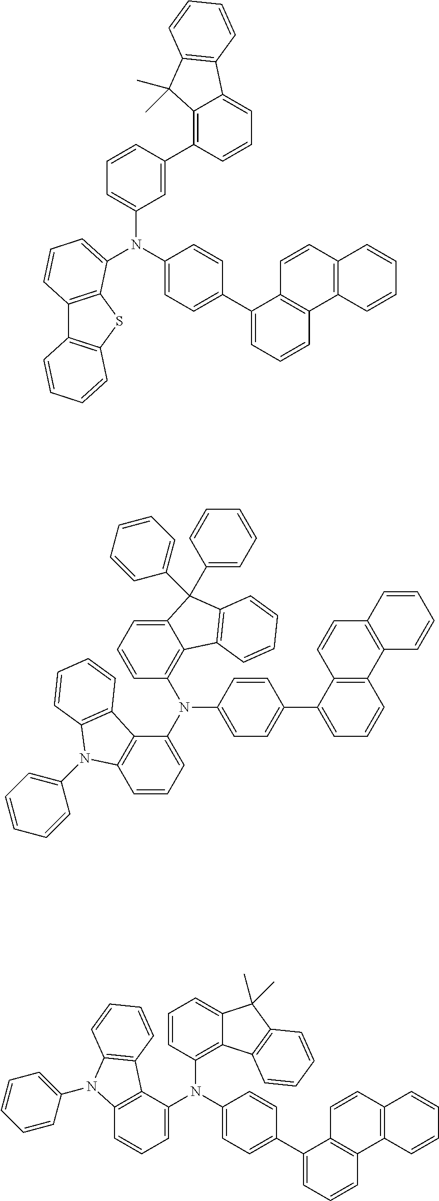 [SMILES: CC1(C)c2ccccc2-c2cccc(-c3cccc(N(c4ccc(-c5cccc6c5ccc5ccccc56)cc4)c4cccc5c4sc4ccccc45)c3)c21 ; c1ccc(-n2c3ccccc3c3c(N(c4ccc(-c5cccc6c5ccc5ccccc56)cc4)c4cccc5c4-c4ccccc4C5(c4ccccc4)c4ccccc4)cccc32)cc1 ; CC1(C)c2ccccc2-c2c(N(c3ccc(-c4cccc5c4ccc4ccccc45)cc3)c3cccc4c3c3ccccc3n4-c3ccccc3)cccc21]

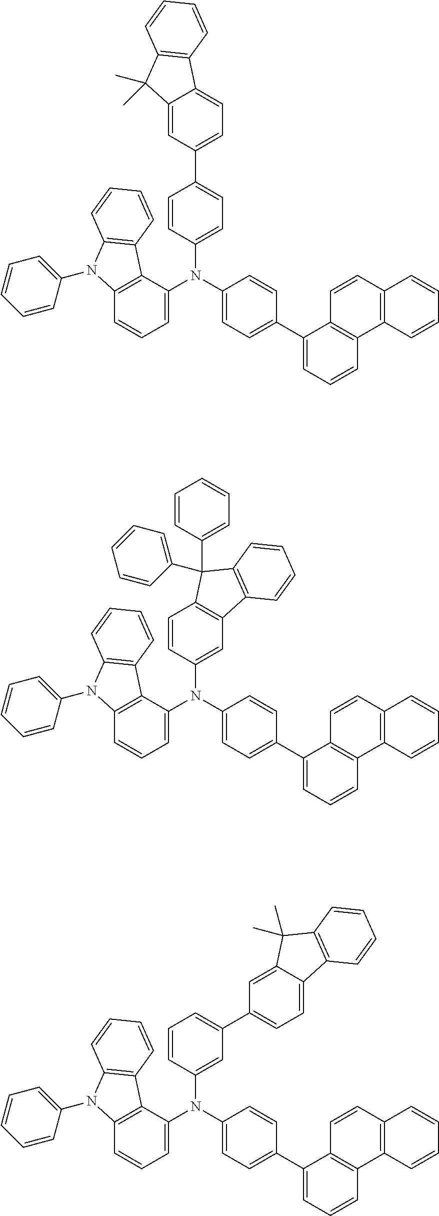 [SMILES: CC1(C)c2ccccc2-c2ccc(-c3ccc(N(c4ccc(-c5cccc6c5ccc5ccccc56)cc4)c4cccc5c4c4ccccc4n5-c4ccccc4)cc3)cc21 ; c1ccc(-n2c3ccccc3c3c(N(c4ccc(-c5cccc6c5ccc5ccccc56)cc4)c4ccc5c(c4)-c4ccccc4C5(c4ccccc4)c4ccccc4)cccc32)cc1 ; CC1(C)c2ccccc2-c2ccc(-c3cccc(N(c4ccc(-c5cccc6c5ccc5ccccc56)cc4)c4cccc5c4c4ccccc4n5-c4ccccc4)c3)cc21]

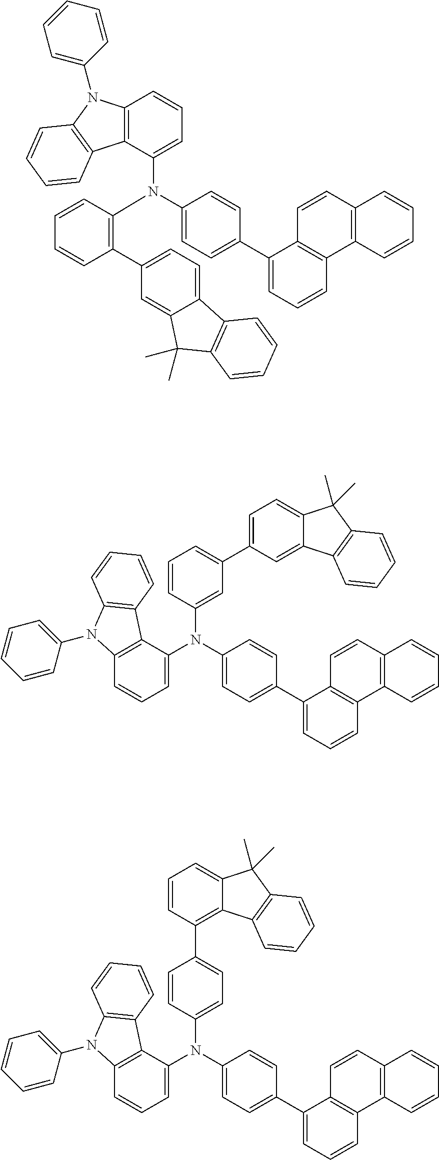 [SMILES: CC1(C)c2ccccc2-c2ccc(-c3ccccc3N(c3ccc(-c4cccc5c4ccc4ccccc45)cc3)c3cccc4c3c3ccccc3n4-c3ccccc3)cc21 ; CC1(C)c2ccccc2-c2cc(-c3cccc(N(c4ccc(-c5cccc6c5ccc5ccccc56)cc4)c4cccc5c4c4ccccc4n5-c4ccccc4)c3)ccc21 ; CC1(C)c2ccccc2-c2c(-c3ccc(N(c4ccc(-c5cccc6c5ccc5ccccc56)cc4)c4cccc5c4c4ccccc4n5-c4ccccc4)cc3)cccc21]

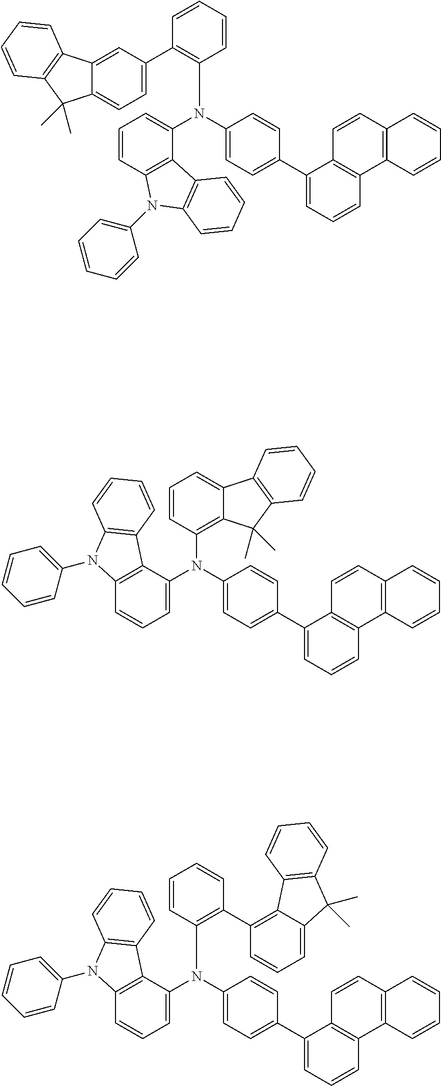 [SMILES: CC1(C)c2ccccc2-c2cc(-c3ccccc3N(c3ccc(-c4cccc5c4ccc4ccccc45)cc3)c3cccc4c3c3ccccc3n4-c3ccccc3)ccc21 ; CC1(C)c2ccccc2-c2cccc(N(c3ccc(-c4cccc5c4ccc4ccccc45)cc3)c3cccc4c3c3ccccc3n4-c3ccccc3)c21 ; CC1(C)c2ccccc2-c2c(-c3ccccc3N(c3ccc(-c4cccc5c4ccc4ccccc45)cc3)c3cccc4c3c3ccccc3n4-c3ccccc3)cccc21]

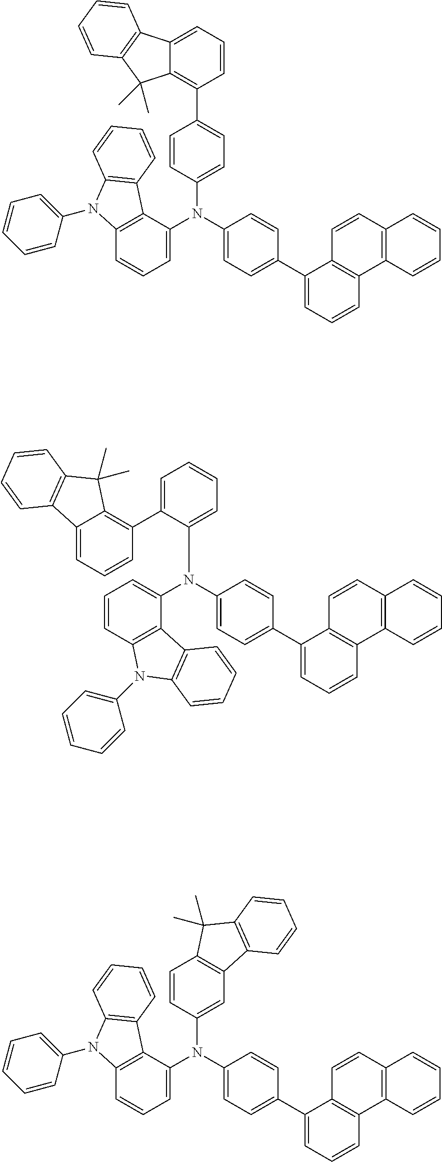 [SMILES: CC1(C)c2ccccc2-c2cccc(-c3ccc(N(c4ccc(-c5cccc6c5ccc5ccccc56)cc4)c4cccc5c4c4ccccc4n5-c4ccccc4)cc3)c21 ; CC1(C)c2ccccc2-c2cccc(-c3ccccc3N(c3ccc(-c4cccc5c4ccc4ccccc45)cc3)c3cccc4c3c3ccccc3n4-c3ccccc3)c21 ; CC1(C)c2ccccc2-c2cc(N(c3ccc(-c4cccc5c4ccc4ccccc45)cc3)c3cccc4c3c3ccccc3n4-c3ccccc3)ccc21]

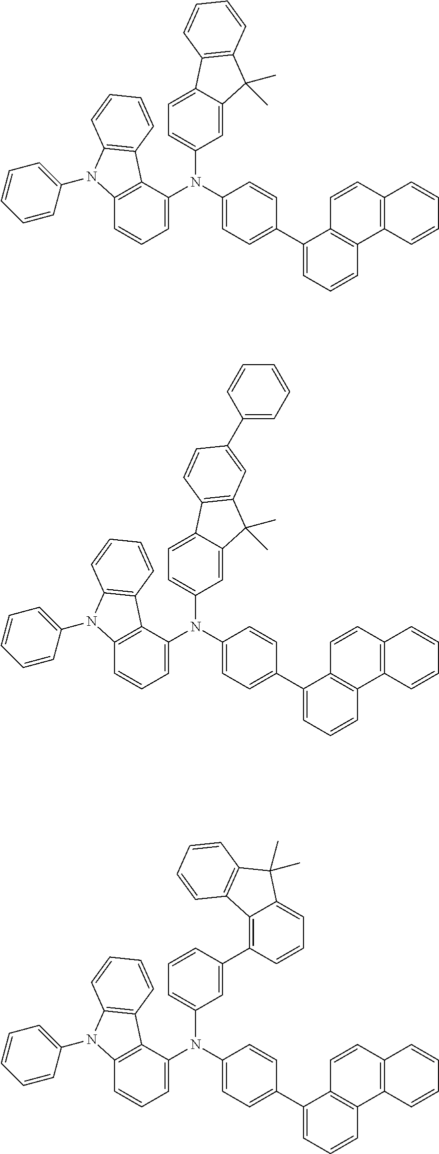 [SMILES: CC1(C)c2ccccc2-c2ccc(N(c3ccc(-c4cccc5c4ccc4ccccc45)cc3)c3cccc4c3c3ccccc3n4-c3ccccc3)cc21 ; CC1(C)c2cc(-c3ccccc3)ccc2-c2ccc(N(c3ccc(-c4cccc5c4ccc4ccccc45)cc3)c3cccc4c3c3ccccc3n4-c3ccccc3)cc21 ; CC1(C)c2ccccc2-c2c(-c3cccc(N(c4ccc(-c5cccc6c5ccc5ccccc56)cc4)c4cccc5c4c4ccccc4n5-c4ccccc4)c3)cccc21]

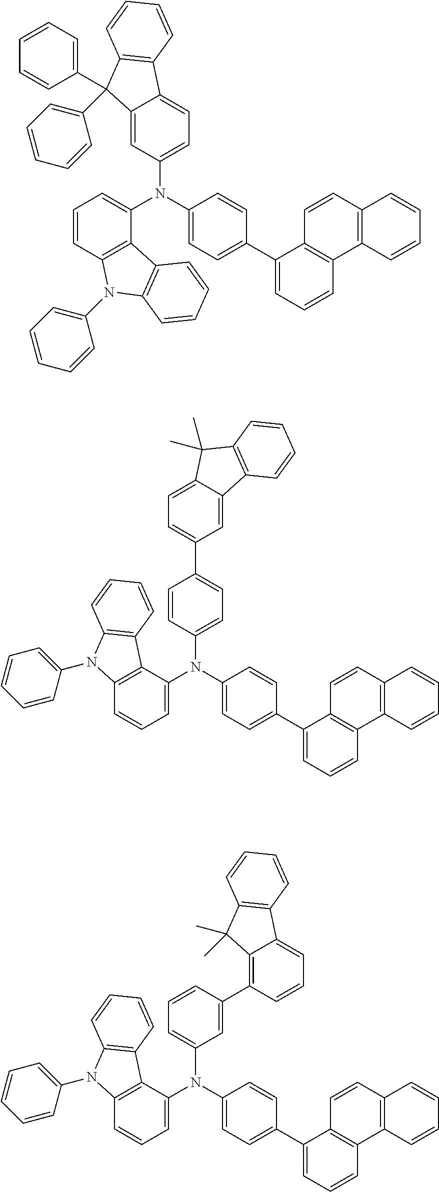 [SMILES: c1ccc(-n2c3ccccc3c3c(N(c4ccc(-c5cccc6c5ccc5ccccc56)cc4)c4ccc5c(c4)C(c4ccccc4)(c4ccccc4)c4ccccc4-5)cccc32)cc1 ; CC1(C)c2ccccc2-c2cc(-c3ccc(N(c4ccc(-c5cccc6c5ccc5ccccc56)cc4)c4cccc5c4c4ccccc4n5-c4ccccc4)cc3)ccc21 ; CC1(C)c2ccccc2-c2cccc(-c3cccc(N(c4ccc(-c5cccc6c5ccc5ccccc56)cc4)c4cccc5c4c4ccccc4n5-c4ccccc4)c3)c21]

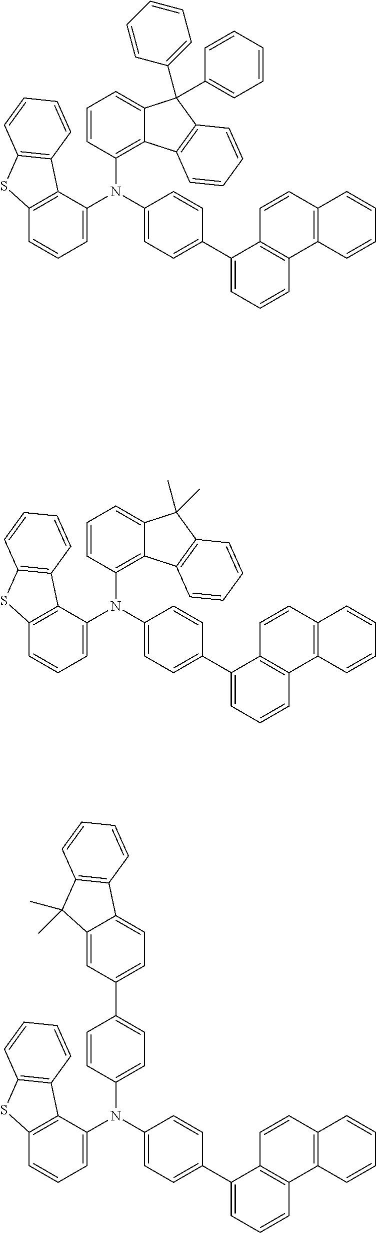 [SMILES: c1ccc(C2(c3ccccc3)c3ccccc3-c3c(N(c4ccc(-c5cccc6c5ccc5ccccc56)cc4)c4cccc5sc6ccccc6c45)cccc32)cc1 ; CC1(C)c2ccccc2-c2c(N(c3ccc(-c4cccc5c4ccc4ccccc45)cc3)c3cccc4sc5ccccc5c34)cccc21 ; CC1(C)c2ccccc2-c2ccc(-c3ccc(N(c4ccc(-c5cccc6c5ccc5ccccc56)cc4)c4cccc5sc6ccccc6c45)cc3)cc21]

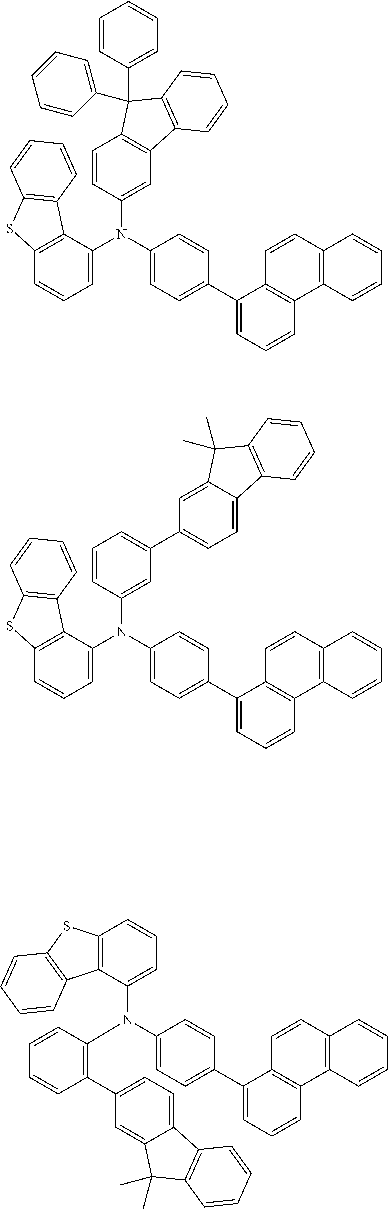 [SMILES: c1ccc(C2(c3ccccc3)c3ccccc3-c3cc(N(c4ccc(-c5cccc6c5ccc5ccccc56)cc4)c4cccc5sc6ccccc6c45)ccc32)cc1 ; CC1(C)c2ccccc2-c2ccc(-c3cccc(N(c4ccc(-c5cccc6c5ccc5ccccc56)cc4)c4cccc5sc6ccccc6c45)c3)cc21 ; CC1(C)c2ccccc2-c2ccc(-c3ccccc3N(c3ccc(-c4cccc5c4ccc4ccccc45)cc3)c3cccc4sc5ccccc5c34)cc21]

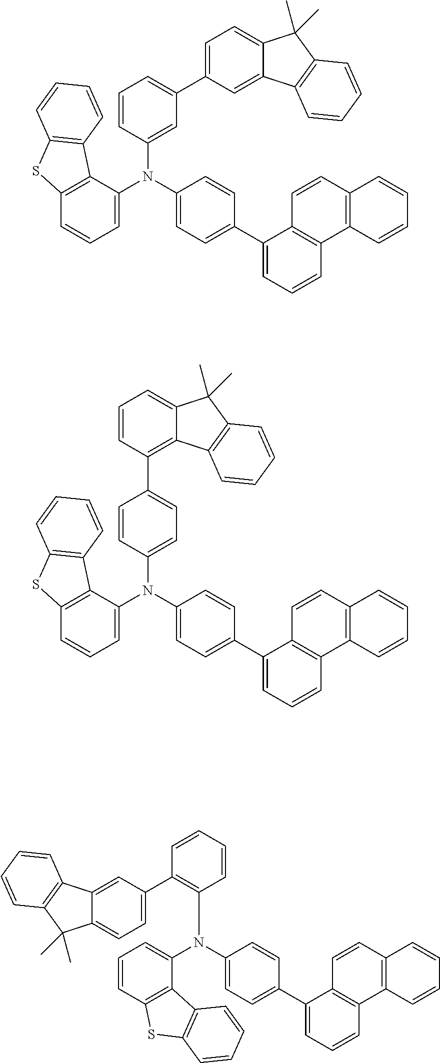 [SMILES: CC1(C)c2ccccc2-c2cc(-c3cccc(N(c4ccc(-c5cccc6c5ccc5ccccc56)cc4)c4cccc5sc6ccccc6c45)c3)ccc21 ; CC1(C)c2ccccc2-c2c(-c3ccc(N(c4ccc(-c5cccc6c5ccc5ccccc56)cc4)c4cccc5sc6ccccc6c45)cc3)cccc21 ; CC1(C)c2ccccc2-c2cc(-c3ccccc3N(c3ccc(-c4cccc5c4ccc4ccccc45)cc3)c3cccc4sc5ccccc5c34)ccc21]

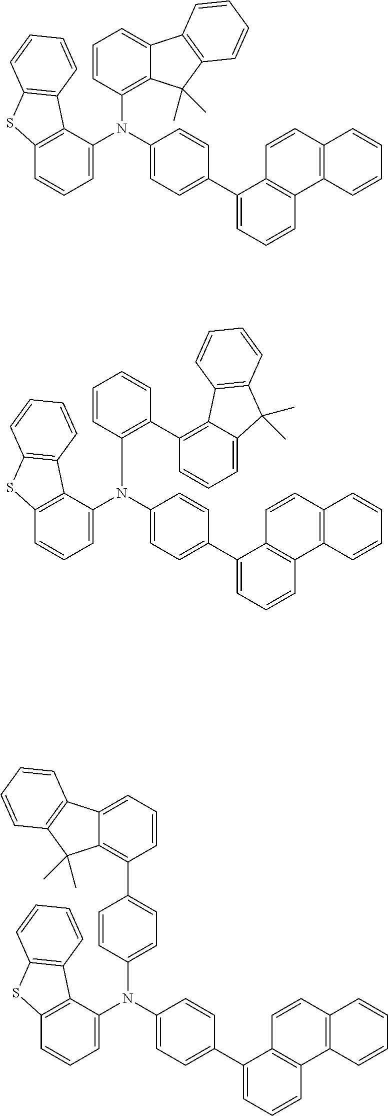 [SMILES: CC1(C)c2ccccc2-c2cccc(N(c3ccc(-c4cccc5c4ccc4ccccc45)cc3)c3cccc4sc5ccccc5c34)c21 ; CC1(C)c2ccccc2-c2c(-c3ccccc3N(c3ccc(-c4cccc5c4ccc4ccccc45)cc3)c3cccc4sc5ccccc5c34)cccc21 ; CC1(C)c2ccccc2-c2cccc(-c3ccc(N(c4ccc(-c5cccc6c5ccc5ccccc56)cc4)c4cccc5sc6ccccc6c45)cc3)c21]

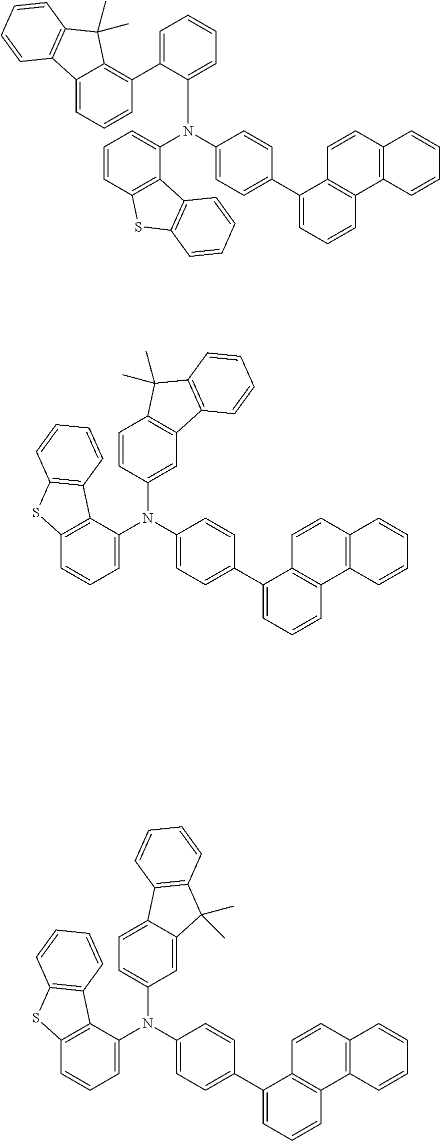 [SMILES: CC1(C)c2ccccc2-c2cccc(-c3ccccc3N(c3ccc(-c4cccc5c4ccc4ccccc45)cc3)c3cccc4sc5ccccc5c34)c21 ; CC1(C)c2ccccc2-c2cc(N(c3ccc(-c4cccc5c4ccc4ccccc45)cc3)c3cccc4sc5ccccc5c34)ccc21 ; CC1(C)c2ccccc2-c2ccc(N(c3ccc(-c4cccc5c4ccc4ccccc45)cc3)c3cccc4sc5ccccc5c34)cc21]

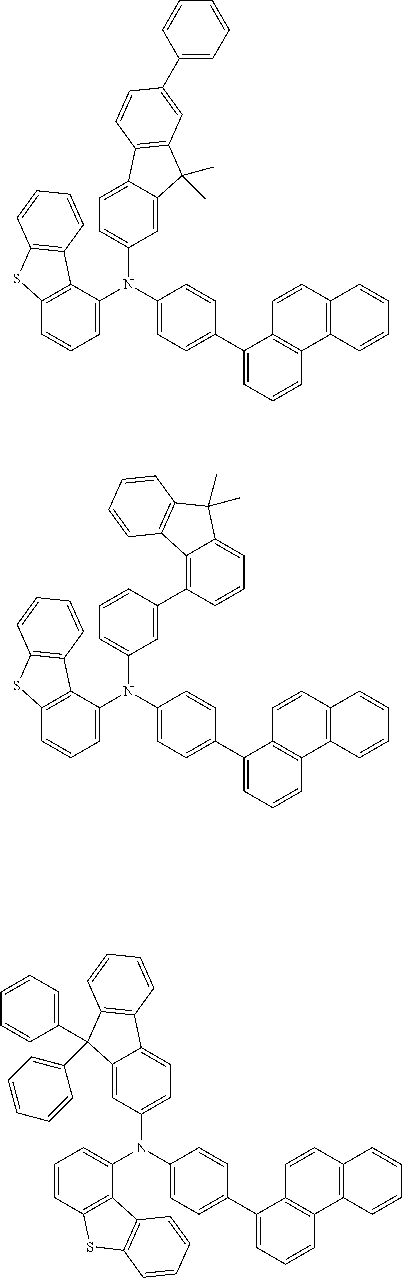 [SMILES: CC1(C)c2cc(-c3ccccc3)ccc2-c2ccc(N(c3ccc(-c4cccc5c4ccc4ccccc45)cc3)c3cccc4sc5ccccc5c34)cc21 ; CC1(C)c2ccccc2-c2c(-c3cccc(N(c4ccc(-c5cccc6c5ccc5ccccc56)cc4)c4cccc5sc6ccccc6c45)c3)cccc21 ; c1ccc(C2(c3ccccc3)c3ccccc3-c3ccc(N(c4ccc(-c5cccc6c5ccc5ccccc56)cc4)c4cccc5sc6ccccc6c45)cc32)cc1]

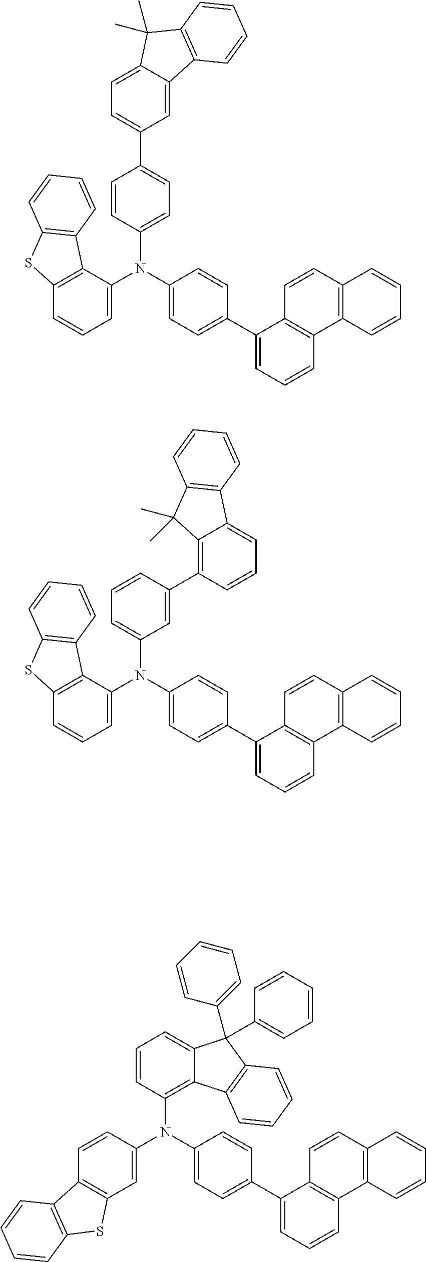 [SMILES: CC1(C)c2ccccc2-c2cc(-c3ccc(N(c4ccc(-c5cccc6c5ccc5ccccc56)cc4)c4cccc5sc6ccccc6c45)cc3)ccc21 ; CC1(C)c2ccccc2-c2cccc(-c3cccc(N(c4ccc(-c5cccc6c5ccc5ccccc56)cc4)c4cccc5sc6ccccc6c45)c3)c21 ; c1ccc(C2(c3ccccc3)c3ccccc3-c3c(N(c4ccc(-c5cccc6c5ccc5ccccc56)cc4)c4ccc5c(c4)sc4ccccc45)cccc32)cc1]

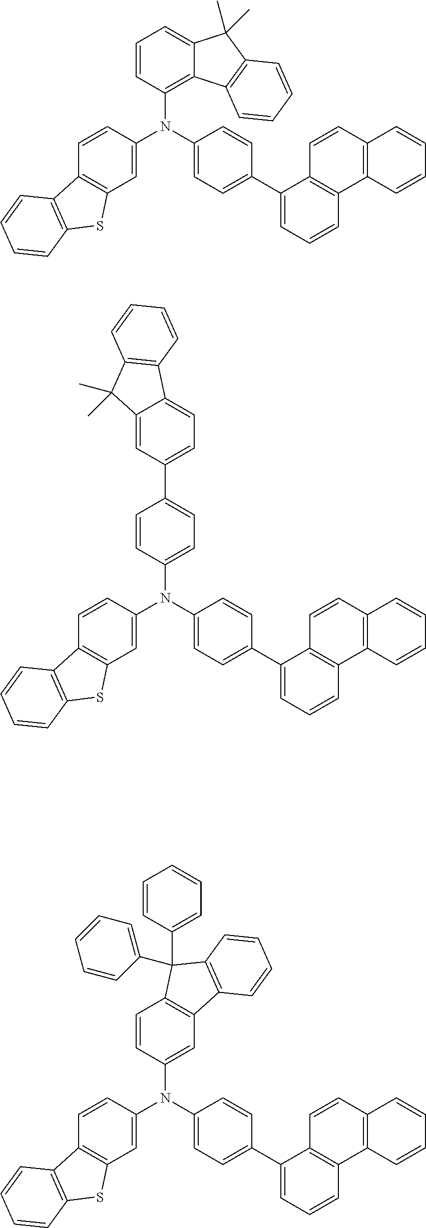 [SMILES: CC1(C)c2ccccc2-c2c(N(c3ccc(-c4cccc5c4ccc4ccccc45)cc3)c3ccc4c(c3)sc3ccccc34)cccc21 ; CC1(C)c2ccccc2-c2ccc(-c3ccc(N(c4ccc(-c5cccc6c5ccc5ccccc56)cc4)c4ccc5c(c4)sc4ccccc45)cc3)cc21 ; c1ccc(C2(c3ccccc3)c3ccccc3-c3cc(N(c4ccc(-c5cccc6c5ccc5ccccc56)cc4)c4ccc5c(c4)sc4ccccc45)ccc32)cc1]

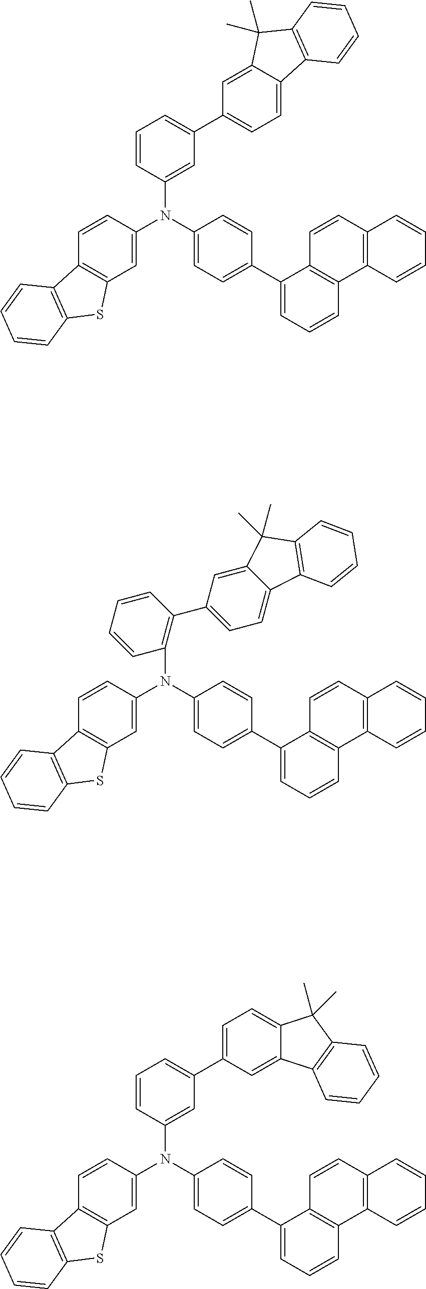 [SMILES: CC1(C)c2ccccc2-c2ccc(-c3cccc(N(c4ccc(-c5cccc6c5ccc5ccccc56)cc4)c4ccc5c(c4)sc4ccccc45)c3)cc21 ; CC1(C)c2ccccc2-c2ccc(-c3ccccc3N(c3ccc(-c4cccc5c4ccc4ccccc45)cc3)c3ccc4c(c3)sc3ccccc34)cc21 ; CC1(C)c2ccccc2-c2cc(-c3cccc(N(c4ccc(-c5cccc6c5ccc5ccccc56)cc4)c4ccc5c(c4)sc4ccccc45)c3)ccc21]

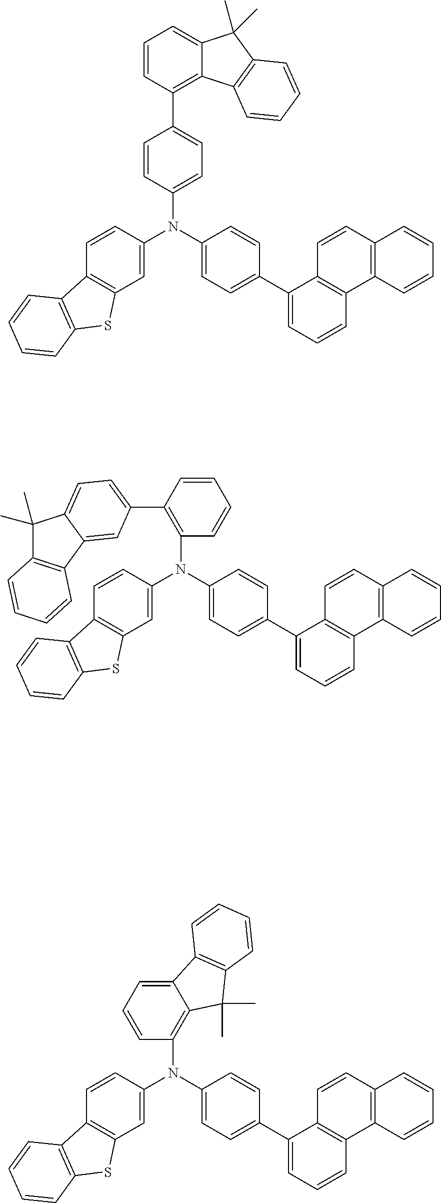 [SMILES: CC1(C)c2ccccc2-c2c(-c3ccc(N(c4ccc(-c5cccc6c5ccc5ccccc56)cc4)c4ccc5c(c4)sc4ccccc45)cc3)cccc21 ; CC1(C)c2ccccc2-c2cc(-c3ccccc3N(c3ccc(-c4cccc5c4ccc4ccccc45)cc3)c3ccc4c(c3)sc3ccccc34)ccc21 ; CC1(C)c2ccccc2-c2cccc(N(c3ccc(-c4cccc5c4ccc4ccccc45)cc3)c3ccc4c(c3)sc3ccccc34)c21]

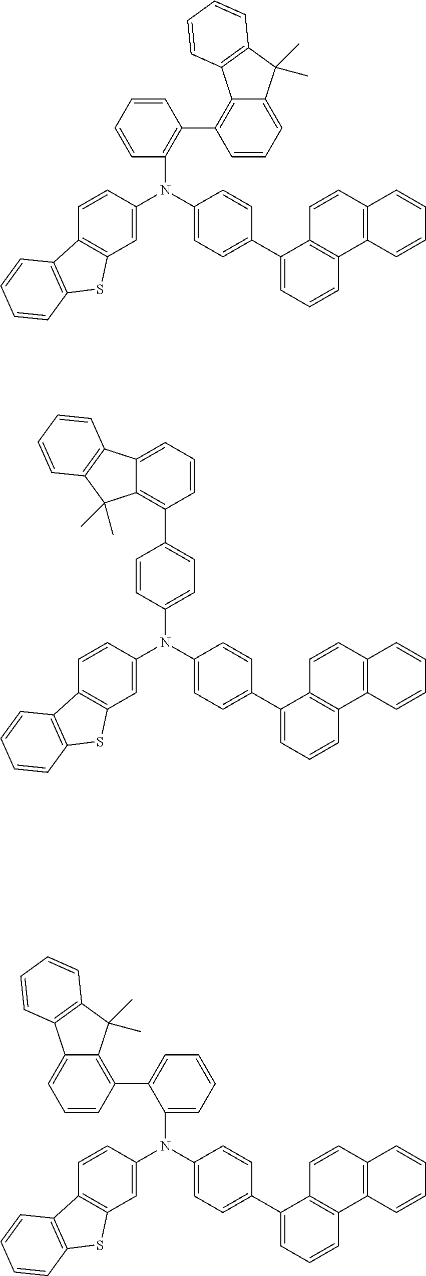 [SMILES: CC1(C)c2ccccc2-c2c(-c3ccccc3N(c3ccc(-c4cccc5c4ccc4ccccc45)cc3)c3ccc4c(c3)sc3ccccc34)cccc21 ; CC1(C)c2ccccc2-c2cccc(-c3ccc(N(c4ccc(-c5cccc6c5ccc5ccccc56)cc4)c4ccc5c(c4)sc4ccccc45)cc3)c21 ; CC1(C)c2ccccc2-c2cccc(-c3ccccc3N(c3ccc(-c4cccc5c4ccc4ccccc45)cc3)c3ccc4c(c3)sc3ccccc34)c21]

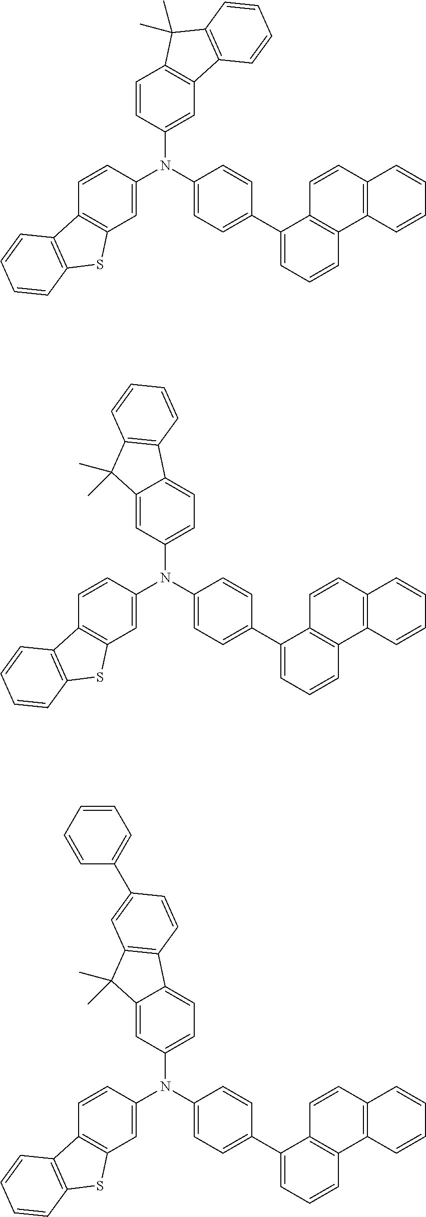 [SMILES: CC1(C)c2ccccc2-c2cc(N(c3ccc(-c4cccc5c4ccc4ccccc45)cc3)c3ccc4c(c3)sc3ccccc34)ccc21 ; CC1(C)c2ccccc2-c2ccc(N(c3ccc(-c4cccc5c4ccc4ccccc45)cc3)c3ccc4c(c3)sc3ccccc34)cc21 ; CC1(C)c2cc(-c3ccccc3)ccc2-c2ccc(N(c3ccc(-c4cccc5c4ccc4ccccc45)cc3)c3ccc4c(c3)sc3ccccc34)cc21]

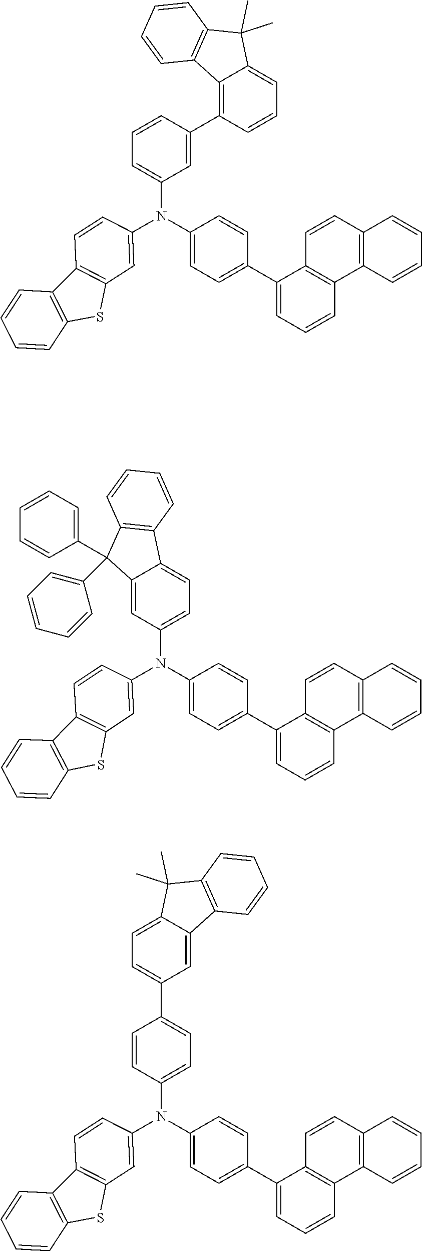 [SMILES: CC1(C)c2ccccc2-c2c(-c3cccc(N(c4ccc(-c5cccc6c5ccc5ccccc56)cc4)c4ccc5c(c4)sc4ccccc45)c3)cccc21 ; c1ccc(C2(c3ccccc3)c3ccccc3-c3ccc(N(c4ccc(-c5cccc6c5ccc5ccccc56)cc4)c4ccc5c(c4)sc4ccccc45)cc32)cc1 ; CC1(C)c2ccccc2-c2cc(-c3ccc(N(c4ccc(-c5cccc6c5ccc5ccccc56)cc4)c4ccc5c(c4)sc4ccccc45)cc3)ccc21]

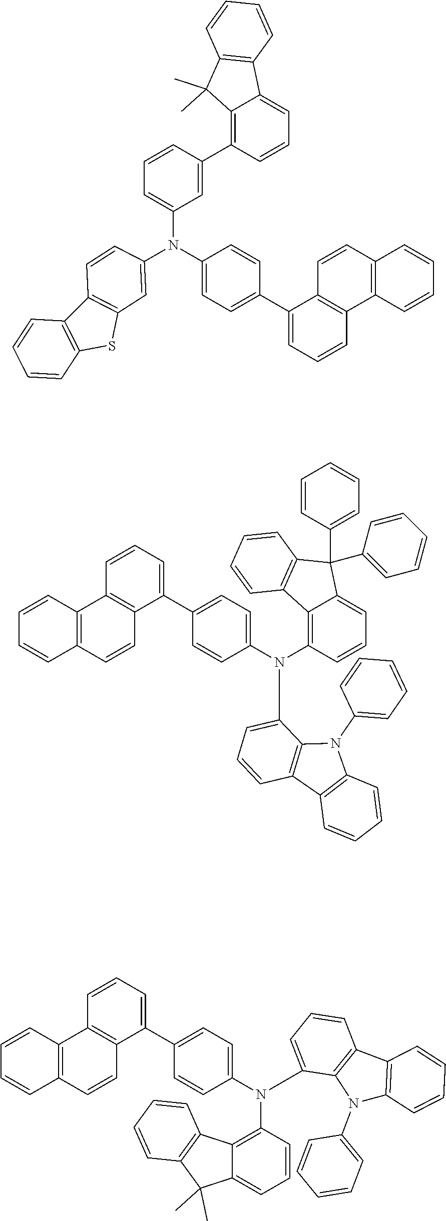

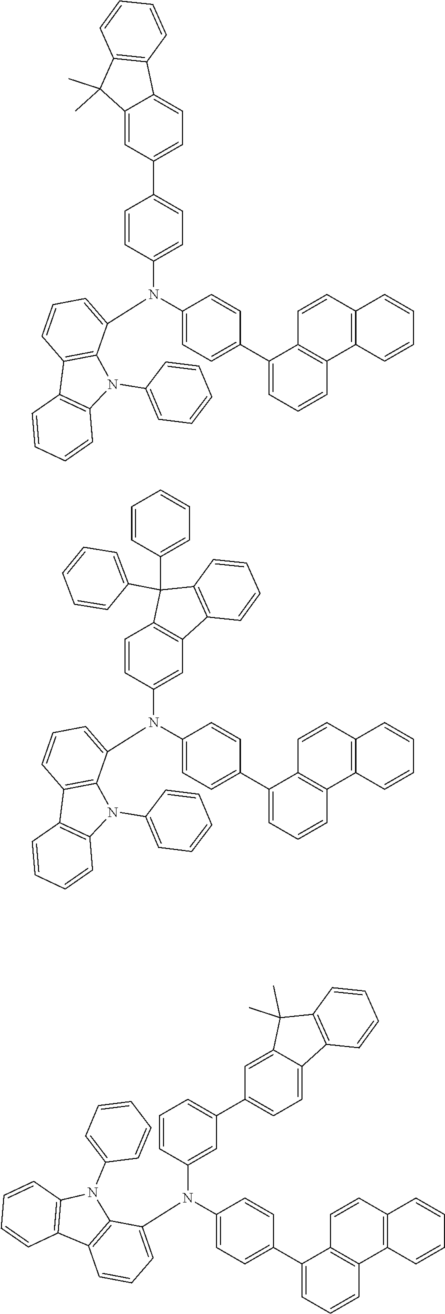 [SMILES: CC1(C)c2ccccc2-c2ccc(-c3ccc(N(c4ccc(-c5cccc6c5ccc5ccccc56)cc4)c4cccc5c6ccccc6n(-c6ccccc6)c45)cc3)cc21 ; c1ccc(-n2c3ccccc3c3cccc(N(c4ccc(-c5cccc6c5ccc5ccccc56)cc4)c4ccc5c(c4)-c4ccccc4C5(c4ccccc4)c4ccccc4)c32)cc1 ; CC1(C)c2ccccc2-c2ccc(-c3cccc(N(c4ccc(-c5cccc6c5ccc5ccccc56)cc4)c4cccc5c6ccccc6n(-c6ccccc6)c45)c3)cc21]

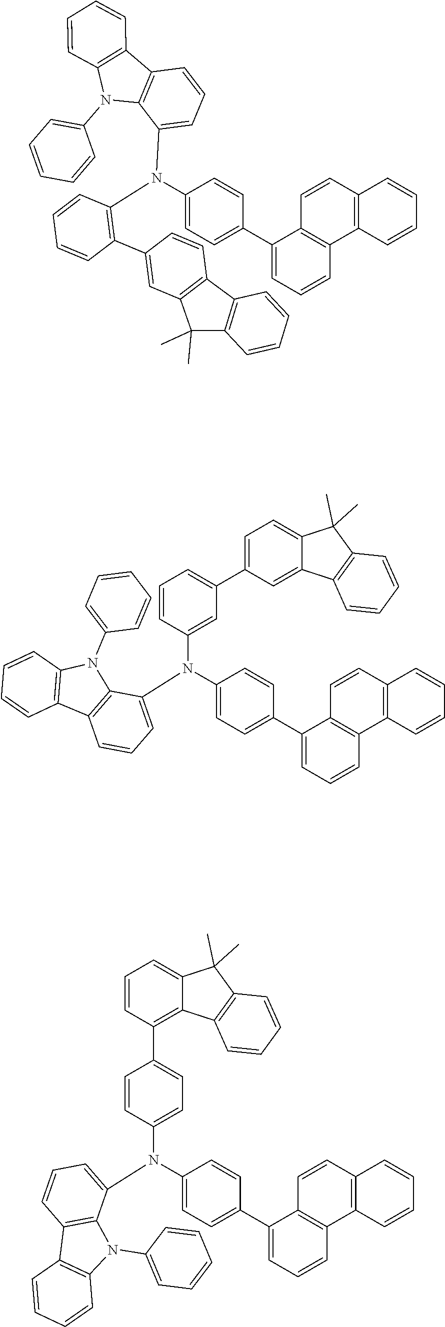 [SMILES: CC1(C)c2ccccc2-c2ccc(-c3ccccc3N(c3ccc(-c4cccc5c4ccc4ccccc45)cc3)c3cccc4c5ccccc5n(-c5ccccc5)c34)cc21 ; CC1(C)c2ccccc2-c2cc(-c3cccc(N(c4ccc(-c5cccc6c5ccc5ccccc56)cc4)c4cccc5c6ccccc6n(-c6ccccc6)c45)c3)ccc21 ; CC1(C)c2ccccc2-c2c(-c3ccc(N(c4ccc(-c5cccc6c5ccc5ccccc56)cc4)c4cccc5c6ccccc6n(-c6ccccc6)c45)cc3)cccc21]

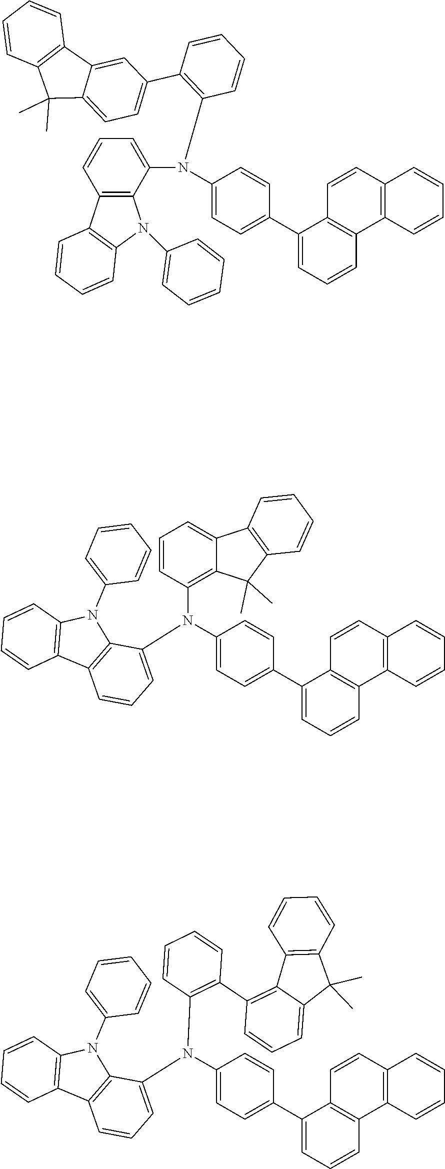 [SMILES: CC1(C)c2ccccc2-c2cc(-c3ccccc3N(c3ccc(-c4cccc5c4ccc4ccccc45)cc3)c3cccc4c5ccccc5n(-c5ccccc5)c34)ccc21 ; CC1(C)c2ccccc2-c2cccc(N(c3ccc(-c4cccc5c4ccc4ccccc45)cc3)c3cccc4c5ccccc5n(-c5ccccc5)c34)c21 ; CC1(C)c2ccccc2-c2c(-c3ccccc3N(c3ccc(-c4cccc5c4ccc4ccccc45)cc3)c3cccc4c5ccccc5n(-c5ccccc5)c34)cccc21]

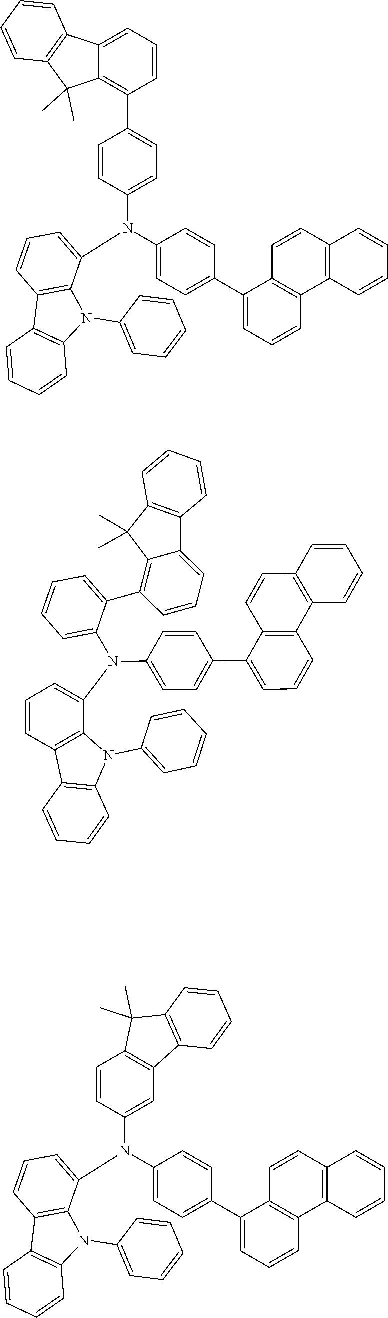 [SMILES: CC1(C)c2ccccc2-c2cccc(-c3ccc(N(c4ccc(-c5cccc6c5ccc5ccccc56)cc4)c4cccc5c6ccccc6n(-c6ccccc6)c45)cc3)c21 ; CC1(C)c2ccccc2-c2cccc(-c3ccccc3N(c3ccc(-c4cccc5c4ccc4ccccc45)cc3)c3cccc4c5ccccc5n(-c5ccccc5)c34)c21 ; CC1(C)c2ccccc2-c2cc(N(c3ccc(-c4cccc5c4ccc4ccccc45)cc3)c3cccc4c5ccccc5n(-c5ccccc5)c34)ccc21]

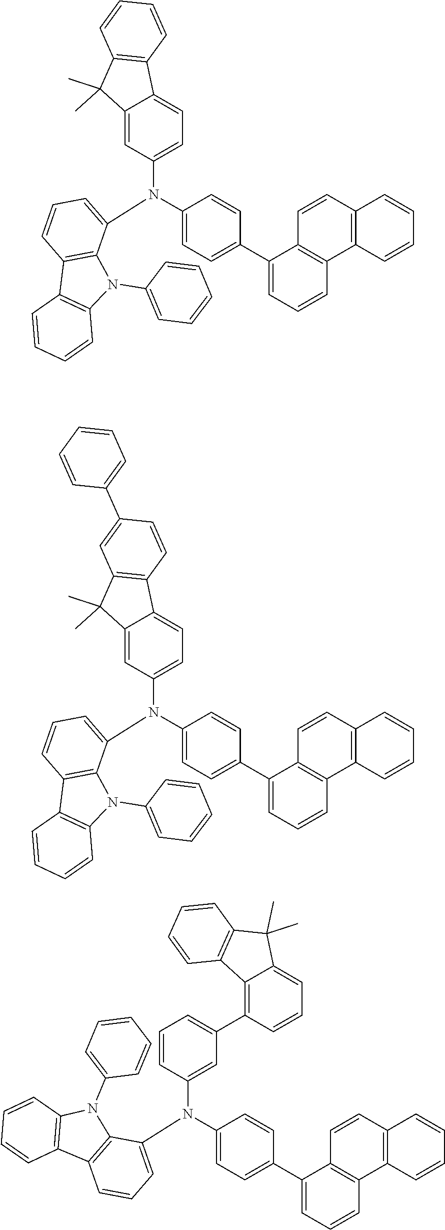 [SMILES: CC1(C)c2ccccc2-c2ccc(N(c3ccc(-c4cccc5c4ccc4ccccc45)cc3)c3cccc4c5ccccc5n(-c5ccccc5)c34)cc21 ; CC1(C)c2cc(-c3ccccc3)ccc2-c2ccc(N(c3ccc(-c4cccc5c4ccc4ccccc45)cc3)c3cccc4c5ccccc5n(-c5ccccc5)c34)cc21 ; CC1(C)c2ccccc2-c2c(-c3cccc(N(c4ccc(-c5cccc6c5ccc5ccccc56)cc4)c4cccc5c6ccccc6n(-c6ccccc6)c45)c3)cccc21]

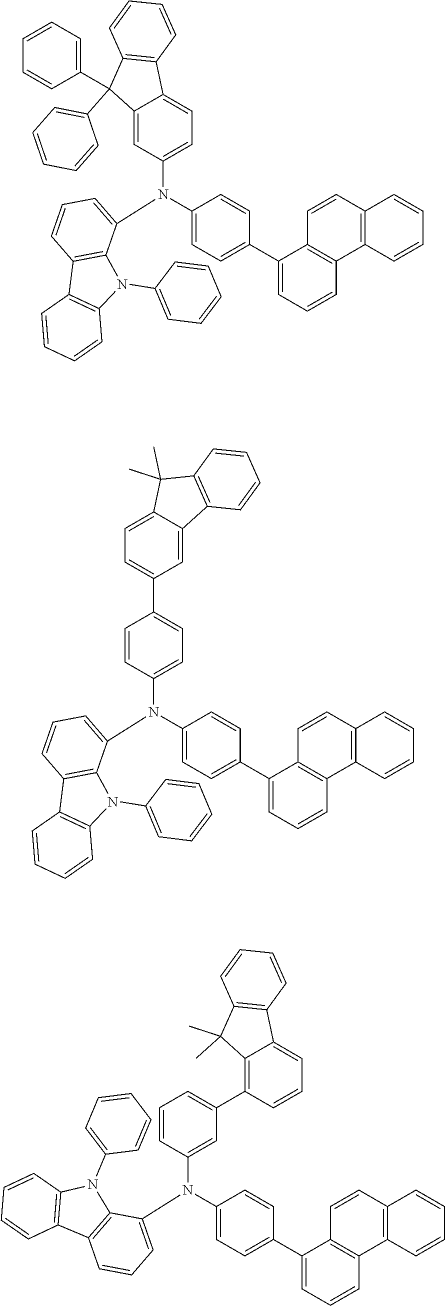 [SMILES: c1ccc(-n2c3ccccc3c3cccc(N(c4ccc(-c5cccc6c5ccc5ccccc56)cc4)c4ccc5c(c4)C(c4ccccc4)(c4ccccc4)c4ccccc4-5)c32)cc1 ; CC1(C)c2ccccc2-c2cc(-c3ccc(N(c4ccc(-c5cccc6c5ccc5ccccc56)cc4)c4cccc5c6ccccc6n(-c6ccccc6)c45)cc3)ccc21 ; CC1(C)c2ccccc2-c2cccc(-c3cccc(N(c4ccc(-c5cccc6c5ccc5ccccc56)cc4)c4cccc5c6ccccc6n(-c6ccccc6)c45)c3)c21]

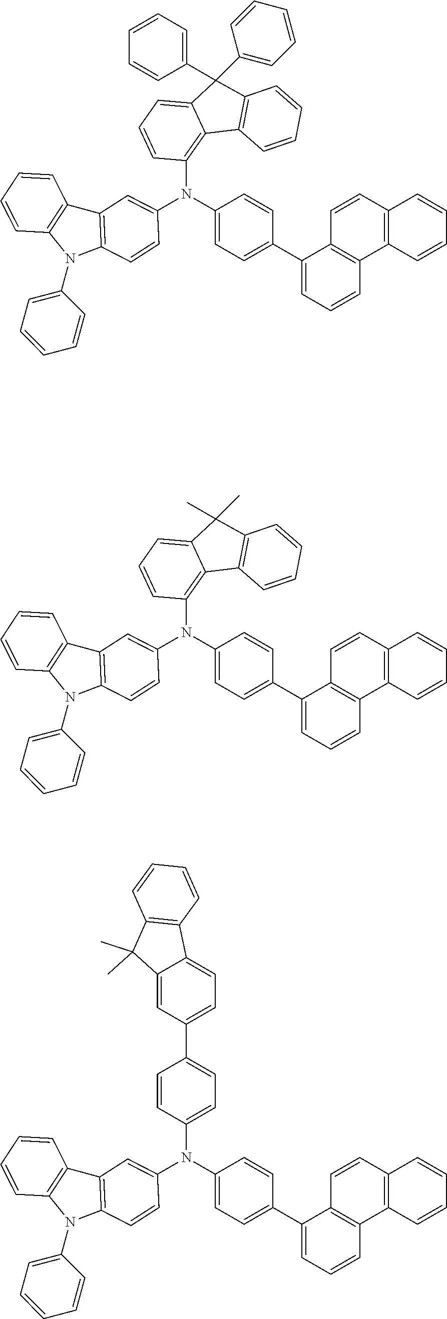 [SMILES: c1ccc(-n2c3ccccc3c3cc(N(c4ccc(-c5cccc6c5ccc5ccccc56)cc4)c4cccc5c4-c4ccccc4C5(c4ccccc4)c4ccccc4)ccc32)cc1 ; CC1(C)c2ccccc2-c2c(N(c3ccc(-c4cccc5c4ccc4ccccc45)cc3)c3ccc4c(c3)c3ccccc3n4-c3ccccc3)cccc21 ; CC1(C)c2ccccc2-c2ccc(-c3ccc(N(c4ccc(-c5cccc6c5ccc5ccccc56)cc4)c4ccc5c(c4)c4ccccc4n5-c4ccccc4)cc3)cc21]

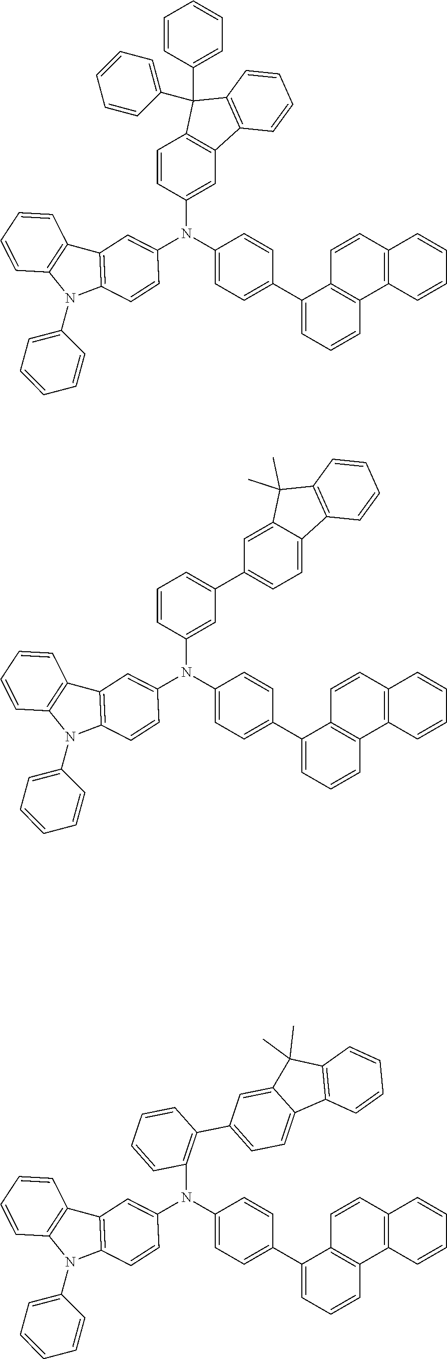 [SMILES: c1ccc(-n2c3ccccc3c3cc(N(c4ccc(-c5cccc6c5ccc5ccccc56)cc4)c4ccc5c(c4)-c4ccccc4C5(c4ccccc4)c4ccccc4)ccc32)cc1 ; CC1(C)c2ccccc2-c2ccc(-c3cccc(N(c4ccc(-c5cccc6c5ccc5ccccc56)cc4)c4ccc5c(c4)c4ccccc4n5-c4ccccc4)c3)cc21 ; CC1(C)c2ccccc2-c2ccc(-c3ccccc3N(c3ccc(-c4cccc5c4ccc4ccccc45)cc3)c3ccc4c(c3)c3ccccc3n4-c3ccccc3)cc21]

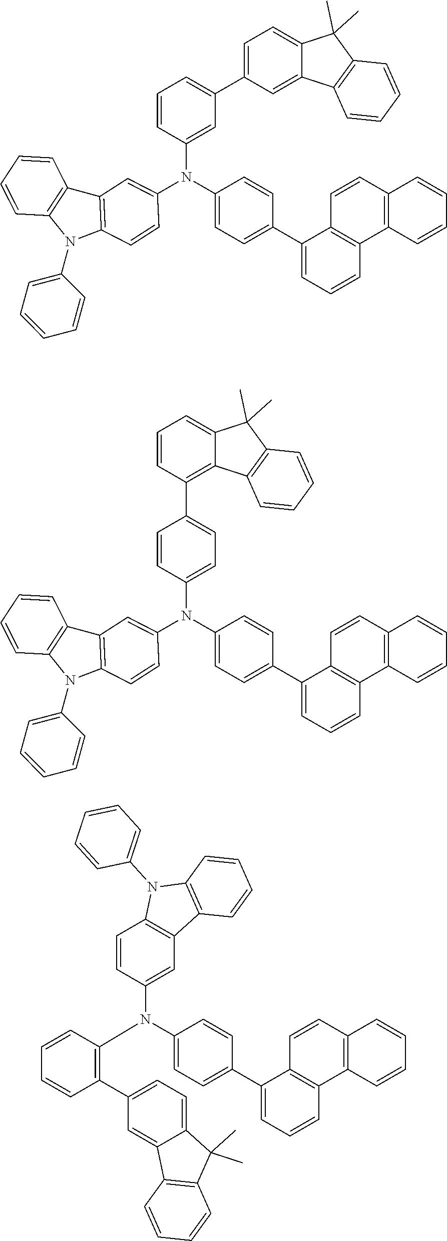

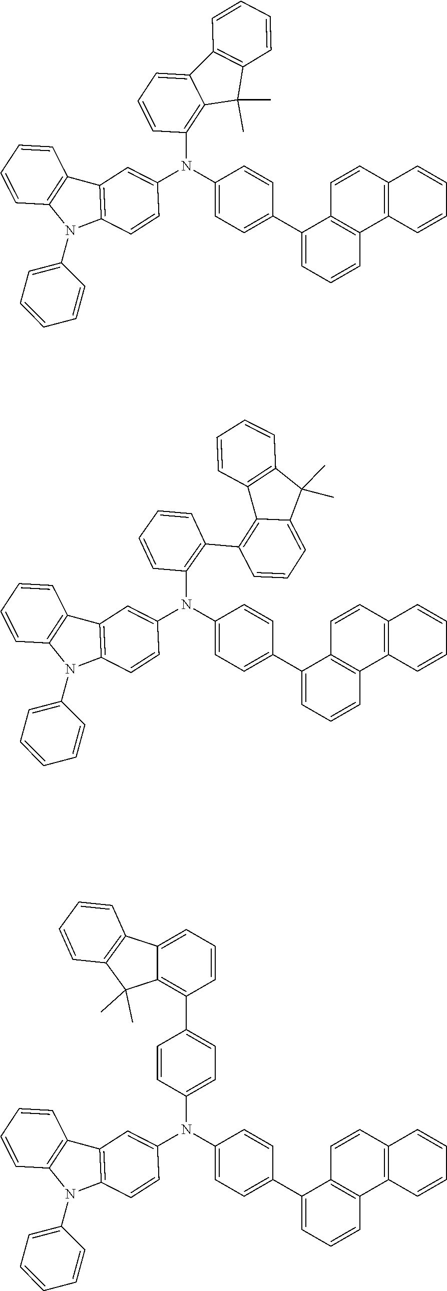 [SMILES: CC1(C)c2ccccc2-c2cccc(N(c3ccc(-c4cccc5c4ccc4ccccc45)cc3)c3ccc4c(c3)c3ccccc3n4-c3ccccc3)c21 ; CC1(C)c2ccccc2-c2c(-c3ccccc3N(c3ccc(-c4cccc5c4ccc4ccccc45)cc3)c3ccc4c(c3)c3ccccc3n4-c3ccccc3)cccc21 ; CC1(C)c2ccccc2-c2cccc(-c3ccc(N(c4ccc(-c5cccc6c5ccc5ccccc56)cc4)c4ccc5c(c4)c4ccccc4n5-c4ccccc4)cc3)c21]

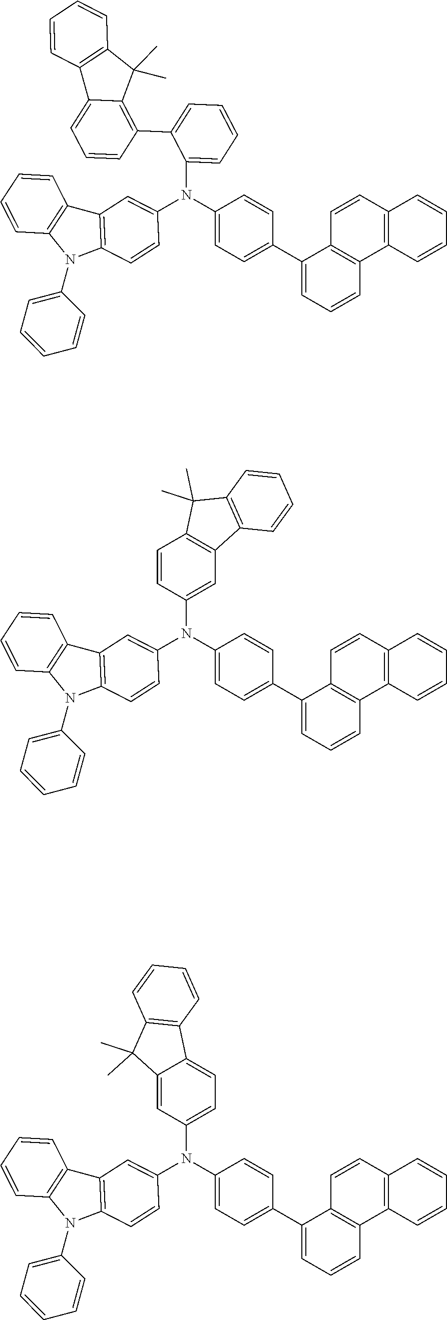 [SMILES: CC1(C)c2ccccc2-c2cccc(-c3ccccc3N(c3ccc(-c4cccc5c4ccc4ccccc45)cc3)c3ccc4c(c3)c3ccccc3n4-c3ccccc3)c21 ; CC1(C)c2ccccc2-c2cc(N(c3ccc(-c4cccc5c4ccc4ccccc45)cc3)c3ccc4c(c3)c3ccccc3n4-c3ccccc3)ccc21 ; CC1(C)c2ccccc2-c2ccc(N(c3ccc(-c4cccc5c4ccc4ccccc45)cc3)c3ccc4c(c3)c3ccccc3n4-c3ccccc3)cc21]

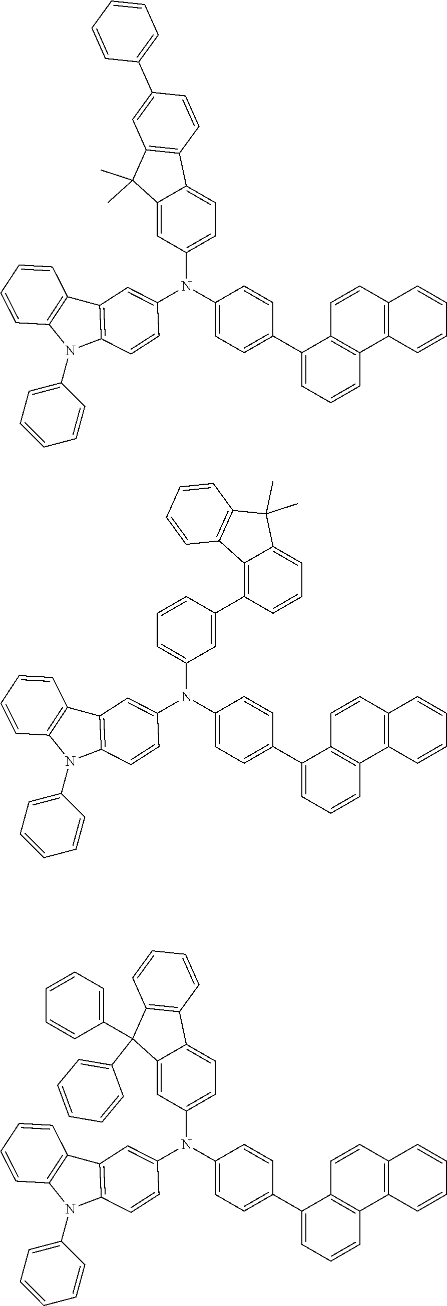 [SMILES: CC1(C)c2cc(-c3ccccc3)ccc2-c2ccc(N(c3ccc(-c4cccc5c4ccc4ccccc45)cc3)c3ccc4c(c3)c3ccccc3n4-c3ccccc3)cc21 ; CC1(C)c2ccccc2-c2c(-c3cccc(N(c4ccc(-c5cccc6c5ccc5ccccc56)cc4)c4ccc5c(c4)c4ccccc4n5-c4ccccc4)c3)cccc21 ; c1ccc(-n2c3ccccc3c3cc(N(c4ccc(-c5cccc6c5ccc5ccccc56)cc4)c4ccc5c(c4)C(c4ccccc4)(c4ccccc4)c4ccccc4-5)ccc32)cc1]

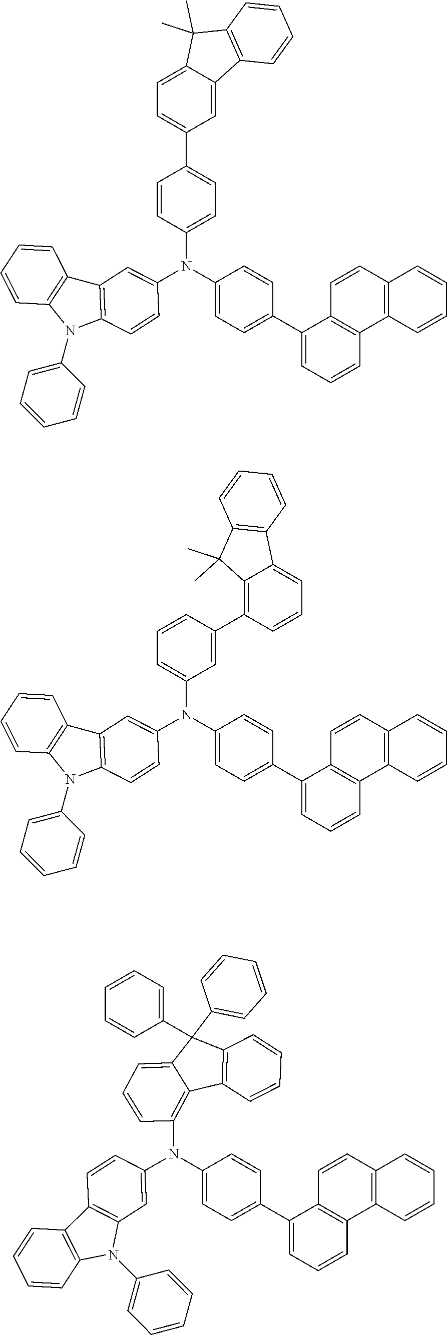 [SMILES: CC1(C)c2ccccc2-c2cc(-c3ccc(N(c4ccc(-c5cccc6c5ccc5ccccc56)cc4)c4ccc5c(c4)c4ccccc4n5-c4ccccc4)cc3)ccc21 ; CC1(C)c2ccccc2-c2cccc(-c3cccc(N(c4ccc(-c5cccc6c5ccc5ccccc56)cc4)c4ccc5c(c4)c4ccccc4n5-c4ccccc4)c3)c21 ; c1ccc(-n2c3ccccc3c3ccc(N(c4ccc(-c5cccc6c5ccc5ccccc56)cc4)c4cccc5c4-c4ccccc4C5(c4ccccc4)c4ccccc4)cc32)cc1]

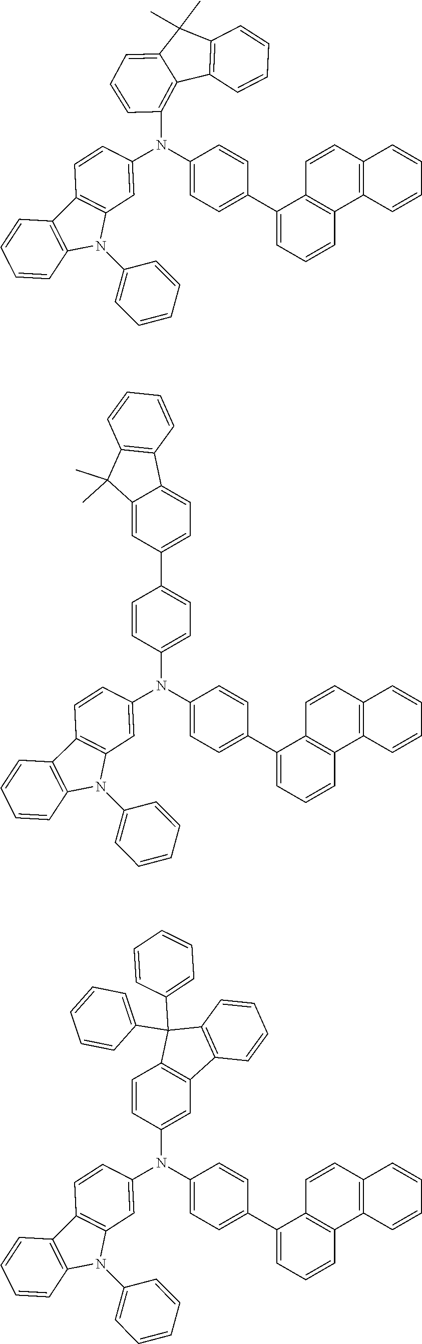 [SMILES: CC1(C)c2ccccc2-c2c(N(c3ccc(-c4cccc5c4ccc4ccccc45)cc3)c3ccc4c5ccccc5n(-c5ccccc5)c4c3)cccc21 ; CC1(C)c2ccccc2-c2ccc(-c3ccc(N(c4ccc(-c5cccc6c5ccc5ccccc56)cc4)c4ccc5c6ccccc6n(-c6ccccc6)c5c4)cc3)cc21 ; c1ccc(-n2c3ccccc3c3ccc(N(c4ccc(-c5cccc6c5ccc5ccccc56)cc4)c4ccc5c(c4)-c4ccccc4C5(c4ccccc4)c4ccccc4)cc32)cc1]

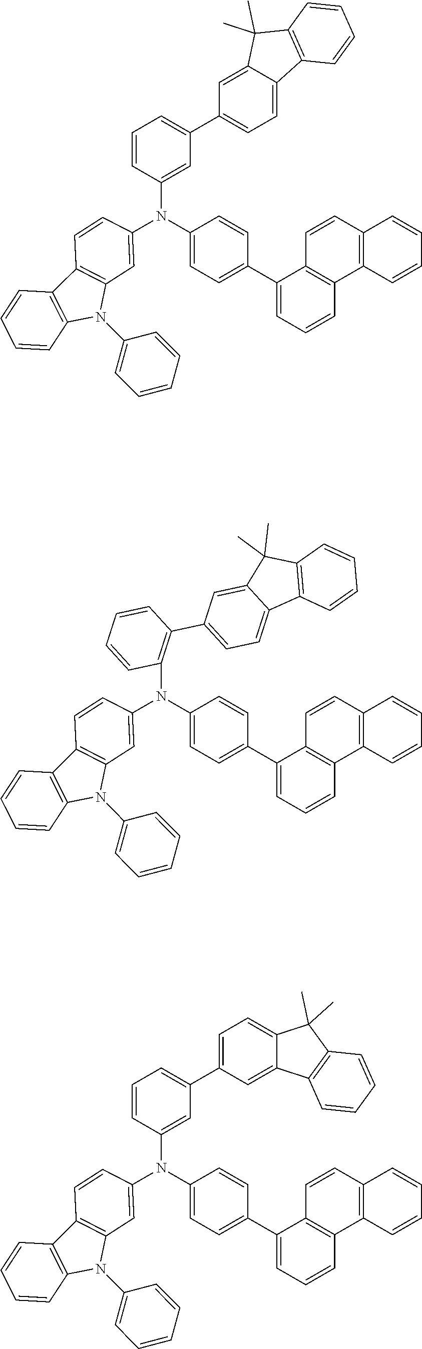 [SMILES: CC1(C)c2ccccc2-c2ccc(-c3cccc(N(c4ccc(-c5cccc6c5ccc5ccccc56)cc4)c4ccc5c6ccccc6n(-c6ccccc6)c5c4)c3)cc21 ; CC1(C)c2ccccc2-c2ccc(-c3ccccc3N(c3ccc(-c4cccc5c4ccc4ccccc45)cc3)c3ccc4c5ccccc5n(-c5ccccc5)c4c3)cc21 ; CC1(C)c2ccccc2-c2cc(-c3cccc(N(c4ccc(-c5cccc6c5ccc5ccccc56)cc4)c4ccc5c6ccccc6n(-c6ccccc6)c5c4)c3)ccc21]

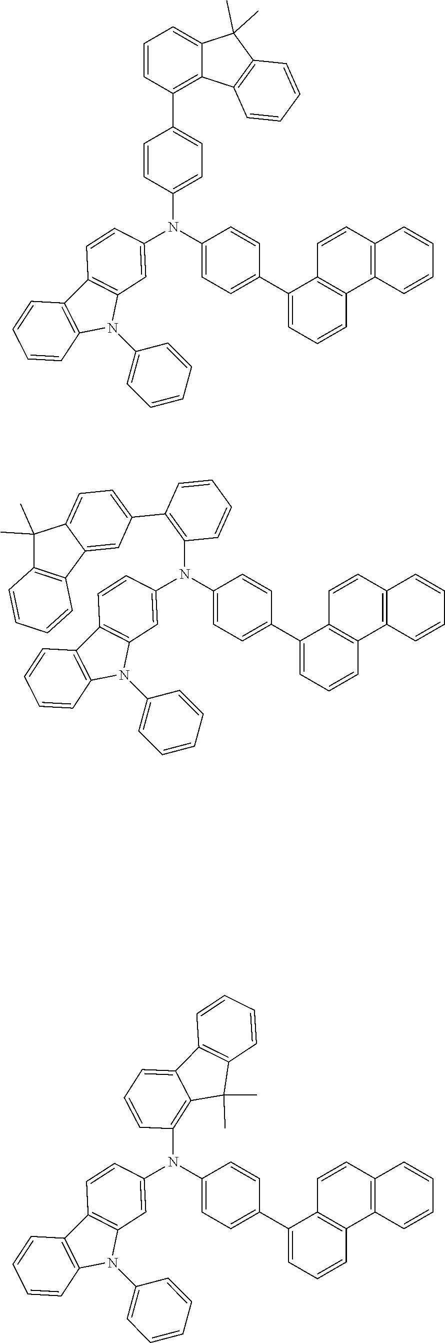 [SMILES: CC1(C)c2ccccc2-c2c(-c3ccc(N(c4ccc(-c5cccc6c5ccc5ccccc56)cc4)c4ccc5c6ccccc6n(-c6ccccc6)c5c4)cc3)cccc21 ; CC1(C)c2ccccc2-c2cc(-c3ccccc3N(c3ccc(-c4cccc5c4ccc4ccccc45)cc3)c3ccc4c5ccccc5n(-c5ccccc5)c4c3)ccc21 ; CC1(C)c2ccccc2-c2cccc(N(c3ccc(-c4cccc5c4ccc4ccccc45)cc3)c3ccc4c5ccccc5n(-c5ccccc5)c4c3)c21]

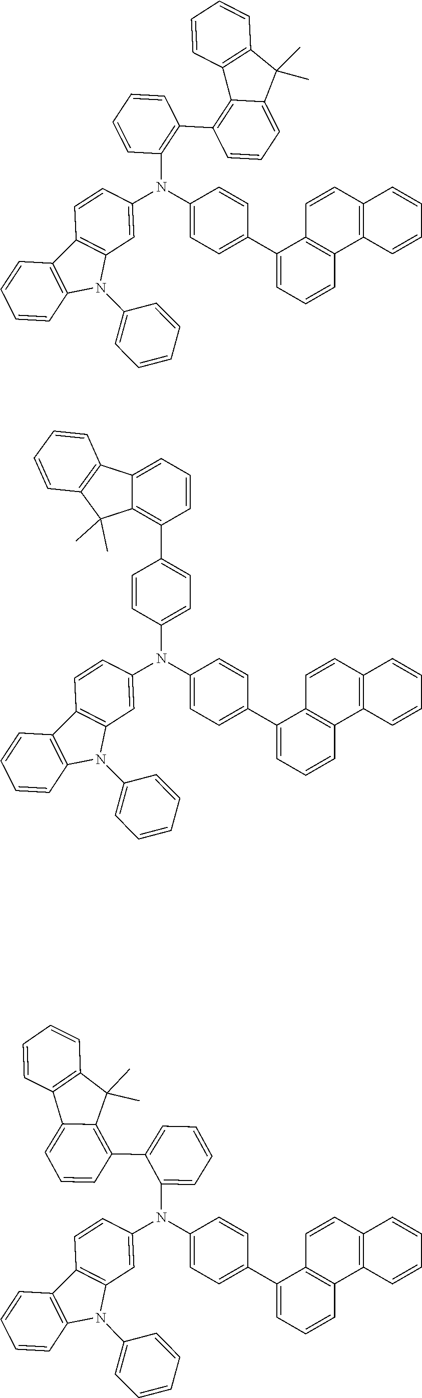 [SMILES: CC1(C)c2ccccc2-c2c(-c3ccccc3N(c3ccc(-c4cccc5c4ccc4ccccc45)cc3)c3ccc4c5ccccc5n(-c5ccccc5)c4c3)cccc21 ; CC1(C)c2ccccc2-c2cccc(-c3ccc(N(c4ccc(-c5cccc6c5ccc5ccccc56)cc4)c4ccc5c6ccccc6n(-c6ccccc6)c5c4)cc3)c21 ; CC1(C)c2ccccc2-c2cccc(-c3ccccc3N(c3ccc(-c4cccc5c4ccc4ccccc45)cc3)c3ccc4c5ccccc5n(-c5ccccc5)c4c3)c21]

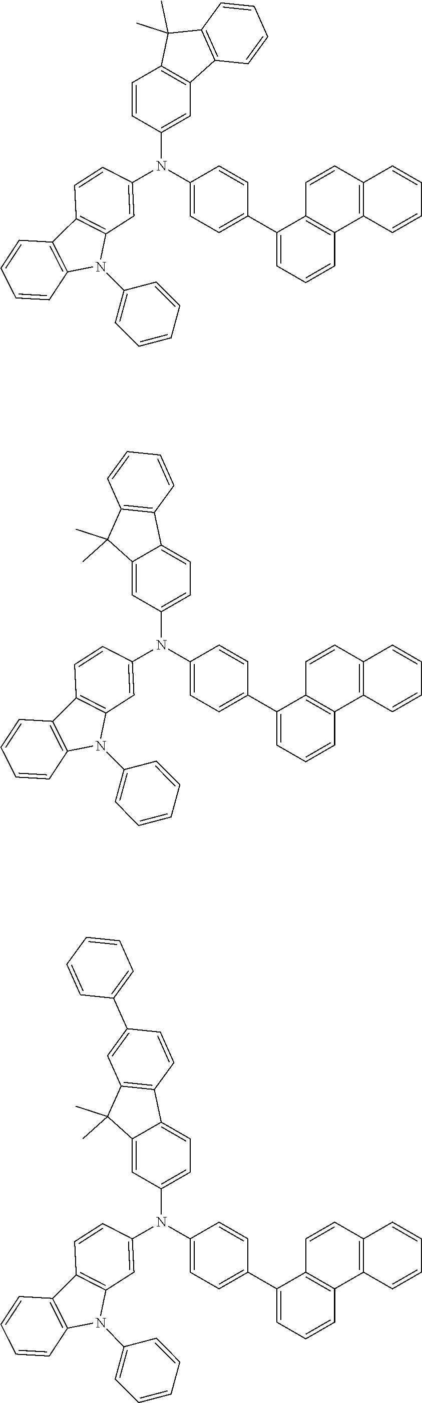 [SMILES: CC1(C)c2ccccc2-c2cc(N(c3ccc(-c4cccc5c4ccc4ccccc45)cc3)c3ccc4c5ccccc5n(-c5ccccc5)c4c3)ccc21 ; CC1(C)c2ccccc2-c2ccc(N(c3ccc(-c4cccc5c4ccc4ccccc45)cc3)c3ccc4c5ccccc5n(-c5ccccc5)c4c3)cc21 ; CC1(C)c2cc(-c3ccccc3)ccc2-c2ccc(N(c3ccc(-c4cccc5c4ccc4ccccc45)cc3)c3ccc4c5ccccc5n(-c5ccccc5)c4c3)cc21]

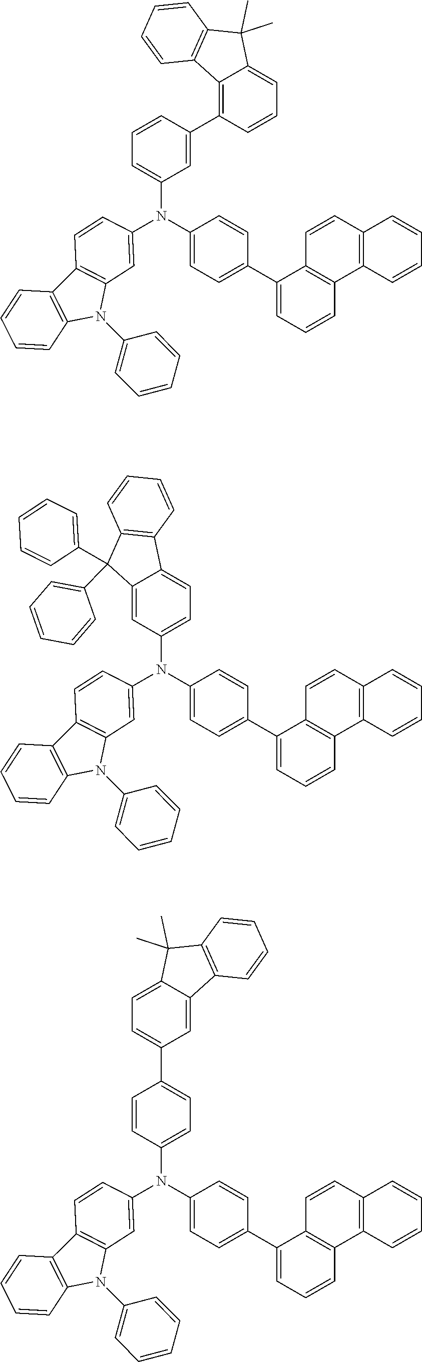 [SMILES: CC1(C)c2ccccc2-c2c(-c3cccc(N(c4ccc(-c5cccc6c5ccc5ccccc56)cc4)c4ccc5c6ccccc6n(-c6ccccc6)c5c4)c3)cccc21 ; c1ccc(-n2c3ccccc3c3ccc(N(c4ccc(-c5cccc6c5ccc5ccccc56)cc4)c4ccc5c(c4)C(c4ccccc4)(c4ccccc4)c4ccccc4-5)cc32)cc1 ; CC1(C)c2ccccc2-c2cc(-c3ccc(N(c4ccc(-c5cccc6c5ccc5ccccc56)cc4)c4ccc5c6ccccc6n(-c6ccccc6)c5c4)cc3)ccc21]

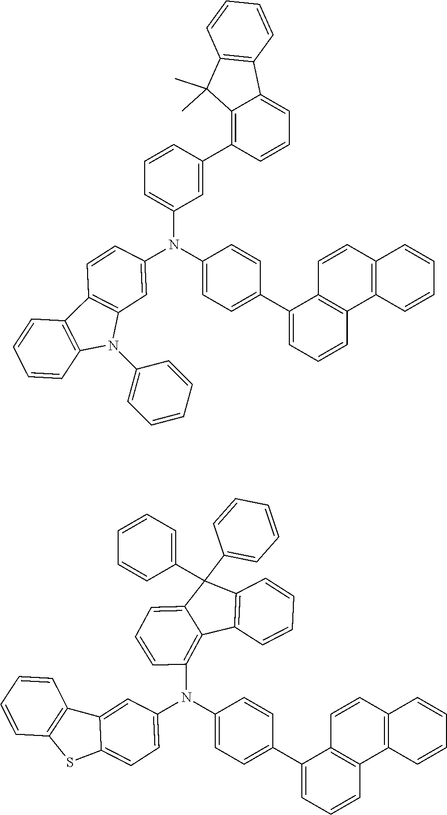 [SMILES: CC1(C)c2ccccc2-c2cccc(-c3cccc(N(c4ccc(-c5cccc6c5ccc5ccccc56)cc4)c4ccc5c6ccccc6n(-c6ccccc6)c5c4)c3)c21 ; c1ccc(C2(c3ccccc3)c3ccccc3-c3c(N(c4ccc(-c5cccc6c5ccc5ccccc56)cc4)c4ccc5sc6ccccc6c5c4)cccc32)cc1]

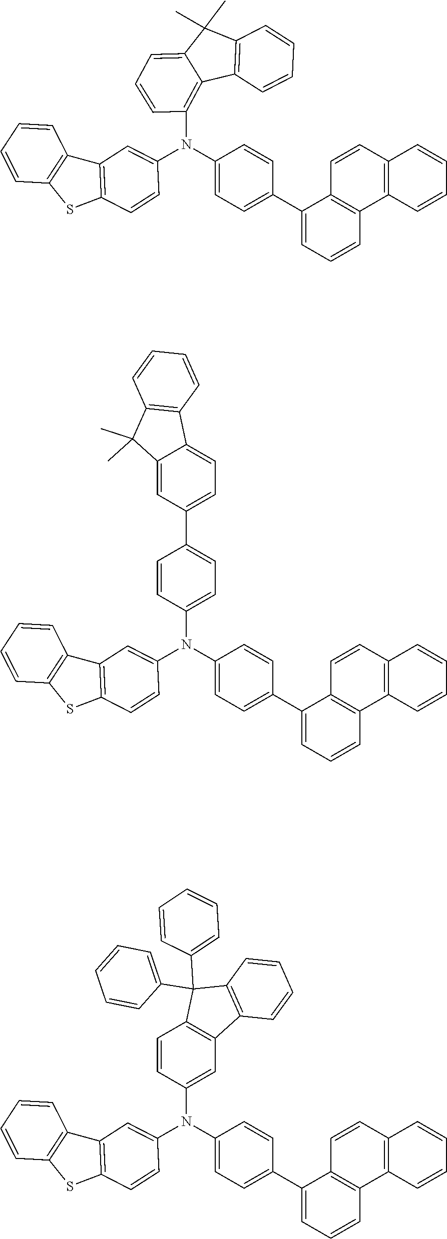 [SMILES: CC1(C)c2ccccc2-c2c(N(c3ccc(-c4cccc5c4ccc4ccccc45)cc3)c3ccc4sc5ccccc5c4c3)cccc21 ; CC1(C)c2ccccc2-c2ccc(-c3ccc(N(c4ccc(-c5cccc6c5ccc5ccccc56)cc4)c4ccc5sc6ccccc6c5c4)cc3)cc21 ; c1ccc(C2(c3ccccc3)c3ccccc3-c3cc(N(c4ccc(-c5cccc6c5ccc5ccccc56)cc4)c4ccc5sc6ccccc6c5c4)ccc32)cc1]

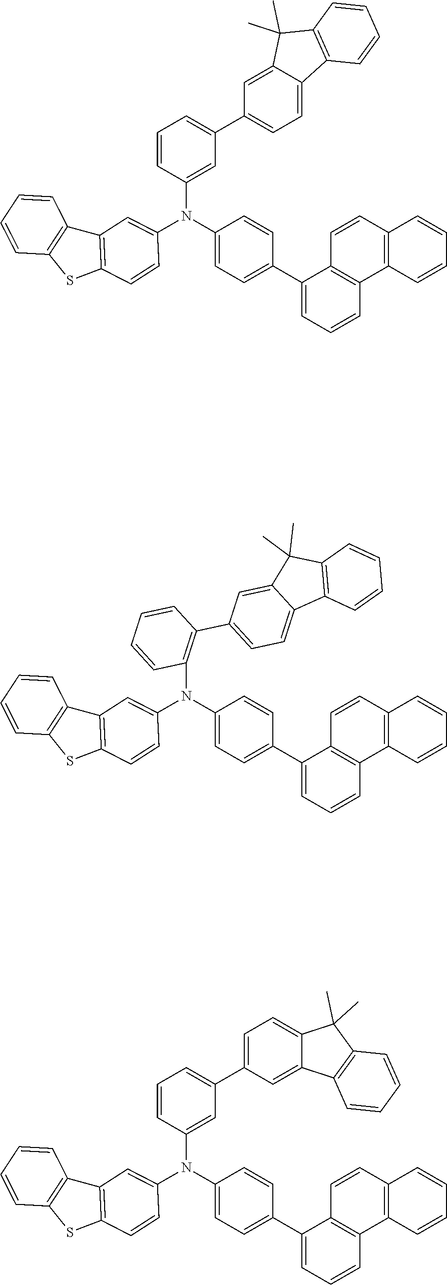 [SMILES: CC1(C)c2ccccc2-c2ccc(-c3cccc(N(c4ccc(-c5cccc6c5ccc5ccccc56)cc4)c4ccc5sc6ccccc6c5c4)c3)cc21 ; CC1(C)c2ccccc2-c2ccc(-c3ccccc3N(c3ccc(-c4cccc5c4ccc4ccccc45)cc3)c3ccc4sc5ccccc5c4c3)cc21 ; CC1(C)c2ccccc2-c2cc(-c3cccc(N(c4ccc(-c5cccc6c5ccc5ccccc56)cc4)c4ccc5sc6ccccc6c5c4)c3)ccc21]

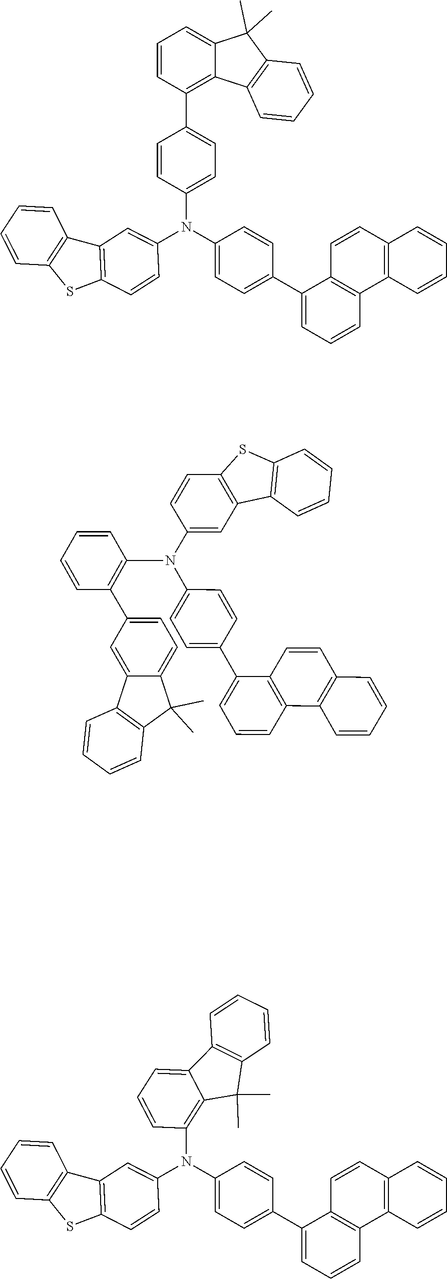 [SMILES: CC1(C)c2ccccc2-c2c(-c3ccc(N(c4ccc(-c5cccc6c5ccc5ccccc56)cc4)c4ccc5sc6ccccc6c5c4)cc3)cccc21 ; CC1(C)c2ccccc2-c2cc(-c3ccccc3N(c3ccc(-c4cccc5c4ccc4ccccc45)cc3)c3ccc4sc5ccccc5c4c3)ccc21 ; CC1(C)c2ccccc2-c2cccc(N(c3ccc(-c4cccc5c4ccc4ccccc45)cc3)c3ccc4sc5ccccc5c4c3)c21]

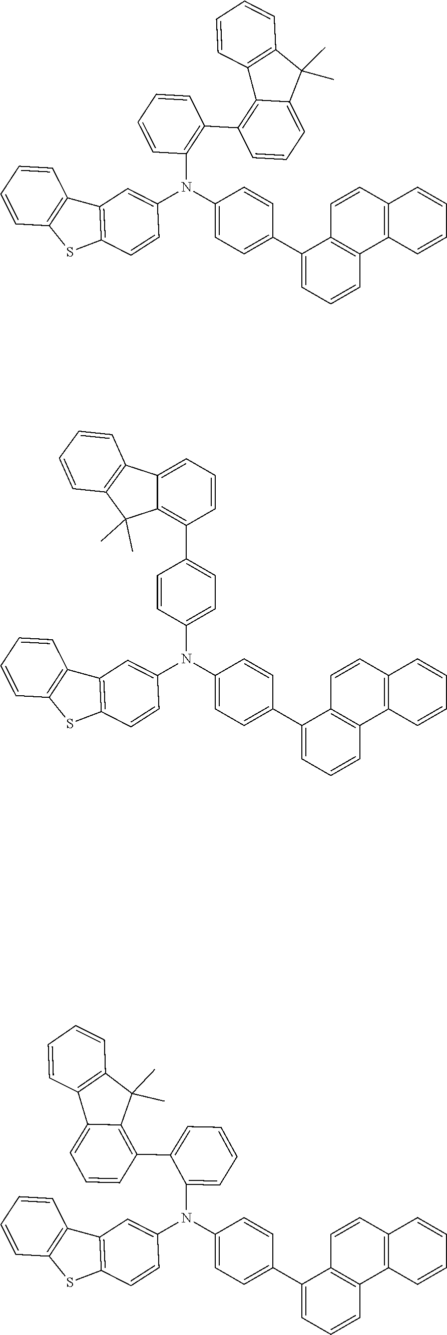 [SMILES: CC1(C)c2ccccc2-c2c(-c3ccccc3N(c3ccc(-c4cccc5c4ccc4ccccc45)cc3)c3ccc4sc5ccccc5c4c3)cccc21 ; CC1(C)c2ccccc2-c2cccc(-c3ccc(N(c4ccc(-c5cccc6c5ccc5ccccc56)cc4)c4ccc5sc6ccccc6c5c4)cc3)c21 ; CC1(C)c2ccccc2-c2cccc(-c3ccccc3N(c3ccc(-c4cccc5c4ccc4ccccc45)cc3)c3ccc4sc5ccccc5c4c3)c21]

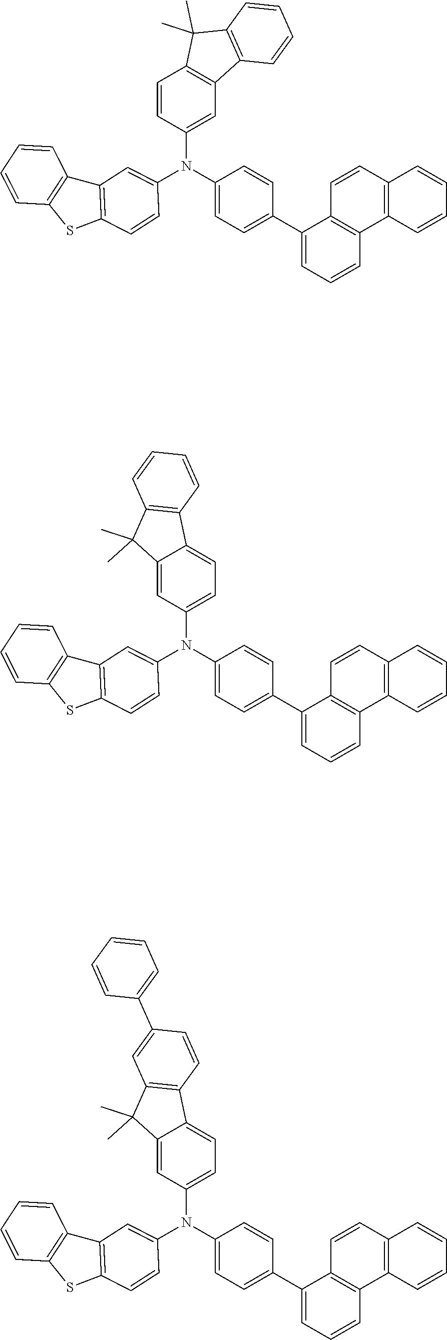 [SMILES: CC1(C)c2ccccc2-c2cc(N(c3ccc(-c4cccc5c4ccc4ccccc45)cc3)c3ccc4sc5ccccc5c4c3)ccc21 ; CC1(C)c2ccccc2-c2ccc(N(c3ccc(-c4cccc5c4ccc4ccccc45)cc3)c3ccc4sc5ccccc5c4c3)cc21 ; CC1(C)c2cc(-c3ccccc3)ccc2-c2ccc(N(c3ccc(-c4cccc5c4ccc4ccccc45)cc3)c3ccc4sc5ccccc5c4c3)cc21]

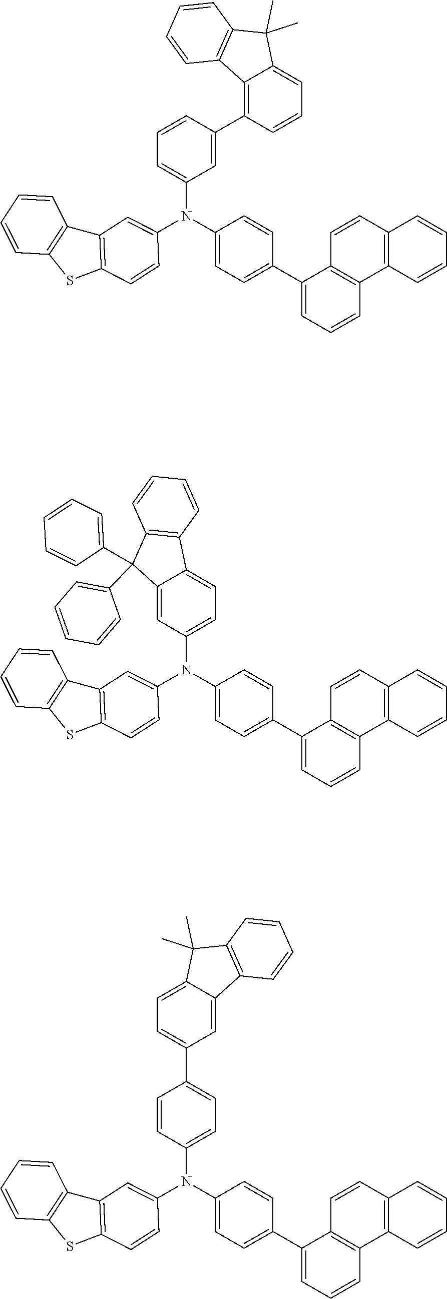 [SMILES: CC1(C)c2ccccc2-c2c(-c3cccc(N(c4ccc(-c5cccc6c5ccc5ccccc56)cc4)c4ccc5sc6ccccc6c5c4)c3)cccc21 ; c1ccc(C2(c3ccccc3)c3ccccc3-c3ccc(N(c4ccc(-c5cccc6c5ccc5ccccc56)cc4)c4ccc5sc6ccccc6c5c4)cc32)cc1 ; CC1(C)c2ccccc2-c2cc(-c3ccc(N(c4ccc(-c5cccc6c5ccc5ccccc56)cc4)c4ccc5sc6ccccc6c5c4)cc3)ccc21]

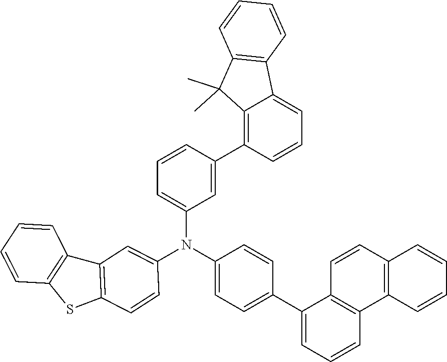 [SMILES: CC1(C)c2ccccc2-c2cccc(-c3cccc(N(c4ccc(-c5cccc6c5ccc5ccccc56)cc4)c4ccc5sc6ccccc6c5c4)c3)c21]

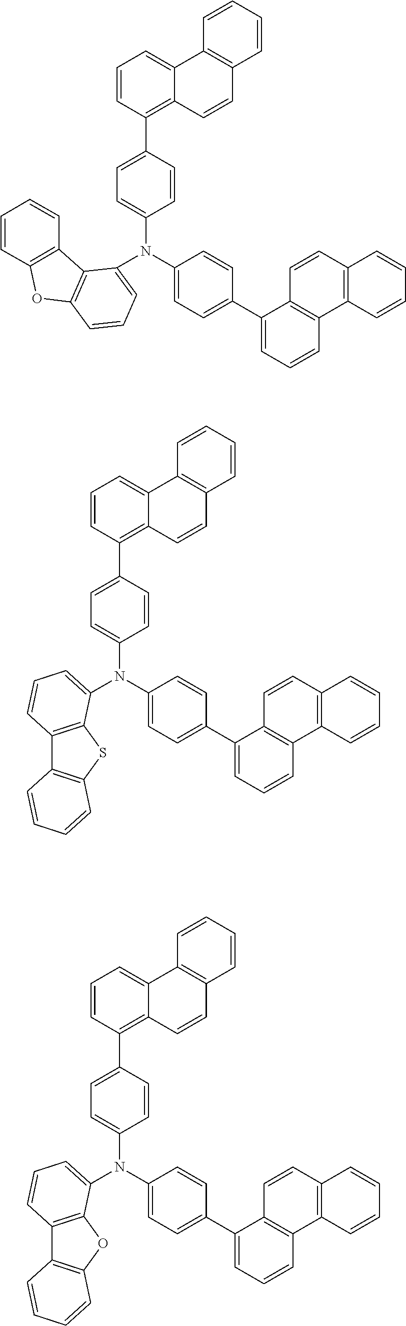 [SMILES: c1ccc2c(c1)ccc1c(-c3ccc(N(c4ccc(-c5cccc6c5ccc5ccccc56)cc4)c4cccc5oc6ccccc6c45)cc3)cccc12 ; c1ccc2c(c1)ccc1c(-c3ccc(N(c4ccc(-c5cccc6c5ccc5ccccc56)cc4)c4cccc5c4sc4ccccc45)cc3)cccc12 ; c1ccc2c(c1)ccc1c(-c3ccc(N(c4ccc(-c5cccc6c5ccc5ccccc56)cc4)c4cccc5c4oc4ccccc45)cc3)cccc12]

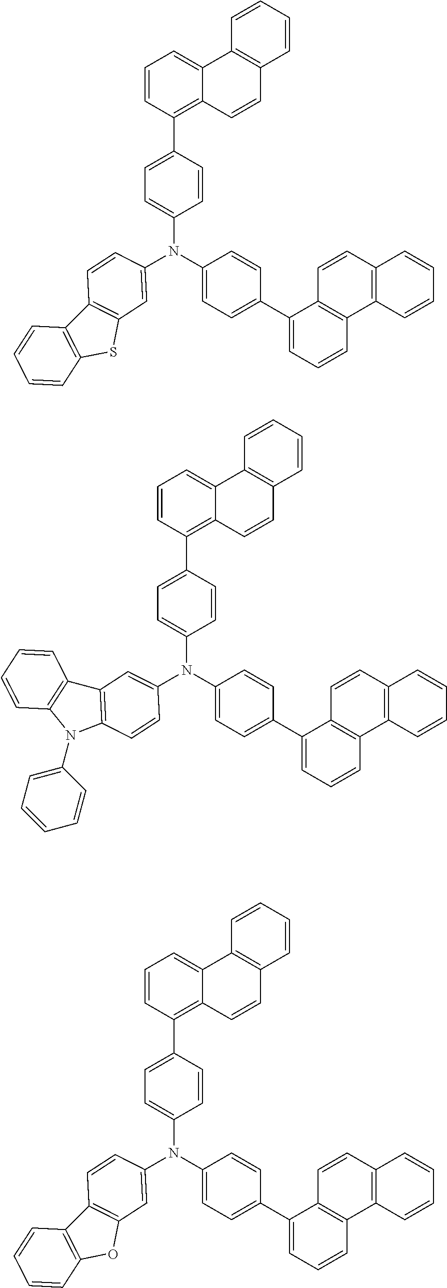 [SMILES: c1ccc2c(c1)ccc1c(-c3ccc(N(c4ccc(-c5cccc6c5ccc5ccccc56)cc4)c4ccc5c(c4)sc4ccccc45)cc3)cccc12 ; c1ccc(-n2c3ccccc3c3cc(N(c4ccc(-c5cccc6c5ccc5ccccc56)cc4)c4ccc(-c5cccc6c5ccc5ccccc56)cc4)ccc32)cc1 ; c1ccc2c(c1)ccc1c(-c3ccc(N(c4ccc(-c5cccc6c5ccc5ccccc56)cc4)c4ccc5c(c4)oc4ccccc45)cc3)cccc12]

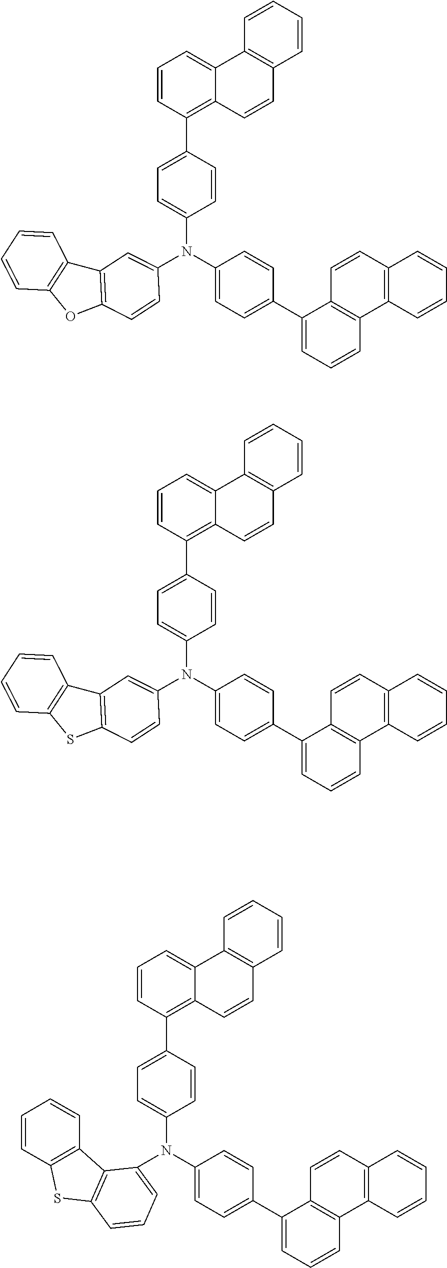 [SMILES: c1ccc2c(c1)ccc1c(-c3ccc(N(c4ccc(-c5cccc6c5ccc5ccccc56)cc4)c4ccc5oc6ccccc6c5c4)cc3)cccc12 ; c1ccc2c(c1)ccc1c(-c3ccc(N(c4ccc(-c5cccc6c5ccc5ccccc56)cc4)c4ccc5sc6ccccc6c5c4)cc3)cccc12 ; c1ccc2c(c1)ccc1c(-c3ccc(N(c4ccc(-c5cccc6c5ccc5ccccc56)cc4)c4cccc5sc6ccccc6c45)cc3)cccc12]

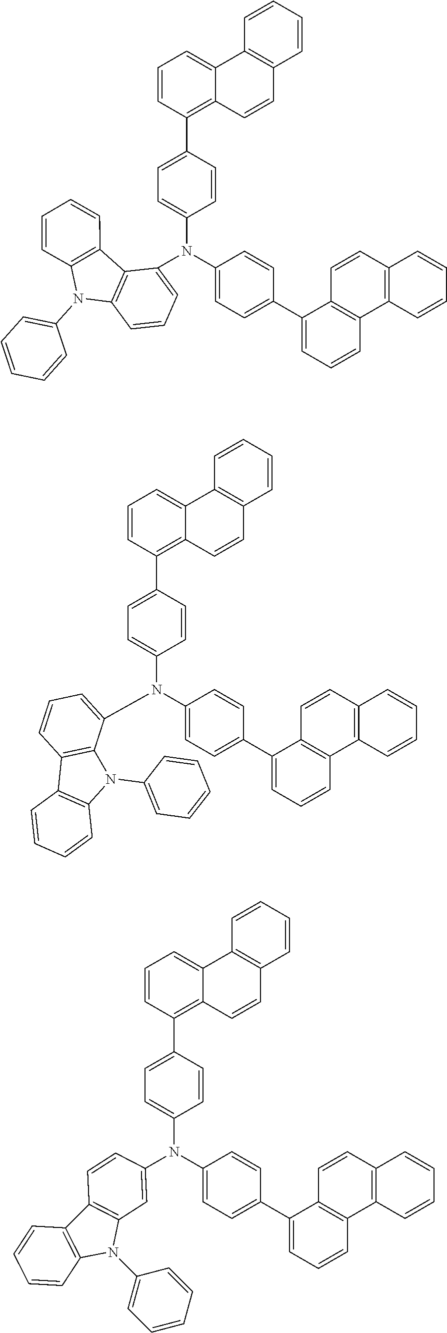 [SMILES: c1ccc(-n2c3ccccc3c3c(N(c4ccc(-c5cccc6c5ccc5ccccc56)cc4)c4ccc(-c5cccc6c5ccc5ccccc56)cc4)cccc32)cc1 ; c1ccc(-n2c3ccccc3c3cccc(N(c4ccc(-c5cccc6c5ccc5ccccc56)cc4)c4ccc(-c5cccc6c5ccc5ccccc56)cc4)c32)cc1 ; c1ccc(-n2c3ccccc3c3ccc(N(c4ccc(-c5cccc6c5ccc5ccccc56)cc4)c4ccc(-c5cccc6c5ccc5ccccc56)cc4)cc32)cc1]

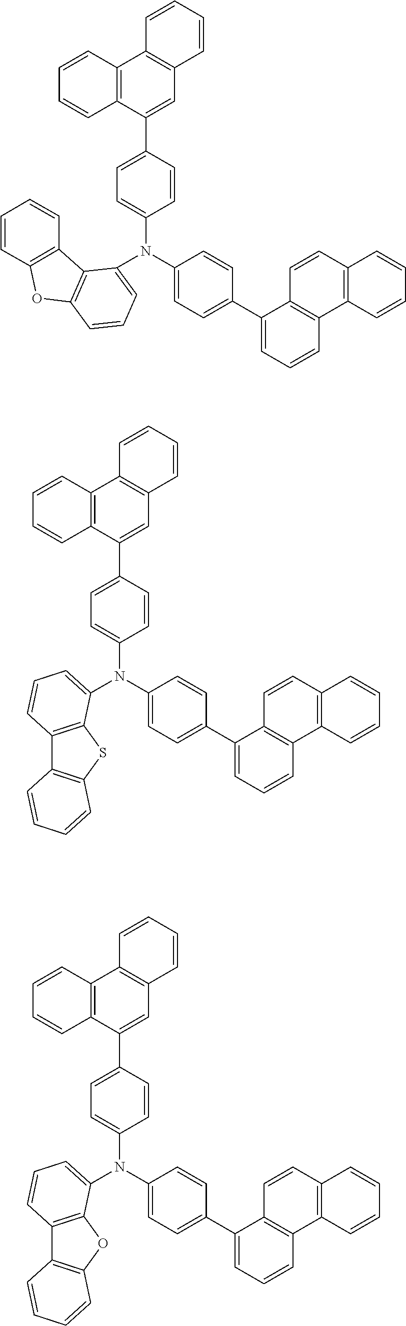 [SMILES: c1ccc2c(c1)ccc1c(-c3ccc(N(c4ccc(-c5cc6ccccc6c6ccccc56)cc4)c4cccc5oc6ccccc6c45)cc3)cccc12 ; c1ccc2c(c1)ccc1c(-c3ccc(N(c4ccc(-c5cc6ccccc6c6ccccc56)cc4)c4cccc5c4sc4ccccc45)cc3)cccc12 ; c1ccc2c(c1)ccc1c(-c3ccc(N(c4ccc(-c5cc6ccccc6c6ccccc56)cc4)c4cccc5c4oc4ccccc45)cc3)cccc12]

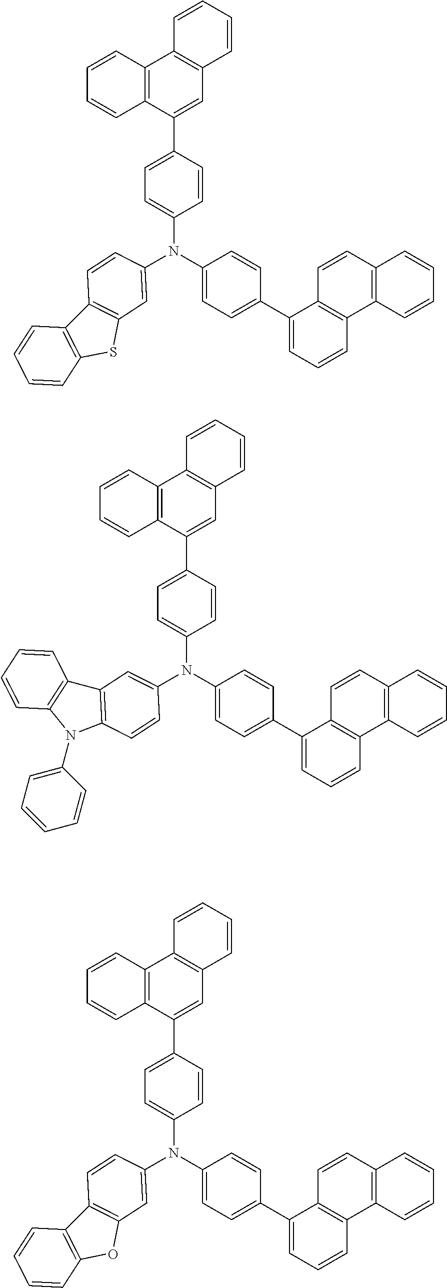 [SMILES: c1ccc2c(c1)ccc1c(-c3ccc(N(c4ccc(-c5cc6ccccc6c6ccccc56)cc4)c4ccc5c(c4)sc4ccccc45)cc3)cccc12 ; c1ccc(-n2c3ccccc3c3cc(N(c4ccc(-c5cccc6c5ccc5ccccc56)cc4)c4ccc(-c5cc6ccccc6c6ccccc56)cc4)ccc32)cc1 ; c1ccc2c(c1)ccc1c(-c3ccc(N(c4ccc(-c5cc6ccccc6c6ccccc56)cc4)c4ccc5c(c4)oc4ccccc45)cc3)cccc12]

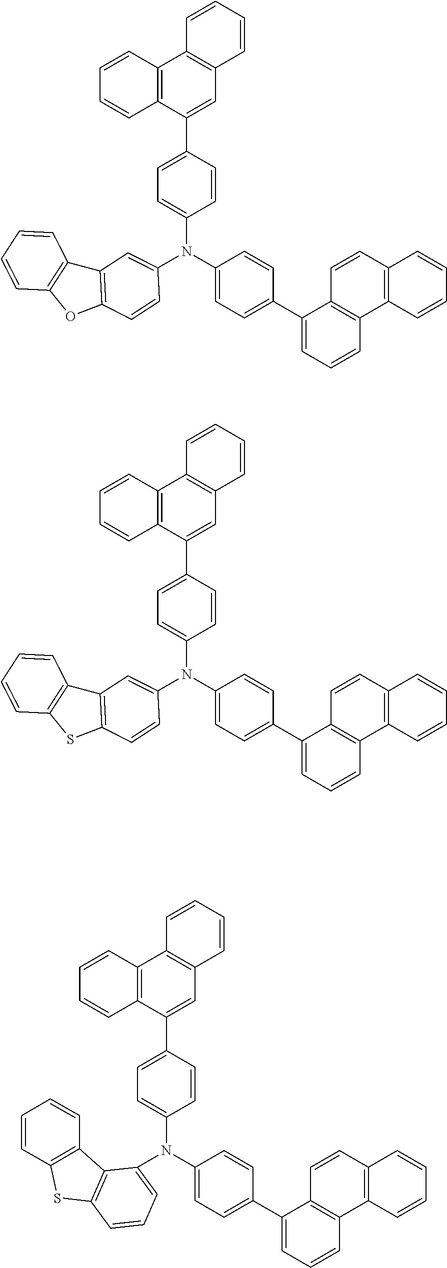 [SMILES: c1ccc2c(c1)ccc1c(-c3ccc(N(c4ccc(-c5cc6ccccc6c6ccccc56)cc4)c4ccc5oc6ccccc6c5c4)cc3)cccc12 ; c1ccc2c(c1)ccc1c(-c3ccc(N(c4ccc(-c5cc6ccccc6c6ccccc56)cc4)c4ccc5sc6ccccc6c5c4)cc3)cccc12 ; c1ccc2c(c1)ccc1c(-c3ccc(N(c4ccc(-c5cc6ccccc6c6ccccc56)cc4)c4cccc5sc6ccccc6c45)cc3)cccc12]

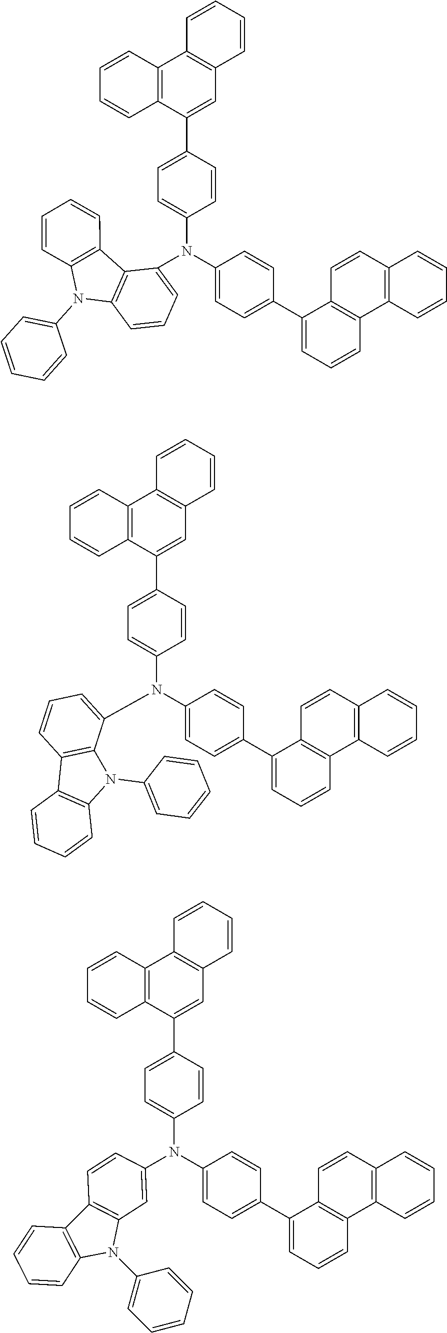 [SMILES: c1ccc(-n2c3ccccc3c3c(N(c4ccc(-c5cccc6c5ccc5ccccc56)cc4)c4ccc(-c5cc6ccccc6c6ccccc56)cc4)cccc32)cc1 ; c1ccc(-n2c3ccccc3c3cccc(N(c4ccc(-c5cccc6c5ccc5ccccc56)cc4)c4ccc(-c5cc6ccccc6c6ccccc56)cc4)c32)cc1 ; c1ccc(-n2c3ccccc3c3ccc(N(c4ccc(-c5cccc6c5ccc5ccccc56)cc4)c4ccc(-c5cc6ccccc6c6ccccc56)cc4)cc32)cc1]

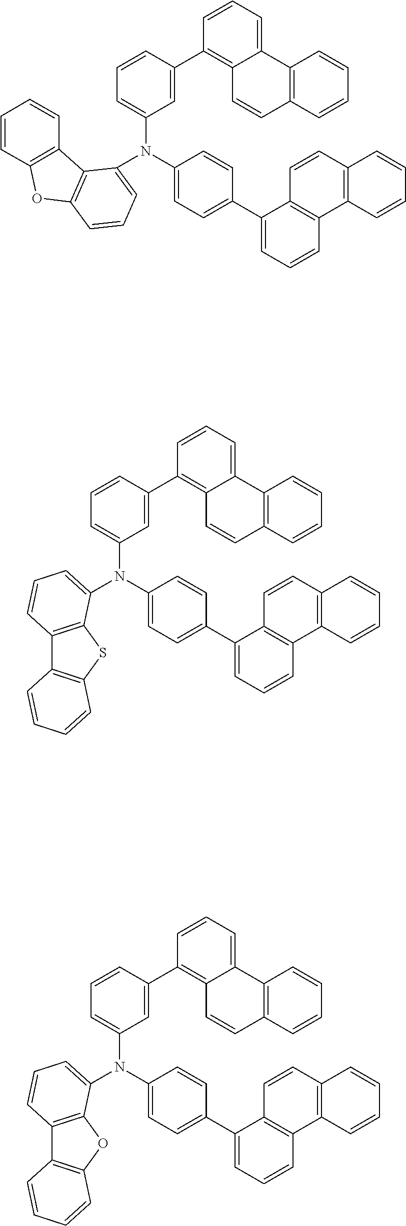 [SMILES: c1cc(-c2cccc3c2ccc2ccccc23)cc(N(c2ccc(-c3cccc4c3ccc3ccccc34)cc2)c2cccc3oc4ccccc4c23)c1 ; c1cc(-c2cccc3c2ccc2ccccc23)cc(N(c2ccc(-c3cccc4c3ccc3ccccc34)cc2)c2cccc3c2sc2ccccc23)c1 ; c1cc(-c2cccc3c2ccc2ccccc23)cc(N(c2ccc(-c3cccc4c3ccc3ccccc34)cc2)c2cccc3c2oc2ccccc23)c1]

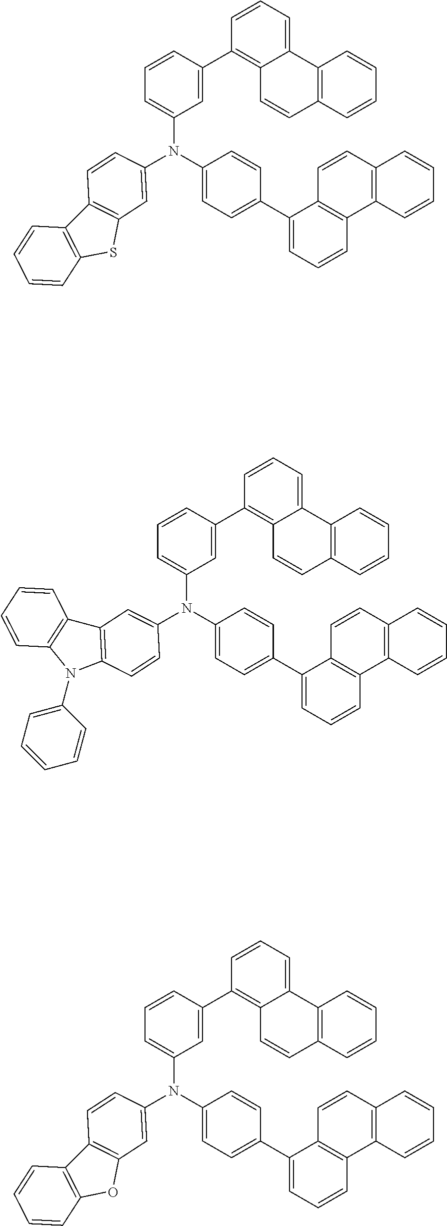 [SMILES: c1cc(-c2cccc3c2ccc2ccccc23)cc(N(c2ccc(-c3cccc4c3ccc3ccccc34)cc2)c2ccc3c(c2)sc2ccccc23)c1 ; c1ccc(-n2c3ccccc3c3cc(N(c4ccc(-c5cccc6c5ccc5ccccc56)cc4)c4cccc(-c5cccc6c5ccc5ccccc56)c4)ccc32)cc1 ; c1cc(-c2cccc3c2ccc2ccccc23)cc(N(c2ccc(-c3cccc4c3ccc3ccccc34)cc2)c2ccc3c(c2)oc2ccccc23)c1]

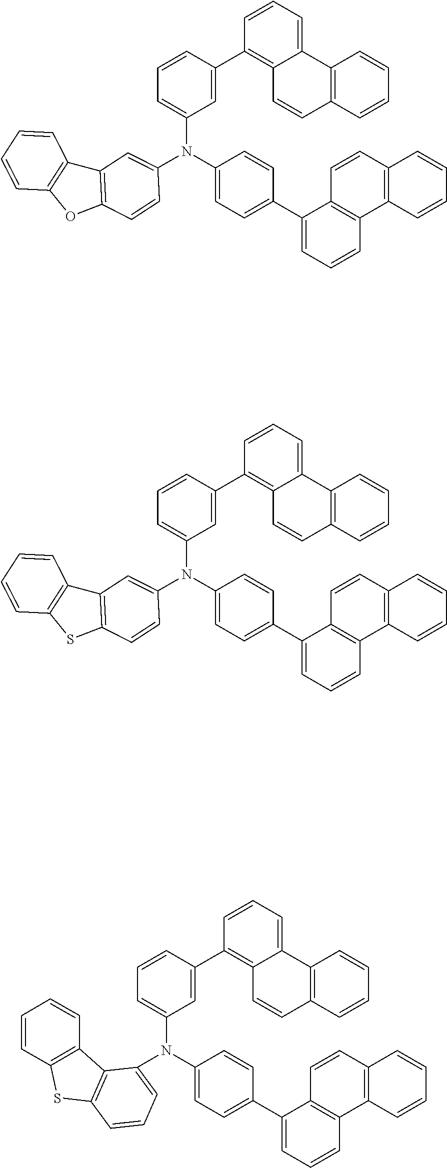 [SMILES: c1cc(-c2cccc3c2ccc2ccccc23)cc(N(c2ccc(-c3cccc4c3ccc3ccccc34)cc2)c2ccc3oc4ccccc4c3c2)c1 ; c1cc(-c2cccc3c2ccc2ccccc23)cc(N(c2ccc(-c3cccc4c3ccc3ccccc34)cc2)c2ccc3sc4ccccc4c3c2)c1 ; c1cc(-c2cccc3c2ccc2ccccc23)cc(N(c2ccc(-c3cccc4c3ccc3ccccc34)cc2)c2cccc3sc4ccccc4c23)c1]

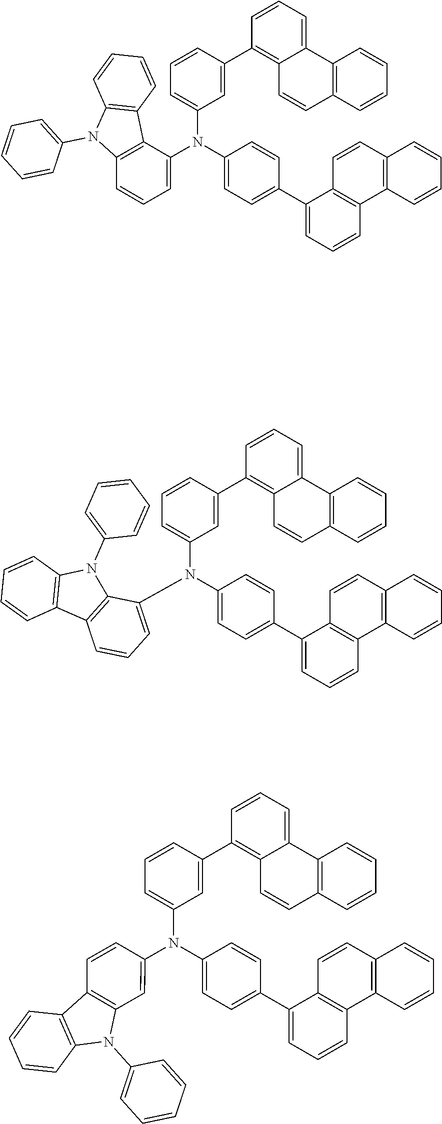 [SMILES: c1ccc(-n2c3ccccc3c3c(N(c4ccc(-c5cccc6c5ccc5ccccc56)cc4)c4cccc(-c5cccc6c5ccc5ccccc56)c4)cccc32)cc1 ; c1ccc(-n2c3ccccc3c3cccc(N(c4ccc(-c5cccc6c5ccc5ccccc56)cc4)c4cccc(-c5cccc6c5ccc5ccccc56)c4)c32)cc1 ; c1ccc(-n2c3ccccc3c3ccc(N(c4ccc(-c5cccc6c5ccc5ccccc56)cc4)c4cccc(-c5cccc6c5ccc5ccccc56)c4)cc32)cc1]

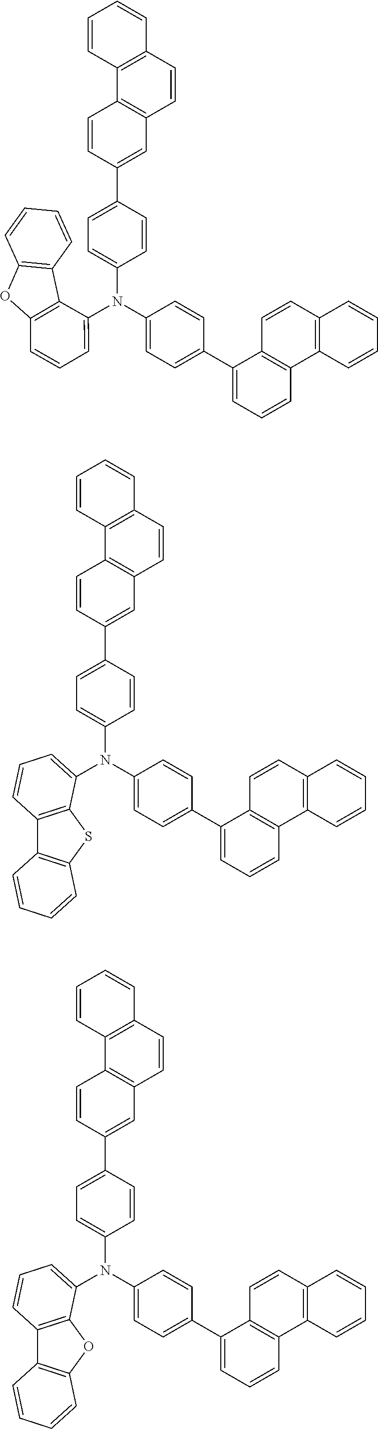 [SMILES: c1ccc2c(c1)ccc1cc(-c3ccc(N(c4ccc(-c5cccc6c5ccc5ccccc56)cc4)c4cccc5oc6ccccc6c45)cc3)ccc12 ; c1ccc2c(c1)ccc1cc(-c3ccc(N(c4ccc(-c5cccc6c5ccc5ccccc56)cc4)c4cccc5c4sc4ccccc45)cc3)ccc12 ; c1ccc2c(c1)ccc1cc(-c3ccc(N(c4ccc(-c5cccc6c5ccc5ccccc56)cc4)c4cccc5c4oc4ccccc45)cc3)ccc12]

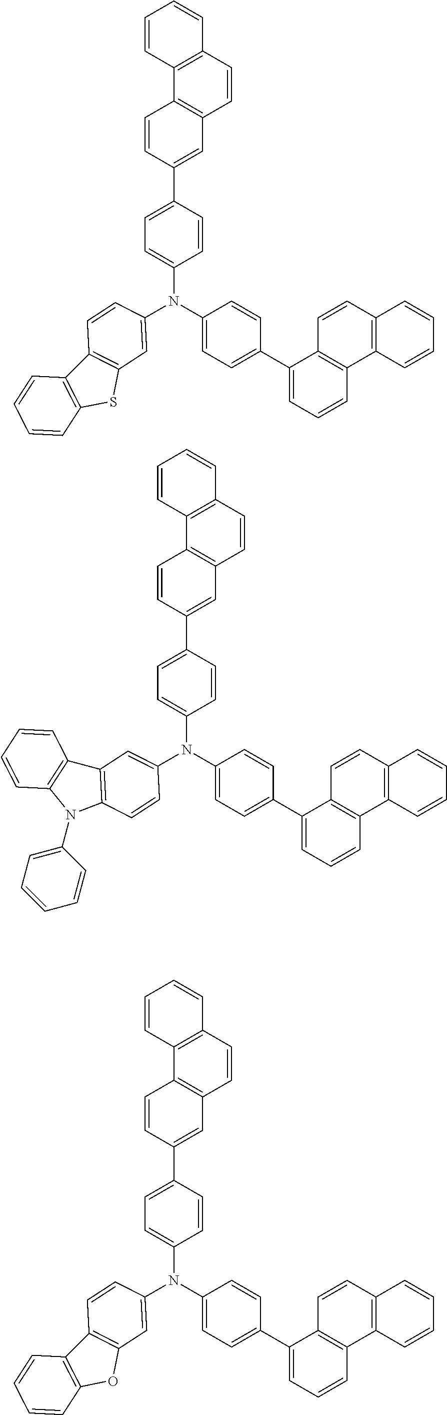 [SMILES: c1ccc2c(c1)ccc1cc(-c3ccc(N(c4ccc(-c5cccc6c5ccc5ccccc56)cc4)c4ccc5c(c4)sc4ccccc45)cc3)ccc12 ; c1ccc(-n2c3ccccc3c3cc(N(c4ccc(-c5ccc6c(ccc7ccccc76)c5)cc4)c4ccc(-c5cccc6c5ccc5ccccc56)cc4)ccc32)cc1 ; c1ccc2c(c1)ccc1cc(-c3ccc(N(c4ccc(-c5cccc6c5ccc5ccccc56)cc4)c4ccc5c(c4)oc4ccccc45)cc3)ccc12]

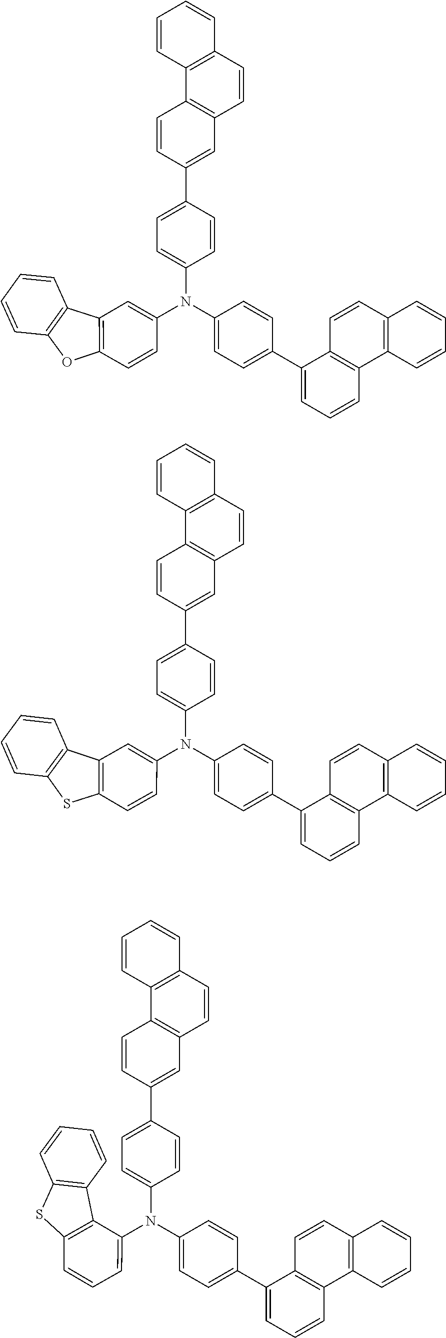 [SMILES: c1ccc2c(c1)ccc1cc(-c3ccc(N(c4ccc(-c5cccc6c5ccc5ccccc56)cc4)c4ccc5oc6ccccc6c5c4)cc3)ccc12 ; c1ccc2c(c1)ccc1cc(-c3ccc(N(c4ccc(-c5cccc6c5ccc5ccccc56)cc4)c4ccc5sc6ccccc6c5c4)cc3)ccc12 ; c1ccc2c(c1)ccc1cc(-c3ccc(N(c4ccc(-c5cccc6c5ccc5ccccc56)cc4)c4cccc5sc6ccccc6c45)cc3)ccc12]

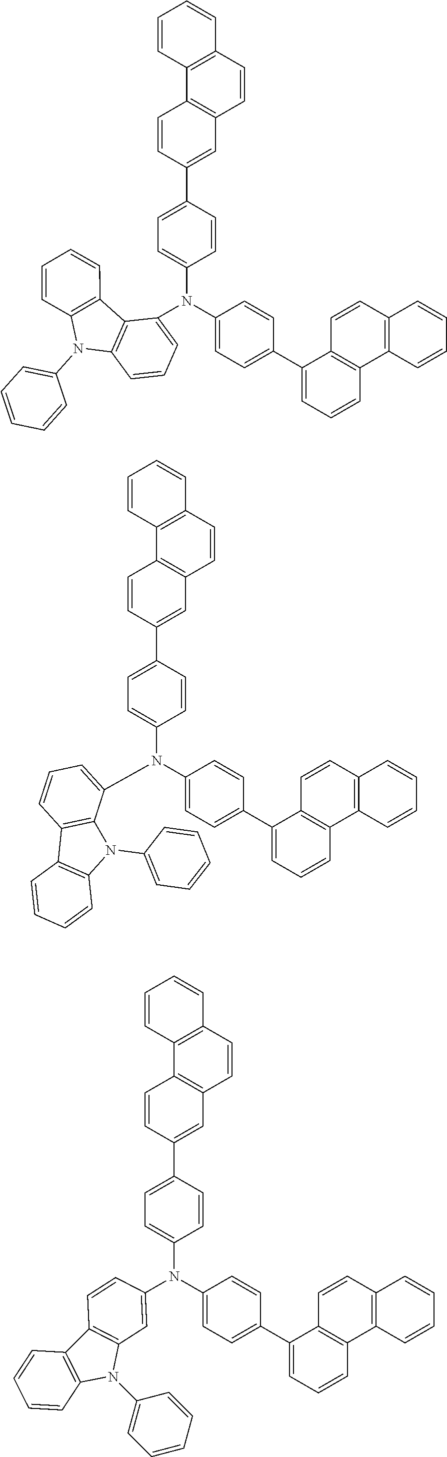 [SMILES: c1ccc(-n2c3ccccc3c3c(N(c4ccc(-c5ccc6c(ccc7ccccc76)c5)cc4)c4ccc(-c5cccc6c5ccc5ccccc56)cc4)cccc32)cc1 ; c1ccc(-n2c3ccccc3c3cccc(N(c4ccc(-c5ccc6c(ccc7ccccc76)c5)cc4)c4ccc(-c5cccc6c5ccc5ccccc56)cc4)c32)cc1 ; c1ccc(-n2c3ccccc3c3ccc(N(c4ccc(-c5ccc6c(ccc7ccccc76)c5)cc4)c4ccc(-c5cccc6c5ccc5ccccc56)cc4)cc32)cc1]

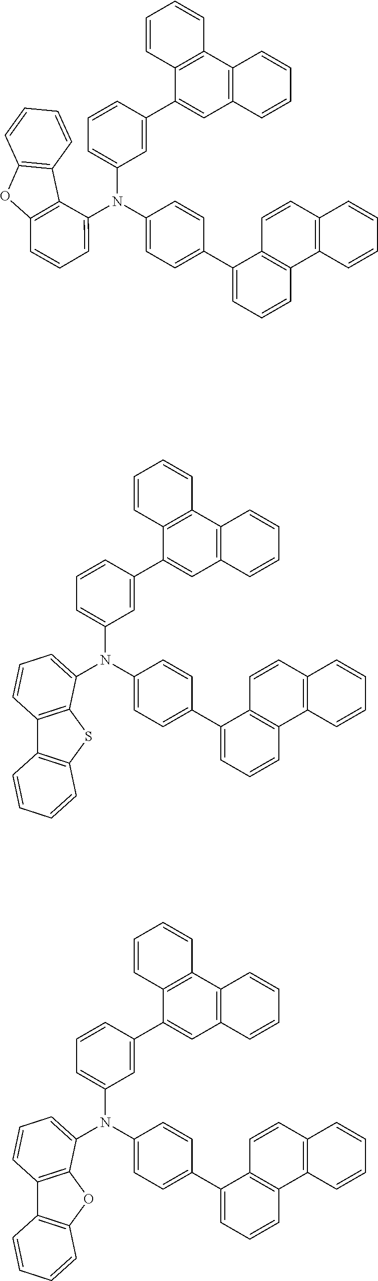 [SMILES: c1cc(-c2cc3ccccc3c3ccccc23)cc(N(c2ccc(-c3cccc4c3ccc3ccccc34)cc2)c2cccc3oc4ccccc4c23)c1 ; c1cc(-c2cc3ccccc3c3ccccc23)cc(N(c2ccc(-c3cccc4c3ccc3ccccc34)cc2)c2cccc3c2sc2ccccc23)c1 ; c1cc(-c2cc3ccccc3c3ccccc23)cc(N(c2ccc(-c3cccc4c3ccc3ccccc34)cc2)c2cccc3c2oc2ccccc23)c1]

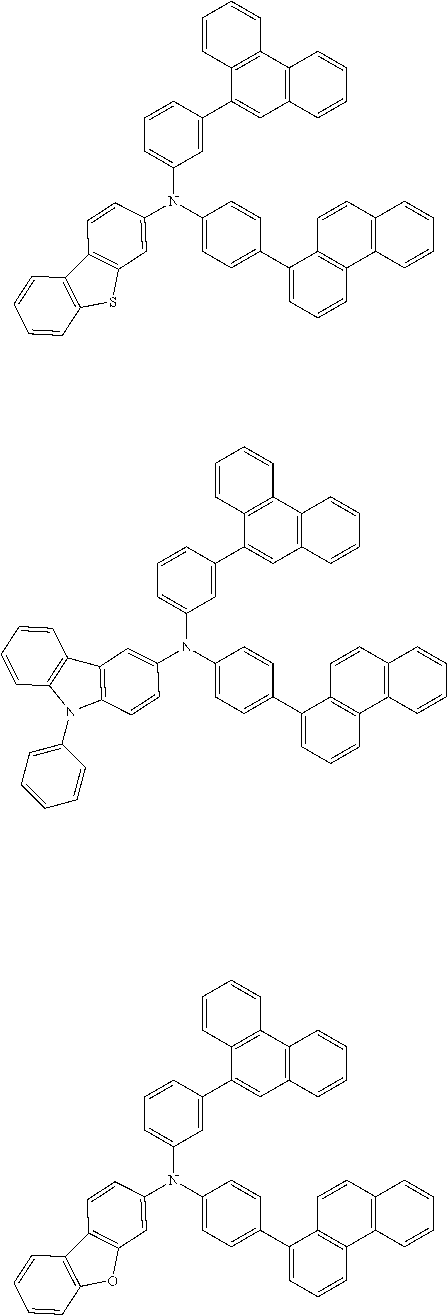 [SMILES: c1cc(-c2cc3ccccc3c3ccccc23)cc(N(c2ccc(-c3cccc4c3ccc3ccccc34)cc2)c2ccc3c(c2)sc2ccccc23)c1 ; c1ccc(-n2c3ccccc3c3cc(N(c4ccc(-c5cccc6c5ccc5ccccc56)cc4)c4cccc(-c5cc6ccccc6c6ccccc56)c4)ccc32)cc1 ; c1cc(-c2cc3ccccc3c3ccccc23)cc(N(c2ccc(-c3cccc4c3ccc3ccccc34)cc2)c2ccc3c(c2)oc2ccccc23)c1]

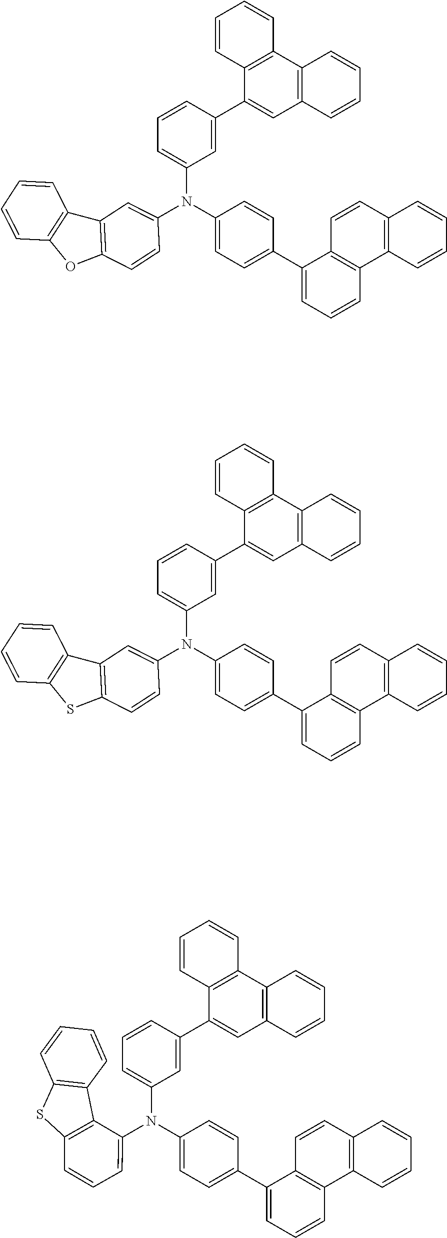 [SMILES: c1cc(-c2cc3ccccc3c3ccccc23)cc(N(c2ccc(-c3cccc4c3ccc3ccccc34)cc2)c2ccc3oc4ccccc4c3c2)c1 ; c1cc(-c2cc3ccccc3c3ccccc23)cc(N(c2ccc(-c3cccc4c3ccc3ccccc34)cc2)c2ccc3sc4ccccc4c3c2)c1 ; c1cc(-c2cc3ccccc3c3ccccc23)cc(N(c2ccc(-c3cccc4c3ccc3ccccc34)cc2)c2cccc3sc4ccccc4c23)c1]

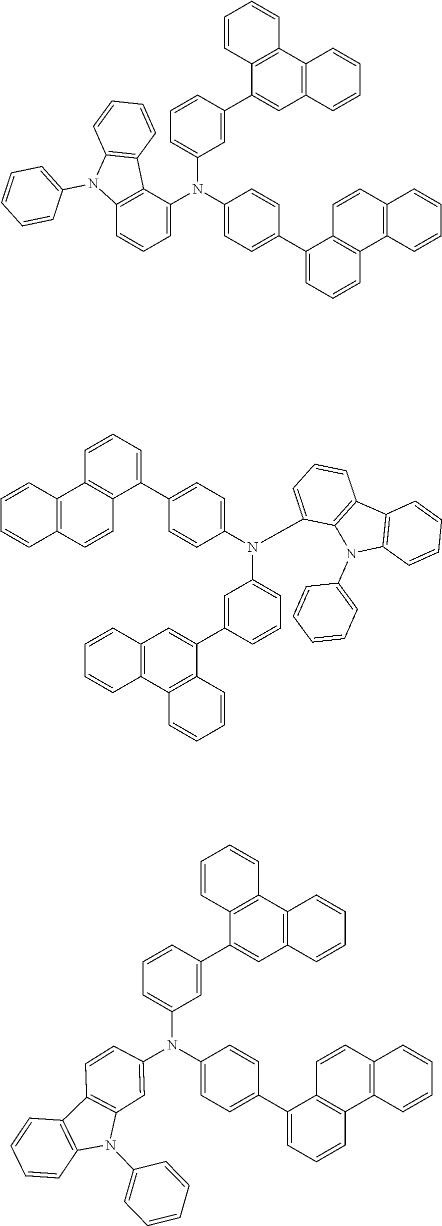 [SMILES: c1ccc(-n2c3ccccc3c3c(N(c4ccc(-c5cccc6c5ccc5ccccc56)cc4)c4cccc(-c5cc6ccccc6c6ccccc56)c4)cccc32)cc1 ; c1ccc(-n2c3ccccc3c3cccc(N(c4ccc(-c5cccc6c5ccc5ccccc56)cc4)c4cccc(-c5cc6ccccc6c6ccccc56)c4)c32)cc1 ; c1ccc(-n2c3ccccc3c3ccc(N(c4ccc(-c5cccc6c5ccc5ccccc56)cc4)c4cccc(-c5cc6ccccc6c6ccccc56)c4)cc32)cc1]

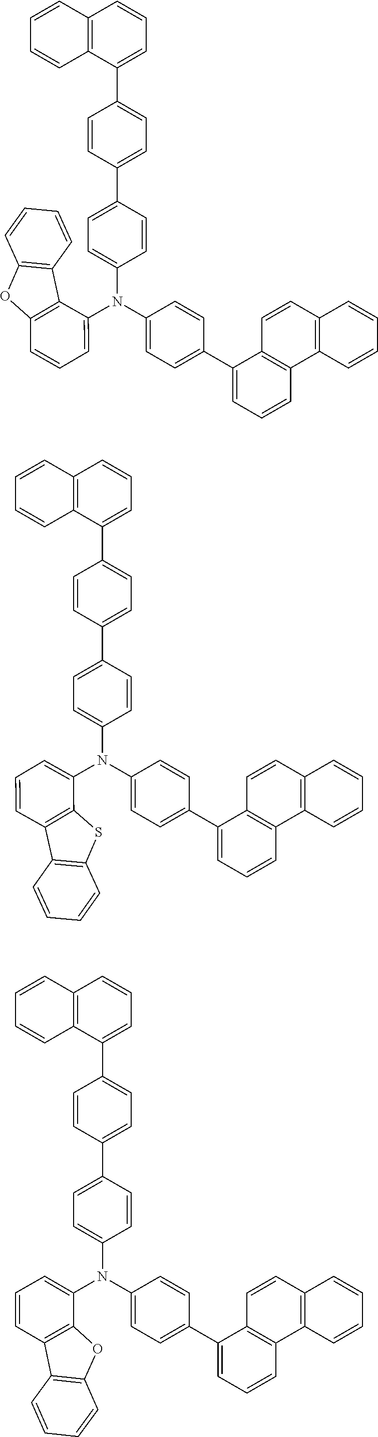 [SMILES: c1ccc2c(-c3ccc(-c4ccc(N(c5ccc(-c6cccc7c6ccc6ccccc67)cc5)c5cccc6oc7ccccc7c56)cc4)cc3)cccc2c1 ; c1ccc2c(-c3ccc(-c4ccc(N(c5ccc(-c6cccc7c6ccc6ccccc67)cc5)c5cccc6c5sc5ccccc56)cc4)cc3)cccc2c1 ; c1ccc2c(-c3ccc(-c4ccc(N(c5ccc(-c6cccc7c6ccc6ccccc67)cc5)c5cccc6c5oc5ccccc56)cc4)cc3)cccc2c1]

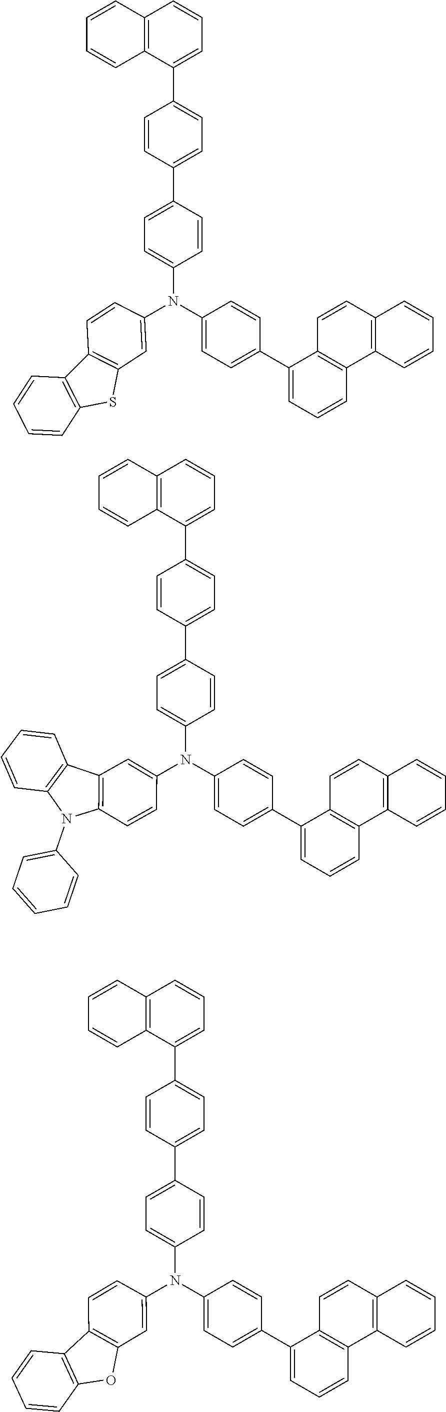 [SMILES: c1ccc2c(-c3ccc(-c4ccc(N(c5ccc(-c6cccc7c6ccc6ccccc67)cc5)c5ccc6c(c5)sc5ccccc56)cc4)cc3)cccc2c1 ; c1ccc(-n2c3ccccc3c3cc(N(c4ccc(-c5ccc(-c6cccc7ccccc67)cc5)cc4)c4ccc(-c5cccc6c5ccc5ccccc56)cc4)ccc32)cc1 ; c1ccc2c(-c3ccc(-c4ccc(N(c5ccc(-c6cccc7c6ccc6ccccc67)cc5)c5ccc6c(c5)oc5ccccc56)cc4)cc3)cccc2c1]

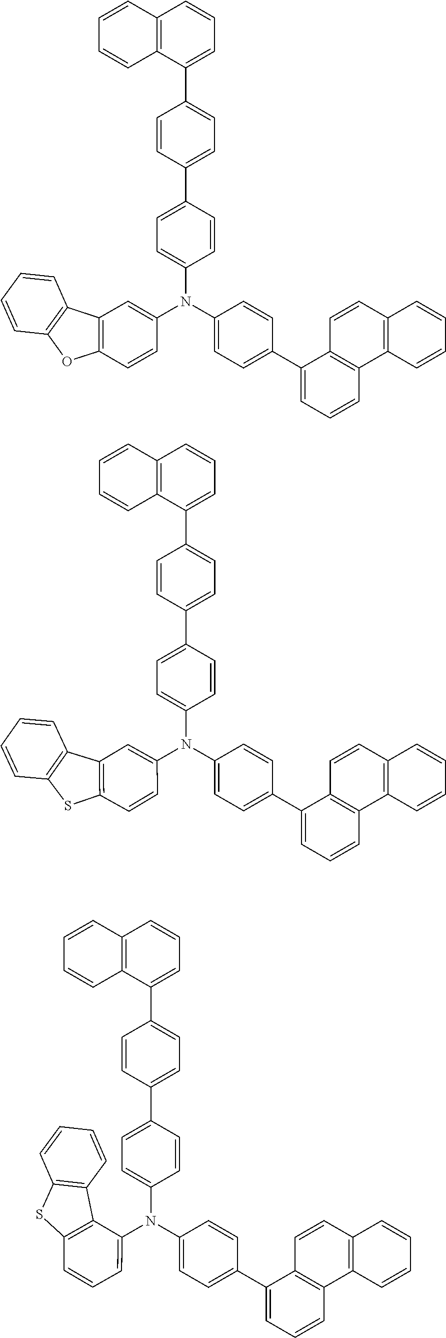 [SMILES: c1ccc2c(-c3ccc(-c4ccc(N(c5ccc(-c6cccc7c6ccc6ccccc67)cc5)c5ccc6oc7ccccc7c6c5)cc4)cc3)cccc2c1 ; c1ccc2c(-c3ccc(-c4ccc(N(c5ccc(-c6cccc7c6ccc6ccccc67)cc5)c5ccc6sc7ccccc7c6c5)cc4)cc3)cccc2c1 ; c1ccc2c(-c3ccc(-c4ccc(N(c5ccc(-c6cccc7c6ccc6ccccc67)cc5)c5cccc6sc7ccccc7c56)cc4)cc3)cccc2c1]

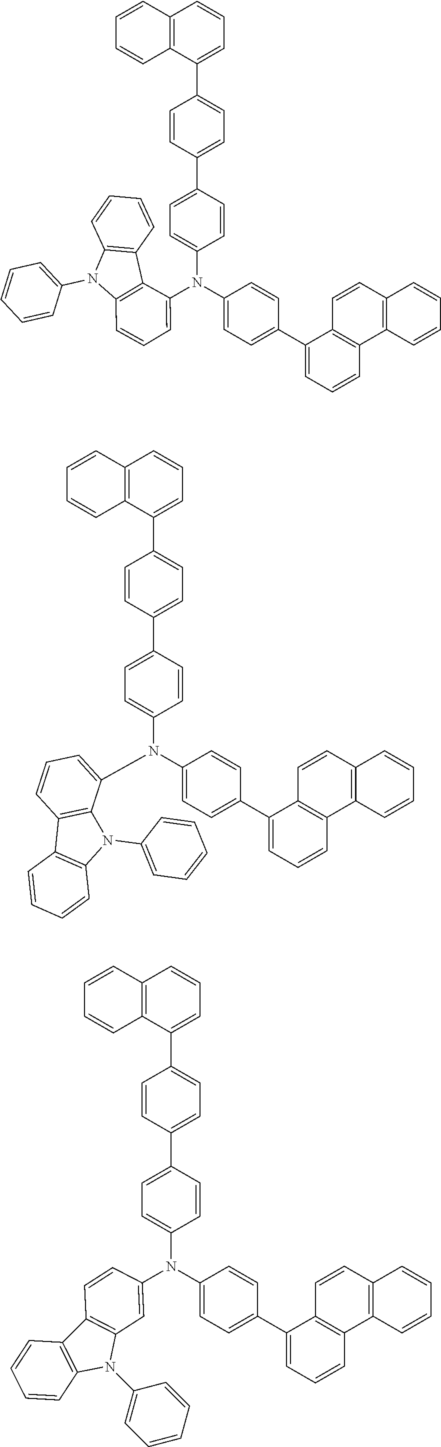 [SMILES: c1ccc(-n2c3ccccc3c3c(N(c4ccc(-c5ccc(-c6cccc7ccccc67)cc5)cc4)c4ccc(-c5cccc6c5ccc5ccccc56)cc4)cccc32)cc1 ; c1ccc(-n2c3ccccc3c3cccc(N(c4ccc(-c5ccc(-c6cccc7ccccc67)cc5)cc4)c4ccc(-c5cccc6c5ccc5ccccc56)cc4)c32)cc1 ; c1ccc(-n2c3ccccc3c3ccc(N(c4ccc(-c5ccc(-c6cccc7ccccc67)cc5)cc4)c4ccc(-c5cccc6c5ccc5ccccc56)cc4)cc32)cc1]

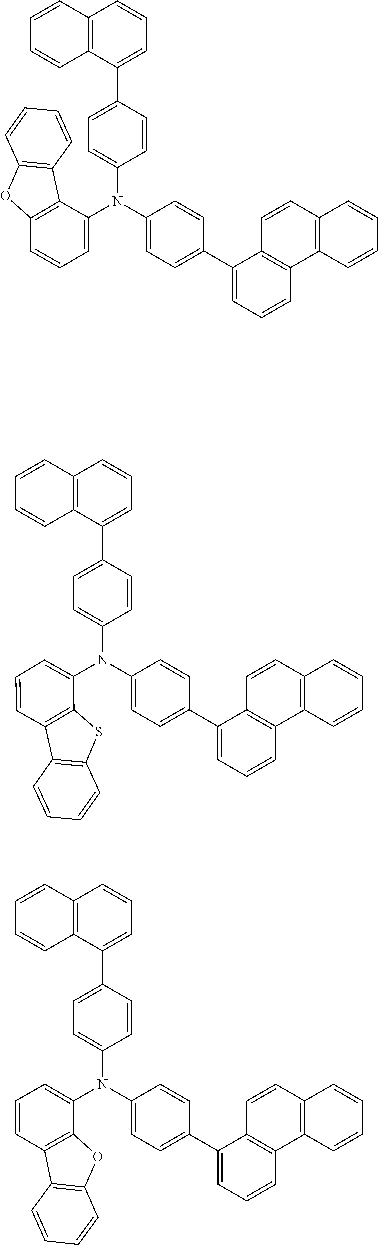 [SMILES: c1ccc2c(-c3ccc(N(c4ccc(-c5cccc6c5ccc5ccccc56)cc4)c4cccc5oc6ccccc6c45)cc3)cccc2c1 ; c1ccc2c(-c3ccc(N(c4ccc(-c5cccc6c5ccc5ccccc56)cc4)c4cccc5c4sc4ccccc45)cc3)cccc2c1 ; c1ccc2c(-c3ccc(N(c4ccc(-c5cccc6c5ccc5ccccc56)cc4)c4cccc5c4oc4ccccc45)cc3)cccc2c1]

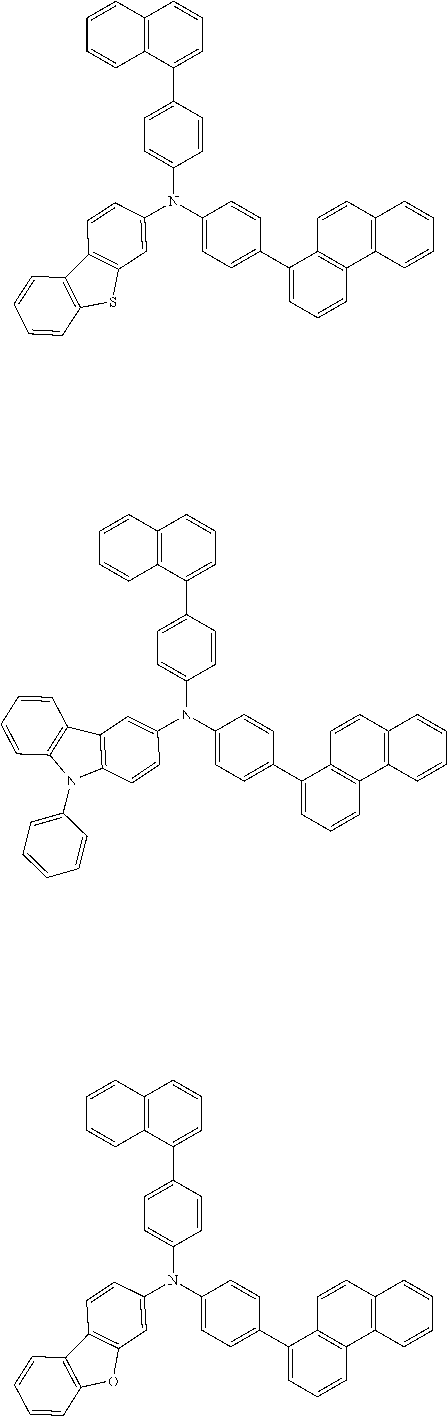 [SMILES: c1ccc2c(-c3ccc(N(c4ccc(-c5cccc6c5ccc5ccccc56)cc4)c4ccc5c(c4)sc4ccccc45)cc3)cccc2c1 ; c1ccc(-n2c3ccccc3c3cc(N(c4ccc(-c5cccc6ccccc56)cc4)c4ccc(-c5cccc6c5ccc5ccccc56)cc4)ccc32)cc1 ; c1ccc2c(-c3ccc(N(c4ccc(-c5cccc6c5ccc5ccccc56)cc4)c4ccc5c(c4)oc4ccccc45)cc3)cccc2c1]

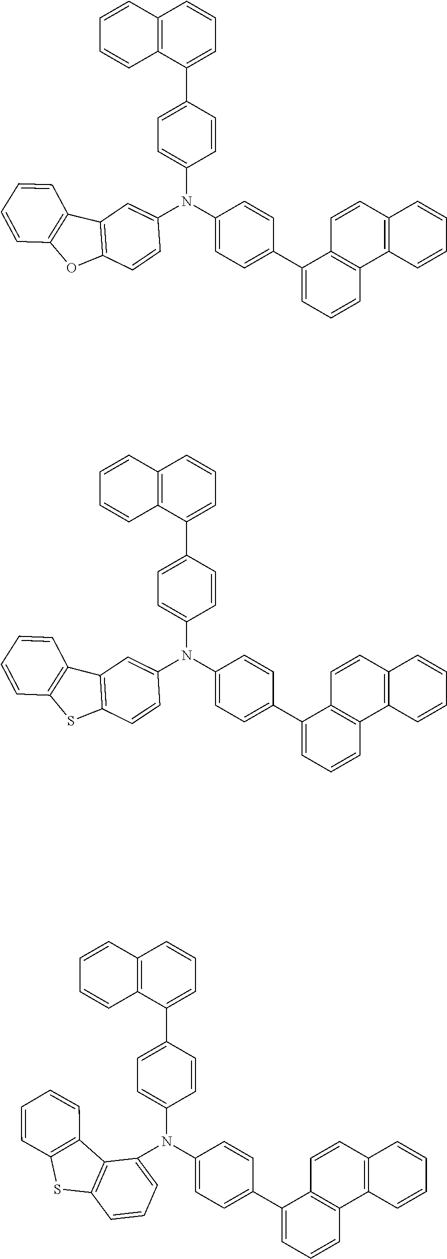 [SMILES: c1ccc2c(-c3ccc(N(c4ccc(-c5cccc6c5ccc5ccccc56)cc4)c4ccc5oc6ccccc6c5c4)cc3)cccc2c1 ; c1ccc2c(-c3ccc(N(c4ccc(-c5cccc6c5ccc5ccccc56)cc4)c4ccc5sc6ccccc6c5c4)cc3)cccc2c1 ; c1ccc2c(-c3ccc(N(c4ccc(-c5cccc6c5ccc5ccccc56)cc4)c4cccc5sc6ccccc6c45)cc3)cccc2c1]

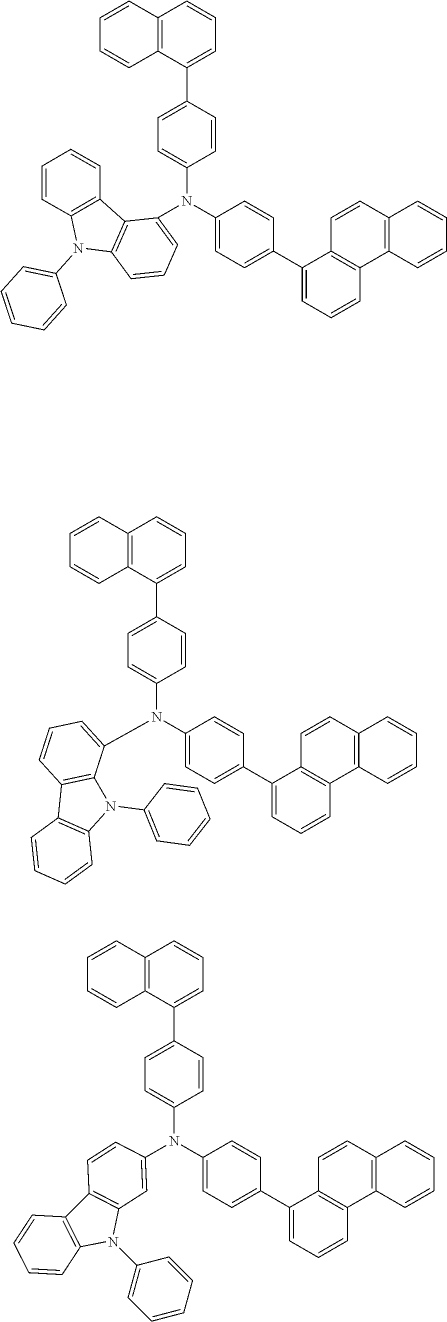 [SMILES: c1ccc(-n2c3ccccc3c3c(N(c4ccc(-c5cccc6ccccc56)cc4)c4ccc(-c5cccc6c5ccc5ccccc56)cc4)cccc32)cc1 ; c1ccc(-n2c3ccccc3c3cccc(N(c4ccc(-c5cccc6ccccc56)cc4)c4ccc(-c5cccc6c5ccc5ccccc56)cc4)c32)cc1 ; c1ccc(-n2c3ccccc3c3ccc(N(c4ccc(-c5cccc6ccccc56)cc4)c4ccc(-c5cccc6c5ccc5ccccc56)cc4)cc32)cc1]

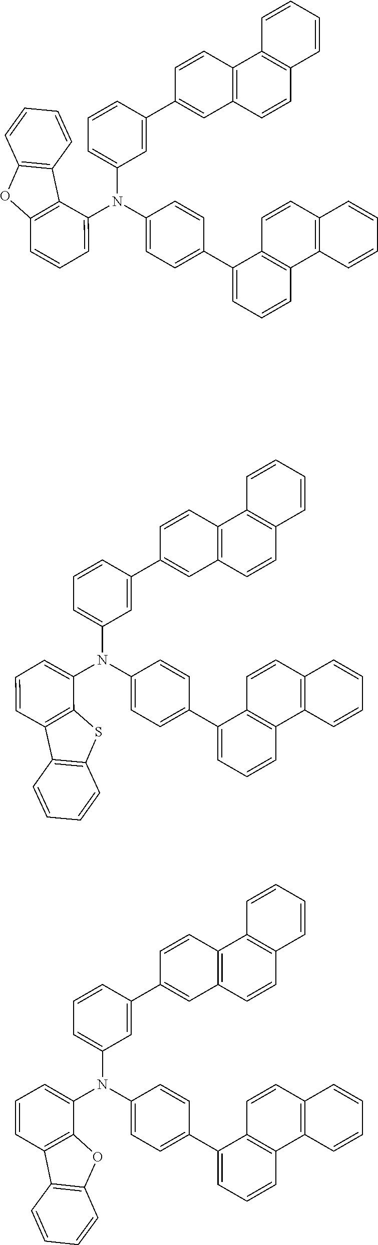 [SMILES: c1cc(-c2ccc3c(ccc4ccccc43)c2)cc(N(c2ccc(-c3cccc4c3ccc3ccccc34)cc2)c2cccc3oc4ccccc4c23)c1 ; c1cc(-c2ccc3c(ccc4ccccc43)c2)cc(N(c2ccc(-c3cccc4c3ccc3ccccc34)cc2)c2cccc3c2sc2ccccc23)c1 ; c1cc(-c2ccc3c(ccc4ccccc43)c2)cc(N(c2ccc(-c3cccc4c3ccc3ccccc34)cc2)c2cccc3c2oc2ccccc23)c1]

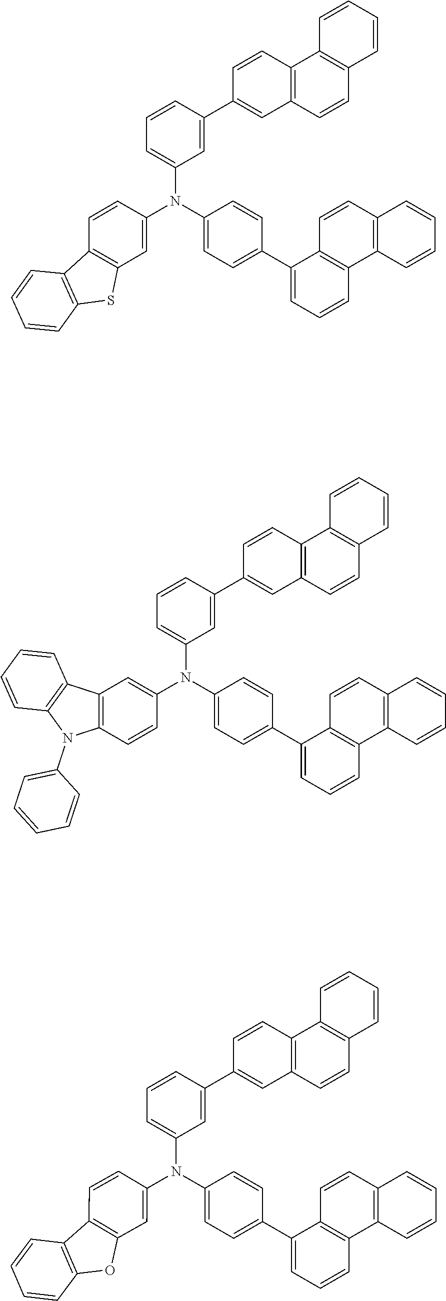 [SMILES: c1cc(-c2ccc3c(ccc4ccccc43)c2)cc(N(c2ccc(-c3cccc4c3ccc3ccccc34)cc2)c2ccc3c(c2)sc2ccccc23)c1 ; c1ccc(-n2c3ccccc3c3cc(N(c4ccc(-c5cccc6c5ccc5ccccc56)cc4)c4cccc(-c5ccc6c(ccc7ccccc76)c5)c4)ccc32)cc1 ; c1cc(-c2ccc3c(ccc4ccccc43)c2)cc(N(c2ccc(-c3cccc4c3ccc3ccccc34)cc2)c2ccc3c(c2)oc2ccccc23)c1]

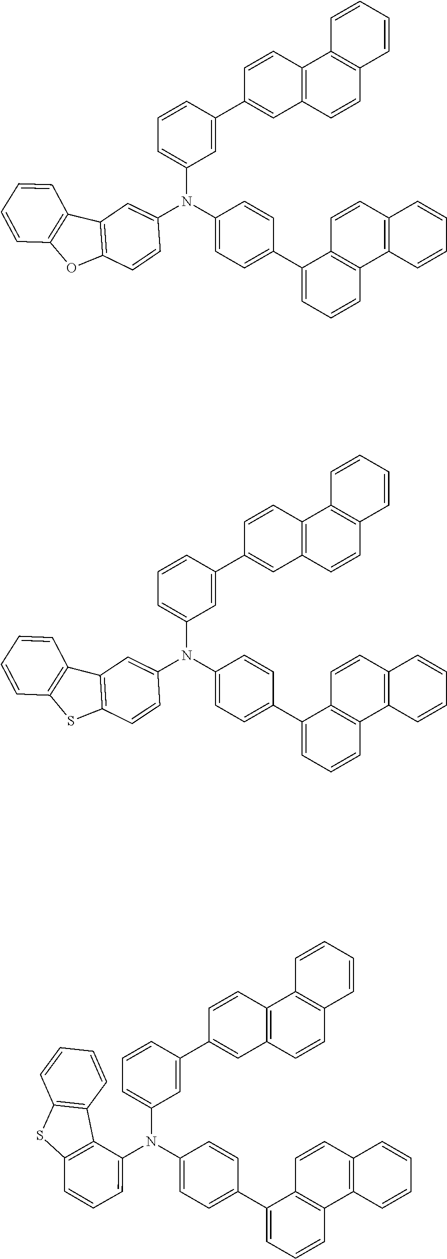 [SMILES: c1cc(-c2ccc3c(ccc4ccccc43)c2)cc(N(c2ccc(-c3cccc4c3ccc3ccccc34)cc2)c2ccc3oc4ccccc4c3c2)c1 ; c1cc(-c2ccc3c(ccc4ccccc43)c2)cc(N(c2ccc(-c3cccc4c3ccc3ccccc34)cc2)c2ccc3sc4ccccc4c3c2)c1 ; c1cc(-c2ccc3c(ccc4ccccc43)c2)cc(N(c2ccc(-c3cccc4c3ccc3ccccc34)cc2)c2cccc3sc4ccccc4c23)c1]

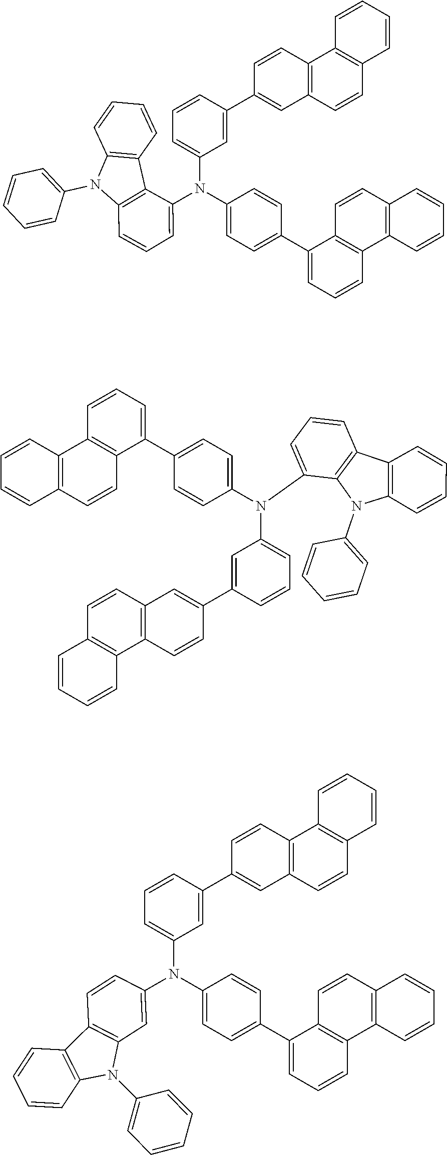 [SMILES: c1ccc(-n2c3ccccc3c3c(N(c4ccc(-c5cccc6c5ccc5ccccc56)cc4)c4cccc(-c5ccc6c(ccc7ccccc76)c5)c4)cccc32)cc1 ; c1ccc(-n2c3ccccc3c3cccc(N(c4ccc(-c5cccc6c5ccc5ccccc56)cc4)c4cccc(-c5ccc6c(ccc7ccccc76)c5)c4)c32)cc1 ; c1ccc(-n2c3ccccc3c3ccc(N(c4ccc(-c5cccc6c5ccc5ccccc56)cc4)c4cccc(-c5ccc6c(ccc7ccccc76)c5)c4)cc32)cc1]

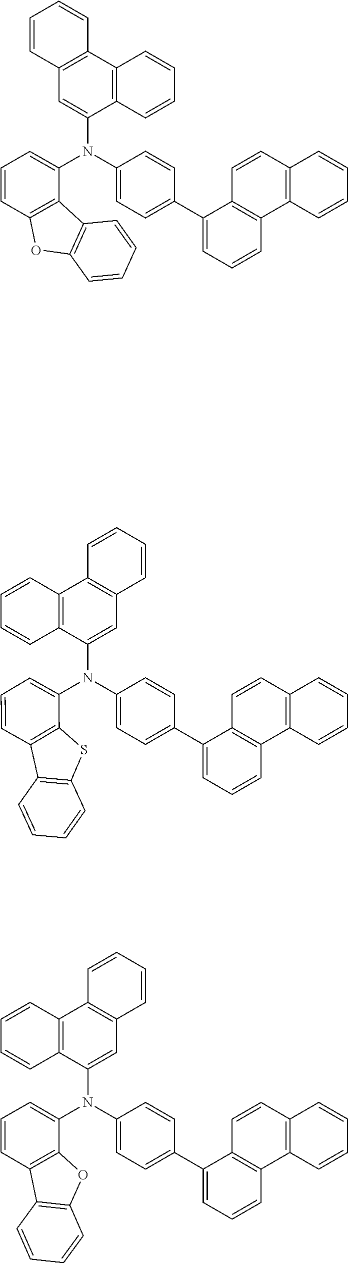 [SMILES: c1ccc2c(c1)ccc1c(-c3ccc(N(c4cc5ccccc5c5ccccc45)c4cccc5oc6ccccc6c45)cc3)cccc12 ; c1ccc2c(c1)ccc1c(-c3ccc(N(c4cc5ccccc5c5ccccc45)c4cccc5c4sc4ccccc45)cc3)cccc12 ; c1ccc2c(c1)ccc1c(-c3ccc(N(c4cc5ccccc5c5ccccc45)c4cccc5c4oc4ccccc45)cc3)cccc12]

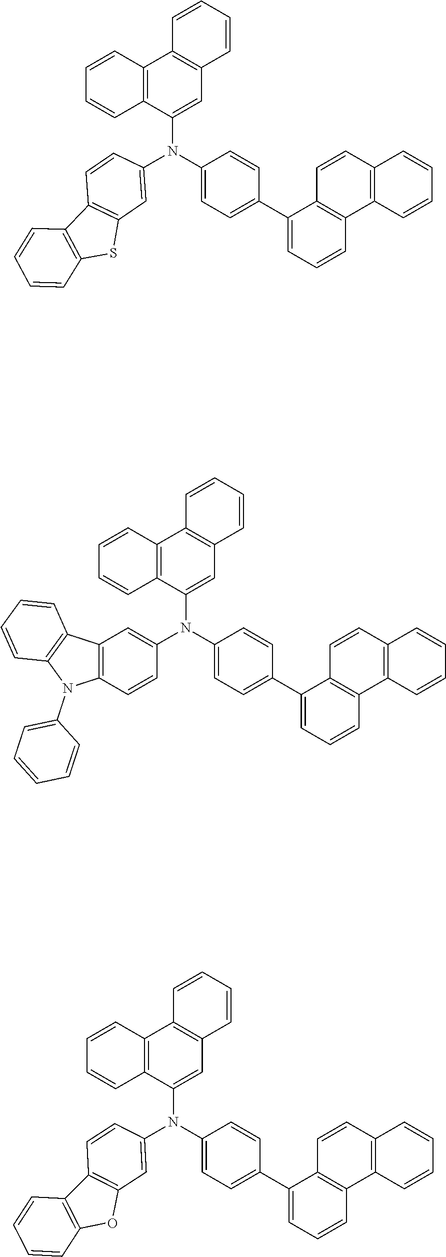 [SMILES: c1ccc2c(c1)ccc1c(-c3ccc(N(c4ccc5c(c4)sc4ccccc45)c4cc5ccccc5c5ccccc45)cc3)cccc12 ; c1ccc(-n2c3ccccc3c3cc(N(c4ccc(-c5cccc6c5ccc5ccccc56)cc4)c4cc5ccccc5c5ccccc45)ccc32)cc1 ; c1ccc2c(c1)ccc1c(-c3ccc(N(c4ccc5c(c4)oc4ccccc45)c4cc5ccccc5c5ccccc45)cc3)cccc12]

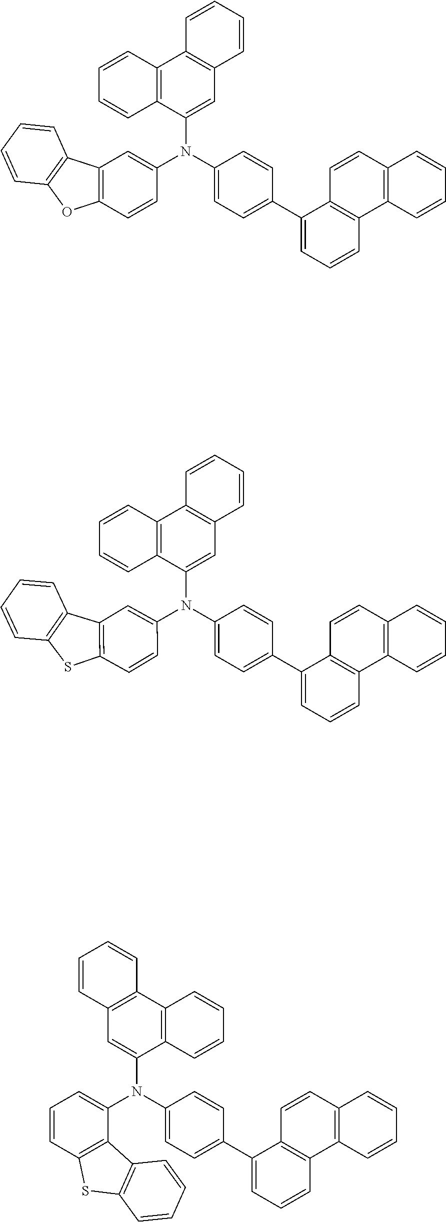 [SMILES: c1ccc2c(c1)ccc1c(-c3ccc(N(c4ccc5oc6ccccc6c5c4)c4cc5ccccc5c5ccccc45)cc3)cccc12 ; c1ccc2c(c1)ccc1c(-c3ccc(N(c4ccc5sc6ccccc6c5c4)c4cc5ccccc5c5ccccc45)cc3)cccc12 ; c1ccc2c(c1)ccc1c(-c3ccc(N(c4cc5ccccc5c5ccccc45)c4cccc5sc6ccccc6c45)cc3)cccc12]

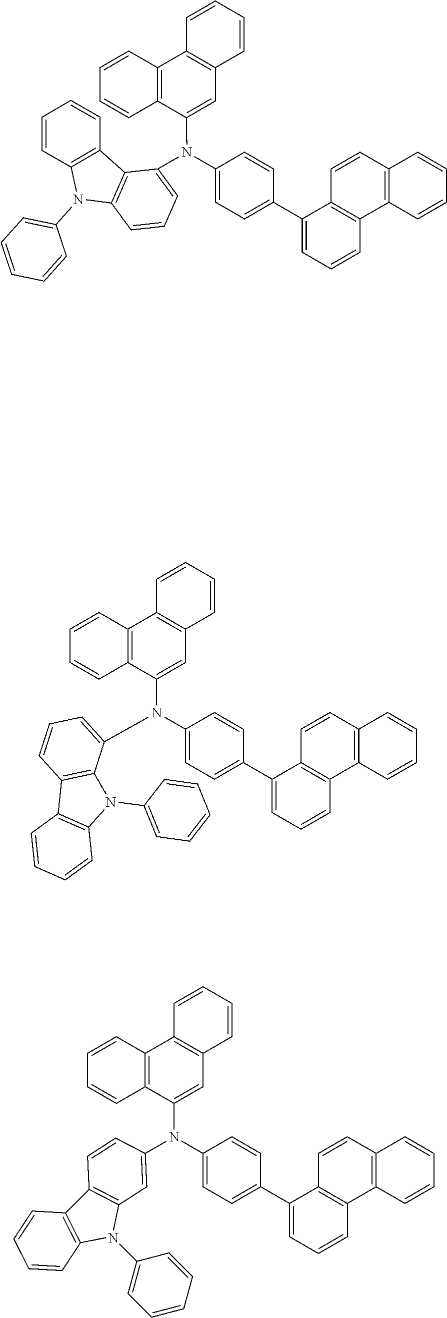 [SMILES: c1ccc(-n2c3ccccc3c3c(N(c4ccc(-c5cccc6c5ccc5ccccc56)cc4)c4cc5ccccc5c5ccccc45)cccc32)cc1 ; c1ccc(-n2c3ccccc3c3cccc(N(c4ccc(-c5cccc6c5ccc5ccccc56)cc4)c4cc5ccccc5c5ccccc45)c32)cc1 ; c1ccc(-n2c3ccccc3c3ccc(N(c4ccc(-c5cccc6c5ccc5ccccc56)cc4)c4cc5ccccc5c5ccccc45)cc32)cc1]

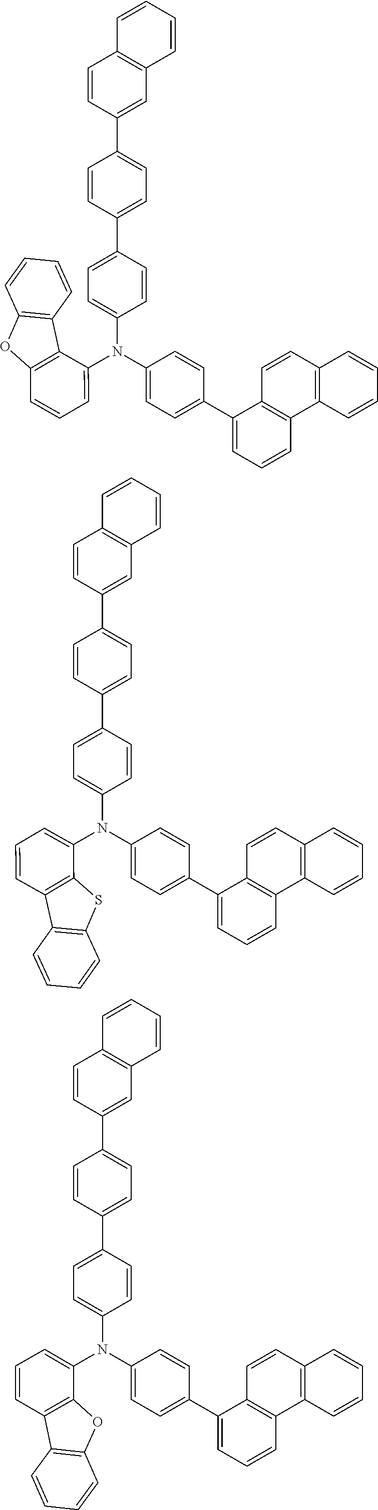

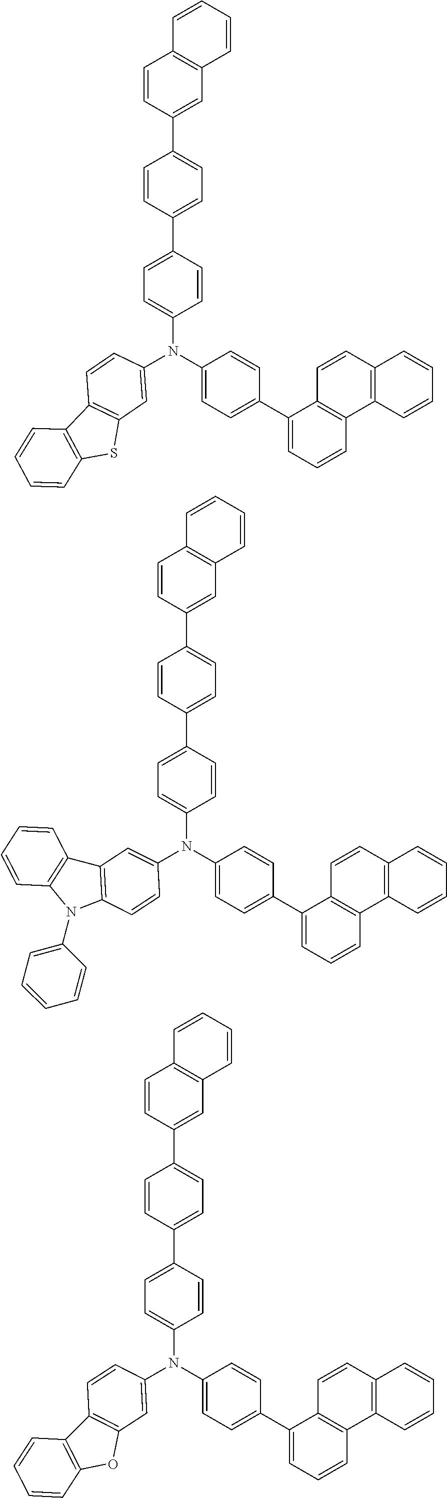 [SMILES: c1ccc2cc(-c3ccc(-c4ccc(N(c5ccc(-c6cccc7c6ccc6ccccc67)cc5)c5ccc6c(c5)sc5ccccc56)cc4)cc3)ccc2c1 ; c1ccc(-n2c3ccccc3c3cc(N(c4ccc(-c5ccc(-c6ccc7ccccc7c6)cc5)cc4)c4ccc(-c5cccc6c5ccc5ccccc56)cc4)ccc32)cc1 ; c1ccc2cc(-c3ccc(-c4ccc(N(c5ccc(-c6cccc7c6ccc6ccccc67)cc5)c5ccc6c(c5)oc5ccccc56)cc4)cc3)ccc2c1]

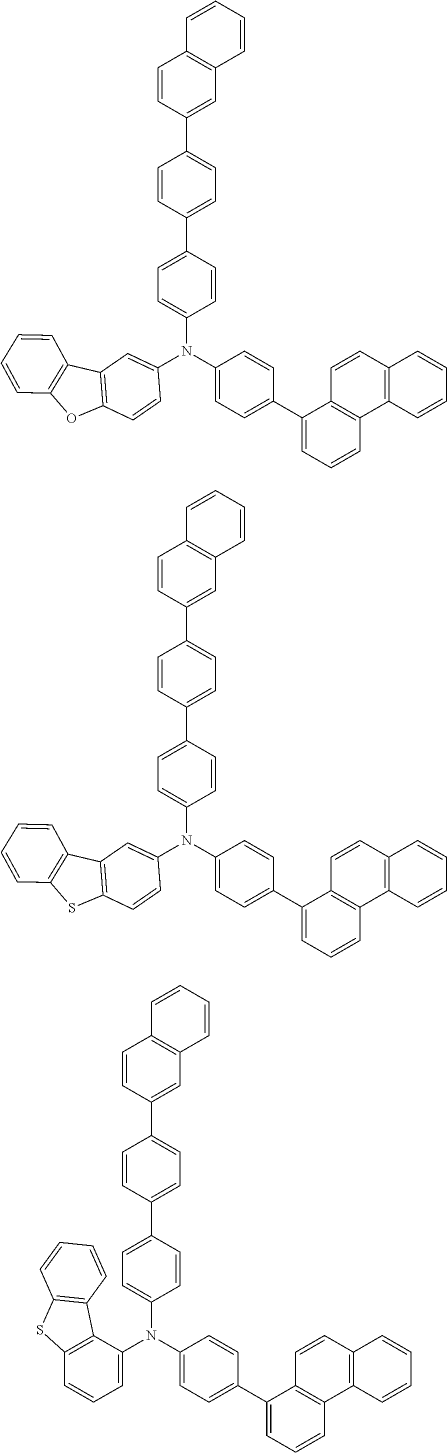 [SMILES: c1ccc2cc(-c3ccc(-c4ccc(N(c5ccc(-c6cccc7c6ccc6ccccc67)cc5)c5ccc6oc7ccccc7c6c5)cc4)cc3)ccc2c1 ; c1ccc2cc(-c3ccc(-c4ccc(N(c5ccc(-c6cccc7c6ccc6ccccc67)cc5)c5ccc6sc7ccccc7c6c5)cc4)cc3)ccc2c1 ; c1ccc2cc(-c3ccc(-c4ccc(N(c5ccc(-c6cccc7c6ccc6ccccc67)cc5)c5cccc6sc7ccccc7c56)cc4)cc3)ccc2c1]

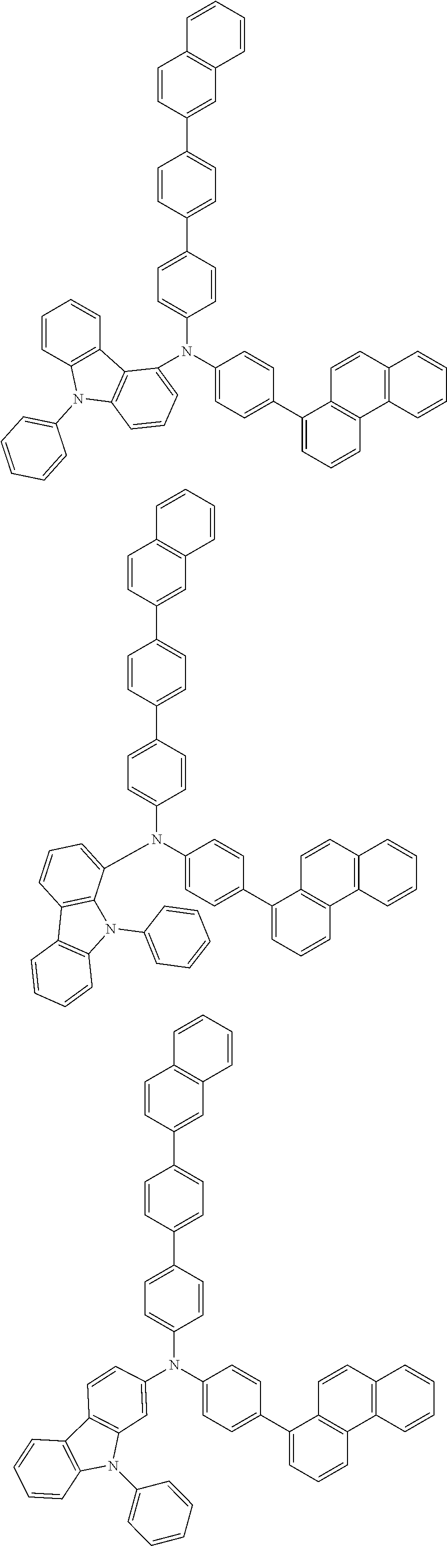 [SMILES: c1ccc(-n2c3ccccc3c3c(N(c4ccc(-c5ccc(-c6ccc7ccccc7c6)cc5)cc4)c4ccc(-c5cccc6c5ccc5ccccc56)cc4)cccc32)cc1 ; c1ccc(-n2c3ccccc3c3cccc(N(c4ccc(-c5ccc(-c6ccc7ccccc7c6)cc5)cc4)c4ccc(-c5cccc6c5ccc5ccccc56)cc4)c32)cc1 ; c1ccc(-n2c3ccccc3c3ccc(N(c4ccc(-c5ccc(-c6ccc7ccccc7c6)cc5)cc4)c4ccc(-c5cccc6c5ccc5ccccc56)cc4)cc32)cc1]

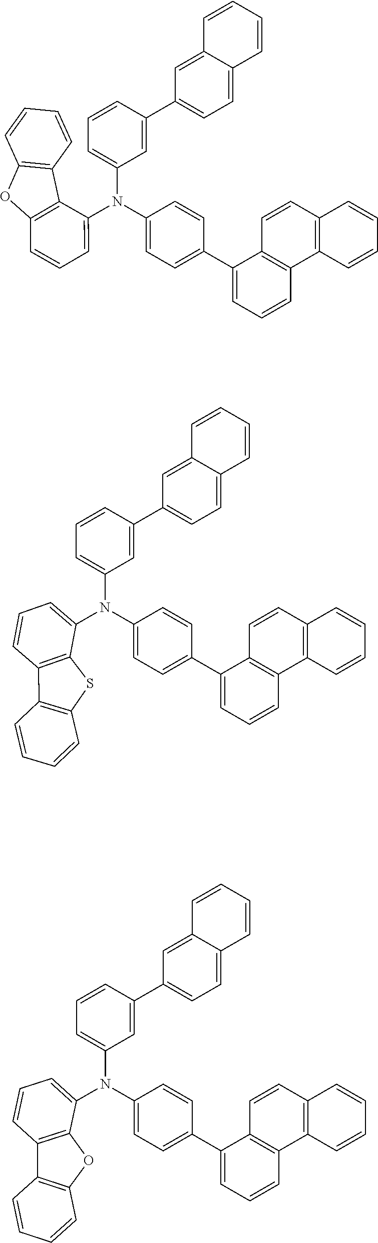 [SMILES: c1cc(-c2ccc3ccccc3c2)cc(N(c2ccc(-c3cccc4c3ccc3ccccc34)cc2)c2cccc3oc4ccccc4c23)c1 ; c1cc(-c2ccc3ccccc3c2)cc(N(c2ccc(-c3cccc4c3ccc3ccccc34)cc2)c2cccc3c2sc2ccccc23)c1 ; c1cc(-c2ccc3ccccc3c2)cc(N(c2ccc(-c3cccc4c3ccc3ccccc34)cc2)c2cccc3c2oc2ccccc23)c1]

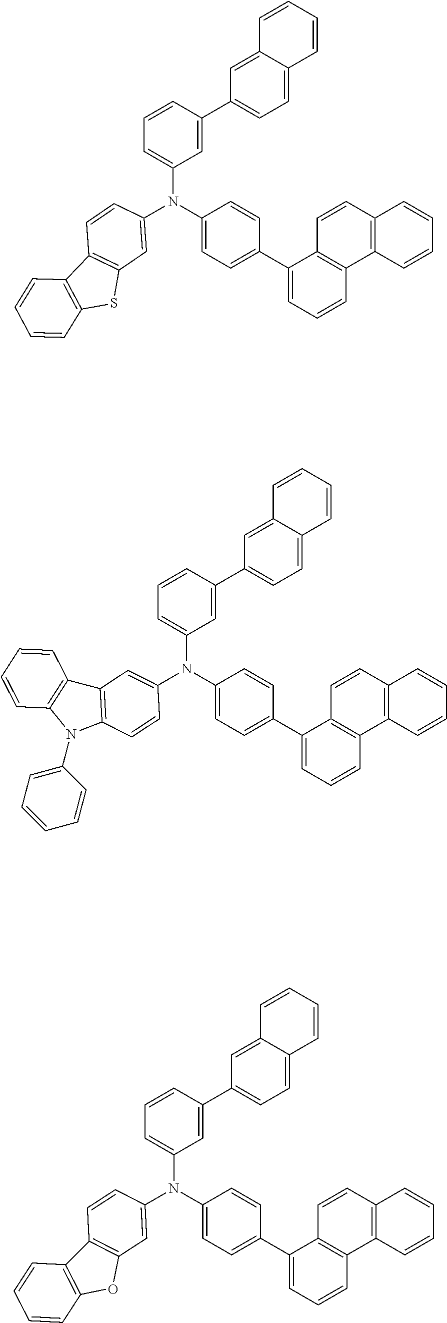 [SMILES: c1cc(-c2ccc3ccccc3c2)cc(N(c2ccc(-c3cccc4c3ccc3ccccc34)cc2)c2ccc3c(c2)sc2ccccc23)c1 ; c1ccc(-n2c3ccccc3c3cc(N(c4ccc(-c5cccc6c5ccc5ccccc56)cc4)c4cccc(-c5ccc6ccccc6c5)c4)ccc32)cc1 ; c1cc(-c2ccc3ccccc3c2)cc(N(c2ccc(-c3cccc4c3ccc3ccccc34)cc2)c2ccc3c(c2)oc2ccccc23)c1]

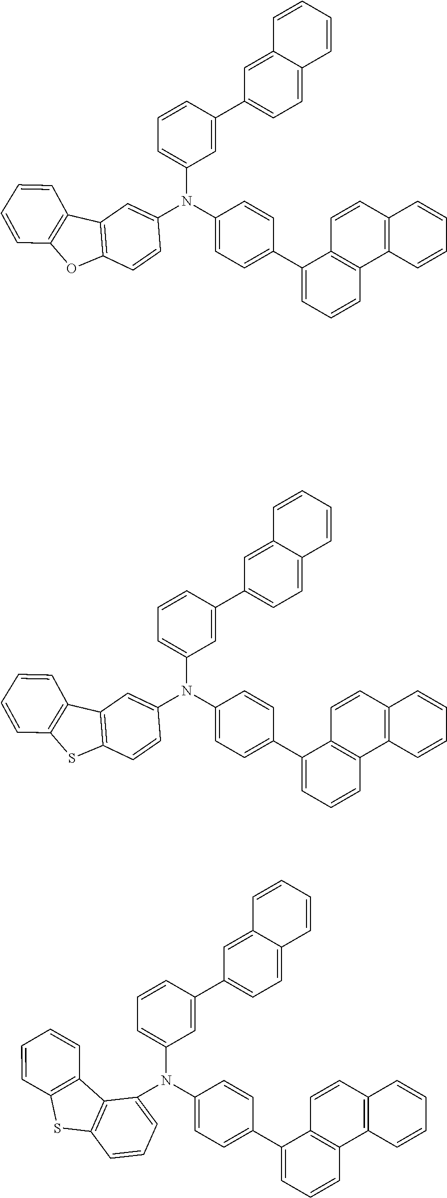 [SMILES: c1cc(-c2ccc3ccccc3c2)cc(N(c2ccc(-c3cccc4c3ccc3ccccc34)cc2)c2ccc3oc4ccccc4c3c2)c1 ; c1cc(-c2ccc3ccccc3c2)cc(N(c2ccc(-c3cccc4c3ccc3ccccc34)cc2)c2ccc3sc4ccccc4c3c2)c1 ; c1cc(-c2ccc3ccccc3c2)cc(N(c2ccc(-c3cccc4c3ccc3ccccc34)cc2)c2cccc3sc4ccccc4c23)c1]

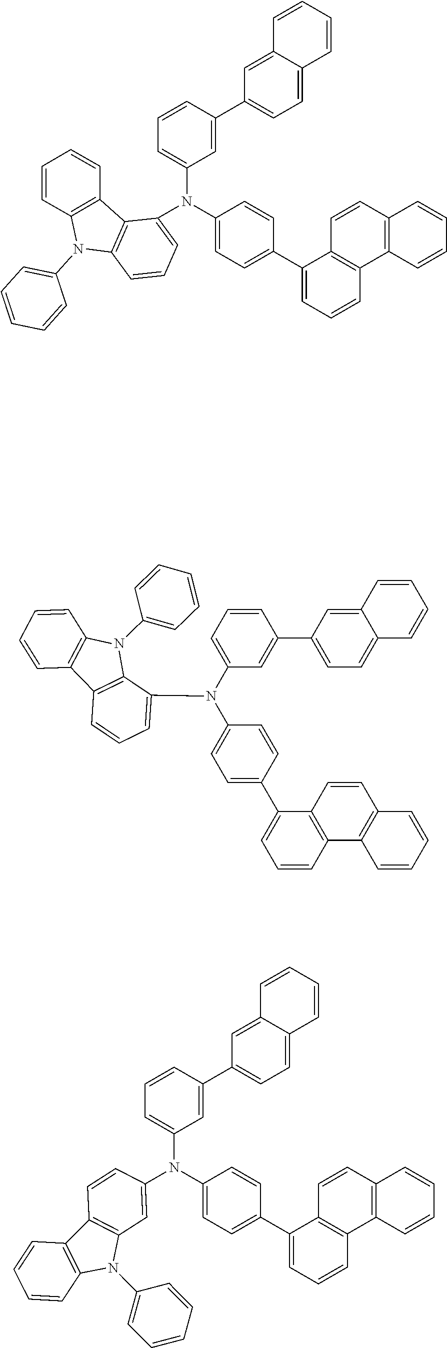 [SMILES: c1ccc(-n2c3ccccc3c3c(N(c4ccc(-c5cccc6c5ccc5ccccc56)cc4)c4cccc(-c5ccc6ccccc6c5)c4)cccc32)cc1 ; c1ccc(-n2c3ccccc3c3cccc(N(c4ccc(-c5cccc6c5ccc5ccccc56)cc4)c4cccc(-c5ccc6ccccc6c5)c4)c32)cc1 ; c1ccc(-n2c3ccccc3c3ccc(N(c4ccc(-c5cccc6c5ccc5ccccc56)cc4)c4cccc(-c5ccc6ccccc6c5)c4)cc32)cc1]

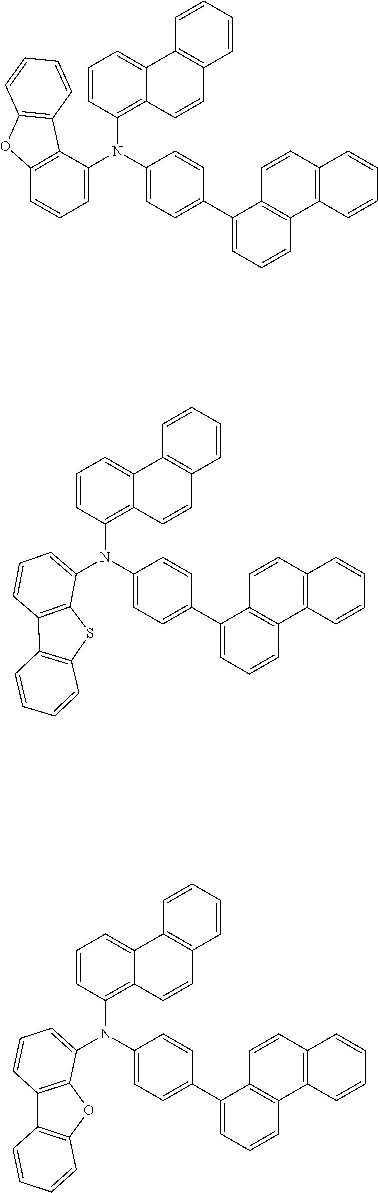 [SMILES: c1ccc2c(c1)ccc1c(-c3ccc(N(c4cccc5c4ccc4ccccc45)c4cccc5oc6ccccc6c45)cc3)cccc12 ; c1ccc2c(c1)ccc1c(-c3ccc(N(c4cccc5c4ccc4ccccc45)c4cccc5c4sc4ccccc45)cc3)cccc12 ; c1ccc2c(c1)ccc1c(-c3ccc(N(c4cccc5c4ccc4ccccc45)c4cccc5c4oc4ccccc45)cc3)cccc12]

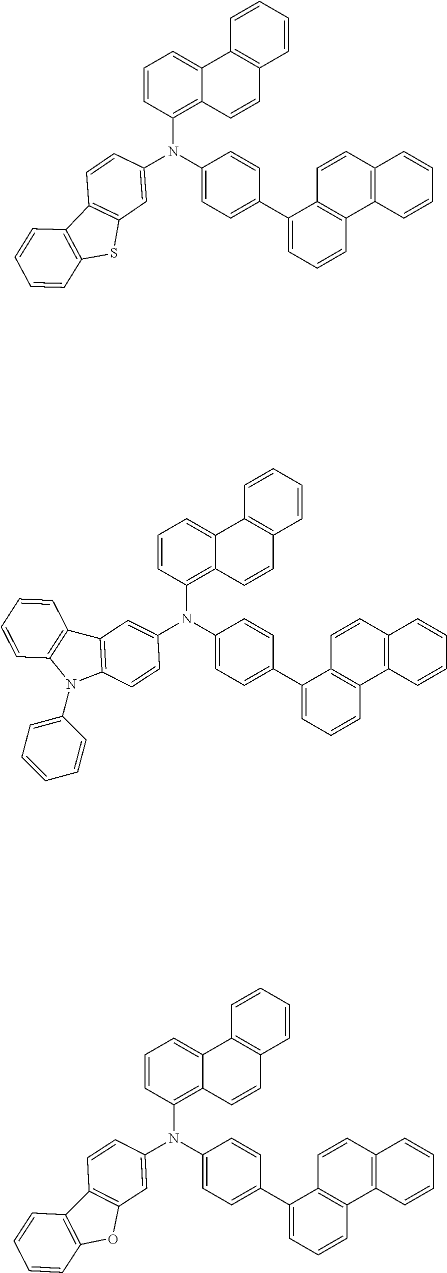 [SMILES: c1ccc2c(c1)ccc1c(-c3ccc(N(c4ccc5c(c4)sc4ccccc45)c4cccc5c4ccc4ccccc45)cc3)cccc12 ; c1ccc(-n2c3ccccc3c3cc(N(c4ccc(-c5cccc6c5ccc5ccccc56)cc4)c4cccc5c4ccc4ccccc45)ccc32)cc1 ; c1ccc2c(c1)ccc1c(-c3ccc(N(c4ccc5c(c4)oc4ccccc45)c4cccc5c4ccc4ccccc45)cc3)cccc12]

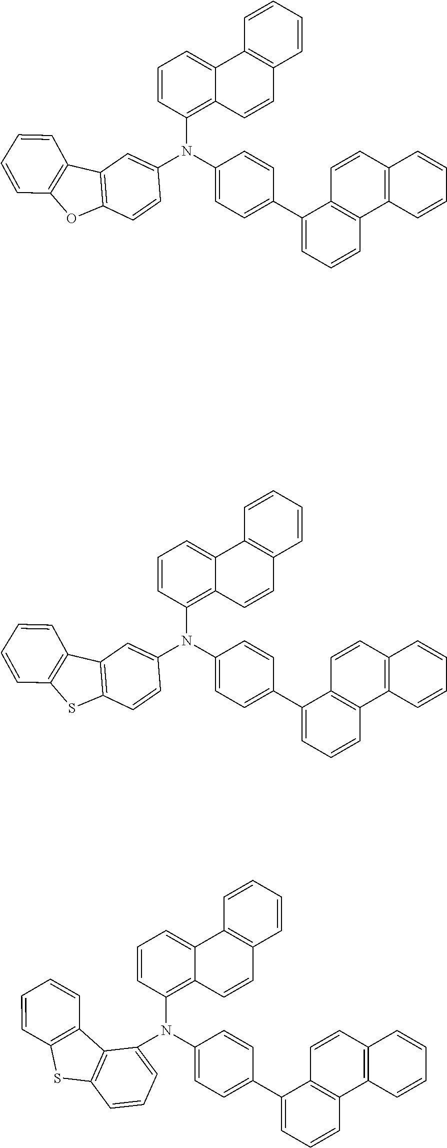 [SMILES: c1ccc2c(c1)ccc1c(-c3ccc(N(c4ccc5oc6ccccc6c5c4)c4cccc5c4ccc4ccccc45)cc3)cccc12 ; c1ccc2c(c1)ccc1c(-c3ccc(N(c4ccc5sc6ccccc6c5c4)c4cccc5c4ccc4ccccc45)cc3)cccc12 ; c1ccc2c(c1)ccc1c(-c3ccc(N(c4cccc5c4ccc4ccccc45)c4cccc5sc6ccccc6c45)cc3)cccc12]

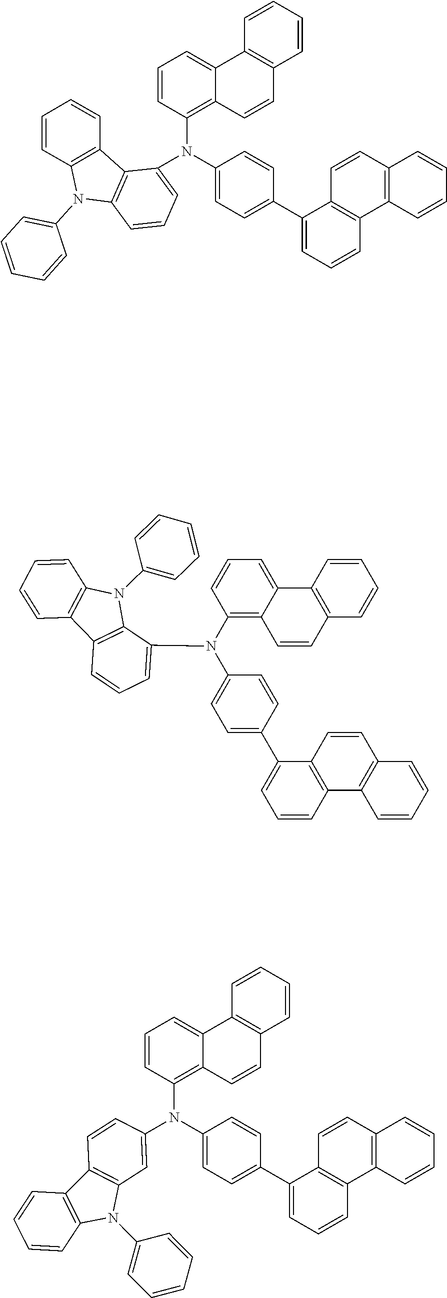 [SMILES: c1ccc(-n2c3ccccc3c3c(N(c4ccc(-c5cccc6c5ccc5ccccc56)cc4)c4cccc5c4ccc4ccccc45)cccc32)cc1 ; c1ccc(-n2c3ccccc3c3cccc(N(c4ccc(-c5cccc6c5ccc5ccccc56)cc4)c4cccc5c4ccc4ccccc45)c32)cc1 ; c1ccc(-n2c3ccccc3c3ccc(N(c4ccc(-c5cccc6c5ccc5ccccc56)cc4)c4cccc5c4ccc4ccccc45)cc32)cc1]

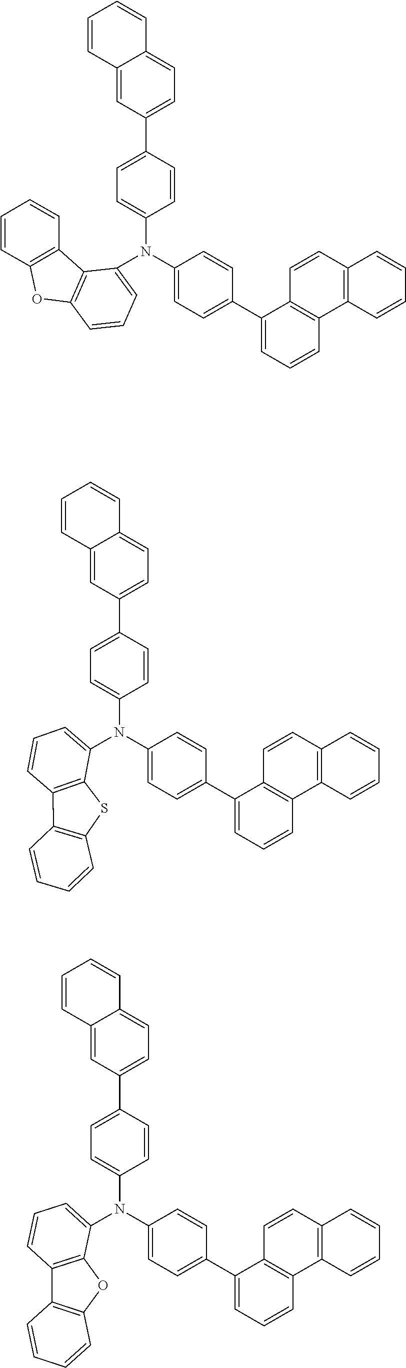 [SMILES: c1ccc2cc(-c3ccc(N(c4ccc(-c5cccc6c5ccc5ccccc56)cc4)c4cccc5oc6ccccc6c45)cc3)ccc2c1 ; c1ccc2cc(-c3ccc(N(c4ccc(-c5cccc6c5ccc5ccccc56)cc4)c4cccc5c4sc4ccccc45)cc3)ccc2c1 ; c1ccc2cc(-c3ccc(N(c4ccc(-c5cccc6c5ccc5ccccc56)cc4)c4cccc5c4oc4ccccc45)cc3)ccc2c1]

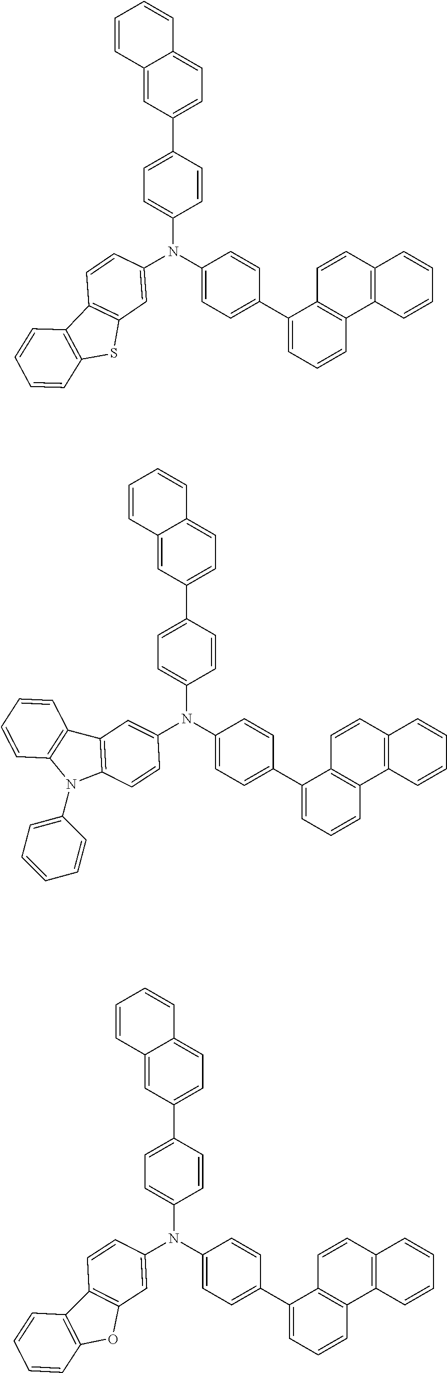 [SMILES: c1ccc2cc(-c3ccc(N(c4ccc(-c5cccc6c5ccc5ccccc56)cc4)c4ccc5c(c4)sc4ccccc45)cc3)ccc2c1 ; c1ccc(-n2c3ccccc3c3cc(N(c4ccc(-c5ccc6ccccc6c5)cc4)c4ccc(-c5cccc6c5ccc5ccccc56)cc4)ccc32)cc1 ; c1ccc2cc(-c3ccc(N(c4ccc(-c5cccc6c5ccc5ccccc56)cc4)c4ccc5c(c4)oc4ccccc45)cc3)ccc2c1]

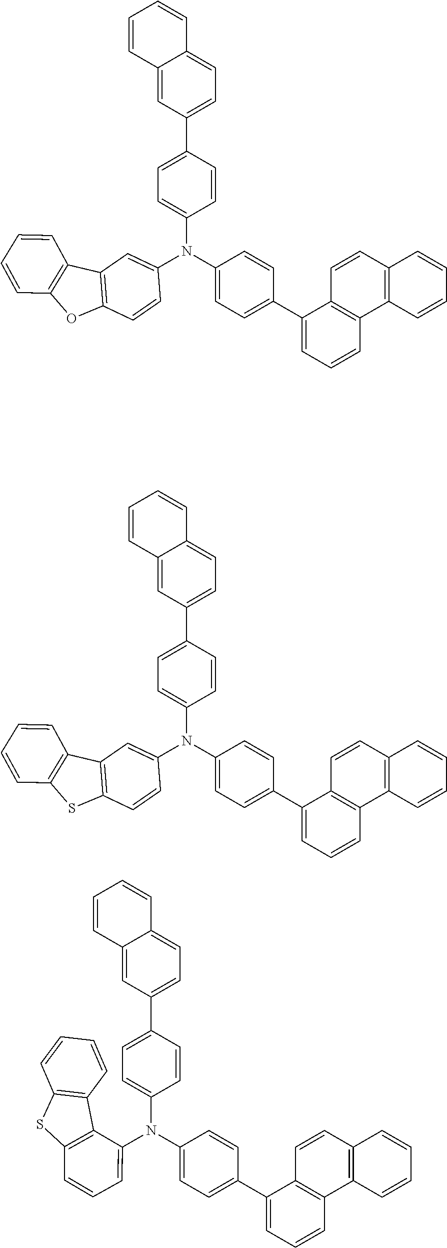 [SMILES: c1ccc2cc(-c3ccc(N(c4ccc(-c5cccc6c5ccc5ccccc56)cc4)c4ccc5oc6ccccc6c5c4)cc3)ccc2c1 ; c1ccc2cc(-c3ccc(N(c4ccc(-c5cccc6c5ccc5ccccc56)cc4)c4ccc5sc6ccccc6c5c4)cc3)ccc2c1 ; c1ccc2cc(-c3ccc(N(c4ccc(-c5cccc6c5ccc5ccccc56)cc4)c4cccc5sc6ccccc6c45)cc3)ccc2c1]

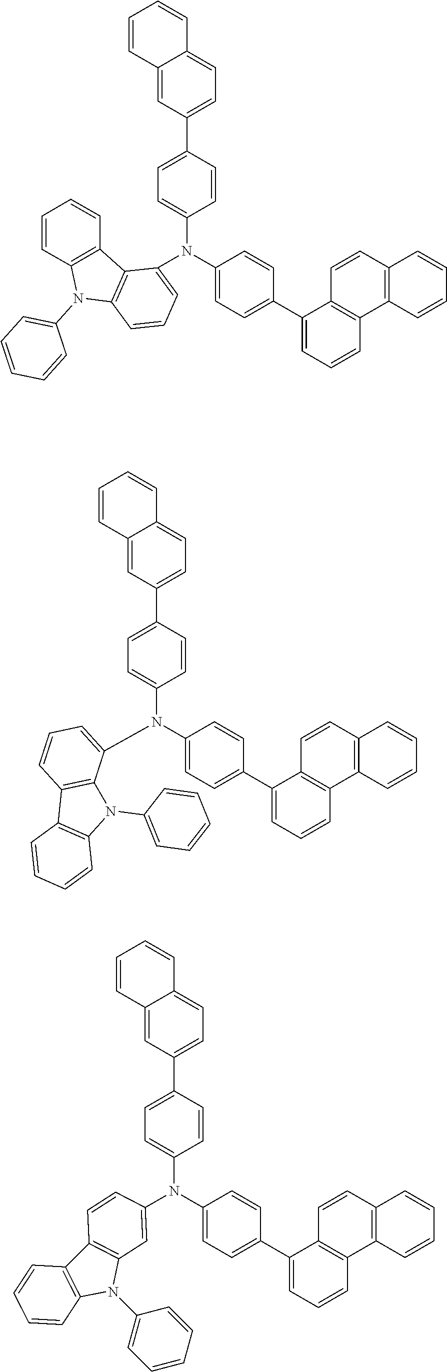 [SMILES: c1ccc(-n2c3ccccc3c3c(N(c4ccc(-c5ccc6ccccc6c5)cc4)c4ccc(-c5cccc6c5ccc5ccccc56)cc4)cccc32)cc1 ; c1ccc(-n2c3ccccc3c3cccc(N(c4ccc(-c5ccc6ccccc6c5)cc4)c4ccc(-c5cccc6c5ccc5ccccc56)cc4)c32)cc1 ; c1ccc(-n2c3ccccc3c3ccc(N(c4ccc(-c5ccc6ccccc6c5)cc4)c4ccc(-c5cccc6c5ccc5ccccc56)cc4)cc32)cc1]

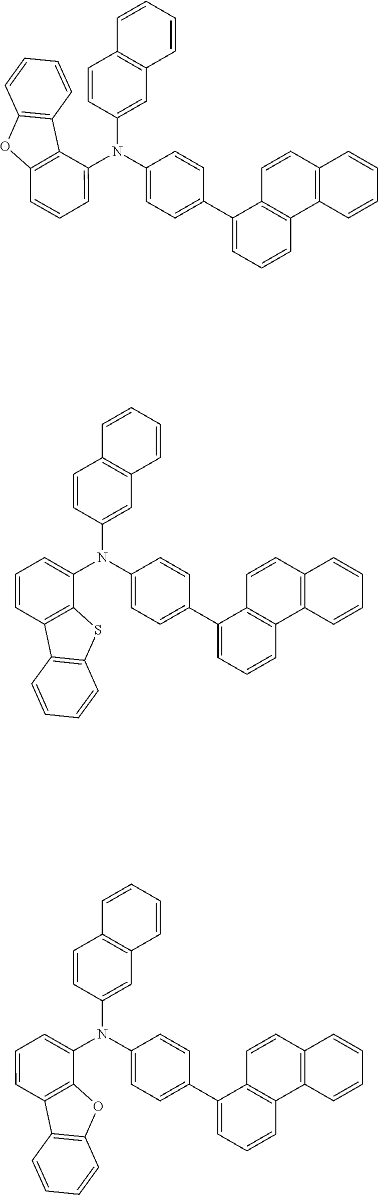 [SMILES: c1ccc2cc(N(c3ccc(-c4cccc5c4ccc4ccccc45)cc3)c3cccc4oc5ccccc5c34)ccc2c1 ; c1ccc2cc(N(c3ccc(-c4cccc5c4ccc4ccccc45)cc3)c3cccc4c3sc3ccccc34)ccc2c1 ; c1ccc2cc(N(c3ccc(-c4cccc5c4ccc4ccccc45)cc3)c3cccc4c3oc3ccccc34)ccc2c1]

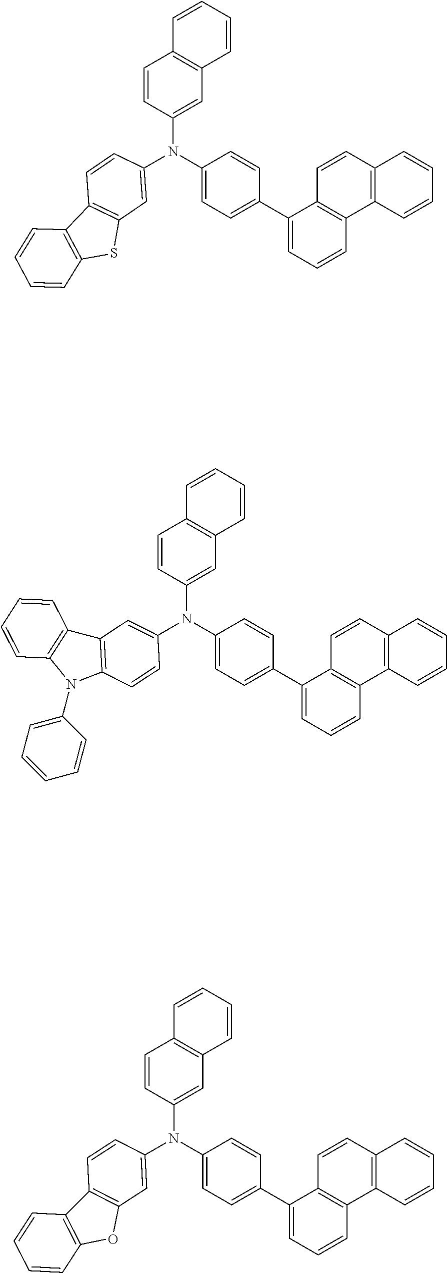 [SMILES: c1ccc2cc(N(c3ccc(-c4cccc5c4ccc4ccccc45)cc3)c3ccc4c(c3)sc3ccccc34)ccc2c1 ; c1ccc(-n2c3ccccc3c3cc(N(c4ccc(-c5cccc6c5ccc5ccccc56)cc4)c4ccc5ccccc5c4)ccc32)cc1 ; c1ccc2cc(N(c3ccc(-c4cccc5c4ccc4ccccc45)cc3)c3ccc4c(c3)oc3ccccc34)ccc2c1]

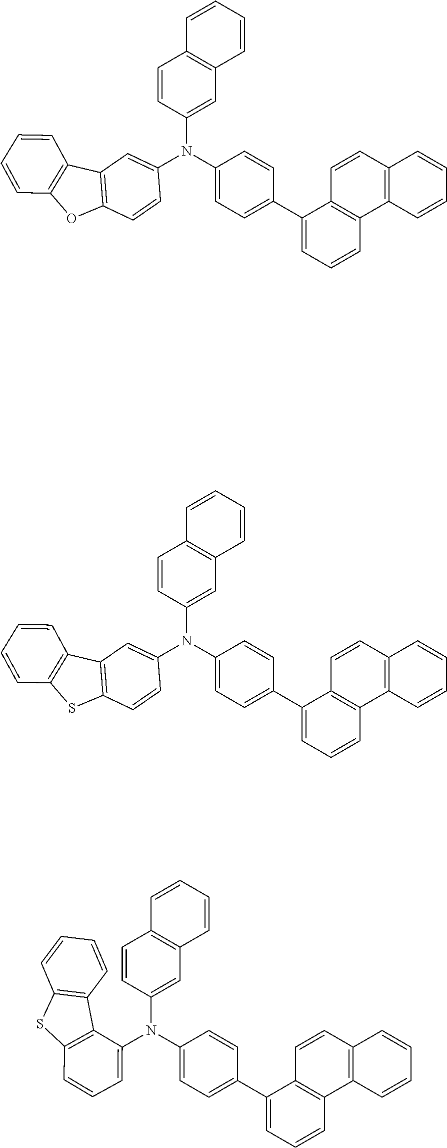 [SMILES: c1ccc2cc(N(c3ccc(-c4cccc5c4ccc4ccccc45)cc3)c3ccc4oc5ccccc5c4c3)ccc2c1 ; c1ccc2cc(N(c3ccc(-c4cccc5c4ccc4ccccc45)cc3)c3ccc4sc5ccccc5c4c3)ccc2c1 ; c1ccc2cc(N(c3ccc(-c4cccc5c4ccc4ccccc45)cc3)c3cccc4sc5ccccc5c34)ccc2c1]

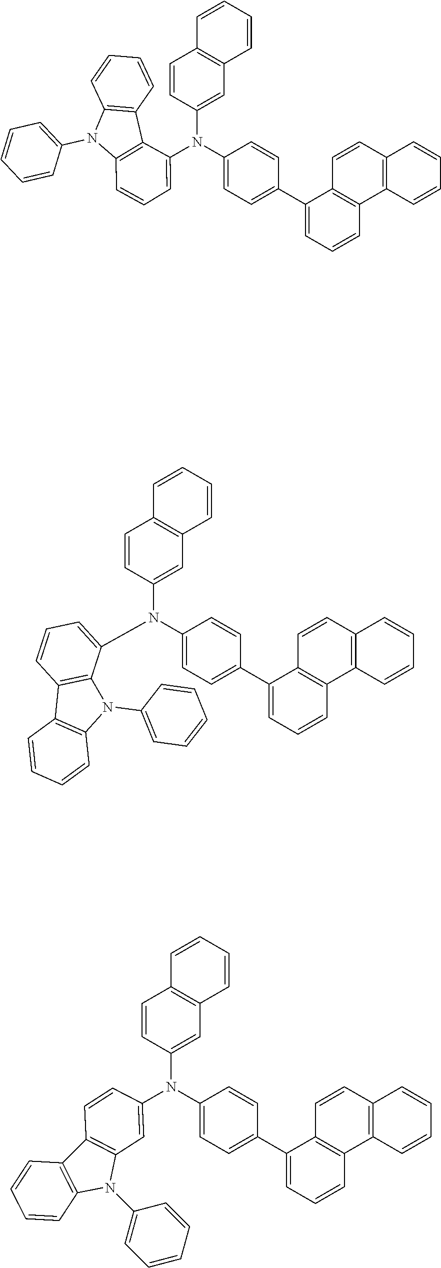 [SMILES: c1ccc(-n2c3ccccc3c3c(N(c4ccc(-c5cccc6c5ccc5ccccc56)cc4)c4ccc5ccccc5c4)cccc32)cc1 ; c1ccc(-n2c3ccccc3c3cccc(N(c4ccc(-c5cccc6c5ccc5ccccc56)cc4)c4ccc5ccccc5c4)c32)cc1 ; c1ccc(-n2c3ccccc3c3ccc(N(c4ccc(-c5cccc6c5ccc5ccccc56)cc4)c4ccc5ccccc5c4)cc32)cc1]

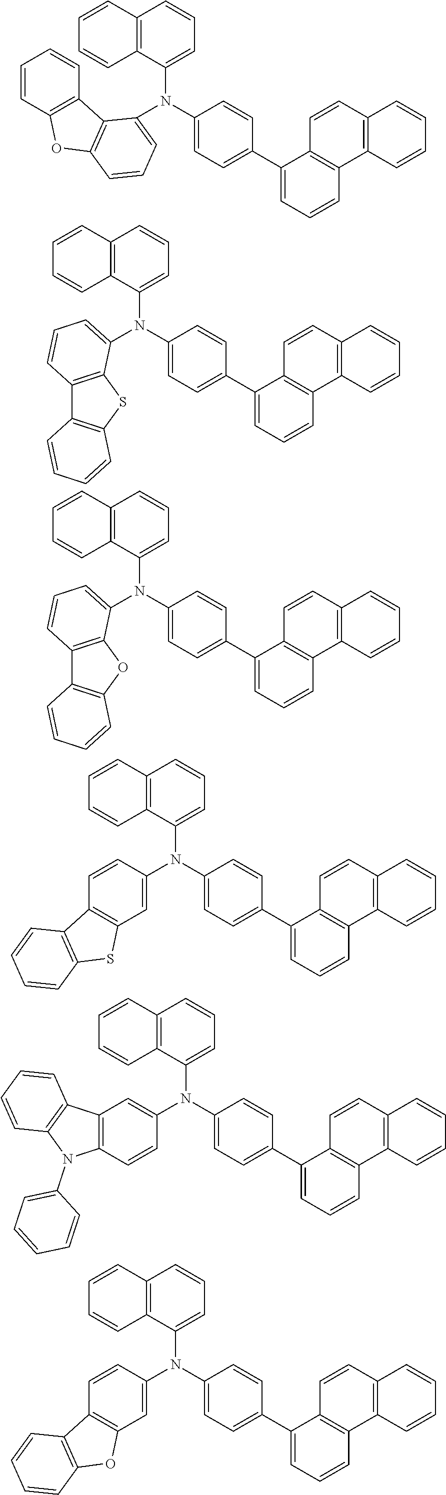

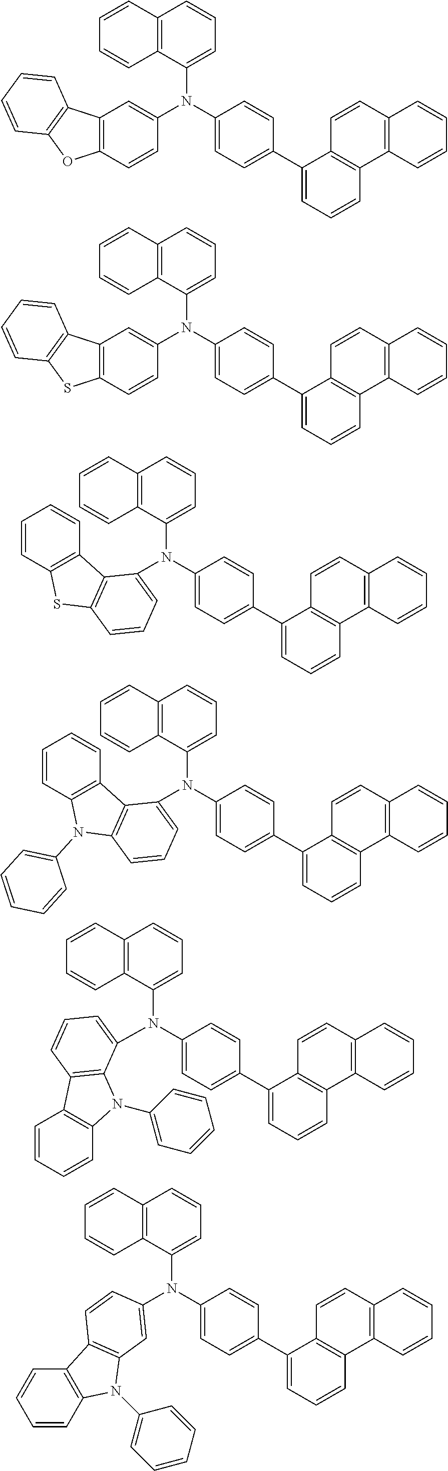

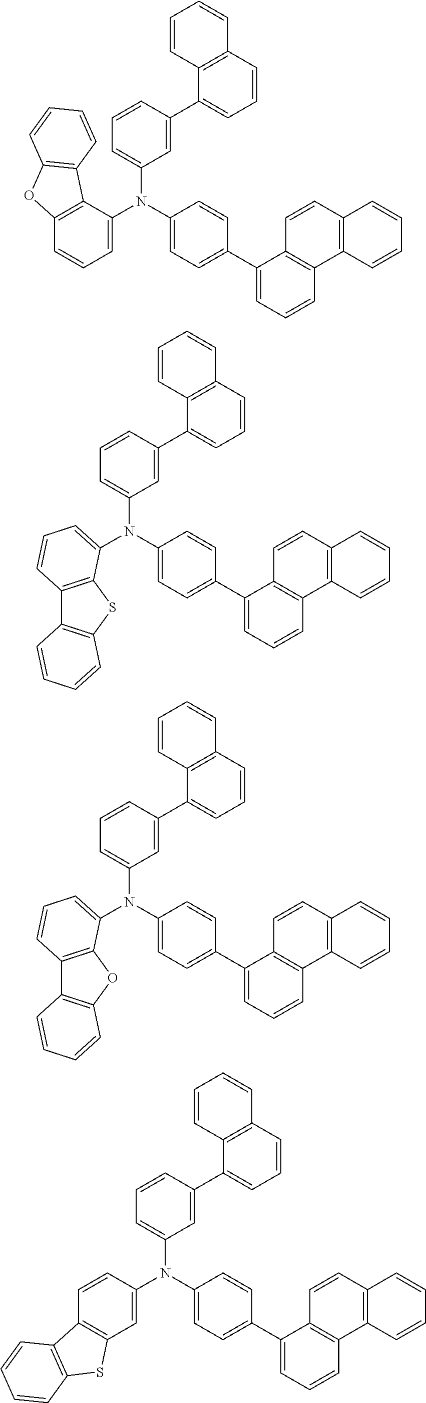 [SMILES: c1cc(-c2cccc3ccccc23)cc(N(c2ccc(-c3cccc4c3ccc3ccccc34)cc2)c2cccc3oc4ccccc4c23)c1 ; c1cc(-c2cccc3ccccc23)cc(N(c2ccc(-c3cccc4c3ccc3ccccc34)cc2)c2cccc3c2sc2ccccc23)c1 ; c1cc(-c2cccc3ccccc23)cc(N(c2ccc(-c3cccc4c3ccc3ccccc34)cc2)c2cccc3c2oc2ccccc23)c1 ; c1cc(-c2cccc3ccccc23)cc(N(c2ccc(-c3cccc4c3ccc3ccccc34)cc2)c2ccc3c(c2)sc2ccccc23)c1]

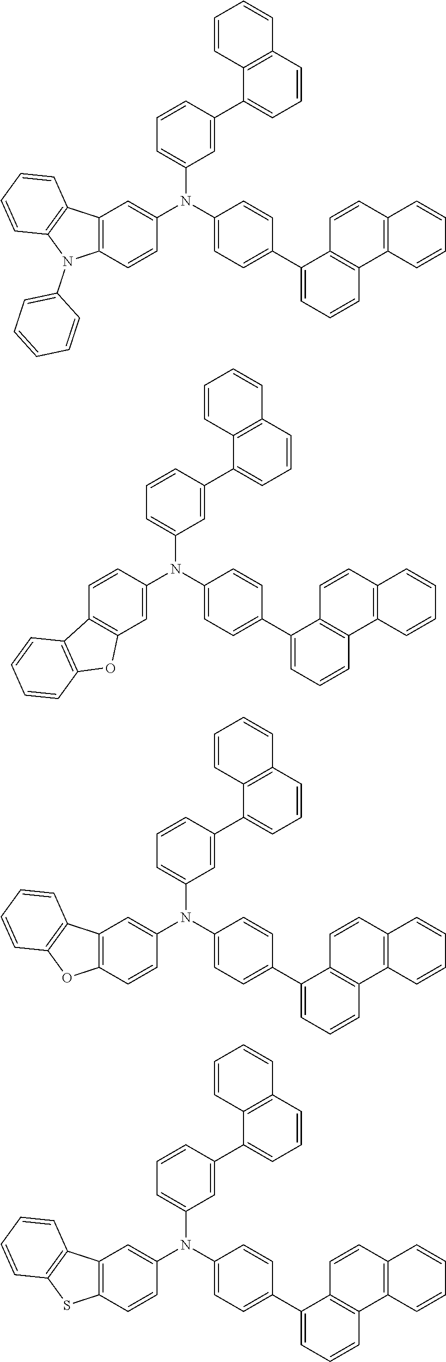 [SMILES: c1ccc(-n2c3ccccc3c3cc(N(c4ccc(-c5cccc6c5ccc5ccccc56)cc4)c4cccc(-c5cccc6ccccc56)c4)ccc32)cc1 ; c1cc(-c2cccc3ccccc23)cc(N(c2ccc(-c3cccc4c3ccc3ccccc34)cc2)c2ccc3c(c2)oc2ccccc23)c1 ; c1cc(-c2cccc3ccccc23)cc(N(c2ccc(-c3cccc4c3ccc3ccccc34)cc2)c2ccc3oc4ccccc4c3c2)c1 ; c1cc(-c2cccc3ccccc23)cc(N(c2ccc(-c3cccc4c3ccc3ccccc34)cc2)c2ccc3sc4ccccc4c3c2)c1]

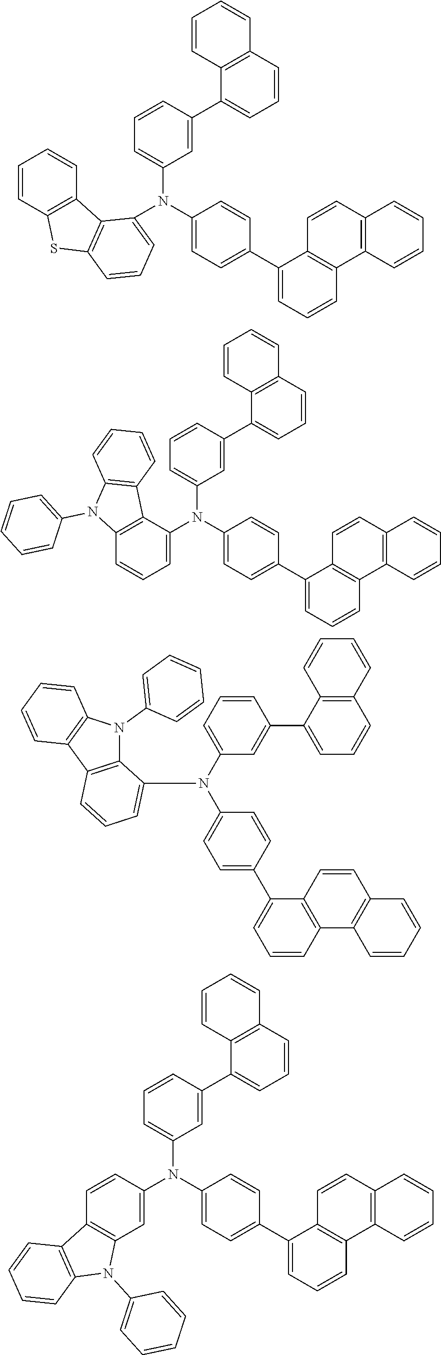

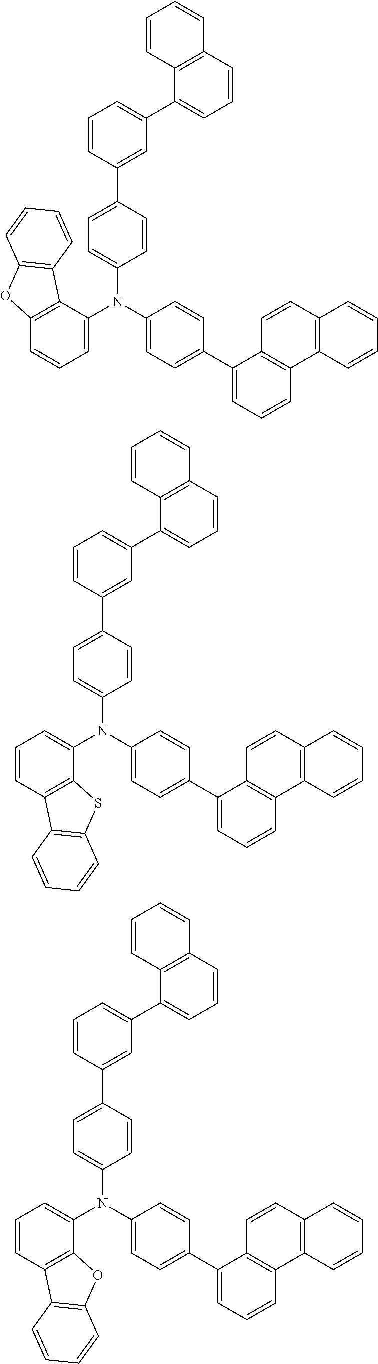 [SMILES: c1cc(-c2ccc(N(c3ccc(-c4cccc5c4ccc4ccccc45)cc3)c3cccc4oc5ccccc5c34)cc2)cc(-c2cccc3ccccc23)c1 ; c1cc(-c2ccc(N(c3ccc(-c4cccc5c4ccc4ccccc45)cc3)c3cccc4c3sc3ccccc34)cc2)cc(-c2cccc3ccccc23)c1 ; c1cc(-c2ccc(N(c3ccc(-c4cccc5c4ccc4ccccc45)cc3)c3cccc4c3oc3ccccc34)cc2)cc(-c2cccc3ccccc23)c1]

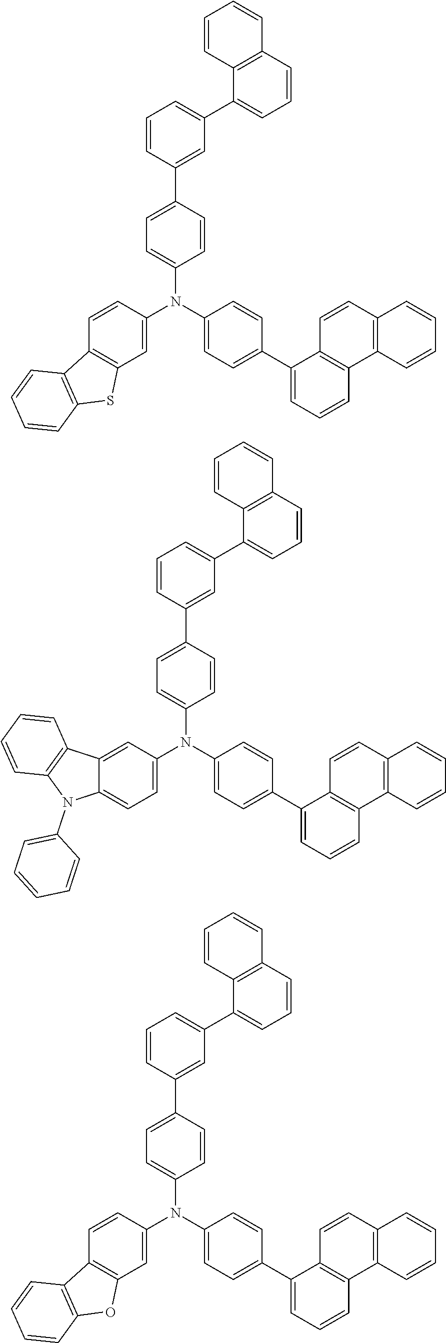 [SMILES: c1cc(-c2ccc(N(c3ccc(-c4cccc5c4ccc4ccccc45)cc3)c3ccc4c(c3)sc3ccccc34)cc2)cc(-c2cccc3ccccc23)c1 ; c1ccc(-n2c3ccccc3c3cc(N(c4ccc(-c5cccc(-c6cccc7ccccc67)c5)cc4)c4ccc(-c5cccc6c5ccc5ccccc56)cc4)ccc32)cc1 ; c1cc(-c2ccc(N(c3ccc(-c4cccc5c4ccc4ccccc45)cc3)c3ccc4c(c3)oc3ccccc34)cc2)cc(-c2cccc3ccccc23)c1]

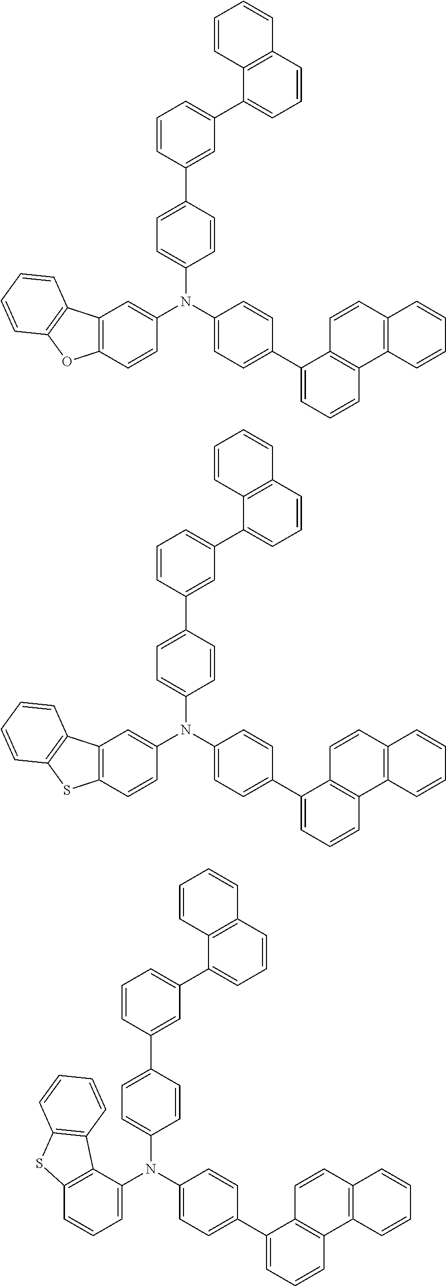 [SMILES: c1cc(-c2ccc(N(c3ccc(-c4cccc5c4ccc4ccccc45)cc3)c3ccc4oc5ccccc5c4c3)cc2)cc(-c2cccc3ccccc23)c1 ; c1cc(-c2ccc(N(c3ccc(-c4cccc5c4ccc4ccccc45)cc3)c3ccc4sc5ccccc5c4c3)cc2)cc(-c2cccc3ccccc23)c1 ; c1cc(-c2ccc(N(c3ccc(-c4cccc5c4ccc4ccccc45)cc3)c3cccc4sc5ccccc5c34)cc2)cc(-c2cccc3ccccc23)c1]

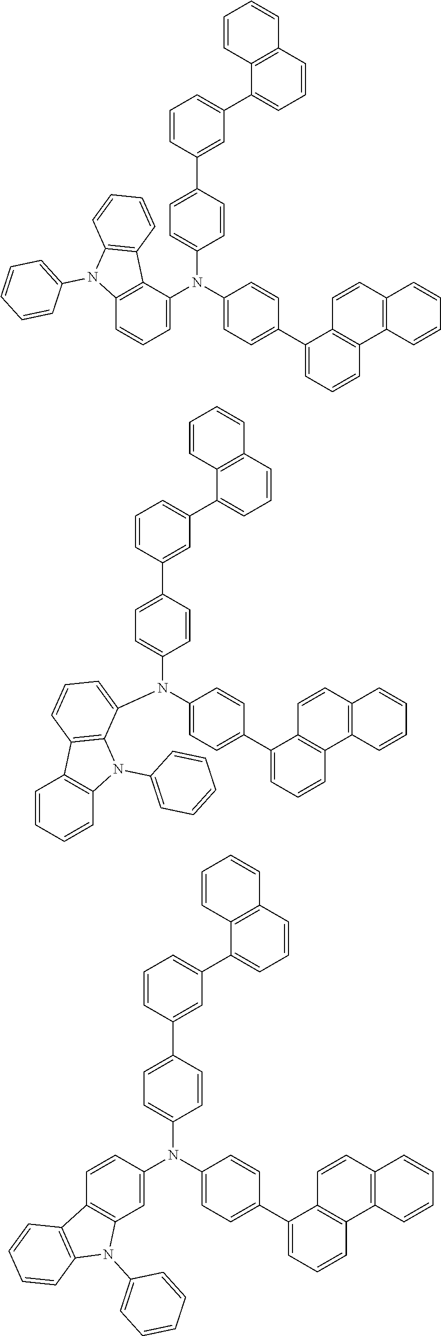 [SMILES: c1ccc(-n2c3ccccc3c3c(N(c4ccc(-c5cccc(-c6cccc7ccccc67)c5)cc4)c4ccc(-c5cccc6c5ccc5ccccc56)cc4)cccc32)cc1 ; c1ccc(-n2c3ccccc3c3cccc(N(c4ccc(-c5cccc(-c6cccc7ccccc67)c5)cc4)c4ccc(-c5cccc6c5ccc5ccccc56)cc4)c32)cc1 ; c1ccc(-n2c3ccccc3c3ccc(N(c4ccc(-c5cccc(-c6cccc7ccccc67)c5)cc4)c4ccc(-c5cccc6c5ccc5ccccc56)cc4)cc32)cc1]

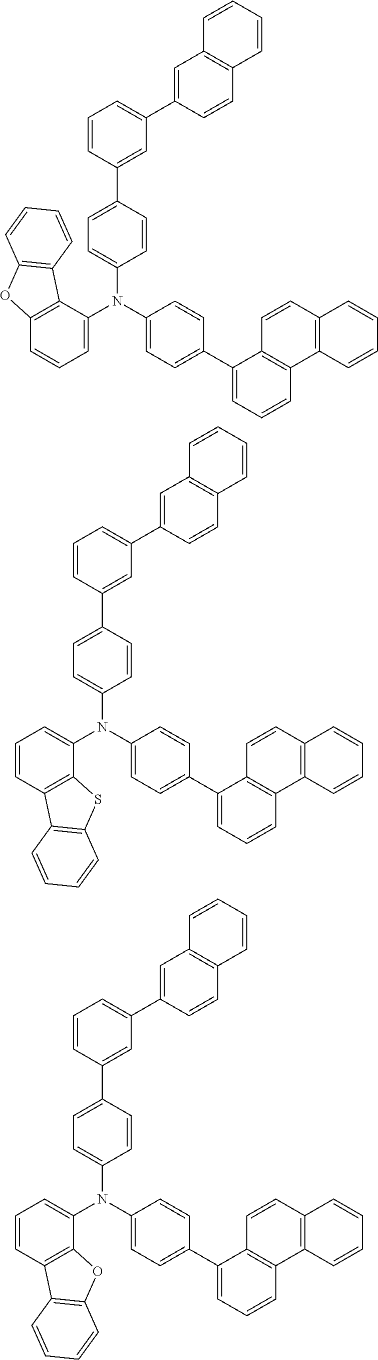

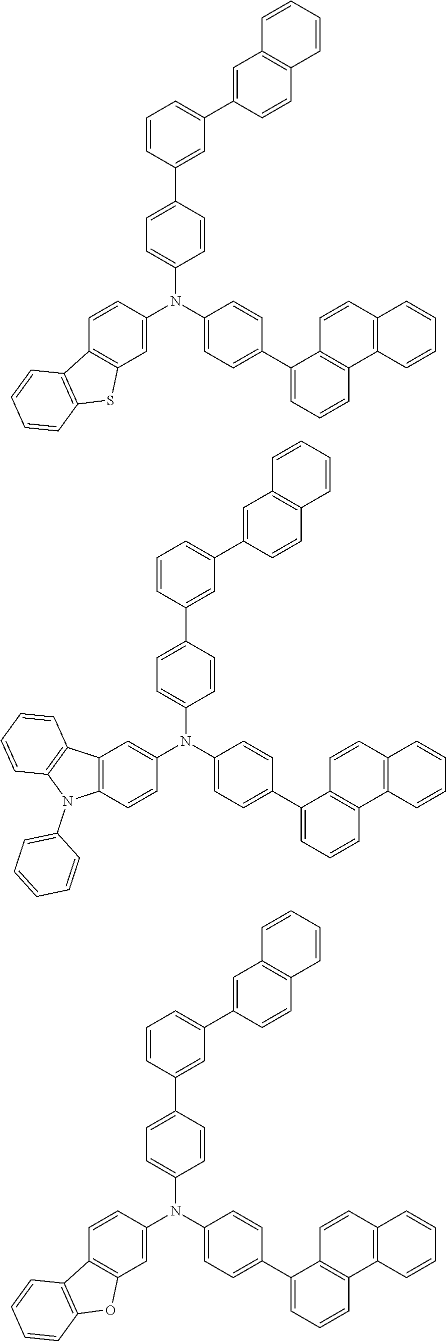

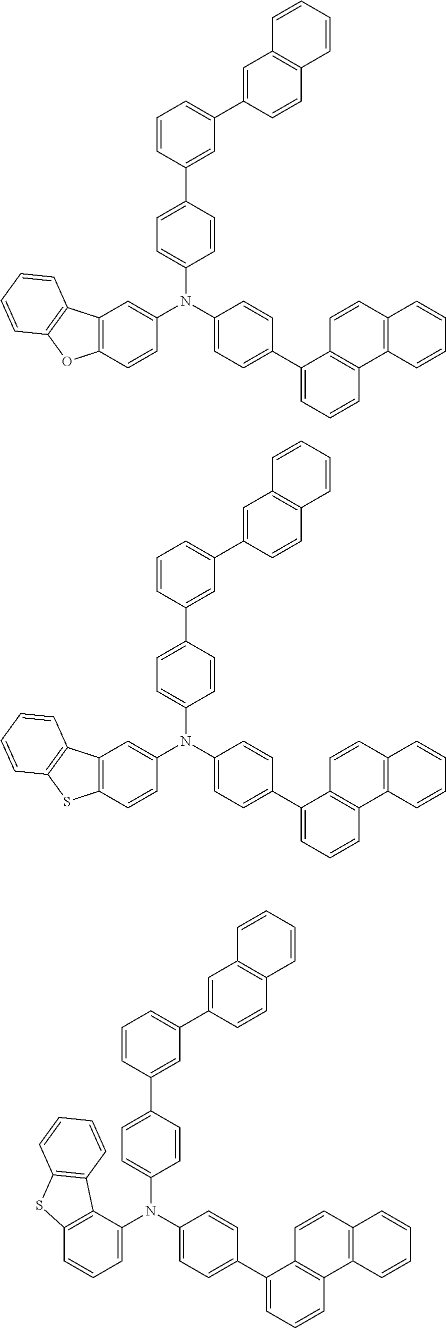

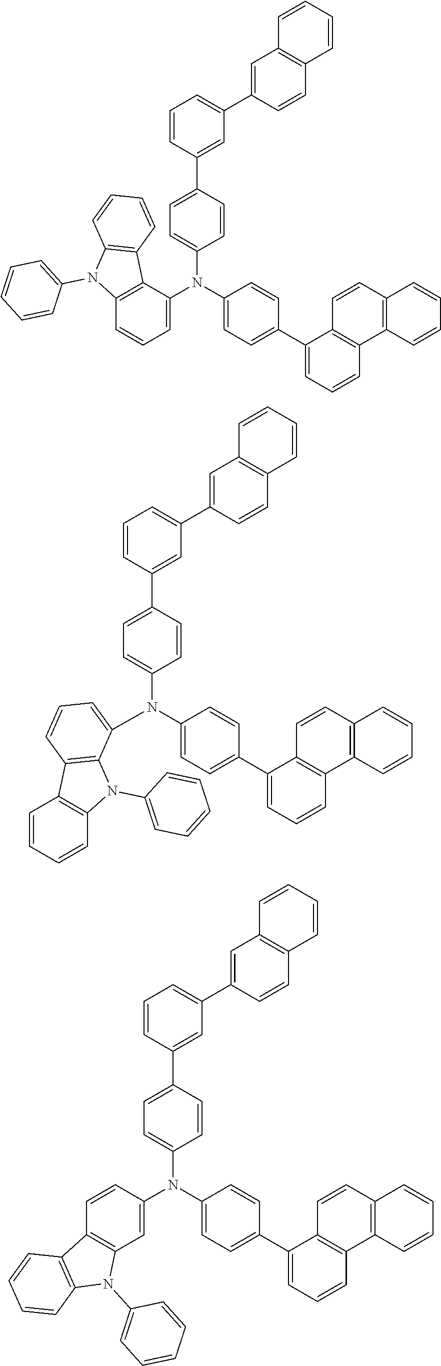 [SMILES: c1ccc(-n2c3ccccc3c3c(N(c4ccc(-c5cccc(-c6ccc7ccccc7c6)c5)cc4)c4ccc(-c5cccc6c5ccc5ccccc56)cc4)cccc32)cc1 ; c1ccc(-n2c3ccccc3c3cccc(N(c4ccc(-c5cccc(-c6ccc7ccccc7c6)c5)cc4)c4ccc(-c5cccc6c5ccc5ccccc56)cc4)c32)cc1 ; c1ccc(-n2c3ccccc3c3ccc(N(c4ccc(-c5cccc(-c6ccc7ccccc7c6)c5)cc4)c4ccc(-c5cccc6c5ccc5ccccc56)cc4)cc32)cc1]

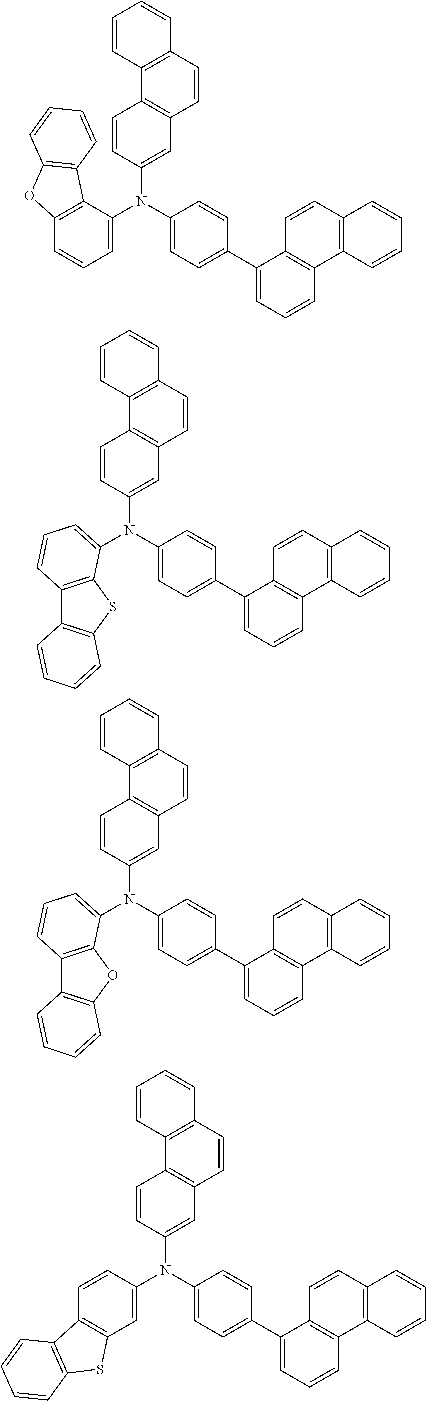

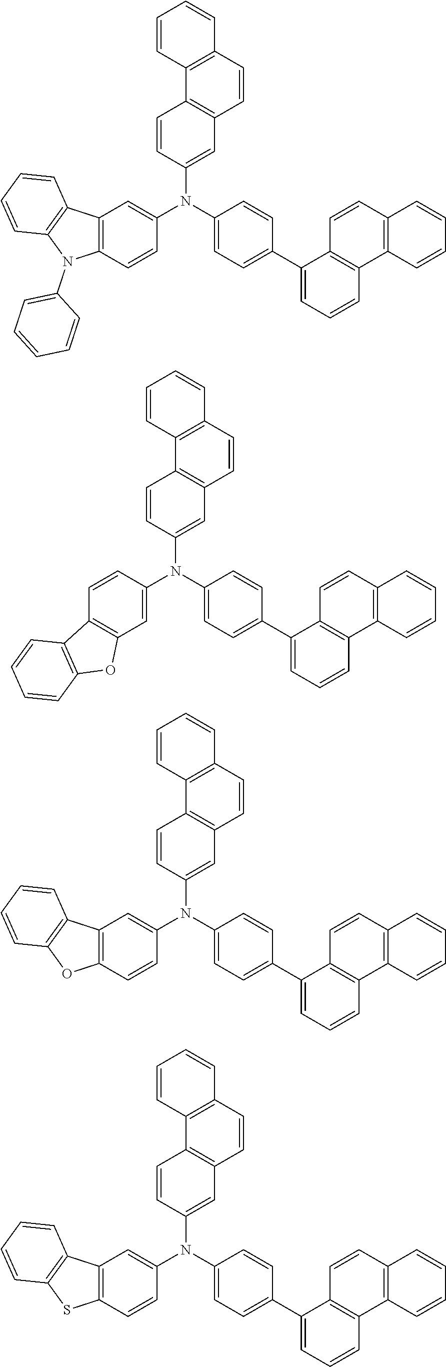 [SMILES: c1ccc(-n2c3ccccc3c3cc(N(c4ccc(-c5cccc6c5ccc5ccccc56)cc4)c4ccc5c(ccc6ccccc65)c4)ccc32)cc1 ; c1ccc2c(c1)ccc1cc(N(c3ccc(-c4cccc5c4ccc4ccccc45)cc3)c3ccc4c(c3)oc3ccccc34)ccc12 ; c1ccc2c(c1)ccc1cc(N(c3ccc(-c4cccc5c4ccc4ccccc45)cc3)c3ccc4oc5ccccc5c4c3)ccc12 ; c1ccc2c(c1)ccc1cc(N(c3ccc(-c4cccc5c4ccc4ccccc45)cc3)c3ccc4sc5ccccc5c4c3)ccc12]

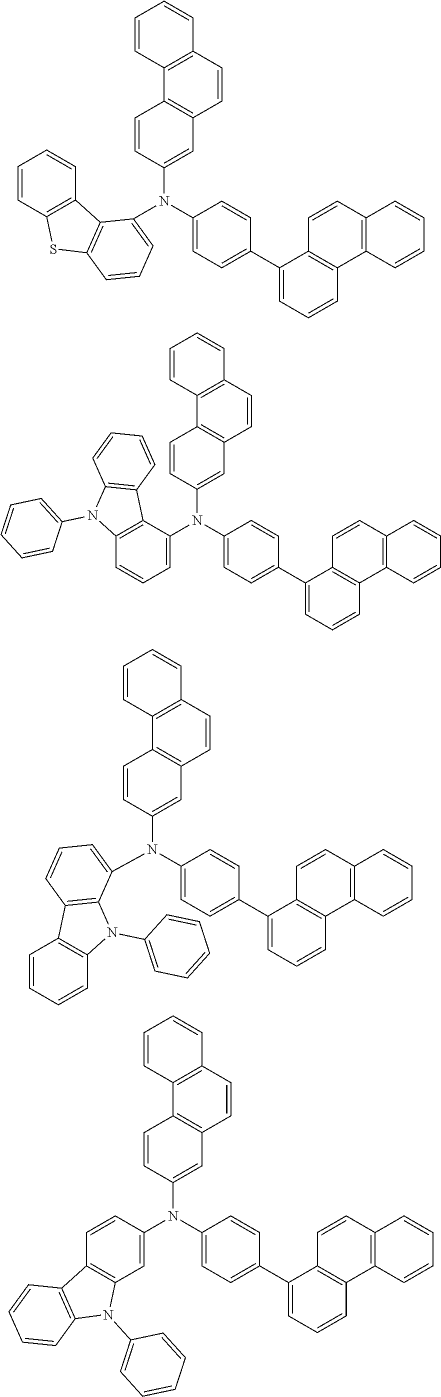

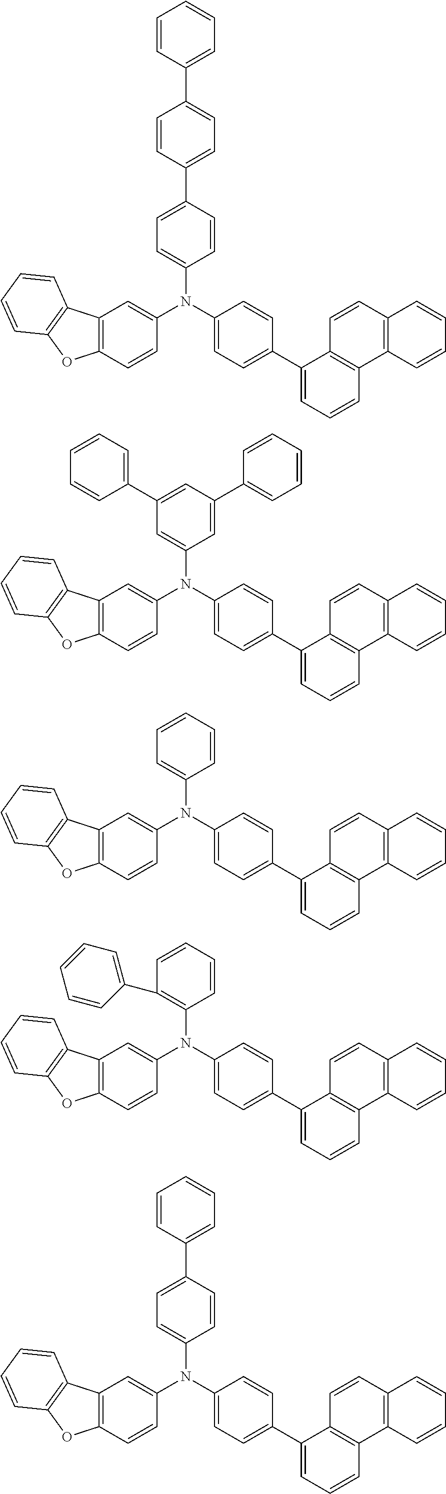

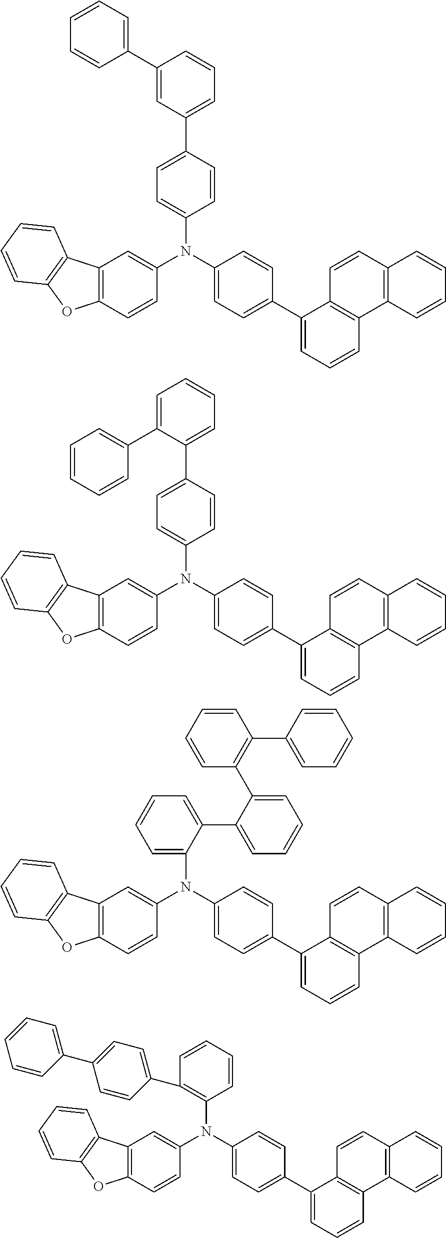

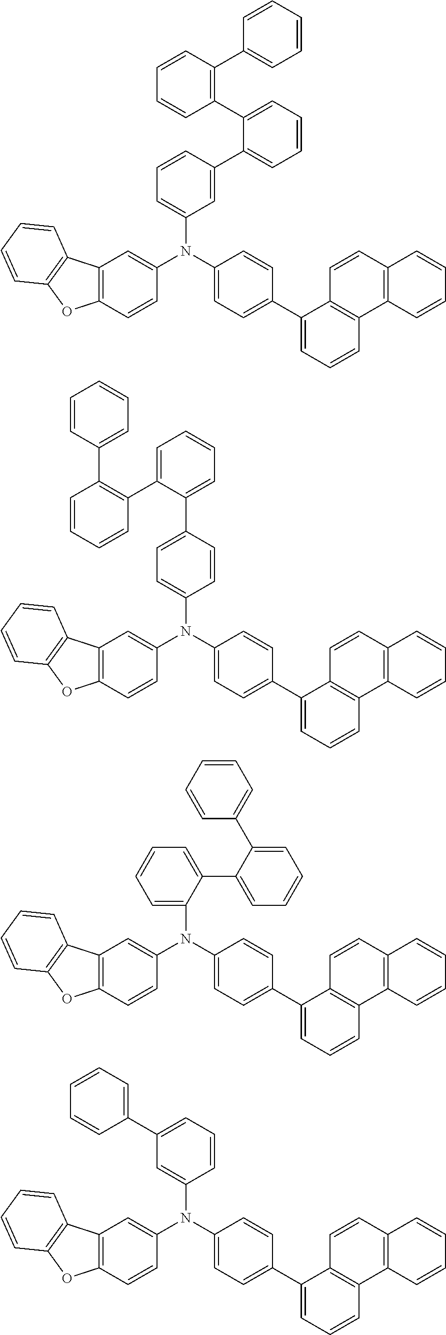 [SMILES: c1ccc(-c2ccccc2-c2ccccc2-c2cccc(N(c3ccc(-c4cccc5c4ccc4ccccc45)cc3)c3ccc4oc5ccccc5c4c3)c2)cc1 ; c1ccc(-c2ccccc2-c2ccccc2-c2ccc(N(c3ccc(-c4cccc5c4ccc4ccccc45)cc3)c3ccc4oc5ccccc5c4c3)cc2)cc1 ; c1ccc(-c2ccccc2-c2ccccc2N(c2ccc(-c3cccc4c3ccc3ccccc34)cc2)c2ccc3oc4ccccc4c3c2)cc1 ; c1ccc(-c2cccc(N(c3ccc(-c4cccc5c4ccc4ccccc45)cc3)c3ccc4oc5ccccc5c4c3)c2)cc1]

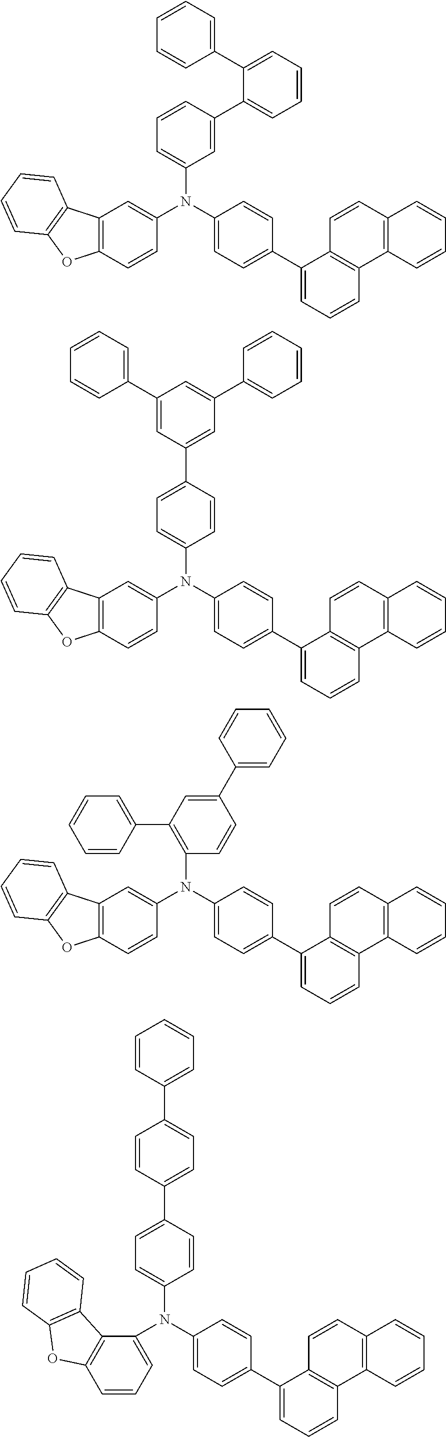

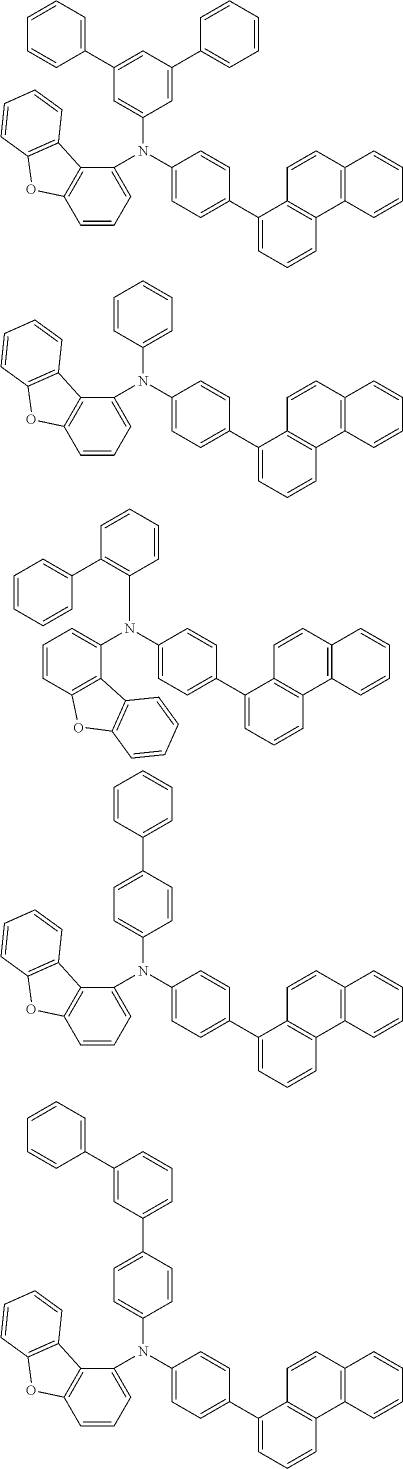

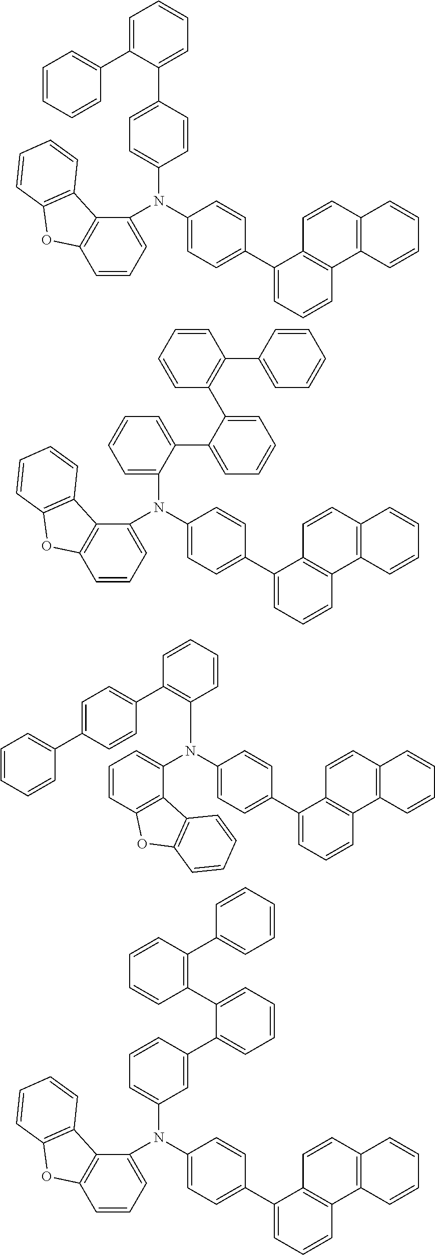

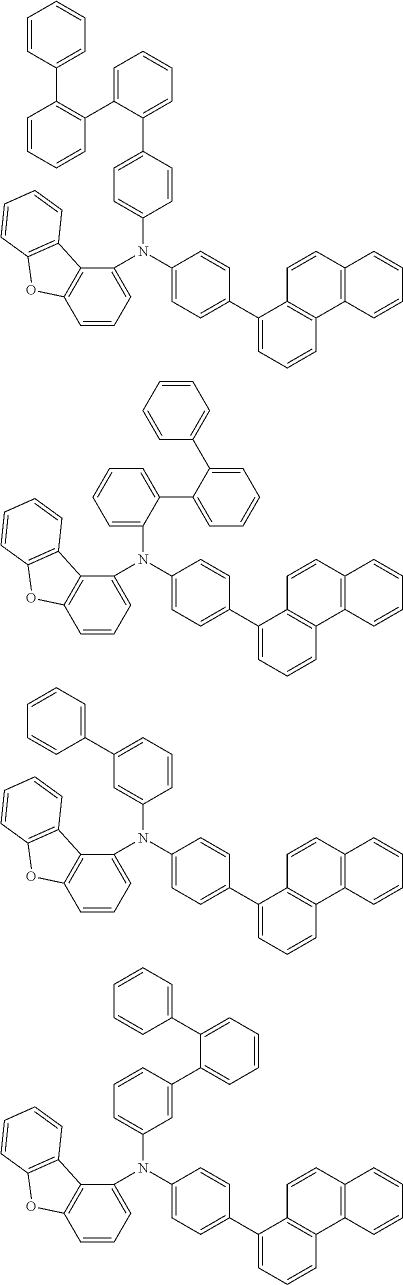 [SMILES: c1ccc(-c2ccccc2-c2ccccc2-c2ccc(N(c3ccc(-c4cccc5c4ccc4ccccc45)cc3)c3cccc4oc5ccccc5c34)cc2)cc1 ; c1ccc(-c2ccccc2-c2ccccc2N(c2ccc(-c3cccc4c3ccc3ccccc34)cc2)c2cccc3oc4ccccc4c23)cc1 ; c1ccc(-c2cccc(N(c3ccc(-c4cccc5c4ccc4ccccc45)cc3)c3cccc4oc5ccccc5c34)c2)cc1 ; c1ccc(-c2ccccc2-c2cccc(N(c3ccc(-c4cccc5c4ccc4ccccc45)cc3)c3cccc4oc5ccccc5c34)c2)cc1]

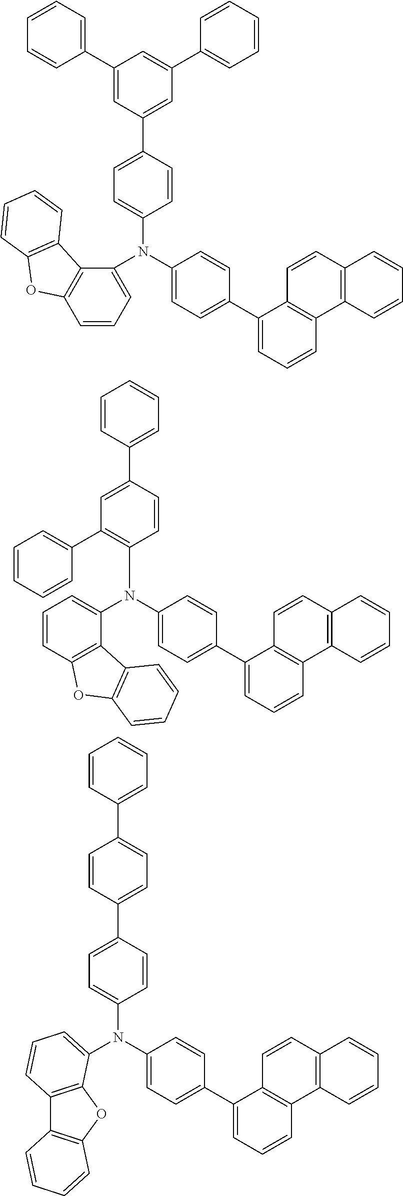

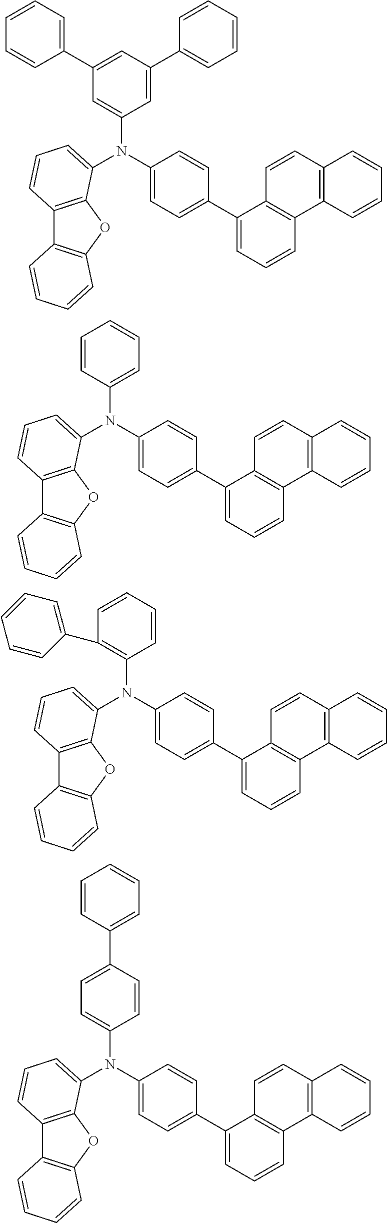

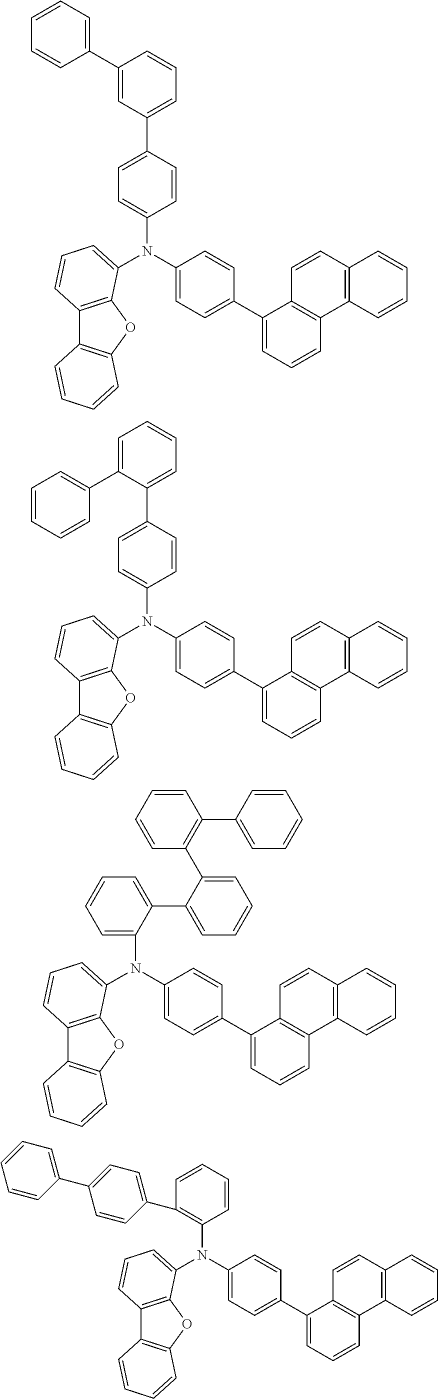

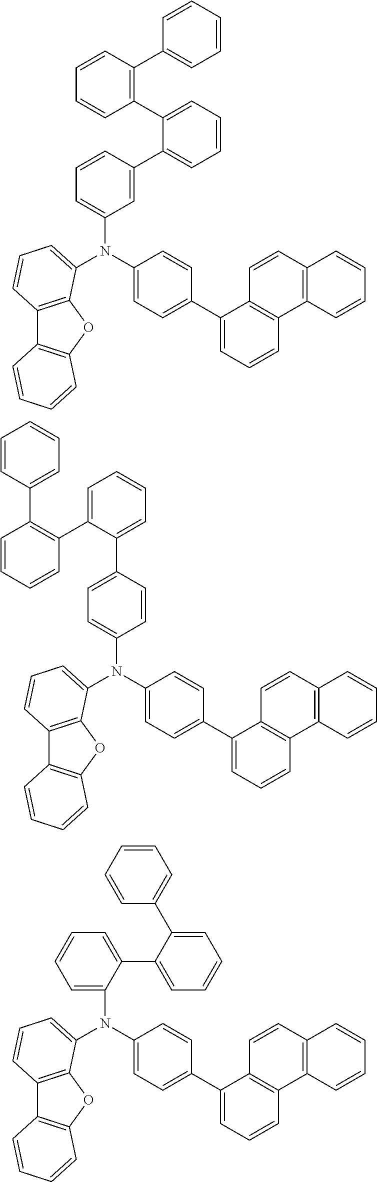

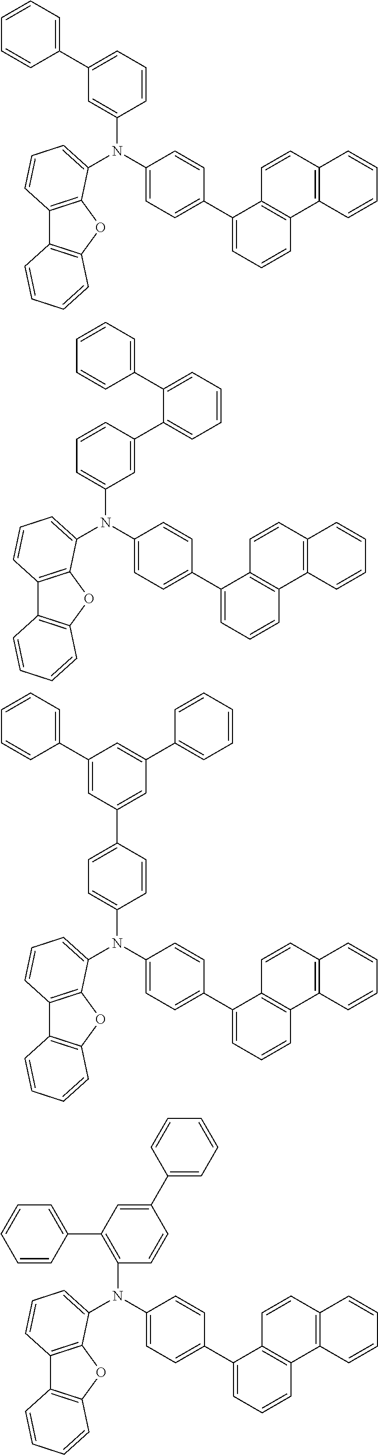

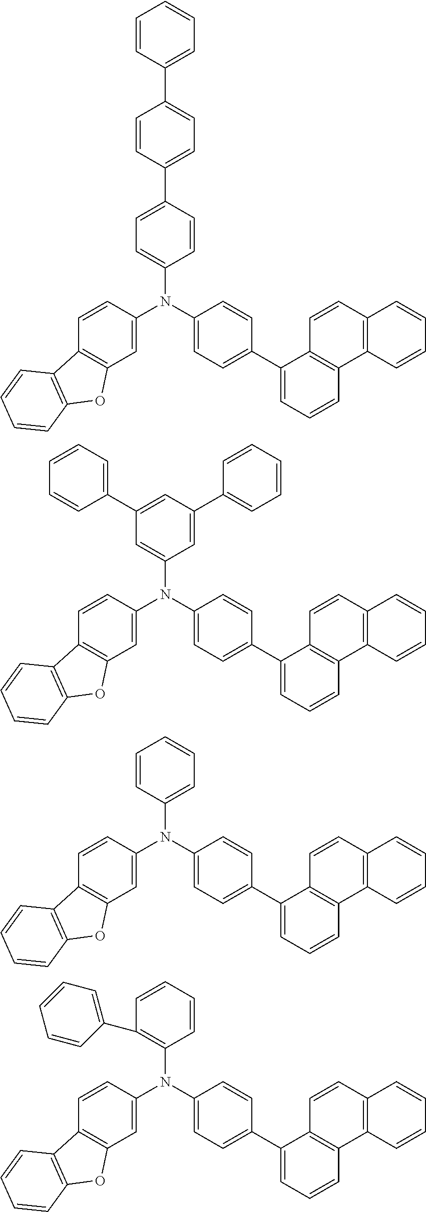

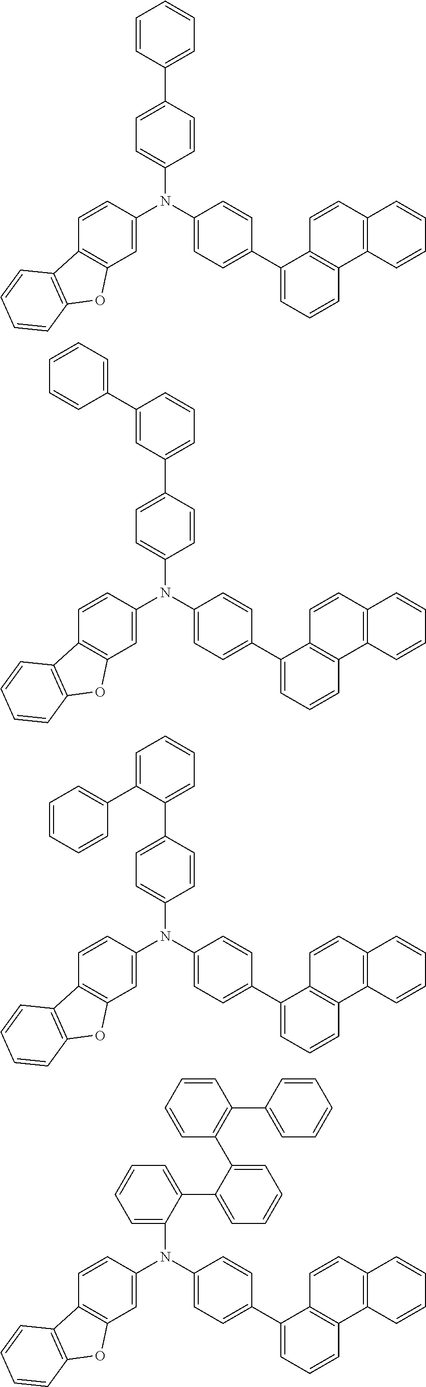

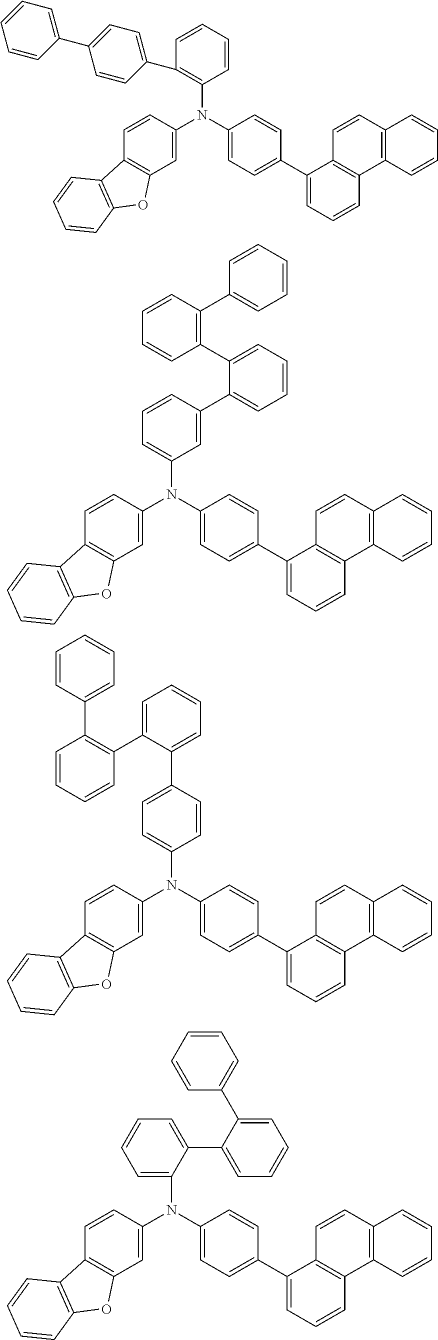

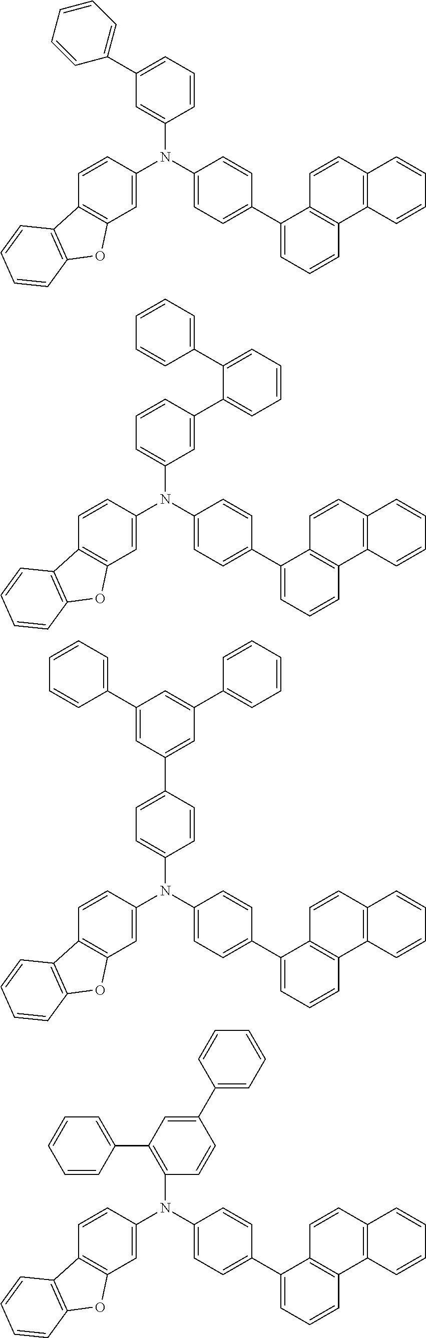

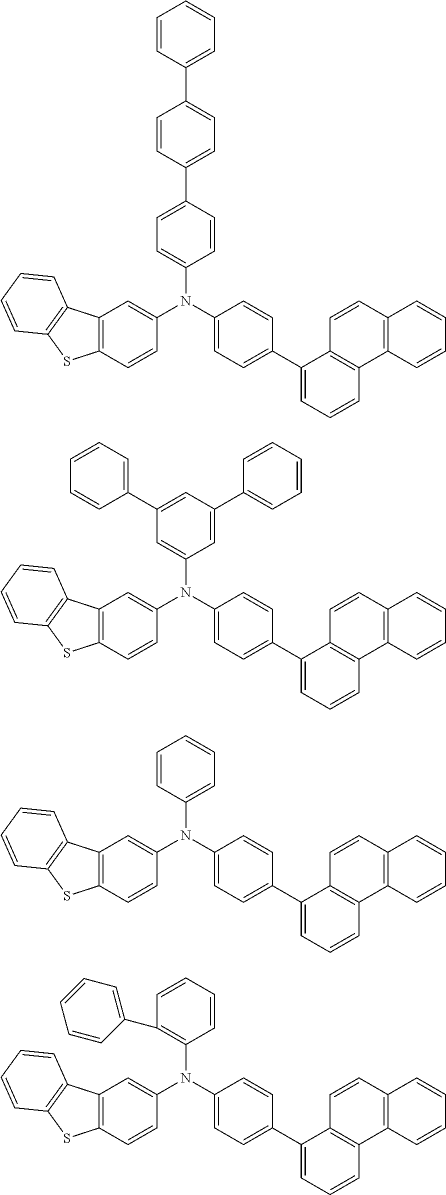 [SMILES: c1ccc(-c2ccc(-c3ccc(N(c4ccc(-c5cccc6c5ccc5ccccc56)cc4)c4ccc5sc6ccccc6c5c4)cc3)cc2)cc1 ; c1ccc(-c2cc(-c3ccccc3)cc(N(c3ccc(-c4cccc5c4ccc4ccccc45)cc3)c3ccc4sc5ccccc5c4c3)c2)cc1 ; c1ccc(N(c2ccc(-c3cccc4c3ccc3ccccc34)cc2)c2ccc3sc4ccccc4c3c2)cc1 ; c1ccc(-c2ccccc2N(c2ccc(-c3cccc4c3ccc3ccccc34)cc2)c2ccc3sc4ccccc4c3c2)cc1]

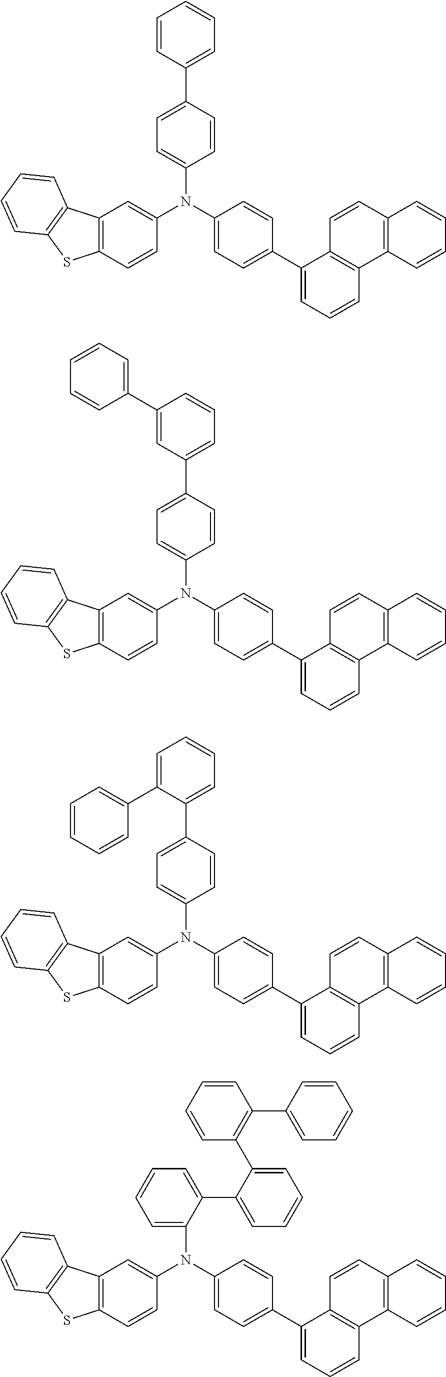

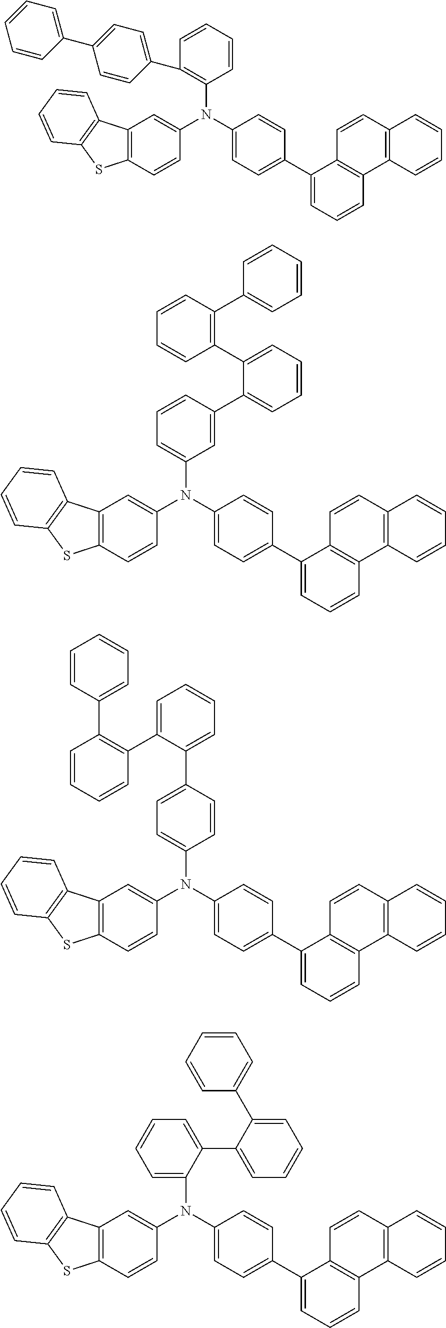 [SMILES: c1ccc(-c2ccc(-c3ccccc3N(c3ccc(-c4cccc5c4ccc4ccccc45)cc3)c3ccc4sc5ccccc5c4c3)cc2)cc1 ; c1ccc(-c2ccccc2-c2ccccc2-c2cccc(N(c3ccc(-c4cccc5c4ccc4ccccc45)cc3)c3ccc4sc5ccccc5c4c3)c2)cc1 ; c1ccc(-c2ccccc2-c2ccccc2-c2ccc(N(c3ccc(-c4cccc5c4ccc4ccccc45)cc3)c3ccc4sc5ccccc5c4c3)cc2)cc1 ; c1ccc(-c2ccccc2-c2ccccc2N(c2ccc(-c3cccc4c3ccc3ccccc34)cc2)c2ccc3sc4ccccc4c3c2)cc1]

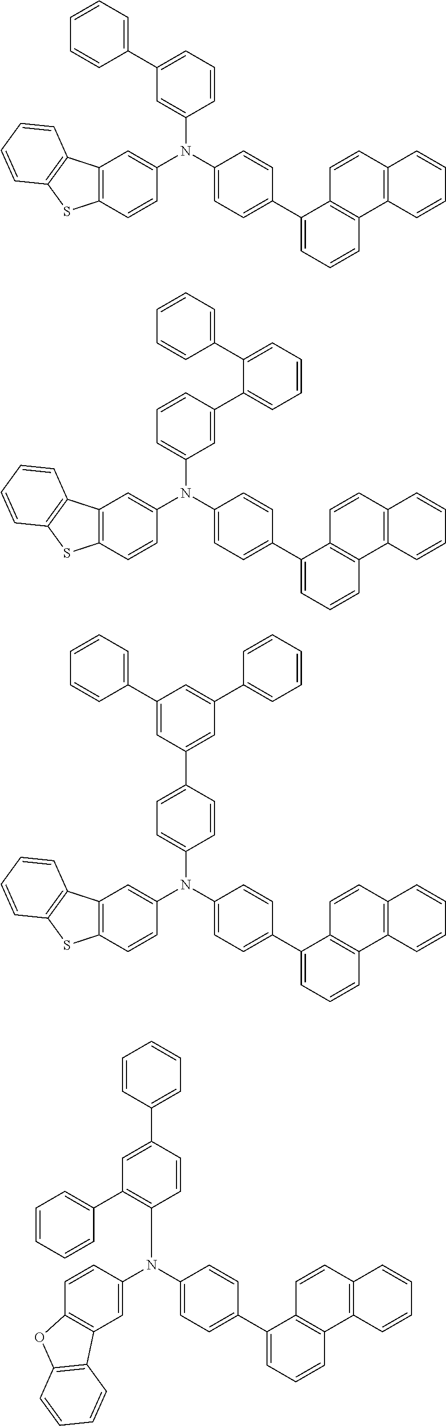 [SMILES: c1ccc(-c2cccc(N(c3ccc(-c4cccc5c4ccc4ccccc45)cc3)c3ccc4sc5ccccc5c4c3)c2)cc1 ; c1ccc(-c2ccccc2-c2cccc(N(c3ccc(-c4cccc5c4ccc4ccccc45)cc3)c3ccc4sc5ccccc5c4c3)c2)cc1 ; c1ccc(-c2cc(-c3ccccc3)cc(-c3ccc(N(c4ccc(-c5cccc6c5ccc5ccccc56)cc4)c4ccc5sc6ccccc6c5c4)cc3)c2)cc1 ; c1ccc(-c2ccc(N(c3ccc(-c4cccc5c4ccc4ccccc45)cc3)c3ccc4oc5ccccc5c4c3)c(-c3ccccc3)c2)cc1]

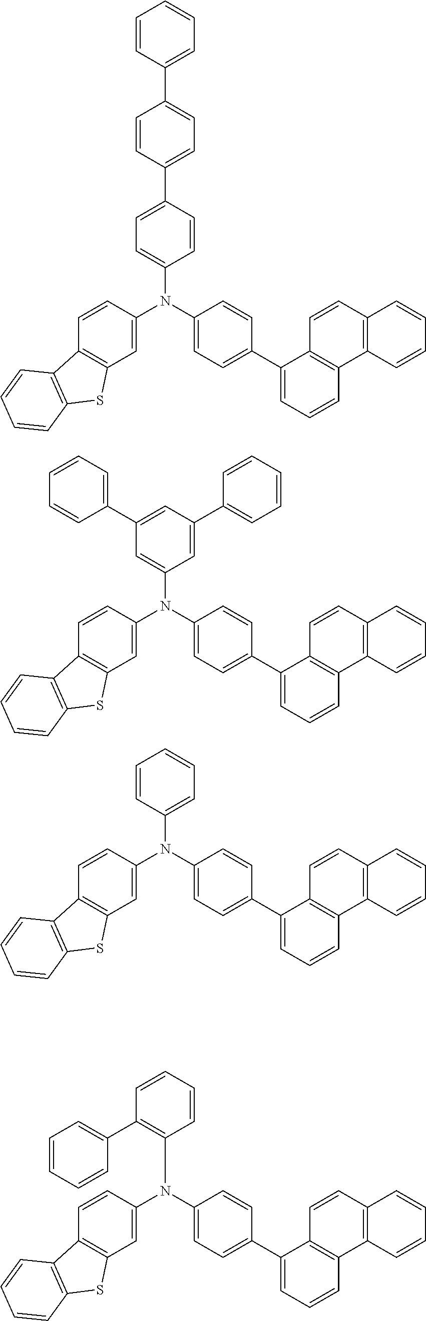

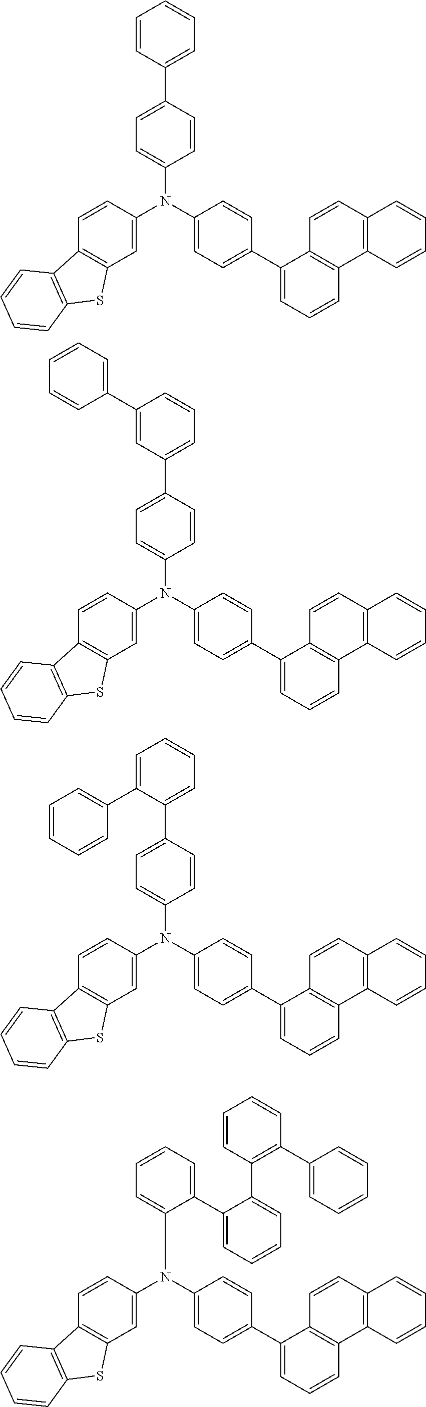

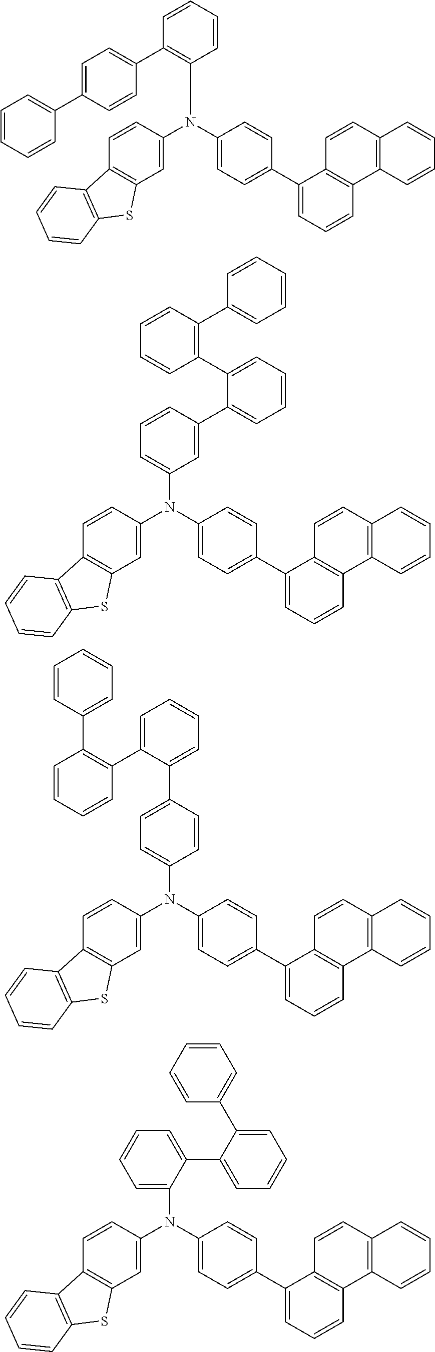

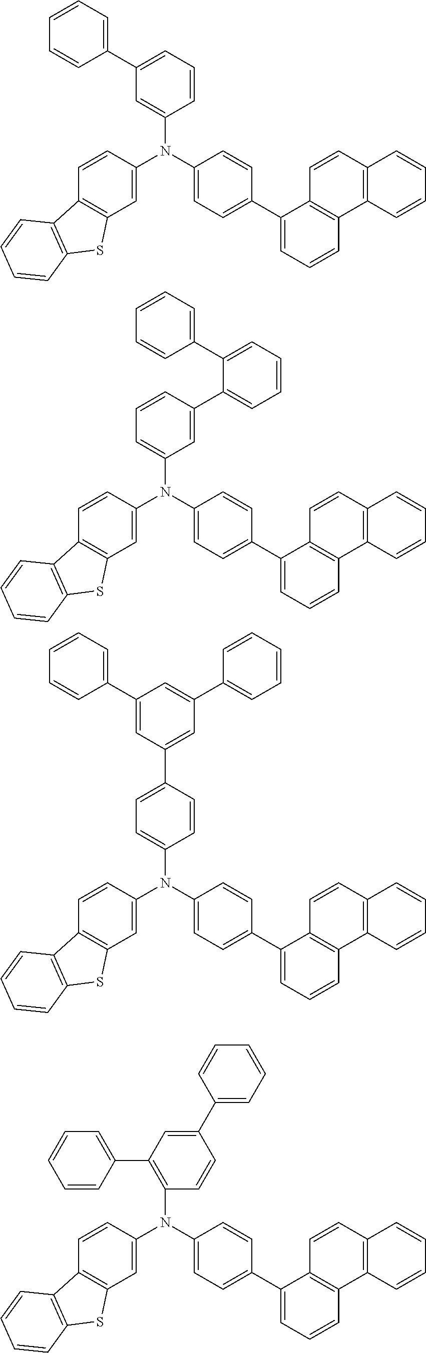

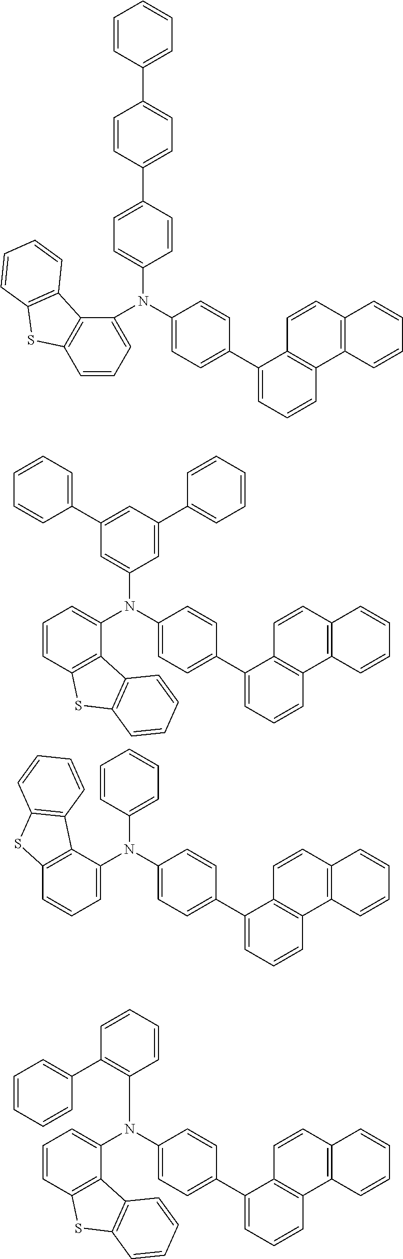

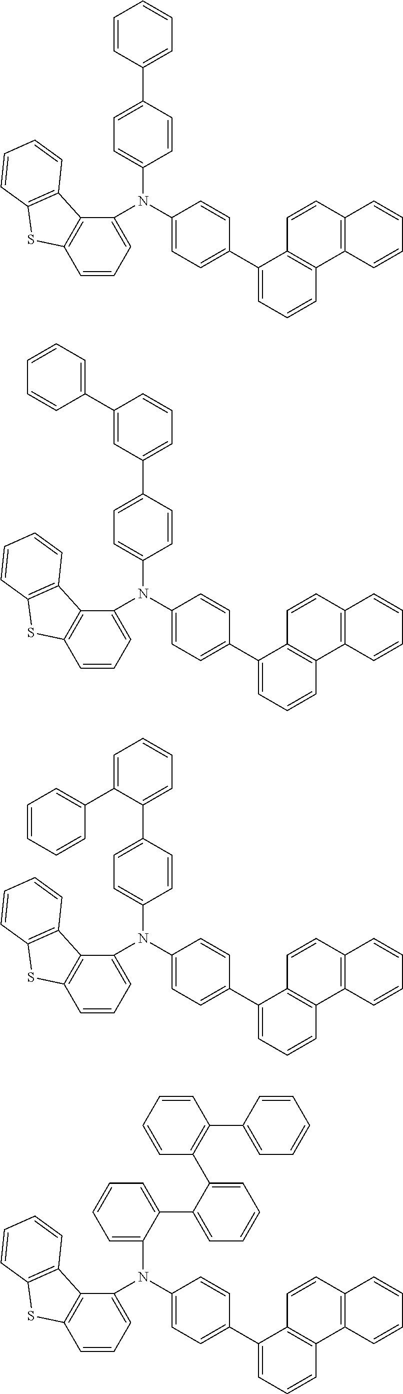

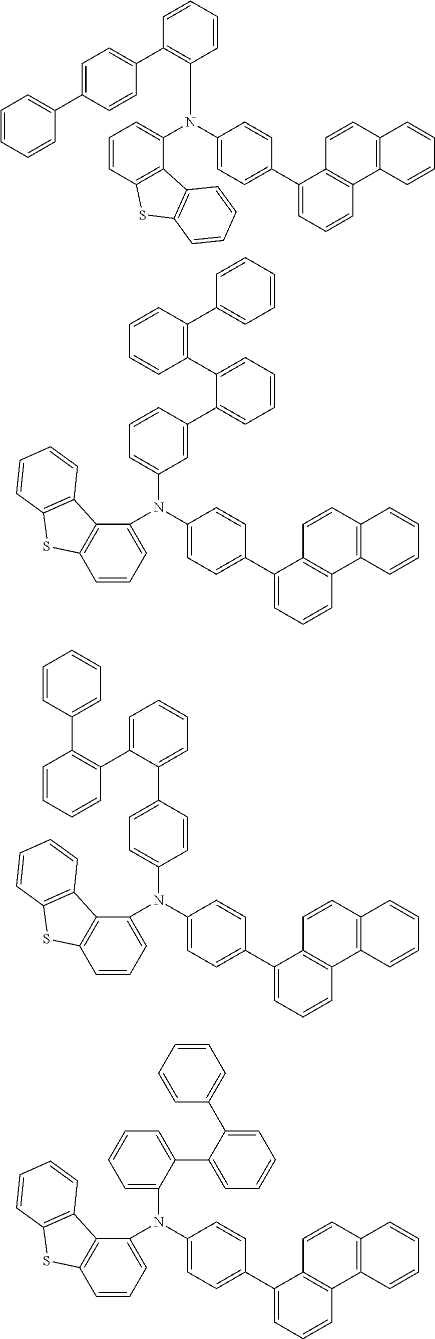

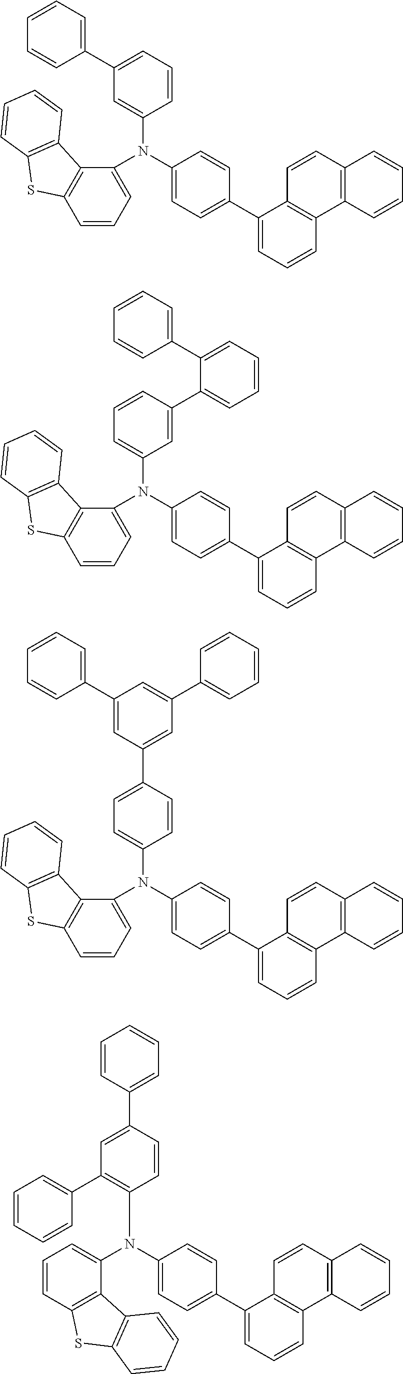 [SMILES: c1ccc(-c2cccc(N(c3ccc(-c4cccc5c4ccc4ccccc45)cc3)c3cccc4sc5ccccc5c34)c2)cc1 ; c1ccc(-c2ccccc2-c2cccc(N(c3ccc(-c4cccc5c4ccc4ccccc45)cc3)c3cccc4sc5ccccc5c34)c2)cc1 ; c1ccc(-c2cc(-c3ccccc3)cc(-c3ccc(N(c4ccc(-c5cccc6c5ccc5ccccc56)cc4)c4cccc5sc6ccccc6c45)cc3)c2)cc1 ; c1ccc(-c2ccc(N(c3ccc(-c4cccc5c4ccc4ccccc45)cc3)c3cccc4sc5ccccc5c34)c(-c3ccccc3)c2)cc1]

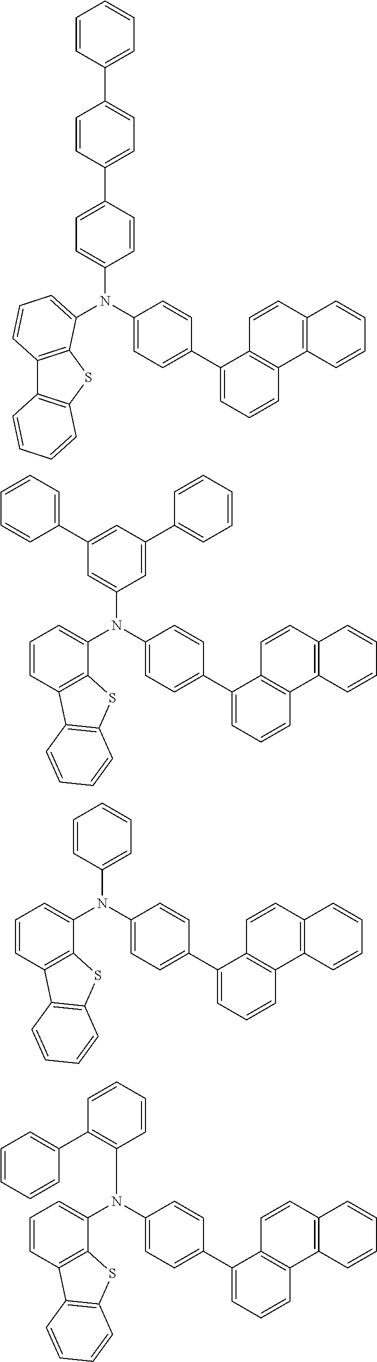

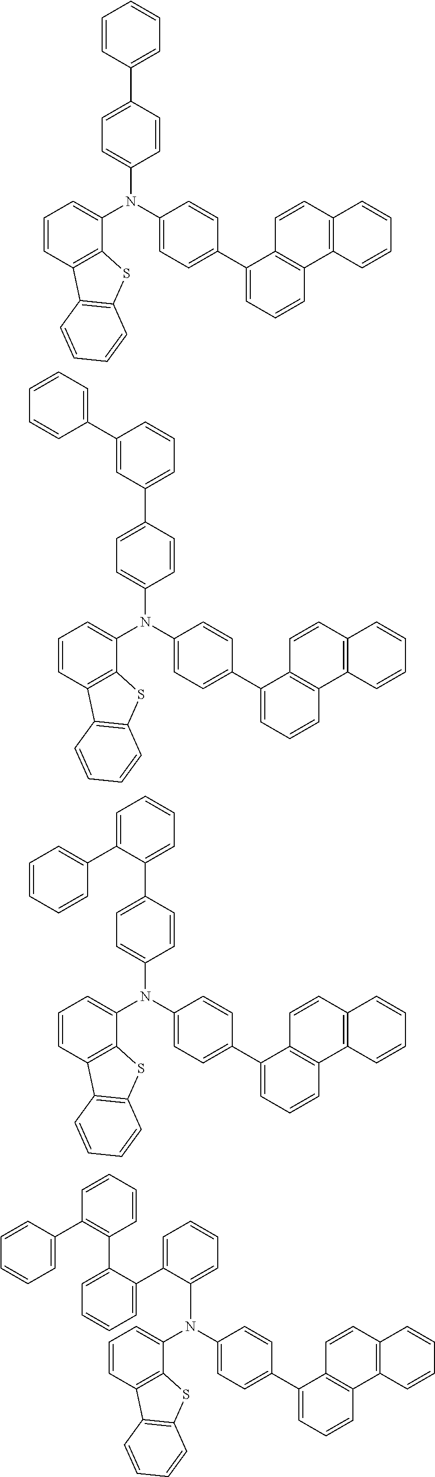

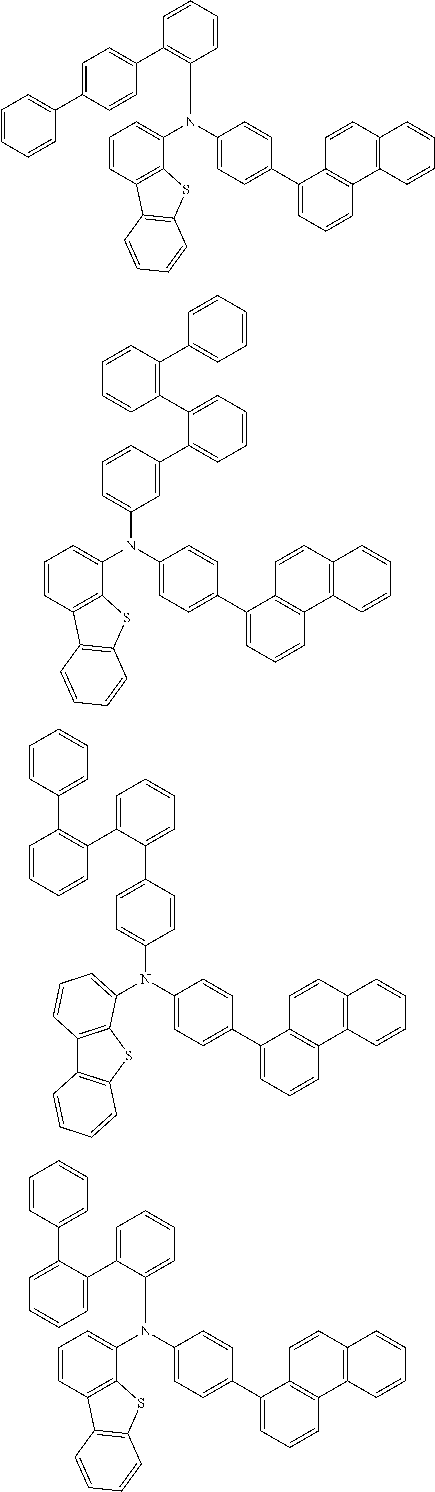

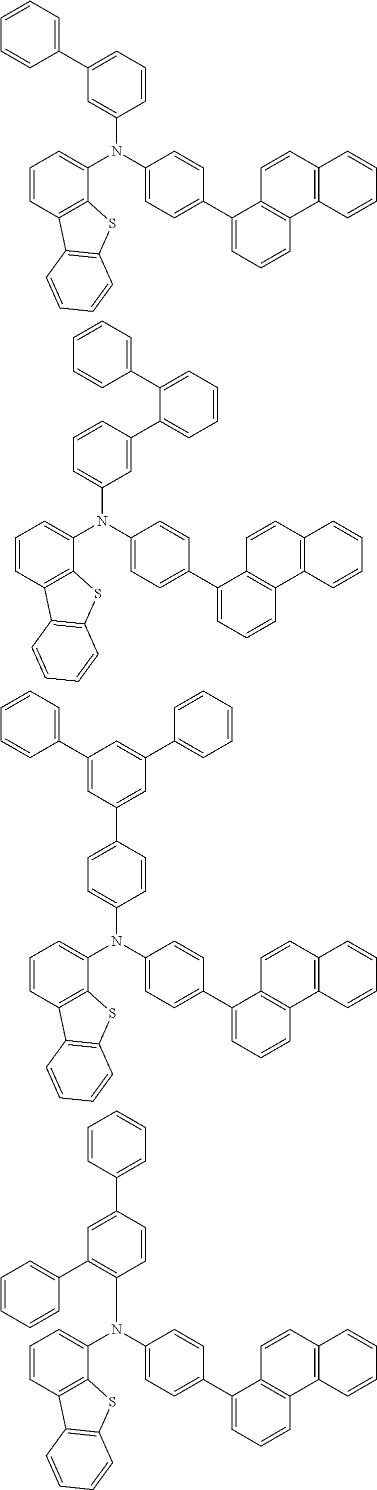

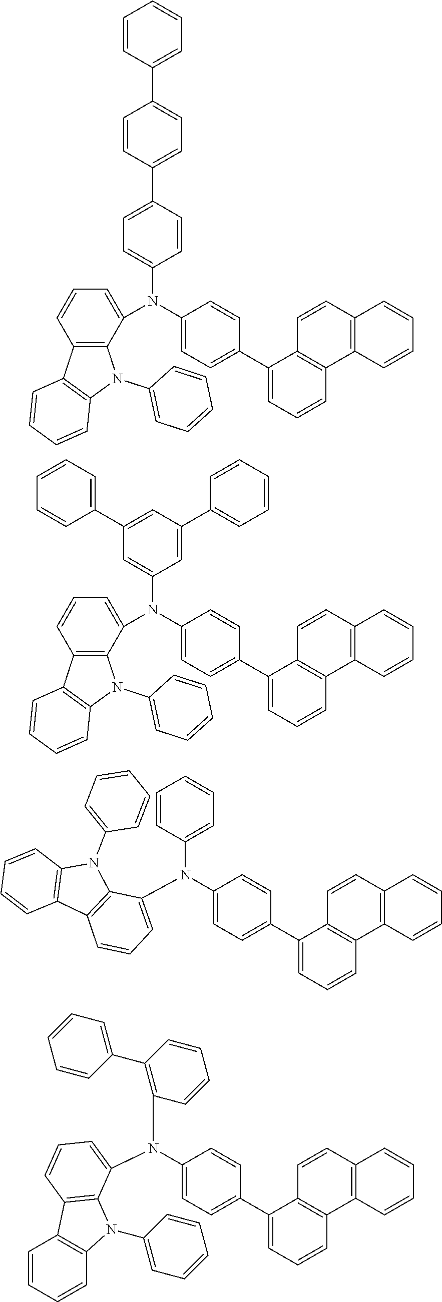

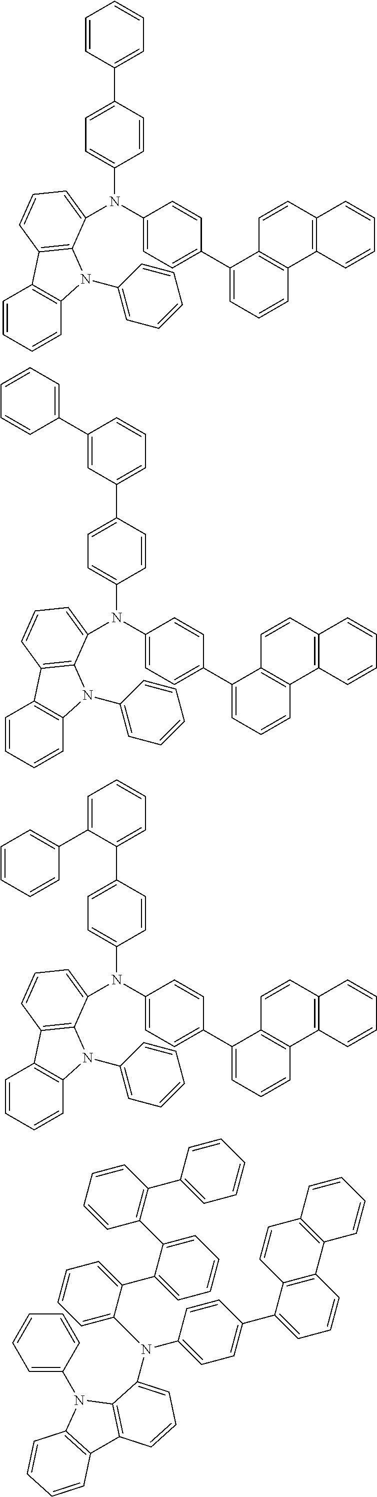

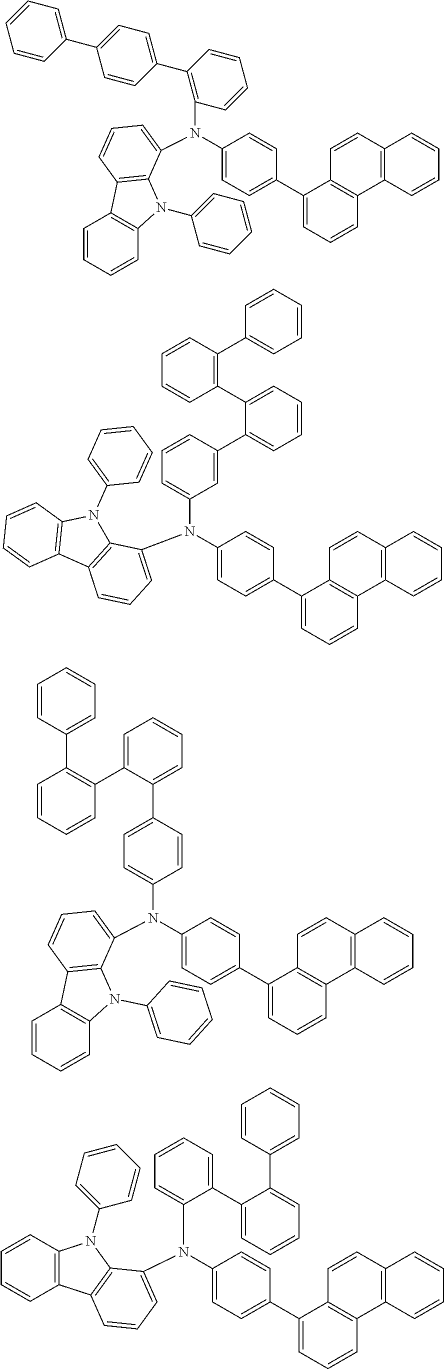 [SMILES: c1ccc(-c2ccc(-c3ccccc3N(c3ccc(-c4cccc5c4ccc4ccccc45)cc3)c3cccc4c5ccccc5n(-c5ccccc5)c34)cc2)cc1 ; c1ccc(-c2ccccc2-c2ccccc2-c2cccc(N(c3ccc(-c4cccc5c4ccc4ccccc45)cc3)c3cccc4c5ccccc5n(-c5ccccc5)c34)c2)cc1 ; c1ccc(-c2ccccc2-c2ccccc2-c2ccc(N(c3ccc(-c4cccc5c4ccc4ccccc45)cc3)c3cccc4c5ccccc5n(-c5ccccc5)c34)cc2)cc1 ; c1ccc(-c2ccccc2-c2ccccc2N(c2ccc(-c3cccc4c3ccc3ccccc34)cc2)c2cccc3c4ccccc4n(-c4ccccc4)c23)cc1]

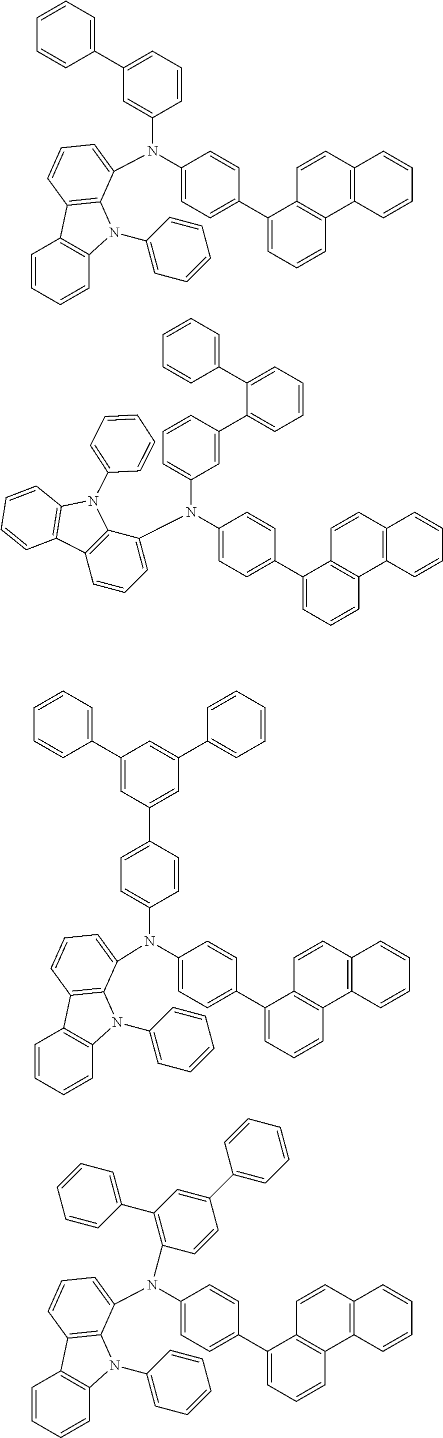

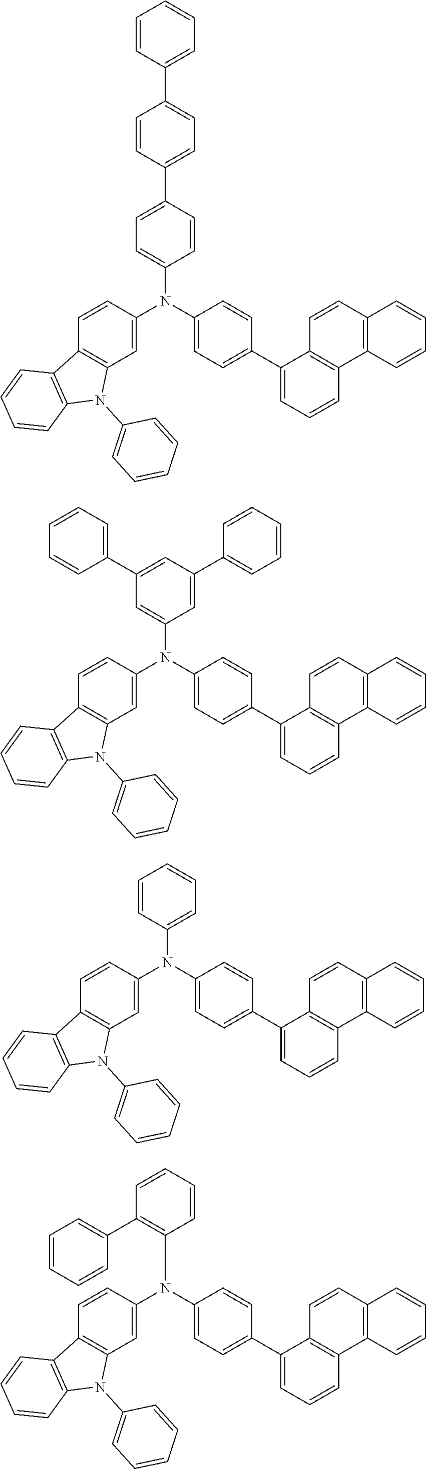

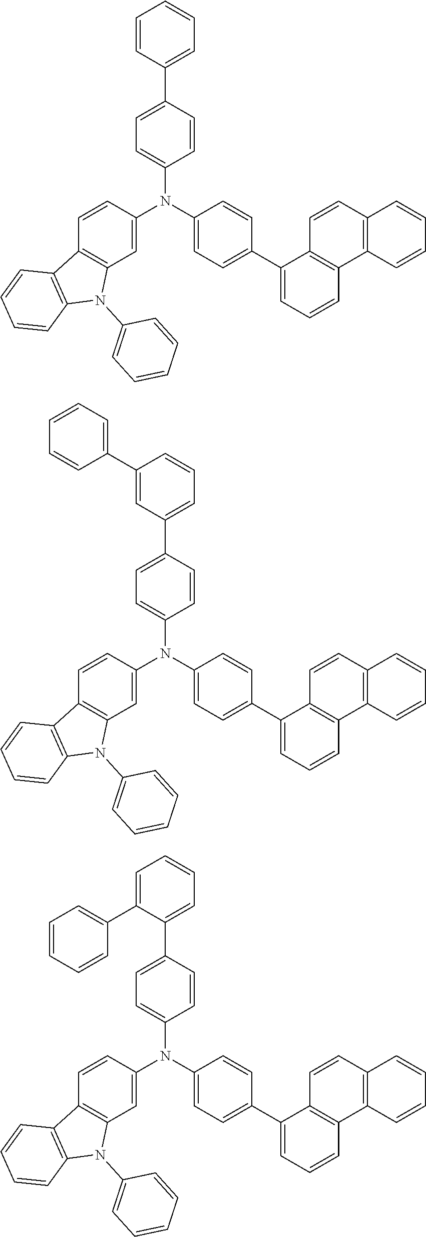

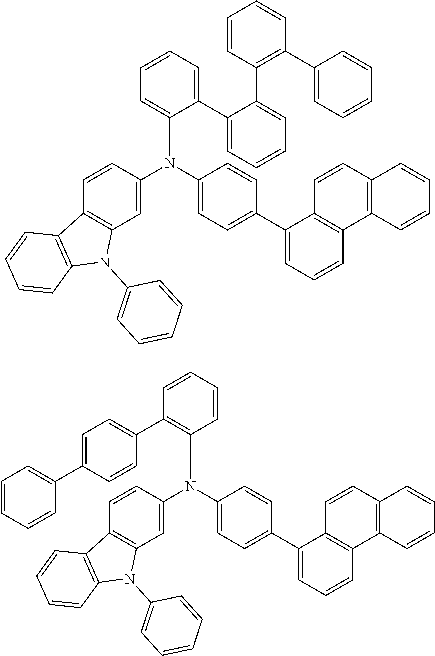

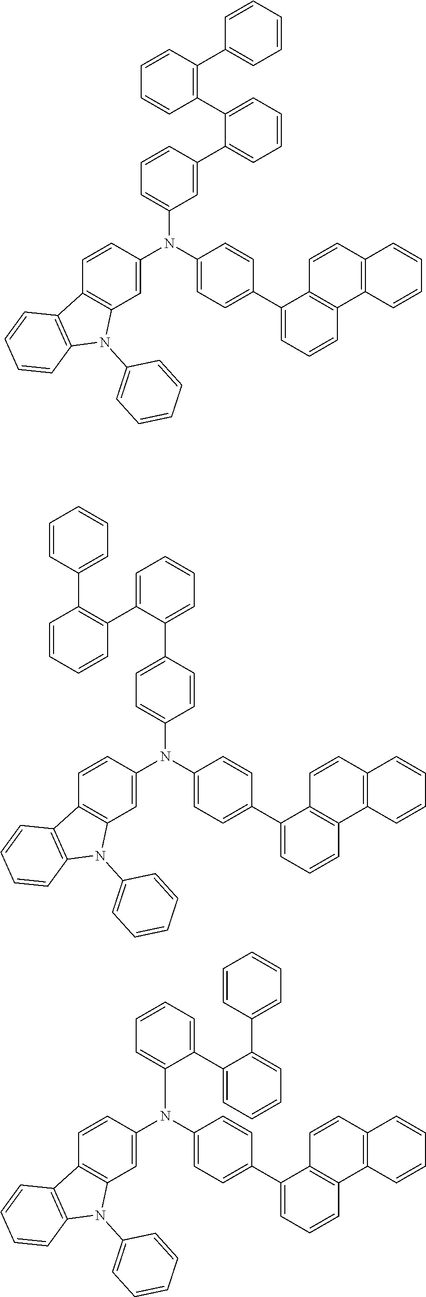 [SMILES: c1ccc(-c2ccccc2-c2ccccc2-c2cccc(N(c3ccc(-c4cccc5c4ccc4ccccc45)cc3)c3ccc4c5ccccc5n(-c5ccccc5)c4c3)c2)cc1 ; c1ccc(-c2ccccc2-c2ccccc2-c2ccc(N(c3ccc(-c4cccc5c4ccc4ccccc45)cc3)c3ccc4c5ccccc5n(-c5ccccc5)c4c3)cc2)cc1 ; c1ccc(-c2ccccc2-c2ccccc2N(c2ccc(-c3cccc4c3ccc3ccccc34)cc2)c2ccc3c4ccccc4n(-c4ccccc4)c3c2)cc1]

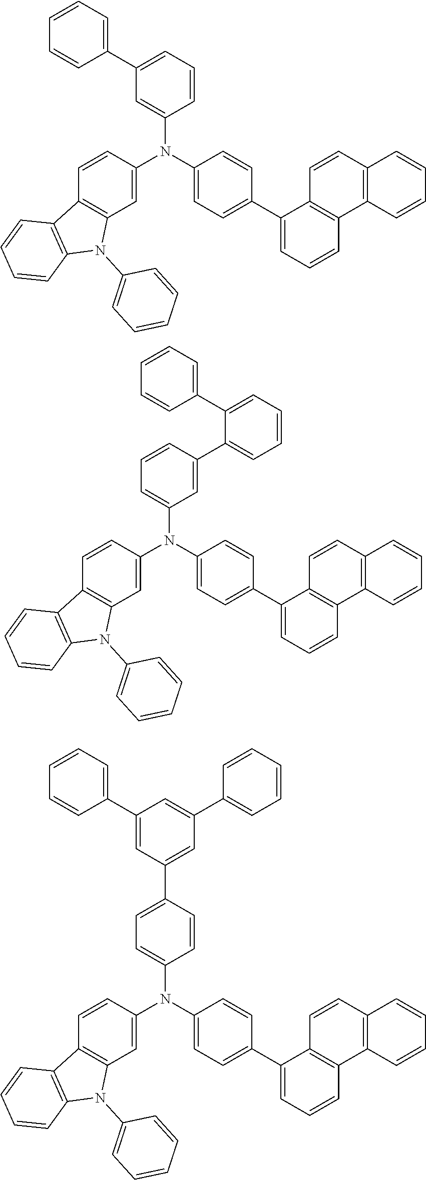

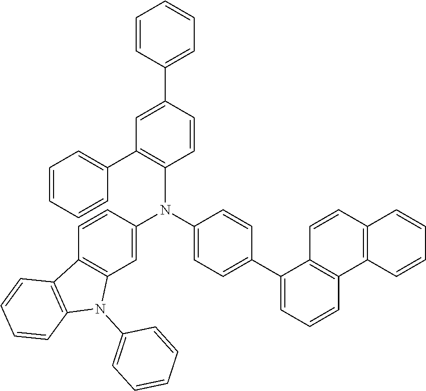 [SMILES: c1ccc(-c2ccc(N(c3ccc(-c4cccc5c4ccc4ccccc45)cc3)c3ccc4c5ccccc5n(-c5ccccc5)c4c3)c(-c3ccccc3)c2)cc1]

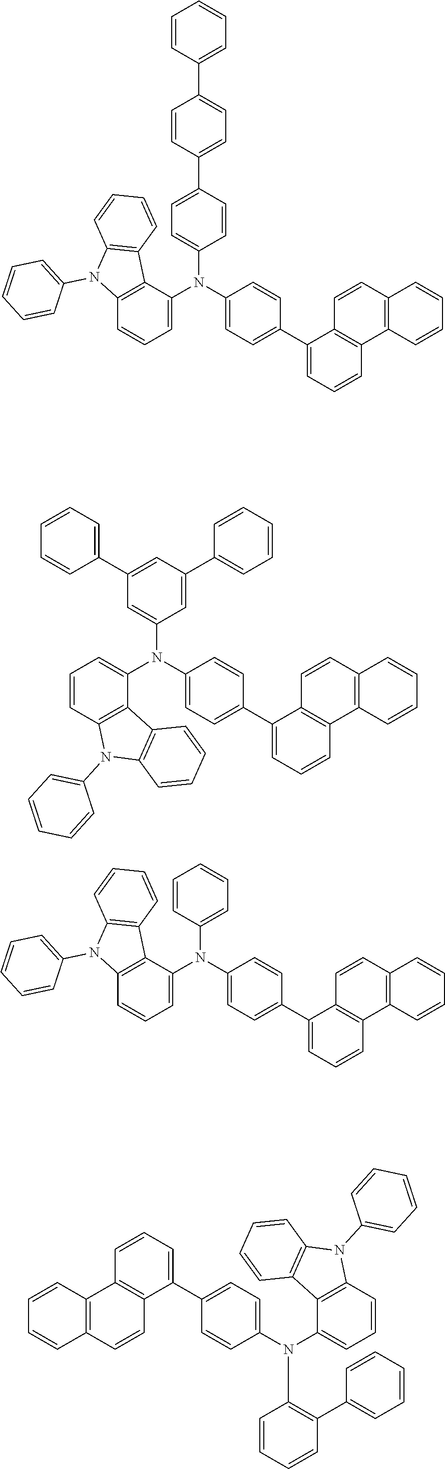 [SMILES: c1ccc(-c2ccc(-c3ccc(N(c4ccc(-c5cccc6c5ccc5ccccc56)cc4)c4cccc5c4c4ccccc4n5-c4ccccc4)cc3)cc2)cc1 ; c1ccc(-c2cc(-c3ccccc3)cc(N(c3ccc(-c4cccc5c4ccc4ccccc45)cc3)c3cccc4c3c3ccccc3n4-c3ccccc3)c2)cc1 ; c1ccc(N(c2ccc(-c3cccc4c3ccc3ccccc34)cc2)c2cccc3c2c2ccccc2n3-c2ccccc2)cc1 ; c1ccc(-c2ccccc2N(c2ccc(-c3cccc4c3ccc3ccccc34)cc2)c2cccc3c2c2ccccc2n3-c2ccccc2)cc1]

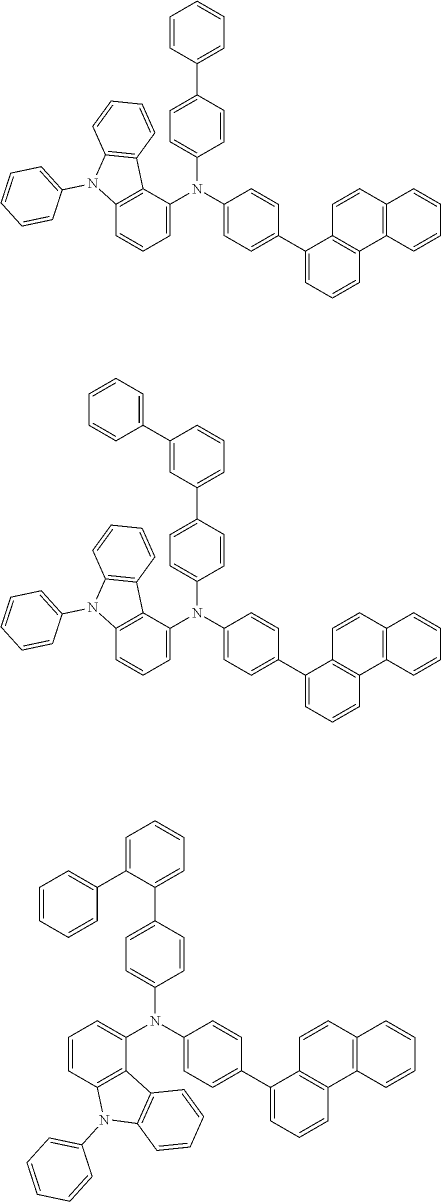 [SMILES: c1ccc(-c2ccc(N(c3ccc(-c4cccc5c4ccc4ccccc45)cc3)c3cccc4c3c3ccccc3n4-c3ccccc3)cc2)cc1 ; c1ccc(-c2cccc(-c3ccc(N(c4ccc(-c5cccc6c5ccc5ccccc56)cc4)c4cccc5c4c4ccccc4n5-c4ccccc4)cc3)c2)cc1 ; c1ccc(-c2ccccc2-c2ccc(N(c3ccc(-c4cccc5c4ccc4ccccc45)cc3)c3cccc4c3c3ccccc3n4-c3ccccc3)cc2)cc1]

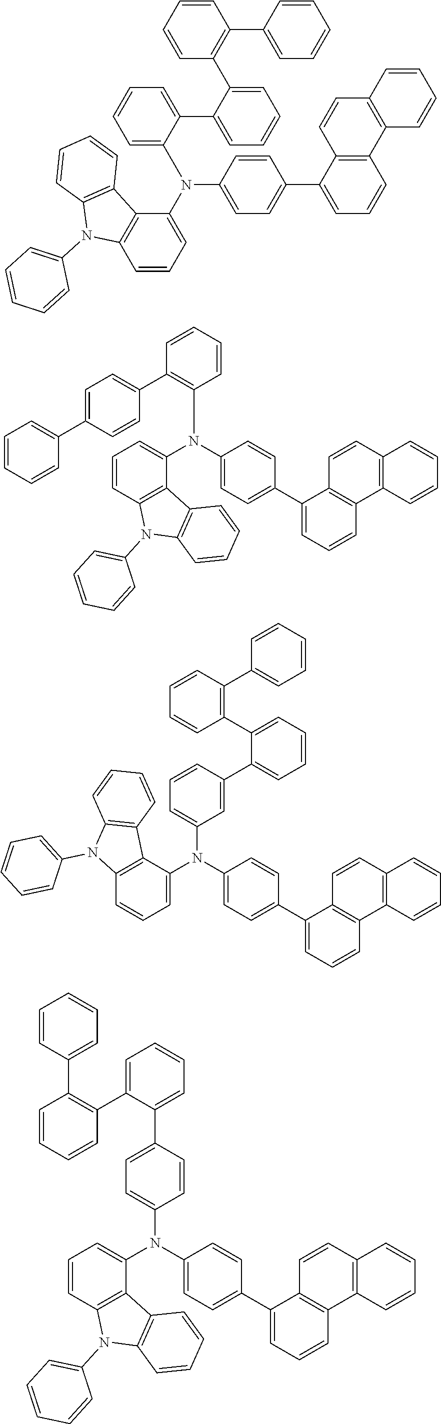 [SMILES: c1ccc(-c2ccccc2-c2ccccc2-c2ccccc2N(c2ccc(-c3cccc4c3ccc3ccccc34)cc2)c2cccc3c2c2ccccc2n3-c2ccccc2)cc1 ; c1ccc(-c2ccc(-c3ccccc3N(c3ccc(-c4cccc5c4ccc4ccccc45)cc3)c3cccc4c3c3ccccc3n4-c3ccccc3)cc2)cc1 ; c1ccc(-c2ccccc2-c2ccccc2-c2cccc(N(c3ccc(-c4cccc5c4ccc4ccccc45)cc3)c3cccc4c3c3ccccc3n4-c3ccccc3)c2)cc1 ; c1ccc(-c2ccccc2-c2ccccc2-c2ccc(N(c3ccc(-c4cccc5c4ccc4ccccc45)cc3)c3cccc4c3c3ccccc3n4-c3ccccc3)cc2)cc1]

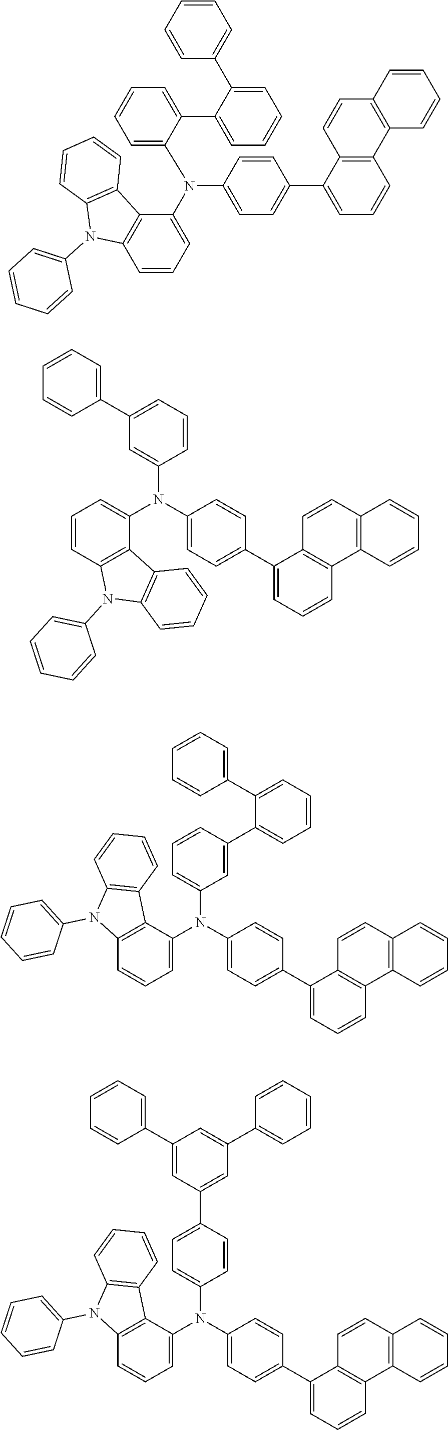

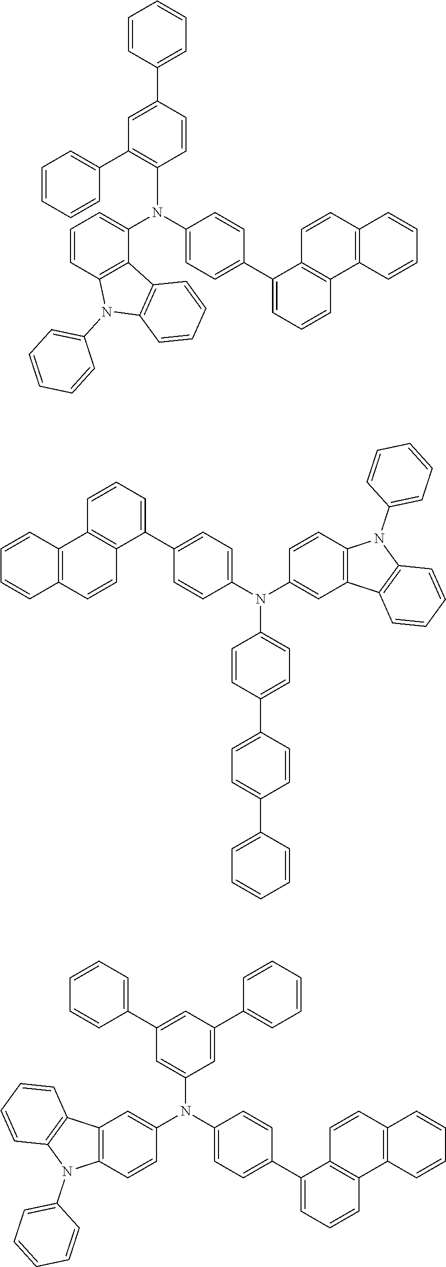 [SMILES: c1ccc(-c2ccc(N(c3ccc(-c4cccc5c4ccc4ccccc45)cc3)c3cccc4c3c3ccccc3n4-c3ccccc3)c(-c3ccccc3)c2)cc1 ; c1ccc(-c2ccc(-c3ccc(N(c4ccc(-c5cccc6c5ccc5ccccc56)cc4)c4ccc5c(c4)c4ccccc4n5-c4ccccc4)cc3)cc2)cc1 ; c1ccc(-c2cc(-c3ccccc3)cc(N(c3ccc(-c4cccc5c4ccc4ccccc45)cc3)c3ccc4c(c3)c3ccccc3n4-c3ccccc3)c2)cc1]

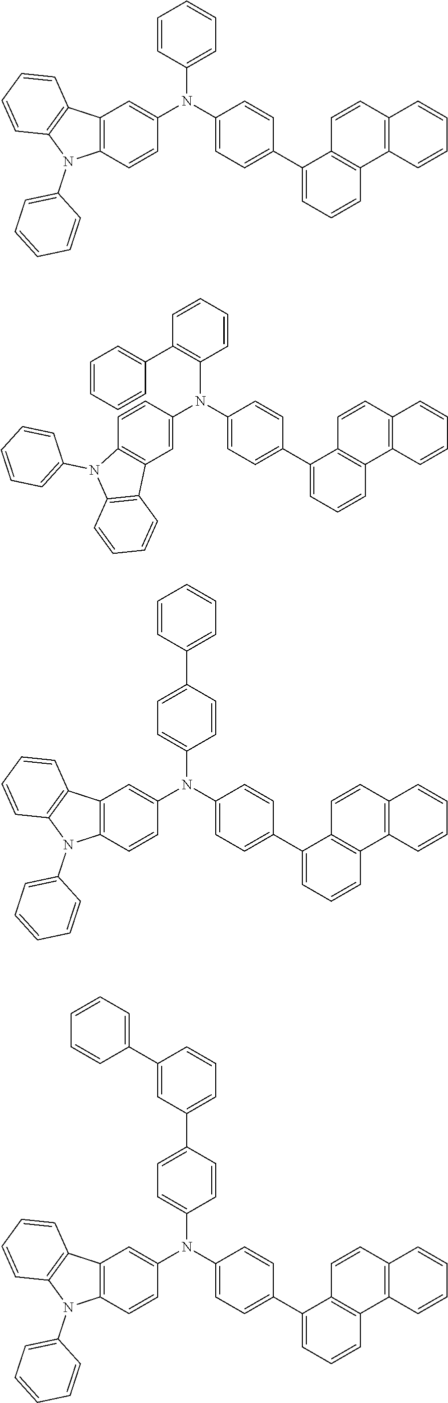 [SMILES: c1ccc(N(c2ccc(-c3cccc4c3ccc3ccccc34)cc2)c2ccc3c(c2)c2ccccc2n3-c2ccccc2)cc1 ; c1ccc(-c2ccccc2N(c2ccc(-c3cccc4c3ccc3ccccc34)cc2)c2ccc3c(c2)c2ccccc2n3-c2ccccc2)cc1 ; c1ccc(-c2ccc(N(c3ccc(-c4cccc5c4ccc4ccccc45)cc3)c3ccc4c(c3)c3ccccc3n4-c3ccccc3)cc2)cc1 ; c1ccc(-c2cccc(-c3ccc(N(c4ccc(-c5cccc6c5ccc5ccccc56)cc4)c4ccc5c(c4)c4ccccc4n5-c4ccccc4)cc3)c2)cc1]

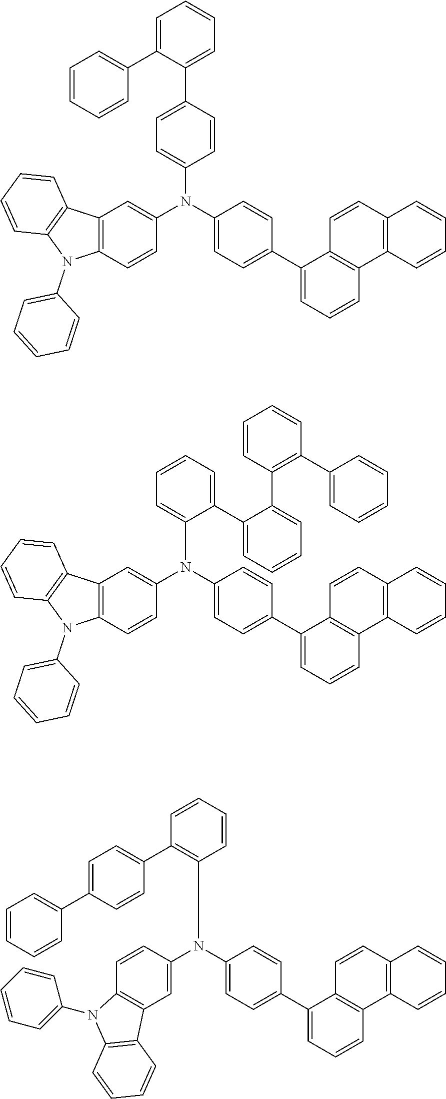 [SMILES: c1ccc(-c2ccccc2-c2ccc(N(c3ccc(-c4cccc5c4ccc4ccccc45)cc3)c3ccc4c(c3)c3ccccc3n4-c3ccccc3)cc2)cc1 ; c1ccc(-c2ccccc2-c2ccccc2-c2ccccc2N(c2ccc(-c3cccc4c3ccc3ccccc34)cc2)c2ccc3c(c2)c2ccccc2n3-c2ccccc2)cc1 ; c1ccc(-c2ccc(-c3ccccc3N(c3ccc(-c4cccc5c4ccc4ccccc45)cc3)c3ccc4c(c3)c3ccccc3n4-c3ccccc3)cc2)cc1]

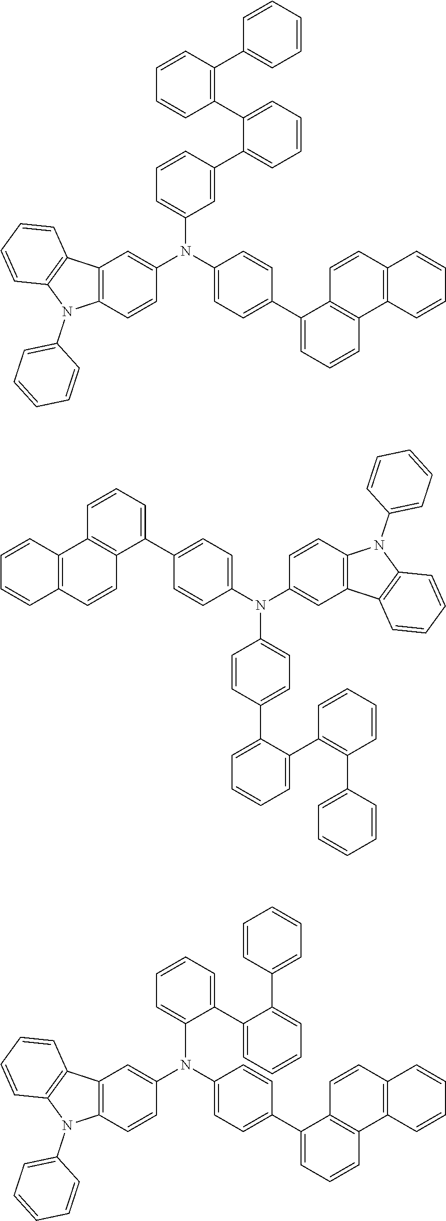 [SMILES: c1ccc(-c2ccccc2-c2ccccc2-c2cccc(N(c3ccc(-c4cccc5c4ccc4ccccc45)cc3)c3ccc4c(c3)c3ccccc3n4-c3ccccc3)c2)cc1 ; c1ccc(-c2ccccc2-c2ccccc2-c2ccc(N(c3ccc(-c4cccc5c4ccc4ccccc45)cc3)c3ccc4c(c3)c3ccccc3n4-c3ccccc3)cc2)cc1 ; c1ccc(-c2ccccc2-c2ccccc2N(c2ccc(-c3cccc4c3ccc3ccccc34)cc2)c2ccc3c(c2)c2ccccc2n3-c2ccccc2)cc1]

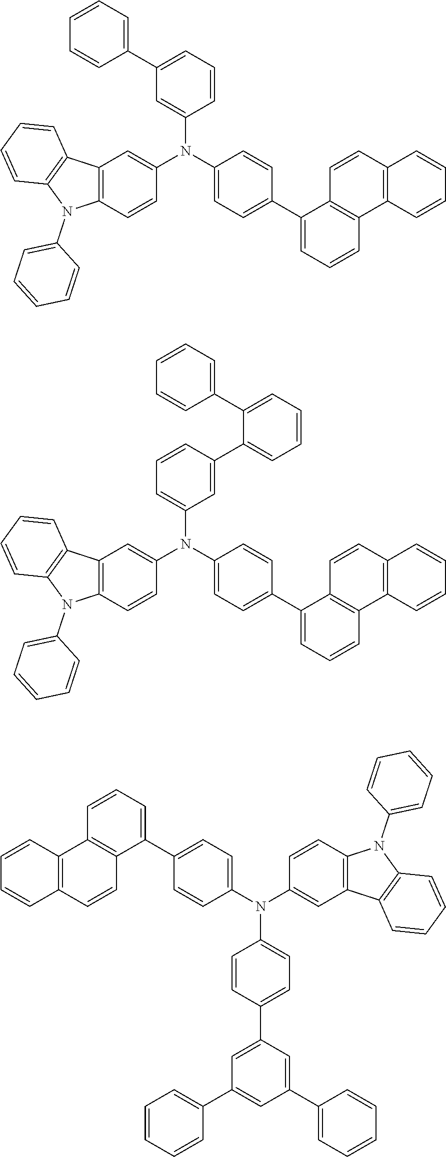 [SMILES: c1ccc(-c2cccc(N(c3ccc(-c4cccc5c4ccc4ccccc45)cc3)c3ccc4c(c3)c3ccccc3n4-c3ccccc3)c2)cc1 ; c1ccc(-c2ccccc2-c2cccc(N(c3ccc(-c4cccc5c4ccc4ccccc45)cc3)c3ccc4c(c3)c3ccccc3n4-c3ccccc3)c2)cc1 ; c1ccc(-c2cc(-c3ccccc3)cc(-c3ccc(N(c4ccc(-c5cccc6c5ccc5ccccc56)cc4)c4ccc5c(c4)c4ccccc4n5-c4ccccc4)cc3)c2)cc1]

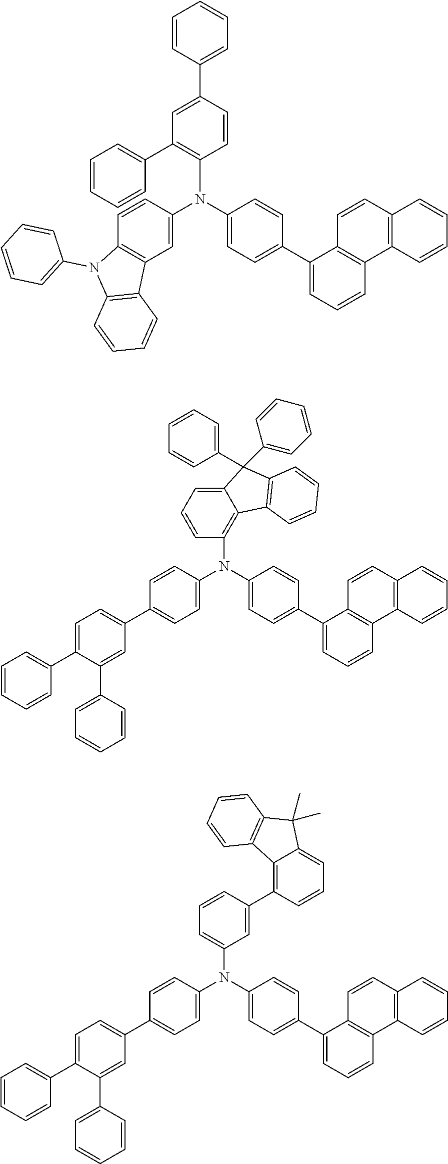 [SMILES: c1ccc(-c2ccc(N(c3ccc(-c4cccc5c4ccc4ccccc45)cc3)c3ccc4c(c3)c3ccccc3n4-c3ccccc3)c(-c3ccccc3)c2)cc1 ; c1ccc(-c2ccc(-c3ccc(N(c4ccc(-c5cccc6c5ccc5ccccc56)cc4)c4cccc5c4-c4ccccc4C5(c4ccccc4)c4ccccc4)cc3)cc2-c2ccccc2)cc1 ; CC1(C)c2ccccc2-c2c(-c3cccc(N(c4ccc(-c5ccc(-c6ccccc6)c(-c6ccccc6)c5)cc4)c4ccc(-c5cccc6c5ccc5ccccc56)cc4)c3)cccc21]

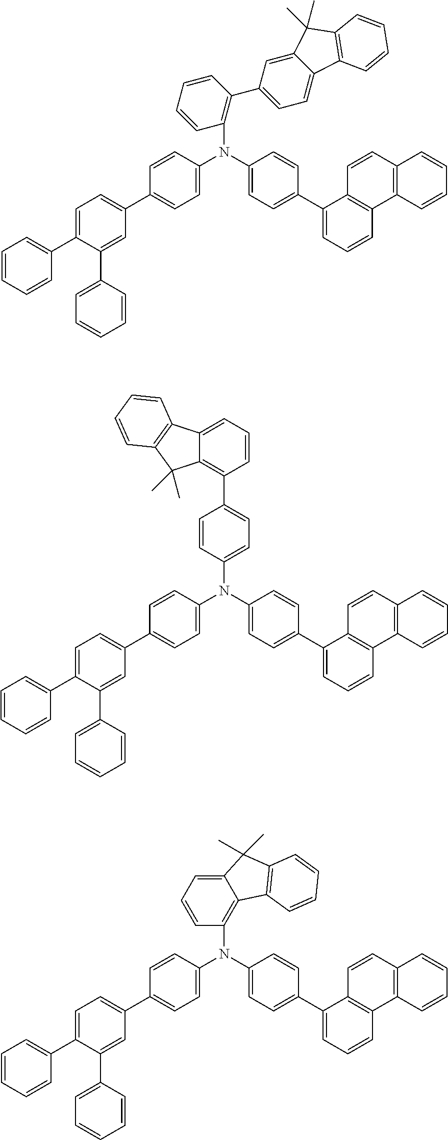 [SMILES: CC1(C)c2ccccc2-c2ccc(-c3ccccc3N(c3ccc(-c4ccc(-c5ccccc5)c(-c5ccccc5)c4)cc3)c3ccc(-c4cccc5c4ccc4ccccc45)cc3)cc21 ; CC1(C)c2ccccc2-c2cccc(-c3ccc(N(c4ccc(-c5ccc(-c6ccccc6)c(-c6ccccc6)c5)cc4)c4ccc(-c5cccc6c5ccc5ccccc56)cc4)cc3)c21 ; CC1(C)c2ccccc2-c2c(N(c3ccc(-c4ccc(-c5ccccc5)c(-c5ccccc5)c4)cc3)c3ccc(-c4cccc5c4ccc4ccccc45)cc3)cccc21]

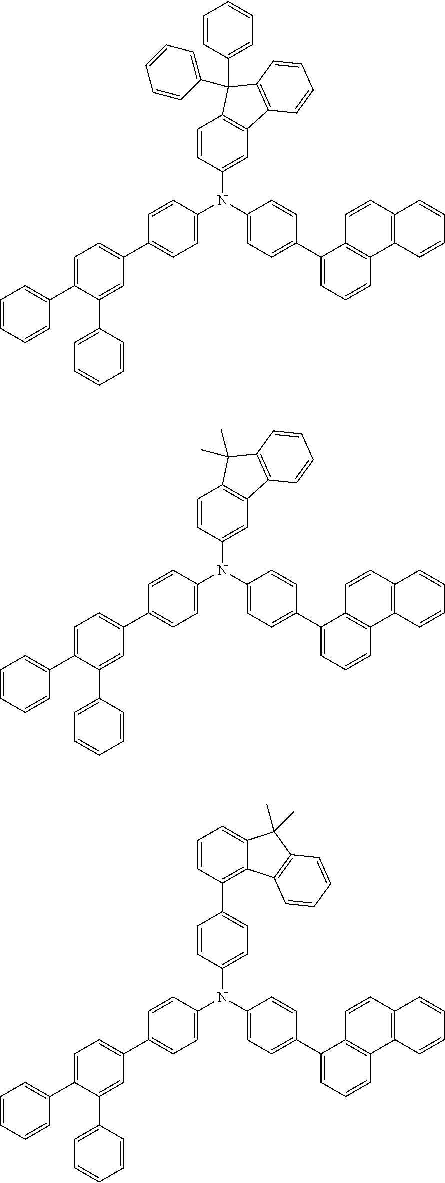 [SMILES: c1ccc(-c2ccc(-c3ccc(N(c4ccc(-c5cccc6c5ccc5ccccc56)cc4)c4ccc5c(c4)-c4ccccc4C5(c4ccccc4)c4ccccc4)cc3)cc2-c2ccccc2)cc1 ; CC1(C)c2ccccc2-c2cc(N(c3ccc(-c4ccc(-c5ccccc5)c(-c5ccccc5)c4)cc3)c3ccc(-c4cccc5c4ccc4ccccc45)cc3)ccc21 ; CC1(C)c2ccccc2-c2c(-c3ccc(N(c4ccc(-c5ccc(-c6ccccc6)c(-c6ccccc6)c5)cc4)c4ccc(-c5cccc6c5ccc5ccccc56)cc4)cc3)cccc21]

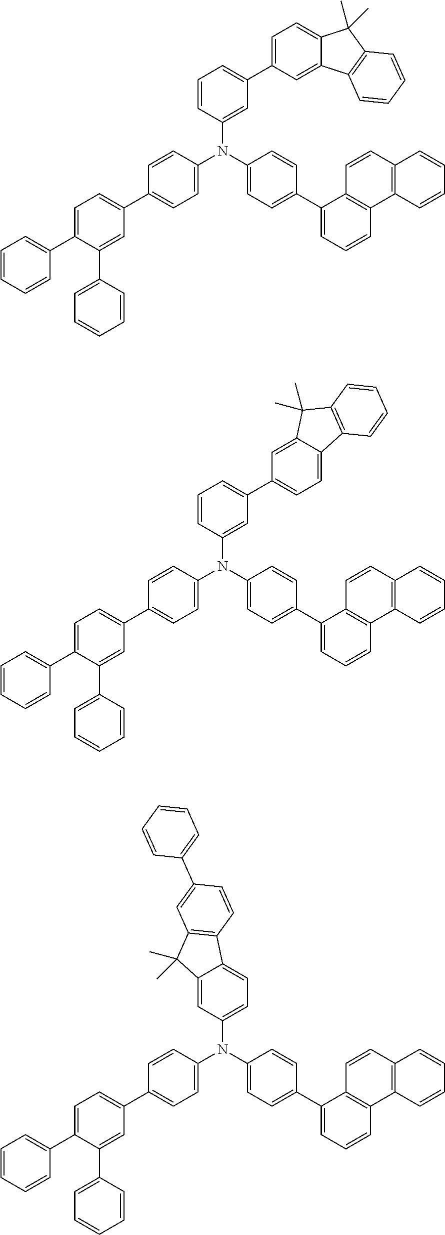 [SMILES: CC1(C)c2ccccc2-c2cc(-c3cccc(N(c4ccc(-c5ccc(-c6ccccc6)c(-c6ccccc6)c5)cc4)c4ccc(-c5cccc6c5ccc5ccccc56)cc4)c3)ccc21 ; CC1(C)c2ccccc2-c2ccc(-c3cccc(N(c4ccc(-c5ccc(-c6ccccc6)c(-c6ccccc6)c5)cc4)c4ccc(-c5cccc6c5ccc5ccccc56)cc4)c3)cc21 ; CC1(C)c2cc(-c3ccccc3)ccc2-c2ccc(N(c3ccc(-c4ccc(-c5ccccc5)c(-c5ccccc5)c4)cc3)c3ccc(-c4cccc5c4ccc4ccccc45)cc3)cc21]

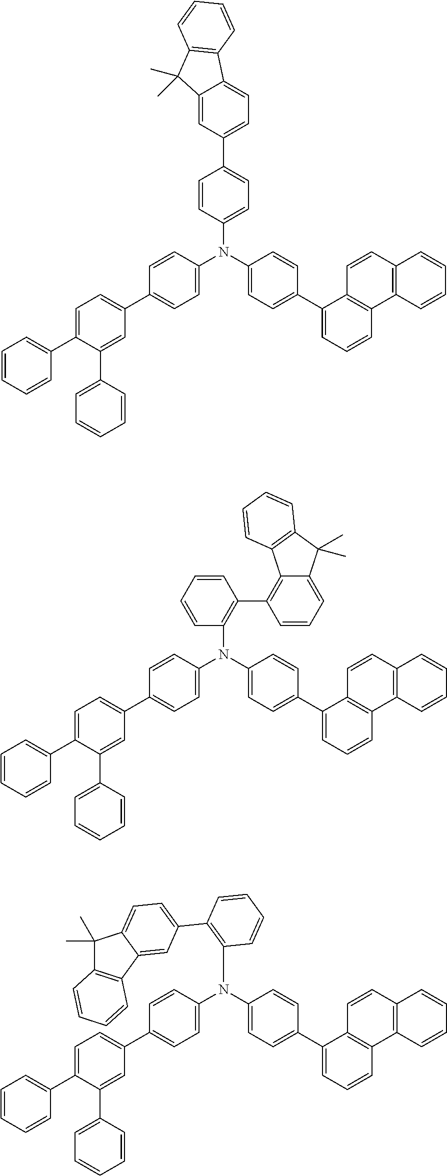 [SMILES: CC1(C)c2ccccc2-c2ccc(-c3ccc(N(c4ccc(-c5ccc(-c6ccccc6)c(-c6ccccc6)c5)cc4)c4ccc(-c5cccc6c5ccc5ccccc56)cc4)cc3)cc21 ; CC1(C)c2ccccc2-c2c(-c3ccccc3N(c3ccc(-c4ccc(-c5ccccc5)c(-c5ccccc5)c4)cc3)c3ccc(-c4cccc5c4ccc4ccccc45)cc3)cccc21 ; CC1(C)c2ccccc2-c2cc(-c3ccccc3N(c3ccc(-c4ccc(-c5ccccc5)c(-c5ccccc5)c4)cc3)c3ccc(-c4cccc5c4ccc4ccccc45)cc3)ccc21]

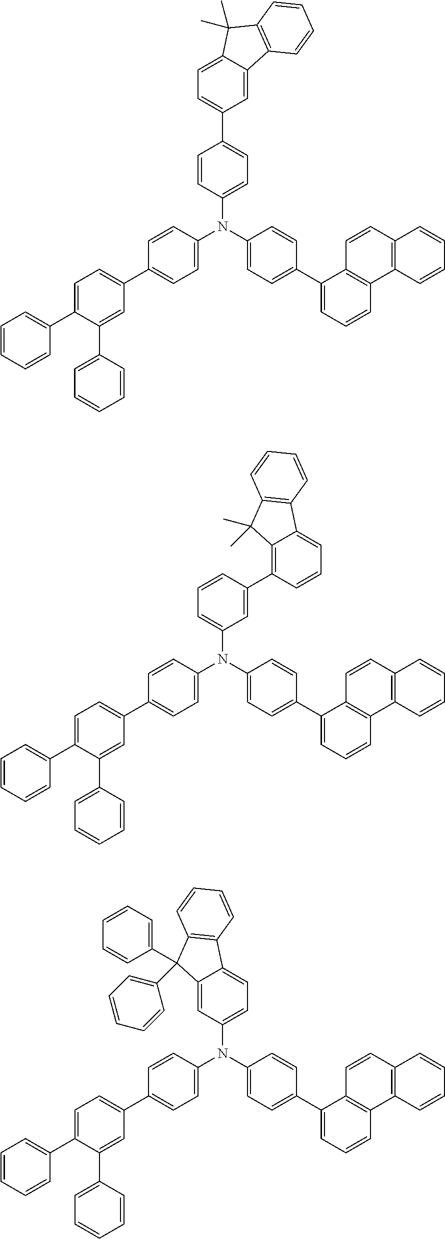 [SMILES: CC1(C)c2ccccc2-c2cc(-c3ccc(N(c4ccc(-c5ccc(-c6ccccc6)c(-c6ccccc6)c5)cc4)c4ccc(-c5cccc6c5ccc5ccccc56)cc4)cc3)ccc21 ; CC1(C)c2ccccc2-c2cccc(-c3cccc(N(c4ccc(-c5ccc(-c6ccccc6)c(-c6ccccc6)c5)cc4)c4ccc(-c5cccc6c5ccc5ccccc56)cc4)c3)c21 ; c1ccc(-c2ccc(-c3ccc(N(c4ccc(-c5cccc6c5ccc5ccccc56)cc4)c4ccc5c(c4)C(c4ccccc4)(c4ccccc4)c4ccccc4-5)cc3)cc2-c2ccccc2)cc1]

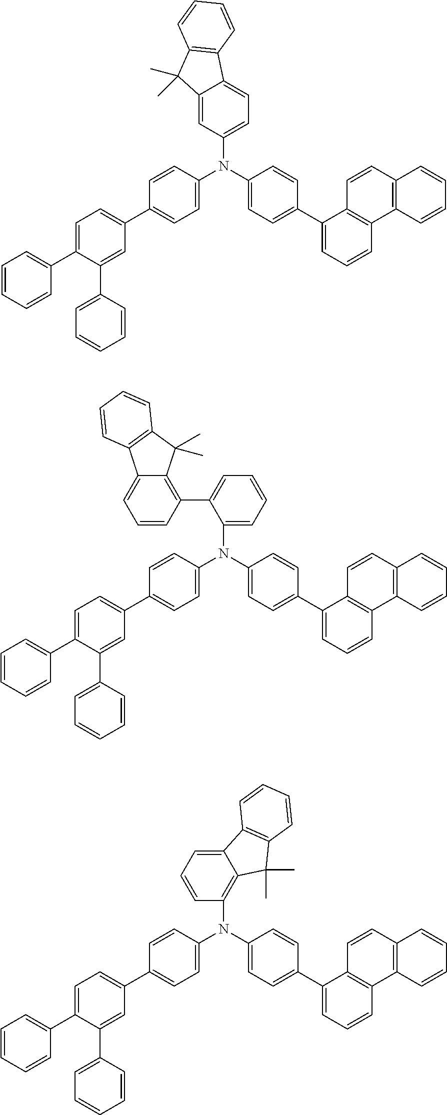 [SMILES: CC1(C)c2ccccc2-c2ccc(N(c3ccc(-c4ccc(-c5ccccc5)c(-c5ccccc5)c4)cc3)c3ccc(-c4cccc5c4ccc4ccccc45)cc3)cc21 ; CC1(C)c2ccccc2-c2cccc(-c3ccccc3N(c3ccc(-c4ccc(-c5ccccc5)c(-c5ccccc5)c4)cc3)c3ccc(-c4cccc5c4ccc4ccccc45)cc3)c21 ; CC1(C)c2ccccc2-c2cccc(N(c3ccc(-c4ccc(-c5ccccc5)c(-c5ccccc5)c4)cc3)c3ccc(-c4cccc5c4ccc4ccccc45)cc3)c21]

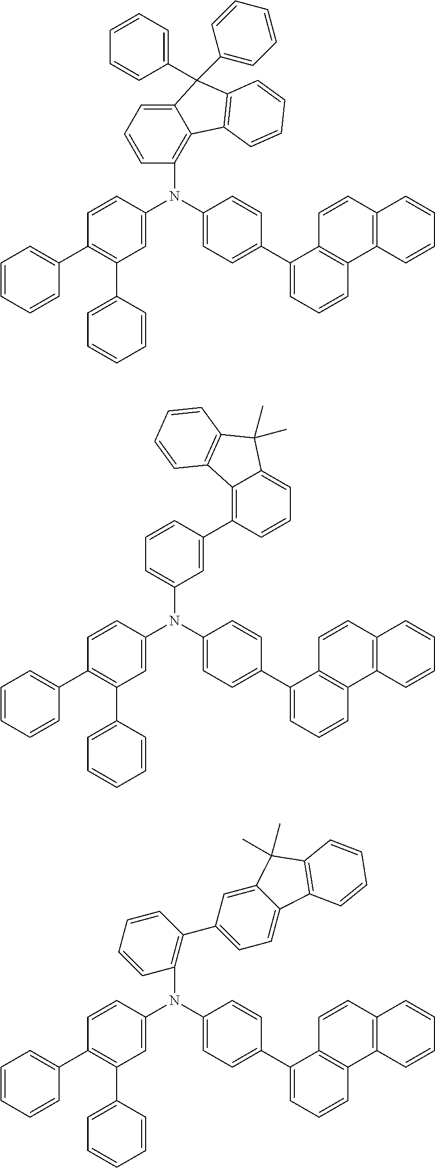 [SMILES: c1ccc(-c2ccc(N(c3ccc(-c4cccc5c4ccc4ccccc45)cc3)c3cccc4c3-c3ccccc3C4(c3ccccc3)c3ccccc3)cc2-c2ccccc2)cc1 ; CC1(C)c2ccccc2-c2c(-c3cccc(N(c4ccc(-c5cccc6c5ccc5ccccc56)cc4)c4ccc(-c5ccccc5)c(-c5ccccc5)c4)c3)cccc21 ; CC1(C)c2ccccc2-c2ccc(-c3ccccc3N(c3ccc(-c4cccc5c4ccc4ccccc45)cc3)c3ccc(-c4ccccc4)c(-c4ccccc4)c3)cc21]

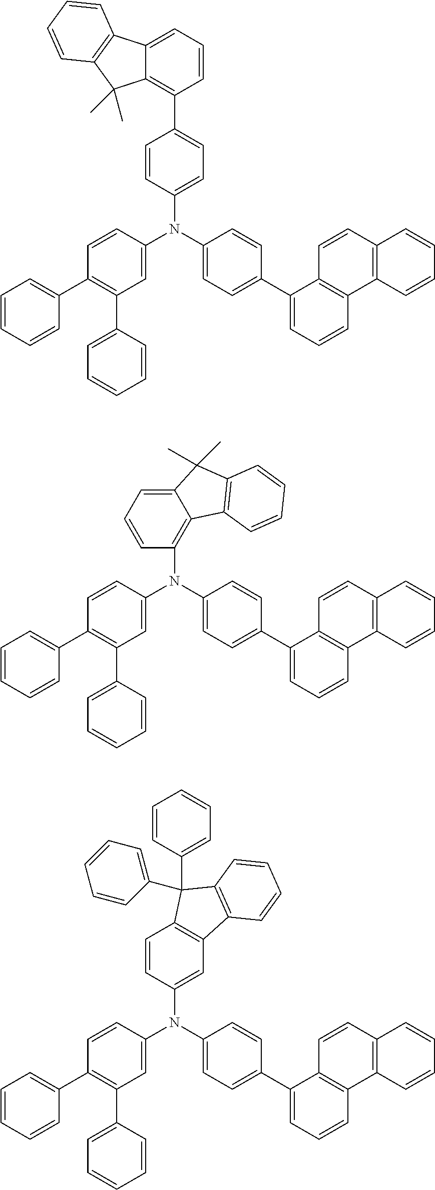 [SMILES: CC1(C)c2ccccc2-c2cccc(-c3ccc(N(c4ccc(-c5cccc6c5ccc5ccccc56)cc4)c4ccc(-c5ccccc5)c(-c5ccccc5)c4)cc3)c21 ; CC1(C)c2ccccc2-c2c(N(c3ccc(-c4cccc5c4ccc4ccccc45)cc3)c3ccc(-c4ccccc4)c(-c4ccccc4)c3)cccc21 ; c1ccc(-c2ccc(N(c3ccc(-c4cccc5c4ccc4ccccc45)cc3)c3ccc4c(c3)-c3ccccc3C4(c3ccccc3)c3ccccc3)cc2-c2ccccc2)cc1]

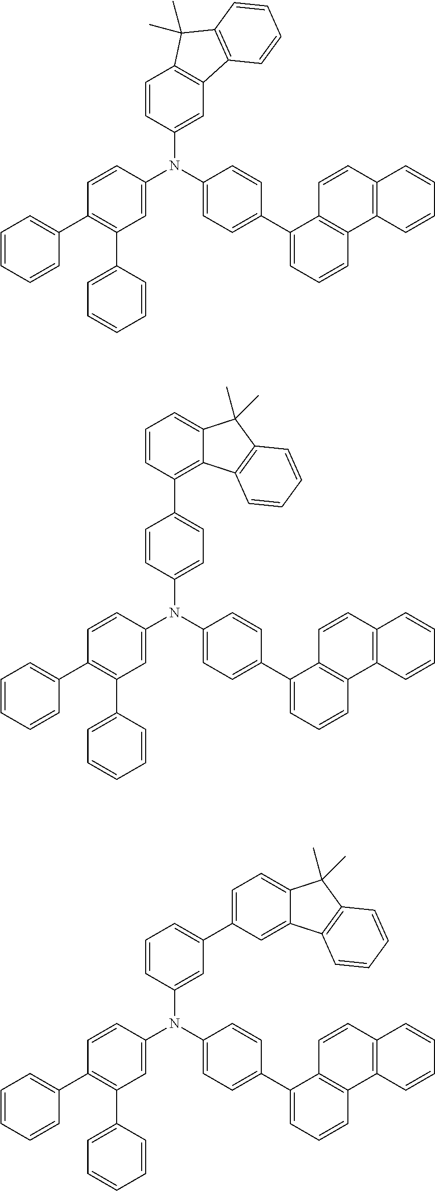 [SMILES: CC1(C)c2ccccc2-c2cc(N(c3ccc(-c4cccc5c4ccc4ccccc45)cc3)c3ccc(-c4ccccc4)c(-c4ccccc4)c3)ccc21 ; CC1(C)c2ccccc2-c2c(-c3ccc(N(c4ccc(-c5cccc6c5ccc5ccccc56)cc4)c4ccc(-c5ccccc5)c(-c5ccccc5)c4)cc3)cccc21 ; CC1(C)c2ccccc2-c2cc(-c3cccc(N(c4ccc(-c5cccc6c5ccc5ccccc56)cc4)c4ccc(-c5ccccc5)c(-c5ccccc5)c4)c3)ccc21]

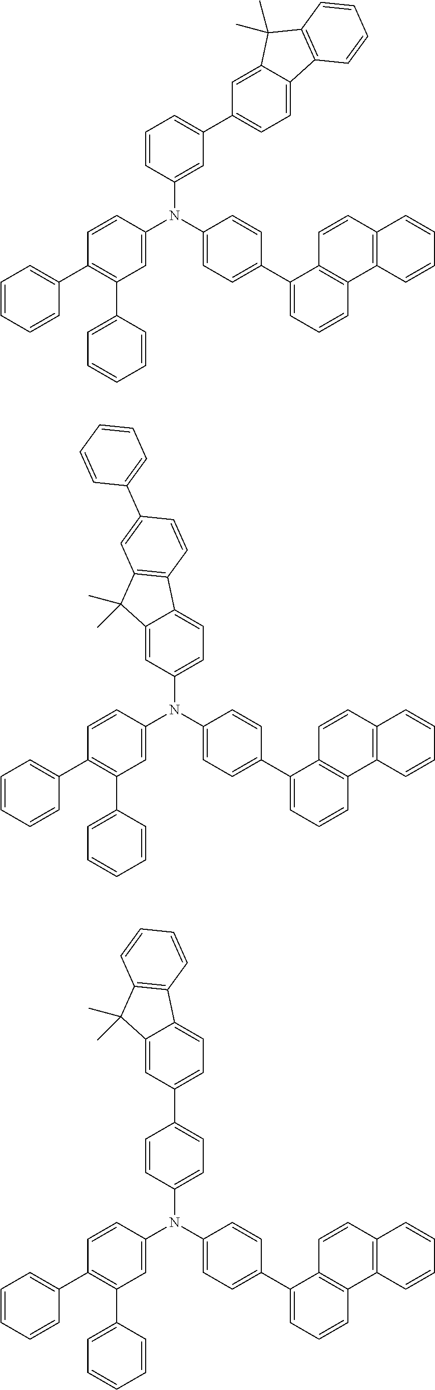

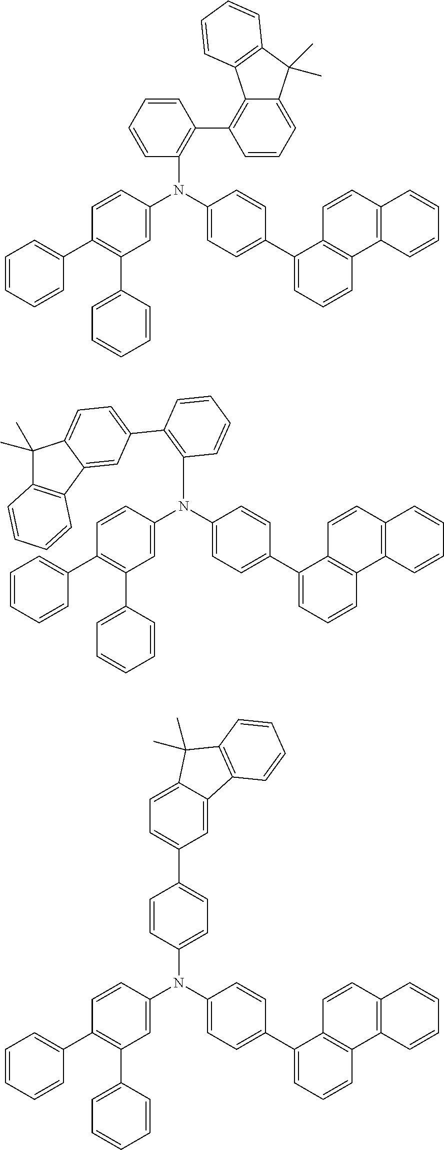 [SMILES: CC1(C)c2ccccc2-c2c(-c3ccccc3N(c3ccc(-c4cccc5c4ccc4ccccc45)cc3)c3ccc(-c4ccccc4)c(-c4ccccc4)c3)cccc21 ; CC1(C)c2ccccc2-c2cc(-c3ccccc3N(c3ccc(-c4cccc5c4ccc4ccccc45)cc3)c3ccc(-c4ccccc4)c(-c4ccccc4)c3)ccc21 ; CC1(C)c2ccccc2-c2cc(-c3ccc(N(c4ccc(-c5cccc6c5ccc5ccccc56)cc4)c4ccc(-c5ccccc5)c(-c5ccccc5)c4)cc3)ccc21]

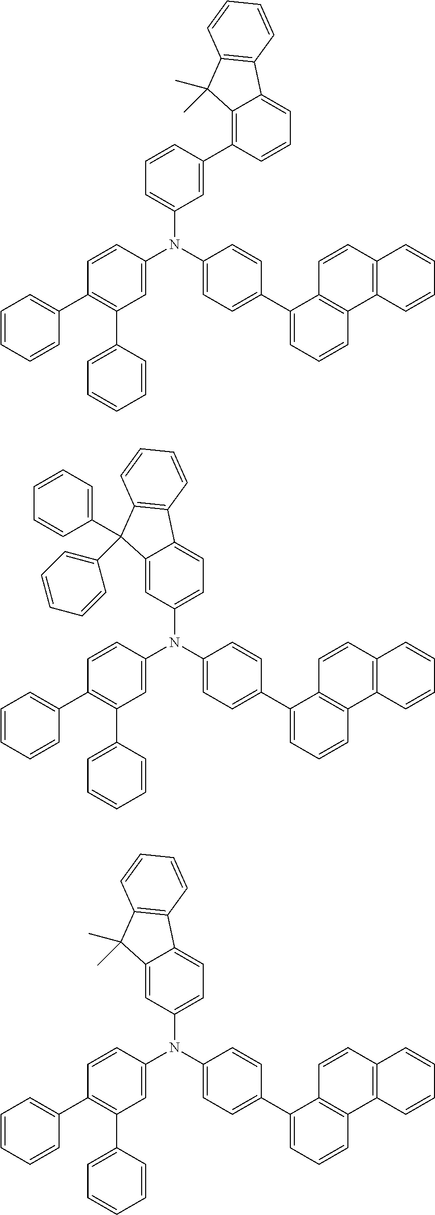

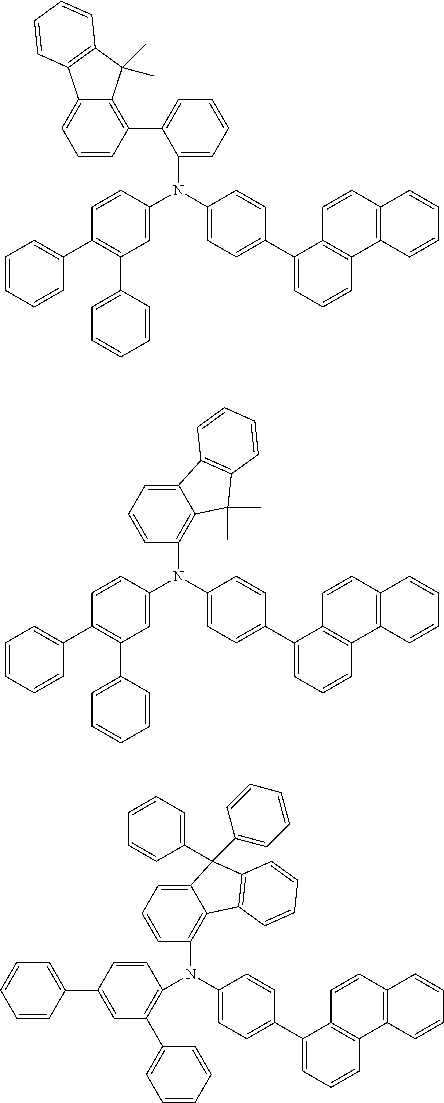 [SMILES: CC1(C)c2ccccc2-c2cccc(-c3ccccc3N(c3ccc(-c4cccc5c4ccc4ccccc45)cc3)c3ccc(-c4ccccc4)c(-c4ccccc4)c3)c21 ; CC1(C)c2ccccc2-c2cccc(N(c3ccc(-c4cccc5c4ccc4ccccc45)cc3)c3ccc(-c4ccccc4)c(-c4ccccc4)c3)c21 ; c1ccc(-c2ccc(N(c3ccc(-c4cccc5c4ccc4ccccc45)cc3)c3cccc4c3-c3ccccc3C4(c3ccccc3)c3ccccc3)c(-c3ccccc3)c2)cc1]

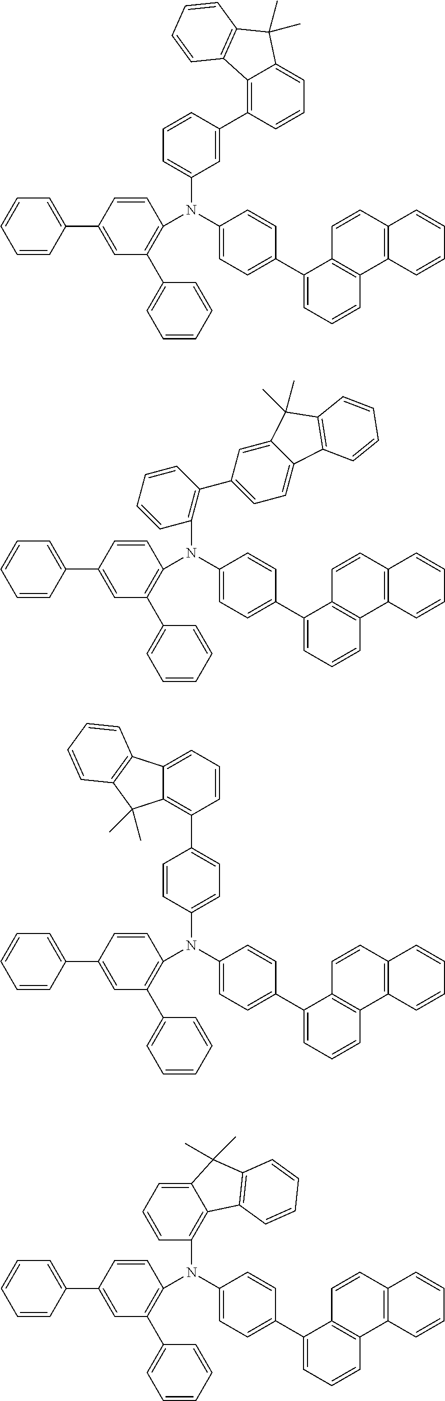 [SMILES: CC1(C)c2ccccc2-c2c(-c3cccc(N(c4ccc(-c5cccc6c5ccc5ccccc56)cc4)c4ccc(-c5ccccc5)cc4-c4ccccc4)c3)cccc21 ; CC1(C)c2ccccc2-c2ccc(-c3ccccc3N(c3ccc(-c4cccc5c4ccc4ccccc45)cc3)c3ccc(-c4ccccc4)cc3-c3ccccc3)cc21 ; CC1(C)c2ccccc2-c2cccc(-c3ccc(N(c4ccc(-c5cccc6c5ccc5ccccc56)cc4)c4ccc(-c5ccccc5)cc4-c4ccccc4)cc3)c21 ; CC1(C)c2ccccc2-c2c(N(c3ccc(-c4cccc5c4ccc4ccccc45)cc3)c3ccc(-c4ccccc4)cc3-c3ccccc3)cccc21]

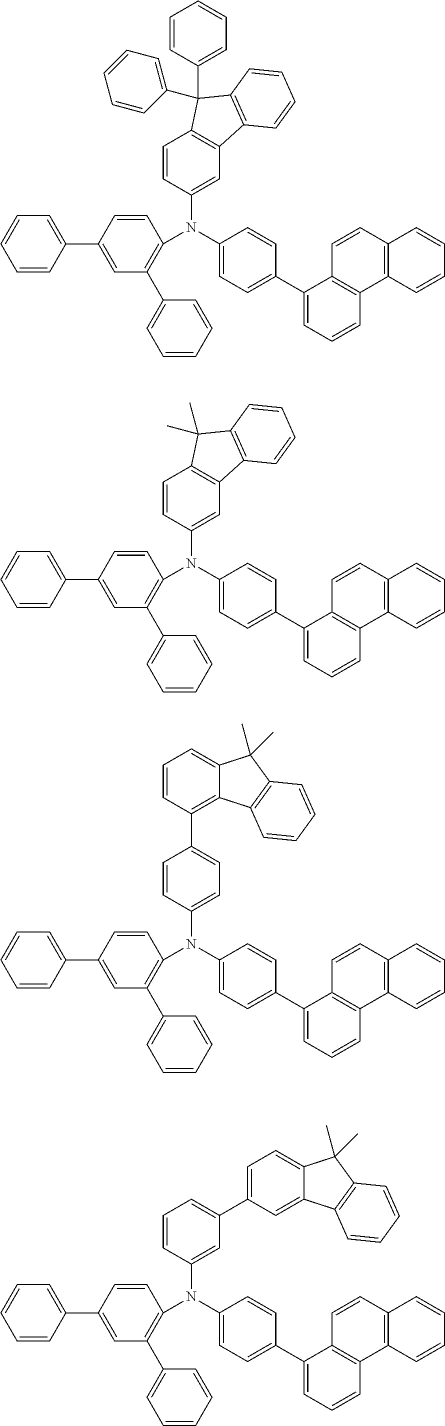 [SMILES: c1ccc(-c2ccc(N(c3ccc(-c4cccc5c4ccc4ccccc45)cc3)c3ccc4c(c3)-c3ccccc3C4(c3ccccc3)c3ccccc3)c(-c3ccccc3)c2)cc1 ; CC1(C)c2ccccc2-c2cc(N(c3ccc(-c4cccc5c4ccc4ccccc45)cc3)c3ccc(-c4ccccc4)cc3-c3ccccc3)ccc21 ; CC1(C)c2ccccc2-c2c(-c3ccc(N(c4ccc(-c5cccc6c5ccc5ccccc56)cc4)c4ccc(-c5ccccc5)cc4-c4ccccc4)cc3)cccc21 ; CC1(C)c2ccccc2-c2cc(-c3cccc(N(c4ccc(-c5cccc6c5ccc5ccccc56)cc4)c4ccc(-c5ccccc5)cc4-c4ccccc4)c3)ccc21]

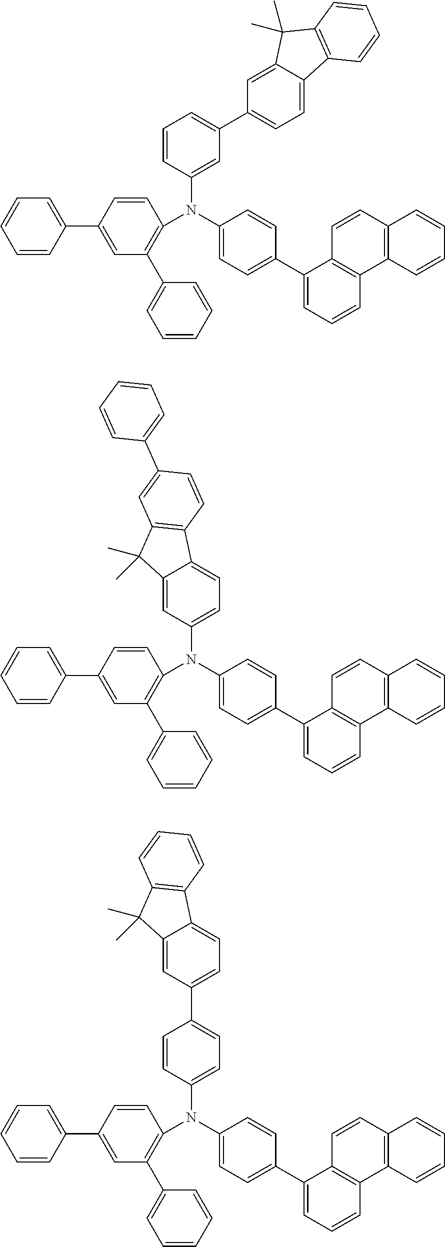

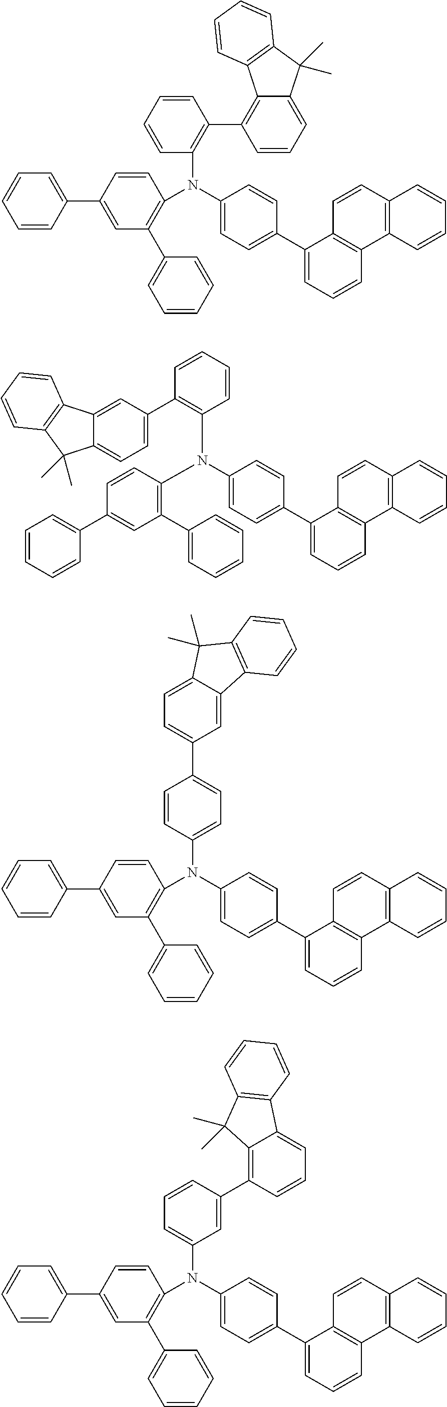

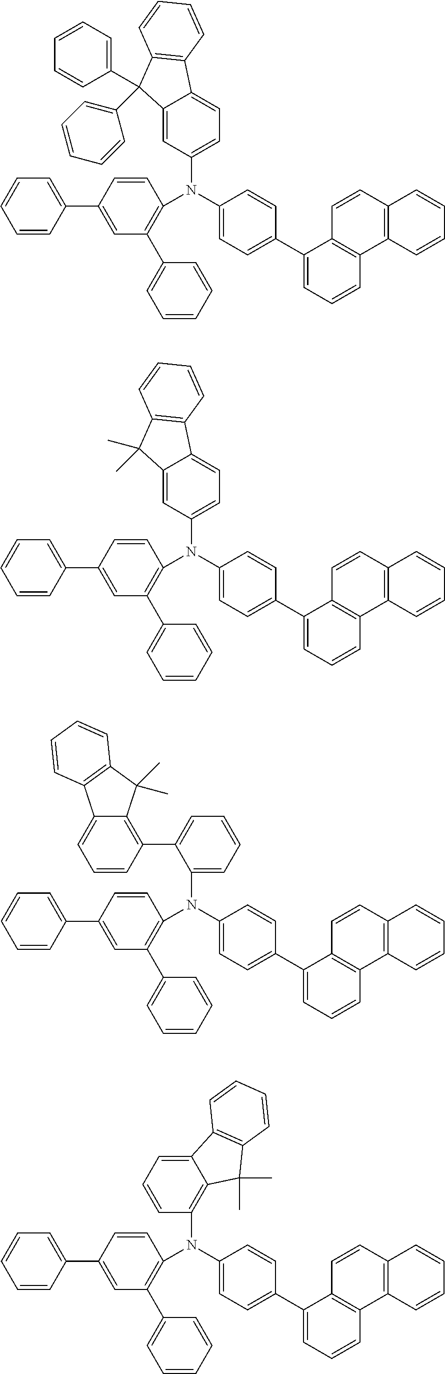

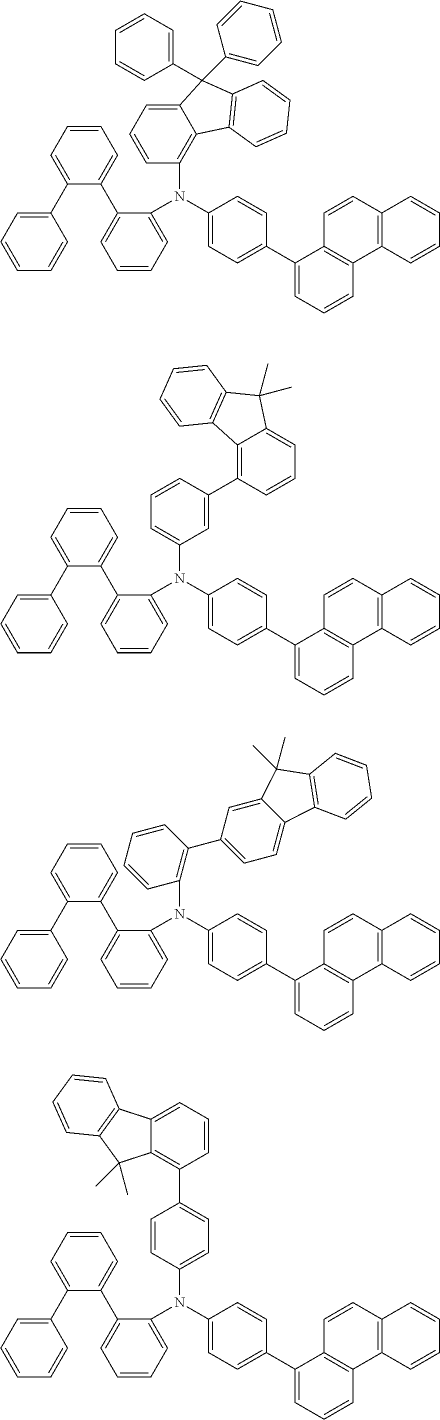 [SMILES: c1ccc(-c2ccccc2-c2ccccc2N(c2ccc(-c3cccc4c3ccc3ccccc34)cc2)c2cccc3c2-c2ccccc2C3(c2ccccc2)c2ccccc2)cc1 ; CC1(C)c2ccccc2-c2c(-c3cccc(N(c4ccc(-c5cccc6c5ccc5ccccc56)cc4)c4ccccc4-c4ccccc4-c4ccccc4)c3)cccc21 ; CC1(C)c2ccccc2-c2ccc(-c3ccccc3N(c3ccc(-c4cccc5c4ccc4ccccc45)cc3)c3ccccc3-c3ccccc3-c3ccccc3)cc21 ; CC1(C)c2ccccc2-c2cccc(-c3ccc(N(c4ccc(-c5cccc6c5ccc5ccccc56)cc4)c4ccccc4-c4ccccc4-c4ccccc4)cc3)c21]

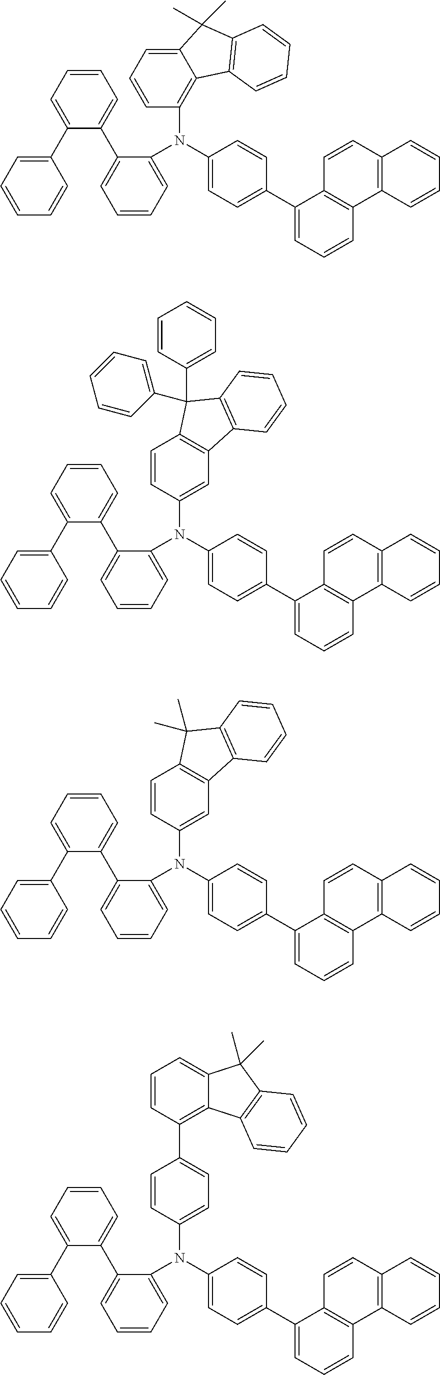 [SMILES: CC1(C)c2ccccc2-c2c(N(c3ccc(-c4cccc5c4ccc4ccccc45)cc3)c3ccccc3-c3ccccc3-c3ccccc3)cccc21 ; c1ccc(-c2ccccc2-c2ccccc2N(c2ccc(-c3cccc4c3ccc3ccccc34)cc2)c2ccc3c(c2)-c2ccccc2C3(c2ccccc2)c2ccccc2)cc1 ; CC1(C)c2ccccc2-c2cc(N(c3ccc(-c4cccc5c4ccc4ccccc45)cc3)c3ccccc3-c3ccccc3-c3ccccc3)ccc21 ; CC1(C)c2ccccc2-c2c(-c3ccc(N(c4ccc(-c5cccc6c5ccc5ccccc56)cc4)c4ccccc4-c4ccccc4-c4ccccc4)cc3)cccc21]

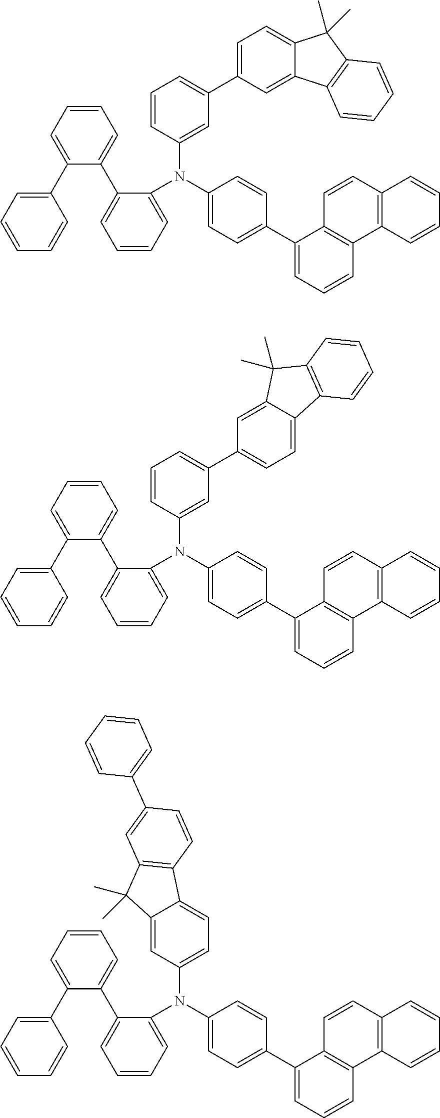

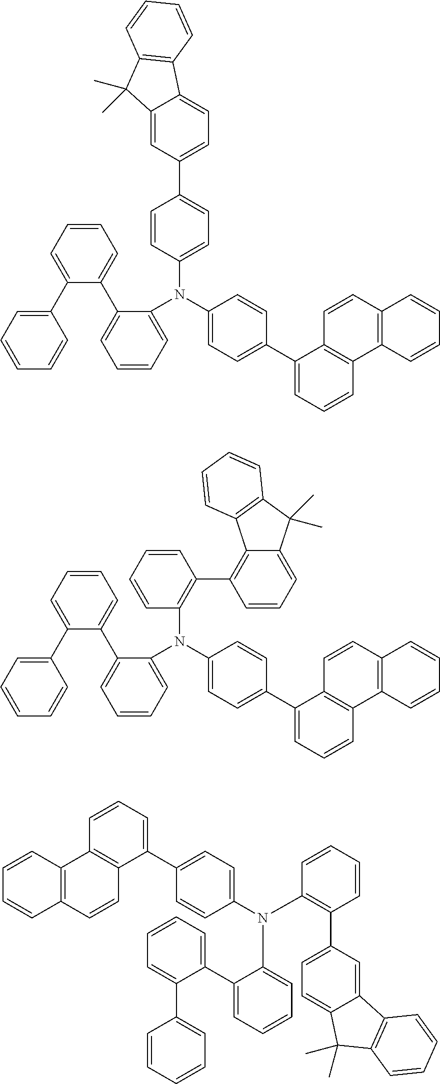 [SMILES: CC1(C)c2ccccc2-c2ccc(-c3ccc(N(c4ccc(-c5cccc6c5ccc5ccccc56)cc4)c4ccccc4-c4ccccc4-c4ccccc4)cc3)cc21 ; CC1(C)c2ccccc2-c2c(-c3ccccc3N(c3ccc(-c4cccc5c4ccc4ccccc45)cc3)c3ccccc3-c3ccccc3-c3ccccc3)cccc21 ; CC1(C)c2ccccc2-c2cc(-c3ccccc3N(c3ccc(-c4cccc5c4ccc4ccccc45)cc3)c3ccccc3-c3ccccc3-c3ccccc3)ccc21]

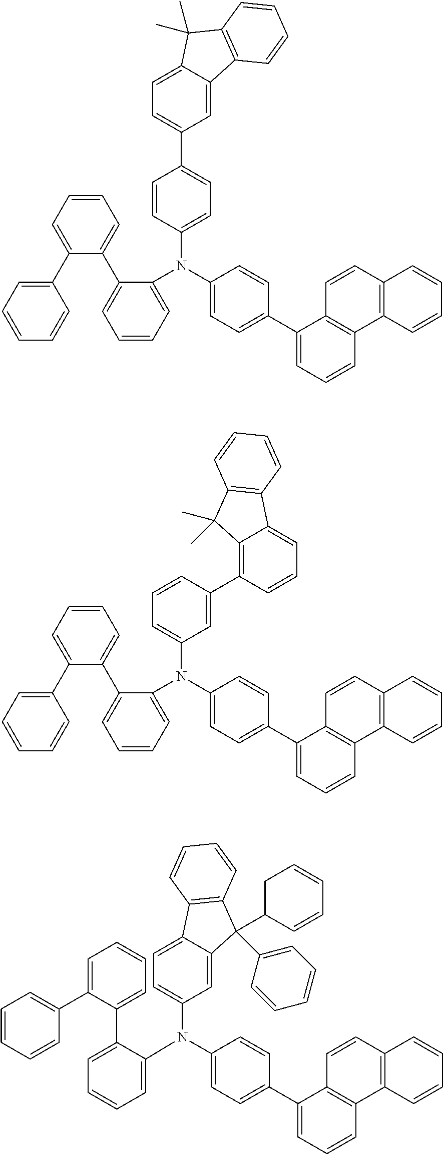 [SMILES: CC1(C)c2ccccc2-c2cc(-c3ccc(N(c4ccc(-c5cccc6c5ccc5ccccc56)cc4)c4ccccc4-c4ccccc4-c4ccccc4)cc3)ccc21 ; CC1(C)c2ccccc2-c2cccc(-c3cccc(N(c4ccc(-c5cccc6c5ccc5ccccc56)cc4)c4ccccc4-c4ccccc4-c4ccccc4)c3)c21 ; C1=CCC(C2(c3ccccc3)c3ccccc3-c3ccc(N(c4ccc(-c5cccc6c5ccc5ccccc56)cc4)c4ccccc4-c4ccccc4-c4ccccc4)cc32)C=C1]

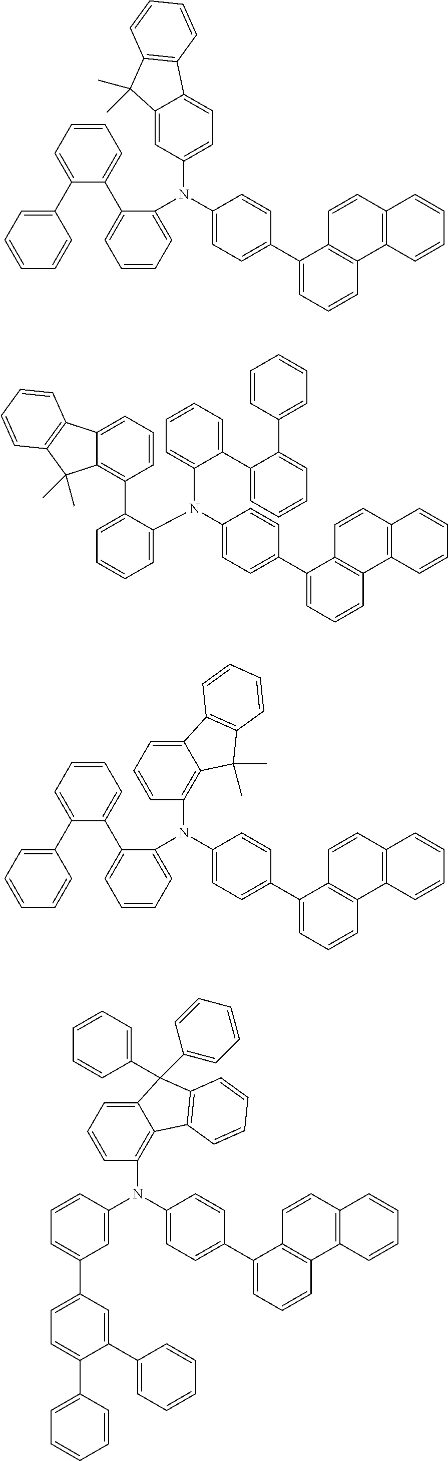 [SMILES: CC1(C)c2ccccc2-c2ccc(N(c3ccc(-c4cccc5c4ccc4ccccc45)cc3)c3ccccc3-c3ccccc3-c3ccccc3)cc21 ; CC1(C)c2ccccc2-c2cccc(-c3ccccc3N(c3ccc(-c4cccc5c4ccc4ccccc45)cc3)c3ccccc3-c3ccccc3-c3ccccc3)c21 ; CC1(C)c2ccccc2-c2cccc(N(c3ccc(-c4cccc5c4ccc4ccccc45)cc3)c3ccccc3-c3ccccc3-c3ccccc3)c21 ; c1ccc(-c2ccc(-c3cccc(N(c4ccc(-c5cccc6c5ccc5ccccc56)cc4)c4cccc5c4-c4ccccc4C5(c4ccccc4)c4ccccc4)c3)cc2-c2ccccc2)cc1]

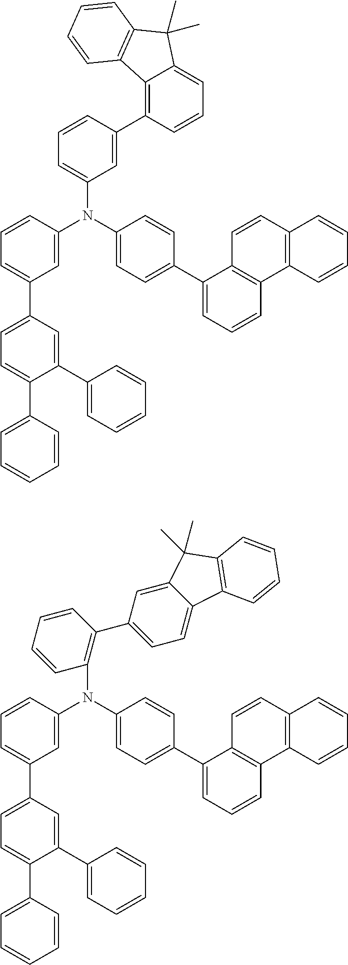 [SMILES: CC1(C)c2ccccc2-c2c(-c3cccc(N(c4ccc(-c5cccc6c5ccc5ccccc56)cc4)c4cccc(-c5ccc(-c6ccccc6)c(-c6ccccc6)c5)c4)c3)cccc21 ; CC1(C)c2ccccc2-c2ccc(-c3ccccc3N(c3ccc(-c4cccc5c4ccc4ccccc45)cc3)c3cccc(-c4ccc(-c5ccccc5)c(-c5ccccc5)c4)c3)cc21]

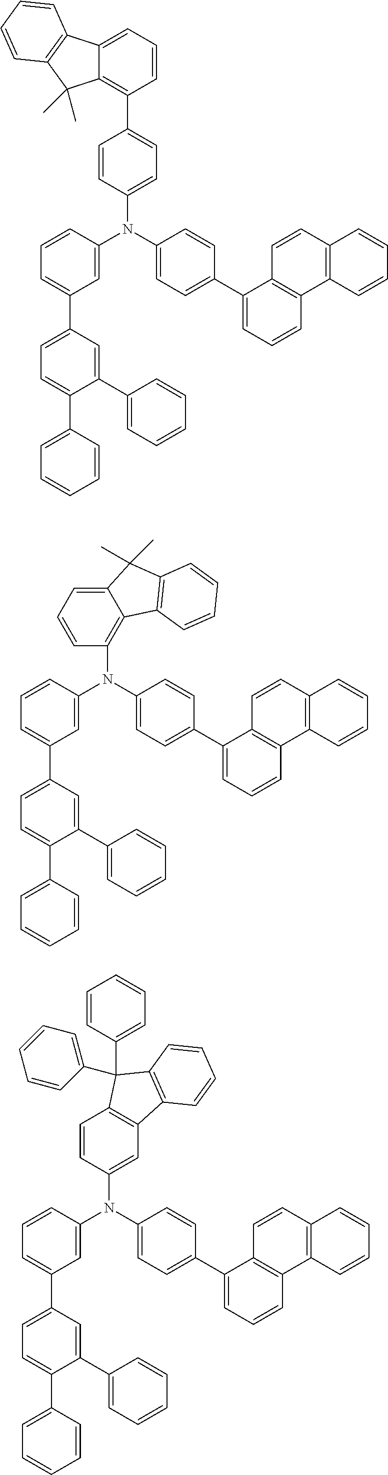 [SMILES: CC1(C)c2ccccc2-c2cccc(-c3ccc(N(c4ccc(-c5cccc6c5ccc5ccccc56)cc4)c4cccc(-c5ccc(-c6ccccc6)c(-c6ccccc6)c5)c4)cc3)c21 ; CC1(C)c2ccccc2-c2c(N(c3ccc(-c4cccc5c4ccc4ccccc45)cc3)c3cccc(-c4ccc(-c5ccccc5)c(-c5ccccc5)c4)c3)cccc21 ; c1ccc(-c2ccc(-c3cccc(N(c4ccc(-c5cccc6c5ccc5ccccc56)cc4)c4ccc5c(c4)-c4ccccc4C5(c4ccccc4)c4ccccc4)c3)cc2-c2ccccc2)cc1]

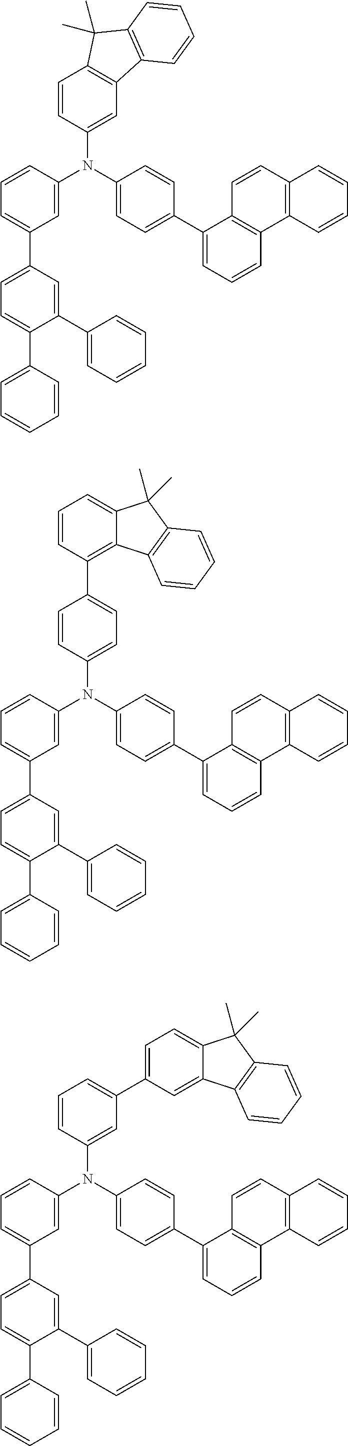 [SMILES: CC1(C)c2ccccc2-c2cc(N(c3ccc(-c4cccc5c4ccc4ccccc45)cc3)c3cccc(-c4ccc(-c5ccccc5)c(-c5ccccc5)c4)c3)ccc21 ; CC1(C)c2ccccc2-c2c(-c3ccc(N(c4ccc(-c5cccc6c5ccc5ccccc56)cc4)c4cccc(-c5ccc(-c6ccccc6)c(-c6ccccc6)c5)c4)cc3)cccc21 ; CC1(C)c2ccccc2-c2cc(-c3cccc(N(c4ccc(-c5cccc6c5ccc5ccccc56)cc4)c4cccc(-c5ccc(-c6ccccc6)c(-c6ccccc6)c5)c4)c3)ccc21]

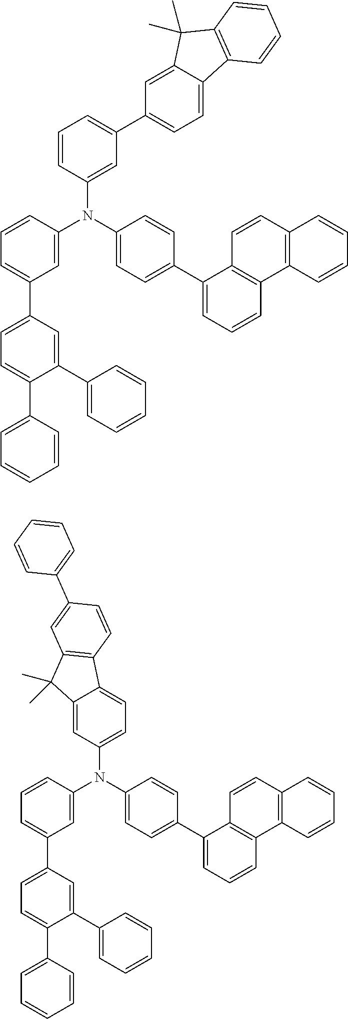

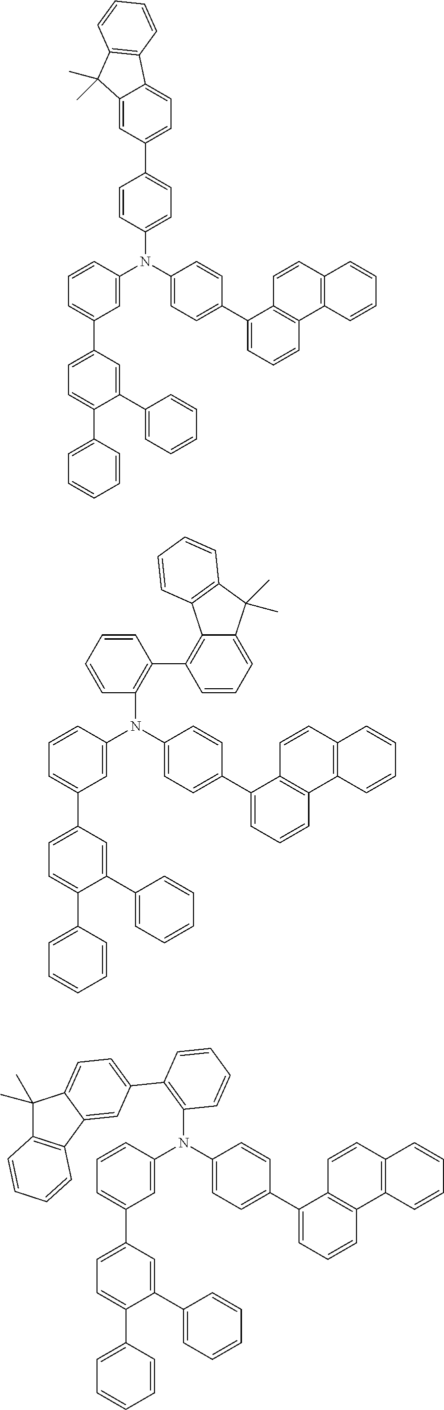 [SMILES: CC1(C)c2ccccc2-c2ccc(-c3ccc(N(c4ccc(-c5cccc6c5ccc5ccccc56)cc4)c4cccc(-c5ccc(-c6ccccc6)c(-c6ccccc6)c5)c4)cc3)cc21 ; CC1(C)c2ccccc2-c2c(-c3ccccc3N(c3ccc(-c4cccc5c4ccc4ccccc45)cc3)c3cccc(-c4ccc(-c5ccccc5)c(-c5ccccc5)c4)c3)cccc21 ; CC1(C)c2ccccc2-c2cc(-c3ccccc3N(c3ccc(-c4cccc5c4ccc4ccccc45)cc3)c3cccc(-c4ccc(-c5ccccc5)c(-c5ccccc5)c4)c3)ccc21]

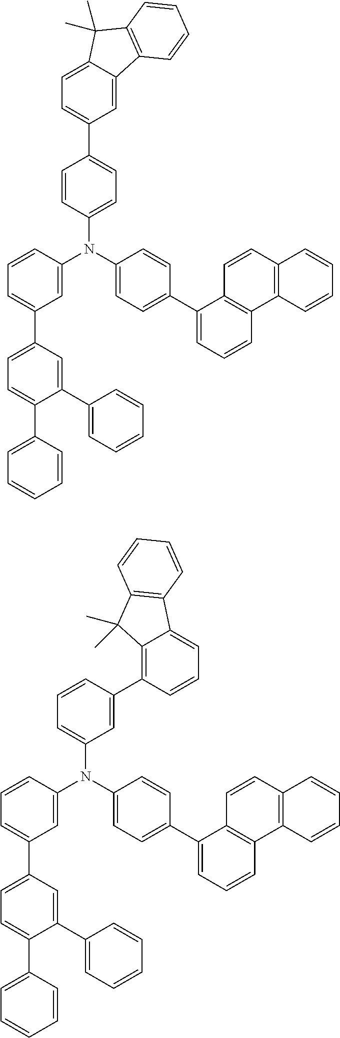 [SMILES: CC1(C)c2ccccc2-c2cc(-c3ccc(N(c4ccc(-c5cccc6c5ccc5ccccc56)cc4)c4cccc(-c5ccc(-c6ccccc6)c(-c6ccccc6)c5)c4)cc3)ccc21 ; CC1(C)c2ccccc2-c2cccc(-c3cccc(N(c4ccc(-c5cccc6c5ccc5ccccc56)cc4)c4cccc(-c5ccc(-c6ccccc6)c(-c6ccccc6)c5)c4)c3)c21]

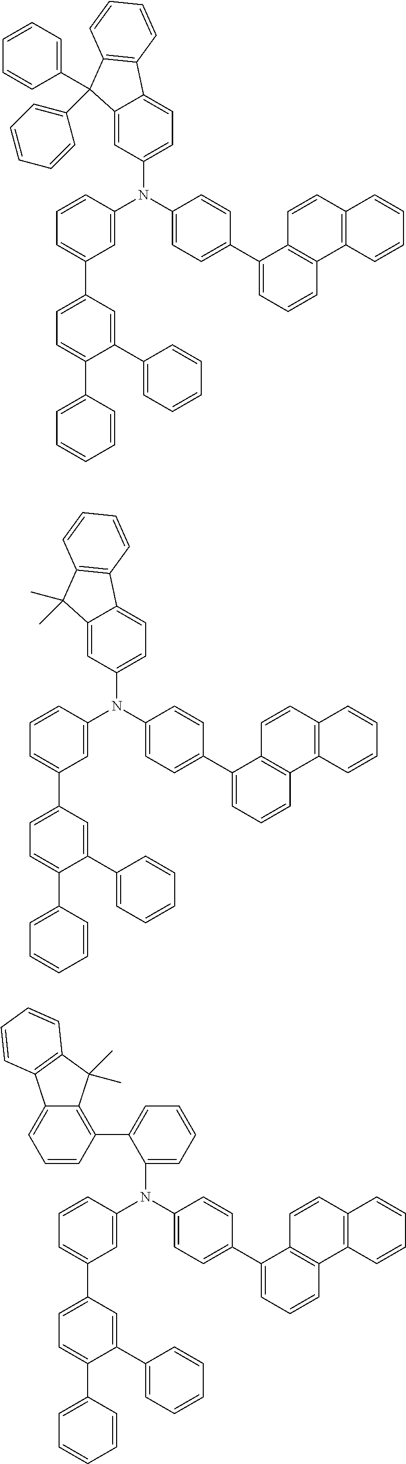

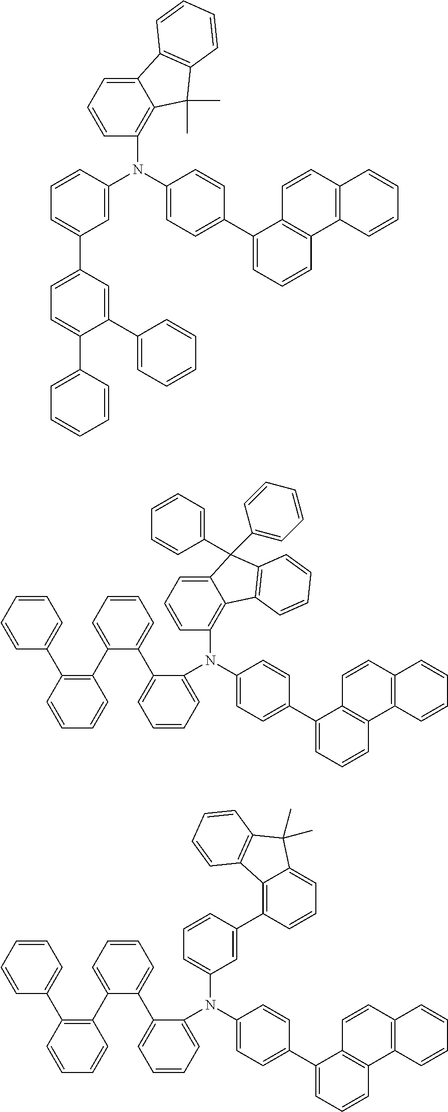 [SMILES: CC1(C)c2ccccc2-c2cccc(N(c3ccc(-c4cccc5c4ccc4ccccc45)cc3)c3cccc(-c4ccc(-c5ccccc5)c(-c5ccccc5)c4)c3)c21 ; c1ccc(-c2ccccc2-c2ccccc2-c2ccccc2N(c2ccc(-c3cccc4c3ccc3ccccc34)cc2)c2cccc3c2-c2ccccc2C3(c2ccccc2)c2ccccc2)cc1 ; CC1(C)c2ccccc2-c2c(-c3cccc(N(c4ccc(-c5cccc6c5ccc5ccccc56)cc4)c4ccccc4-c4ccccc4-c4ccccc4-c4ccccc4)c3)cccc21]

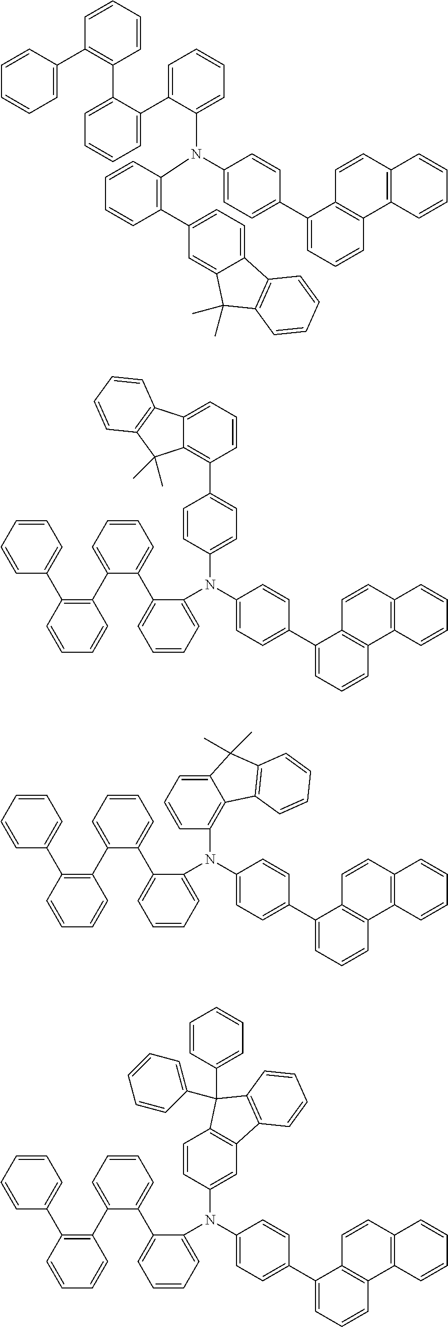 [SMILES: CC1(C)c2ccccc2-c2ccc(-c3ccccc3N(c3ccc(-c4cccc5c4ccc4ccccc45)cc3)c3ccccc3-c3ccccc3-c3ccccc3-c3ccccc3)cc21 ; CC1(C)c2ccccc2-c2cccc(-c3ccc(N(c4ccc(-c5cccc6c5ccc5ccccc56)cc4)c4ccccc4-c4ccccc4-c4ccccc4-c4ccccc4)cc3)c21 ; CC1(C)c2ccccc2-c2c(N(c3ccc(-c4cccc5c4ccc4ccccc45)cc3)c3ccccc3-c3ccccc3-c3ccccc3-c3ccccc3)cccc21 ; c1ccc(-c2ccccc2-c2ccccc2-c2ccccc2N(c2ccc(-c3cccc4c3ccc3ccccc34)cc2)c2ccc3c(c2)-c2ccccc2C3(c2ccccc2)c2ccccc2)cc1]

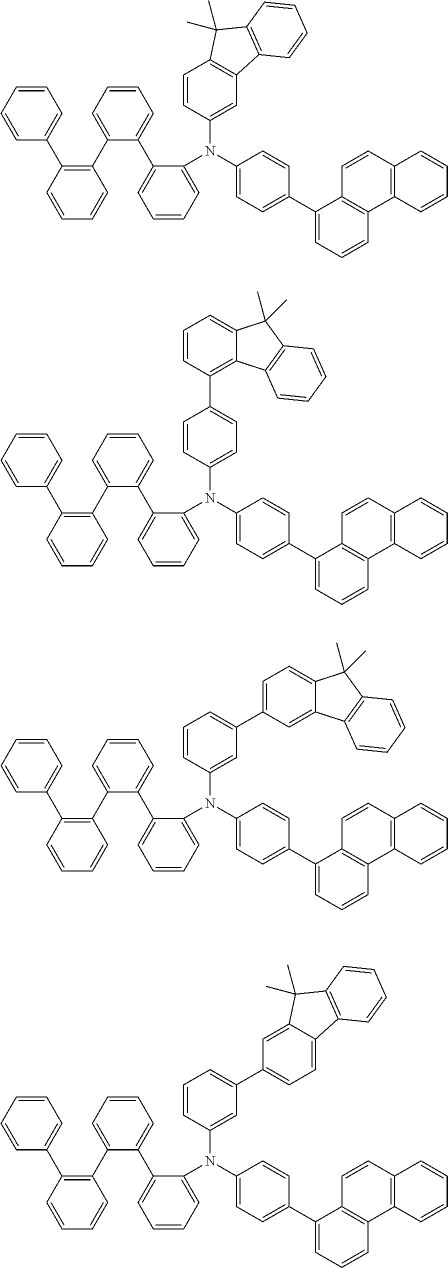

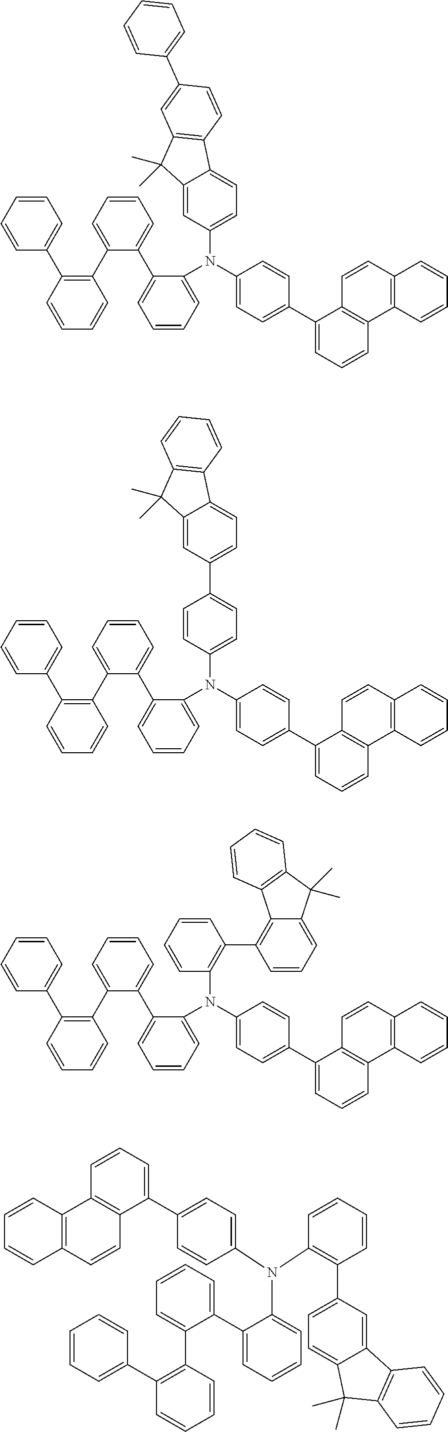 [SMILES: CC1(C)c2cc(-c3ccccc3)ccc2-c2ccc(N(c3ccc(-c4cccc5c4ccc4ccccc45)cc3)c3ccccc3-c3ccccc3-c3ccccc3-c3ccccc3)cc21 ; CC1(C)c2ccccc2-c2ccc(-c3ccc(N(c4ccc(-c5cccc6c5ccc5ccccc56)cc4)c4ccccc4-c4ccccc4-c4ccccc4-c4ccccc4)cc3)cc21 ; CC1(C)c2ccccc2-c2c(-c3ccccc3N(c3ccc(-c4cccc5c4ccc4ccccc45)cc3)c3ccccc3-c3ccccc3-c3ccccc3-c3ccccc3)cccc21 ; CC1(C)c2ccccc2-c2cc(-c3ccccc3N(c3ccc(-c4cccc5c4ccc4ccccc45)cc3)c3ccccc3-c3ccccc3-c3ccccc3-c3ccccc3)ccc21]

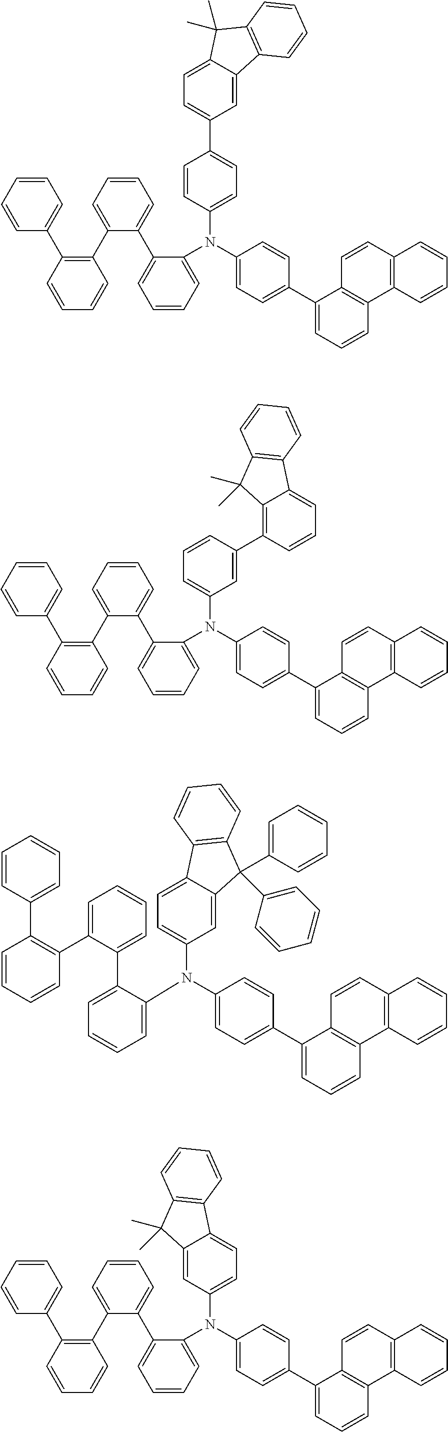 [SMILES: CC1(C)c2ccccc2-c2cc(-c3ccc(N(c4ccc(-c5cccc6c5ccc5ccccc56)cc4)c4ccccc4-c4ccccc4-c4ccccc4-c4ccccc4)cc3)ccc21 ; CC1(C)c2ccccc2-c2cccc(-c3cccc(N(c4ccc(-c5cccc6c5ccc5ccccc56)cc4)c4ccccc4-c4ccccc4-c4ccccc4-c4ccccc4)c3)c21 ; c1ccc(-c2ccccc2-c2ccccc2-c2ccccc2N(c2ccc(-c3cccc4c3ccc3ccccc34)cc2)c2ccc3c(c2)C(c2ccccc2)(c2ccccc2)c2ccccc2-3)cc1 ; CC1(C)c2ccccc2-c2ccc(N(c3ccc(-c4cccc5c4ccc4ccccc45)cc3)c3ccccc3-c3ccccc3-c3ccccc3-c3ccccc3)cc21]

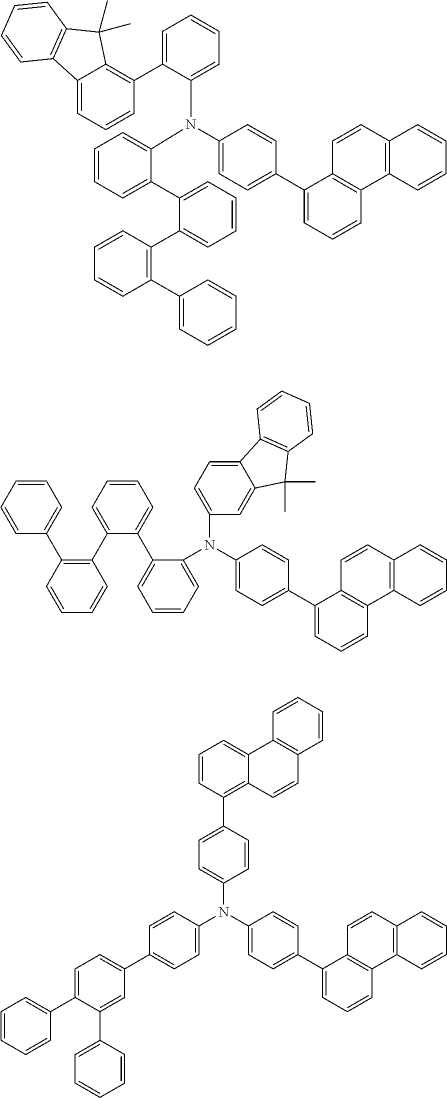 [SMILES: CC1(C)c2ccccc2-c2cccc(-c3ccccc3N(c3ccc(-c4cccc5c4ccc4ccccc45)cc3)c3ccccc3-c3ccccc3-c3ccccc3-c3ccccc3)c21 ; CC1(C)c2ccccc2-c2ccc(N(c3ccc(-c4cccc5c4ccc4ccccc45)cc3)c3ccccc3-c3ccccc3-c3ccccc3-c3ccccc3)cc21 ; c1ccc(-c2ccc(-c3ccc(N(c4ccc(-c5cccc6c5ccc5ccccc56)cc4)c4ccc(-c5cccc6c5ccc5ccccc56)cc4)cc3)cc2-c2ccccc2)cc1]

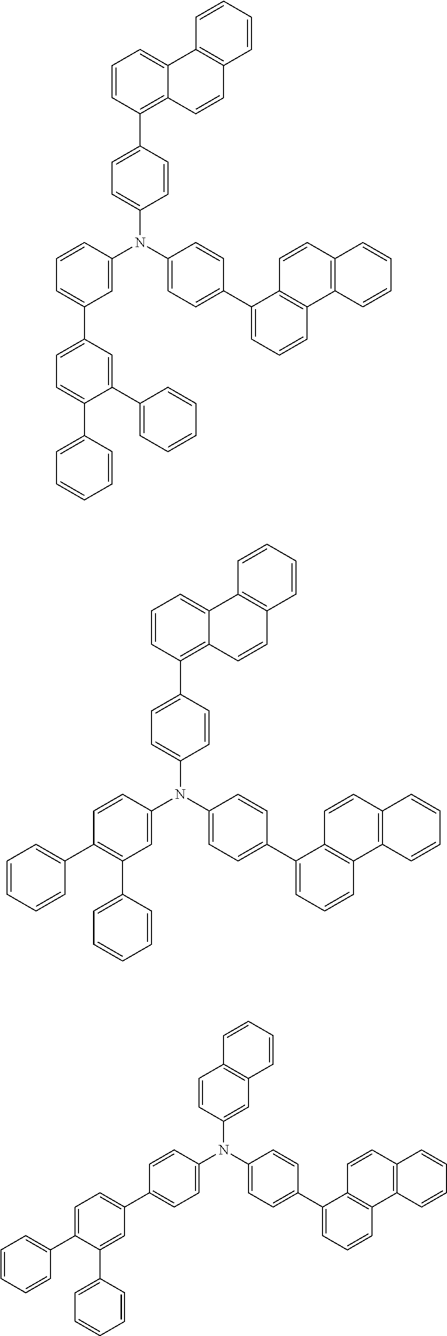 [SMILES: c1ccc(-c2ccc(-c3cccc(N(c4ccc(-c5cccc6c5ccc5ccccc56)cc4)c4ccc(-c5cccc6c5ccc5ccccc56)cc4)c3)cc2-c2ccccc2)cc1 ; c1ccc(-c2ccc(N(c3ccc(-c4cccc5c4ccc4ccccc45)cc3)c3ccc(-c4cccc5c4ccc4ccccc45)cc3)cc2-c2ccccc2)cc1 ; c1ccc(-c2ccc(-c3ccc(N(c4ccc(-c5cccc6c5ccc5ccccc56)cc4)c4ccc5ccccc5c4)cc3)cc2-c2ccccc2)cc1]

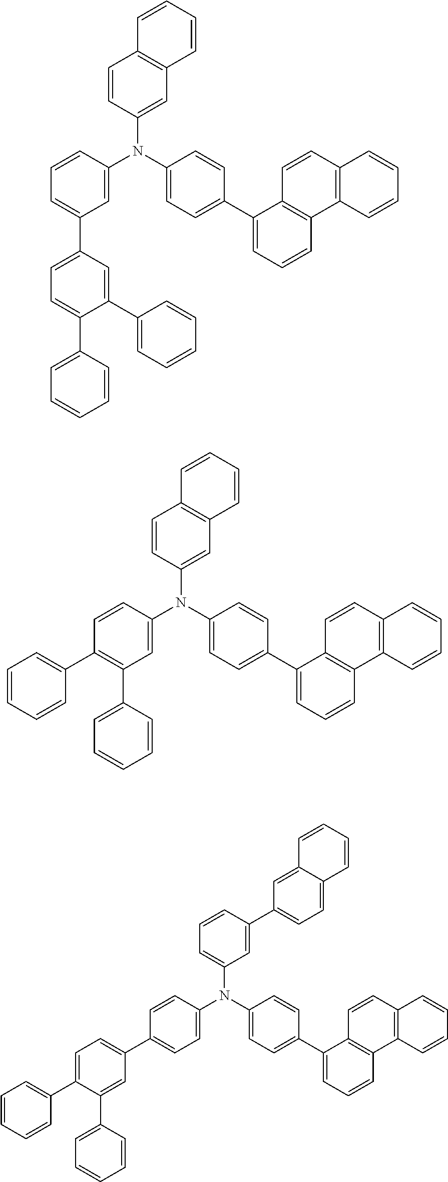 [SMILES: c1ccc(-c2ccc(-c3cccc(N(c4ccc(-c5cccc6c5ccc5ccccc56)cc4)c4ccc5ccccc5c4)c3)cc2-c2ccccc2)cc1 ; c1ccc(-c2ccc(N(c3ccc(-c4cccc5c4ccc4ccccc45)cc3)c3ccc4ccccc4c3)cc2-c2ccccc2)cc1 ; c1ccc(-c2ccc(-c3ccc(N(c4ccc(-c5cccc6c5ccc5ccccc56)cc4)c4cccc(-c5ccc6ccccc6c5)c4)cc3)cc2-c2ccccc2)cc1]

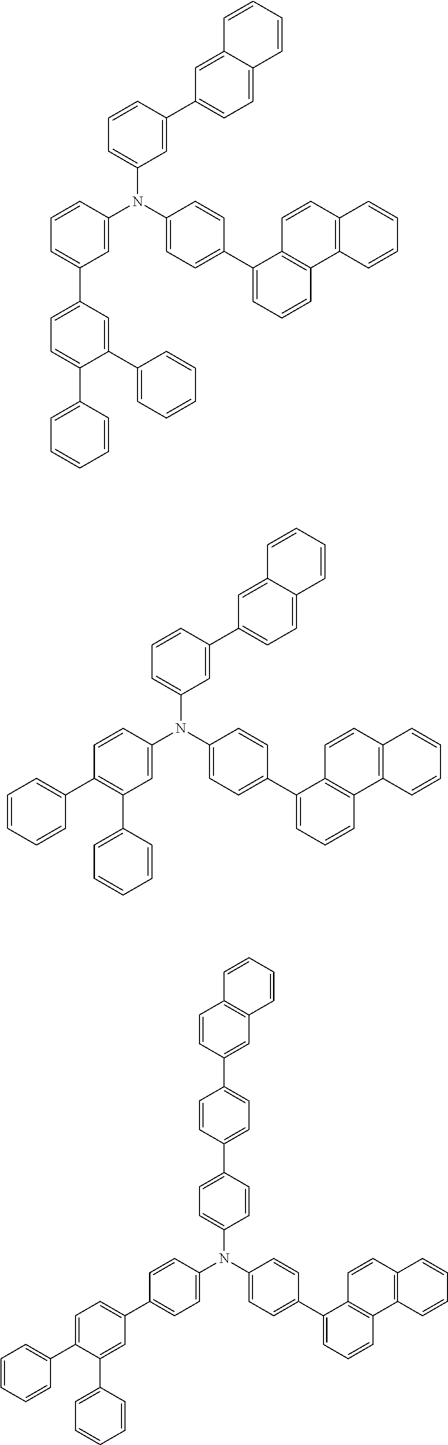 [SMILES: c1ccc(-c2ccc(-c3cccc(N(c4ccc(-c5cccc6c5ccc5ccccc56)cc4)c4cccc(-c5ccc6ccccc6c5)c4)c3)cc2-c2ccccc2)cc1 ; c1ccc(-c2ccc(N(c3ccc(-c4cccc5c4ccc4ccccc45)cc3)c3cccc(-c4ccc5ccccc5c4)c3)cc2-c2ccccc2)cc1 ; c1ccc(-c2ccc(-c3ccc(N(c4ccc(-c5ccc(-c6ccc7ccccc7c6)cc5)cc4)c4ccc(-c5cccc6c5ccc5ccccc56)cc4)cc3)cc2-c2ccccc2)cc1]

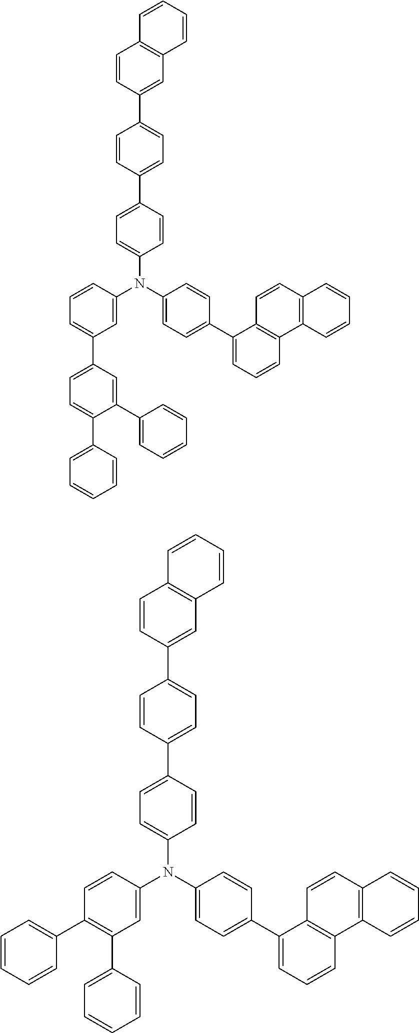 [SMILES: c1ccc(-c2ccc(-c3cccc(N(c4ccc(-c5ccc(-c6ccc7ccccc7c6)cc5)cc4)c4ccc(-c5cccc6c5ccc5ccccc56)cc4)c3)cc2-c2ccccc2)cc1 ; c1ccc(-c2ccc(N(c3ccc(-c4ccc(-c5ccc6ccccc6c5)cc4)cc3)c3ccc(-c4cccc5c4ccc4ccccc45)cc3)cc2-c2ccccc2)cc1]

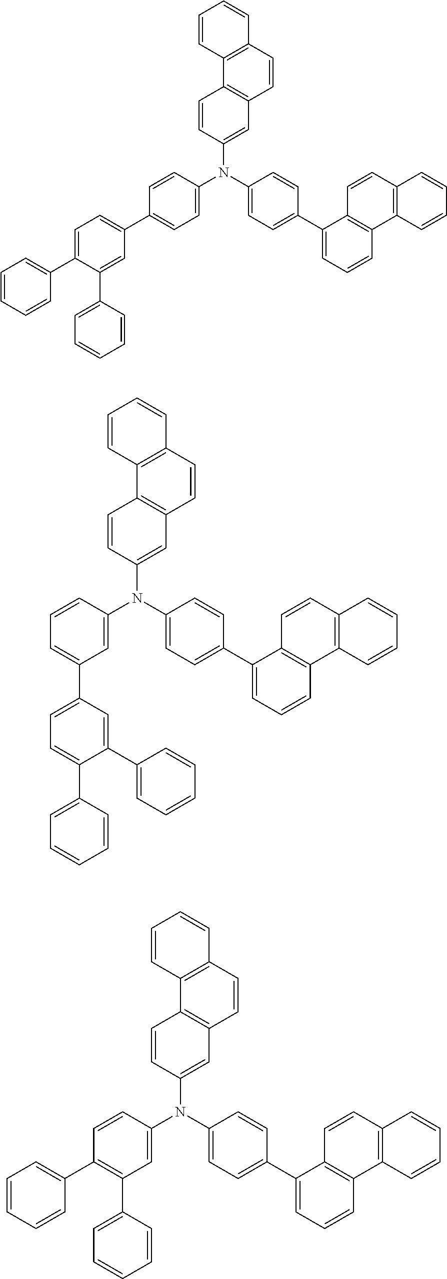 [SMILES: c1ccc(-c2ccc(-c3ccc(N(c4ccc(-c5cccc6c5ccc5ccccc56)cc4)c4ccc5c(ccc6ccccc65)c4)cc3)cc2-c2ccccc2)cc1 ; c1ccc(-c2ccc(-c3cccc(N(c4ccc(-c5cccc6c5ccc5ccccc56)cc4)c4ccc5c(ccc6ccccc65)c4)c3)cc2-c2ccccc2)cc1 ; c1ccc(-c2ccc(N(c3ccc(-c4cccc5c4ccc4ccccc45)cc3)c3ccc4c(ccc5ccccc54)c3)cc2-c2ccccc2)cc1]

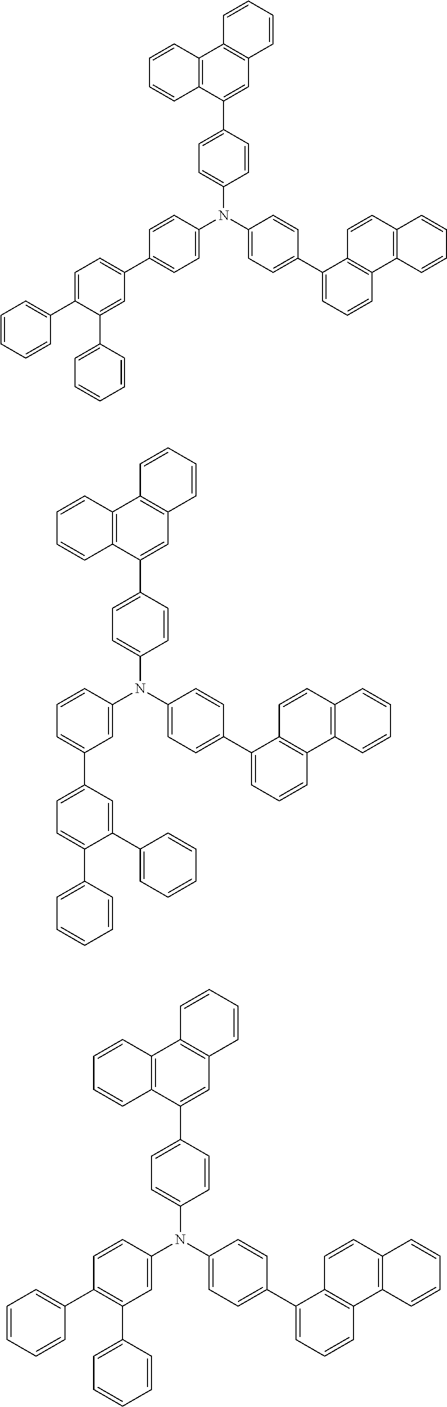 [SMILES: c1ccc(-c2ccc(-c3ccc(N(c4ccc(-c5cccc6c5ccc5ccccc56)cc4)c4ccc(-c5cc6ccccc6c6ccccc56)cc4)cc3)cc2-c2ccccc2)cc1 ; c1ccc(-c2ccc(-c3cccc(N(c4ccc(-c5cccc6c5ccc5ccccc56)cc4)c4ccc(-c5cc6ccccc6c6ccccc56)cc4)c3)cc2-c2ccccc2)cc1 ; c1ccc(-c2ccc(N(c3ccc(-c4cccc5c4ccc4ccccc45)cc3)c3ccc(-c4cc5ccccc5c5ccccc45)cc3)cc2-c2ccccc2)cc1]

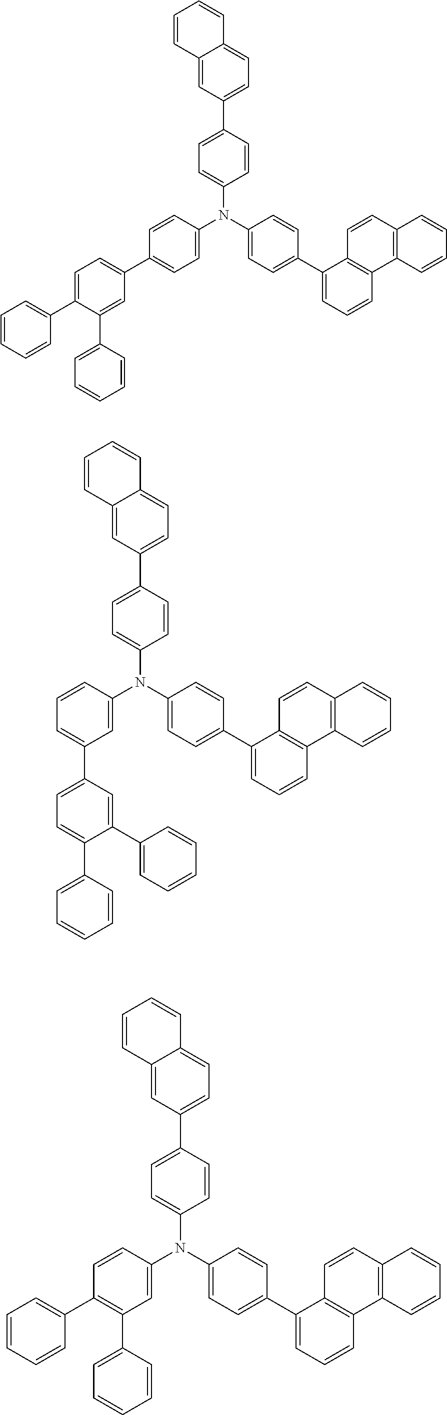

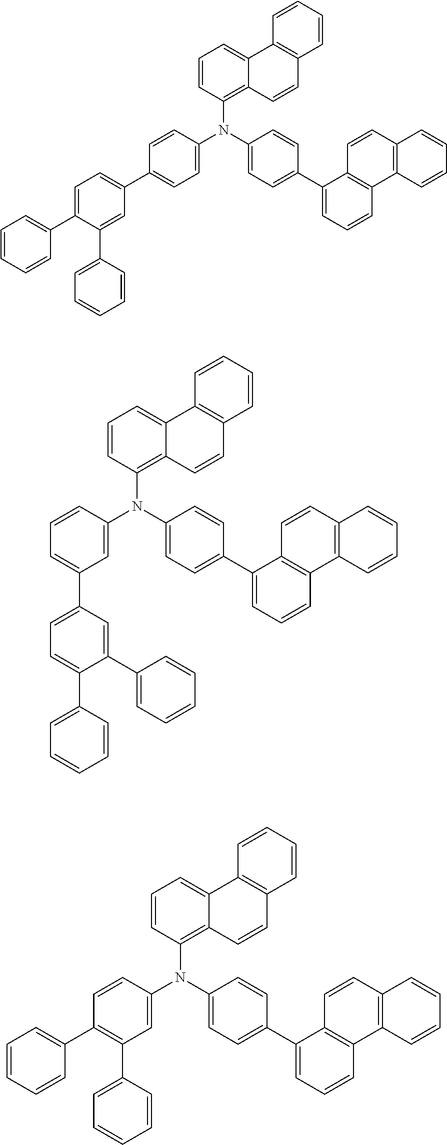 [SMILES: c1ccc(-c2ccc(-c3ccc(N(c4ccc(-c5cccc6c5ccc5ccccc56)cc4)c4cccc5c4ccc4ccccc45)cc3)cc2-c2ccccc2)cc1 ; c1ccc(-c2ccc(-c3cccc(N(c4ccc(-c5cccc6c5ccc5ccccc56)cc4)c4cccc5c4ccc4ccccc45)c3)cc2-c2ccccc2)cc1 ; c1ccc(-c2ccc(N(c3ccc(-c4cccc5c4ccc4ccccc45)cc3)c3cccc4c3ccc3ccccc34)cc2-c2ccccc2)cc1]

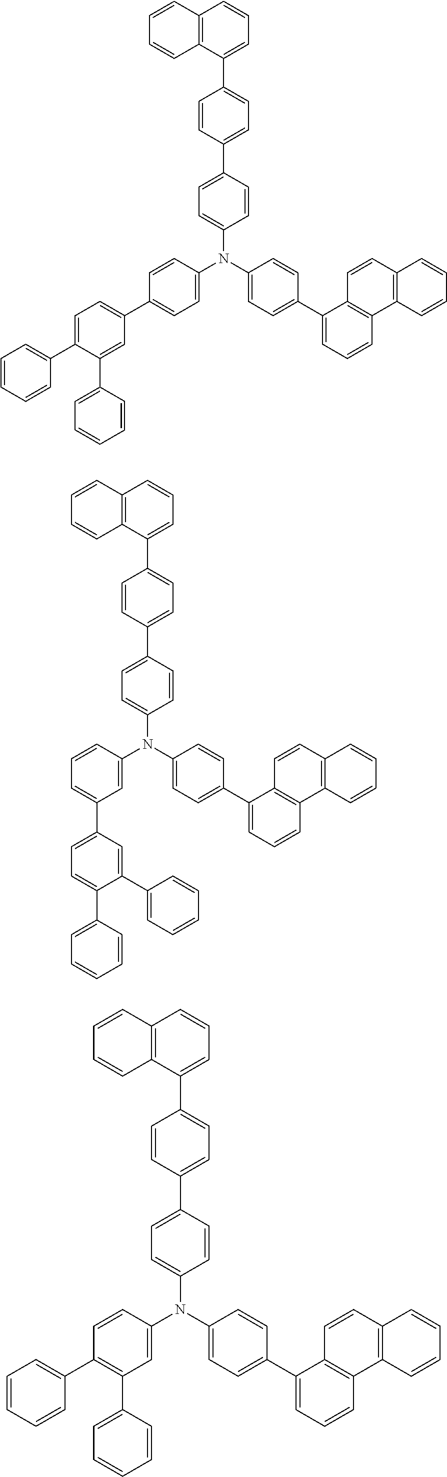

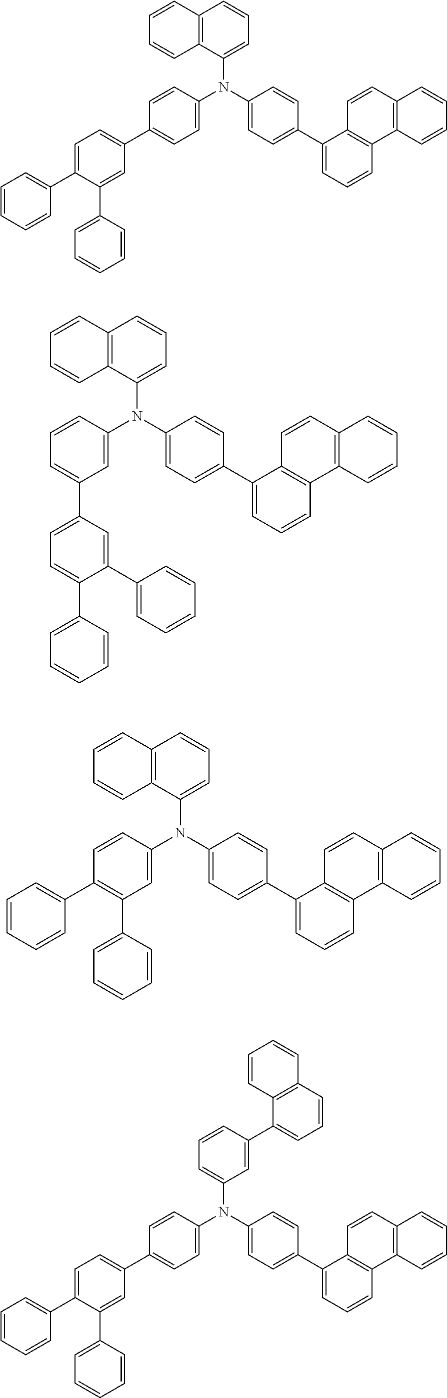 [SMILES: c1ccc(-c2ccc(-c3ccc(N(c4ccc(-c5cccc6c5ccc5ccccc56)cc4)c4cccc5ccccc45)cc3)cc2-c2ccccc2)cc1 ; c1ccc(-c2ccc(-c3cccc(N(c4ccc(-c5cccc6c5ccc5ccccc56)cc4)c4cccc5ccccc45)c3)cc2-c2ccccc2)cc1 ; c1ccc(-c2ccc(N(c3ccc(-c4cccc5c4ccc4ccccc45)cc3)c3cccc4ccccc34)cc2-c2ccccc2)cc1 ; c1ccc(-c2ccc(-c3ccc(N(c4ccc(-c5cccc6c5ccc5ccccc56)cc4)c4cccc(-c5cccc6ccccc56)c4)cc3)cc2-c2ccccc2)cc1]

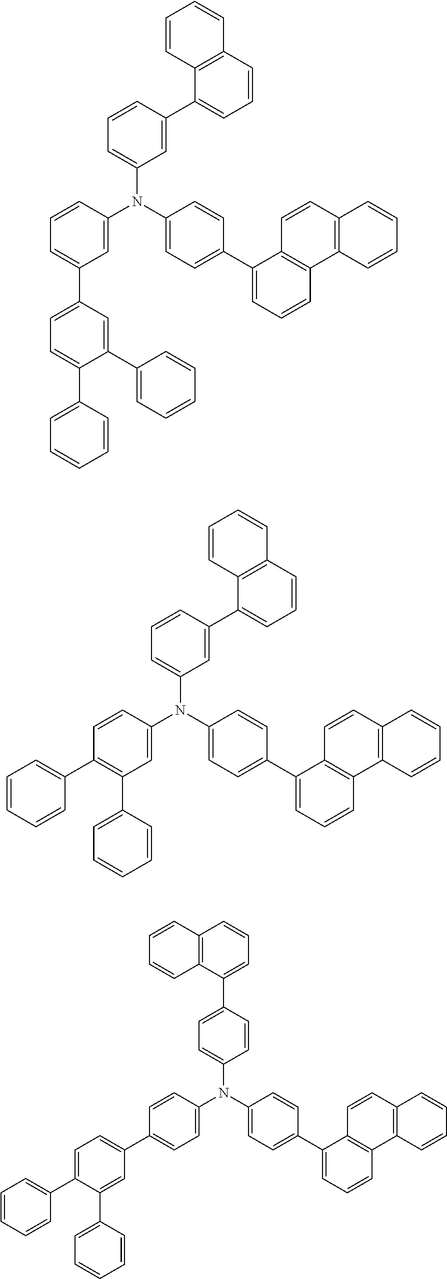 [SMILES: c1ccc(-c2ccc(-c3cccc(N(c4ccc(-c5cccc6c5ccc5ccccc56)cc4)c4cccc(-c5cccc6ccccc56)c4)c3)cc2-c2ccccc2)cc1 ; c1ccc(-c2ccc(N(c3ccc(-c4cccc5c4ccc4ccccc45)cc3)c3cccc(-c4cccc5ccccc45)c3)cc2-c2ccccc2)cc1 ; c1ccc(-c2ccc(-c3ccc(N(c4ccc(-c5cccc6ccccc56)cc4)c4ccc(-c5cccc6c5ccc5ccccc56)cc4)cc3)cc2-c2ccccc2)cc1]

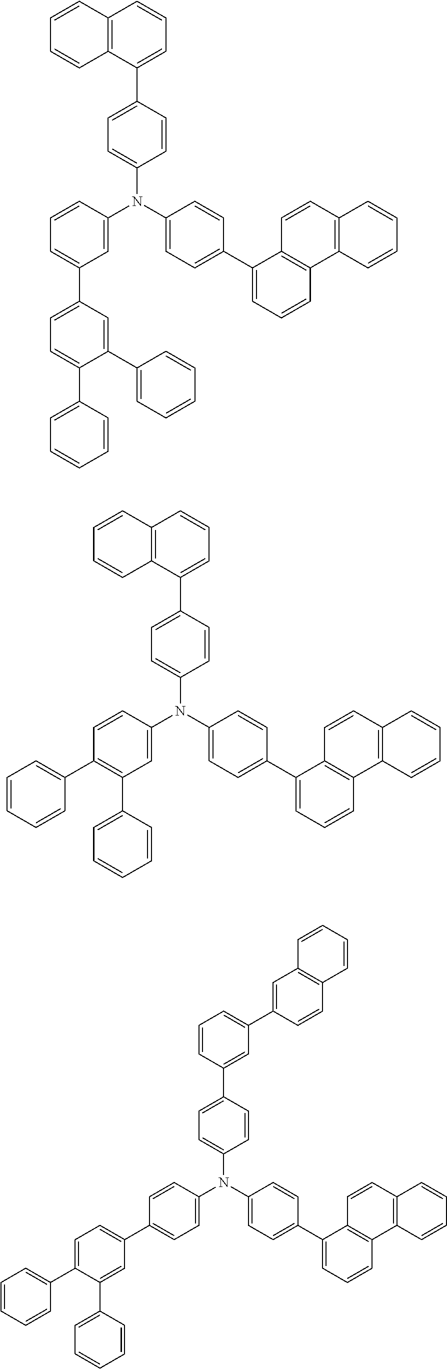 [SMILES: c1ccc(-c2ccc(-c3cccc(N(c4ccc(-c5cccc6ccccc56)cc4)c4ccc(-c5cccc6c5ccc5ccccc56)cc4)c3)cc2-c2ccccc2)cc1 ; c1ccc(-c2ccc(N(c3ccc(-c4cccc5ccccc45)cc3)c3ccc(-c4cccc5c4ccc4ccccc45)cc3)cc2-c2ccccc2)cc1 ; c1ccc(-c2ccc(-c3ccc(N(c4ccc(-c5cccc(-c6ccc7ccccc7c6)c5)cc4)c4ccc(-c5cccc6c5ccc5ccccc56)cc4)cc3)cc2-c2ccccc2)cc1]

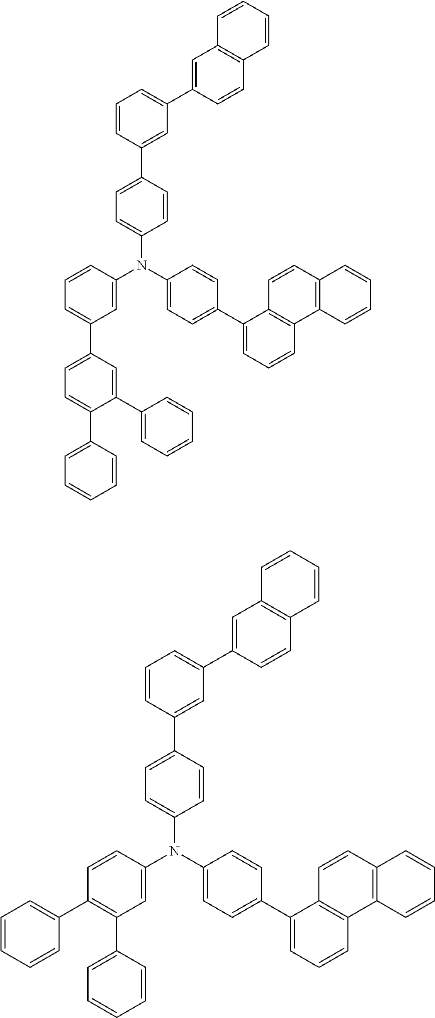 [SMILES: c1ccc(-c2ccc(-c3cccc(N(c4ccc(-c5cccc(-c6ccc7ccccc7c6)c5)cc4)c4ccc(-c5cccc6c5ccc5ccccc56)cc4)c3)cc2-c2ccccc2)cc1 ; c1ccc(-c2ccc(N(c3ccc(-c4cccc(-c5ccc6ccccc6c5)c4)cc3)c3ccc(-c4cccc5c4ccc4ccccc45)cc3)cc2-c2ccccc2)cc1]

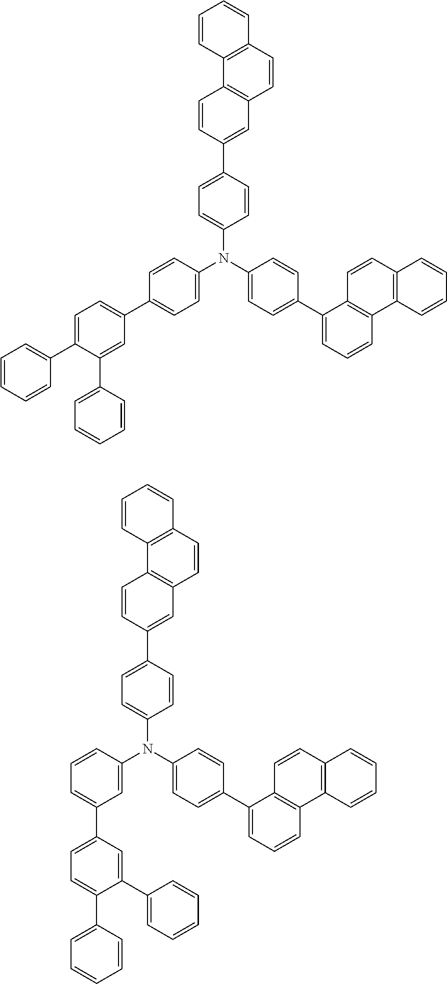 [SMILES: c1ccc(-c2ccc(-c3ccc(N(c4ccc(-c5ccc6c(ccc7ccccc76)c5)cc4)c4ccc(-c5cccc6c5ccc5ccccc56)cc4)cc3)cc2-c2ccccc2)cc1 ; c1ccc(-c2ccc(-c3cccc(N(c4ccc(-c5ccc6c(ccc7ccccc76)c5)cc4)c4ccc(-c5cccc6c5ccc5ccccc56)cc4)c3)cc2-c2ccccc2)cc1]

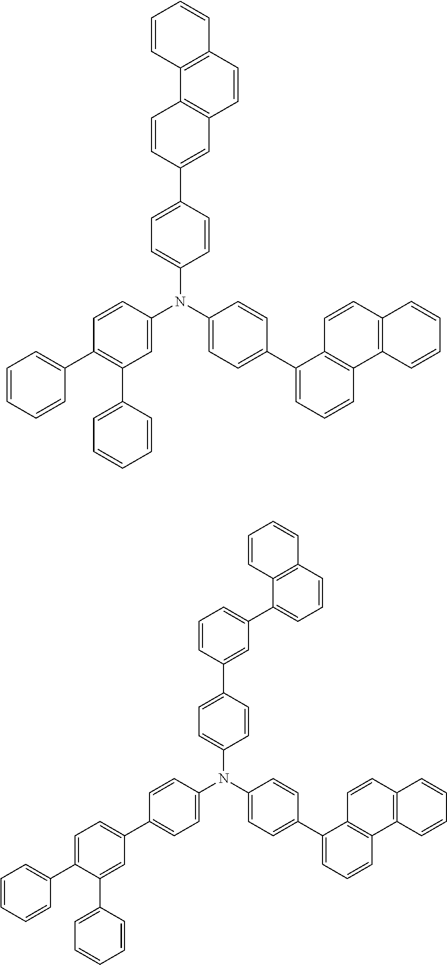 [SMILES: c1ccc(-c2ccc(N(c3ccc(-c4ccc5c(ccc6ccccc65)c4)cc3)c3ccc(-c4cccc5c4ccc4ccccc45)cc3)cc2-c2ccccc2)cc1 ; c1ccc(-c2ccc(-c3ccc(N(c4ccc(-c5cccc(-c6cccc7ccccc67)c5)cc4)c4ccc(-c5cccc6c5ccc5ccccc56)cc4)cc3)cc2-c2ccccc2)cc1]

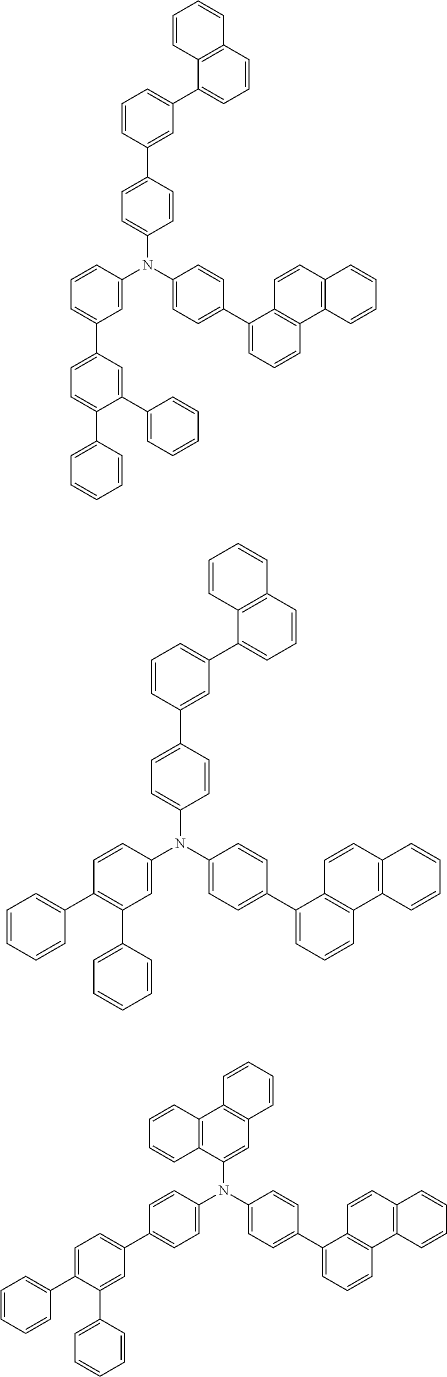 [SMILES: c1ccc(-c2ccc(-c3cccc(N(c4ccc(-c5cccc(-c6cccc7ccccc67)c5)cc4)c4ccc(-c5cccc6c5ccc5ccccc56)cc4)c3)cc2-c2ccccc2)cc1 ; c1ccc(-c2ccc(N(c3ccc(-c4cccc(-c5cccc6ccccc56)c4)cc3)c3ccc(-c4cccc5c4ccc4ccccc45)cc3)cc2-c2ccccc2)cc1 ; c1ccc(-c2ccc(-c3ccc(N(c4ccc(-c5cccc6c5ccc5ccccc56)cc4)c4cc5ccccc5c5ccccc45)cc3)cc2-c2ccccc2)cc1]

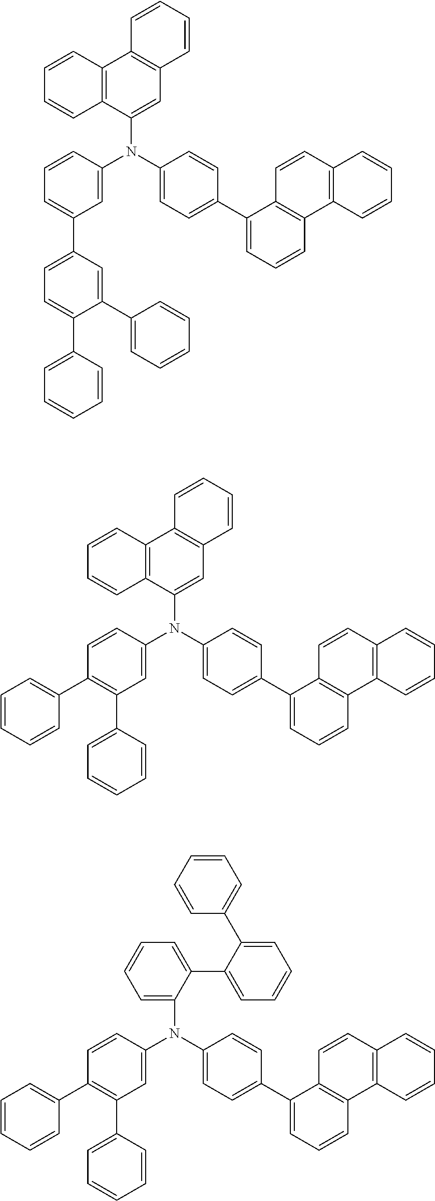 [SMILES: c1ccc(-c2ccc(-c3cccc(N(c4ccc(-c5cccc6c5ccc5ccccc56)cc4)c4cc5ccccc5c5ccccc45)c3)cc2-c2ccccc2)cc1 ; c1ccc(-c2ccc(N(c3ccc(-c4cccc5c4ccc4ccccc45)cc3)c3cc4ccccc4c4ccccc34)cc2-c2ccccc2)cc1 ; c1ccc(-c2ccc(N(c3ccc(-c4cccc5c4ccc4ccccc45)cc3)c3ccccc3-c3ccccc3-c3ccccc3)cc2-c2ccccc2)cc1]

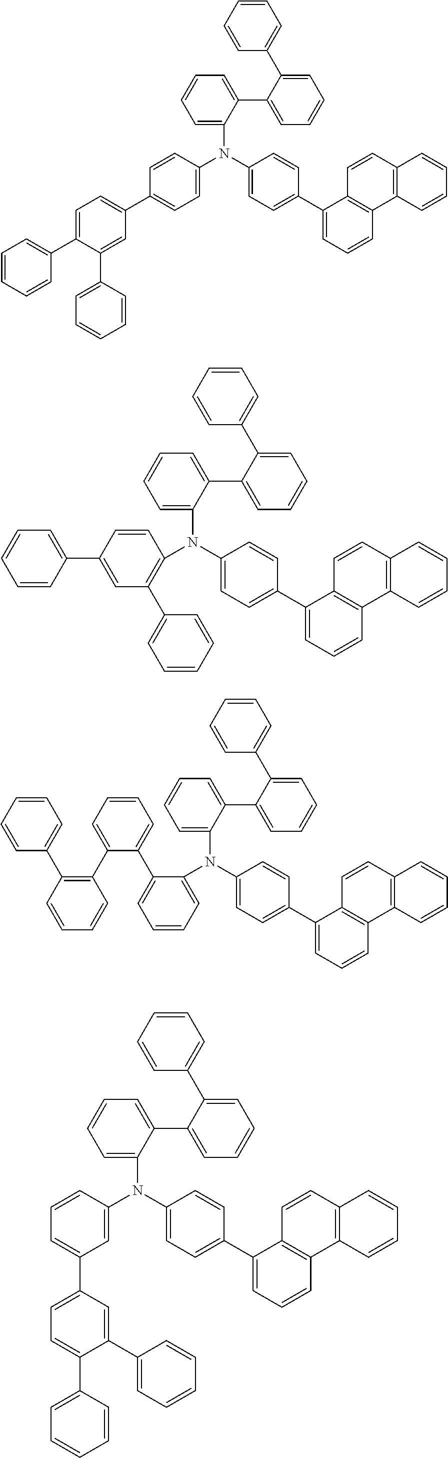

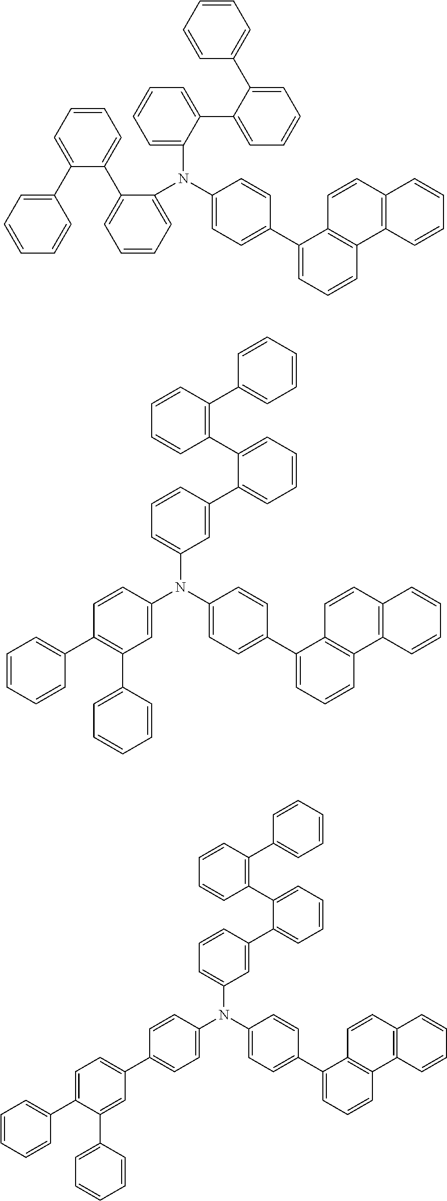 [SMILES: c1ccc(-c2ccccc2-c2ccccc2N(c2ccc(-c3cccc4c3ccc3ccccc34)cc2)c2ccccc2-c2ccccc2-c2ccccc2)cc1 ; c1ccc(-c2ccc(N(c3ccc(-c4cccc5c4ccc4ccccc45)cc3)c3cccc(-c4ccccc4-c4ccccc4-c4ccccc4)c3)cc2-c2ccccc2)cc1 ; c1ccc(-c2ccc(-c3ccc(N(c4ccc(-c5cccc6c5ccc5ccccc56)cc4)c4cccc(-c5ccccc5-c5ccccc5-c5ccccc5)c4)cc3)cc2-c2ccccc2)cc1]

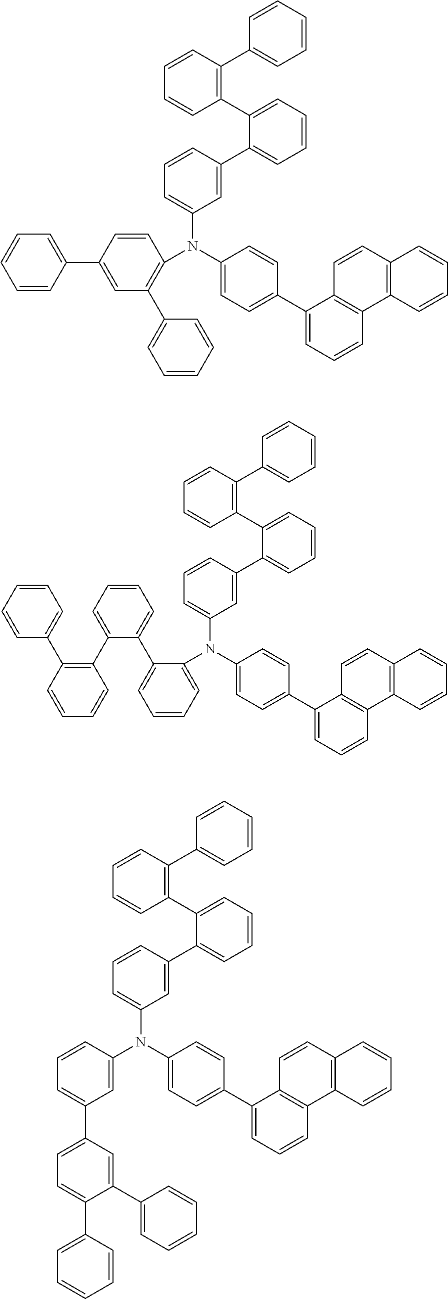 [SMILES: c1ccc(-c2ccc(N(c3ccc(-c4cccc5c4ccc4ccccc45)cc3)c3cccc(-c4ccccc4-c4ccccc4-c4ccccc4)c3)c(-c3ccccc3)c2)cc1 ; c1ccc(-c2ccccc2-c2ccccc2-c2cccc(N(c3ccc(-c4cccc5c4ccc4ccccc45)cc3)c3ccccc3-c3ccccc3-c3ccccc3-c3ccccc3)c2)cc1 ; c1ccc(-c2ccc(-c3cccc(N(c4ccc(-c5cccc6c5ccc5ccccc56)cc4)c4cccc(-c5ccccc5-c5ccccc5-c5ccccc5)c4)c3)cc2-c2ccccc2)cc1]

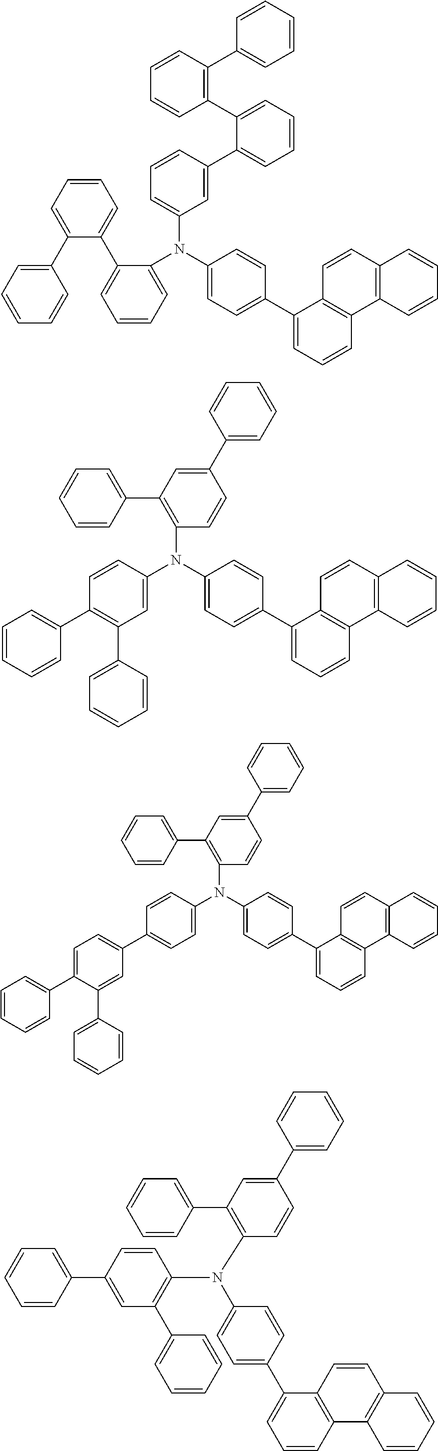 [SMILES: c1ccc(-c2ccccc2-c2ccccc2-c2cccc(N(c3ccc(-c4cccc5c4ccc4ccccc45)cc3)c3ccccc3-c3ccccc3-c3ccccc3)c2)cc1 ; c1ccc(-c2ccc(N(c3ccc(-c4cccc5c4ccc4ccccc45)cc3)c3ccc(-c4ccccc4)c(-c4ccccc4)c3)c(-c3ccccc3)c2)cc1 ; c1ccc(-c2ccc(N(c3ccc(-c4ccc(-c5ccccc5)c(-c5ccccc5)c4)cc3)c3ccc(-c4cccc5c4ccc4ccccc45)cc3)c(-c3ccccc3)c2)cc1 ; c1ccc(-c2ccc(N(c3ccc(-c4cccc5c4ccc4ccccc45)cc3)c3ccc(-c4ccccc4)cc3-c3ccccc3)c(-c3ccccc3)c2)cc1]

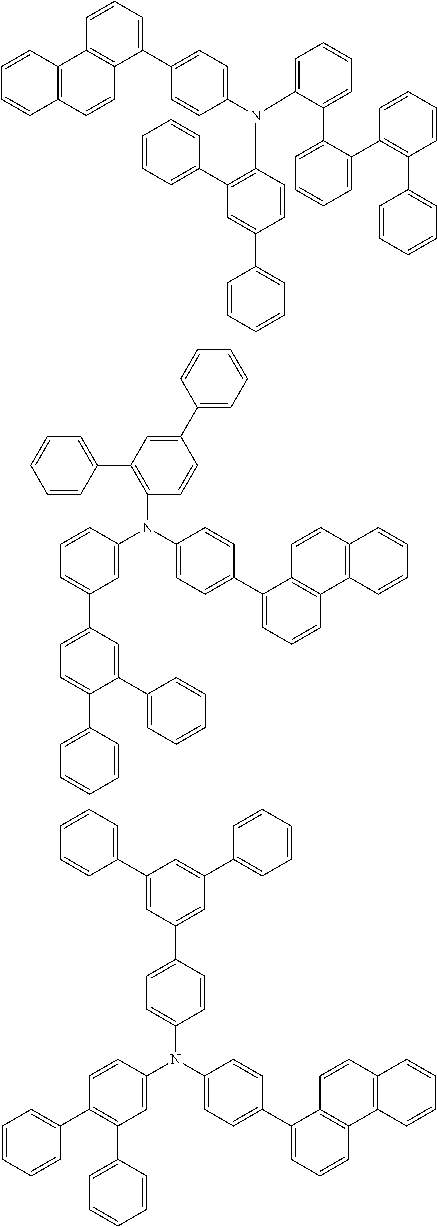

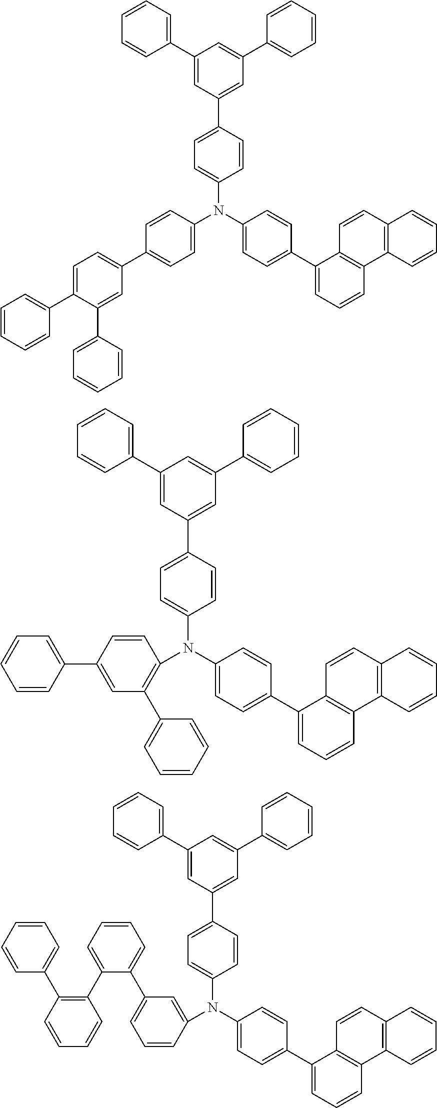

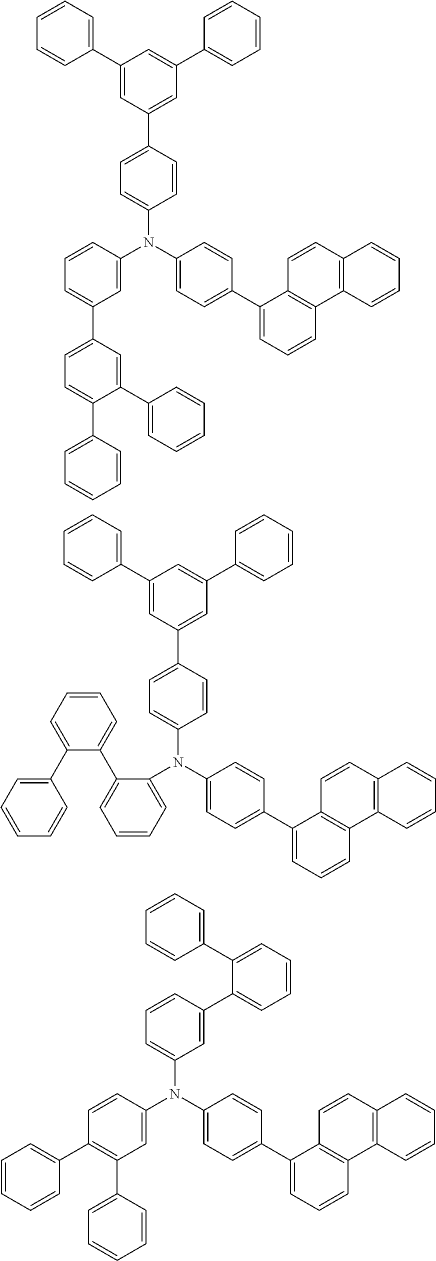

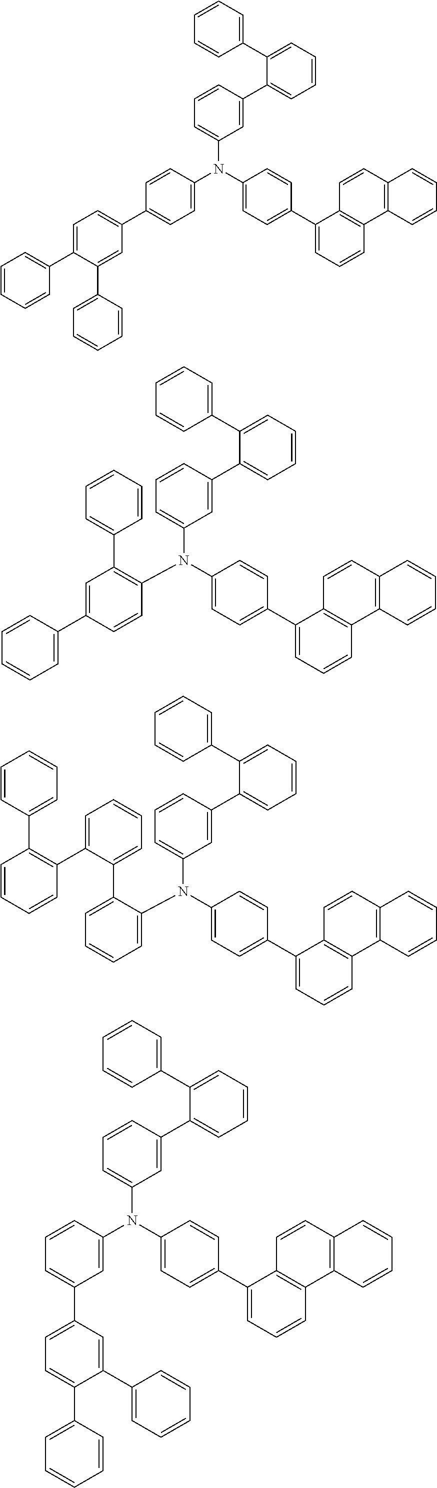 [SMILES: c1ccc(-c2ccccc2-c2cccc(N(c3ccc(-c4ccc(-c5ccccc5)c(-c5ccccc5)c4)cc3)c3ccc(-c4cccc5c4ccc4ccccc45)cc3)c2)cc1 ; c1ccc(-c2ccc(N(c3ccc(-c4cccc5c4ccc4ccccc45)cc3)c3cccc(-c4ccccc4-c4ccccc4)c3)c(-c3ccccc3)c2)cc1 ; c1ccc(-c2ccccc2-c2cccc(N(c3ccc(-c4cccc5c4ccc4ccccc45)cc3)c3ccccc3-c3ccccc3-c3ccccc3-c3ccccc3)c2)cc1 ; c1ccc(-c2ccccc2-c2cccc(N(c3ccc(-c4cccc5c4ccc4ccccc45)cc3)c3cccc(-c4ccc(-c5ccccc5)c(-c5ccccc5)c4)c3)c2)cc1]

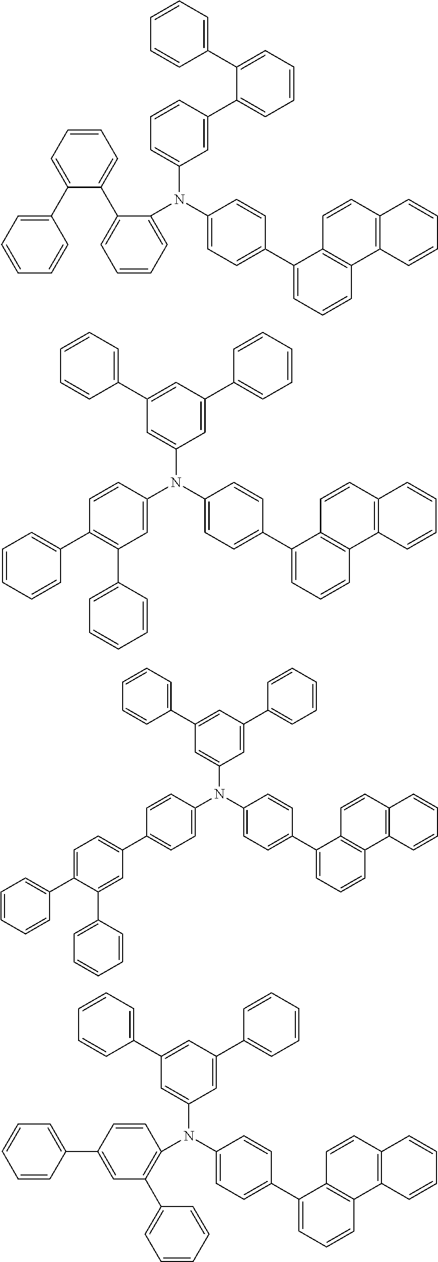

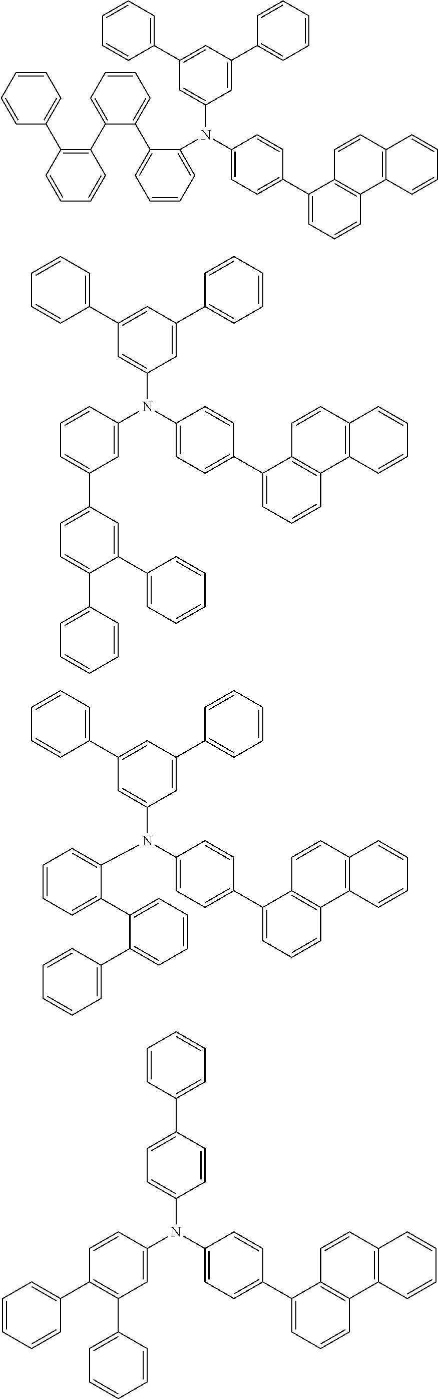

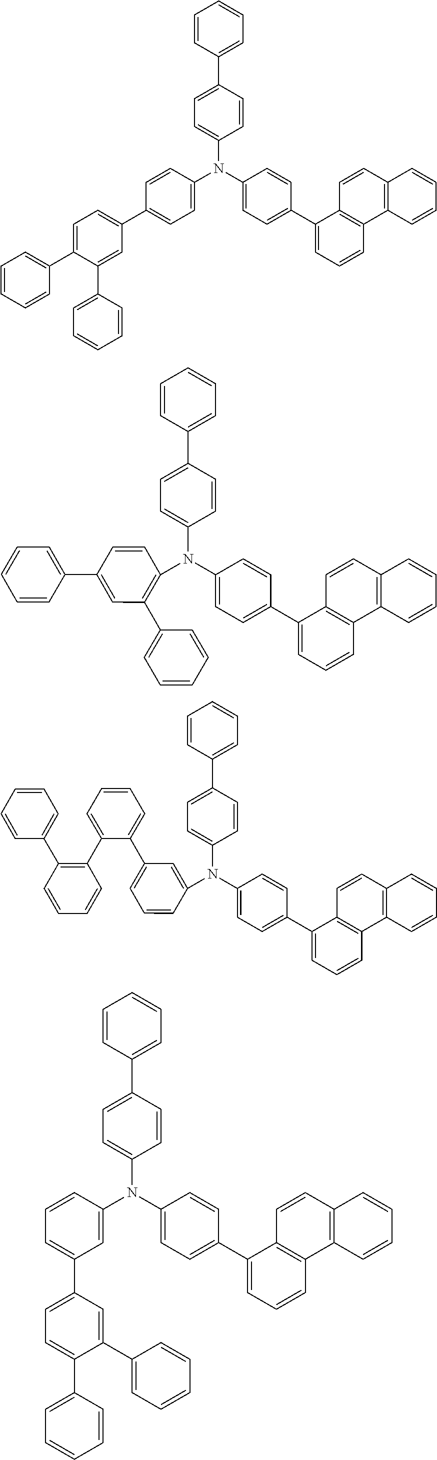

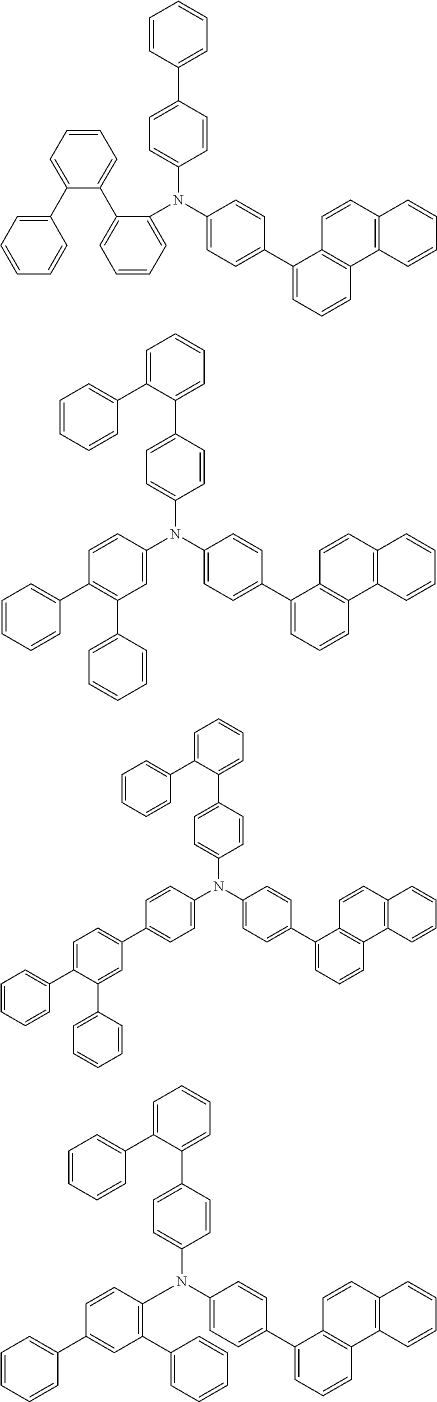 [SMILES: c1ccc(-c2ccc(N(c3ccc(-c4cccc5c4ccc4ccccc45)cc3)c3ccccc3-c3ccccc3-c3ccccc3)cc2)cc1 ; c1ccc(-c2ccccc2-c2ccc(N(c3ccc(-c4cccc5c4ccc4ccccc45)cc3)c3ccc(-c4ccccc4)c(-c4ccccc4)c3)cc2)cc1 ; c1ccc(-c2ccccc2-c2ccc(N(c3ccc(-c4ccc(-c5ccccc5)c(-c5ccccc5)c4)cc3)c3ccc(-c4cccc5c4ccc4ccccc45)cc3)cc2)cc1 ; c1ccc(-c2ccc(N(c3ccc(-c4ccccc4-c4ccccc4)cc3)c3ccc(-c4cccc5c4ccc4ccccc45)cc3)c(-c3ccccc3)c2)cc1]

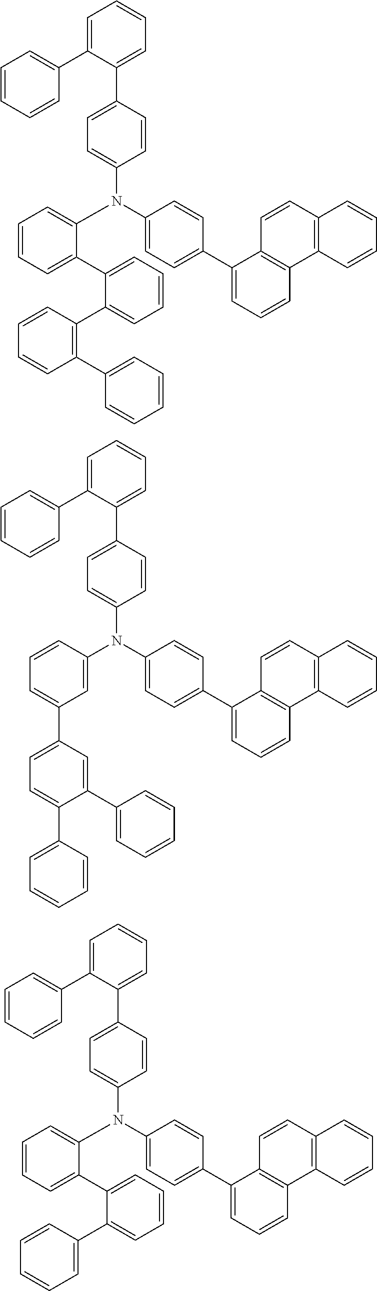

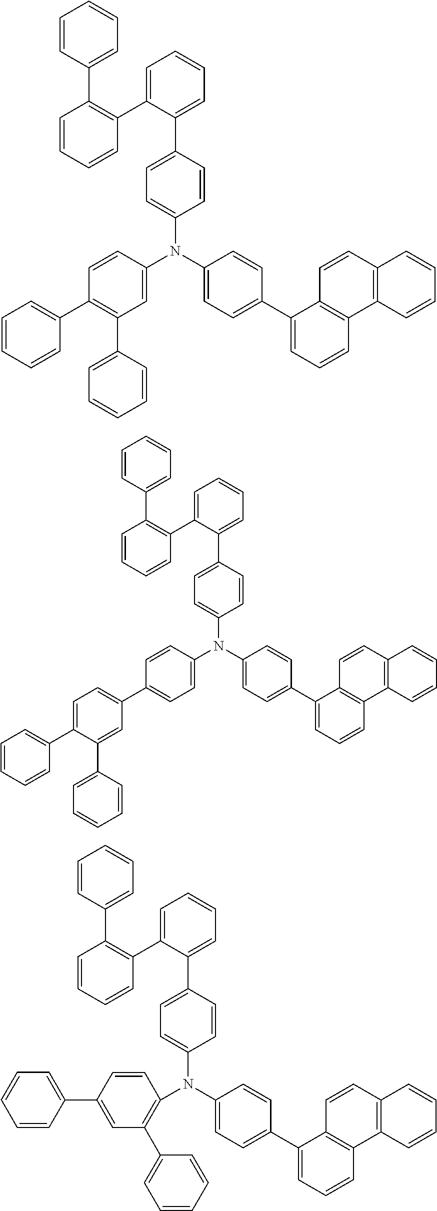

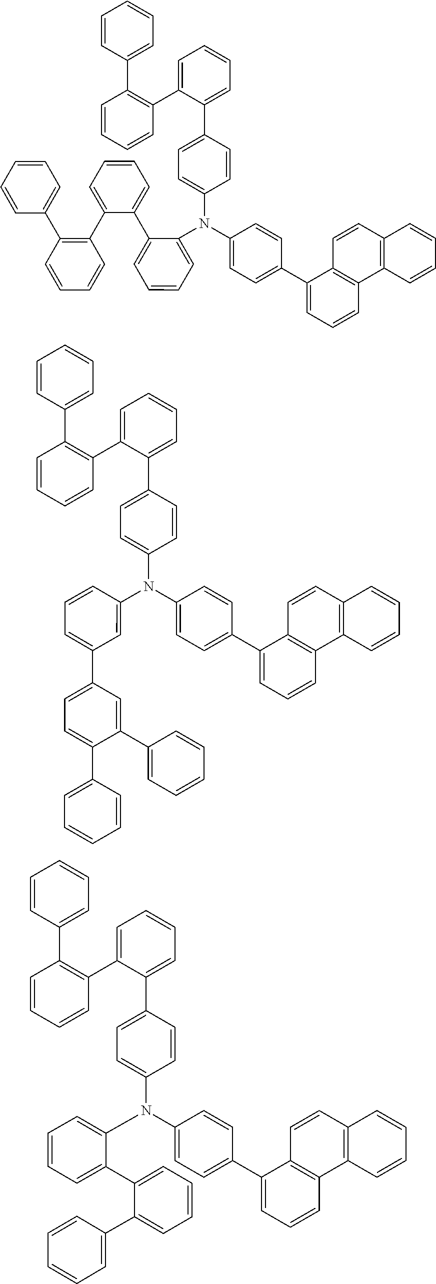

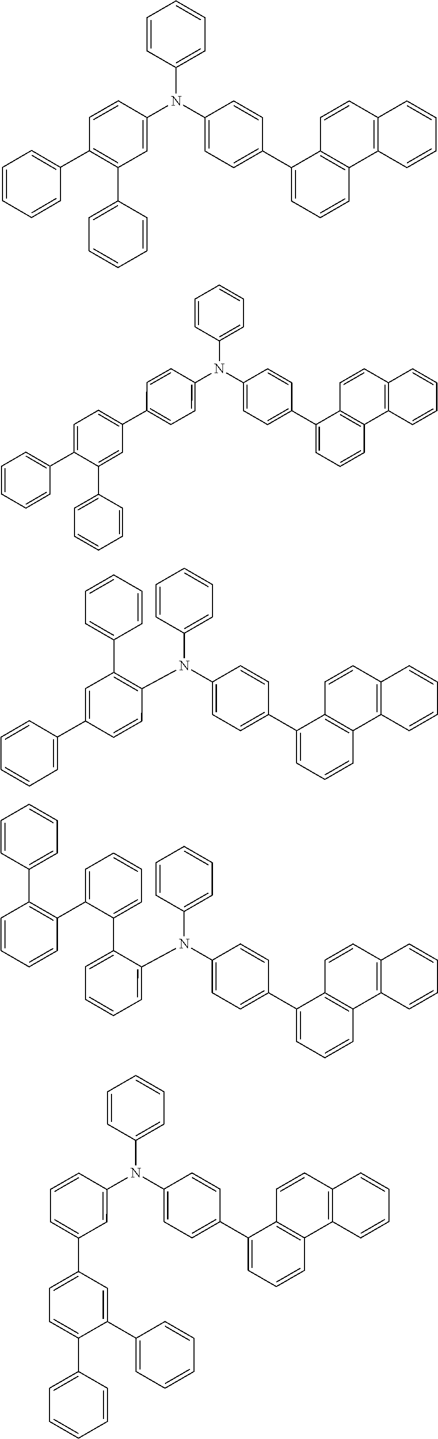

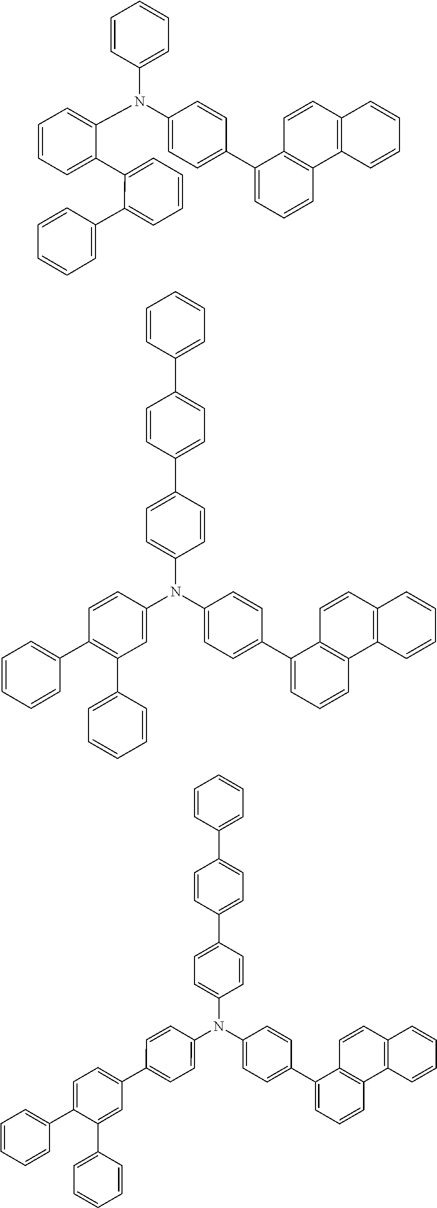 [SMILES: c1ccc(-c2ccccc2-c2ccccc2N(c2ccccc2)c2ccc(-c3cccc4c3ccc3ccccc34)cc2)cc1 ; c1ccc(-c2ccc(-c3ccc(N(c4ccc(-c5cccc6c5ccc5ccccc56)cc4)c4ccc(-c5ccccc5)c(-c5ccccc5)c4)cc3)cc2)cc1 ; c1ccc(-c2ccc(-c3ccc(N(c4ccc(-c5ccc(-c6ccccc6)c(-c6ccccc6)c5)cc4)c4ccc(-c5cccc6c5ccc5ccccc56)cc4)cc3)cc2)cc1]

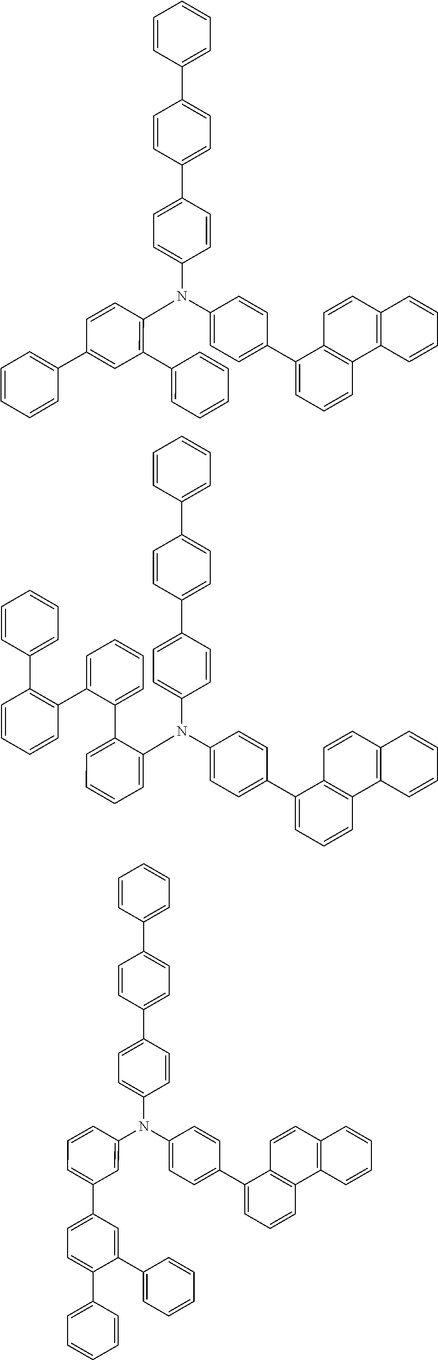

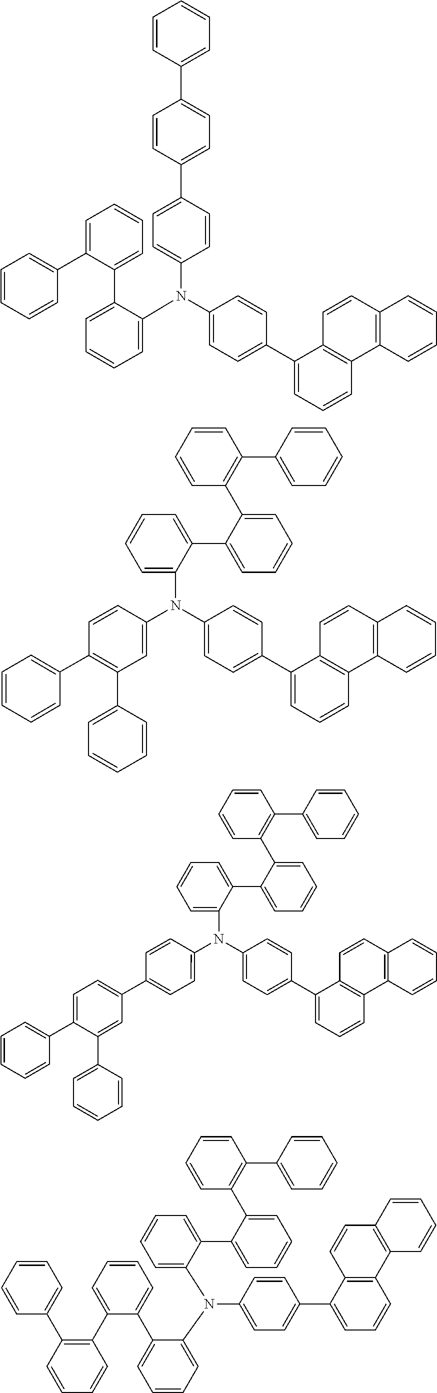

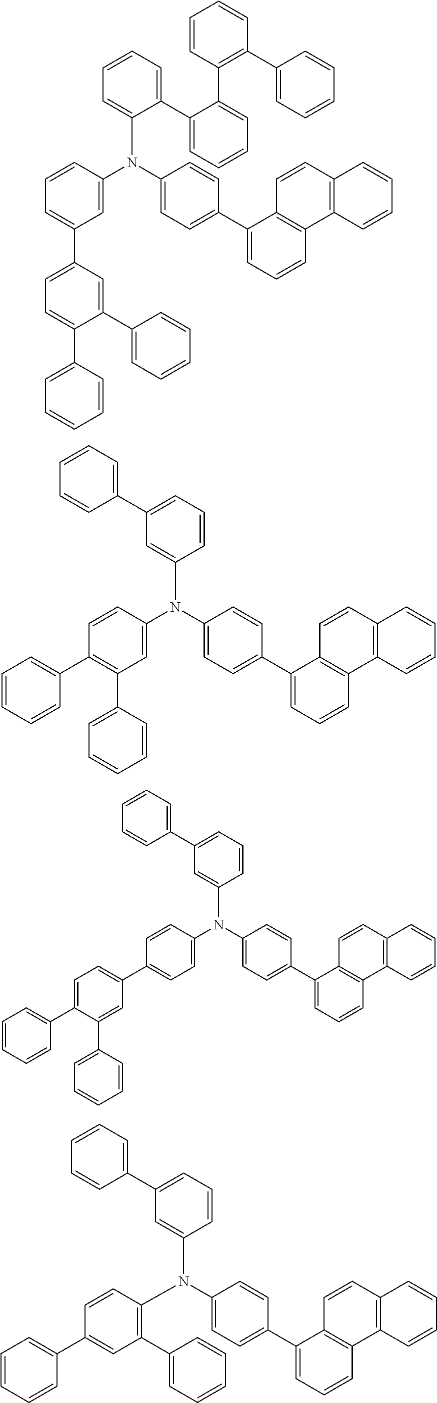

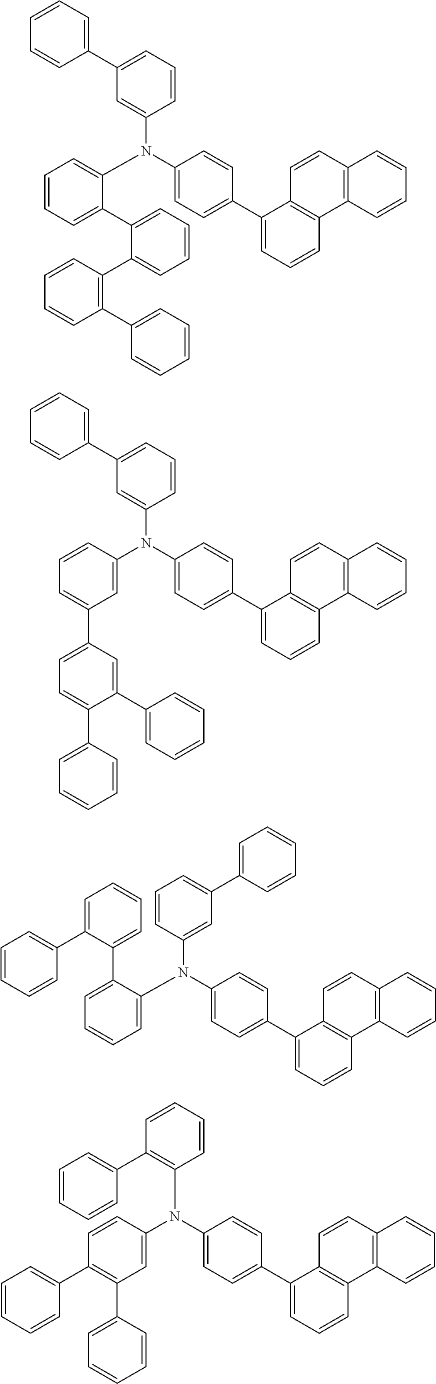 [SMILES: c1ccc(-c2cccc(N(c3ccc(-c4cccc5c4ccc4ccccc45)cc3)c3ccccc3-c3ccccc3-c3ccccc3-c3ccccc3)c2)cc1 ; c1ccc(-c2cccc(N(c3ccc(-c4cccc5c4ccc4ccccc45)cc3)c3cccc(-c4ccc(-c5ccccc5)c(-c5ccccc5)c4)c3)c2)cc1 ; c1ccc(-c2cccc(N(c3ccc(-c4cccc5c4ccc4ccccc45)cc3)c3ccccc3-c3ccccc3-c3ccccc3)c2)cc1 ; c1ccc(-c2ccc(N(c3ccc(-c4cccc5c4ccc4ccccc45)cc3)c3ccccc3-c3ccccc3)cc2-c2ccccc2)cc1]

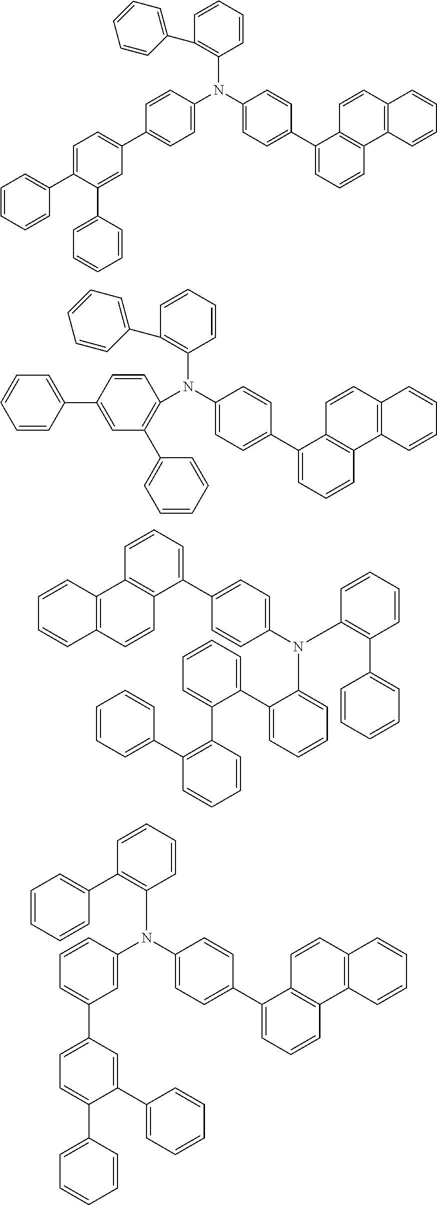

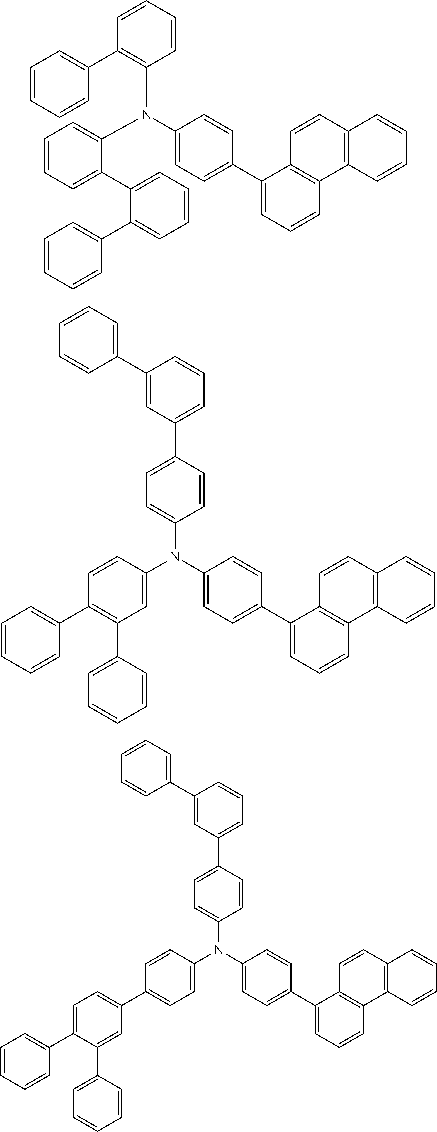

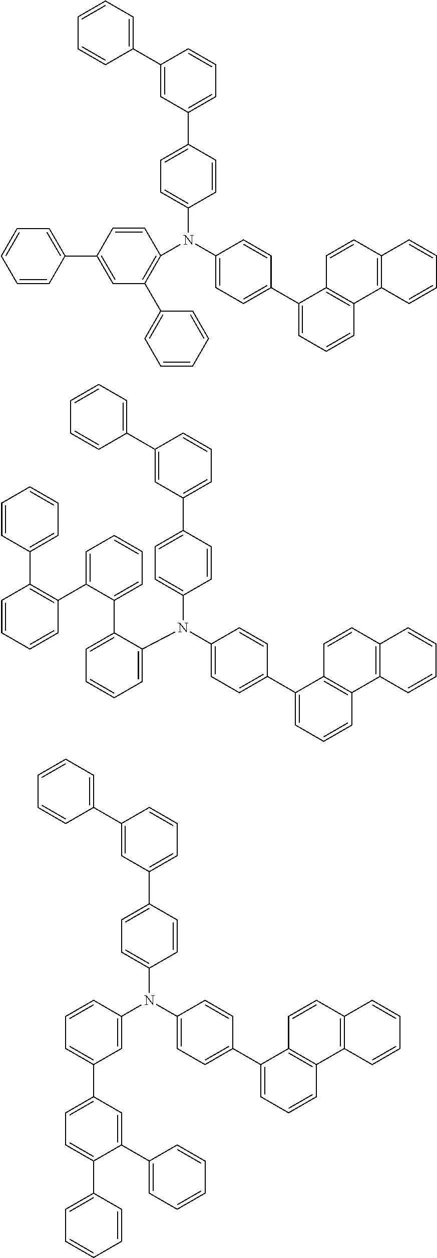 [SMILES: c1ccc(-c2cccc(-c3ccc(N(c4ccc(-c5cccc6c5ccc5ccccc56)cc4)c4ccc(-c5ccccc5)cc4-c4ccccc4)cc3)c2)cc1 ; c1ccc(-c2cccc(-c3ccc(N(c4ccc(-c5cccc6c5ccc5ccccc56)cc4)c4ccccc4-c4ccccc4-c4ccccc4-c4ccccc4)cc3)c2)cc1 ; c1ccc(-c2cccc(-c3ccc(N(c4ccc(-c5cccc6c5ccc5ccccc56)cc4)c4cccc(-c5ccc(-c6ccccc6)c(-c6ccccc6)c5)c4)cc3)c2)cc1]

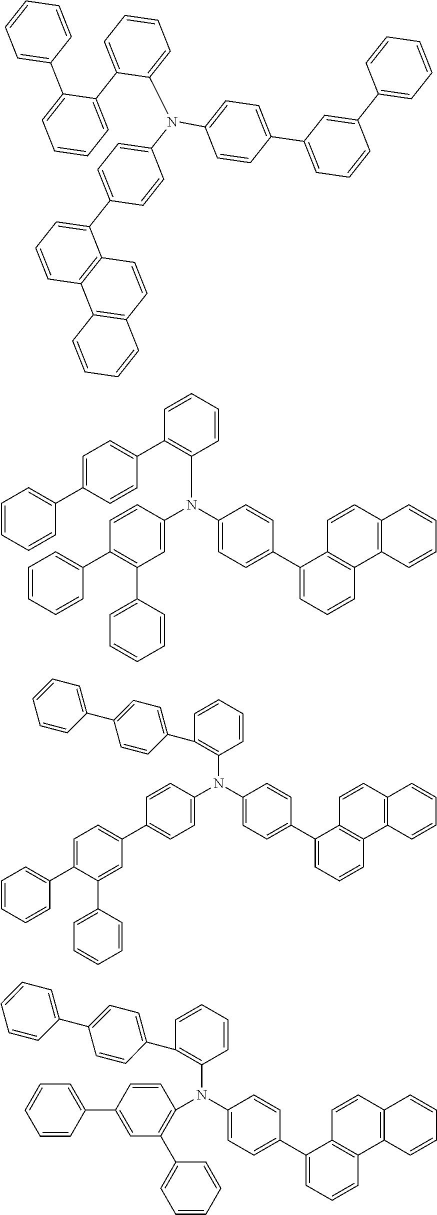

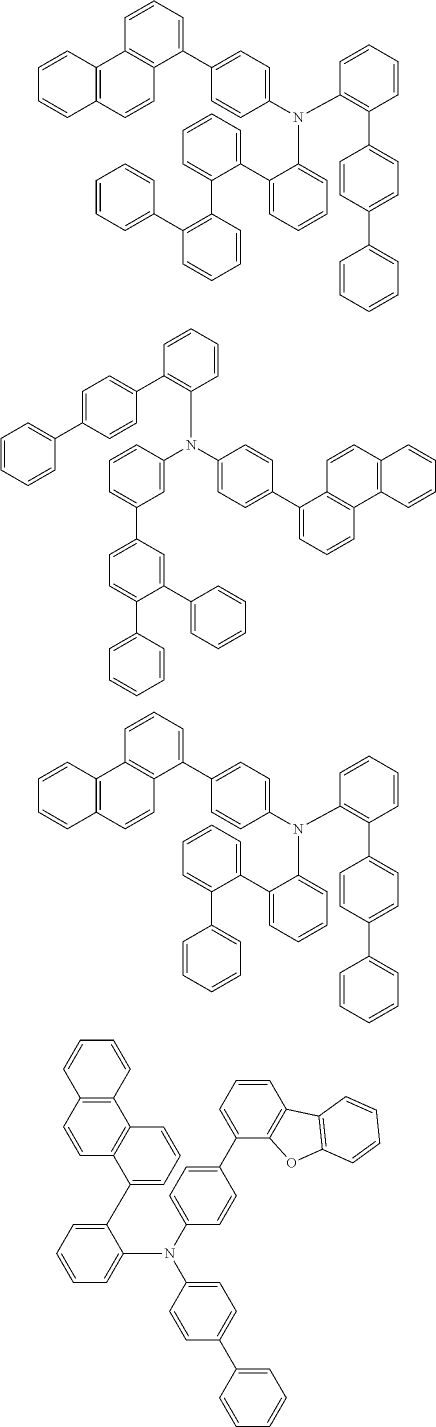

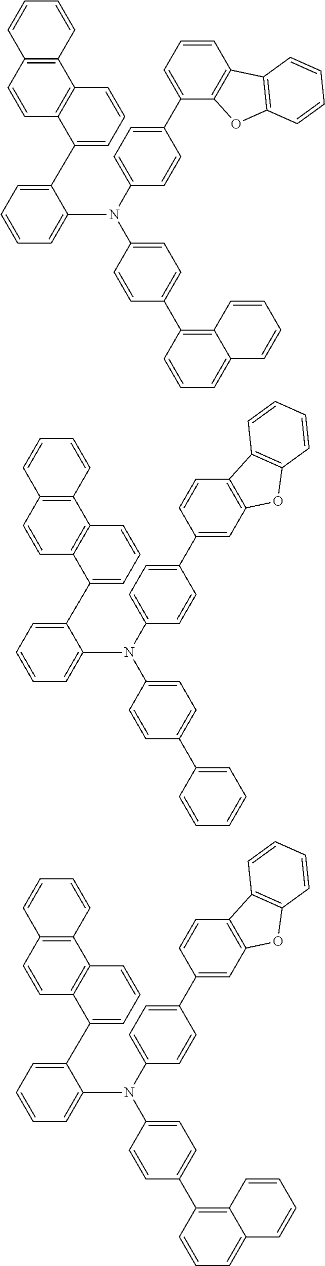

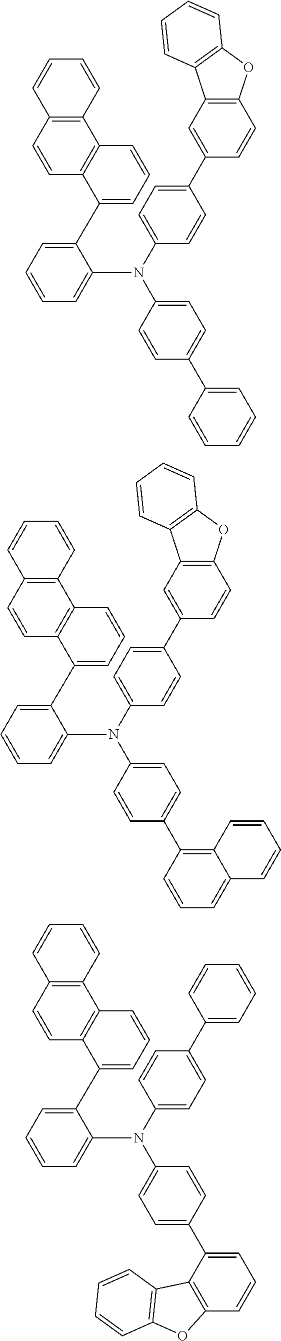

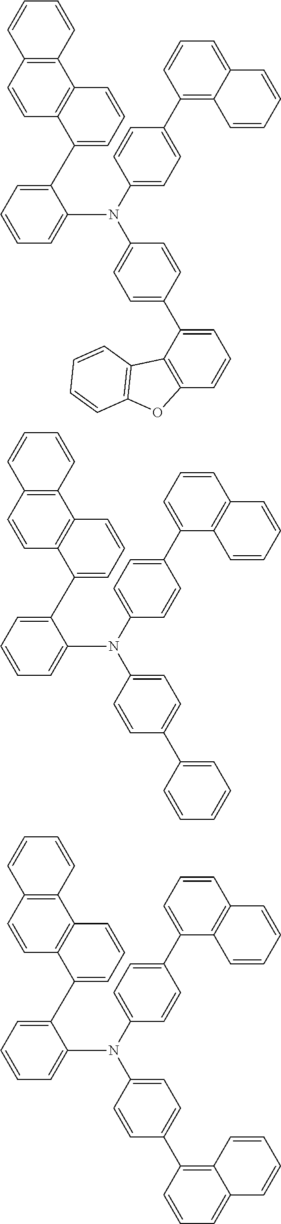

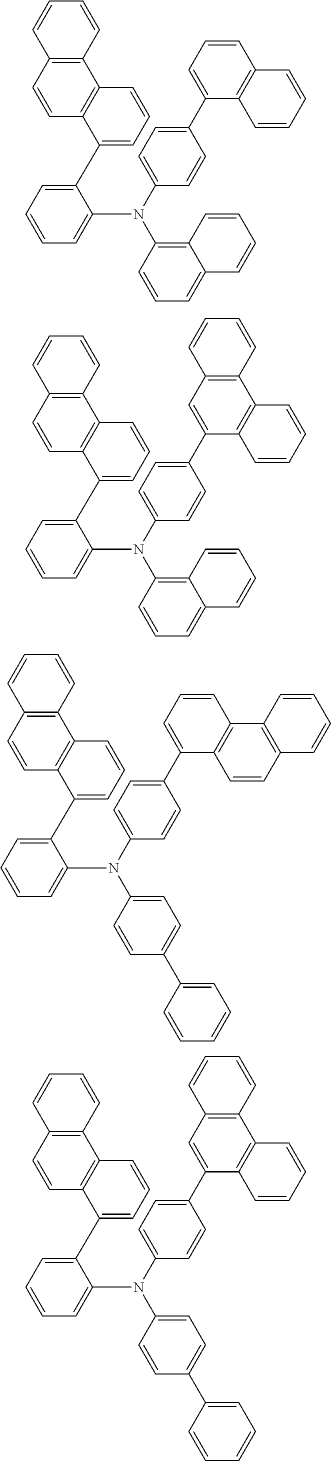

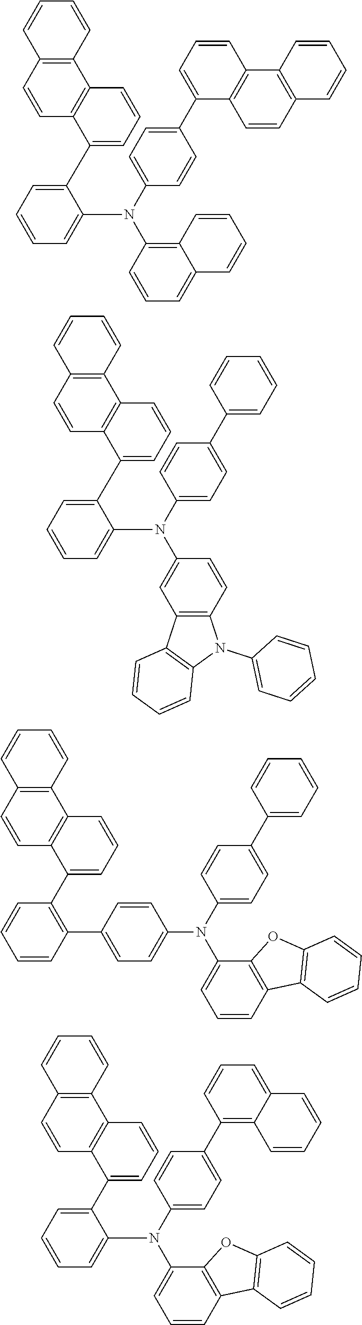

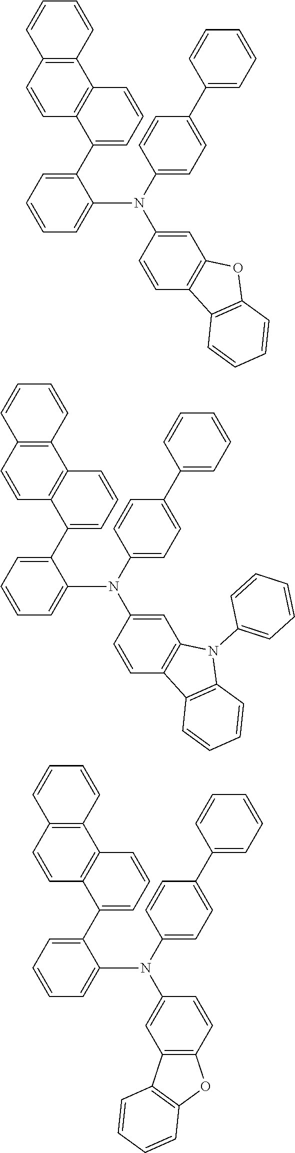

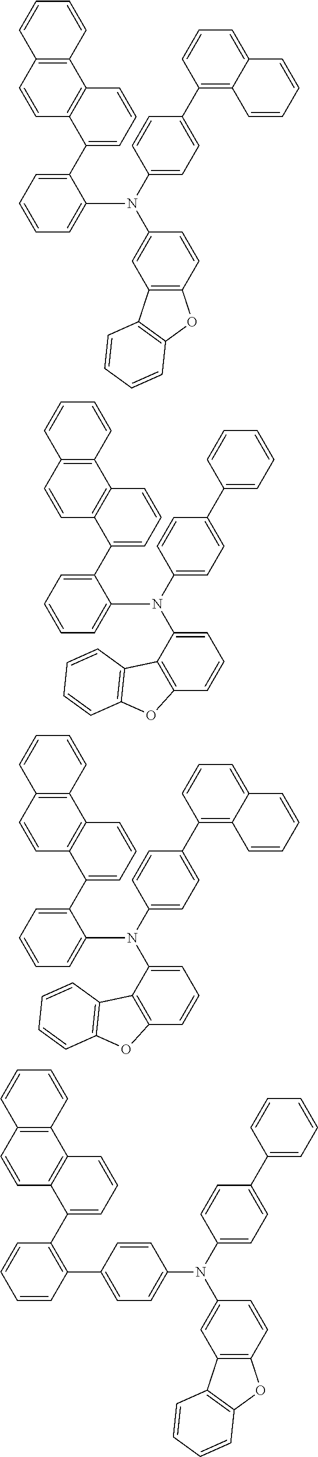

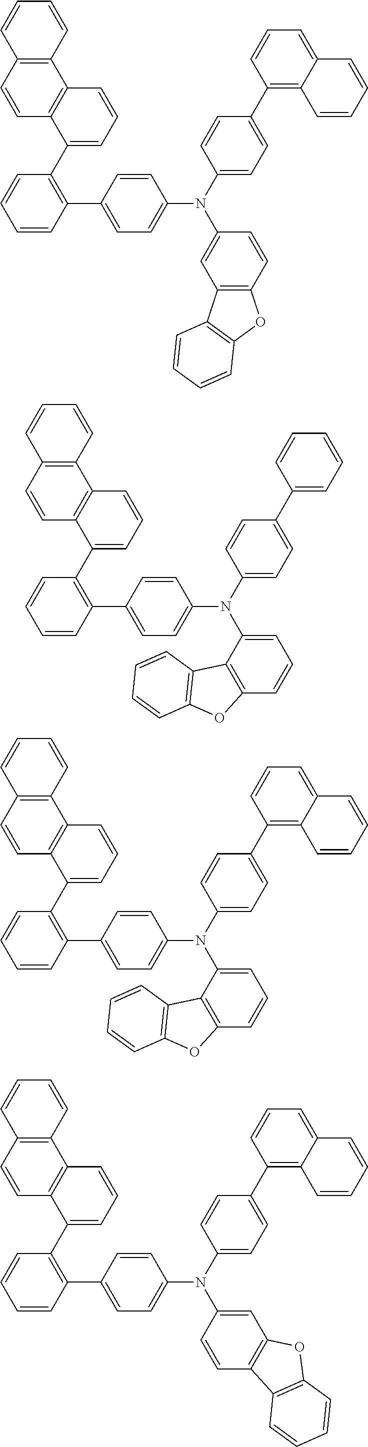 [SMILES: c1ccc(-c2cccc3c2ccc2ccccc23)c(-c2ccc(N(c3ccc(-c4cccc5ccccc45)cc3)c3ccc4oc5ccccc5c4c3)cc2)c1 ; c1ccc(-c2ccc(N(c3ccc(-c4ccccc4-c4cccc5c4ccc4ccccc45)cc3)c3cccc4oc5ccccc5c34)cc2)cc1 ; c1ccc(-c2cccc3c2ccc2ccccc23)c(-c2ccc(N(c3ccc(-c4cccc5ccccc45)cc3)c3cccc4oc5ccccc5c34)cc2)c1 ; c1ccc(-c2cccc3c2ccc2ccccc23)c(-c2ccc(N(c3ccc(-c4cccc5ccccc45)cc3)c3ccc4c(c3)oc3ccccc34)cc2)c1]

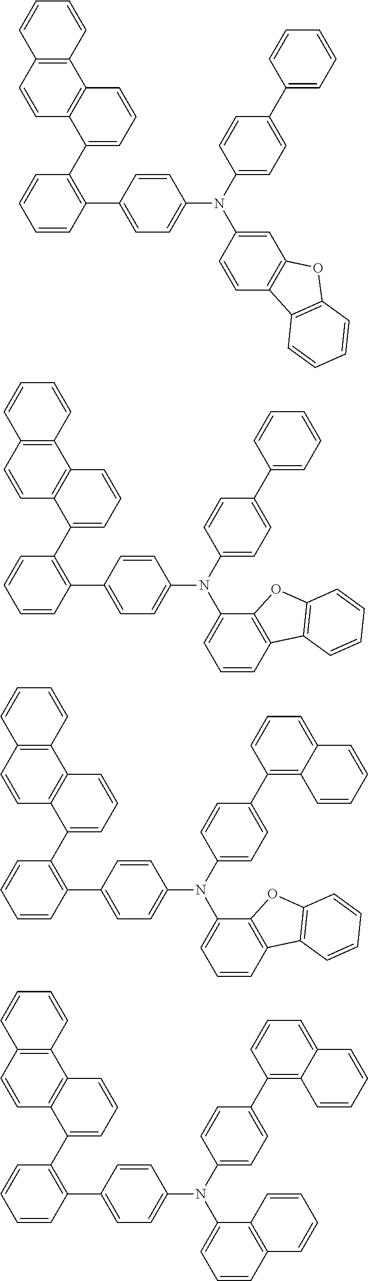

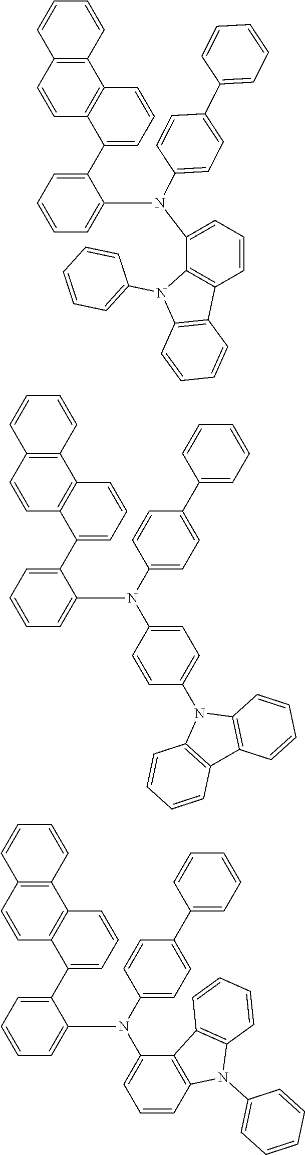 [SMILES: c1ccc(-c2ccc(N(c3ccccc3-c3cccc4c3ccc3ccccc34)c3cccc4c5ccccc5n(-c5ccccc5)c34)cc2)cc1 ; c1ccc(-c2ccc(N(c3ccc(-n4c5ccccc5c5ccccc54)cc3)c3ccccc3-c3cccc4c3ccc3ccccc34)cc2)cc1 ; c1ccc(-c2ccc(N(c3ccccc3-c3cccc4c3ccc3ccccc34)c3cccc4c3c3ccccc3n4-c3ccccc3)cc2)cc1]

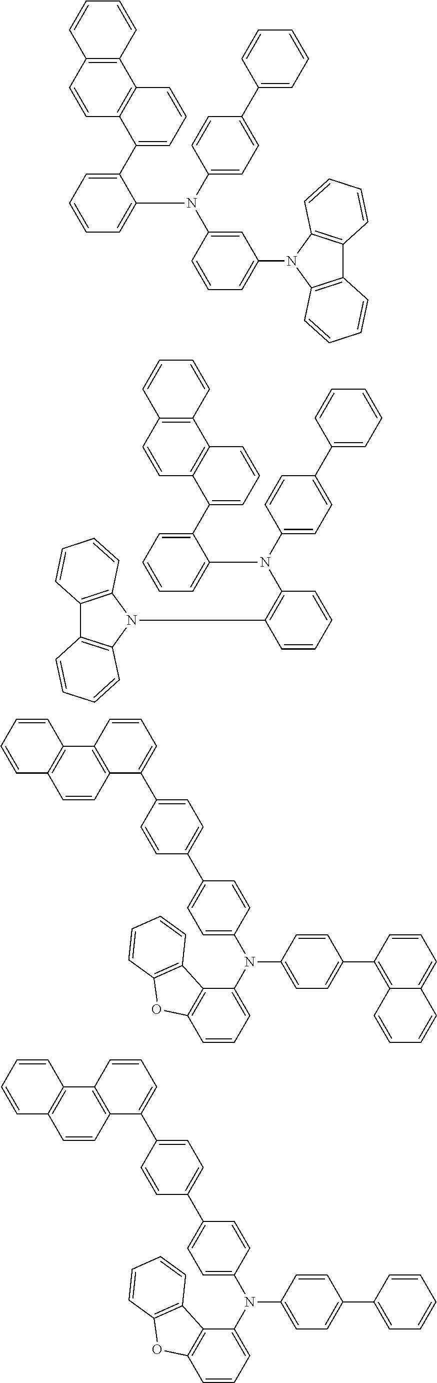

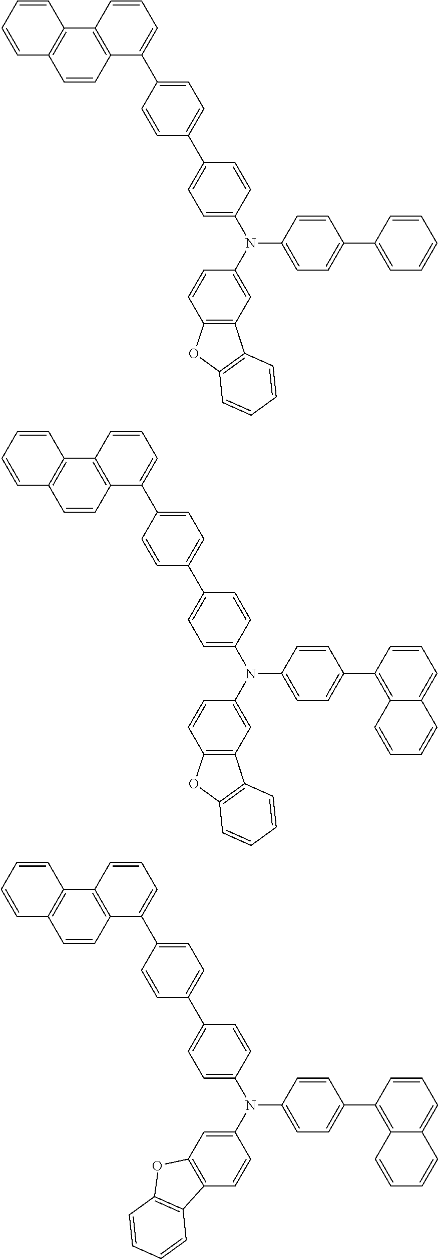 [SMILES: c1ccc(-c2ccc(N(c3ccc(-c4ccc(-c5cccc6c5ccc5ccccc56)cc4)cc3)c3ccc4oc5ccccc5c4c3)cc2)cc1 ; c1ccc2c(-c3ccc(N(c4ccc(-c5ccc(-c6cccc7c6ccc6ccccc67)cc5)cc4)c4ccc5oc6ccccc6c5c4)cc3)cccc2c1 ; c1ccc2c(-c3ccc(N(c4ccc(-c5ccc(-c6cccc7c6ccc6ccccc67)cc5)cc4)c4ccc5c(c4)oc4ccccc45)cc3)cccc2c1]

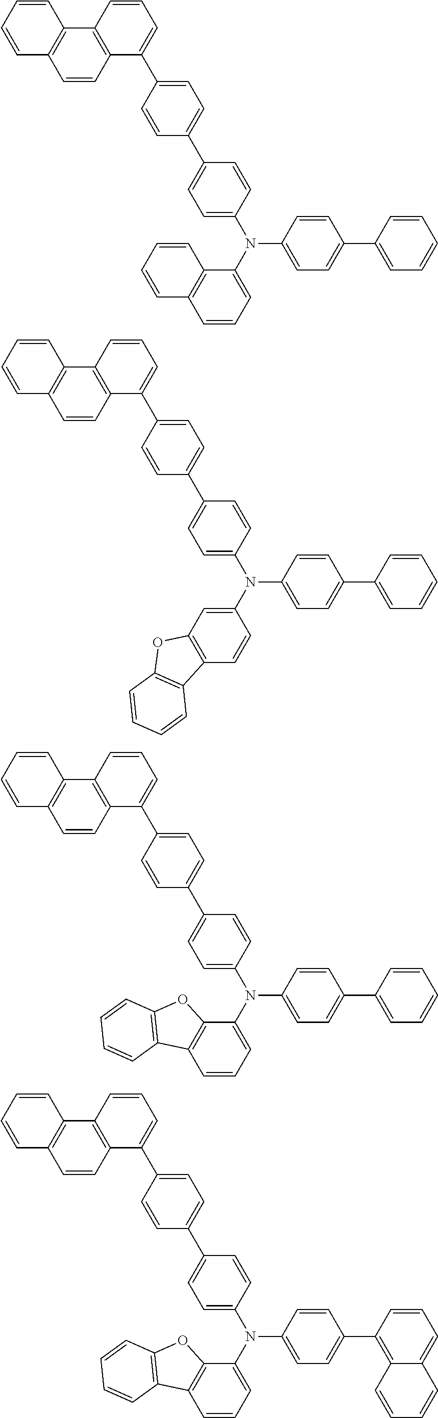

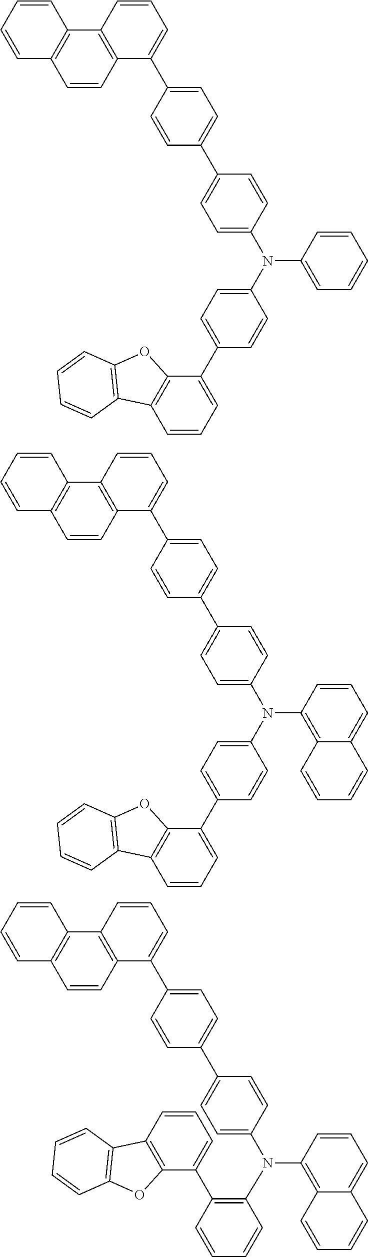

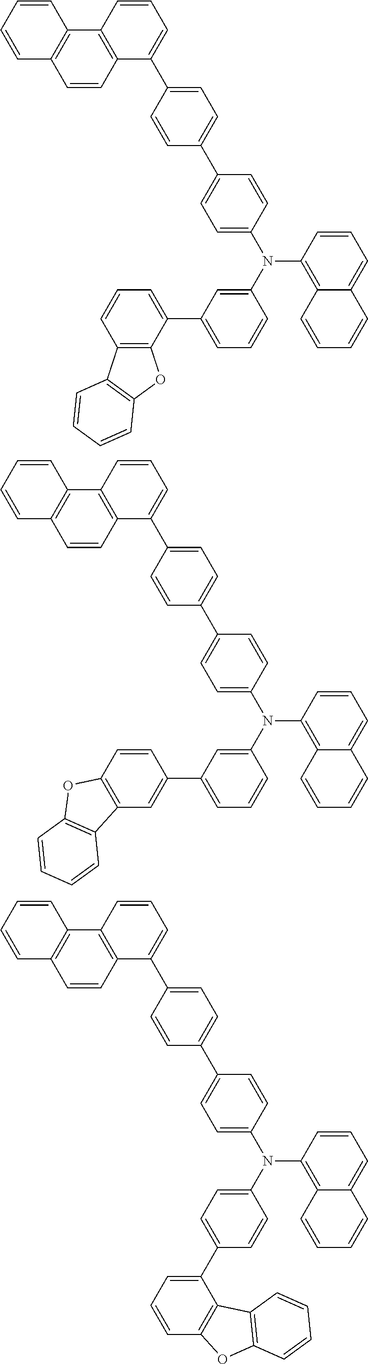

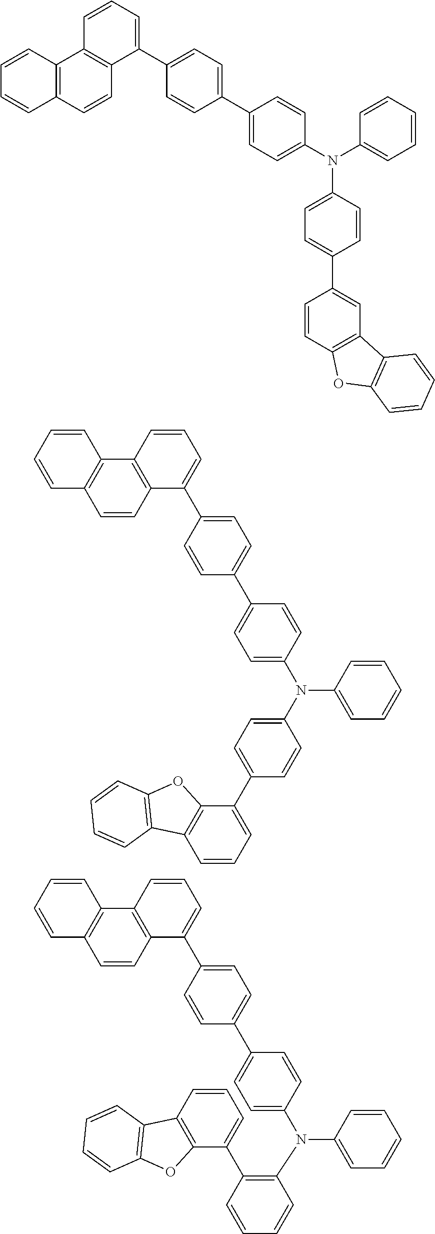

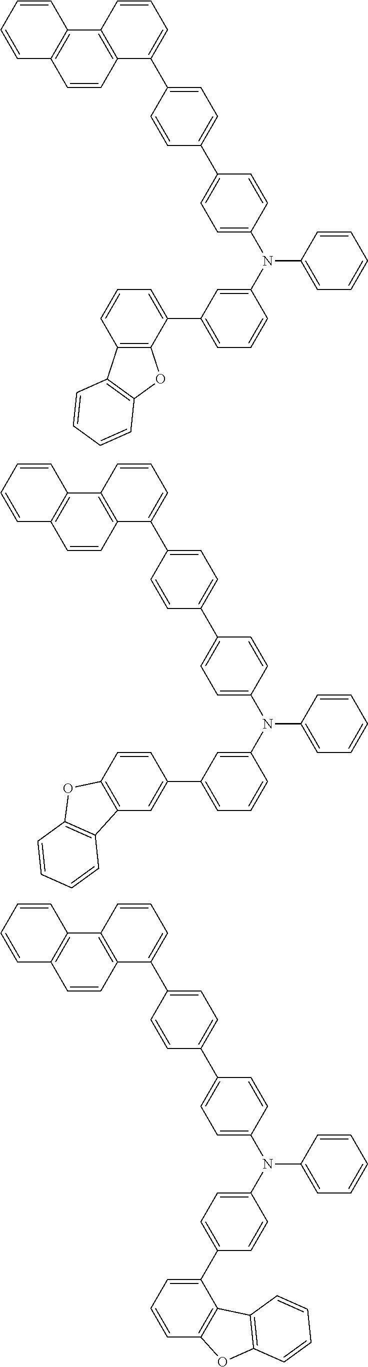

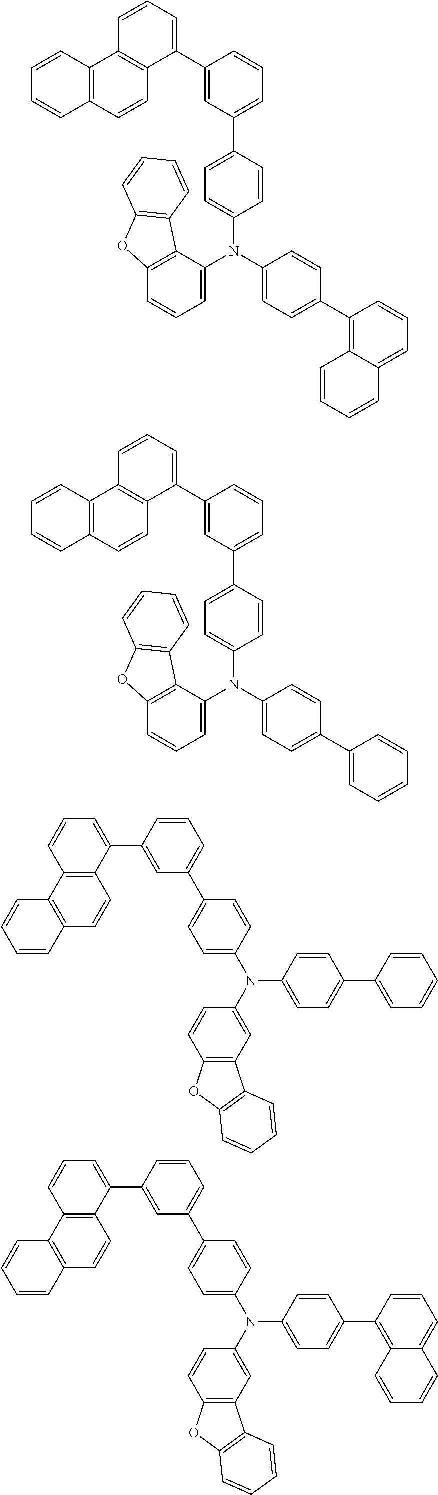

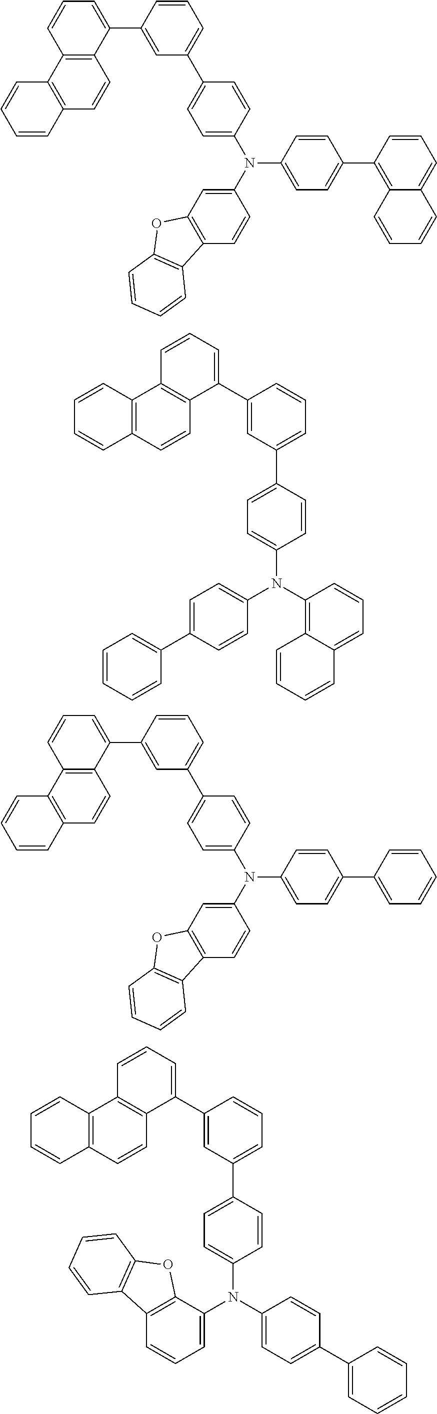

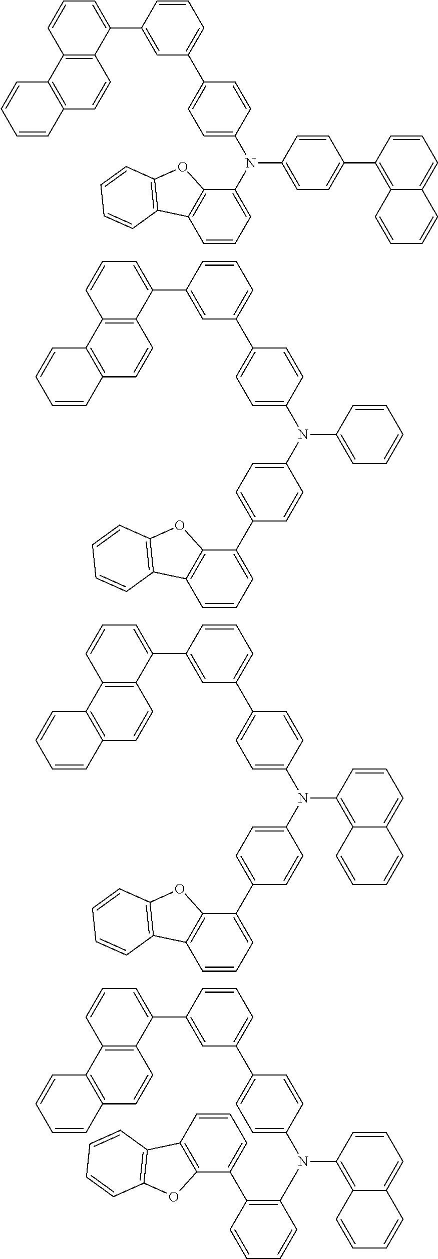

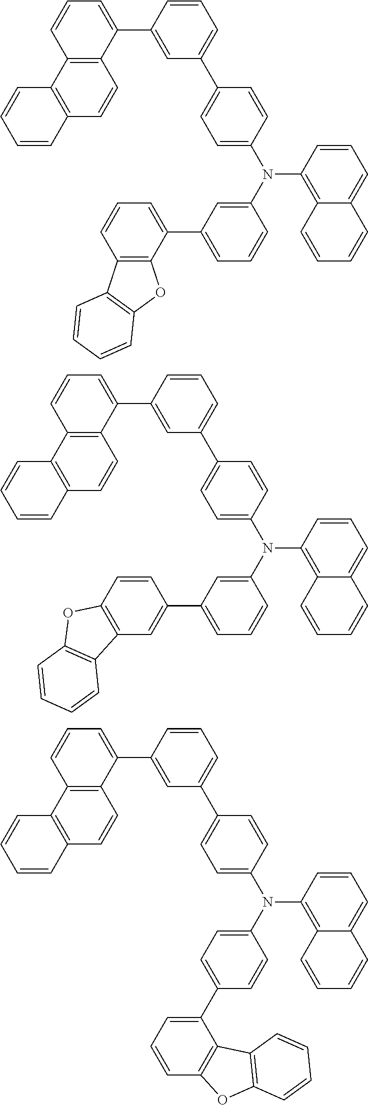

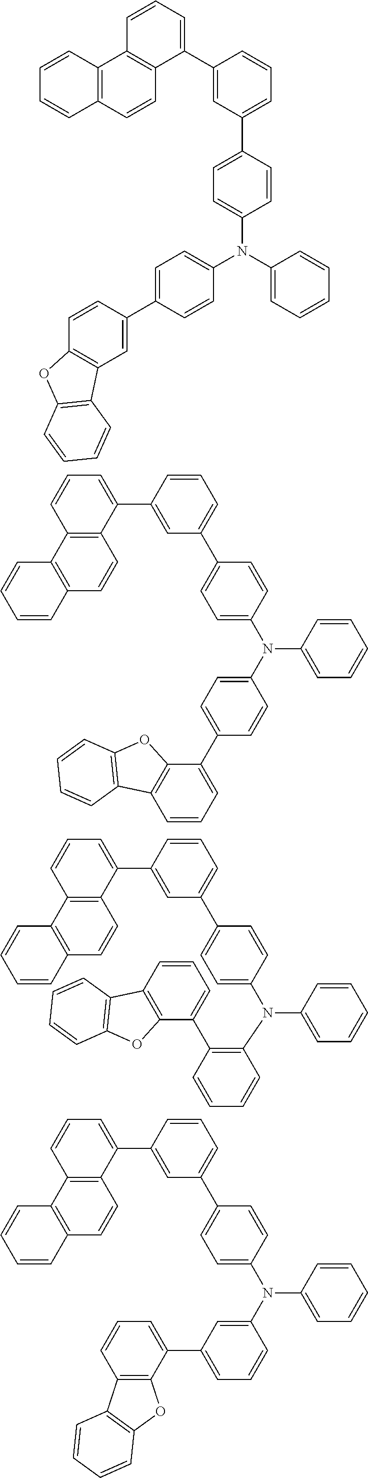

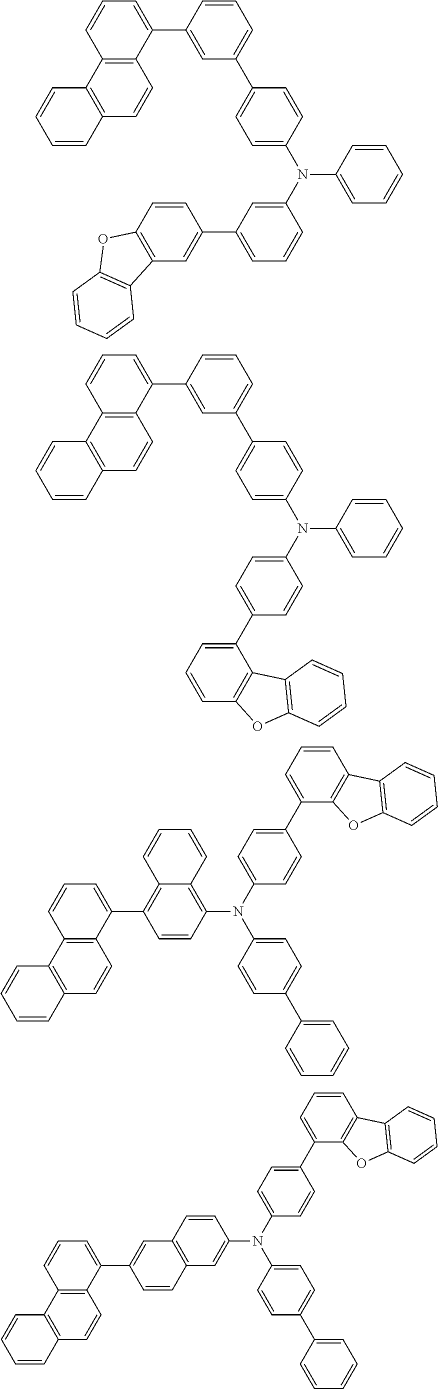

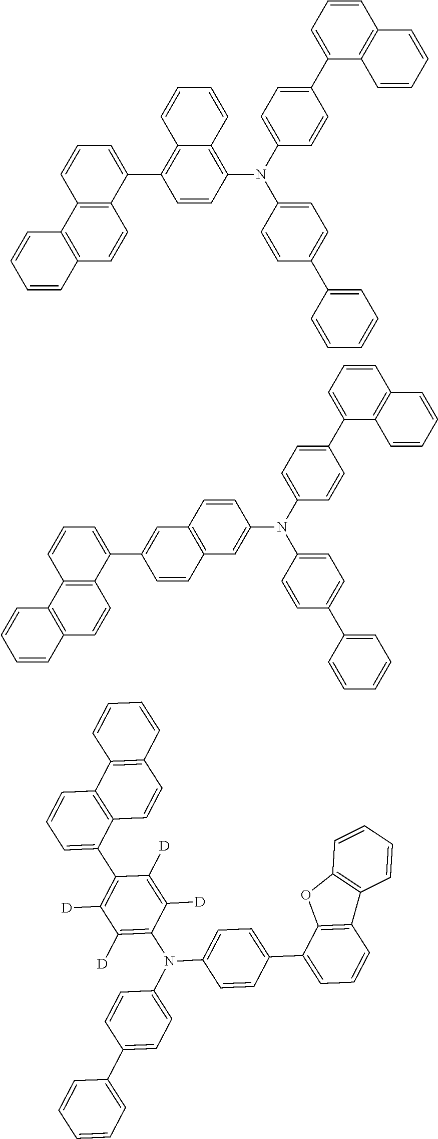

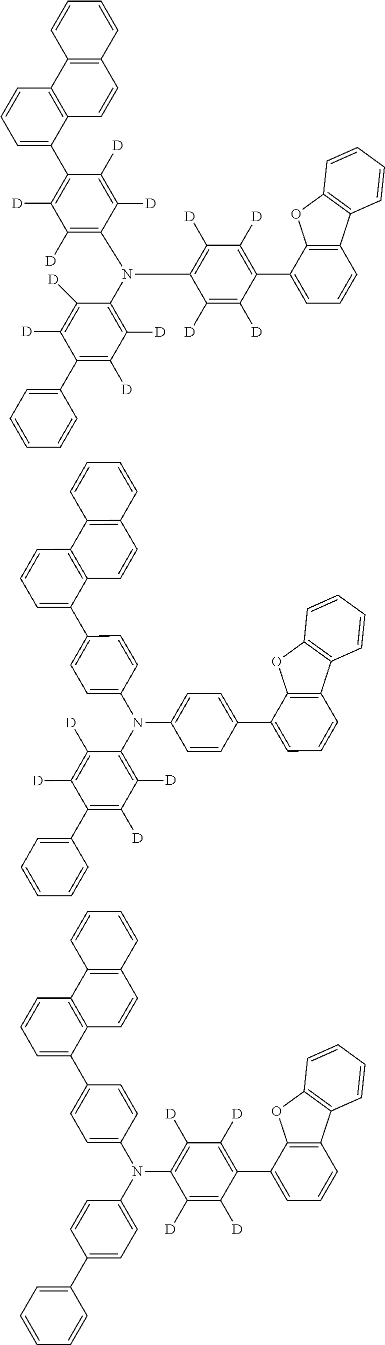

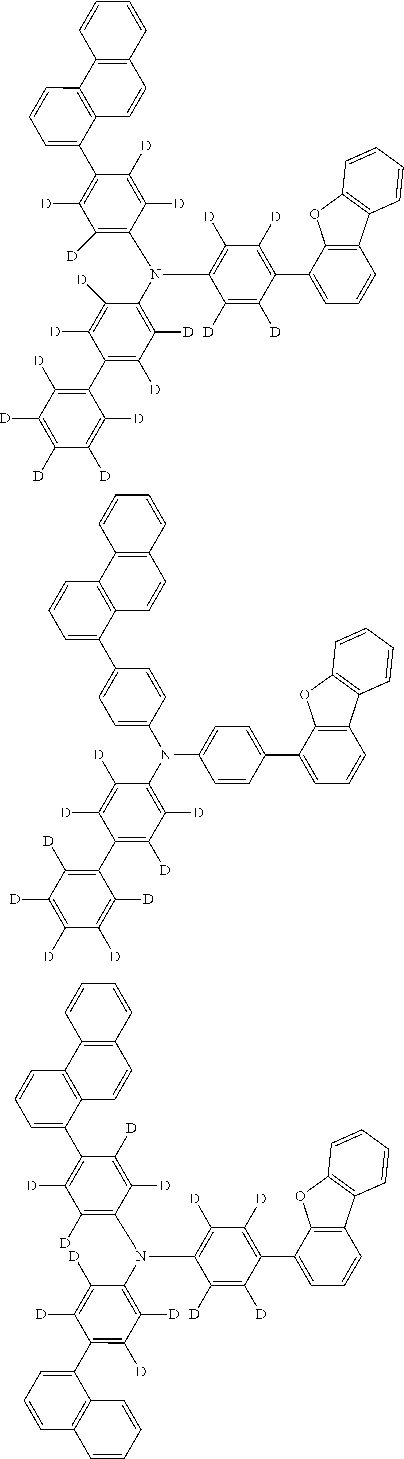

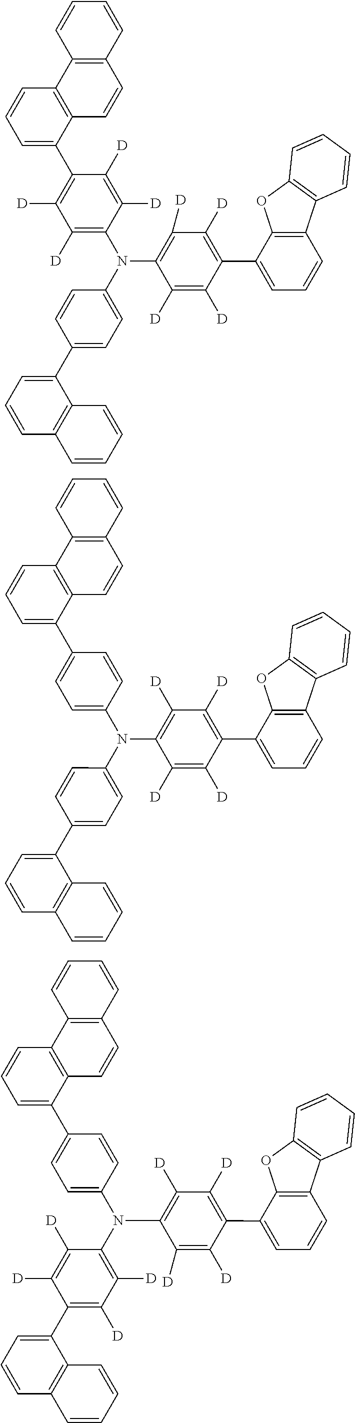

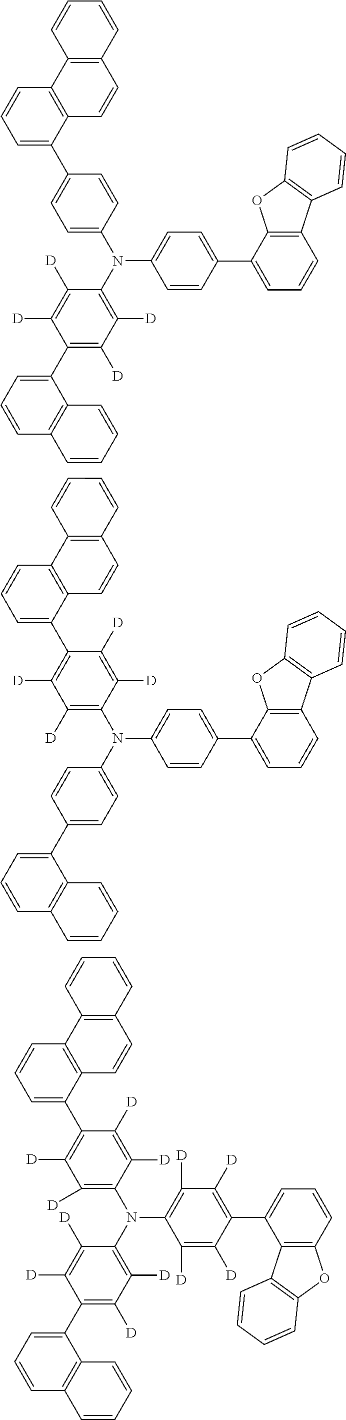

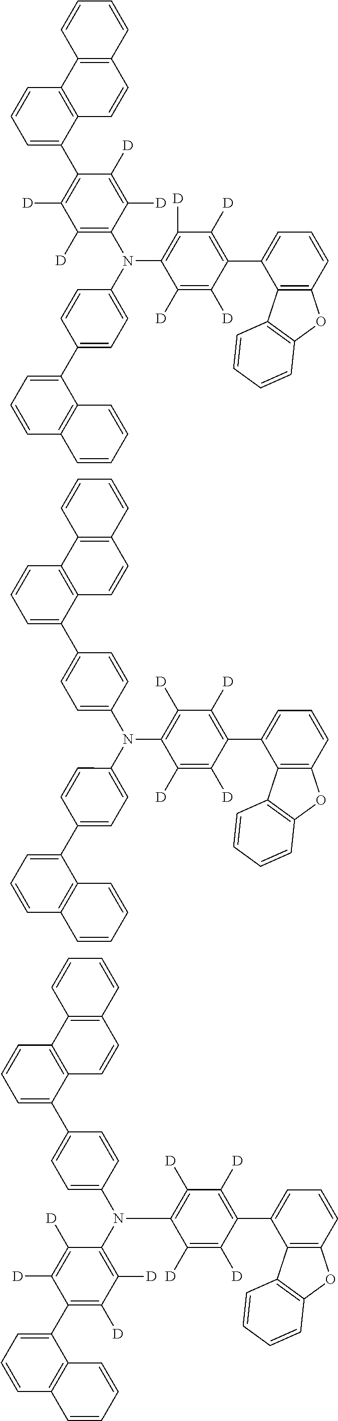

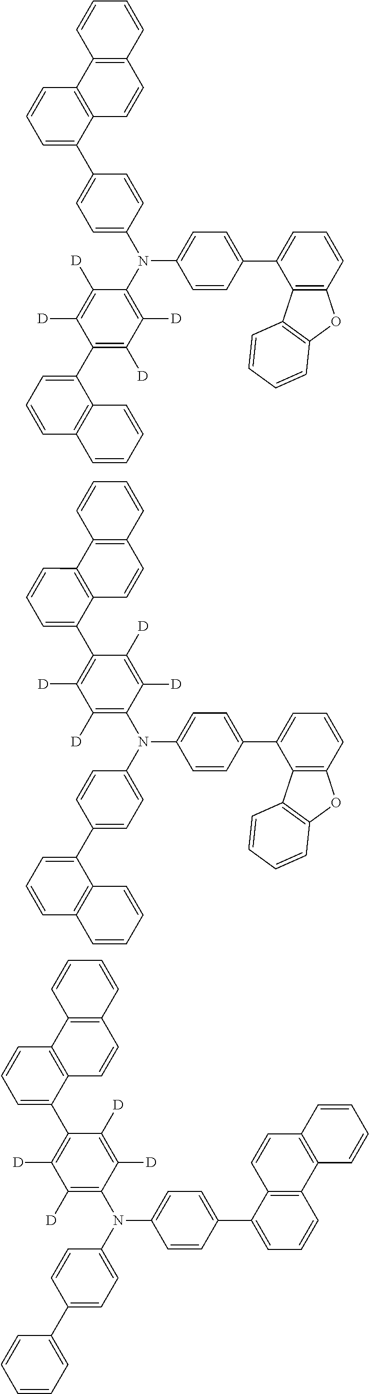

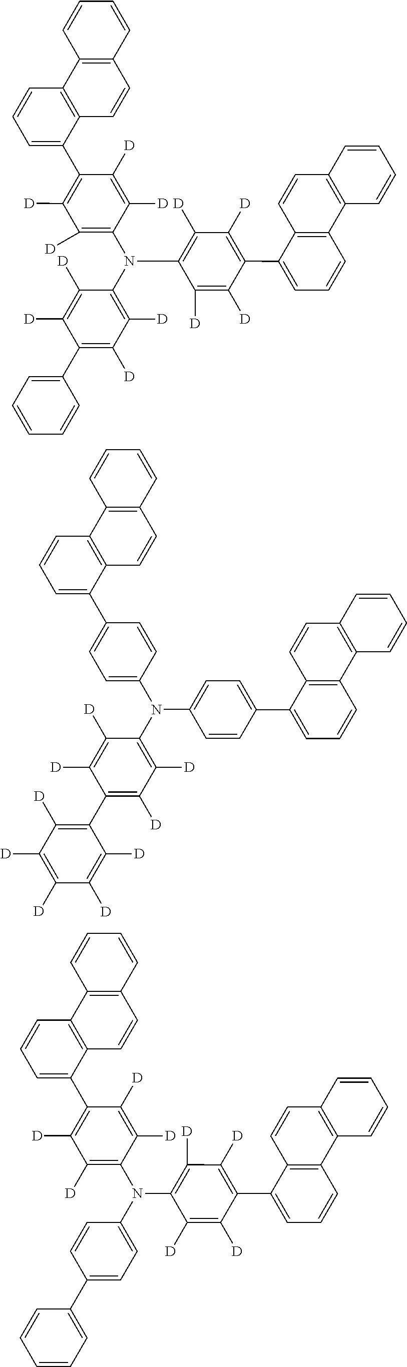

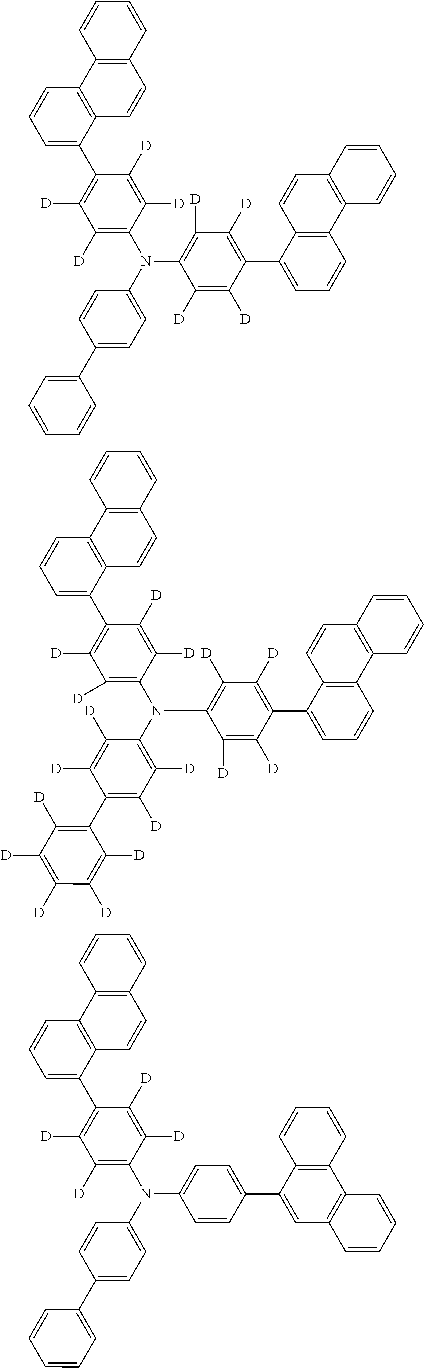

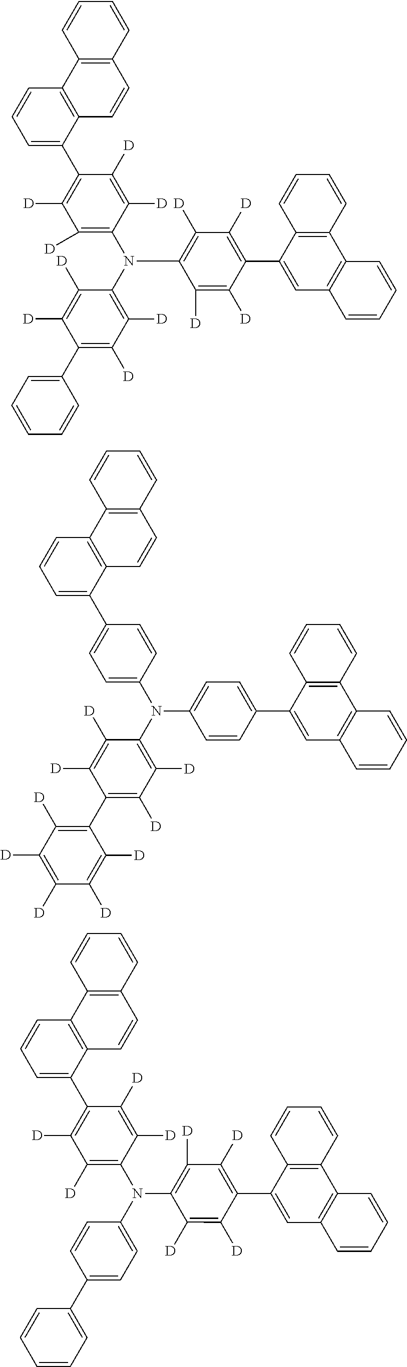

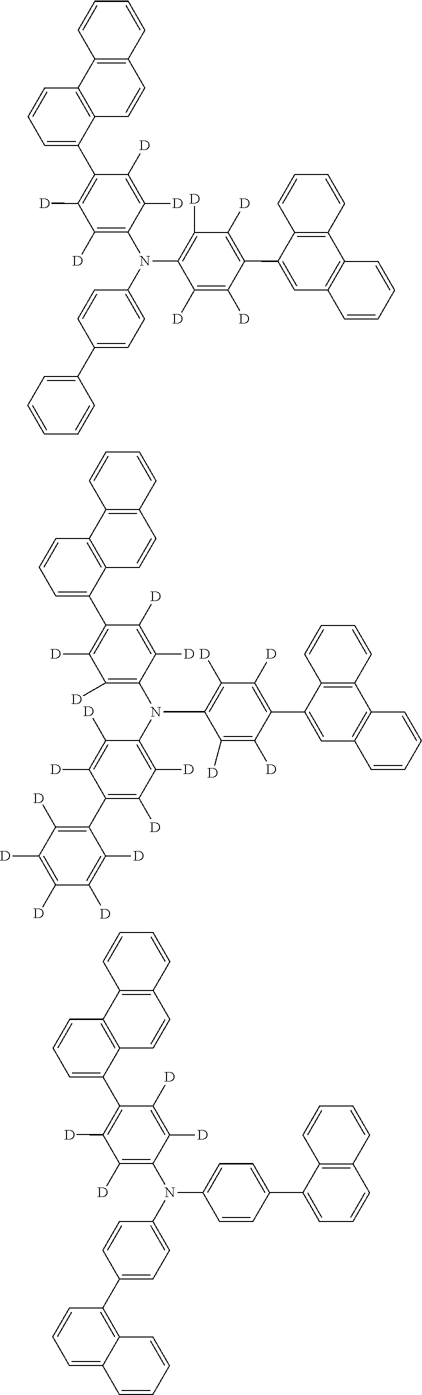

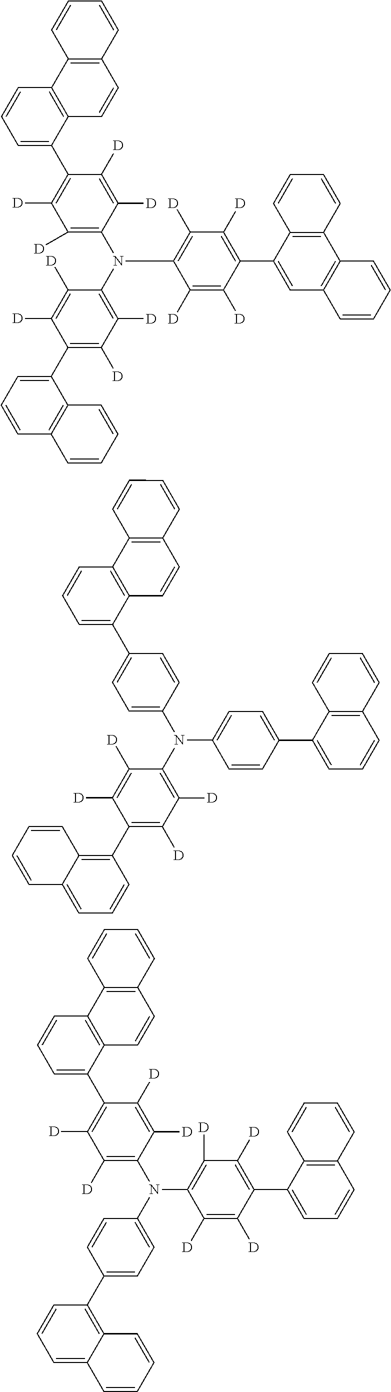

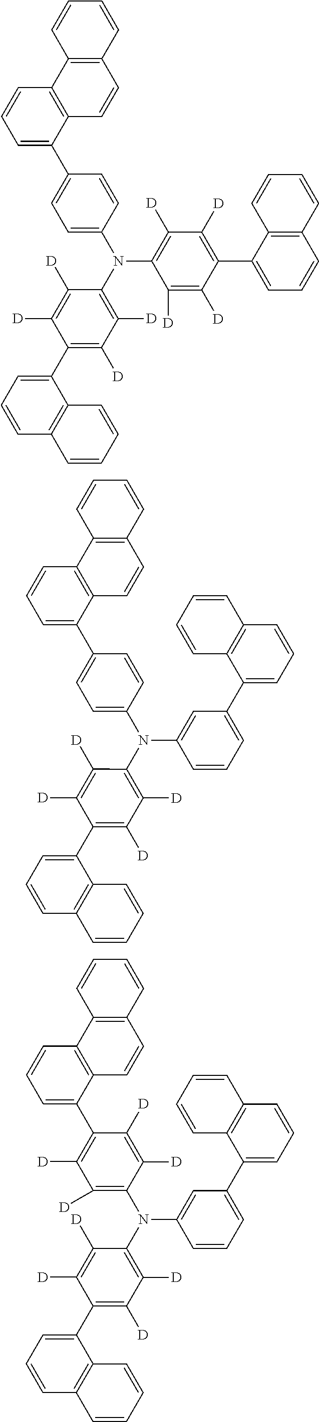

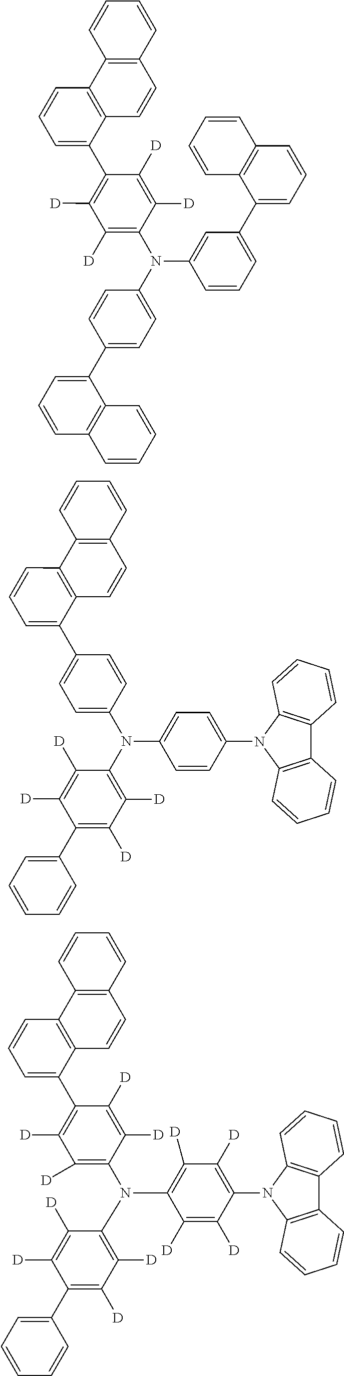

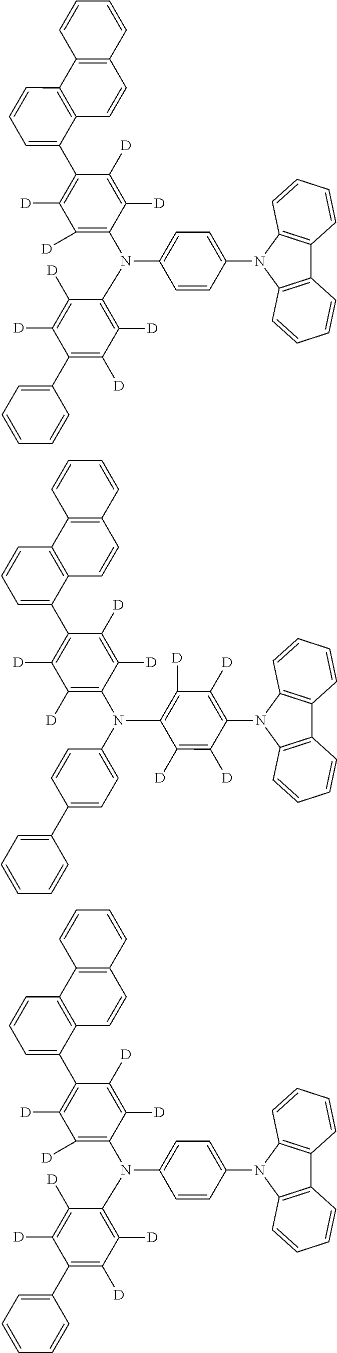

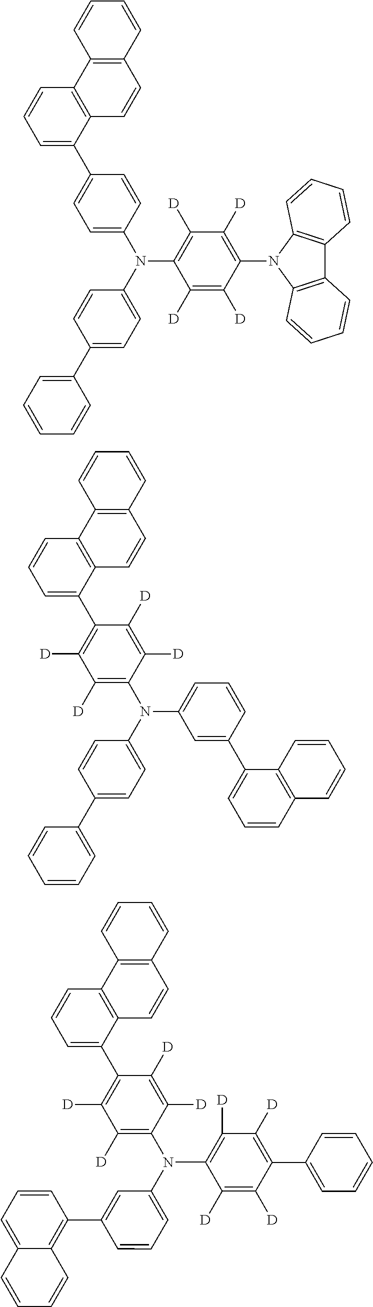

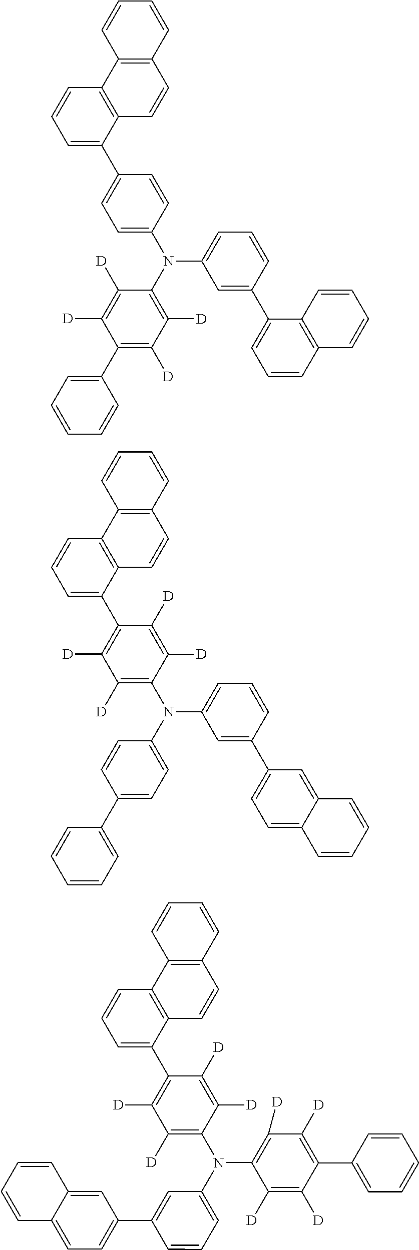

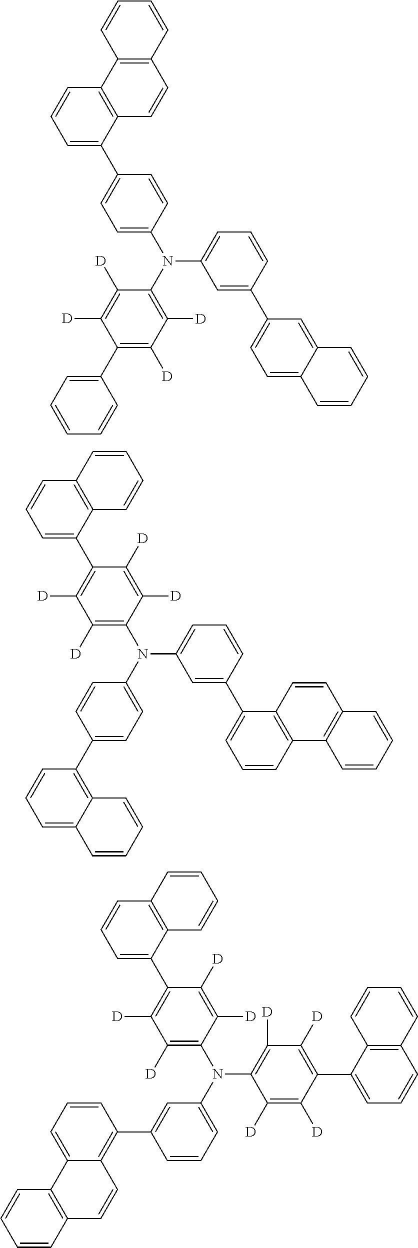

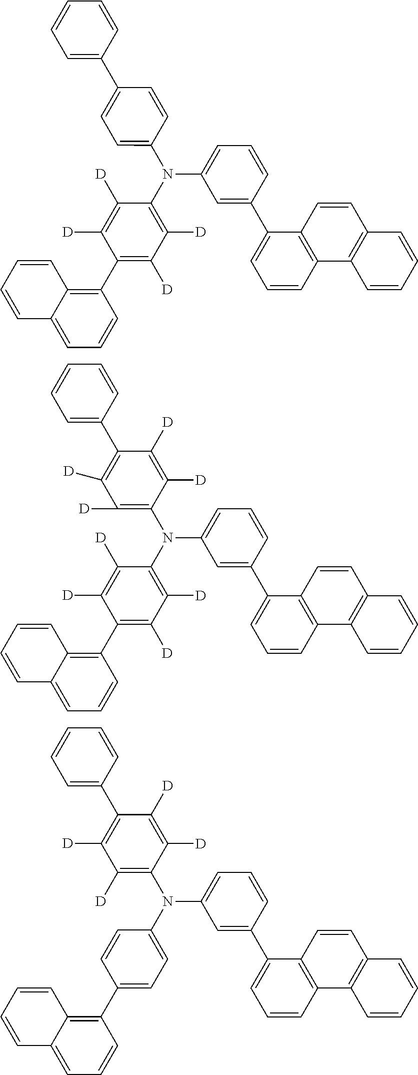

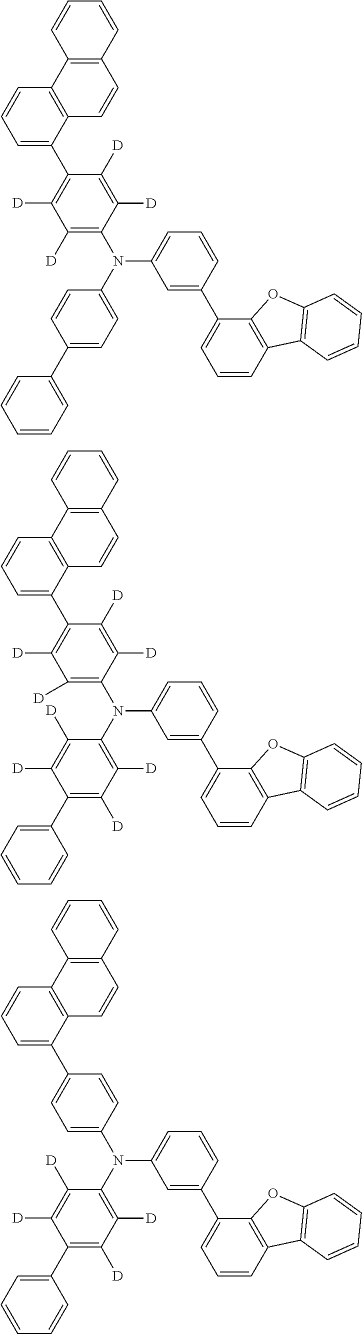

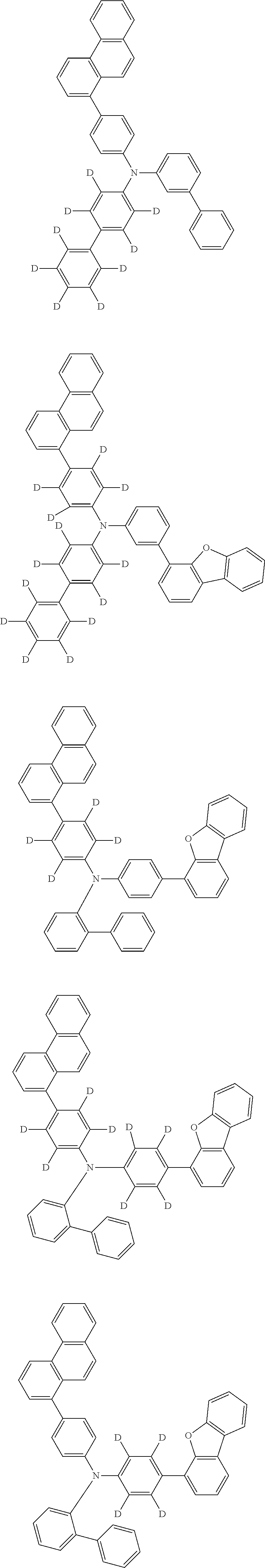

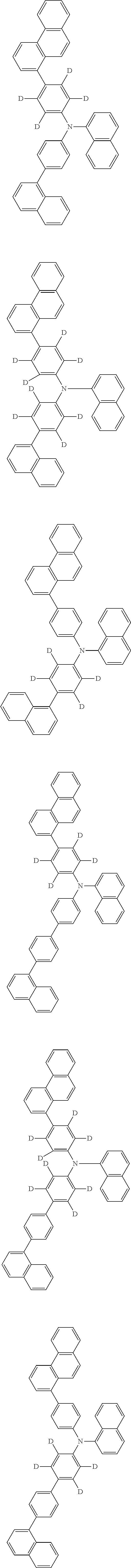

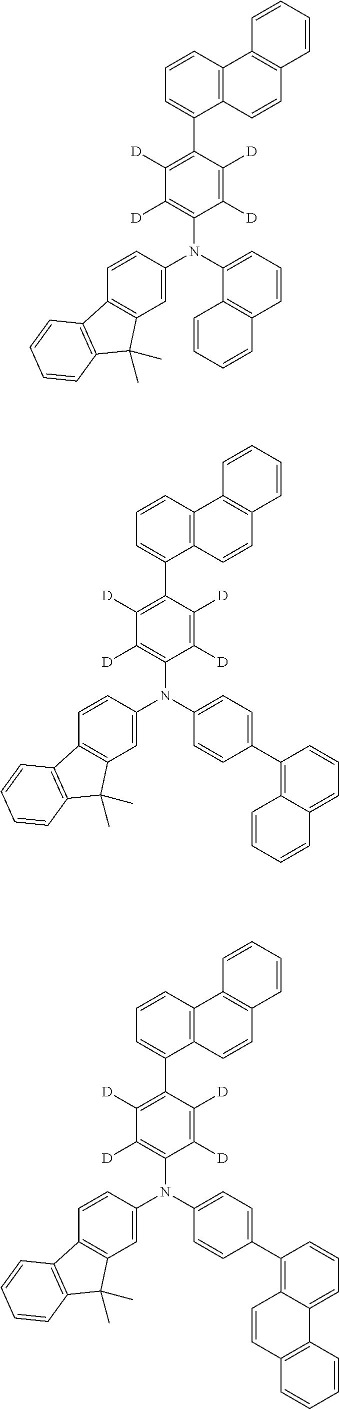 [SMILES: [2H]c1c([2H])c(N(c2ccc3c(c2)C(C)(C)c2ccccc2-3)c2cccc3ccccc23)c([2H])c([2H])c1-c1cccc2c1ccc1ccccc12 ; [2H]c1c([2H])c(N(c2ccc(-c3cccc4ccccc34)cc2)c2ccc3c(c2)C(C)(C)c2ccccc2-3)c([2H])c([2H])c1-c1cccc2c1ccc1ccccc12 ; [2H]c1c([2H])c(N(c2ccc(-c3cccc4c3ccc3ccccc34)cc2)c2ccc3c(c2)C(C)(C)c2ccccc2-3)c([2H])c([2H])c1-c1cccc2c1ccc1ccccc12]

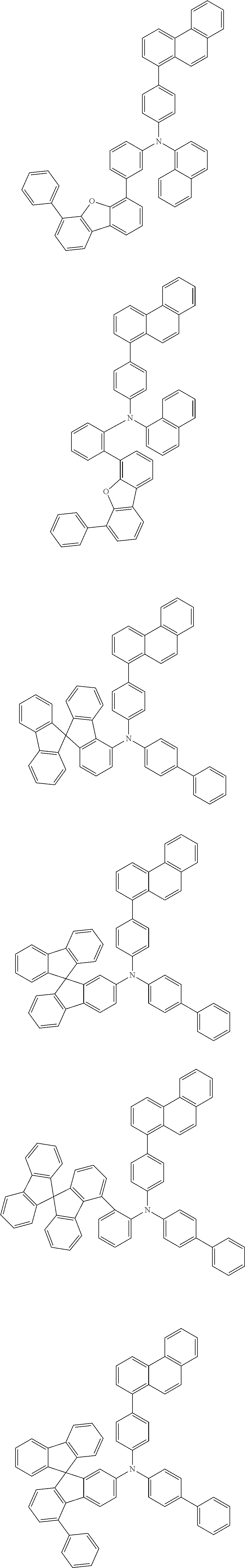

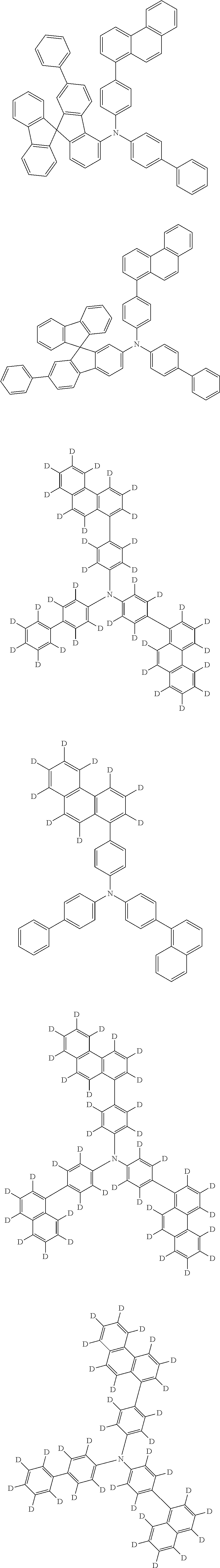

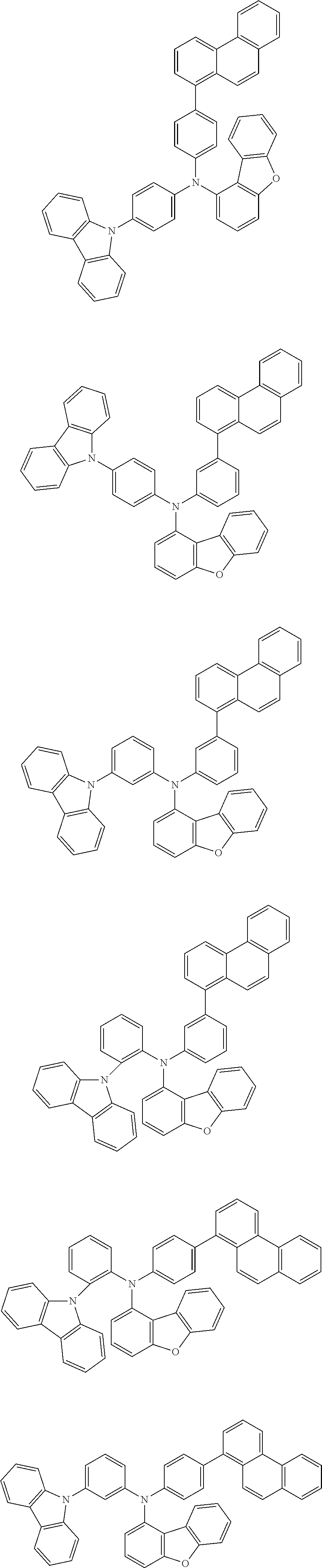

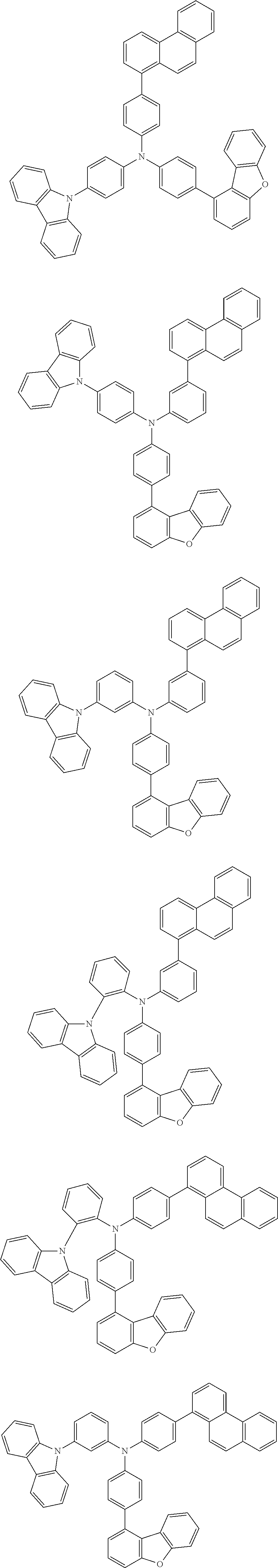

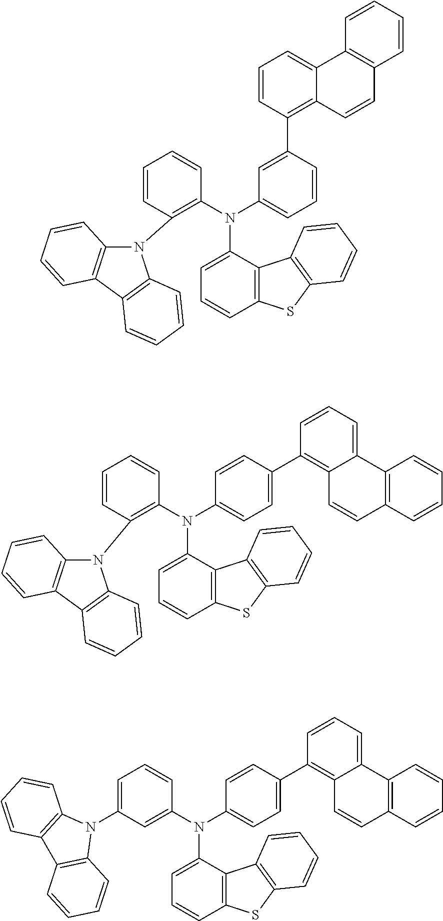 [SMILES: c1cc(-c2cccc3c2ccc2ccccc23)cc(N(c2ccccc2-n2c3ccccc3c3ccccc32)c2cccc3sc4ccccc4c23)c1 ; c1ccc(-n2c3ccccc3c3ccccc32)c(N(c2ccc(-c3cccc4c3ccc3ccccc34)cc2)c2cccc3sc4ccccc4c23)c1 ; c1cc(N(c2ccc(-c3cccc4c3ccc3ccccc34)cc2)c2cccc3sc4ccccc4c23)cc(-n2c3ccccc3c3ccccc32)c1]

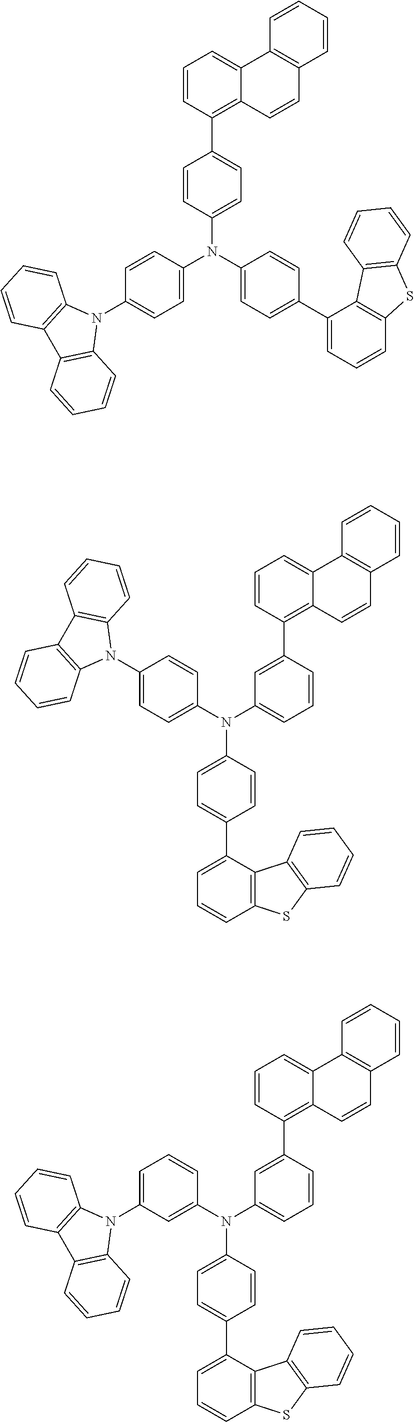 [SMILES: c1ccc2c(c1)ccc1c(-c3ccc(N(c4ccc(-c5cccc6sc7ccccc7c56)cc4)c4ccc(-n5c6ccccc6c6ccccc65)cc4)cc3)cccc12 ; c1cc(-c2cccc3c2ccc2ccccc23)cc(N(c2ccc(-c3cccc4sc5ccccc5c34)cc2)c2ccc(-n3c4ccccc4c4ccccc43)cc2)c1 ; c1cc(-c2cccc3c2ccc2ccccc23)cc(N(c2ccc(-c3cccc4sc5ccccc5c34)cc2)c2cccc(-n3c4ccccc4c4ccccc43)c2)c1]

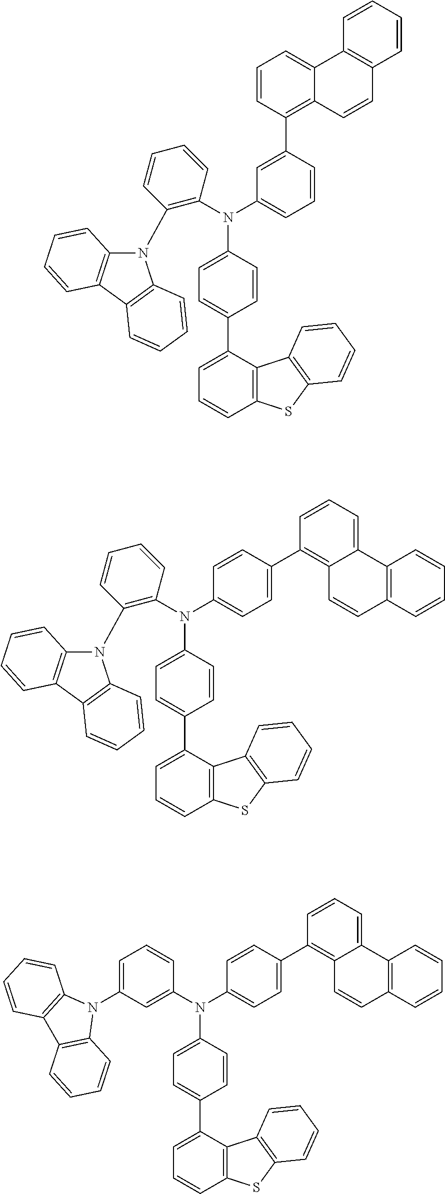 [SMILES: c1cc(-c2cccc3c2ccc2ccccc23)cc(N(c2ccc(-c3cccc4sc5ccccc5c34)cc2)c2ccccc2-n2c3ccccc3c3ccccc32)c1 ; c1ccc(-n2c3ccccc3c3ccccc32)c(N(c2ccc(-c3cccc4c3ccc3ccccc34)cc2)c2ccc(-c3cccc4sc5ccccc5c34)cc2)c1 ; c1cc(N(c2ccc(-c3cccc4c3ccc3ccccc34)cc2)c2ccc(-c3cccc4sc5ccccc5c34)cc2)cc(-n2c3ccccc3c3ccccc32)c1]

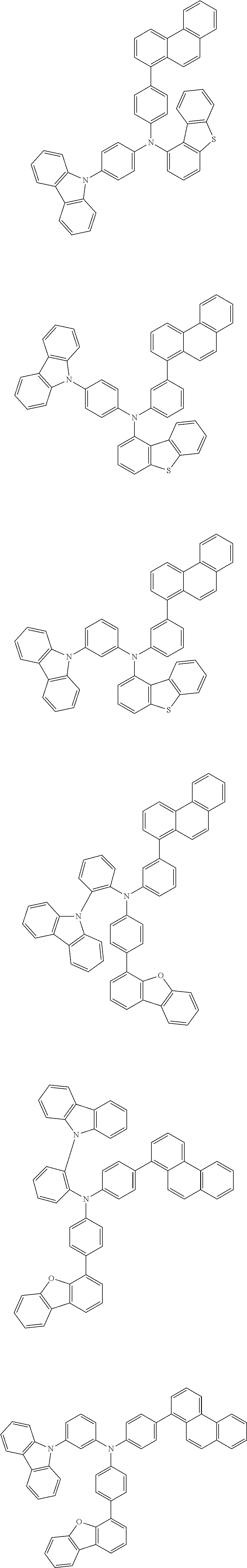

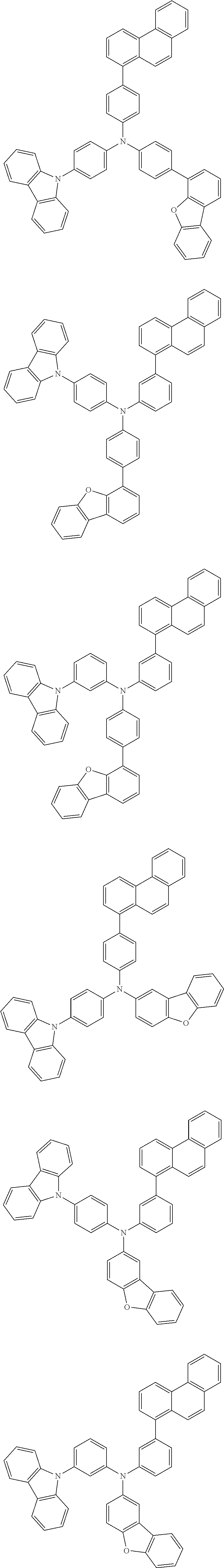

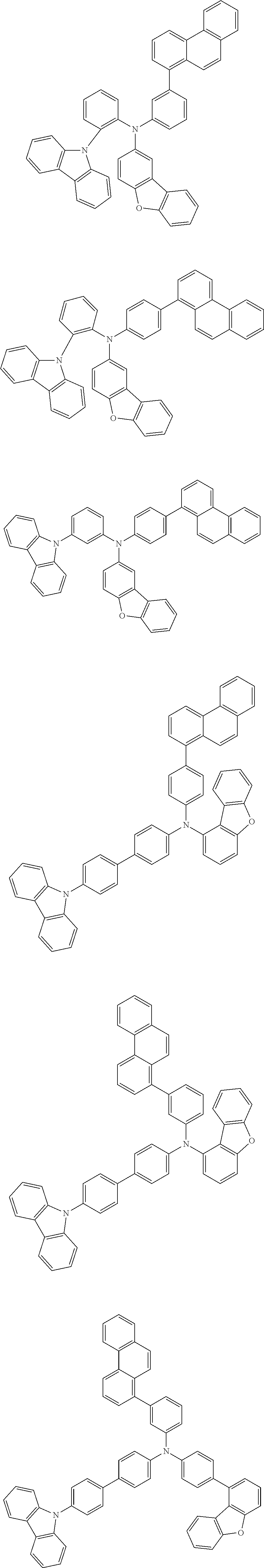

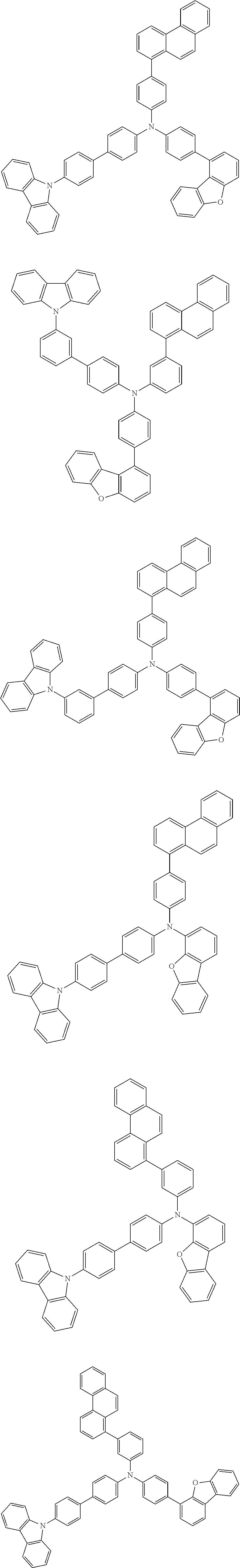

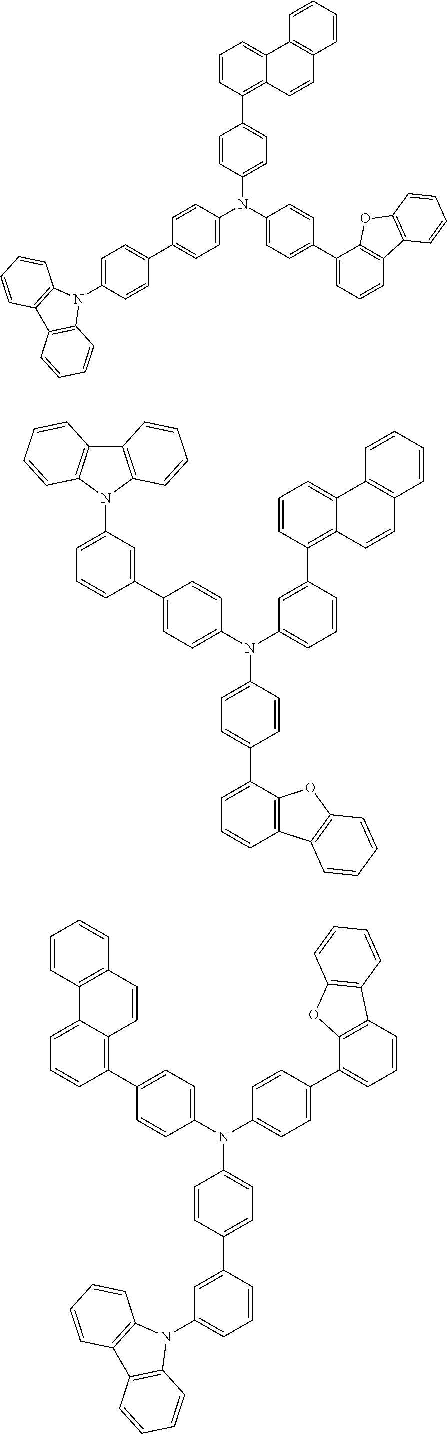

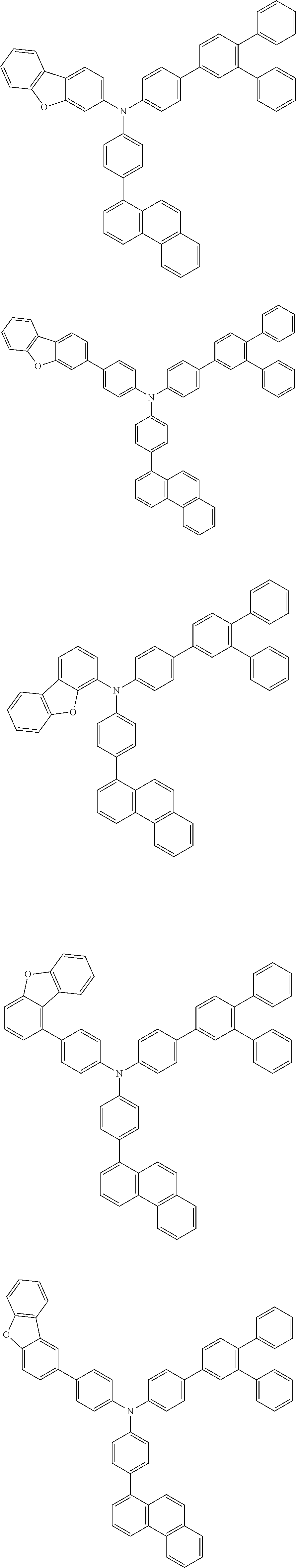

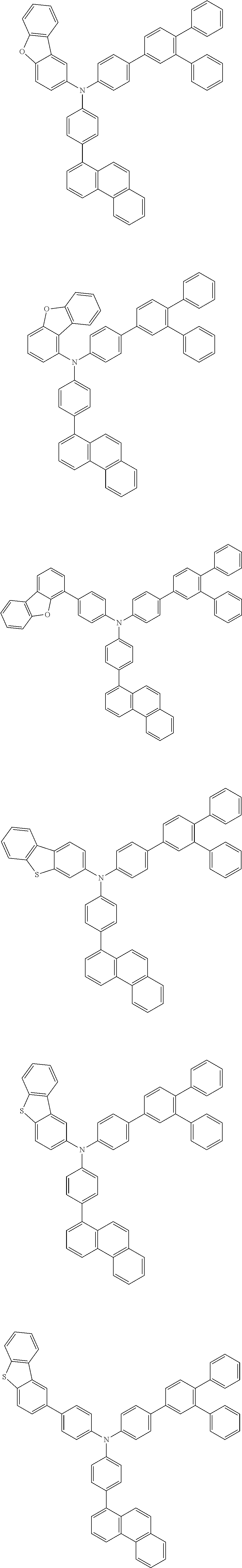

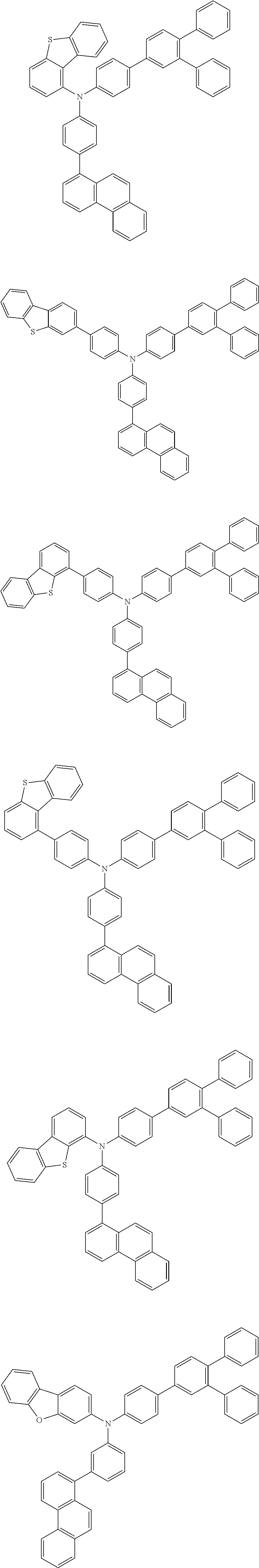

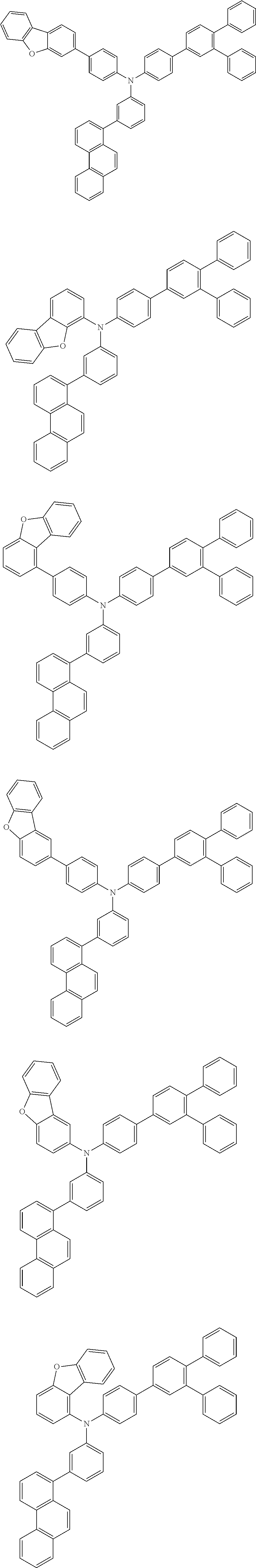

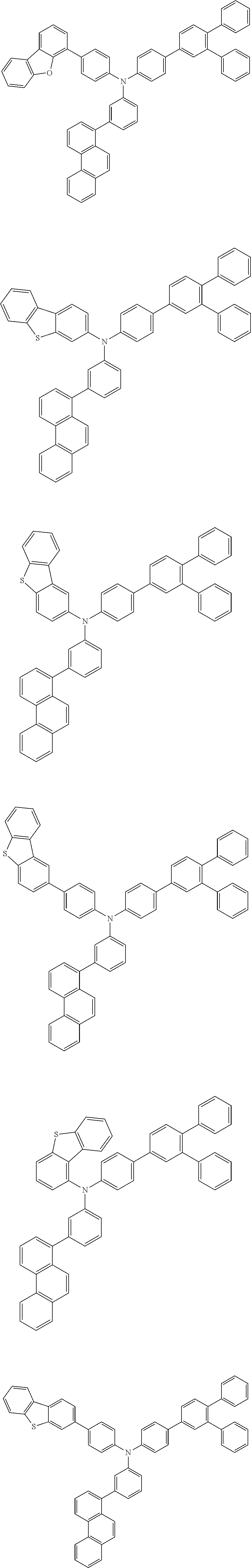

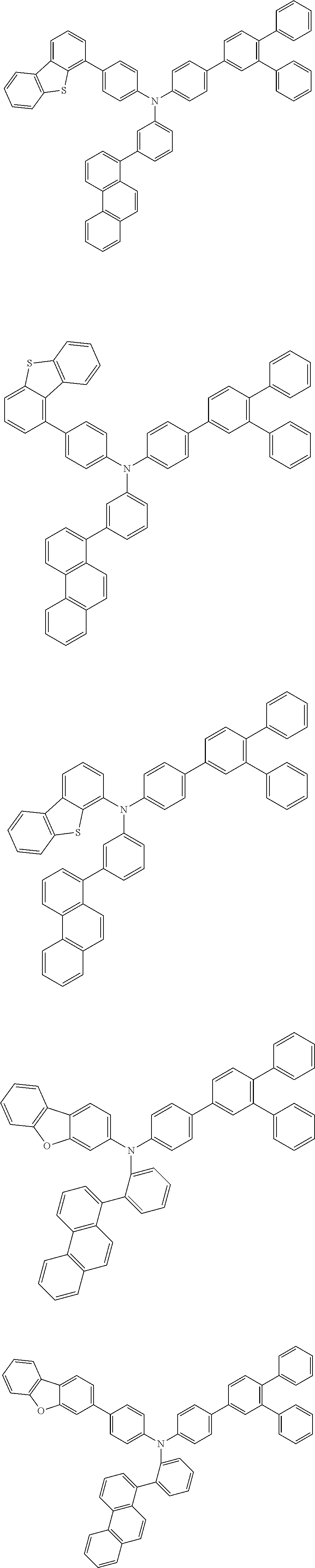

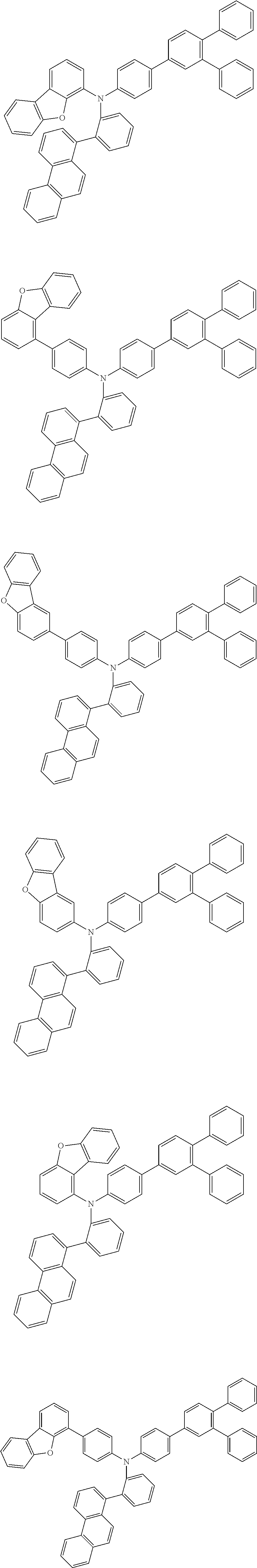

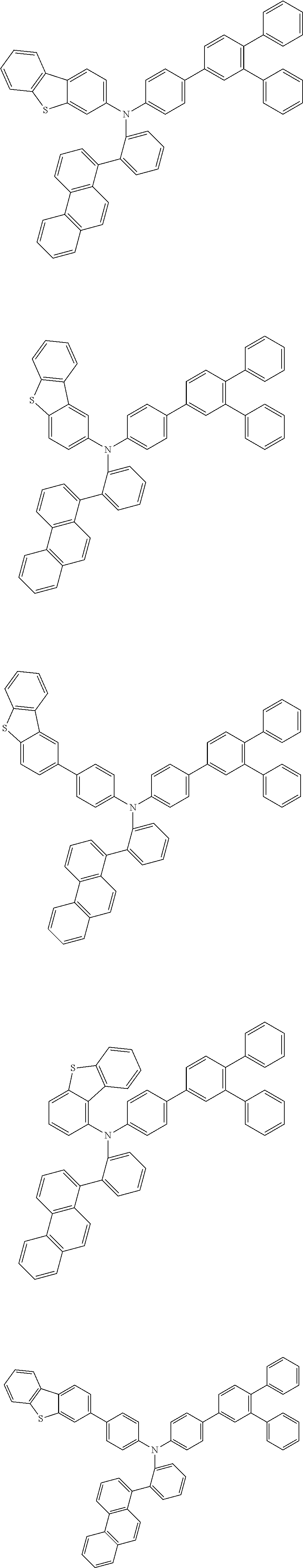 [SMILES: c1ccc(-c2ccc(-c3ccc(N(c4ccc5c(c4)sc4ccccc45)c4ccccc4-c4cccc5c4ccc4ccccc45)cc3)cc2-c2ccccc2)cc1 ; c1ccc(-c2ccc(-c3ccc(N(c4ccc5sc6ccccc6c5c4)c4ccccc4-c4cccc5c4ccc4ccccc45)cc3)cc2-c2ccccc2)cc1 ; c1ccc(-c2ccc(-c3ccc(N(c4ccc(-c5ccc6sc7ccccc7c6c5)cc4)c4ccccc4-c4cccc5c4ccc4ccccc45)cc3)cc2-c2ccccc2)cc1 ; c1ccc(-c2ccc(-c3ccc(N(c4ccccc4-c4cccc5c4ccc4ccccc45)c4cccc5sc6ccccc6c45)cc3)cc2-c2ccccc2)cc1 ; c1ccc(-c2ccc(-c3ccc(N(c4ccc(-c5ccc6c(c5)sc5ccccc56)cc4)c4ccccc4-c4cccc5c4ccc4ccccc45)cc3)cc2-c2ccccc2)cc1]

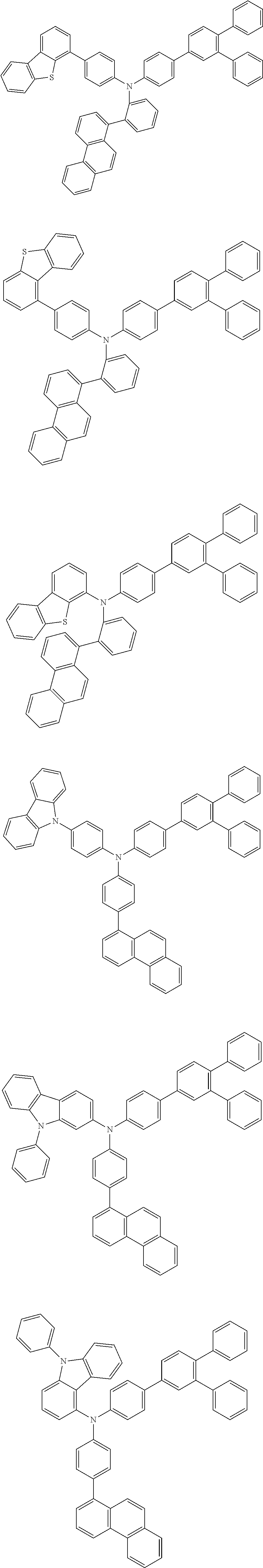

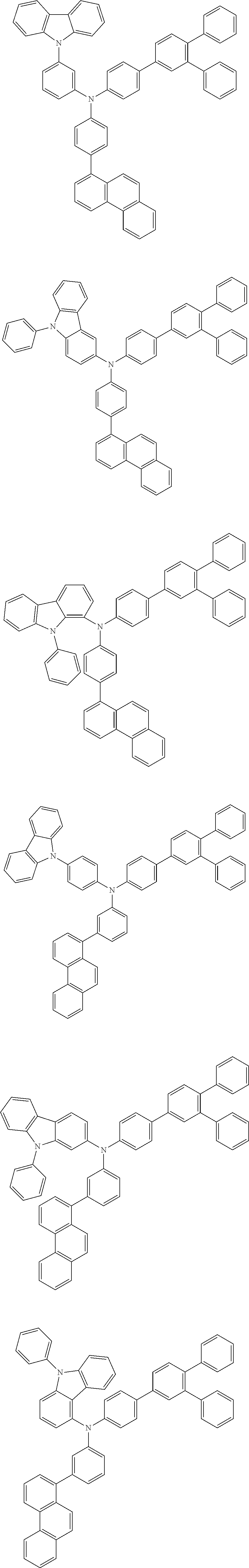

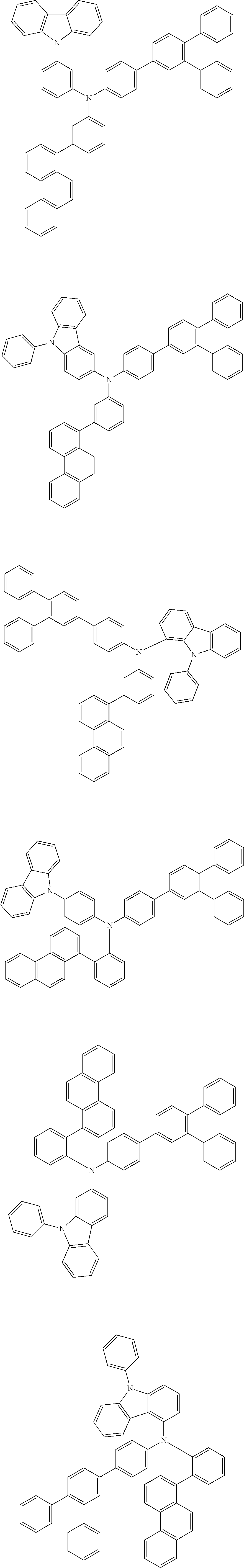

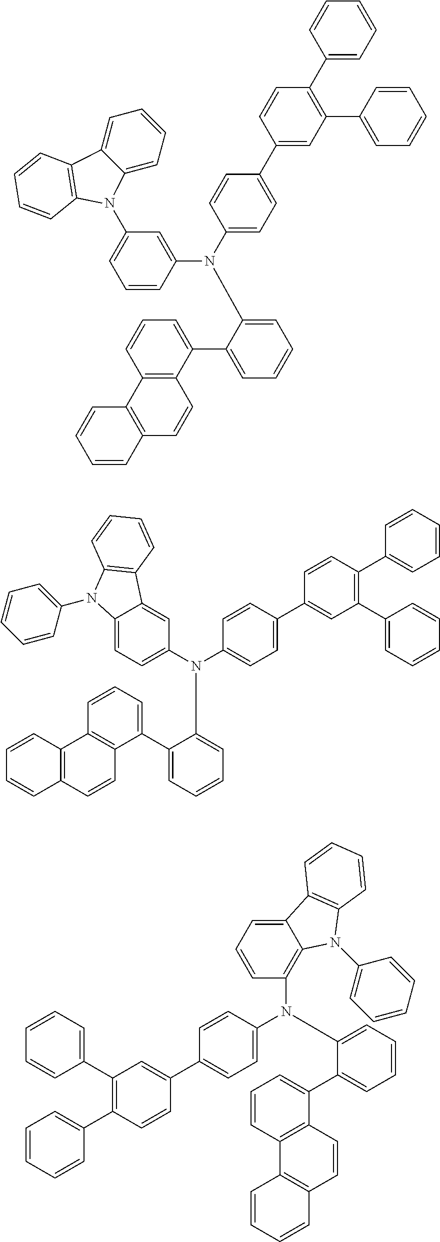 [SMILES: c1ccc(-c2ccc(-c3ccc(N(c4cccc(-n5c6ccccc6c6ccccc65)c4)c4ccccc4-c4cccc5c4ccc4ccccc45)cc3)cc2-c2ccccc2)cc1 ; c1ccc(-c2ccc(-c3ccc(N(c4ccc5c(c4)c4ccccc4n5-c4ccccc4)c4ccccc4-c4cccc5c4ccc4ccccc45)cc3)cc2-c2ccccc2)cc1 ; c1ccc(-c2ccc(-c3ccc(N(c4ccccc4-c4cccc5c4ccc4ccccc45)c4cccc5c6ccccc6n(-c6ccccc6)c45)cc3)cc2-c2ccccc2)cc1]

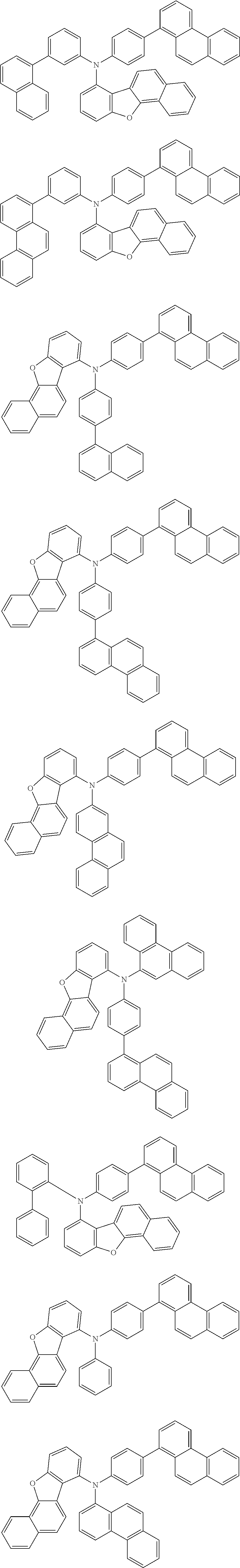

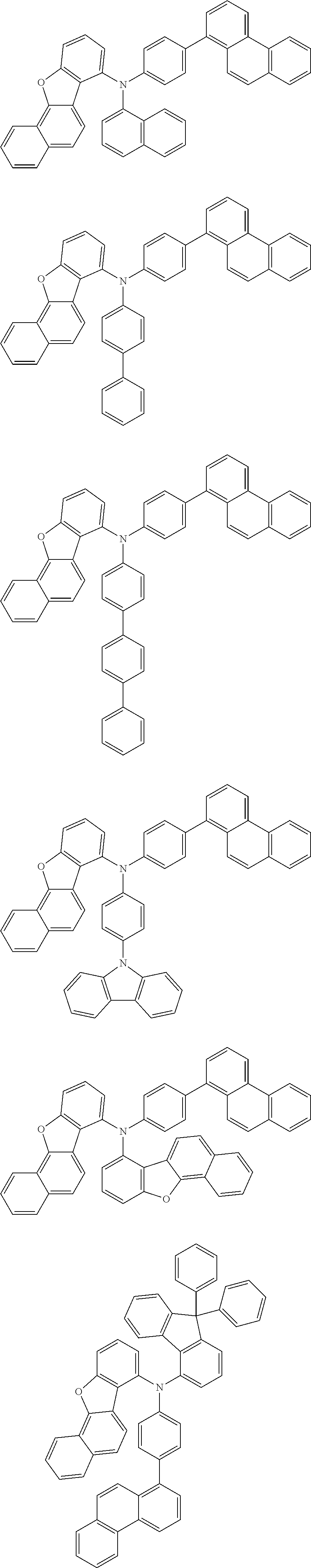

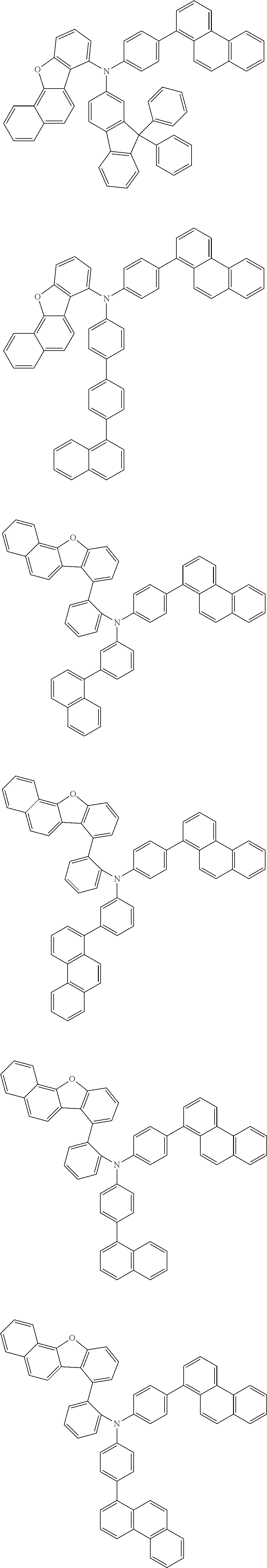

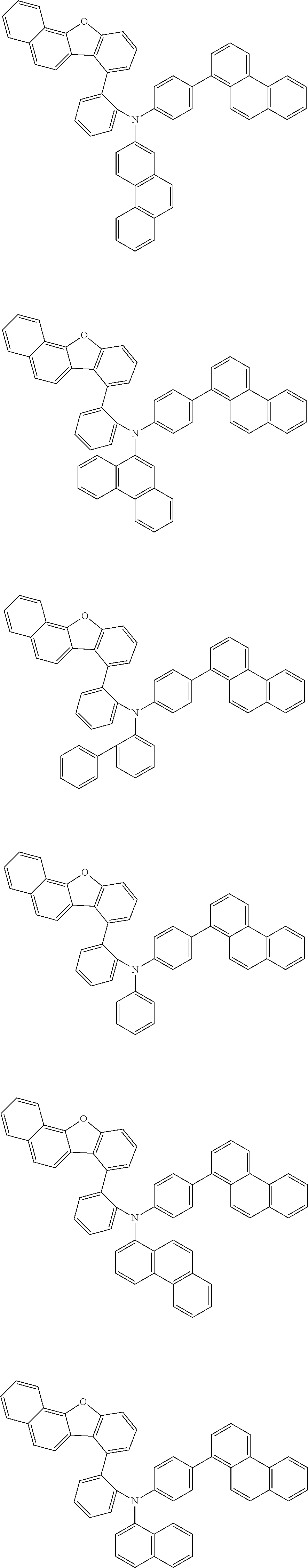

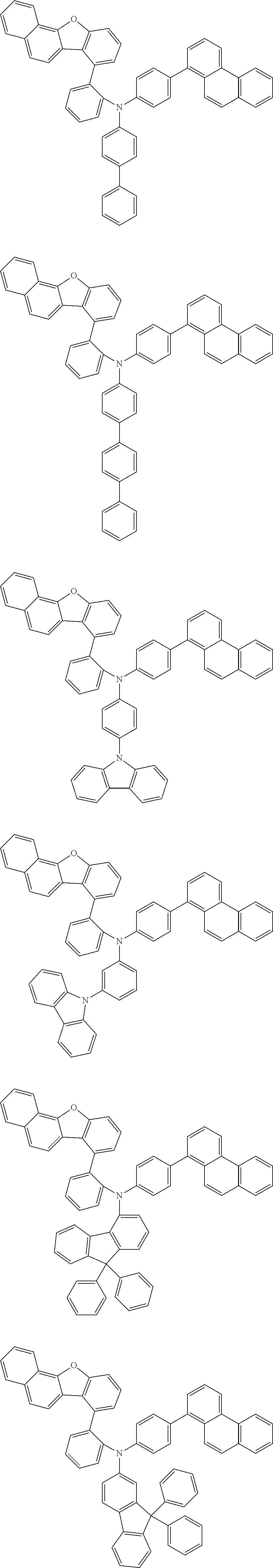

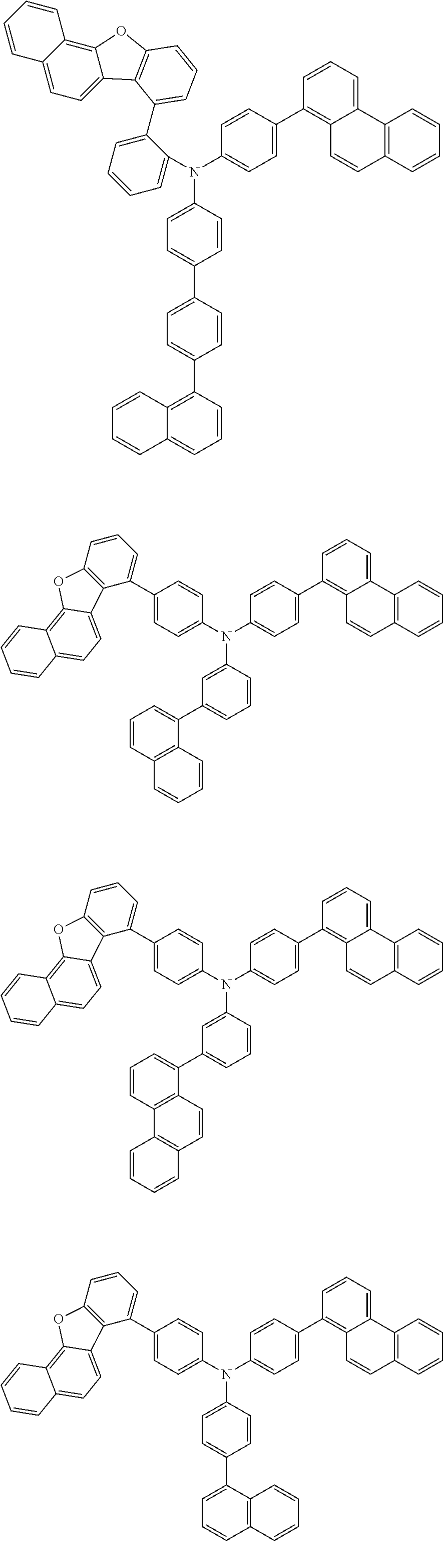 [SMILES: c1ccc(N(c2ccc(-c3ccc(-c4cccc5ccccc45)cc3)cc2)c2ccc(-c3cccc4c3ccc3ccccc34)cc2)c(-c2cccc3oc4c5ccccc5ccc4c23)c1 ; c1cc(-c2cccc3ccccc23)cc(N(c2ccc(-c3cccc4c3ccc3ccccc34)cc2)c2ccc(-c3cccc4oc5c6ccccc6ccc5c34)cc2)c1 ; c1cc(-c2cccc3c2ccc2ccccc23)cc(N(c2ccc(-c3cccc4c3ccc3ccccc34)cc2)c2ccc(-c3cccc4oc5c6ccccc6ccc5c34)cc2)c1 ; c1ccc2c(-c3ccc(N(c4ccc(-c5cccc6c5ccc5ccccc56)cc4)c4ccc(-c5cccc6oc7c8ccccc8ccc7c56)cc4)cc3)cccc2c1]

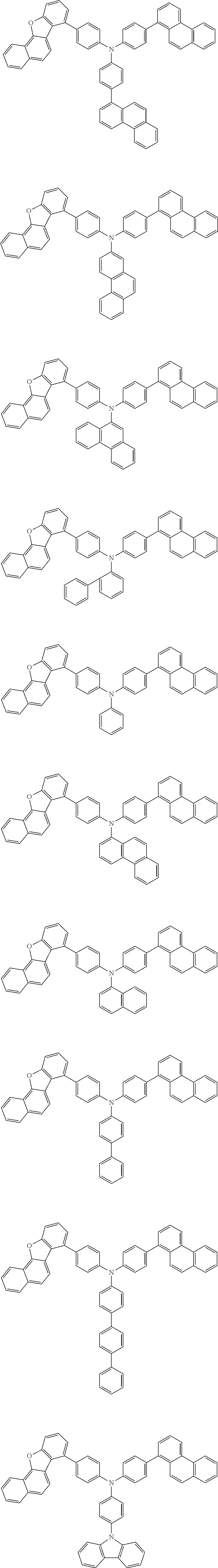

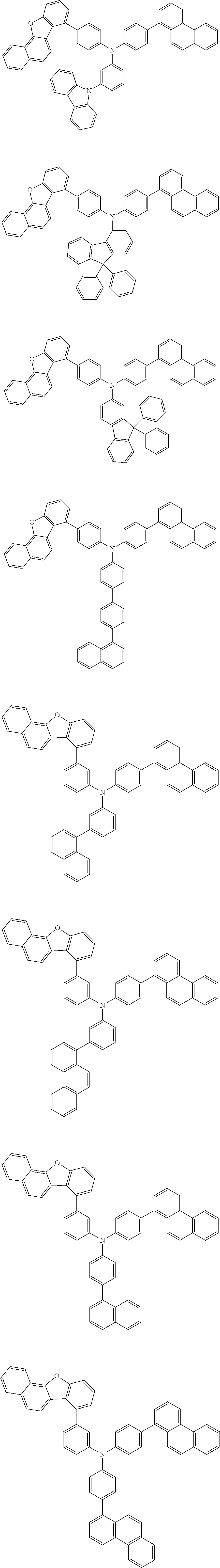

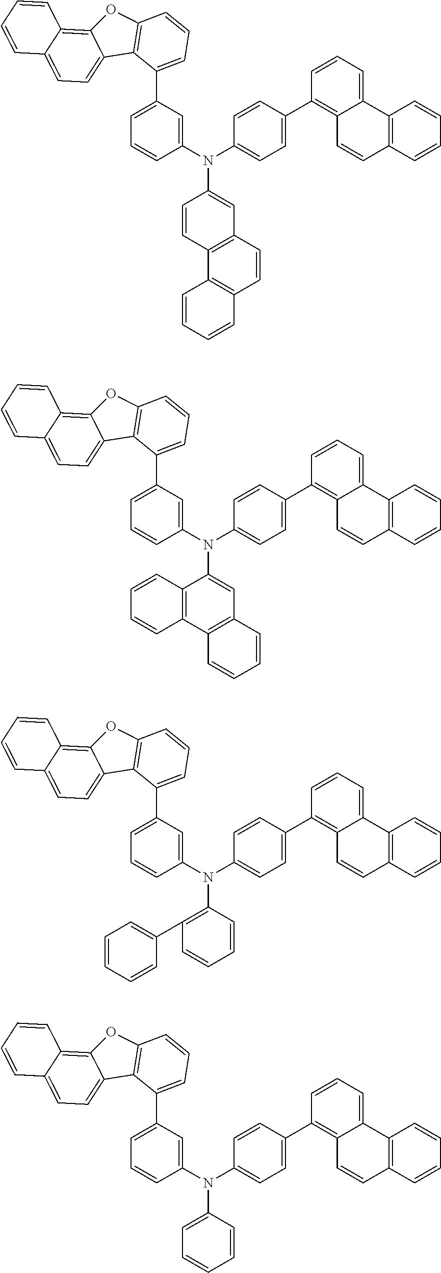 [SMILES: c1cc(-c2cccc3oc4c5ccccc5ccc4c23)cc(N(c2ccc(-c3cccc4c3ccc3ccccc34)cc2)c2ccc3c(ccc4ccccc43)c2)c1 ; c1cc(-c2cccc3oc4c5ccccc5ccc4c23)cc(N(c2ccc(-c3cccc4c3ccc3ccccc34)cc2)c2cc3ccccc3c3ccccc23)c1 ; c1ccc(-c2ccccc2N(c2ccc(-c3cccc4c3ccc3ccccc34)cc2)c2cccc(-c3cccc4oc5c6ccccc6ccc5c34)c2)cc1 ; c1ccc(N(c2ccc(-c3cccc4c3ccc3ccccc34)cc2)c2cccc(-c3cccc4oc5c6ccccc6ccc5c34)c2)cc1]

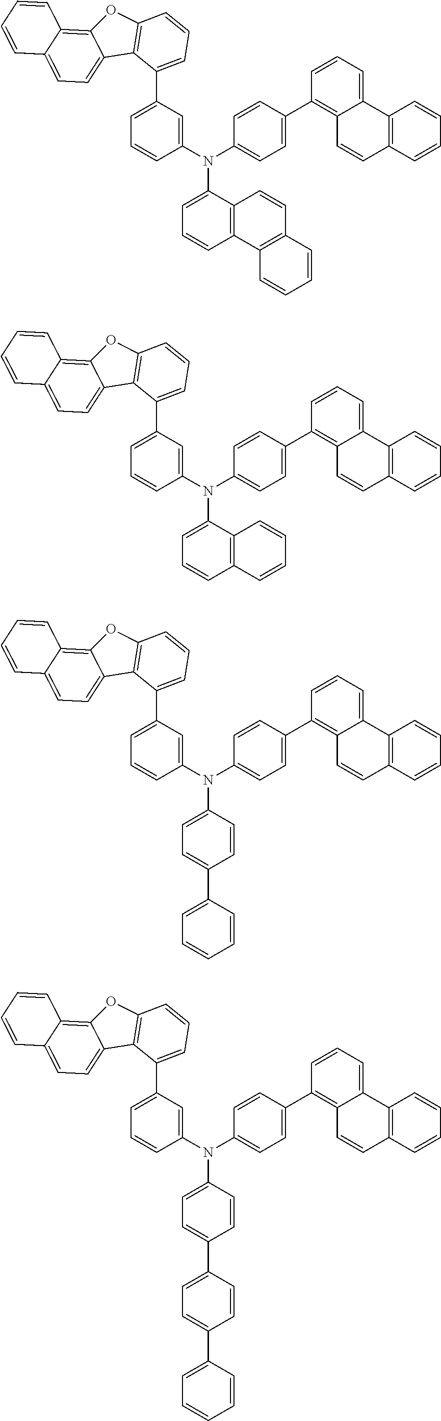 [SMILES: c1cc(-c2cccc3oc4c5ccccc5ccc4c23)cc(N(c2ccc(-c3cccc4c3ccc3ccccc34)cc2)c2cccc3c2ccc2ccccc23)c1 ; c1cc(-c2cccc3oc4c5ccccc5ccc4c23)cc(N(c2ccc(-c3cccc4c3ccc3ccccc34)cc2)c2cccc3ccccc23)c1 ; c1ccc(-c2ccc(N(c3ccc(-c4cccc5c4ccc4ccccc45)cc3)c3cccc(-c4cccc5oc6c7ccccc7ccc6c45)c3)cc2)cc1 ; c1ccc(-c2ccc(-c3ccc(N(c4ccc(-c5cccc6c5ccc5ccccc56)cc4)c4cccc(-c5cccc6oc7c8ccccc8ccc7c56)c4)cc3)cc2)cc1]

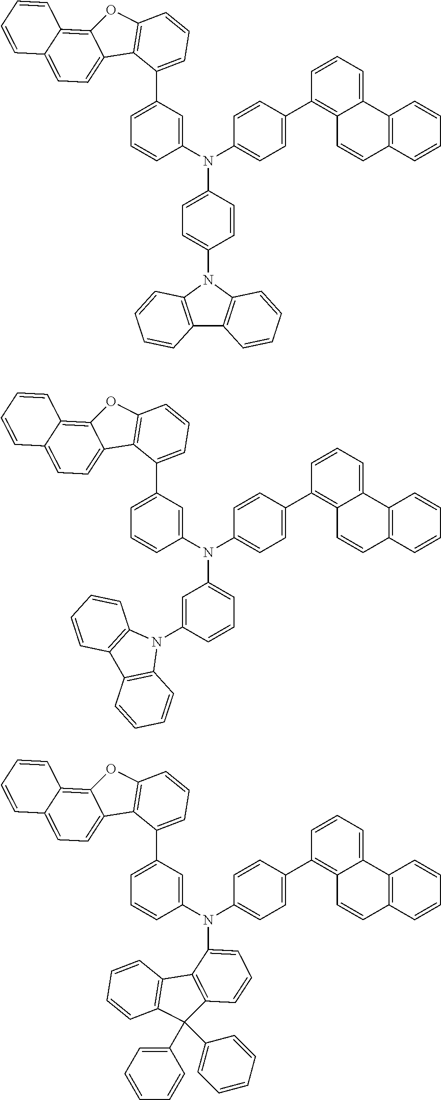 [SMILES: c1cc(-c2cccc3oc4c5ccccc5ccc4c23)cc(N(c2ccc(-c3cccc4c3ccc3ccccc34)cc2)c2ccc(-n3c4ccccc4c4ccccc43)cc2)c1 ; c1cc(-c2cccc3oc4c5ccccc5ccc4c23)cc(N(c2ccc(-c3cccc4c3ccc3ccccc34)cc2)c2cccc(-n3c4ccccc4c4ccccc43)c2)c1 ; c1ccc(C2(c3ccccc3)c3ccccc3-c3c(N(c4ccc(-c5cccc6c5ccc5ccccc56)cc4)c4cccc(-c5cccc6oc7c8ccccc8ccc7c56)c4)cccc32)cc1]

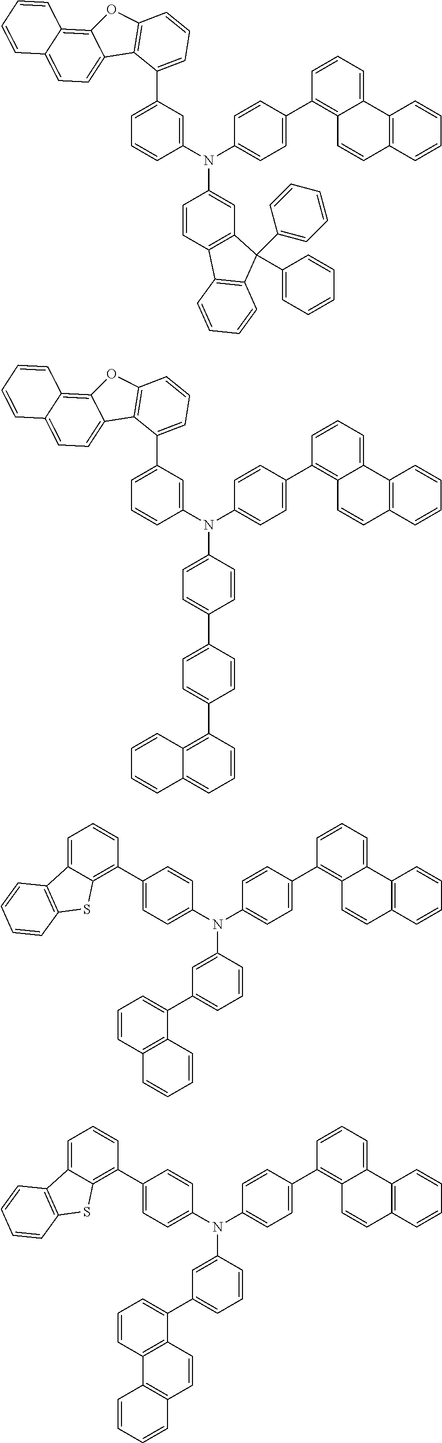 [SMILES: c1ccc(C2(c3ccccc3)c3ccccc3-c3ccc(N(c4ccc(-c5cccc6c5ccc5ccccc56)cc4)c4cccc(-c5cccc6oc7c8ccccc8ccc7c56)c4)cc32)cc1 ; c1cc(-c2cccc3oc4c5ccccc5ccc4c23)cc(N(c2ccc(-c3ccc(-c4cccc5ccccc45)cc3)cc2)c2ccc(-c3cccc4c3ccc3ccccc34)cc2)c1 ; c1cc(-c2cccc3ccccc23)cc(N(c2ccc(-c3cccc4c3ccc3ccccc34)cc2)c2ccc(-c3cccc4c3sc3ccccc34)cc2)c1 ; c1cc(-c2cccc3c2ccc2ccccc23)cc(N(c2ccc(-c3cccc4c3ccc3ccccc34)cc2)c2ccc(-c3cccc4c3sc3ccccc34)cc2)c1]

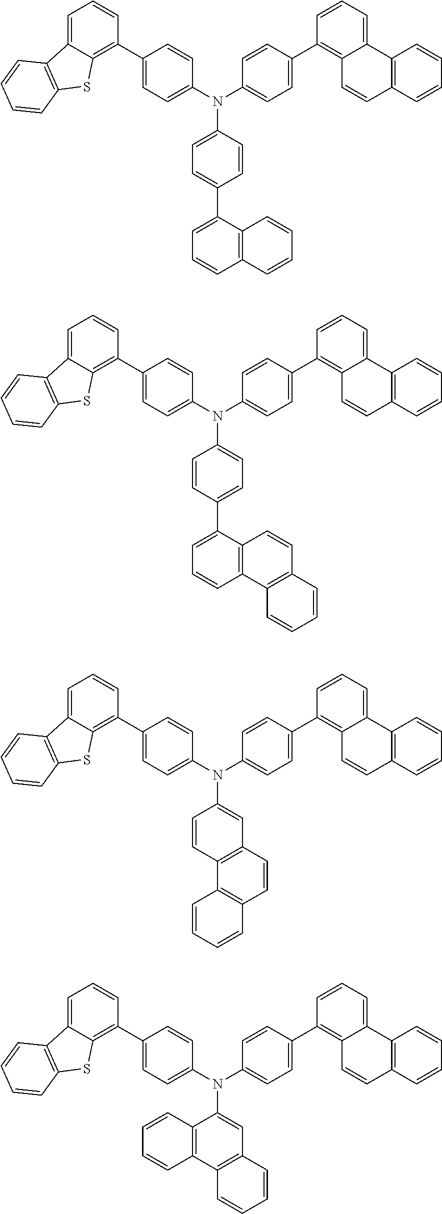 [SMILES: c1ccc2c(-c3ccc(N(c4ccc(-c5cccc6c5ccc5ccccc56)cc4)c4ccc(-c5cccc6c5sc5ccccc56)cc4)cc3)cccc2c1 ; c1ccc2c(c1)ccc1c(-c3ccc(N(c4ccc(-c5cccc6c5ccc5ccccc56)cc4)c4ccc(-c5cccc6c5sc5ccccc56)cc4)cc3)cccc12 ; c1ccc2c(c1)ccc1cc(N(c3ccc(-c4cccc5c4ccc4ccccc45)cc3)c3ccc(-c4cccc5c4sc4ccccc45)cc3)ccc12 ; c1ccc2c(c1)ccc1c(-c3ccc(N(c4ccc(-c5cccc6c5sc5ccccc56)cc4)c4cc5ccccc5c5ccccc45)cc3)cccc12]

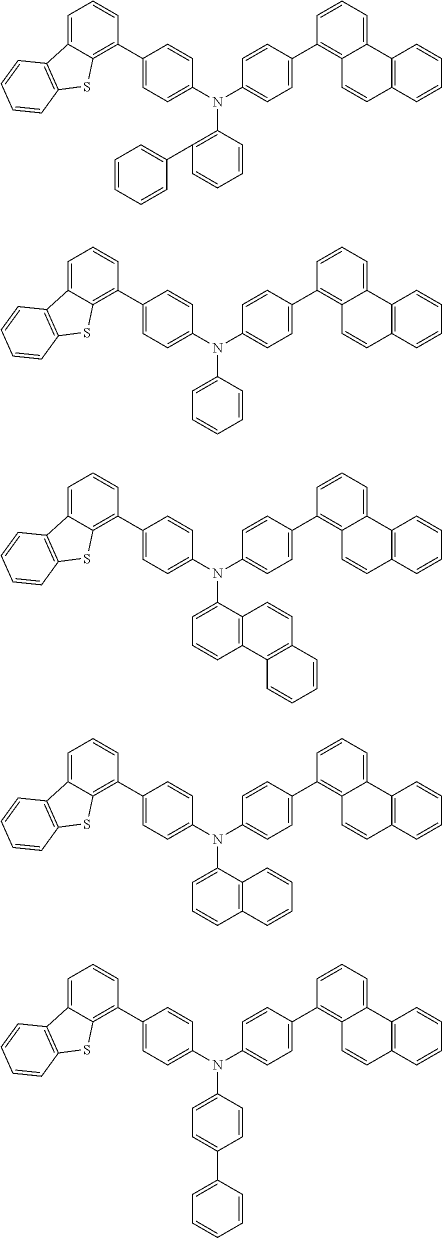 [SMILES: c1ccc(-c2ccccc2N(c2ccc(-c3cccc4c3ccc3ccccc34)cc2)c2ccc(-c3cccc4c3sc3ccccc34)cc2)cc1 ; c1ccc(N(c2ccc(-c3cccc4c3ccc3ccccc34)cc2)c2ccc(-c3cccc4c3sc3ccccc34)cc2)cc1 ; c1ccc2c(c1)ccc1c(-c3ccc(N(c4ccc(-c5cccc6c5sc5ccccc56)cc4)c4cccc5c4ccc4ccccc45)cc3)cccc12 ; c1ccc2c(N(c3ccc(-c4cccc5c4ccc4ccccc45)cc3)c3ccc(-c4cccc5c4sc4ccccc45)cc3)cccc2c1 ; c1ccc(-c2ccc(N(c3ccc(-c4cccc5c4ccc4ccccc45)cc3)c3ccc(-c4cccc5c4sc4ccccc45)cc3)cc2)cc1]

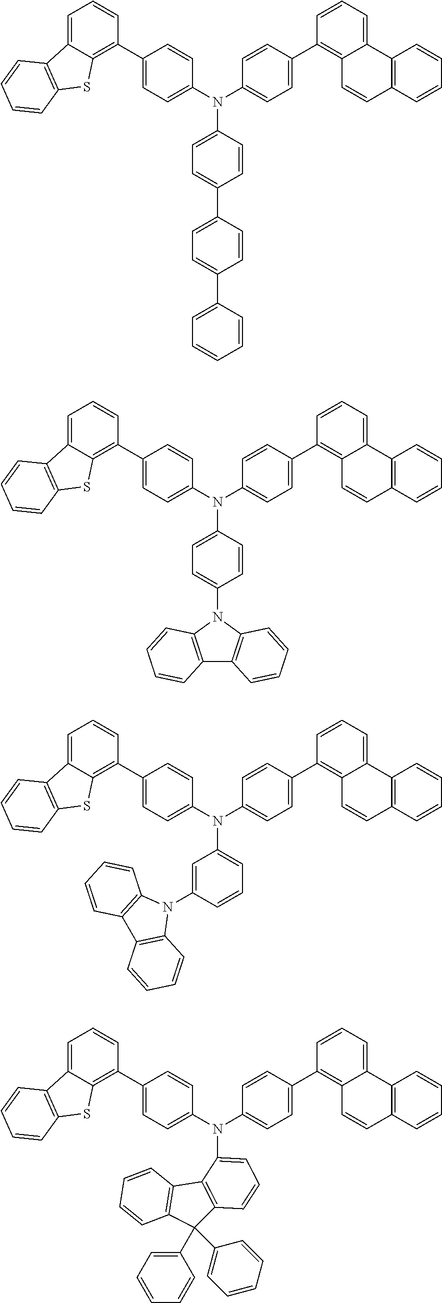 [SMILES: c1ccc(-c2ccc(-c3ccc(N(c4ccc(-c5cccc6c5ccc5ccccc56)cc4)c4ccc(-c5cccc6c5sc5ccccc56)cc4)cc3)cc2)cc1 ; c1ccc2c(c1)ccc1c(-c3ccc(N(c4ccc(-c5cccc6c5sc5ccccc56)cc4)c4ccc(-n5c6ccccc6c6ccccc65)cc4)cc3)cccc12 ; c1cc(N(c2ccc(-c3cccc4c3ccc3ccccc34)cc2)c2ccc(-c3cccc4c3sc3ccccc34)cc2)cc(-n2c3ccccc3c3ccccc32)c1 ; c1ccc(C2(c3ccccc3)c3ccccc3-c3c(N(c4ccc(-c5cccc6c5ccc5ccccc56)cc4)c4ccc(-c5cccc6c5sc5ccccc56)cc4)cccc32)cc1]

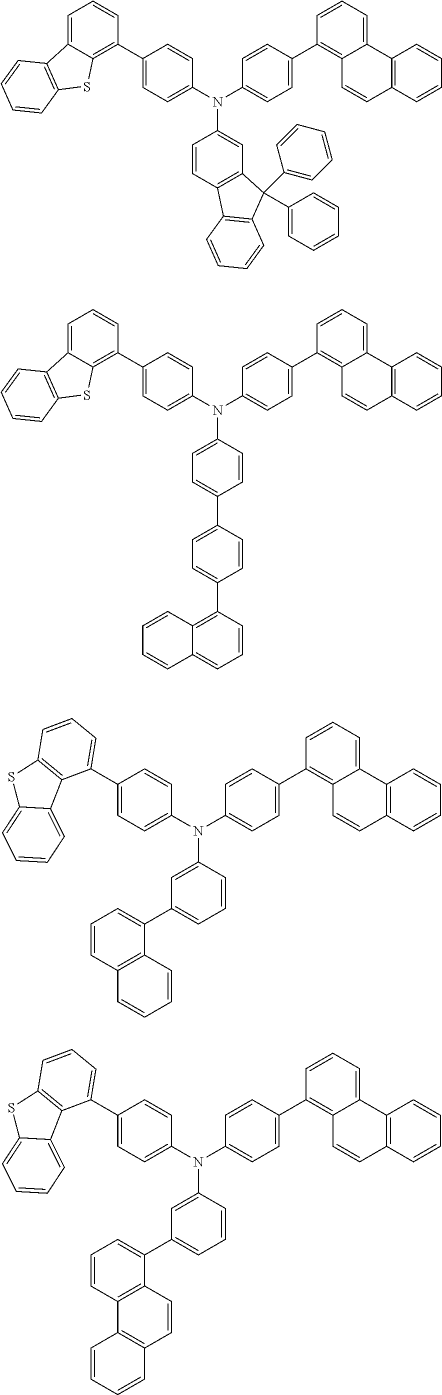 [SMILES: c1ccc(C2(c3ccccc3)c3ccccc3-c3ccc(N(c4ccc(-c5cccc6c5ccc5ccccc56)cc4)c4ccc(-c5cccc6c5sc5ccccc56)cc4)cc32)cc1 ; c1ccc2c(-c3ccc(-c4ccc(N(c5ccc(-c6cccc7c6ccc6ccccc67)cc5)c5ccc(-c6cccc7c6sc6ccccc67)cc5)cc4)cc3)cccc2c1 ; c1cc(-c2cccc3ccccc23)cc(N(c2ccc(-c3cccc4c3ccc3ccccc34)cc2)c2ccc(-c3cccc4sc5ccccc5c34)cc2)c1 ; c1cc(-c2cccc3c2ccc2ccccc23)cc(N(c2ccc(-c3cccc4c3ccc3ccccc34)cc2)c2ccc(-c3cccc4sc5ccccc5c34)cc2)c1]

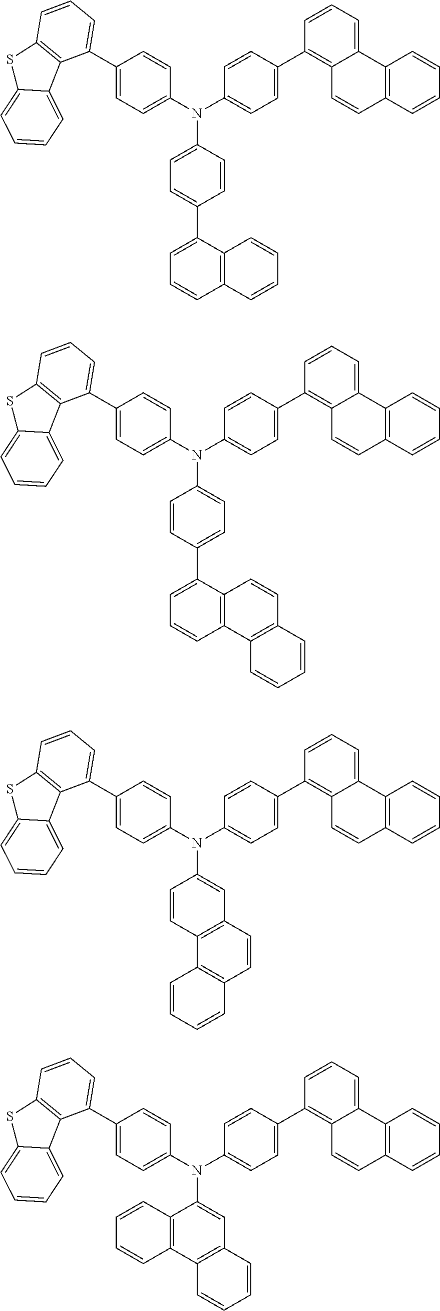 [SMILES: c1ccc2c(-c3ccc(N(c4ccc(-c5cccc6c5ccc5ccccc56)cc4)c4ccc(-c5cccc6sc7ccccc7c56)cc4)cc3)cccc2c1 ; c1ccc2c(c1)ccc1c(-c3ccc(N(c4ccc(-c5cccc6c5ccc5ccccc56)cc4)c4ccc(-c5cccc6sc7ccccc7c56)cc4)cc3)cccc12 ; c1ccc2c(c1)ccc1cc(N(c3ccc(-c4cccc5c4ccc4ccccc45)cc3)c3ccc(-c4cccc5sc6ccccc6c45)cc3)ccc12 ; c1ccc2c(c1)ccc1c(-c3ccc(N(c4ccc(-c5cccc6sc7ccccc7c56)cc4)c4cc5ccccc5c5ccccc45)cc3)cccc12]

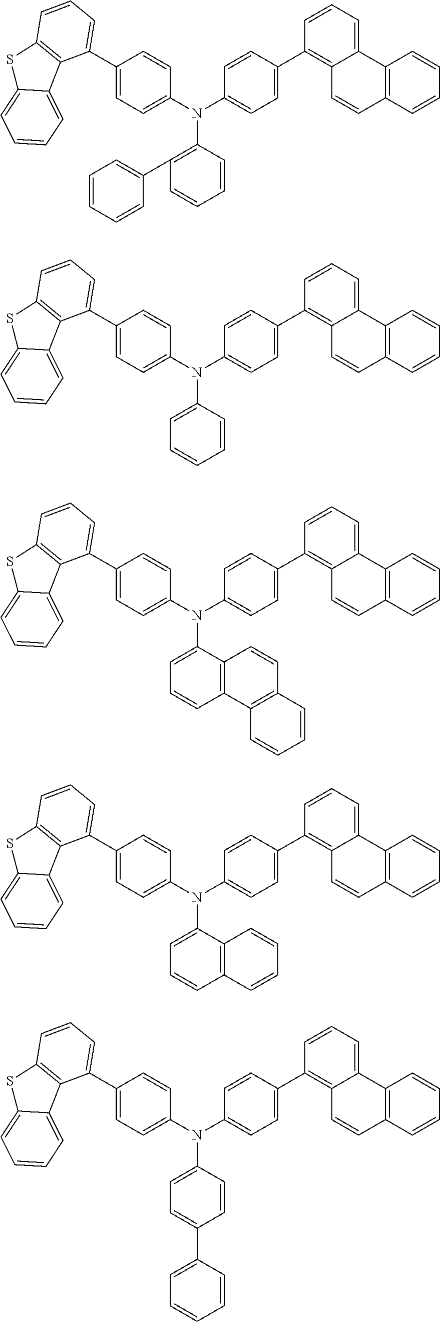 [SMILES: c1ccc(-c2ccccc2N(c2ccc(-c3cccc4c3ccc3ccccc34)cc2)c2ccc(-c3cccc4sc5ccccc5c34)cc2)cc1 ; c1ccc(N(c2ccc(-c3cccc4c3ccc3ccccc34)cc2)c2ccc(-c3cccc4sc5ccccc5c34)cc2)cc1 ; c1ccc2c(c1)ccc1c(-c3ccc(N(c4ccc(-c5cccc6sc7ccccc7c56)cc4)c4cccc5c4ccc4ccccc45)cc3)cccc12 ; c1ccc2c(N(c3ccc(-c4cccc5c4ccc4ccccc45)cc3)c3ccc(-c4cccc5sc6ccccc6c45)cc3)cccc2c1 ; c1ccc(-c2ccc(N(c3ccc(-c4cccc5c4ccc4ccccc45)cc3)c3ccc(-c4cccc5sc6ccccc6c45)cc3)cc2)cc1]

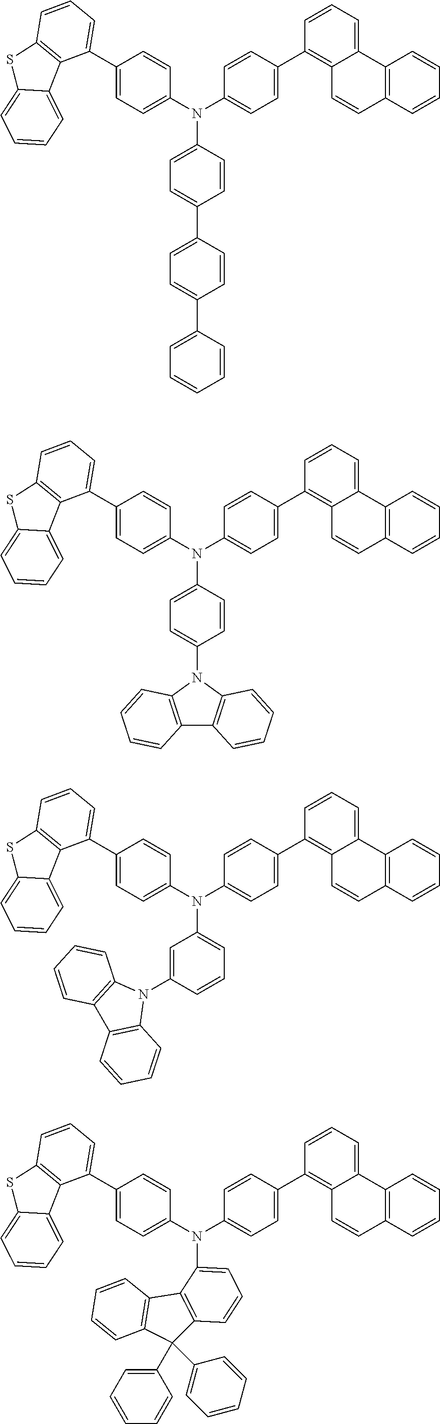 [SMILES: c1ccc(-c2ccc(-c3ccc(N(c4ccc(-c5cccc6c5ccc5ccccc56)cc4)c4ccc(-c5cccc6sc7ccccc7c56)cc4)cc3)cc2)cc1 ; c1ccc2c(c1)ccc1c(-c3ccc(N(c4ccc(-c5cccc6sc7ccccc7c56)cc4)c4ccc(-n5c6ccccc6c6ccccc65)cc4)cc3)cccc12 ; c1cc(N(c2ccc(-c3cccc4c3ccc3ccccc34)cc2)c2ccc(-c3cccc4sc5ccccc5c34)cc2)cc(-n2c3ccccc3c3ccccc32)c1 ; c1ccc(C2(c3ccccc3)c3ccccc3-c3c(N(c4ccc(-c5cccc6c5ccc5ccccc56)cc4)c4ccc(-c5cccc6sc7ccccc7c56)cc4)cccc32)cc1]

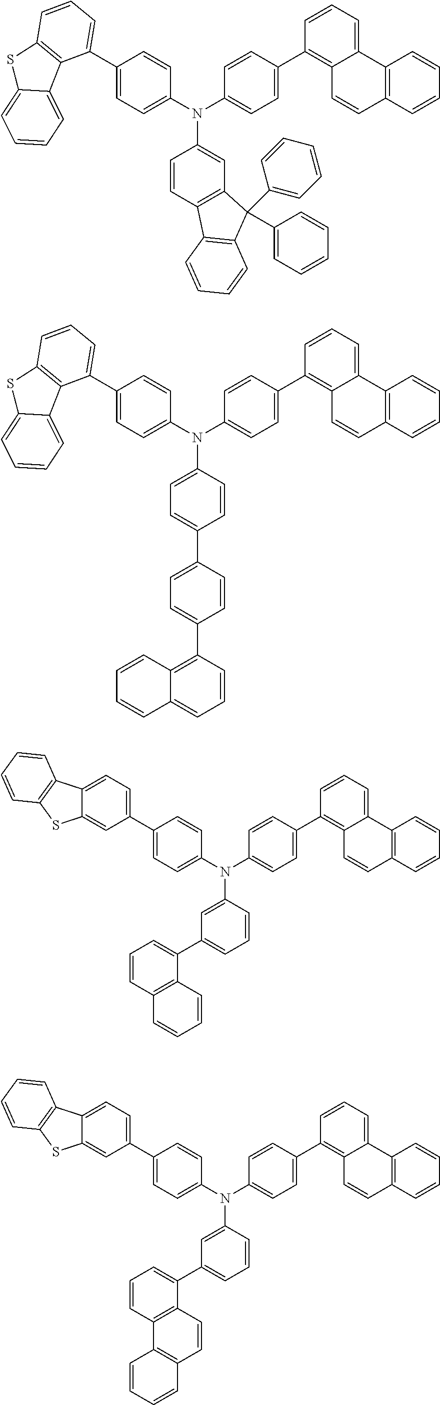 [SMILES: c1ccc(C2(c3ccccc3)c3ccccc3-c3ccc(N(c4ccc(-c5cccc6c5ccc5ccccc56)cc4)c4ccc(-c5cccc6sc7ccccc7c56)cc4)cc32)cc1 ; c1ccc2c(-c3ccc(-c4ccc(N(c5ccc(-c6cccc7c6ccc6ccccc67)cc5)c5ccc(-c6cccc7sc8ccccc8c67)cc5)cc4)cc3)cccc2c1 ; c1cc(-c2cccc3ccccc23)cc(N(c2ccc(-c3ccc4c(c3)sc3ccccc34)cc2)c2ccc(-c3cccc4c3ccc3ccccc34)cc2)c1 ; c1cc(-c2cccc3c2ccc2ccccc23)cc(N(c2ccc(-c3ccc4c(c3)sc3ccccc34)cc2)c2ccc(-c3cccc4c3ccc3ccccc34)cc2)c1]

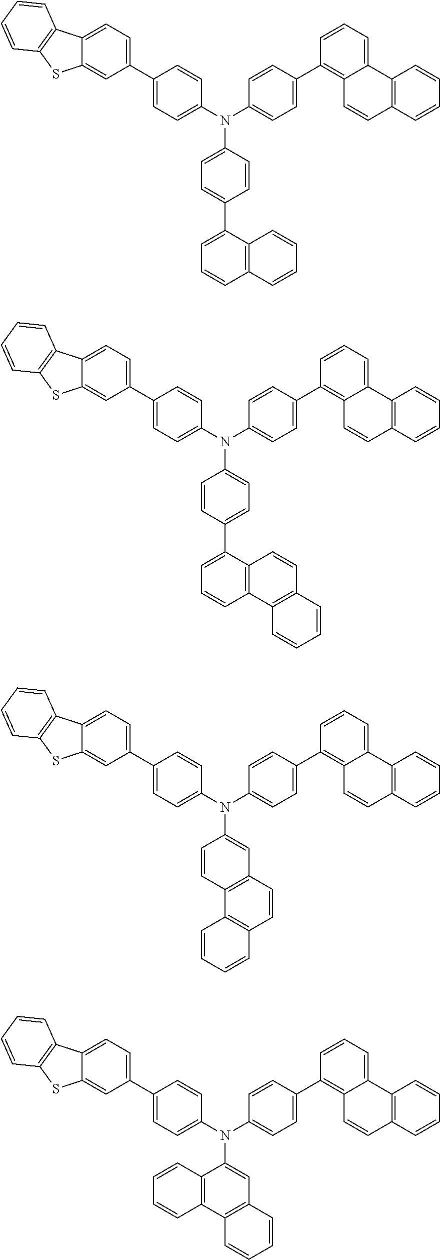 [SMILES: c1ccc2c(-c3ccc(N(c4ccc(-c5ccc6c(c5)sc5ccccc56)cc4)c4ccc(-c5cccc6c5ccc5ccccc56)cc4)cc3)cccc2c1 ; c1ccc2c(c1)ccc1c(-c3ccc(N(c4ccc(-c5ccc6c(c5)sc5ccccc56)cc4)c4ccc(-c5cccc6c5ccc5ccccc56)cc4)cc3)cccc12 ; c1ccc2c(c1)ccc1cc(N(c3ccc(-c4ccc5c(c4)sc4ccccc45)cc3)c3ccc(-c4cccc5c4ccc4ccccc45)cc3)ccc12 ; c1ccc2c(c1)ccc1c(-c3ccc(N(c4ccc(-c5ccc6c(c5)sc5ccccc56)cc4)c4cc5ccccc5c5ccccc45)cc3)cccc12]

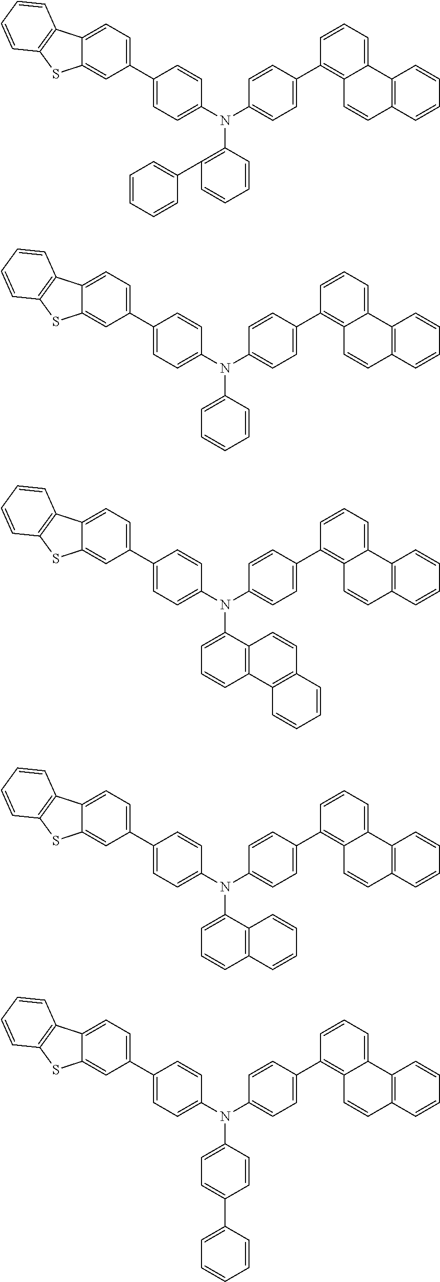 [SMILES: c1ccc(-c2ccccc2N(c2ccc(-c3ccc4c(c3)sc3ccccc34)cc2)c2ccc(-c3cccc4c3ccc3ccccc34)cc2)cc1 ; c1ccc(N(c2ccc(-c3ccc4c(c3)sc3ccccc34)cc2)c2ccc(-c3cccc4c3ccc3ccccc34)cc2)cc1 ; c1ccc2c(c1)ccc1c(-c3ccc(N(c4ccc(-c5ccc6c(c5)sc5ccccc56)cc4)c4cccc5c4ccc4ccccc45)cc3)cccc12 ; c1ccc2c(N(c3ccc(-c4ccc5c(c4)sc4ccccc45)cc3)c3ccc(-c4cccc5c4ccc4ccccc45)cc3)cccc2c1 ; c1ccc(-c2ccc(N(c3ccc(-c4ccc5c(c4)sc4ccccc45)cc3)c3ccc(-c4cccc5c4ccc4ccccc45)cc3)cc2)cc1]

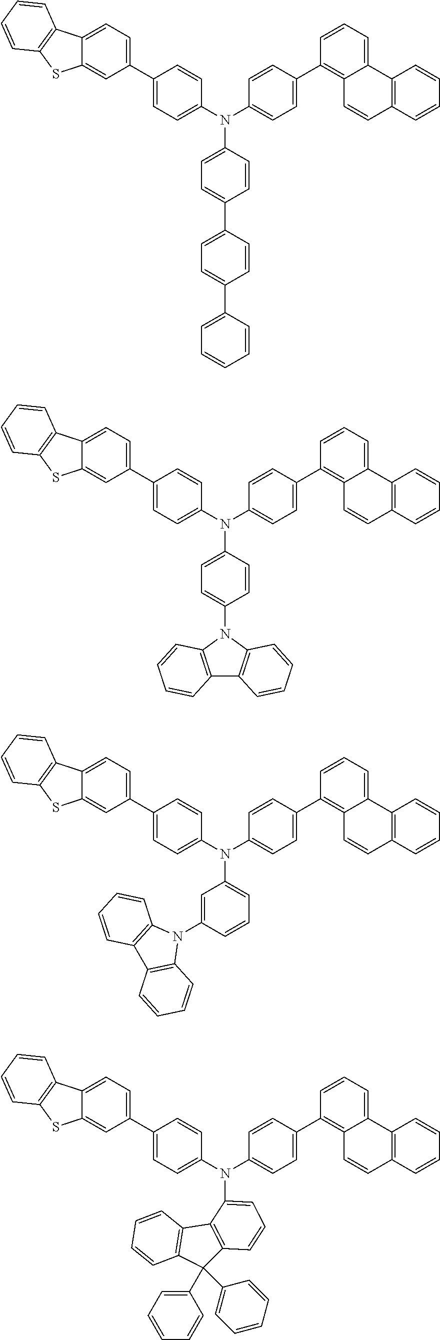 [SMILES: c1ccc(-c2ccc(-c3ccc(N(c4ccc(-c5ccc6c(c5)sc5ccccc56)cc4)c4ccc(-c5cccc6c5ccc5ccccc56)cc4)cc3)cc2)cc1 ; c1ccc2c(c1)ccc1c(-c3ccc(N(c4ccc(-c5ccc6c(c5)sc5ccccc56)cc4)c4ccc(-n5c6ccccc6c6ccccc65)cc4)cc3)cccc12 ; c1cc(N(c2ccc(-c3ccc4c(c3)sc3ccccc34)cc2)c2ccc(-c3cccc4c3ccc3ccccc34)cc2)cc(-n2c3ccccc3c3ccccc32)c1 ; c1ccc(C2(c3ccccc3)c3ccccc3-c3c(N(c4ccc(-c5ccc6c(c5)sc5ccccc56)cc4)c4ccc(-c5cccc6c5ccc5ccccc56)cc4)cccc32)cc1]

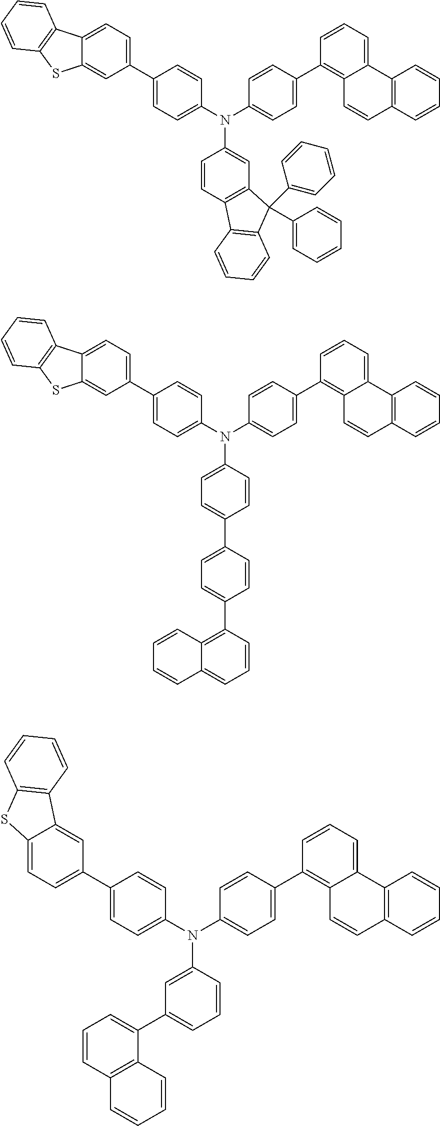 [SMILES: c1ccc(C2(c3ccccc3)c3ccccc3-c3ccc(N(c4ccc(-c5ccc6c(c5)sc5ccccc56)cc4)c4ccc(-c5cccc6c5ccc5ccccc56)cc4)cc32)cc1 ; c1ccc2c(-c3ccc(-c4ccc(N(c5ccc(-c6ccc7c(c6)sc6ccccc67)cc5)c5ccc(-c6cccc7c6ccc6ccccc67)cc5)cc4)cc3)cccc2c1 ; c1cc(-c2cccc3ccccc23)cc(N(c2ccc(-c3ccc4sc5ccccc5c4c3)cc2)c2ccc(-c3cccc4c3ccc3ccccc34)cc2)c1]

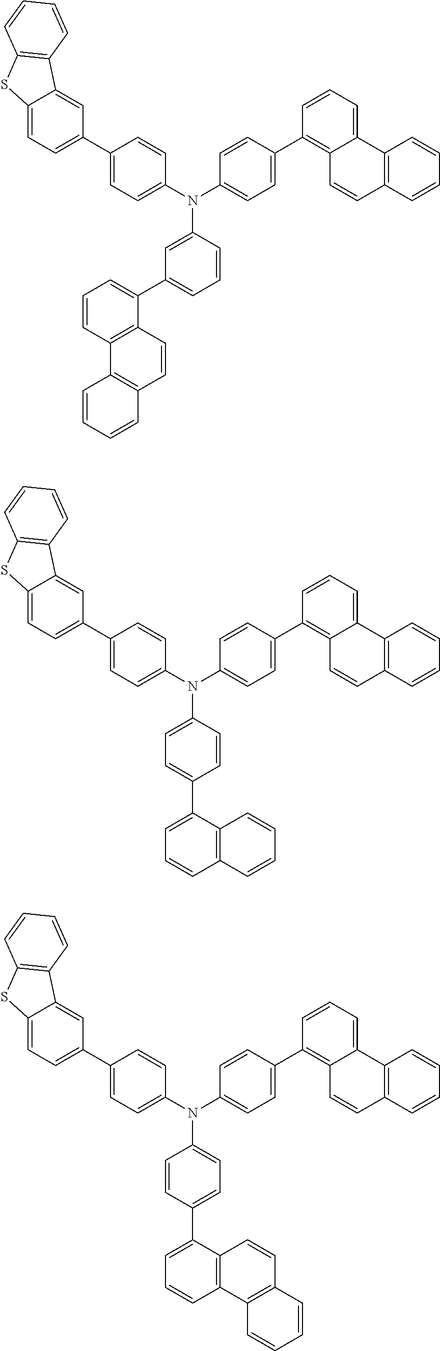 [SMILES: c1cc(-c2cccc3c2ccc2ccccc23)cc(N(c2ccc(-c3ccc4sc5ccccc5c4c3)cc2)c2ccc(-c3cccc4c3ccc3ccccc34)cc2)c1 ; c1ccc2c(-c3ccc(N(c4ccc(-c5ccc6sc7ccccc7c6c5)cc4)c4ccc(-c5cccc6c5ccc5ccccc56)cc4)cc3)cccc2c1 ; c1ccc2c(c1)ccc1c(-c3ccc(N(c4ccc(-c5ccc6sc7ccccc7c6c5)cc4)c4ccc(-c5cccc6c5ccc5ccccc56)cc4)cc3)cccc12]

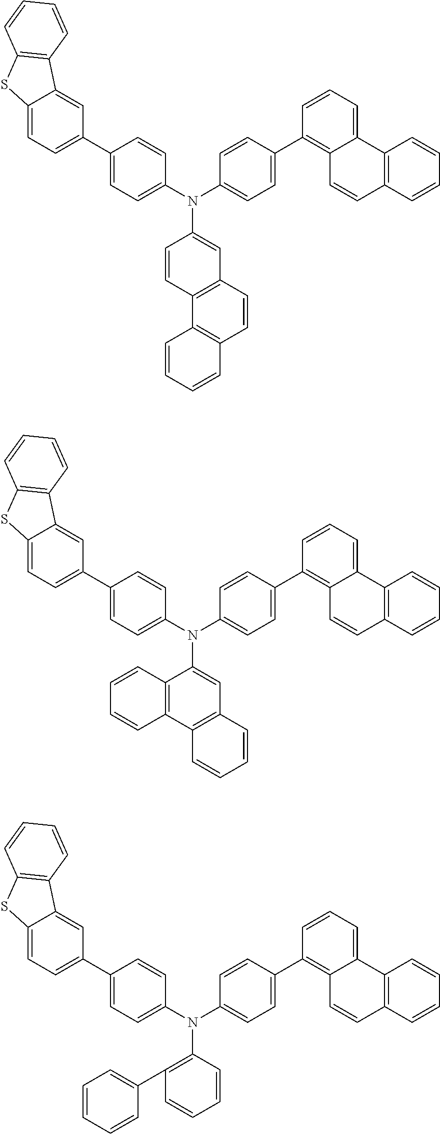 [SMILES: c1ccc2c(c1)ccc1cc(N(c3ccc(-c4ccc5sc6ccccc6c5c4)cc3)c3ccc(-c4cccc5c4ccc4ccccc45)cc3)ccc12 ; c1ccc2c(c1)ccc1c(-c3ccc(N(c4ccc(-c5ccc6sc7ccccc7c6c5)cc4)c4cc5ccccc5c5ccccc45)cc3)cccc12 ; c1ccc(-c2ccccc2N(c2ccc(-c3ccc4sc5ccccc5c4c3)cc2)c2ccc(-c3cccc4c3ccc3ccccc34)cc2)cc1]

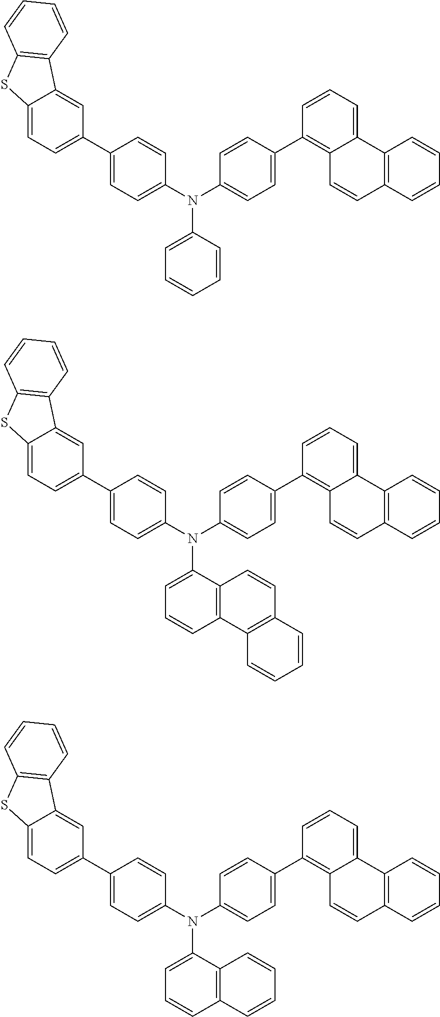 [SMILES: c1ccc(N(c2ccc(-c3ccc4sc5ccccc5c4c3)cc2)c2ccc(-c3cccc4c3ccc3ccccc34)cc2)cc1 ; c1ccc2c(c1)ccc1c(-c3ccc(N(c4ccc(-c5ccc6sc7ccccc7c6c5)cc4)c4cccc5c4ccc4ccccc45)cc3)cccc12 ; c1ccc2c(N(c3ccc(-c4ccc5sc6ccccc6c5c4)cc3)c3ccc(-c4cccc5c4ccc4ccccc45)cc3)cccc2c1]

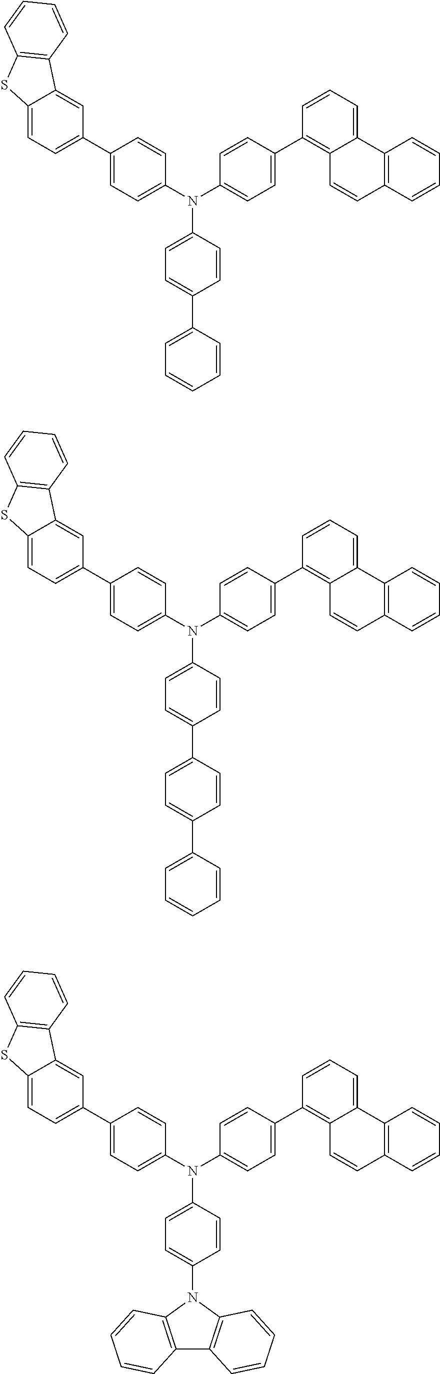 [SMILES: c1ccc(-c2ccc(N(c3ccc(-c4ccc5sc6ccccc6c5c4)cc3)c3ccc(-c4cccc5c4ccc4ccccc45)cc3)cc2)cc1 ; c1ccc(-c2ccc(-c3ccc(N(c4ccc(-c5ccc6sc7ccccc7c6c5)cc4)c4ccc(-c5cccc6c5ccc5ccccc56)cc4)cc3)cc2)cc1 ; c1ccc2c(c1)ccc1c(-c3ccc(N(c4ccc(-c5ccc6sc7ccccc7c6c5)cc4)c4ccc(-n5c6ccccc6c6ccccc65)cc4)cc3)cccc12]

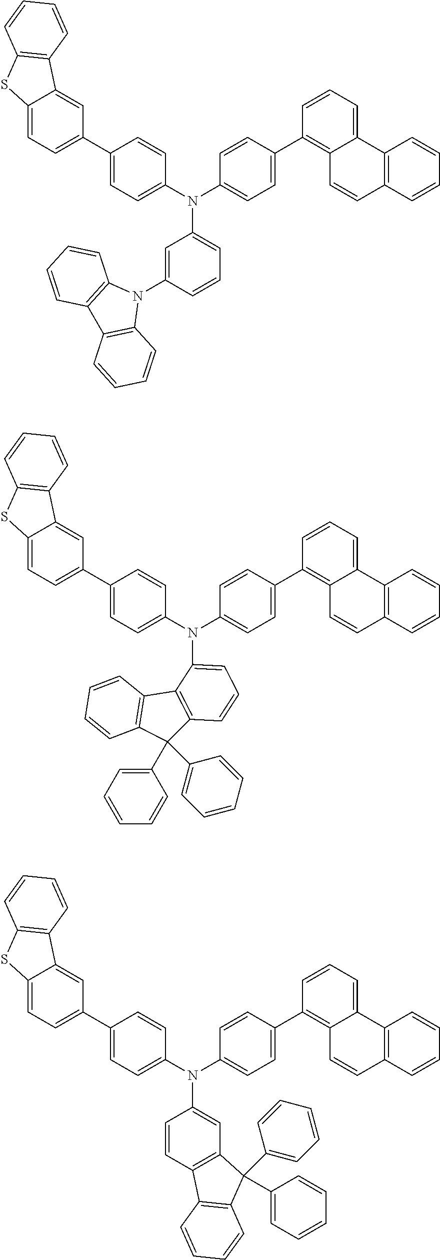 [SMILES: c1cc(N(c2ccc(-c3ccc4sc5ccccc5c4c3)cc2)c2ccc(-c3cccc4c3ccc3ccccc34)cc2)cc(-n2c3ccccc3c3ccccc32)c1 ; c1ccc(C2(c3ccccc3)c3ccccc3-c3c(N(c4ccc(-c5ccc6sc7ccccc7c6c5)cc4)c4ccc(-c5cccc6c5ccc5ccccc56)cc4)cccc32)cc1 ; c1ccc(C2(c3ccccc3)c3ccccc3-c3ccc(N(c4ccc(-c5ccc6sc7ccccc7c6c5)cc4)c4ccc(-c5cccc6c5ccc5ccccc56)cc4)cc32)cc1]

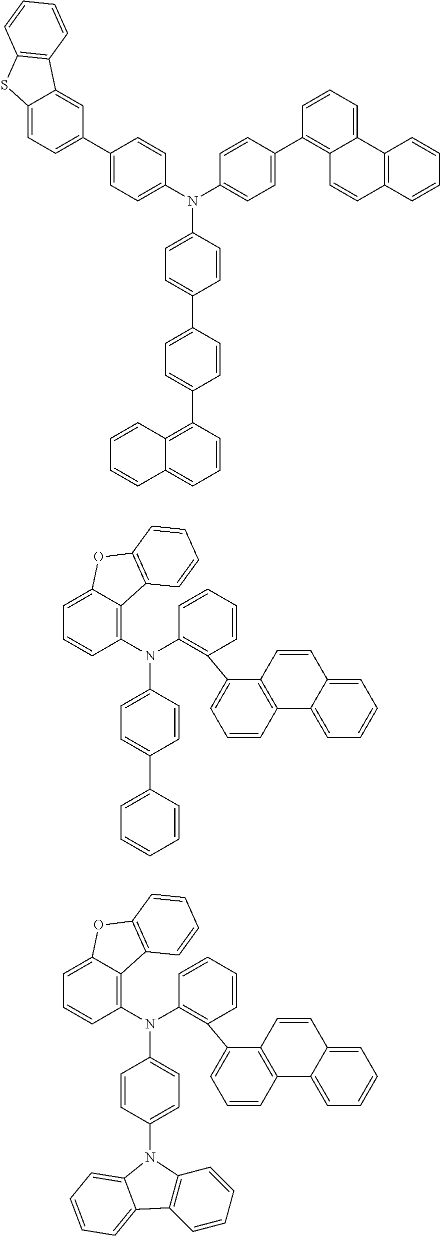

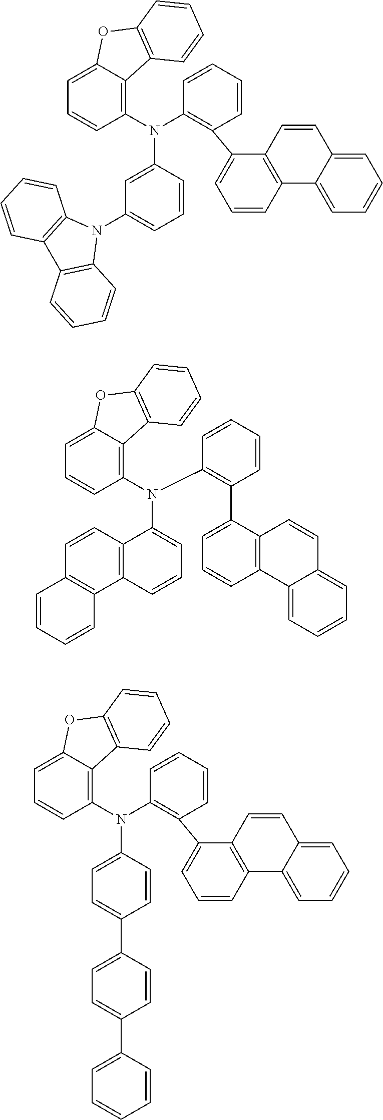 [SMILES: c1cc(N(c2ccccc2-c2cccc3c2ccc2ccccc23)c2cccc3oc4ccccc4c23)cc(-n2c3ccccc3c3ccccc32)c1 ; c1ccc(N(c2cccc3c2ccc2ccccc23)c2cccc3oc4ccccc4c23)c(-c2cccc3c2ccc2ccccc23)c1 ; c1ccc(-c2ccc(-c3ccc(N(c4ccccc4-c4cccc5c4ccc4ccccc45)c4cccc5oc6ccccc6c45)cc3)cc2)cc1]

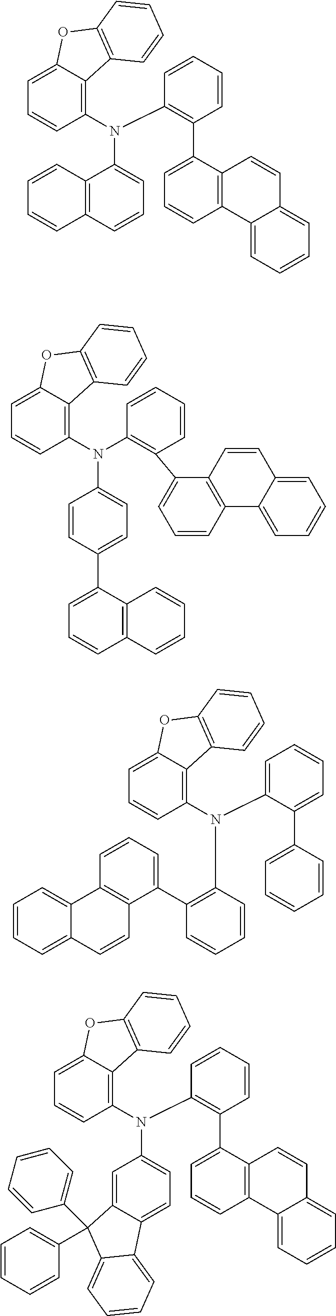 [SMILES: c1ccc(N(c2cccc3ccccc23)c2cccc3oc4ccccc4c23)c(-c2cccc3c2ccc2ccccc23)c1 ; c1ccc(N(c2ccc(-c3cccc4ccccc34)cc2)c2cccc3oc4ccccc4c23)c(-c2cccc3c2ccc2ccccc23)c1 ; c1ccc(-c2ccccc2N(c2ccccc2-c2cccc3c2ccc2ccccc23)c2cccc3oc4ccccc4c23)cc1 ; c1ccc(C2(c3ccccc3)c3ccccc3-c3ccc(N(c4ccccc4-c4cccc5c4ccc4ccccc45)c4cccc5oc6ccccc6c45)cc32)cc1]

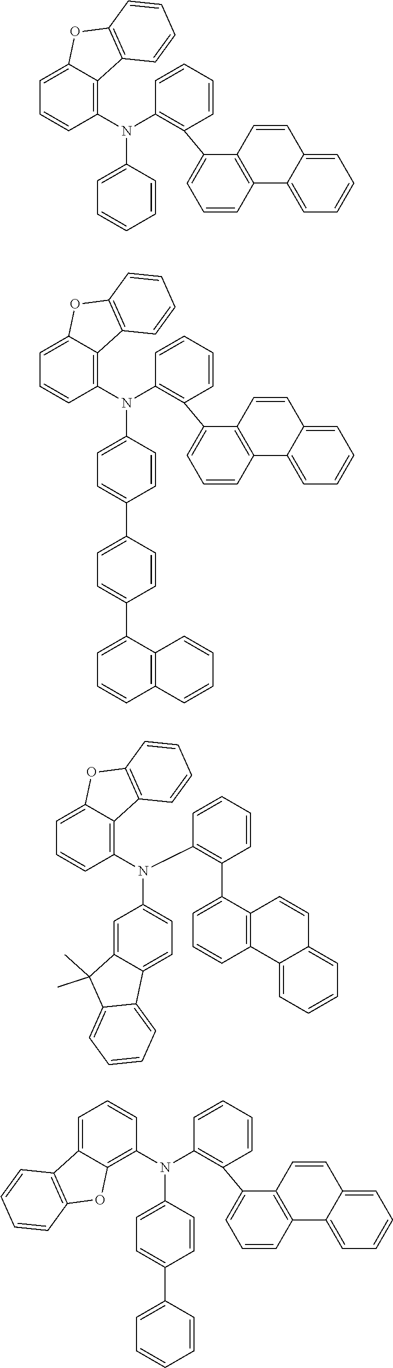

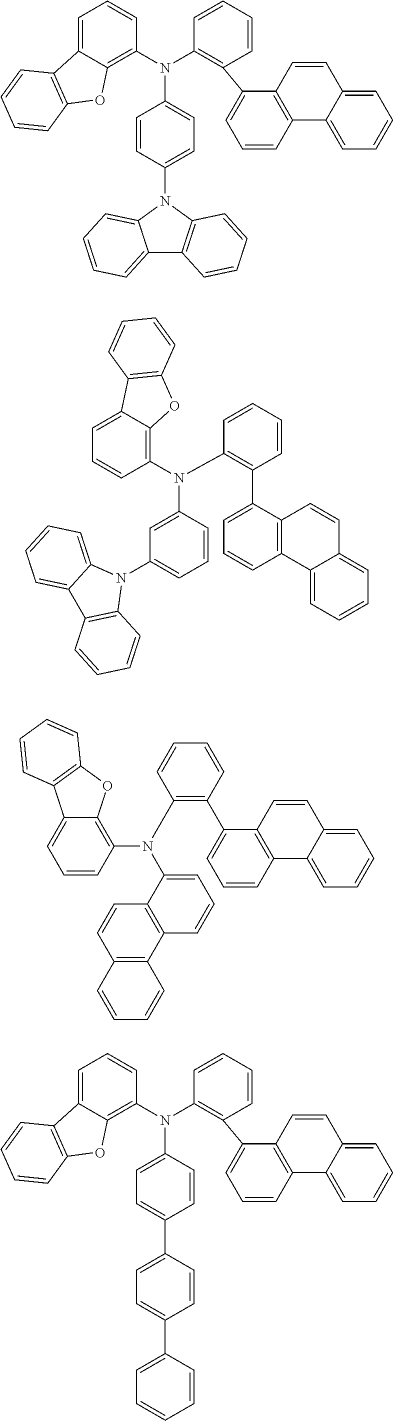

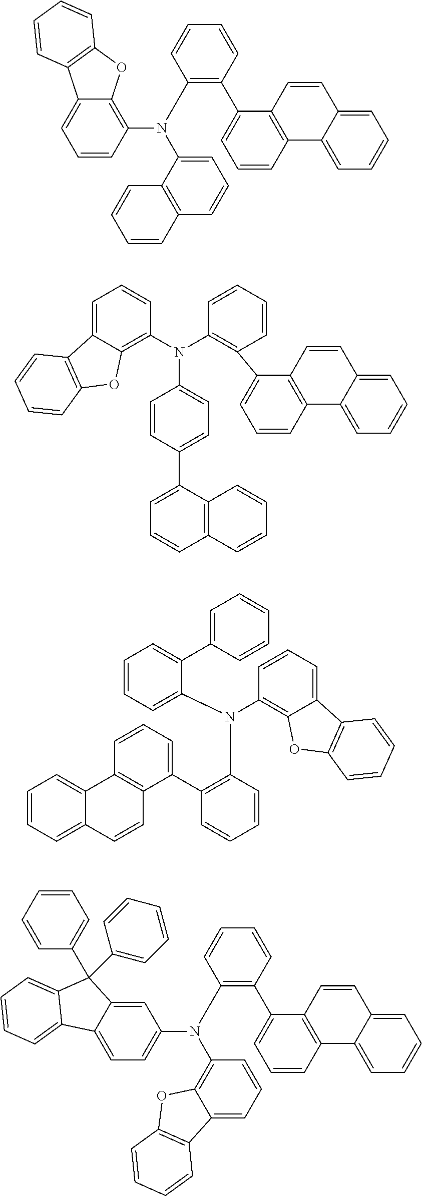

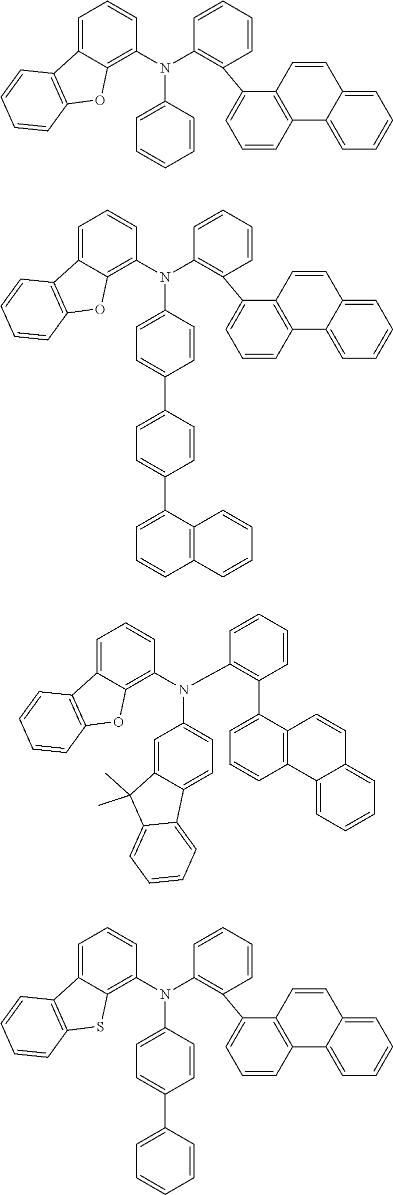 [SMILES: c1ccc(N(c2ccccc2-c2cccc3c2ccc2ccccc23)c2cccc3c2oc2ccccc23)cc1 ; c1ccc(N(c2ccc(-c3ccc(-c4cccc5ccccc45)cc3)cc2)c2cccc3c2oc2ccccc23)c(-c2cccc3c2ccc2ccccc23)c1 ; CC1(C)c2ccccc2-c2ccc(N(c3ccccc3-c3cccc4c3ccc3ccccc34)c3cccc4c3oc3ccccc34)cc21 ; c1ccc(-c2ccc(N(c3ccccc3-c3cccc4c3ccc3ccccc34)c3cccc4c3sc3ccccc34)cc2)cc1]

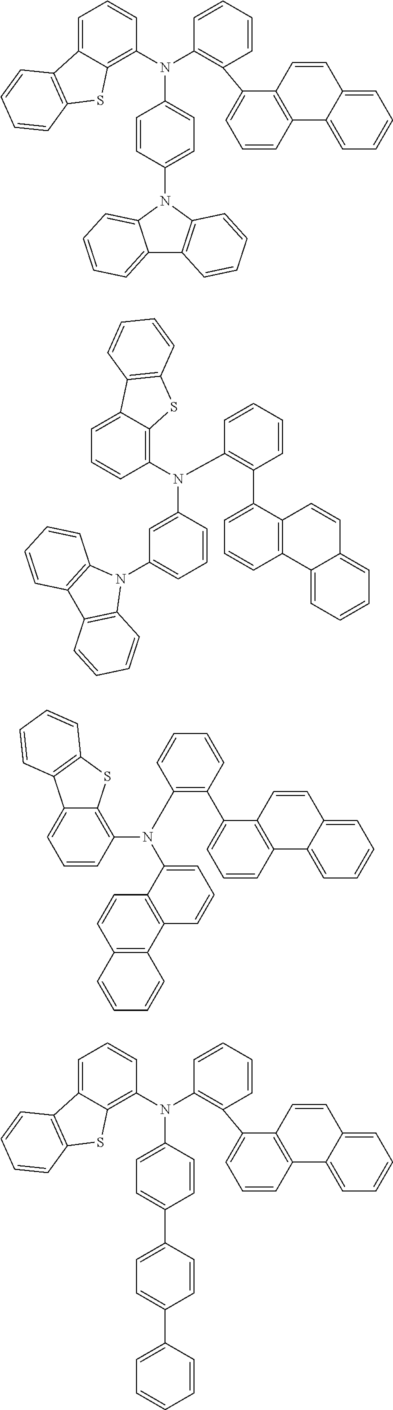

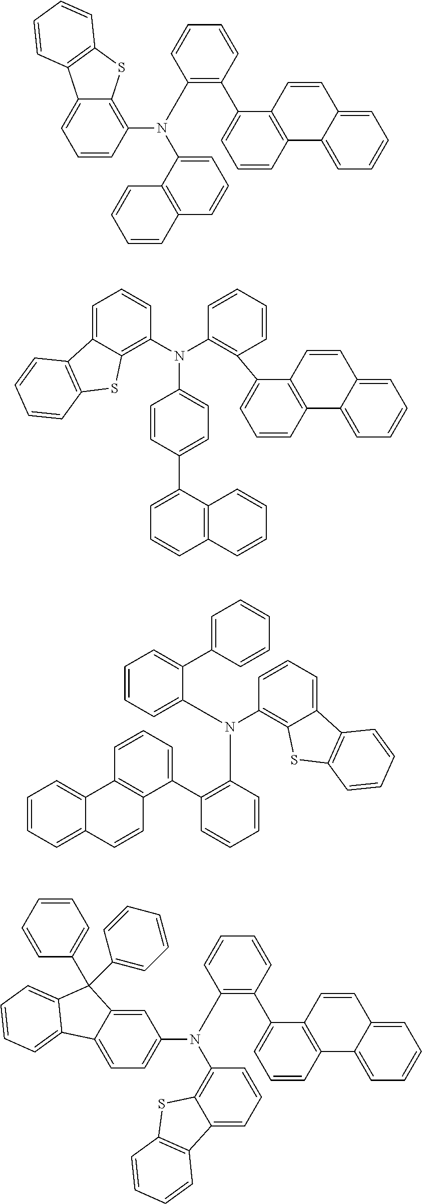

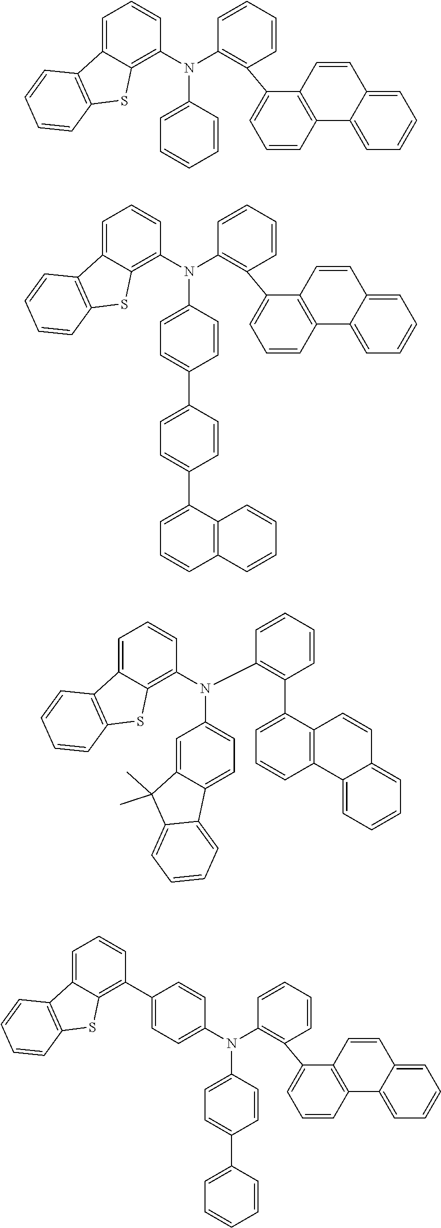

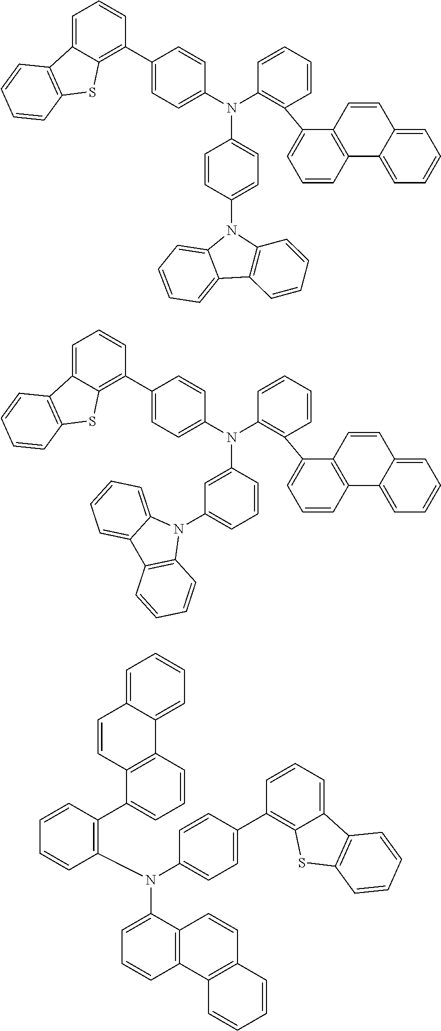

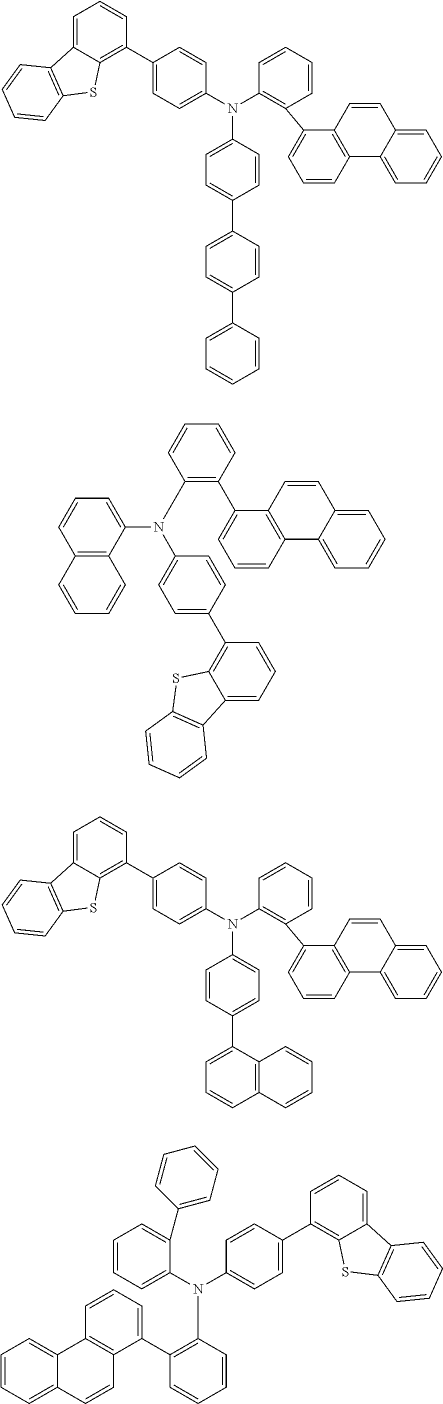

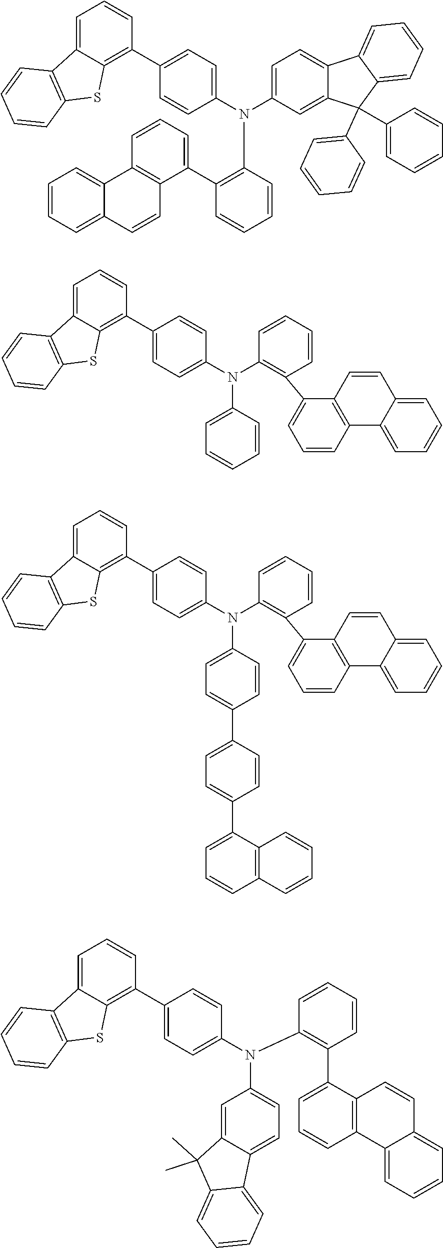

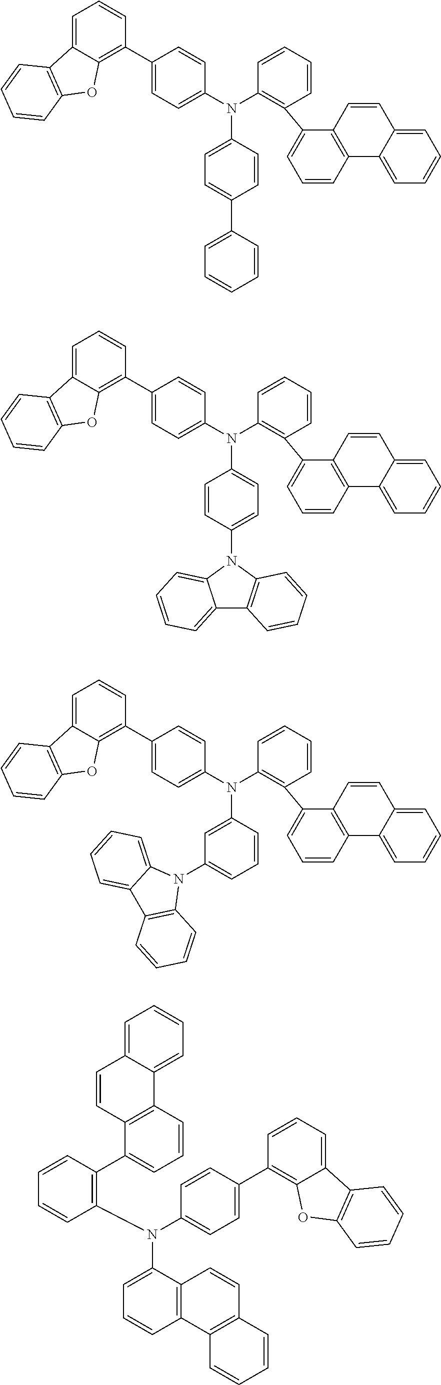

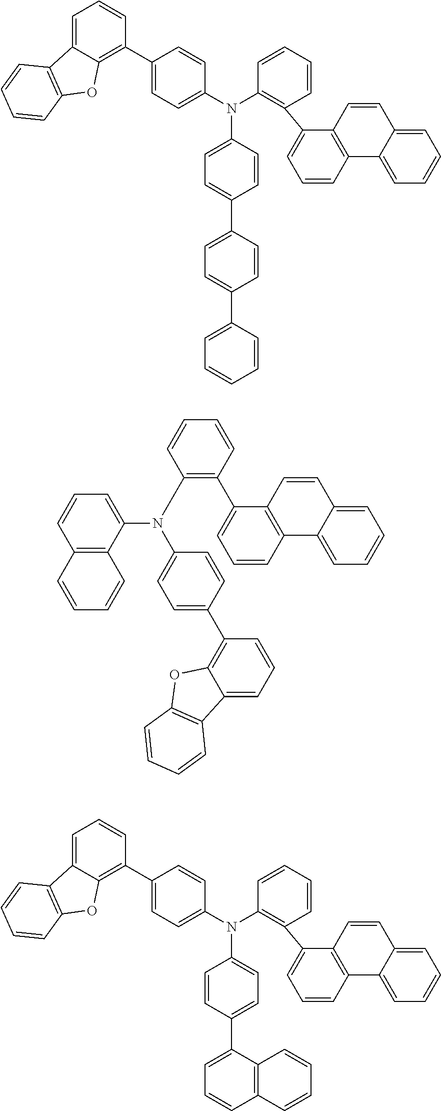

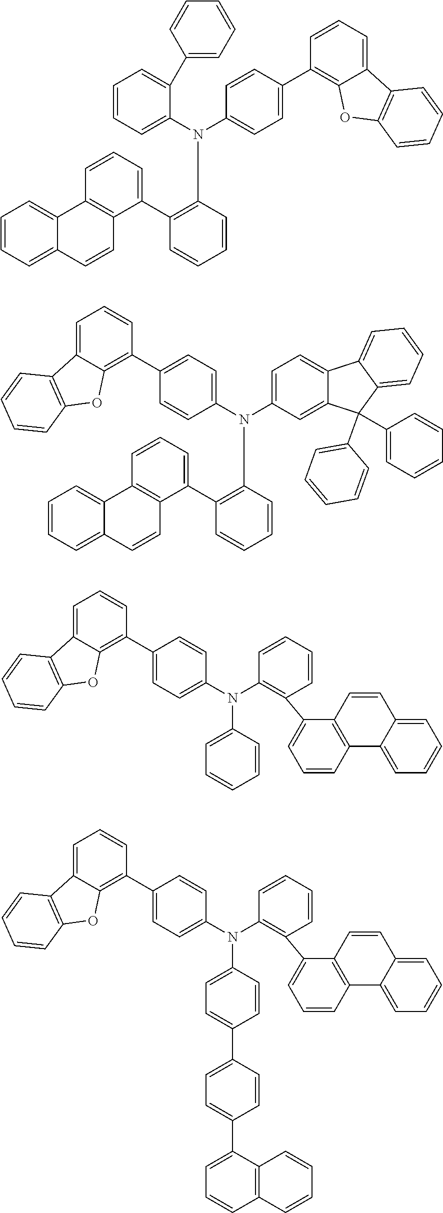

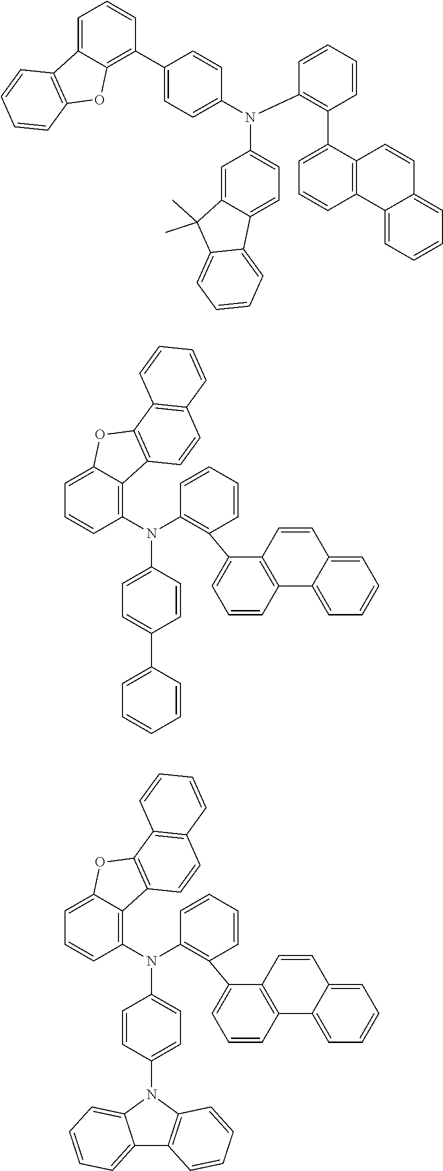

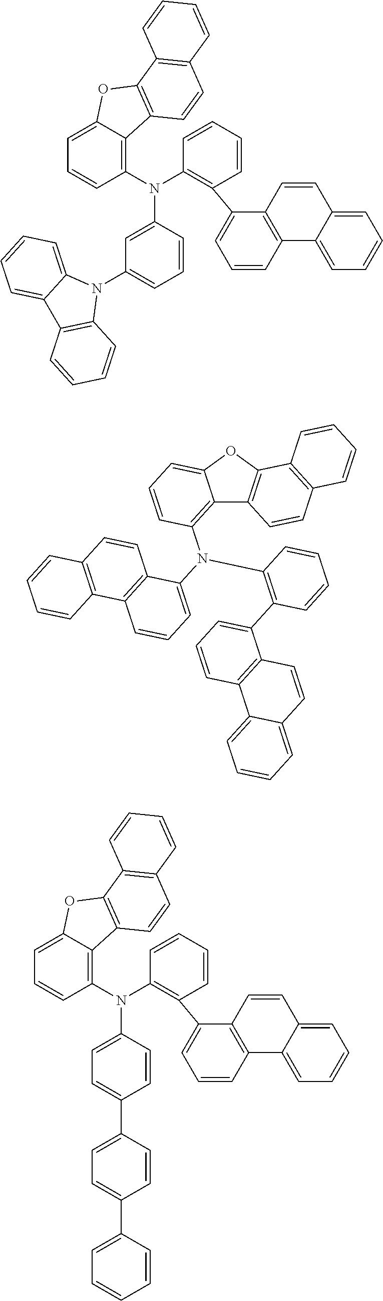 [SMILES: c1cc(N(c2ccccc2-c2cccc3c2ccc2ccccc23)c2cccc3oc4c5ccccc5ccc4c23)cc(-n2c3ccccc3c3ccccc32)c1 ; c1ccc(N(c2cccc3c2ccc2ccccc23)c2cccc3oc4c5ccccc5ccc4c23)c(-c2cccc3c2ccc2ccccc23)c1 ; c1ccc(-c2ccc(-c3ccc(N(c4ccccc4-c4cccc5c4ccc4ccccc45)c4cccc5oc6c7ccccc7ccc6c45)cc3)cc2)cc1]

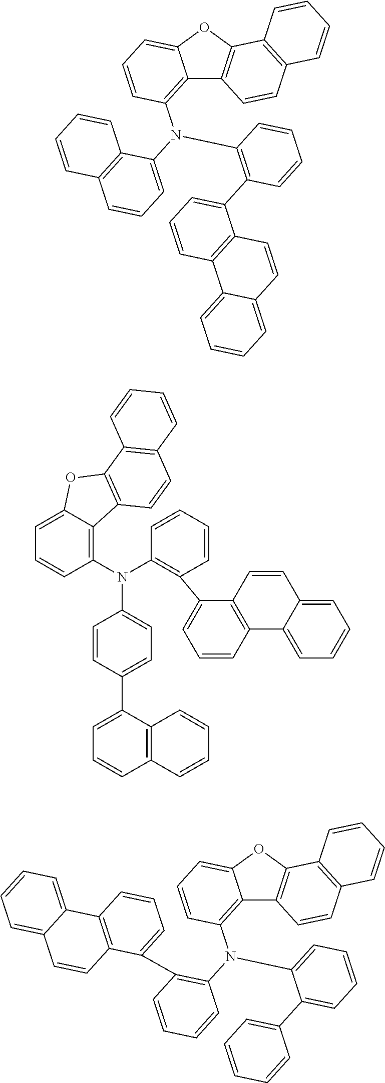 [SMILES: c1ccc(N(c2cccc3ccccc23)c2cccc3oc4c5ccccc5ccc4c23)c(-c2cccc3c2ccc2ccccc23)c1 ; c1ccc(N(c2ccc(-c3cccc4ccccc34)cc2)c2cccc3oc4c5ccccc5ccc4c23)c(-c2cccc3c2ccc2ccccc23)c1 ; c1ccc(-c2ccccc2N(c2ccccc2-c2cccc3c2ccc2ccccc23)c2cccc3oc4c5ccccc5ccc4c23)cc1]

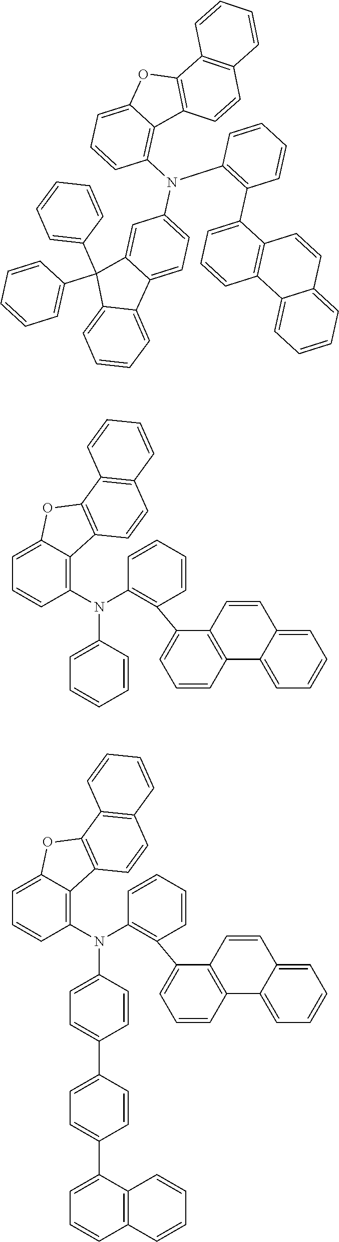 [SMILES: c1ccc(C2(c3ccccc3)c3ccccc3-c3ccc(N(c4ccccc4-c4cccc5c4ccc4ccccc45)c4cccc5oc6c7ccccc7ccc6c45)cc32)cc1 ; c1ccc(N(c2ccccc2-c2cccc3c2ccc2ccccc23)c2cccc3oc4c5ccccc5ccc4c23)cc1 ; c1ccc(N(c2ccc(-c3ccc(-c4cccc5ccccc45)cc3)cc2)c2cccc3oc4c5ccccc5ccc4c23)c(-c2cccc3c2ccc2ccccc23)c1]

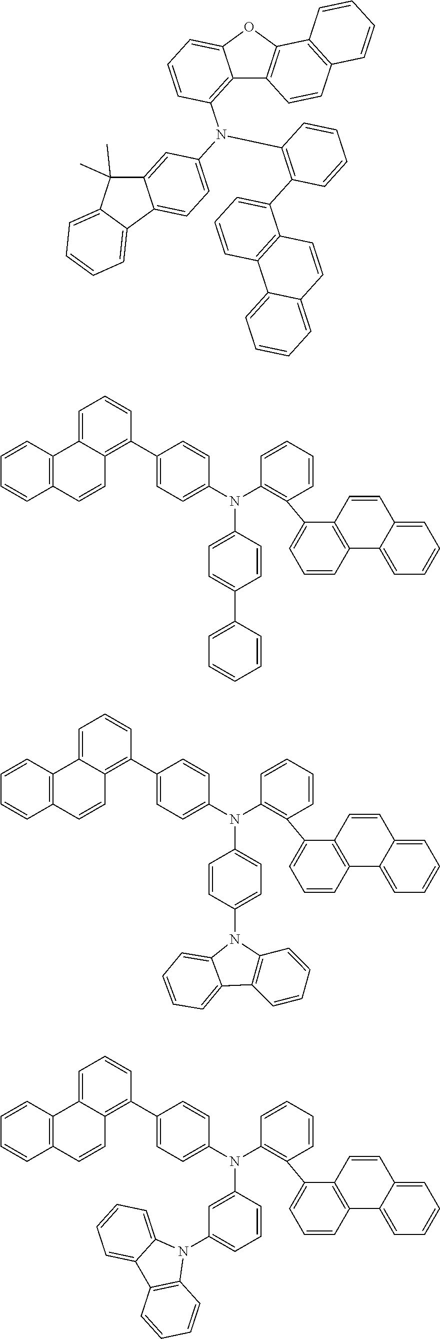 [SMILES: CC1(C)c2ccccc2-c2ccc(N(c3ccccc3-c3cccc4c3ccc3ccccc34)c3cccc4oc5c6ccccc6ccc5c34)cc21 ; c1ccc(-c2ccc(N(c3ccc(-c4cccc5c4ccc4ccccc45)cc3)c3ccccc3-c3cccc4c3ccc3ccccc34)cc2)cc1 ; c1ccc(N(c2ccc(-c3cccc4c3ccc3ccccc34)cc2)c2ccc(-n3c4ccccc4c4ccccc43)cc2)c(-c2cccc3c2ccc2ccccc23)c1 ; c1cc(N(c2ccc(-c3cccc4c3ccc3ccccc34)cc2)c2ccccc2-c2cccc3c2ccc2ccccc23)cc(-n2c3ccccc3c3ccccc32)c1]

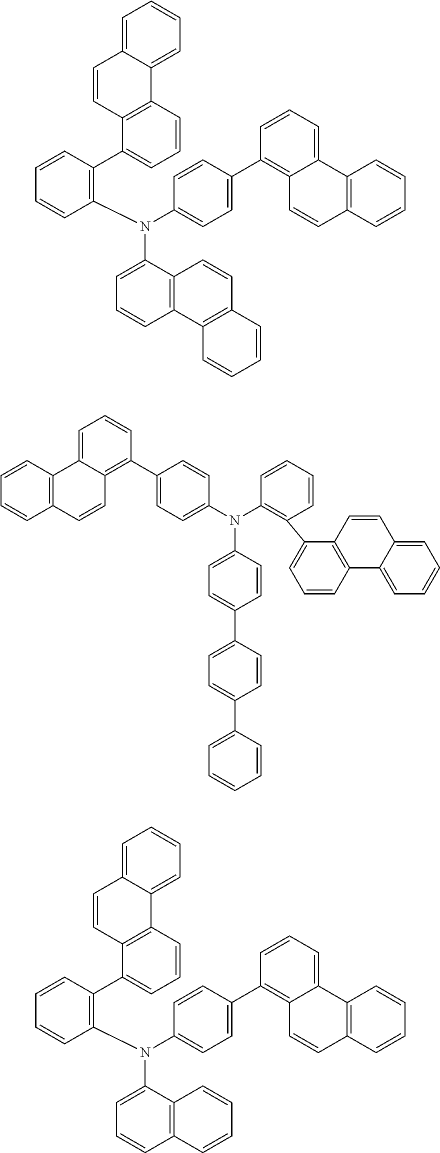 [SMILES: c1ccc(N(c2ccc(-c3cccc4c3ccc3ccccc34)cc2)c2cccc3c2ccc2ccccc23)c(-c2cccc3c2ccc2ccccc23)c1 ; c1ccc(-c2ccc(-c3ccc(N(c4ccc(-c5cccc6c5ccc5ccccc56)cc4)c4ccccc4-c4cccc5c4ccc4ccccc45)cc3)cc2)cc1 ; c1ccc(N(c2ccc(-c3cccc4c3ccc3ccccc34)cc2)c2cccc3ccccc23)c(-c2cccc3c2ccc2ccccc23)c1]

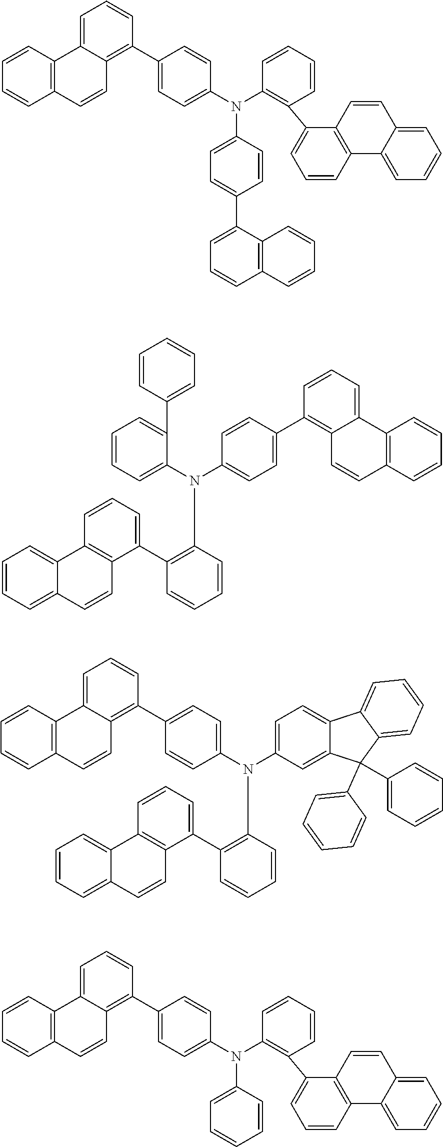 [SMILES: c1ccc(N(c2ccc(-c3cccc4ccccc34)cc2)c2ccc(-c3cccc4c3ccc3ccccc34)cc2)c(-c2cccc3c2ccc2ccccc23)c1 ; c1ccc(-c2ccccc2N(c2ccc(-c3cccc4c3ccc3ccccc34)cc2)c2ccccc2-c2cccc3c2ccc2ccccc23)cc1 ; c1ccc(C2(c3ccccc3)c3ccccc3-c3ccc(N(c4ccc(-c5cccc6c5ccc5ccccc56)cc4)c4ccccc4-c4cccc5c4ccc4ccccc45)cc32)cc1 ; c1ccc(N(c2ccc(-c3cccc4c3ccc3ccccc34)cc2)c2ccccc2-c2cccc3c2ccc2ccccc23)cc1]

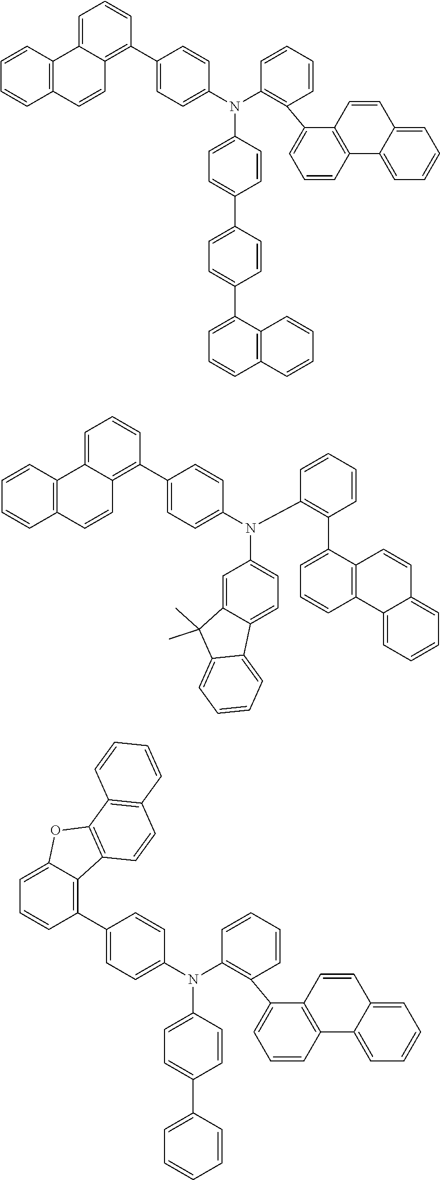 [SMILES: c1ccc(N(c2ccc(-c3ccc(-c4cccc5ccccc45)cc3)cc2)c2ccc(-c3cccc4c3ccc3ccccc34)cc2)c(-c2cccc3c2ccc2ccccc23)c1 ; CC1(C)c2ccccc2-c2ccc(N(c3ccc(-c4cccc5c4ccc4ccccc45)cc3)c3ccccc3-c3cccc4c3ccc3ccccc34)cc21 ; c1ccc(-c2ccc(N(c3ccc(-c4cccc5oc6c7ccccc7ccc6c45)cc3)c3ccccc3-c3cccc4c3ccc3ccccc34)cc2)cc1]

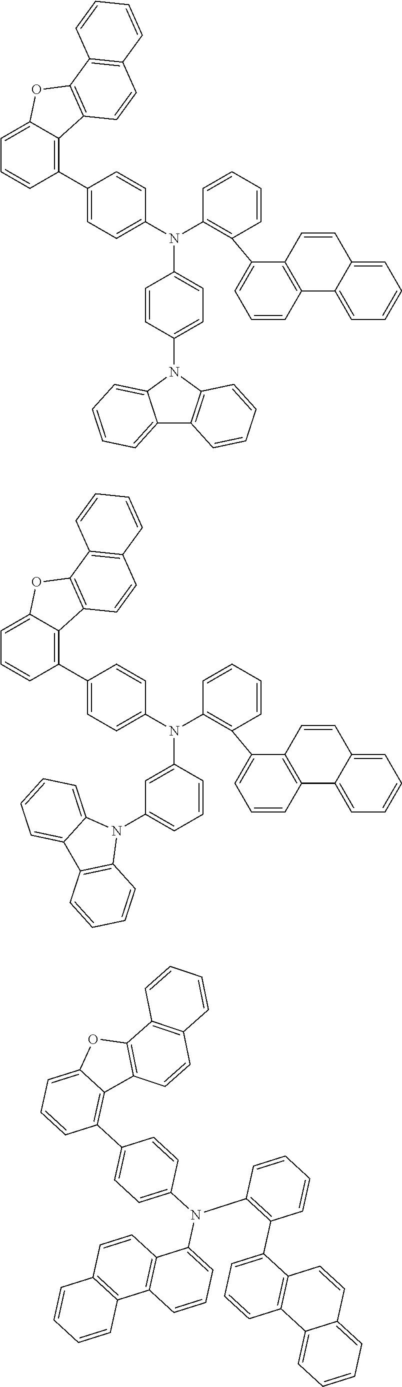 [SMILES: c1ccc(N(c2ccc(-c3cccc4oc5c6ccccc6ccc5c34)cc2)c2ccc(-n3c4ccccc4c4ccccc43)cc2)c(-c2cccc3c2ccc2ccccc23)c1 ; c1cc(N(c2ccc(-c3cccc4oc5c6ccccc6ccc5c34)cc2)c2ccccc2-c2cccc3c2ccc2ccccc23)cc(-n2c3ccccc3c3ccccc32)c1 ; c1ccc(N(c2ccc(-c3cccc4oc5c6ccccc6ccc5c34)cc2)c2cccc3c2ccc2ccccc23)c(-c2cccc3c2ccc2ccccc23)c1]

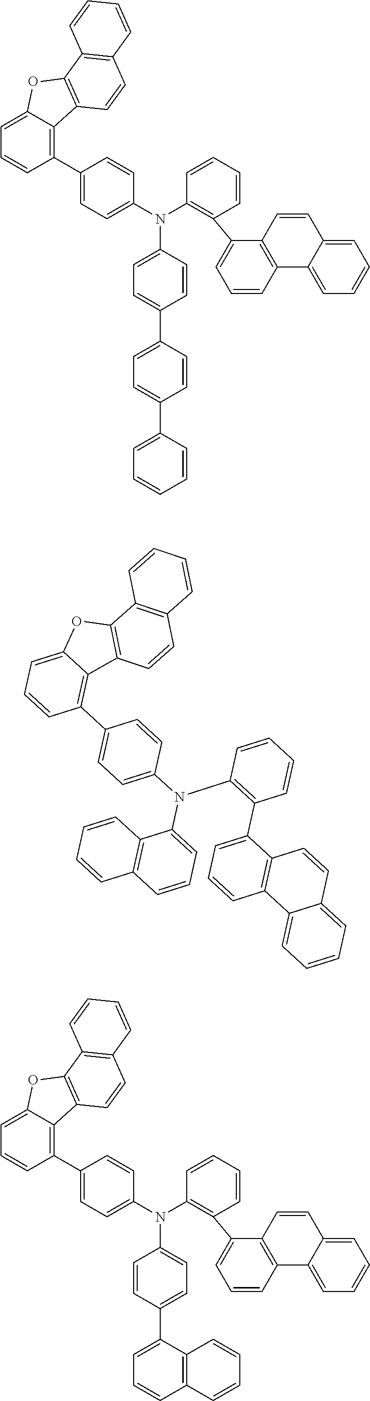 [SMILES: c1ccc(-c2ccc(-c3ccc(N(c4ccc(-c5cccc6oc7c8ccccc8ccc7c56)cc4)c4ccccc4-c4cccc5c4ccc4ccccc45)cc3)cc2)cc1 ; c1ccc(N(c2ccc(-c3cccc4oc5c6ccccc6ccc5c34)cc2)c2cccc3ccccc23)c(-c2cccc3c2ccc2ccccc23)c1 ; c1ccc(N(c2ccc(-c3cccc4ccccc34)cc2)c2ccc(-c3cccc4oc5c6ccccc6ccc5c34)cc2)c(-c2cccc3c2ccc2ccccc23)c1]

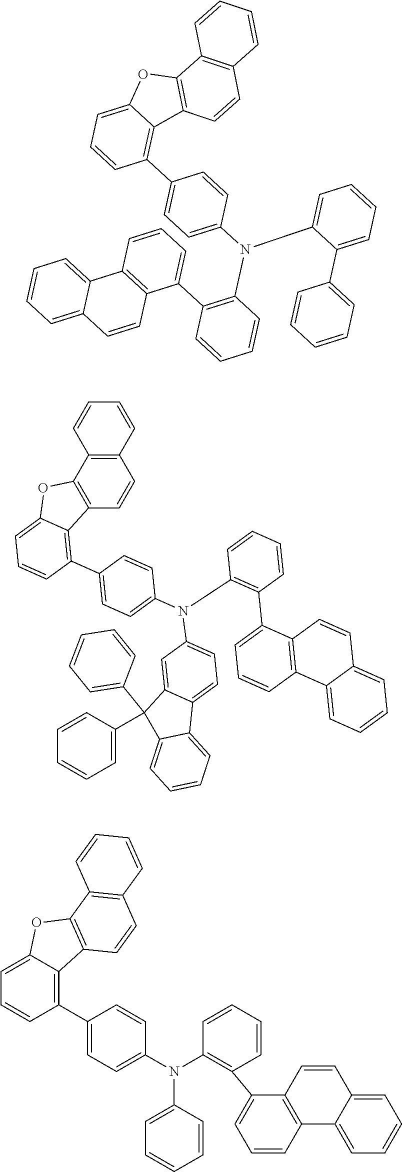 [SMILES: c1ccc(-c2ccccc2N(c2ccc(-c3cccc4oc5c6ccccc6ccc5c34)cc2)c2ccccc2-c2cccc3c2ccc2ccccc23)cc1 ; c1ccc(C2(c3ccccc3)c3ccccc3-c3ccc(N(c4ccc(-c5cccc6oc7c8ccccc8ccc7c56)cc4)c4ccccc4-c4cccc5c4ccc4ccccc45)cc32)cc1 ; c1ccc(N(c2ccc(-c3cccc4oc5c6ccccc6ccc5c34)cc2)c2ccccc2-c2cccc3c2ccc2ccccc23)cc1]

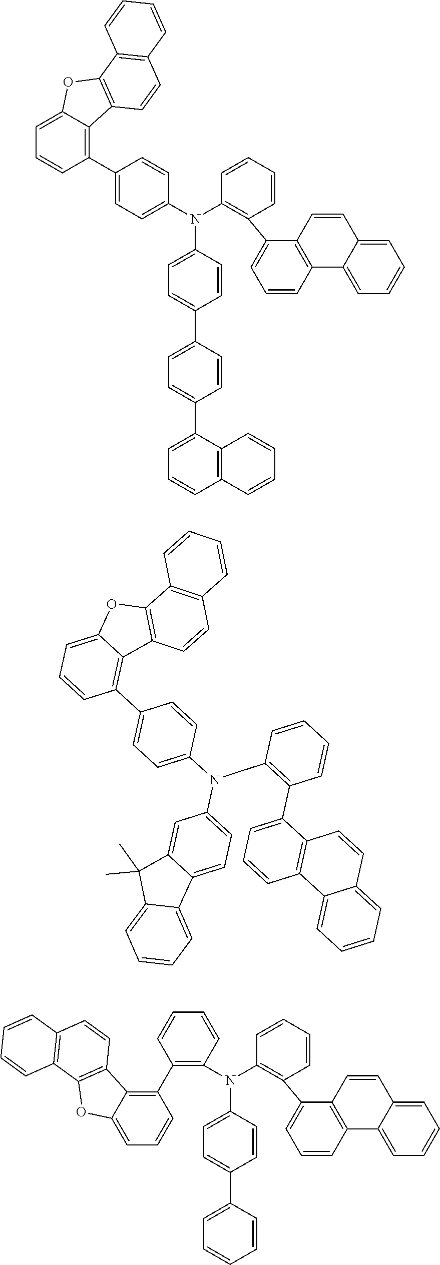

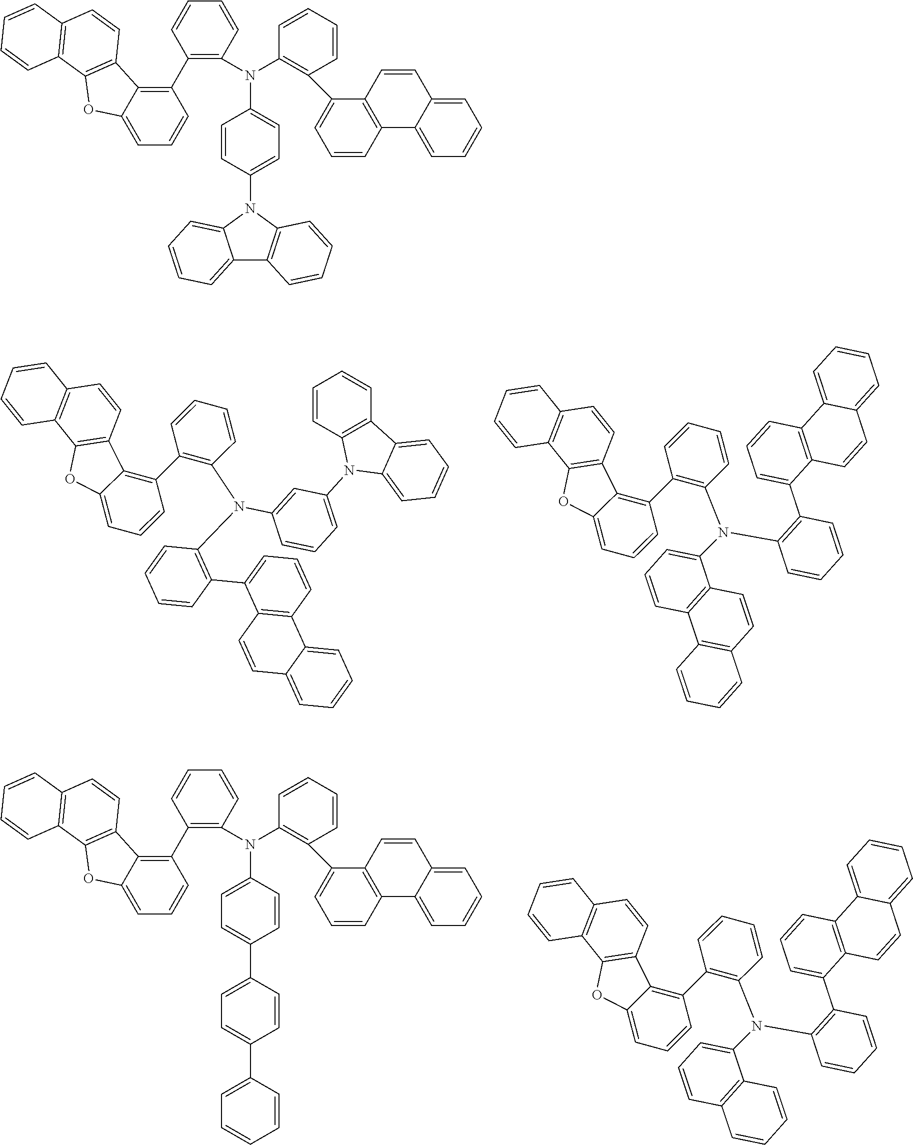 [SMILES: c1ccc(N(c2ccc(-n3c4ccccc4c4ccccc43)cc2)c2ccccc2-c2cccc3oc4c5ccccc5ccc4c23)c(-c2cccc3c2ccc2ccccc23)c1 ; c1cc(N(c2ccccc2-c2cccc3c2ccc2ccccc23)c2ccccc2-c2cccc3oc4c5ccccc5ccc4c23)cc(-n2c3ccccc3c3ccccc32)c1 ; c1ccc(N(c2ccccc2-c2cccc3oc4c5ccccc5ccc4c23)c2cccc3c2ccc2ccccc23)c(-c2cccc3c2ccc2ccccc23)c1 ; c1ccc(-c2ccc(-c3ccc(N(c4ccccc4-c4cccc5c4ccc4ccccc45)c4ccccc4-c4cccc5oc6c7ccccc7ccc6c45)cc3)cc2)cc1 ; c1ccc(N(c2ccccc2-c2cccc3oc4c5ccccc5ccc4c23)c2cccc3ccccc23)c(-c2cccc3c2ccc2ccccc23)c1]

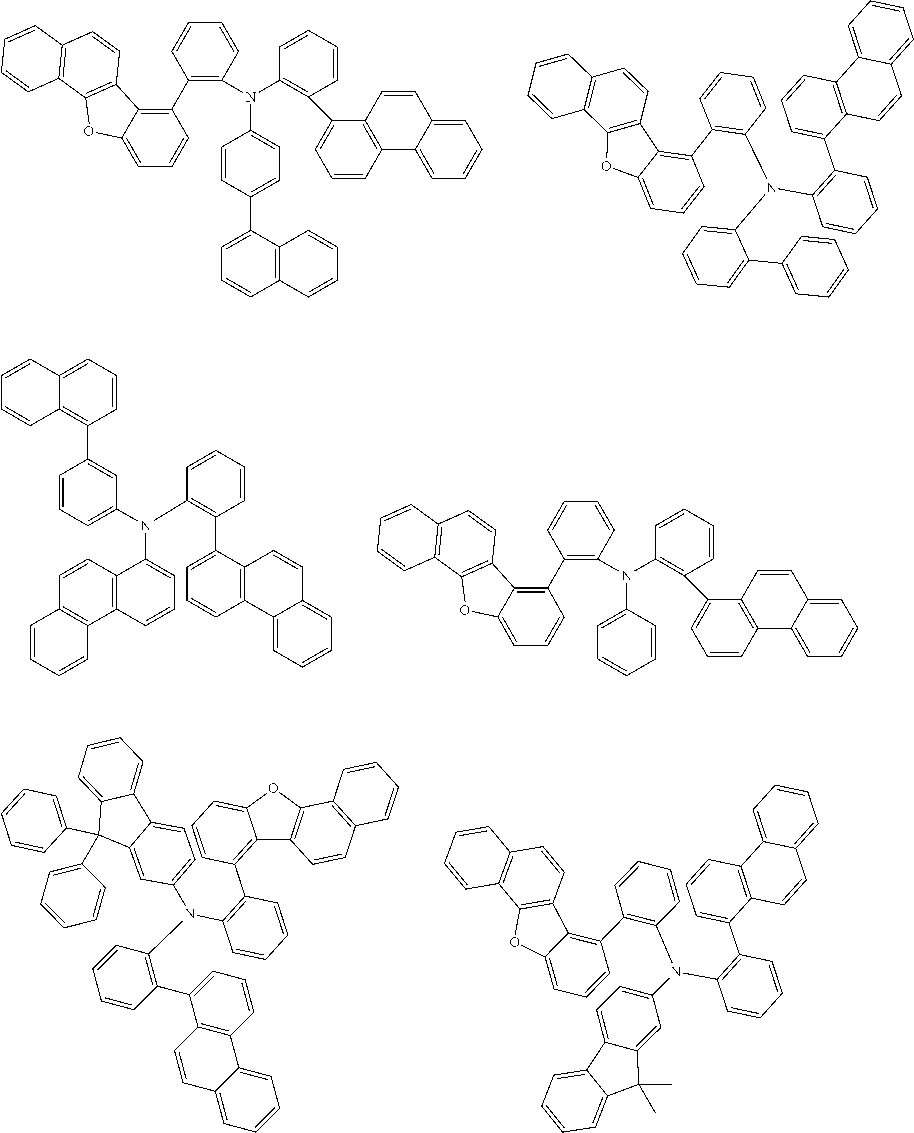

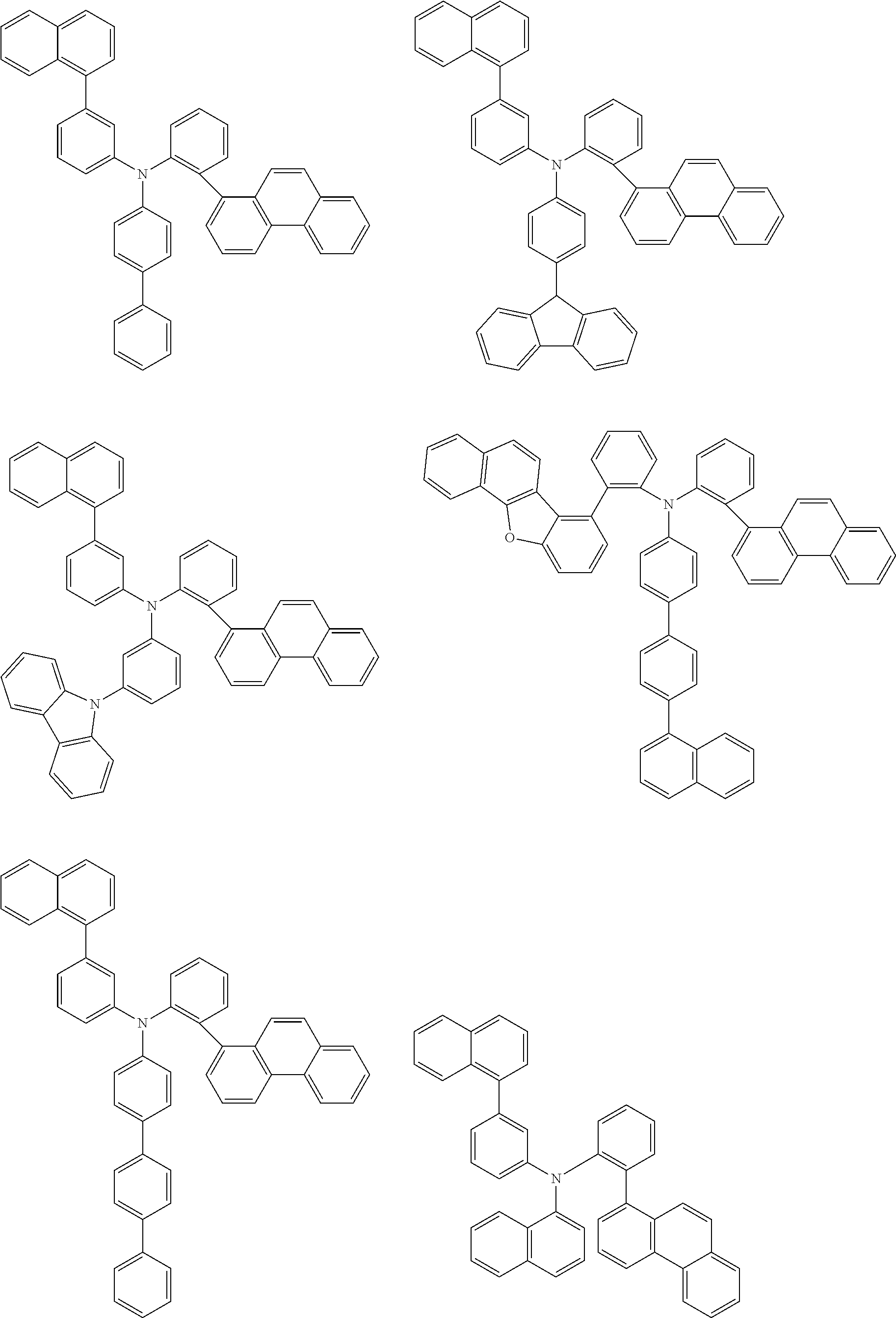

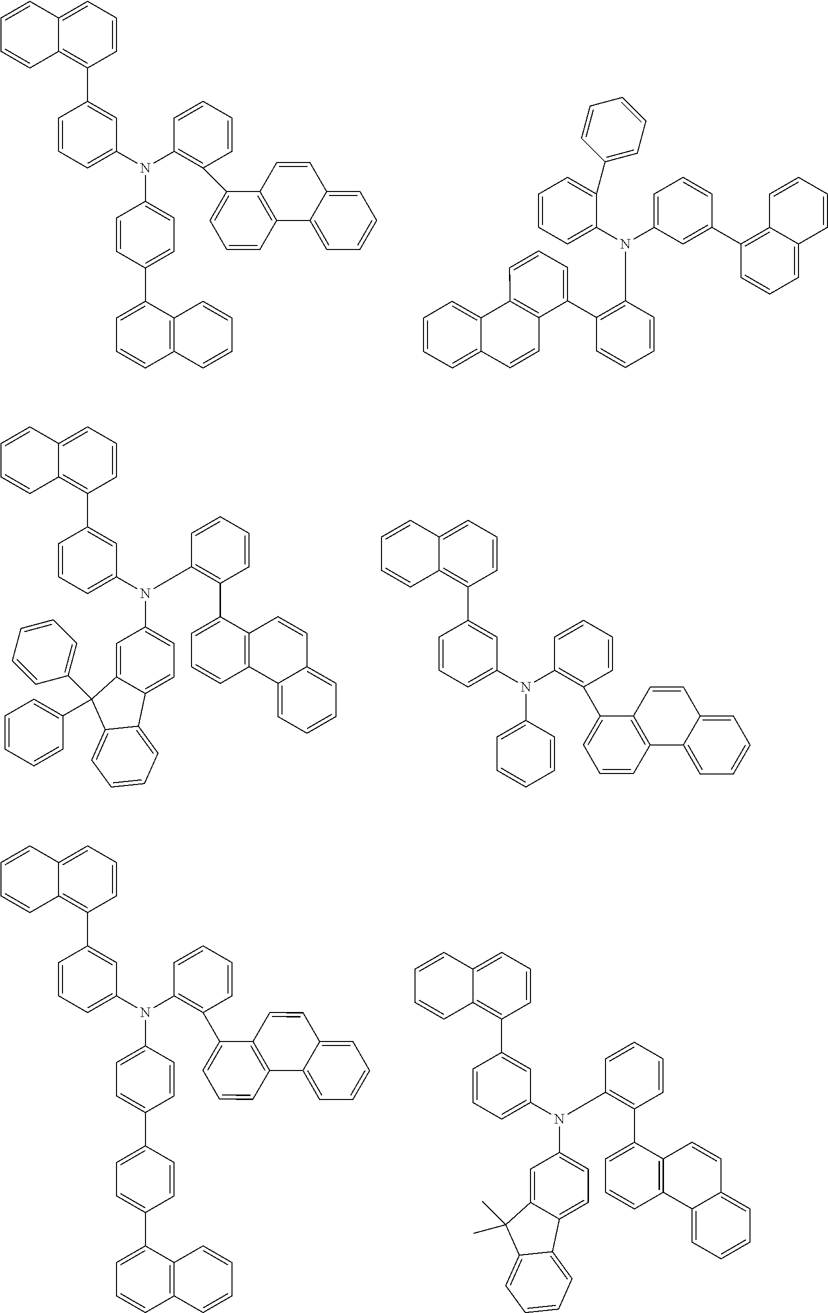

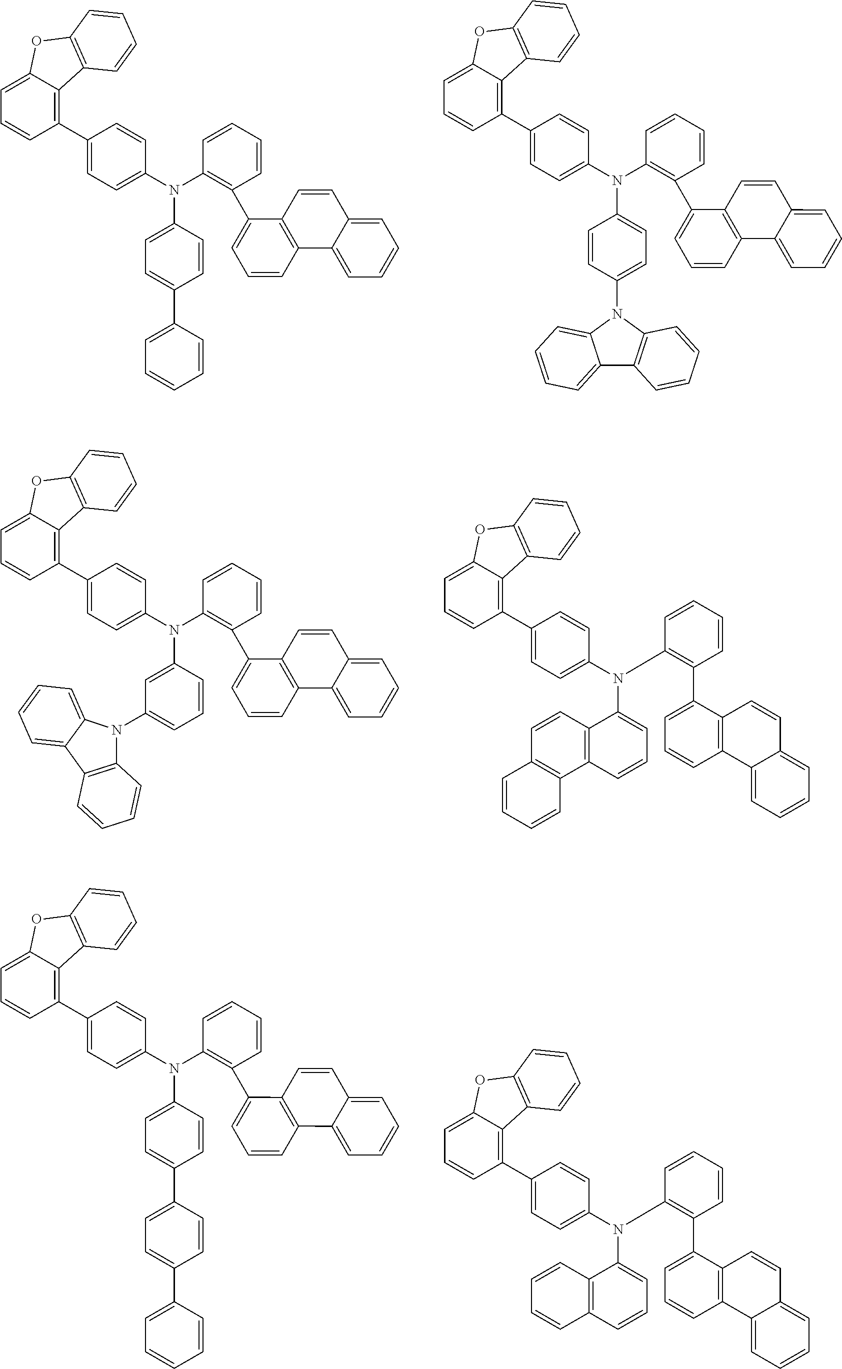

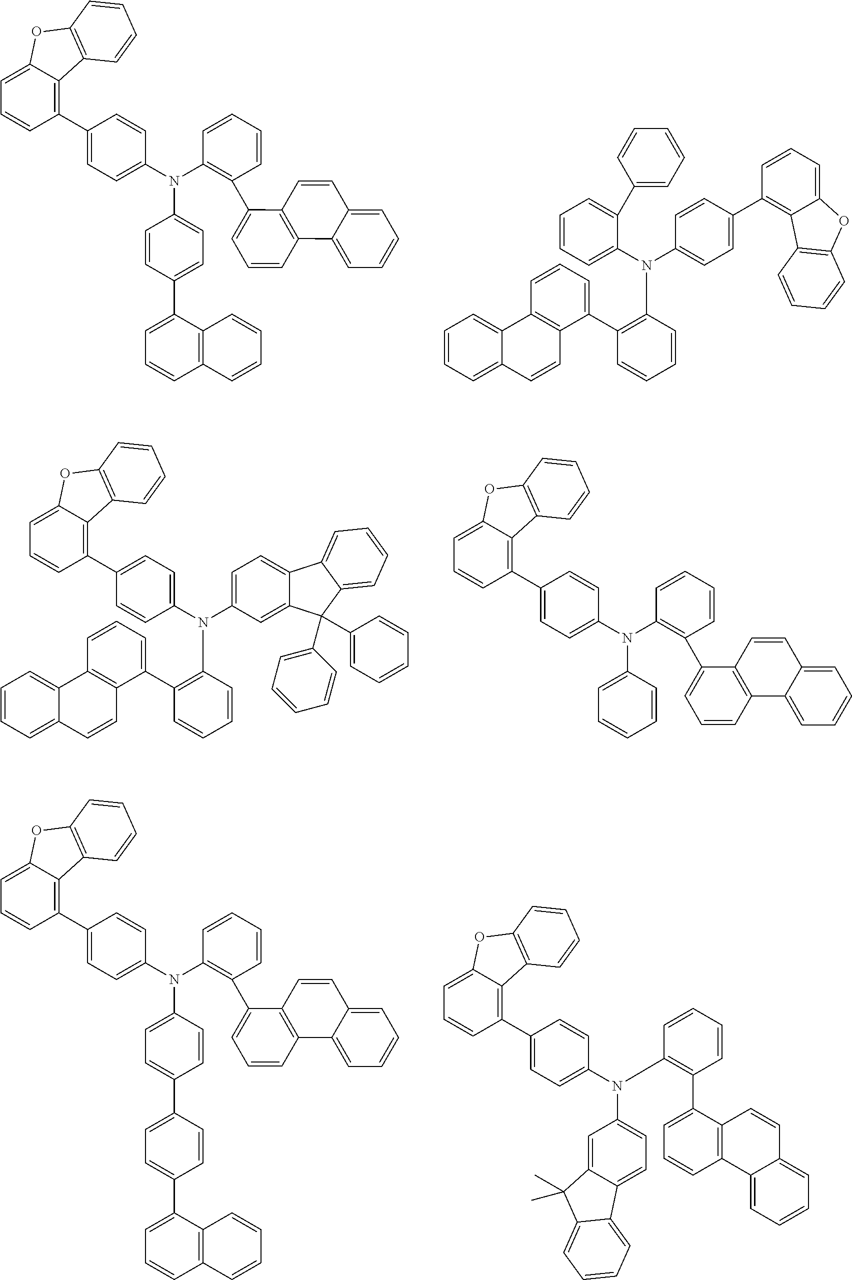

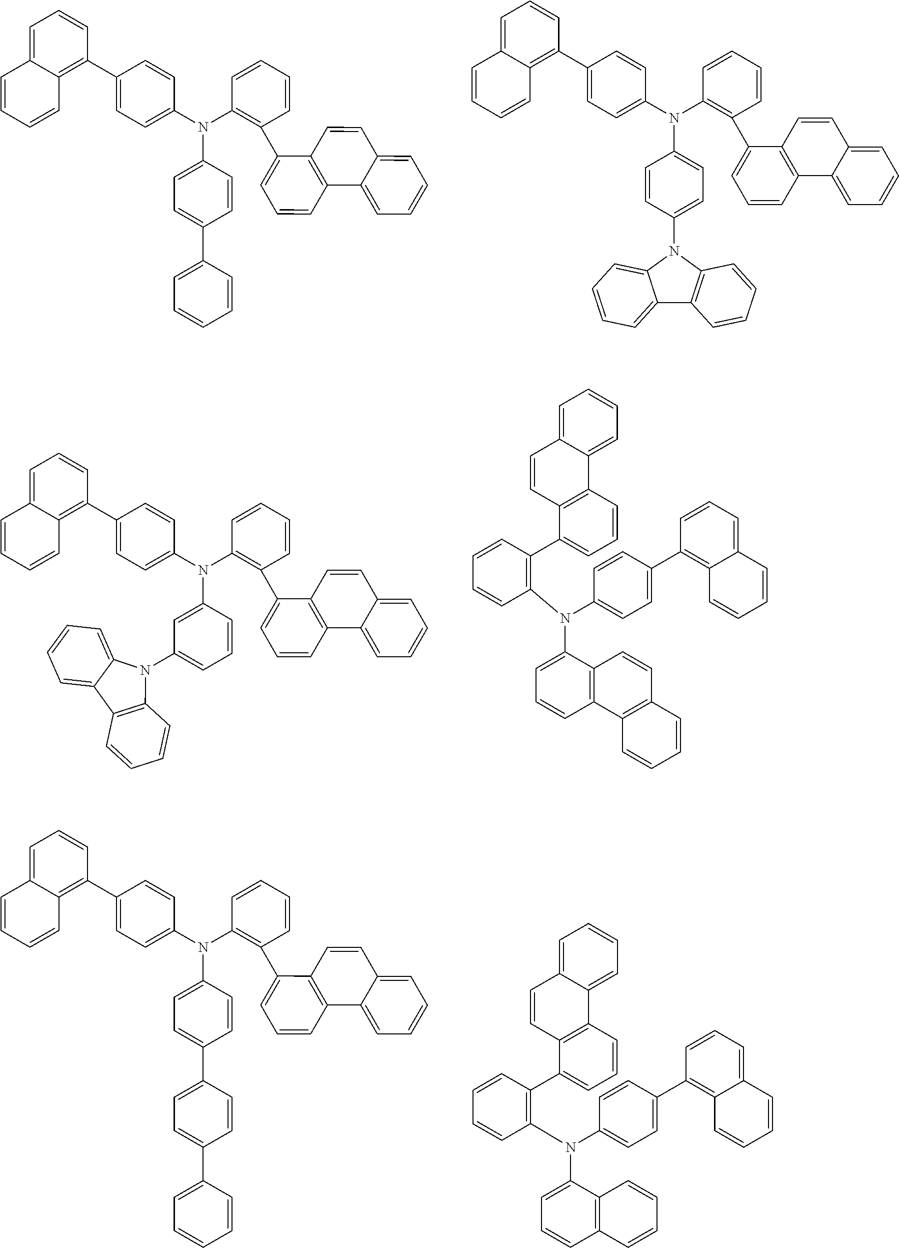

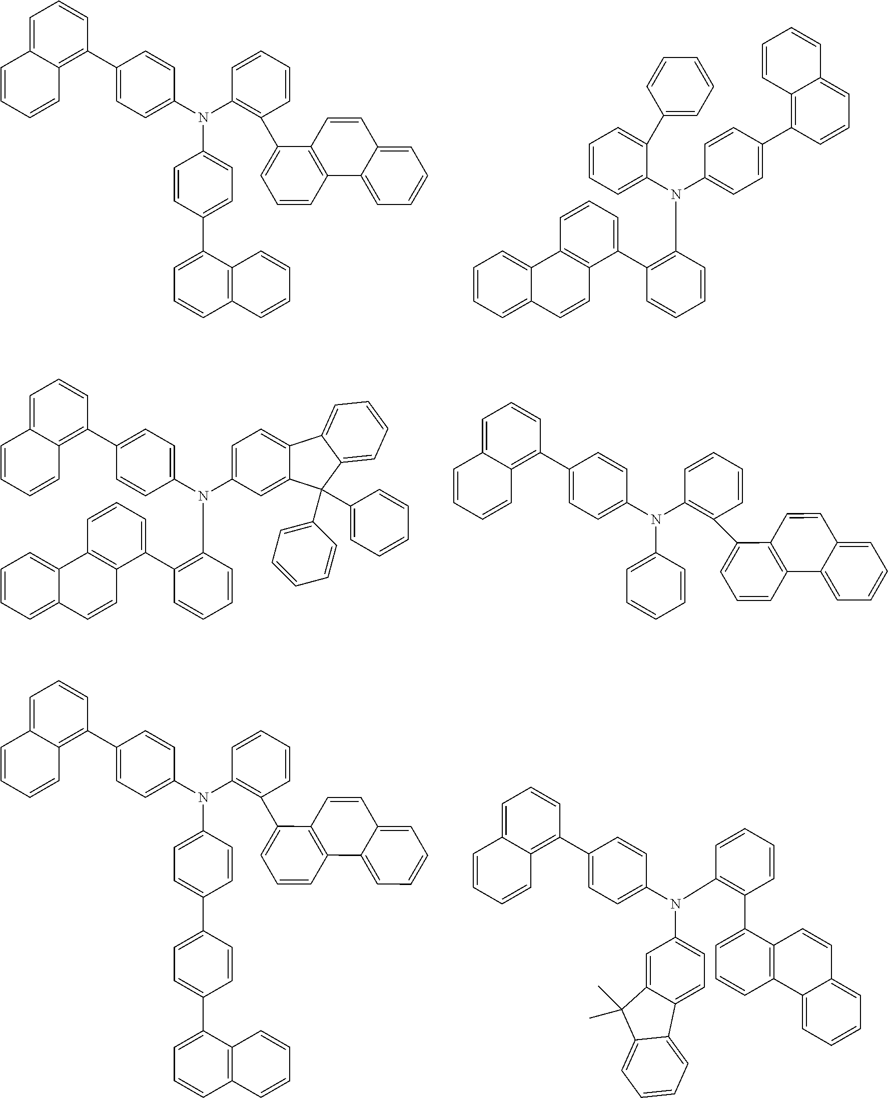

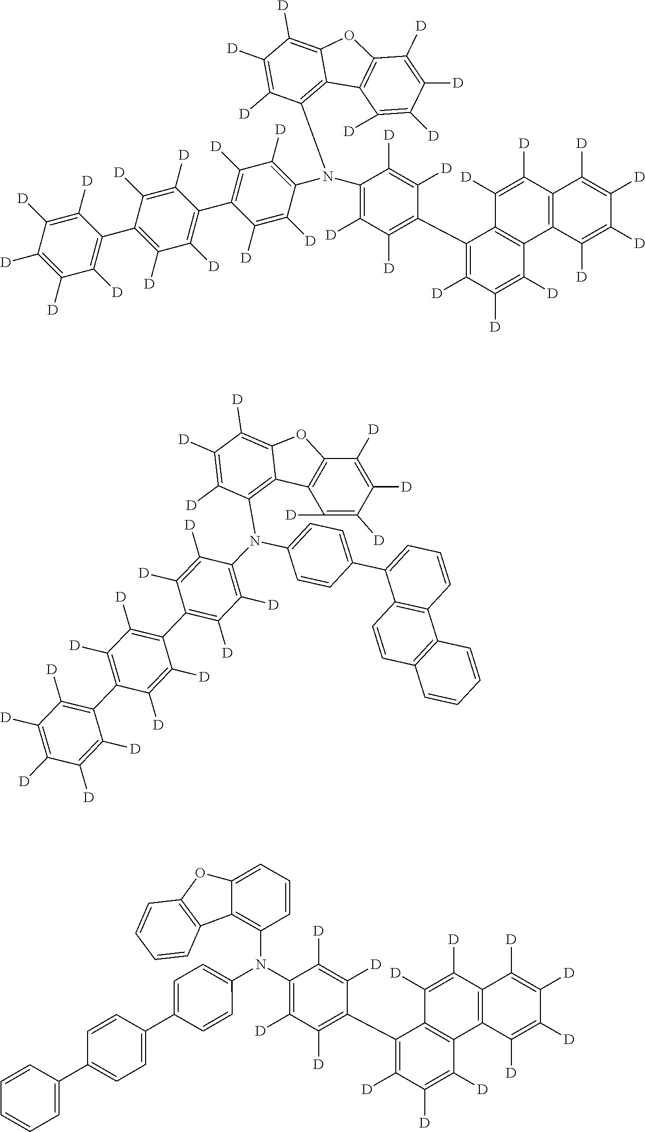

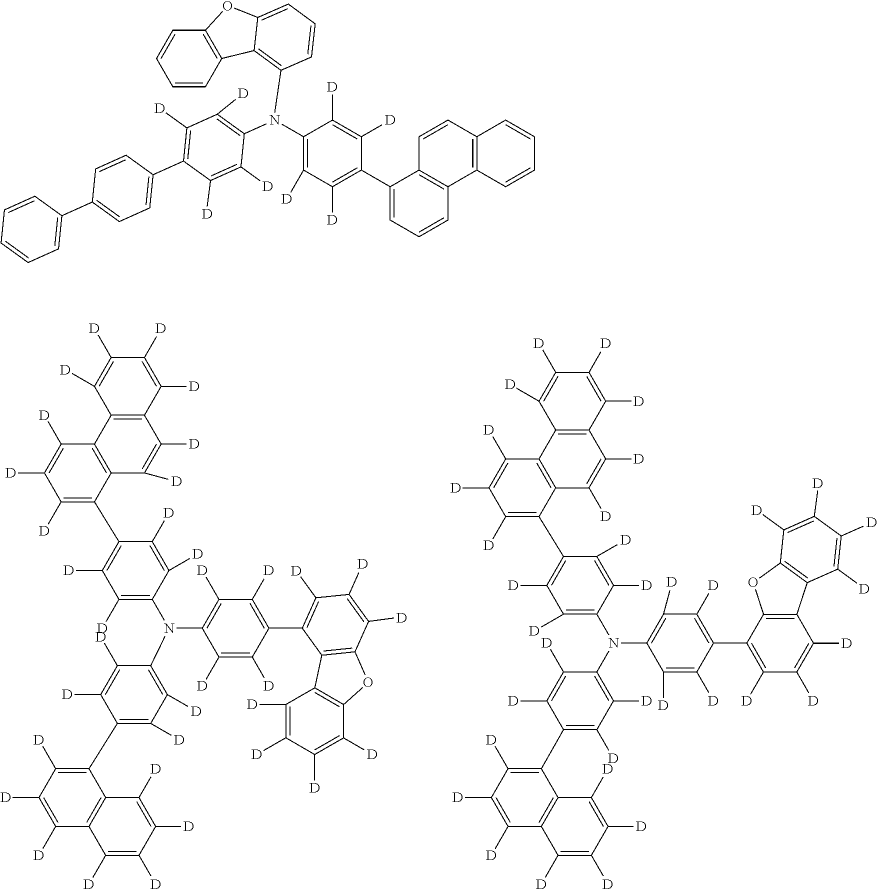

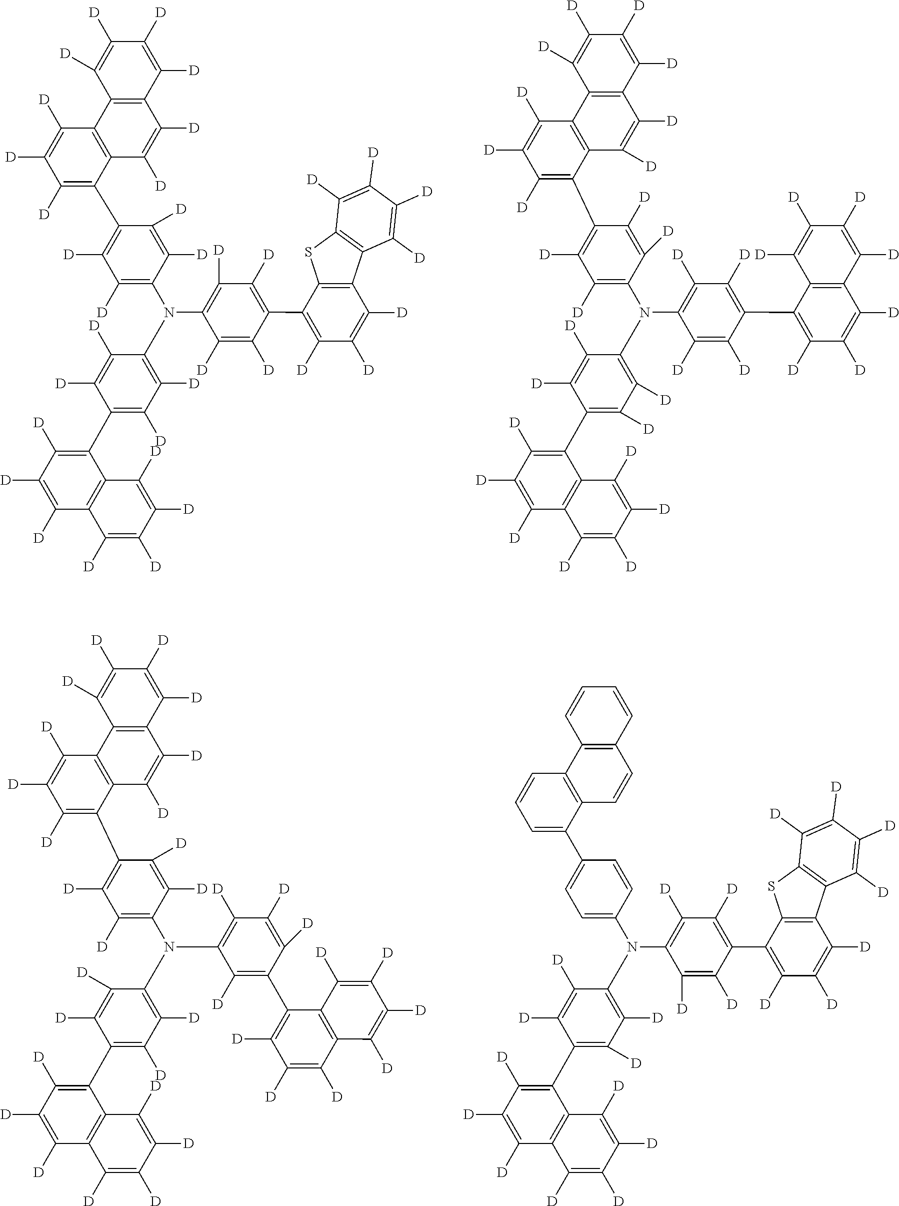

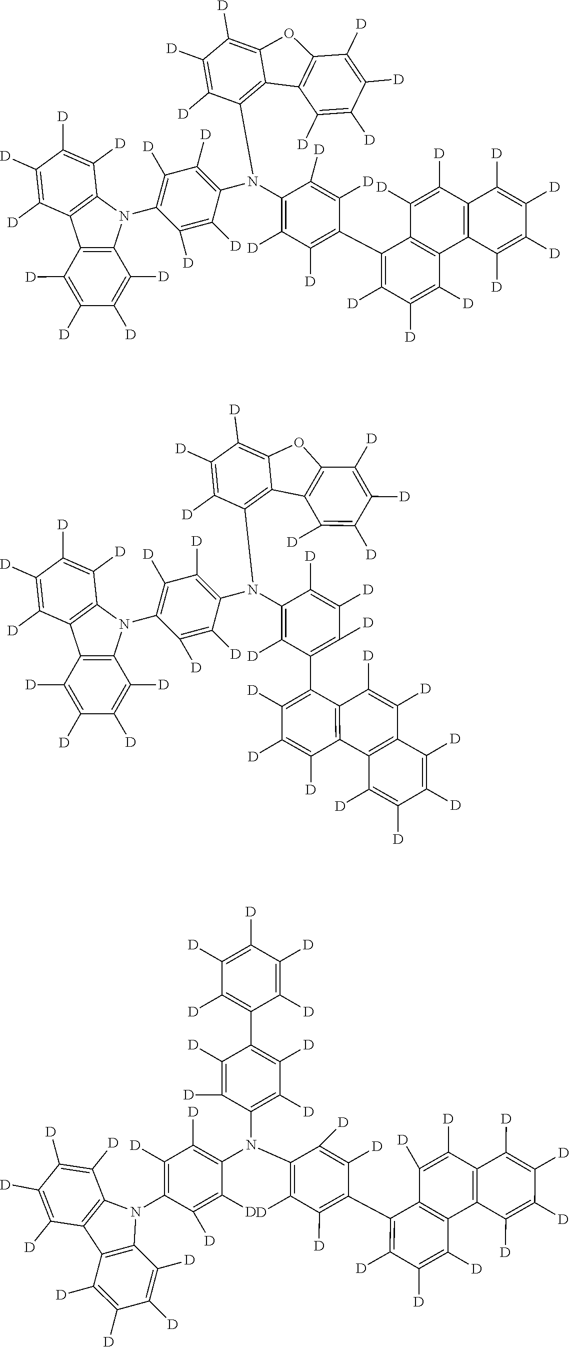

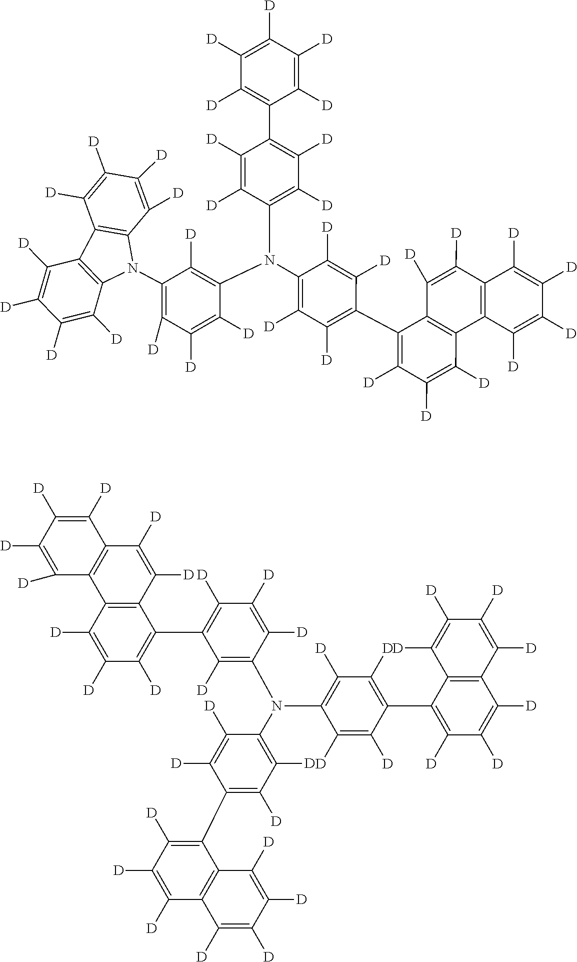

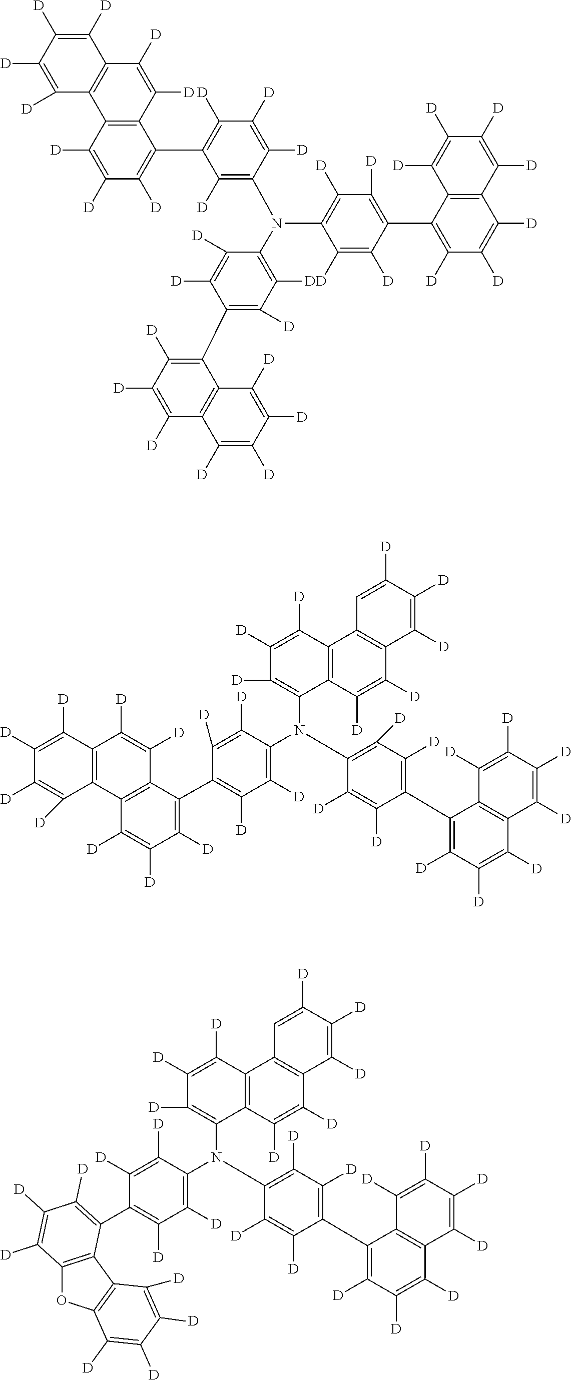

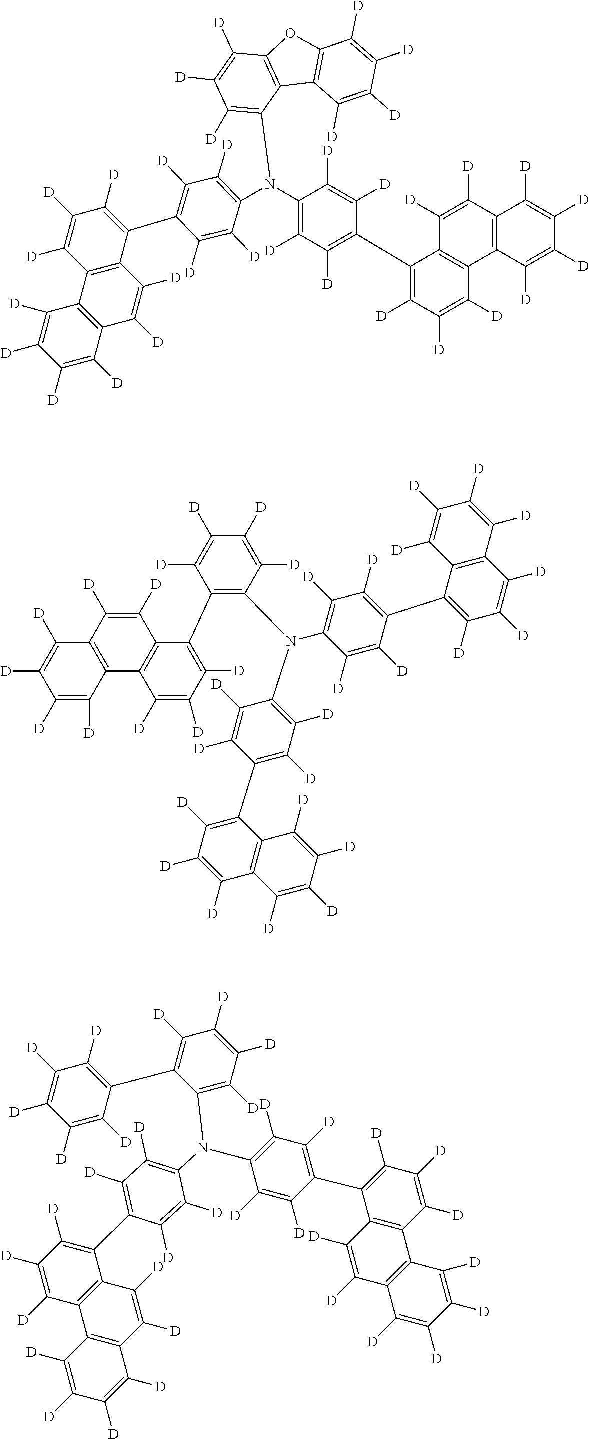

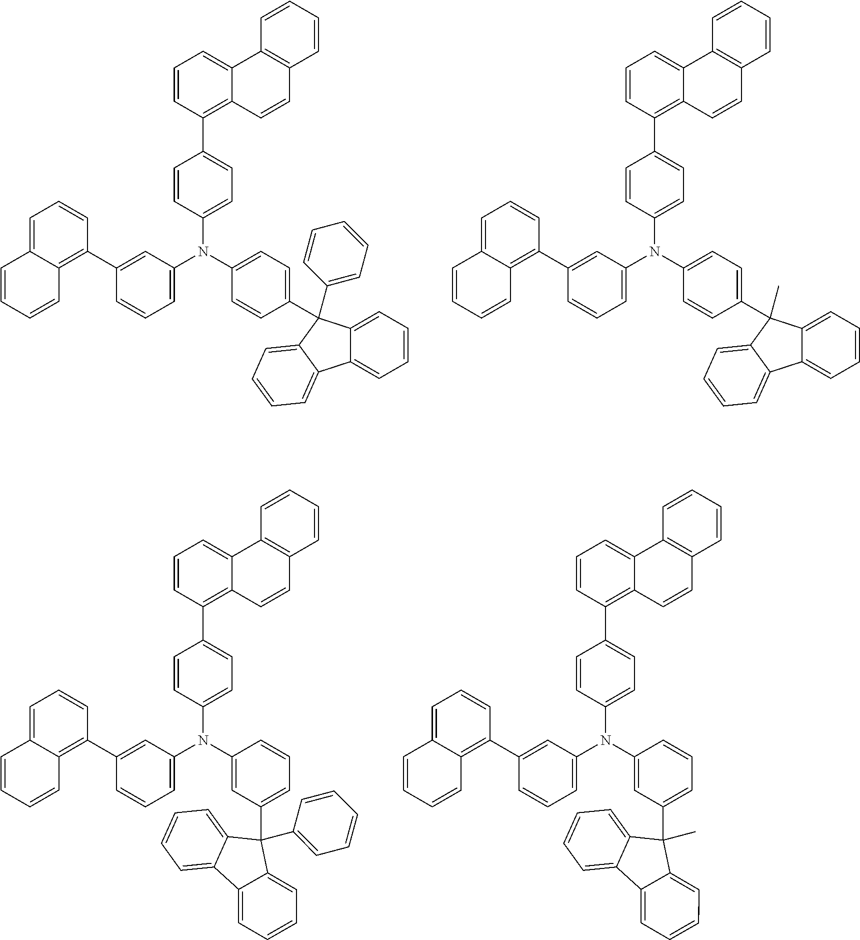 [SMILES: c1ccc(C2(c3ccc(N(c4ccc(-c5cccc6c5ccc5ccccc56)cc4)c4cccc(-c5cccc6ccccc56)c4)cc3)c3ccccc3-c3ccccc32)cc1 ; CC1(c2ccc(N(c3ccc(-c4cccc5c4ccc4ccccc45)cc3)c3cccc(-c4cccc5ccccc45)c3)cc2)c2ccccc2-c2ccccc21 ; c1ccc(C2(c3cccc(N(c4ccc(-c5cccc6c5ccc5ccccc56)cc4)c4cccc(-c5cccc6ccccc56)c4)c3)c3ccccc3-c3ccccc32)cc1 ; CC1(c2cccc(N(c3ccc(-c4cccc5c4ccc4ccccc45)cc3)c3cccc(-c4cccc5ccccc45)c3)c2)c2ccccc2-c2ccccc21]

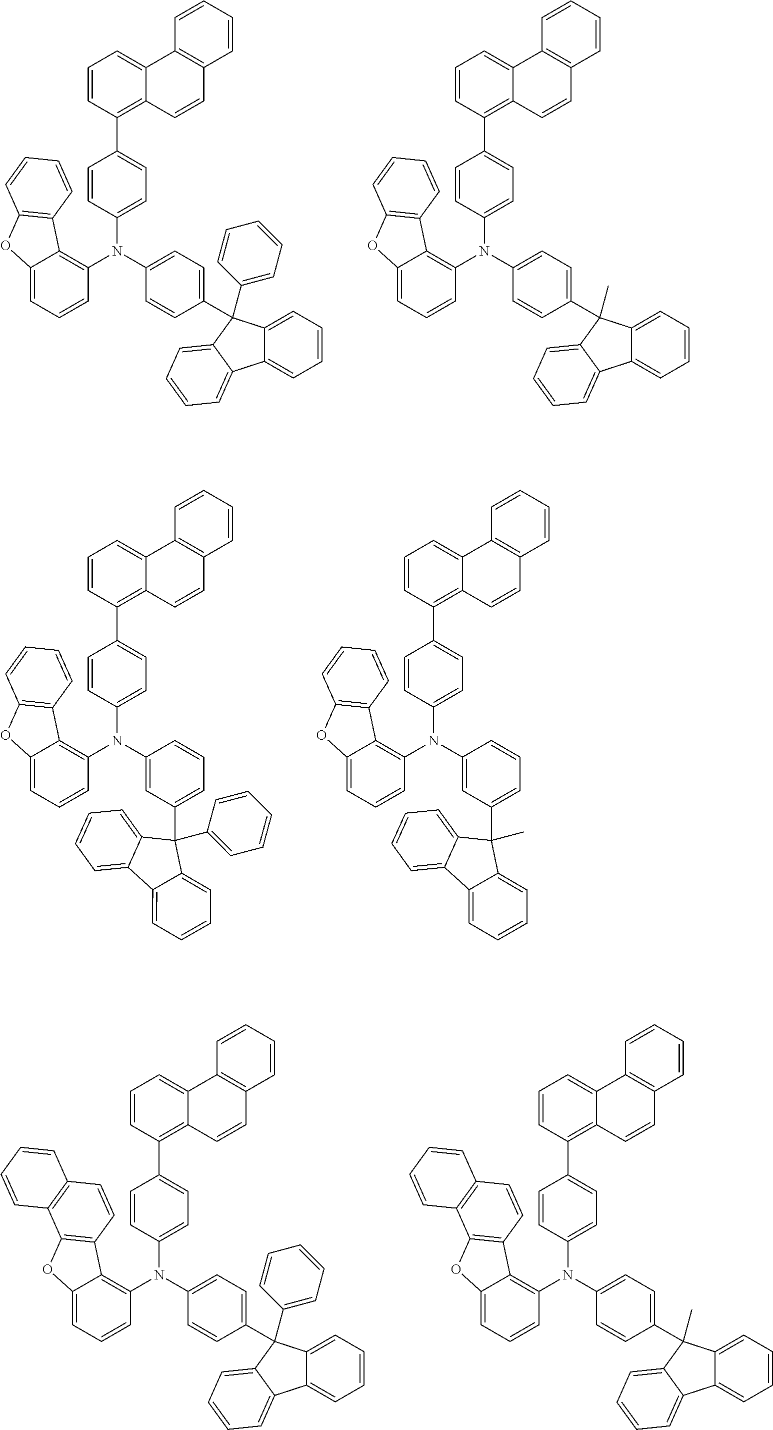

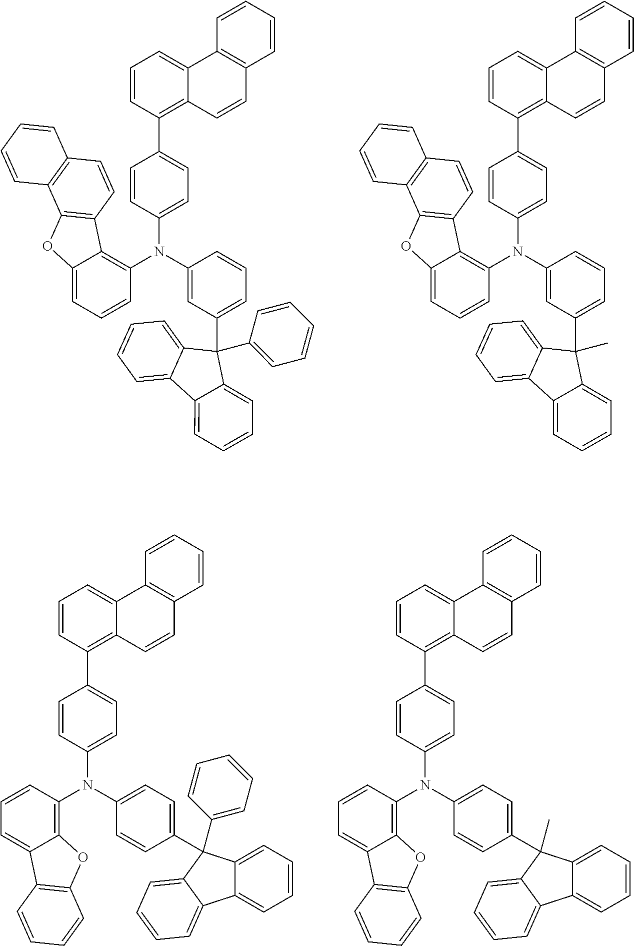 [SMILES: c1ccc(C2(c3cccc(N(c4ccc(-c5cccc6c5ccc5ccccc56)cc4)c4cccc5oc6c7ccccc7ccc6c45)c3)c3ccccc3-c3ccccc32)cc1 ; CC1(c2cccc(N(c3ccc(-c4cccc5c4ccc4ccccc45)cc3)c3cccc4oc5c6ccccc6ccc5c34)c2)c2ccccc2-c2ccccc21 ; c1ccc(C2(c3ccc(N(c4ccc(-c5cccc6c5ccc5ccccc56)cc4)c4cccc5c4oc4ccccc45)cc3)c3ccccc3-c3ccccc32)cc1 ; CC1(c2ccc(N(c3ccc(-c4cccc5c4ccc4ccccc45)cc3)c3cccc4c3oc3ccccc34)cc2)c2ccccc2-c2ccccc21]

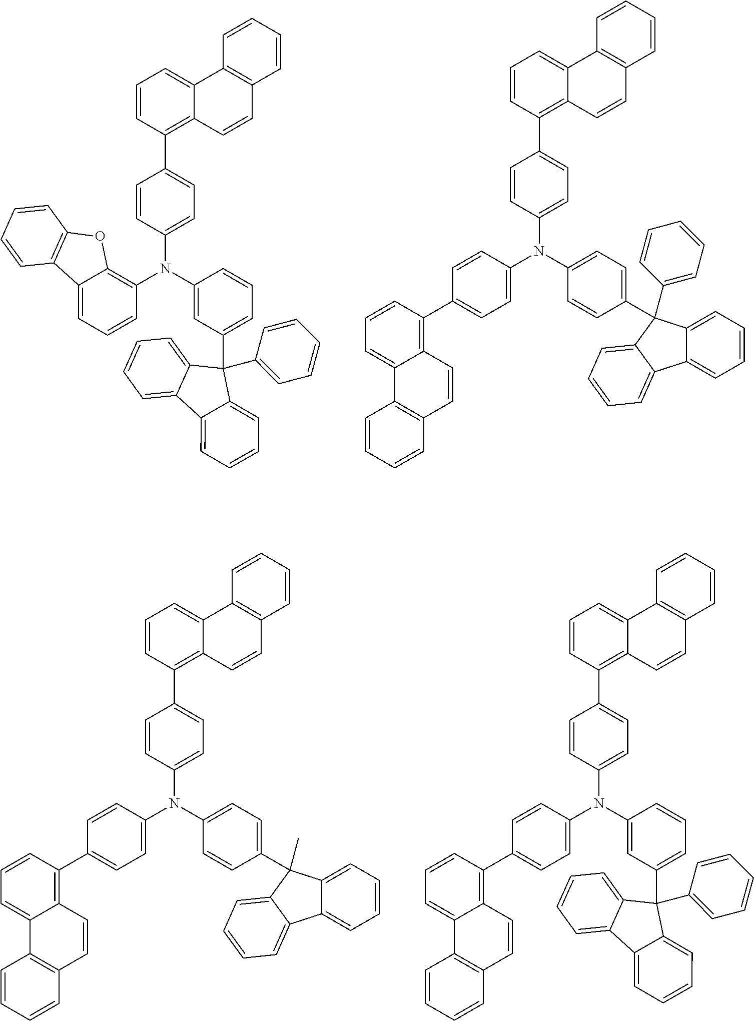 [SMILES: c1ccc(C2(c3cccc(N(c4ccc(-c5cccc6c5ccc5ccccc56)cc4)c4cccc5c4oc4ccccc45)c3)c3ccccc3-c3ccccc32)cc1 ; c1ccc(C2(c3ccc(N(c4ccc(-c5cccc6c5ccc5ccccc56)cc4)c4ccc(-c5cccc6c5ccc5ccccc56)cc4)cc3)c3ccccc3-c3ccccc32)cc1 ; CC1(c2ccc(N(c3ccc(-c4cccc5c4ccc4ccccc45)cc3)c3ccc(-c4cccc5c4ccc4ccccc45)cc3)cc2)c2ccccc2-c2ccccc21 ; c1ccc(C2(c3cccc(N(c4ccc(-c5cccc6c5ccc5ccccc56)cc4)c4ccc(-c5cccc6c5ccc5ccccc56)cc4)c3)c3ccccc3-c3ccccc32)cc1]

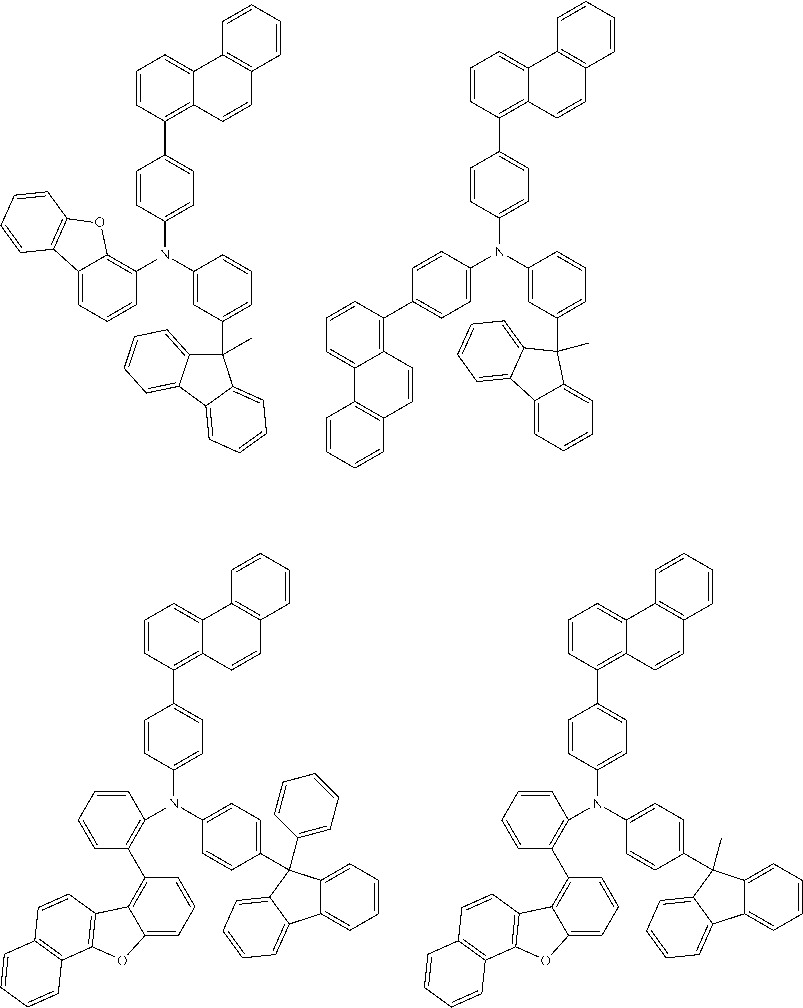 [SMILES: CC1(c2cccc(N(c3ccc(-c4cccc5c4ccc4ccccc45)cc3)c3cccc4c3oc3ccccc34)c2)c2ccccc2-c2ccccc21 ; CC1(c2cccc(N(c3ccc(-c4cccc5c4ccc4ccccc45)cc3)c3ccc(-c4cccc5c4ccc4ccccc45)cc3)c2)c2ccccc2-c2ccccc21 ; c1ccc(C2(c3ccc(N(c4ccc(-c5cccc6c5ccc5ccccc56)cc4)c4ccccc4-c4cccc5oc6c7ccccc7ccc6c45)cc3)c3ccccc3-c3ccccc32)cc1 ; CC1(c2ccc(N(c3ccc(-c4cccc5c4ccc4ccccc45)cc3)c3ccccc3-c3cccc4oc5c6ccccc6ccc5c34)cc2)c2ccccc2-c2ccccc21]

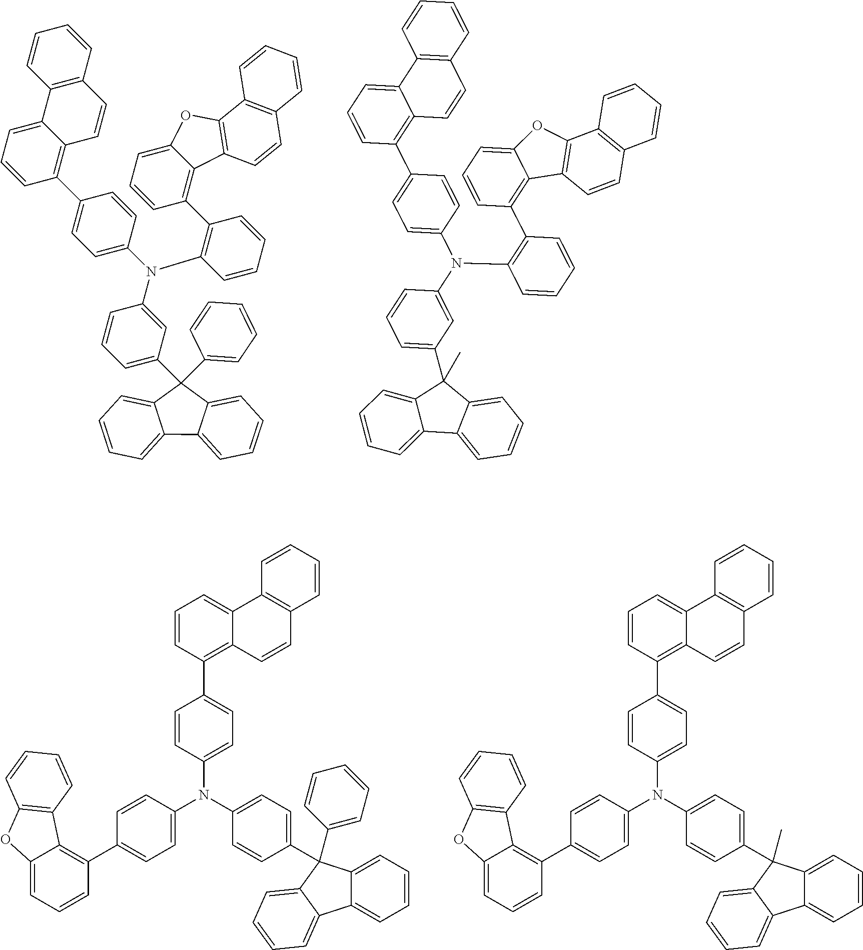 [SMILES: c1ccc(C2(c3cccc(N(c4ccc(-c5cccc6c5ccc5ccccc56)cc4)c4ccccc4-c4cccc5oc6c7ccccc7ccc6c45)c3)c3ccccc3-c3ccccc32)cc1 ; CC1(c2cccc(N(c3ccc(-c4cccc5c4ccc4ccccc45)cc3)c3ccccc3-c3cccc4oc5c6ccccc6ccc5c34)c2)c2ccccc2-c2ccccc21 ; c1ccc(C2(c3ccc(N(c4ccc(-c5cccc6c5ccc5ccccc56)cc4)c4ccc(-c5cccc6oc7ccccc7c56)cc4)cc3)c3ccccc3-c3ccccc32)cc1 ; CC1(c2ccc(N(c3ccc(-c4cccc5c4ccc4ccccc45)cc3)c3ccc(-c4cccc5oc6ccccc6c45)cc3)cc2)c2ccccc2-c2ccccc21]

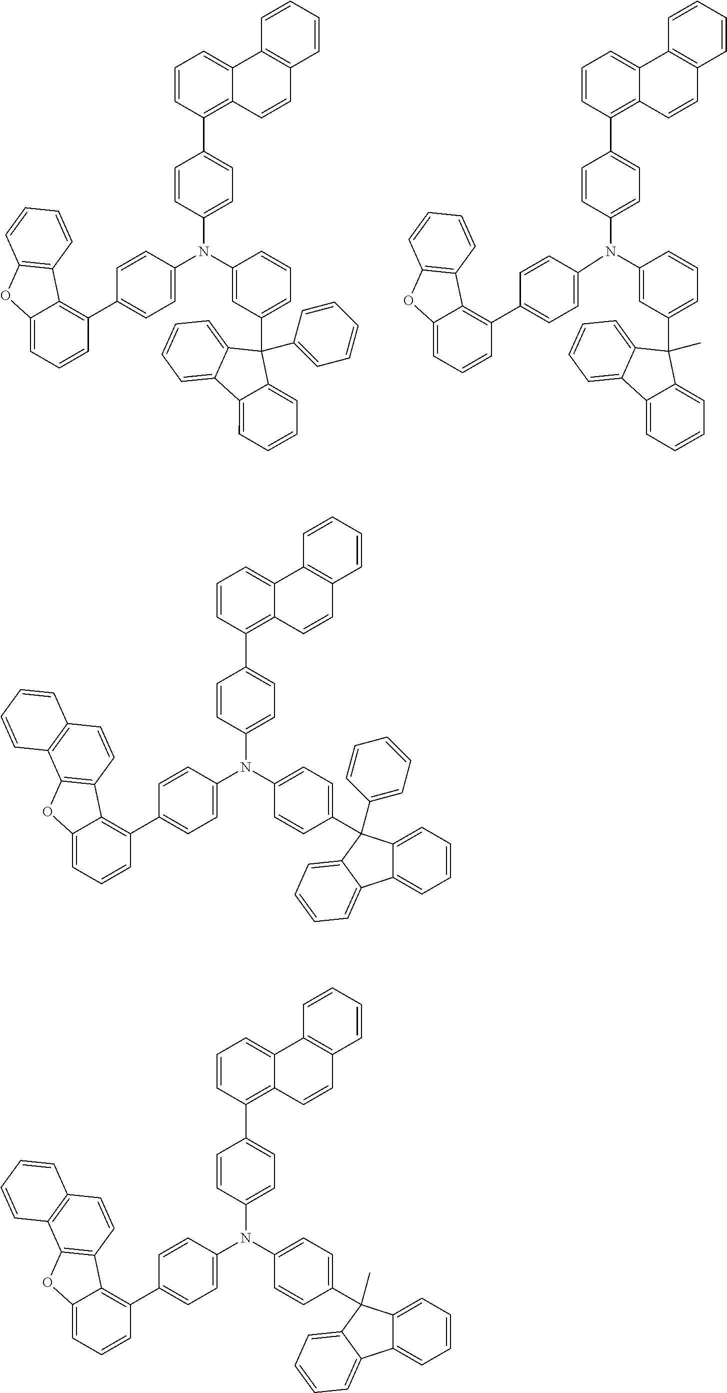

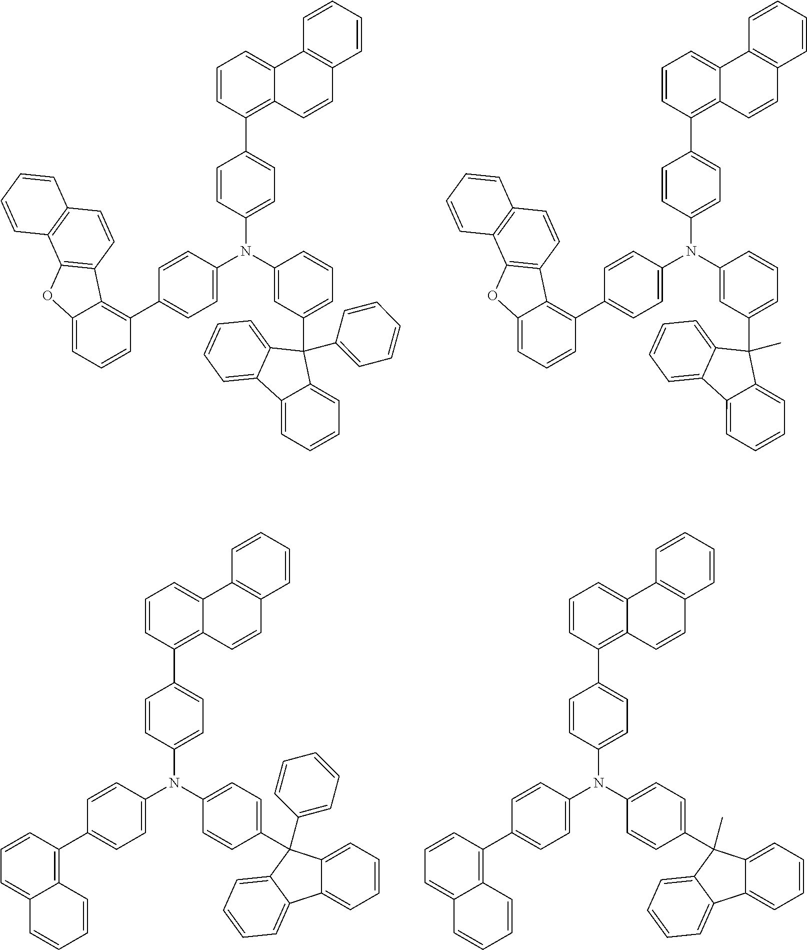 [SMILES: c1ccc(C2(c3cccc(N(c4ccc(-c5cccc6c5ccc5ccccc56)cc4)c4ccc(-c5cccc6oc7c8ccccc8ccc7c56)cc4)c3)c3ccccc3-c3ccccc32)cc1 ; CC1(c2cccc(N(c3ccc(-c4cccc5c4ccc4ccccc45)cc3)c3ccc(-c4cccc5oc6c7ccccc7ccc6c45)cc3)c2)c2ccccc2-c2ccccc21 ; c1ccc(C2(c3ccc(N(c4ccc(-c5cccc6ccccc56)cc4)c4ccc(-c5cccc6c5ccc5ccccc56)cc4)cc3)c3ccccc3-c3ccccc32)cc1 ; CC1(c2ccc(N(c3ccc(-c4cccc5ccccc45)cc3)c3ccc(-c4cccc5c4ccc4ccccc45)cc3)cc2)c2ccccc2-c2ccccc21]

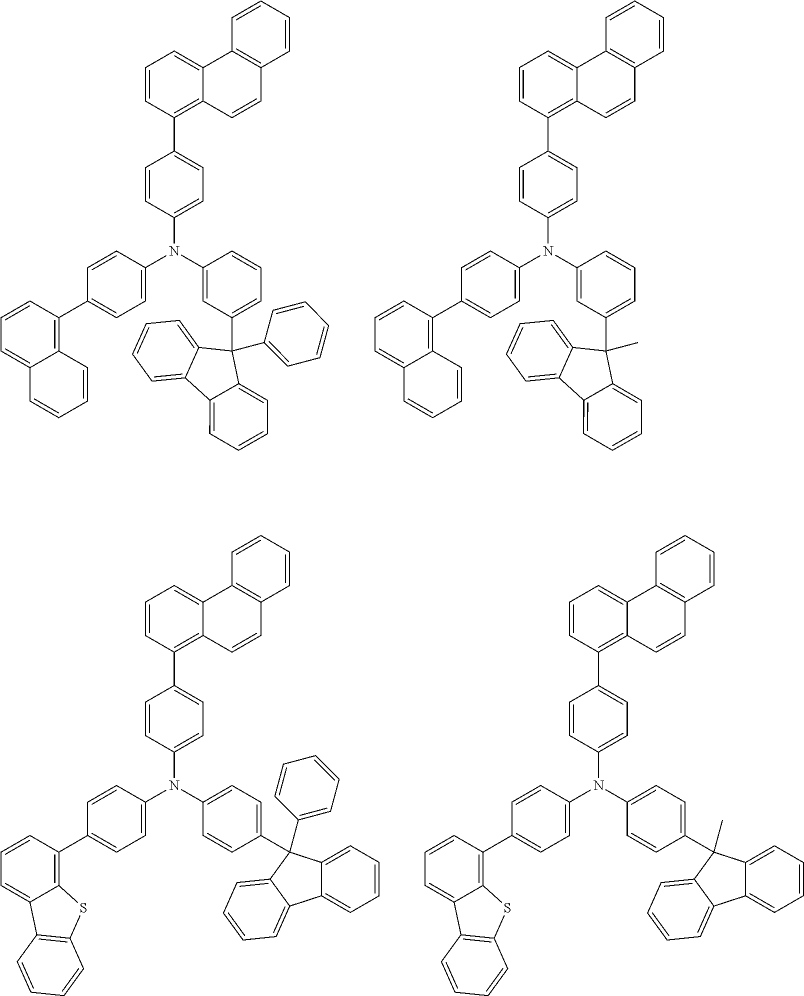 [SMILES: c1ccc(C2(c3cccc(N(c4ccc(-c5cccc6ccccc56)cc4)c4ccc(-c5cccc6c5ccc5ccccc56)cc4)c3)c3ccccc3-c3ccccc32)cc1 ; CC1(c2cccc(N(c3ccc(-c4cccc5ccccc45)cc3)c3ccc(-c4cccc5c4ccc4ccccc45)cc3)c2)c2ccccc2-c2ccccc21 ; c1ccc(C2(c3ccc(N(c4ccc(-c5cccc6c5ccc5ccccc56)cc4)c4ccc(-c5cccc6c5sc5ccccc56)cc4)cc3)c3ccccc3-c3ccccc32)cc1 ; CC1(c2ccc(N(c3ccc(-c4cccc5c4ccc4ccccc45)cc3)c3ccc(-c4cccc5c4sc4ccccc45)cc3)cc2)c2ccccc2-c2ccccc21]

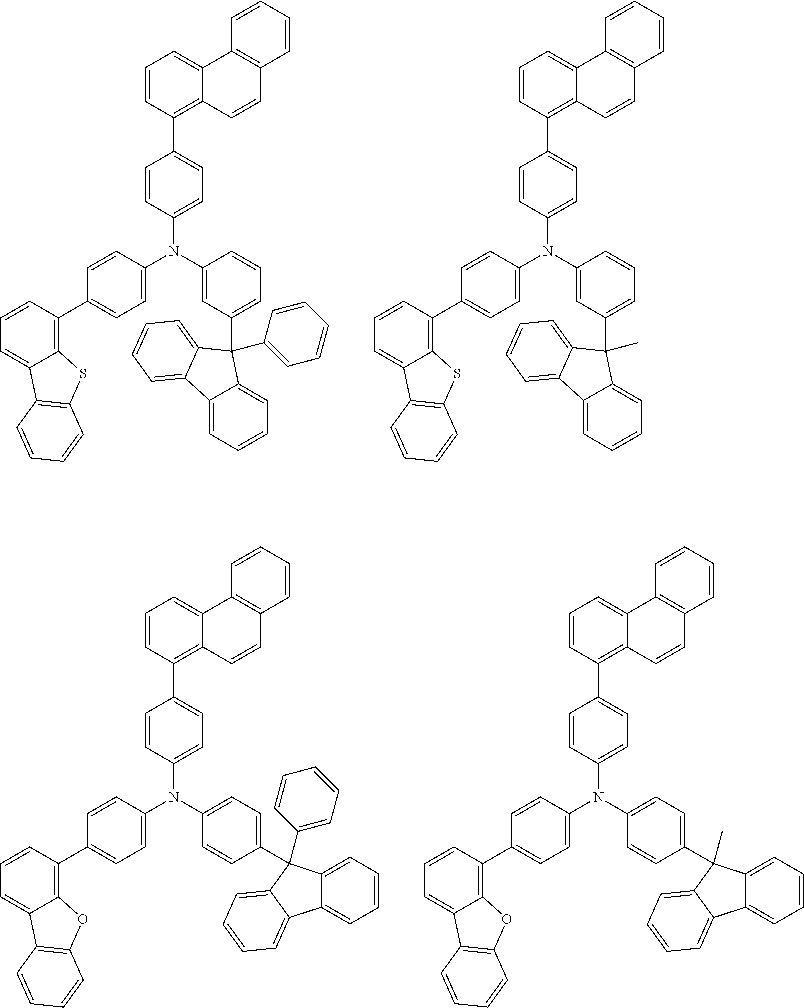 [SMILES: c1ccc(C2(c3cccc(N(c4ccc(-c5cccc6c5ccc5ccccc56)cc4)c4ccc(-c5cccc6c5sc5ccccc56)cc4)c3)c3ccccc3-c3ccccc32)cc1 ; CC1(c2cccc(N(c3ccc(-c4cccc5c4ccc4ccccc45)cc3)c3ccc(-c4cccc5c4sc4ccccc45)cc3)c2)c2ccccc2-c2ccccc21 ; c1ccc(C2(c3ccc(N(c4ccc(-c5cccc6c5ccc5ccccc56)cc4)c4ccc(-c5cccc6c5oc5ccccc56)cc4)cc3)c3ccccc3-c3ccccc32)cc1 ; CC1(c2ccc(N(c3ccc(-c4cccc5c4ccc4ccccc45)cc3)c3ccc(-c4cccc5c4oc4ccccc45)cc3)cc2)c2ccccc2-c2ccccc21]

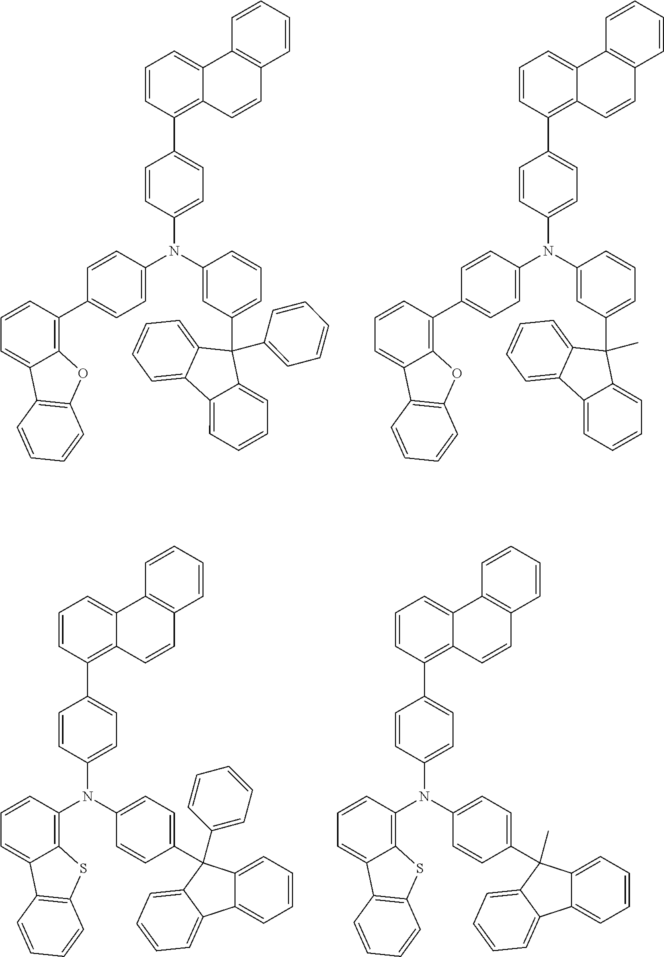 [SMILES: c1ccc(C2(c3cccc(N(c4ccc(-c5cccc6c5ccc5ccccc56)cc4)c4ccc(-c5cccc6c5oc5ccccc56)cc4)c3)c3ccccc3-c3ccccc32)cc1 ; CC1(c2cccc(N(c3ccc(-c4cccc5c4ccc4ccccc45)cc3)c3ccc(-c4cccc5c4oc4ccccc45)cc3)c2)c2ccccc2-c2ccccc21 ; c1ccc(C2(c3ccc(N(c4ccc(-c5cccc6c5ccc5ccccc56)cc4)c4cccc5c4sc4ccccc45)cc3)c3ccccc3-c3ccccc32)cc1 ; CC1(c2ccc(N(c3ccc(-c4cccc5c4ccc4ccccc45)cc3)c3cccc4c3sc3ccccc34)cc2)c2ccccc2-c2ccccc21]

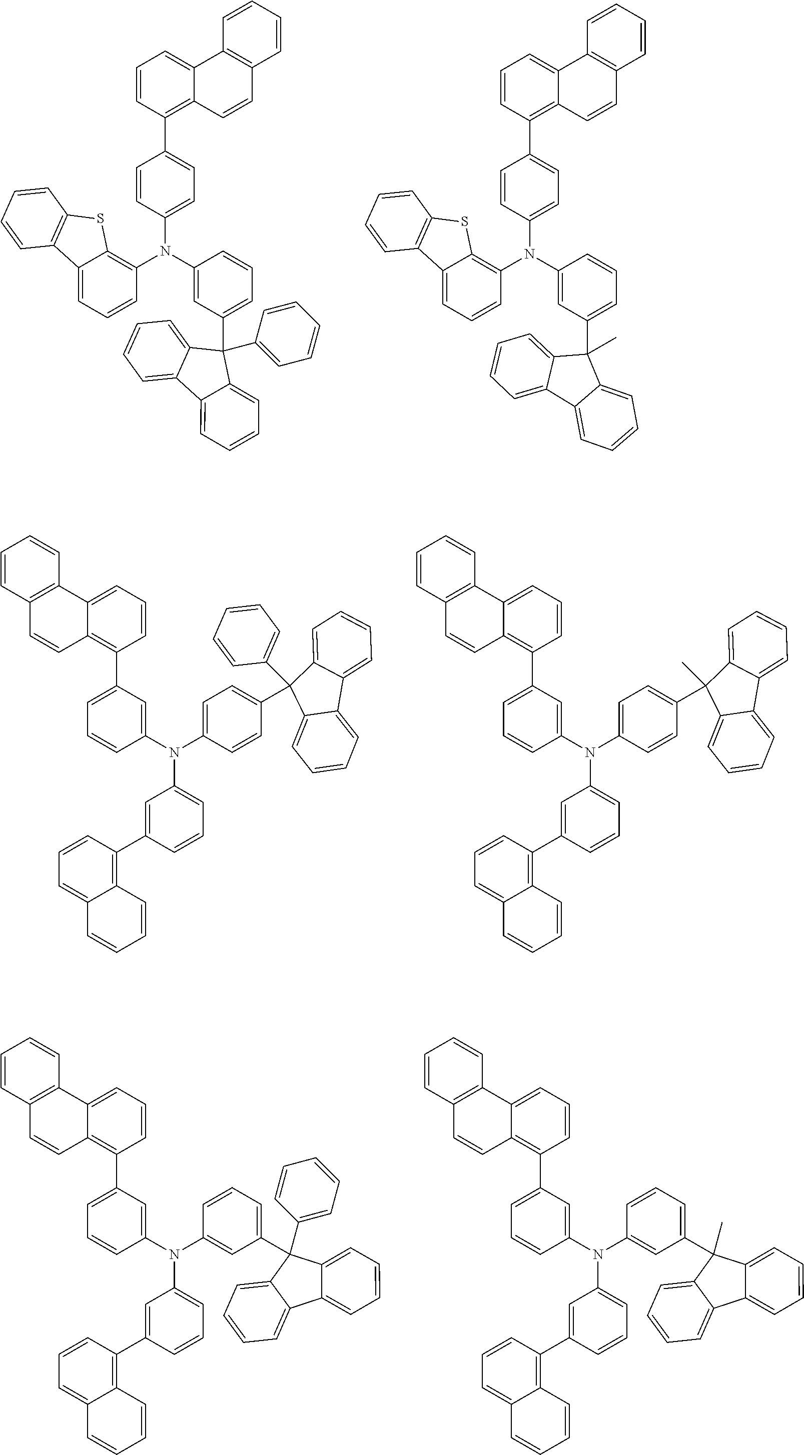

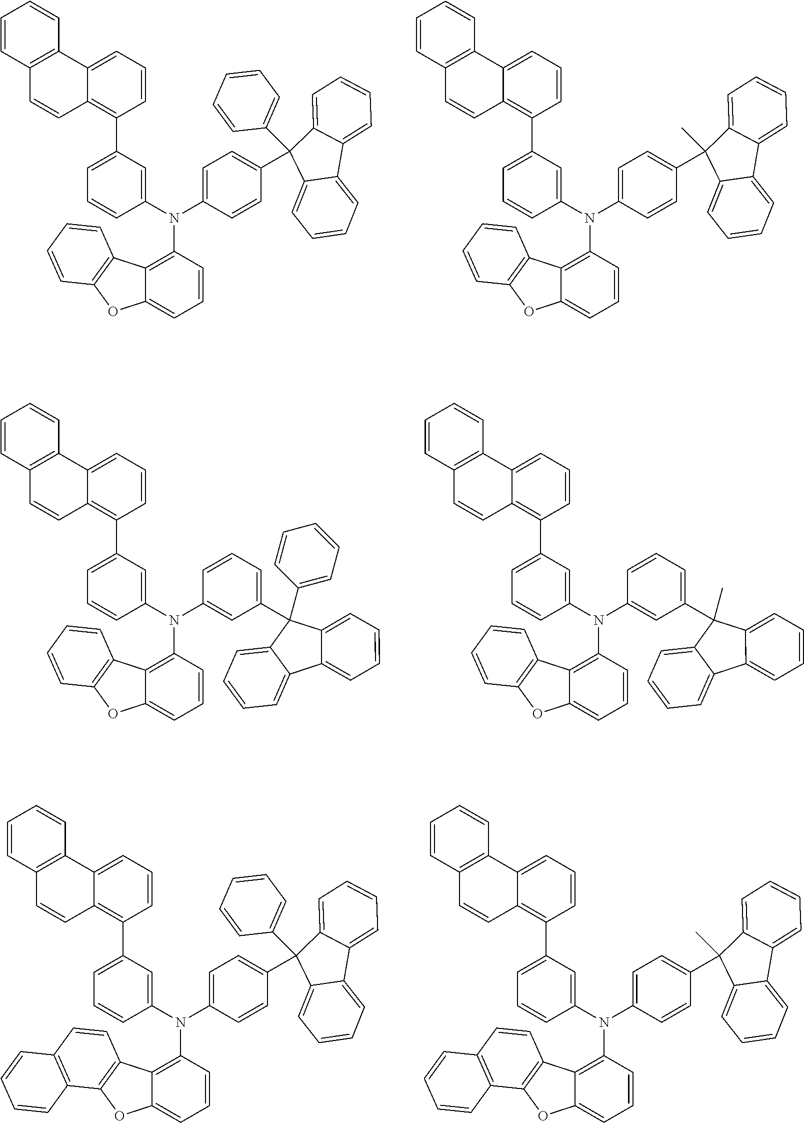

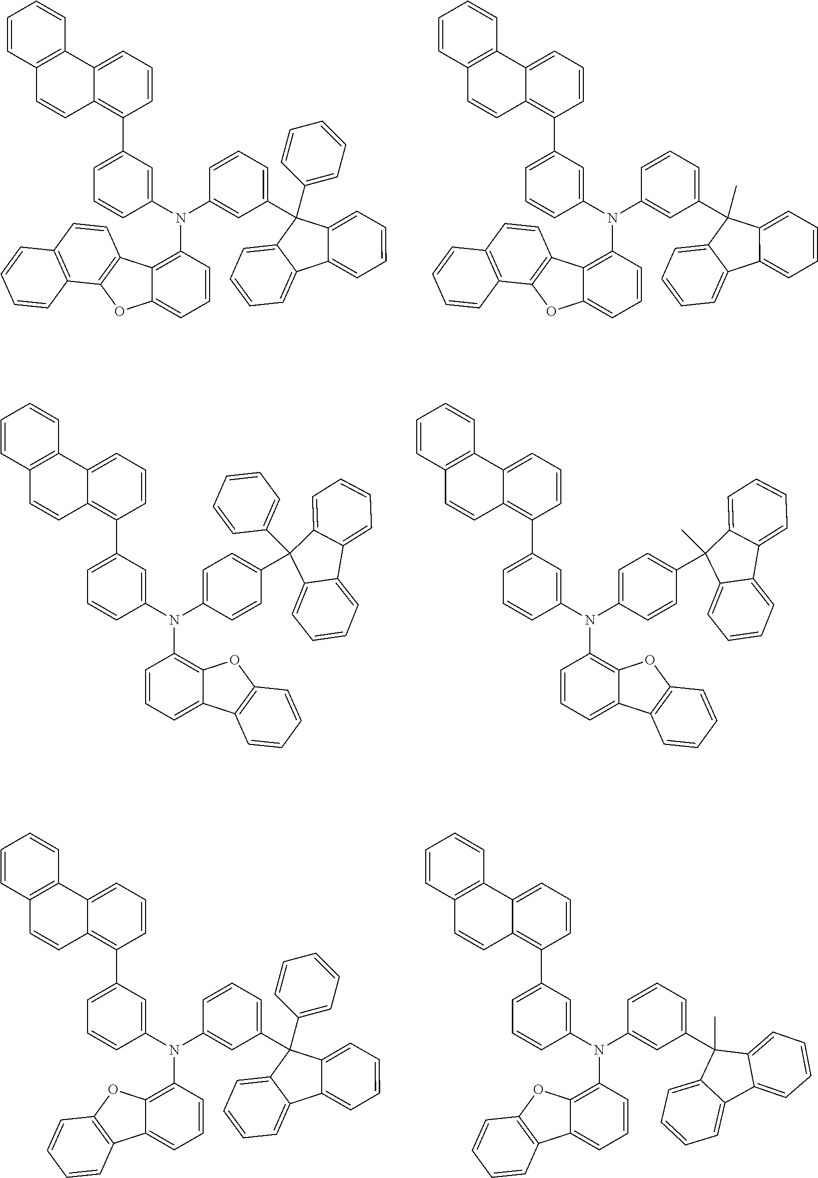

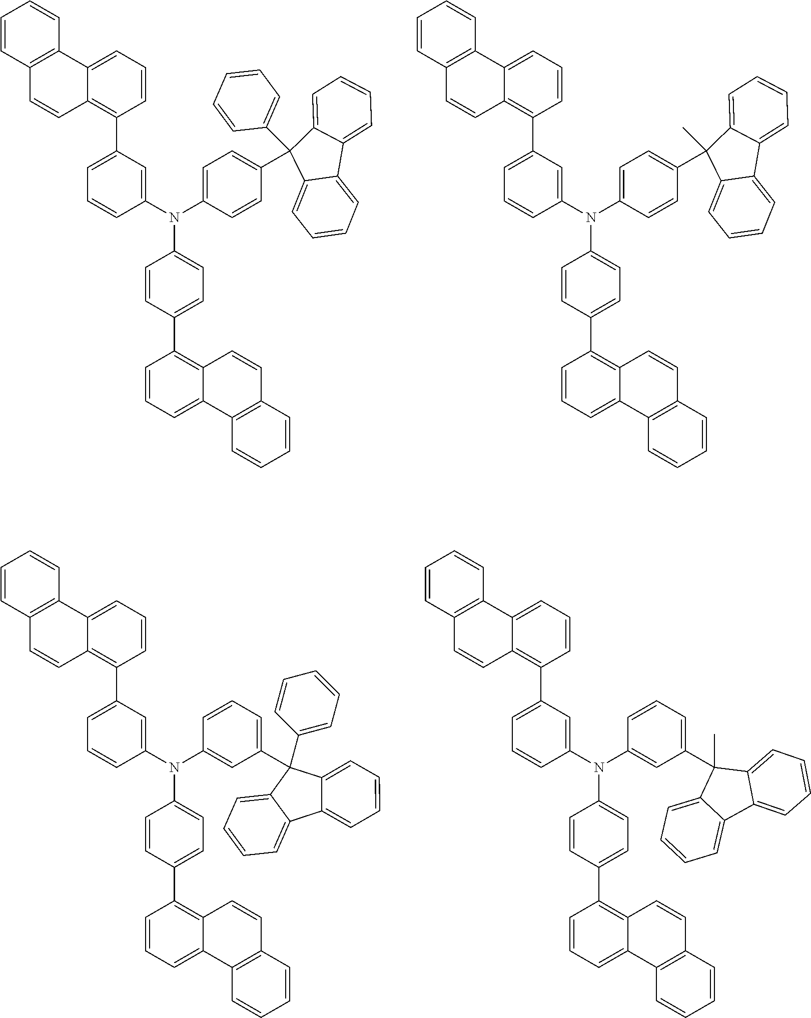 [SMILES: c1ccc(C2(c3ccc(N(c4ccc(-c5cccc6c5ccc5ccccc56)cc4)c4cccc(-c5cccc6c5ccc5ccccc56)c4)cc3)c3ccccc3-c3ccccc32)cc1 ; CC1(c2ccc(N(c3ccc(-c4cccc5c4ccc4ccccc45)cc3)c3cccc(-c4cccc5c4ccc4ccccc45)c3)cc2)c2ccccc2-c2ccccc21 ; c1ccc(C2(c3cccc(N(c4ccc(-c5cccc6c5ccc5ccccc56)cc4)c4cccc(-c5cccc6c5ccc5ccccc56)c4)c3)c3ccccc3-c3ccccc32)cc1 ; CC1(c2cccc(N(c3ccc(-c4cccc5c4ccc4ccccc45)cc3)c3cccc(-c4cccc5c4ccc4ccccc45)c3)c2)c2ccccc2-c2ccccc21]

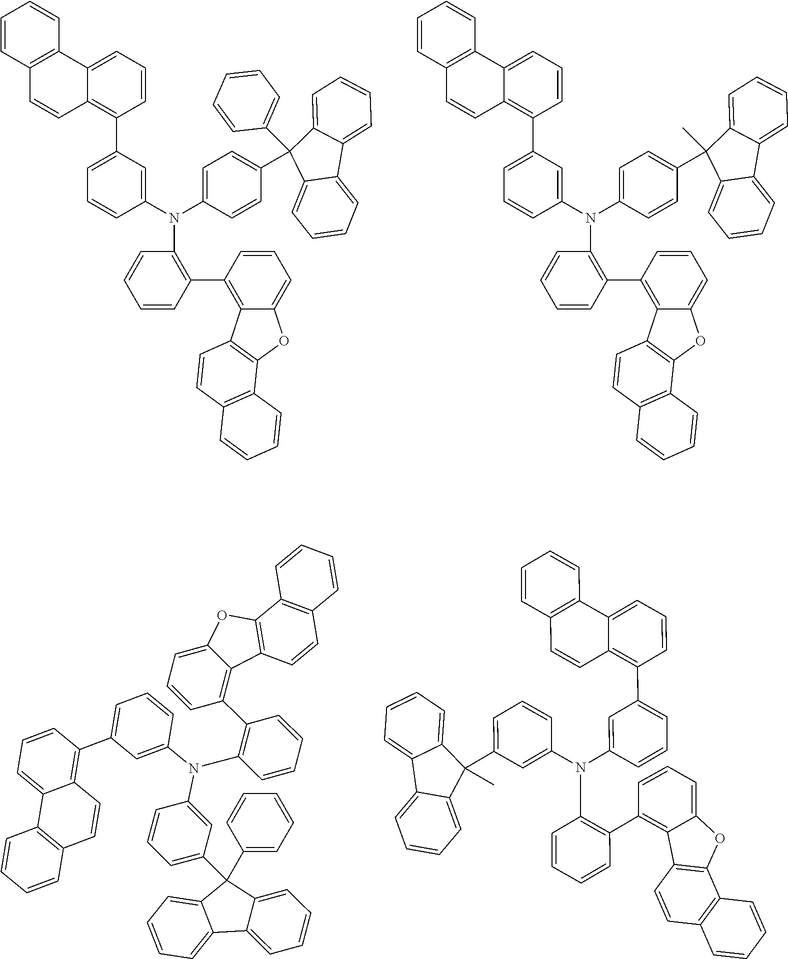 [SMILES: c1ccc(C2(c3ccc(N(c4cccc(-c5cccc6c5ccc5ccccc56)c4)c4ccccc4-c4cccc5oc6c7ccccc7ccc6c45)cc3)c3ccccc3-c3ccccc32)cc1 ; CC1(c2ccc(N(c3cccc(-c4cccc5c4ccc4ccccc45)c3)c3ccccc3-c3cccc4oc5c6ccccc6ccc5c34)cc2)c2ccccc2-c2ccccc21 ; c1ccc(C2(c3cccc(N(c4cccc(-c5cccc6c5ccc5ccccc56)c4)c4ccccc4-c4cccc5oc6c7ccccc7ccc6c45)c3)c3ccccc3-c3ccccc32)cc1 ; CC1(c2cccc(N(c3cccc(-c4cccc5c4ccc4ccccc45)c3)c3ccccc3-c3cccc4oc5c6ccccc6ccc5c34)c2)c2ccccc2-c2ccccc21]

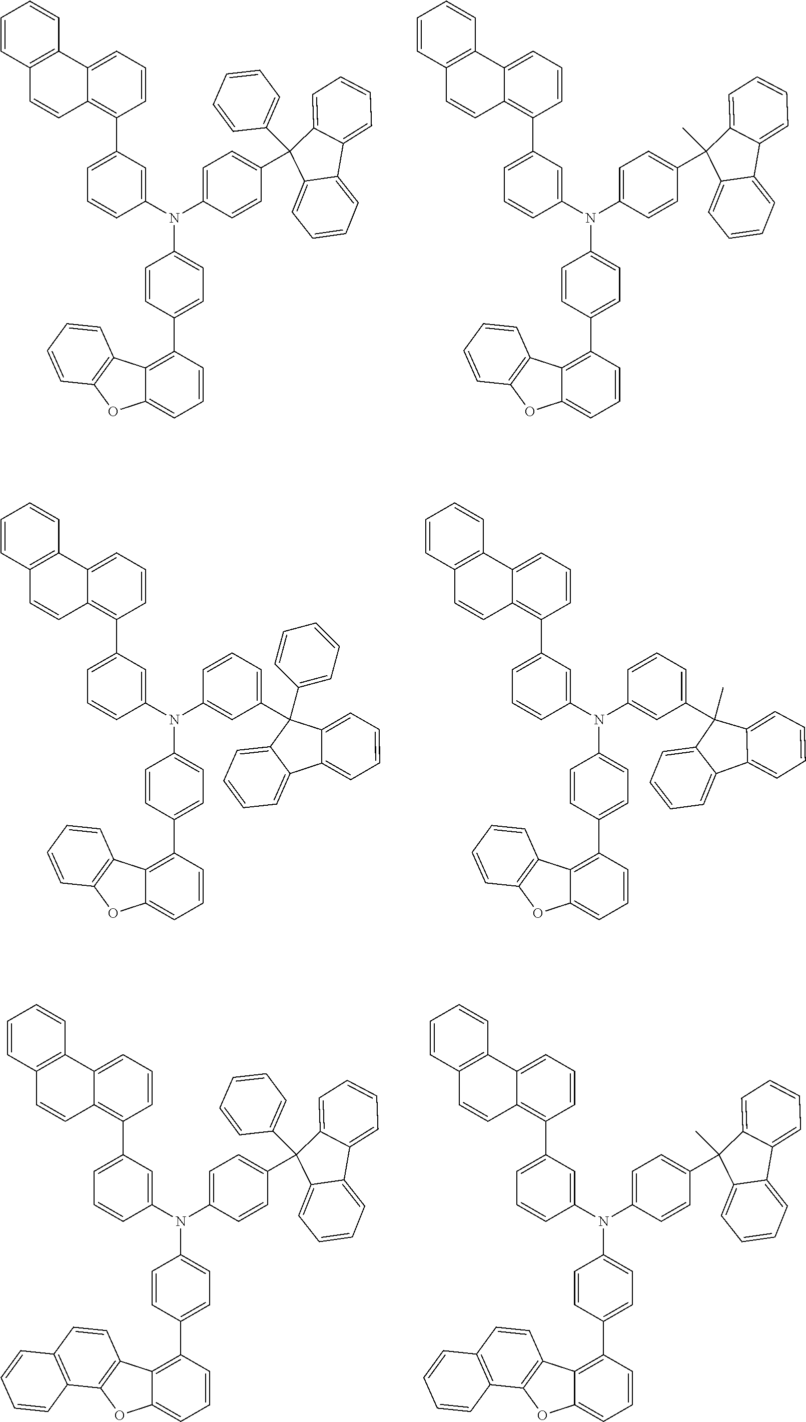

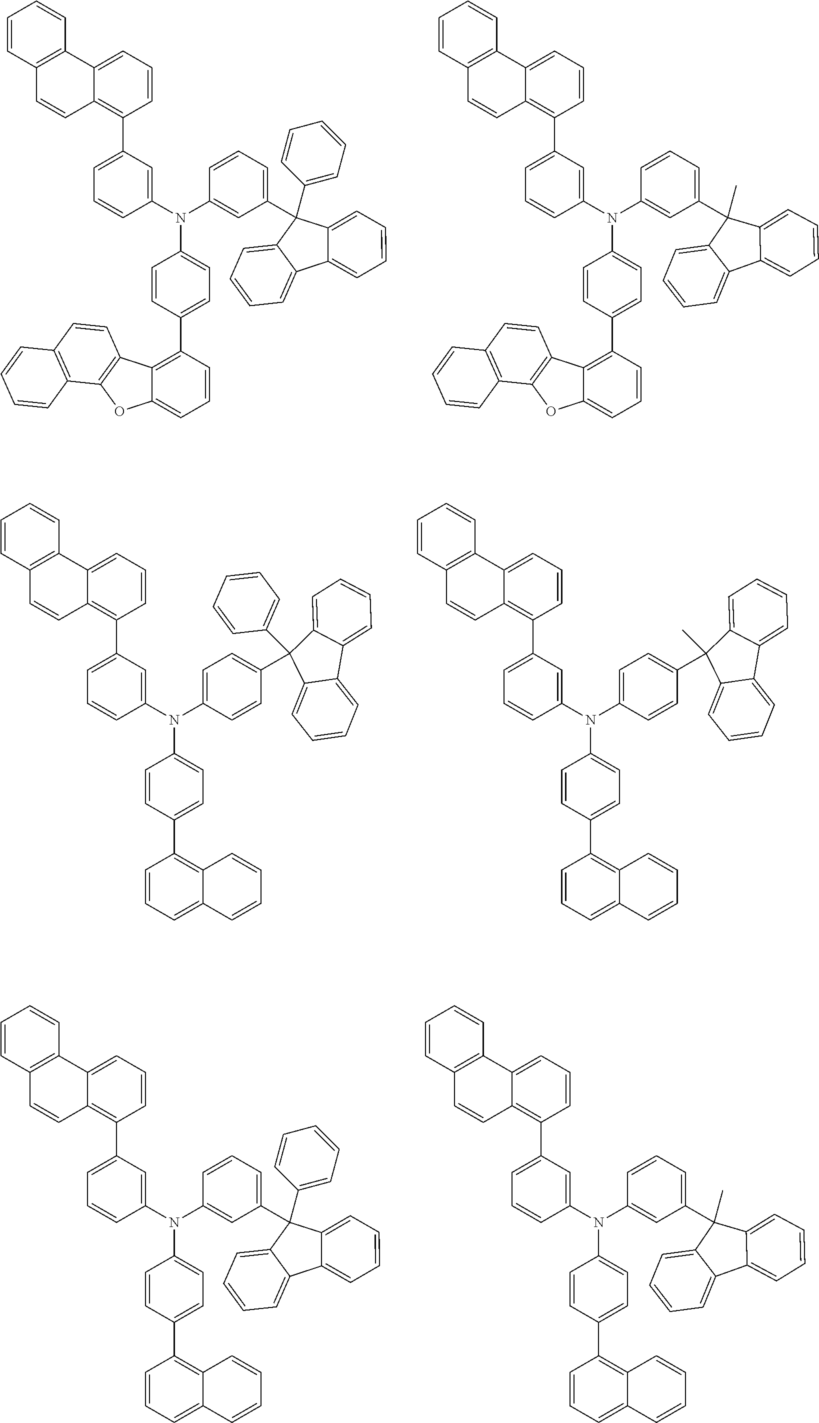

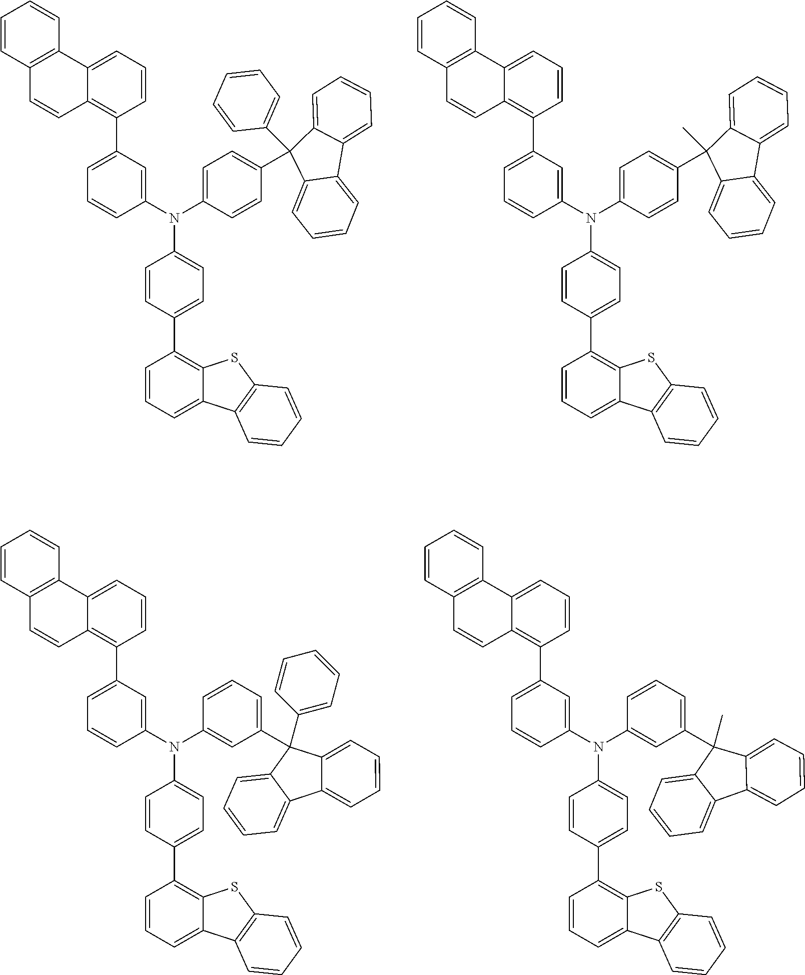 [SMILES: c1ccc(C2(c3ccc(N(c4ccc(-c5cccc6c5sc5ccccc56)cc4)c4cccc(-c5cccc6c5ccc5ccccc56)c4)cc3)c3ccccc3-c3ccccc32)cc1 ; CC1(c2ccc(N(c3ccc(-c4cccc5c4sc4ccccc45)cc3)c3cccc(-c4cccc5c4ccc4ccccc45)c3)cc2)c2ccccc2-c2ccccc21 ; c1ccc(C2(c3cccc(N(c4ccc(-c5cccc6c5sc5ccccc56)cc4)c4cccc(-c5cccc6c5ccc5ccccc56)c4)c3)c3ccccc3-c3ccccc32)cc1 ; CC1(c2cccc(N(c3ccc(-c4cccc5c4sc4ccccc45)cc3)c3cccc(-c4cccc5c4ccc4ccccc45)c3)c2)c2ccccc2-c2ccccc21]

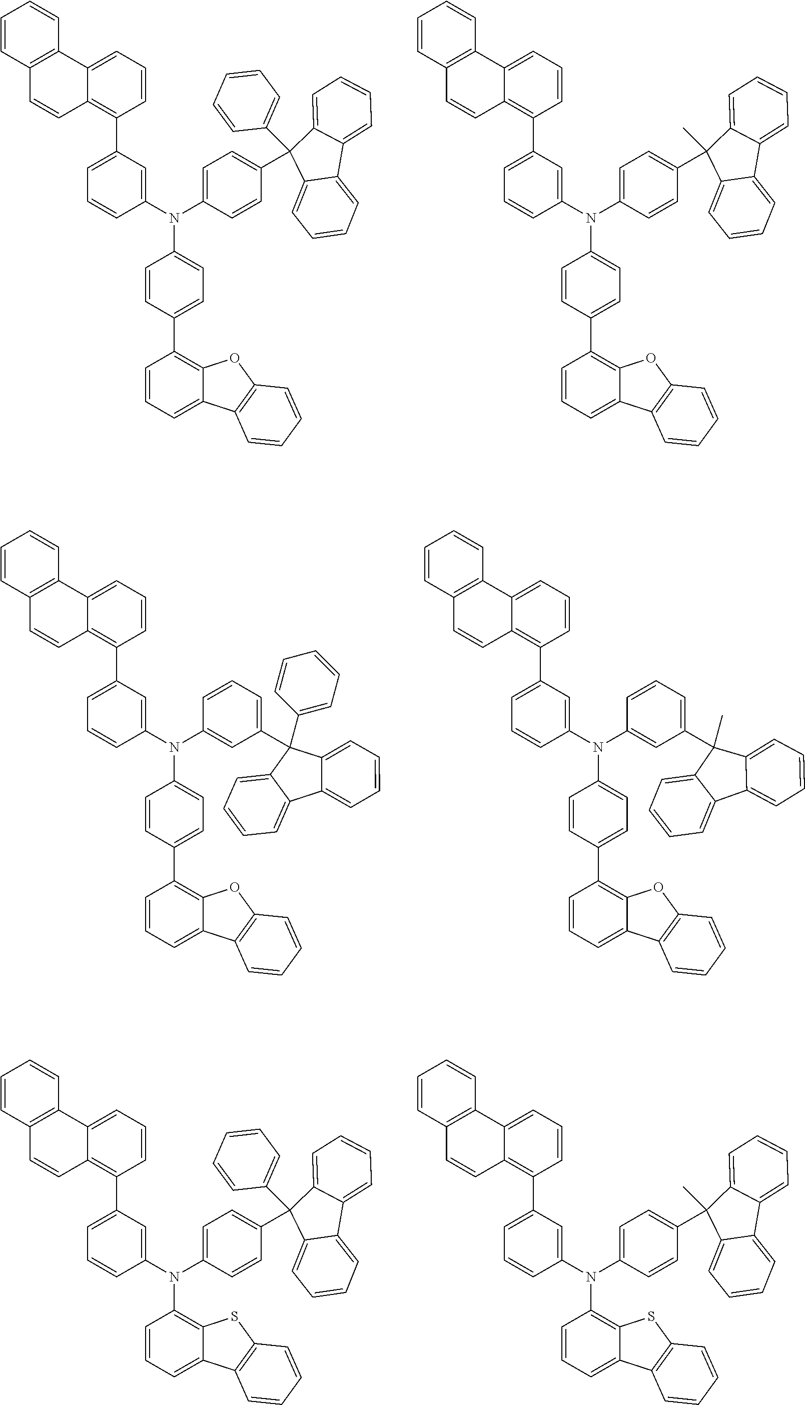

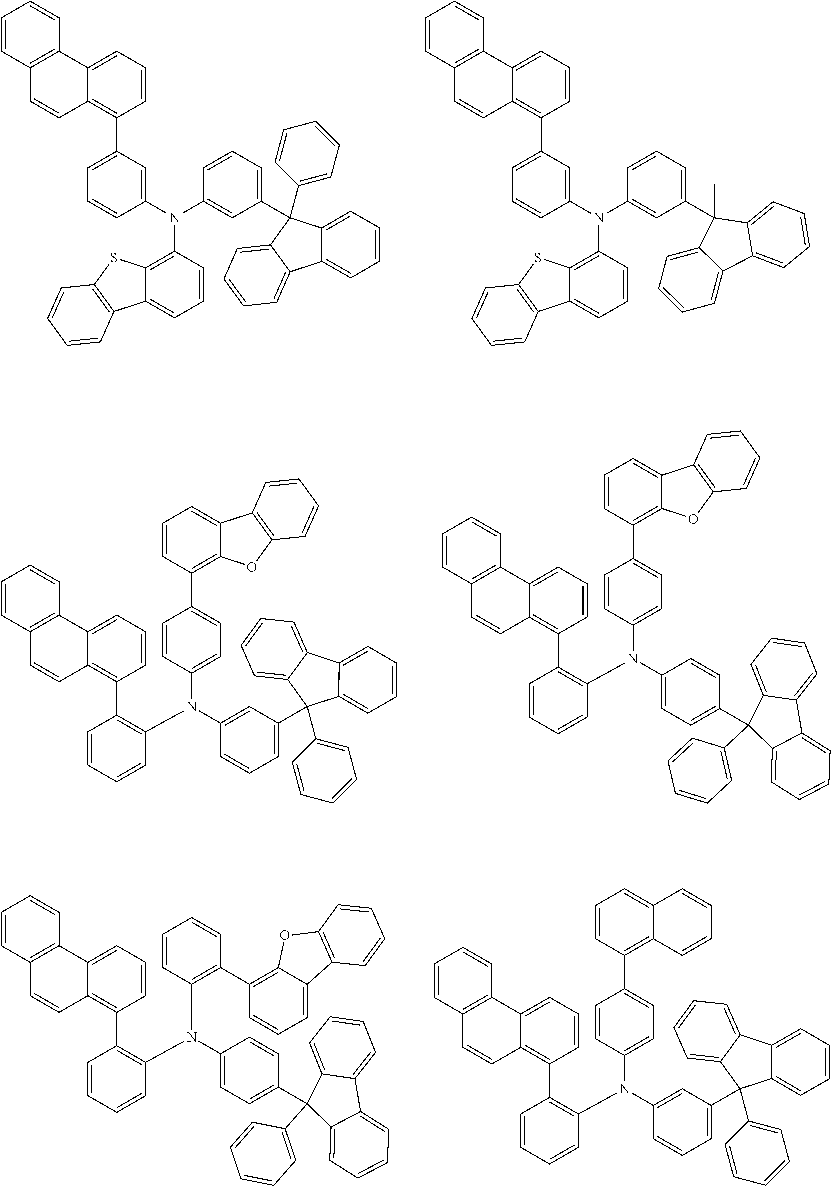

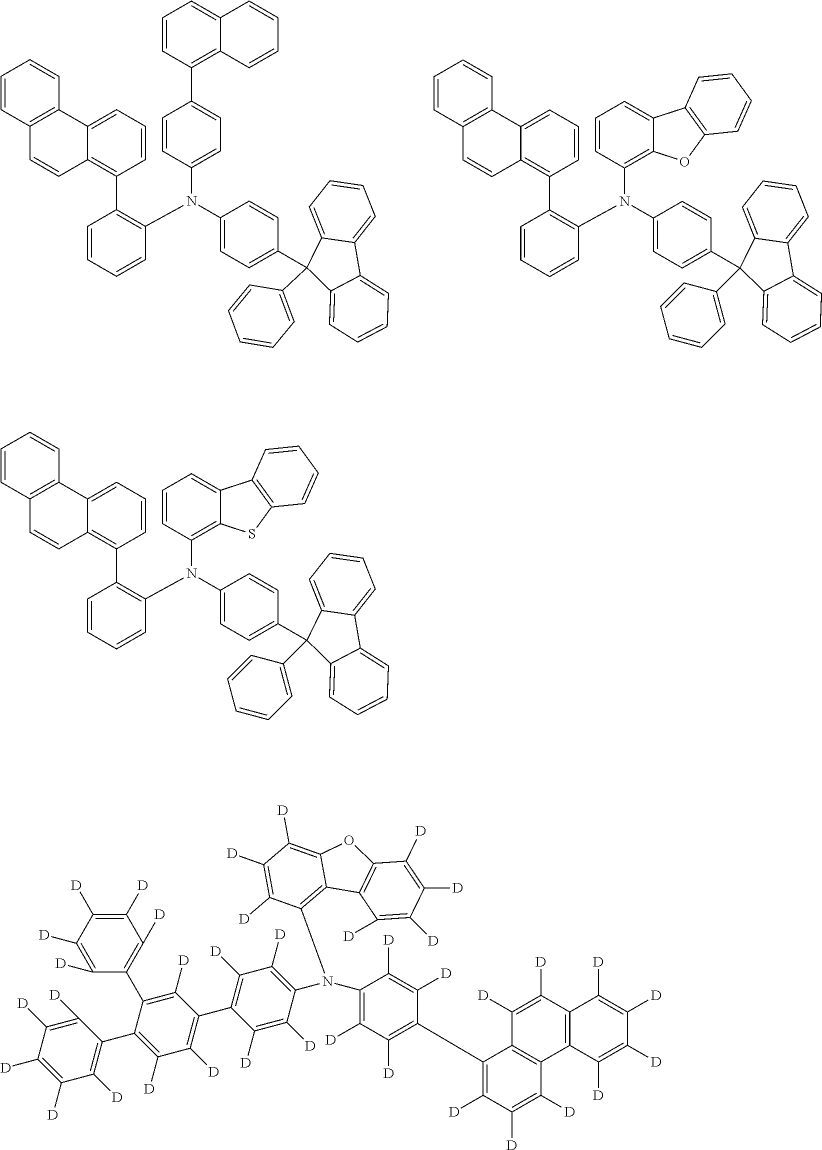

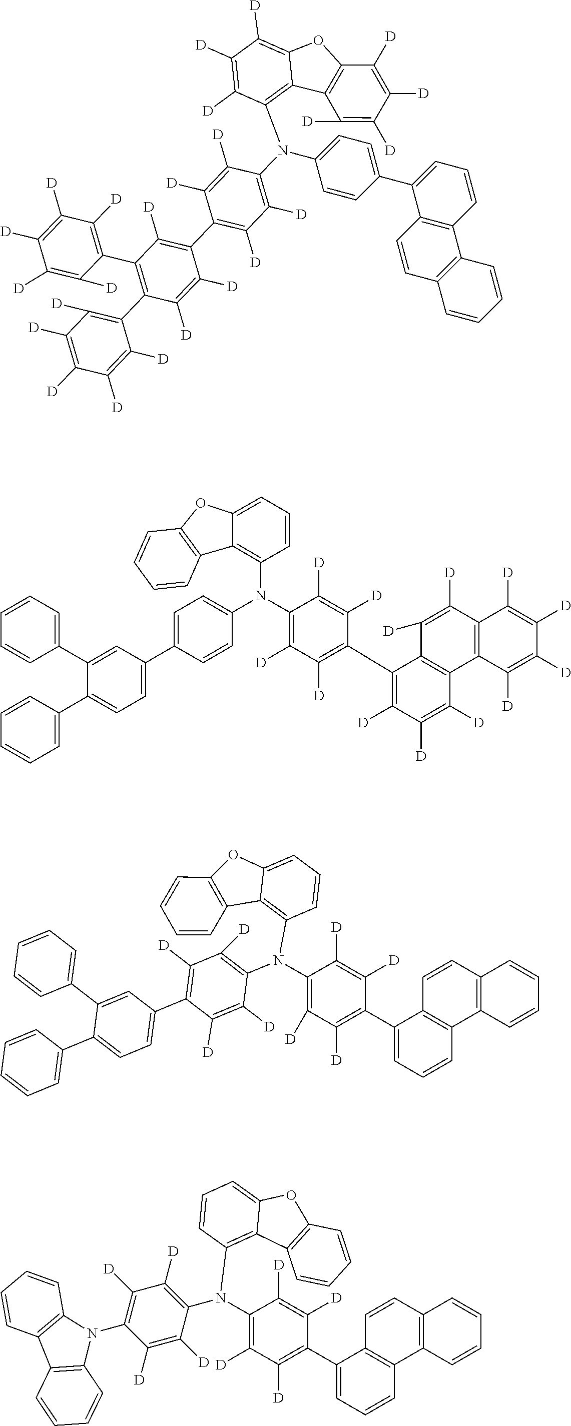

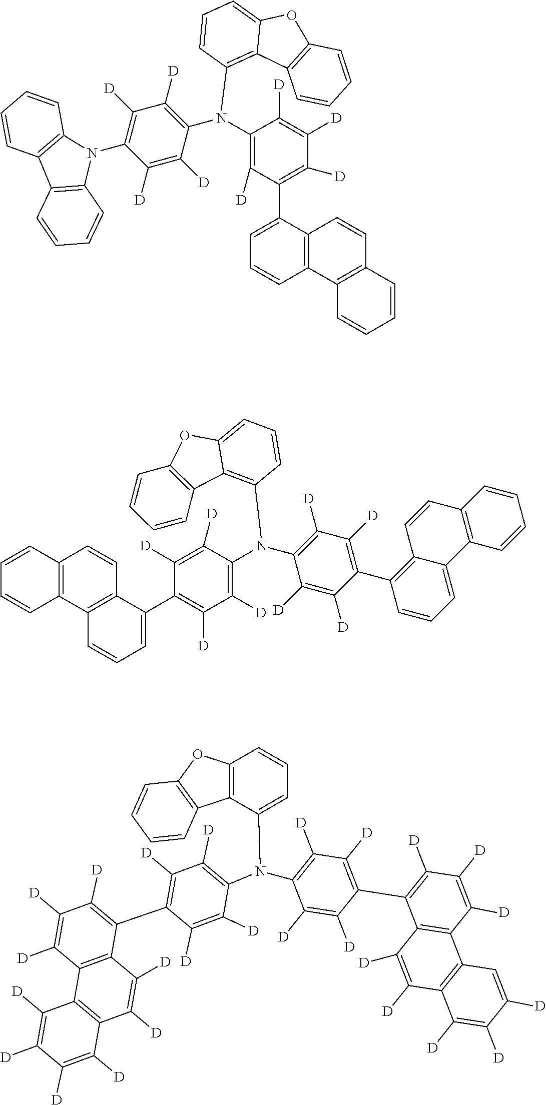 [SMILES: [2H]c1c([2H])c(-c2cccc3c2ccc2ccccc23)c([2H])c(N(c2c([2H])c([2H])c(-n3c4ccccc4c4ccccc43)c([2H])c2[2H])c2cccc3oc4ccccc4c23)c1[2H] ; [2H]c1c([2H])c(N(c2c([2H])c([2H])c(-c3cccc4c3ccc3ccccc34)c([2H])c2[2H])c2cccc3oc4ccccc4c23)c([2H])c([2H])c1-c1cccc2c1ccc1ccccc12 ; [2H]c1cc2c(c([2H])c1[2H])c([2H])c([2H])c1c(-c3c([2H])c([2H])c(N(c4c([2H])c([2H])c(-c5c([2H])c([2H])c([2H])c6c5c([2H])c([2H])c5c([2H])c([2H])c([2H])c([2H])c56)c([2H])c4[2H])c4cccc5oc6ccccc6c45)c([2H])c3[2H])c([2H])c([2H])c([2H])c12]

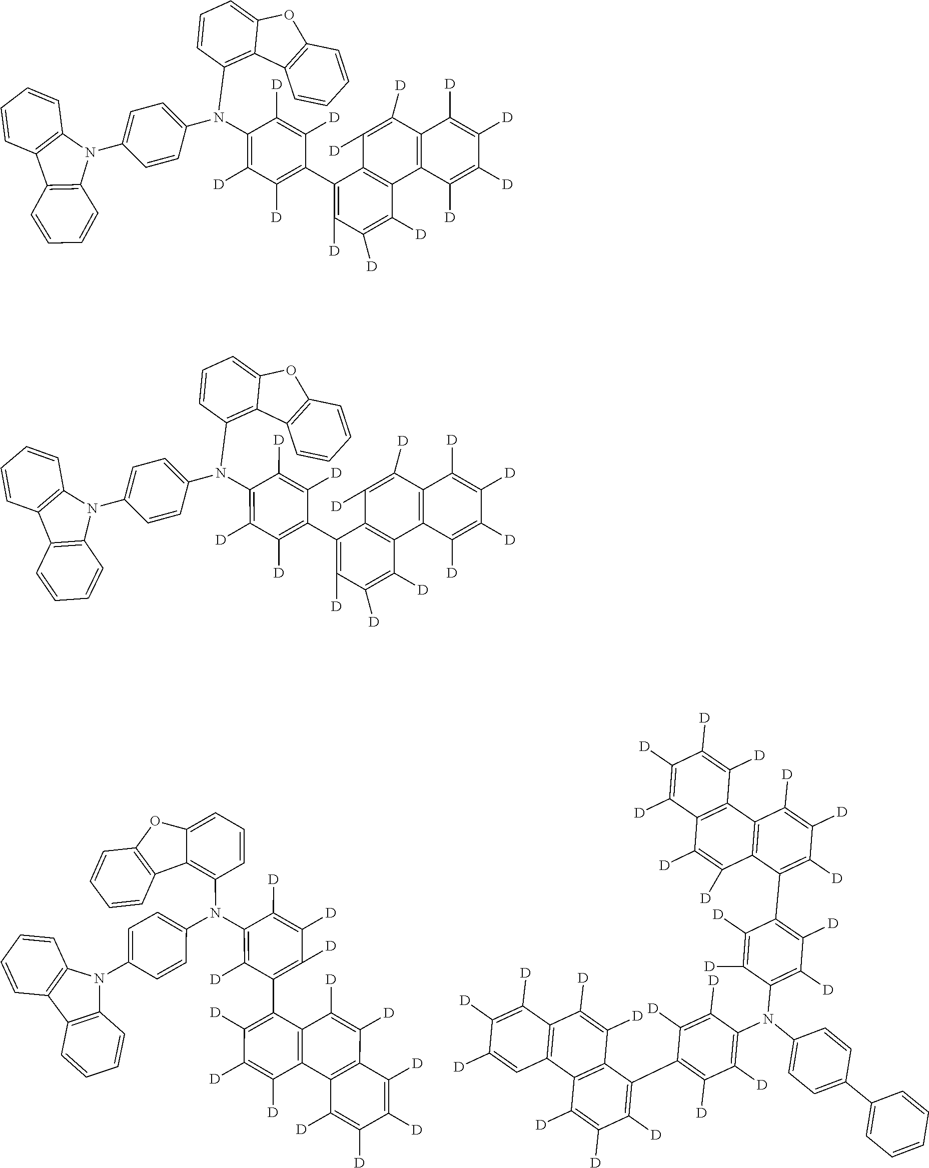

### Material for Organic EL Devices

The material for organic EL devices of the present invention contains the inventive compound. The content of the inventive compound in the material for organic EL devices is 1% by mass or more (including 100%), preferably 10% by mass or more (including 100%), more preferably 50% by mass or more (including 100%), still more preferably 80% by mass or more (including 100%), and particularly preferably 90% by mass or more (including 100%). The material for organic EL devices, which is one embodiment of the present invention, is useful for the production of an organic EL device.

### Organic EL Device

The organic EL device according to one embodiment of the present invention includes an anode, a cathode, and one or more organic layers intervening between the anode and the cathode. The organic layers include a light emitting layer, and at least one layer of the organic layers contains the inventive compound.

Examples of the organic layer containing the inventive compound include a hole transporting zone (such as a hole injecting layer, a hole transporting layer, an electron blocking layer, and an exciton blocking layer) intervening between the anode and the light emitting layer, the light emitting layer, a space layer, and an electron transporting zone (such as an electron injecting layer, an electron transporting layer, and a hole blocking layer) intervening between the cathode and the light emitting layer, but are not limited thereto. The inventive compound is preferably used as a material for the hole transporting zone or the light emitting layer in a fluorescent or phosphorescent EL device, more preferably as a material for the hole transporting zone, still more preferably as a material for the hole injecting layer, the hole transporting layer, the electron blocking layer, or the exciton blocking layer, and particularly preferably as a material for the hole injecting layer or the hole transporting layer.

The organic EL device of the present invention may be a fluorescent or phosphorescent light emission-type monochromatic light emitting device or a fluorescent/phosphorescent hybrid-type white light emitting device, and may be a simple type having a single light emitting unit or a tandem type having a plurality of light emitting units. Above all, the fluorescent light emission-type device is preferred. The “light emitting unit” referred to herein refers to a minimum unit that emits light through recombination of injected holes and electrons, which includes one or more organic layers among which at least one layer is a light emitting layer.

For example, as a representative device configuration of the simple type organic EL device, the following device configuration may be exemplified.

### (1) Anode/Light Emitting Unit/Cathode

The light emitting unit may be a multilayer type having a plurality of phosphorescent light emitting layers or fluorescent light emitting layers. In this case, a space layer may intervene between the light emitting layers for the purpose of preventing excitons generated in the phosphorescent light emitting layer from diffusing into the fluorescent light emitting layer. Representative layer configurations of the simple type light emitting unit are described below. Layers in parentheses are optional.
(a) (hole injecting layer/) hole transporting layer/fluorescent light emitting layer/electron transporting layer (/electron injecting layer)
(b) (hole injecting layer/) hole transporting layer/first fluorescent light emitting layer/second fluorescent light emitting layer/electron transporting layer (/electron injecting layer)
(c) (hole injecting layer/) hole transporting layer/phosphorescent light emitting layer/space layer/fluorescent light emitting layer/electron transporting layer (/electron injecting layer)
(d) (hole injecting layer/) hole transporting layer/first phosphorescent light emitting layer/second phosphorescent light emitting layer/space layer/fluorescent light emitting layer/electron transporting layer (/electron injecting layer)
(e) (hole injecting layer/) hole transporting layer/phosphorescent light emitting layer/space layer/first fluorescent light emitting layer/second fluorescent light emitting layer/electron transporting layer (/electron injecting layer)
(f) (hole injecting layer/) hole transporting layer/electron blocking layer/fluorescent light emitting layer/electron transporting layer (/electron injecting layer)
(g) (hole injecting layer/) hole transporting layer/exciton blocking layer/fluorescent light emitting layer/electron transporting layer (/electron injecting layer)
(h) (hole injecting layer/) first hole transporting layer/second hole transporting layer/fluorescent light emitting layer/electron transporting layer (/electron injecting layer)
(i) (hole injecting layer/) first hole transporting layer/second hole transporting layer/fluorescent light emitting layer/first electron transporting layer/second electron transporting layer (/electron injecting layer)
(j) (hole injecting layer/) hole transporting layer/fluorescent light emitting layer/hole blocking layer/electron transporting layer (/electron injecting layer)
(k) (hole injecting layer/) hole transporting layer/fluorescent light emitting layer/exciton blocking layer/electron transporting layer (/electron injecting layer)
(l) (hole injecting layer/) first hole transporting layer/second hole transporting layer/first fluorescent light emitting layer/second fluorescent light emitting layer/first electron transporting layer/second electron transporting layer (/electron injecting layer)
(m) (hole injecting layer/) first hole transporting layer/second hole transporting layer/third hole transporting layer/first fluorescent light emitting layer/second fluorescent light emitting layer/first electron transporting layer/second electron transporting layer (/electron injecting layer)

The phosphorescent and fluorescent light emitting layers may emit emission colors different from each other, respectively. Specifically, in the light emitting unit (f), a layer configuration, such as (hole injecting layer/) hole transporting layer/first phosphorescent light emitting layer (red light emission)/second phosphorescent light emitting layer (green light emission)/space layer/fluorescent light emitting layer (blue light emission)/electron transporting layer, may be exemplified.

An electron blocking layer may be properly provided between each light emitting layer and the hole transporting layer or the space layer. A hole blocking layer may be properly provided between each light emitting layer and the electron transporting layer. The employment of the electron blocking layer or the hole blocking layer allows to improve the emission efficiency by trapping electrons or holes within the light emitting layer and increasing the probability of charge recombination in the light emitting layer.

As a representative device configuration of the tandem type organic EL device, the following device configuration may be exemplified.

### (2) Anode/First Light Emitting Unit/Intermediate Layer/Second Light Emitting Unit/Cathode

For example, each of the first light emitting unit and the second light emitting unit may be independently selected from the above-described light emitting units.

The intermediate layer is also generally referred to as an intermediate electrode, an intermediate conductive layer, a charge generation layer, an electron withdrawing layer, a connecting layer, or an intermediate insulating layer, and a known material configuration can be used, in which electrons are supplied to the first light emitting unit, and holes are supplied to the second light emitting unit.

FIG. 1 is a schematic view showing an example of the configuration of the organic EL device of the present invention. The organic EL device 1 of this example includes a substrate 2, an anode 3, a cathode 4, and a light emitting unit 10 disposed between the anode 3 and the cathode 4. The light emitting unit 10 includes a light emitting layer 5. A hole transporting zone 6 (such as a hole injecting layer and a hole transporting layer) is provided between the light emitting layer 5 and the anode 3, and an electron transporting zone 7 (such as an electron injecting layer and an electron transporting layer) is provided between the light emitting layer 5 and the cathode 4. In addition, an electron blocking layer (which is not shown in the figure) may be provided on the side of the anode 3 of the light emitting layer 5, and a hole blocking layer (which is not shown in the figure) may be provided on the side of the cathode 4 of the light emitting layer 5. According to the configuration, electrons and holes are trapped in the light emitting layer 5, thereby enabling one to further increase the production efficiency of excitons in the light emitting layer 5.

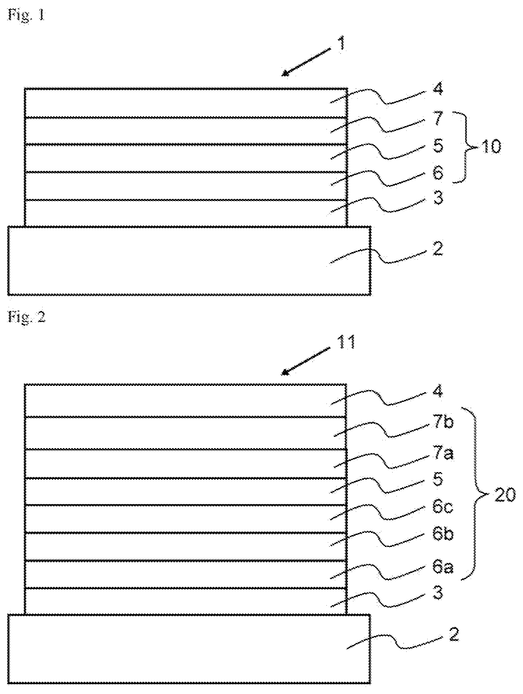

FIG. 2 is a schematic view showing another configuration of the organic EL device of the present invention. The organic EL device 11 of this example includes a substrate 2, an anode 3, a cathode 4, and a light emitting unit 20 disposed between the anode 3 and the cathode 4. The light emitting unit 20 includes a first light emitting layer 5a and a second light emitting layer 5b. A hole transporting zone disposed between the anode 3 and the first light emitting layer 5a is formed of a hole injecting layer 6a, a first hole transporting layer 6b, and a second hole transporting layer 6c. In addition, the electron transporting zone disposed between the second light emitting layer 5b and the cathode 4 is formed of a first electron transporting layer 7a and a second electron transporting layer 7b.

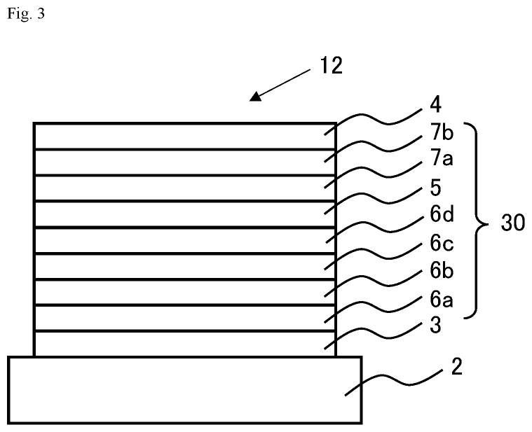

FIG. 3 is a schematic view showing another configuration of the organic EL device of the present invention. The organic EL device 12 of this example includes a substrate 2, an anode 3, a cathode 4, and a light emitting unit 30 disposed between the anode 3 and the cathode 4. The light emitting unit 30 includes a first light emitting layer 5a and a second light emitting layer 5b. A hole transporting zone disposed between the anode 3 and the first light emitting layer 5a is formed of a hole injecting layer 6a, a first hole transporting layer 6b, a second hole transporting layer 6c, and a third hole transporting layer 6d. In addition, the electron transporting zone disposed between the second light emitting layer 5b and the cathode 4 is formed of a first electron transporting layer 7a and a second electron transporting layer 7b.

In the present invention, a host combined with a fluorescent dopant material (a fluorescent emitting material) is referred to as a fluorescent host, and a host combined with a phosphorescent dopant material is referred to as a phosphorescent host. The fluorescent host and the phosphorescent host are not distinguished from each other merely by the molecular structures thereof. Specifically, the phosphorescent host means a material that forms a phosphorescent light emitting layer containing a phosphorescent dopant, but does not mean unavailability as a material that forms a fluorescent light emitting layer. The same also applies to the fluorescent host.

### Substrate

The substrate is used as a support of the organic EL device. Examples of the substrate include a plate of glass, quartz, and plastic. In addition, a flexible substrate may be used. Examples of the flexible substrate include a plastic substrate made of polycarbonate, polyarylate, polyether sulfone, polypropylene, polyester, polyvinyl fluoride, or polyvinyl chloride. In addition, an inorganic vapor deposition film can be used.

### Anode

It is preferred that a metal, an alloy, an electrically conductive compound, or a mixture thereof which has a high work function (specifically 4.0 eV or more) is used for the anode formed on the substrate. Specific examples thereof include indium oxide-tin oxide (ITO: Indium Tin Oxide), indium oxide-tin oxide containing silicon or silicon oxide, indium oxide-zinc oxide, indium oxide containing tungsten oxide and zinc oxide, and graphene. Besides, examples thereof include gold (Au), platinum (Pt), nickel (Ni), tungsten (W), chromium (Cr), molybdenum (Mo), iron (Fe), cobalt (Co), copper (Cu), palladium (Pd), titanium (Ti), or nitrides of the metals (for example, titanium nitride).

These materials are usually deposited by a sputtering method. For example, through a sputtering method, it is possible to form indium oxide-zinc oxide by using a target in which 1 to 10 wt % of zinc oxide is added to indium oxide, and to form indium oxide containing tungsten oxide and zinc oxide by using a target containing 0.5 to 5 wt % of tungsten oxide and 0.1 to 1 wt % of zinc oxide with respect to indium oxide. Besides, the manufacturing may be performed by a vacuum vapor deposition method, a coating method, an inkjet method, a spin coating method, or the like.

### Hole Transporting Zone

As described above, the organic layer may include a hole transporting zone between the anode and the light emitting layer. The hole transporting zone is composed of a hole injecting layer, a hole transporting layer, an electron blocking layer, and the like. It is preferred that the hole transporting zone contains the inventive compound. It is preferred that at least one of these layers constituting the hole transporting layer contains the inventive compound, and it is particularly more preferred that the hole transporting layer contains the inventive compound.

The hole injecting layer formed in contact with the anode is formed by using a material that facilitates hole injection regardless of a work function of the anode, and thus, it is possible to use materials generally used as an electrode material (for example, metals, alloys, electrically conductive compounds, or mixtures thereof, elements belonging to Group 1 or Group 2 of the periodic table of the elements).

It is also possible to use elements belonging to Group 1 or Group 2 of the periodic table of the elements, which are materials having low work functions, that is, alkali metals, such as lithium (Li) and cesium (Cs), alkaline earth metals, such as magnesium (Mg), calcium (Ca), and strontium (Sr), and alloys containing these (such as MgAg and AlLi), and rare earth metals, such as europium (Eu), and ytterbium (Yb) and alloys containing these. When the anode is formed by using the alkali metals, the alkaline earth metals, and alloys containing these, a vacuum vapor deposition method or a sputtering method can be used. Further, when a silver paste or the like is used, a coating method, an inkjet method, or the like can be used.

### Hole Injecting Layer

The hole injecting layer is a layer containing a material having a high hole injection capability (a hole injecting material) and is provided between the anode and the light emitting layer, or between the hole transporting layer, if exists, and the anode.

As the hole injecting material except the inventive compound, molybdenum oxide, titanium oxide, vanadium oxide, rhenium oxide, ruthenium oxide, chromium oxide, zirconium oxide, hafnium oxide, tantalum oxide, silver oxide, tungsten oxide, and manganese oxide can be used.

Examples of the hole injecting layer material also include aromatic amine compounds as low-molecular weight organic compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1).

High-molecular weight compounds (such as oligomers, dendrimers, and polymers) may also be used. Examples thereof include high-molecular weight compounds, such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide] (abbreviation: PTPDMA), and poly[N,N′-bis(4-butylphenyl)-N,N′-bis(phenyl)benzidine] (abbreviation: Poly-TPD). In addition, high-molecular weight compounds to which an acid is added, such as poly(3,4-ethylenedioxythiophene)/poly(styrene sulfonic acid) (PEDOT/PSS), and polyaniline/poly(styrenesulfonic acid) (PAni/PSS), can also be used.

Furthermore, it is also preferred to use an acceptor material, such as a hexaazatriphenylene (HAT) compound represented by the following formula (K).

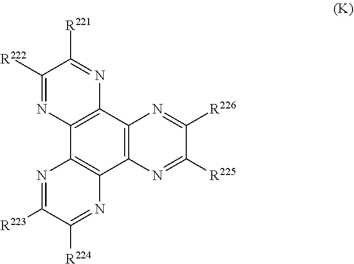

In the aforementioned formula, R²²¹ to R²²⁶ each independently represent a cyano group, —CONH₂, a carboxy group, or —COOR²²⁷ (R²²⁷ represents an alkyl group having 1 to 20 carbon atoms or a cycloalkyl group having 3 to 20 carbon atoms). In addition, adjacent two selected from R²²¹ and R²²², R²²³ and R²²⁴, and R²²⁵ and R²²⁶ may be bonded to each other to form a group represented by —CO—O—CO—.

Examples of R²²⁷ include a methyl group, an ethyl group, a n-propyl group, an isopropyl group, a n-butyl group, an isobutyl group, a t-butyl group, a cyclopentyl group, and a cyclohexyl group.

### Hole Transporting Layer

The hole transporting layer is a layer containing a material having a high hole transporting capability (a hole transporting material) and is provided between the anode and the light emitting layer, or between the hole injecting layer, if exists, and the light emitting layer. The inventive compound can be used as the hole transporting layer either singly or as combined with the compound mentioned below.

The hole transporting layer may have a single layer structure or a multilayer structure including two or more layers. For example, the hole transporting layer may have a two layer structure including a first hole transporting layer (anode side) and a second hole transporting layer (cathode side). That is, the hole transporting zone may include the first hole transporting layer on the anode side and the second hole transporting layer on the cathode side. In addition, the hole transporting layer may have a three layer structure including a first hole transporting layer, a second hole transporting layer, and a third hole transporting layer in this order from the anode side. That is, the third hole transporting layer may be disposed between the second hole transporting layer and the light emitting layer.

In one embodiment of the present invention, the hole transporting layer having a single layer structure is preferably disposed adjacent to the light emitting layer, and the hole transporting layer that is closest to the cathode in the multilayer structure, such as the second hole transporting layer of the two-layer structure or the third hole transporting layer of the three-layer structure, is preferably disposed adjacent to the light emitting layer. In another embodiment of the present invention, an electron blocking layer described later or the like may be interposed between the hole transporting layer having a single layer structure and the light emitting layer, or between the hole transporting layer that is closest to the light emitting layer in the multilayer structure and the light emitting layer.

In the case where the hole transporting layer has a two layer structure, at least one of the first hole transporting layer and the second hole transporting layer contains the inventive compound. In other words, the inventive compound may be contained in one or both of the first hole transporting layer and the second hole transporting layer. In one embodiment of the present invention, the inventive compound is preferably contained in the second hole transporting layer. That is, the inventive compound is preferably contained only in the second hole transporting layer, or the inventive compound is preferably contained in the first hole transporting layer and the second hole transporting layer.

In the case where the hole transporting layer has a three layer structure, at least one of the first to third hole transporting layers contains the inventive compound. That is, the inventive compound may be contained in only one of the first to third hole transporting layers, may be contained in any two of the first to third hole transporting layers, or may be contained in all of the first to third hole transporting layers. In one embodiment of the present invention, the inventive compound is preferably contained in the third hole transporting layer. That is, it is preferred that the inventive compound is contained only in the third hole transporting layer or that the inventive compound is contained in the third hole transporting layer and one or both of the first hole transporting layer and the second hole transporting layer.

In one embodiment of the present invention, the inventive compound contained in each of the transporting layers is preferably a protium compound from the viewpoint of production cost. The protium compound is an inventive compound in which all hydrogen atoms in the inventive compound are protium atoms.

Therefore, the present invention includes an organic EL device containing an inventive compound in which one or both of the first hole transporting layer and the second hole transporting layer (in the case of a two layer structure) and at least one of the first to third hole transporting layers is substantially composed of only a protium compound. The expression “the inventive compound substantially composed of only a protium compound” means that the content ratio of the protium compound to the total amount of the inventive compound is 90 mol % or more, preferably 95 mol % or more, and more preferably 99 mol % or more (each including 100%).

As the hole transporting material except the inventive compound, for example, an aromatic amine compound, a carbazole derivative, and an anthracene derivative can be used.

Examples of the aromatic amine compound include 4,4′-bis[N-(1-naphthyl)-N-phenylamino]biphenyl (abbreviation: NPB) or N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluoren-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: BSPB). The aforementioned compounds have a hole mobility of 10⁻⁶ cm²/Vs or more.

Examples of the carbazole derivative include 4,4′-di(9-carbazolyl)biphenyl (abbreviation: CBP), 9-[4-(9-carbazolyl)phenyl]-10-phenylanthracene (abbreviation: CzPA), and 9-phenyl-3-[4-(10-phenyl-9-anthryl)phenyl]-9H-carbazole (abbreviation: PCzPA).

Examples of the anthracene derivative include 2-t-butyl-9,10-di(2-naphthyl)anthracene (abbreviation: t-BuDNA), 9,10-di(2-naphthyl)anthracene (abbreviation: DNA), and 9,10-diphenylanthracene (abbreviation: DPAnth).

High-molecular weight compounds, such as poly(N-vinylcarbazole) (abbreviation: PVK) and poly(4-vinyltriphenylamine) (abbreviation: PVTPA), can also be used.

However, compounds other than those as mentioned above can also be used so long as they are compounds high in the hole transporting capability rather than in the electron transporting capability.

In one embodiment of the organic EL device having a two layer structure or two or more hole transport layers according to the present invention, the first hole transporting layer preferably contains a compound represented by the following formula (11) or formula (12).

In the organic EL device having a hole transporting layer having a three layer structure of the present invention, it is preferred that one or both of the first hole transporting layer and the second hole transporting layer contains one or a plurality of compounds represented by the following formula (11) or formula (12).

In the organic EL device having a hole transporting layer having an n-layer structure (n is an integer of 4 or more) of the present invention, it is preferred that at least one of the first to (n−1)th hole transporting layers contains one or a plurality of compounds represented by the following formula (11) or formula (12).

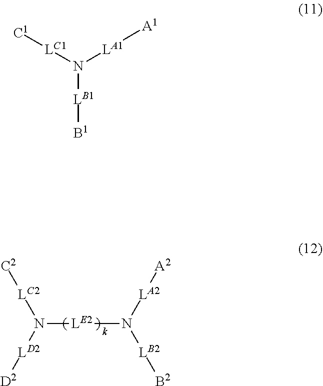

In the formula (11) and the formula (12),
L^{A1}, L^{B1}, L^{C1}, L^{A2}, L^{B2}, L^{C2}, and L^{D2} are each independently a single bond, a substituted or unsubstituted arylene group having 6 to 50 ring carbon atoms, or a substituted or unsubstituted divalent heterocyclic group having 5 to 50 ring atoms;
k is 1, 2, 3, or 4;
in the case where k is 1, L^{E2} is a substituted or unsubstituted arylene group having 6 to 50 ring carbon atoms, or a substituted or unsubstituted divalent heterocyclic group having 5 to 50 ring atoms,
in the case where k is 2, 3, or 4, two, three or four L^{E2}'s are the same as or different from each other,
in the case where k is 2, 3, or 4, a plurality of L^{E2}'s are bonded to each other to form a substituted or unsubstituted monocyclic ring, are bonded to each other to form a substituted or unsubstituted fused ring, or are not bonded to each other, and
L^{E2}, which does not form a monocyclic ring and does not form a fused ring, is a substituted or unsubstituted arylene group having 6 to 50 ring carbon atoms, or a substituted or unsubstituted divalent heterocyclic group having 5 to 50 ring atoms;
A¹, B¹, C¹, A², B², C², and D² are each independently a substituted or unsubstituted aryl group having 6 to 50 ring carbon atoms, a substituted or unsubstituted heterocyclic group having 5 to 50 ring atoms, or —Si(R′₉₀₁)(R′₉₀₂)(R′₉₀₃);
R′₉₀₁, R′₉₀₂, and R′₉₀₃ are each independently a substituted or unsubstituted aryl group having 6 to 50 ring carbon atoms;
in the case where a plurality of R′₉₀₁'s exist, the plurality of R′₉₀₁'s are the same as or different from each other;
in the case where a plurality of R′₉₀₂'s exist, the plurality of R′₉₀₂'s are the same as or different from each other;
in the case where a plurality of R′₉₀₃'s exist, the plurality of R′₉₀₃'s are the same as or different from each other.
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
in the case where a plurality of R₉₀₁'s are present, the plurality of R₉₀₁'s are the same as or different from each other,
in the case where a plurality of R₉₀₂'s are present, the plurality of R₉₀₂'s are the same as or different from each other,
in the case where a plurality of R₉₀₃'s are present, the plurality of R₉₀₃'s are the same as or different from each other,
in the case where a plurality of R₉₀₄'s are present, the plurality of R₉₀₄'s are the same as or different from each other,
in the case where a plurality of R₉₀₅'s are present, the plurality of R₉₀₅'s are the same as or different from each other,
in the case where a plurality of R₉₀₆'s are present, the plurality of R₉₀₆'s are the same as or different from each other, and
in the case where a plurality of R₉₀₇'s are present, the plurality of R₉₀₇'s are the same as or different from each other.

In the formula (11) and the formula (12), A¹, B¹, C¹, A², B², C², and D² are preferably each independently selected from a substituted or unsubstituted phenylene group, a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, a substituted or unsubstituted dibenzothiophenyl group, and a substituted or unsubstituted carbazolyl group.

More preferably, at least one of A¹, B¹, and C¹ in the formula (11) and at least one of A², B², C², and D² in the formula (12) are a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, a substituted or unsubstituted dibenzothiophenyl group, or a substituted or unsubstituted carbazolyl group.

The fluorenyl group that can be taken by A¹, B¹, C¹, A², B², C², and D² may have a substituent at the 9-position, and may be, for example, a 9,9-dimethylfluorenyl group or a 9,9-diphenylfluorenyl group. Further, substituents at the 9-positions may form a ring, for example, a fluorene skeleton or a xanthene skeleton may be formed between substituents at the 9-positions.

L^{A1}, L^{B1}, L^{C1}, L^{A2}, L^{B2}, L^{C2}, and L^{D2} are preferably each independently a single bond or a substituted or unsubstituted arylene group having 6 to 12 ring carbon atoms.

Specific examples of the compounds represented by the formula (11) and the formula (12) include the following compounds.

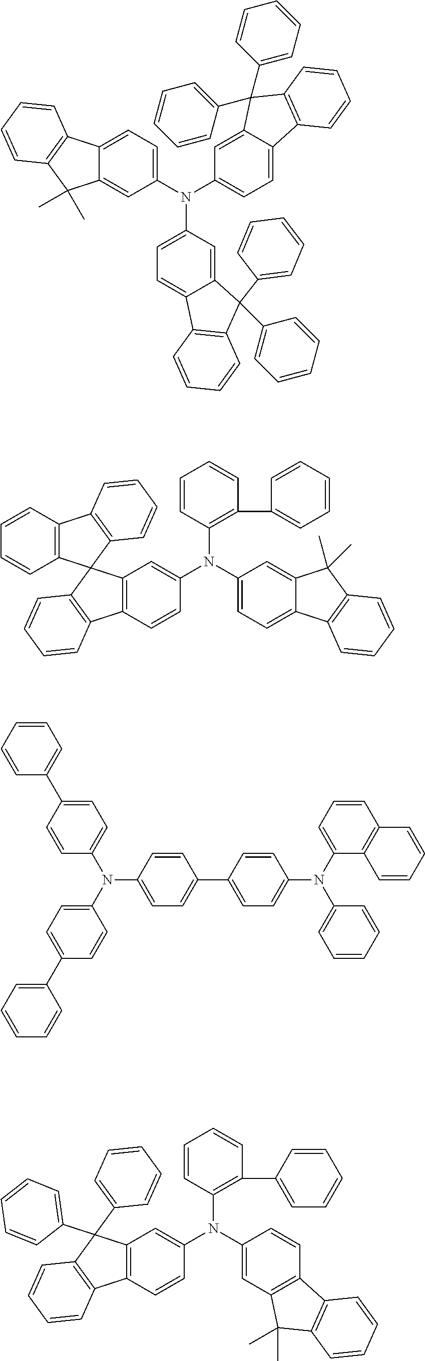 [SMILES: CC1(C)c2ccccc2-c2ccc(N(c3ccc4c(c3)C(c3ccccc3)(c3ccccc3)c3ccccc3-4)c3ccc4c(c3)C(c3ccccc3)(c3ccccc3)c3ccccc3-4)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3ccc(-c4ccccc4)cc3)c3ccc(-c4ccc(N(c5ccccc5)c5cccc6ccccc56)cc4)cc3)cc2)cc1 ; CC1(C)c2ccccc2-c2ccc(N(c3ccc4c(c3)C(c3ccccc3)(c3ccccc3)c3ccccc3-4)c3ccccc3-c3ccccc3)cc21]

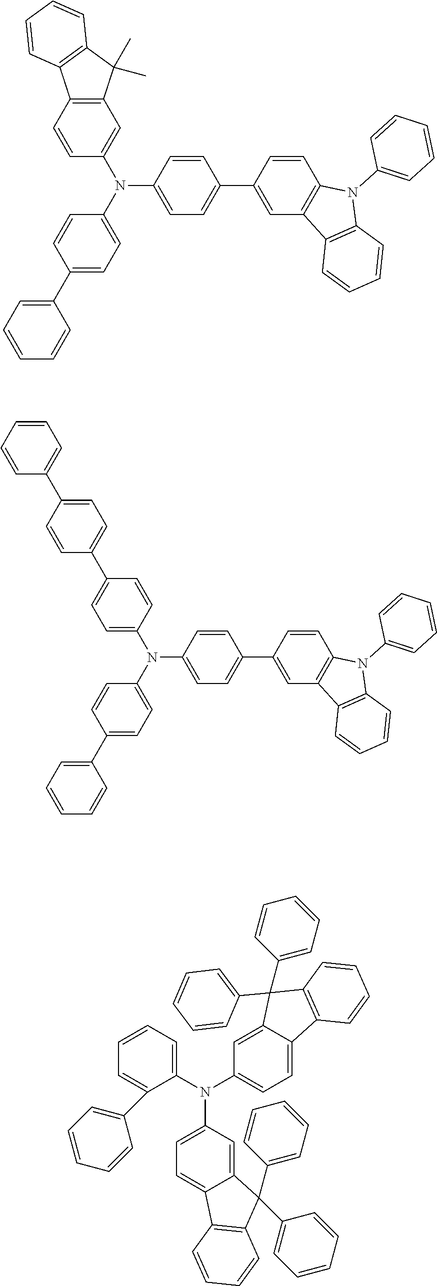 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

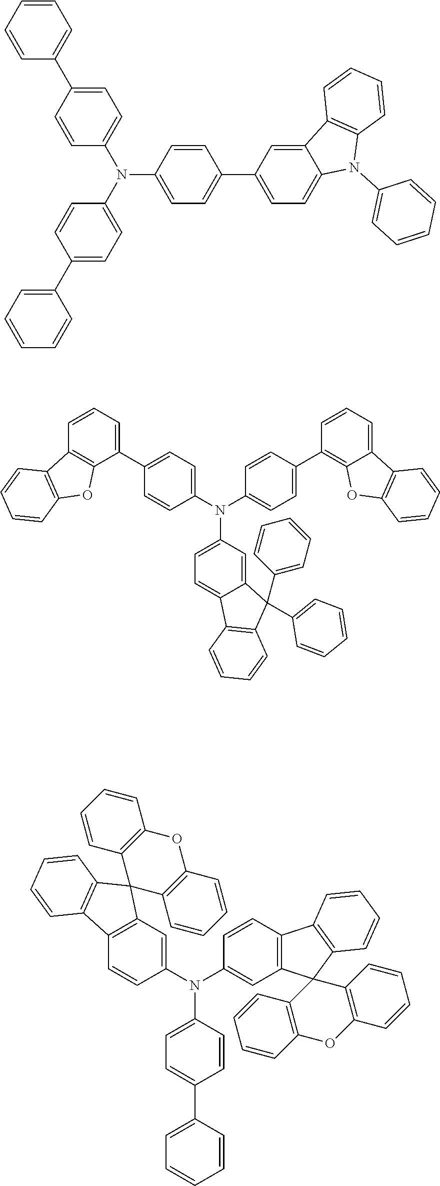 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1 ; c1ccc(-c2ccc(N(c3ccc4c(c3)C3(c5ccccc5Oc5ccccc53)c3ccccc3-4)c3ccc4c(c3)C3(c5ccccc5Oc5ccccc53)c3ccccc3-4)cc2)cc1]

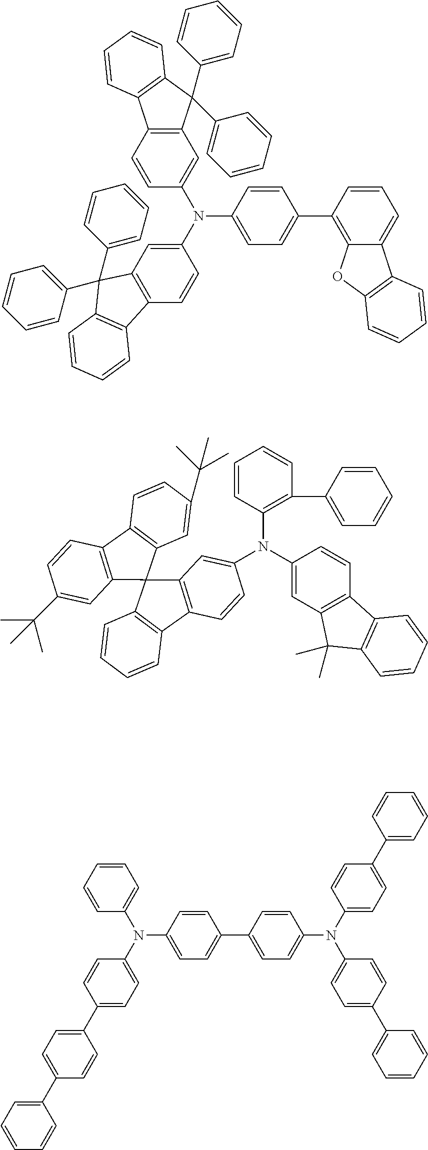 [SMILES: c1ccc(C2(c3ccccc3)c3ccccc3-c3ccc(N(c4ccc(-c5cccc6c5oc5ccccc56)cc4)c4ccc5c(c4)C(c4ccccc4)(c4ccccc4)c4ccccc4-5)cc32)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

### Dopant Material of Light Emitting Layer

The light emitting layer is a layer containing a material having a high light emitting property (a dopant material), and various materials can be used. For example, a fluorescent emitting material or a phosphorescent emitting material can be used as the dopant material. The fluorescent emitting material is a compound that emits light from a singlet excited state, and the phosphorescent emitting material is a compound that emits light from a triplet excited state.

In one embodiment of the organic EL device according to the present invention, the light emitting layer is a single layer.

In another embodiment of the organic EL device according to the present invention, the light emitting layer includes a first light emitting layer and a second light emitting layer.

Examples of a blue-based fluorescent emitting material that can be used for the light emitting layer include a pyrene derivative, a styrylamine derivative, a chrysene derivative, a fluoranthene derivative, a fluorene derivative, a diamine derivative, and a triarylamine derivative. Specific examples thereof include N,N′-bis[4-(9H-carbazole-9-yl)phenyl]-N,N′-diphenylstilbene-4,4′-diamine (abbreviation: YGA2S), 4-(9H-carbazole-9-yl)-4′-(10-phenyl-9-anthryl)triphenylamine (abbreviation: YGAPA), and 4-(10-phenyl-9-anthryl)-4′-(9-phenyl-9H-carbazole-3-yl)triphenylamine (abbreviation: PCBAPA).

Examples of a green-based fluorescent emitting material that can be used for the light emitting layer include an aromatic amine derivative. Specific examples thereof include N-(9,10-diphenyl-2-anthryl)-N,9-diphenyl-9H-carbazole-3-amine (abbreviation: 2PCAPA), N-[9,10-bis(1,1′-biphenyl-2-yl)-2-anthryl]-N,9-diphenyl-9H-carbazole-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazole-9-yl)phenyl]-N-phenylanthracene-2-amine (abbreviation: 2YGABPhA), and N,N,9-triphenylanthracene-9-amine (abbreviation: DPhAPhA).

Examples of a red-based fluorescent emitting material that can be used for the light emitting layer include a tetracene derivative and a diamine derivative. Specific examples thereof include N,N,N′,N′-tetrakis(4-methylphenyl)tetracene-5,11-diamine (abbreviation: p-mPhTD) and 7,14-diphenyl-N,N,N′,N′-tetrakis(4-methylphenyl)acenaphtho[1,2-a]fluoranthene-3,10-diamine (abbreviation: p-mPhAFD).

In one embodiment of the present invention, the light emitting layer preferably contains a fluorescent light emitting material (fluorescent dopant material).

Examples of a blue-based phosphorescent emitting material that can be used for the light emitting layer include a metal complex, such as an iridium complex, an osmium complex, and a platinum complex. Specific examples thereof include bis[2-(4′,6′-difluorophenyl)pyridinato-N,C2′]iridium(III)tetrakis(1-pyrazolyl)borate (abbreviation: FIr6), bis[2-(4′,6′-difluorophenyl)pyridinato-N,C2′]iridium(III)picolinate (abbreviation: FIrpic), bis[2-(3′,5′-bistrifluoromethylphenyl)pyridinato-N,C2′]iridium(III)picolinate (abbreviation: Ir(CF3ppy)2(pic)), and bis[2-(4′,6′-difluorophenyl)pyridinato-N,C2′]iridium(III)acetylacetonate (abbreviation: FIracac).

Examples of a green-based phosphorescent emitting material that can be used for the light emitting layer include an iridium complex. Examples thereof include tris(2-phenylpyridinato-N,C2′)iridium(III) (abbreviation: Ir(ppy)3), bis(2-phenylpyridinato-N,C2′)iridium(III)acetylacetonate (abbreviation: Ir(ppy)2(acac)), bis(1,2-diphenyl-1H-benzimidazolato)iridium(III)acetylacetonate (abbreviation: Ir(pbi)2(acac)), and bis(benzo[h]quinolinato)iridium(III)acetylacetonate (abbreviation: Ir(bzq)2(acac)).

Examples of a red-based phosphorescent emitting material that can be used for the light emitting layer include a metal complex, such as an iridium complex, a platinum complex, a terbium complex, and a europium complex. Specific examples thereof include organic metal complexes, such as bis[2-(2′-benzo[4,5-a]thienyl)pyridinato-N,C3′]iridium(III)acetylacetonate (abbreviation: Ir(btp)2(acac)), bis(1-phenylisoquinolinato-N,C2′)iridium(III)acetylacetonate (abbreviation: Ir(piq)2(acac)), (acetylacetonate)bis[2,3-bis(4-fluorophenyl)quinoxalinato]iridium(III) (abbreviation: Ir(Fdpq)2(acac)), and 2,3,7,8,12,13,17,18-octaethyl-21H,23H-porphyrinplatinum(II) (abbreviation: PtOEP).

Rare earth metal complexes, such as tris(acetylacetonate) (monophenanthroline)terbium(III) (abbreviation: Tb(acac)3(Phen)), tris(1,3-diphenyl-1,3-propanedionate)(monophenanthroline)europium(III) (abbreviation: Eu(DBM)3(Phen)), and tris[1-(2-thenoyl)-3,3,3-trifluoroacetonate](monophenanthroline)europium(III) (abbreviation: Eu(TTA)3(Phen)), emit light from rare earth metal ions (electron transition between different multiplicities), and thus may be used as the phosphorescent emitting material.

### Host Material of Light Emitting Layer

The light emitting layer may have a configuration in which the aforementioned dopant material is dispersed in another material (a host material). The host material is preferably a material that has a higher lowest unoccupied orbital level (LUMO level) and a lower highest occupied orbital level (HOMO level) than the dopant material.

Examples of the host material include: (1) a metal complex, such as an aluminum complex, a beryllium complex, and a zinc complex, (2) a heterocyclic compound, such as an oxadiazole derivative, a benzimidazole derivative, and a phenanthroline derivative, (3) a fused aromatic compound, such as a carbazole derivative, an anthracene derivative, a phenanthrene derivative, a pyrene derivative, and a chrysene derivative, or (4) an aromatic amine compound, such as a triarylamine derivative and a fused polycyclic aromatic amine derivative.

For example, metal complexes, such as tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ);
heterocyclic compounds, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), bathophenanthroline (abbreviation: BPhen), and bathocuproine (abbreviation: BCP);
fused aromatic compounds, such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl(abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), and 6,12-dimethoxy-5,11-diphenylchrysene; and
aromatic amine compounds, such as N,N-diphenyl-9-[4-(10-phenyl-9-anthryl)phenyl]-9H-carbazole-3-amine (abbreviation: CzATPA), 4-(10-phenyl-9-anthryl)triphenylamine (abbreviation: DPhPA), N,9-diphenyl-N-[4-(10-phenyl-9-anthryl)phenyl]-9H-carbazole-3-amine (abbreviation: PCAPA), N,9-diphenyl-N-{4-[4-(10-phenyl-9-anthryl)phenyl]phenyl}-9H-carbazole-3-amine (abbreviation: PCAPBA), N-(9,10-diphenyl-2-anthryl)-N,9-diphenyl-9H-carbazole-3-amine (abbreviation: 2PCAPA), 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB) can be used. A plurality of host materials may be used.

In particular, in the case of a blue fluorescent device, it is preferred to use the following anthracene compounds as the host material.

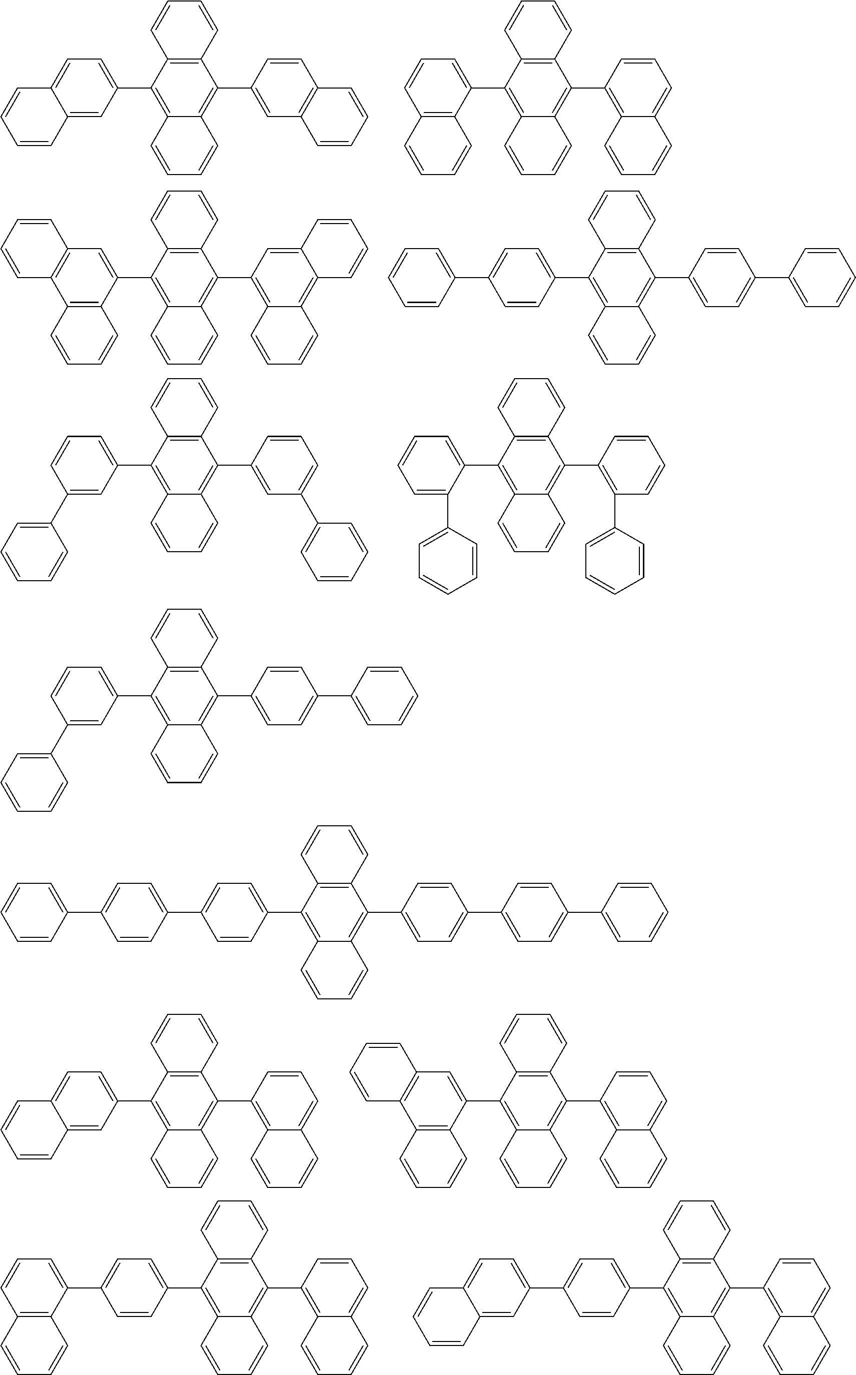

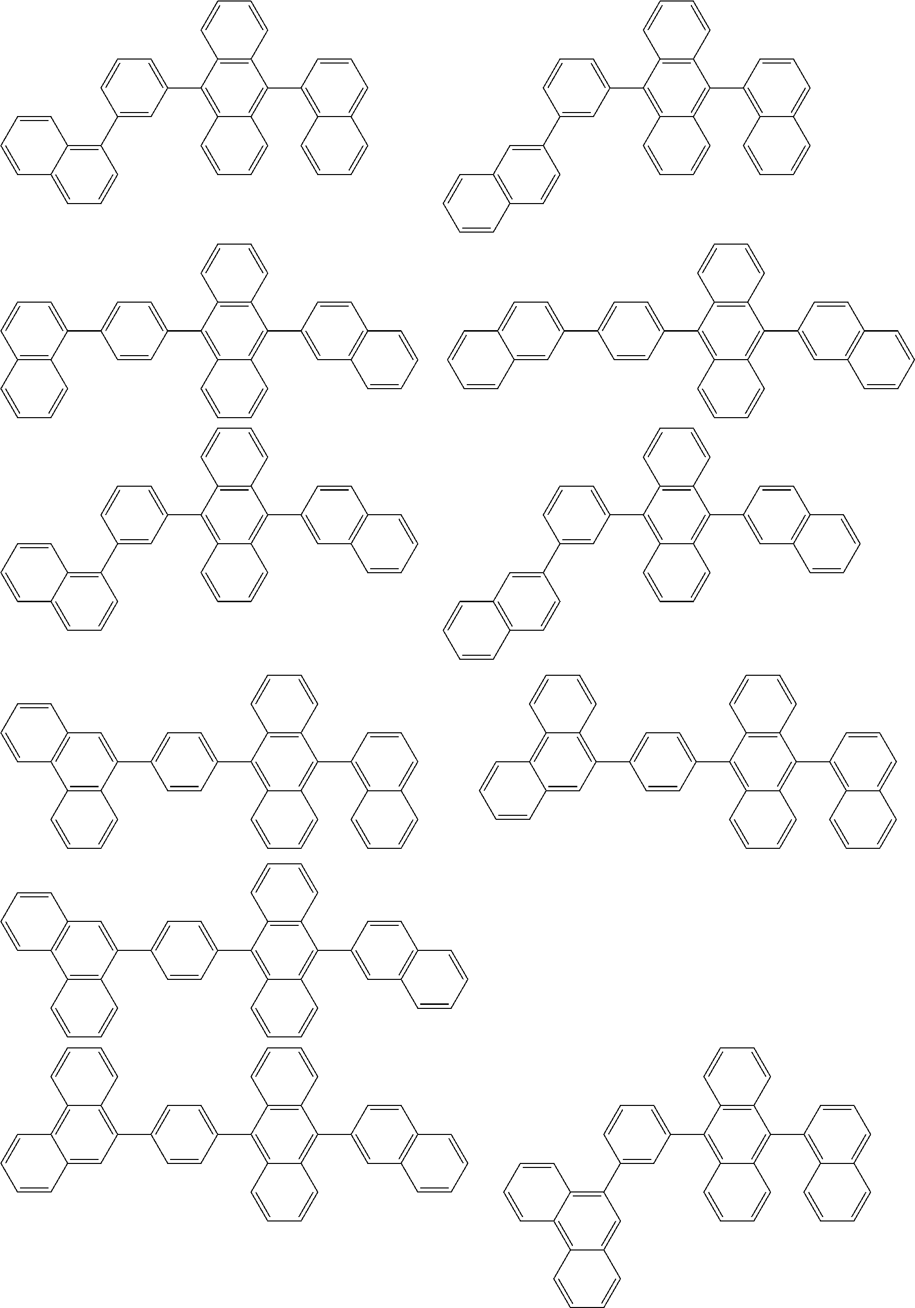

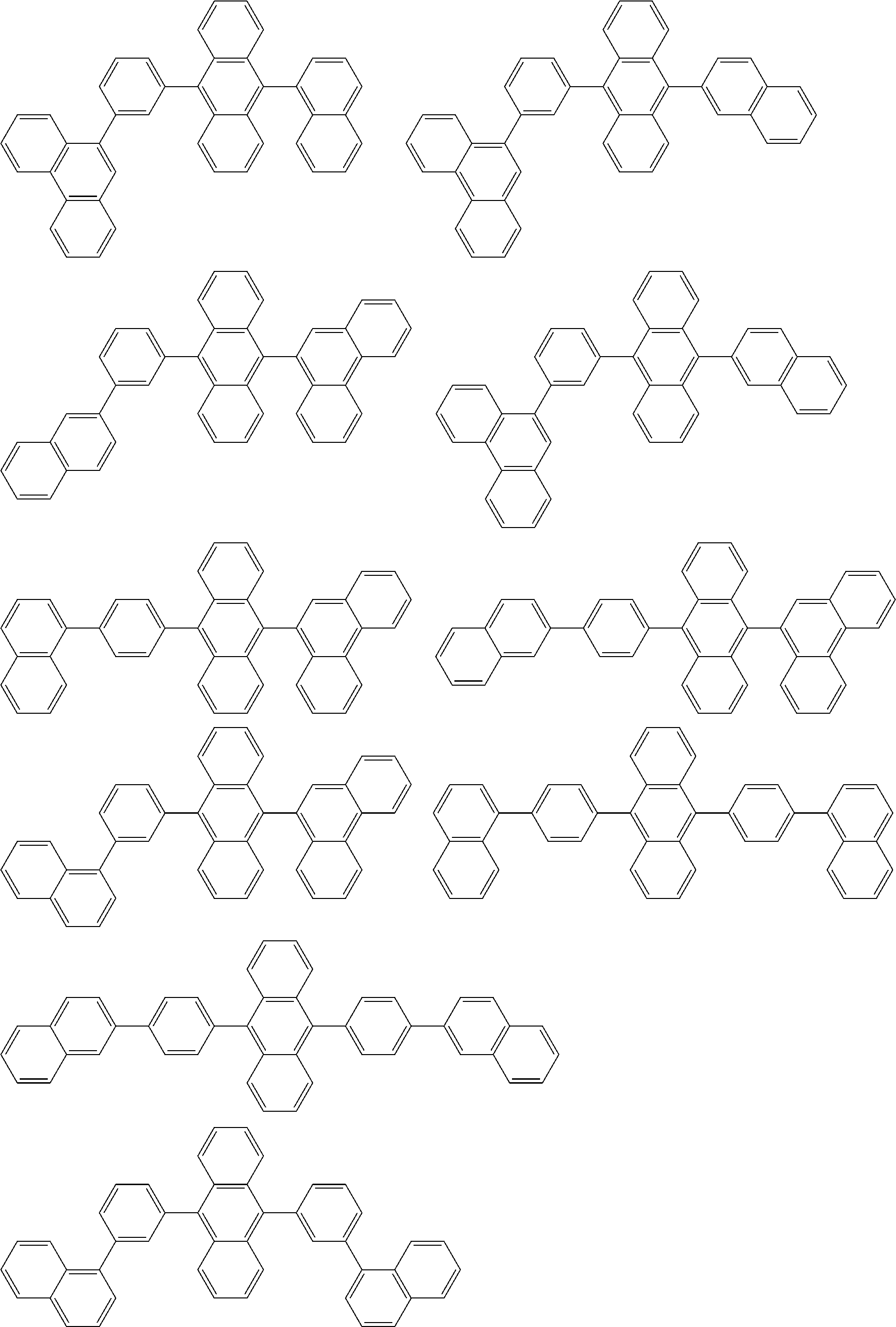

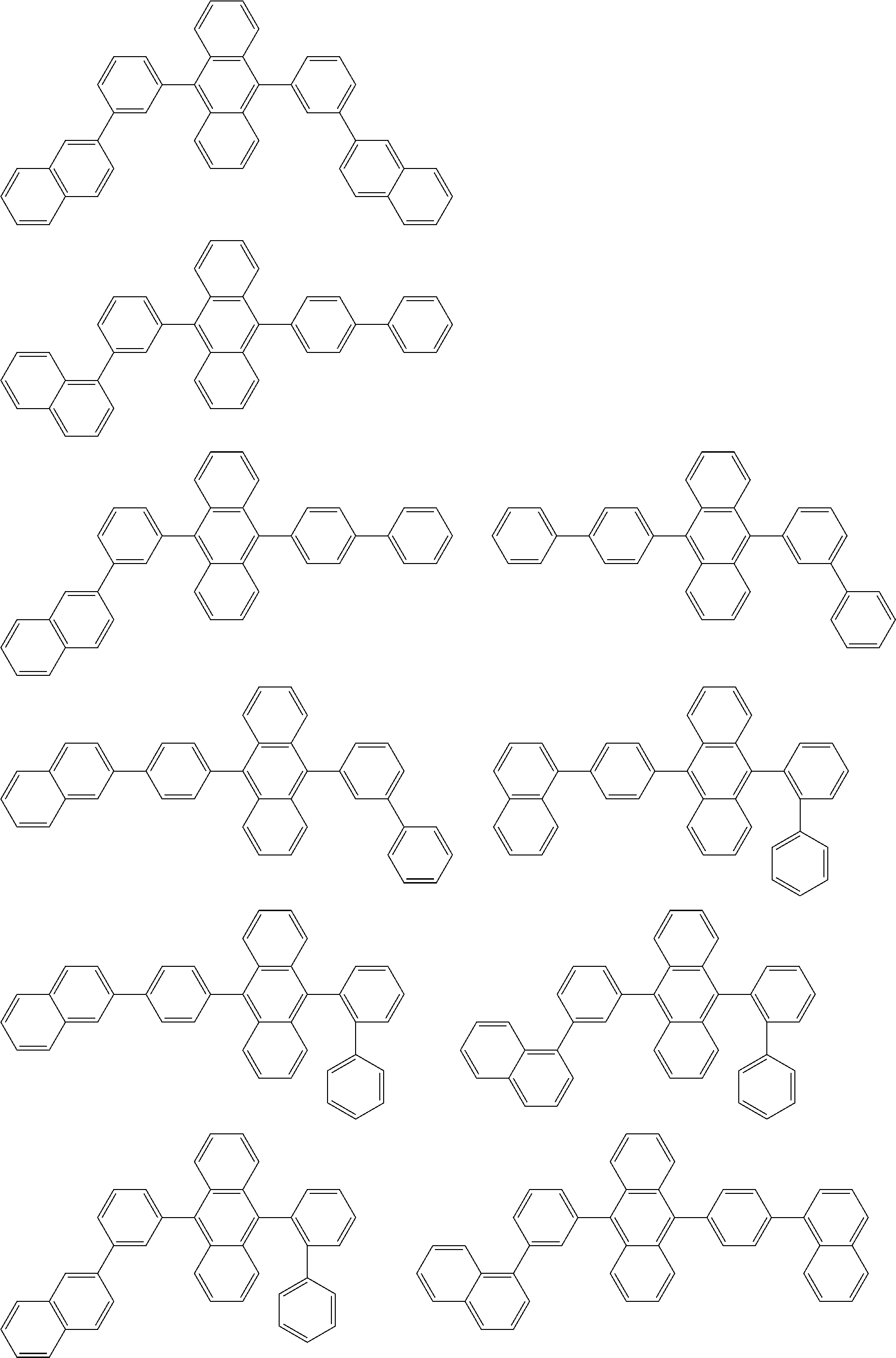

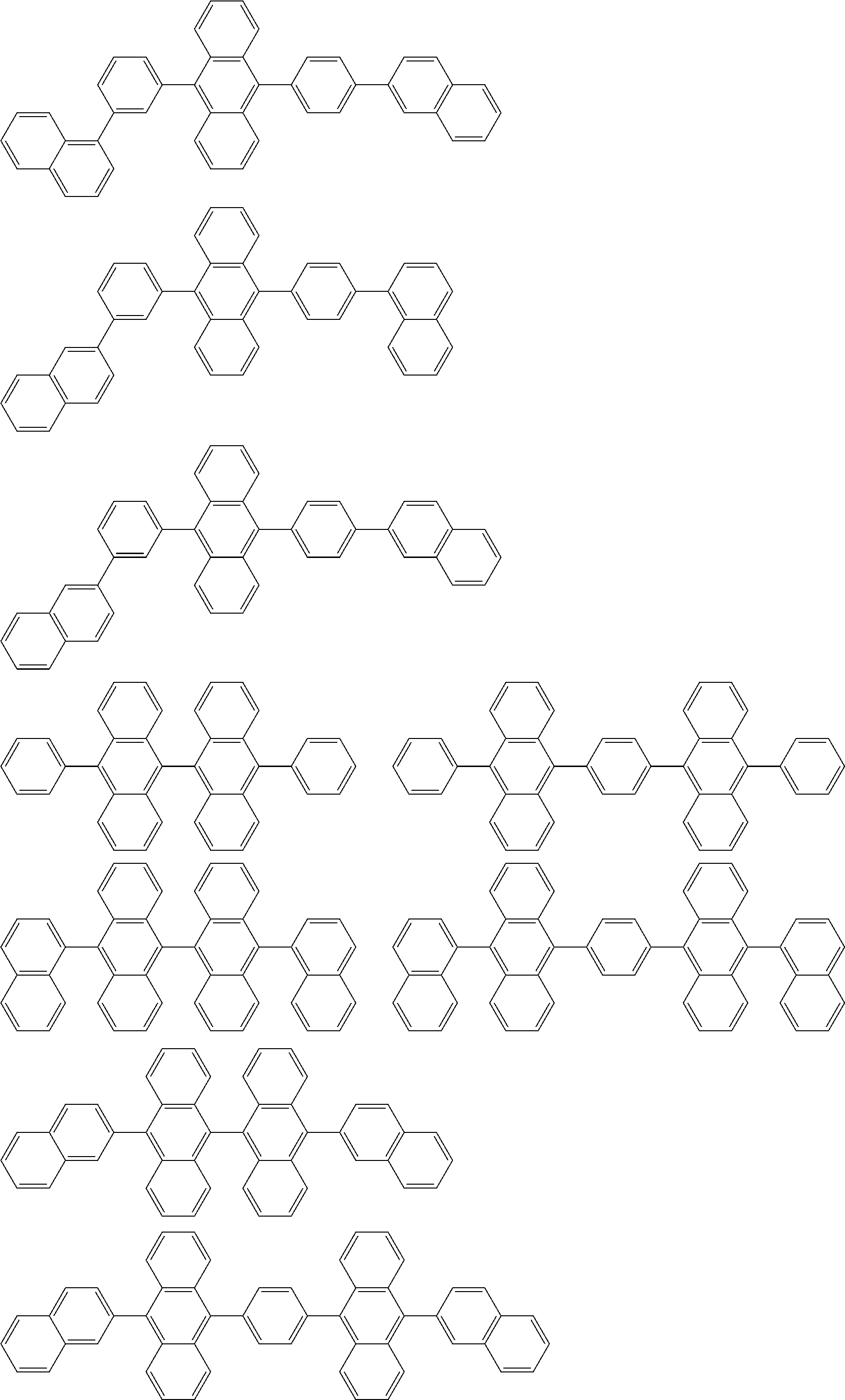

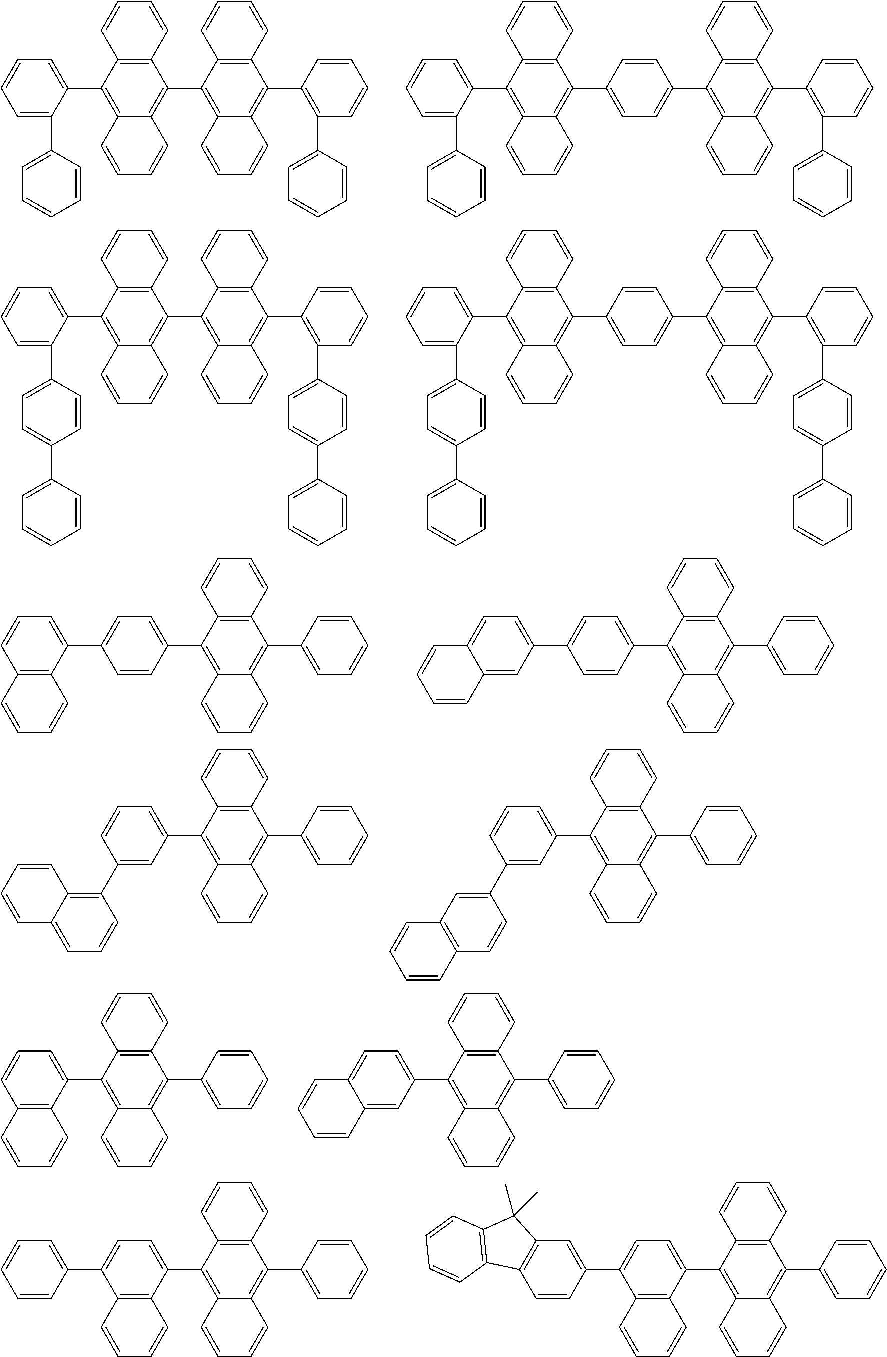

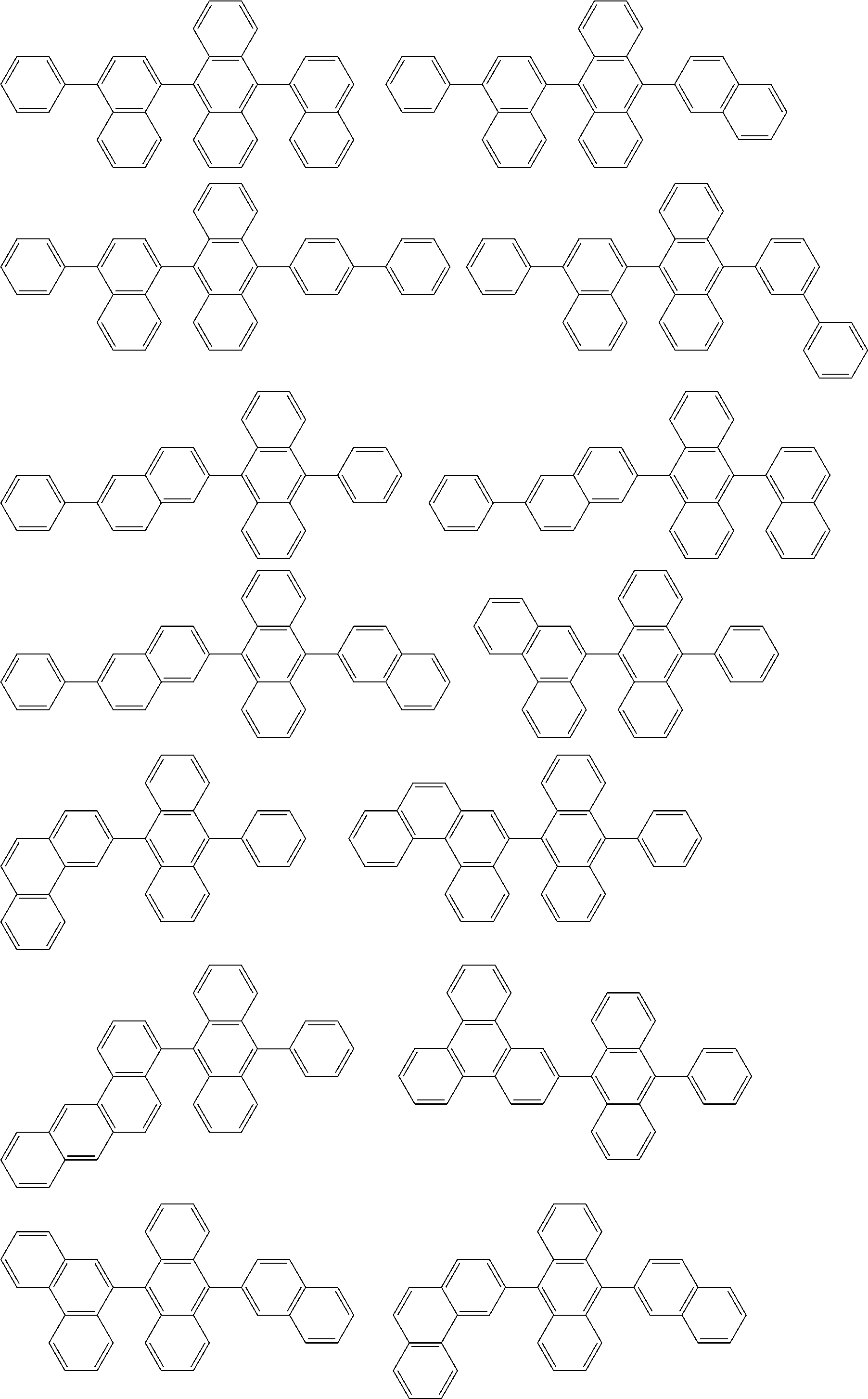

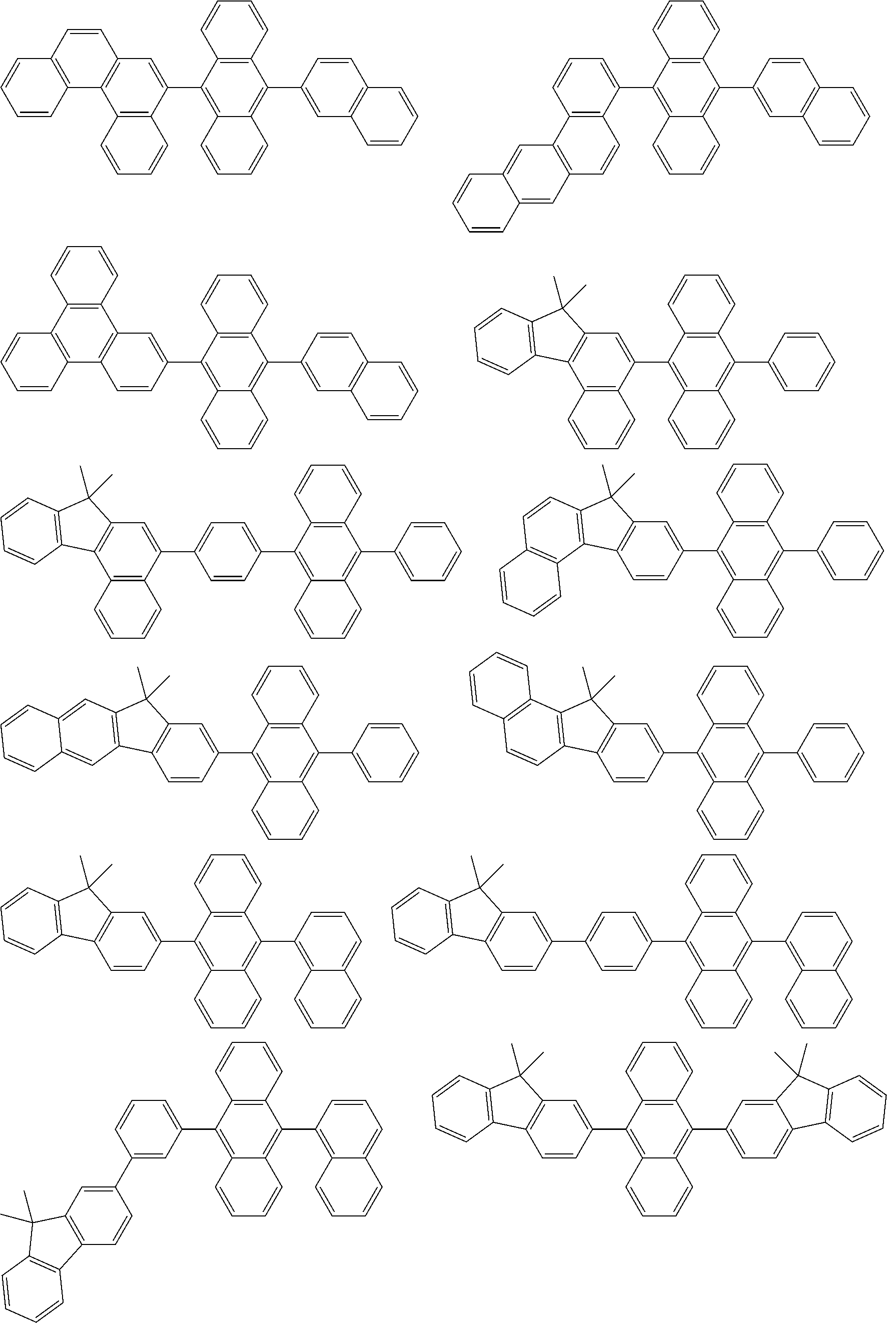

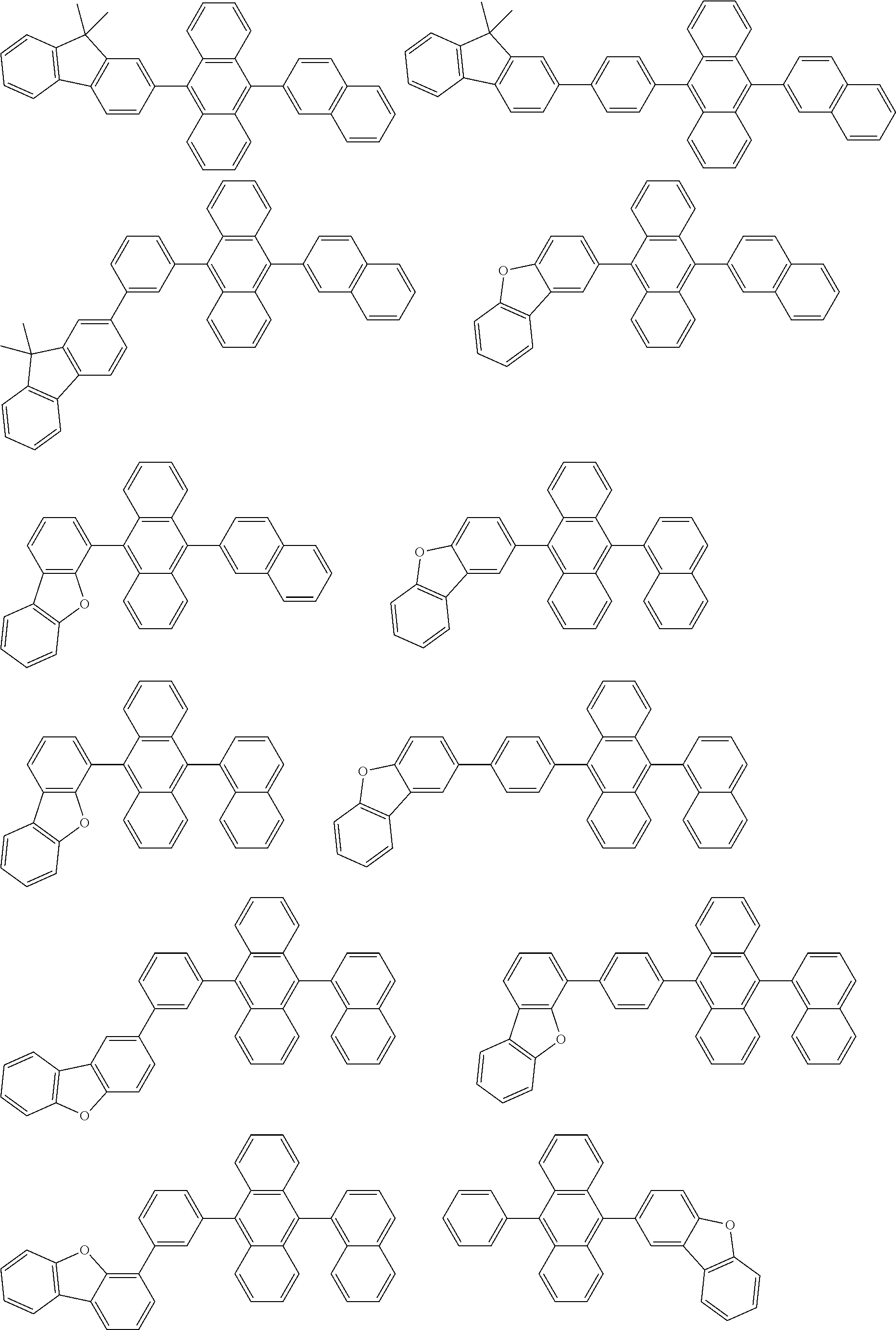

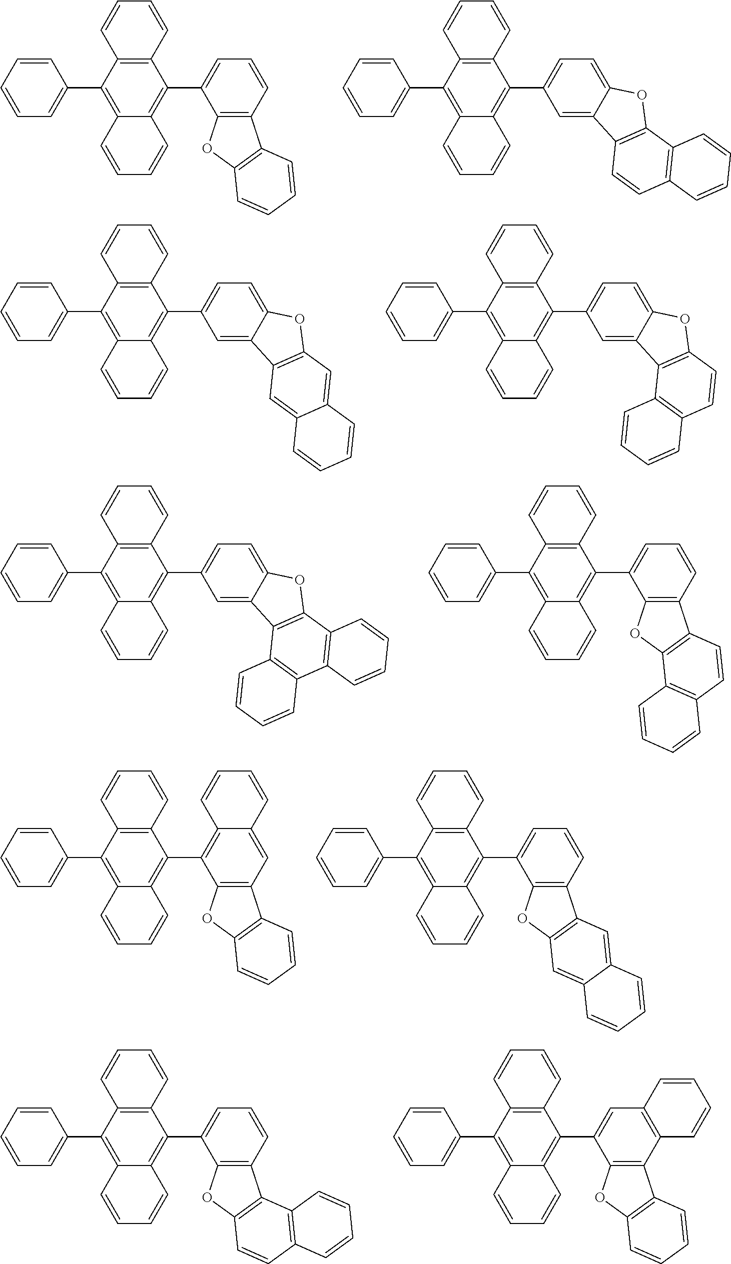

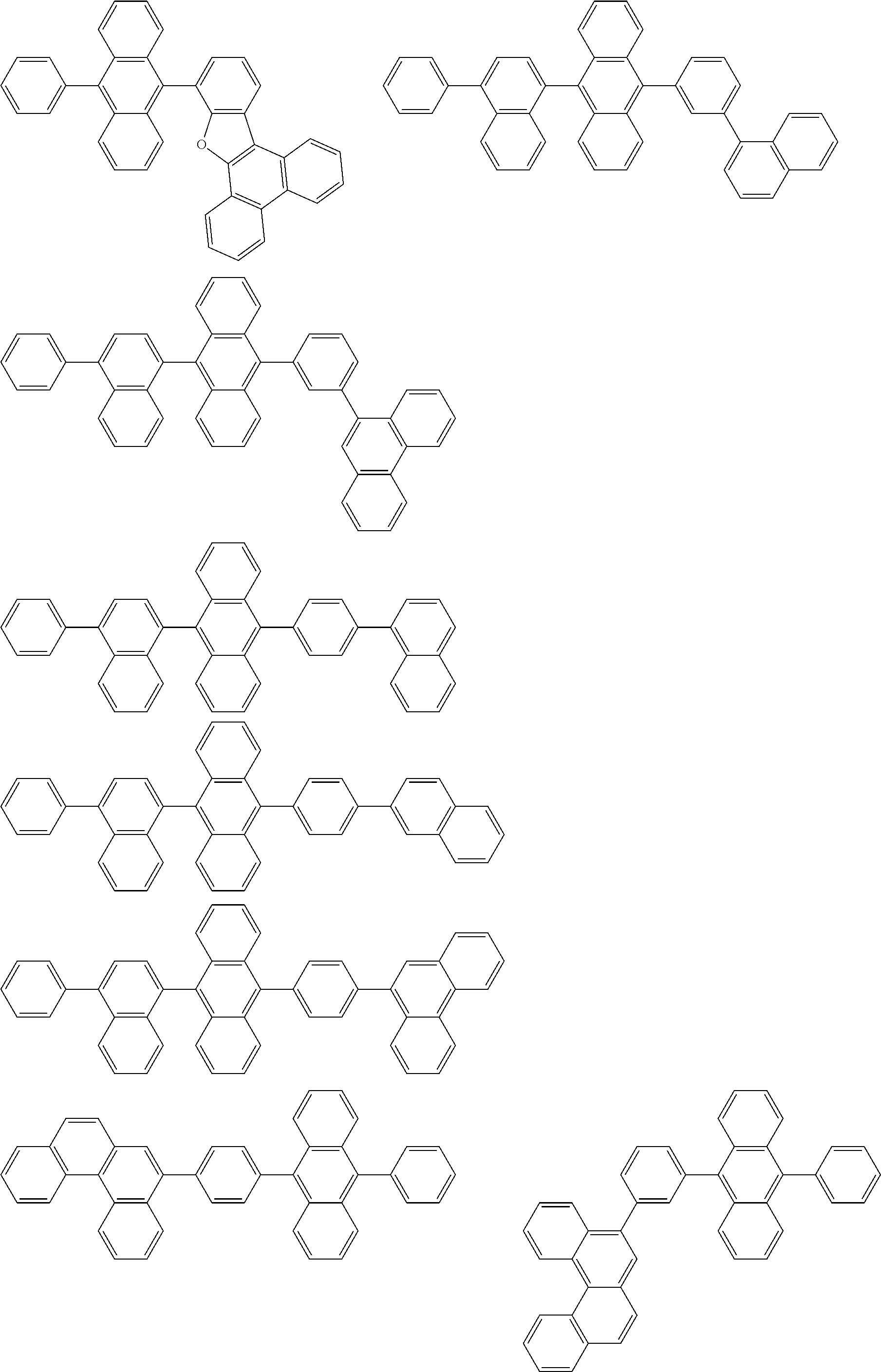

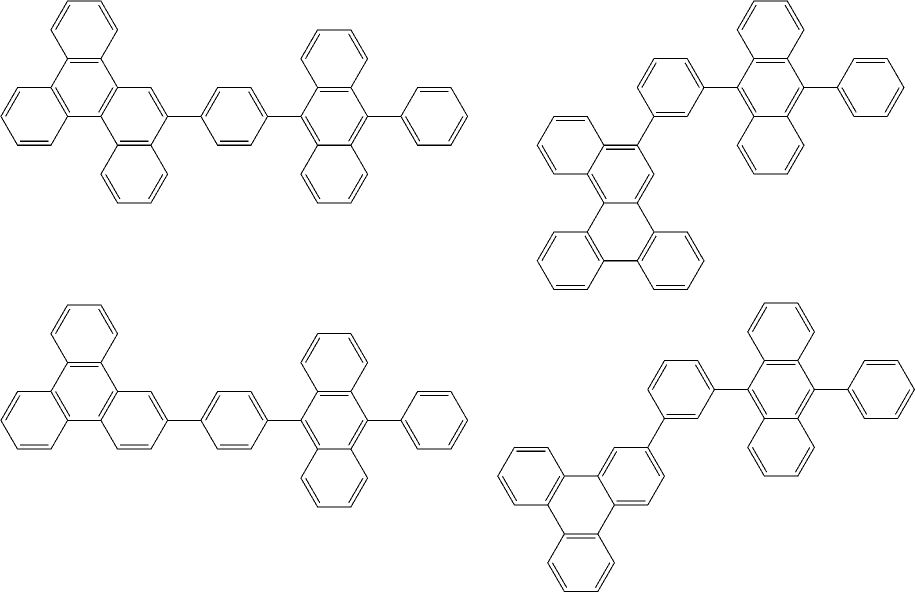

In one embodiment of the organic EL device according to the present invention, when the light emitting layer includes a first light emitting layer and a second light emitting layer, at least one of components constituting the first light emitting layer is different from a component constituting the second light emitting layer. For example, a dopant material contained in the first light emitting layer may be different from a dopant material contained in the second light emitting layer, or a host material contained in the first light emitting layer may be different from a host material contained in the second light emitting layer.

In the organic EL device according to the present embodiment, the light emitting layer may contain a light emitting compound exhibiting fluorescence emission having a main peak wavelength of 500 nm or less (hereinafter, also simply referred to as a “fluorescence emitting compound”).

The method for measuring the main peak wavelength of the compound is as follows. A 5 μmol/L toluene solution of a compound to be measured is prepared and placed in a quartz cell, and the emission spectrum (vertical axis: emission intensity, horizontal axis: wavelength) of the sample is measured at room temperature (300K). The emission spectrum can be measured using a fluorescence spectrophotometer (device name: F-7000) manufactured by Hitachi High-Tech Science Corporation. Note that the emission spectrum measuring device is not limited to the device used here.

In the emission spectrum, the peak wavelength of the emission spectrum at which the emission intensity becomes maximum is defined as a main peak wavelength. In the description herein, the main peak wavelength is sometimes referred to as a fluorescence emission main peak wavelength (FL-peak).

The fluorescence emitting compound may be the dopant material or the host material.

In the case where the light emitting layer is a single layer, only one of the dopant material and the host material may be the fluorescence emitting compound, or both of them may be the fluorescence emitting compound.

In addition, in the case where the light emitting layer includes a first light emitting layer (anode side) and a second light emitting layer (anode side), only one of the first light emitting layer and the second light emitting layer may include the fluorescence emitting compound, or both may include the fluorescence emitting compound. In the case where the first light emitting layer contains the fluorescence emitting compound, only one of the dopant material and the host material contained in the first light emitting layer may be the fluorescence emitting compound, or both of them may be the fluorescence emitting compound. In addition, in the case where the second light emitting layer contains the fluorescence emitting compound, only one of the dopant material and the host material contained in the second light emitting layer may be the fluorescence emitting compound, or both of them may be the fluorescence emitting compound.

### Electron Transporting Layer

The electron transporting layer is a layer containing a material having a high electron transporting capability (an electron transporting material) and is provided between the light emitting layer and the cathode, or between the electron injecting layer, if exists, and the light emitting layer.

The electron transporting layer may have a single layer structure or a multilayer structure including two or more layers. For example, the electron transporting layer may have a two layer structure including a first electron transporting layer (anode side) and a second electron transporting layer (cathode side). In one embodiment of the present invention, the electron transporting layer having a single layer structure is preferably disposed adjacent to the light emitting layer, and the electron transporting layer that is closest to the anode in the multilayer structure, such as the first electron transporting layer in the two layer structure, is preferably disposed adjacent to the light emitting layer. In another embodiment of the present invention, a hole blocking layer described later or the like may be interposed between the electron transporting layer having a single layer structure and the light emitting layer, or between the electron transporting layer that is closest to the light emitting layer in the multilayer structure and the light emitting layer.

As the electron transporting layer, for example, (1) a metal complex, such as an aluminum complex, a beryllium complex, and a zinc complex; (2) a heteroaromatic compound, such as an imidazole derivative, a benzimidazole derivative, an azine derivative, a carbazole derivative, and a phenanthroline derivative; and (3) a high-molecular weight compound can be used.

Examples of the metal complex include tris(8-quinolinolato)aluminum(III) (abbreviation: Alq), tris(4-methyl-8-quinolinolato)aluminum (abbreviation: Almq3), bis(10-hydroxybenzo[h]quinolinato)beryllium (abbreviation: BeBq2), bis(2-methyl-8-quinolinolato)(4-phenylphenolato)aluminum(III) (abbreviation: BAlq), bis(8-auinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ).

Examples of the heteroaromatic compound include 2-(4-biphenylyl)-5-(4-tert-butylphenyl)-1,3,4-oxadiazole (abbreviation: PBD), 1,3-bis[5-(p-tert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzxazol-2-yl)stilbene (abbreviation: BzOs).

Examples of the high-molecular weight compound include poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy).

The above-mentioned materials are materials having an electron mobility of 10⁻⁶ cm²/Vs or more. Materials other than those as mentioned above may also be used in the electron transporting layer so long as they are materials high in the electron transporting capability rather than in the hole transporting capability.

### Electron Injecting Layer

The electron injecting layer is a layer containing a material having a high electron injection capability. As the electron injecting layer, alkali metals, such as lithium (Li) and cesium (Cs), alkaline earth metals, such as magnesium (Mg), calcium (Ca), and strontium (Sr), rare earth metals, such as europium (Eu) and ytterbium (Yb), and compounds containing these metals can be used. Examples of the compounds include an alkali metal oxide, an alkali metal halide, an alkali metal-containing organic complex, an alkaline earth metal oxide, an alkaline earth metal halide, an alkaline earth metal-containing organic complex, a rare earth metal oxide, a rare earth metal halide, and a rare earth metal-containing organic complex. These compounds may be used as a mixture of a plurality thereof.

In addition, a material having an electron transporting capability, in which an alkali metal, an alkaline earth metal, or a compound thereof is contained, specifically Alq in which magnesium (Mg) is contained may be used. In this case, electron injection from the cathode can be more efficiently performed.

Otherwise, in the electron injecting layer, a composite material obtained by mixing an organic compound with an electron donor may be used. Such a composite material is excellent in the electron injection capability and the electron transporting capability because the organic compound receives electrons from the electron donor. In this case, the organic compound is preferably a material excellent in transporting received electrons, and specifically, examples thereof include a material constituting the aforementioned electron transporting layer (such as a metal complex and a heteroaromatic compound). As the electron donor, a material having an electron donation property for the organic compound may be used. Specifically, alkali metals, alkaline earth metals, and rare earth metals are preferred, and examples thereof include lithium, cesium, magnesium, calcium, erbium, and ytterbium. In addition, an alkali metal oxide or an alkaline earth metal oxide is preferred, and examples thereof include lithium oxide, calcium oxide, and barium oxide. In addition, a Lewis base, such as magnesium oxide, can also be used. In addition, an organic compound, such as tetrathiafulvalene (abbreviation: TTF), can also be used.

### Cathode

It is preferred that a metal, an alloy, an electrically conductive compound, or a mixture thereof which has a low work function (specifically 3.8 eV or less) is used for the cathode. Specific examples of such a cathode material include elements belonging to Group 1 or Group 2 of the periodic table of the elements, that is, alkali metals, such as lithium (Li) and cesium (Cs), alkaline earth metals, such as magnesium (Mg), calcium (Ca), and strontium (Sr), and alloys containing these (such as MgAg, and AlLi), and rare earth metals, such as europium (Eu), and ytterbium (Yb) and alloys containing these.

When the cathode is formed by using the alkali metals, the alkaline earth metals, and the alloys containing these, a vacuum vapor deposition method or a sputtering method can be adopted. In addition, when a silver paste or the like is used, a coating method, an inkjet method, or the like can be adopted.

By providing the electron injecting layer, the cathode can be formed using various conductive materials, such as Al, Ag, ITO, graphene, and indium oxide-tin oxide containing silicon or silicon oxide regardless of the magnitude of a work function. Such a conductive material can be deposited by using a sputtering method, an inkjet method, a spin coating method, or the like.

### Insulating Layer

The organic EL device applies an electric field to an ultrathin film, and thus, pixel defects are likely to occur due to leaks or short-circuiting. In order to prevent this, an insulating layer formed of an insulating thin film layer may be inserted between a pair of electrodes.

Examples of the material used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. A mixture or a laminate of these may also be used.

### Space Layer

The space layer is, for example, a layer provided between a fluorescent light emitting layer and a phosphorescent light emitting layer for the purpose of preventing excitons generated in the phosphorescent light emitting layer from diffusing into the fluorescent light emitting layer, or adjusting a carrier balance, in the case where the fluorescent light emitting layers and the phosphorescent light emitting layers are stacked. The space layer can also be provided among the plurality of phosphorescent light emitting layers.

Since the space layer is provided between the light emitting layers, a material having both an electron transporting capability and a hole transporting capability is preferred. Also, one having a triplet energy of 2.6 eV or more is preferred in order to prevent triplet energy diffusion in the adjacent phosphorescent light emitting layer. Examples of the material used for the space layer include the same as those used for the hole transporting layer as described above.

### Blocking Layer

The blocking layer such as the electron blocking layer, the hole blocking layer, or the exciton blocking layer may be provided adjacent to the light emitting layer. The electron blocking layer is a layer that prevents electrons from leaking from the light emitting layer to the hole transporting layer, and the hole blocking layer is a layer that prevents holes from leaking from the light emitting layer to the electron transporting layer. The exciton blocking layer has a function of preventing excitons generated in the light emitting layer from diffusing into the surrounding layers, and trapping the excitons within the light emitting layer.

Each layer of the organic EL device may be formed by a conventionally known vapor deposition method, a coating method, or the like. For example, formation can be performed by a known method using a vapor deposition method such as a vacuum vapor deposition method, or a molecular beam vapor deposition method (MBE method), or a coating method using a solution of a compound for forming a layer, such as a dipping method, a spin-coating method, a casting method, a bar-coating method, and a roll-coating method.

The film thickness of each layer is not particularly limited, but is typically 5 nm to 10 μm, and more preferably 10 nm to 0.2 μm because in general, when the film thickness is too small, defects such as pinholes are likely to occur, and conversely, when the film thickness is too large, a high driving voltage is required and the efficiency decreases.

In the organic EL device of the present invention having a hole transporting layer of a two layer structure or a three layer structure, the total of the thicknesses of the first hole transporting layer and the second hole transporting layer is preferably 30 nm or more and 150 nm or less, and more preferably 40 nm or more and 130 nm or less.

In one embodiment of the organic EL device of the present invention, the thickness of the second hole transporting layer is preferably 5 nm or more, more preferably 20 nm or more, still more preferably 25 nm or more, and particularly preferably 35 nm or more, and is preferably 100 nm or less.

In addition, in one embodiment of the organic EL device of the present invention, the thickness of the hole transporting layer adjacent to the light emitting layer is preferably 5 nm or more, more preferably 20 nm or more, still more preferably 25 nm or more, and particularly preferably 30 nm or more, and is preferably 100 nm or less.

In addition, in one embodiment of the organic EL device of the present invention, the thickness D1 of the first hole transporting layer and the thickness D2 of the second hole transporting layer satisfy the relationship of 0.3<D2/D1<4.0, preferably satisfy the relationship of 0.5<D2/D1<3.5, and more preferably satisfy the relationship 0.75<D2/D1<3.0.

Embodiments of the organic EL device of the present invention include, for example, the following:

### (1) Organic EL Device Having Hole Transporting Layer Having Two Layer Structure

a first embodiment in which the second hole transporting layer contains the inventive compound and the first hole transporting layer does not contain the inventive compound;
a second embodiment in which both the first hole transporting layer and the second hole transporting layer contain the inventive compound;
a third embodiment in which the first hole transporting layer contains the inventive compound and the second hole transporting layer does not contain the inventive compound;

### (2) Organic EL Device Having Hole Transporting Layer Having Three Layer Structure

a fourth embodiment in which the first hole transporting layer contains the inventive compound and the second and third hole transporting layers do not contain the inventive compound;
a fifth embodiment in which the second hole transporting layer contains the inventive compound and the first and third hole transporting layers do not contain the inventive compound;
a sixth embodiment in which the third hole transporting layer contains the inventive compound and the first and second hole transporting layers do not contain the inventive compound;
a seventh embodiment in which the first and second hole transporting layers contain the inventive compound and the third hole transporting layer does not contain the inventive compound;
an eighth embodiment in which the first and third hole transporting layers contain the inventive compound and the second hole transporting layer does not contain the inventive compound;
a tenth embodiment in which the second and third hole transporting layers contain the inventive compound and the first hole transporting layer does not contain the inventive compound; and
a tenth embodiment in which all of the first to third hole transporting layers contain the inventive compound.

### Electronic Device

The organic EL device can be used for electronic devices, such as display components of an organic EL panel module, display devices of a television, a mobile phone and a personal computer, and light emitting devices of lightings and vehicular lamps.

### EXAMPLES

The present invention is hereunder described in more detail by reference to Examples, but it should be construed that the present invention is not limited to the following Examples.

Inventive Compounds used for Production of Organic EL devices of Examples 1 to 17

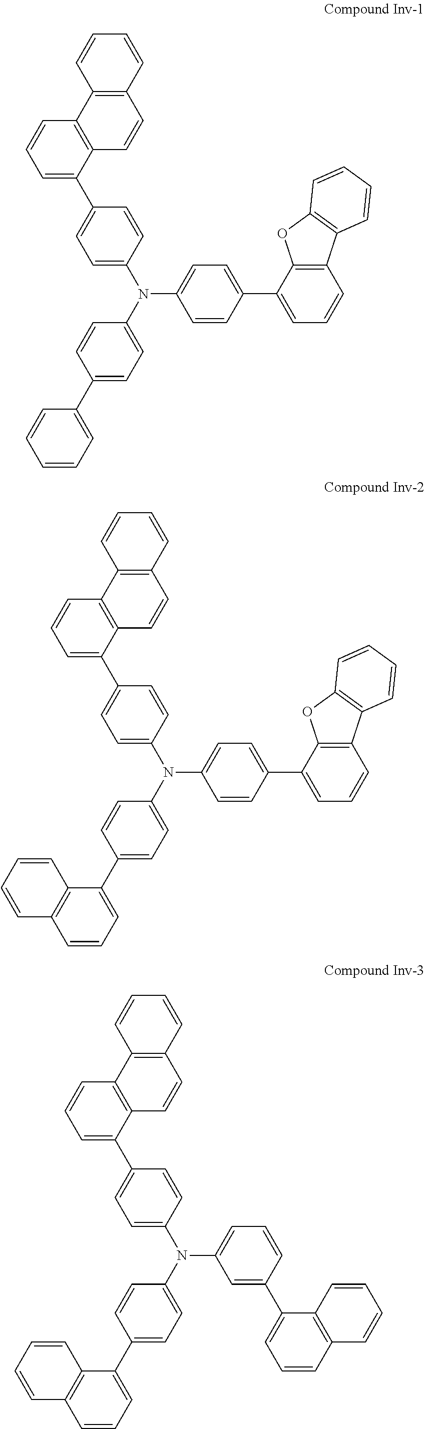 [SMILES: c1ccc(-c2ccc(N(c3ccc(-c4cccc5c4ccc4ccccc45)cc3)c3ccc(-c4cccc5c4oc4ccccc45)cc3)cc2)cc1 ; c1ccc2c(-c3ccc(N(c4ccc(-c5cccc6c5ccc5ccccc56)cc4)c4ccc(-c5cccc6c5oc5ccccc56)cc4)cc3)cccc2c1 ; c1cc(-c2cccc3ccccc23)cc(N(c2ccc(-c3cccc4ccccc34)cc2)c2ccc(-c3cccc4c3ccc3ccccc34)cc2)c1]

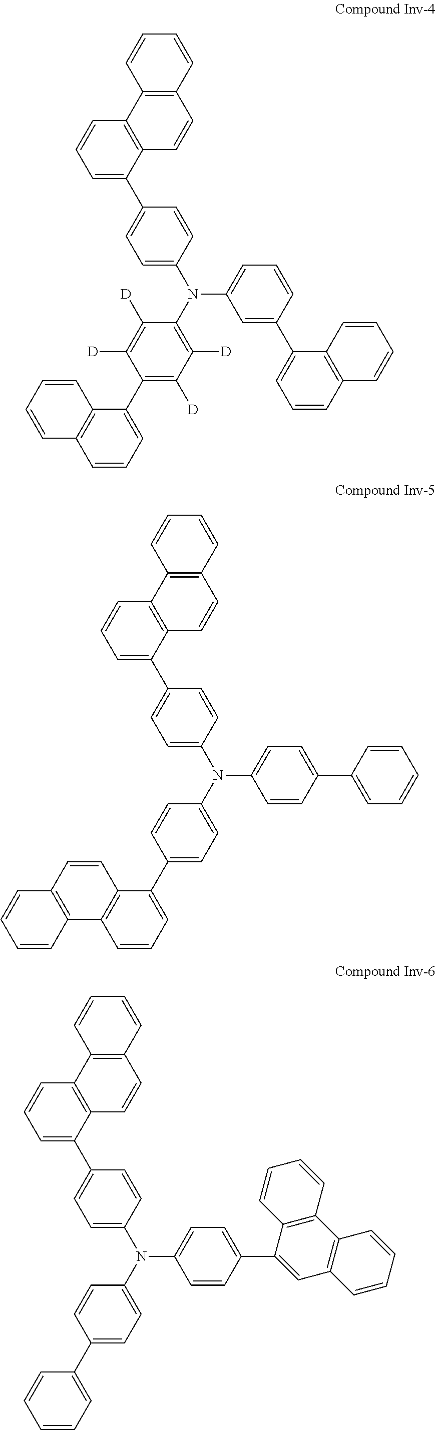 [SMILES: [2H]c1c([2H])c(N(c2ccc(-c3cccc4c3ccc3ccccc34)cc2)c2cccc(-c3cccc4ccccc34)c2)c([2H])c([2H])c1-c1cccc2ccccc12 ; c1ccc(-c2ccc(N(c3ccc(-c4cccc5c4ccc4ccccc45)cc3)c3ccc(-c4cccc5c4ccc4ccccc45)cc3)cc2)cc1 ; c1ccc(-c2ccc(N(c3ccc(-c4cccc5c4ccc4ccccc45)cc3)c3ccc(-c4cc5ccccc5c5ccccc45)cc3)cc2)cc1]

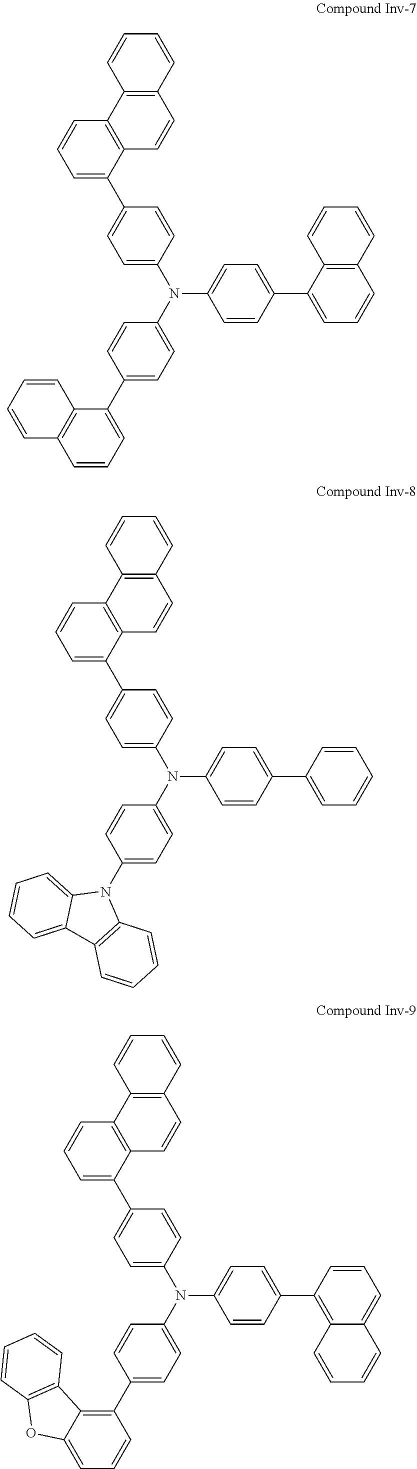 [SMILES: c1ccc2c(-c3ccc(N(c4ccc(-c5cccc6ccccc56)cc4)c4ccc(-c5cccc6c5ccc5ccccc56)cc4)cc3)cccc2c1 ; c1ccc(-c2ccc(N(c3ccc(-c4cccc5c4ccc4ccccc45)cc3)c3ccc(-n4c5ccccc5c5ccccc54)cc3)cc2)cc1 ; c1ccc2c(-c3ccc(N(c4ccc(-c5cccc6c5ccc5ccccc56)cc4)c4ccc(-c5cccc6oc7ccccc7c56)cc4)cc3)cccc2c1]

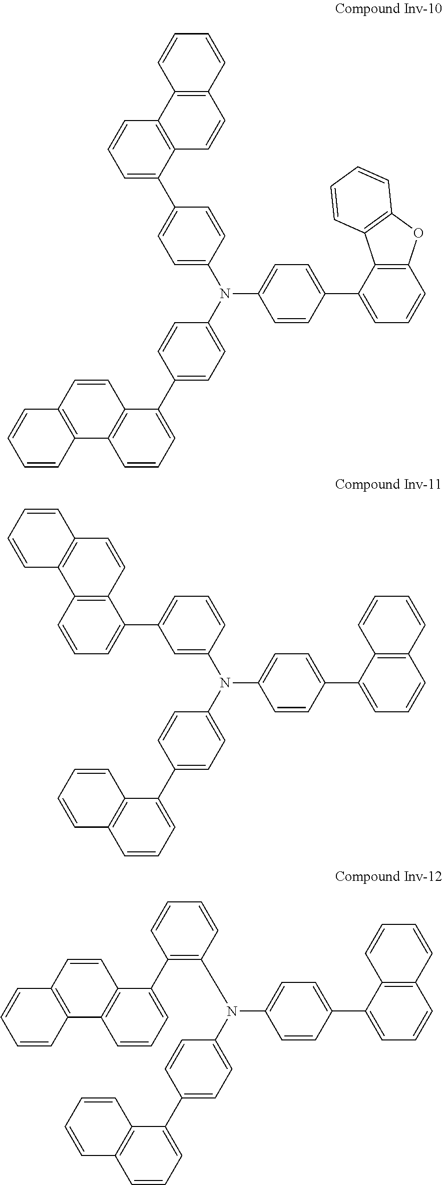

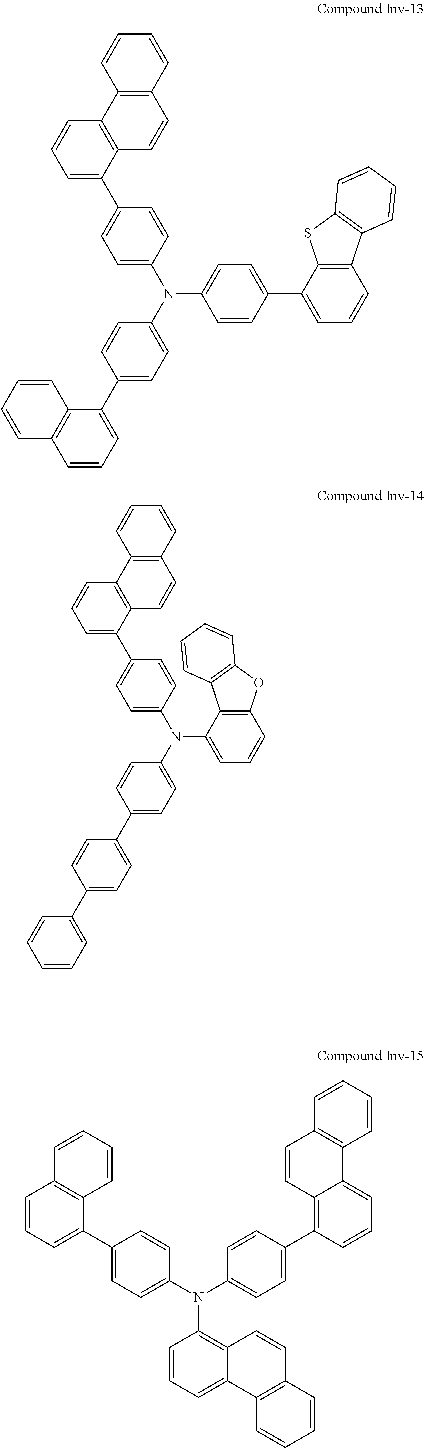 [SMILES: c1ccc2c(-c3ccc(N(c4ccc(-c5cccc6c5ccc5ccccc56)cc4)c4ccc(-c5cccc6c5sc5ccccc56)cc4)cc3)cccc2c1 ; c1ccc(-c2ccc(-c3ccc(N(c4ccc(-c5cccc6c5ccc5ccccc56)cc4)c4cccc5oc6ccccc6c45)cc3)cc2)cc1 ; c1ccc2c(-c3ccc(N(c4ccc(-c5cccc6c5ccc5ccccc56)cc4)c4cccc5c4ccc4ccccc45)cc3)cccc2c1]

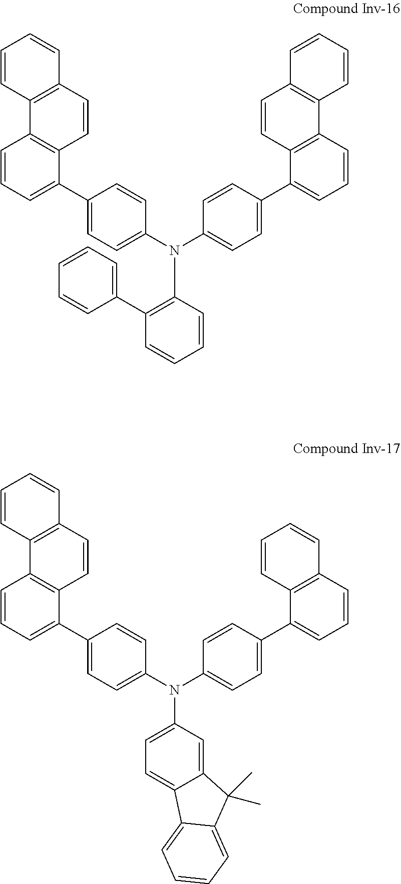 [SMILES: c1ccc(-c2ccccc2N(c2ccc(-c3cccc4c3ccc3ccccc34)cc2)c2ccc(-c3cccc4c3ccc3ccccc34)cc2)cc1 ; CC1(C)c2ccccc2-c2ccc(N(c3ccc(-c4cccc5ccccc45)cc3)c3ccc(-c4cccc5c4ccc4ccccc45)cc3)cc21]

Comparative Compounds used for Production of Organic EL devices of Comparative Examples 1 to 4

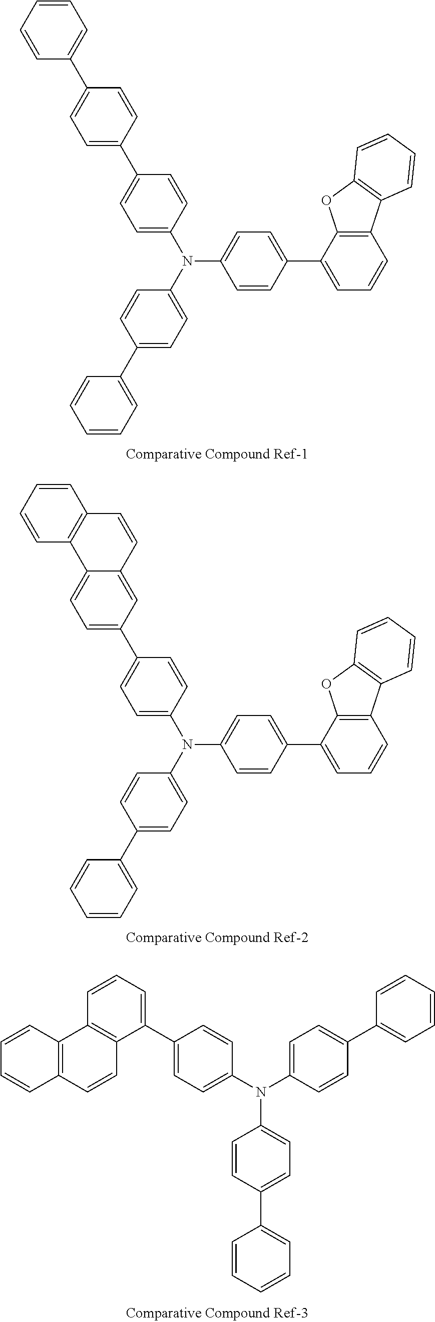 [SMILES: c1ccc(-c2ccc(-c3ccc(N(c4ccc(-c5ccccc5)cc4)c4ccc(-c5cccc6c5oc5ccccc56)cc4)cc3)cc2)cc1 ; c1ccc(-c2ccc(N(c3ccc(-c4ccc5c(ccc6ccccc65)c4)cc3)c3ccc(-c4cccc5c4oc4ccccc45)cc3)cc2)cc1 ; c1ccc(-c2ccc(N(c3ccc(-c4ccccc4)cc3)c3ccc(-c4cccc5c4ccc4ccccc45)cc3)cc2)cc1]

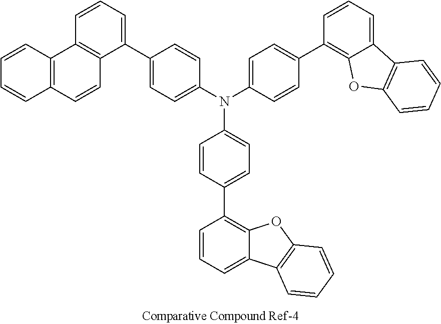 [SMILES: c1ccc2c(c1)ccc1c(-c3ccc(N(c4ccc(-c5cccc6c5oc5ccccc56)cc4)c4ccc(-c5cccc6c5oc5ccccc56)cc4)cc3)cccc12]

Other Compounds used for Production of Organic EL devices of Examples 1 to 17 and Comparative Examples 1 to 4

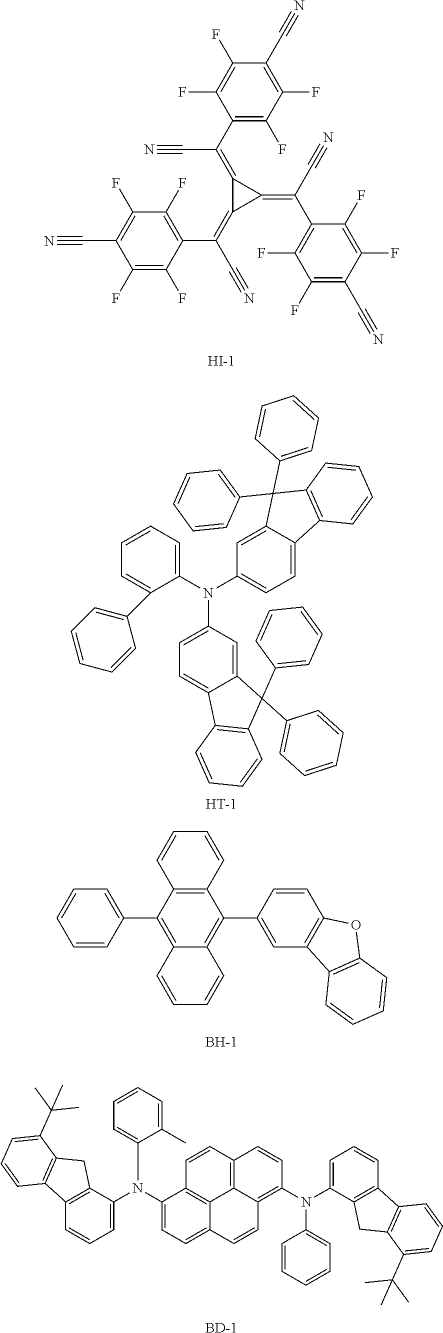

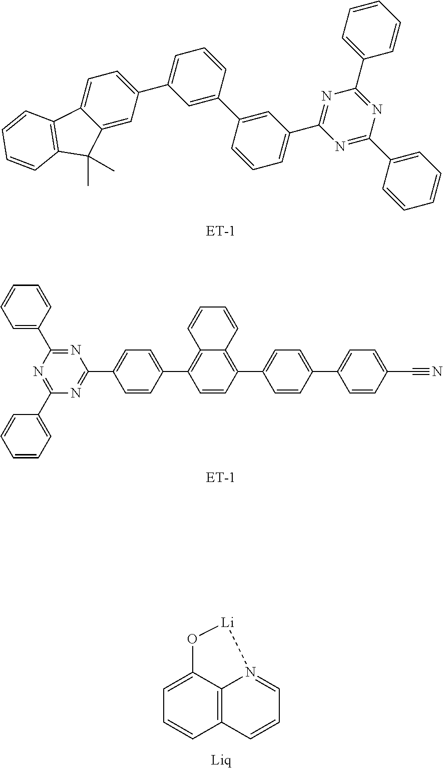

### Production of Organic EL Device (I)

### Example 1

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the ITO transparent electrode was mounted on a substrate holder of a vacuum vapor deposition apparatus, and firstly, Compound HT-1 and Compound HI-1 were vapor co-deposited on the surface having the transparent electrode formed thereon, so as to cover the transparent electrode, resulting in a hole injecting layer with a film thickness of 10 nm. The mass ratio of Compound HT-1 to Compound HI-1 (HT-1:HI-1) was 97:3.

Subsequently, on this hole injecting layer, Compound HT-1 was vapor deposited to form a first hole transporting layer with a film thickness of 80 nm.

Subsequently, on this first hole transporting layer, Compound Inv-1 was vapor deposited to form a second hole transporting layer with a film thickness of 10 nm.

Subsequently, on this second hole transporting layer, Compound BH-1 (host material) and Compound BD-1 (dopant material) were vapor co-deposited to form a light emitting layer with a film thickness of 25 nm. The mass ratio of Compound BH-1 to Compound BD-1 (BH-1:BD-1) was 96:4.

Subsequently, on this light emitting layer, Compound ET-1 was vapor deposited to form a first electron transporting layer with a film thickness of 5 nm.

Subsequently, on this first electron transporting layer, Compound ET-2 and Liq were vapor co-deposited to form a second electron transporting layer with a film thickness of 20 nm. The mass ratio of Compound ET-2 to Liq (ET-2:Liq) was 50:50.

Next, LiF was vapor deposited on this second electron transporting layer to form an electron injecting electrode having a film thickness of 1 nm.

Next, metal Al was vapor deposited on the electron injecting electrode to form a metal cathode having a film thickness of 50 nm.

The layer configuration of the organic EL device of Example 1 thus obtained is shown below.

### ITO (130)/(HT-1:HI-=97:3) (10)/HT-1 (80)/Compound Inv-1 (10)/(BH-1:BD-1=96:4) (25)/ET-1 (5)/(ET-2:Liq=50:50) (20)/LiF (1)/Al (50)

In the layer configuration, the numeral in parentheses indicates the film thickness (nm), and the ratio is a mass ratio.

### Examples 2 to 17 and Comparative Examples 1 to 4

Each organic EL device was produced in the same manner as in Example 1 except that the compound shown in Table 1 was used instead of Compound Inv-1, and the external quantum efficiency was measured. The results are shown in Table 1.

### Measurement of External Quantum Efficiency (EQE)

The obtained organic EL devices were DC constant current driven at a current density of 10 mA/cm² at room temperature. The luminance was measured using a luminance meter (spectral luminance radiometer CS-1000 manufactured by Minolta Co., Ltd.), and the external quantum efficiency (%) was obtained from the result. The results are shown in Table 1.

TABLE 1

| | Second hole | |
|---|---|---|
| | transporting layer | EQE (%) |
| | material | @10 mA/cm² |
| Example 1 | Compound Inv-1 | 8.30 |
| Example 2 | Compound Inv-2 | 8.36 |
| Example 3 | Compound Inv-3 | 8.71 |
| Example 4 | Compound Inv-4 | 8.71 |
| Example 5 | Compound Inv-5 | 8.30 |
| Example 6 | Compound Inv-6 | 8.32 |
| Example 7 | Compound Inv-7 | 8.46 |
| Example 8 | Compound Inv-8 | 8.36 |
| Example 9 | Compound Inv-9 | 8.56 |
| Example 10 | Compound Inv-10 | 8.37 |
| Example 11 | Compound Inv-11 | 8.58 |
| Example 12 | Compound Inv-12 | 8.32 |
| Example 13 | Compound Inv-13 | 8.44 |
| Example 14 | Compound Inv-14 | 9.03 |
| Example 15 | Compound Inv-15 | 8.30 |
| Example 16 | Compound Inv-16 | 8.58 |
| Example 17 | Compound Inv-17 | 8.53 |
| Comparative | Comparative | 7.19 |
| Example 1 | Compound Ref-1 | |
| Comparative | Comparative | 7.74 |
| Example 2 | Compound Ref-2 | |
| Comparative | Comparative | 7.89 |
| Example 3 | Compound Ref-3 | |
| Comparative | Comparative | 7.85 |
| Example 4 | Compound Ref-4 | |

As is clear from the results of Table 1, the monoamines satisfying the requirements of the present invention (the compounds of Examples 1 to 17) provide organic EL devices having improved external quantum efficiency as compared with the monoamines not satisfying the requirements of the present invention (the comparative compounds of Comparative Examples 1 to 4).

Inventive Compounds used for Production of Organic EL devices of Examples 18 to 22

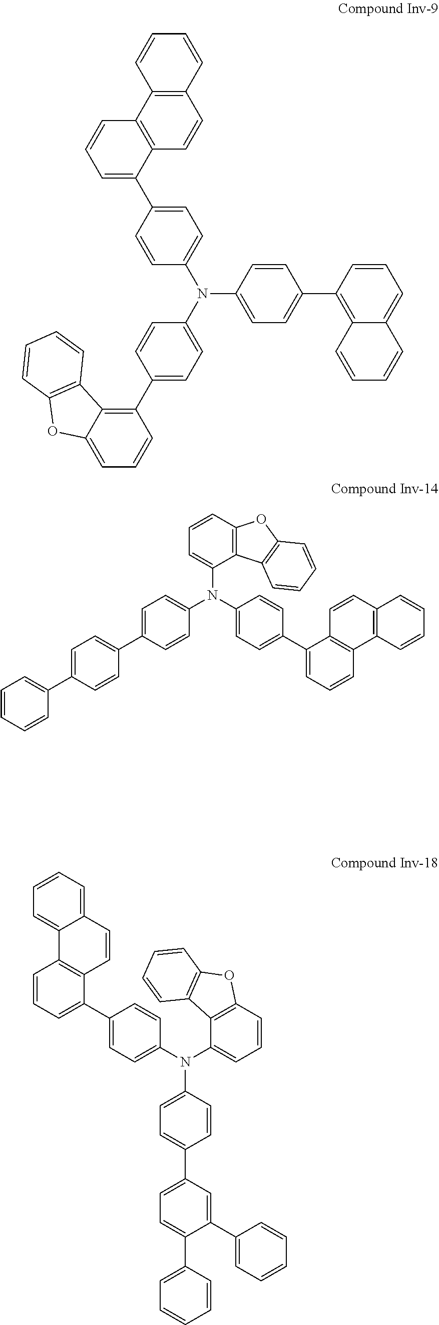 [SMILES: c1ccc2c(-c3ccc(N(c4ccc(-c5cccc6c5ccc5ccccc56)cc4)c4ccc(-c5cccc6oc7ccccc7c56)cc4)cc3)cccc2c1 ; c1ccc(-c2ccc(-c3ccc(N(c4ccc(-c5cccc6c5ccc5ccccc56)cc4)c4cccc5oc6ccccc6c45)cc3)cc2)cc1 ; c1ccc(-c2ccc(-c3ccc(N(c4ccc(-c5cccc6c5ccc5ccccc56)cc4)c4cccc5oc6ccccc6c45)cc3)cc2-c2ccccc2)cc1]

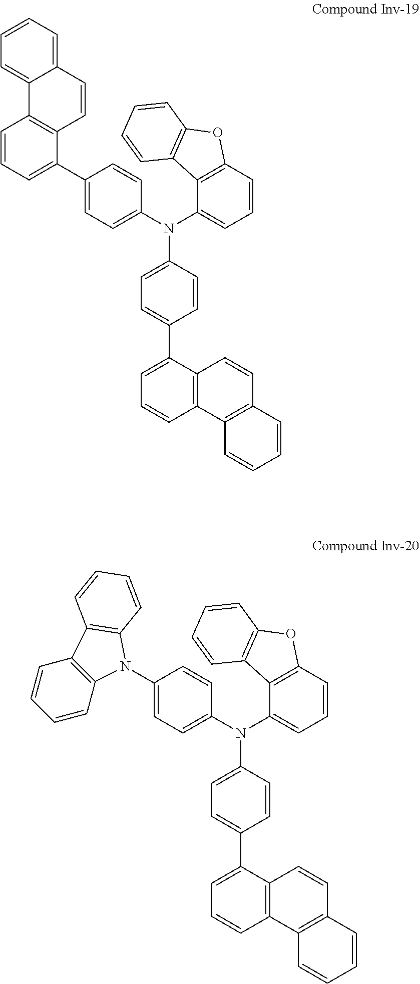 [SMILES: c1ccc2c(c1)ccc1c(-c3ccc(N(c4ccc(-c5cccc6c5ccc5ccccc56)cc4)c4cccc5oc6ccccc6c45)cc3)cccc12 ; c1ccc2c(c1)ccc1c(-c3ccc(N(c4ccc(-n5c6ccccc6c6ccccc65)cc4)c4cccc5oc6ccccc6c45)cc3)cccc12]

Comparative Compounds used for Production of Organic EL devices of Comparative Examples 5 and 6

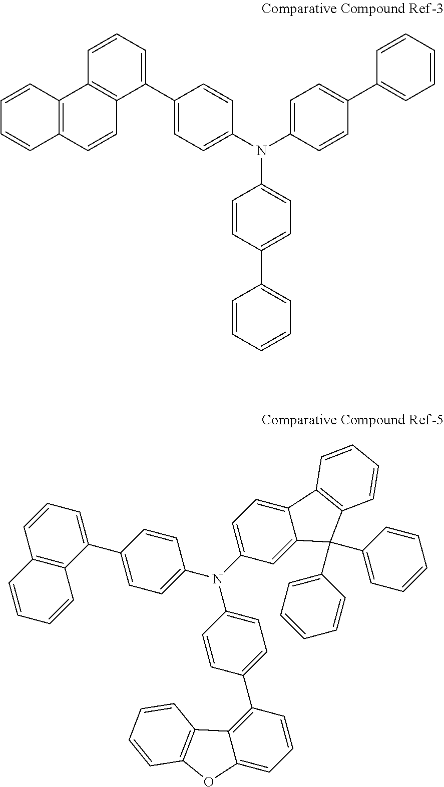 [SMILES: c1ccc(-c2ccc(N(c3ccc(-c4ccccc4)cc3)c3ccc(-c4cccc5c4ccc4ccccc45)cc3)cc2)cc1 ; c1ccc(C2(c3ccccc3)c3ccccc3-c3ccc(N(c4ccc(-c5cccc6ccccc56)cc4)c4ccc(-c5cccc6oc7ccccc7c56)cc4)cc32)cc1]

Other Compounds used for Production of Organic EL devices of Examples 18 to 22 and Comparative Examples 5 and 6

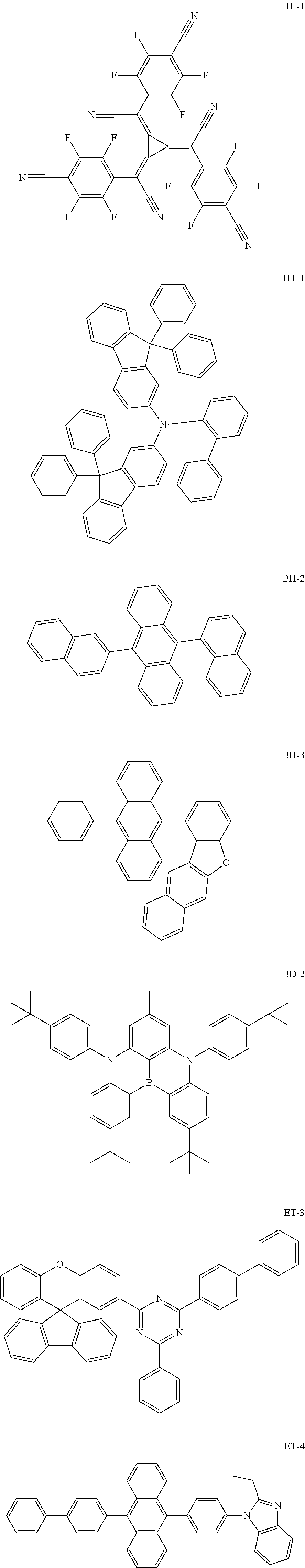

### Production of Organic EL Device (II)

### Example 18

A glass substrate of 25 mm×75 mm×1.1 mm provided with an ITO transparent electrode (anode) (manufactured by GEOMATEC Co., Ltd.) was ultrasonically cleaned in isopropyl alcohol for 5 minutes and then subjected to UV ozone cleaning for 30 minutes. The film thickness of the ITO was 130 nm.

The cleaned glass substrate provided with the ITO transparent electrode was mounted on a substrate holder of a vacuum vapor deposition apparatus, and firstly, Compound HT-1 and Compound HI-1 were vapor co-deposited on the surface having the transparent electrode formed thereon, so as to cover the transparent electrode, resulting in a hole injecting layer with a film thickness of 10 nm. The mass ratio of Compound HT-1 to Compound HI-1 (HT-1:HI-1) was 97:3.

Subsequently, on this hole injecting layer, Compound HT-1 was vapor deposited to form a first hole transporting layer with a film thickness of 75 nm.

Subsequently, on this first hole transporting layer, Compound Inv-9 was vapor deposited to form a second hole transporting layer with a film thickness of 7.5 nm.

Subsequently, on this second hole transporting layer, Compound BH-2 (host material), Compound BH-3 (host material), and Compound BD-2 (dopant material) were vapor co-deposited to form a light emitting layer with a film thickness of 20 nm. The mass ratio of Compound BH-2, Compound BH-3, and Compound BD-2 contained in the light emitting layer (BH-2:BH-3:BD-2) was 60:40:2.

Subsequently, on this light emitting layer, Compound ET-3 was vapor deposited to form a first electron transporting layer with a film thickness of 3 nm.

Subsequently, on this first electron transporting layer, Compound ET-4 and Liq were vapor co-deposited to form a second electron transporting layer with a film thickness of 30 nm. The mass ratio of Compound ET-4 to Liq (ET-4:Liq) was 50:50.

Next, LiF and Yb were vapor co-deposited on this second electron transporting layer to form an electron injecting electrode having a film thickness of 1 nm. The mass ratio of LiF to Yb (LiF:Yb) was 50:50.

Next, metal Al was vapor deposited on the electron injecting electrode to form a metal cathode having a film thickness of 50 nm.

The layer configuration of the organic EL device of Example 1 thus obtained is shown below.

### ITO (130)/(HT-1:HA=97:3) (10)/HT-1 (75)/Compound Inv-9 (7.5)/(BH-2:BH-3:BD-2=60:40:2) (20)/ET-3 (3)/(ET-4:Liq=50:50) (30)/(LiF:Yb=50:50) (1)/Al (50)

In the layer configuration, the numeral in parentheses indicates the film thickness (nm), and the ratio is a mass ratio.

The results of measuring the external quantum efficiency in the same manner as in Example 1 are shown in Table 2.

### Examples 19 to 22 and Comparative Examples 5 and 6

Each organic EL device was produced in the same manner as in Example 18 except that the compound shown in Table 2 was used instead of Compound Inv-9, and the external quantum efficiency was measured in the same manner as in Example 1. The results are shown in Table 2.

TABLE 2

| | Second hole | |
|---|---|---|
| | transporting layer | EQE (%) |
| | material | @10 mA/cm² |
| Example 18 | Compound Inv-9 | 10.41 |
| Example 19 | Compound Inv-14 | 10.25 |
| Example 20 | Compound Inv-18 | 10.26 |
| Example 21 | Compound Inv-19 | 10.40 |
| Example 22 | Compound Inv-20 | 10.44 |
| Comparative | Comparative | 9.50 |
| Example 5 | Compound Ref-3 | |
| Comparative | Comparative | 9.72 |
| Example 6 | Compound Ref-5 | |

### INVENTIVE COMPOUNDS SYNTHESIZED IN SYNTHESIS EXAMPLES

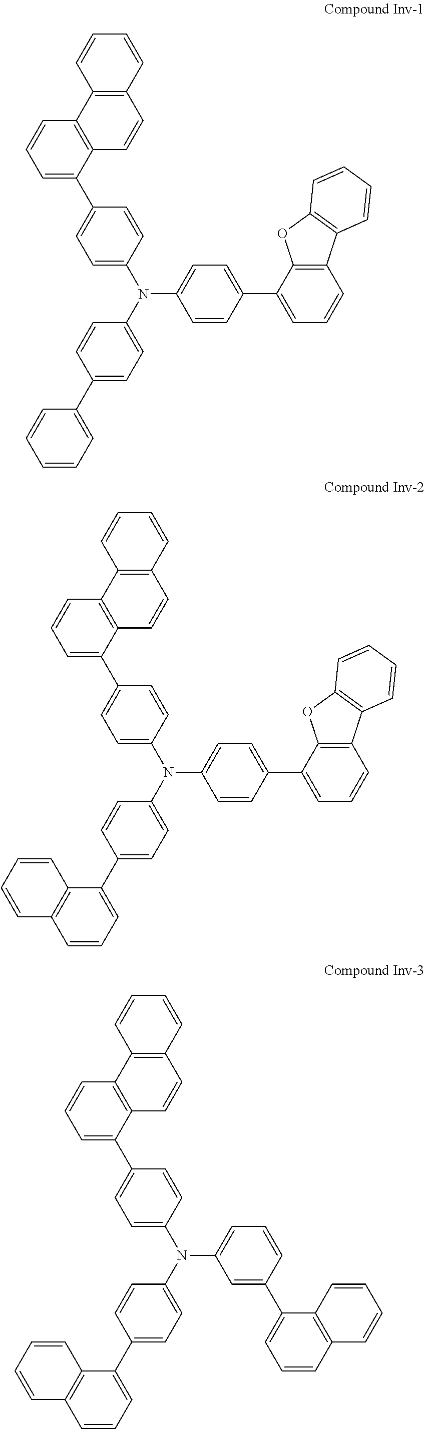 [SMILES: c1ccc(-c2ccc(N(c3ccc(-c4cccc5c4ccc4ccccc45)cc3)c3ccc(-c4cccc5c4oc4ccccc45)cc3)cc2)cc1 ; c1ccc2c(-c3ccc(N(c4ccc(-c5cccc6c5ccc5ccccc56)cc4)c4ccc(-c5cccc6c5oc5ccccc56)cc4)cc3)cccc2c1 ; c1cc(-c2cccc3ccccc23)cc(N(c2ccc(-c3cccc4ccccc34)cc2)c2ccc(-c3cccc4c3ccc3ccccc34)cc2)c1]

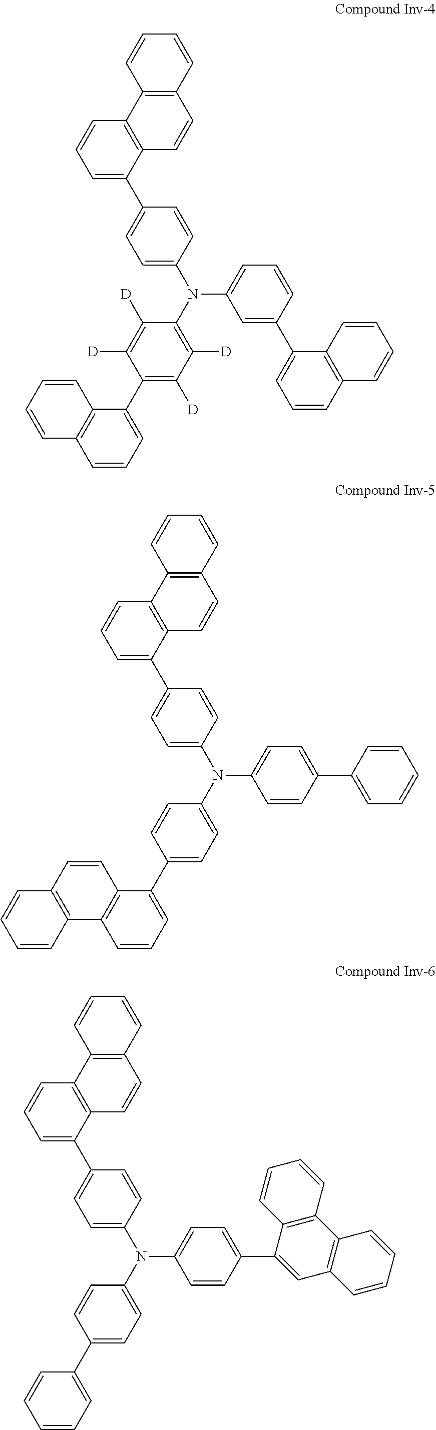 [SMILES: [2H]c1c([2H])c(N(c2ccc(-c3cccc4c3ccc3ccccc34)cc2)c2cccc(-c3cccc4ccccc34)c2)c([2H])c([2H])c1-c1cccc2ccccc12 ; c1ccc(-c2ccc(N(c3ccc(-c4cccc5c4ccc4ccccc45)cc3)c3ccc(-c4cccc5c4ccc4ccccc45)cc3)cc2)cc1 ; c1ccc(-c2ccc(N(c3ccc(-c4cccc5c4ccc4ccccc45)cc3)c3ccc(-c4cc5ccccc5c5ccccc45)cc3)cc2)cc1]

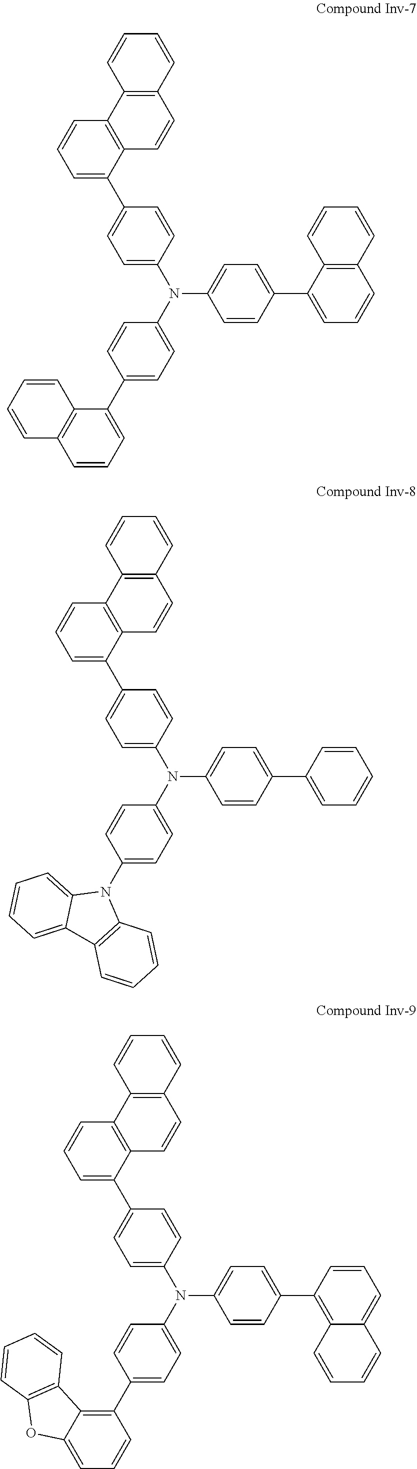 [SMILES: c1ccc2c(-c3ccc(N(c4ccc(-c5cccc6ccccc56)cc4)c4ccc(-c5cccc6c5ccc5ccccc56)cc4)cc3)cccc2c1 ; c1ccc(-c2ccc(N(c3ccc(-c4cccc5c4ccc4ccccc45)cc3)c3ccc(-n4c5ccccc5c5ccccc54)cc3)cc2)cc1 ; c1ccc2c(-c3ccc(N(c4ccc(-c5cccc6c5ccc5ccccc56)cc4)c4ccc(-c5cccc6oc7ccccc7c56)cc4)cc3)cccc2c1]

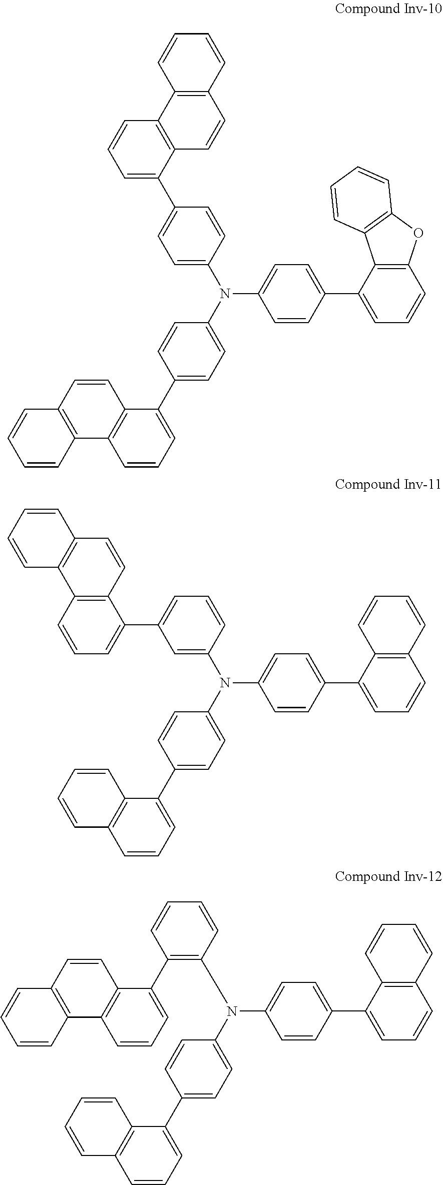

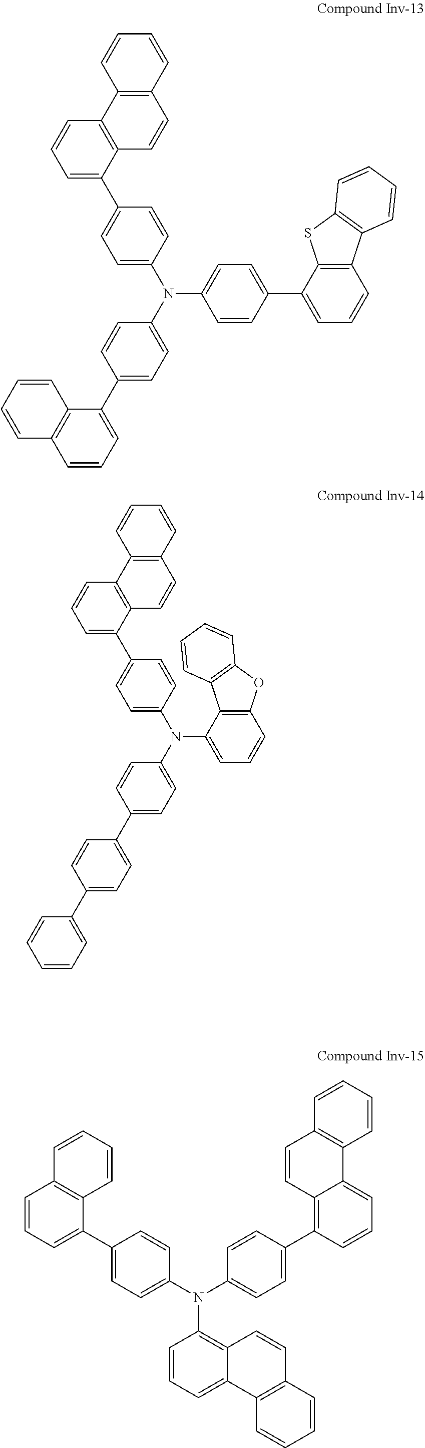 [SMILES: c1ccc2c(-c3ccc(N(c4ccc(-c5cccc6c5ccc5ccccc56)cc4)c4ccc(-c5cccc6c5sc5ccccc56)cc4)cc3)cccc2c1 ; c1ccc(-c2ccc(-c3ccc(N(c4ccc(-c5cccc6c5ccc5ccccc56)cc4)c4cccc5oc6ccccc6c45)cc3)cc2)cc1 ; c1ccc2c(-c3ccc(N(c4ccc(-c5cccc6c5ccc5ccccc56)cc4)c4cccc5c4ccc4ccccc45)cc3)cccc2c1]

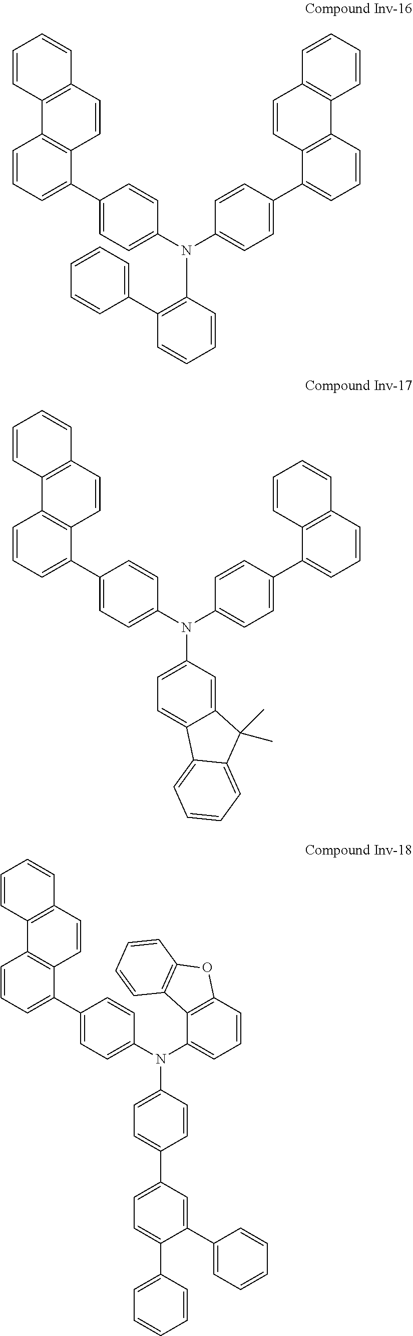 [SMILES: c1ccc(-c2ccccc2N(c2ccc(-c3cccc4c3ccc3ccccc34)cc2)c2ccc(-c3cccc4c3ccc3ccccc34)cc2)cc1 ; CC1(C)c2ccccc2-c2ccc(N(c3ccc(-c4cccc5ccccc45)cc3)c3ccc(-c4cccc5c4ccc4ccccc45)cc3)cc21 ; c1ccc(-c2ccc(-c3ccc(N(c4ccc(-c5cccc6c5ccc5ccccc56)cc4)c4cccc5oc6ccccc6c45)cc3)cc2-c2ccccc2)cc1]

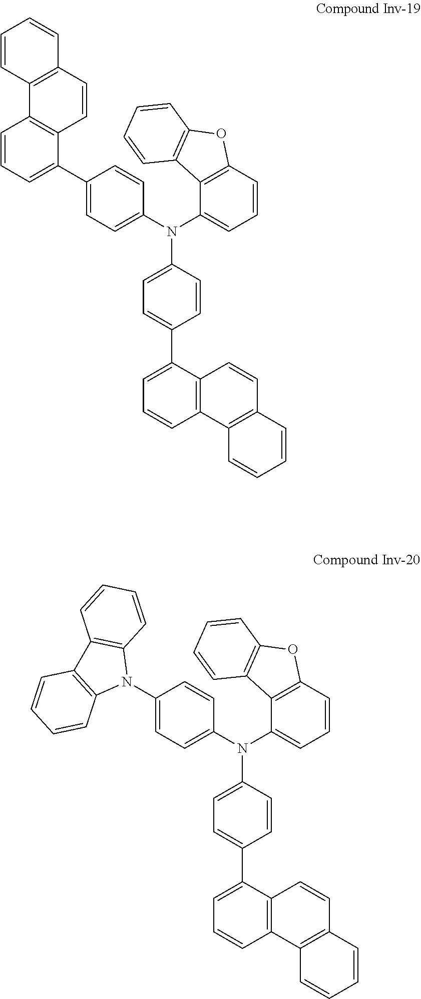 [SMILES: c1ccc2c(c1)ccc1c(-c3ccc(N(c4ccc(-c5cccc6c5ccc5ccccc56)cc4)c4cccc5oc6ccccc6c45)cc3)cccc12 ; c1ccc2c(c1)ccc1c(-c3ccc(N(c4ccc(-n5c6ccccc6c6ccccc65)cc4)c4cccc5oc6ccccc6c45)cc3)cccc12]

### Intermediate Synthesis Example 1: Synthesis of Intermediate A

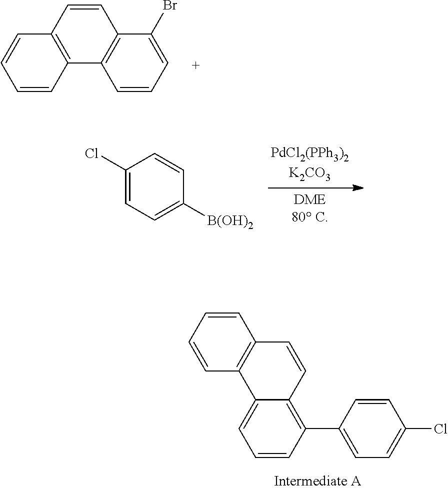 [SMILES: Brc1cccc2c1ccc1ccccc12 ; OB(O)c1ccc(Cl)cc1 ; Clc1ccc(-c2cccc3c2ccc2ccccc23)cc1]

Under an argon atmosphere, a mixture of 1-bromophenanthrene (3.7 g, 14.39 mmol), 4-chlorophenylboronic acid (2.70 g, 17.27 mmol), bis(triphenylphosphine)palladium(II) dichloride (0.303 g, 0.432 mmol), potassium carbonate (15.84 g, 43.2 mmol), DME (96 mL), and water (21.58 mL) synthesized in the same manner as the method described in “Synthesis, pp. 610-611 (1973)” was stirred at 80° C. for 7 hours. The reaction mixture was cooled to room temperature and concentrated under reduced pressure. The obtained residue was purified by silica gel column chromatography to obtain Intermediate A as a white solid (3.62 g). The yield was 87%.

### Intermediate Synthesis Example 2: Synthesis of Intermediate B

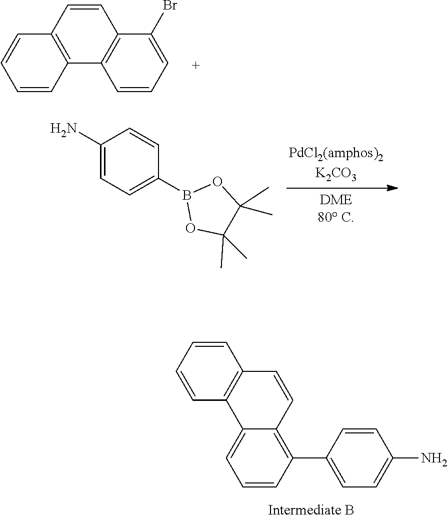

Under an argon atmosphere, a mixture of 1-bromophenanthrene (8.61 g, 33.5 mmol), 2-(4-aminophenyl)-4,4,5,5-tetramethyl-1,3,2-dioxaborolane (7.34 μg, 33.5 μmmol), dichlorobis[di-t-butyl(p-dimethylaminophenyl)phosphino]palladium(II) (0.712 g, 1.006 mmol), potassium carbonate (13.96 g, 101 mmol), DME (224 mL), and water (50.3 mL) was stirred at 80° C. for 7 hours. The reaction mixture was cooled to room temperature and concentrated under reduced pressure. The obtained residue was purified by silica gel column chromatography to obtain Intermediate B as alight yellow solid (6.47 g). The yield was 68%.

### Intermediate Synthesis Example 3: Synthesis of Intermediate C

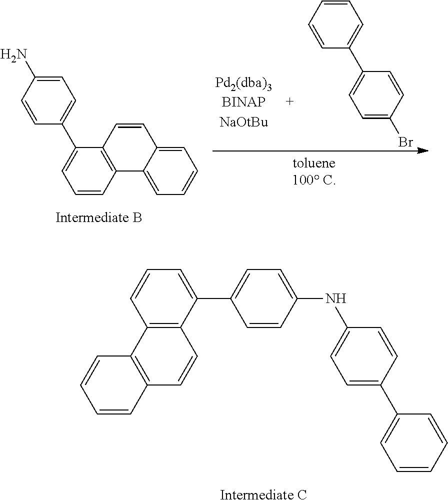 [SMILES: Nc1ccc(-c2cccc3c2ccc2ccccc23)cc1 ; Brc1ccc(-c2ccccc2)cc1 ; c1ccc(-c2ccc(Nc3ccc(-c4cccc5c4ccc4ccccc45)cc3)cc2)cc1]

Under an argon atmosphere, a mixture of Intermediate B (6.73 g, 25 mmol), 4-bromobiphenyl (5.83 g, 25 mmol), tris(dibenzylideneacetone)dipalladium(0) (0.229 g, 0.25 mmol), BINAP (0.311 g, 0.5 mmol), sodium-t-butoxide (3.36 g, 35 mmol), and toluene (167 mL) was stirred at 100° C. for 5 hours. The reaction mixture was cooled to room temperature and concentrated under reduced pressure. The obtained residue was purified by silica gel column chromatography to obtain Intermediate C as a white solid (7.00 g). The yield was 66%.

### Intermediate Synthesis Example 4: Synthesis of Intermediate D

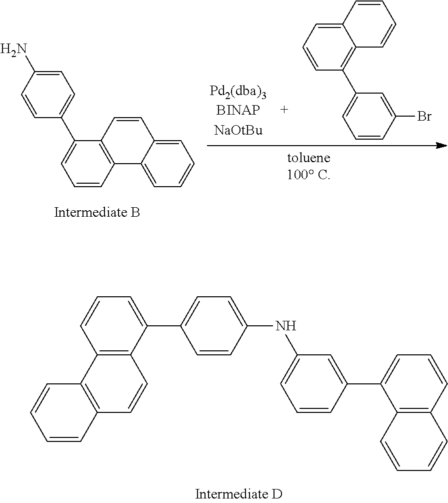 [SMILES: Nc1ccc(-c2cccc3c2ccc2ccccc23)cc1 ; Brc1cccc(-c2cccc3ccccc23)c1 ; c1cc(Nc2ccc(-c3cccc4c3ccc3ccccc34)cc2)cc(-c2cccc3ccccc23)c1]

Under an argon atmosphere, a mixture of Intermediate B (6.4 g, 23.76 mmol), 1-bromo-3-(1-naphthyl) benzene (6.37 g, 23.76 mmol), tris(dibenzylideneacetone)dipalladium(0) (0.218 g, 0.238 mmol), BINAP (0.296 g, 0.475 mmol), sodium-t-butoxide (3.20 g, 33.3 mmol), and toluene (158 mL) was stirred at 100° C. for 5 hours. The reaction mixture was cooled to room temperature and concentrated under reduced pressure. The obtained residue was purified by silica gel column chromatography to obtain Intermediate D as a white solid (7.98 g). The yield was 71%.

### Intermediate Synthesis Example 5: Synthesis of Intermediate E

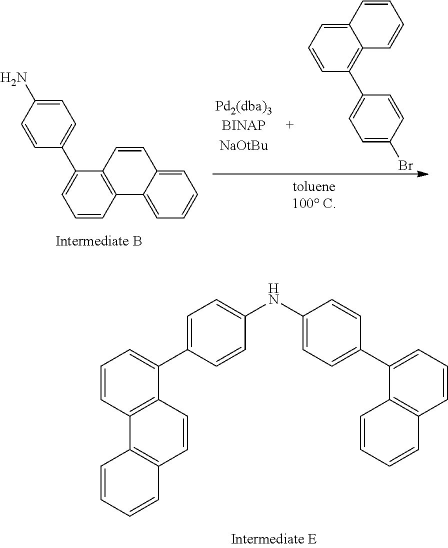 [SMILES: Nc1ccc(-c2cccc3c2ccc2ccccc23)cc1 ; Brc1ccc(-c2cccc3ccccc23)cc1 ; c1ccc2c(-c3ccc(Nc4ccc(-c5cccc6c5ccc5ccccc56)cc4)cc3)cccc2c1]

Under an argon atmosphere, a mixture of Intermediate B (6.4 g, 23.76 mmol), 1-bromo-4-(1-naphthyl) benzene (6.37 g, 23.76 mmol), tris(dibenzylideneacetone)dipalladium(0) (0.218 g, 0.238 mmol), BINAP (0.296 g, 0.475 mmol), sodium-t-butoxide (3.20 g, 33.3 mmol), and toluene (158 mL) was stirred at 100° C. for 3 hours. The reaction mixture was cooled to room temperature and concentrated under reduced pressure. The obtained residue was purified by silica gel column chromatography to obtain Intermediate E as a white solid (8.99 g). The yield was 80%.

### Intermediate Synthesis Example 6: Synthesis of Intermediate F

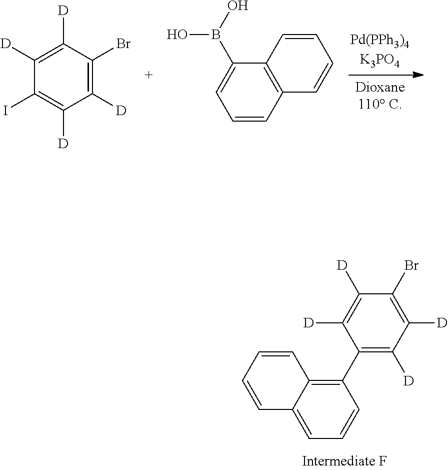 [SMILES: [2H]c1c([2H])c(I)c([2H])c([2H])c1Br ; OB(O)c1cccc2ccccc12 ; [2H]c1c([2H])c(-c2cccc3ccccc23)c([2H])c([2H])c1Br]

Under an argon atmosphere, a mixture of 1-bromo-4-iodobenzene-2,3,5,6-d4 (2.87 g, 10 μmmol), 1-naphthalene boronic acid (1.806 μg, 10.5 μmmol), tetrakis(triphenylphosphine)palladium(0) (0.462 g, 0.400 mmol), tripotassium phosphate (4.245 g, 20 mmol), and 1,4-dioxane (158 mL) synthesized in the same manner as the method described in WO 2011/040939 was stirred at 110° C. for 5 hours. The reaction mixture was cooled to room temperature and concentrated under reduced pressure. The obtained residue was purified by silica gel column chromatography to obtain Intermediate F as a white solid (2.87 g). The yield was 100%.

### Intermediate Synthesis Example 7: Synthesis of Intermediate G

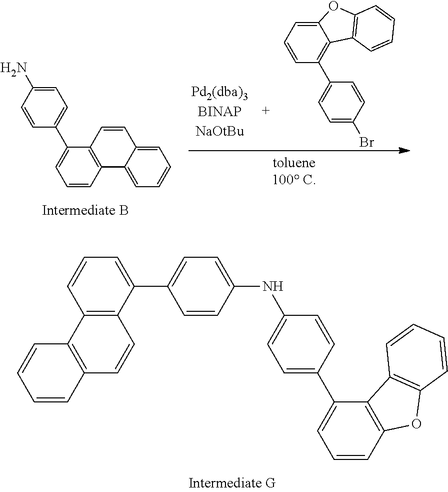 [SMILES: Nc1ccc(-c2cccc3c2ccc2ccccc23)cc1 ; Brc1ccc(-c2cccc3oc4ccccc4c23)cc1 ; c1ccc2c(c1)ccc1c(-c3ccc(Nc4ccc(-c5cccc6oc7ccccc7c56)cc4)cc3)cccc12]

Under an argon atmosphere, a mixture of Intermediate B (4 g, 14.85 mmol), 1-(4-bromophenyl)dibenzo[b,d]furan (4.80 μg, 14.85 μmmol), tris(dibenzylideneacetone)dipalladium(0) (0.272 g, 0.297 mmol), BINAP (0.594 g, 0.370 mmol), sodium-t-butoxide (3.36 g, 35 mmol), and toluene (100 mL) was stirred at 100° C. for 5 hours. The reaction mixture was cooled to room temperature and concentrated under reduced pressure. The obtained residue was purified by silica gel column chromatography to obtain Intermediate G as a white solid (5.66 g). The yield was 75%.

### Intermediate Synthesis Example 8: Synthesis of Intermediate H

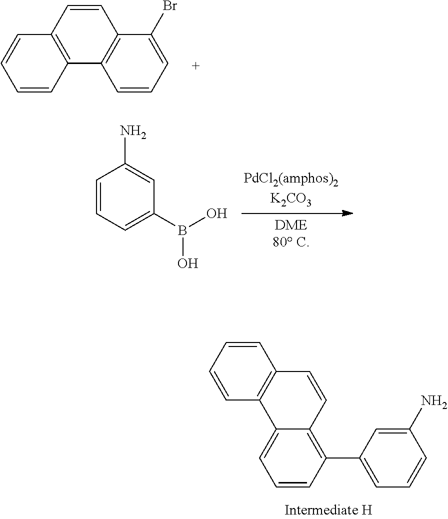

Under an argon atmosphere, a mixture of 1-bromophenanthrene (3.4 g, 13.22 mmol), 3-aminophenylboronic acid monohydrate (2.05 g, 13.22 mmol), dichlorobis[di-t-butyl(p-dimethylaminophenyl)phosphino]palladium(II) (0.187 g, 0.264 mmol), potassium carbonate (5.47 g, 39.7 mmol), DME (66 mL), and water (19.8 mL) was stirred at 80° C. for 7 hours. The reaction mixture was cooled to room temperature and concentrated under reduced pressure. The obtained residue was purified by silica gel column chromatography to obtain Intermediate H as a light yellow solid (2.17 g). The yield was 61%.

### Intermediate Synthesis Example 9: Synthesis of Intermediate I

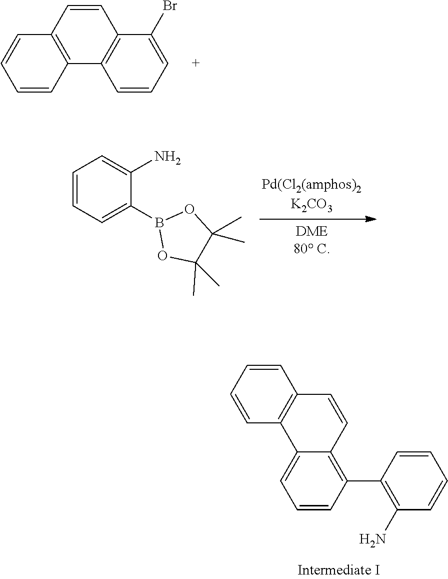 [SMILES: Brc1cccc2c1ccc1ccccc12 ; CC1(C)OB(c2ccccc2N)OC1(C)C ; Nc1ccccc1-c1cccc2c1ccc1ccccc12]

Under an argon atmosphere, a mixture of 1-bromophenanthrene (3.4 g, 13.22 mmol), 2-(2-aminophenyl)-4,4,5,5-tetramethyl-1,3,2-dioxaborolane (2.90 μg, 13.22 μmmol), dichlorobis[di-t-butyl(p-dimethylaminophenyl)phosphino]palladium(II) (0.187 g, 0.264 mmol), potassium carbonate (5.48 g, 39.7 mmol), DME (66 mL), and water (19.8 mL) was stirred at 80° C. for 5 hours. The reaction mixture was cooled to room temperature and concentrated under reduced pressure. The obtained residue was purified by silica gel column chromatography to obtain Intermediate I as a light yellow solid (3.04 g). The yield was 85%.

### Intermediate Synthesis Example 10: Synthesis of Intermediate J

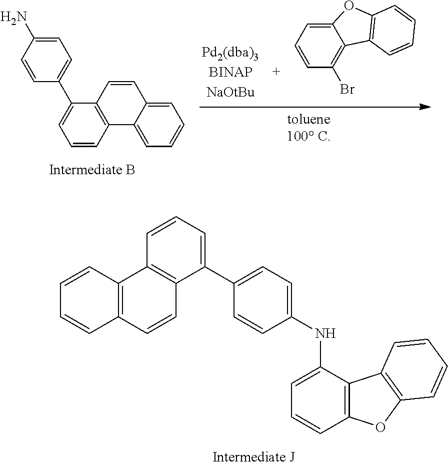 [SMILES: Nc1ccc(-c2cccc3c2ccc2ccccc23)cc1 ; Brc1cccc2oc3ccccc3c12 ; c1ccc2c(c1)ccc1c(-c3ccc(Nc4cccc5oc6ccccc6c45)cc3)cccc12]

Under an argon atmosphere, a mixture of Intermediate B (10 g, 37.1 mmol), 1-bromodibenzo[b,d]furan (9.17 g, 37.1 mmol), tris(dibenzylideneacetone)dipalladium(0) (0.680 g, 0.743 mmol), BINAP (0.925 g, 1.485 mmol), sodium-t-butoxide (7.85 g, 82 mmol), and toluene (186 mL) was stirred at 100° C. for 5 hours. The reaction mixture was cooled to room temperature and concentrated under reduced pressure. The obtained residue was purified by silica gel column chromatography to obtain Intermediate J as a white solid (15.2 g). The yield was 94%.

### Synthesis Example 1: Synthesis of Compound Inv-1

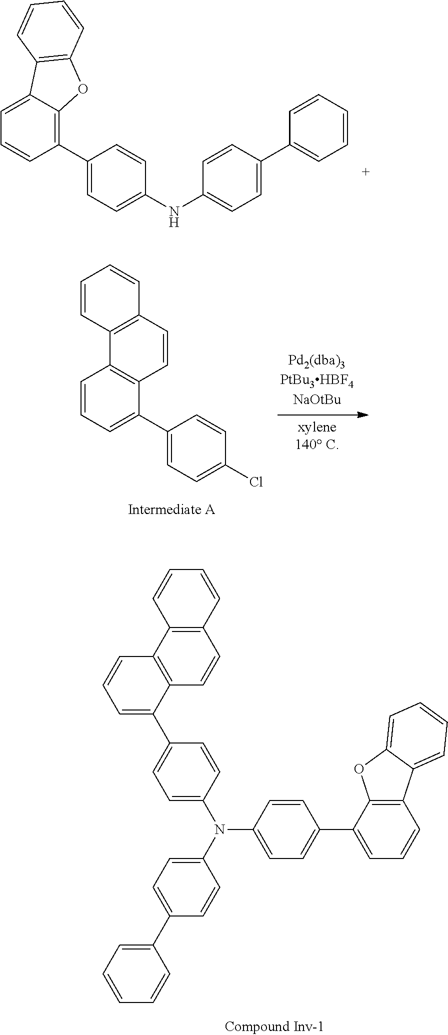 [SMILES: c1ccc(-c2ccc(Nc3ccc(-c4cccc5c4oc4ccccc45)cc3)cc2)cc1 ; Clc1ccc(-c2cccc3c2ccc2ccccc23)cc1 ; c1ccc(-c2ccc(N(c3ccc(-c4cccc5c4ccc4ccccc45)cc3)c3ccc(-c4cccc5c4oc4ccccc45)cc3)cc2)cc1]

Under an argon atmosphere, a mixture of N-(4-(dibenzo[b,d]furan-4-yl)phenyl)-[1,1′-biphenyl]-4-amine (3.43 g, 8.34 mmol), Intermediate A (2.53 g, 8.75 mmol), tris(dibenzylideneacetone)dipalladium(0) (0.153 g, 0.167 mmol), tri-t-butylphosphonium tetrafluoroborate (0.193 g, 0.667 mmol), sodium-t-butoxide (1.121 g, 11.67 mmol), and xylene (56 mL) synthesized in the same manner as the method described in WO 2014/132636 was stirred at 140° C. for 3 hours. The reaction mixture was cooled to room temperature and concentrated under reduced pressure. The obtained residue was purified by silica gel column chromatography and recrystallization to obtain 3.65 g of a white solid. The yield was 66%. As a result of mass spectrum analysis, the obtained product was Compound Inv-1, and m/e was 663 with respect to the molecular weight of 663.26.

### Synthesis Example 2: Synthesis of Compound Inv-2

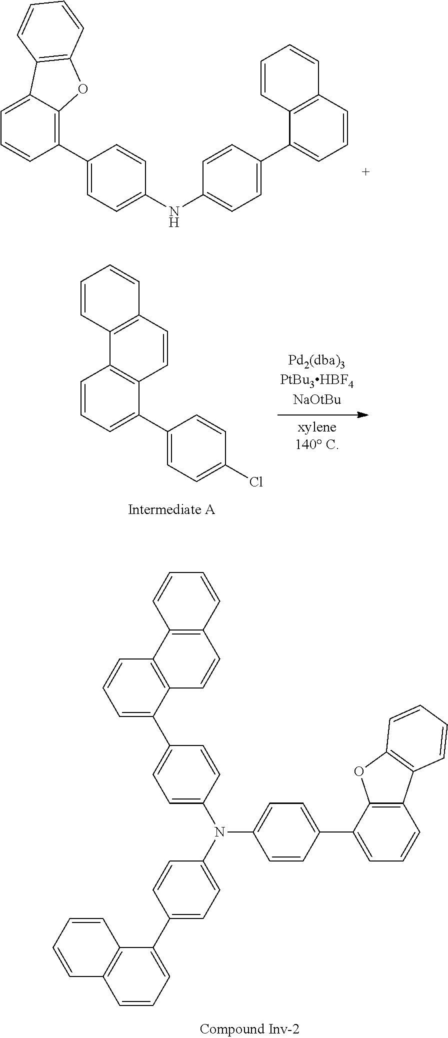 [SMILES: c1ccc2c(-c3ccc(Nc4ccc(-c5cccc6c5oc5ccccc56)cc4)cc3)cccc2c1 ; Clc1ccc(-c2cccc3c2ccc2ccccc23)cc1 ; c1ccc2c(-c3ccc(N(c4ccc(-c5cccc6c5ccc5ccccc56)cc4)c4ccc(-c5cccc6c5oc5ccccc56)cc4)cc3)cccc2c1]

The same operation as in Synthesis Example 1 was performed except that 4-(dibenzo[b,d]furan-4-yl)-N-(4-(naphthalen-1-yl)phenyl)aniline synthesized in the same manner as in the method described in WO 2019/146781 was used instead of N-(4-(dibenzo[b,d]furan-4-yl)phenyl)-[1,1′-biphenyl]-4-amine used in Synthesis Example 1 to obtain a white solid of 7.64 g. The yield was 79%.

As a result of mass spectrum analysis, the obtained product was Compound Inv-2, and m/e was 713 with respect to the molecular weight of 713.27.

### Synthesis Example 3: Synthesis of Compound Inv-3

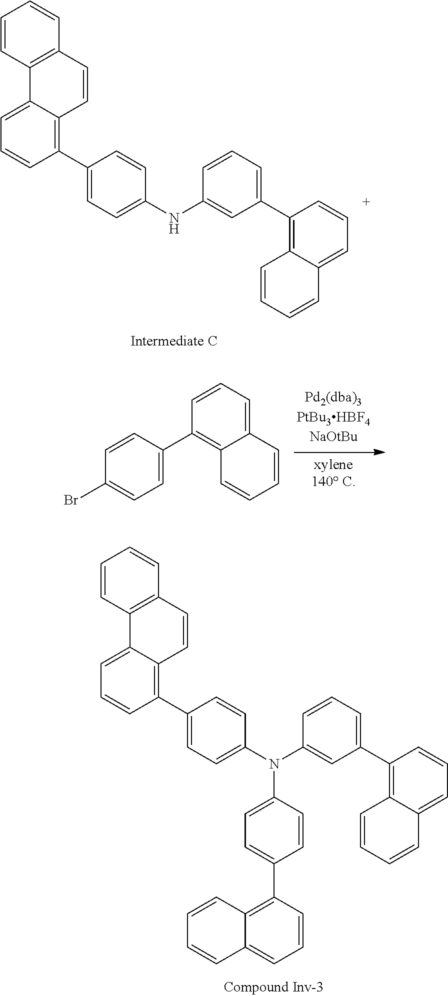 [SMILES: c1cc(Nc2ccc(-c3cccc4c3ccc3ccccc34)cc2)cc(-c2cccc3ccccc23)c1 ; Brc1ccc(-c2cccc3ccccc23)cc1 ; c1cc(-c2cccc3ccccc23)cc(N(c2ccc(-c3cccc4ccccc34)cc2)c2ccc(-c3cccc4c3ccc3ccccc34)cc2)c1]

The same operation as in Synthesis Example 1 was performed except that Intermediate C was used instead of N-(4-(dibenzo[b,d]furan-4-yl)phenyl)-[1,1′-biphenyl]-4-amine used in Synthesis Example 1, and 1-(4-bromophenyl)naphthalene was used instead of Intermediate A used in Synthesis Example 1 to obtain a white solid of 5.01 g. The yield was 99%.

As a result of mass spectrum analysis, the obtained product was Compound Inv-3, and m/e was 673 with respect to the molecular weight of 673.28.

### Synthesis Example 4: Synthesis of Compound Inv-4

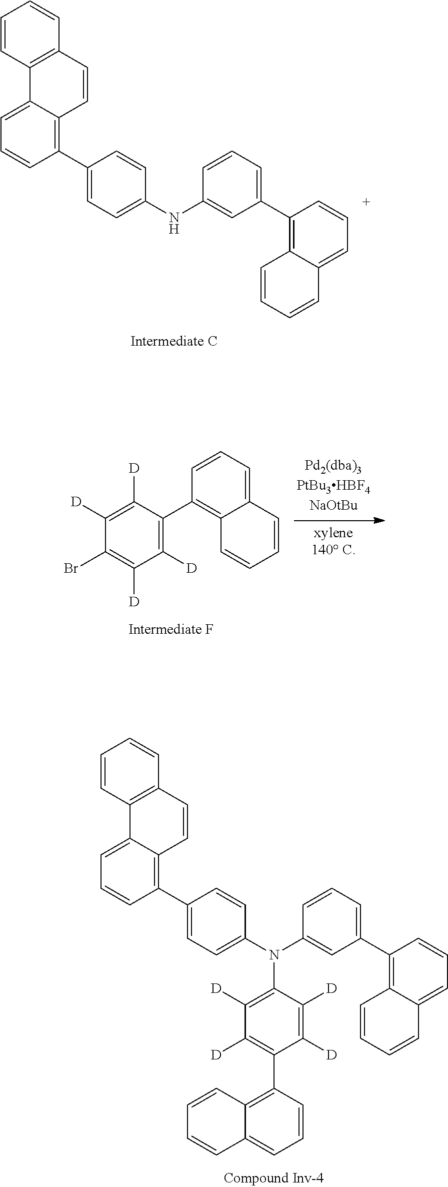 [SMILES: c1cc(Nc2ccc(-c3cccc4c3ccc3ccccc34)cc2)cc(-c2cccc3ccccc23)c1 ; [2H]c1c([2H])c(-c2cccc3ccccc23)c([2H])c([2H])c1Br ; [2H]c1c([2H])c(N(c2ccc(-c3cccc4c3ccc3ccccc34)cc2)c2cccc(-c3cccc4ccccc34)c2)c([2H])c([2H])c1-c1cccc2ccccc12]

The same operation as in Synthesis Example 1 was performed except that Intermediate C was used instead of N-(4-(dibenzo[b,d]furan-4-yl)phenyl)-[1,1′-biphenyl]-4-amine used in Synthesis Example 1, and Intermediate F was used instead of Intermediate A used in Synthesis Example 1 to obtain a white solid of 4.32 g. The yield was 88%.

As a result of mass spectrum analysis, the obtained product was Compound Inv-4, and m/e was 677 with respect to the molecular weight of 677.30.

### Synthesis Example 5: Synthesis of Compound Inv-5

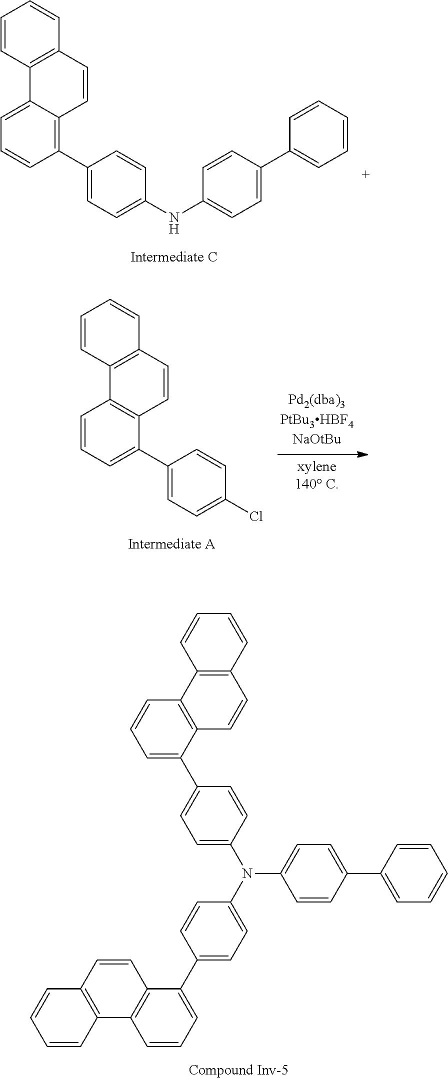 [SMILES: c1ccc(-c2ccc(Nc3ccc(-c4cccc5c4ccc4ccccc45)cc3)cc2)cc1 ; Clc1ccc(-c2cccc3c2ccc2ccccc23)cc1 ; c1ccc(-c2ccc(N(c3ccc(-c4cccc5c4ccc4ccccc45)cc3)c3ccc(-c4cccc5c4ccc4ccccc45)cc3)cc2)cc1]

The same operation as in Synthesis Example 1 was performed except that Intermediate C was used instead of N-(4-(dibenzo[b,d]furan-4-yl)phenyl)-[1,1′-biphenyl]-4-amine used in Synthesis Example 1 to obtain a white solid of 4.96 g. The yield was 99%.

As a result of mass spectrum analysis, the obtained product was Compound Inv-5, and m/e was 673 with respect to the molecular weight of 673.28.

### Synthesis Example 6: Synthesis of Compound Inv-6

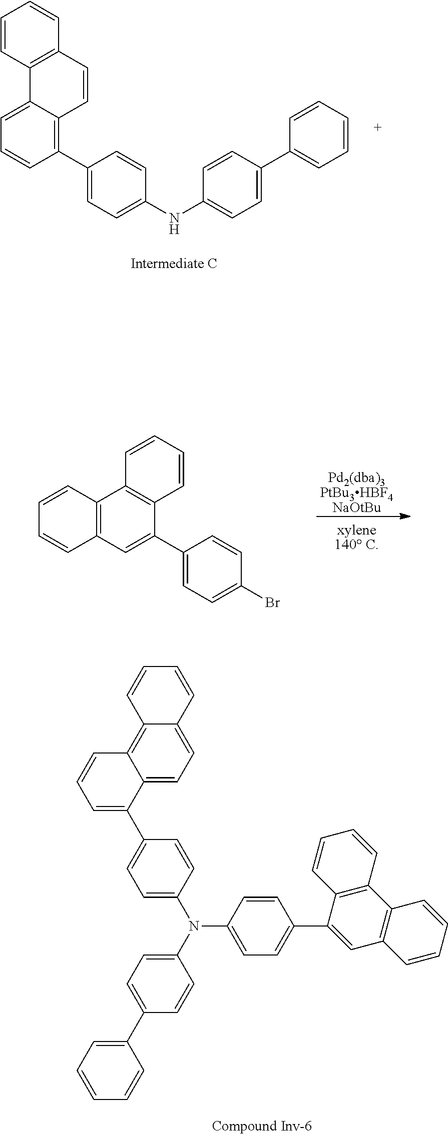 [SMILES: c1ccc(-c2ccc(Nc3ccc(-c4cccc5c4ccc4ccccc45)cc3)cc2)cc1 ; Brc1ccc(-c2cc3ccccc3c3ccccc23)cc1 ; "Pd2(dba)3PtBu3·HBF4NaOtBu" ; "xylene140° C." ; c1ccc(-c2ccc(N(c3ccc(-c4cccc5c4ccc4ccccc45)cc3)c3ccc(-c4cc5ccccc5c5ccccc45)cc3)cc2)cc1]

The same operation as in Synthesis Example 1 was performed except that Intermediate C was used instead of N-(4-(dibenzo[b,d]furan-4-yl)phenyl)-[1,1′-biphenyl]-4-amine used in Synthesis Example 1, and 9-(4-bromophenyl)phenanthrene synthesized in the same manner as in the method described in WO 2013/062075 was used instead of Intermediate A used in Synthesis Example 1 to obtain a white solid of 4.83 g. The yield was 75%.

As a result of mass spectrum analysis, the obtained product was Compound Inv-6, and m/e was 673 with respect to the molecular weight of 673.28.

### Synthesis Example 7: Synthesis of Compound Inv-7

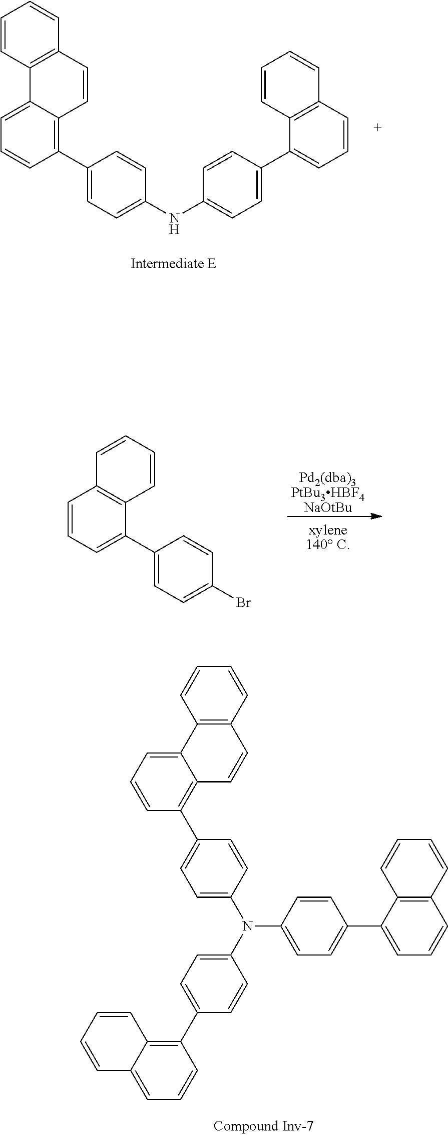 [SMILES: c1ccc2c(-c3ccc(Nc4ccc(-c5cccc6c5ccc5ccccc56)cc4)cc3)cccc2c1 ; Brc1ccc(-c2cccc3ccccc23)cc1 ; "Pd2(dba)3PtBu3·HBF4NaOtBu" ; "xylene140° C." ; c1ccc2c(-c3ccc(N(c4ccc(-c5cccc6ccccc56)cc4)c4ccc(-c5cccc6c5ccc5ccccc56)cc4)cc3)cccc2c1]

The same operation as in Synthesis Example 1 was performed except that Intermediate E was used instead of N-(4-(dibenzo[b,d]furan-4-yl)phenyl)-[1,1′-biphenyl]-4-amine used in Synthesis Example 1, and 1-(4-bromophenyl)naphthalene was used instead of Intermediate A used in Synthesis Example 1 to obtain a white solid of 4.99 g. The yield was 78%.

As a result of mass spectrum analysis, the obtained product was Compound Inv-7, and m/e was 673 with respect to the molecular weight of 673.28.

### Synthesis Example 8: Synthesis of Compound Inv-8

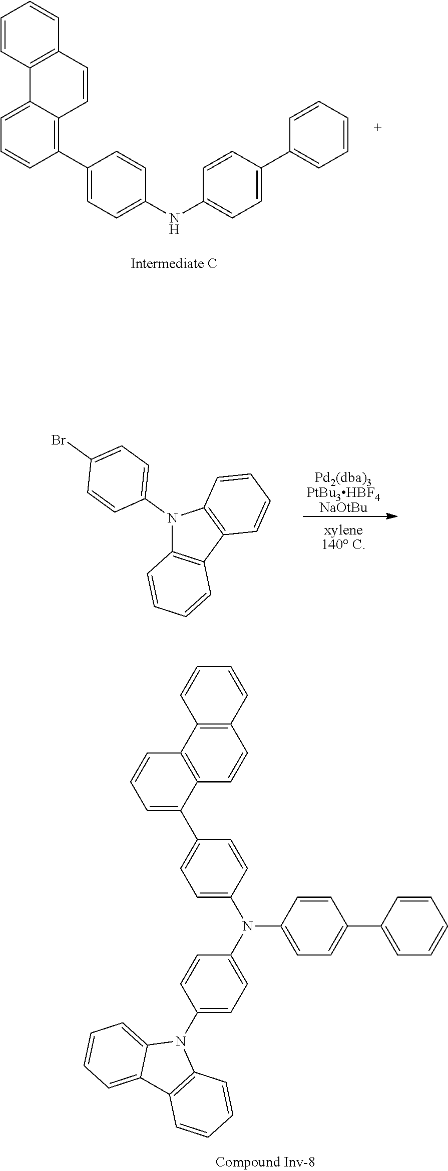 [SMILES: c1ccc(-c2ccc(Nc3ccc(-c4cccc5c4ccc4ccccc45)cc3)cc2)cc1 ; Brc1ccc(-n2c3ccccc3c3ccccc32)cc1 ; "Pd2(dba)3PtBu3·HBF4NaOtBu" ; "xylene140° C." ; c1ccc(-c2ccc(N(c3ccc(-c4cccc5c4ccc4ccccc45)cc3)c3ccc(-n4c5ccccc5c5ccccc54)cc3)cc2)cc1]

The same operation as in Synthesis Example 1 was performed except that Intermediate C was used instead of N-(4-(dibenzo[b,d]furan-4-yl)phenyl)-[1,1′-biphenyl]-4-amine used in Synthesis Example 1, and 9-(4-bromophenyl)-9H-carbazole synthesized in the same manner as in the method described in JP 2004-217557 A was used instead of Intermediate A used in Synthesis Example 1 to obtain a white solid of 5.47 g. The yield was 87%.

As a result of mass spectrum analysis, the obtained product was Compound Inv-8, and m/e was 662 with respect to the molecular weight of 662.27.

### Synthesis Example 9: Synthesis of Compound Inv-9

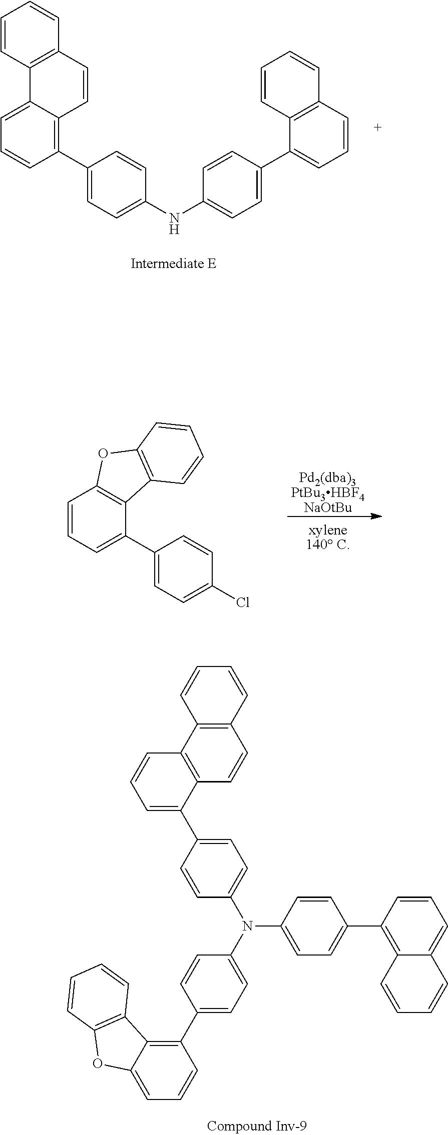 [SMILES: c1ccc2c(-c3ccc(Nc4ccc(-c5cccc6c5ccc5ccccc56)cc4)cc3)cccc2c1 ; Clc1ccc(-c2cccc3oc4ccccc4c23)cc1 ; "Pd2(dba)3PtBu3·HBF4NaOtBu" ; "xylene140° C." ; c1ccc2c(-c3ccc(N(c4ccc(-c5cccc6c5ccc5ccccc56)cc4)c4ccc(-c5cccc6oc7ccccc7c56)cc4)cc3)cccc2c1]

The same operation as in Synthesis Example 1 was performed except that Intermediate E was used instead of N-(4-(dibenzo[b,d]furan-4-yl)phenyl)-[1,1′-biphenyl]-4-amine used in Synthesis Example 1, and 1-(4-chlorophenyl)dibenzo[b,d]furan synthesized in the same manner as in the method described in WO 2018/164201 was used instead of Intermediate A used in Synthesis Example 1 to obtain a white solid of 5.84 g. The yield was 82%.

As a result of mass spectrum analysis, the obtained product was Compound Inv-9, and m/e was 713 with respect to the molecular weight of 713.27.

### Synthesis Example 10: Synthesis of Compound Inv-10

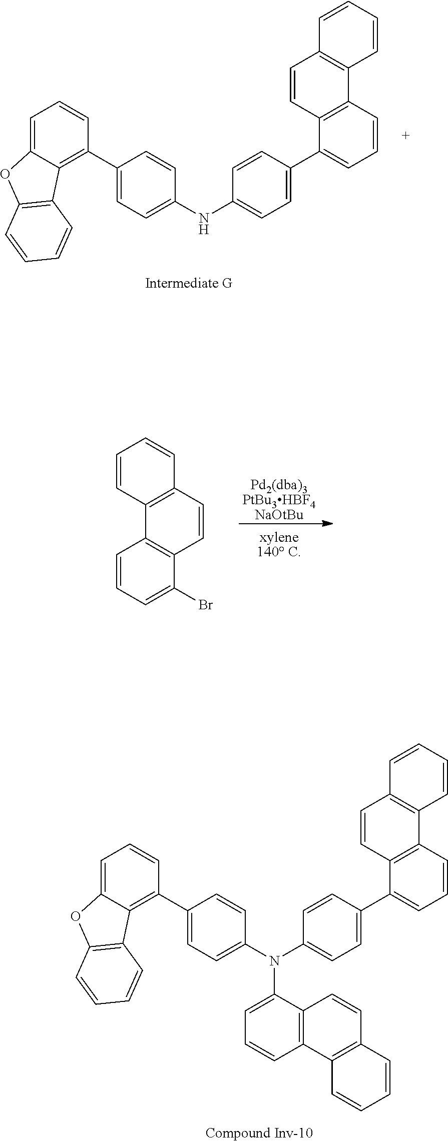 [SMILES: c1ccc2c(c1)ccc1c(-c3ccc(Nc4ccc(-c5cccc6oc7ccccc7c56)cc4)cc3)cccc12 ; Brc1cccc2c1ccc1ccccc12 ; "Pd2(dba)3PtBu3·HBF4NaOtBu" ; "xylene140° C." ; c1ccc2c(c1)ccc1c(-c3ccc(N(c4ccc(-c5cccc6oc7ccccc7c56)cc4)c4cccc5c4ccc4ccccc45)cc3)cccc12]

The same operation as in Synthesis Example 1 was performed except that Intermediate G was used instead of N-(4-(dibenzo[b,d]furan-4-yl)phenyl)-[1,1′-biphenyl]-4-amine used in Synthesis Example 1, and 1-bromophenanthrene was used instead of Intermediate A used in Synthesis Example 1 to obtain a solid of 4.73 g. The yield was 78%.

As a result of mass spectrum analysis, the obtained product was Compound Inv-10, and m/e was 687 with respect to the molecular weight of 687.26.

### Synthesis Example 11: Synthesis of Compound Inv-11

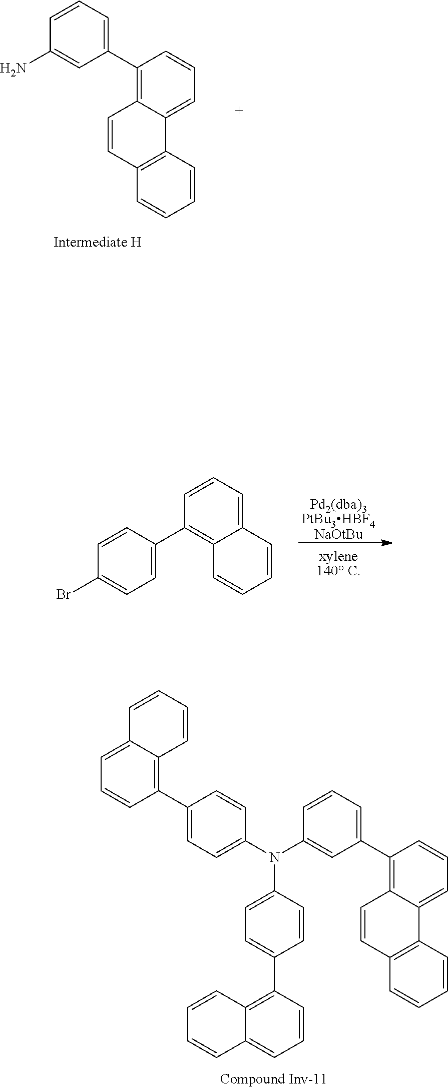 [SMILES: Nc1cccc(-c2cccc3c2ccc2ccccc23)c1 ; Brc1ccc(-c2cccc3ccccc23)cc1 ; "Pd2(dba)3PtBu3·HBF4NaOtBu" ; "xylene140° C." ; c1cc(-c2cccc3c2ccc2ccccc23)cc(N(c2ccc(-c3cccc4ccccc34)cc2)c2ccc(-c3cccc4ccccc34)cc2)c1]

The same operation as in Synthesis Example 1 was performed except that Intermediate H was used instead of N-(4-(dibenzo[b,d]furan-4-yl)phenyl)-[1,1′-biphenyl]-4-amine used in Synthesis Example 1, and 1-(4-bromophenyl)naphthalene was used instead of Intermediate A used in Synthesis Example 1 to obtain a solid of 3.46 g. The yield was 65%.

As a result of mass spectrum analysis, the obtained product was Compound Inv-11, and m/e was 673 with respect to the molecular weight of 673.28.

### Synthesis Example 12: Synthesis of Compound Inv-12

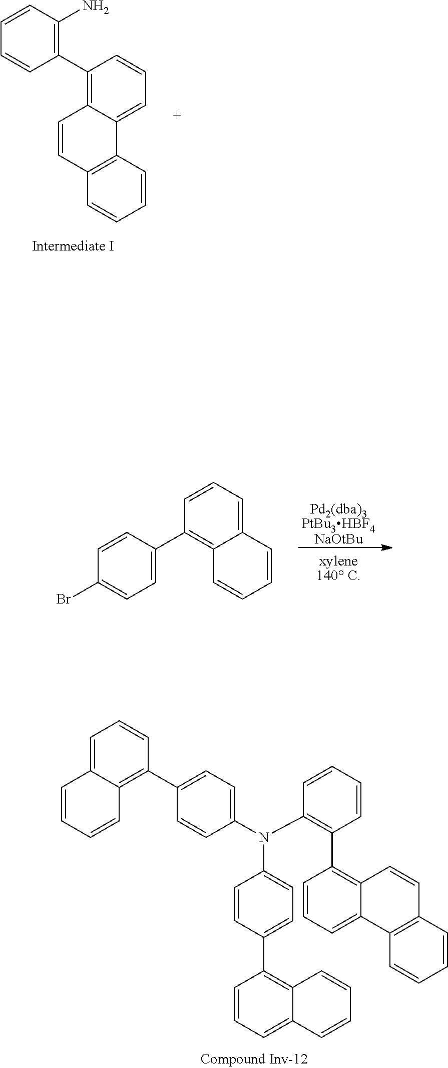 [SMILES: Nc1ccccc1-c1cccc2c1ccc1ccccc12 ; Brc1ccc(-c2cccc3ccccc23)cc1 ; "Pd2(dba)3PtBu3·HBF4NaOtBu" ; "xylene140° C." ; c1ccc(N(c2ccc(-c3cccc4ccccc34)cc2)c2ccc(-c3cccc4ccccc34)cc2)c(-c2cccc3c2ccc2ccccc23)c1]

The same operation as in Synthesis Example 1 was performed except that Intermediate I was used instead of N-(4-(dibenzo[b,d]furan-4-yl)phenyl)-[1,1′-biphenyl]-4-amine used in Synthesis Example 1, and 1-(4-bromophenyl)naphthalene was used instead of Intermediate A used in Synthesis Example 1 to obtain a solid of 4.16 g. The yield was 69%.

As a result of mass spectrum analysis, the obtained product was Compound Inv-12, and m/e was 673 with respect to the molecular weight of 673.28.

### Synthesis Example 13: Synthesis of Compound Inv-13

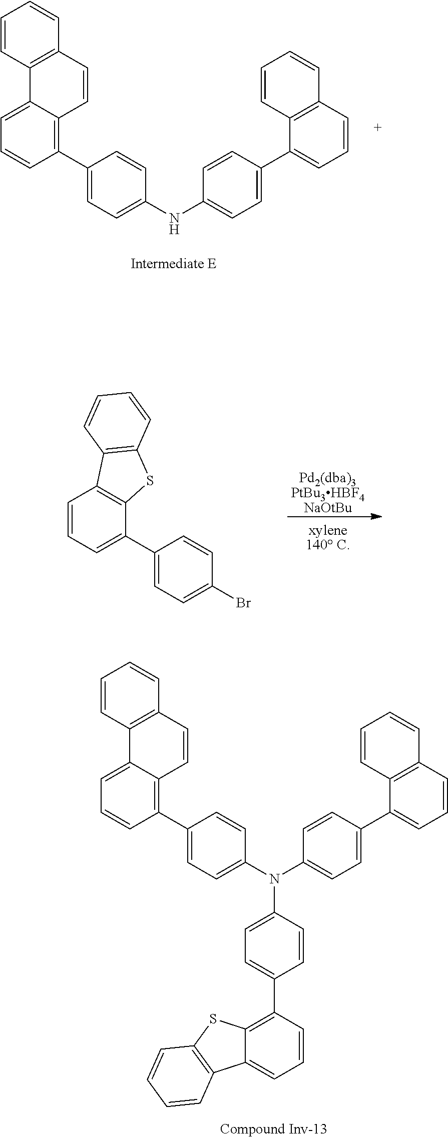 [SMILES: c1ccc2c(-c3ccc(Nc4ccc(-c5cccc6c5ccc5ccccc56)cc4)cc3)cccc2c1 ; Brc1ccc(-c2cccc3c2sc2ccccc23)cc1 ; "Pd2(dba)3PtBu3·HBF4NaOtBu" ; "xylene140° C." ; c1ccc2c(-c3ccc(N(c4ccc(-c5cccc6c5ccc5ccccc56)cc4)c4ccc(-c5cccc6c5sc5ccccc56)cc4)cc3)cccc2c1]

The same operation as in Synthesis Example 1 was performed except that Intermediate E was used instead of N-(4-(dibenzo[b,d]furan-4-yl)phenyl)-[1,1′-biphenyl]-4-amine used in Synthesis Example 1, and 4-(4-bromophenyl)dibenzothiophene was used instead of Intermediate A used in Synthesis Example 1 to obtain a solid of 5.56 g. The yield was 96%.

As a result of mass spectrum analysis, the obtained product was Compound Inv-13, and m/e was 729 with respect to the molecular weight of 729.25.

### Synthesis Example 14: Synthesis of Compound Inv-14

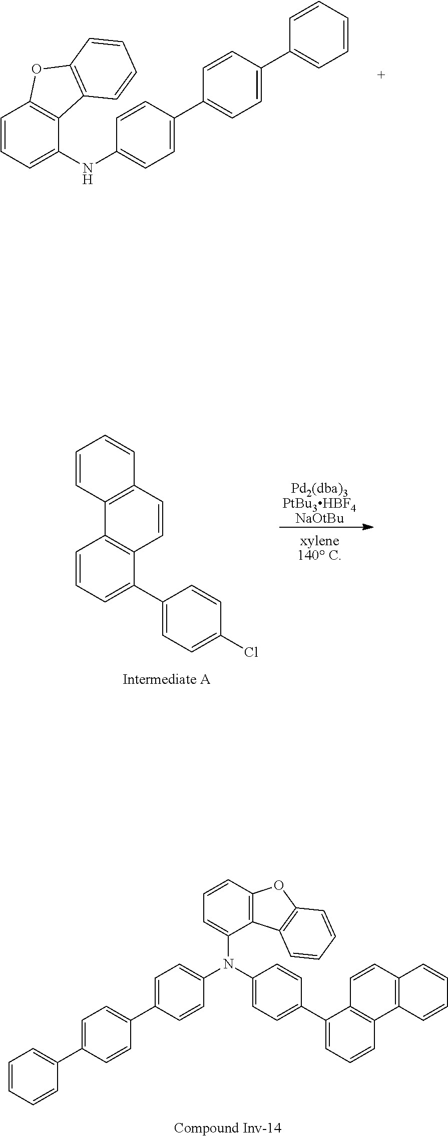 [SMILES: c1ccc(-c2ccc(-c3ccc(Nc4cccc5oc6ccccc6c45)cc3)cc2)cc1 ; Clc1ccc(-c2cccc3c2ccc2ccccc23)cc1 ; "Pd2(dba)3PtBu3·HBF4NaOtBu" ; "xylene140° C." ; c1ccc(-c2ccc(-c3ccc(N(c4ccc(-c5cccc6c5ccc5ccccc56)cc4)c4cccc5oc6ccccc6c45)cc3)cc2)cc1]

The same operation as in Synthesis Example 1 was performed except that N-([1,1′:4′,1″-terphenyl]-4-yl)dibenzo[b,d]furan-1-amine synthesized in the same manner as in the method described in WO 2020/111251 was used instead of N-(4-(dibenzo[b,d]furan-4-yl)phenyl)-[1,1′-biphenyl]-4-amine used in Synthesis Example 1 to obtain a solid of 3.86 g. The yield was 90%.

As a result of mass spectrum analysis, the obtained product was Compound Inv-14, and m/e was 663 with respect to the molecular weight of 663.26.

### Synthesis Example 15: Synthesis of Compound Inv-15

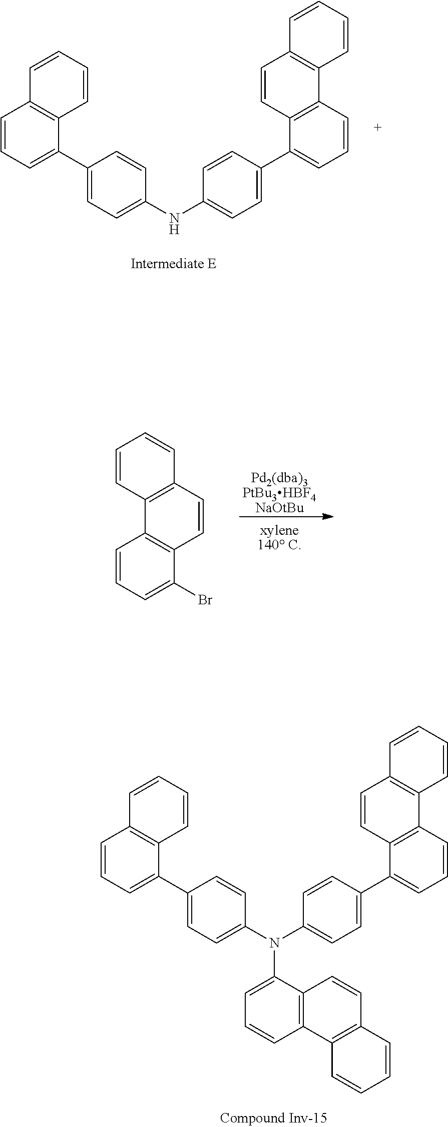 [SMILES: c1ccc2c(-c3ccc(Nc4ccc(-c5cccc6c5ccc5ccccc56)cc4)cc3)cccc2c1 ; Brc1cccc2c1ccc1ccccc12 ; "Pd2(dba)3PtBu3·HBF4NaOtBu" ; "xylene140° C." ; c1ccc2c(-c3ccc(N(c4ccc(-c5cccc6c5ccc5ccccc56)cc4)c4cccc5c4ccc4ccccc45)cc3)cccc2c1]

The same operation as in Synthesis Example 1 was performed except that Intermediate E was used instead of N-(4-(dibenzo[b,d]furan-4-yl)phenyl)-[1,1′-biphenyl]-4-amine used in Synthesis Example 1, and 1-bromophenanthrene was used instead of Intermediate A used in Synthesis Example 1 to obtain a solid of 4.21 g. The yield was 77%.

As a result of mass spectrum analysis, the obtained product was Compound Inv-15, and m/e was 647 with respect to the molecular weight of 647.26.

### Synthesis Example 16: Synthesis of Compound Inv-16

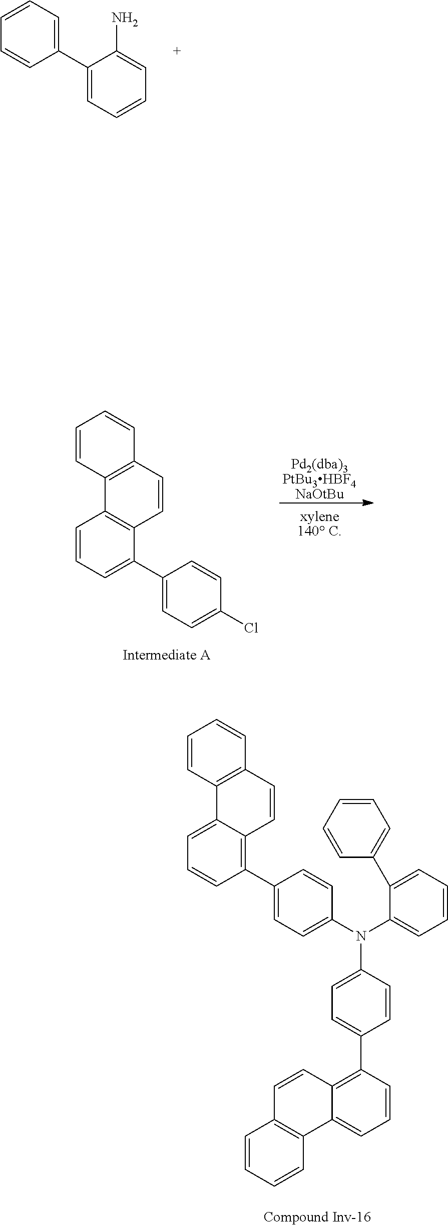 [SMILES: Nc1ccccc1-c1ccccc1 ; Clc1ccc(-c2cccc3c2ccc2ccccc23)cc1 ; "Pd2(dba)3PtBu3·HBF4NaOtBu" ; "xylene140° C." ; c1ccc(-c2ccccc2N(c2ccc(-c3cccc4c3ccc3ccccc34)cc2)c2ccc(-c3cccc4c3ccc3ccccc34)cc2)cc1]

The same operation as in Synthesis Example 1 was performed except that 2-aminobiphenyl was used instead of N-(4-(dibenzo[b,d]furan-4-yl)phenyl)-[1,1′-biphenyl]-4-amine used in Synthesis Example 1 to obtain a solid of 4.98 g. The yield was 60%.

As a result of mass spectrum analysis, the obtained product was Compound Inv-16, and m/e was 673 with respect to the molecular weight of 673.28.

### Synthesis Example 17: Synthesis of Compound Inv-17

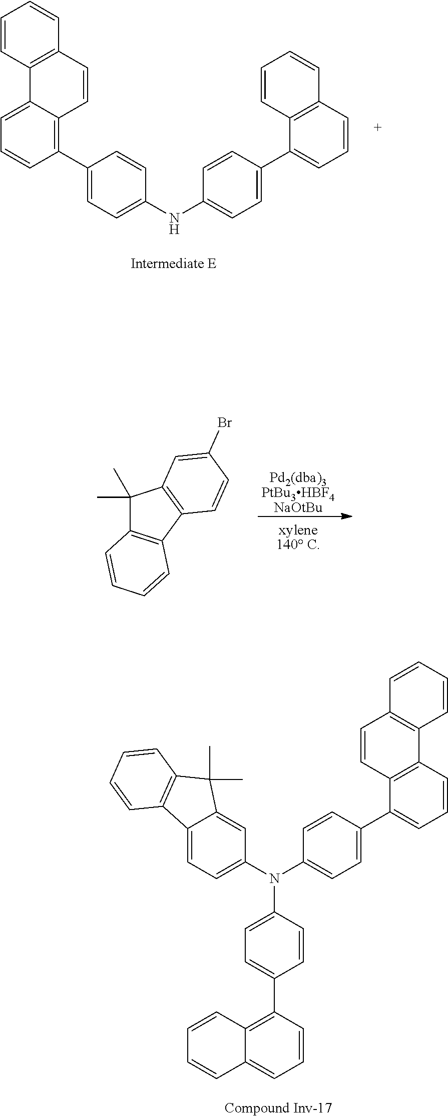 [SMILES: c1ccc2c(-c3ccc(Nc4ccc(-c5cccc6c5ccc5ccccc56)cc4)cc3)cccc2c1 ; CC1(C)c2ccccc2-c2ccc(Br)cc21 ; "Pd2(dba)3PtBu3·HBF4NaOtBu" ; "xylene140° C." ; CC1(C)c2ccccc2-c2ccc(N(c3ccc(-c4cccc5ccccc45)cc3)c3ccc(-c4cccc5c4ccc4ccccc45)cc3)cc21]

The same operation as in Synthesis Example 1 was performed except that Intermediate E was used instead of N-(4-(dibenzo[b,d]furan-4-yl)phenyl)-[1,1′-biphenyl]-4-amine used in Synthesis Example 1, and 9-(4-bromophenyl)-9H-carbazole synthesized in the same manner as in the method described in JP 2004-217557 A was used instead of Intermediate A used in Synthesis Example 1 to obtain a solid of 3.59 g. The yield was 64%.

As a result of mass spectrum analysis, the obtained product was Compound Inv-17, and m/e was 663 with respect to the molecular weight of 663.29.

### Synthesis Example 18: Synthesis of Compound Inv-18

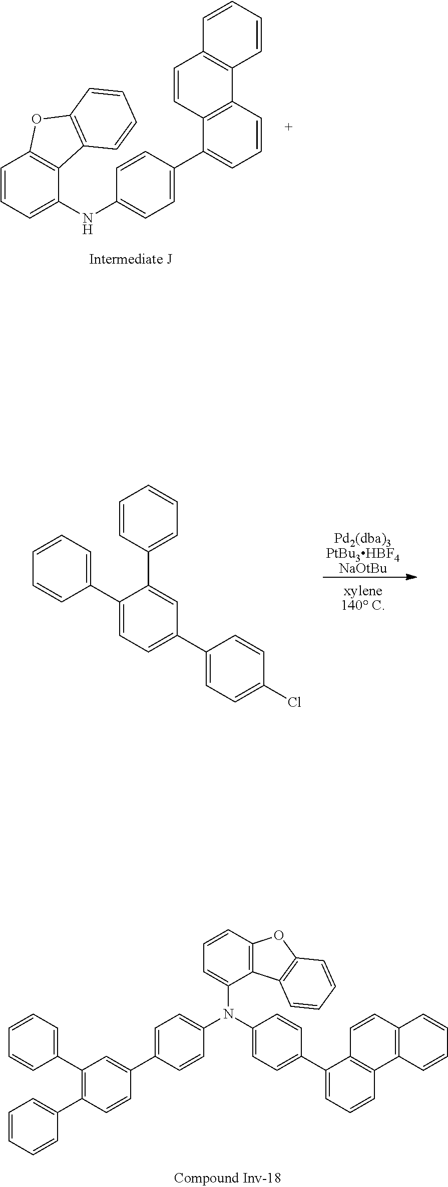 [SMILES: c1ccc2c(c1)ccc1c(-c3ccc(Nc4cccc5oc6ccccc6c45)cc3)cccc12 ; Clc1ccc(-c2ccc(-c3ccccc3)c(-c3ccccc3)c2)cc1 ; "Pd2(dba)3PtBu3·HBF4NaOtBu" ; "xylene140° C." ; c1ccc(-c2ccc(-c3ccc(N(c4ccc(-c5cccc6c5ccc5ccccc56)cc4)c4cccc5oc6ccccc6c45)cc3)cc2-c2ccccc2)cc1]

The same operation as in Synthesis Example 1 was performed except that Intermediate J was used instead of N-(4-(dibenzo[b,d]furan-4-yl)phenyl)-[1,1′-biphenyl]-4-amine used in Synthesis Example 1, and 4′-(4-chlorophenyl)-1,1′:2′,1″-terphenyl was used instead of Intermediate A used in Synthesis Example 1 to obtain a solid of 5.84 g. The yield was 70%.

As a result of mass spectrum analysis, the obtained product was Compound Inv-18, and m/e was 739 with respect to the molecular weight of 739.29.

### Synthesis Example 19: Synthesis of Compound Inv-19

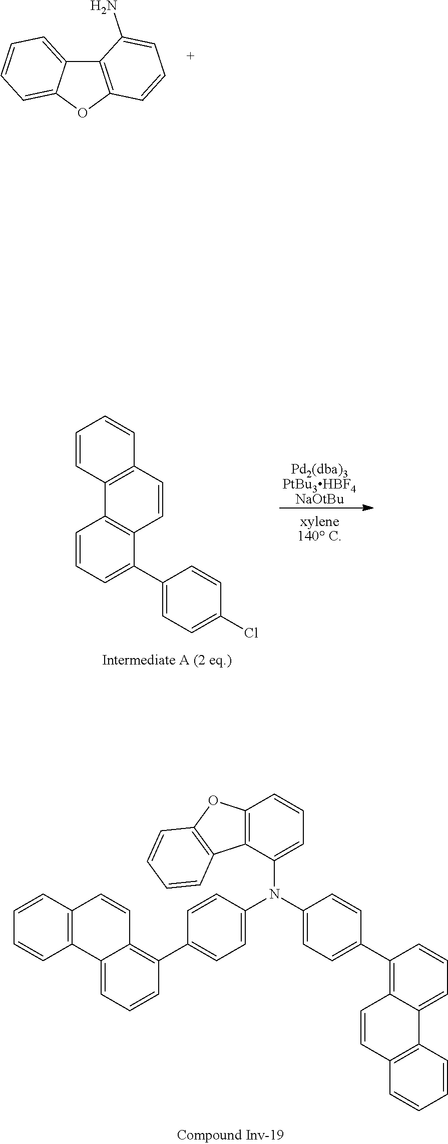 [SMILES: Nc1cccc2oc3ccccc3c12 ; Clc1ccc(-c2cccc3c2ccc2ccccc23)cc1 ; "Pd2(dba)3PtBu3·HBF4NaOtBu" ; "xylene140° C." ; c1ccc2c(c1)ccc1c(-c3ccc(N(c4ccc(-c5cccc6c5ccc5ccccc56)cc4)c4cccc5oc6ccccc6c45)cc3)cccc12]

The same operation as in Synthesis Example 1 was performed except that 1-aminodibenzofuran was used instead of N-(4-(dibenzo[b,d]furan-4-yl)phenyl)-[1,1′-biphenyl]-4-amine used in Synthesis Example 1 to obtain a solid of 3.26 g. The yield was 67%.

As a result of mass spectrum analysis, the obtained product was Compound Inv-19, and m/e was 687 with respect to the molecular weight of 687.26.

### Synthesis Example 20: Synthesis of Compound Inv-20

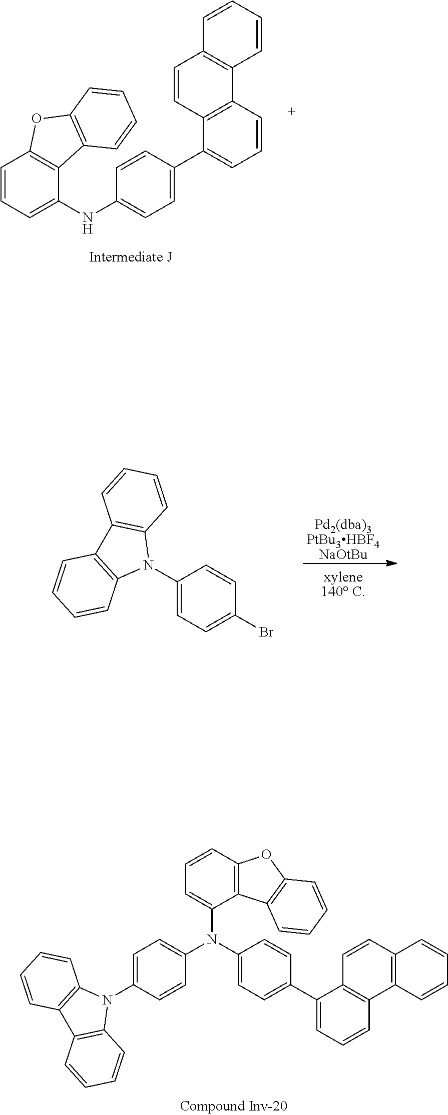 [SMILES: c1ccc2c(c1)ccc1c(-c3ccc(Nc4cccc5oc6ccccc6c45)cc3)cccc12 ; Brc1ccc(-n2c3ccccc3c3ccccc32)cc1 ; "Pd2(dba)3PtBu3·HBF4NaOtBu" ; "xylene140° C." ; c1ccc2c(c1)ccc1c(-c3ccc(N(c4ccc(-n5c6ccccc6c6ccccc65)cc4)c4cccc5oc6ccccc6c45)cc3)cccc12]

The same operation as in Synthesis Example 1 was performed except that Intermediate J was used instead of N-(4-(dibenzo[b,d]furan-4-yl)phenyl)-[1,1′-biphenyl]-4-amine used in Synthesis Example 1, and 9-(4-bromophenyl)-9H-carbazole was used instead of Intermediate A used in Synthesis Example 1 to obtain a solid of 7.99 g. The yield was 75%.

As a result of mass spectrum analysis, the obtained product was Compound 20, and m/e was 676 with respect to the molecular weight of 676.25.

### REFERENCE SIGNS LIST

1, 11, 12: Organic EL device
2: Substrate
3: Anode
4: Cathode
5: Light emitting layer
6: Hole transporting zone (hole transporting layer)
6a: Hole injecting layer
6b: First hole transporting layer
6c: Second hole transporting layer
6d: Third hole transporting layer
7: Electron transporting zone (electron transporting layer)
7a: First electron transporting layer
7b: Second electron transporting layer
10, 20, 30: Light emitting unit

1. A compound represented by the following formula (1): 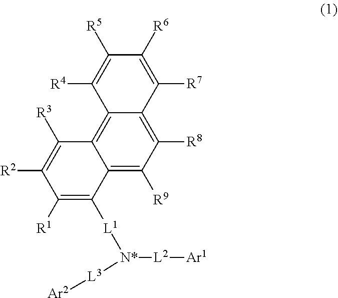
wherein
N* is a central nitrogen atom;
R¹ to R⁹ are each a hydrogen atom;
L¹ is an unsubstituted arylene group having 6 to 12 ring carbon atoms;
L² is a single bond or an unsubstituted arylene group having 6 to 12 ring carbon atoms;
L³ is a single bond or an unsubstituted arylene group having 6 to 12 ring carbon atoms;
Ar¹ is a group represented by any one of the following formulae (1-a) to (1-e); and
Ar² is a substituted or unsubstituted aryl group composed of only a 6-membered ring having 6 to 30 ring carbon atoms, or a group represented by the following formula (1-f) or (1-g): 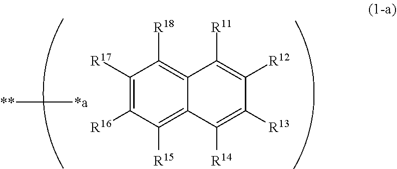
wherein
R¹¹ to R¹⁸ are each independently a hydrogen atom, a substituted or unsubstituted alkyl group having 1 to 6 carbon atoms, or a substituted or unsubstituted aryl group having 6 to 12 ring carbon atoms,
provided that,
one selected from R¹¹ to R¹⁸ is a single bond bonded to *a, and
adjacent two selected from R¹¹ to R¹⁸, which are not a single bond, are not bonded to each other, and thus do not form a ring structure; and
** represents a bonding site to L²,
provided that, when L² is a single bond, ** represents a bonding site to the central nitrogen atom N*, 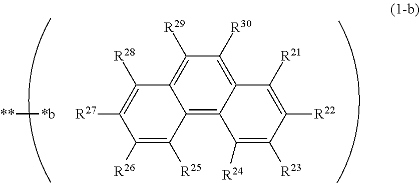
wherein
R²¹ to R³⁰ are each independently a hydrogen atom, a substituted or unsubstituted alkyl group having 1 to 6 carbon atoms, or a substituted or unsubstituted aryl group having 6 to 12 ring carbon atoms,
provided that,
one selected from R²¹ to R³⁰ is a single bond bonded to *b, and
adjacent two selected from R²¹ to R³⁰, which are not a single bond, are not bonded to each other, and thus do not form a ring structure; and
** represents a bonding site to L²,
provided that, when L² is a single bond, ** represents a bonding site to the central nitrogen atom N*, 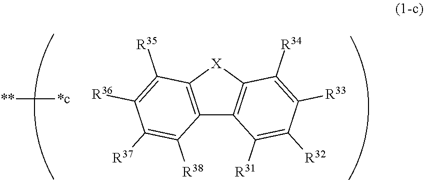
wherein
R³¹ to R³⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
X is an oxygen atom, a sulfur atom, or NR^{a};
R^{a} is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms,
provided that,
one selected from R³¹ to R³⁸ and R^{a} is a single bond bonded to *c, and
adjacent two selected from R³¹ to R³⁸ which are not a single bond may be bonded to each other to form a substituted or unsubstituted benzene ring; and
** represents a bonding site to L²,
provided that, when L² is a single bond, ** represents a bonding site to the central nitrogen atom N*, 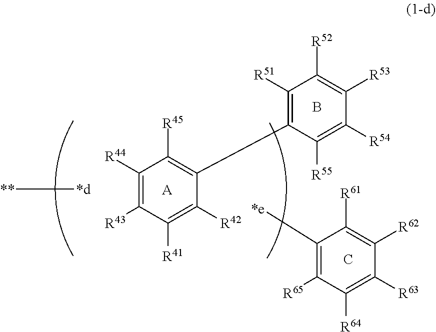
wherein
R⁴¹ to R⁴⁵ are each independently a hydrogen atom, an unsubstituted alkyl group having 1 to 6 carbon atoms, or an unsubstituted phenyl group;
R⁵¹ to R⁵⁵ and R⁶¹ to R⁶⁵ are each independently a hydrogen atom or an unsubstituted alkyl group having 1 to 6 carbon atoms,
provided that,
one selected from R⁴¹ to R⁴⁵ is a single bond bonded to *d,
another one selected from R⁴¹ to R⁴⁵ is a single bond bonded to *e,
adjacent two selected from R⁴¹ to R⁴⁵, which are not a single bond, are not bonded to each other, and thus do not form a ring structure, and
adjacent two selected from R⁵¹ to R⁵⁵ and R⁶¹ to R⁶⁵ may be bonded to each other to form a substituted or unsubstituted benzene ring; and
** represents a bonding site to L²,
provided that, when L² is a single bond, ** represents a bonding site to the central nitrogen atom N*, 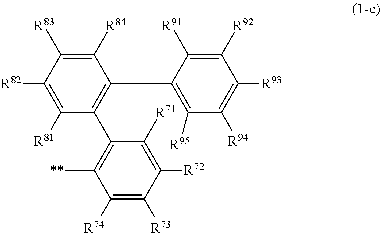
wherein
R⁷¹ to R⁷⁴, R⁸¹ to R⁸⁴, and R⁹¹ to R⁹⁵ are each independently a hydrogen atom, an unsubstituted alkyl group having 1 to 6 carbon atoms, or an unsubstituted phenyl group,
provided that,
adjacent two selected from R⁷¹ to R⁷⁴, R⁸¹ to R⁸⁴, and R⁹¹ to R⁹⁵ are not bonded to each other, and thus do not form a ring structure; and
** represents a bonding site to L²,
provided that, when L² is a single bond, ** represents a bonding site to the central nitrogen atom N*, 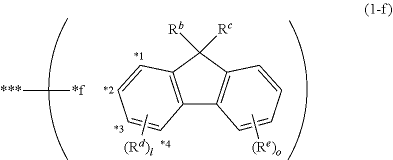
wherein
R^{b} and R^{c} are each independently an unsubstituted alkyl group having 1 to 6 carbon atoms, or an unsubstituted aryl group having 6 to 12 ring carbon atoms,
provided that,
when R^{b} and R^{c} are each an unsubstituted alkyl group having 1 to 6 carbon atoms, R^{b} and R^{c} are not bonded to each other and thus do not form a ring structure, and
when R^{b} and R^{c} are each an unsubstituted aryl group having 6 to 12 ring carbon atoms, R^{b} and R^{c} may be bonded to each other to form a substituted or unsubstituted ring;
R^{d} and R^{e} are each independently an unsubstituted aryl group having 6 to 12 ring carbon atoms;
l and o are each independently an integer of 0 to 2, in a case where 1 is 0, R^{d} is not present, and in a case where o is 0, R^{e} is not present;
*f is bonded to one carbon atom selected from carbon atoms *1 to *4 and a carbon atom contained in an unsubstituted alkyl group having 1 to 6 carbon atoms or an unsubstituted aryl group having 6 to 12 ring carbon atoms represented by R^{b} and R^{c}; and
*** represents a bonding site to L³,
provided that, when L³ is a single bond, *** represents a bonding site to the central nitrogen atom N*, 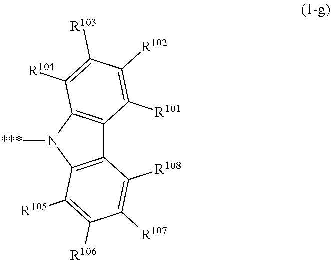
wherein
R¹⁰¹ to R¹⁰⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms,
provided that,
adjacent two selected from R¹⁰¹ to R¹⁰⁸ may be bonded to each other to form a substituted or unsubstituted benzene ring; and
*** represents a bonding site to L³,
provided that, when L³ is a single bond, *** represents a bonding site to the central nitrogen atom N*.

2. The compound according to claim 1, wherein Ar² is a group represented by any one of the following formulae (1-h) to (1-j): 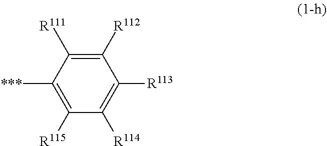
wherein
R¹¹¹ to R¹¹⁵ are each independently a hydrogen atom, a substituted or unsubstituted alkyl group having 1 to 6 carbon atoms, or a substituted or unsubstituted aryl group having 6 to 12 ring carbon atoms,
provided that,
adjacent two selected from R¹¹¹ to R¹¹⁵ are not bonded to each other, and thus do not form a ring structure; and
*** represents a bonding site to the central nitrogen atom N*, 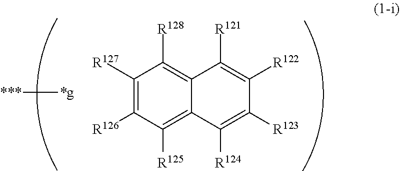
wherein
R¹²¹ to R¹²⁸ are each independently a hydrogen atom, a substituted or unsubstituted alkyl group having 1 to 6 carbon atoms, or a substituted or unsubstituted aryl group having 6 to 12 ring carbon atoms,
provided that,
one selected from R¹²¹ to R¹²⁸ is a single bond bonded to *g, and
adjacent two selected from R¹²¹ to R¹²⁸, which are not a single bond, are not bonded to each other, and thus do not form a ring structure; and
*** represents a bonding site to the central nitrogen atom N*, 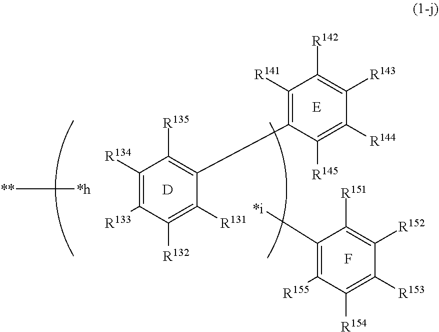
wherein
R¹³¹ to R¹³⁵ are each independently a hydrogen atom, an unsubstituted alkyl group having 1 to 6 carbon atoms, or an unsubstituted phenyl group;
R¹⁴¹ to R¹⁴⁵ and R¹⁵¹ to R¹⁵⁵ are each independently a hydrogen atom or an unsubstituted alkyl group having 1 to 6 carbon atoms,
provided that,
one selected from R¹³¹ to R¹³⁵ is a single bond bonded to *h,
another one selected from R¹³¹ to R¹³⁵ is a single bond bonded to *i,
adjacent two selected from R¹³¹ to R¹³⁵, which are not a single bond, are not bonded to each other, and thus do not form a ring structure, and
adjacent two selected from R¹⁴¹ to R¹⁴⁵ and R¹⁵¹ to R¹⁵⁵ may be bonded to each other to form a substituted or unsubstituted benzene ring; and
*** represents a bonding site to the central nitrogen atom N*.

3. The compound according to claim 1, wherein L¹ is a phenylene group or a biphenylene group.

4. The compound according to claim 1, wherein L¹ is a phenylene group.

5. The compound according to claim 1, wherein the compound is represented by the following formula (1A) or (1B): 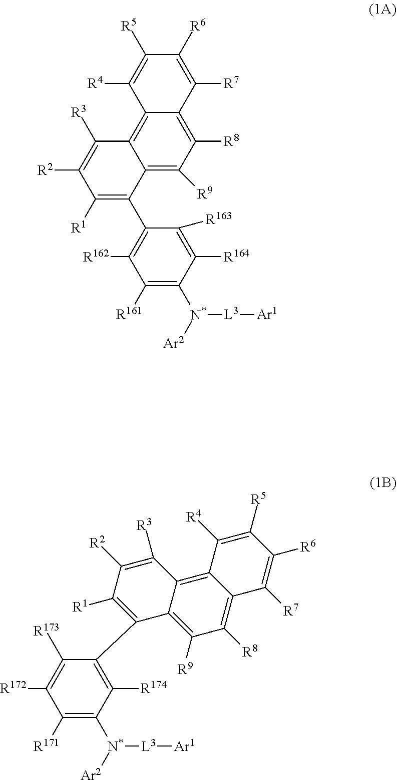
wherein
R¹⁶¹ to R¹⁶⁴ and R¹⁷¹ to R¹⁷⁴ are each a hydrogen atom; and
N*, R¹ to R⁹, L², Ar¹, and Ar² are as defined in the formula (1).

6. The compound according to claim 1, wherein L² is a phenylene group.

7. The compound according to claim 1, wherein L³ is a phenylene group.

8. The compound according to claim 1, wherein Ar¹ is a group represented by any one of the formulae (1-a) to (1-c).

9. The compound according to claim 8, wherein Ar² is a group represented by any one of the formulae (1-g) to (1-i):

10. The compound according to claim 1, wherein the compound contains at least one deuterium atom.

11. A material for an organic electroluminescent device, comprising the compound according to claim 1.

12. The material for an organic electroluminescent device according to claim 11, wherein the compound is a hole transporting layer material.

13. An organic electroluminescent device, comprising:
a cathode;
an anode; and
one or more organic layers intervening between the cathode and the anode,
wherein the one or more organic layers comprises a light emitting layer, and
wherein at least one layer of the one or more organic layers comprises the compound according to claim 1.

14. The organic electroluminescent device according to claim 13, wherein the one or more organic layers comprises a hole transporting zone between the anode and the light emitting layer, and
wherein the hole transporting zone comprises the compound.

15. The organic electroluminescent device according to claim 14, wherein the hole transporting zone comprises a first hole transporting layer on an anode side and a second hole transporting layer on a cathode side, and
wherein at least one of the first hole transporting layer and the second hole transporting layer contains the compound.

16. The organic electroluminescent device according to claim 15, wherein the second hole transporting layer contains the compound.

17. (canceled)

18. The organic electroluminescent device according to claim 15, wherein a total of a thickness of the first hole transporting layer and a thickness of the second hole transporting layer is 30 nm or more and 150 nm or less.

19. The organic electroluminescent device according to claim 13, wherein the light emitting layer is a single layer.

20. (canceled)

21. The organic electroluminescent device according to claim 13, wherein the light emitting layer comprises a fluorescent dopant material.

22. An electronic device comprising the organic electroluminescent device according to claim 13.
